(19) United States
(12) Patent Application Publication
(10) Pub. No.: US 2026/0132143 A1
(43) Pub. Date: May 14, 2026

(54) HETEROCYCLIC INHIBITORS OF KRAS G12C MUTANT PROTEINS AND USES THEREOF

(71) Applicant: AMGEN INC., THOUSAND OAKS, CA (US)

(72) Inventors: BRIAN A. LANMAN, WOODLAND HILLS, CA (US); SHON BOOKER, SHERMAN OAKS, CA (US); JOHN R. BUTLER, THOUSAND OAKS, CA (US); EMIL GLIBSTRUP, COPENHAGEN (DK); DAVID HUANG, THOUSAND OAKS, CA (US); BIRGITTE W. HUSEMOEN, COPENHAGEN (DK); MATTHEW R. KALLER, VENTURA, CA (US); TODD J. KOHN, THOUSAND OAKS, CA (US); SEBASTIAN LETH-PETERSEN, COPENHAGEN (DK); PATRICIA LOPEZ, WOODLAND HILLS, CA (US); QINGYIAN LIU, CAMARILLO, CA (US); VU VAN MA, OAK PARK, CA (US); FRANCESCO MANONI, NEWBURY PARK, CA (US); JORGE PEIRO CADAHIA, MADRID (ES); ALEXANDER J. PICKRELL, WESTLAKE VILLAGE, CA (US); SLAVKO RAST, VALBY (DK); NURIA A. TAMAYO, NEWBURY PARK, CA (US); HUI-LING WANG, THOUSAND OAKS, CA (US); WENHAN ZHANG, THOUSAND OAKS, CA (US); KAI ZHU, WESTLAKE VILLAGE, CA (US); ABHISEK BANERJEE, Karnataka (IN)

(73) Assignee: AMGEN INC., THOUSAND OAKS, CA (US)

(21) Appl. No.: 19/118,045

(22) PCT Filed: Oct. 5, 2023
(86) PCT No.: PCT/US2023/034537

§ 371 Date: Apr. 3, 2025

Related U.S. Application Data
(60) Provisional application No. 63/413,397, filed on Oct. 5, 2022.

(51) Int. Cl. C07D 491/107; A61K 31/506; A61K 31/5377; A61K 31/5383; A61K 31/55; A61K 45/06; A61P 35/00; C07B 59/00; C07D 401/14; C07D 403/14; C07D 405/14; C07D 413/14; C07D 417/14; C07D 451/02; C07D 451/14; C07D 471/04; C07D 471/08; C07D 487/04; C07D 487/10; C07D 491/048; C07D 498/04

(52) U.S. Cl. CPC C07D 491/107; A61K 31/506; A61K 31/5377; A61K 31/5383; A61K 31/55; A61K 45/06; A61P 35/00; C07B 59/002; C07D 401/14; C07D 403/14; C07D 405/14; C07D 413/14; C07D 417/14; C07D 451/02; C07D 451/14; C07D 471/04; C07D 471/08; C07D 487/04; C07D 487/10; C07D 491/048; C07D 498/04

(57) ABSTRACT

The present disclosure provides compounds having activity as inhibitors of the G12C mutant KRAS protein, pharmaceutical compositions comprising the compounds, and methods of treating certain disorders, such as cancer, including but not limited to lung, pancreatic, and colorectal cancer. In particular, the disclosure provides compounds of Formula (I), and pharmaceutically acceptable salts thereof, wherein the substituents arm as described.

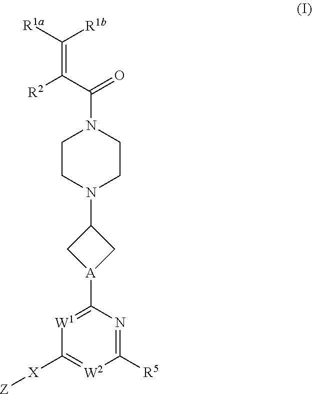

### CROSS-REFERENCE TO RELATED APPLICATIONS

This application claims the benefit of priority to U.S. Provisional Patent Application No. 63/413,397, filed Oct. 5, 2022, which is hereby incorporated by reference in its entirety, and for all purposes as if fully set forth herein.

### FIELD

The present disclosure relates generally to compounds having activity as inhibitors of the G12C-mutant KRAS protein, pharmaceutical compositions comprising the compounds, and uses and methods of treating disorders such as cancer, including but not limited to lung, pancreatic and colorectal cancer.

### BACKGROUND

The KRAS oncoprotein is a G-protein that couples extracellular mitogenic signaling to intracellular, pro-proliferative responses. KRAS functions as a molecular “on/off” switch, alternating between an inactive GDP-bound state and an active GTP-bound state. Transition between these states is facilitated by guanine nucleotide-exchange factors. Mitogen stimulation can induce GTP binding, which results in a conformational change that enables KRAS to interact with downstream effector proteins, leading to cellular proliferation. In normal cells, the pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation. See Simanshu et al., Cell 2017, 170, 17-33.

Attempts to develop inhibitors of mutated KRAS proteins have historically been thwarted by the picomolar affinity with which KRAS binds to GDP and GTP, as well as the absence of druggable pockets on the surface of the protein. Sec Cox et al., Nat. Rev. Drug Discov. 2014, 13, 828-851, Covalent inhibitors of the G12C mutant of KRAS (“KRAS^{G12C}”) have been identified. These inhibitors can bind to a previously unrecognized allosteric pocket on GDP-KRAS^{G12C}, preventing its subsequent activation. See O'Bryan, J. P. Pharmacol. Res. 2019, 139, 503-511 and Ostrem et al., Nature 2013, 503, 548-551. This discovery brought about significant new efforts in KRAS inhibitor research, recently culminating in the entry of KRAS inhibitors into human clinical trials. While some progress has been made, the need for further KRAS^{G12C} inhibitors for the treatment of disorders, such as cancer, remains.

### SUMMARY

In one aspect, the disclosure provides a compound of Formula (I):

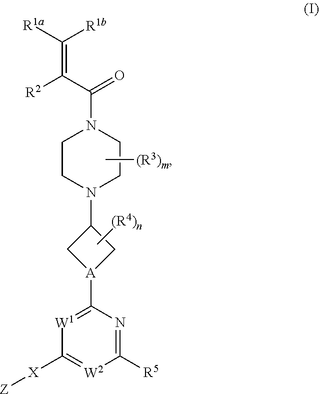

or a pharmaceutically acceptable salt thereof,
wherein:
m is 0, 1, 2, 3, or 4;
n is 0, 1, or 2;
A is N, CH, C-halo, C—CN, C—C₁₋₃ alkyl, C—C₁₋₃ haloalkyl, C—C₁₋₃ alkyleneOH, or C—C₀₋₃ alkylene-C₁₋₃ alkoxy;
each of W¹ and W² independently is N, CH, C-halo, C—CN, C—C₁₋₃ alkyl, C—C₂₋₃ alkenyl, C—C₂₋₃ alkynyl, C—C₁₋₃ haloalkyl, C—C₁₋₃ alkyleneOH, or C—C₀₋₃ alkylene-C₁₋₄ alkoxy, wherein each of the alkenyl and alkynyl is optionally substituted with 1-3 substituents and each substituent independently is halo, C₁₋₃ haloalkyl, C₀₋₃ alkyleneOH, or C₀₋₃ alkyleneC₁₋₄ alkoxy;
X is heterocycloalkyl or heterocycloalkenyl, each having 4-7 total ring atoms and 1-3 heteroatoms selected from N, O, and S, wherein each of the heterocycloalkyl and heterocycloalkenyl is optionally substituted with 1-3 substituents, and each substituent independently is halo, C₁₋₃ alkyl, C₁₋₃ haloalkyl, C₀₋₂ alkyleneOH, C₀₋₃ alkyleneC₁₋₃ alkoxy, or C₀₋₂ alkyleneCN;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S;
   wherein each of the phenyl, heteroaryl, and bicyclic rings is optionally substituted with 1-4 substituents, and each substituent independently is halo, CN, C₁₋₆ alkyl, C₁₋₆ haloalkyl, C₂₋₆ alkenyl, C₂₋₆ haloalkenyl, C₀₋₆ alkylene-OH, C₀₋₆ alkylene-C₁₋₃ alkoxy, C₀₋₆ alkylene-N(R^{N1})₂, C₀₋₂ alkylene-cycloalkyl having 3-6 total ring atoms, C₀₋₂ alkylene-heterocycloalkyl having 3-6 total ring atoms and 1-3 heteroatoms selected from N, O, and S, or C₀₋₂ alkylene-phenyl,
   wherein each of the C₁₋₆ alkyl, C₂₋₆ alkenyl, C₀₋₆ alkylene-C₁₋₃ alkoxy, cycloalkyl, heterocycloalkyl, and phenyl substituents is optionally substituted with 1-3 further substituents, and each further substituent independently is D, halo, C₁₋₃ alkyl, C₁₋₃ haloalkyl, C₁₋₂ alkyleneOH, C₁₋₂ alkylene-C₁₋₃ alkoxy, C₁₋₃ deuterated alkoxy, N(R^{N1})₂, (C═O)C₁₋₃ alkyl, cycloalkyl having 3-5 total ring atoms, heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S; or two adjacent further substituents, together with the atoms to which they are attached, form fused-cycloalkyl having 3-5 total ring atoms or fused-heterocycloalkyl having 3-5 total ring atoms and 1 or 2 heteroatoms selected from N, O, and S;
   wherein each of the foregoing cycloalkyl and heterocycloalkyl further substituents is optionally substituted with 1 or 2 substituents, and each substituent independently is halo or C₁₋₃ alkyl.
each of R^{1a}, R^{1b}, and R² independently is H, D, halo, C₁₋₄ alkyl, C₁₋₄ haloalkyl, C₁₋₂ alkylene-OH, C₀₋₂ alkylene-C₁₋₄ alkoxy, C₀₋₂ alkylene-C₁₋₄ haloalkoxy, C₀₋₂ alkylene-CN, C₀₋₂ alkylene-N(R^{N1})₂, C₁₋₂ alkylene-heterocycloalkyl having 3-6 total ring atoms and 1 or 2 heteroatoms selected from N, O, and S, or R^{1b} and R², together with the carbon atoms to which they are attached, form

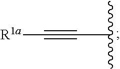

each R³ independently is C₁₋₃ alkyl, C₁₋₃ haloalkyl,

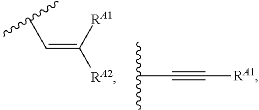

C₀₋₃ alkyleneCN, C₀₋₃ alkyleneOH, C₀₋₃ alkylene-C₁₋₃ alkoxy, oxo, spiro-cycloalkyl having 3-7 total ring atoms, spiro-cycloalkenyl having 4-7 total ring atoms, spiro-heterocycloalkyl having 3-7 total ring atoms and 1 or 2 heteroatoms selected from N, O and S, spiro-heterocycloalkenyl having 4-7 total ring atoms and 1 or 2 heteroatoms selected from N, O and S, or two adjacent R³, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1 or 2 heteroatoms selected from N, O, and S, or a fused heterocycloalkenyl ring having 4-7 total ring atoms and 1 or 2 heteroatoms selected from N, O, and S; or

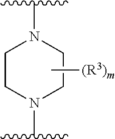

is deuterated:
each R⁴ independently is C₁₋₃ alkyl, C₁₋₃ haloalkyl, C₀₋₃ alkyleneCN, C₁₋₃ alkyleneOH, C₁₋₃ alkylene-C₁₋₃ alkoxy, oxo, spiro-cycloalkyl having 3-7 total ring atoms, or spiro-heterocycloalkyl having 4-7 total ring atoms and 1 or 2 heteroatoms selected from N, O and S;
R⁵ is C₁₋₃ haloalkyl, C₁₋₆ alkyl, C₂₋₄ alkenyl, C₂₋₄ alkynyl, halo, C₁₋₃ alkoxy, C₁₋₃ thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing independently is optionally substituted with 1-3 substituents, and each substituent independently is C₁₋₃ haloalkyl, C₁₋₃ alkylene-OH, C₀₋₆ alkylene-C₁₋₃ alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl;
each of R^{A1} and R^{A2} independently is H, C₁₋₃ alkyl, C₁₋₃ haloalkyl, or cycloalkyl having 3-5 total ring atoms; and
each R^{N1} independently is H or C₁₋₄ alkyl.

In some cases, at least one of R^{1a}, R^{1b}, and R² is H or D. In some cases, each of R^{1a}, R^{1b}, and R² is H or D. In some cases, two of R^{1a}, R^{1b}, and R² are H and one of R^{1a}, R^{1b}, and R² is halo, C₁₋₄ alkyl, C₁₋₄ haloalkyl, C₁₋₂ alkylene-OH, C₀₋₂ alkylene-C₁₋₄ alkoxy, C₀₋₂ alkylene-C₁₋₄ haloalkoxy, C₀₋₂ alkylene-CN, C₀₋₂ alkylene-N(R^{N1})₂, or C₁₋₂ alkylene-heterocycloalkyl having 3-6 total ring atoms and 1 or 2 heteroatoms selected from N, O, and S. In some cases, one of R^{1a}, R^{1b}, and R² is Br, Cl, F, CH₃, CH₂F, CHF₂, CF₃, CH₂OH, OCH₃, CH₂OCH₃, OCF₃, CH₂OCF₃, CN, CH₂CN, NH₂, N(CH₃)₂, CH₂NH₂, CH₂N(CH₃)₂, aziridin-1-yl-methyl, azetidin-1-yl-methyl, pyrrolidine-1-yl-methyl, piperidin-1-yl-methyl, or morpholin-1-yl-methyl. In some cases,

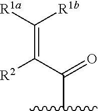

is

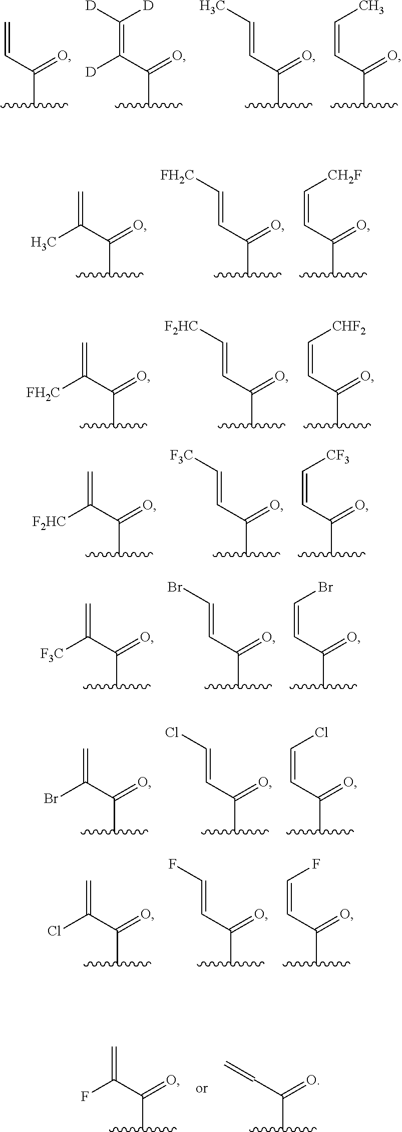

In some cases, m is 0. In some cases, in is 1. In some cases, m is 2. In some cases each R³ independently is CH₃, CH₂CH₃, CF₃, CHF₂, CH₂F,

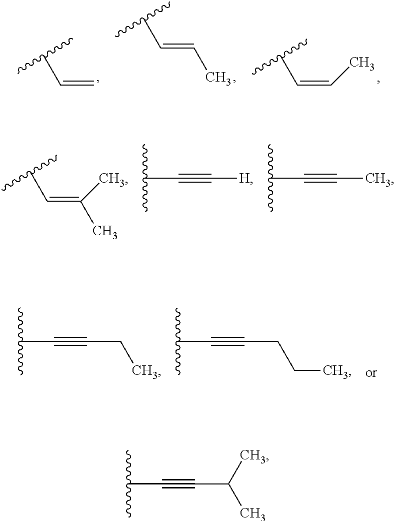

CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl, or two adjacent R³, together with the atoms to which they are attached, form a fused cyclopropyl ring or a fused cyclobutyl ring. In some cases, m is 0; or m is 1 and R³ is CH₃, CF₃, CHF₂, CH₂F, CN, CH₂CN, CH₂OH, CH₂OCH₃, or spiro-oxetanyl. In some cases,

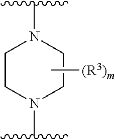

is

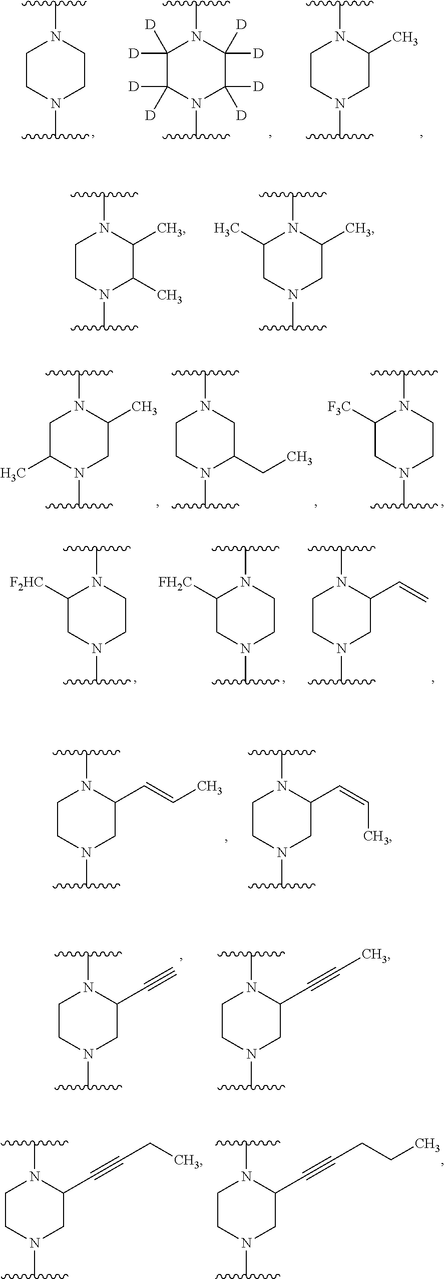

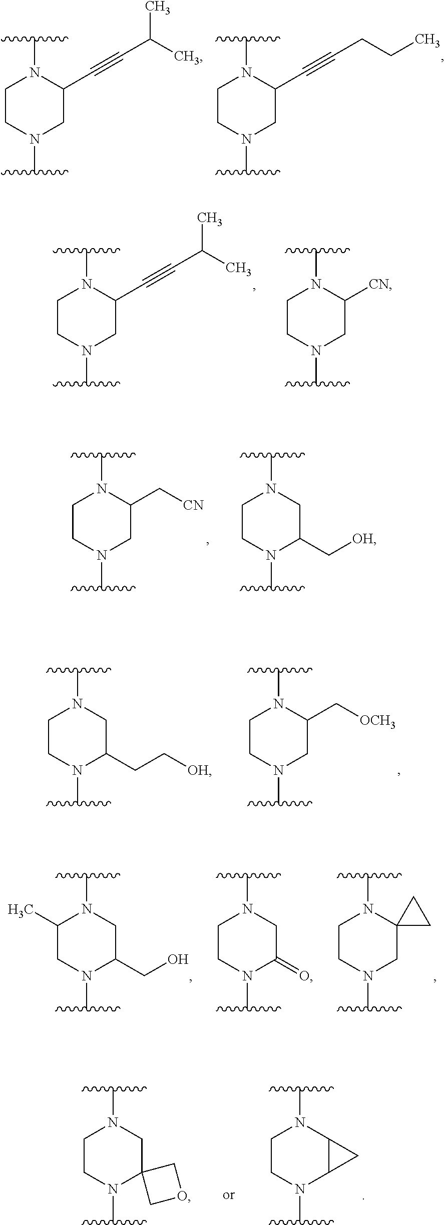

In some cases,

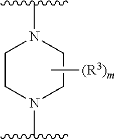

is

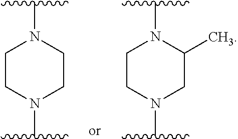 [SMILES: *N1CCN(*)CC1 ; *N1CCN(*)C(C)C1]

In some cases, A is N. In some cases, A is CH, C—F, C—Cl, C—CN, C—CH₃, C—CH₂F, C—CHF₂, C—CF₃, C—OH, C—CH₂OH, C—OCH₃, or C—CH₂OCH₃. In some cases, n is 0. In some cases, n is 1. In some cases, n is 2. In some cases, each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, CH₂OCH₃, CH₂CH₂OCH₃, oxo, spiro-cyclopropyl, spiro-cyclobutyl, or spiro-oxetanyl. In some cases,

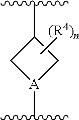

is

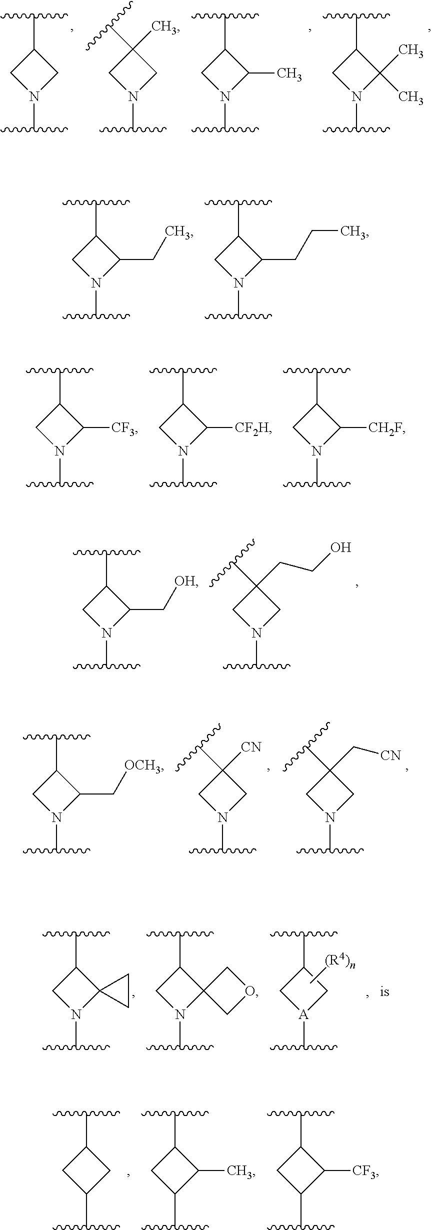

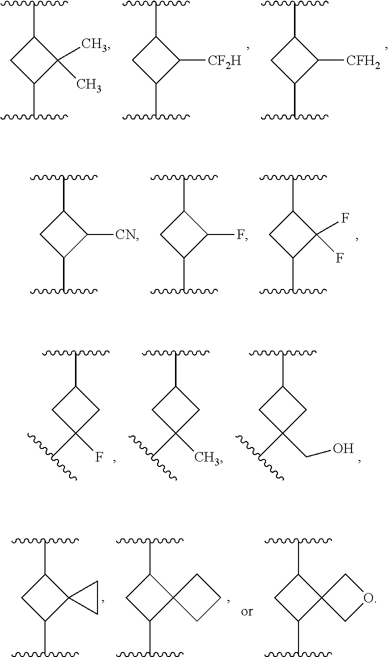

In some cases,

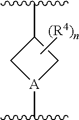

is

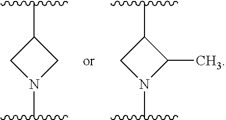 [SMILES: *C1CN(*)C1 ; *C1CN(*)C1C]

In some cases, W¹ is N. In some cases, W¹ is CH. In some cases, W¹ is C—F, C—Cl, C—CN, C—CH₃, C—CH₂CH₃, C—CH₂F, C—CHF₂, C—CF₃, C—CH═CH, C—C(OH)═CH₂, C—CH═CH(OH), C—CCH, C—OH, C—CH₂OH, C—OCH₃, or C—CH₂OCH₃. In some cases, W² is N. In some cases, W² is CH. In some cases, W² is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C—CH₂OH, C—OCH₃, or C—CH₂OCH₃. In some cases, W¹ is CH and W² is N. In some cases, R⁵ is C₁₋₃ haloalkyl. In some cases, R⁵ is CF₃, CF₂H, CFH₂. In some cases, R is CH₃, CH₂CH₃, CH₂CH₂CH₃, CH(CH₃)₂, CH═CH₂, CH═CHCH₃,

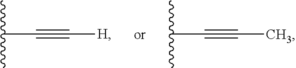

wherein each of the foregoing is optionally substituted with 1-3 substituents, and each substituent independently is C₁₋₃ haloalkyl, C₀₋₆ alkylene-OH, C₀₋₆ alkylene-C₁₋₃ alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl. In some cases, each substituent independently is CH₃, CF₃, CF₂H, CFH₂, OH, OCH₃, OCF₃, CH₂OH, CH₂OCH₃, cyclopropyl, cyclobutyl, or phenyl. In some cases, R⁵ is Br, Cl, F, OCH₃, SCH₃, CH₃, CH₂CH₃, CH₂CH₂CH₃, CH(CH₃)₂,

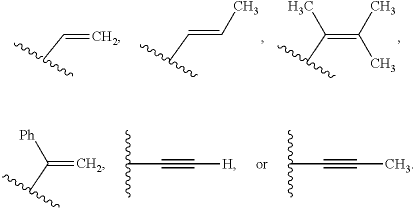

In some cases, W¹ is CH, W² is N, and R⁵ is CF₃, CF₂H, or CFH₂. In some cases.

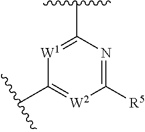

is

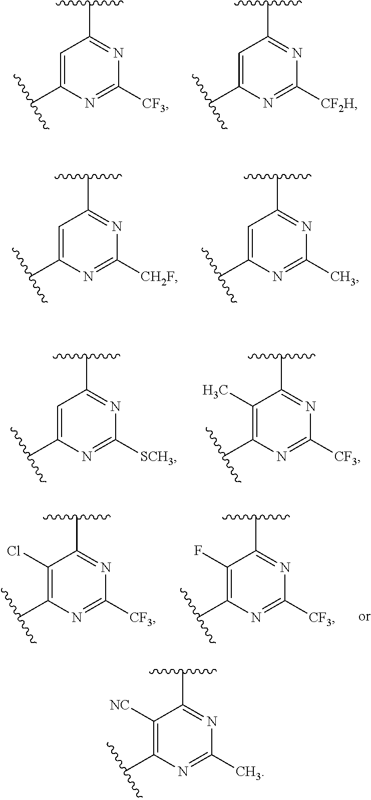

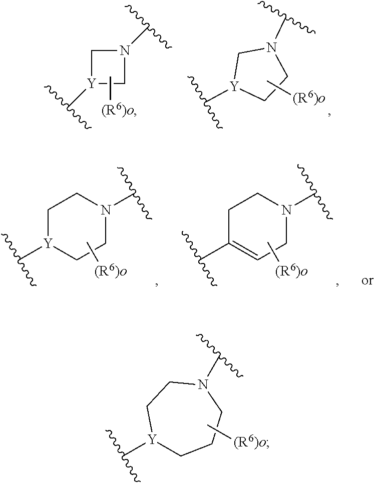

Y is N, C—H, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy; o is 0, 1, 2, 3, or 4; and each R⁶ independently is halo, CN, C₁₋₃ alkyl, C₂₋₃ alkenyl, C₁₋₃ haloalkyl, C₀₋₃ alkylene-OH, C₀₋₃ alkylene-C₁₋₃ alkoxy, deuterated C₀₋₃ alkylene-C₁₋₃ alkoxy, C₁₋₄ alkylene-N(R^{N1})₂, oxo, ═CH₂, spiro-cycloalkyl having 3-7 total atoms, spiro-cycloalkenyl having 4-7 total atoms, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 4-7 total ring atoms and 1-3 heteroatoms selected from N, O, and S, or a fused heterocycloalkenyl ring having 4-7 total ring atoms and 1 or 2 heteroatoms selected from N, O, and S; or two non-adjacent R⁶ join together to form a C₁₋₃ alkylene bridge, a C₂₋₃ alkenylene bridge, a C₁₋₃ ether bridge, or a C₁₋₃ thioether bridge; or Y and an adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1-3 heteroatoms selected from N, O, and S, or a fused heterocycloalkenyl ring having 4-7 total ring atoms and 1 or 2 heteroatoms selected from N, O, and S; wherein the cycloalkyl, cycloalkenyl, heterocycloalkyl or heterocycloalkenyl of any of the foregoing is optionally substituted with 1-4 substituents, and each substituent independently is halo, C₁₋₃ alkyl, C₁₋₃ haloalkyl, C₀₋₂ alkyleneOH, C₀₋₂ alkyleneC₁₋₃ alkoxy, or C₀₋₂ alkyleneCN; and each R^{N1} independently is H or C₁₋₄ alkyl. In some cases, X is

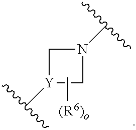

In some cases, X is

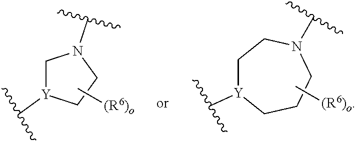

In some cases, X is

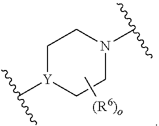

In some cases, Y is N. In some cases, Y is CH. In some cases, Y is C—F, C—Cl, C—CH₃, C—CH₂CH₃, C—CH₂F, C—CHF₂, C—CF₃, C—OH, C—CH₂OH, C—OCH₃, or C—CH₂OCH₃. In some cases, o is 0. In some cases, o is 1. In some cases, o is 2. In some cases, each R⁶ independently is Br, Cl, F, CN, CH₃, CH₂F, CHF₂, CF₃, OH, CH₂OH, OCH₃, OCD₃, CH₂OCH₃, CH₂N(CH)₂, oxo, ═CH₂, spiro-cyclopropyl, spiro-cyclobutyl, spiro-oxetanyl, or spiro-tetrahydrofuranyl, or two adjacent R⁶, together with the atoms to which they are attached, form fused-cyclopropyl, fused-cyclobutyl, or fused-cyclopentyl, and any of the foregoing spiro and fused rings is optionally substituted with 1 or 2 substituents, and each substituent independently is halo, C₁₋₃ alkyl, C₁₋₃ haloalkyl, C₀₋₂ alkyleneOH, C₀₋₂ alkyleneC₁₋₃ alkoxy, or C₀₋₂ alkyleneCN. In some cases, each substituent on the spiro and fused rings independently is F, Cl, OH, OCH₃, OCH₂CH₃, or CN. In some cases, two non-adjacent R join together to form a C₁₋₃ alkylene bridge, a C₂₋₃ alkenylene bridge, a C₁₋₃ ether bridge, or a C₁₋₃ thioether bridge. In some cases, two non-adjacent R⁶ join together to form —CH₂—, —CH₂CH₂—, —CH₂CH₂CH₂—, —CH₂—CH═CH— or —CH₂OCH₃—. In some cases, X is

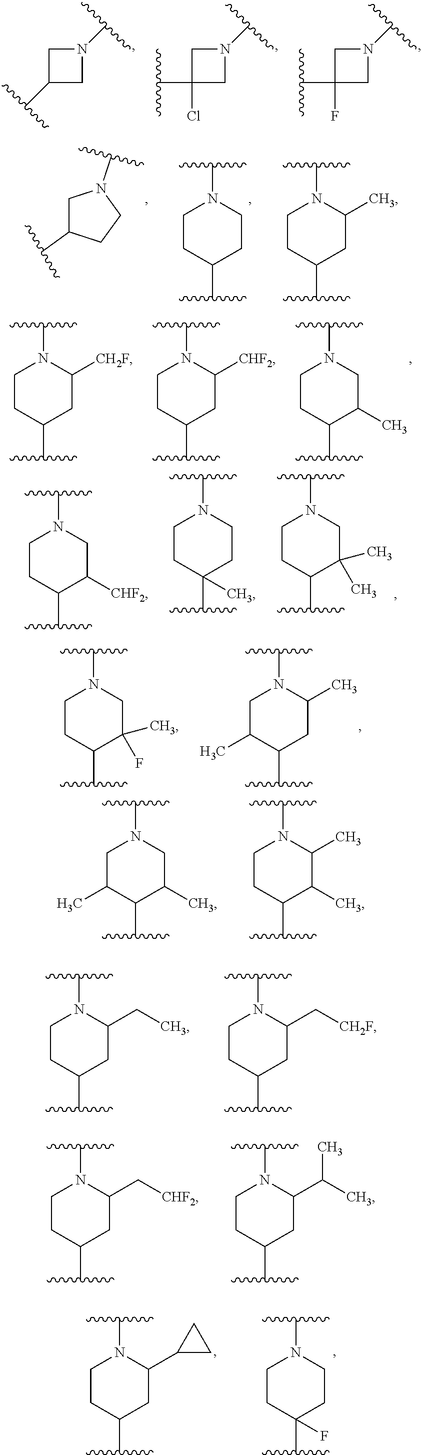

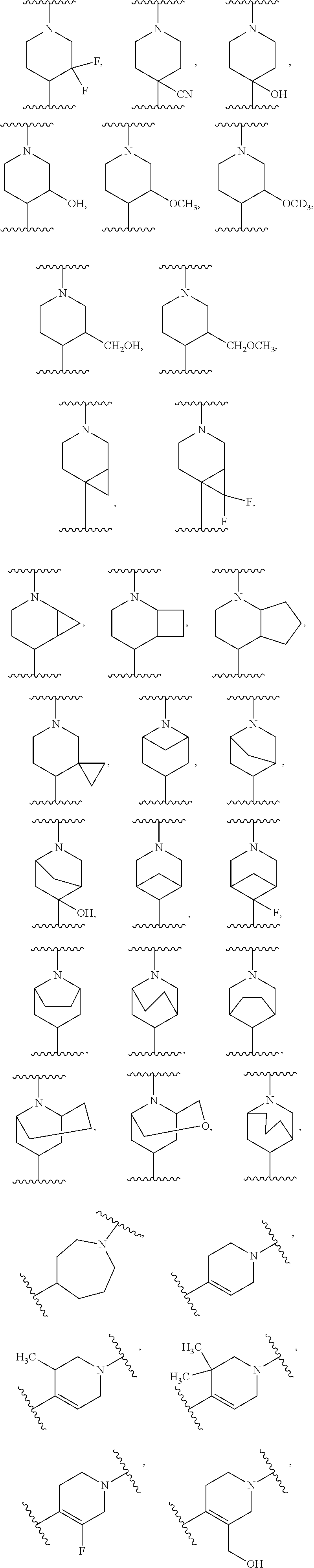

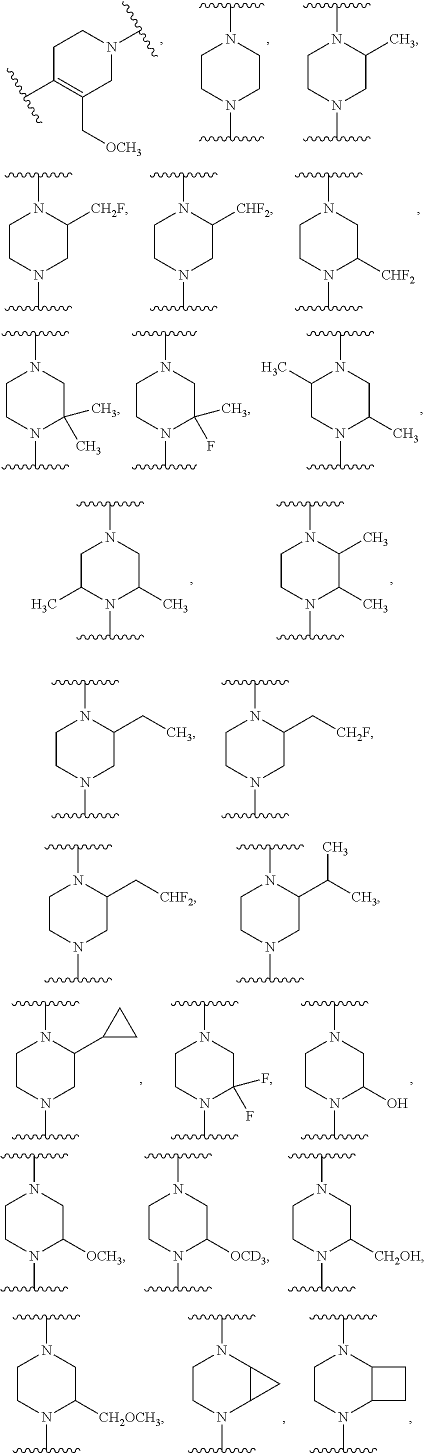

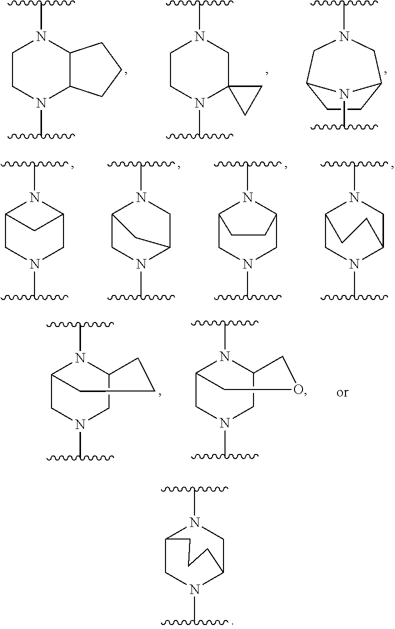

In some cases, X is

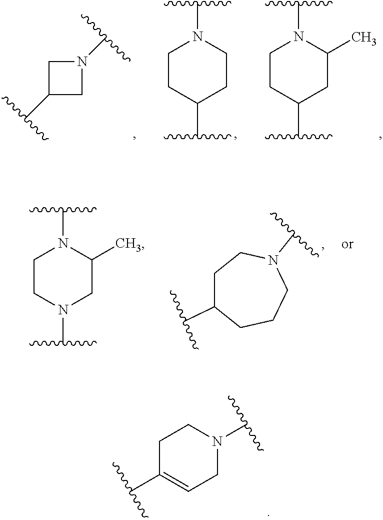

In some cases, Z is phenyl that is optionally substituted with 1-4 substituents, and each substituent independently is halo, C₀₋₃ alkyleneCN, C₀₋₃ alkyleneOH, C₀₋₃ alkylene-C₁₋₄ alkoxy, C₀₋₃ alkylene-C₁₋₄ thioalkoxy, or

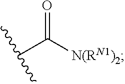

and each R^{N1} independently H or CH₃. In some cases, each substituent on the phenyl independently is F, Cl, CN, OCH₃, SCH₃, CH₂OH, or

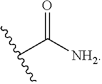 [SMILES: *C(N)=O]

In some cases, Z is

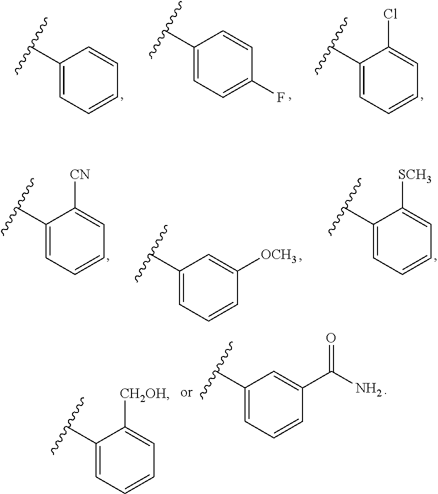

In some cases, Z is heteroaryl comprising 5 or 6 total ring atoms and 1-3 heteroatoms selected from N, O, and S, wherein the heteroaryl is optionally substituted with 1-4 substituents, and each substituent independently is halo, CN, C₁₋₆ alkyl, C₁₋₆ haloalkyl, C₂₋₆ alkenyl, C₂₋₆ haloalkenyl, C₀₋₆ alkylene-OH, C₀₋₆ alkylene-C₁₋₃ alkoxy, C₀₋₆ alkylene-N(R^{N1})₂, C₀₋₂ alkylene-cycloalkyl having 3-6 total ring atoms, C₀₋₂ alkylene-heterocycloalkyl having 3-6 total ring atoms and 1-3 heteroatoms selected from N, O, and S, or C₀₋₂ alkylene-phenyl; wherein each of the C₁₋₆ alkyl, C₂₋₆ alkenyl, C₀₋₆ alkylene-C₁₋₃ alkoxy, cycloalkyl, heterocycloalkyl, and phenyl substituents is optionally substituted with 1-3 further substituents and each further substituent independently is D, halo, C₁₋₃ alkyl, C₁₋₃ haloalkyl, C₁₋₂ alkyleneOH, C₁₋₂ alkylene-C₁₋₃ alkoxy, C₁₋₃ deuterated alkoxy. N(R^{N1})₂, (C═O)C₁₋₃ alkyl, cycloalkyl having 3-5 total ring atoms, heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S; or two adjacent further substituents, together with the atoms to which they are attached, form fused-cycloalkyl having 3-5 total ring atoms or fused-heterocycloalkyl having 3-5 total ring atoms and 1 or 2 heteroatoms selected from N, O, and S; wherein each of the foregoing cycloalkyl and heterocycloalkyl further substituents is optionally substituted with halo or C₁₋₃ alkyl; and each R^{N1} independently is H or C₁₋₃ alkyl. In some cases, Z is optionally substituted; pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl. In some cases, Z is optionally substituted: pyrazoyl or pyridyl.

In some cases, the heteroaryl is substituted with 1 or 2 substituents. In some cases, each substituent independently is Br, Cl, F, CN, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, NH₂, CH₂NH₂, CH₂NHCH₃, CH₂N(CH₃)₂, CH₂CH₂NH₂, CH₂CH₂NHCH₃, CH₂CH₂N(CH₃)₂, C₁₋₆ alkyl selected from CH₃, CH₂CH₃, CH₂CH₂CH₃, and CH(CH₃)₂, C₂₋₆ alkenyl selected from CH═CH₂, CH₂CH═CH₂, and CH—CHCH₃, C₂₋₆ alkylene-C₁₋₃ alkoxy selected from OCH₃, CH₂OCH₃, CH₂CH₂OCH₃, CH₂CH₂OCH₂CH₃, CH₂CH₂CH₂OCH₃, CH(CH₃)OCH₃, CH(CH₃)CH₂OCH₃, CH(OCH₃)CH₂OCH₃, CH(CH₃)(OCH₃)CH₂OCH₃, C(CH₃)₂OCH₃, C(CH₃)₂CH₂OCH₃, CH₂CH(CH₃)OCH₃, CH₂(CH₃)(OCH₃)OCH₃, CH₂C(CH₃)₂OCH₃, and CH₂C(CH₃)₂OCH₃, cycloalkyl selected from cyclopropyl, cyclobutyl, cyclopentyl, and cyclohexyl, or heterocycloalkyl selected from azetidinyl, pyrrolidinyl, piperidinyl, pyrazolidin 1, oxetanyl, tetrahydrofuranyl, tetrahydropyranyl, isoxazolidinyl, and morpholinyl; wherein each of the C₁₋₆ alkyl, C₂₋₆ alkenyl, C₀₋₆ alkylene-C₁₋₃ alkoxy, cycloalkyl, and heterocycloalkyl substituents independently is substituted with 1-3 further substituents and each further substituent independently is D, halo, C₁₋₃ alkyl, C₁₋₃ haloalkyl, C₁₋₂ alkyleneOH, C₁₋₂ alkylene-C₁₋₃ alkoxy, C₁₋₃ deuterated alkoxy, N(R^{N1})₂, (C═O)C₁₋₃ alkyl, cycloalkyl having 3-5 total ring atoms, heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S; or two adjacent further substituents, together with the atoms to which they are attached, form fused-cycloalkyl having 3-5 total ring atoms or fused-heterocycloalkyl having 3-5 total ring atoms and 1 or 2 heteroatoms selected from N, O, and S. In some cases, each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, azetidinyl, spiro-oxetanyl or spiro-azetidinyl; wherein each of the foregoing oxetanyl, azetidinyl, spiro-oxetanyl, and spiro-azetidinyl is optionally substituted with F, CH₃, or a combination thereof. In some cases, each further substituent independently is D, Br, Cl, F, OH, CH₃, CF₃, CF₂H, CFH₂, OCH₃, CH₂OCH₃, OCD₃, N(CH₃)₂, (C═O)CH₃,

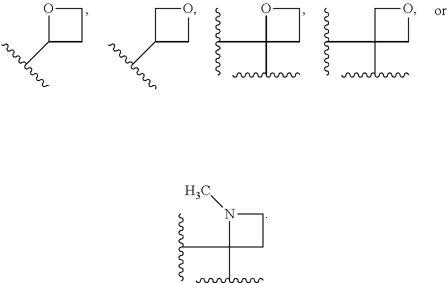

In some cases, each substituent of the heteroaryl of Z independently is Cl, F, CN, CH₃, CD₃, CH₂CH₃, CH(CH₃)₂, CF₃, CHF₂, CH₂F, CH₂CHF₂, CH, CH₂F, CH(CH₂F)₂, CH(CH₃)CH₂F, CH(CH)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH, CH₂OCH₂CH₂CH₂CH₂CH₂OCH₃, CH₂CH₂CH₂OCD₃, CH(CH₃)OCH₃, CH(CH₃)CH₂OCH₃, CH(OCH₃)CH₂OCH₃, CH(CH₃)(OCH₃)CH₂OCH₃, CH(CH₂F)(CH₃)CH₂OCD₃, CH(CH₃)CH₂OCD₃, C(CH₃)₂OCH₃, C(CH₃)₂CH₂OCH₃, C(CH₃)₂CH₂OCD₃, CH₂CH(CH₃)OCH₃, CH₂(CH₃)(OCH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

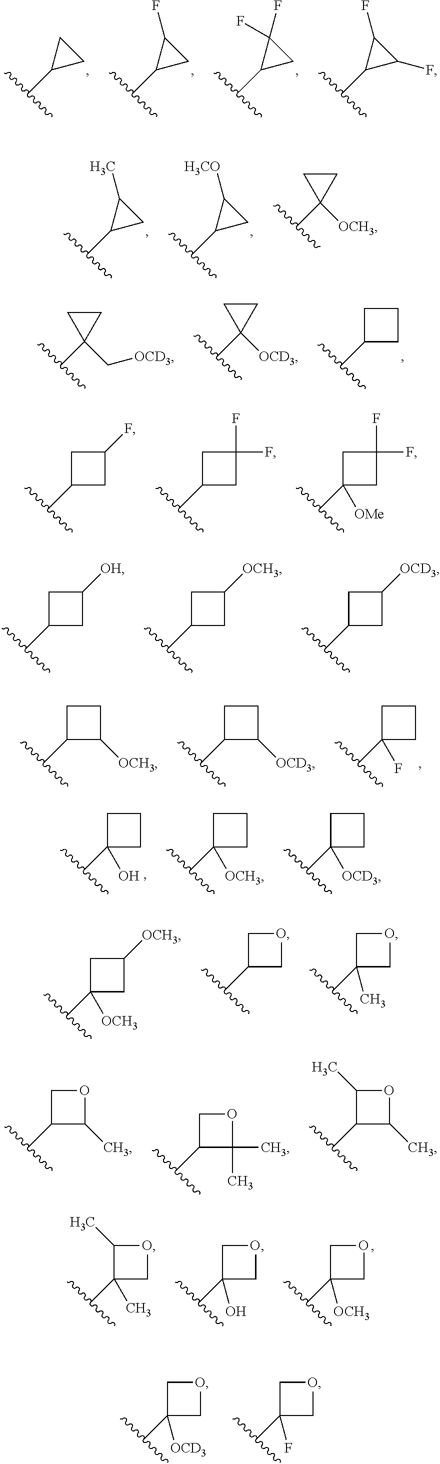

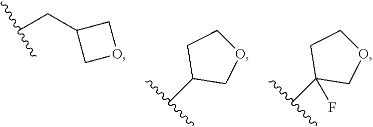 [SMILES: *CC1COC1 ; *C1CCOC1 ; *C1(F)CCOC1]

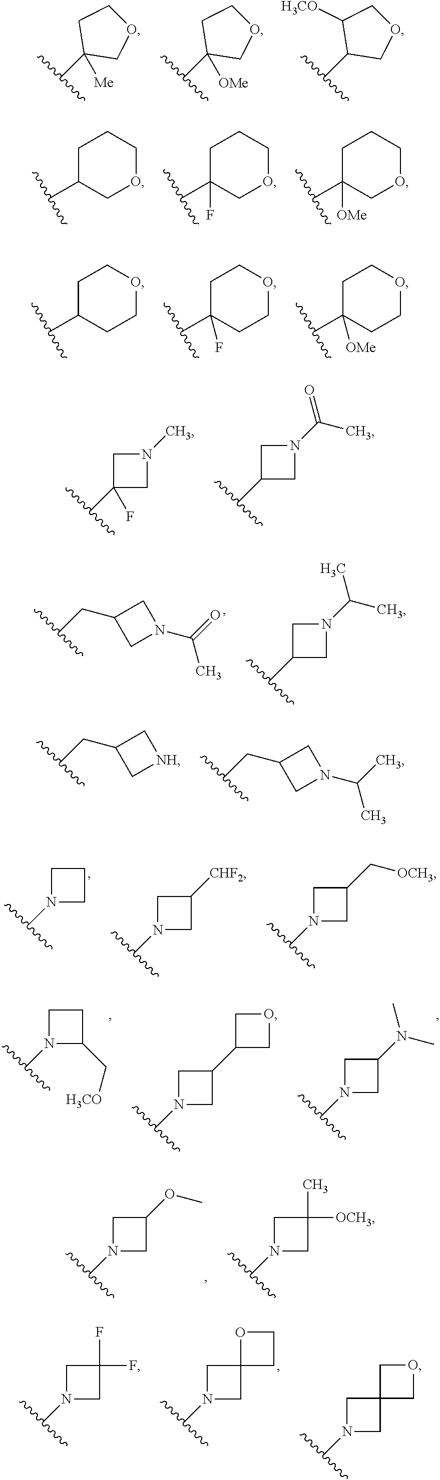

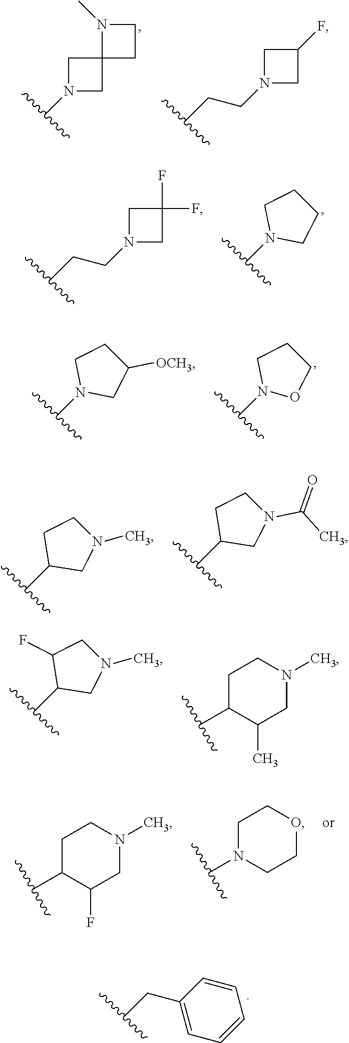

In some cases, each substituent of the heteroaryl of Z independently is CH₃, CH(CH₃)₂, C(CH₃)₂OH, CH₂OCD₃, C(CH₃)₂CH₂OH, CH₂CH₂OCH₃, CF₂CH₂OCH₃, CH₂CH₂OCD₃, CH(CH₃)OCH₃, CH₂CH₂CH₂OCH₃, CH₂CH(CH₃)OCH₃,

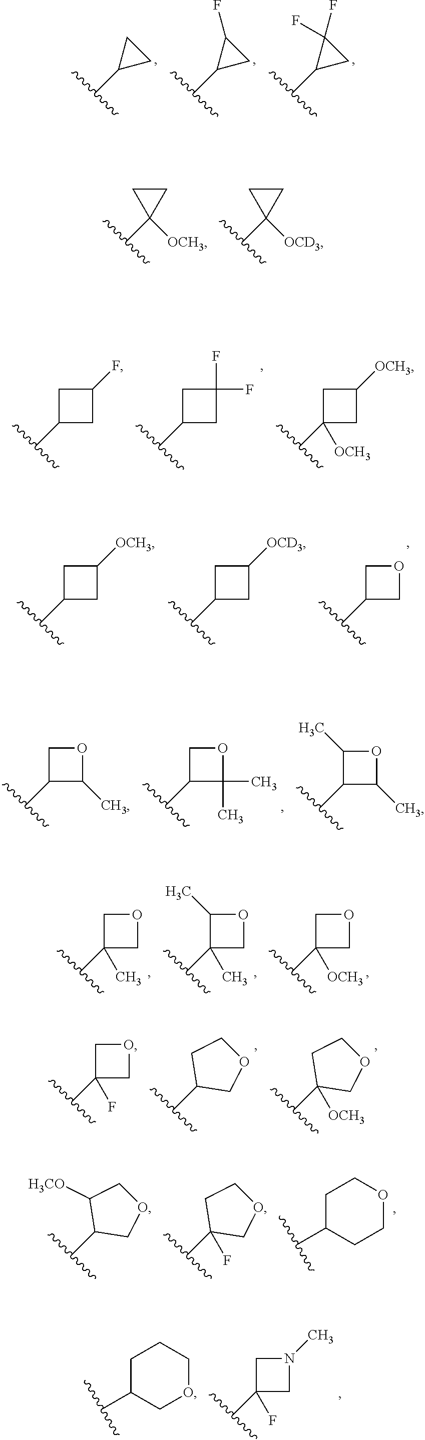

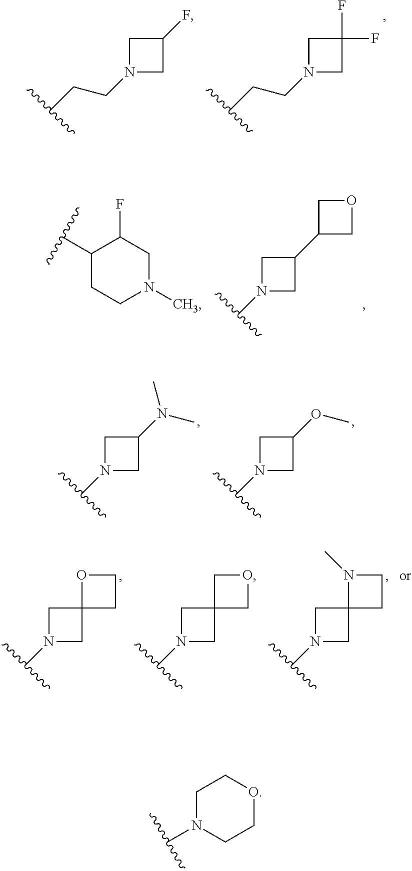

In some cases, Z is

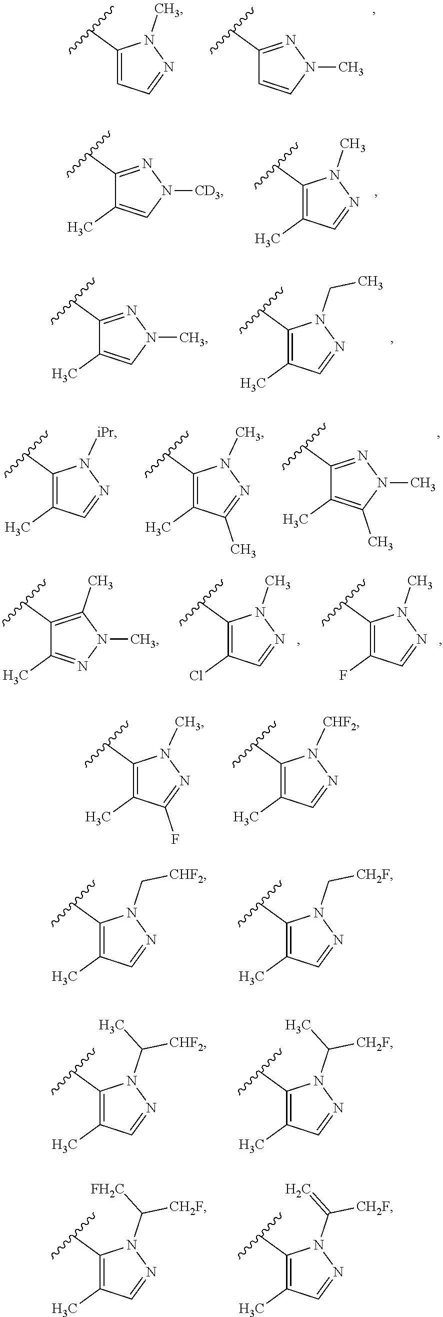

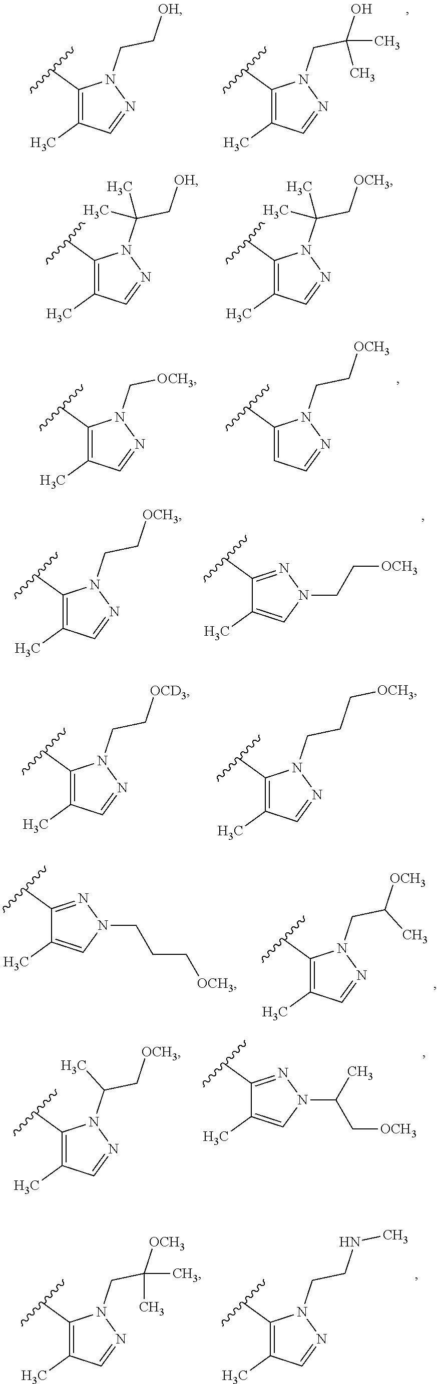

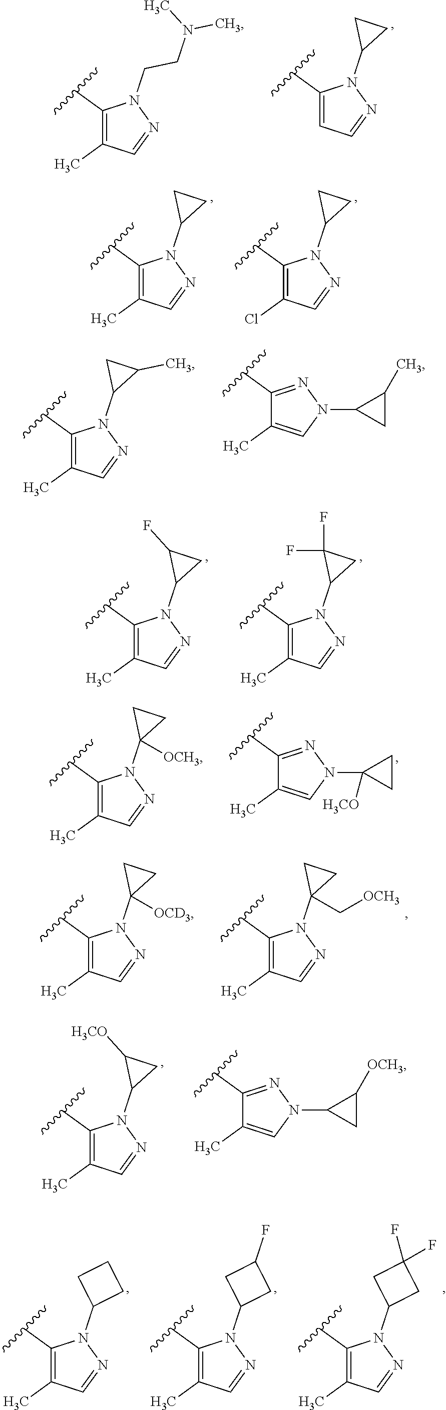

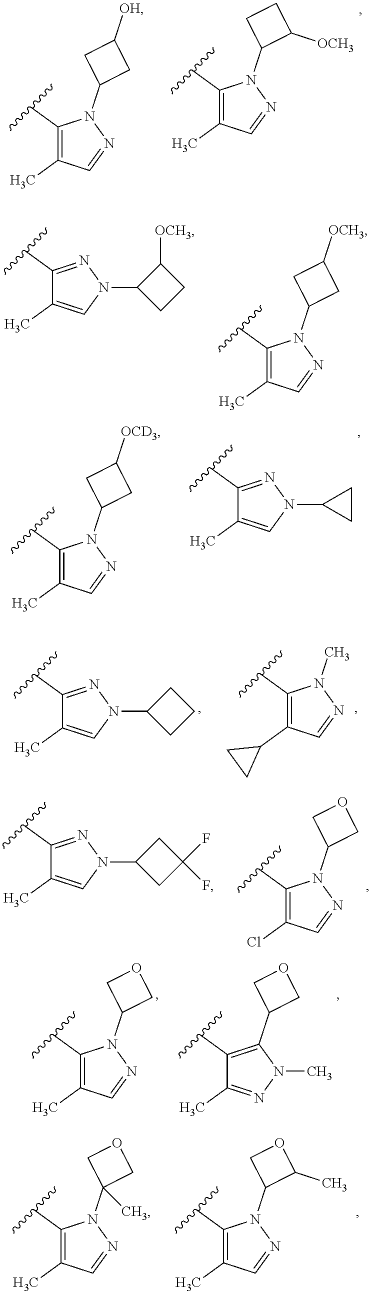

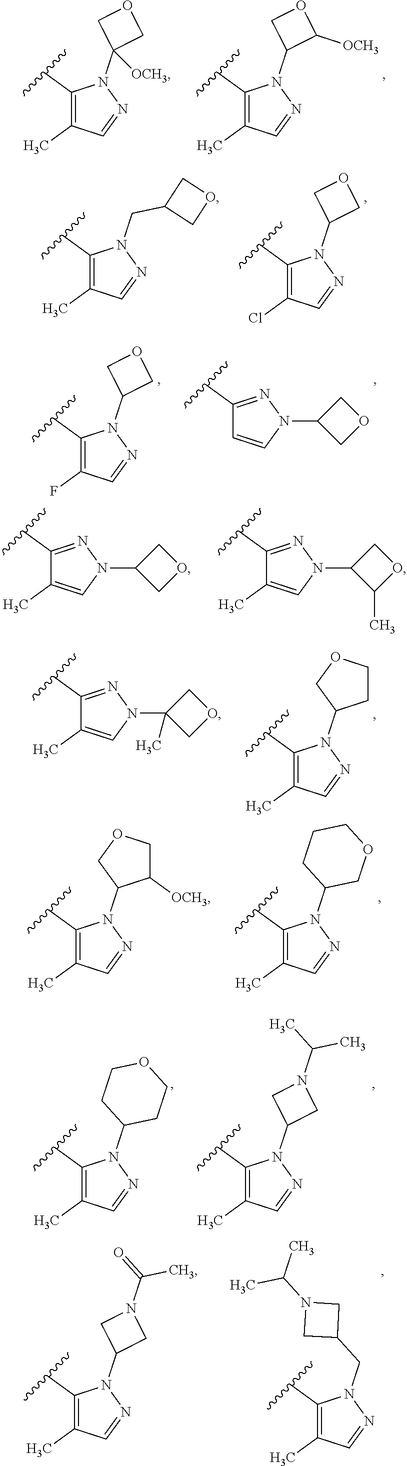

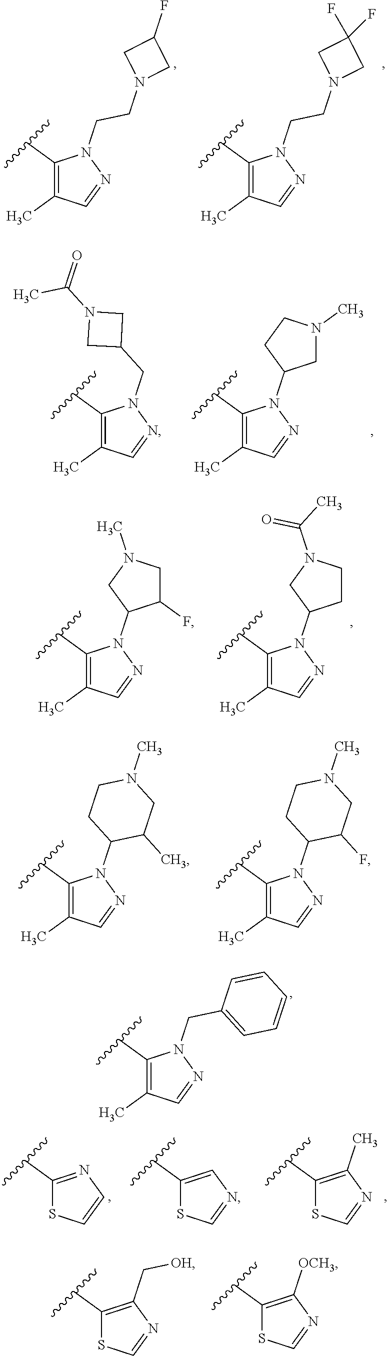

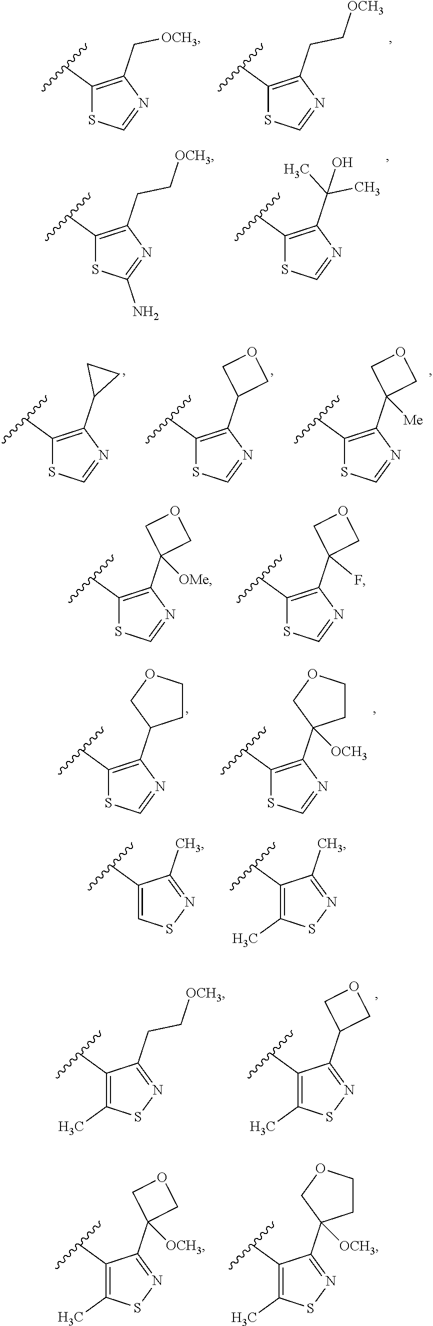

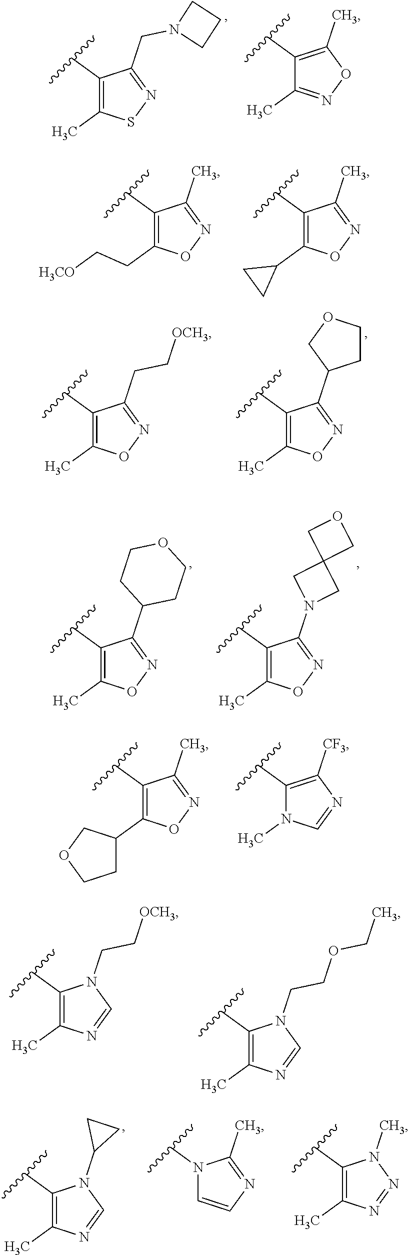

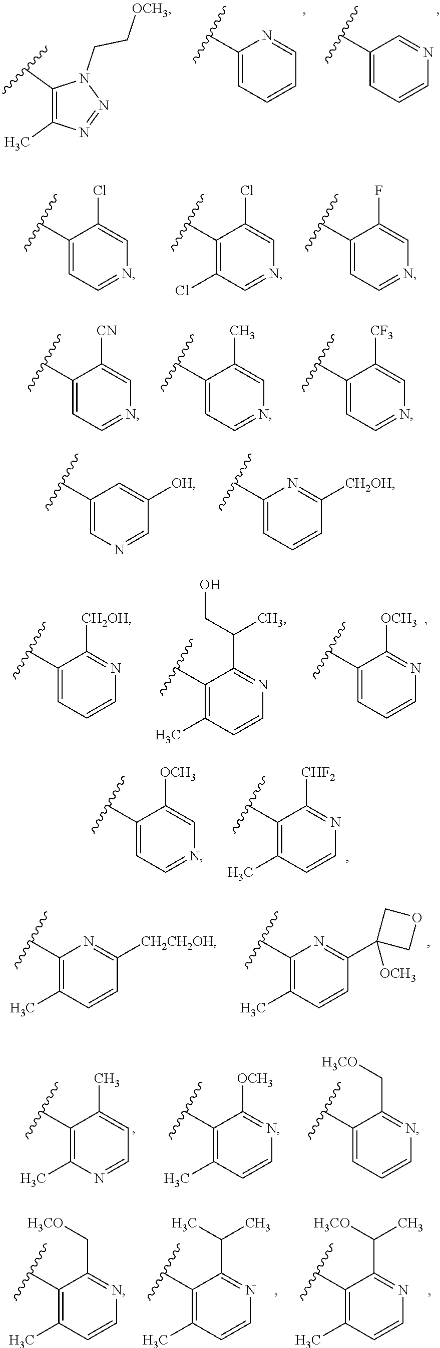

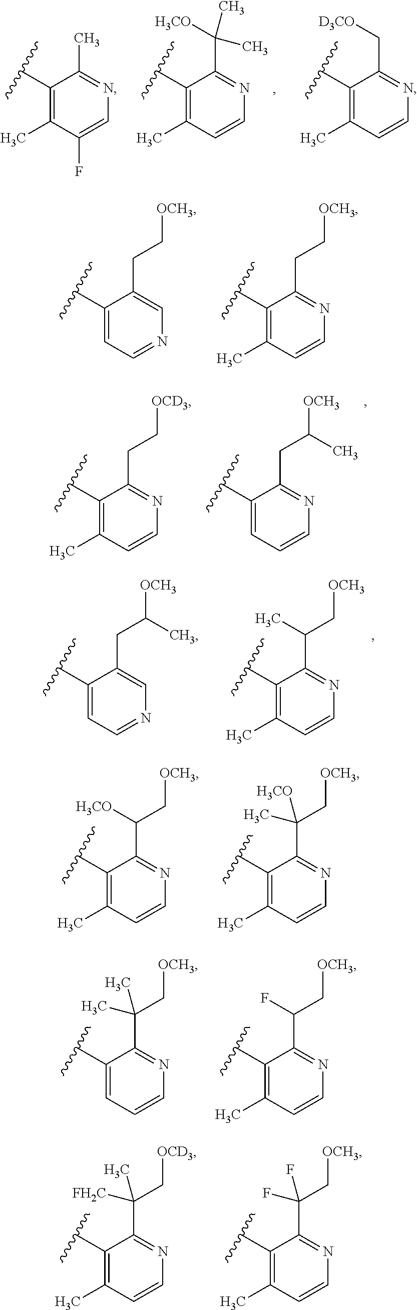

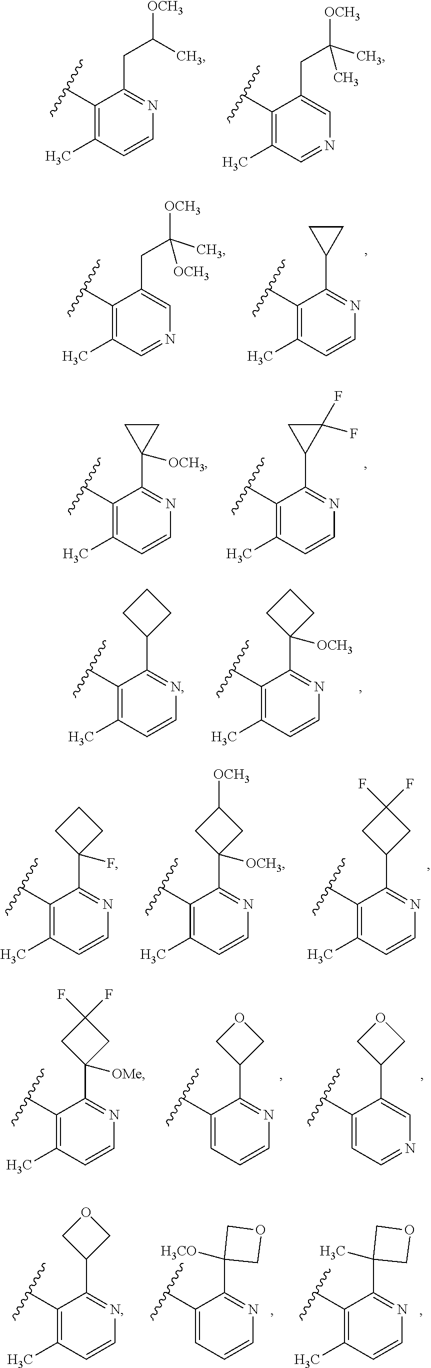

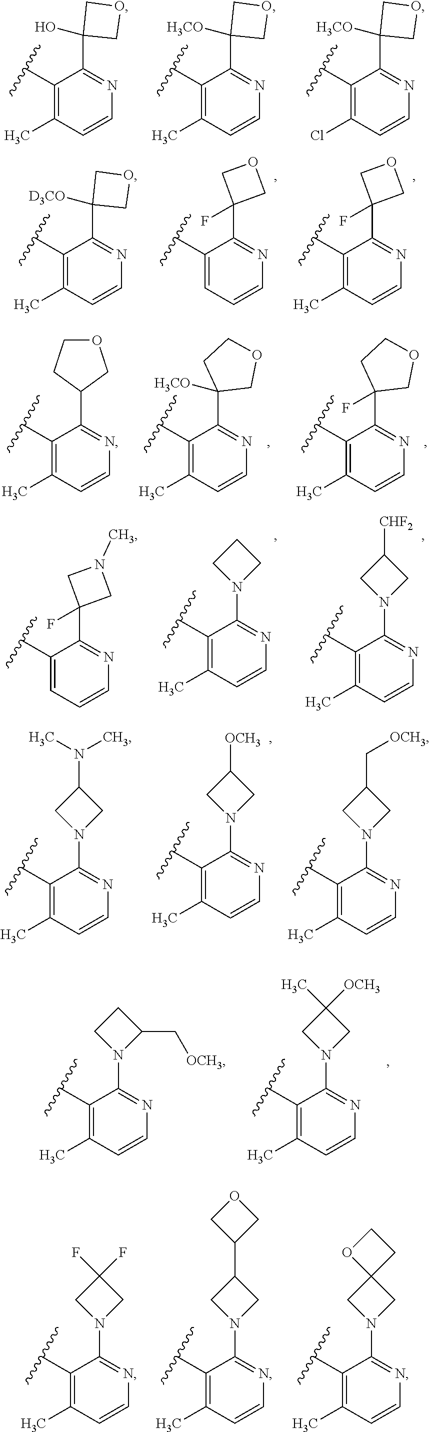

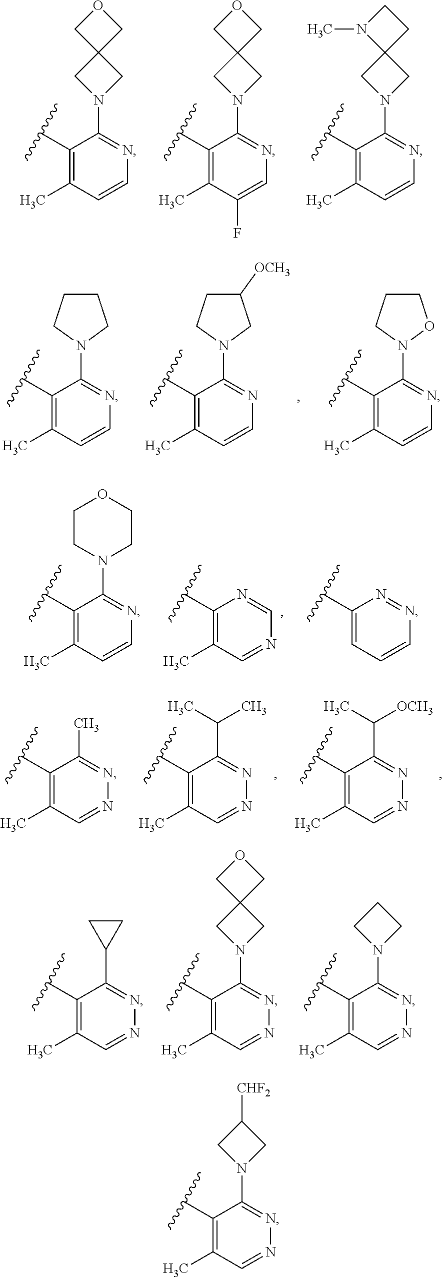

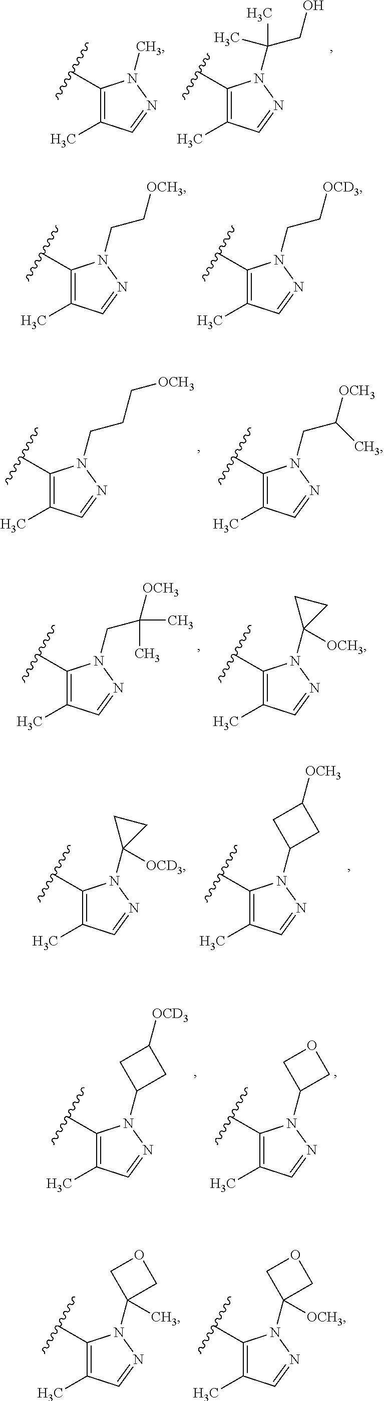

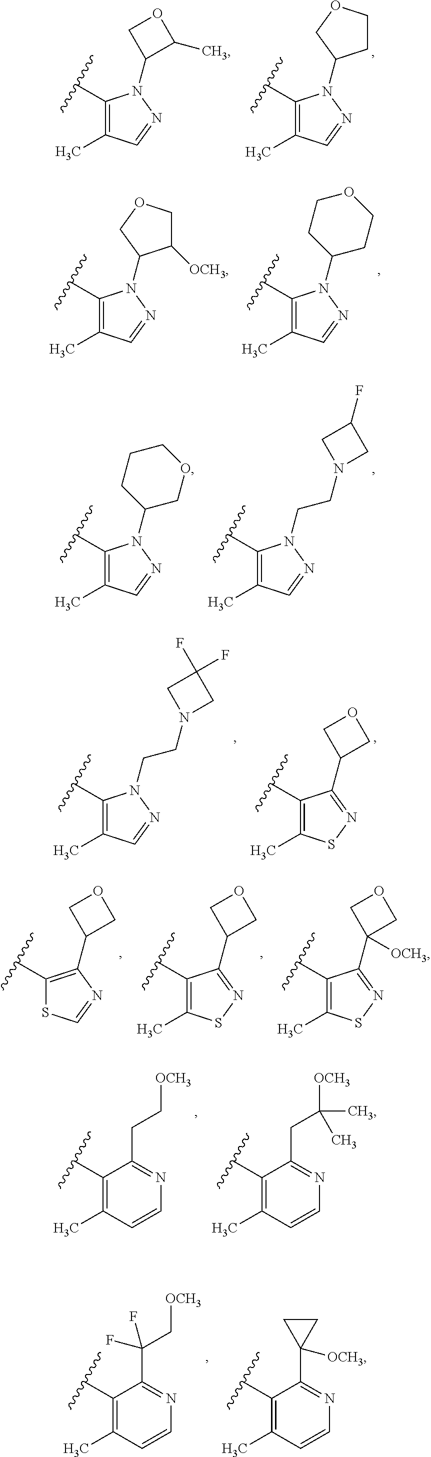

In some cases, Z is

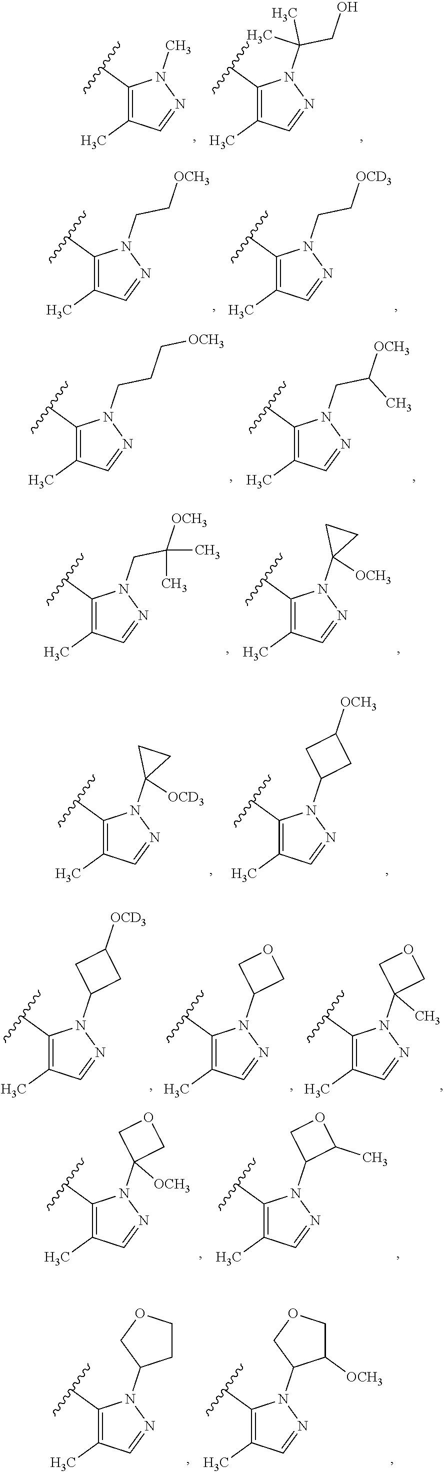

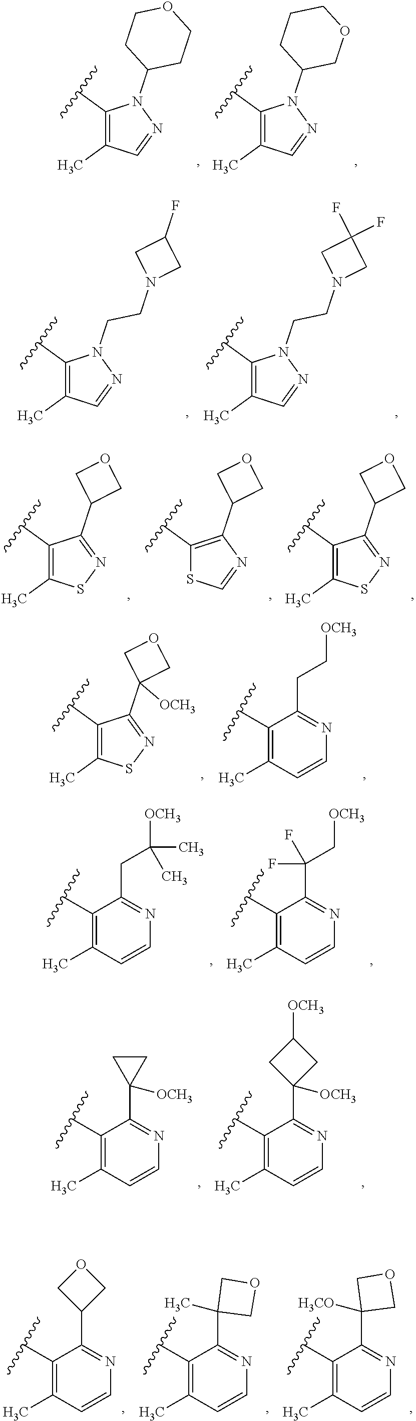

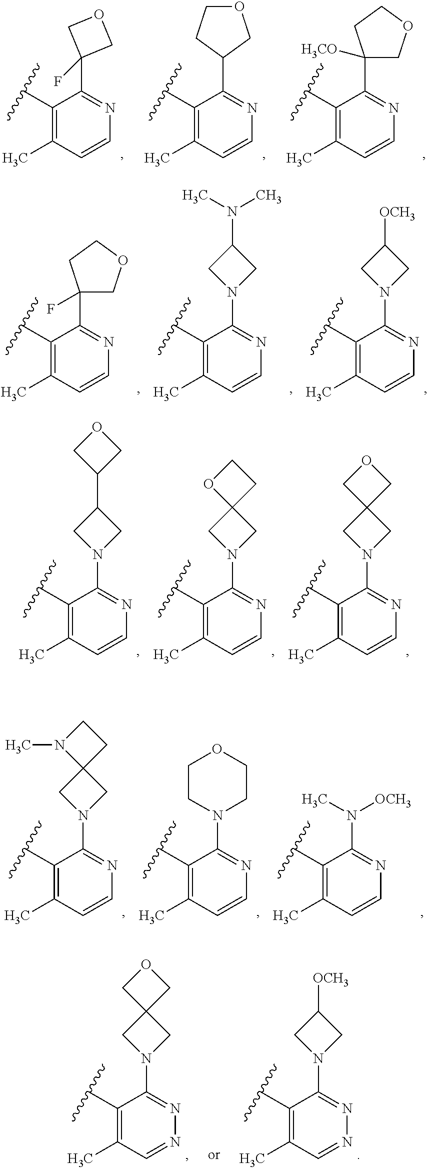

In some cases, Z is

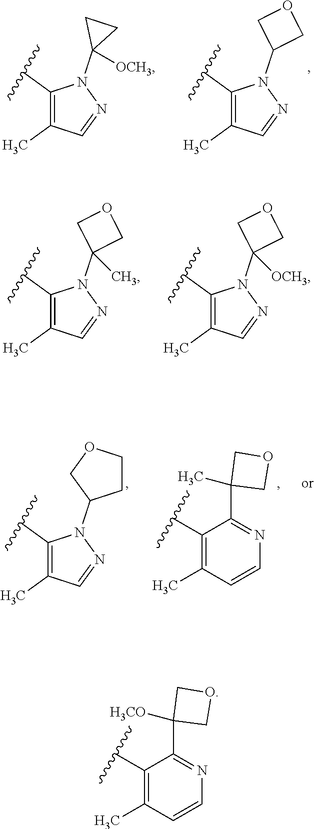

In some cases, Z is a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein the bicyclic ring is optionally substituted with 1-4 substituents, and each substituent independently is halo, CN, C₁₋₆ alkyl, C₁₋₆ haloalkyl, C₀₋₆ alkylene-OH, or C₀₋₆ alkylene-C₁₋₃ alkoxy. In some cases, Z is

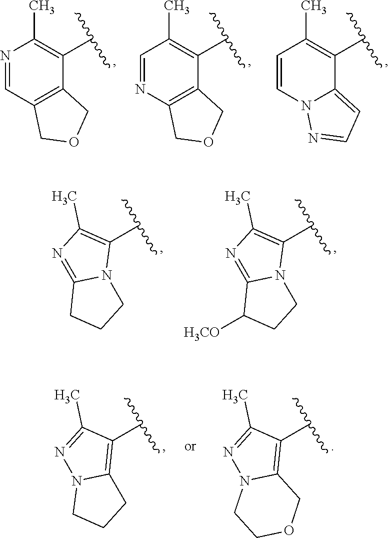

In some cases,

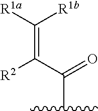

is

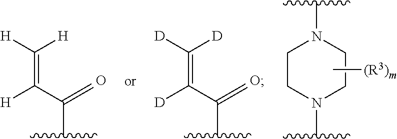

is

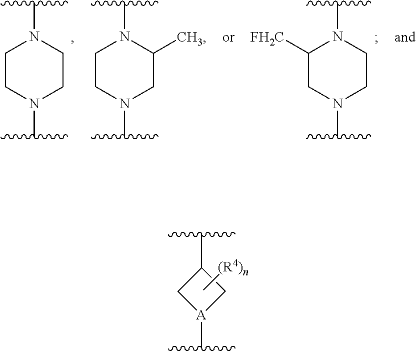

is

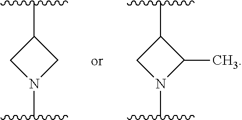 [SMILES: *C1CN(*)C1 ; "or" ; *C1CN(*)C1C]

In some cases,

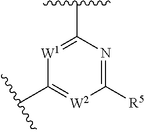

is

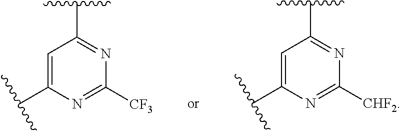 [SMILES: *c1cc(*)nc(C(F)(F)F)n1 ; *c1cc(*)nc(C(F)F)n1 ; "or"]

In some cases, X is

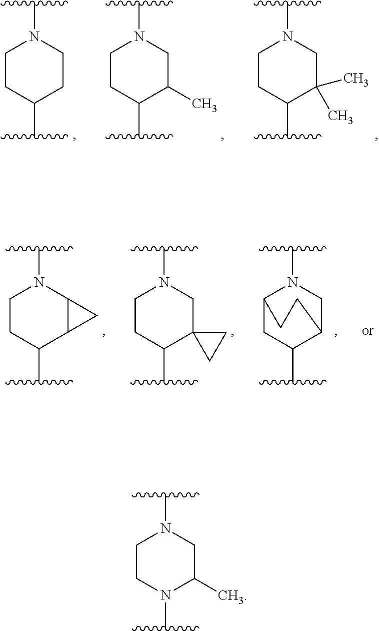

In some cases, Z is

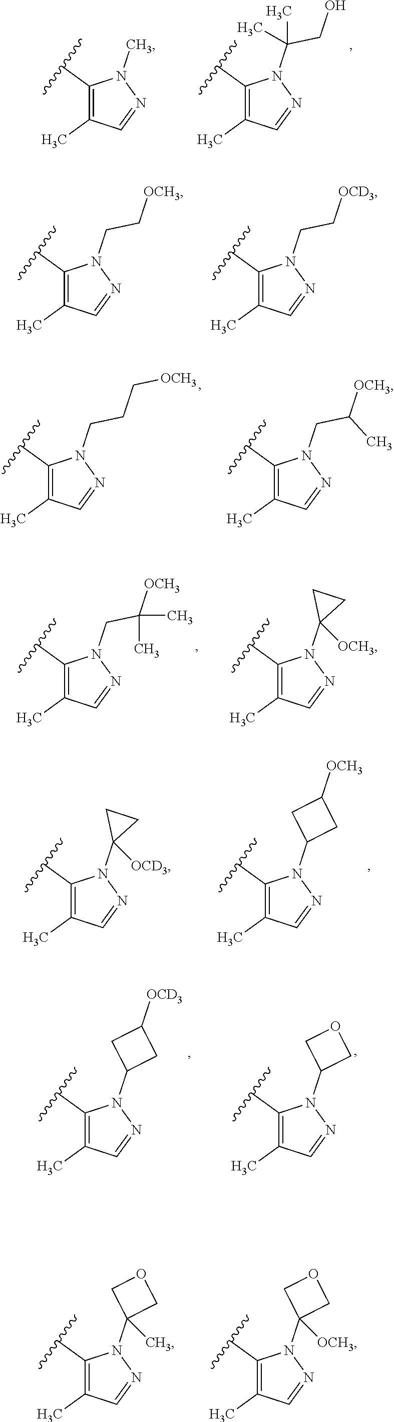

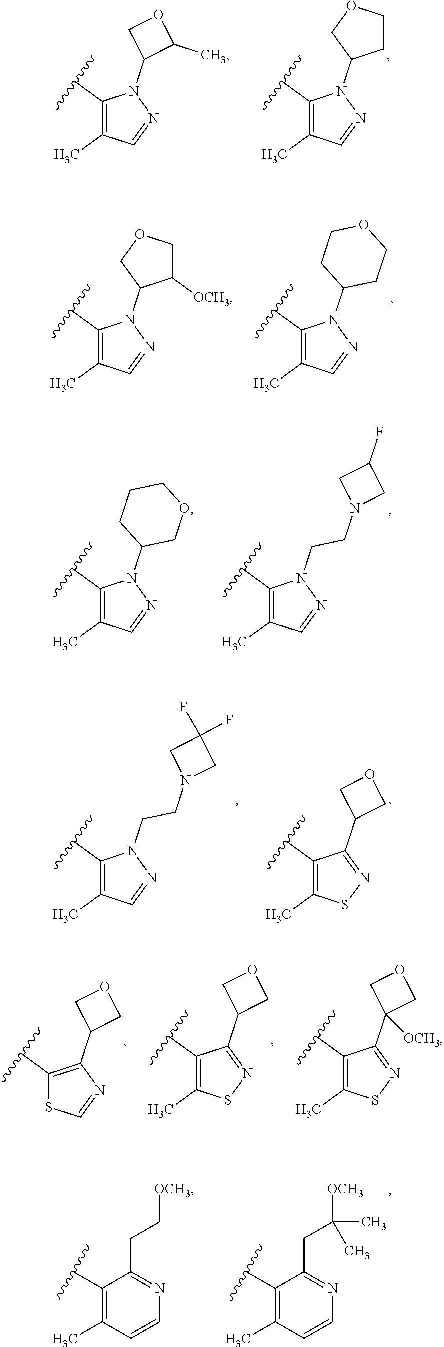

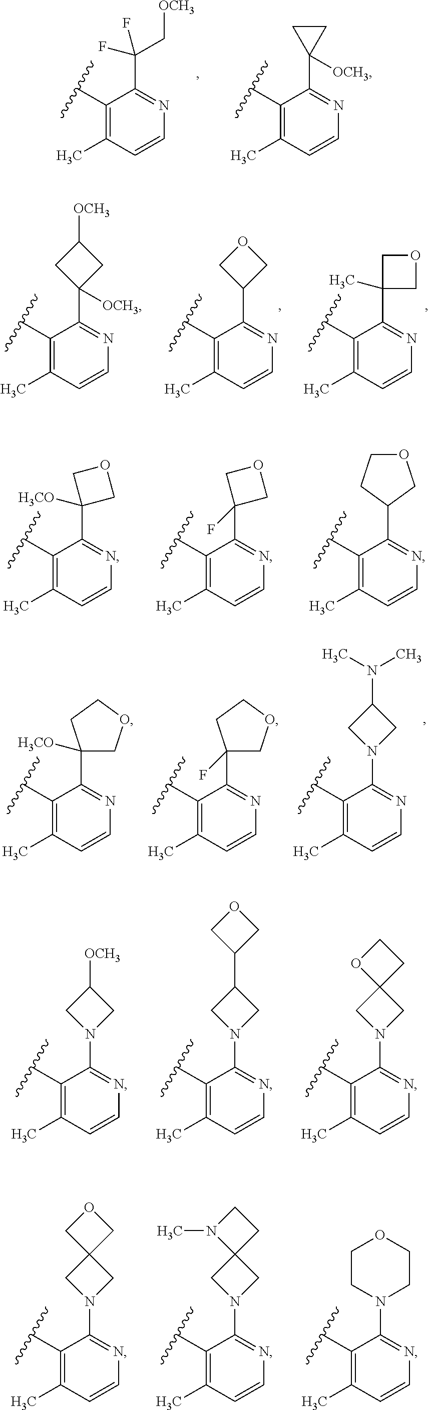

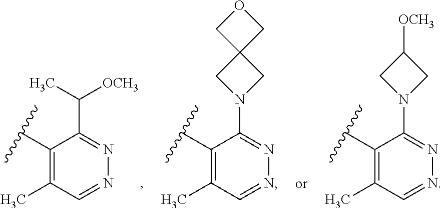 [SMILES: *c1c(C)cnnc1C(C)OC ; *c1c(C)cnnc1N1CC2(COC2)C1 ; *c1c(C)cnnc1N1CC(OC)C1]

In some cases, the compound of Formula (I) is a compound of Formula (I′)

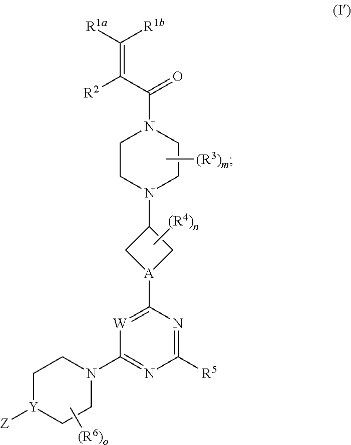

### Formula (IA):

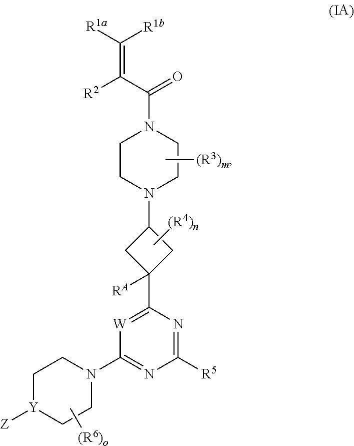

wherein R^{A} is H, halo, CN, C₁₋₃ alkyl, C₁₋₃ haloalkyl, C₁₋₃ alkyleneOH, or C₀₋₃ alkylene-C₁₋₄ alkoxy; Formula (IB):

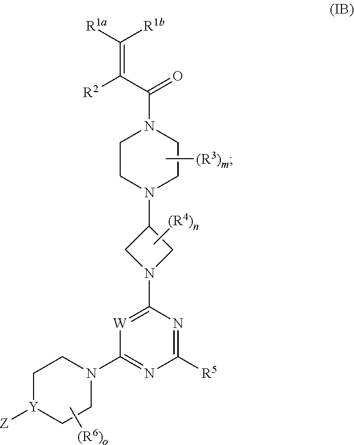

### Formula (IC)

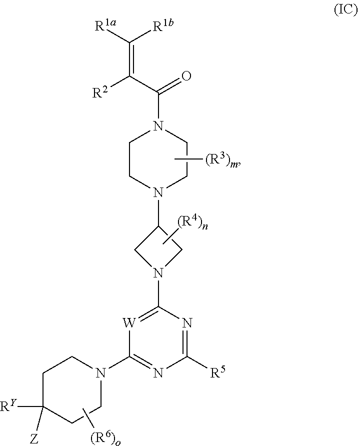

wherein R^{Y} is H, halo, CN, C₁₋₃ alkyl, C₁₋₃ haloalkyl, C₀₋₃ alkyleneOH, or C₀₋₃ alkylene-C₁₋₄ alkoxy; Formula (ID):

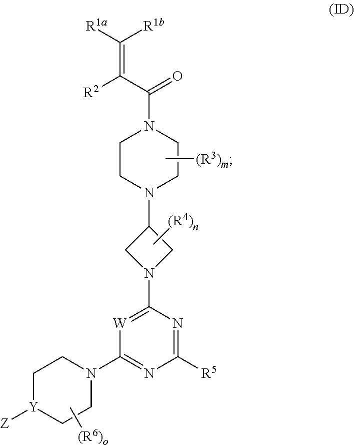

### Formula (IE):

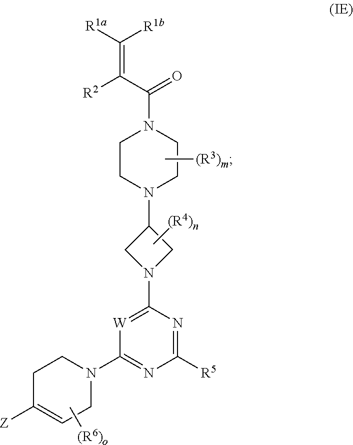

### or Formula (IF):

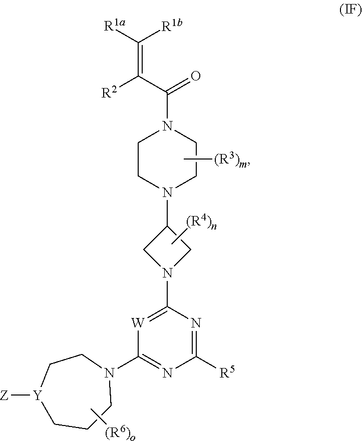

or a pharmaceutically acceptable salt of any of the foregoing. In some cases, the compound of Formula (I) is a compound listed in Table A, or a pharmaceutically acceptable salt thereof. In some cases, the compound of Formula (I) is a compound listed in Table E, or a pharmaceutically acceptable salt thereof.

Another aspect of the disclosure provides a pharmaceutical composition comprising a compound of the disclosure (e.g., a compound of Formula (I), a compound of Formula (I′), a compound of Formula (IA), a compound of Formula (IB), a compound of Formula (IC), a compound of Formula (ID), a compound of Formula (IE), a compound of Formula (IF), a compound listed in Table A, a compound listed in Table B, a compound listed in Table C, a compound listed in Table D, or a compound listed in Table E), or a pharmaceutically acceptable salt of any of the foregoing, and a pharmaceutically acceptable excipient.

A further aspect of the disclosure provides a compound of the disclosure (e.g., a compound of Formula (I), a compound of Formula (I), a compound of Formula (IA), a compound of Formula (IB), a compound of Formula (IC), a compound of Formula (ID), a compound of Formula (IE), a compound of Formula (IF), a compound listed in Table A, a compound listed in Table B, a compound listed in Table C, a compound listed in Table D, or a compound listed in Table E), or a pharmaceutically acceptable salt of any of the foregoing, or a pharmaceutical composition of the disclosure, for use as a medicament.

Yet another aspect of the disclosure provides a compound of the disclosure (e.g., a compound of Formula (I), a compound of Formula (I′), a compound of Formula (IA), a compound of Formula (IB), a compound of Formula (IC), a compound of Formula (ID), a compound of Formula (IE), a compound of Formula (IF), a compound listed in Table A, a compound listed in Table B, a compound listed in Table C, a compound listed in Table D, or a compound listed in Table E), or a pharmaceutically acceptable salt of any of the foregoing, or a pharmaceutical composition of the disclosure for use in treating cancer. In some cases, the cancer is characterized by one or more cells expressing KRAS^{G12C} mutant protein. In some cases, the cancer is non-small cell lung cancer, small bowel cancer, appendiceal cancer, colorectal cancer, cancer of unknow n primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Still another aspect of the disclosure provides use of a compound of the disclosure (e.g., a compound of Formula (I), a compound of Formula (I′), a compound of Formula (IA), a compound of Formula (IB), a compound of Formula (IC), a compound of Formula (ID), a compound of Formula (IE), a compound of Formula (IF), a compound listed in Table A, a compound listed in Table B, a compound listed in Table C, a compound listed in Table D, or a compound listed in Table E), or a pharmaceutically acceptable salt of any of the foregoing, or a pharmaceutical composition of the disclosure in the preparation of a medicament for treating cancer. In some cases, the cancer is characterized by one or more cells expressing KRAS^{G12C} mutant protein. In some cases, the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic, myeloproliferative neoplasms, head and neck cancer, esophagogastric cancer, soft tissue sarcoma, mesothelioma, thyroid cancer, leukemia, melanoma, or a solid tumor.

Another aspect of the disclosure provides a method of treating cancer in a subject in need thereof, the method comprising administering to the subject a therapeutically effective amount of the compound of the disclosure (e.g., a compound of Formula (I), a compound of Formula (I′), a compound of Formula (IA), a compound of Formula (IB), a compound of Formula (IC), a compound of Formula (ID), a compound of Formula (IE), a compound of Formula (IF), a compound listed n Table A, a compound listed in Table B, a compound listed in Table C, a compound listed in Table D, or a compound listed in Table E), or a pharmaceutically acceptable salt of any of the foregoing, or a pharmaceutical composition of the disclosure. In some cases, the cancer is characterized by one or more cells expressing KRAS^{G12C} mutant protein. In some cases, the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor. In some cases, the subject has a cancer that was determined to have one or more cells expressing the KRAS^{G12C} mutant protein prior to administration of the compound, salt, or pharmaceutical composition. In some cases, the method further comprises simultaneous, separate, or sequential administration of an effective amount of a second compound. In some cases, the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents.

Further aspects and advantages will be apparent to those of ordinary skill in the art from a review of the following detailed description. The description hereafter includes specific cases, embodiments and examples with the understanding that the disclosure is illustrative and is not intended to limit the embodiments of the present disclosure to the specific cases, embodiments, and examples described herein.

### DETAILED DESCRIPTION

Disclosed herein are compounds having activity as inhibitors of the G12C-mutant KRAS protein, pharmaceutical compositions comprising the compounds, and uses and methods of treating disorders, such as cancer, with the compounds and pharmaceutical composition described herein.

### Compounds of the Disclosure

Provided herein are compounds of Formula (I).

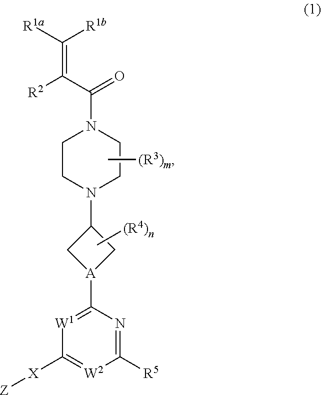

and pharmaceutically acceptable salts thereof, wherein:
wherein:
m is 0, 1, 2, 3, or 4;
n is 0, 1, or 2;
A is N, CH, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy;
each of W¹ and W² independently is N, CH, C-halo, C—CN, C—C₁₋₃ alkyl, C—C₂₋₃ alkenyl, C—C₂₋₃ alkynyl, C—C₁₋₃ haloalkyl, C—C₀₋₃ alkyleneOH, or C—C₀₋₃ alkylene-C₁₋₄ alkoxy, wherein each of the alkenyl and alkynyl is optionally substituted with 1 or more substituents;
X is heterocycloalkyl or heterocycloalkenyl, each having 4-7 total ring atoms and 1-3 heteroatoms selected from N, O, and S, wherein each of the heterocycloalkyl and heterocycloalkenyl is optionally substituted with 1 or more substituents;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein each of the phenyl, heteroaryl, and bicyclic rings is optionally substituted with 1 or more substituents;
each of R^{1a}, R^{1b}, and R² independently is H, D, halo, C₁₋₄ alkyl, C₁₋₄ haloalkyl, C₁₋₂ alkylene-OH, C₀₋₂ alkylene-C₁₋₄ alkoxy, C₀₋₂ alkylene-C₁₋₄ haloalkoxy, C₀₋₂ alkylene-CN, C₀₋₂ alkylene-N(R^{N1})₂, C₁₋₂ alkylene-heterocycloalkyl having 3-6 total ring atoms and 1 or 2 heteroatoms selected from N, O, and S, or R^{1b} and R², together with the carbon atoms to which they are attached, form

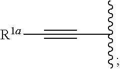

each R³ independently is C₁₋₃ alkyl, C₁₋₃ haloalkyl,

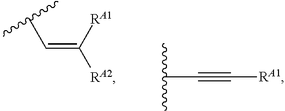

C₀₋₃ alkyleneCN, C₀₋₃ alkyleneOH, C₀₋₃ alkylene-C₁₋₃ alkoxy, oxo, spiro-cycloalkyl having 3-7 total ring atoms, spiro-cycloalkenyl having 4-7 total ring atoms, spiro-heterocycloalkyl having 3-7 total ring atoms and 1 or 2 heteroatoms selected from N, O and S, spiro-heterocycloalkenyl having 4-7 total ring atoms and 1 or 2 heteroatoms selected from N, O and S, or two adjacent R³, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1 or 2 heteroatoms selected from N, O, and S, or a fused heterocycloalkenyl ring having 4-7 total ring atoms and 1 or 2 heteroatoms selected from N, O, and S;
each R⁴ independently is C₁₋₃ alkyl, C₁₋₃ haloalkyl, C₀₋₃ alkyleneCN, C₁₋₃ alkyleneOH, C₁₋₃ alkylene-C₁₋₃ alkoxy, oxo, spiro-cycloalkyl having 3-7 total ring atoms, or spiro-heterocycloalkyl having 4-7 total ring atoms and 1 or 2 heteroatoms selected from N, O and S;
R⁵ is C₁₋₃ haloalkyl, C₁₋₆ alkyl, C₂₋₄ alkenyl, C₂₋₄ alkynyl, halo, C₁₋₃ alkoxy, C₁₋₃ thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing independently is optionally substituted with 1 or more substituents;
each of R^{A1} and R^{A2} independently is H, C₁₋₃ alkyl, C₁₋₃ haloalkyl, or cycloalkyl having 3-5 total ring atoms; and
each R^{N1} independently is H or C₁₋₄ alkyl.

In some cases, R^{1a} is H or D. In some cases, R^{1a} is H. In some cases, R^{1a} is D. In some cases, R^{1b} is H or D. In some cases, R^{1b} is H. In some cases, R^{1b} is D. In some cases, R² is H or D. In some cases, R² is H. In some cases, R² is D. In some cases, at least one of R^{1a}, R^{1b}, and R² is H or D. In some cases, at least one of R^{1a}, R^{1b}, and R² is H. In some cases, at least one of R^{1a}, R^{1b}, and R² is D. In some cases, at least two of R^{1a}, R^{1b}, and R² are each independently H or D. In some cases, at least two of R^{1a}, R^{1b}, and R² are H. In some cases, at least two of R^{1a}, R^{1b}, and R² are D. In some cases, each of R^{1a}, R^{1b}, and R² independently is H or D. In some cases, each of R^{1a}, R^{1b}, and R² independently is H. In some cases, each of R^{1a}, R^{1b}, and R² independently is D. In some cases, two of R^{1a}, R^{1b}, and R² are H and one of R^{1a}, R^{1b}, and R² is halo, C₁₋₄ alkyl, C₁₋₄ haloalkyl, C₁₋₂ alkylene-OH, C₀₋₂ alkylene-C₁₋₄ alkoxy, C₀₋₂ alkylene-C₁₋₄ haloalkoxy, C₀₋₂ alkylene-CN, C₀₋₂ alkylene-N(R^{N1})₂, or C₁₋₂ alkylene-heterocycloalkyl having 3-6 total ring atoms and 1 or 2 heteroatoms selected from N, O, and S. In some cases, at least one of R^{1a}, R^{1b}, and R² is halo. In some cases, one of R^{1a}, R^{1b}, and R² is halo. In some cases, R^{1a} is halo and each of R^{1b} and R² is H. In some cases, at least one of R^{1a}, R^{1b}, and R² is Br, Cl, or F. In some cases, one of R^{1a}, R^{1b}, and R² is Br, Cl, or F. In some cases, R^{1a} is Br, Cl, or F and each of R^{1b} and R² is H. In some cases, at least one of R^{1a}, R^{1b}, and R² is Br or Cl. In some cases, one of R^{1a}, R^{1b}, and R² is Br or Cl. In some cases, R^{1a} is Br or Cl and each of R^{1b} and R² is H. In some cases, at least one of R^{1a}, R^{1b}, and R² is C₁₋₄ alkyl or C₁₋₄ haloalkyl. In some cases, one of R^{1a}, R^{1b}, and R² is C₁₋₄ alkyl or C₁₋₄ haloalkyl. In some cases, at least one of R^{1a}, R^{1b}, and R² is CH₃, CH₂CH₃, CH₂CH₂CH₃, CH(CH₃)₂, CH₂CH₂CH₂CH₃, CH₂F, CHF₂, or CF₃. In some cases, one of R^{1a}, R^{1b}, and R² is CH₃, CH₂CH₃, CH₂CH₂CH₃, CH(CH₃)₂, CH₂CH₂CH₂CH₃, CH₂F, CHF₂, or CF₃. In some cases, at least one of R^{1a}, R^{1b}, and R² is CH₃, CH₂F, CHF₂, or CF₃. In some cases, one of R^{1a}, R^{1b}, and R² is CH₃, CH₂F, CHF₂, or CF₃. In some cases, at least one of R^{1a}, R^{1b}, and R² is CH₃ or CF₃. In some cases, one of R^{1a}, R^{1b}, and R² is CH₃ or CF₃. In some cases, at least one of R^{1a}, R^{1b}, and R² is C₁₋₂ alkylene-OH, C₀₋₂ alkylene-C₁₋₄ alkoxy, C₀₋₂ alkylene-C₃₋₄ haloalkoxy, C₀₋₂ alkylene-CN, or C₀₋₂ alkylene-N(R^{N1})₂, and each R^{N1} independently is H or C₁₋₄ alkyl. In some cases, each R^{N1} independently is H or CH₃. In some cases, each R^{N1} independently is H. In some cases, at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂. In some cases, one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂. In some cases, at least one of R^{1a}, R^{1b}, and R² is C₁₋₂ alkylene-heterocycloalkyl wherein the heterocycloalkyl group contains 3-6 total ring atoms and 1 or 2 heteroatoms selected from N, O, and S. In some cases, the heterocycloalkyl is aziridinyl, oxiranyl, azetidinyl, oxetanyl, pyrrolidinyl, tetrahydrofuranyl, imidazolidinyl, pyrazolidinyl, oxathiolidinyl, isoxthiodinyl, oxazolidinyl, isoxazolidinyl, thiazolidinyl, isothiazolidinyl, piperidinyl, diazinyl, or morpholinyl. In some cases, the heterocycloalkyl is aziridinyl, azetidinyl, pyrrolidinyl, piperidinyl, or morpholinyl. In some cases, at least one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl. In some cases, one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl. In some cases, one of R^{1a}, R^{1b}, and R² is Br, Cl, F, CH₃, CH₂F, CHF₂, CF₃, CH₂OH, OCH₃, CH₂OCH₃, OCF₃, CH₂OCF₃, CN, CH₂CN, NH₂, N(CH₃)₂, CH₂NH₃, CH₂N(CH₃)₂, aziridin-1-yl-methyl, azetidin-1-yl-methyl, pyrrolidine-1-yl-methyl, piperidin-1-yl-methyl, or morpholin-1-yl-methyl. In some cases, R^{1b} and R² together with the carbon atoms to which they are attached form

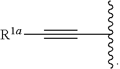

In some cases, R^{1a} is H. In some cases, R^{1b} and R² together with the carbon atoms to which they are attached form

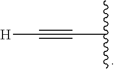 [SMILES: *C#C]

In some cases,

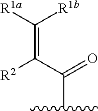

is

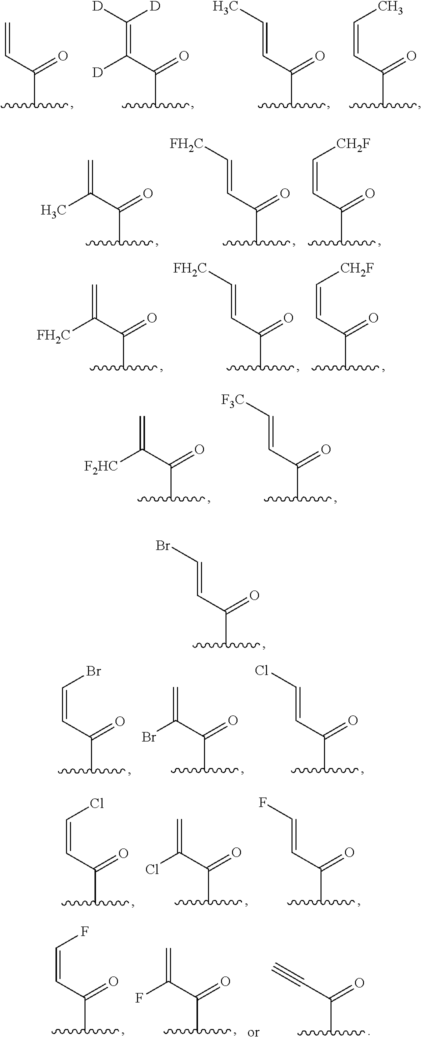

In some cases,

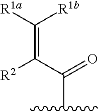

is

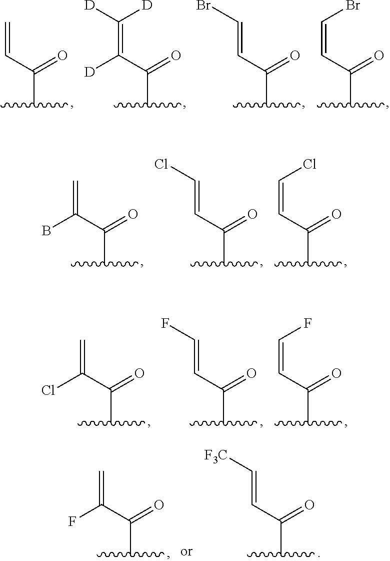

In some cases,

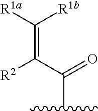

is

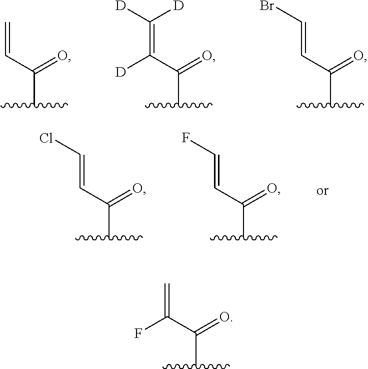

In some cases,

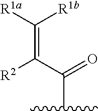

is

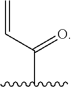 [SMILES: *C(=O)C=C]

In some cases, m is 0, and

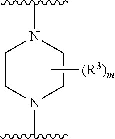

is

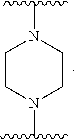 [SMILES: *N1CCN(*)CC1]

In some cases, m is 1. In some cases, m is 2. In some cases, m is 3. In some cases, m is 4. In some cases,

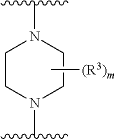

is deuterated. In some cases,

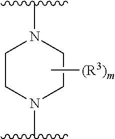

is fully deuterated. In some cases,

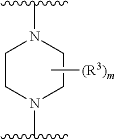

is

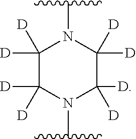

In some cases, at least one R³ is C₁₋₃ alkyl or C₁₋₃ haloalkyl. In some cases, at least one R³ is CH₃, CH₂CH₃, CH₂CH₂CH₃, CH(CH₃)₂, CF₃, CHF₂, or CH₂F. In some cases, at least one R³ is CH₃, CH₂CH₃, CF₃, CHF₂, or CH₂F. In some cases, at least one R³ is CH₃. In some cases, m is 1 or 2 and each R³ is CH₃. In some cases, m is 1 and R³ is CF₃. CHF₂, or CH₂F. In some cases, at least one R³ is

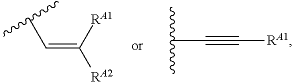

and each of R^{A1} and R^{A2} independently is H, C₁₋₃ alkyl, C₁₋₃ haloalkyl, or cycloalkyl having 3-5 total ring atoms. In some cases, m is 1 and R³ is

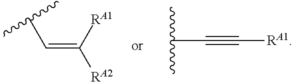

In some cases, each of R^{A1} and R^{A2} independently is H, CH₃, CH₂F, CHF₂, CF₃, CH₂CH₃, CH₂CH₂CH₃, CH(CH₃)₂, cyclopropyl, or cyclobutyl. In some cases,

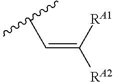

is

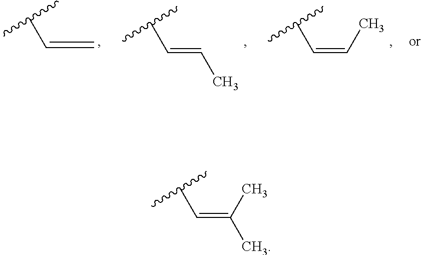

In some cases,

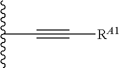

is

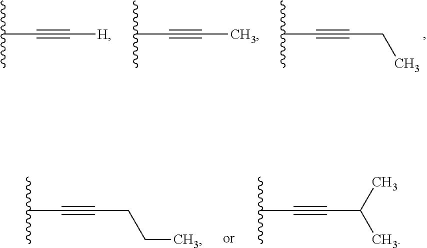

In some cases,

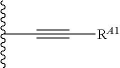

is

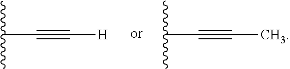

In some cases, at least one R³ is

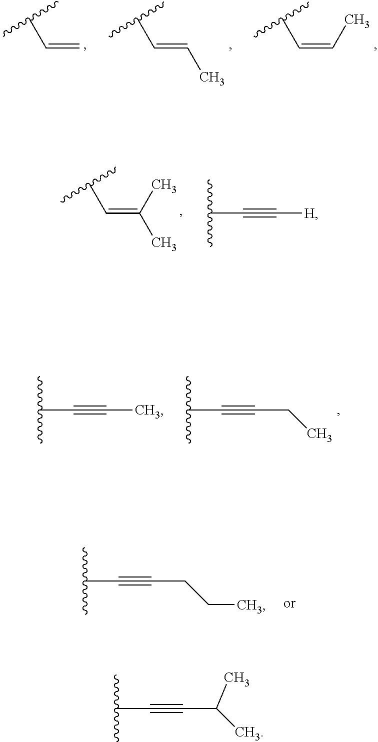

In some cases, at least one R³ is

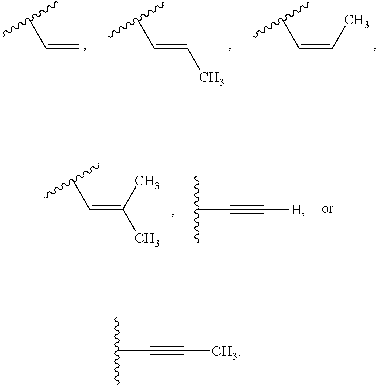

In some cases, at least one R³ is C₀₋₃ alkyleneCN. In some cases, at least one R³ is CN, CH₂CN, or CH₂CH₃CN. In some cases, at least one R³ is CN or CH₃CN. In some cases, m is 1 and R³ is CN or CH₂CN. In some cases, at least one R³ is C₀₋₃ alkyleneOH or C₀₋₃ alkylene-C₁₋₃ alkoxy. In some cases, at least one R³ is OH, CH₂OH, CH₂CH₂OH, OCH₃, CH₂OCH₃, or CH₂CH₂OCH₃. In some cases, m is 1 and R³ is OH, CH₂OH, CH₂CH₂OH, OCH₃, CH₂OCH₃, or CH₂CH₂OCH₃, In some cases, at least one R³ is oxo. In some cases, at least one R³ is spiro-cycloalkyl having 3-7 total ring atoms or spiro-heterocycloalkyl having 3-7 total ring atoms and 1 or 2 heteroatoms selected from N, O and S. In some cases, at least one R³ is spiro-cyclopropyl, spiro-cyclobutyl, spiro-cyclopentyl, spiro-azetidinyl, spiro-oxetanyl, spiro-pyrrolidinyl, spiro-imidazolidinyl, spiro-pyrazolidinyl, or spiro-tetrahydrofuranyl. In some cases, at least one R³ is spiro-cyclopropyl, spiro-cyclobutyl, spiro-oxetanyl, or spiro-tetrahydrofuranyl. In some cases, m is 1 and R³ is spiro-cyclopropyl or spiro-oxetan %1. In some cases, at least one R³ is spiro-cycloalkenyl having 4-7 total ring atoms or spiro-heterocycloalkenyl having 4-7 total ring atoms and 1 or 2 heteroatoms selected from N, O and S. In some cases, two adjacent R³, together with the atoms to which the % are attached, form a fused cycloalkyl ring having 3-7 total ring atoms or a fused heterocycloalkyl ring having 3-7 total atoms and 1 or 2 heteroatoms selected from N, O, and S. In some cases, two adjacent R³, together with the atoms to which they are attached, form a fused cycloalkyl ring having 3-7 total ring atoms. In some cases, two adjacent R³, together with the atoms to which they are attached, form a fused-cyclopropyl ring, a fused-cyclobutyl ring, a fused-cyclopentyl ring, or a fused-cyclohexyl ring. In some cases, two adjacent R³, together with the atoms to which they are attached, form a fused-cyclopropyl ring or a fused-cyclobutyl ring. In some cases, two adjacent R³, together with the atoms to which they are attached, form a fused cycloalkenyl ring having 4-7 total ring atoms or a fused heterocycloalkenyl ring having 4-7 total ring atoms and 1 or 2 heteroatoms selected from N, O and S. In some cases, each R³ independently is CH₃, CH₂CH₃, CF₃, CHF₂, CH₂F, CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, spiro-tetrahydrofuranyl, fused-cyclopropyl, or fused-cyclobutyl. In some cases, each R³ independently is CH₃, CH₂CH₃, CF₃, CHF₂, CH₂F,

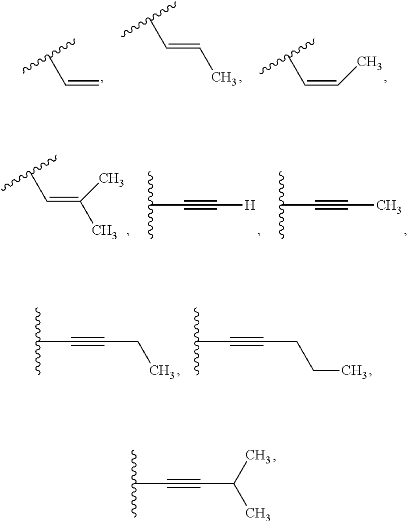

CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl, or two adjacent R³, together with the atoms to which they are attached, form a fused cyclopropyl ring or a fused cyclobutyl ring. In some cases, each R³ independently is CH₃, CH₂CH₃, CF₃, CHF₂, CH₂F, CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl. In some cases, m is 1 and R³ is CH₃, CF₃, CHF₂, CH₂F, CN, CH₂CN, CH₂OH, CH₂OCH₃, or spiro-oxetanyl. In some cases,

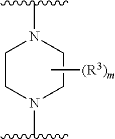

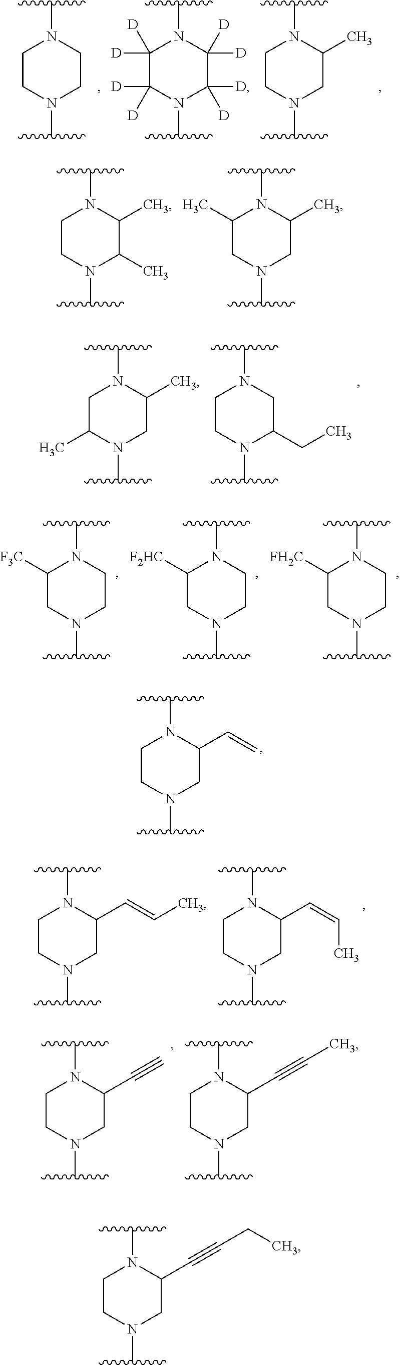

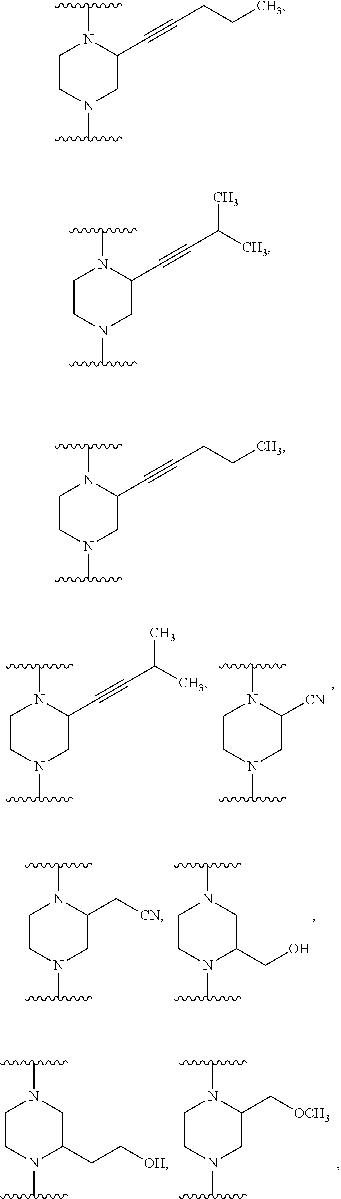

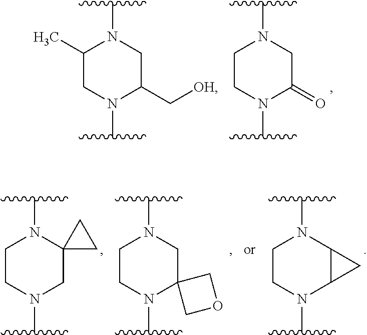

In some cases,

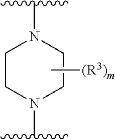

is

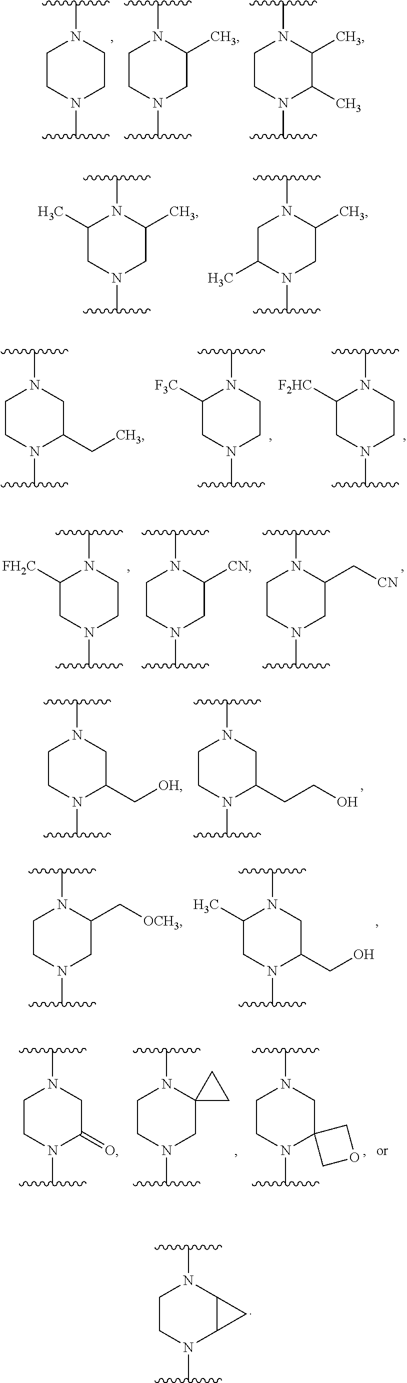

In some cases,

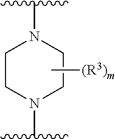

is

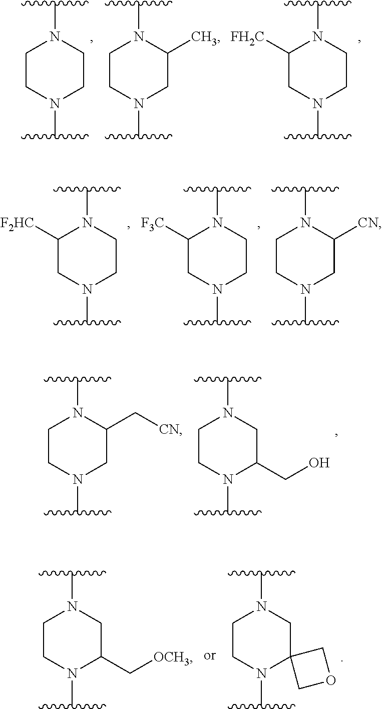

In some cases,

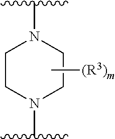

is

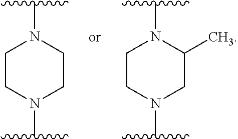 [SMILES: *N1CCN(*)CC1 ; *N1CCN(*)C(C)C1]

In some cases,

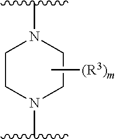

is

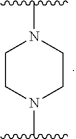 [SMILES: *N1CCN(*)CC1]

In some cases,

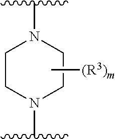

is

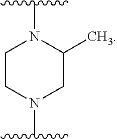 [SMILES: *N1CCN(*)C(C)C1]

In some cases, A is N, CH, or C—C₁₋₃ alkyl. In some cases, A is N. In some cases, A is CH, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy. In some cases, A is CH. In some cases, A is C-halo or C—CN. In some cases, A is C—F or C—Cl. In some cases, A is C—F. In some cases, A is C—CN. In some cases, A is C—C₁₋₃ alkyl or C—C₁₋₃ haloalkyl. In some cases, A is C—CH₃, C—CH₂CH₃, C—CH₂CH₂CH₃, C—CH(CH₃)₂, C—CF₃, C—CHF₂, or C—CH₂F. In some cases, A is C—CH₃, C—CH₂F, C—CHF₂, or C—CF₃. In some cases, A is C—CH₃. In some cases, A is C—CH₂F, C—CHF₂, or C—CF₃. In some cases, A is C—C₀₋₃ alkyleneOH or C—C₀₋₃ alkylene-C₁₋₄ alkoxy. In some cases, A is C—OH, C—CH₂OH, C—CH₂CH₂OH, C—OCH₃, C—CH₂OCH₃, or C—CH₂CH₂OCH₃. In some cases, A is C—OH, C—CH₂OH, C—OCH₃, or C—CH₂OCH₃. In some cases, A is CH, C—F, C—Cl, C—CN, C—CH₃, C—CH₂F, C—CHF₂, C—CF₃, C—OH, C—CH₂OH, C—OCH₃, or C—CH₂OCH₃. In some cases, A is N, CH, C—F, C—Cl, C—CN, C—CH₃, C—CH₂CH₃, C—CH₂CH₂CH, C—CH(CH₃)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, A is N, CH, C—F, C—Cl, C—CN, C—CH₃, C—CF₃, C—CHF₂, C—CH₂F, C—OH, C—CH₂OH, C—OCH₃, or C—CH₂OCH₃. In some cases, A is N, CH, or C—CH₃. In some cases, n is 0. In some cases, n is 1. In some cases, n is 2. In some cases, at least one R⁴ is C₁₋₃ alkyl or C₁₋₃ haloalkyl. In some cases, at least one R⁴ is CH₃, CH₂CH₃, CH₃CH₂CH₃, CH(CH₃)₂, CF₃, CHF₂, or CH₂F. In some cases, at least one R⁴ is CH₃. In some cases, one R⁴ is CH₃, CH₂CH₃, CH₂CH₂CH₃, CH(CH₃)₂, CF₃, CHF₂, or CH₂F. In some cases, n is 2 and each R⁴ independently is CH₃, CH₂CH₃, CH₂CH₂CH₃, CH(CH₃)₂, CF₃, CHF₂, or CH₂F. In some cases, n is 1 and R⁴ is CH₃, CH₂CH₃, CH₂CH₂CH₃, CH(CH₃)₂, CF₃, CHF₂, or CH₂F. In some cases, n is 1 and R⁴ is CH₃. In some cases, at least one R³ is C₀₋₃ alkyleneCN. In some cases, at least one R⁴ is CN or CH₂CN. In some cases, n is 1 and R⁴ is CN or CH₂CN. In some cases, at least one R⁴ is C₁₋₃ alkyleneOH or C₁₋₃ alkylene-C₁₋₃ alkoxy. In some cases, at least one R³ is CH₂OH, CH₂CH₂OH, OCH₃, CH₂OCH₃, or CH₂CH₂OCH₃. In some cases, n is 1 and R³ is CH₂OH, CH₂CH₂OH, OCH₃, CH₂OCH₃, or CH₂CH₂OCH₃. In some cases, at least one R⁴ is oxo. In some cases, at least one R³ is spiro-cycloalkyl having 3-7 total ring atoms. In some cases, at least one R⁴ is spiro-cyclopropyl, spiro-cyclobutyl, or spiro-cyclopentyl. In some cases, n is 1 and R⁴ is spiro-cyclopropyl or spiro-cyclobutyl. In some cases, at least one R³ is spiro-heterocycloalkyl having 4-7 total ring atoms and 1 or 2 heteroatoms selected from N, O and S. In some cases, at least one R³ is spiro-oxetanyl or spiro-tetrahydrofuranyl. In some cases, n is 1 and R⁴ is spiro-oxetanyl. In some cases, each R³ independently is CH₃, CH₂CH₃, CH₂CH₂CH₃, CH(CH₃)₂, CF₃, CHF₂, CH₂F, CN, CH₂CN, CH₂OH, CH₂CH₂OH, OCH₃, CH₂OCH₃, CH₂CH₂OCH₃, oxo, spiro-cyclopropyl, spiro-cyclobutyl, or spiro-cyclopentyl. In some cases, each R³ independently is CH₃, CH₂CH₃, CH₂CH₂CH₃, CH(CH₃)₂, CF₃, CHF₂, CH₂F, CN, CH₂CN, CH₂OH, CH₂CH₂OH, CH₂OCH₃, CH₂CH₂OCH₃, oxo, spiro-cyclopropyl, spiro-cyclobutyl, or spiro-oxetanyl. In some cases, each R³ independently is CH₃, CH₂CH₃, CH₂CH₂CH₃, CH₂F, CN, CH₂CN. CH₂OH, CH₂CH₂OH, CH₂OCH₃, spiro-cyclopropyl, or spiro-oxetanyl. In some cases,

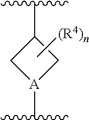

is

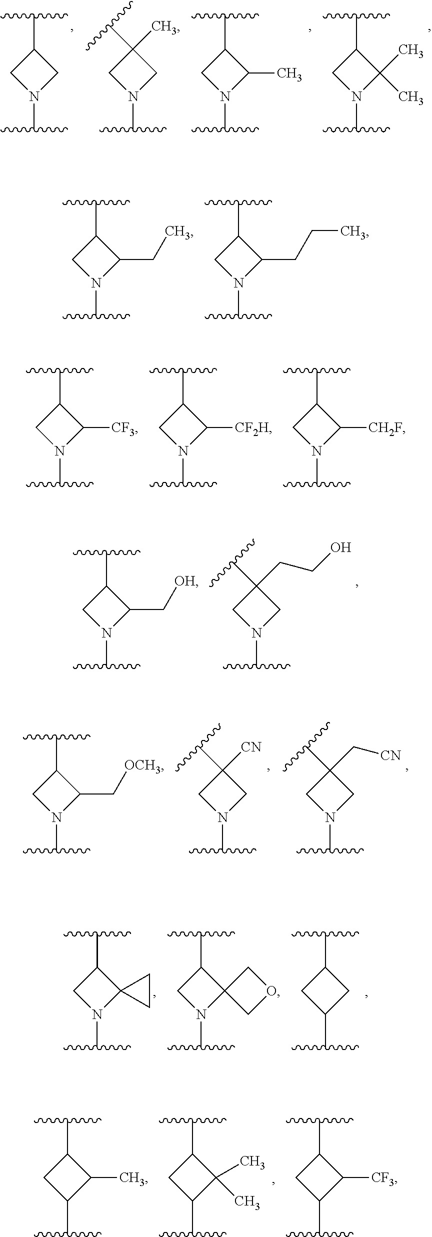

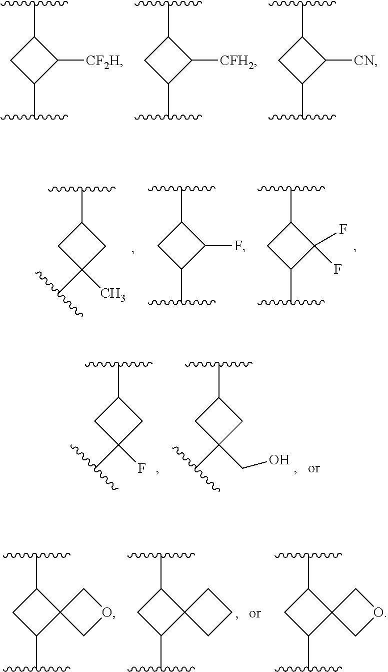

In some cases,

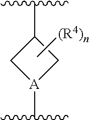

is

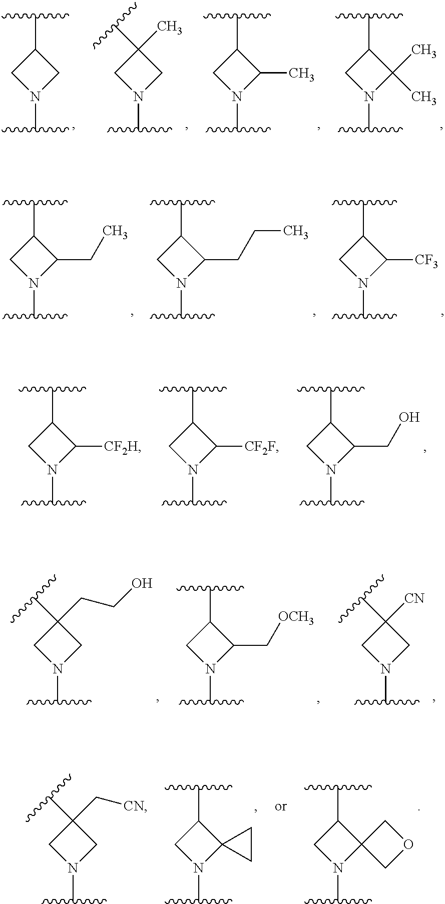

In some cases,

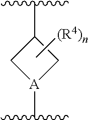

is

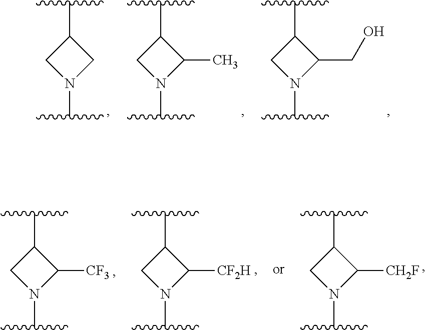

In some cases,

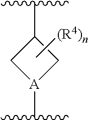

is

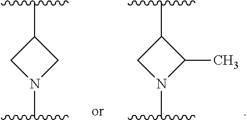

In some cases,

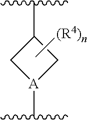

is

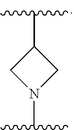 [SMILES: *C1CN(*)C1]

In some cases,

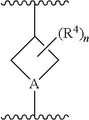

is

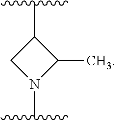 [SMILES: *C1CN(*)C1C]

In some cases,

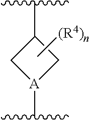

is

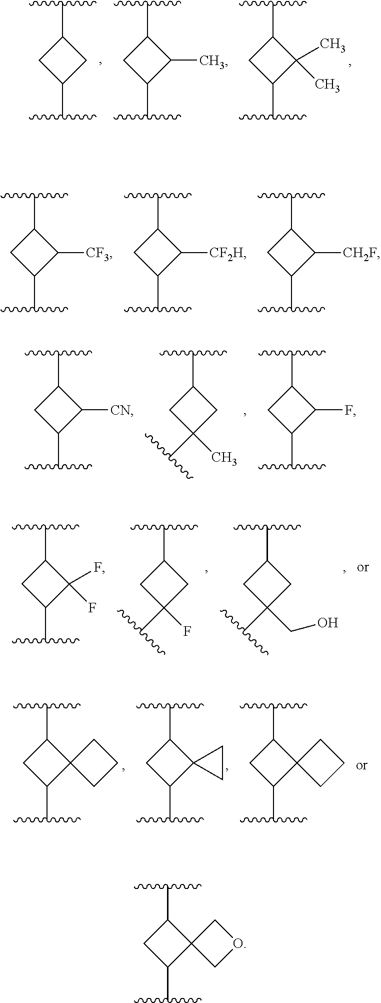

In some cases,

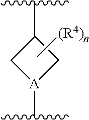

is

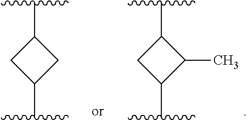

In some cases, W¹ is N. In some cases, W¹ is CH. In some cases, W¹ is C-halo or C—CN. In some cases, W¹ is C—Br, C—Cl, or C—F. In some cases, W¹ is C—F, C—Cl, or C—CN. In some cases, W¹ is C—C₁₋₃ alkyl or C—C₁₋₃ haloalkyl. In some cases, W¹ is C—CH₃, C—CH₂CH₃, C—CH₂CH₂CH₃, C—CH(CH₃)?, C—CF₃, C—CHF₂, or C—CH₂F. In some cases, W¹ is C—CH₃, C—CH₂CH₃, C—CH₂F, C—CHF₂, or C—CF₃. In some cases. W¹ is C—CH₃ or C—CH₂CH₃. In some cases, W¹ is C—C₂₋₃ alkenyl or C—C₂₋₃ alkynyl, and each of the alkenyl and alkynyl is optionally substituted with 1 or more substituents. In some cases, each of the alkenyl and alkynyl is unsubstituted. In some cases, each of the alkenyl and alkynyl is substituted with 1-3 substituents, and each substituent independently is halo, C₁₋₃ haloalkyl, C₀₋₃ alkyleneOH, or C₀₋₃ alkyleneC₁₋₄ alkoxy. In some cases, W¹ is C—CH═CH₂, C—C(OH)═CH₂, C—CH═CH(OH), or C—CCH. In some cases, W¹ is C—C₀₋₃ alkyleneOH or C—C₀₋₃ alkylene-C₁₋₄ alkoxy. In some cases, W¹ is C—OH, C—CH₂OH, C—CH₂CH₂OH, C—OCH₃, C—CH₂OCH₃, or C—CH₂CH₂OCH₃. In some cases, W¹ is C—OH, C—CH₂OH, C—OCH₃, or C—CH₂OCH₃, In some cases, W¹ is CH, C—F, C—Cl, C—CN, C—CH₃, C—CH₂CH₃, C—CH₂CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, W¹ is CH, C—F, C—Cl, C—CN, C—CH₃, C—CH₂CH₃, C—OH, C—CH₂OH, C—OCH₃, or C—CH₂OCH₃. In some cases, W¹ is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C—CH₂OH, C—OCH₃, or C—CH₂OCH₃, In some cases, W² is N. In some cases, W² is CH. In some cases, W² is C-halo or C—CN. In some cases, W² is C—F, C—Cl, or C—Br. In some cases, W² is C—F, C—Cl, or C—CN. In some cases, W² is C—C₁₋₃ alkyl or C—C₁₋₃ haloalkyl. In some cases, W² is C—CH₃, C—CH₂CH₃, C—CH₂CH₂CH₃, C—CH(CH₃), C—CF₃, C—CHF₂, or C—CH₂F. In some cases, W² is C—CH₃, C—CH₂CH₃, C—CH₂F, C—CHF₂, or C—CF₃. In some cases, W² is C—CH₃ or C—CH₂CH₃. In some cases, W² is C—C₂₋₃ alkenyl or C—C₂₋₃ alkynyl, and each of the alkenyl and alkynyl is optionally substituted with 1 or more substituents. In some cases, each of the alkenyl and alkynyl is unsubstituted. In some cases, each of the alkenyl and alkynyl is substituted with 1-3 substituents, and each substituent independently is halo, C₁₋₃ haloalkyl, C₀₋₃ alkyleneOH, or C₀₋₃ alkyleneC₁₋₄ alkoxy. In some cases, W² is C—CH═CH₂, C—C(OH)═CH₂, C—CH═CH(OH), or C—CCH. In some cases, W² is C—C₀₋₃ alkyleneOH or C—C₀₋₃ alkylene-C₁₋₄ alkoxy. In some cases, W¹ is C—OH, C—CH₂OH, C—CH₂CH₂OH, C—OCH₃, C—CH₂OCH₃, or C—CH₂CH₂OCH₃. In some cases, W² is C—OH, C—CH₂OH, C—OCH₃, or C—CH₂OCH₃. In some cases, W² is CH, C—F, C—Cl, C—CN, C—CH₃, C—CH₂CH₃, C—CH₂CH₂CH₃, C—CH(CH)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, W² is CH, C—F, C—Cl, C—CN, C—CH₃, C—CH₂CH₃, C—OH, C—CH₂OH, C—OCH₃, or C—CH₂OCH₃. In some cases, W is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C—CH₂OH, C—OCH₃, or C—CH₂OCH₃. In some cases, each of W¹ and W² independently is N, CH, or C—CH₃. In some cases, W¹ is CH and W² is N, CH, or C—CH₃. In some cases, W² is N and W¹ is N, CH, or C—CH₃. In some cases, W¹ is CH and W² is N. In some cases,

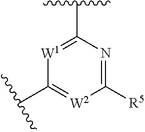

is

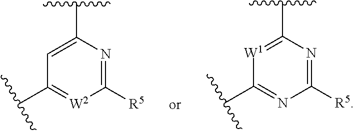

In some cases,

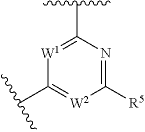

is

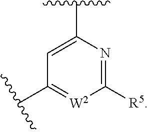

In some cases,

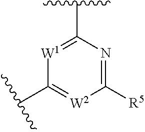

is

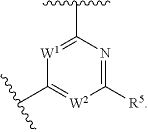

In some cases,

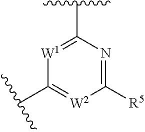

is

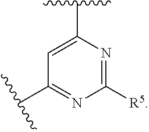

In some cases, R⁵ is C₁₋₃ haloalkyl. In some cases, R⁵ is CF₃, CF₂H, CFH₂, or CF₂CH₃. In some cases, R⁵ is CF₃, CF₂H, or CFH₂. In some cases, R⁵ is CF₃ or CF₂H. In some cases, R⁵ is CF₃. In some cases, R⁵ is CF₂H. In some cases, R⁵ is CHF₂. In some cases, R⁵ is halo. In some cases, R⁵ is Br, Cl, or F. In some cases, R⁵ is C₁₋₃ alkoxy or C₁₋₃ thioalkoxy. In some cases, R⁵ is OCH₃, OCH₂CH₃, SCH₃, or SCH₂—CH₃. In some cases, R³ is OCH₃, or SCH₃. In some cases, R⁵ is C₁₋₆ alkyl, C₂₋₄ alkenyl, or C₂₋₄ alkynyl, wherein each of the alkyl, alkenyl, and alkynyl is optionally substituted with 1, 2, or 3 substituents. In some cases, the C₁₋₆ alkyl is CH₃, CH₂CH₃, CH₂CH₂CH₃, or CH(CH₃)₂, wherein each of the foregoing is optionally substituted with 1 or more substituents. In some cases, the C₂₋₄ alkenyl is CH═CH₂ or CH═CHCH₃, wherein each of the foregoing is optionally substituted with 1 or more substituents. In some cases, the C₂₋₄ alkynyl is

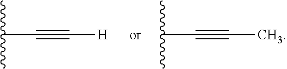

wherein each of the foregoing is optionally substituted with 1 or more substituents. In some cases, the C₁₋₆ alkyl, C₂₋₄ alkenyl, and C₂₋₄ alkynyl is unsubstituted. In some cases, R³ is CH₃, CH₂CH₃, CH₂CH₂CH₃, or CH(CH₃)₂. In some cases, the C₁₋₆ alkyl, C₂₋₄ alkenyl, and C₂₋₄ alkynyl is substituted with 1-3 substituents. In some cases, each of the 1-3 substituents independently is C₁₋₃ haloalkyl, C₀₋₆ alkylene(OH), C₀₋₆ alkylene-C₁₋₃ alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 3-7 total ring atoms and 1-3 heteroatoms selected from N, O, and S, or phenyl. In some cases, each of the 1, 2, or 3 substituents independently is from CH₃, CF₃, CF₂H, CFH₂, OH, OCH₃, OCF₃, CH₂OH, CH₂OCH₃, cyclopropyl, cyclobutyl, or phenyl. In some cases, R⁵ is CH₃, CH₂CH₃, CH₂CH₂CH₃, CH(CH₃)₂,

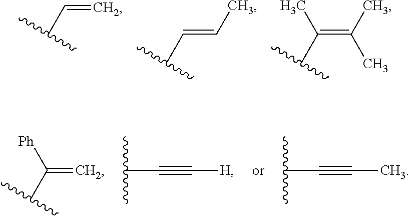

In some cases, R⁵ is cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing is optionally substituted with 1, 2, or 3 substituents, wherein each of the 1, 2, or 3 substituents independently is halo, C₁₋₃ alkyl, C₁₋₃ haloalkyl, C₀₋₆ alkylene(OH), or C₀₋₆ alkylene-C₁₋₃ alkoxy. In some cases, the cycloalkyl is cyclopropyl, cyclobutyl, cyclopentyl, or cyclohexyl, wherein each of the foregoing is optionally substituted with 1-3 substituents. In some cases, the cycloalkenyl is cyclopentenyl or cyclohexenyl, wherein each of the foregoing is optionally substituted with 1-3 substituents. In some cases, the heterocycloalkyl is aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiophenyl, oxazolidinyl, oxathiolidinyl, isoxazolidinyl, thiazohdinyl, isothiazolidinyl, piperidinyl, piperazinyl, tetrahydropyranyl, dioxanyl, tetrahydrothiopyranyl, dithianyl, morpholinyl, or thiomorpholinyl, wherein each of the foregoing is optionally substituted with 1-3 substituents. In some cases, the heterocycloalkenyl is dihydropyrrolyl, dihydrofuranyl, dihydrothiophenyl, dihydroisoxazolyl, tetrahydropyridinyl, dihydropyranyl, or dihydrothiopyranyl, wherein each of the foregoing is optionally substituted with 1-3 substituents. In some cases, R⁵ is CH₃, CF₃, CF₂H, CFH₂, CH₂CH₃, CH₂CH₂CH₃, CH(CH₃)₂,

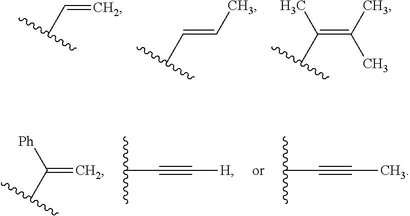

In some cases, R⁵ is cyclopropyl, cyclobutyl, cyclopentanyl, oxetanyl, or tetrahydrofuranyl. In some cases, R⁵ is CH₃, CH₂CH₃, CH₂CH₂CH₃, CH(CH₃)₂,

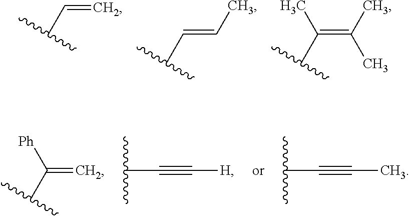

In some cases, R⁵ is CH₃, CH₂CH₃, CH₂CH₂CH₃, or CH(CH₃)₂. In some cases, R³ is CH₃, CH₂CH₃, CH₂CH₂CH₃, CH(CH₃)₂, CH═CH₂, CH═CHCH₃,

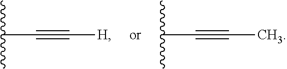

wherein each of the foregoing is optionally substituted with 1-3 substituents, and each substituent independently is C₁₋₃ haloalkyl, C₀₋₆ alkylene-OH, C₀₋₆ alkylene-C₁₋₃ alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl. In some cases, each substituent on R⁵ independently is CH₃, CF₃, CF₂H, CFH₂, OH, OCH₃, OCF₃, CH₂OH, CH₂OCH₃, cyclopropyl, cyclobutyl, or phenyl. In some cases, R⁵ is Br, Cl, F, OCH₂, SCH₃, CH₃, CH₂CH₃, CH₂CH₂CH₃, CH(CH₃)₂,

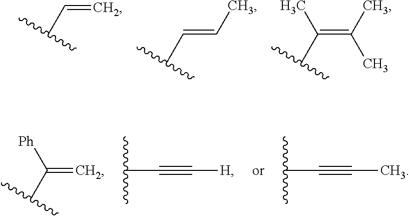

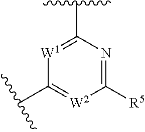

is

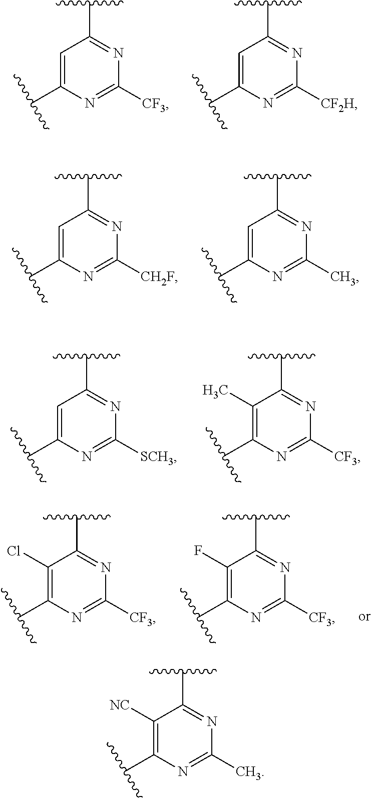

In some cases,

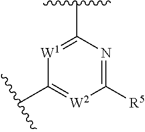

is

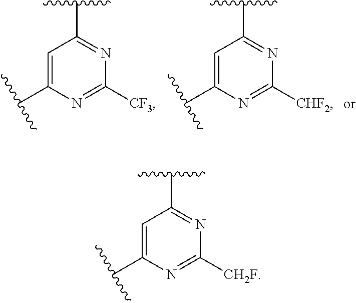

In some cases,

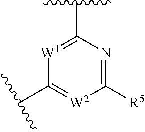

is

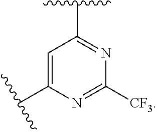 [SMILES: *c1cc(*)nc(C(F)(F)F)n1]

In some cases

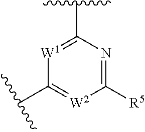

is

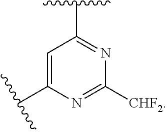 [SMILES: *c1cc(*)nc(C(F)F)n1]

In some cases, X is

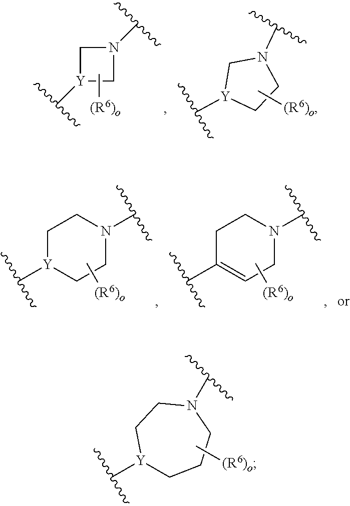

Y is N, C—H, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy; o is 0, 1, 2, 3, or 4; and each R⁶ independently is halo, CN, C₁₋₃ alkyl, C₂₋₃ alkenyl, C₁₋₃ haloalkyl, C₀₋₃ alkylene-OH, C₀₋₃ alkylene-C₁₋₃ alkoxy, deuterated C₀₋₃ alkylene-C₁₋₃ alkoxy, C₁₋₄ alkylene-N(R^{N1})₂, oxo, ═CH₂, spiro-cycloalkyl having 3-7 total atoms, spiro-cycloalkenyl having 4-7 total atoms, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 4-7 total ring atoms and 1-3 heteroatoms selected from N, O, and S, or a fused heterocycloalkenyl ring having 4-7 total ring atoms and 1 or 2 heteroatoms selected from N, O, and S; or two non-adjacent R⁶ join together to form a C₁₋₃ alkylene bridge, a C₂₋₃ alkenylene bridge, a C₁₋₃ ether bridge, or a C₁₋₃ thioether; or Y and an adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1-3 heteroatoms selected from N, O, and S, or a fused heterocycloalkenyl ring having 4-7 total ring atoms and 1 or 2 heteroatoms selected from N, O, and S; wherein the cycloalkyl, cycloalkenyl, heterocycloalkyl or heterocycloalkenyl of any of the foregoing is optionally substituted with 1 or more substituents; and each R^{N1} independently is H or C₁₋₄ alkyl. In some cases. X is

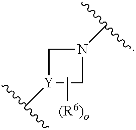

In some cases, X is

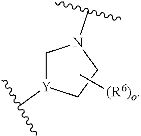

In some cases, X is

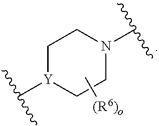

In some cases, X is

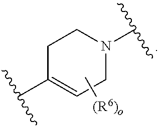

In some cases, X is

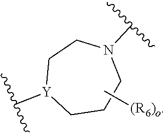

In some cases, Y is N. In some cases, Y is C—H. In some cases, Y is C-halo, C—CN, C—C₁₋₃ alkyl, C—C₁₋₃ haloalkyl, C—C₀₋₃ alkyleneOH, or C—C₀₋₃ alkylene-C₁₋₄ alkoxy. In some cases, Y is C—F, C—Cl, or C—CN. In some cases, Y is C—C₁₋₃ alkyl, C—C₁₋₃ haloalkyl. In some cases, Y is C—CH, C—CH₂CH₃, C—CH₂F, C—CHF₂, or C—CF₃. In some cases, Y is C—C₀₋₃ alkyleneOH or C—C₀₋₃ alkylene-C₁₋₄ alkoxy. In some cases, Y is C—OH, C—CH₂OH, C—OCH₃, or C—CH₂OCH₃. In some cases, o is 0. In some cases, o is 1. In some cases, o is 2. In some cases, o is 3. In some cases, o is 4. In some cases, at least one R⁶ is halo or CN. In some cases, at least one R⁶ is Br, Cl, F. or CN. In some cases, at least one R⁶ is oxo or ═CH₂. In some cases, at least one R⁶ is oxo. In some cases, o is 1 or 2 and each R⁶ independently is F. In some cases, at least one R⁶ is C₁₋₃ alkyl or C₁₋₃ haloalkyl. In some cases, at least one R⁶ is CH₃, CH₂F, CHF₂, or CF₃. In some cases, o is 1 or 2 and each R⁶ independently is CH₃. In some cases, at least one R⁶ is C₀₋₃ alkyleneOH, C₀₋₃ alkylene-C₁₋₃ alkoxy, deuterated C₀₋₃ alkylene-C₁₋₃ alkoxy, or C₁₋₄ alkylene-N(R^{N1}), and each R^{N1} independently is H or CH₃. In some cases, each R^{N1} independently is H. In some cases, at least one R⁶ is OH, CH₂OH, CH₂CH₂OH, OCH₃, OCD₃, or CH₂OCH₃, or CH, CH₂OCH₃. In some cases, at least one R⁶ is CH₂N(CH₃)₂, CH₂NH(CH₃), or CH₂NH₂. In some cases, at least one R⁶ is OH, CH₂OH, OCH₃, OCD₃, CH₂OCH₃, or CH₂N(CH₃)₂. In some cases, o is 1 and R⁶ is OH, CH—OH, OCH₃, or CH₂OCH₃. In some cases, at least one R⁶ is spiro-cycloalkyl having 3-7 total atoms, spiro-cycloalkenyl having 4-7 total atoms, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S, wherein any of the foregoing is optionally substituted with 1 or more substituents. In some cases, at least one R⁶ is spiro-cycloalkyl having 3-7 total ring atoms or spiro-heterocycloalkyl having 4-7 total ring atoms and 1 or 2 heteroatoms selected from N, O and S, wherein the cycloalkyl and heterocycloalkyl are optionally substituted with 1 or more substituents. In some cases, at least one R⁶ is spiro-cyclopropyl, spiro-cyclobutyl, spiro-oxetanyl, or spiro-tetrahydrofuranyl, wherein any of the foregoing is optionally substituted with 1 or more substituents. In some cases, o is 1 and R⁶ is spiro-cyclopropyl, wherein the cyclopropyl is optionally substituted with 1 or more substituents. In some cases, two adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 4-7 total ring atoms and 1-3 heteroatoms selected from N, O, and S, or a fused heterocycloalkenyl having 4-7 total ring atoms and 1 or 2 heteroatoms selected from N, O, and S; or Y and an adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1-3 heteroatoms selected from N, O, and S, or a fused heterocycloalkenyl ring having 4-7 total ring atoms and 1 or 2 heteroatoms selected from N, O, and S; wherein the cycloalkyl, cycloalkenyl, heterocycloalkyl, or heterocycloalkenyl of any of the foregoing is optionally substituted with 1 or more substituents. In some cases, two adjacent R⁶, together with the atoms to which they are attached, form a fused cycloalkyl ring having 3-7 total ring atoms, wherein the fused cycloalkyl ring is optionally substituted with 1 or more substituents. In some cases, Y and an adjacent R⁶, together with the atoms to which they are attached, form a fused cycloalkyl ring having 3-7 total ring atoms, wherein the fused cycloalkyl ring is optionally substituted with 1 or more substituents. In some cases, the fused cycloalkyl ring of any of the foregoing is fused-cyclopropyl, fused-cyclobutyl, or fused-cyclopentyl, wherein any of the foregoing is optionally substituted with 1 or more substituents. In some cases, the spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is unsubstituted. In some cases, the spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 1-4 substituents. In some cases, the spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 1 or 2 substituents. In some cases, each substituent independently is halo, C₁₋₃ alkyl, C₁₋₃ haloalkyl, C₀₋₂ alkyleneOH, C₀₋₂ alkyleneC₁₋₃ alkoxy, or C₀₋₂ alkyleneCN. In some cases, each substituent independently is halo, OH, C₁₋₃ alkoxy, or CN. In some cases, each substituent independently is F, Cl, OH, OCH₃, OCH₂CH₃, or CN. In some cases, two non-adjacent R⁶ join together to form a C₁₋₃ alkylene bridge, a C₂₋₃ alkenylene bridge, a C₁₋₃ ether bridge, or a C₁₋₃ thioether bridge. In some cases, two non-adjacent R⁶ join together to form a C₁₋₃ alkylene bridge, a C₂₋₃ alkenylene bridge, or a C₁₋₃ ether bridge. In some cases, two non-adjacent R⁶ join together to form a C₁₋₃ alkylene bridge or a C₂₋₃ alkenylene bridge. In some cases, two non-adjacent R⁶ join together to form a C₁₋₃ ether bridge or a C₁₋₃ thioether bridge. In some cases, two non-adjacent R⁶ join together to form a C₁₋₂ alkylene bridge or a C₁₋₃ ether bridge. In some cases, two non-adjacent R⁶ join together to form a C₁ alkylene bridge

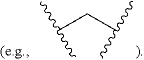 [SMILES: *C*]

In some cases, two non-adjacent R⁶ join together to form a C₂ alkylene bridge

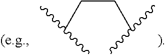 [SMILES: *CC*]

In some cases, two non-adjacent R⁶ join together to form a C₃ alkylene bridge

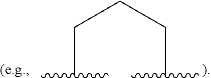 [SMILES: *CCC*]

In some cases, two non-adjacent R⁶ join together to form a C₂ alkenylene bridge

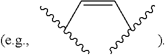 [SMILES: */C=C\*]

In some cases, two non-adjacent R⁶ join together to form a C₁₋₃ alkenylene bridge

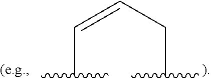 [SMILES: */C=C\C*]

In some cases, two non-adjacent R⁶ join together to form a C₁₋₃ ether bridge

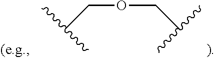 [SMILES: *COC*]

In some cases, two non-adjacent R⁶ join together to form a C₁₋₃ thioether bridge

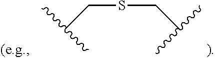 [SMILES: *CSC*]

In some cases, two non-adjacent R⁶ join together to form —CH₂—, —CH₂CH₂—, —CH₂CH₂CH₂—, —CH₂—CH═CH— or —CH₂OCH₂—. In some cases, two non-adjacent R⁶ join together to form

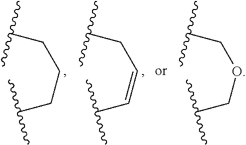

In some cases, X is

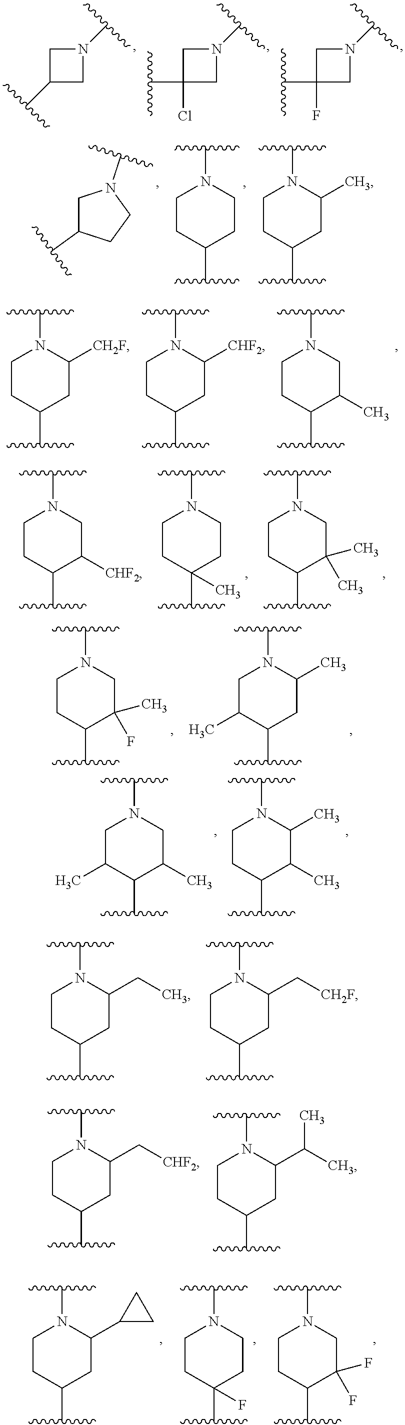

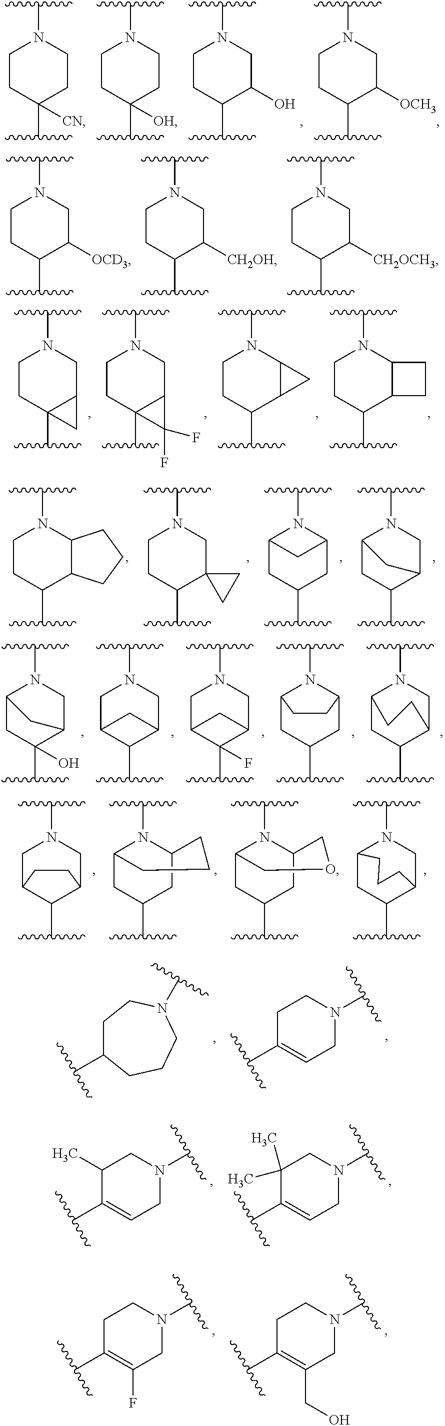

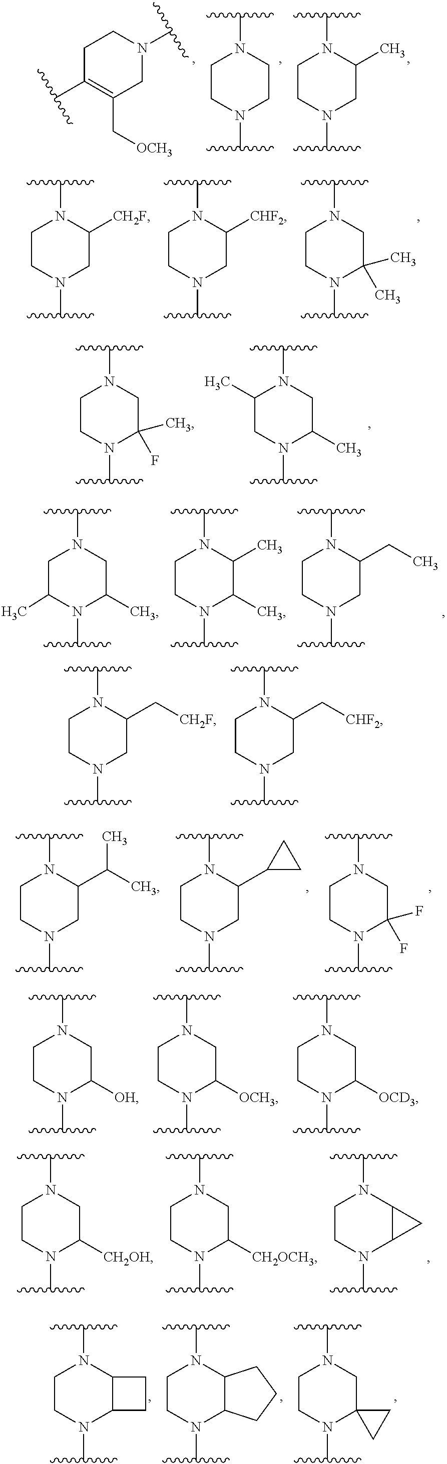

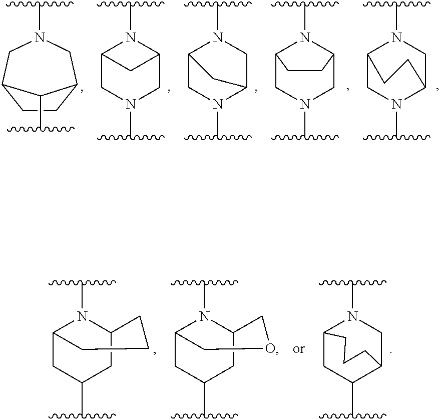

In some cases X is

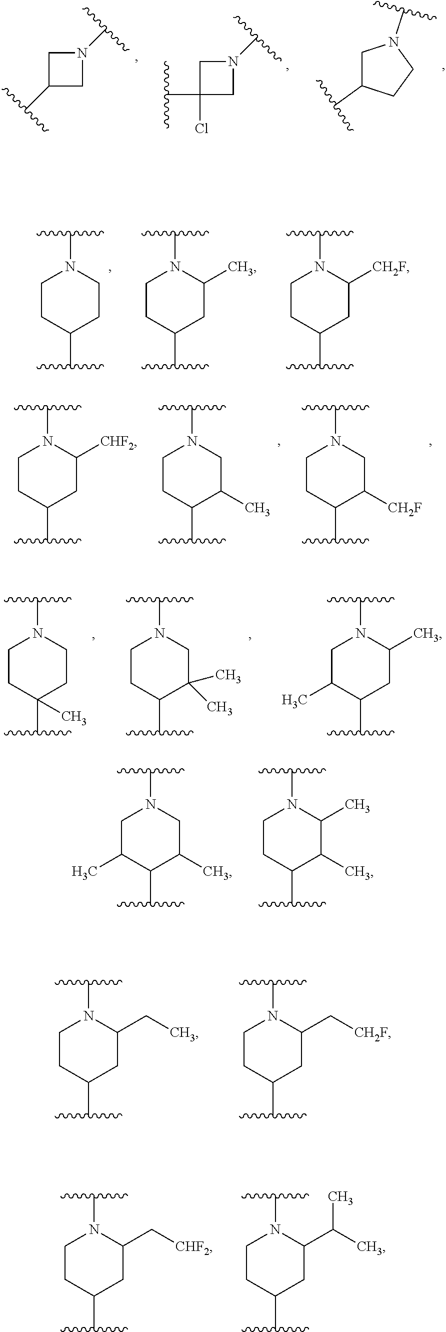

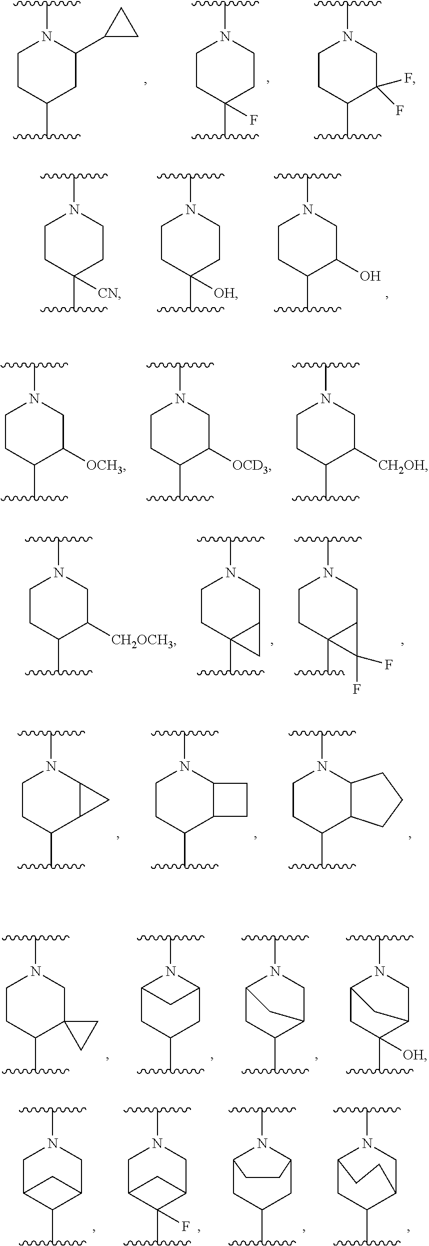

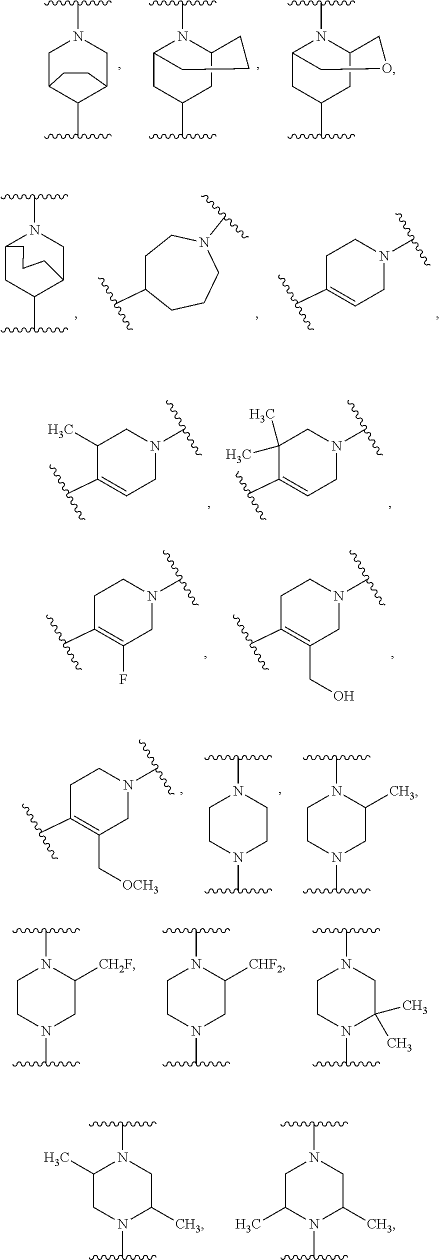

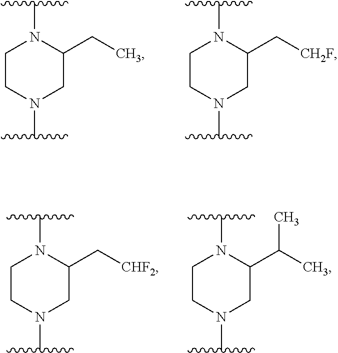

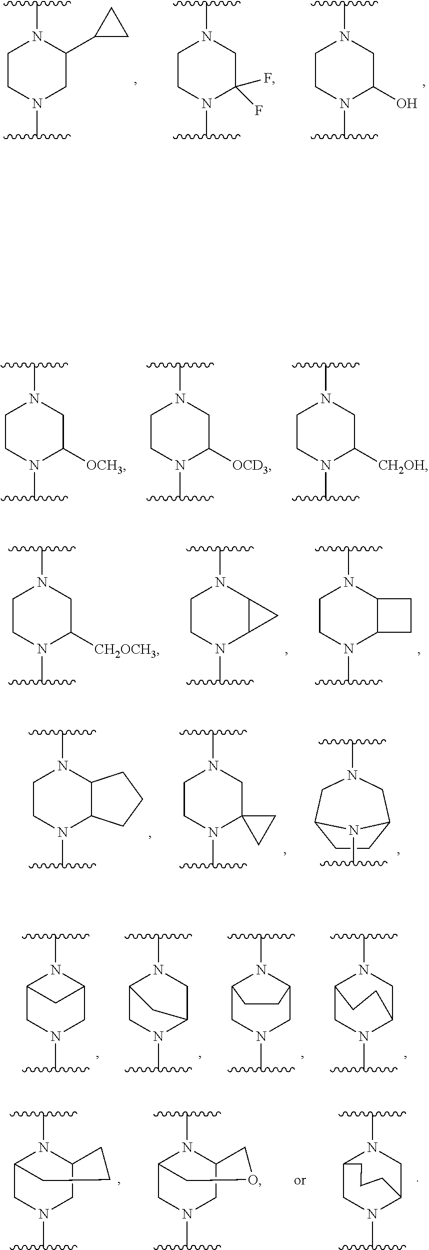

In some cases, X is

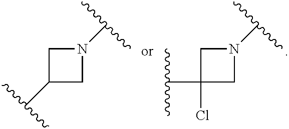 [SMILES: *C1CN(*)C1 ; *N1CC(*)(Cl)C1]

In some cases, X is

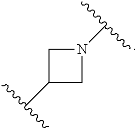 [SMILES: *C1CN(*)C1]

In some cases, X

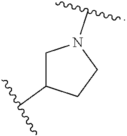 [SMILES: *C1CCN(*)C1]

In some cases, X is

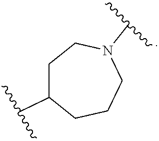 [SMILES: *C1CCCN(*)CC1]

In some cases, X is

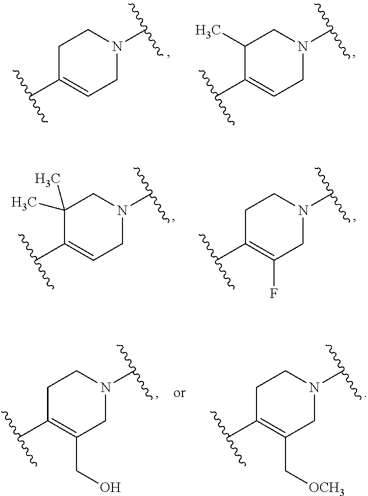

In some cases, X is

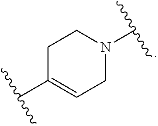 [SMILES: *C1=CCN(*)CC1]

In some cases, X is

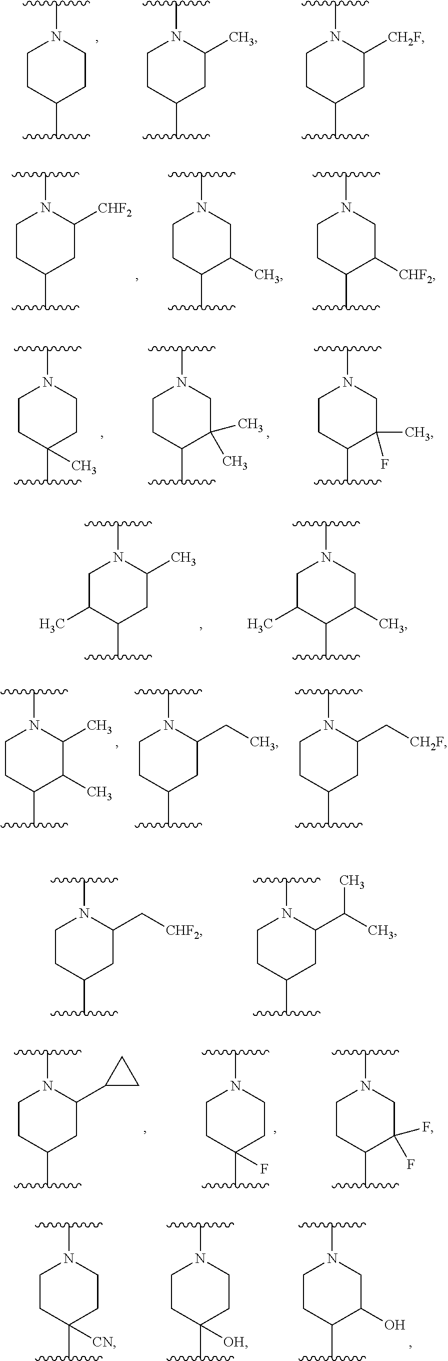

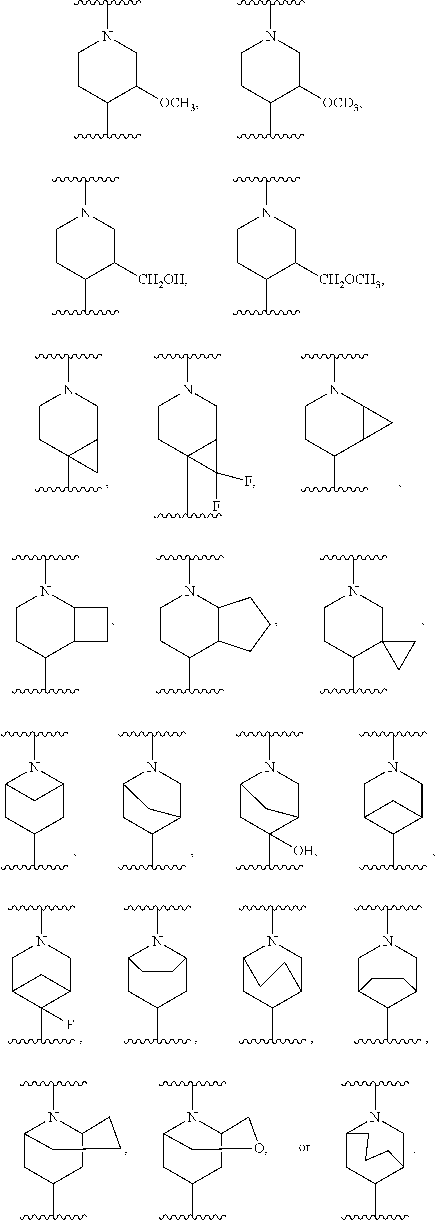

In some cases, X is

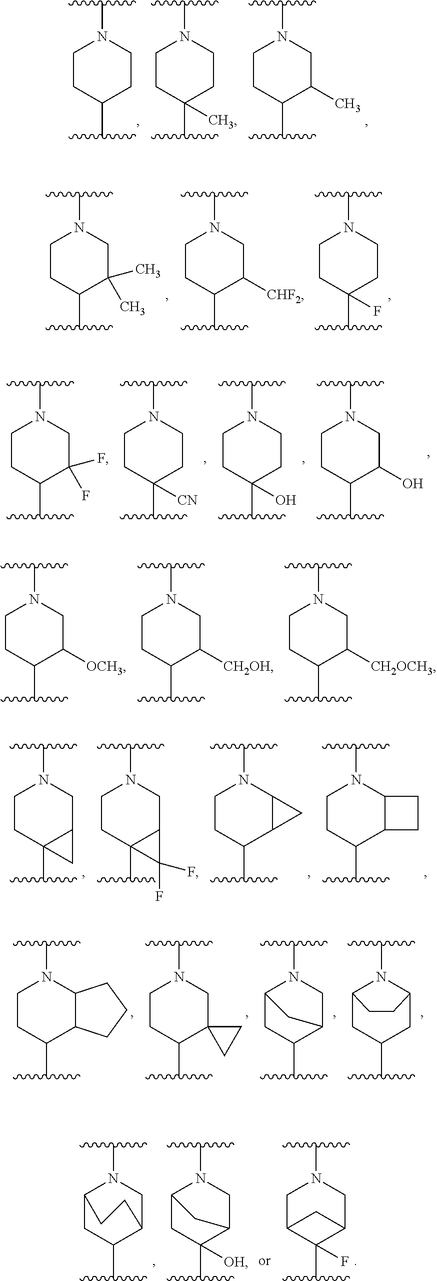

In some cases, X is

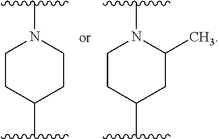

In some cases, X is

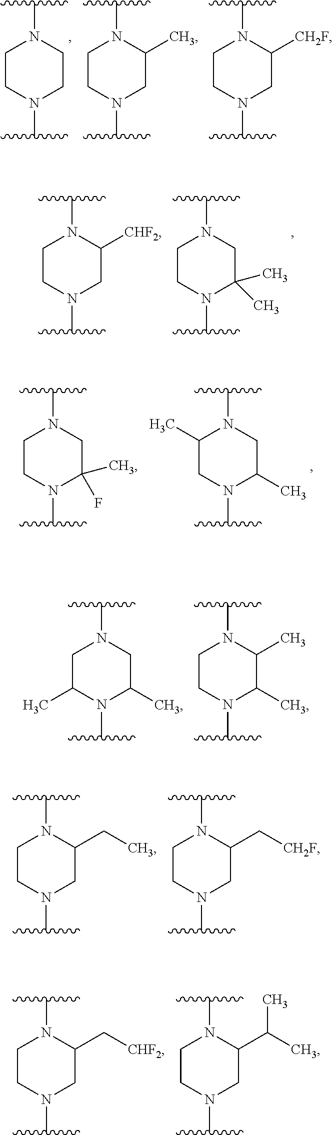

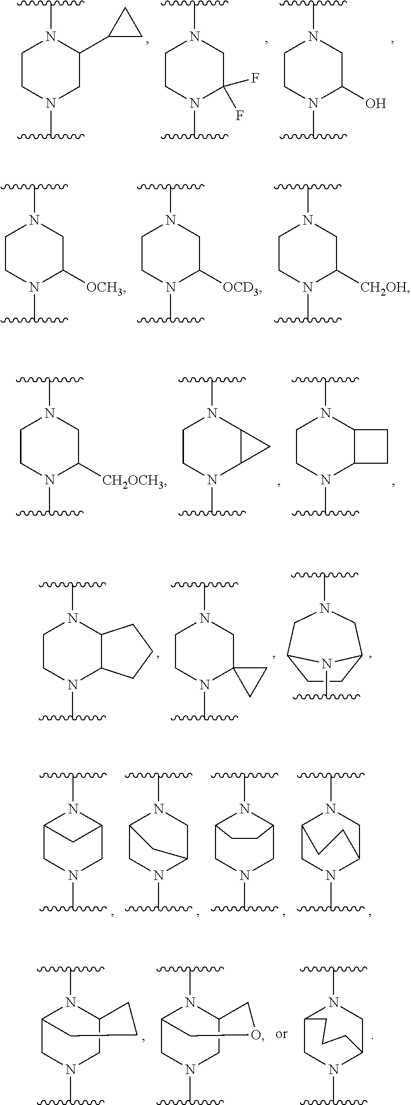

In some cases, X is

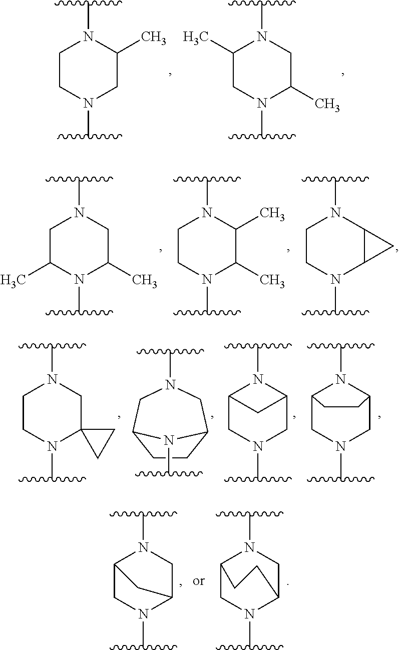

In some cases, X is

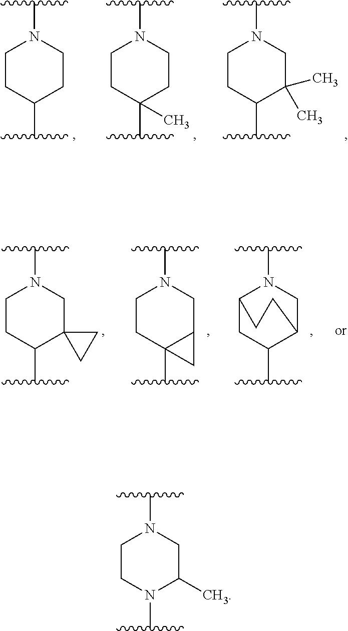

In some cases, X is

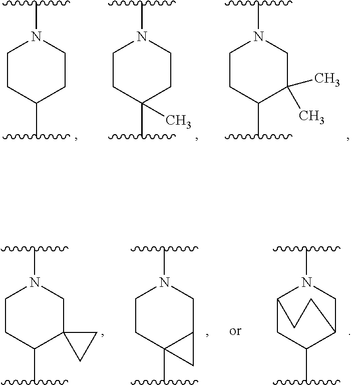

In some cases, X is

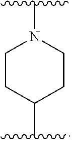 [SMILES: *C1CCN(*)CC1]

In some cases, X is

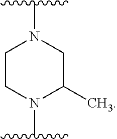 [SMILES: *N1CCN(*)C(C)C1]

In some cases, X is

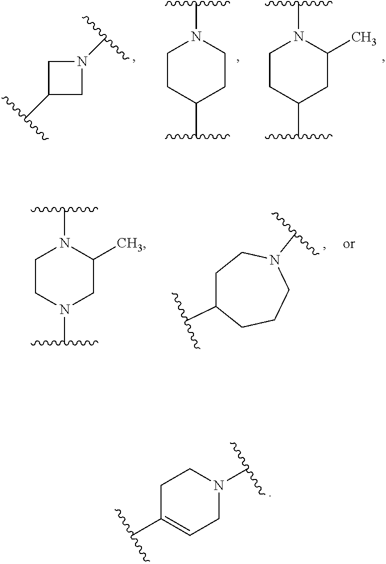

In some cases, X is

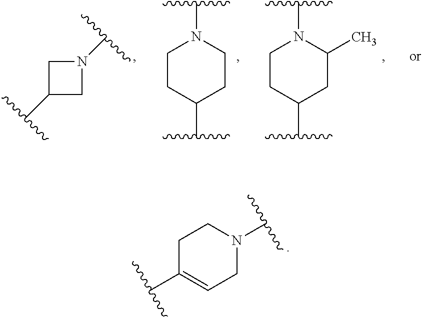

In some cases, Z is phenyl optionally substituted with 1-4 substituents. In some cases, each substituent independently is halo, C₀₋₃ alkyleneCN, C₀₋₃ alkyleneOH, C₀₋₃ alkylene-C₁₋₄ alkoxy, C₀₋₃ alkylene-C₁₋₄ thioalkoxy, or

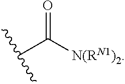

In some cases, each R^{N1} independently is H or CH₃. In some cases, each R^{N3} independently is H. In some cases, each of the 1-4 substituents independently is F, Cl, CN, OCH₃, SCH₃, CH₂OH, or

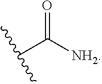 [SMILES: *C(N)=O]

In some cases, Z is

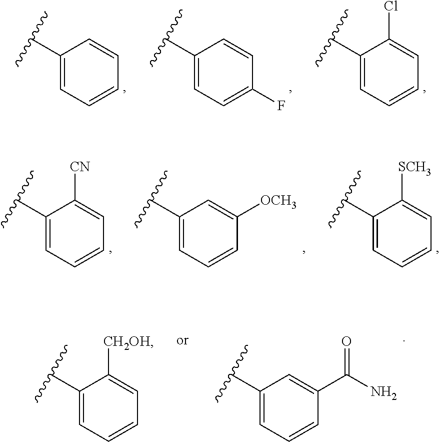

In some cases, Z is

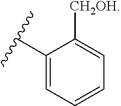 [SMILES: *c1ccccc1CO]

In some cases, Z is heteroaryl comprising 5 or 6 total ring atoms and 1-3 heteroatoms selected from N, O, and S, wherein the heteroaryl is optionally substituted with 1 or more substituents. In some cases, the heteroaryl comprises 5 total ring atoms. In some cases, the heteroaryl comprises 6 total ring atoms, in some cases, the heteroaryl is pyrrolyl, furanyl, thiophenyl, pyrazolyl, imidazolyl, thiazolyl, isothiazolyl, oxazolyl, isoxazolyl, triazolyl, thiadiazoyl, oxadiazol, pyridyl, pyridazinyl, pyrimidinyl, pyrazinyl, or triazinyl, wherein each of the foregoing is optionally substituted with 1 or more substituents. In some cases, the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, or triazolyl, wherein each of the foregoing is optionally substituted with 1 or more substituents. In some cases, the heteroaryl is pyrazolyl, imidazolyl, thiazolyl, isothiazolyl, oxazolyl, isoxazolyl, or triazolyl, wherein each of the foregoing is optionally substituted with 1 or more substituents. In some cases, the heteroaryl is pyrazolyl, imidazolyl, thiazolyl, or isothiazolyl, wherein each of the foregoing is optionally substituted with 1 or more substituents. In some cases, the heteroaryl is pyrazolyl, wherein the pyrazolyl is optionally substituted with 1 or more substituents. In some cases, the heteroaryl is imidazolyl, wherein the imidazolyl optionally substituted with 1 or more substituents. In some cases, the heteroaryl is thiazolyl, wherein the thiazolyl is optionally substituted with 1 or more substituents. In some cases, the heteroaryl is isothiazolyl, wherein the isothiazolyl is optionally substituted with 1 or more substituents. In some cases, the heteroaryl is pyridyl, pyridazinyl, pyrimidinyl, or pyrazinyl, wherein each of the foregoing is optionally substituted with 1 or more substituents. In some cases, the heteroaryl pyridyl, wherein the pyridyl is optionally substituted with 1 or more substituents. In some cases, the heteroaryl is pyrazolyl, thiazolyl, pyridyl, or pyridazinyl. In some cases, the heteroaryl is pyrazolyl or pyridyl, wherein each of the foregoing is optionally substituted with 1 or more substituents.

In some cases, the heteroaryl is unsubstituted. In some cases, the heteroaryl is substituted with 1-4 substituents. In some cases, the heteroaryl is substituted with 1 or 2 substituents. In some cases, the heteroaryl is substituted with 3 or 4 substituents. In some cases, the heteroaryl is substituted with 1 substituent. In some cases, the heteroaryl is substituted with 2 substituents. In some cases, the heteroaryl is substituted with 3 substituents. In some cases, the heteroaryl is substituted with 4 substituents. In some cases, each of the 1-4 substituents independently is halo CN, C₁₋₆ alkyl, C₁₋₆ haloalkyl, C₂₋₆ alkenyl, C₂₋₆ haloalkenyl, C₀₋₆ alkylene-OH, C₀₋₆ alkylene-C₁₋₃ alkoxy, C₁₋₆ alkylene-N(R^{N1})₂ wherein each R^{N1} independently is H or C₂₋₃ alkyl, C₀₋₃ alkylene-cycloalkyl having 3-6 total ring atoms, C₀₋₂ alkylene-heterocycloalkyl having 3-6 total ring atoms and 1-3 heteroatoms selected from N, O, and S, or C₀₋₂ alkylene-phenyl, wherein the alkyl, alkenyl, C₀₋₆ alkylene-C₁₋₃ alkoxy, cycloalkyl, heterocycloalkyl, and phenyl substituents are each independently substituted with 1 or more further substituents. In some cases, the alkyl, alkenyl, C₀₋₆ alkylene-C₁₋₃ alkoxy, cycloalkyl, heterocycloalkyl, and phenyl substituents are each independently substituted with 1-3 further substituents. In some cases, the alkyl, alkenyl, C₀₋₆ alkylene-C₁₋₃ alkoxy, cycloalkyl, heterocycloalkyl, and phenyl substituents are each independently substituted with 1 or 2 further substituents. In some cases, the alkyl, alkenyl, C₀₋₃ alkylene-C₁₋₃ alkoxy, cycloalkyl, heterocycloalkyl, and phenyl substituents are each independently substituted with 1 further substituent.

In some cases, each further substituent independently is D, halo, C₁₋₃ alkyl, C₁₋₃ haloalkyl, C₁₋₂ alkyleneOH, C₁₋₂ alkylene-C₁₋₃ alkoxy, C₁₋₃ deuterated alkoxy. N(R^{N1})₂, (C═O)C₁₋₃ alkyl, cycloalkyl having 3-5 total ring atoms, heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S, or two adjacent further substituents, together with the atoms to which they are attached, form fused-cycloalkyl having 3-5 total ring atoms, or fused-heterocycloalkyl having 3-5 total ring atoms and 1 or 2 heteroatoms selected from N, O, and S, wherein each of the foregoing cycloalkyl and heterocycloalkyl groups independently is optionally substituted with 1 or 2 substituents, and each substituent independently is halo or C₁₋₃ alkyl, and each R^{N1} independently is H or C₁₋₃ alkyl. In some cases, each further substituent independently is D, halo, OH, CH₃, OCH₃, or OCD₃. In some cases, each further substituent independently is D, Br, Cl, F, OH, CH₃, OCH₃, or OCD₃. In some cases, each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, azetidinyl, spiro-oxetanyl or spiro-azetidinyl; wherein each of the foregoing oxetanyl, azetidinyl, spiro-oxetanyl, and spiro-azetidinyl is optionally substituted with F, CH₃, or a combination thereof. In some cases, each further substituent independently is D, Br, Cl, F, OH, CH₃, CH(CH₃)₂, CF₃, CF₂H, CFH₂, OCH₃, OCD₃, CH₂OCH₃, N(CH₃)₂, (C═O)CH₃,

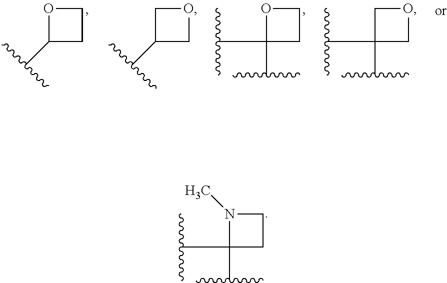

In some cases, each further substituent independently is D, CH₃, OCH₃, OCD₃, N(CH₃)₂,

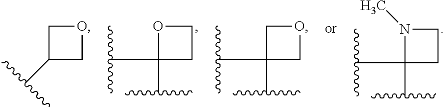

In some cases, the heteroaryl is substituted with CN. In some cases, the heteroaryl is substituted with halo. In some cases, the heteroaryl is substituted with Br, Cl, F, or CN. In some cases, the heteroaryl is substituted with C₁₋₆ alkyl, wherein the alkyl is optionally substituted with 1 or more further substituents. In some cases, the heteroaryl is substituted with CH₃, CH₂CH₃, CH₂CH₂CH₃, or CH(CH₃)₂, wherein each of the foregoing independently is optionally substituted with 1 or more further substituents. In some cases, heteroaryl is substituted with CH, that is optionally substituted with 1 or more further substituents. In some cases, the C₁₋₆ alkyl is unsubstituted. In some cases, the C₁₋₆ alkyl is CH₃, CH₂CH₃, CH₂CH₂CH₃, or CH(CH₃)₂. In some cases, the C₁₋₆ alkyl is substituted with 1-3 substituents, and each of the 1-3 substituents independently is deuterium and halo. In some cases, the substituted C₁₋₆ alkyl is CD₃. In some cases, the heteroaryl is substituted with C₁₋₆ haloalkyl. In some cases, the C₁₋₆ haloalkyl is CF₃, CHF₂, CH₂F, CH₂CHF₂, CH₂CH₂F, CH(CH₂F)₂, CH(CH₃)CH₂F, or CH(CH₃)CHF₂. In some cases, the heteroaryl is substituted with C₂₋₆ alkenyl, wherein the alkenyl is optionally substituted with 1 or more further substituents. In some cases, the C₂₋₆ alkenyl is CH═CH₂, CH₂CH═CH₂, or CH═CHCH₃, and each of the foregoing independently is optionally substituted with 1 or more further substituents. In some cases, the C₂₋₆ alkenyl is unsubstituted. In some cases, the C₂₋₆ alkenyl is CH═CH₂, CH₂CH═CH₂, or CH═CHCH₃. In some cases, the C₂₋₆ alkenyl is substituted with 1-3 substituents, and each of the 1-3 substituents independently is deuterium, halo, OH, OCH₃, and OCD₃. In some cases, the heteroaryl is substituted with C₂₋₆ haloalkenyl. In some cases, the C₂₋₆ haloalkenyl is C(═CH₂)CH₂F. In some cases, the heteroaryl is substituted with C₀₋₆ alkylene-OH. In some cases, the C₀₋₆ alkylene-OH is OH, CH₂OH, or CH₂CH₂OH. In some cases, C₀₋₆ alkylene-OH is OH, CH₂OH, CH₂CH₂OH, CH(CH)CH₂OH, C(CH₃)₂OH, C(CH₃)₂CH₂OH, or CH₂C(CH₃)₂OH. In some cases, the heteroaryl is substituted with C₀₋₆ alkylene-C₁₋₃ alkoxy, wherein the alkoxy is optionally substituted with 1 or more further substituents. In some cases, the C₀₋₆ alkylene-C₁₋₃ alkoxy is OCH₃, CH₂OCH₃, CH₂CH₂OCH₃, CH₂CH₂OCH₂CH₃, CH₂CH₂CH₂OCH₃, CH(CH₃)OCH₃, CH(CH₃)CH₂OCH₃, CH(OCH₃)CH₂OCH₃, CH(CH₃)(OCH₃)CH₂OCH₃, C(CH₃)₂OCH₃, C(CH₃)₂CH₂OCH₃, CH₂CH(CH₃)OCH₃, CH₂(CH₂)(OCH₃)OCH₃, CH₂C(CH₃)₂OCH₃, or CH₂C(CH₃)₂OCH₃, and each of the foregoing independently is optionally substituted with 1 or more further substituents. In some cases, the C₀₋₆ alkylene-C₁₋₃ alkoxy, is CH(CH₃)OCH₃ or CH₂CH₂OCH₃, and each of the foregoing independently is optionally substituted with 1 or more further substituents. In some cases, the heteroaryl is substituted with OCH₃, OCD₃, CH₂OCH₃, CH₂OCD₃, CH₂CH₂OCH₃, CH₂CH₂OCD₃, CHFCH₂OCH₃, CF₂CH₂OCH₃, CH₂CH₂OCH₃, CH₂CH₂CH₂OCD₃, CH(CH₃)CH₂OCH₃, C(CH₃)₂CH₂OCH₃, CH₂CH(CH₃)OCH₃, CH₂C(CH₃)₂OCH₃, CH(CH₃)CH₂OCD₃, C(CH₃)₂CH₂OCD₃, CH₂CH(CH₃)OCD₃, CH₂C(CH₃)₂OCD₃, or a combination thereof. In some cases, the heteroaryl is substituted with C₀₋₆ alkylene-N(R^{N1})₂. In some cases, C₀₋₆ alkylene-N(R^{N1})₂ is NH₂, CH₂NH₂, CH₂NHCH₃, CH₂N(CH₃)₂, CH₂CH₂NH₂, CH₂CH₂NHCH₃, or CH₂CH₂N(CH₃)₂. In some cases, the heteroaryl is substituted with C₀₋₂ alkylene-C₃₋₆ cycloalkyl, wherein the cycloalkyl is optionally substituted with 1 or more further substituents. In some cases, the cycloalkyl of the C₀₋₂ alkylene-cycloalkyl is cyclopropyl, cyclobutyl, cyclopentyl, or cyclohexyl, and each of the foregoing independently is optionally substituted with 1 or more further substituents. In some cases, the cycloalkyl of the C₀₋₂ alkylene-cycloalkyl atoms is cyclopropyl or cyclobutyl, and each of the foregoing independently is optionally substituted with 1 or more further substituents. In some cases, the C₀₋₂ alkylene-cycloalkyl is unsubstituted. In some cases, the C₀₋₂ alkylene-cycloalkyl is substituted with 1-3 substituents. In some cases, each substituent independently is selected from halo, OH, CH₃, OCH₃, or OCD₃. In some cases, the C₀₋₂ alkylene-cycloalkyl is substituted with 1-3 substituents, and each substituent independently is Br, Cl, F, OH, CH₃, OCH₃, or OCD₃. In some cases, the optionally substituted C₀₋₂ alkylene-cycloalkyl is,

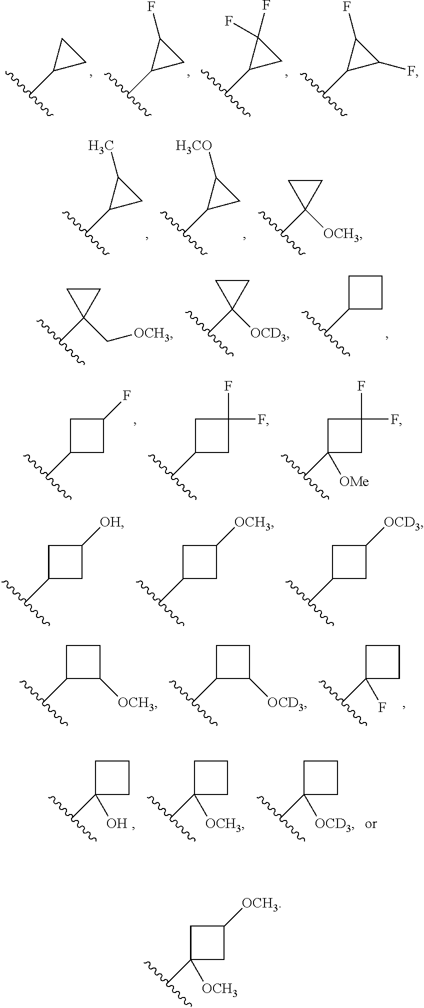

In some cases, the heteroaryl is substituted with C₀₋₂ alkylene-heterocycloalkyl having 3-6 total ring atoms and 1-3 heteroatoms selected from N, O, and S, wherein the heterocycloalkyl is optionally substituted with 1 or more further substituents. In some cases, the heterocycloalkyl of the C₀₋₂ alkylene-heterocycloalkyl is azetidinyl, pyrrolidinyl, piperdinyl, pyrazolidinyl, oxetanyl, tetrahydrofuranyl, tetrahydropyranyl, isoxazolidinyl, or morpholinyl, and each of the foregoing independently is optionally substituted with 1 or more further substituents. In some cases, the heterocycloalkyl of the C₀₋₂ alkylene-heterocycloalkyl is azetidinyl, oxetanyl, tetrahydrofuranyl, tetrahydropyranyl, or morpholinyl, and each of the foregoing is optionally substituted with 1 or more further substituents. In some cases, the heterocycloalkyl of the C₀₋₂ alkylene-heterocycloalkyl is azetidinyl or oxetanyl, and each of the foregoing is optionally substituted with 1 or more further substituents. In some cases, the heterocycloalkyl of the optionally substituted C₀₋₂ alkylene-heterocycloalkyl is azetidinyl, oxetanyl, pyrrolidinyl, pyrazolidinyl, tetrahydrofuranyl, tetrahydropyranyl, or piperidinyl. In some cases, the heterocycloalkyl of the C₀₋₂ alkylene-heterocycloalkyl is azetidinyl, wherein the azetidinyl is optionally substituted with 1 or more further substituents. In some cases, the heterocycloalkyl of the C₀₋₂ alkylene-heterocycloalkyl is oxetanyl, wherein the oxetanyl is optionally substituted with 1 or more further substituents. In some cases, the C₀₋₂ alkylene-phenyl is phenyl or CH₂-phenyl, wherein each of the foregoing independently is optionally substituted with 1 or more further substituents. In some cases, the C₀₋₂ alkylene-heterocycloalkyl is unsubstituted. In some cases, the C₀₋₂ alkylene-heterocycloalkyl is substituted with 1-3 substituents. In some cases, the C₀₋₂ alkylene-heterocycloalkyl is substituted with 1 or 2 substituents. In some cases, the C₀₋₂ alkylene-heterocycloalkyl is substituted with 2 substituents. In some cases, the C₀₋₂ alkylene-heterocycloalkyl is substituted with 1 substituent. In some cases, each substituent independently is halo, OH, CH₃, OCH₃, or OCD₃. In some cases, each substituent independently is Br, Cl, F, OH, CH₃, CF₃, CF₂H, CH₂F, OCH₃, OCD₃, or C(═O)CH₃, D, Br, Cl, F, OH, CH, CH(CH₃)₂, CF₃, CF₂H, CFH₂, OCH₃, OCD₃, CH₂OCH₃, N(CH₃)₂, (C═O)CH₃,

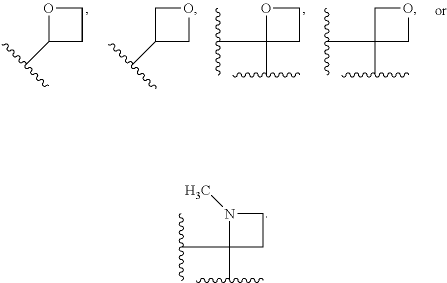

In some cases, the C₀₋₂ alkylene-heterocycloalkyl is

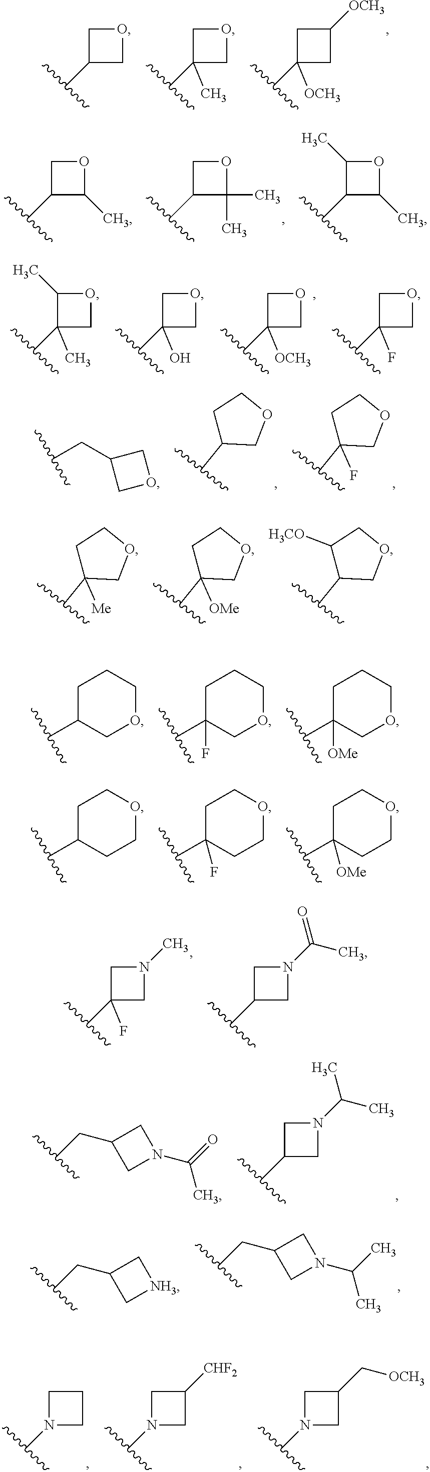

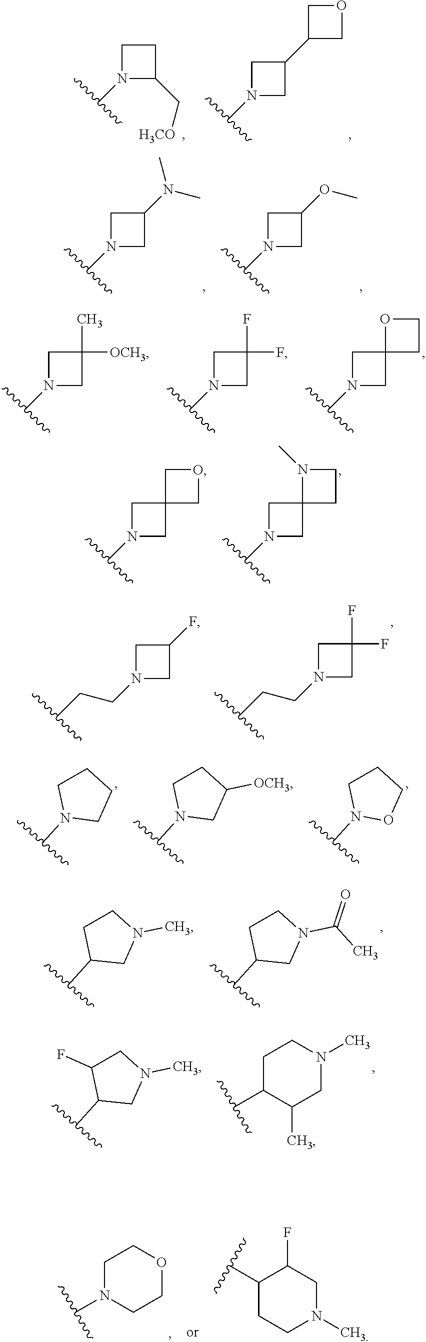

In some cases, each substituent of the heteroaryl independently is Br, Cl, F, CN, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH)₂OH, NH₂, CH₂NH₂, CH₂NHCH₃, CH₂N(CH₃)₂, CH₂CH₂NH₃, CH₂CH₂NHCH₃, CH₂CH₂N(CH₃)₂, C₁₋₃ alkyl selected from CH₃, CH₂CH₃, CH₂CH₂CH₃, and CH(CH₃)₂, C₂₋₆ alkenyl selected from CH═CH₂, CH₂CH═CH₂, and CH═CHCH₃, C₀₋₆ alkylene-C₁₋₃ alkoxy selected from OCH₃, CH₂OCH₃, CH₂CH₂OCH₃, CH₂CH₂OCH₂CH₂CH₂CH₂CH₂OCH₃, CH(CH)OCH₃, CH(CH₃)CH₂OCH₃, CH(OCH₃)CH₂OCH₃, CH(CH₃)(OCH₃)CH₂OCH₃, C(CH₁)₂OCH₃, C(CH₃)CH₂OCH₃, CH₂CH(CH₃)OCH₃, CH(CH₃)(OCH₃)OCH₃, CH₂C(CH₃)₂OCH₃, and CH₂C(CH₃)₂OCH₃, cycloalkyl selected from cyclopropyl, cyclobutyl, cyclopentyl, and cyclohexyl, or heterocycloalkyl selected from azetidinyl, pyrrolidinyl, piperidinyl, pyrazolidinyl, oxetanyl, tetrahydrofuranyl, tetrahydropyranyl, isoxazolidinyl, and morpholinyl; wherein each of the C₁₋₆ alkyl, C₂₋₆ alkenyl, C₀₋₆ alkylene-C₁₋₃ alkoxy, cycloalkyl, and heterocycloalkyl substituents independently is substituted with 1-3 further substituents and each further substituent independently is D, halo, C₁₋₃ alkyl, C₁₋₃ haloalkyl, C₁₋₂ alkyleneOH, C₁₋₂ alkylene-C₁₋₄ alkoxy, C₁₋₃ deuterated alkoxy, N(R^{N1})₂, (C═O)C₁₋₃ alkyl, cycloalkene having 3-5 total ring atoms, heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S, or two adjacent further substituents, together with the atoms to which they are attached, form fused-cycloalkyl having 3-5 total ring atoms or fused-heterocycloalkyl having 3-5 total ring atoms and 1 or 2 heteroatoms selected from N, O, and S. In some cases, each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, azetidinyl, spiro-oxetanyl or spiro-azetidinyl; wherein each of the foregoing oxetanyl, azetidinyl, spiro-oxetanyl, and spiro-azetidinyl is optionally substituted with F, CH₃, or a combination thereof. In some cases, each further substituent independently is D, Br, Cl, F, OH, CH₃, CF₃, CF₂H, CFH₂, OCH₃, CH₂OCH₃, OCD₃, N(CH₃)₂, (C═O)CH₃,

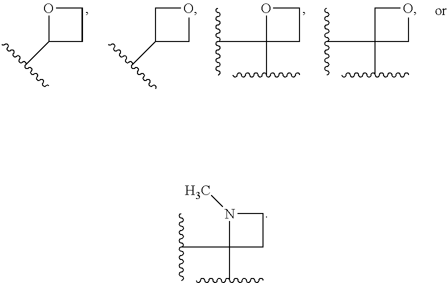

In some cases, each substituent of the heteroaryl of Z independently is Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃, CH₂CH₂CH₂OCH₃, CH₂CH₂CH₂OCD₃, CH(CH₃)OCH₃, CH(CH₃)CH₂OCH₃, CH(OCH₃)CH₂OCH₃, CH(CH)(OCH₃)CH₂OCH₃, CH(CH₂F)(CH₃)CH₂OCD₃, CH(CH₃)CH₂OCD₃, C(CH₃)₂OCH₃, C(CH₃)₂CH₂OCH₃, C(CH₃)₂CH₄OCD₃, CH₂CH(CH₃)OCH₃, CH₂(CH₃)(OCH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

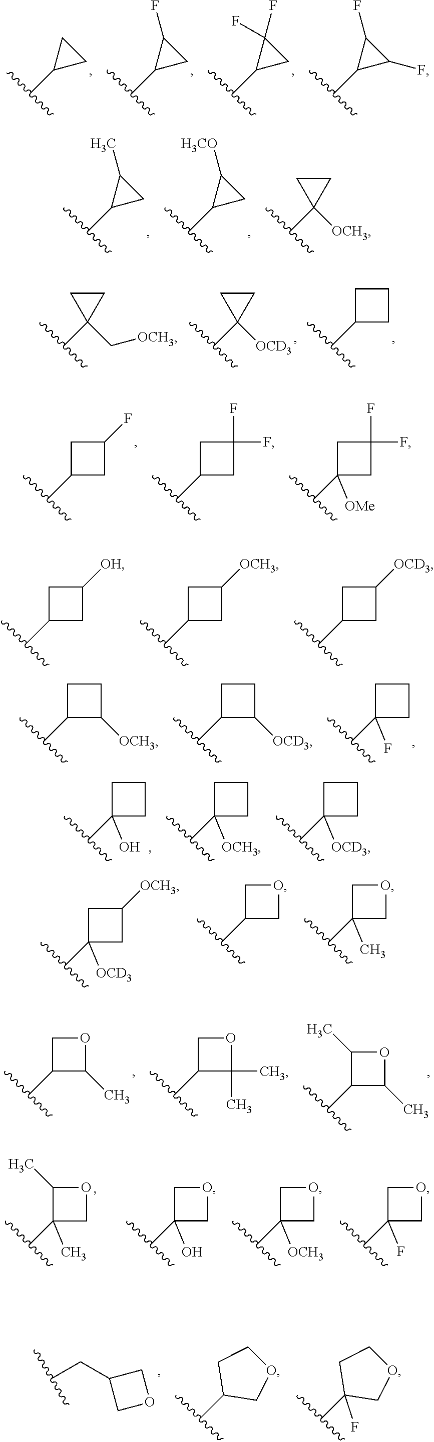

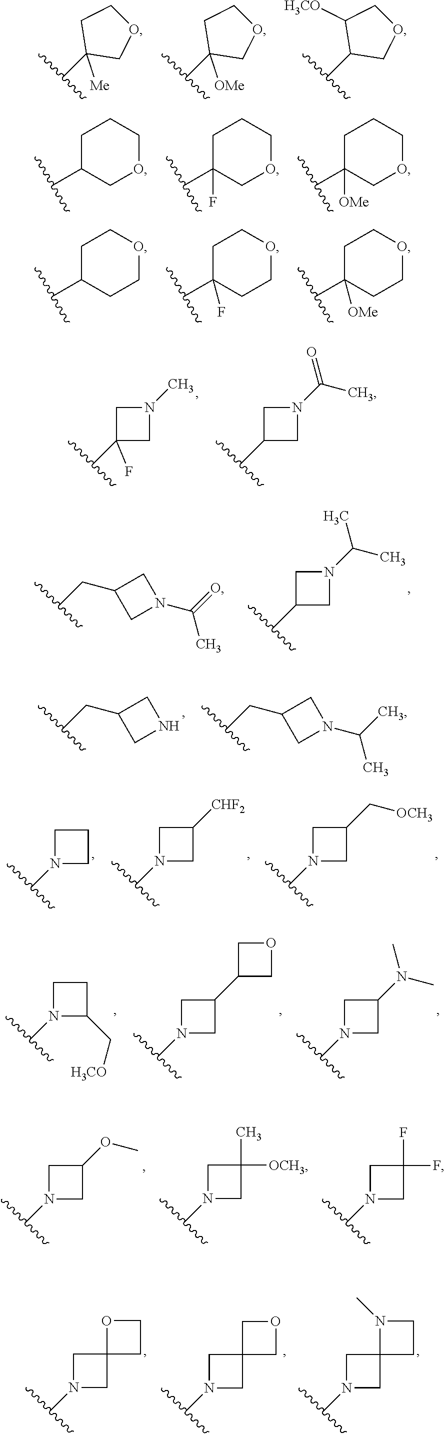

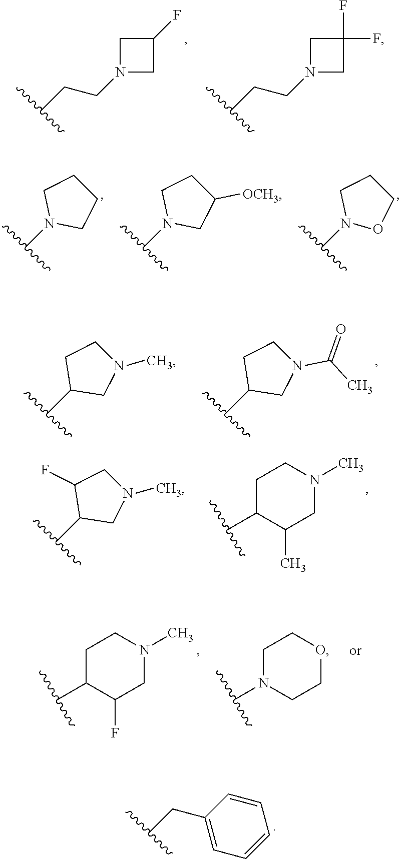

In some cases, each substituent of the heteroaryl independently is CH₃, CH(CH₃)₂, C(CH₃)₂OH, CH₂OCD₃, CH₂CH₂OCH₃, CF₂CH₂OCH₃, CH₂CH₂OCD₃, CH₂CH₂CH₂OCH₃, CH₂CH(CH₃)OCH₃, CH₂C(CH₃)₂OCH₃,

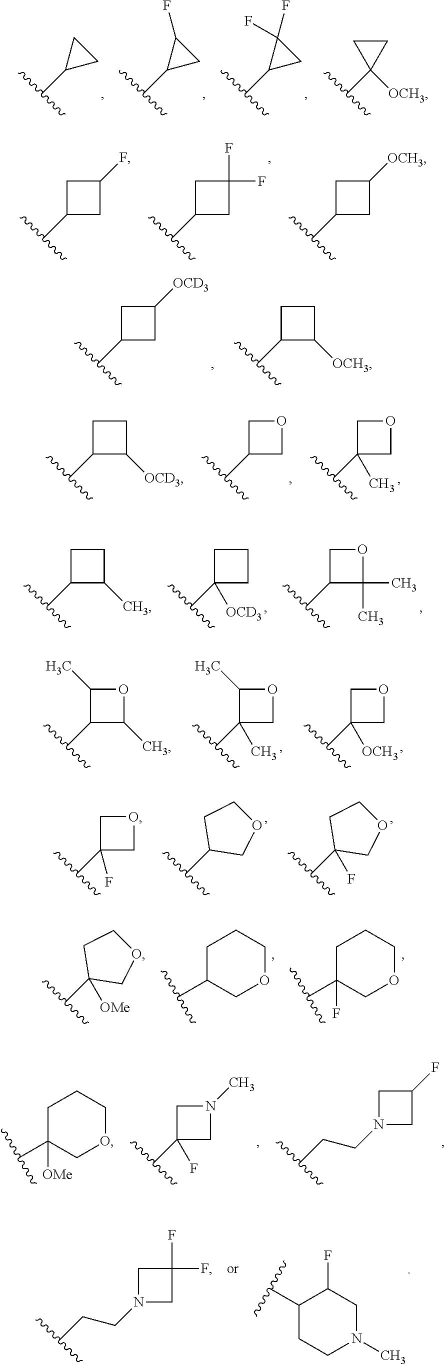

In some cases, each substituent of the heteroaryl independently is CH₃, CH(CH₃)₂, C(CH₃)₂OH, CH₂OCD₃, C(CH₃)₂CH₂OH, CH₂CH₂OCH₃, CF₂CH₂OCH₃, CH₂CH₂OCD₃, CH(CH %)OCH₃, CH₂CH₂CH₂OCH₃, CH₂CH(CH₃)OCH₃, CH₂C(CH₃)₂OCH₃,

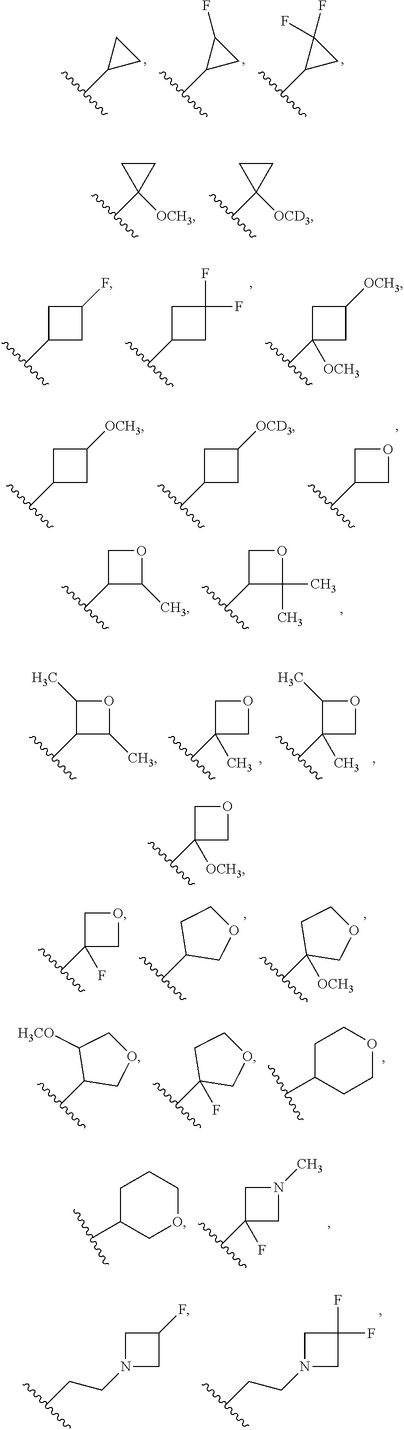

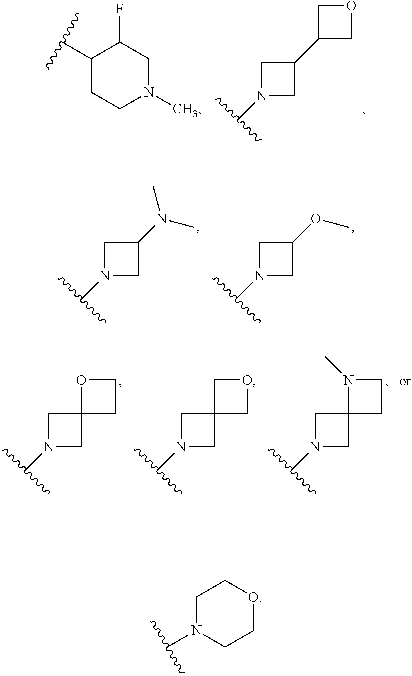

In some cases, each substituent of the heteroaryl independently is CH₃, CH₂CH₂OCH₃, CF₂CH₂OCH₃, CH₂CH₂OCD₃, CH₂CH₂CH₂OCH₃, CH₂CH(CH₃)OCH₃, CH₂C(CH₃)₂OCH₃,

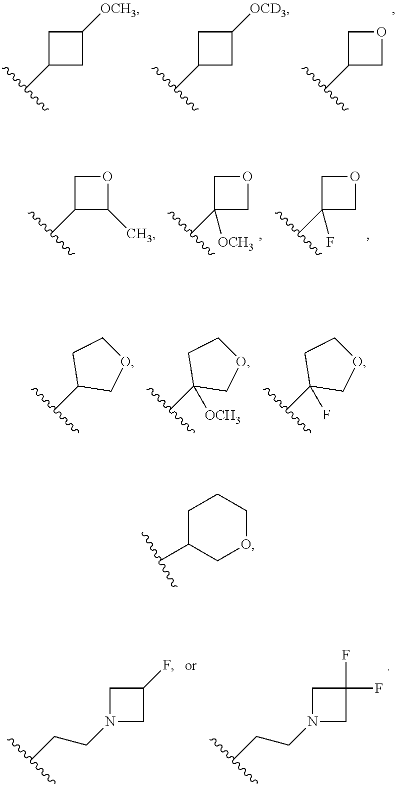

In some cases, each substituent of the heteroaryl independently is CH₃, C(CH₃)₂CH₂OH, CH₂CH₂OCH₃, CH₂CH₂OCD₃, CH(CH₃)OCH₃,

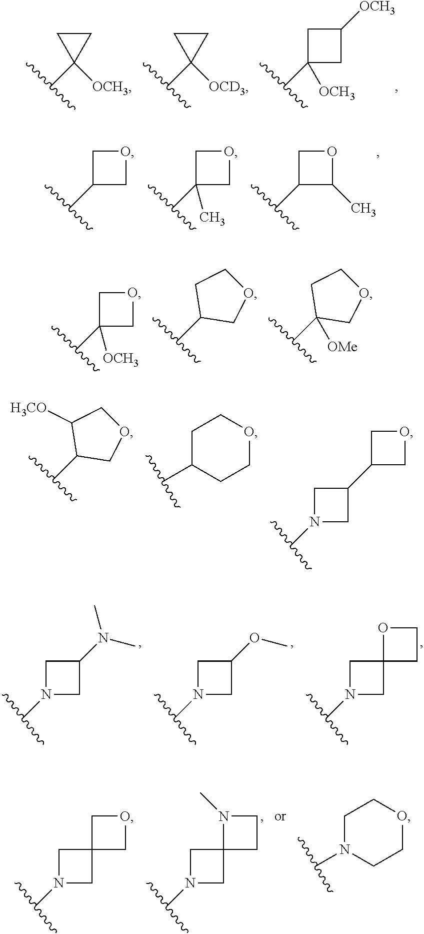

or any combination of the foregoing. In some cases, Z is heteroaryl that is substituted with CH₃, CH₂CH₂OCH₃, OCH₃,

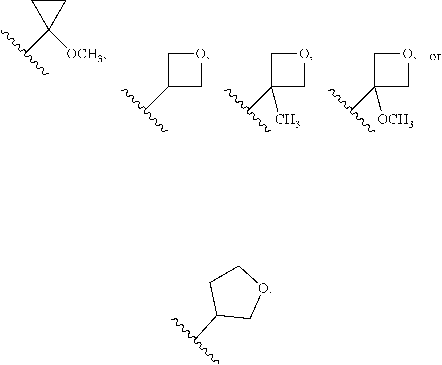

or any combination of the foregoing. In some cases, the heteroaryl of Z is substituted with CH₃ and C(CH₃)CH₂OH, CH₂CH₂OCH₃, CH₂CH₂OCD₃, CH(CH₃)OCH₃,

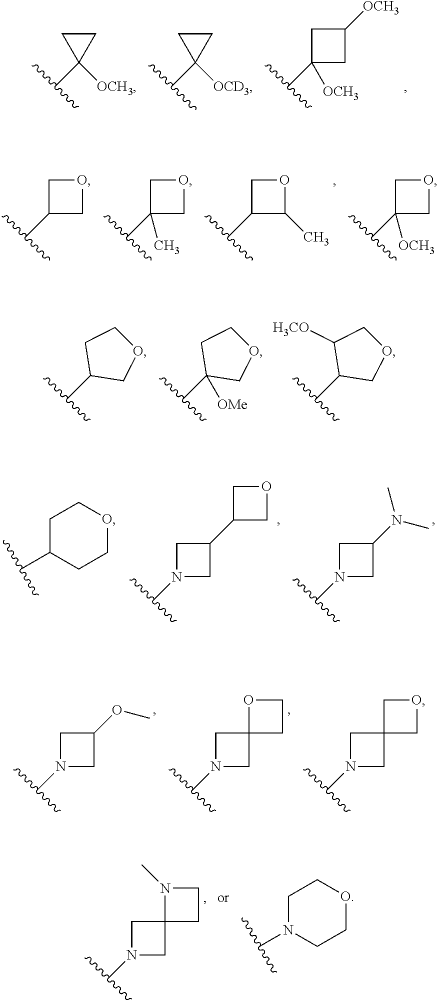

In some cases, each substituent of the heteroaryl independently is CH₃, CH₂CH₂OCH₃.

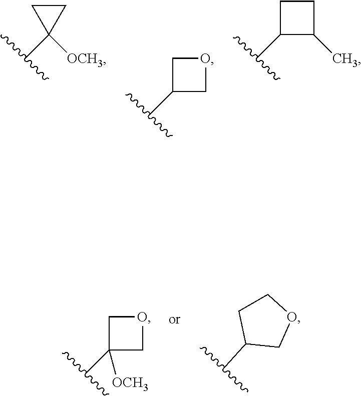 [SMILES: *C1(OC)CC1 ; *C1CCC1C ; *C1COC1 ; *C1(OC)COC1 ; *C1CCOC1]

or any combination of the foregoing. In some cases, the heteroaryl group has 2 substituents selected from CH₃, CH₂CH₂OCH₃,

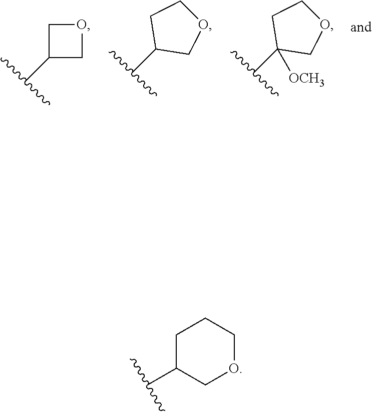 [SMILES: *C1COC1 ; *C1CCOC1 ; *C1(OC)CCOC1 ; *C1CCCOC1]

In some cases, Z is heteroaryl that is substituted with CH₃ and CH₂CH₂OCH₃.

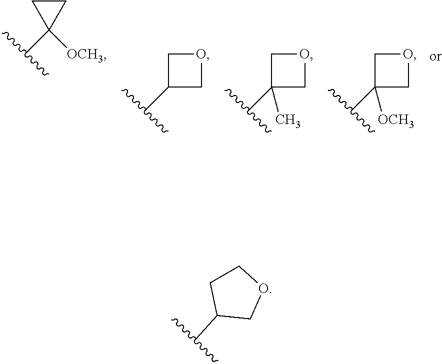

In some cases, Z is heteroaryl and has a structure

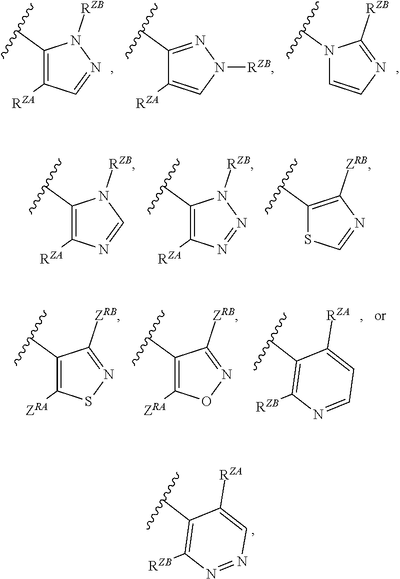

wherein each of R^{ZA} and R^{ZB} is as defined herein for the substituents of the heteroaryl group of Z. In some cases, Z is

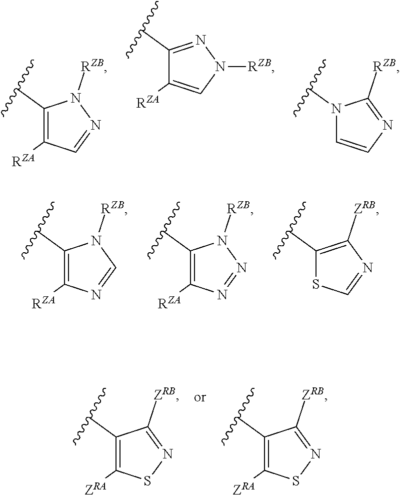

wherein R^{ZA} and R^{ZB} the same as previously defined for the 1-4 substituents of the heteroaryl group of Z. In some cases, Z is heteroaryl and has a structure

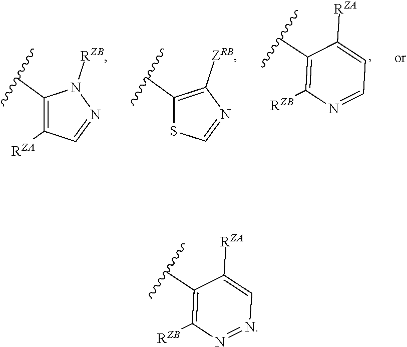

wherein each of R^{ZA} and R^{ZB} is as defined herein for the substituents of the heteroaryl group of Z. In some cases, Z is

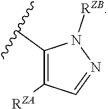

In some cases, Z is

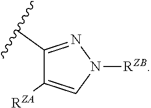

In some cases, Z is

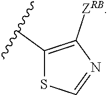

In some cases, Z is

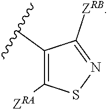

In some cases. Z is

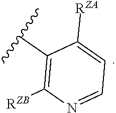

In some cases, Z is

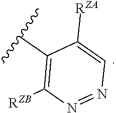

In some cases, each of R^{ZA} and R^{ZB} independently is Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃, CH₂CH₂CH₂OCH₃, CH₂CH₂CH₂OCD₃, CH(CH₃)OCH₃, CH(CH₃)CH₂OCH₃, CH(OCH₃)CH₂OCH₃, CH(CH₃)(OCH₃)CH₂OCH₃, CH(CH₂F)(CH₃)CH₂OCD₃, CH(CH₃)CH₂OCD₃, C(CHO)₂OCH₃, C(CHO)CH₂OCH₃, C(CH₃)₂CH₂OCD₃, CH₂CH(CH₃)OCH₃, CH(CH₃)₂OCH)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

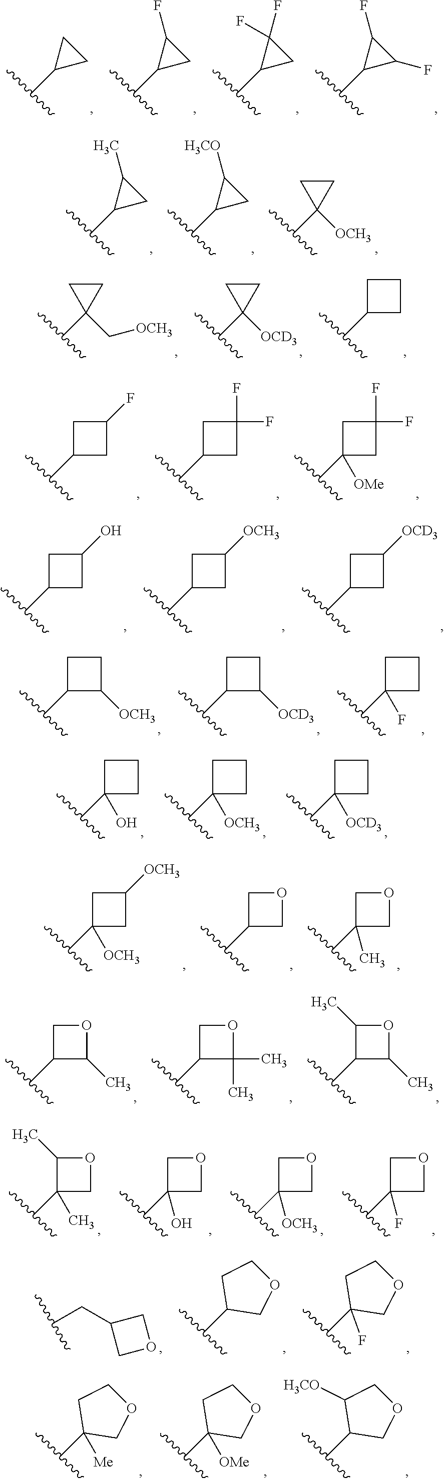

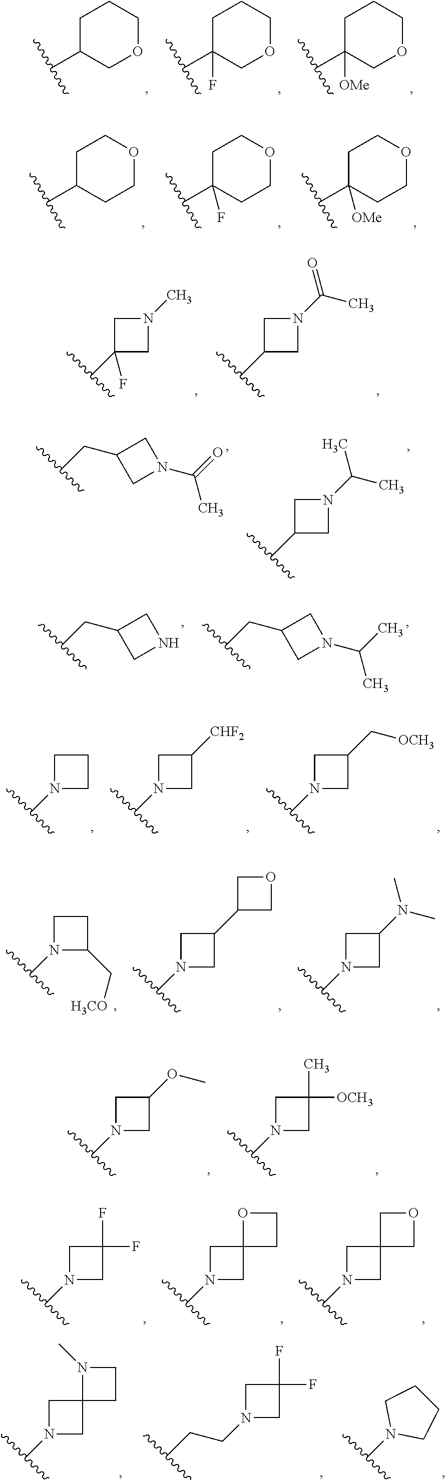

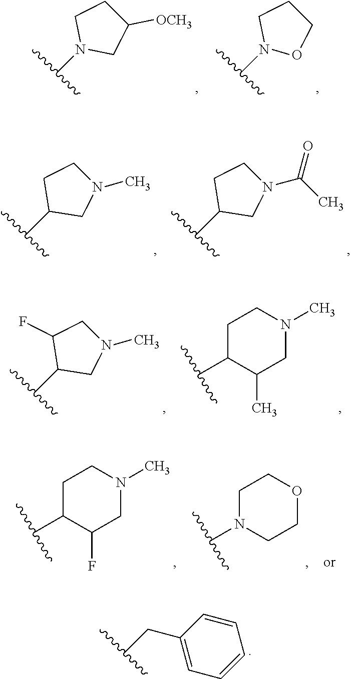

In some cases, R^{ZA} is Cl, F, CH₃, CH₂CH₃, CH(CH₃)₂, CF₃, CHF₂, CH₂F, CH₂CHF₂, or CH₂CH₂F; and R^{ZB} is CH₃, CH₂CH₂OCH₃, CF₂CH₂OCH₃, CH₂CH₂OCD₃, CH₂CH₂CH₂OCH₃, CH₂CH(CH₃)OCH₃, CH₂C(CH₃)₂OCH₃,

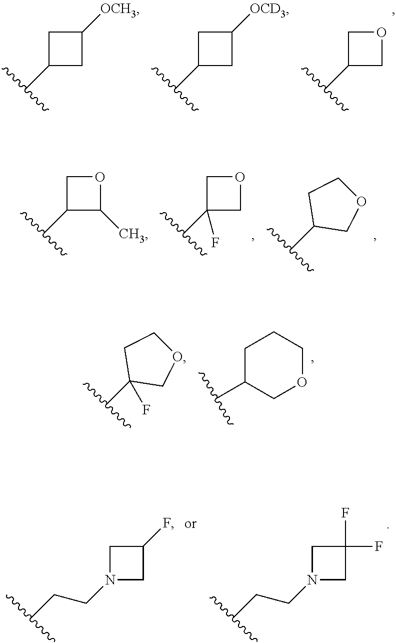

In some cases, R^{ZA} is CH₃; and R^{ZB} is CH₃, CH₂CH₂OCH₃, CF₂CH₂OCH₃, CH₂CH₂OCD₃, CH₂CH₂CH₂OCH₃, CH₂CH(CH₃)OCH₃, CH₂C(CH₃)₂OCH₃.

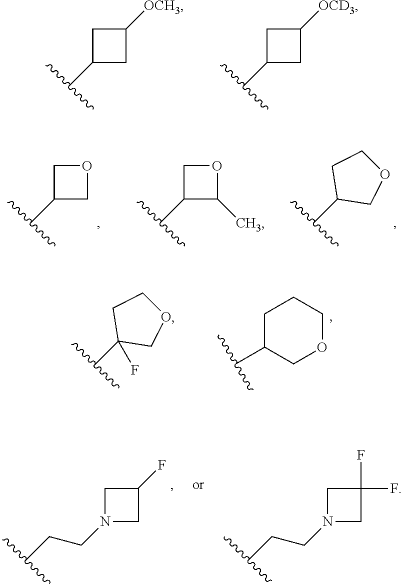

In some cases, R^{ZA} is CH₃; and R^{ZB} is CH₃, C(CH₃)₂CH₂OH, CH₂CH₂OCH₃, CH₂CH₂OCD₃, CH(CH₃)OCH₃,

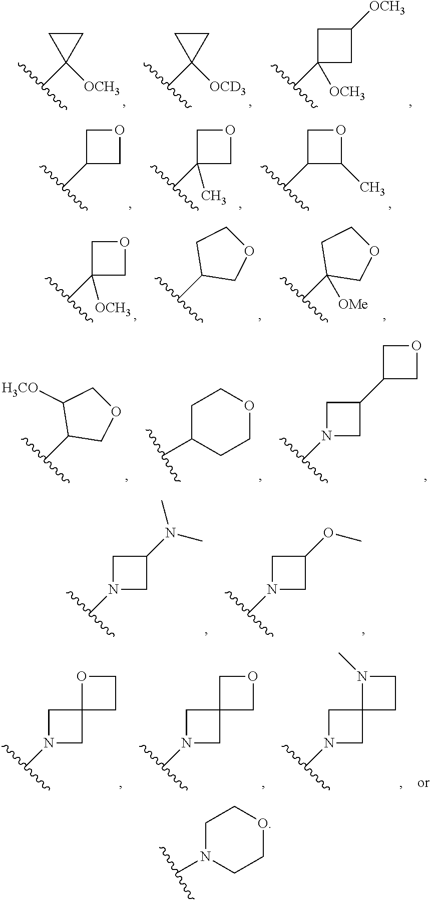

In some cases, R^{ZA} is CH₃; and R^{ZB} is CH₃, CH₂CH₂OCH₃,

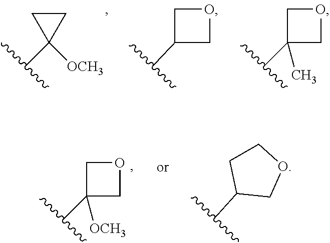

In some cases, Z is

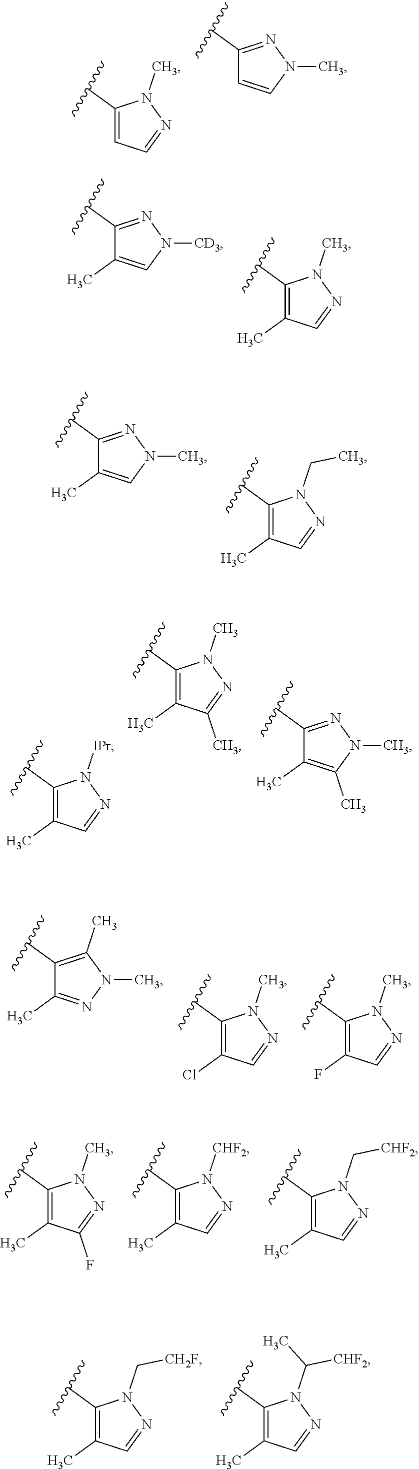

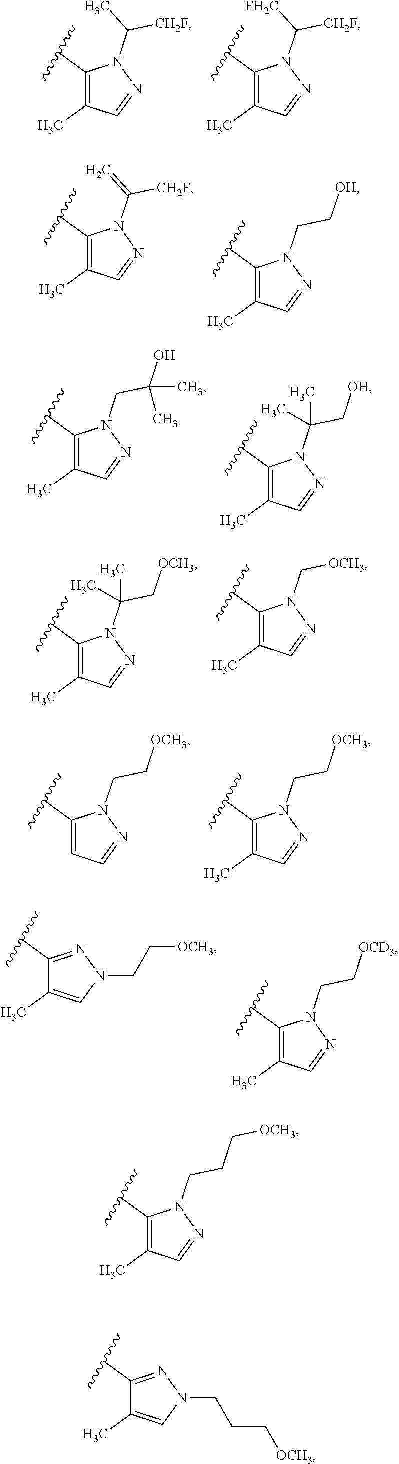

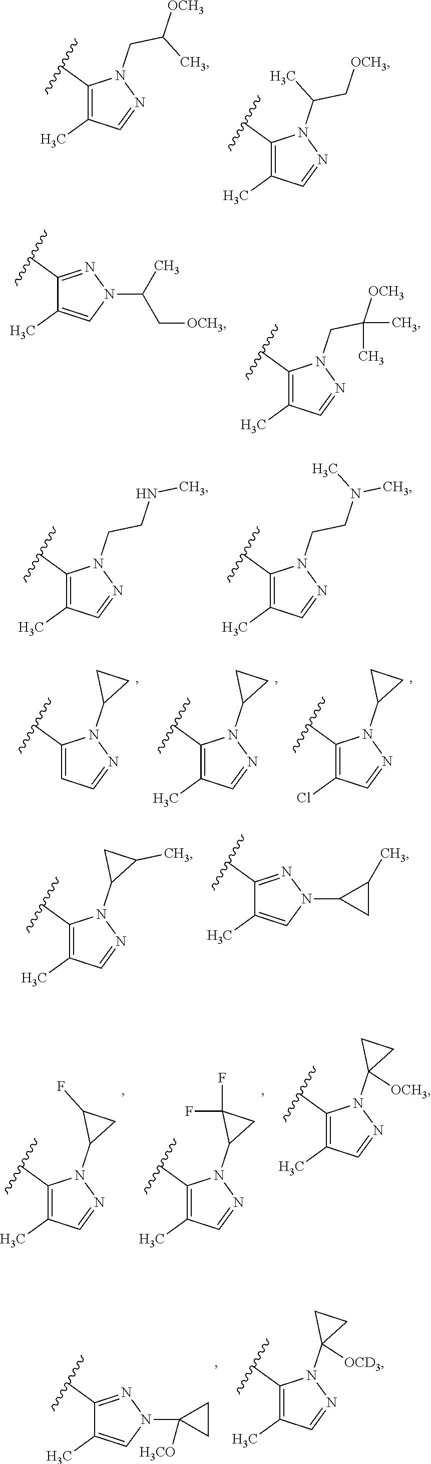

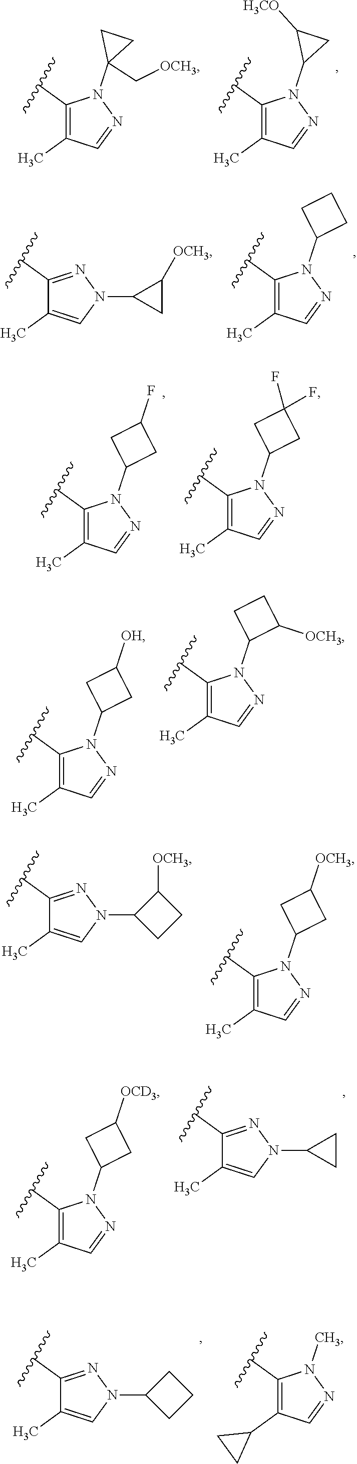

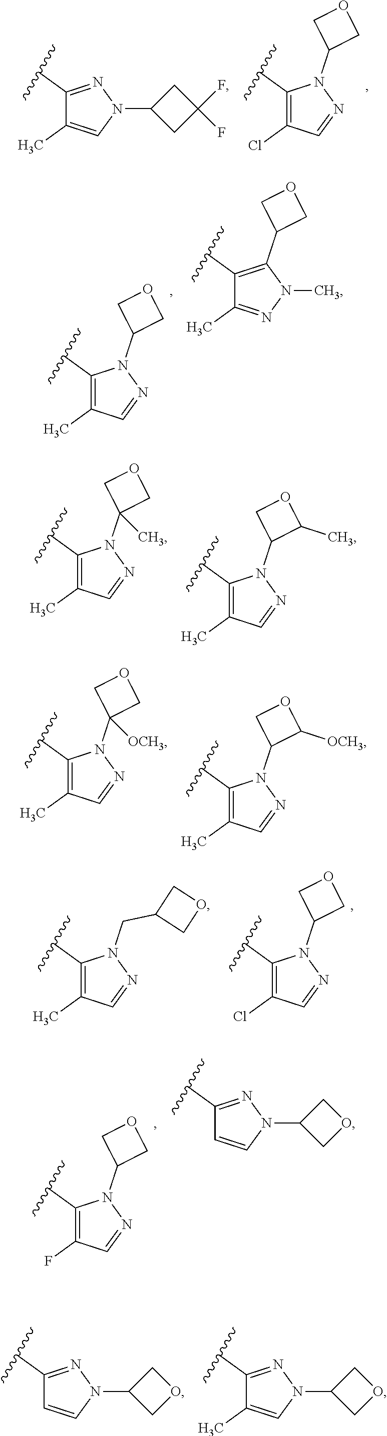

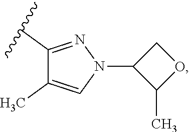 [SMILES: *c1nn(C2COC2C)cc1C]

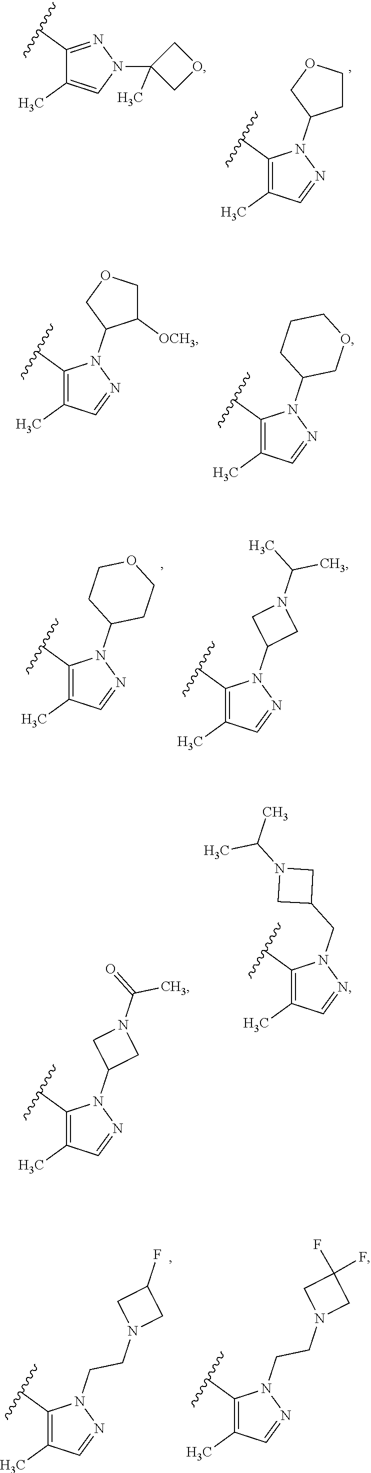

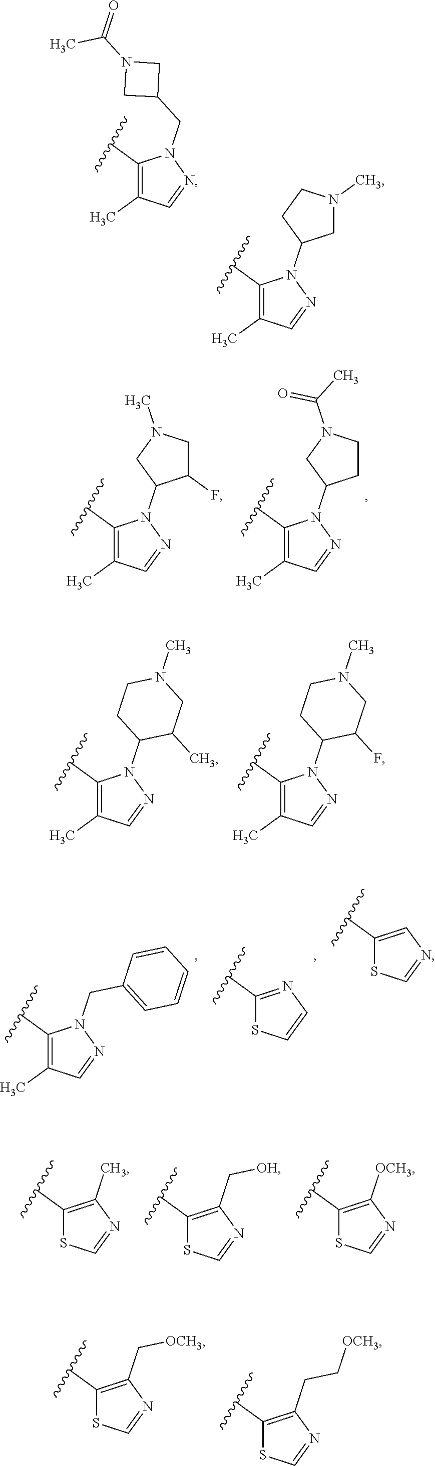

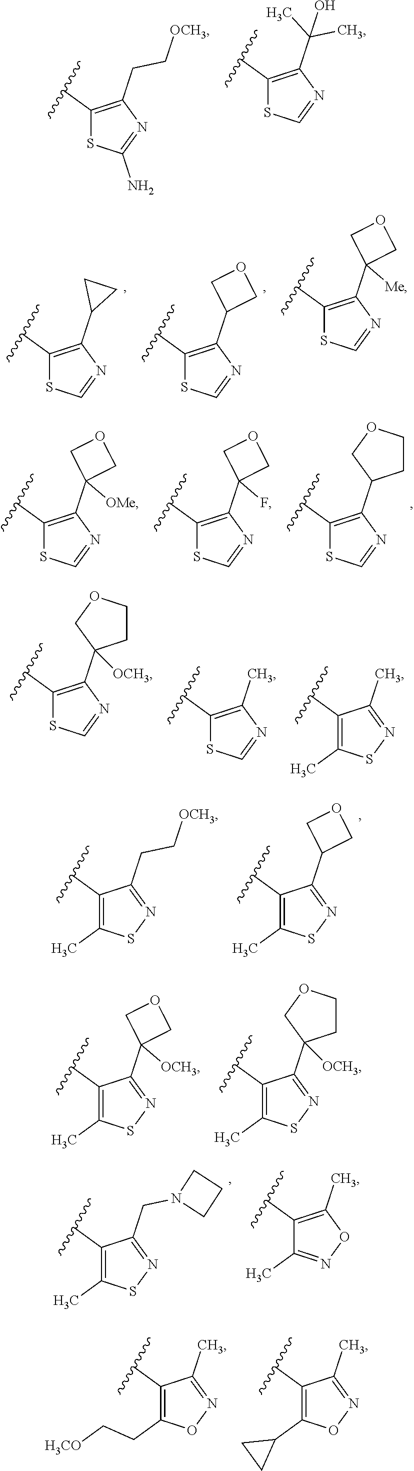

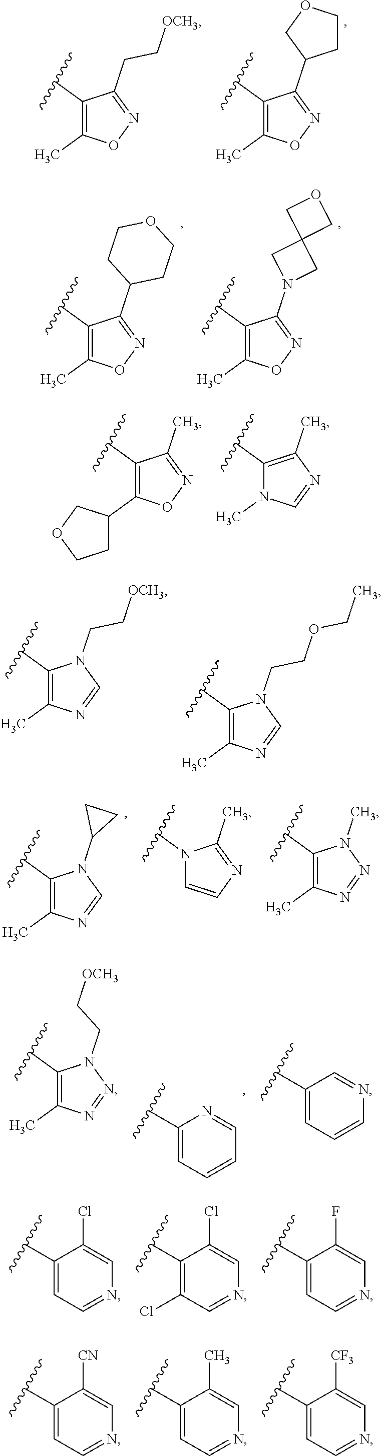

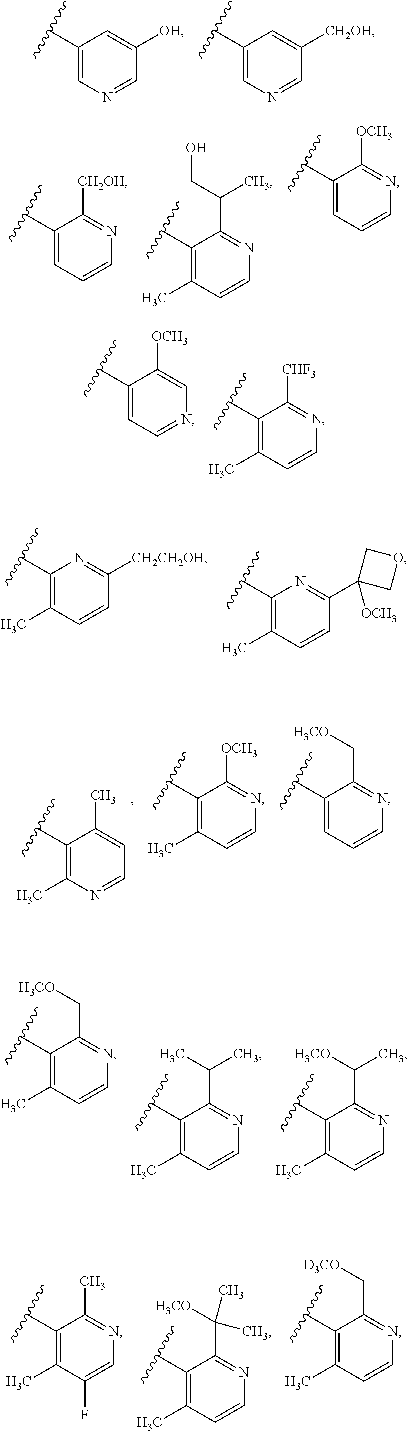

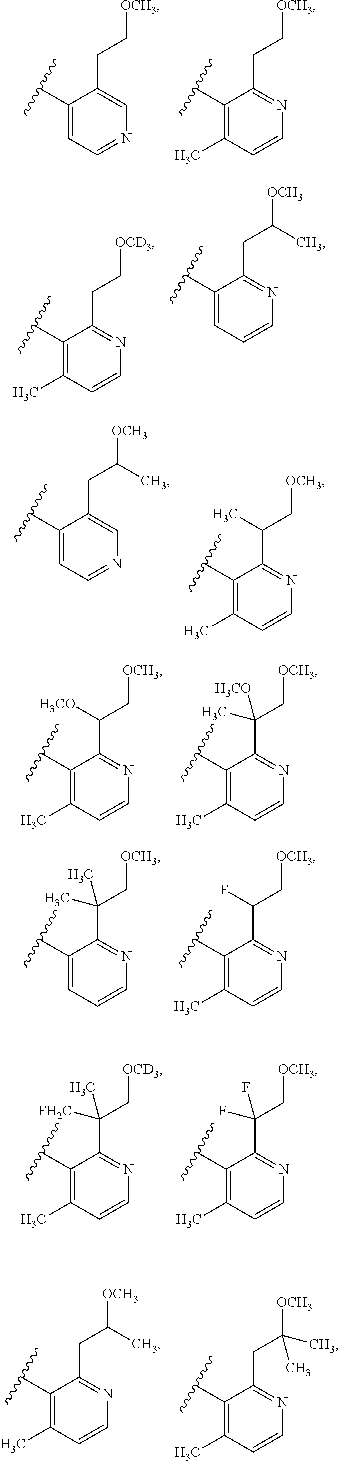

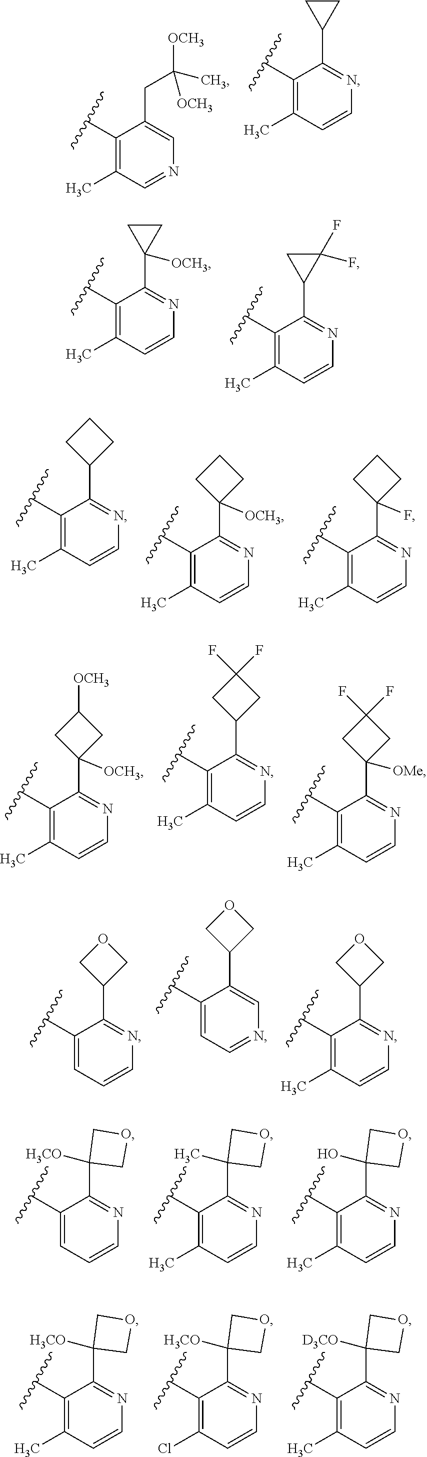

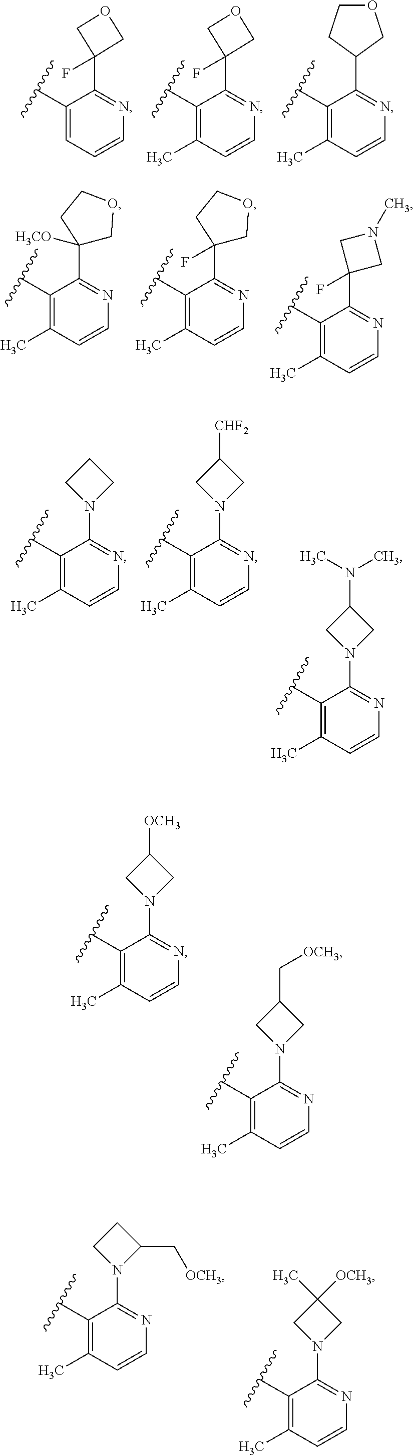

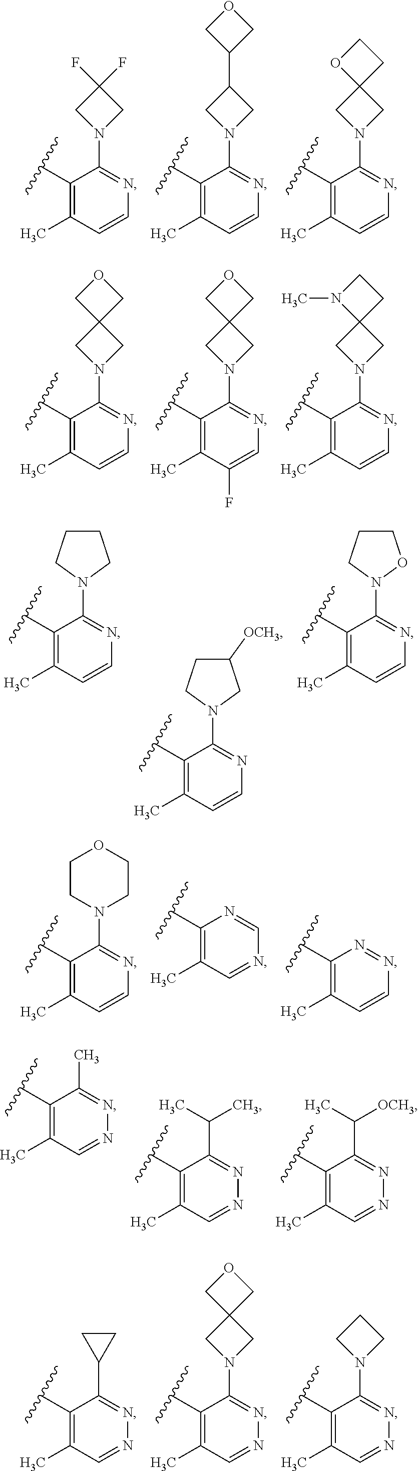

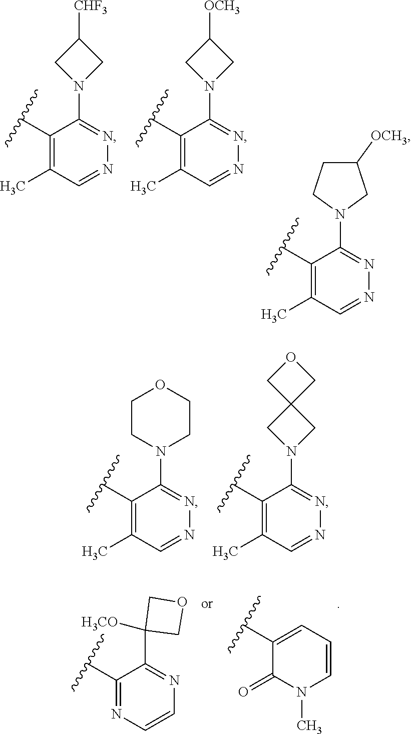

In some cases, Z is

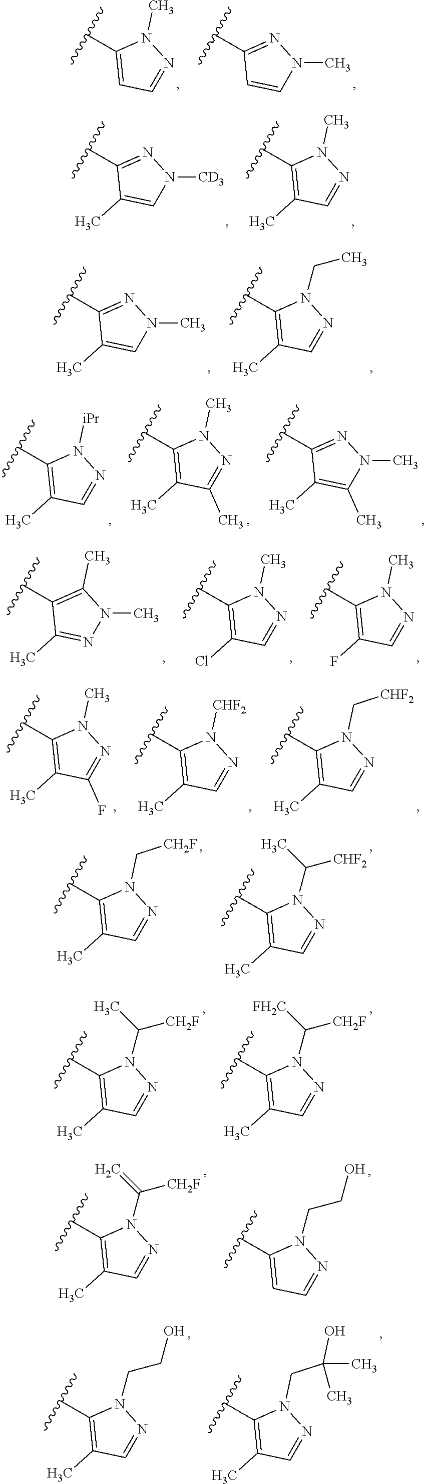

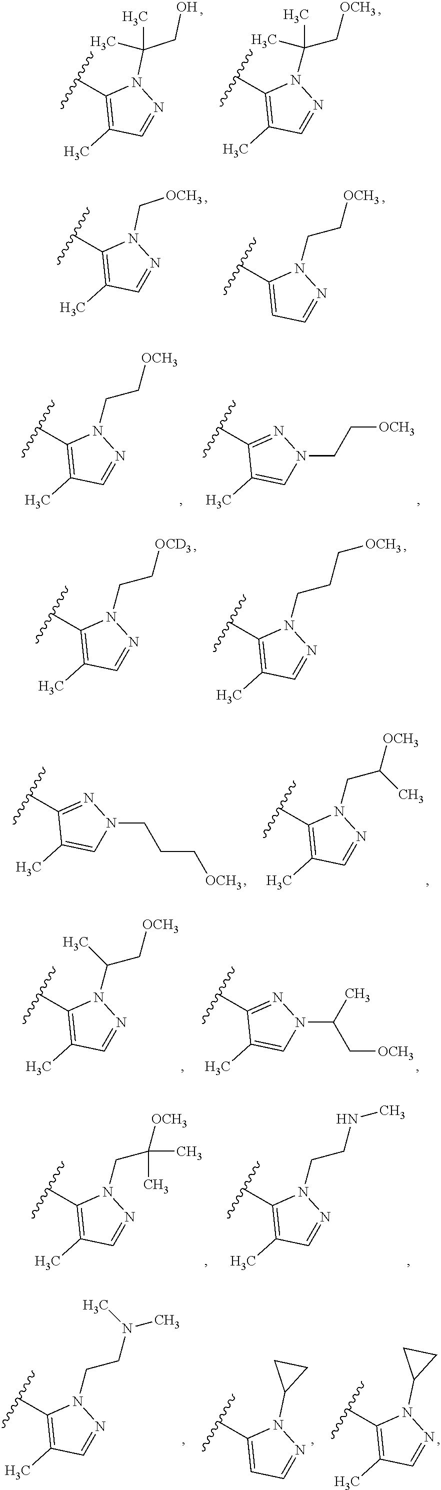

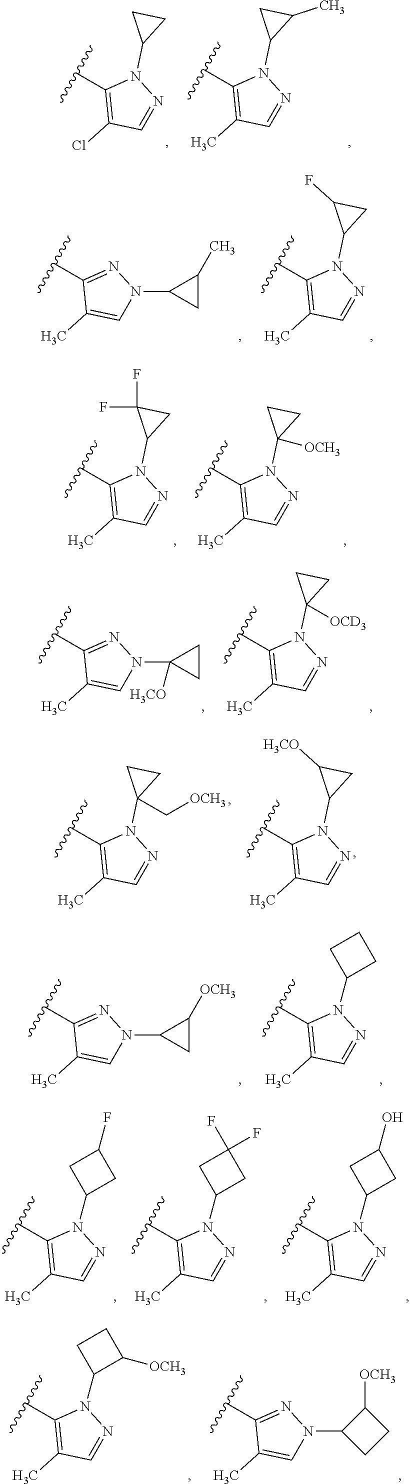

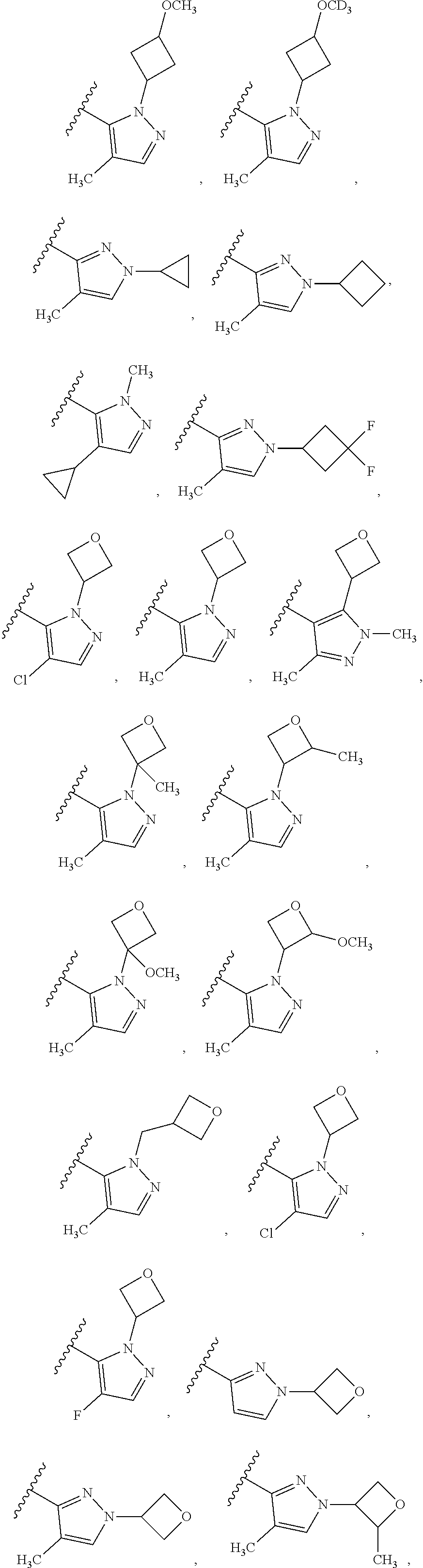

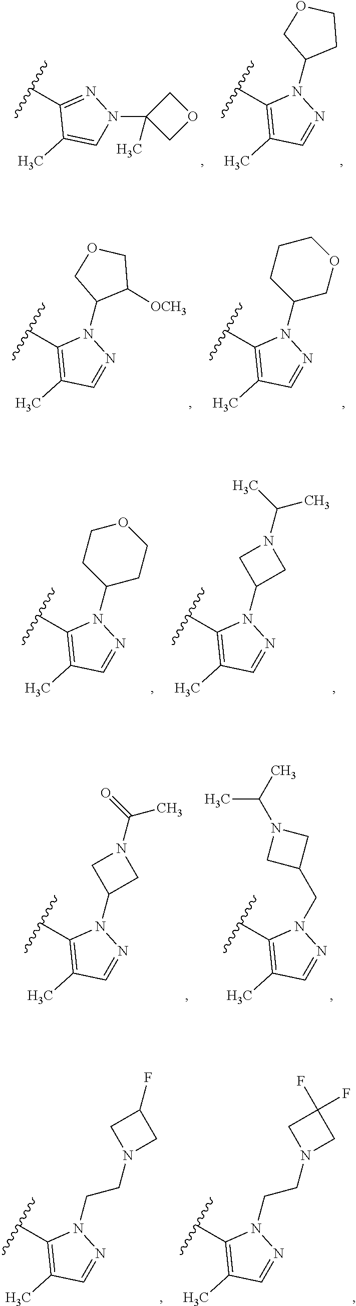

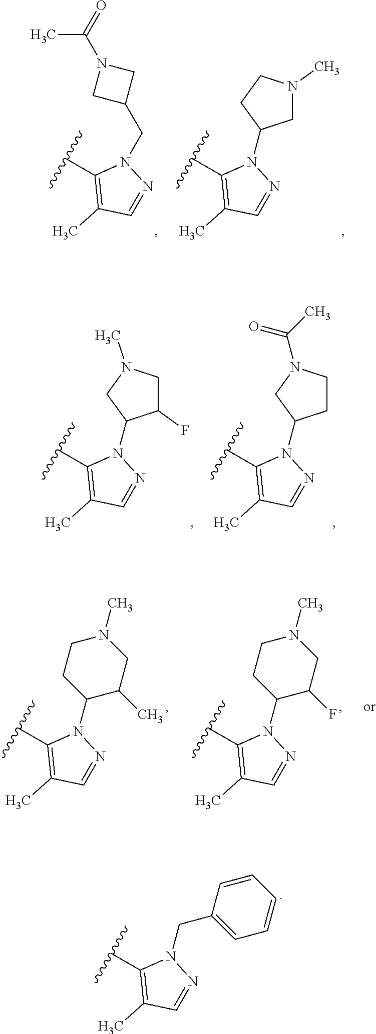

In some cases, Z is

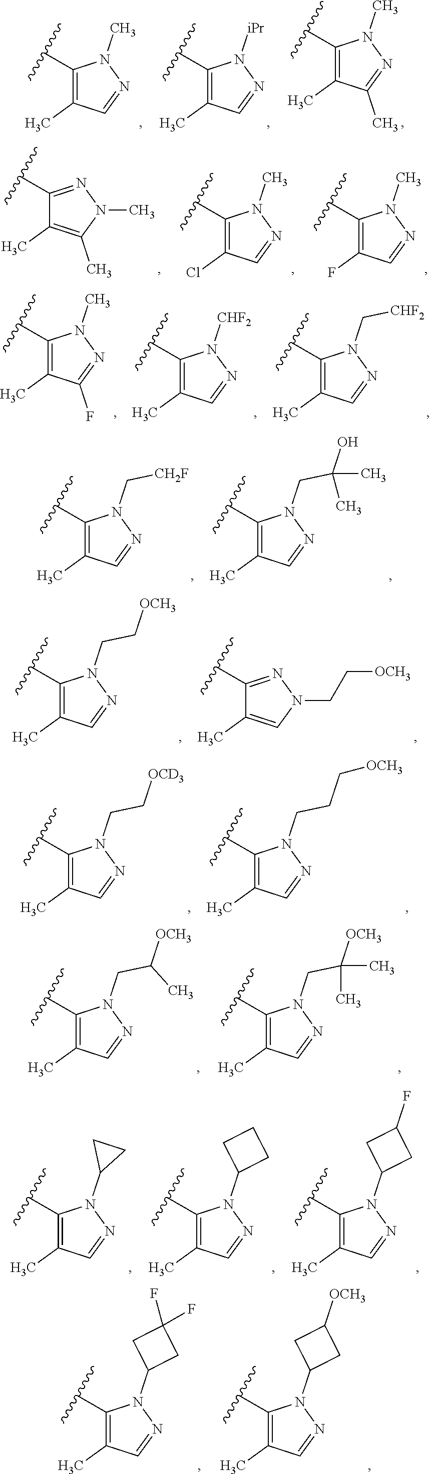

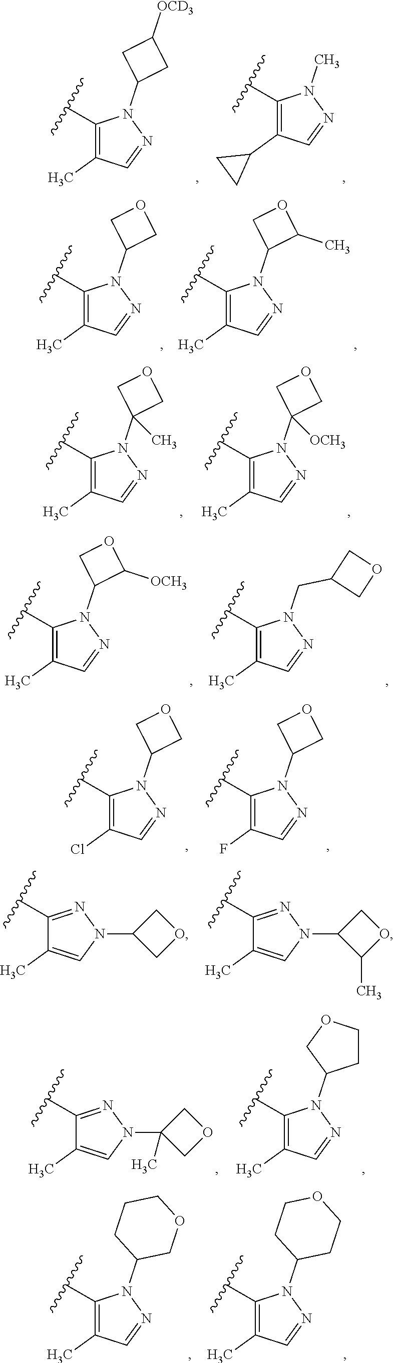

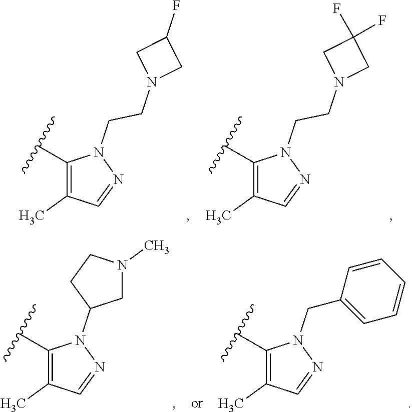 [SMILES: *c1c(C)cnn1CCN1CC(F)C1 ; *c1c(C)cnn1CCN1CC(F)(F)C1 ; *c1c(C)cnn1C1CCN(C)C1 ; *c1c(C)cnn1Cc1ccccc1]

In some cases, Z is

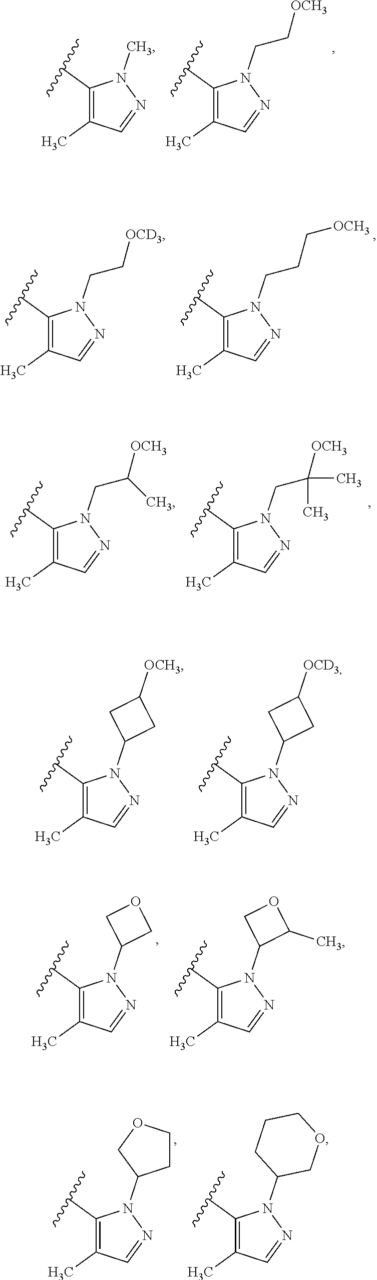

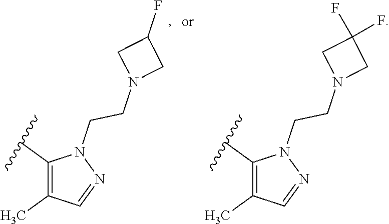 [SMILES: *c1c(C)cnn1CCN1CC(F)C1 ; *c1c(C)cnn1CCN1CC(F)(F)C1]

In some cases, Z is

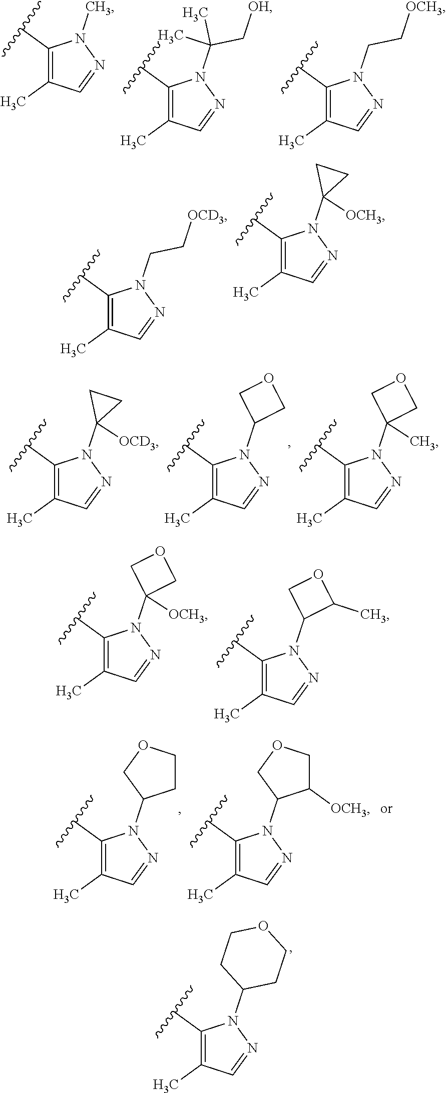

In some cases, Z is

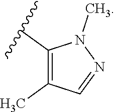 [SMILES: *c1c(C)cnn1C]

In some cases, Z is

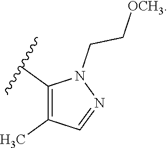 [SMILES: *c1c(C)cnn1CCOC]

In some cases, Z is

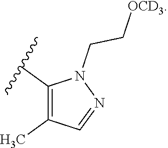

In some cases, Z is

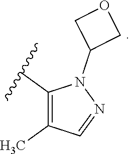 [SMILES: *c1c(C)cnn1C1COC1]

In some cases, Z is

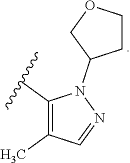 [SMILES: *c1c(C)cnn1C1CCOC1]

In some cases, Z is

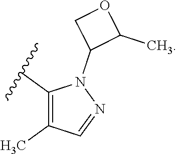 [SMILES: *c1c(C)cnn1C1COC1C]

In some cases, Z is

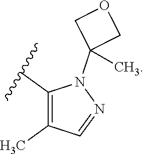 [SMILES: *c1c(C)cnn1C1(C)COC1]

In some cases, Z is

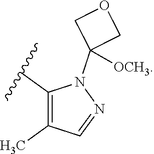 [SMILES: *c1c(C)cnn1C1(OC)COC1]

In some cases, Z is

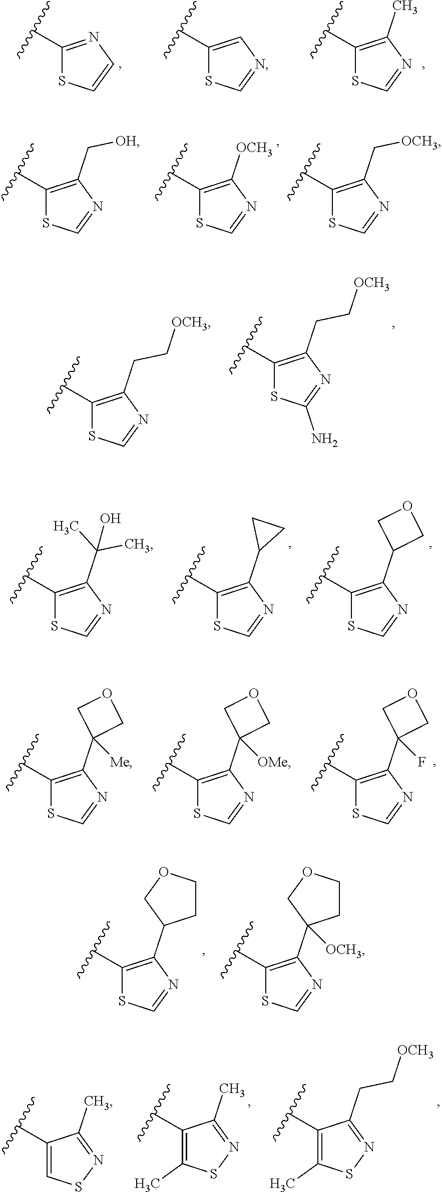

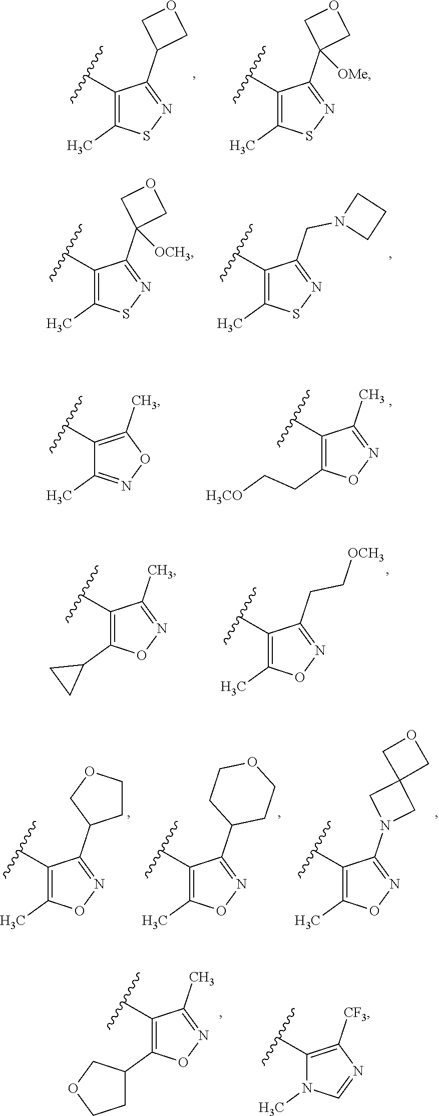

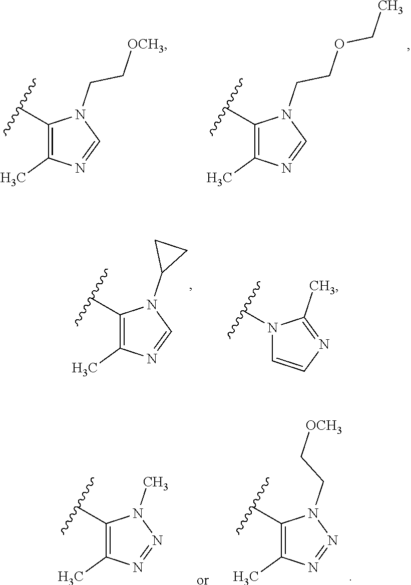

In some cases, Z is

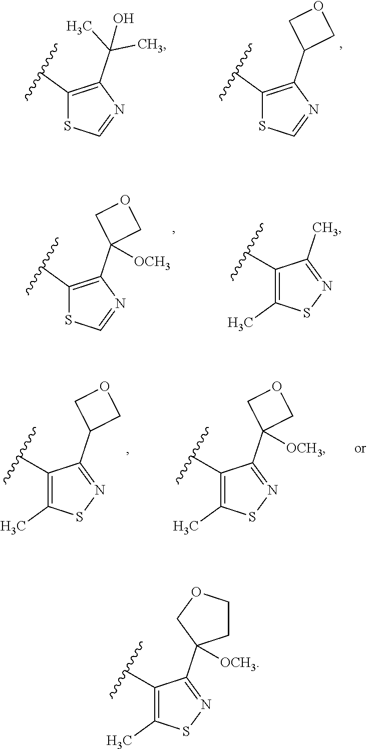

In some cases, Z is

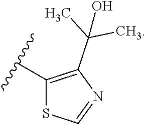 [SMILES: *c1scnc1C(C)(C)O]

In some cases, Z is

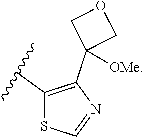 [SMILES: *c1scnc1C1(OC)COC1]

In some cases, Z is

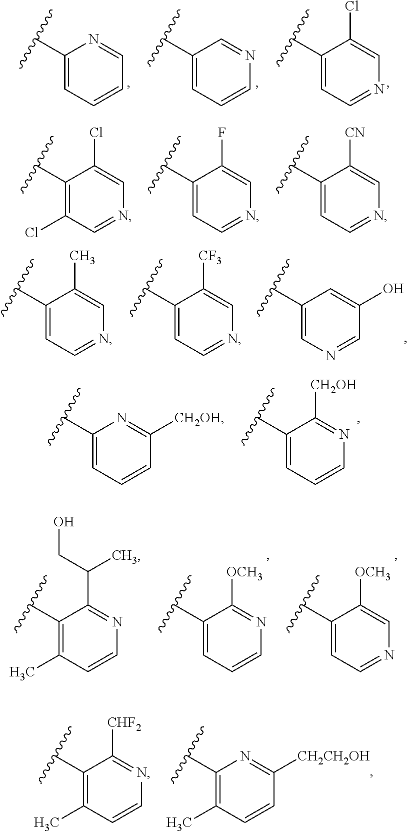

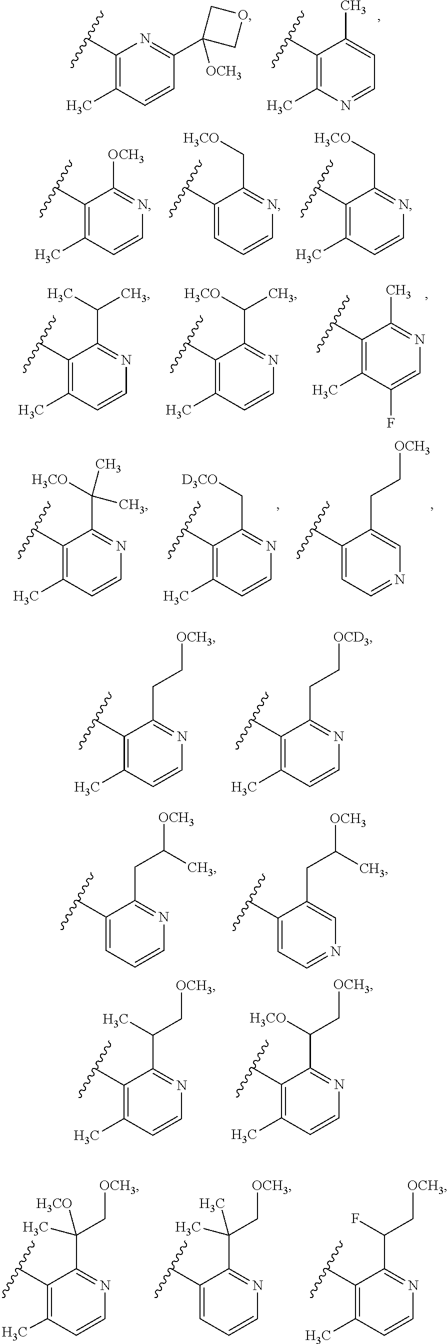

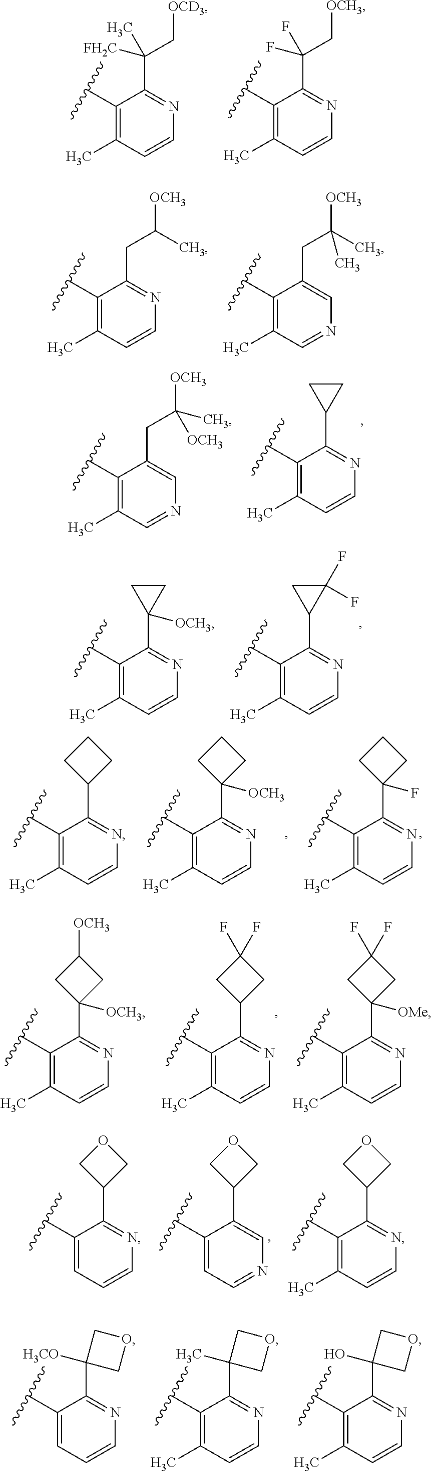

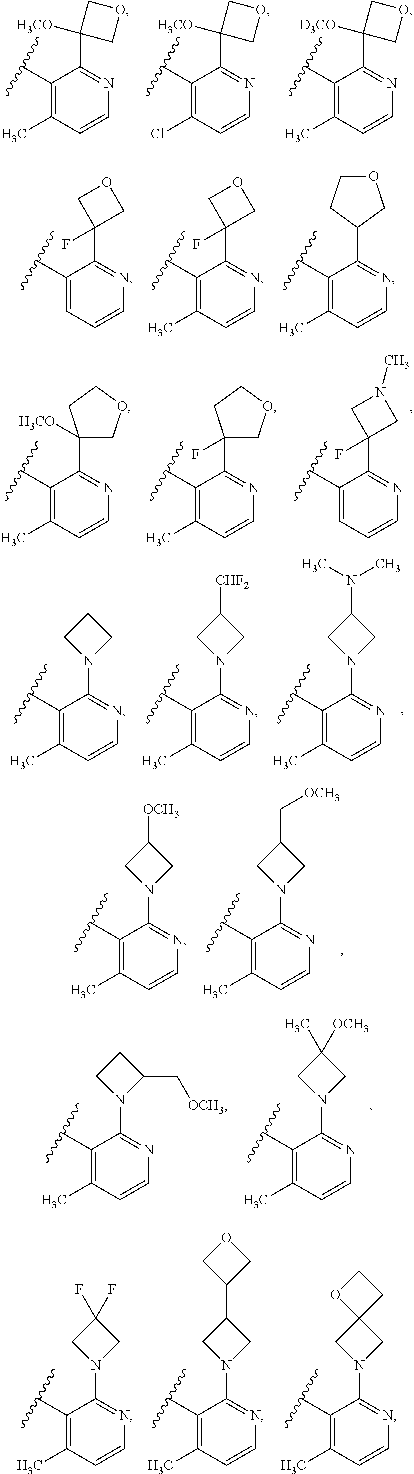

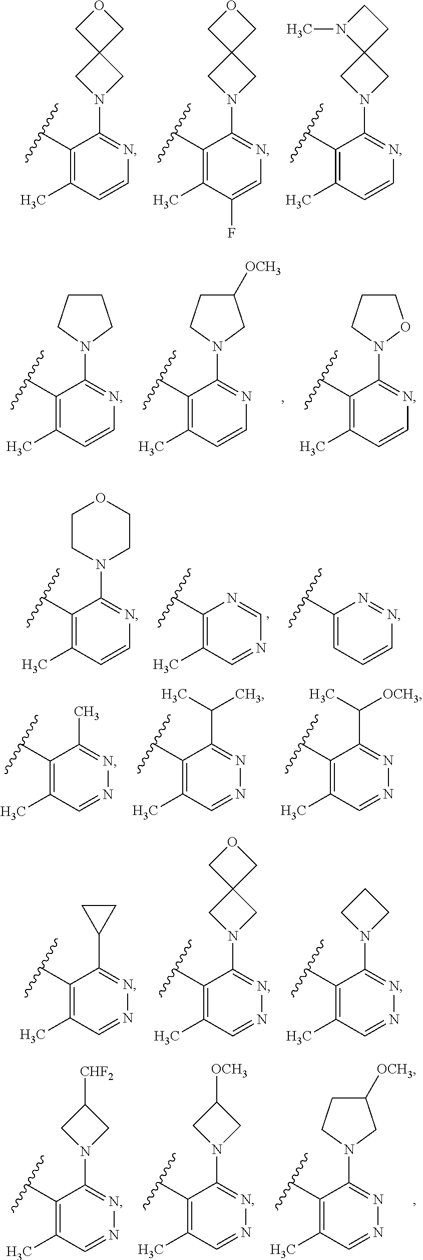

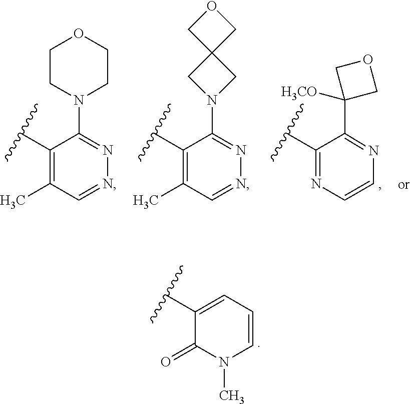

In some cases, Z is

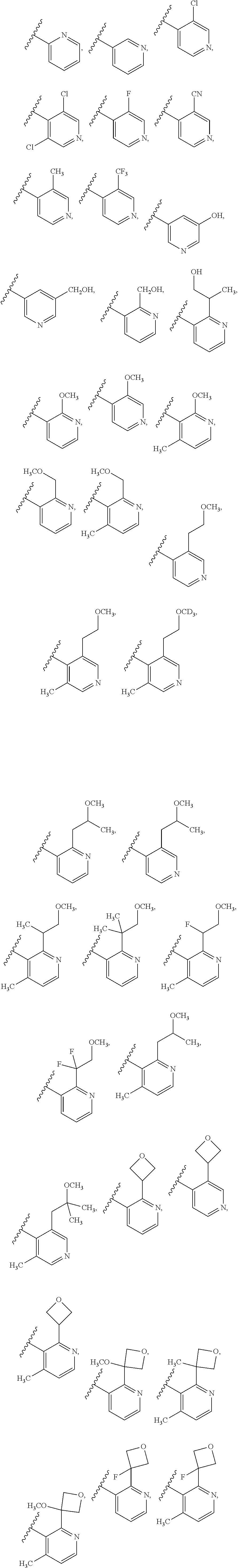

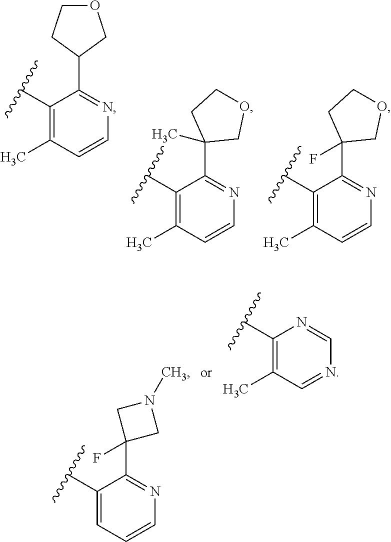

In some cases, Z is

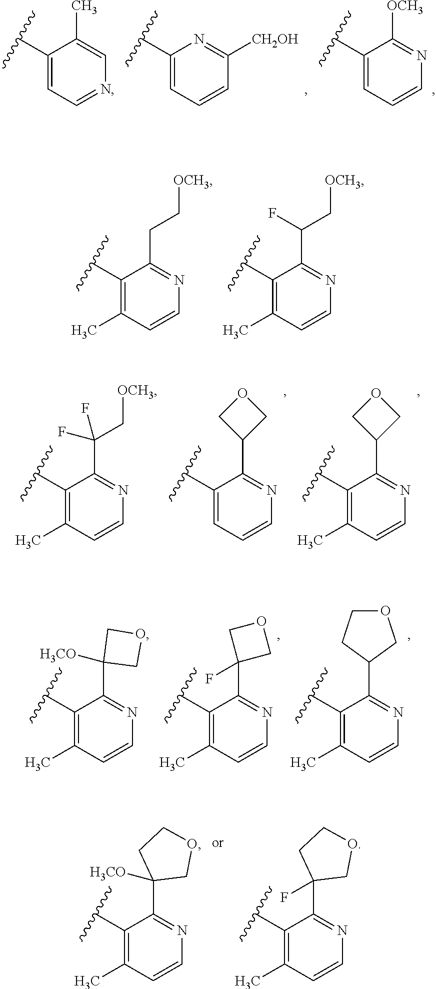

In some cases, Z is

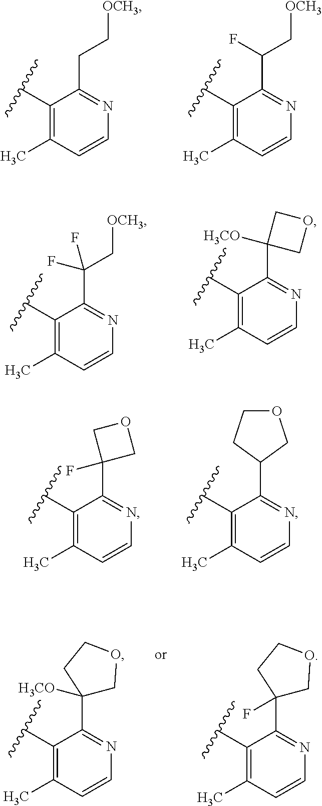

In some cases, Z is

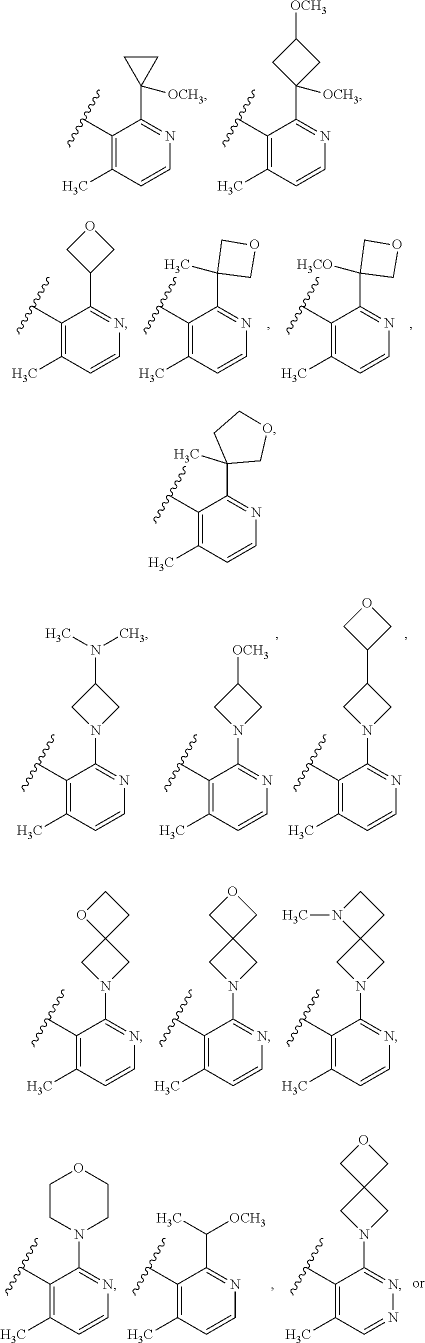

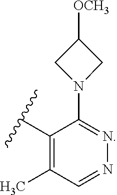 [SMILES: *c1c(C)cnnc1N1CC(OC)C1]

In some cases, Z is

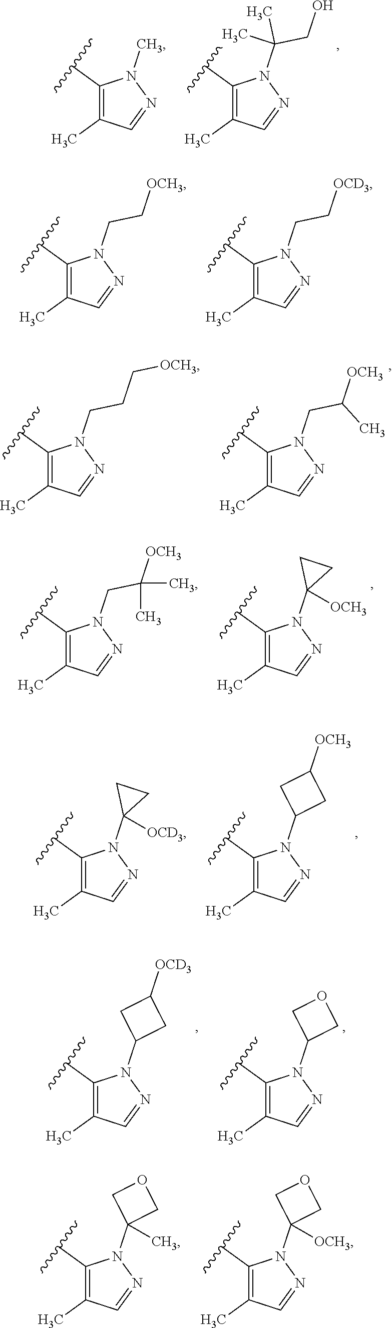

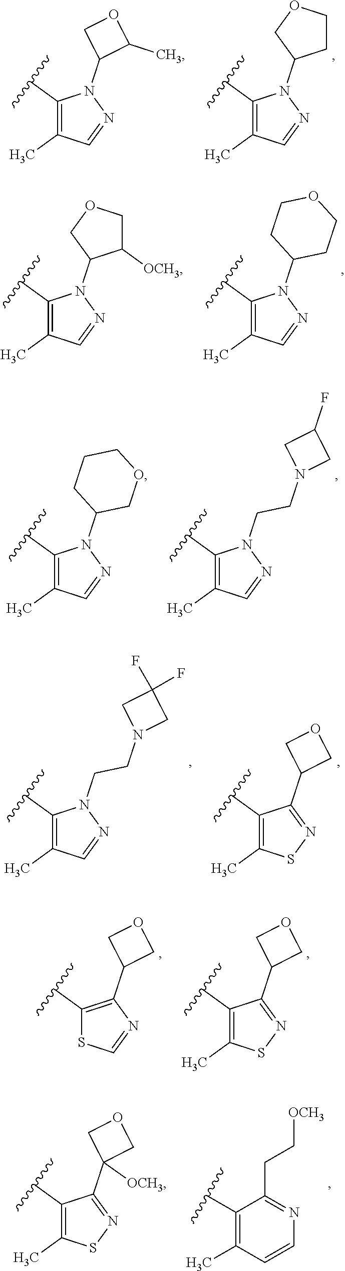

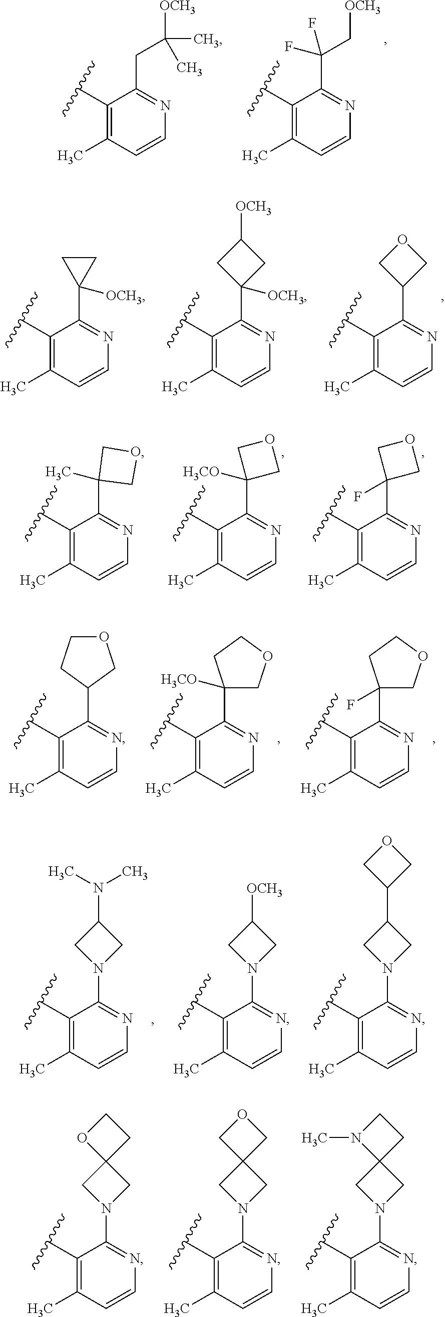

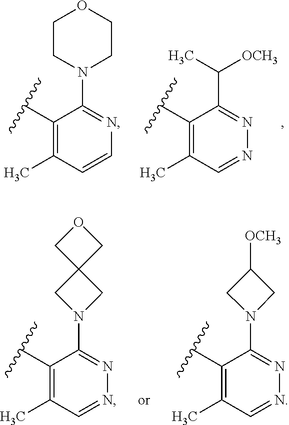 [SMILES: *c1c(C)ccnc1N1CCOCC1 ; *c1c(C)cnnc1C(C)OC ; *c1c(C)cnnc1N1CC2(COC2)C1 ; *c1c(C)cnnc1N1CC(OC)C1]

In some cases, Z is

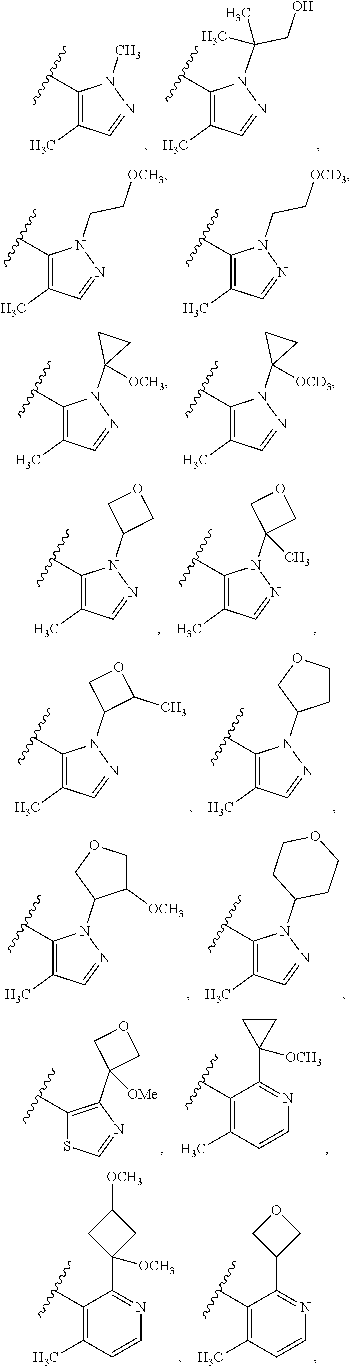

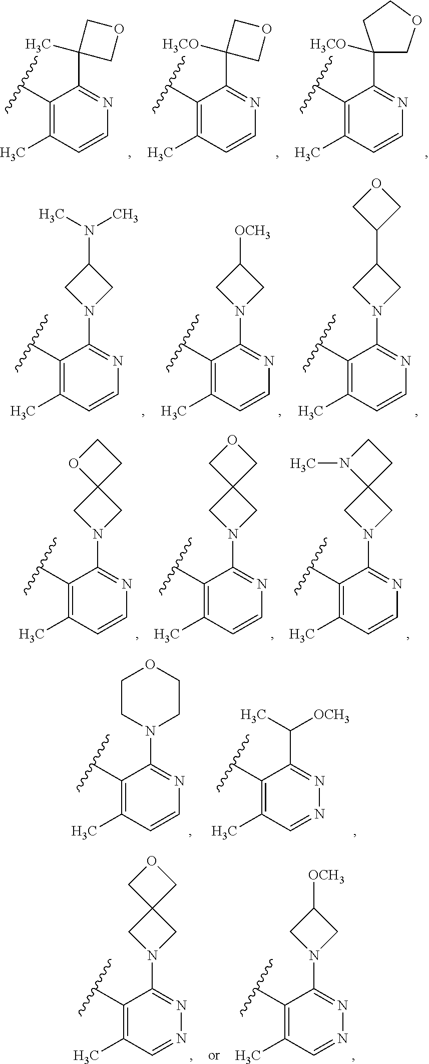

In some cases, Z is

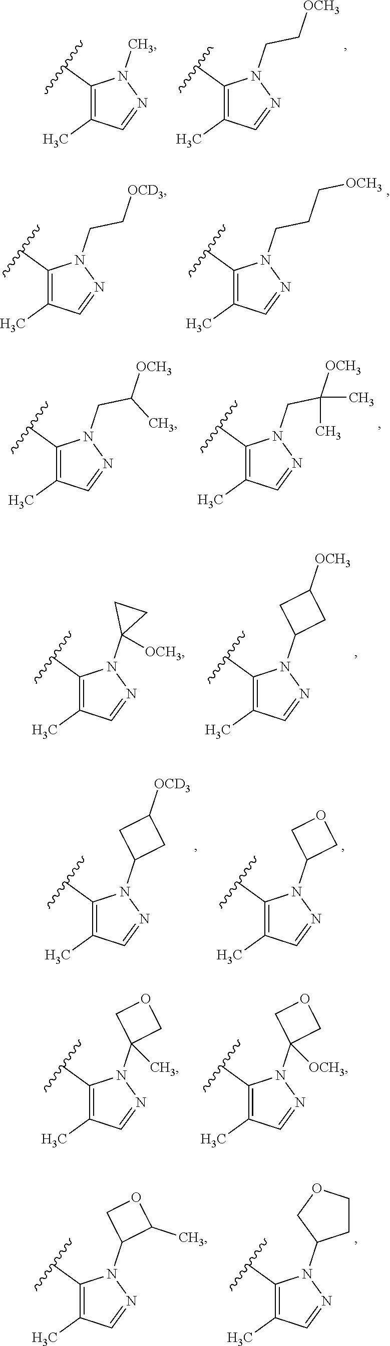

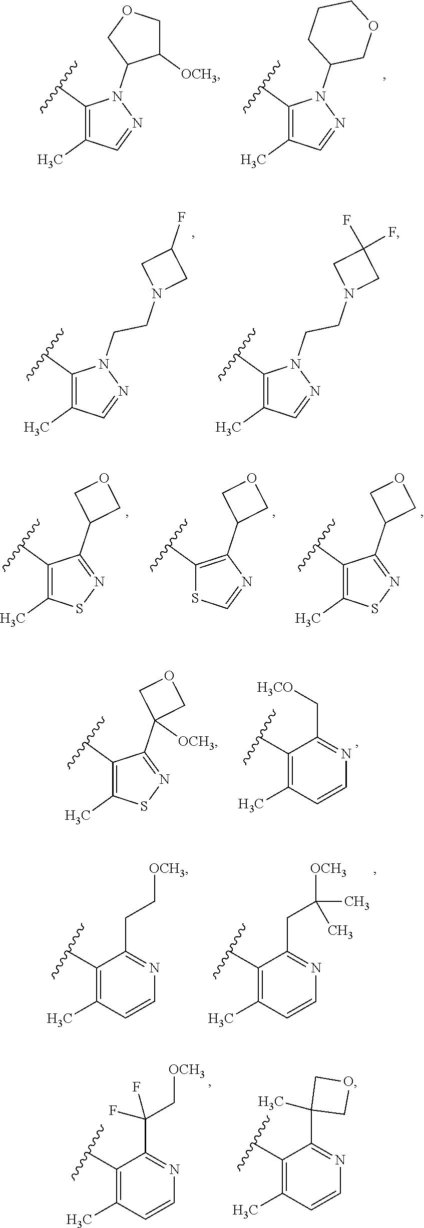

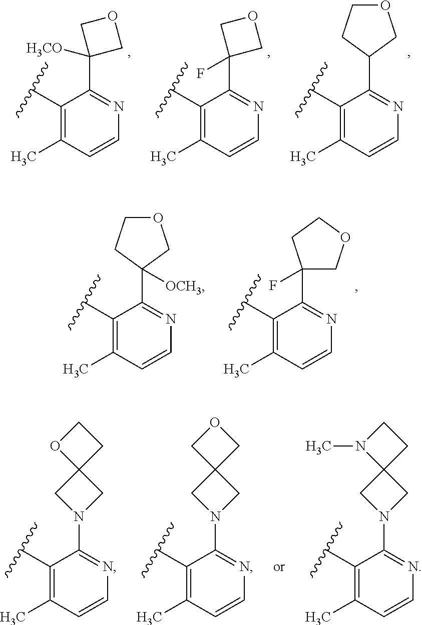

In some cases, Z is

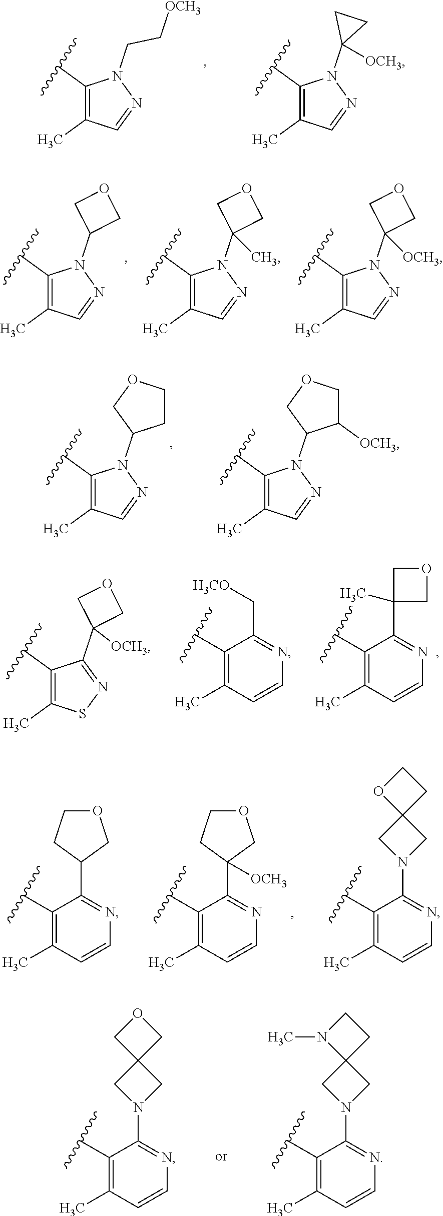

In some cases, Z is

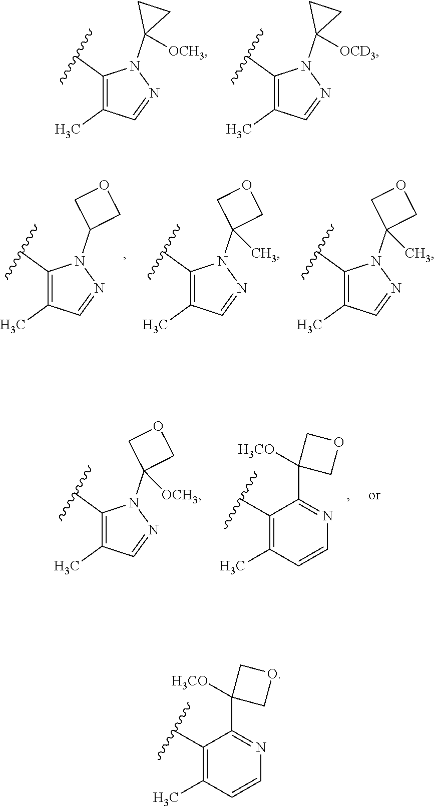

In some cases, Z is a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein the bicyclic ring is optionally substituted with 1-4 substituents. In some cases, the heteroaryl ring of the bicyclic ring is pyridyl, pyridazinyl, pyrimidinyl, or pyrazinyl; the cycloalkyl ring of the bicyclic ring is cyclopentyl or cyclohexyl and the heterocycloalkyl ring of the bicyclic ring is pyrrolidinyl, tetrahydrofuranyl, tetrahydropyranyl, or tetrahydrothiophenyl. In some cases, the heteroaryl group is pyridyl and the heterocycloalkyl group is furanyl. In some cases, Z is a bicyclic ring comprising a heteroaryl ring having 5 or 6 total ring atoms and 1-3 heteroatoms selected from N, O, and S fused to a ring having 5 or 6 total ring atoms and 0, 1, or 2 heteroatoms selected from N, O, and S; wherein the bicyclic ring is optionally substituted with 1-4 substituents. In some cases, the heteroaryl ring is pyridyl, pyridazinyl, pyrimidinyl, or pyrazinyl; and the fused ring has 5 total atoms and 1 oxygen atom in the fused ring, 5 total atoms and 1 nitrogen atom in the fused ring, 6 total atoms and 1 nitrogen or oxygen atom in the ring, or 6 total atoms, 1 oxygen atom, and 1 nitrogen atom in the fused ring. In some cases, the bicyclic ring is unsubstituted. In some cases, the bicyclic ring is substituted with halo, CN, C₁₋₆ alkyl, C₁₋₆ haloalkyl, C₀₋₆ alkylene-OH, or C₀₋₆ alkylene-C₁₋₃ alkoxy, or any combination of the foregoing. In some cases, each substituent of the bicyclic ring independently is Br, Cl, F, CN, CH₃, CH₂CH₃, CH₂CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂), OH, CH₂OH, CH₂CH₂OH, CH(CH₃)CH₂OH, C(CH₃)₂OH, C(CH₃)₂CH₂OH, CH₂C(CH₃)₂OH, OCH₃, CH₂OCH₃, CH₂CH₂OCH₃, CH₂CH₂CH₂OCH₃, CH(CH)CH₂OCH₃, C(CH₃)₂CH₂OCH₃, CH₂CH(CH₃)OCH₃, or CH₂C(CH₃)₂OCH₃. In some cases, the bicyclic ring is substituted with Cl, Br, F, CH₃, OH, CH₂OH, OCH₃, or CH₂OCH₃. In some cases, Z is

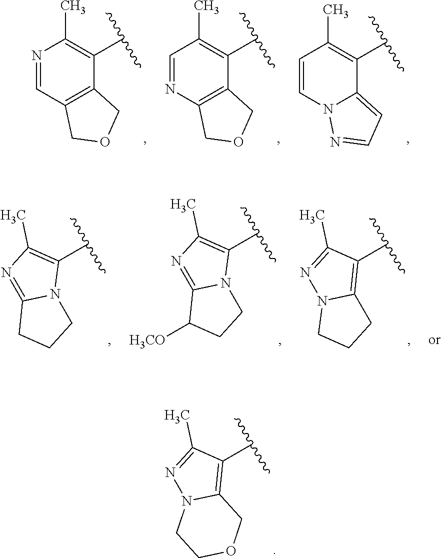

In some cases, the disclosure provides a compound of Formula (I):

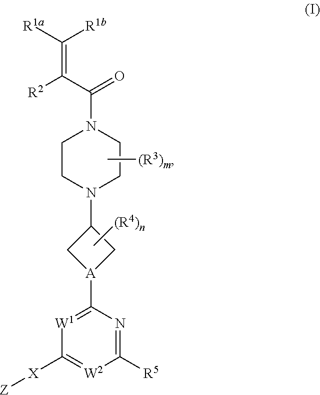

or a pharmaceutically acceptable salt thereof,
wherein:
m is 0, 1, 2, 3, or 4;
n is 0, 1, or 2;
A is N, CH, C-halo, C—CN, C—C₁₋₃ alkyl, C—C₁₋₃ haloalkyl, C—C₀₋₃ alkyleneOH, or C—C₁₋₃ alkylene-C₁₋₄ alkoxy;
each of W¹ and W² independently is N, CH, C-halo, C—CN, C—C₁₋₃ alkyl, C—C₂₋₃ alkenyl, C—C₂₋₃ alkynyl, C—C₁₋₃ haloalkyl, C—C₀₋₃ alkyleneOH, or C—C₀₋₃ alkylene-C₁₋₄ alkoxy, wherein each of the alkenyl and alkynyl is optionally substituted with 1-3 substituents and each substituent independently is halo, C₁₋₃ haloalkyl, C₀₋₃ alkyleneOH, or C₀₋₃ alkyleneC₁₋₄ alkoxy;
X is heterocycloalkyl or heterocycloalkenyl, each having 4-7 total ring atoms and 1-3 heteroatoms selected from N, O, and S, wherein each of the heterocycloalkyl and heterocycloalkenyl is optionally substituted with 1-3 substituents, and each substituent independently is halo, C₁₋₃ alkyl, C₁₋₃ haloalkyl, C₀₋₂ alkyleneOH, C₀₋₂ alkyleneC₁₋₃ alkoxy, or C₀₋₂ alkyleneCN;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S.
   wherein each of the phenyl, heteroaryl, and bicyclic rings is optionally substituted with 1-4 substituents, and each substituent independently is halo, CN, C₁₋₆ alkyl, C₁₋₆ haloalkyl, C₂₋₆ alkenyl, C₂₋₆ haloalkenyl, C₀₋₆ alkylene-OH, C₀₋₆ alkylene-C₁₋₃ alkoxy, C₀₋₆ alkylene-N(R^{N1})₂, C₀₋₂ alkylene-cycloalkyl having 3-6 total ring atoms, C₀₋₂ alkylene-heterocycloalkyl having 3-6 total ring atoms and 1-3 heteroatoms selected from N, O, and S, or C₀₋₂ alkylene-phenyl;
   wherein each of the C₁₋₆ alkyl, C₂₋₆ alkenyl, C₀₋₆ alkylene-C₀₋₆ alkoxy, cycloalkyl, heterocycloalkyl, and phenyl substituents is optionally substituted with 1-3 further substituents, and each further substituent independently is D, halo, C₁₋₃ alkyl, C₁₋₃ haloalkyl, C₁₋₂ alkyleneOH, C₁₋₂ alkylene-C₁₋₂ alkoxy, C₁₋₃ deuterated alkoxy, N(R^{N1})₂, (C═O)C₁₋₃ alkyl, cycloalkyl having 3-5 total ring atoms, heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S; or two adjacent further substituents, together with the atoms to which they are attached, form fused-cycloalkyl having 3-5 total ring atoms or fused-heterocycloalkyl having 3-5 total ring atoms and 1 or 2 heteroatoms selected from N, O, and S;
   wherein each of the foregoing cycloalkyl and heterocycloalkyl further substituents is optionally substituted with 1 or 2 substituents, and each substituent independently is halo or C₁₋₃ alkyl;
each of R^{1a}, R^{1b}, and R² independently is H, D, halo, C₁₋₄ alkyl, C₁₋₄ haloalkyl, C₁₋₂ alkylene-OH, C₀₋₂ alkylene-C₁₋₄ alkoxy, C₀₋₂ alkylene-C₁₋₄ haloalkoxy, C₀₋₂ alkylene-CN, C₀₋₂ alkylene-N(R^{N1})₂, C₁₋₂ alkylene-heterocycloalkyl having 3-6 total ring atoms and 1 or 2 heteroatoms selected from N, O and S or R^{1b} and R², together with the carbon atoms to which they are attached, form

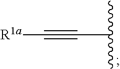

each R³ independently is C₁₋₃ alkyl, C₁₋₃ haloalkyl,

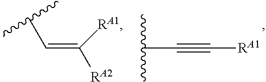

C₀₋₃ alkyleneCN, C₀₋₃ alkyleneOH, C₀₋₃ alkylene-C₁₋₃ alkoxy, oxo, spiro-cycloalkyl having 3-7 total ring atoms, spiro-cycloalkenyl having 4-7 total ring atoms, spiro-heterocycloalkyl having 3-7 total ring atoms and 1 or 2 heteroatoms selected from N, O and S, spiro-heterocycloalkenyl having 4-7 total ring atoms and 1 or 2 heteroatoms selected from N, O and S, or two adjacent R³, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1 or 2 heteroatoms selected from N, O, and S, or a fused heterocycloalkenyl ring having 4-7 total ring atoms and 1 or 2 heteroatoms selected from N, O, and S; or

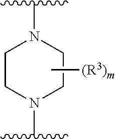

is deuterated:
each R⁴ independently is C₁₋₃ alkyl, C₁₋₃ haloalkyl, C₀₋₃ alkyleneCN, C₁₋₃ alkyleneOH, C₁₋₃ alkylene-C₁₋₃ alkoxy, oxo, spiro-cycloalkyl having 3-7 total ring atoms, or spiro-heterocycloalkyl having 4-7 total ring atoms and 1 or 2 heteroatoms selected from N, O and S;
R⁵ is C₁₋₃ haloalkyl, C₁₋₆ alkyl, C₂₋₄ alkenyl, C₂₋₄ alkynyl, halo, C₁₋₃ alkoxy, C₁₋₃ thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing independently is optionally substituted with 1-3 substituents, and each substituent independently is C₁₋₃ haloalkyl, C₀₋₆ alkylene-OH, C₀₋₆ alkylene-C₁₋₃ alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl;
each of R^{A1} and R^{A2} independently is H, C₁₋₃ alkyl, C₁₋₃ haloalkyl, or cycloalkyl having 3-5 total ring atoms; and each R^{N1} independently is H or C₁₋₄ alkyl;
   wherein the substituents are as defined herein.

In some cases,

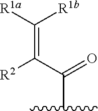

### of Formula (I) is

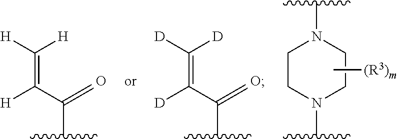

is

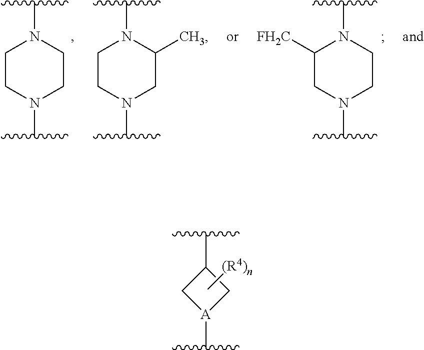

is

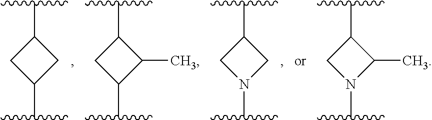

In some cases,

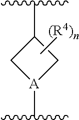

is

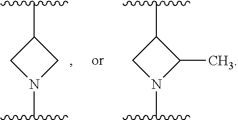 [SMILES: *C1CN(*)C1 ; *C1CN(*)C1C]

In some cases,

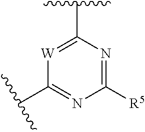

is

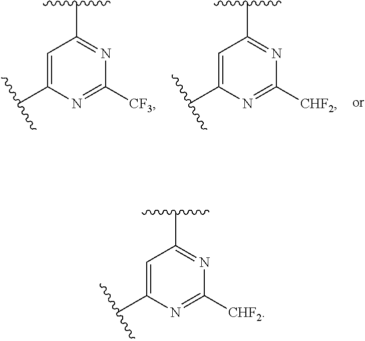 [SMILES: *c1cc(*)nc(C(F)(F)F)n1 ; *c1cc(*)nc(C(F)F)n1 ; "or" ; *c1cc(*)nc(C(F)F)n1]

In some cases, X is

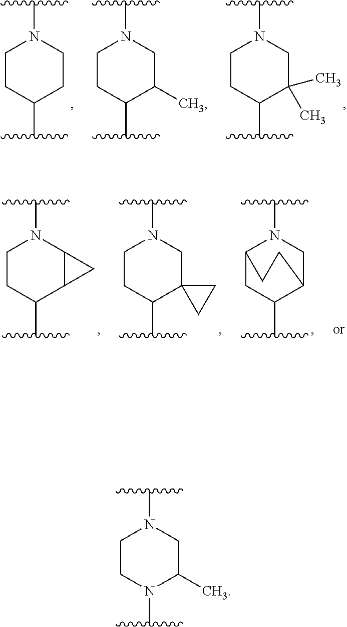

In some cases, Z is

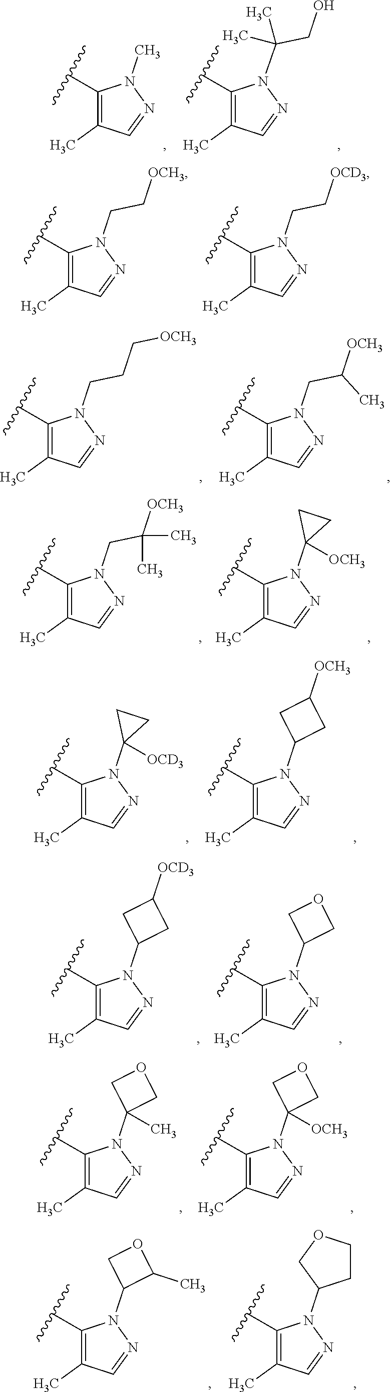

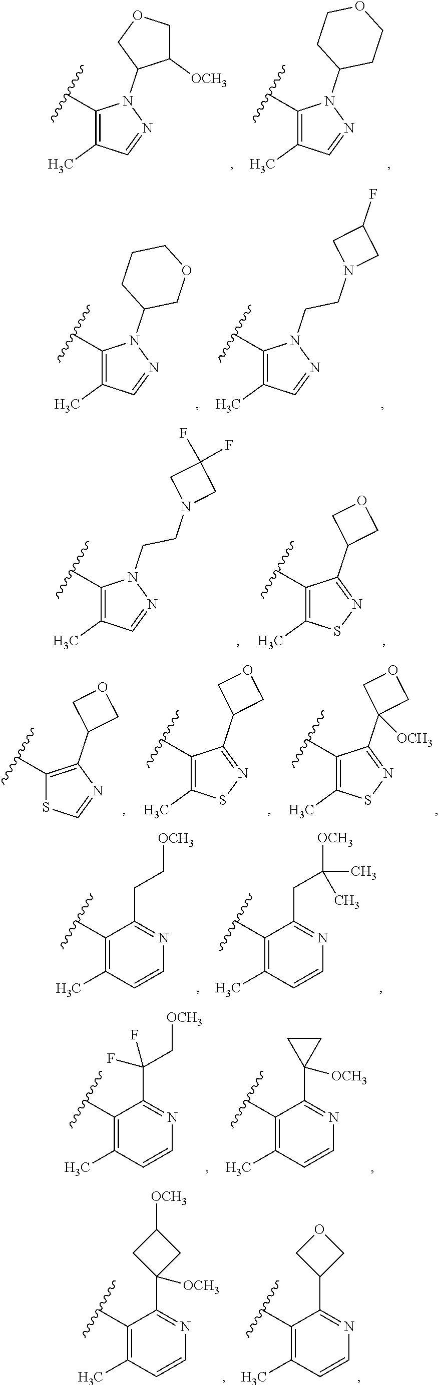

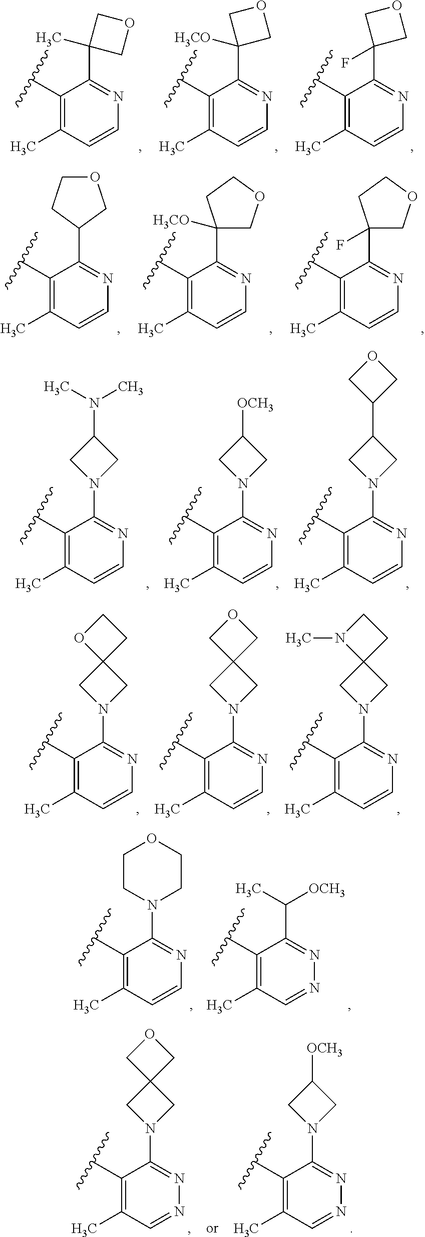

In some cases, Z is

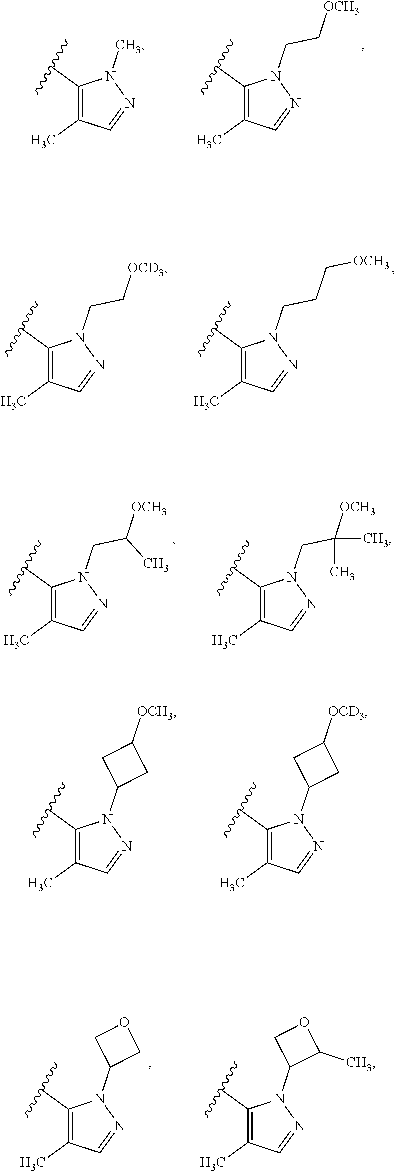

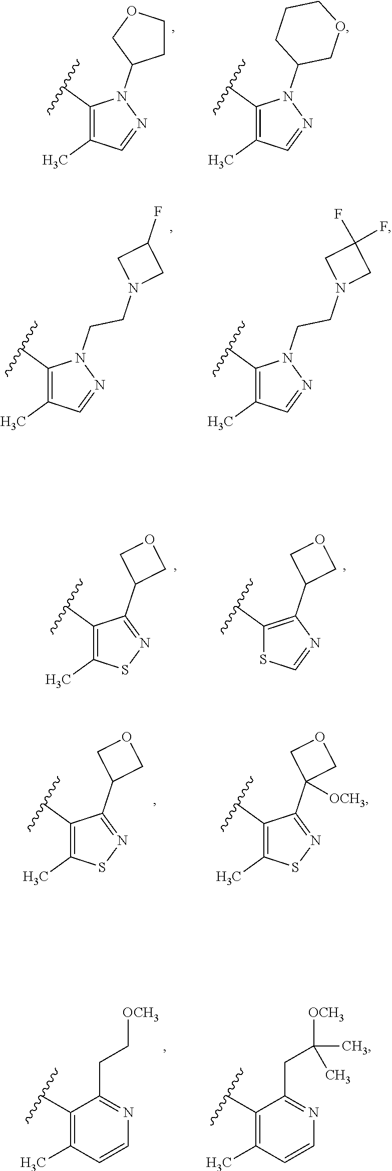

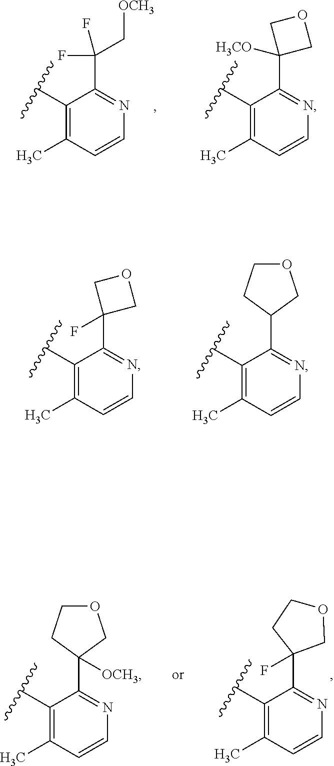 [SMILES: *c1c(C)ccnc1C(F)(F)COC ; *c1c(C)ccnc1C1(OC)COC1 ; *c1c(C)ccnc1C1(F)COC1 ; *c1c(C)ccnc1C1CCOC1 ; *c1c(C)ccnc1C1(OC)CCOC1 ; *c1c(C)ccnc1C1(F)CCOC1]

In some cases, Z is

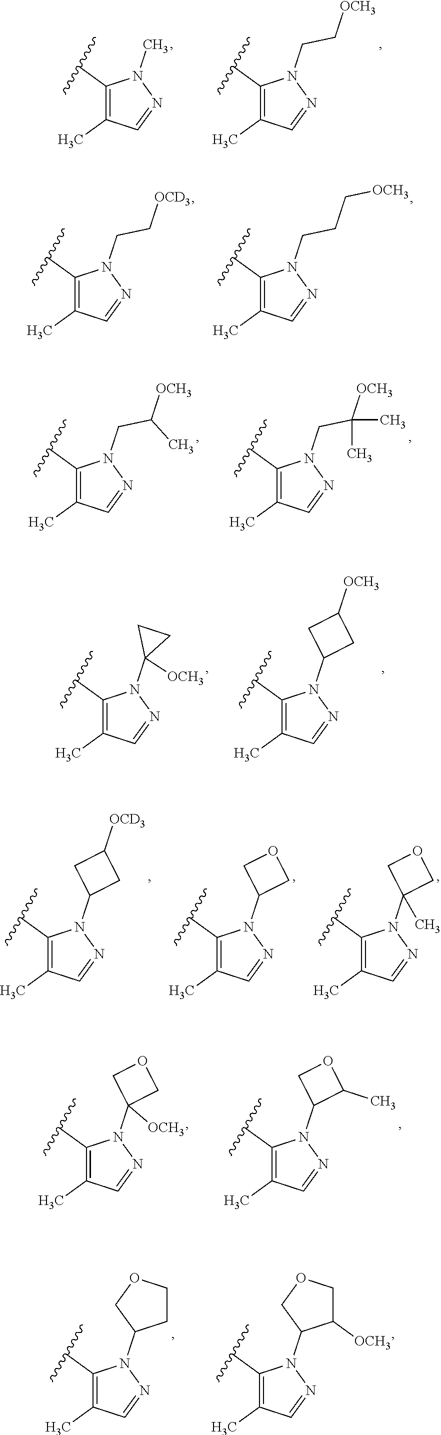

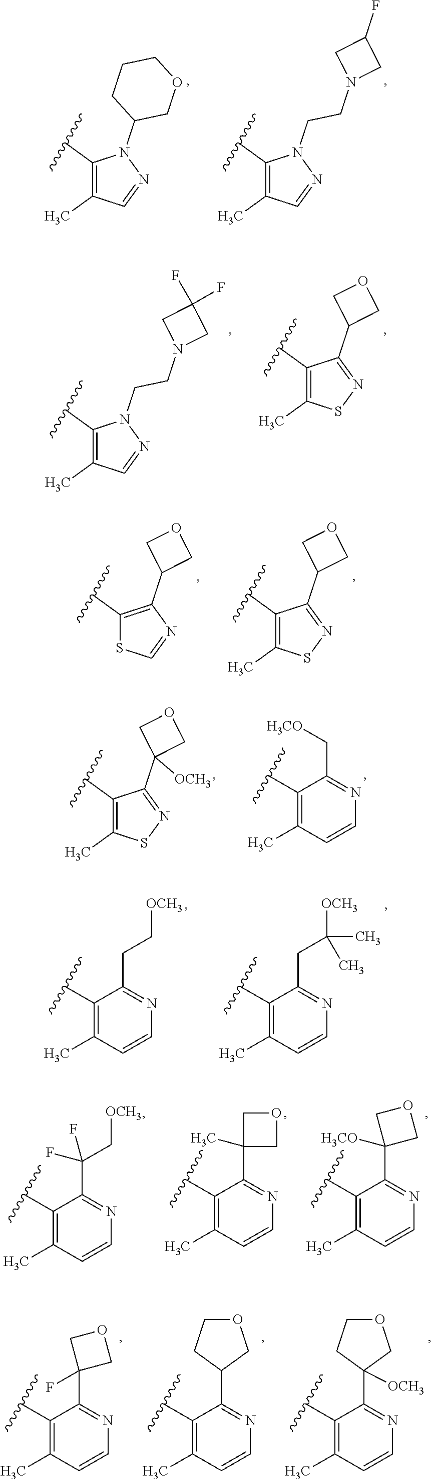

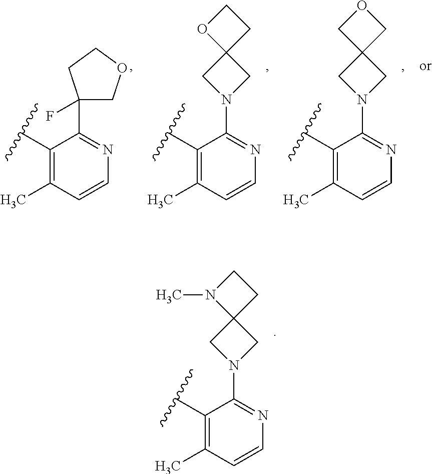 [SMILES: *c1c(C)ccnc1C1(F)CCOC1 ; *c1c(C)ccnc1N1CC2(CCO2)C1 ; *c1c(C)ccnc1N1CC2(COC2)C1 ; *c1c(C)ccnc1N1CC2(CCN2C)C1]

In some cases, Z is

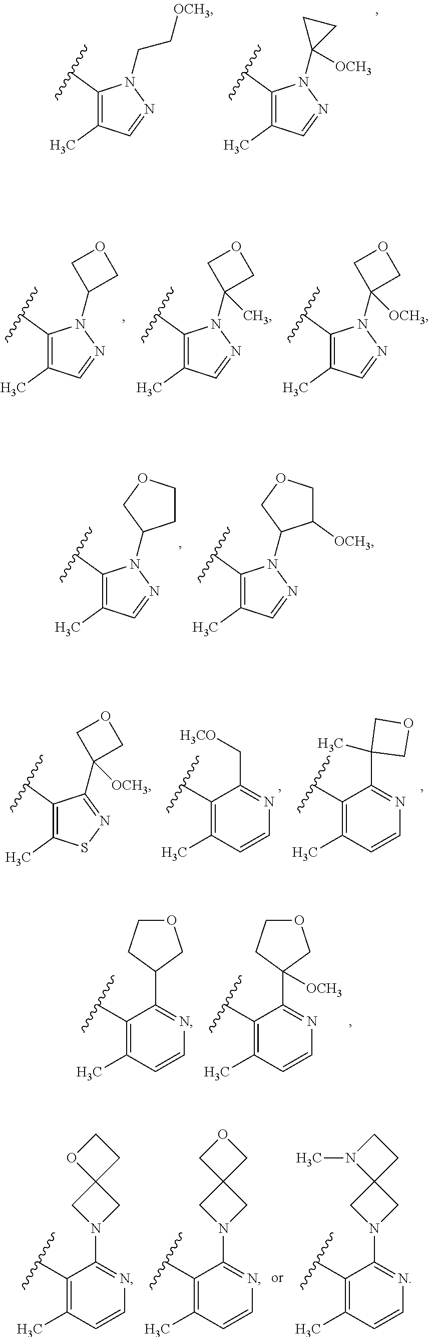

In some cases, Z is

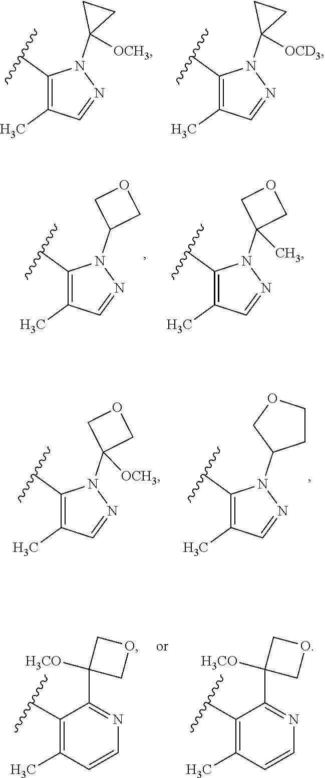

In some cases,

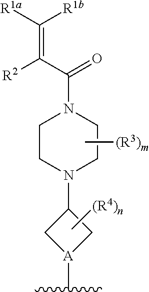

### of Formula (I) is

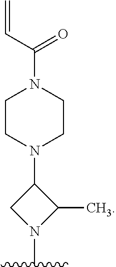 [SMILES: *N1CC(N2CCN(C(=O)C=C)CC2)C1C]

In some cases,

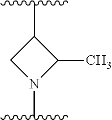 [SMILES: *C1CN(*)C1C]

exhibits the following stereochemical configuration:

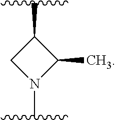 [SMILES: *[C@@H]1CN(*)[C@@H]1C]

In some cases,

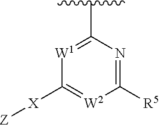

### of Formula (I) is

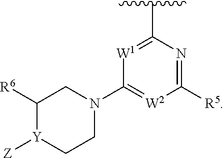

In some cases,

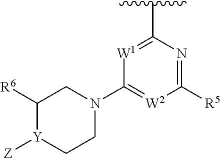

exhibits the following stereochemical configuration

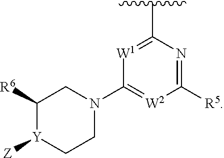

In some cases,

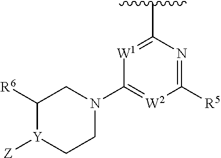

exhibits the following stereochemical configuration:

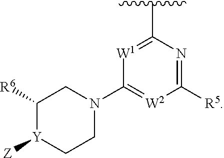

In some cases, the disclosure provides compounds of Formula (I′):

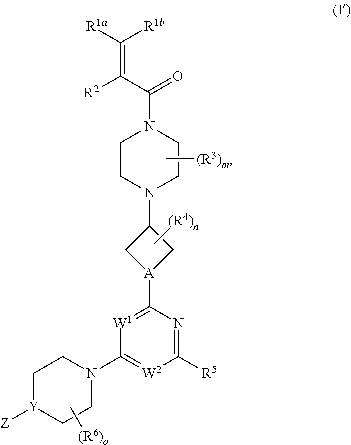

and pharmaceutically acceptable salts thereof, wherein the substituents are as previously described herein.

It is understood that selections of values of each variable are those that result in the formation of stable or chemically feasible compounds.

Specific compounds contemplated include compounds in the following Tables.

The compound of Formula (I) can be a compound as listed in Table A, or a pharmaceutically acceptable salt thereof.

TABLE A

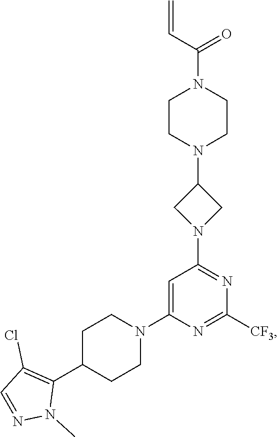 [SMILES: C=CC(=O)N1CCN(C2CN(c3cc(N4CCC(c5c(Cl)cnn5C)CC4)nc(C(F)(F)F)n3)C2)CC1]

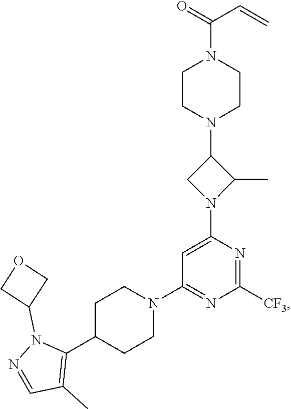 [SMILES: C=CC(=O)N1CCN(C2CN(c3cc(N4CCC(c5c(C)cnn5C5COC5)CC4)nc(C(F)(F)F)n3)C2C)CC1]

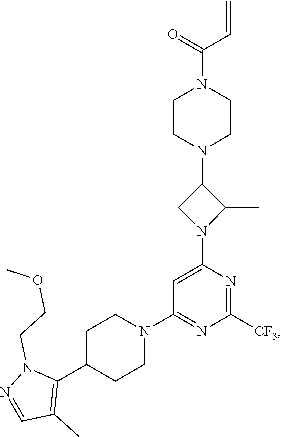 [SMILES: C=CC(=O)N1CCN(C2CN(c3cc(N4CCC(c5c(C)cnn5CCOC)CC4)nc(C(F)(F)F)n3)C2C)CC1]

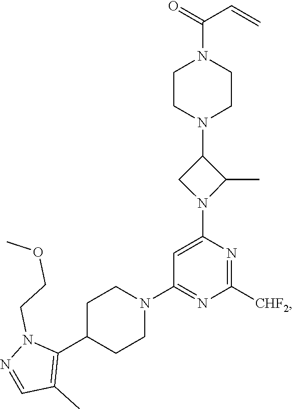 [SMILES: C=CC(=O)N1CCN(C2CN(c3cc(N4CCC(c5c(C)cnn5CCOC)CC4)nc(C(F)F)n3)C2C)CC1]

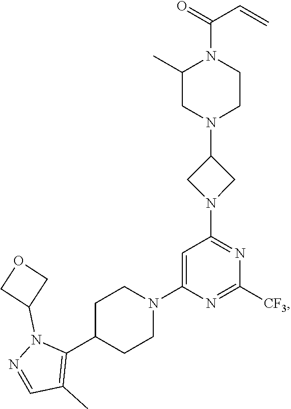 [SMILES: C=CC(=O)N1CCN(C2CN(c3cc(N4CCC(c5c(C)cnn5C5COC5)CC4)nc(C(F)(F)F)n3)C2)CC1C]

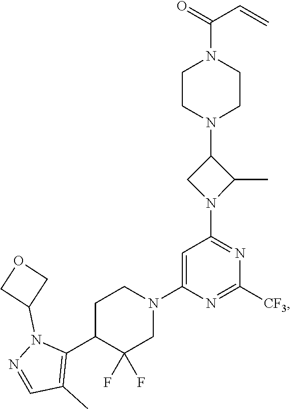 [SMILES: C=CC(=O)N1CCN(C2CN(c3cc(N4CCC(c5c(C)cnn5C5COC5)C(F)(F)C4)nc(C(F)(F)F)n3)C2C)CC1]

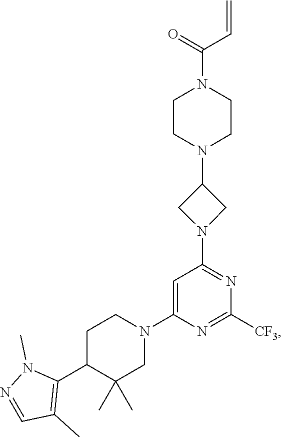 [SMILES: C=CC(=O)N1CCN(C2CN(c3cc(N4CCC(c5c(C)cnn5C)C(C)(C)C4)nc(C(F)(F)F)n3)C2)CC1]

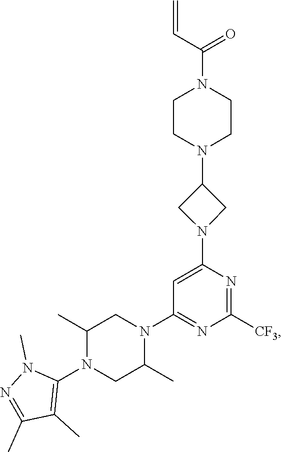 [SMILES: C=CC(=O)N1CCN(C2CN(c3cc(N4CC(C)N(c5c(C)c(C)nn5C)CC4C)nc(C(F)(F)F)n3)C2)CC1]

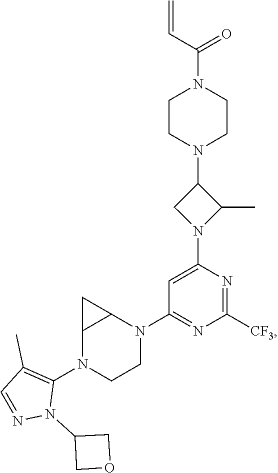 [SMILES: C=CC(=O)N1CCN(C2CN(c3cc(N4CCN(c5c(C)cnn5C5COC5)C5CC54)nc(C(F)(F)F)n3)C2C)CC1]

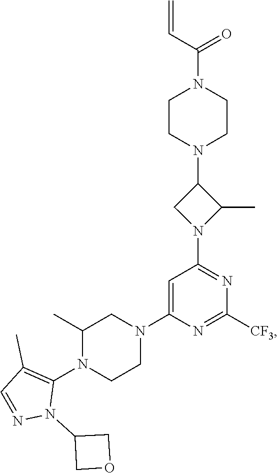 [SMILES: C=CC(=O)N1CCN(C2CN(c3cc(N4CCN(c5c(C)cnn5C5COC5)C(C)C4)nc(C(F)(F)F)n3)C2C)CC1]

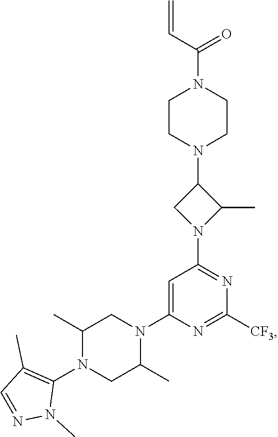 [SMILES: C=CC(=O)N1CCN(C2CN(c3cc(N4CC(C)N(c5c(C)cnn5C)CC4C)nc(C(F)(F)F)n3)C2C)CC1]

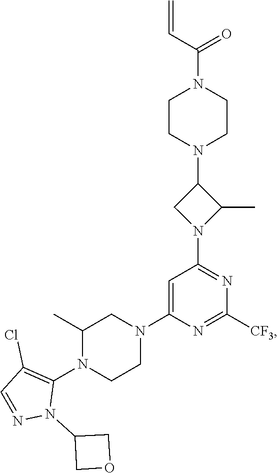 [SMILES: C=CC(=O)N1CCN(C2CN(c3cc(N4CCN(c5c(Cl)cnn5C5COC5)C(C)C4)nc(C(F)(F)F)n3)C2C)CC1]

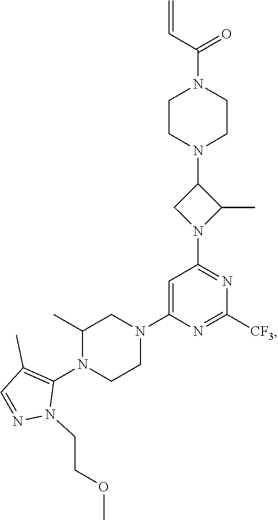 [SMILES: C=CC(=O)N1CCN(C2CN(c3cc(N4CCN(c5c(C)cnn5CCOC)C(C)C4)nc(C(F)(F)F)n3)C2C)CC1]

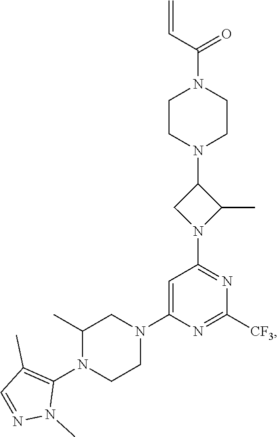 [SMILES: C=CC(=O)N1CCN(C2CN(c3cc(N4CCN(c5c(C)cnn5C)C(C)C4)nc(C(F)(F)F)n3)C2C)CC1]

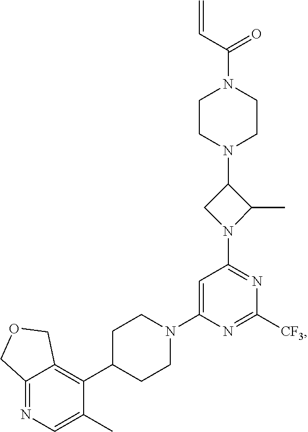 [SMILES: C=CC(=O)N1CCN(C2CN(c3cc(N4CCC(c5c(C)cnc6c5COC6)CC4)nc(C(F)(F)F)n3)C2C)CC1]

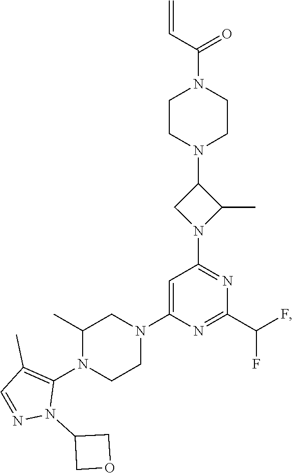 [SMILES: C=CC(=O)N1CCN(C2CN(c3cc(N4CCN(c5c(C)cnn5C5COC5)C(C)C4)nc(C(F)F)n3)C2C)CC1]

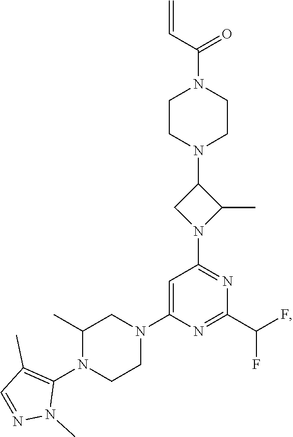 [SMILES: C=CC(=O)N1CCN(C2CN(c3cc(N4CCN(c5c(C)cnn5C)C(C)C4)nc(C(F)F)n3)C2C)CC1]

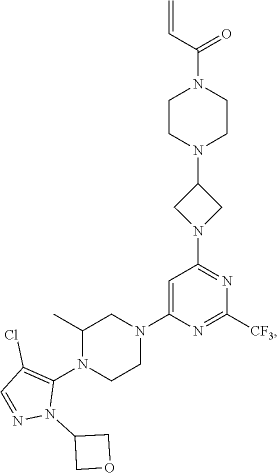 [SMILES: C=CC(=O)N1CCN(C2CN(c3cc(N4CCN(c5c(Cl)cnn5C5COC5)C(C)C4)nc(C(F)(F)F)n3)C2)CC1]

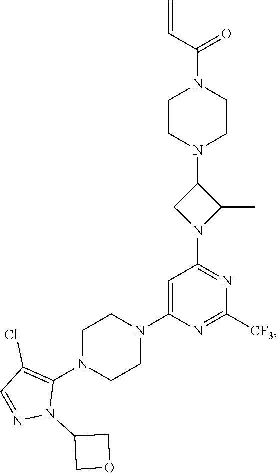 [SMILES: C=CC(=O)N1CCN(C2CN(c3cc(N4CCN(c5c(Cl)cnn5C5COC5)CC4)nc(C(F)(F)F)n3)C2C)CC1]

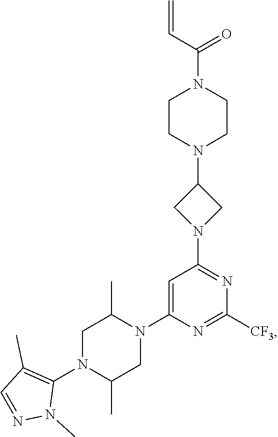 [SMILES: C=CC(=O)N1CCN(C2CN(c3cc(N4CC(C)N(c5c(C)cnn5C)CC4C)nc(C(F)(F)F)n3)C2)CC1]

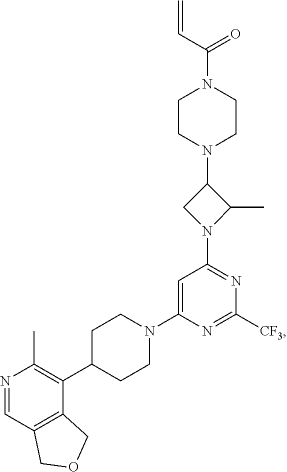 [SMILES: C=CC(=O)N1CCN(C2CN(c3cc(N4CCC(c5c(C)ncc6c5COC6)CC4)nc(C(F)(F)F)n3)C2C)CC1]

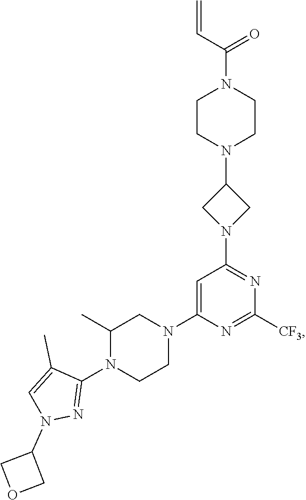 [SMILES: C=CC(=O)N1CCN(C2CN(c3cc(N4CCN(c5nn(C6COC6)cc5C)C(C)C4)nc(C(F)(F)F)n3)C2)CC1]

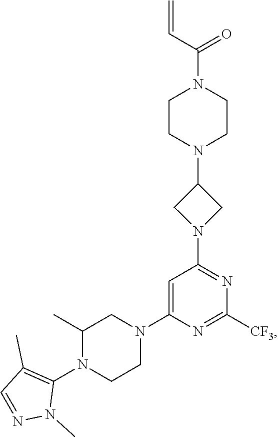 [SMILES: C=CC(=O)N1CCN(C2CN(c3cc(N4CCN(c5c(C)cnn5C)C(C)C4)nc(C(F)(F)F)n3)C2)CC1]

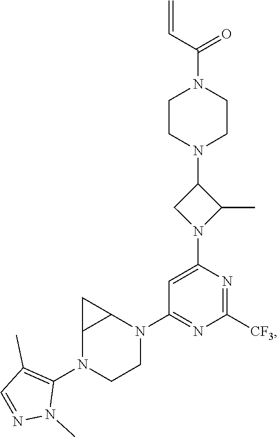 [SMILES: C=CC(=O)N1CCN(C2CN(c3cc(N4CCN(c5c(C)cnn5C)C5CC54)nc(C(F)(F)F)n3)C2C)CC1]

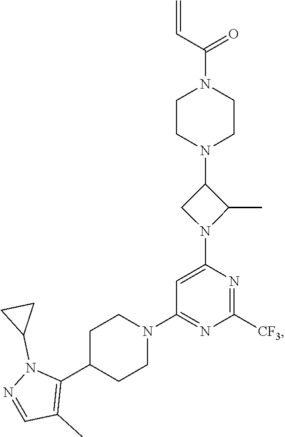 [SMILES: C=CC(=O)N1CCN(C2CN(c3cc(N4CCC(c5c(C)cnn5C5CC5)CC4)nc(C(F)(F)F)n3)C2C)CC1]

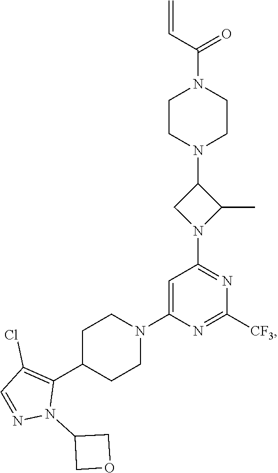 [SMILES: C=CC(=O)N1CCN(C2CN(c3cc(N4CCC(c5c(Cl)cnn5C5COC5)CC4)nc(C(F)(F)F)n3)C2C)CC1]

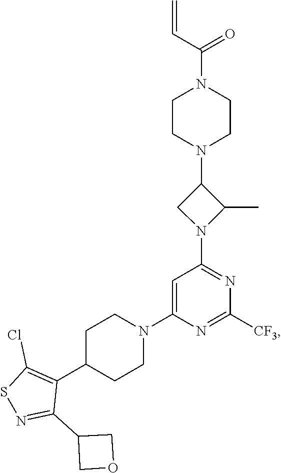 [SMILES: C=CC(=O)N1CCN(C2CN(c3cc(N4CCC(c5c(C6COC6)nsc5Cl)CC4)nc(C(F)(F)F)n3)C2C)CC1]

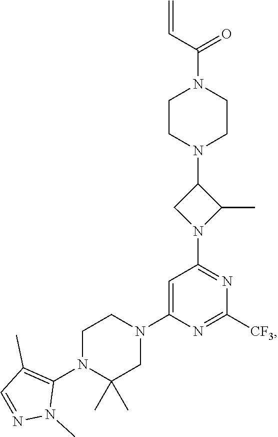 [SMILES: C=CC(=O)N1CCN(C2CN(c3cc(N4CCN(c5c(C)cnn5C)C(C)(C)C4)nc(C(F)(F)F)n3)C2C)CC1]

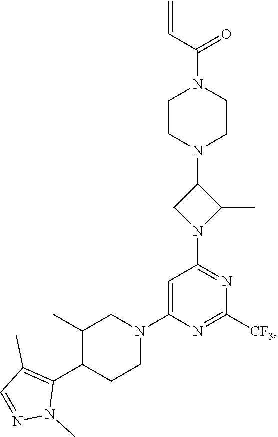 [SMILES: C=CC(=O)N1CCN(C2CN(c3cc(N4CCC(c5c(C)cnn5C)C(C)C4)nc(C(F)(F)F)n3)C2C)CC1]

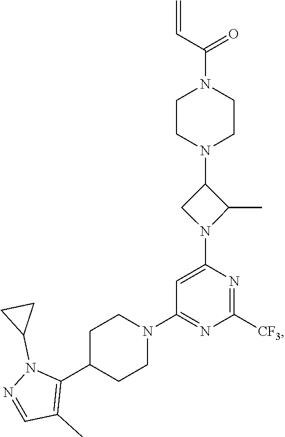 [SMILES: C=CC(=O)N1CCN(C2CN(c3cc(N4CCC(c5c(C)cnn5C5CC5)CC4)nc(C(F)(F)F)n3)C2C)CC1]

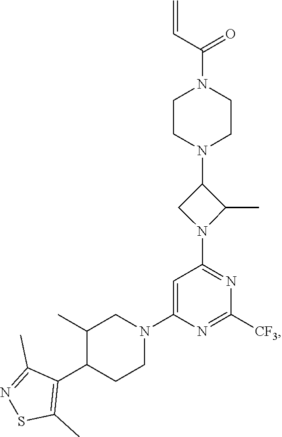 [SMILES: C=CC(=O)N1CCN(C2CN(c3cc(N4CCC(c5c(C)nsc5C)C(C)C4)nc(C(F)(F)F)n3)C2C)CC1]

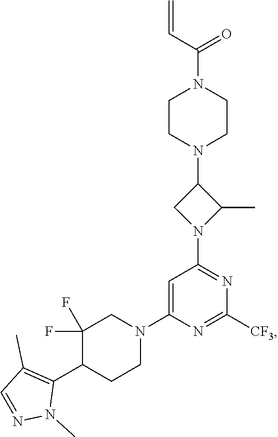 [SMILES: C=CC(=O)N1CCN(C2CN(c3cc(N4CCC(c5c(C)cnn5C)C(F)(F)C4)nc(C(F)(F)F)n3)C2C)CC1]

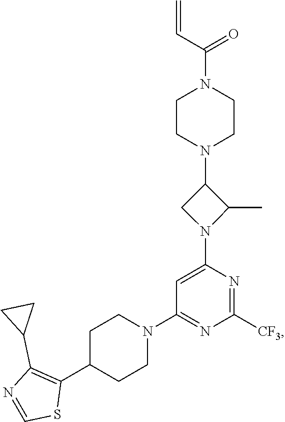 [SMILES: C=CC(=O)N1CCN(C2CN(c3cc(N4CCC(c5scnc5C5CC5)CC4)nc(C(F)(F)F)n3)C2C)CC1]

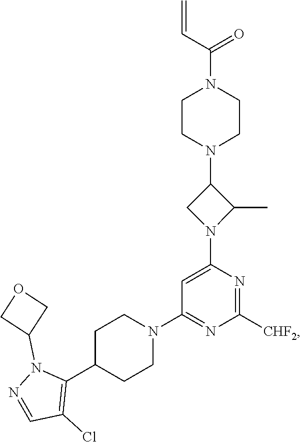 [SMILES: C=CC(=O)N1CCN(C2CN(c3cc(N4CCC(c5c(Cl)cnn5C5COC5)CC4)nc(C(F)F)n3)C2C)CC1]

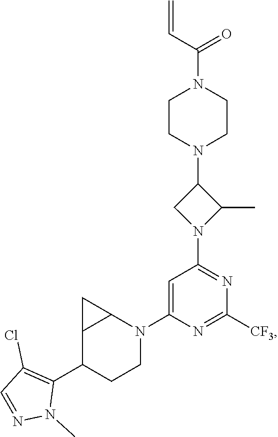 [SMILES: C=CC(=O)N1CCN(C2CN(c3cc(N4CCC(c5c(Cl)cnn5C)C5CC54)nc(C(F)(F)F)n3)C2C)CC1]

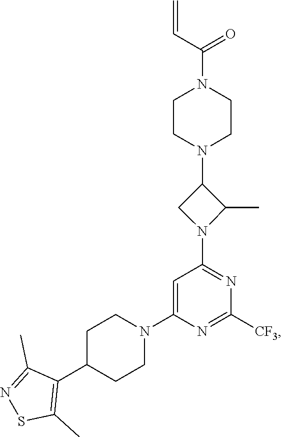 [SMILES: C=CC(=O)N1CCN(C2CN(c3cc(N4CCC(c5c(C)nsc5C)CC4)nc(C(F)(F)F)n3)C2C)CC1]

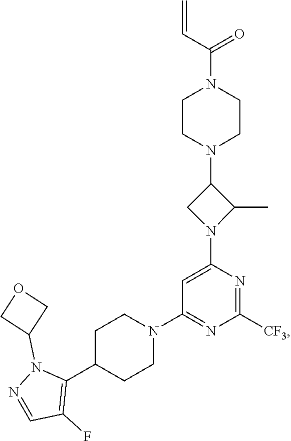 [SMILES: C=CC(=O)N1CCN(C2CN(c3cc(N4CCC(c5c(F)cnn5C5COC5)CC4)nc(C(F)(F)F)n3)C2C)CC1]

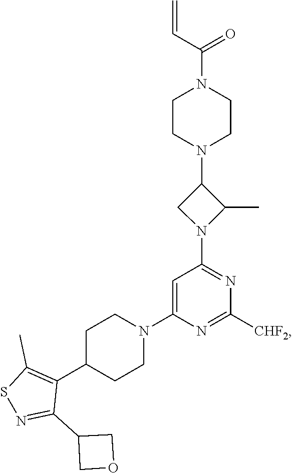 [SMILES: C=CC(=O)N1CCN(C2CN(c3cc(N4CCC(c5c(C6COC6)nsc5C)CC4)nc(C(F)F)n3)C2C)CC1]

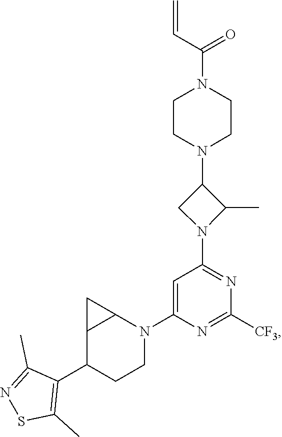 [SMILES: C=CC(=O)N1CCN(C2CN(c3cc(N4CCC(c5c(C)nsc5C)C5CC54)nc(C(F)(F)F)n3)C2C)CC1]

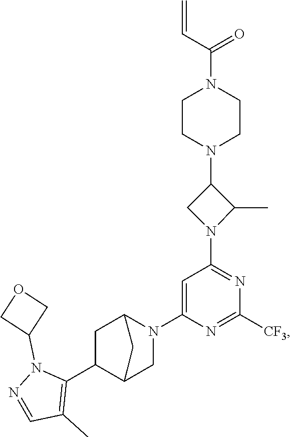 [SMILES: C=CC(=O)N1CCN(C2CN(c3cc(N4CC5CC4CC5c4c(C)cnn4C4COC4)nc(C(F)(F)F)n3)C2C)CC1]

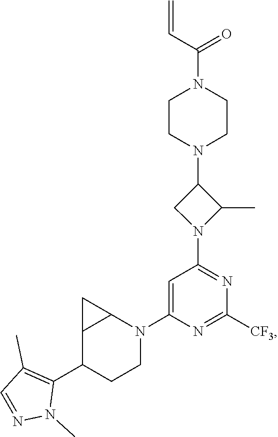 [SMILES: C=CC(=O)N1CCN(C2CN(c3cc(N4CCC(c5c(C)cnn5C)C5CC54)nc(C(F)(F)F)n3)C2C)CC1]

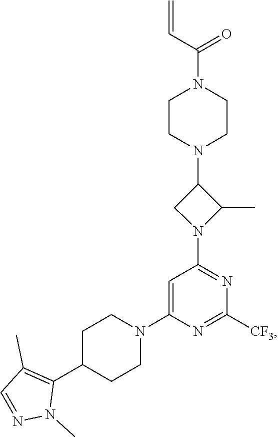 [SMILES: C=CC(=O)N1CCN(C2CN(c3cc(N4CCC(c5c(C)cnn5C)CC4)nc(C(F)(F)F)n3)C2C)CC1]

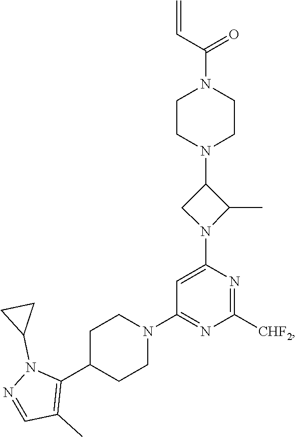 [SMILES: C=CC(=O)N1CCN(C2CN(c3cc(N4CCC(c5c(C)cnn5C5CC5)CC4)nc(C(F)F)n3)C2C)CC1]

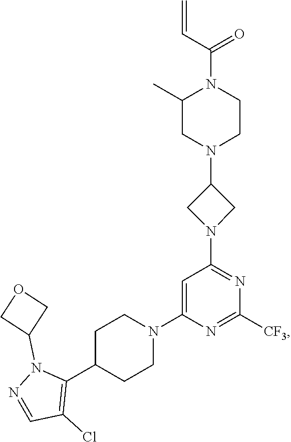 [SMILES: C=CC(=O)N1CCN(C2CN(c3cc(N4CCC(c5c(Cl)cnn5C5COC5)CC4)nc(C(F)(F)F)n3)C2)CC1C]

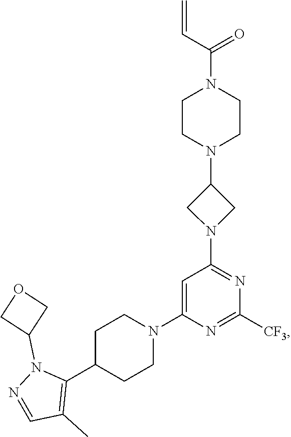 [SMILES: C=CC(=O)N1CCN(C2CN(c3cc(N4CCC(c5c(C)cnn5C5COC5)CC4)nc(C(F)(F)F)n3)C2)CC1]

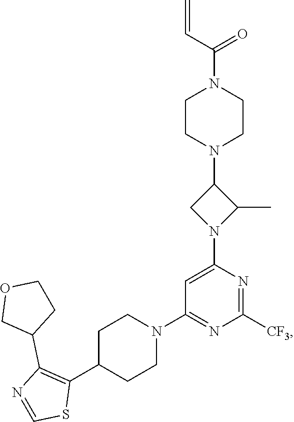 [SMILES: C=CC(=O)N1CCN(C2CN(c3cc(N4CCC(c5scnc5C5CCOC5)CC4)nc(C(F)(F)F)n3)C2C)CC1]

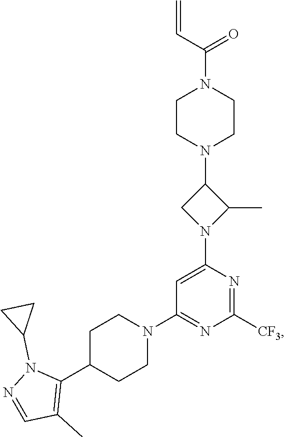 [SMILES: C=CC(=O)N1CCN(C2CN(c3cc(N4CCC(c5c(C)cnn5C5CC5)CC4)nc(C(F)(F)F)n3)C2C)CC1]

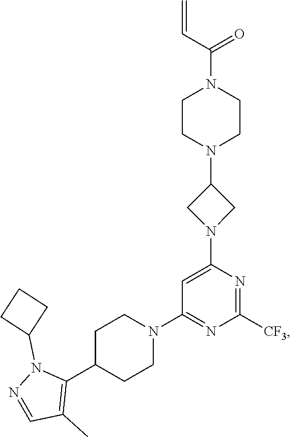 [SMILES: C=CC(=O)N1CCN(C2CN(c3cc(N4CCC(c5c(C)cnn5C5CCC5)CC4)nc(C(F)(F)F)n3)C2)CC1]

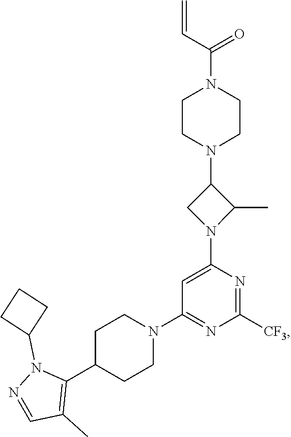 [SMILES: C=CC(=O)N1CCN(C2CN(c3cc(N4CCC(c5c(C)cnn5C5CCC5)CC4)nc(C(F)(F)F)n3)C2C)CC1]

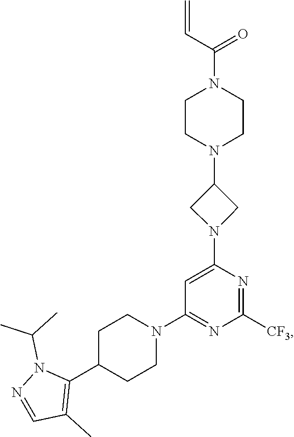 [SMILES: C=CC(=O)N1CCN(C2CN(c3cc(N4CCC(c5c(C)cnn5C(C)C)CC4)nc(C(F)(F)F)n3)C2)CC1]

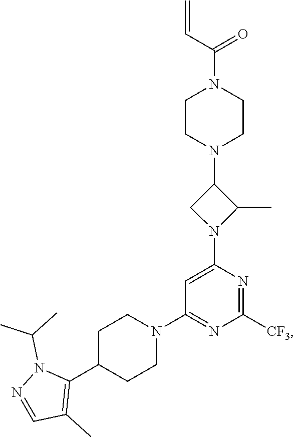 [SMILES: C=CC(=O)N1CCN(C2CN(c3cc(N4CCC(c5c(C)cnn5C(C)C)CC4)nc(C(F)(F)F)n3)C2C)CC1]

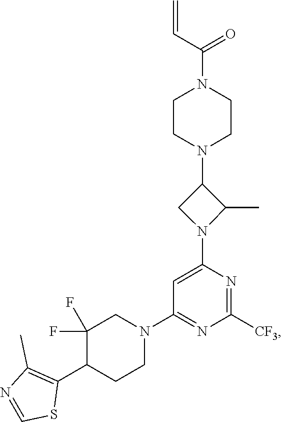 [SMILES: C=CC(=O)N1CCN(C2CN(c3cc(N4CCC(c5scnc5C)C(F)(F)C4)nc(C(F)(F)F)n3)C2C)CC1]

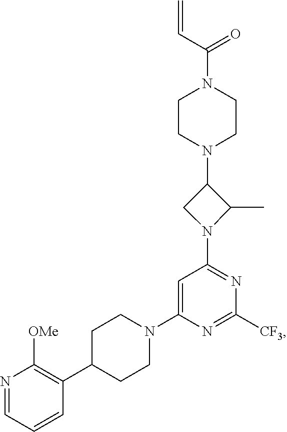 [SMILES: C=CC(=O)N1CCN(C2CN(c3cc(N4CCC(c5cccnc5OC)CC4)nc(C(F)(F)F)n3)C2C)CC1]

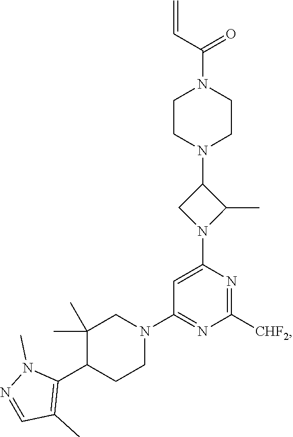 [SMILES: C=CC(=O)N1CCN(C2CN(c3cc(N4CCC(c5c(C)cnn5C)C(C)(C)C4)nc(C(F)F)n3)C2C)CC1]

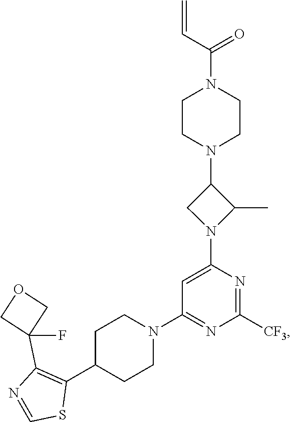 [SMILES: C=CC(=O)N1CCN(C2CN(c3cc(N4CCC(c5scnc5C5(F)COC5)CC4)nc(C(F)(F)F)n3)C2C)CC1]

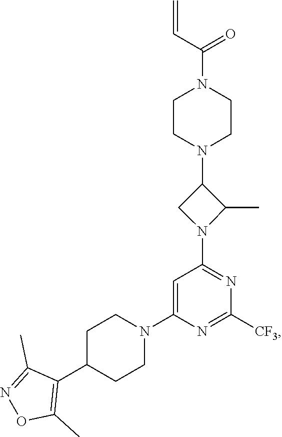 [SMILES: C=CC(=O)N1CCN(C2CN(c3cc(N4CCC(c5c(C)noc5C)CC4)nc(C(F)(F)F)n3)C2C)CC1]

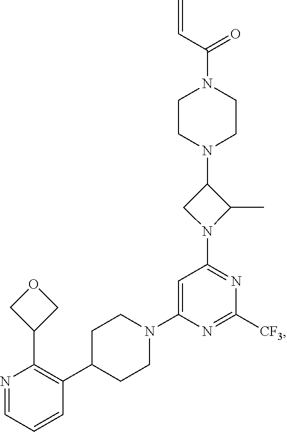 [SMILES: C=CC(=O)N1CCN(C2CN(c3cc(N4CCC(c5cccnc5C5COC5)CC4)nc(C(F)(F)F)n3)C2C)CC1]

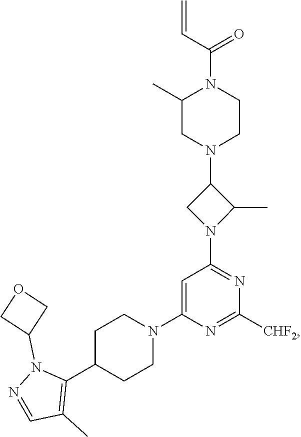 [SMILES: C=CC(=O)N1CCN(C2CN(c3cc(N4CCC(c5c(C)cnn5C5COC5)CC4)nc(C(F)F)n3)C2C)CC1C]

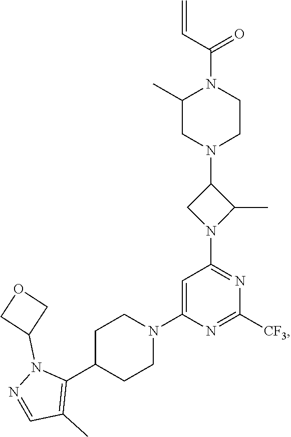 [SMILES: C=CC(=O)N1CCN(C2CN(c3cc(N4CCC(c5c(C)cnn5C5COC5)CC4)nc(C(F)(F)F)n3)C2C)CC1C]

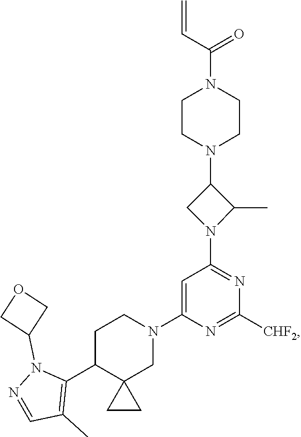 [SMILES: C=CC(=O)N1CCN(C2CN(c3cc(N4CCC(c5c(C)cnn5C5COC5)C5(CC5)C4)nc(C(F)F)n3)C2C)CC1]

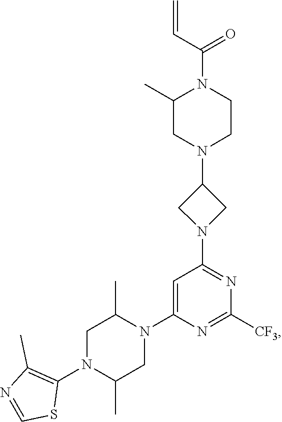 [SMILES: C=CC(=O)N1CCN(C2CN(c3cc(N4CC(C)N(c5scnc5C)CC4C)nc(C(F)(F)F)n3)C2)CC1C]

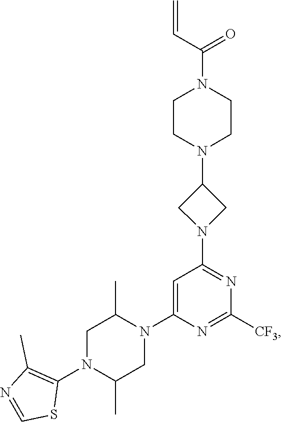 [SMILES: C=CC(=O)N1CCN(C2CN(c3cc(N4CC(C)N(c5scnc5C)CC4C)nc(C(F)(F)F)n3)C2)CC1]

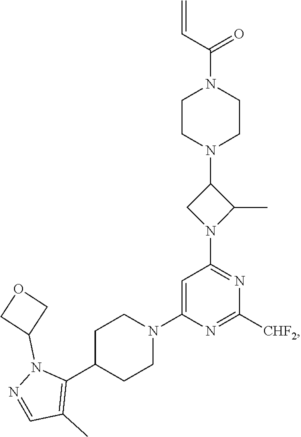 [SMILES: C=CC(=O)N1CCN(C2CN(c3cc(N4CCC(c5c(C)cnn5C5COC5)CC4)nc(C(F)F)n3)C2C)CC1]

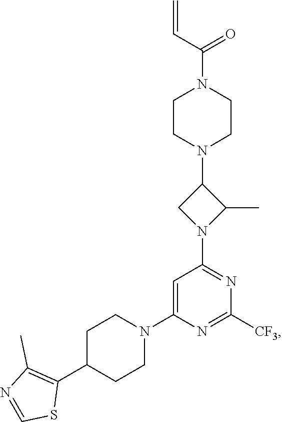 [SMILES: C=CC(=O)N1CCN(C2CN(c3cc(N4CCC(c5scnc5C)CC4)nc(C(F)(F)F)n3)C2C)CC1]

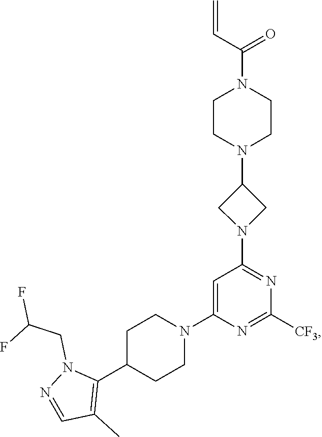 [SMILES: C=CC(=O)N1CCN(C2CN(c3cc(N4CCC(c5c(C)cnn5CC(F)F)CC4)nc(C(F)(F)F)n3)C2)CC1]

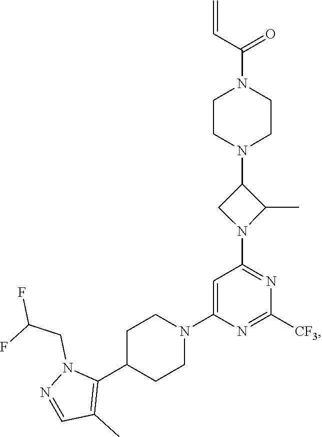 [SMILES: C=CC(=O)N1CCN(C2CN(c3cc(N4CCC(c5c(C)cnn5CC(F)F)CC4)nc(C(F)(F)F)n3)C2C)CC1]

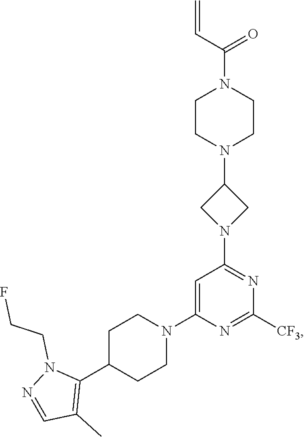 [SMILES: C=CC(=O)N1CCN(C2CN(c3cc(N4CCC(c5c(C)cnn5CCF)CC4)nc(C(F)(F)F)n3)C2)CC1]

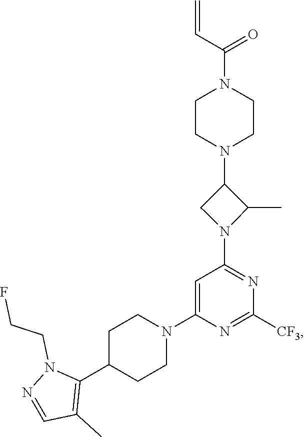 [SMILES: C=CC(=O)N1CCN(C2CN(c3cc(N4CCC(c5c(C)cnn5CCF)CC4)nc(C(F)(F)F)n3)C2C)CC1]

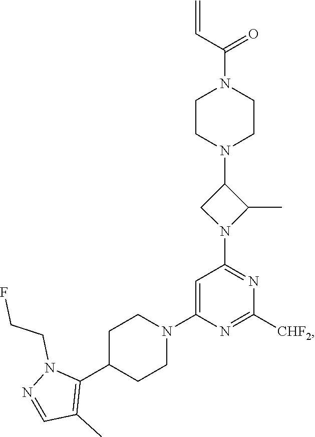 [SMILES: C=CC(=O)N1CCN(C2CN(c3cc(N4CCC(c5c(C)cnn5CCF)CC4)nc(C(F)F)n3)C2C)CC1]

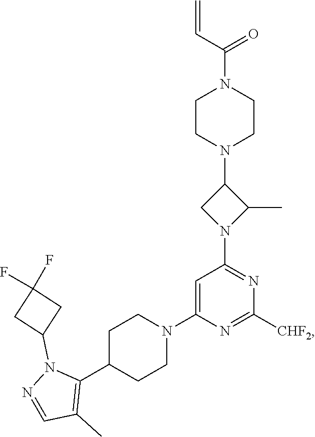 [SMILES: C=CC(=O)N1CCN(C2CN(c3cc(N4CCC(c5c(C)cnn5C5CC(F)(F)C5)CC4)nc(C(F)F)n3)C2C)CC1]

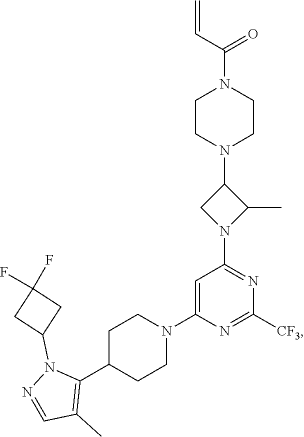 [SMILES: C=CC(=O)N1CCN(C2CN(c3cc(N4CCC(c5c(C)cnn5C5CC(F)(F)C5)CC4)nc(C(F)(F)F)n3)C2C)CC1]

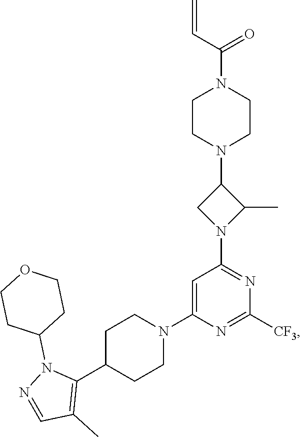 [SMILES: C=CC(=O)N1CCN(C2CN(c3cc(N4CCC(c5c(C)cnn5C5CCOCC5)CC4)nc(C(F)(F)F)n3)C2C)CC1]

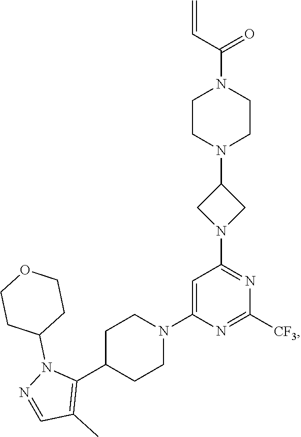 [SMILES: C=CC(=O)N1CCN(C2CN(c3cc(N4CCC(c5c(C)cnn5C5CCOCC5)CC4)nc(C(F)(F)F)n3)C2)CC1]

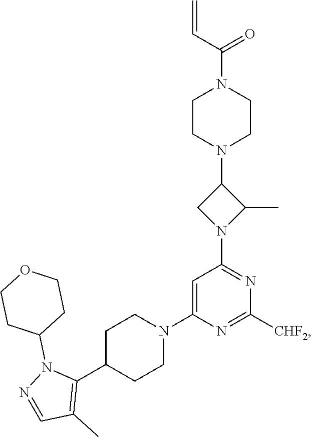 [SMILES: C=CC(=O)N1CCN(C2CN(c3cc(N4CCC(c5c(C)cnn5C5CCOCC5)CC4)nc(C(F)F)n3)C2C)CC1]

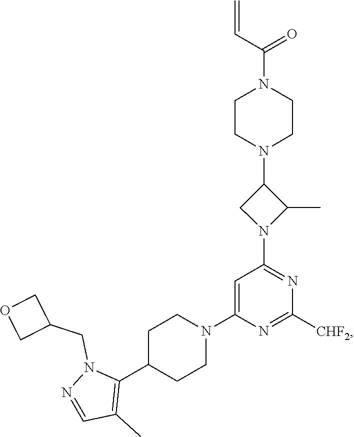 [SMILES: C=CC(=O)N1CCN(C2CN(c3cc(N4CCC(c5c(C)cnn5CC5COC5)CC4)nc(C(F)F)n3)C2C)CC1]

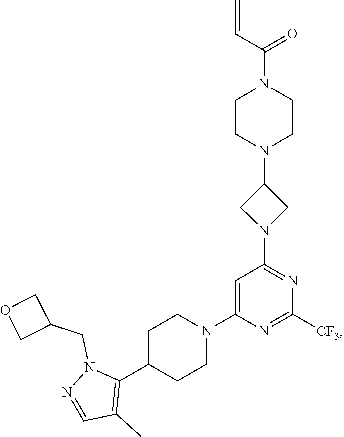 [SMILES: C=CC(=O)N1CCN(C2CN(c3cc(N4CCC(c5c(C)cnn5CC5COC5)CC4)nc(C(F)(F)F)n3)C2)CC1]

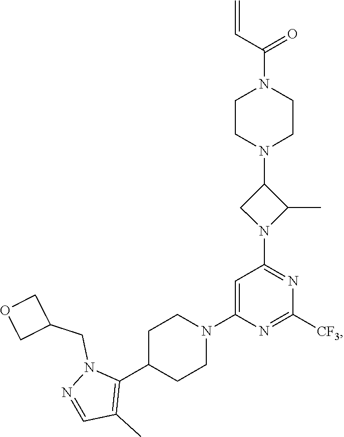 [SMILES: C=CC(=O)N1CCN(C2CN(c3cc(N4CCC(c5c(C)cnn5CC5COC5)CC4)nc(C(F)(F)F)n3)C2C)CC1]

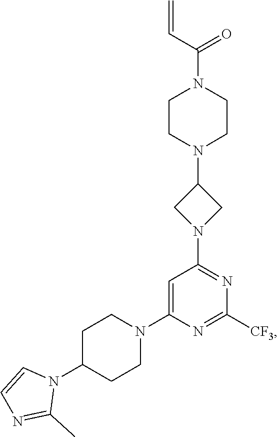 [SMILES: C=CC(=O)N1CCN(C2CN(c3cc(N4CCC(n5ccnc5C)CC4)nc(C(F)(F)F)n3)C2)CC1]

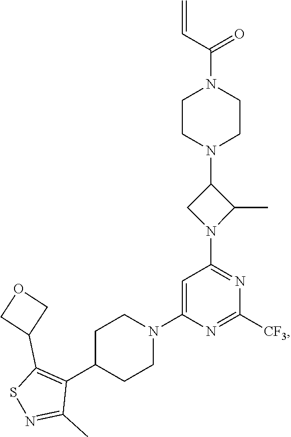 [SMILES: C=CC(=O)N1CCN(C2CN(c3cc(N4CCC(c5c(C)nsc5C5COC5)CC4)nc(C(F)(F)F)n3)C2C)CC1]

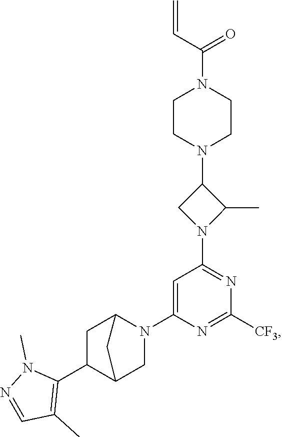 [SMILES: C=CC(=O)N1CCN(C2CN(c3cc(N4CC5CC4CC5c4c(C)cnn4C)nc(C(F)(F)F)n3)C2C)CC1]

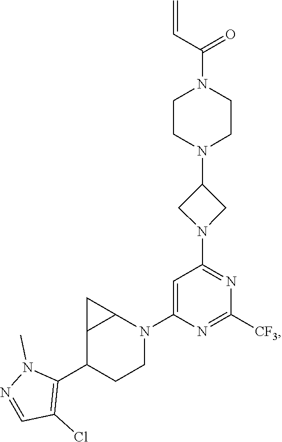 [SMILES: C=CC(=O)N1CCN(C2CN(c3cc(N4CCC(c5c(Cl)cnn5C)C5CC54)nc(C(F)(F)F)n3)C2)CC1]

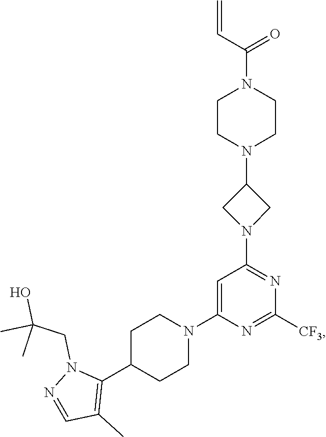 [SMILES: C=CC(=O)N1CCN(C2CN(c3cc(N4CCC(c5c(C)cnn5CC(C)(C)O)CC4)nc(C(F)(F)F)n3)C2)CC1]

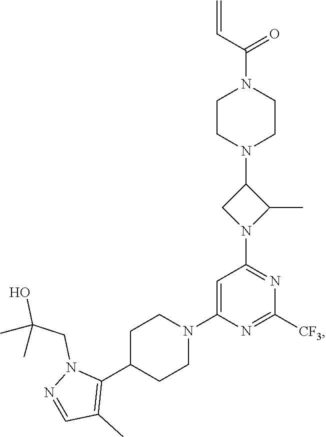 [SMILES: C=CC(=O)N1CCN(C2CN(c3cc(N4CCC(c5c(C)cnn5CC(C)(C)O)CC4)nc(C(F)(F)F)n3)C2C)CC1]

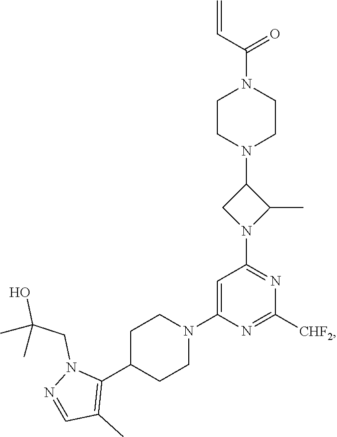 [SMILES: C=CC(=O)N1CCN(C2CN(c3cc(N4CCC(c5c(C)cnn5CC(C)(C)O)CC4)nc(C(F)F)n3)C2C)CC1]

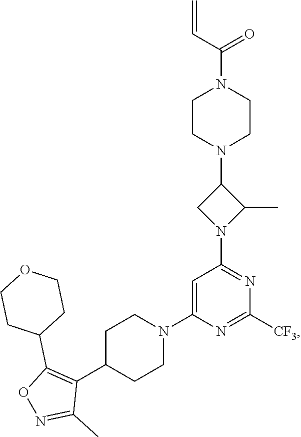 [SMILES: C=CC(=O)N1CCN(C2CN(c3cc(N4CCC(c5c(C)noc5C5CCOCC5)CC4)nc(C(F)(F)F)n3)C2C)CC1]

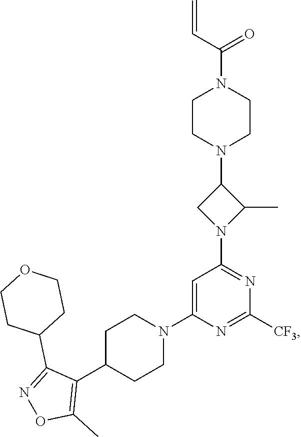 [SMILES: C=CC(=O)N1CCN(C2CN(c3cc(N4CCC(c5c(C6CCOCC6)noc5C)CC4)nc(C(F)(F)F)n3)C2C)CC1]

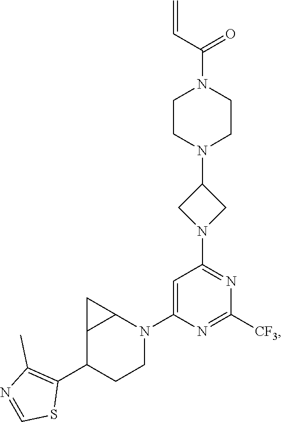 [SMILES: C=CC(=O)N1CCN(C2CN(c3cc(N4CCC(c5scnc5C)C5CC54)nc(C(F)(F)F)n3)C2)CC1]

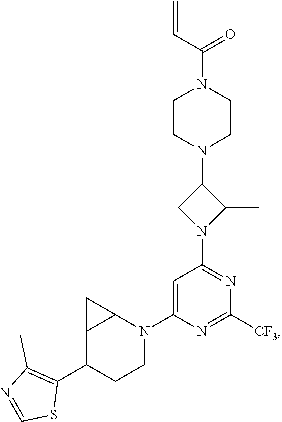 [SMILES: C=CC(=O)N1CCN(C2CN(c3cc(N4CCC(c5scnc5C)C5CC54)nc(C(F)(F)F)n3)C2C)CC1]

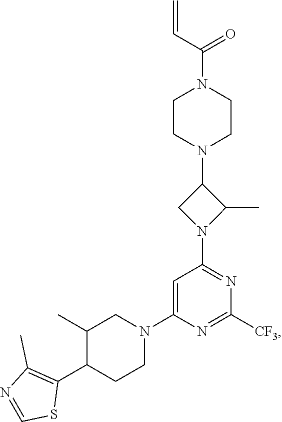 [SMILES: C=CC(=O)N1CCN(C2CN(c3cc(N4CCC(c5scnc5C)C(C)C4)nc(C(F)(F)F)n3)C2C)CC1]

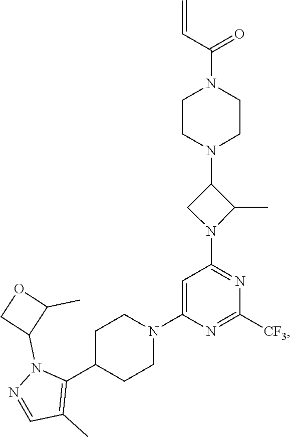 [SMILES: C=CC(=O)N1CCN(C2CN(c3cc(N4CCC(c5c(C)cnn5C5COC5C)CC4)nc(C(F)(F)F)n3)C2C)CC1]

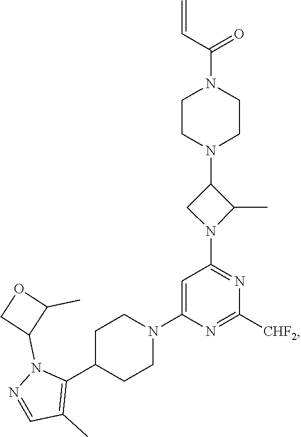 [SMILES: C=CC(=O)N1CCN(C2CN(c3cc(N4CCC(c5c(C)cnn5C5COC5C)CC4)nc(C(F)F)n3)C2C)CC1]

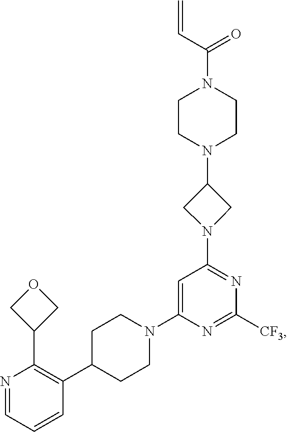 [SMILES: C=CC(=O)N1CCN(C2CN(c3cc(N4CCC(c5cccnc5C5COC5)CC4)nc(C(F)(F)F)n3)C2)CC1]

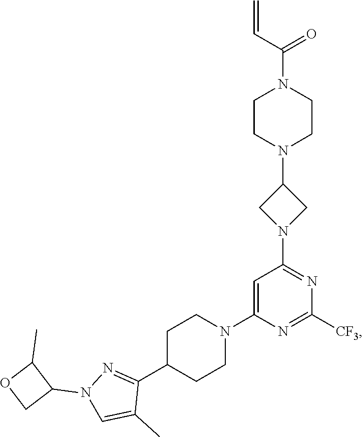 [SMILES: C=CC(=O)N1CCN(C2CN(c3cc(N4CCC(c5nn(C6COC6C)cc5C)CC4)nc(C(F)(F)F)n3)C2)CC1]

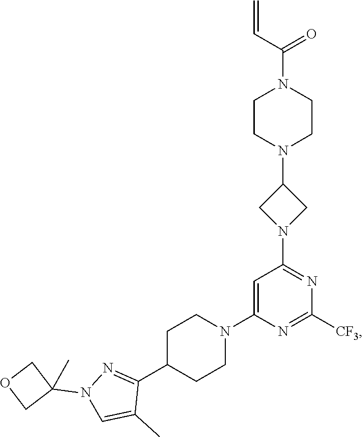 [SMILES: C=CC(=O)N1CCN(C2CN(c3cc(N4CCC(c5nn(C6(C)COC6)cc5C)CC4)nc(C(F)(F)F)n3)C2)CC1]

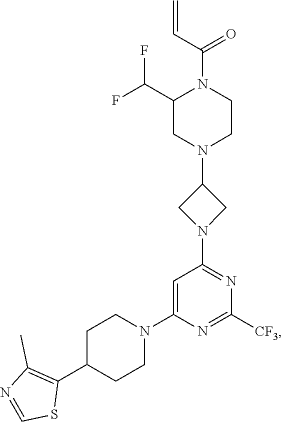 [SMILES: C=CC(=O)N1CCN(C2CN(c3cc(N4CCC(c5scnc5C)CC4)nc(C(F)(F)F)n3)C2)CC1C(F)F]

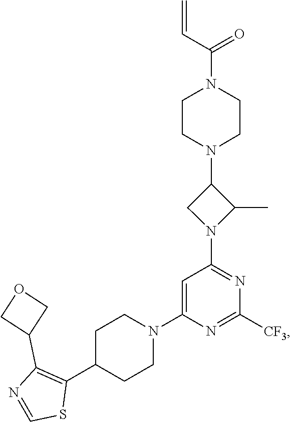 [SMILES: C=CC(=O)N1CCN(C2CN(c3cc(N4CCC(c5scnc5C5COC5)CC4)nc(C(F)(F)F)n3)C2C)CC1]

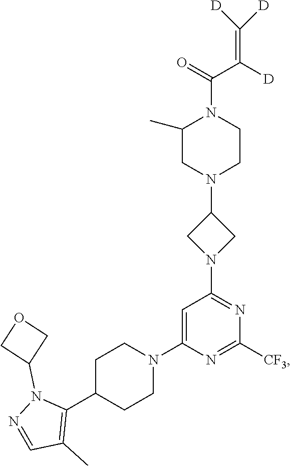 [SMILES: [2H]C([2H])=C([2H])C(=O)N1CCN(C2CN(c3cc(N4CCC(c5c(C)cnn5C5COC5)CC4)nc(C(F)(F)F)n3)C2)CC1C]

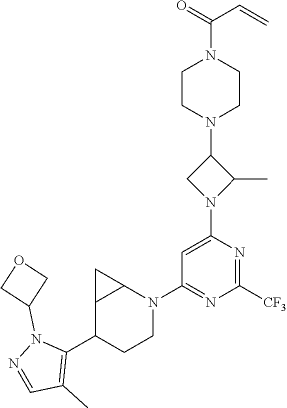 [SMILES: C=CC(=O)N1CCN(C2CN(c3cc(N4CCC(c5c(C)cnn5C5COC5)C5CC54)nc(C(F)(F)F)n3)C2C)CC1]

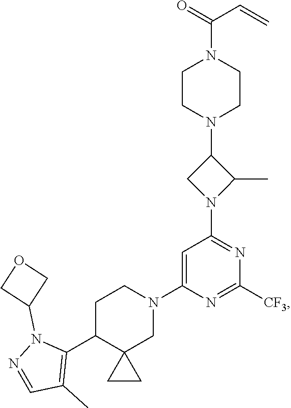 [SMILES: C=CC(=O)N1CCN(C2CN(c3cc(N4CCC(c5c(C)cnn5C5COC5)C5(CC5)C4)nc(C(F)(F)F)n3)C2C)CC1]

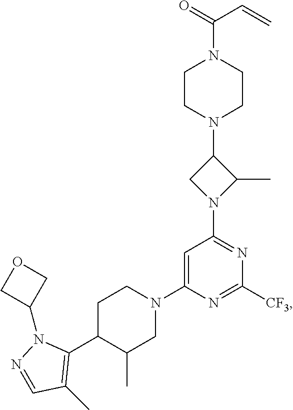 [SMILES: C=CC(=O)N1CCN(C2CN(c3cc(N4CCC(c5c(C)cnn5C5COC5)C(C)C4)nc(C(F)(F)F)n3)C2C)CC1]

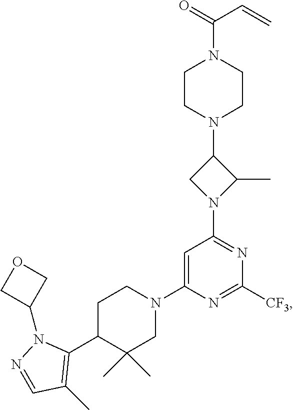 [SMILES: C=CC(=O)N1CCN(C2CN(c3cc(N4CCC(c5c(C)cnn5C5COC5)C(C)(C)C4)nc(C(F)(F)F)n3)C2C)CC1]

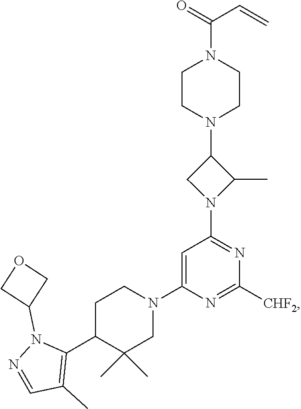 [SMILES: C=CC(=O)N1CCN(C2CN(c3cc(N4CCC(c5c(C)cnn5C5COC5)C(C)(C)C4)nc(C(F)F)n3)C2C)CC1]

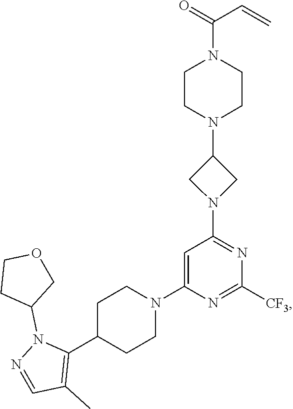 [SMILES: C=CC(=O)N1CCN(C2CN(c3cc(N4CCC(c5c(C)cnn5C5CCOC5)CC4)nc(C(F)(F)F)n3)C2)CC1]

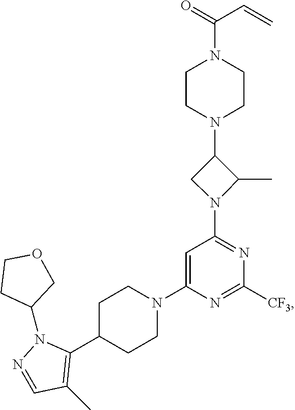 [SMILES: C=CC(=O)N1CCN(C2CN(c3cc(N4CCC(c5c(C)cnn5C5CCOC5)CC4)nc(C(F)(F)F)n3)C2C)CC1]

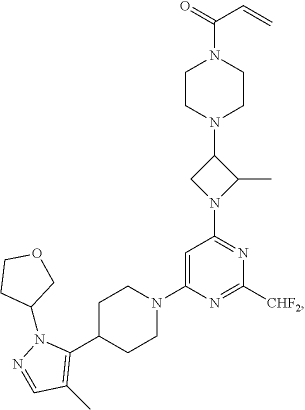 [SMILES: C=CC(=O)N1CCN(C2CN(c3cc(N4CCC(c5c(C)cnn5C5CCOC5)CC4)nc(C(F)F)n3)C2C)CC1]

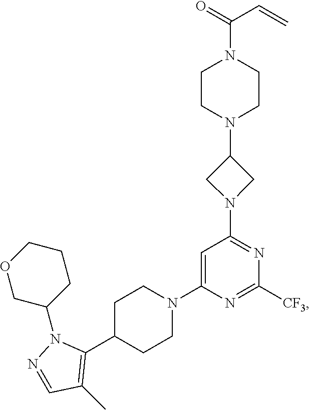 [SMILES: C=CC(=O)N1CCN(C2CN(c3cc(N4CCC(c5c(C)cnn5C5CCCOC5)CC4)nc(C(F)(F)F)n3)C2)CC1]

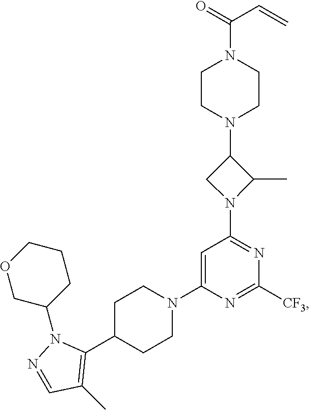 [SMILES: C=CC(=O)N1CCN(C2CN(c3cc(N4CCC(c5c(C)cnn5C5CCCOC5)CC4)nc(C(F)(F)F)n3)C2C)CC1]

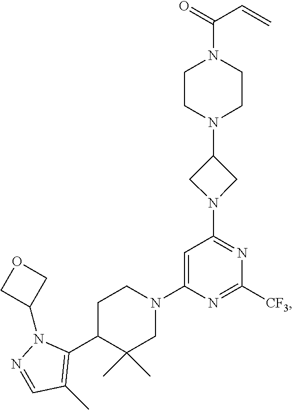 [SMILES: C=CC(=O)N1CCN(C2CN(c3cc(N4CCC(c5c(C)cnn5C5COC5)C(C)(C)C4)nc(C(F)(F)F)n3)C2)CC1]

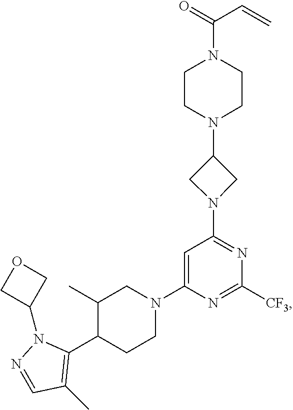 [SMILES: C=CC(=O)N1CCN(C2CN(c3cc(N4CCC(c5c(C)cnn5C5COC5)C(C)C4)nc(C(F)(F)F)n3)C2)CC1]

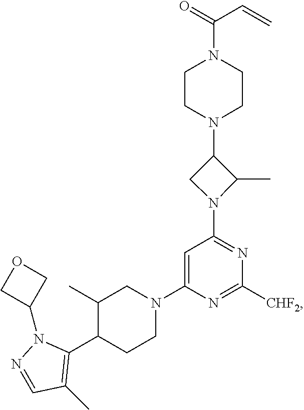 [SMILES: C=CC(=O)N1CCN(C2CN(c3cc(N4CCC(c5c(C)cnn5C5COC5)C(C)C4)nc(C(F)F)n3)C2C)CC1]

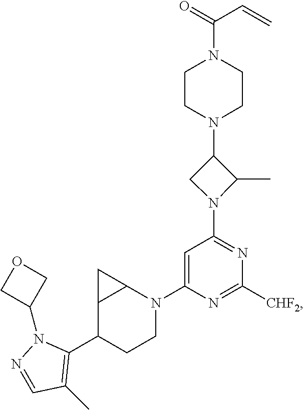 [SMILES: C=CC(=O)N1CCN(C2CN(c3cc(N4CCC(c5c(C)cnn5C5COC5)C5CC54)nc(C(F)F)n3)C2C)CC1]

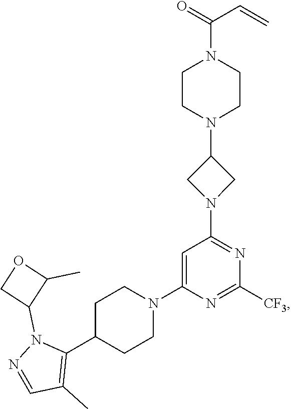 [SMILES: C=CC(=O)N1CCN(C2CN(c3cc(N4CCC(c5c(C)cnn5C5COC5C)CC4)nc(C(F)(F)F)n3)C2)CC1]

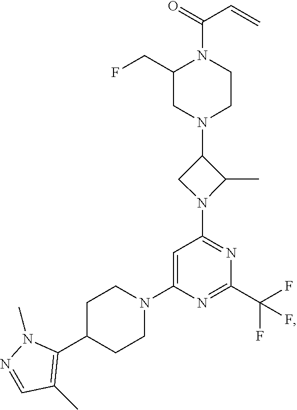 [SMILES: C=CC(=O)N1CCN(C2CN(c3cc(N4CCC(c5c(C)cnn5C)CC4)nc(C(F)(F)F)n3)C2C)CC1CF]

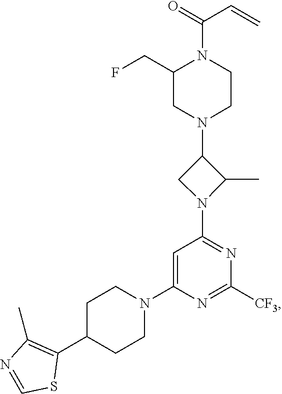 [SMILES: C=CC(=O)N1CCN(C2CN(c3cc(N4CCC(c5scnc5C)CC4)nc(C(F)(F)F)n3)C2C)CC1CF]

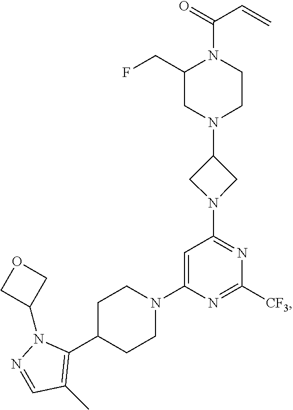 [SMILES: C=CC(=O)N1CCN(C2CN(c3cc(N4CCC(c5c(C)cnn5C5COC5)CC4)nc(C(F)(F)F)n3)C2)CC1CF]

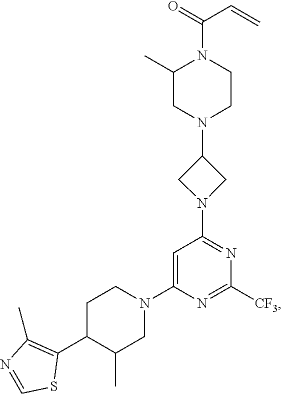 [SMILES: C=CC(=O)N1CCN(C2CN(c3cc(N4CCC(c5scnc5C)C(C)C4)nc(C(F)(F)F)n3)C2)CC1C]

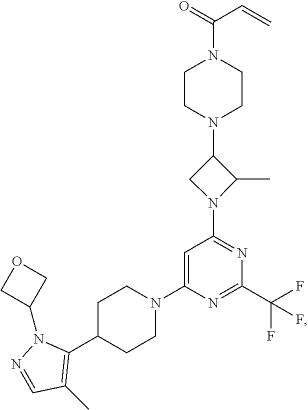 [SMILES: C=CC(=O)N1CCN(C2CN(c3cc(N4CCC(c5c(C)cnn5C5COC5)CC4)nc(C(F)(F)F)n3)C2C)CC1]

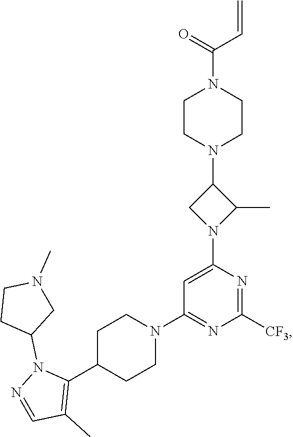 [SMILES: C=CC(=O)N1CCN(C2CN(c3cc(N4CCC(c5c(C)cnn5C5CCN(C)C5)CC4)nc(C(F)(F)F)n3)C2C)CC1]

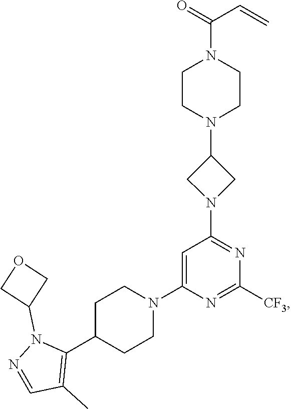 [SMILES: C=CC(=O)N1CCN(C2CN(c3cc(N4CCC(c5c(C)cnn5C5COC5)CC4)nc(C(F)(F)F)n3)C2)CC1]

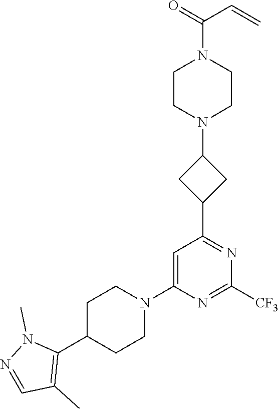 [SMILES: C=CC(=O)N1CCN(C2CC(c3cc(N4CCC(c5c(C)cnn5C)CC4)nc(C(F)(F)F)n3)C2)CC1]

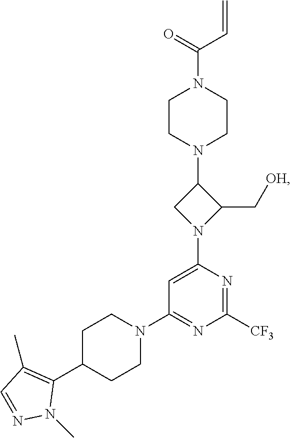 [SMILES: C=CC(=O)N1CCN(C2CN(c3cc(N4CCC(c5c(C)cnn5C)CC4)nc(C(F)(F)F)n3)C2CO)CC1]

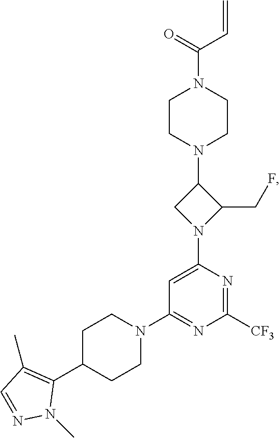 [SMILES: C=CC(=O)N1CCN(C2CN(c3cc(N4CCC(c5c(C)cnn5C)CC4)nc(C(F)(F)F)n3)C2CF)CC1]

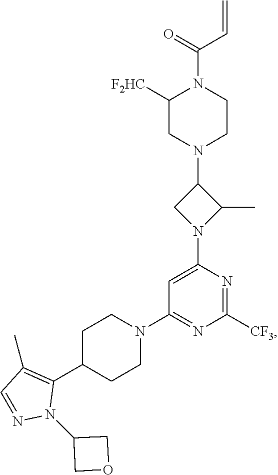 [SMILES: C=CC(=O)N1CCN(C2CN(c3cc(N4CCC(c5c(C)cnn5C5COC5)CC4)nc(C(F)(F)F)n3)C2C)CC1C(F)F]

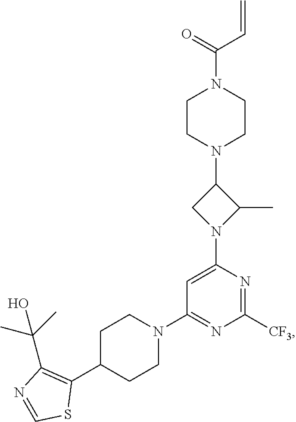 [SMILES: C=CC(=O)N1CCN(C2CN(c3cc(N4CCC(c5scnc5C(C)(C)O)CC4)nc(C(F)(F)F)n3)C2C)CC1]

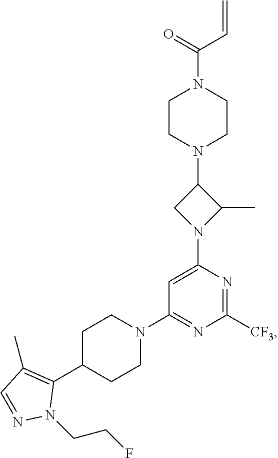 [SMILES: C=CC(=O)N1CCN(C2CN(c3cc(N4CCC(c5c(C)cnn5CCF)CC4)nc(C(F)(F)F)n3)C2C)CC1]

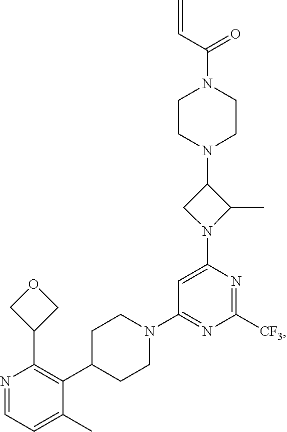 [SMILES: C=CC(=O)N1CCN(C2CN(c3cc(N4CCC(c5c(C)ccnc5C5COC5)CC4)nc(C(F)(F)F)n3)C2C)CC1]

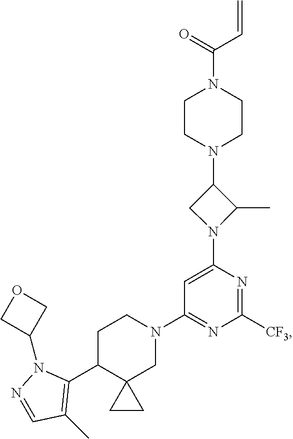 [SMILES: C=CC(=O)N1CCN(C2CN(c3cc(N4CCC(c5c(C)cnn5C5COC5)C5(CC5)C4)nc(C(F)(F)F)n3)C2C)CC1]

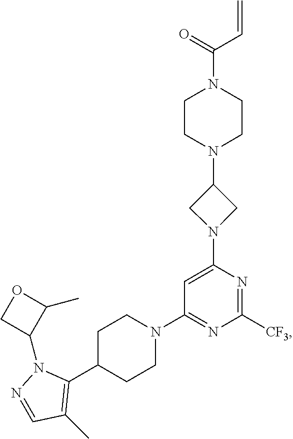 [SMILES: C=CC(=O)N1CCN(C2CN(c3cc(N4CCC(c5c(C)cnn5C5COC5C)CC4)nc(C(F)(F)F)n3)C2)CC1]

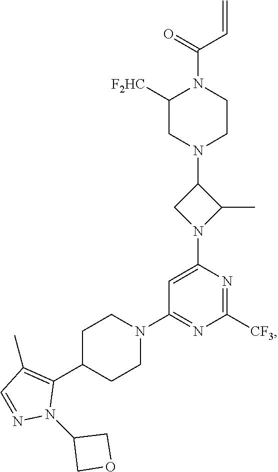 [SMILES: C=CC(=O)N1CCN(C2CN(c3cc(N4CCC(c5c(C)cnn5C5COC5)CC4)nc(C(F)(F)F)n3)C2C)CC1C(F)F]

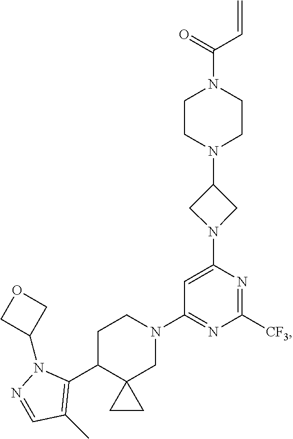 [SMILES: C=CC(=O)N1CCN(C2CN(c3cc(N4CCC(c5c(C)cnn5C5COC5)C5(CC5)C4)nc(C(F)(F)F)n3)C2)CC1]

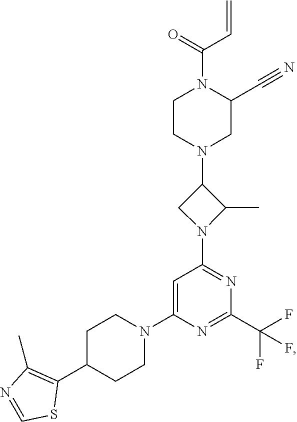 [SMILES: C=CC(=O)N1CCN(C2CN(c3cc(N4CCC(c5scnc5C)CC4)nc(C(F)(F)F)n3)C2C)CC1C#N]

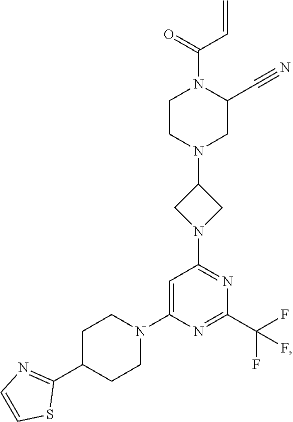 [SMILES: C=CC(=O)N1CCN(C2CN(c3cc(N4CCC(c5nccs5)CC4)nc(C(F)(F)F)n3)C2)CC1C#N]

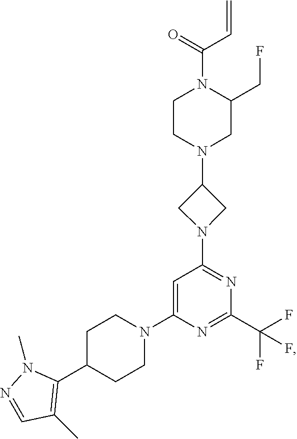 [SMILES: C=CC(=O)N1CCN(C2CN(c3cc(N4CCC(c5c(C)cnn5C)CC4)nc(C(F)(F)F)n3)C2)CC1CF]

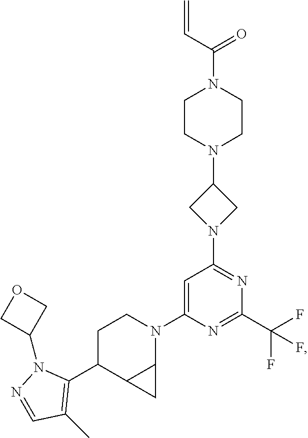 [SMILES: C=CC(=O)N1CCN(C2CN(c3cc(N4CCC(c5c(C)cnn5C5COC5)C5CC54)nc(C(F)(F)F)n3)C2)CC1]

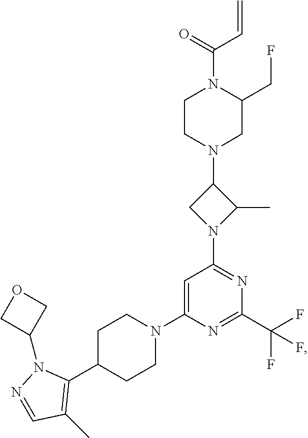 [SMILES: C=CC(=O)N1CCN(C2CN(c3cc(N4CCC(c5c(C)cnn5C5COC5)CC4)nc(C(F)(F)F)n3)C2C)CC1CF]

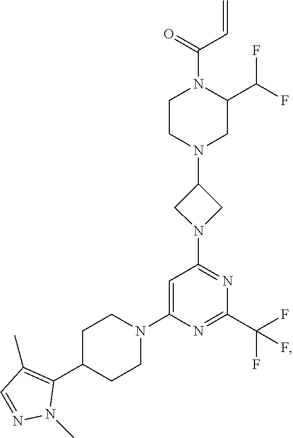 [SMILES: C=CC(=O)N1CCN(C2CN(c3cc(N4CCC(c5c(C)cnn5C)CC4)nc(C(F)(F)F)n3)C2)CC1C(F)F]

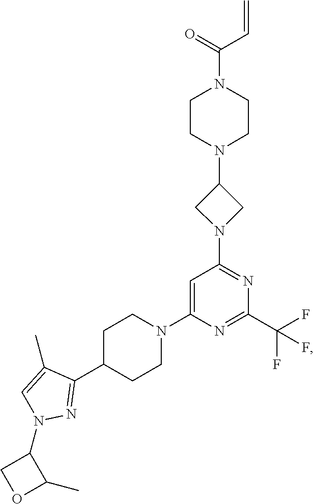 [SMILES: C=CC(=O)N1CCN(C2CN(c3cc(N4CCC(c5nn(C6COC6C)cc5C)CC4)nc(C(F)(F)F)n3)C2)CC1]

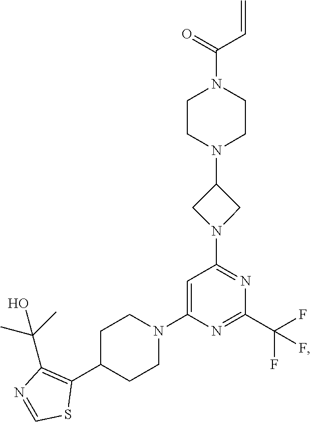 [SMILES: C=CC(=O)N1CCN(C2CN(c3cc(N4CCC(c5scnc5C(C)(C)O)CC4)nc(C(F)(F)F)n3)C2)CC1]

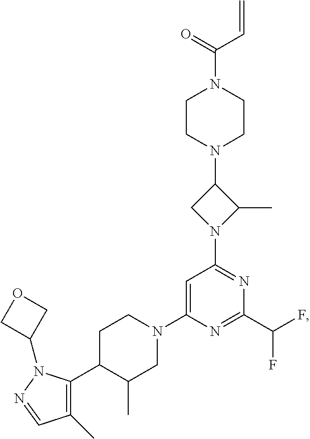 [SMILES: C=CC(=O)N1CCN(C2CN(c3cc(N4CCC(c5c(C)cnn5C5COC5)C(C)C4)nc(C(F)F)n3)C2C)CC1]

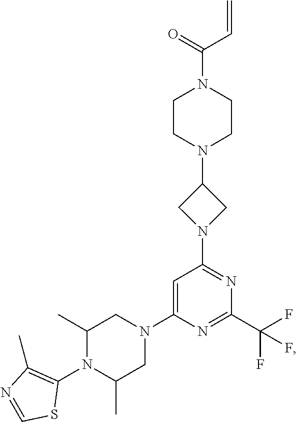 [SMILES: C=CC(=O)N1CCN(C2CN(c3cc(N4CC(C)N(c5scnc5C)C(C)C4)nc(C(F)(F)F)n3)C2)CC1]

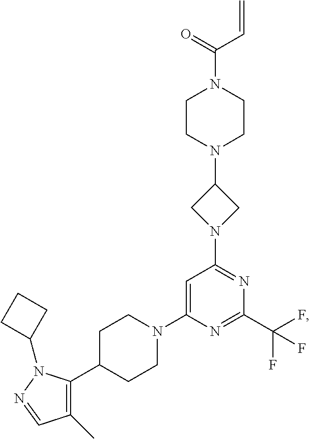 [SMILES: C=CC(=O)N1CCN(C2CN(c3cc(N4CCC(c5c(C)cnn5C5CCC5)CC4)nc(C(F)(F)F)n3)C2)CC1]

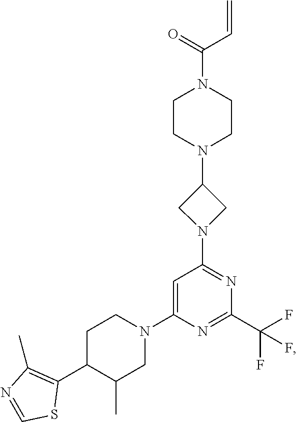 [SMILES: C=CC(=O)N1CCN(C2CN(c3cc(N4CCC(c5scnc5C)C(C)C4)nc(C(F)(F)F)n3)C2)CC1]

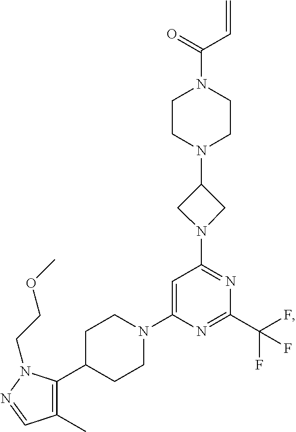 [SMILES: C=CC(=O)N1CCN(C2CN(c3cc(N4CCC(c5c(C)cnn5CCOC)CC4)nc(C(F)(F)F)n3)C2)CC1]

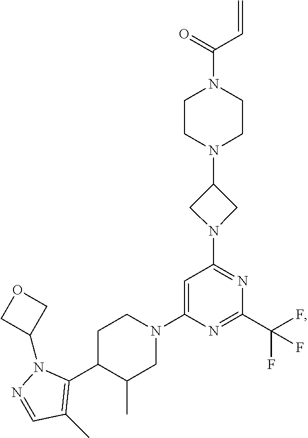 [SMILES: C=CC(=O)N1CCN(C2CN(c3cc(N4CCC(c5c(C)cnn5C5COC5)C(C)C4)nc(C(F)(F)F)n3)C2)CC1]

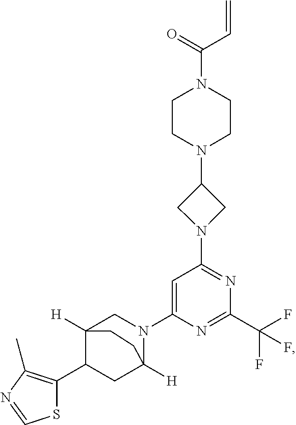 [SMILES: C=CC(=O)N1CCN(C2CN(c3cc(N4CC5CCC4CC5c4scnc4C)nc(C(F)(F)F)n3)C2)CC1]

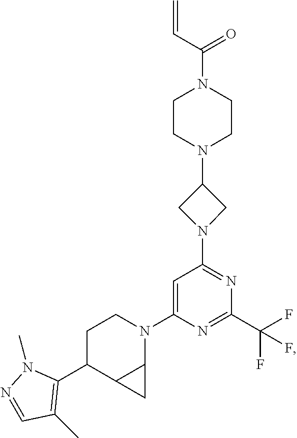 [SMILES: C=CC(=O)N1CCN(C2CN(c3cc(N4CCC(c5c(C)cnn5C)C5CC54)nc(C(F)(F)F)n3)C2)CC1]

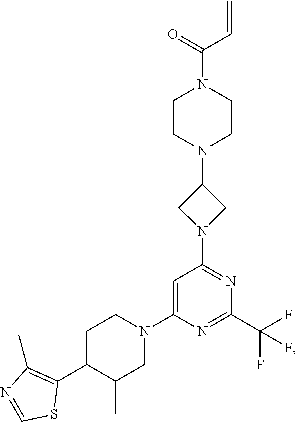 [SMILES: C=CC(=O)N1CCN(C2CN(c3cc(N4CCC(c5scnc5C)C(C)C4)nc(C(F)(F)F)n3)C2)CC1]

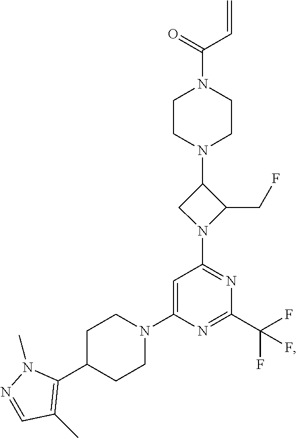 [SMILES: C=CC(=O)N1CCN(C2CN(c3cc(N4CCC(c5c(C)cnn5C)CC4)nc(C(F)(F)F)n3)C2CF)CC1]

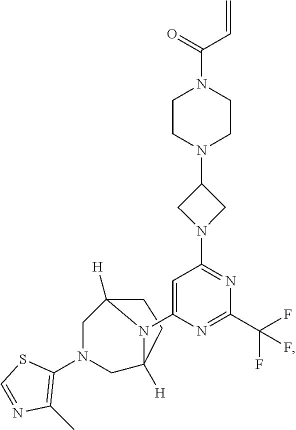 [SMILES: C=CC(=O)N1CCN(C2CN(c3cc(N4C5CCC4CN(c4scnc4C)C5)nc(C(F)(F)F)n3)C2)CC1]

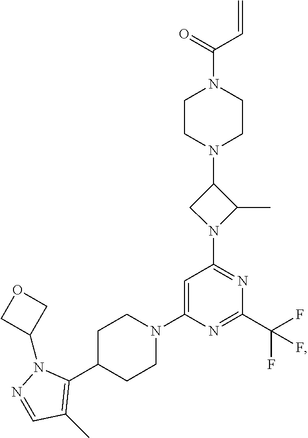 [SMILES: C=CC(=O)N1CCN(C2CN(c3cc(N4CCC(c5c(C)cnn5C5COC5)CC4)nc(C(F)(F)F)n3)C2C)CC1]

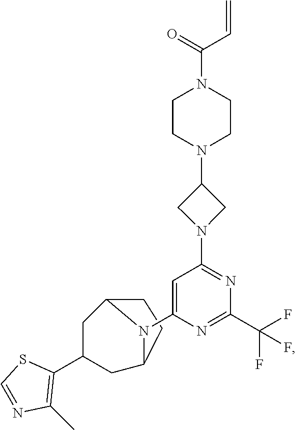 [SMILES: C=CC(=O)N1CCN(C2CN(c3cc(N4C5CCC4CC(c4scnc4C)C5)nc(C(F)(F)F)n3)C2)CC1]

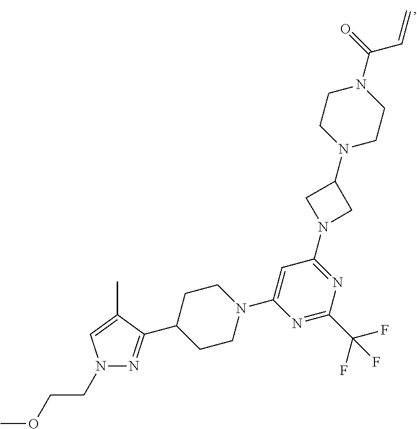 [SMILES: C=CC(=O)N1CCN(C2CN(c3cc(N4CCC(c5nn(CCOC)cc5C)CC4)nc(C(F)(F)F)n3)C2)CC1]

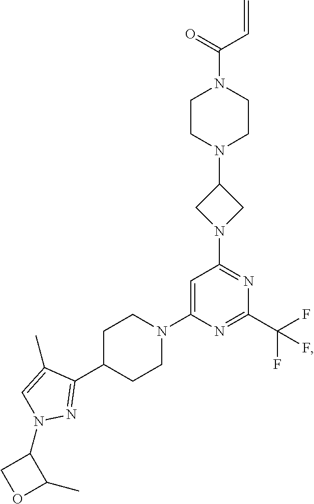 [SMILES: C=CC(=O)N1CCN(C2CN(c3cc(N4CCC(c5nn(C6COC6C)cc5C)CC4)nc(C(F)(F)F)n3)C2)CC1]

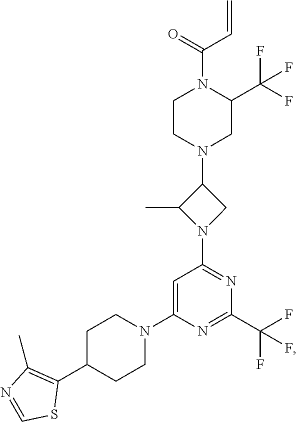 [SMILES: C=CC(=O)N1CCN(C2CN(c3cc(N4CCC(c5scnc5C)CC4)nc(C(F)(F)F)n3)C2C)CC1C(F)(F)F]

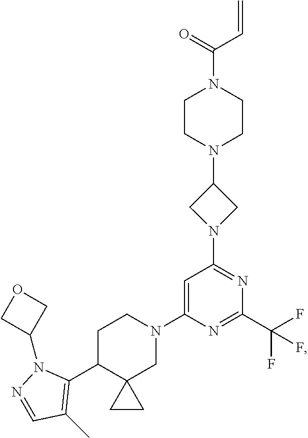 [SMILES: C=CC(=O)N1CCN(C2CN(c3cc(N4CCC(c5c(C)cnn5C5COC5)C5(CC5)C4)nc(C(F)(F)F)n3)C2)CC1]

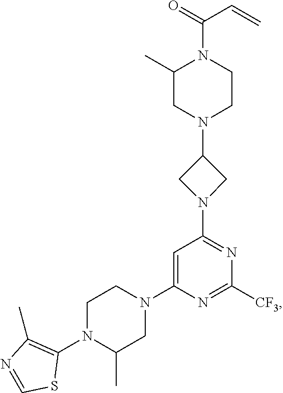 [SMILES: C=CC(=O)N1CCN(C2CN(c3cc(N4CCN(c5scnc5C)C(C)C4)nc(C(F)(F)F)n3)C2)CC1C]

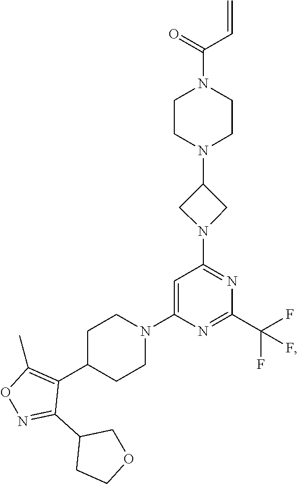 [SMILES: C=CC(=O)N1CCN(C2CN(c3cc(N4CCC(c5c(C6CCOC6)noc5C)CC4)nc(C(F)(F)F)n3)C2)CC1]

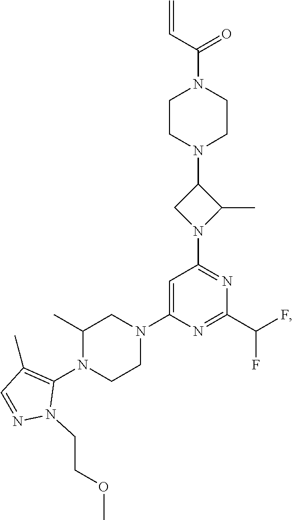 [SMILES: C=CC(=O)N1CCN(C2CN(c3cc(N4CCN(c5c(C)cnn5CCOC)C(C)C4)nc(C(F)F)n3)C2C)CC1]

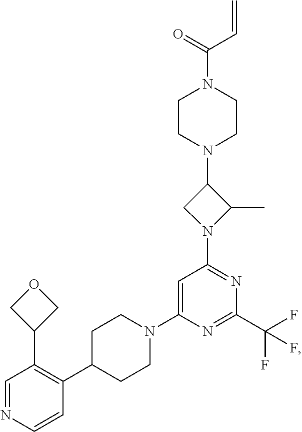 [SMILES: C=CC(=O)N1CCN(C2CN(c3cc(N4CCC(c5ccncc5C5COC5)CC4)nc(C(F)(F)F)n3)C2C)CC1]

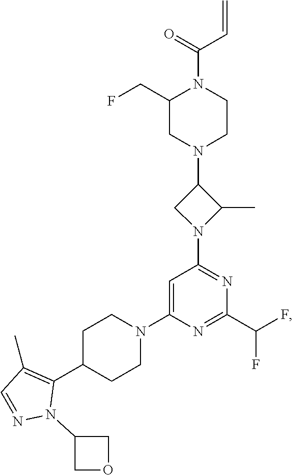 [SMILES: C=CC(=O)N1CCN(C2CN(c3cc(N4CCC(c5c(C)cnn5C5COC5)CC4)nc(C(F)F)n3)C2C)CC1CF]

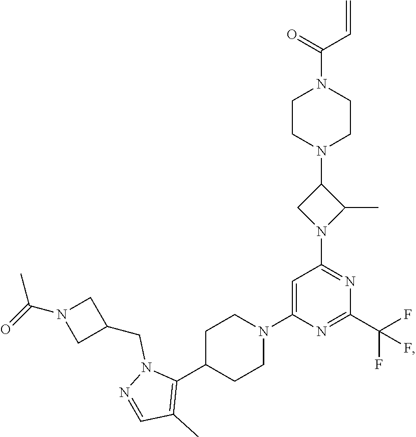 [SMILES: C=CC(=O)N1CCN(C2CN(c3cc(N4CCC(c5c(C)cnn5CC5CN(C(C)=O)C5)CC4)nc(C(F)(F)F)n3)C2C)CC1]

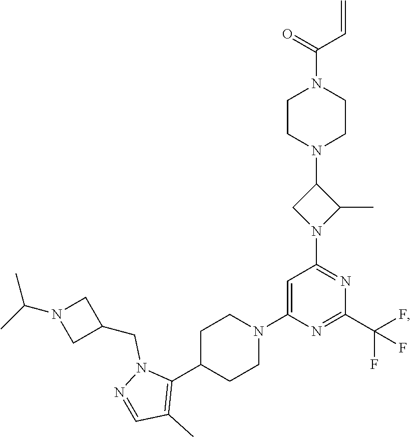 [SMILES: C=CC(=O)N1CCN(C2CN(c3cc(N4CCC(c5c(C)cnn5CC5CN(C(C)C)C5)CC4)nc(C(F)(F)F)n3)C2C)CC1]

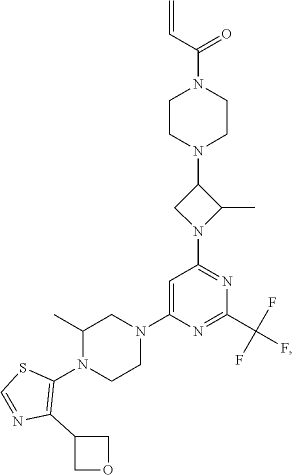 [SMILES: C=CC(=O)N1CCN(C2CN(c3cc(N4CCN(c5scnc5C5COC5)C(C)C4)nc(C(F)(F)F)n3)C2C)CC1]

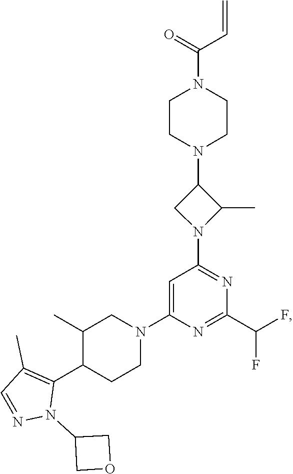 [SMILES: C=CC(=O)N1CCN(C2CN(c3cc(N4CCC(c5c(C)cnn5C5COC5)C(C)C4)nc(C(F)F)n3)C2C)CC1]

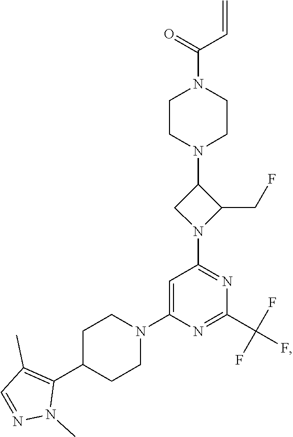 [SMILES: C=CC(=O)N1CCN(C2CN(c3cc(N4CCC(c5c(C)cnn5C)CC4)nc(C(F)(F)F)n3)C2CF)CC1]

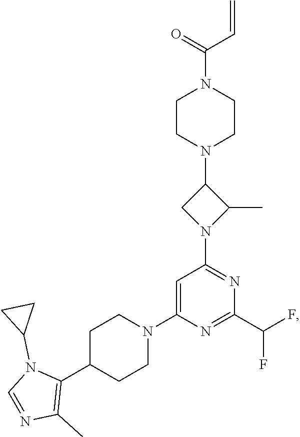 [SMILES: C=CC(=O)N1CCN(C2CN(c3cc(N4CCC(c5c(C)ncn5C5CC5)CC4)nc(C(F)F)n3)C2C)CC1]

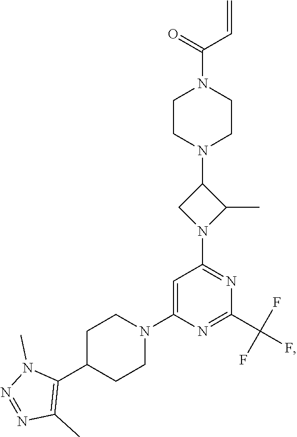 [SMILES: C=CC(=O)N1CCN(C2CN(c3cc(N4CCC(c5c(C)nnn5C)CC4)nc(C(F)(F)F)n3)C2C)CC1]

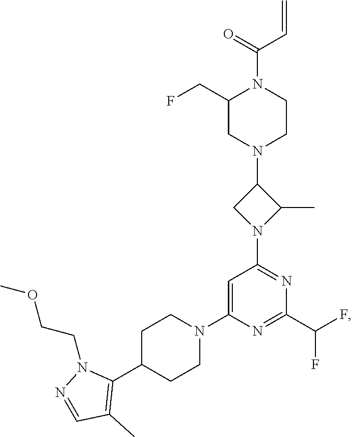 [SMILES: C=CC(=O)N1CCN(C2CN(c3cc(N4CCC(c5c(C)cnn5CCOC)CC4)nc(C(F)F)n3)C2C)CC1CF]

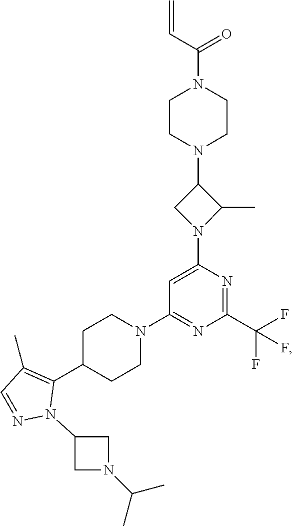 [SMILES: C=CC(=O)N1CCN(C2CN(c3cc(N4CCC(c5c(C)cnn5C5CN(C(C)C)C5)CC4)nc(C(F)(F)F)n3)C2C)CC1]

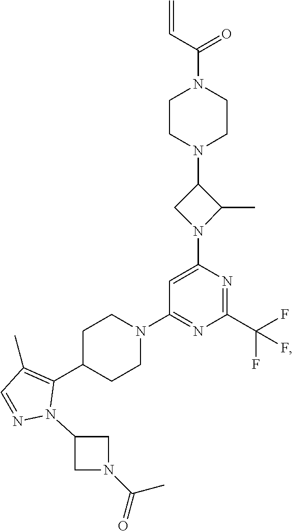 [SMILES: C=CC(=O)N1CCN(C2CN(c3cc(N4CCC(c5c(C)cnn5C5CN(C(C)=O)C5)CC4)nc(C(F)(F)F)n3)C2C)CC1]

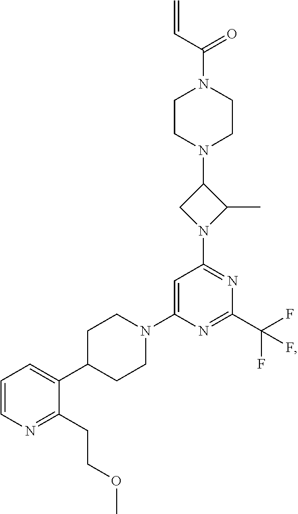 [SMILES: C=CC(=O)N1CCN(C2CN(c3cc(N4CCC(c5cccnc5CCOC)CC4)nc(C(F)(F)F)n3)C2C)CC1]

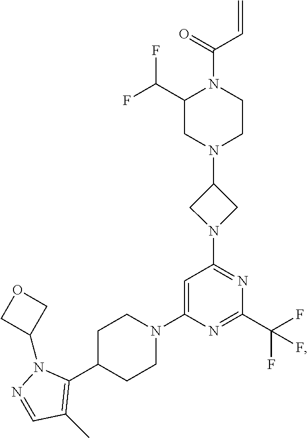 [SMILES: C=CC(=O)N1CCN(C2CN(c3cc(N4CCC(c5c(C)cnn5C5COC5)CC4)nc(C(F)(F)F)n3)C2)CC1C(F)F]

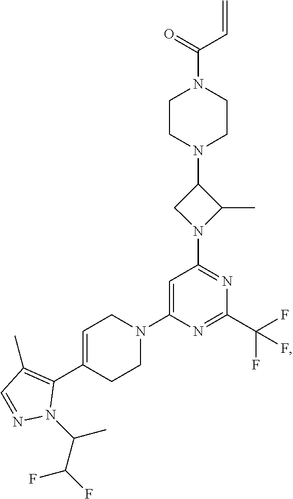 [SMILES: C=CC(=O)N1CCN(C2CN(c3cc(N4CC=C(c5c(C)cnn5C(C)C(F)F)CC4)nc(C(F)(F)F)n3)C2C)CC1]

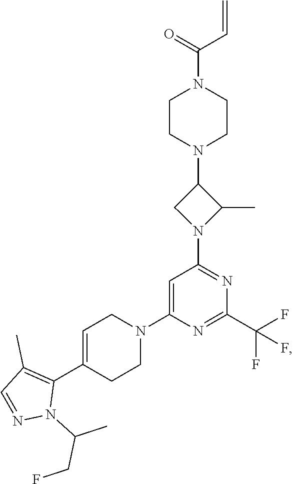 [SMILES: C=CC(=O)N1CCN(C2CN(c3cc(N4CC=C(c5c(C)cnn5C(C)CF)CC4)nc(C(F)(F)F)n3)C2C)CC1]

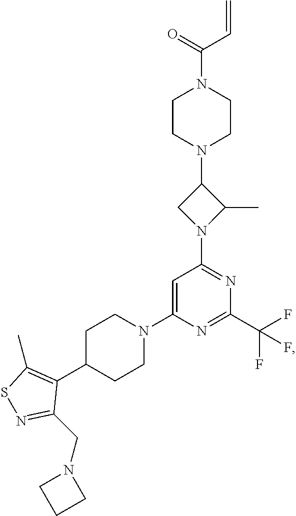 [SMILES: C=CC(=O)N1CCN(C2CN(c3cc(N4CCC(c5c(CN6CCC6)nsc5C)CC4)nc(C(F)(F)F)n3)C2C)CC1]

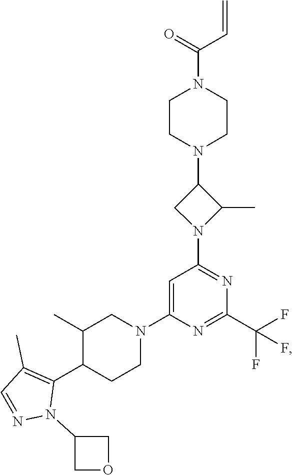 [SMILES: C=CC(=O)N1CCN(C2CN(c3cc(N4CCC(c5c(C)cnn5C5COC5)C(C)C4)nc(C(F)(F)F)n3)C2C)CC1]

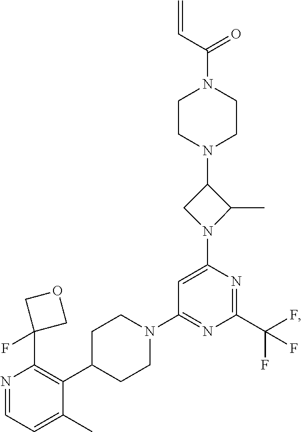 [SMILES: C=CC(=O)N1CCN(C2CN(c3cc(N4CCC(c5c(C)ccnc5C5(F)COC5)CC4)nc(C(F)(F)F)n3)C2C)CC1]

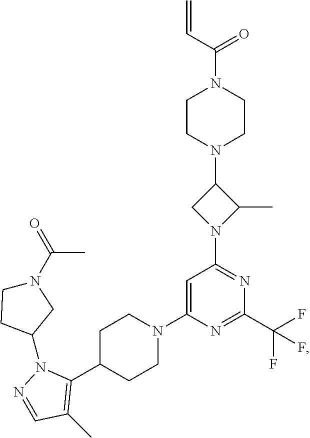 [SMILES: C=CC(=O)N1CCN(C2CN(c3cc(N4CCC(c5c(C)cnn5C5CCN(C(C)=O)C5)CC4)nc(C(F)(F)F)n3)C2C)CC1]

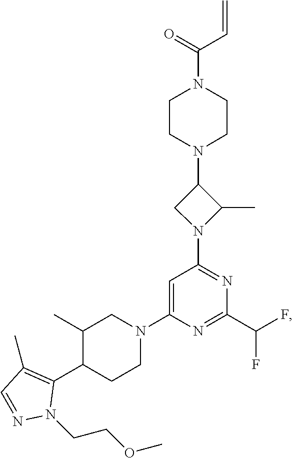 [SMILES: C=CC(=O)N1CCN(C2CN(c3cc(N4CCC(c5c(C)cnn5CCOC)C(C)C4)nc(C(F)F)n3)C2C)CC1]

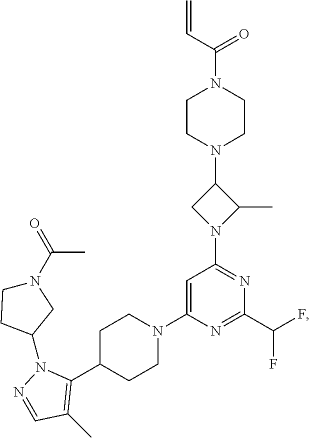 [SMILES: C=CC(=O)N1CCN(C2CN(c3cc(N4CCC(c5c(C)cnn5C5CCN(C(C)=O)C5)CC4)nc(C(F)F)n3)C2C)CC1]

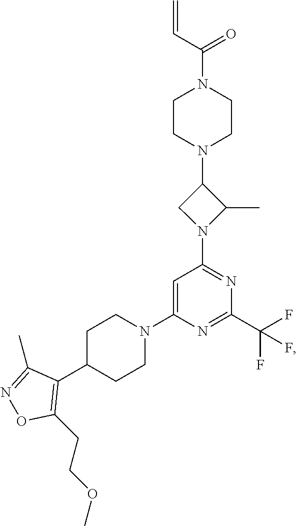 [SMILES: C=CC(=O)N1CCN(C2CN(c3cc(N4CCC(c5c(C)noc5CCOC)CC4)nc(C(F)(F)F)n3)C2C)CC1]

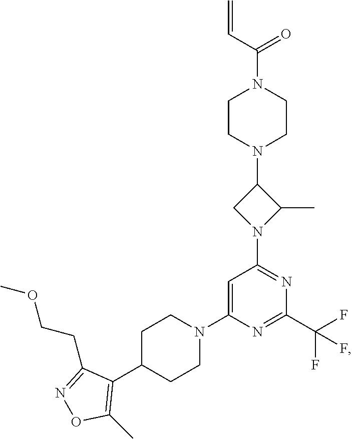 [SMILES: C=CC(=O)N1CCN(C2CN(c3cc(N4CCC(c5c(CCOC)noc5C)CC4)nc(C(F)(F)F)n3)C2C)CC1]

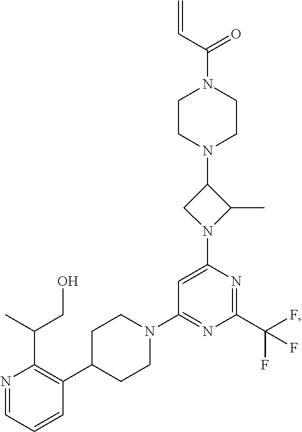 [SMILES: C=CC(=O)N1CCN(C2CN(c3cc(N4CCC(c5cccnc5C(C)CO)CC4)nc(C(F)(F)F)n3)C2C)CC1]

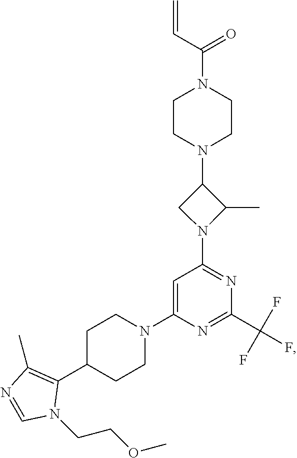 [SMILES: C=CC(=O)N1CCN(C2CN(c3cc(N4CCC(c5c(C)ncn5CCOC)CC4)nc(C(F)(F)F)n3)C2C)CC1]

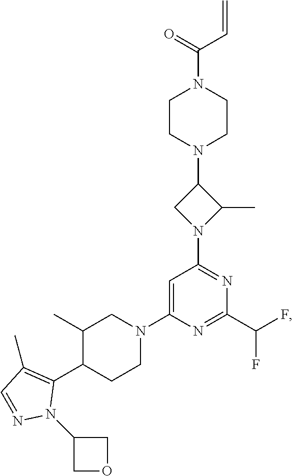 [SMILES: C=CC(=O)N1CCN(C2CN(c3cc(N4CCC(c5c(C)cnn5C5COC5)C(C)C4)nc(C(F)F)n3)C2C)CC1]

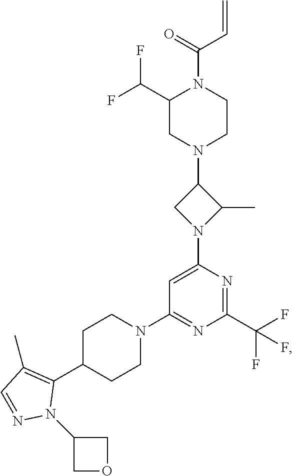 [SMILES: C=CC(=O)N1CCN(C2CN(c3cc(N4CCC(c5c(C)cnn5C5COC5)CC4)nc(C(F)(F)F)n3)C2C)CC1C(F)F]

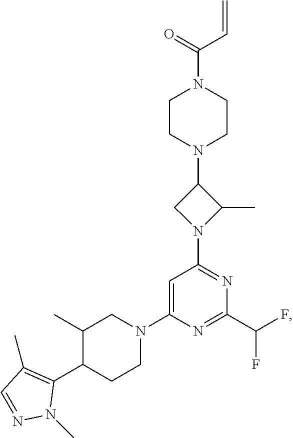 [SMILES: C=CC(=O)N1CCN(C2CN(c3cc(N4CCC(c5c(C)cnn5C)C(C)C4)nc(C(F)F)n3)C2C)CC1]

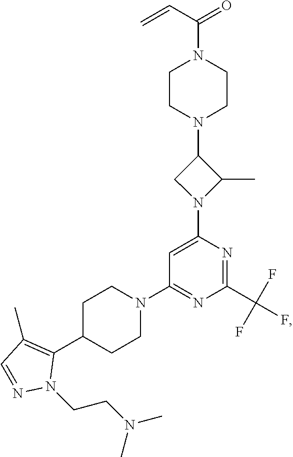 [SMILES: C=CC(=O)N1CCN(C2CN(c3cc(N4CCC(c5c(C)cnn5CCN(C)C)CC4)nc(C(F)(F)F)n3)C2C)CC1]

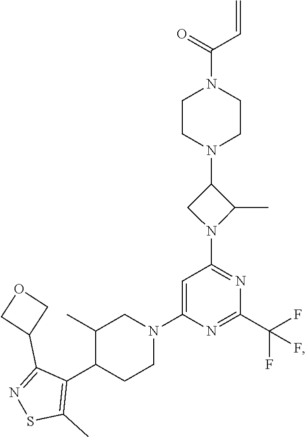 [SMILES: C=CC(=O)N1CCN(C2CN(c3cc(N4CCC(c5c(C6COC6)nsc5C)C(C)C4)nc(C(F)(F)F)n3)C2C)CC1]

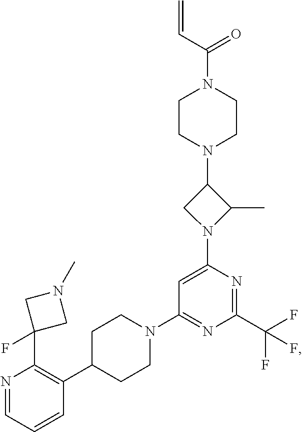 [SMILES: C=CC(=O)N1CCN(C2CN(c3cc(N4CCC(c5cccnc5C5(F)CN(C)C5)CC4)nc(C(F)(F)F)n3)C2C)CC1]

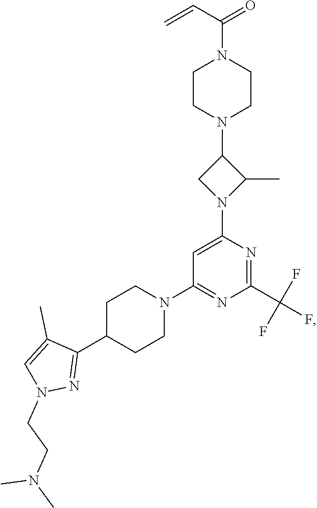 [SMILES: C=CC(=O)N1CCN(C2CN(c3cc(N4CCC(c5nn(CCN(C)C)cc5C)CC4)nc(C(F)(F)F)n3)C2C)CC1]

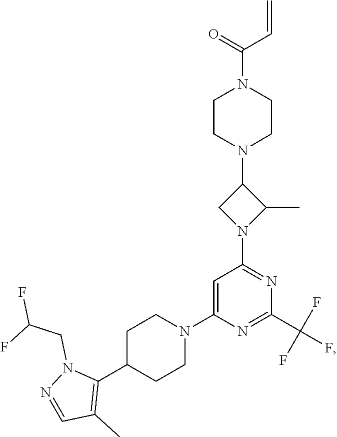 [SMILES: C=CC(=O)N1CCN(C2CN(c3cc(N4CCC(c5c(C)cnn5CC(F)F)CC4)nc(C(F)(F)F)n3)C2C)CC1]

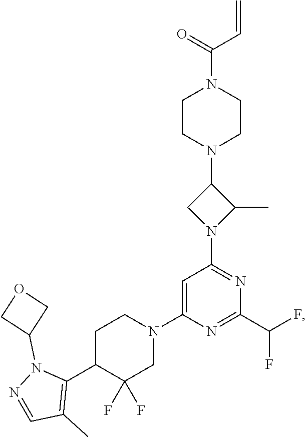 [SMILES: C=CC(=O)N1CCN(C2CN(c3cc(N4CCC(c5c(C)cnn5C5COC5)C(F)(F)C4)nc(C(F)F)n3)C2C)CC1]

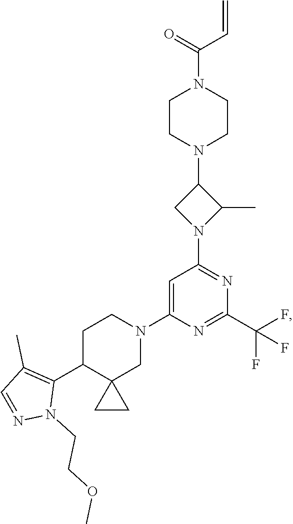 [SMILES: C=CC(=O)N1CCN(C2CN(c3cc(N4CCC(c5c(C)cnn5CCOC)C5(CC5)C4)nc(C(F)(F)F)n3)C2C)CC1]

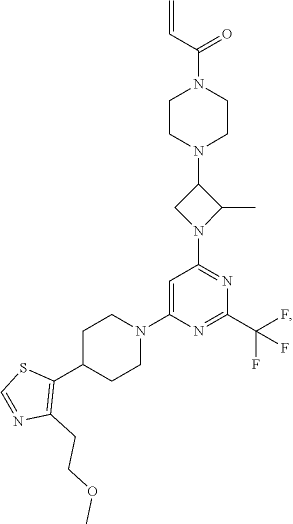 [SMILES: C=CC(=O)N1CCN(C2CN(c3cc(N4CCC(c5scnc5CCOC)CC4)nc(C(F)(F)F)n3)C2C)CC1]

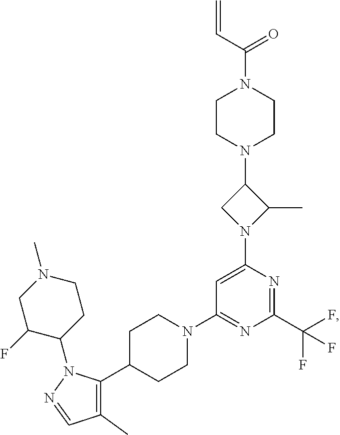 [SMILES: C=CC(=O)N1CCN(C2CN(c3cc(N4CCC(c5c(C)cnn5C5CCN(C)CC5F)CC4)nc(C(F)(F)F)n3)C2C)CC1]

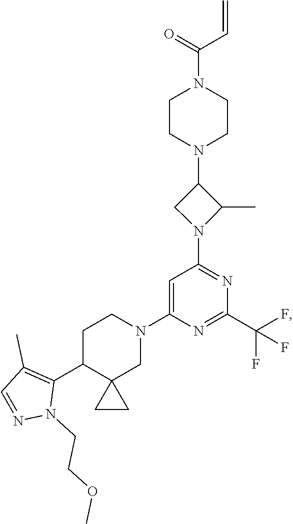 [SMILES: C=CC(=O)N1CCN(C2CN(c3cc(N4CCC(c5c(C)cnn5CCOC)C5(CC5)C4)nc(C(F)(F)F)n3)C2C)CC1]

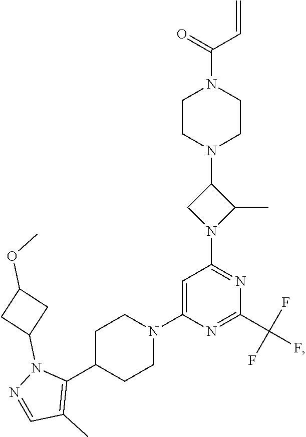 [SMILES: C=CC(=O)N1CCN(C2CN(c3cc(N4CCC(c5c(C)cnn5C5CC(OC)C5)CC4)nc(C(F)(F)F)n3)C2C)CC1]

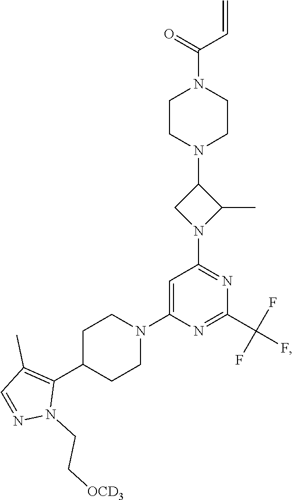

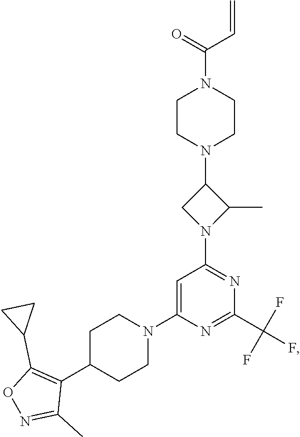 [SMILES: C=CC(=O)N1CCN(C2CN(c3cc(N4CCC(c5c(C)noc5C5CC5)CC4)nc(C(F)(F)F)n3)C2C)CC1]

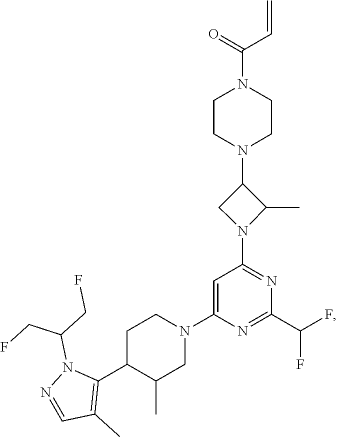 [SMILES: C=CC(=O)N1CCN(C2CN(c3cc(N4CCC(c5c(C)cnn5C(CF)CF)C(C)C4)nc(C(F)F)n3)C2C)CC1]

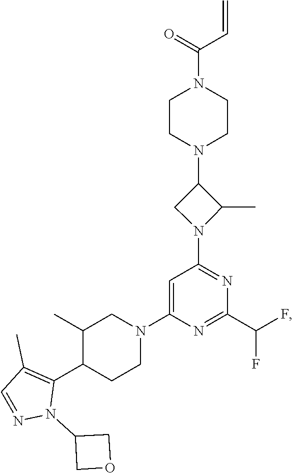 [SMILES: C=CC(=O)N1CCN(C2CN(c3cc(N4CCC(c5c(C)cnn5C5COC5)C(C)C4)nc(C(F)F)n3)C2C)CC1]

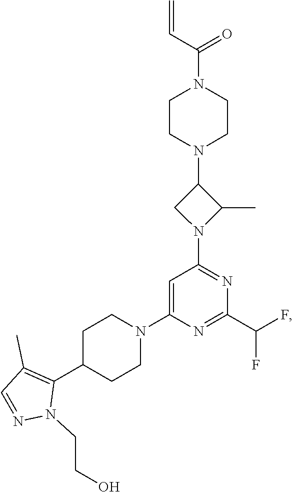 [SMILES: C=CC(=O)N1CCN(C2CN(c3cc(N4CCC(c5c(C)cnn5CCO)CC4)nc(C(F)F)n3)C2C)CC1]

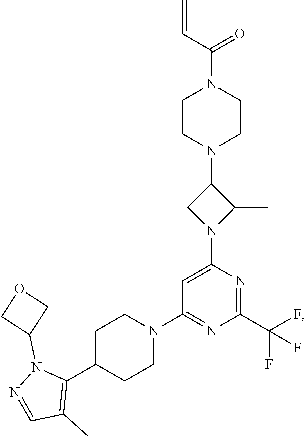 [SMILES: C=CC(=O)N1CCN(C2CN(c3cc(N4CCC(c5c(C)cnn5C5COC5)CC4)nc(C(F)(F)F)n3)C2C)CC1]

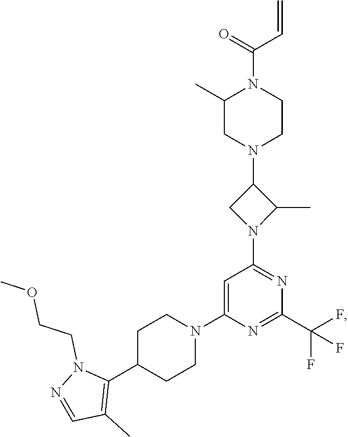 [SMILES: C=CC(=O)N1CCN(C2CN(c3cc(N4CCC(c5c(C)cnn5CCOC)CC4)nc(C(F)(F)F)n3)C2C)CC1C]

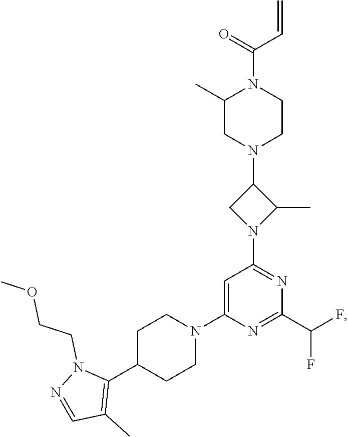 [SMILES: C=CC(=O)N1CCN(C2CN(c3cc(N4CCC(c5c(C)cnn5CCOC)CC4)nc(C(F)F)n3)C2C)CC1C]

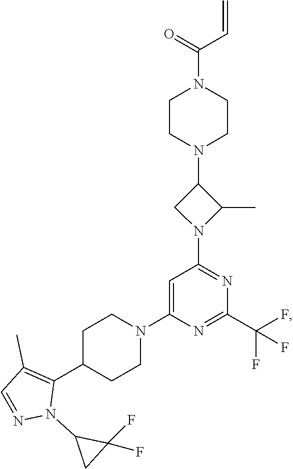 [SMILES: C=CC(=O)N1CCN(C2CN(c3cc(N4CCC(c5c(C)cnn5C5CC5(F)F)CC4)nc(C(F)(F)F)n3)C2C)CC1]

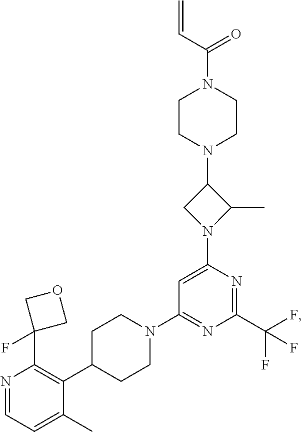 [SMILES: C=CC(=O)N1CCN(C2CN(c3cc(N4CCC(c5c(C)ccnc5C5(F)COC5)CC4)nc(C(F)(F)F)n3)C2C)CC1]

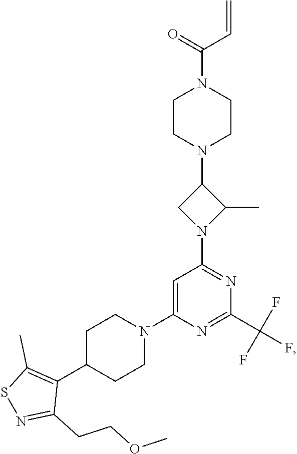 [SMILES: C=CC(=O)N1CCN(C2CN(c3cc(N4CCC(c5c(CCOC)nsc5C)CC4)nc(C(F)(F)F)n3)C2C)CC1]

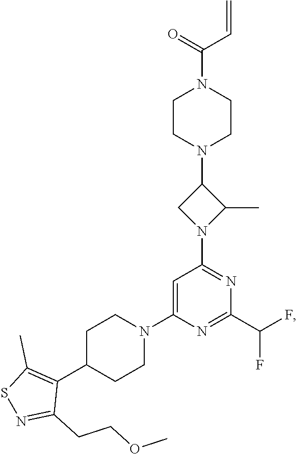 [SMILES: C=CC(=O)N1CCN(C2CN(c3cc(N4CCC(c5c(CCOC)nsc5C)CC4)nc(C(F)F)n3)C2C)CC1]

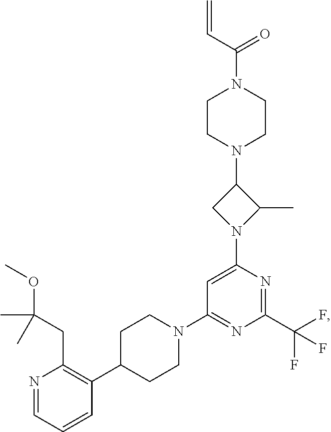 [SMILES: C=CC(=O)N1CCN(C2CN(c3cc(N4CCC(c5cccnc5CC(C)(C)OC)CC4)nc(C(F)(F)F)n3)C2C)CC1]

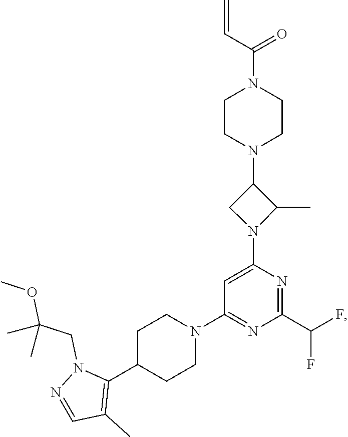 [SMILES: C=CC(=O)N1CCN(C2CN(c3cc(N4CCC(c5c(C)cnn5CC(C)(C)OC)CC4)nc(C(F)F)n3)C2C)CC1]

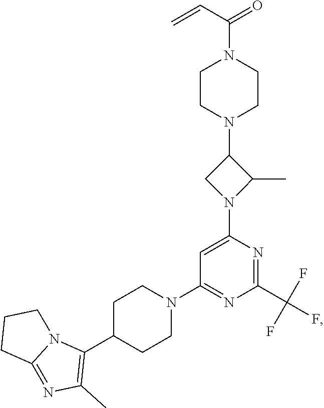 [SMILES: C=CC(=O)N1CCN(C2CN(c3cc(N4CCC(c5c(C)nc6n5CCC6)CC4)nc(C(F)(F)F)n3)C2C)CC1]

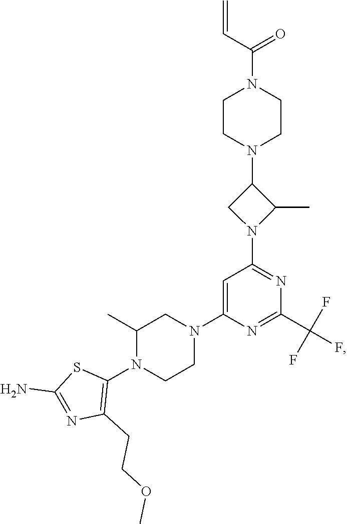 [SMILES: C=CC(=O)N1CCN(C2CN(c3cc(N4CCN(c5sc(N)nc5CCOC)C(C)C4)nc(C(F)(F)F)n3)C2C)CC1]

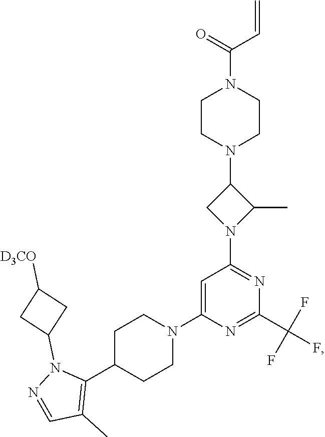

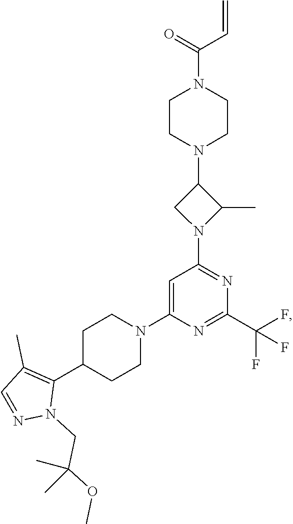 [SMILES: C=CC(=O)N1CCN(C2CN(c3cc(N4CCC(c5c(C)cnn5CC(C)(C)OC)CC4)nc(C(F)(F)F)n3)C2C)CC1]

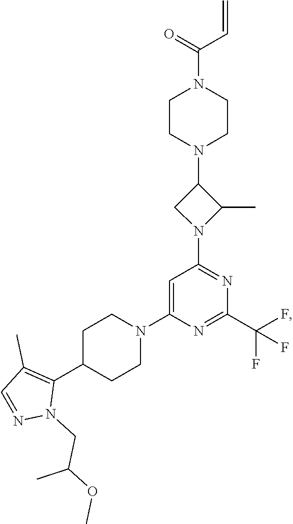 [SMILES: C=CC(=O)N1CCN(C2CN(c3cc(N4CCC(c5c(C)cnn5CC(C)OC)CC4)nc(C(F)(F)F)n3)C2C)CC1]

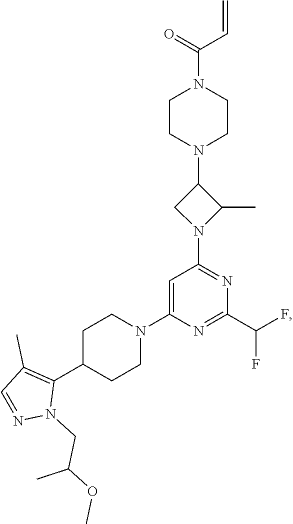 [SMILES: C=CC(=O)N1CCN(C2CN(c3cc(N4CCC(c5c(C)cnn5CC(C)OC)CC4)nc(C(F)F)n3)C2C)CC1]

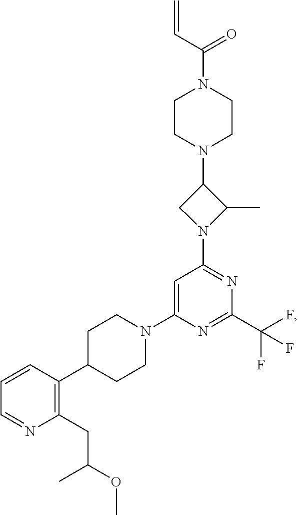 [SMILES: C=CC(=O)N1CCN(C2CN(c3cc(N4CCC(c5cccnc5CC(C)OC)CC4)nc(C(F)(F)F)n3)C2C)CC1]

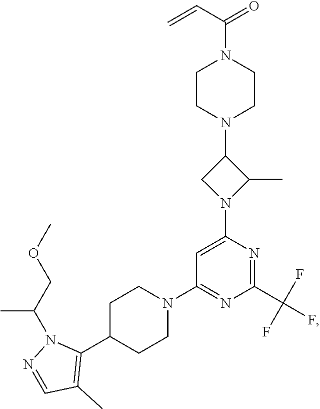 [SMILES: C=CC(=O)N1CCN(C2CN(c3cc(N4CCC(c5c(C)cnn5C(C)COC)CC4)nc(C(F)(F)F)n3)C2C)CC1]

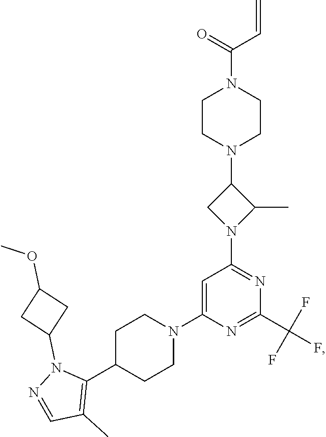 [SMILES: C=CC(=O)N1CCN(C2CN(c3cc(N4CCC(c5c(C)cnn5C5CC(OC)C5)CC4)nc(C(F)(F)F)n3)C2C)CC1]

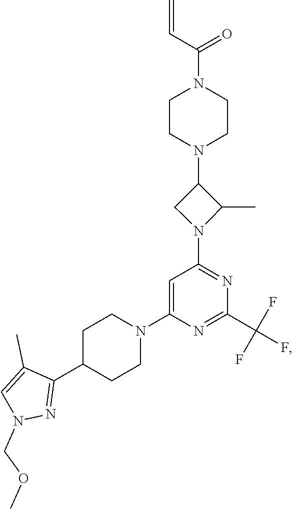 [SMILES: C=CC(=O)N1CCN(C2CN(c3cc(N4CCC(c5nn(COC)cc5C)CC4)nc(C(F)(F)F)n3)C2C)CC1]

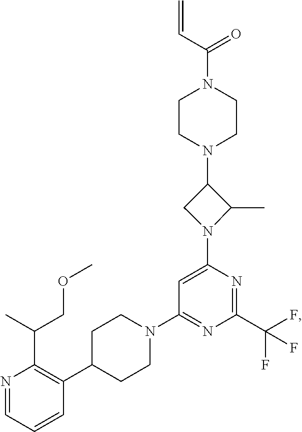 [SMILES: C=CC(=O)N1CCN(C2CN(c3cc(N4CCC(c5cccnc5C(C)COC)CC4)nc(C(F)(F)F)n3)C2C)CC1]

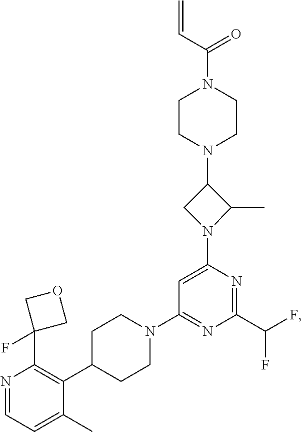 [SMILES: C=CC(=O)N1CCN(C2CN(c3cc(N4CCC(c5c(C)ccnc5C5(F)COC5)CC4)nc(C(F)F)n3)C2C)CC1]

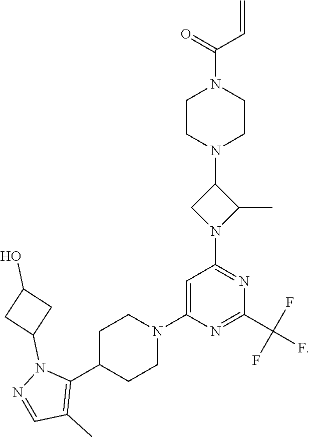 [SMILES: C=CC(=O)N1CCN(C2CN(c3cc(N4CCC(c5c(C)cnn5C5CC(O)C5)CC4)nc(C(F)(F)F)n3)C2C)CC1]

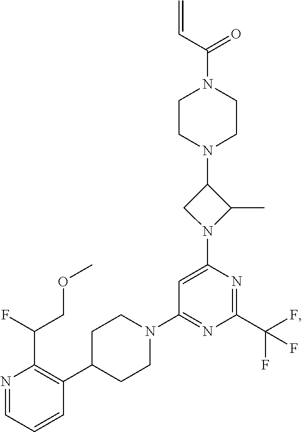 [SMILES: C=CC(=O)N1CCN(C2CN(c3cc(N4CCC(c5cccnc5C(F)COC)CC4)nc(C(F)(F)F)n3)C2C)CC1]

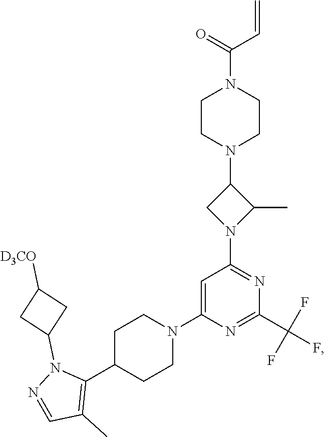

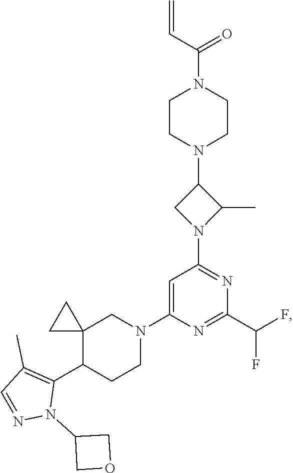 [SMILES: C=CC(=O)N1CCN(C2CN(c3cc(N4CCC(c5c(C)cnn5C5COC5)C5(CC5)C4)nc(C(F)F)n3)C2C)CC1]

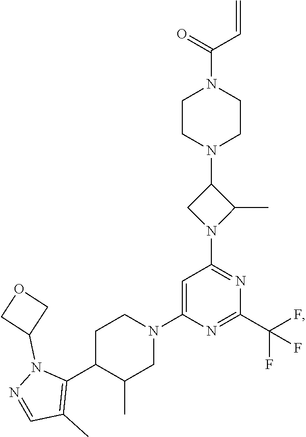 [SMILES: C=CC(=O)N1CCN(C2CN(c3cc(N4CCC(c5c(C)cnn5C5COC5)C(C)C4)nc(C(F)(F)F)n3)C2C)CC1]

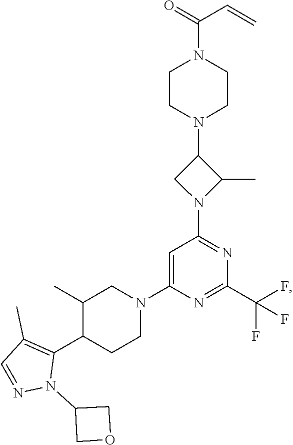 [SMILES: C=CC(=O)N1CCN(C2CN(c3cc(N4CCC(c5c(C)cnn5C5COC5)C(C)C4)nc(C(F)(F)F)n3)C2C)CC1]

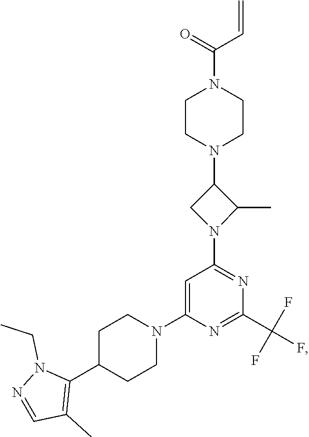 [SMILES: C=CC(=O)N1CCN(C2CN(c3cc(N4CCC(c5c(C)cnn5CC)CC4)nc(C(F)(F)F)n3)C2C)CC1]

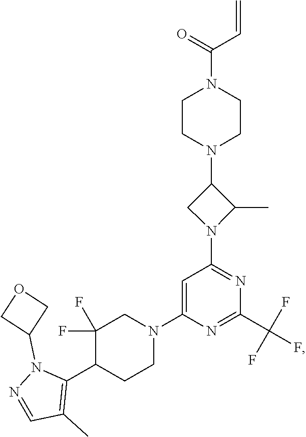 [SMILES: C=CC(=O)N1CCN(C2CN(c3cc(N4CCC(c5c(C)cnn5C5COC5)C(F)(F)C4)nc(C(F)(F)F)n3)C2C)CC1]

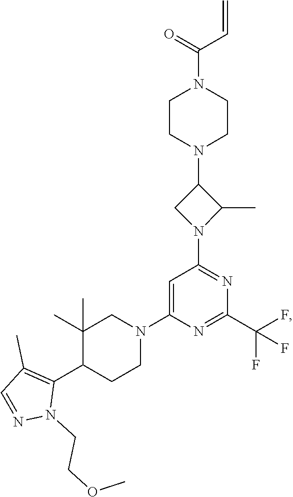 [SMILES: C=CC(=O)N1CCN(C2CN(c3cc(N4CCC(c5c(C)cnn5CCOC)C(C)(C)C4)nc(C(F)(F)F)n3)C2C)CC1]

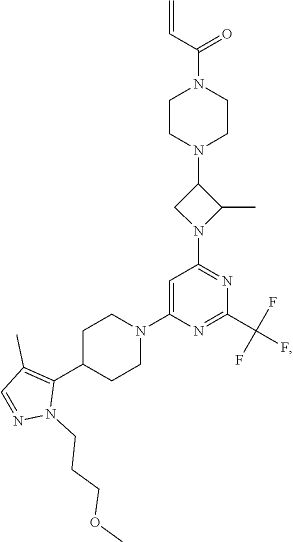 [SMILES: C=CC(=O)N1CCN(C2CN(c3cc(N4CCC(c5c(C)cnn5CCCOC)CC4)nc(C(F)(F)F)n3)C2C)CC1]

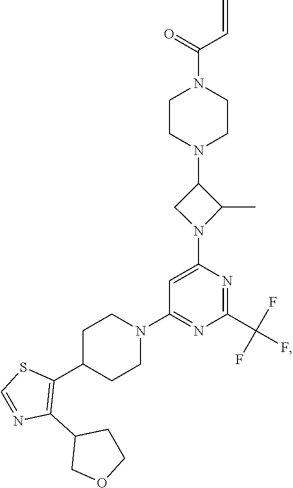 [SMILES: C=CC(=O)N1CCN(C2CN(c3cc(N4CCC(c5scnc5C5CCOC5)CC4)nc(C(F)(F)F)n3)C2C)CC1]

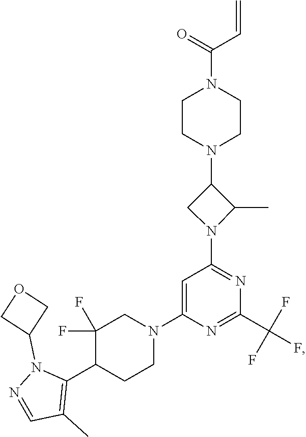 [SMILES: C=CC(=O)N1CCN(C2CN(c3cc(N4CCC(c5c(C)cnn5C5COC5)C(F)(F)C4)nc(C(F)(F)F)n3)C2C)CC1]

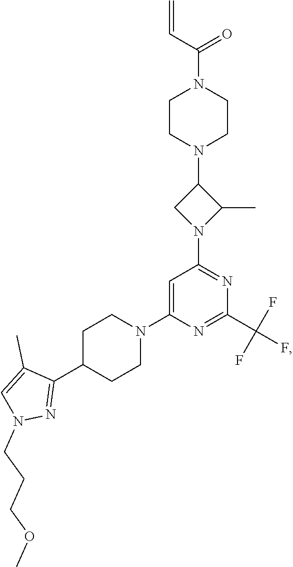 [SMILES: C=CC(=O)N1CCN(C2CN(c3cc(N4CCC(c5nn(CCCOC)cc5C)CC4)nc(C(F)(F)F)n3)C2C)CC1]

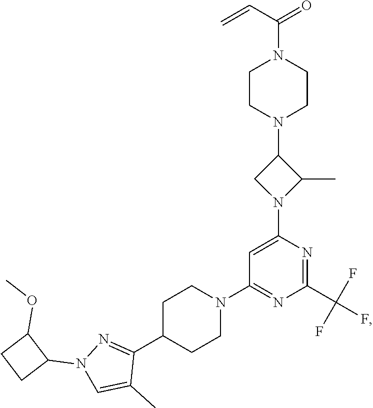 [SMILES: C=CC(=O)N1CCN(C2CN(c3cc(N4CCC(c5nn(C6CCC6OC)cc5C)CC4)nc(C(F)(F)F)n3)C2C)CC1]

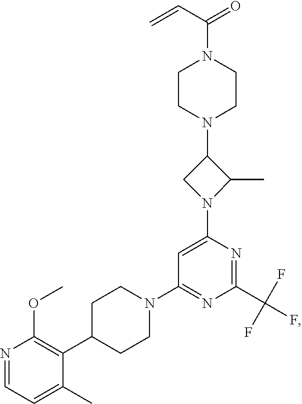 [SMILES: C=CC(=O)N1CCN(C2CN(c3cc(N4CCC(c5c(C)ccnc5OC)CC4)nc(C(F)(F)F)n3)C2C)CC1]

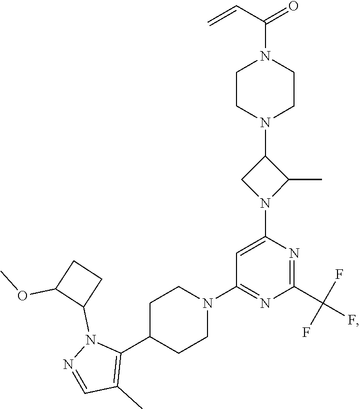 [SMILES: C=CC(=O)N1CCN(C2CN(c3cc(N4CCC(c5c(C)cnn5C5CCC5OC)CC4)nc(C(F)(F)F)n3)C2C)CC1]

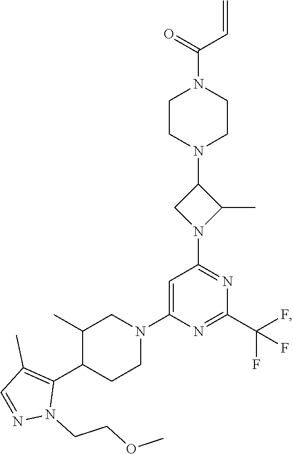 [SMILES: C=CC(=O)N1CCN(C2CN(c3cc(N4CCC(c5c(C)cnn5CCOC)C(C)C4)nc(C(F)(F)F)n3)C2C)CC1]

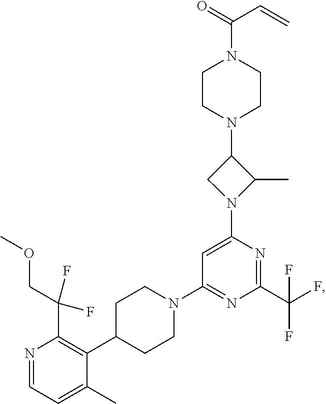 [SMILES: C=CC(=O)N1CCN(C2CN(c3cc(N4CCC(c5c(C)ccnc5C(F)(F)COC)CC4)nc(C(F)(F)F)n3)C2C)CC1]

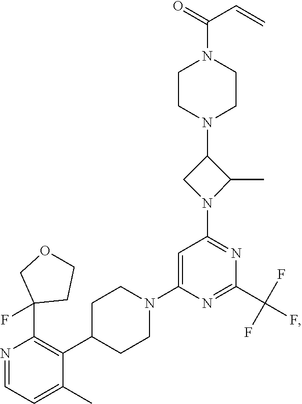 [SMILES: C=CC(=O)N1CCN(C2CN(c3cc(N4CCC(c5c(C)ccnc5C5(F)CCOC5)CC4)nc(C(F)(F)F)n3)C2C)CC1]

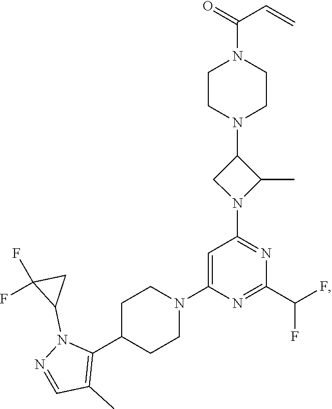 [SMILES: C=CC(=O)N1CCN(C2CN(c3cc(N4CCC(c5c(C)cnn5C5CC5(F)F)CC4)nc(C(F)F)n3)C2C)CC1]

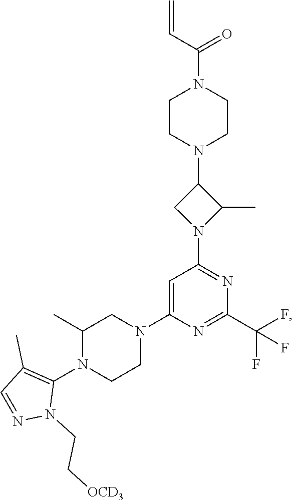

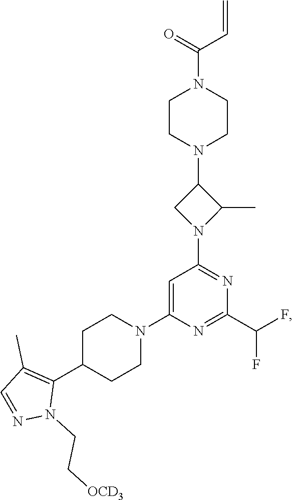

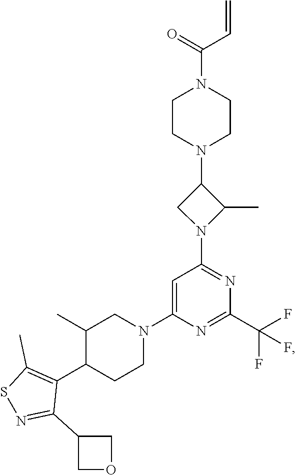 [SMILES: C=CC(=O)N1CCN(C2CN(c3cc(N4CCC(c5c(C6COC6)nsc5C)C(C)C4)nc(C(F)(F)F)n3)C2C)CC1]

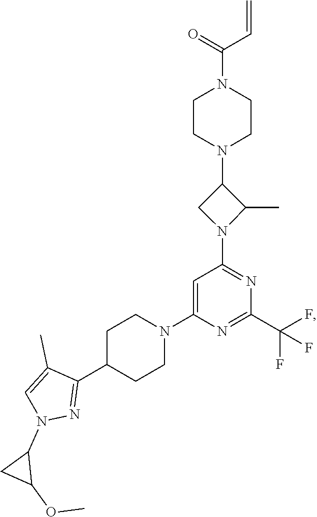 [SMILES: C=CC(=O)N1CCN(C2CN(c3cc(N4CCC(c5nn(C6CC6OC)cc5C)CC4)nc(C(F)(F)F)n3)C2C)CC1]

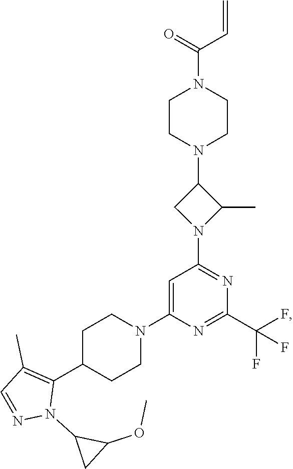 [SMILES: C=CC(=O)N1CCN(C2CN(c3cc(N4CCC(c5c(C)cnn5C5CC5OC)CC4)nc(C(F)(F)F)n3)C2C)CC1]

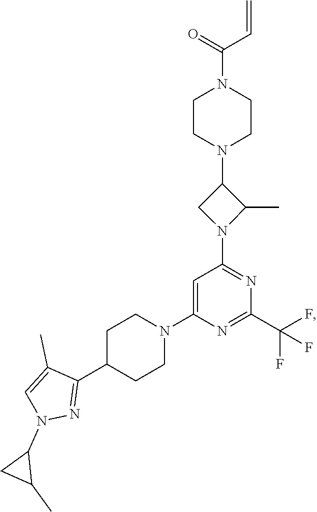 [SMILES: C=CC(=O)N1CCN(C2CN(c3cc(N4CCC(c5nn(C6CC6C)cc5C)CC4)nc(C(F)(F)F)n3)C2C)CC1]

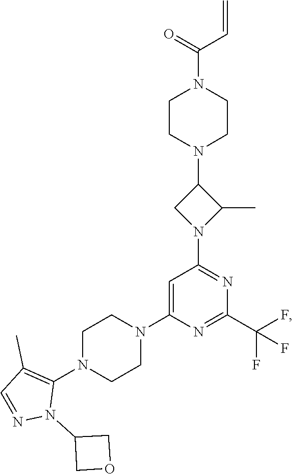 [SMILES: C=CC(=O)N1CCN(C2CN(c3cc(N4CCN(c5c(C)cnn5C5COC5)CC4)nc(C(F)(F)F)n3)C2C)CC1]

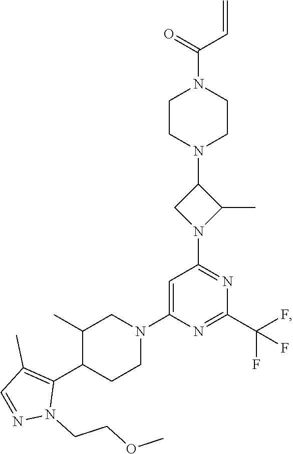 [SMILES: C=CC(=O)N1CCN(C2CN(c3cc(N4CCC(c5c(C)cnn5CCOC)C(C)C4)nc(C(F)(F)F)n3)C2C)CC1]

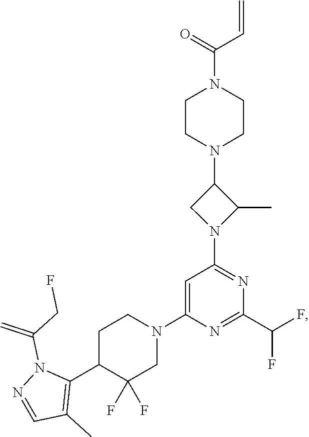 [SMILES: C=CC(=O)N1CCN(C2CN(c3cc(N4CCC(c5c(C)cnn5C(=C)CF)C(F)(F)C4)nc(C(F)F)n3)C2C)CC1]

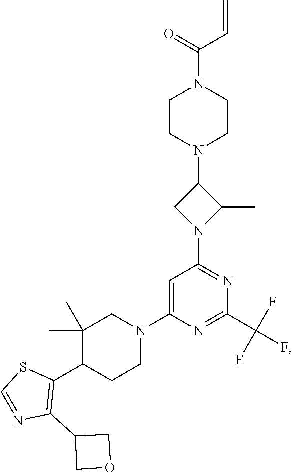 [SMILES: C=CC(=O)N1CCN(C2CN(c3cc(N4CCC(c5scnc5C5COC5)C(C)(C)C4)nc(C(F)(F)F)n3)C2C)CC1]

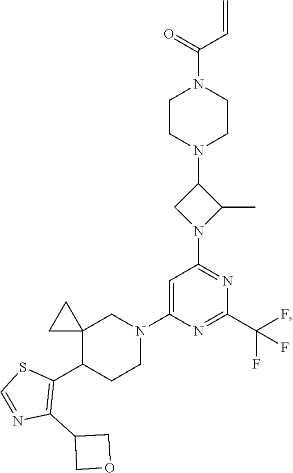 [SMILES: C=CC(=O)N1CCN(C2CN(c3cc(N4CCC(c5scnc5C5COC5)C5(CC5)C4)nc(C(F)(F)F)n3)C2C)CC1]

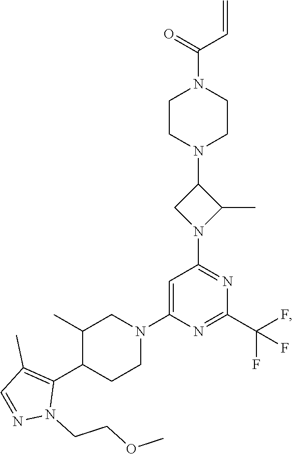 [SMILES: C=CC(=O)N1CCN(C2CN(c3cc(N4CCC(c5c(C)cnn5CCOC)C(C)C4)nc(C(F)(F)F)n3)C2C)CC1]

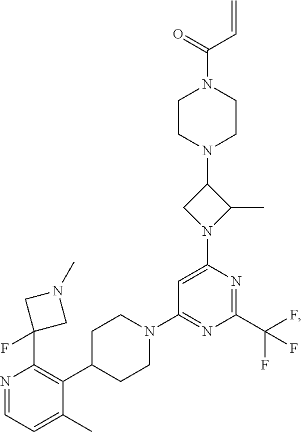 [SMILES: C=CC(=O)N1CCN(C2CN(c3cc(N4CCC(c5c(C)ccnc5C5(F)CN(C)C5)CC4)nc(C(F)(F)F)n3)C2C)CC1]

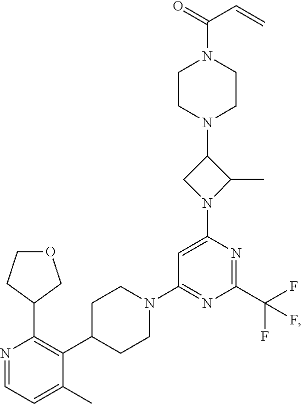 [SMILES: C=CC(=O)N1CCN(C2CN(c3cc(N4CCC(c5c(C)ccnc5C5CCOC5)CC4)nc(C(F)(F)F)n3)C2C)CC1]

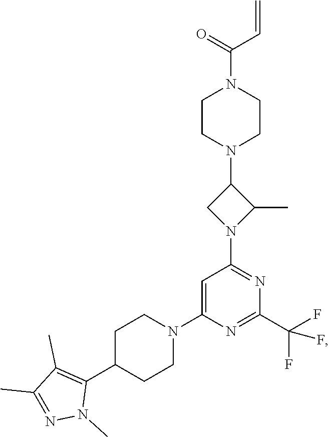 [SMILES: C=CC(=O)N1CCN(C2CN(c3cc(N4CCC(c5c(C)c(C)nn5C)CC4)nc(C(F)(F)F)n3)C2C)CC1]

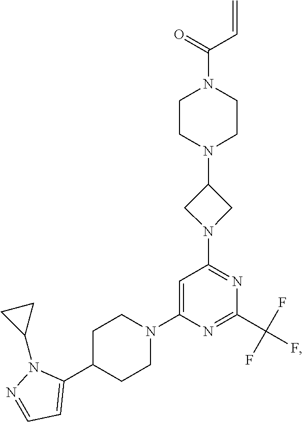 [SMILES: C=CC(=O)N1CCN(C2CN(c3cc(N4CCC(c5ccnn5C5CC5)CC4)nc(C(F)(F)F)n3)C2)CC1]

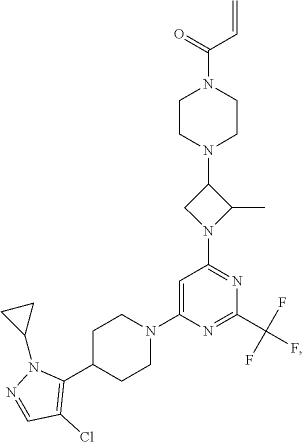 [SMILES: C=CC(=O)N1CCN(C2CN(c3cc(N4CCC(c5c(Cl)cnn5C5CC5)CC4)nc(C(F)(F)F)n3)C2C)CC1]

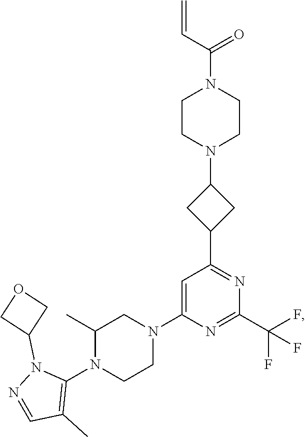 [SMILES: C=CC(=O)N1CCN(C2CC(c3cc(N4CCN(c5c(C)cnn5C5COC5)C(C)C4)nc(C(F)(F)F)n3)C2)CC1]

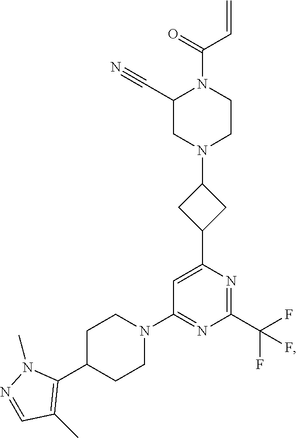 [SMILES: C=CC(=O)N1CCN(C2CC(c3cc(N4CCC(c5c(C)cnn5C)CC4)nc(C(F)(F)F)n3)C2)CC1C#N]

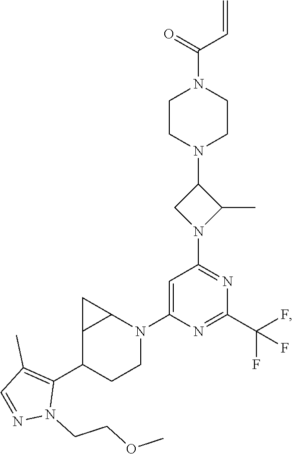 [SMILES: C=CC(=O)N1CCN(C2CN(c3cc(N4CCC(c5c(C)cnn5CCOC)C5CC54)nc(C(F)(F)F)n3)C2C)CC1]

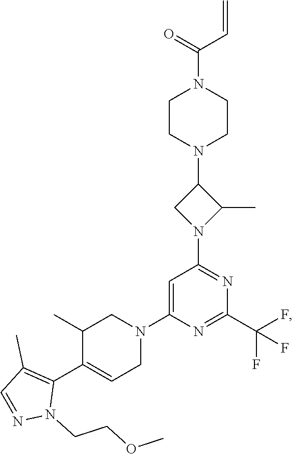 [SMILES: C=CC(=O)N1CCN(C2CN(c3cc(N4CC=C(c5c(C)cnn5CCOC)C(C)C4)nc(C(F)(F)F)n3)C2C)CC1]

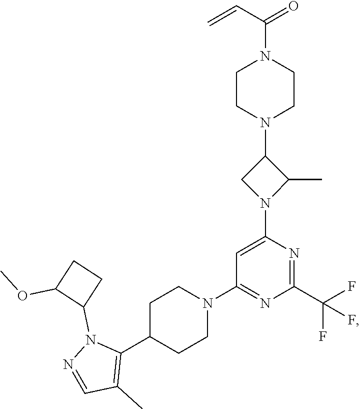 [SMILES: C=CC(=O)N1CCN(C2CN(c3cc(N4CCC(c5c(C)cnn5C5CCC5OC)CC4)nc(C(F)(F)F)n3)C2C)CC1]

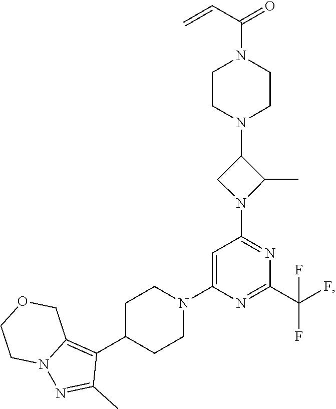 [SMILES: C=CC(=O)N1CCN(C2CN(c3cc(N4CCC(c5c(C)nn6c5COCC6)CC4)nc(C(F)(F)F)n3)C2C)CC1]

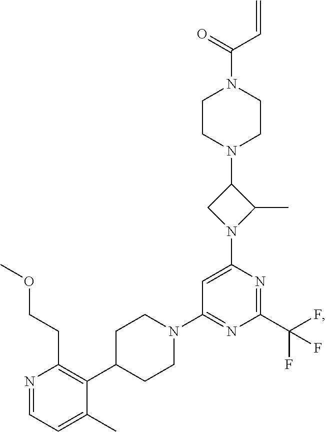 [SMILES: C=CC(=O)N1CCN(C2CN(c3cc(N4CCC(c5c(C)ccnc5CCOC)CC4)nc(C(F)(F)F)n3)C2C)CC1]

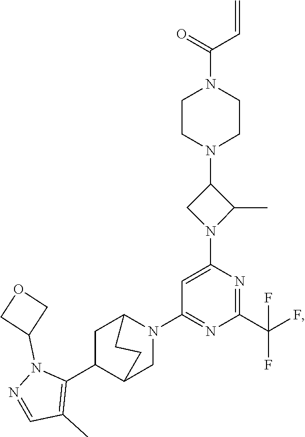 [SMILES: C=CC(=O)N1CCN(C2CN(c3cc(N4CC5CCC4CC5c4c(C)cnn4C4COC4)nc(C(F)(F)F)n3)C2C)CC1]

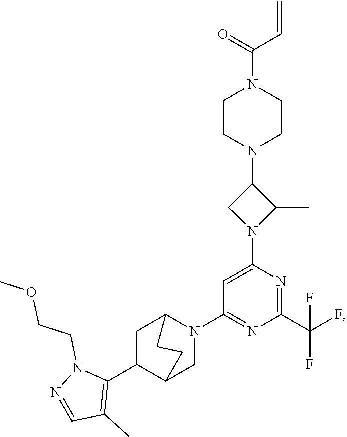 [SMILES: C=CC(=O)N1CCN(C2CN(c3cc(N4CC5CCC4CC5c4c(C)cnn4CCOC)nc(C(F)(F)F)n3)C2C)CC1]

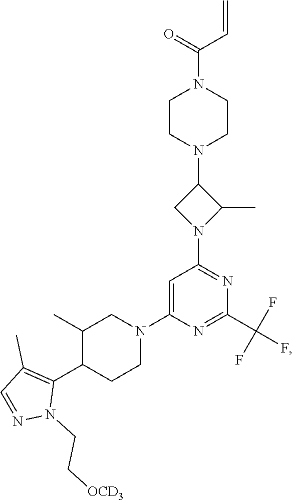

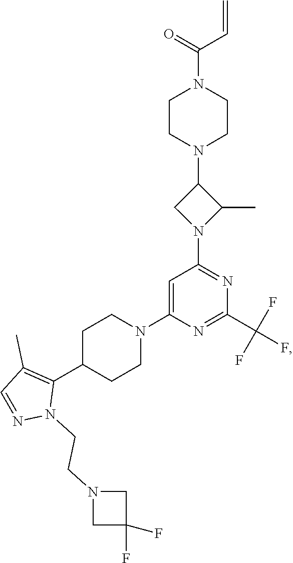 [SMILES: C=CC(=O)N1CCN(C2CN(c3cc(N4CCC(c5c(C)cnn5CCN5CC(F)(F)C5)CC4)nc(C(F)(F)F)n3)C2C)CC1]

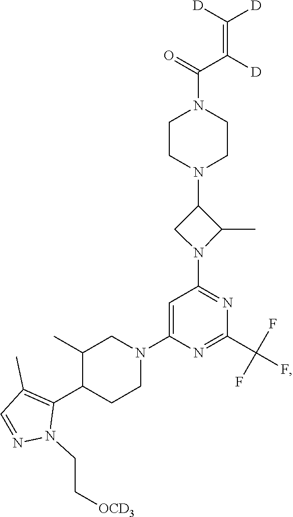

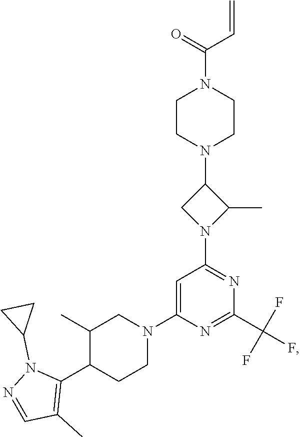 [SMILES: C=CC(=O)N1CCN(C2CN(c3cc(N4CCC(c5c(C)cnn5C5CC5)C(C)C4)nc(C(F)(F)F)n3)C2C)CC1]

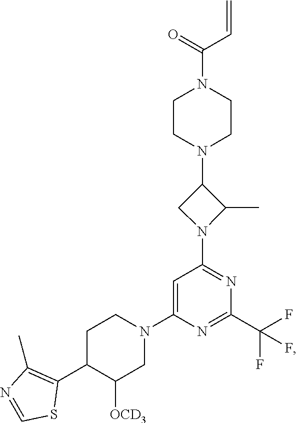

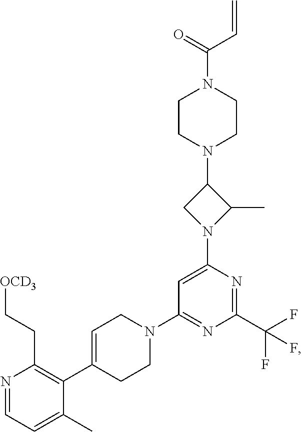

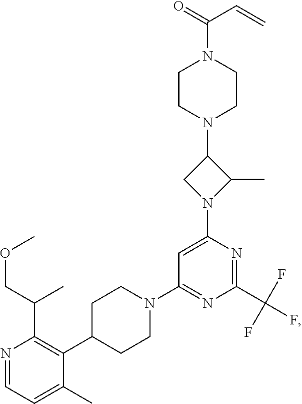 [SMILES: C=CC(=O)N1CCN(C2CN(c3cc(N4CCC(c5c(C)ccnc5C(C)COC)CC4)nc(C(F)(F)F)n3)C2C)CC1]

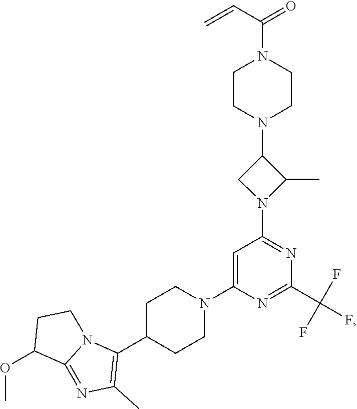 [SMILES: C=CC(=O)N1CCN(C2CN(c3cc(N4CCC(c5c(C)nc6n5CCC6OC)CC4)nc(C(F)(F)F)n3)C2C)CC1]

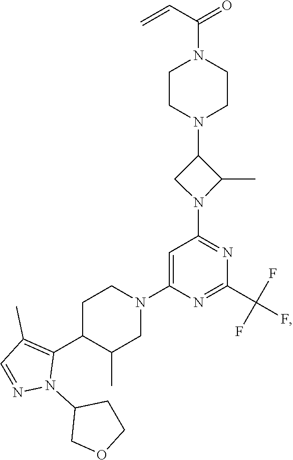 [SMILES: C=CC(=O)N1CCN(C2CN(c3cc(N4CCC(c5c(C)cnn5C5CCOC5)C(C)C4)nc(C(F)(F)F)n3)C2C)CC1]

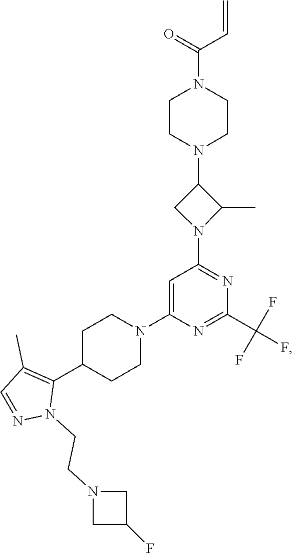 [SMILES: C=CC(=O)N1CCN(C2CN(c3cc(N4CCC(c5c(C)cnn5CCN5CC(F)C5)CC4)nc(C(F)(F)F)n3)C2C)CC1]

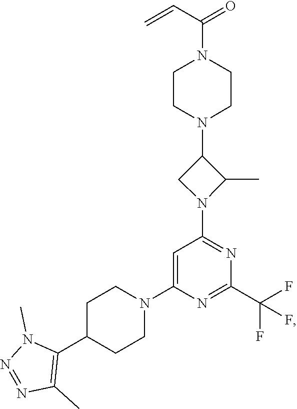 [SMILES: C=CC(=O)N1CCN(C2CN(c3cc(N4CCC(c5c(C)nnn5C)CC4)nc(C(F)(F)F)n3)C2C)CC1]

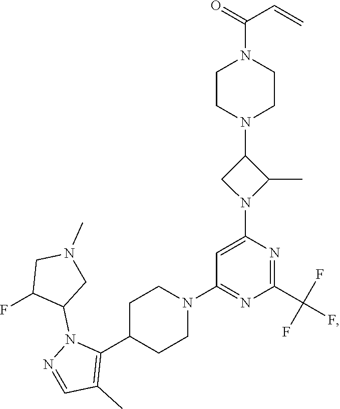 [SMILES: C=CC(=O)N1CCN(C2CN(c3cc(N4CCC(c5c(C)cnn5C5CN(C)CC5F)CC4)nc(C(F)(F)F)n3)C2C)CC1]

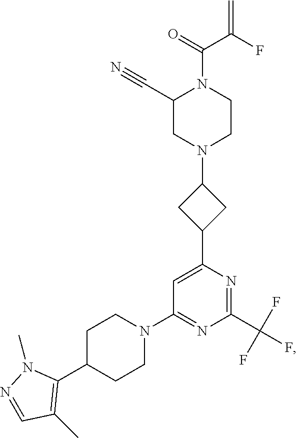 [SMILES: C=C(F)C(=O)N1CCN(C2CC(c3cc(N4CCC(c5c(C)cnn5C)CC4)nc(C(F)(F)F)n3)C2)CC1C#N]

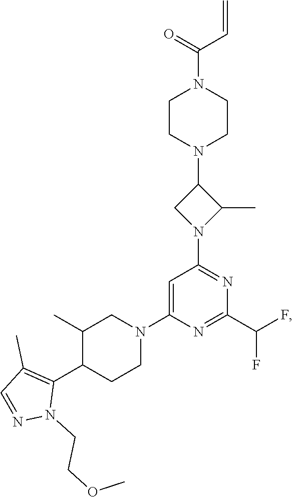 [SMILES: C=CC(=O)N1CCN(C2CN(c3cc(N4CCC(c5c(C)cnn5CCOC)C(C)C4)nc(C(F)F)n3)C2C)CC1]

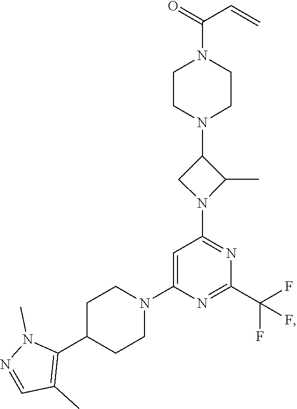 [SMILES: C=CC(=O)N1CCN(C2CN(c3cc(N4CCC(c5c(C)cnn5C)CC4)nc(C(F)(F)F)n3)C2C)CC1]

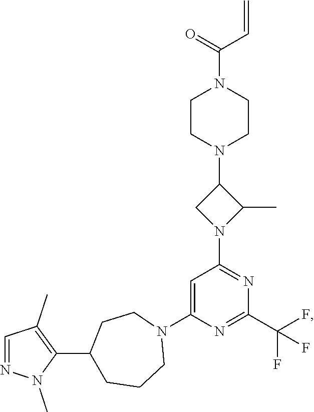 [SMILES: C=CC(=O)N1CCN(C2CN(c3cc(N4CCCC(c5c(C)cnn5C)CC4)nc(C(F)(F)F)n3)C2C)CC1]

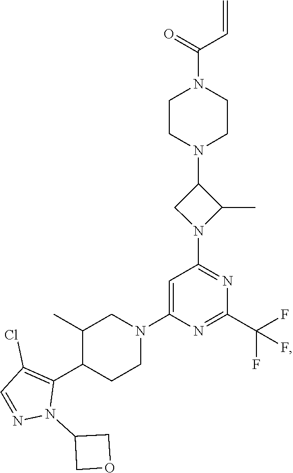 [SMILES: C=CC(=O)N1CCN(C2CN(c3cc(N4CCC(c5c(Cl)cnn5C5COC5)C(C)C4)nc(C(F)(F)F)n3)C2C)CC1]

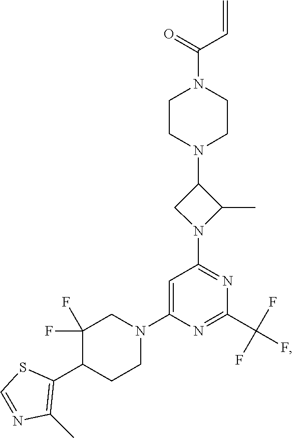 [SMILES: C=CC(=O)N1CCN(C2CN(c3cc(N4CCC(c5scnc5C)C(F)(F)C4)nc(C(F)(F)F)n3)C2C)CC1]

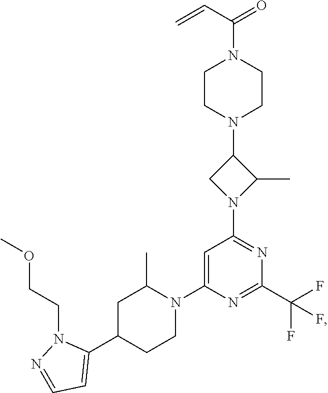 [SMILES: C=CC(=O)N1CCN(C2CN(c3cc(N4CCC(c5ccnn5CCOC)CC4C)nc(C(F)(F)F)n3)C2C)CC1]

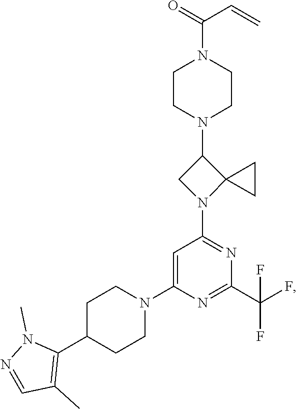 [SMILES: C=CC(=O)N1CCN(C2CN(c3cc(N4CCC(c5c(C)cnn5C)CC4)nc(C(F)(F)F)n3)C23CC3)CC1]

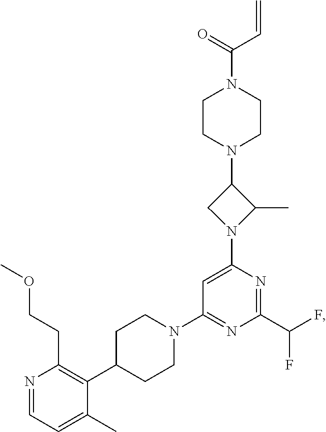 [SMILES: C=CC(=O)N1CCN(C2CN(c3cc(N4CCC(c5c(C)ccnc5CCOC)CC4)nc(C(F)F)n3)C2C)CC1]

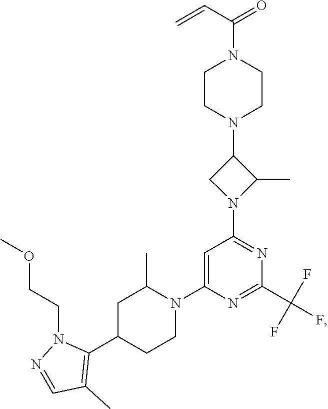 [SMILES: C=CC(=O)N1CCN(C2CN(c3cc(N4CCC(c5c(C)cnn5CCOC)CC4C)nc(C(F)(F)F)n3)C2C)CC1]

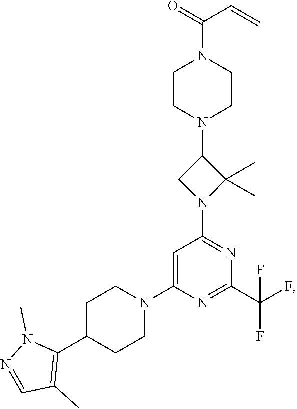 [SMILES: C=CC(=O)N1CCN(C2CN(c3cc(N4CCC(c5c(C)cnn5C)CC4)nc(C(F)(F)F)n3)C2(C)C)CC1]

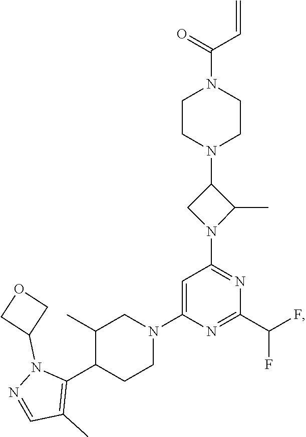 [SMILES: C=CC(=O)N1CCN(C2CN(c3cc(N4CCC(c5c(C)cnn5C5COC5)C(C)C4)nc(C(F)F)n3)C2C)CC1]

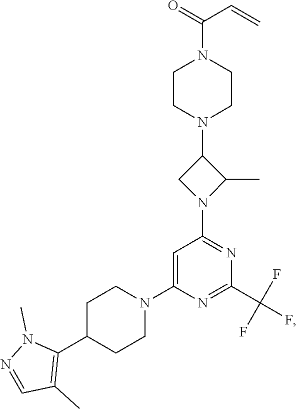 [SMILES: C=CC(=O)N1CCN(C2CN(c3cc(N4CCC(c5c(C)cnn5C)CC4)nc(C(F)(F)F)n3)C2C)CC1]

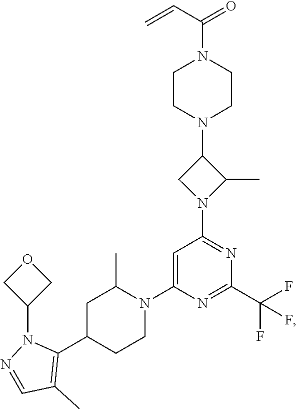 [SMILES: C=CC(=O)N1CCN(C2CN(c3cc(N4CCC(c5c(C)cnn5C5COC5)CC4C)nc(C(F)(F)F)n3)C2C)CC1]

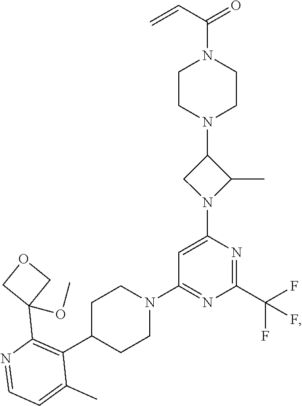 [SMILES: C=CC(=O)N1CCN(C2CN(c3cc(N4CCC(c5c(C)ccnc5C5(OC)COC5)CC4)nc(C(F)(F)F)n3)C2C)CC1]

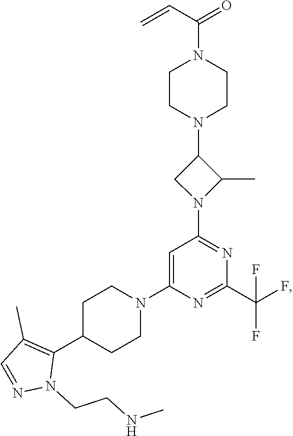 [SMILES: C=CC(=O)N1CCN(C2CN(c3cc(N4CCC(c5c(C)cnn5CCNC)CC4)nc(C(F)(F)F)n3)C2C)CC1]

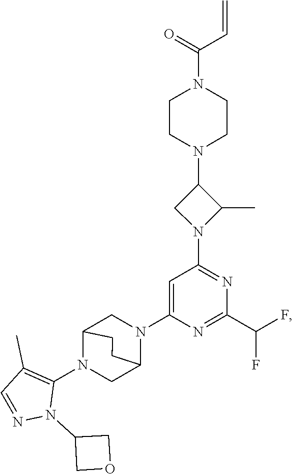 [SMILES: C=CC(=O)N1CCN(C2CN(c3cc(N4CC5CCC4CN5c4c(C)cnn4C4COC4)nc(C(F)F)n3)C2C)CC1]

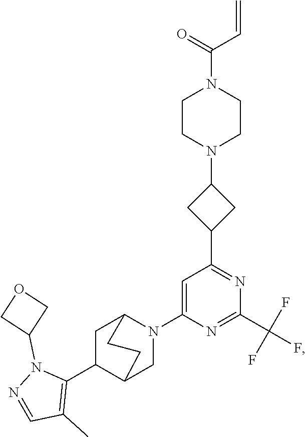 [SMILES: C=CC(=O)N1CCN(C2CC(c3cc(N4CC5CCC4CC5c4c(C)cnn4C4COC4)nc(C(F)(F)F)n3)C2)CC1]

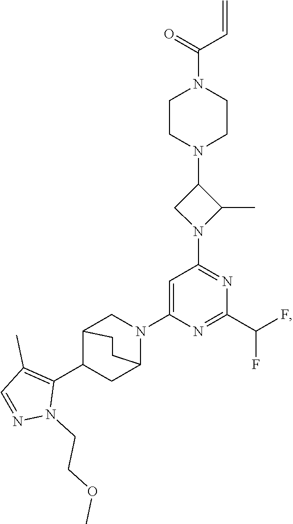 [SMILES: C=CC(=O)N1CCN(C2CN(c3cc(N4CC5CCC4CC5c4c(C)cnn4CCOC)nc(C(F)F)n3)C2C)CC1]

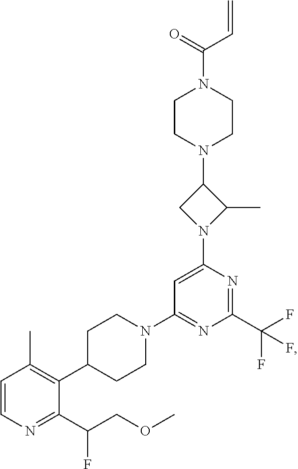 [SMILES: C=CC(=O)N1CCN(C2CN(c3cc(N4CCC(c5c(C)ccnc5C(F)COC)CC4)nc(C(F)(F)F)n3)C2C)CC1]

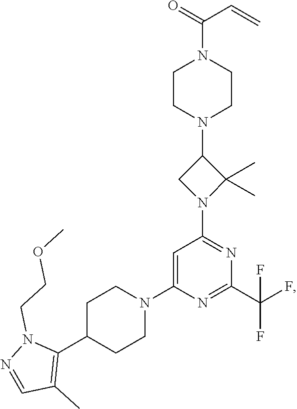 [SMILES: C=CC(=O)N1CCN(C2CN(c3cc(N4CCC(c5c(C)cnn5CCOC)CC4)nc(C(F)(F)F)n3)C2(C)C)CC1]

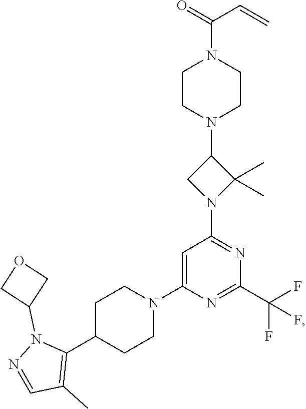 [SMILES: C=CC(=O)N1CCN(C2CN(c3cc(N4CCC(c5c(C)cnn5C5COC5)CC4)nc(C(F)(F)F)n3)C2(C)C)CC1]

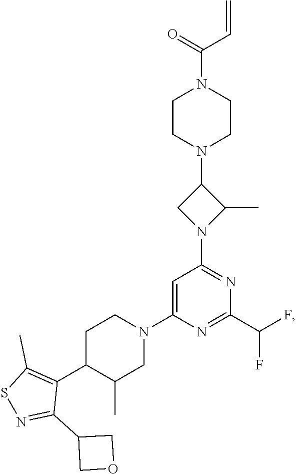 [SMILES: C=CC(=O)N1CCN(C2CN(c3cc(N4CCC(c5c(C6COC6)nsc5C)C(C)C4)nc(C(F)F)n3)C2C)CC1]

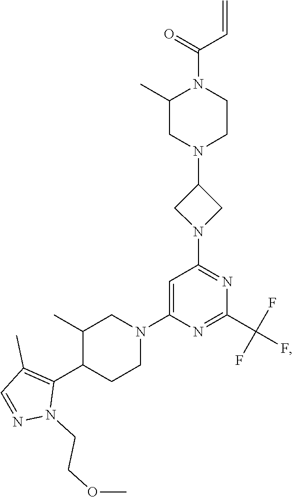 [SMILES: C=CC(=O)N1CCN(C2CN(c3cc(N4CCC(c5c(C)cnn5CCOC)C(C)C4)nc(C(F)(F)F)n3)C2)CC1C]

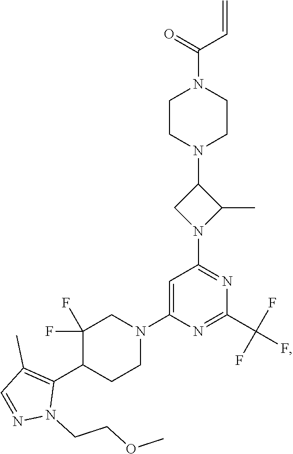 [SMILES: C=CC(=O)N1CCN(C2CN(c3cc(N4CCC(c5c(C)cnn5CCOC)C(F)(F)C4)nc(C(F)(F)F)n3)C2C)CC1]

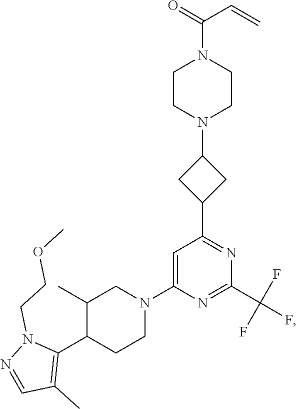 [SMILES: C=CC(=O)N1CCN(C2CC(c3cc(N4CCC(c5c(C)cnn5CCOC)C(C)C4)nc(C(F)(F)F)n3)C2)CC1]

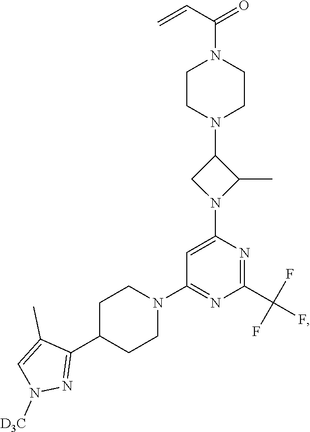 [SMILES: [2H]C([2H])([2H])n1cc(C)c(C2CCN(c3cc(N4CC(N5CCN(C(=O)C=C)CC5)C4C)nc(C(F)(F)F)n3)CC2)n1]

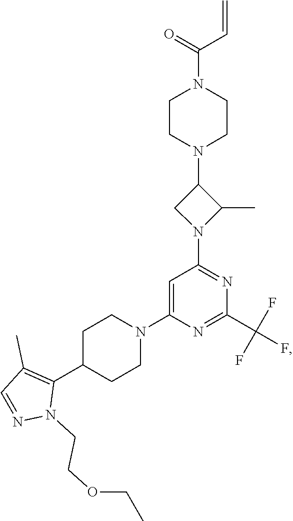 [SMILES: C=CC(=O)N1CCN(C2CN(c3cc(N4CCC(c5c(C)cnn5CCOCC)CC4)nc(C(F)(F)F)n3)C2C)CC1]

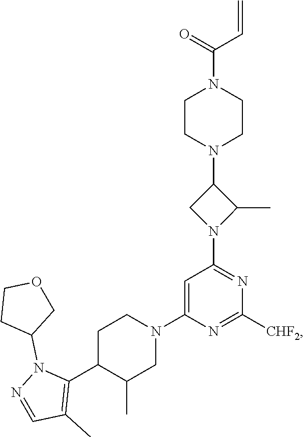 [SMILES: C=CC(=O)N1CCN(C2CN(c3cc(N4CCC(c5c(C)cnn5C5CCOC5)C(C)C4)nc(C(F)F)n3)C2C)CC1]

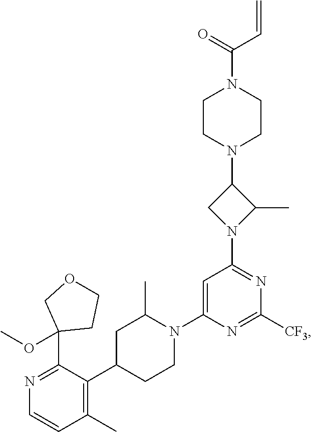 [SMILES: C=CC(=O)N1CCN(C2CN(c3cc(N4CCC(c5c(C)ccnc5C5(OC)CCOC5)CC4C)nc(C(F)(F)F)n3)C2C)CC1]

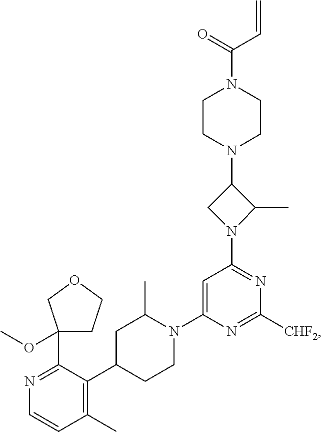 [SMILES: C=CC(=O)N1CCN(C2CN(c3cc(N4CCC(c5c(C)ccnc5C5(OC)CCOC5)CC4C)nc(C(F)F)n3)C2C)CC1]

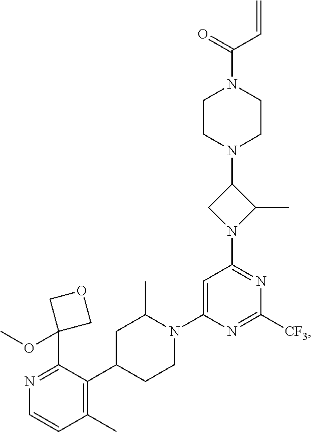 [SMILES: C=CC(=O)N1CCN(C2CN(c3cc(N4CCC(c5c(C)ccnc5C5(OC)COC5)CC4C)nc(C(F)(F)F)n3)C2C)CC1]

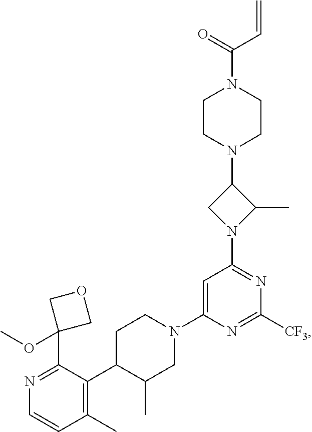 [SMILES: C=CC(=O)N1CCN(C2CN(c3cc(N4CCC(c5c(C)ccnc5C5(OC)COC5)C(C)C4)nc(C(F)(F)F)n3)C2C)CC1]

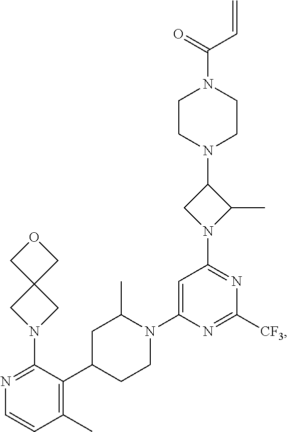 [SMILES: C=CC(=O)N1CCN(C2CN(c3cc(N4CCC(c5c(C)ccnc5N5CC6(COC6)C5)CC4C)nc(C(F)(F)F)n3)C2C)CC1]

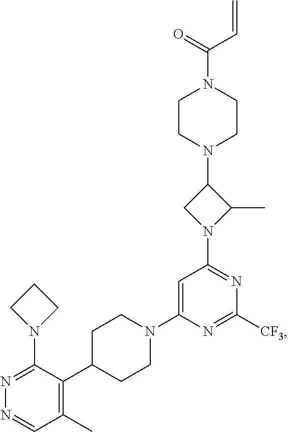 [SMILES: C=CC(=O)N1CCN(C2CN(c3cc(N4CCC(c5c(C)cnnc5N5CCC5)CC4)nc(C(F)(F)F)n3)C2C)CC1]

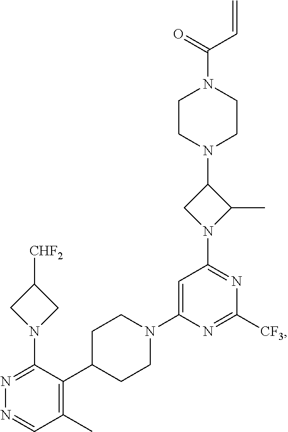 [SMILES: C=CC(=O)N1CCN(C2CN(c3cc(N4CCC(c5c(C)cnnc5N5CC(C(F)F)C5)CC4)nc(C(F)(F)F)n3)C2C)CC1]

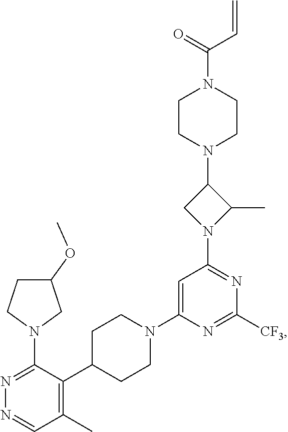 [SMILES: C=CC(=O)N1CCN(C2CN(c3cc(N4CCC(c5c(C)cnnc5N5CCC(OC)C5)CC4)nc(C(F)(F)F)n3)C2C)CC1]

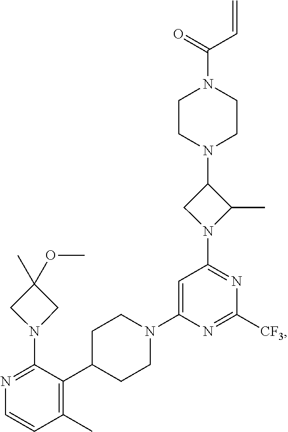 [SMILES: C=CC(=O)N1CCN(C2CN(c3cc(N4CCC(c5c(C)ccnc5N5CC(C)(OC)C5)CC4)nc(C(F)(F)F)n3)C2C)CC1]

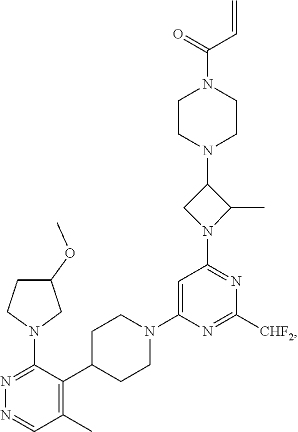 [SMILES: C=CC(=O)N1CCN(C2CN(c3cc(N4CCC(c5c(C)cnnc5N5CCC(OC)C5)CC4)nc(C(F)F)n3)C2C)CC1]

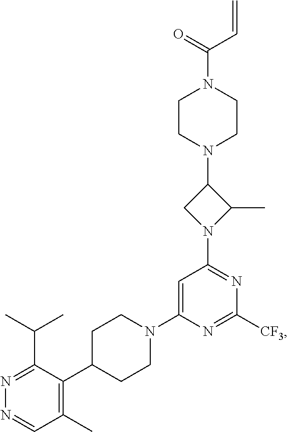 [SMILES: C=CC(=O)N1CCN(C2CN(c3cc(N4CCC(c5c(C)cnnc5C(C)C)CC4)nc(C(F)(F)F)n3)C2C)CC1]

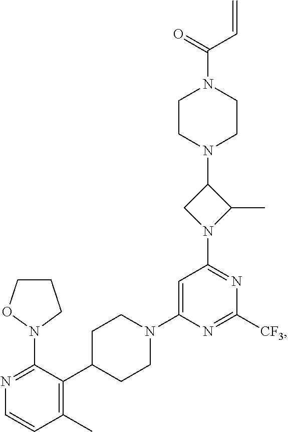 [SMILES: C=CC(=O)N1CCN(C2CN(c3cc(N4CCC(c5c(C)ccnc5N5CCCO5)CC4)nc(C(F)(F)F)n3)C2C)CC1]

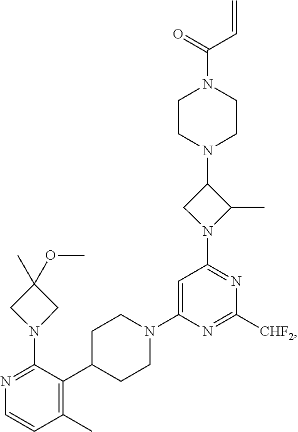 [SMILES: C=CC(=O)N1CCN(C2CN(c3cc(N4CCC(c5c(C)ccnc5N5CC(C)(OC)C5)CC4)nc(C(F)F)n3)C2C)CC1]

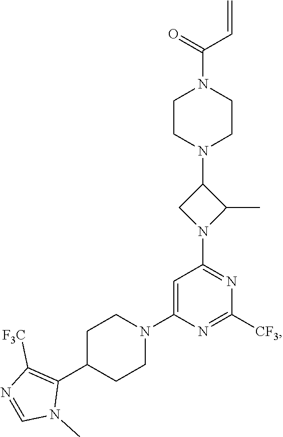 [SMILES: C=CC(=O)N1CCN(C2CN(c3cc(N4CCC(c5c(C(F)(F)F)ncn5C)CC4)nc(C(F)(F)F)n3)C2C)CC1]

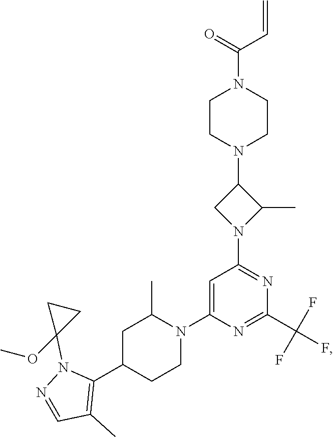 [SMILES: C=CC(=O)N1CCN(C2CN(c3cc(N4CCC(c5c(C)cnn5C5(OC)CC5)CC4C)nc(C(F)(F)F)n3)C2C)CC1]

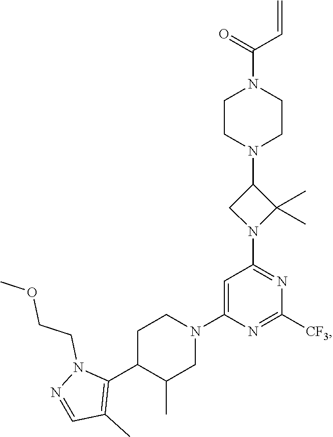 [SMILES: C=CC(=O)N1CCN(C2CN(c3cc(N4CCC(c5c(C)cnn5CCOC)C(C)C4)nc(C(F)(F)F)n3)C2(C)C)CC1]

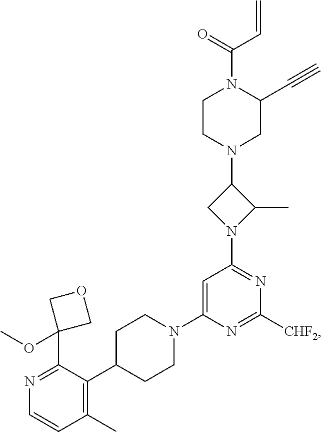 [SMILES: C#CC1CN(C2CN(c3cc(N4CCC(c5c(C)ccnc5C5(OC)COC5)CC4)nc(C(F)F)n3)C2C)CCN1C(=O)C=C]

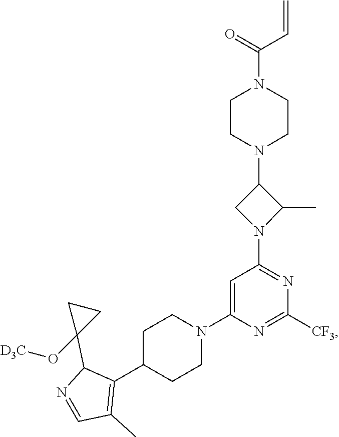 [SMILES: [2H]C([2H])([2H])OC1(C2N=CC(C)=C2C2CCN(c3cc(N4CC(N5CCN(C(=O)C=C)CC5)C4C)nc(C(F)(F)F)n3)CC2)CC1]

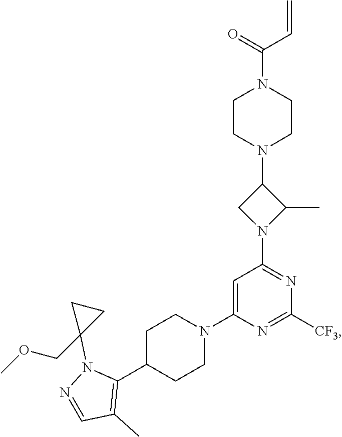 [SMILES: C=CC(=O)N1CCN(C2CN(c3cc(N4CCC(c5c(C)cnn5C5(COC)CC5)CC4)nc(C(F)(F)F)n3)C2C)CC1]

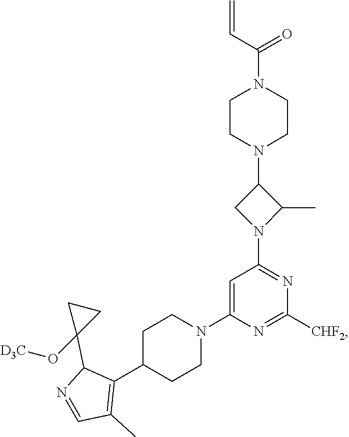 [SMILES: [2H]C([2H])([2H])OC1(C2N=CC(C)=C2C2CCN(c3cc(N4CC(N5CCN(C(=O)C=C)CC5)C4C)nc(C(F)F)n3)CC2)CC1]

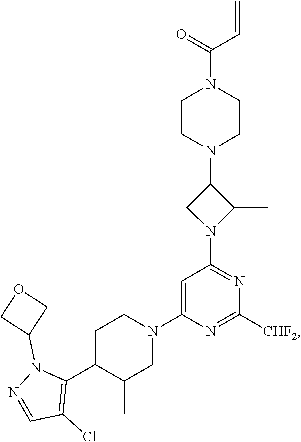 [SMILES: C=CC(=O)N1CCN(C2CN(c3cc(N4CCC(c5c(Cl)cnn5C5COC5)C(C)C4)nc(C(F)F)n3)C2C)CC1]

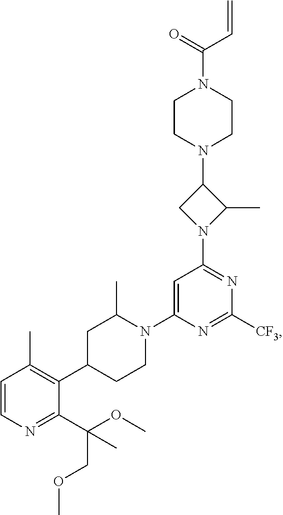 [SMILES: C=CC(=O)N1CCN(C2CN(c3cc(N4CCC(c5c(C)ccnc5C(C)(COC)OC)CC4C)nc(C(F)(F)F)n3)C2C)CC1]

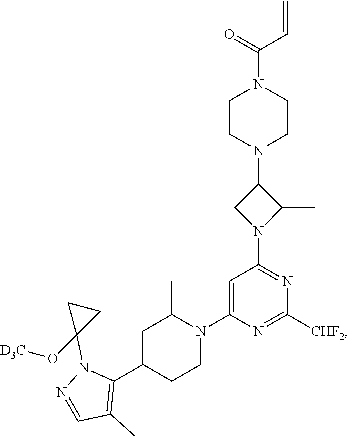 [SMILES: [2H]C([2H])([2H])OC1(n2ncc(C)c2C2CCN(c3cc(N4CC(N5CCN(C(=O)C=C)CC5)C4C)nc(C(F)F)n3)C(C)C2)CC1]

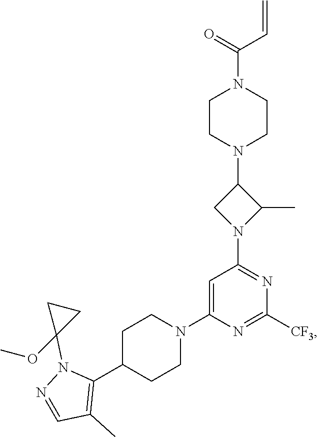 [SMILES: C=CC(=O)N1CCN(C2CN(c3cc(N4CCC(c5c(C)cnn5C5(OC)CC5)CC4)nc(C(F)(F)F)n3)C2C)CC1]

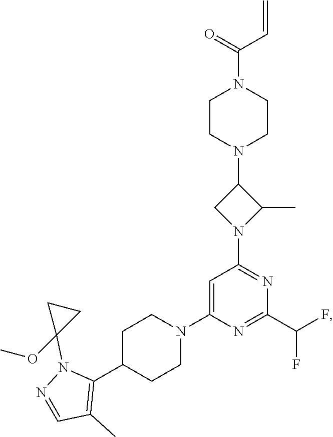 [SMILES: C=CC(=O)N1CCN(C2CN(c3cc(N4CCC(c5c(C)cnn5C5(OC)CC5)CC4)nc(C(F)F)n3)C2C)CC1]

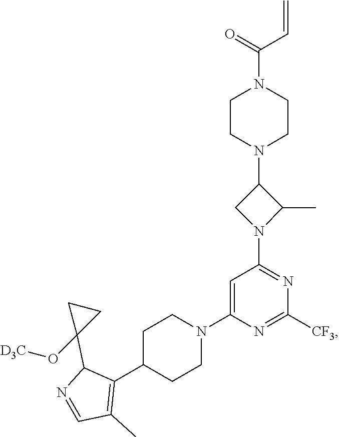 [SMILES: [2H]C([2H])([2H])OC1(C2N=CC(C)=C2C2CCN(c3cc(N4CC(N5CCN(C(=O)C=C)CC5)C4C)nc(C(F)(F)F)n3)CC2)CC1]

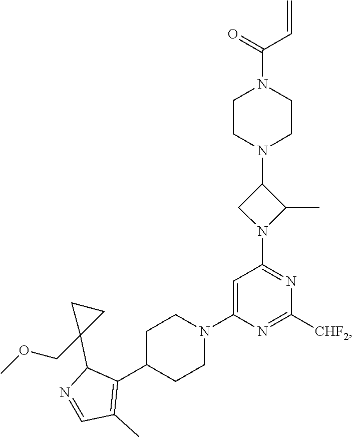 [SMILES: C=CC(=O)N1CCN(C2CN(c3cc(N4CCC(C5=C(C)C=NC5C5(COC)CC5)CC4)nc(C(F)F)n3)C2C)CC1]

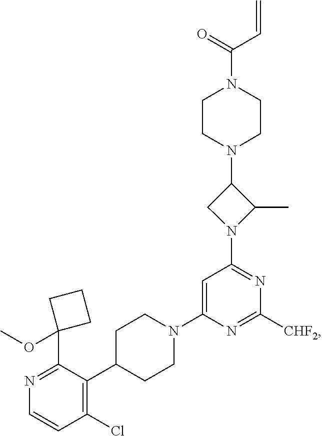 [SMILES: C=CC(=O)N1CCN(C2CN(c3cc(N4CCC(c5c(Cl)ccnc5C5(OC)CCC5)CC4)nc(C(F)F)n3)C2C)CC1]

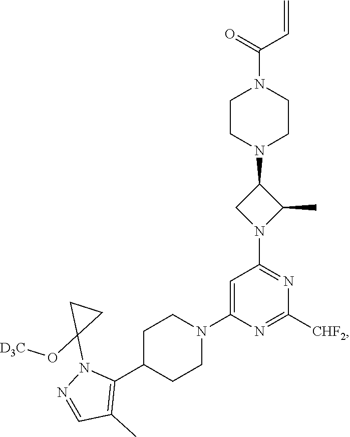 [SMILES: [2H]C([2H])([2H])OC1(n2ncc(C)c2C2CCN(c3cc(N4C[C@@H](N5CCN(C(=O)C=C)CC5)[C@H]4C)nc(C(F)F)n3)CC2)CC1]

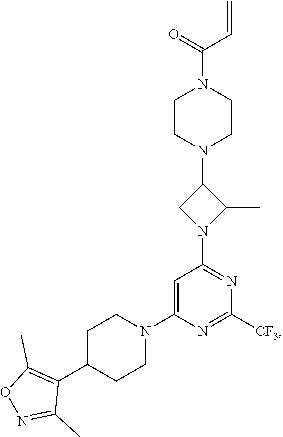 [SMILES: C=CC(=O)N1CCN(C2CN(c3cc(N4CCC(c5c(C)noc5C)CC4)nc(C(F)(F)F)n3)C2C)CC1]

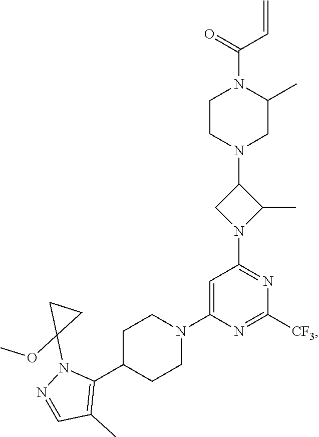 [SMILES: C=CC(=O)N1CCN(C2CN(c3cc(N4CCC(c5c(C)cnn5C5(OC)CC5)CC4)nc(C(F)(F)F)n3)C2C)CC1C]

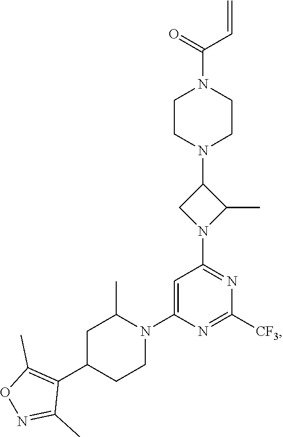 [SMILES: C=CC(=O)N1CCN(C2CN(c3cc(N4CCC(c5c(C)noc5C)CC4C)nc(C(F)(F)F)n3)C2C)CC1]

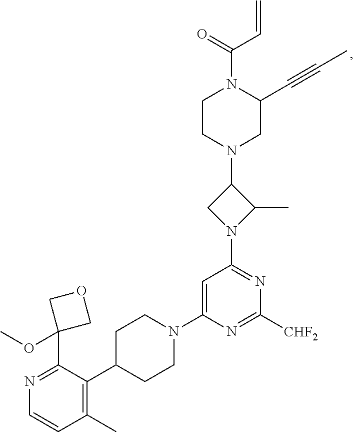 [SMILES: C=CC(=O)N1CCN(C2CN(c3cc(N4CCC(c5c(C)ccnc5C5(OC)COC5)CC4)nc(C(F)F)n3)C2C)CC1C#CC]

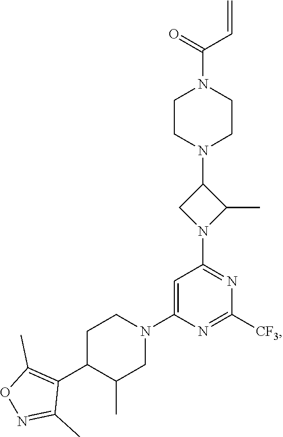 [SMILES: C=CC(=O)N1CCN(C2CN(c3cc(N4CCC(c5c(C)noc5C)C(C)C4)nc(C(F)(F)F)n3)C2C)CC1]

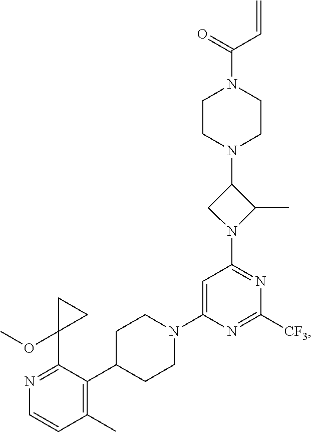 [SMILES: C=CC(=O)N1CCN(C2CN(c3cc(N4CCC(c5c(C)ccnc5C5(OC)CC5)CC4)nc(C(F)(F)F)n3)C2C)CC1]

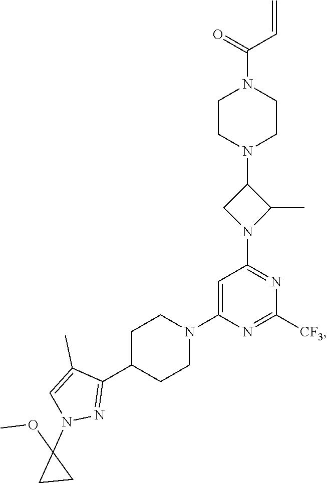 [SMILES: C=CC(=O)N1CCN(C2CN(c3cc(N4CCC(c5nn(C6(OC)CC6)cc5C)CC4)nc(C(F)(F)F)n3)C2C)CC1]

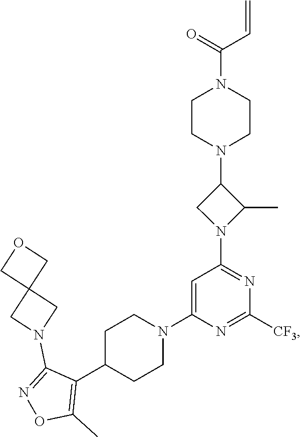 [SMILES: C=CC(=O)N1CCN(C2CN(c3cc(N4CCC(c5c(N6CC7(COC7)C6)noc5C)CC4)nc(C(F)(F)F)n3)C2C)CC1]

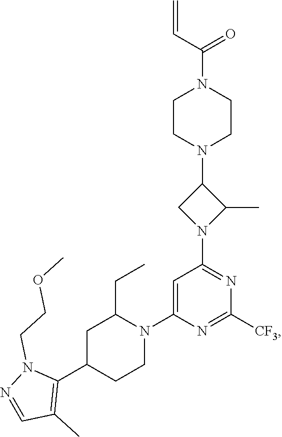 [SMILES: C=CC(=O)N1CCN(C2CN(c3cc(N4CCC(c5c(C)cnn5CCOC)CC4CC)nc(C(F)(F)F)n3)C2C)CC1]

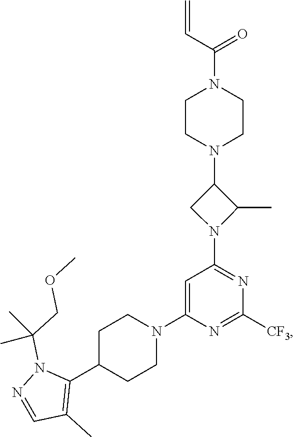 [SMILES: C=CC(=O)N1CCN(C2CN(c3cc(N4CCC(c5c(C)cnn5C(C)(C)COC)CC4)nc(C(F)(F)F)n3)C2C)CC1]

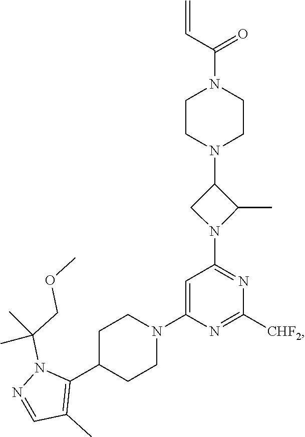 [SMILES: C=CC(=O)N1CCN(C2CN(c3cc(N4CCC(c5c(C)cnn5C(C)(C)COC)CC4)nc(C(F)F)n3)C2C)CC1]

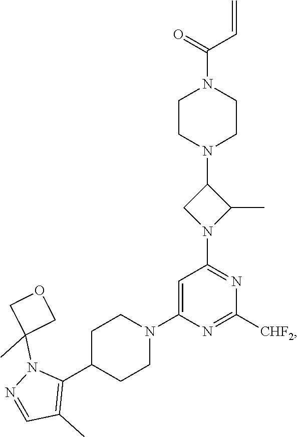 [SMILES: C=CC(=O)N1CCN(C2CN(c3cc(N4CCC(c5c(C)cnn5C5(C)COC5)CC4)nc(C(F)F)n3)C2C)CC1]

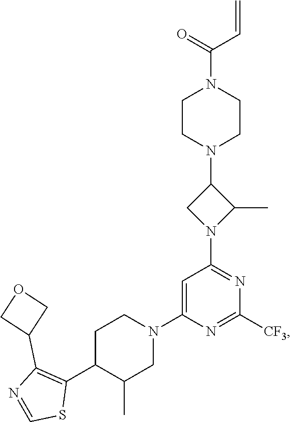 [SMILES: C=CC(=O)N1CCN(C2CN(c3cc(N4CCC(c5scnc5C5COC5)C(C)C4)nc(C(F)(F)F)n3)C2C)CC1]

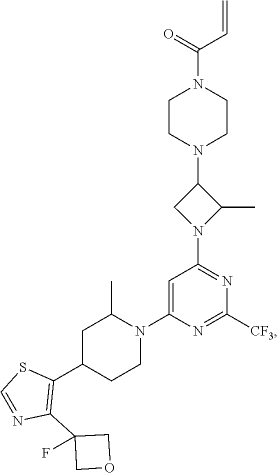 [SMILES: C=CC(=O)N1CCN(C2CN(c3cc(N4CCC(c5scnc5C5(F)COC5)CC4C)nc(C(F)(F)F)n3)C2C)CC1]

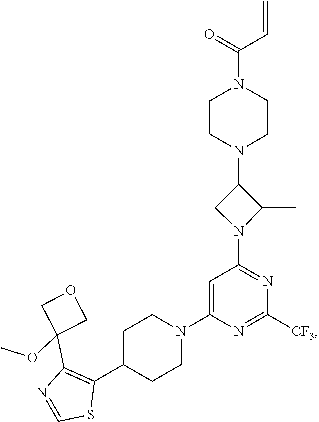 [SMILES: C=CC(=O)N1CCN(C2CN(c3cc(N4CCC(c5scnc5C5(OC)COC5)CC4)nc(C(F)(F)F)n3)C2C)CC1]

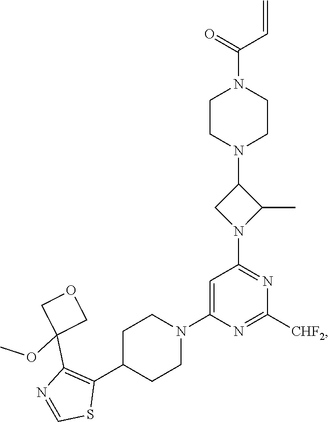 [SMILES: C=CC(=O)N1CCN(C2CN(c3cc(N4CCC(c5scnc5C5(OC)COC5)CC4)nc(C(F)F)n3)C2C)CC1]

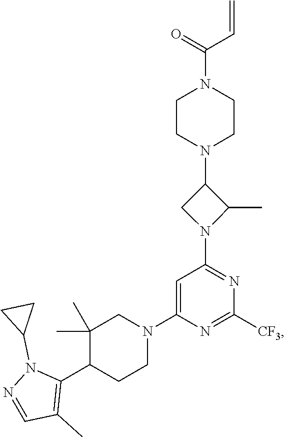 [SMILES: C=CC(=O)N1CCN(C2CN(c3cc(N4CCC(c5c(C)cnn5C5CC5)C(C)(C)C4)nc(C(F)(F)F)n3)C2C)CC1]

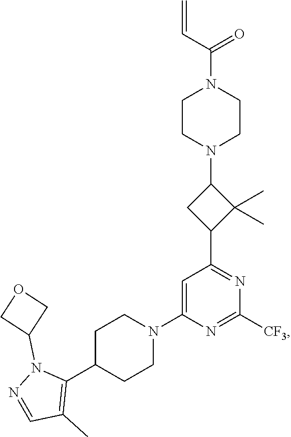 [SMILES: C=CC(=O)N1CCN(C2CC(c3cc(N4CCC(c5c(C)cnn5C5COC5)CC4)nc(C(F)(F)F)n3)C2(C)C)CC1]

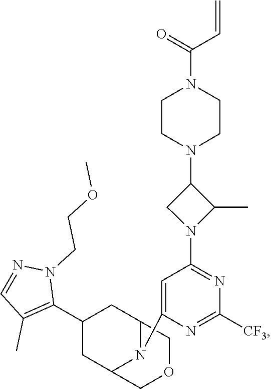 [SMILES: C=CC(=O)N1CCN(C2CN(c3cc(N4C5COCC4CC(c4c(C)cnn4CCOC)C5)nc(C(F)(F)F)n3)C2C)CC1]

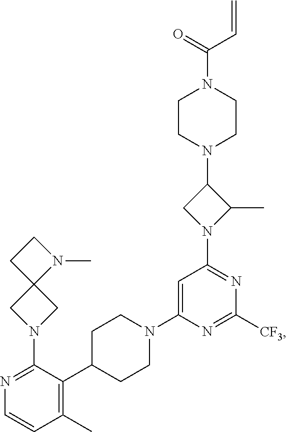 [SMILES: C=CC(=O)N1CCN(C2CN(c3cc(N4CCC(c5c(C)ccnc5N5CC6(CCN6C)C5)CC4)nc(C(F)(F)F)n3)C2C)CC1]

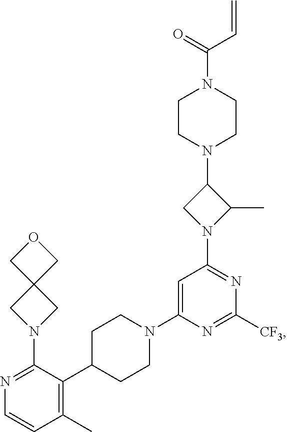 [SMILES: C=CC(=O)N1CCN(C2CN(c3cc(N4CCC(c5c(C)ccnc5N5CC6(COC6)C5)CC4)nc(C(F)(F)F)n3)C2C)CC1]

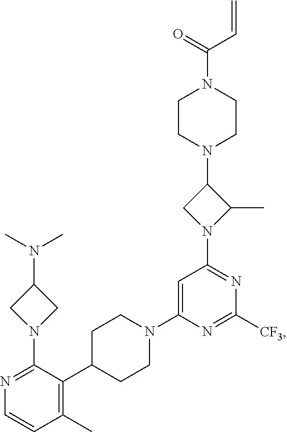 [SMILES: C=CC(=O)N1CCN(C2CN(c3cc(N4CCC(c5c(C)ccnc5N5CC(N(C)C)C5)CC4)nc(C(F)(F)F)n3)C2C)CC1]

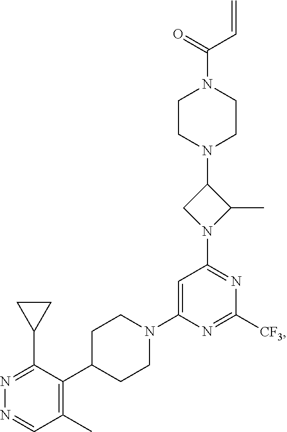 [SMILES: C=CC(=O)N1CCN(C2CN(c3cc(N4CCC(c5c(C)cnnc5C5CC5)CC4)nc(C(F)(F)F)n3)C2C)CC1]

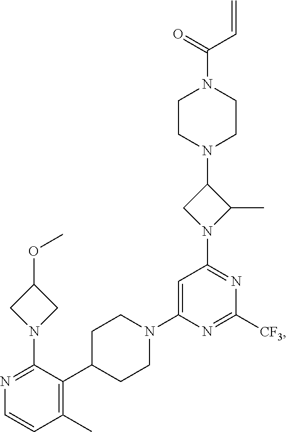 [SMILES: C=CC(=O)N1CCN(C2CN(c3cc(N4CCC(c5c(C)ccnc5N5CC(OC)C5)CC4)nc(C(F)(F)F)n3)C2C)CC1]

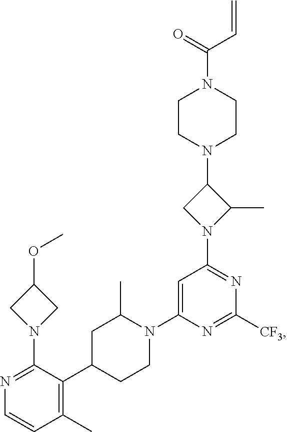 [SMILES: C=CC(=O)N1CCN(C2CN(c3cc(N4CCC(c5c(C)ccnc5N5CC(OC)C5)CC4C)nc(C(F)(F)F)n3)C2C)CC1]

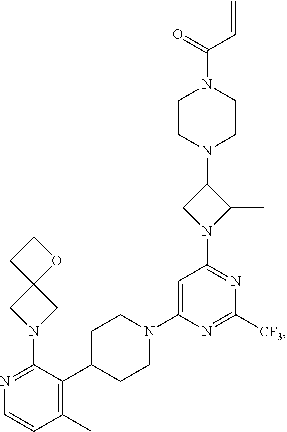 [SMILES: C=CC(=O)N1CCN(C2CN(c3cc(N4CCC(c5c(C)ccnc5N5CC6(CCO6)C5)CC4)nc(C(F)(F)F)n3)C2C)CC1]

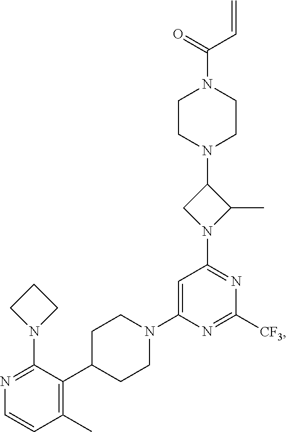 [SMILES: C=CC(=O)N1CCN(C2CN(c3cc(N4CCC(c5c(C)ccnc5N5CCC5)CC4)nc(C(F)(F)F)n3)C2C)CC1]

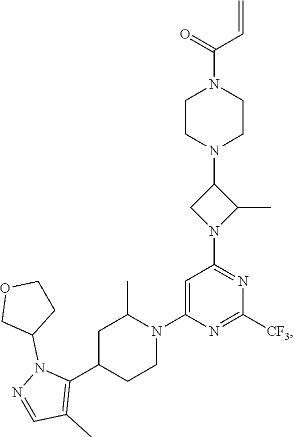 [SMILES: C=CC(=O)N1CCN(C2CN(c3cc(N4CCC(c5c(C)cnn5C5CCOC5)CC4C)nc(C(F)(F)F)n3)C2C)CC1]

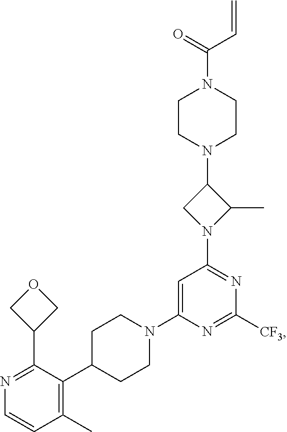 [SMILES: C=CC(=O)N1CCN(C2CN(c3cc(N4CCC(c5c(C)ccnc5C5COC5)CC4)nc(C(F)(F)F)n3)C2C)CC1]

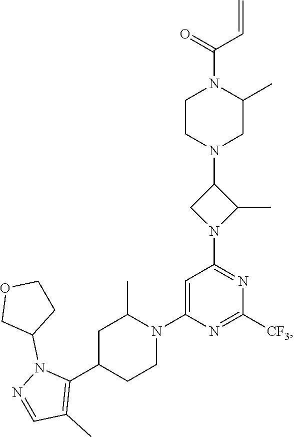 [SMILES: C=CC(=O)N1CCN(C2CN(c3cc(N4CCC(c5c(C)cnn5C5CCOC5)CC4C)nc(C(F)(F)F)n3)C2C)CC1C]

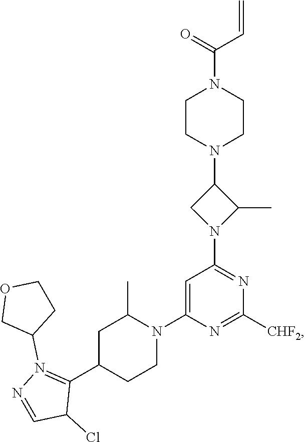

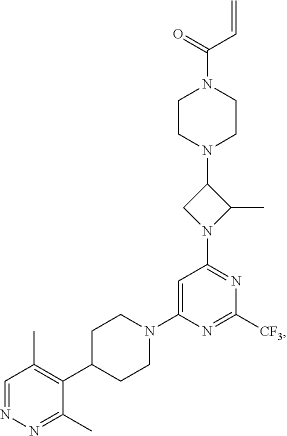 [SMILES: C=CC(=O)N1CCN(C2CN(c3cc(N4CCC(c5c(C)cnnc5C)CC4)nc(C(F)(F)F)n3)C2C)CC1]

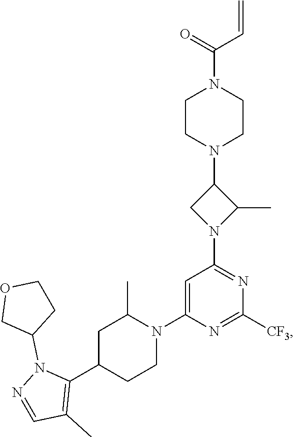 [SMILES: C=CC(=O)N1CCN(C2CN(c3cc(N4CCC(c5c(C)cnn5C5CCOC5)CC4C)nc(C(F)(F)F)n3)C2C)CC1]

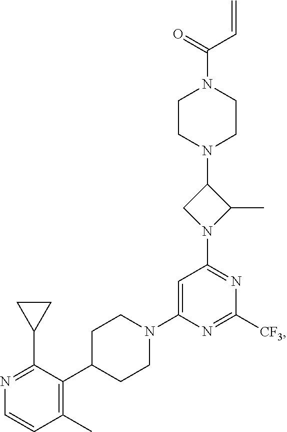 [SMILES: C=CC(=O)N1CCN(C2CN(c3cc(N4CCC(c5c(C)ccnc5C5CC5)CC4)nc(C(F)(F)F)n3)C2C)CC1]

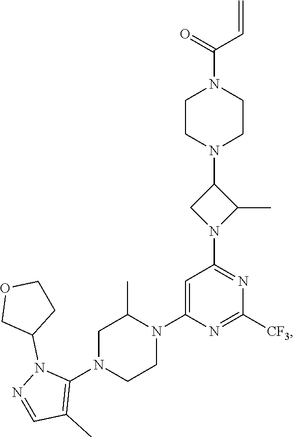 [SMILES: C=CC(=O)N1CCN(C2CN(c3cc(N4CCN(c5c(C)cnn5C5CCOC5)CC4C)nc(C(F)(F)F)n3)C2C)CC1]

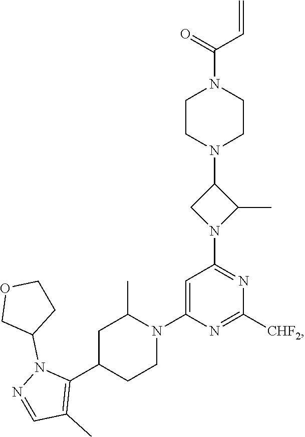 [SMILES: C=CC(=O)N1CCN(C2CN(c3cc(N4CCC(c5c(C)cnn5C5CCOC5)CC4C)nc(C(F)F)n3)C2C)CC1]

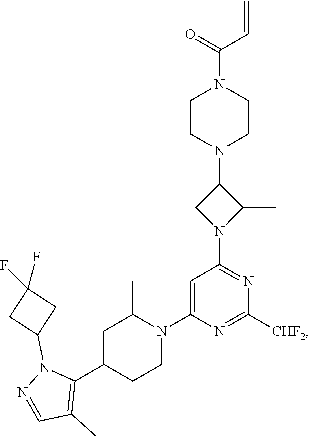 [SMILES: C=CC(=O)N1CCN(C2CN(c3cc(N4CCC(c5c(C)cnn5C5CC(F)(F)C5)CC4C)nc(C(F)F)n3)C2C)CC1]

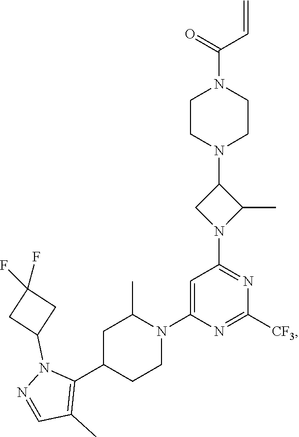 [SMILES: C=CC(=O)N1CCN(C2CN(c3cc(N4CCC(c5c(C)cnn5C5CC(F)(F)C5)CC4C)nc(C(F)(F)F)n3)C2C)CC1]

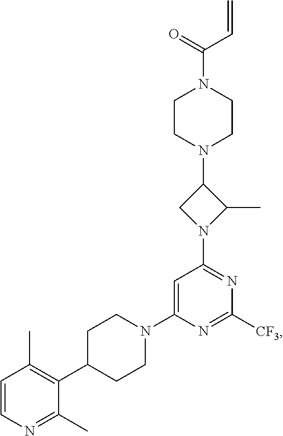 [SMILES: C=CC(=O)N1CCN(C2CN(c3cc(N4CCC(c5c(C)ccnc5C)CC4)nc(C(F)(F)F)n3)C2C)CC1]

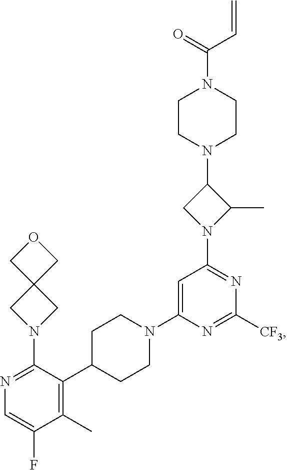 [SMILES: C=CC(=O)N1CCN(C2CN(c3cc(N4CCC(c5c(N6CC7(COC7)C6)ncc(F)c5C)CC4)nc(C(F)(F)F)n3)C2C)CC1]

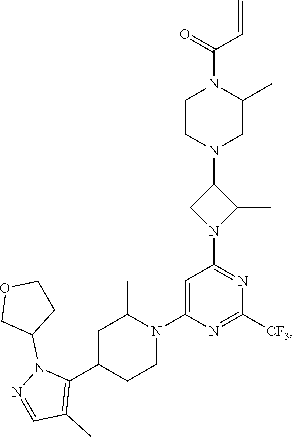 [SMILES: C=CC(=O)N1CCN(C2CN(c3cc(N4CCC(c5c(C)cnn5C5CCOC5)CC4C)nc(C(F)(F)F)n3)C2C)CC1C]

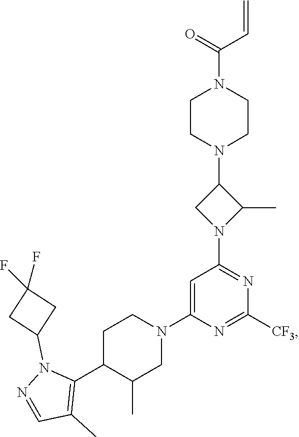 [SMILES: C=CC(=O)N1CCN(C2CN(c3cc(N4CCC(c5c(C)cnn5C5CC(F)(F)C5)C(C)C4)nc(C(F)(F)F)n3)C2C)CC1]

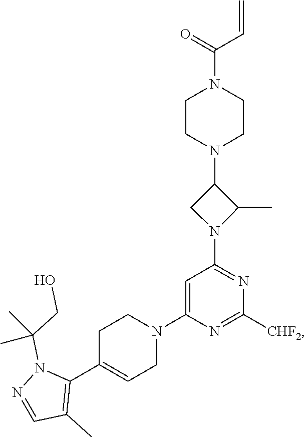 [SMILES: C=CC(=O)N1CCN(C2CN(c3cc(N4CC=C(c5c(C)cnn5C(C)(C)CO)CC4)nc(C(F)F)n3)C2C)CC1]

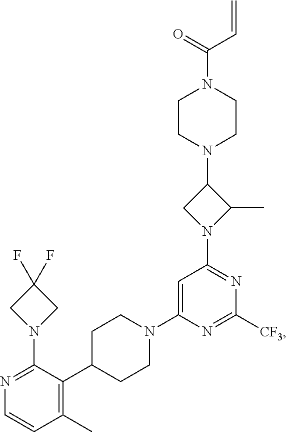 [SMILES: C=CC(=O)N1CCN(C2CN(c3cc(N4CCC(c5c(C)ccnc5N5CC(F)(F)C5)CC4)nc(C(F)(F)F)n3)C2C)CC1]

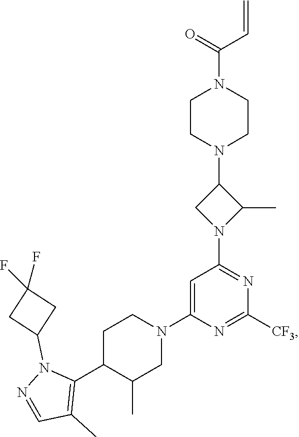 [SMILES: C=CC(=O)N1CCN(C2CN(c3cc(N4CCC(c5c(C)cnn5C5CC(F)(F)C5)C(C)C4)nc(C(F)(F)F)n3)C2C)CC1]

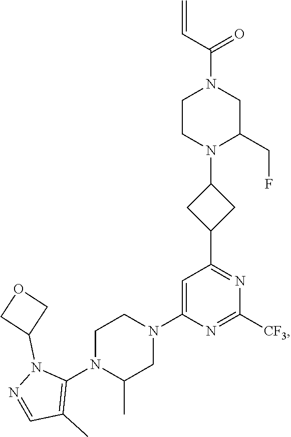 [SMILES: C=CC(=O)N1CCN(C2CC(c3cc(N4CCN(c5c(C)cnn5C5COC5)C(C)C4)nc(C(F)(F)F)n3)C2)C(CF)C1]

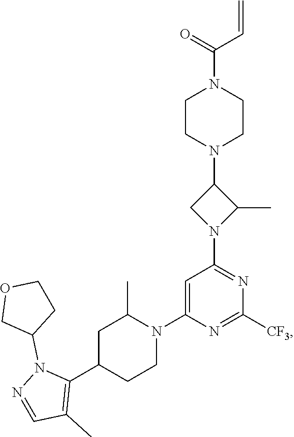 [SMILES: C=CC(=O)N1CCN(C2CN(c3cc(N4CCC(c5c(C)cnn5C5CCOC5)CC4C)nc(C(F)(F)F)n3)C2C)CC1]

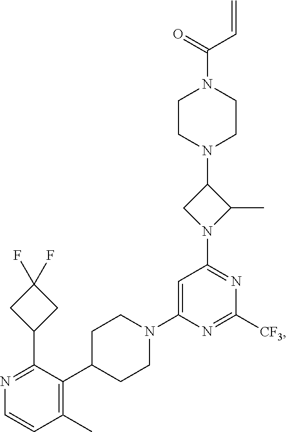 [SMILES: C=CC(=O)N1CCN(C2CN(c3cc(N4CCC(c5c(C)ccnc5C5CC(F)(F)C5)CC4)nc(C(F)(F)F)n3)C2C)CC1]

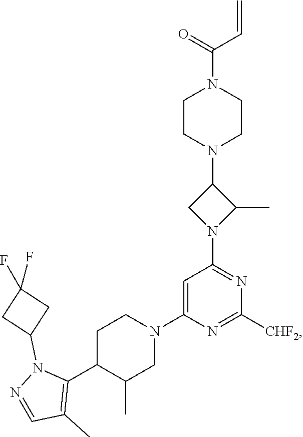 [SMILES: C=CC(=O)N1CCN(C2CN(c3cc(N4CCC(c5c(C)cnn5C5CC(F)(F)C5)C(C)C4)nc(C(F)F)n3)C2C)CC1]

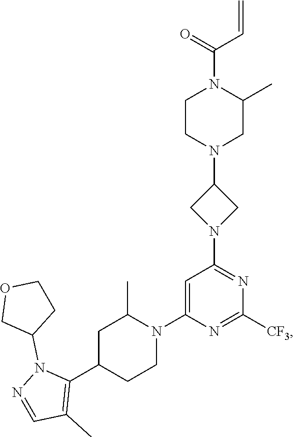 [SMILES: C=CC(=O)N1CCN(C2CN(c3cc(N4CCC(c5c(C)cnn5C5CCOC5)CC4C)nc(C(F)(F)F)n3)C2)CC1C]

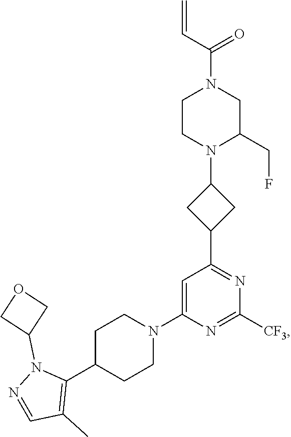 [SMILES: C=CC(=O)N1CCN(C2CC(c3cc(N4CCC(c5c(C)cnn5C5COC5)CC4)nc(C(F)(F)F)n3)C2)C(CF)C1]

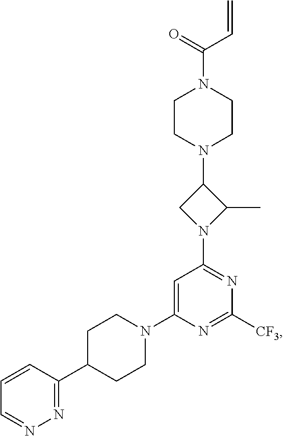 [SMILES: C=CC(=O)N1CCN(C2CN(c3cc(N4CCC(c5cccnn5)CC4)nc(C(F)(F)F)n3)C2C)CC1]

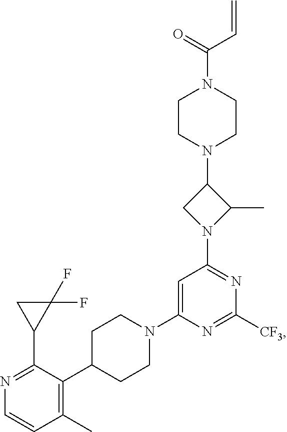 [SMILES: C=CC(=O)N1CCN(C2CN(c3cc(N4CCC(c5c(C)ccnc5C5CC5(F)F)CC4)nc(C(F)(F)F)n3)C2C)CC1]

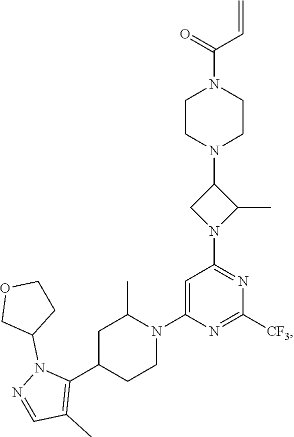 [SMILES: C=CC(=O)N1CCN(C2CN(c3cc(N4CCC(c5c(C)cnn5C5CCOC5)CC4C)nc(C(F)(F)F)n3)C2C)CC1]

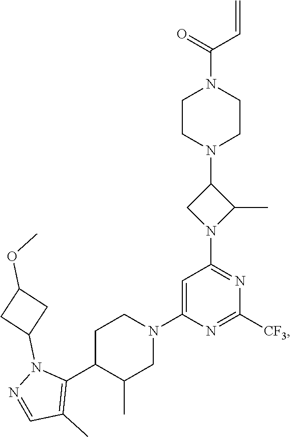 [SMILES: C=CC(=O)N1CCN(C2CN(c3cc(N4CCC(c5c(C)cnn5C5CC(OC)C5)C(C)C4)nc(C(F)(F)F)n3)C2C)CC1]

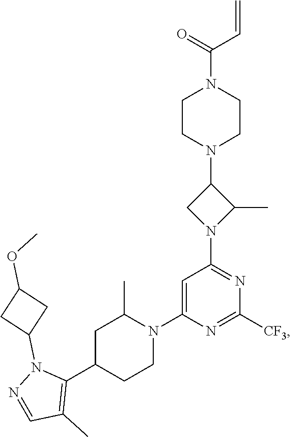 [SMILES: C=CC(=O)N1CCN(C2CN(c3cc(N4CCC(c5c(C)cnn5C5CC(OC)C5)CC4C)nc(C(F)(F)F)n3)C2C)CC1]

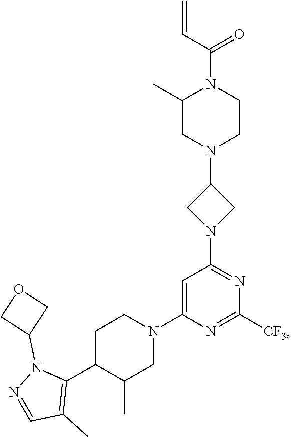 [SMILES: C=CC(=O)N1CCN(C2CN(c3cc(N4CCC(c5c(C)cnn5C5COC5)C(C)C4)nc(C(F)(F)F)n3)C2)CC1C]

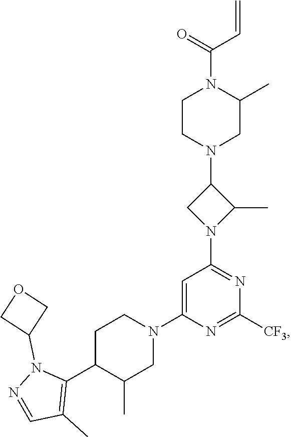 [SMILES: C=CC(=O)N1CCN(C2CN(c3cc(N4CCC(c5c(C)cnn5C5COC5)C(C)C4)nc(C(F)(F)F)n3)C2C)CC1C]

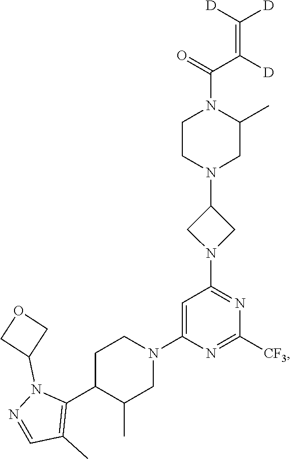 [SMILES: [2H]C([2H])=C([2H])C(=O)N1CCN(C2CN(c3cc(N4CCC(c5c(C)cnn5C5COC5)C(C)C4)nc(C(F)(F)F)n3)C2)CC1C]

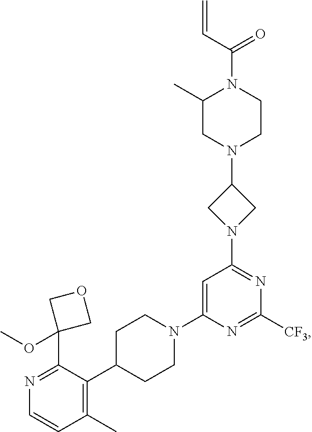 [SMILES: C=CC(=O)N1CCN(C2CN(c3cc(N4CCC(c5c(C)ccnc5C5(OC)COC5)CC4)nc(C(F)(F)F)n3)C2)CC1C]

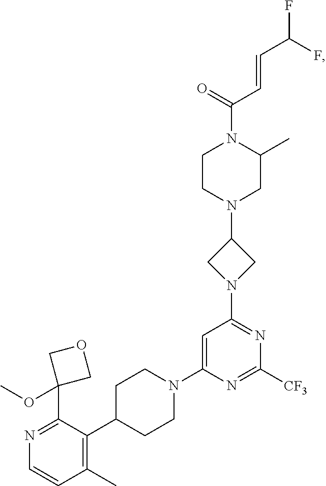 [SMILES: COC1(c2nccc(C)c2C2CCN(c3cc(N4CC(N5CCN(C(=O)/C=C/C(F)F)C(C)C5)C4)nc(C(F)(F)F)n3)CC2)COC1]

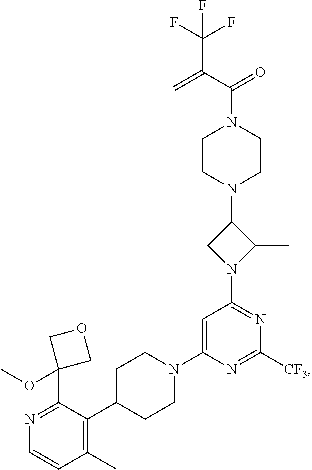 [SMILES: C=C(C(=O)N1CCN(C2CN(c3cc(N4CCC(c5c(C)ccnc5C5(OC)COC5)CC4)nc(C(F)(F)F)n3)C2C)CC1)C(F)(F)F]

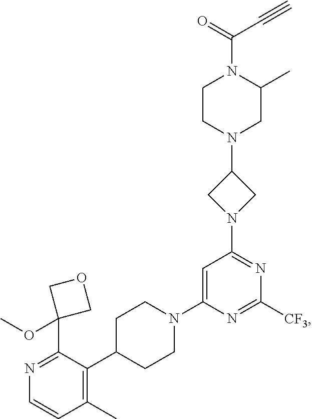 [SMILES: C#CC(=O)N1CCN(C2CN(c3cc(N4CCC(c5c(C)ccnc5C5(OC)COC5)CC4)nc(C(F)(F)F)n3)C2)CC1C]

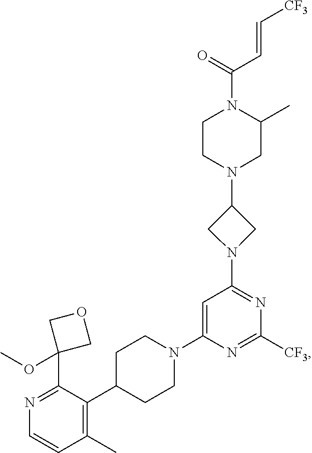 [SMILES: COC1(c2nccc(C)c2C2CCN(c3cc(N4CC(N5CCN(C(=O)/C=C/C(F)(F)F)C(C)C5)C4)nc(C(F)(F)F)n3)CC2)COC1]

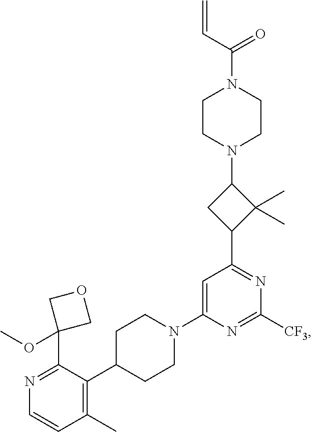 [SMILES: C=CC(=O)N1CCN(C2CC(c3cc(N4CCC(c5c(C)ccnc5C5(OC)COC5)CC4)nc(C(F)(F)F)n3)C2(C)C)CC1]

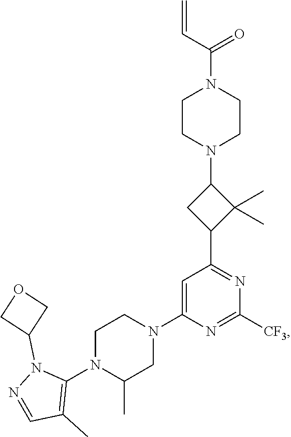 [SMILES: C=CC(=O)N1CCN(C2CC(c3cc(N4CCN(c5c(C)cnn5C5COC5)C(C)C4)nc(C(F)(F)F)n3)C2(C)C)CC1]

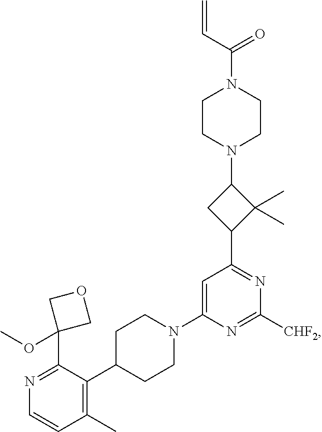 [SMILES: C=CC(=O)N1CCN(C2CC(c3cc(N4CCC(c5c(C)ccnc5C5(OC)COC5)CC4)nc(C(F)F)n3)C2(C)C)CC1]

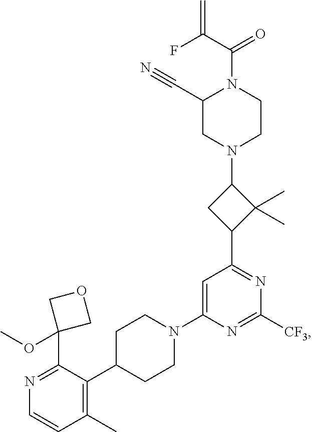 [SMILES: C=C(F)C(=O)N1CCN(C2CC(c3cc(N4CCC(c5c(C)ccnc5C5(OC)COC5)CC4)nc(C(F)(F)F)n3)C2(C)C)CC1C#N]

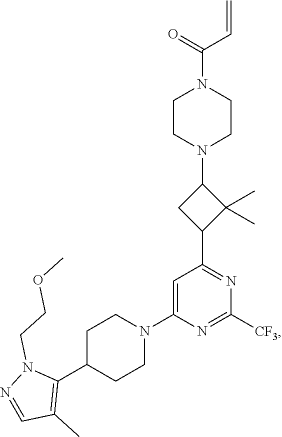 [SMILES: C=CC(=O)N1CCN(C2CC(c3cc(N4CCC(c5c(C)cnn5CCOC)CC4)nc(C(F)(F)F)n3)C2(C)C)CC1]

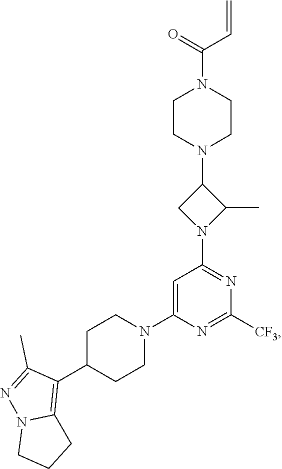 [SMILES: C=CC(=O)N1CCN(C2CN(c3cc(N4CCC(c5c(C)nn6c5CCC6)CC4)nc(C(F)(F)F)n3)C2C)CC1]

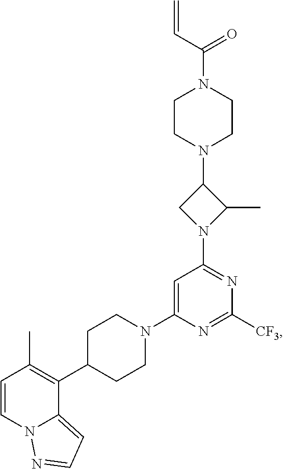 [SMILES: C=CC(=O)N1CCN(C2CN(c3cc(N4CCC(c5c(C)ccn6nccc56)CC4)nc(C(F)(F)F)n3)C2C)CC1]

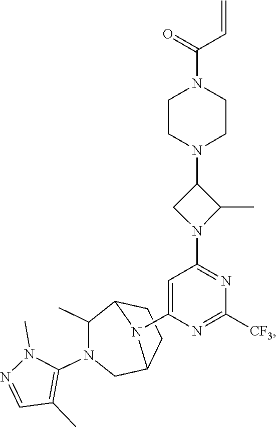 [SMILES: C=CC(=O)N1CCN(C2CN(c3cc(N4C5CCC4C(C)N(c4c(C)cnn4C)C5)nc(C(F)(F)F)n3)C2C)CC1]

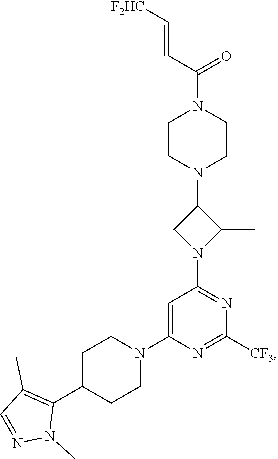 [SMILES: Cc1cnn(C)c1C1CCN(c2cc(N3CC(N4CCN(C(=O)/C=C/C(F)F)CC4)C3C)nc(C(F)(F)F)n2)CC1]

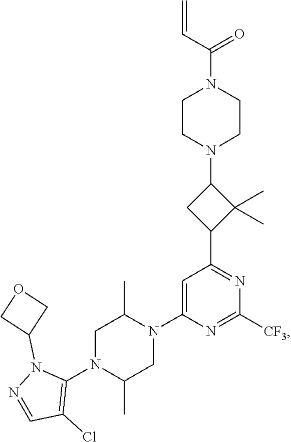 [SMILES: C=CC(=O)N1CCN(C2CC(c3cc(N4CC(C)N(c5c(Cl)cnn5C5COC5)CC4C)nc(C(F)(F)F)n3)C2(C)C)CC1]

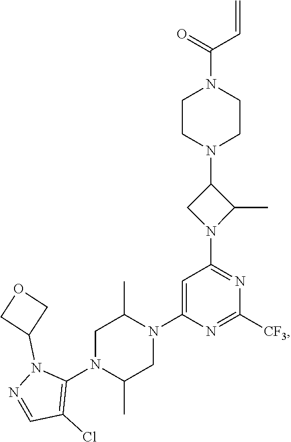 [SMILES: C=CC(=O)N1CCN(C2CN(c3cc(N4CC(C)N(c5c(Cl)cnn5C5COC5)CC4C)nc(C(F)(F)F)n3)C2C)CC1]

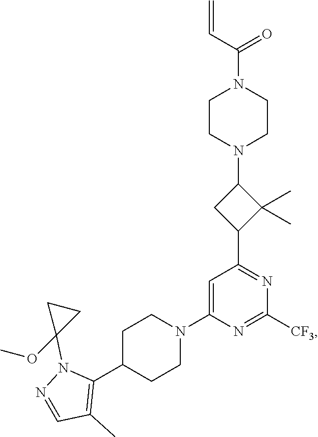 [SMILES: C=CC(=O)N1CCN(C2CC(c3cc(N4CCC(c5c(C)cnn5C5(OC)CC5)CC4)nc(C(F)(F)F)n3)C2(C)C)CC1]

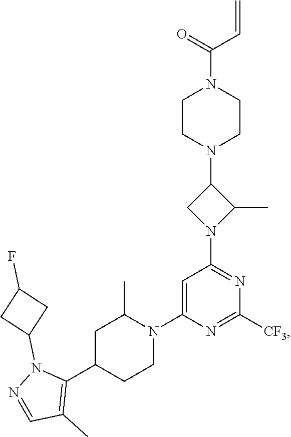 [SMILES: C=CC(=O)N1CCN(C2CN(c3cc(N4CCC(c5c(C)cnn5C5CC(F)C5)CC4C)nc(C(F)(F)F)n3)C2C)CC1]

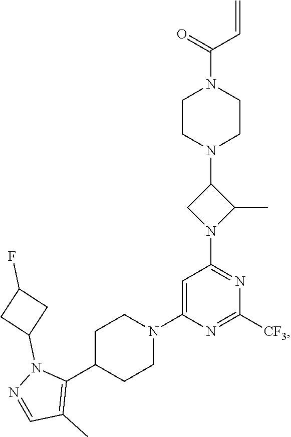 [SMILES: C=CC(=O)N1CCN(C2CN(c3cc(N4CCC(c5c(C)cnn5C5CC(F)C5)CC4)nc(C(F)(F)F)n3)C2C)CC1]

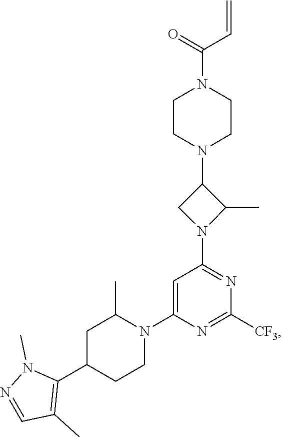 [SMILES: C=CC(=O)N1CCN(C2CN(c3cc(N4CCC(c5c(C)cnn5C)CC4C)nc(C(F)(F)F)n3)C2C)CC1]

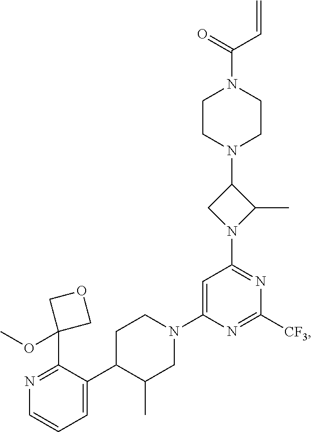 [SMILES: C=CC(=O)N1CCN(C2CN(c3cc(N4CCC(c5cccnc5C5(OC)COC5)C(C)C4)nc(C(F)(F)F)n3)C2C)CC1]

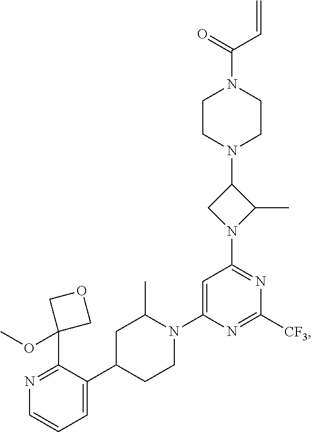 [SMILES: C=CC(=O)N1CCN(C2CN(c3cc(N4CCC(c5cccnc5C5(OC)COC5)CC4C)nc(C(F)(F)F)n3)C2C)CC1]

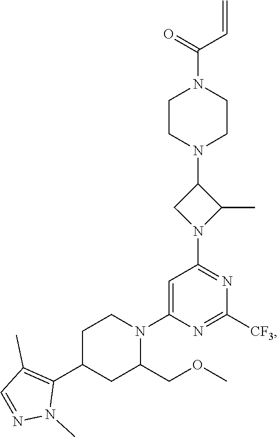 [SMILES: C=CC(=O)N1CCN(C2CN(c3cc(N4CCC(c5c(C)cnn5C)CC4COC)nc(C(F)(F)F)n3)C2C)CC1]

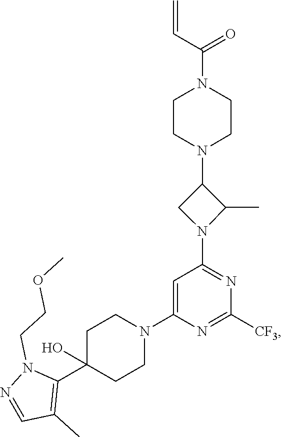 [SMILES: C=CC(=O)N1CCN(C2CN(c3cc(N4CCC(O)(c5c(C)cnn5CCOC)CC4)nc(C(F)(F)F)n3)C2C)CC1]

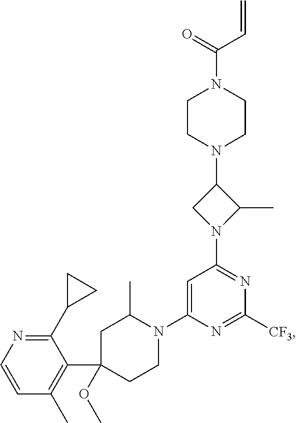 [SMILES: C=CC(=O)N1CCN(C2CN(c3cc(N4CCC(OC)(c5c(C)ccnc5C5CC5)CC4C)nc(C(F)(F)F)n3)C2C)CC1]

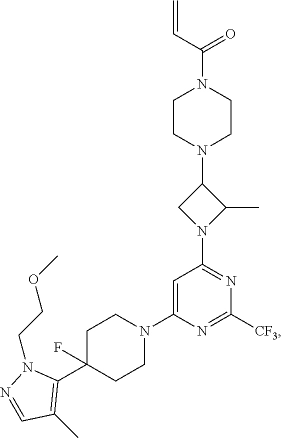 [SMILES: C=CC(=O)N1CCN(C2CN(c3cc(N4CCC(F)(c5c(C)cnn5CCOC)CC4)nc(C(F)(F)F)n3)C2C)CC1]

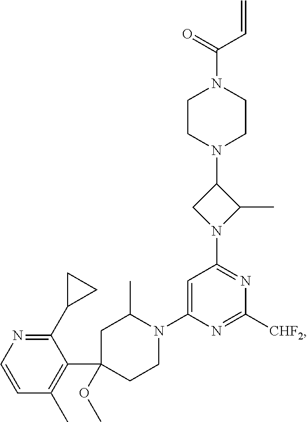 [SMILES: C=CC(=O)N1CCN(C2CN(c3cc(N4CCC(OC)(c5c(C)ccnc5C5CC5)CC4C)nc(C(F)F)n3)C2C)CC1]

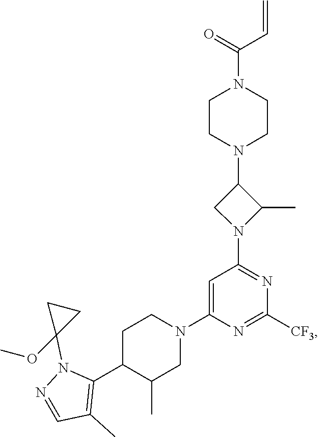 [SMILES: C=CC(=O)N1CCN(C2CN(c3cc(N4CCC(c5c(C)cnn5C5(OC)CC5)C(C)C4)nc(C(F)(F)F)n3)C2C)CC1]

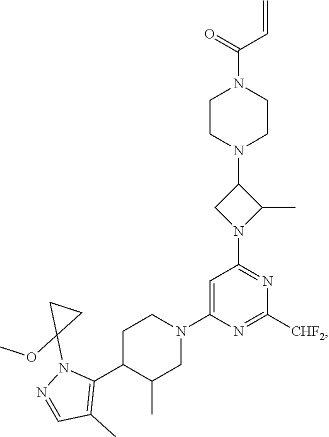 [SMILES: C=CC(=O)N1CCN(C2CN(c3cc(N4CCC(c5c(C)cnn5C5(OC)CC5)C(C)C4)nc(C(F)F)n3)C2C)CC1]

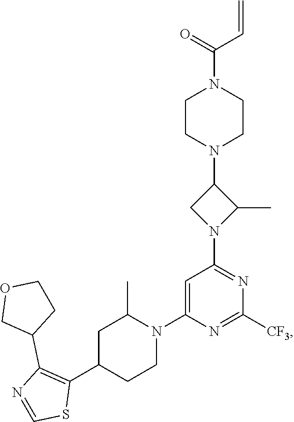 [SMILES: C=CC(=O)N1CCN(C2CN(c3cc(N4CCC(c5scnc5C5CCOC5)CC4C)nc(C(F)(F)F)n3)C2C)CC1]

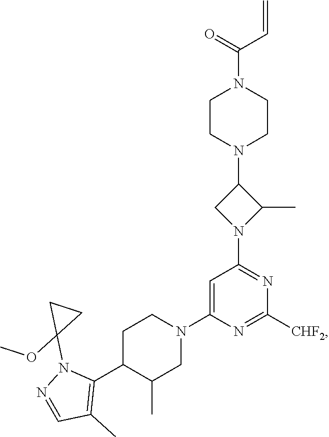 [SMILES: C=CC(=O)N1CCN(C2CN(c3cc(N4CCC(c5c(C)cnn5C5(OC)CC5)C(C)C4)nc(C(F)F)n3)C2C)CC1]

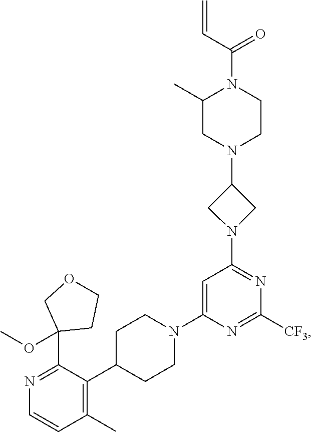

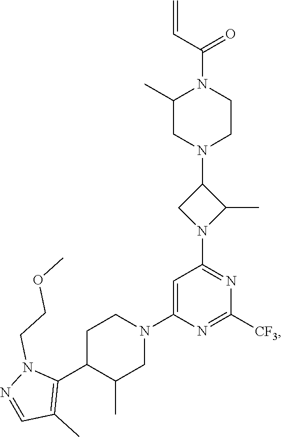 [SMILES: C=CC(=O)N1CCN(C2CN(c3cc(N4CCC(c5c(C)cnn5CCOC)C(C)C4)nc(C(F)(F)F)n3)C2C)CC1C]

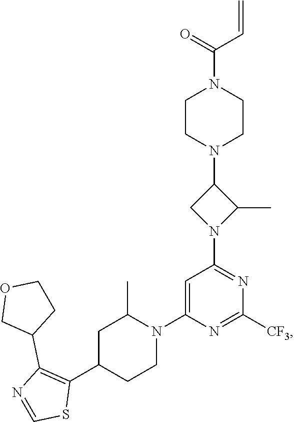 [SMILES: C=CC(=O)N1CCN(C2CN(c3cc(N4CCC(c5scnc5C5CCOC5)CC4C)nc(C(F)(F)F)n3)C2C)CC1]

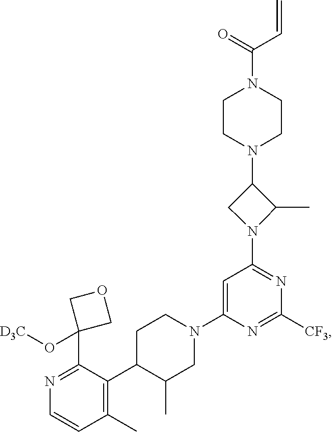 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2C)COC1]

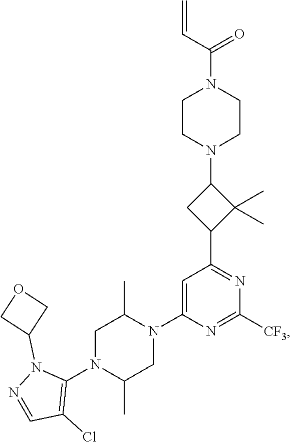 [SMILES: C=CC(=O)N1CCN(C2CC(c3cc(N4CC(C)N(c5c(Cl)cnn5C5COC5)CC4C)nc(C(F)(F)F)n3)C2(C)C)CC1]

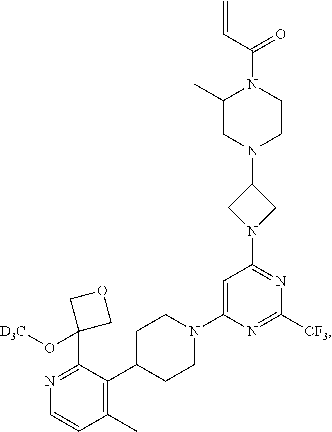 [SMILES: [2H]C([2H])([2H])OC1(c2nccc(C)c2C2CCN(c3cc(N4CC(N5CCN(C(=O)C=C)C(C)C5)C4)nc(C(F)(F)F)n3)CC2)COC1]

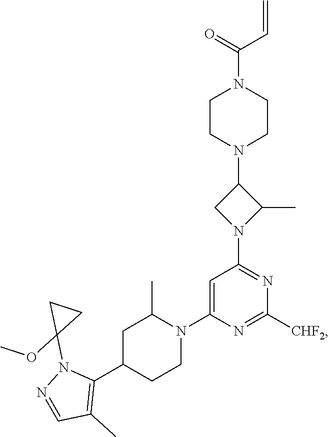 [SMILES: C=CC(=O)N1CCN(C2CN(c3cc(N4CCC(c5c(C)cnn5C5(OC)CC5)CC4C)nc(C(F)F)n3)C2C)CC1]

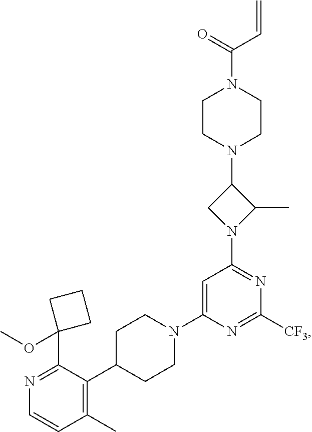 [SMILES: C=CC(=O)N1CCN(C2CN(c3cc(N4CCC(c5c(C)ccnc5C5(OC)CCC5)CC4)nc(C(F)(F)F)n3)C2C)CC1]

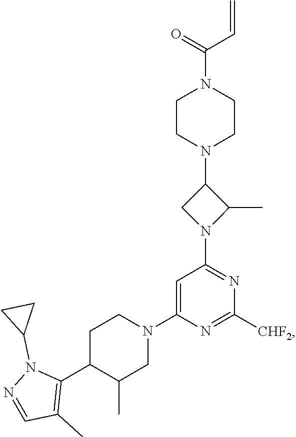 [SMILES: C=CC(=O)N1CCN(C2CN(c3cc(N4CCC(c5c(C)cnn5C5CC5)C(C)C4)nc(C(F)F)n3)C2C)CC1]

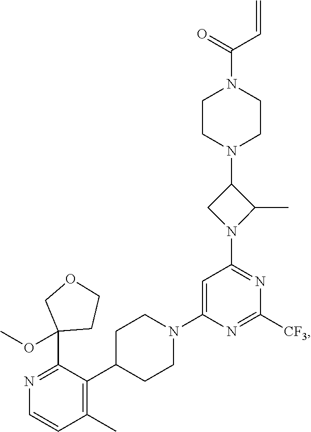 [SMILES: C=CC(=O)N1CCN(C2CN(c3cc(N4CCC(c5c(C)ccnc5C5(OC)CCOC5)CC4)nc(C(F)(F)F)n3)C2C)CC1]

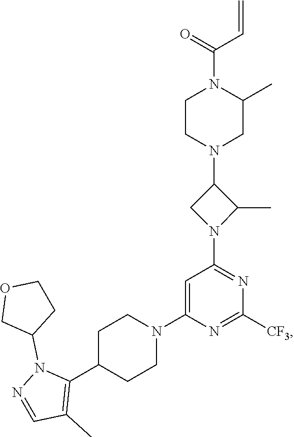 [SMILES: C=CC(=O)N1CCN(C2CN(c3cc(N4CCC(c5c(C)cnn5C5CCOC5)CC4)nc(C(F)(F)F)n3)C2C)CC1C]

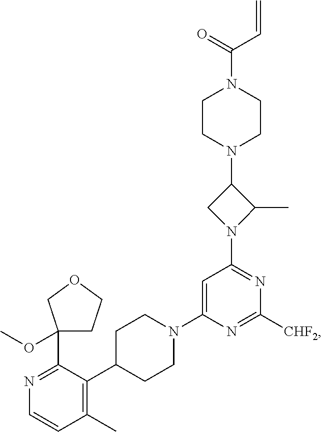 [SMILES: C=CC(=O)N1CCN(C2CN(c3cc(N4CCC(c5c(C)ccnc5C5(OC)CCOC5)CC4)nc(C(F)F)n3)C2C)CC1]

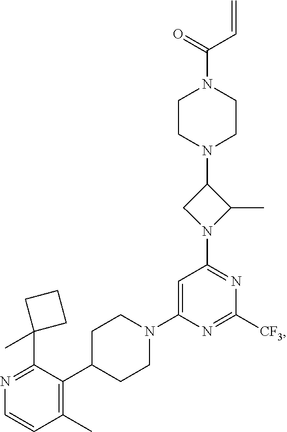 [SMILES: C=CC(=O)N1CCN(C2CN(c3cc(N4CCC(c5c(C)ccnc5C5(C)CCC5)CC4)nc(C(F)(F)F)n3)C2C)CC1]

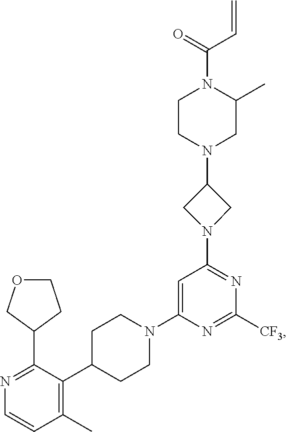 [SMILES: C=CC(=O)N1CCN(C2CN(c3cc(N4CCC(c5c(C)ccnc5C5CCOC5)CC4)nc(C(F)(F)F)n3)C2)CC1C]

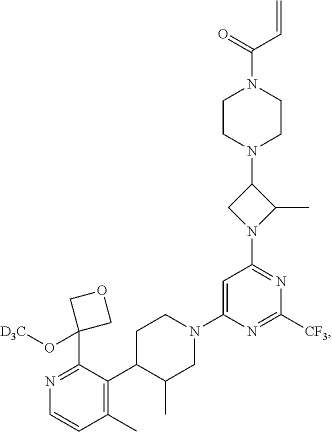 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2C)COC1]

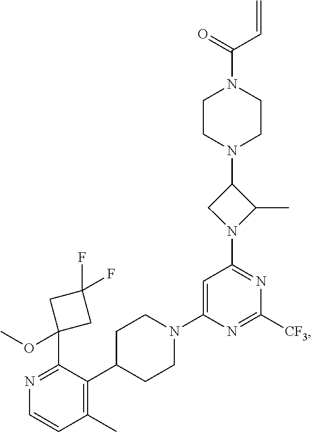 [SMILES: C=CC(=O)N1CCN(C2CN(c3cc(N4CCC(c5c(C)ccnc5C5(OC)CC(F)(F)C5)CC4)nc(C(F)(F)F)n3)C2C)CC1]

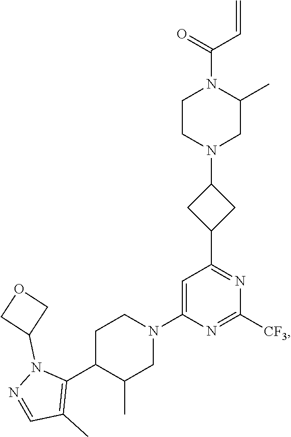 [SMILES: C=CC(=O)N1CCN(C2CC(c3cc(N4CCC(c5c(C)cnn5C5COC5)C(C)C4)nc(C(F)(F)F)n3)C2)CC1C]

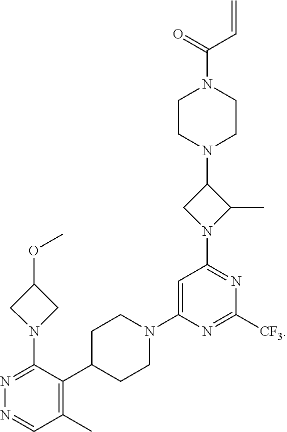 [SMILES: C=CC(=O)N1CCN(C2CN(c3cc(N4CCC(c5c(C)cnnc5N5CC(OC)C5)CC4)nc(C(F)(F)F)n3)C2C)CC1]

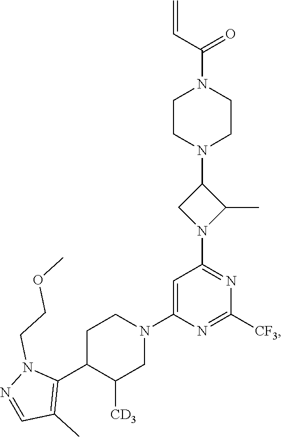 [SMILES: [2H]C([2H])([2H])C1CN(c2cc(N3CC(N4CCN(C(=O)C=C)CC4)C3C)nc(C(F)(F)F)n2)CCC1c1c(C)cnn1CCOC]

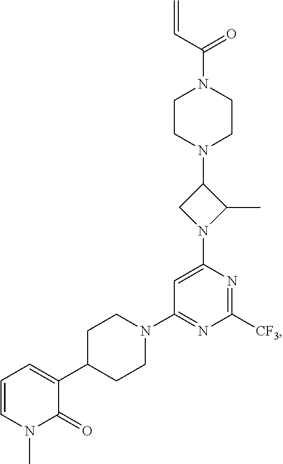 [SMILES: C=CC(=O)N1CCN(C2CN(c3cc(N4CCC(c5cccn(C)c5=O)CC4)nc(C(F)(F)F)n3)C2C)CC1]

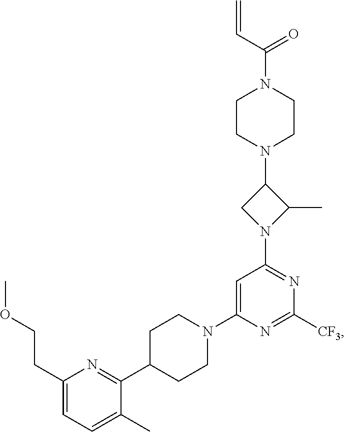 [SMILES: C=CC(=O)N1CCN(C2CN(c3cc(N4CCC(c5nc(CCOC)ccc5C)CC4)nc(C(F)(F)F)n3)C2C)CC1]

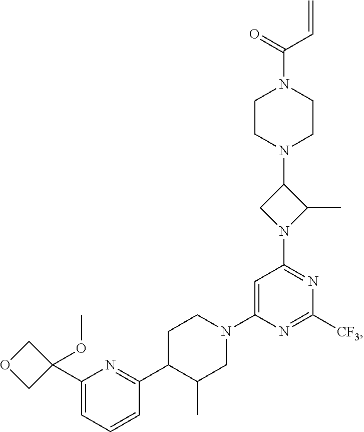 [SMILES: C=CC(=O)N1CCN(C2CN(c3cc(N4CCC(c5cccc(C6(OC)COC6)n5)C(C)C4)nc(C(F)(F)F)n3)C2C)CC1]

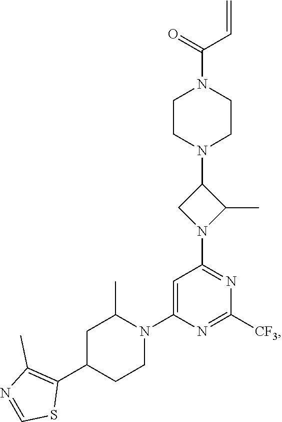 [SMILES: C=CC(=O)N1CCN(C2CN(c3cc(N4CCC(c5scnc5C)CC4C)nc(C(F)(F)F)n3)C2C)CC1]

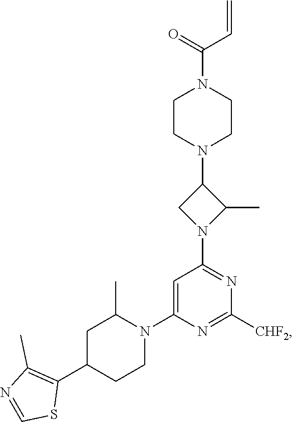 [SMILES: C=CC(=O)N1CCN(C2CN(c3cc(N4CCC(c5scnc5C)CC4C)nc(C(F)F)n3)C2C)CC1]

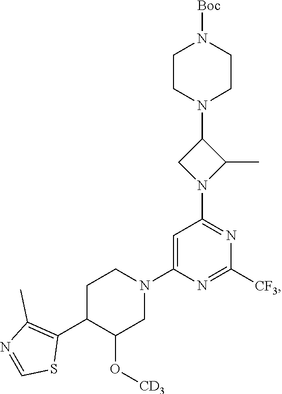 [SMILES: [2H]C([2H])([2H])OC1CN(c2cc(N3CC(N4CCN(C(=O)OC(C)(C)C)CC4)C3C)nc(C(F)(F)F)n2)CCC1c1scnc1C]

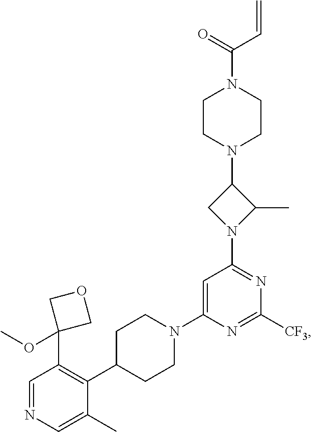 [SMILES: C=CC(=O)N1CCN(C2CN(c3cc(N4CCC(c5c(C)cncc5C5(OC)COC5)CC4)nc(C(F)(F)F)n3)C2C)CC1]

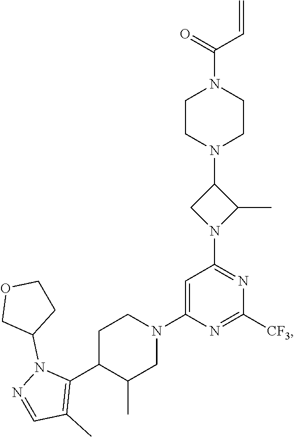 [SMILES: C=CC(=O)N1CCN(C2CN(c3cc(N4CCC(c5c(C)cnn5C5CCOC5)C(C)C4)nc(C(F)(F)F)n3)C2C)CC1]

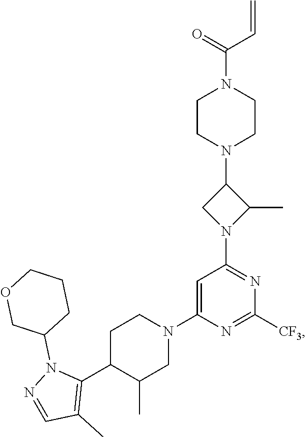 [SMILES: C=CC(=O)N1CCN(C2CN(c3cc(N4CCC(c5c(C)cnn5C5CCCOC5)C(C)C4)nc(C(F)(F)F)n3)C2C)CC1]

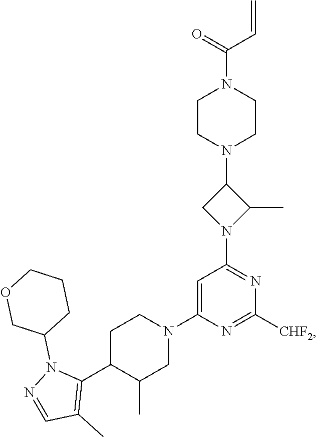 [SMILES: C=CC(=O)N1CCN(C2CN(c3cc(N4CCC(c5c(C)cnn5C5CCCOC5)C(C)C4)nc(C(F)F)n3)C2C)CC1]

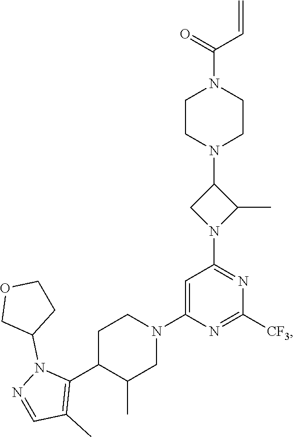 [SMILES: C=CC(=O)N1CCN(C2CN(c3cc(N4CCC(c5c(C)cnn5C5CCOC5)C(C)C4)nc(C(F)(F)F)n3)C2C)CC1]

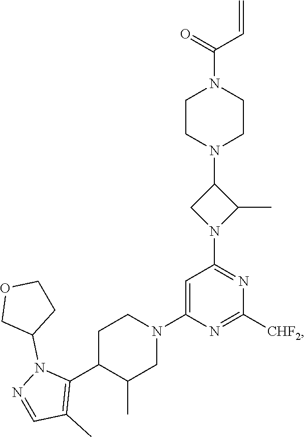 [SMILES: C=CC(=O)N1CCN(C2CN(c3cc(N4CCC(c5c(C)cnn5C5CCOC5)C(C)C4)nc(C(F)F)n3)C2C)CC1]

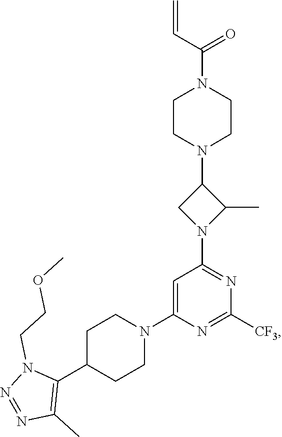 [SMILES: C=CC(=O)N1CCN(C2CN(c3cc(N4CCC(c5c(C)nnn5CCOC)CC4)nc(C(F)(F)F)n3)C2C)CC1]

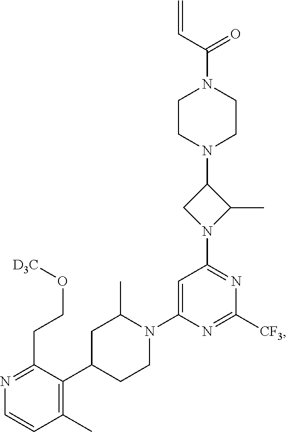 [SMILES: [2H]C([2H])([2H])OCCc1nccc(C)c1C1CCN(c2cc(N3CC(N4CCN(C(=O)C=C)CC4)C3C)nc(C(F)(F)F)n2)C(C)C1]

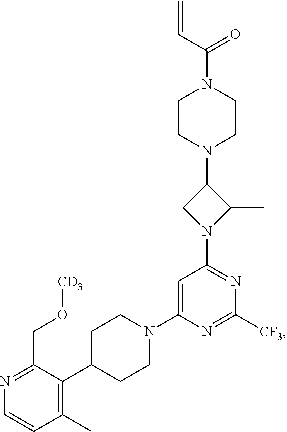 [SMILES: [2H]C([2H])([2H])OCc1nccc(C)c1C1CCN(c2cc(N3CC(N4CCN(C(=O)C=C)CC4)C3C)nc(C(F)(F)F)n2)CC1]

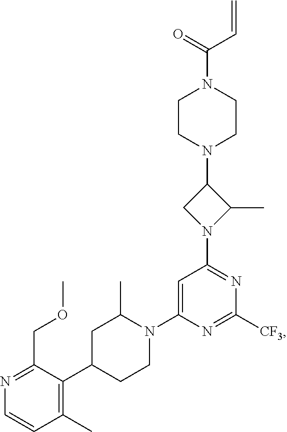 [SMILES: C=CC(=O)N1CCN(C2CN(c3cc(N4CCC(c5c(C)ccnc5COC)CC4C)nc(C(F)(F)F)n3)C2C)CC1]

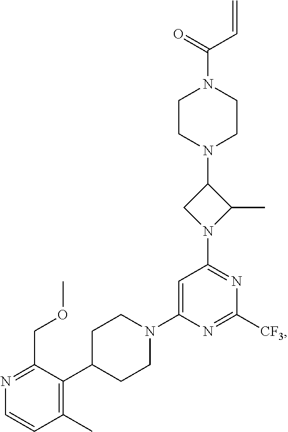 [SMILES: C=CC(=O)N1CCN(C2CN(c3cc(N4CCC(c5c(C)ccnc5COC)CC4)nc(C(F)(F)F)n3)C2C)CC1]

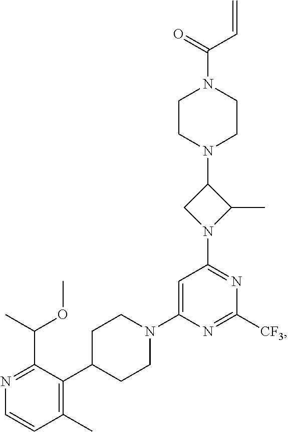 [SMILES: C=CC(=O)N1CCN(C2CN(c3cc(N4CCC(c5c(C)ccnc5C(C)OC)CC4)nc(C(F)(F)F)n3)C2C)CC1]

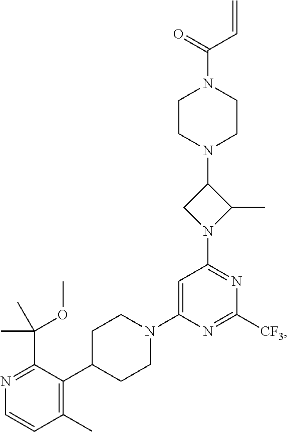 [SMILES: C=CC(=O)N1CCN(C2CN(c3cc(N4CCC(c5c(C)ccnc5C(C)(C)OC)CC4)nc(C(F)(F)F)n3)C2C)CC1]

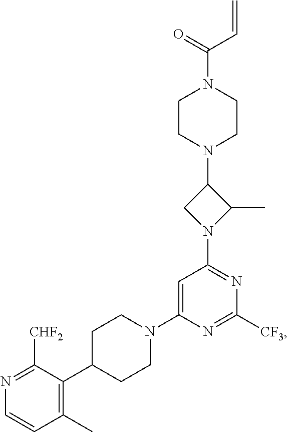 [SMILES: C=CC(=O)N1CCN(C2CN(c3cc(N4CCC(c5c(C)ccnc5C(F)F)CC4)nc(C(F)(F)F)n3)C2C)CC1]

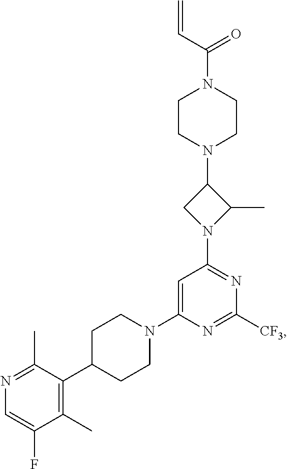 [SMILES: C=CC(=O)N1CCN(C2CN(c3cc(N4CCC(c5c(C)ncc(F)c5C)CC4)nc(C(F)(F)F)n3)C2C)CC1]

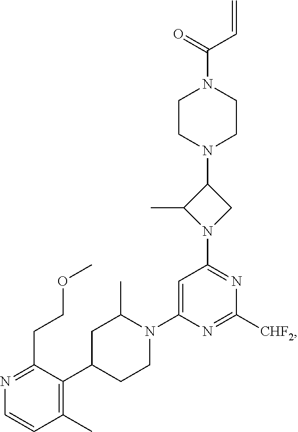 [SMILES: C=CC(=O)N1CCN(C2CN(c3cc(N4CCC(c5c(C)ccnc5CCOC)CC4C)nc(C(F)F)n3)C2C)CC1]

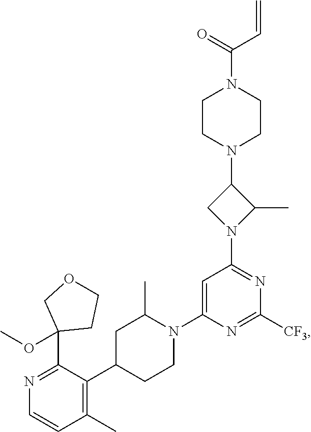 [SMILES: C=CC(=O)N1CCN(C2CN(c3cc(N4CCC(c5c(C)ccnc5C5(OC)CCOC5)CC4C)nc(C(F)(F)F)n3)C2C)CC1]

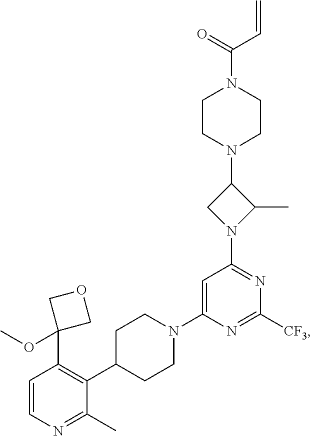 [SMILES: C=CC(=O)N1CCN(C2CN(c3cc(N4CCC(c5c(C6(OC)COC6)ccnc5C)CC4)nc(C(F)(F)F)n3)C2C)CC1]

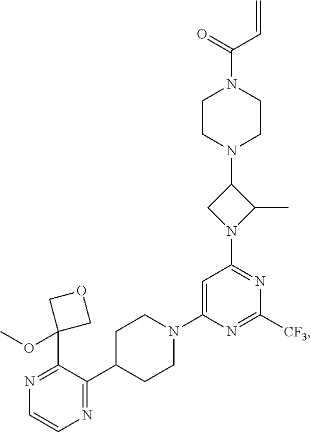 [SMILES: C=CC(=O)N1CCN(C2CN(c3cc(N4CCC(c5nccnc5C5(OC)COC5)CC4)nc(C(F)(F)F)n3)C2C)CC1]

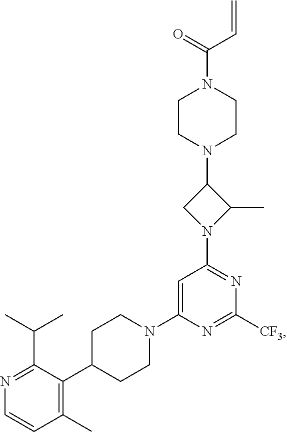 [SMILES: C=CC(=O)N1CCN(C2CN(c3cc(N4CCC(c5c(C)ccnc5C(C)C)CC4)nc(C(F)(F)F)n3)C2C)CC1]

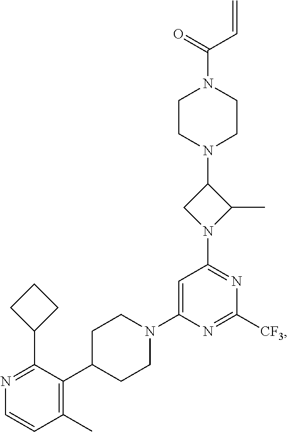 [SMILES: C=CC(=O)N1CCN(C2CN(c3cc(N4CCC(c5c(C)ccnc5C5CCC5)CC4)nc(C(F)(F)F)n3)C2C)CC1]

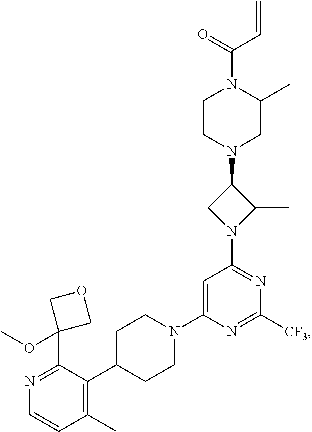 [SMILES: C=CC(=O)N1CCN([C@@H]2CN(c3cc(N4CCC(c5c(C)ccnc5C5(OC)COC5)CC4)nc(C(F)(F)F)n3)C2C)CC1C]

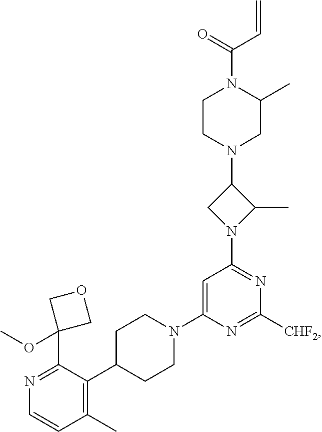 [SMILES: C=CC(=O)N1CCN(C2CN(c3cc(N4CCC(c5c(C)ccnc5C5(OC)COC5)CC4)nc(C(F)F)n3)C2C)CC1C]

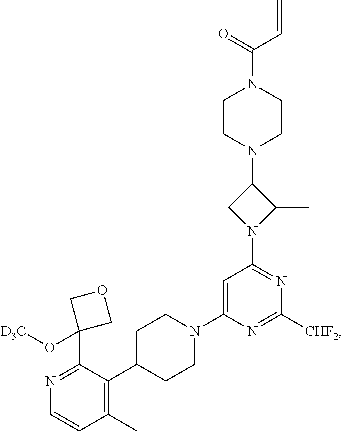 [SMILES: [2H]C([2H])([2H])OC1(c2nccc(C)c2C2CCN(c3cc(N4CC(N5CCN(C(=O)C=C)CC5)C4C)nc(C(F)F)n3)CC2)COC1]

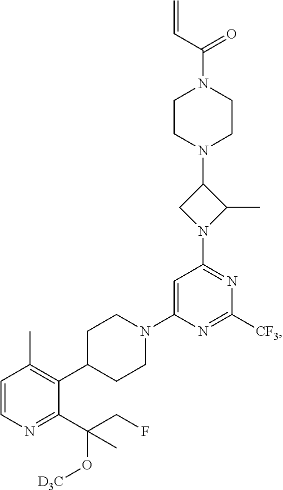 [SMILES: [2H]C([2H])([2H])OC(C)(CF)c1nccc(C)c1C1CCN(c2cc(N3CC(N4CCN(C(=O)C=C)CC4)C3C)nc(C(F)(F)F)n2)CC1]

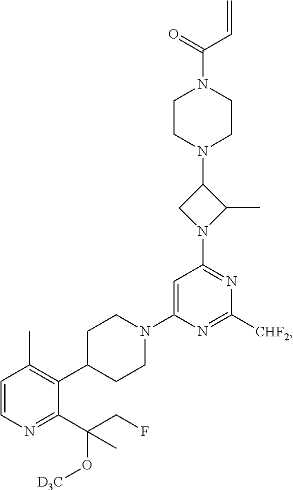 [SMILES: [2H]C([2H])([2H])OC(C)(CF)c1nccc(C)c1C1CCN(c2cc(N3CC(N4CCN(C(=O)C=C)CC4)C3C)nc(C(F)F)n2)CC1]

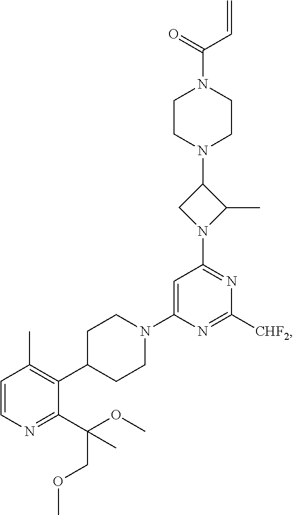 [SMILES: C=CC(=O)N1CCN(C2CN(c3cc(N4CCC(c5c(C)ccnc5C(C)(COC)OC)CC4)nc(C(F)F)n3)C2C)CC1]

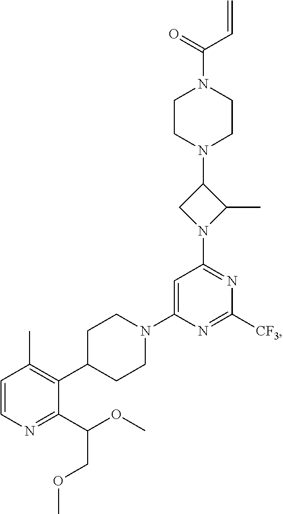 [SMILES: C=CC(=O)N1CCN(C2CN(c3cc(N4CCC(c5c(C)ccnc5C(COC)OC)CC4)nc(C(F)(F)F)n3)C2C)CC1]

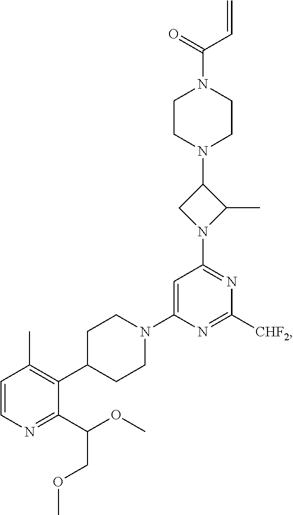 [SMILES: C=CC(=O)N1CCN(C2CN(c3cc(N4CCC(c5c(C)ccnc5C(COC)OC)CC4)nc(C(F)F)n3)C2C)CC1]

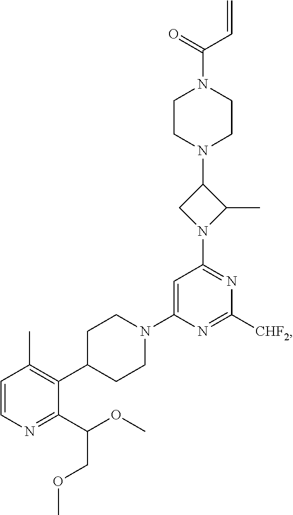 [SMILES: C=CC(=O)N1CCN(C2CN(c3cc(N4CCC(c5c(C)ccnc5C(COC)OC)CC4)nc(C(F)F)n3)C2C)CC1]

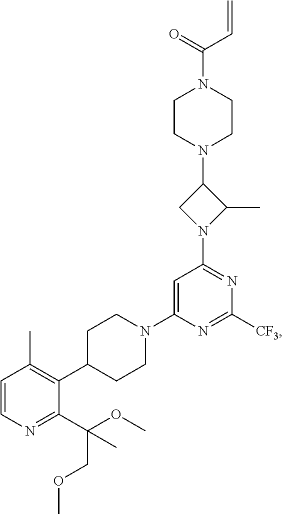 [SMILES: C=CC(=O)N1CCN(C2CN(c3cc(N4CCC(c5c(C)ccnc5C(C)(COC)OC)CC4)nc(C(F)(F)F)n3)C2C)CC1]

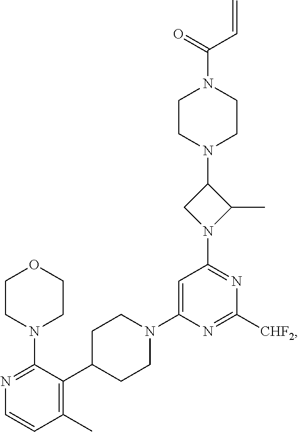 [SMILES: C=CC(=O)N1CCN(C2CN(c3cc(N4CCC(c5c(C)ccnc5N5CCOCC5)CC4)nc(C(F)F)n3)C2C)CC1]

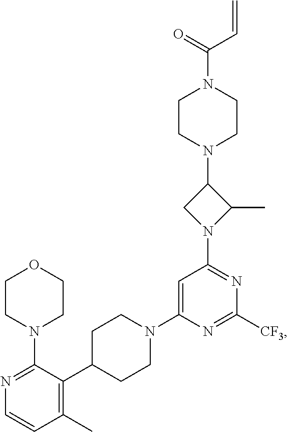 [SMILES: C=CC(=O)N1CCN(C2CN(c3cc(N4CCC(c5c(C)ccnc5N5CCOCC5)CC4)nc(C(F)(F)F)n3)C2C)CC1]

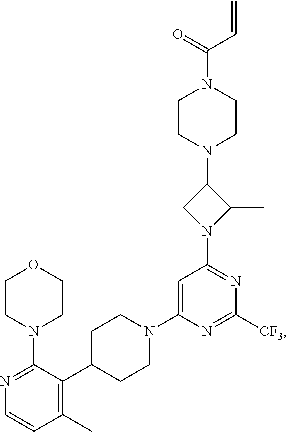 [SMILES: C=CC(=O)N1CCN(C2CN(c3cc(N4CCC(c5c(C)ccnc5N5CCOCC5)CC4)nc(C(F)(F)F)n3)C2C)CC1]

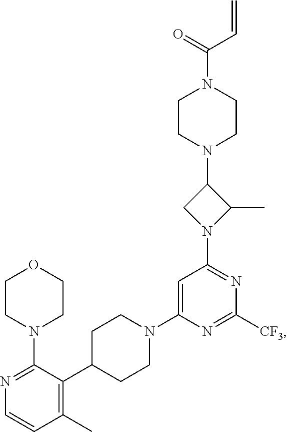 [SMILES: C=CC(=O)N1CCN(C2CN(c3cc(N4CCC(c5c(C)ccnc5N5CCOCC5)CC4)nc(C(F)(F)F)n3)C2C)CC1]

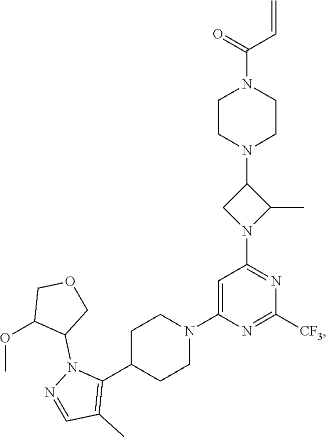 [SMILES: C=CC(=O)N1CCN(C2CN(c3cc(N4CCC(c5c(C)cnn5C5COCC5OC)CC4)nc(C(F)(F)F)n3)C2C)CC1]

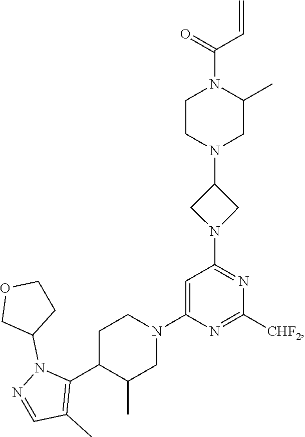 [SMILES: C=CC(=O)N1CCN(C2CN(c3cc(N4CCC(c5c(C)cnn5C5CCOC5)C(C)C4)nc(C(F)F)n3)C2)CC1C]

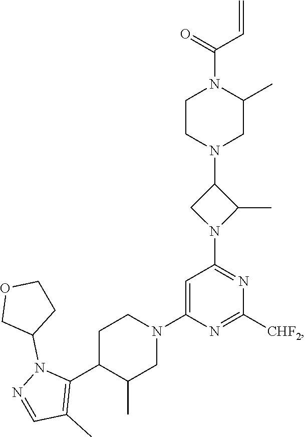 [SMILES: C=CC(=O)N1CCN(C2CN(c3cc(N4CCC(c5c(C)cnn5C5CCOC5)C(C)C4)nc(C(F)F)n3)C2C)CC1C]

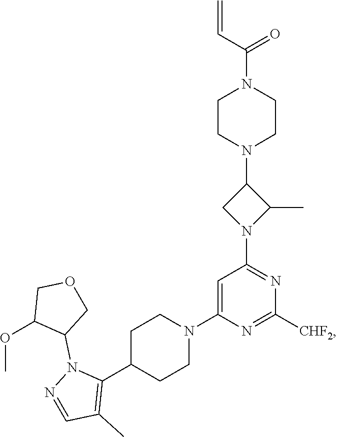 [SMILES: C=CC(=O)N1CCN(C2CN(c3cc(N4CCC(c5c(C)cnn5C5COCC5OC)CC4)nc(C(F)F)n3)C2C)CC1]

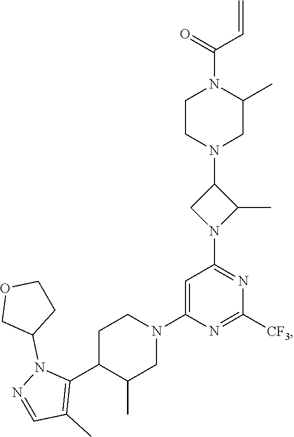 [SMILES: C=CC(=O)N1CCN(C2CN(c3cc(N4CCC(c5c(C)cnn5C5CCOC5)C(C)C4)nc(C(F)(F)F)n3)C2C)CC1C]

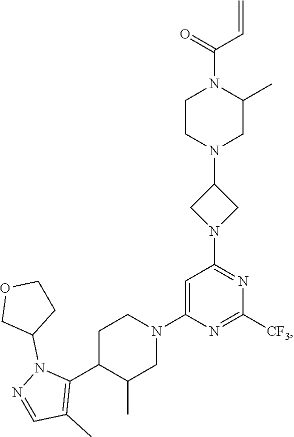 [SMILES: C=CC(=O)N1CCN(C2CN(c3cc(N4CCC(c5c(C)cnn5C5CCOC5)C(C)C4)nc(C(F)(F)F)n3)C2)CC1C]

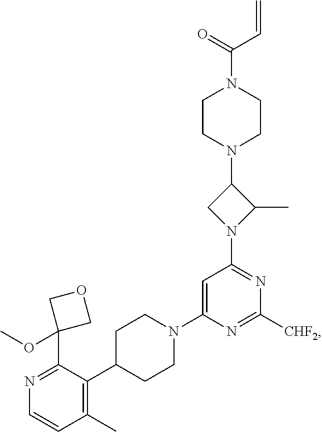 [SMILES: C=CC(=O)N1CCN(C2CN(c3cc(N4CCC(c5c(C)ccnc5C5(OC)COC5)CC4)nc(C(F)F)n3)C2C)CC1]

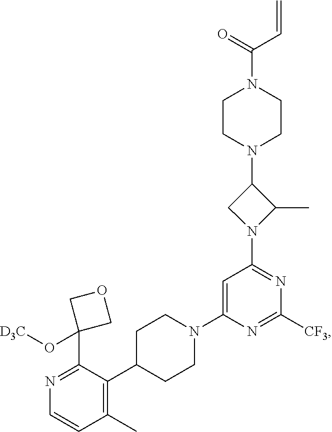 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2)COC1]

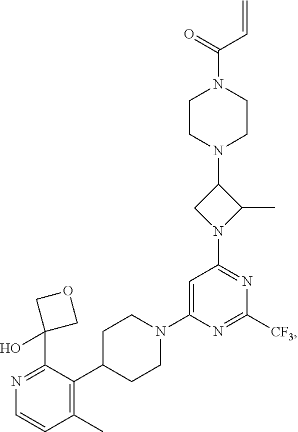 [SMILES: C=CC(=O)N1CCN(C2CN(c3cc(N4CCC(c5c(C)ccnc5C5(O)COC5)CC4)nc(C(F)(F)F)n3)C2C)CC1]

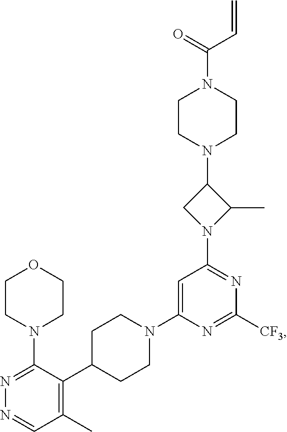 [SMILES: C=CC(=O)N1CCN(C2CN(c3cc(N4CCC(c5c(C)cnnc5N5CCOCC5)CC4)nc(C(F)(F)F)n3)C2C)CC1]

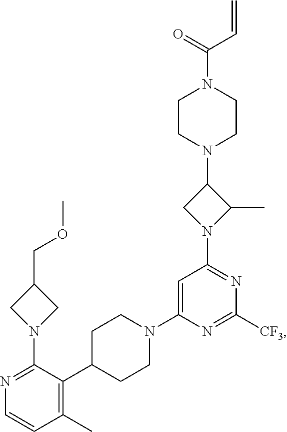 [SMILES: C=CC(=O)N1CCN(C2CN(c3cc(N4CCC(c5c(C)ccnc5N5CC(COC)C5)CC4)nc(C(F)(F)F)n3)C2C)CC1]

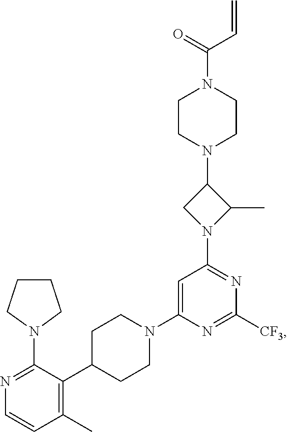 [SMILES: C=CC(=O)N1CCN(C2CN(c3cc(N4CCC(c5c(C)ccnc5N5CCCC5)CC4)nc(C(F)(F)F)n3)C2C)CC1]

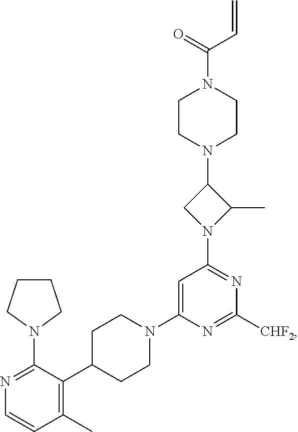 [SMILES: C=CC(=O)N1CCN(C2CN(c3cc(N4CCC(c5c(C)ccnc5N5CCCC5)CC4)nc(C(F)F)n3)C2C)CC1]

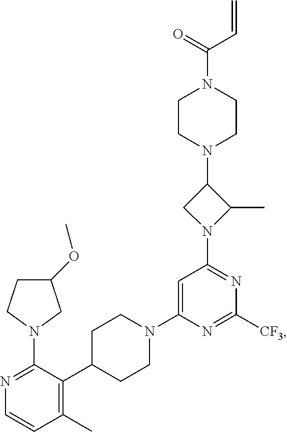 [SMILES: C=CC(=O)N1CCN(C2CN(c3cc(N4CCC(c5c(C)ccnc5N5CCC(OC)C5)CC4)nc(C(F)(F)F)n3)C2C)CC1]

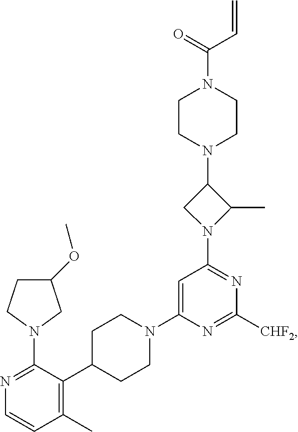 [SMILES: C=CC(=O)N1CCN(C2CN(c3cc(N4CCC(c5c(C)ccnc5N5CCC(OC)C5)CC4)nc(C(F)F)n3)C2C)CC1]

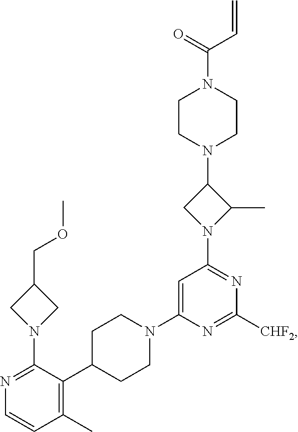 [SMILES: C=CC(=O)N1CCN(C2CN(c3cc(N4CCC(c5c(C)ccnc5N5CC(COC)C5)CC4)nc(C(F)F)n3)C2C)CC1]

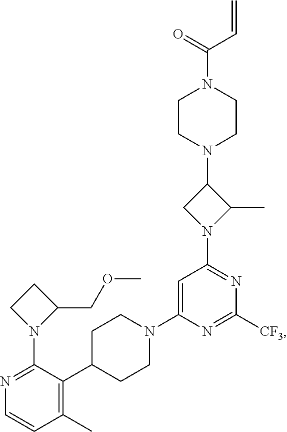 [SMILES: C=CC(=O)N1CCN(C2CN(c3cc(N4CCC(c5c(C)ccnc5N5CCC5COC)CC4)nc(C(F)(F)F)n3)C2C)CC1]

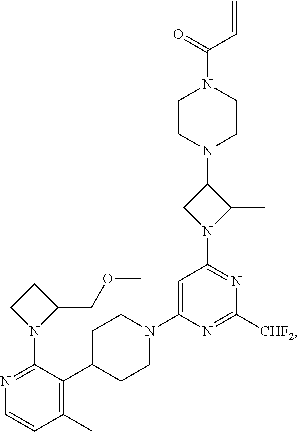 [SMILES: C=CC(=O)N1CCN(C2CN(c3cc(N4CCC(c5c(C)ccnc5N5CCC5COC)CC4)nc(C(F)F)n3)C2C)CC1]

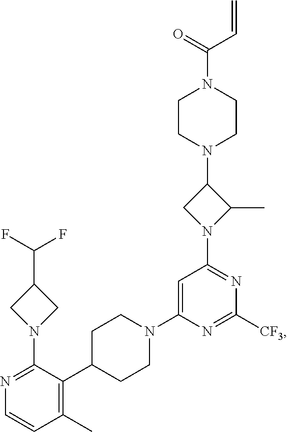 [SMILES: C=CC(=O)N1CCN(C2CN(c3cc(N4CCC(c5c(C)ccnc5N5CC(C(F)F)C5)CC4)nc(C(F)(F)F)n3)C2C)CC1]

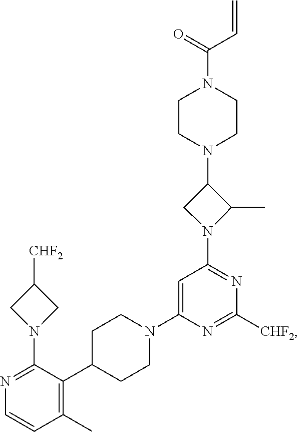 [SMILES: C=CC(=O)N1CCN(C2CN(c3cc(N4CCC(c5c(C)ccnc5N5CC(C(F)F)C5)CC4)nc(C(F)F)n3)C2C)CC1]

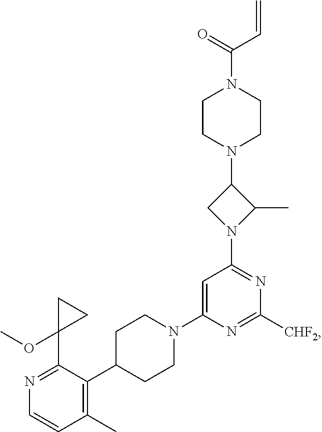 [SMILES: C=CC(=O)N1CCN(C2CN(c3cc(N4CCC(c5c(C)ccnc5C5(OC)CC5)CC4)nc(C(F)F)n3)C2C)CC1]

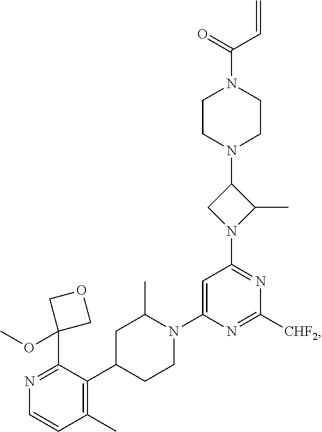 [SMILES: C=CC(=O)N1CCN(C2CN(c3cc(N4CCC(c5c(C)ccnc5C5(OC)COC5)CC4C)nc(C(F)F)n3)C2C)CC1]

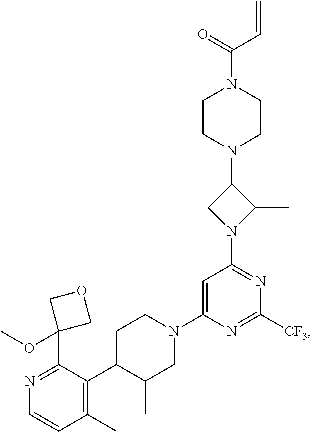 [SMILES: C=CC(=O)N1CCN(C2CN(c3cc(N4CCC(c5c(C)ccnc5C5(OC)COC5)C(C)C4)nc(C(F)(F)F)n3)C2C)CC1]

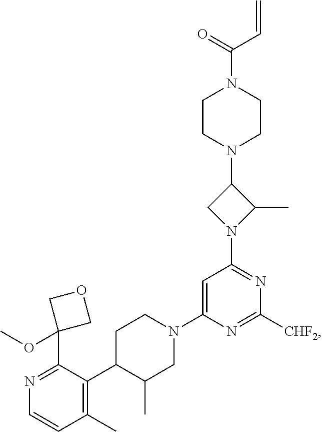 [SMILES: C=CC(=O)N1CCN(C2CN(c3cc(N4CCC(c5c(C)ccnc5C5(OC)COC5)C(C)C4)nc(C(F)F)n3)C2C)CC1]

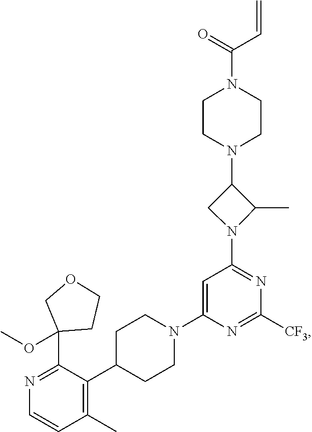 [SMILES: C=CC(=O)N1CCN(C2CN(c3cc(N4CCC(c5c(C)ccnc5C5(OC)CCOC5)CC4)nc(C(F)(F)F)n3)C2C)CC1]

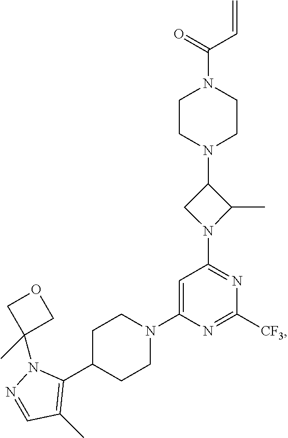 [SMILES: C=CC(=O)N1CCN(C2CN(c3cc(N4CCC(c5c(C)cnn5C5(C)COC5)CC4)nc(C(F)(F)F)n3)C2C)CC1]

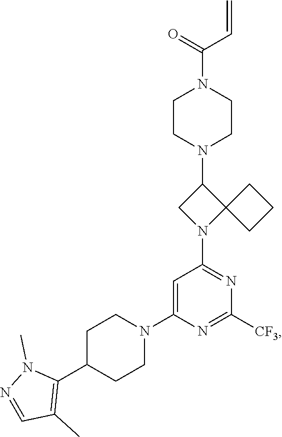 [SMILES: C=CC(=O)N1CCN(C2CN(c3cc(N4CCC(c5c(C)cnn5C)CC4)nc(C(F)(F)F)n3)C23CCC3)CC1]

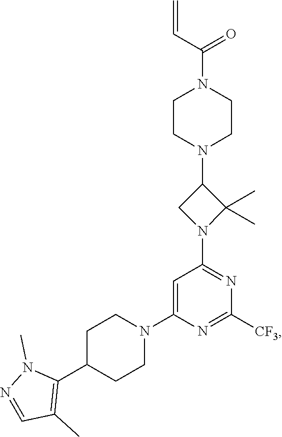 [SMILES: C=CC(=O)N1CCN(C2CN(c3cc(N4CCC(c5c(C)cnn5C)CC4)nc(C(F)(F)F)n3)C2(C)C)CC1]

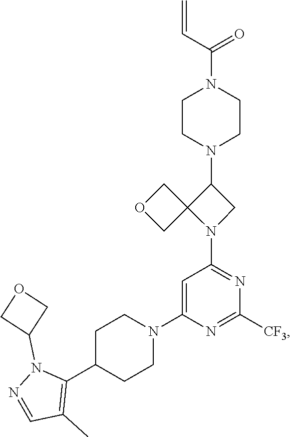 [SMILES: C=CC(=O)N1CCN(C2CN(c3cc(N4CCC(c5c(C)cnn5C5COC5)CC4)nc(C(F)(F)F)n3)C23COC3)CC1]

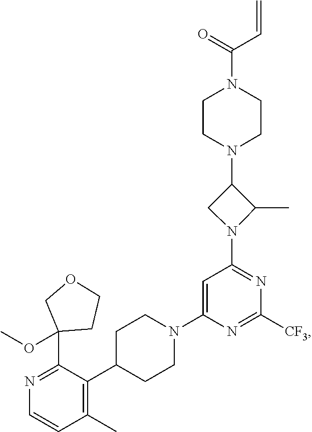 [SMILES: C=CC(=O)N1CCN(C2CN(c3cc(N4CCC(c5c(C)ccnc5C5(OC)CCOC5)CC4)nc(C(F)(F)F)n3)C2C)CC1]

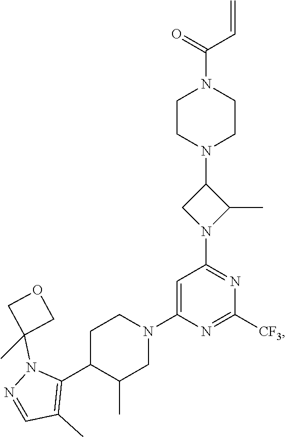 [SMILES: C=CC(=O)N1CCN(C2CN(c3cc(N4CCC(c5c(C)cnn5C5(C)COC5)C(C)C4)nc(C(F)(F)F)n3)C2C)CC1]

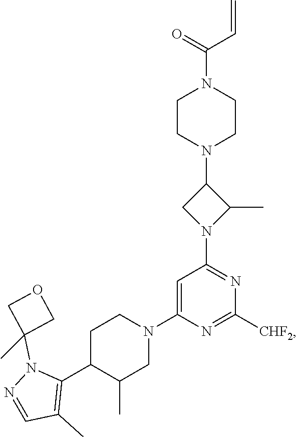 [SMILES: C=CC(=O)N1CCN(C2CN(c3cc(N4CCC(c5c(C)cnn5C5(C)COC5)C(C)C4)nc(C(F)F)n3)C2C)CC1]

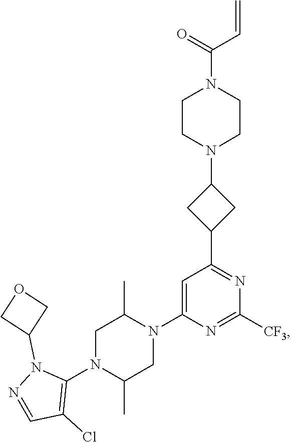 [SMILES: C=CC(=O)N1CCN(C2CC(c3cc(N4CC(C)N(c5c(Cl)cnn5C5COC5)CC4C)nc(C(F)(F)F)n3)C2)CC1]

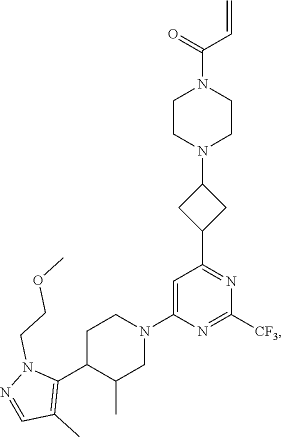 [SMILES: C=CC(=O)N1CCN(C2CC(c3cc(N4CCC(c5c(C)cnn5CCOC)C(C)C4)nc(C(F)(F)F)n3)C2)CC1]

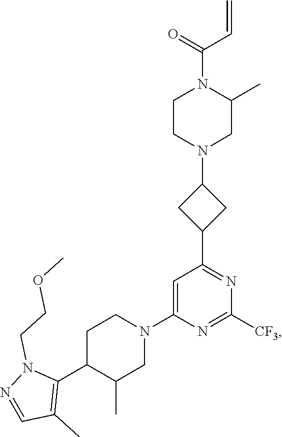 [SMILES: C=CC(=O)N1CCN(C2CC(c3cc(N4CCC(c5c(C)cnn5CCOC)C(C)C4)nc(C(F)(F)F)n3)C2)CC1C]

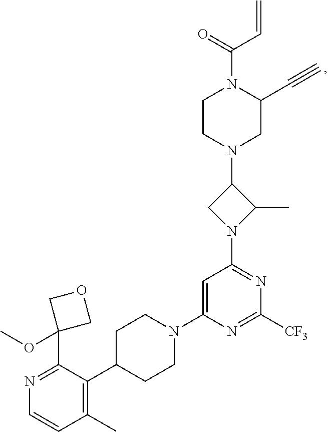 [SMILES: C#CC1CN(C2CN(c3cc(N4CCC(c5c(C)ccnc5C5(OC)COC5)CC4)nc(C(F)(F)F)n3)C2C)CCN1C(=O)C=C]

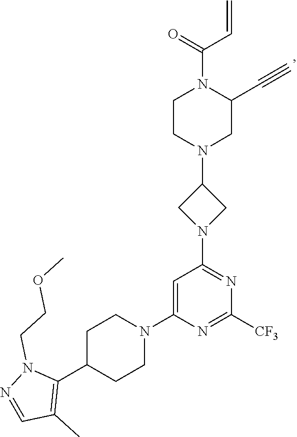 [SMILES: C#CC1CN(C2CN(c3cc(N4CCC(c5c(C)cnn5CCOC)CC4)nc(C(F)(F)F)n3)C2)CCN1C(=O)C=C]

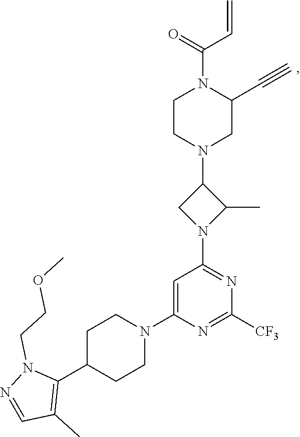 [SMILES: C#CC1CN(C2CN(c3cc(N4CCC(c5c(C)cnn5CCOC)CC4)nc(C(F)(F)F)n3)C2C)CCN1C(=O)C=C]

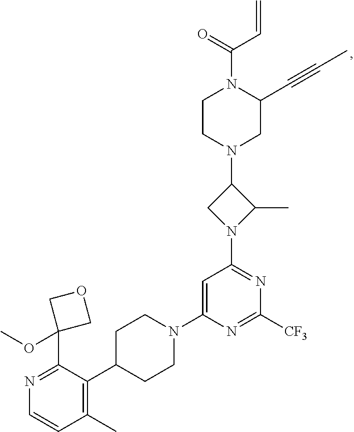 [SMILES: C=CC(=O)N1CCN(C2CN(c3cc(N4CCC(c5c(C)ccnc5C5(OC)COC5)CC4)nc(C(F)(F)F)n3)C2C)CC1C#CC]

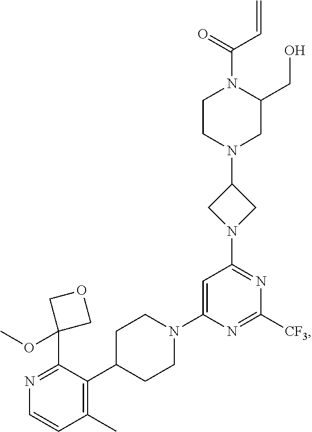 [SMILES: C=CC(=O)N1CCN(C2CN(c3cc(N4CCC(c5c(C)ccnc5C5(OC)COC5)CC4)nc(C(F)(F)F)n3)C2)CC1CO]

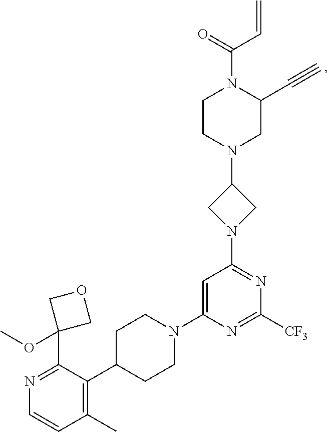 [SMILES: C#CC1CN(C2CN(c3cc(N4CCC(c5c(C)ccnc5C5(OC)COC5)CC4)nc(C(F)(F)F)n3)C2)CCN1C(=O)C=C]

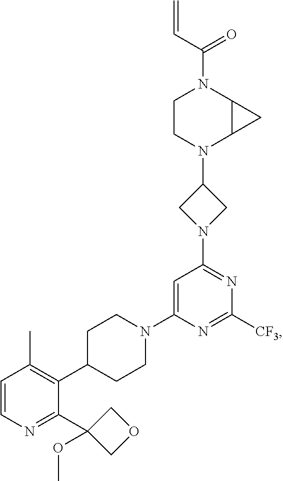 [SMILES: C=CC(=O)N1CCN(C2CN(c3cc(N4CCC(c5c(C)ccnc5C5(OC)COC5)CC4)nc(C(F)(F)F)n3)C2)C2CC21]

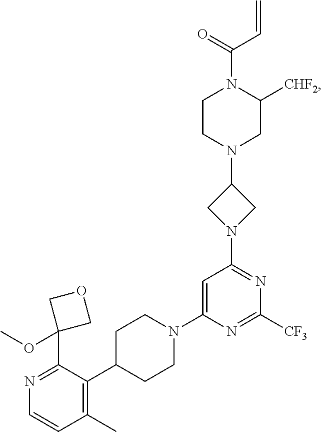 [SMILES: C=CC(=O)N1CCN(C2CN(c3cc(N4CCC(c5c(C)ccnc5C5(OC)COC5)CC4)nc(C(F)(F)F)n3)C2)CC1C(F)F]

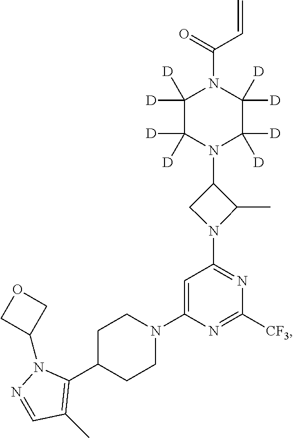 [SMILES: [2H]C1([2H])N(C(=O)C=C)C([2H])([2H])C([2H])([2H])N(C2CN(c3cc(N4CCC(c5c(C)cnn5C5COC5)CC4)nc(C(F)(F)F)n3)C2C)C1([2H])[2H]]

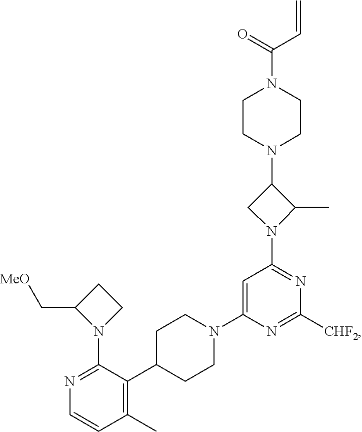 [SMILES: C=CC(=O)N1CCN(C2CN(c3cc(N4CCC(c5c(C)ccnc5N5CCC5COC)CC4)nc(C(F)F)n3)C2C)CC1]

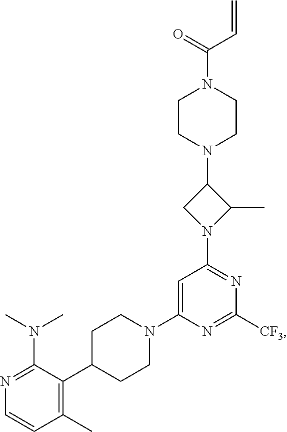 [SMILES: C=CC(=O)N1CCN(C2CN(c3cc(N4CCC(c5c(C)ccnc5N(C)C)CC4)nc(C(F)(F)F)n3)C2C)CC1]

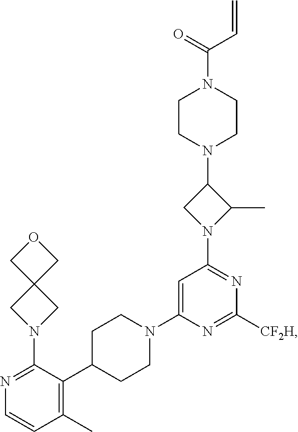 [SMILES: C=CC(=O)N1CCN(C2CN(c3cc(N4CCC(c5c(C)ccnc5N5CC6(COC6)C5)CC4)nc(C(F)F)n3)C2C)CC1]

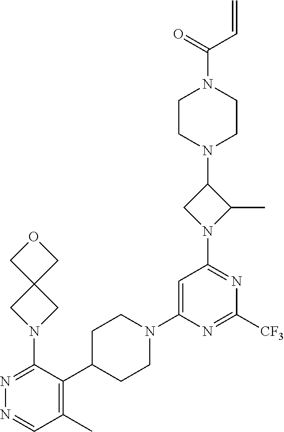 [SMILES: C=CC(=O)N1CCN(C2CN(c3cc(N4CCC(c5c(C)cnnc5N5CC6(COC6)C5)CC4)nc(C(F)(F)F)n3)C2C)CC1]

In some cases, the compound of Formula (I) is selected from the list of compounds in Table A, or a pharmaceutically acceptable salt thereof.

In some cases, A is CH, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy; and X is

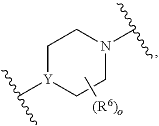

and the disclosure provides compounds of Formula (IA):

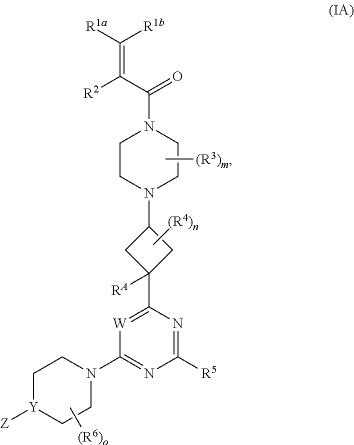

and pharmaceutically acceptable salts thereof, wherein R^{A} is H, halo, CN, C₁₋₃ alkyl, C₁₋₃ haloalkyl, C₀₋₃ alkyleneOH, or C₀₋₃ alkylene-C₁₋₄ alkoxy; and the remaining substituents are as previously defined herein, Contemplated compounds of Formula (IA) include but are not limited to:

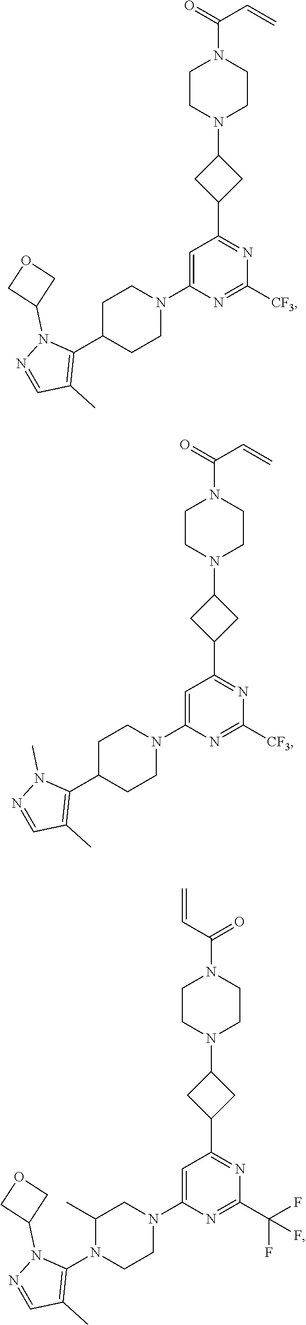 [SMILES: C=CC(=O)N1CCN(C2CC(c3cc(N4CCC(c5c(C)cnn5C5COC5)CC4)nc(C(F)(F)F)n3)C2)CC1 ; C=CC(=O)N1CCN(C2CC(c3cc(N4CCC(c5c(C)cnn5C)CC4)nc(C(F)(F)F)n3)C2)CC1 ; C=CC(=O)N1CCN(C2CC(c3cc(N4CCN(c5c(C)cnn5C5COC5)C(C)C4)nc(C(F)(F)F)n3)C2)CC1]

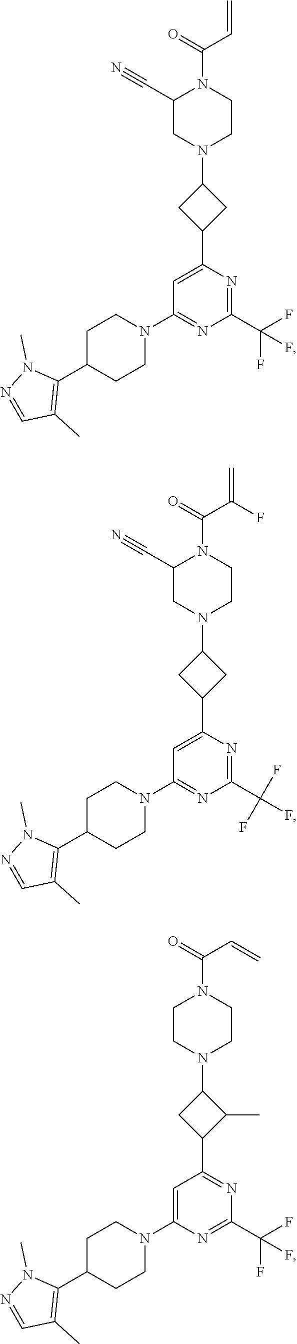 [SMILES: C=CC(=O)N1CCN(C2CC(c3cc(N4CCC(c5c(C)cnn5C)CC4)nc(C(F)(F)F)n3)C2)CC1C#N ; C=C(F)C(=O)N1CCN(C2CC(c3cc(N4CCC(c5c(C)cnn5C)CC4)nc(C(F)(F)F)n3)C2)CC1C#N ; C=CC(=O)N1CCN(C2CC(c3cc(N4CCC(c5c(C)cnn5C)CC4)nc(C(F)(F)F)n3)C2C)CC1]

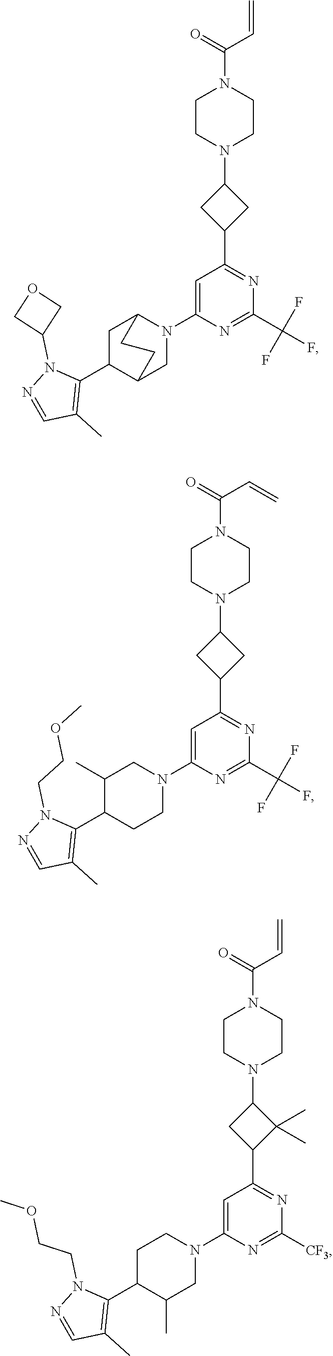 [SMILES: C=CC(=O)N1CCN(C2CC(c3cc(N4CC5CCC4CC5c4c(C)cnn4C4COC4)nc(C(F)(F)F)n3)C2)CC1 ; C=CC(=O)N1CCN(C2CC(c3cc(N4CCC(c5c(C)cnn5CCOC)C(C)C4)nc(C(F)(F)F)n3)C2)CC1 ; C=CC(=O)N1CCN(C2CC(c3cc(N4CCC(c5c(C)cnn5CCOC)C(C)C4)nc(C(F)(F)F)n3)C2(C)C)CC1]

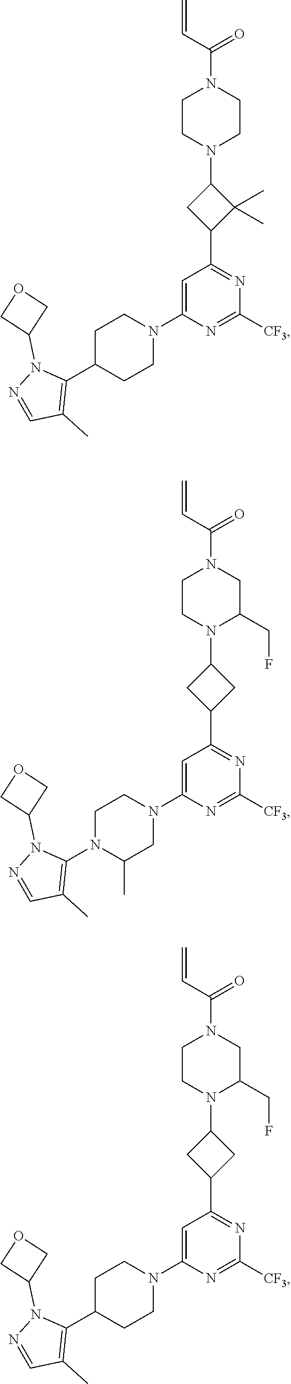 [SMILES: C=CC(=O)N1CCN(C2CC(c3cc(N4CCC(c5c(C)cnn5C5COC5)CC4)nc(C(F)(F)F)n3)C2(C)C)CC1 ; C=CC(=O)N1CCN(C2CC(c3cc(N4CCN(c5c(C)cnn5C5COC5)C(C)C4)nc(C(F)(F)F)n3)C2)C(CF)C1 ; C=CC(=O)N1CCN(C2CC(c3cc(N4CCC(c5c(C)cnn5C5COC5)CC4)nc(C(F)(F)F)n3)C2)C(CF)C1]

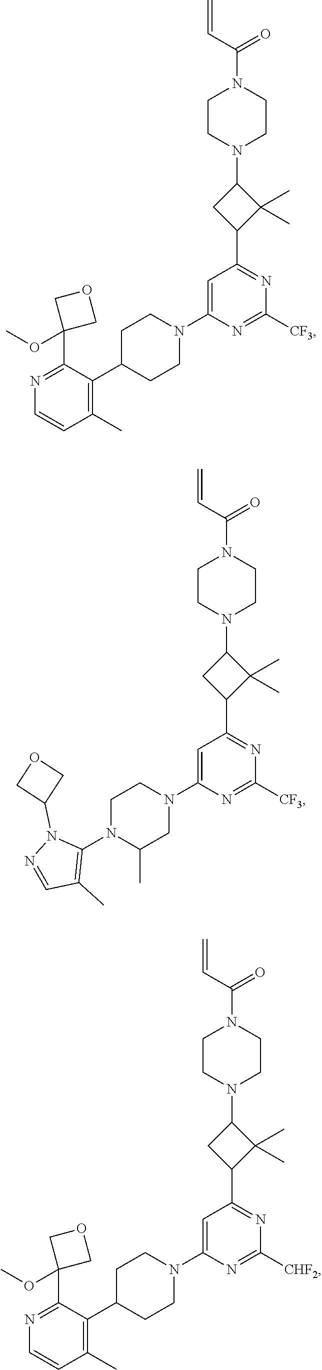 [SMILES: C=CC(=O)N1CCN(C2CC(c3cc(N4CCC(c5c(C)ccnc5C5(OC)COC5)CC4)nc(C(F)(F)F)n3)C2(C)C)CC1 ; C=CC(=O)N1CCN(C2CC(c3cc(N4CCN(c5c(C)cnn5C5COC5)C(C)C4)nc(C(F)(F)F)n3)C2(C)C)CC1 ; C=CC(=O)N1CCN(C2CC(c3cc(N4CCC(c5c(C)ccnc5C5(OC)COC5)CC4)nc(C(F)F)n3)C2(C)C)CC1]

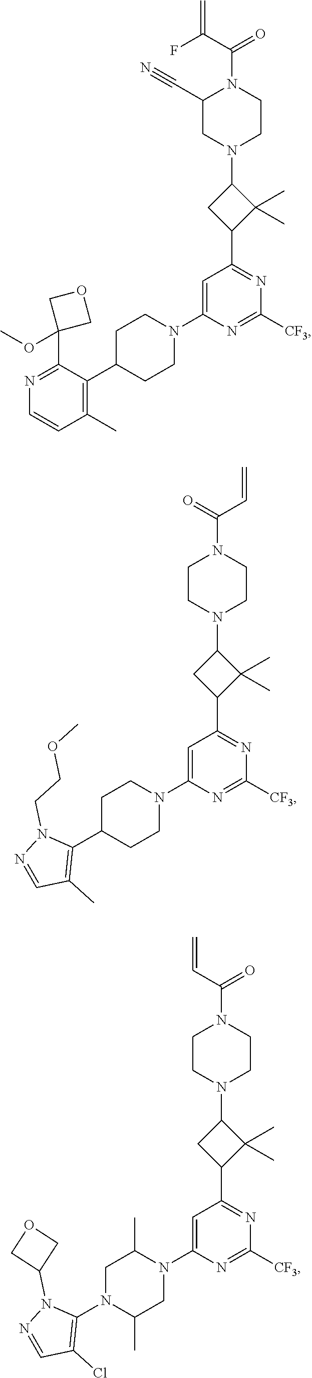 [SMILES: C=C(F)C(=O)N1CCN(C2CC(c3cc(N4CCC(c5c(C)ccnc5C5(OC)COC5)CC4)nc(C(F)(F)F)n3)C2(C)C)CC1C#N ; C=CC(=O)N1CCN(C2CC(c3cc(N4CCC(c5c(C)cnn5CCOC)CC4)nc(C(F)(F)F)n3)C2(C)C)CC1 ; C=CC(=O)N1CCN(C2CC(c3cc(N4CC(C)N(c5c(Cl)cnn5C5COC5)CC4C)nc(C(F)(F)F)n3)C2(C)C)CC1]

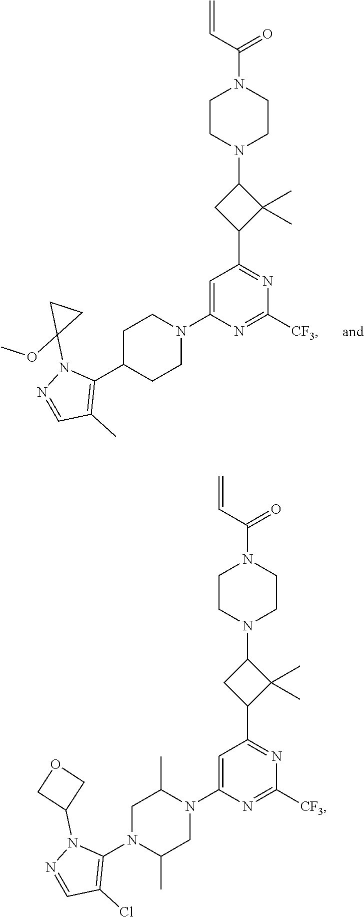 [SMILES: C=CC(=O)N1CCN(C2CC(c3cc(N4CCC(c5c(C)cnn5C5(OC)CC5)CC4)nc(C(F)(F)F)n3)C2(C)C)CC1 ; C=CC(=O)N1CCN(C2CC(c3cc(N4CC(C)N(c5c(Cl)cnn5C5COC5)CC4C)nc(C(F)(F)F)n3)C2(C)C)CC1]

and pharmaceutically acceptable salts thereof.

In some cases, A is N and X is

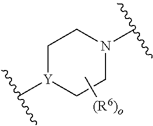

and the disclosure provides compounds of Formula (IB):

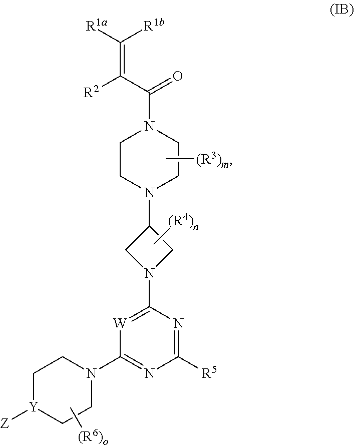

and pharmaceutically acceptable salts thereof, wherein the substituents are as previously defined herein, Contemplated compounds of Formula (TB) include, but are not limited to, those listed in Table B, below, and pharmaceutically acceptable salts thereof.

TABLE B

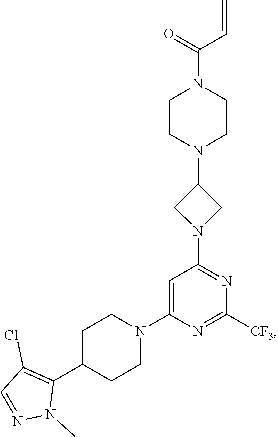 [SMILES: C=CC(=O)N1CCN(C2CN(c3cc(N4CCC(c5c(Cl)cnn5C)CC4)nc(C(F)(F)F)n3)C2)CC1]

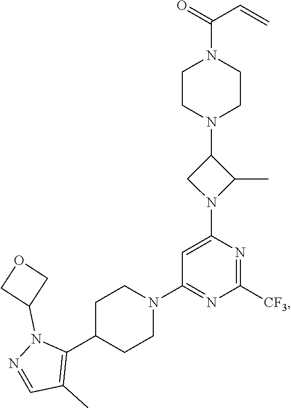 [SMILES: C=CC(=O)N1CCN(C2CN(c3cc(N4CCC(c5c(C)cnn5C5COC5)CC4)nc(C(F)(F)F)n3)C2C)CC1]

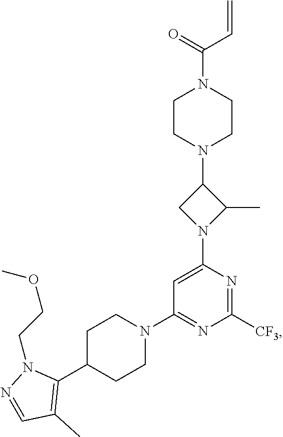 [SMILES: C=CC(=O)N1CCN(C2CN(c3cc(N4CCC(c5c(C)cnn5CCOC)CC4)nc(C(F)(F)F)n3)C2C)CC1]

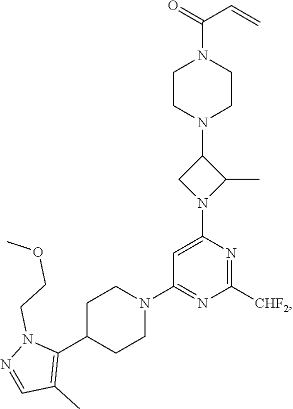 [SMILES: C=CC(=O)N1CCN(C2CN(c3cc(N4CCC(c5c(C)cnn5CCOC)CC4)nc(C(F)F)n3)C2C)CC1]

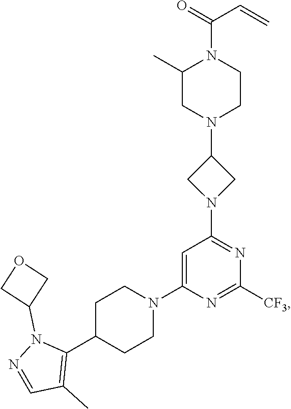 [SMILES: C=CC(=O)N1CCN(C2CN(c3cc(N4CCC(c5c(C)cnn5C5COC5)CC4)nc(C(F)(F)F)n3)C2)CC1C]

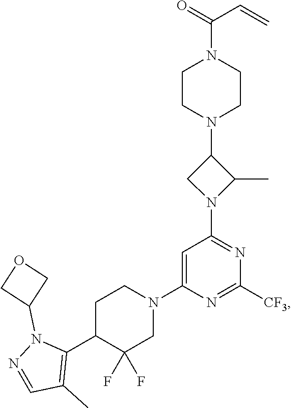 [SMILES: C=CC(=O)N1CCN(C2CN(c3cc(N4CCC(c5c(C)cnn5C5COC5)C(F)(F)C4)nc(C(F)(F)F)n3)C2C)CC1]

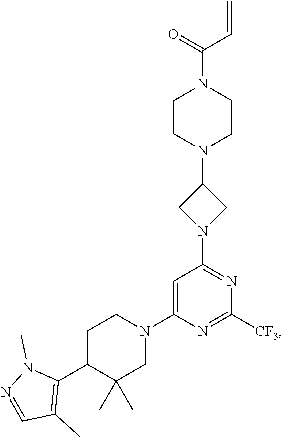 [SMILES: C=CC(=O)N1CCN(C2CN(c3cc(N4CCC(c5c(C)cnn5C)C(C)(C)C4)nc(C(F)(F)F)n3)C2)CC1]

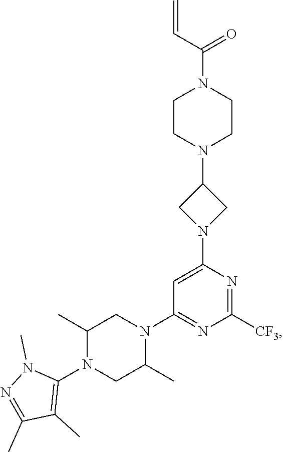 [SMILES: C=CC(=O)N1CCN(C2CN(c3cc(N4CC(C)N(c5c(C)c(C)nn5C)CC4C)nc(C(F)(F)F)n3)C2)CC1]

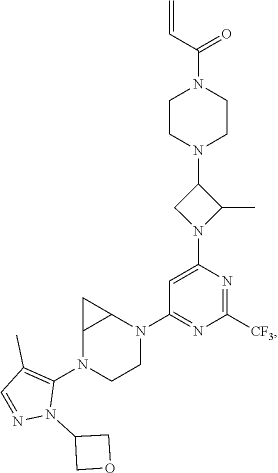 [SMILES: C=CC(=O)N1CCN(C2CN(c3cc(N4CCN(c5c(C)cnn5C5COC5)C5CC54)nc(C(F)(F)F)n3)C2C)CC1]

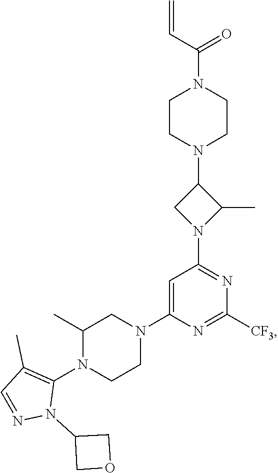 [SMILES: C=CC(=O)N1CCN(C2CN(c3cc(N4CCN(c5c(C)cnn5C5COC5)C(C)C4)nc(C(F)(F)F)n3)C2C)CC1]

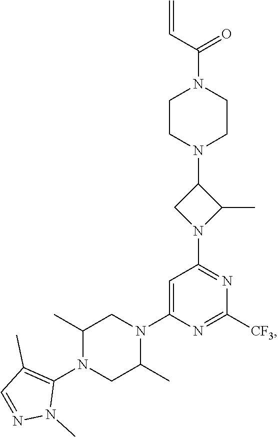 [SMILES: C=CC(=O)N1CCN(C2CN(c3cc(N4CC(C)N(c5c(C)cnn5C)CC4C)nc(C(F)(F)F)n3)C2C)CC1]

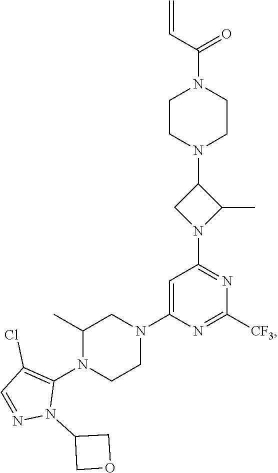 [SMILES: C=CC(=O)N1CCN(C2CN(c3cc(N4CCN(c5c(Cl)cnn5C5COC5)C(C)C4)nc(C(F)(F)F)n3)C2C)CC1]

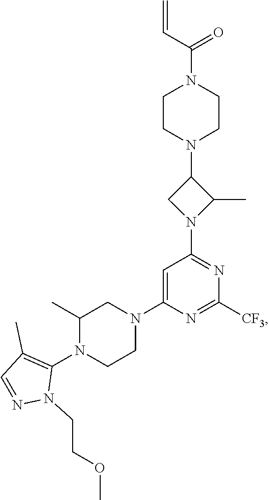 [SMILES: C=CC(=O)N1CCN(C2CN(c3cc(N4CCN(c5c(C)cnn5CCOC)C(C)C4)nc(C(F)(F)F)n3)C2C)CC1]

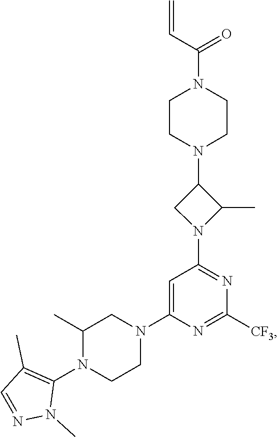 [SMILES: C=CC(=O)N1CCN(C2CN(c3cc(N4CCN(c5c(C)cnn5C)C(C)C4)nc(C(F)(F)F)n3)C2C)CC1]

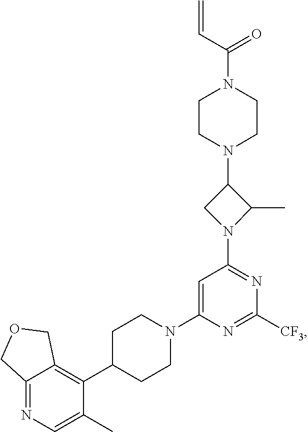 [SMILES: C=CC(=O)N1CCN(C2CN(c3cc(N4CCC(c5c(C)cnc6c5COC6)CC4)nc(C(F)(F)F)n3)C2C)CC1]

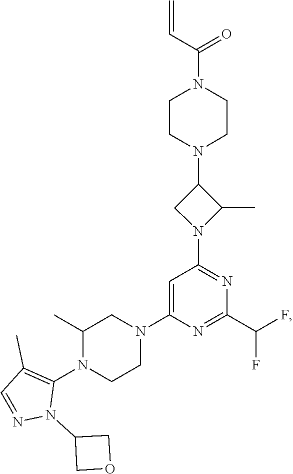 [SMILES: C=CC(=O)N1CCN(C2CN(c3cc(N4CCN(c5c(C)cnn5C5COC5)C(C)C4)nc(C(F)F)n3)C2C)CC1]

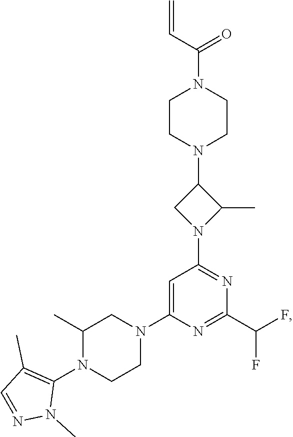 [SMILES: C=CC(=O)N1CCN(C2CN(c3cc(N4CCN(c5c(C)cnn5C)C(C)C4)nc(C(F)F)n3)C2C)CC1]

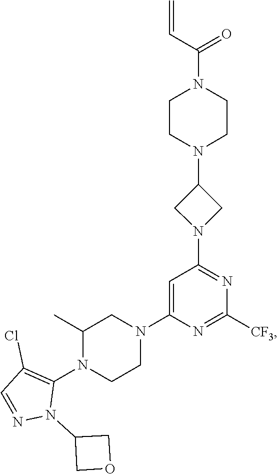 [SMILES: C=CC(=O)N1CCN(C2CN(c3cc(N4CCN(c5c(Cl)cnn5C5COC5)C(C)C4)nc(C(F)(F)F)n3)C2)CC1]

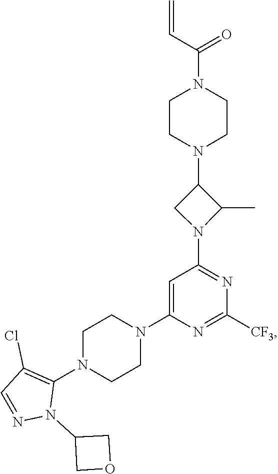 [SMILES: C=CC(=O)N1CCN(C2CN(c3cc(N4CCN(c5c(Cl)cnn5C5COC5)CC4)nc(C(F)(F)F)n3)C2C)CC1]

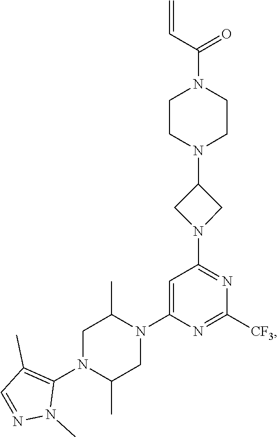 [SMILES: C=CC(=O)N1CCN(C2CN(c3cc(N4CC(C)N(c5c(C)cnn5C)CC4C)nc(C(F)(F)F)n3)C2)CC1]

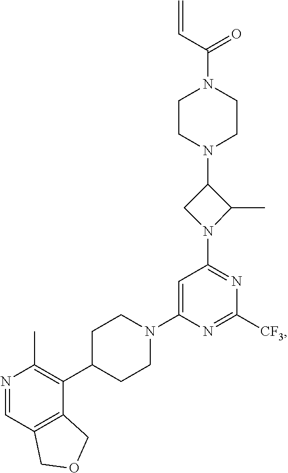 [SMILES: C=CC(=O)N1CCN(C2CN(c3cc(N4CCC(c5c(C)ncc6c5COC6)CC4)nc(C(F)(F)F)n3)C2C)CC1]

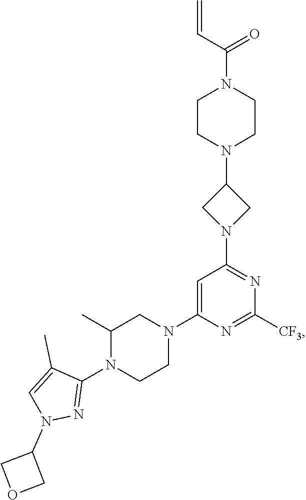 [SMILES: C=CC(=O)N1CCN(C2CN(c3cc(N4CCN(c5nn(C6COC6)cc5C)C(C)C4)nc(C(F)(F)F)n3)C2)CC1]

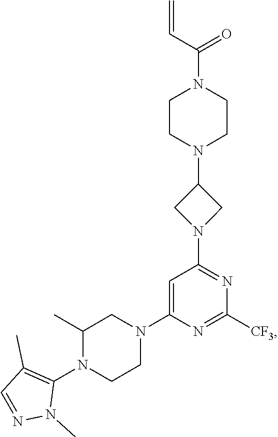 [SMILES: C=CC(=O)N1CCN(C2CN(c3cc(N4CCN(c5c(C)cnn5C)C(C)C4)nc(C(F)(F)F)n3)C2)CC1]

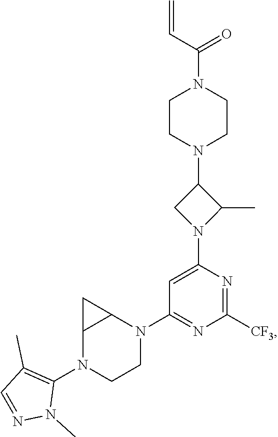 [SMILES: C=CC(=O)N1CCN(C2CN(c3cc(N4CCN(c5c(C)cnn5C)C5CC54)nc(C(F)(F)F)n3)C2C)CC1]

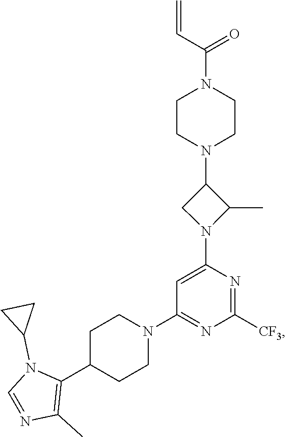 [SMILES: C=CC(=O)N1CCN(C2CN(c3cc(N4CCC(c5c(C)ncn5C5CC5)CC4)nc(C(F)(F)F)n3)C2C)CC1]

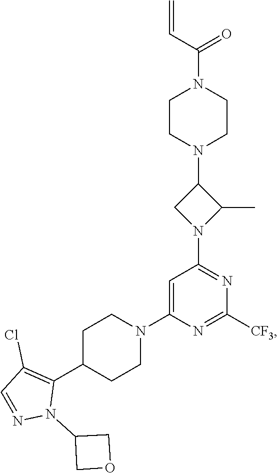 [SMILES: C=CC(=O)N1CCN(C2CN(c3cc(N4CCC(c5c(Cl)cnn5C5COC5)CC4)nc(C(F)(F)F)n3)C2C)CC1]

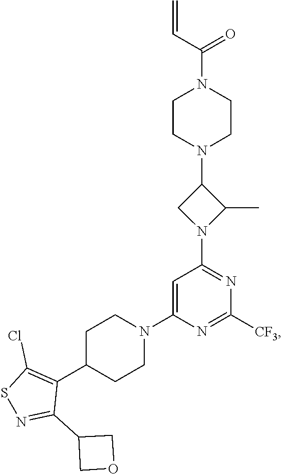 [SMILES: C=CC(=O)N1CCN(C2CN(c3cc(N4CCC(c5c(C6COC6)nsc5Cl)CC4)nc(C(F)(F)F)n3)C2C)CC1]

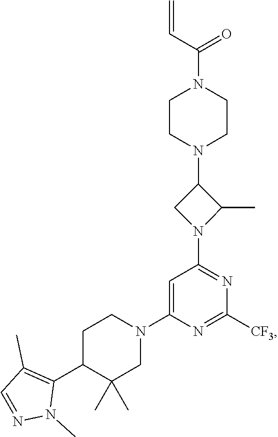 [SMILES: C=CC(=O)N1CCN(C2CN(c3cc(N4CCC(c5c(C)cnn5C)C(C)(C)C4)nc(C(F)(F)F)n3)C2C)CC1]

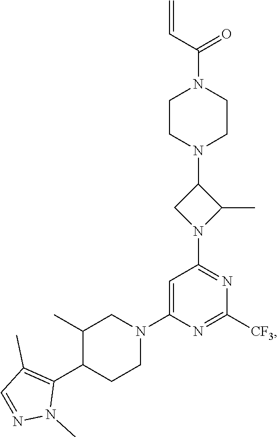 [SMILES: C=CC(=O)N1CCN(C2CN(c3cc(N4CCC(c5c(C)cnn5C)C(C)C4)nc(C(F)(F)F)n3)C2C)CC1]

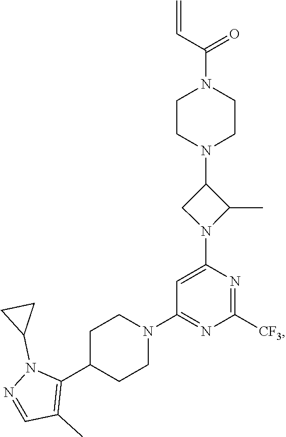 [SMILES: C=CC(=O)N1CCN(C2CN(c3cc(N4CCC(c5c(C)cnn5C5CC5)CC4)nc(C(F)(F)F)n3)C2C)CC1]

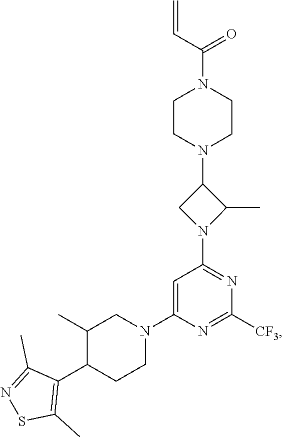 [SMILES: C=CC(=O)N1CCN(C2CN(c3cc(N4CCC(c5c(C)nsc5C)C(C)C4)nc(C(F)(F)F)n3)C2C)CC1]

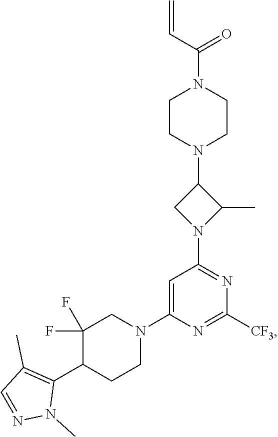 [SMILES: C=CC(=O)N1CCN(C2CN(c3cc(N4CCC(c5c(C)cnn5C)C(F)(F)C4)nc(C(F)(F)F)n3)C2C)CC1]

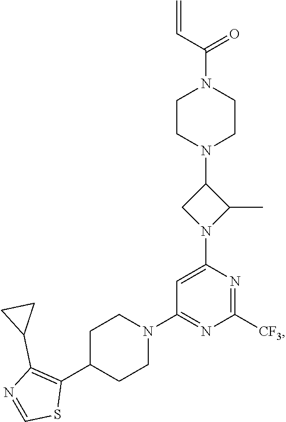 [SMILES: C=CC(=O)N1CCN(C2CN(c3cc(N4CCC(c5scnc5C5CC5)CC4)nc(C(F)(F)F)n3)C2C)CC1]

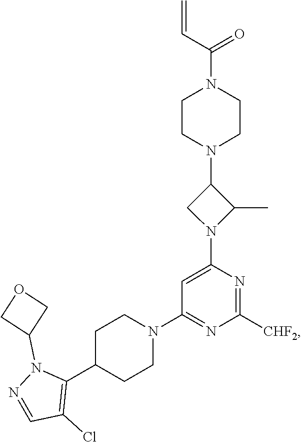 [SMILES: C=CC(=O)N1CCN(C2CN(c3cc(N4CCC(c5c(Cl)cnn5C5COC5)CC4)nc(C(F)F)n3)C2C)CC1]

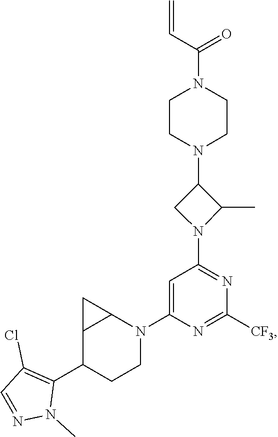 [SMILES: C=CC(=O)N1CCN(C2CN(c3cc(N4CCC(c5c(Cl)cnn5C)C5CC54)nc(C(F)(F)F)n3)C2C)CC1]

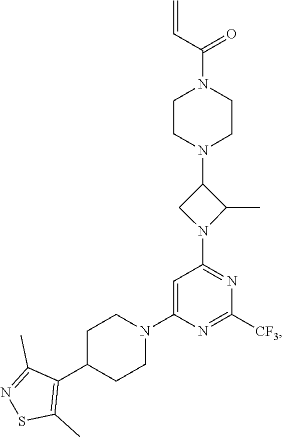 [SMILES: C=CC(=O)N1CCN(C2CN(c3cc(N4CCC(c5c(C)nsc5C)CC4)nc(C(F)(F)F)n3)C2C)CC1]

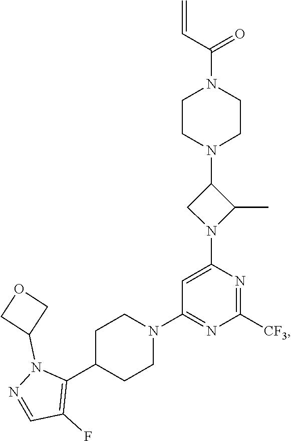 [SMILES: C=CC(=O)N1CCN(C2CN(c3cc(N4CCC(c5c(F)cnn5C5COC5)CC4)nc(C(F)(F)F)n3)C2C)CC1]

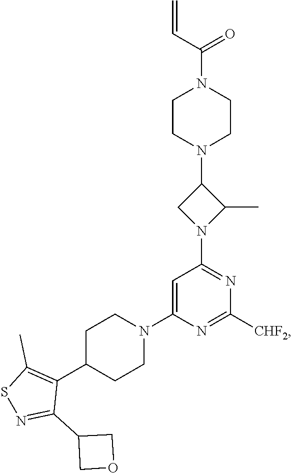 [SMILES: C=CC(=O)N1CCN(C2CN(c3cc(N4CCC(c5c(C6COC6)nsc5C)CC4)nc(C(F)F)n3)C2C)CC1]

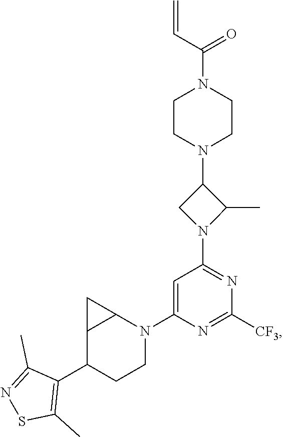 [SMILES: C=CC(=O)N1CCN(C2CN(c3cc(N4CCC(c5c(C)nsc5C)C5CC54)nc(C(F)(F)F)n3)C2C)CC1]

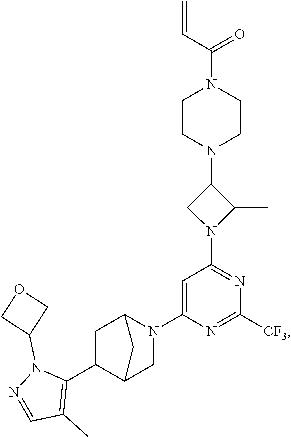 [SMILES: C=CC(=O)N1CCN(C2CN(c3cc(N4CC5CC4CC5c4c(C)cnn4C4COC4)nc(C(F)(F)F)n3)C2C)CC1]

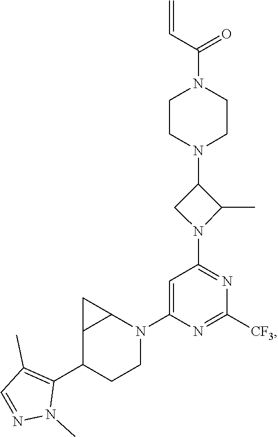 [SMILES: C=CC(=O)N1CCN(C2CN(c3cc(N4CCC(c5c(C)cnn5C)C5CC54)nc(C(F)(F)F)n3)C2C)CC1]

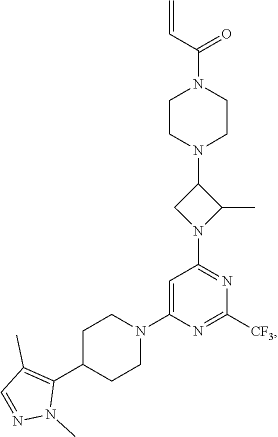 [SMILES: C=CC(=O)N1CCN(C2CN(c3cc(N4CCC(c5c(C)cnn5C)CC4)nc(C(F)(F)F)n3)C2C)CC1]

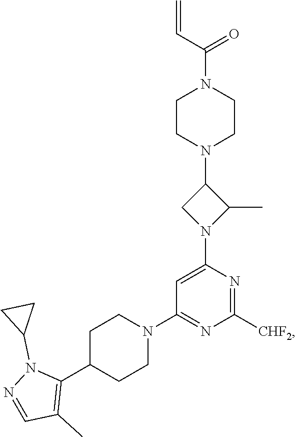 [SMILES: C=CC(=O)N1CCN(C2CN(c3cc(N4CCC(c5c(C)cnn5C5CC5)CC4)nc(C(F)F)n3)C2C)CC1]

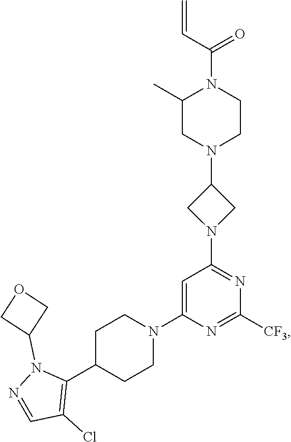 [SMILES: C=CC(=O)N1CCN(C2CN(c3cc(N4CCC(c5c(Cl)cnn5C5COC5)CC4)nc(C(F)(F)F)n3)C2)CC1C]

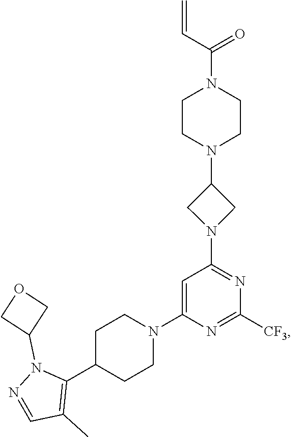 [SMILES: C=CC(=O)N1CCN(C2CN(c3cc(N4CCC(c5c(C)cnn5C5COC5)CC4)nc(C(F)(F)F)n3)C2)CC1]

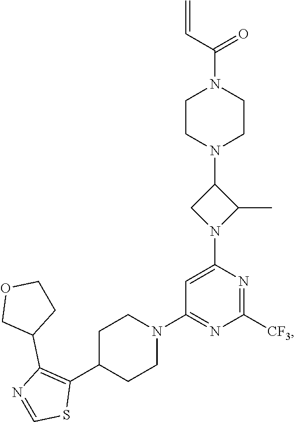 [SMILES: C=CC(=O)N1CCN(C2CN(c3cc(N4CCC(c5scnc5C5CCOC5)CC4)nc(C(F)(F)F)n3)C2C)CC1]

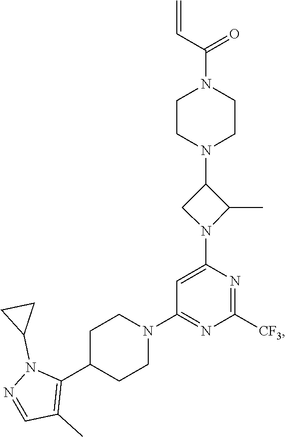 [SMILES: C=CC(=O)N1CCN(C2CN(c3cc(N4CCC(c5c(C)cnn5C5CC5)CC4)nc(C(F)(F)F)n3)C2C)CC1]

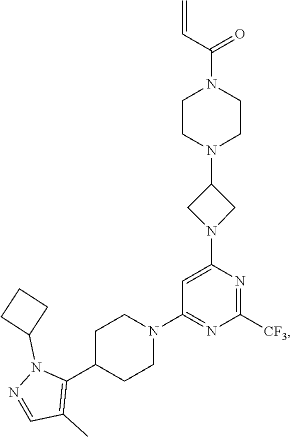 [SMILES: C=CC(=O)N1CCN(C2CN(c3cc(N4CCC(c5c(C)cnn5C5CCC5)CC4)nc(C(F)(F)F)n3)C2)CC1]

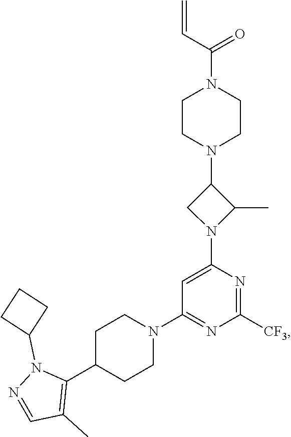 [SMILES: C=CC(=O)N1CCN(C2CN(c3cc(N4CCC(c5c(C)cnn5C5CCC5)CC4)nc(C(F)(F)F)n3)C2C)CC1]

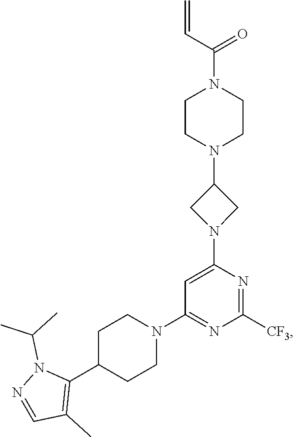 [SMILES: C=CC(=O)N1CCN(C2CN(c3cc(N4CCC(c5c(C)cnn5C(C)C)CC4)nc(C(F)(F)F)n3)C2)CC1]

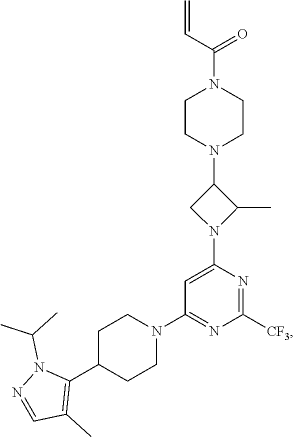 [SMILES: C=CC(=O)N1CCN(C2CN(c3cc(N4CCC(c5c(C)cnn5C(C)C)CC4)nc(C(F)(F)F)n3)C2C)CC1]

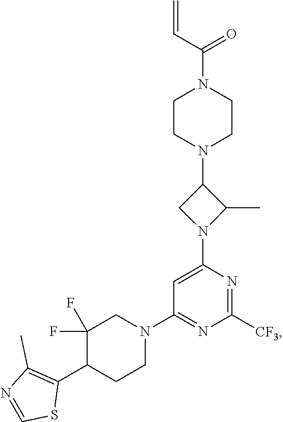 [SMILES: C=CC(=O)N1CCN(C2CN(c3cc(N4CCC(c5scnc5C)C(F)(F)C4)nc(C(F)(F)F)n3)C2C)CC1]

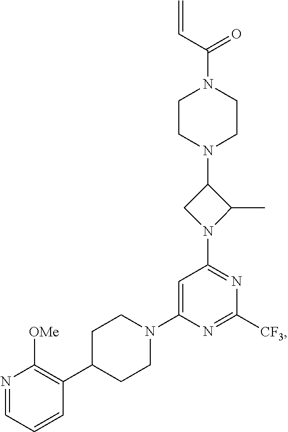 [SMILES: C=CC(=O)N1CCN(C2CN(c3cc(N4CCC(c5cccnc5OC)CC4)nc(C(F)(F)F)n3)C2C)CC1]

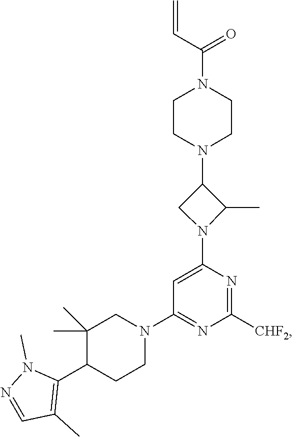 [SMILES: C=CC(=O)N1CCN(C2CN(c3cc(N4CCC(c5c(C)cnn5C)C(C)(C)C4)nc(C(F)F)n3)C2C)CC1]

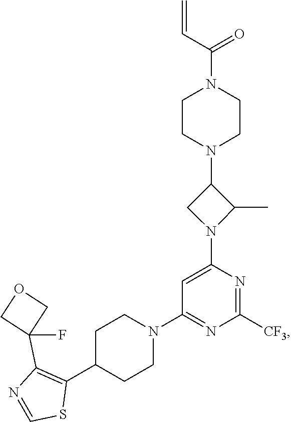 [SMILES: C=CC(=O)N1CCN(C2CN(c3cc(N4CCC(c5scnc5C5(F)COC5)CC4)nc(C(F)(F)F)n3)C2C)CC1]

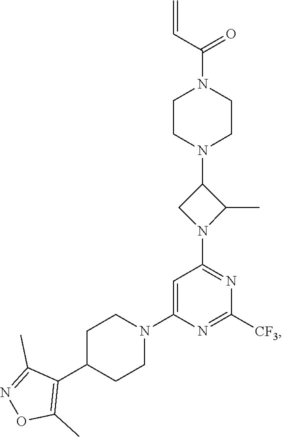 [SMILES: C=CC(=O)N1CCN(C2CN(c3cc(N4CCC(c5c(C)noc5C)CC4)nc(C(F)(F)F)n3)C2C)CC1]

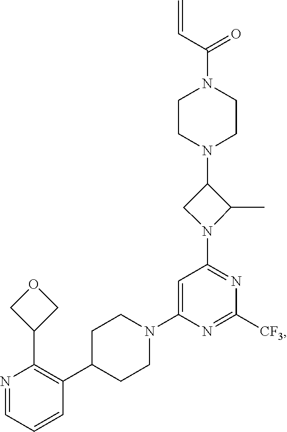 [SMILES: C=CC(=O)N1CCN(C2CN(c3cc(N4CCC(c5cccnc5C5COC5)CC4)nc(C(F)(F)F)n3)C2C)CC1]

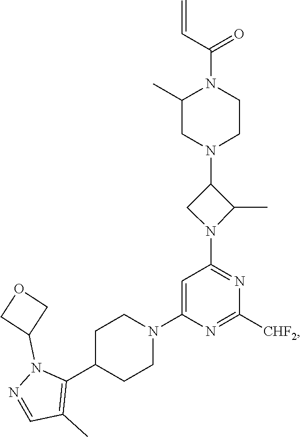 [SMILES: C=CC(=O)N1CCN(C2CN(c3cc(N4CCC(c5c(C)cnn5C5COC5)CC4)nc(C(F)F)n3)C2C)CC1C]

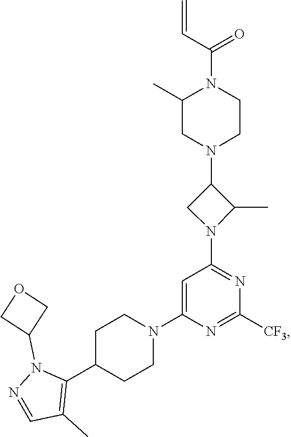 [SMILES: C=CC(=O)N1CCN(C2CN(c3cc(N4CCC(c5c(C)cnn5C5COC5)CC4)nc(C(F)(F)F)n3)C2C)CC1C]

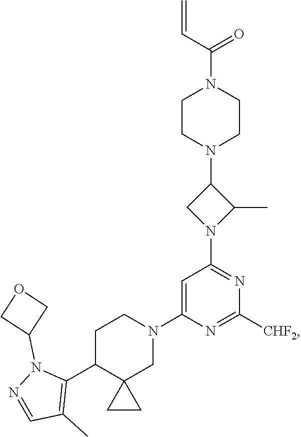 [SMILES: C=CC(=O)N1CCN(C2CN(c3cc(N4CCC(c5c(C)cnn5C5COC5)C5(CC5)C4)nc(C(F)F)n3)C2C)CC1]

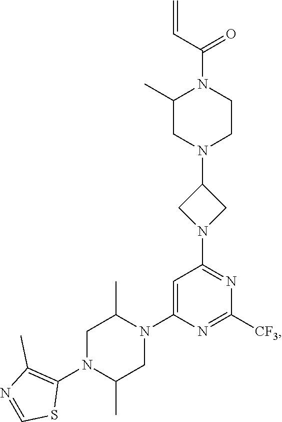 [SMILES: C=CC(=O)N1CCN(C2CN(c3cc(N4CC(C)N(c5scnc5C)CC4C)nc(C(F)(F)F)n3)C2)CC1C]

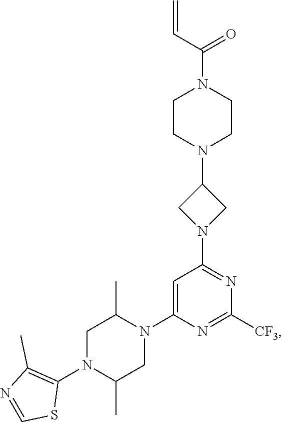 [SMILES: C=CC(=O)N1CCN(C2CN(c3cc(N4CC(C)N(c5scnc5C)CC4C)nc(C(F)(F)F)n3)C2)CC1]

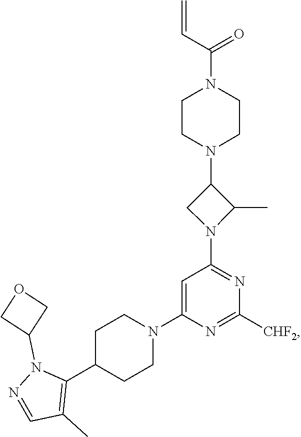 [SMILES: C=CC(=O)N1CCN(C2CN(c3cc(N4CCC(c5c(C)cnn5C5COC5)CC4)nc(C(F)F)n3)C2C)CC1]

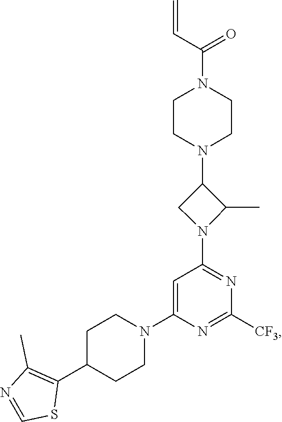 [SMILES: C=CC(=O)N1CCN(C2CN(c3cc(N4CCC(c5scnc5C)CC4)nc(C(F)(F)F)n3)C2C)CC1]

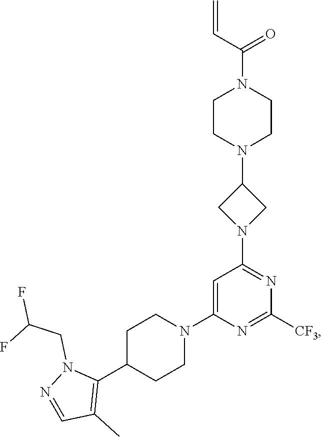 [SMILES: C=CC(=O)N1CCN(C2CN(c3cc(N4CCC(c5c(C)cnn5CC(F)F)CC4)nc(C(F)(F)F)n3)C2)CC1]

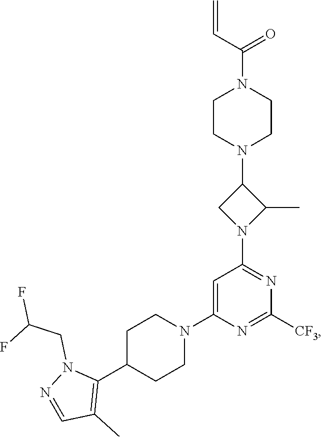 [SMILES: C=CC(=O)N1CCN(C2CN(c3cc(N4CCC(c5c(C)cnn5CC(F)F)CC4)nc(C(F)(F)F)n3)C2C)CC1]

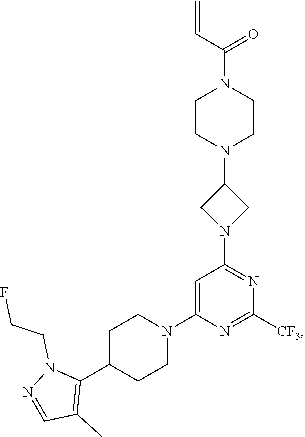 [SMILES: C=CC(=O)N1CCN(C2CN(c3cc(N4CCC(c5c(C)cnn5CCF)CC4)nc(C(F)(F)F)n3)C2)CC1]

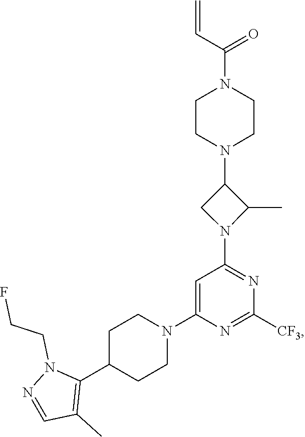 [SMILES: C=CC(=O)N1CCN(C2CN(c3cc(N4CCC(c5c(C)cnn5CCF)CC4)nc(C(F)(F)F)n3)C2C)CC1]

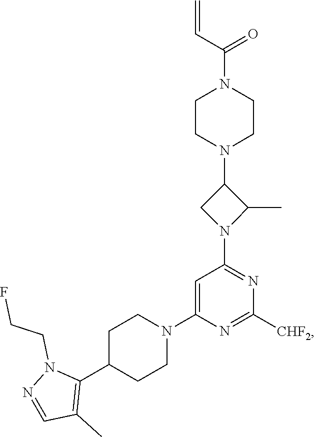 [SMILES: C=CC(=O)N1CCN(C2CN(c3cc(N4CCC(c5c(C)cnn5CCF)CC4)nc(C(F)F)n3)C2C)CC1]

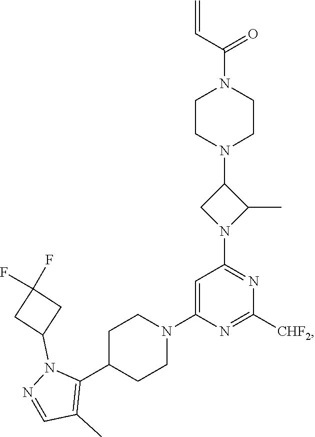 [SMILES: C=CC(=O)N1CCN(C2CN(c3cc(N4CCC(c5c(C)cnn5C5CC(F)(F)C5)CC4)nc(C(F)F)n3)C2C)CC1]

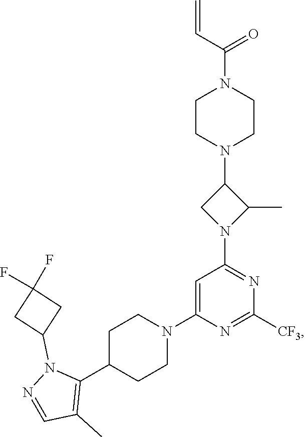 [SMILES: C=CC(=O)N1CCN(C2CN(c3cc(N4CCC(c5c(C)cnn5C5CC(F)(F)C5)CC4)nc(C(F)(F)F)n3)C2C)CC1]

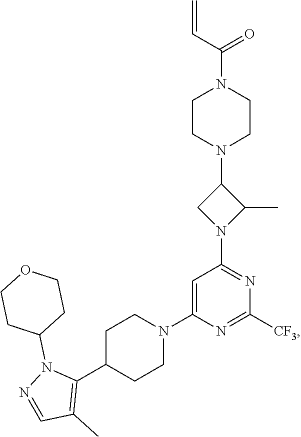 [SMILES: C=CC(=O)N1CCN(C2CN(c3cc(N4CCC(c5c(C)cnn5C5CCOCC5)CC4)nc(C(F)(F)F)n3)C2C)CC1]

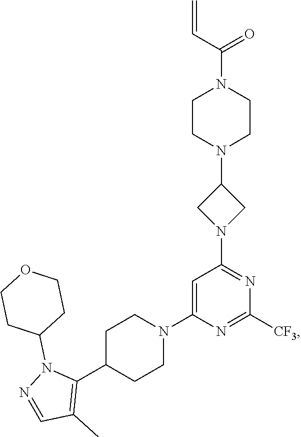 [SMILES: C=CC(=O)N1CCN(C2CN(c3cc(N4CCC(c5c(C)cnn5C5CCOCC5)CC4)nc(C(F)(F)F)n3)C2)CC1]

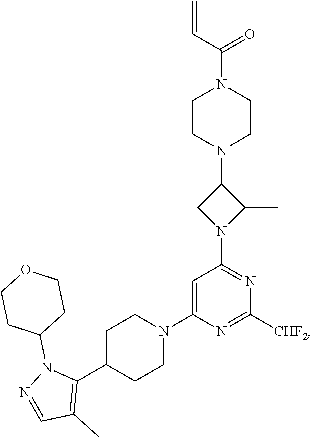 [SMILES: C=CC(=O)N1CCN(C2CN(c3cc(N4CCC(c5c(C)cnn5C5CCOCC5)CC4)nc(C(F)F)n3)C2C)CC1]

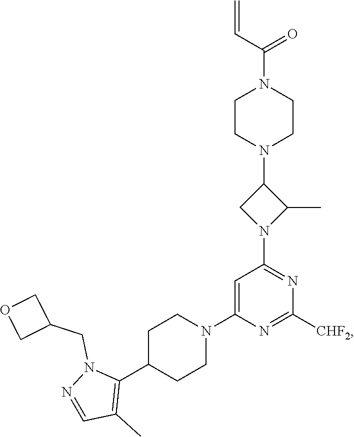 [SMILES: C=CC(=O)N1CCN(C2CN(c3cc(N4CCC(c5c(C)cnn5CC5COC5)CC4)nc(C(F)F)n3)C2C)CC1]

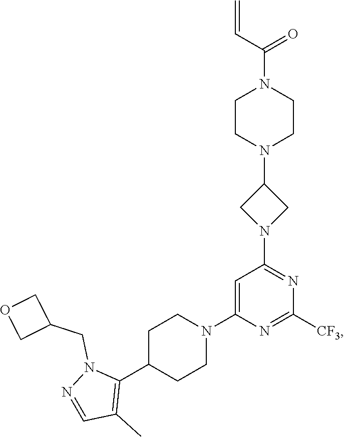 [SMILES: C=CC(=O)N1CCN(C2CN(c3cc(N4CCC(c5c(C)cnn5CC5COC5)CC4)nc(C(F)(F)F)n3)C2)CC1]

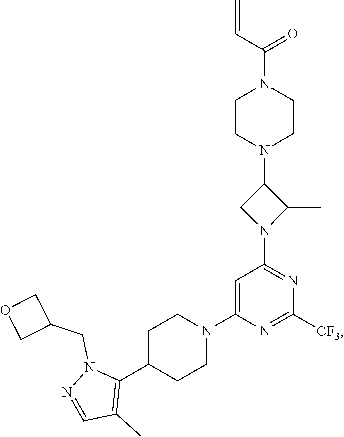 [SMILES: C=CC(=O)N1CCN(C2CN(c3cc(N4CCC(c5c(C)cnn5CC5COC5)CC4)nc(C(F)(F)F)n3)C2C)CC1]

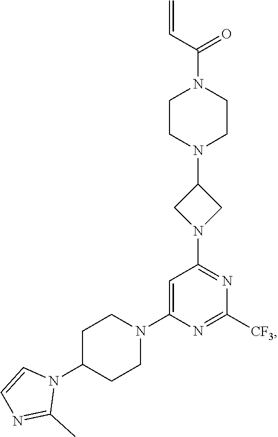 [SMILES: C=CC(=O)N1CCN(C2CN(c3cc(N4CCC(n5ccnc5C)CC4)nc(C(F)(F)F)n3)C2)CC1]

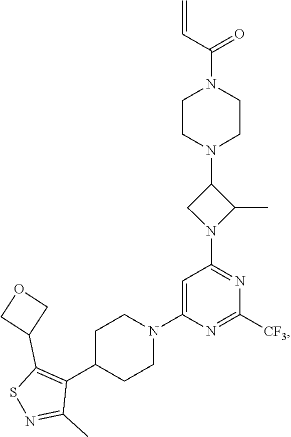 [SMILES: C=CC(=O)N1CCN(C2CN(c3cc(N4CCC(c5c(C)nsc5C5COC5)CC4)nc(C(F)(F)F)n3)C2C)CC1]

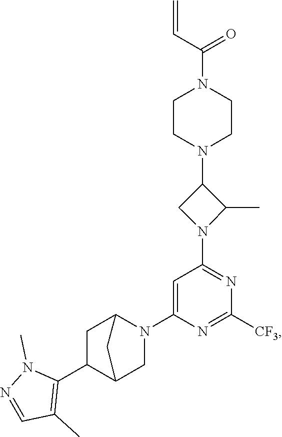 [SMILES: C=CC(=O)N1CCN(C2CN(c3cc(N4CC5CC4CC5c4c(C)cnn4C)nc(C(F)(F)F)n3)C2C)CC1]

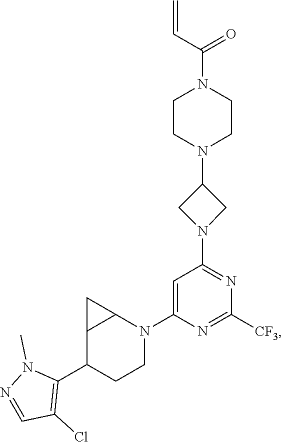 [SMILES: C=CC(=O)N1CCN(C2CN(c3cc(N4CCC(c5c(Cl)cnn5C)C5CC54)nc(C(F)(F)F)n3)C2)CC1]

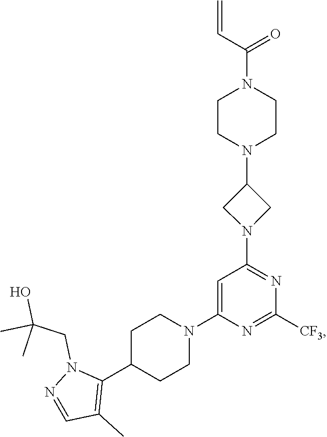 [SMILES: C=CC(=O)N1CCN(C2CN(c3cc(N4CCC(c5c(C)cnn5CC(C)(C)O)CC4)nc(C(F)(F)F)n3)C2)CC1]

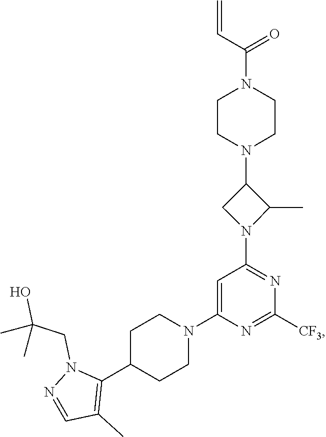 [SMILES: C=CC(=O)N1CCN(C2CN(c3cc(N4CCC(c5c(C)cnn5CC(C)(C)O)CC4)nc(C(F)(F)F)n3)C2C)CC1]

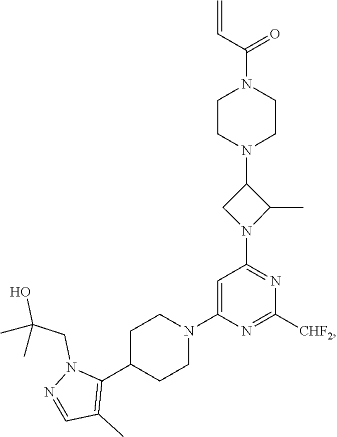 [SMILES: C=CC(=O)N1CCN(C2CN(c3cc(N4CCC(c5c(C)cnn5CC(C)(C)O)CC4)nc(C(F)F)n3)C2C)CC1]

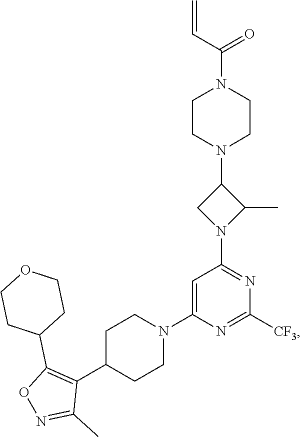 [SMILES: C=CC(=O)N1CCN(C2CN(c3cc(N4CCC(c5c(C)noc5C5CCOCC5)CC4)nc(C(F)(F)F)n3)C2C)CC1]

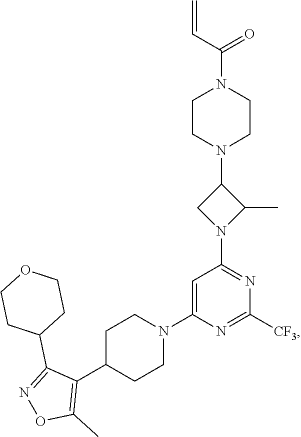 [SMILES: C=CC(=O)N1CCN(C2CN(c3cc(N4CCC(c5c(C6CCOCC6)noc5C)CC4)nc(C(F)(F)F)n3)C2C)CC1]

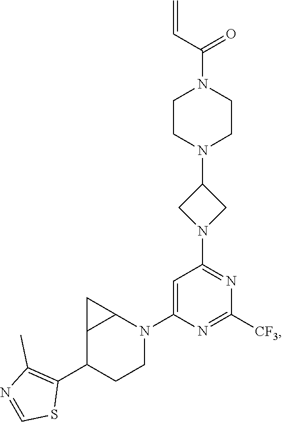 [SMILES: C=CC(=O)N1CCN(C2CN(c3cc(N4CCC(c5scnc5C)C5CC54)nc(C(F)(F)F)n3)C2)CC1]

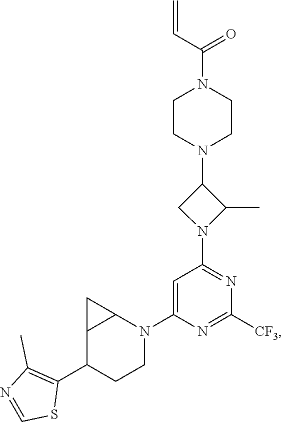 [SMILES: C=CC(=O)N1CCN(C2CN(c3cc(N4CCC(c5scnc5C)C5CC54)nc(C(F)(F)F)n3)C2C)CC1]

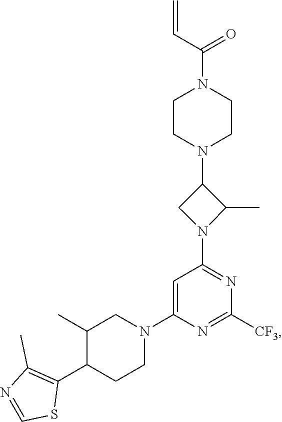 [SMILES: C=CC(=O)N1CCN(C2CN(c3cc(N4CCC(c5scnc5C)C(C)C4)nc(C(F)(F)F)n3)C2C)CC1]

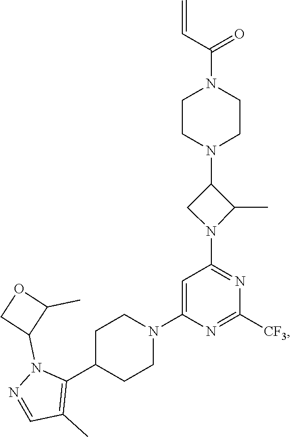 [SMILES: C=CC(=O)N1CCN(C2CN(c3cc(N4CCC(c5c(C)cnn5C5COC5C)CC4)nc(C(F)(F)F)n3)C2C)CC1]

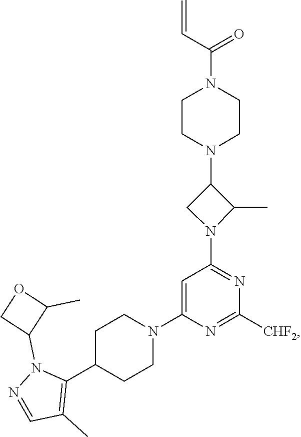 [SMILES: C=CC(=O)N1CCN(C2CN(c3cc(N4CCC(c5c(C)cnn5C5COC5C)CC4)nc(C(F)F)n3)C2C)CC1]

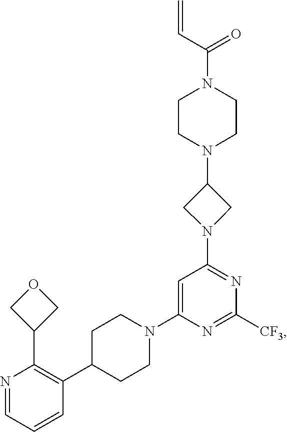 [SMILES: C=CC(=O)N1CCN(C2CN(c3cc(N4CCC(c5cccnc5C5COC5)CC4)nc(C(F)(F)F)n3)C2)CC1]

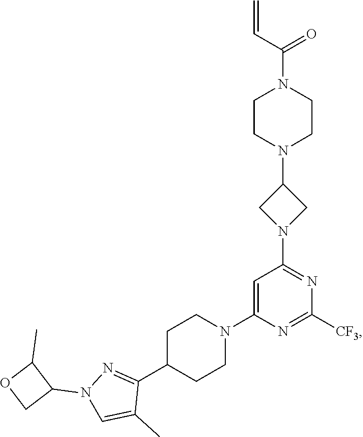 [SMILES: C=CC(=O)N1CCN(C2CN(c3cc(N4CCC(c5nn(C6COC6C)cc5C)CC4)nc(C(F)(F)F)n3)C2)CC1]

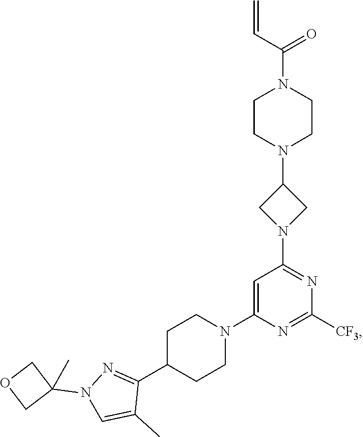 [SMILES: C=CC(=O)N1CCN(C2CN(c3cc(N4CCC(c5nn(C6(C)COC6)cc5C)CC4)nc(C(F)(F)F)n3)C2)CC1]

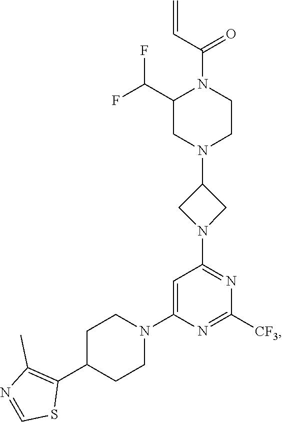 [SMILES: C=CC(=O)N1CCN(C2CN(c3cc(N4CCC(c5scnc5C)CC4)nc(C(F)(F)F)n3)C2)CC1C(F)F]

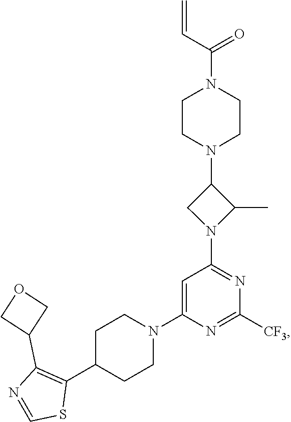 [SMILES: C=CC(=O)N1CCN(C2CN(c3cc(N4CCC(c5scnc5C5COC5)CC4)nc(C(F)(F)F)n3)C2C)CC1]

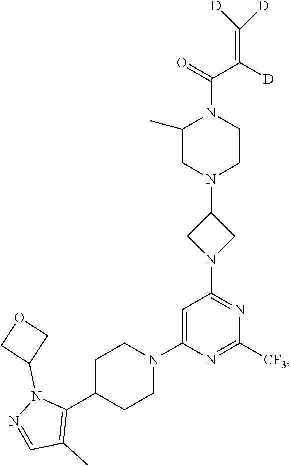 [SMILES: [2H]C([2H])=C([2H])C(=O)N1CCN(C2CN(c3cc(N4CCC(c5c(C)cnn5C5COC5)CC4)nc(C(F)(F)F)n3)C2)CC1C]

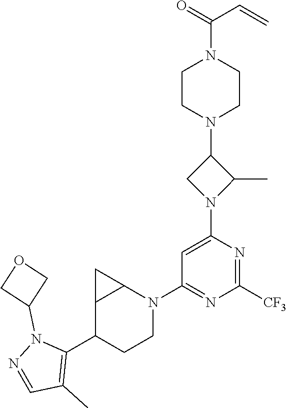 [SMILES: C=CC(=O)N1CCN(C2CN(c3cc(N4CCC(c5c(C)cnn5C5COC5)C5CC54)nc(C(F)(F)F)n3)C2C)CC1]

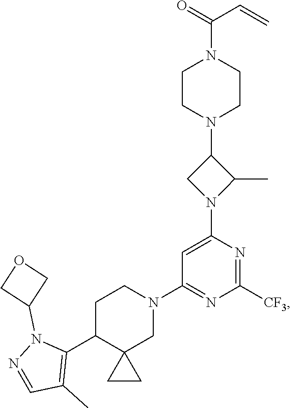 [SMILES: C=CC(=O)N1CCN(C2CN(c3cc(N4CCC(c5c(C)cnn5C5COC5)C5(CC5)C4)nc(C(F)(F)F)n3)C2C)CC1]

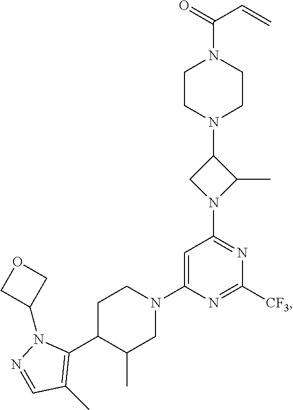 [SMILES: C=CC(=O)N1CCN(C2CN(c3cc(N4CCC(c5c(C)cnn5C5COC5)C(C)C4)nc(C(F)(F)F)n3)C2C)CC1]

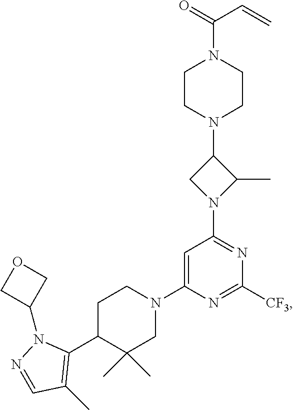 [SMILES: C=CC(=O)N1CCN(C2CN(c3cc(N4CCC(c5c(C)cnn5C5COC5)C(C)(C)C4)nc(C(F)(F)F)n3)C2C)CC1]

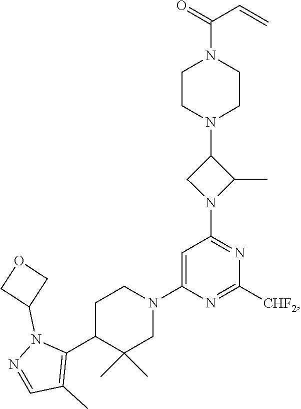 [SMILES: C=CC(=O)N1CCN(C2CN(c3cc(N4CCC(c5c(C)cnn5C5COC5)C(C)(C)C4)nc(C(F)F)n3)C2C)CC1]

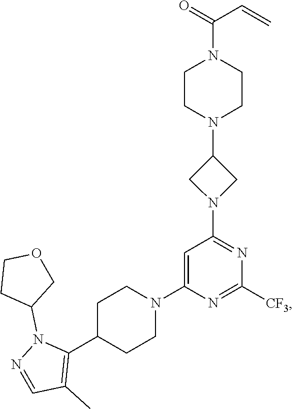 [SMILES: C=CC(=O)N1CCN(C2CN(c3cc(N4CCC(c5c(C)cnn5C5CCOC5)CC4)nc(C(F)(F)F)n3)C2)CC1]

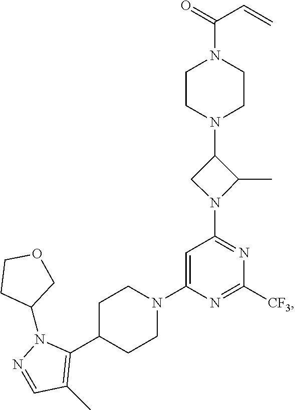 [SMILES: C=CC(=O)N1CCN(C2CN(c3cc(N4CCC(c5c(C)cnn5C5CCOC5)CC4)nc(C(F)(F)F)n3)C2C)CC1]

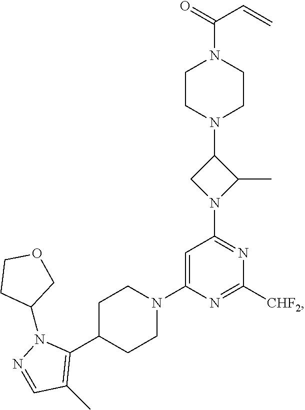 [SMILES: C=CC(=O)N1CCN(C2CN(c3cc(N4CCC(c5c(C)cnn5C5CCOC5)CC4)nc(C(F)F)n3)C2C)CC1]

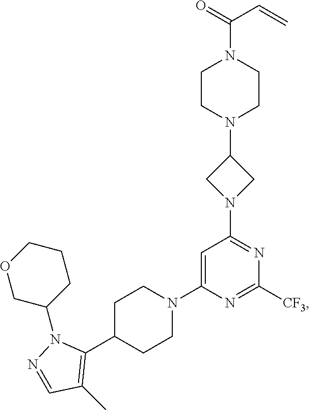 [SMILES: C=CC(=O)N1CCN(C2CN(c3cc(N4CCC(c5c(C)cnn5C5CCCOC5)CC4)nc(C(F)(F)F)n3)C2)CC1]

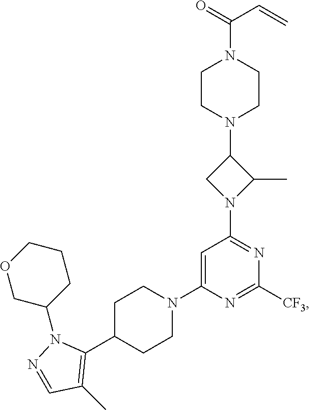 [SMILES: C=CC(=O)N1CCN(C2CN(c3cc(N4CCC(c5c(C)cnn5C5CCCOC5)CC4)nc(C(F)(F)F)n3)C2C)CC1]

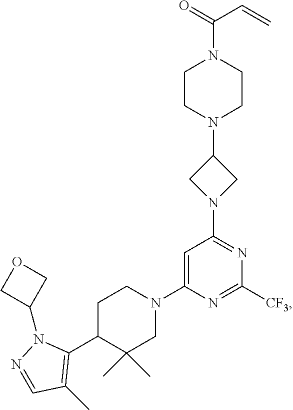 [SMILES: C=CC(=O)N1CCN(C2CN(c3cc(N4CCC(c5c(C)cnn5C5COC5)C(C)(C)C4)nc(C(F)(F)F)n3)C2)CC1]

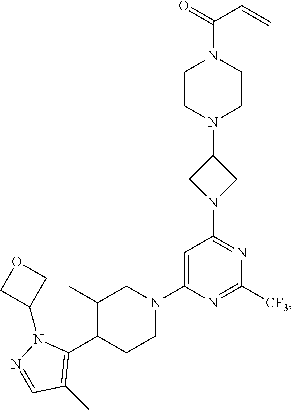 [SMILES: C=CC(=O)N1CCN(C2CN(c3cc(N4CCC(c5c(C)cnn5C5COC5)C(C)C4)nc(C(F)(F)F)n3)C2)CC1]

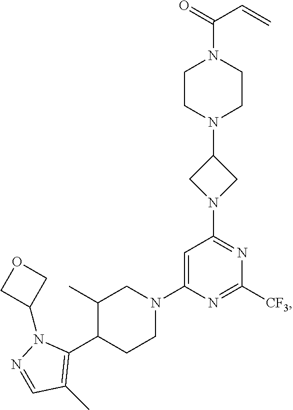 [SMILES: C=CC(=O)N1CCN(C2CN(c3cc(N4CCC(c5c(C)cnn5C5COC5)C(C)C4)nc(C(F)(F)F)n3)C2)CC1]

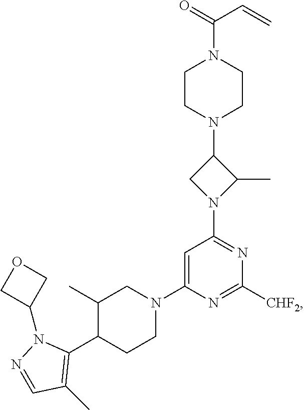 [SMILES: C=CC(=O)N1CCN(C2CN(c3cc(N4CCC(c5c(C)cnn5C5COC5)C(C)C4)nc(C(F)F)n3)C2C)CC1]

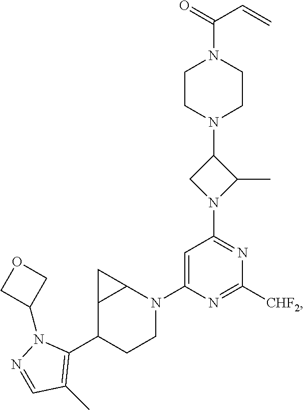 [SMILES: C=CC(=O)N1CCN(C2CN(c3cc(N4CCC(c5c(C)cnn5C5COC5)C5CC54)nc(C(F)F)n3)C2C)CC1]

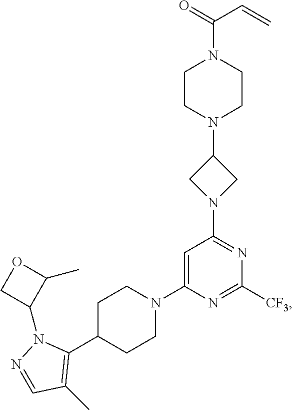 [SMILES: C=CC(=O)N1CCN(C2CN(c3cc(N4CCC(c5c(C)cnn5C5COC5C)CC4)nc(C(F)(F)F)n3)C2)CC1]

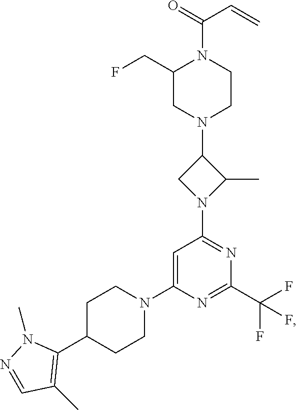 [SMILES: C=CC(=O)N1CCN(C2CN(c3cc(N4CCC(c5c(C)cnn5C)CC4)nc(C(F)(F)F)n3)C2C)CC1CF]

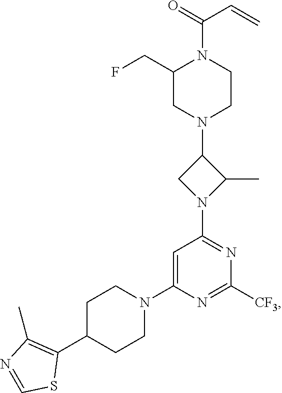 [SMILES: C=CC(=O)N1CCN(C2CN(c3cc(N4CCC(c5scnc5C)CC4)nc(C(F)(F)F)n3)C2C)CC1CF]

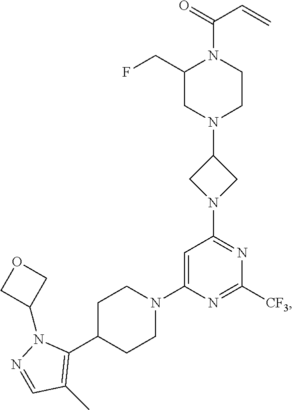 [SMILES: C=CC(=O)N1CCN(C2CN(c3cc(N4CCC(c5c(C)cnn5C5COC5)CC4)nc(C(F)(F)F)n3)C2)CC1CF]

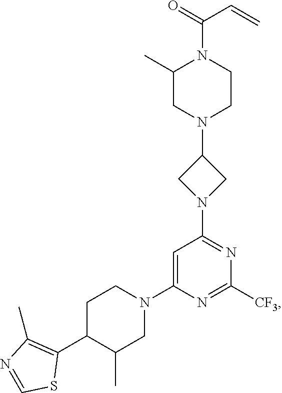 [SMILES: C=CC(=O)N1CCN(C2CN(c3cc(N4CCC(c5scnc5C)C(C)C4)nc(C(F)(F)F)n3)C2)CC1C]

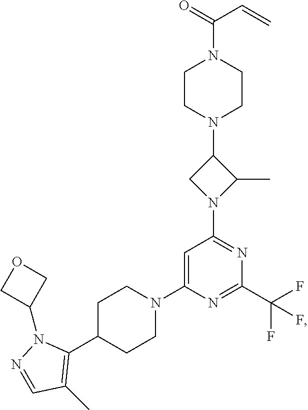 [SMILES: C=CC(=O)N1CCN(C2CN(c3cc(N4CCC(c5c(C)cnn5C5COC5)CC4)nc(C(F)(F)F)n3)C2C)CC1]

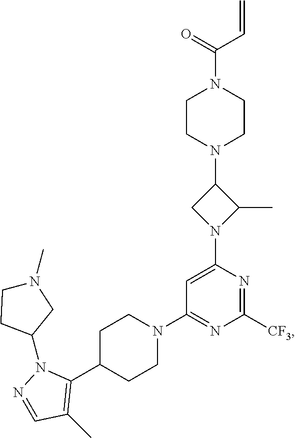 [SMILES: C=CC(=O)N1CCN(C2CN(c3cc(N4CCC(c5c(C)cnn5C5CCN(C)C5)CC4)nc(C(F)(F)F)n3)C2C)CC1]

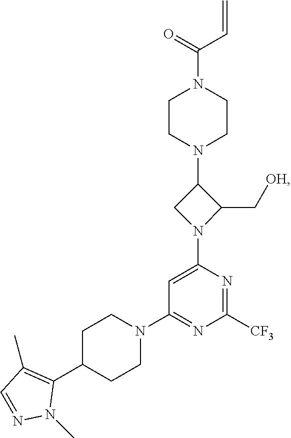 [SMILES: C=CC(=O)N1CCN(C2CN(c3cc(N4CCC(c5c(C)cnn5C)CC4)nc(C(F)(F)F)n3)C2CO)CC1]

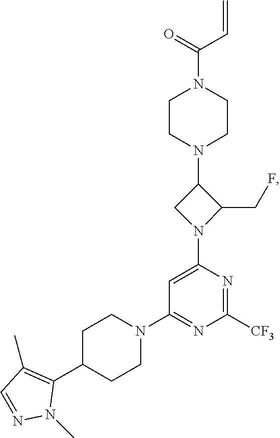 [SMILES: C=CC(=O)N1CCN(C2CN(c3cc(N4CCC(c5c(C)cnn5C)CC4)nc(C(F)(F)F)n3)C2CF)CC1]

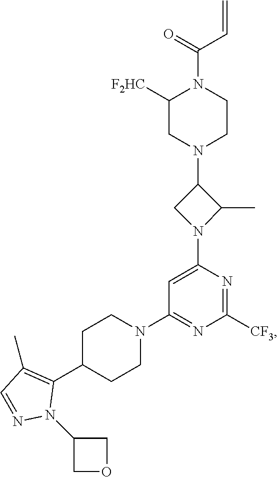 [SMILES: C=CC(=O)N1CCN(C2CN(c3cc(N4CCC(c5c(C)cnn5C5COC5)CC4)nc(C(F)(F)F)n3)C2C)CC1C(F)F]

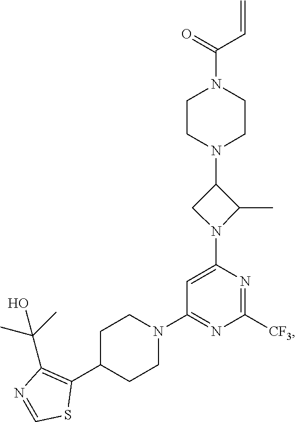 [SMILES: C=CC(=O)N1CCN(C2CN(c3cc(N4CCC(c5scnc5C(C)(C)O)CC4)nc(C(F)(F)F)n3)C2C)CC1]

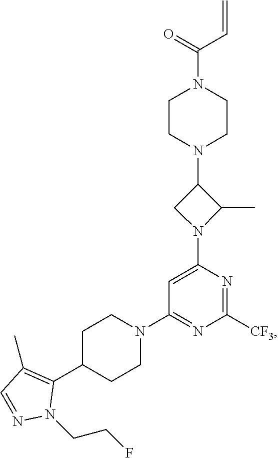 [SMILES: C=CC(=O)N1CCN(C2CN(c3cc(N4CCC(c5c(C)cnn5CCF)CC4)nc(C(F)(F)F)n3)C2C)CC1]

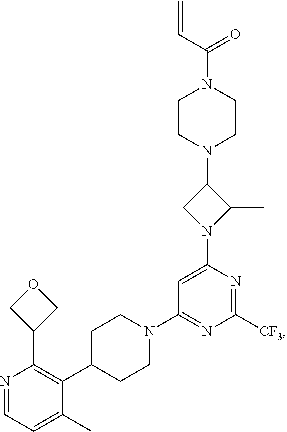 [SMILES: C=CC(=O)N1CCN(C2CN(c3cc(N4CCC(c5c(C)ccnc5C5COC5)CC4)nc(C(F)(F)F)n3)C2C)CC1]

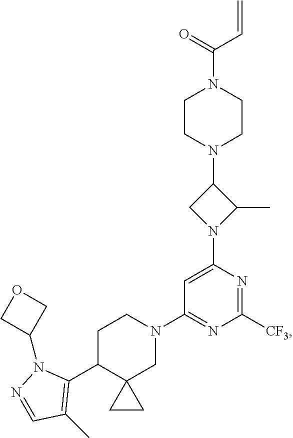 [SMILES: C=CC(=O)N1CCN(C2CN(c3cc(N4CCC(c5c(C)cnn5C5COC5)C5(CC5)C4)nc(C(F)(F)F)n3)C2C)CC1]

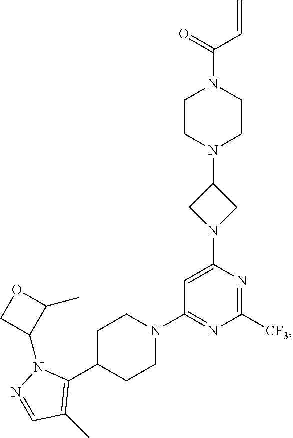 [SMILES: C=CC(=O)N1CCN(C2CN(c3cc(N4CCC(c5c(C)cnn5C5COC5C)CC4)nc(C(F)(F)F)n3)C2)CC1]

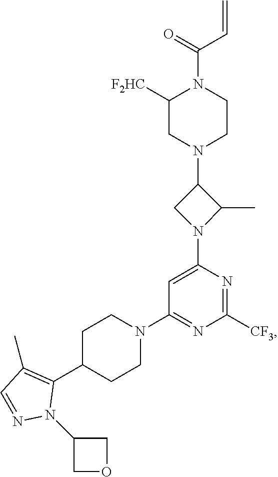 [SMILES: C=CC(=O)N1CCN(C2CN(c3cc(N4CCC(c5c(C)cnn5C5COC5)CC4)nc(C(F)(F)F)n3)C2C)CC1C(F)F]

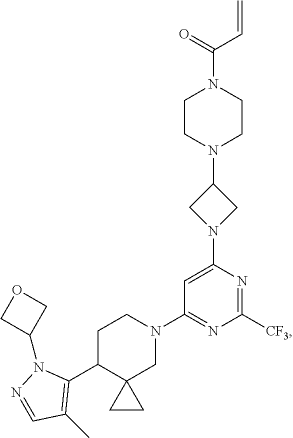 [SMILES: C=CC(=O)N1CCN(C2CN(c3cc(N4CCC(c5c(C)cnn5C5COC5)C5(CC5)C4)nc(C(F)(F)F)n3)C2)CC1]

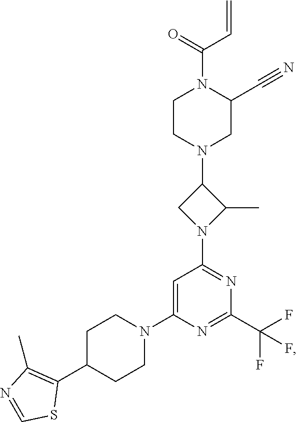 [SMILES: C=CC(=O)N1CCN(C2CN(c3cc(N4CCC(c5scnc5C)CC4)nc(C(F)(F)F)n3)C2C)CC1C#N]

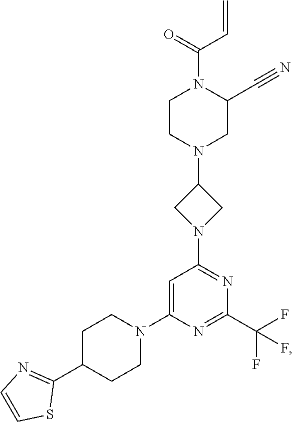 [SMILES: C=CC(=O)N1CCN(C2CN(c3cc(N4CCC(c5nccs5)CC4)nc(C(F)(F)F)n3)C2)CC1C#N]

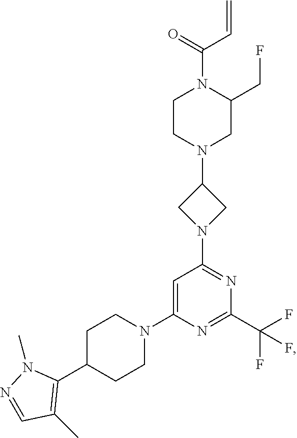 [SMILES: C=CC(=O)N1CCN(C2CN(c3cc(N4CCC(c5c(C)cnn5C)CC4)nc(C(F)(F)F)n3)C2)CC1CF]

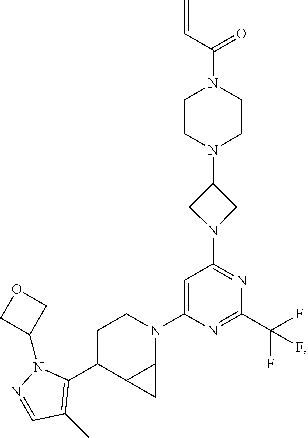 [SMILES: C=CC(=O)N1CCN(C2CN(c3cc(N4CCC(c5c(C)cnn5C5COC5)C5CC54)nc(C(F)(F)F)n3)C2)CC1]

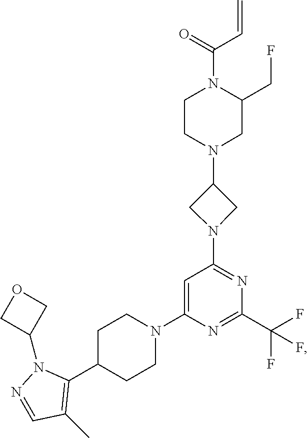 [SMILES: C=CC(=O)N1CCN(C2CN(c3cc(N4CCC(c5c(C)cnn5C5COC5)CC4)nc(C(F)(F)F)n3)C2)CC1CF]

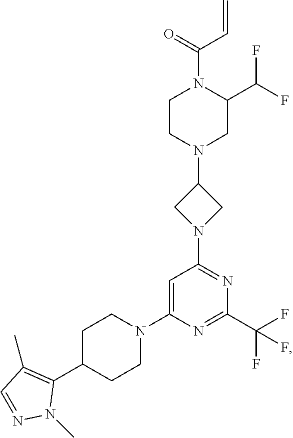 [SMILES: C=CC(=O)N1CCN(C2CN(c3cc(N4CCC(c5c(C)cnn5C)CC4)nc(C(F)(F)F)n3)C2)CC1C(F)F]

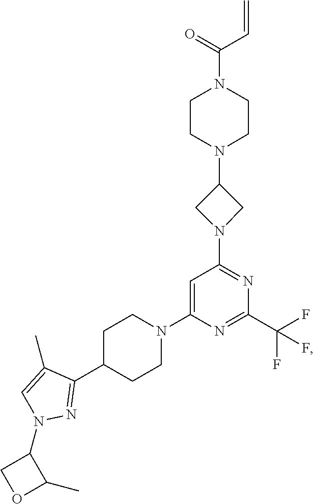 [SMILES: C=CC(=O)N1CCN(C2CN(c3cc(N4CCC(c5nn(C6COC6C)cc5C)CC4)nc(C(F)(F)F)n3)C2)CC1]

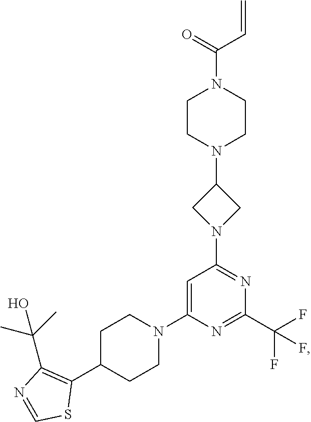 [SMILES: C=CC(=O)N1CCN(C2CN(c3cc(N4CCC(c5scnc5C(C)(C)O)CC4)nc(C(F)(F)F)n3)C2)CC1]

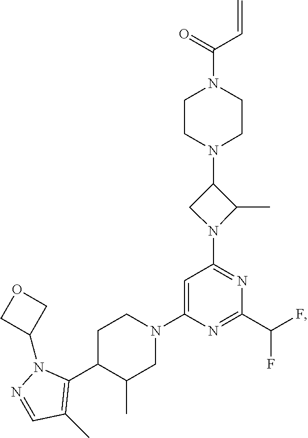 [SMILES: C=CC(=O)N1CCN(C2CN(c3cc(N4CCC(c5c(C)cnn5C5COC5)C(C)C4)nc(C(F)F)n3)C2C)CC1]

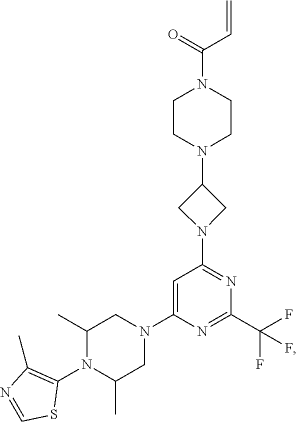 [SMILES: C=CC(=O)N1CCN(C2CN(c3cc(N4CC(C)N(c5scnc5C)C(C)C4)nc(C(F)(F)F)n3)C2)CC1]

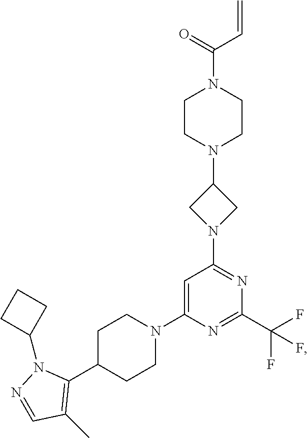 [SMILES: C=CC(=O)N1CCN(C2CN(c3cc(N4CCC(c5c(C)cnn5C5CCC5)CC4)nc(C(F)(F)F)n3)C2)CC1]

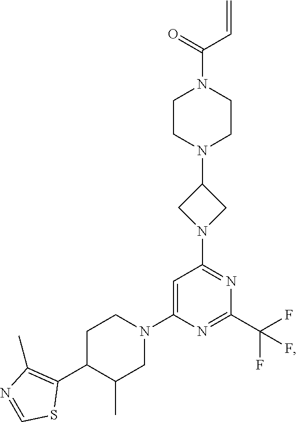 [SMILES: C=CC(=O)N1CCN(C2CN(c3cc(N4CCC(c5scnc5C)C(C)C4)nc(C(F)(F)F)n3)C2)CC1]

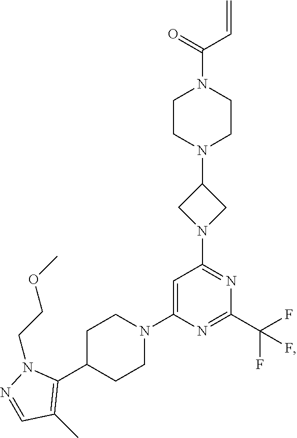 [SMILES: C=CC(=O)N1CCN(C2CN(c3cc(N4CCC(c5c(C)cnn5CCOC)CC4)nc(C(F)(F)F)n3)C2)CC1]

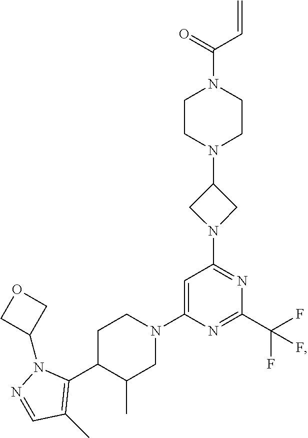 [SMILES: C=CC(=O)N1CCN(C2CN(c3cc(N4CCC(c5c(C)cnn5C5COC5)C(C)C4)nc(C(F)(F)F)n3)C2)CC1]

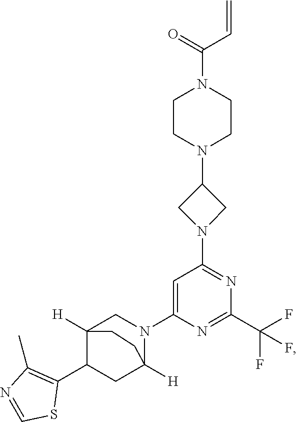 [SMILES: C=CC(=O)N1CCN(C2CN(c3cc(N4CC5CCC4CC5c4scnc4C)nc(C(F)(F)F)n3)C2)CC1]

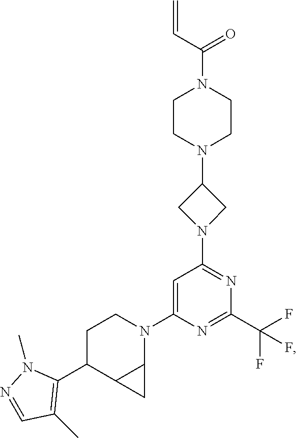 [SMILES: C=CC(=O)N1CCN(C2CN(c3cc(N4CCC(c5c(C)cnn5C)C5CC54)nc(C(F)(F)F)n3)C2)CC1]

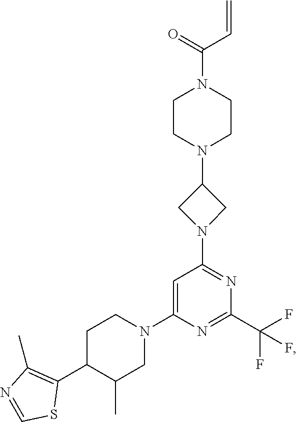 [SMILES: C=CC(=O)N1CCN(C2CN(c3cc(N4CCC(c5scnc5C)C(C)C4)nc(C(F)(F)F)n3)C2)CC1]

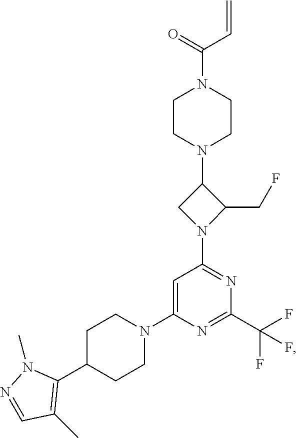 [SMILES: C=CC(=O)N1CCN(C2CN(c3cc(N4CCC(c5c(C)cnn5C)CC4)nc(C(F)(F)F)n3)C2CF)CC1]

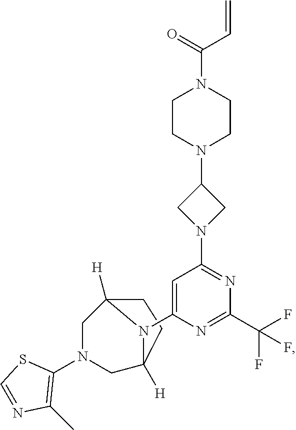 [SMILES: C=CC(=O)N1CCN(C2CN(c3cc(N4C5CCC4CN(c4scnc4C)C5)nc(C(F)(F)F)n3)C2)CC1]

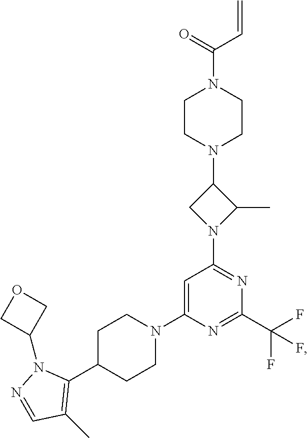 [SMILES: C=CC(=O)N1CCN(C2CN(c3cc(N4CCC(c5c(C)cnn5C5COC5)CC4)nc(C(F)(F)F)n3)C2C)CC1]

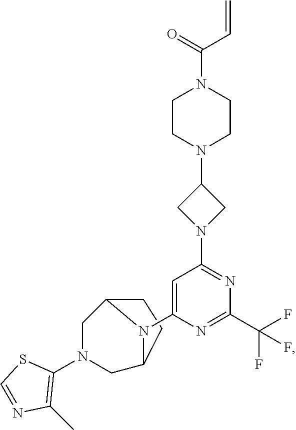 [SMILES: C=CC(=O)N1CCN(C2CN(c3cc(N4C5CCC4CN(c4scnc4C)C5)nc(C(F)(F)F)n3)C2)CC1]

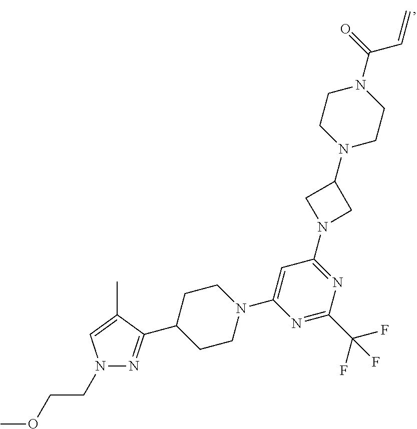 [SMILES: C=CC(=O)N1CCN(C2CN(c3cc(N4CCC(c5nn(CCOC)cc5C)CC4)nc(C(F)(F)F)n3)C2)CC1]

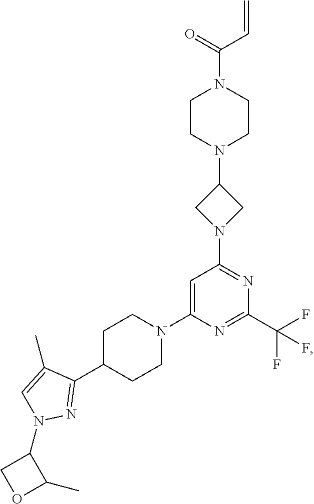 [SMILES: C=CC(=O)N1CCN(C2CN(c3cc(N4CCC(c5nn(C6COC6C)cc5C)CC4)nc(C(F)(F)F)n3)C2)CC1]

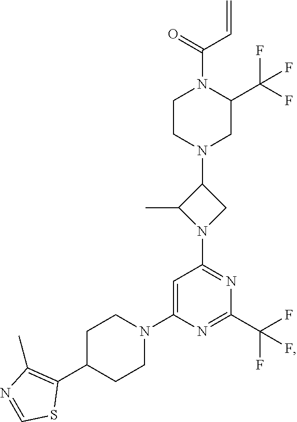 [SMILES: C=CC(=O)N1CCN(C2CN(c3cc(N4CCC(c5scnc5C)CC4)nc(C(F)(F)F)n3)C2C)CC1C(F)(F)F]

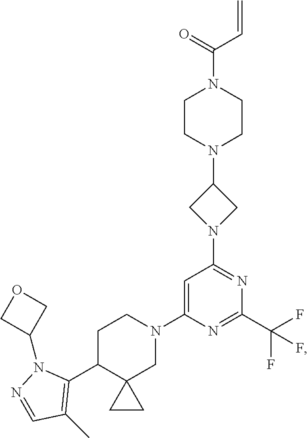 [SMILES: C=CC(=O)N1CCN(C2CN(c3cc(N4CCC(c5c(C)cnn5C5COC5)C5(CC5)C4)nc(C(F)(F)F)n3)C2)CC1]

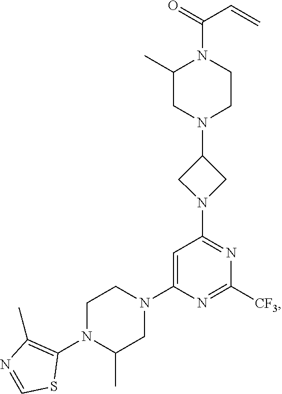 [SMILES: C=CC(=O)N1CCN(C2CN(c3cc(N4CCN(c5scnc5C)C(C)C4)nc(C(F)(F)F)n3)C2)CC1C]

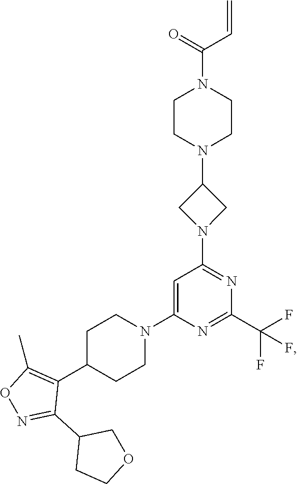 [SMILES: C=CC(=O)N1CCN(C2CN(c3cc(N4CCC(c5c(C6CCOC6)noc5C)CC4)nc(C(F)(F)F)n3)C2)CC1]

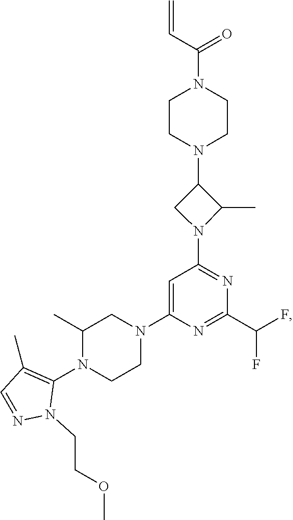 [SMILES: C=CC(=O)N1CCN(C2CN(c3cc(N4CCN(c5c(C)cnn5CCOC)C(C)C4)nc(C(F)F)n3)C2C)CC1]

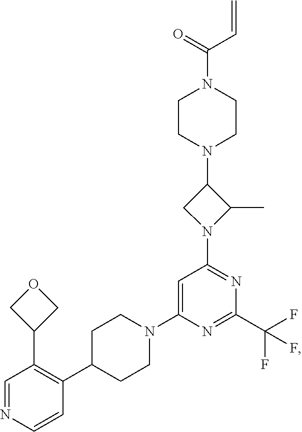 [SMILES: C=CC(=O)N1CCN(C2CN(c3cc(N4CCC(c5ccncc5C5COC5)CC4)nc(C(F)(F)F)n3)C2C)CC1]

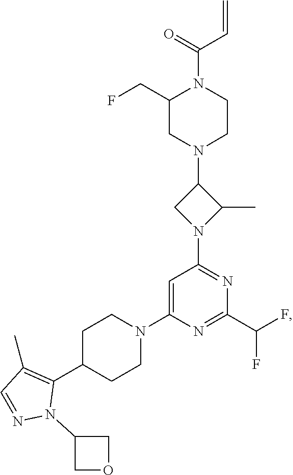 [SMILES: C=CC(=O)N1CCN(C2CN(c3cc(N4CCC(c5c(C)cnn5C5COC5)CC4)nc(C(F)F)n3)C2C)CC1CF]

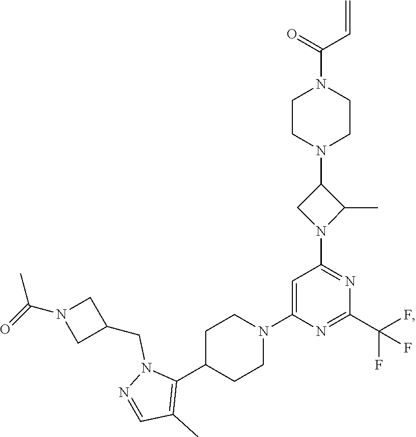 [SMILES: C=CC(=O)N1CCN(C2CN(c3cc(N4CCC(c5c(C)cnn5CC5CN(C(C)=O)C5)CC4)nc(C(F)(F)F)n3)C2C)CC1]

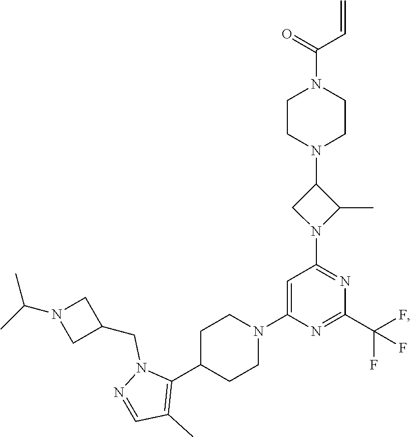 [SMILES: C=CC(=O)N1CCN(C2CN(c3cc(N4CCC(c5c(C)cnn5CC5CN(C(C)C)C5)CC4)nc(C(F)(F)F)n3)C2C)CC1]

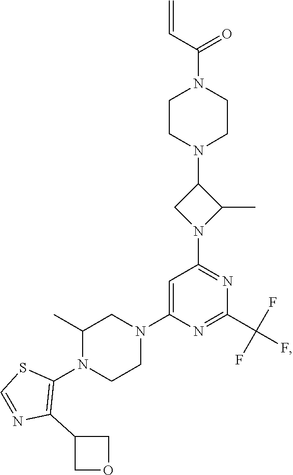 [SMILES: C=CC(=O)N1CCN(C2CN(c3cc(N4CCN(c5scnc5C5COC5)C(C)C4)nc(C(F)(F)F)n3)C2C)CC1]

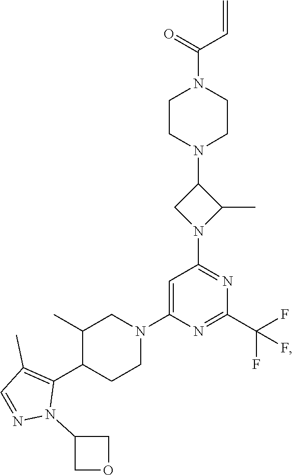 [SMILES: C=CC(=O)N1CCN(C2CN(c3cc(N4CCC(c5c(C)cnn5C5COC5)C(C)C4)nc(C(F)(F)F)n3)C2C)CC1]

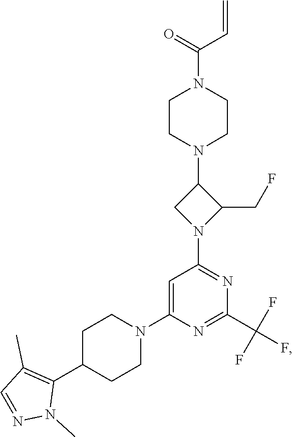 [SMILES: C=CC(=O)N1CCN(C2CN(c3cc(N4CCC(c5c(C)cnn5C)CC4)nc(C(F)(F)F)n3)C2CF)CC1]

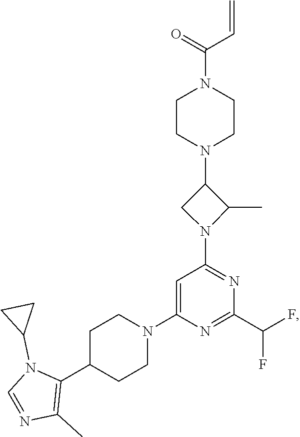 [SMILES: C=CC(=O)N1CCN(C2CN(c3cc(N4CCC(c5c(C)ncn5C5CC5)CC4)nc(C(F)F)n3)C2C)CC1]

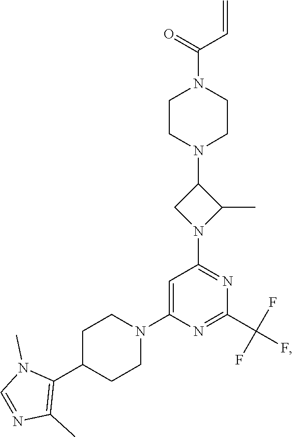 [SMILES: C=CC(=O)N1CCN(C2CN(c3cc(N4CCC(c5c(C)ncn5C)CC4)nc(C(F)(F)F)n3)C2C)CC1]

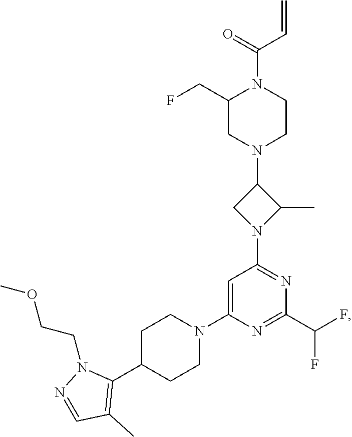 [SMILES: C=CC(=O)N1CCN(C2CN(c3cc(N4CCC(c5c(C)cnn5CCOC)CC4)nc(C(F)F)n3)C2C)CC1CF]

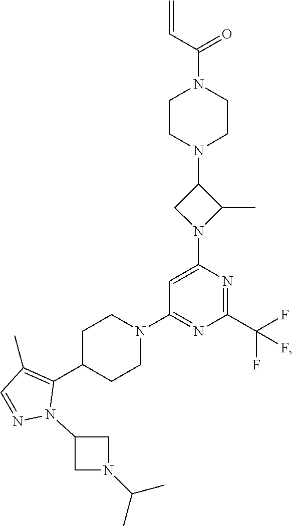 [SMILES: C=CC(=O)N1CCN(C2CN(c3cc(N4CCC(c5c(C)cnn5C5CN(C(C)C)C5)CC4)nc(C(F)(F)F)n3)C2C)CC1]

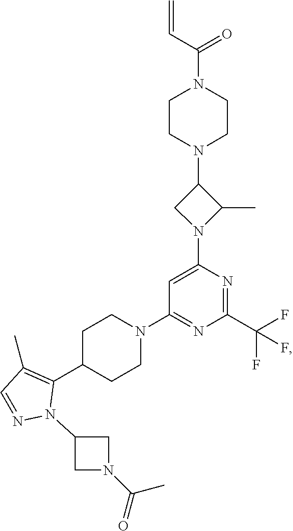 [SMILES: C=CC(=O)N1CCN(C2CN(c3cc(N4CCC(c5c(C)cnn5C5CN(C(C)=O)C5)CC4)nc(C(F)(F)F)n3)C2C)CC1]

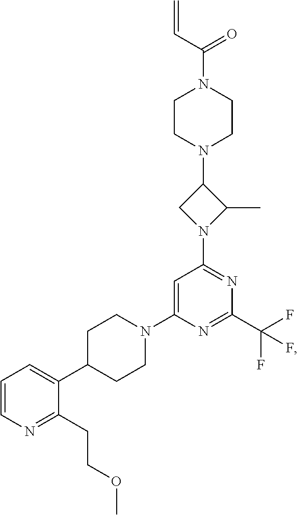 [SMILES: C=CC(=O)N1CCN(C2CN(c3cc(N4CCC(c5cccnc5CCOC)CC4)nc(C(F)(F)F)n3)C2C)CC1]

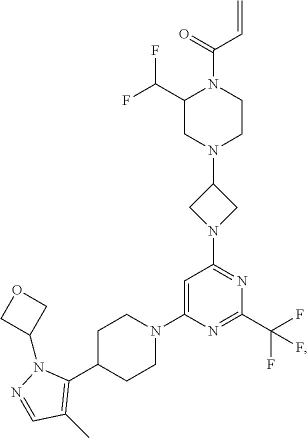 [SMILES: C=CC(=O)N1CCN(C2CN(c3cc(N4CCC(c5c(C)cnn5C5COC5)CC4)nc(C(F)(F)F)n3)C2)CC1C(F)F]

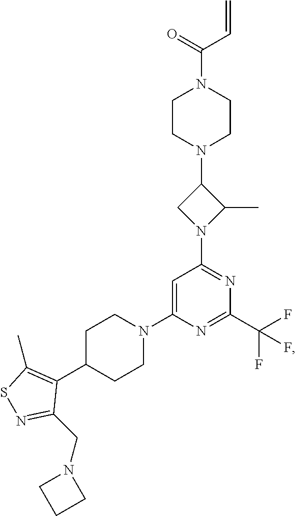 [SMILES: C=CC(=O)N1CCN(C2CN(c3cc(N4CCC(c5c(CN6CCC6)nsc5C)CC4)nc(C(F)(F)F)n3)C2C)CC1]

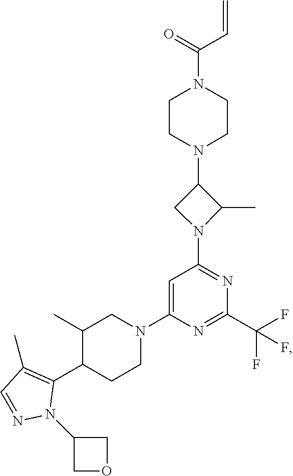 [SMILES: C=CC(=O)N1CCN(C2CN(c3cc(N4CCC(c5c(C)cnn5C5COC5)C(C)C4)nc(C(F)(F)F)n3)C2C)CC1]

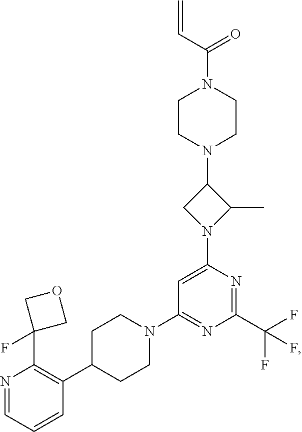 [SMILES: C=CC(=O)N1CCN(C2CN(c3cc(N4CCC(c5cccnc5C5(F)COC5)CC4)nc(C(F)(F)F)n3)C2C)CC1]

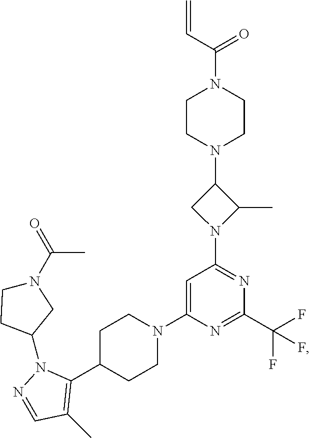 [SMILES: C=CC(=O)N1CCN(C2CN(c3cc(N4CCC(c5c(C)cnn5C5CCN(C(C)=O)C5)CC4)nc(C(F)(F)F)n3)C2C)CC1]

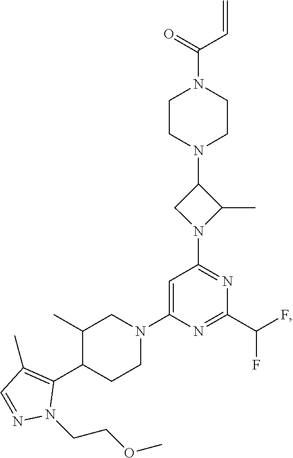 [SMILES: C=CC(=O)N1CCN(C2CN(c3cc(N4CCC(c5c(C)cnn5CCOC)C(C)C4)nc(C(F)F)n3)C2C)CC1]

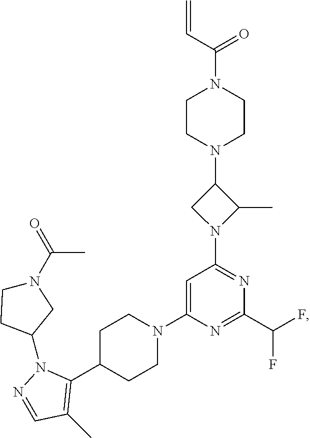 [SMILES: C=CC(=O)N1CCN(C2CN(c3cc(N4CCC(c5c(C)cnn5C5CCN(C(C)=O)C5)CC4)nc(C(F)F)n3)C2C)CC1]

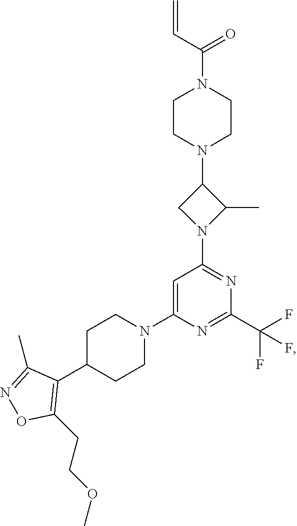 [SMILES: C=CC(=O)N1CCN(C2CN(c3cc(N4CCC(c5c(C)noc5CCOC)CC4)nc(C(F)(F)F)n3)C2C)CC1]

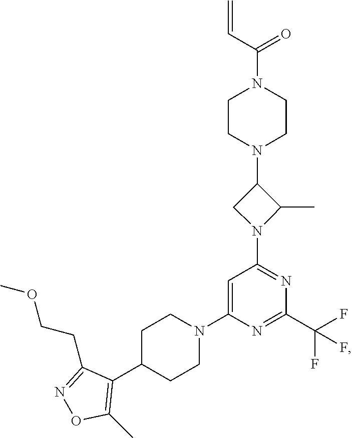 [SMILES: C=CC(=O)N1CCN(C2CN(c3cc(N4CCC(c5c(CCOC)noc5C)CC4)nc(C(F)(F)F)n3)C2C)CC1]

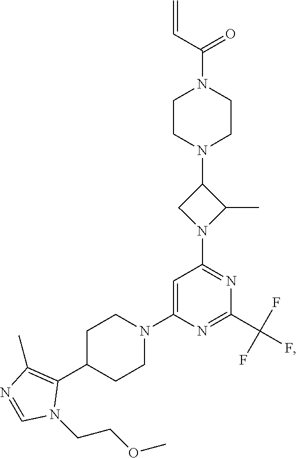 [SMILES: C=CC(=O)N1CCN(C2CN(c3cc(N4CCC(c5c(C)ncn5CCOC)CC4)nc(C(F)(F)F)n3)C2C)CC1]

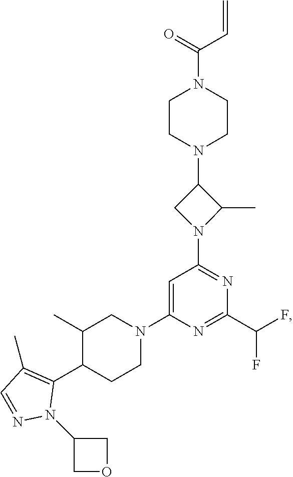 [SMILES: C=CC(=O)N1CCN(C2CN(c3cc(N4CCC(c5c(C)cnn5C5COC5)C(C)C4)nc(C(F)F)n3)C2C)CC1]

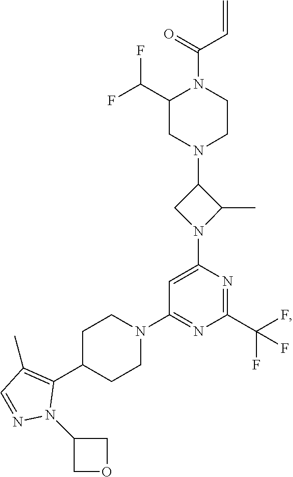 [SMILES: C=CC(=O)N1CCN(C2CN(c3cc(N4CCC(c5c(C)cnn5C5COC5)CC4)nc(C(F)(F)F)n3)C2C)CC1C(F)F]

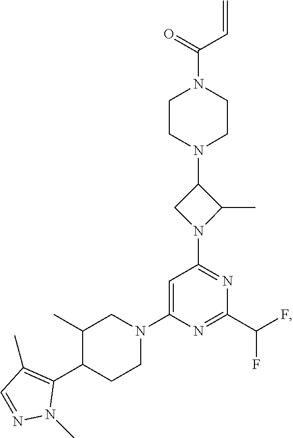 [SMILES: C=CC(=O)N1CCN(C2CN(c3cc(N4CCC(c5c(C)cnn5C)C(C)C4)nc(C(F)F)n3)C2C)CC1]

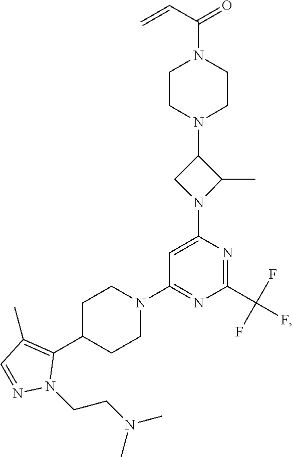 [SMILES: C=CC(=O)N1CCN(C2CN(c3cc(N4CCC(c5c(C)cnn5CCN(C)C)CC4)nc(C(F)(F)F)n3)C2C)CC1]

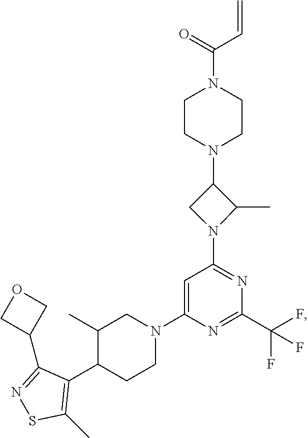 [SMILES: C=CC(=O)N1CCN(C2CN(c3cc(N4CCC(c5c(C6COC6)nsc5C)C(C)C4)nc(C(F)(F)F)n3)C2C)CC1]

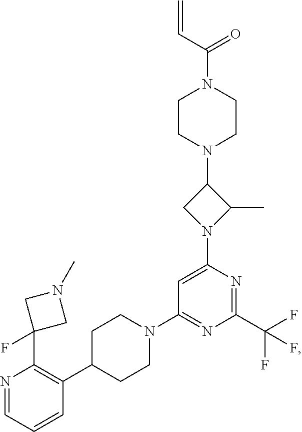 [SMILES: C=CC(=O)N1CCN(C2CN(c3cc(N4CCC(c5cccnc5C5(F)CN(C)C5)CC4)nc(C(F)(F)F)n3)C2C)CC1]

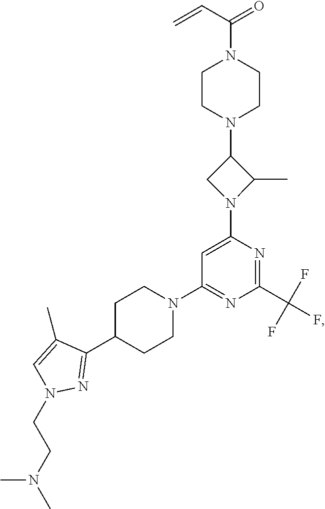 [SMILES: C=CC(=O)N1CCN(C2CN(c3cc(N4CCC(c5nn(CCN(C)C)cc5C)CC4)nc(C(F)(F)F)n3)C2C)CC1]

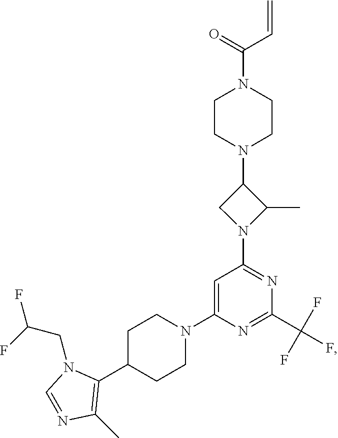 [SMILES: C=CC(=O)N1CCN(C2CN(c3cc(N4CCC(c5c(C)ncn5CC(F)F)CC4)nc(C(F)(F)F)n3)C2C)CC1]

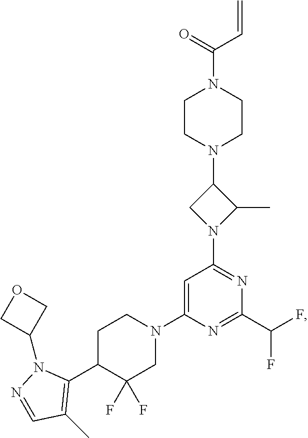 [SMILES: C=CC(=O)N1CCN(C2CN(c3cc(N4CCC(c5c(C)cnn5C5COC5)C(F)(F)C4)nc(C(F)F)n3)C2C)CC1]

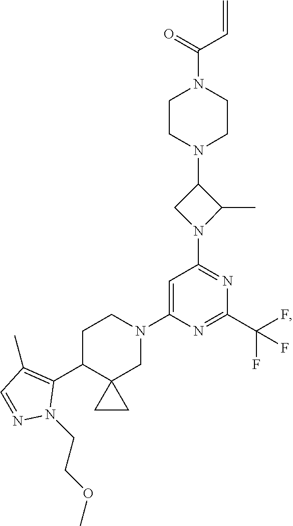 [SMILES: C=CC(=O)N1CCN(C2CN(c3cc(N4CCC(c5c(C)cnn5CCOC)C5(CC5)C4)nc(C(F)(F)F)n3)C2C)CC1]

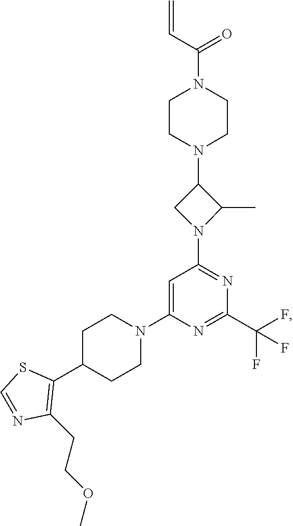 [SMILES: C=CC(=O)N1CCN(C2CN(c3cc(N4CCC(c5scnc5CCOC)CC4)nc(C(F)(F)F)n3)C2C)CC1]

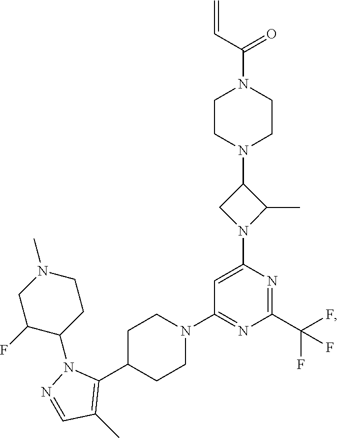 [SMILES: C=CC(=O)N1CCN(C2CN(c3cc(N4CCC(c5c(C)cnn5C5CCN(C)CC5F)CC4)nc(C(F)(F)F)n3)C2C)CC1]

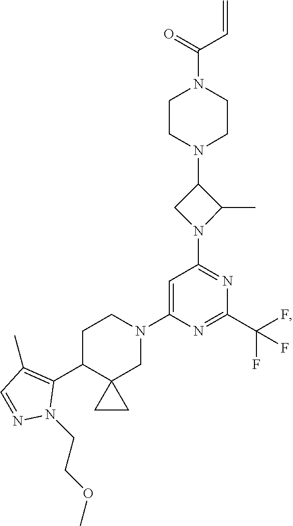 [SMILES: C=CC(=O)N1CCN(C2CN(c3cc(N4CCC(c5c(C)cnn5CCOC)C5(CC5)C4)nc(C(F)(F)F)n3)C2C)CC1]

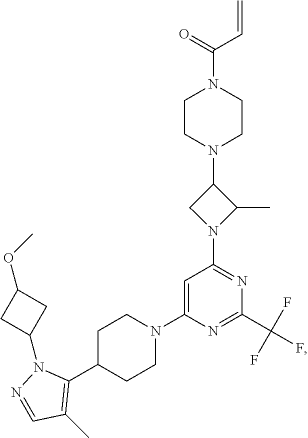 [SMILES: C=CC(=O)N1CCN(C2CN(c3cc(N4CCC(c5c(C)cnn5C5CC(OC)C5)CC4)nc(C(F)(F)F)n3)C2C)CC1]

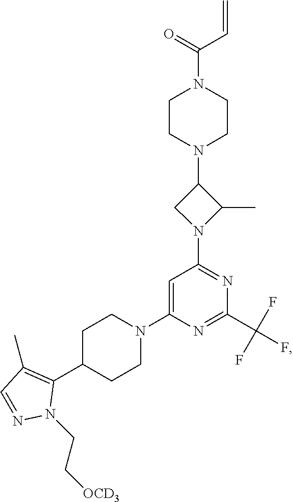

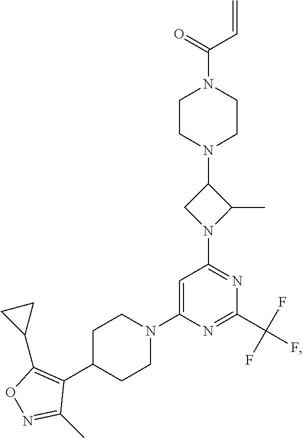 [SMILES: C=CC(=O)N1CCN(C2CN(c3cc(N4CCC(c5c(C)noc5C5CC5)CC4)nc(C(F)(F)F)n3)C2C)CC1]

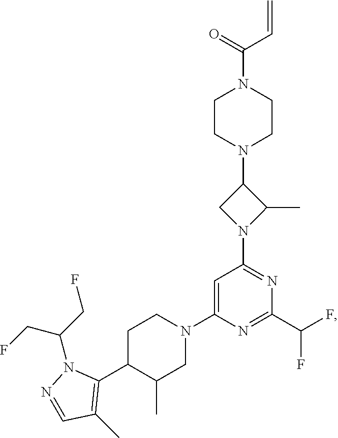 [SMILES: C=CC(=O)N1CCN(C2CN(c3cc(N4CCC(c5c(C)cnn5C(CF)CF)C(C)C4)nc(C(F)F)n3)C2C)CC1]

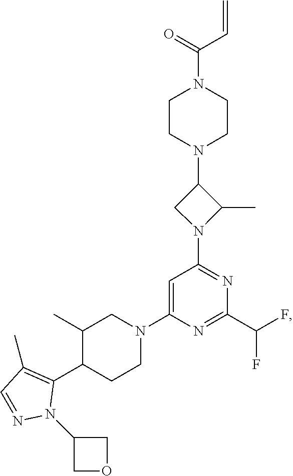 [SMILES: C=CC(=O)N1CCN(C2CN(c3cc(N4CCC(c5c(C)cnn5C5COC5)C(C)C4)nc(C(F)F)n3)C2C)CC1]

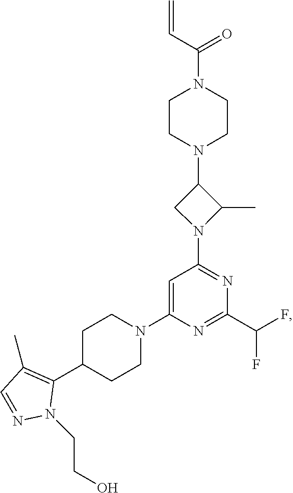 [SMILES: C=CC(=O)N1CCN(C2CN(c3cc(N4CCC(c5c(C)cnn5CCO)CC4)nc(C(F)F)n3)C2C)CC1]

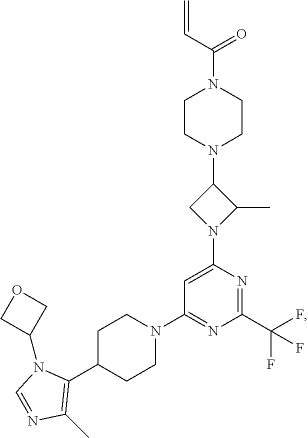 [SMILES: C=CC(=O)N1CCN(C2CN(c3cc(N4CCC(c5c(C)ncn5C5COC5)CC4)nc(C(F)(F)F)n3)C2C)CC1]

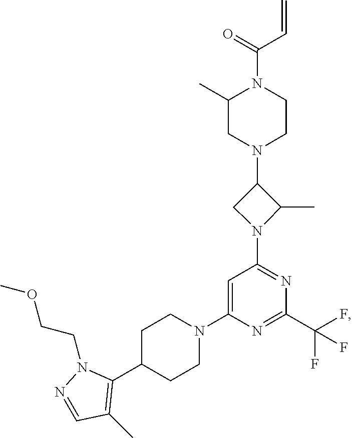 [SMILES: C=CC(=O)N1CCN(C2CN(c3cc(N4CCC(c5c(C)cnn5CCOC)CC4)nc(C(F)(F)F)n3)C2C)CC1C]

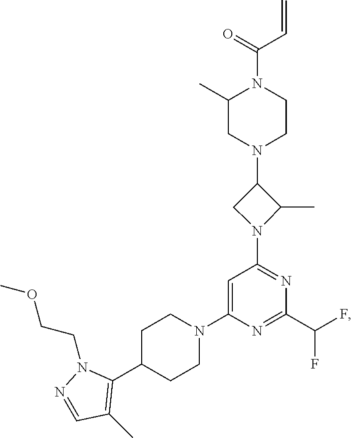 [SMILES: C=CC(=O)N1CCN(C2CN(c3cc(N4CCC(c5c(C)cnn5CCOC)CC4)nc(C(F)F)n3)C2C)CC1C]

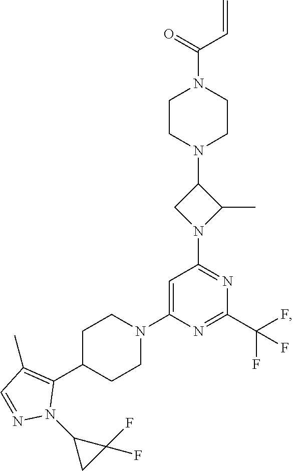 [SMILES: C=CC(=O)N1CCN(C2CN(c3cc(N4CCC(c5c(C)cnn5C5CC5(F)F)CC4)nc(C(F)(F)F)n3)C2C)CC1]

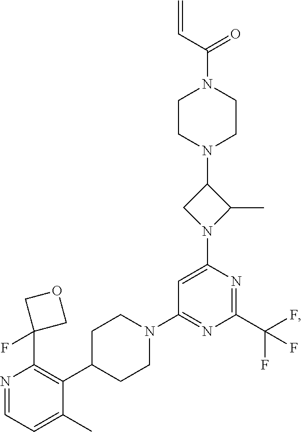 [SMILES: C=CC(=O)N1CCN(C2CN(c3cc(N4CCC(c5c(C)ccnc5C5(F)COC5)CC4)nc(C(F)(F)F)n3)C2C)CC1]

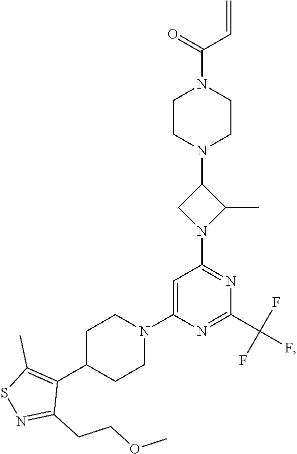 [SMILES: C=CC(=O)N1CCN(C2CN(c3cc(N4CCC(c5c(CCOC)nsc5C)CC4)nc(C(F)(F)F)n3)C2C)CC1]

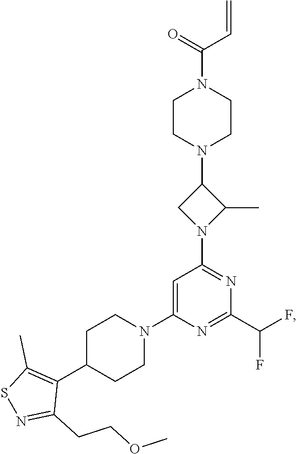 [SMILES: C=CC(=O)N1CCN(C2CN(c3cc(N4CCC(c5c(CCOC)nsc5C)CC4)nc(C(F)F)n3)C2C)CC1]

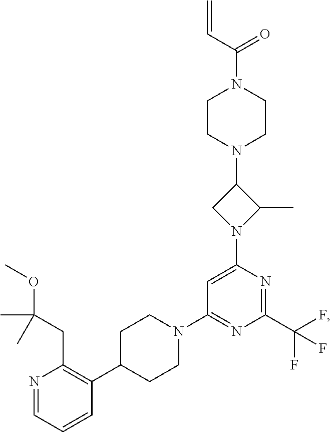 [SMILES: C=CC(=O)N1CCN(C2CN(c3cc(N4CCC(c5cccnc5CC(C)(C)OC)CC4)nc(C(F)(F)F)n3)C2C)CC1]

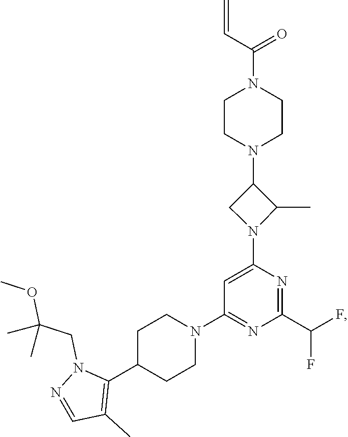 [SMILES: C=CC(=O)N1CCN(C2CN(c3cc(N4CCC(c5c(C)cnn5CC(C)(C)OC)CC4)nc(C(F)F)n3)C2C)CC1]

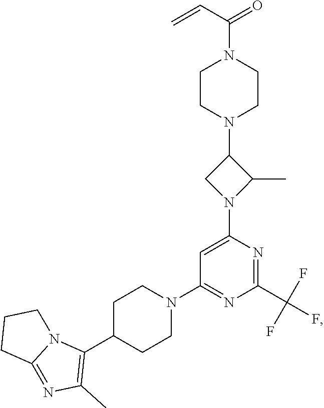 [SMILES: C=CC(=O)N1CCN(C2CN(c3cc(N4CCC(c5c(C)nc6n5CCC6)CC4)nc(C(F)(F)F)n3)C2C)CC1]

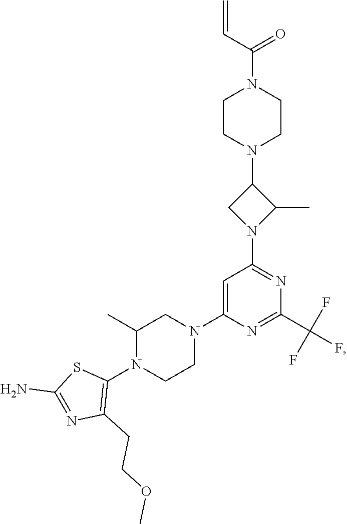 [SMILES: C=CC(=O)N1CCN(C2CN(c3cc(N4CCN(c5sc(N)nc5CCOC)C(C)C4)nc(C(F)(F)F)n3)C2C)CC1]

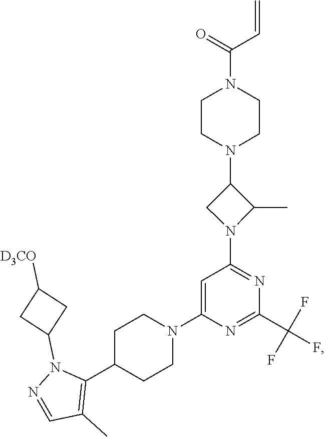

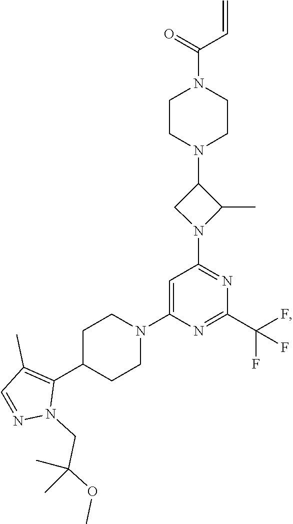 [SMILES: C=CC(=O)N1CCN(C2CN(c3cc(N4CCC(c5c(C)cnn5CC(C)(C)OC)CC4)nc(C(F)(F)F)n3)C2C)CC1]

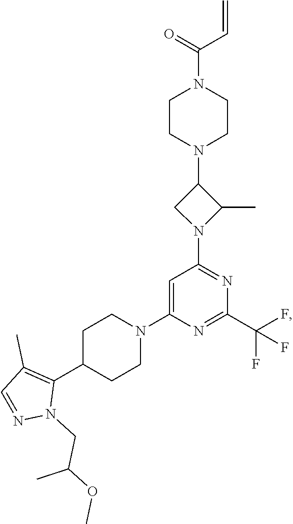 [SMILES: C=CC(=O)N1CCN(C2CN(c3cc(N4CCC(c5c(C)cnn5CC(C)OC)CC4)nc(C(F)(F)F)n3)C2C)CC1]

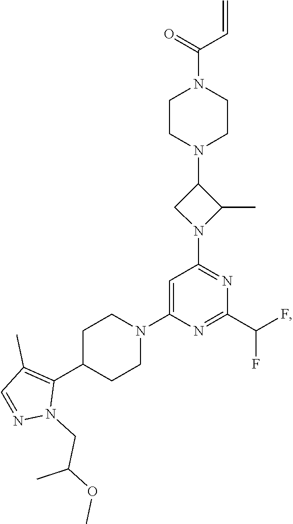 [SMILES: C=CC(=O)N1CCN(C2CN(c3cc(N4CCC(c5c(C)cnn5CC(C)OC)CC4)nc(C(F)F)n3)C2C)CC1]

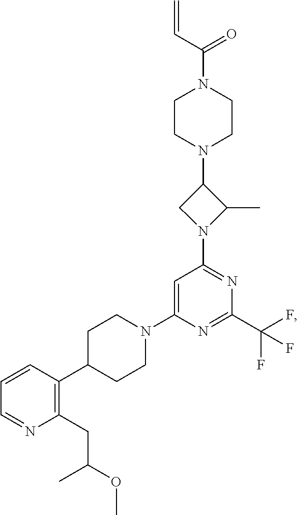 [SMILES: C=CC(=O)N1CCN(C2CN(c3cc(N4CCC(c5cccnc5CC(C)OC)CC4)nc(C(F)(F)F)n3)C2C)CC1]

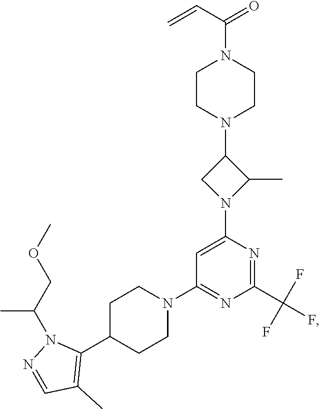 [SMILES: C=CC(=O)N1CCN(C2CN(c3cc(N4CCC(c5c(C)cnn5C(C)COC)CC4)nc(C(F)(F)F)n3)C2C)CC1]

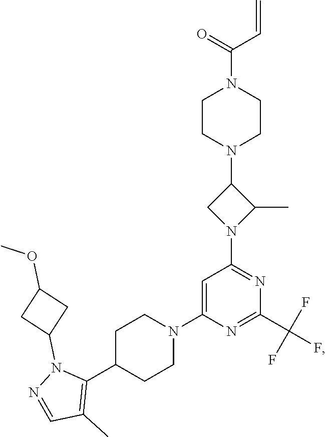 [SMILES: C=CC(=O)N1CCN(C2CN(c3cc(N4CCC(c5c(C)cnn5C5CC(OC)C5)CC4)nc(C(F)(F)F)n3)C2C)CC1]

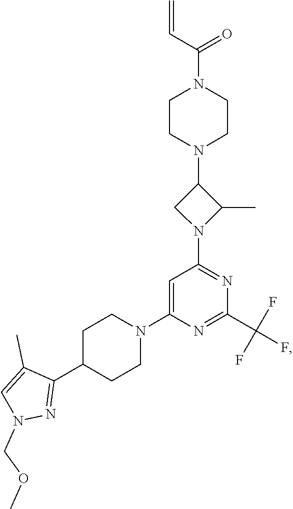 [SMILES: C=CC(=O)N1CCN(C2CN(c3cc(N4CCC(c5nn(COC)cc5C)CC4)nc(C(F)(F)F)n3)C2C)CC1]

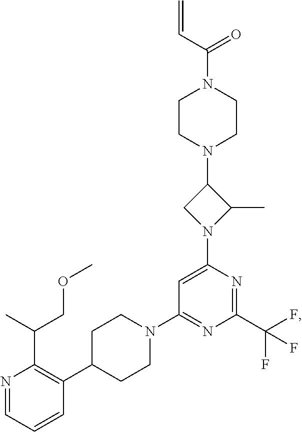 [SMILES: C=CC(=O)N1CCN(C2CN(c3cc(N4CCC(c5cccnc5C(C)COC)CC4)nc(C(F)(F)F)n3)C2C)CC1]

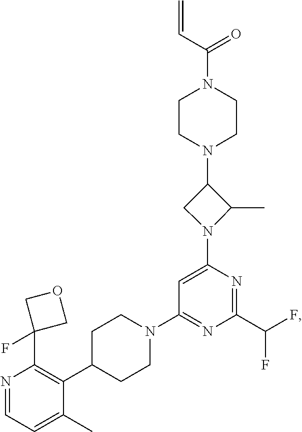 [SMILES: C=CC(=O)N1CCN(C2CN(c3cc(N4CCC(c5c(C)ccnc5C5(F)COC5)CC4)nc(C(F)F)n3)C2C)CC1]

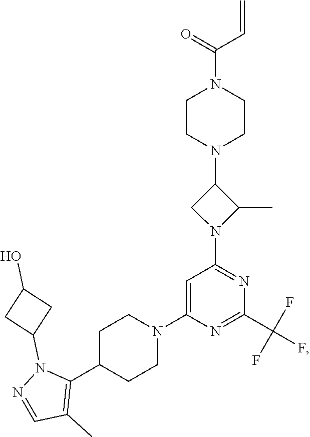 [SMILES: C=CC(=O)N1CCN(C2CN(c3cc(N4CCC(c5c(C)cnn5C5CC(O)C5)CC4)nc(C(F)(F)F)n3)C2C)CC1]

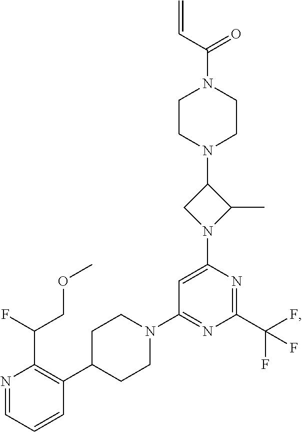 [SMILES: C=CC(=O)N1CCN(C2CN(c3cc(N4CCC(c5cccnc5C(F)COC)CC4)nc(C(F)(F)F)n3)C2C)CC1]

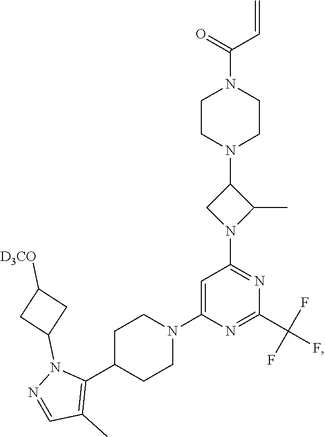

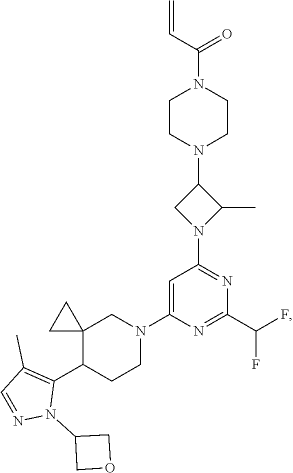 [SMILES: C=CC(=O)N1CCN(C2CN(c3cc(N4CCC(c5c(C)cnn5C5COC5)C5(CC5)C4)nc(C(F)F)n3)C2C)CC1]

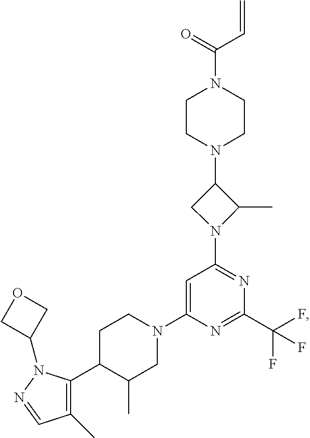 [SMILES: C=CC(=O)N1CCN(C2CN(c3cc(N4CCC(c5c(C)cnn5C5COC5)C(C)C4)nc(C(F)(F)F)n3)C2C)CC1]

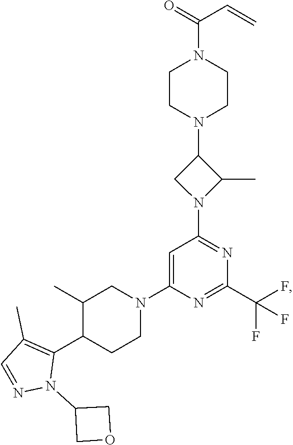 [SMILES: C=CC(=O)N1CCN(C2CN(c3cc(N4CCC(c5c(C)cnn5C5COC5)C(C)C4)nc(C(F)(F)F)n3)C2C)CC1]

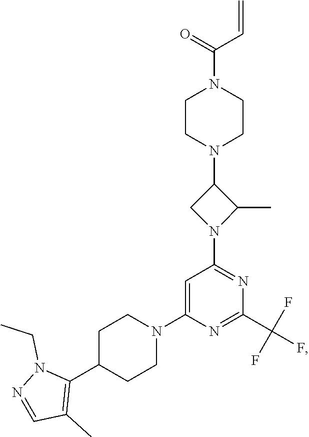 [SMILES: C=CC(=O)N1CCN(C2CN(c3cc(N4CCC(c5c(C)cnn5CC)CC4)nc(C(F)(F)F)n3)C2C)CC1]

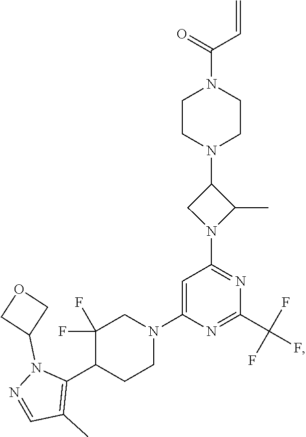 [SMILES: C=CC(=O)N1CCN(C2CN(c3cc(N4CCC(c5c(C)cnn5C5COC5)C(F)(F)C4)nc(C(F)(F)F)n3)C2C)CC1]

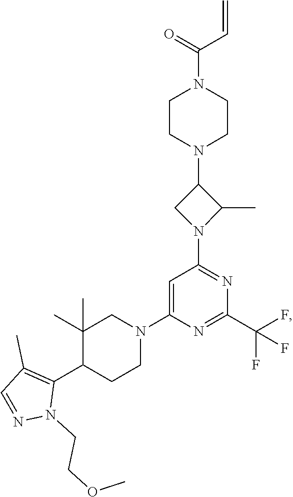 [SMILES: C=CC(=O)N1CCN(C2CN(c3cc(N4CCC(c5c(C)cnn5CCOC)C(C)(C)C4)nc(C(F)(F)F)n3)C2C)CC1]

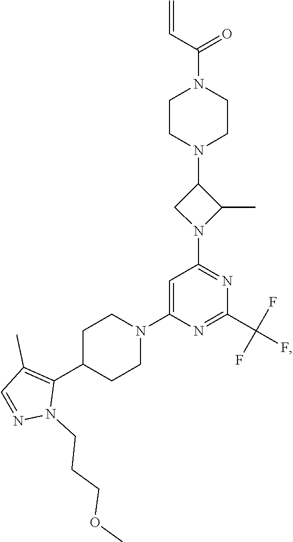 [SMILES: C=CC(=O)N1CCN(C2CN(c3cc(N4CCC(c5c(C)cnn5CCCOC)CC4)nc(C(F)(F)F)n3)C2C)CC1]

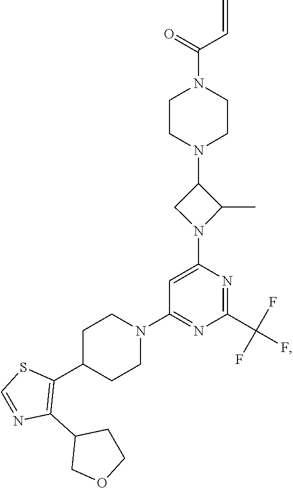 [SMILES: C=CC(=O)N1CCN(C2CN(c3cc(N4CCC(c5scnc5C5CCOC5)CC4)nc(C(F)(F)F)n3)C2C)CC1]

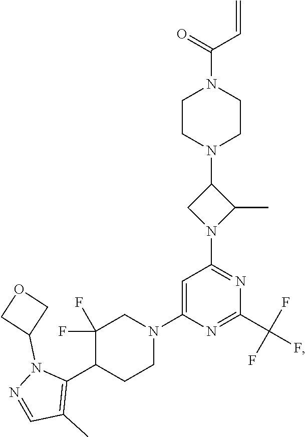 [SMILES: C=CC(=O)N1CCN(C2CN(c3cc(N4CCC(c5c(C)cnn5C5COC5)C(F)(F)C4)nc(C(F)(F)F)n3)C2C)CC1]

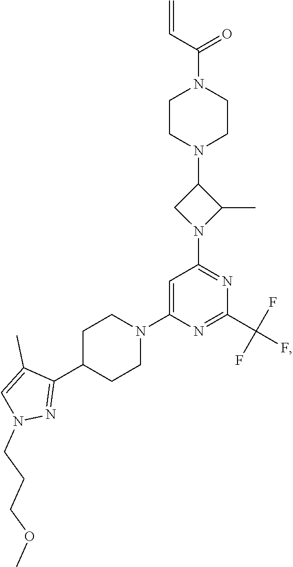 [SMILES: C=CC(=O)N1CCN(C2CN(c3cc(N4CCC(c5nn(CCCOC)cc5C)CC4)nc(C(F)(F)F)n3)C2C)CC1]

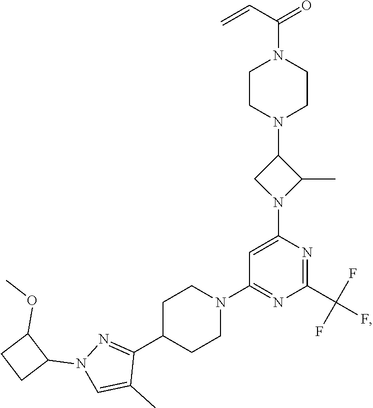 [SMILES: C=CC(=O)N1CCN(C2CN(c3cc(N4CCC(c5nn(C6CCC6OC)cc5C)CC4)nc(C(F)(F)F)n3)C2C)CC1]

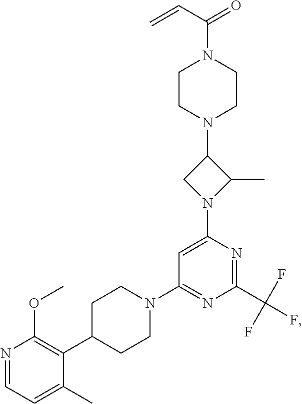 [SMILES: C=CC(=O)N1CCN(C2CN(c3cc(N4CCC(c5c(C)ccnc5OC)CC4)nc(C(F)(F)F)n3)C2C)CC1]

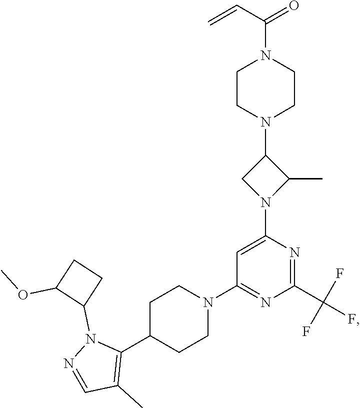 [SMILES: C=CC(=O)N1CCN(C2CN(c3cc(N4CCC(c5c(C)cnn5C5CCC5OC)CC4)nc(C(F)(F)F)n3)C2C)CC1]

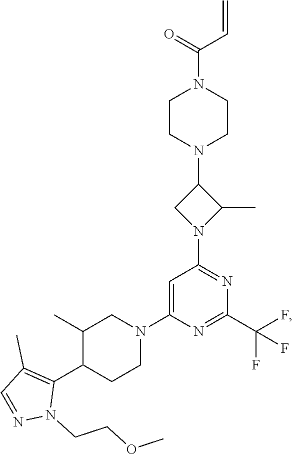 [SMILES: C=CC(=O)N1CCN(C2CN(c3cc(N4CCC(c5c(C)cnn5CCOC)C(C)C4)nc(C(F)(F)F)n3)C2C)CC1]

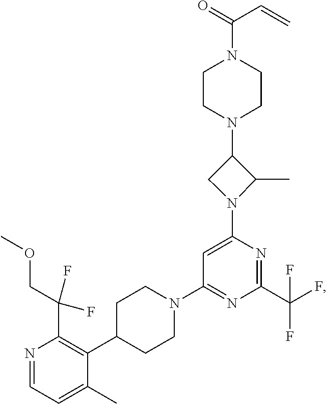 [SMILES: C=CC(=O)N1CCN(C2CN(c3cc(N4CCC(c5c(C)ccnc5C(F)(F)COC)CC4)nc(C(F)(F)F)n3)C2C)CC1]

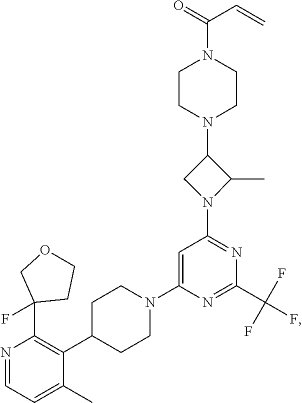 [SMILES: C=CC(=O)N1CCN(C2CN(c3cc(N4CCC(c5c(C)ccnc5C5(F)CCOC5)CC4)nc(C(F)(F)F)n3)C2C)CC1]

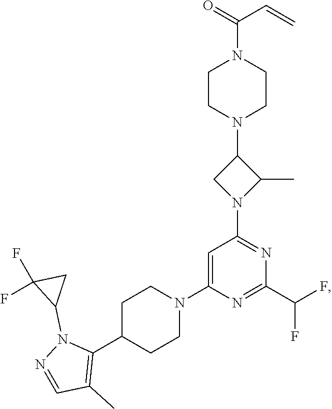 [SMILES: C=CC(=O)N1CCN(C2CN(c3cc(N4CCC(c5c(C)cnn5C5CC5(F)F)CC4)nc(C(F)F)n3)C2C)CC1]

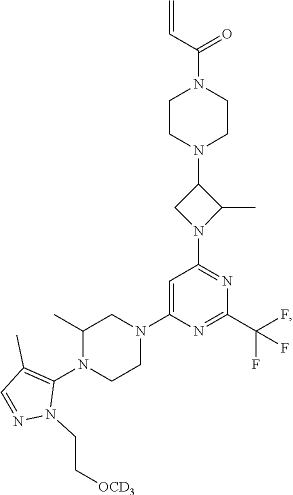

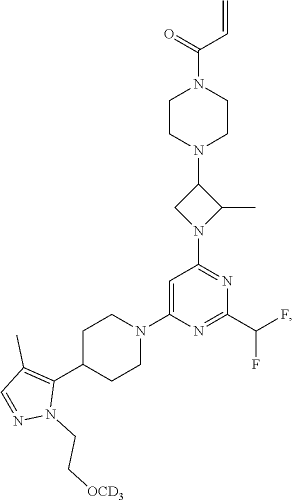

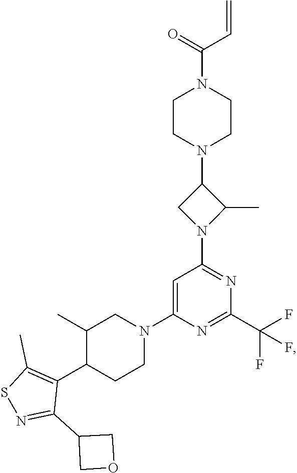 [SMILES: C=CC(=O)N1CCN(C2CN(c3cc(N4CCC(c5c(C6COC6)nsc5C)C(C)C4)nc(C(F)(F)F)n3)C2C)CC1]

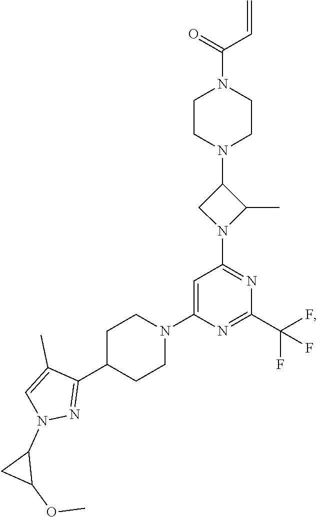 [SMILES: C=CC(=O)N1CCN(C2CN(c3cc(N4CCC(c5nn(C6CC6OC)cc5C)CC4)nc(C(F)(F)F)n3)C2C)CC1]

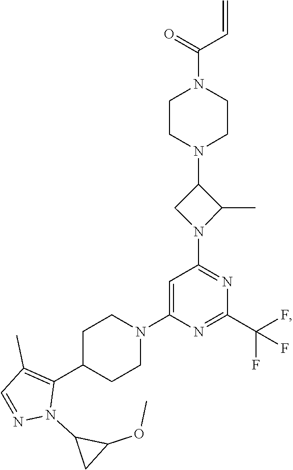 [SMILES: C=CC(=O)N1CCN(C2CN(c3cc(N4CCC(c5c(C)cnn5C5CC5OC)CC4)nc(C(F)(F)F)n3)C2C)CC1]

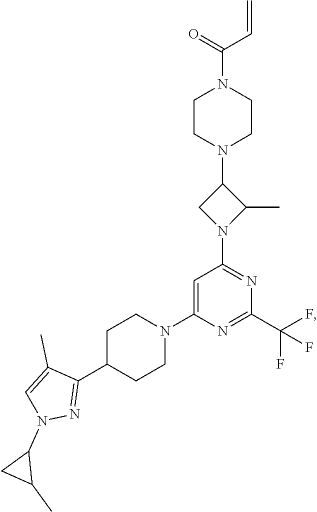 [SMILES: C=CC(=O)N1CCN(C2CN(c3cc(N4CCC(c5nn(C6CC6C)cc5C)CC4)nc(C(F)(F)F)n3)C2C)CC1]

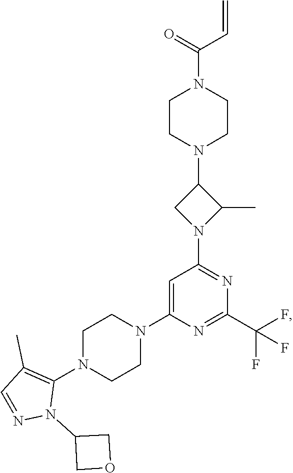 [SMILES: C=CC(=O)N1CCN(C2CN(c3cc(N4CCN(c5c(C)cnn5C5COC5)CC4)nc(C(F)(F)F)n3)C2C)CC1]

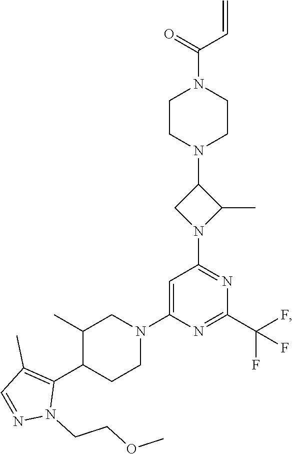 [SMILES: C=CC(=O)N1CCN(C2CN(c3cc(N4CCC(c5c(C)cnn5CCOC)C(C)C4)nc(C(F)(F)F)n3)C2C)CC1]

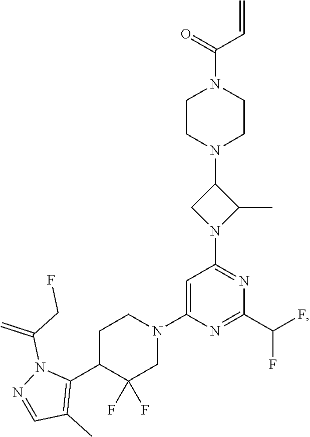 [SMILES: C=CC(=O)N1CCN(C2CN(c3cc(N4CCC(c5c(C)cnn5C(=C)CF)C(F)(F)C4)nc(C(F)F)n3)C2C)CC1]

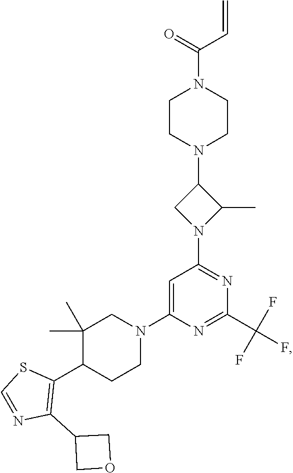 [SMILES: C=CC(=O)N1CCN(C2CN(c3cc(N4CCC(c5scnc5C5COC5)C(C)(C)C4)nc(C(F)(F)F)n3)C2C)CC1]

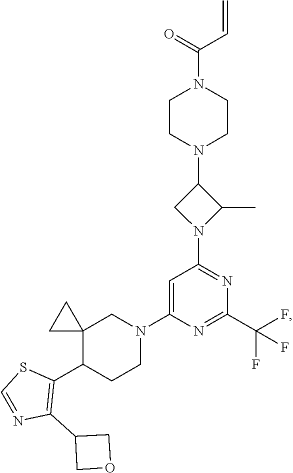 [SMILES: C=CC(=O)N1CCN(C2CN(c3cc(N4CCC(c5scnc5C5COC5)C5(CC5)C4)nc(C(F)(F)F)n3)C2C)CC1]

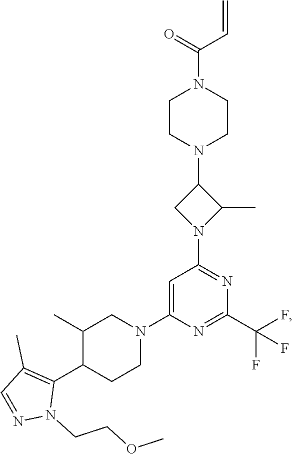 [SMILES: C=CC(=O)N1CCN(C2CN(c3cc(N4CCC(c5c(C)cnn5CCOC)C(C)C4)nc(C(F)(F)F)n3)C2C)CC1]

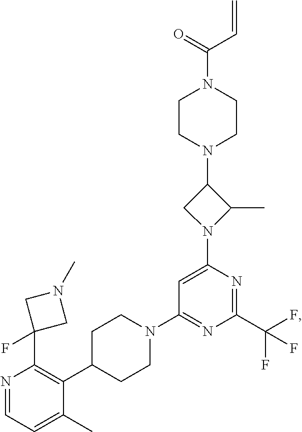 [SMILES: C=CC(=O)N1CCN(C2CN(c3cc(N4CCC(c5c(C)ccnc5C5(F)CN(C)C5)CC4)nc(C(F)(F)F)n3)C2C)CC1]

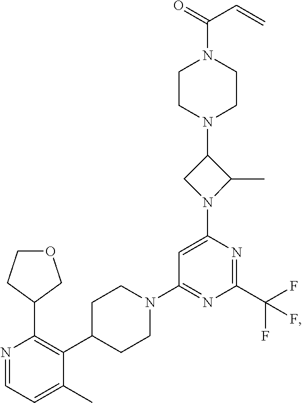 [SMILES: C=CC(=O)N1CCN(C2CN(c3cc(N4CCC(c5c(C)ccnc5C5CCOC5)CC4)nc(C(F)(F)F)n3)C2C)CC1]

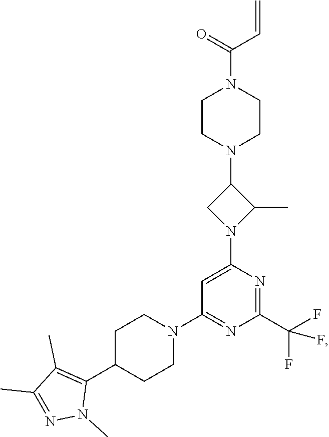 [SMILES: C=CC(=O)N1CCN(C2CN(c3cc(N4CCC(c5c(C)c(C)nn5C)CC4)nc(C(F)(F)F)n3)C2C)CC1]

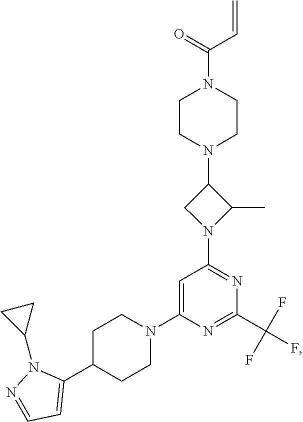 [SMILES: C=CC(=O)N1CCN(C2CN(c3cc(N4CCC(c5ccnn5C5CC5)CC4)nc(C(F)(F)F)n3)C2C)CC1]

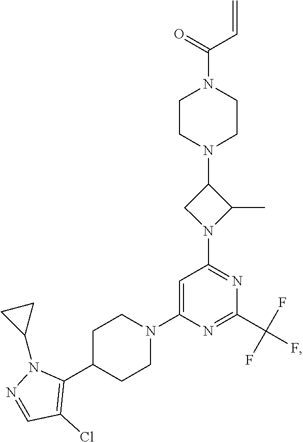 [SMILES: C=CC(=O)N1CCN(C2CN(c3cc(N4CCC(c5c(Cl)cnn5C5CC5)CC4)nc(C(F)(F)F)n3)C2C)CC1]

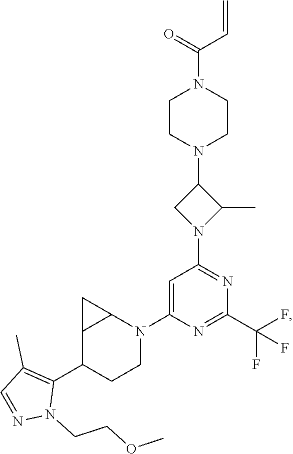 [SMILES: C=CC(=O)N1CCN(C2CN(c3cc(N4CCC(c5c(C)cnn5CCOC)C5CC54)nc(C(F)(F)F)n3)C2C)CC1]

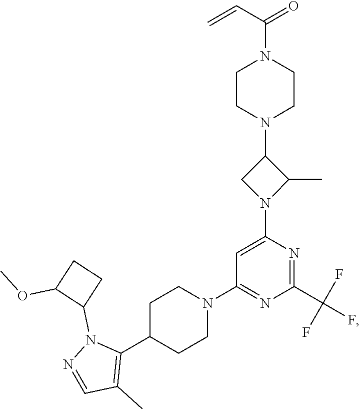 [SMILES: C=CC(=O)N1CCN(C2CN(c3cc(N4CCC(c5c(C)cnn5C5CCC5OC)CC4)nc(C(F)(F)F)n3)C2C)CC1]

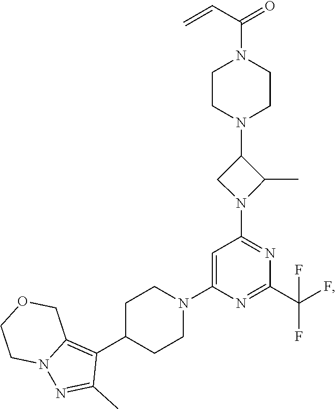 [SMILES: C=CC(=O)N1CCN(C2CN(c3cc(N4CCC(c5c(C)nn6c5COCC6)CC4)nc(C(F)(F)F)n3)C2C)CC1]

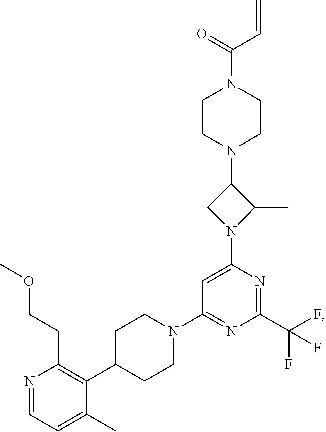 [SMILES: C=CC(=O)N1CCN(C2CN(c3cc(N4CCC(c5c(C)ccnc5CCOC)CC4)nc(C(F)(F)F)n3)C2C)CC1]

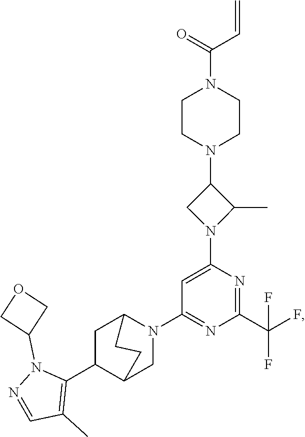 [SMILES: C=CC(=O)N1CCN(C2CN(c3cc(N4CC5CCC4CC5c4c(C)cnn4C4COC4)nc(C(F)(F)F)n3)C2C)CC1]

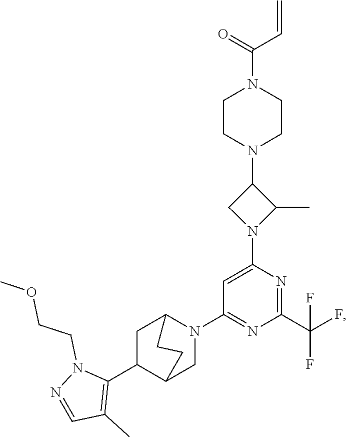 [SMILES: C=CC(=O)N1CCN(C2CN(c3cc(N4CC5CCC4CC5c4c(C)cnn4CCOC)nc(C(F)(F)F)n3)C2C)CC1]

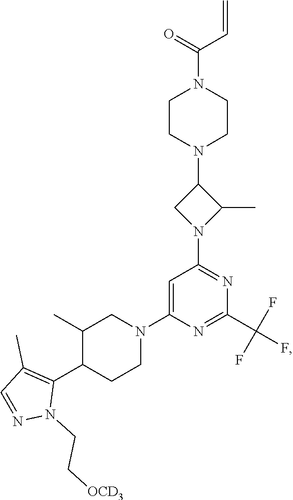

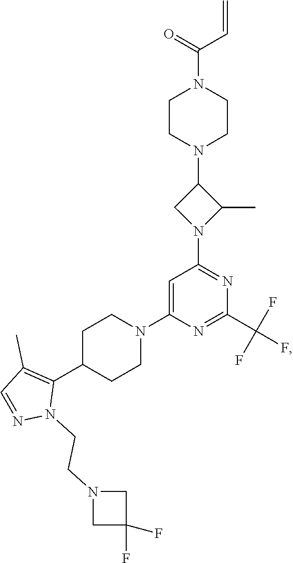 [SMILES: C=CC(=O)N1CCN(C2CN(c3cc(N4CCC(c5c(C)cnn5CCN5CC(F)(F)C5)CC4)nc(C(F)(F)F)n3)C2C)CC1]

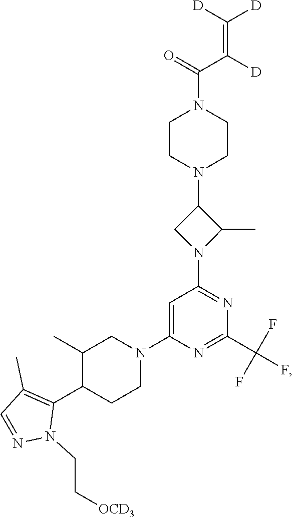

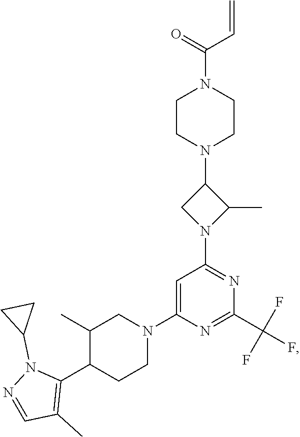 [SMILES: C=CC(=O)N1CCN(C2CN(c3cc(N4CCC(c5c(C)cnn5C5CC5)C(C)C4)nc(C(F)(F)F)n3)C2C)CC1]

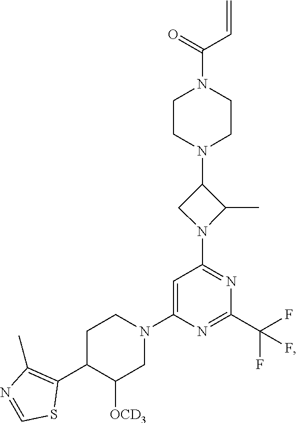

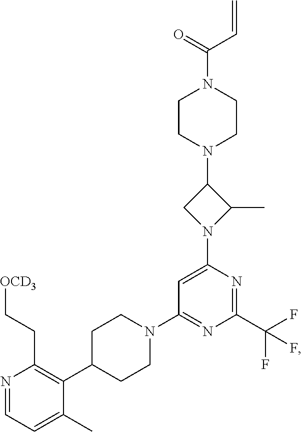

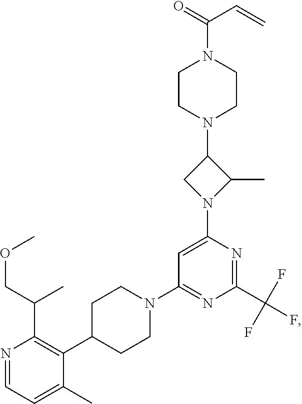 [SMILES: C=CC(=O)N1CCN(C2CN(c3cc(N4CCC(c5c(C)ccnc5C(C)COC)CC4)nc(C(F)(F)F)n3)C2C)CC1]

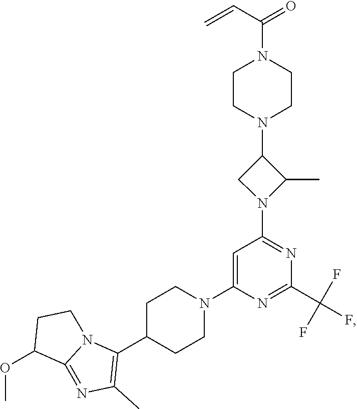 [SMILES: C=CC(=O)N1CCN(C2CN(c3cc(N4CCC(c5c(C)nc6n5CCC6OC)CC4)nc(C(F)(F)F)n3)C2C)CC1]

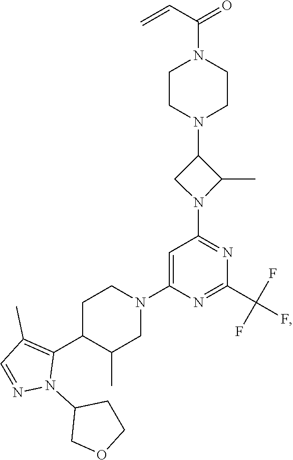 [SMILES: C=CC(=O)N1CCN(C2CN(c3cc(N4CCC(c5c(C)cnn5C5CCOC5)C(C)C4)nc(C(F)(F)F)n3)C2C)CC1]

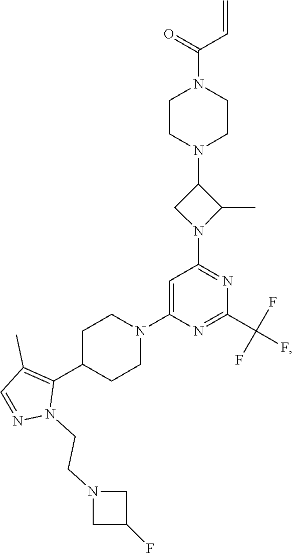 [SMILES: C=CC(=O)N1CCN(C2CN(c3cc(N4CCC(c5c(C)cnn5CCN5CC(F)C5)CC4)nc(C(F)(F)F)n3)C2C)CC1]

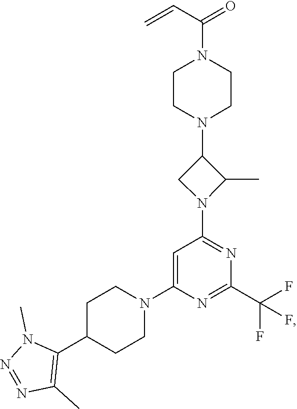 [SMILES: C=CC(=O)N1CCN(C2CN(c3cc(N4CCC(c5c(C)nnn5C)CC4)nc(C(F)(F)F)n3)C2C)CC1]

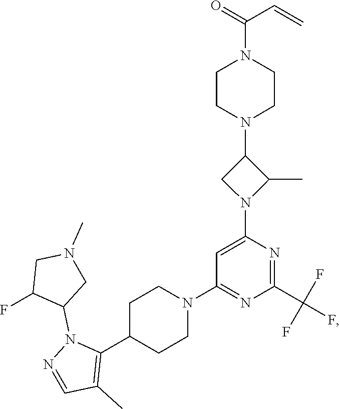 [SMILES: C=CC(=O)N1CCN(C2CN(c3cc(N4CCC(c5c(C)cnn5C5CN(C)CC5F)CC4)nc(C(F)(F)F)n3)C2C)CC1]

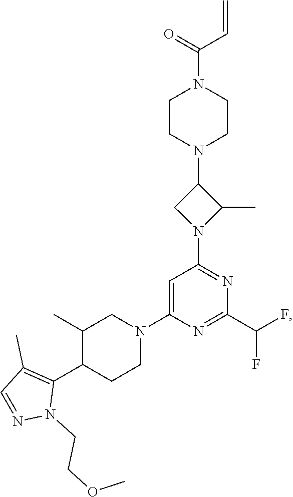 [SMILES: C=CC(=O)N1CCN(C2CN(c3cc(N4CCC(c5c(C)cnn5CCOC)C(C)C4)nc(C(F)F)n3)C2C)CC1]

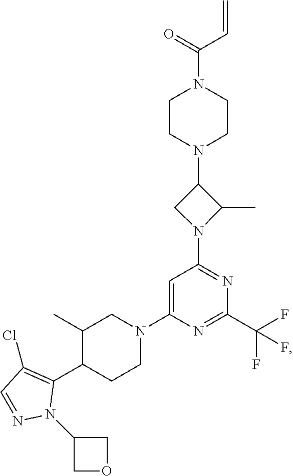 [SMILES: C=CC(=O)N1CCN(C2CN(c3cc(N4CCC(c5c(Cl)cnn5C5COC5)C(C)C4)nc(C(F)(F)F)n3)C2C)CC1]

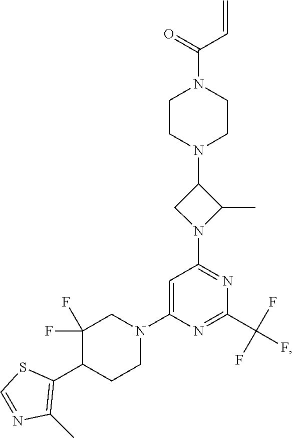 [SMILES: C=CC(=O)N1CCN(C2CN(c3cc(N4CCC(c5scnc5C)C(F)(F)C4)nc(C(F)(F)F)n3)C2C)CC1]

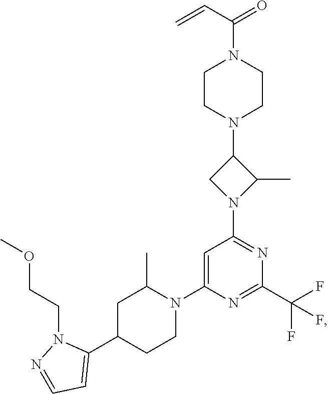 [SMILES: C=CC(=O)N1CCN(C2CN(c3cc(N4CCC(c5ccnn5CCOC)CC4C)nc(C(F)(F)F)n3)C2C)CC1]

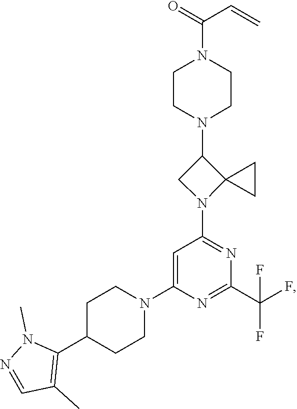 [SMILES: C=CC(=O)N1CCN(C2CN(c3cc(N4CCC(c5c(C)cnn5C)CC4)nc(C(F)(F)F)n3)C23CC3)CC1]

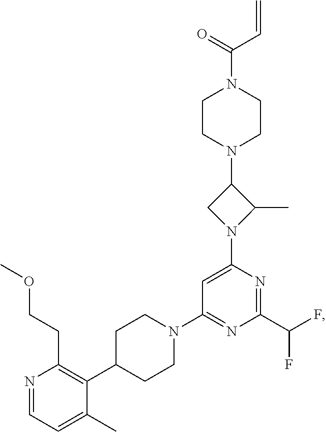 [SMILES: C=CC(=O)N1CCN(C2CN(c3cc(N4CCC(c5c(C)ccnc5CCOC)CC4)nc(C(F)F)n3)C2C)CC1]

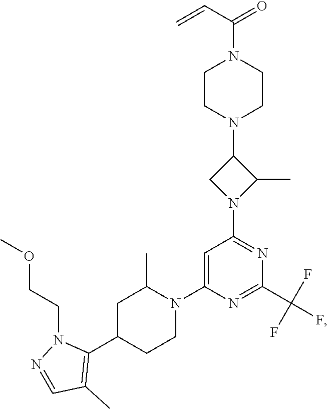 [SMILES: C=CC(=O)N1CCN(C2CN(c3cc(N4CCC(c5c(C)cnn5CCOC)CC4C)nc(C(F)(F)F)n3)C2C)CC1]

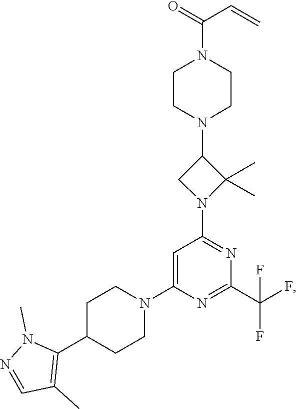 [SMILES: C=CC(=O)N1CCN(C2CN(c3cc(N4CCC(c5c(C)cnn5C)CC4)nc(C(F)(F)F)n3)C2(C)C)CC1]

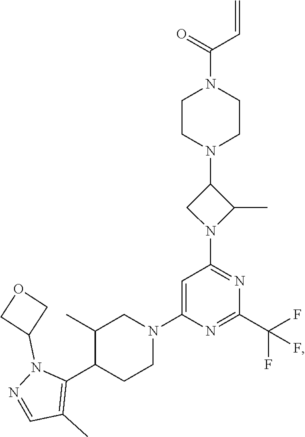 [SMILES: C=CC(=O)N1CCN(C2CN(c3cc(N4CCC(c5c(C)cnn5C5COC5)C(C)C4)nc(C(F)(F)F)n3)C2C)CC1]

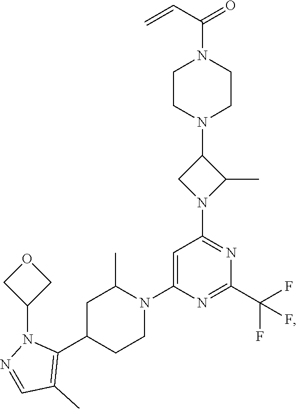 [SMILES: C=CC(=O)N1CCN(C2CN(c3cc(N4CCC(c5c(C)cnn5C5COC5)CC4C)nc(C(F)(F)F)n3)C2C)CC1]

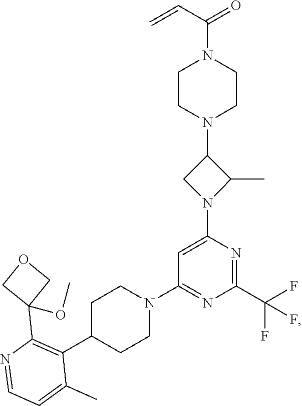 [SMILES: C=CC(=O)N1CCN(C2CN(c3cc(N4CCC(c5c(C)ccnc5C5(OC)COC5)CC4)nc(C(F)(F)F)n3)C2C)CC1]

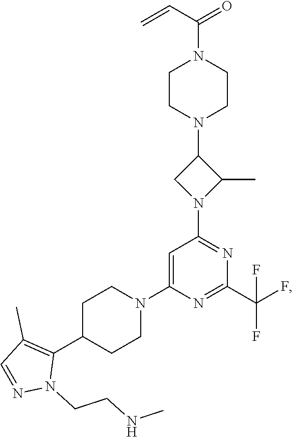 [SMILES: C=CC(=O)N1CCN(C2CN(c3cc(N4CCC(c5c(C)cnn5CCNC)CC4)nc(C(F)(F)F)n3)C2C)CC1]

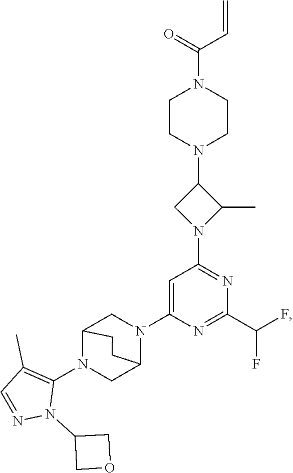 [SMILES: C=CC(=O)N1CCN(C2CN(c3cc(N4CC5CCC4CN5c4c(C)cnn4C4COC4)nc(C(F)F)n3)C2C)CC1]

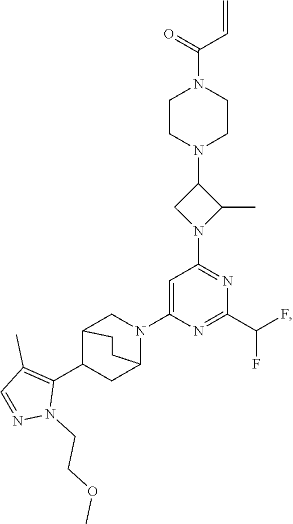 [SMILES: C=CC(=O)N1CCN(C2CN(c3cc(N4CC5CCC4CC5c4c(C)cnn4CCOC)nc(C(F)F)n3)C2C)CC1]

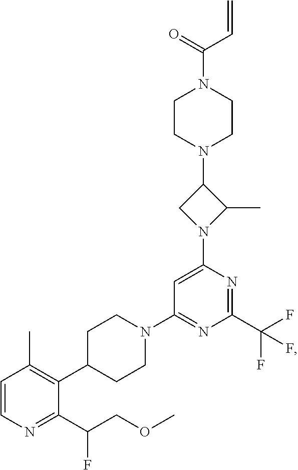 [SMILES: C=CC(=O)N1CCN(C2CN(c3cc(N4CCC(c5c(C)ccnc5C(F)COC)CC4)nc(C(F)(F)F)n3)C2C)CC1]

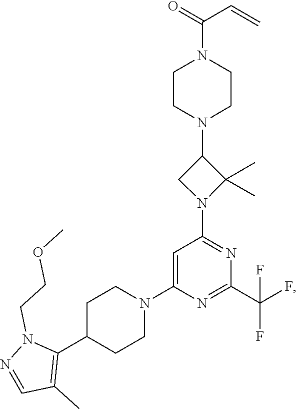 [SMILES: C=CC(=O)N1CCN(C2CN(c3cc(N4CCC(c5c(C)cnn5CCOC)CC4)nc(C(F)(F)F)n3)C2(C)C)CC1]

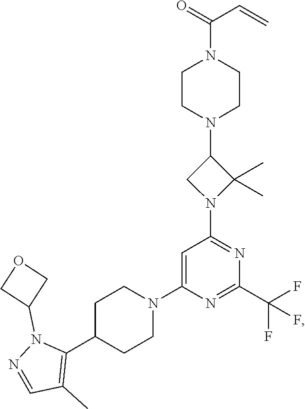 [SMILES: C=CC(=O)N1CCN(C2CN(c3cc(N4CCC(c5c(C)cnn5C5COC5)CC4)nc(C(F)(F)F)n3)C2(C)C)CC1]

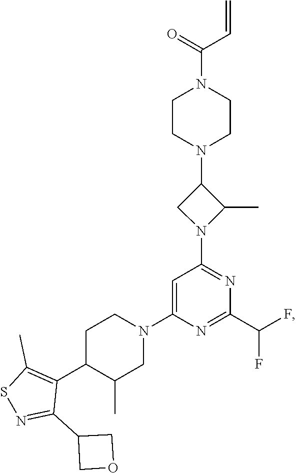 [SMILES: C=CC(=O)N1CCN(C2CN(c3cc(N4CCC(c5c(C6COC6)nsc5C)C(C)C4)nc(C(F)F)n3)C2C)CC1]

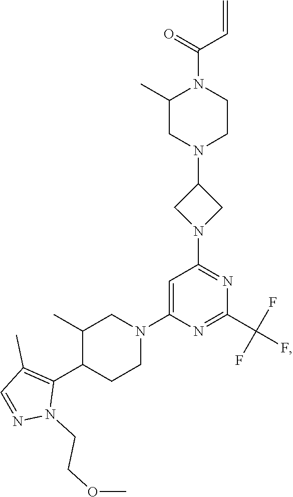 [SMILES: C=CC(=O)N1CCN(C2CN(c3cc(N4CCC(c5c(C)cnn5CCOC)C(C)C4)nc(C(F)(F)F)n3)C2)CC1C]

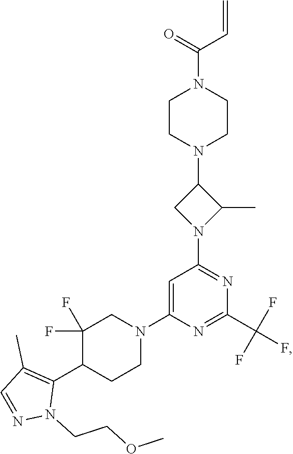 [SMILES: C=CC(=O)N1CCN(C2CN(c3cc(N4CCC(c5c(C)cnn5CCOC)C(F)(F)C4)nc(C(F)(F)F)n3)C2C)CC1]

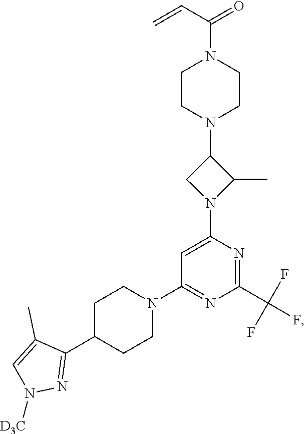 [SMILES: [2H]C([2H])([2H])n1cc(C)c(C2CCN(c3cc(N4CC(N5CCN(C(=O)C=C)CC5)C4C)nc(C(F)(F)F)n3)CC2)n1]

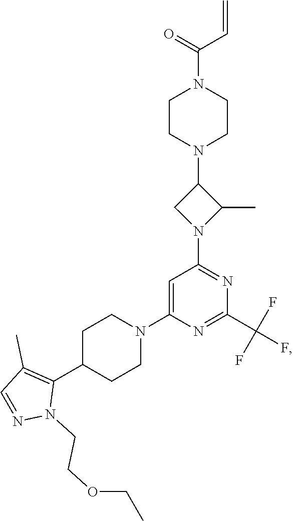 [SMILES: C=CC(=O)N1CCN(C2CN(c3cc(N4CCC(c5c(C)cnn5CCOCC)CC4)nc(C(F)(F)F)n3)C2C)CC1]

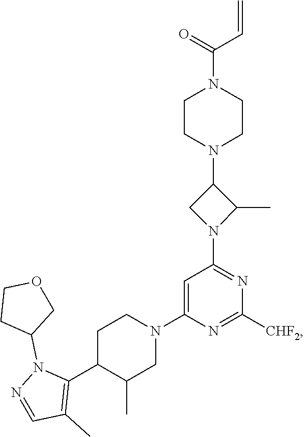 [SMILES: C=CC(=O)N1CCN(C2CN(c3cc(N4CCC(c5c(C)cnn5C5CCOC5)C(C)C4)nc(C(F)F)n3)C2C)CC1]

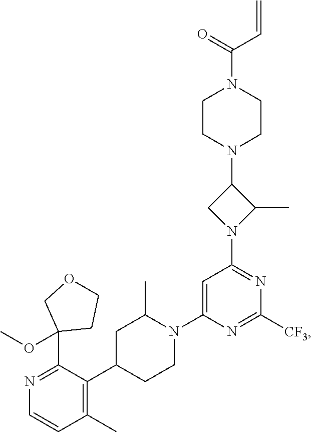 [SMILES: C=CC(=O)N1CCN(C2CN(c3cc(N4CCC(c5c(C)ccnc5C5(OC)CCOC5)CC4C)nc(C(F)(F)F)n3)C2C)CC1]

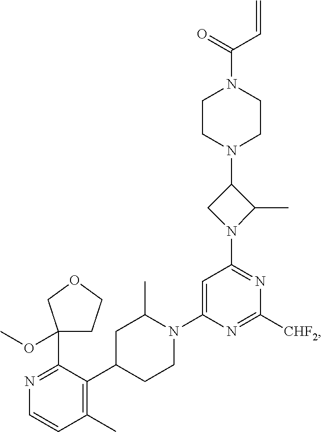 [SMILES: C=CC(=O)N1CCN(C2CN(c3cc(N4CCC(c5c(C)ccnc5C5(OC)CCOC5)CC4C)nc(C(F)F)n3)C2C)CC1]

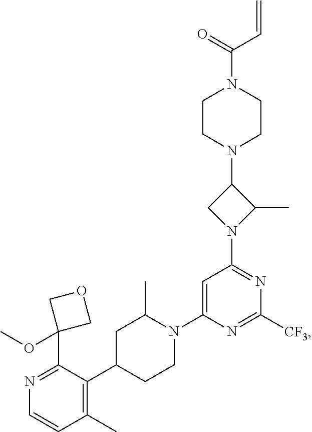 [SMILES: C=CC(=O)N1CCN(C2CN(c3cc(N4CCC(c5c(C)ccnc5C5(OC)COC5)CC4C)nc(C(F)(F)F)n3)C2C)CC1]

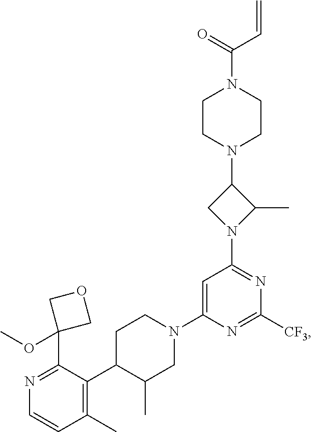 [SMILES: C=CC(=O)N1CCN(C2CN(c3cc(N4CCC(c5c(C)ccnc5C5(OC)COC5)C(C)C4)nc(C(F)(F)F)n3)C2C)CC1]

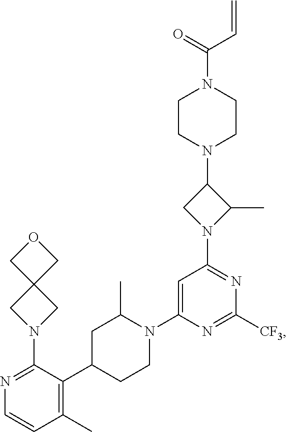 [SMILES: C=CC(=O)N1CCN(C2CN(c3cc(N4CCC(c5c(C)ccnc5N5CC6(COC6)C5)CC4C)nc(C(F)(F)F)n3)C2C)CC1]

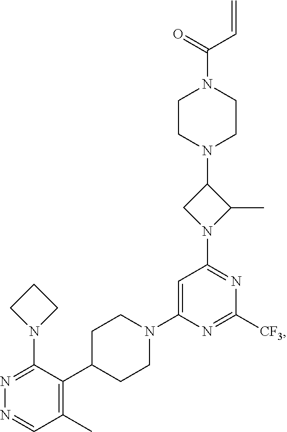 [SMILES: C=CC(=O)N1CCN(C2CN(c3cc(N4CCC(c5c(C)cnnc5N5CCC5)CC4)nc(C(F)(F)F)n3)C2C)CC1]

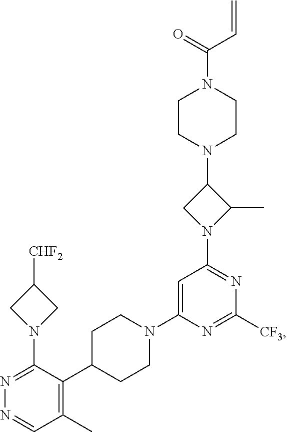 [SMILES: C=CC(=O)N1CCN(C2CN(c3cc(N4CCC(c5c(C)cnnc5N5CC(C(F)F)C5)CC4)nc(C(F)(F)F)n3)C2C)CC1]

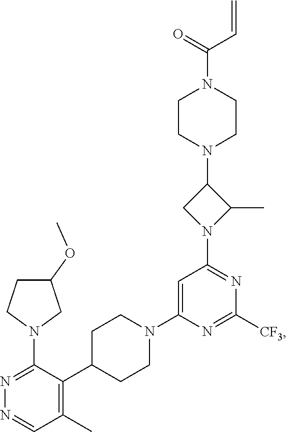 [SMILES: C=CC(=O)N1CCN(C2CN(c3cc(N4CCC(c5c(C)cnnc5N5CCC(OC)C5)CC4)nc(C(F)(F)F)n3)C2C)CC1]

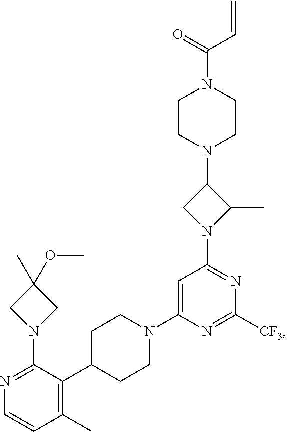 [SMILES: C=CC(=O)N1CCN(C2CN(c3cc(N4CCC(c5c(C)ccnc5N5CC(C)(OC)C5)CC4)nc(C(F)(F)F)n3)C2C)CC1]

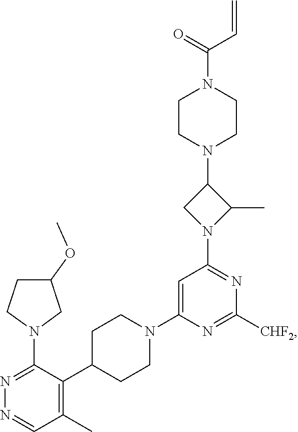 [SMILES: C=CC(=O)N1CCN(C2CN(c3cc(N4CCC(c5c(C)cnnc5N5CCC(OC)C5)CC4)nc(C(F)F)n3)C2C)CC1]

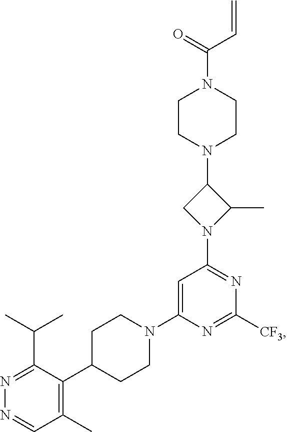 [SMILES: C=CC(=O)N1CCN(C2CN(c3cc(N4CCC(c5c(C)cnnc5C(C)C)CC4)nc(C(F)(F)F)n3)C2C)CC1]

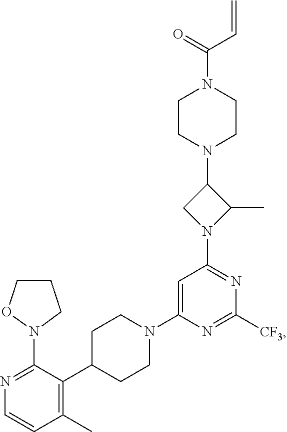 [SMILES: C=CC(=O)N1CCN(C2CN(c3cc(N4CCC(c5c(C)ccnc5N5CCCO5)CC4)nc(C(F)(F)F)n3)C2C)CC1]

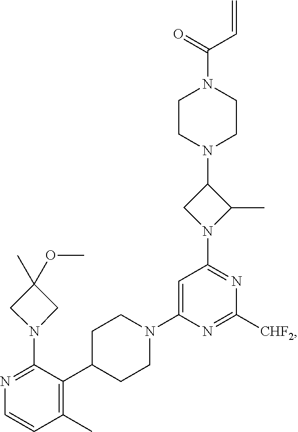 [SMILES: C=CC(=O)N1CCN(C2CN(c3cc(N4CCC(c5c(C)ccnc5N5CC(C)(OC)C5)CC4)nc(C(F)F)n3)C2C)CC1]

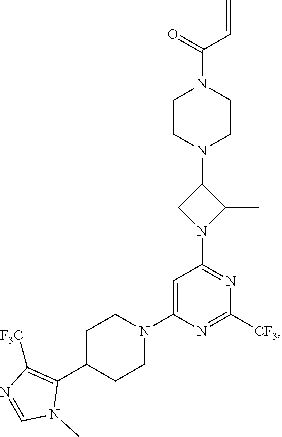 [SMILES: C=CC(=O)N1CCN(C2CN(c3cc(N4CCC(c5c(C(F)(F)F)ncn5C)CC4)nc(C(F)(F)F)n3)C2C)CC1]

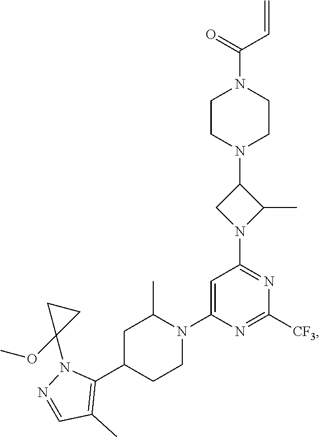 [SMILES: C=CC(=O)N1CCN(C2CN(c3cc(N4CCC(c5c(C)cnn5C5(OC)CC5)CC4C)nc(C(F)(F)F)n3)C2C)CC1]

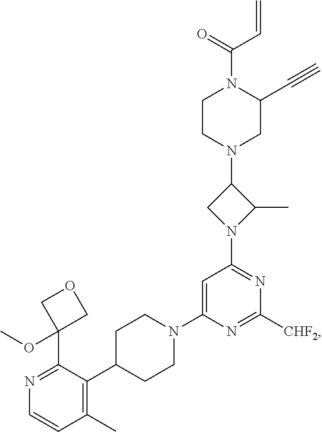 [SMILES: C#CC1CN(C2CN(c3cc(N4CCC(c5c(C)ccnc5C5(OC)COC5)CC4)nc(C(F)F)n3)C2C)CCN1C(=O)C=C]

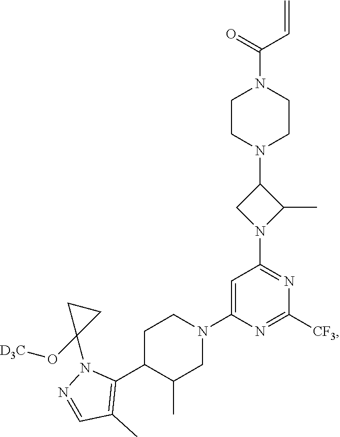 [SMILES: [2H]C([2H])([2H])OC1(n2ncc(C)c2C2CCN(c3cc(N4CC(N5CCN(C(=O)C=C)CC5)C4C)nc(C(F)(F)F)n3)CC2C)CC1]

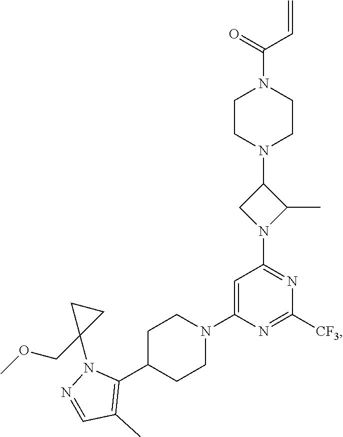 [SMILES: C=CC(=O)N1CCN(C2CN(c3cc(N4CCC(c5c(C)cnn5C5(COC)CC5)CC4)nc(C(F)(F)F)n3)C2C)CC1]

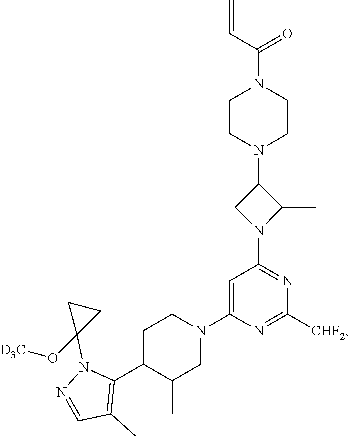 [SMILES: [2H]C([2H])([2H])OC1(n2ncc(C)c2C2CCN(c3cc(N4CC(N5CCN(C(=O)C=C)CC5)C4C)nc(C(F)F)n3)CC2C)CC1]

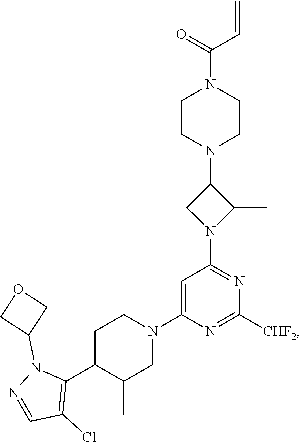 [SMILES: C=CC(=O)N1CCN(C2CN(c3cc(N4CCC(c5c(Cl)cnn5C5COC5)C(C)C4)nc(C(F)F)n3)C2C)CC1]

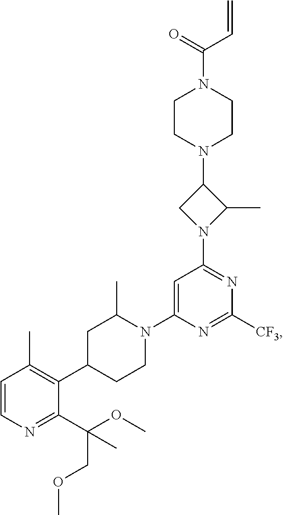 [SMILES: C=CC(=O)N1CCN(C2CN(c3cc(N4CCC(c5c(C)ccnc5C(C)(COC)OC)CC4C)nc(C(F)(F)F)n3)C2C)CC1]

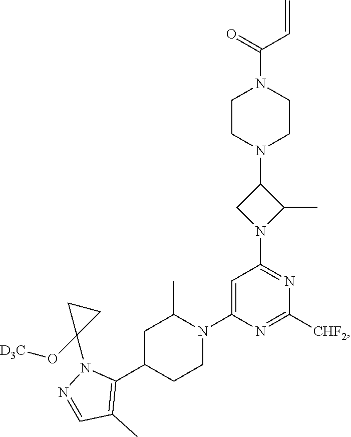 [SMILES: [2H]C([2H])([2H])OC1(n2ncc(C)c2C2CCN(c3cc(N4CC(N5CCN(C(=O)C=C)CC5)C4C)nc(C(F)F)n3)C(C)C2)CC1]

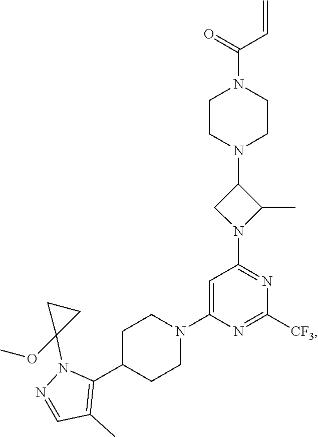 [SMILES: C=CC(=O)N1CCN(C2CN(c3cc(N4CCC(c5c(C)cnn5C5(OC)CC5)CC4)nc(C(F)(F)F)n3)C2C)CC1]

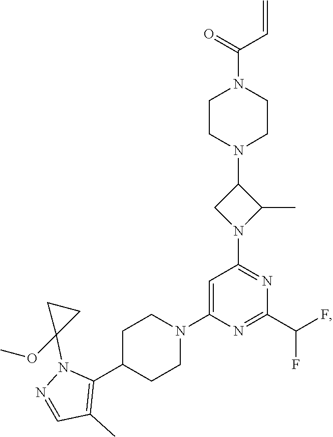 [SMILES: C=CC(=O)N1CCN(C2CN(c3cc(N4CCC(c5c(C)cnn5C5(OC)CC5)CC4)nc(C(F)F)n3)C2C)CC1]

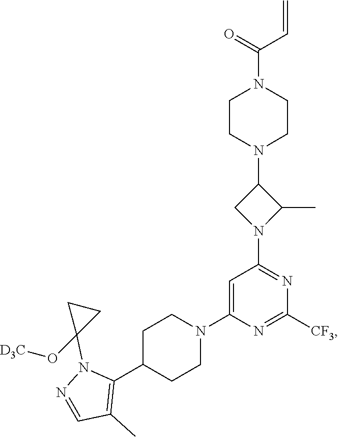 [SMILES: [2H]C([2H])([2H])OC1(n2ncc(C)c2C2CCN(c3cc(N4CC(N5CCN(C(=O)C=C)CC5)C4C)nc(C(F)(F)F)n3)CC2)CC1]

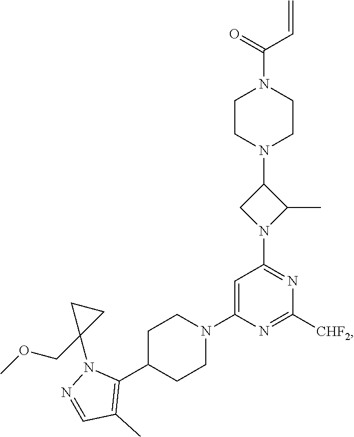 [SMILES: C=CC(=O)N1CCN(C2CN(c3cc(N4CCC(c5c(C)cnn5C5(COC)CC5)CC4)nc(C(F)F)n3)C2C)CC1]

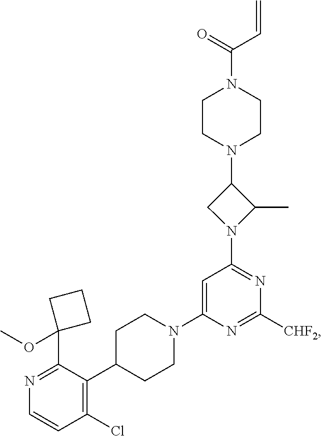 [SMILES: C=CC(=O)N1CCN(C2CN(c3cc(N4CCC(c5c(Cl)ccnc5C5(OC)CCC5)CC4)nc(C(F)F)n3)C2C)CC1]

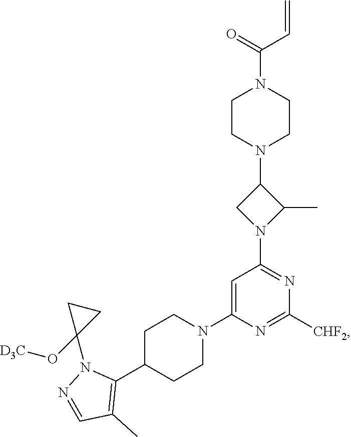 [SMILES: [2H]C([2H])([2H])OC1(n2ncc(C)c2C2CCN(c3cc(N4CC(N5CCN(C(=O)C=C)CC5)C4C)nc(C(F)F)n3)CC2)CC1]

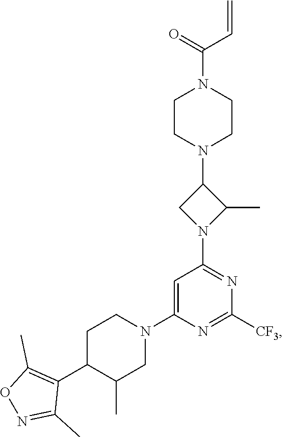 [SMILES: C=CC(=O)N1CCN(C2CN(c3cc(N4CCC(c5c(C)noc5C)C(C)C4)nc(C(F)(F)F)n3)C2C)CC1]

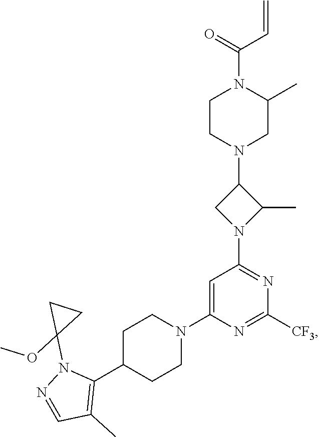 [SMILES: C=CC(=O)N1CCN(C2CN(c3cc(N4CCC(c5c(C)cnn5C5(OC)CC5)CC4)nc(C(F)(F)F)n3)C2C)CC1C]

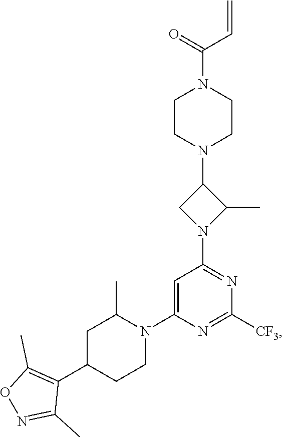 [SMILES: C=CC(=O)N1CCN(C2CN(c3cc(N4CCC(c5c(C)noc5C)CC4C)nc(C(F)(F)F)n3)C2C)CC1]

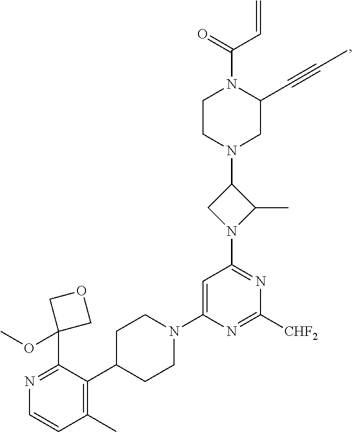 [SMILES: C=CC(=O)N1CCN(C2CN(c3cc(N4CCC(c5c(C)ccnc5C5(OC)COC5)CC4)nc(C(F)F)n3)C2C)CC1C#CC]

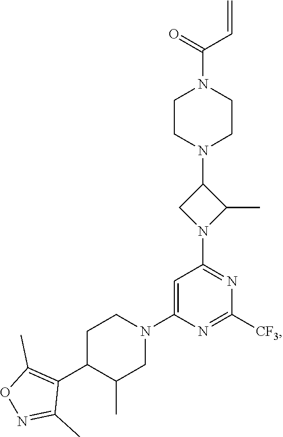 [SMILES: C=CC(=O)N1CCN(C2CN(c3cc(N4CCC(c5c(C)noc5C)C(C)C4)nc(C(F)(F)F)n3)C2C)CC1]

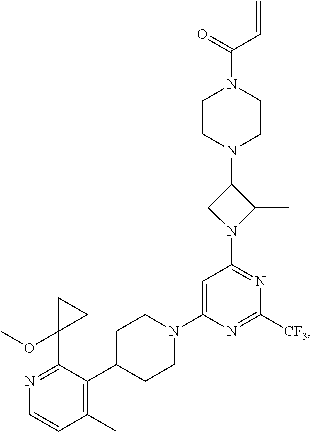 [SMILES: C=CC(=O)N1CCN(C2CN(c3cc(N4CCC(c5c(C)ccnc5C5(OC)CC5)CC4)nc(C(F)(F)F)n3)C2C)CC1]

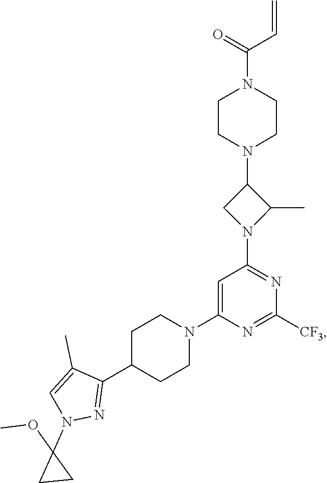 [SMILES: C=CC(=O)N1CCN(C2CN(c3cc(N4CCC(c5nn(C6(OC)CC6)cc5C)CC4)nc(C(F)(F)F)n3)C2C)CC1]

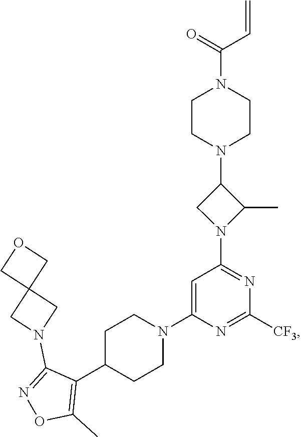 [SMILES: C=CC(=O)N1CCN(C2CN(c3cc(N4CCC(c5c(N6CC7(COC7)C6)noc5C)CC4)nc(C(F)(F)F)n3)C2C)CC1]

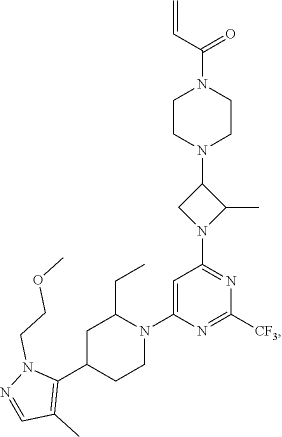 [SMILES: C=CC(=O)N1CCN(C2CN(c3cc(N4CCC(c5c(C)cnn5CCOC)CC4CC)nc(C(F)(F)F)n3)C2C)CC1]

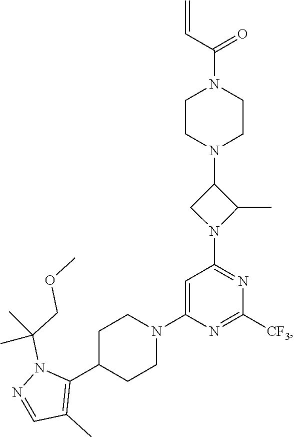 [SMILES: C=CC(=O)N1CCN(C2CN(c3cc(N4CCC(c5c(C)cnn5C(C)(C)COC)CC4)nc(C(F)(F)F)n3)C2C)CC1]

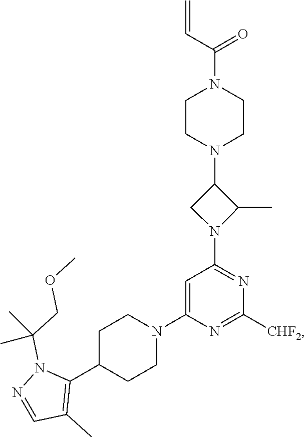 [SMILES: C=CC(=O)N1CCN(C2CN(c3cc(N4CCC(c5c(C)cnn5C(C)(C)COC)CC4)nc(C(F)F)n3)C2C)CC1]

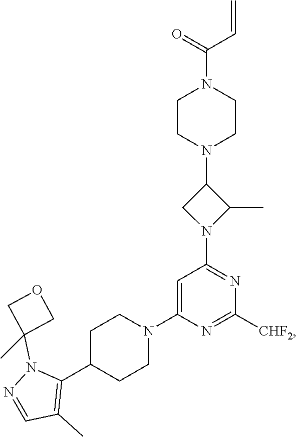 [SMILES: C=CC(=O)N1CCN(C2CN(c3cc(N4CCC(c5c(C)cnn5C5(C)COC5)CC4)nc(C(F)F)n3)C2C)CC1]

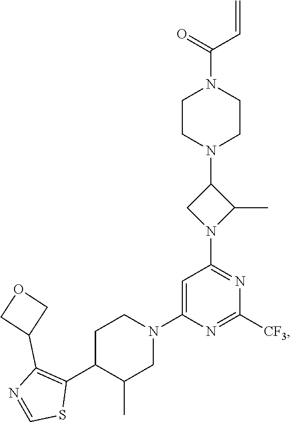 [SMILES: C=CC(=O)N1CCN(C2CN(c3cc(N4CCC(c5scnc5C5COC5)C(C)C4)nc(C(F)(F)F)n3)C2C)CC1]

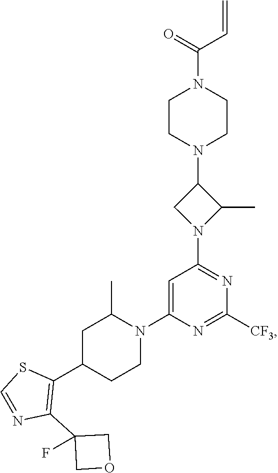 [SMILES: C=CC(=O)N1CCN(C2CN(c3cc(N4CCC(c5scnc5C5(F)COC5)CC4C)nc(C(F)(F)F)n3)C2C)CC1]

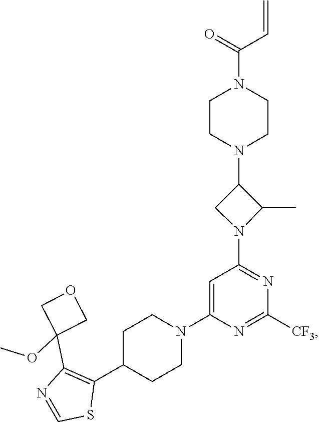 [SMILES: C=CC(=O)N1CCN(C2CN(c3cc(N4CCC(c5scnc5C5(OC)COC5)CC4)nc(C(F)(F)F)n3)C2C)CC1]

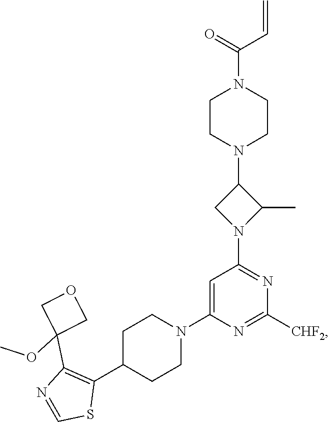 [SMILES: C=CC(=O)N1CCN(C2CN(c3cc(N4CCC(c5scnc5C5(OC)COC5)CC4)nc(C(F)F)n3)C2C)CC1]

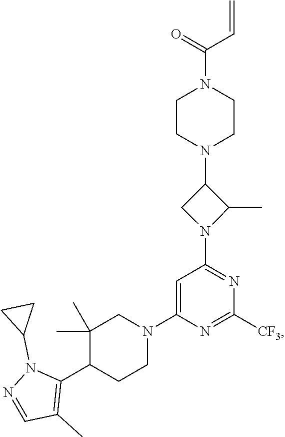 [SMILES: C=CC(=O)N1CCN(C2CN(c3cc(N4CCC(c5c(C)cnn5C5CC5)C(C)(C)C4)nc(C(F)(F)F)n3)C2C)CC1]

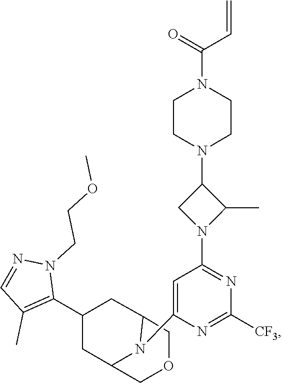 [SMILES: C=CC(=O)N1CCN(C2CN(c3cc(N4C5COCC4CC(c4c(C)cnn4CCOC)C5)nc(C(F)(F)F)n3)C2C)CC1]

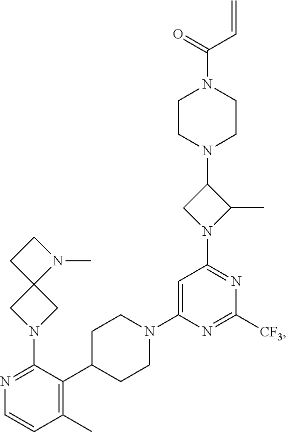 [SMILES: C=CC(=O)N1CCN(C2CN(c3cc(N4CCC(c5c(C)ccnc5N5CC6(CCN6C)C5)CC4)nc(C(F)(F)F)n3)C2C)CC1]

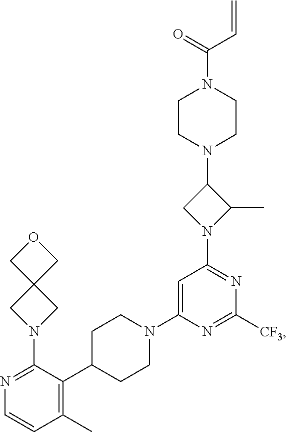 [SMILES: C=CC(=O)N1CCN(C2CN(c3cc(N4CCC(c5c(C)ccnc5N5CC6(COC6)C5)CC4)nc(C(F)(F)F)n3)C2C)CC1]

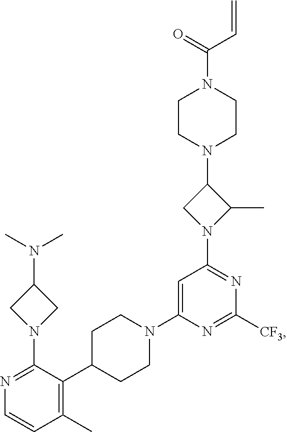 [SMILES: C=CC(=O)N1CCN(C2CN(c3cc(N4CCC(c5c(C)ccnc5N5CC(N(C)C)C5)CC4)nc(C(F)(F)F)n3)C2C)CC1]

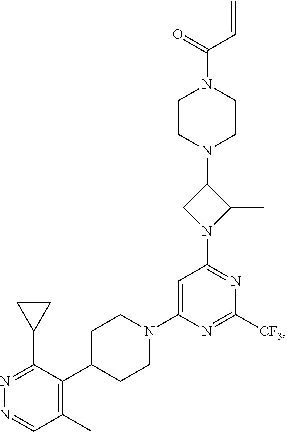 [SMILES: C=CC(=O)N1CCN(C2CN(c3cc(N4CCC(c5c(C)cnnc5C5CC5)CC4)nc(C(F)(F)F)n3)C2C)CC1]

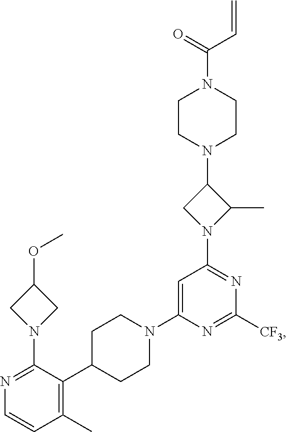 [SMILES: C=CC(=O)N1CCN(C2CN(c3cc(N4CCC(c5c(C)ccnc5N5CC(OC)C5)CC4)nc(C(F)(F)F)n3)C2C)CC1]

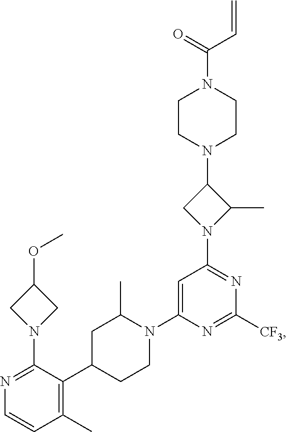 [SMILES: C=CC(=O)N1CCN(C2CN(c3cc(N4CCC(c5c(C)ccnc5N5CC(OC)C5)CC4C)nc(C(F)(F)F)n3)C2C)CC1]

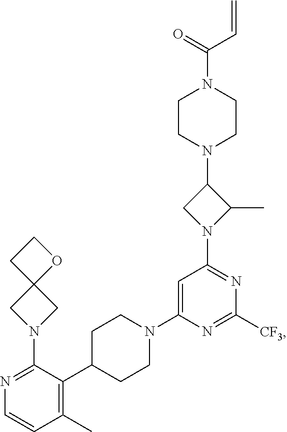 [SMILES: C=CC(=O)N1CCN(C2CN(c3cc(N4CCC(c5c(C)ccnc5N5CC6(CCO6)C5)CC4)nc(C(F)(F)F)n3)C2C)CC1]

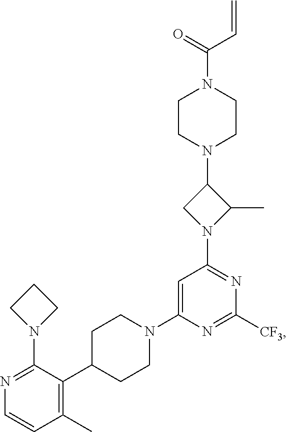 [SMILES: C=CC(=O)N1CCN(C2CN(c3cc(N4CCC(c5c(C)ccnc5N5CCC5)CC4)nc(C(F)(F)F)n3)C2C)CC1]

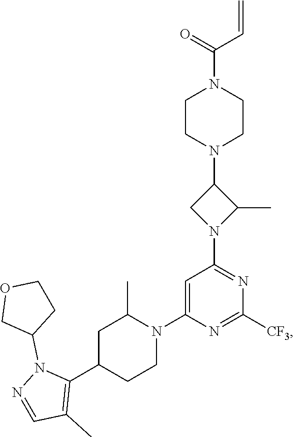 [SMILES: C=CC(=O)N1CCN(C2CN(c3cc(N4CCC(c5c(C)cnn5C5CCOC5)CC4C)nc(C(F)(F)F)n3)C2C)CC1]

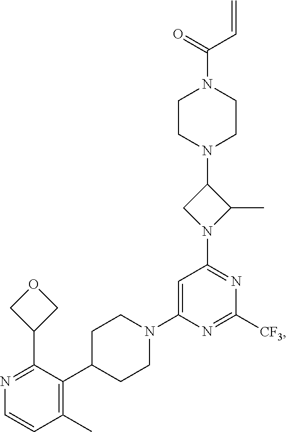 [SMILES: C=CC(=O)N1CCN(C2CN(c3cc(N4CCC(c5c(C)ccnc5C5COC5)CC4)nc(C(F)(F)F)n3)C2C)CC1]

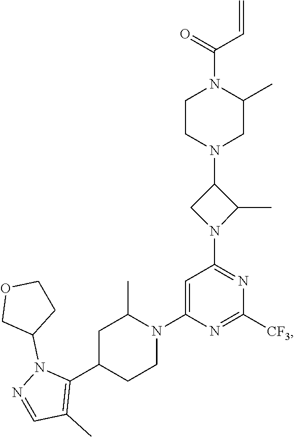 [SMILES: C=CC(=O)N1CCN(C2CN(c3cc(N4CCC(c5c(C)cnn5C5CCOC5)CC4C)nc(C(F)(F)F)n3)C2C)CC1C]

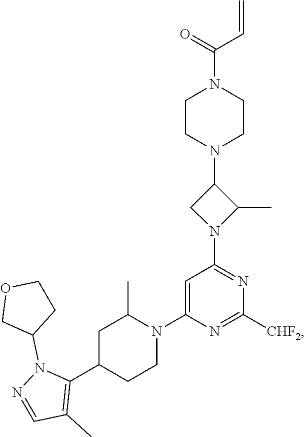 [SMILES: C=CC(=O)N1CCN(C2CN(c3cc(N4CCC(c5c(C)cnn5C5CCOC5)CC4C)nc(C(F)F)n3)C2C)CC1]

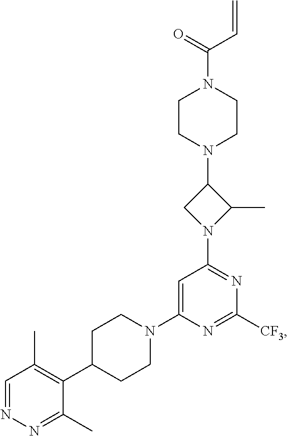 [SMILES: C=CC(=O)N1CCN(C2CN(c3cc(N4CCC(c5c(C)cnnc5C)CC4)nc(C(F)(F)F)n3)C2C)CC1]

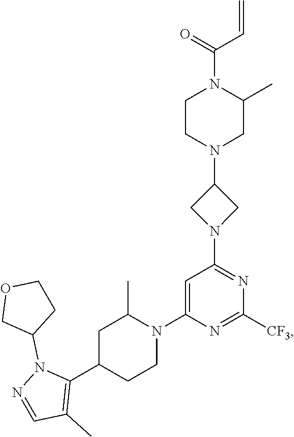 [SMILES: C=CC(=O)N1CCN(C2CN(c3cc(N4CCC(c5c(C)cnn5C5CCOC5)CC4C)nc(C(F)(F)F)n3)C2)CC1C]

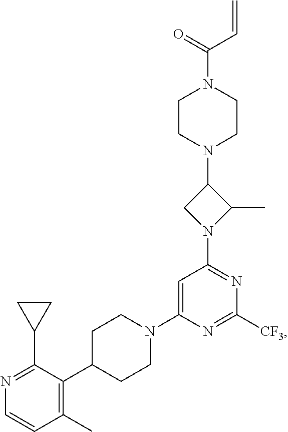 [SMILES: C=CC(=O)N1CCN(C2CN(c3cc(N4CCC(c5c(C)ccnc5C5CC5)CC4)nc(C(F)(F)F)n3)C2C)CC1]

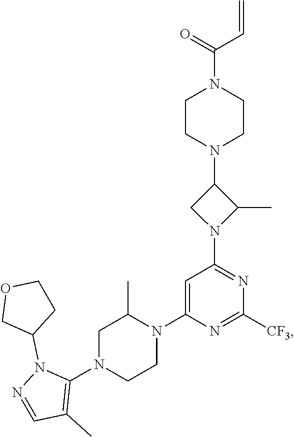 [SMILES: C=CC(=O)N1CCN(C2CN(c3cc(N4CCN(c5c(C)cnn5C5CCOC5)CC4C)nc(C(F)(F)F)n3)C2C)CC1]

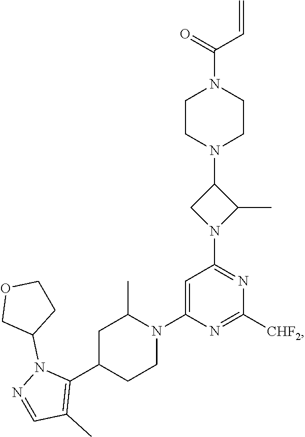 [SMILES: C=CC(=O)N1CCN(C2CN(c3cc(N4CCC(c5c(C)cnn5C5CCOC5)CC4C)nc(C(F)F)n3)C2C)CC1]

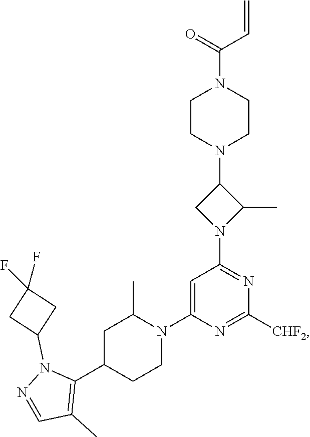 [SMILES: C=CC(=O)N1CCN(C2CN(c3cc(N4CCC(c5c(C)cnn5C5CC(F)(F)C5)CC4C)nc(C(F)F)n3)C2C)CC1]

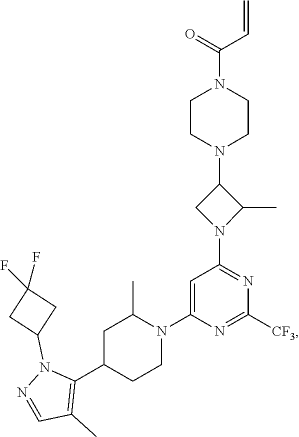 [SMILES: C=CC(=O)N1CCN(C2CN(c3cc(N4CCC(c5c(C)cnn5C5CC(F)(F)C5)CC4C)nc(C(F)(F)F)n3)C2C)CC1]

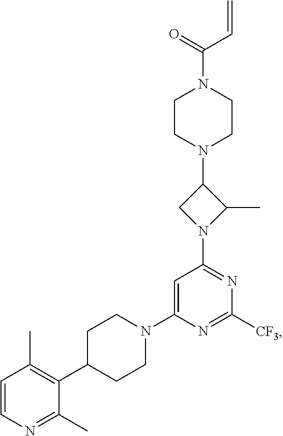 [SMILES: C=CC(=O)N1CCN(C2CN(c3cc(N4CCC(c5c(C)ccnc5C)CC4)nc(C(F)(F)F)n3)C2C)CC1]

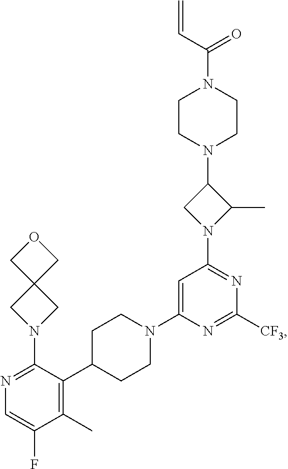 [SMILES: C=CC(=O)N1CCN(C2CN(c3cc(N4CCC(c5c(N6CC7(COC7)C6)ncc(F)c5C)CC4)nc(C(F)(F)F)n3)C2C)CC1]

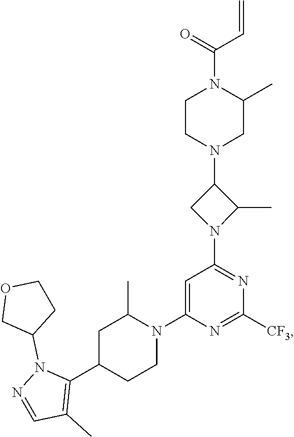 [SMILES: C=CC(=O)N1CCN(C2CN(c3cc(N4CCC(c5c(C)cnn5C5CCOC5)CC4C)nc(C(F)(F)F)n3)C2C)CC1C]

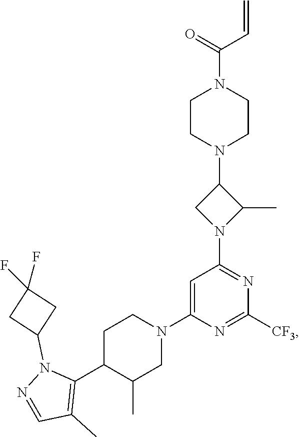 [SMILES: C=CC(=O)N1CCN(C2CN(c3cc(N4CCC(c5c(C)cnn5C5CC(F)(F)C5)C(C)C4)nc(C(F)(F)F)n3)C2C)CC1]

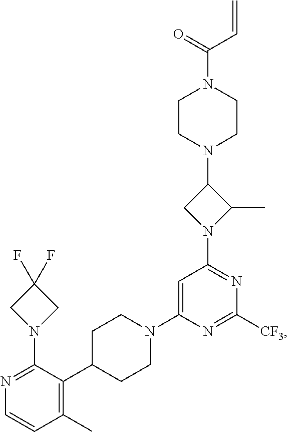 [SMILES: C=CC(=O)N1CCN(C2CN(c3cc(N4CCC(c5c(C)ccnc5N5CC(F)(F)C5)CC4)nc(C(F)(F)F)n3)C2C)CC1]

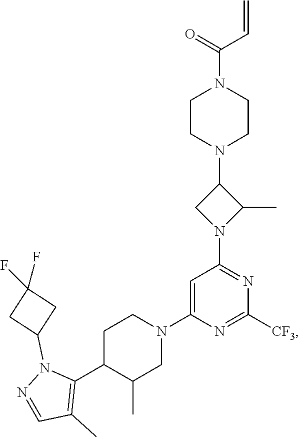 [SMILES: C=CC(=O)N1CCN(C2CN(c3cc(N4CCC(c5c(C)cnn5C5CC(F)(F)C5)C(C)C4)nc(C(F)(F)F)n3)C2C)CC1]

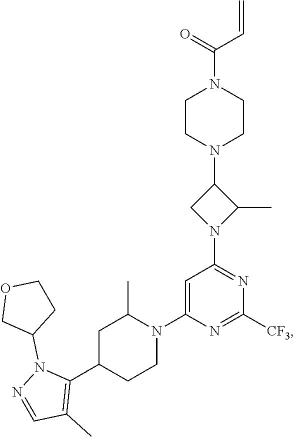 [SMILES: C=CC(=O)N1CCN(C2CN(c3cc(N4CCC(c5c(C)cnn5C5CCOC5)CC4C)nc(C(F)(F)F)n3)C2C)CC1]

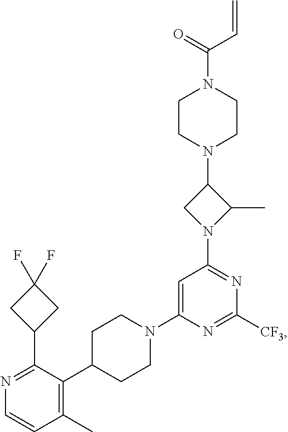 [SMILES: C=CC(=O)N1CCN(C2CN(c3cc(N4CCC(c5c(C)ccnc5C5CC(F)(F)C5)CC4)nc(C(F)(F)F)n3)C2C)CC1]

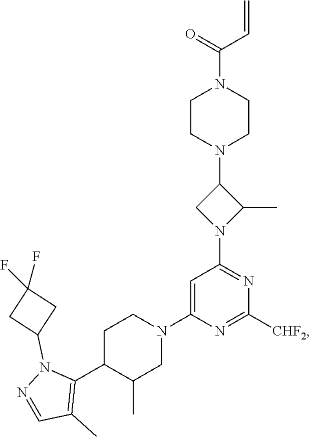 [SMILES: C=CC(=O)N1CCN(C2CN(c3cc(N4CCC(c5c(C)cnn5C5CC(F)(F)C5)C(C)C4)nc(C(F)F)n3)C2C)CC1]

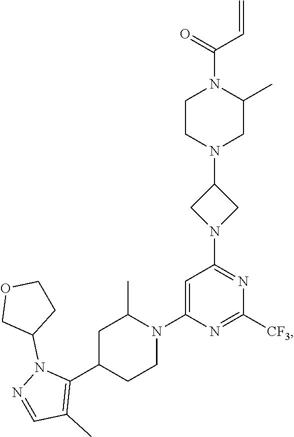 [SMILES: C=CC(=O)N1CCN(C2CN(c3cc(N4CCC(c5c(C)cnn5C5CCOC5)CC4C)nc(C(F)(F)F)n3)C2)CC1C]

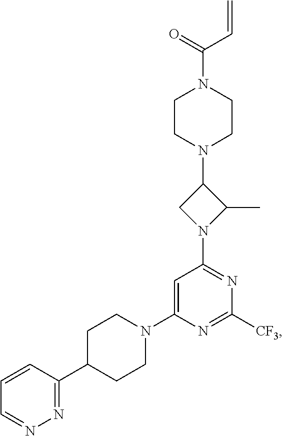 [SMILES: C=CC(=O)N1CCN(C2CN(c3cc(N4CCC(c5cccnn5)CC4)nc(C(F)(F)F)n3)C2C)CC1]

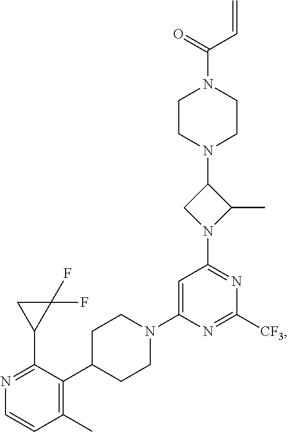 [SMILES: C=CC(=O)N1CCN(C2CN(c3cc(N4CCC(c5c(C)ccnc5C5CC5(F)F)CC4)nc(C(F)(F)F)n3)C2C)CC1]

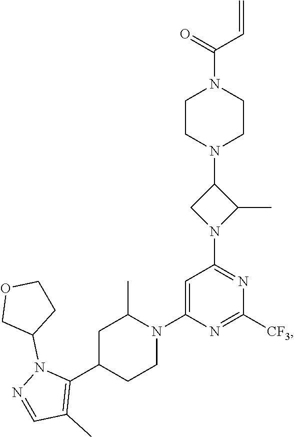 [SMILES: C=CC(=O)N1CCN(C2CN(c3cc(N4CCC(c5c(C)cnn5C5CCOC5)CC4C)nc(C(F)(F)F)n3)C2C)CC1]

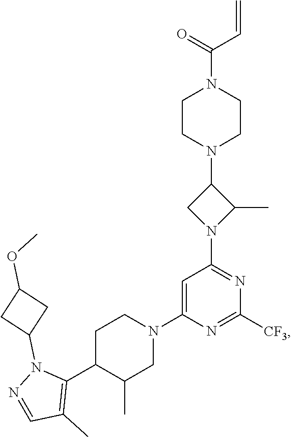 [SMILES: C=CC(=O)N1CCN(C2CN(c3cc(N4CCC(c5c(C)cnn5C5CC(OC)C5)C(C)C4)nc(C(F)(F)F)n3)C2C)CC1]

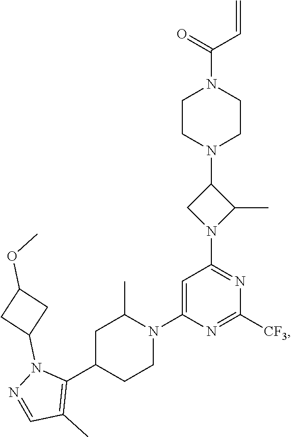 [SMILES: C=CC(=O)N1CCN(C2CN(c3cc(N4CCC(c5c(C)cnn5C5CC(OC)C5)CC4C)nc(C(F)(F)F)n3)C2C)CC1]

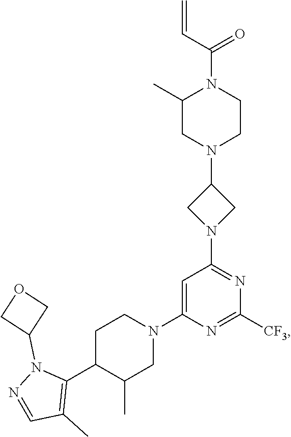 [SMILES: C=CC(=O)N1CCN(C2CN(c3cc(N4CCC(c5c(C)cnn5C5COC5)C(C)C4)nc(C(F)(F)F)n3)C2)CC1C]

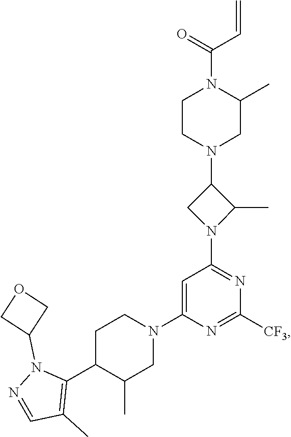 [SMILES: C=CC(=O)N1CCN(C2CN(c3cc(N4CCC(c5c(C)cnn5C5COC5)C(C)C4)nc(C(F)(F)F)n3)C2C)CC1C]

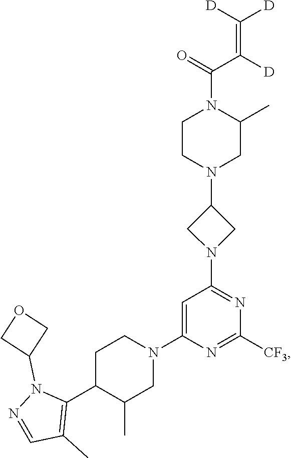 [SMILES: [2H]C([2H])=C([2H])C(=O)N1CCN(C2CN(c3cc(N4CCC(c5c(C)cnn5C5COC5)C(C)C4)nc(C(F)(F)F)n3)C2)CC1C]

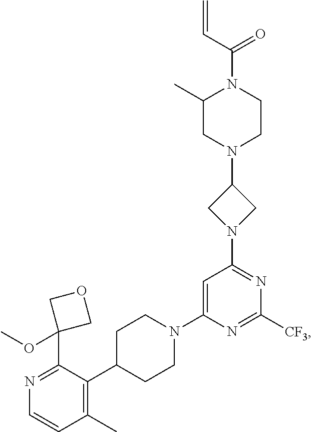 [SMILES: C=CC(=O)N1CCN(C2CN(c3cc(N4CCC(c5c(C)ccnc5C5(OC)COC5)CC4)nc(C(F)(F)F)n3)C2)CC1C]

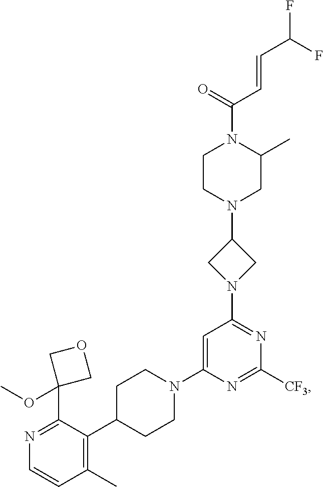 [SMILES: COC1(c2nccc(C)c2C2CCN(c3cc(N4CC(N5CCN(C(=O)/C=C/C(F)F)C(C)C5)C4)nc(C(F)(F)F)n3)CC2)COC1]

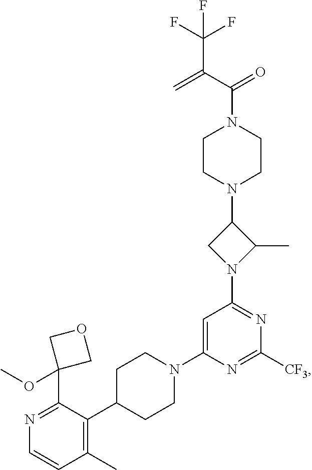 [SMILES: C=C(C(=O)N1CCN(C2CN(c3cc(N4CCC(c5c(C)ccnc5C5(OC)COC5)CC4)nc(C(F)(F)F)n3)C2C)CC1)C(F)(F)F]

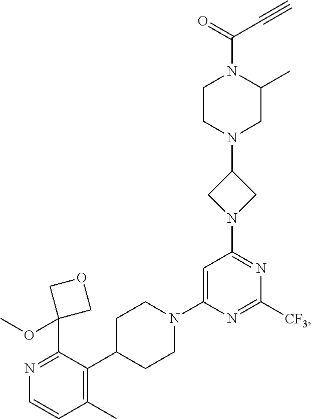 [SMILES: C#CC(=O)N1CCN(C2CN(c3cc(N4CCC(c5c(C)ccnc5C5(OC)COC5)CC4)nc(C(F)(F)F)n3)C2)CC1C]

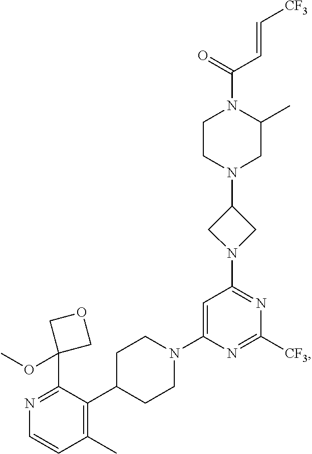 [SMILES: COC1(c2nccc(C)c2C2CCN(c3cc(N4CC(N5CCN(C(=O)/C=C/C(F)(F)F)C(C)C5)C4)nc(C(F)(F)F)n3)CC2)COC1]

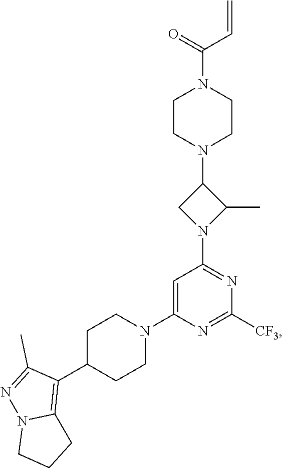 [SMILES: C=CC(=O)N1CCN(C2CN(c3cc(N4CCC(c5c(C)nn6c5CCC6)CC4)nc(C(F)(F)F)n3)C2C)CC1]

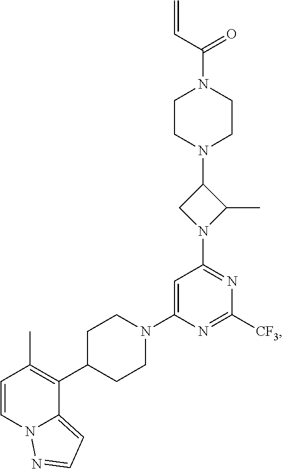 [SMILES: C=CC(=O)N1CCN(C2CN(c3cc(N4CCC(c5c(C)ccn6nccc56)CC4)nc(C(F)(F)F)n3)C2C)CC1]

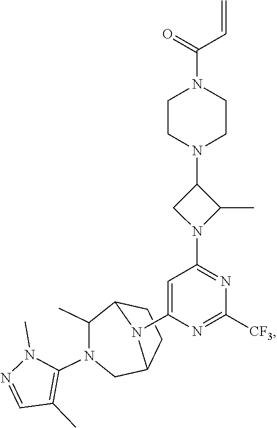 [SMILES: C=CC(=O)N1CCN(C2CN(c3cc(N4C5CCC4C(C)N(c4c(C)cnn4C)C5)nc(C(F)(F)F)n3)C2C)CC1]

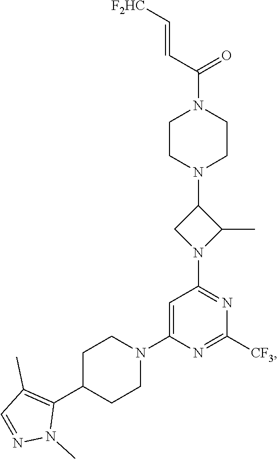 [SMILES: Cc1cnn(C)c1C1CCN(c2cc(N3CC(N4CCN(C(=O)/C=C/C(F)F)CC4)C3C)nc(C(F)(F)F)n2)CC1]

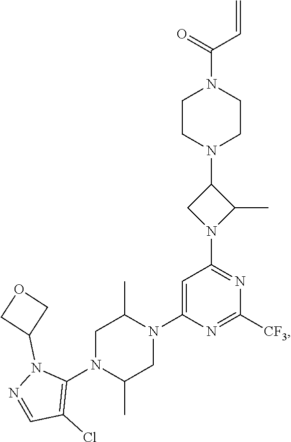 [SMILES: C=CC(=O)N1CCN(C2CN(c3cc(N4CC(C)N(c5c(Cl)cnn5C5COC5)CC4C)nc(C(F)(F)F)n3)C2C)CC1]

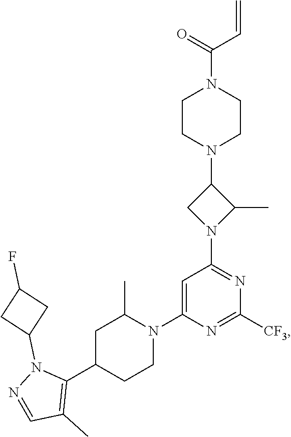 [SMILES: C=CC(=O)N1CCN(C2CN(c3cc(N4CCC(c5c(C)cnn5C5CC(F)C5)CC4C)nc(C(F)(F)F)n3)C2C)CC1]

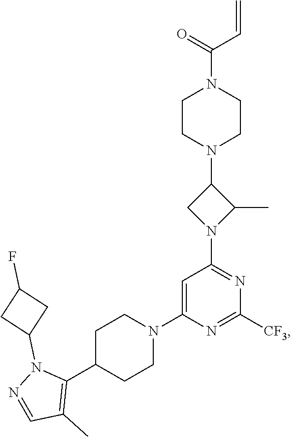 [SMILES: C=CC(=O)N1CCN(C2CN(c3cc(N4CCC(c5c(C)cnn5C5CC(F)C5)CC4)nc(C(F)(F)F)n3)C2C)CC1]

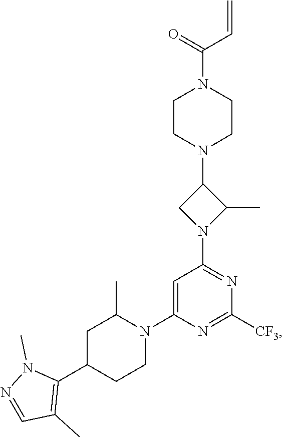 [SMILES: C=CC(=O)N1CCN(C2CN(c3cc(N4CCC(c5c(C)cnn5C)CC4C)nc(C(F)(F)F)n3)C2C)CC1]

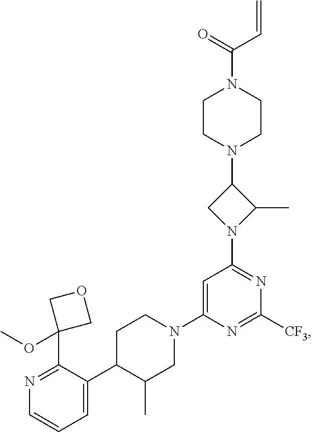 [SMILES: C=CC(=O)N1CCN(C2CN(c3cc(N4CCC(c5cccnc5C5(OC)COC5)C(C)C4)nc(C(F)(F)F)n3)C2C)CC1]

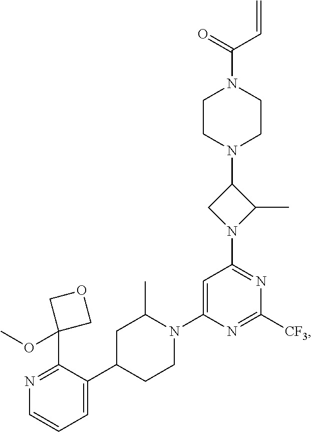 [SMILES: C=CC(=O)N1CCN(C2CN(c3cc(N4CCC(c5cccnc5C5(OC)COC5)CC4C)nc(C(F)(F)F)n3)C2C)CC1]

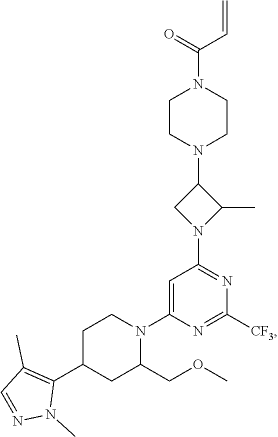 [SMILES: C=CC(=O)N1CCN(C2CN(c3cc(N4CCC(c5c(C)cnn5C)CC4COC)nc(C(F)(F)F)n3)C2C)CC1]

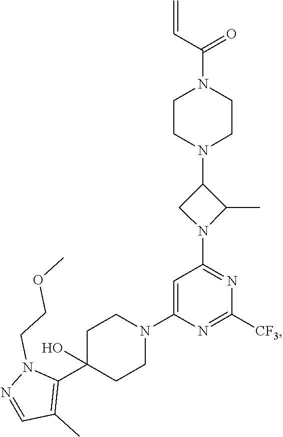 [SMILES: C=CC(=O)N1CCN(C2CN(c3cc(N4CCC(O)(c5c(C)cnn5CCOC)CC4)nc(C(F)(F)F)n3)C2C)CC1]

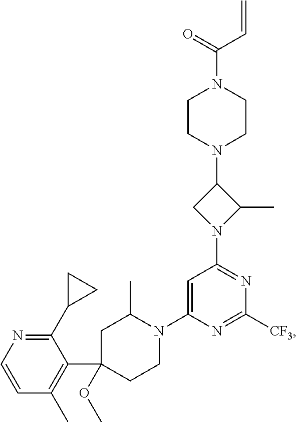 [SMILES: C=CC(=O)N1CCN(C2CN(c3cc(N4CCC(OC)(c5c(C)ccnc5C5CC5)CC4C)nc(C(F)(F)F)n3)C2C)CC1]

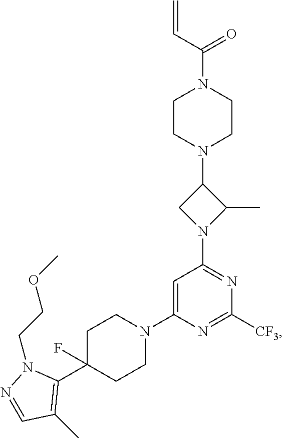 [SMILES: C=CC(=O)N1CCN(C2CN(c3cc(N4CCC(F)(c5c(C)cnn5CCOC)CC4)nc(C(F)(F)F)n3)C2C)CC1]

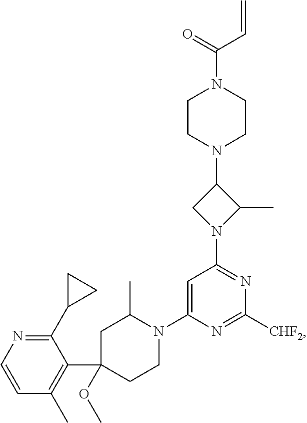 [SMILES: C=CC(=O)N1CCN(C2CN(c3cc(N4CCC(OC)(c5c(C)ccnc5C5CC5)CC4C)nc(C(F)F)n3)C2C)CC1]

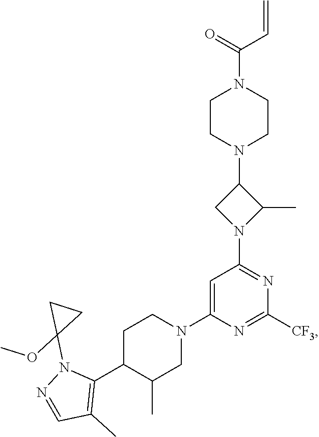 [SMILES: C=CC(=O)N1CCN(C2CN(c3cc(N4CCC(c5c(C)cnn5C5(OC)CC5)C(C)C4)nc(C(F)(F)F)n3)C2C)CC1]

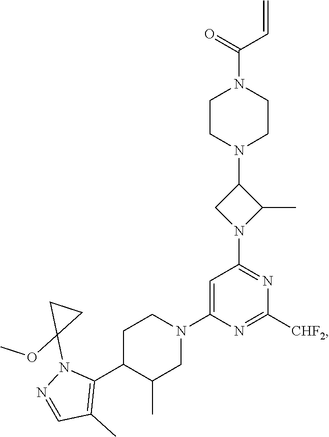 [SMILES: C=CC(=O)N1CCN(C2CN(c3cc(N4CCC(c5c(C)cnn5C5(OC)CC5)C(C)C4)nc(C(F)F)n3)C2C)CC1]

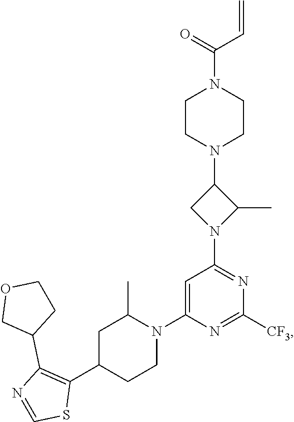 [SMILES: C=CC(=O)N1CCN(C2CN(c3cc(N4CCC(c5scnc5C5CCOC5)CC4C)nc(C(F)(F)F)n3)C2C)CC1]

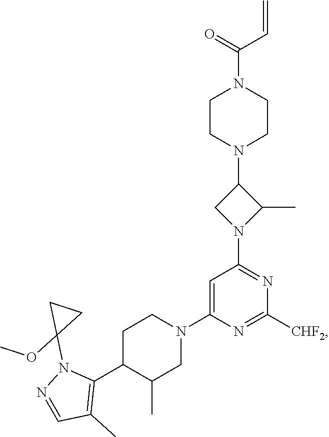 [SMILES: C=CC(=O)N1CCN(C2CN(c3cc(N4CCC(c5c(C)cnn5C5(OC)CC5)C(C)C4)nc(C(F)F)n3)C2C)CC1]

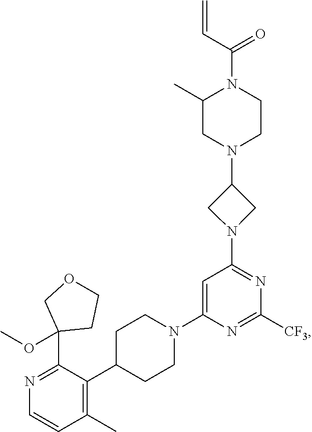

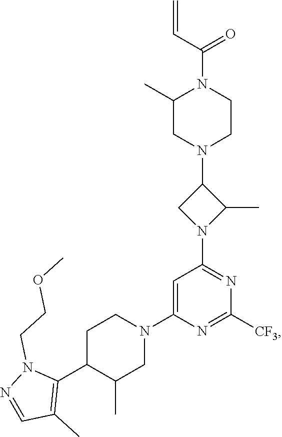 [SMILES: C=CC(=O)N1CCN(C2CN(c3cc(N4CCC(c5c(C)cnn5CCOC)C(C)C4)nc(C(F)(F)F)n3)C2C)CC1C]

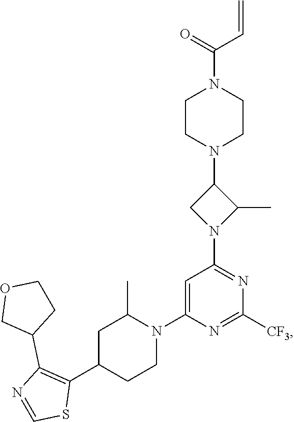 [SMILES: C=CC(=O)N1CCN(C2CN(c3cc(N4CCC(c5scnc5C5CCOC5)CC4C)nc(C(F)(F)F)n3)C2C)CC1]

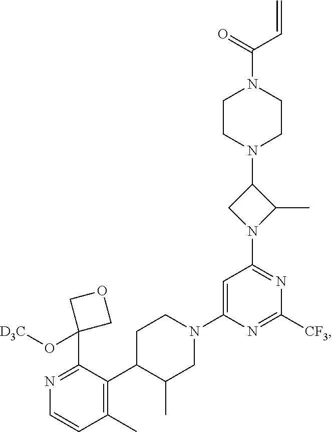 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2C)COC1]

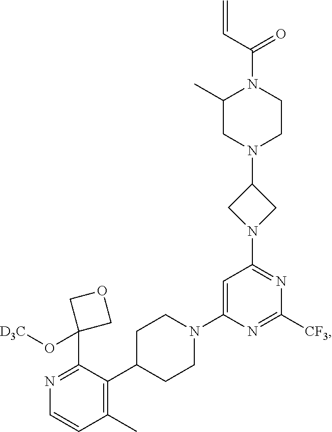 [SMILES: [2H]C([2H])([2H])OC1(c2nccc(C)c2C2CCN(c3cc(N4CC(N5CCN(C(=O)C=C)C(C)C5)C4)nc(C(F)(F)F)n3)CC2)COC1]

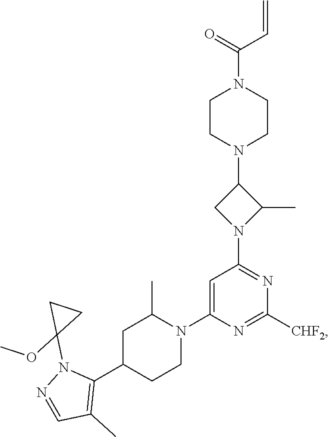 [SMILES: C=CC(=O)N1CCN(C2CN(c3cc(N4CCC(c5c(C)cnn5C5(OC)CC5)CC4C)nc(C(F)F)n3)C2C)CC1]

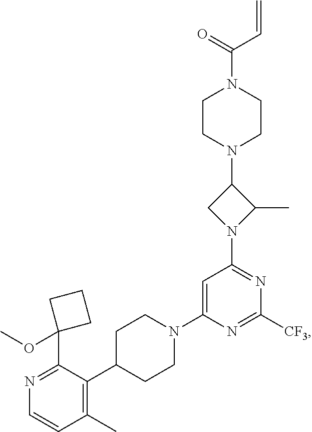 [SMILES: C=CC(=O)N1CCN(C2CN(c3cc(N4CCC(c5c(C)ccnc5C5(OC)CCC5)CC4)nc(C(F)(F)F)n3)C2C)CC1]

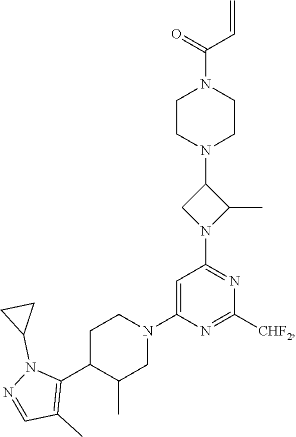 [SMILES: C=CC(=O)N1CCN(C2CN(c3cc(N4CCC(c5c(C)cnn5C5CC5)C(C)C4)nc(C(F)F)n3)C2C)CC1]

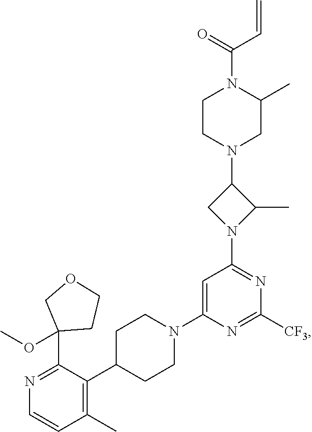 [SMILES: C=CC(=O)N1CCN(C2CN(c3cc(N4CCC(c5c(C)ccnc5C5(OC)CCOC5)CC4)nc(C(F)(F)F)n3)C2C)CC1C]

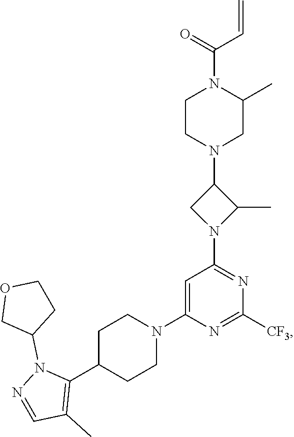 [SMILES: C=CC(=O)N1CCN(C2CN(c3cc(N4CCC(c5c(C)cnn5C5CCOC5)CC4)nc(C(F)(F)F)n3)C2C)CC1C]

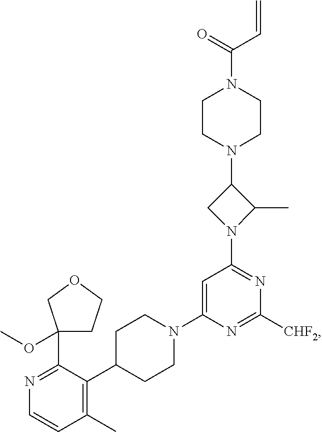 [SMILES: C=CC(=O)N1CCN(C2CN(c3cc(N4CCC(c5c(C)ccnc5C5(OC)CCOC5)CC4)nc(C(F)F)n3)C2C)CC1]

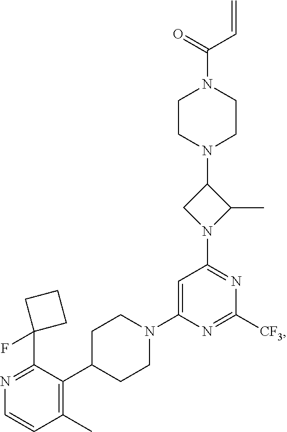 [SMILES: C=CC(=O)N1CCN(C2CN(c3cc(N4CCC(c5c(C)ccnc5C5(F)CCC5)CC4)nc(C(F)(F)F)n3)C2C)CC1]

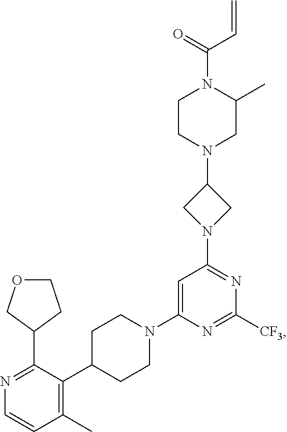 [SMILES: C=CC(=O)N1CCN(C2CN(c3cc(N4CCC(c5c(C)ccnc5C5CCOC5)CC4)nc(C(F)(F)F)n3)C2)CC1C]

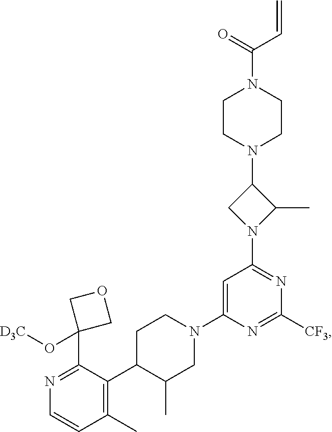 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2C)COC1]

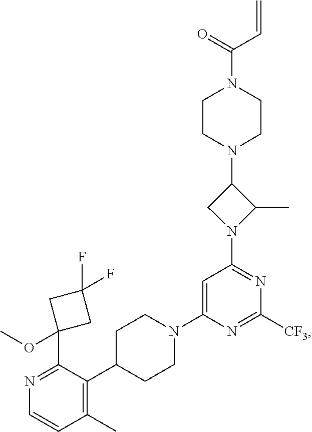 [SMILES: C=CC(=O)N1CCN(C2CN(c3cc(N4CCC(c5c(C)ccnc5C5(OC)CC(F)(F)C5)CC4)nc(C(F)(F)F)n3)C2C)CC1]

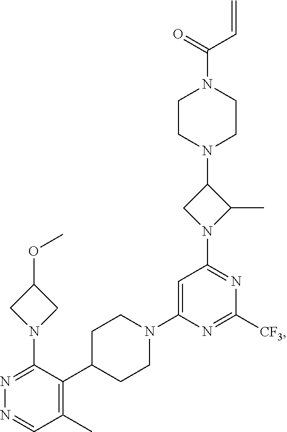 [SMILES: C=CC(=O)N1CCN(C2CN(c3cc(N4CCC(c5c(C)cnnc5N5CC(OC)C5)CC4)nc(C(F)(F)F)n3)C2C)CC1]

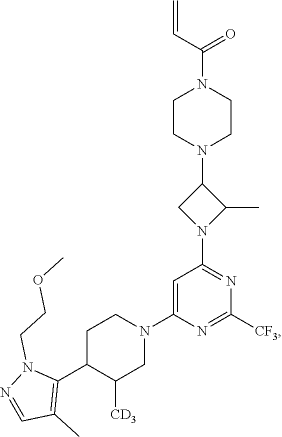 [SMILES: [2H]C([2H])([2H])C1CN(c2cc(N3CC(N4CCN(C(=O)C=C)CC4)C3C)nc(C(F)(F)F)n2)CCC1c1c(C)cnn1CCOC]

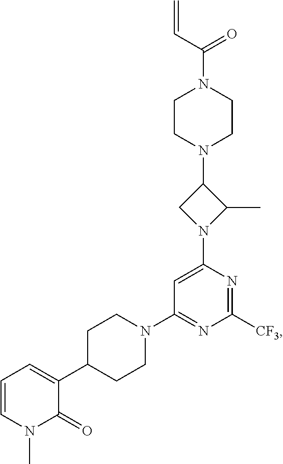 [SMILES: C=CC(=O)N1CCN(C2CN(c3cc(N4CCC(c5cccn(C)c5=O)CC4)nc(C(F)(F)F)n3)C2C)CC1]

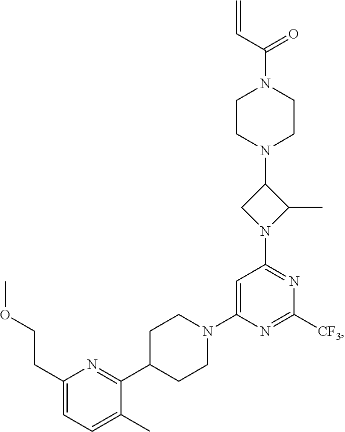 [SMILES: C=CC(=O)N1CCN(C2CN(c3cc(N4CCC(c5nc(CCOC)ccc5C)CC4)nc(C(F)(F)F)n3)C2C)CC1]

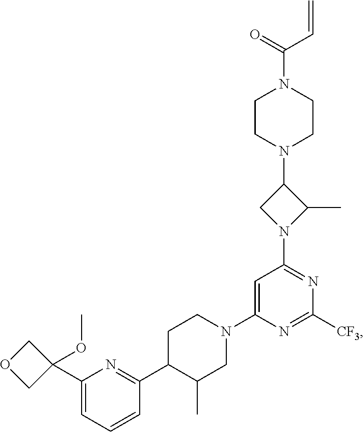 [SMILES: C=CC(=O)N1CCN(C2CN(c3cc(N4CCC(c5cccc(C6(OC)COC6)n5)C(C)C4)nc(C(F)(F)F)n3)C2C)CC1]

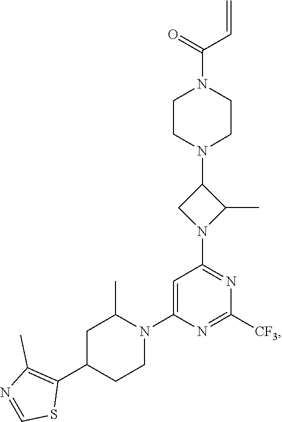 [SMILES: C=CC(=O)N1CCN(C2CN(c3cc(N4CCC(c5scnc5C)CC4C)nc(C(F)(F)F)n3)C2C)CC1]

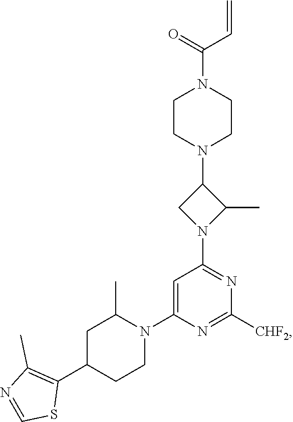 [SMILES: C=CC(=O)N1CCN(C2CN(c3cc(N4CCC(c5scnc5C)CC4C)nc(C(F)F)n3)C2C)CC1]

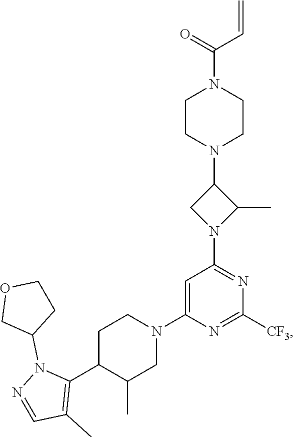 [SMILES: C=CC(=O)N1CCN(C2CN(c3cc(N4CCC(c5c(C)cnn5C5CCOC5)C(C)C4)nc(C(F)(F)F)n3)C2C)CC1]

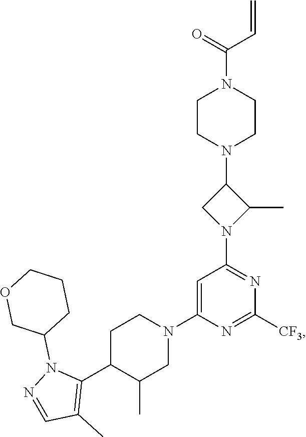 [SMILES: C=CC(=O)N1CCN(C2CN(c3cc(N4CCC(c5c(C)cnn5C5CCCOC5)C(C)C4)nc(C(F)(F)F)n3)C2C)CC1]

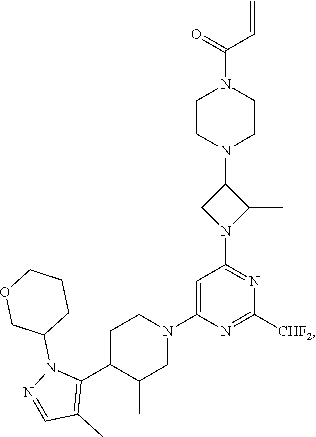 [SMILES: C=CC(=O)N1CCN(C2CN(c3cc(N4CCC(c5c(C)cnn5C5CCCOC5)C(C)C4)nc(C(F)F)n3)C2C)CC1]

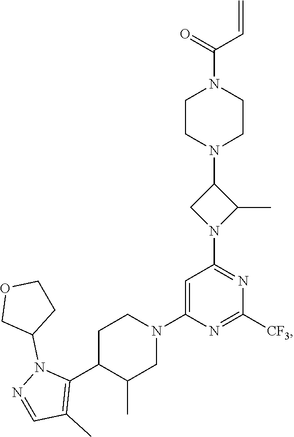 [SMILES: C=CC(=O)N1CCN(C2CN(c3cc(N4CCC(c5c(C)cnn5C5CCOC5)C(C)C4)nc(C(F)(F)F)n3)C2C)CC1]

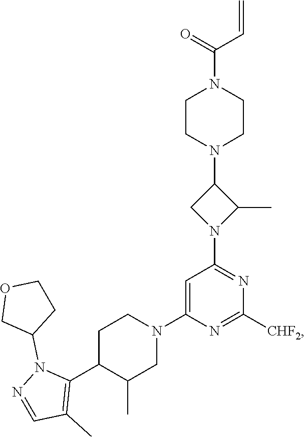 [SMILES: C=CC(=O)N1CCN(C2CN(c3cc(N4CCC(c5c(C)cnn5C5CCOC5)C(C)C4)nc(C(F)F)n3)C2C)CC1]

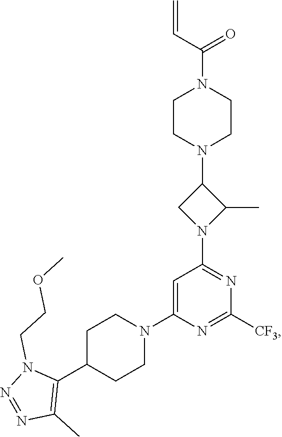 [SMILES: C=CC(=O)N1CCN(C2CN(c3cc(N4CCC(c5c(C)nnn5CCOC)CC4)nc(C(F)(F)F)n3)C2C)CC1]

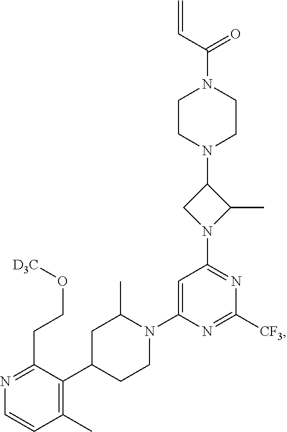 [SMILES: [2H]C([2H])([2H])OCCc1nccc(C)c1C1CCN(c2cc(N3CC(N4CCN(C(=O)C=C)CC4)C3C)nc(C(F)(F)F)n2)C(C)C1]

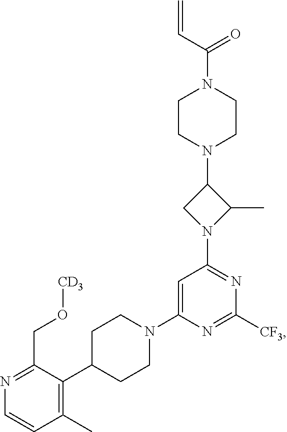 [SMILES: [2H]C([2H])([2H])OCc1nccc(C)c1C1CCN(c2cc(N3CC(N4CCN(C(=O)C=C)CC4)C3C)nc(C(F)(F)F)n2)CC1]

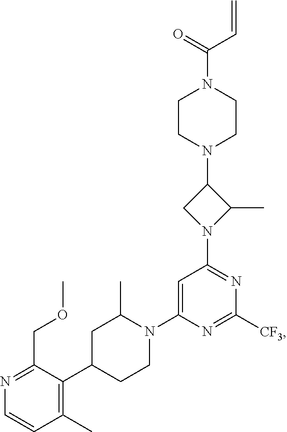 [SMILES: C=CC(=O)N1CCN(C2CN(c3cc(N4CCC(c5c(C)ccnc5COC)CC4C)nc(C(F)(F)F)n3)C2C)CC1]

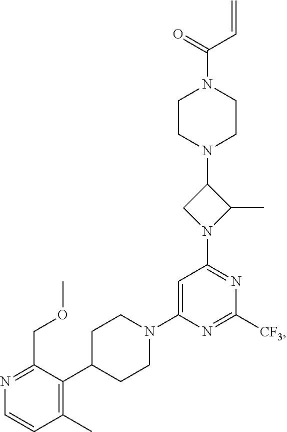 [SMILES: C=CC(=O)N1CCN(C2CN(c3cc(N4CCC(c5c(C)ccnc5COC)CC4)nc(C(F)(F)F)n3)C2C)CC1]

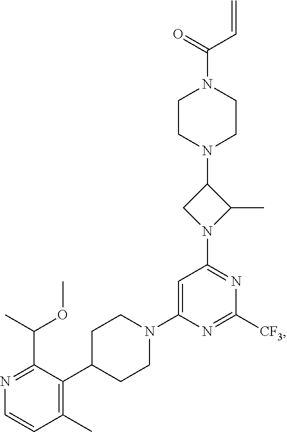 [SMILES: C=CC(=O)N1CCN(C2CN(c3cc(N4CCC(c5c(C)ccnc5C(C)OC)CC4)nc(C(F)(F)F)n3)C2C)CC1]

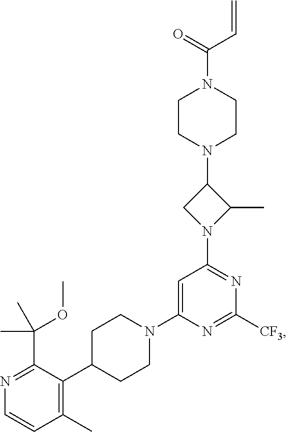 [SMILES: C=CC(=O)N1CCN(C2CN(c3cc(N4CCC(c5c(C)ccnc5C(C)(C)OC)CC4)nc(C(F)(F)F)n3)C2C)CC1]

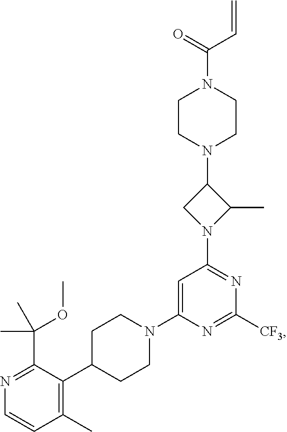 [SMILES: C=CC(=O)N1CCN(C2CN(c3cc(N4CCC(c5c(C)ccnc5C(C)(C)OC)CC4)nc(C(F)(F)F)n3)C2C)CC1]

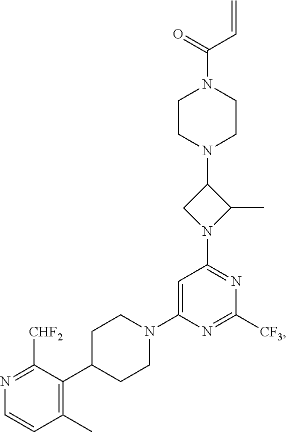 [SMILES: C=CC(=O)N1CCN(C2CN(c3cc(N4CCC(c5c(C)ccnc5C(F)F)CC4)nc(C(F)(F)F)n3)C2C)CC1]

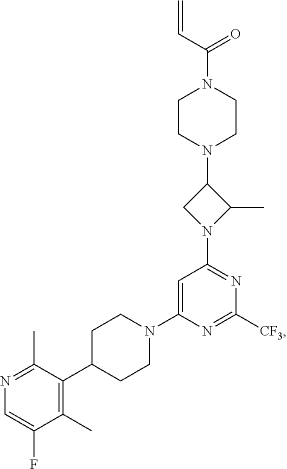 [SMILES: C=CC(=O)N1CCN(C2CN(c3cc(N4CCC(c5c(C)ncc(F)c5C)CC4)nc(C(F)(F)F)n3)C2C)CC1]

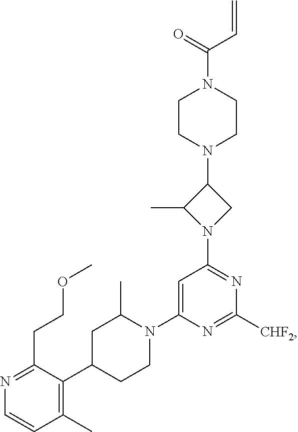 [SMILES: C=CC(=O)N1CCN(C2CN(c3cc(N4CCC(c5c(C)ccnc5CCOC)CC4C)nc(C(F)F)n3)C2C)CC1]

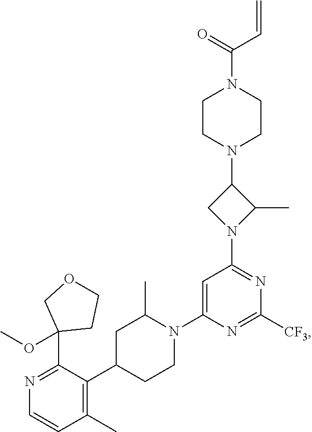 [SMILES: C=CC(=O)N1CCN(C2CN(c3cc(N4CCC(c5c(C)ccnc5C5(OC)CCOC5)CC4C)nc(C(F)(F)F)n3)C2C)CC1]

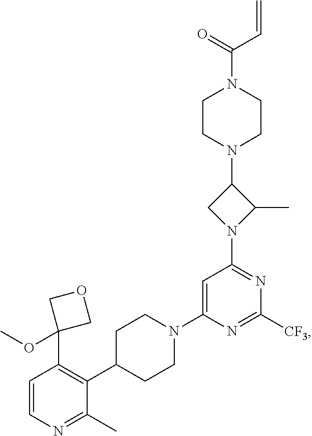 [SMILES: C=CC(=O)N1CCN(C2CN(c3cc(N4CCC(c5c(C6(OC)COC6)ccnc5C)CC4)nc(C(F)(F)F)n3)C2C)CC1]

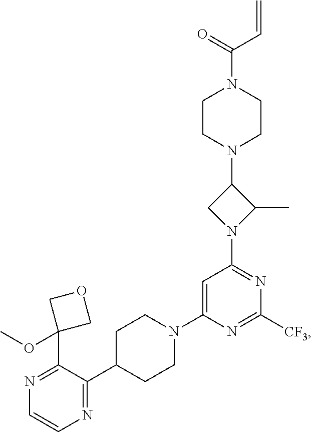 [SMILES: C=CC(=O)N1CCN(C2CN(c3cc(N4CCC(c5nccnc5C5(OC)COC5)CC4)nc(C(F)(F)F)n3)C2C)CC1]

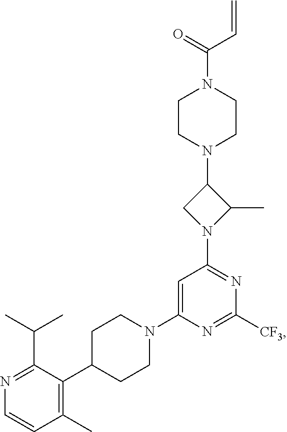 [SMILES: C=CC(=O)N1CCN(C2CN(c3cc(N4CCC(c5c(C)ccnc5C(C)C)CC4)nc(C(F)(F)F)n3)C2C)CC1]

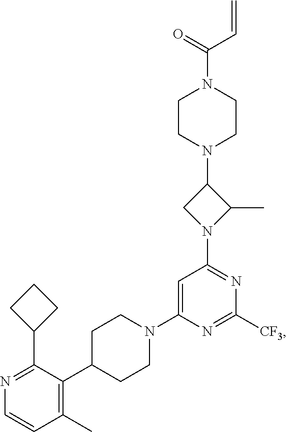 [SMILES: C=CC(=O)N1CCN(C2CN(c3cc(N4CCC(c5c(C)ccnc5C5CCC5)CC4)nc(C(F)(F)F)n3)C2C)CC1]

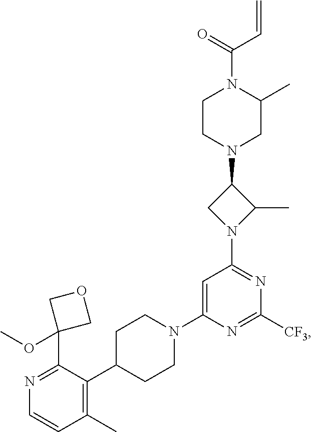 [SMILES: C=CC(=O)N1CCN([C@@H]2CN(c3cc(N4CCC(c5c(C)ccnc5C5(OC)COC5)CC4)nc(C(F)(F)F)n3)C2C)CC1C]

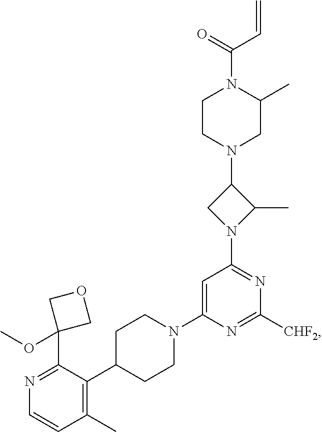 [SMILES: C=CC(=O)N1CCN(C2CN(c3cc(N4CCC(c5c(C)ccnc5C5(OC)COC5)CC4)nc(C(F)F)n3)C2C)CC1C]

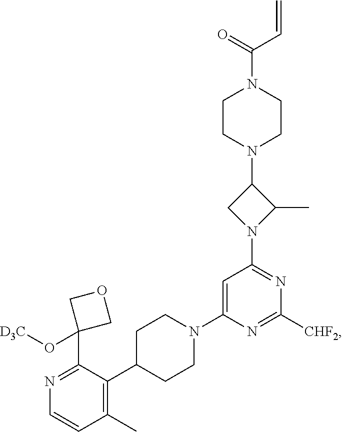 [SMILES: [2H]C([2H])([2H])OC1(c2nccc(C)c2C2CCN(c3cc(N4CC(N5CCN(C(=O)C=C)CC5)C4C)nc(C(F)F)n3)CC2)COC1]

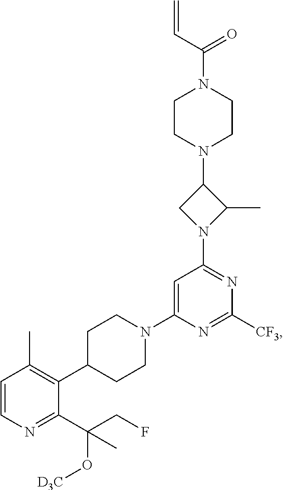 [SMILES: [2H]C([2H])([2H])OC(C)(CF)c1nccc(C)c1C1CCN(c2cc(N3CC(N4CCN(C(=O)C=C)CC4)C3C)nc(C(F)(F)F)n2)CC1]

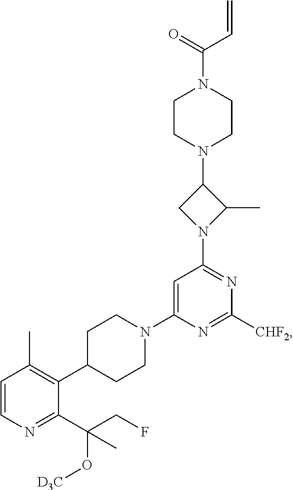 [SMILES: [2H]C([2H])([2H])OC(C)(CF)c1nccc(C)c1C1CCN(c2cc(N3CC(N4CCN(C(=O)C=C)CC4)C3C)nc(C(F)F)n2)CC1]

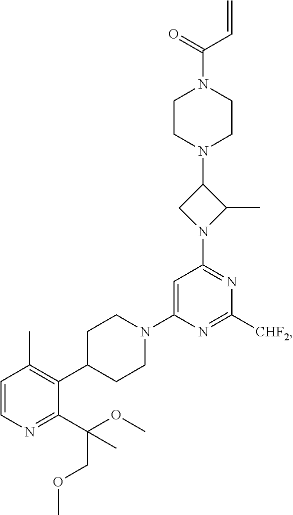 [SMILES: C=CC(=O)N1CCN(C2CN(c3cc(N4CCC(c5c(C)ccnc5C(C)(COC)OC)CC4)nc(C(F)F)n3)C2C)CC1]

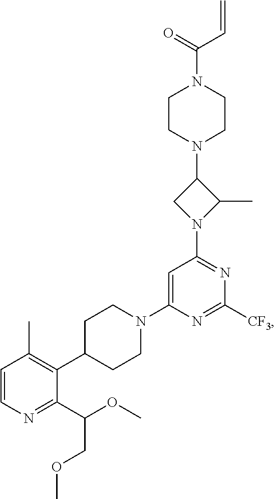 [SMILES: C=CC(=O)N1CCN(C2CN(c3cc(N4CCC(c5c(C)ccnc5C(COC)OC)CC4)nc(C(F)(F)F)n3)C2C)CC1]

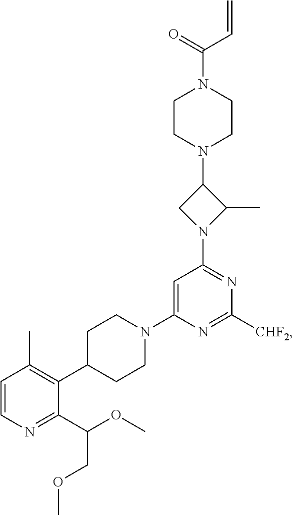 [SMILES: C=CC(=O)N1CCN(C2CN(c3cc(N4CCC(c5c(C)ccnc5C(COC)OC)CC4)nc(C(F)F)n3)C2C)CC1]

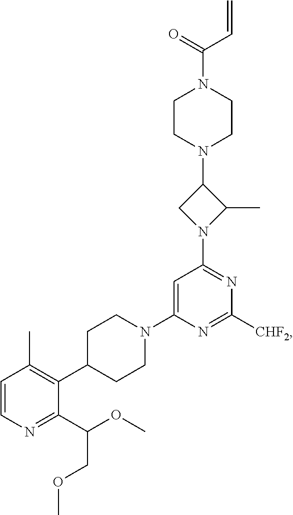 [SMILES: C=CC(=O)N1CCN(C2CN(c3cc(N4CCC(c5c(C)ccnc5C(COC)OC)CC4)nc(C(F)F)n3)C2C)CC1]

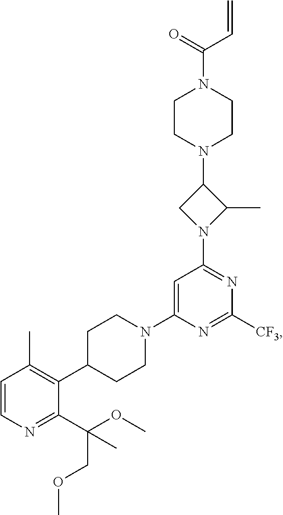 [SMILES: C=CC(=O)N1CCN(C2CN(c3cc(N4CCC(c5c(C)ccnc5C(C)(COC)OC)CC4)nc(C(F)(F)F)n3)C2C)CC1]

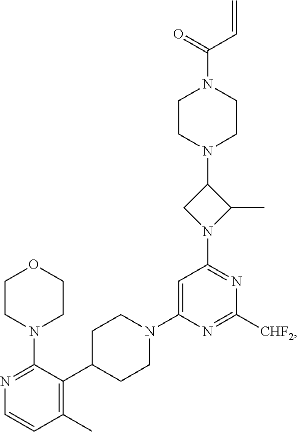 [SMILES: C=CC(=O)N1CCN(C2CN(c3cc(N4CCC(c5c(C)ccnc5N5CCOCC5)CC4)nc(C(F)F)n3)C2C)CC1]

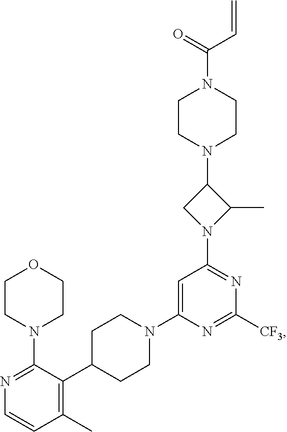 [SMILES: C=CC(=O)N1CCN(C2CN(c3cc(N4CCC(c5c(C)ccnc5N5CCOCC5)CC4)nc(C(F)(F)F)n3)C2C)CC1]

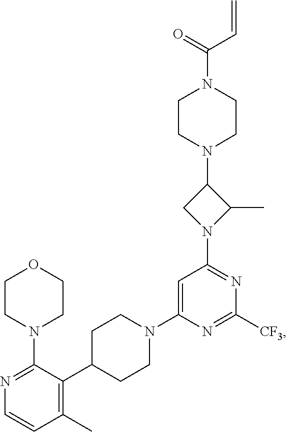 [SMILES: C=CC(=O)N1CCN(C2CN(c3cc(N4CCC(c5c(C)ccnc5N5CCOCC5)CC4)nc(C(F)(F)F)n3)C2C)CC1]

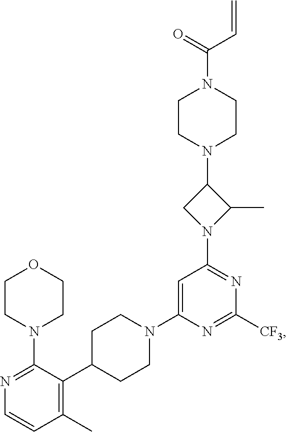 [SMILES: C=CC(=O)N1CCN(C2CN(c3cc(N4CCC(c5c(C)ccnc5N5CCOCC5)CC4)nc(C(F)(F)F)n3)C2C)CC1]

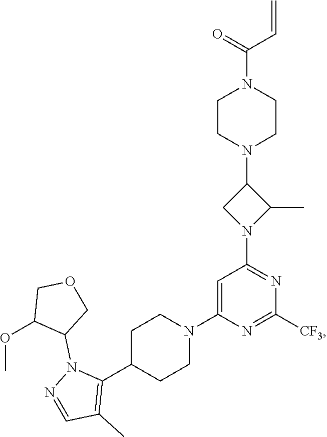 [SMILES: C=CC(=O)N1CCN(C2CN(c3cc(N4CCC(c5c(C)cnn5C5COCC5OC)CC4)nc(C(F)(F)F)n3)C2C)CC1]

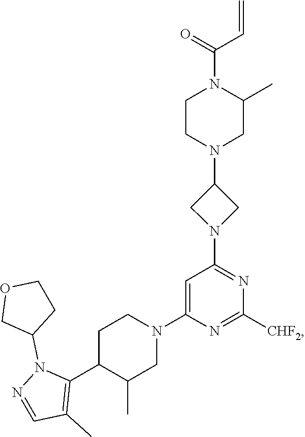 [SMILES: C=CC(=O)N1CCN(C2CN(c3cc(N4CCC(c5c(C)cnn5C5CCOC5)C(C)C4)nc(C(F)F)n3)C2)CC1C]

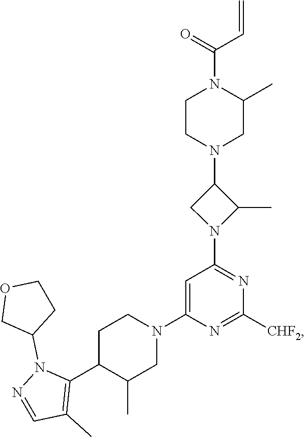 [SMILES: C=CC(=O)N1CCN(C2CN(c3cc(N4CCC(c5c(C)cnn5C5CCOC5)C(C)C4)nc(C(F)F)n3)C2C)CC1C]

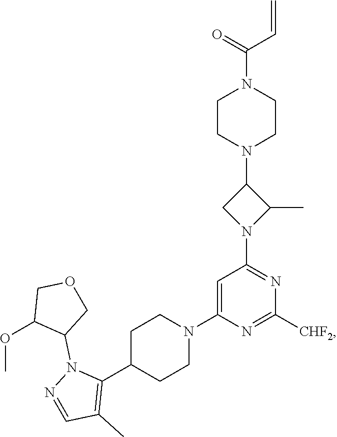 [SMILES: C=CC(=O)N1CCN(C2CN(c3cc(N4CCC(c5c(C)cnn5C5COCC5OC)CC4)nc(C(F)F)n3)C2C)CC1]

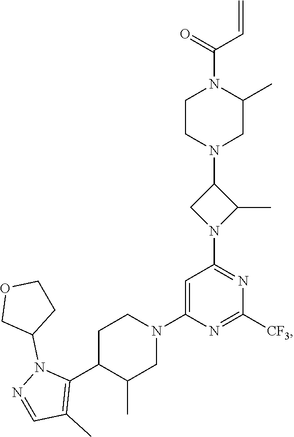 [SMILES: C=CC(=O)N1CCN(C2CN(c3cc(N4CCC(c5c(C)cnn5C5CCOC5)C(C)C4)nc(C(F)(F)F)n3)C2C)CC1C]

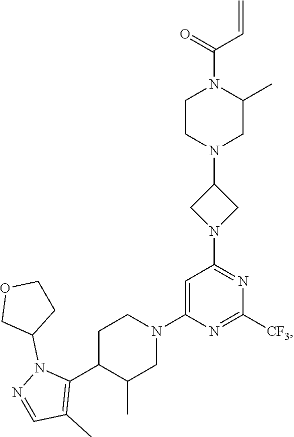 [SMILES: C=CC(=O)N1CCN(C2CN(c3cc(N4CCC(c5c(C)cnn5C5CCOC5)C(C)C4)nc(C(F)(F)F)n3)C2)CC1C]

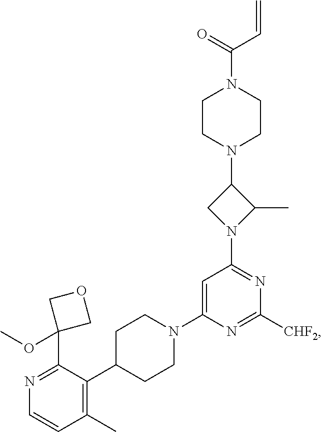 [SMILES: C=CC(=O)N1CCN(C2CN(c3cc(N4CCC(c5c(C)ccnc5C5(OC)COC5)CC4)nc(C(F)F)n3)C2C)CC1]

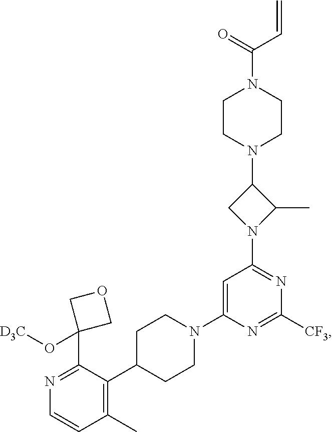 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2)COC1]

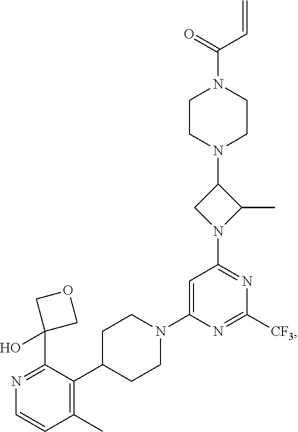 [SMILES: C=CC(=O)N1CCN(C2CN(c3cc(N4CCC(c5c(C)ccnc5C5(O)COC5)CC4)nc(C(F)(F)F)n3)C2C)CC1]

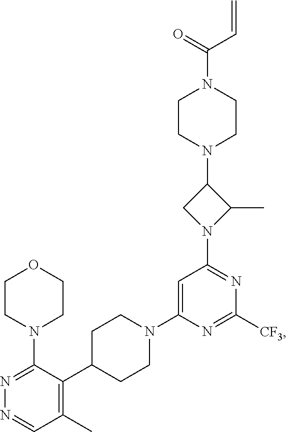 [SMILES: C=CC(=O)N1CCN(C2CN(c3cc(N4CCC(c5c(C)cnnc5N5CCOCC5)CC4)nc(C(F)(F)F)n3)C2C)CC1]

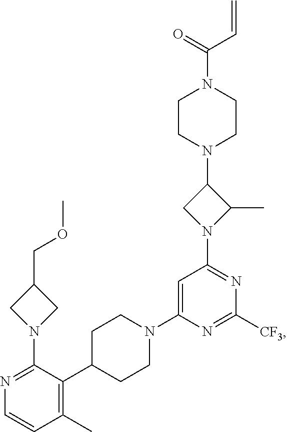 [SMILES: C=CC(=O)N1CCN(C2CN(c3cc(N4CCC(c5c(C)ccnc5N5CC(COC)C5)CC4)nc(C(F)(F)F)n3)C2C)CC1]

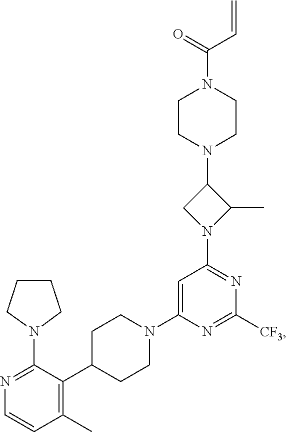 [SMILES: C=CC(=O)N1CCN(C2CN(c3cc(N4CCC(c5c(C)ccnc5N5CCCC5)CC4)nc(C(F)(F)F)n3)C2C)CC1]

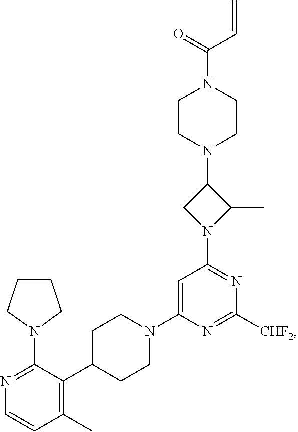 [SMILES: C=CC(=O)N1CCN(C2CN(c3cc(N4CCC(c5c(C)ccnc5N5CCCC5)CC4)nc(C(F)F)n3)C2C)CC1]

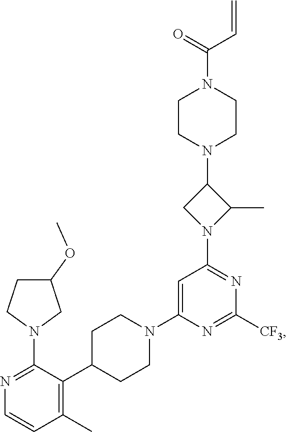 [SMILES: C=CC(=O)N1CCN(C2CN(c3cc(N4CCC(c5c(C)ccnc5N5CCC(OC)C5)CC4)nc(C(F)(F)F)n3)C2C)CC1]

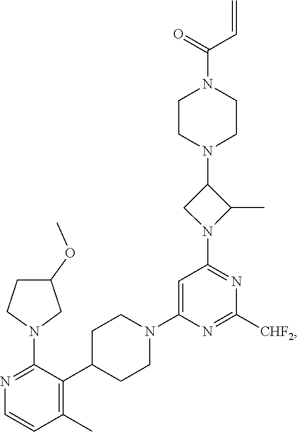 [SMILES: C=CC(=O)N1CCN(C2CN(c3cc(N4CCC(c5c(C)ccnc5N5CCC(OC)C5)CC4)nc(C(F)F)n3)C2C)CC1]

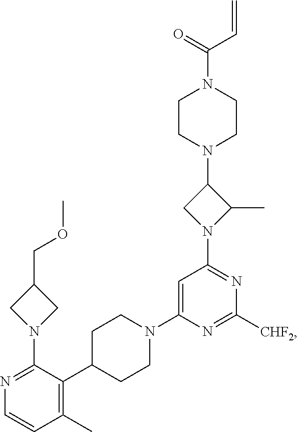 [SMILES: C=CC(=O)N1CCN(C2CN(c3cc(N4CCC(c5c(C)ccnc5N5CC(COC)C5)CC4)nc(C(F)F)n3)C2C)CC1]

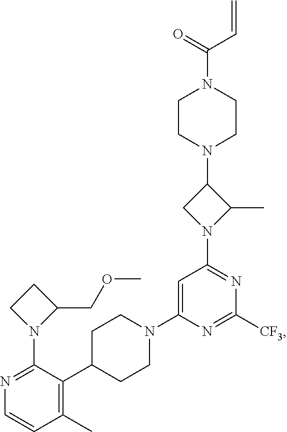 [SMILES: C=CC(=O)N1CCN(C2CN(c3cc(N4CCC(c5c(C)ccnc5N5CCC5COC)CC4)nc(C(F)(F)F)n3)C2C)CC1]

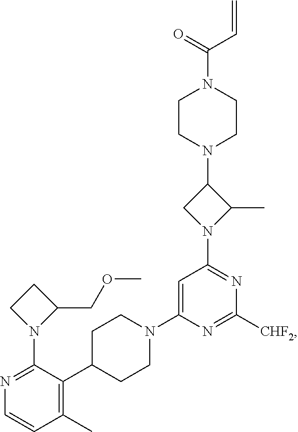 [SMILES: C=CC(=O)N1CCN(C2CN(c3cc(N4CCC(c5c(C)ccnc5N5CCC5COC)CC4)nc(C(F)F)n3)C2C)CC1]

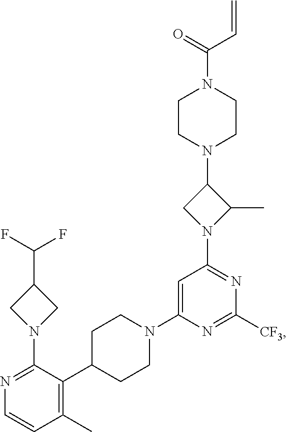 [SMILES: C=CC(=O)N1CCN(C2CN(c3cc(N4CCC(c5c(C)ccnc5N5CC(C(F)F)C5)CC4)nc(C(F)(F)F)n3)C2C)CC1]

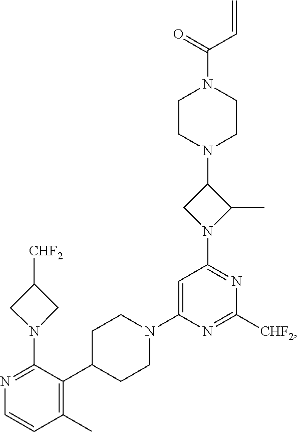 [SMILES: C=CC(=O)N1CCN(C2CN(c3cc(N4CCC(c5c(C)ccnc5N5CC(C(F)F)C5)CC4)nc(C(F)F)n3)C2C)CC1]

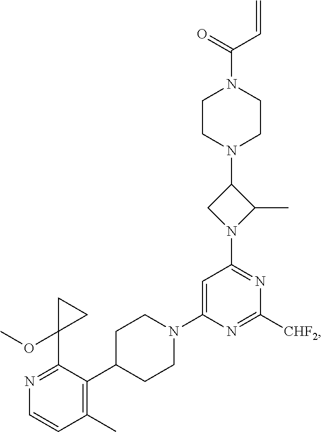 [SMILES: C=CC(=O)N1CCN(C2CN(c3cc(N4CCC(c5c(C)ccnc5C5(OC)CC5)CC4)nc(C(F)F)n3)C2C)CC1]

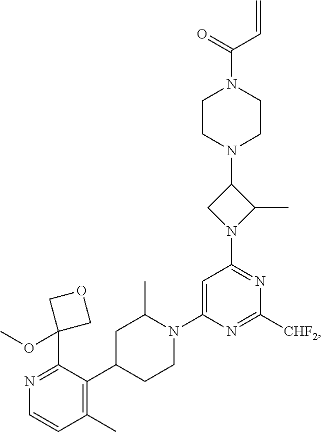 [SMILES: C=CC(=O)N1CCN(C2CN(c3cc(N4CCC(c5c(C)ccnc5C5(OC)COC5)CC4C)nc(C(F)F)n3)C2C)CC1]

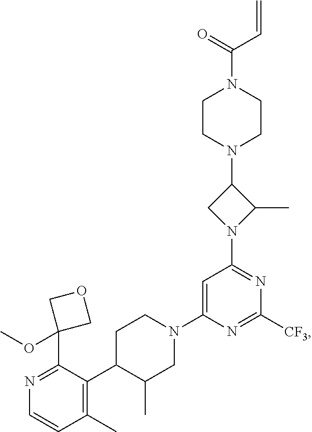 [SMILES: C=CC(=O)N1CCN(C2CN(c3cc(N4CCC(c5c(C)ccnc5C5(OC)COC5)C(C)C4)nc(C(F)(F)F)n3)C2C)CC1]

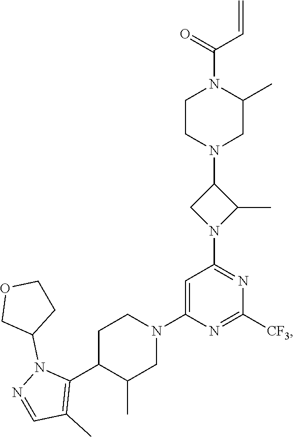 [SMILES: C=CC(=O)N1CCN(C2CN(c3cc(N4CCC(c5c(C)cnn5C5CCOC5)C(C)C4)nc(C(F)(F)F)n3)C2C)CC1C]

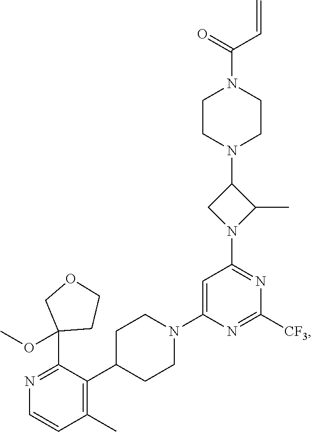 [SMILES: C=CC(=O)N1CCN(C2CN(c3cc(N4CCC(c5c(C)ccnc5C5(OC)CCOC5)CC4)nc(C(F)(F)F)n3)C2C)CC1]

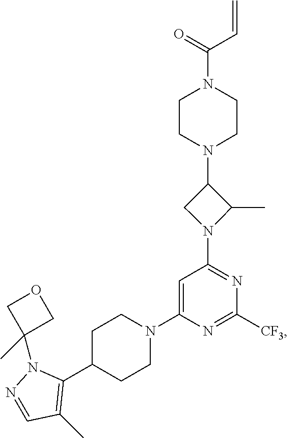 [SMILES: C=CC(=O)N1CCN(C2CN(c3cc(N4CCC(c5c(C)cnn5C5(C)COC5)CC4)nc(C(F)(F)F)n3)C2C)CC1]

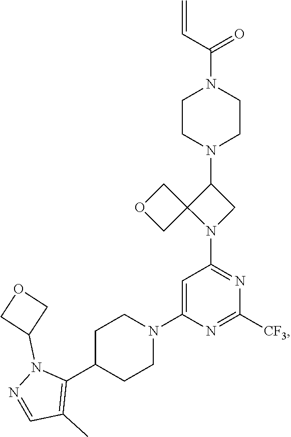 [SMILES: C=CC(=O)N1CCN(C2CN(c3cc(N4CCC(c5c(C)cnn5C5COC5)CC4)nc(C(F)(F)F)n3)C23COC3)CC1]

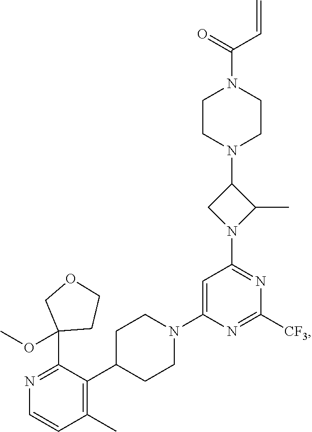 [SMILES: C=CC(=O)N1CCN(C2CN(c3cc(N4CCC(c5c(C)ccnc5C5(OC)CCOC5)CC4)nc(C(F)(F)F)n3)C2C)CC1]

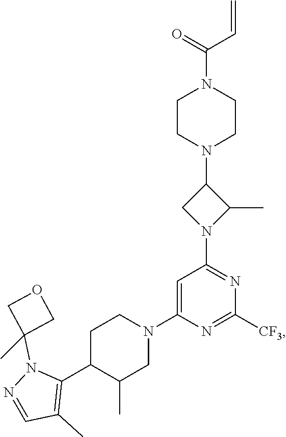 [SMILES: C=CC(=O)N1CCN(C2CN(c3cc(N4CCC(c5c(C)cnn5C5(C)COC5)C(C)C4)nc(C(F)(F)F)n3)C2C)CC1]

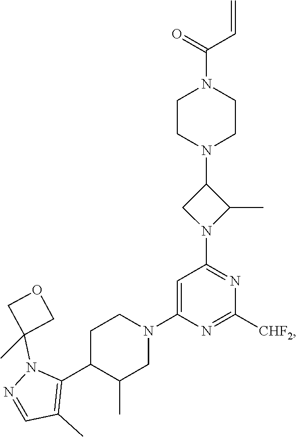 [SMILES: C=CC(=O)N1CCN(C2CN(c3cc(N4CCC(c5c(C)cnn5C5(C)COC5)C(C)C4)nc(C(F)F)n3)C2C)CC1]

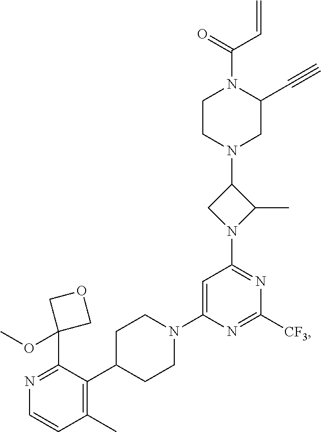 [SMILES: C#CC1CN(C2CN(c3cc(N4CCC(c5c(C)ccnc5C5(OC)COC5)CC4)nc(C(F)(F)F)n3)C2C)CCN1C(=O)C=C]

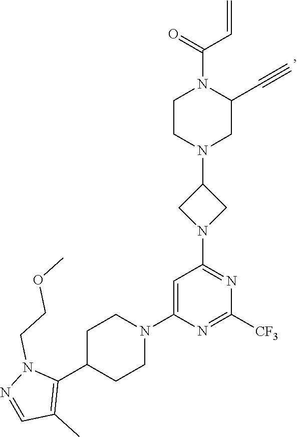 [SMILES: C#CC1CN(C2CN(c3cc(N4CCC(c5c(C)cnn5CCOC)CC4)nc(C(F)(F)F)n3)C2)CCN1C(=O)C=C]

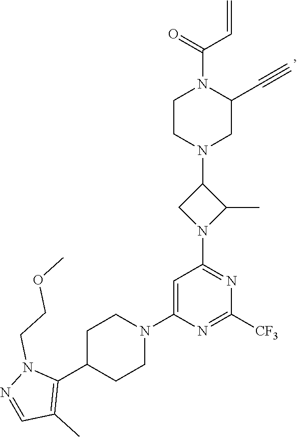 [SMILES: C#CC1CN(C2CN(c3cc(N4CCC(c5c(C)cnn5CCOC)CC4)nc(C(F)(F)F)n3)C2C)CCN1C(=O)C=C]

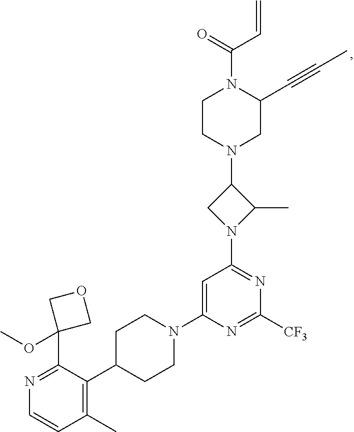 [SMILES: C=CC(=O)N1CCN(C2CN(c3cc(N4CCC(c5c(C)ccnc5C5(OC)COC5)CC4)nc(C(F)(F)F)n3)C2C)CC1C#CC]

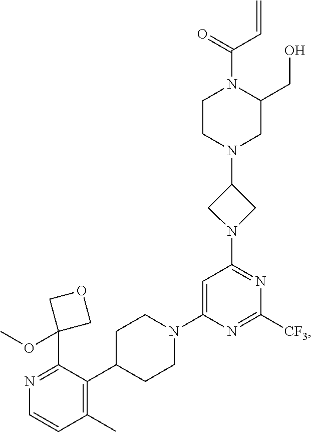 [SMILES: C=CC(=O)N1CCN(C2CN(c3cc(N4CCC(c5c(C)ccnc5C5(OC)COC5)CC4)nc(C(F)(F)F)n3)C2)CC1CO]

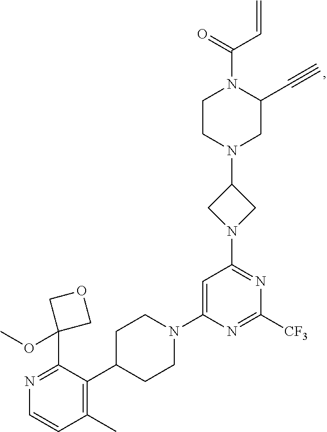 [SMILES: C#CC1CN(C2CN(c3cc(N4CCC(c5c(C)ccnc5C5(OC)COC5)CC4)nc(C(F)(F)F)n3)C2)CCN1C(=O)C=C]

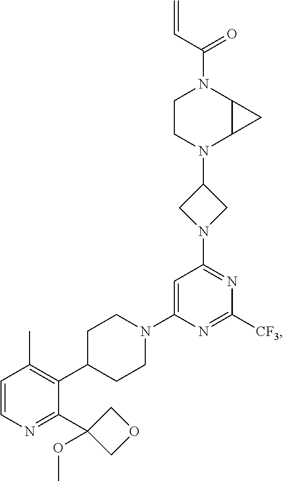 [SMILES: C=CC(=O)N1CCN(C2CN(c3cc(N4CCC(c5c(C)ccnc5C5(OC)COC5)CC4)nc(C(F)(F)F)n3)C2)C2CC21]

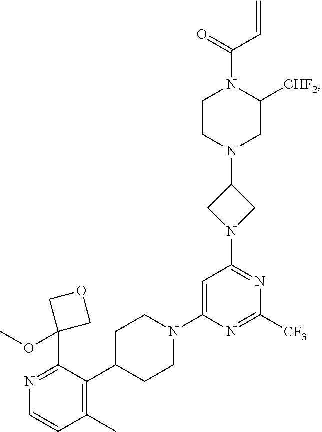 [SMILES: C=CC(=O)N1CCN(C2CN(c3cc(N4CCC(c5c(C)ccnc5C5(OC)COC5)CC4)nc(C(F)(F)F)n3)C2)CC1C(F)F]

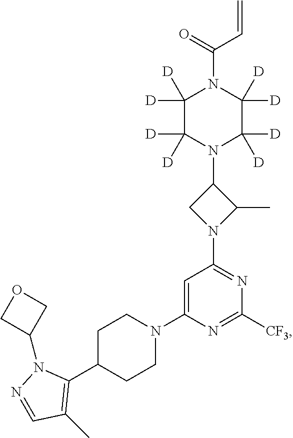 [SMILES: [2H]C1([2H])N(C(=O)C=C)C([2H])([2H])C([2H])([2H])N(C2CN(c3cc(N4CCC(c5c(C)cnn5C5COC5)CC4)nc(C(F)(F)F)n3)C2C)C1([2H])[2H]]

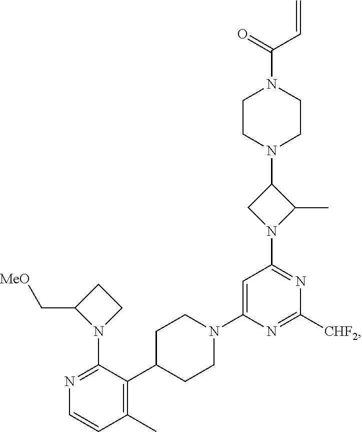 [SMILES: C=CC(=O)N1CCN(C2CN(c3cc(N4CCC(c5c(C)ccnc5N5CCC5COC)CC4)nc(C(F)F)n3)C2C)CC1]

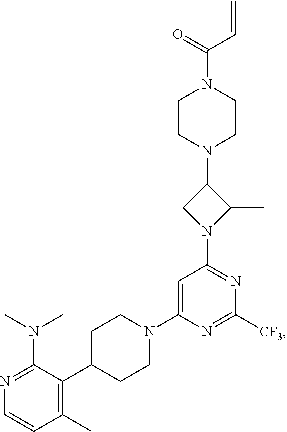 [SMILES: C=CC(=O)N1CCN(C2CN(c3cc(N4CCC(c5c(C)ccnc5N(C)C)CC4)nc(C(F)(F)F)n3)C2C)CC1]

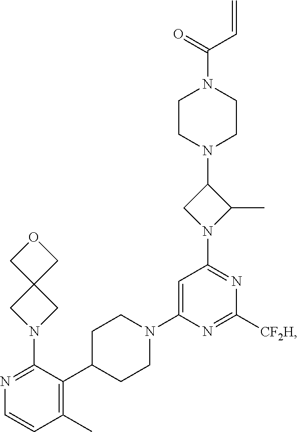 [SMILES: C=CC(=O)N1CCN(C2CN(c3cc(N4CCC(c5c(C)ccnc5N5CC6(COC6)C5)CC4)nc(C(F)F)n3)C2C)CC1]

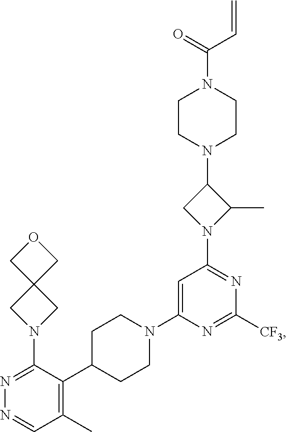 [SMILES: C=CC(=O)N1CCN(C2CN(c3cc(N4CCC(c5c(C)cnnc5N5CC6(COC6)C5)CC4)nc(C(F)(F)F)n3)C2C)CC1]

In some cases, the compound of Formula (I) is selected from the list of compounds in Table B, or a pharmaceutically acceptable salt thereof.

In some cases, A is N; X is

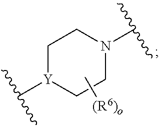

and Y is C—H, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy, and the disclosure provides compounds of Formula (IC):

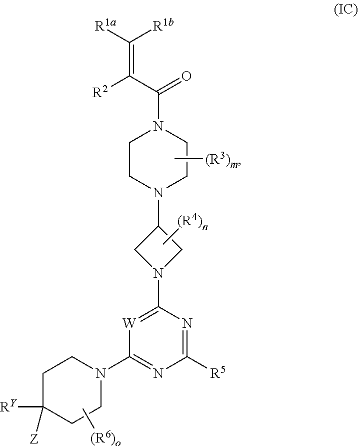

and pharmaceutically acceptable salts thereof, wherein R^{Y} is H, halo, CN, C₁₋₃ alkyl, C₁₋₃ haloalkyl, C₀₋₃ alkyleneOH, or C₀₋₃ alkylene-C₁₋₄ alkoxy and the remaining substituents are as previously defined herein, Contemplated compounds of Formula (IC) include, but are not limited to, those listed in Table C, below, and pharmaceutically acceptable salts thereof.

TABLE C

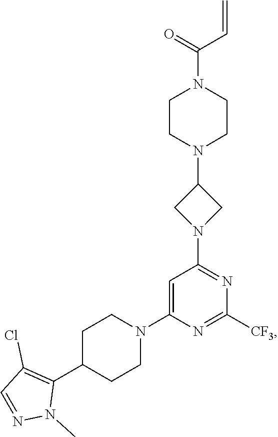 [SMILES: C=CC(=O)N1CCN(C2CN(c3cc(N4CCC(c5c(Cl)cnn5C)CC4)nc(C(F)(F)F)n3)C2)CC1]

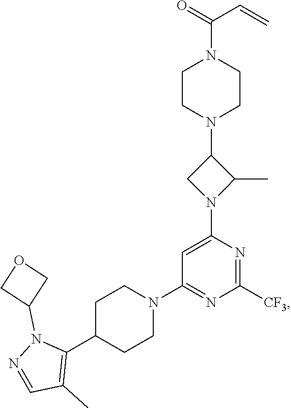 [SMILES: C=CC(=O)N1CCN(C2CN(c3cc(N4CCC(c5c(C)cnn5C5COC5)CC4)nc(C(F)(F)F)n3)C2C)CC1]

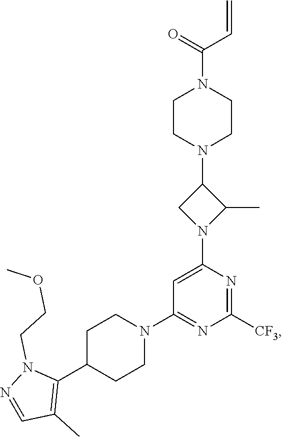 [SMILES: C=CC(=O)N1CCN(C2CN(c3cc(N4CCC(c5c(C)cnn5CCOC)CC4)nc(C(F)(F)F)n3)C2C)CC1]

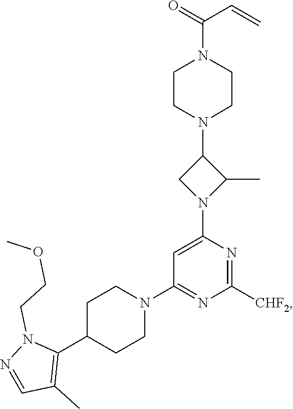 [SMILES: C=CC(=O)N1CCN(C2CN(c3cc(N4CCC(c5c(C)cnn5CCOC)CC4)nc(C(F)F)n3)C2C)CC1]

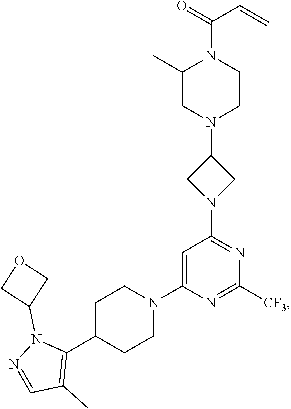 [SMILES: C=CC(=O)N1CCN(C2CN(c3cc(N4CCC(c5c(C)cnn5C5COC5)CC4)nc(C(F)(F)F)n3)C2)CC1C]

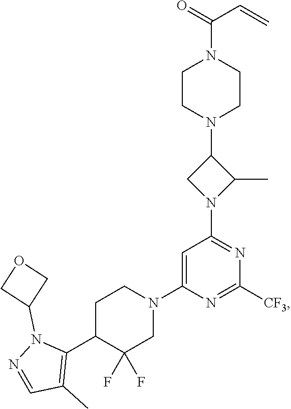 [SMILES: C=CC(=O)N1CCN(C2CN(c3cc(N4CCC(c5c(C)cnn5C5COC5)C(F)(F)C4)nc(C(F)(F)F)n3)C2C)CC1]

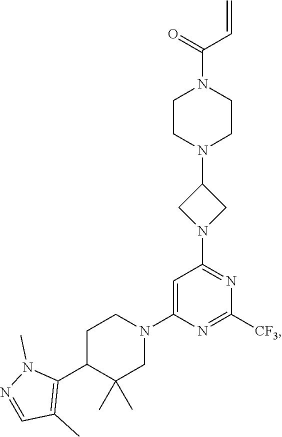 [SMILES: C=CC(=O)N1CCN(C2CN(c3cc(N4CCC(c5c(C)cnn5C)C(C)(C)C4)nc(C(F)(F)F)n3)C2)CC1]

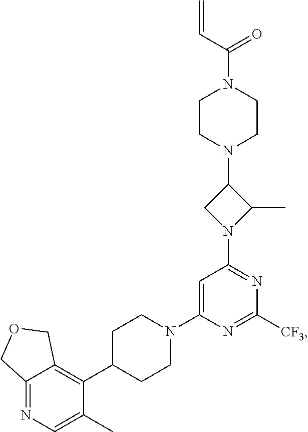 [SMILES: C=CC(=O)N1CCN(C2CN(c3cc(N4CCC(c5c(C)cnc6c5COC6)CC4)nc(C(F)(F)F)n3)C2C)CC1]

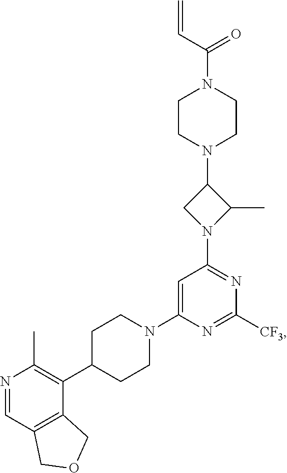 [SMILES: C=CC(=O)N1CCN(C2CN(c3cc(N4CCC(c5c(C)ncc6c5COC6)CC4)nc(C(F)(F)F)n3)C2C)CC1]

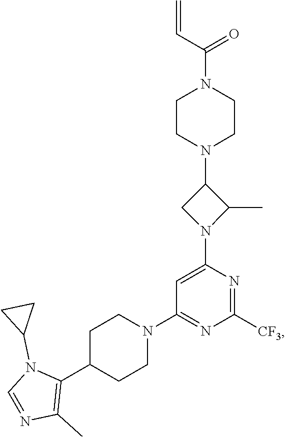 [SMILES: C=CC(=O)N1CCN(C2CN(c3cc(N4CCC(c5c(C)ncn5C5CC5)CC4)nc(C(F)(F)F)n3)C2C)CC1]

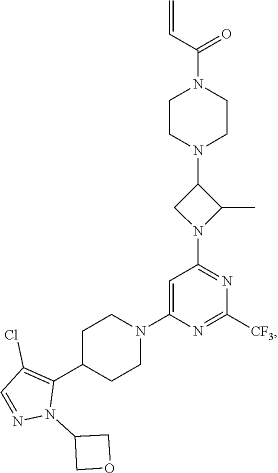 [SMILES: C=CC(=O)N1CCN(C2CN(c3cc(N4CCC(c5c(Cl)cnn5C5COC5)CC4)nc(C(F)(F)F)n3)C2C)CC1]

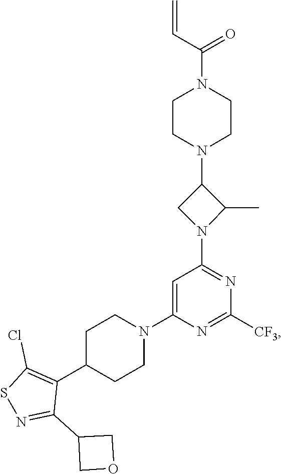 [SMILES: C=CC(=O)N1CCN(C2CN(c3cc(N4CCC(c5c(C6COC6)nsc5Cl)CC4)nc(C(F)(F)F)n3)C2C)CC1]

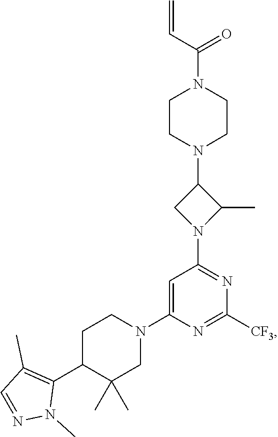 [SMILES: C=CC(=O)N1CCN(C2CN(c3cc(N4CCC(c5c(C)cnn5C)C(C)(C)C4)nc(C(F)(F)F)n3)C2C)CC1]

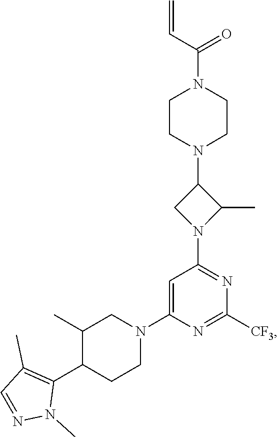 [SMILES: C=CC(=O)N1CCN(C2CN(c3cc(N4CCC(c5c(C)cnn5C)C(C)C4)nc(C(F)(F)F)n3)C2C)CC1]

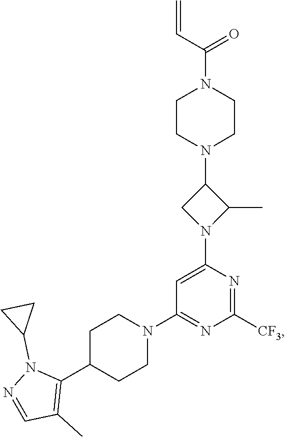 [SMILES: C=CC(=O)N1CCN(C2CN(c3cc(N4CCC(c5c(C)cnn5C5CC5)CC4)nc(C(F)(F)F)n3)C2C)CC1]

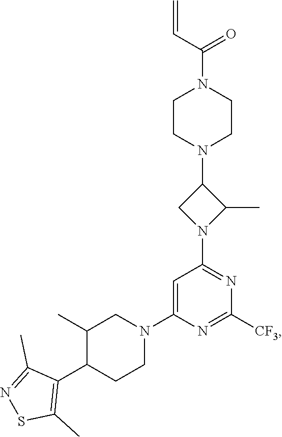 [SMILES: C=CC(=O)N1CCN(C2CN(c3cc(N4CCC(c5c(C)nsc5C)C(C)C4)nc(C(F)(F)F)n3)C2C)CC1]

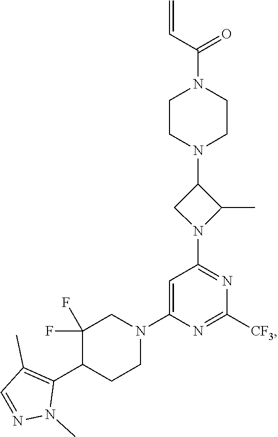 [SMILES: C=CC(=O)N1CCN(C2CN(c3cc(N4CCC(c5c(C)cnn5C)C(F)(F)C4)nc(C(F)(F)F)n3)C2C)CC1]

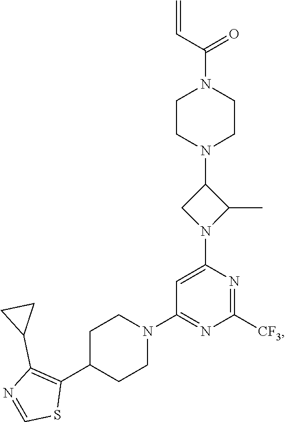 [SMILES: C=CC(=O)N1CCN(C2CN(c3cc(N4CCC(c5scnc5C5CC5)CC4)nc(C(F)(F)F)n3)C2C)CC1]

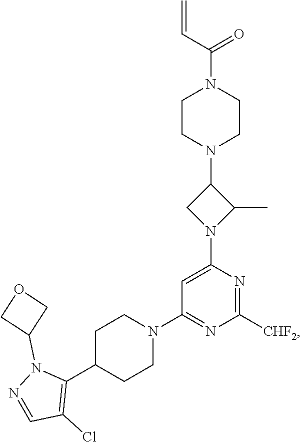 [SMILES: C=CC(=O)N1CCN(C2CN(c3cc(N4CCC(c5c(Cl)cnn5C5COC5)CC4)nc(C(F)F)n3)C2C)CC1]

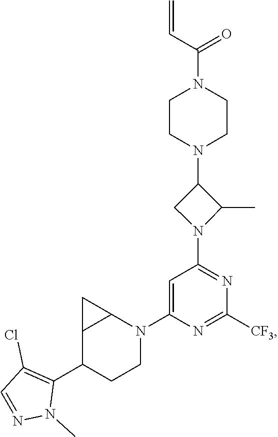 [SMILES: C=CC(=O)N1CCN(C2CN(c3cc(N4CCC(c5c(Cl)cnn5C)C5CC54)nc(C(F)(F)F)n3)C2C)CC1]

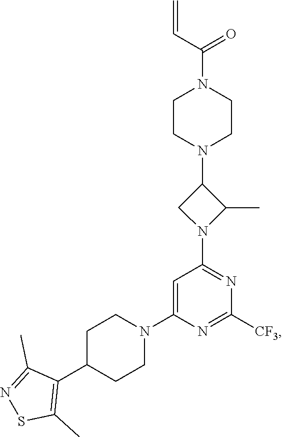 [SMILES: C=CC(=O)N1CCN(C2CN(c3cc(N4CCC(c5c(C)nsc5C)CC4)nc(C(F)(F)F)n3)C2C)CC1]

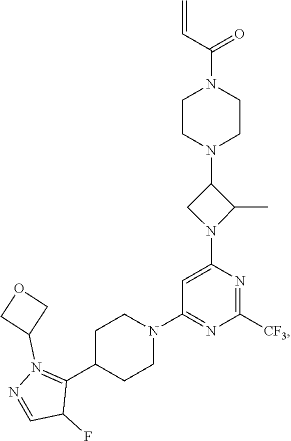

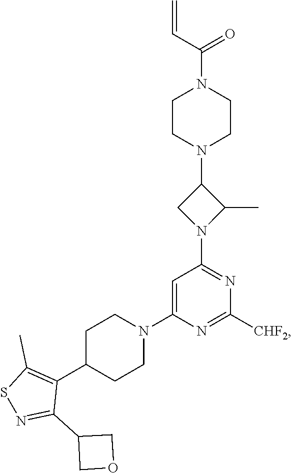 [SMILES: C=CC(=O)N1CCN(C2CN(c3cc(N4CCC(c5c(C6COC6)nsc5C)CC4)nc(C(F)F)n3)C2C)CC1]

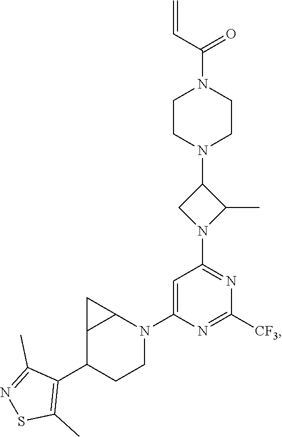 [SMILES: C=CC(=O)N1CCN(C2CN(c3cc(N4CCC(c5c(C)nsc5C)C5CC54)nc(C(F)(F)F)n3)C2C)CC1]

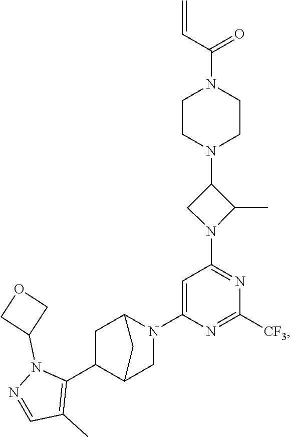 [SMILES: C=CC(=O)N1CCN(C2CN(c3cc(N4CC5CC4CC5c4c(C)cnn4C4COC4)nc(C(F)(F)F)n3)C2C)CC1]

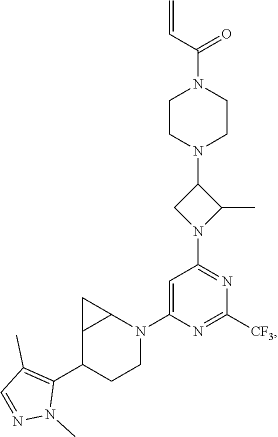 [SMILES: C=CC(=O)N1CCN(C2CN(c3cc(N4CCC(c5c(C)cnn5C)C5CC54)nc(C(F)(F)F)n3)C2C)CC1]

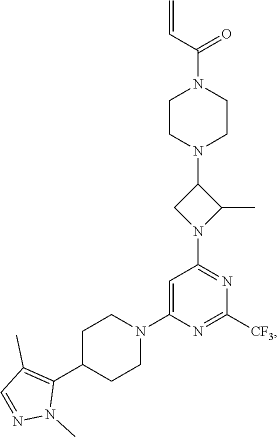 [SMILES: C=CC(=O)N1CCN(C2CN(c3cc(N4CCC(c5c(C)cnn5C)CC4)nc(C(F)(F)F)n3)C2C)CC1]

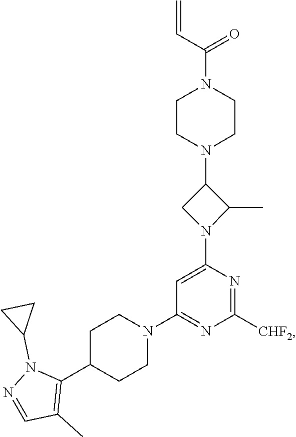 [SMILES: C=CC(=O)N1CCN(C2CN(c3cc(N4CCC(c5c(C)cnn5C5CC5)CC4)nc(C(F)F)n3)C2C)CC1]

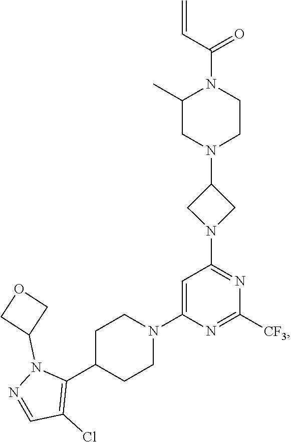 [SMILES: C=CC(=O)N1CCN(C2CN(c3cc(N4CCC(c5c(Cl)cnn5C5COC5)CC4)nc(C(F)(F)F)n3)C2)CC1C]

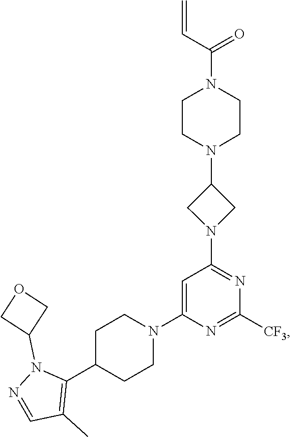 [SMILES: C=CC(=O)N1CCN(C2CN(c3cc(N4CCC(c5c(C)cnn5C5COC5)CC4)nc(C(F)(F)F)n3)C2)CC1]

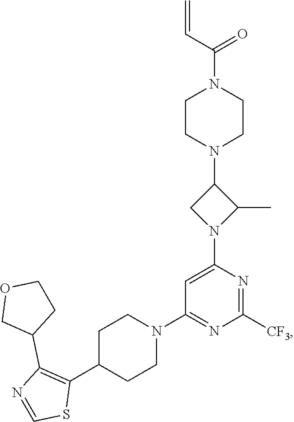 [SMILES: C=CC(=O)N1CCN(C2CN(c3cc(N4CCC(c5scnc5C5CCOC5)CC4)nc(C(F)(F)F)n3)C2C)CC1]

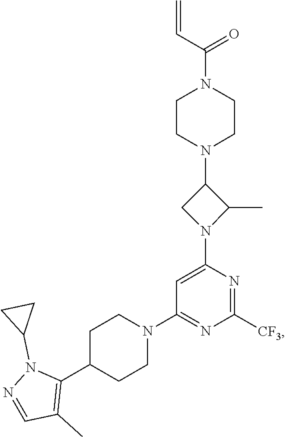 [SMILES: C=CC(=O)N1CCN(C2CN(c3cc(N4CCC(c5c(C)cnn5C5CC5)CC4)nc(C(F)(F)F)n3)C2C)CC1]

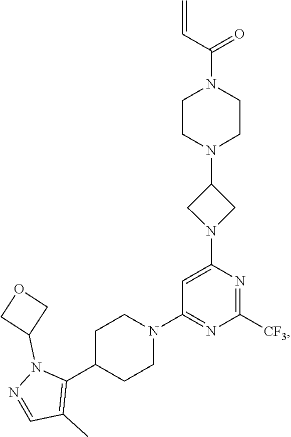 [SMILES: C=CC(=O)N1CCN(C2CN(c3cc(N4CCC(c5c(C)cnn5C5COC5)CC4)nc(C(F)(F)F)n3)C2)CC1]

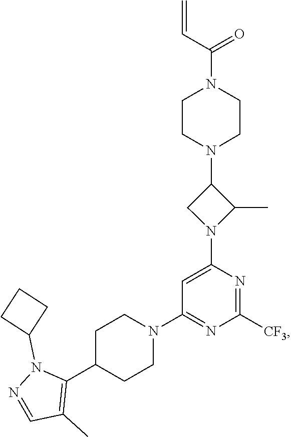 [SMILES: C=CC(=O)N1CCN(C2CN(c3cc(N4CCC(c5c(C)cnn5C5CCC5)CC4)nc(C(F)(F)F)n3)C2C)CC1]

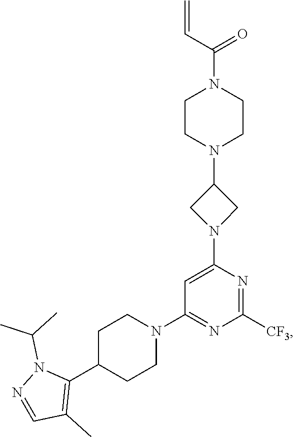 [SMILES: C=CC(=O)N1CCN(C2CN(c3cc(N4CCC(c5c(C)cnn5C(C)C)CC4)nc(C(F)(F)F)n3)C2)CC1]

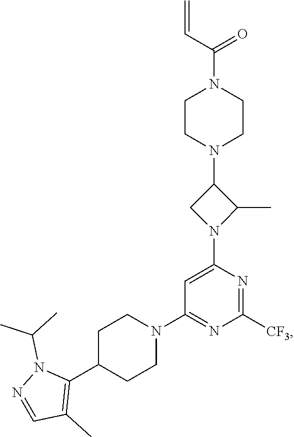 [SMILES: C=CC(=O)N1CCN(C2CN(c3cc(N4CCC(c5c(C)cnn5C(C)C)CC4)nc(C(F)(F)F)n3)C2C)CC1]

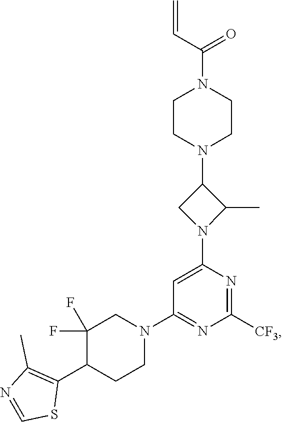 [SMILES: C=CC(=O)N1CCN(C2CN(c3cc(N4CCC(c5scnc5C)C(F)(F)C4)nc(C(F)(F)F)n3)C2C)CC1]

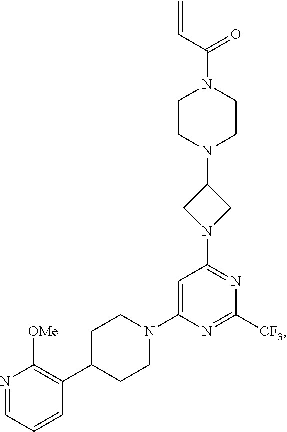 [SMILES: C=CC(=O)N1CCN(C2CN(c3cc(N4CCC(c5cccnc5OC)CC4)nc(C(F)(F)F)n3)C2)CC1]

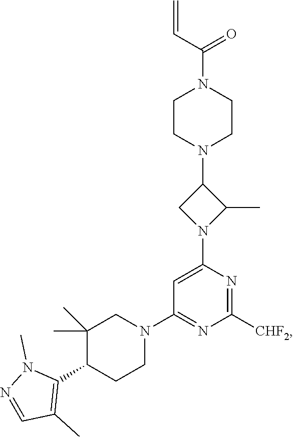 [SMILES: C=CC(=O)N1CCN(C2CN(c3cc(N4CC[C@H](c5c(C)cnn5C)C(C)(C)C4)nc(C(F)F)n3)C2C)CC1]

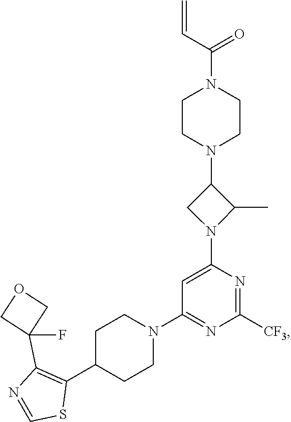 [SMILES: C=CC(=O)N1CCN(C2CN(c3cc(N4CCC(c5scnc5C5(F)COC5)CC4)nc(C(F)(F)F)n3)C2C)CC1]

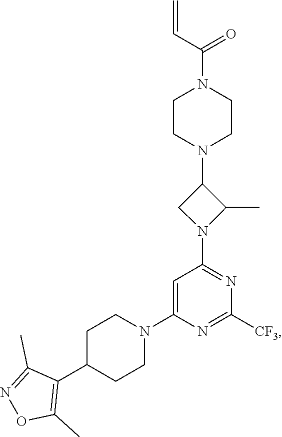 [SMILES: C=CC(=O)N1CCN(C2CN(c3cc(N4CCC(c5c(C)noc5C)CC4)nc(C(F)(F)F)n3)C2C)CC1]

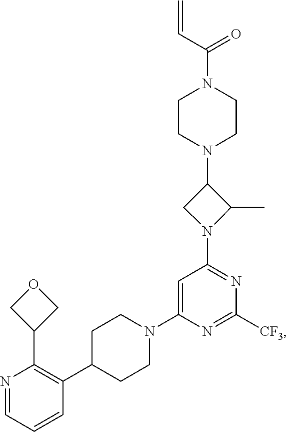 [SMILES: C=CC(=O)N1CCN(C2CN(c3cc(N4CCC(c5cccnc5C5COC5)CC4)nc(C(F)(F)F)n3)C2C)CC1]

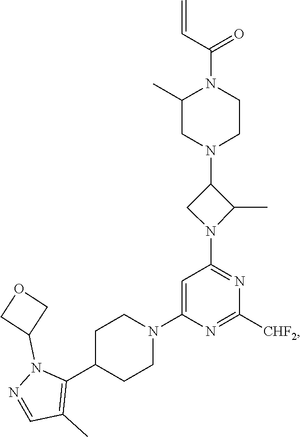 [SMILES: C=CC(=O)N1CCN(C2CN(c3cc(N4CCC(c5c(C)cnn5C5COC5)CC4)nc(C(F)F)n3)C2C)CC1C]

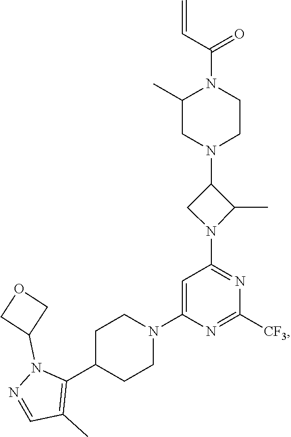 [SMILES: C=CC(=O)N1CCN(C2CN(c3cc(N4CCC(c5c(C)cnn5C5COC5)CC4)nc(C(F)(F)F)n3)C2C)CC1C]

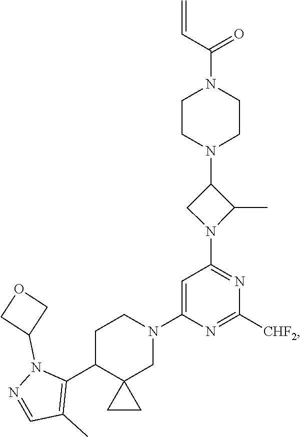 [SMILES: C=CC(=O)N1CCN(C2CN(c3cc(N4CCC(c5c(C)cnn5C5COC5)C5(CC5)C4)nc(C(F)F)n3)C2C)CC1]

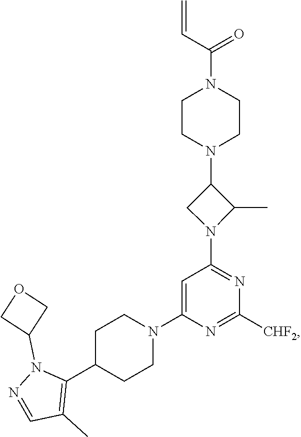 [SMILES: C=CC(=O)N1CCN(C2CN(c3cc(N4CCC(c5c(C)cnn5C5COC5)CC4)nc(C(F)F)n3)C2C)CC1]

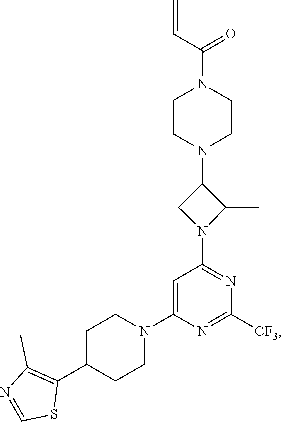 [SMILES: C=CC(=O)N1CCN(C2CN(c3cc(N4CCC(c5scnc5C)CC4)nc(C(F)(F)F)n3)C2C)CC1]

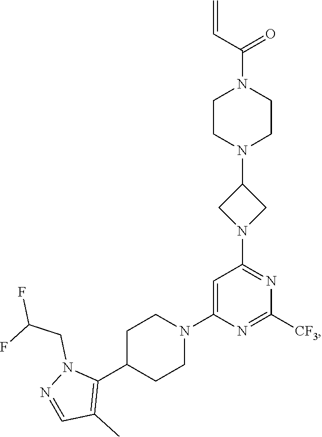 [SMILES: C=CC(=O)N1CCN(C2CN(c3cc(N4CCC(c5c(C)cnn5CC(F)F)CC4)nc(C(F)(F)F)n3)C2)CC1]

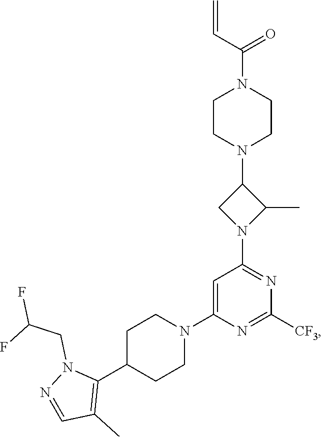 [SMILES: C=CC(=O)N1CCN(C2CN(c3cc(N4CCC(c5c(C)cnn5CC(F)F)CC4)nc(C(F)(F)F)n3)C2C)CC1]

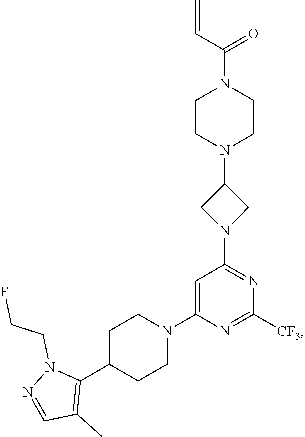 [SMILES: C=CC(=O)N1CCN(C2CN(c3cc(N4CCC(c5c(C)cnn5CCF)CC4)nc(C(F)(F)F)n3)C2)CC1]

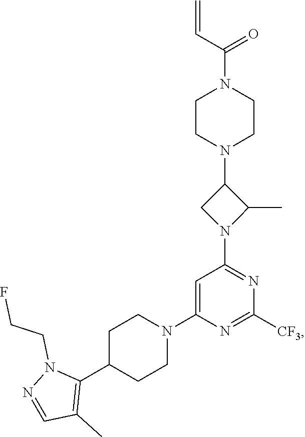 [SMILES: C=CC(=O)N1CCN(C2CN(c3cc(N4CCC(c5c(C)cnn5CCF)CC4)nc(C(F)(F)F)n3)C2C)CC1]

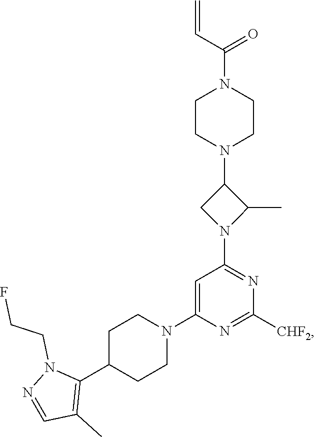 [SMILES: C=CC(=O)N1CCN(C2CN(c3cc(N4CCC(c5c(C)cnn5CCF)CC4)nc(C(F)F)n3)C2C)CC1]

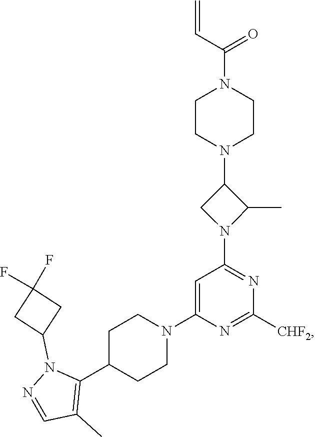 [SMILES: C=CC(=O)N1CCN(C2CN(c3cc(N4CCC(c5c(C)cnn5C5CC(F)(F)C5)CC4)nc(C(F)F)n3)C2C)CC1]

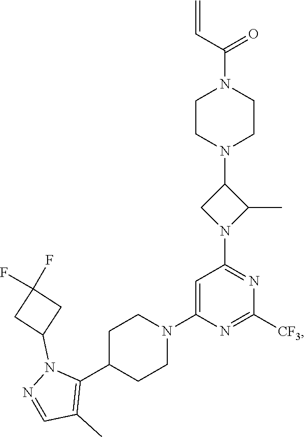 [SMILES: C=CC(=O)N1CCN(C2CN(c3cc(N4CCC(c5c(C)cnn5C5CC(F)(F)C5)CC4)nc(C(F)(F)F)n3)C2C)CC1]

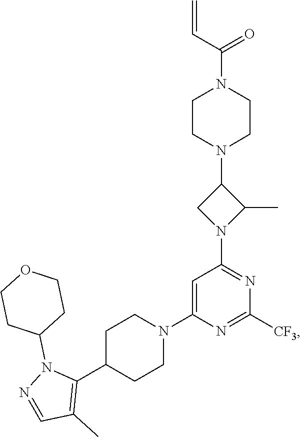 [SMILES: C=CC(=O)N1CCN(C2CN(c3cc(N4CCC(c5c(C)cnn5C5CCOCC5)CC4)nc(C(F)(F)F)n3)C2C)CC1]

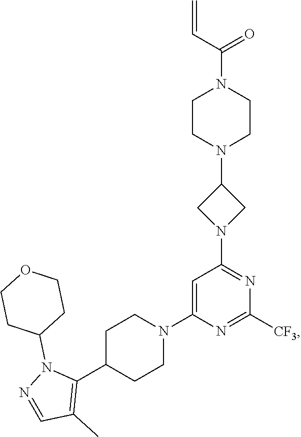 [SMILES: C=CC(=O)N1CCN(C2CN(c3cc(N4CCC(c5c(C)cnn5C5CCOCC5)CC4)nc(C(F)(F)F)n3)C2)CC1]

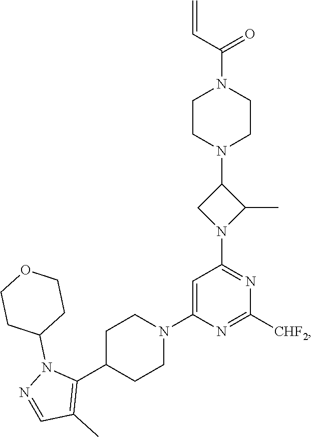 [SMILES: C=CC(=O)N1CCN(C2CN(c3cc(N4CCC(c5c(C)cnn5C5CCOCC5)CC4)nc(C(F)F)n3)C2C)CC1]

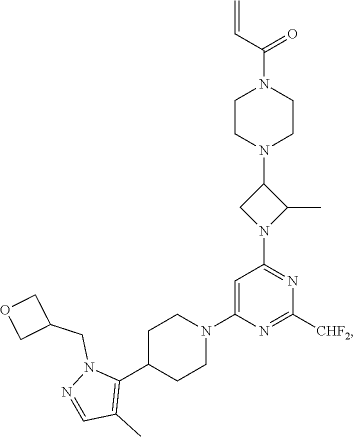 [SMILES: C=CC(=O)N1CCN(C2CN(c3cc(N4CCC(c5c(C)cnn5CC5COC5)CC4)nc(C(F)F)n3)C2C)CC1]

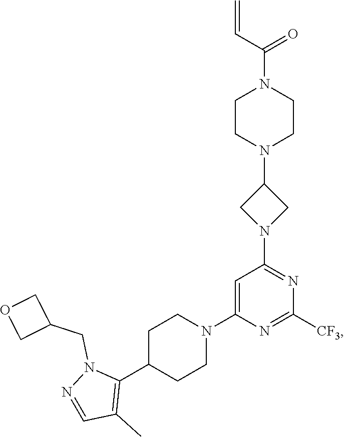 [SMILES: C=CC(=O)N1CCN(C2CN(c3cc(N4CCC(c5c(C)cnn5CC5COC5)CC4)nc(C(F)(F)F)n3)C2)CC1]

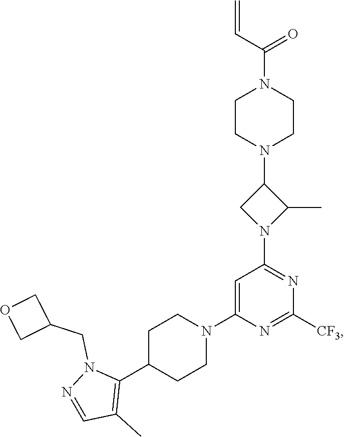 [SMILES: C=CC(=O)N1CCN(C2CN(c3cc(N4CCC(c5c(C)cnn5CC5COC5)CC4)nc(C(F)(F)F)n3)C2C)CC1]

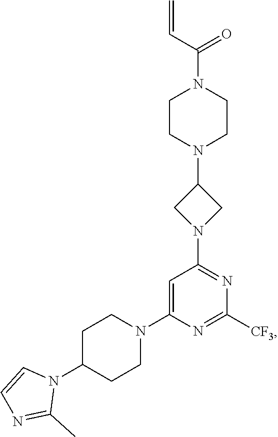 [SMILES: C=CC(=O)N1CCN(C2CN(c3cc(N4CCC(n5ccnc5C)CC4)nc(C(F)(F)F)n3)C2)CC1]

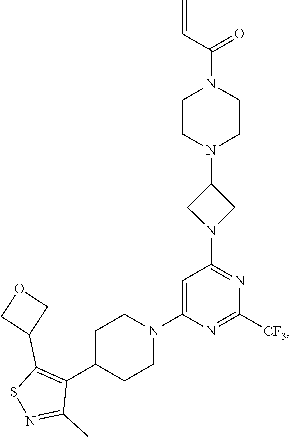 [SMILES: C=CC(=O)N1CCN(C2CN(c3cc(N4CCC(c5c(C)nsc5C5COC5)CC4)nc(C(F)(F)F)n3)C2)CC1]

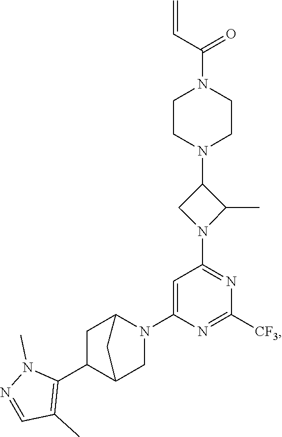 [SMILES: C=CC(=O)N1CCN(C2CN(c3cc(N4CC5CC4CC5c4c(C)cnn4C)nc(C(F)(F)F)n3)C2C)CC1]

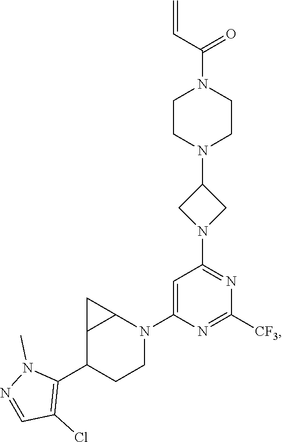 [SMILES: C=CC(=O)N1CCN(C2CN(c3cc(N4CCC(c5c(Cl)cnn5C)C5CC54)nc(C(F)(F)F)n3)C2)CC1]

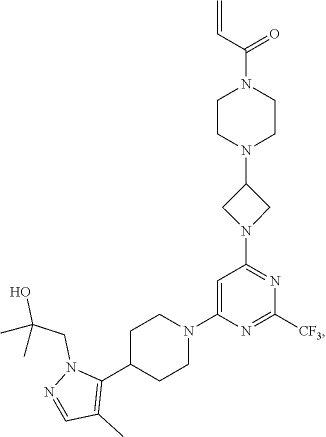 [SMILES: C=CC(=O)N1CCN(C2CN(c3cc(N4CCC(c5c(C)cnn5CC(C)(C)O)CC4)nc(C(F)(F)F)n3)C2)CC1]

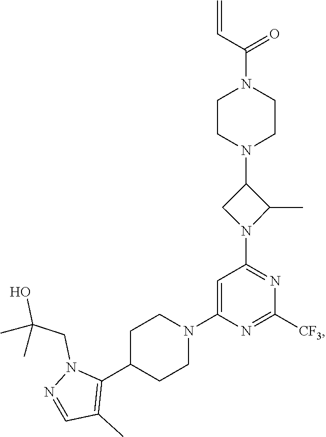 [SMILES: C=CC(=O)N1CCN(C2CN(c3cc(N4CCC(c5c(C)cnn5CC(C)(C)O)CC4)nc(C(F)(F)F)n3)C2C)CC1]

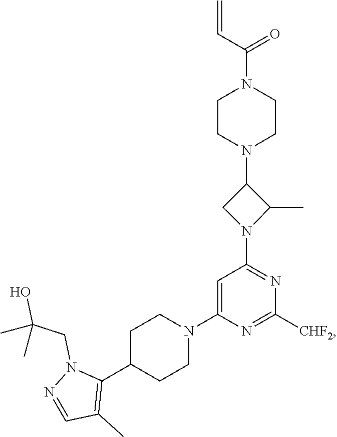 [SMILES: C=CC(=O)N1CCN(C2CN(c3cc(N4CCC(c5c(C)cnn5CC(C)(C)O)CC4)nc(C(F)F)n3)C2C)CC1]

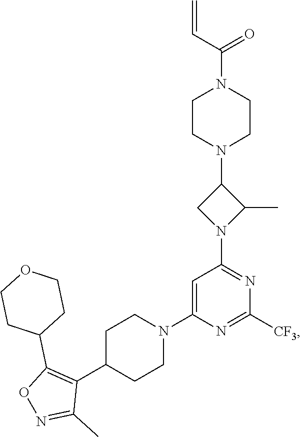 [SMILES: C=CC(=O)N1CCN(C2CN(c3cc(N4CCC(c5c(C)noc5C5CCOCC5)CC4)nc(C(F)(F)F)n3)C2C)CC1]

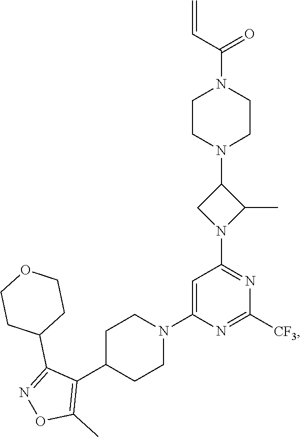 [SMILES: C=CC(=O)N1CCN(C2CN(c3cc(N4CCC(c5c(C6CCOCC6)noc5C)CC4)nc(C(F)(F)F)n3)C2C)CC1]

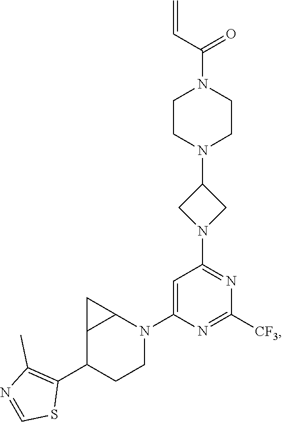 [SMILES: C=CC(=O)N1CCN(C2CN(c3cc(N4CCC(c5scnc5C)C5CC54)nc(C(F)(F)F)n3)C2)CC1]

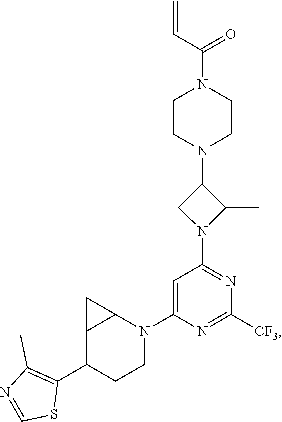 [SMILES: C=CC(=O)N1CCN(C2CN(c3cc(N4CCC(c5scnc5C)C5CC54)nc(C(F)(F)F)n3)C2C)CC1]

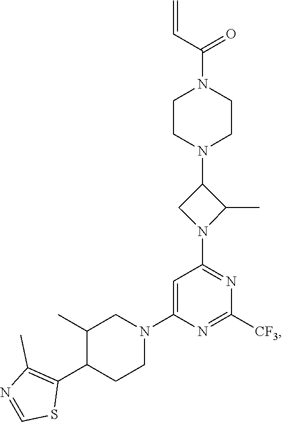 [SMILES: C=CC(=O)N1CCN(C2CN(c3cc(N4CCC(c5scnc5C)C(C)C4)nc(C(F)(F)F)n3)C2C)CC1]

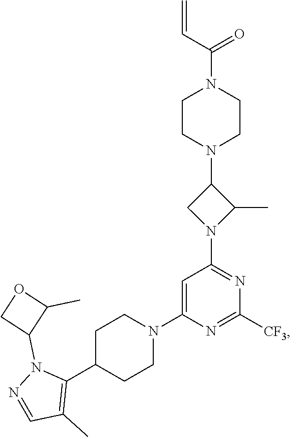 [SMILES: C=CC(=O)N1CCN(C2CN(c3cc(N4CCC(c5c(C)cnn5C5COC5C)CC4)nc(C(F)(F)F)n3)C2C)CC1]

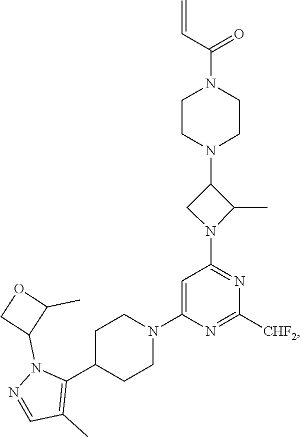 [SMILES: C=CC(=O)N1CCN(C2CN(c3cc(N4CCC(c5c(C)cnn5C5COC5C)CC4)nc(C(F)F)n3)C2C)CC1]

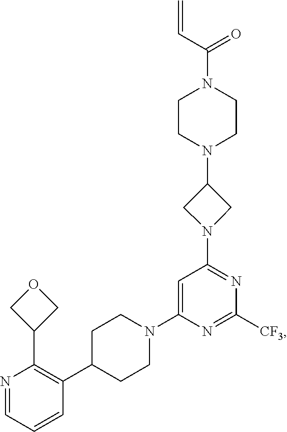 [SMILES: C=CC(=O)N1CCN(C2CN(c3cc(N4CCC(c5cccnc5C5COC5)CC4)nc(C(F)(F)F)n3)C2)CC1]

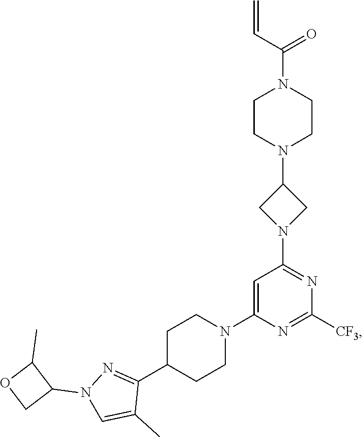 [SMILES: C=CC(=O)N1CCN(C2CN(c3cc(N4CCC(c5nn(C6COC6C)cc5C)CC4)nc(C(F)(F)F)n3)C2)CC1]

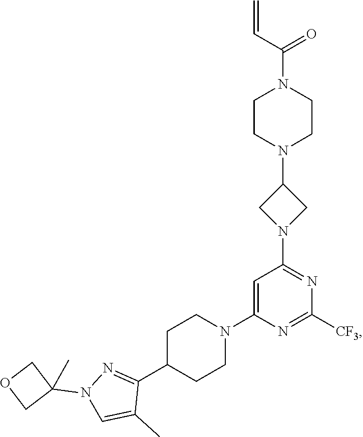 [SMILES: C=CC(=O)N1CCN(C2CN(c3cc(N4CCC(c5nn(C6(C)COC6)cc5C)CC4)nc(C(F)(F)F)n3)C2)CC1]

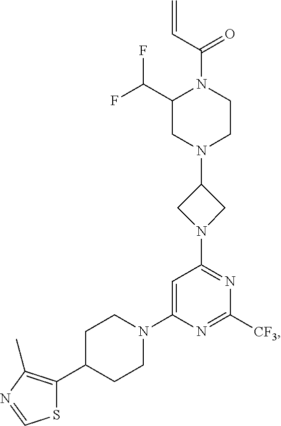 [SMILES: C=CC(=O)N1CCN(C2CN(c3cc(N4CCC(c5scnc5C)CC4)nc(C(F)(F)F)n3)C2)CC1C(F)F]

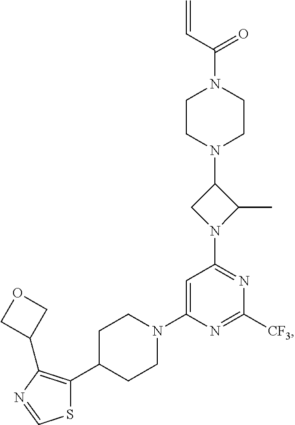 [SMILES: C=CC(=O)N1CCN(C2CN(c3cc(N4CCC(c5scnc5C5COC5)CC4)nc(C(F)(F)F)n3)C2C)CC1]

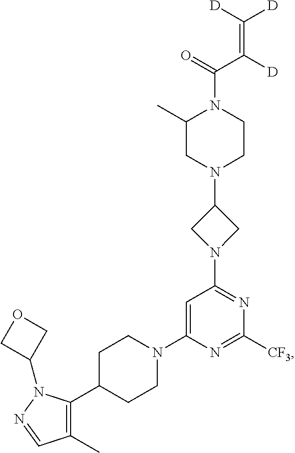 [SMILES: [2H]C([2H])=C([2H])C(=O)N1CCN(C2CN(c3cc(N4CCC(c5c(C)cnn5C5COC5)CC4)nc(C(F)(F)F)n3)C2)CC1C]

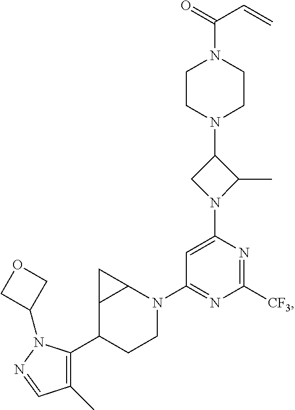 [SMILES: C=CC(=O)N1CCN(C2CN(c3cc(N4CCC(c5c(C)cnn5C5COC5)C5CC54)nc(C(F)(F)F)n3)C2C)CC1]

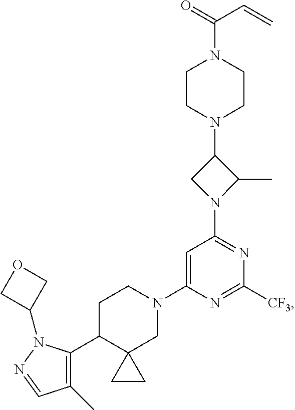 [SMILES: C=CC(=O)N1CCN(C2CN(c3cc(N4CCC(c5c(C)cnn5C5COC5)C5(CC5)C4)nc(C(F)(F)F)n3)C2C)CC1]

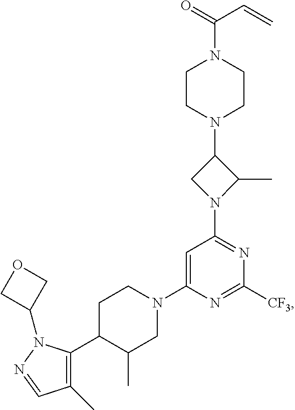 [SMILES: C=CC(=O)N1CCN(C2CN(c3cc(N4CCC(c5c(C)cnn5C5COC5)C(C)C4)nc(C(F)(F)F)n3)C2C)CC1]

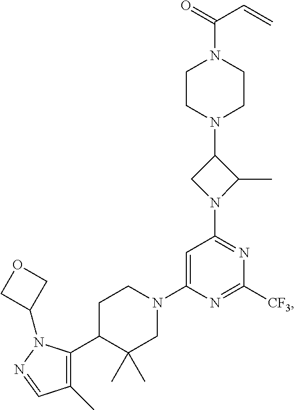 [SMILES: C=CC(=O)N1CCN(C2CN(c3cc(N4CCC(c5c(C)cnn5C5COC5)C(C)(C)C4)nc(C(F)(F)F)n3)C2C)CC1]

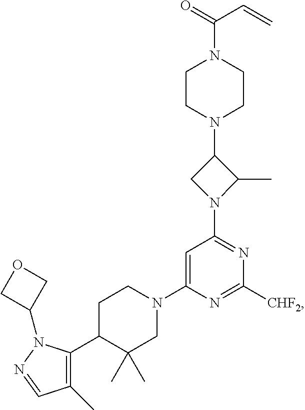 [SMILES: C=CC(=O)N1CCN(C2CN(c3cc(N4CCC(c5c(C)cnn5C5COC5)C(C)(C)C4)nc(C(F)F)n3)C2C)CC1]

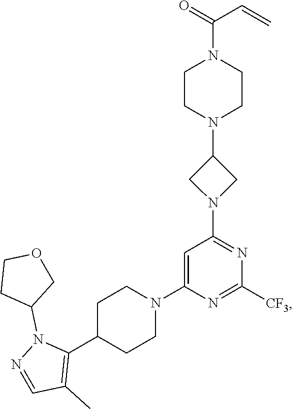 [SMILES: C=CC(=O)N1CCN(C2CN(c3cc(N4CCC(c5c(C)cnn5C5CCOC5)CC4)nc(C(F)(F)F)n3)C2)CC1]

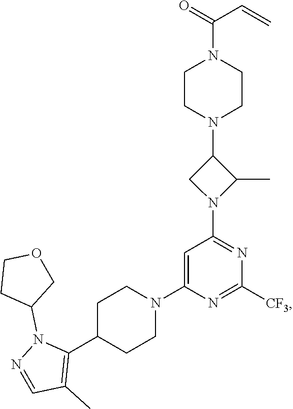 [SMILES: C=CC(=O)N1CCN(C2CN(c3cc(N4CCC(c5c(C)cnn5C5CCOC5)CC4)nc(C(F)(F)F)n3)C2C)CC1]

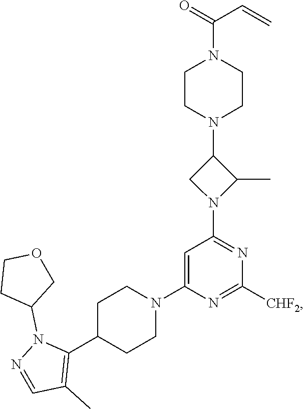 [SMILES: C=CC(=O)N1CCN(C2CN(c3cc(N4CCC(c5c(C)cnn5C5CCOC5)CC4)nc(C(F)F)n3)C2C)CC1]

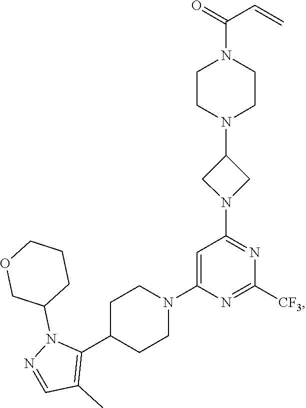 [SMILES: C=CC(=O)N1CCN(C2CN(c3cc(N4CCC(c5c(C)cnn5C5CCCOC5)CC4)nc(C(F)(F)F)n3)C2)CC1]

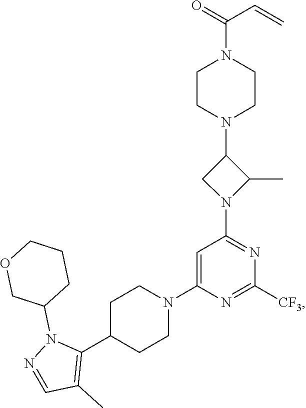 [SMILES: C=CC(=O)N1CCN(C2CN(c3cc(N4CCC(c5c(C)cnn5C5CCCOC5)CC4)nc(C(F)(F)F)n3)C2C)CC1]

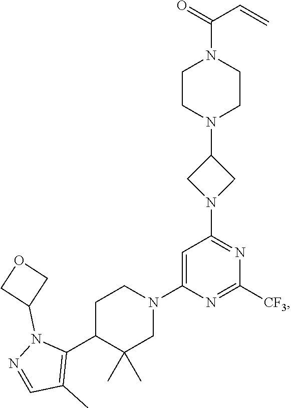 [SMILES: C=CC(=O)N1CCN(C2CN(c3cc(N4CCC(c5c(C)cnn5C5COC5)C(C)(C)C4)nc(C(F)(F)F)n3)C2)CC1]

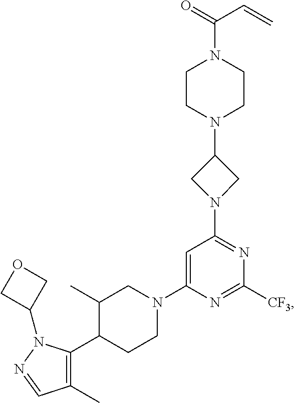 [SMILES: C=CC(=O)N1CCN(C2CN(c3cc(N4CCC(c5c(C)cnn5C5COC5)C(C)C4)nc(C(F)(F)F)n3)C2)CC1]

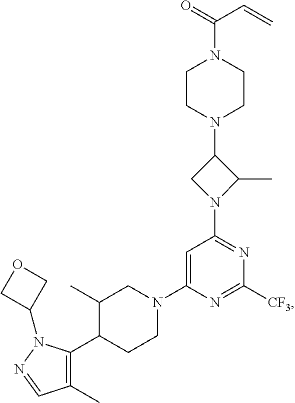 [SMILES: C=CC(=O)N1CCN(C2CN(c3cc(N4CCC(c5c(C)cnn5C5COC5)C(C)C4)nc(C(F)(F)F)n3)C2C)CC1]

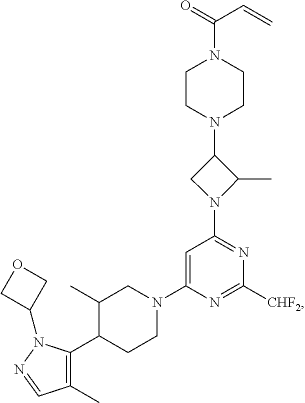 [SMILES: C=CC(=O)N1CCN(C2CN(c3cc(N4CCC(c5c(C)cnn5C5COC5)C(C)C4)nc(C(F)F)n3)C2C)CC1]

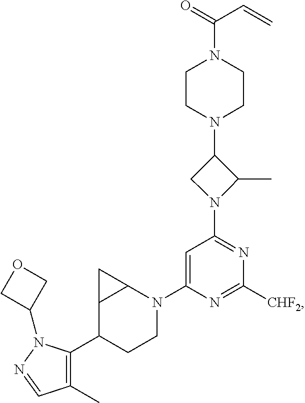 [SMILES: C=CC(=O)N1CCN(C2CN(c3cc(N4CCC(c5c(C)cnn5C5COC5)C5CC54)nc(C(F)F)n3)C2C)CC1]

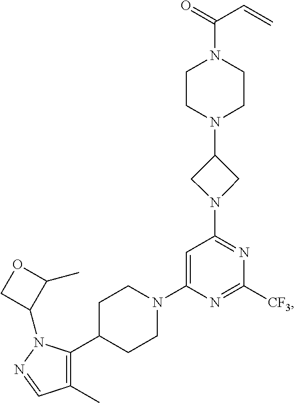 [SMILES: C=CC(=O)N1CCN(C2CN(c3cc(N4CCC(c5c(C)cnn5C5COC5C)CC4)nc(C(F)(F)F)n3)C2)CC1]

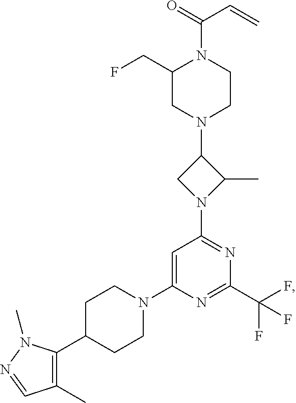 [SMILES: C=CC(=O)N1CCN(C2CN(c3cc(N4CCC(c5c(C)cnn5C)CC4)nc(C(F)(F)F)n3)C2C)CC1CF]

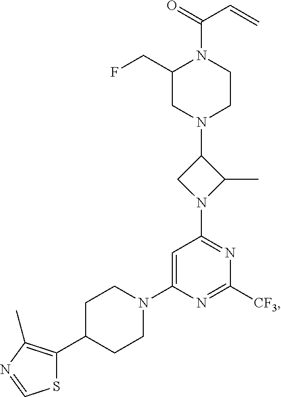 [SMILES: C=CC(=O)N1CCN(C2CN(c3cc(N4CCC(c5scnc5C)CC4)nc(C(F)(F)F)n3)C2C)CC1CF]

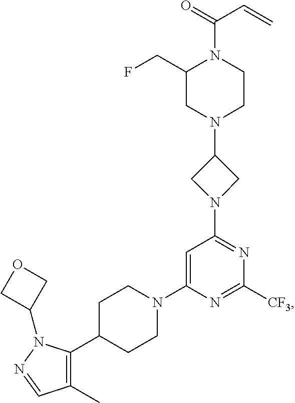 [SMILES: C=CC(=O)N1CCN(C2CN(c3cc(N4CCC(c5c(C)cnn5C5COC5)CC4)nc(C(F)(F)F)n3)C2)CC1CF]

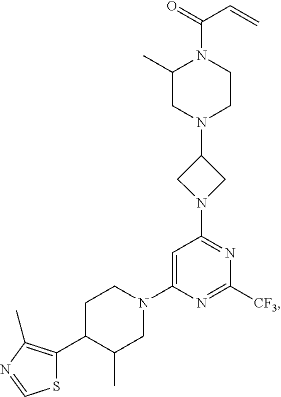 [SMILES: C=CC(=O)N1CCN(C2CN(c3cc(N4CCC(c5scnc5C)C(C)C4)nc(C(F)(F)F)n3)C2)CC1C]

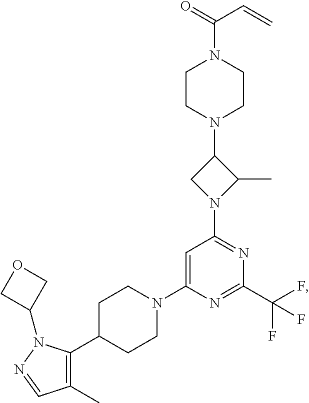 [SMILES: C=CC(=O)N1CCN(C2CN(c3cc(N4CCC(c5c(C)cnn5C5COC5)CC4)nc(C(F)(F)F)n3)C2C)CC1]

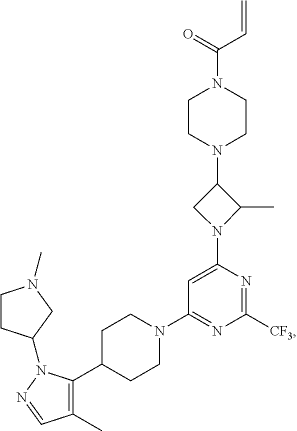 [SMILES: C=CC(=O)N1CCN(C2CN(c3cc(N4CCC(c5c(C)cnn5C5CCN(C)C5)CC4)nc(C(F)(F)F)n3)C2C)CC1]

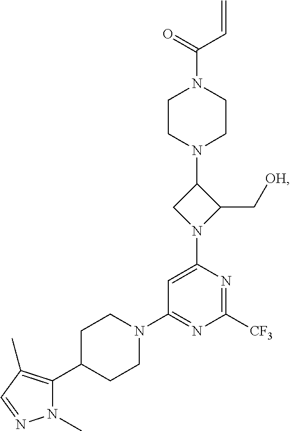 [SMILES: C=CC(=O)N1CCN(C2CN(c3cc(N4CCC(c5c(C)cnn5C)CC4)nc(C(F)(F)F)n3)C2CO)CC1]

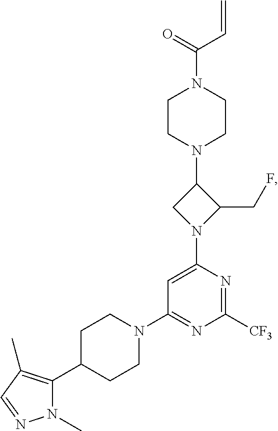 [SMILES: C=CC(=O)N1CCN(C2CN(c3cc(N4CCC(c5c(C)cnn5C)CC4)nc(C(F)(F)F)n3)C2CF)CC1]

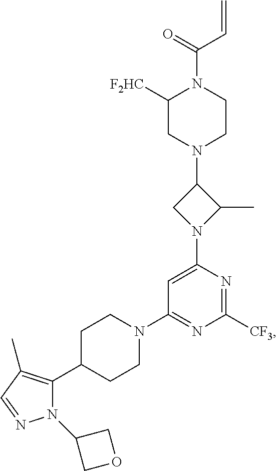 [SMILES: C=CC(=O)N1CCN(C2CN(c3cc(N4CCC(c5c(C)cnn5C5COC5)CC4)nc(C(F)(F)F)n3)C2C)CC1C(F)F]

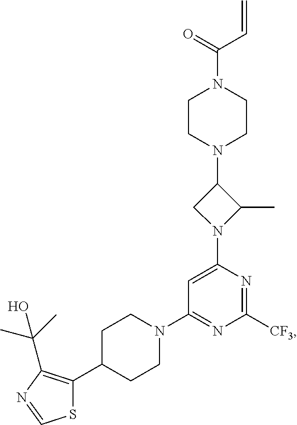 [SMILES: C=CC(=O)N1CCN(C2CN(c3cc(N4CCC(c5scnc5C(C)(C)O)CC4)nc(C(F)(F)F)n3)C2C)CC1]

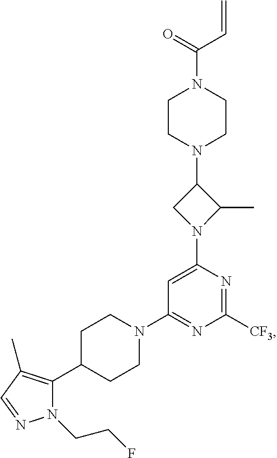 [SMILES: C=CC(=O)N1CCN(C2CN(c3cc(N4CCC(c5c(C)cnn5CCF)CC4)nc(C(F)(F)F)n3)C2C)CC1]

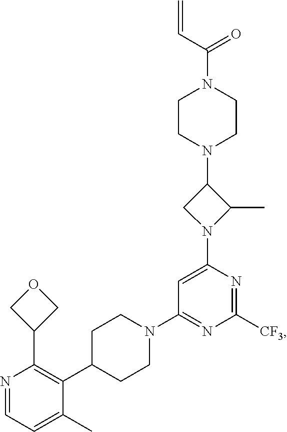 [SMILES: C=CC(=O)N1CCN(C2CN(c3cc(N4CCC(c5c(C)ccnc5C5COC5)CC4)nc(C(F)(F)F)n3)C2C)CC1]

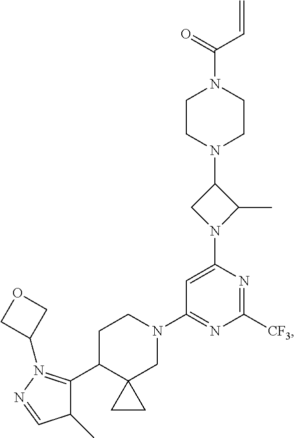

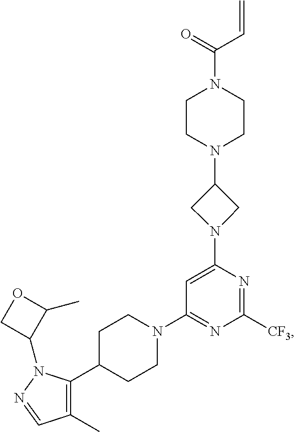 [SMILES: C=CC(=O)N1CCN(C2CN(c3cc(N4CCC(c5c(C)cnn5C5COC5C)CC4)nc(C(F)(F)F)n3)C2)CC1]

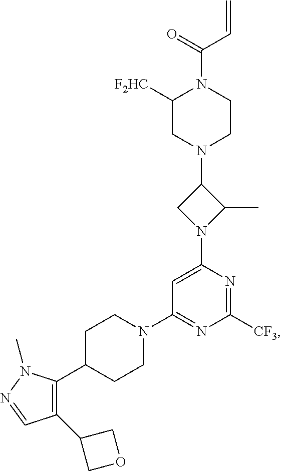 [SMILES: C=CC(=O)N1CCN(C2CN(c3cc(N4CCC(c5c(C6COC6)cnn5C)CC4)nc(C(F)(F)F)n3)C2C)CC1C(F)F]

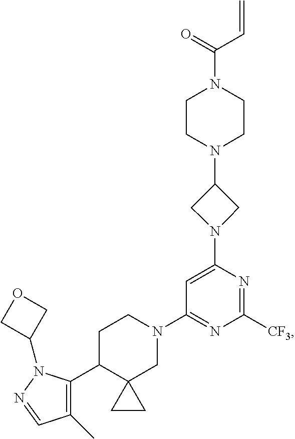 [SMILES: C=CC(=O)N1CCN(C2CN(c3cc(N4CCC(c5c(C)cnn5C5COC5)C5(CC5)C4)nc(C(F)(F)F)n3)C2)CC1]

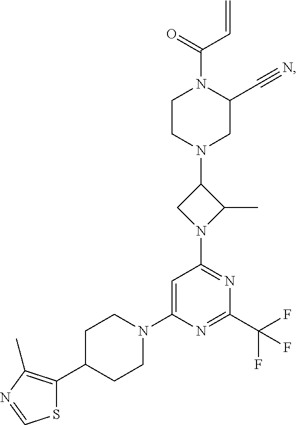 [SMILES: C=CC(=O)N1CCN(C2CN(c3cc(N4CCC(c5scnc5C)CC4)nc(C(F)(F)F)n3)C2C)CC1C#N]

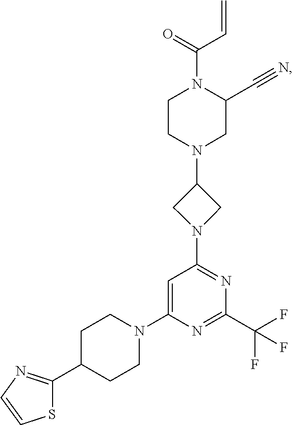 [SMILES: C=CC(=O)N1CCN(C2CN(c3cc(N4CCC(c5nccs5)CC4)nc(C(F)(F)F)n3)C2)CC1C#N]

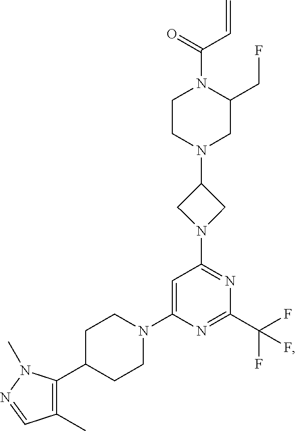 [SMILES: C=CC(=O)N1CCN(C2CN(c3cc(N4CCC(c5c(C)cnn5C)CC4)nc(C(F)(F)F)n3)C2)CC1CF]

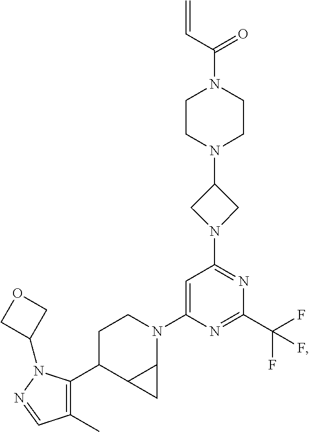 [SMILES: C=CC(=O)N1CCN(C2CN(c3cc(N4CCC(c5c(C)cnn5C5COC5)C5CC54)nc(C(F)(F)F)n3)C2)CC1]

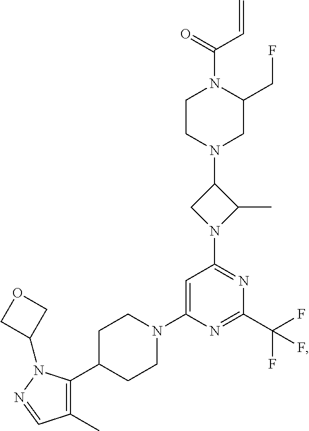 [SMILES: C=CC(=O)N1CCN(C2CN(c3cc(N4CCC(c5c(C)cnn5C5COC5)CC4)nc(C(F)(F)F)n3)C2C)CC1CF]

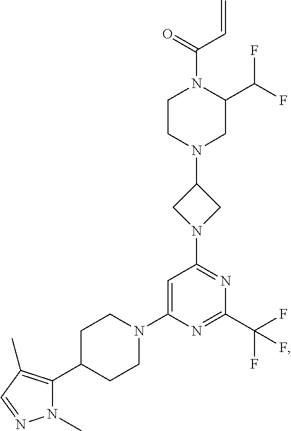 [SMILES: C=CC(=O)N1CCN(C2CN(c3cc(N4CCC(c5c(C)cnn5C)CC4)nc(C(F)(F)F)n3)C2)CC1C(F)F]

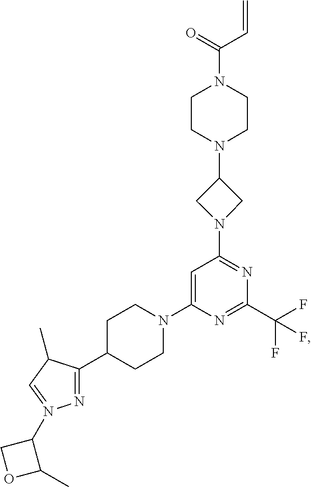

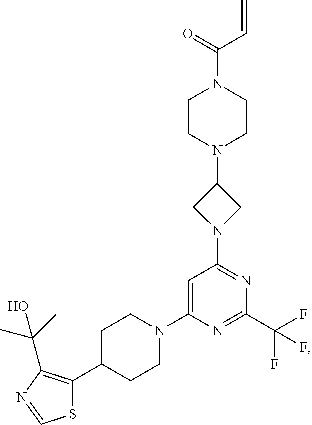 [SMILES: C=CC(=O)N1CCN(C2CN(c3cc(N4CCC(c5scnc5C(C)(C)O)CC4)nc(C(F)(F)F)n3)C2)CC1]

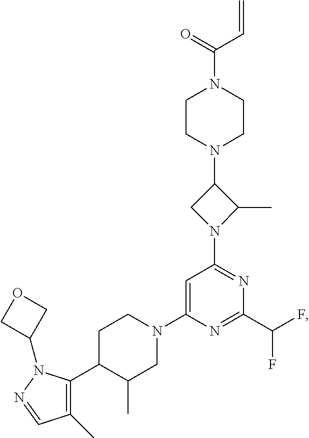 [SMILES: C=CC(=O)N1CCN(C2CN(c3cc(N4CCC(c5c(C)cnn5C5COC5)C(C)C4)nc(C(F)F)n3)C2C)CC1]

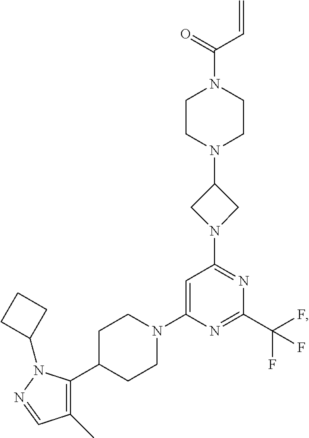 [SMILES: C=CC(=O)N1CCN(C2CN(c3cc(N4CCC(c5c(C)cnn5C5CCC5)CC4)nc(C(F)(F)F)n3)C2)CC1]

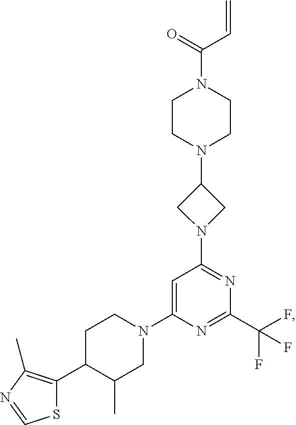 [SMILES: C=CC(=O)N1CCN(C2CN(c3cc(N4CCC(c5scnc5C)C(C)C4)nc(C(F)(F)F)n3)C2)CC1]

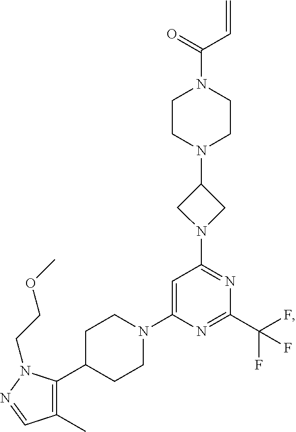 [SMILES: C=CC(=O)N1CCN(C2CN(c3cc(N4CCC(c5c(C)cnn5CCOC)CC4)nc(C(F)(F)F)n3)C2)CC1]

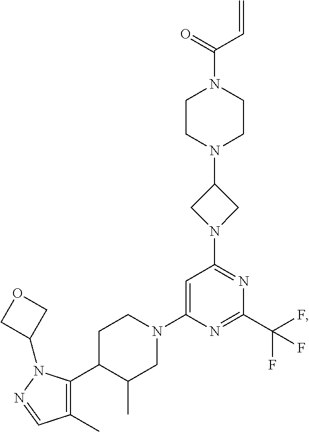 [SMILES: C=CC(=O)N1CCN(C2CN(c3cc(N4CCC(c5c(C)cnn5C5COC5)C(C)C4)nc(C(F)(F)F)n3)C2)CC1]

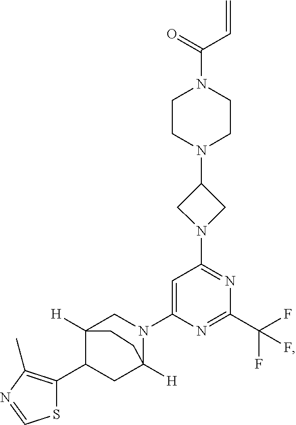 [SMILES: C=CC(=O)N1CCN(C2CN(c3cc(N4CC5CCC4CC5c4scnc4C)nc(C(F)(F)F)n3)C2)CC1]

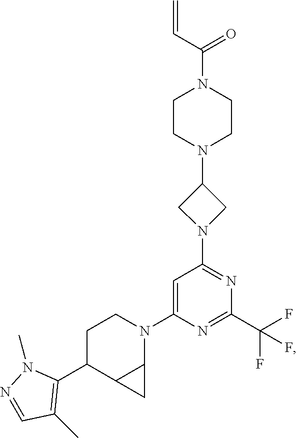 [SMILES: C=CC(=O)N1CCN(C2CN(c3cc(N4CCC(c5c(C)cnn5C)C5CC54)nc(C(F)(F)F)n3)C2)CC1]

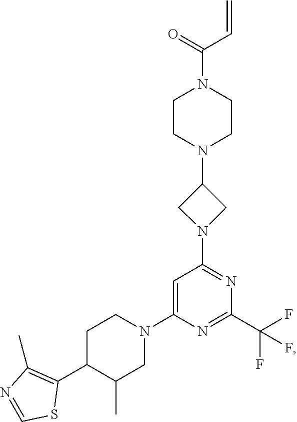 [SMILES: C=CC(=O)N1CCN(C2CN(c3cc(N4CCC(c5scnc5C)C(C)C4)nc(C(F)(F)F)n3)C2)CC1]

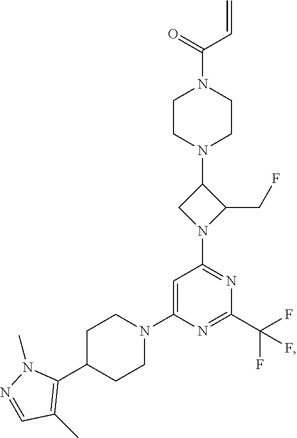 [SMILES: C=CC(=O)N1CCN(C2CN(c3cc(N4CCC(c5c(C)cnn5C)CC4)nc(C(F)(F)F)n3)C2CF)CC1]

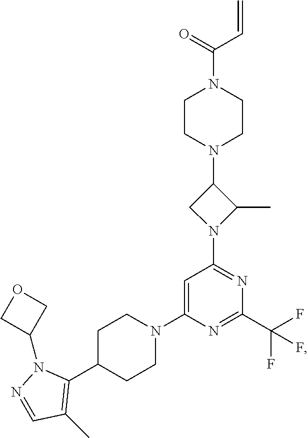 [SMILES: C=CC(=O)N1CCN(C2CN(c3cc(N4CCC(c5c(C)cnn5C5COC5)CC4)nc(C(F)(F)F)n3)C2C)CC1]

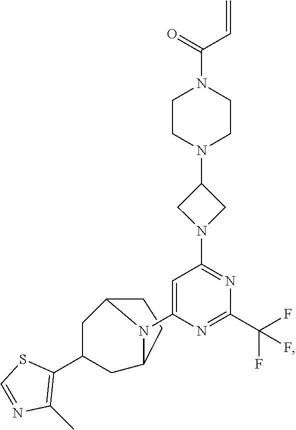 [SMILES: C=CC(=O)N1CCN(C2CN(c3cc(N4C5CCC4CC(c4scnc4C)C5)nc(C(F)(F)F)n3)C2)CC1]

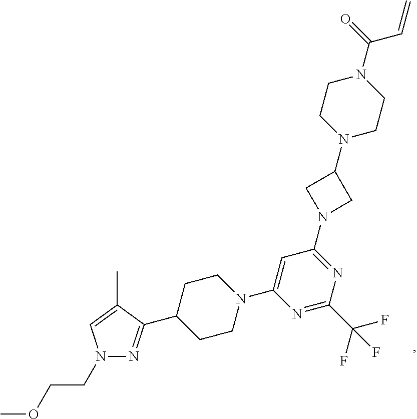 [SMILES: C=CC(=O)N1CCN(C2CN(c3cc(N4CCC(c5nn(CCOC)cc5C)CC4)nc(C(F)(F)F)n3)C2)CC1]

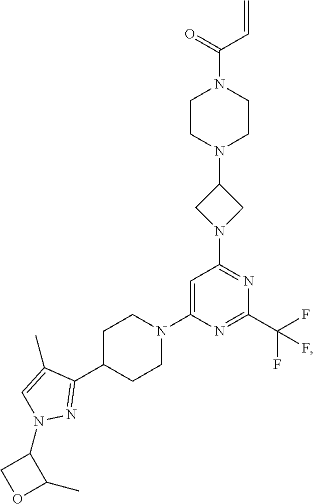 [SMILES: C=CC(=O)N1CCN(C2CN(c3cc(N4CCC(c5nn(C6COC6C)cc5C)CC4)nc(C(F)(F)F)n3)C2)CC1]

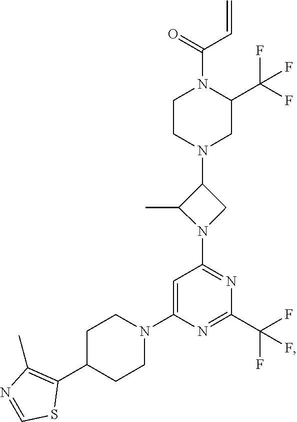 [SMILES: C=CC(=O)N1CCN(C2CN(c3cc(N4CCC(c5scnc5C)CC4)nc(C(F)(F)F)n3)C2C)CC1C(F)(F)F]

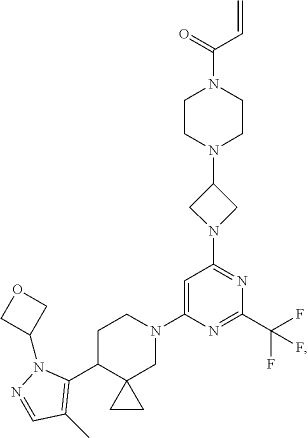 [SMILES: C=CC(=O)N1CCN(C2CN(c3cc(N4CCC(c5c(C)cnn5C5COC5)C5(CC5)C4)nc(C(F)(F)F)n3)C2)CC1]

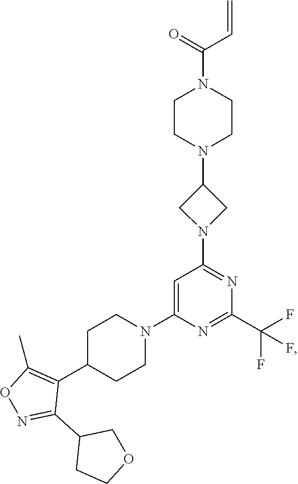 [SMILES: C=CC(=O)N1CCN(C2CN(c3cc(N4CCC(c5c(C6CCOC6)noc5C)CC4)nc(C(F)(F)F)n3)C2)CC1]

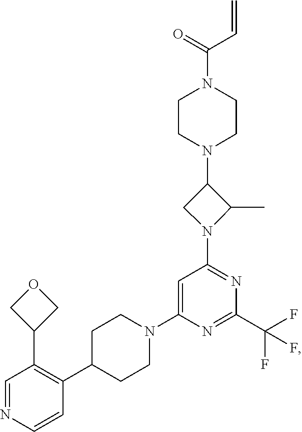 [SMILES: C=CC(=O)N1CCN(C2CN(c3cc(N4CCC(c5ccncc5C5COC5)CC4)nc(C(F)(F)F)n3)C2C)CC1]

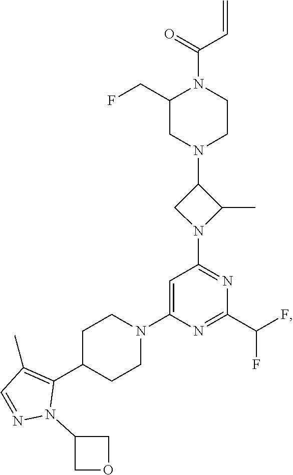 [SMILES: C=CC(=O)N1CCN(C2CN(c3cc(N4CCC(c5c(C)cnn5C5COC5)CC4)nc(C(F)F)n3)C2C)CC1CF]

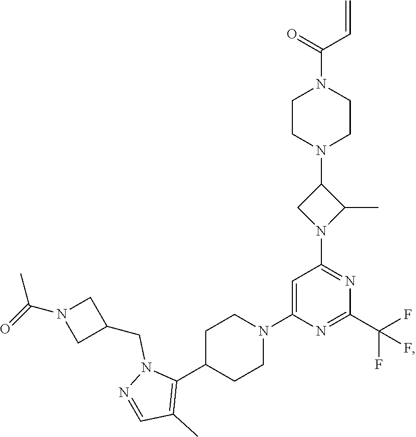 [SMILES: C=CC(=O)N1CCN(C2CN(c3cc(N4CCC(c5c(C)cnn5CC5CN(C(C)=O)C5)CC4)nc(C(F)(F)F)n3)C2C)CC1]

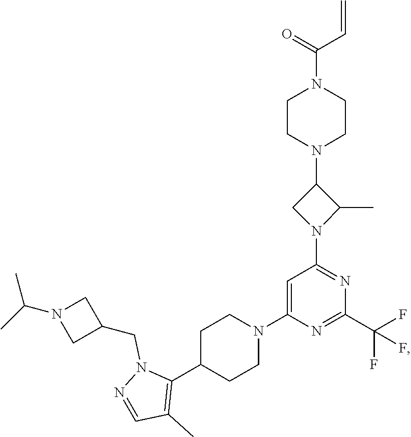 [SMILES: C=CC(=O)N1CCN(C2CN(c3cc(N4CCC(c5c(C)cnn5CC5CN(C(C)C)C5)CC4)nc(C(F)(F)F)n3)C2C)CC1]

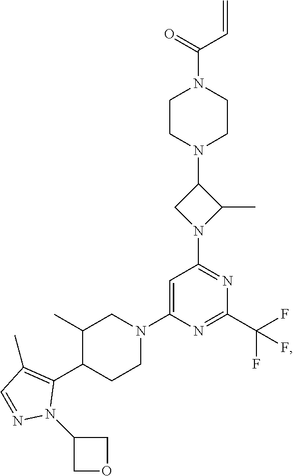 [SMILES: C=CC(=O)N1CCN(C2CN(c3cc(N4CCC(c5c(C)cnn5C5COC5)C(C)C4)nc(C(F)(F)F)n3)C2C)CC1]

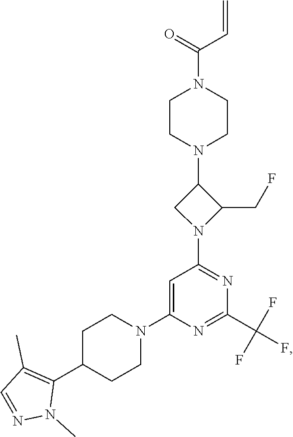 [SMILES: C=CC(=O)N1CCN(C2CN(c3cc(N4CCC(c5c(C)cnn5C)CC4)nc(C(F)(F)F)n3)C2CF)CC1]

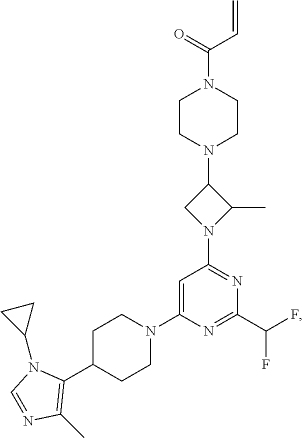 [SMILES: C=CC(=O)N1CCN(C2CN(c3cc(N4CCC(c5c(C)ncn5C5CC5)CC4)nc(C(F)F)n3)C2C)CC1]

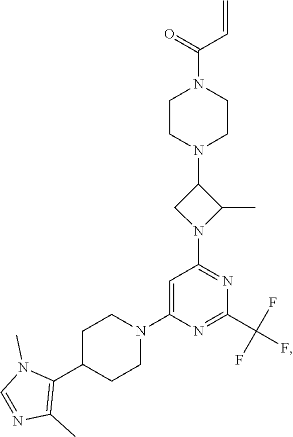 [SMILES: C=CC(=O)N1CCN(C2CN(c3cc(N4CCC(c5c(C)ncn5C)CC4)nc(C(F)(F)F)n3)C2C)CC1]

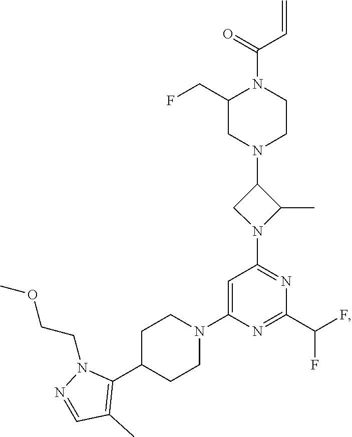 [SMILES: C=CC(=O)N1CCN(C2CN(c3cc(N4CCC(c5c(C)cnn5CCOC)CC4)nc(C(F)F)n3)C2C)CC1CF]

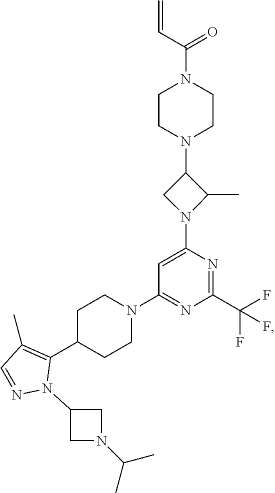 [SMILES: C=CC(=O)N1CCN(C2CN(c3cc(N4CCC(c5c(C)cnn5C5CN(C(C)C)C5)CC4)nc(C(F)(F)F)n3)C2C)CC1]

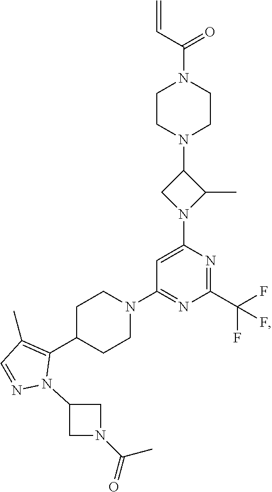 [SMILES: C=CC(=O)N1CCN(C2CN(c3cc(N4CCC(c5c(C)cnn5C5CN(C(C)=O)C5)CC4)nc(C(F)(F)F)n3)C2C)CC1]

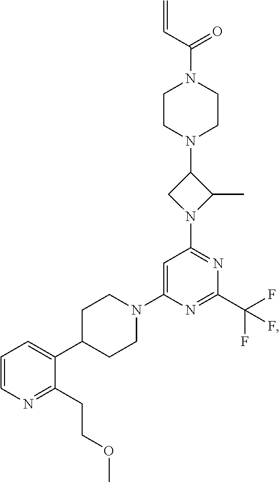 [SMILES: C=CC(=O)N1CCN(C2CN(c3cc(N4CCC(c5cccnc5CCOC)CC4)nc(C(F)(F)F)n3)C2C)CC1]

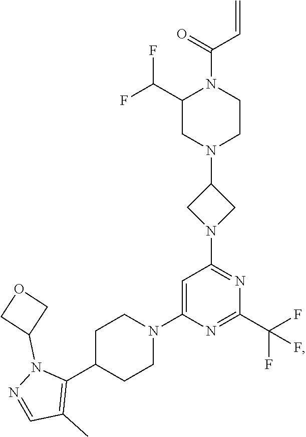 [SMILES: C=CC(=O)N1CCN(C2CN(c3cc(N4CCC(c5c(C)cnn5C5COC5)CC4)nc(C(F)(F)F)n3)C2)CC1C(F)F]

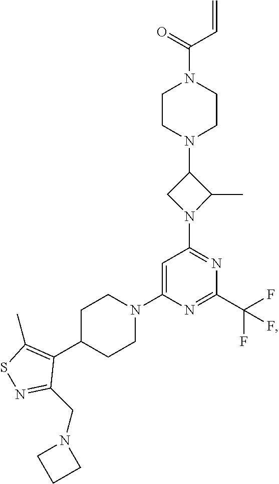 [SMILES: C=CC(=O)N1CCN(C2CN(c3cc(N4CCC(c5c(CN6CCC6)nsc5C)CC4)nc(C(F)(F)F)n3)C2C)CC1]

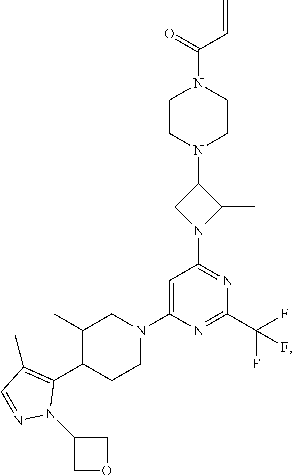 [SMILES: C=CC(=O)N1CCN(C2CN(c3cc(N4CCC(c5c(C)cnn5C5COC5)C(C)C4)nc(C(F)(F)F)n3)C2C)CC1]

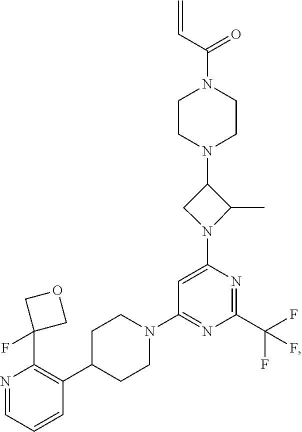 [SMILES: C=CC(=O)N1CCN(C2CN(c3cc(N4CCC(c5cccnc5C5(F)COC5)CC4)nc(C(F)(F)F)n3)C2C)CC1]

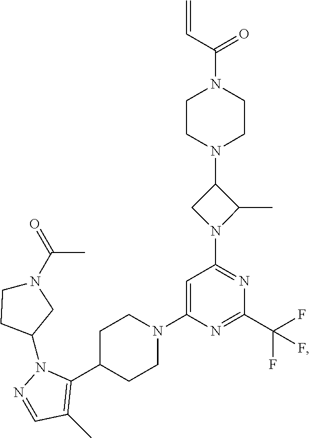 [SMILES: C=CC(=O)N1CCN(C2CN(c3cc(N4CCC(c5c(C)cnn5C5CCN(C(C)=O)C5)CC4)nc(C(F)(F)F)n3)C2C)CC1]

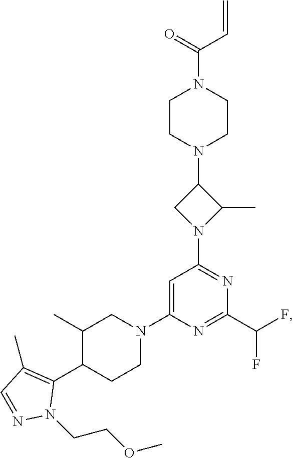 [SMILES: C=CC(=O)N1CCN(C2CN(c3cc(N4CCC(c5c(C)cnn5CCOC)C(C)C4)nc(C(F)F)n3)C2C)CC1]

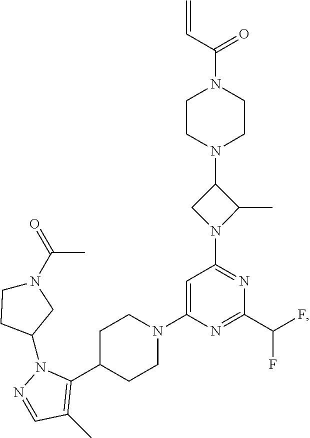 [SMILES: C=CC(=O)N1CCN(C2CN(c3cc(N4CCC(c5c(C)cnn5C5CCN(C(C)=O)C5)CC4)nc(C(F)F)n3)C2C)CC1]

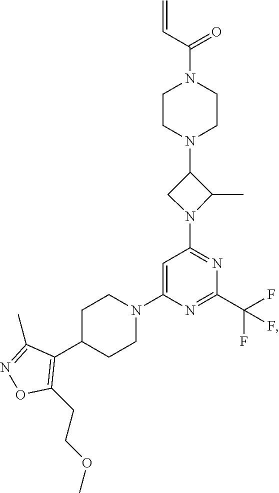 [SMILES: C=CC(=O)N1CCN(C2CN(c3cc(N4CCC(c5c(C)noc5CCOC)CC4)nc(C(F)(F)F)n3)C2C)CC1]

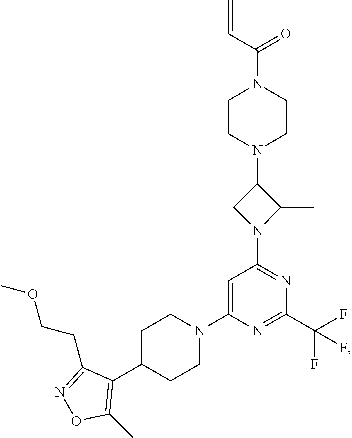 [SMILES: C=CC(=O)N1CCN(C2CN(c3cc(N4CCC(c5c(CCOC)noc5C)CC4)nc(C(F)(F)F)n3)C2C)CC1]

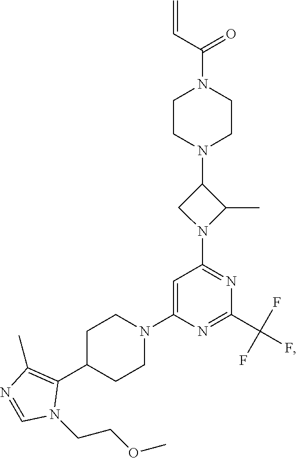 [SMILES: C=CC(=O)N1CCN(C2CN(c3cc(N4CCC(c5c(C)ncn5CCOC)CC4)nc(C(F)(F)F)n3)C2C)CC1]

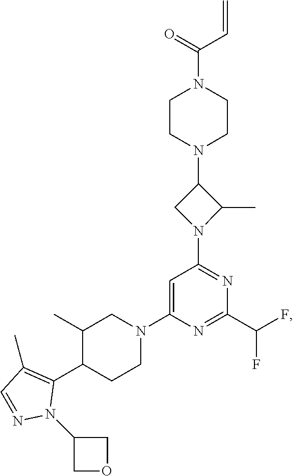 [SMILES: C=CC(=O)N1CCN(C2CN(c3cc(N4CCC(c5c(C)cnn5C5COC5)C(C)C4)nc(C(F)F)n3)C2C)CC1]

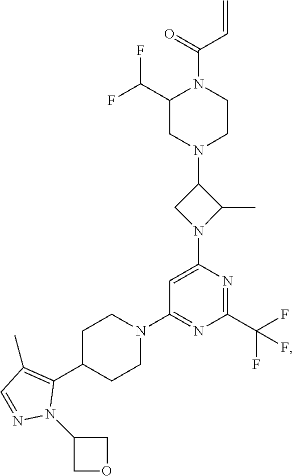 [SMILES: C=CC(=O)N1CCN(C2CN(c3cc(N4CCC(c5c(C)cnn5C5COC5)CC4)nc(C(F)(F)F)n3)C2C)CC1C(F)F]

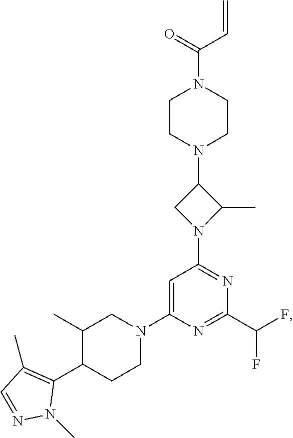 [SMILES: C=CC(=O)N1CCN(C2CN(c3cc(N4CCC(c5c(C)cnn5C)C(C)C4)nc(C(F)F)n3)C2C)CC1]

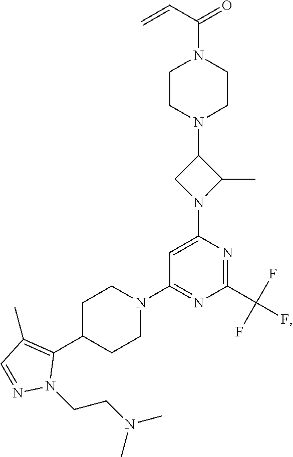 [SMILES: C=CC(=O)N1CCN(C2CN(c3cc(N4CCC(c5c(C)cnn5CCN(C)C)CC4)nc(C(F)(F)F)n3)C2C)CC1]

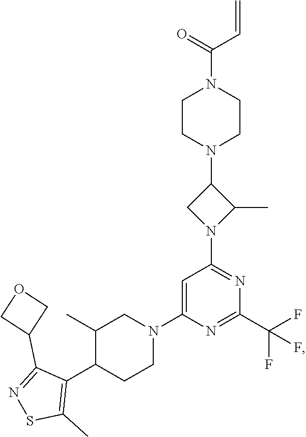 [SMILES: C=CC(=O)N1CCN(C2CN(c3cc(N4CCC(c5c(C6COC6)nsc5C)C(C)C4)nc(C(F)(F)F)n3)C2C)CC1]

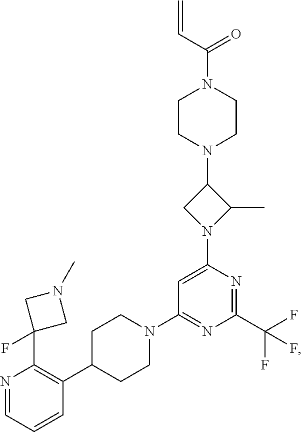 [SMILES: C=CC(=O)N1CCN(C2CN(c3cc(N4CCC(c5cccnc5C5(F)CN(C)C5)CC4)nc(C(F)(F)F)n3)C2C)CC1]

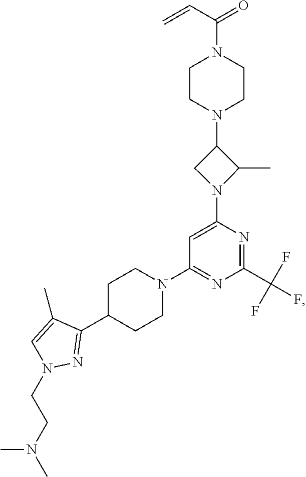 [SMILES: C=CC(=O)N1CCN(C2CN(c3cc(N4CCC(c5nn(CCN(C)C)cc5C)CC4)nc(C(F)(F)F)n3)C2C)CC1]

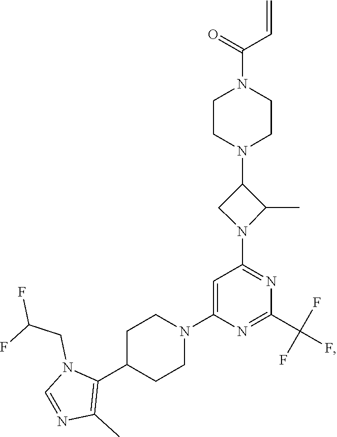 [SMILES: C=CC(=O)N1CCN(C2CN(c3cc(N4CCC(c5c(C)ncn5CC(F)F)CC4)nc(C(F)(F)F)n3)C2C)CC1]

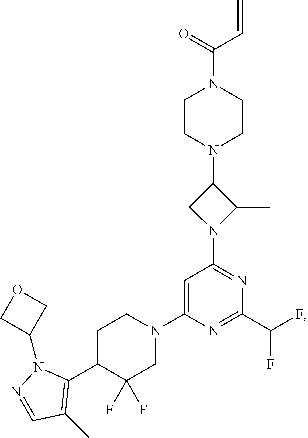 [SMILES: C=CC(=O)N1CCN(C2CN(c3cc(N4CCC(c5c(C)cnn5C5COC5)C(F)(F)C4)nc(C(F)F)n3)C2C)CC1]

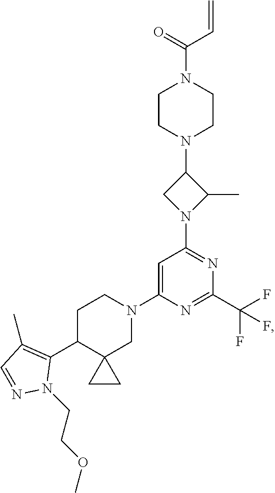 [SMILES: C=CC(=O)N1CCN(C2CN(c3cc(N4CCC(c5c(C)cnn5CCOC)C5(CC5)C4)nc(C(F)(F)F)n3)C2C)CC1]

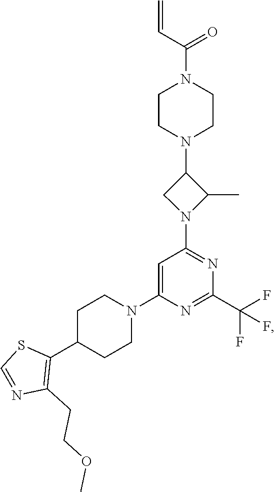 [SMILES: C=CC(=O)N1CCN(C2CN(c3cc(N4CCC(c5scnc5CCOC)CC4)nc(C(F)(F)F)n3)C2C)CC1]

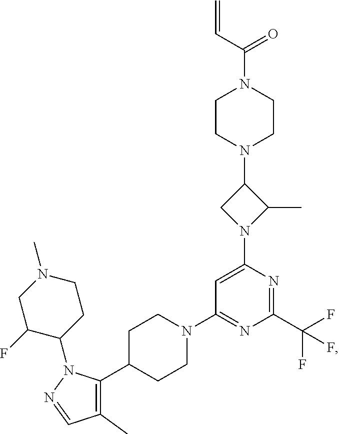 [SMILES: C=CC(=O)N1CCN(C2CN(c3cc(N4CCC(c5c(C)cnn5C5CCN(C)CC5F)CC4)nc(C(F)(F)F)n3)C2C)CC1]

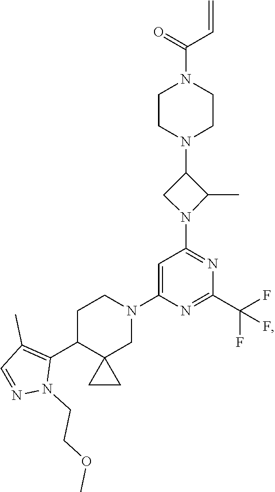 [SMILES: C=CC(=O)N1CCN(C2CN(c3cc(N4CCC(c5c(C)cnn5CCOC)C5(CC5)C4)nc(C(F)(F)F)n3)C2C)CC1]

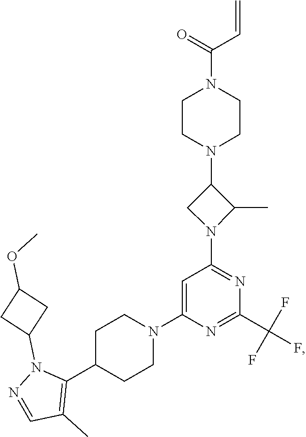 [SMILES: C=CC(=O)N1CCN(C2CN(c3cc(N4CCC(c5c(C)cnn5C5CC(OC)C5)CC4)nc(C(F)(F)F)n3)C2C)CC1]

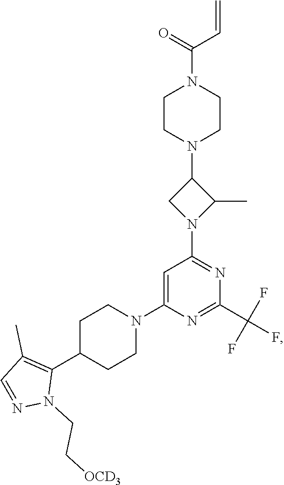

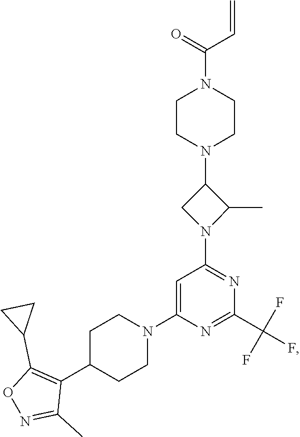 [SMILES: C=CC(=O)N1CCN(C2CN(c3cc(N4CCC(c5c(C)noc5C5CC5)CC4)nc(C(F)(F)F)n3)C2C)CC1]

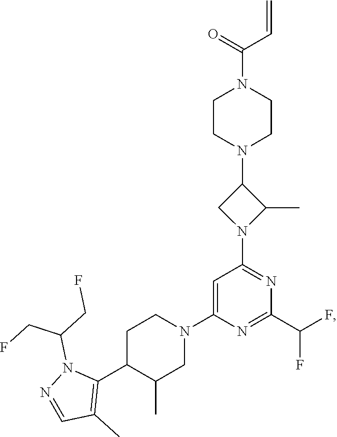 [SMILES: C=CC(=O)N1CCN(C2CN(c3cc(N4CCC(c5c(C)cnn5C(CF)CF)C(C)C4)nc(C(F)F)n3)C2C)CC1]

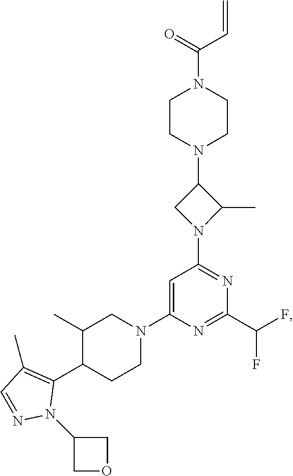 [SMILES: C=CC(=O)N1CCN(C2CN(c3cc(N4CCC(c5c(C)cnn5C5COC5)C(C)C4)nc(C(F)F)n3)C2C)CC1]

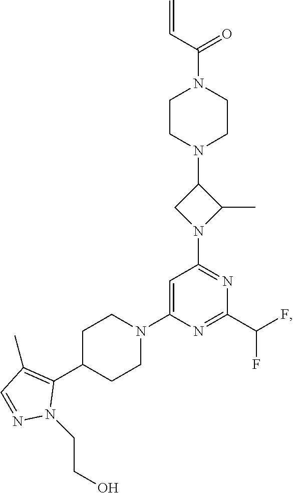 [SMILES: C=CC(=O)N1CCN(C2CN(c3cc(N4CCC(c5c(C)cnn5CCO)CC4)nc(C(F)F)n3)C2C)CC1]

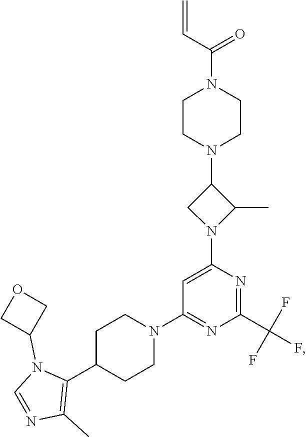 [SMILES: C=CC(=O)N1CCN(C2CN(c3cc(N4CCC(c5c(C)ncn5C5COC5)CC4)nc(C(F)(F)F)n3)C2C)CC1]

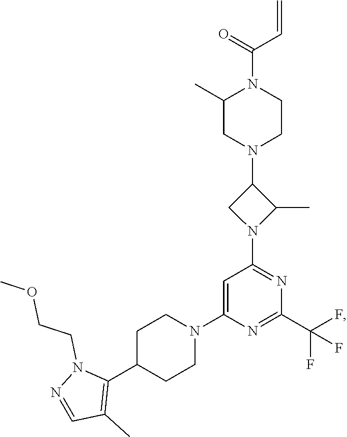 [SMILES: C=CC(=O)N1CCN(C2CN(c3cc(N4CCC(c5c(C)cnn5CCOC)CC4)nc(C(F)(F)F)n3)C2C)CC1C]

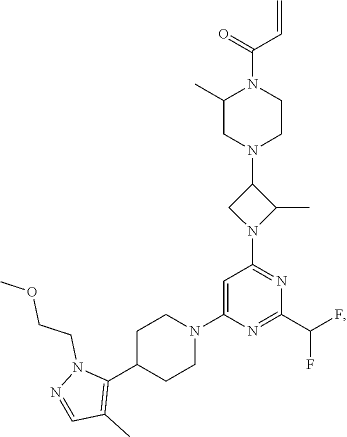 [SMILES: C=CC(=O)N1CCN(C2CN(c3cc(N4CCC(c5c(C)cnn5CCOC)CC4)nc(C(F)F)n3)C2C)CC1C]

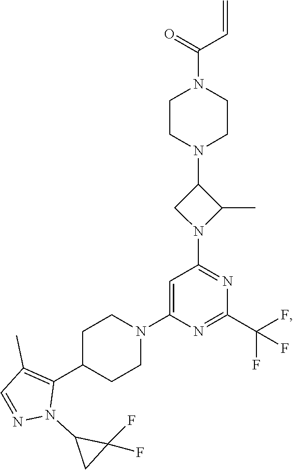 [SMILES: C=CC(=O)N1CCN(C2CN(c3cc(N4CCC(c5c(C)cnn5C5CC5(F)F)CC4)nc(C(F)(F)F)n3)C2C)CC1]

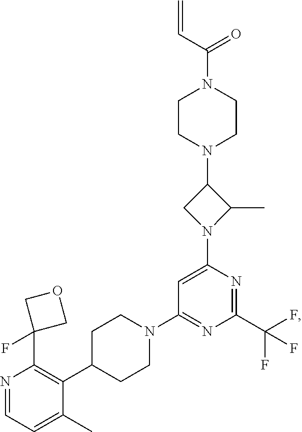 [SMILES: C=CC(=O)N1CCN(C2CN(c3cc(N4CCC(c5c(C)ccnc5C5(F)COC5)CC4)nc(C(F)(F)F)n3)C2C)CC1]

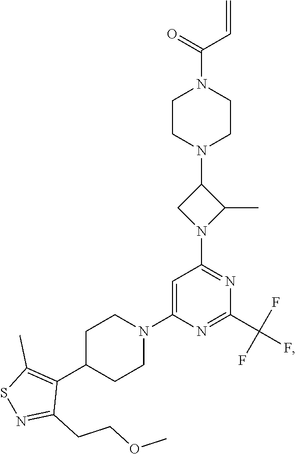 [SMILES: C=CC(=O)N1CCN(C2CN(c3cc(N4CCC(c5c(CCOC)nsc5C)CC4)nc(C(F)(F)F)n3)C2C)CC1]

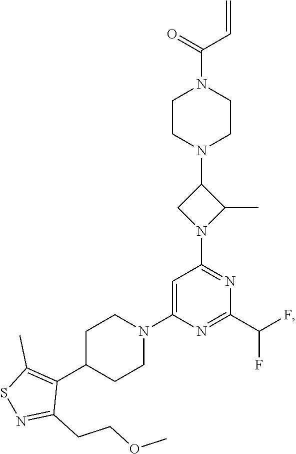 [SMILES: C=CC(=O)N1CCN(C2CN(c3cc(N4CCC(c5c(CCOC)nsc5C)CC4)nc(C(F)F)n3)C2C)CC1]

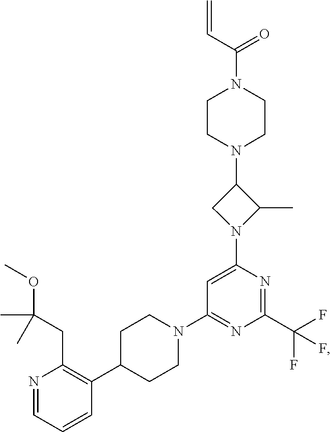 [SMILES: C=CC(=O)N1CCN(C2CN(c3cc(N4CCC(c5cccnc5CC(C)(C)OC)CC4)nc(C(F)(F)F)n3)C2C)CC1]

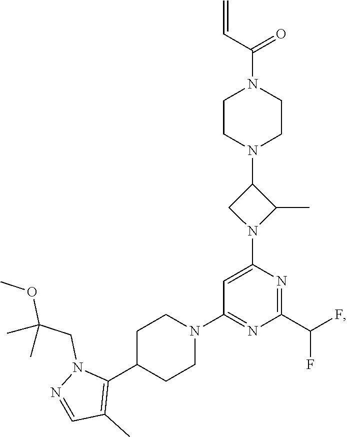 [SMILES: C=CC(=O)N1CCN(C2CN(c3cc(N4CCC(c5c(C)cnn5CC(C)(C)OC)CC4)nc(C(F)F)n3)C2C)CC1]

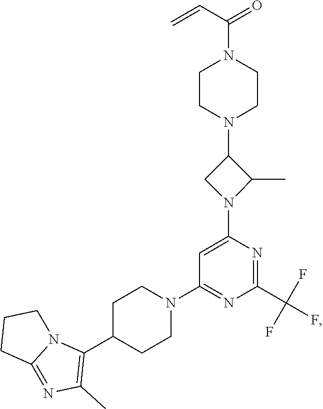 [SMILES: C=CC(=O)N1CCN(C2CN(c3cc(N4CCC(c5c(C)nc6n5CCC6)CC4)nc(C(F)(F)F)n3)C2C)CC1]

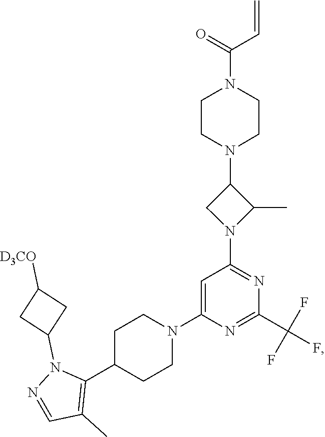

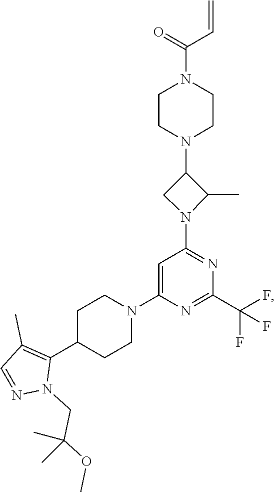 [SMILES: C=CC(=O)N1CCN(C2CN(c3cc(N4CCC(c5c(C)cnn5CC(C)(C)OC)CC4)nc(C(F)(F)F)n3)C2C)CC1]

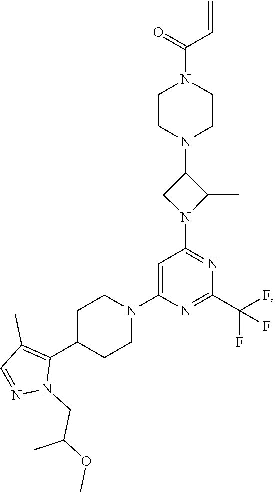 [SMILES: C=CC(=O)N1CCN(C2CN(c3cc(N4CCC(c5c(C)cnn5CC(C)OC)CC4)nc(C(F)(F)F)n3)C2C)CC1]

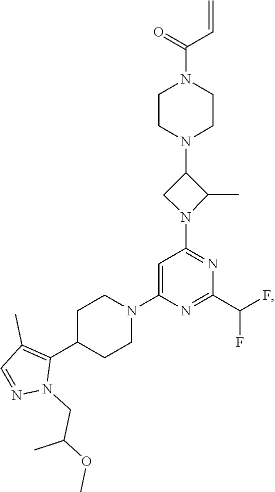 [SMILES: C=CC(=O)N1CCN(C2CN(c3cc(N4CCC(c5c(C)cnn5CC(C)OC)CC4)nc(C(F)F)n3)C2C)CC1]

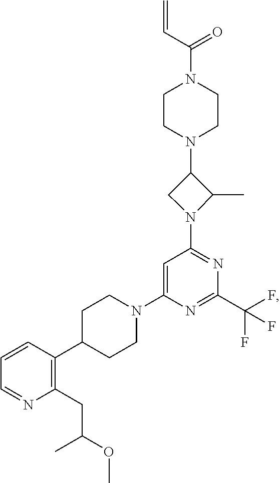 [SMILES: C=CC(=O)N1CCN(C2CN(c3cc(N4CCC(c5cccnc5CC(C)OC)CC4)nc(C(F)(F)F)n3)C2C)CC1]

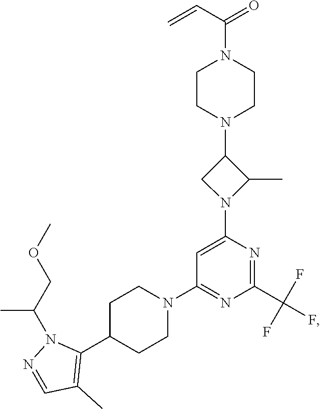 [SMILES: C=CC(=O)N1CCN(C2CN(c3cc(N4CCC(c5c(C)cnn5C(C)COC)CC4)nc(C(F)(F)F)n3)C2C)CC1]

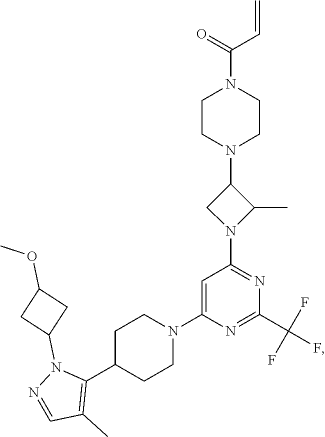 [SMILES: C=CC(=O)N1CCN(C2CN(c3cc(N4CCC(c5c(C)cnn5C5CC(OC)C5)CC4)nc(C(F)(F)F)n3)C2C)CC1]

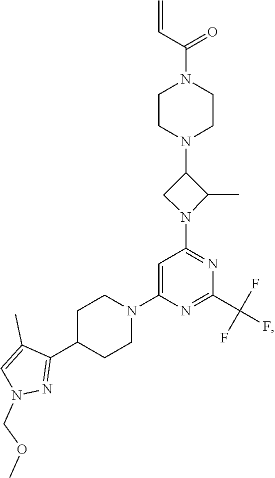 [SMILES: C=CC(=O)N1CCN(C2CN(c3cc(N4CCC(c5nn(COC)cc5C)CC4)nc(C(F)(F)F)n3)C2C)CC1]

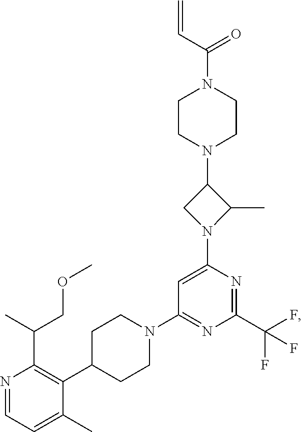 [SMILES: C=CC(=O)N1CCN(C2CN(c3cc(N4CCC(c5c(C)ccnc5C(C)COC)CC4)nc(C(F)(F)F)n3)C2C)CC1]

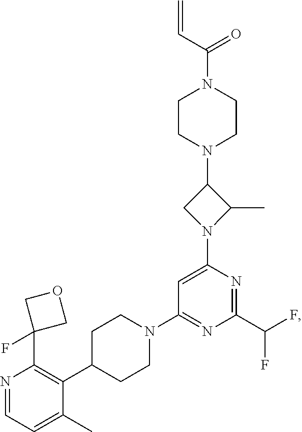 [SMILES: C=CC(=O)N1CCN(C2CN(c3cc(N4CCC(c5c(C)ccnc5C5(F)COC5)CC4)nc(C(F)F)n3)C2C)CC1]

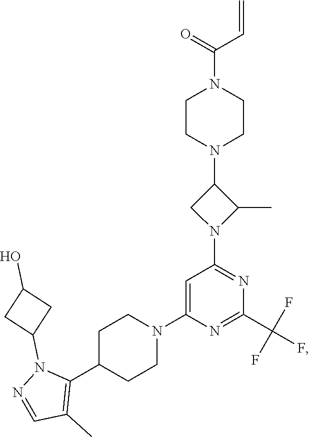 [SMILES: C=CC(=O)N1CCN(C2CN(c3cc(N4CCC(c5c(C)cnn5C5CC(O)C5)CC4)nc(C(F)(F)F)n3)C2C)CC1]

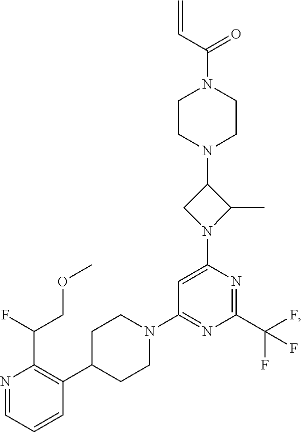 [SMILES: C=CC(=O)N1CCN(C2CN(c3cc(N4CCC(c5cccnc5C(F)COC)CC4)nc(C(F)(F)F)n3)C2C)CC1]

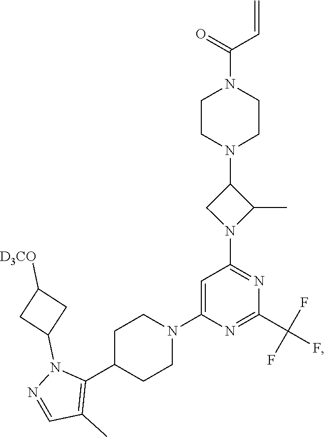

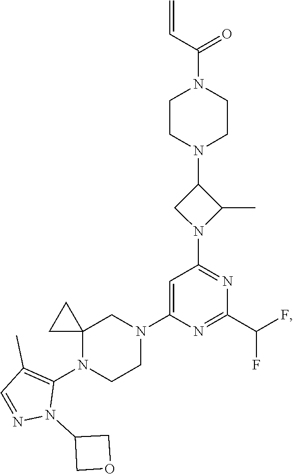 [SMILES: C=CC(=O)N1CCN(C2CN(c3cc(N4CCN(c5c(C)cnn5C5COC5)C5(CC5)C4)nc(C(F)F)n3)C2C)CC1]

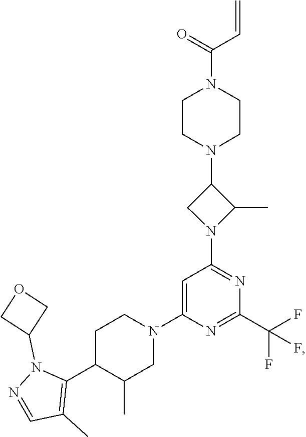 [SMILES: C=CC(=O)N1CCN(C2CN(c3cc(N4CCC(c5c(C)cnn5C5COC5)C(C)C4)nc(C(F)(F)F)n3)C2C)CC1]

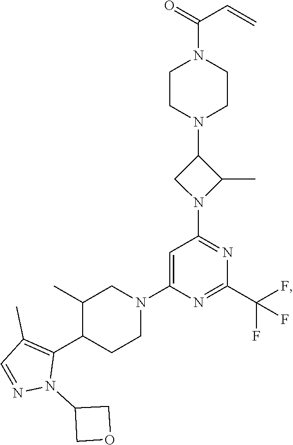 [SMILES: C=CC(=O)N1CCN(C2CN(c3cc(N4CCC(c5c(C)cnn5C5COC5)C(C)C4)nc(C(F)(F)F)n3)C2C)CC1]

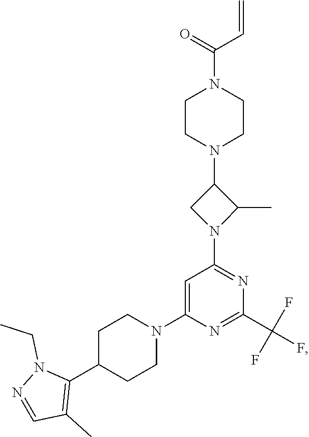 [SMILES: C=CC(=O)N1CCN(C2CN(c3cc(N4CCC(c5c(C)cnn5CC)CC4)nc(C(F)(F)F)n3)C2C)CC1]

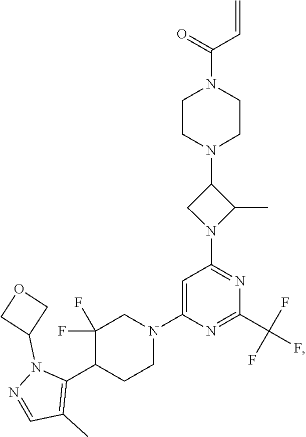 [SMILES: C=CC(=O)N1CCN(C2CN(c3cc(N4CCC(c5c(C)cnn5C5COC5)C(F)(F)C4)nc(C(F)(F)F)n3)C2C)CC1]

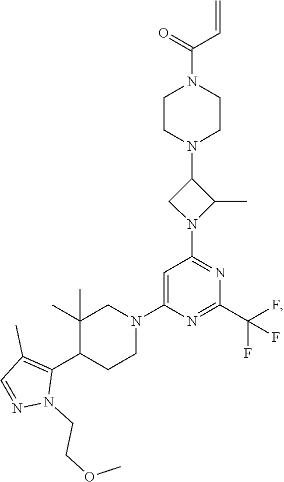 [SMILES: C=CC(=O)N1CCN(C2CN(c3cc(N4CCC(c5c(C)cnn5CCOC)C(C)(C)C4)nc(C(F)(F)F)n3)C2C)CC1]

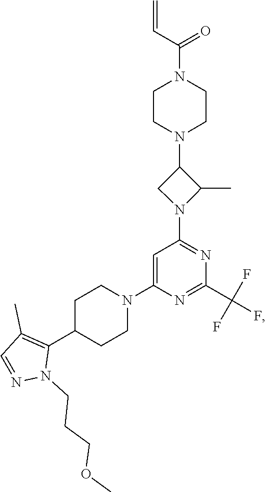 [SMILES: C=CC(=O)N1CCN(C2CN(c3cc(N4CCC(c5c(C)cnn5CCCOC)CC4)nc(C(F)(F)F)n3)C2C)CC1]

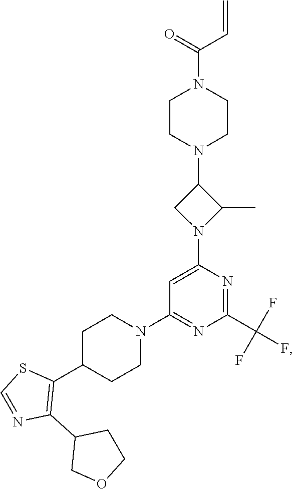 [SMILES: C=CC(=O)N1CCN(C2CN(c3cc(N4CCC(c5scnc5C5CCOC5)CC4)nc(C(F)(F)F)n3)C2C)CC1]

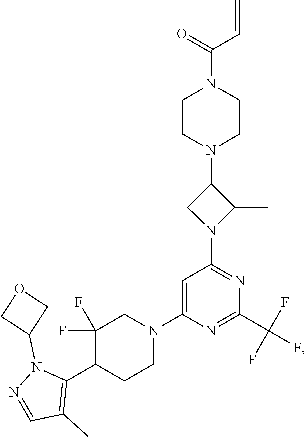 [SMILES: C=CC(=O)N1CCN(C2CN(c3cc(N4CCC(c5c(C)cnn5C5COC5)C(F)(F)C4)nc(C(F)(F)F)n3)C2C)CC1]

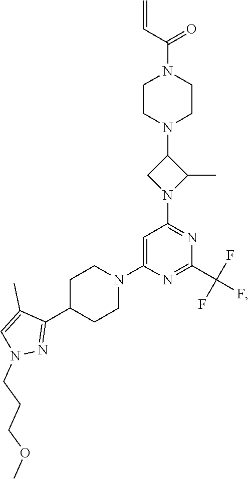 [SMILES: C=CC(=O)N1CCN(C2CN(c3cc(N4CCC(c5nn(CCCOC)cc5C)CC4)nc(C(F)(F)F)n3)C2C)CC1]

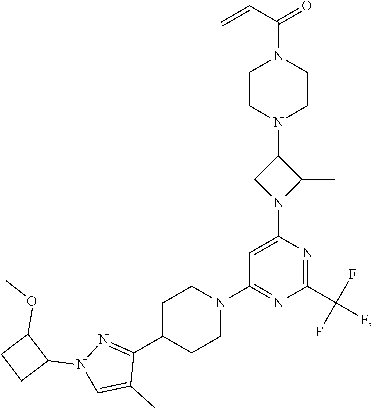 [SMILES: C=CC(=O)N1CCN(C2CN(c3cc(N4CCC(c5nn(C6CCC6OC)cc5C)CC4)nc(C(F)(F)F)n3)C2C)CC1]

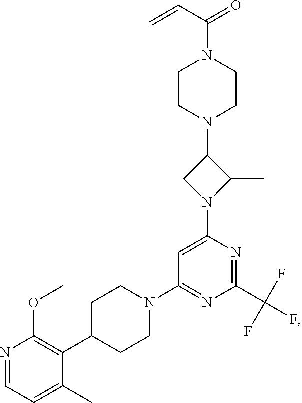 [SMILES: C=CC(=O)N1CCN(C2CN(c3cc(N4CCC(c5c(C)ccnc5OC)CC4)nc(C(F)(F)F)n3)C2C)CC1]

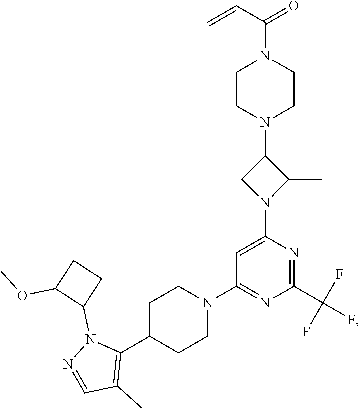 [SMILES: C=CC(=O)N1CCN(C2CN(c3cc(N4CCC(c5c(C)cnn5C5CCC5OC)CC4)nc(C(F)(F)F)n3)C2C)CC1]

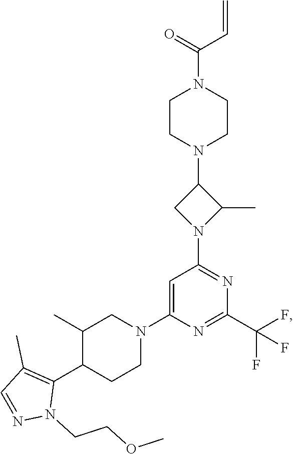 [SMILES: C=CC(=O)N1CCN(C2CN(c3cc(N4CCC(c5c(C)cnn5CCOC)C(C)C4)nc(C(F)(F)F)n3)C2C)CC1]

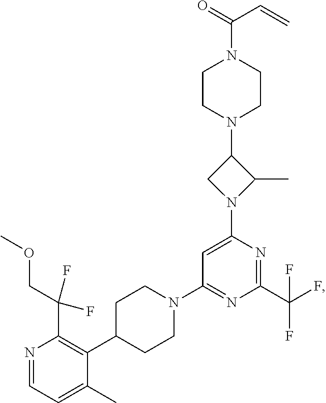 [SMILES: C=CC(=O)N1CCN(C2CN(c3cc(N4CCC(c5c(C)ccnc5C(F)(F)COC)CC4)nc(C(F)(F)F)n3)C2C)CC1]

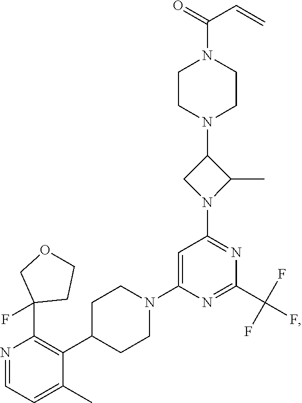 [SMILES: C=CC(=O)N1CCN(C2CN(c3cc(N4CCC(c5c(C)ccnc5C5(F)CCOC5)CC4)nc(C(F)(F)F)n3)C2C)CC1]

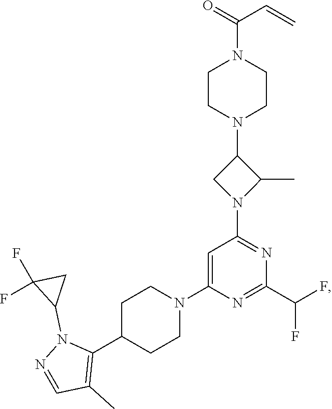 [SMILES: C=CC(=O)N1CCN(C2CN(c3cc(N4CCC(c5c(C)cnn5C5CC5(F)F)CC4)nc(C(F)F)n3)C2C)CC1]

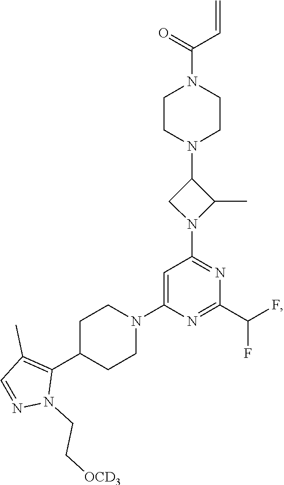

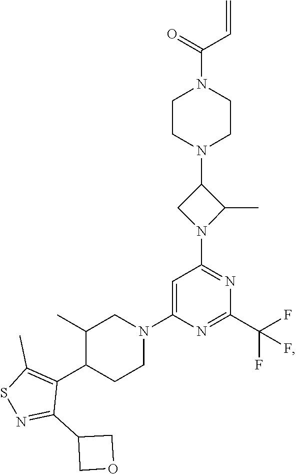 [SMILES: C=CC(=O)N1CCN(C2CN(c3cc(N4CCC(c5c(C6COC6)nsc5C)C(C)C4)nc(C(F)(F)F)n3)C2C)CC1]

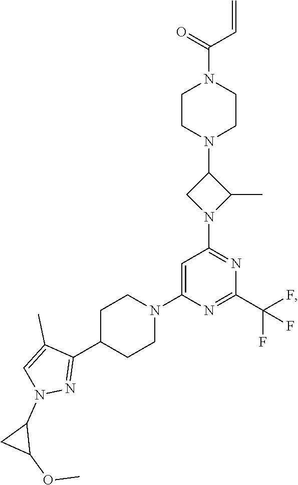 [SMILES: C=CC(=O)N1CCN(C2CN(c3cc(N4CCC(c5nn(C6CC6OC)cc5C)CC4)nc(C(F)(F)F)n3)C2C)CC1]

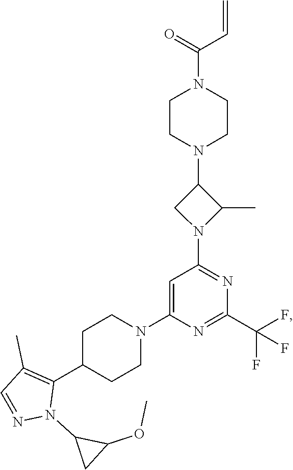 [SMILES: C=CC(=O)N1CCN(C2CN(c3cc(N4CCC(c5c(C)cnn5C5CC5OC)CC4)nc(C(F)(F)F)n3)C2C)CC1]

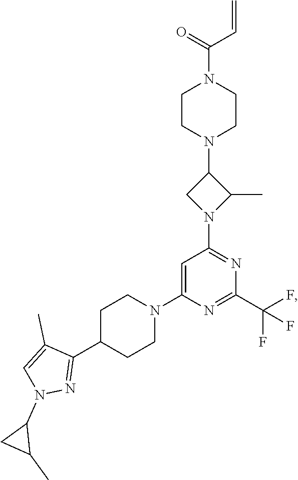 [SMILES: C=CC(=O)N1CCN(C2CN(c3cc(N4CCC(c5nn(C6CC6C)cc5C)CC4)nc(C(F)(F)F)n3)C2C)CC1]

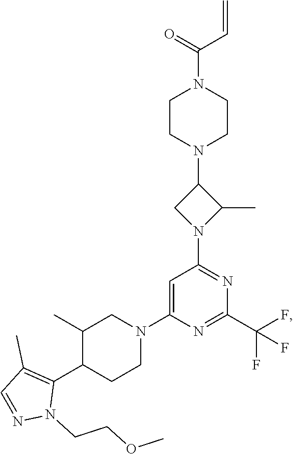 [SMILES: C=CC(=O)N1CCN(C2CN(c3cc(N4CCC(c5c(C)cnn5CCOC)C(C)C4)nc(C(F)(F)F)n3)C2C)CC1]

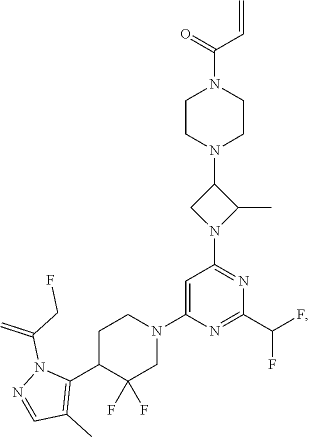 [SMILES: C=CC(=O)N1CCN(C2CN(c3cc(N4CCC(c5c(C)cnn5C(=C)CF)C(F)(F)C4)nc(C(F)F)n3)C2C)CC1]

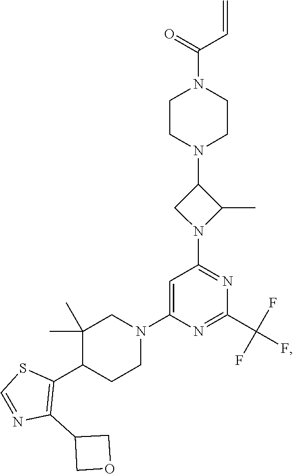 [SMILES: C=CC(=O)N1CCN(C2CN(c3cc(N4CCC(c5scnc5C5COC5)C(C)(C)C4)nc(C(F)(F)F)n3)C2C)CC1]

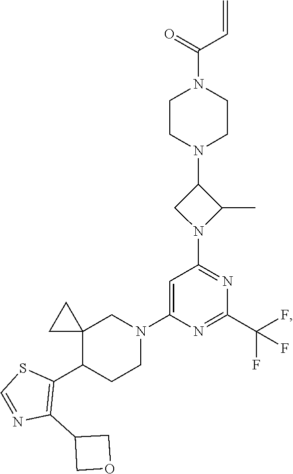 [SMILES: C=CC(=O)N1CCN(C2CN(c3cc(N4CCC(c5scnc5C5COC5)C5(CC5)C4)nc(C(F)(F)F)n3)C2C)CC1]

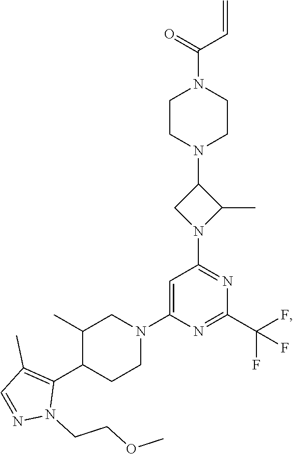 [SMILES: C=CC(=O)N1CCN(C2CN(c3cc(N4CCC(c5c(C)cnn5CCOC)C(C)C4)nc(C(F)(F)F)n3)C2C)CC1]

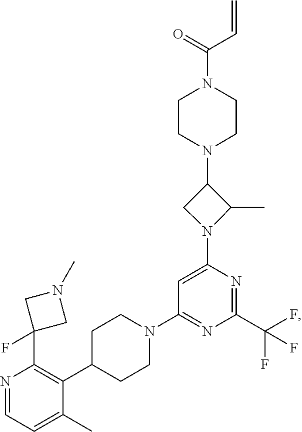 [SMILES: C=CC(=O)N1CCN(C2CN(c3cc(N4CCC(c5c(C)ccnc5C5(F)CN(C)C5)CC4)nc(C(F)(F)F)n3)C2C)CC1]

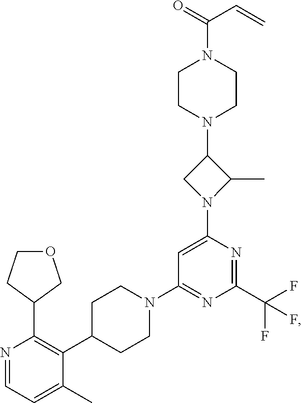 [SMILES: C=CC(=O)N1CCN(C2CN(c3cc(N4CCC(c5c(C)ccnc5C5CCOC5)CC4)nc(C(F)(F)F)n3)C2C)CC1]

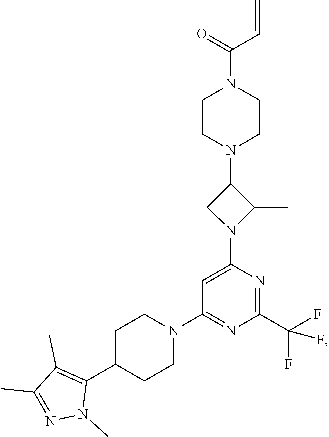 [SMILES: C=CC(=O)N1CCN(C2CN(c3cc(N4CCC(c5c(C)c(C)nn5C)CC4)nc(C(F)(F)F)n3)C2C)CC1]

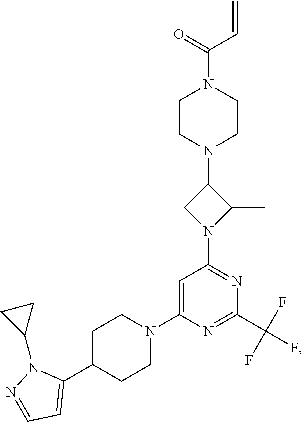 [SMILES: C=CC(=O)N1CCN(C2CN(c3cc(N4CCC(c5ccnn5C5CC5)CC4)nc(C(F)(F)F)n3)C2C)CC1]

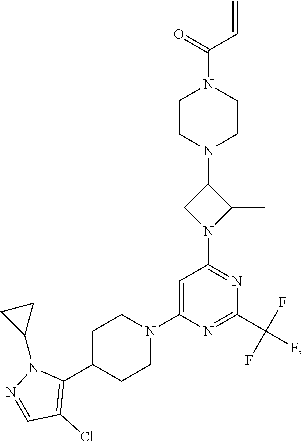 [SMILES: C=CC(=O)N1CCN(C2CN(c3cc(N4CCC(c5c(Cl)cnn5C5CC5)CC4)nc(C(F)(F)F)n3)C2C)CC1]

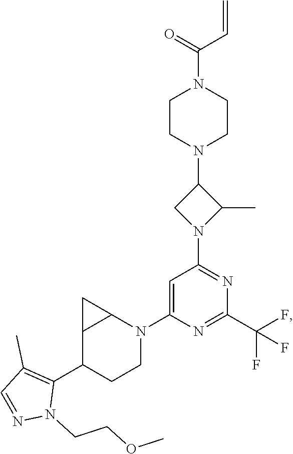 [SMILES: C=CC(=O)N1CCN(C2CN(c3cc(N4CCC(c5c(C)cnn5CCOC)C5CC54)nc(C(F)(F)F)n3)C2C)CC1]

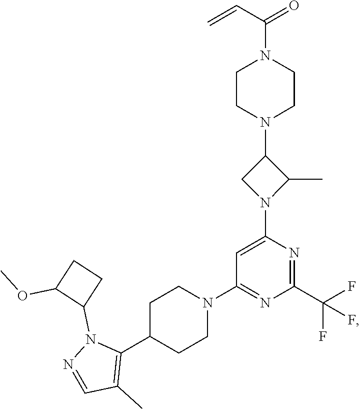 [SMILES: C=CC(=O)N1CCN(C2CN(c3cc(N4CCC(c5c(C)cnn5C5CCC5OC)CC4)nc(C(F)(F)F)n3)C2C)CC1]

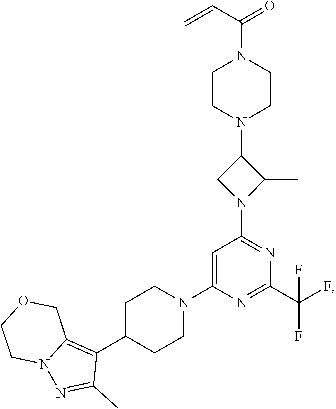 [SMILES: C=CC(=O)N1CCN(C2CN(c3cc(N4CCC(c5c(C)nn6c5COCC6)CC4)nc(C(F)(F)F)n3)C2C)CC1]

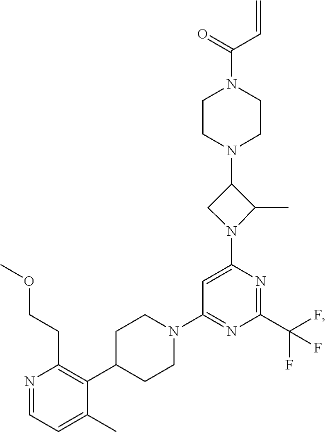 [SMILES: C=CC(=O)N1CCN(C2CN(c3cc(N4CCC(c5c(C)ccnc5CCOC)CC4)nc(C(F)(F)F)n3)C2C)CC1]

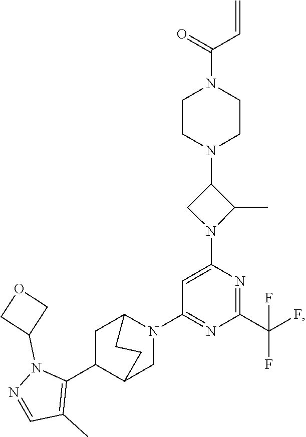 [SMILES: C=CC(=O)N1CCN(C2CN(c3cc(N4CC5CCC4CC5c4c(C)cnn4C4COC4)nc(C(F)(F)F)n3)C2C)CC1]

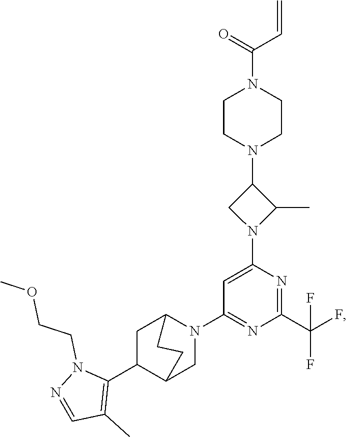 [SMILES: C=CC(=O)N1CCN(C2CN(c3cc(N4CC5CCC4CC5c4c(C)cnn4CCOC)nc(C(F)(F)F)n3)C2C)CC1]

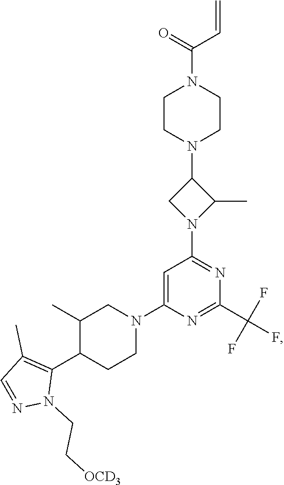

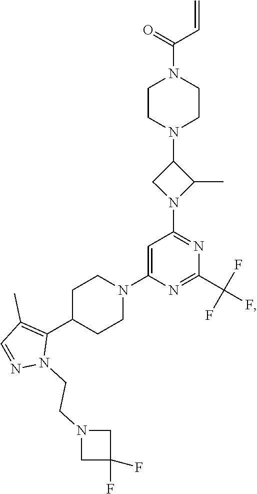 [SMILES: C=CC(=O)N1CCN(C2CN(c3cc(N4CCC(c5c(C)cnn5CCN5CC(F)(F)C5)CC4)nc(C(F)(F)F)n3)C2C)CC1]

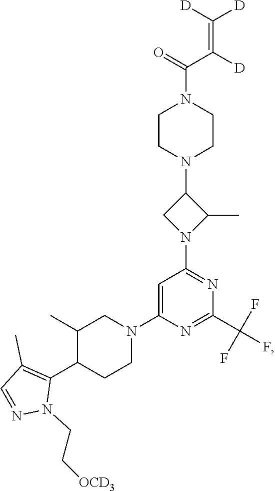

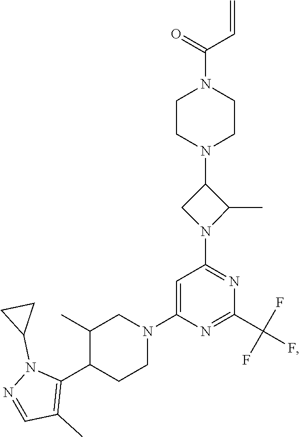 [SMILES: C=CC(=O)N1CCN(C2CN(c3cc(N4CCC(c5c(C)cnn5C5CC5)C(C)C4)nc(C(F)(F)F)n3)C2C)CC1]

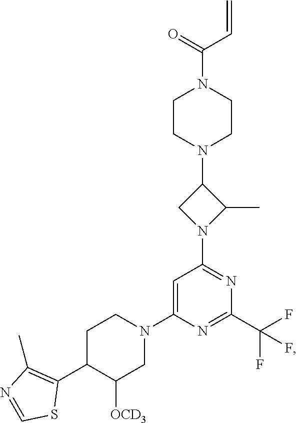

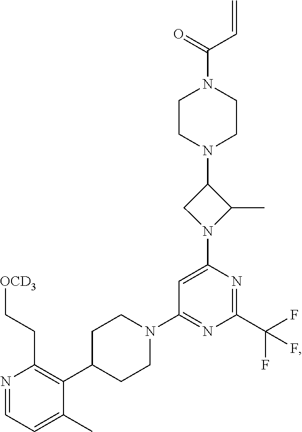

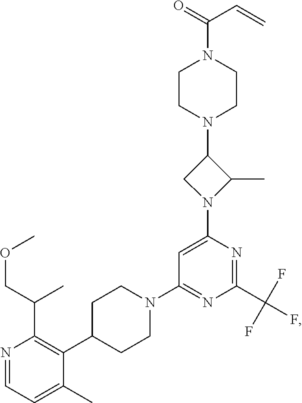 [SMILES: C=CC(=O)N1CCN(C2CN(c3cc(N4CCC(c5c(C)ccnc5C(C)COC)CC4)nc(C(F)(F)F)n3)C2C)CC1]

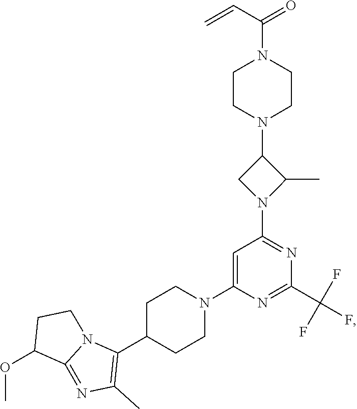 [SMILES: C=CC(=O)N1CCN(C2CN(c3cc(N4CCC(c5c(C)nc6n5CCC6OC)CC4)nc(C(F)(F)F)n3)C2C)CC1]

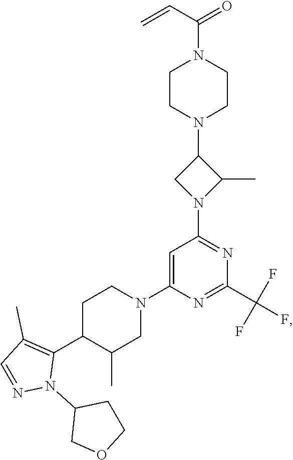 [SMILES: C=CC(=O)N1CCN(C2CN(c3cc(N4CCC(c5c(C)cnn5C5CCOC5)C(C)C4)nc(C(F)(F)F)n3)C2C)CC1]

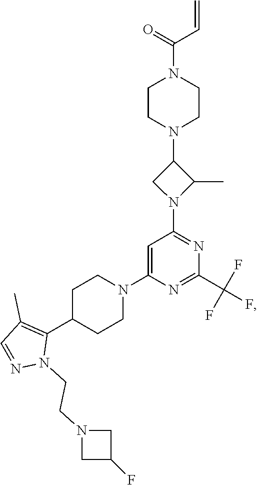 [SMILES: C=CC(=O)N1CCN(C2CN(c3cc(N4CCC(c5c(C)cnn5CCN5CC(F)C5)CC4)nc(C(F)(F)F)n3)C2C)CC1]

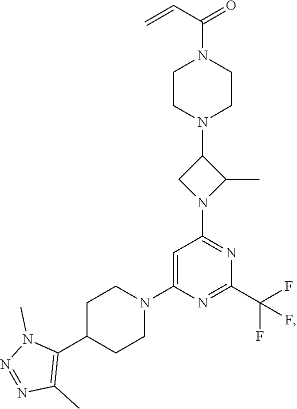 [SMILES: C=CC(=O)N1CCN(C2CN(c3cc(N4CCC(c5c(C)nnn5C)CC4)nc(C(F)(F)F)n3)C2C)CC1]

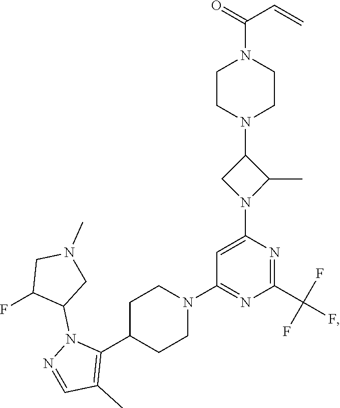 [SMILES: C=CC(=O)N1CCN(C2CN(c3cc(N4CCC(c5c(C)cnn5C5CN(C)CC5F)CC4)nc(C(F)(F)F)n3)C2C)CC1]

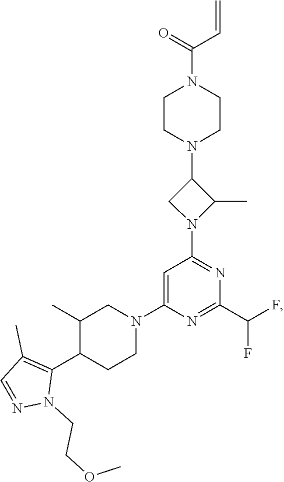 [SMILES: C=CC(=O)N1CCN(C2CN(c3cc(N4CCC(c5c(C)cnn5CCOC)C(C)C4)nc(C(F)F)n3)C2C)CC1]

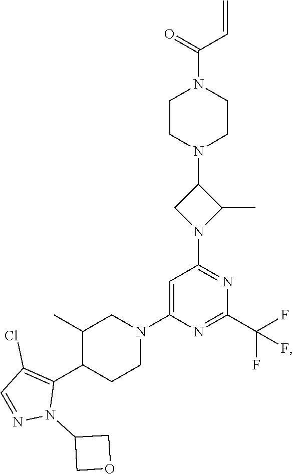 [SMILES: C=CC(=O)N1CCN(C2CN(c3cc(N4CCC(c5c(Cl)cnn5C5COC5)C(C)C4)nc(C(F)(F)F)n3)C2C)CC1]

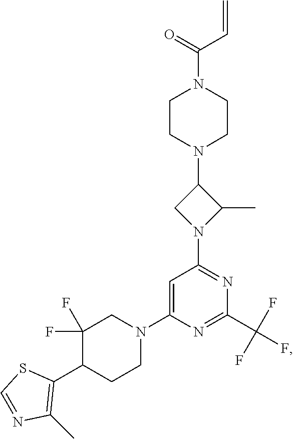 [SMILES: C=CC(=O)N1CCN(C2CN(c3cc(N4CCC(c5scnc5C)C(F)(F)C4)nc(C(F)(F)F)n3)C2C)CC1]

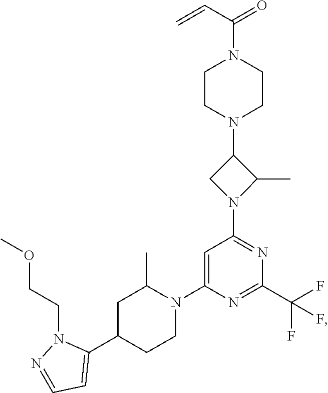 [SMILES: C=CC(=O)N1CCN(C2CN(c3cc(N4CCC(c5ccnn5CCOC)CC4C)nc(C(F)(F)F)n3)C2C)CC1]

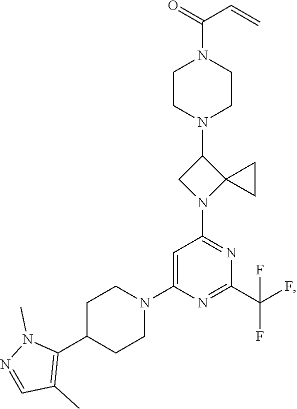 [SMILES: C=CC(=O)N1CCN(C2CN(c3cc(N4CCC(c5c(C)cnn5C)CC4)nc(C(F)(F)F)n3)C23CC3)CC1]

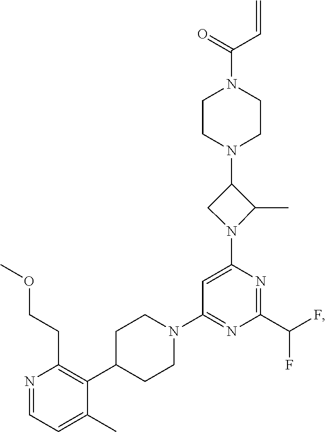 [SMILES: C=CC(=O)N1CCN(C2CN(c3cc(N4CCC(c5c(C)ccnc5CCOC)CC4)nc(C(F)F)n3)C2C)CC1]

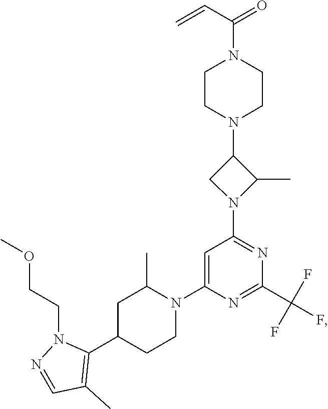 [SMILES: C=CC(=O)N1CCN(C2CN(c3cc(N4CCC(c5c(C)cnn5CCOC)CC4C)nc(C(F)(F)F)n3)C2C)CC1]

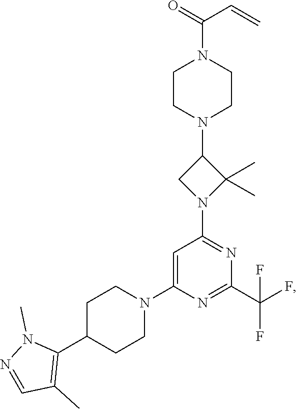 [SMILES: C=CC(=O)N1CCN(C2CN(c3cc(N4CCC(c5c(C)cnn5C)CC4)nc(C(F)(F)F)n3)C2(C)C)CC1]

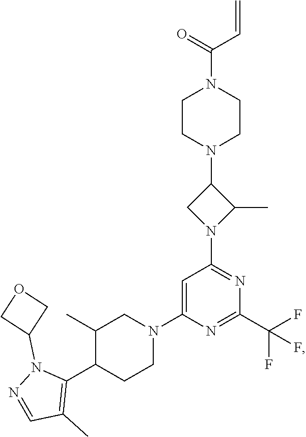 [SMILES: C=CC(=O)N1CCN(C2CN(c3cc(N4CCC(c5c(C)cnn5C5COC5)C(C)C4)nc(C(F)(F)F)n3)C2C)CC1]

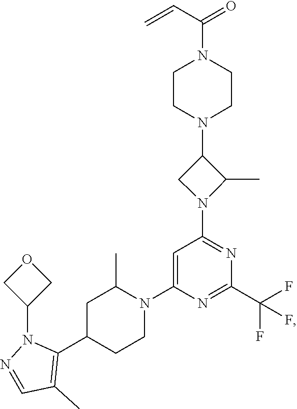 [SMILES: C=CC(=O)N1CCN(C2CN(c3cc(N4CCC(c5c(C)cnn5C5COC5)CC4C)nc(C(F)(F)F)n3)C2C)CC1]

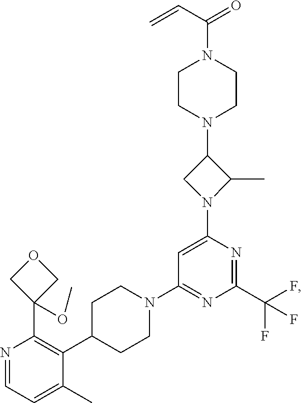 [SMILES: C=CC(=O)N1CCN(C2CN(c3cc(N4CCC(c5c(C)ccnc5C5(OC)COC5)CC4)nc(C(F)(F)F)n3)C2C)CC1]

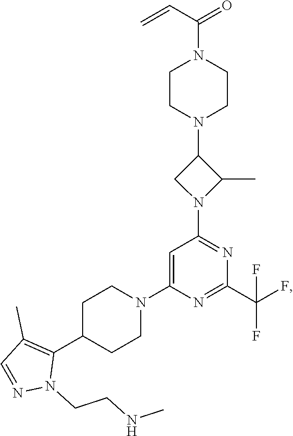 [SMILES: C=CC(=O)N1CCN(C2CN(c3cc(N4CCC(c5c(C)cnn5CCNC)CC4)nc(C(F)(F)F)n3)C2C)CC1]

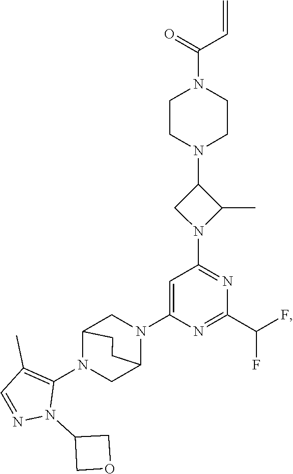 [SMILES: C=CC(=O)N1CCN(C2CN(c3cc(N4CC5CCC4CN5c4c(C)cnn4C4COC4)nc(C(F)F)n3)C2C)CC1]

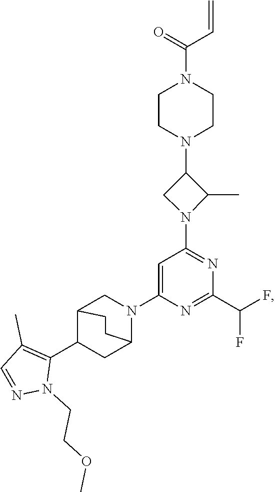 [SMILES: C=CC(=O)N1CCN(C2CN(c3cc(N4CC5CCC4CC5c4c(C)cnn4CCOC)nc(C(F)F)n3)C2C)CC1]

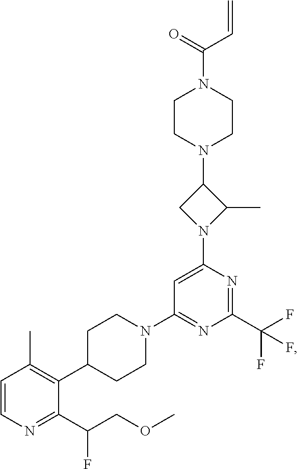 [SMILES: C=CC(=O)N1CCN(C2CN(c3cc(N4CCC(c5c(C)ccnc5C(F)COC)CC4)nc(C(F)(F)F)n3)C2C)CC1]

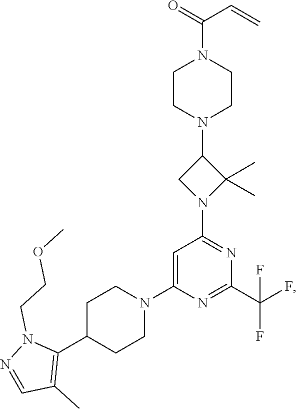 [SMILES: C=CC(=O)N1CCN(C2CN(c3cc(N4CCC(c5c(C)cnn5CCOC)CC4)nc(C(F)(F)F)n3)C2(C)C)CC1]

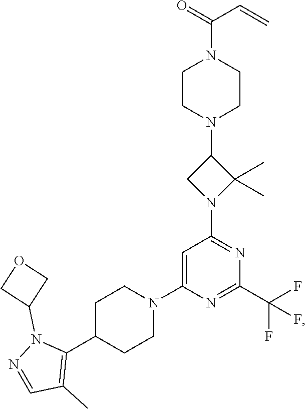 [SMILES: C=CC(=O)N1CCN(C2CN(c3cc(N4CCC(c5c(C)cnn5C5COC5)CC4)nc(C(F)(F)F)n3)C2(C)C)CC1]

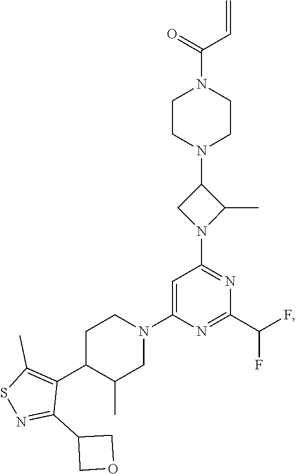 [SMILES: C=CC(=O)N1CCN(C2CN(c3cc(N4CCC(c5c(C6COC6)nsc5C)C(C)C4)nc(C(F)F)n3)C2C)CC1]

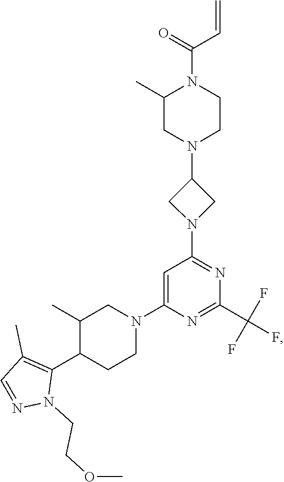 [SMILES: C=CC(=O)N1CCN(C2CN(c3cc(N4CCC(c5c(C)cnn5CCOC)C(C)C4)nc(C(F)(F)F)n3)C2)CC1C]

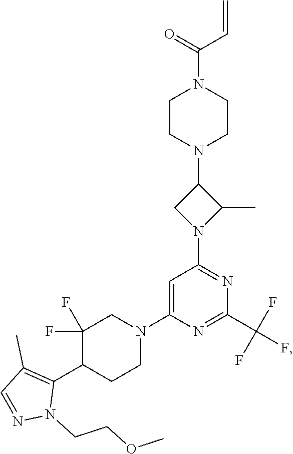 [SMILES: C=CC(=O)N1CCN(C2CN(c3cc(N4CCC(c5c(C)cnn5CCOC)C(F)(F)C4)nc(C(F)(F)F)n3)C2C)CC1]

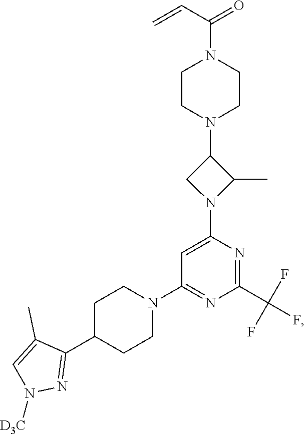 [SMILES: [2H]C([2H])([2H])n1cc(C)c(C2CCN(c3cc(N4CC(N5CCN(C(=O)C=C)CC5)C4C)nc(C(F)(F)F)n3)CC2)n1]

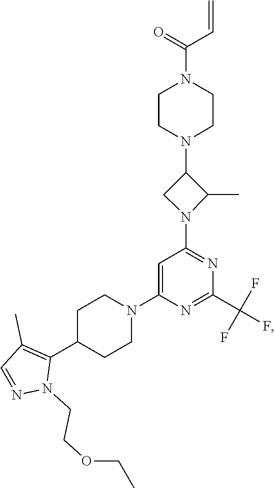 [SMILES: C=CC(=O)N1CCN(C2CN(c3cc(N4CCC(c5c(C)cnn5CCOCC)CC4)nc(C(F)(F)F)n3)C2C)CC1]

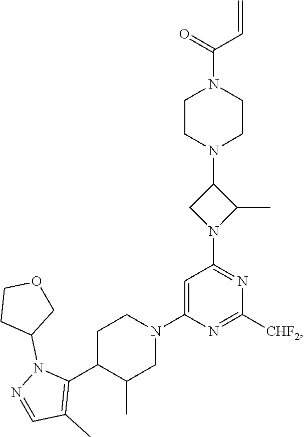 [SMILES: C=CC(=O)N1CCN(C2CN(c3cc(N4CCC(c5c(C)cnn5C5CCOC5)C(C)C4)nc(C(F)F)n3)C2C)CC1]

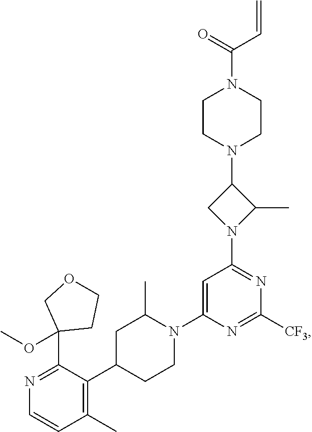 [SMILES: C=CC(=O)N1CCN(C2CN(c3cc(N4CCC(c5c(C)ccnc5C5(OC)CCOC5)CC4C)nc(C(F)(F)F)n3)C2C)CC1]

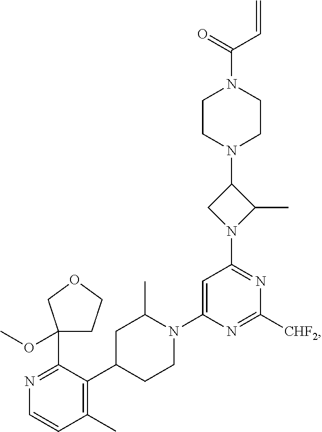 [SMILES: C=CC(=O)N1CCN(C2CN(c3cc(N4CCC(c5c(C)ccnc5C5(OC)CCOC5)CC4C)nc(C(F)F)n3)C2C)CC1]

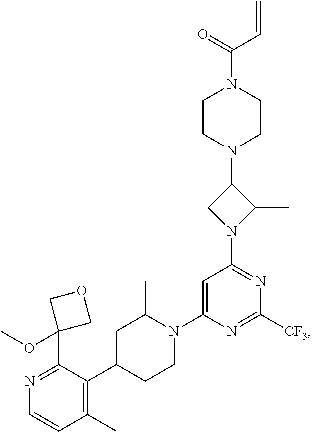 [SMILES: C=CC(=O)N1CCN(C2CN(c3cc(N4CCC(c5c(C)ccnc5C5(OC)COC5)CC4C)nc(C(F)(F)F)n3)C2C)CC1]

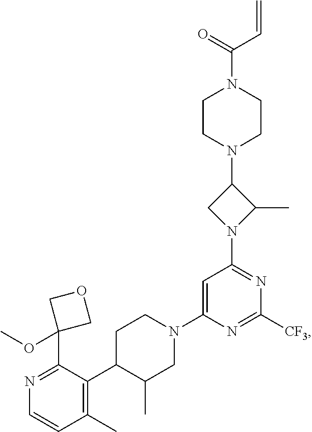 [SMILES: C=CC(=O)N1CCN(C2CN(c3cc(N4CCC(c5c(C)ccnc5C5(OC)COC5)C(C)C4)nc(C(F)(F)F)n3)C2C)CC1]

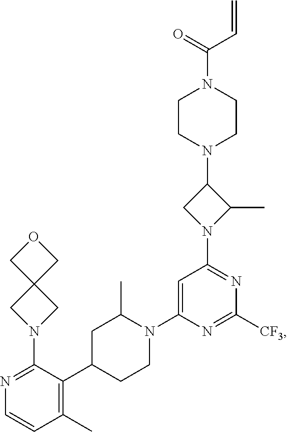 [SMILES: C=CC(=O)N1CCN(C2CN(c3cc(N4CCC(c5c(C)ccnc5N5CC6(COC6)C5)CC4C)nc(C(F)(F)F)n3)C2C)CC1]

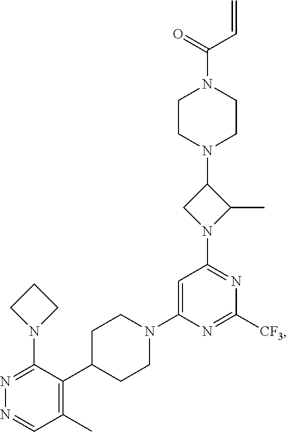 [SMILES: C=CC(=O)N1CCN(C2CN(c3cc(N4CCC(c5c(C)cnnc5N5CCC5)CC4)nc(C(F)(F)F)n3)C2C)CC1]

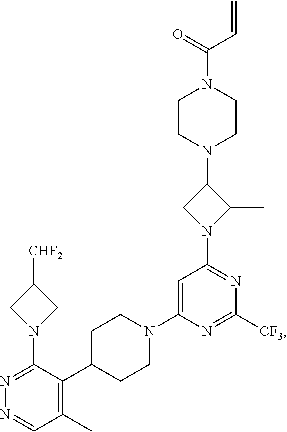 [SMILES: C=CC(=O)N1CCN(C2CN(c3cc(N4CCC(c5c(C)cnnc5N5CC(C(F)F)C5)CC4)nc(C(F)(F)F)n3)C2C)CC1]

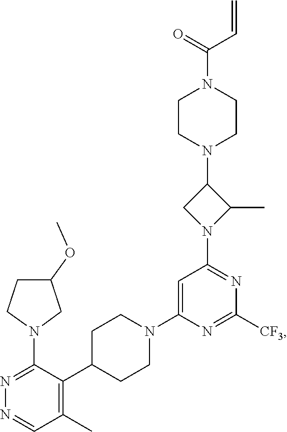 [SMILES: C=CC(=O)N1CCN(C2CN(c3cc(N4CCC(c5c(C)cnnc5N5CCC(OC)C5)CC4)nc(C(F)(F)F)n3)C2C)CC1]

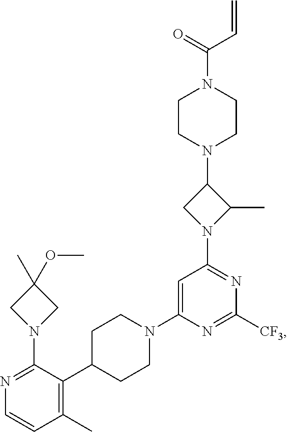 [SMILES: C=CC(=O)N1CCN(C2CN(c3cc(N4CCC(c5c(C)ccnc5N5CC(C)(OC)C5)CC4)nc(C(F)(F)F)n3)C2C)CC1]

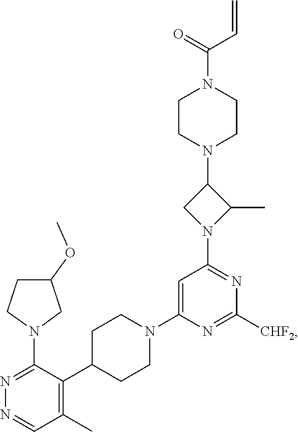 [SMILES: C=CC(=O)N1CCN(C2CN(c3cc(N4CCC(c5c(C)cnnc5N5CCC(OC)C5)CC4)nc(C(F)F)n3)C2C)CC1]

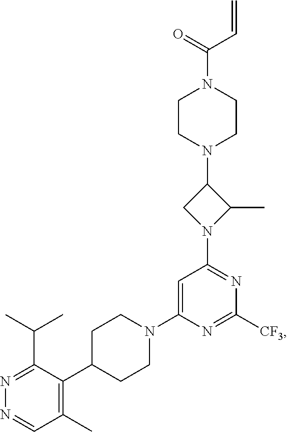 [SMILES: C=CC(=O)N1CCN(C2CN(c3cc(N4CCC(c5c(C)cnnc5C(C)C)CC4)nc(C(F)(F)F)n3)C2C)CC1]

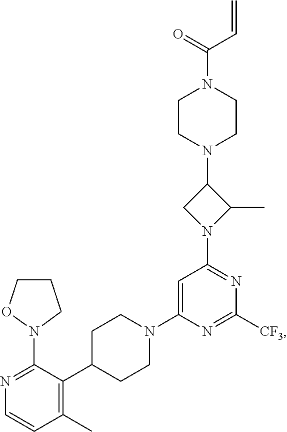 [SMILES: C=CC(=O)N1CCN(C2CN(c3cc(N4CCC(c5c(C)ccnc5N5CCCO5)CC4)nc(C(F)(F)F)n3)C2C)CC1]

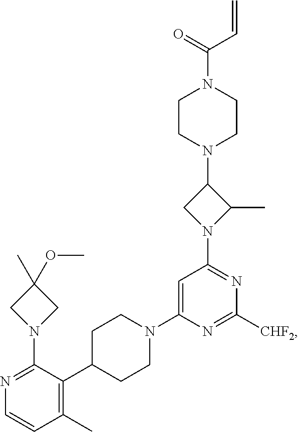 [SMILES: C=CC(=O)N1CCN(C2CN(c3cc(N4CCC(c5c(C)ccnc5N5CC(C)(OC)C5)CC4)nc(C(F)F)n3)C2C)CC1]

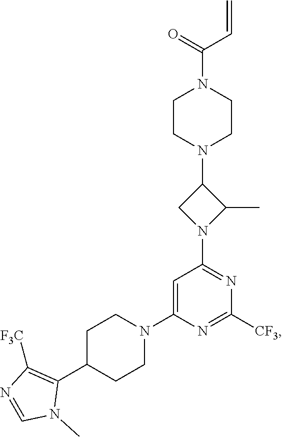 [SMILES: C=CC(=O)N1CCN(C2CN(c3cc(N4CCC(c5c(C(F)(F)F)ncn5C)CC4)nc(C(F)(F)F)n3)C2C)CC1]

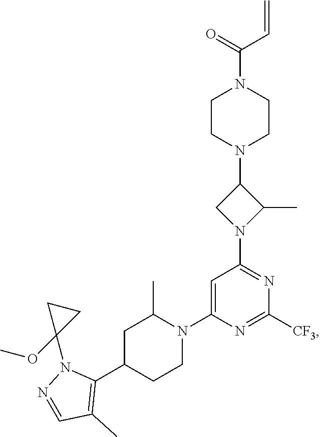 [SMILES: C=CC(=O)N1CCN(C2CN(c3cc(N4CCC(c5c(C)cnn5C5(OC)CC5)CC4C)nc(C(F)(F)F)n3)C2C)CC1]

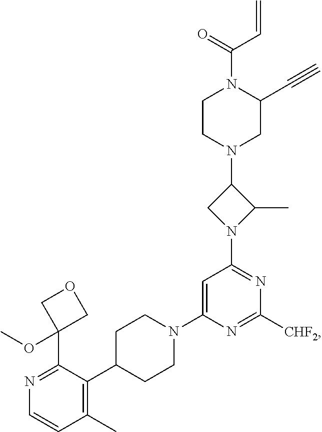 [SMILES: C#CC1CN(C2CN(c3cc(N4CCC(c5c(C)ccnc5C5(OC)COC5)CC4)nc(C(F)F)n3)C2C)CCN1C(=O)C=C]

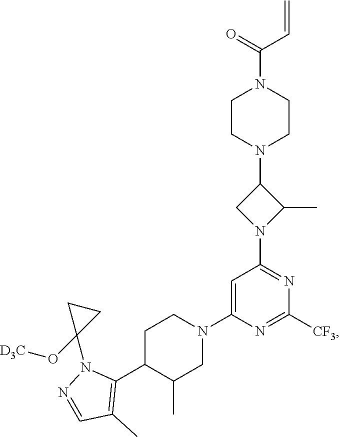 [SMILES: [2H]C([2H])([2H])OC1(n2ncc(C)c2C2CCN(c3cc(N4CC(N5CCN(C(=O)C=C)CC5)C4C)nc(C(F)(F)F)n3)CC2C)CC1]

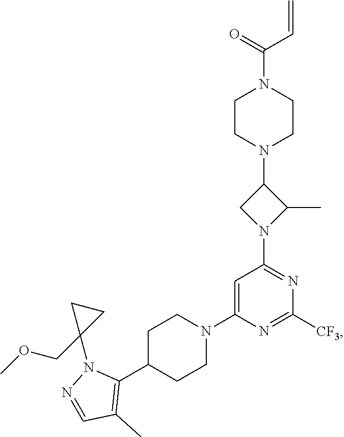 [SMILES: C=CC(=O)N1CCN(C2CN(c3cc(N4CCC(c5c(C)cnn5C5(COC)CC5)CC4)nc(C(F)(F)F)n3)C2C)CC1]

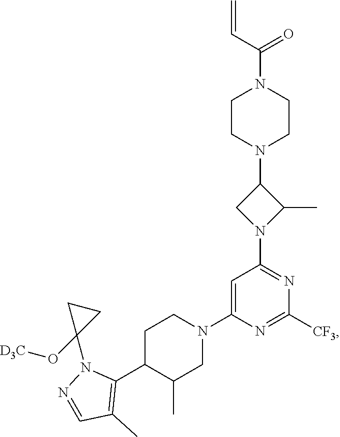 [SMILES: [2H]C([2H])([2H])OC1(n2ncc(C)c2C2CCN(c3cc(N4CC(N5CCN(C(=O)C=C)CC5)C4C)nc(C(F)(F)F)n3)CC2C)CC1]

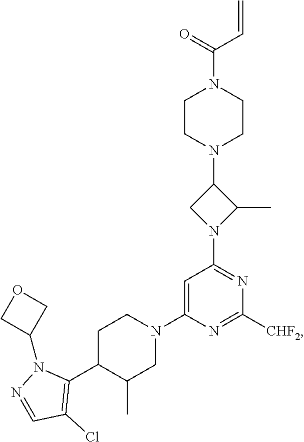 [SMILES: C=CC(=O)N1CCN(C2CN(c3cc(N4CCC(c5c(Cl)cnn5C5COC5)C(C)C4)nc(C(F)F)n3)C2C)CC1]

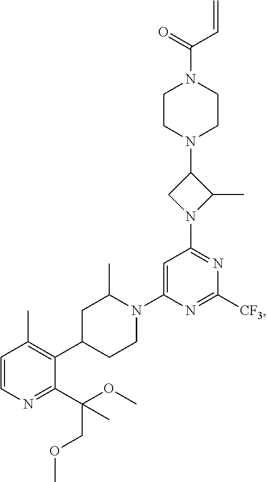 [SMILES: C=CC(=O)N1CCN(C2CN(c3cc(N4CCC(c5c(C)ccnc5C(C)(COC)OC)CC4C)nc(C(F)(F)F)n3)C2C)CC1]

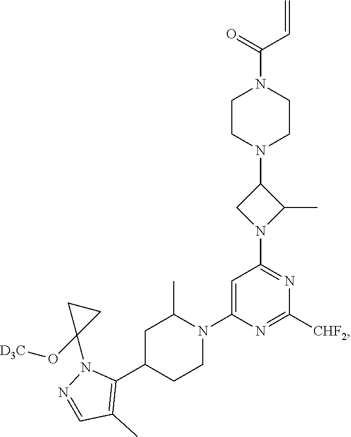 [SMILES: [2H]C([2H])([2H])OC1(n2ncc(C)c2C2CCN(c3cc(N4CC(N5CCN(C(=O)C=C)CC5)C4C)nc(C(F)F)n3)C(C)C2)CC1]

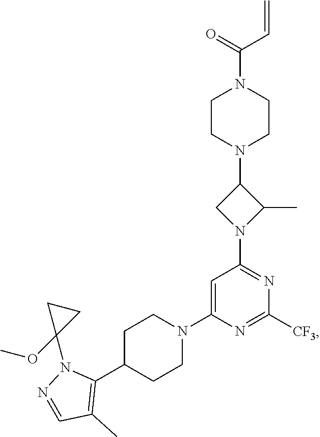 [SMILES: C=CC(=O)N1CCN(C2CN(c3cc(N4CCC(c5c(C)cnn5C5(OC)CC5)CC4)nc(C(F)(F)F)n3)C2C)CC1]

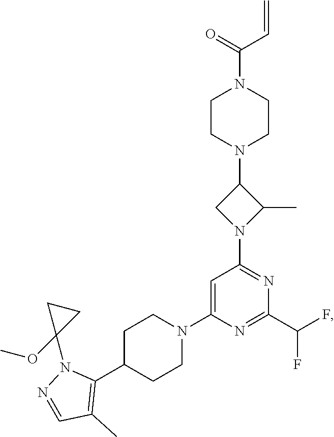 [SMILES: C=CC(=O)N1CCN(C2CN(c3cc(N4CCC(c5c(C)cnn5C5(OC)CC5)CC4)nc(C(F)F)n3)C2C)CC1]

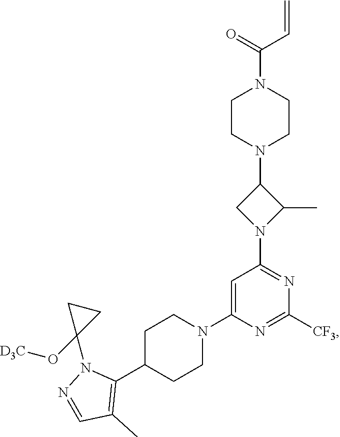 [SMILES: [2H]C([2H])([2H])OC1(n2ncc(C)c2C2CCN(c3cc(N4CC(N5CCN(C(=O)C=C)CC5)C4C)nc(C(F)(F)F)n3)CC2)CC1]

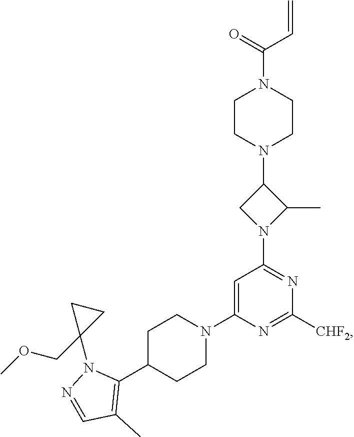 [SMILES: C=CC(=O)N1CCN(C2CN(c3cc(N4CCC(c5c(C)cnn5C5(COC)CC5)CC4)nc(C(F)F)n3)C2C)CC1]

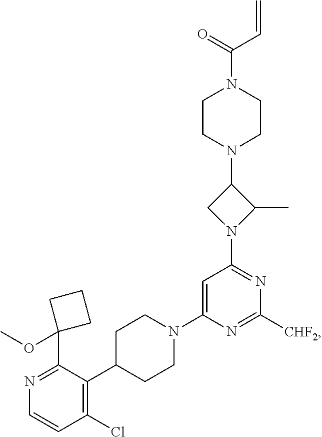 [SMILES: C=CC(=O)N1CCN(C2CN(c3cc(N4CCC(c5c(Cl)ccnc5C5(OC)CCC5)CC4)nc(C(F)F)n3)C2C)CC1]

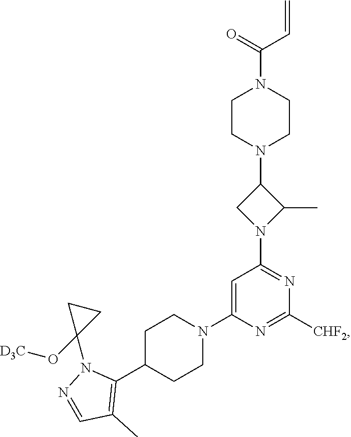 [SMILES: [2H]C([2H])([2H])OC1(n2ncc(C)c2C2CCN(c3cc(N4CC(N5CCN(C(=O)C=C)CC5)C4C)nc(C(F)F)n3)CC2)CC1]

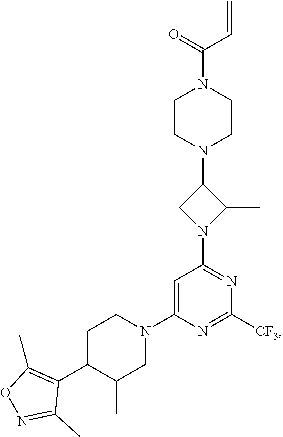 [SMILES: C=CC(=O)N1CCN(C2CN(c3cc(N4CCC(c5c(C)noc5C)C(C)C4)nc(C(F)(F)F)n3)C2C)CC1]

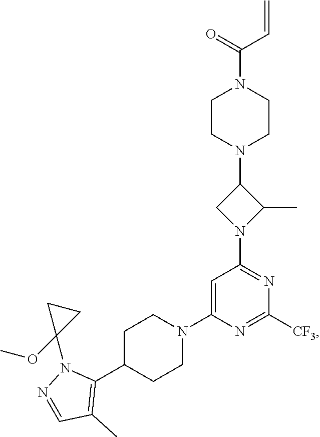 [SMILES: C=CC(=O)N1CCN(C2CN(c3cc(N4CCC(c5c(C)cnn5C5(OC)CC5)CC4)nc(C(F)(F)F)n3)C2C)CC1]

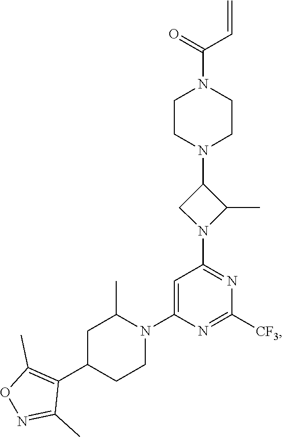 [SMILES: C=CC(=O)N1CCN(C2CN(c3cc(N4CCC(c5c(C)noc5C)CC4C)nc(C(F)(F)F)n3)C2C)CC1]

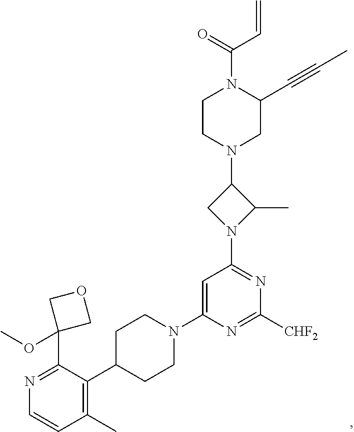 [SMILES: C=CC(=O)N1CCN(C2CN(c3cc(N4CCC(c5c(C)ccnc5C5(OC)COC5)CC4)nc(C(F)F)n3)C2C)CC1C#CC]

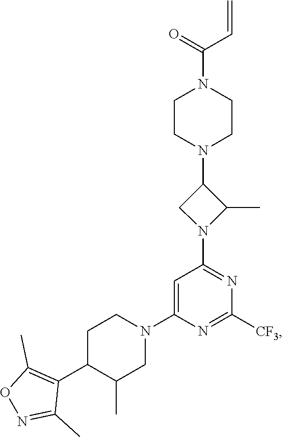 [SMILES: C=CC(=O)N1CCN(C2CN(c3cc(N4CCC(c5c(C)noc5C)C(C)C4)nc(C(F)(F)F)n3)C2C)CC1]

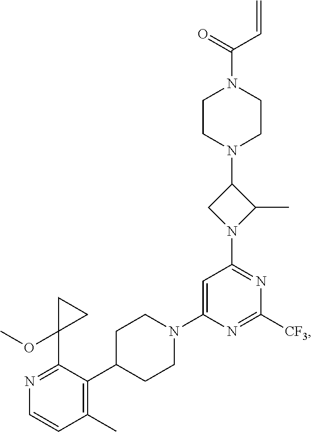 [SMILES: C=CC(=O)N1CCN(C2CN(c3cc(N4CCC(c5c(C)ccnc5C5(OC)CC5)CC4)nc(C(F)(F)F)n3)C2C)CC1]

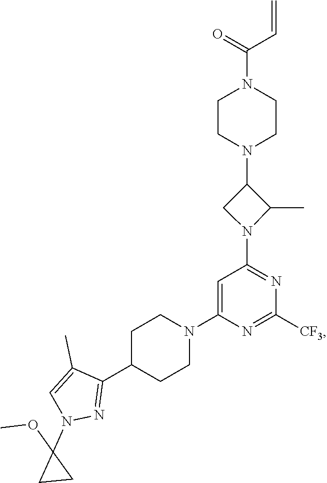 [SMILES: C=CC(=O)N1CCN(C2CN(c3cc(N4CCC(c5nn(C6(OC)CC6)cc5C)CC4)nc(C(F)(F)F)n3)C2C)CC1]

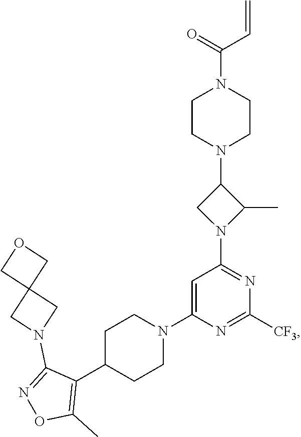 [SMILES: C=CC(=O)N1CCN(C2CN(c3cc(N4CCC(c5c(N6CC7(COC7)C6)noc5C)CC4)nc(C(F)(F)F)n3)C2C)CC1]

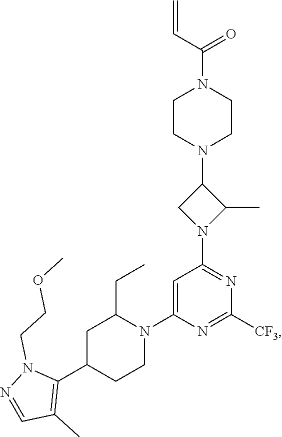 [SMILES: C=CC(=O)N1CCN(C2CN(c3cc(N4CCC(c5c(C)cnn5CCOC)CC4CC)nc(C(F)(F)F)n3)C2C)CC1]

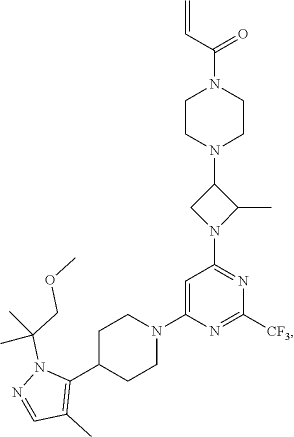 [SMILES: C=CC(=O)N1CCN(C2CN(c3cc(N4CCC(c5c(C)cnn5C(C)(C)COC)CC4)nc(C(F)(F)F)n3)C2C)CC1]

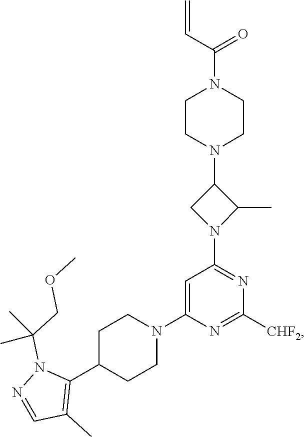 [SMILES: C=CC(=O)N1CCN(C2CN(c3cc(N4CCC(c5c(C)cnn5C(C)(C)COC)CC4)nc(C(F)F)n3)C2C)CC1]

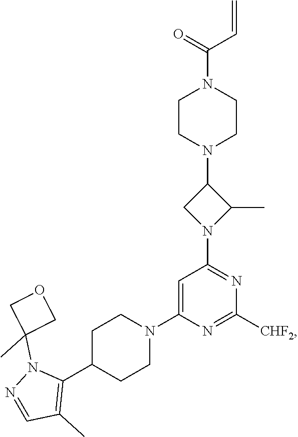 [SMILES: C=CC(=O)N1CCN(C2CN(c3cc(N4CCC(c5c(C)cnn5C5(C)COC5)CC4)nc(C(F)F)n3)C2C)CC1]

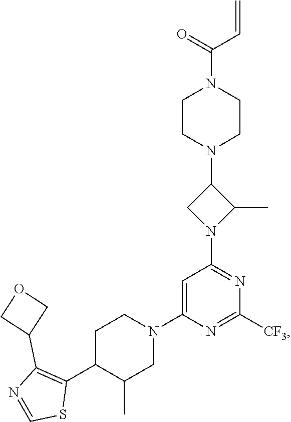 [SMILES: C=CC(=O)N1CCN(C2CN(c3cc(N4CCC(c5scnc5C5COC5)C(C)C4)nc(C(F)(F)F)n3)C2C)CC1]

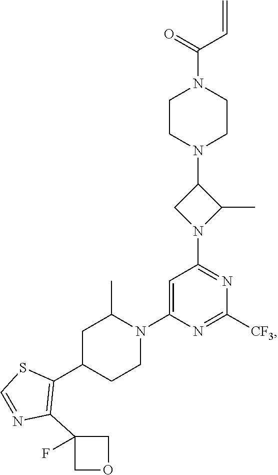 [SMILES: C=CC(=O)N1CCN(C2CN(c3cc(N4CCC(c5scnc5C5(F)COC5)CC4C)nc(C(F)(F)F)n3)C2C)CC1]

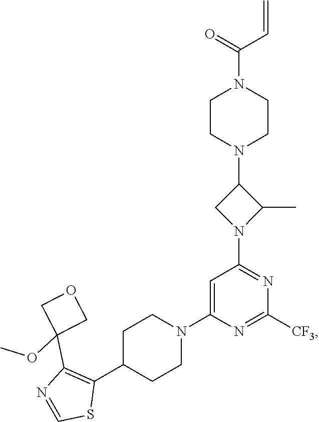 [SMILES: C=CC(=O)N1CCN(C2CN(c3cc(N4CCC(c5scnc5C5(OC)COC5)CC4)nc(C(F)(F)F)n3)C2C)CC1]

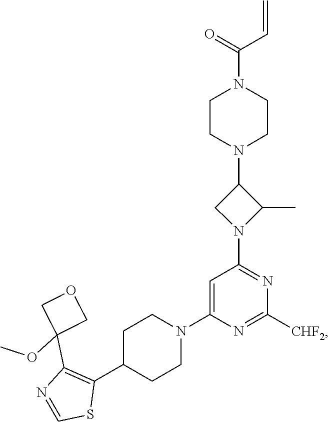 [SMILES: C=CC(=O)N1CCN(C2CN(c3cc(N4CCC(c5scnc5C5(OC)COC5)CC4)nc(C(F)F)n3)C2C)CC1]

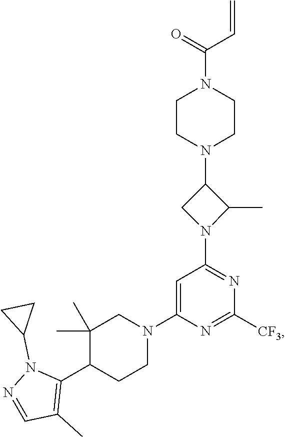 [SMILES: C=CC(=O)N1CCN(C2CN(c3cc(N4CCC(c5c(C)cnn5C5CC5)C(C)(C)C4)nc(C(F)(F)F)n3)C2C)CC1]

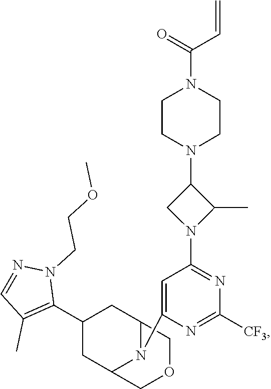 [SMILES: C=CC(=O)N1CCN(C2CN(c3cc(N4C5COCC4CC(c4c(C)cnn4CCOC)C5)nc(C(F)(F)F)n3)C2C)CC1]

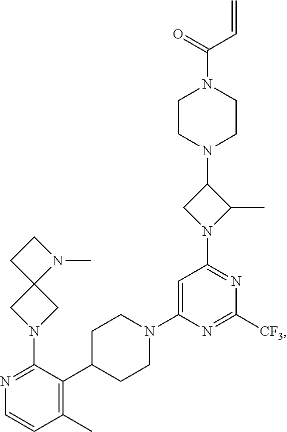 [SMILES: C=CC(=O)N1CCN(C2CN(c3cc(N4CCC(c5c(C)ccnc5N5CC6(CCN6C)C5)CC4)nc(C(F)(F)F)n3)C2C)CC1]

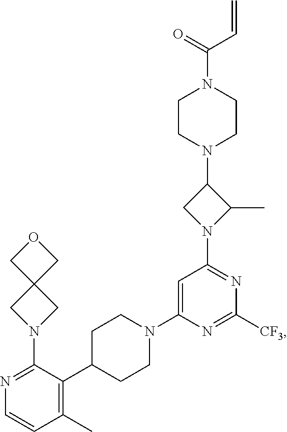 [SMILES: C=CC(=O)N1CCN(C2CN(c3cc(N4CCC(c5c(C)ccnc5N5CC6(COC6)C5)CC4)nc(C(F)(F)F)n3)C2C)CC1]

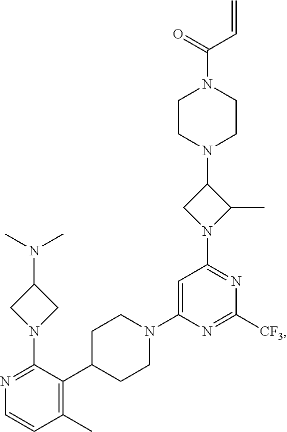 [SMILES: C=CC(=O)N1CCN(C2CN(c3cc(N4CCC(c5c(C)ccnc5N5CC(N(C)C)C5)CC4)nc(C(F)(F)F)n3)C2C)CC1]

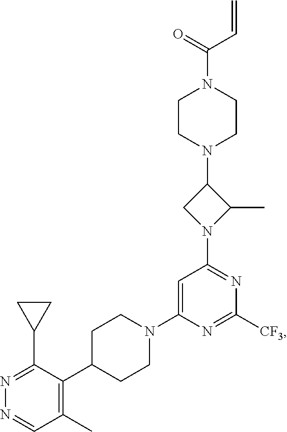 [SMILES: C=CC(=O)N1CCN(C2CN(c3cc(N4CCC(c5c(C)cnnc5C5CC5)CC4)nc(C(F)(F)F)n3)C2C)CC1]

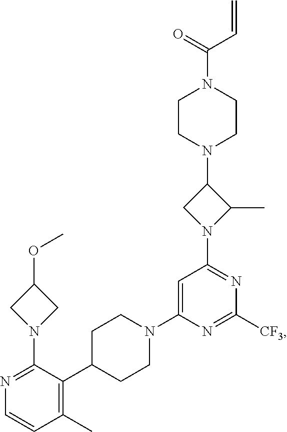 [SMILES: C=CC(=O)N1CCN(C2CN(c3cc(N4CCC(c5c(C)ccnc5N5CC(OC)C5)CC4)nc(C(F)(F)F)n3)C2C)CC1]

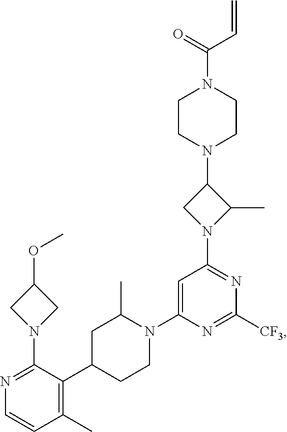 [SMILES: C=CC(=O)N1CCN(C2CN(c3cc(N4CCC(c5c(C)ccnc5N5CC(OC)C5)CC4C)nc(C(F)(F)F)n3)C2C)CC1]

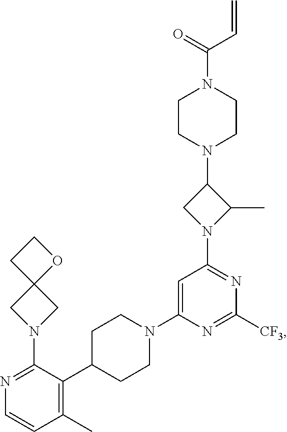 [SMILES: C=CC(=O)N1CCN(C2CN(c3cc(N4CCC(c5c(C)ccnc5N5CC6(CCO6)C5)CC4)nc(C(F)(F)F)n3)C2C)CC1]

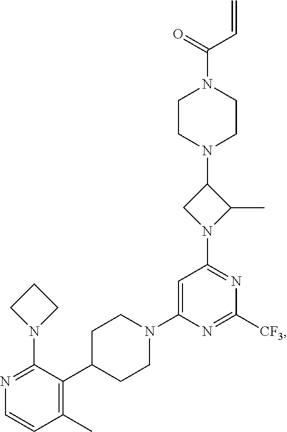 [SMILES: C=CC(=O)N1CCN(C2CN(c3cc(N4CCC(c5c(C)ccnc5N5CCC5)CC4)nc(C(F)(F)F)n3)C2C)CC1]

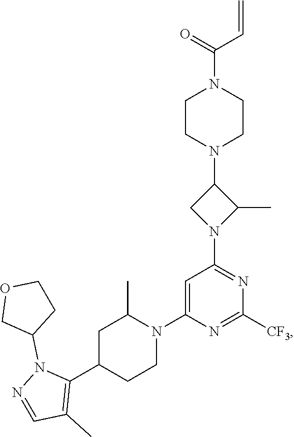 [SMILES: C=CC(=O)N1CCN(C2CN(c3cc(N4CCC(c5c(C)cnn5C5CCOC5)CC4C)nc(C(F)(F)F)n3)C2C)CC1]

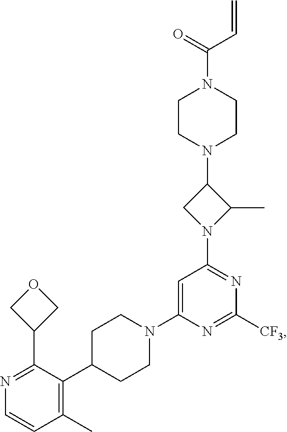 [SMILES: C=CC(=O)N1CCN(C2CN(c3cc(N4CCC(c5c(C)ccnc5C5COC5)CC4)nc(C(F)(F)F)n3)C2C)CC1]

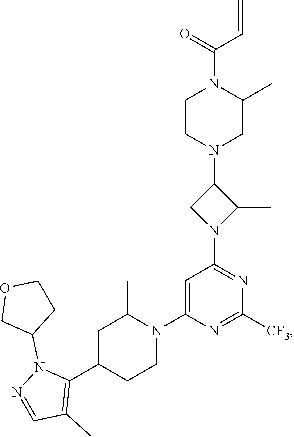 [SMILES: C=CC(=O)N1CCN(C2CN(c3cc(N4CCC(c5c(C)cnn5C5CCOC5)CC4C)nc(C(F)(F)F)n3)C2C)CC1C]

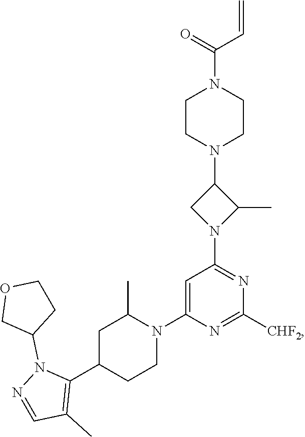 [SMILES: C=CC(=O)N1CCN(C2CN(c3cc(N4CCC(c5c(C)cnn5C5CCOC5)CC4C)nc(C(F)F)n3)C2C)CC1]

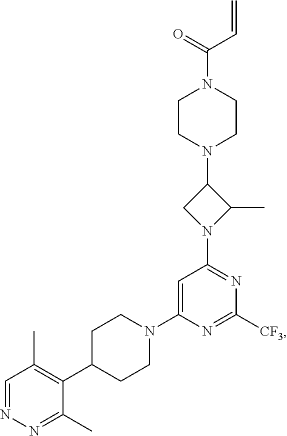 [SMILES: C=CC(=O)N1CCN(C2CN(c3cc(N4CCC(c5c(C)cnnc5C)CC4)nc(C(F)(F)F)n3)C2C)CC1]

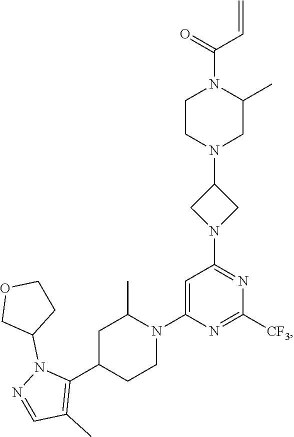 [SMILES: C=CC(=O)N1CCN(C2CN(c3cc(N4CCC(c5c(C)cnn5C5CCOC5)CC4C)nc(C(F)(F)F)n3)C2)CC1C]

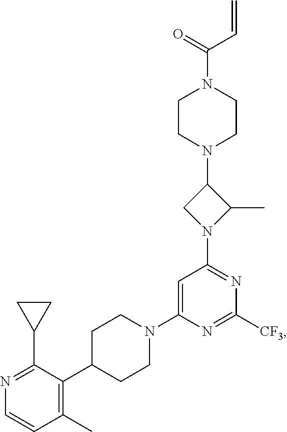 [SMILES: C=CC(=O)N1CCN(C2CN(c3cc(N4CCC(c5c(C)ccnc5C5CC5)CC4)nc(C(F)(F)F)n3)C2C)CC1]

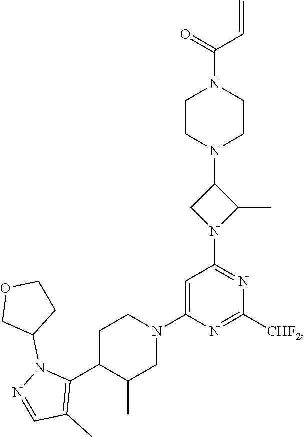 [SMILES: C=CC(=O)N1CCN(C2CN(c3cc(N4CCC(c5c(C)cnn5C5CCOC5)C(C)C4)nc(C(F)F)n3)C2C)CC1]

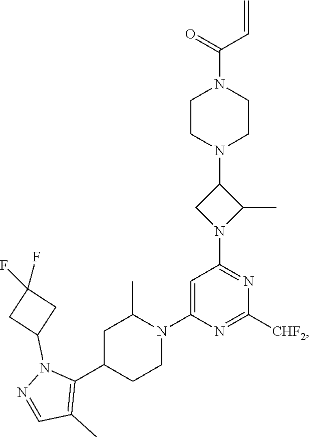 [SMILES: C=CC(=O)N1CCN(C2CN(c3cc(N4CCC(c5c(C)cnn5C5CC(F)(F)C5)CC4C)nc(C(F)F)n3)C2C)CC1]

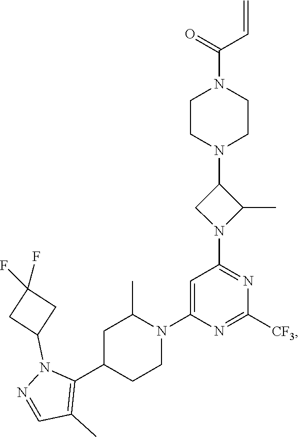 [SMILES: C=CC(=O)N1CCN(C2CN(c3cc(N4CCC(c5c(C)cnn5C5CC(F)(F)C5)CC4C)nc(C(F)(F)F)n3)C2C)CC1]

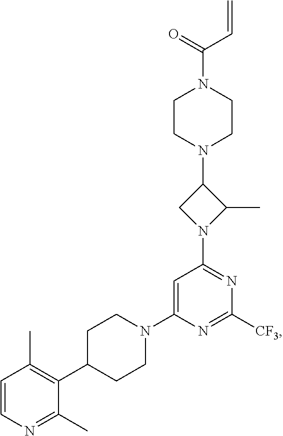 [SMILES: C=CC(=O)N1CCN(C2CN(c3cc(N4CCC(c5c(C)ccnc5C)CC4)nc(C(F)(F)F)n3)C2C)CC1]

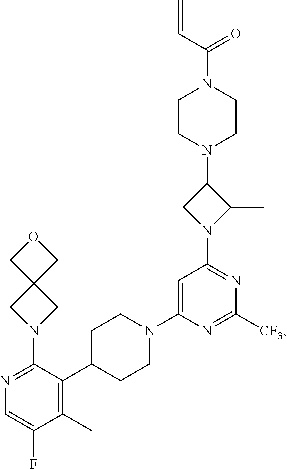 [SMILES: C=CC(=O)N1CCN(C2CN(c3cc(N4CCC(c5c(N6CC7(COC7)C6)ncc(F)c5C)CC4)nc(C(F)(F)F)n3)C2C)CC1]

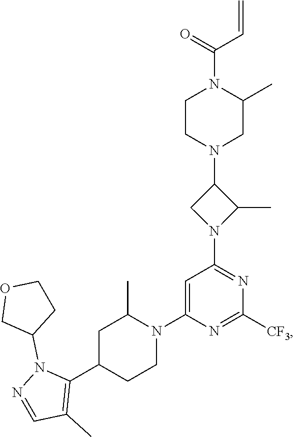 [SMILES: C=CC(=O)N1CCN(C2CN(c3cc(N4CCC(c5c(C)cnn5C5CCOC5)CC4C)nc(C(F)(F)F)n3)C2C)CC1C]

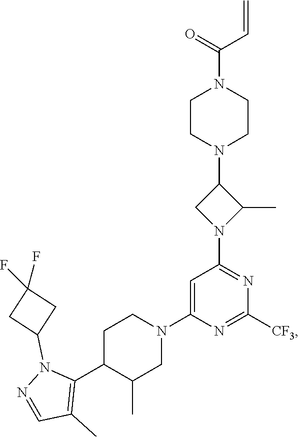 [SMILES: C=CC(=O)N1CCN(C2CN(c3cc(N4CCC(c5c(C)cnn5C5CC(F)(F)C5)C(C)C4)nc(C(F)(F)F)n3)C2C)CC1]

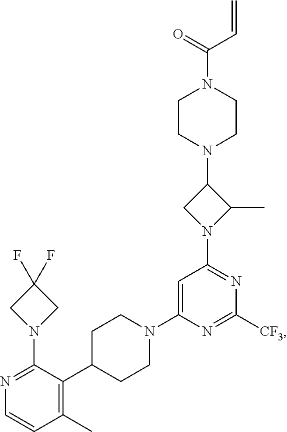 [SMILES: C=CC(=O)N1CCN(C2CN(c3cc(N4CCC(c5c(C)ccnc5N5CC(F)(F)C5)CC4)nc(C(F)(F)F)n3)C2C)CC1]

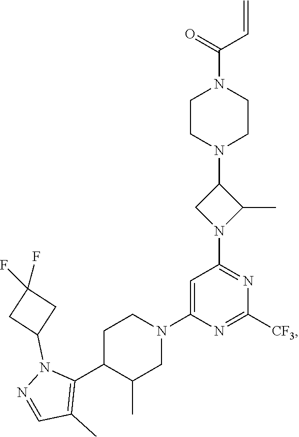 [SMILES: C=CC(=O)N1CCN(C2CN(c3cc(N4CCC(c5c(C)cnn5C5CC(F)(F)C5)C(C)C4)nc(C(F)(F)F)n3)C2C)CC1]

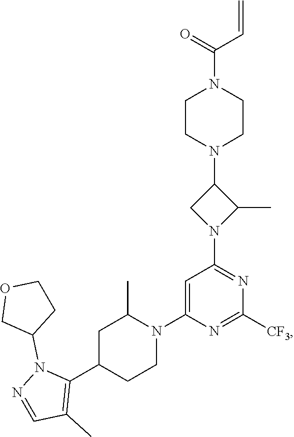 [SMILES: C=CC(=O)N1CCN(C2CN(c3cc(N4CCC(c5c(C)cnn5C5CCOC5)CC4C)nc(C(F)(F)F)n3)C2C)CC1]

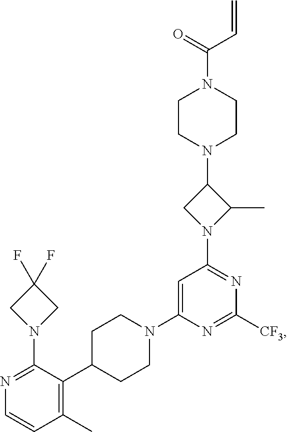 [SMILES: C=CC(=O)N1CCN(C2CN(c3cc(N4CCC(c5c(C)ccnc5N5CC(F)(F)C5)CC4)nc(C(F)(F)F)n3)C2C)CC1]

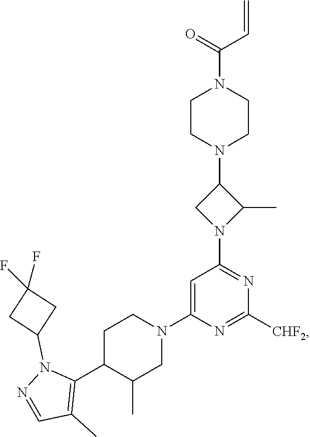 [SMILES: C=CC(=O)N1CCN(C2CN(c3cc(N4CCC(c5c(C)cnn5C5CC(F)(F)C5)C(C)C4)nc(C(F)F)n3)C2C)CC1]

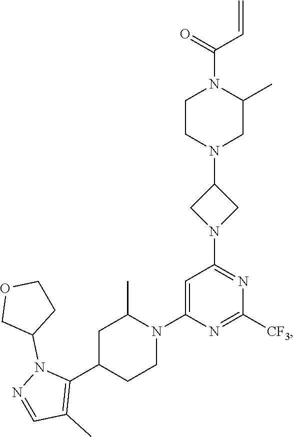 [SMILES: C=CC(=O)N1CCN(C2CN(c3cc(N4CCC(c5c(C)cnn5C5CCOC5)CC4C)nc(C(F)(F)F)n3)C2)CC1C]

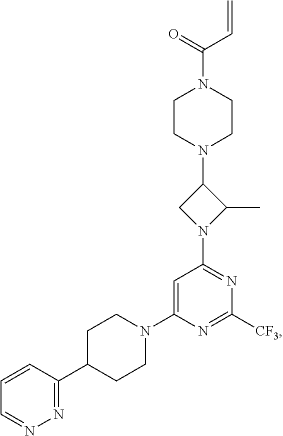 [SMILES: C=CC(=O)N1CCN(C2CN(c3cc(N4CCC(c5cccnn5)CC4)nc(C(F)(F)F)n3)C2C)CC1]

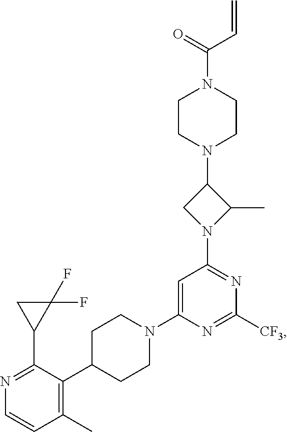 [SMILES: C=CC(=O)N1CCN(C2CN(c3cc(N4CCC(c5c(C)ccnc5C5CC5(F)F)CC4)nc(C(F)(F)F)n3)C2C)CC1]

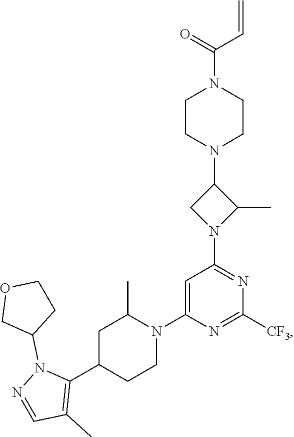 [SMILES: C=CC(=O)N1CCN(C2CN(c3cc(N4CCC(c5c(C)cnn5C5CCOC5)CC4C)nc(C(F)(F)F)n3)C2C)CC1]

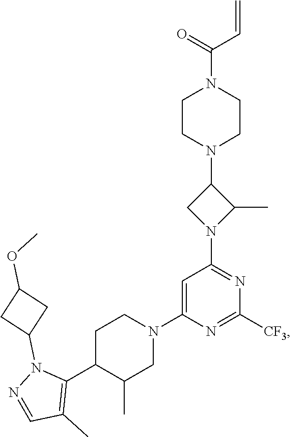 [SMILES: C=CC(=O)N1CCN(C2CN(c3cc(N4CCC(c5c(C)cnn5C5CC(OC)C5)C(C)C4)nc(C(F)(F)F)n3)C2C)CC1]

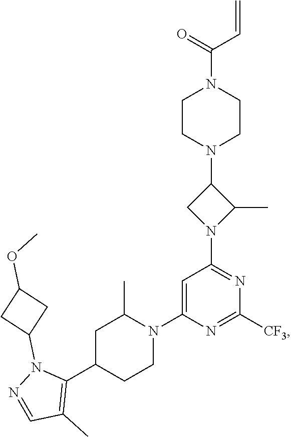 [SMILES: C=CC(=O)N1CCN(C2CN(c3cc(N4CCC(c5c(C)cnn5C5CC(OC)C5)CC4C)nc(C(F)(F)F)n3)C2C)CC1]

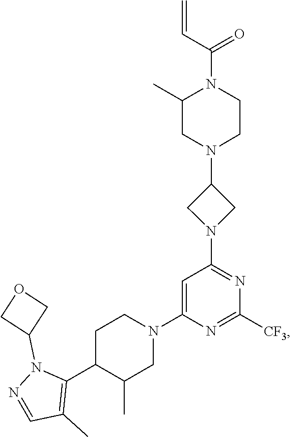 [SMILES: C=CC(=O)N1CCN(C2CN(c3cc(N4CCC(c5c(C)cnn5C5COC5)C(C)C4)nc(C(F)(F)F)n3)C2)CC1C]

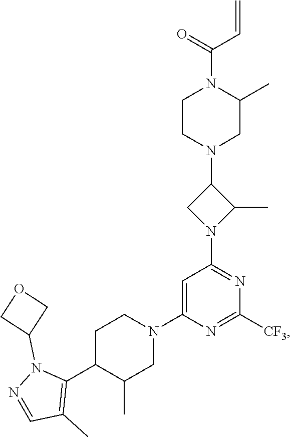 [SMILES: C=CC(=O)N1CCN(C2CN(c3cc(N4CCC(c5c(C)cnn5C5COC5)C(C)C4)nc(C(F)(F)F)n3)C2C)CC1C]

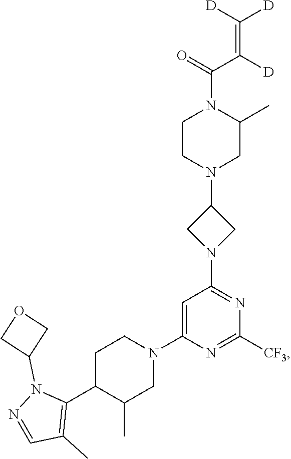 [SMILES: [2H]C([2H])=C([2H])C(=O)N1CCN(C2CN(c3cc(N4CCC(c5c(C)cnn5C5COC5)C(C)C4)nc(C(F)(F)F)n3)C2)CC1C]

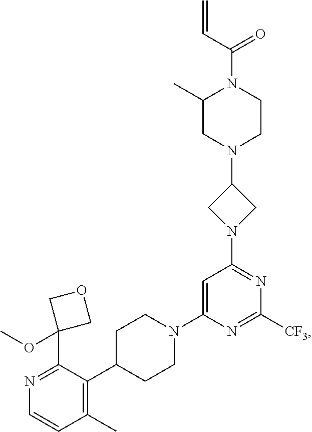 [SMILES: C=CC(=O)N1CCN(C2CN(c3cc(N4CCC(c5c(C)ccnc5C5(OC)COC5)CC4)nc(C(F)(F)F)n3)C2)CC1C]

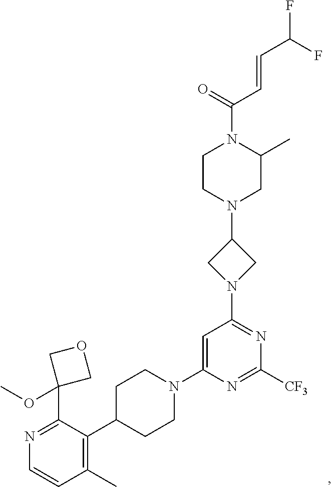 [SMILES: COC1(c2nccc(C)c2C2CCN(c3cc(N4CC(N5CCN(C(=O)/C=C/C(F)F)C(C)C5)C4)nc(C(F)(F)F)n3)CC2)COC1]

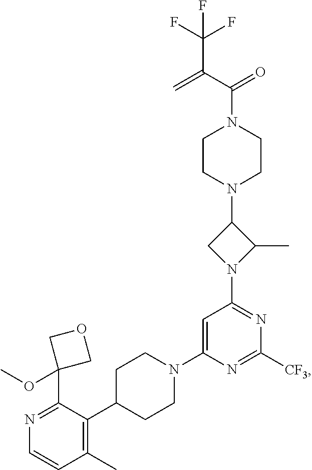 [SMILES: C=C(C(=O)N1CCN(C2CN(c3cc(N4CCC(c5c(C)ccnc5C5(OC)COC5)CC4)nc(C(F)(F)F)n3)C2C)CC1)C(F)(F)F]

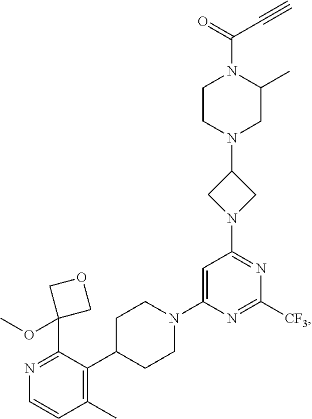 [SMILES: C#CC(=O)N1CCN(C2CN(c3cc(N4CCC(c5c(C)ccnc5C5(OC)COC5)CC4)nc(C(F)(F)F)n3)C2)CC1C]

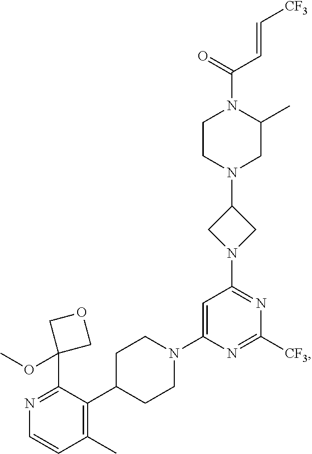 [SMILES: COC1(c2nccc(C)c2C2CCN(c3cc(N4CC(N5CCN(C(=O)/C=C/C(F)(F)F)C(C)C5)C4)nc(C(F)(F)F)n3)CC2)COC1]

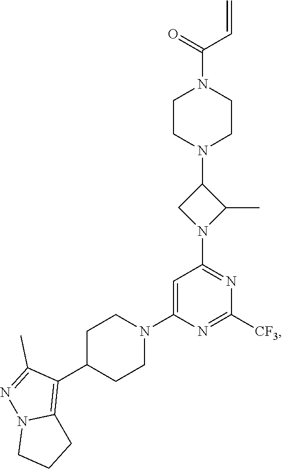 [SMILES: C=CC(=O)N1CCN(C2CN(c3cc(N4CCC(c5c(C)nn6c5CCC6)CC4)nc(C(F)(F)F)n3)C2C)CC1]

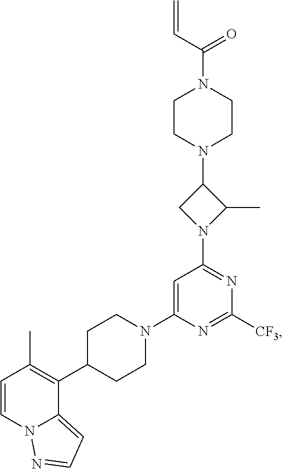 [SMILES: C=CC(=O)N1CCN(C2CN(c3cc(N4CCC(c5c(C)ccn6nccc56)CC4)nc(C(F)(F)F)n3)C2C)CC1]

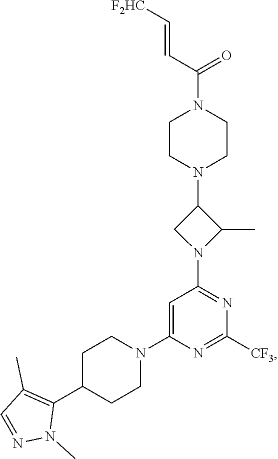 [SMILES: Cc1cnn(C)c1C1CCN(c2cc(N3CC(N4CCN(C(=O)/C=C/C(F)F)CC4)C3C)nc(C(F)(F)F)n2)CC1]

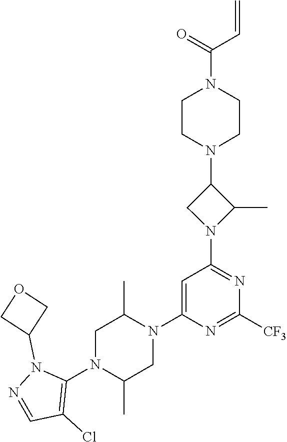 [SMILES: C=CC(=O)N1CCN(C2CN(c3cc(N4CC(C)N(c5c(Cl)cnn5C5COC5)CC4C)nc(C(F)(F)F)n3)C2C)CC1]

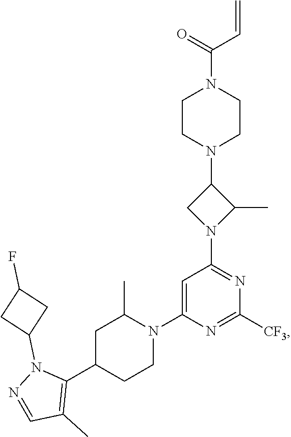 [SMILES: C=CC(=O)N1CCN(C2CN(c3cc(N4CCC(c5c(C)cnn5C5CC(F)C5)CC4C)nc(C(F)(F)F)n3)C2C)CC1]

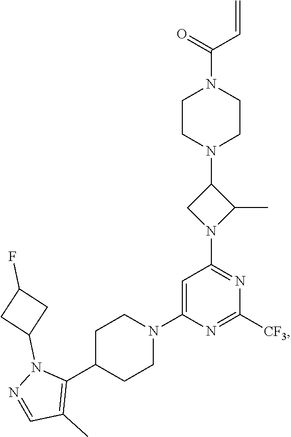 [SMILES: C=CC(=O)N1CCN(C2CN(c3cc(N4CCC(c5c(C)cnn5C5CC(F)C5)CC4)nc(C(F)(F)F)n3)C2C)CC1]

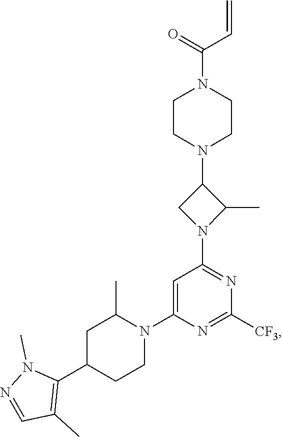 [SMILES: C=CC(=O)N1CCN(C2CN(c3cc(N4CCC(c5c(C)cnn5C)CC4C)nc(C(F)(F)F)n3)C2C)CC1]

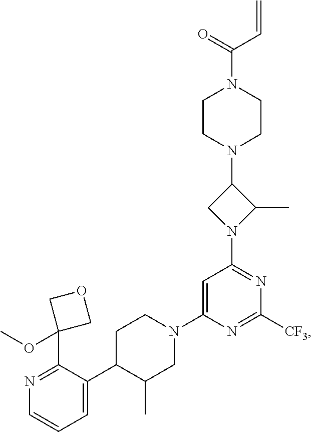 [SMILES: C=CC(=O)N1CCN(C2CN(c3cc(N4CCC(c5cccnc5C5(OC)COC5)C(C)C4)nc(C(F)(F)F)n3)C2C)CC1]

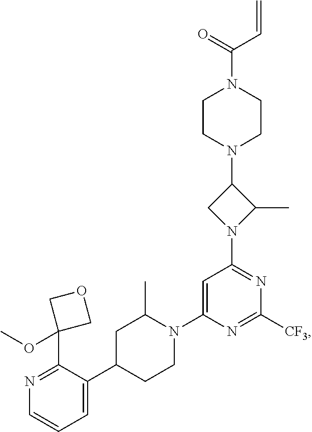 [SMILES: C=CC(=O)N1CCN(C2CN(c3cc(N4CCC(c5cccnc5C5(OC)COC5)CC4C)nc(C(F)(F)F)n3)C2C)CC1]

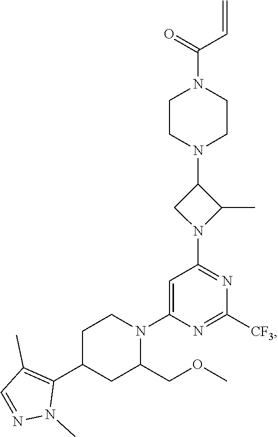 [SMILES: C=CC(=O)N1CCN(C2CN(c3cc(N4CCC(c5c(C)cnn5C)CC4COC)nc(C(F)(F)F)n3)C2C)CC1]

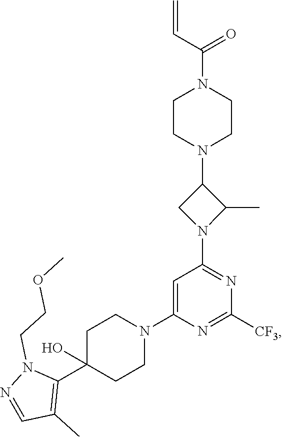 [SMILES: C=CC(=O)N1CCN(C2CN(c3cc(N4CCC(O)(c5c(C)cnn5CCOC)CC4)nc(C(F)(F)F)n3)C2C)CC1]

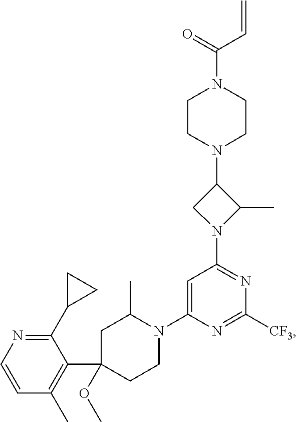 [SMILES: C=CC(=O)N1CCN(C2CN(c3cc(N4CCC(OC)(c5c(C)ccnc5C5CC5)CC4C)nc(C(F)(F)F)n3)C2C)CC1]

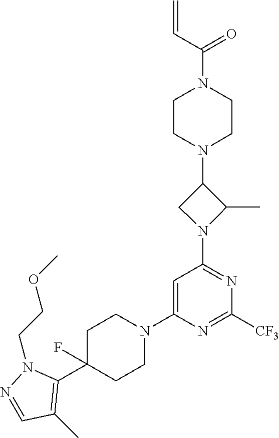 [SMILES: C=CC(=O)N1CCN(C2CN(c3cc(N4CCC(F)(c5c(C)cnn5CCOC)CC4)nc(C(F)(F)F)n3)C2C)CC1]

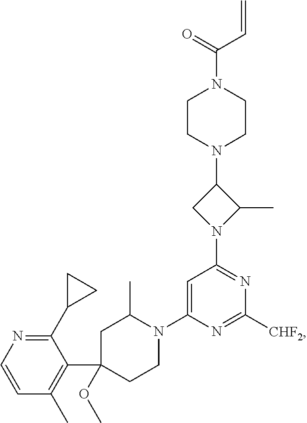 [SMILES: C=CC(=O)N1CCN(C2CN(c3cc(N4CCC(OC)(c5c(C)ccnc5C5CC5)CC4C)nc(C(F)F)n3)C2C)CC1]

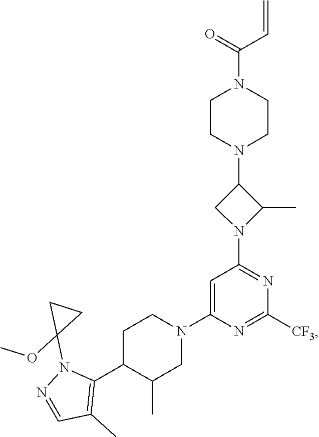 [SMILES: C=CC(=O)N1CCN(C2CN(c3cc(N4CCC(c5c(C)cnn5C5(OC)CC5)C(C)C4)nc(C(F)(F)F)n3)C2C)CC1]

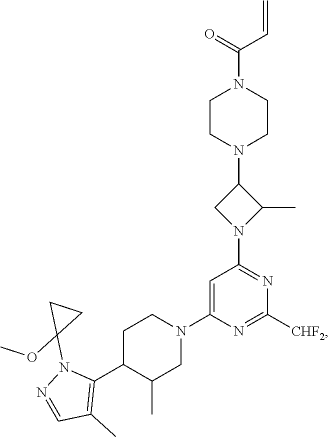 [SMILES: C=CC(=O)N1CCN(C2CN(c3cc(N4CCC(c5c(C)cnn5C5(OC)CC5)C(C)C4)nc(C(F)F)n3)C2C)CC1]

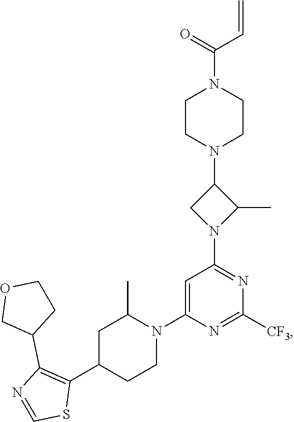 [SMILES: C=CC(=O)N1CCN(C2CN(c3cc(N4CCC(c5scnc5C5CCOC5)CC4C)nc(C(F)(F)F)n3)C2C)CC1]

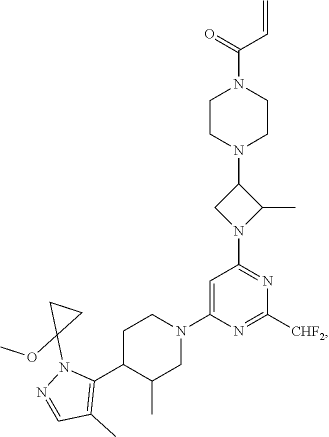 [SMILES: C=CC(=O)N1CCN(C2CN(c3cc(N4CCC(c5c(C)cnn5C5(OC)CC5)C(C)C4)nc(C(F)F)n3)C2C)CC1]

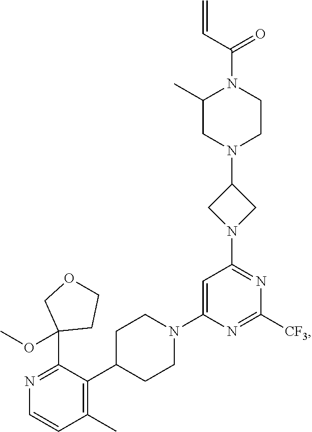

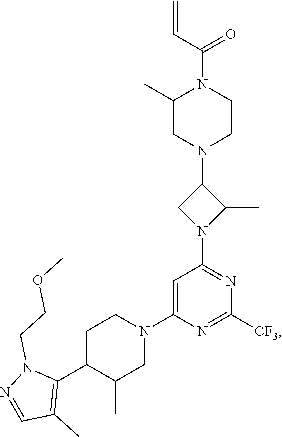 [SMILES: C=CC(=O)N1CCN(C2CN(c3cc(N4CCC(c5c(C)cnn5CCOC)C(C)C4)nc(C(F)(F)F)n3)C2C)CC1C]

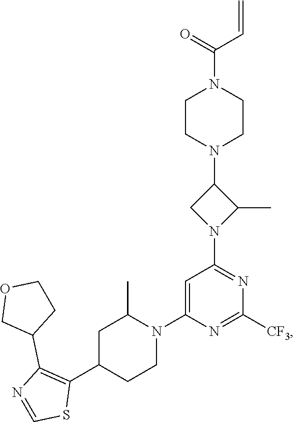 [SMILES: C=CC(=O)N1CCN(C2CN(c3cc(N4CCC(c5scnc5C5CCOC5)CC4C)nc(C(F)(F)F)n3)C2C)CC1]

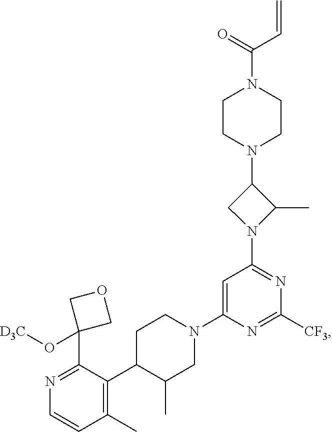 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2C)COC1]

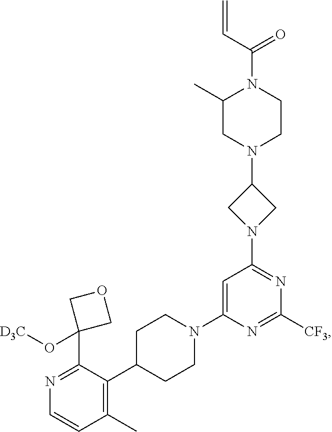 [SMILES: [2H]C([2H])([2H])OC1(c2nccc(C)c2C2CCN(c3cc(N4CC(N5CCN(C(=O)C=C)C(C)C5)C4)nc(C(F)(F)F)n3)CC2)COC1]

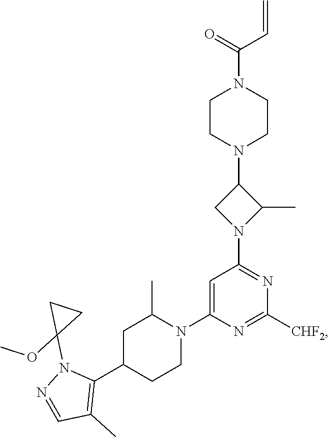 [SMILES: C=CC(=O)N1CCN(C2CN(c3cc(N4CCC(c5c(C)cnn5C5(OC)CC5)CC4C)nc(C(F)F)n3)C2C)CC1]

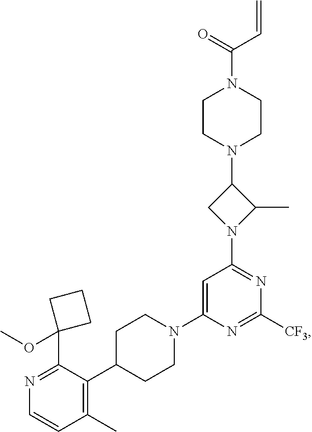 [SMILES: C=CC(=O)N1CCN(C2CN(c3cc(N4CCC(c5c(C)ccnc5C5(OC)CCC5)CC4)nc(C(F)(F)F)n3)C2C)CC1]

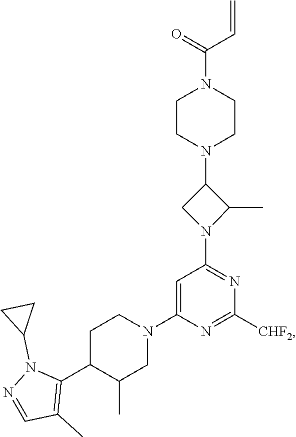 [SMILES: C=CC(=O)N1CCN(C2CN(c3cc(N4CCC(c5c(C)cnn5C5CC5)C(C)C4)nc(C(F)F)n3)C2C)CC1]

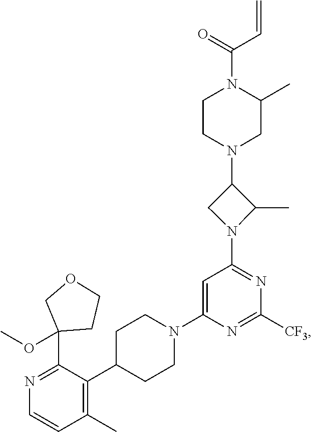 [SMILES: C=CC(=O)N1CCN(C2CN(c3cc(N4CCC(c5c(C)ccnc5C5(OC)CCOC5)CC4)nc(C(F)(F)F)n3)C2C)CC1C]

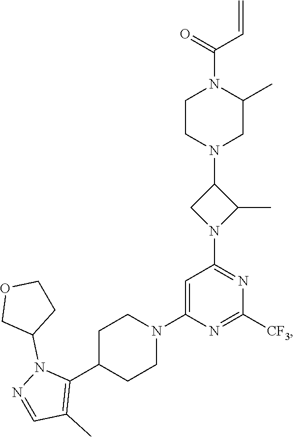 [SMILES: C=CC(=O)N1CCN(C2CN(c3cc(N4CCC(c5c(C)cnn5C5CCOC5)CC4)nc(C(F)(F)F)n3)C2C)CC1C]

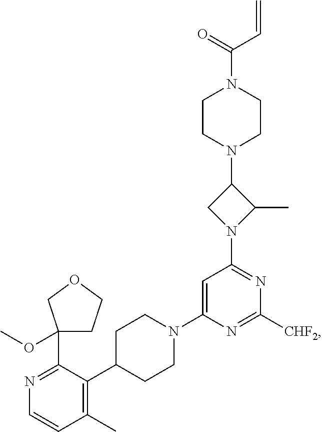 [SMILES: C=CC(=O)N1CCN(C2CN(c3cc(N4CCC(c5c(C)ccnc5C5(OC)CCOC5)CC4)nc(C(F)F)n3)C2C)CC1]

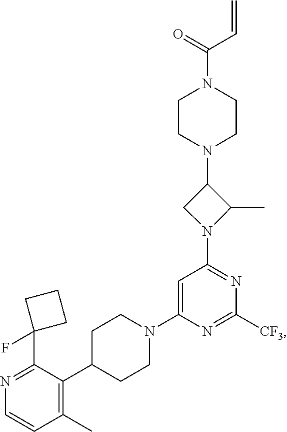 [SMILES: C=CC(=O)N1CCN(C2CN(c3cc(N4CCC(c5c(C)ccnc5C5(F)CCC5)CC4)nc(C(F)(F)F)n3)C2C)CC1]

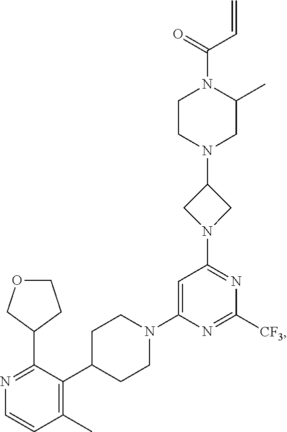 [SMILES: C=CC(=O)N1CCN(C2CN(c3cc(N4CCC(c5c(C)ccnc5C5CCOC5)CC4)nc(C(F)(F)F)n3)C2)CC1C]

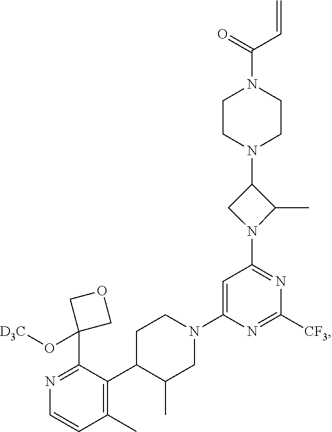 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2C)COC1]

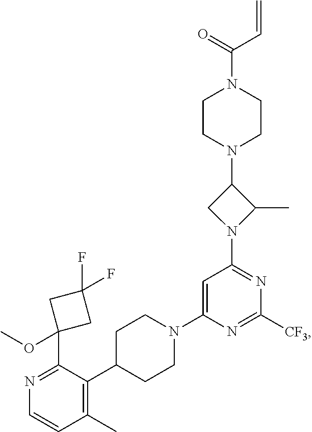 [SMILES: C=CC(=O)N1CCN(C2CN(c3cc(N4CCC(c5c(C)ccnc5C5(OC)CC(F)(F)C5)CC4)nc(C(F)(F)F)n3)C2C)CC1]

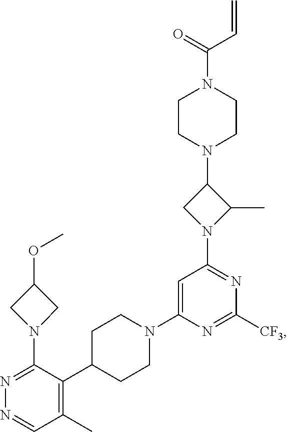 [SMILES: C=CC(=O)N1CCN(C2CN(c3cc(N4CCC(c5c(C)cnnc5N5CC(OC)C5)CC4)nc(C(F)(F)F)n3)C2C)CC1]

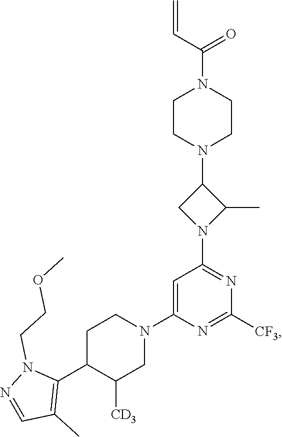 [SMILES: [2H]C([2H])([2H])C1CN(c2cc(N3CC(N4CCN(C(=O)C=C)CC4)C3C)nc(C(F)(F)F)n2)CCC1c1c(C)cnn1CCOC]

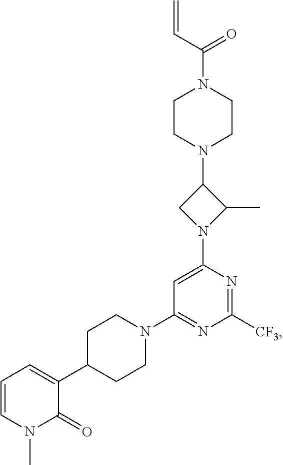 [SMILES: C=CC(=O)N1CCN(C2CN(c3cc(N4CCC(c5cccn(C)c5=O)CC4)nc(C(F)(F)F)n3)C2C)CC1]

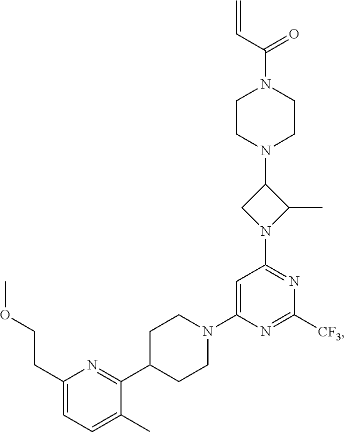 [SMILES: C=CC(=O)N1CCN(C2CN(c3cc(N4CCC(c5nc(CCOC)ccc5C)CC4)nc(C(F)(F)F)n3)C2C)CC1]

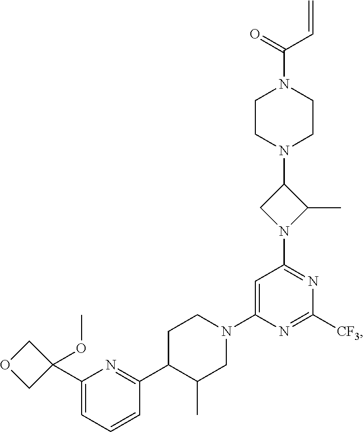 [SMILES: C=CC(=O)N1CCN(C2CN(c3cc(N4CCC(c5cccc(C6(OC)COC6)n5)C(C)C4)nc(C(F)(F)F)n3)C2C)CC1]

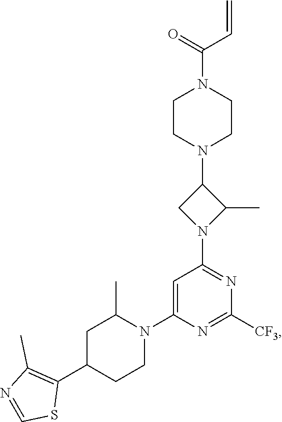 [SMILES: C=CC(=O)N1CCN(C2CN(c3cc(N4CCC(c5scnc5C)CC4C)nc(C(F)(F)F)n3)C2C)CC1]

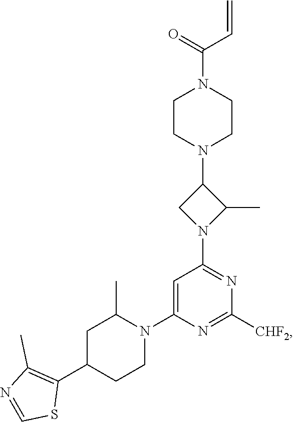 [SMILES: C=CC(=O)N1CCN(C2CN(c3cc(N4CCC(c5scnc5C)CC4C)nc(C(F)F)n3)C2C)CC1]

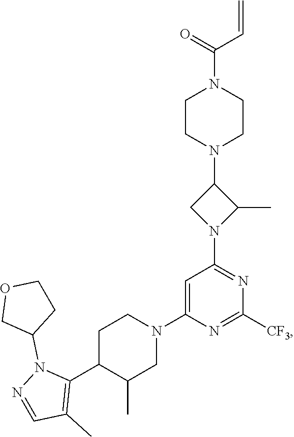 [SMILES: C=CC(=O)N1CCN(C2CN(c3cc(N4CCC(c5c(C)cnn5C5CCOC5)C(C)C4)nc(C(F)(F)F)n3)C2C)CC1]

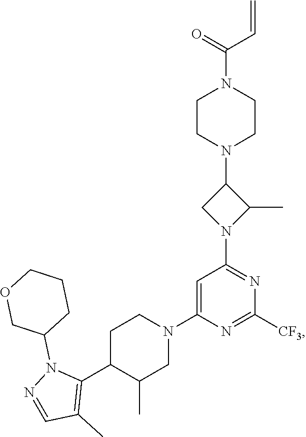 [SMILES: C=CC(=O)N1CCN(C2CN(c3cc(N4CCC(c5c(C)cnn5C5CCCOC5)C(C)C4)nc(C(F)(F)F)n3)C2C)CC1]

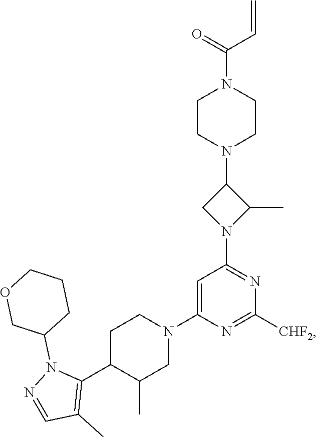 [SMILES: C=CC(=O)N1CCN(C2CN(c3cc(N4CCC(c5c(C)cnn5C5CCCOC5)C(C)C4)nc(C(F)F)n3)C2C)CC1]

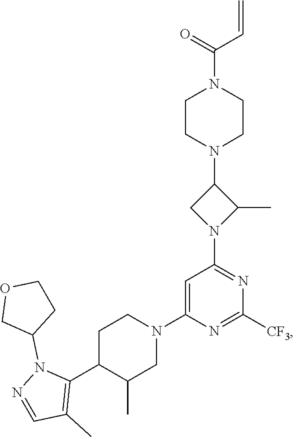 [SMILES: C=CC(=O)N1CCN(C2CN(c3cc(N4CCC(c5c(C)cnn5C5CCOC5)C(C)C4)nc(C(F)(F)F)n3)C2C)CC1]

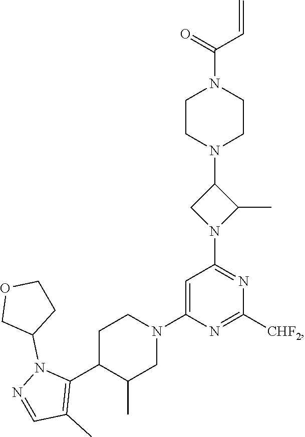 [SMILES: C=CC(=O)N1CCN(C2CN(c3cc(N4CCC(c5c(C)cnn5C5CCOC5)C(C)C4)nc(C(F)F)n3)C2C)CC1]

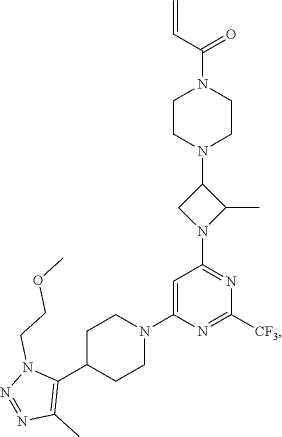 [SMILES: C=CC(=O)N1CCN(C2CN(c3cc(N4CCC(c5c(C)nnn5CCOC)CC4)nc(C(F)(F)F)n3)C2C)CC1]

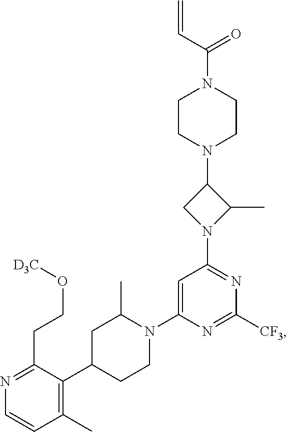 [SMILES: [2H]C([2H])([2H])OCCc1nccc(C)c1C1CCN(c2cc(N3CC(N4CCN(C(=O)C=C)CC4)C3C)nc(C(F)(F)F)n2)C(C)C1]

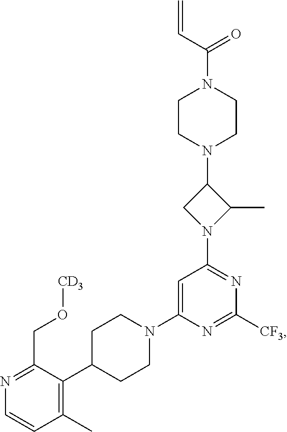 [SMILES: [2H]C([2H])([2H])OCc1nccc(C)c1C1CCN(c2cc(N3CC(N4CCN(C(=O)C=C)CC4)C3C)nc(C(F)(F)F)n2)CC1]

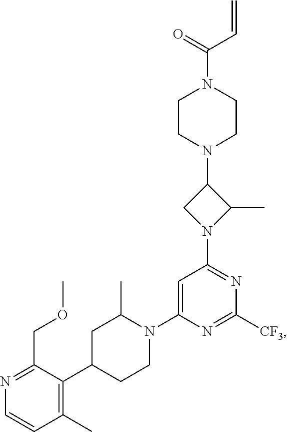 [SMILES: C=CC(=O)N1CCN(C2CN(c3cc(N4CCC(c5c(C)ccnc5COC)CC4C)nc(C(F)(F)F)n3)C2C)CC1]

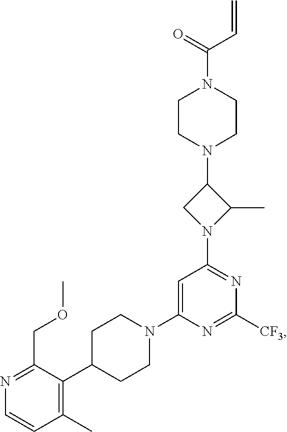 [SMILES: C=CC(=O)N1CCN(C2CN(c3cc(N4CCC(c5c(C)ccnc5COC)CC4)nc(C(F)(F)F)n3)C2C)CC1]

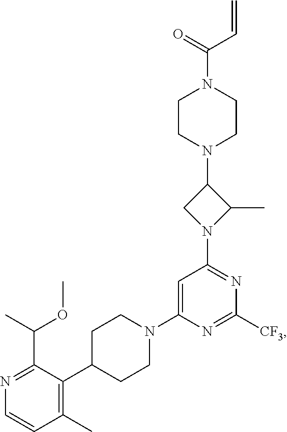 [SMILES: C=CC(=O)N1CCN(C2CN(c3cc(N4CCC(c5c(C)ccnc5C(C)OC)CC4)nc(C(F)(F)F)n3)C2C)CC1]

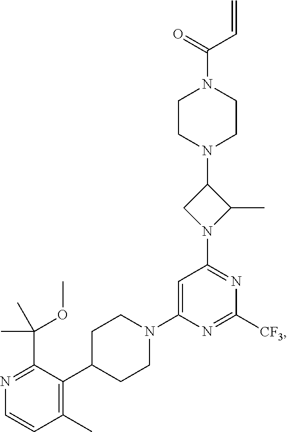 [SMILES: C=CC(=O)N1CCN(C2CN(c3cc(N4CCC(c5c(C)ccnc5C(C)(C)OC)CC4)nc(C(F)(F)F)n3)C2C)CC1]

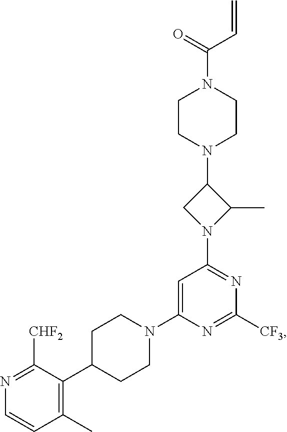 [SMILES: C=CC(=O)N1CCN(C2CN(c3cc(N4CCC(c5c(C)ccnc5C(F)F)CC4)nc(C(F)(F)F)n3)C2C)CC1]

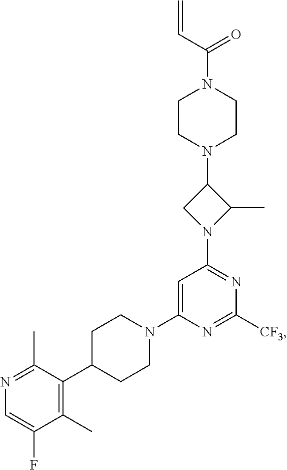 [SMILES: C=CC(=O)N1CCN(C2CN(c3cc(N4CCC(c5c(C)ncc(F)c5C)CC4)nc(C(F)(F)F)n3)C2C)CC1]

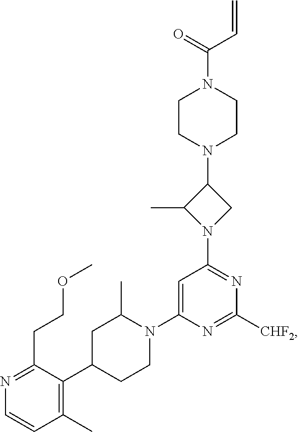 [SMILES: C=CC(=O)N1CCN(C2CN(c3cc(N4CCC(c5c(C)ccnc5CCOC)CC4C)nc(C(F)F)n3)C2C)CC1]

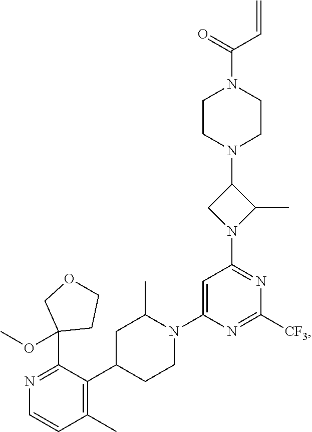 [SMILES: C=CC(=O)N1CCN(C2CN(c3cc(N4CCC(c5c(C)ccnc5C5(OC)CCOC5)CC4C)nc(C(F)(F)F)n3)C2C)CC1]

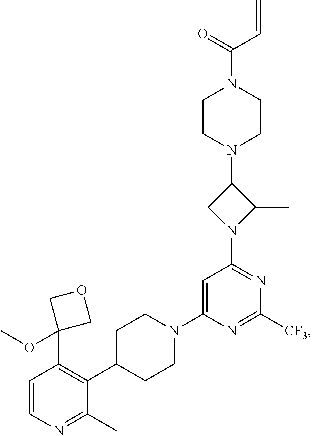 [SMILES: C=CC(=O)N1CCN(C2CN(c3cc(N4CCC(c5c(C6(OC)COC6)ccnc5C)CC4)nc(C(F)(F)F)n3)C2C)CC1]

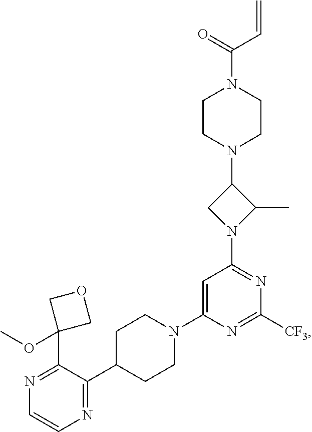 [SMILES: C=CC(=O)N1CCN(C2CN(c3cc(N4CCC(c5nccnc5C5(OC)COC5)CC4)nc(C(F)(F)F)n3)C2C)CC1]

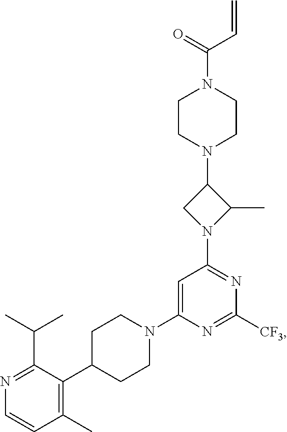 [SMILES: C=CC(=O)N1CCN(C2CN(c3cc(N4CCC(c5c(C)ccnc5C(C)C)CC4)nc(C(F)(F)F)n3)C2C)CC1]

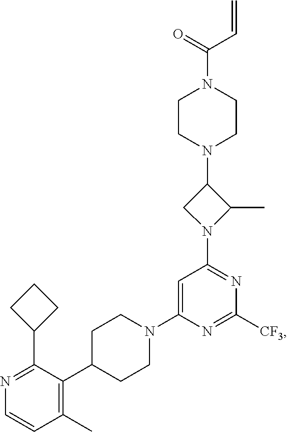 [SMILES: C=CC(=O)N1CCN(C2CN(c3cc(N4CCC(c5c(C)ccnc5C5CCC5)CC4)nc(C(F)(F)F)n3)C2C)CC1]

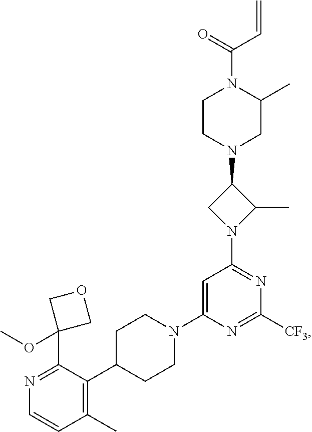 [SMILES: C=CC(=O)N1CCN([C@@H]2CN(c3cc(N4CCC(c5c(C)ccnc5C5(OC)COC5)CC4)nc(C(F)(F)F)n3)C2C)CC1C]

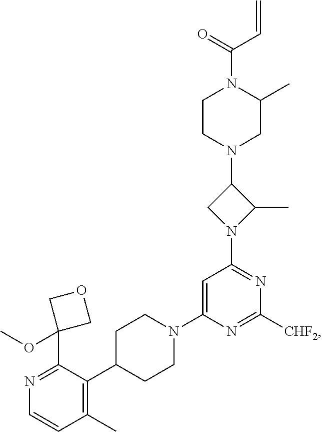 [SMILES: C=CC(=O)N1CCN(C2CN(c3cc(N4CCC(c5c(C)ccnc5C5(OC)COC5)CC4)nc(C(F)F)n3)C2C)CC1C]

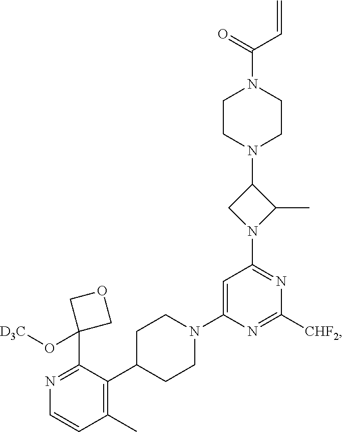 [SMILES: [2H]C([2H])([2H])OC1(c2nccc(C)c2C2CCN(c3cc(N4CC(N5CCN(C(=O)C=C)CC5)C4C)nc(C(F)F)n3)CC2)COC1]

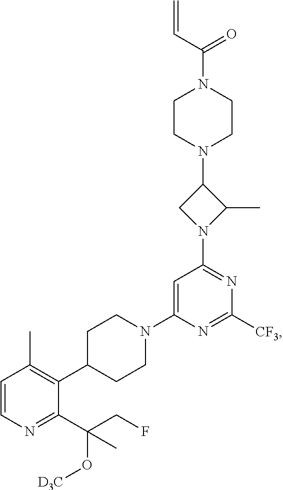 [SMILES: [2H]C([2H])([2H])OC(C)(CF)c1nccc(C)c1C1CCN(c2cc(N3CC(N4CCN(C(=O)C=C)CC4)C3C)nc(C(F)(F)F)n2)CC1]

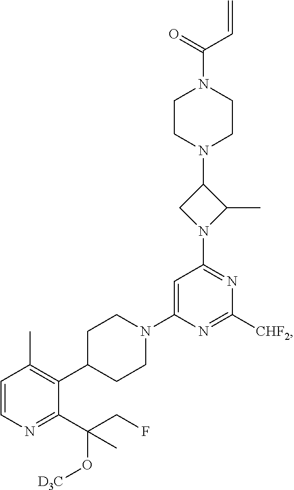 [SMILES: [2H]C([2H])([2H])OC(C)(CF)c1nccc(C)c1C1CCN(c2cc(N3CC(N4CCN(C(=O)C=C)CC4)C3C)nc(C(F)F)n2)CC1]

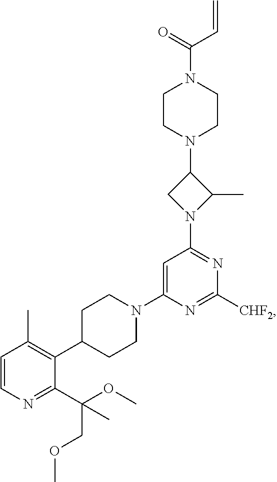 [SMILES: C=CC(=O)N1CCN(C2CN(c3cc(N4CCC(c5c(C)ccnc5C(C)(COC)OC)CC4)nc(C(F)F)n3)C2C)CC1]

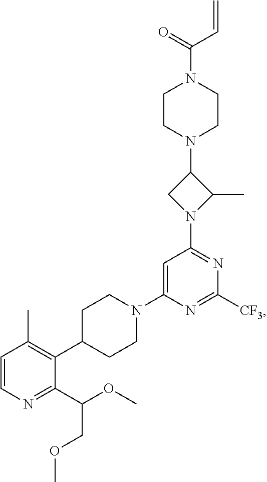 [SMILES: C=CC(=O)N1CCN(C2CN(c3cc(N4CCC(c5c(C)ccnc5C(COC)OC)CC4)nc(C(F)(F)F)n3)C2C)CC1]

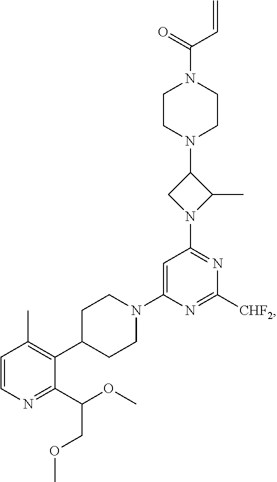 [SMILES: C=CC(=O)N1CCN(C2CN(c3cc(N4CCC(c5c(C)ccnc5C(COC)OC)CC4)nc(C(F)F)n3)C2C)CC1]

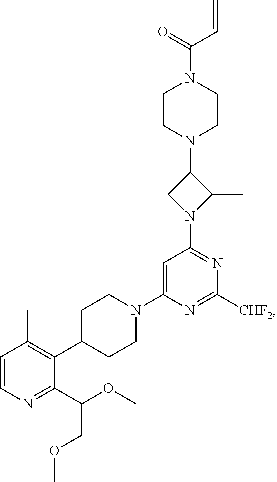 [SMILES: C=CC(=O)N1CCN(C2CN(c3cc(N4CCC(c5c(C)ccnc5C(COC)OC)CC4)nc(C(F)F)n3)C2C)CC1]

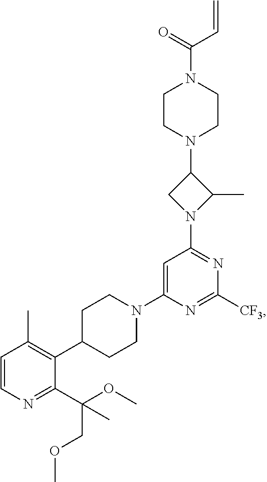 [SMILES: C=CC(=O)N1CCN(C2CN(c3cc(N4CCC(c5c(C)ccnc5C(C)(COC)OC)CC4)nc(C(F)(F)F)n3)C2C)CC1]

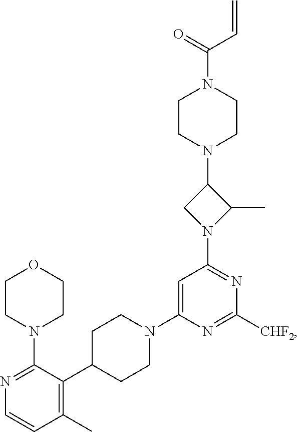 [SMILES: C=CC(=O)N1CCN(C2CN(c3cc(N4CCC(c5c(C)ccnc5N5CCOCC5)CC4)nc(C(F)F)n3)C2C)CC1]

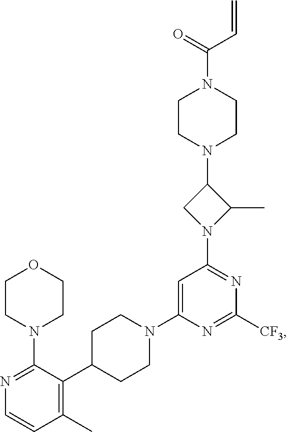 [SMILES: C=CC(=O)N1CCN(C2CN(c3cc(N4CCC(c5c(C)ccnc5N5CCOCC5)CC4)nc(C(F)(F)F)n3)C2C)CC1]

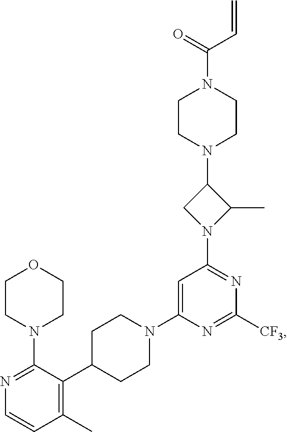 [SMILES: C=CC(=O)N1CCN(C2CN(c3cc(N4CCC(c5c(C)ccnc5N5CCOCC5)CC4)nc(C(F)(F)F)n3)C2C)CC1]

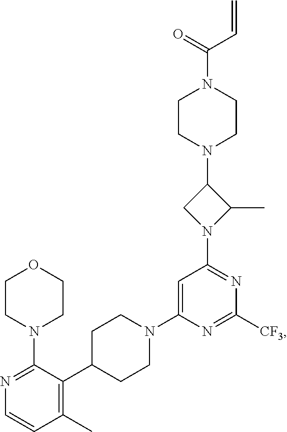 [SMILES: C=CC(=O)N1CCN(C2CN(c3cc(N4CCC(c5c(C)ccnc5N5CCOCC5)CC4)nc(C(F)(F)F)n3)C2C)CC1]

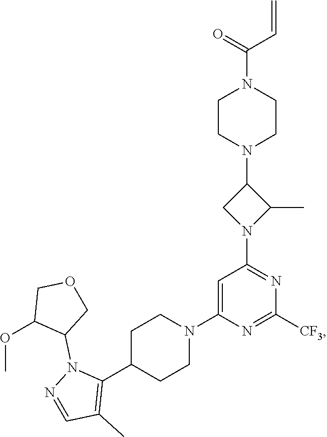 [SMILES: C=CC(=O)N1CCN(C2CN(c3cc(N4CCC(c5c(C)cnn5C5COCC5OC)CC4)nc(C(F)(F)F)n3)C2C)CC1]

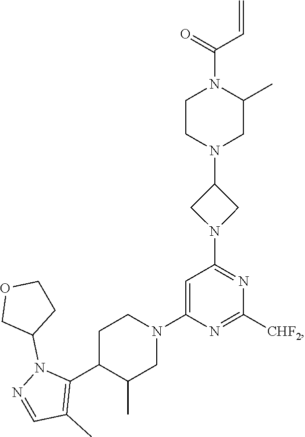 [SMILES: C=CC(=O)N1CCN(C2CN(c3cc(N4CCC(c5c(C)cnn5C5CCOC5)C(C)C4)nc(C(F)F)n3)C2)CC1C]

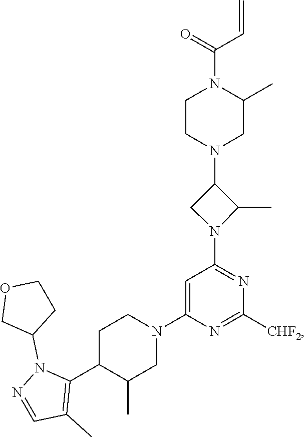 [SMILES: C=CC(=O)N1CCN(C2CN(c3cc(N4CCC(c5c(C)cnn5C5CCOC5)C(C)C4)nc(C(F)F)n3)C2C)CC1C]

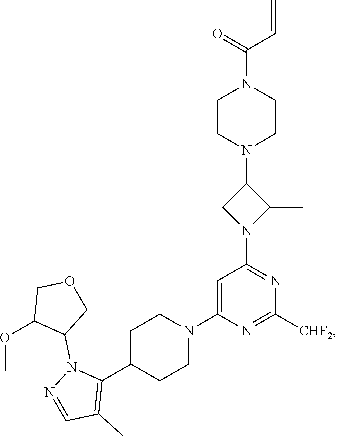 [SMILES: C=CC(=O)N1CCN(C2CN(c3cc(N4CCC(c5c(C)cnn5C5COCC5OC)CC4)nc(C(F)F)n3)C2C)CC1]

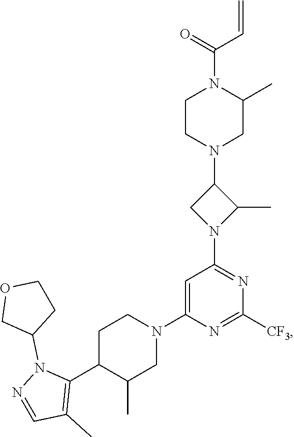 [SMILES: C=CC(=O)N1CCN(C2CN(c3cc(N4CCC(c5c(C)cnn5C5CCOC5)C(C)C4)nc(C(F)(F)F)n3)C2C)CC1C]

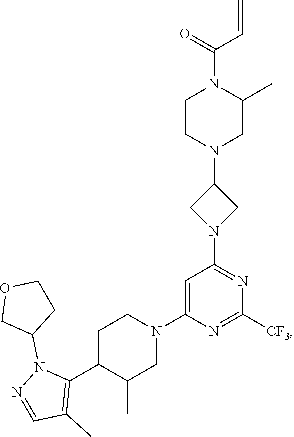 [SMILES: C=CC(=O)N1CCN(C2CN(c3cc(N4CCC(c5c(C)cnn5C5CCOC5)C(C)C4)nc(C(F)(F)F)n3)C2)CC1C]

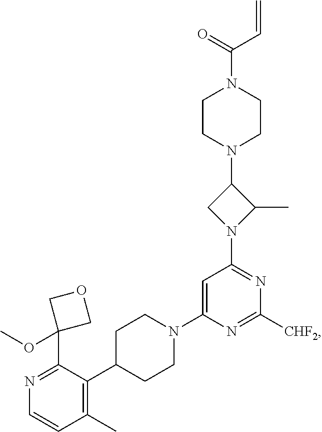 [SMILES: C=CC(=O)N1CCN(C2CN(c3cc(N4CCC(c5c(C)ccnc5C5(OC)COC5)CC4)nc(C(F)F)n3)C2C)CC1]

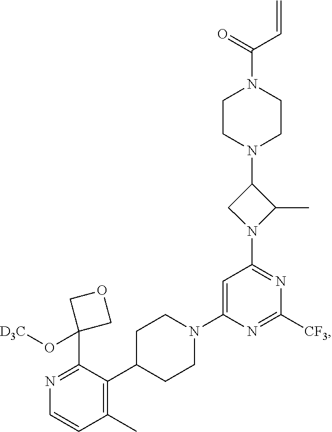 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2)COC1]

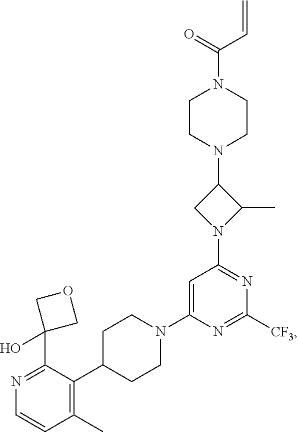 [SMILES: C=CC(=O)N1CCN(C2CN(c3cc(N4CCC(c5c(C)ccnc5C5(O)COC5)CC4)nc(C(F)(F)F)n3)C2C)CC1]

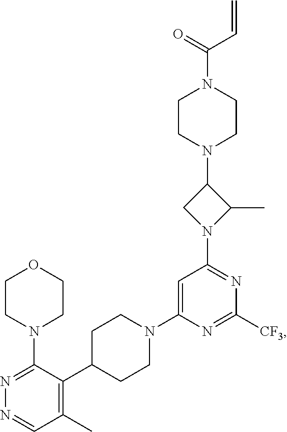 [SMILES: C=CC(=O)N1CCN(C2CN(c3cc(N4CCC(c5c(C)cnnc5N5CCOCC5)CC4)nc(C(F)(F)F)n3)C2C)CC1]

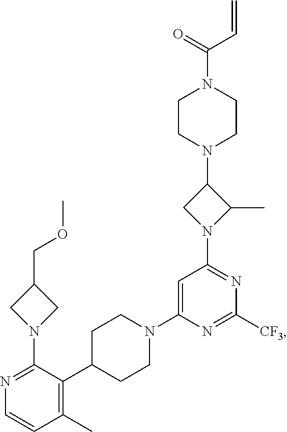 [SMILES: C=CC(=O)N1CCN(C2CN(c3cc(N4CCC(c5c(C)ccnc5N5CC(COC)C5)CC4)nc(C(F)(F)F)n3)C2C)CC1]

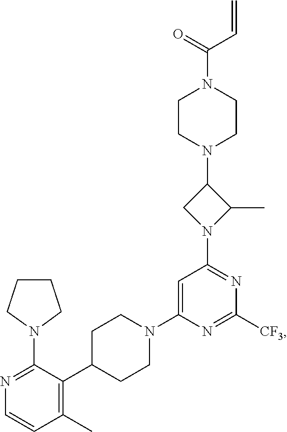 [SMILES: C=CC(=O)N1CCN(C2CN(c3cc(N4CCC(c5c(C)ccnc5N5CCCC5)CC4)nc(C(F)(F)F)n3)C2C)CC1]

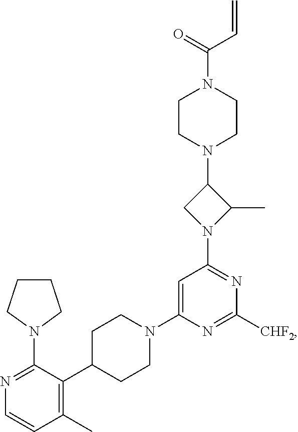 [SMILES: C=CC(=O)N1CCN(C2CN(c3cc(N4CCC(c5c(C)ccnc5N5CCCC5)CC4)nc(C(F)F)n3)C2C)CC1]

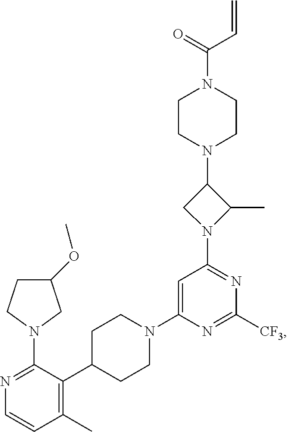 [SMILES: C=CC(=O)N1CCN(C2CN(c3cc(N4CCC(c5c(C)ccnc5N5CCC(OC)C5)CC4)nc(C(F)(F)F)n3)C2C)CC1]

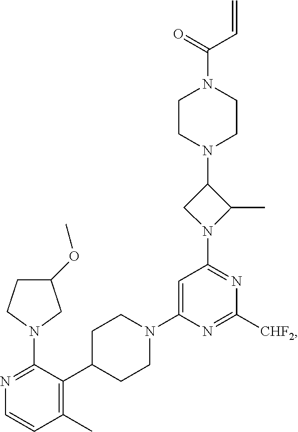 [SMILES: C=CC(=O)N1CCN(C2CN(c3cc(N4CCC(c5c(C)ccnc5N5CCC(OC)C5)CC4)nc(C(F)F)n3)C2C)CC1]

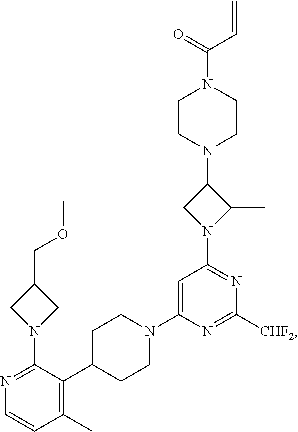 [SMILES: C=CC(=O)N1CCN(C2CN(c3cc(N4CCC(c5c(C)ccnc5N5CC(COC)C5)CC4)nc(C(F)F)n3)C2C)CC1]

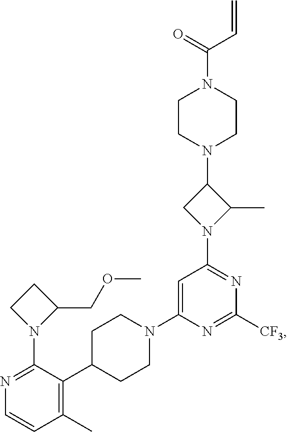 [SMILES: C=CC(=O)N1CCN(C2CN(c3cc(N4CCC(c5c(C)ccnc5N5CCC5COC)CC4)nc(C(F)(F)F)n3)C2C)CC1]

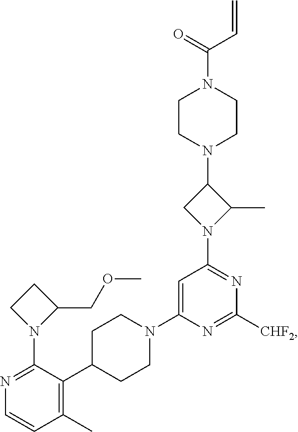 [SMILES: C=CC(=O)N1CCN(C2CN(c3cc(N4CCC(c5c(C)ccnc5N5CCC5COC)CC4)nc(C(F)F)n3)C2C)CC1]

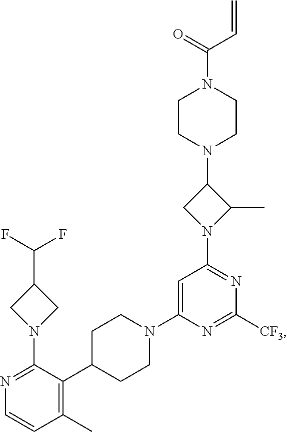 [SMILES: C=CC(=O)N1CCN(C2CN(c3cc(N4CCC(c5c(C)ccnc5N5CC(C(F)F)C5)CC4)nc(C(F)(F)F)n3)C2C)CC1]

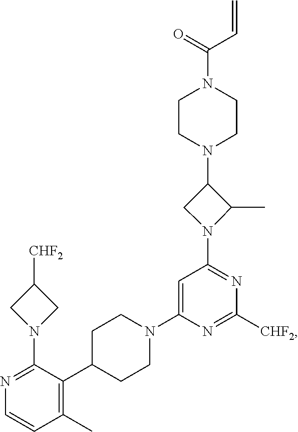 [SMILES: C=CC(=O)N1CCN(C2CN(c3cc(N4CCC(c5c(C)ccnc5N5CC(C(F)F)C5)CC4)nc(C(F)F)n3)C2C)CC1]

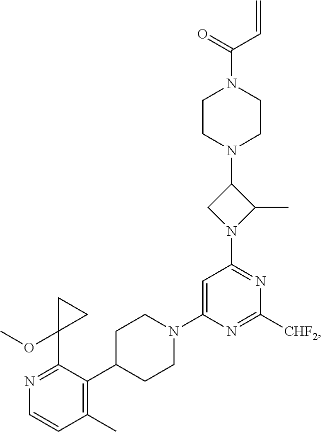 [SMILES: C=CC(=O)N1CCN(C2CN(c3cc(N4CCC(c5c(C)ccnc5C5(OC)CC5)CC4)nc(C(F)F)n3)C2C)CC1]

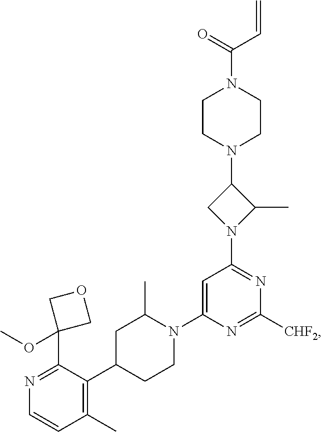 [SMILES: C=CC(=O)N1CCN(C2CN(c3cc(N4CCC(c5c(C)ccnc5C5(OC)COC5)CC4C)nc(C(F)F)n3)C2C)CC1]

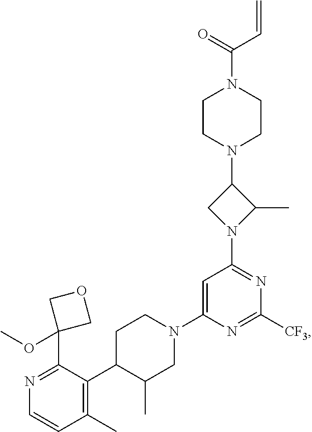 [SMILES: C=CC(=O)N1CCN(C2CN(c3cc(N4CCC(c5c(C)ccnc5C5(OC)COC5)C(C)C4)nc(C(F)(F)F)n3)C2C)CC1]

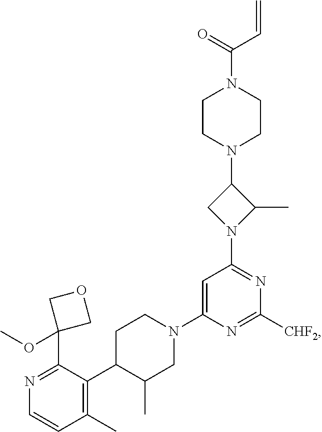 [SMILES: C=CC(=O)N1CCN(C2CN(c3cc(N4CCC(c5c(C)ccnc5C5(OC)COC5)C(C)C4)nc(C(F)F)n3)C2C)CC1]

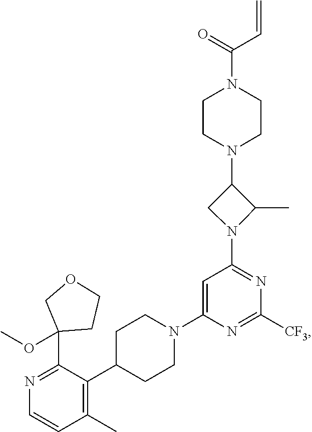 [SMILES: C=CC(=O)N1CCN(C2CN(c3cc(N4CCC(c5c(C)ccnc5C5(OC)CCOC5)CC4)nc(C(F)(F)F)n3)C2C)CC1]

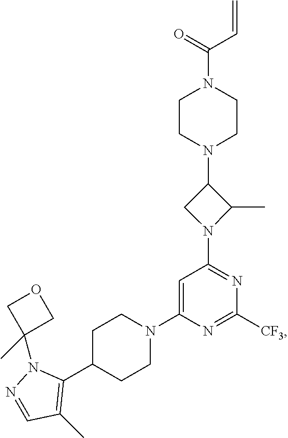 [SMILES: C=CC(=O)N1CCN(C2CN(c3cc(N4CCC(c5c(C)cnn5C5(C)COC5)CC4)nc(C(F)(F)F)n3)C2C)CC1]

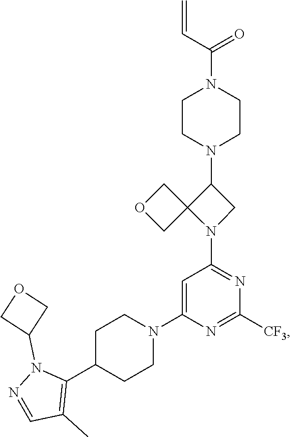 [SMILES: C=CC(=O)N1CCN(C2CN(c3cc(N4CCC(c5c(C)cnn5C5COC5)CC4)nc(C(F)(F)F)n3)C23COC3)CC1]

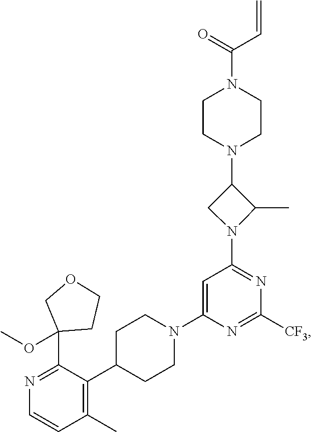 [SMILES: C=CC(=O)N1CCN(C2CN(c3cc(N4CCC(c5c(C)ccnc5C5(OC)CCOC5)CC4)nc(C(F)(F)F)n3)C2C)CC1]

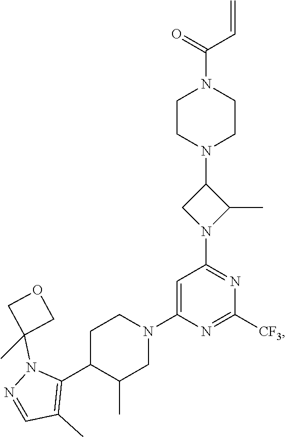 [SMILES: C=CC(=O)N1CCN(C2CN(c3cc(N4CCC(c5c(C)cnn5C5(C)COC5)C(C)C4)nc(C(F)(F)F)n3)C2C)CC1]

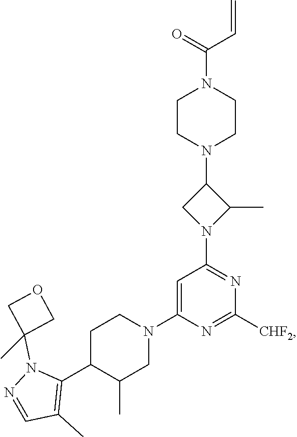 [SMILES: C=CC(=O)N1CCN(C2CN(c3cc(N4CCC(c5c(C)cnn5C5(C)COC5)C(C)C4)nc(C(F)F)n3)C2C)CC1]

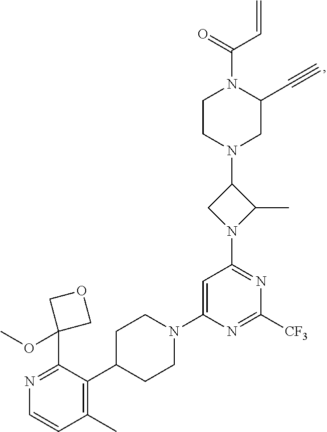 [SMILES: C#CC1CN(C2CN(c3cc(N4CCC(c5c(C)ccnc5C5(OC)COC5)CC4)nc(C(F)(F)F)n3)C2C)CCN1C(=O)C=C]

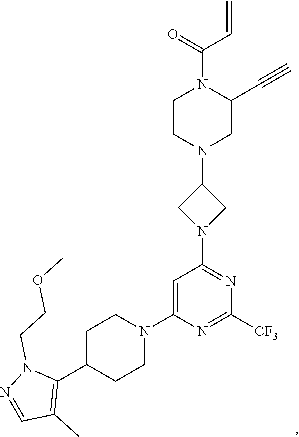 [SMILES: C#CC1CN(C2CN(c3cc(N4CCC(c5c(C)cnn5CCOC)CC4)nc(C(F)(F)F)n3)C2)CCN1C(=O)C=C]

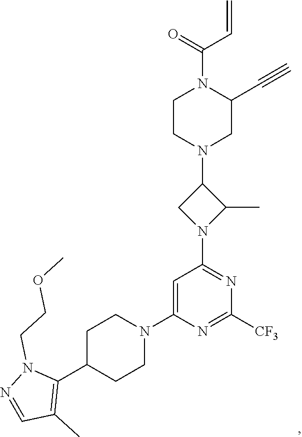 [SMILES: C#CC1CN(C2CN(c3cc(N4CCC(c5c(C)cnn5CCOC)CC4)nc(C(F)(F)F)n3)C2C)CCN1C(=O)C=C]

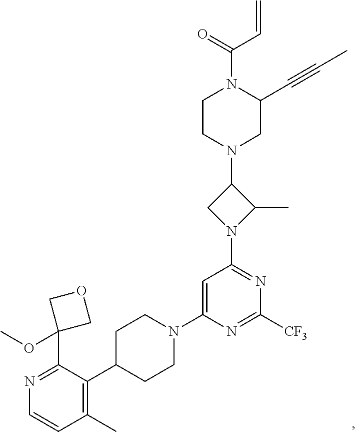 [SMILES: C=CC(=O)N1CCN(C2CN(c3cc(N4CCC(c5c(C)ccnc5C5(OC)COC5)CC4)nc(C(F)(F)F)n3)C2C)CC1C#CC]

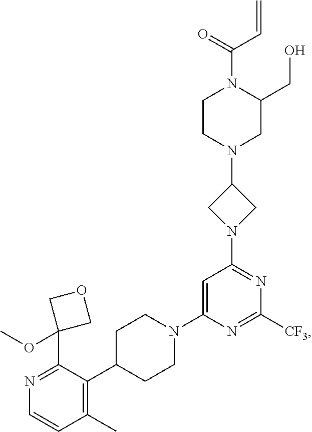 [SMILES: C=CC(=O)N1CCN(C2CN(c3cc(N4CCC(c5c(C)ccnc5C5(OC)COC5)CC4)nc(C(F)(F)F)n3)C2)CC1CO]

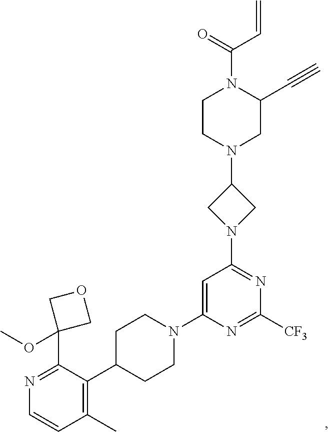 [SMILES: C#CC1CN(C2CN(c3cc(N4CCC(c5c(C)ccnc5C5(OC)COC5)CC4)nc(C(F)(F)F)n3)C2)CCN1C(=O)C=C]

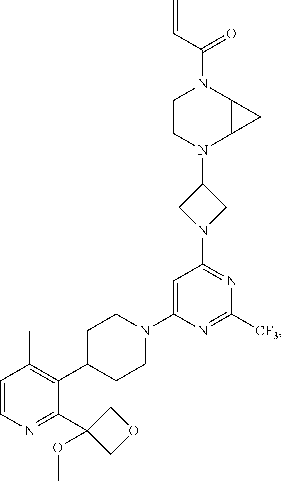 [SMILES: C=CC(=O)N1CCN(C2CN(c3cc(N4CCC(c5c(C)ccnc5C5(OC)COC5)CC4)nc(C(F)(F)F)n3)C2)C2CC21]

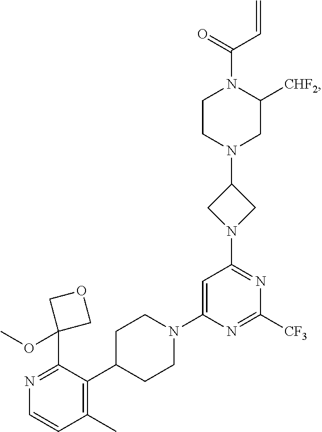 [SMILES: C=CC(=O)N1CCN(C2CN(c3cc(N4CCC(c5c(C)ccnc5C5(OC)COC5)CC4)nc(C(F)(F)F)n3)C2)CC1C(F)F]

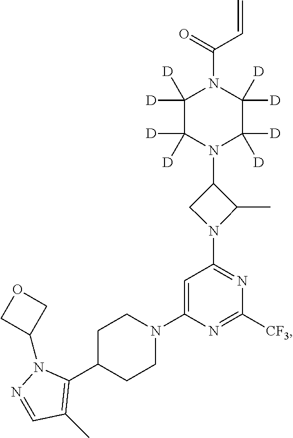 [SMILES: [2H]C1([2H])N(C(=O)C=C)C([2H])([2H])C([2H])([2H])N(C2CN(c3cc(N4CCC(c5c(C)cnn5C5COC5)CC4)nc(C(F)(F)F)n3)C2C)C1([2H])[2H]]

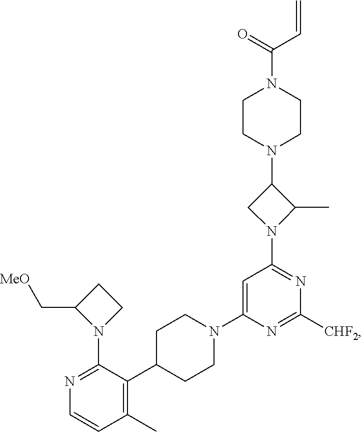 [SMILES: C=CC(=O)N1CCN(C2CN(c3cc(N4CCC(c5c(C)ccnc5N5CCC5COC)CC4)nc(C(F)F)n3)C2C)CC1]

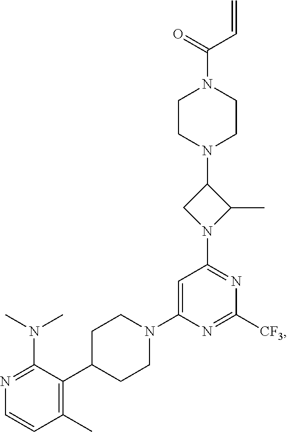 [SMILES: C=CC(=O)N1CCN(C2CN(c3cc(N4CCC(c5c(C)ccnc5N(C)C)CC4)nc(C(F)(F)F)n3)C2C)CC1]

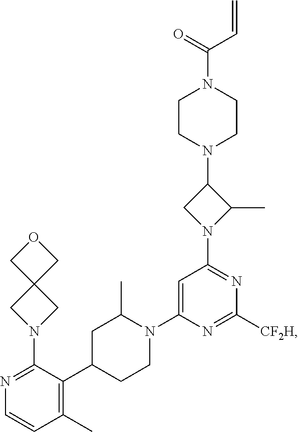 [SMILES: C=CC(=O)N1CCN(C2CN(c3cc(N4CCC(c5c(C)ccnc5N5CC6(COC6)C5)CC4C)nc(C(F)F)n3)C2C)CC1]

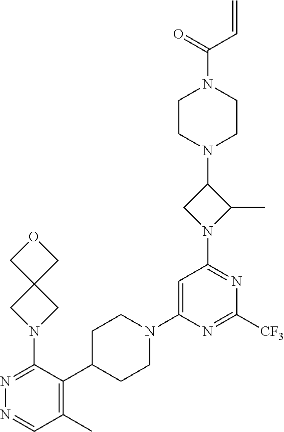 [SMILES: C=CC(=O)N1CCN(C2CN(c3cc(N4CCC(c5c(C)cnnc5N5CC6(COC6)C5)CC4)nc(C(F)(F)F)n3)C2C)CC1]

In some cases, the compound of Formula (I) is selected from the list of compounds in Table C, of a pharmaceutically acceptable salt thereof.

In some cases, A is N; X is

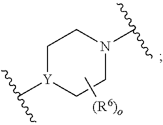

and Y is N, and the disclosure provides compounds of Formula (ID):

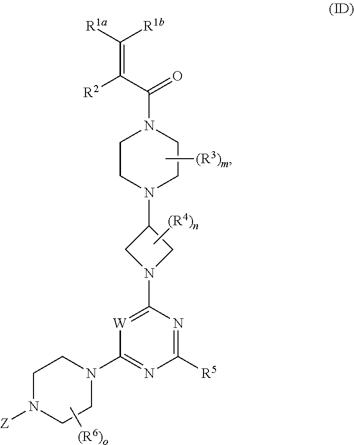

and pharmaceutically acceptable salts thereof, wherein the substituents are as previously defined herein, Contemplated compounds of Formula (ID) include, but are not limited to, those listed in Table D, belon, and pharmaceutically acceptable salts thereof.

TABLE D

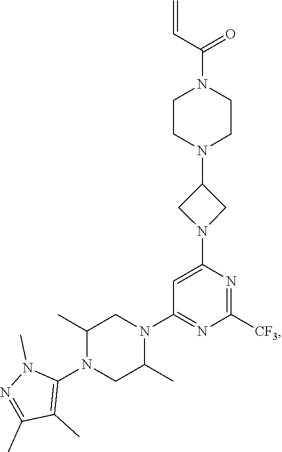 [SMILES: C=CC(=O)N1CCN(C2CN(c3cc(N4CC(C)N(c5c(C)c(C)nn5C)CC4C)nc(C(F)(F)F)n3)C2)CC1]

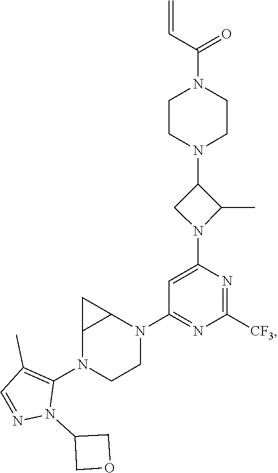 [SMILES: C=CC(=O)N1CCN(C2CN(c3cc(N4CCN(c5c(C)cnn5C5COC5)C5CC54)nc(C(F)(F)F)n3)C2C)CC1]

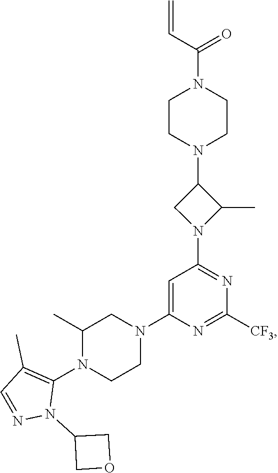 [SMILES: C=CC(=O)N1CCN(C2CN(c3cc(N4CCN(c5c(C)cnn5C5COC5)C(C)C4)nc(C(F)(F)F)n3)C2C)CC1]

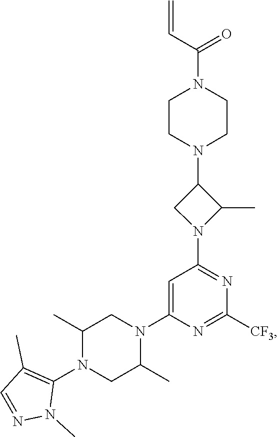 [SMILES: C=CC(=O)N1CCN(C2CN(c3cc(N4CC(C)N(c5c(C)cnn5C)CC4C)nc(C(F)(F)F)n3)C2C)CC1]

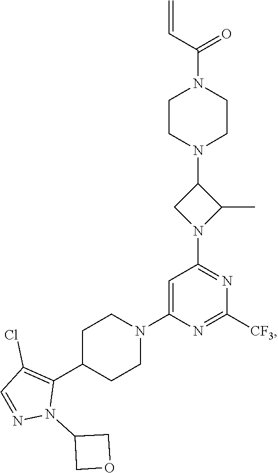 [SMILES: C=CC(=O)N1CCN(C2CN(c3cc(N4CCC(c5c(Cl)cnn5C5COC5)CC4)nc(C(F)(F)F)n3)C2C)CC1]

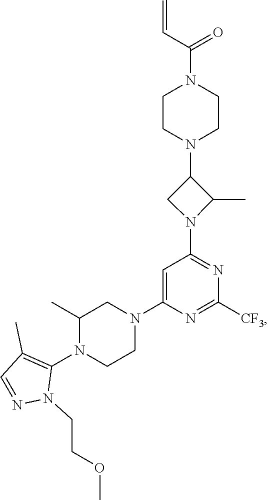 [SMILES: C=CC(=O)N1CCN(C2CN(c3cc(N4CCN(c5c(C)cnn5CCOC)C(C)C4)nc(C(F)(F)F)n3)C2C)CC1]

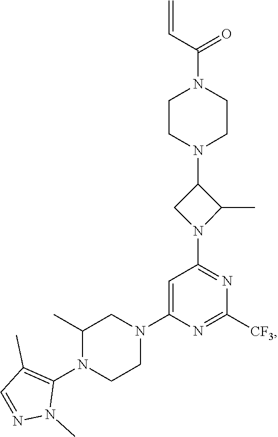 [SMILES: C=CC(=O)N1CCN(C2CN(c3cc(N4CCN(c5c(C)cnn5C)C(C)C4)nc(C(F)(F)F)n3)C2C)CC1]

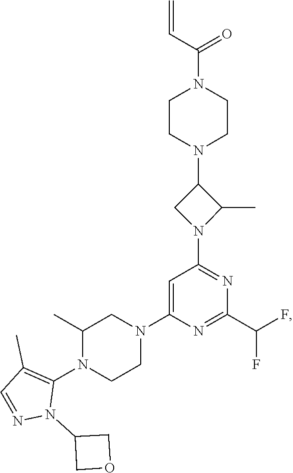 [SMILES: C=CC(=O)N1CCN(C2CN(c3cc(N4CCN(c5c(C)cnn5C5COC5)C(C)C4)nc(C(F)F)n3)C2C)CC1]

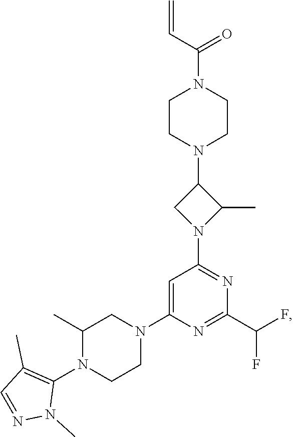 [SMILES: C=CC(=O)N1CCN(C2CN(c3cc(N4CCN(c5c(C)cnn5C)C(C)C4)nc(C(F)F)n3)C2C)CC1]

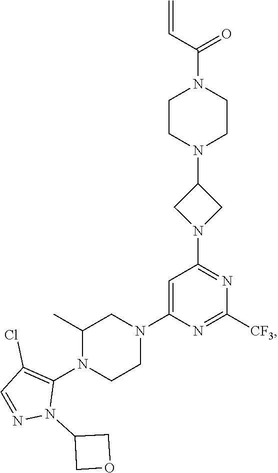 [SMILES: C=CC(=O)N1CCN(C2CN(c3cc(N4CCN(c5c(Cl)cnn5C5COC5)C(C)C4)nc(C(F)(F)F)n3)C2)CC1]

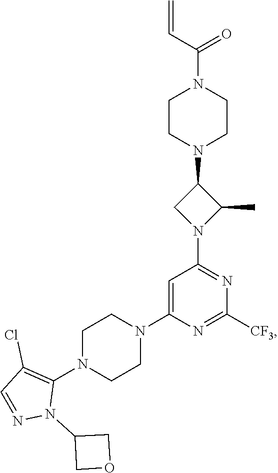 [SMILES: C=CC(=O)N1CCN([C@@H]2CN(c3cc(N4CCN(c5c(Cl)cnn5C5COC5)CC4)nc(C(F)(F)F)n3)[C@@H]2C)CC1]

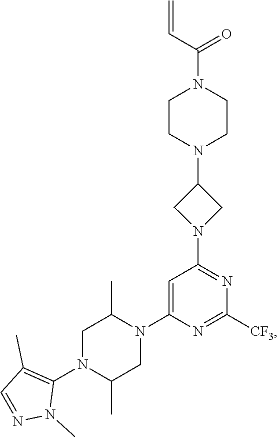 [SMILES: C=CC(=O)N1CCN(C2CN(c3cc(N4CC(C)N(c5c(C)cnn5C)CC4C)nc(C(F)(F)F)n3)C2)CC1]

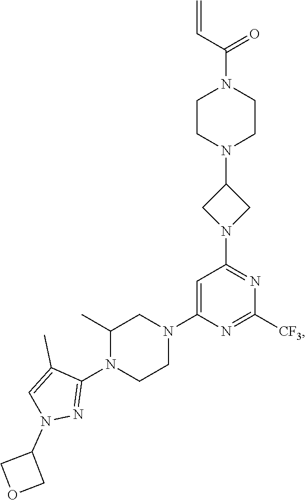 [SMILES: C=CC(=O)N1CCN(C2CN(c3cc(N4CCN(c5nn(C6COC6)cc5C)C(C)C4)nc(C(F)(F)F)n3)C2)CC1]

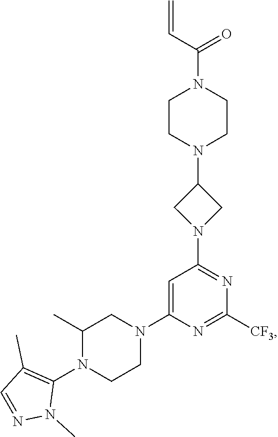 [SMILES: C=CC(=O)N1CCN(C2CN(c3cc(N4CCN(c5c(C)cnn5C)C(C)C4)nc(C(F)(F)F)n3)C2)CC1]

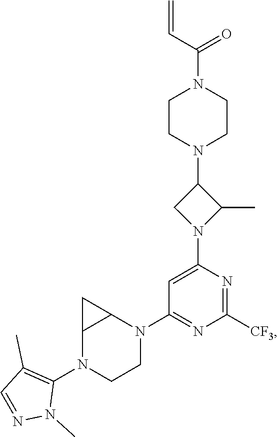 [SMILES: C=CC(=O)N1CCN(C2CN(c3cc(N4CCN(c5c(C)cnn5C)C5CC54)nc(C(F)(F)F)n3)C2C)CC1]

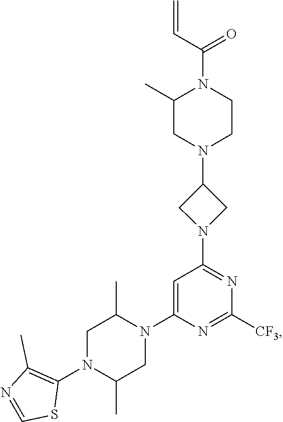 [SMILES: C=CC(=O)N1CCN(C2CN(c3cc(N4CC(C)N(c5scnc5C)CC4C)nc(C(F)(F)F)n3)C2)CC1C]

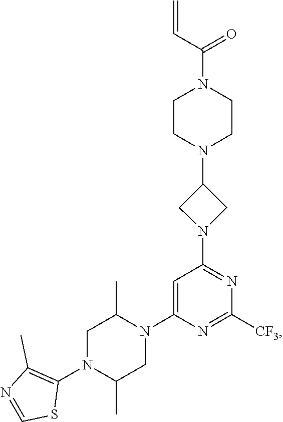 [SMILES: C=CC(=O)N1CCN(C2CN(c3cc(N4CC(C)N(c5scnc5C)CC4C)nc(C(F)(F)F)n3)C2)CC1]

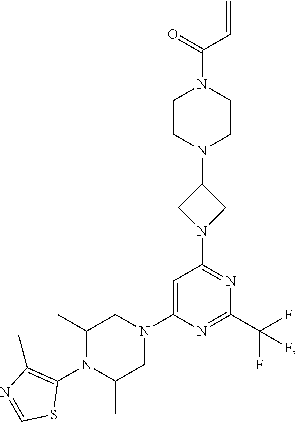 [SMILES: C=CC(=O)N1CCN(C2CN(c3cc(N4CC(C)N(c5scnc5C)C(C)C4)nc(C(F)(F)F)n3)C2)CC1]

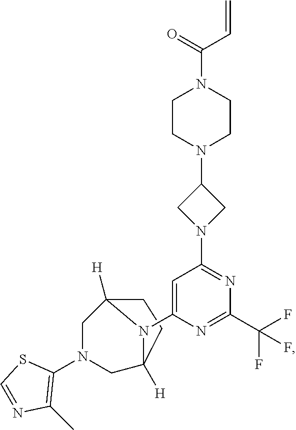 [SMILES: C=CC(=O)N1CCN(C2CN(c3cc(N4C5CCC4CN(c4scnc4C)C5)nc(C(F)(F)F)n3)C2)CC1]

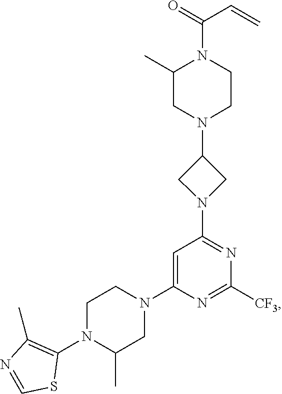 [SMILES: C=CC(=O)N1CCN(C2CN(c3cc(N4CCN(c5scnc5C)C(C)C4)nc(C(F)(F)F)n3)C2)CC1C]

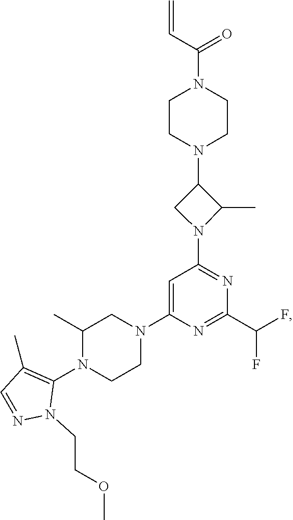 [SMILES: C=CC(=O)N1CCN(C2CN(c3cc(N4CCN(c5c(C)cnn5CCOC)C(C)C4)nc(C(F)F)n3)C2C)CC1]

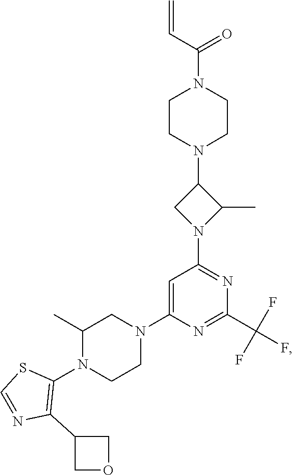 [SMILES: C=CC(=O)N1CCN(C2CN(c3cc(N4CCN(c5scnc5C5COC5)C(C)C4)nc(C(F)(F)F)n3)C2C)CC1]

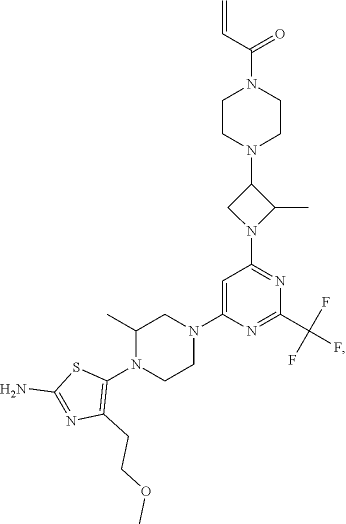 [SMILES: C=CC(=O)N1CCN(C2CN(c3cc(N4CCN(c5sc(N)nc5CCOC)C(C)C4)nc(C(F)(F)F)n3)C2C)CC1]

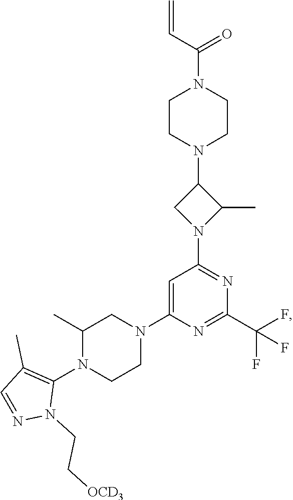

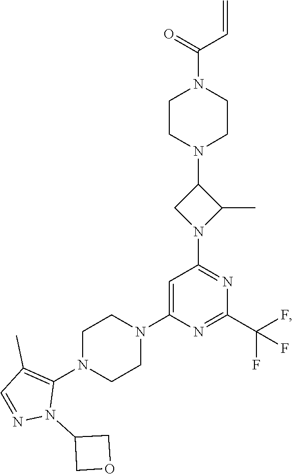 [SMILES: C=CC(=O)N1CCN(C2CN(c3cc(N4CCN(c5c(C)cnn5C5COC5)CC4)nc(C(F)(F)F)n3)C2C)CC1]

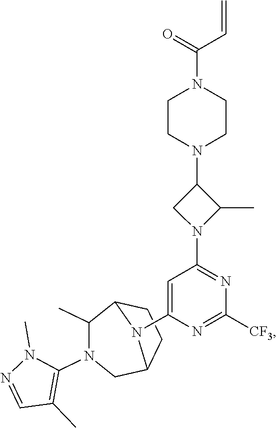 [SMILES: C=CC(=O)N1CCN(C2CN(c3cc(N4C5CCC4C(C)N(c4c(C)cnn4C)C5)nc(C(F)(F)F)n3)C2C)CC1]

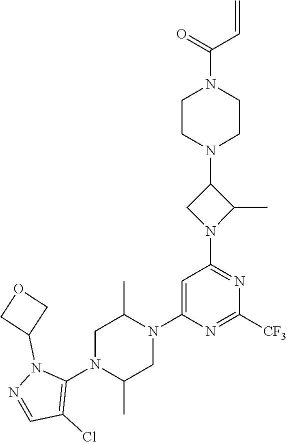 [SMILES: C=CC(=O)N1CCN(C2CN(c3cc(N4CC(C)N(c5c(Cl)cnn5C5COC5)CC4C)nc(C(F)(F)F)n3)C2C)CC1]

In some cases, the compound of Formula (I) is selected from the list of compounds in Table D.

In some cases, A is N and X is,

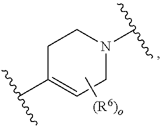

and the disclosure provides compounds of Formula (IE):

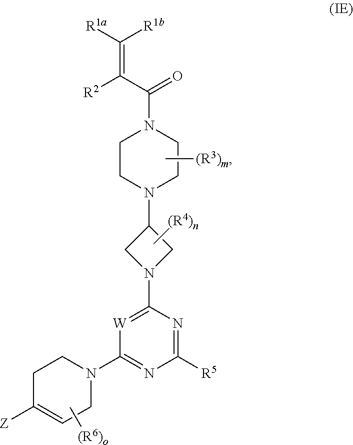

and pharmaceutically acceptable salts thereof, wherein the substituents are as previously defined herein, Contemplated compounds of Formula (IE) include, but are not limited to:

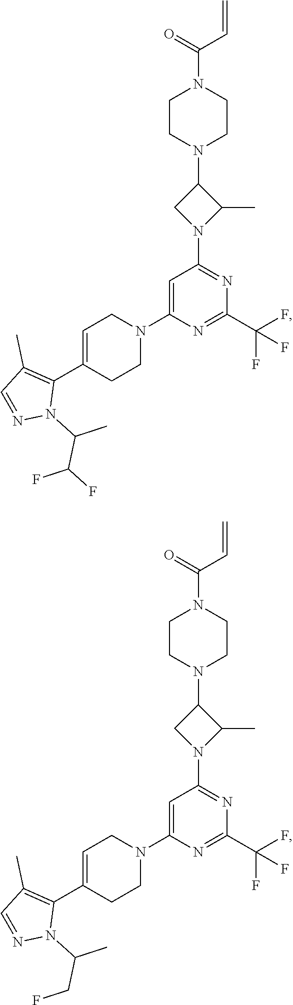 [SMILES: C=CC(=O)N1CCN(C2CN(c3cc(N4CC=C(c5c(C)cnn5C(C)C(F)F)CC4)nc(C(F)(F)F)n3)C2C)CC1 ; C=CC(=O)N1CCN(C2CN(c3cc(N4CC=C(c5c(C)cnn5C(C)CF)CC4)nc(C(F)(F)F)n3)C2C)CC1]

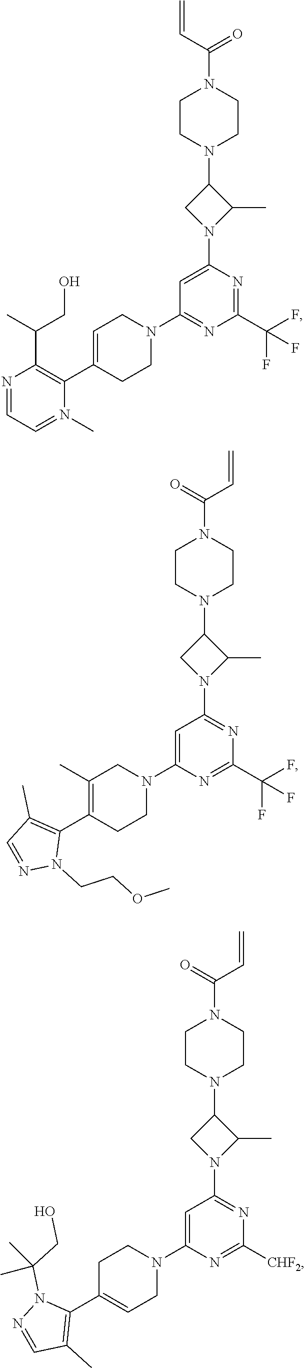

and pharmaceutically acceptable salts thereof.

In some cases, A is N and X is

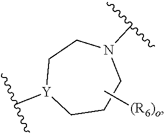

and the disclosure provides compounds of Formula (IF):

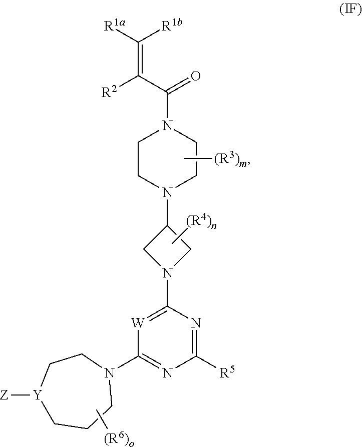

and pharmaceutically acceptable salts thereof, wherein the substituents are as previously defined herein. A contemplated compound of Formula (IF) is:

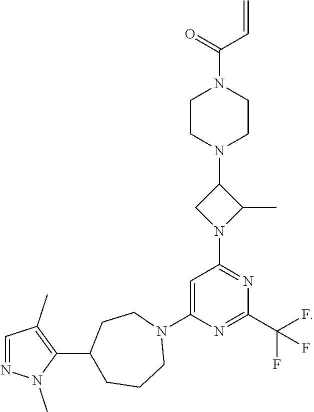 [SMILES: C=CC(=O)N1CCN(C2CN(c3cc(N4CCCC(c5c(C)cnn5C)CC4)nc(C(F)(F)F)n3)C2C)CC1]

In some cases, the disclosure provides compounds of Formula (I) wherein A is N; X is

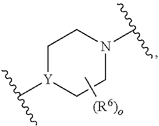

and Z is optionally substituted pyrazolyl. Examples of such compounds include, but are not limited to:

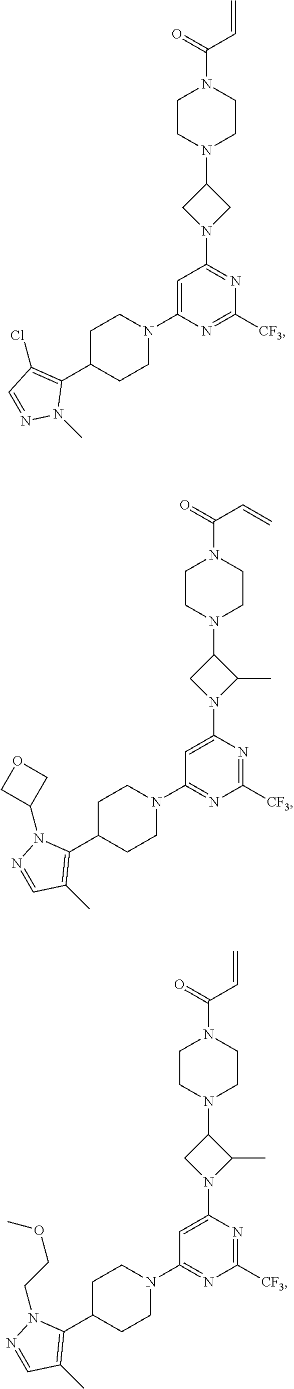 [SMILES: C=CC(=O)N1CCN(C2CN(c3cc(N4CCC(c5c(Cl)cnn5C)CC4)nc(C(F)(F)F)n3)C2)CC1 ; C=CC(=O)N1CCN(C2CN(c3cc(N4CCC(c5c(C)cnn5C5COC5)CC4)nc(C(F)(F)F)n3)C2C)CC1 ; C=CC(=O)N1CCN(C2CN(c3cc(N4CCC(c5c(C)cnn5CCOC)CC4)nc(C(F)(F)F)n3)C2C)CC1]

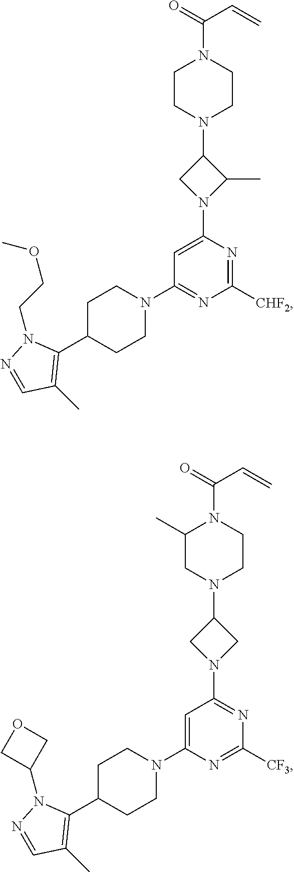 [SMILES: C=CC(=O)N1CCN(C2CN(c3cc(N4CCC(c5c(C)cnn5CCOC)CC4)nc(C(F)F)n3)C2C)CC1 ; C=CC(=O)N1CCN(C2CN(c3cc(N4CCC(c5c(C)cnn5C5COC5)CC4)nc(C(F)(F)F)n3)C2)CC1C]

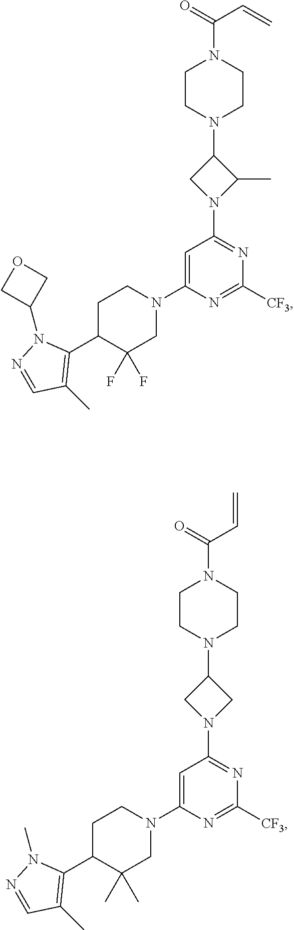 [SMILES: C=CC(=O)N1CCN(C2CN(c3cc(N4CCC(c5c(C)cnn5C5COC5)C(F)(F)C4)nc(C(F)(F)F)n3)C2C)CC1 ; C=CC(=O)N1CCN(C2CN(c3cc(N4CCC(c5c(C)cnn5C)C(C)(C)C4)nc(C(F)(F)F)n3)C2)CC1]

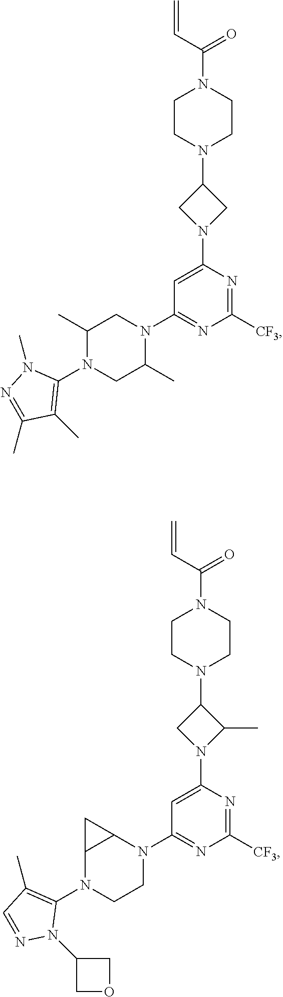 [SMILES: C=CC(=O)N1CCN(C2CN(c3cc(N4CC(C)N(c5c(C)c(C)nn5C)CC4C)nc(C(F)(F)F)n3)C2)CC1 ; C=CC(=O)N1CCN(C2CN(c3cc(N4CCN(c5c(C)cnn5C5COC5)C5CC54)nc(C(F)(F)F)n3)C2C)CC1]

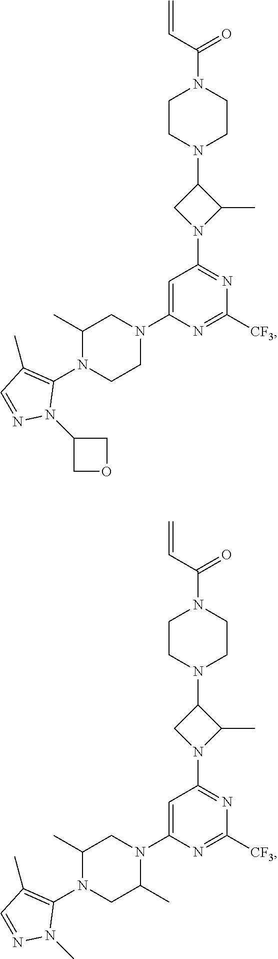 [SMILES: C=CC(=O)N1CCN(C2CN(c3cc(N4CCN(c5c(C)cnn5C5COC5)C(C)C4)nc(C(F)(F)F)n3)C2C)CC1 ; C=CC(=O)N1CCN(C2CN(c3cc(N4CC(C)N(c5c(C)cnn5C)CC4C)nc(C(F)(F)F)n3)C2C)CC1]

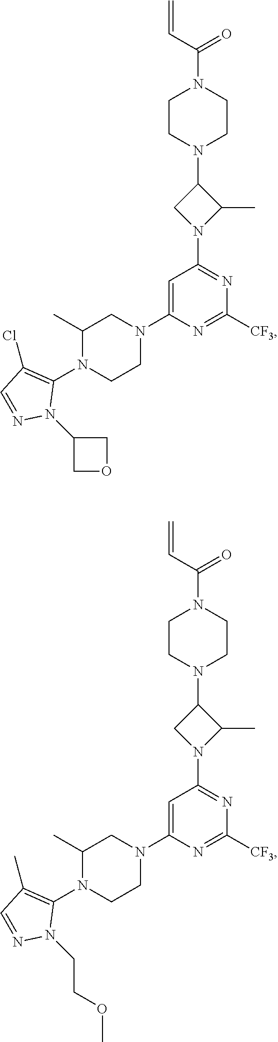 [SMILES: C=CC(=O)N1CCN(C2CN(c3cc(N4CCN(c5c(Cl)cnn5C5COC5)C(C)C4)nc(C(F)(F)F)n3)C2C)CC1 ; C=CC(=O)N1CCN(C2CN(c3cc(N4CCN(c5c(C)cnn5CCOC)C(C)C4)nc(C(F)(F)F)n3)C2C)CC1]

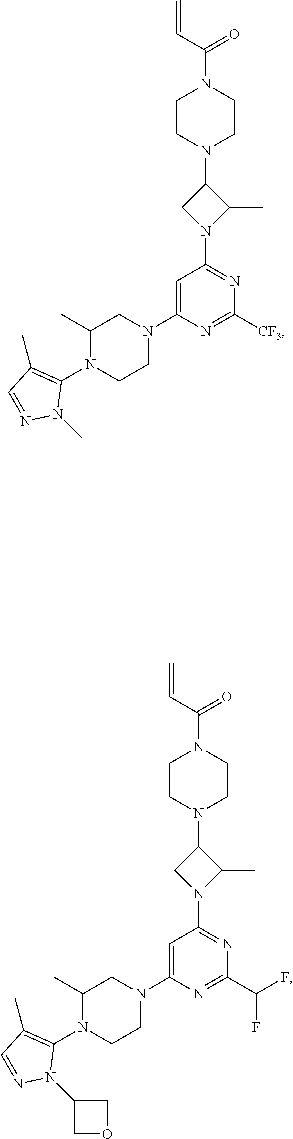 [SMILES: C=CC(=O)N1CCN(C2CN(c3cc(N4CCN(c5c(C)cnn5C)C(C)C4)nc(C(F)(F)F)n3)C2C)CC1 ; C=CC(=O)N1CCN(C2CN(c3cc(N4CCN(c5c(C)cnn5C5COC5)C(C)C4)nc(C(F)F)n3)C2C)CC1]

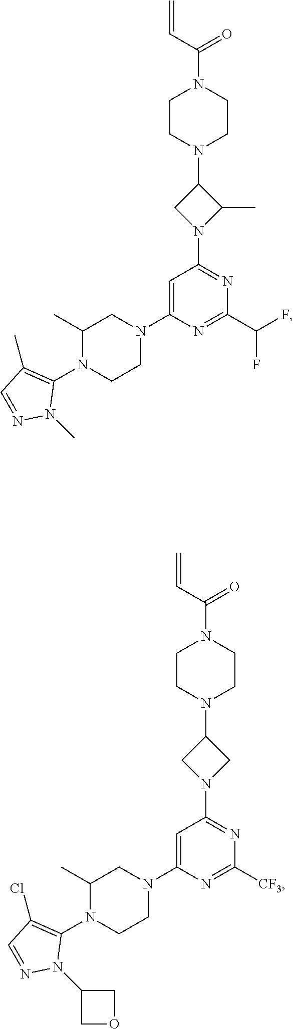 [SMILES: C=CC(=O)N1CCN(C2CN(c3cc(N4CCN(c5c(C)cnn5C)C(C)C4)nc(C(F)F)n3)C2C)CC1 ; C=CC(=O)N1CCN(C2CN(c3cc(N4CCN(c5c(Cl)cnn5C5COC5)C(C)C4)nc(C(F)(F)F)n3)C2)CC1]

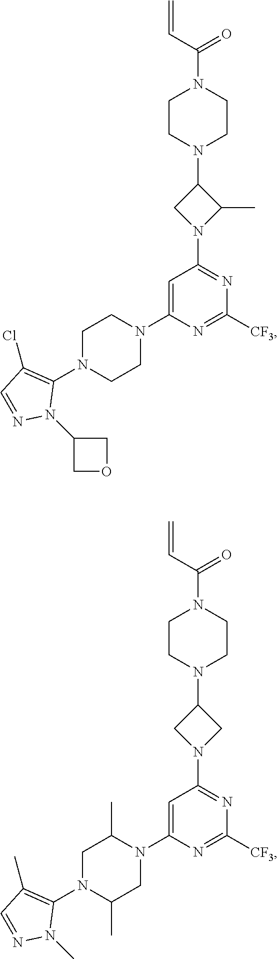 [SMILES: C=CC(=O)N1CCN(C2CN(c3cc(N4CCN(c5c(Cl)cnn5C5COC5)CC4)nc(C(F)(F)F)n3)C2C)CC1 ; C=CC(=O)N1CCN(C2CN(c3cc(N4CC(C)N(c5c(C)cnn5C)CC4C)nc(C(F)(F)F)n3)C2)CC1]

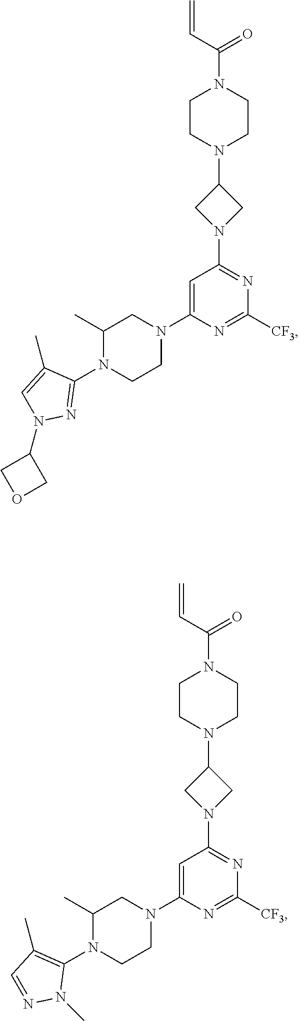 [SMILES: C=CC(=O)N1CCN(C2CN(c3cc(N4CCN(c5nn(C6COC6)cc5C)C(C)C4)nc(C(F)(F)F)n3)C2)CC1 ; C=CC(=O)N1CCN(C2CN(c3cc(N4CCN(c5c(C)cnn5C)C(C)C4)nc(C(F)(F)F)n3)C2)CC1]

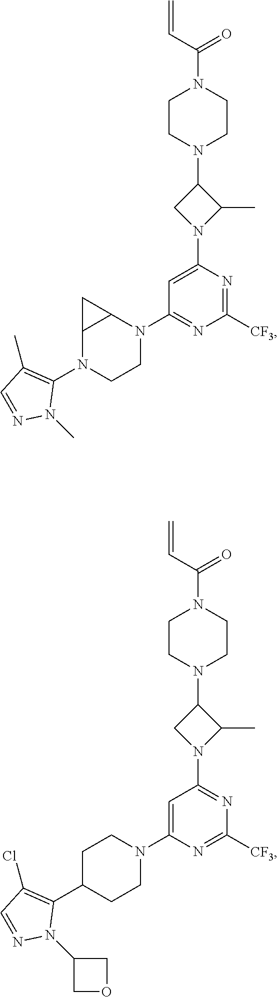 [SMILES: C=CC(=O)N1CCN(C2CN(c3cc(N4CCN(c5c(C)cnn5C)C5CC54)nc(C(F)(F)F)n3)C2C)CC1 ; C=CC(=O)N1CCN(C2CN(c3cc(N4CCC(c5c(Cl)cnn5C5COC5)CC4)nc(C(F)(F)F)n3)C2C)CC1]

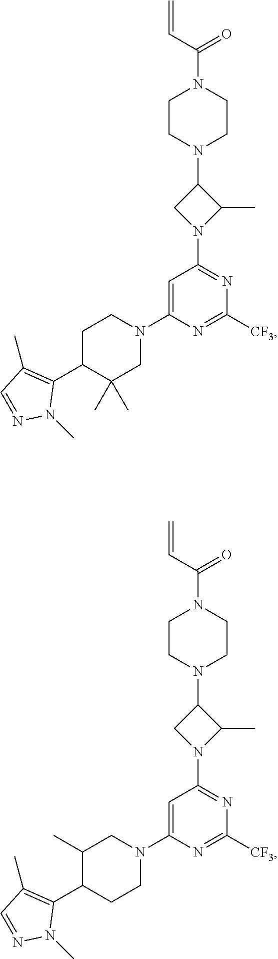 [SMILES: C=CC(=O)N1CCN(C2CN(c3cc(N4CCC(c5c(C)cnn5C)C(C)(C)C4)nc(C(F)(F)F)n3)C2C)CC1 ; C=CC(=O)N1CCN(C2CN(c3cc(N4CCC(c5c(C)cnn5C)C(C)C4)nc(C(F)(F)F)n3)C2C)CC1]

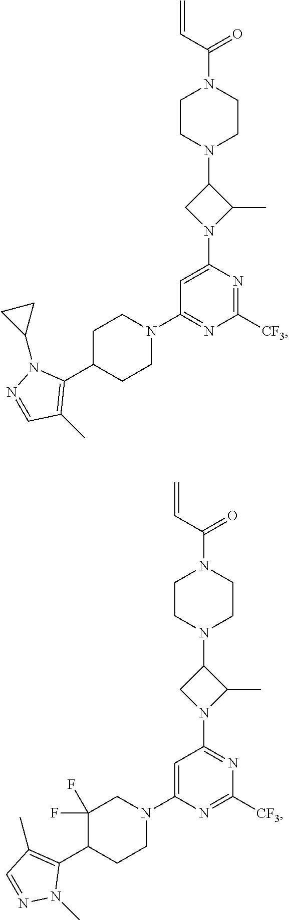 [SMILES: C=CC(=O)N1CCN(C2CN(c3cc(N4CCC(c5c(C)cnn5C5CC5)CC4)nc(C(F)(F)F)n3)C2C)CC1 ; C=CC(=O)N1CCN(C2CN(c3cc(N4CCC(c5c(C)cnn5C)C(F)(F)C4)nc(C(F)(F)F)n3)C2C)CC1]

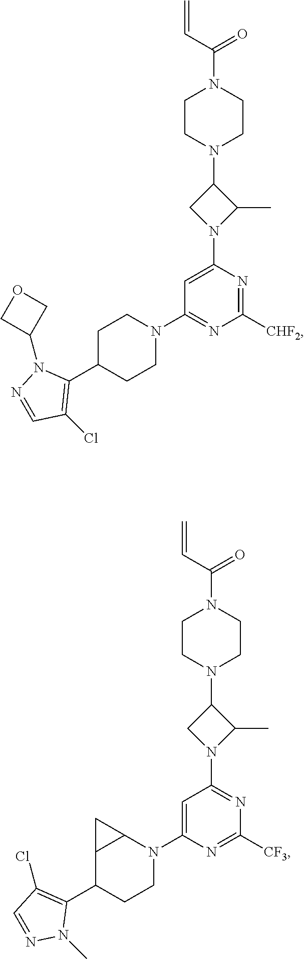 [SMILES: C=CC(=O)N1CCN(C2CN(c3cc(N4CCC(c5c(Cl)cnn5C5COC5)CC4)nc(C(F)F)n3)C2C)CC1 ; C=CC(=O)N1CCN(C2CN(c3cc(N4CCC(c5c(Cl)cnn5C)C5CC54)nc(C(F)(F)F)n3)C2C)CC1]

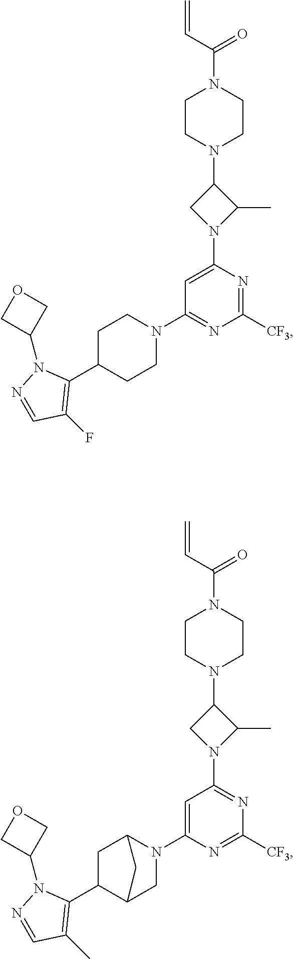 [SMILES: C=CC(=O)N1CCN(C2CN(c3cc(N4CCC(c5c(F)cnn5C5COC5)CC4)nc(C(F)(F)F)n3)C2C)CC1 ; C=CC(=O)N1CCN(C2CN(c3cc(N4CC5CC4CC5c4c(C)cnn4C4COC4)nc(C(F)(F)F)n3)C2C)CC1]

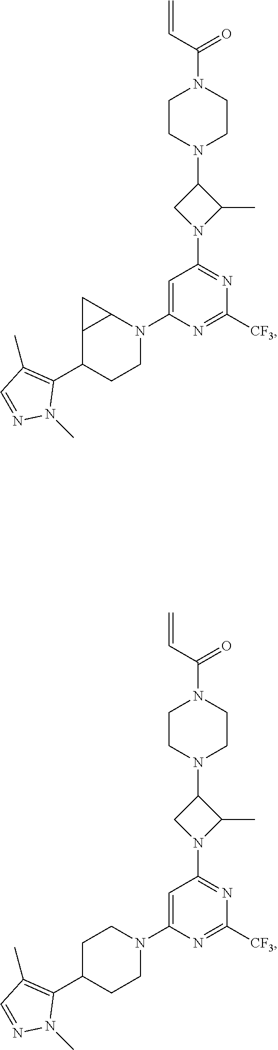 [SMILES: C=CC(=O)N1CCN(C2CN(c3cc(N4CCC(c5c(C)cnn5C)C5CC54)nc(C(F)(F)F)n3)C2C)CC1 ; C=CC(=O)N1CCN(C2CN(c3cc(N4CCC(c5c(C)cnn5C)CC4)nc(C(F)(F)F)n3)C2C)CC1]

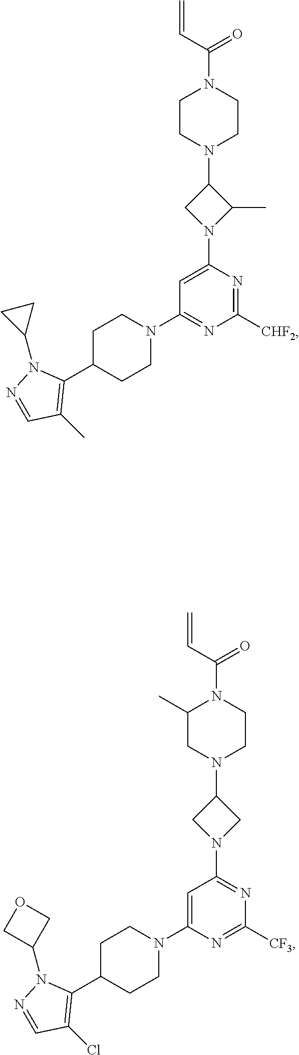 [SMILES: C=CC(=O)N1CCN(C2CN(c3cc(N4CCC(c5c(C)cnn5C5CC5)CC4)nc(C(F)F)n3)C2C)CC1 ; C=CC(=O)N1CCN(C2CN(c3cc(N4CCC(c5c(Cl)cnn5C5COC5)CC4)nc(C(F)(F)F)n3)C2)CC1C]

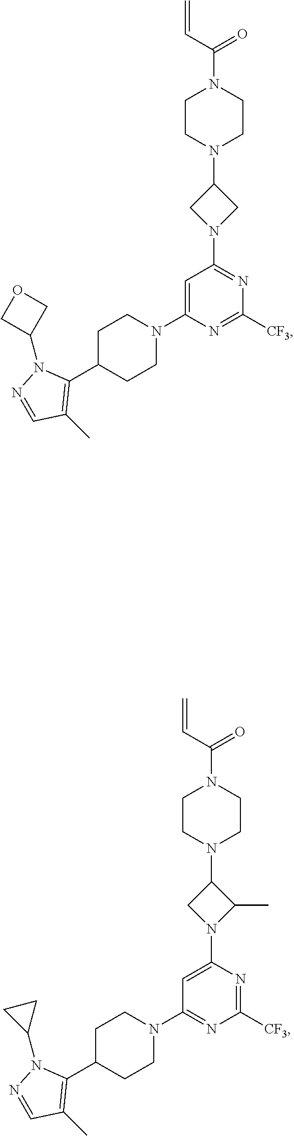 [SMILES: C=CC(=O)N1CCN(C2CN(c3cc(N4CCC(c5c(C)cnn5C5COC5)CC4)nc(C(F)(F)F)n3)C2)CC1 ; C=CC(=O)N1CCN(C2CN(c3cc(N4CCC(c5c(C)cnn5C5CC5)CC4)nc(C(F)(F)F)n3)C2C)CC1]

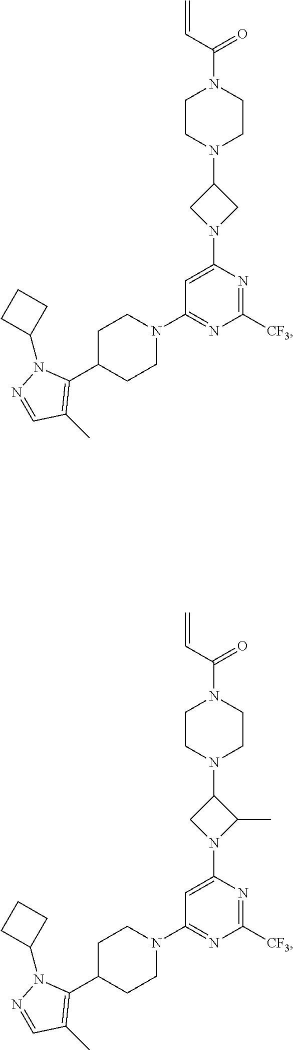 [SMILES: C=CC(=O)N1CCN(C2CN(c3cc(N4CCC(c5c(C)cnn5C5CCC5)CC4)nc(C(F)(F)F)n3)C2)CC1 ; C=CC(=O)N1CCN(C2CN(c3cc(N4CCC(c5c(C)cnn5C5CCC5)CC4)nc(C(F)(F)F)n3)C2C)CC1]

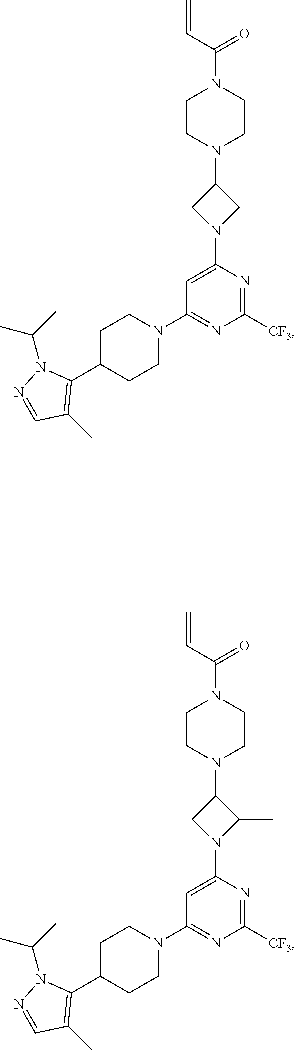 [SMILES: C=CC(=O)N1CCN(C2CN(c3cc(N4CCC(c5c(C)cnn5C(C)C)CC4)nc(C(F)(F)F)n3)C2)CC1 ; C=CC(=O)N1CCN(C2CN(c3cc(N4CCC(c5c(C)cnn5C(C)C)CC4)nc(C(F)(F)F)n3)C2C)CC1]

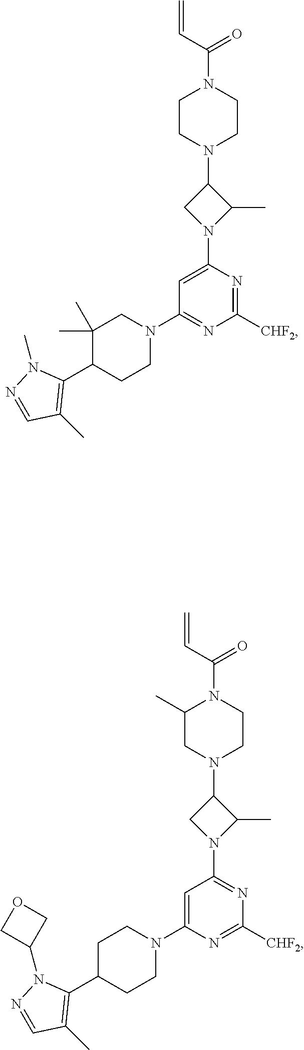 [SMILES: C=CC(=O)N1CCN(C2CN(c3cc(N4CCC(c5c(C)cnn5C)C(C)(C)C4)nc(C(F)F)n3)C2C)CC1 ; C=CC(=O)N1CCN(C2CN(c3cc(N4CCC(c5c(C)cnn5C5COC5)CC4)nc(C(F)F)n3)C2C)CC1C]

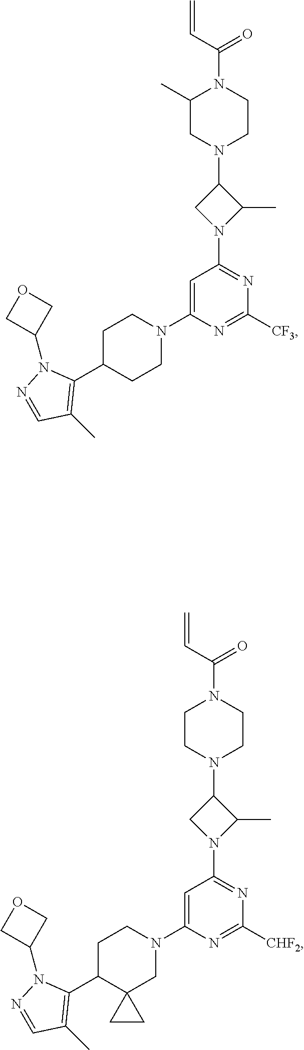 [SMILES: C=CC(=O)N1CCN(C2CN(c3cc(N4CCC(c5c(C)cnn5C5COC5)CC4)nc(C(F)(F)F)n3)C2C)CC1C ; C=CC(=O)N1CCN(C2CN(c3cc(N4CCC(c5c(C)cnn5C5COC5)C5(CC5)C4)nc(C(F)F)n3)C2C)CC1]

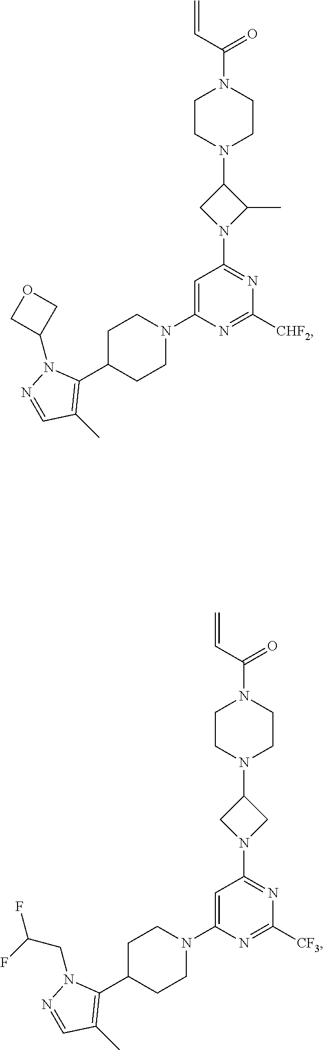 [SMILES: C=CC(=O)N1CCN(C2CN(c3cc(N4CCC(c5c(C)cnn5C5COC5)CC4)nc(C(F)F)n3)C2C)CC1 ; C=CC(=O)N1CCN(C2CN(c3cc(N4CCC(c5c(C)cnn5CC(F)F)CC4)nc(C(F)(F)F)n3)C2)CC1]

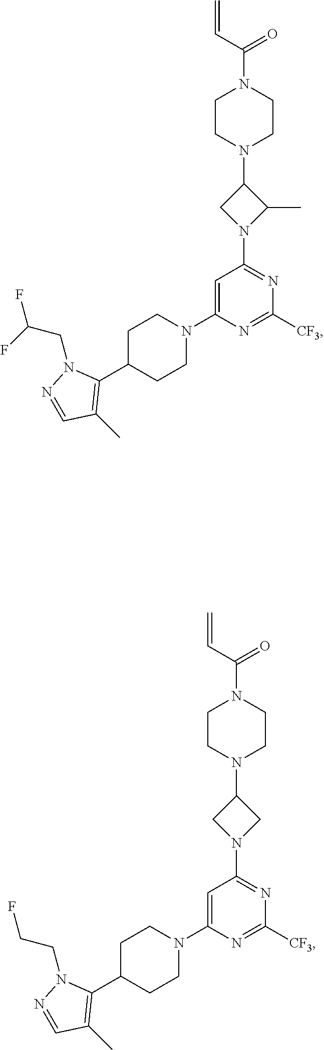 [SMILES: C=CC(=O)N1CCN(C2CN(c3cc(N4CCC(c5c(C)cnn5CC(F)F)CC4)nc(C(F)(F)F)n3)C2C)CC1 ; C=CC(=O)N1CCN(C2CN(c3cc(N4CCC(c5c(C)cnn5CCF)CC4)nc(C(F)(F)F)n3)C2)CC1]

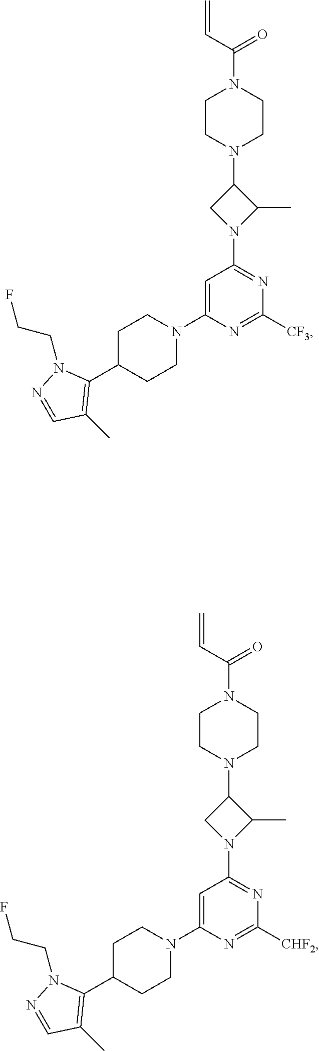 [SMILES: C=CC(=O)N1CCN(C2CN(c3cc(N4CCC(c5c(C)cnn5CCF)CC4)nc(C(F)(F)F)n3)C2C)CC1 ; C=CC(=O)N1CCN(C2CN(c3cc(N4CCC(c5c(C)cnn5CCF)CC4)nc(C(F)F)n3)C2C)CC1]

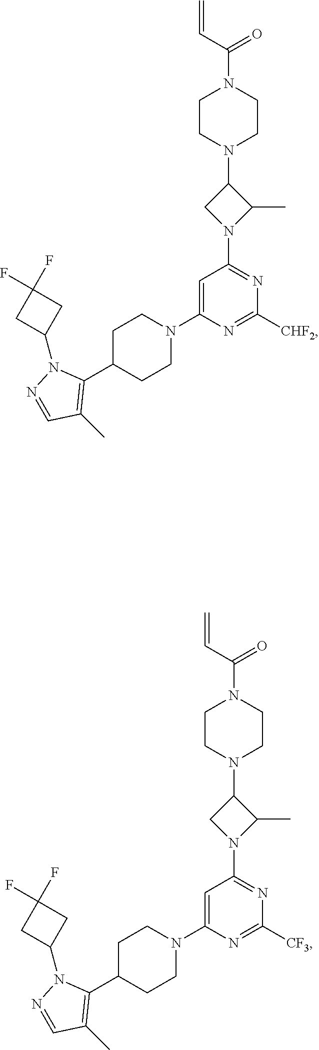 [SMILES: C=CC(=O)N1CCN(C2CN(c3cc(N4CCC(c5c(C)cnn5C5CC(F)(F)C5)CC4)nc(C(F)F)n3)C2C)CC1 ; C=CC(=O)N1CCN(C2CN(c3cc(N4CCC(c5c(C)cnn5C5CC(F)(F)C5)CC4)nc(C(F)(F)F)n3)C2C)CC1]

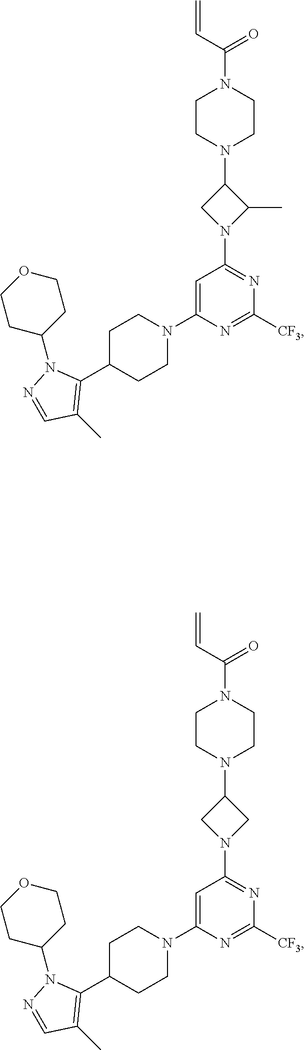 [SMILES: C=CC(=O)N1CCN(C2CN(c3cc(N4CCC(c5c(C)cnn5C5CCOCC5)CC4)nc(C(F)(F)F)n3)C2C)CC1 ; C=CC(=O)N1CCN(C2CN(c3cc(N4CCC(c5c(C)cnn5C5CCOCC5)CC4)nc(C(F)(F)F)n3)C2)CC1]

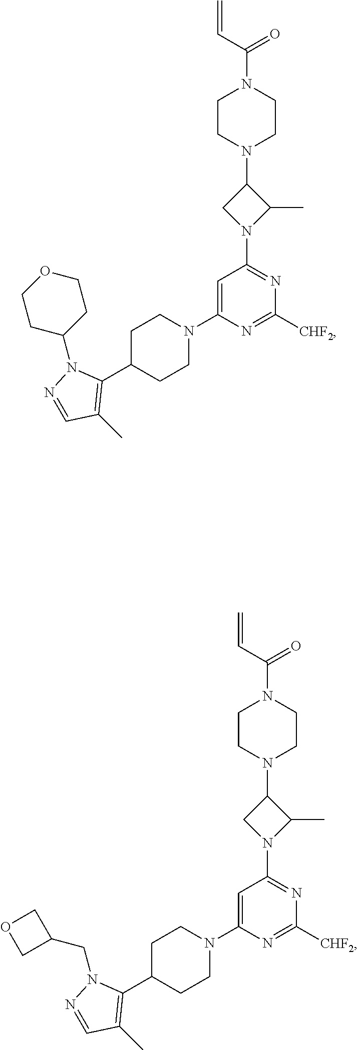 [SMILES: C=CC(=O)N1CCN(C2CN(c3cc(N4CCC(c5c(C)cnn5C5CCOCC5)CC4)nc(C(F)F)n3)C2C)CC1 ; C=CC(=O)N1CCN(C2CN(c3cc(N4CCC(c5c(C)cnn5CC5COC5)CC4)nc(C(F)F)n3)C2C)CC1]

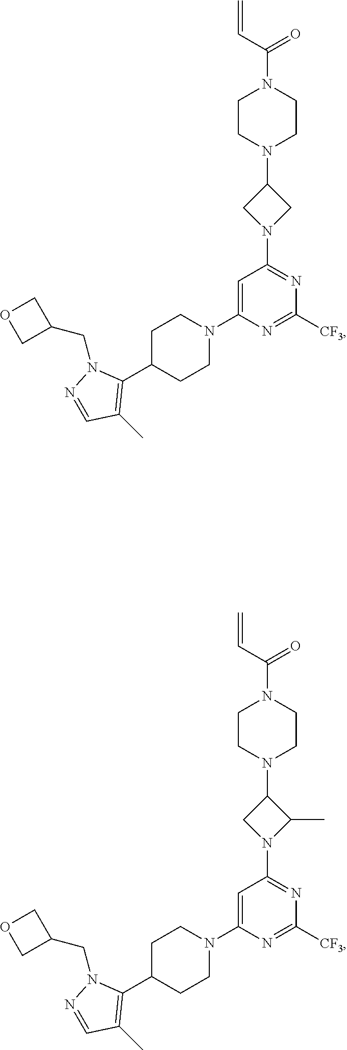 [SMILES: C=CC(=O)N1CCN(C2CN(c3cc(N4CCC(c5c(C)cnn5CC5COC5)CC4)nc(C(F)(F)F)n3)C2)CC1 ; C=CC(=O)N1CCN(C2CN(c3cc(N4CCC(c5c(C)cnn5CC5COC5)CC4)nc(C(F)(F)F)n3)C2C)CC1]

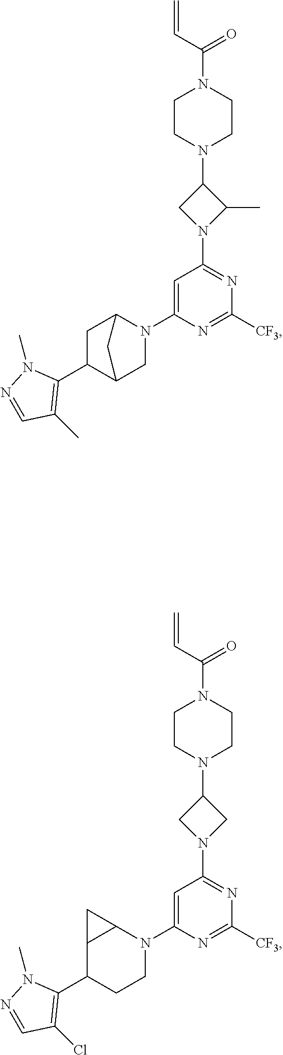 [SMILES: C=CC(=O)N1CCN(C2CN(c3cc(N4CC5CC4CC5c4c(C)cnn4C)nc(C(F)(F)F)n3)C2C)CC1 ; C=CC(=O)N1CCN(C2CN(c3cc(N4CCC(c5c(Cl)cnn5C)C5CC54)nc(C(F)(F)F)n3)C2)CC1]

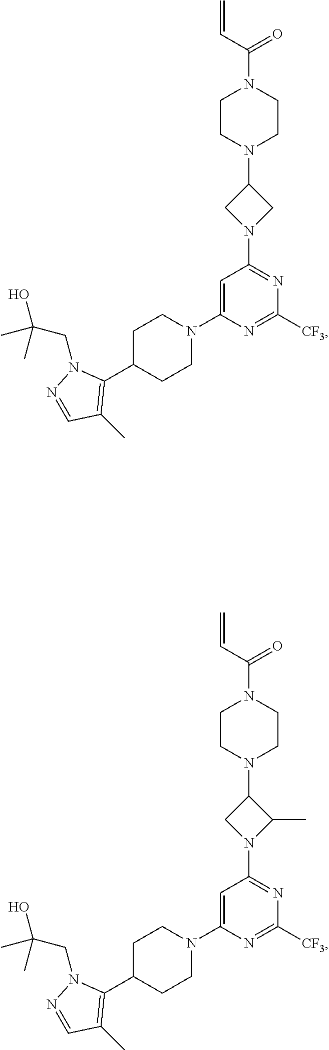 [SMILES: C=CC(=O)N1CCN(C2CN(c3cc(N4CCC(c5c(C)cnn5CC(C)(C)O)CC4)nc(C(F)(F)F)n3)C2)CC1 ; C=CC(=O)N1CCN(C2CN(c3cc(N4CCC(c5c(C)cnn5CC(C)(C)O)CC4)nc(C(F)(F)F)n3)C2C)CC1]

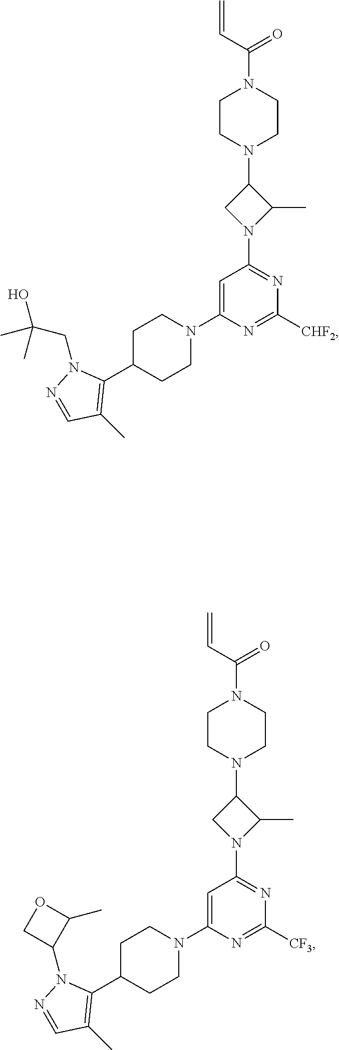 [SMILES: C=CC(=O)N1CCN(C2CN(c3cc(N4CCC(c5c(C)cnn5CC(C)(C)O)CC4)nc(C(F)F)n3)C2C)CC1 ; C=CC(=O)N1CCN(C2CN(c3cc(N4CCC(c5c(C)cnn5C5COC5C)CC4)nc(C(F)(F)F)n3)C2C)CC1]

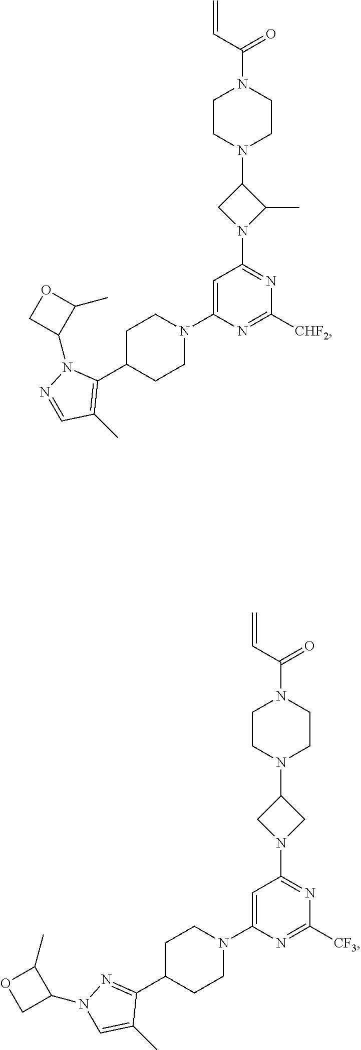 [SMILES: C=CC(=O)N1CCN(C2CN(c3cc(N4CCC(c5c(C)cnn5C5COC5C)CC4)nc(C(F)F)n3)C2C)CC1 ; C=CC(=O)N1CCN(C2CN(c3cc(N4CCC(c5nn(C6COC6C)cc5C)CC4)nc(C(F)(F)F)n3)C2)CC1]

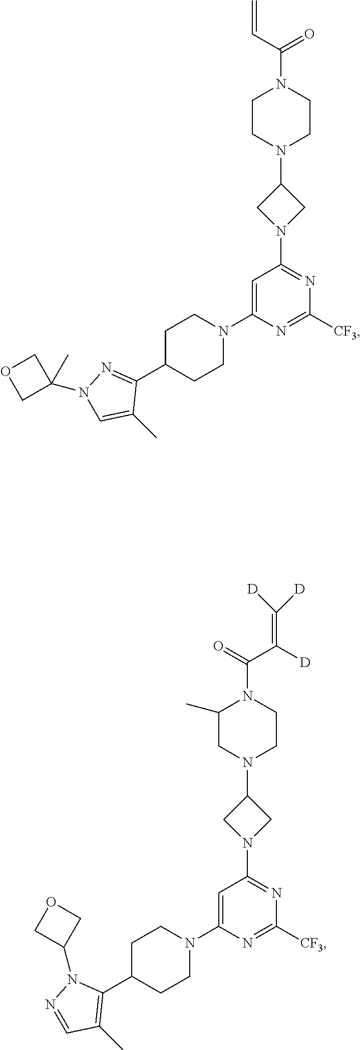 [SMILES: C=CC(=O)N1CCN(C2CN(c3cc(N4CCC(c5nn(C6(C)COC6)cc5C)CC4)nc(C(F)(F)F)n3)C2)CC1 ; [2H]C([2H])=C([2H])C(=O)N1CCN(C2CN(c3cc(N4CCC(c5c(C)cnn5C5COC5)CC4)nc(C(F)(F)F)n3)C2)CC1C]

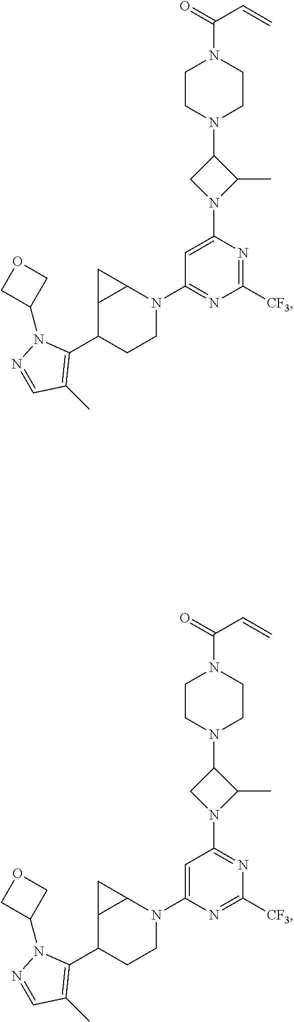 [SMILES: C=CC(=O)N1CCN(C2CN(c3cc(N4CCC(c5c(C)cnn5C5COC5)C5CC54)nc(C(F)(F)F)n3)C2C)CC1 ; C=CC(=O)N1CCN(C2CN(c3cc(N4CCC(c5c(C)cnn5C5COC5)C5CC54)nc(C(F)(F)F)n3)C2C)CC1]

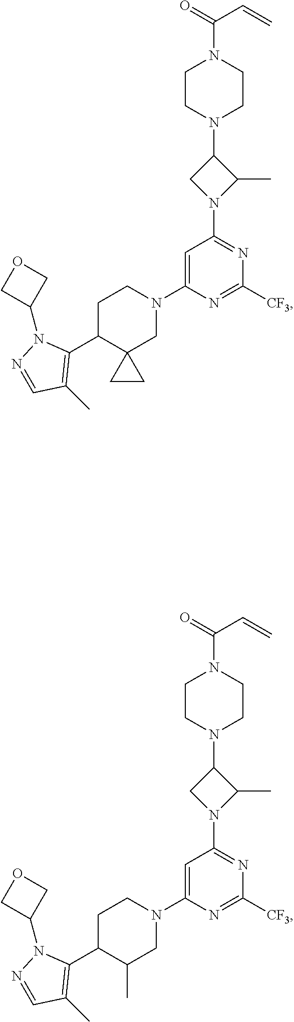 [SMILES: C=CC(=O)N1CCN(C2CN(c3cc(N4CCC(c5c(C)cnn5C5COC5)C5(CC5)C4)nc(C(F)(F)F)n3)C2C)CC1 ; C=CC(=O)N1CCN(C2CN(c3cc(N4CCC(c5c(C)cnn5C5COC5)C(C)C4)nc(C(F)(F)F)n3)C2C)CC1]

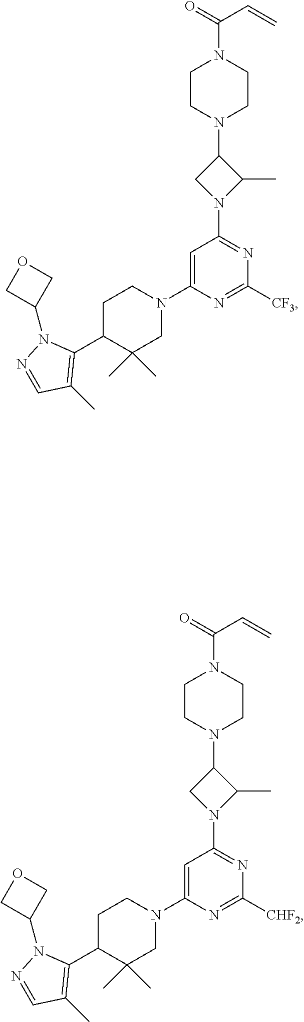 [SMILES: C=CC(=O)N1CCN(C2CN(c3cc(N4CCC(c5c(C)cnn5C5COC5)C(C)(C)C4)nc(C(F)(F)F)n3)C2C)CC1 ; C=CC(=O)N1CCN(C2CN(c3cc(N4CCC(c5c(C)cnn5C5COC5)C(C)(C)C4)nc(C(F)F)n3)C2C)CC1]

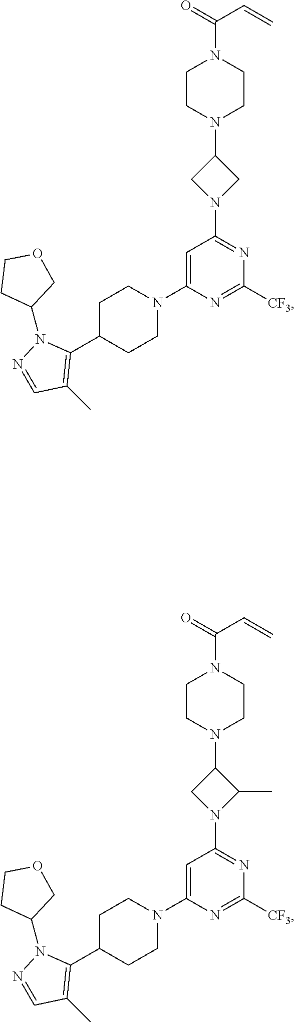 [SMILES: C=CC(=O)N1CCN(C2CN(c3cc(N4CCC(c5c(C)cnn5C5CCOC5)CC4)nc(C(F)(F)F)n3)C2)CC1 ; C=CC(=O)N1CCN(C2CN(c3cc(N4CCC(c5c(C)cnn5C5CCOC5)CC4)nc(C(F)(F)F)n3)C2C)CC1]

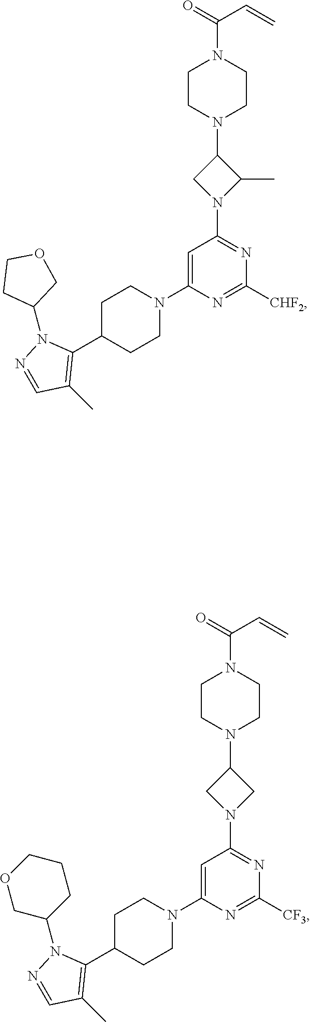 [SMILES: C=CC(=O)N1CCN(C2CN(c3cc(N4CCC(c5c(C)cnn5C5CCOC5)CC4)nc(C(F)F)n3)C2C)CC1 ; C=CC(=O)N1CCN(C2CN(c3cc(N4CCC(c5c(C)cnn5C5CCCOC5)CC4)nc(C(F)(F)F)n3)C2)CC1]

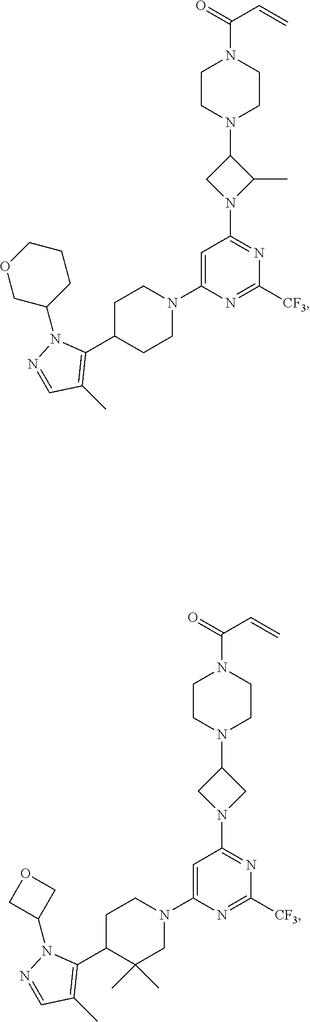 [SMILES: C=CC(=O)N1CCN(C2CN(c3cc(N4CCC(c5c(C)cnn5C5CCCOC5)CC4)nc(C(F)(F)F)n3)C2C)CC1 ; C=CC(=O)N1CCN(C2CN(c3cc(N4CCC(c5c(C)cnn5C5COC5)C(C)(C)C4)nc(C(F)(F)F)n3)C2)CC1]

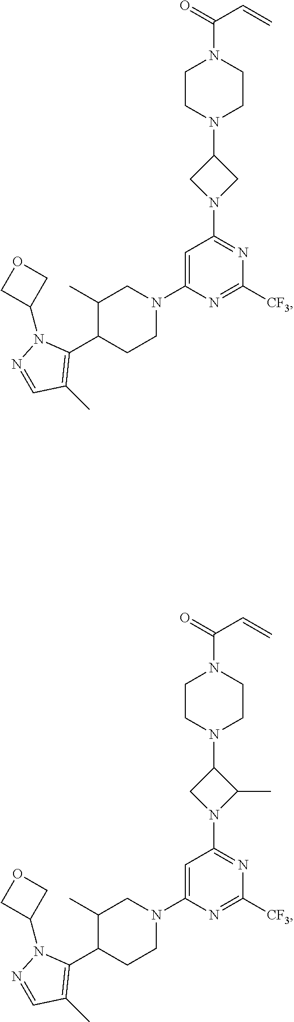 [SMILES: C=CC(=O)N1CCN(C2CN(c3cc(N4CCC(c5c(C)cnn5C5COC5)C(C)C4)nc(C(F)(F)F)n3)C2)CC1 ; C=CC(=O)N1CCN(C2CN(c3cc(N4CCC(c5c(C)cnn5C5COC5)C(C)C4)nc(C(F)(F)F)n3)C2C)CC1]

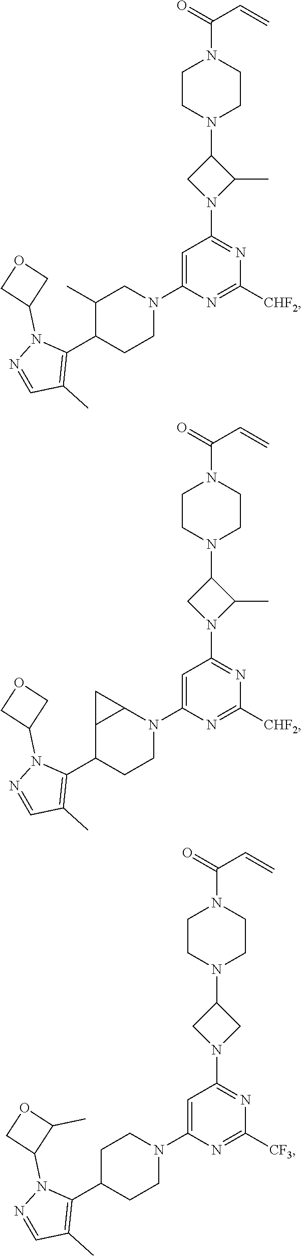 [SMILES: C=CC(=O)N1CCN(C2CN(c3cc(N4CCC(c5c(C)cnn5C5COC5)C(C)C4)nc(C(F)F)n3)C2C)CC1 ; C=CC(=O)N1CCN(C2CN(c3cc(N4CCC(c5c(C)cnn5C5COC5)C5CC54)nc(C(F)F)n3)C2C)CC1 ; C=CC(=O)N1CCN(C2CN(c3cc(N4CCC(c5c(C)cnn5C5COC5C)CC4)nc(C(F)(F)F)n3)C2)CC1]

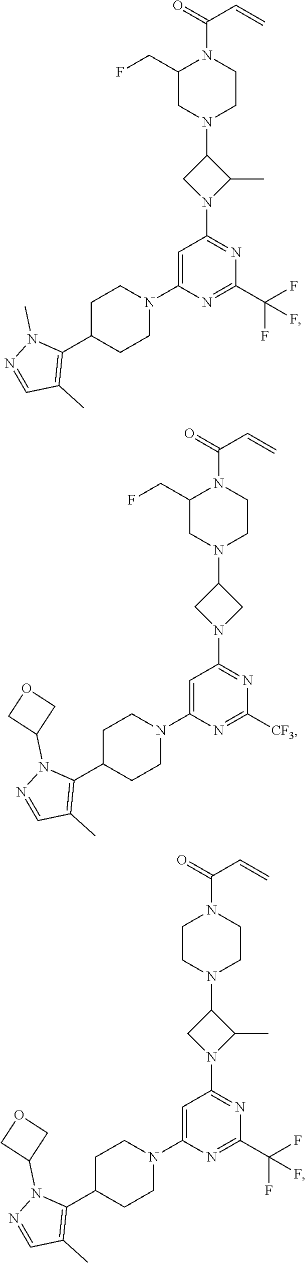 [SMILES: C=CC(=O)N1CCN(C2CN(c3cc(N4CCC(c5c(C)cnn5C)CC4)nc(C(F)(F)F)n3)C2C)CC1CF ; C=CC(=O)N1CCN(C2CN(c3cc(N4CCC(c5c(C)cnn5C5COC5)CC4)nc(C(F)(F)F)n3)C2)CC1CF ; C=CC(=O)N1CCN(C2CN(c3cc(N4CCC(c5c(C)cnn5C5COC5)CC4)nc(C(F)(F)F)n3)C2C)CC1]

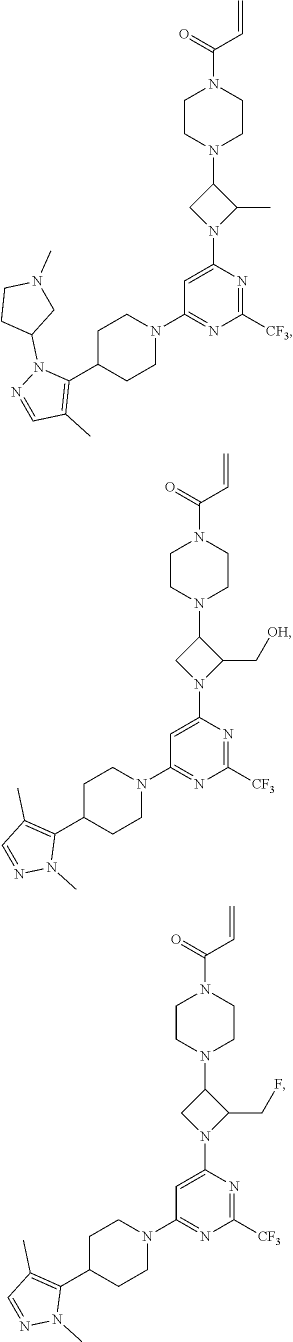 [SMILES: C=CC(=O)N1CCN(C2CN(c3cc(N4CCC(c5c(C)cnn5C5CCN(C)C5)CC4)nc(C(F)(F)F)n3)C2C)CC1 ; C=CC(=O)N1CCN(C2CN(c3cc(N4CCC(c5c(C)cnn5C)CC4)nc(C(F)(F)F)n3)C2CO)CC1 ; C=CC(=O)N1CCN(C2CN(c3cc(N4CCC(c5c(C)cnn5C)CC4)nc(C(F)(F)F)n3)C2CF)CC1]

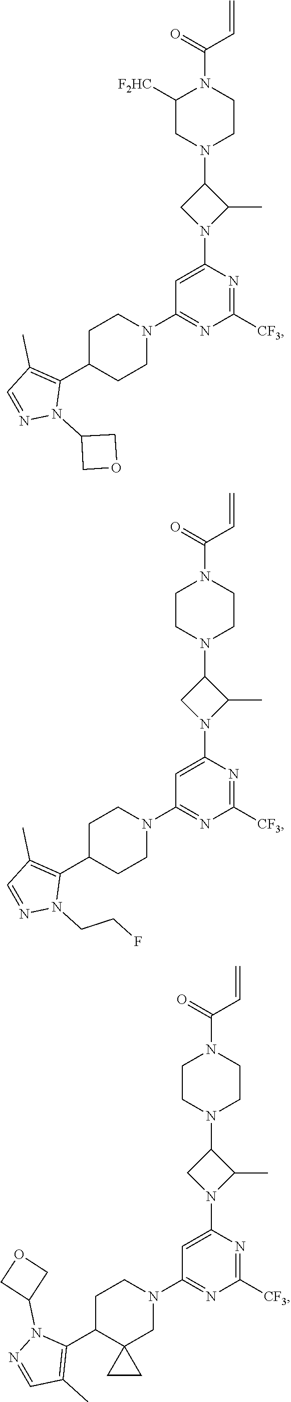 [SMILES: C=CC(=O)N1CCN(C2CN(c3cc(N4CCC(c5c(C)cnn5C5COC5)CC4)nc(C(F)(F)F)n3)C2C)CC1C(F)F ; C=CC(=O)N1CCN(C2CN(c3cc(N4CCC(c5c(C)cnn5CCF)CC4)nc(C(F)(F)F)n3)C2C)CC1 ; C=CC(=O)N1CCN(C2CN(c3cc(N4CCC(c5c(C)cnn5C5COC5)C5(CC5)C4)nc(C(F)(F)F)n3)C2C)CC1]

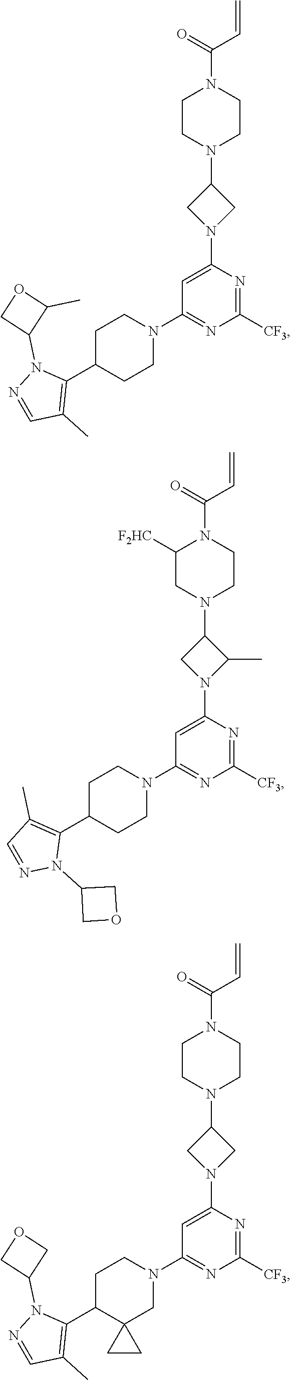 [SMILES: C=CC(=O)N1CCN(C2CN(c3cc(N4CCC(c5c(C)cnn5C5COC5C)CC4)nc(C(F)(F)F)n3)C2)CC1 ; C=CC(=O)N1CCN(C2CN(c3cc(N4CCC(c5c(C)cnn5C5COC5)CC4)nc(C(F)(F)F)n3)C2C)CC1C(F)F ; C=CC(=O)N1CCN(C2CN(c3cc(N4CCC(c5c(C)cnn5C5COC5)C5(CC5)C4)nc(C(F)(F)F)n3)C2)CC1]

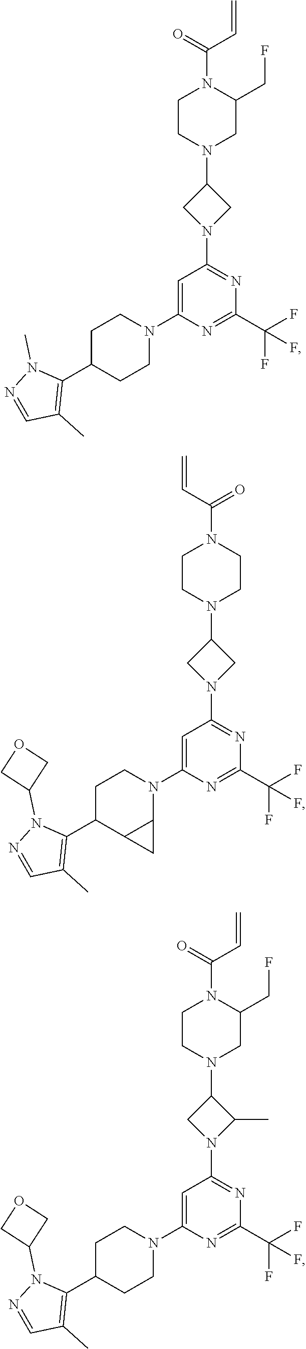 [SMILES: C=CC(=O)N1CCN(C2CN(c3cc(N4CCC(c5c(C)cnn5C)CC4)nc(C(F)(F)F)n3)C2)CC1CF ; C=CC(=O)N1CCN(C2CN(c3cc(N4CCC(c5c(C)cnn5C5COC5)C5CC54)nc(C(F)(F)F)n3)C2)CC1 ; C=CC(=O)N1CCN(C2CN(c3cc(N4CCC(c5c(C)cnn5C5COC5)CC4)nc(C(F)(F)F)n3)C2C)CC1CF]

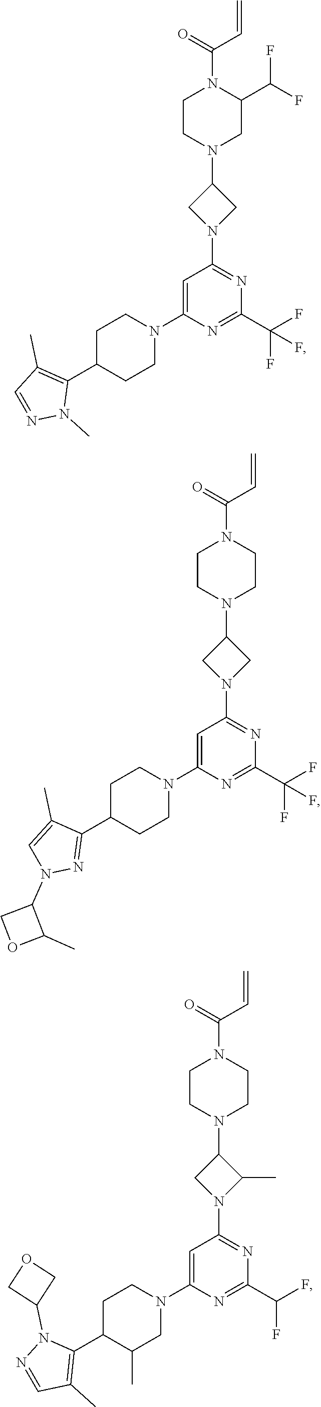 [SMILES: C=CC(=O)N1CCN(C2CN(c3cc(N4CCC(c5c(C)cnn5C)CC4)nc(C(F)(F)F)n3)C2)CC1C(F)F ; C=CC(=O)N1CCN(C2CN(c3cc(N4CCC(c5nn(C6COC6C)cc5C)CC4)nc(C(F)(F)F)n3)C2)CC1 ; C=CC(=O)N1CCN(C2CN(c3cc(N4CCC(c5c(C)cnn5C5COC5)C(C)C4)nc(C(F)F)n3)C2C)CC1]

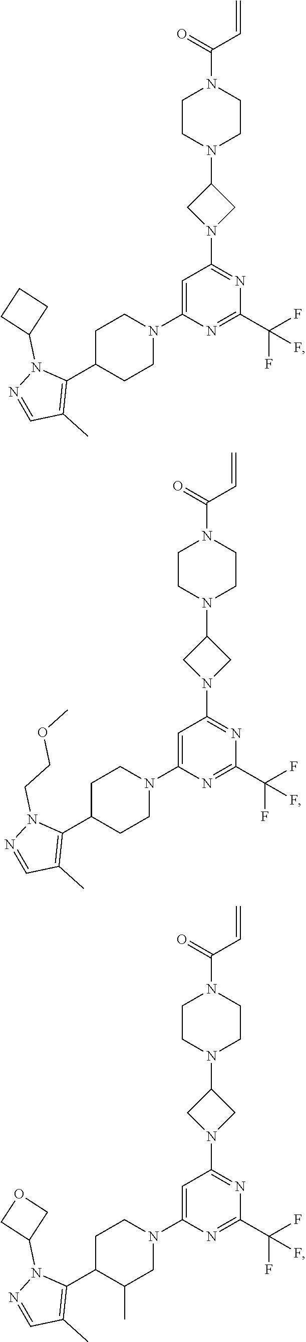 [SMILES: C=CC(=O)N1CCN(C2CN(c3cc(N4CCC(c5c(C)cnn5C5CCC5)CC4)nc(C(F)(F)F)n3)C2)CC1 ; C=CC(=O)N1CCN(C2CN(c3cc(N4CCC(c5c(C)cnn5CCOC)CC4)nc(C(F)(F)F)n3)C2)CC1 ; C=CC(=O)N1CCN(C2CN(c3cc(N4CCC(c5c(C)cnn5C5COC5)C(C)C4)nc(C(F)(F)F)n3)C2)CC1]

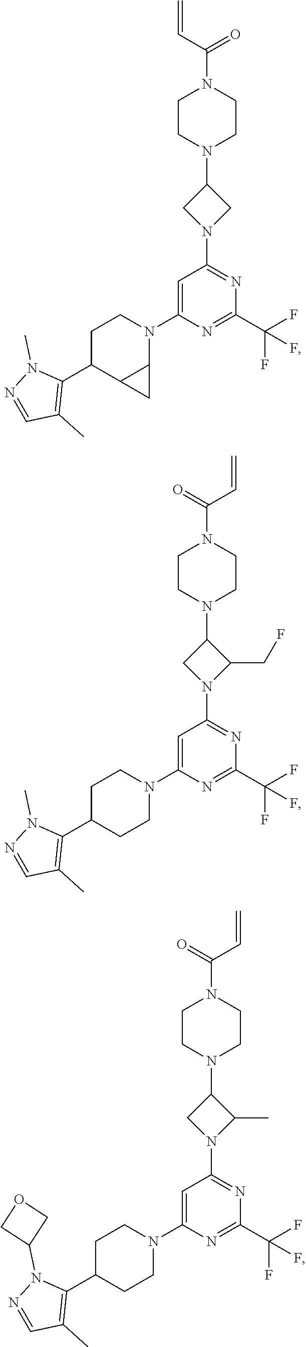 [SMILES: C=CC(=O)N1CCN(C2CN(c3cc(N4CCC(c5c(C)cnn5C)C5CC54)nc(C(F)(F)F)n3)C2)CC1 ; C=CC(=O)N1CCN(C2CN(c3cc(N4CCC(c5c(C)cnn5C)CC4)nc(C(F)(F)F)n3)C2CF)CC1 ; C=CC(=O)N1CCN(C2CN(c3cc(N4CCC(c5c(C)cnn5C5COC5)CC4)nc(C(F)(F)F)n3)C2C)CC1]

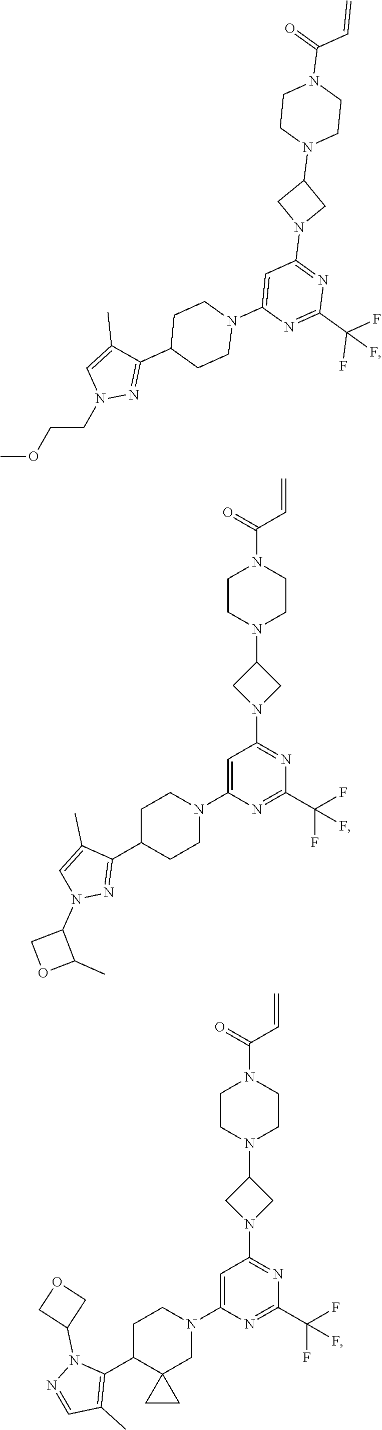 [SMILES: C=CC(=O)N1CCN(C2CN(c3cc(N4CCC(c5nn(CCOC)cc5C)CC4)nc(C(F)(F)F)n3)C2)CC1 ; C=CC(=O)N1CCN(C2CN(c3cc(N4CCC(c5nn(C6COC6C)cc5C)CC4)nc(C(F)(F)F)n3)C2)CC1 ; C=CC(=O)N1CCN(C2CN(c3cc(N4CCC(c5c(C)cnn5C5COC5)C5(CC5)C4)nc(C(F)(F)F)n3)C2)CC1]

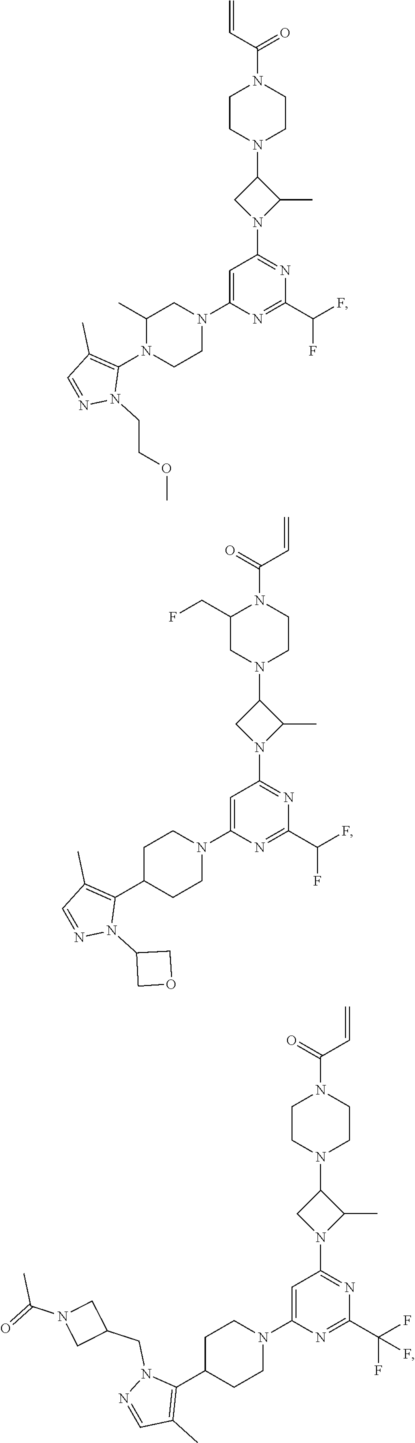 [SMILES: C=CC(=O)N1CCN(C2CN(c3cc(N4CCN(c5c(C)cnn5CCOC)C(C)C4)nc(C(F)F)n3)C2C)CC1 ; C=CC(=O)N1CCN(C2CN(c3cc(N4CCC(c5c(C)cnn5C5COC5)CC4)nc(C(F)F)n3)C2C)CC1CF ; C=CC(=O)N1CCN(C2CN(c3cc(N4CCC(c5c(C)cnn5CC5CN(C(C)=O)C5)CC4)nc(C(F)(F)F)n3)C2C)CC1]

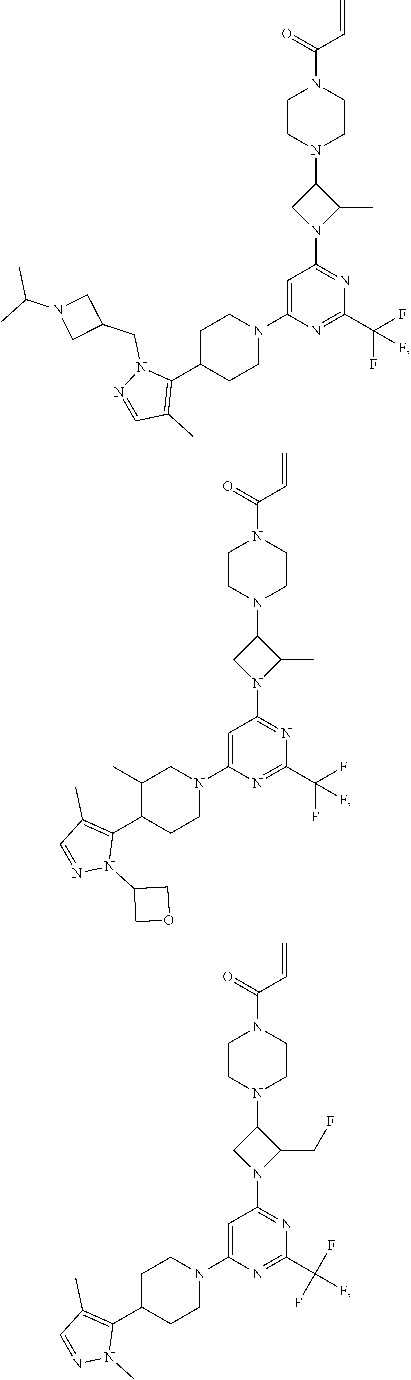 [SMILES: C=CC(=O)N1CCN(C2CN(c3cc(N4CCC(c5c(C)cnn5CC5CN(C(C)C)C5)CC4)nc(C(F)(F)F)n3)C2C)CC1 ; C=CC(=O)N1CCN(C2CN(c3cc(N4CCC(c5c(C)cnn5C5COC5)C(C)C4)nc(C(F)(F)F)n3)C2C)CC1 ; C=CC(=O)N1CCN(C2CN(c3cc(N4CCC(c5c(C)cnn5C)CC4)nc(C(F)(F)F)n3)C2CF)CC1]

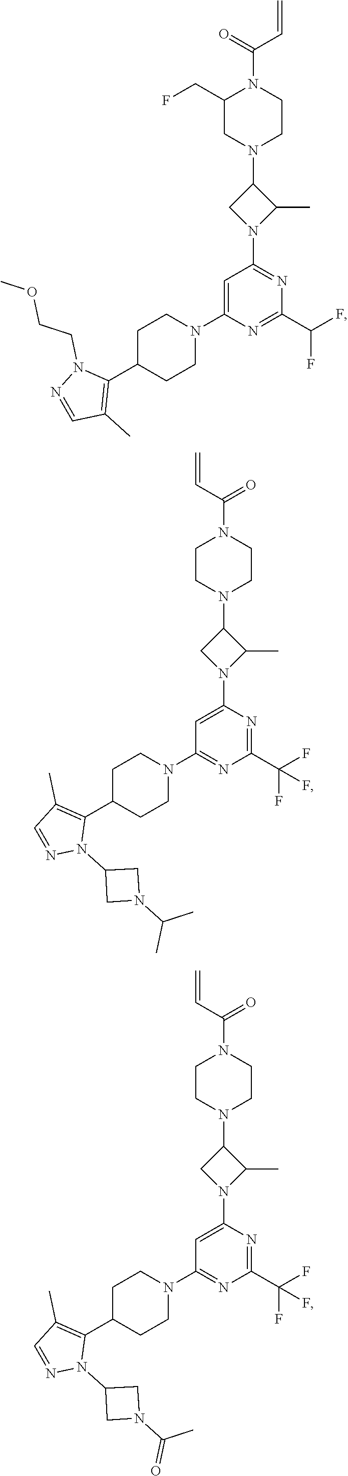

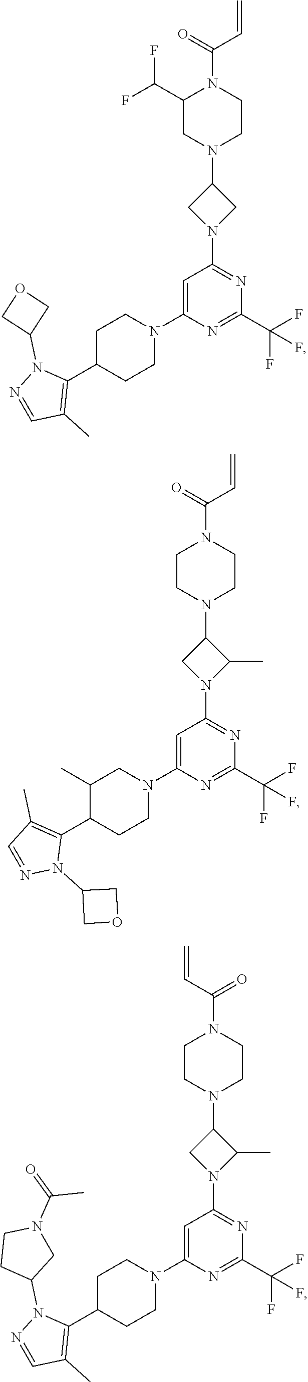 [SMILES: C=CC(=O)N1CCN(C2CN(c3cc(N4CCC(c5c(C)cnn5C5COC5)CC4)nc(C(F)(F)F)n3)C2)CC1C(F)F ; C=CC(=O)N1CCN(C2CN(c3cc(N4CCC(c5c(C)cnn5C5COC5)C(C)C4)nc(C(F)(F)F)n3)C2C)CC1 ; C=CC(=O)N1CCN(C2CN(c3cc(N4CCC(c5c(C)cnn5C5CCN(C(C)=O)C5)CC4)nc(C(F)(F)F)n3)C2C)CC1]

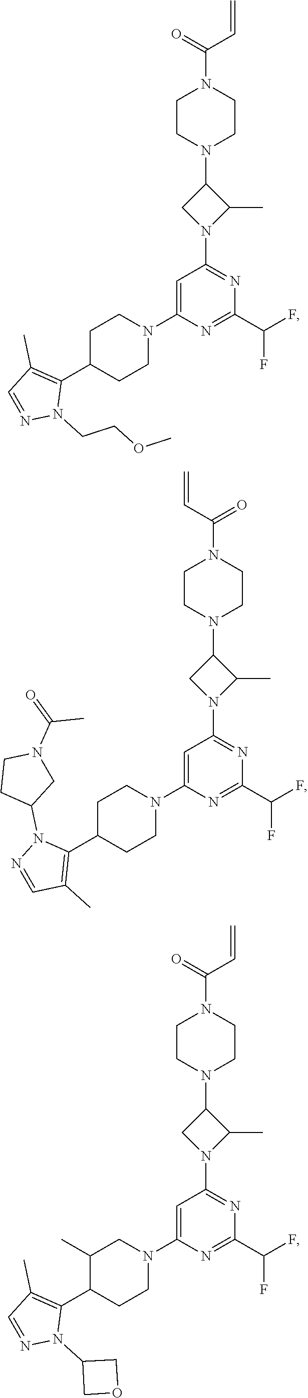

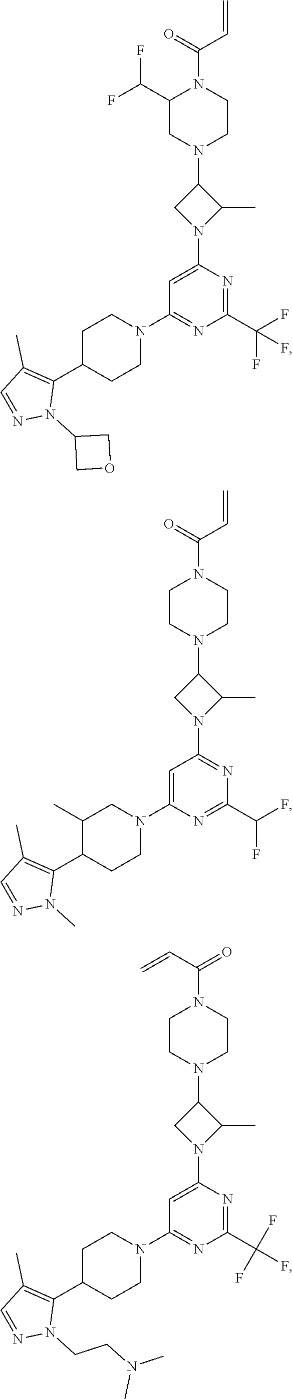 [SMILES: C=CC(=O)N1CCN(C2CN(c3cc(N4CCC(c5c(C)cnn5C5COC5)CC4)nc(C(F)(F)F)n3)C2C)CC1C(F)F ; C=CC(=O)N1CCN(C2CN(c3cc(N4CCC(c5c(C)cnn5C)C(C)C4)nc(C(F)F)n3)C2C)CC1 ; C=CC(=O)N1CCN(C2CN(c3cc(N4CCC(c5c(C)cnn5CCN(C)C)CC4)nc(C(F)(F)F)n3)C2C)CC1]

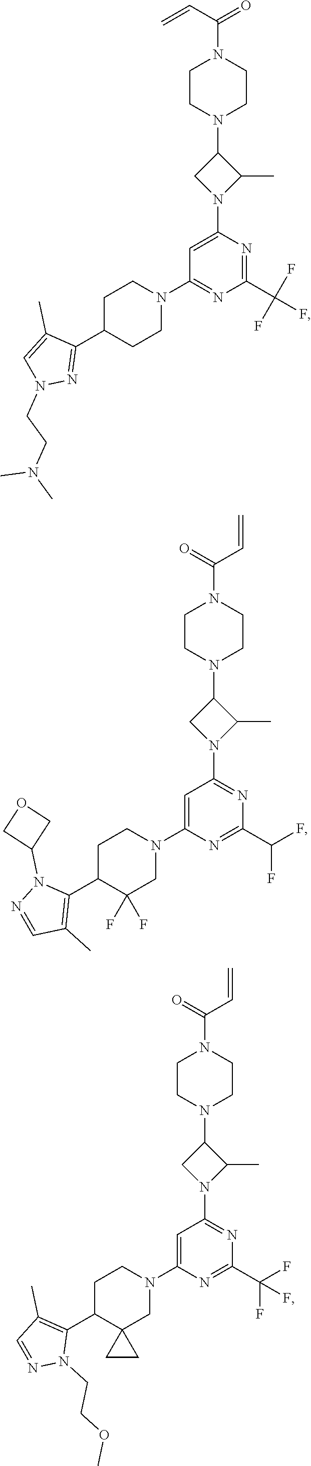 [SMILES: C=CC(=O)N1CCN(C2CN(c3cc(N4CCC(c5nn(CCN(C)C)cc5C)CC4)nc(C(F)(F)F)n3)C2C)CC1 ; C=CC(=O)N1CCN(C2CN(c3cc(N4CCC(c5c(C)cnn5C5COC5)C(F)(F)C4)nc(C(F)F)n3)C2C)CC1 ; C=CC(=O)N1CCN(C2CN(c3cc(N4CCC(c5c(C)cnn5CCOC)C5(CC5)C4)nc(C(F)(F)F)n3)C2C)CC1]

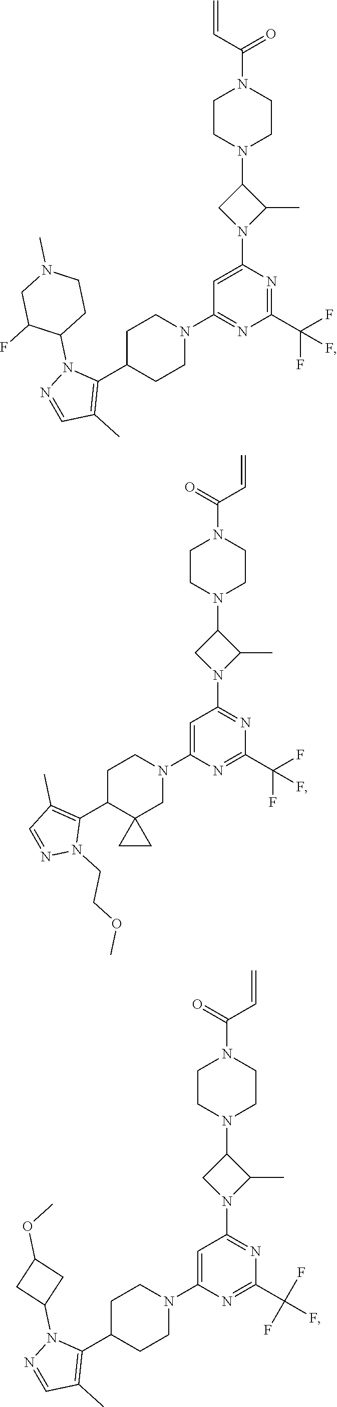 [SMILES: C=CC(=O)N1CCN(C2CN(c3cc(N4CCC(c5c(C)cnn5C5CCN(C)CC5F)CC4)nc(C(F)(F)F)n3)C2C)CC1 ; C=CC(=O)N1CCN(C2CN(c3cc(N4CCC(c5c(C)cnn5CCOC)C5(CC5)C4)nc(C(F)(F)F)n3)C2C)CC1 ; C=CC(=O)N1CCN(C2CN(c3cc(N4CCC(c5c(C)cnn5C5CC(OC)C5)CC4)nc(C(F)(F)F)n3)C2C)CC1]

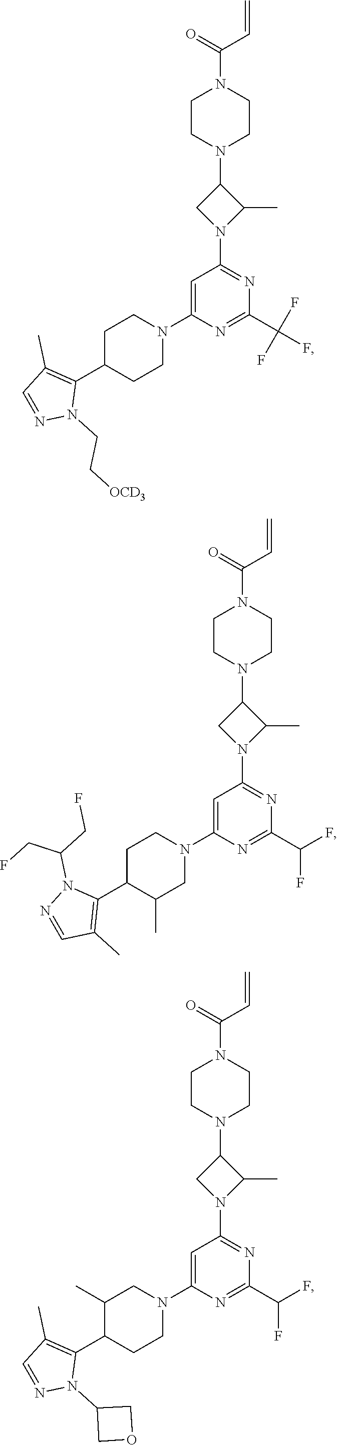

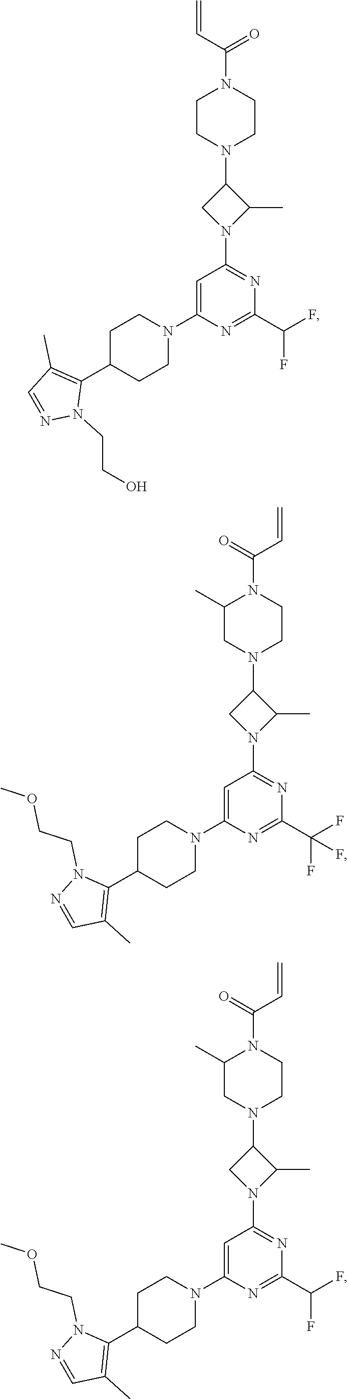 [SMILES: C=CC(=O)N1CCN(C2CN(c3cc(N4CCC(c5c(C)cnn5CCO)CC4)nc(C(F)F)n3)C2C)CC1 ; C=CC(=O)N1CCN(C2CN(c3cc(N4CCC(c5c(C)cnn5CCOC)CC4)nc(C(F)(F)F)n3)C2C)CC1C ; C=CC(=O)N1CCN(C2CN(c3cc(N4CCC(c5c(C)cnn5CCOC)CC4)nc(C(F)F)n3)C2C)CC1C]

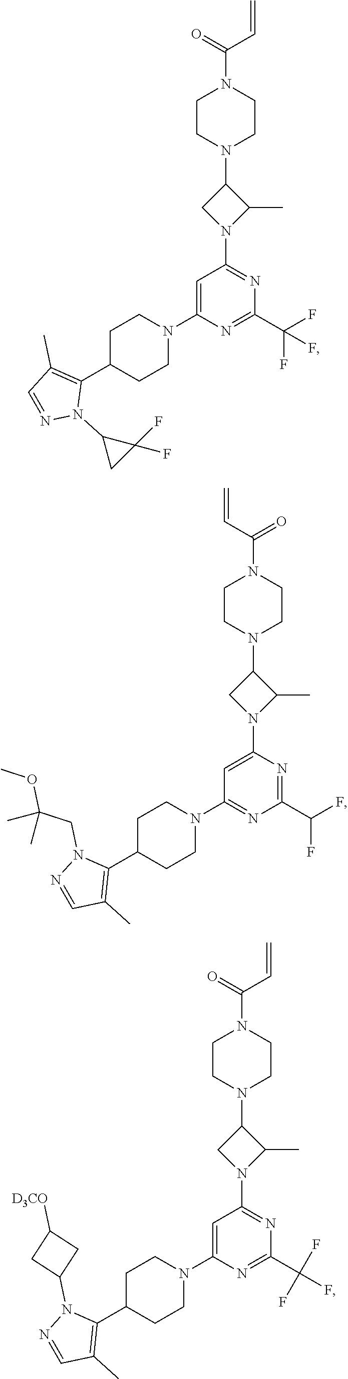

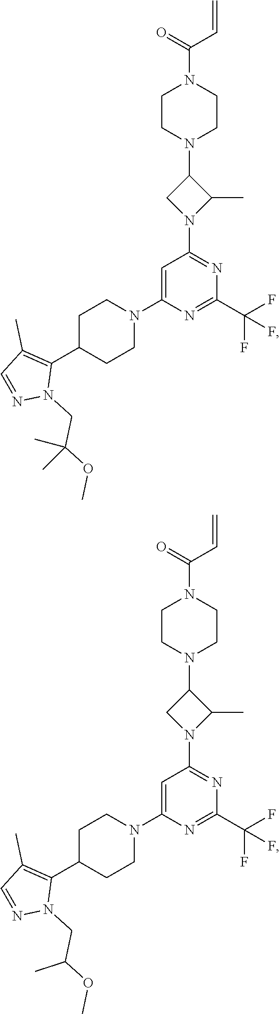 [SMILES: C=CC(=O)N1CCN(C2CN(c3cc(N4CCC(c5c(C)cnn5CC(C)(C)OC)CC4)nc(C(F)(F)F)n3)C2C)CC1 ; C=CC(=O)N1CCN(C2CN(c3cc(N4CCC(c5c(C)cnn5CC(C)OC)CC4)nc(C(F)(F)F)n3)C2C)CC1]

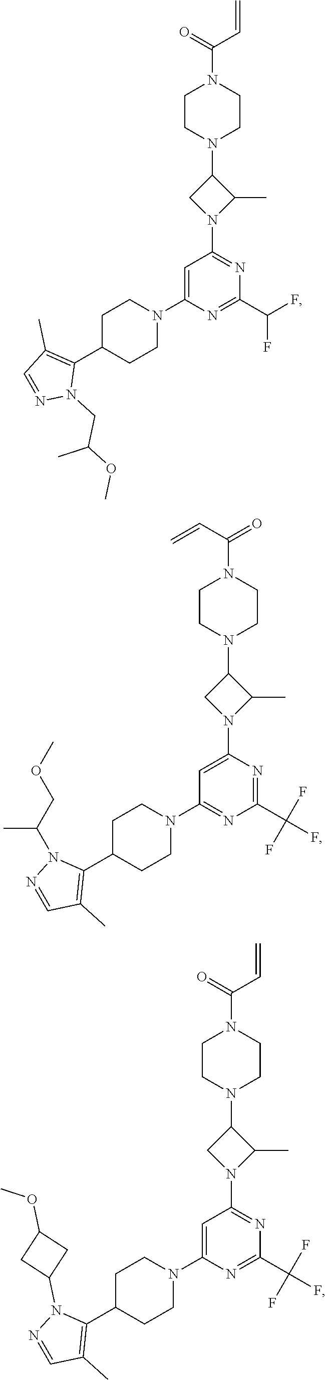 [SMILES: C=CC(=O)N1CCN(C2CN(c3cc(N4CCC(c5c(C)cnn5CC(C)OC)CC4)nc(C(F)F)n3)C2C)CC1 ; C=CC(=O)N1CCN(C2CN(c3cc(N4CCC(c5c(C)cnn5C(C)COC)CC4)nc(C(F)(F)F)n3)C2C)CC1 ; C=CC(=O)N1CCN(C2CN(c3cc(N4CCC(c5c(C)cnn5C5CC(OC)C5)CC4)nc(C(F)(F)F)n3)C2C)CC1]

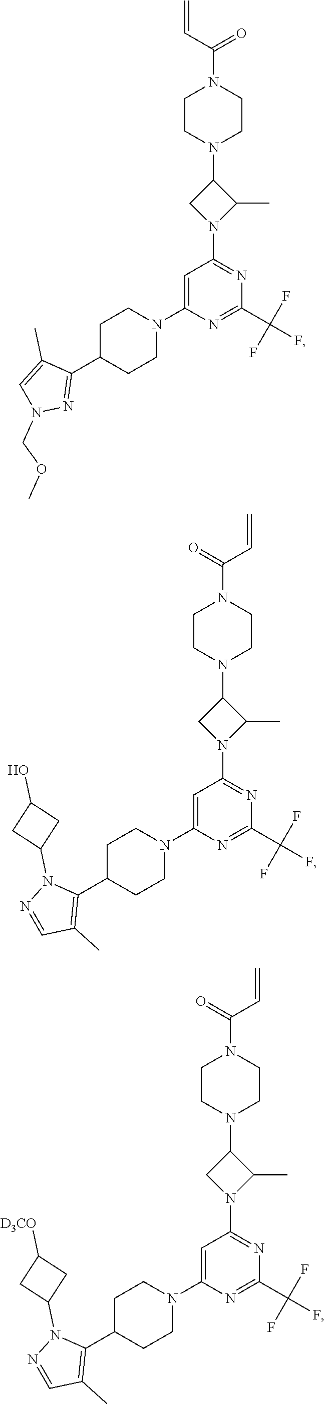

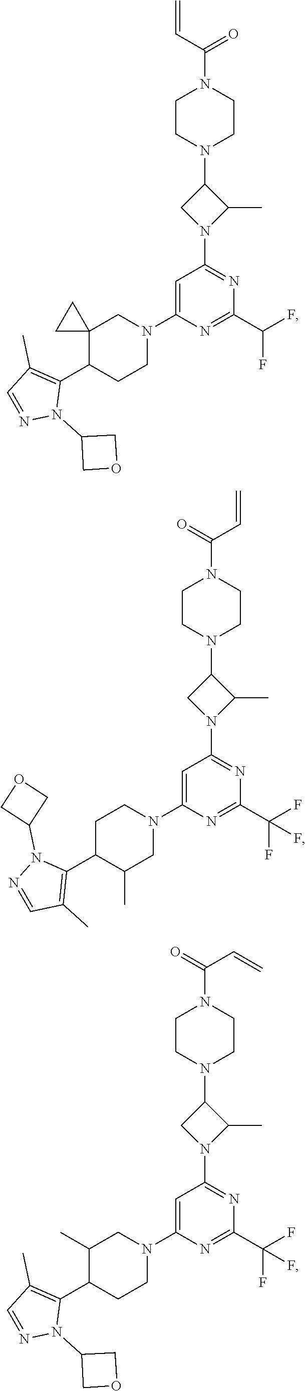 [SMILES: C=CC(=O)N1CCN(C2CN(c3cc(N4CCC(c5c(C)cnn5C5COC5)C5(CC5)C4)nc(C(F)F)n3)C2C)CC1 ; C=CC(=O)N1CCN(C2CN(c3cc(N4CCC(c5c(C)cnn5C5COC5)C(C)C4)nc(C(F)(F)F)n3)C2C)CC1 ; C=CC(=O)N1CCN(C2CN(c3cc(N4CCC(c5c(C)cnn5C5COC5)C(C)C4)nc(C(F)(F)F)n3)C2C)CC1]

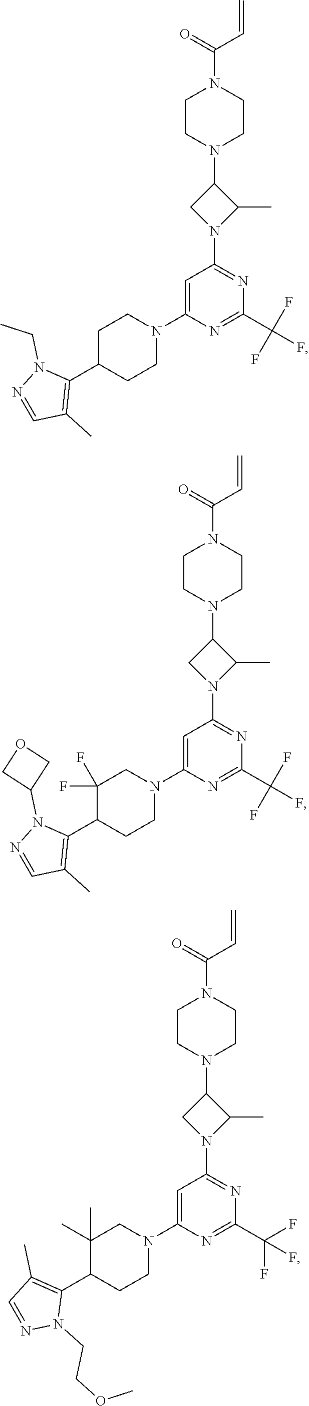 [SMILES: C=CC(=O)N1CCN(C2CN(c3cc(N4CCC(c5c(C)cnn5CC)CC4)nc(C(F)(F)F)n3)C2C)CC1 ; C=CC(=O)N1CCN(C2CN(c3cc(N4CCC(c5c(C)cnn5C5COC5)C(F)(F)C4)nc(C(F)(F)F)n3)C2C)CC1 ; C=CC(=O)N1CCN(C2CN(c3cc(N4CCC(c5c(C)cnn5CCOC)C(C)(C)C4)nc(C(F)(F)F)n3)C2C)CC1]

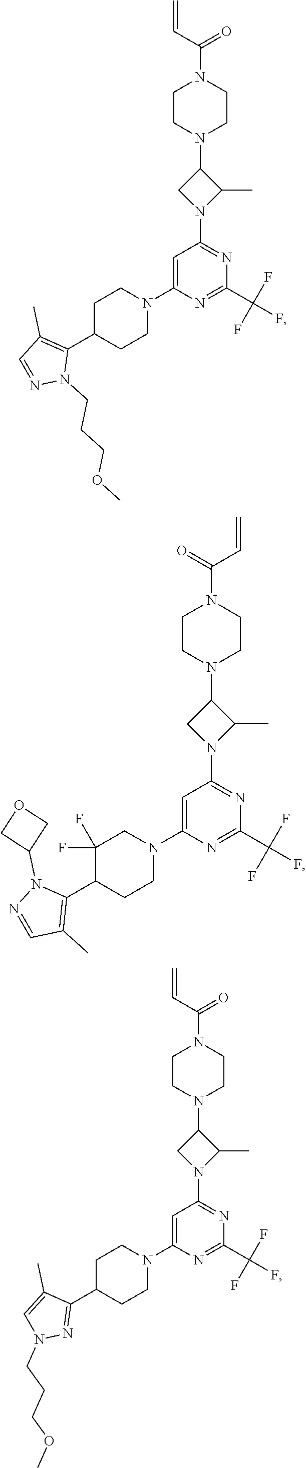 [SMILES: C=CC(=O)N1CCN(C2CN(c3cc(N4CCC(c5c(C)cnn5CCCOC)CC4)nc(C(F)(F)F)n3)C2C)CC1 ; C=CC(=O)N1CCN(C2CN(c3cc(N4CCC(c5c(C)cnn5C5COC5)C(F)(F)C4)nc(C(F)(F)F)n3)C2C)CC1 ; C=CC(=O)N1CCN(C2CN(c3cc(N4CCC(c5nn(CCCOC)cc5C)CC4)nc(C(F)(F)F)n3)C2C)CC1]

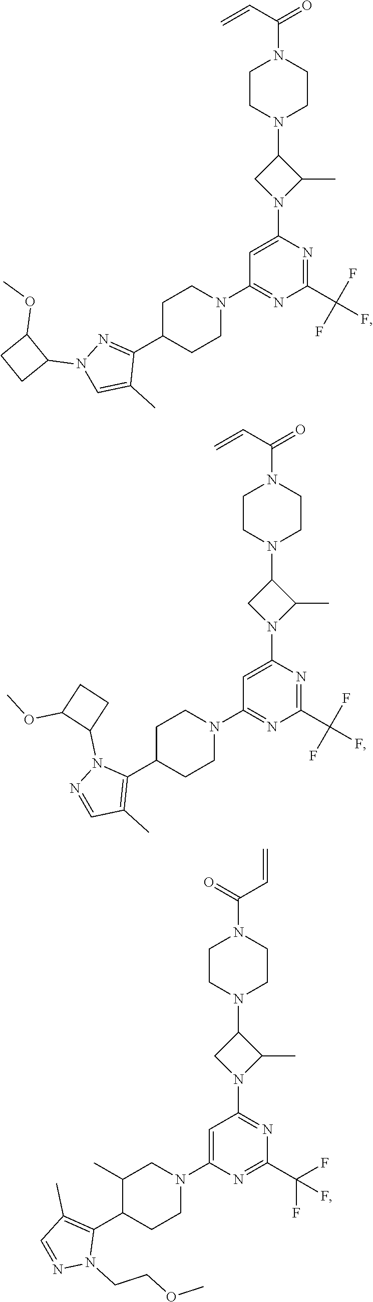 [SMILES: C=CC(=O)N1CCN(C2CN(c3cc(N4CCC(c5nn(C6CCC6OC)cc5C)CC4)nc(C(F)(F)F)n3)C2C)CC1 ; C=CC(=O)N1CCN(C2CN(c3cc(N4CCC(c5c(C)cnn5C5CCC5OC)CC4)nc(C(F)(F)F)n3)C2C)CC1 ; C=CC(=O)N1CCN(C2CN(c3cc(N4CCC(c5c(C)cnn5CCOC)C(C)C4)nc(C(F)(F)F)n3)C2C)CC1]

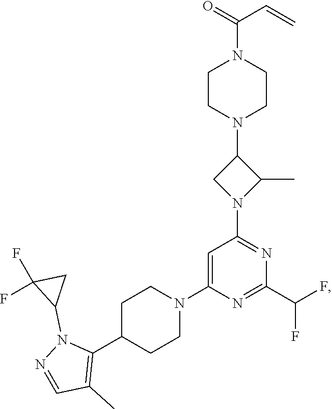 [SMILES: C=CC(=O)N1CCN(C2CN(c3cc(N4CCC(c5c(C)cnn5C5CC5(F)F)CC4)nc(C(F)F)n3)C2C)CC1]

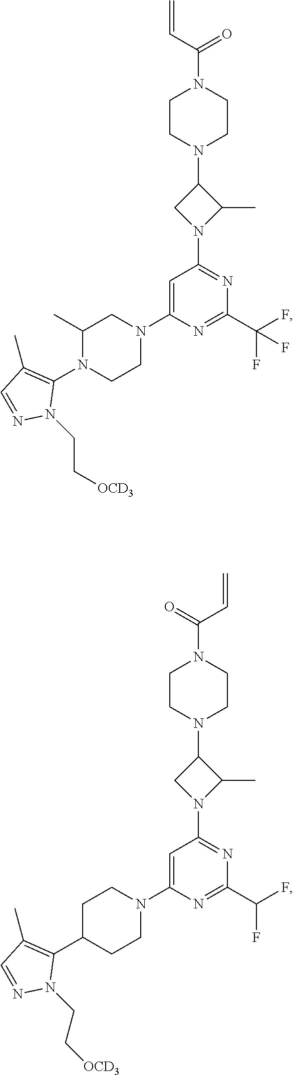

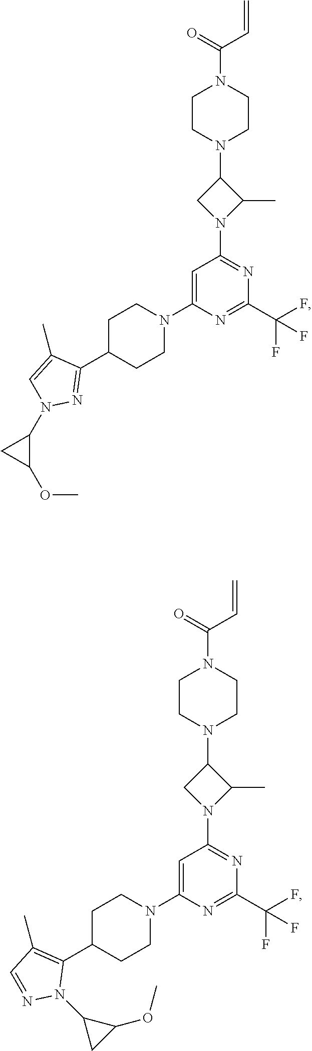 [SMILES: C=CC(=O)N1CCN(C2CN(c3cc(N4CCC(c5nn(C6CC6OC)cc5C)CC4)nc(C(F)(F)F)n3)C2C)CC1 ; C=CC(=O)N1CCN(C2CN(c3cc(N4CCC(c5c(C)cnn5C5CC5OC)CC4)nc(C(F)(F)F)n3)C2C)CC1]

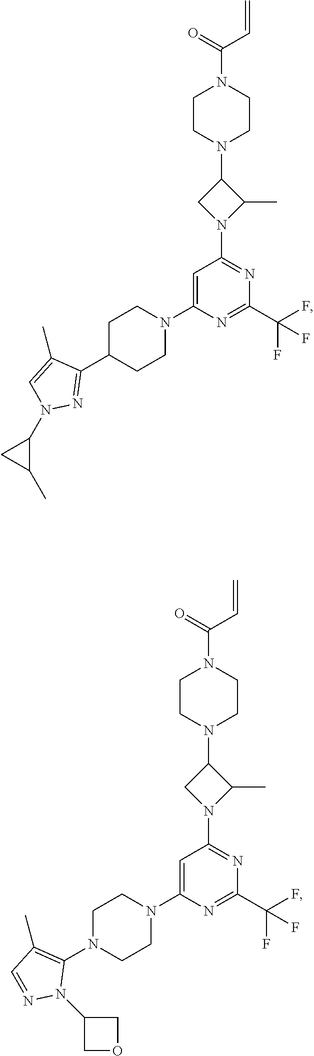 [SMILES: C=CC(=O)N1CCN(C2CN(c3cc(N4CCC(c5nn(C6CC6C)cc5C)CC4)nc(C(F)(F)F)n3)C2C)CC1 ; C=CC(=O)N1CCN(C2CN(c3cc(N4CCN(c5c(C)cnn5C5COC5)CC4)nc(C(F)(F)F)n3)C2C)CC1]

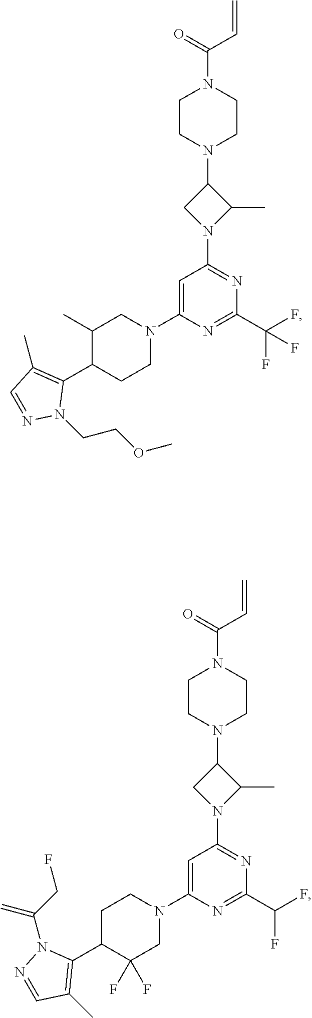 [SMILES: C=CC(=O)N1CCN(C2CN(c3cc(N4CCC(c5c(C)cnn5CCOC)C(C)C4)nc(C(F)(F)F)n3)C2C)CC1 ; C=CC(=O)N1CCN(C2CN(c3cc(N4CCC(c5c(C)cnn5C(=C)CF)C(F)(F)C4)nc(C(F)F)n3)C2C)CC1]

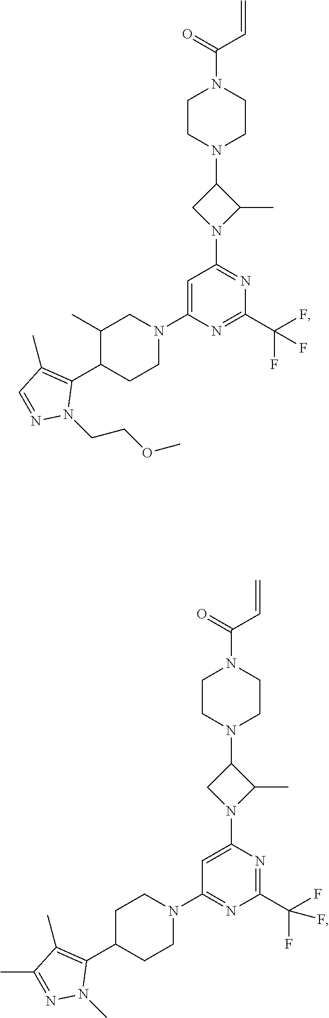 [SMILES: C=CC(=O)N1CCN(C2CN(c3cc(N4CCC(c5c(C)cnn5CCOC)C(C)C4)nc(C(F)(F)F)n3)C2C)CC1 ; C=CC(=O)N1CCN(C2CN(c3cc(N4CCC(c5c(C)c(C)nn5C)CC4)nc(C(F)(F)F)n3)C2C)CC1]

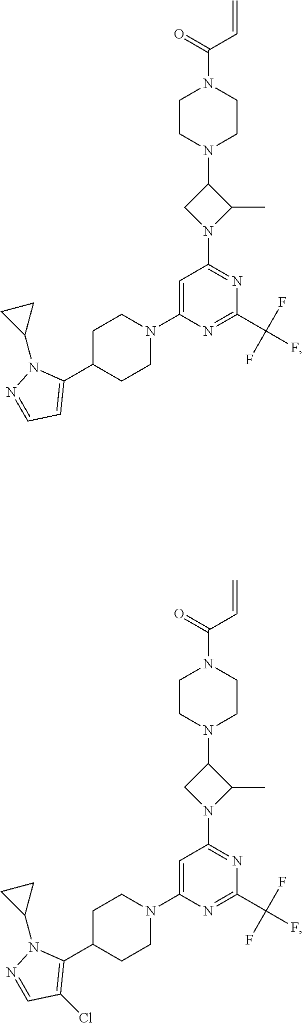 [SMILES: C=CC(=O)N1CCN(C2CN(c3cc(N4CCC(c5ccnn5C5CC5)CC4)nc(C(F)(F)F)n3)C2C)CC1 ; C=CC(=O)N1CCN(C2CN(c3cc(N4CCC(c5c(Cl)cnn5C5CC5)CC4)nc(C(F)(F)F)n3)C2C)CC1]

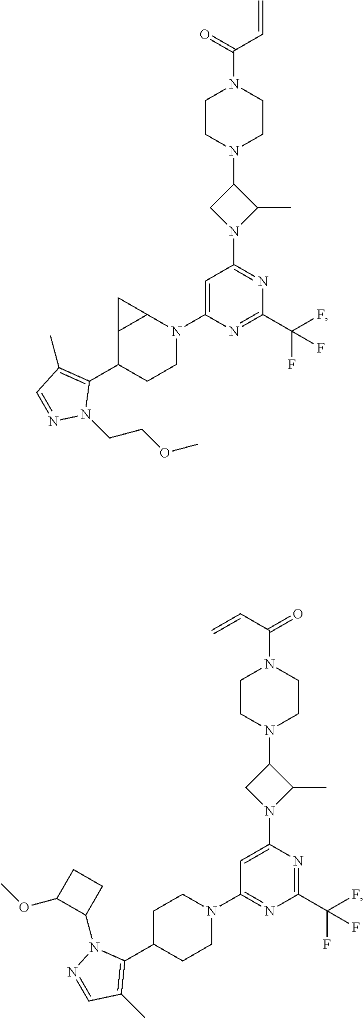 [SMILES: C=CC(=O)N1CCN(C2CN(c3cc(N4CCC(c5c(C)cnn5CCOC)C5CC54)nc(C(F)(F)F)n3)C2C)CC1 ; C=CC(=O)N1CCN(C2CN(c3cc(N4CCC(c5c(C)cnn5C5CCC5OC)CC4)nc(C(F)(F)F)n3)C2C)CC1]

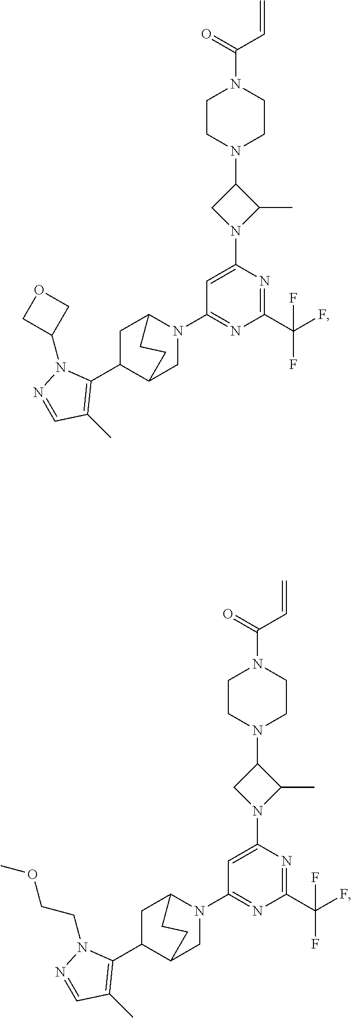 [SMILES: C=CC(=O)N1CCN(C2CN(c3cc(N4CC5CCC4CC5c4c(C)cnn4C4COC4)nc(C(F)(F)F)n3)C2C)CC1 ; C=CC(=O)N1CCN(C2CN(c3cc(N4CC5CCC4CC5c4c(C)cnn4CCOC)nc(C(F)(F)F)n3)C2C)CC1]

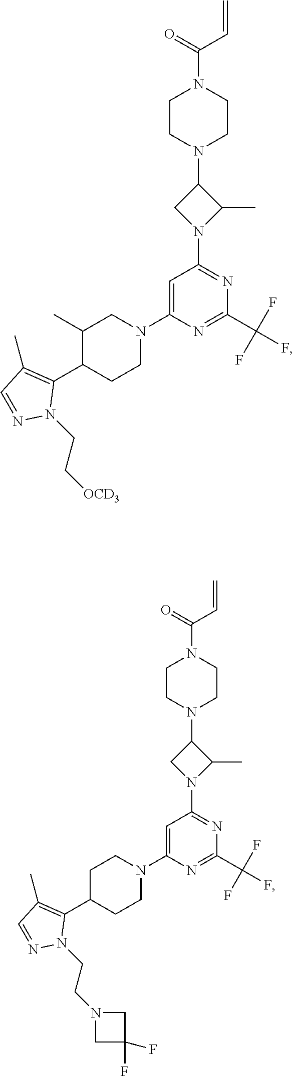

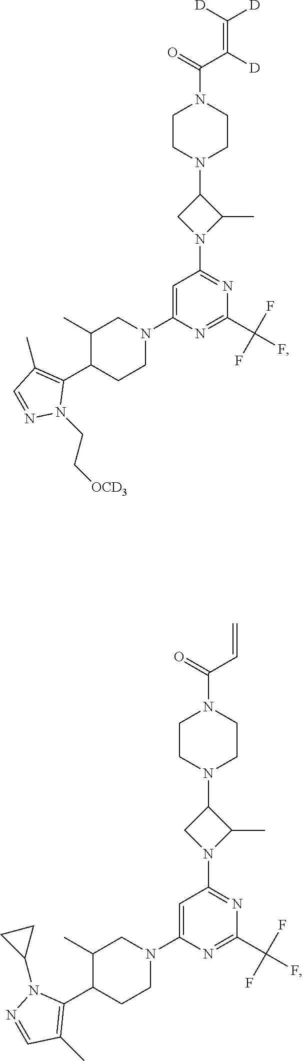

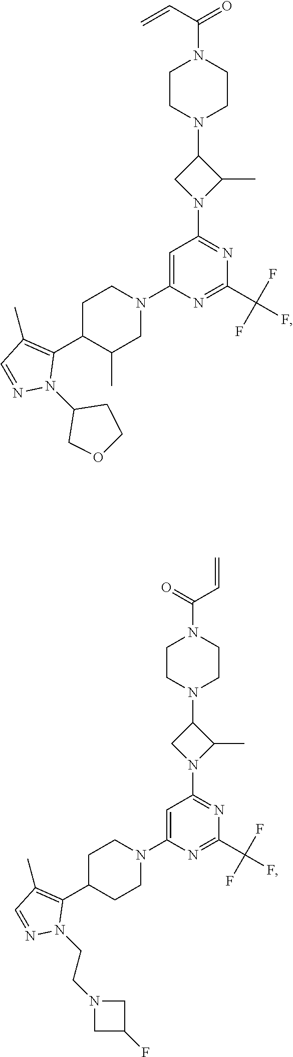 [SMILES: C=CC(=O)N1CCN(C2CN(c3cc(N4CCC(c5c(C)cnn5C5CCOC5)C(C)C4)nc(C(F)(F)F)n3)C2C)CC1 ; C=CC(=O)N1CCN(C2CN(c3cc(N4CCC(c5c(C)cnn5CCN5CC(F)C5)CC4)nc(C(F)(F)F)n3)C2C)CC1]

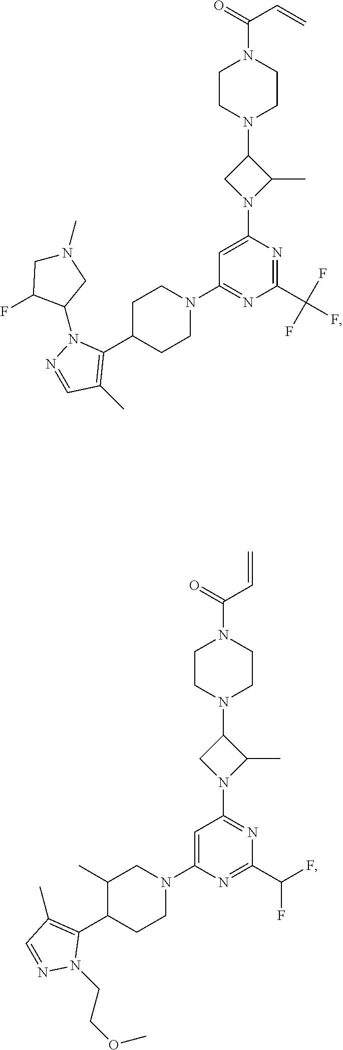 [SMILES: C=CC(=O)N1CCN(C2CN(c3cc(N4CCC(c5c(C)cnn5C5CN(C)CC5F)CC4)nc(C(F)(F)F)n3)C2C)CC1 ; C=CC(=O)N1CCN(C2CN(c3cc(N4CCC(c5c(C)cnn5CCOC)C(C)C4)nc(C(F)F)n3)C2C)CC1]

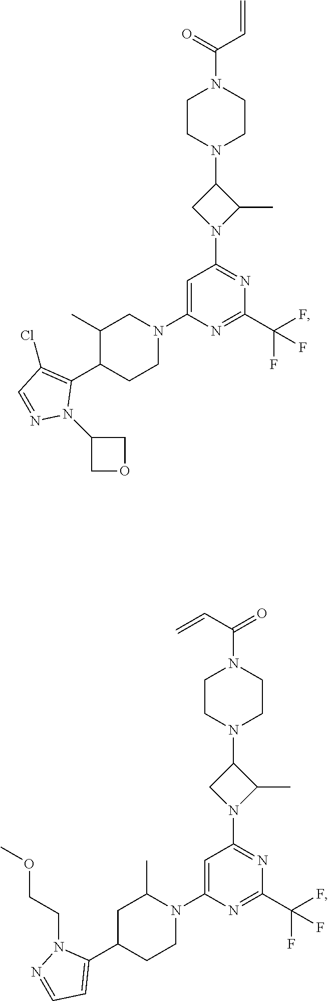 [SMILES: C=CC(=O)N1CCN(C2CN(c3cc(N4CCC(c5c(Cl)cnn5C5COC5)C(C)C4)nc(C(F)(F)F)n3)C2C)CC1 ; C=CC(=O)N1CCN(C2CN(c3cc(N4CCC(c5ccnn5CCOC)CC4C)nc(C(F)(F)F)n3)C2C)CC1]

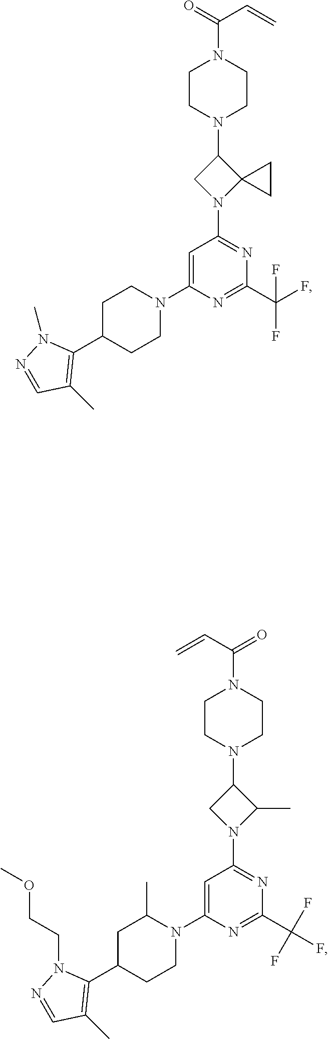 [SMILES: C=CC(=O)N1CCN(C2CN(c3cc(N4CCC(c5c(C)cnn5C)CC4)nc(C(F)(F)F)n3)C23CC3)CC1 ; C=CC(=O)N1CCN(C2CN(c3cc(N4CCC(c5c(C)cnn5CCOC)CC4C)nc(C(F)(F)F)n3)C2C)CC1]

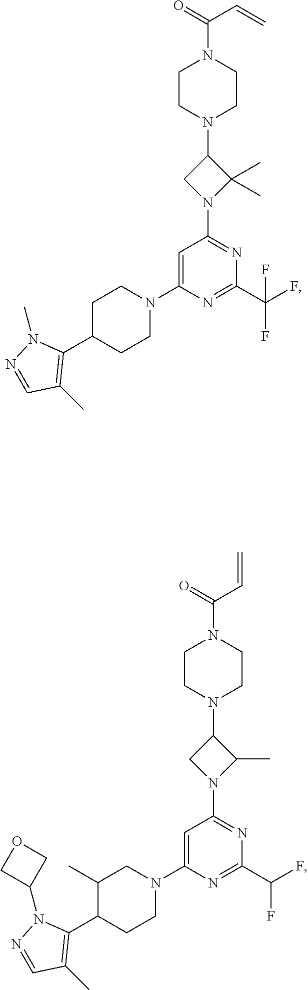 [SMILES: C=CC(=O)N1CCN(C2CN(c3cc(N4CCC(c5c(C)cnn5C)CC4)nc(C(F)(F)F)n3)C2(C)C)CC1 ; C=CC(=O)N1CCN(C2CN(c3cc(N4CCC(c5c(C)cnn5C5COC5)C(C)C4)nc(C(F)F)n3)C2C)CC1]

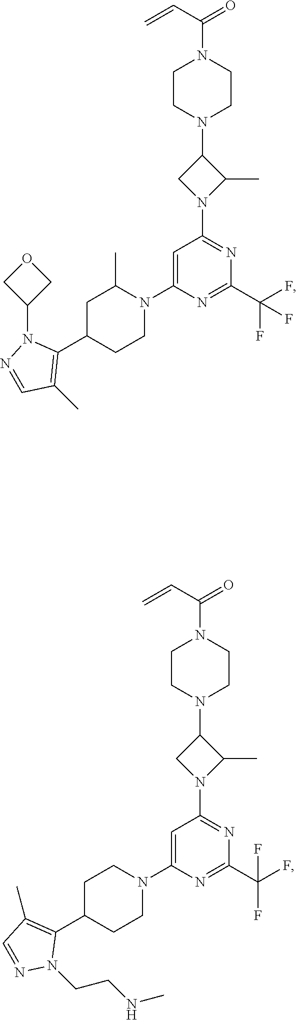 [SMILES: C=CC(=O)N1CCN(C2CN(c3cc(N4CCC(c5c(C)cnn5C5COC5)CC4C)nc(C(F)(F)F)n3)C2C)CC1 ; C=CC(=O)N1CCN(C2CN(c3cc(N4CCC(c5c(C)cnn5CCNC)CC4)nc(C(F)(F)F)n3)C2C)CC1]

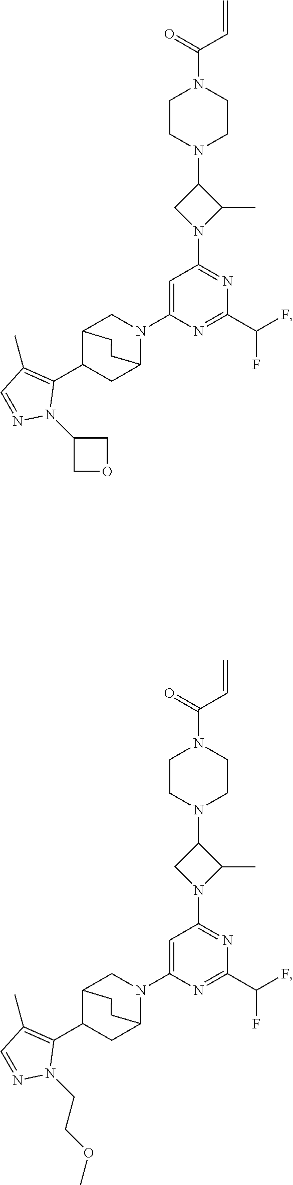 [SMILES: C=CC(=O)N1CCN(C2CN(c3cc(N4CC5CCC4CC5c4c(C)cnn4C4COC4)nc(C(F)F)n3)C2C)CC1 ; C=CC(=O)N1CCN(C2CN(c3cc(N4CC5CCC4CC5c4c(C)cnn4CCOC)nc(C(F)F)n3)C2C)CC1]

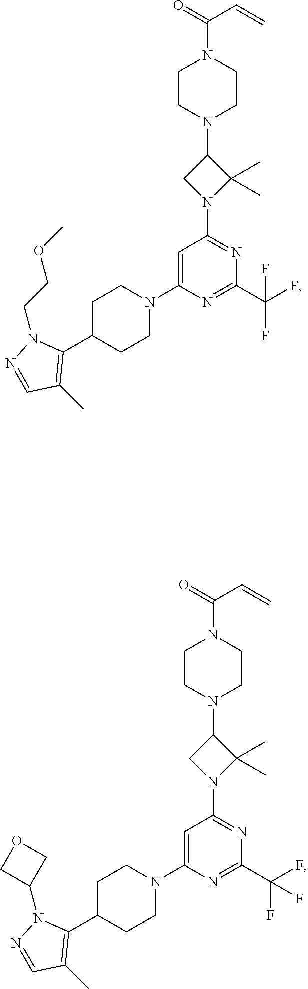 [SMILES: C=CC(=O)N1CCN(C2CN(c3cc(N4CCC(c5c(C)cnn5CCOC)CC4)nc(C(F)(F)F)n3)C2(C)C)CC1 ; C=CC(=O)N1CCN(C2CN(c3cc(N4CCC(c5c(C)cnn5C5COC5)CC4)nc(C(F)(F)F)n3)C2(C)C)CC1]

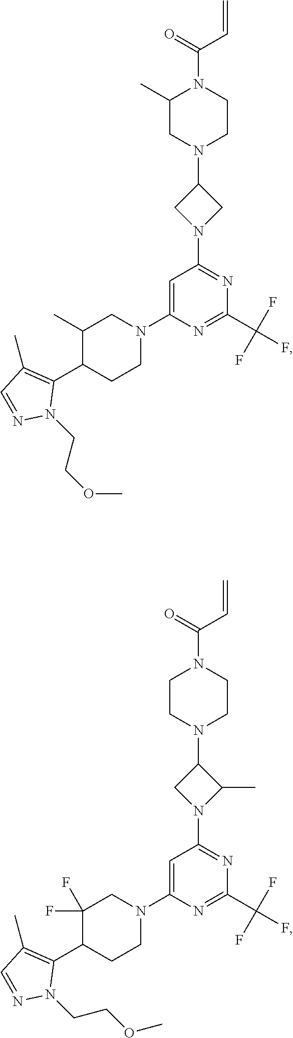 [SMILES: C=CC(=O)N1CCN(C2CN(c3cc(N4CCC(c5c(C)cnn5CCOC)C(C)C4)nc(C(F)(F)F)n3)C2)CC1C ; C=CC(=O)N1CCN(C2CN(c3cc(N4CCC(c5c(C)cnn5CCOC)C(F)(F)C4)nc(C(F)(F)F)n3)C2C)CC1]

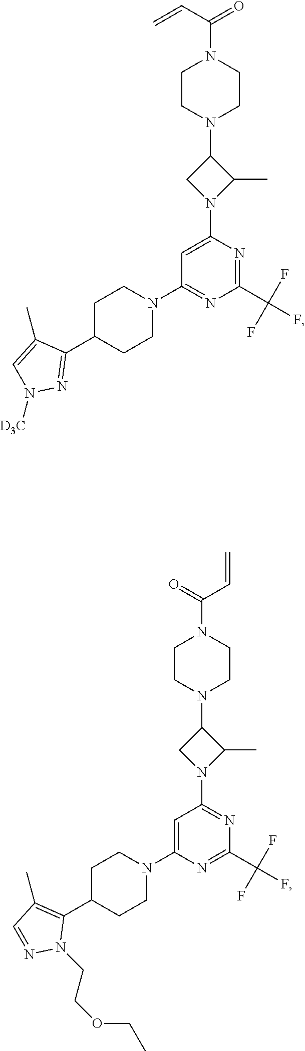 [SMILES: [2H]C([2H])([2H])n1cc(C)c(C2CCN(c3cc(N4CC(N5CCN(C(=O)C=C)CC5)C4C)nc(C(F)(F)F)n3)CC2)n1 ; C=CC(=O)N1CCN(C2CN(c3cc(N4CCC(c5c(C)cnn5CCOCC)CC4)nc(C(F)(F)F)n3)C2C)CC1]

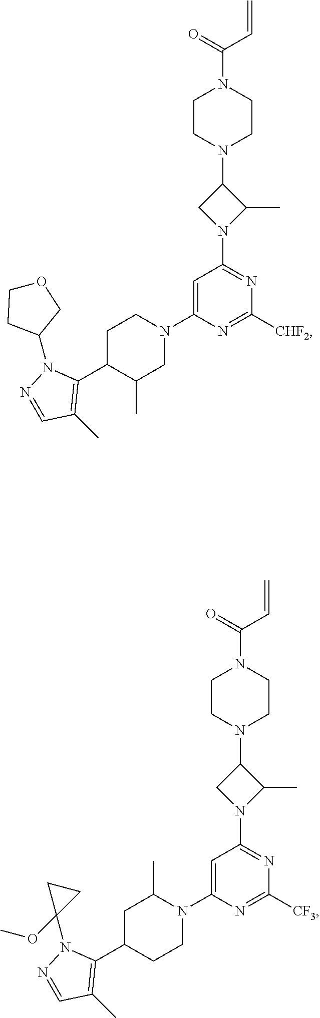 [SMILES: C=CC(=O)N1CCN(C2CN(c3cc(N4CCC(c5c(C)cnn5C5CCOC5)C(C)C4)nc(C(F)F)n3)C2C)CC1 ; C=CC(=O)N1CCN(C2CN(c3cc(N4CCC(c5c(C)cnn5C5(OC)CC5)CC4C)nc(C(F)(F)F)n3)C2C)CC1]

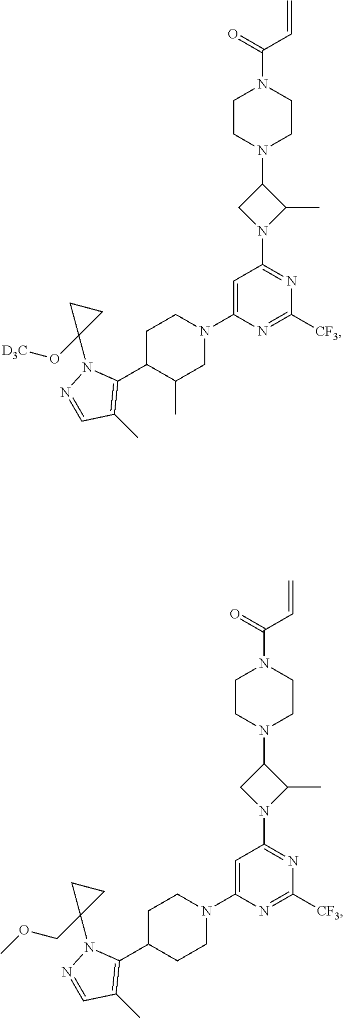 [SMILES: [2H]C([2H])([2H])OC1(n2ncc(C)c2C2CCN(c3cc(N4CC(N5CCN(C(=O)C=C)CC5)C4C)nc(C(F)(F)F)n3)CC2C)CC1 ; C=CC(=O)N1CCN(C2CN(c3cc(N4CCC(c5c(C)cnn5C5(COC)CC5)CC4)nc(C(F)(F)F)n3)C2C)CC1]

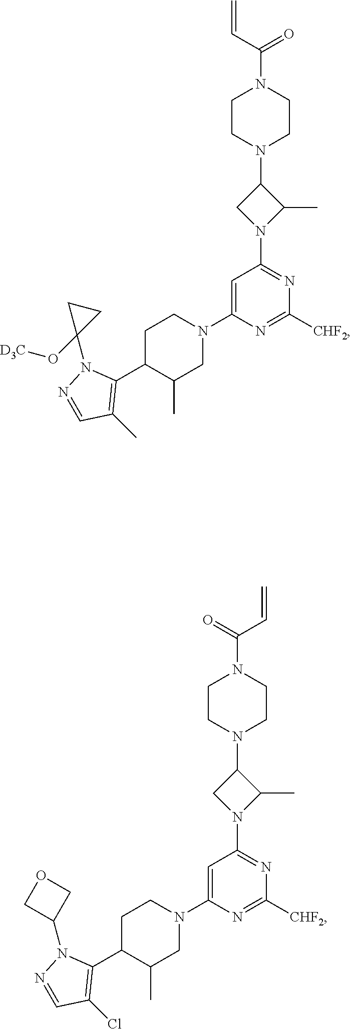 [SMILES: [2H]C([2H])([2H])OC1(n2ncc(C)c2C2CCN(c3cc(N4CC(N5CCN(C(=O)C=C)CC5)C4C)nc(C(F)F)n3)CC2C)CC1 ; C=CC(=O)N1CCN(C2CN(c3cc(N4CCC(c5c(Cl)cnn5C5COC5)C(C)C4)nc(C(F)F)n3)C2C)CC1]

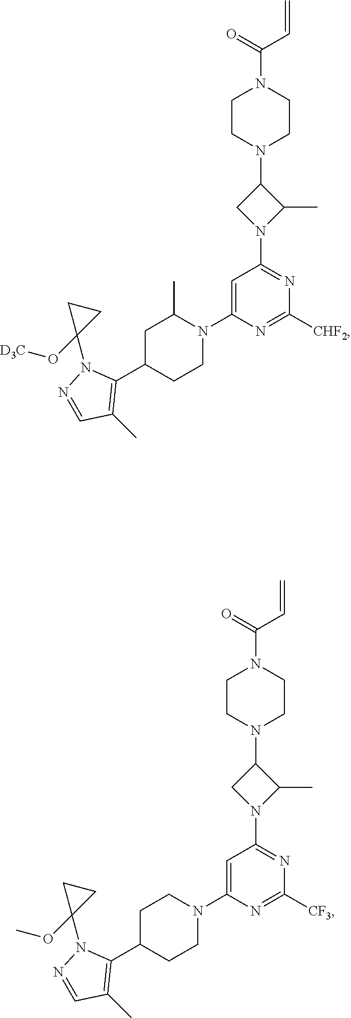 [SMILES: [2H]C([2H])([2H])OC1(n2ncc(C)c2C2CCN(c3cc(N4CC(N5CCN(C(=O)C=C)CC5)C4C)nc(C(F)F)n3)C(C)C2)CC1 ; C=CC(=O)N1CCN(C2CN(c3cc(N4CCC(c5c(C)cnn5C5(OC)CC5)CC4)nc(C(F)(F)F)n3)C2C)CC1]

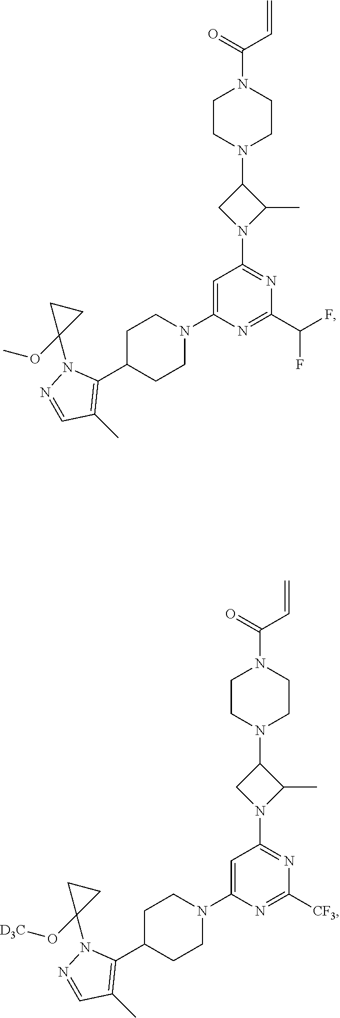 [SMILES: C=CC(=O)N1CCN(C2CN(c3cc(N4CCC(c5c(C)cnn5C5(OC)CC5)CC4)nc(C(F)F)n3)C2C)CC1 ; [2H]C([2H])([2H])OC1(n2ncc(C)c2C2CCN(c3cc(N4CC(N5CCN(C(=O)C=C)CC5)C4C)nc(C(F)(F)F)n3)CC2)CC1]

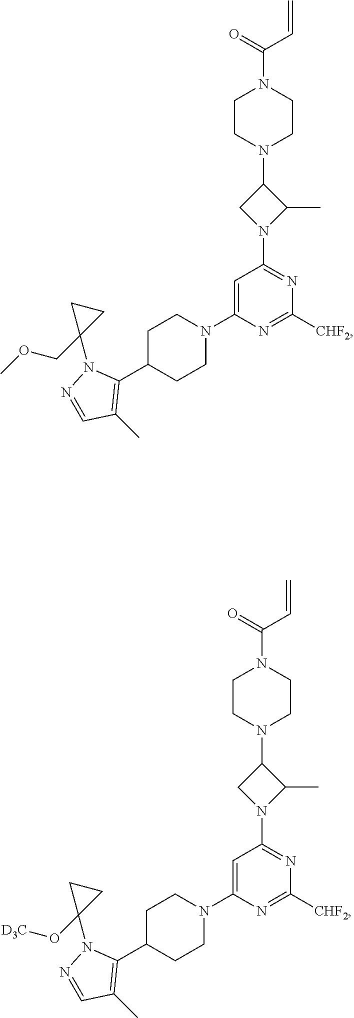 [SMILES: C=CC(=O)N1CCN(C2CN(c3cc(N4CCC(c5c(C)cnn5C5(COC)CC5)CC4)nc(C(F)F)n3)C2C)CC1 ; [2H]C([2H])([2H])OC1(n2ncc(C)c2C2CCN(c3cc(N4CC(N5CCN(C(=O)C=C)CC5)C4C)nc(C(F)F)n3)CC2)CC1]

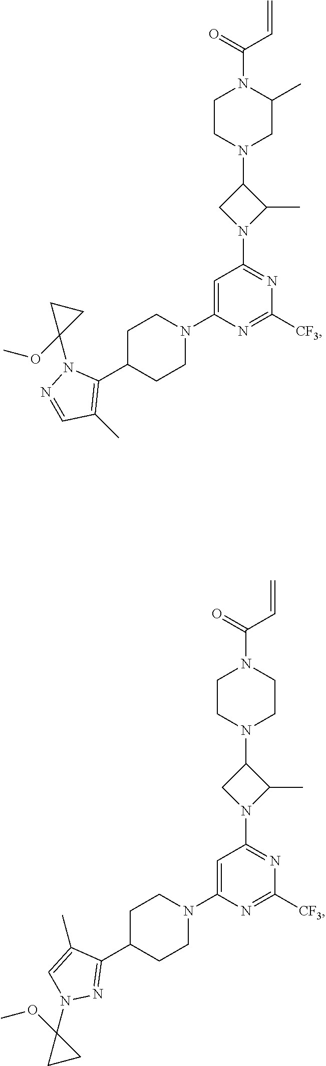 [SMILES: C=CC(=O)N1CCN(C2CN(c3cc(N4CCC(c5c(C)cnn5C5(OC)CC5)CC4)nc(C(F)(F)F)n3)C2C)CC1C ; C=CC(=O)N1CCN(C2CN(c3cc(N4CCC(c5nn(C6(OC)CC6)cc5C)CC4)nc(C(F)(F)F)n3)C2C)CC1]

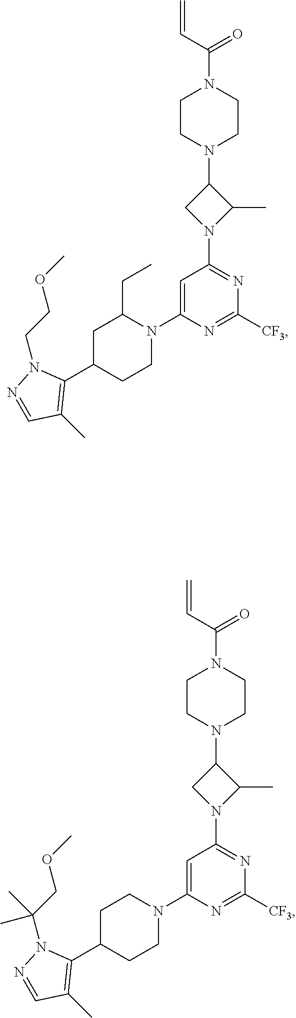 [SMILES: C=CC(=O)N1CCN(C2CN(c3cc(N4CCC(c5c(C)cnn5CCOC)CC4CC)nc(C(F)(F)F)n3)C2C)CC1 ; C=CC(=O)N1CCN(C2CN(c3cc(N4CCC(c5c(C)cnn5C(C)(C)COC)CC4)nc(C(F)(F)F)n3)C2C)CC1]

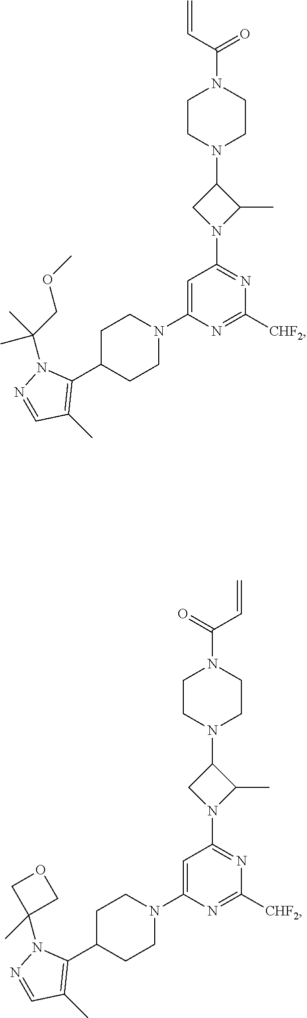 [SMILES: C=CC(=O)N1CCN(C2CN(c3cc(N4CCC(c5c(C)cnn5C(C)(C)COC)CC4)nc(C(F)F)n3)C2C)CC1 ; C=CC(=O)N1CCN(C2CN(c3cc(N4CCC(c5c(C)cnn5C5(C)COC5)CC4)nc(C(F)F)n3)C2C)CC1]

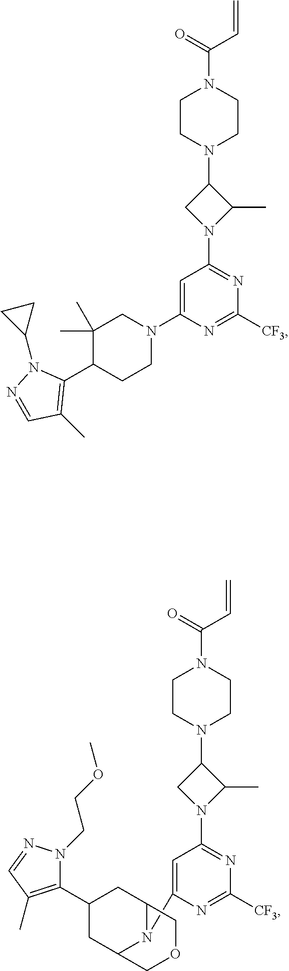 [SMILES: C=CC(=O)N1CCN(C2CN(c3cc(N4CCC(c5c(C)cnn5C5CC5)C(C)(C)C4)nc(C(F)(F)F)n3)C2C)CC1 ; C=CC(=O)N1CCN(C2CN(c3cc(N4C5COCC4CC(c4c(C)cnn4CCOC)C5)nc(C(F)(F)F)n3)C2C)CC1]

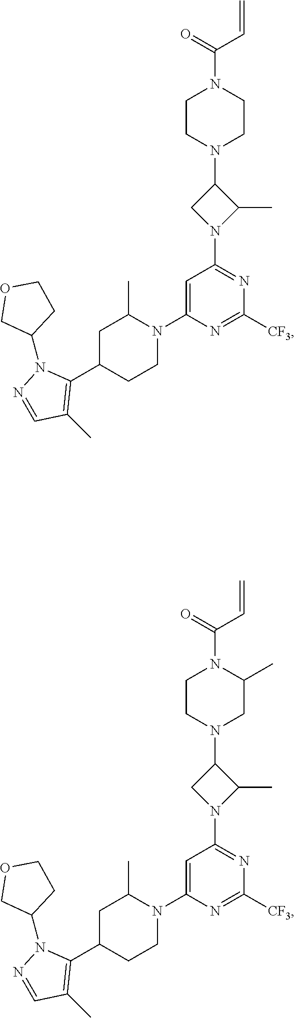 [SMILES: C=CC(=O)N1CCN(C2CN(c3cc(N4CCC(c5c(C)cnn5C5CCOC5)CC4C)nc(C(F)(F)F)n3)C2C)CC1 ; C=CC(=O)N1CCN(C2CN(c3cc(N4CCC(c5c(C)cnn5C5CCOC5)CC4C)nc(C(F)(F)F)n3)C2C)CC1C]

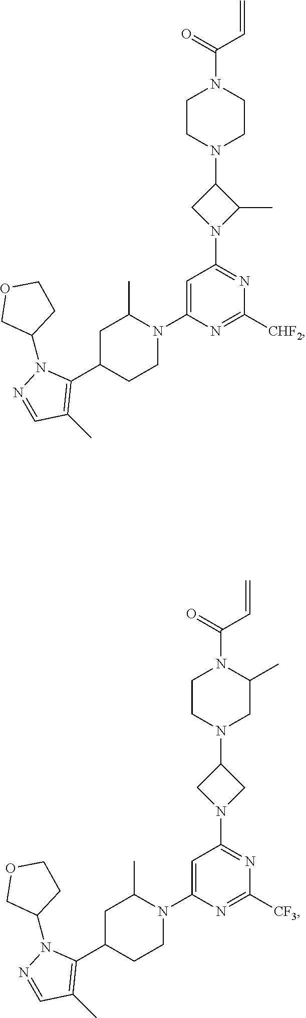 [SMILES: C=CC(=O)N1CCN(C2CN(c3cc(N4CCC(c5c(C)cnn5C5CCOC5)CC4C)nc(C(F)F)n3)C2C)CC1 ; C=CC(=O)N1CCN(C2CN(c3cc(N4CCC(c5c(C)cnn5C5CCOC5)CC4C)nc(C(F)(F)F)n3)C2)CC1C]

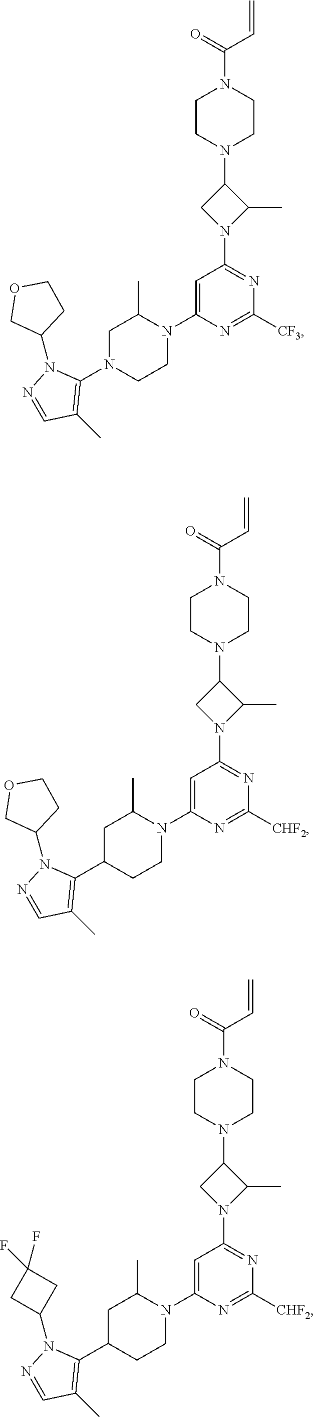 [SMILES: C=CC(=O)N1CCN(C2CN(c3cc(N4CCN(c5c(C)cnn5C5CCOC5)CC4C)nc(C(F)(F)F)n3)C2C)CC1 ; C=CC(=O)N1CCN(C2CN(c3cc(N4CCC(c5c(C)cnn5C5CCOC5)CC4C)nc(C(F)F)n3)C2C)CC1 ; C=CC(=O)N1CCN(C2CN(c3cc(N4CCC(c5c(C)cnn5C5CC(F)(F)C5)CC4C)nc(C(F)F)n3)C2C)CC1]

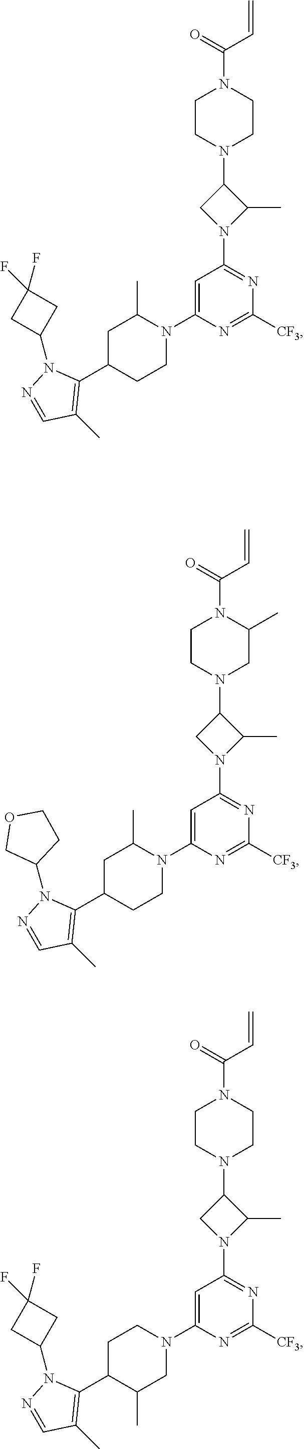 [SMILES: C=CC(=O)N1CCN(C2CN(c3cc(N4CCC(c5c(C)cnn5C5CC(F)(F)C5)CC4C)nc(C(F)(F)F)n3)C2C)CC1 ; C=CC(=O)N1CCN(C2CN(c3cc(N4CCC(c5c(C)cnn5C5CCOC5)CC4C)nc(C(F)(F)F)n3)C2C)CC1C ; C=CC(=O)N1CCN(C2CN(c3cc(N4CCC(c5c(C)cnn5C5CC(F)(F)C5)C(C)C4)nc(C(F)(F)F)n3)C2C)CC1]

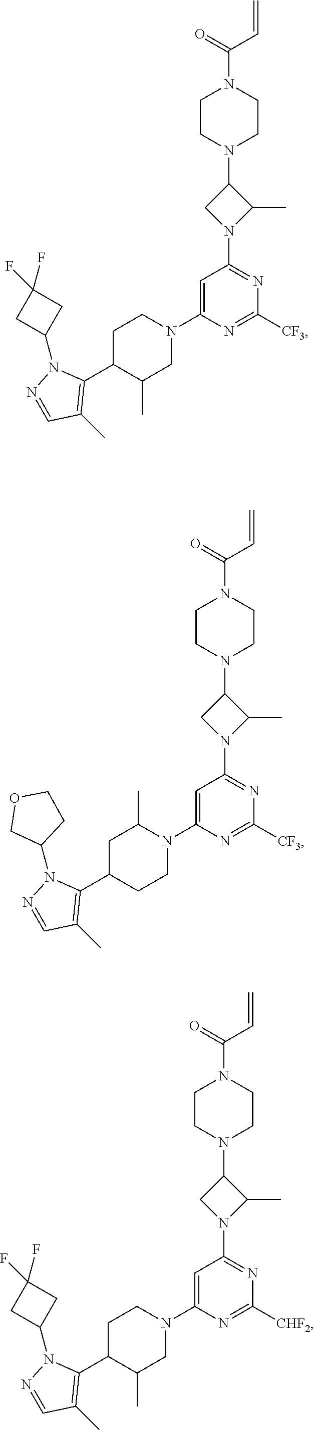 [SMILES: C=CC(=O)N1CCN(C2CN(c3cc(N4CCC(c5c(C)cnn5C5CC(F)(F)C5)C(C)C4)nc(C(F)(F)F)n3)C2C)CC1 ; C=CC(=O)N1CCN(C2CN(c3cc(N4CCC(c5c(C)cnn5C5CCOC5)CC4C)nc(C(F)(F)F)n3)C2C)CC1 ; C=CC(=O)N1CCN(C2CN(c3cc(N4CCC(c5c(C)cnn5C5CC(F)(F)C5)C(C)C4)nc(C(F)F)n3)C2C)CC1]

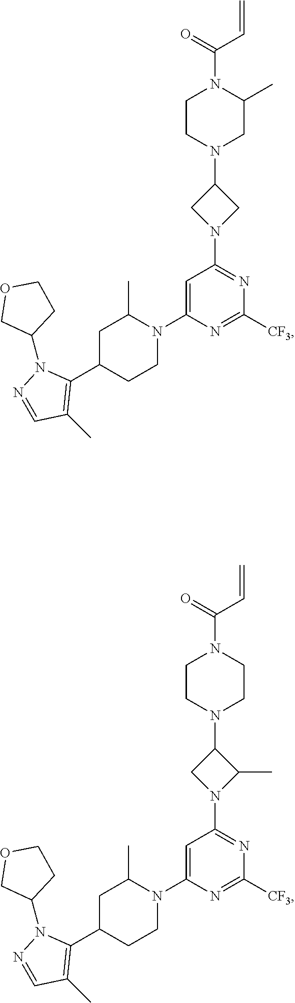 [SMILES: C=CC(=O)N1CCN(C2CN(c3cc(N4CCC(c5c(C)cnn5C5CCOC5)CC4C)nc(C(F)(F)F)n3)C2)CC1C ; C=CC(=O)N1CCN(C2CN(c3cc(N4CCC(c5c(C)cnn5C5CCOC5)CC4C)nc(C(F)(F)F)n3)C2C)CC1]

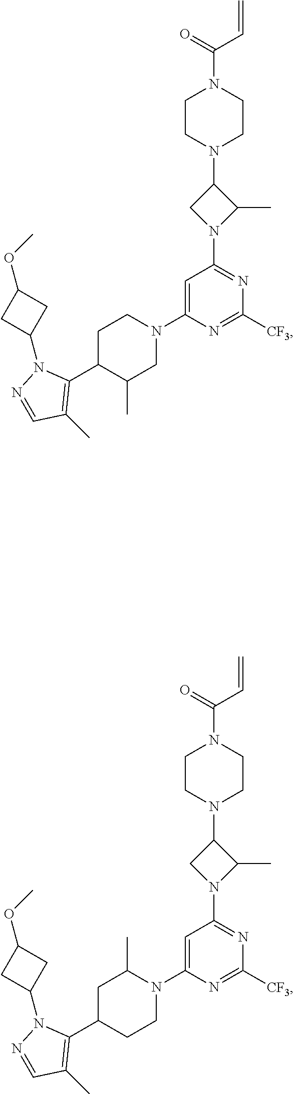 [SMILES: C=CC(=O)N1CCN(C2CN(c3cc(N4CCC(c5c(C)cnn5C5CC(OC)C5)C(C)C4)nc(C(F)(F)F)n3)C2C)CC1 ; C=CC(=O)N1CCN(C2CN(c3cc(N4CCC(c5c(C)cnn5C5CC(OC)C5)CC4C)nc(C(F)(F)F)n3)C2C)CC1]

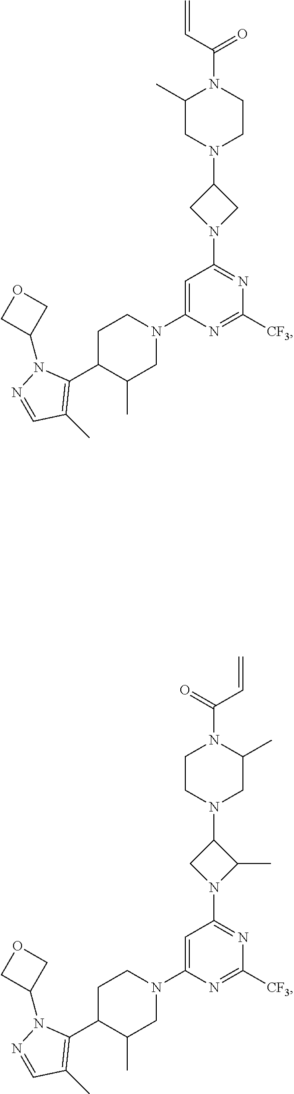 [SMILES: C=CC(=O)N1CCN(C2CN(c3cc(N4CCC(c5c(C)cnn5C5COC5)C(C)C4)nc(C(F)(F)F)n3)C2)CC1C ; C=CC(=O)N1CCN(C2CN(c3cc(N4CCC(c5c(C)cnn5C5COC5)C(C)C4)nc(C(F)(F)F)n3)C2C)CC1C]

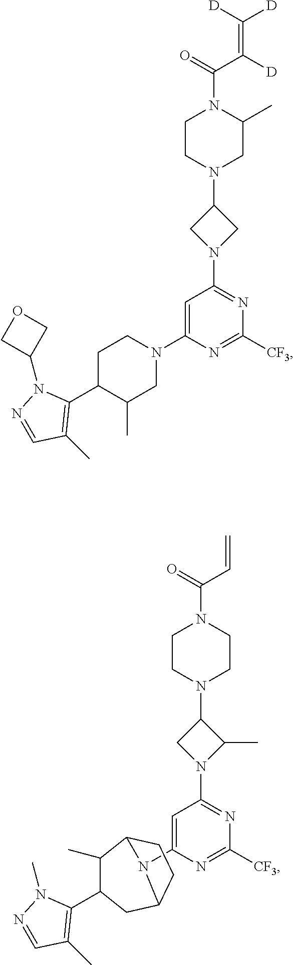 [SMILES: [2H]C([2H])=C([2H])C(=O)N1CCN(C2CN(c3cc(N4CCC(c5c(C)cnn5C5COC5)C(C)C4)nc(C(F)(F)F)n3)C2)CC1C ; C=CC(=O)N1CCN(C2CN(c3cc(N4C5CCC4C(C)C(c4c(C)cnn4C)C5)nc(C(F)(F)F)n3)C2C)CC1]

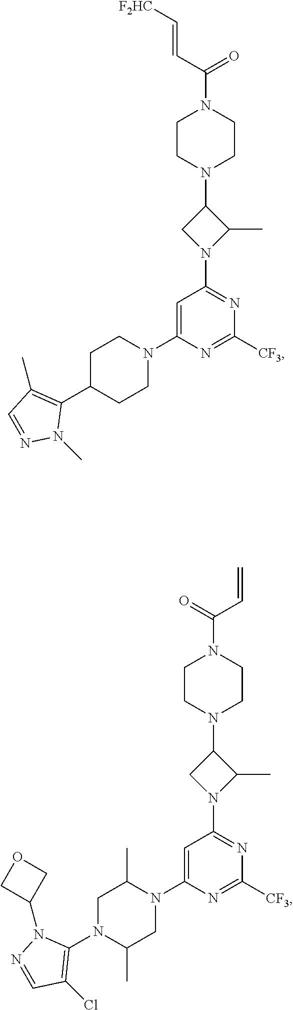 [SMILES: Cc1cnn(C)c1C1CCN(c2cc(N3CC(N4CCN(C(=O)/C=C/C(F)F)CC4)C3C)nc(C(F)(F)F)n2)CC1 ; C=CC(=O)N1CCN(C2CN(c3cc(N4CC(C)N(c5c(Cl)cnn5C5COC5)CC4C)nc(C(F)(F)F)n3)C2C)CC1]

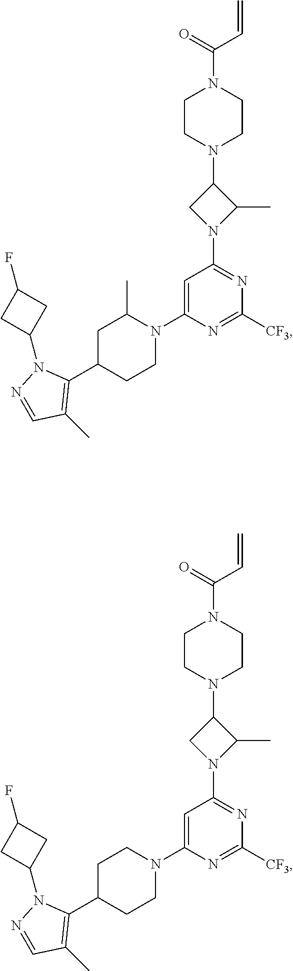 [SMILES: C=CC(=O)N1CCN(C2CN(c3cc(N4CCC(c5c(C)cnn5C5CC(F)C5)CC4C)nc(C(F)(F)F)n3)C2C)CC1 ; C=CC(=O)N1CCN(C2CN(c3cc(N4CCC(c5c(C)cnn5C5CC(F)C5)CC4)nc(C(F)(F)F)n3)C2C)CC1]

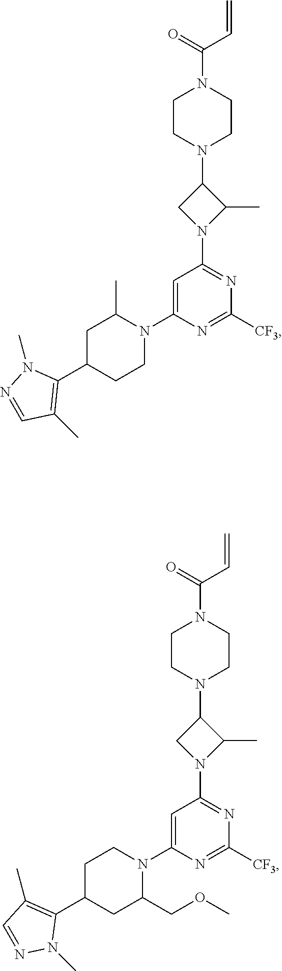 [SMILES: C=CC(=O)N1CCN(C2CN(c3cc(N4CCC(c5c(C)cnn5C)CC4C)nc(C(F)(F)F)n3)C2C)CC1 ; C=CC(=O)N1CCN(C2CN(c3cc(N4CCC(c5c(C)cnn5C)CC4COC)nc(C(F)(F)F)n3)C2C)CC1]

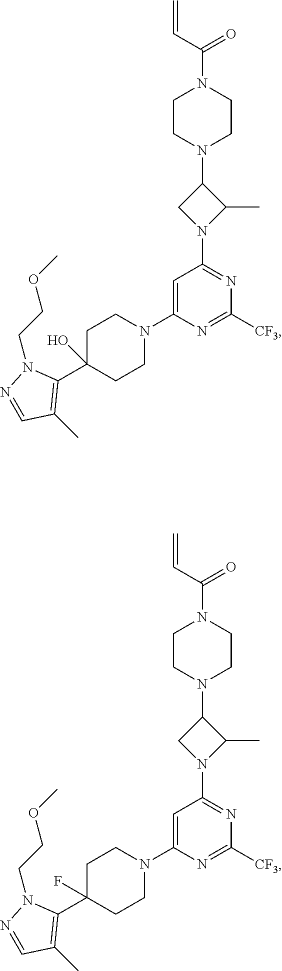 [SMILES: C=CC(=O)N1CCN(C2CN(c3cc(N4CCC(O)(c5c(C)cnn5CCOC)CC4)nc(C(F)(F)F)n3)C2C)CC1 ; C=CC(=O)N1CCN(C2CN(c3cc(N4CCC(F)(c5c(C)cnn5CCOC)CC4)nc(C(F)(F)F)n3)C2C)CC1]

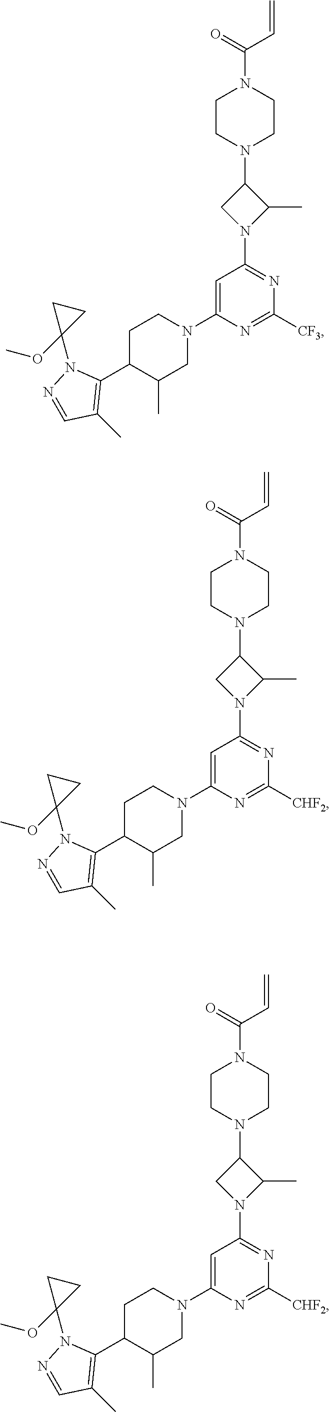 [SMILES: C=CC(=O)N1CCN(C2CN(c3cc(N4CCC(c5c(C)cnn5C5(OC)CC5)C(C)C4)nc(C(F)(F)F)n3)C2C)CC1 ; C=CC(=O)N1CCN(C2CN(c3cc(N4CCC(c5c(C)cnn5C5(OC)CC5)C(C)C4)nc(C(F)F)n3)C2C)CC1 ; C=CC(=O)N1CCN(C2CN(c3cc(N4CCC(c5c(C)cnn5C5(OC)CC5)C(C)C4)nc(C(F)F)n3)C2C)CC1]

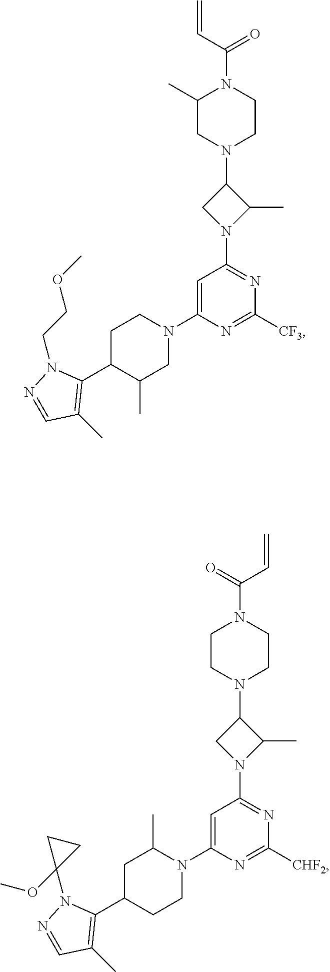 [SMILES: C=CC(=O)N1CCN(C2CN(c3cc(N4CCC(c5c(C)cnn5CCOC)C(C)C4)nc(C(F)(F)F)n3)C2C)CC1C ; C=CC(=O)N1CCN(C2CN(c3cc(N4CCC(c5c(C)cnn5C5(OC)CC5)CC4C)nc(C(F)F)n3)C2C)CC1]

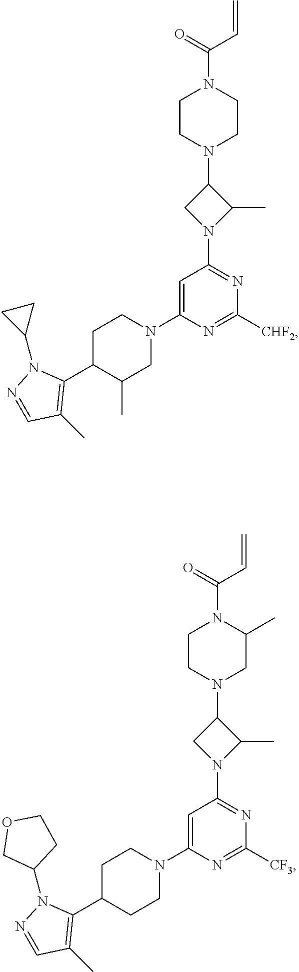 [SMILES: C=CC(=O)N1CCN(C2CN(c3cc(N4CCC(c5c(C)cnn5C5CC5)C(C)C4)nc(C(F)F)n3)C2C)CC1 ; C=CC(=O)N1CCN(C2CN(c3cc(N4CCC(c5c(C)cnn5C5CCOC5)CC4)nc(C(F)(F)F)n3)C2C)CC1C]

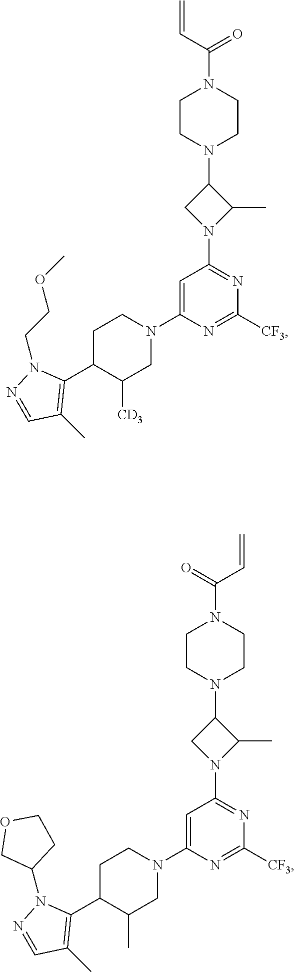 [SMILES: [2H]C([2H])([2H])C1CN(c2cc(N3CC(N4CCN(C(=O)C=C)CC4)C3C)nc(C(F)(F)F)n2)CCC1c1c(C)cnn1CCOC ; C=CC(=O)N1CCN(C2CN(c3cc(N4CCC(c5c(C)cnn5C5CCOC5)C(C)C4)nc(C(F)(F)F)n3)C2C)CC1]

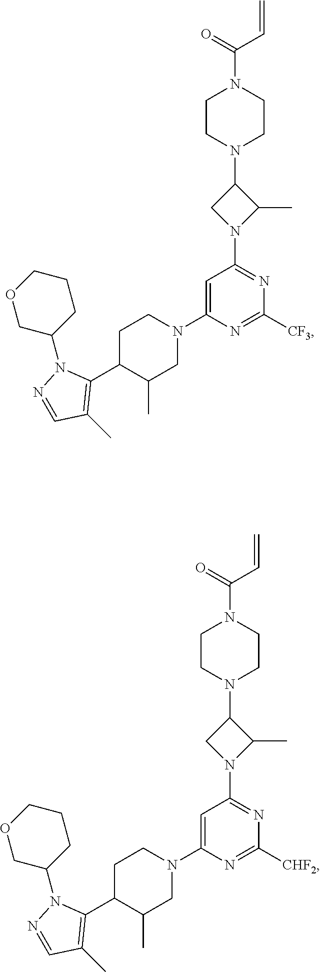 [SMILES: C=CC(=O)N1CCN(C2CN(c3cc(N4CCC(c5c(C)cnn5C5CCCOC5)C(C)C4)nc(C(F)(F)F)n3)C2C)CC1 ; C=CC(=O)N1CCN(C2CN(c3cc(N4CCC(c5c(C)cnn5C5CCCOC5)C(C)C4)nc(C(F)F)n3)C2C)CC1]

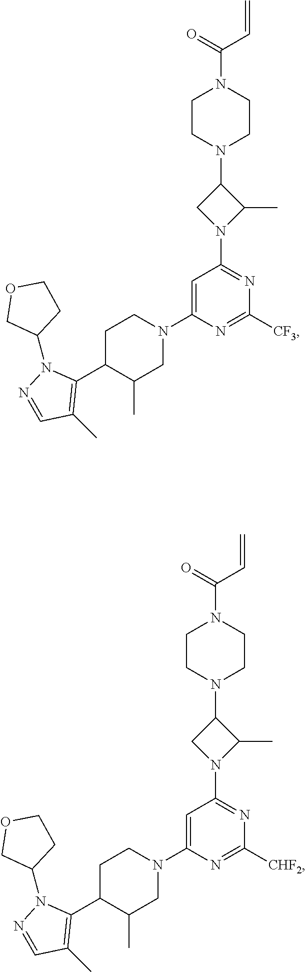 [SMILES: C=CC(=O)N1CCN(C2CN(c3cc(N4CCC(c5c(C)cnn5C5CCOC5)C(C)C4)nc(C(F)(F)F)n3)C2C)CC1 ; C=CC(=O)N1CCN(C2CN(c3cc(N4CCC(c5c(C)cnn5C5CCOC5)C(C)C4)nc(C(F)F)n3)C2C)CC1]

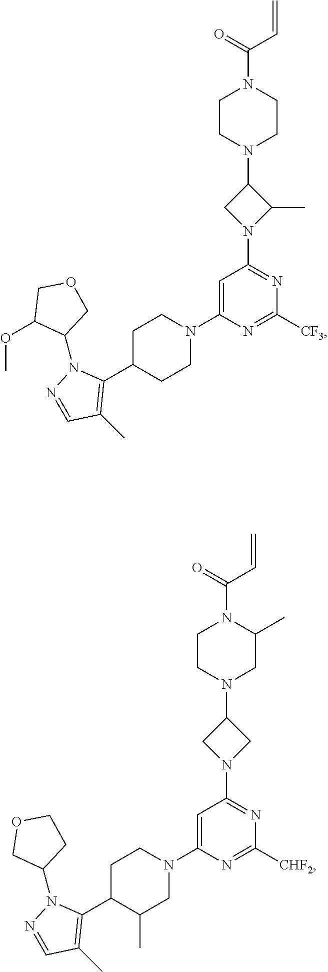 [SMILES: C=CC(=O)N1CCN(C2CN(c3cc(N4CCC(c5c(C)cnn5C5COCC5OC)CC4)nc(C(F)(F)F)n3)C2C)CC1 ; C=CC(=O)N1CCN(C2CN(c3cc(N4CCC(c5c(C)cnn5C5CCOC5)C(C)C4)nc(C(F)F)n3)C2)CC1C]

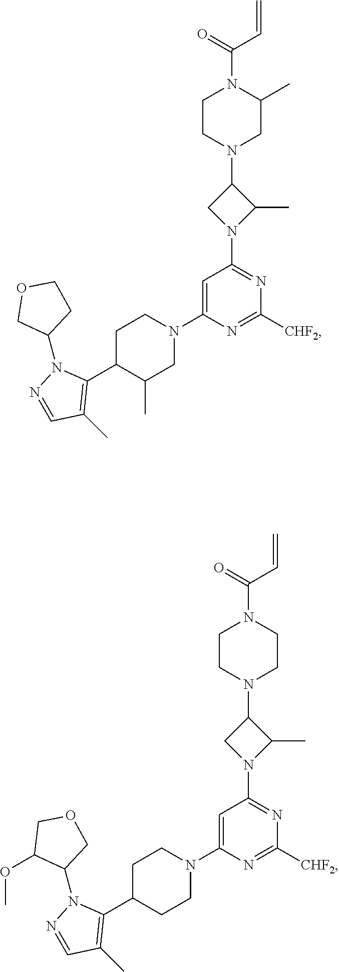 [SMILES: C=CC(=O)N1CCN(C2CN(c3cc(N4CCC(c5c(C)cnn5C5CCOC5)C(C)C4)nc(C(F)F)n3)C2C)CC1C ; C=CC(=O)N1CCN(C2CN(c3cc(N4CCC(c5c(C)cnn5C5COCC5OC)CC4)nc(C(F)F)n3)C2C)CC1]

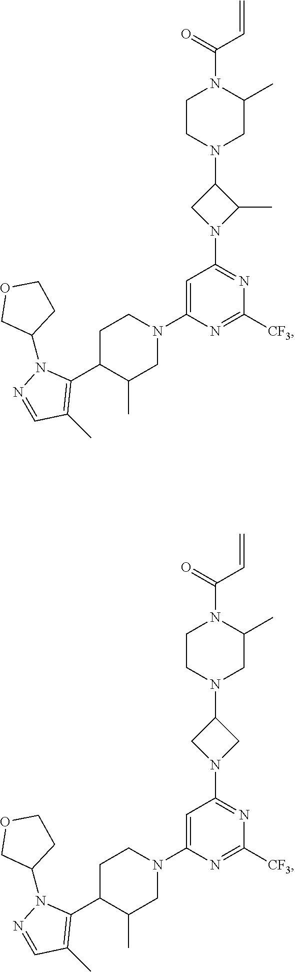 [SMILES: C=CC(=O)N1CCN(C2CN(c3cc(N4CCC(c5c(C)cnn5C5CCOC5)C(C)C4)nc(C(F)(F)F)n3)C2C)CC1C ; C=CC(=O)N1CCN(C2CN(c3cc(N4CCC(c5c(C)cnn5C5CCOC5)C(C)C4)nc(C(F)(F)F)n3)C2)CC1C]

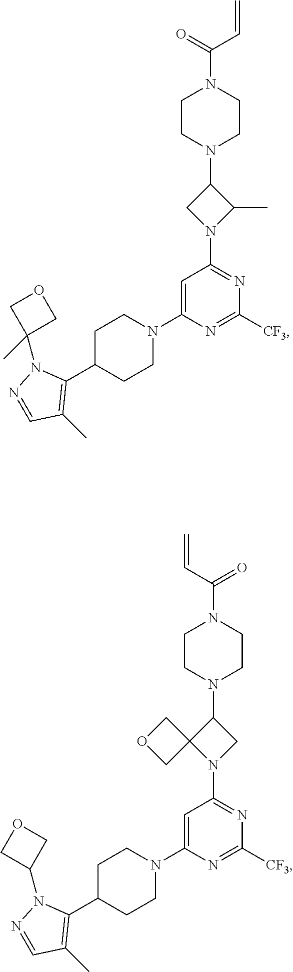 [SMILES: C=CC(=O)N1CCN(C2CN(c3cc(N4CCC(c5c(C)cnn5C5(C)COC5)CC4)nc(C(F)(F)F)n3)C2C)CC1 ; C=CC(=O)N1CCN(C2CN(c3cc(N4CCC(c5c(C)cnn5C5COC5)CC4)nc(C(F)(F)F)n3)C23COC3)CC1]

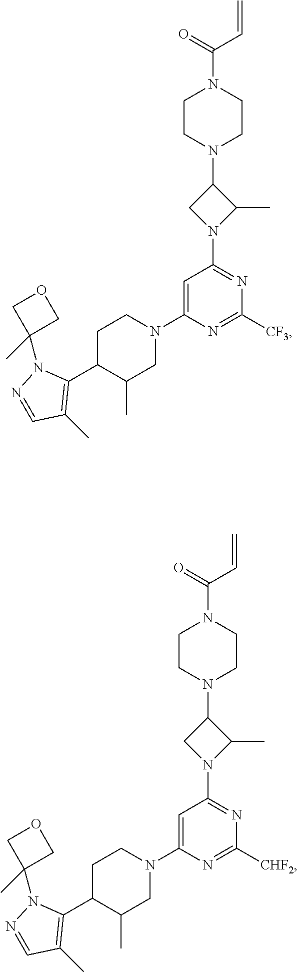 [SMILES: C=CC(=O)N1CCN(C2CN(c3cc(N4CCC(c5c(C)cnn5C5(C)COC5)C(C)C4)nc(C(F)(F)F)n3)C2C)CC1 ; C=CC(=O)N1CCN(C2CN(c3cc(N4CCC(c5c(C)cnn5C5(C)COC5)C(C)C4)nc(C(F)F)n3)C2C)CC1]

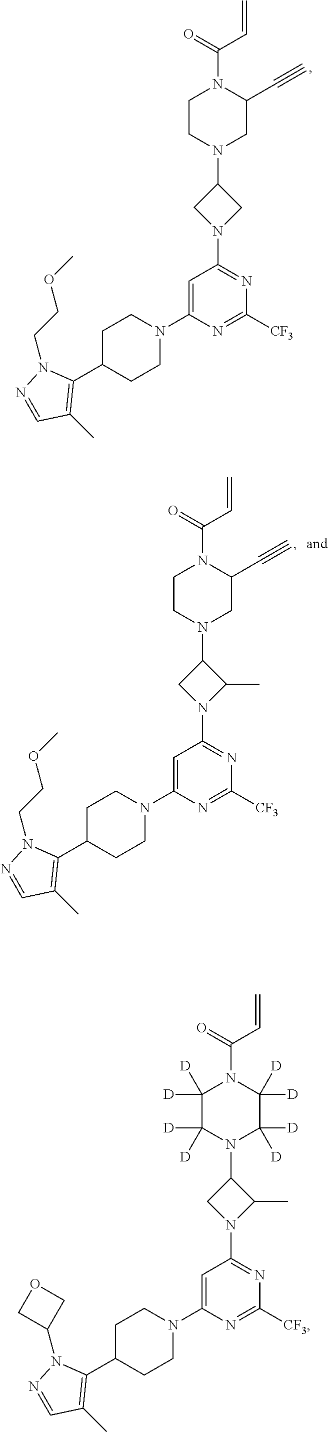 [SMILES: C#CC1CN(C2CN(c3cc(N4CCC(c5c(C)cnn5CCOC)CC4)nc(C(F)(F)F)n3)C2)CCN1C(=O)C=C ; C#CC1CN(C2CN(c3cc(N4CCC(c5c(C)cnn5CCOC)CC4)nc(C(F)(F)F)n3)C2C)CCN1C(=O)C=C ; [2H]C1([2H])N(C(=O)C=C)C([2H])([2H])C([2H])([2H])N(C2CN(c3cc(N4CCC(c5c(C)cnn5C5COC5)CC4)nc(C(F)(F)F)n3)C2C)C1([2H])[2H]]

and pharmaceutically acceptable salts thereof.

In some cases, the disclosure provides compounds of Formula (I) wherein A is N; X is

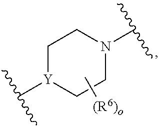

and Z is thiazolyl, isothiazolyl, oxazolyl, isoxazolyl, imidazolyl, or triazolyl, wherein each of the foregoing is optionally substituted. Examples of such compounds include, but are not limited to:

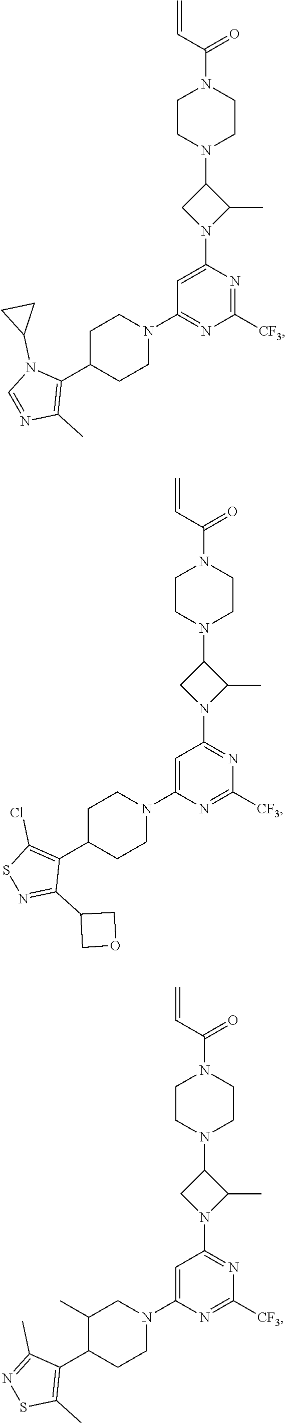 [SMILES: C=CC(=O)N1CCN(C2CN(c3cc(N4CCC(c5c(C)ncn5C5CC5)CC4)nc(C(F)(F)F)n3)C2C)CC1 ; C=CC(=O)N1CCN(C2CN(c3cc(N4CCC(c5c(C6COC6)nsc5Cl)CC4)nc(C(F)(F)F)n3)C2C)CC1 ; C=CC(=O)N1CCN(C2CN(c3cc(N4CCC(c5c(C)nsc5C)C(C)C4)nc(C(F)(F)F)n3)C2C)CC1]

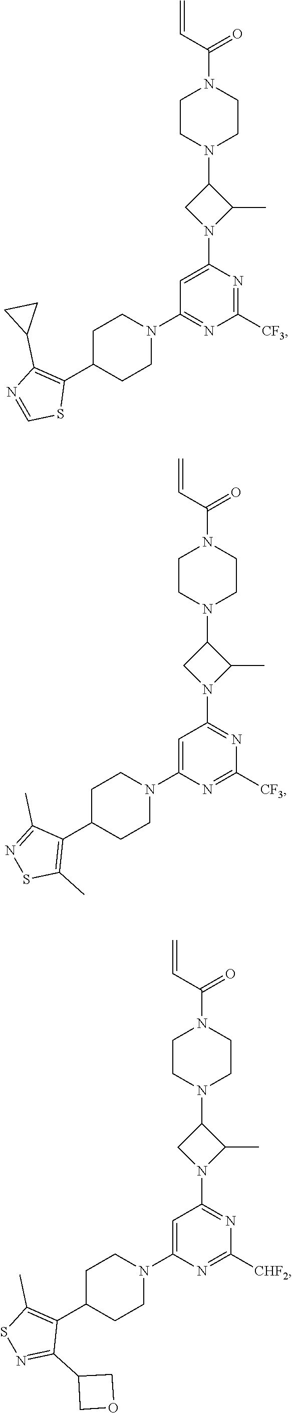 [SMILES: C=CC(=O)N1CCN(C2CN(c3cc(N4CCC(c5scnc5C5CC5)CC4)nc(C(F)(F)F)n3)C2C)CC1 ; C=CC(=O)N1CCN(C2CN(c3cc(N4CCC(c5c(C)nsc5C)CC4)nc(C(F)(F)F)n3)C2C)CC1 ; C=CC(=O)N1CCN(C2CN(c3cc(N4CCC(c5c(C6COC6)nsc5C)CC4)nc(C(F)F)n3)C2C)CC1]

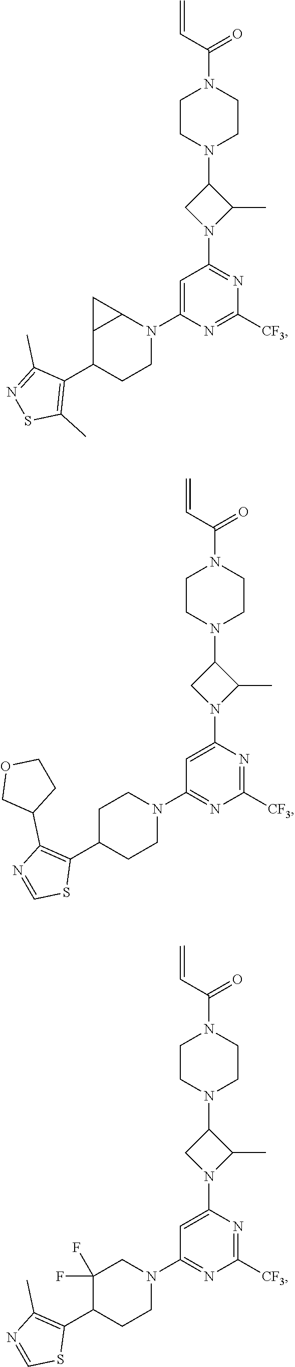 [SMILES: C=CC(=O)N1CCN(C2CN(c3cc(N4CCC(c5c(C)nsc5C)C5CC54)nc(C(F)(F)F)n3)C2C)CC1 ; C=CC(=O)N1CCN(C2CN(c3cc(N4CCC(c5scnc5C5CCOC5)CC4)nc(C(F)(F)F)n3)C2C)CC1 ; C=CC(=O)N1CCN(C2CN(c3cc(N4CCC(c5scnc5C)C(F)(F)C4)nc(C(F)(F)F)n3)C2C)CC1]

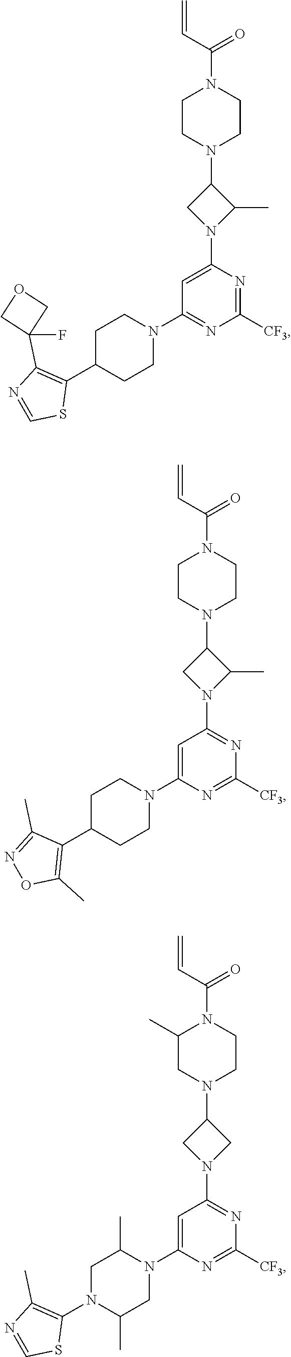 [SMILES: C=CC(=O)N1CCN(C2CN(c3cc(N4CCC(c5scnc5C5(F)COC5)CC4)nc(C(F)(F)F)n3)C2C)CC1 ; C=CC(=O)N1CCN(C2CN(c3cc(N4CCC(c5c(C)noc5C)CC4)nc(C(F)(F)F)n3)C2C)CC1 ; C=CC(=O)N1CCN(C2CN(c3cc(N4CC(C)N(c5scnc5C)CC4C)nc(C(F)(F)F)n3)C2)CC1C]

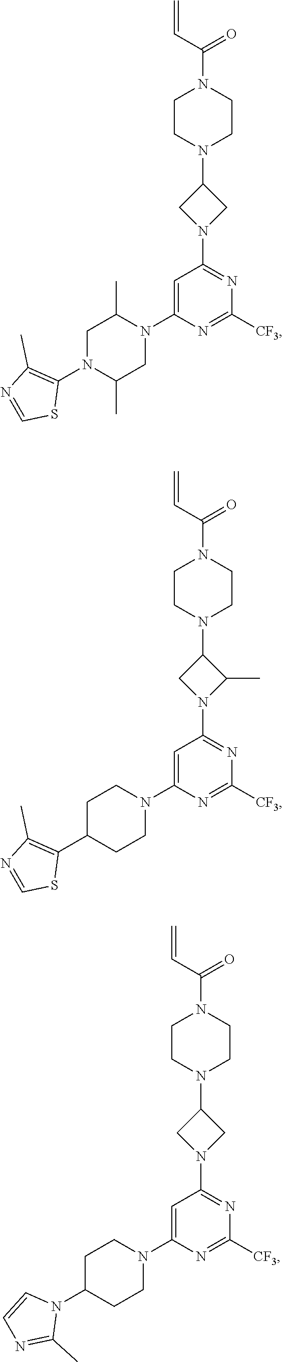 [SMILES: C=CC(=O)N1CCN(C2CN(c3cc(N4CC(C)N(c5scnc5C)CC4C)nc(C(F)(F)F)n3)C2)CC1 ; C=CC(=O)N1CCN(C2CN(c3cc(N4CCC(c5scnc5C)CC4)nc(C(F)(F)F)n3)C2C)CC1 ; C=CC(=O)N1CCN(C2CN(c3cc(N4CCC(n5ccnc5C)CC4)nc(C(F)(F)F)n3)C2)CC1]

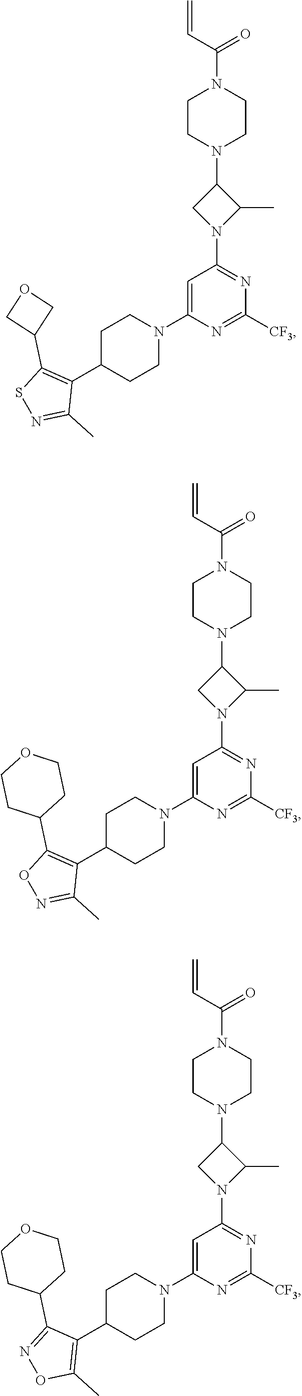 [SMILES: C=CC(=O)N1CCN(C2CN(c3cc(N4CCC(c5c(C)nsc5C5COC5)CC4)nc(C(F)(F)F)n3)C2C)CC1 ; C=CC(=O)N1CCN(C2CN(c3cc(N4CCC(c5c(C)noc5C5CCOCC5)CC4)nc(C(F)(F)F)n3)C2C)CC1 ; C=CC(=O)N1CCN(C2CN(c3cc(N4CCC(c5c(C6CCOCC6)noc5C)CC4)nc(C(F)(F)F)n3)C2C)CC1]

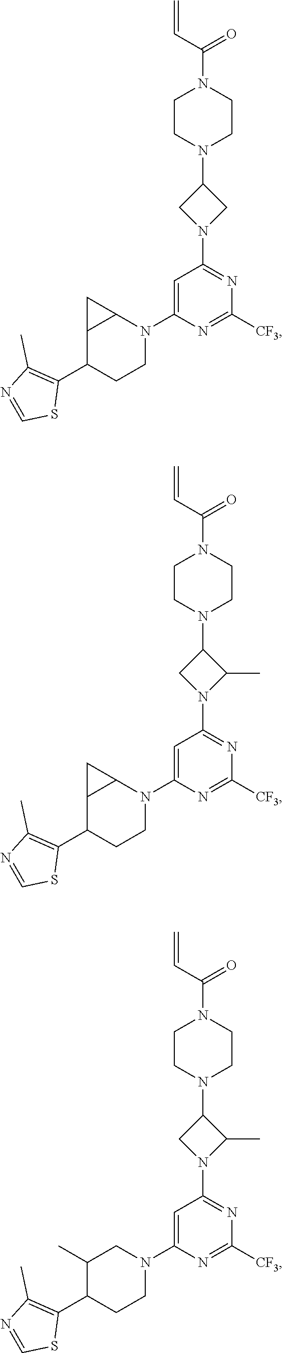 [SMILES: C=CC(=O)N1CCN(C2CN(c3cc(N4CCC(c5scnc5C)C5CC54)nc(C(F)(F)F)n3)C2)CC1 ; C=CC(=O)N1CCN(C2CN(c3cc(N4CCC(c5scnc5C)C5CC54)nc(C(F)(F)F)n3)C2C)CC1 ; C=CC(=O)N1CCN(C2CN(c3cc(N4CCC(c5scnc5C)C(C)C4)nc(C(F)(F)F)n3)C2C)CC1]

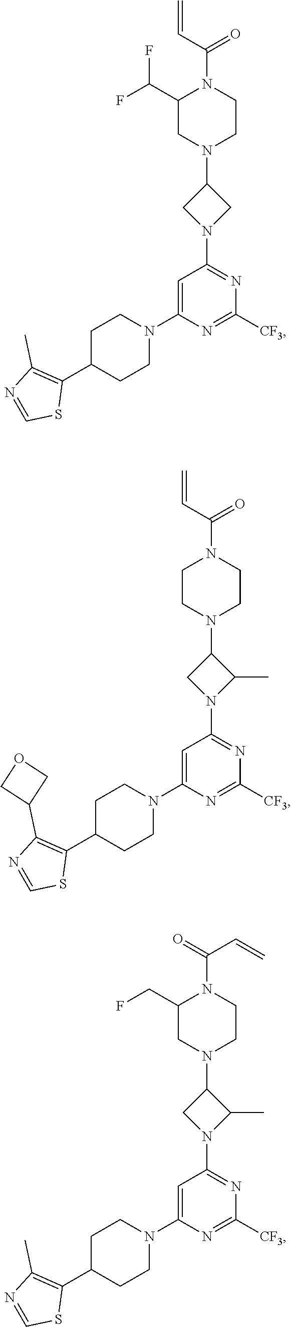 [SMILES: C=CC(=O)N1CCN(C2CN(c3cc(N4CCC(c5scnc5C)CC4)nc(C(F)(F)F)n3)C2)CC1C(F)F ; C=CC(=O)N1CCN(C2CN(c3cc(N4CCC(c5scnc5C5COC5)CC4)nc(C(F)(F)F)n3)C2C)CC1 ; C=CC(=O)N1CCN(C2CN(c3cc(N4CCC(c5scnc5C)CC4)nc(C(F)(F)F)n3)C2C)CC1CF]

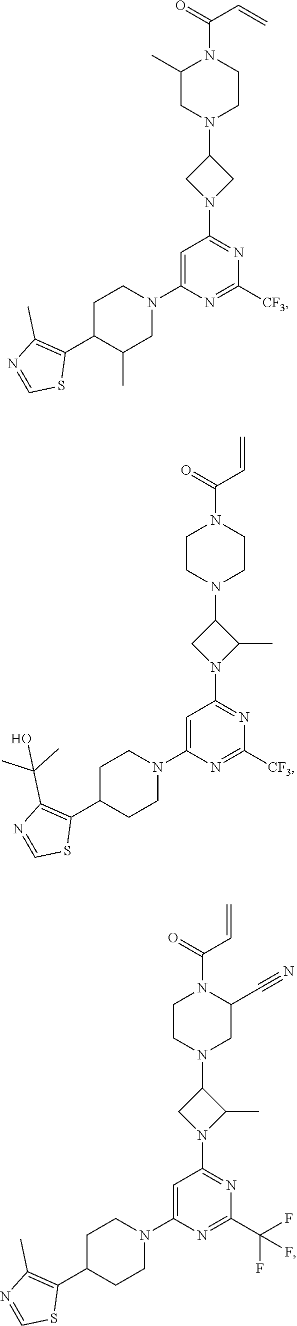 [SMILES: C=CC(=O)N1CCN(C2CN(c3cc(N4CCC(c5scnc5C)C(C)C4)nc(C(F)(F)F)n3)C2)CC1C ; C=CC(=O)N1CCN(C2CN(c3cc(N4CCC(c5scnc5C(C)(C)O)CC4)nc(C(F)(F)F)n3)C2C)CC1 ; C=CC(=O)N1CCN(C2CN(c3cc(N4CCC(c5scnc5C)CC4)nc(C(F)(F)F)n3)C2C)CC1C#N]

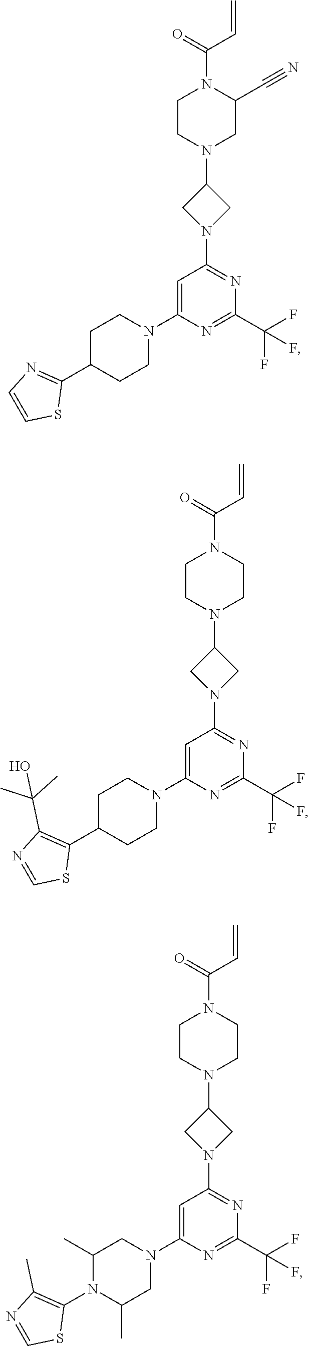 [SMILES: C=CC(=O)N1CCN(C2CN(c3cc(N4CCC(c5nccs5)CC4)nc(C(F)(F)F)n3)C2)CC1C#N ; C=CC(=O)N1CCN(C2CN(c3cc(N4CCC(c5scnc5C(C)(C)O)CC4)nc(C(F)(F)F)n3)C2)CC1 ; C=CC(=O)N1CCN(C2CN(c3cc(N4CC(C)N(c5scnc5C)C(C)C4)nc(C(F)(F)F)n3)C2)CC1]

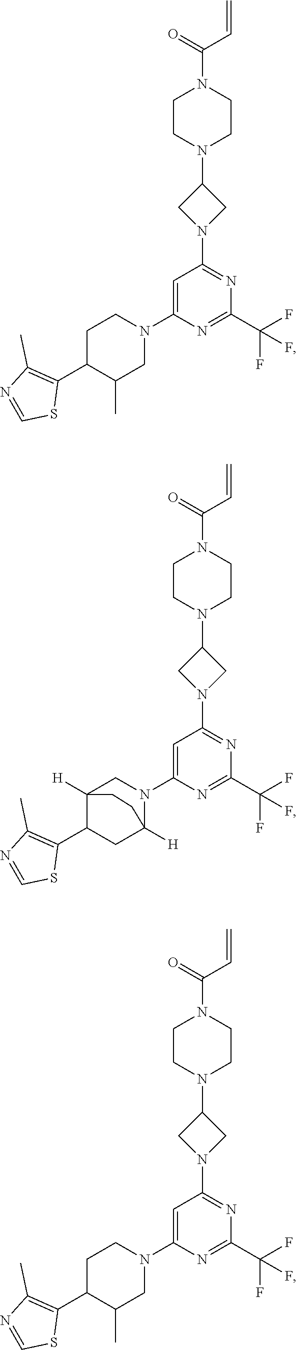 [SMILES: C=CC(=O)N1CCN(C2CN(c3cc(N4CCC(c5scnc5C)C(C)C4)nc(C(F)(F)F)n3)C2)CC1 ; C=CC(=O)N1CCN(C2CN(c3cc(N4CC5CCC4CC5c4scnc4C)nc(C(F)(F)F)n3)C2)CC1 ; C=CC(=O)N1CCN(C2CN(c3cc(N4CCC(c5scnc5C)C(C)C4)nc(C(F)(F)F)n3)C2)CC1]

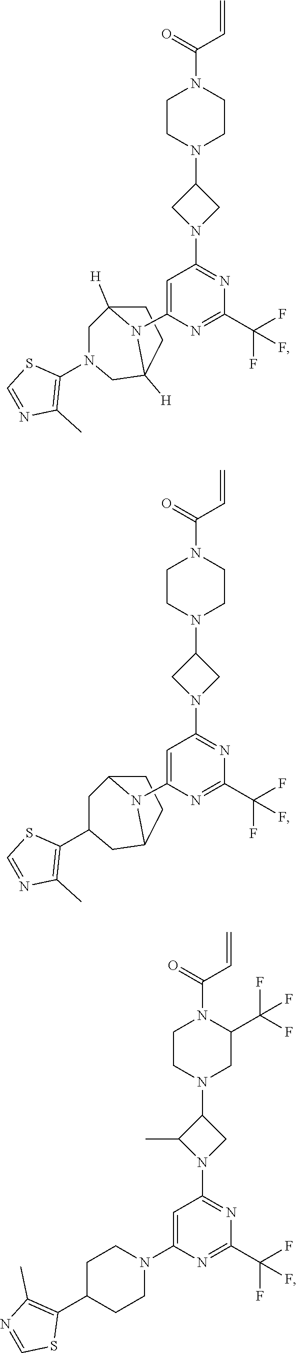 [SMILES: C=CC(=O)N1CCN(C2CN(c3cc(N4C5CCC4CN(c4scnc4C)C5)nc(C(F)(F)F)n3)C2)CC1 ; C=CC(=O)N1CCN(C2CN(c3cc(N4C5CCC4CC(c4scnc4C)C5)nc(C(F)(F)F)n3)C2)CC1 ; C=CC(=O)N1CCN(C2CN(c3cc(N4CCC(c5scnc5C)CC4)nc(C(F)(F)F)n3)C2C)CC1C(F)(F)F]

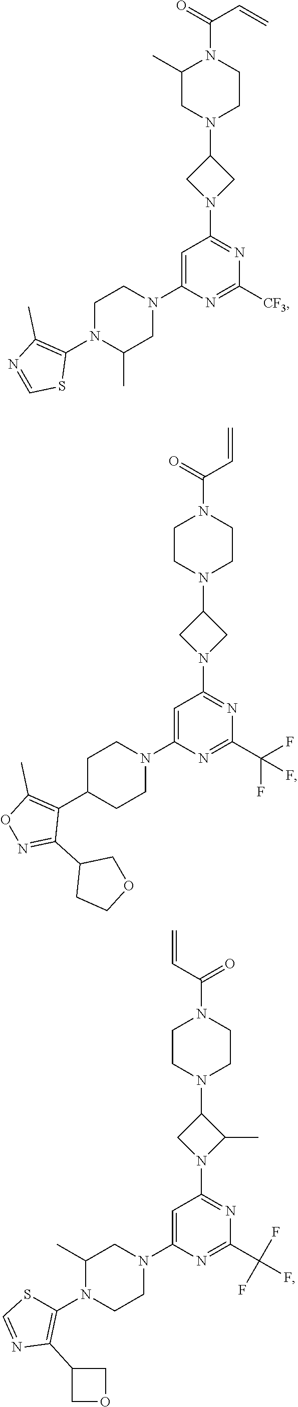 [SMILES: C=CC(=O)N1CCN(C2CN(c3cc(N4CCN(c5scnc5C)C(C)C4)nc(C(F)(F)F)n3)C2)CC1C ; C=CC(=O)N1CCN(C2CN(c3cc(N4CCC(c5c(C6CCOC6)noc5C)CC4)nc(C(F)(F)F)n3)C2)CC1 ; C=CC(=O)N1CCN(C2CN(c3cc(N4CCN(c5scnc5C5COC5)C(C)C4)nc(C(F)(F)F)n3)C2C)CC1]

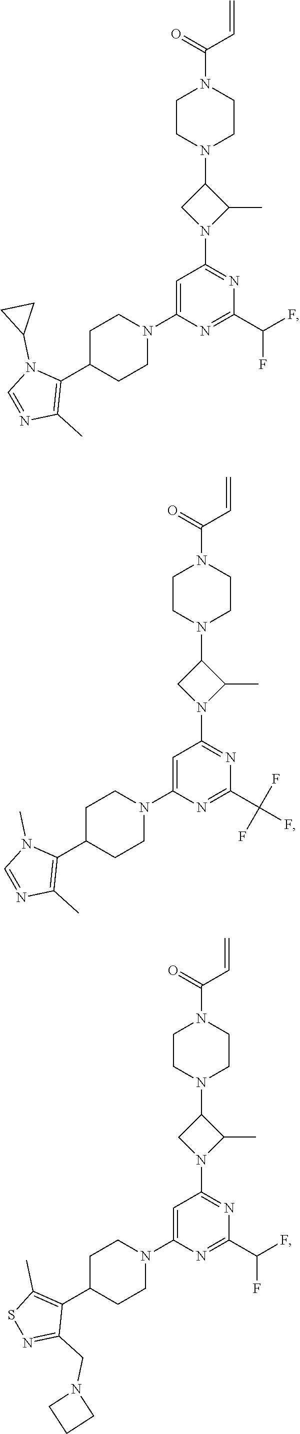 [SMILES: C=CC(=O)N1CCN(C2CN(c3cc(N4CCC(c5c(C)ncn5C5CC5)CC4)nc(C(F)F)n3)C2C)CC1 ; C=CC(=O)N1CCN(C2CN(c3cc(N4CCC(c5c(C)ncn5C)CC4)nc(C(F)(F)F)n3)C2C)CC1 ; C=CC(=O)N1CCN(C2CN(c3cc(N4CCC(c5c(CN6CCC6)nsc5C)CC4)nc(C(F)F)n3)C2C)CC1]

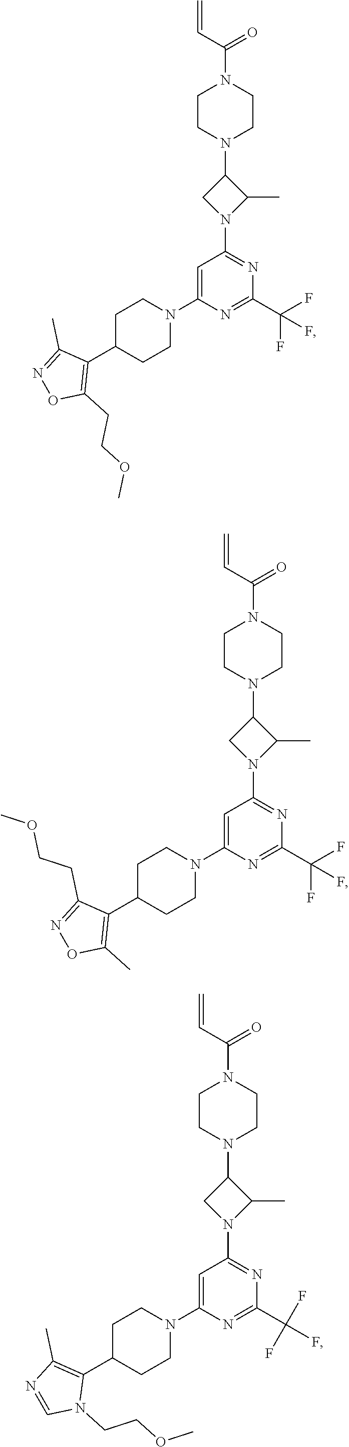 [SMILES: C=CC(=O)N1CCN(C2CN(c3cc(N4CCC(c5c(C)noc5CCOC)CC4)nc(C(F)(F)F)n3)C2C)CC1 ; C=CC(=O)N1CCN(C2CN(c3cc(N4CCC(c5c(CCOC)noc5C)CC4)nc(C(F)(F)F)n3)C2C)CC1 ; C=CC(=O)N1CCN(C2CN(c3cc(N4CCC(c5c(C)ncn5CCOC)CC4)nc(C(F)(F)F)n3)C2C)CC1]

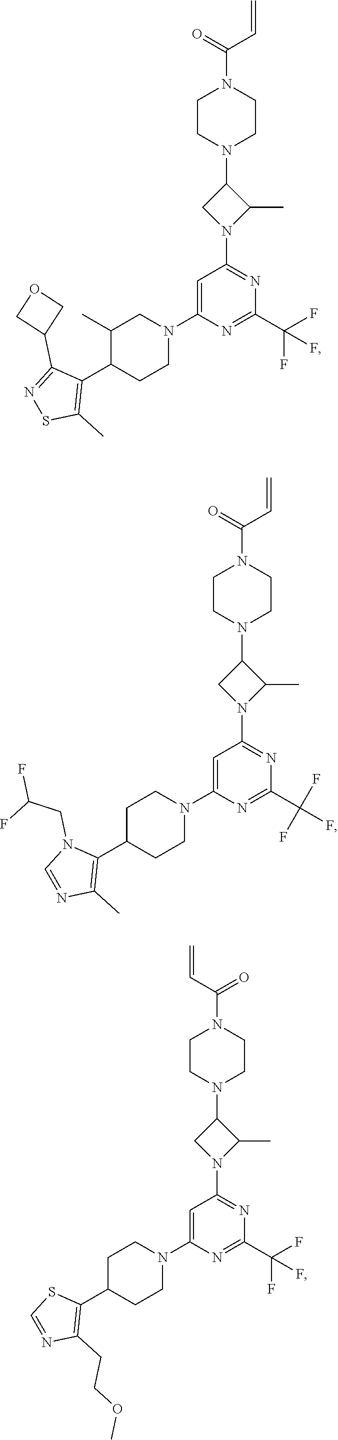 [SMILES: C=CC(=O)N1CCN(C2CN(c3cc(N4CCC(c5c(C6COC6)nsc5C)C(C)C4)nc(C(F)(F)F)n3)C2C)CC1 ; C=CC(=O)N1CCN(C2CN(c3cc(N4CCC(c5c(C)ncn5CC(F)F)CC4)nc(C(F)(F)F)n3)C2C)CC1 ; C=CC(=O)N1CCN(C2CN(c3cc(N4CCC(c5scnc5CCOC)CC4)nc(C(F)(F)F)n3)C2C)CC1]

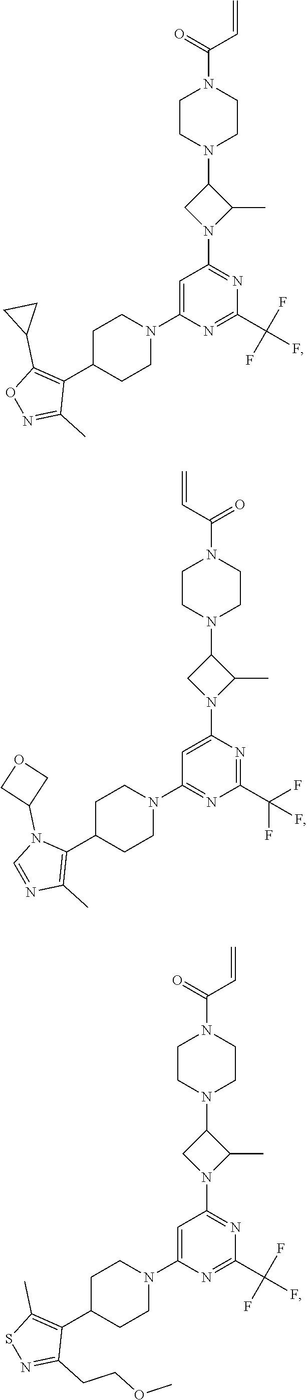 [SMILES: C=CC(=O)N1CCN(C2CN(c3cc(N4CCC(c5c(C)noc5C5CC5)CC4)nc(C(F)(F)F)n3)C2C)CC1 ; C=CC(=O)N1CCN(C2CN(c3cc(N4CCC(c5c(C)ncn5C5COC5)CC4)nc(C(F)(F)F)n3)C2C)CC1 ; C=CC(=O)N1CCN(C2CN(c3cc(N4CCC(c5c(CCOC)nsc5C)CC4)nc(C(F)(F)F)n3)C2C)CC1]

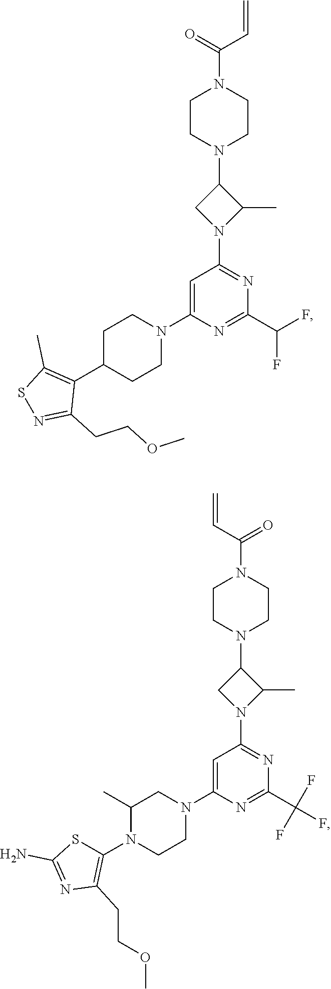 [SMILES: C=CC(=O)N1CCN(C2CN(c3cc(N4CCC(c5c(CCOC)nsc5C)CC4)nc(C(F)F)n3)C2C)CC1 ; C=CC(=O)N1CCN(C2CN(c3cc(N4CCN(c5sc(N)nc5CCOC)C(C)C4)nc(C(F)(F)F)n3)C2C)CC1]

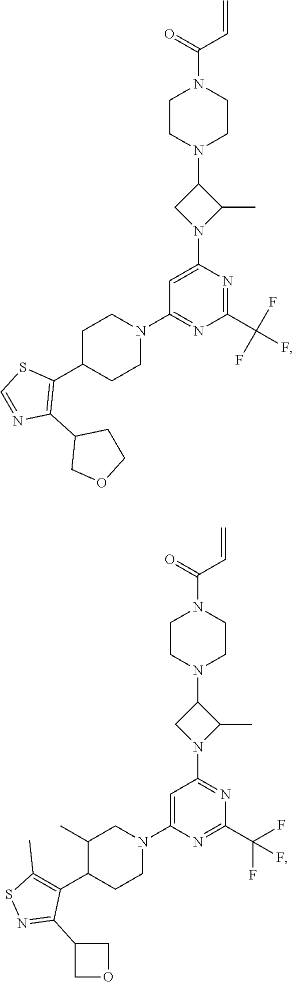 [SMILES: C=CC(=O)N1CCN(C2CN(c3cc(N4CCC(c5scnc5C5CCOC5)CC4)nc(C(F)(F)F)n3)C2C)CC1 ; C=CC(=O)N1CCN(C2CN(c3cc(N4CCC(c5c(C6COC6)nsc5C)C(C)C4)nc(C(F)(F)F)n3)C2C)CC1]

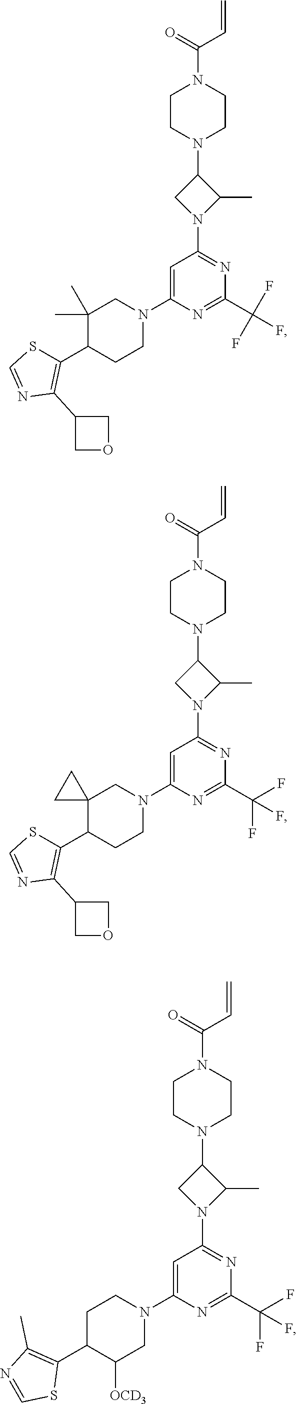

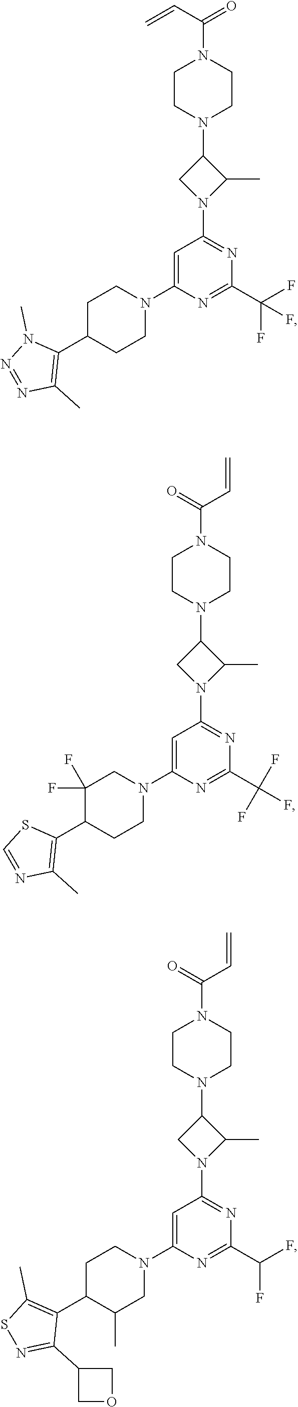 [SMILES: C=CC(=O)N1CCN(C2CN(c3cc(N4CCC(c5c(C)nnn5C)CC4)nc(C(F)(F)F)n3)C2C)CC1 ; C=CC(=O)N1CCN(C2CN(c3cc(N4CCC(c5scnc5C)C(F)(F)C4)nc(C(F)(F)F)n3)C2C)CC1 ; C=CC(=O)N1CCN(C2CN(c3cc(N4CCC(c5c(C6COC6)nsc5C)C(C)C4)nc(C(F)F)n3)C2C)CC1]

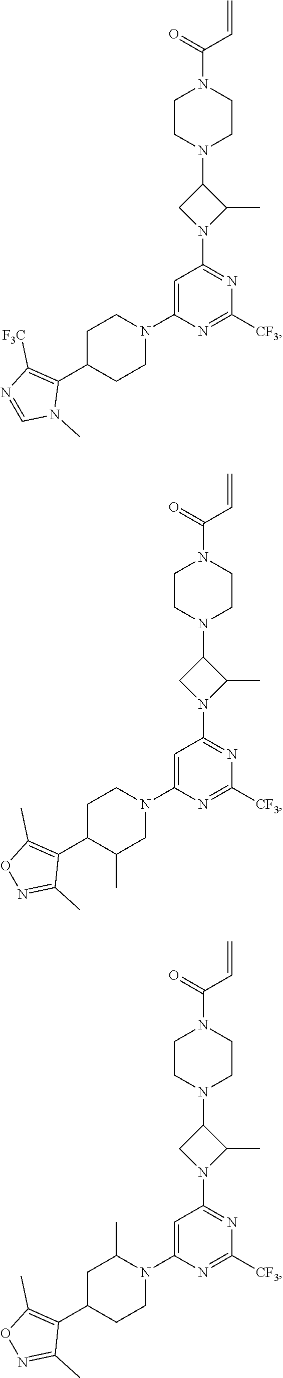 [SMILES: C=CC(=O)N1CCN(C2CN(c3cc(N4CCC(c5c(C(F)(F)F)ncn5C)CC4)nc(C(F)(F)F)n3)C2C)CC1 ; C=CC(=O)N1CCN(C2CN(c3cc(N4CCC(c5c(C)noc5C)C(C)C4)nc(C(F)(F)F)n3)C2C)CC1 ; C=CC(=O)N1CCN(C2CN(c3cc(N4CCC(c5c(C)noc5C)CC4C)nc(C(F)(F)F)n3)C2C)CC1]

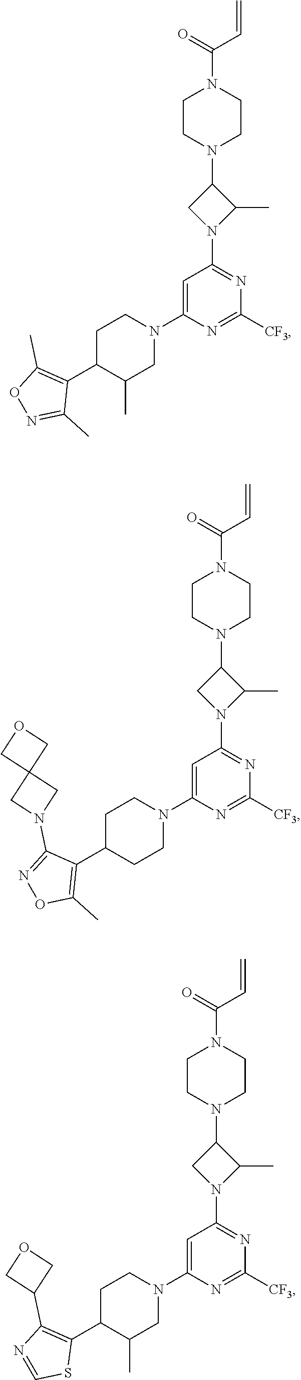 [SMILES: C=CC(=O)N1CCN(C2CN(c3cc(N4CCC(c5c(C)noc5C)C(C)C4)nc(C(F)(F)F)n3)C2C)CC1 ; C=CC(=O)N1CCN(C2CN(c3cc(N4CCC(c5c(N6CC7(COC7)C6)noc5C)CC4)nc(C(F)(F)F)n3)C2C)CC1 ; C=CC(=O)N1CCN(C2CN(c3cc(N4CCC(c5scnc5C5COC5)C(C)C4)nc(C(F)(F)F)n3)C2C)CC1]

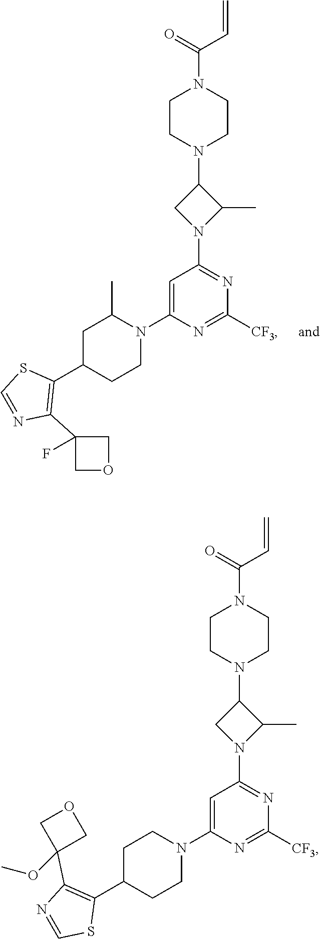 [SMILES: C=CC(=O)N1CCN(C2CN(c3cc(N4CCC(c5scnc5C5(F)COC5)CC4C)nc(C(F)(F)F)n3)C2C)CC1 ; C=CC(=O)N1CCN(C2CN(c3cc(N4CCC(c5scnc5C5(OC)COC5)CC4)nc(C(F)(F)F)n3)C2C)CC1]

and pharmaceutically acceptable salts thereof.

In some cases, the disclosure provides compounds of Formula (i) wherein A is N; X is

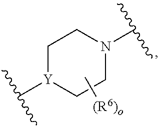

and Z is optionally substituted pyridyl. Examples of such compounds include but are not limited to:

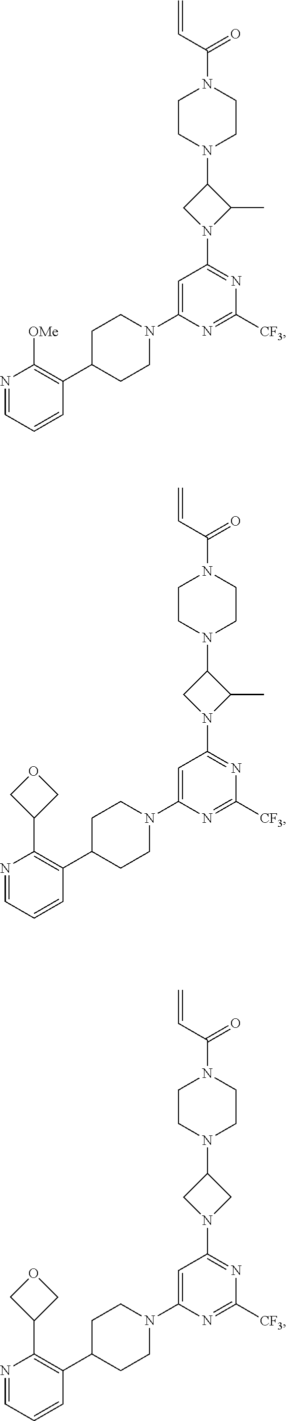 [SMILES: C=CC(=O)N1CCN(C2CN(c3cc(N4CCC(c5cccnc5OC)CC4)nc(C(F)(F)F)n3)C2C)CC1 ; C=CC(=O)N1CCN(C2CN(c3cc(N4CCC(c5cccnc5C5COC5)CC4)nc(C(F)(F)F)n3)C2C)CC1 ; C=CC(=O)N1CCN(C2CN(c3cc(N4CCC(c5cccnc5C5COC5)CC4)nc(C(F)(F)F)n3)C2)CC1]

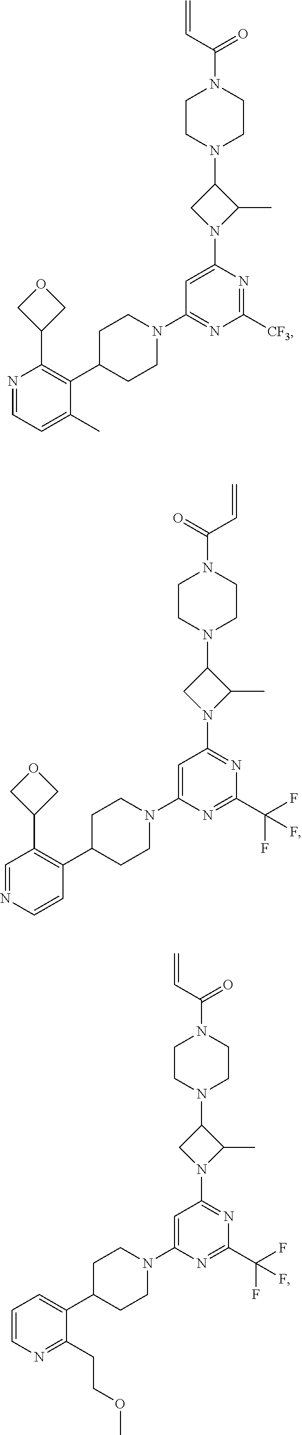 [SMILES: C=CC(=O)N1CCN(C2CN(c3cc(N4CCC(c5c(C)ccnc5C5COC5)CC4)nc(C(F)(F)F)n3)C2C)CC1 ; C=CC(=O)N1CCN(C2CN(c3cc(N4CCC(c5ccncc5C5COC5)CC4)nc(C(F)(F)F)n3)C2C)CC1 ; C=CC(=O)N1CCN(C2CN(c3cc(N4CCC(c5cccnc5CCOC)CC4)nc(C(F)(F)F)n3)C2C)CC1]

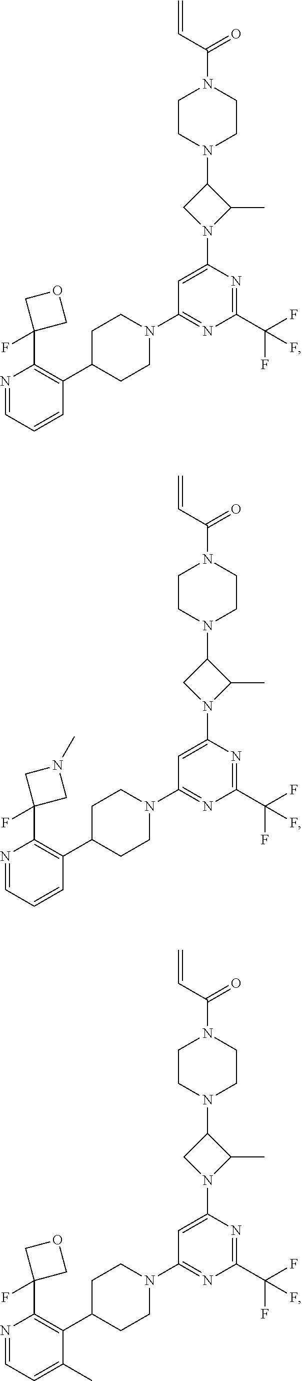 [SMILES: C=CC(=O)N1CCN(C2CN(c3cc(N4CCC(c5cccnc5C5(F)COC5)CC4)nc(C(F)(F)F)n3)C2C)CC1 ; C=CC(=O)N1CCN(C2CN(c3cc(N4CCC(c5cccnc5C5(F)CN(C)C5)CC4)nc(C(F)(F)F)n3)C2C)CC1 ; C=CC(=O)N1CCN(C2CN(c3cc(N4CCC(c5c(C)ccnc5C5(F)COC5)CC4)nc(C(F)(F)F)n3)C2C)CC1]

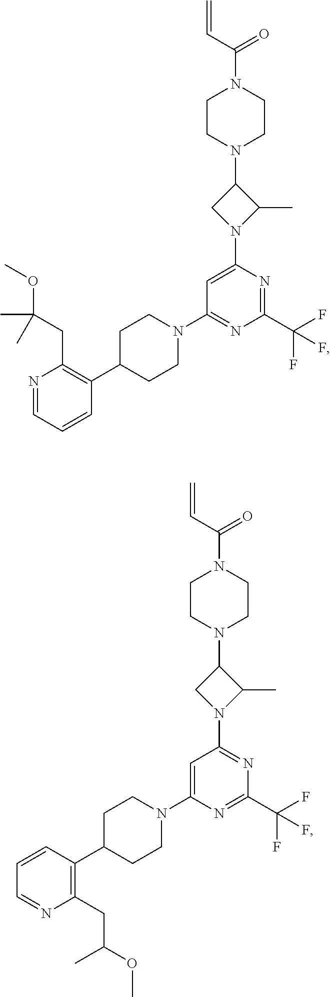 [SMILES: C=CC(=O)N1CCN(C2CN(c3cc(N4CCC(c5cccnc5CC(C)(C)OC)CC4)nc(C(F)(F)F)n3)C2C)CC1 ; C=CC(=O)N1CCN(C2CN(c3cc(N4CCC(c5cccnc5CC(C)OC)CC4)nc(C(F)(F)F)n3)C2C)CC1]

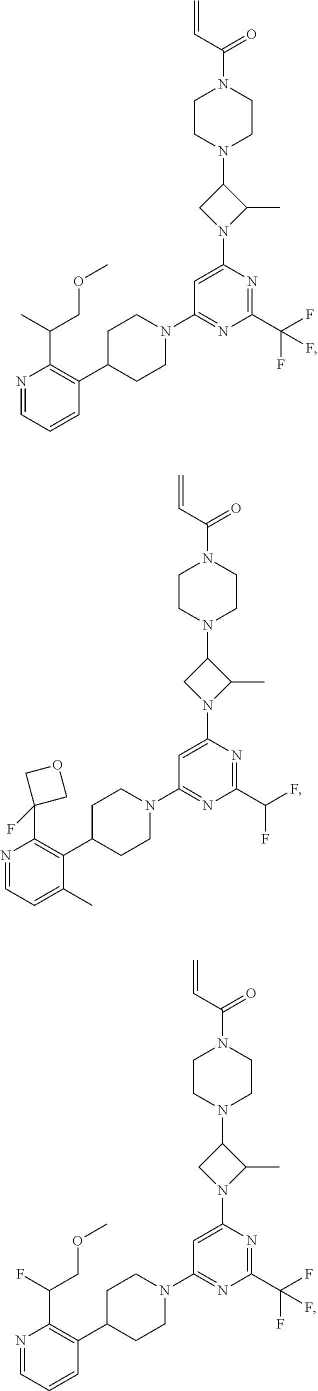 [SMILES: C=CC(=O)N1CCN(C2CN(c3cc(N4CCC(c5cccnc5C(C)COC)CC4)nc(C(F)(F)F)n3)C2C)CC1 ; C=CC(=O)N1CCN(C2CN(c3cc(N4CCC(c5c(C)ccnc5C5(F)COC5)CC4)nc(C(F)F)n3)C2C)CC1 ; C=CC(=O)N1CCN(C2CN(c3cc(N4CCC(c5cccnc5C(F)COC)CC4)nc(C(F)(F)F)n3)C2C)CC1]

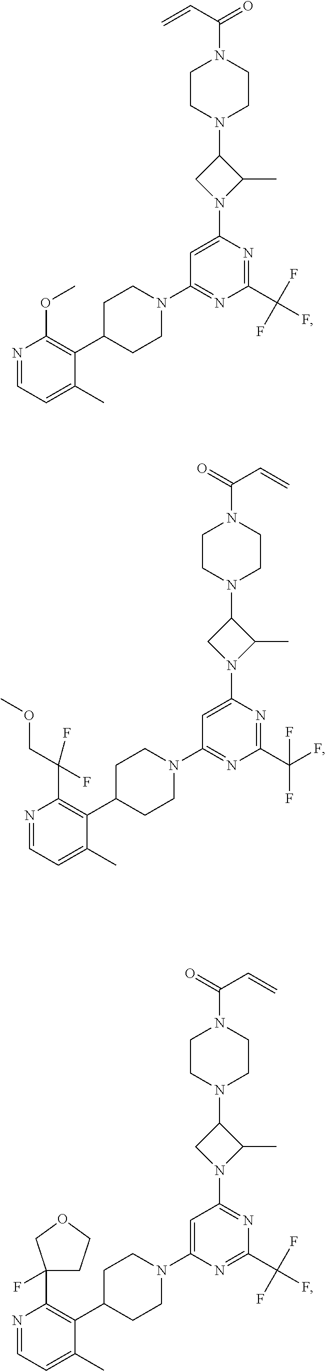 [SMILES: C=CC(=O)N1CCN(C2CN(c3cc(N4CCC(c5c(C)ccnc5OC)CC4)nc(C(F)(F)F)n3)C2C)CC1 ; C=CC(=O)N1CCN(C2CN(c3cc(N4CCC(c5c(C)ccnc5C(F)(F)COC)CC4)nc(C(F)(F)F)n3)C2C)CC1 ; C=CC(=O)N1CCN(C2CN(c3cc(N4CCC(c5c(C)ccnc5C5(F)CCOC5)CC4)nc(C(F)(F)F)n3)C2C)CC1]

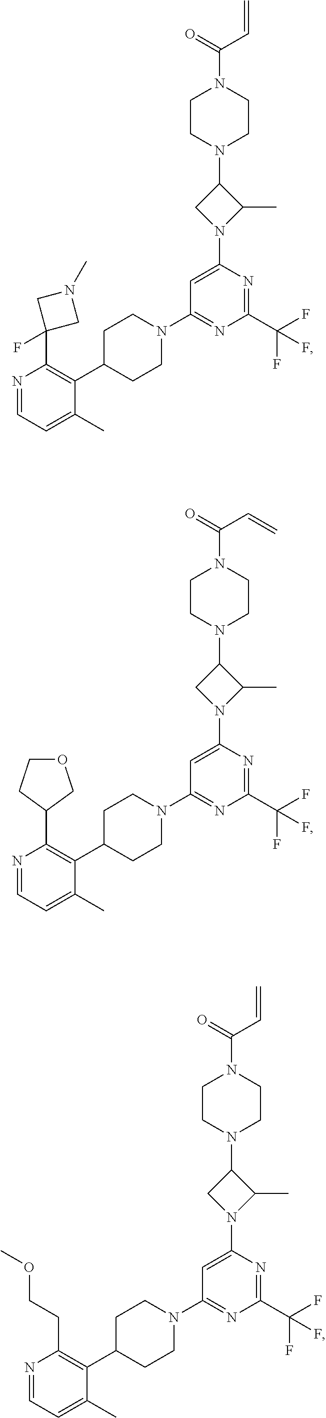 [SMILES: C=CC(=O)N1CCN(C2CN(c3cc(N4CCC(c5c(C)ccnc5C5(F)CN(C)C5)CC4)nc(C(F)(F)F)n3)C2C)CC1 ; C=CC(=O)N1CCN(C2CN(c3cc(N4CCC(c5c(C)ccnc5C5CCOC5)CC4)nc(C(F)(F)F)n3)C2C)CC1 ; C=CC(=O)N1CCN(C2CN(c3cc(N4CCC(c5c(C)ccnc5CCOC)CC4)nc(C(F)(F)F)n3)C2C)CC1]

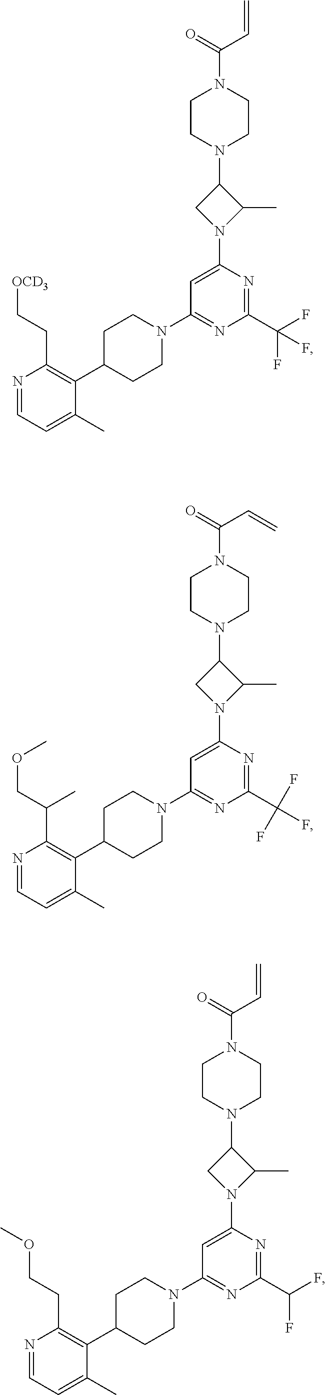

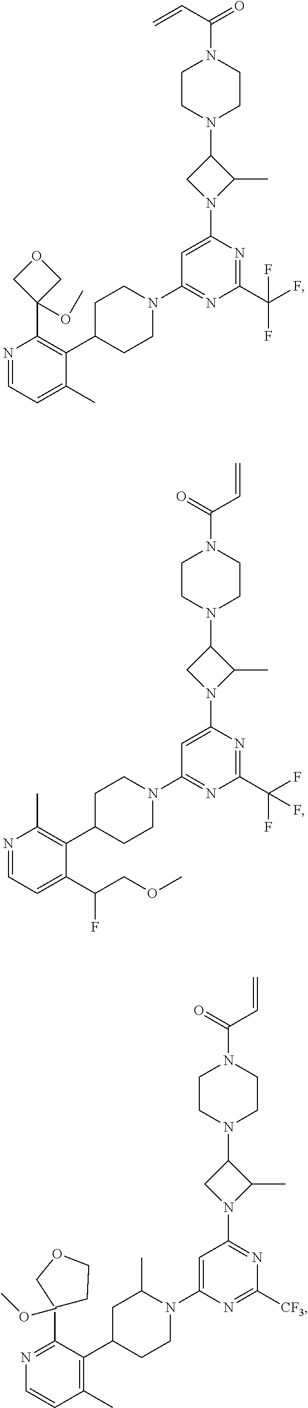

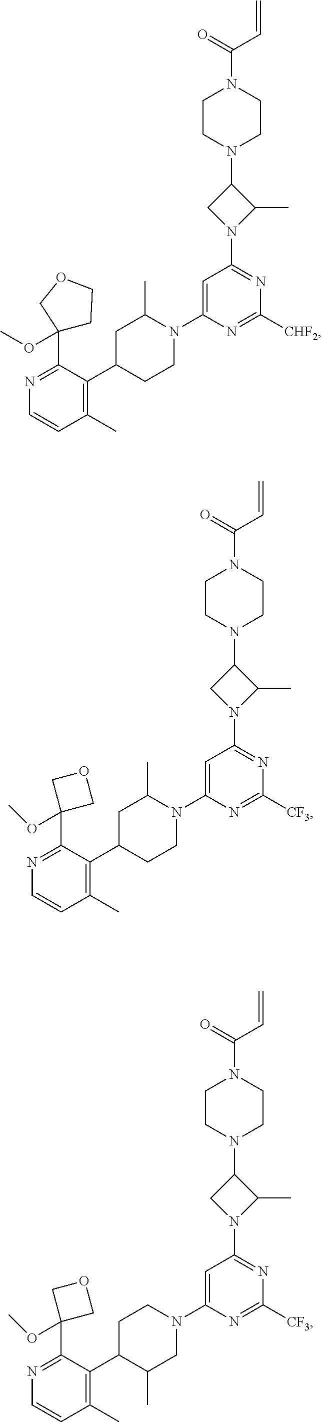 [SMILES: C=CC(=O)N1CCN(C2CN(c3cc(N4CCC(c5c(C)ccnc5C5(OC)CCOC5)CC4C)nc(C(F)F)n3)C2C)CC1 ; C=CC(=O)N1CCN(C2CN(c3cc(N4CCC(c5c(C)ccnc5C5(OC)COC5)CC4C)nc(C(F)(F)F)n3)C2C)CC1 ; C=CC(=O)N1CCN(C2CN(c3cc(N4CCC(c5c(C)ccnc5C5(OC)COC5)C(C)C4)nc(C(F)(F)F)n3)C2C)CC1]

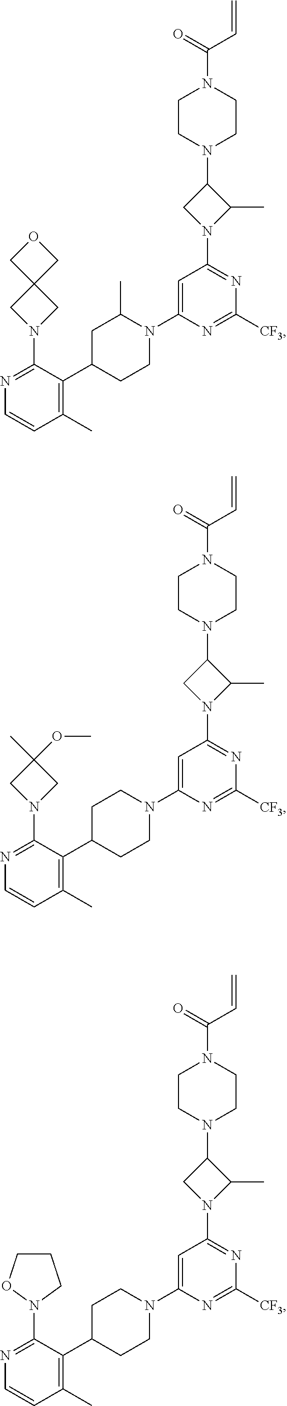 [SMILES: C=CC(=O)N1CCN(C2CN(c3cc(N4CCC(c5c(C)ccnc5N5CC6(COC6)C5)CC4C)nc(C(F)(F)F)n3)C2C)CC1 ; C=CC(=O)N1CCN(C2CN(c3cc(N4CCC(c5c(C)ccnc5N5CC(C)(OC)C5)CC4)nc(C(F)(F)F)n3)C2C)CC1 ; C=CC(=O)N1CCN(C2CN(c3cc(N4CCC(c5c(C)ccnc5N5CCCO5)CC4)nc(C(F)(F)F)n3)C2C)CC1]

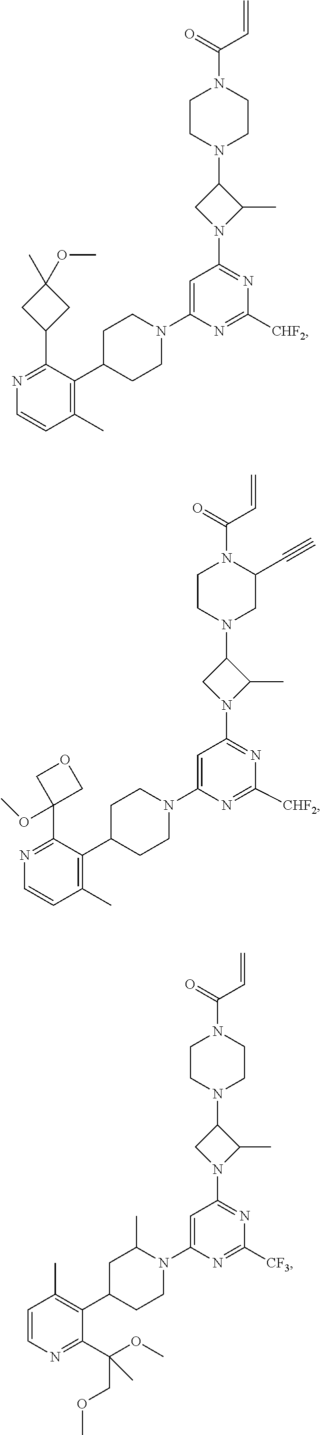 [SMILES: C=CC(=O)N1CCN(C2CN(c3cc(N4CCC(c5c(C)ccnc5C5CC(C)(OC)C5)CC4)nc(C(F)F)n3)C2C)CC1 ; C#CC1CN(C2CN(c3cc(N4CCC(c5c(C)ccnc5C5(OC)COC5)CC4)nc(C(F)F)n3)C2C)CCN1C(=O)C=C ; C=CC(=O)N1CCN(C2CN(c3cc(N4CCC(c5c(C)ccnc5C(C)(COC)OC)CC4C)nc(C(F)(F)F)n3)C2C)CC1]

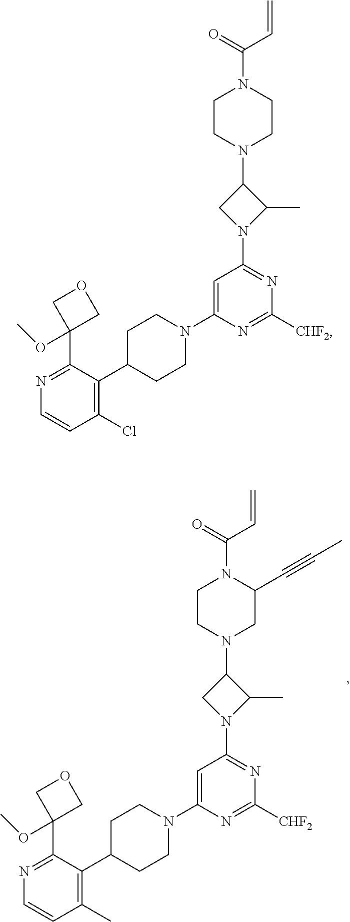 [SMILES: C=CC(=O)N1CCN(C2CN(c3cc(N4CCC(c5c(Cl)ccnc5C5(OC)COC5)CC4)nc(C(F)F)n3)C2C)CC1 ; C=CC(=O)N1CCN(C2CN(c3cc(N4CCC(c5c(C)ccnc5C5(OC)COC5)CC4)nc(C(F)F)n3)C2C)CC1C#CC]

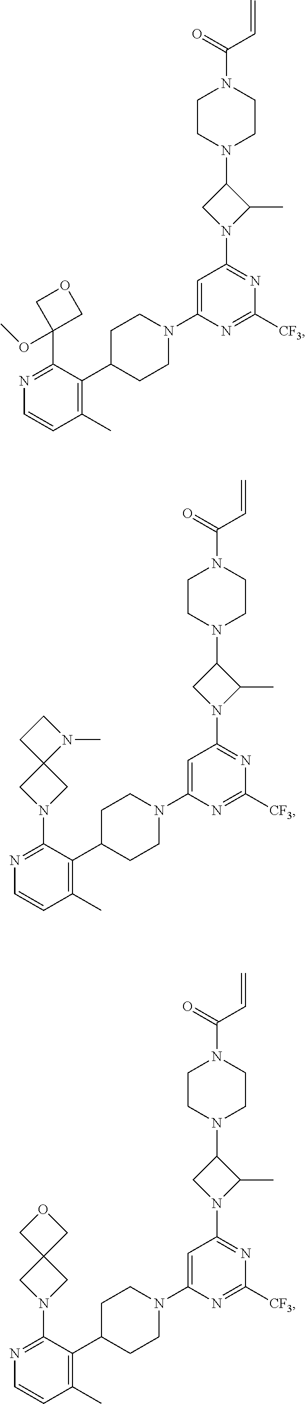 [SMILES: C=CC(=O)N1CCN(C2CN(c3cc(N4CCC(c5c(C)ccnc5C5(OC)COC5)CC4)nc(C(F)(F)F)n3)C2C)CC1 ; C=CC(=O)N1CCN(C2CN(c3cc(N4CCC(c5c(C)ccnc5N5CC6(CCN6C)C5)CC4)nc(C(F)(F)F)n3)C2C)CC1 ; C=CC(=O)N1CCN(C2CN(c3cc(N4CCC(c5c(C)ccnc5N5CC6(COC6)C5)CC4)nc(C(F)(F)F)n3)C2C)CC1]

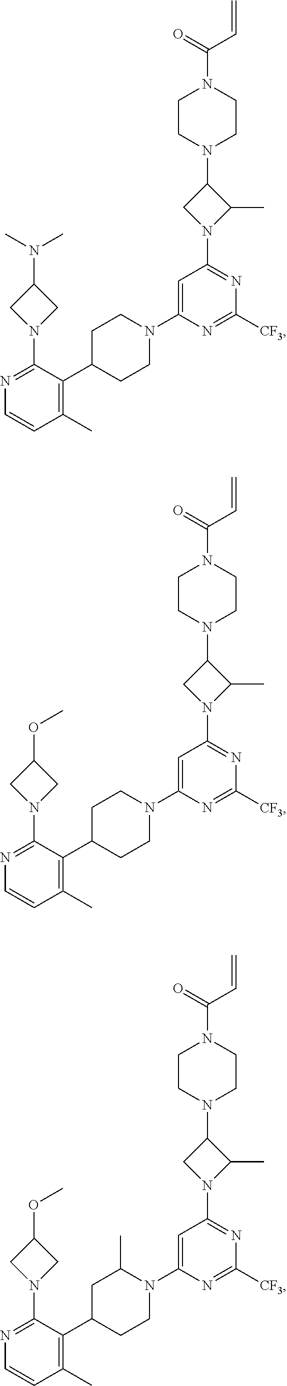 [SMILES: C=CC(=O)N1CCN(C2CN(c3cc(N4CCC(c5c(C)ccnc5N5CC(N(C)C)C5)CC4)nc(C(F)(F)F)n3)C2C)CC1 ; C=CC(=O)N1CCN(C2CN(c3cc(N4CCC(c5c(C)ccnc5N5CC(OC)C5)CC4)nc(C(F)(F)F)n3)C2C)CC1 ; C=CC(=O)N1CCN(C2CN(c3cc(N4CCC(c5c(C)ccnc5N5CC(OC)C5)CC4C)nc(C(F)(F)F)n3)C2C)CC1]

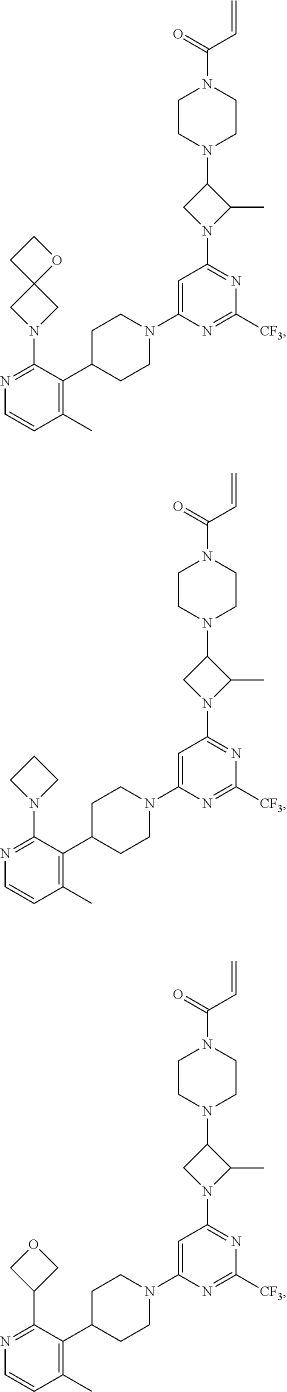 [SMILES: C=CC(=O)N1CCN(C2CN(c3cc(N4CCC(c5c(C)ccnc5N5CC6(CCO6)C5)CC4)nc(C(F)(F)F)n3)C2C)CC1 ; C=CC(=O)N1CCN(C2CN(c3cc(N4CCC(c5c(C)ccnc5N5CCC5)CC4)nc(C(F)(F)F)n3)C2C)CC1 ; C=CC(=O)N1CCN(C2CN(c3cc(N4CCC(c5c(C)ccnc5C5COC5)CC4)nc(C(F)(F)F)n3)C2C)CC1]

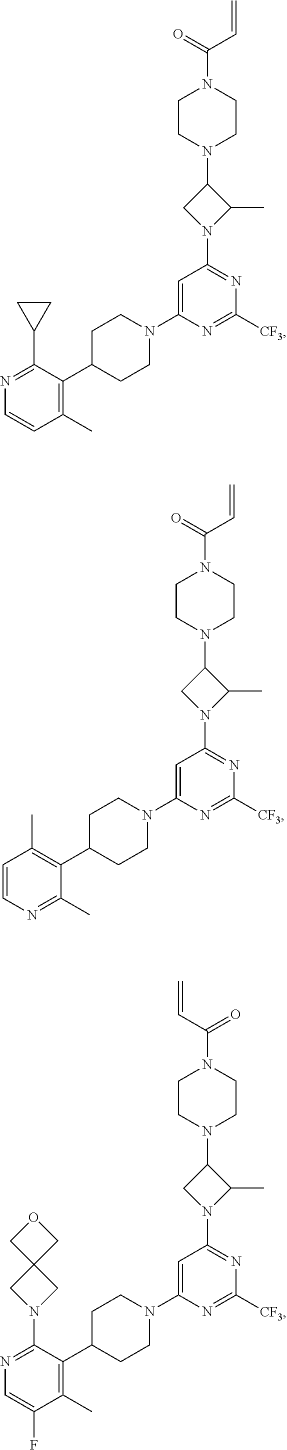 [SMILES: C=CC(=O)N1CCN(C2CN(c3cc(N4CCC(c5c(C)ccnc5C5CC5)CC4)nc(C(F)(F)F)n3)C2C)CC1 ; C=CC(=O)N1CCN(C2CN(c3cc(N4CCC(c5c(C)ccnc5C)CC4)nc(C(F)(F)F)n3)C2C)CC1 ; C=CC(=O)N1CCN(C2CN(c3cc(N4CCC(c5c(N6CC7(COC7)C6)ncc(F)c5C)CC4)nc(C(F)(F)F)n3)C2C)CC1]

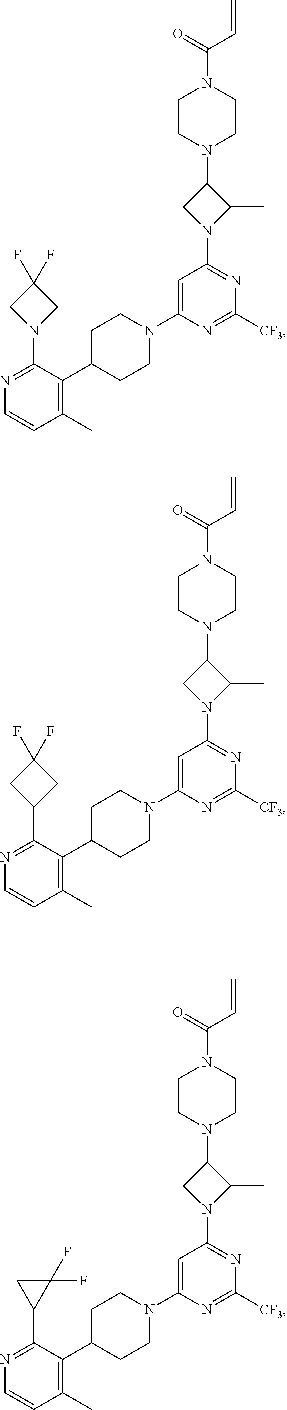 [SMILES: C=CC(=O)N1CCN(C2CN(c3cc(N4CCC(c5c(C)ccnc5N5CC(F)(F)C5)CC4)nc(C(F)(F)F)n3)C2C)CC1 ; C=CC(=O)N1CCN(C2CN(c3cc(N4CCC(c5c(C)ccnc5C5CC(F)(F)C5)CC4)nc(C(F)(F)F)n3)C2C)CC1 ; C=CC(=O)N1CCN(C2CN(c3cc(N4CCC(c5c(C)ccnc5C5CC5(F)F)CC4)nc(C(F)(F)F)n3)C2C)CC1]

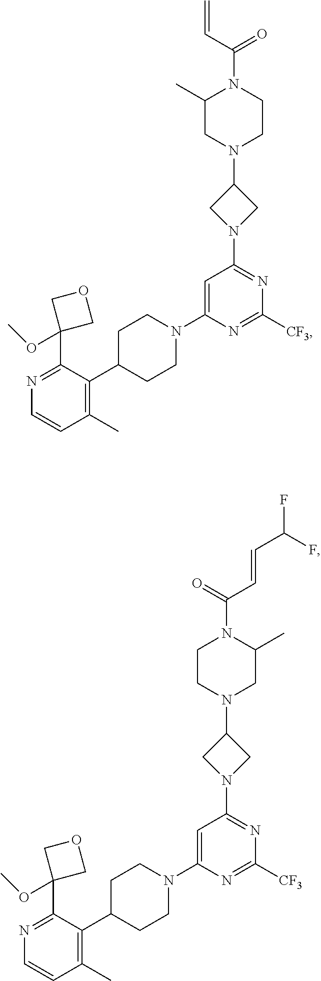 [SMILES: C=CC(=O)N1CCN(C2CN(c3cc(N4CCC(c5c(C)ccnc5C5(OC)COC5)CC4)nc(C(F)(F)F)n3)C2)CC1C ; COC1(c2nccc(C)c2C2CCN(c3cc(N4CC(N5CCN(C(=O)/C=C/C(F)F)C(C)C5)C4)nc(C(F)(F)F)n3)CC2)COC1]

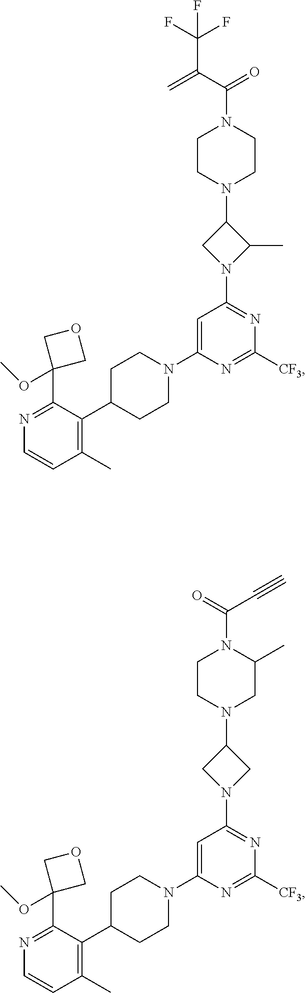 [SMILES: C=C(C(=O)N1CCN(C2CN(c3cc(N4CCC(c5c(C)ccnc5C5(OC)COC5)CC4)nc(C(F)(F)F)n3)C2C)CC1)C(F)(F)F ; C#CC(=O)N1CCN(C2CN(c3cc(N4CCC(c5c(C)ccnc5C5(OC)COC5)CC4)nc(C(F)(F)F)n3)C2)CC1C]

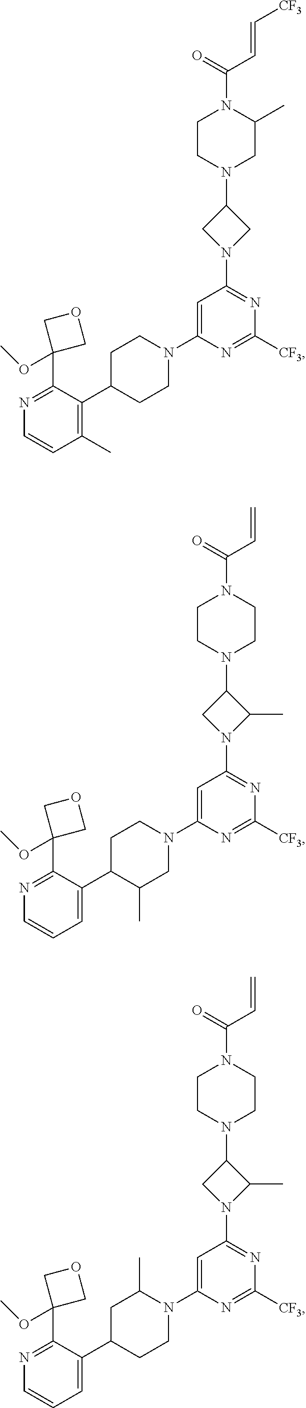 [SMILES: COC1(c2nccc(C)c2C2CCN(c3cc(N4CC(N5CCN(C(=O)/C=C/C(F)(F)F)C(C)C5)C4)nc(C(F)(F)F)n3)CC2)COC1 ; C=CC(=O)N1CCN(C2CN(c3cc(N4CCC(c5cccnc5C5(OC)COC5)C(C)C4)nc(C(F)(F)F)n3)C2C)CC1 ; C=CC(=O)N1CCN(C2CN(c3cc(N4CCC(c5cccnc5C5(OC)COC5)CC4C)nc(C(F)(F)F)n3)C2C)CC1]

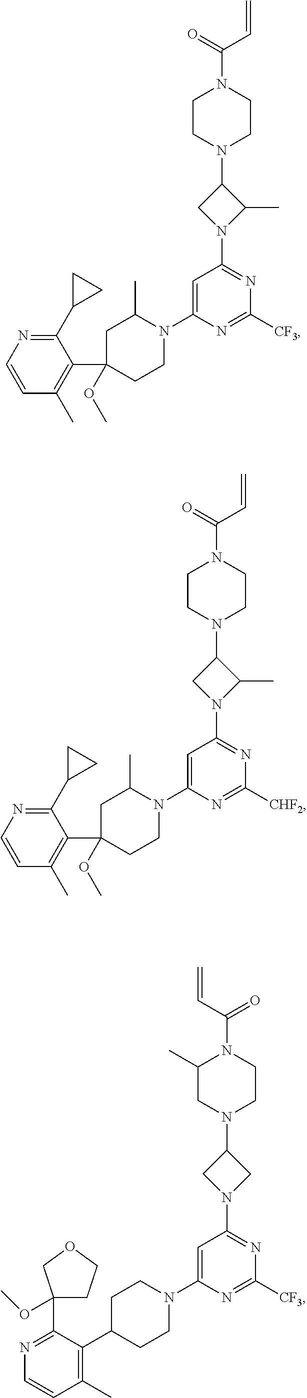 [SMILES: C=CC(=O)N1CCN(C2CN(c3cc(N4CCC(OC)(c5c(C)ccnc5C5CC5)CC4C)nc(C(F)(F)F)n3)C2C)CC1 ; C=CC(=O)N1CCN(C2CN(c3cc(N4CCC(OC)(c5c(C)ccnc5C5CC5)CC4C)nc(C(F)F)n3)C2C)CC1 ; C=CC(=O)N1CCN(C2CN(c3cc(N4CCC(c5c(C)ccnc5C5(OC)CCOC5)CC4)nc(C(F)(F)F)n3)C2)CC1C]

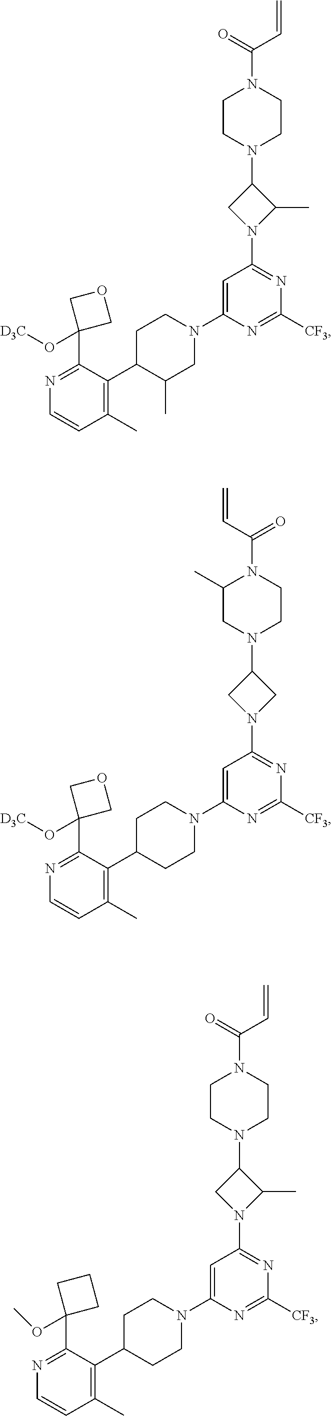 [SMILES: [2H]C([2H])([2H])OC1(c2nccc(C)c2C2CCN(c3cc(N4CC(N5CCN(C(=O)C=C)CC5)C4C)nc(C(F)(F)F)n3)CC2C)COC1 ; [2H]C([2H])([2H])OC1(c2nccc(C)c2C2CCN(c3cc(N4CC(N5CCN(C(=O)C=C)C(C)C5)C4)nc(C(F)(F)F)n3)CC2)COC1 ; C=CC(=O)N1CCN(C2CN(c3cc(N4CCC(c5c(C)ccnc5C5(OC)CCC5)CC4)nc(C(F)(F)F)n3)C2C)CC1]

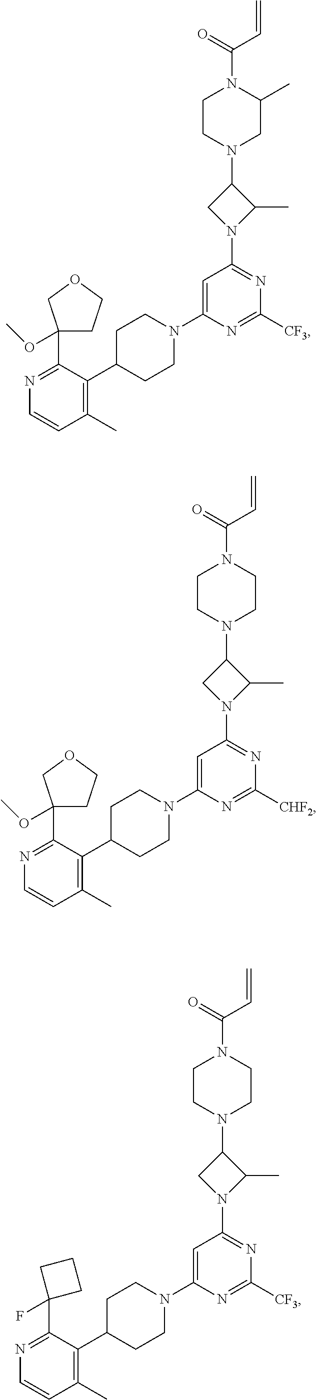 [SMILES: C=CC(=O)N1CCN(C2CN(c3cc(N4CCC(c5c(C)ccnc5C5(OC)CCOC5)CC4)nc(C(F)(F)F)n3)C2C)CC1C ; C=CC(=O)N1CCN(C2CN(c3cc(N4CCC(c5c(C)ccnc5C5(OC)CCOC5)CC4)nc(C(F)F)n3)C2C)CC1 ; C=CC(=O)N1CCN(C2CN(c3cc(N4CCC(c5c(C)ccnc5C5(F)CCC5)CC4)nc(C(F)(F)F)n3)C2C)CC1]

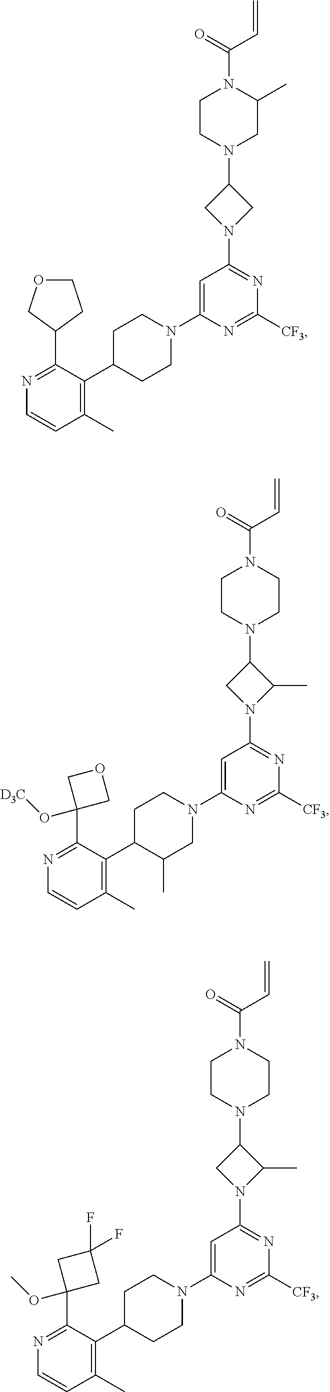 [SMILES: C=CC(=O)N1CCN(C2CN(c3cc(N4CCC(c5c(C)ccnc5C5CCOC5)CC4)nc(C(F)(F)F)n3)C2)CC1C ; [2H]C([2H])([2H])OC1(c2nccc(C)c2C2CCN(c3cc(N4CC(N5CCN(C(=O)C=C)CC5)C4C)nc(C(F)(F)F)n3)CC2C)COC1 ; C=CC(=O)N1CCN(C2CN(c3cc(N4CCC(c5c(C)ccnc5C5(OC)CC(F)(F)C5)CC4)nc(C(F)(F)F)n3)C2C)CC1]

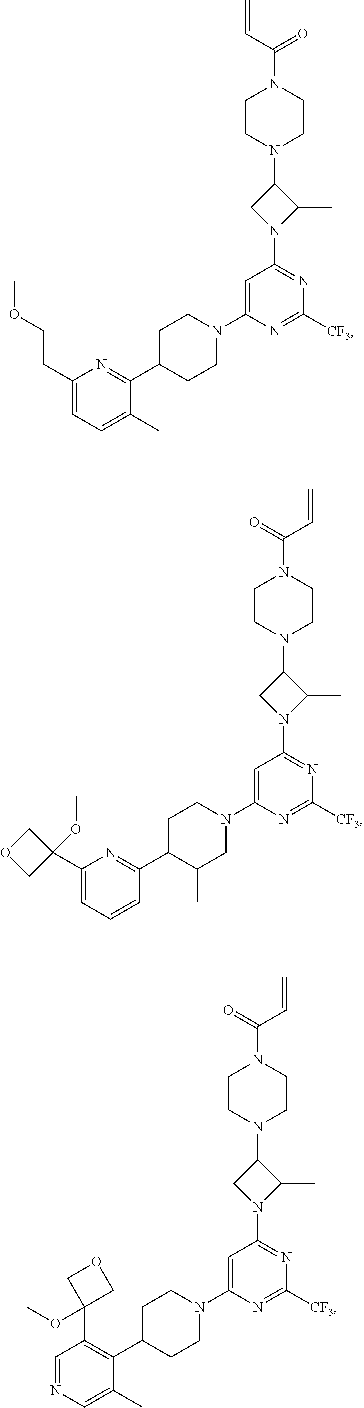 [SMILES: C=CC(=O)N1CCN(C2CN(c3cc(N4CCC(c5nc(CCOC)ccc5C)CC4)nc(C(F)(F)F)n3)C2C)CC1 ; C=CC(=O)N1CCN(C2CN(c3cc(N4CCC(c5cccc(C6(OC)COC6)n5)C(C)C4)nc(C(F)(F)F)n3)C2C)CC1 ; C=CC(=O)N1CCN(C2CN(c3cc(N4CCC(c5c(C)cncc5C5(OC)COC5)CC4)nc(C(F)(F)F)n3)C2C)CC1]

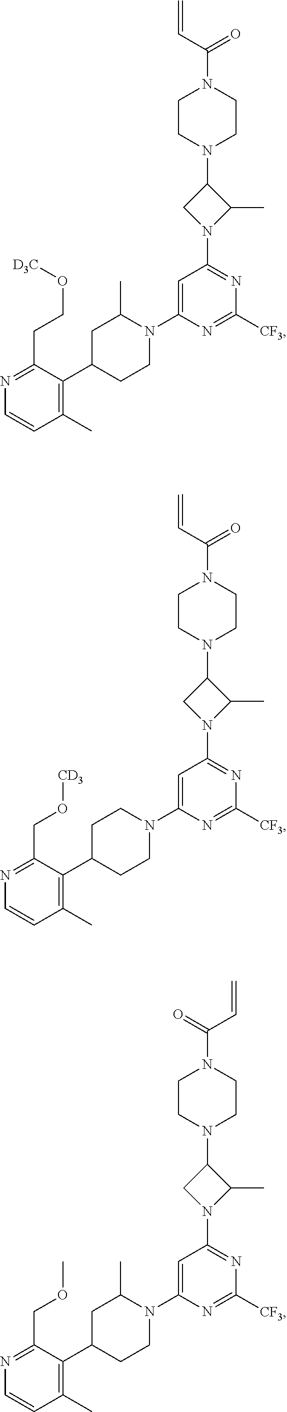 [SMILES: [2H]C([2H])([2H])OCCc1nccc(C)c1C1CCN(c2cc(N3CC(N4CCN(C(=O)C=C)CC4)C3C)nc(C(F)(F)F)n2)C(C)C1 ; [2H]C([2H])([2H])OCc1nccc(C)c1C1CCN(c2cc(N3CC(N4CCN(C(=O)C=C)CC4)C3C)nc(C(F)(F)F)n2)CC1 ; C=CC(=O)N1CCN(C2CN(c3cc(N4CCC(c5c(C)ccnc5COC)CC4C)nc(C(F)(F)F)n3)C2C)CC1]

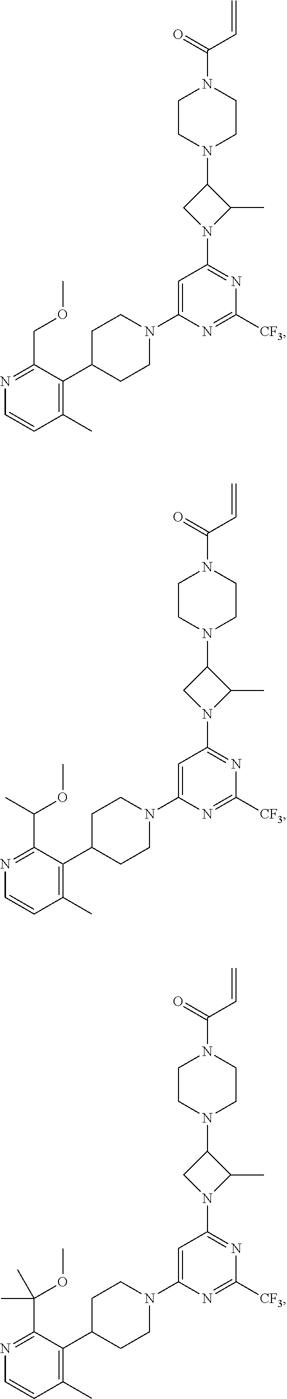 [SMILES: C=CC(=O)N1CCN(C2CN(c3cc(N4CCC(c5c(C)ccnc5COC)CC4)nc(C(F)(F)F)n3)C2C)CC1 ; C=CC(=O)N1CCN(C2CN(c3cc(N4CCC(c5c(C)ccnc5C(C)OC)CC4)nc(C(F)(F)F)n3)C2C)CC1 ; C=CC(=O)N1CCN(C2CN(c3cc(N4CCC(c5c(C)ccnc5C(C)(C)OC)CC4)nc(C(F)(F)F)n3)C2C)CC1]

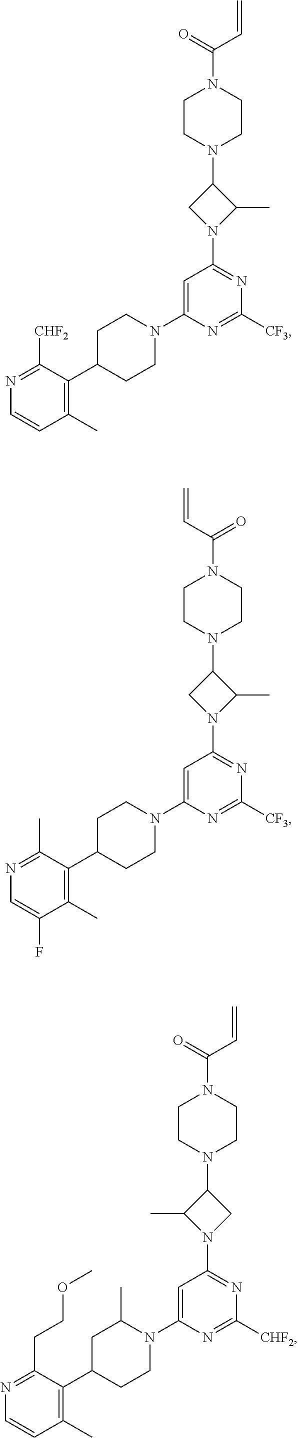 [SMILES: C=CC(=O)N1CCN(C2CN(c3cc(N4CCC(c5c(C)ccnc5C(F)F)CC4)nc(C(F)(F)F)n3)C2C)CC1 ; C=CC(=O)N1CCN(C2CN(c3cc(N4CCC(c5c(C)ncc(F)c5C)CC4)nc(C(F)(F)F)n3)C2C)CC1 ; C=CC(=O)N1CCN(C2CN(c3cc(N4CCC(c5c(C)ccnc5CCOC)CC4C)nc(C(F)F)n3)C2C)CC1]

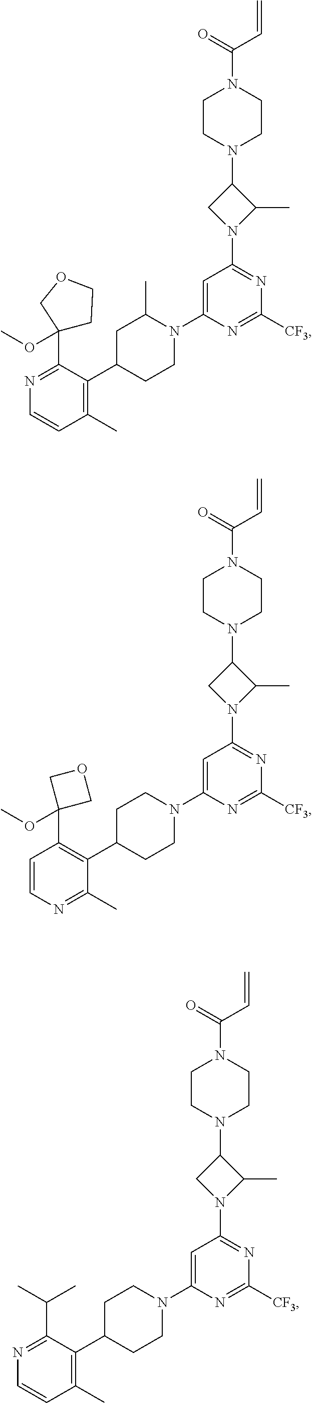 [SMILES: C=CC(=O)N1CCN(C2CN(c3cc(N4CCC(c5c(C)ccnc5C5(OC)CCOC5)CC4C)nc(C(F)(F)F)n3)C2C)CC1 ; C=CC(=O)N1CCN(C2CN(c3cc(N4CCC(c5c(C6(OC)COC6)ccnc5C)CC4)nc(C(F)(F)F)n3)C2C)CC1 ; C=CC(=O)N1CCN(C2CN(c3cc(N4CCC(c5c(C)ccnc5C(C)C)CC4)nc(C(F)(F)F)n3)C2C)CC1]

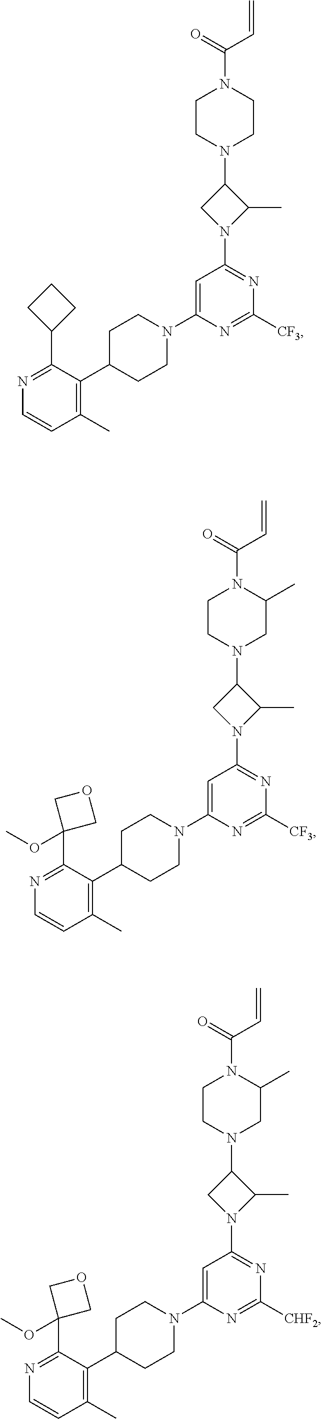 [SMILES: C=CC(=O)N1CCN(C2CN(c3cc(N4CCC(c5c(C)ccnc5C5CCC5)CC4)nc(C(F)(F)F)n3)C2C)CC1 ; C=CC(=O)N1CCN(C2CN(c3cc(N4CCC(c5c(C)ccnc5C5(OC)COC5)CC4)nc(C(F)(F)F)n3)C2C)CC1C ; C=CC(=O)N1CCN(C2CN(c3cc(N4CCC(c5c(C)ccnc5C5(OC)COC5)CC4)nc(C(F)F)n3)C2C)CC1C]

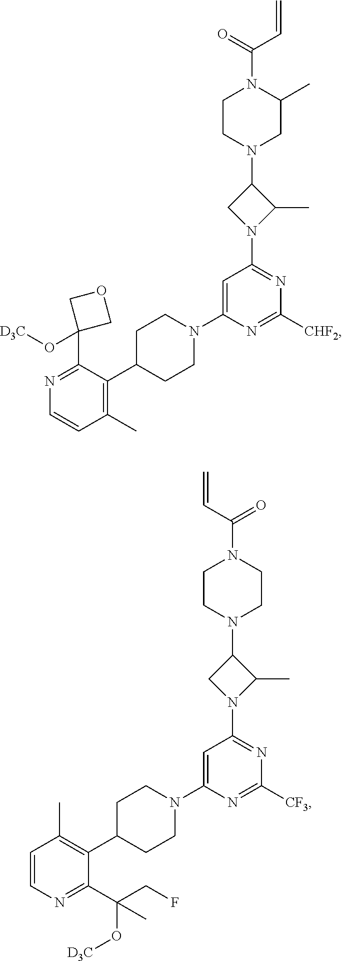 [SMILES: [2H]C([2H])([2H])OC1(c2nccc(C)c2C2CCN(c3cc(N4CC(N5CCN(C(=O)C=C)C(C)C5)C4C)nc(C(F)F)n3)CC2)COC1 ; [2H]C([2H])([2H])OC(C)(CF)c1nccc(C)c1C1CCN(c2cc(N3CC(N4CCN(C(=O)C=C)CC4)C3C)nc(C(F)(F)F)n2)CC1]

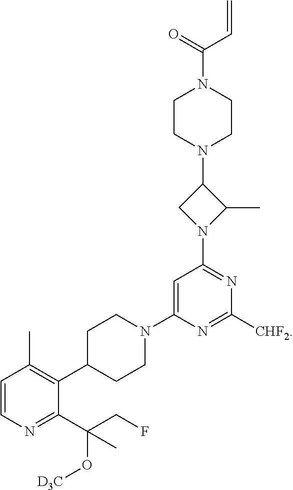 [SMILES: [2H]C([2H])([2H])OC(C)(CF)c1nccc(C)c1C1CCN(c2cc(N3CC(N4CCN(C(=O)C=C)CC4)C3C)nc(C(F)F)n2)CC1]

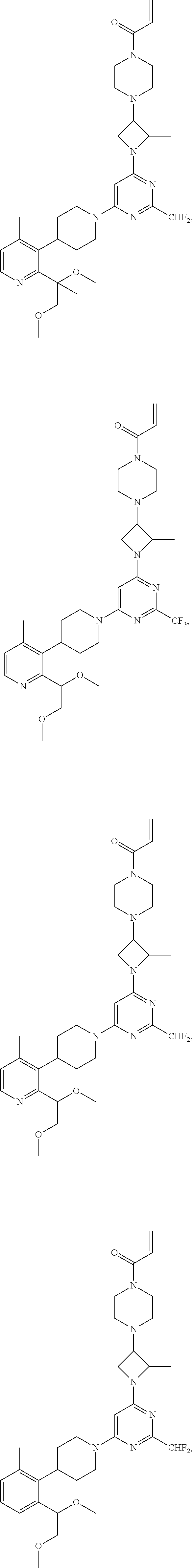 [SMILES: C=CC(=O)N1CCN(C2CN(c3cc(N4CCC(c5c(C)ccnc5C(C)(COC)OC)CC4)nc(C(F)F)n3)C2C)CC1 ; C=CC(=O)N1CCN(C2CN(c3cc(N4CCC(c5c(C)ccnc5C(COC)OC)CC4)nc(C(F)(F)F)n3)C2C)CC1 ; C=CC(=O)N1CCN(C2CN(c3cc(N4CCC(c5c(C)ccnc5C(COC)OC)CC4)nc(C(F)F)n3)C2C)CC1 ; C=CC(=O)N1CCN(C2CN(c3cc(N4CCC(c5c(C)cccc5C(COC)OC)CC4)nc(C(F)F)n3)C2C)CC1]

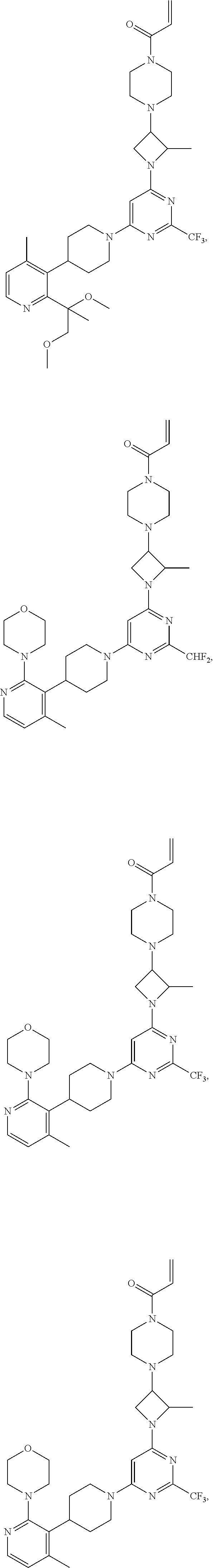 [SMILES: C=CC(=O)N1CCN(C2CN(c3cc(N4CCC(c5c(C)ccnc5C(C)(COC)OC)CC4)nc(C(F)(F)F)n3)C2C)CC1 ; C=CC(=O)N1CCN(C2CN(c3cc(N4CCC(c5c(C)ccnc5N5CCOCC5)CC4)nc(C(F)F)n3)C2C)CC1 ; C=CC(=O)N1CCN(C2CN(c3cc(N4CCC(c5c(C)ccnc5N5CCOCC5)CC4)nc(C(F)(F)F)n3)C2C)CC1 ; C=CC(=O)N1CCN(C2CN(c3cc(N4CCC(c5c(C)ccnc5N5CCOCC5)CC4)nc(C(F)(F)F)n3)C2C)CC1]

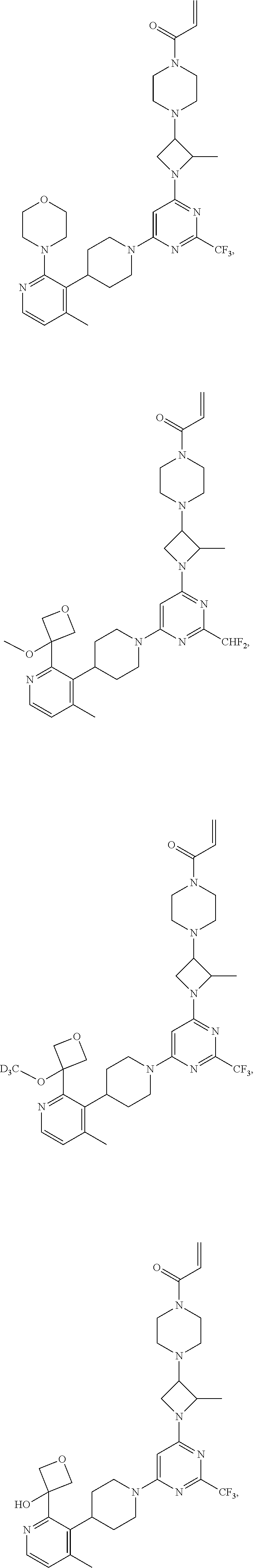 [SMILES: C=CC(=O)N1CCN(C2CN(c3cc(N4CCC(c5c(C)ccnc5N5CCOCC5)CC4)nc(C(F)(F)F)n3)C2C)CC1 ; C=CC(=O)N1CCN(C2CN(c3cc(N4CCC(c5c(C)ccnc5C5(OC)COC5)CC4)nc(C(F)F)n3)C2C)CC1 ; [2H]C([2H])([2H])OC1(c2nccc(C)c2C2CCN(c3cc(N4CC(N5CCN(C(=O)C=C)CC5)C4C)nc(C(F)(F)F)n3)CC2)COC1 ; C=CC(=O)N1CCN(C2CN(c3cc(N4CCC(c5c(C)ccnc5C5(O)COC5)CC4)nc(C(F)(F)F)n3)C2C)CC1]

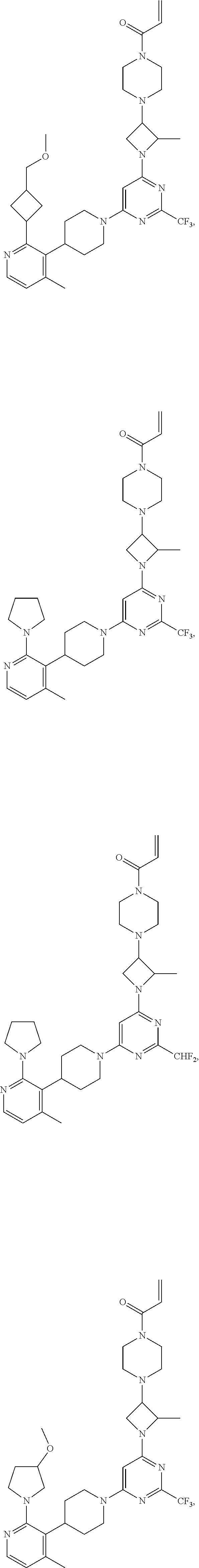 [SMILES: C=CC(=O)N1CCN(C2CN(c3cc(N4CCC(c5c(C)ccnc5C5CC(COC)C5)CC4)nc(C(F)(F)F)n3)C2C)CC1 ; C=CC(=O)N1CCN(C2CN(c3cc(N4CCC(c5c(C)ccnc5N5CCCC5)CC4)nc(C(F)(F)F)n3)C2C)CC1 ; C=CC(=O)N1CCN(C2CN(c3cc(N4CCC(c5c(C)ccnc5N5CCCC5)CC4)nc(C(F)F)n3)C2C)CC1 ; C=CC(=O)N1CCN(C2CN(c3cc(N4CCC(c5c(C)ccnc5N5CCC(OC)C5)CC4)nc(C(F)(F)F)n3)C2C)CC1]

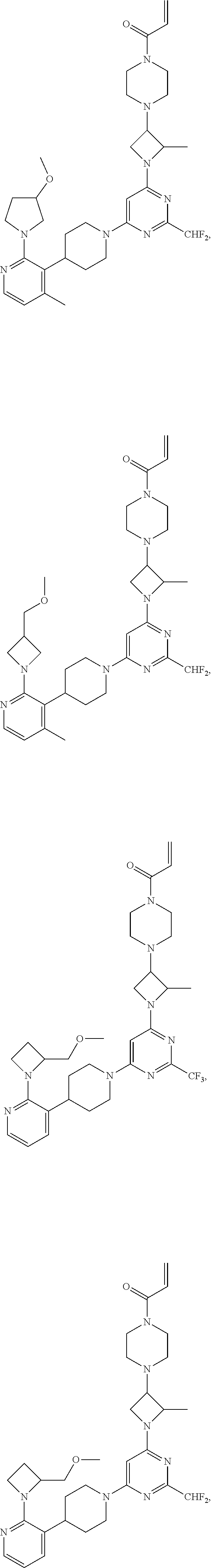 [SMILES: C=CC(=O)N1CCN(C2CN(c3cc(N4CCC(c5c(C)ccnc5N5CCC(OC)C5)CC4)nc(C(F)F)n3)C2C)CC1 ; C=CC(=O)N1CCN(C2CN(c3cc(N4CCC(c5c(C)ccnc5N5CC(COC)C5)CC4)nc(C(F)F)n3)C2C)CC1 ; C=CC(=O)N1CCN(C2CN(c3cc(N4CCC(c5cccnc5N5CCC5COC)CC4)nc(C(F)(F)F)n3)C2C)CC1 ; C=CC(=O)N1CCN(C2CN(c3cc(N4CCC(c5cccnc5N5CCC5COC)CC4)nc(C(F)F)n3)C2C)CC1]

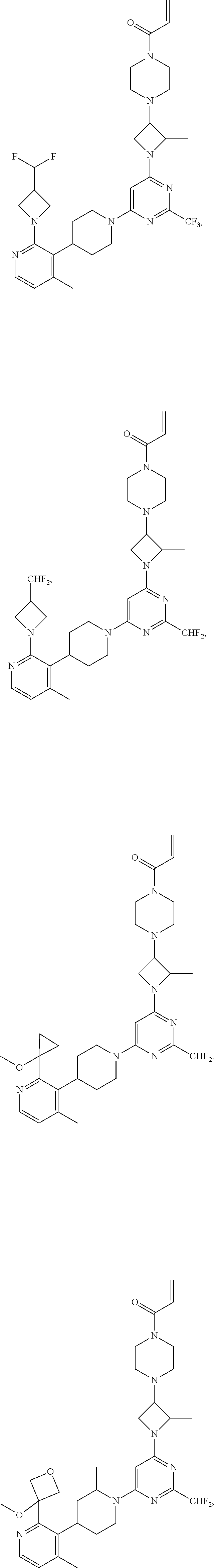 [SMILES: C=CC(=O)N1CCN(C2CN(c3cc(N4CCC(c5c(C)ccnc5N5CC(C(F)F)C5)CC4)nc(C(F)(F)F)n3)C2C)CC1 ; C=CC(=O)N1CCN(C2CN(c3cc(N4CCC(c5c(C)ccnc5N5CC(C(F)F)C5)CC4)nc(C(F)F)n3)C2C)CC1 ; C=CC(=O)N1CCN(C2CN(c3cc(N4CCC(c5c(C)ccnc5C5(OC)CC5)CC4)nc(C(F)F)n3)C2C)CC1 ; C=CC(=O)N1CCN(C2CN(c3cc(N4CCC(c5c(C)ccnc5C5(OC)COC5)CC4C)nc(C(F)F)n3)C2C)CC1]

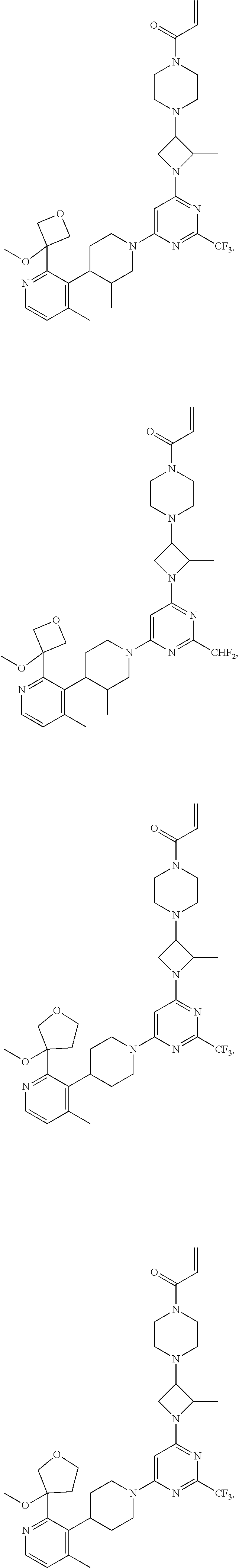 [SMILES: C=CC(=O)N1CCN(C2CN(c3cc(N4CCC(c5c(C)ccnc5C5(OC)COC5)C(C)C4)nc(C(F)(F)F)n3)C2C)CC1 ; C=CC(=O)N1CCN(C2CN(c3cc(N4CCC(c5c(C)ccnc5C5(OC)COC5)C(C)C4)nc(C(F)F)n3)C2C)CC1 ; C=CC(=O)N1CCN(C2CN(c3cc(N4CCC(c5c(C)ccnc5C5(OC)CCOC5)CC4)nc(C(F)(F)F)n3)C2C)CC1 ; C=CC(=O)N1CCN(C2CN(c3cc(N4CCC(c5c(C)ccnc5C5(OC)CCOC5)CC4)nc(C(F)(F)F)n3)C2C)CC1]

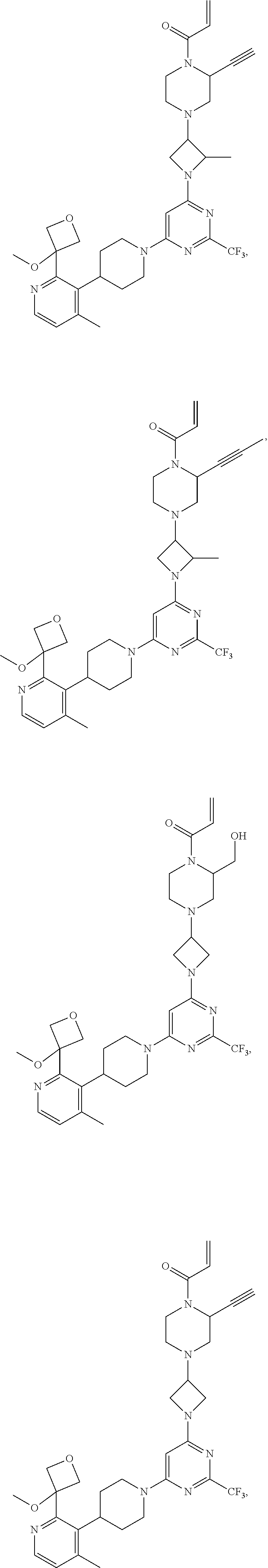

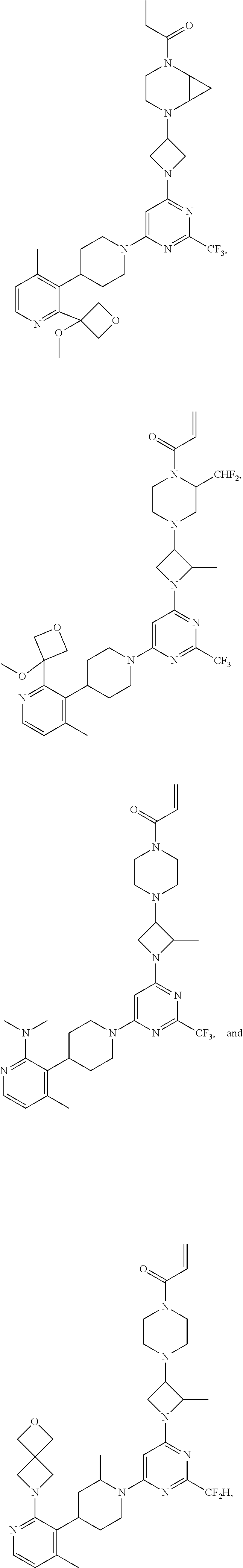 [SMILES: CCC(=O)N1CCN(C2CN(c3cc(N4CCC(c5c(C)ccnc5C5(OC)COC5)CC4)nc(C(F)(F)F)n3)C2)C2CC21 ; C=CC(=O)N1CCN(C2CN(c3cc(N4CCC(c5c(C)ccnc5C5(OC)COC5)CC4)nc(C(F)(F)F)n3)C2C)CC1C(F)F ; C=CC(=O)N1CCN(C2CN(c3cc(N4CCC(c5c(C)ccnc5N(C)C)CC4)nc(C(F)(F)F)n3)C2C)CC1 ; C=CC(=O)N1CCN(C2CN(c3cc(N4CCC(c5c(C)ccnc5N5CC6(COC6)C5)CC4C)nc(C(F)F)n3)C2C)CC1]

and pharmaceutically acceptable salts thereof.

In some cases, the disclosure provides compounds of Formula (I) wherein A is N; X is

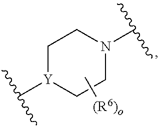

and Z is optionally substituted pyrazinyl. Examples of such compounds include but are not limited to:

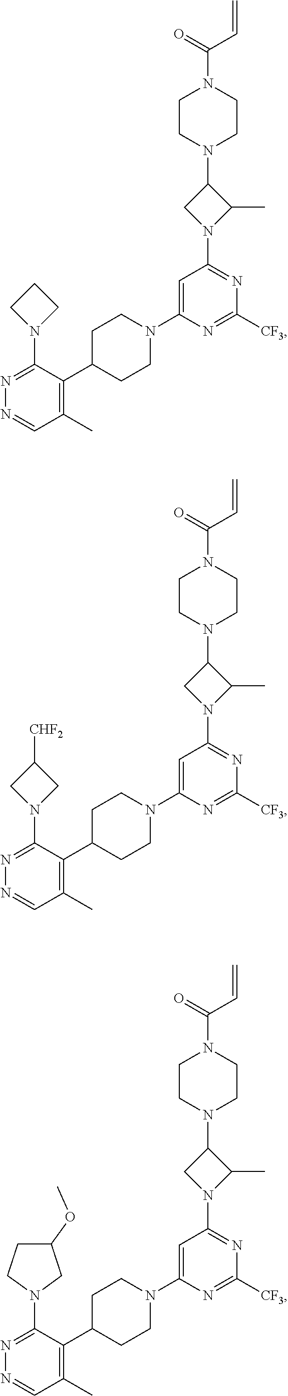 [SMILES: C=CC(=O)N1CCN(C2CN(c3cc(N4CCC(c5c(C)cnnc5N5CCC5)CC4)nc(C(F)(F)F)n3)C2C)CC1 ; C=CC(=O)N1CCN(C2CN(c3cc(N4CCC(c5c(C)cnnc5N5CC(C(F)F)C5)CC4)nc(C(F)(F)F)n3)C2C)CC1 ; C=CC(=O)N1CCN(C2CN(c3cc(N4CCC(c5c(C)cnnc5N5CCC(OC)C5)CC4)nc(C(F)(F)F)n3)C2C)CC1]

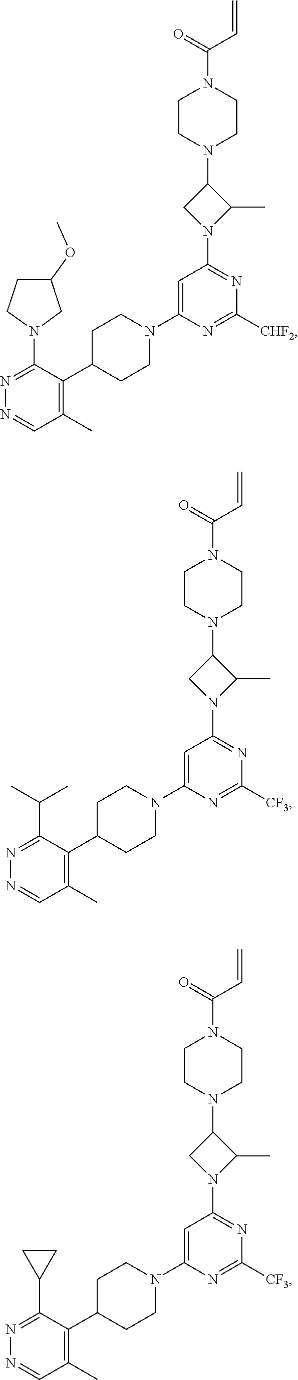 [SMILES: C=CC(=O)N1CCN(C2CN(c3cc(N4CCC(c5c(C)cnnc5N5CCC(OC)C5)CC4)nc(C(F)F)n3)C2C)CC1 ; C=CC(=O)N1CCN(C2CN(c3cc(N4CCC(c5c(C)cnnc5C(C)C)CC4)nc(C(F)(F)F)n3)C2C)CC1 ; C=CC(=O)N1CCN(C2CN(c3cc(N4CCC(c5c(C)cnnc5C5CC5)CC4)nc(C(F)(F)F)n3)C2C)CC1]

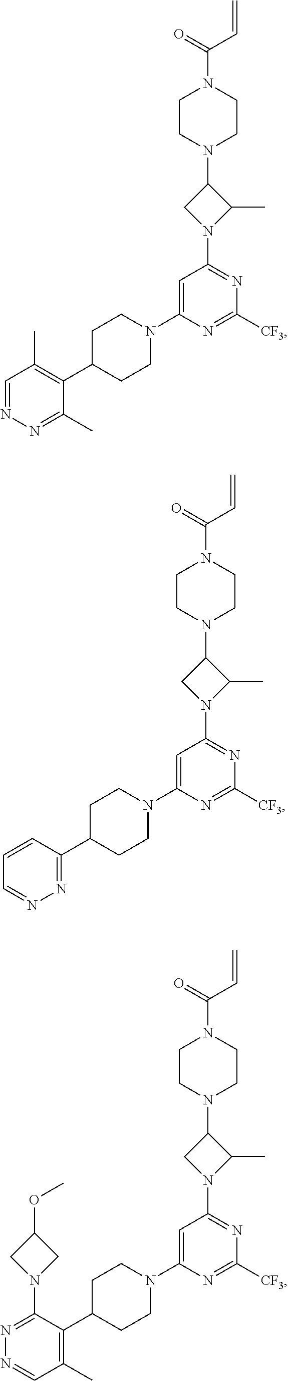 [SMILES: C=CC(=O)N1CCN(C2CN(c3cc(N4CCC(c5c(C)cnnc5C)CC4)nc(C(F)(F)F)n3)C2C)CC1 ; C=CC(=O)N1CCN(C2CN(c3cc(N4CCC(c5cccnn5)CC4)nc(C(F)(F)F)n3)C2C)CC1 ; C=CC(=O)N1CCN(C2CN(c3cc(N4CCC(c5c(C)cnnc5N5CC(OC)C5)CC4)nc(C(F)(F)F)n3)C2C)CC1]

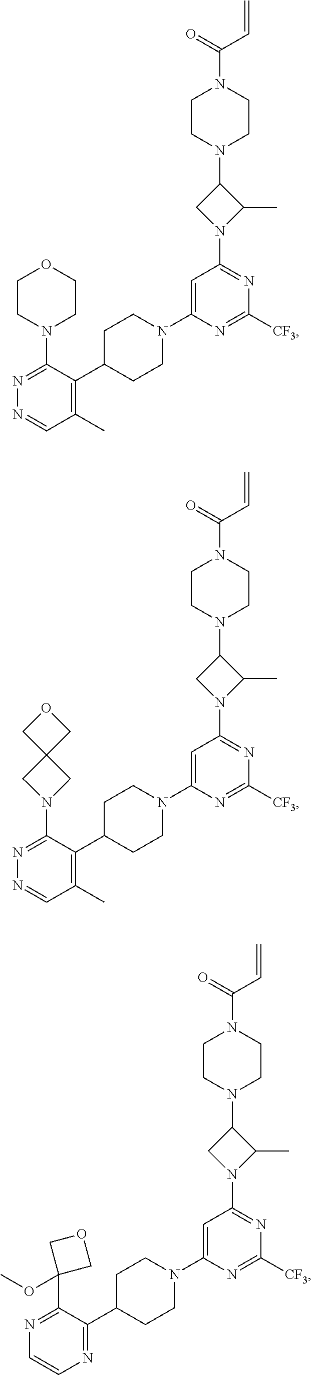 [SMILES: C=CC(=O)N1CCN(C2CN(c3cc(N4CCC(c5c(C)cnnc5N5CCOCC5)CC4)nc(C(F)(F)F)n3)C2C)CC1 ; C=CC(=O)N1CCN(C2CN(c3cc(N4CCC(c5c(C)cnnc5N5CC6(COC6)C5)CC4)nc(C(F)(F)F)n3)C2C)CC1 ; C=CC(=O)N1CCN(C2CN(c3cc(N4CCC(c5nccnc5C5(OC)COC5)CC4)nc(C(F)(F)F)n3)C2C)CC1]

and pharmaceutically acceptable salts thereof.

In some cases, the disclosure provides compounds of Formula (I) wherein A is N; X is

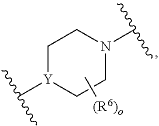

and Z is an optionally substituted bicyclic ring comprising a heteroaryl ring having 6 total ring atoms and 1-3 heteroatoms selected from N, O, and S fused to a heterocycloalkyl ring having 5 total ring atoms and 1 or 2 heteroatoms selected from N, O, and S. Examples of such compounds include but are not limited to:

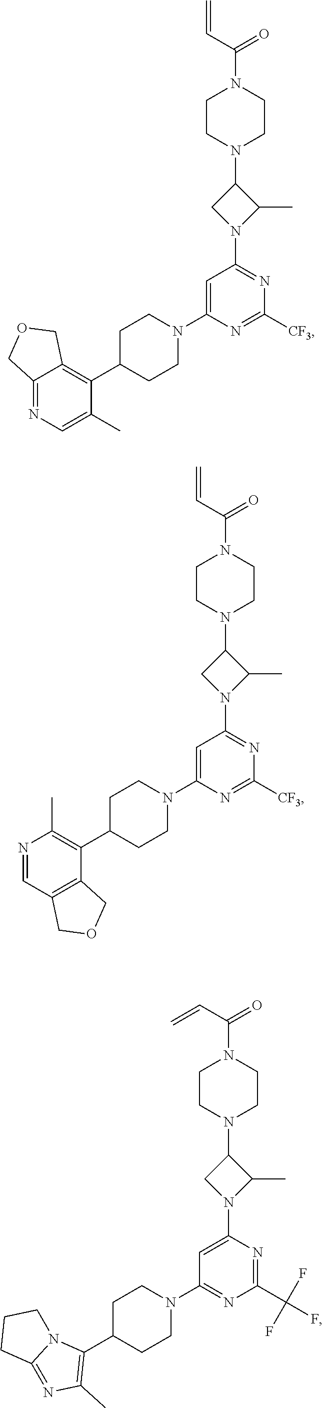 [SMILES: C=CC(=O)N1CCN(C2CN(c3cc(N4CCC(c5c(C)cnc6c5COC6)CC4)nc(C(F)(F)F)n3)C2C)CC1 ; C=CC(=O)N1CCN(C2CN(c3cc(N4CCC(c5c(C)ncc6c5COC6)CC4)nc(C(F)(F)F)n3)C2C)CC1 ; C=CC(=O)N1CCN(C2CN(c3cc(N4CCC(c5c(C)nc6n5CCC6)CC4)nc(C(F)(F)F)n3)C2C)CC1]

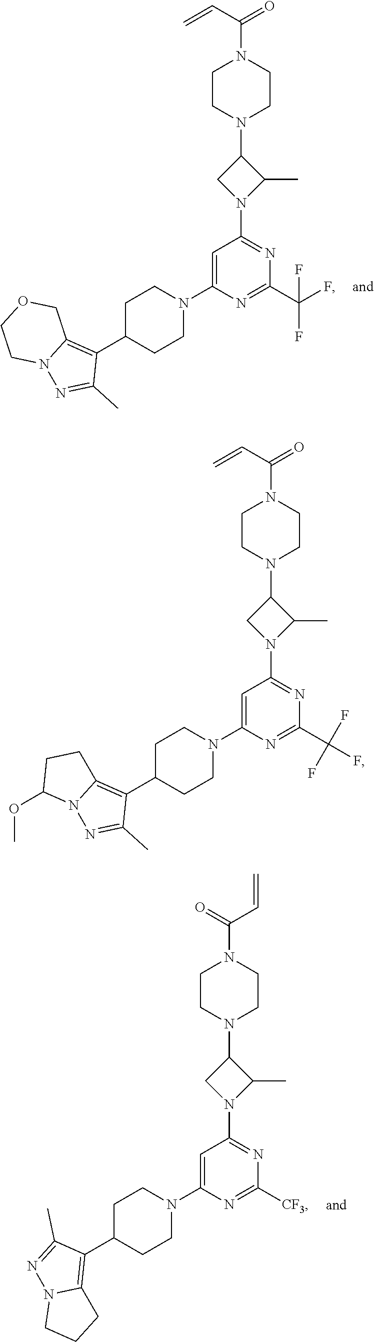 [SMILES: C=CC(=O)N1CCN(C2CN(c3cc(N4CCC(c5c(C)nn6c5COCC6)CC4)nc(C(F)(F)F)n3)C2C)CC1 ; "and" ; C=CC(=O)N1CCN(C2CN(c3cc(N4CCC(c5c(C)nn6c5CCC6OC)CC4)nc(C(F)(F)F)n3)C2C)CC1 ; C=CC(=O)N1CCN(C2CN(c3cc(N4CCC(c5c(C)nn6c5CCC6)CC4)nc(C(F)(F)F)n3)C2C)CC1 ; "and"]

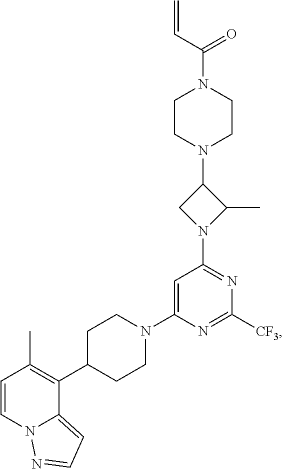 [SMILES: C=CC(=O)N1CCN(C2CN(c3cc(N4CCC(c5c(C)ccn6nccc56)CC4)nc(C(F)(F)F)n3)C2C)CC1]

and pharmaceutically acceptable salts thereof.

In some cases, the disclosure provides a compound listed in Table E, below if the stereochemistry of a structure or a portion of a structure in Table E is not explicitly shown (e.g., such as with dashed or bold lines), then the structure or portion of structure is either achiral or interpreted as being any of the possible stereoisomers of the structure or portion of the structure. In cases in which the stereochemistry of the structure or portion of the structure in Table E is explicitly shown, a single stereoisomer of the structure or portion of a structure is represented.

TABLE E

| Ex | Chemical Structure | Name | Ex. | Chemical Structure | Name |
|---|---|---|---|---|---|
| 2-001 | 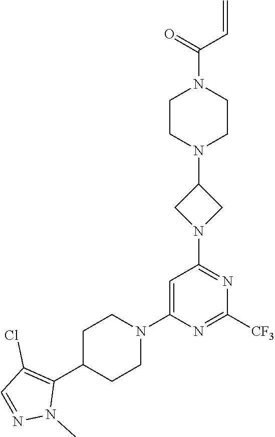 [SMILES: C=CC(=O)N1CCN(C2CN(c3cc(N4CCC(c5c(Cl)cnn5C)CC4)nc(C(F)(F)F)n3)C2)CC1] | 1-(4-(1-(6-(4-(4- chloro-1-methyl- 1H-pyrazol-5- yl)piperidin-1-yl)- 2-(trifluoromethyl) pyrimidin-4- yl)azetidin-3- yl)piperazin-1- yl)prop-2-en-1-one | 2-002 | 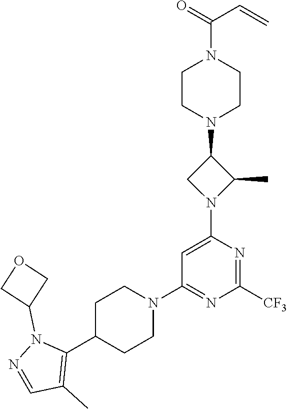 [SMILES: C=CC(=O)N1CCN([C@@H]2CN(c3cc(N4CCC(c5c(C)cnn5C5COC5)CC4)nc(C(F)(F)F)n3)[C@@H]2C)CC1] | 1-(4-((2R,3R)-2- methyl-1-(6-(4-(4- methyl-1-(oxetan- 3-yl)-1H-pyrazol- 5-yl)piperidin-1- yl)-2- (trifluoromethyl) pyrimidin-4- yl)azetidin-3- yl)piperazin-1- yl)prop-2-en-1-one |
| 2-003 | 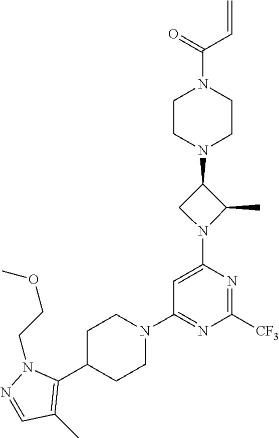 [SMILES: C=CC(=O)N1CCN([C@@H]2CN(c3cc(N4CCC(c5c(C)cnn5CCOC)CC4)nc(C(F)(F)F)n3)[C@@H]2C)CC1] | 1-(4-((2R,3R)-1-(6- (4-(1-(2- methoxyethyl)-4- methyl-1H- pyrazol-5- yl)piperidin-1-yl)- 2-(trifluoromethyl) pyrimidin-4-yl)-2- methylazetidin-3- yl)piperazin-1- yl)prop-2-en-1-one | 2-007 | 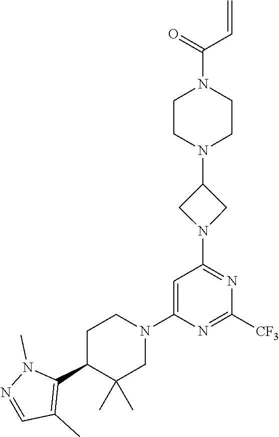 [SMILES: C=CC(=O)N1CCN(C2CN(c3cc(N4CC[C@H](c5c(C)cnn5C)C(C)(C)C4)nc(C(F)(F)F)n3)C2)CC1] | (S)-1-(4-(1-(6-(4- (1,4-dimethyl-1H- pyrazol-5-yl)-3,3- dimethylpiperidin- 1-yl)-2- (trifluoromethyl) pyrimidin-4- yl)azetidin-3- yl)piperazin-1- yl)prop-2-en-1-one |
| 2-004 | 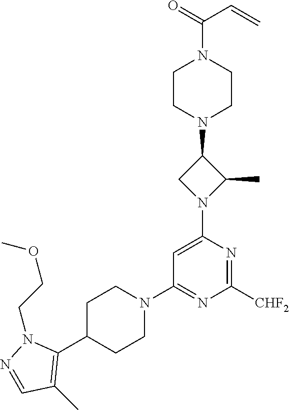 [SMILES: C=CC(=O)N1CCN([C@@H]2CN(c3cc(N4CCC(c5c(C)cnn5CCOC)CC4)nc(C(F)F)n3)[C@@H]2C)CC1] | 1-(4-((2R,3R)-1-(2- (difluoromethyl)-6- (4-(1-(2- methoxyethyl)-4. methyl-1H- pyrazol-5- yl)piperidin-1- yl)pyrimidin-4-yl)- 2-methylazetidin- 3-yl)piperazin-1- yl)prop-2-en-1-one | 2-008 | 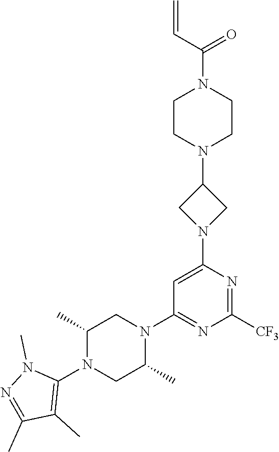 [SMILES: C=CC(=O)N1CCN(C2CN(c3cc(N4C[C@@H](C)N(c5c(C)c(C)nn5C)C[C@H]4C)nc(C(F)(F)F)n3)C2)CC1] | 1-(4-11-(6- ((2R,SR)-2,5- dimethyl-4-(1,3,4- trimethyl-1H- pyrazol-5- yl)piperazin-1-yl)- 2-(trifluoromethyl) pyrimidin-4- yl)azetidin-3- yl)piperazin-1- yl)prop-2-en-1-one |
| 2-005 | 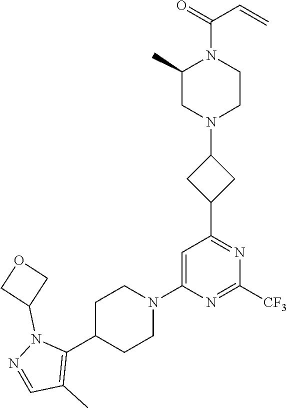 [SMILES: C=CC(=O)N1CCN(C2CC(c3cc(N4CCC(c5c(C)cnn5C5COC5)CC4)nc(C(F)(F)F)n3)C2)C[C@H]1C] | (R)-1-(2-methyl-4- (1-(6-(4-(4-methyl- 1-(oxetan-3-yl)- 1H-pyrazol-5- yl)piperidin-1-yl)- 2-(trifluoromethyl) pyrimidin-4- yljazetidin-3- yl)piperazin-1- yl)prop-2-en-1-one | 2-009 | 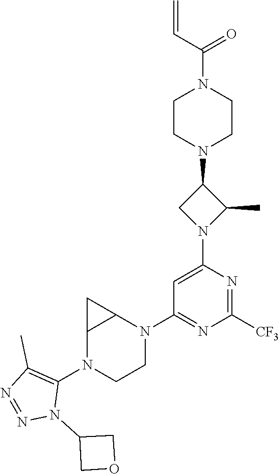 [SMILES: C=CC(=O)N1CCN([C@@H]2CN(c3cc(N4CCN(c5c(C)nnn5C5COC5)C5CC54)nc(C(F)(F)F)n3)[C@@H]2C)CC1] | 1-(4-((2R.3R)-2- methyl-1-(6-(5-(4- methyl-1-(oxetan- 3-yl)-1H-pyrazol- 5-y])-2,5- diazabicyclo[4.1.0] heptan-2-yl)-2- (trifluoromethyl) pyrimidin-4- yl)azetidin-3. yl)piperazin-1- yl)prop-2-en-1-one |
| 2-006 | 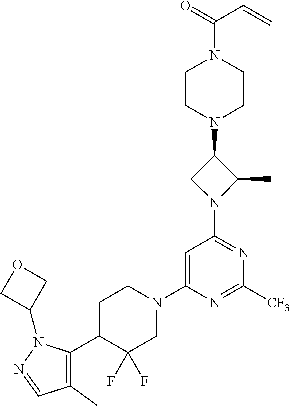 [SMILES: C=CC(=O)N1CCN([C@@H]2CN(c3cc(N4CCC(c5c(C)cnn5C5COC5)C(F)(F)C4)nc(C(F)(F)F)n3)[C@@H]2C)CC1] | 1-(4-((2R,3R)-1-(6- (3,3-difluoro-4-(4- methyl-1-(oxetan- 3-yl)-1H-pyrazol- 5-yl)piperidin-1- yl)-2- (trifluoromethyl) pyrimidin-4-yl)-2- methylazetidin-3- yl)piperazin-1- yl)prop-2-en-1-one | 2-009-1 | 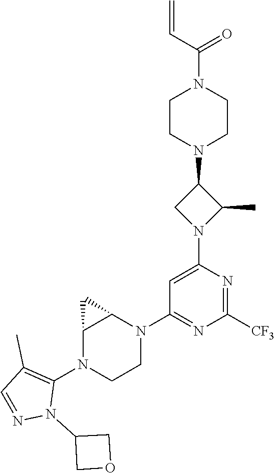 [SMILES: C=CC(=O)N1CCN([C@@H]2CN(c3cc(N4CCN(c5c(C)cnn5C5COC5)[C@@H]5C[C@@H]54)nc(C(F)(F)F)n3)[C@@H]2C)CC1] | 1-(4-((2R,3R)-2- methyl-1-(6- ((15,6R)-5-(4- methyl-1-(oxetan- 3-yl)-1H-pyrazol- 5-yl)-2,5- diazabicyclo[4.1.0] heptan-2-yl)-2- (trifluoromethyl) pyrimidin-4- yl)azetidin-3- yl)piperazin-1- yl)prop-2-en-1-one |
| 2-009-2 | 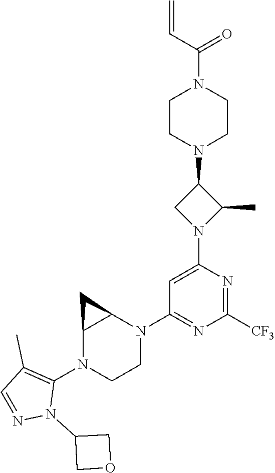 [SMILES: C=CC(=O)N1CCN([C@@H]2CN(c3cc(N4CCN(c5c(C)cnn5C5COC5)[C@H]5C[C@H]54)nc(C(F)(F)F)n3)[C@@H]2C)CC1] | 1-(4-((2R,3R)-2- methyl-1-(6- ((12,65)-5-(4- methyl-1-(oxetan- 3-yl)-1H-pyrazol- 5-yl)-2,5- diazabicyclo[4.1.0] heptan-2-yl)-2- (trifluoromethyl) pyrimidin-4- yl)azetidin-3- yl)piperazin-1- yl)prop-2-en-1-one | 2-013 | 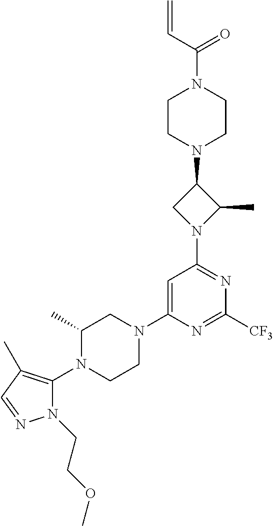 [SMILES: C=CC(=O)N1CCN([C@@H]2CN(c3cc(N4CCN(c5c(C)cnn5CCOC)[C@H](C)C4)nc(C(F)(F)F)n3)[C@@H]2C)CC1] | 1-(4-((2R,3R)-1-(6- ((R)-4-(1-(2- methoxyethyl)-4- methyl-1H- pyrazol-5-yl)-3- methylpiperazin-1- yl)-2- (trifluoromethyl) pyrimidin-4-yl)-2- methylazetidin-3- yl)piperazin-1- yl)prop-2-en-1-one |
| 2-010 | 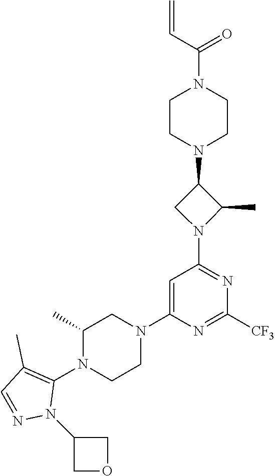 [SMILES: C=CC(=O)N1CCN([C@@H]2CN(c3cc(N4CCN(c5c(C)cnn5C5COC5)[C@H](C)C4)nc(C(F)(F)F)n3)[C@@H]2C)CC1] | 1-(4-((2R,3R)-2- methyl-1-(6-((R)-3- methyl-4-(4- methyl-1-(oxetan- 3-yl)-1H-pyrazol- 5-yl)piperazin-1- yl)-2- (trifluoromethyl) pyrimidin-4- yl)azetidin-3- yl)piperazin-1- yl)prop-2-en-1-one | 2-014 | 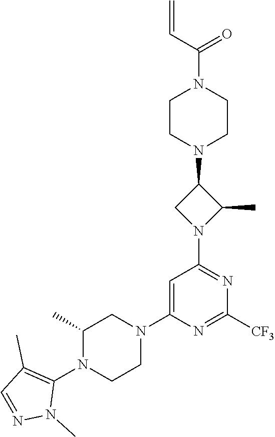 [SMILES: C=CC(=O)N1CCN([C@@H]2CN(c3cc(N4CCN(c5c(C)cnn5C)[C@H](C)C4)nc(C(F)(F)F)n3)[C@@H]2C)CC1] | 1-(4-((2R,3R)-1-(6- ((R)-4-(1,4- dimethyl-1H- pyrazol-5-yl)-3- methylpiperazin-1- yl)-2- (trifluoromethyl) pyrimidin-4-yl)-2- methylazetidin-3- yl)piperazin-1- yl)prop-2-en-1-one |
| 2-011 | 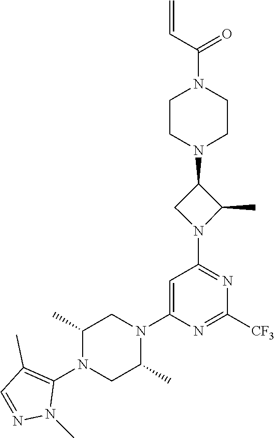 [SMILES: C=CC(=O)N1CCN([C@@H]2CN(c3cc(N4C[C@@H](C)N(c5c(C)cnn5C)C[C@H]4C)nc(C(F)(F)F)n3)[C@@H]2C)CC1] | 1-(4-((2R,3R)-1-(6- ((2R,5R)-4-(1,4- dimethyl-1H- pyrazol-5-yl)-2,5- dimethylpiperazin- 1-yl)-2- (trifluoromethyl) pyrimidin-4-yl)-2- methylazetidin-3- yl)piperazin-1- yl)prop-2-en-1-one | 2-015 | 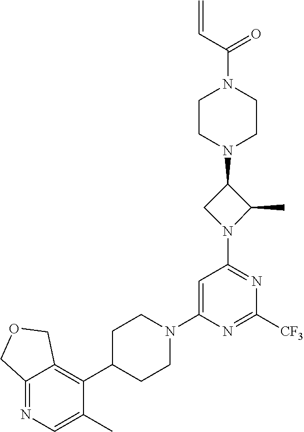 [SMILES: C=CC(=O)N1CCN([C@@H]2CN(c3cc(N4CCC(c5c(C)cnc6c5COC6)CC4)nc(C(F)(F)F)n3)[C@@H]2C)CC1] | 1-(4-((2.R.3R)-2- methyl-1-(6-(4-(3- methyl-5.7- dihydrofuro[3,4- b]pyridin-4- yl)piperidin-1-yl)- 2-(trifluoromethyl) pyrimidint-4- yl)azetidin-3- yl)piperazin-1- yl)prop-2-en-1-one |
| 2-012 | 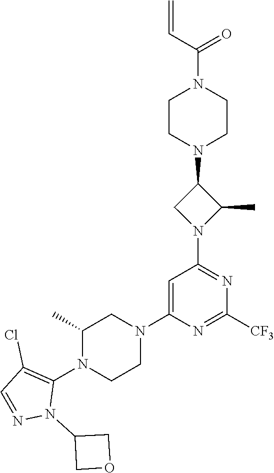 [SMILES: C=CC(=O)N1CCN([C@@H]2CN(c3cc(N4CCN(c5c(Cl)cnn5C5COC5)[C@H](C)C4)nc(C(F)(F)F)n3)[C@@H]2C)CC1] | 1-(4-((2R,3R)-1-(6- ((R)-4-(4-chloro-1- (oxetan-3-yl)-1H- pyrazol-5-yl)-3- methylpiperazin-1- yl)-2- (trifluoromethyl) pyrimidin-4-yl)-2- methylazetidin-3- yl)piperazin-1- yl)prop-2-en-1-one | 2-017 | 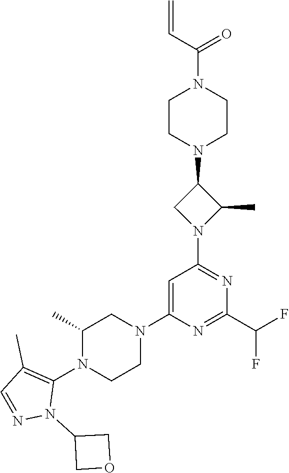 [SMILES: C=CC(=O)N1CCN([C@@H]2CN(c3cc(N4CCN(c5c(C)cnn5C5COC5)[C@H](C)C4)nc(C(F)F)n3)[C@@H]2C)CC1] | 1-(4-((2R,3R)-1-(2- (difluoromethyl)-6- ((R)-3-methyl-4-(4- methyl-1-(oxetan- 3-yl)-1.H-pyrazol- 5-yl)piperazin-1- yl)pyrimidin-4-yl)- 2-methylazetidin- 3-yl)piperazin-1- yl)prop-2-en-1-one |
| 2-018 | 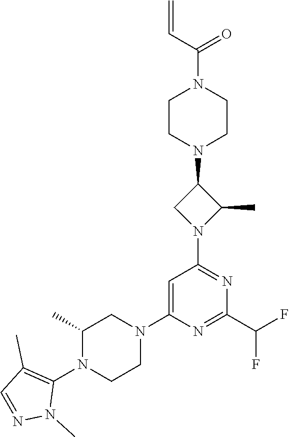 [SMILES: C=CC(=O)N1CCN([C@@H]2CN(c3cc(N4CCN(c5c(C)cnn5C)[C@H](C)C4)nc(C(F)F)n3)[C@@H]2C)CC1] | 1-(4-((2R,3R)-1-(2- (difluoromethyl)-6- ((R)-4-(1,4- dimethyl-1H- pyrazol-5-yl)-3- methylpiperazin-1- yl)pyrimidin-4-yl)- 2-methylazetidin- 3-yl)piperazin-1- yl)prop-2-en-1-one | 2-022 | 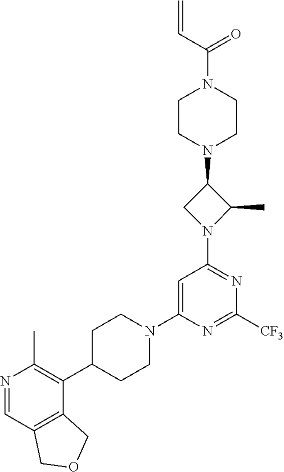 [SMILES: C=CC(=O)N1CCN([C@@H]2CN(c3cc(N4CCC(c5c(C)ncc6c5COC6)CC4)nc(C(F)(F)F)n3)[C@@H]2C)CC1] | 1-(4-((2R,3R)-2- methyl-1-(6-(4-(6- methyl-1.3- dihydrofuro[3,4- c]pyridin-7- yl)piperidin-1-yl)- 2- (trifluoromethyl) pyrimidin-4- yl)azetidin-3- yl)piperazin-1- yl)prop-2-en-1-one |
| 2-019 | 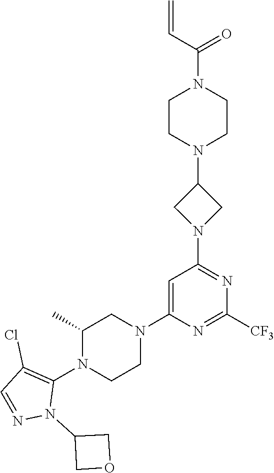 [SMILES: C=CC(=O)N1CCN(C2CN(c3cc(N4CCN(c5c(Cl)cnn5C5COC5)[C@H](C)C4)nc(C(F)(F)F)n3)C2)CC1] | (R)-1-(4-(1-(6-(4- (4-chloro-1- (oxetan-3-yl)-1H- pyrazol-5-yl)-3- methylpiperazin-1- yl)-2- (trifluoromethyl) pyrimidin-4- yl)azetidin-3- yl)piperazin-1- yl)prop-2-en-1-one | 2-023 | 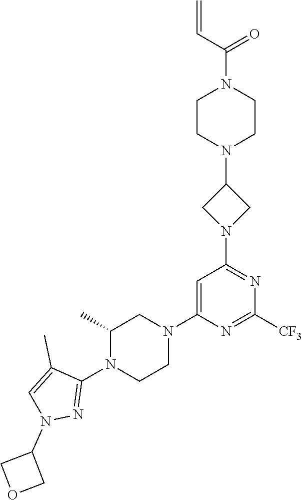 [SMILES: C=CC(=O)N1CCN(C2CN(c3cc(N4CCN(c5nn(C6COC6)cc5C)[C@H](C)C4)nc(C(F)(F)F)n3)C2)CC1] | (R)-1-(4-(1-(6-(3- methy-1-4-(4- methyl-1-(oxetan- 3-yl)-1H-pyrazol- 3-yl)piperazin-1- yl)-2- (trifluoromethyl) pyrimidin-4- yl)azetidin-3- yl)piperazin-1- yl)prop-2-en-1-one |
| 2-020 | 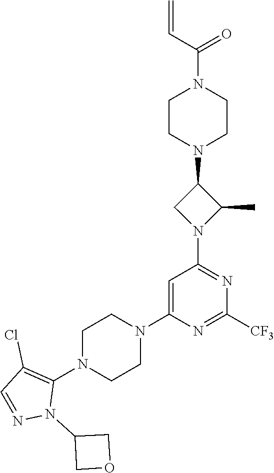 [SMILES: C=CC(=O)N1CCN([C@@H]2CN(c3cc(N4CCN(c5c(Cl)cnn5C5COC5)CC4)nc(C(F)(F)F)n3)[C@@H]2C)CC1] | 1-(4-((2R,3R)-1-(6- (4-(4-chloro-1- (oxetan-3-yl)-1H- pyrazol-5- yI)piperazin-1-yl)- 2-(trifluoromethyl) pyrimidin-4-yl)-2- methylazetidin-3- yl)piperazin-1- yl)prop-2-en-1-one | 2-024 | 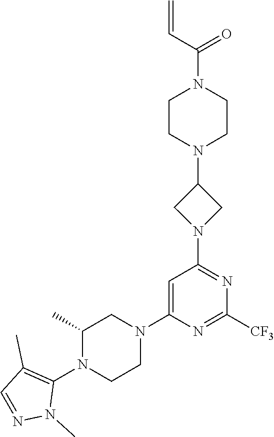 [SMILES: C=CC(=O)N1CCN(C2CN(c3cc(N4CCN(c5c(C)cnn5C)[C@H](C)C4)nc(C(F)(F)F)n3)C2)CC1] | (R)-1-(4-(1-(6-(4- (1,4-dimethyl-1H- pyrazol-5-yl)-3- methylpiperazin-1- yl-2- (trifluoromethyl) pyrimidin-4- yl)azetidin-3- yl)piperazin-1- yl)prop-2-en-1-one |
| 2-021 | 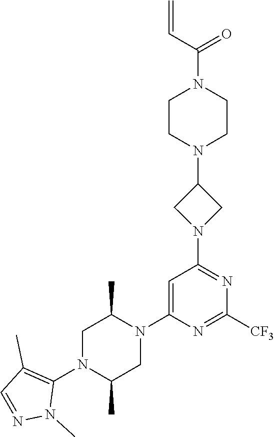 [SMILES: C=CC(=O)N1CCN(C2CN(c3cc(N4C[C@@H](C)N(c5c(C)cnn5C)C[C@H]4C)nc(C(F)(F)F)n3)C2)CC1] | 1-(4-(1-(6- ((2R,5R)-4-(1,4- dimethyl-1H- pyrazol-5-yl)-2,5- dinicthylpiperazin- 1-yl)-2- (trifluoromethyl) pyrimidin-1- yl)azetidin-3- yl)piperazin-1- yl)prop-2-en-1-one | 2-025 | 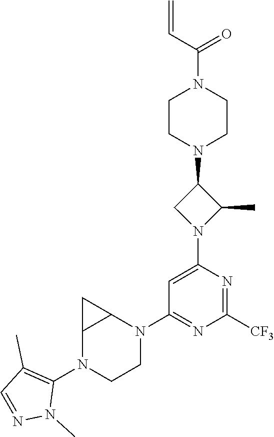 [SMILES: C=CC(=O)N1CCN([C@@H]2CN(c3cc(N4CCN(c5c(C)cnn5C)C5CC54)nc(C(F)(F)F)n3)[C@@H]2C)CC1] | 1-(4-((2R,3R)-1-(6- (5-(1.4-dimethy]- 1H-pyrazol-5-yl)- 2,5- diazabicyclo[4.1.0] heptan-2-yl)-2- (trifluoromethyl) pyrimidin-4-yl)-2- methylazetidin-3- yl)piperazin-1- yl)prop-2-en-1-one |
| 2-025-1 | 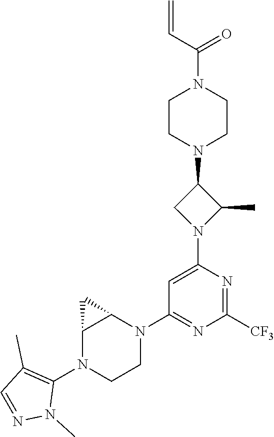 [SMILES: C=CC(=O)N1CCN([C@@H]2CN(c3cc(N4CCN(c5c(C)cnn5C)[C@@H]5C[C@@H]54)nc(C(F)(F)F)n3)[C@@H]2C)CC1] | 1-(4-((2R,3R)-1-(6- ((LS,6R)-5-(1,4- dimethyl-1H- pyrazol-5-yl)-2,5- diazabicyclo[4.1.0] heptan-2-yl)-2- (trifluoromethyl) pyrimidin-4-yl)-2- methylazetidin-3- yl)piperazin-1- yl)prop-2-en-1-one | 2-028 | 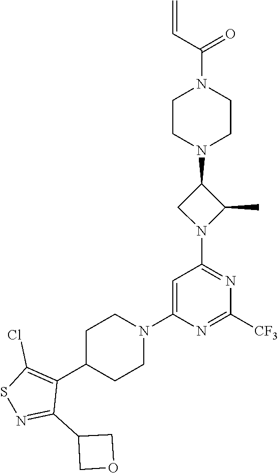 [SMILES: C=CC(=O)N1CCN([C@@H]2CN(c3cc(N4CCC(c5c(C6COC6)nsc5Cl)CC4)nc(C(F)(F)F)n3)[C@@H]2C)CC1] | 1-(4-((2R,3R)-1-(6- (4-(5-chloro-3- (oxetan-3- yl)isothiazol-4- yl)piperidin-1-yl)- 2- (trifluoromethyl) pyrimidin-4-yl)-2- methylazetidin-3- yl)piperazin-1- yl)prop-2-en-1-one |
| 2-025-2 | 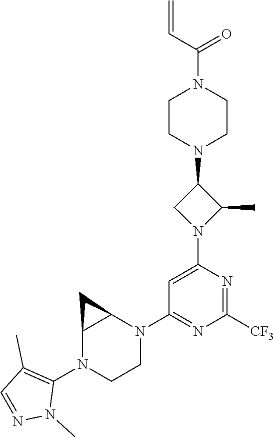 [SMILES: C=CC(=O)N1CCN([C@@H]2CN(c3cc(N4CCN(c5c(C)cnn5C)[C@H]5C[C@H]54)nc(C(F)(F)F)n3)[C@@H]2C)CC1] | 1-(4-((2R,3R)-1-(6- ((1R,6S)-5-(1,4- dimethyl-1H- pyrazol-5-yl)-2,5- diazabicyclo[4.1.0] heptan-2-yl)-2- (trifluoromethyl) pyrimidin-4-yl)-2- methylazetidin-3- yl)piperazin-1- yl)prop-2-en-1-one | 2-029 | 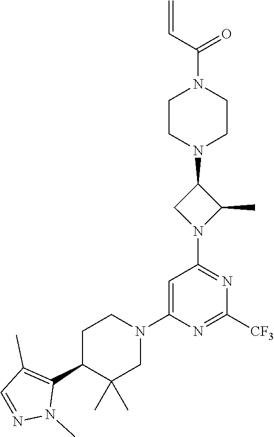 [SMILES: C=CC(=O)N1CCN([C@@H]2CN(c3cc(N4CC[C@H](c5c(C)cnn5C)C(C)(C)C4)nc(C(F)(F)F)n3)[C@@H]2C)CC1] | 1-(4-((2R,3R)-1-(6- ((S)-4-(1,4- dimethyl-1H- pyrazol-5-yl)-3,3- dimethylpiperidin- 1-yl)-2- (trifluoromethyl) pyrimidin-4-yl)-2- methylazetidin-3- yl)piperazin-1- yl)prop-2-en-1-one |
| 2-026 | 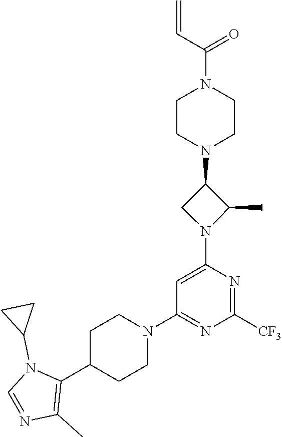 [SMILES: C=CC(=O)N1CCN([C@@H]2CN(c3cc(N4CCC(c5c(C)ncn5C5CC5)CC4)nc(C(F)(F)F)n3)[C@@H]2C)CC1] | 1-(4-((2R,3R)-1-(6- (4-(1-cyclopropyl- 4-methyl-1H- imidazol-5- yl)piperidin-1-yl)- 2- (trifluoromethyl) pyrimidin-4-yl)-2- methylazetidin-3- yl)piperazin-1- yl)prop-2-en-1-one | 2-030 | 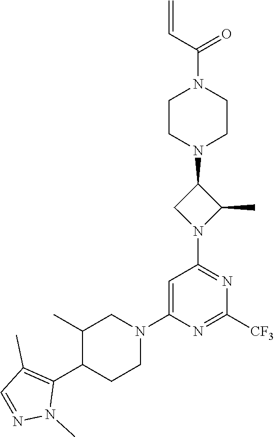 [SMILES: C=CC(=O)N1CCN([C@@H]2CN(c3cc(N4CCC(c5c(C)cnn5C)C(C)C4)nc(C(F)(F)F)n3)[C@@H]2C)CC1] | 1-(4-((2R,3R)-1-(6- (4-(1,4-dimethyl- 1H-pyrazol-5-yl)- 3-methylpiperidin- 1-yl)-2- (trifluoromethyl) pyrimidin-4-yl)-2- methylazetidin-3- yl)piperazin-1- yl)prop-2-en-1-one |
| 2-027 | 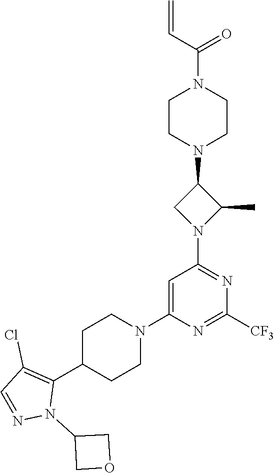 [SMILES: C=CC(=O)N1CCN([C@@H]2CN(c3cc(N4CCC(c5c(Cl)cnn5C5COC5)CC4)nc(C(F)(F)F)n3)[C@@H]2C)CC1] | 1-(4-((2R,3R)-1-(6- (4-(4-chloro-1- (oxetan-3-yl)-1H- pyrazol-5- yl)piperidin-1-yl)- 2- (trifluoromethyl) pyrimidin-4-yl)-2- methylazetidin-3- yl)piperazin-1- yl)prop-2-en-1-one | 2-030-1 | 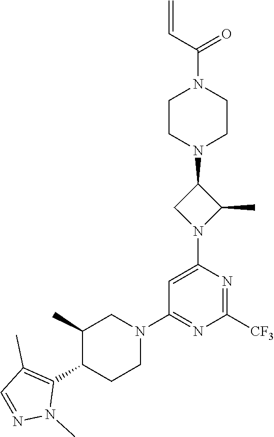 [SMILES: C=CC(=O)N1CCN([C@@H]2CN(c3cc(N4CC[C@H](c5c(C)cnn5C)[C@@H](C)C4)nc(C(F)(F)F)n3)[C@@H]2C)CC1] | 1-(4-((2R,3R)-1-(6- ((3R,4S)-4-(1,4- dimethyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one |
| 2-030-2 | 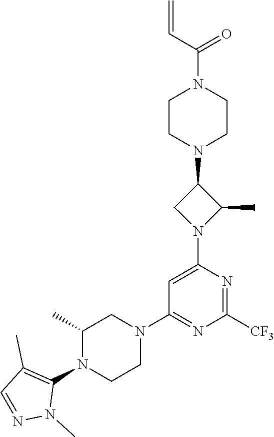 | 1-(4-((2R,3R)-1-(6- ((3.S,4R)-4-(1,4- dimethyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one | 2-032-2 | 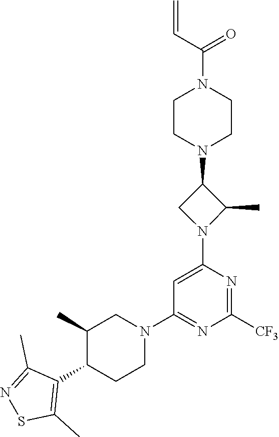 [SMILES: C=CC(=O)N1CCN([C@@H]2CN(c3cc(N4CC[C@H](c5c(C)nsc5C)[C@@H](C)C4)nc(C(F)(F)F)n3)[C@@H]2C)CC1] | 1-(4-((2R,3R)-1-(6- ((3R,4S)-4-(3,5- dimethylisothiazol- 4-yl)-3- methylpiperidin-1- yl)-2- (trifluoromethyl) pyrimidin-4-yl)-2- methylazetidin-3- yl)piperazin-1- yl)prop-2-en-1-one |
| 2-031 | 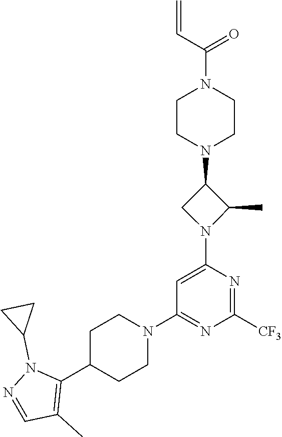 [SMILES: C=CC(=O)N1CCN([C@@H]2CN(c3cc(N4CCC(c5c(C)cnn5C5CC5)CC4)nc(C(F)(F)F)n3)[C@@H]2C)CC1] | 1-(4-((2R,3R)-1-(6- (4-(1-cyclopropyl- 4-methyl-1H- pyrazol-5- yl)piperidin-1-yl)- 2-(trifluoromethyl) pyrimidin-4-yl)-2- methylazetidin-3- yl)piperazin-1- yl)prop-2-en-1-one | 2-033 | 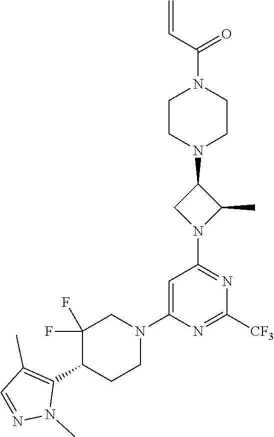 [SMILES: C=CC(=O)N1CCN([C@@H]2CN(c3cc(N4CC[C@H](c5c(C)cnn5C)C(F)(F)C4)nc(C(F)(F)F)n3)[C@@H]2C)CC1] | 1-(4-((2R,3R)-1-(6- ((R)-4-(1,4- dimethyl-1H- pyrazol-5-yl)-3,3- difluoropiperidin- 1-yl)-2- (trifluoromethyl) pyrimidin-4-yl)-2- methylazetidin-3- yl)piperazin-1- yl)prop-2-en-1-one |
| 2-032 | 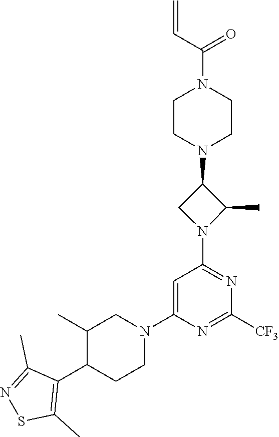 [SMILES: C=CC(=O)N1CCN([C@@H]2CN(c3cc(N4CCC(c5c(C)nsc5C)C(C)C4)nc(C(F)(F)F)n3)[C@@H]2C)CC1] | 1-(4-((2R,3R)-1-(6- (4-(3,5- dimethylisothiazol- 4-yl)-3- methylpiperidin-1- yl)-2- (trifluoromethyl) pyrimidin-4-yl)-2- methylazetidin-3- yl)piperazin-1- yl)prop-2-en-1-one | 2-034 | 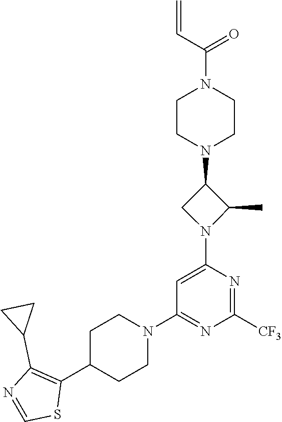 [SMILES: C=CC(=O)N1CCN([C@@H]2CN(c3cc(N4CCC(c5scnc5C5CC5)CC4)nc(C(F)(F)F)n3)[C@@H]2C)CC1] | 1-(4-((2R,3R)-1-(6- (4-(4- cyclopropylthiazol- 5-yl)piperidin-1- yl)-2- (trifluoromethyl) pyrimidin-4-yl)-2- methylazetidin-3- yl)piperazin-1- yl)prop-2-en-1-one |
| 2-032-1 | 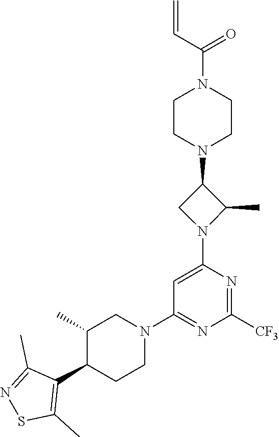 [SMILES: C=CC(=O)N1CCN([C@@H]2CN(c3cc(N4CC[C@@H](c5c(C)nsc5C)[C@H](C)C4)nc(C(F)(F)F)n3)[C@@H]2C)CC1] | 1-(4-((2R,3R)-1-(6- ((3S.4R)-4-(3.5- dimethylisothiazol- 4-yl)-3- methylpiperidin-1- yl)-2- (trifluoromethyl) pyrimidin-4-yl)-2- methylazetidin-3- yl)piperazin-1- yl)prop-2-en-1-one | 2-035 | 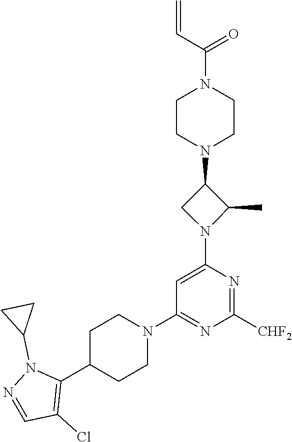 [SMILES: C=CC(=O)N1CCN([C@@H]2CN(c3cc(N4CCC(c5c(Cl)cnn5C5CC5)CC4)nc(C(F)F)n3)[C@@H]2C)CC1] | 1-(4-((2R,3R)-1-(6- (4-(4-chloro-1- (oxetan-3-yl)-1H- pyrazol-5- yl)piperidin-1-yl)- 2- (difluoromethyl) pyrimidin-4-yl)-2- methylazetidin-3- yl)piperazin-1- yl)prop-2-en-1-one |
| 2-036 | 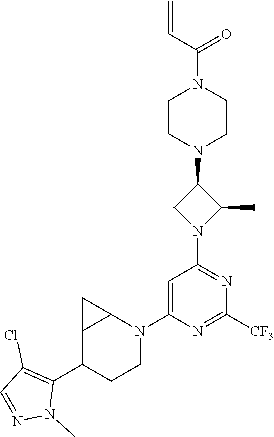 [SMILES: C=CC(=O)N1CCN([C@@H]2CN(c3cc(N4CCC(c5c(Cl)cnn5C)C5CC54)nc(C(F)(F)F)n3)[C@@H]2C)CC1] | 1-(4-((2R,3R)-1-(6- (5-(4-chloro-1- methyl-1H- pyrazol-5-yl)-2- azabicyclo[4.1.0] heptan-2-yl)-2- (trifluoromethyl) pyrimidin-4-yl)-2- methylazetidin-3- yl)piperazin-1- yl)prop-2-en-1-one | 2-038 | 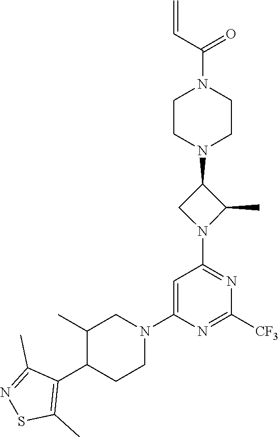 [SMILES: C=CC(=O)N1CCN([C@@H]2CN(c3cc(N4CCC(c5c(C)nsc5C)C(C)C4)nc(C(F)(F)F)n3)[C@@H]2C)CC1] | 1-(4-((2R,3R)-1-(6- (4-(3.5- dimethylisothiazol- 4-yl)-3- methylpiperidin-1- yl)-2- (trifluoromethyl) pyrimidin-4-yl)-2- methylazetidin-3- yl)piperazin-1- yl)prop-2-en-1-one |
| 2-036-1 | 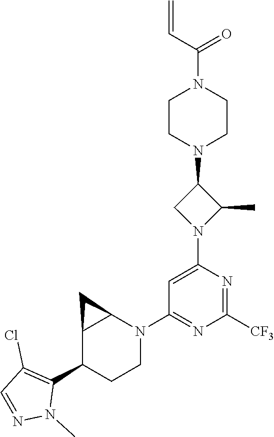 [SMILES: C=CC(=O)N1CCN([C@@H]2CN(c3cc(N4CC[C@@H](c5c(Cl)cnn5C)[C@H]5C[C@H]54)nc(C(F)(F)F)n3)[C@@H]2C)CC1] | 1-(4-((2R,3R)-1-(6- ((1R,5R,6R)-5-(4- chloro-1-methyl- 1H-pyrazol-5-yl)- 2- azabicyclo[4.1.0] heptan-2-yl)-2- (trifluoromethyl) pyrimidin-4-yl)-2- methylazetidin-3- yl)piperazin-1- yl)prop-2-en-1-one | 2-038-1 | 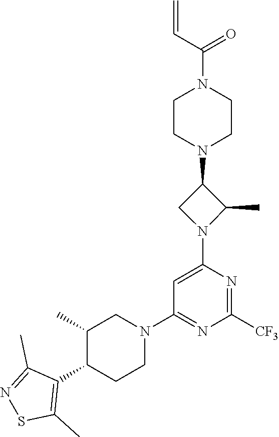 [SMILES: C=CC(=O)N1CCN([C@@H]2CN(c3cc(N4CC[C@H](c5c(C)nsc5C)[C@H](C)C4)nc(C(F)(F)F)n3)[C@@H]2C)CC1] | 1-(4-((2R,3R)-1-(6- ((35,45)-4-(3,5- dimethylisothiazol- 4-yl)-3- methylpiperidin-1- yl)-2- (trifluoromethyl) pyrimidin-4-yl)-2- methylazetidin-3- yl)piperazin-1- yl)prop-2-en-1-one |
| 2-036-2 | 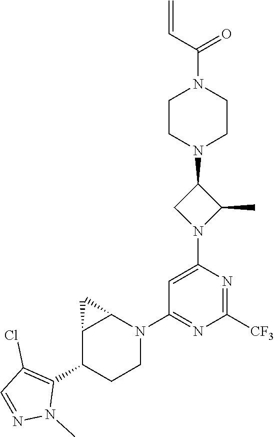 [SMILES: C=CC(=O)N1CCN([C@@H]2CN(c3cc(N4CC[C@H](c5c(Cl)cnn5C)[C@@H]5C[C@@H]54)nc(C(F)(F)F)n3)[C@@H]2C)CC1] | 1-(4-((2R,3R)-1-(6- ((1S,5S,6S)-5-(4- chloro-1-methyl- 1H-pyrazol-5-yl)- 2- azabicyclo[4.1.0] heptan-2-yl)-2- (trifluoromethyl) pyrimidin-4-yl)-2- methylazetidin-3- yl)piperazin-1- yl)prop-2-en-1-one | 2-038-2 | 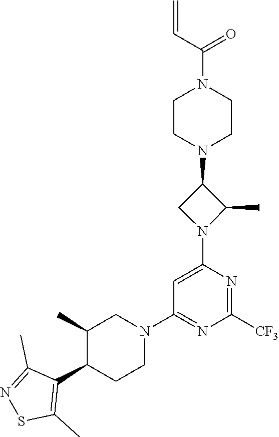 [SMILES: C=CC(=O)N1CCN([C@@H]2CN(c3cc(N4CC[C@@H](c5c(C)nsc5C)[C@@H](C)C4)nc(C(F)(F)F)n3)[C@@H]2C)CC1] | 1-(4-((2R,3R)-1-(6- ((3R.4R)-4-(3,5- dimethylisothiazol- 4-yl)-3- methylpiperidin-1- yl)-2- (trifluoromethyl) pyrimidin-4-yl)-2- methylazetidin-3- yl)piperaziu-1- yl)prop-2-en-1-one |
| 2-037 | 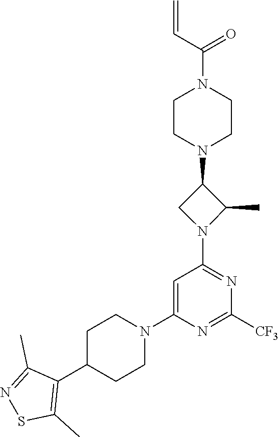 [SMILES: C=CC(=O)N1CCN([C@@H]2CN(c3cc(N4CCC(c5c(C)nsc5C)CC4)nc(C(F)(F)F)n3)[C@@H]2C)CC1] | 1-(4-((2R,3R)-1-(6- (4-(3,5- dimethylisothiazol- 4-yl)piperidin-1- yl)-2- (trifluoromethyl) pyrimidin-4-yl)-2- methylazetidin-3- yl)piperazin-1- yl)prop-2-en-1-one | 2-039 | 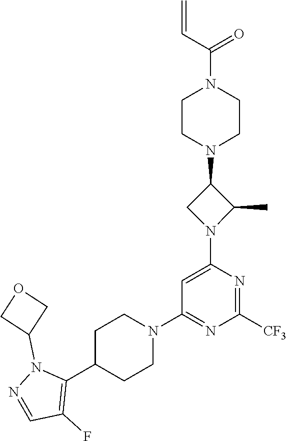 [SMILES: C=CC(=O)N1CCN([C@@H]2CN(c3cc(N4CCC(c5c(F)cnn5C5COC5)CC4)nc(C(F)(F)F)n3)[C@@H]2C)CC1] | 1-(4-((2R,3R)-1-(6- (4-(4-fluoro-1- (oxetan-3-yl)-1H- pyrazol-5- yl)piperidin-1-yl)- 2- (trifluoromethyl) pyrimidin-4-yl)-2- methylazetidin-3- yl)piperazin-1- yl)prop-2-en-1-one |
| 2-040 | 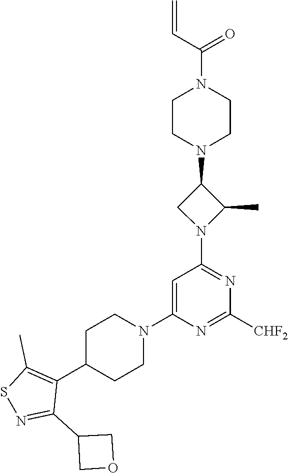 [SMILES: C=CC(=O)N1CCN([C@@H]2CN(c3cc(N4CCC(c5c(C6COC6)nsc5C)CC4)nc(C(F)F)n3)[C@@H]2C)CC1] | 1-(4-((2R,3R)-1-(2- (difluoromethyl)-6- (4-(5-methyl-3- (oxetan-3- yl)isothiazol-4- yl)piperidin-1- yl)pyrimidin-4-yl)- 2-methylazetidin- 3-yl)piperazin-1- yl)prop-2-en-1-one | 2-042-2 | 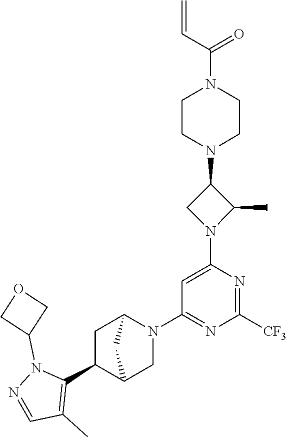 | 1-(4-((2R,3R)-2- methyl-1-(6- ((1S,4R,5S)-5-(4- methyl-1-(oxetan- 3-yl)-1H-pyrazol- 5-yl)-2- azabicyclo[2.2.1] heptan-2-yl)-2- (trifluoromethyl) pyrimidin-4- ylazetidin-3- yl)piperazin-1- yl)prop-2-en-1-one |
| 2-041 | 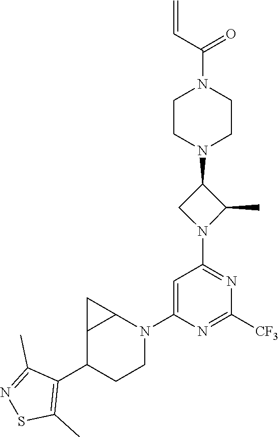 [SMILES: C=CC(=O)N1CCN([C@@H]2CN(c3cc(N4CCC(c5c(C)nsc5C)C5CC54)nc(C(F)(F)F)n3)[C@@H]2C)CC1] | 1-(4-((2R,3R)-1-(6- (5-(3.5- dimethylisothiazol- 4-yl)-2- azabicyclo[4.1.0] heptan-2-yl)-2- (trifluoromethyl) pyrimidin-4-yl)-2- methylazetidin-3- yl)piperazin-1- yl)prop-2-en-1-one | 2-043 | 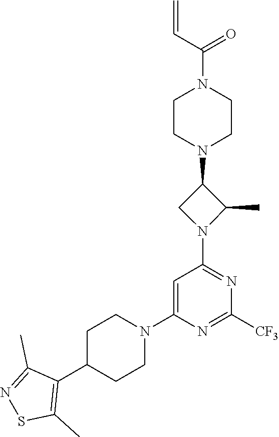 [SMILES: C=CC(=O)N1CCN([C@@H]2CN(c3cc(N4CCC(c5c(C)nsc5C)CC4)nc(C(F)(F)F)n3)[C@@H]2C)CC1] | 1-(4-((2R,3R)-1-(6- (4-(3,5- dimethylisothiazol- 4-yl)piperidin-1- yl)-2- (trifluoromethyl) pyrimidin-4-yl)-2- methylazetidin-3- yl)ypiperazin-1- yl)prop-2-en-1-one |
| 2-042 | 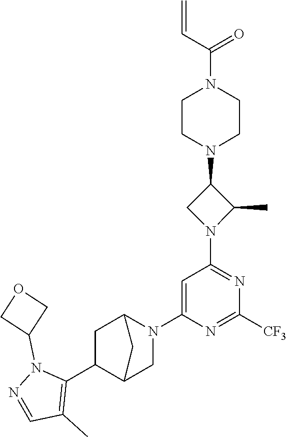 [SMILES: C=CC(=O)N1CCN([C@@H]2CN(c3cc(N4CC5CC4CC5c4c(C)cnn4C4COC4)nc(C(F)(F)F)n3)[C@@H]2C)CC1] | 1-(4-((2R,3R)-2- methyl-1-(6-(5-(4- methyl-1-(oxetan- 3-yl)-1H-pyrazol- 5-yl)-2- azabicyclo[2.2.1] heptan-2-yl)-2- (trifluoromethyl) pyrimidin-4- yl)azetidin-3- yl)piperazin-1- yl)prop-2-en-1-one | 2-044 | 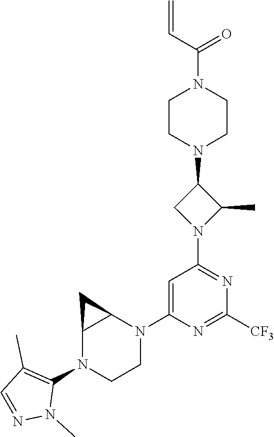 | 1-(4-((2R,3R)-1-(6- ((1R,5R,6R)-5-(1,4- dimethyl-1H- pyrazol-5-yl)-2- azabicyclo[4.1.0] heptan-2-yl)-2- (trifluoromethyl) pyrimidin-4-yl)-2- methylazetidin-3- yl)piperaziu-1- yl)prop-2-en-1-one |
| 2-042-1 | 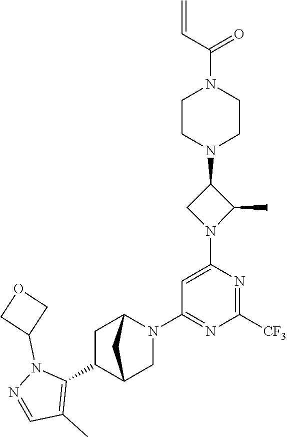 | 1-(4-((2R,3R)-2- methy1-1-(6- ((IR,45,5R)-5-(4- methyl-1-(oxetan- 3-yl)-1H-pyrazol- 5-yl)-2- azabicyclo[2.2.1] heptan-2-yl)-2- (trifluoromethyl) pyrimidin-4- yl)azetidin-3- yl)piperazin-1- yl)prop-2-en-1-one | 2-045 | 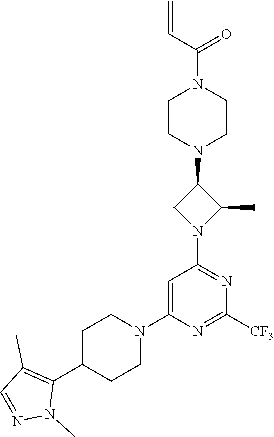 [SMILES: C=CC(=O)N1CCN([C@@H]2CN(c3cc(N4CCC(c5c(C)cnn5C)CC4)nc(C(F)(F)F)n3)[C@@H]2C)CC1] | 1-(4-((2R,3R)-1-(6- (4-(1,4-dimethyl- LA-pyrazol-5- yl)piperidin-1-yl)- 2-(trifluoromethyl) pyrimidin-4-yl)-2- methylazetidin-3- yl)piperazin-\|- yl)prop-2-en-1-one |
| 2-046 | 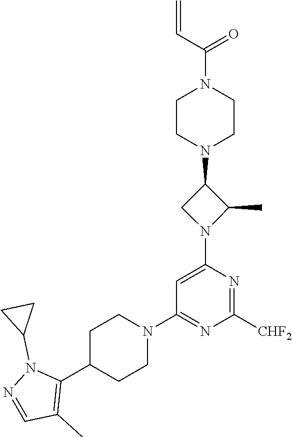 [SMILES: C=CC(=O)N1CCN([C@@H]2CN(c3cc(N4CCC(c5c(C)cnn5C5CC5)CC4)nc(C(F)F)n3)[C@@H]2C)CC1] | 1-(4-((2R,3R)-1-(6- (4-(1-cyclopropyl- 4-methyl-1H- pyrazol-5- yl)piperidin-1-yl)- 2-(difluoromethyl) pyrimidin-4-yl)-2- methylazetidin-3- yl)piperazin-1- yl)prop-2-en-1-one | 2-050 | 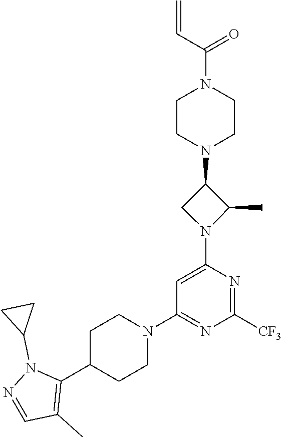 [SMILES: C=CC(=O)N1CCN([C@@H]2CN(c3cc(N4CCC(c5c(C)cnn5C5CC5)CC4)nc(C(F)(F)F)n3)[C@@H]2C)CC1] | 1-(4-((2R.3R)-1-(6- (4-(1-cyclopropyl- 4-methyl-1H- pyrazol-5- yl)piperidin-1-yl)- 2-(trifluoromethyl) pyrimidin-4-yl)-2- methylazetidin-3- yl)piperazin-1- yl)prop-2-en-1-one |
| 2-047 | 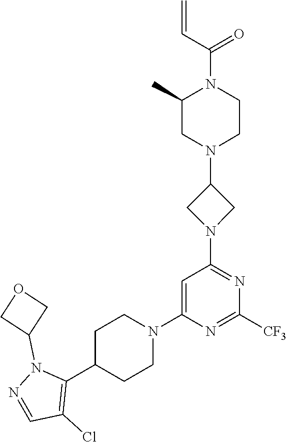 [SMILES: C=CC(=O)N1CCN(C2CN(c3cc(N4CCC(c5c(Cl)cnn5C5COC5)CC4)nc(C(F)(F)F)n3)C2)C[C@H]1C] | (R)-1-(4-(1-(6-(4- (4-chloro-1- (oxetan-3-yl)-1H- pyrazol-5- yl)piperidin-1-yl)- 2-(trifluoromethyl) pyrimidin-4- yl)azetidin-3-yl)-2- methylpiperazin-1- yl)prop-2-en-1-one | 2-051 | 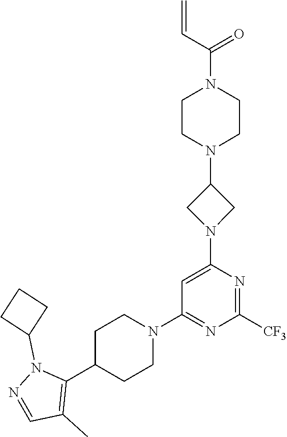 [SMILES: C=CC(=O)N1CCN(C2CN(c3cc(N4CCC(c5c(C)cnn5C5CCC5)CC4)nc(C(F)(F)F)n3)C2)CC1] | 1-(4-(1-(6-(4-(1- cyclobutyl-4- methyl-1H- pyrazol-5- yl)piperidin-1-yl)- 2-(trifluoromethyl) pyrimnidin-4- yl)azetidin-3- yl)piperazin-1- yl)prop-2-en-1-one |
| 2-048 | 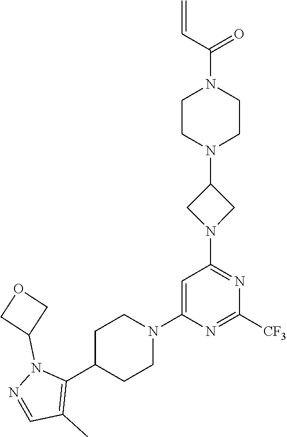 [SMILES: C=CC(=O)N1CCN(C2CN(c3cc(N4CCC(c5c(C)cnn5C5COC5)CC4)nc(C(F)(F)F)n3)C2)CC1] | 1-(4-(1-(6-(4-(4- methyl-1-(oxetan- 3-yl)-1H-pyrazol- 5-yl)piperidin-1- yl)-2- (trifluoromethyl) pyrimidin-4- yl)azetidin-3- yl)piperazin-1- yl)prop-2-en-1-one | 2-052 | 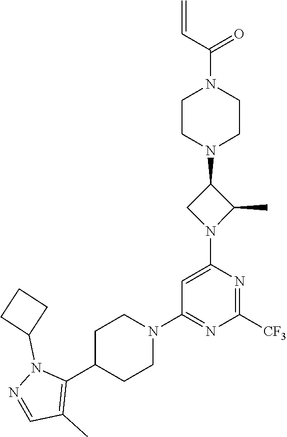 [SMILES: C=CC(=O)N1CCN([C@@H]2CN(c3cc(N4CCC(c5c(C)cnn5C5CCC5)CC4)nc(C(F)(F)F)n3)[C@@H]2C)CC1] | 1-(4-((2R,3R)-1-(6- (4-(1-cyclobutyl-4- methyl-1H- pyrazol-5- yl)piperidin-1-yl)- 2-(trifluoromethyl) pyrimidin-4-yl)-2- methylazetidin-3- yl)piperaziu-1- yl)prop-2-en-1-one |
| 2-049 | 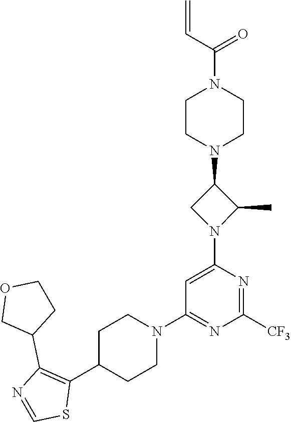 [SMILES: C=CC(=O)N1CCN([C@@H]2CN(c3cc(N4CCC(c5scnc5C5CCOC5)CC4)nc(C(F)(F)F)n3)[C@@H]2C)CC1] | 1-(4-((2R,3R)-2- methyl-1-(6-(4-(4- (tetrahydrofuran-3- yl)thiazol-5- yl)piperidin-1-yl)- 2-(trifluoromethyl) pyrimidin-1- yl)azetidin-3- yl)piperazin-1- yl)prop-2-en-1-one | 2-053 | 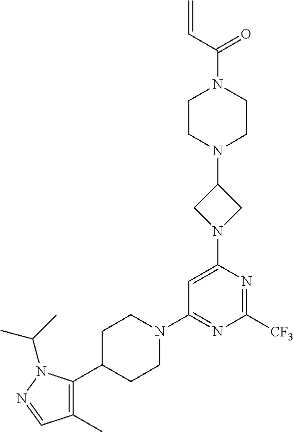 [SMILES: C=CC(=O)N1CCN(C2CN(c3cc(N4CCC(c5c(C)cnn5C(C)C)CC4)nc(C(F)(F)F)n3)C2)CC1] | 1-(4-(1-(6-(4-(1- isopropyl-4- methyl-1H- pyrazol-5- yl)piperidin-1-yl)- 2-(trifluoromethyl) pyrimidin-4- yl)azetidin-3- ylpiperazin-1- yl)prop-2-en-1-one |
| 2-054 | 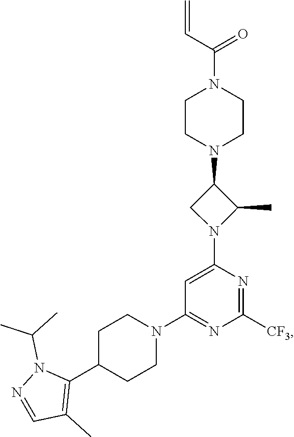 [SMILES: C=CC(=O)N1CCN([C@@H]2CN(c3cc(N4CCC(c5c(C)cnn5C(C)C)CC4)nc(C(F)(F)F)n3)[C@@H]2C)CC1] | 1-(4-((2R,3R)-1-(6- (4-(1-isopropyl-4- methyl-1H- pyrazol-5- yl)piperidin-1-yl)- 2-(trifluoromethyl) pyrimidin-4-yl)-2- methylazetidin-3- yl)piperazin-1- yl)prop-2-en-1-one | 2-058 | 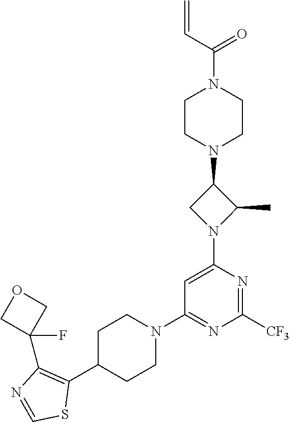 [SMILES: C=CC(=O)N1CCN([C@@H]2CN(c3cc(N4CCC(c5scnc5C5(F)COC5)CC4)nc(C(F)(F)F)n3)[C@@H]2C)CC1] | 1-(4-((2R.3R)-1-(6- (4-(4-(3- fluorooxetan-3- yl)thiazol-5- yl)piperidin-1-yl)- 2-(trifluoromethyl) pyrimidin-4-yl)-2- methylazetidin-3- yl)piperazin-1- yl)prop-2-en-1-one |
| 2-055 | 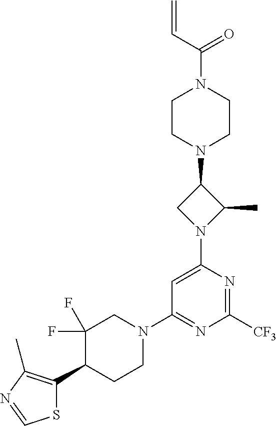 [SMILES: C=CC(=O)N1CCN([C@@H]2CN(c3cc(N4CC[C@@H](c5scnc5C)C(F)(F)C4)nc(C(F)(F)F)n3)[C@@H]2C)CC1] | 1-(4-((2R,3R)-1-(6- ((R)-3,3-difluoro-4- (4-methylthiazol-5- yl)piperidin-1-yl)- 2-(trifluoromethyl) pyrimidin-4-yl)-2- methylazetidin-3- yl)piperazin-1- yl)prop-2-en-1-one | 2-059 | 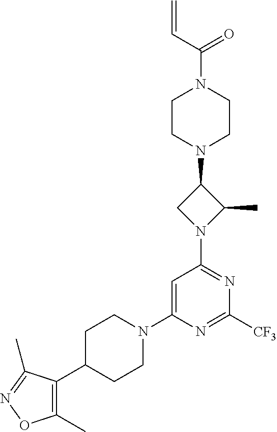 [SMILES: C=CC(=O)N1CCN([C@@H]2CN(c3cc(N4CCC(c5c(C)noc5C)CC4)nc(C(F)(F)F)n3)[C@@H]2C)CC1] | 1-(4-((2R,3R)-1-(6- (4-(3,5- dimethylisoxazol- 4-yl)piperidin-1- yl)-2- (trifluoromethyl) pyrimidin-4-yl)-2- methylazetidin-3. yl)piperazin-1- yl)prop-2-en-1-one |
| 2-056 | 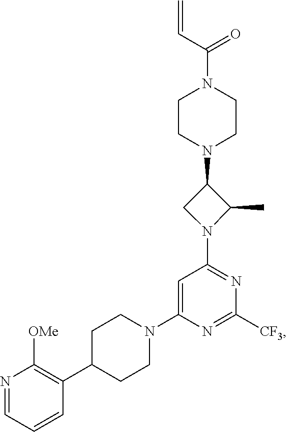 [SMILES: C=CC(=O)N1CCN([C@@H]2CN(c3cc(N4CCC(c5cccnc5OC)CC4)nc(C(F)(F)F)n3)[C@@H]2C)CC1] | 1-(4-((2R,3R)-1-(6- (4-(2- methoxypyridin-3- yl)piperidm-1-yl)- 2-(trifluoromethyl) pyrimidin-4-yl)-2- methylazetidin-3- yl)piperazin-1- yl)prop-2-en-1-one | 2-060 | 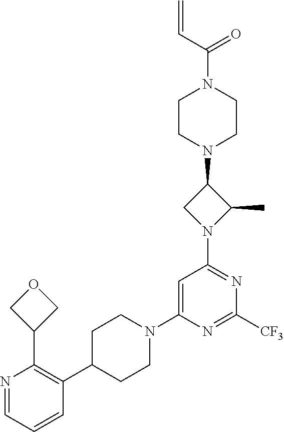 [SMILES: C=CC(=O)N1CCN([C@@H]2CN(c3cc(N4CCC(c5cccnc5C5COC5)CC4)nc(C(F)(F)F)n3)[C@@H]2C)CC1] | 1-(4-((2R,3R)-2- methyl-1-(6-(4-(2- (oxetan-3- yl)pyridin-3- yl)piperidin-1-yl)- 2-(trifluoromethyl) pyrimidin-4- yl)azetidin-3- yl)piperaziu-1- yl)prop-2-en-1-one |
| 2-057 | 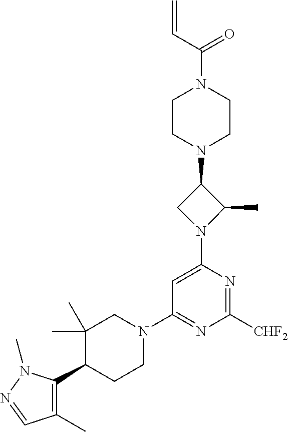 [SMILES: C=CC(=O)N1CCN([C@@H]2CN(c3cc(N4CC[C@@H](c5c(C)cnn5C)C(C)(C)C4)nc(C(F)F)n3)[C@@H]2C)CC1] | 1-(4-((2R,3R)-1-(2- (difluoromethyl)-6- ((S)-4-(1,4- dimethyl-1H- pyrazol-5-yl)-3,3- dimethylpiperidin- 1-yl)pyrimidin-4- yl)-2- methylazetidin-3- yl)piperazin-1- yl)prop-2-en-1-one | 2-061 | 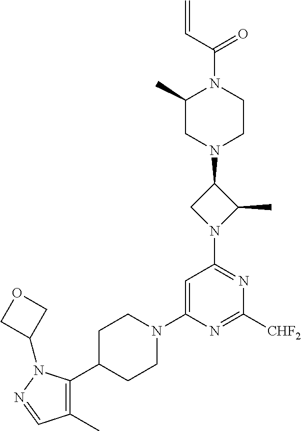 [SMILES: C=CC(=O)N1CCN([C@@H]2CN(c3cc(N4CCC(c5c(C)cnn5C5COC5)CC4)nc(C(F)F)n3)[C@@H]2C)C[C@H]1C] | 1-((R)-4-((2R,3R)- 1-(2- (difluoromethyl)-6- (4-(4-methyl-1- (oxetan-3-yl)-1H- pyrazol-5- yl)piperidin-1- yl)pyrimidin-4-yl)- 2-methylazetidin- 3-yl)-2- methylpiperazin-1- yl)prop-2-en-1-one |
| 2-062 | 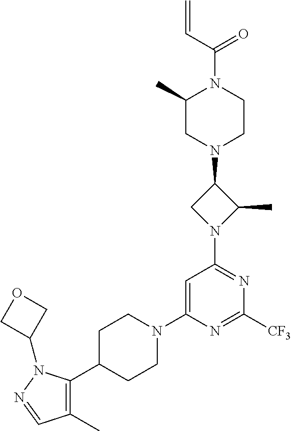 [SMILES: C=CC(=O)N1CCN([C@@H]2CN(c3cc(N4CCC(c5c(C)cnn5C5COC5)CC4)nc(C(F)(F)F)n3)[C@@H]2C)C[C@H]1C] | 1-((R)-2-methyl-4- ((2R,3R)-2-methyl- 1-(6-(4-(4-methyl- 1-(oxetan-3-y])- 1H-pyrazol-5- yl)piperidin-1-yl)- 2-(trifluoromethyl) pyrimidin-4- yl)azetidin-3- yl)piperazin-1- yl)prop-2-en-1-one | 2-067 | 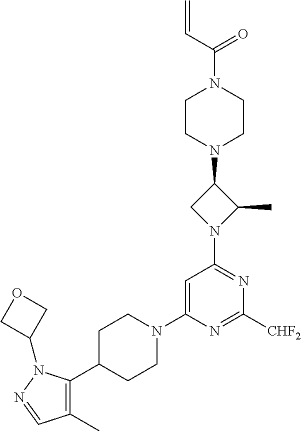 [SMILES: C=CC(=O)N1CCN([C@@H]2CN(c3cc(N4CCC(c5c(C)cnn5C5COC5)CC4)nc(C(F)F)n3)[C@@H]2C)CC1] | 1-(4-((2R,3R)-1-(2- (difluoromethyl)-6- (4-(4-methyl-1- (oxetan-3-yl)-1H- pyrazol-5- yl)piperidin-1- yl)pyrimidin-4-yl)- 2-methylazetidin- 3-yl)piperazin-1- yl)prop-2-en-1-one |
| 2-063 | 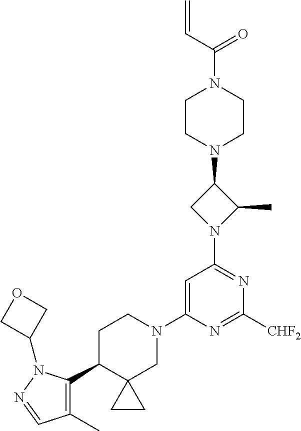 [SMILES: C=CC(=O)N1CCN([C@@H]2CN(c3cc(N4CC[C@H](c5c(C)cnn5C5COC5)C5(CC5)C4)nc(C(F)F)n3)[C@@H]2C)CC1] | 1-(4-((2R,3R)-1-(2- (difluoromethyl)-6- ((S)-8-(4-methyl-1- (oxetan-3-yl)-1H- pyrazol-5-yl)-5- azaspiro[2.5]octan- 5-yl)pyrimidin-4- yl)-2- methylazetidin-3- yl)piperazin-1- yl)prop-2-en-1-one | 2-068 | 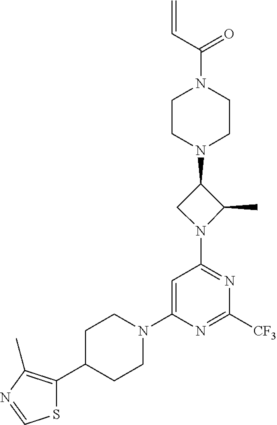 [SMILES: C=CC(=O)N1CCN([C@@H]2CN(c3cc(N4CCC(c5scnc5C)CC4)nc(C(F)(F)F)n3)[C@@H]2C)CC1] | 1-(4-((2R,3R)-2- methyl-1-(6-(4-(4- methylthiazol-5- yl)piperidin-1-yl)- 2-(trifluoromethyl) pyrimidin-4- yl)azetidin-3- yl)piperazin-1- yl)prop-2-en-1-one |
| 2-065 | 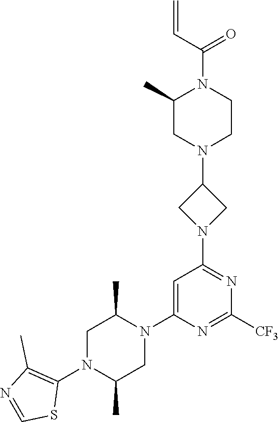 [SMILES: C=CC(=O)N1CCN(C2CN(c3cc(N4C[C@@H](C)N(c5scnc5C)C[C@H]4C)nc(C(F)(F)F)n3)C2)C[C@H]1C] | 1-((R)-4-(1-(6- ((2R,SR)-2,5- dimethy-1-4-(4- methylthiazol-5- yl)piperazin-1-yl)- 2-(trifluoromethyl) pyrimidin-4- yl)azetidin-3-yl)-2- methylpiperazin-1- yl)prop-2-en-1-one | 2-069 | 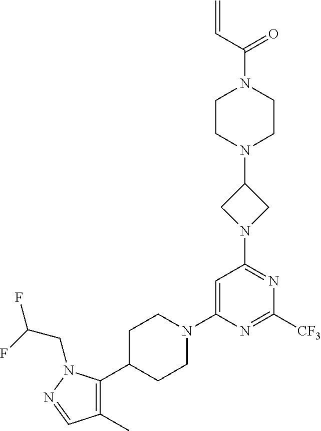 [SMILES: C=CC(=O)N1CCN(C2CN(c3cc(N4CCC(c5c(C)cnn5CC(F)F)CC4)nc(C(F)(F)F)n3)C2)CC1] | (2,2-difluoroethyl)- 4-methyl-1H- pyrazol-5- yl)piperidin-1-yl)- 2-(trifluoromethyl) pyrimidin-4- ylazetidin-3- yl)piperazin-1- yl)prop-2-en-1-one |
| 2-066 | 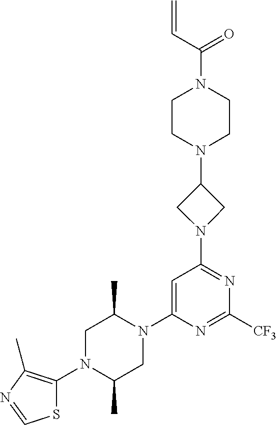 [SMILES: C=CC(=O)N1CCN(C2CN(c3cc(N4C[C@@H](C)N(c5scnc5C)C[C@H]4C)nc(C(F)(F)F)n3)C2)CC1] | 1-(4-(1-(6- ((2R,5R)-2,5- dimethyl-4-(4- methylthiazol-5- yl)piperazin-1-yl)- 2-(trifluoromethyl) pyrimidin-1- yl)azetidin-3- yl)piperazin-1- yl)prop-2-en-1-one | 2-070 | 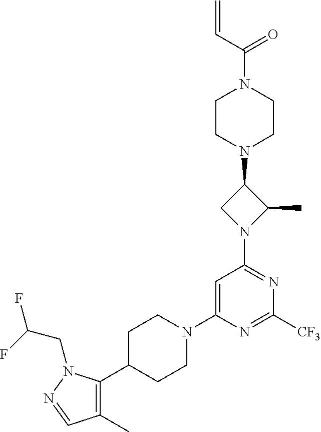 [SMILES: C=CC(=O)N1CCN([C@@H]2CN(c3cc(N4CCC(c5c(C)cnn5CC(F)F)CC4)nc(C(F)(F)F)n3)[C@@H]2C)CC1] | 1-(4-((2R,3R)-1-(6- (4-(1-(2,2- difluoroethyl)-4- methyl-1H- pyrazol-5- yl)piperidin-1-yl)- 2-(trifluoromethyl) pyrimidin-4-yl)-2- methylazetidin-3- yl)piperazin-1- yl)prop-2-en-1-one |
| 2-071 | 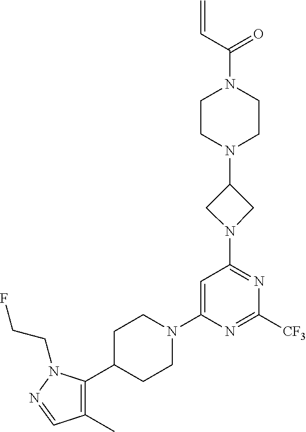 [SMILES: C=CC(=O)N1CCN(C2CN(c3cc(N4CCC(c5c(C)cnn5CCF)CC4)nc(C(F)(F)F)n3)C2)CC1] | 1-(4-(1-(6-(4-(1-(2- fluoroethyl)-4- methyl-1H- pyrazol-5- yl)piperidin-1-yl)- 2-(trifluoromethyl) pyrimidin-4- yl)azetidin-3- yl)piperazin-1- yl)prop-2-en-1-one | 2-075 | 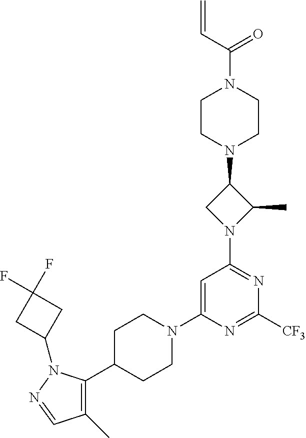 [SMILES: C=CC(=O)N1CCN([C@@H]2CN(c3cc(N4CCC(c5c(C)cnn5C5CC(F)(F)C5)CC4)nc(C(F)(F)F)n3)[C@@H]2C)CC1] | 1-(4-((2R,3R)-1-(6- (4-(1-(3,3- difluorocyclobutyl)- 4-methyl-1H- pyrazol-5- yl)piperidin-1-yl)- 2-(trifluoromethyl) pyrimidin-4-yl)-2- methylazetidin-3- yl)piperazin-1- yl)prop-2-en-1-one |
| 2-072 | 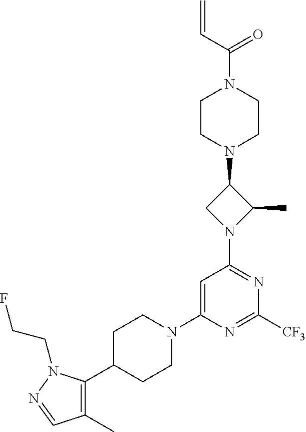 [SMILES: C=CC(=O)N1CCN([C@@H]2CN(c3cc(N4CCC(c5c(C)cnn5CCF)CC4)nc(C(F)(F)F)n3)[C@@H]2C)CC1] | 1-(4-((2R,3R)-1-(6- (4-(1-(2- fluoroethyl)-4- methyl-1H- pyrazol-5- yl)piperidin-1-yl)- 2-(trifluoromethyl) pyrimidin-4-yl)-2- methylazetidin-3- yl)piperazin-1- yl)prop-2-en-1-one | 2-076 | 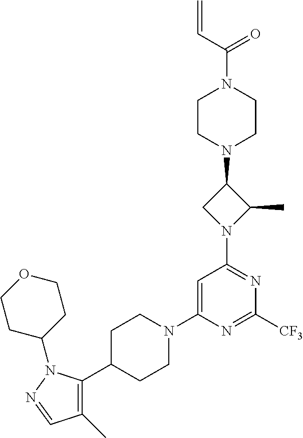 [SMILES: C=CC(=O)N1CCN([C@@H]2CN(c3cc(N4CCC(c5c(C)cnn5C5CCOCC5)CC4)nc(C(F)(F)F)n3)[C@@H]2C)CC1] | 1-(4-((2R,3R)-2- methyl-1-(6-(4-(4- methyl-1- (tetrahydro-2H- pyran-4-yl)-1H- pyrazol-5- yl)piperidin-1-yl)- 2-(trifluoromethyl) pyrimidin-4- yl)azetidin-3- yl)piperazin-1- yl)prop-2-en-1-one |
| 2-073 | 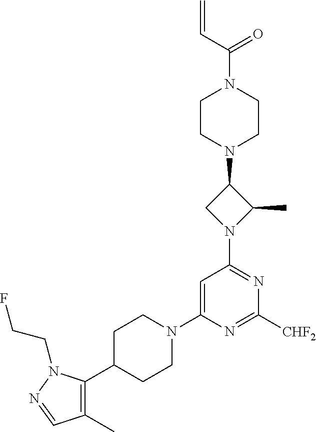 [SMILES: C=CC(=O)N1CCN([C@@H]2CN(c3cc(N4CCC(c5c(C)cnn5CCF)CC4)nc(C(F)F)n3)[C@@H]2C)CC1] | 1-(4-((2R,3R)-1-(2- (difluoromethyl)-6- (4-(1-(2- fluoroethyl)-4- methyl-1H- pyrazol-5- yl)piperidin-1- yl)pyrimidin-4-yl)- 2-methylazetidin- 3-yl)piperazin-1- yl)prop-2-en-1-one | 2-077 | 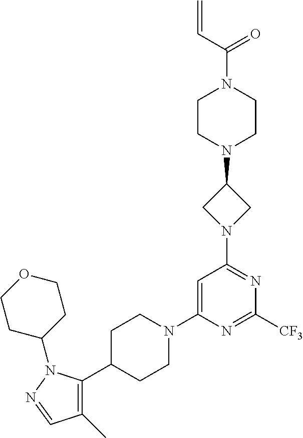 | methyl-1- (tetrahydro-2H- pyran-4-yl)-1H- pyrazol-5- yl)piperidin-1-yl)- 2-(trifluoromethyl) pyrimidint-4- yl)azetidin-3. yl)piperazin-1- yl)prop-2-en-1-one |
| 2-074 | 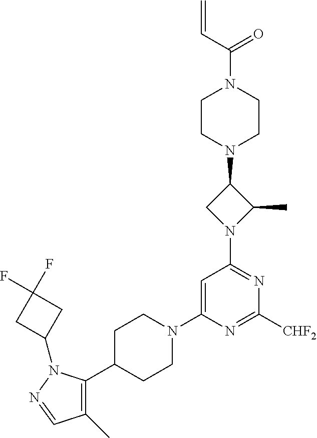 [SMILES: C=CC(=O)N1CCN([C@@H]2CN(c3cc(N4CCC(c5c(C)cnn5C5CC(F)(F)C5)CC4)nc(C(F)F)n3)[C@@H]2C)CC1] | 1-(4-((2R,3R)-1-(6- (4-(1-(3,3- difluorocyclobutyl)- 4-methyl-1H- pyrazol-5- yl)piperidin-1-yl)- 2-(difluoromethyl) pyrimidin-4-yl)-2- methylazetidin-3- yl)piperazin-1- yl)prop-2-en-1-one | 2-078 | 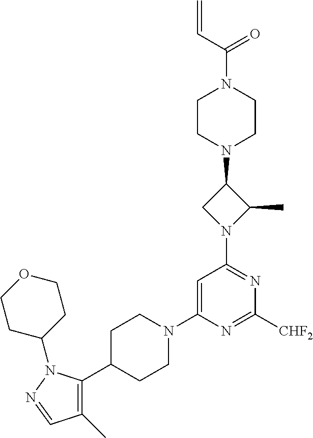 [SMILES: C=CC(=O)N1CCN([C@@H]2CN(c3cc(N4CCC(c5c(C)cnn5C5CCOCC5)CC4)nc(C(F)F)n3)[C@@H]2C)CC1] | 1-(4-(2R,3R)-1-(2- (difluoromethyl)-6- (4-(4-methyl-1- (tetrahydro-2H- pyran-4-yl)-1H- pyrazol-5- yl)piperidin-1- yl)pyrimidin-4-yl)- 2-methylazetidin- 3-yl)piperazin-1- yl)prop-2-en-1-one |
| 2-079 | 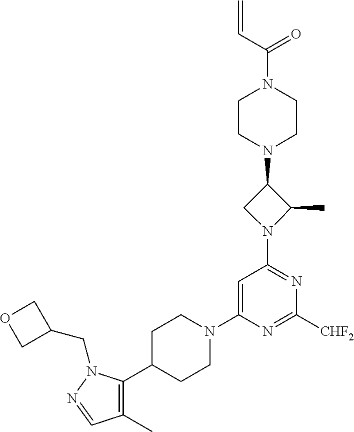 [SMILES: C=CC(=O)N1CCN([C@@H]2CN(c3cc(N4CCC(c5c(C)cnn5CC5COC5)CC4)nc(C(F)F)n3)[C@@H]2C)CC1] | 1-(4-((2R,3R)-1-(2- (difluoromethyl)-6- (4-(4-methyl-1- (oxetan-3- ylmethyl)-1H- pyrazol-5- yl)piperidin-1- yl)pyrimidin-4-yl)- 2-methylazetidin- 3-yl)piperazin-1- yl)prop-2-en-1-one | 2-083 | 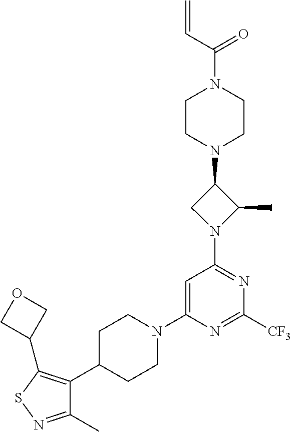 [SMILES: C=CC(=O)N1CCN([C@@H]2CN(c3cc(N4CCC(c5c(C)nsc5C5COC5)CC4)nc(C(F)(F)F)n3)[C@@H]2C)CC1] | 1-(4-((2R,3R)-2- methyl-1-(6-(4-(3- methyl-5-(oxetan- 3-yl)isothiazol-4- yl)piperidin-1-yl)- 2-(trifluoromethyl) pyrimidin-4- yl)azetidin-3- yl)piperazin-1- yl)prop-2-en-1-one |
| 2-080 | 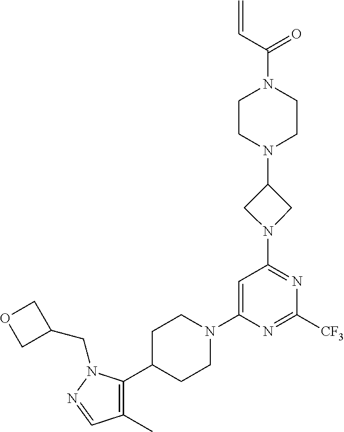 [SMILES: C=CC(=O)N1CCN(C2CN(c3cc(N4CCC(c5c(C)cnn5CC5COC5)CC4)nc(C(F)(F)F)n3)C2)CC1] | 1-(4-(1-(6-(4-(4- methyl-1-(oxetan- 3-ylmethyl)-1H- pyrazol-5- yl)piperidin-1-yl)- 2-(trifluoromethyl) pyrimidin-4- yl)azetidin-3- yl)piperazin-1- yl)prop-2-en-1-one | 2-084 | 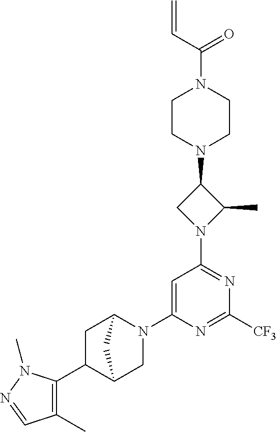 [SMILES: C=CC(=O)N1CCN([C@@H]2CN(c3cc(N4C[C@@H]5C[C@H]4CC5c4c(C)cnn4C)nc(C(F)(F)F)n3)[C@@H]2C)CC1] | 1-(4-((2R,3R)-1-(6- ((1S,4R)-5-(1,4- dimethyl-1H- pyrazol-5-yl)-2- azabicyclo[2.2.1] heptan-2-yl)-2- (trifluoromethyl) pyrimidin-4-yl)-2- methylazetidin-3- yl)piperazin-1- yl)prop-2-en-1-one |
| 2-081 | 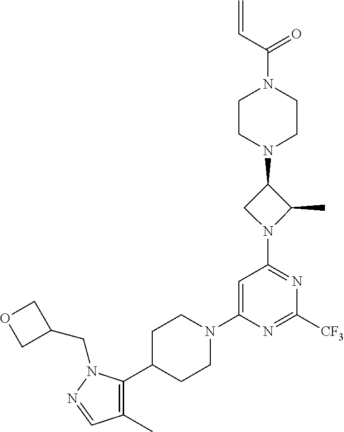 [SMILES: C=CC(=O)N1CCN([C@@H]2CN(c3cc(N4CCC(c5c(C)cnn5CC5COC5)CC4)nc(C(F)(F)F)n3)[C@@H]2C)CC1] | 1-(4-((2R.3R)-2- methyl-1-(6-(4-(4- methyl-1-(oxetan- 3-ylmethyl)-1H- pyrazol-5- yl)piperidin-1-yl)- 2-(trifluoromethyl) pyrimidin-4- yl)azetidin-3- yl)piperazin-1- yl)prop-2-en-1-one | 2-084-1 | 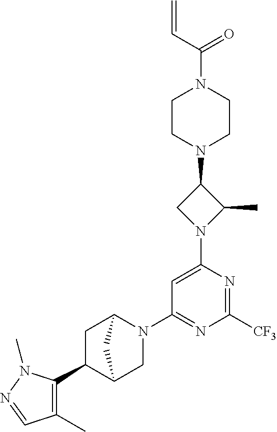 [SMILES: C=CC(=O)N1CCN([C@@H]2CN(c3cc(N4C[C@@H]5C[C@H]4C[C@@H]5c4c(C)cnn4C)nc(C(F)(F)F)n3)[C@@H]2C)CC1] | 1-(4-((2R,3R)-1-(6- ((1S,4R,5S)-5-(1,4- dimethyl-1H- pyrazol-5-yl)-2- azabicyclo[2.2.1] heptan-2-yl)-2- (trifluoromethyl) pyrimidin-4-yl)-2- methylazetidin-3- yl)piperazin-1- yl)prop-2-en-1-one |
| 2-082 | 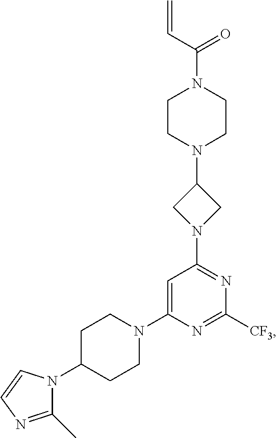 [SMILES: C=CC(=O)N1CCN(C2CN(c3cc(N4CCC(n5ccnc5C)CC4)nc(C(F)(F)F)n3)C2)CC1] | 1-(4-(1-(6-(4-(2- methyl-1H- imidazol-1- yl)piperidin-1-yl)- 2-(trifluoromethyl) pyrimidin-4- yl)azetidin-3- yl)piperazin-{- yl)prop-2-en-1-one | 2-084-2 | 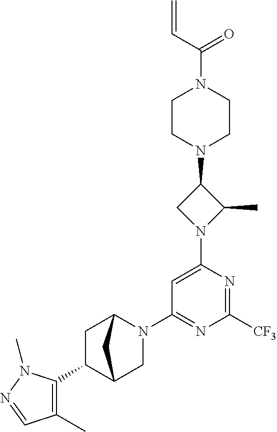 [SMILES: C=CC(=O)N1CCN([C@@H]2CN(c3cc(N4C[C@H]5C[C@@H]4C[C@H]5c4c(C)cnn4C)nc(C(F)(F)F)n3)[C@@H]2C)CC1] | 1-(4-((2R,3R)-1-(6- ((1R,4S,5R)-5-(1,4- dimethyl-1H- pyrazol-5-yl)-2- azabicyclo[2.2.]] heptan-2-yl)-2- (trifluoromethyl) pyrimidin-4-yl)-2- methylazetidin-3- yl)piperazin-1- yl)prop-2-en-1-one |
| 2-085 | 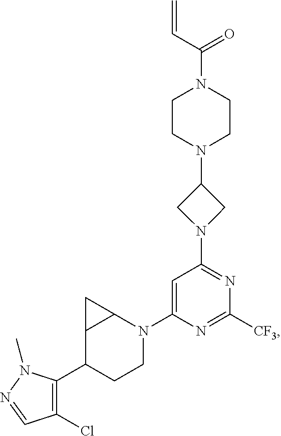 [SMILES: C=CC(=O)N1CCN(C2CN(c3cc(N4CCC(c5c(Cl)cnn5C)C5CC54)nc(C(F)(F)F)n3)C2)CC1] | 1-(4-(1-(6-(5-(4- chloro-1-methyl- 1H-pyrazol-5-yl)- 2-azabicyclo[4.1.0] heptan-2-yl)-2- (trifluoromethyl) pyrimidin-4- ylazetidin-3- yl)piperazin-1- yl)prop-2-en-1-one | 2-089 | 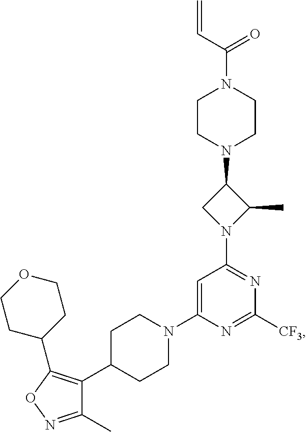 [SMILES: C=CC(=O)N1CCN([C@@H]2CN(c3cc(N4CCC(c5c(C)noc5C5CCOCC5)CC4)nc(C(F)(F)F)n3)[C@@H]2C)CC1] | 1-(4-((2R,3R)-2- methyl-1-(6-(4-(3- methyl-5- (tetrahydro-2H- pyran-4- yl)isoxazol-4- yl)piperidin-1-yl)- 2-(trifluoromethyl) pyrimidin-4- ylazetidin-3- yl)piperazin-1- yl)prop-2-en-1-one |
| 2-086 | 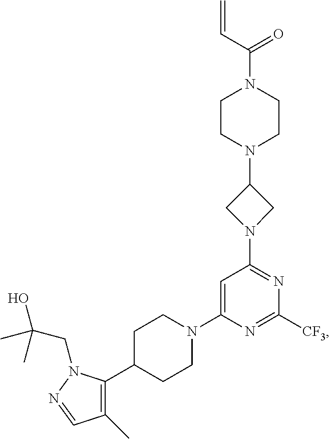 [SMILES: C=CC(=O)N1CCN(C2CN(c3cc(N4CCC(c5c(C)cnn5CC(C)(C)O)CC4)nc(C(F)(F)F)n3)C2)CC1] | 1-(4-(1-(6-(4-(1-(2- hydroxy-2- methylpropyl)-4- methyl-1H- pyrazol-5- yl)piperidin-1-yl)- 2-(trifluoromethyl) pyrimidin-4- yl)azetidin-3- yl)piperazin-1- yl)prop-2-en-1-one | 2-090 | 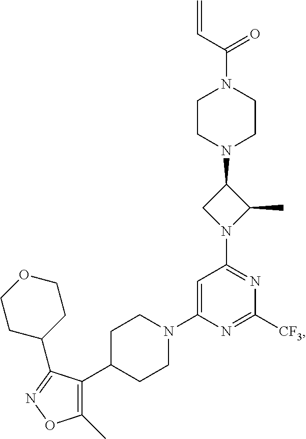 [SMILES: C=CC(=O)N1CCN([C@@H]2CN(c3cc(N4CCC(c5c(C6CCOCC6)noc5C)CC4)nc(C(F)(F)F)n3)[C@@H]2C)CC1] | 1-(4-((2R,3R)-2- methyl-1-(6-(4-(5- methyl-3- (tetrahydro-2H- pyran-4- yl)isoxazol-4- yl)piperidin-1-yl)- 2-(trifluoromethyl) pyrimidin-4- ylazetidin-3- yl)piperazin-1- yl)prop-2-en-1-one |
| 2-087 | 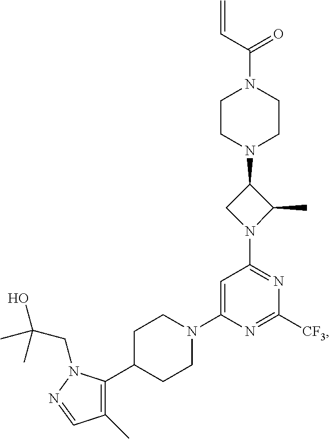 [SMILES: C=CC(=O)N1CCN([C@@H]2CN(c3cc(N4CCC(c5c(C)cnn5CC(C)(C)O)CC4)nc(C(F)(F)F)n3)[C@@H]2C)CC1] | 1-(4-((2R,3R)-1-(6- (4-(1-(2-hydroxy- 2-methylpropyl)-4- methyl-1H- pyrazol-5- yl)piperidin-1-yl)- 2-(trifluoromethyl) pyrimidin-4-yl)-2- methylazetidin-3. yl)piperazin-1- yl)prop-2-en-1-one | 2-091 | 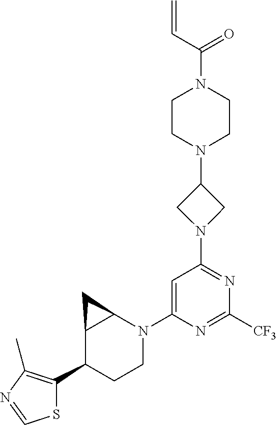 [SMILES: C=CC(=O)N1CCN(C2CN(c3cc(N4CC[C@@H](c5scnc5C)[C@H]5C[C@H]54)nc(C(F)(F)F)n3)C2)CC1] | 1-(4-(1-(6- ((1R,5R,6R)-5-(4- methylthiazol-5- yl)-2- azabicyclo[4.1.0] heptan-2-yl)-2- (trifluoromethyl) pyrimidin-4- yl)azetidin-3- yl)piperaziu-1- yl)prop-2-en-1-one |
| 2-088 | 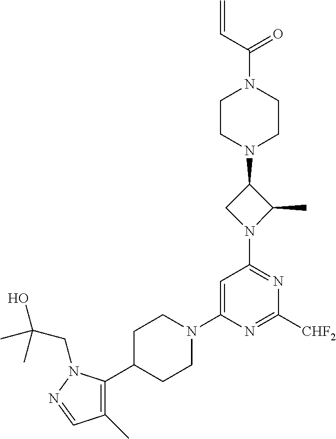 [SMILES: C=CC(=O)N1CCN([C@@H]2CN(c3cc(N4CCC(c5c(C)cnn5CC(C)(C)O)CC4)nc(C(F)F)n3)[C@@H]2C)CC1] | 1-(4-((2,3R)-1-(2- (difluoromethyl)-6- (4-(1-(2-hydroxy- 2-methylpropyl)-4- methyl-1H- pyrazol-5- yl)piperidin-1- yl)pyrimidin-4-y])- 2-methylazetidin- 3-yl)piperazin-1- yl)prop-2-en-1-one | 2-092 | 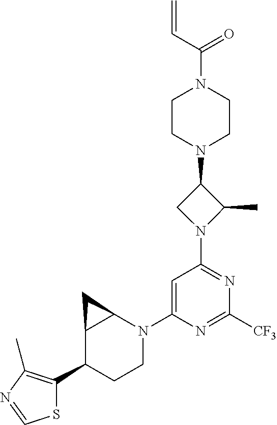 [SMILES: C=CC(=O)N1CCN([C@@H]2CN(c3cc(N4CC[C@@H](c5scnc5C)[C@H]5C[C@H]54)nc(C(F)(F)F)n3)[C@@H]2C)CC1] | 1-(4-((2R,3R)-2- methyl-1-(6- ((1R,SR,6R)-5-(4- methylthiazol-5- yl)-2- azabicyclo[4.1.0] heptan-2-yl)-2- (trifluoromethyl) pyrimidin-4- yl)azetidin-3- yl)piperazin-1- yl)prop-2-en-1-one |
| 2-093 | 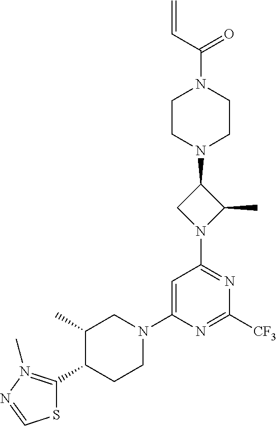 | 1-(4-((2R,3R)-2- methyl-1-(6- ((3S,4S)-3-methyl- 4-(4-methylthiazol- 5-yl)piperidin-1- yl)-2- (trifluoromethyl) pyrimidin-4- yl)azetidin-3- yl)piperazin-1- yl)prop-2-en-1-one | 2-097 | 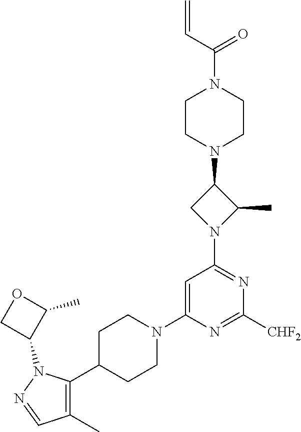 [SMILES: C=CC(=O)N1CCN([C@@H]2CN(c3cc(N4CCC(c5c(C)cnn5[C@@H]5CO[C@@H]5C)CC4)nc(C(F)F)n3)[C@@H]2C)CC1] | 1-(4-((2R,3R)-1-(2- (difluoromethyl)-6- (4-(4-methyl-1- ((2R,3R)-2- methyloxetan-3- yl)-1H-pyrazol-5- yl)piperidin-1- yl)pyrimidin-4-yl)- 2-methylazetidin- 3-yl)piperazin-1- yl)prop-2-en-1-one |
| 2-094 | 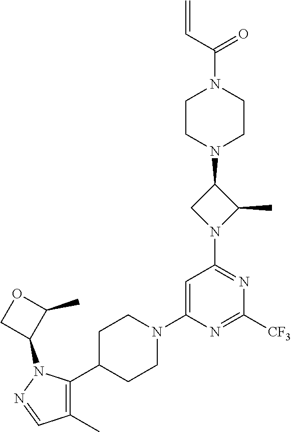 [SMILES: C=CC(=O)N1CCN([C@@H]2CN(c3cc(N4CCC(c5c(C)cnn5[C@H]5CO[C@H]5C)CC4)nc(C(F)(F)F)n3)[C@@H]2C)CC1] | 1-(4-((2R,3R)-2- methyl-1-(6-(4-(4- methyl-1-((2S,3S)- 2-methyloxetan-3- yl)-1H-pyrazol-5- yl)piperidin-1-yl)- 2-(trifluoromethyl) pyrimidin-4- yl)azetidin-3- yl)piperazin-1- yl)prop-2-en-1-one | 2-098 | 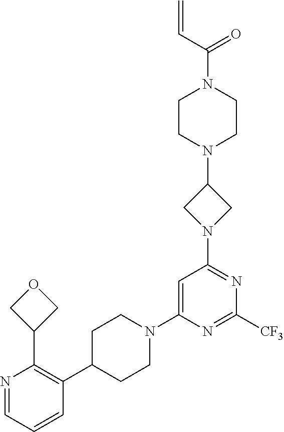 [SMILES: C=CC(=O)N1CCN(C2CN(c3cc(N4CCC(c5cccnc5C5COC5)CC4)nc(C(F)(F)F)n3)C2)CC1] | 1-(4-(1-(6-(4-(2- (oxetan-3- ylpyridin-3- yl)piperidin-1-yl)- 2-(trifluoromethyl) pyrimidin-4- yl)azetidin-3- yl)piperazin-1- yl)prop-2-en-1-one |
| 2-095 | 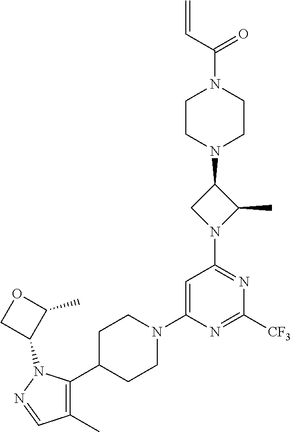 [SMILES: C=CC(=O)N1CCN([C@@H]2CN(c3cc(N4CCC(c5c(C)cnn5[C@@H]5CO[C@@H]5C)CC4)nc(C(F)(F)F)n3)[C@@H]2C)CC1] | 1-(4-((2R.3R)-2- methyl-1-(6-(4-(4- methyl-1-((2R,3R)- 2-methyloxetan-3- yl)-1H-pyrazol-5- yl)piperidin-1-yl)- 2-(trifluoromethyl) pyrimidin-4- yl)azetidin-3- yl)piperazin-1- yl)prop-2-en-1-one | 2-099 | 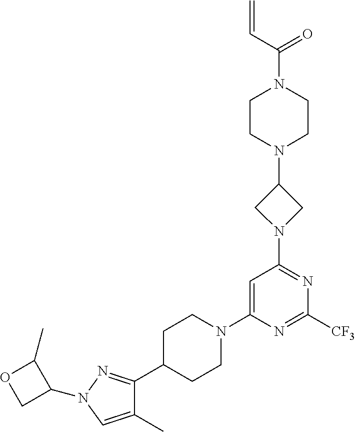 [SMILES: C=CC(=O)N1CCN(C2CN(c3cc(N4CCC(c5nn(C6COC6C)cc5C)CC4)nc(C(F)(F)F)n3)C2)CC1] | 1-(4-(1-(6-(4-(4- methyl-1-(2- methyloxetan-3- yl)-1H-pyrazol-3- yl)piperidin-1-yl)- 2-(trifluoromethyl) pyrimidin-4- ylazetidin-3- yl)piperazin-1- yl)prop-2-en-1-one |
| 2-096 | 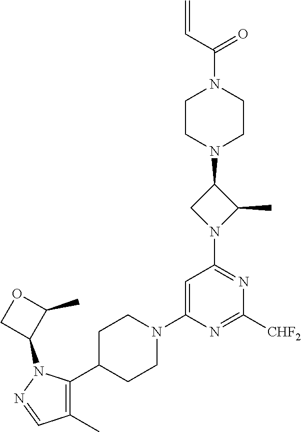 [SMILES: C=CC(=O)N1CCN([C@@H]2CN(c3cc(N4CCC(c5c(C)cnn5[C@H]5CO[C@H]5C)CC4)nc(C(F)F)n3)[C@@H]2C)CC1] | 1-(4-((2R,3R)-1-(2- (difluoromethyl)-6- (4-(4-methyl-1- ((2S,3S)-2- methyloxetan-3- yl)-1H-pyrazol-5- yl)piperidin-1- yl)pyrimidin-4-y])- 2-methylazetidin- 3-yl)piperazin-1- yl)prop-2-en-1-one | 2-100 | 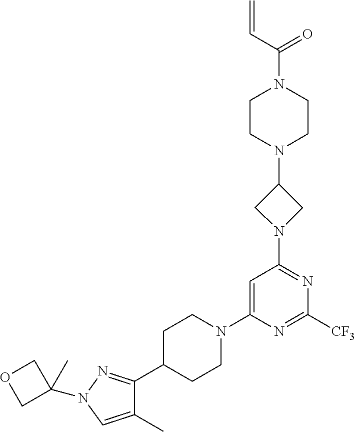 [SMILES: C=CC(=O)N1CCN(C2CN(c3cc(N4CCC(c5nn(C6(C)COC6)cc5C)CC4)nc(C(F)(F)F)n3)C2)CC1] | 1-(4-(1-(6-(4-(4- methyl-1-(3- methyloxetan-3- yl)-1H-pyrazol-3- yl)piperidin-1-yl)- 2-(trifluoromethyl) pyrimidin-4- yl)azetidin-3- ylpiperazin-1- yl)prop-2-en-1-one |
| 2-101 | 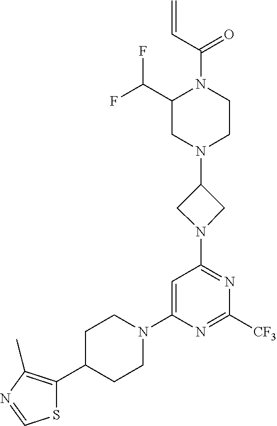 [SMILES: C=CC(=O)N1CCN(C2CN(c3cc(N4CCC(c5scnc5C)CC4)nc(C(F)(F)F)n3)C2)CC1C(F)F] | 1-(2- (difluoromethyl)-4- (1-(6-(4-(4- methylthiazol-5- yl)piperidin-1-yl)- 2-(trifluoromethyl) pyrimidin-4- yl)azetidin-3- yl)piperazin-1- yl)prop-2-en-1-one | 2-103 | 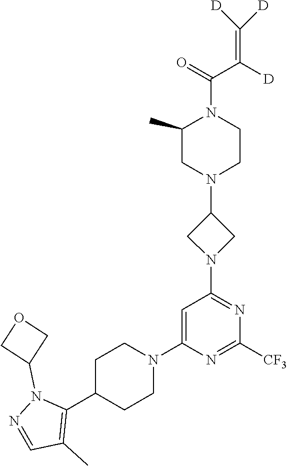 [SMILES: [2H]C([2H])=C([2H])C(=O)N1CCN(C2CN(c3cc(N4CCC(c5c(C)cnn5C5COC5)CC4)nc(C(F)(F)F)n3)C2)C[C@H]1C] | (R)-1-(2-methyl-4- (1-(6-(4-(4-methyl- 1-(oxetan-3-yl)- 1H-pyrazol-5- yl)piperidin-1-yl)- 2.(trifluoromethyl) pyrimidin-4- yl)azetidin-3- yl)piperazin-1- yl)prop-2-en-1- one-2,3,3-d3 |
| 2-101-1 | 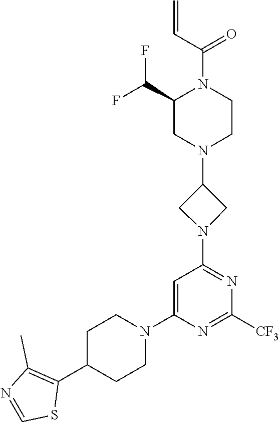 [SMILES: C=CC(=O)N1CCN(C2CN(c3cc(N4CCC(c5scnc5C)CC4)nc(C(F)(F)F)n3)C2)C[C@H]1C(F)F] | (S)-1-(2- (difluoromethyl)-4- (1-(6-(4-(4- methylthiazol-5- yl)piperidin-1-yl)- 2-(trifluoromethyl) pyrimidin-4- yl)azetidin-3- yl)piperazin-1- yl)prop-2-en-1-one | 2-104 | 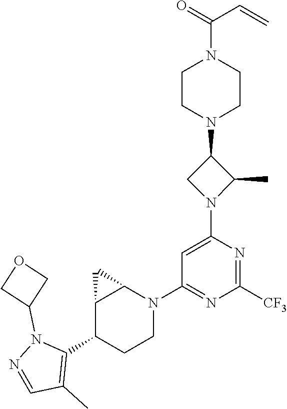 [SMILES: C=CC(=O)N1CCN([C@@H]2CN(c3cc(N4CC[C@H](c5c(C)cnn5C5COC5)[C@@H]5C[C@@H]54)nc(C(F)(F)F)n3)[C@@H]2C)CC1] | 1-(4-((2R,3R)-2- methyl-1-(6- ((1S,5S,6S)-5-(4- methyl-1-(oxetan- 3-yl)-1H-pyrazol- 5-yl)-2- azabicyclo[4.1.0] heptan-2-yl)-2- (trifluoromethyl) pyrimidin-4- yl)azetidin-3- yl)piperazin-1- yl)prop-2-en-1-one |
| 2-101-2 | 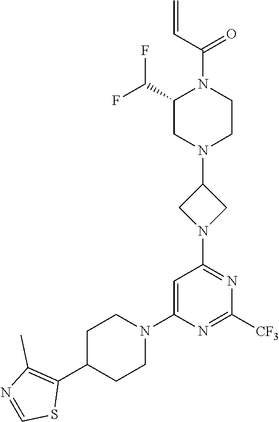 [SMILES: C=CC(=O)N1CCN(C2CN(c3cc(N4CCC(c5scnc5C)CC4)nc(C(F)(F)F)n3)C2)C[C@@H]1C(F)F] | (R)-1-(2- (difluoromethyl)-4- (1-(6-(4-(4- methylthiazol-5- yl)piperidin-1-yl)- 2- (trifluoromethyl) pyrimidin-4- yl)azetidin-3- yl)piperazin-1- yl)prop-2-en-1-one | 2-105 | 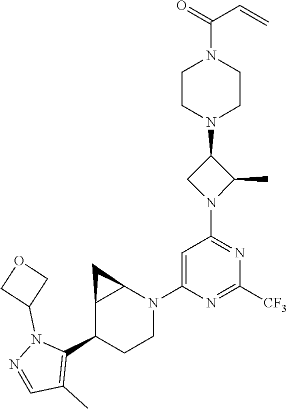 [SMILES: C=CC(=O)N1CCN([C@@H]2CN(c3cc(N4CC[C@@H](c5c(C)cnn5C5COC5)[C@H]5C[C@H]54)nc(C(F)(F)F)n3)[C@@H]2C)CC1] | 1-(4-((2R,3R)-2- methyl-1-(6- ((IR,SR,6R)-5-(4- methyl-1-(oxetan- 3-yl)-1H-pyrazol- 5-yl)-2- azabicyclo[4.1.0] heptan-2-yl)-2- (trifluoromethyl) pyrimidin-4- yl)azetidin-3- yl)piperazin-1- yl)prop-2-en-1-one |
| 2-102 | 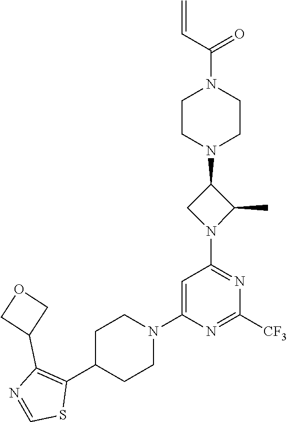 [SMILES: C=CC(=O)N1CCN([C@@H]2CN(c3cc(N4CCC(c5scnc5C5COC5)CC4)nc(C(F)(F)F)n3)[C@@H]2C)CC1] | 1-(4-((2R,3R)-2- methyl-1-(6-(4-(4- (oxetan-3- yl)thiazol-5. yl)piperidin-1-yl)- 2-(trifluoromethyl) pyrimidin-1- yl)azetidin-3- yl)piperazin-1- yl)prop-2-en-1-one | 2-106 | 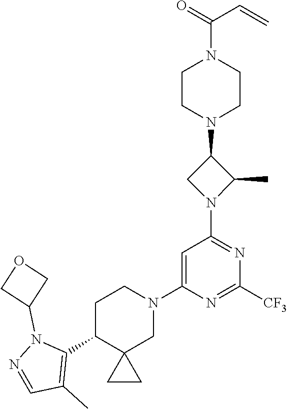 [SMILES: C=CC(=O)N1CCN([C@@H]2CN(c3cc(N4CC[C@@H](c5c(C)cnn5C5COC5)C5(CC5)C4)nc(C(F)(F)F)n3)[C@@H]2C)CC1] | 1-(4-((2R,3R)-2- methy-1-1-(6-((R)-8- (4-methyl-1- (oxetan-3-yl)-1H- pyrazol-5-yl)-5- azaspiro[2.5]octan- 5-yl)-2- (trifluoromethyl) pyrimidin-4- yl)azetidin-3- yl)piperazin-1- yl)prop-2-en-1-one |
| 2-107 | 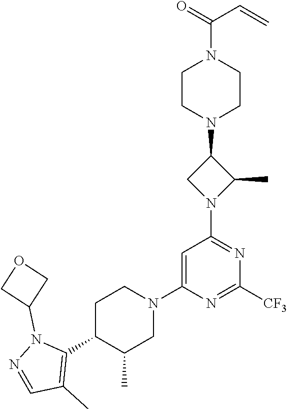 [SMILES: C=CC(=O)N1CCN([C@@H]2CN(c3cc(N4CC[C@@H](c5c(C)cnn5C5COC5)[C@@H](C)C4)nc(C(F)(F)F)n3)[C@@H]2C)CC1] | 1-(4-((2R,3R)-2- methyl-1-16- ((3R,4R)-3-methyl- 4-(4-methyl-1- (oxetan-3-yl)-1H- pyrazol-5- yl)piperidin-1-yl)- 2-(trifluoromethyl) pyrimidin-4- ylazetidin-3- yl)piperazin-1- yl)prop-2-en-1-one | 2-111 | 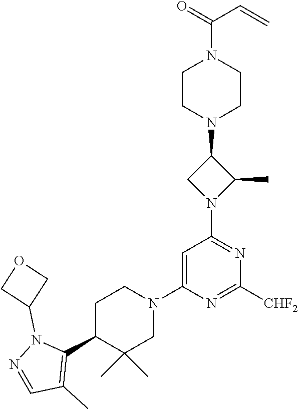 [SMILES: C=CC(=O)N1CCN([C@@H]2CN(c3cc(N4CC[C@H](c5c(C)cnn5C5COC5)C(C)(C)C4)nc(C(F)F)n3)[C@@H]2C)CC1] | 1-(4-((2R.3R)-1-(2- (difluoromethyl)-6- ((S)-3,3-dimethyl- 4-(4-methyl-1- (oxetan-3-yl)-1H- pyrazol-5- yl)piperidin-1- yl)pyrimidin-4-yl)- 2-methylazetidin- 3-yl)piperazin-1- yl)prop-2-en-1-one |
| 2-108 | 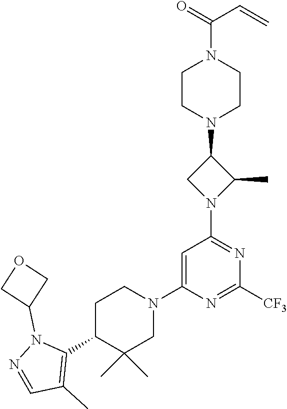 [SMILES: C=CC(=O)N1CCN([C@@H]2CN(c3cc(N4CC[C@@H](c5c(C)cnn5C5COC5)C(C)(C)C4)nc(C(F)(F)F)n3)[C@@H]2C)CC1] | 1-(4-((2R,3R)-1-(6- ((R)-3,3-dimethyl- 4-(4-methyl-1- (oxetan-3-yl)-1H- pyrazol-5- yl)piperidin-1-yl)- 2-(trifluoromethyl) pyrimidin-4-yl)-2- methylazetidin-3- yl)piperazin-1- yl)prop-2-en-1-one | 2-112 | 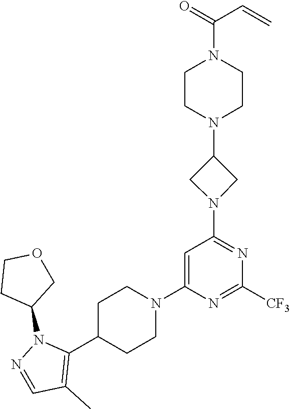 [SMILES: C=CC(=O)N1CCN(C2CN(c3cc(N4CCC(c5c(C)cnn5[C@H]5CCOC5)CC4)nc(C(F)(F)F)n3)C2)CC1] | (S)-1-(4-(1-(6-(4- (4-methyl-1- (tetrahydrofuran-3- yl)-1H-pyrazol-5- yl)piperidin-1-yl)- 2-(trifluoromethyl) pyrimidin-4- yl)azetidin-3- yl)piperazin-1- yl)prop-2-en-1-one |
| 2-109 | 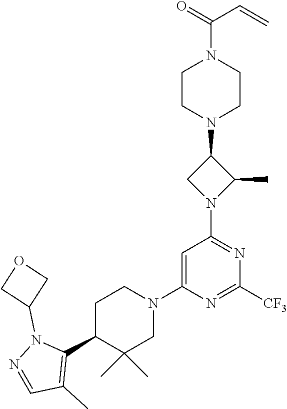 [SMILES: C=CC(=O)N1CCN([C@@H]2CN(c3cc(N4CC[C@H](c5c(C)cnn5C5COC5)C(C)(C)C4)nc(C(F)(F)F)n3)[C@@H]2C)CC1] | 1-(4-((2R,3R)-1-(6- ((S)-3,3-dimethyl- 4-(4-methyl-1- (oxetan-3-yl)-1H- pyrazol-5- yl)piperidin-1-yl)- 2-(trifluoromethyl) pyrimidin-4-yl)-2- methylazetidin-3- yl)piperazin-1- yl)prop-2-en-1-one | 2-113 | 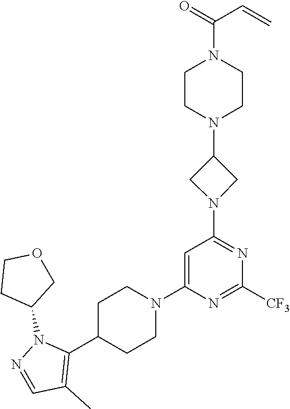 [SMILES: C=CC(=O)N1CCN(C2CN(c3cc(N4CCC(c5c(C)cnn5[C@@H]5CCOC5)CC4)nc(C(F)(F)F)n3)C2)CC1] | ((R)-1-(4-(1-(6-(4- (4-methyl-1- (tetrahydrofuran-3- yl)-1H-pyrazol-5- yl)piperidin-1-yl)- 2-(trifluoromethyl) pyrimidin-4- ylazetidin-3- yl)piperazin-1- yl)prop-2-en-1-one |
| 2-110 | 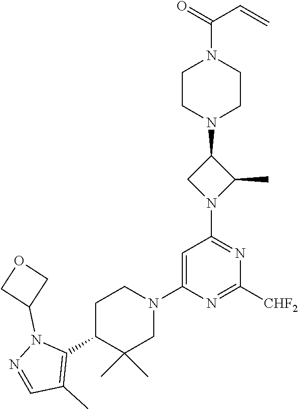 [SMILES: C=CC(=O)N1CCN([C@@H]2CN(c3cc(N4CC[C@@H](c5c(C)cnn5C5COC5)C(C)(C)C4)nc(C(F)F)n3)[C@@H]2C)CC1] | 1-(4-((2R,3R)-1-(2- (difluoromethyl)-6- ((R)-3.3-dimethyl- 4-(4-methyl-1- (oxctan-3-yl)-1H- pyrazol-5- yl)piperidin-1- yl)pyrimidin-4-y])- 2-methylazetidin- 3-yl)piperazin-1- yl)prop-2-en-1-one | 2-114 | 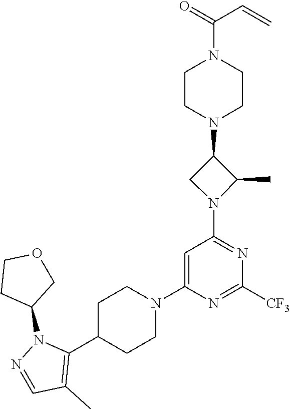 [SMILES: C=CC(=O)N1CCN([C@@H]2CN(c3cc(N4CCC(c5c(C)cnn5[C@H]5CCOC5)CC4)nc(C(F)(F)F)n3)[C@@H]2C)CC1] | 1-(4-((2R,3R)-2- methyl-1-(6-(4-(4- methyl-1-((S)- tetrahydrofuran-3- yl)-1H-pyrazol-5- yl)piperidin-1-yl)- 2-(trifluoromethyl) pyrimidin-4- yl)azetidin-3- yl)ypiperazin-1- yl)prop-2-en-1-one |
| 2-115 | 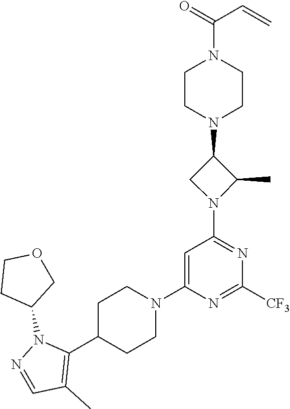 [SMILES: C=CC(=O)N1CCN([C@@H]2CN(c3cc(N4CCC(c5c(C)cnn5[C@@H]5CCOC5)CC4)nc(C(F)(F)F)n3)[C@@H]2C)CC1] | 1-(4-((2R,3R)-2- methyl-1-(6-(4-(4- methyl-1-((R)- tetrahydrofuran-3- yl)-1H-pyrazol-5- yl)piperidin-[1-yl)- 2-(trifluoromethyl) pyrimidin-4- yl)azetidin-3- yl)piperazin-1- yl)prop-2-en-1-one | 2-119 | 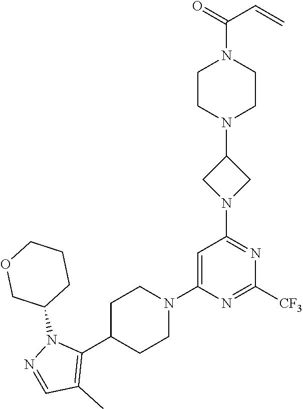 [SMILES: C=CC(=O)N1CCN(C2CN(c3cc(N4CCC(c5c(C)cnn5[C@H]5CCCOC5)CC4)nc(C(F)(F)F)n3)C2)CC1] | (S)-1-(4-(1-(6-(4- (4-methyl-1- (tetrahydro-2H- pyran-3-yl)-1H- pyrazol-5- yl)piperidin-1-yl)- 2-(trifluoromethyl) pyrimidin-4- yl)azetidin-3- yl)piperazin-1- yl)prop-2-en-1-one |
| 2-116 | 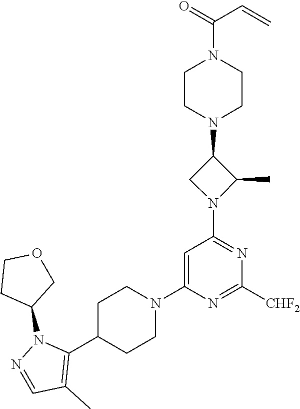 [SMILES: C=CC(=O)N1CCN([C@@H]2CN(c3cc(N4CCC(c5c(C)cnn5[C@H]5CCOC5)CC4)nc(C(F)F)n3)[C@@H]2C)CC1] | 1-(4-((2R,3R)-1-(2- (difluoromethyl)-6- (4-(4-methyl-1- ((S)- tetrahydrofuran-3- yl)-1H-pyrazol-5- yl)piperidin-1- yl)pyrimidin-4-yl)- 2-methylazetidin- 3-yl)piperazin-1- yl)prop-2-en-1-one | 2-120 | 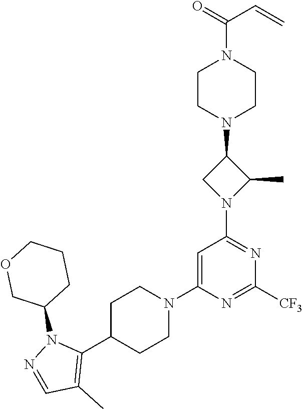 [SMILES: C=CC(=O)N1CCN([C@@H]2CN(c3cc(N4CCC(c5c(C)cnn5[C@@H]5CCCOC5)CC4)nc(C(F)(F)F)n3)[C@@H]2C)CC1] | 1-(4-((2R,3R)-2- methyl-1-(6-(4-(4- methyl-1-((R)- tetrahydro-2H- pyran-3-yl)-1H- pyrazol-5- yl)piperidin-1-yl)- 2-(trifluoromethyl) pyrimidin-4- ylazetidin-3- yl)piperazin-1- yl)prop-2-en-1-one |
| 2-117 | 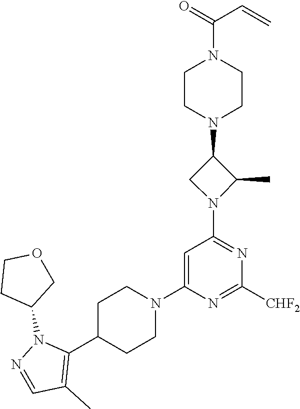 [SMILES: C=CC(=O)N1CCN([C@@H]2CN(c3cc(N4CCC(c5c(C)cnn5[C@@H]5CCOC5)CC4)nc(C(F)F)n3)[C@@H]2C)CC1] | 1-(4-((2R,3R)-1-(2- (difluoromethyl)-6- (4-(4-methyl-1- ((R)- tetrabydrofuran-3- yl)-1H-pyrazol-5- yl)piperidin-1- yl)pyrimidin-4-yl)- 2-methylazetidin- 3-yl)piperazin-1- yl)prop-2-en-1-one | 2-121 | 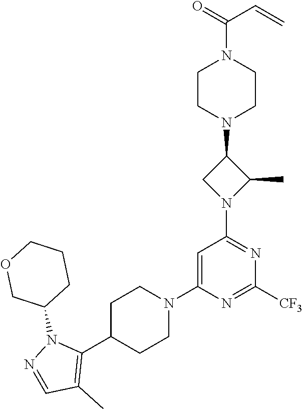 [SMILES: C=CC(=O)N1CCN([C@@H]2CN(c3cc(N4CCC(c5c(C)cnn5[C@H]5CCCOC5)CC4)nc(C(F)(F)F)n3)[C@@H]2C)CC1] | 1-(4-((2R,3R)-2- methyl-1-(6-(4-(4- methyl-1-((S)- tetrahydro-2H- pyran-3-yl)-1H- pyrazol-5- yl)piperidin-1-yl)- 2-(trifluoromethyl) pyrimidin-4- yl)azetidin-3- yl)piperazin-1- yl)prop-2-en-1-one |
| 2-118 | 1834 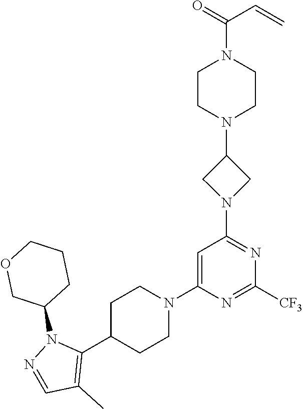 [SMILES: C=CC(=O)N1CCN(C2CN(c3cc(N4CCC(c5c(C)cnn5[C@@H]5CCCOC5)CC4)nc(C(F)(F)F)n3)C2)CC1] | (R)-1-(4-(1-(6-(4- (4-methyl-1- (tetrahydro-2H- pyran-3-yl)-1H- pyrazol-5- yl)piperidin-1-yl)- 2-(trifluoromethyl) pyrimidin-4- ylazetidin-3- yl)piperazin-1- yl)prop-2-en-1-one | 2-122 | 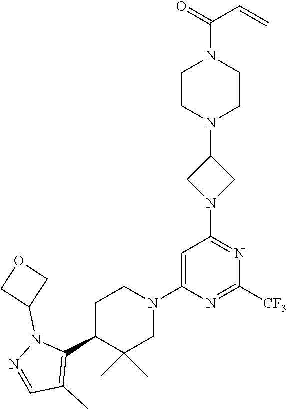 [SMILES: C=CC(=O)N1CCN(C2CN(c3cc(N4CC[C@H](c5c(C)cnn5C5COC5)C(C)(C)C4)nc(C(F)(F)F)n3)C2)CC1] | (S)-1-(4-(1-(6-(3,3- dimethy-1-4-(4- methyl-1-(oxetan- 3-yl)-1H-pyrazo]- 5-yl)piperidin-1- yl)-2- (trifluoromethyl) pyrimidin-4- yl)azetidin-3- yl)piperazin-1- yl)prop-2-en-1-one |
| 2-123 | 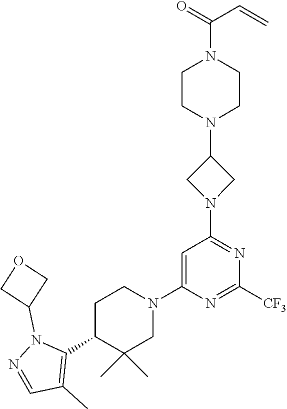 [SMILES: C=CC(=O)N1CCN(C2CN(c3cc(N4CC[C@@H](c5c(C)cnn5C5COC5)C(C)(C)C4)nc(C(F)(F)F)n3)C2)CC1] | (R)-1-(4-(1-(6-(3,3- dimethyl-4-(4- methyl-1-(oxetan- 3-yl)-1H-pyrazol- 5-yl)piperidin-1- yl)-2- (trifluoromethyl) pyrimidin-4- yl)azetidin-3- yl)piperazin-1- yl)prop-2-en-1-one | 2-127 | 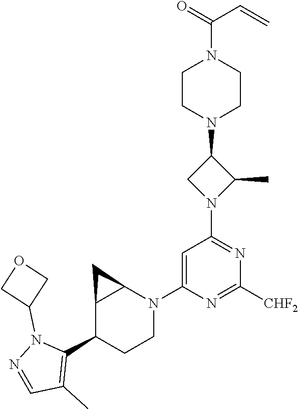 [SMILES: C=CC(=O)N1CCN([C@@H]2CN(c3cc(N4CC[C@@H](c5c(C)cnn5C5COC5)[C@H]5C[C@H]54)nc(C(F)F)n3)[C@@H]2C)CC1] | 1-(4-((2R,3R)-1-(2- (difluoromethyl)-6- ((1R,SR,6R)-5-(4- methyl-1-(oxetan- 3-yl)-1H-pyrazol- 5-yl)-2- azabicyclo[4.1.0] heptan-2- yl)pyrimidin-4-yl)- 2-methylazetidin- 3-yl)piperazin-1- yl)prop-2-en-1-one |
| 2-124 | 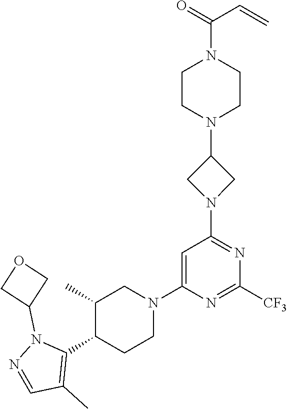 [SMILES: C=CC(=O)N1CCN(C2CN(c3cc(N4CC[C@H](c5c(C)cnn5C5COC5)[C@H](C)C4)nc(C(F)(F)F)n3)C2)CC1] | 1-(4-(1-(6-((3S,4S)- 3-methyl-4-(4- methyl-1-(oxetan- 3-yl)-1H-pyrazol- 5-yl)piperidin-1- yl)-2- (trifluoromethyl) pyrimidin-4- yl)azetidin-3- yl)piperazin-1- yl)prop-2-en-1-one | 2-128 | 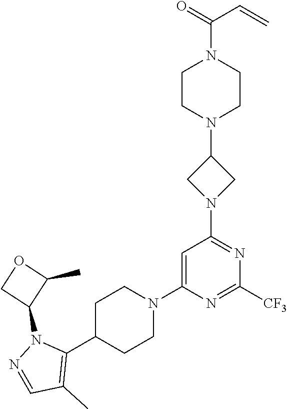 [SMILES: C=CC(=O)N1CCN(C2CN(c3cc(N4CCC(c5c(C)cnn5[C@H]5CO[C@H]5C)CC4)nc(C(F)(F)F)n3)C2)CC1] | 1-(4-(1-(6-(4-(4- methyl-1-((2S,3S)- 2-methyloxetan-3- yl)-1H-pyrazol-5- yl)piperidin-1-yl)- 2-(trifluoromethyl) pyrimidin-4- yl)azetidin-3- yl)piperazin-1- yl)prop-2-en-1-one |
| 2-125 | 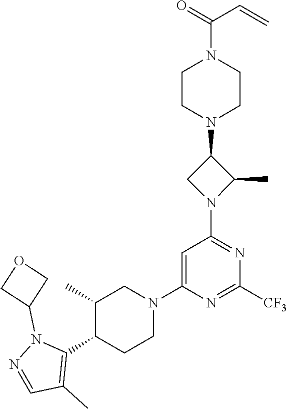 [SMILES: C=CC(=O)N1CCN([C@@H]2CN(c3cc(N4CC[C@H](c5c(C)cnn5C5COC5)[C@H](C)C4)nc(C(F)(F)F)n3)[C@@H]2C)CC1] | 1-(4-((2R,3R)-2- methyl-1-(6- ((3S,4S)-3-methyl- 4-(4-methyl-1- (oxetan-3-yl)-1H- pyrazol-5- yl)piperidin-[1-yl)- 2-(trifluoromethyl) pyrimidin-4- yl)azetidin-3- yl)piperazin-1- yl)prop-2-en-1-one | 2-129 | 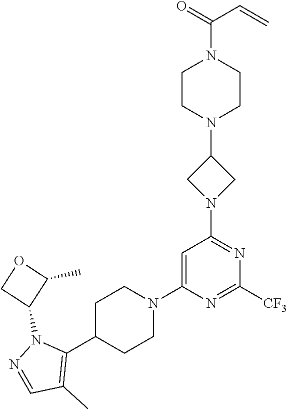 [SMILES: C=CC(=O)N1CCN(C2CN(c3cc(N4CCC(c5c(C)cnn5[C@@H]5CO[C@@H]5C)CC4)nc(C(F)(F)F)n3)C2)CC1] | 1-(4-(1-(6-(4-(4- methyl-1-((2,3R)- 2-methyloxetan-3- yl)-1H-pyrazol-5- yl)piperidin-1-yl)- 2-(trifluoromethyl) pyrimidin-4- yl)azetidin-3- yl)piperaziu-1- yl)prop-2-en-1-one |
| 2-126 | 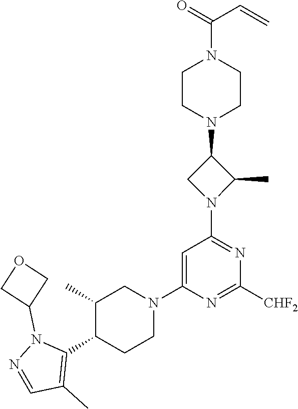 [SMILES: C=CC(=O)N1CCN([C@@H]2CN(c3cc(N4CC[C@H](c5c(C)cnn5C5COC5)[C@H](C)C4)nc(C(F)F)n3)[C@@H]2C)CC1] | 1-(4-((2R,3R)-1-(2- (difluoromethyl)-6- ((3S,4S)-3-methyl- 4-(4-methyl-1- (oxctan-3-yl)-1H- pyrazol-5- yl)piperidin-1- yl)pyrimidin-4-yl)- 2-methylazetidin- 3-yl)piperazin-1- yl)prop-2-en-1-one | 2-130 | 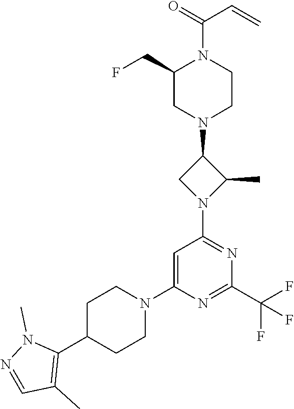 [SMILES: C=CC(=O)N1CCN([C@@H]2CN(c3cc(N4CCC(c5c(C)cnn5C)CC4)nc(C(F)(F)F)n3)[C@@H]2C)C[C@H]1CF] | 1-((S)-4-((2R,3R)- 1-(6-(4-(1,4- dimethyl-1H- pyrazol-5- yl)piperidin-1-yl)- 2-(trifluoromethyl) pyrimidin-4-yl)-2- methylazetidin-3- yl)-2- (fluoromethyl) piperazin-1- yl)prop-2- en-1-one |
| 2-131 | 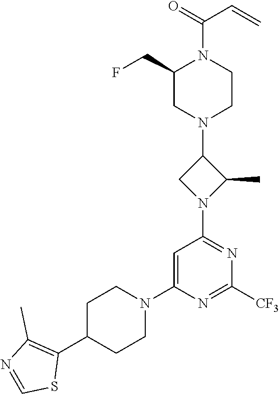 [SMILES: C=CC(=O)N1CCN(C2CN(c3cc(N4CCC(c5scnc5C)CC4)nc(C(F)(F)F)n3)[C@@H]2C)C[C@H]1CF] | 1-((2S)-2- (fluoromethyl)-4- ((2R)-2-methyl-1- (6-(4-(4- methylthiazol-5- yl)piperidin-1-yl)- 2-(trifluoromethyl) pyrimidin-4- yl)azetidin-3- yl)piperazin-1- yl)prop-2-en-1-one | 2-135 | 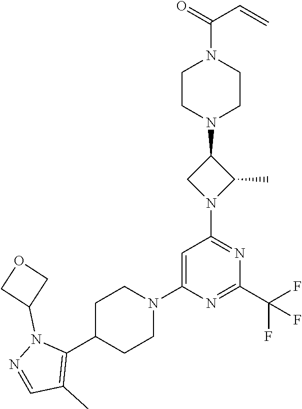 [SMILES: C=CC(=O)N1CCN([C@@H]2CN(c3cc(N4CCC(c5c(C)cnn5C5COC5)CC4)nc(C(F)(F)F)n3)[C@H]2C)CC1] | 1-(4-((28,3R)-2- methy-1-1-(6-(4-(4- methyl-1-(oxetan- 3-yl)-1H-pyrazol- 5-yl)piperidin-1- yl)-2- (trifluoromethyl) pyrimidin-4- yl)azetidin-3- yl)piperazin-1- yl)prop-2-en-1-one |
| 2-132 | 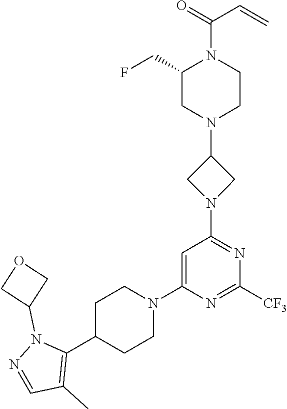 [SMILES: C=CC(=O)N1CCN(C2CN(c3cc(N4CCC(c5c(C)cnn5C5COC5)CC4)nc(C(F)(F)F)n3)C2)C[C@@H]1CF] | (R)-1-(2- (fluoromethyl)-4- (1-(6-(4-(4-methyl- 1-(oxetan-3-yl)- 1H-pyrazol-5- yl)piperidin-1-yl)- 2-(trifluoromethyl) pyrimidin-4- yl)azetidin-3- yl)piperazin-1- yl)prop-2-en-1-one | 2-136 | 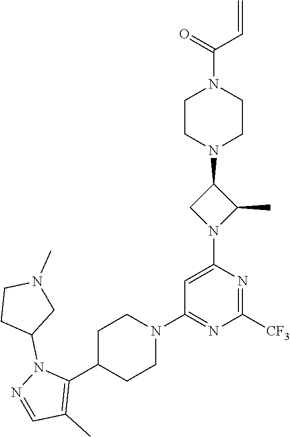 [SMILES: C=CC(=O)N1CCN([C@@H]2CN(c3cc(N4CCC(c5c(C)cnn5C5CCN(C)C5)CC4)nc(C(F)(F)F)n3)[C@@H]2C)CC1] | 1-(4-((2R,3R)-2- methyl-1-(6-(4-(4- methyl-1-(1- methylpyrrolidin- 3-yl)-1H-pyrazol- 5-yl)piperidin-1- yl)-2- (trifluoromethyl) pyrimidin-4- yl)azetidin-3- yl)piperazin-1- yl)prop-2-en-1-one |
| 2-133 | 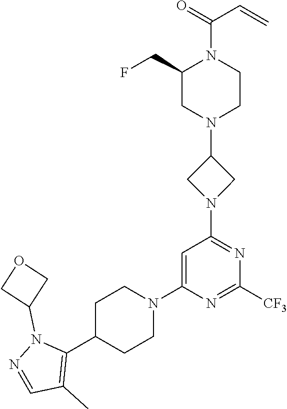 [SMILES: C=CC(=O)N1CCN(C2CN(c3cc(N4CCC(c5c(C)cnn5C5COC5)CC4)nc(C(F)(F)F)n3)C2)C[C@H]1CF] | (S)-1-(2- (fluoromethyl)-4- (1-(6-(4-(4-methyl- 1-(oxetan-3-yl)- 1H-pyrazol-5- yl)piperidin-1-yl)- 2-(trifluoromethyl) pyrimidin-4- yl)azetidin-3- yl)piperazin-1- yl)prop-2-en-1-one | 2-137 | 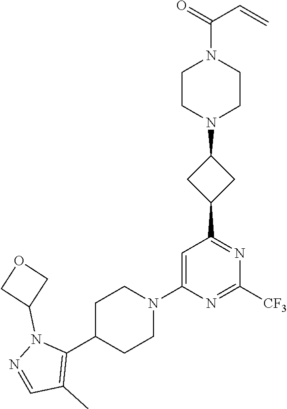 [SMILES: C=CC(=O)N1CCN([C@H]2C[C@@H](c3cc(N4CCC(c5c(C)cnn5C5COC5)CC4)nc(C(F)(F)F)n3)C2)CC1] | 1-(4-((1S,3S)-3-(6- (4-(4-methyl-1- (oxetan-3-yl)-1H- pyrazol-5- yl)piperidin-1-yl)- 2-(trifluoromethyl) pyrimidin-4- yl)cyclobutyl) piperazan-1- yl)prop-2- en-1-one |
| 2-134 | 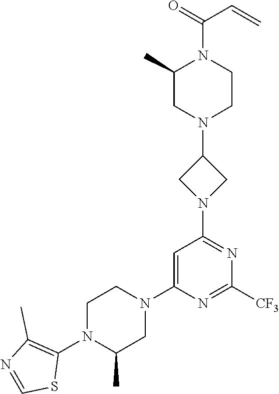 [SMILES: C=CC(=O)N1CCN(C2CN(c3cc(N4CCN(c5scnc5C)[C@H](C)C4)nc(C(F)(F)F)n3)C2)C[C@H]1C] | 1-((2R)-2-methy]- 4-(1-(6-((3S)-3- methyl-4-(4- methylthiazol-5- yl)piperazin-1-yl)- 2-(trifluoromethyl) pyrimidin-1- yl)azetidin-3- yl)piperazin-1- yl)prop-2-en-1-one | 2-138 | 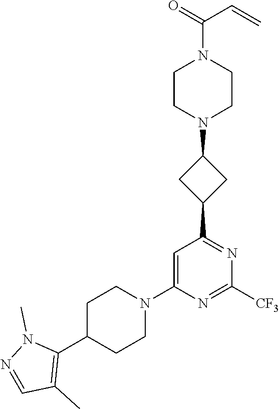 [SMILES: C=CC(=O)N1CCN([C@H]2C[C@@H](c3cc(N4CCC(c5c(C)cnn5C)CC4)nc(C(F)(F)F)n3)C2)CC1] | 1-(4-((1R,3R)-3-(6- (4-(1,4-dimethyl- 1H-pyrazol-5- yl)piperidin-1-yl)- 2-(trifluoromethyl) pyrimidin-4- yl)cyclobutyl) piperazin-l- yl)prop-2- en-1-one |
| 2-139 | 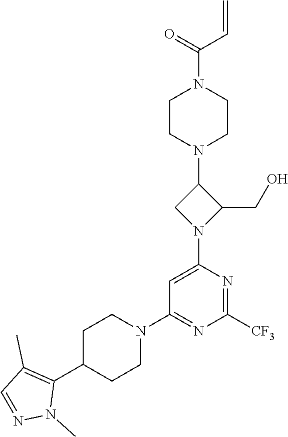 [SMILES: C=CC(=O)N1CCN(C2CN(c3cc(N4CCC(c5c(C)cnn5C)CC4)nc(C(F)(F)F)n3)C2CO)CC1] | 1-(4-(1-(6-(4-(1,4- Dimethyl-1H- pyrazol-5- yl)piperidin-1-yl)- 2-(trifluoromethyl) pyrimidin-4-yl)-2- (hydroxymethyl) azetidin-3- yl)piperazin-1- yl)prop-2-en-1-one | 2-140-1 | 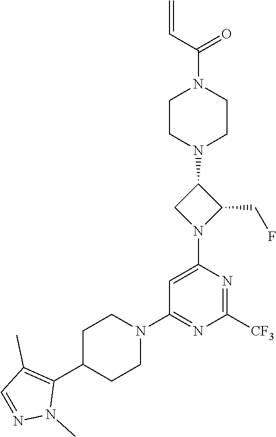 [SMILES: C=CC(=O)N1CCN([C@H]2CN(c3cc(N4CCC(c5c(C)cnn5C)CC4)nc(C(F)(F)F)n3)[C@H]2CF)CC1] | 1-(4-((2R,3S)-1-(6- (4-(1,4-dimethyl- 1H-pyrazol-5- yl)piperidin-1-yl)- 2-(trifluoromethyl) pyrimidin-4-yl)-2- (fluoromethyl) azetidin-3- yl)piperazin- 1-yl)prop-2-en-1- one |
| 2-139-1 | 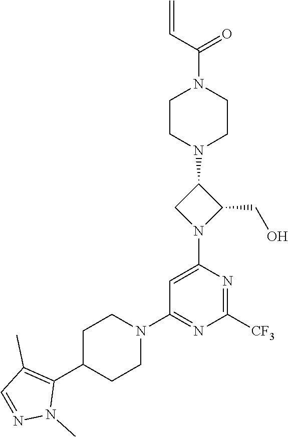 [SMILES: C=CC(=O)N1CCN([C@H]2CN(c3cc(N4CCC(c5c(C)cnn5C)CC4)nc(C(F)(F)F)n3)[C@H]2CO)CC1] | 1-(4-((2R,3.5)-1-(6- (4-(1,4-dimethyl- 1H-pyrazol-5- yl)piperidin-1-yl)- 2-(trifluoromethyl) pyrimidin-4-yl)-2- (hydroxymethyl) azetidin-3- yl)piperazin-1- yl)prop-2-en-1-one | 2-140-2 | 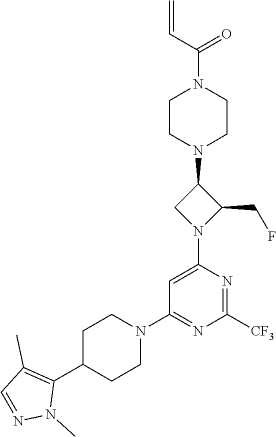 [SMILES: C=CC(=O)N1CCN([C@@H]2CN(c3cc(N4CCC(c5c(C)cnn5C)CC4)nc(C(F)(F)F)n3)[C@@H]2CF)CC1] | 1-(4-((2S,3R)-1-(6- (4-(1,4-dimethyl- 1H-pyrazol-5- yl)piperidin-1-yl)- 2-(trifluoromethyl) pyrimidin-4-yl)-2- (fluoromethyl) azetidin-3- yl)piperazin- 1-yl)prop-2-en-1- one |
| 2-139-2 | 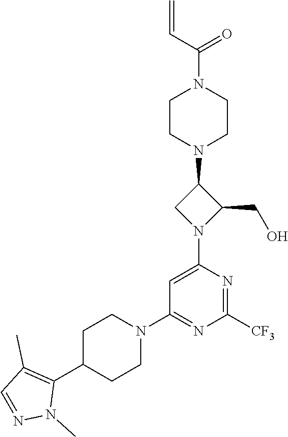 [SMILES: C=CC(=O)N1CCN([C@@H]2CN(c3cc(N4CCC(c5c(C)cnn5C)CC4)nc(C(F)(F)F)n3)[C@@H]2CO)CC1] | 1-(4-((2S,3R)-1-(6- (4-(1,4-dimethy]- 1H-pyrazol-5- yl)piperidin-1-yl)- 2-(trifluoromethyl) pyrimidin-4-yl)-2- (hydroxymethyl) azetidin-3- yl)piperazin-1- yl)prop-2-en-1-one | 2-141 | 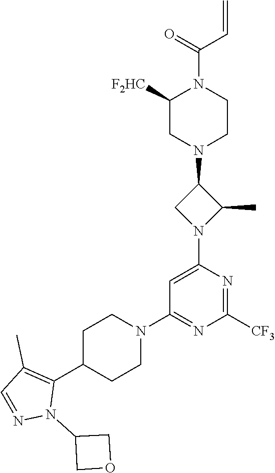 [SMILES: C=CC(=O)N1CCN([C@@H]2CN(c3cc(N4CCC(c5c(C)cnn5C5COC5)CC4)nc(C(F)(F)F)n3)[C@@H]2C)C[C@H]1C(F)F] | 1-((2S)-2- (difluorometliy])-4- ((2R,3R)-2-methyl- 1-(6-(4-(4-methyl- 1-(3-oxetanyl)-1H- pyrazol-5-yl)-1- piperidinyl)-2- (trifluoromethyl)- 4-pyrimidinyl)-3- azetidinyl)-1- piperazinyl)-2- propeut-1-one |
| 2-140 | 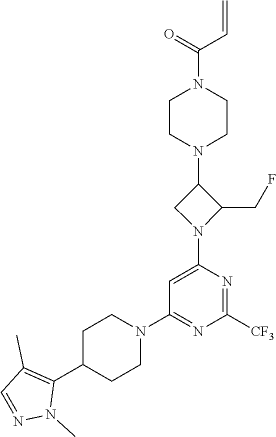 [SMILES: C=CC(=O)N1CCN(C2CN(c3cc(N4CCC(c5c(C)cnn5C)CC4)nc(C(F)(F)F)n3)C2CF)CC1] | 1-(4-(1-(6-(4-(1,4- Dimethyl-1H- pyrazol-5- yl)piperidin-1-yl)- 2-(trifluoromethyl) pyrimidin-4-yl)-2- (fluoromethyl) azetidin-3- yl)piperazin- 1-yl)prop-2-en-1- one | 2-142 | 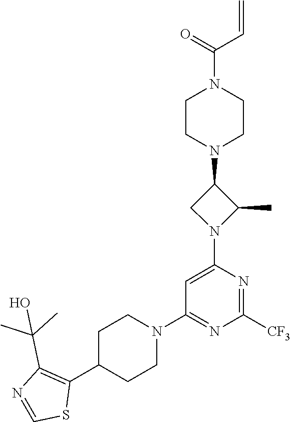 [SMILES: C=CC(=O)N1CCN([C@@H]2CN(c3cc(N4CCC(c5scnc5C(C)(C)O)CC4)nc(C(F)(F)F)n3)[C@@H]2C)CC1] | 1-(4-((2R,3R)-1-(6- (4-(4-(2-hydroxy- 2-propanyl)-1,3- thiazol-5-yl)-1- piperidinyl)-2- (trifluoromethyl)- 4-pyrimidinyl)-2- methyl-3- azetidinyl)-1- piperazinyl)-2- propen-l-one |
| 2-143 | 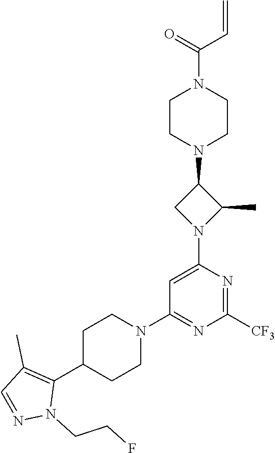 [SMILES: C=CC(=O)N1CCN([C@@H]2CN(c3cc(N4CCC(c5c(C)cnn5CCF)CC4)nc(C(F)(F)F)n3)[C@@H]2C)CC1] | 1-(4-((2R,3R)-1-(6- (4-(1-(2- fluoroethyl)-4- methyl-1H- pyrazol-5-yl)-1- piperidinyl)-2- (trifluoromethyl)- 4-pyrimidiny])-2- methyl-3- azetidinyl)-1- piperazinyl)-2- propen-1-one | 2-147 | 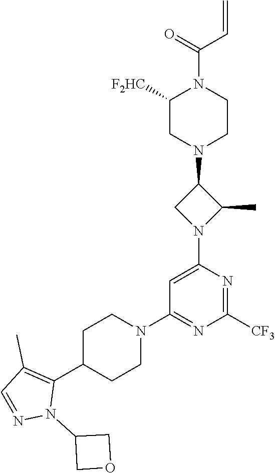 [SMILES: C=CC(=O)N1CCN([C@@H]2CN(c3cc(N4CCC(c5c(C)cnn5C5COC5)CC4)nc(C(F)(F)F)n3)[C@@H]2C)C[C@@H]1C(F)F] | 1-((2R)-2- (difluoromethyl)-4- ((2R,3R)-2-methyl- 1-(6-(4-(4-methyl- 1-(3-oxetanyl)-1H- pyrazol-5-yl)-1- piperidinyl)-2- (trifluoromethyl)- 4-pyrimidinyl)-3- azetidinyl)-1- piperazinyl)-2- propen-1-one |
| 2-144 | 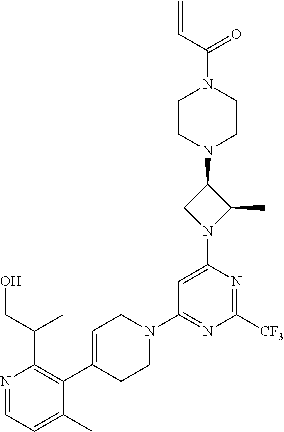 [SMILES: C=CC(=O)N1CCN([C@@H]2CN(c3cc(N4CC=C(c5c(C)ccnc5C(C)CO)CC4)nc(C(F)(F)F)n3)[C@@H]2C)CC1] | 1-(4-((2R,3R)-1-(6- (2-(1- hydroxypropan-2- yl)-4-methyl-3′,6′ dihydro-[3,4′- bipyridin]-1′(2′H)- yl)-2- (trifluoromethyl) pyrimidin-4-yl)-2- methylazetidin-3- yl)piperazin-1- yl)prop-2-en-1-one | 2-148 | 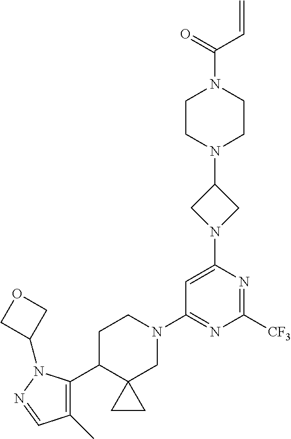 [SMILES: C=CC(=O)N1CCN(C2CN(c3cc(N4CCC(c5c(C)cnn5C5COC5)C5(CC5)C4)nc(C(F)(F)F)n3)C2)CC1] | 1-(4-(1-(6-((8R)-8- (4-methyl-1-(3- oxetanyl)-1H- pyrazol-5-yl)-5- azaspiro\|2.5]octan- 5-yl)-2- (trifluoromethyl)- 4-pyrimidinyl)-3- azetidinyl)-1- piperaziny])-2- propen-1-one |
| 2-145 | 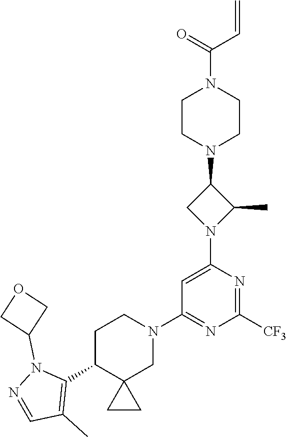 [SMILES: C=CC(=O)N1CCN([C@@H]2CN(c3cc(N4CC[C@@H](c5c(C)cnn5C5COC5)C5(CC5)C4)nc(C(F)(F)F)n3)[C@@H]2C)CC1] | 1-(4-((2R.3R)-2- methyl-1-(6-((8R)- 8-(4-methyl-1-(3- oxetanyl)-1H- pyrazol-5-yl)-5- azaspiro[2.5] octan-5-yl)-2- (trifluoromethyl)- 4-pyrimidinyl)-3- azetidinyl)-1- piperazinyl)-2- propen-1-one | 2-149 | 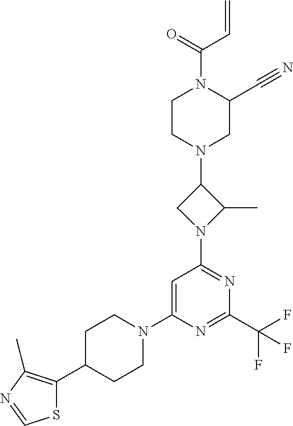 [SMILES: C=CC(=O)N1CCN(C2CN(c3cc(N4CCC(c5scnc5C)CC4)nc(C(F)(F)F)n3)C2C)CC1C#N] | 4-(2-methyl-1-(6- (4-(4-methyl-1,3- thiazol-5-yl)-1- piperidinyl)-2- (trifluoromethyl)- 4-pyrimidinyl)-3- azetidinyl)-1-(2- propenoyl)-2- piperazine- carbonitrile |
| 2-146 | 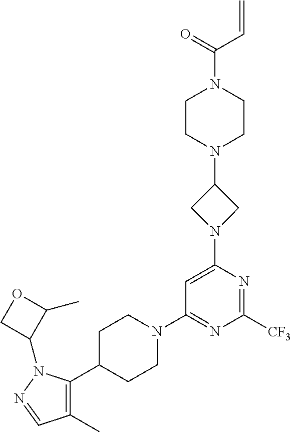 [SMILES: C=CC(=O)N1CCN(C2CN(c3cc(N4CCC(c5c(C)cnn5C5COC5C)CC4)nc(C(F)(F)F)n3)C2)CC1] | 1-(4-(1-(6-(4-(4- methyl-1-(2- methyl-3- oxetanyl)-1H- pyrazol-5-yl)-1- piperidinyl)-2- (trifluoromethyl)- 4-pyrimidinyl)-3- azetidinyl)-1- piperazinyl)-2- propen-1-one | 2-150 | 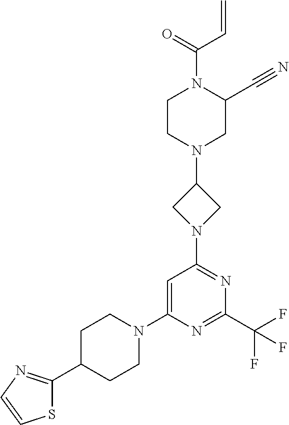 [SMILES: C=CC(=O)N1CCN(C2CN(c3cc(N4CCC(c5nccs5)CC4)nc(C(F)(F)F)n3)C2)CC1C#N] | 1-(2-propenoyl)-4- (1-(6-(4-(1,3- thiazol-2-yl)-1- piperidinyl)-2- (trifluoromethyl)- 4-pyrimidinyl)-3- azetidinyl)-2- piperazine- carbonitrile |
| 2-151 | 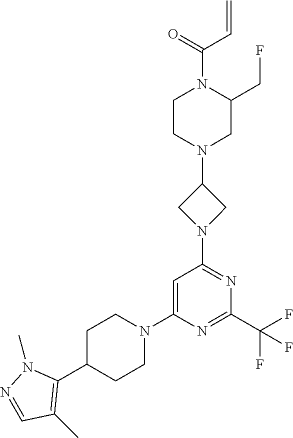 [SMILES: C=CC(=O)N1CCN(C2CN(c3cc(N4CCC(c5c(C)cnn5C)CC4)nc(C(F)(F)F)n3)C2)CC1CF] | 1-(4-(1-(6-(4-(1,4- dimethyl-1H- pyrazol-5-yl)-1- piperidinyl)-2- (trifluoromethyl)- 4-pyrimidinyl)-3- azetidiny])-2- (fluoromethyl)-1- piperazinyl)-2- propen-1-one | 2-155 | 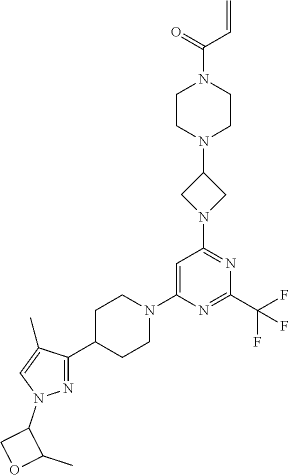 [SMILES: C=CC(=O)N1CCN(C2CN(c3cc(N4CCC(c5nn(C6COC6C)cc5C)CC4)nc(C(F)(F)F)n3)C2)CC1] | 1-(4-(1-(6-(4-(4- methyl-1-(2- methyl-3- oxetanyl)-1H- pyrazol-3-yl)-1- piperidinyl)-2- (trifluoromethyl)- 4-pyrimidinyl)-3- azetidinyl)-1- piperazinyl)-2- propen-1-one |
| 2-152 | 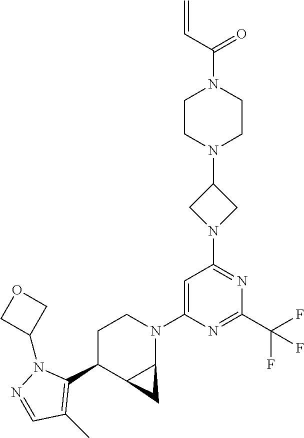 [SMILES: C=CC(=O)N1CCN(C2CN(c3cc(N4CC[C@H](c5c(C)cnn5C5COC5)[C@@H]5C[C@@H]54)nc(C(F)(F)F)n3)C2)CC1] | 1-(4-(1-(6- ((1S,5S,6S)-5-(4- methyl-1-(3- oxetanyl)-1H- pyrazol-5-yl)-2- azabicyclo[4.1.0] heptan-2-yl)-2- (trifluoromethyl)- 4-pyrimidinyl)-3- azetidinyl)-1- piperazinyl)-2- propen-1-one | 2-156 | 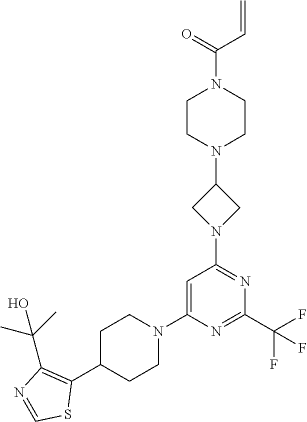 [SMILES: C=CC(=O)N1CCN(C2CN(c3cc(N4CCC(c5scnc5C(C)(C)O)CC4)nc(C(F)(F)F)n3)C2)CC1] | 1-(4-(1-(6-(4-(4-(2- hydroxy-2- propanyl)-1,3- thiazol-5-yl)-1- piperidinyl)-2- (trifluoromethyl)- 4-pyrimidinyl)-3- azetidinyl)-1- piperazinyl)-2- propen-1-one |
| 2-153 | 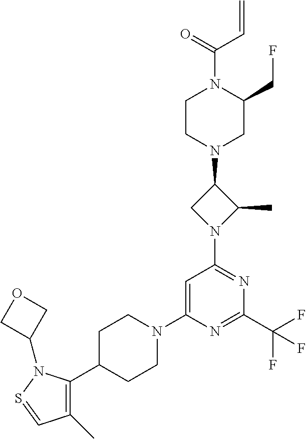 | 1-((2R)-2- (fluoromethyl)-4- ((2R,3R)-2-methyl- 1-(6-(4-(4-methyl- 1-(3-oxetanyl)-1H- pyrazol-5-yl)-1- piperidinyl)-2- (trifluoromethyl)- 4-pyrimidinyl)-3- azetidinyl)-1- piperazinyl)-2- propen-1-one | 2-157 | 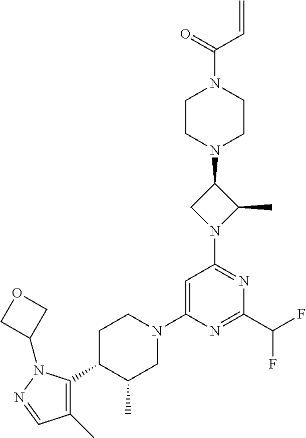 [SMILES: C=CC(=O)N1CCN([C@@H]2CN(c3cc(N4CC[C@@H](c5c(C)cnn5C5COC5)[C@@H](C)C4)nc(C(F)F)n3)[C@@H]2C)CC1] | 1-(4-((2R,3R)-1-(2- (difluorometliyl)-6- ((3R,4R)-3-methyl- 4-(4-methyl-1-(3- oxetanyl)-1H- pyrazol-5-yl)-1- piperidinyl)-4- pyrimidinyl)-2- methyl-3- azetidinyl)-1- piperazinyl)-2- propen-1-one |
| 2-154 | 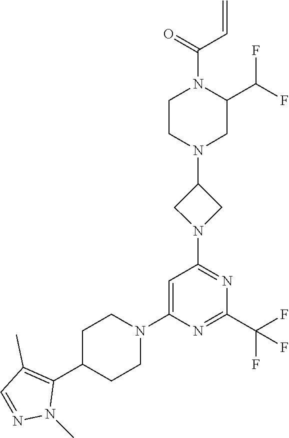 [SMILES: C=CC(=O)N1CCN(C2CN(c3cc(N4CCC(c5c(C)cnn5C)CC4)nc(C(F)(F)F)n3)C2)CC1C(F)F] | 1-12- (difluoromethyl)-4- (1-(6-(4-(1,4- dimethyl-1H- pyrazol-5-yl)-1- piperidinyl)-2- (trifluoromethyl)- 4-pyrimidinyl)-3- azetidinyl)-1- piperazinyl)-2- propen-1-one | 2-158 | 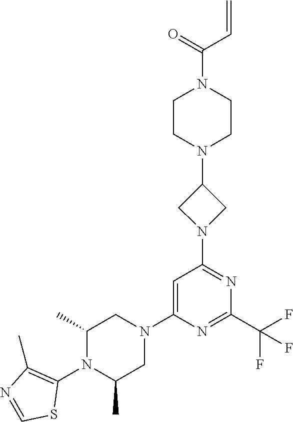 [SMILES: C=CC(=O)N1CCN(C2CN(c3cc(N4C[C@@H](C)N(c5scnc5C)[C@H](C)C4)nc(C(F)(F)F)n3)C2)CC1] | 1-(4-(1-(6- ((3R,5R)-3,5- dimethyl-4-(4- methyl-1,3-thiazol- 5-yl)-1- piperazinyl)-2- (trifluoromethyl)- 4-pyrimidinyl)-3- azetidinyl)-1- piperazinyl)-2- propen-t-one |
| 2-159 | 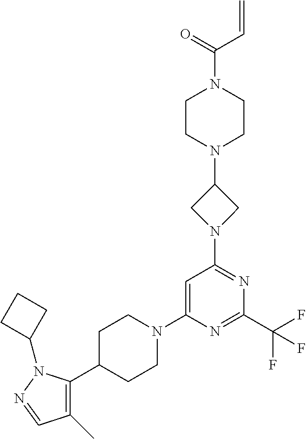 [SMILES: C=CC(=O)N1CCN(C2CN(c3cc(N4CCC(c5c(C)cnn5C5CCC5)CC4)nc(C(F)(F)F)n3)C2)CC1] | 1-(4-(1-(6-(4-(1- cyclobutyl-4- methyl-1H- pyrazol-5-yl)-1- piperidinyl)-2- (trifluoromethyl)- 4-pyrimidiny])-3- azetidinyl)-1- piperazinyl)-2- propen-1-one | 2-163 | 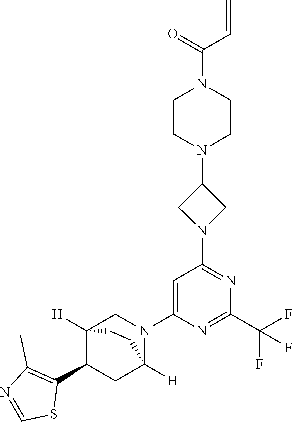 [SMILES: C=CC(=O)N1CCN(C2CN(c3cc(N4C[C@H]5CC[C@@H]4C[C@H]5c4scnc4C)nc(C(F)(F)F)n3)C2)CC1] | 1-(4-(1-(6- ((1R,4S,5S)-5-(4- methyl-1,3-thiazol- 5-yl)-2- azabicyclo[2.2.2] octan-2-yl)-2- (trifluoromethyl)- 4-pyrimidinyl)-3- azetidinyl)-1- piperazinyl)-2- propen-1-one |
| 2-160 | 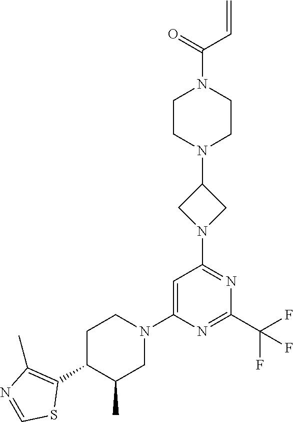 [SMILES: C=CC(=O)N1CCN(C2CN(c3cc(N4CC[C@@H](c5scnc5C)[C@H](C)C4)nc(C(F)(F)F)n3)C2)CC1] | 1-(4-(1-(6- ((3S,4R)-3-methyl- 4-(4-methyl-1,3- thiazol-5-yl)-1- piperidinyl)-2- (trifluoromethyl)- 4-pyrimidinyl)-3- azetidiny])-1- piperazinyl)-2- propen-1-one | 2-164 | 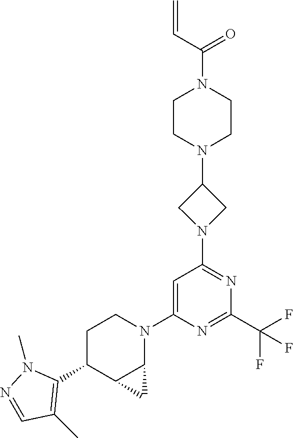 [SMILES: C=CC(=O)N1CCN(C2CN(c3cc(N4CC[C@@H](c5c(C)cnn5C)[C@H]5C[C@H]54)nc(C(F)(F)F)n3)C2)CC1] | 1-(4-(1-(6- ((1R,5R,6R)-5- (1,4-dimethyl-1H- pyrazol-5-yl)-2- azabicyclo[[4.1.0] heptan-2-yl)-2- (trifluoromethyl)- 4-pyrintidinyl)-3- azetidinyl)-1- piperazinyl)-2- propen-1-one |
| 2-161 | 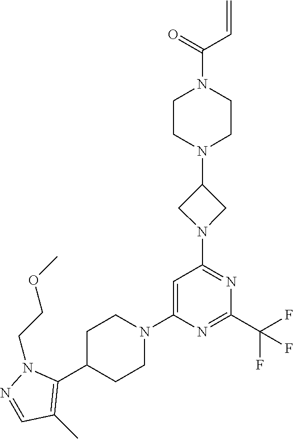 [SMILES: C=CC(=O)N1CCN(C2CN(c3cc(N4CCC(c5c(C)cnn5CCOC)CC4)nc(C(F)(F)F)n3)C2)CC1] | 1-(4-(1-(6-(4-(1-(2- methoxyethyl)-4- methyl-1H- pyrazol-5-yl)-1- piperidinyl)-2- (trifluoromethyl)- 4-pyrimidinyl)-3- azetidiny])-1- piperazinyl)-2- propen-t-one | 2-165 | 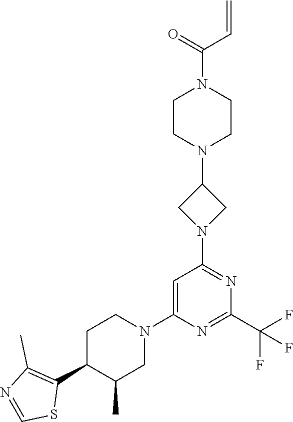 [SMILES: C=CC(=O)N1CCN(C2CN(c3cc(N4CC[C@H](c5scnc5C)[C@H](C)C4)nc(C(F)(F)F)n3)C2)CC1] | 1-(4-(1-(6- ((3S,4S)-3-methyl- 4-(4-methyl-1,3- thiazol-5-yl)-1- piperidinyl)-2- (trifluoromethyl)- 4-pyrimidinyl)-3- azetidinyl)-1- piperazinyl)-2- propon-1-one |
| 2-162 | 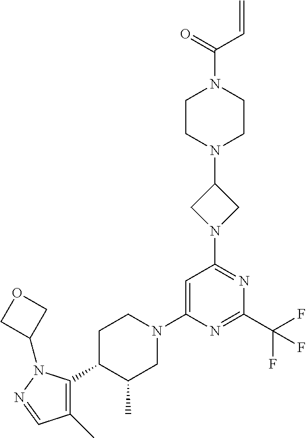 [SMILES: C=CC(=O)N1CCN(C2CN(c3cc(N4CC[C@@H](c5c(C)cnn5C5COC5)[C@@H](C)C4)nc(C(F)(F)F)n3)C2)CC1] | 1-(4-(1-(6- ((3R,4R)-3-methyl- 4-(4-methyl-1-(3- oxetanyl)-1H- pyrazol-5-yl)-1- piperidinyl)-2- (trifluoromethyl)- 4-pyrimidinyl)-3- azetidinyl)-1- piperazinyl)-2- propen-1-one | 2-167 | 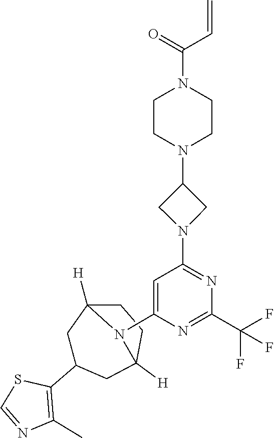 [SMILES: C=CC(=O)N1CCN(C2CN(c3cc(N4C5CCC4CC(c4scnc4C)C5)nc(C(F)(F)F)n3)C2)CC1] | 1-(4-(1-(6-(3-(4- methyl-1,3-thiazol- 5-yl)-3,8- diazabicyclo[3.2.1] octan-8-y])-2- (trifluoromethyl)- 4-pyrimidinyl)-3- azetidinyl)-1- piperazinyl)-2- propen-1-one |
| 2-168 | 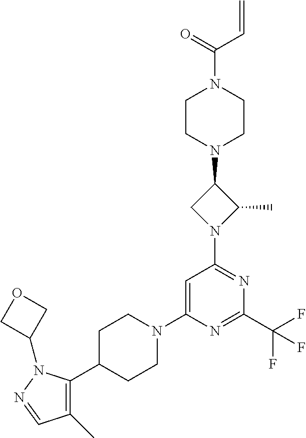 [SMILES: C=CC(=O)N1CCN([C@@H]2CN(c3cc(N4CCC(c5c(C)cnn5C5COC5)CC4)nc(C(F)(F)F)n3)[C@H]2C)CC1] | 1-(4-((2S,3R)-2- methyl-1-16-(4-(4- methyl-1-(3- oxetanyl)-TH- pyrazol-5-yl)-1- piperidinyl)-2- (trifluoromethyl)- 4-pyrimidinyl)-3- azetidinyl)-1- piperazinyl)-2- propen-1-one | 2-172 | 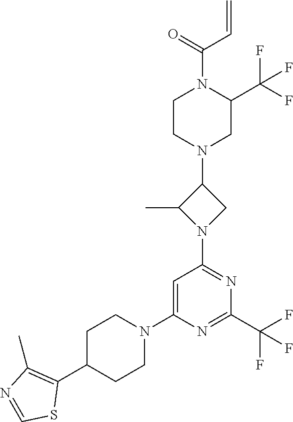 [SMILES: C=CC(=O)N1CCN(C2CN(c3cc(N4CCC(c5scnc5C)CC4)nc(C(F)(F)F)n3)C2C)CC1C(F)(F)F] | 1-(4-(2-methyl-1- (6-(4-(4-methyl- 1,3-thiazol-5-yl)-1- piperidinyl)-2- (trifluoromethyl)- 4-pyrimidinyl)-3- azetidiny])-2- (trifluoromethyl)- 1-piperazinyl)-2- propen-1-one |
| 2-169 | 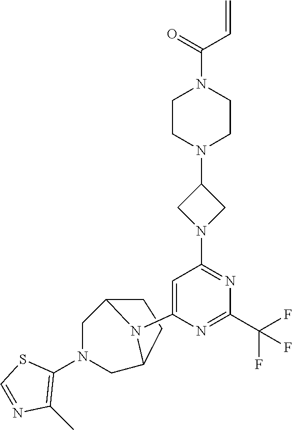 [SMILES: C=CC(=O)N1CCN(C2CN(c3cc(N4C5CCC4CN(c4scnc4C)C5)nc(C(F)(F)F)n3)C2)CC1] | 1-(4-(1-(6-(3-(4- methyl-1,3-thiazol- 5-yl)-8- azabicyclo[3.2.1] octan-8-yl)-2- (trifluoromethyl)- 4-pyrimidinyl)-3- azetidiny])-1- piperazinyl)-2- propen-1-one | 2-173 | 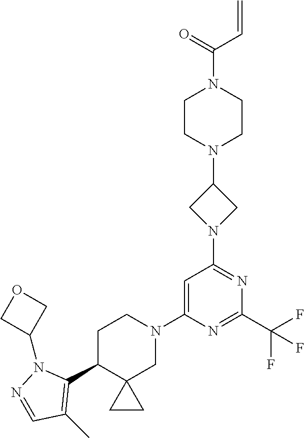 [SMILES: C=CC(=O)N1CCN(C2CN(c3cc(N4CC[C@H](c5c(C)cnn5C5COC5)C5(CC5)C4)nc(C(F)(F)F)n3)C2)CC1] | 1-(4-(1-(6-((8S)-8- (4-methyl-1-(3- oxetanyl)-1H- pyrazol-S-yl)-5- azaspiro[2.5] octan-5-yl)-2- (trifluoromethyl)- 4-pyrimidinyl)-3- azetidinyl)-1- piperaziny])-2- propen-1-one |
| 2-170 | 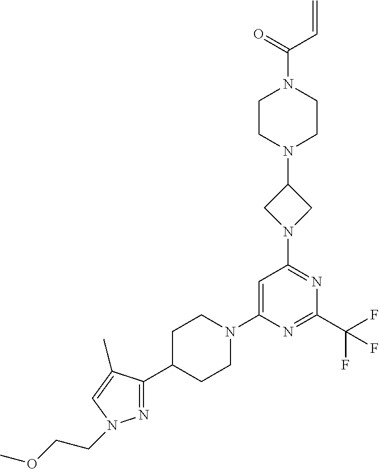 [SMILES: C=CC(=O)N1CCN(C2CN(c3cc(N4CCC(c5nn(CCOC)cc5C)CC4)nc(C(F)(F)F)n3)C2)CC1] | 1-(4-(1-(6-(4-(1-(2- methoxyethyl)-4- methyl-1H- pyrazol-3-yl)-1- piperidinyl)-2- (trifluoromethyl)- 4-pyrimidinyl)-3- azetidiny])-1- piperazinyl)-2- propen-1-one | 2-174 | 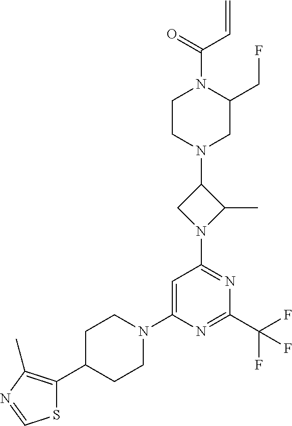 [SMILES: C=CC(=O)N1CCN(C2CN(c3cc(N4CCC(c5scnc5C)CC4)nc(C(F)(F)F)n3)C2C)CC1CF] | 1-(2- (fluoromethyl)-4- (2-methyl-1-(6-(4- (4-methyl-1,3- thiazol-5-yl)-1- piperidinyl)-2- (trifluoromethyl)- 4-pyrimidinyl)-3- azetidinyl)-1- piperazinyl)-2- propen-1-one |
| 2-171 | 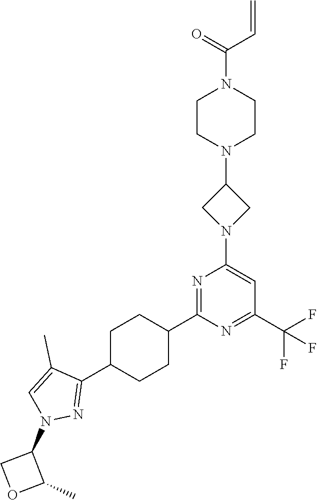 [SMILES: C=CC(=O)N1CCN(C2CN(c3cc(C(F)(F)F)nc(C4CCC(c5nn([C@@H]6CO[C@H]6C)cc5C)CC4)n3)C2)CC1] | 1-(4-(1-(6-(4-(4- methyl-1-((2S,3R)- 2-methyl-3- oxetanyl)-1H- pyrazol-3-yl)-1- piperidinyl)-2- (trifluoromethyl)- 4-pyrimidinyl)-3- azetidinyl)-1- piperazinyl)-2- propen-1-one | 2-175 | 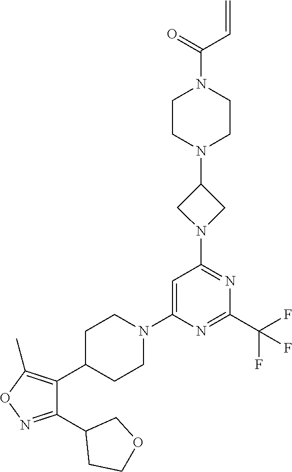 [SMILES: C=CC(=O)N1CCN(C2CN(c3cc(N4CCC(c5c(C6CCOC6)noc5C)CC4)nc(C(F)(F)F)n3)C2)CC1] | 1-(4-(1-(6-(4-(5- methyl-3- (tetraliydro-3- furanyl)-1,2- oxazol-4-yl)-1- piperidinyl)-2- (trifluoromethyl)- 4-pyrimidinyl)-3- azetidinyl)-1- piperazinyl)-2- propen-1-one |
| 2-176 | 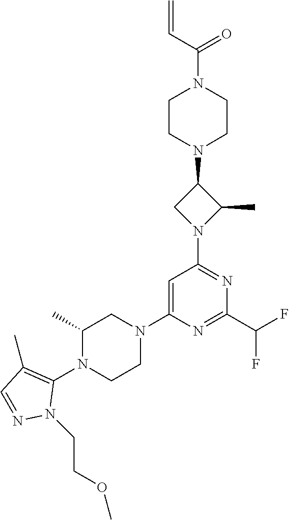 [SMILES: C=CC(=O)N1CCN([C@@H]2CN(c3cc(N4CCN(c5c(C)cnn5CCOC)[C@H](C)C4)nc(C(F)F)n3)[C@@H]2C)CC1] | 1-(4-((2R,3R)-1-(2- (difluoromethyl)-6- ((3R)-4-(1-(2- methoxyethyl)-4- methyl-1H- pyrazol-5-yl)-3- methyl-1- piperazinyl)-4- pyrimidinyl)-2- methyl-3- azetidinyl)-1- piperazinyl)-2- propen-1-one | 2-180 | 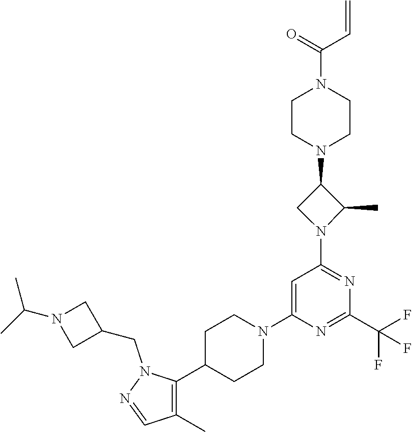 [SMILES: C=CC(=O)N1CCN([C@@H]2CN(c3cc(N4CCC(c5c(C)cnn5CC5CN(C(C)C)C5)CC4)nc(C(F)(F)F)n3)[C@@H]2C)CC1] | 1-(4-((2R,3R)-2- methyl-1-(6-(4-(4- methyl-1-((1-(2- propanyl)-3- azetidinyl)methyl)- 1H-pyrazol-5-yl)- 1-piperidinyl)-2- (trifluoromethyl)- 4-pyrimidinyl)-3- azetidinyl)-1- piperazinyl)-2- propen-1-one |
| 2-177 | 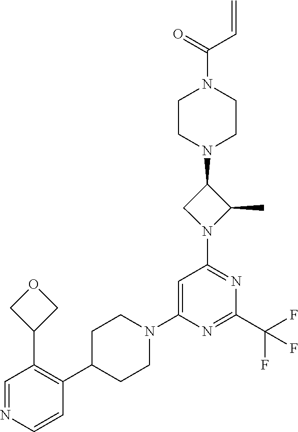 [SMILES: C=CC(=O)N1CCN([C@@H]2CN(c3cc(N4CCC(c5ccncc5C5COC5)CC4)nc(C(F)(F)F)n3)[C@@H]2C)CC1] | 1-(4-((2R,3R)-2- methyl-1-(6-(4-(3- (3-oxetanyl)-4- pyridinyl)-1- piperidinyl)-2- (trifluoromethyl)- 4-pyrimidinyl)-3- azetidiny])-1- piperazinyl)-2- propen-1-one | 2-181 | 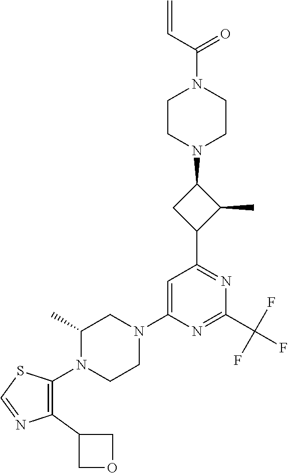 [SMILES: C=CC(=O)N1CCN([C@@H]2CC(c3cc(N4CCN(c5scnc5C5COC5)[C@H](C)C4)nc(C(F)(F)F)n3)[C@@H]2C)CC1] | 1-(4-((2R,3R)-2- methyl-1-(6-((3R)- 3-methyl-4-(4-(3- oxetanyl)-1,3- thiazol-5-yl)-1- piperazinyl)-2- (trifluoromethyl)- 4-pyrimidinyl)-3- azetidinyl)-1- piperazinyl)-2- propen-1-one |
| 2-178 | 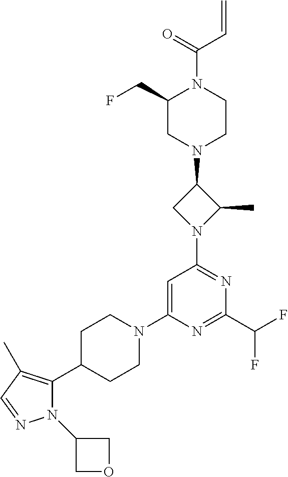 [SMILES: C=CC(=O)N1CCN([C@@H]2CN(c3cc(N4CCC(c5c(C)cnn5C5COC5)CC4)nc(C(F)F)n3)[C@@H]2C)C[C@H]1CF] | 1-((2S)-4- ((2R,3R)-1-(2- (difluoromethyl)-6- (4-(4-methyl-1-(3- oxetanyl)-1H- pyrazol-5-yl)-1- piperidiny])-4- pyrimidinyl)-2- methyl-3- azetidinyl)-2- (fluoromethyl)-1- piperazinyl)-2- propen-1-one | 2-182 | 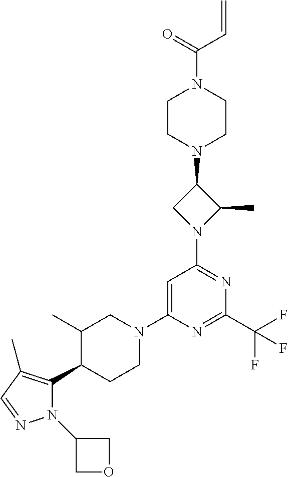 [SMILES: C=CC(=O)N1CCN([C@@H]2CN(c3cc(N4CC[C@@H](c5c(C)cnn5C5COC5)C(C)C4)nc(C(F)(F)F)n3)[C@@H]2C)CC1] | 1-(4-((2R.3R)-2- methyl-1-(6-(3- methyl-4-(4- methyl-1-(3- oxetanyl)-1H- pyrazol-5-yl)-1- piperidinyl)-2- (trifluoromethyl)- 4-pyrimidinyl)-3- azetidinyl)-1- piperazinyl)-2- propen-1-one |
| 2-179 | 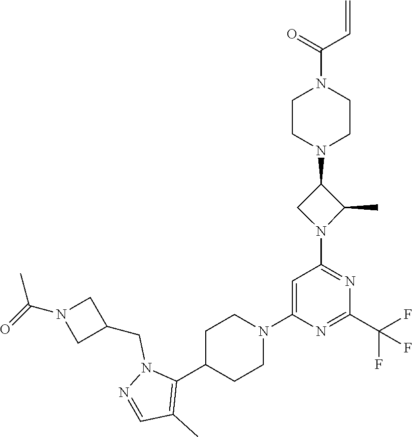 [SMILES: C=CC(=O)N1CCN([C@@H]2CN(c3cc(N4CCC(c5c(C)cnn5CC5CN(C(C)=O)C5)CC4)nc(C(F)(F)F)n3)[C@@H]2C)CC1] | 1-(4-((2R,3R)-1-(6- (4-(1-((1-acctyl-3- azetidinyl)methyl)- 4-methyl-1H- pyrazol-5-yl)-1- piperidinyl)-2- (trifluoromethyl)- 4-pyrimidiny])-2- methyl-3- azetidinyl)-1- piperazinyl)-2- propen-1-one | 2-184 | 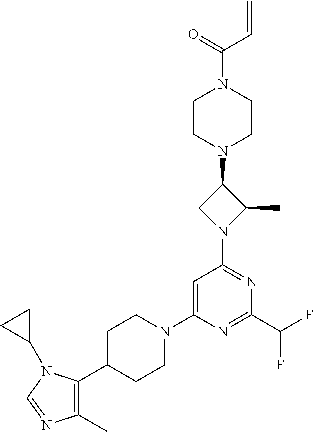 [SMILES: C=CC(=O)N1CCN([C@@H]2CN(c3cc(N4CCC(c5c(C)ncn5C5CC5)CC4)nc(C(F)F)n3)[C@@H]2C)CC1] | 1-(4-((2R,3R)-1-(6- (4-(1-cyclopropyl- 4-methyl-1H- imidazol-5-yl)-1- pipcridinyl)-2- (difluoromethyl)-4- pyrimidinyl)-2- methyl-3- azetidinyl)-1- piperazinyl)-2- propen-t-one |
| 2-185 | 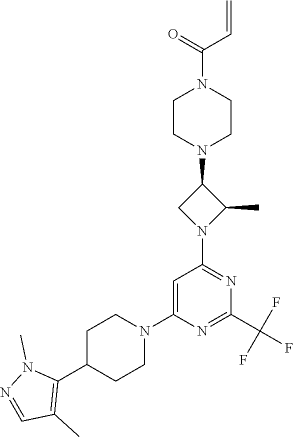 [SMILES: C=CC(=O)N1CCN([C@@H]2CN(c3cc(N4CCC(c5c(C)cnn5C)CC4)nc(C(F)(F)F)n3)[C@@H]2C)CC1] | 1-(4-((2R,3R)-1-(6- (4-(1,4-dimethyl- 1H-imidazol-5-y])- 1-piperidinyl)-2- (trifluoromethyl)- 4-pyrimidinyl)-2- methyl-3- azetidinyl)-1- piperazinyl)-2- propen-1-one | 2-189 | 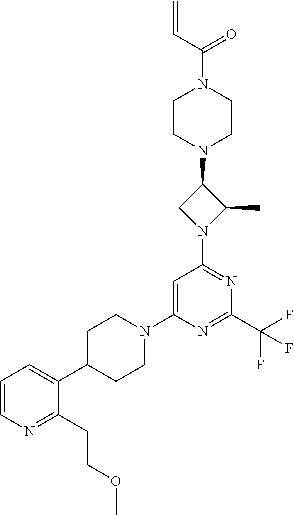 [SMILES: C=CC(=O)N1CCN([C@@H]2CN(c3cc(N4CCC(c5cccnc5CCOC)CC4)nc(C(F)(F)F)n3)[C@@H]2C)CC1] | 1-(4-((2R.3R)-1-(6- (4-(2-(2- methoxyethyl)-3- pyridinyl)-1- piperidiny])-2- (trifluoromethyl)- 4-pyrimidinyl)-2- methyl-3- azetidinyl)-1- piperazinyl)-2- propen-1-one |
| 2-186 | 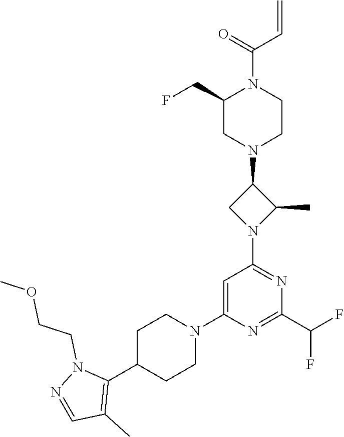 [SMILES: C=CC(=O)N1CCN([C@@H]2CN(c3cc(N4CCC(c5c(C)cnn5CCOC)CC4)nc(C(F)F)n3)[C@@H]2C)C[C@H]1CF] | 1-((2S)-4- ((2R.3R)-1-(2- (difluoromethyl)-6- (4-(1-(2- methoxyethyl)-4- methyl-1H- pyrazol-5-yl)-1- pipcridinyl)-4- pyrimidinyl)-2- methyl-3- azetidinyl)-2- (fluoromethyl)-1- piperazinyl)-2- propen-1-one | 2-190 | 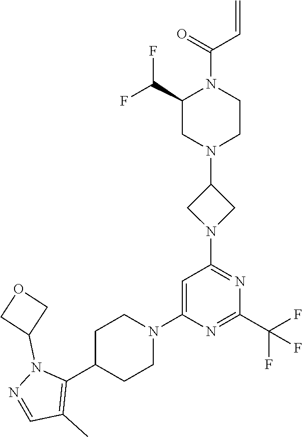 [SMILES: C=CC(=O)N1CCN(C2CN(c3cc(N4CCC(c5c(C)cnn5C5COC5)CC4)nc(C(F)(F)F)n3)C2)C[C@H]1C(F)F] | 1-((2S)-2- (difluoromethyl)-4- (1-(6-(4-(4-methyl- 1-(3-oxetanyl)-1H- pyrazol-5-yl)-1- piperidinyl)-2- (trifluoromethyl)- 4-pyrintidinyl)-3- azetidinyl)-1- piperaziny])-2- propen-1-one |
| 2-187 | 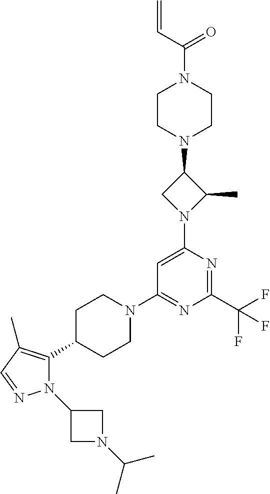 | 1-(4-((2R.3R)-2- methyl-1-(6-(4-(4- methyl-1-(1-(2- propany])-3- azetidinyl)-1H- pyrazol-5-yl)-1- piperidinyl)-2- (trifluoromethyl)- 4-pyrimidinyl)-3- azetidinyl)-1- piperazinyl)-2- propen-1-one | 2-191 | 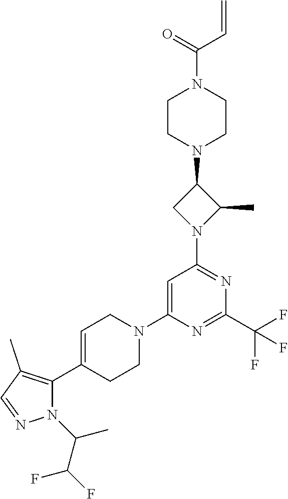 [SMILES: C=CC(=O)N1CCN([C@@H]2CN(c3cc(N4CC=C(c5c(C)cnn5C(C)C(F)F)CC4)nc(C(F)(F)F)n3)[C@@H]2C)CC1] | 1-(4-((2R,3R)-1-(6- (4-(1-(1,1-difluoro- 2-propanyl)-4- methyl-1H- pyrazol-5-yl)-3,6- dihydro-1(2H)- pyridinyl)-2- (trifluoromethyl)- 4-pyrimidinyl)-2- methyl-3- azetidinyl)-1- piperazinyl)-2- propen-1-one |
| 2-188 | 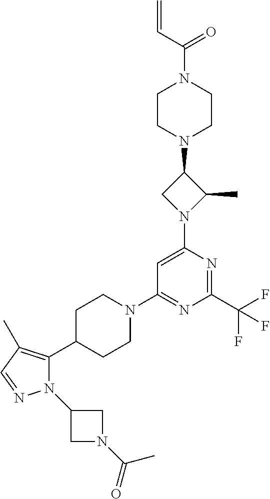 [SMILES: C=CC(=O)N1CCN([C@@H]2CN(c3cc(N4CCC(c5c(C)cnn5C5CN(C(C)=O)C5)CC4)nc(C(F)(F)F)n3)[C@@H]2C)CC1] | 1-(4-((2R,3R)-1-(6- (4-(1-(1-acetyl-3- azetidinyl)-4- methyl-1H- pyrazol-5-yl)-1- piperidinyl)-2- (trifluoromethyl)- 4-pyrimidinyl)-2- methyl-3- azetidiny])-1- piperazinyl)-2- propen-1-one | 2-192 | 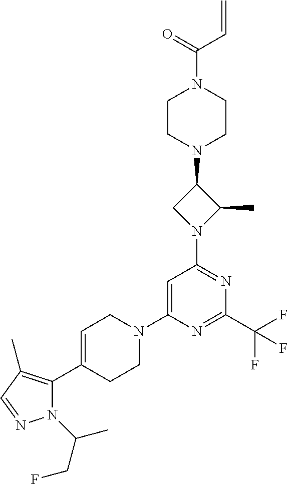 [SMILES: C=CC(=O)N1CCN([C@@H]2CN(c3cc(N4CC=C(c5c(C)cnn5C(C)CF)CC4)nc(C(F)(F)F)n3)[C@@H]2C)CC1] | 1-(4-((2R,3R)-1-(6- (4-(1-(1-fluoro-2- propanyl)-4- methyl-1H- pyrazol-5-yl)-3,6- dihydro-1(2H)- pyridinyl)-2- (trifluoromethyl)- 4-pyrimidinyl)-2- methyl-3- azetidinyl)-1- piperaziny])-2- propen-1-one |
| 2-193 | 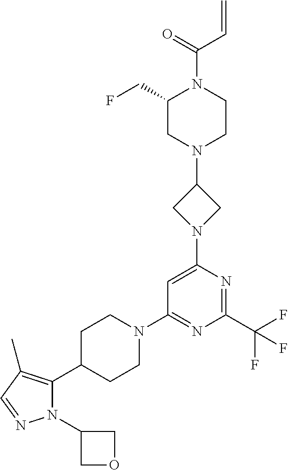 [SMILES: C=CC(=O)N1CCN(C2CN(c3cc(N4CCC(c5c(C)cnn5C5COC5)CC4)nc(C(F)(F)F)n3)C2)C[C@@H]1CF] | 1-((2R)-2- (fluoromethyl)-4- (1-(6-(4-(4-methyl- 1-(3-oxetanyl)-1H- pyrazol-5-yl)-1- piperidinyl)-2- (trifluoromethyl)- 4-pyrimidinyl)-3- azetidinyl)-1- piperazinyl)-2- propen-1-one | 2-197 | 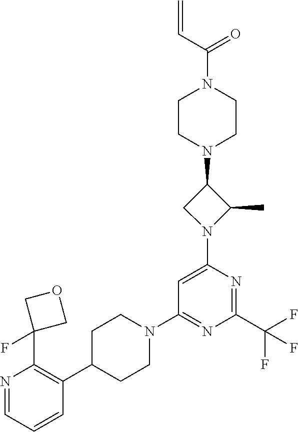 [SMILES: C=CC(=O)N1CCN([C@@H]2CN(c3cc(N4CCC(c5cccnc5C5(F)COC5)CC4)nc(C(F)(F)F)n3)[C@@H]2C)CC1] | 1-(4-((2R,3R)-1-(6- (4-(2-(3-fluoro-3- oxctanyl)-3- pyridinyl)-1- piperidiny])-2- (trifluoromethyl)- 4-pyrimidinyl)-2- methyl-3- azetidinyl)-1- piperazinyl)-2- propen-1-one |
| 2-194 | 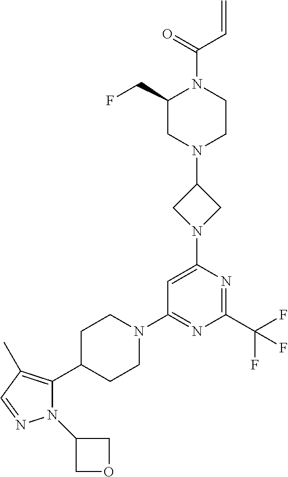 [SMILES: C=CC(=O)N1CCN(C2CN(c3cc(N4CCC(c5c(C)cnn5C5COC5)CC4)nc(C(F)(F)F)n3)C2)C[C@H]1CF] | 1-((2S)-2- (fluoromethyl)-4- (1-(6-(4-(4-methyl- 1-(3-oxetanyl)-1H- pyrazol-5-yl)-1- piperidinyl)-2- (trifluoromethyl)- 4-pyrintidinyl)-3- azetidinyl)-1- piperazinyl)-2- propen-l-one | 2-198 | 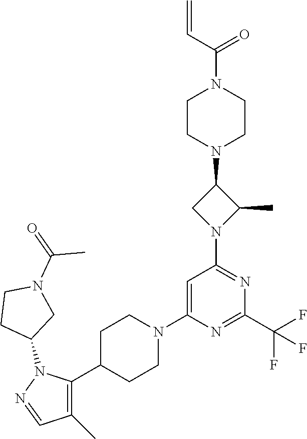 [SMILES: C=CC(=O)N1CCN([C@@H]2CN(c3cc(N4CCC(c5c(C)cnn5[C@@H]5CCN(C(C)=O)C5)CC4)nc(C(F)(F)F)n3)[C@@H]2C)CC1] | 1-(4-((2R,3R)-1-(6- (4-(1-((3R)-1- acetyl-3- pyrrolidinyl)-4- methyl-1H- pyrazol-5-yl)-1- piperidinyl)-2- (trifluoromethyl)- 4-pyrintidinyl)-2- methyl-3- azetidinyl)-1- piperazinyl)-2- propen-1-one |
| 2-195 | 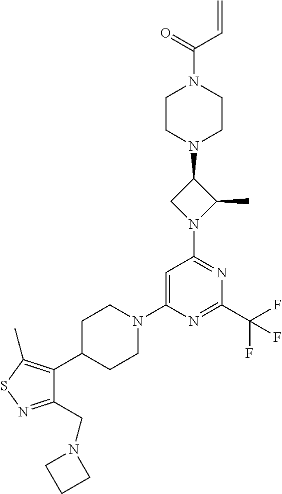 [SMILES: C=CC(=O)N1CCN([C@@H]2CN(c3cc(N4CCC(c5c(CN6CCC6)nsc5C)CC4)nc(C(F)(F)F)n3)[C@@H]2C)CC1] | 1-(4-((2R,3R)-1-(6- (4-(3-(1- azetidinylmethyl)- 5-methyl-1,2- thiazol-4-yl)-1- piperidinyl)-2- (trifluoromethyl)- 4-pyrimidinyl)-2- methyl-3- azetidinyl)-1- piperazinyl)-2- propen-1-one | 2-199 | 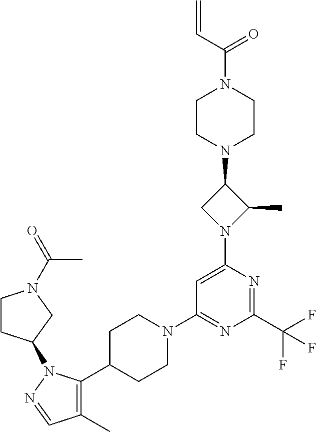 [SMILES: C=CC(=O)N1CCN([C@@H]2CN(c3cc(N4CCC(c5c(C)cnn5[C@H]5CCN(C(C)=O)C5)CC4)nc(C(F)(F)F)n3)[C@@H]2C)CC1] | 1-(4-((2R,3R)-1-(6- (4-(1-((3S)-1- acetyl-3- pyrrolidinyl)-4- methyl-1H- pyrazol-5-yl)-1- piperidiny])-2- (trifluoromethyl)- 4-pyrimidinyl)-2- methyl-3- azetidinyl)-1- piperazinyl)-2- propen-1-one |
| 2-196 | 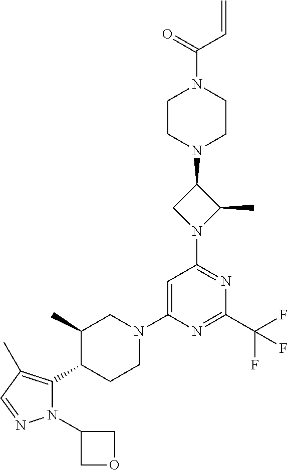 [SMILES: C=CC(=O)N1CCN([C@@H]2CN(c3cc(N4CC[C@H](c5c(C)cnn5C5COC5)[C@@H](C)C4)nc(C(F)(F)F)n3)[C@@H]2C)CC1] | 1-(4-((2R,3R)-2- methyl-1-(6- ((3R,4S)-3-methyl- 4-(4-methyl-1-(3- oxetanyl)-1H- pyrazol-5-yl)-1- piperidiny])-2- (trifluoromethyl)- 4-pyrimidinyl)-3- azetidinyl])-1- piperazinyl)-2- propen-1-one | 2-200 | 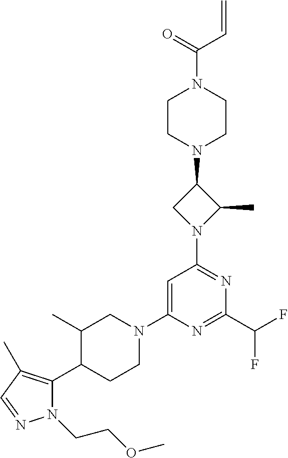 [SMILES: C=CC(=O)N1CCN([C@@H]2CN(c3cc(N4CCC(c5c(C)cnn5CCOC)C(C)C4)nc(C(F)F)n3)[C@@H]2C)CC1] | 1-(4-((2R,3R)-1-(2- (difluoromethyl)-6- (4-(1-(2- methoxyethyl)-4- methyl-1H- pyrazol-5-yl)-3- methyl-1- piperidinyl)-4- pyrimidinyl)-2- methyl-3- azetidinyl)-1- piperazinyl)-2- propen-1-one |
| | mixture of trans isomers at | | | | |
| | piperidine | | | | |
| 2-201 | 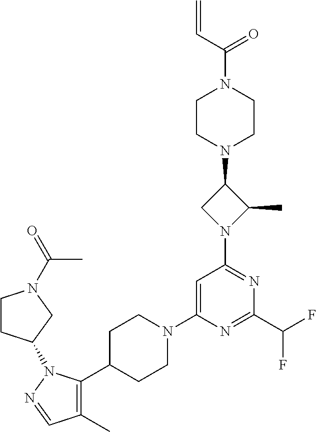 [SMILES: C=CC(=O)N1CCN([C@@H]2CN(c3cc(N4CCC(c5c(C)cnn5[C@@H]5CCN(C(C)=O)C5)CC4)nc(C(F)F)n3)[C@@H]2C)CC1] | 1-(4-((2R,3R)-1-(6- (4-(1-((3R)-1- acetyl-3- pyrrolidinyl)-4- methyl-1H- pyrazol-5-yl)-1- piperidinyl)-2- (difluoromcthyl)-4- pyrimidinyl)-2- methyl-3- azetidinyl)-1- piperazinyl)-2- propen-1-one | 2-205 | 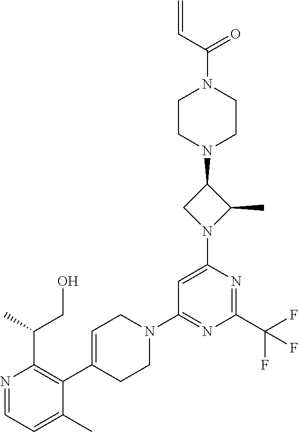 [SMILES: C=CC(=O)N1CCN([C@@H]2CN(c3cc(N4CC=C(c5c(C)ccnc5[C@H](C)CO)CC4)nc(C(F)(F)F)n3)[C@@H]2C)CC1] | 1-(4-((2R,3R)-1-(6- (2-((2S)-1- hydroxy-2- propanyl)-4- methyl-3′,6′- dihydro[3,4′- bipyridin]-1′(2′H)- yl)-2- (trifluoromethyl)- 4-pyrimidinyl)-2- methyl-3- azetidiny])-1- piperazinyl)-2- propen-1-one |
| 2-202 | 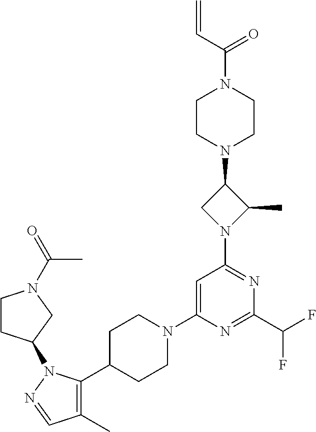 [SMILES: C=CC(=O)N1CCN([C@@H]2CN(c3cc(N4CCC(c5c(C)cnn5[C@H]5CCN(C(C)=O)C5)CC4)nc(C(F)F)n3)[C@@H]2C)CC1] | 1-(4-((2R,3R)-1-(6- (4-(1-((3S)-1- acetyl-3- pyrrolidinyl)-4- methyl-1H- pyrazol-5-yl)-1- piperidinyl)-2- (difluoromethyl)-4- pyrimidinyl)-2- methyl-3- azetidinyl)-1- piperazinyl)-2- propen-1-one | 2-206 | 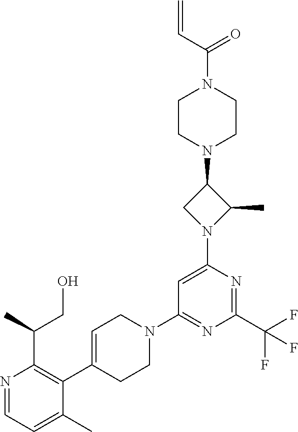 [SMILES: C=CC(=O)N1CCN([C@@H]2CN(c3cc(N4CC=C(c5c(C)ccnc5[C@@H](C)CO)CC4)nc(C(F)(F)F)n3)[C@@H]2C)CC1] | 1-(4-((2R,3R)-1-(6- (2-((2R)-1- hydroxy-2- propanyl)-4- methyl-3′,6′- dihydro[3,4′- bipyridin]-1′(2′H)- yl)-2- (trifluoromethyl)- 4-pyrimidinyl)-2- methyl-3- azetidinyl)-1- piperazinyl)-2- propen-1-one |
| 2-203 | 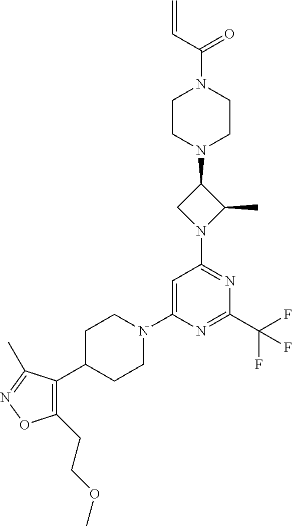 [SMILES: C=CC(=O)N1CCN([C@@H]2CN(c3cc(N4CCC(c5c(C)noc5CCOC)CC4)nc(C(F)(F)F)n3)[C@@H]2C)CC1] | 1-(4-((2R,3R)-1-(6- (4-(5-(2- methoxyethyl)-3- methyl-1,2-oxazol- 4-yl)-1- piperidinyl)-2- (trifluoromethyl)- 4-pyrimidinyl)-2- methyl-3- azetidinyl)-1- piperazinyl)-2- propen-1-one | 2-207 | 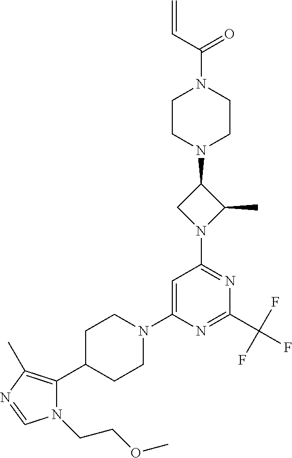 [SMILES: C=CC(=O)N1CCN([C@@H]2CN(c3cc(N4CCC(c5c(C)ncn5CCOC)CC4)nc(C(F)(F)F)n3)[C@@H]2C)CC1] | 1-(4-((2R,3R)-1-(6- (4-(1-(2- methoxyethyl)-4- methyl-1H- imidazol-5-yl)-1- piperidinyl)-2- (trifluoromethyl)- 4-pyrimidinyl)-2- methyl-3- azetidinyl)-1- piperazinyl)-2- propen-1-one |
| 2-204 | 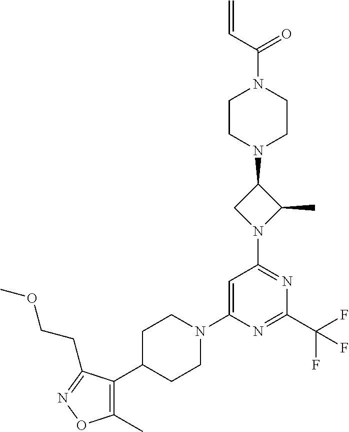 [SMILES: C=CC(=O)N1CCN([C@@H]2CN(c3cc(N4CCC(c5c(CCOC)noc5C)CC4)nc(C(F)(F)F)n3)[C@@H]2C)CC1] | 1-(4-((2R,3R)-1-(6- (4-(3-(2- methoxyethyl)-5- methyl-1,2-oxazol- 4-yl)-1- piperidinyl)-2- (trifluoromethyl)- 4-pyrimidinyl)-2- methyl-3- azetidiny])-1- piperazinyl)-2- propen-1-one | 2-208 | 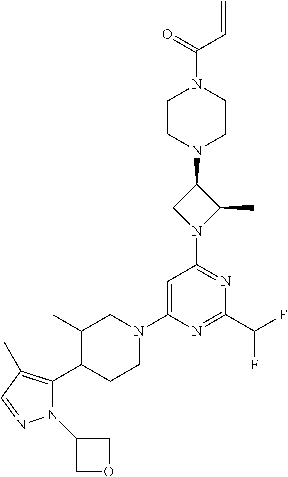 [SMILES: C=CC(=O)N1CCN([C@@H]2CN(c3cc(N4CCC(c5c(C)cnn5C5COC5)C(C)C4)nc(C(F)F)n3)[C@@H]2C)CC1] | 1-(4-((2R,3R)-1-(2- (diflouromethyl)-6- (3-methyl-4-(4- methyl-1-(3- oxetanyl)-1H- pyrazol-5-yl)-4- piperidinyl)-4- pyrimidinyl)-2- methyl-3- azetidinyl)-1- piperazinyl)-2- propen-1-one |
| 2-209 | 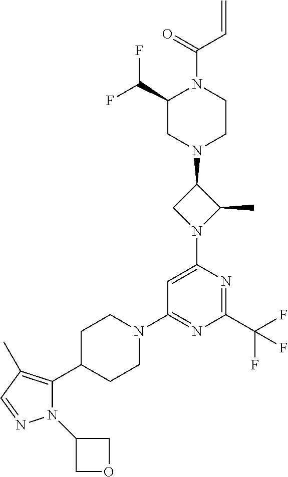 [SMILES: C=CC(=O)N1CCN([C@@H]2CN(c3cc(N4CCC(c5c(C)cnn5C5COC5)CC4)nc(C(F)(F)F)n3)[C@@H]2C)C[C@H]1C(F)F] | 1-((2S)-2- (difluoromethyl)-4- ((2R,3R)-2-methyl- 1-(6-(4-(4-methy]- 1-(3-oxetanyl)-1H- pyrazol-5-yl)-1- piperidinyl)-2- (trifluoromethyl)- 4-pyrimidinyl)-3- azetidinyl)-1- piperazinyl)-2- propen-1-one | 2-213 | 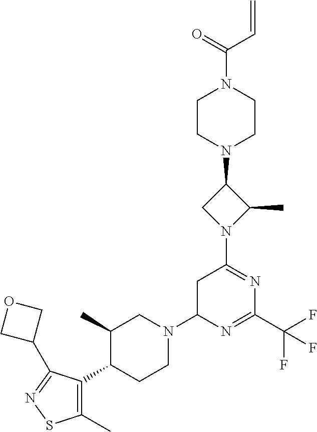 [SMILES: C=CC(=O)N1CCN([C@@H]2CN(C3=NC(C(F)(F)F)=NC(N4CC[C@H](c5c(C6COC6)nsc5C)[C@@H](C)C4)C3)[C@@H]2C)CC1] | 1-(4-((2R,3R)-2- methyl-1-(6- ((3R,4S)-3-methyl- 4-(5-methyl-3-(3- oxetanyl)-1,2- thiazol-4-yl)-1- piperidinyl)-2- (trifluoromethyl)- 4-pyrimidinyl)-3. azetidinyl)-1- piperazinyl)-2- propen-1-one |
| | | | | mixture of trans isomers at | |
| | | | | piperidine | |
| 2-210 | 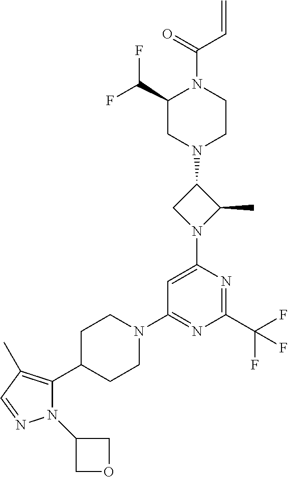 [SMILES: C=CC(=O)N1CCN([C@H]2CN(c3cc(N4CCC(c5c(C)cnn5C5COC5)CC4)nc(C(F)(F)F)n3)[C@@H]2C)C[C@H]1C(F)F] | 1-((2S)-2- (difluoromcthyl)-4- ((2R,3S)-2-methyl- 1-(6-(4-(4-methyl- 1-(3-oxetanyl)-1H- pyrazol-5-yl)-1- piperidinyl)-2- (trifluoromethyl)- 4-pyrimidiny])-3- azetidinyl)-1- piperazinyl)-2- propen-1-one | 2-214 | 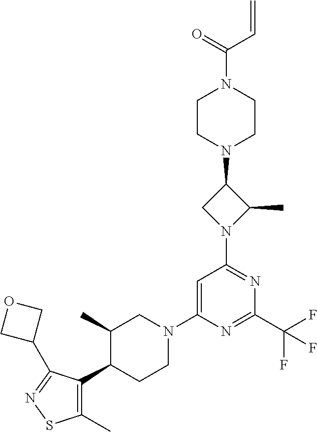 [SMILES: C=CC(=O)N1CCN([C@@H]2CN(c3cc(N4CC[C@@H](c5c(C6COC6)nsc5C)[C@@H](C)C4)nc(C(F)(F)F)n3)[C@@H]2C)CC1] | 1-(4-((2R,3R)-2- methyl-1-(6- ((3R,4R)-3-methyl- 4-(5-methyl-3-(3- oxetanyl)-1,2- thiazol-4-yl)-1- piperidinyl)-2- (trifluoromethyl)- 4-pyrimidinyl)-3- azetidinyl)-1- piperazinyl)-2- propen-1-one |
| 2-211 | 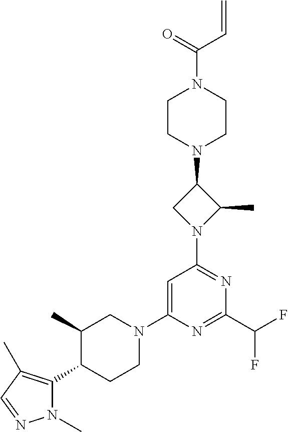 [SMILES: C=CC(=O)N1CCN([C@@H]2CN(c3cc(N4CC[C@H](c5c(C)cnn5C)[C@@H](C)C4)nc(C(F)F)n3)[C@@H]2C)CC1] | 1-(4-((2R,3R)-1-(2- (difluoromethyl)-6- ((3R,4S)-4-(1,4- dimethyl-1H- pyrazol-5-yl)-3- methyl-1- piperidinyl)-4- pyrimidinyl)-2- methyl-3- azetidinyl)-1- piperazinyl)-2- propen-1-one | 2-215 | 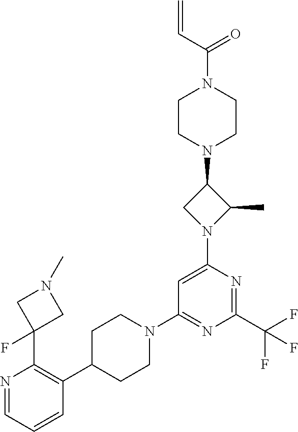 [SMILES: C=CC(=O)N1CCN([C@@H]2CN(c3cc(N4CCC(c5cccnc5C5(F)CN(C)C5)CC4)nc(C(F)(F)F)n3)[C@@H]2C)CC1] | 1-(4-((2R,3R)-1-(6- (4-(2-(3-fluoro-1- methyl-3- azetidinyl)-3- pyridinyl)-1- piperidinyl)-2- (trifluoromethyl)- 4-pyrimidinyl)-2- methyl-3- azetidinyl)-1- piperazinyl)-2- propen-1-one |
| 2-212 | 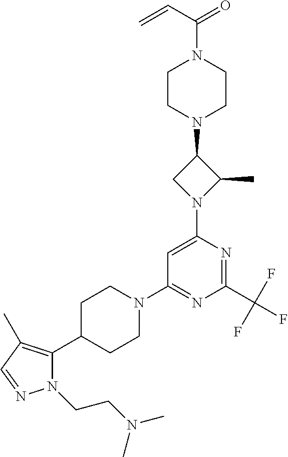 [SMILES: C=CC(=O)N1CCN([C@@H]2CN(c3cc(N4CCC(c5c(C)cnn5CCN(C)C)CC4)nc(C(F)(F)F)n3)[C@@H]2C)CC1] | 1-(4-((2R,3R)-1-(6- (4-(1-(2- (dimethylamino) ethyl)-4-methyl- 1H-pyrazol-5-yl)- 1-piperidinyl)-2- (trifluoromethyl)- methyl-3- azetidiny])-1- piperazinyl)-2- propen-1-one | 2-216 | 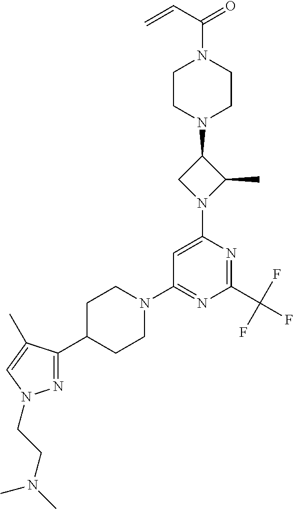 [SMILES: C=CC(=O)N1CCN([C@@H]2CN(c3cc(N4CCC(c5nn(CCN(C)C)cc5C)CC4)nc(C(F)(F)F)n3)[C@@H]2C)CC1] | 1-(4-((2R,3R)-1-(6- (4-(1-(2- (dimethylamino) ethyl)-4-methyl- 1H-pyrazol-3-yl)-1- piperidinyl)-2- (trifluoromethyl)- 4-pyrimidinyl)-2- methyl-3- azetidinyl)-1- piperazinyl)-2- propen-1-one |
| 2-217 | 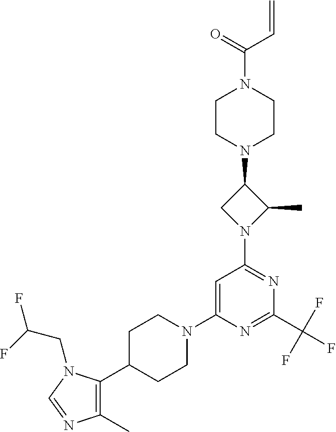 [SMILES: C=CC(=O)N1CCN([C@@H]2CN(c3cc(N4CCC(c5c(C)ncn5CC(F)F)CC4)nc(C(F)(F)F)n3)[C@@H]2C)CC1] | 1-(4-((2R,3R)-1-(6- (4-(1-(2,2- difluoroethyl)-4- methyl-1H- imidazol-5-yl)-1- piperidinyl)-2- (trifluoromethyl)- 4-pyrimidiny])-2- methyl-3- azetidinyl)-1- piperazinyl)-2- propen-1-one | 2-221 | 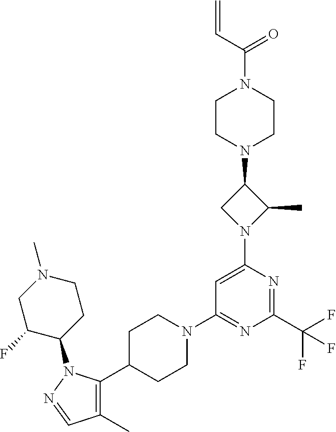 [SMILES: C=CC(=O)N1CCN([C@@H]2CN(c3cc(N4CCC(c5c(C)cnn5[C@@H]5CCN(C)C[C@H]5F)CC4)nc(C(F)(F)F)n3)[C@@H]2C)CC1] | 1-(4-((2R,3R)-1-(6- (4-(1-((3R,4R)-3- fluoro-1-methyl-4- piperidinyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-218 | 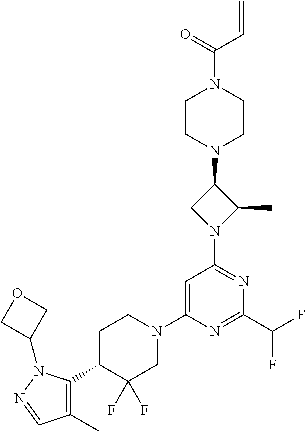 [SMILES: C=CC(=O)N1CCN([C@@H]2CN(c3cc(N4CC[C@@H](c5c(C)cnn5C5COC5)C(F)(F)C4)nc(C(F)F)n3)[C@@H]2C)CC1] | 1-(4-((2R,3R)-1-(2- (difluoromcthyl)-6- ((4S)-3,3-difluoro- 4-(4-methyl-1-(3- oxetanyl)-1H- pyrazol-5-yl)-1- piperidinyl)-4- pyrimidinyl)-2- methyl-3- azetidinyl)-1- piperazinyl)-2- propen-1-one | 2-222 | 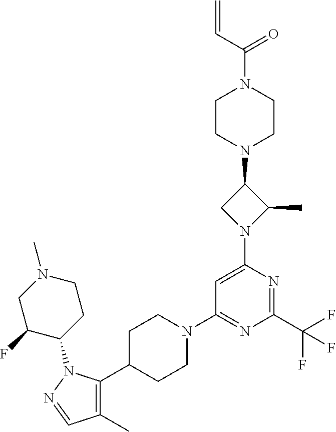 [SMILES: C=CC(=O)N1CCN([C@@H]2CN(c3cc(N4CCC(c5c(C)cnn5[C@H]5CCN(C)C[C@@H]5F)CC4)nc(C(F)(F)F)n3)[C@@H]2C)CC1] | 1-(4-((2R,3R)-1-(6- (4-(1-((3S,4S)-3- fluoro-1-methyl-4- piperidinyl)-4- methyl-1H- pyrazol-5-yl)-1- piperidinyl)-2- (trifluoromethyl)- 4-pyrintidinyl)-2- methyl-3- azetidinyl)-1- piperazinyl)-2- propen-1-one |
| 2-219 | 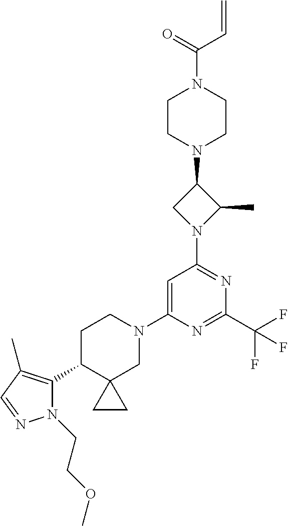 [SMILES: C=CC(=O)N1CCN([C@@H]2CN(c3cc(N4CC[C@@H](c5c(C)cnn5CCOC)C5(CC5)C4)nc(C(F)(F)F)n3)[C@@H]2C)CC1] | 1-(4-((2R,3R)-1-(6- ((8R)-8-(1-(2- methoxyethyl)-4- methyl-1H- pyrazol-5-yl)-5- azaspiro[2.5]octan- 5-yl)-2- (trifluoromethyl)- 4-pyrimidinyl)-2- methyl-3- azetidinyl)-1- piperazinyl)-2- propen-1-one | 2-223 | 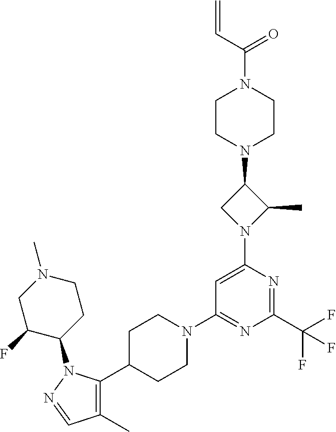 [SMILES: C=CC(=O)N1CCN([C@@H]2CN(c3cc(N4CCC(c5c(C)cnn5[C@@H]5CCN(C)C[C@@H]5F)CC4)nc(C(F)(F)F)n3)[C@@H]2C)CC1] | 1-(4-((2R,3R)-1-(6- (4-(1-((3S,4R)-3- fluoro-1-methyl-4- piperidiny])-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-220 | 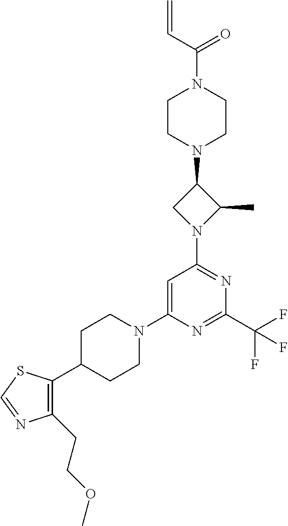 [SMILES: C=CC(=O)N1CCN([C@@H]2CN(c3cc(N4CCC(c5scnc5CCOC)CC4)nc(C(F)(F)F)n3)[C@@H]2C)CC1] | 1-(4-((2R,3R)-1-(6- (4-(4-(2- methoxyethyl)-1,3- thiazol-5-yl)-1- piperidinyl)-2- (trifluoromethyl)- 4-pyrimidinyl)-2- methyl-3- azetidinyl)-1- piperazinyl)-2- propen-1-one | 2-224 | 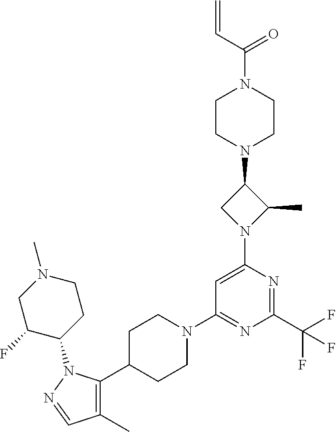 [SMILES: C=CC(=O)N1CCN([C@@H]2CN(c3cc(N4CCC(c5c(C)cnn5[C@H]5CCN(C)C[C@H]5F)CC4)nc(C(F)(F)F)n3)[C@@H]2C)CC1] | 1-(4-((2R,3R)-1-(6- (4-(1-((3R,4S)-3- fluoro-1-methyl-4- piperidinyl)-4- methyl-1H- pyrazol-S-yl)-1- piperidinyl)-2- (trifluoromethyl)- 4-pyrimidinyl)-2- methyl-3- azetidinyl)-1- piperaziny])-2- propen-1-one |
| 2-225 | 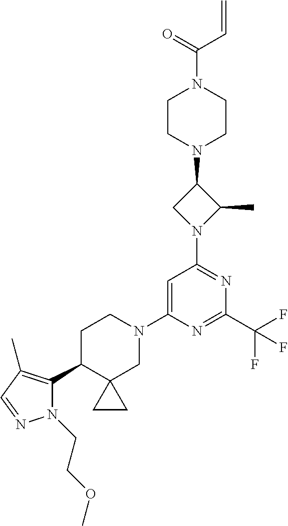 [SMILES: C=CC(=O)N1CCN([C@@H]2CN(c3cc(N4CC[C@H](c5c(C)cnn5CCOC)C5(CC5)C4)nc(C(F)(F)F)n3)[C@@H]2C)CC1] | 1-(4-((2R,3R)-1-(6- ((8S)-8-(1-(2- methoxyethyl)-4- methyl-1H- pyrazol-5-yl)-5- azaspiro[2.5]octan- 5-yl)-2- (trifluoromethyl)- 4-pyrimidinyl)-2- methyl-3- azetidinyl)-1- piperazinyl)-2- propen-1-one | 2-229 | 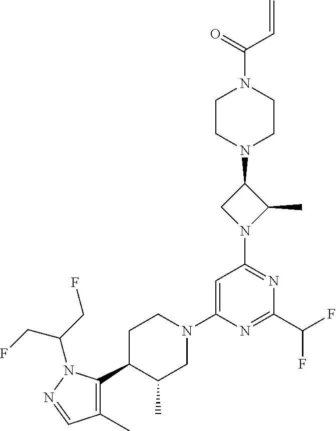 [SMILES: C=CC(=O)N1CCN([C@@H]2CN(c3cc(N4CC[C@H](c5c(C)cnn5C(CF)CF)[C@@H](C)C4)nc(C(F)F)n3)[C@@H]2C)CC1] | 1-(4-((2R,3R)-1-(2- (difluorometlyl)-6- ((3R,4S)-4-(1-(1,3- difluoro-2- propany 1)-4- methyl-1H- pyrazol-5-yl)-3- methyl-1- piperidiny])-4- pyrimidinyl)-2- methyl-3- azetidinyl)-1- piperazinyl)-2- propen-1-one |
| | | | | mixture of trans isomers at | |
| | | | | piperidine | |
| 2-226 | 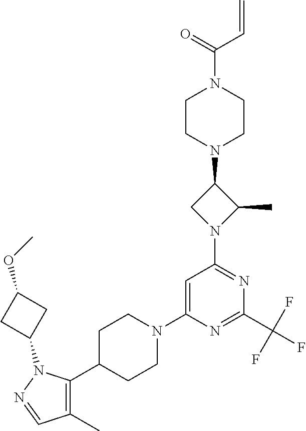 [SMILES: C=CC(=O)N1CCN([C@@H]2CN(c3cc(N4CCC(c5c(C)cnn5[C@H]5C[C@@H](OC)C5)CC4)nc(C(F)(F)F)n3)[C@@H]2C)CC1] | 1-(4-((2R,3R)-1-(6- (4-(1-(cis-3- methoxycyclo- butyl)-4-methyl- 1H-pyrazol-5-yl)- 1-piperidinyl)-2- (trifluoromethyl)- 4-pyrimidinyl)-2- methyl-3- azetidinyl)-1- piperazinyl)-2- propen-1-one | 2-230 | 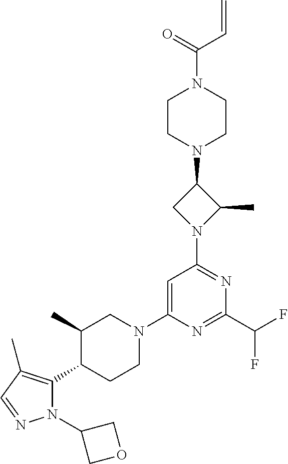 [SMILES: C=CC(=O)N1CCN([C@@H]2CN(c3cc(N4CC[C@H](c5c(C)cnn5C5COC5)[C@@H](C)C4)nc(C(F)F)n3)[C@@H]2C)CC1] | 1-(4-((2R,3R)-1-(2- (difluoromcthyl)-6- ((3R,4S)-3-methyl- 4-(4-methyl-1-(3- oxetanyl)-1H- pyrazol-5-yl)-1- piperidinyl)-4- pyrimidinyl)-2- methyl-3- azetidinyl)-1- piperazinyl)-2- propen-1-one |
| | | | | mixture of trans isomers at | |
| | | | | piperidine | |
| 2-227 | 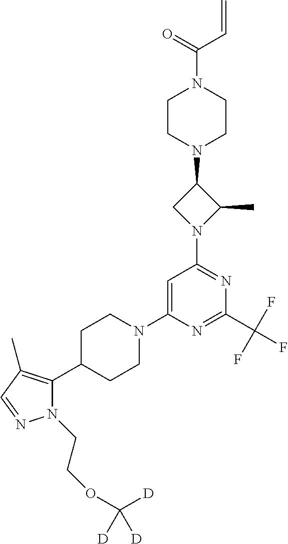 [SMILES: [2H]C([2H])([2H])OCCn1ncc(C)c1C1CCN(c2cc(N3C[C@@H](N4CCN(C(=O)C=C)CC4)[C@H]3C)nc(C(F)(F)F)n2)CC1] | 1-(4-((2R,3R)-1-(6- (4-(1-(2-(methoxy- d3)ethyl)-4- methyl-1H- pyrazol-5- yl)piperidin-1-yl)- 2-(trifluoromethyl) pyrimidin-4-yl)-2- methylazetidin-3- yl)piperazin-1- yl)prop-2-en-1-one | 2-231 | 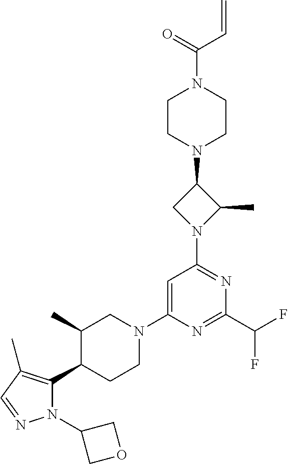 [SMILES: C=CC(=O)N1CCN([C@@H]2CN(c3cc(N4CC[C@@H](c5c(C)cnn5C5COC5)[C@@H](C)C4)nc(C(F)F)n3)[C@@H]2C)CC1] | 1-(4-((2R,3R)-1-(2- (difluorometliyl)-6- ((3R,4R)-3-methyl- 4-(4-methyl-1-(3- oxetanyl)-1H- pyrazol-5-yl)-1- piperidinyl)-4- pyrimidinyl)-2- methyl-3- azetidinyl)-1- piperazinyl)-2- propen-1-one |
| | | | | mixture of trans isomers at piperidine | |
| 2-228 | 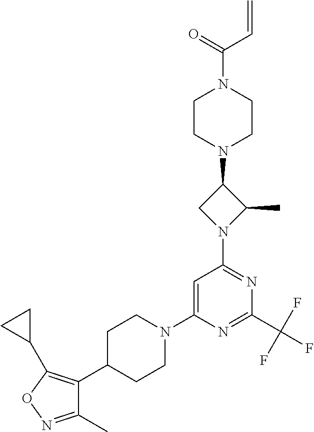 [SMILES: C=CC(=O)N1CCN([C@@H]2CN(c3cc(N4CCC(c5c(C)noc5C5CC5)CC4)nc(C(F)(F)F)n3)[C@@H]2C)CC1] | 1-(4-((2R,3R)-1-(6- (4-(5-cyclopropyl- 3-methyl-1,2- oxazol-4-yl)-1- piperidinyl)-2- (trifluoromethyl)- 4-pyrimidinyl)-2- methyl-3- azetidinyl)-1- piperazinyl)-2- propen-1-one | 2-232 | 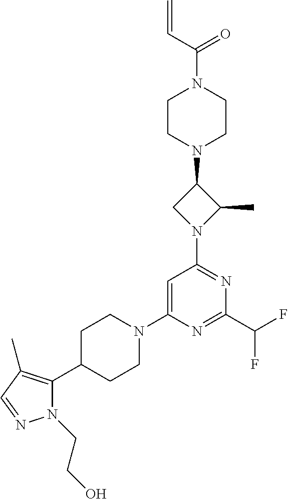 [SMILES: C=CC(=O)N1CCN([C@@H]2CN(c3cc(N4CCC(c5c(C)cnn5CCO)CC4)nc(C(F)F)n3)[C@@H]2C)CC1] | 1-(4-((2R,3R)-1-(2- (difluoromethy])-6- (4-(1-(2- bydroxyethyl)-4- methyl-1H- pyrazol-5-yl)-1- piperidiny])-4- pyrimidinyl)-2- methyl-3- azetidinyl)-1- piperazinyl)-2- propen-1-one |
| 2-233 | 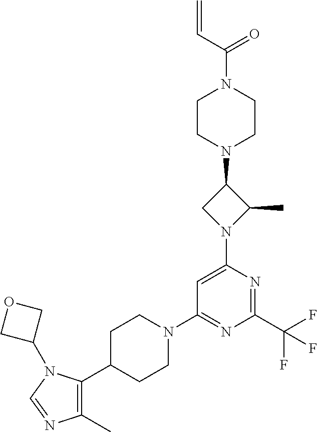 [SMILES: C=CC(=O)N1CCN([C@@H]2CN(c3cc(N4CCC(c5c(C)ncn5C5COC5)CC4)nc(C(F)(F)F)n3)[C@@H]2C)CC1] | 1-(4-((2R,3R)-2- methyl-1-(6-(4-(4- methyl-1-(3- oxetanyl)-1H- imidazol-S-yl)-1- piperidinyl)-2- (trifluoromcthyl)- 4-pyrimidinyl)-3- azetidinyl)-1- piperazinyl)-2- propen-1-one | 2-237 | 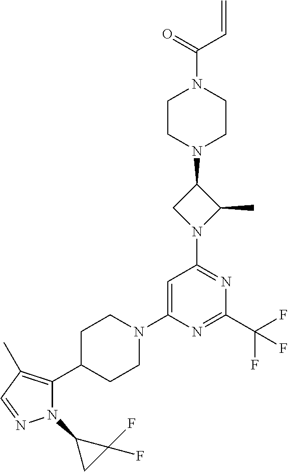 [SMILES: C=CC(=O)N1CCN([C@@H]2CN(c3cc(N4CCC(c5c(C)cnn5[C@@H]5CC5(F)F)CC4)nc(C(F)(F)F)n3)[C@@H]2C)CC1] | 1-(4-((2R,3R)-1-(6- (4-(1-((1R)-2,2- difluorocyclo- propyl-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-234 | 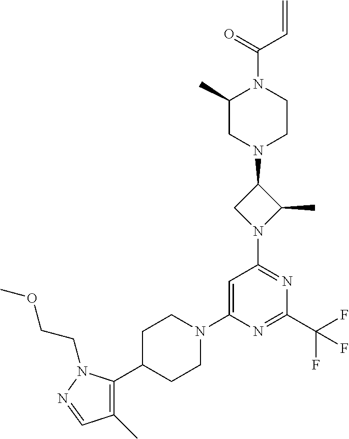 [SMILES: C=CC(=O)N1CCN([C@@H]2CN(c3cc(N4CCC(c5c(C)cnn5CCOC)CC4)nc(C(F)(F)F)n3)[C@@H]2C)C[C@H]1C] | 1-((2R)-4- ((2R,3R)-1-(6-(4- (1-(2- methoxyethyl)-4- methyl-1H- pyrazol-5-yl)-1- piperidinyl)-2- (trifluoromethyl)- 4-pyrimidinyl)-2- methyl-3- azetidinyl)-2- methyl-1- piperazinyl)-2- propen-1-one | 2-238 | 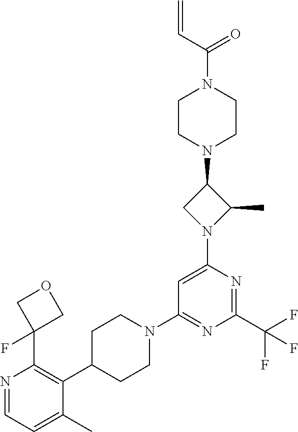 [SMILES: C=CC(=O)N1CCN([C@@H]2CN(c3cc(N4CCC(c5c(C)ccnc5C5(F)COC5)CC4)nc(C(F)(F)F)n3)[C@@H]2C)CC1] | 1-(4-((2R,3R)-1-(6- (4-(2-(3-fluoro-3- oxetanyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-235 | 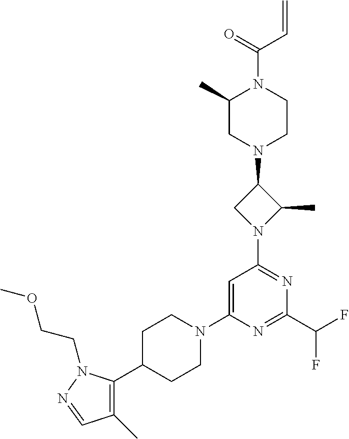 [SMILES: C=CC(=O)N1CCN([C@@H]2CN(c3cc(N4CCC(c5c(C)cnn5CCOC)CC4)nc(C(F)F)n3)[C@@H]2C)C[C@H]1C] | 1-((2R)-4- ((2R,3R)-1-(2- (difluoromethyl)-6- (4-(1-(2- methoxyethyl)-4- methyl-1H- pyrazol-5-yl)-1- piperidinyl)-4 pyrimidinyl)-2- methyl-3- azetidinyl)-2- methyl-1- piperazinyl)-2- propen-1-one | 2-239 | 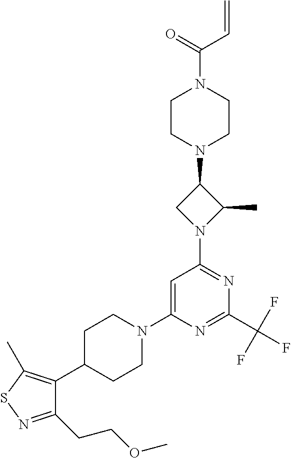 [SMILES: C=CC(=O)N1CCN([C@@H]2CN(c3cc(N4CCC(c5c(CCOC)nsc5C)CC4)nc(C(F)(F)F)n3)[C@@H]2C)CC1] | 1-(4-((2R,3R)-1-(6- (4-(3-(2- methoxyethyl)-5- methyl-1,2-thiazol- 4-yl)-1- piperidiny])-2- (trifluoromethyl)- 4-pyrimidinyl)-2- methyl-3= azetidinyl)-1- piperazinyl)-2- propen-1-one |
| 2-236 | 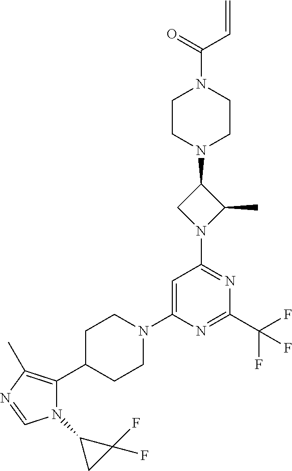 [SMILES: C=CC(=O)N1CCN([C@@H]2CN(c3cc(N4CCC(c5c(C)ncn5[C@H]5CC5(F)F)CC4)nc(C(F)(F)F)n3)[C@@H]2C)CC1] | 1-(4-((2R,3R)-1-(6- (4-(1-((IS)-2,2- difluorocyclo- propyl)-4- moetby-1-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-240 | 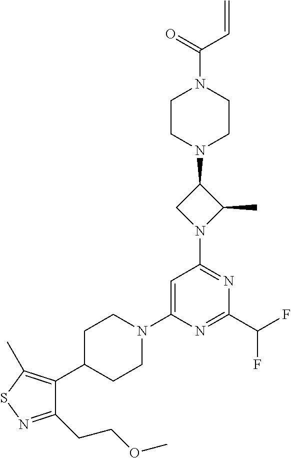 [SMILES: C=CC(=O)N1CCN([C@@H]2CN(c3cc(N4CCC(c5c(CCOC)nsc5C)CC4)nc(C(F)F)n3)[C@@H]2C)CC1] | 1-(4-((2R,3R)-1-(2- (difluoromethy])-6- (4-(3-(2- methoxyethyl)-5- methyl-1,2-thiazol- 4-yl)-1- piperidiny])-4- pyrimidinyl)-2- methyl-3- azetidinyl)-1- piperazinyl)-2- propen-1-one |
| 2-241 | 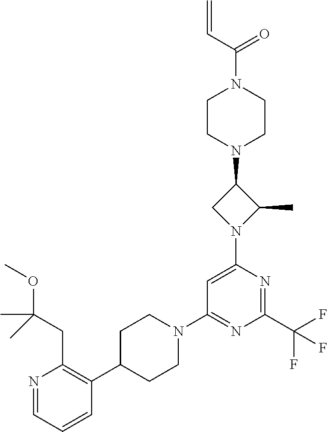 [SMILES: C=CC(=O)N1CCN([C@@H]2CN(c3cc(N4CCC(c5cccnc5CC(C)(C)OC)CC4)nc(C(F)(F)F)n3)[C@@H]2C)CC1] | 1-(4-((2R,3R)-1-(6- (4-(2-(2-methoxy- 2-methylpropyl)-3- pyridinyl)-1- piperidiny])-2- (trifluoromethyl)- 4-pyrimidinyl)-2- methyl-3- azetidinyl)-1- piperazinyl)-2- propen-1-one | 2-245 | 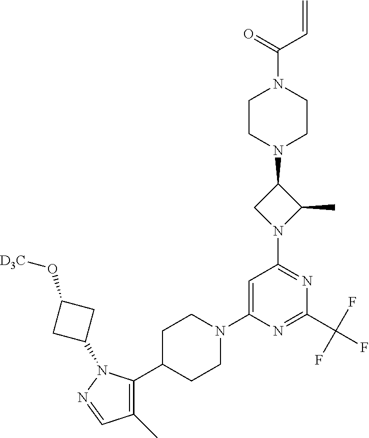 | 1-(4-((2R,3R)-2- methyl-1-(6-(4-(4- methyl-1-(cis-3- ((-2-H_3_)methyl oxy)cyclobutyl)- 1H-pyrazol-5-yl)- 1-piperidinyl)-2- (trifluoromethyl)- 4-pyrimidinyl)-3- azetidinyl)-1- piperazinyl)-2- propen-1-one |
| 2-242 | 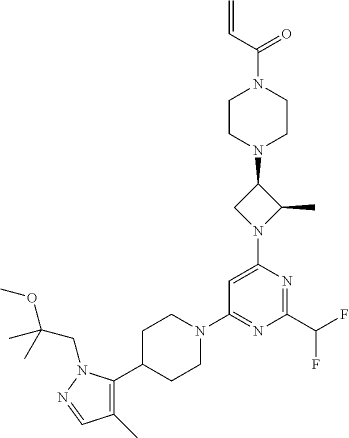 [SMILES: C=CC(=O)N1CCN([C@@H]2CN(c3cc(N4CCC(c5c(C)cnn5CC(C)(C)OC)CC4)nc(C(F)F)n3)[C@@H]2C)CC1] | 1-(4-((2R,3R)-1-(2- (difluoromcthyl)-6- (4-(1-(2-methoxy- 2-methylpropyl)-4- methyl-1H- pyrazol-5-yl)-1- piperidinyl)-4- pyrimidinyl)-2- methyl-3- azetidinyl)-1- piperazinyl)-2- propen-1-one | 2-246 | 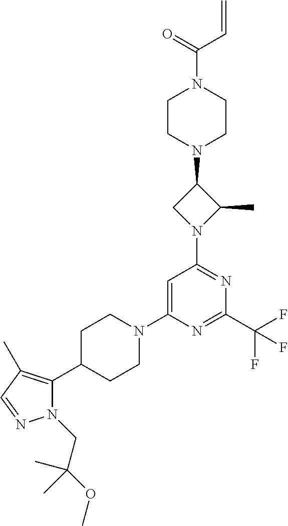 [SMILES: C=CC(=O)N1CCN([C@@H]2CN(c3cc(N4CCC(c5c(C)cnn5CC(C)(C)OC)CC4)nc(C(F)(F)F)n3)[C@@H]2C)CC1] | 1-(4-((2R,3R)-1-(6- (4-(1-(2-methoxy- 2-methylpropyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-243 | 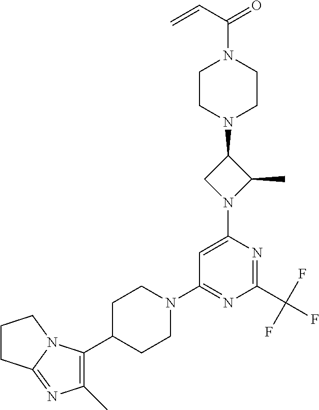 [SMILES: C=CC(=O)N1CCN([C@@H]2CN(c3cc(N4CCC(c5c(C)nc6n5CCC6)CC4)nc(C(F)(F)F)n3)[C@@H]2C)CC1] | 1-(4-((2R.3R)-2- methyl-1-(6-(4-(2- methyl-6,7- dihydro-5H- pyrrolof1,2- a]imidazol-3-yl)-1- piperidinyl)-2- (trifluoromethyl)- 4-pyrimidinyl)-3- azetidinyl)-1- piperazinyl)-2- propen-1-one | 2-247 | 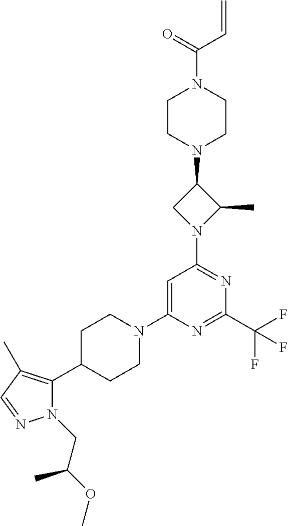 [SMILES: C=CC(=O)N1CCN([C@@H]2CN(c3cc(N4CCC(c5c(C)cnn5C[C@H](C)OC)CC4)nc(C(F)(F)F)n3)[C@@H]2C)CC1] | 1-(4-((2R,3R)-1-(6- (4-(1-((2S)-2- methoxypropyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-244 | 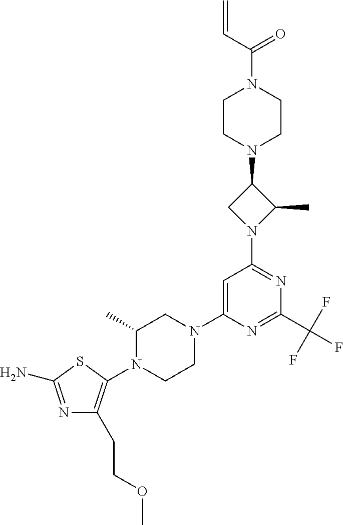 [SMILES: C=CC(=O)N1CCN([C@@H]2CN(c3cc(N4CCN(c5sc(N)nc5CCOC)[C@H](C)C4)nc(C(F)(F)F)n3)[C@@H]2C)CC1] | 1-(4-((2R,3R)-1-(6- ((3R)-4-(2-amino- 4-(2- methoxyethyl)-1,3- thiazol-5-yl)-3- nicthyl-1- piperazinyl)-2- (trifluoromethyl)- 4-pyrimidinyl)-2- methyl-3- azetidinyl)-1- piperazinyl)-2- propen-1-one | 2-248 | 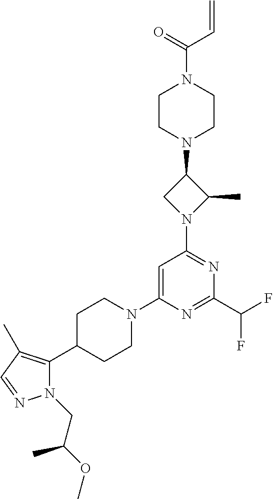 [SMILES: C=CC(=O)N1CCN([C@@H]2CN(c3cc(N4CCC(c5c(C)cnn5C[C@H](C)OC)CC4)nc(C(F)F)n3)[C@@H]2C)CC1] | 1-(4-((2R,3R)-1-(2- (difluoromethy])-6- (4-(1-((2S)-2- methoxypropyl)-4- methyl-1H- pyrazol-5-yl)-1- piperidiny])-4- pyrimidinyl)-2- methyl-3- azetidinyl)-1- piperazinyl)-2- propen-1-one |
| 2-249 | 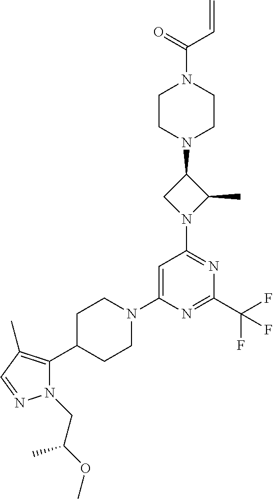 [SMILES: C=CC(=O)N1CCN([C@@H]2CN(c3cc(N4CCC(c5c(C)cnn5C[C@@H](C)OC)CC4)nc(C(F)(F)F)n3)[C@@H]2C)CC1] | 1-(4-((2R,3R)-1-(6- (4-(1-((2R)-2- methoxypropyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-253 | 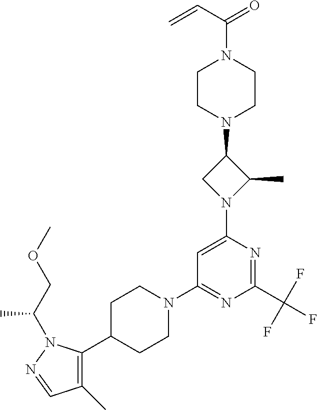 [SMILES: C=CC(=O)N1CCN([C@@H]2CN(c3cc(N4CCC(c5c(C)cnn5[C@H](C)COC)CC4)nc(C(F)(F)F)n3)[C@@H]2C)CC1] | 1-(4-((2R,3R)-1-(6- (4-(1-((2R)-1- methoxy-2- propanyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-250 | 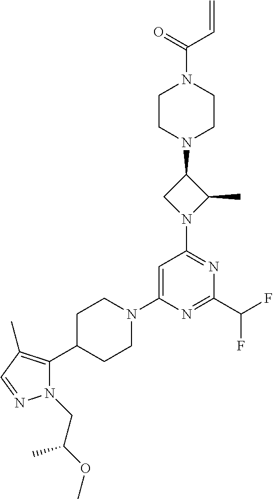 [SMILES: C=CC(=O)N1CCN([C@@H]2CN(c3cc(N4CCC(c5c(C)cnn5C[C@@H](C)OC)CC4)nc(C(F)F)n3)[C@@H]2C)CC1] | 1-(4-((2R,3R)-1-(2- (difluoromethyl)-6- (4-(1-((2R)-2- methoxypropyl)-4- methyl-1H- pyrazol-5-yl)-1- piperidinyl)-4- pyrimidinyl)-2- methyl-3- azetidinyl)-1- piperazinyl)-2- propen-1-one | 2-254 | 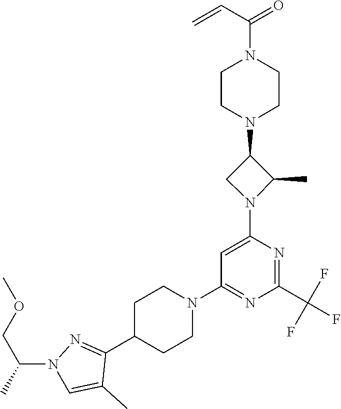 [SMILES: C=CC(=O)N1CCN([C@@H]2CN(c3cc(N4CCC(c5nn([C@H](C)COC)cc5C)CC4)nc(C(F)(F)F)n3)[C@@H]2C)CC1] | 1-(4-((2R,3R)-1-(6- (4-(1-((2R)-1- methoxy-2- propanyl)-4- methyl-1H- pyrazol-3-yl)-1- piperidinyl)-2- (trifluoromethyl)- 4-pyrimidinyl)-2- methyl-3- azetidinyl)-]- piperazinyl)-2- propen-1-one |
| 2-251 | 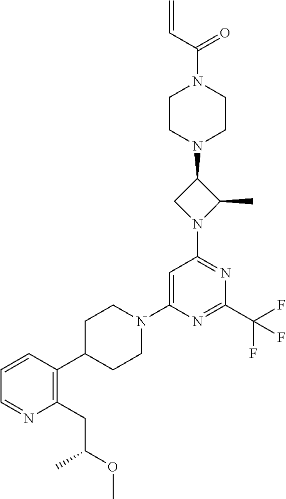 [SMILES: C=CC(=O)N1CCN([C@@H]2CN(c3cc(N4CCC(c5cccnc5C[C@@H](C)OC)CC4)nc(C(F)(F)F)n3)[C@@H]2C)CC1] | 1-(4-((2R,3R)-1-(6- (4-(2-((2R)-2- methoxypropyl)-3- pyridinyl)-1- piperidinyl)-2- (trifluoromethyl)- 4-pyrimidinyl)-2- methyl-3- azetidinyl)-1- piperazinyl)-2- propen-1-one | 2-255 | 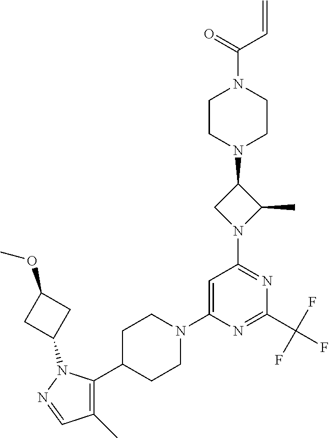 [SMILES: C=CC(=O)N1CCN([C@@H]2CN(c3cc(N4CCC(c5c(C)cnn5[C@H]5C[C@H](OC)C5)CC4)nc(C(F)(F)F)n3)[C@@H]2C)CC1] | 1-(4-((2R,3R)-1-(6- (4-(1-(trans-3- methoxycyclo- butyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-252 | 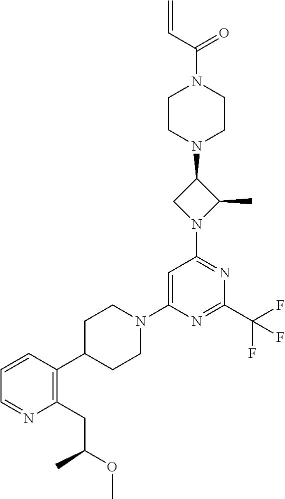 [SMILES: C=CC(=O)N1CCN([C@@H]2CN(c3cc(N4CCC(c5cccnc5C[C@H](C)OC)CC4)nc(C(F)(F)F)n3)[C@@H]2C)CC1] | 1-(4-((2R,3R)-1-(6- (4-(2-((2S)-2- methoxypropyl)-3- pyridinyl)-1- piperidinyl)-2- (trifluoromethyl)- 4-pyrimidinyl)-2- methyl-3- azetidinyl)-1- piperazinyl)-2- propen-1-one | 2-256 | 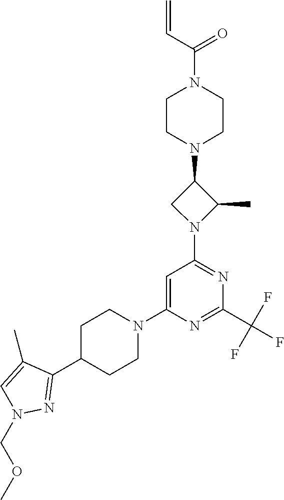 [SMILES: C=CC(=O)N1CCN([C@@H]2CN(c3cc(N4CCC(c5nn(COC)cc5C)CC4)nc(C(F)(F)F)n3)[C@@H]2C)CC1] | 1-(4-((2R,3R)-1-(6- (4-11- (methoxymethyl)- 4-methyl-1H- pyrazol-3-yl)-1- piperidinyl)-2- (trifluoromethyl)- 4-pyrimidinyl)-2- methyl-3- azetidinyl)-1- piperazinyl)-2- propen-1-one |
| 2-257 | 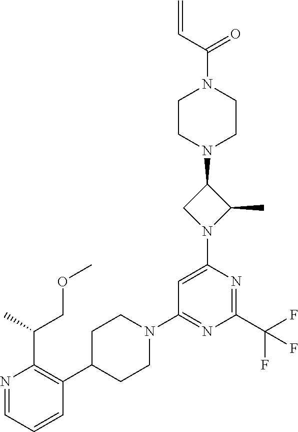 [SMILES: C=CC(=O)N1CCN([C@@H]2CN(c3cc(N4CCC(c5cccnc5[C@H](C)COC)CC4)nc(C(F)(F)F)n3)[C@@H]2C)CC1] | 1-(4-((2R,3R)-1-(6- (4-(2-((2S)-1- methoxy-2- propanyl)-3- pyridinyl)-1- piperidinyl)-2- (trifluoromethyl)- 4-pyrimidiny])-2- methyl-3- azetidinyl)-1- piperazinyl)-2- propen-1-one | 2-261 | 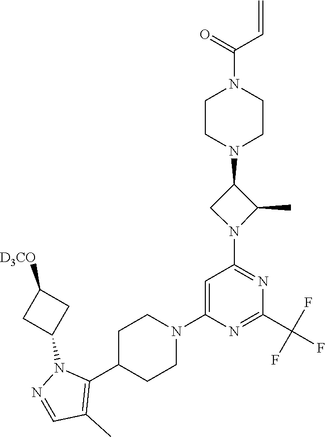 | 1-(4-((2R,3R)-1-(6- (4-(1-((1r,3R)-3- (methoxy- d3)cyclobutyl)-4- methyl-1H- pyrazol-5- yl)piperidin-1-yl)- 2-(trifluoromethyl) pyrimidin-4-yl)-2- methylazetidin-3- yl)piperazin-1- yl)prop-2-en-1-one |
| 2-258 | 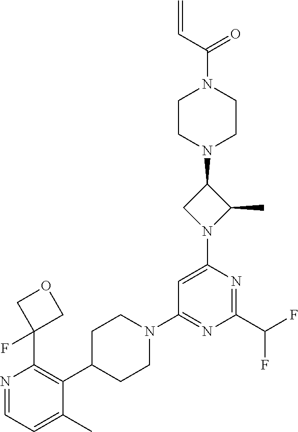 [SMILES: C=CC(=O)N1CCN([C@@H]2CN(c3cc(N4CCC(c5c(C)ccnc5C5(F)COC5)CC4)nc(C(F)F)n3)[C@@H]2C)CC1] | 1-(4-((2R,3R)-1-(2- (difluoromcthyl)-6- (4-(2-(3-fluoro-3- oxetanyl)-4- methyl-3- pytidinyl)-1- piperidinyl)-4- pyrimidinyl)-2- methyl-3- azetidinyl)-1- piperazinyl)-2- propen-1-one | 2-262 | 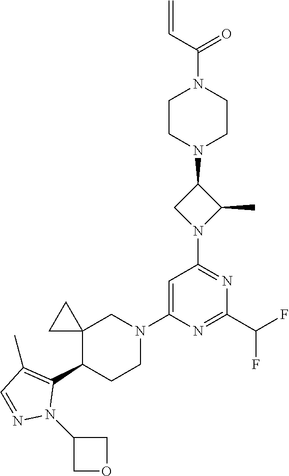 [SMILES: C=CC(=O)N1CCN([C@@H]2CN(c3cc(N4CC[C@@H](c5c(C)cnn5C5COC5)C5(CC5)C4)nc(C(F)F)n3)[C@@H]2C)CC1] | 1-(4-((2R,3R)-1-(2- (difluoromethyl)-6- ((8R)-8-(4-methy]- 1-(3-oxetany])-1H- pyrazol-5-yl)-5- azaspiro[2.5] octan-5-yl)-4- pyrimidinyl)-2- methyl-3- azetidinyl)-1- piperazinyl)-2- propen-1-one |
| 2-259 | 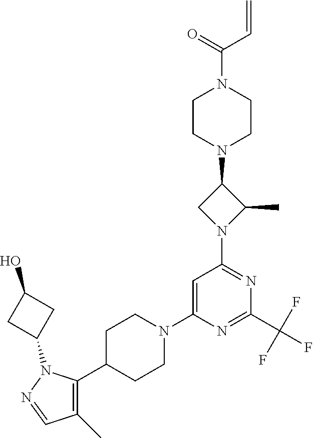 [SMILES: C=CC(=O)N1CCN([C@@H]2CN(c3cc(N4CCC(c5c(C)cnn5[C@H]5C[C@H](O)C5)CC4)nc(C(F)(F)F)n3)[C@@H]2C)CC1] | 1-(4-((2R,3R)-1-(6- (4-(1-(trans-3- hydroxycyclo- butyl)-4-methyl- 1H-pyrazol-5-yl)- 1-piperidinyl)-2- (trifluoromethyl)- 4-pyrimidinyl)-2- methyl-3- azetidinyl)-1- piperazinyl)-2- propen-1-one | 2-263 | 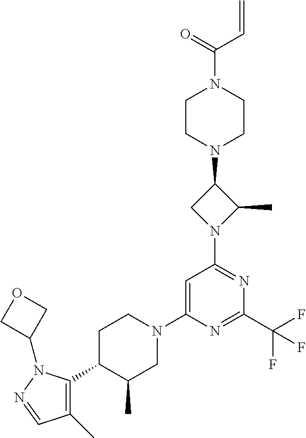 [SMILES: C=CC(=O)N1CCN([C@@H]2CN(c3cc(N4CC[C@@H](c5c(C)cnn5C5COC5)[C@H](C)C4)nc(C(F)(F)F)n3)[C@@H]2C)CC1] | 1-(4-((2R.3R)-2- methyl-1-(6- ((3S,4R)-3-methyl- 4-(4-methyl-1-(3- oxetanyl)-1H- pyrazol-5-yl)-1- piperidinyl)-2- (trifluoromethyl)- 4-pyrimidinyl)-3- azetidinyl)-1- piperazinyl)-2- propen-1-one |
| 2-260 | 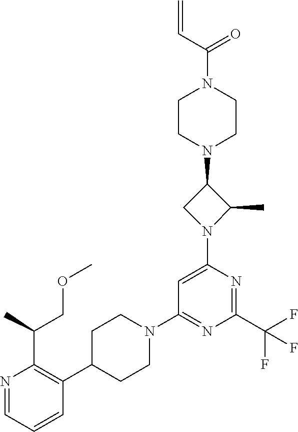 [SMILES: C=CC(=O)N1CCN([C@@H]2CN(c3cc(N4CCC(c5cccnc5[C@@H](C)COC)CC4)nc(C(F)(F)F)n3)[C@@H]2C)CC1] | 1-(4-((2R,3R)-1-(6- (4-(2-((2R)-1- methoxy-2- propanyl)-3- pyridinyl)-1- piperidinyl)-2- (trifluoromethyl)- 4-pyrimidinyl)-2- methyl-3- azetidinyl)-1- piperazinyl)-2- propen-1-one | 2-264 | 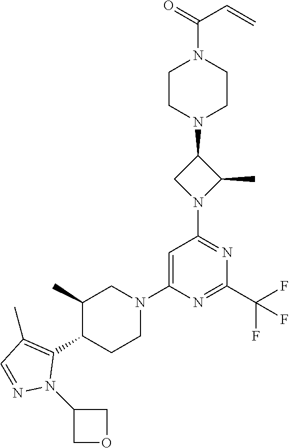 [SMILES: C=CC(=O)N1CCN([C@@H]2CN(c3cc(N4CC[C@H](c5c(C)cnn5C5COC5)[C@@H](C)C4)nc(C(F)(F)F)n3)[C@@H]2C)CC1] | 1-(4-((2R,3R)-2- methyl-1-(6- ((3R,4S)-3-methyl- 4-(4-methyl-1-(3- oxetanyl)-1H- pyrazol-5-yl)-1- piperidiny])-2- (trifluoromethyl)- 4-pyrimidinyl)-3- azetidinyl)-1- piperazinyl)-2- propen-1-one |
| 2-265 | 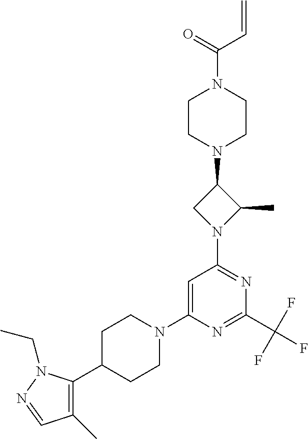 [SMILES: C=CC(=O)N1CCN([C@@H]2CN(c3cc(N4CCC(c5c(C)cnn5CC)CC4)nc(C(F)(F)F)n3)[C@@H]2C)CC1] | 1-(4-((2R,3R)-1-(6- (4-(1-ethyl-4- methyl-1H- pyrazol-5-yl)-1- piperidiny])-2- (trifluoromethyl)- 4-pyrimidinyl)-2- methyl-3- azetidinyl)-1- piperazinyl)-2- propen-1-one | 2-269 | 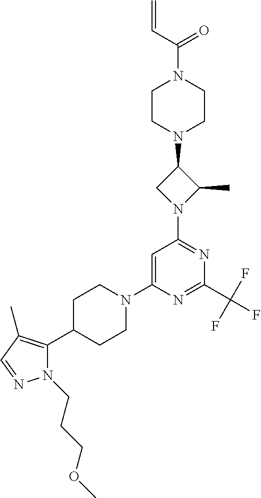 [SMILES: C=CC(=O)N1CCN([C@@H]2CN(c3cc(N4CCC(c5c(C)cnn5CCCOC)CC4)nc(C(F)(F)F)n3)[C@@H]2C)CC1] | 1-(4-((2R,3R)-1-(6- (4-(1-(3- methoxypropyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-266 | 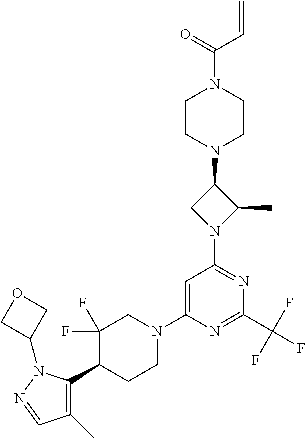 [SMILES: C=CC(=O)N1CCN([C@@H]2CN(c3cc(N4CC[C@@H](c5c(C)cnn5C5COC5)C(F)(F)C4)nc(C(F)(F)F)n3)[C@@H]2C)CC1] | 1-(4-((2R,3R)-1-(6- ((4S)-3,3-difluoro- 4-(4-methy]-1-(3- oxetanyl)-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-270 | 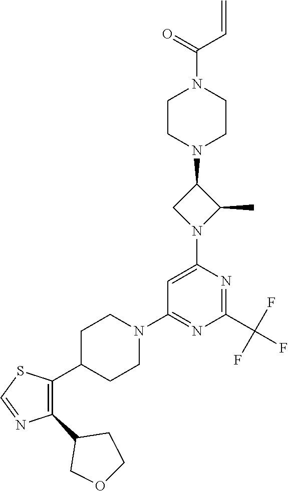 | 1-(4-((2R,3R)-2- methyl-1-(6-(4-(4- ((3R)-tetrahydro-3- furanyl)-1,3- thiazol-5-yl)-1- piperidinyl)-2- (trifluoromethyl)- 4-pyrimidinyl)-3- azetidinyl)-1- piperaziny])-2- propen-1-one |
| 2-267 | 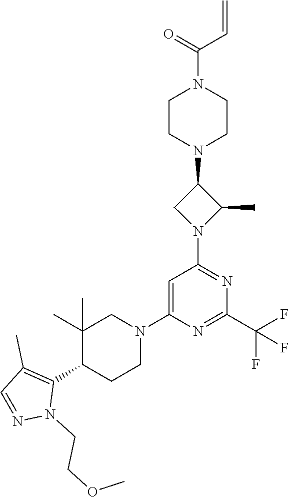 [SMILES: C=CC(=O)N1CCN([C@@H]2CN(c3cc(N4CC[C@H](c5c(C)cnn5CCOC)C(C)(C)C4)nc(C(F)(F)F)n3)[C@@H]2C)CC1] | 1-(4-((2R,3R)-1-(6- ((4S)-4-(1-(2- methoxyethyl)-4- methyl-1H- pyrazol-5-yl)-3,3- dimethyl-1- piperidiny])-2- (trifluoromethyl)- 4-pyrimidinyl)-2- methyl-3- azetidinyl)-1- piperazinyl)-2- propen-1-one | 2-271 | 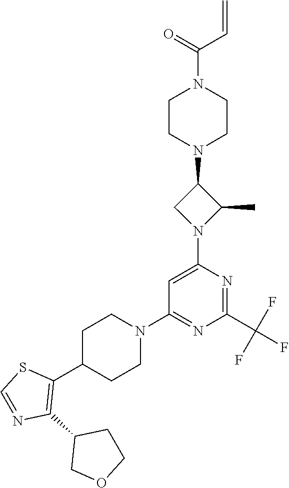 [SMILES: C=CC(=O)N1CCN([C@@H]2CN(c3cc(N4CCC(c5scnc5[C@@H]5CCOC5)CC4)nc(C(F)(F)F)n3)[C@@H]2C)CC1] | 1-(4-((2R,3R)-2- methyl-1-(6-(4-(4- ((3S)-tetrahydro-3- furanyl)-1,3- thiazol-5-yl)-1- piperidiny])-2- (trifluoromethyl)- 4-pyrimidinyl)-3- azetidinyl)-1- piperazinyl)-2- propen-1-one |
| 2-268 | 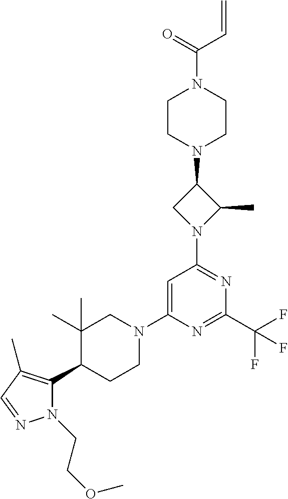 [SMILES: C=CC(=O)N1CCN([C@@H]2CN(c3cc(N4CC[C@@H](c5c(C)cnn5CCOC)C(C)(C)C4)nc(C(F)(F)F)n3)[C@@H]2C)CC1] | 1-(4-((2R,3R)-1-(6- ((4R)-4-(1-(2- methoxyethyl)-4- methyl-1H- pyrazol-5-yl)-3,3- dimethyl-1- piperidinyl)-2- (trifluoromethyl)- 4-pyrimidinyl)-2- methyl-3- azetidinyl)-1- piperazinyl)-2- propen-1-one | 2-272 | 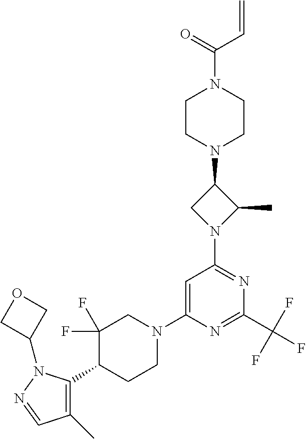 [SMILES: C=CC(=O)N1CCN([C@@H]2CN(c3cc(N4CC[C@H](c5c(C)cnn5C5COC5)C(F)(F)C4)nc(C(F)(F)F)n3)[C@@H]2C)CC1] | 1-(4-((2R,3R)-1-(6- ((4R)-3,3-difluoro- 4-(4-methyl-1-(3- oxetanyl)-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-273 | 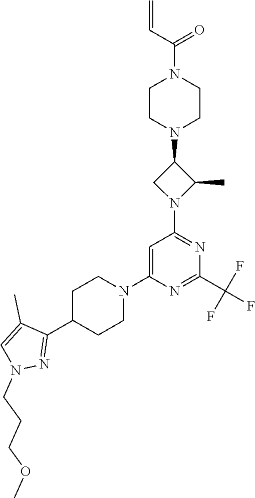 [SMILES: C=CC(=O)N1CCN([C@@H]2CN(c3cc(N4CCC(c5nn(CCCOC)cc5C)CC4)nc(C(F)(F)F)n3)[C@@H]2C)CC1] | 1-(4-((2R,3R)-1-(6- (4-(1-(3- methoxypropyl)-4- methyl-1H- pyrazol-3-yl)-1- piperidinyl)-2- (trifluoromethyl)- 4-pyrimidinyl)-2- methyl-3- azetidinyl)-1- piperazinyl)-2- propen-1-one | 2-277 | 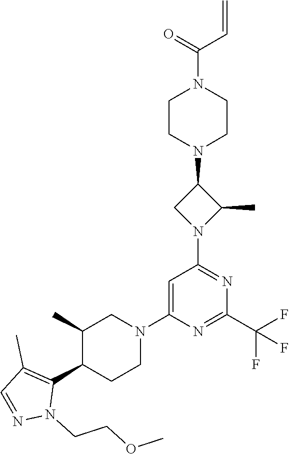 [SMILES: C=CC(=O)N1CCN([C@@H]2CN(c3cc(N4CC[C@@H](c5c(C)cnn5CCOC)[C@@H](C)C4)nc(C(F)(F)F)n3)[C@@H]2C)CC1] | 1-(4-((2R,3R)-1-(6- ((3R,4R)-4-(1-(2- metboxyethyl)-4- methyl-1H- pyrazol-5-yl)-3- methyl-1- piperidinyl)-2- (trifluoromethyl)- 4-pyrimidinyl)-2- methyl-3- azetidinyl)-1- piperazinyl)-2- propen-1-one |
| 2-274 | 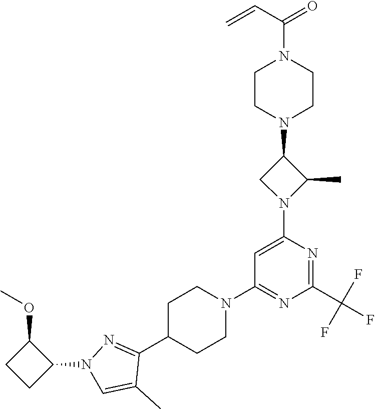 [SMILES: C=CC(=O)N1CCN([C@@H]2CN(c3cc(N4CCC(c5nn([C@@H]6CC[C@H]6OC)cc5C)CC4)nc(C(F)(F)F)n3)[C@@H]2C)CC1] | 1-(4-((2R,3R)-1-(6- (4-(1-((IR,2R)-2- methoxycyclo- butyl)-4-methyl- 1H-pyrazol-3-yl)- 1-piperidiny])-2- (trifluoromethyl)- 4-pyrimidinyl)-2- methyl-3- azetidinyl)-1- piperazinyl)-2- propen-1-one | 2-278 | 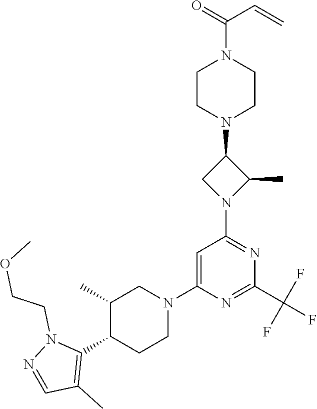 [SMILES: C=CC(=O)N1CCN([C@@H]2CN(c3cc(N4CC[C@H](c5c(C)cnn5CCOC)[C@H](C)C4)nc(C(F)(F)F)n3)[C@@H]2C)CC1] | 1-(4-((2R,3R)-1-(6- ((3S.45)-4-(1-(2- methoxyethyl)-4- methyl-1H- pyrazol-5-yl)-3- methyl-1- piperidinyl)-2- (trifluoromethyl)- 4-pyrintidinyl)-2- methyl-3- azetidinyl)-]- piperazinyl)-2- propen-1-one |
| 2-275 | 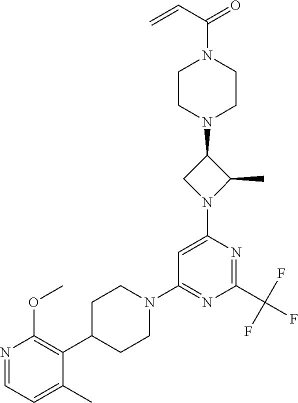 [SMILES: C=CC(=O)N1CCN([C@@H]2CN(c3cc(N4CCC(c5c(C)ccnc5OC)CC4)nc(C(F)(F)F)n3)[C@@H]2C)CC1] | 1-(4-((2R,3R)-1-(6- (4-(2-methoxy-4- methyl-3- pyridinyl)-1- piperidinyl)-2- (trifluoromethyl)- 4-pyrimidinyl)-2- methyl-3- azetidinyl)-1- piperazinyl)-2- propen-1-one | 2-279 | 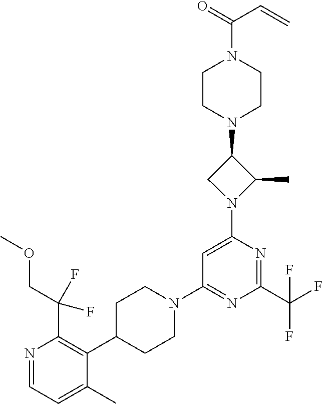 [SMILES: C=CC(=O)N1CCN([C@@H]2CN(c3cc(N4CCC(c5c(C)ccnc5C(F)(F)COC)CC4)nc(C(F)(F)F)n3)[C@@H]2C)CC1] | 1-(4-((2R,3R)-1-(6- (4-(2-(1,1-difluoro- 2-methoxyethyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-276 | 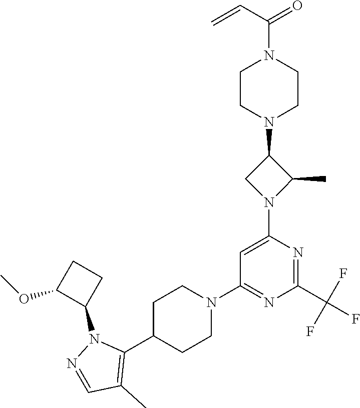 [SMILES: C=CC(=O)N1CCN([C@@H]2CN(c3cc(N4CCC(c5c(C)cnn5[C@@H]5CC[C@H]5OC)CC4)nc(C(F)(F)F)n3)[C@@H]2C)CC1] | 1-(4-((2R,3R)-1-(6- (4-(1-((IR,2R)-2- methoxycyclobutyl )-4-methyl-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-280 | 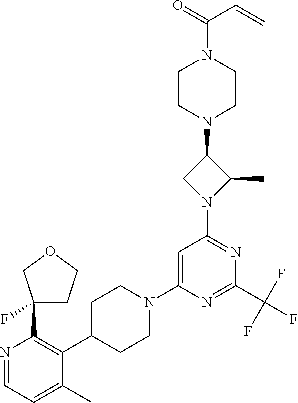 [SMILES: C=CC(=O)N1CCN([C@@H]2CN(c3cc(N4CCC(c5c(C)ccnc5[C@]5(F)CCOC5)CC4)nc(C(F)(F)F)n3)[C@@H]2C)CC1] | 1-(4-((2R,3R)-1-(6- (4-(2-((3R)-3- fluorotetrahydro-3- furanyl)-4-methyl- 3-pyridinyl)-1- piperidinyl)-2- (trifluoromethyl)- 4-pyrimidinyl)-2- methyl-3- azetidinyl)-1- piperazinyl)-2- propen-1-one |
| 2-281 | 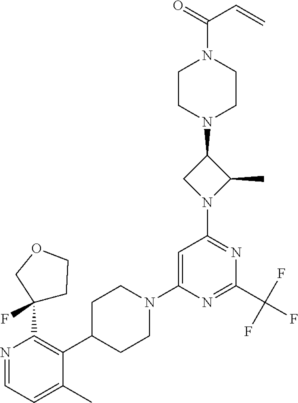 [SMILES: C=CC(=O)N1CCN([C@@H]2CN(c3cc(N4CCC(c5c(C)ccnc5[C@@]5(F)CCOC5)CC4)nc(C(F)(F)F)n3)[C@@H]2C)CC1] | 1-(4-((2R,3R)-1-(6- (4-(2-((38)-3- fluorotetrahydro-3- furanyl)-4-methyl- 3-pyridinyl)-1- piperidinyl)-2- (trifluoromethyl)- 4-pyrimidinyl)-2- methyl-3- azetidinyl)-1- piperazinyl)-2- propen-1-one | 2-285 | 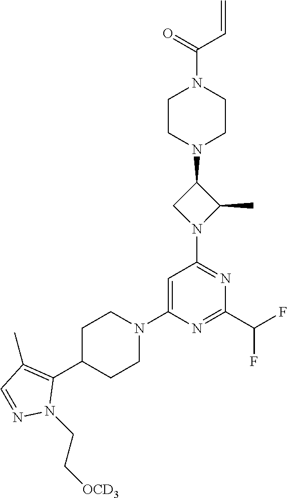 | 1-(4-((2R.3R)-1-(2- (difluoromethyl)-6- (4-(1-(2-(methoxy- d3)ethyl)-4- methyl-1H- pyrazol-5- yl)piperidin-1- yl)pyrimidin-4-yl)- 2-methylazetidin- 3-yl)piperazin-1- yl)prop-2-en-1-one |
| 2-282 | 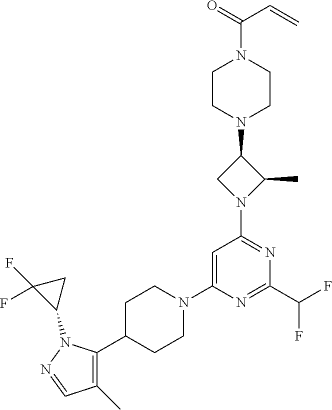 [SMILES: C=CC(=O)N1CCN([C@@H]2CN(c3cc(N4CCC(c5c(C)cnn5[C@H]5CC5(F)F)CC4)nc(C(F)F)n3)[C@@H]2C)CC1] | 1-(4-((2R,3R)-1-(6- (4-(1-((1S)-2,2- difluorocyclo- propyl)-4- methyl-1H- pyrazol-5-yl)-1- piperidinyl)-2- (difluoromethyl)-4- pyrimidinyl)-2- methyl-3- azetidinyl)-1- piperazinyl)-2- propen-1-one | 2-286 | 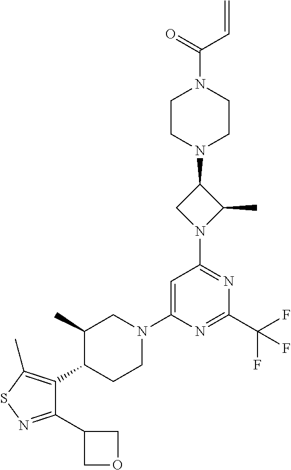 [SMILES: C=CC(=O)N1CCN([C@@H]2CN(c3cc(N4CC[C@H](c5c(C6COC6)nsc5C)[C@@H](C)C4)nc(C(F)(F)F)n3)[C@@H]2C)CC1] | 1-(4-((2R,3R)-2- methyl-1-(6- ((3R,4S)-3-methyl- 4-(5-methyl-3-(3- oxetanyl)-1,2- thiazol-4-yl)-1- piperidinyl)-2- (trifluoromethyl)- 4-pyrimidinyl)-3- azetidinyl)-1- piperazinyl)-2- propen-1-one |
| 2-283 | 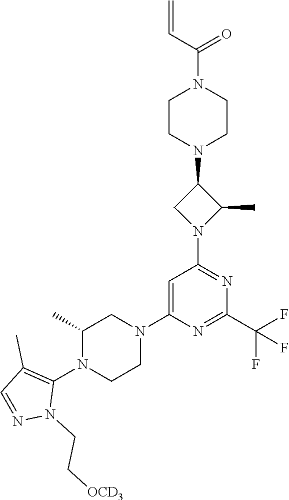 | 1-(4-((2R,3R)-2- methyl-1-(6-((3R)- 3-methyl-4-(4- methyl-1-(2- ((-2-H_3_)methyl oxy)ethyl)-1H- pyrazol-5-yl)-1- piperazinyl)-2- (trifluoromethyl)- 4-pyrimidinyl)-3- azetidinyl)-1- piperazinyl)-2- propen-1-one | 2-287 | 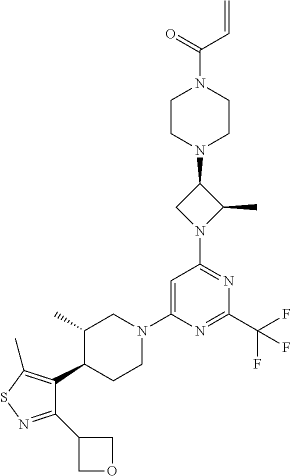 [SMILES: C=CC(=O)N1CCN([C@@H]2CN(c3cc(N4CC[C@@H](c5c(C6COC6)nsc5C)[C@H](C)C4)nc(C(F)(F)F)n3)[C@@H]2C)CC1] | 1-(4-((2R,3R)-2- methyl-1-(6- ((3S,4R)-3-methyl- 4-(5-methyl-3-(3- oxetanyl)-1,2- thiazol-4-yl)-1- piperidinyl)-2- (trifluoromethyl)- 4-pyrimidinyl)-3- azetidinyl)-1- piperazinyl)-2- propen-1-one |
| 2-284 | 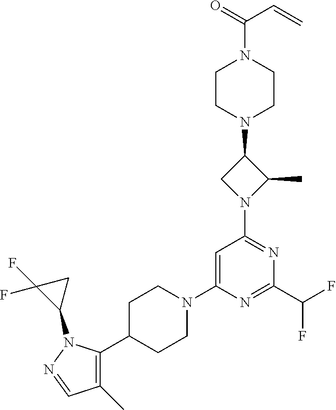 [SMILES: C=CC(=O)N1CCN([C@@H]2CN(c3cc(N4CCC(c5c(C)cnn5[C@@H]5CC5(F)F)CC4)nc(C(F)F)n3)[C@@H]2C)CC1] | 1-(4-((2R,3R)-1-(6- (4-(1-((1R)-2,2- difluorocyclopropy 1)-4-methyl-1H- pyrazol-5-yl)-1- piperidinyl)-2- (difluoromethyl)-4- pyrimidinyl)-2- methyl-3- azetidiny])-1- piperazinyl)-2- propen-1-one | 2-288 | 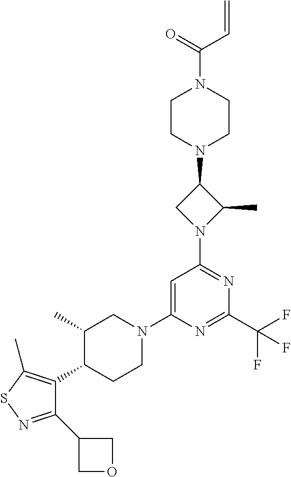 [SMILES: C=CC(=O)N1CCN([C@@H]2CN(c3cc(N4CC[C@H](c5c(C6COC6)nsc5C)[C@H](C)C4)nc(C(F)(F)F)n3)[C@@H]2C)CC1] | 1-(4-((2R,3R)-2- methyl-1-(6- ((3S,4S)-3-methyl- 4-(5-methyl-3-(3- oxetanyl)-1,2- thiazol-4-yl)-1- piperidiny])-2- (trifluoromethyl)- 4-pyrimidinyl)-3- azetidiny])-1- piperazinyl)-2- propen-1-one |
| 2-289 | 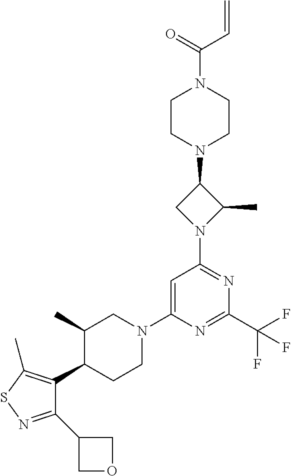 [SMILES: C=CC(=O)N1CCN([C@@H]2CN(c3cc(N4CC[C@@H](c5c(C6COC6)nsc5C)[C@@H](C)C4)nc(C(F)(F)F)n3)[C@@H]2C)CC1] | 1-(4-((2R,3R)-2- methyl-1-(6- ((3R,4R)-3-methyl- 4-(5-methyl-3-(3- oxetanyl)-1,2- thiazol-4-yl)-1- piperidinyl)-2- (trifluoromethyl)- 4-pyrimidinyl)-3- azetidinyl)-1- piperazinyl)-2- propen-1-one | 2-293 | 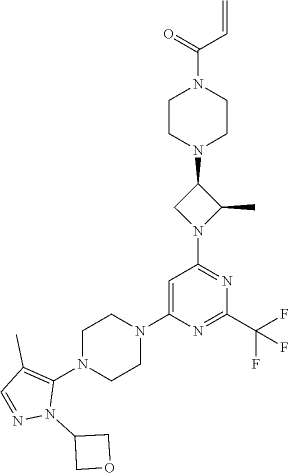 [SMILES: C=CC(=O)N1CCN([C@@H]2CN(c3cc(N4CCN(c5c(C)cnn5C5COC5)CC4)nc(C(F)(F)F)n3)[C@@H]2C)CC1] | 1-(4-((2R,3R)-2- methyl-1-(6-(4-(4- methyl-1-(3- oxetanyl)-1H- pyrazol-5-yl)-1- piperazinyl)-2- (trifluoromethyl)- 4-pyrimidinyl)-3- azetidinyl)-1- piperazinyl)-2- propen-1-one |
| 2-290 | 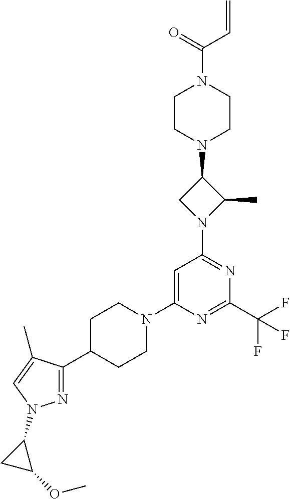 [SMILES: C=CC(=O)N1CCN([C@@H]2CN(c3cc(N4CCC(c5nn([C@H]6C[C@H]6OC)cc5C)CC4)nc(C(F)(F)F)n3)[C@@H]2C)CC1] | 1-(4-((2R,3R)-1-(6- (4-(1-((18,2R)-2- methoxycyclo- propyl)-4- methyl-1H- pyrazol-3-yl)-1- piperidinyl)-2- (trifluoromethyl)- 4-pyrimidinyl)-2- methyl-3- azetidinyl)-1- piperazinyl)-2- propen-1-one | 2-294 | 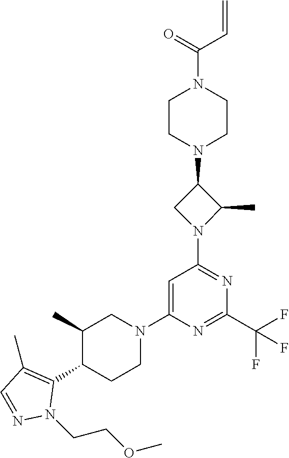 [SMILES: C=CC(=O)N1CCN([C@@H]2CN(c3cc(N4CC[C@H](c5c(C)cnn5CCOC)[C@@H](C)C4)nc(C(F)(F)F)n3)[C@@H]2C)CC1] | 1-(4-((2R,3R)-1-(6- ((3R.4S)-4-(1-(2- methoxyethyl)-4- methyl-1H- pyrazol-5-yl)-3- methyl-1- piperidinyl)-2- (trifluoromethyl)- 4-pyrimidinyl)-2- methyl-3- azetidinyl)-1- piperazinyl)-2- propen-1-one |
| 2-291 | 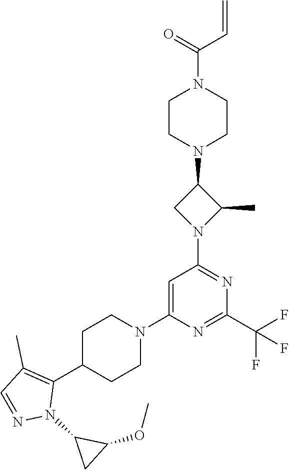 [SMILES: C=CC(=O)N1CCN([C@@H]2CN(c3cc(N4CCC(c5c(C)cnn5[C@H]5C[C@H]5OC)CC4)nc(C(F)(F)F)n3)[C@@H]2C)CC1] | 1-(4-((2R,3R)-1-(6- (4-(1-((1S,2R)-2- methoxycyclo- propyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-295 | 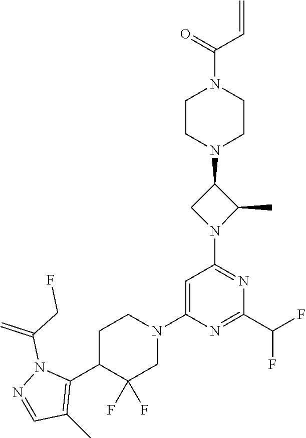 [SMILES: C=CC(=O)N1CCN([C@@H]2CN(c3cc(N4CCC(c5c(C)cnn5C(=C)CF)C(F)(F)C4)nc(C(F)F)n3)[C@@H]2C)CC1] | 1-(4-((2R,3R)-1-(6- (3,3-difluoro-4-(1- (3-fluoro-1-propen- 2-yl)-4-methyl-1H- pyrazol-5-yl)-1- piperidinyl)-2- (difluoromethyl)-4- pyrimidinyl)-2- methyl-3- azetidinyl)-1- piperazinyl)-2- propen-1-one |
| 2-292 | 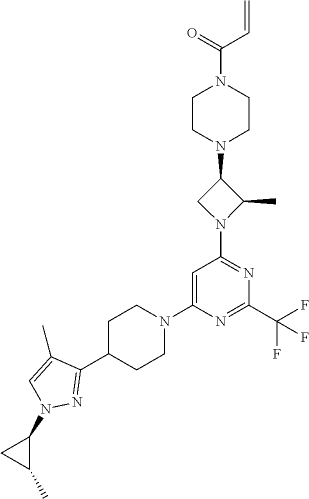 [SMILES: C=CC(=O)N1CCN([C@@H]2CN(c3cc(N4CCC(c5nn([C@@H]6C[C@H]6C)cc5C)CC4)nc(C(F)(F)F)n3)[C@@H]2C)CC1] | 1-(4-((2R,3R)-2- methyl-1-(6-(4-(4- methyl-1-((IR,2R)- 2-methylcyclo- propyl)-[1H- pyrazol-3-yl)- 1-piperidinyl)-2- (trifluoromethyl)- 4-pyrimidinyl)-3- azetidiny])-1- piperazinyl)-2- propen-1-one | 2-296 | 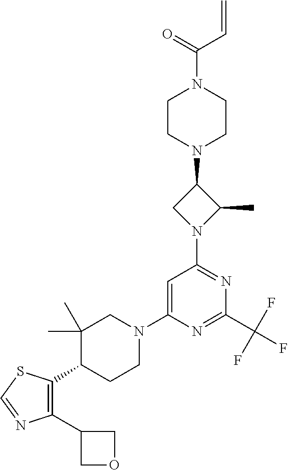 [SMILES: C=CC(=O)N1CCN([C@@H]2CN(c3cc(N4CC[C@H](c5scnc5C5COC5)C(C)(C)C4)nc(C(F)(F)F)n3)[C@@H]2C)CC1] | 1-(4-((2R,3R)-1-(6- ((4S)-3,3-dimethyl- 4-(4-(3-oxetanyl)- 1,3-thiazol-5-yl)-1- pipcridinyl)-2- (trifluoromethyl)- 4-pyrimidinyl)-2- methyl-3- azetidinyl)-1- piperazinyl)-2- propen-t-one |
| 2-297 | 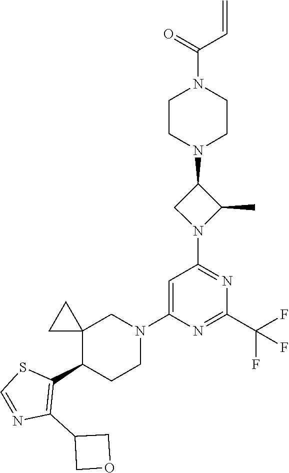 [SMILES: C=CC(=O)N1CCN([C@@H]2CN(c3cc(N4CC[C@@H](c5scnc5C5COC5)C5(CC5)C4)nc(C(F)(F)F)n3)[C@@H]2C)CC1] | 1-(4-((2R,3R)-2- methyl-1-(6-((8R)- 8-(4-(3-oxetanyl)- 1,3-thiazol-5-yl)- 5-azaspiro[2.5] octan-5-yl)-2- (trifluoromcthyl)- 4-pyrimidinyl)-3- azetidinyl)-1- piperazinyl)-2- propen-1-one | 2-301 | 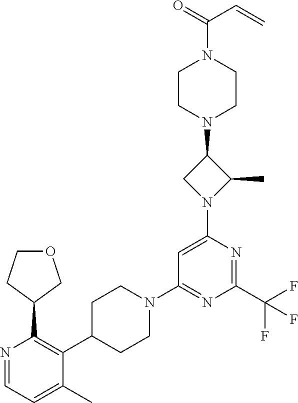 [SMILES: C=CC(=O)N1CCN([C@@H]2CN(c3cc(N4CCC(c5c(C)ccnc5[C@H]5CCOC5)CC4)nc(C(F)(F)F)n3)[C@@H]2C)CC1] | 1-(4-((2R,3R)-2- methyl-1-(6-(4-(4- methyl-2-((3R)- tetrahydro-3- furanyl)-3- pyridinyl)-1- piperidinyl)-2- (trifluoromethyl)- 4-pyrimidinyl)-3- azetidinyl)-1- piperazinyl)-2- propen-1-one |
| 2-298 | 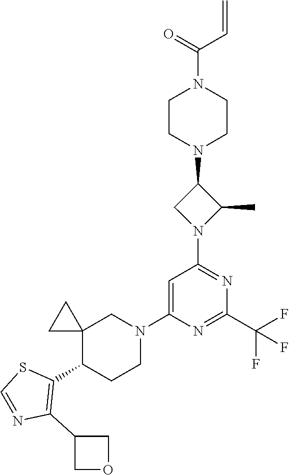 [SMILES: C=CC(=O)N1CCN([C@@H]2CN(c3cc(N4CC[C@H](c5scnc5C5COC5)C5(CC5)C4)nc(C(F)(F)F)n3)[C@@H]2C)CC1] | 1-(4-((2R,3R)-2- methyl-1-(6-((8S)- 8-(4-(3-oxetanyl)- 1,3-thiazol-5-yl)-5- azaspiro[2.5]octan- 5-yl)-2- (trifluoromethyl)- 4-pyrintidinyl)-3- azetidinyl)-1- piperazinyl)-2- propen-1-one | 2-302 | 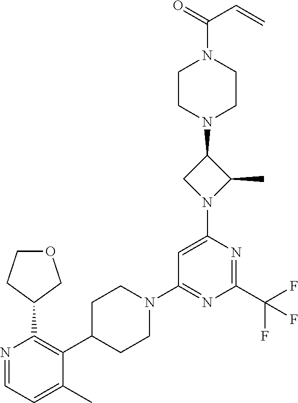 [SMILES: C=CC(=O)N1CCN([C@@H]2CN(c3cc(N4CCC(c5c(C)ccnc5[C@@H]5CCOC5)CC4)nc(C(F)(F)F)n3)[C@@H]2C)CC1] | 1-(4-((2R,3R)-2- methyl-1-(6-(4-(4- methyl-2-((3S)- tetrahydro-3- furanyl)-3- pyridinyl)-1- piperidinyl)-2- (trifluoromethyl)- 4-pyrimidinyl)-3- azetidinyl)-1- piperazinyl)-2- propen-1-one |
| 2-299 | 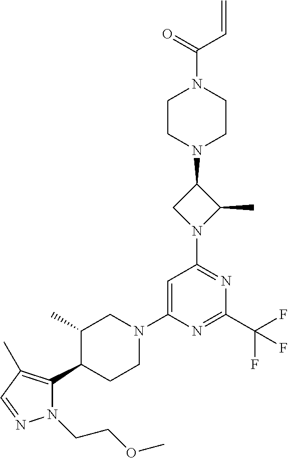 [SMILES: C=CC(=O)N1CCN([C@@H]2CN(c3cc(N4CC[C@@H](c5c(C)cnn5CCOC)[C@H](C)C4)nc(C(F)(F)F)n3)[C@@H]2C)CC1] | 1-(4-((2R,3R)-1-(6- ((3S,4R)-4-(1-(2- methoxyethyl)-4- methyl-1H- pyrazol-5-yl)-3- methyl-1- piperidiny])-2- (trifluoromethyl)- 4-pyrimidinyl)-2- methyl-3- azetidinyl-1- piperazinyl)-2- propen-1-one | 2-303 | 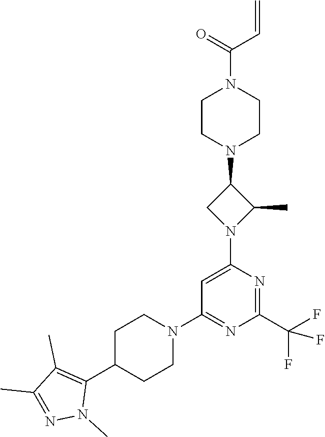 [SMILES: C=CC(=O)N1CCN([C@@H]2CN(c3cc(N4CCC(c5c(C)c(C)nn5C)CC4)nc(C(F)(F)F)n3)[C@@H]2C)CC1] | 1-(4-((2R,3R)-2- methyl-1-(2- (triffnoromethyl)- 6-(4-(1,3,4- trimethyl-1H- pyrazol-5-yl)-1- piperidinyl)-4- pyrimidinyl)-3- azetidinyl)-1- piperazinyl)-2- propen-1-one |
| 2-300 | 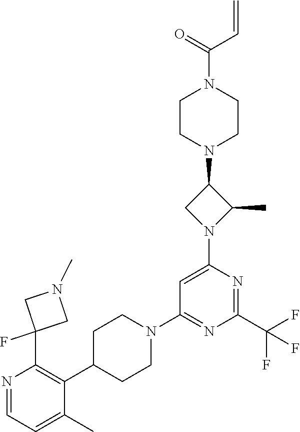 [SMILES: C=CC(=O)N1CCN([C@@H]2CN(c3cc(N4CCC(c5c(C)ccnc5C5(F)CN(C)C5)CC4)nc(C(F)(F)F)n3)[C@@H]2C)CC1] | 1-(4-((2R,3R)-1-(6- (4-(2-(3-fluoro-1- methyl-3- azetidinyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one | 2-304 | 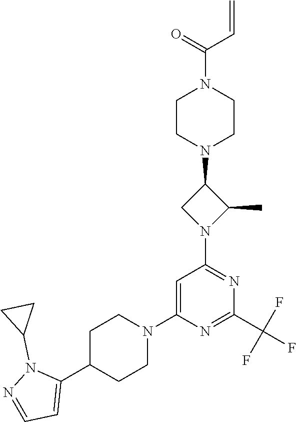 [SMILES: C=CC(=O)N1CCN([C@@H]2CN(c3cc(N4CCC(c5ccnn5C5CC5)CC4)nc(C(F)(F)F)n3)[C@@H]2C)CC1] | 1-(4-((2R,3R)-1-(6- (4-(1-cyclopropyl- 1H-pyrazol-5-yl)- 1-piperidinyl)-2- (trifluoromethyl)- 4-pyrimidinyl)-2- methyl-3- azetidinyl)-1- piperazinyl)-2- propen-1-one |
| 2-305 | 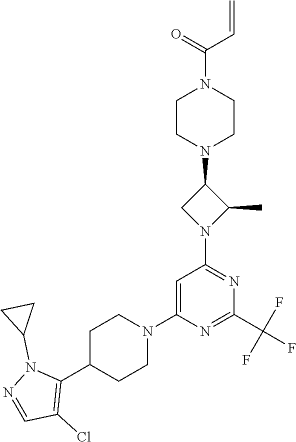 [SMILES: C=CC(=O)N1CCN([C@@H]2CN(c3cc(N4CCC(c5c(Cl)cnn5C5CC5)CC4)nc(C(F)(F)F)n3)[C@@H]2C)CC1] | 1-(4-((2R,3R)-1-(6- (4-(4-chloro-[1- cyclopropyl-1H- pyrazol-5-yl)-1- piperidiny])-2- (trifluoromethyl)- 4-pyrimidinyl)-2- methyl-3- azetidinyl)-1- piperazinyl)-2- propen-1-one | 2-309 | 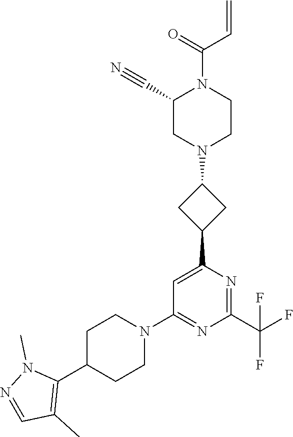 [SMILES: C=CC(=O)N1CCN([C@H]2C[C@H](c3cc(N4CCC(c5c(C)cnn5C)CC4)nc(C(F)(F)F)n3)C2)C[C@@H]1C#N] | (2R)-4-(trans-3-(6- (4-(1,4-dimethyl- 1H-pyrazol-5-yl)- 1-piperidinyl)-2- (trifluoromethyl)- 4.pyrimidinyl) cyclobutyl)-1-(2- propenoyl)-2- piperazine- carbonitrile |
| 2-306 | 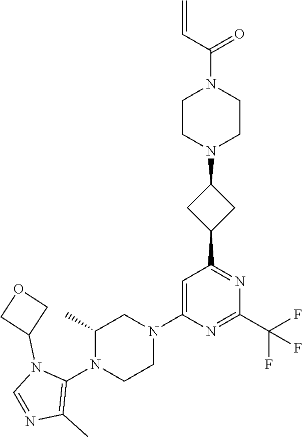 [SMILES: C=CC(=O)N1CCN([C@H]2C[C@@H](c3cc(N4CCN(c5c(C)ncn5C5COC5)[C@H](C)C4)nc(C(F)(F)F)n3)C2)CC1] | 1-(4-(cis-3-(6- ((3R)-3-methyl-4- (4-methyl-1-(3- oxetanyl)-1H- pyrazol-5-yl)-1- piperazinyl)-2- (trifluoromethyl)- 4.pyrimidinyl) cyclobutyl)-1- piperazinyl)-2- propen-1-one | 2-310 | 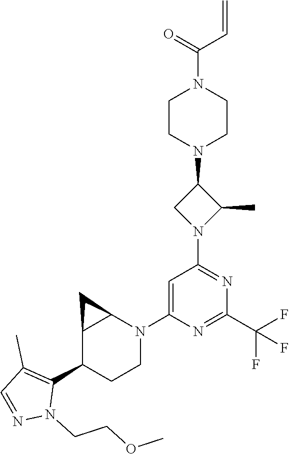 [SMILES: C=CC(=O)N1CCN([C@@H]2CN(c3cc(N4CC[C@@H](c5c(C)cnn5CCOC)[C@H]5C[C@H]54)nc(C(F)(F)F)n3)[C@@H]2C)CC1] | 1-(4-((2R,3R)-1-(6- ((1R,5R,6R)-5-(1- (2-methoxyethyl)- 4-methyl-1H- pyrazol-5-yl)-2- azabicyclo[4.1.0] heptan-2-yl)-2- (trifluoromethyl)- 4-pyrintidinyl)-2- methyl-3- azetidinyl)-1- piperazinyl)-2- propen-1-one |
| 2-307 | 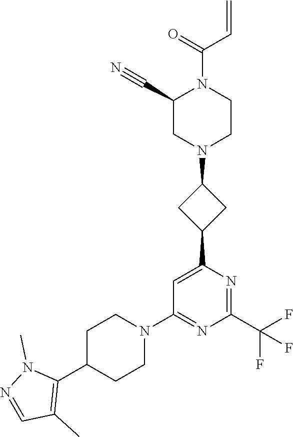 [SMILES: C=CC(=O)N1CCN([C@H]2C[C@@H](c3cc(N4CCC(c5c(C)cnn5C)CC4)nc(C(F)(F)F)n3)C2)C[C@H]1C#N] | (2S-4-(cis-3-(6-(4- (1,4-dimethyl-1H- pyrazol-5-yl)-1- piperidinyl)-2- (trifluoromethyl)- 4-pyrimidinyl) cyclobutyl)-1-(2- propenoyl)-2- piperazine- carbonitrile | 2-311 | 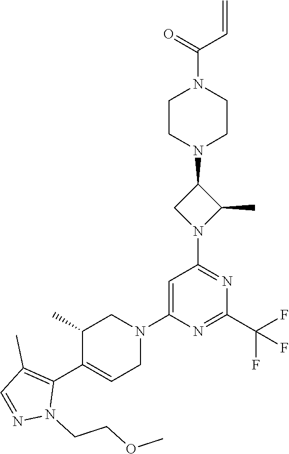 [SMILES: C=CC(=O)N1CCN([C@@H]2CN(c3cc(N4CC=C(c5c(C)cnn5CCOC)[C@H](C)C4)nc(C(F)(F)F)n3)[C@@H]2C)CC1] | 1-(4-((2R,3R)-1-(6- ((3S)-4-(1-(2- methoxyethyl)-4- methyl-1H- pyrazol-5-yl)-3- methyl-3,6- dihydro-1(2H)- pyridinyl)-2- (trifluoromethyl)- 4-pyrimidinyl)-2- methyl-3- azetidinyl)-1- piperazinyl)-2- propen-1-one |
| 2-308 | 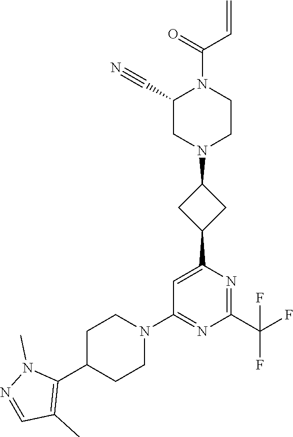 [SMILES: C=CC(=O)N1CCN([C@H]2C[C@@H](c3cc(N4CCC(c5c(C)cnn5C)CC4)nc(C(F)(F)F)n3)C2)C[C@@H]1C#N] | (2R)-4-(cis-3-(6- (4-(1,4-dimethyl- 1H-pyrazol-5-yl)- I-piperidinyl)-2- (trifluoromethyl)- 4-pyrimidinyl) cyclobutyl)-1-(2- propenoyl)-2- piperazine- carbonitrile | 2-312 | 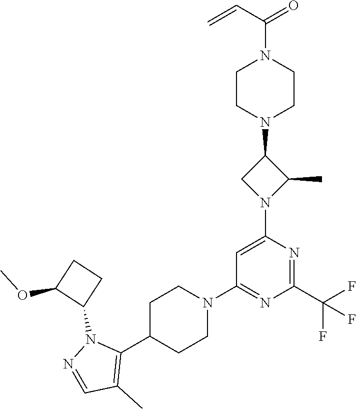 [SMILES: C=CC(=O)N1CCN([C@@H]2CN(c3cc(N4CCC(c5c(C)cnn5[C@H]5CC[C@@H]5OC)CC4)nc(C(F)(F)F)n3)[C@@H]2C)CC1] | 1-(4-((2R,3R)-1-(6- (4-(1-((18,2S)-2- methoxycyclo- butyl)-4-methyl- 1H-pyrazol-5-yl)- 1-piperidinyl)-2- (trifluoromethyl)- 4-pyrimidinyl)-2- methyl-3- azetidinyl)-1- piperazinyl)-2- propen-1-one |
| 2-313 | 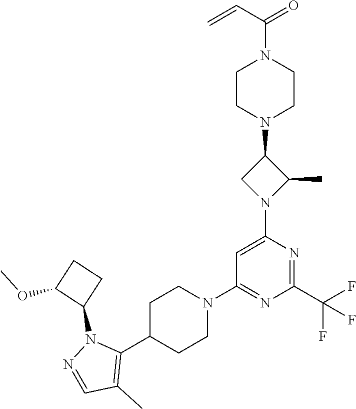 [SMILES: C=CC(=O)N1CCN([C@@H]2CN(c3cc(N4CCC(c5c(C)cnn5[C@@H]5CC[C@H]5OC)CC4)nc(C(F)(F)F)n3)[C@@H]2C)CC1] | 1-(4-((2R,3R)-1-(6- (4-(1-((1R,2R)-2- methoxycyclo- butyl)-4-methyl- 1H-pyrazol-5-yl)-1- piperidinyl)-2- (trifluoromethyl)- 4-pyrimidinyl)-2- methyl-3- azetidinyl)-1- piperazinyl)-2- propen-1-one | 2-317 | 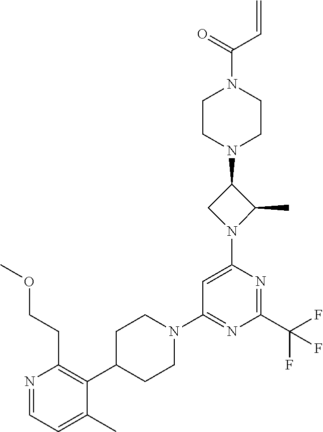 [SMILES: C=CC(=O)N1CCN([C@@H]2CN(c3cc(N4CCC(c5c(C)ccnc5CCOC)CC4)nc(C(F)(F)F)n3)[C@@H]2C)CC1] | 1-(4-((2R,3R)-1-(6- (4-(2-(2- methoxyethyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-314 | 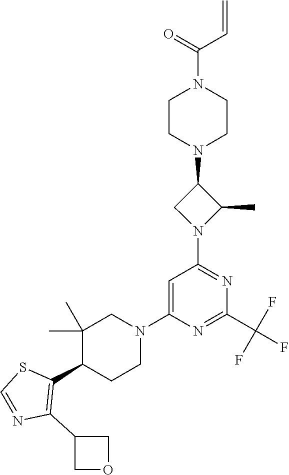 [SMILES: C=CC(=O)N1CCN([C@@H]2CN(c3cc(N4CC[C@@H](c5scnc5C5COC5)C(C)(C)C4)nc(C(F)(F)F)n3)[C@@H]2C)CC1] | 1-(4-((2R,3R)-1-(6- ((4R)-3,3- dimethyl-4-(4-(3- oxetanyl)-1,3- thiazol-5-yl)-1- piperidiny])-2- (trifluoromethyl)- 4-pyrimidinyl)-2- methyl-3- azetidinyl)-1- piperazinyl)-2- propen-1-one | 2-318 | 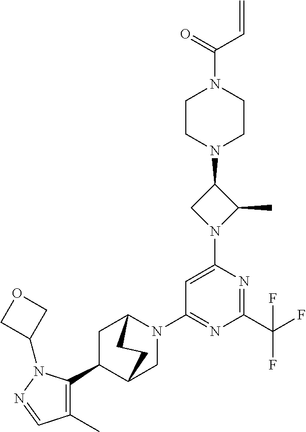 [SMILES: C=CC(=O)N1CCN([C@@H]2CN(c3cc(N4C[C@H]5CC[C@@H]4C[C@@H]5c4c(C)cnn4C4COC4)nc(C(F)(F)F)n3)[C@@H]2C)CC1] | 1-(4-((2R,3R)-2- methyl-1-(6- ((IR,4S,5S)-5-(4- methyl-1-(3- oxetanyl)-1H- pyrazol-5-yl)-2- azabicyclo[2.2.2] octan-2-yl)-2- (trifluoromethyl)- 4-pyrimidinyl)-3- azetidinyl)-1- piperazinyl)-2- propen-1-one |
| 2-315 | 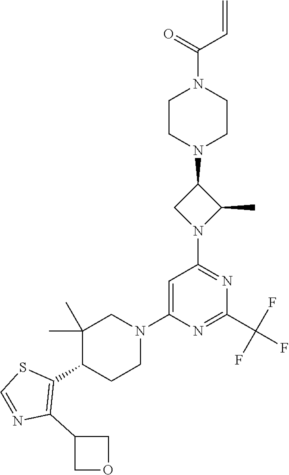 [SMILES: C=CC(=O)N1CCN([C@@H]2CN(c3cc(N4CC[C@H](c5scnc5C5COC5)C(C)(C)C4)nc(C(F)(F)F)n3)[C@@H]2C)CC1] | 1-(4-((2R,3R)-1-(6- ((4S)-3,3-dimethyl- 4-(4-(3-oxetanyl)- 1,3-thiazol-5-yl)-1- piperidinyl)-2- (trifluoromethy])- 4-pyrimidinyl)-2- methyl-3- azetidinyl)-1- piperazinyl)-2- propen-1-one | 2-319 | 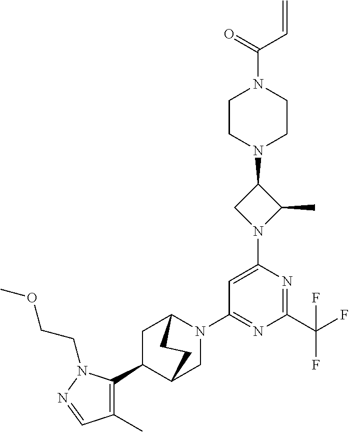 [SMILES: C=CC(=O)N1CCN([C@@H]2CN(c3cc(N4C[C@H]5CC[C@@H]4C[C@@H]5c4c(C)cnn4CCOC)nc(C(F)(F)F)n3)[C@@H]2C)CC1] | 1-(4-((2R,3R)-1-(6- ((1R,4S,5S)-5-(1- (2-methoxyethyl)- 4-methyl-1H- pyrazol-5-yl)-2- azabicyclo[2.2.2] octan-2-yl)-2- (trifluoromethyl)- 4-pyrimidinyl)-2- methyl-3- azetidinyl)-1- piperazinyl)-2- propen-1-one |
| 2-316 | 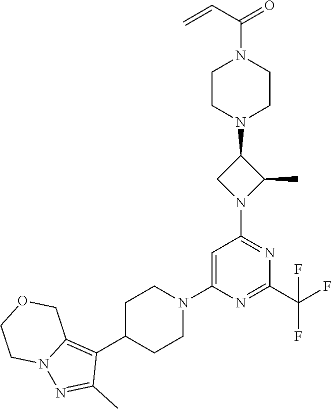 [SMILES: C=CC(=O)N1CCN([C@@H]2CN(c3cc(N4CCC(c5c(C)nn6c5COCC6)CC4)nc(C(F)(F)F)n3)[C@@H]2C)CC1] | 1-(4-((2R,3R)-2- methyl-1-(6-(4-(2- methyl-6,7- dihydro-4H- pyrazolo[5,1- c][1,4]oxazin-3- yl)-1-piperidinyl)- 2- (trifluoromethyl)- 4-pyrimidinyl)-3- azetidinyl)-1- piperazinyl)-2- propen-1-one | 2-320 | 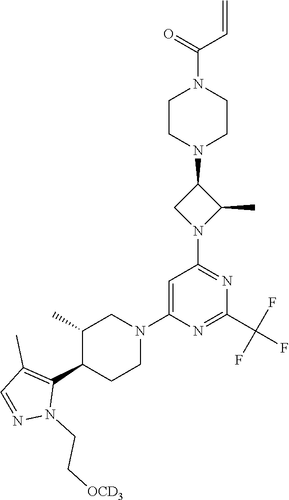 | 1-(4-((2R,3R)-1-(6- ((3S,4R)-4-(1-(2- (methoxy- d3)ethyl)-4- methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one |
| 2-321 | 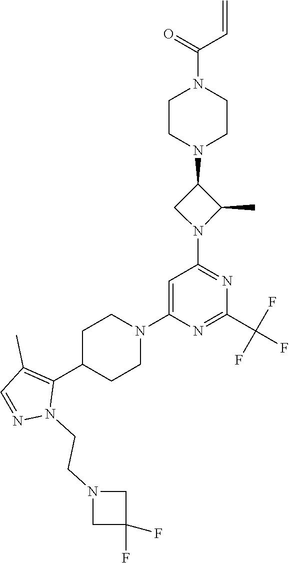 [SMILES: C=CC(=O)N1CCN([C@@H]2CN(c3cc(N4CCC(c5c(C)cnn5CCN5CC(F)(F)C5)CC4)nc(C(F)(F)F)n3)[C@@H]2C)CC1] | 1-(4-((2R,3R)-1-(6- (4-(1-(2-(3,3- difluoro-1- azetidinyl)ethyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-325 | 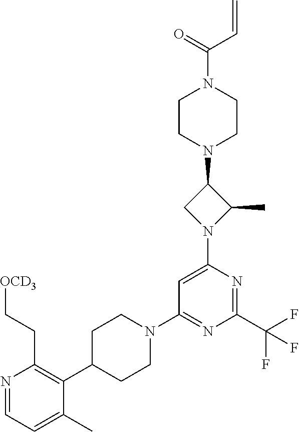 | 1-(4-((2R.3R)-1-(6- (4-(2-(2-(methoxy- d3)ethyl)-4- methylpyridin-3- yl)piperidin-1-yl)- 2. (trifluoromethyl) pyrimidin-4-yl)-2- methylazetidin-3- yl)piperazin-1- yl)prop-2-en-1-one |
| 2-322 | 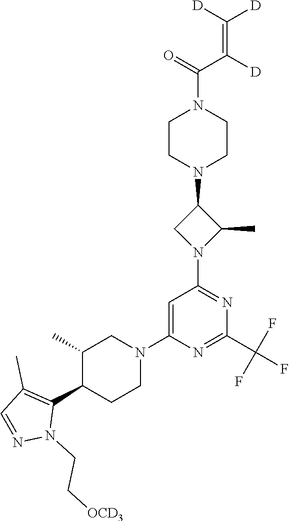 | 1-(4-((2R.3R)-1-(6- ((3S,4R)-4-(1-(2- (methoxy- (3)ethyl)-4- methyl-1H- pyrazol-5-yl)-3. methylpiperidin-1- yl)-2-(trifluoromethyl) pyrimidin-4-yl)-2- methylazetidin-3- yl)piperazin-1- yl)prop-2-en-1- one-2,3,3-d3 | 2-326 | 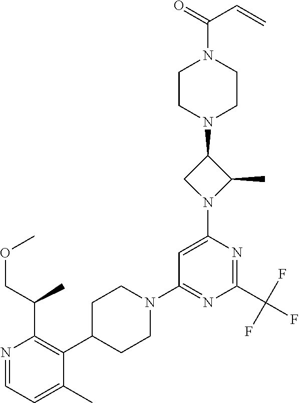 [SMILES: C=CC(=O)N1CCN([C@@H]2CN(c3cc(N4CCC(c5c(C)ccnc5[C@H](C)COC)CC4)nc(C(F)(F)F)n3)[C@@H]2C)CC1] | 1-(4-((2R,3R)-1-(6- (4-(2-((2S)-1- methoxy-2- propanyl)-4- methyl-3- pyridinyl)-1- piperidinyl)-2- (trifluoromethyl)- 4-pyrintidinyl)-2- methyl-3- azetidinyl)-]- piperazinyl)-2- propen-1-one |
| 2-323 | 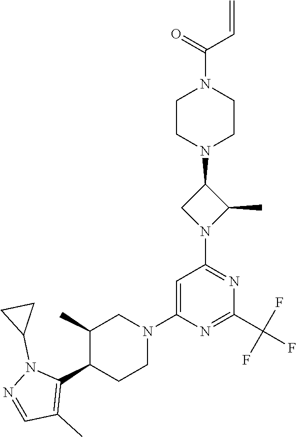 [SMILES: C=CC(=O)N1CCN([C@@H]2CN(c3cc(N4CC[C@@H](c5c(C)cnn5C5CC5)[C@@H](C)C4)nc(C(F)(F)F)n3)[C@@H]2C)CC1] | 1-(4-((2R,3R)-1-(6- ((3R,4R)-4-(1- cyclopropyl-4- methyl-1H- pyrazol-5-yl)-3- methyl-1- piperidiny])-2- (trifluoromethyl)- 4-pyrimidinyl)-2- methyl-3- azetidinyl-1- piperazinyl)-2- propen-1-one | 2-327 | 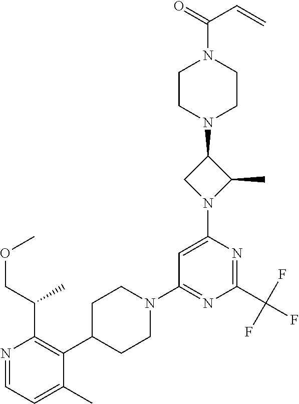 [SMILES: C=CC(=O)N1CCN([C@@H]2CN(c3cc(N4CCC(c5c(C)ccnc5[C@@H](C)COC)CC4)nc(C(F)(F)F)n3)[C@@H]2C)CC1] | 1-(4-((2R,3R)-1-(6- (4-(2-((2R)-1- methoxy-2- propanyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-324 | 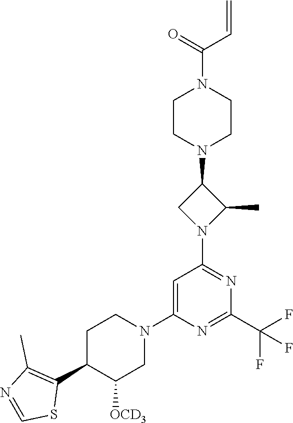 | 1-(4-((2R,3R)-1-(6- ((3R,4S)-3- (methoxy-d3)-4-(4- methylthiazol-5- yl)piperidin-1-yl)- 2-(trifluoromethyl) pyrimidin-4-yl)-2- methylazetidin-3- yl)piperazin-1- yl)prop-2-en-1-one | 2-328 | 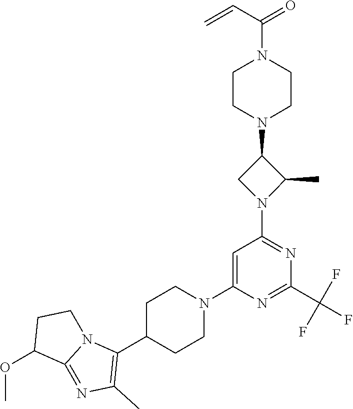 [SMILES: C=CC(=O)N1CCN([C@@H]2CN(c3cc(N4CCC(c5c(C)nc6n5CCC6OC)CC4)nc(C(F)(F)F)n3)[C@@H]2C)CC1] | 1-(4-((2R,3R)-1-(6- (4-(7-methoxy-2- methyl-6,7- dihydro-5H- pyrrolo[1,2- alintidazol-3-yl)-1- piperidinyl)-2- (trifluoromethyl)- 4-pyrimidinyl)-2- methyl-3- azetidinyl)-1- piperazinyl)-2- propen-1-one |
| | mixture of trans isomers at | | | | |
| | piperidine | | | | |
| 2-329 | 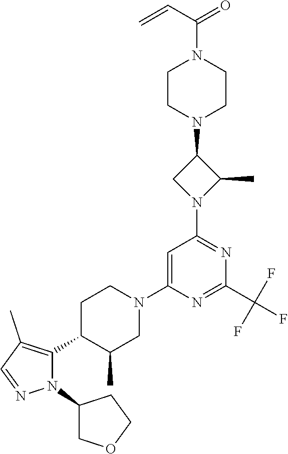 [SMILES: C=CC(=O)N1CCN([C@@H]2CN(c3cc(N4CC[C@@H](c5c(C)cnn5[C@H]5CCOC5)[C@H](C)C4)nc(C(F)(F)F)n3)[C@@H]2C)CC1] | 1-(4-((2R,3R)-2- methyl-1-16- ((3S,4R)-3-methyl- 4-(4-methyl-1- ((3S)-tetrahydro-3- furanyl)-1H- pyrazol-5-yl)-1- piperidinyl)-2- (trifluoromethyl)- 4-pyrimidinyl)-3- azetidinyl)-1- piperazinyl)-2- propen-1-one | 2-333 | 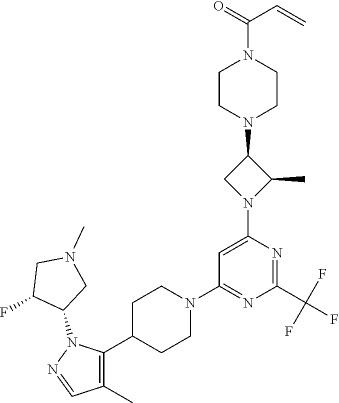 [SMILES: C=CC(=O)N1CCN([C@@H]2CN(c3cc(N4CCC(c5c(C)cnn5[C@H]5CN(C)C[C@H]5F)CC4)nc(C(F)(F)F)n3)[C@@H]2C)CC1] | 1-(4-((2R,3R)-1-(6- (4-(1-((3S.4R)-4- fluoro-1-methyl-3- pyrrolidinyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-330 | 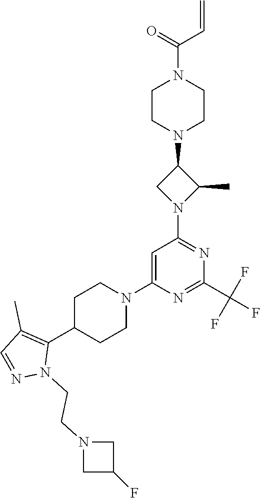 [SMILES: C=CC(=O)N1CCN([C@@H]2CN(c3cc(N4CCC(c5c(C)cnn5CCN5CC(F)C5)CC4)nc(C(F)(F)F)n3)[C@@H]2C)CC1] | 1-(4-((2R,3R)-1-(6- (4-(1-(2-(3-fluoro- 1-azendinyI)ethyl)- 4-methyl-1H- pyrazol-5-yl)-1- piperidiny])-2- (trifluoromethyl)- 4-pyrimidinyl)-2- methyl-3- azetidinyl)-1- piperazinyl)-2- propen-1-one | 2-334 | 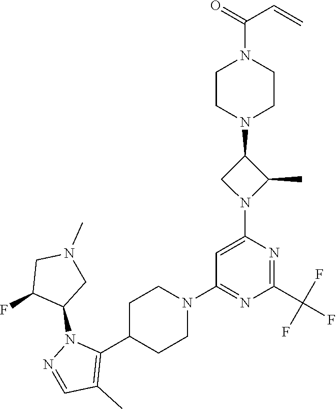 [SMILES: C=CC(=O)N1CCN([C@@H]2CN(c3cc(N4CCC(c5c(C)cnn5[C@@H]5CN(C)C[C@@H]5F)CC4)nc(C(F)(F)F)n3)[C@@H]2C)CC1] | 1-(4-((2R,3R)-1-(6- (4-(1-((3R,4S)-4- fluoro-1-methyl-3- pyrrolidinyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-331 | 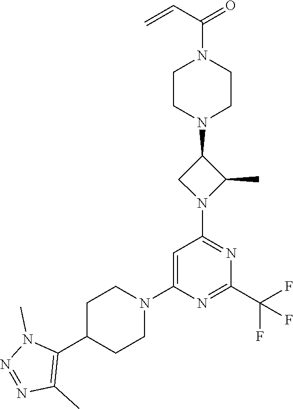 [SMILES: C=CC(=O)N1CCN([C@@H]2CN(c3cc(N4CCC(c5c(C)nnn5C)CC4)nc(C(F)(F)F)n3)[C@@H]2C)CC1] | 1-(4-((2R,3R)-1-(6- (4-(1,4-dimethy]- 1H-1,2,3-triazol-5- yl)-1-piperidinyl)- 2- (trifluoromethyl)- 4-pyrimidinyl)-2- methyl-3- azetidinyl)-1- piperazinyl)-2- propen-1-one | 2-335 | 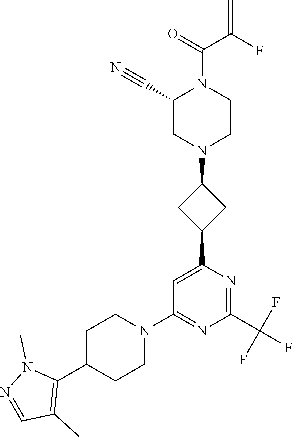 [SMILES: C=C(F)C(=O)N1CCN([C@H]2C[C@@H](c3cc(N4CCC(c5c(C)cnn5C)CC4)nc(C(F)(F)F)n3)C2)C[C@@H]1C#N] | (2R)-4-(cis-3-(6- (4-(1,4-dimethyl- 1H-pyrazol-5-yl)- 1-piperidinyl)-2- (trifluoromethyl)- 4-pyrimidinyl) cyclobutyl)-1- (2-fluoro-2- propenoyl)-2- piperazine- carbonitrile |
| 2-332 | 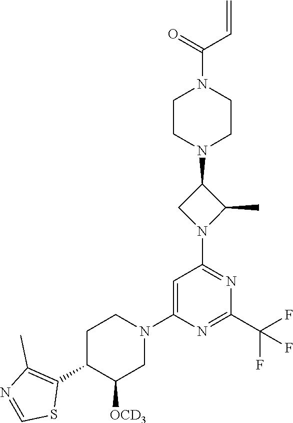 | 1-(4-(2R,3R)-1-(6- ((3S,4R)-3- (methoxy-d3)-4-(4- methylthiazol-5- yl)piperidin-1-yl)- 2- (trifluoromethyl)py rimidin-4-yl)-2- methylazetidin-3- yl)piperazin-1- yl)prop-2-en-1-one | 2-336 | 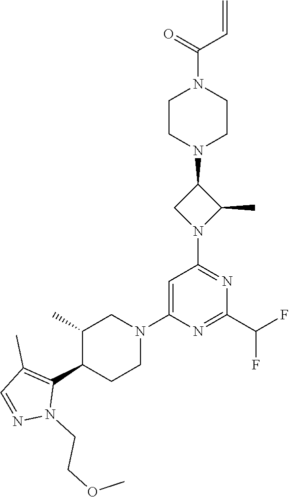 [SMILES: C=CC(=O)N1CCN([C@@H]2CN(c3cc(N4CC[C@@H](c5c(C)cnn5CCOC)[C@H](C)C4)nc(C(F)F)n3)[C@@H]2C)CC1] | 1-(4-((2R,3R)-1-(2- (difluoromethyl)-6- ((3S,4R)-4-(1-(2- methoxyethyl)-4- methyl-1H- pyrazol-5-yl)-3- methyl-1- piperidinyl)-4- pyrimidinyl)-2- methyl-3- azetidinyl)-1- piperazinyl)-2- propen-1-one |
| 2-337 | 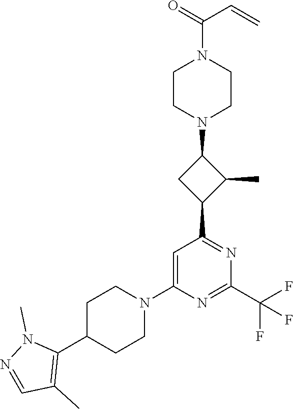 [SMILES: C=CC(=O)N1CCN([C@@H]2C[C@H](c3cc(N4CCC(c5c(C)cnn5C)CC4)nc(C(F)(F)F)n3)[C@@H]2C)CC1] | 1-(4-((1R.25,3S)- 3-(6-(4-(1,4- dimethyl-1H- pyrazol-5-yl)-1- piperidinyl)-2- (trifluoromethyl)- 4-pyrimidinyl)-2- methylcyclobutyl)- [1-piperazinyl)-2- propen-1-one | 2-341 | 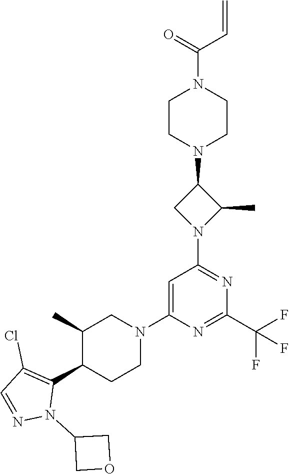 [SMILES: C=CC(=O)N1CCN([C@@H]2CN(c3cc(N4CC[C@@H](c5c(Cl)cnn5C5COC5)[C@@H](C)C4)nc(C(F)(F)F)n3)[C@@H]2C)CC1] | 1-(4-((2R,3R)-1-(6- ((3R.4R)-4-(4- chloro-1-(3- oxetanyl)-1H- pyrazol-5-yl)-3- methyl-1- piperidinyl)-2- (trifluoromcthyl)- 4-pyrimidinyl)-2- methyl-3- azetidinyl)-1- piperazinyl)-2- propen-1-one |
| 2-338 | 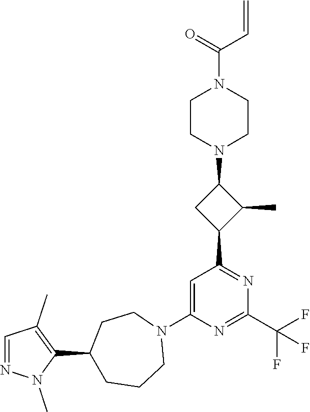 [SMILES: C=CC(=O)N1CCN([C@@H]2C[C@H](c3cc(N4CCC[C@@H](c5c(C)cnn5C)CC4)nc(C(F)(F)F)n3)[C@@H]2C)CC1] | 1-(4-((2R,3R)-1-(6- ((4R)-4-(1,4- dimethyl-1H- pyrazol-5-yl)-1- azepanyl)-2- (trifluoromethyl)- 4-pyrimidinyl)-2- methyl-3- azetidinyl)-1- piperazinyl)-2- propen-1-one | 2-342 | 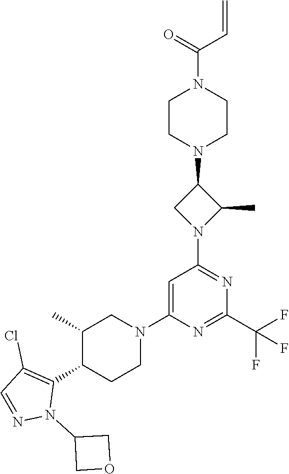 [SMILES: C=CC(=O)N1CCN([C@@H]2CN(c3cc(N4CC[C@H](c5c(Cl)cnn5C5COC5)[C@H](C)C4)nc(C(F)(F)F)n3)[C@@H]2C)CC1] | 1-(4-((2R,3R)-1-(6- ((3S,4S)-4-(4- chloro-1-(3- oxetanyl)-1H- pyrazol-5-yl)-3- methyl-1- piperidinyl)-2- (trifluoromethyl)- 4-pyrintidinyl)-2- methyl-3- azetidinyl)-1- piperazinyl)-2- propen-1-one |
| 2-339 | 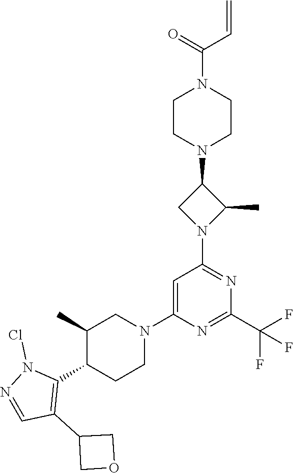 [SMILES: C=CC(=O)N1CCN([C@@H]2CN(c3cc(N4CC[C@H](c5c(C6COC6)cnn5Cl)[C@@H](C)C4)nc(C(F)(F)F)n3)[C@@H]2C)CC1] | 1-(4-((2R,3R)-1-(6- ((3R,4S)-4-(4- chloro-1-(3- oxetanyl)-1H- pyrazol-5-yl)-3- methyl-1- piperidiny])-2- (trifluoromethyl)- 4-pyrimidinyl)-2- methyl-3- azetidinyl)-1- piperazinyl)-2- propen-1-one | 2-343 | 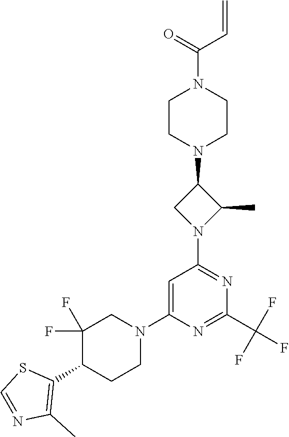 [SMILES: C=CC(=O)N1CCN([C@@H]2CN(c3cc(N4CC[C@H](c5scnc5C)C(F)(F)C4)nc(C(F)(F)F)n3)[C@@H]2C)CC1] | 1-(4-((2R,3R)-1-(6- ((4S)-3,3-difluoro- 4-(4-methy]-1,3- thiazol-5-yl)-1- piperidinyl)-2- (trifluoromethyl)- 4-pyrimidinyl)-2- methyl-3- azetidinyl)-1- piperazinyl)-2- propen-1-one |
| 2-340 | 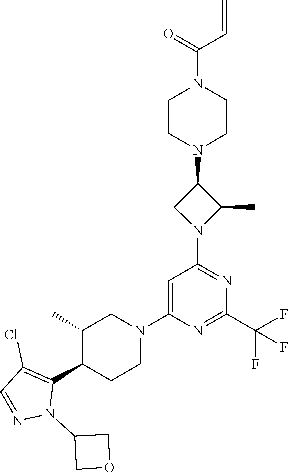 [SMILES: C=CC(=O)N1CCN([C@@H]2CN(c3cc(N4CC[C@@H](c5c(Cl)cnn5C5COC5)[C@H](C)C4)nc(C(F)(F)F)n3)[C@@H]2C)CC1] | 1-(4-((2R,3R)-1-(6- ((3S,4R)-4-(4- chloro-1-(3- oxetanyl)-1H- pyrazol-5-yl)-3- nicthyl-1- piperidinyl)-2- (trifluoromethyl)- 4-pyrimidinyl)-2- methyl-3- azetidinyl)-1- piperazinyl)-2- propen-1-one | 2-344 | 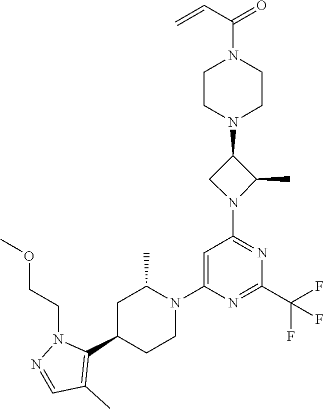 [SMILES: C=CC(=O)N1CCN([C@@H]2CN(c3cc(N4CC[C@@H](c5c(C)cnn5CCOC)C[C@@H]4C)nc(C(F)(F)F)n3)[C@@H]2C)CC1] | 1-(4-((2R,3R)-1-(6- ((2S,4R)-4-(1-(2- methoxyethyl)-4- methyl-1H- pyrazol-5-yl)-2- methyl-1- piperidinyl)-2- (trifluoromethyl)- 4-pyrimidinyl)-2- methyl-3- azetidinyl)-1- piperaziny])-2- propen-1-one |
| 2-345 | 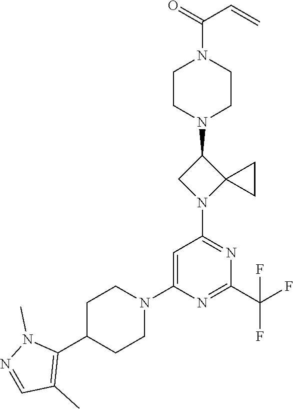 [SMILES: C=CC(=O)N1CCN([C@@H]2CN(c3cc(N4CCC(c5c(C)cnn5C)CC4)nc(C(F)(F)F)n3)C23CC3)CC1] | 1-(4-((6R)-4-(6-(4- (1,4-dimethyl-1H- pyrazol-5-yl)-1- piperidinyl)-2- (trifluoromethyl)- 4-pyrimidinyl)-4- azaspiro[2.3] hexan-6-yl)-1- piperazinyl)-2- propen-1-one | 2-349 | 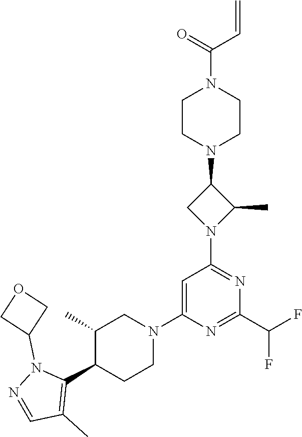 [SMILES: C=CC(=O)N1CCN([C@@H]2CN(c3cc(N4CC[C@@H](c5c(C)cnn5C5COC5)[C@H](C)C4)nc(C(F)F)n3)[C@@H]2C)CC1] | 1-(4-((2R,3R)-1-(2- (difluoromethyl)-6- ((3S,4R)-3-methyl- 4-(4-methyl-1-(3- oxetanyl)-1H- pyrazol-5-yl)-1- piperidinyl)-4- pyrimidinyl)-2- methyl-3- azetidinyl)-1- piperazinyl)-2- propen-1-one |
| 2-346 | 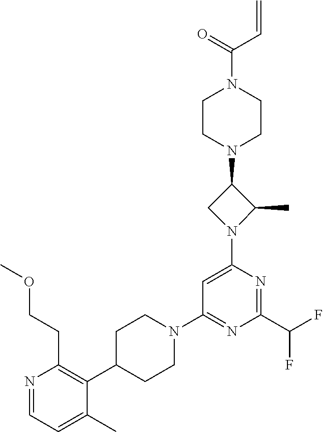 [SMILES: C=CC(=O)N1CCN([C@@H]2CN(c3cc(N4CCC(c5c(C)ccnc5CCOC)CC4)nc(C(F)F)n3)[C@@H]2C)CC1] | 1-(4-((2R,3R)-1-(2- (difluoromcthyl)-6- (4-(2-(2- methoxyethyl)-4- methyl-3- pyridinyl)-1- piperidinyl)-4- pyrimidinyl)-2- methyl-3- azetidinyl)-1- piperazinyl)-2- propen-1-one | 2-350 | 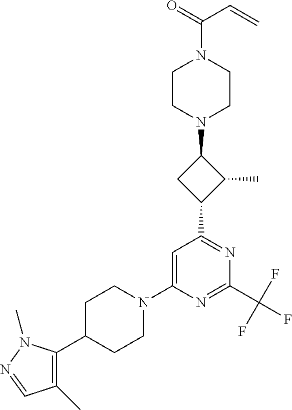 [SMILES: C=CC(=O)N1CCN([C@@H]2C[C@@H](c3cc(N4CCC(c5c(C)cnn5C)CC4)nc(C(F)(F)F)n3)[C@H]2C)CC1] | 1-(4-((IR,2R,3R)- 3-(6-(4-(1,4- dimethyl-1H- pyrazol-5-yl)-1- piperidinyl)-2- (trifluoromethyl)- 4-pyrimidinyl)-2- methylcyclobutyl)- 1-piperazinyl)-2- propeu-1-one |
| 2-347 | 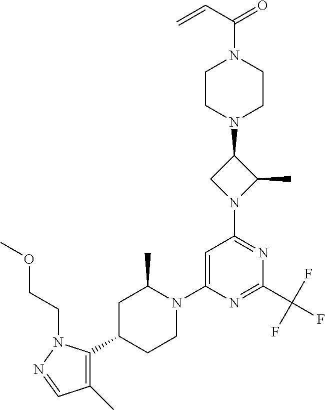 [SMILES: C=CC(=O)N1CCN([C@@H]2CN(c3cc(N4CC[C@H](c5c(C)cnn5CCOC)C[C@H]4C)nc(C(F)(F)F)n3)[C@@H]2C)CC1] | 1-(4-((2R,3R)-1-(6- ((2R,4S-4-(1-(2- methoxyethyl)-4- methyl-1H- pyrazol-5-yl)-2- methyl-1- piperidiny])-2- (trifluoromethyl)- 4-pyrimidinyl)-2- methyl-3- azetidinyl)-1- piperazinyl)-2- propen-1-one | 2-351 | 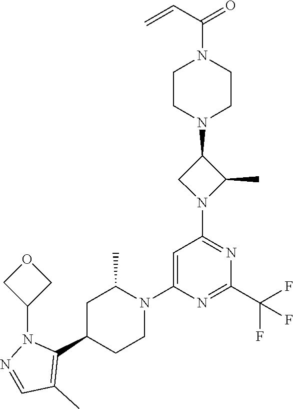 [SMILES: C=CC(=O)N1CCN([C@@H]2CN(c3cc(N4CC[C@@H](c5c(C)cnn5C5COC5)C[C@@H]4C)nc(C(F)(F)F)n3)[C@@H]2C)CC1] | 1-(4-((2R.3R)-2- methyl-1-(6- ((2S,4R)-2-methyl- 4-(4-methyl-1-(3- oxetanyl)-1H- pyrazol-5-yl)-1- piperidinyl)-2- (trifluoromethyl)- 4-pyrimidinyl)-3- azetidinyl)-1- piperazinyl)-2- propen-1-one |
| 2-348 | 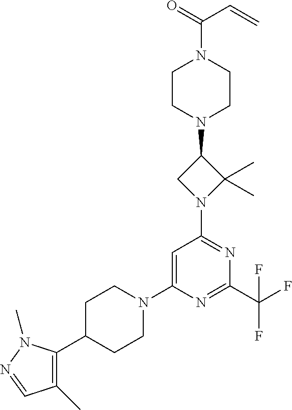 [SMILES: C=CC(=O)N1CCN([C@@H]2CN(c3cc(N4CCC(c5c(C)cnn5C)CC4)nc(C(F)(F)F)n3)C2(C)C)CC1] | 1-(4-((3R)-1-(6-(4- (1,4-dimethyl-1H- pyrazol-5-yl)-1- piperidinyl)-2- (trifluoromethyl)- 4-pyrimidiny])-2,2- dimethyl-3- azetidinyl)-1- piperazinyl)-2- propen-1-one | 2-352 | 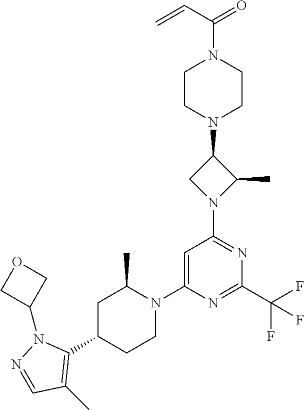 [SMILES: C=CC(=O)N1CCN([C@@H]2CN(c3cc(N4CC[C@H](c5c(C)cnn5C5COC5)C[C@H]4C)nc(C(F)(F)F)n3)[C@@H]2C)CC1] | 1-(4-((2R,3R)-2- methyl-1-(6- ((2R,4S)-2-methyl- 4-(4-methyl-1-(3- oxetanyl)-1H- pyrazol-5-yl)-1- piperidiny])-2- (trifluoromethyl)- 4-pyrimidinyl)-3- azetidinyl)-1- piperazinyl)-2- propen-1-one |
| 2-353 | 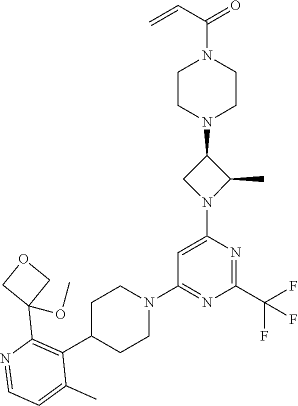 [SMILES: C=CC(=O)N1CCN([C@@H]2CN(c3cc(N4CCC(c5c(C)ccnc5C5(OC)COC5)CC4)nc(C(F)(F)F)n3)[C@@H]2C)CC1] | 1-(4-((2R,3R)-1-(6- (4-(2-(3-methoxy- 3-oxetanyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one | 2-357 | 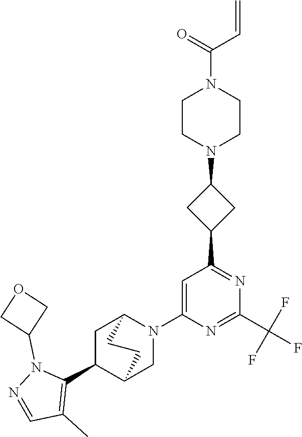 [SMILES: C=CC(=O)N1CCN([C@H]2C[C@@H](c3cc(N4C[C@@H]5CC[C@H]4C[C@@H]5c4c(C)cnn4C4COC4)nc(C(F)(F)F)n3)C2)CC1] | 1-(4-(cis-3-(6- ((1S,4R,5S)-5-(4- methyl-1-(3- oxetanyl)-1H- pyrazol-5-yl)-2- azabicyclo[2.2.2] octan-2-yl)-2- (trifluoromethyl)- 4-pyrimidinyl) cyclobutyl)-1- piperazinyl)-2- propen-1-one |
| 2-354 | 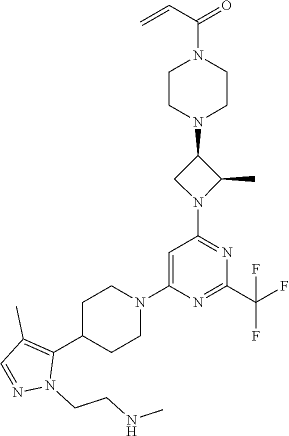 [SMILES: C=CC(=O)N1CCN([C@@H]2CN(c3cc(N4CCC(c5c(C)cnn5CCNC)CC4)nc(C(F)(F)F)n3)[C@@H]2C)CC1] | 1-(4-((2R,3R)-2- methyl-1-(6-(4-(4- methyl-1-(2- (methylamino) ethyl)-1H- pyrazol-5-yl)-1- piperidinyl)-2- (trifluoromethyl)- 4-pyrimidinyl)-3- azetidinyl)-1- piperazinyl)-2- propen-1-one | 2-358 | 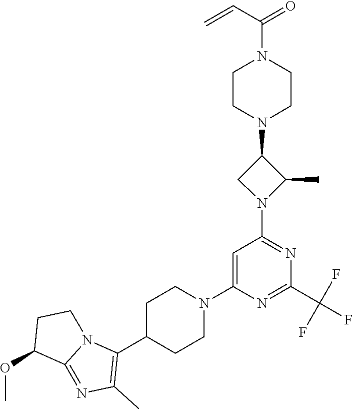 [SMILES: C=CC(=O)N1CCN([C@@H]2CN(c3cc(N4CCC(c5c(C)nc6n5CC[C@@H]6OC)CC4)nc(C(F)(F)F)n3)[C@@H]2C)CC1] | 1-(4-((2R,3R)-1-(6- (4-((75)-7- methoxy-2-methyl- 6,7-dihydro-5H- pyrrolo[1,2- alimidazol-3-yl)-1- piperidinyl)-2- (trifluoromethyl)- 4-pyrintidinyl)-2- methyl-3- azetidinyl)-1- piperazinyl)-2- propen-1-one |
| 2-355 | 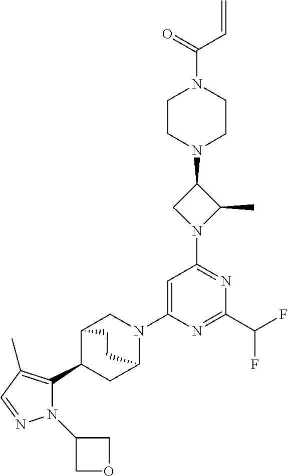 [SMILES: C=CC(=O)N1CCN([C@@H]2CN(c3cc(N4C[C@H]5CC[C@@H]4C[C@H]5c4c(C)cnn4C4COC4)nc(C(F)F)n3)[C@@H]2C)CC1] | 1-(4-((2R,3R)-1-(2- (difluoromethyl)-6- ((1R,4S,5R)-5-(4- methyl-1-(3- oxetanyl)-1H- pyrazol-5-yl)-2- azabicyclo[2.2.2] octan-2-yl)-4- pyrimidinyl)-2- methyl-3- azetidinyl-1- piperazinyl)-2- propen-1-one | 2-359 | 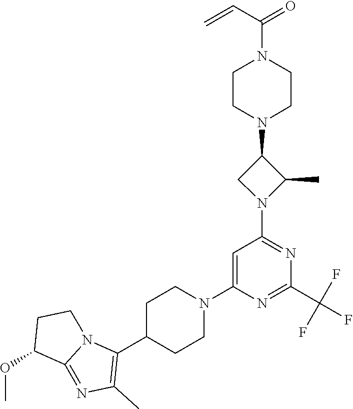 [SMILES: C=CC(=O)N1CCN([C@@H]2CN(c3cc(N4CCC(c5c(C)nc6n5CC[C@H]6OC)CC4)nc(C(F)(F)F)n3)[C@@H]2C)CC1] | 1-(4-((2R,3R)-1-(6- (4-((7R)-7- methoxy-2-methyl- 6,7-dihydro-SH- pyrrolo[1,2- ajimidazol-3-yl)-1- piperidinyl)-2- (trifluoromethyl)- 4-pyrimidinyl)-2- methyl-3- azetidinyl)-1- piperazinyl)-2- propen-1-one |
| 2-356 | 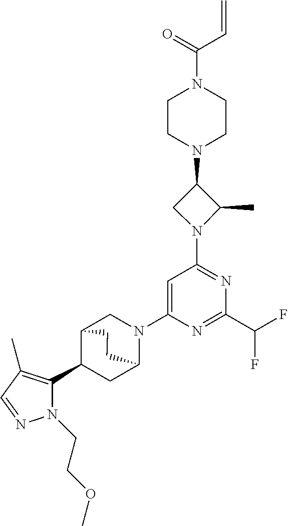 [SMILES: C=CC(=O)N1CCN([C@@H]2CN(c3cc(N4C[C@H]5CC[C@@H]4C[C@H]5c4c(C)cnn4CCOC)nc(C(F)F)n3)[C@@H]2C)CC1] | 1-(4-((2R,3R)-1-(2- (difluoromethyl)-6- ((1R,4S,5R)-5-(1- (2-methoxyethyl)- 4-methyl-1H- pyrazol-5-yl)-2- azabicyclo[2.2.2] octan-2-yl)-4- pyrimidinyl)-2- methyl-3- azetidinyl)-1- piperazinyl)-2- propen-1-one | 2-360 | 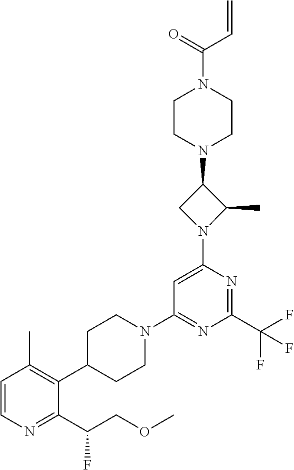 [SMILES: C=CC(=O)N1CCN([C@@H]2CN(c3cc(N4CCC(c5c(C)ccnc5[C@@H](F)COC)CC4)nc(C(F)(F)F)n3)[C@@H]2C)CC1] | 1-(4-((2R,3R)-1-(6- (4-(2-((1R)-1- fluoro-2- methoxyethyl)-4- methyl-3- pyridinyl)-1- piperidinyl)-2- (trifluoromethyl)- 4-pyrimidinyl)-2- methyl-3- azetidinyl)-1- piperaziny])-2- propen-1-one |
| 2-361 | 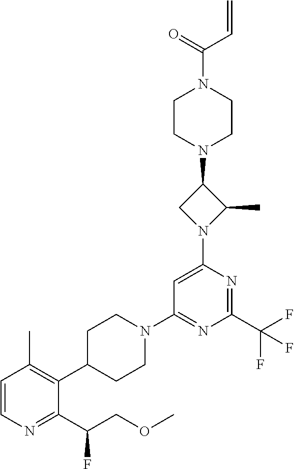 [SMILES: C=CC(=O)N1CCN([C@@H]2CN(c3cc(N4CCC(c5c(C)ccnc5[C@H](F)COC)CC4)nc(C(F)(F)F)n3)[C@@H]2C)CC1] | 1-(4-((2R,3R)-1-(6- (4-(2-((1S)-1- fluoro-2- methoxyethyl)-4- methyl-3- pyridinyl)-1- piperidinyl)-2- (trifluoromcthyl)- 4-pyrimidinyl)-2- methyl-3- azetidinyl)-1- piperazinyl)-2- propen-1-one | 2-365 | 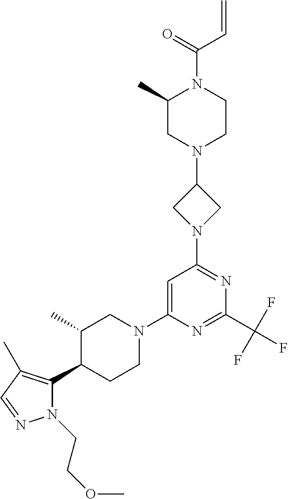 [SMILES: C=CC(=O)N1CCN(C2CN(c3cc(N4CC[C@@H](c5c(C)cnn5CCOC)[C@H](C)C4)nc(C(F)(F)F)n3)C2)C[C@H]1C] | 1-((2R)-4-(1-(6- ((3S,4R)-4-(1-(2- metboxyethyl)-4- methyl-1H- pyrazol-5-yl)-3- methyl-1- piperidinyl)-2- (trifluoromethyl)- 4-pyrimidiny])-3- azetidinyl)-2- methyl-1- piperazinyl)-2- propen-1-one |
| 2-362 | 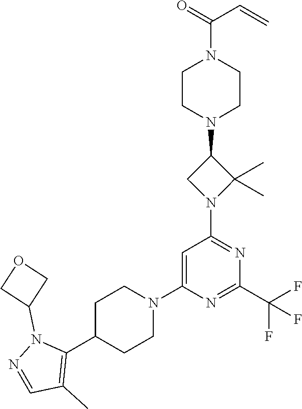 [SMILES: C=CC(=O)N1CCN([C@@H]2CN(c3cc(N4CCC(c5c(C)cnn5C5COC5)CC4)nc(C(F)(F)F)n3)C2(C)C)CC1] | 1-(4-((3R)-2.2- dimethyl-1-(6-(4- (4-methyl-1-(3- oxetanyl)-1H- pyrazol-5-yl)-1- piperidinyl)-2- (trifluoromethyl)- 4-pyrimidinyl)-3- azetidinyl)-1- piperazinyl)-2- propen-1-one | 2-366 | 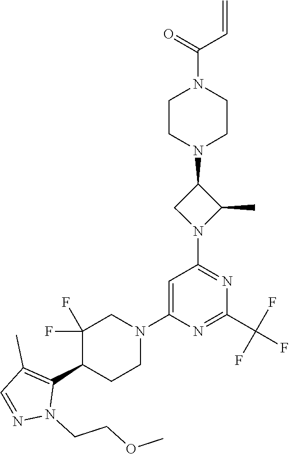 [SMILES: C=CC(=O)N1CCN([C@@H]2CN(c3cc(N4CC[C@@H](c5c(C)cnn5CCOC)C(F)(F)C4)nc(C(F)(F)F)n3)[C@@H]2C)CC1] | 1-(4-((2R,3R)-1-(6- ((4S)-3,3-difluoro- 4-(1-(2- methoxyethyl)-4- methyl-1H- pyrazol-5-yl)-1- piperidinyl)-2- (trifluoromethyl)- 4-pyrimidinyl)-2- methyl-3- azetidinyl)-]- piperazinyl)-2- propen-1-one |
| 2-363 | 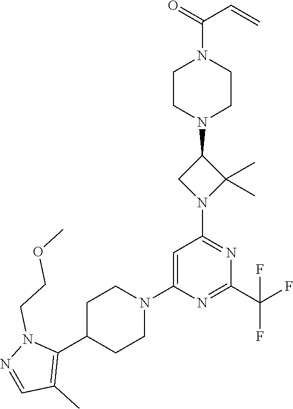 [SMILES: C=CC(=O)N1CCN([C@@H]2CN(c3cc(N4CCC(c5c(C)cnn5CCOC)CC4)nc(C(F)(F)F)n3)C2(C)C)CC1] | 1-(4-((3R)-1-(6-(4- (1-(2- methoxyethyl)-4- methyl-1H- pyrazol-5-yl)-1- piperidinyl)-2- (trifluoromethyl)- 4-pyrimidinyl)-2,2- dimethyl-3- azetidinyl)-1- piperazinyl)-2- propen-1-one | 2-367 | 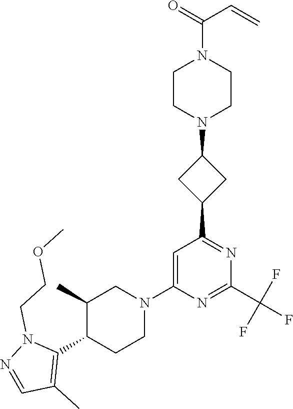 [SMILES: C=CC(=O)N1CCN([C@H]2C[C@@H](c3cc(N4CC[C@H](c5c(C)cnn5CCOC)[C@@H](C)C4)nc(C(F)(F)F)n3)C2)CC1] | 1-(4-(cis-3-(6- ((3R,4S)-4-(1-(2- methoxyethyl)-4- methyl-1H- pyrazol-5-yl)-3- methyl-1- piperidinyl)-2- (trifluoromethyl)- 4-pyrimidinyl) cyclobutyl)-1- piperazinyl)-2- propen-1-one |
| 2-364 | 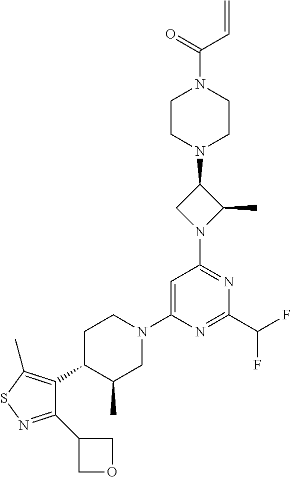 [SMILES: C=CC(=O)N1CCN([C@@H]2CN(c3cc(N4CC[C@@H](c5c(C6COC6)nsc5C)[C@H](C)C4)nc(C(F)F)n3)[C@@H]2C)CC1] | 1-(4-((2R,3R)-1-(2- (difluoromethyl)-6- ((3S,4R)-3-methyl- 4-(5-methyl-3-(3- oxetanyl)-1,2- thiazol-4-yl)-1- piperidiny])-4 pyrimidinyl)-2- methyl-3- azetidinyl)-1- piperazinyl)-2- propen-1-one | 2-368 | 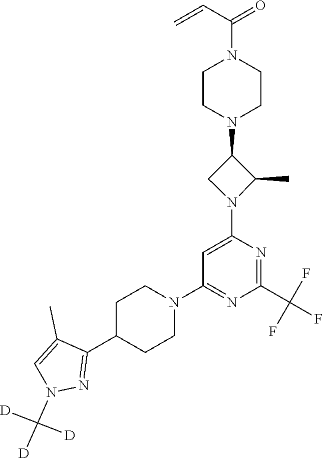 [SMILES: [2H]C([2H])([2H])n1cc(C)c(C2CCN(c3cc(N4C[C@@H](N5CCN(C(=O)C=C)CC5)[C@H]4C)nc(C(F)(F)F)n3)CC2)n1] | 1-(4-((2R,3R)-2- methyl-1-(6-(4-(4- methyl-1- (-2-H_3_)methyl- 1H-pyrazol-3-yl)- 1-piperidinyl)-2- (trifluoromethyl)- 4-pyrimidinyl)-3- azetidinyl)-1- piperazinyl)-2- propen-t-one |
| 2-369 | 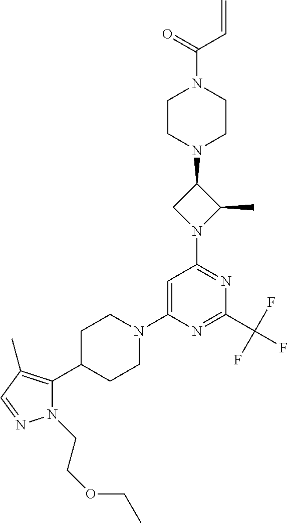 [SMILES: C=CC(=O)N1CCN([C@@H]2CN(c3cc(N4CCC(c5c(C)cnn5CCOCC)CC4)nc(C(F)(F)F)n3)[C@@H]2C)CC1] | 1-(4-((2R,3R)-1-(6- (4-(1-(2- ethoxyethyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-373 | 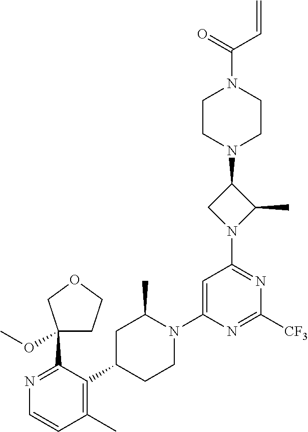 [SMILES: C=CC(=O)N1CCN([C@@H]2CN(c3cc(N4CC[C@H](c5c(C)ccnc5[C@]5(OC)CCOC5)C[C@H]4C)nc(C(F)(F)F)n3)[C@@H]2C)CC1] | 1-(4-((2R,3R)-1-(6- ((2R,4S)-4-(2-((R)- 3-Methoxytetra- hydrofuran-3-yl)-4- methylpyridin-3- yl}-2- methylpiperidin-1- yl)-2- (trifluoromethyl) pyrimidin-4-yl)-2- methylazetidin-3- yl)piperazin-1- yl)prop-2-en-1-one |
| 2-370 | 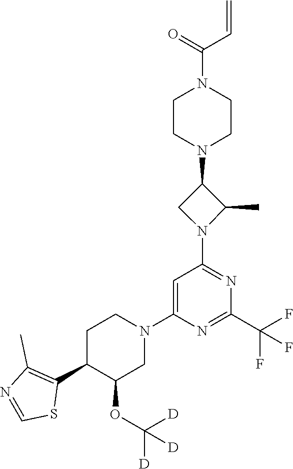 [SMILES: [2H]C([2H])([2H])O[C@@H]1CN(c2cc(N3C[C@@H](N4CCN(C(=O)C=C)CC4)[C@H]3C)nc(C(F)(F)F)n2)CC[C@@H]1c1scnc1C] | 1-(4-((2R,3R)-2- methyl-1-(6- ((3R,4R)-3- ((H_3_) methyloxy)- 4-(4-methyl-1,3- thiazol-5-yl)-1- piperidinyl)-2- (trifluoromethyl)- 4-pyrimidinyl)-3- azetidinyl)-1- piperazinyl)-2- propen-1-one | 2-374 | 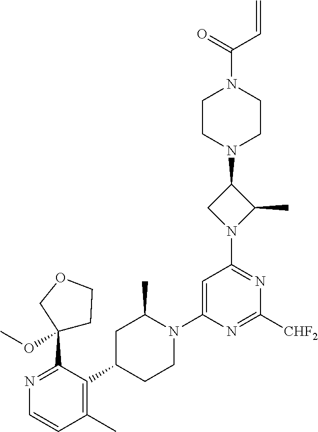 [SMILES: C=CC(=O)N1CCN([C@@H]2CN(c3cc(N4CC[C@H](c5c(C)ccnc5[C@]5(OC)CCOC5)C[C@H]4C)nc(C(F)F)n3)[C@@H]2C)CC1] | 1-(4-((2R,3R)-1-(2- (Difluoromethyl)- 6-((2R,4S)-4-(2- ((R)-3- methoxytetrahydro furan-3-yl)-4- methylpyridin-3- yl)-2- methylpiperidin-1- yl)pyrimidin-4-yl)- 2-methylazetidin- 3-yl)piperazin-1- yl)prop-2-en-1-one |
| 2-371 | 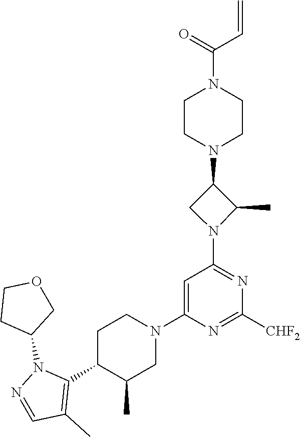 [SMILES: C=CC(=O)N1CCN([C@@H]2CN(c3cc(N4CC[C@@H](c5c(C)cnn5[C@@H]5CCOC5)[C@H](C)C4)nc(C(F)F)n3)[C@@H]2C)CC1] | 1-(4-((2R,3R)-1-(2- (Difluoromethyl)- 6-((3,4R)-3- methyl-4-(4- methyl-1-((S)- tetrahydrofuran-3- yl)-1H-pyrazol-5- yl)piperidin-1- yl)pyrimidin-4-yl)- 2-methylazetidin- 3-yl)piperazin-1- yl)prop-2-en-1-one | 2-375 | 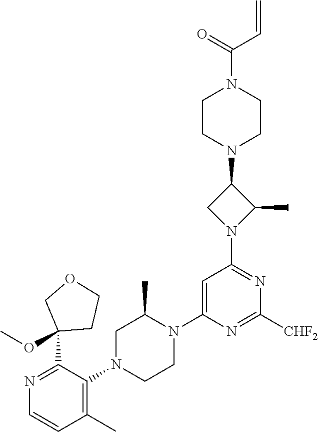 | 1-(4-((2R,3R)-1-(2- (difluoromethyl)-6- ((2R,4S)-4-(2-((S)- 3. methoxytetrahydro furan-3-yl)-4- methylpyridin-3- yl)-2- methylpiperidin-1- yl)pyrimidin-4-yl)- 2-methylazetidin- 3-yl)piperazin-1- yl)prop-2-en-1-one |
| 2-372 | 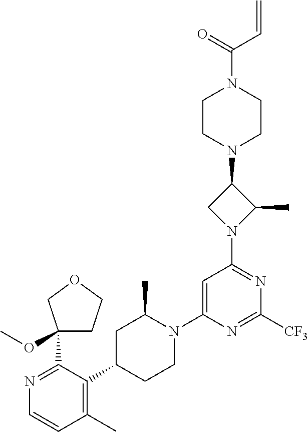 [SMILES: C=CC(=O)N1CCN([C@@H]2CN(c3cc(N4CC[C@H](c5c(C)ccnc5[C@@]5(OC)CCOC5)C[C@H]4C)nc(C(F)(F)F)n3)[C@@H]2C)CC1] | 1-(4-((2R,3R)-1-(6- ((2R,4S)-4-(2-((S)- 3- Methoxytetrahydro furan-3-yl)-4- methylpyridin-3- yl)-2- methylpiperidin-1- yl)-2- (trifluoromethyl) pyrimidin-4-yl)-2- methylazetidin-3- yl)piperazin-1- yl)prop-2-en-1-one | 2-376 | 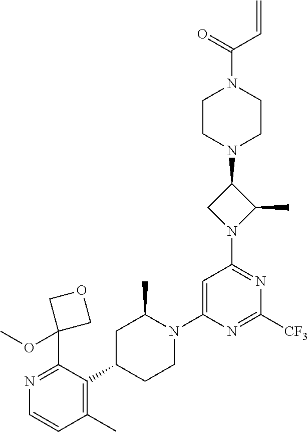 [SMILES: C=CC(=O)N1CCN([C@@H]2CN(c3cc(N4CC[C@H](c5c(C)ccnc5C5(OC)COC5)C[C@H]4C)nc(C(F)(F)F)n3)[C@@H]2C)CC1] | 1-(4-((2R,3R)-1-(6- ((2R,4S)-4-(2-(3- methoxyoxetan-3- yl)-4- methylpyridin-3- yl)-2- methylpiperidin-1- yl)-2- (trifluoromethyl) pyrimidin-4-yl)-2- methylazetidin-3- yl)piperazin-1- yl)prop-2-en-1-one |
| 2-377 | 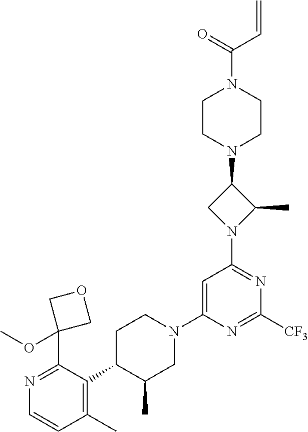 [SMILES: C=CC(=O)N1CCN([C@@H]2CN(c3cc(N4CC[C@@H](c5c(C)ccnc5C5(OC)COC5)[C@H](C)C4)nc(C(F)(F)F)n3)[C@@H]2C)CC1] | 1-(4-((2R,3R)-1-(6- ((3S,4R)-4-(2-(3- methoxyoxetan-3- yl)-4- methylpyridin-3- yl)-3- methylpiperidin-1- yl)-2- (trifluoromethyl) pyrimidin-4-yl)-2- methylazetidin-3- yl)piperazin-1- yl)prop-2-en-1-one | 2-379 | 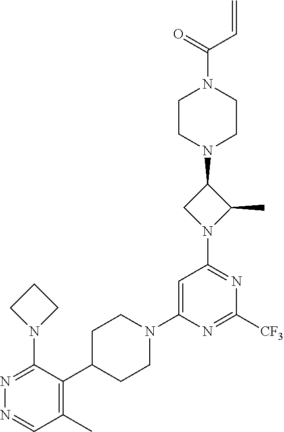 [SMILES: C=CC(=O)N1CCN([C@@H]2CN(c3cc(N4CCC(c5c(C)cnnc5N5CCC5)CC4)nc(C(F)(F)F)n3)[C@@H]2C)CC1] | 1-(4-((2R,3R)-1-(6- (4-(3-(1- azetidinyl)-5- methyl-4- pyridazinyl)-1- piperidinyl)-2- (trifluoromethyl)- 4-pyrimidinyl)-2- methyl-3- azetidinyl)-1- piperazinyl)-2- propen-1-one |
| 2-378 | 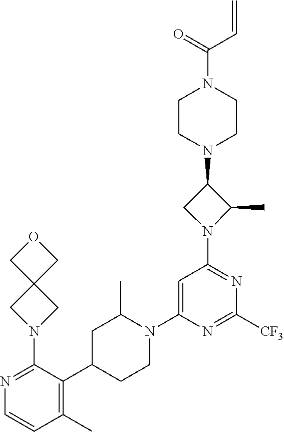 [SMILES: C=CC(=O)N1CCN([C@@H]2CN(c3cc(N4CCC(c5c(C)ccnc5N5CC6(COC6)C5)CC4C)nc(C(F)(F)F)n3)[C@@H]2C)CC1] | 1-(4-((2R,3R)-2- methyl-1-(6-(2- methyl-4-(4- methyl-2-(2-oxa-6- azaspiro[3.3] heptan-6- yl)pyridin-3- yl)piperidin-1-yl)- 2-(trifluoromethyl) pyrimidin-4- ylazetidin-3- yl)piperazin-1- yl)prop-2-en-1-one | 2-380 | 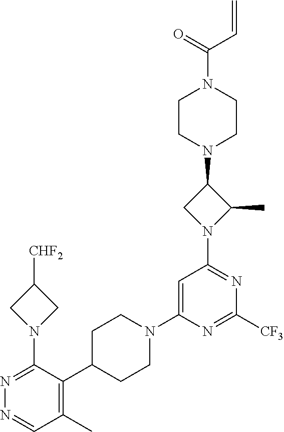 [SMILES: C=CC(=O)N1CCN([C@@H]2CN(c3cc(N4CCC(c5c(C)cnnc5N5CC(C(F)F)C5)CC4)nc(C(F)(F)F)n3)[C@@H]2C)CC1] | 1-(4-((2R,3R)-1-(6- (4-(3-(3- (difluoromethyl)-1- azetidinyl)-5- methyl-4- pyridazinyl)-1- piperidinyl)-2- (trifluoromethyl)- 4-pyrimidinyl)-2- methyl-3- azetidinyl)-1- piperazinyl)-2- propen-1-one |
| 2-378-1 | 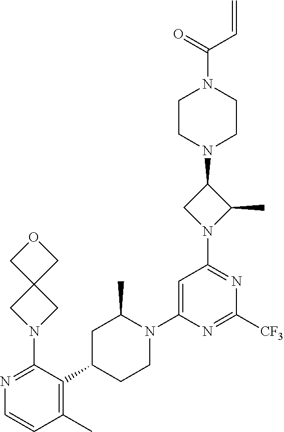 [SMILES: C=CC(=O)N1CCN([C@@H]2CN(c3cc(N4CC[C@H](c5c(C)ccnc5N5CC6(COC6)C5)C[C@H]4C)nc(C(F)(F)F)n3)[C@@H]2C)CC1] | 1-(4-((2R,3R)-2- methyl-1-16- ((2R,4S)-2-methyl- 4-(4-methyl-2-(2- oxa-6- azaspiro[3.3] heptan-6- yl)pyridin-3- yl)piperidin-1-yl)- 2-(trifluoromethyl) pyrimidin-4- yl)azetidin-3- yl)piperazin-1- yl)prop-2-en-1-one | 2-382 | 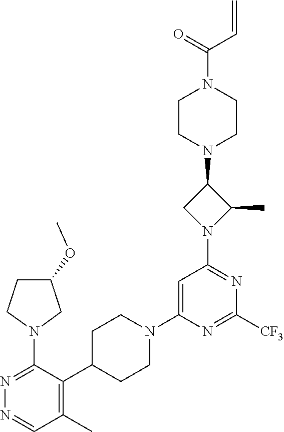 [SMILES: C=CC(=O)N1CCN([C@@H]2CN(c3cc(N4CCC(c5c(C)cnnc5N5CC[C@H](OC)C5)CC4)nc(C(F)(F)F)n3)[C@@H]2C)CC1] | 1-(4-((2R,3R)-1-(6- (4-(3-((3S)-3- methoxy-1- pyrrolidinyl)-5- methyl-4- pyridaziny])-1- piperidinyl)-2- (trifluoromethyl)- 4-pyrimidinyl)-2- methyl-3- azetidinyl)-1- piperazinyl)-2- propen-1-one |
| | 2^{nd}-eluting isomer | | | | |
| 2-378-2 | 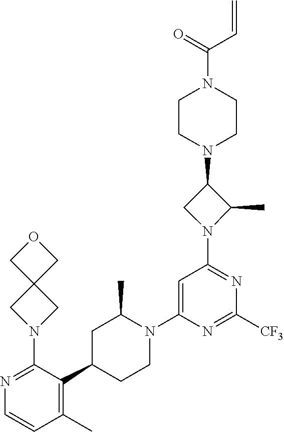 [SMILES: C=CC(=O)N1CCN([C@@H]2CN(c3cc(N4CC[C@@H](c5c(C)ccnc5N5CC6(COC6)C5)C[C@H]4C)nc(C(F)(F)F)n3)[C@@H]2C)CC1] | 1-(4-((2R,3R)-2- methyl-1-(6- ((2R,4R)-2-methyl- 4-(4-methyl-2-(2- oxa-6- azaspiro[3.3] heptan-6-yl)-3- pyridinyl)- 1-piperidinyl)-2- (trifluoromethyl)- 4-pyrimidinyl)-3- azetidinyl)-1- piperazinyl)-2- propen-1-one | 2-383 | 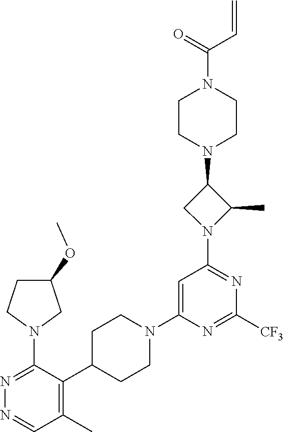 [SMILES: C=CC(=O)N1CCN([C@@H]2CN(c3cc(N4CCC(c5c(C)cnnc5N5CC[C@@H](OC)C5)CC4)nc(C(F)(F)F)n3)[C@@H]2C)CC1] | 1-(4-((2R,3R)-1-(6- (4-(3-((3R)-3- methoxy-1- pyrrolidinyl)-5- methyl-4- pyridaziny])-1- piperidinyl)-2- (trifluoromethyl)- 4-pyrimidinyl)-2- methyl-3- azetidinyl)-1- piperazinyl)-2- propen-1-one |
| | 1^{st}-eluting isomer | | | | |
| 2-384 | 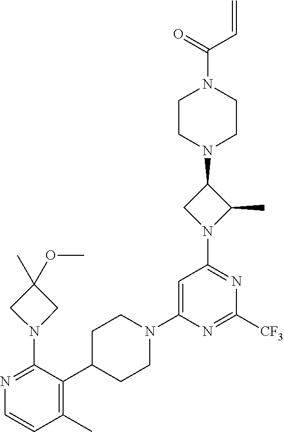 [SMILES: C=CC(=O)N1CCN([C@@H]2CN(c3cc(N4CCC(c5c(C)ccnc5N5CC(C)(OC)C5)CC4)nc(C(F)(F)F)n3)[C@@H]2C)CC1] | 1-(4-((2R,3R)-1-(6- (4-(2-(3-methoxy- 3-methyl-1- azetidinyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one | 2-388 | 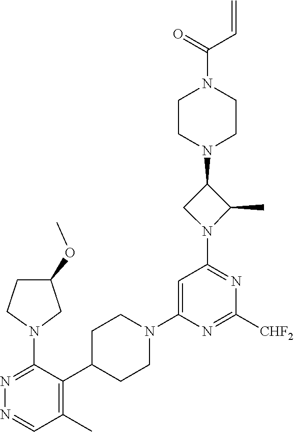 [SMILES: C=CC(=O)N1CCN([C@@H]2CN(c3cc(N4CCC(c5c(C)cnnc5N5CC[C@@H](OC)C5)CC4)nc(C(F)F)n3)[C@@H]2C)CC1] | 1-(4-((2R,3R)-1-(2- (difluoromethyl)-6- (4-(3-((3R)-3- methoxy-1- pyrrolidinyl)-5- methyl-4- pyridazinyl)-1- pipcridinyl)-4- pyrimidinyl)-2- methyl-3- azetidinyl)-1- piperazinyl)-2- propen-1-one |
| 2-385 | 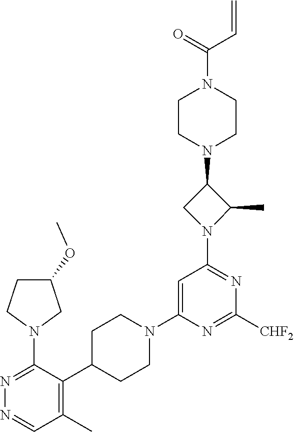 [SMILES: C=CC(=O)N1CCN([C@@H]2CN(c3cc(N4CCC(c5c(C)cnnc5N5CC[C@H](OC)C5)CC4)nc(C(F)F)n3)[C@@H]2C)CC1] | 1-(4-((2R,3R)-1-(2- (difluoromethyl)-6- (4-(3-((38)-3- methoxy-1- pyrrolidinyl)-5- methyl-4- pyridazinyl)-1- piperidinyl)-4- pyrimidinyl)-2- methyl-3- azetidinyl)-1- piperazinyl)-2- propen-1-one | 2-389 | 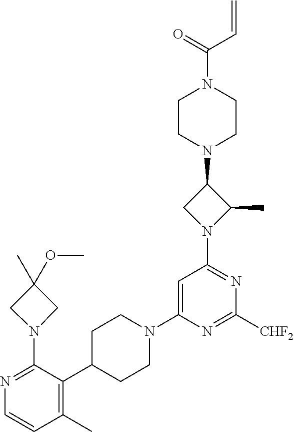 [SMILES: C=CC(=O)N1CCN([C@@H]2CN(c3cc(N4CCC(c5c(C)ccnc5N5CC(C)(OC)C5)CC4)nc(C(F)F)n3)[C@@H]2C)CC1] | 1-(4-((2R,3R)-1-(2- (difluoromethyl)-6- (4-(2-(3-methoxy- 3-methyl-1- azetidinyl)-4- methyl-3- pyridinyl)-1- piperidinyl)-4- pyrimidinyl)-2- methyl-3- azetidinyl)-]- piperazinyl)-2- propen-1-one |
| 2-386 | 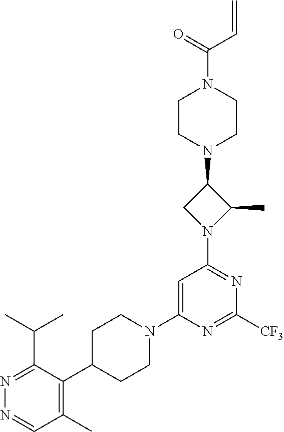 [SMILES: C=CC(=O)N1CCN([C@@H]2CN(c3cc(N4CCC(c5c(C)cnnc5C(C)C)CC4)nc(C(F)(F)F)n3)[C@@H]2C)CC1] | 1-(4-((2R,3R)-2- methyl-1-(6-(4-(5- methyl-3-(2- propanyl)-4- pyridazinyl)-1- piperidiny])-2- (trifluoromethyl)- 4-pyrimidinyl)-3- azetidinyl)-1- piperazinyl)-2- propen-1-one | 2-390 | 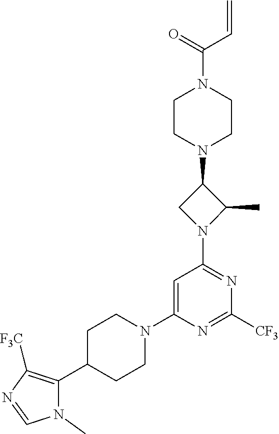 [SMILES: C=CC(=O)N1CCN([C@@H]2CN(c3cc(N4CCC(c5c(C(F)(F)F)ncn5C)CC4)nc(C(F)(F)F)n3)[C@@H]2C)CC1] | 1-(4-((2R,3R)-2- methyl-1-(6-(4-(1- methyl-4- (trifluoromethyl)- [1H-imidazol-5-yl)- 1-piperidinyl)-2- (trifluoromethyl)- 4-pyrimidinyl)-3- azetidinyl)-1- piperazinyl)-2- propen-1-one |
| 2-387 | 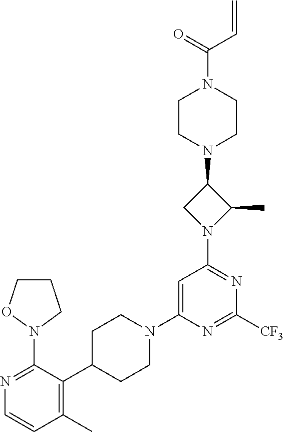 [SMILES: C=CC(=O)N1CCN([C@@H]2CN(c3cc(N4CCC(c5c(C)ccnc5N5CCCO5)CC4)nc(C(F)(F)F)n3)[C@@H]2C)CC1] | 1-(4-((2R,3R)-2- methyl-1-(6-(4-(4- methyl-2-(1,2- oxazolidin-2-yl)-3- pyridinyl)-1- piperidinyl)-2- (trifluoromethyl)- 4-pyrimidinyl)-3- azetidinyl)-1- piperazinyl)-2- propen-1-one | 2-391 | 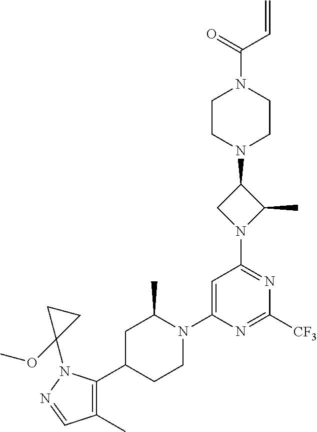 [SMILES: C=CC(=O)N1CCN([C@@H]2CN(c3cc(N4CCC(c5c(C)cnn5C5(OC)CC5)C[C@H]4C)nc(C(F)(F)F)n3)[C@@H]2C)CC1] | 1-(4-((2R,3R)-1-(6- ((2R)-4-(1-(1- methoxycyclo- propyl)-4- methyl-1H- pyrazol-5-yl)-2- methylpiperidin-1- yl)-2- (trifluoromethyl) pyrimidin-4-yl)-2- methylazetidin-3- yl)ypiperazin-1- yl)prop-2-en-1-one |
| 2-391-1 | 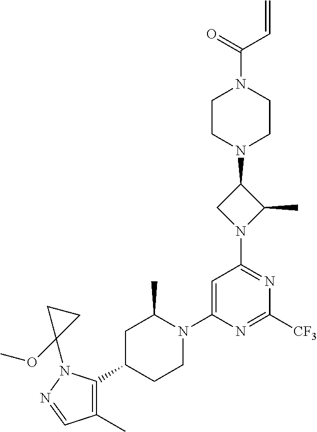 [SMILES: C=CC(=O)N1CCN([C@@H]2CN(c3cc(N4CC[C@H](c5c(C)cnn5C5(OC)CC5)C[C@H]4C)nc(C(F)(F)F)n3)[C@@H]2C)CC1] | 1-(4-((2R,3R)-1-(6- ((2R,4S)-4-(1-(1- methoxycyclo- propyl)-4- methyl-1H- pyrazol-5-yl)-2- methyl-1- piperidinyl)-2- (trifluoromethyl)- 4-pyrimidinyl)-2- methyl-3- azetidinyl)-1- piperazinyl)-2- propen-1-one | 2-394 | 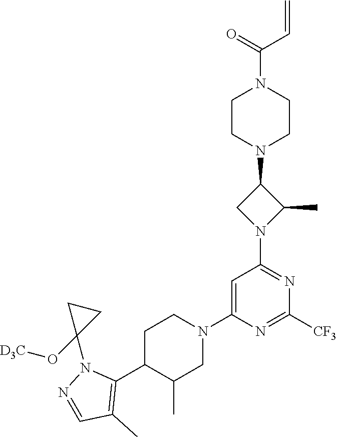 [SMILES: [2H]C([2H])([2H])OC1(n2ncc(C)c2C2CCN(c3cc(N4C[C@@H](N5CCN(C(=O)C=C)CC5)[C@H]4C)nc(C(F)(F)F)n3)CC2C)CC1] | 1-(4-((2R,3R)-1-(6- (4-(1-(1-(methoxy- d3)cyclopropyl)-4- methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one |
| | 2^{nd}-eluting isomer | | | | |
| 2-392 | 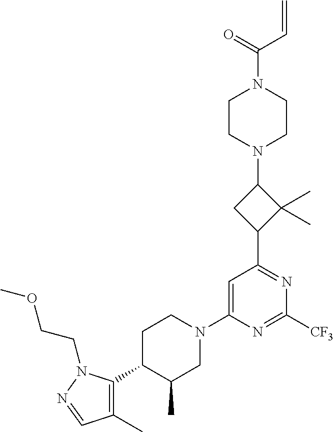 [SMILES: C=CC(=O)N1CCN(C2CC(c3cc(N4CC[C@@H](c5c(C)cnn5CCOC)[C@H](C)C4)nc(C(F)(F)F)n3)C2(C)C)CC1] | 1-(4-((1R,3S)-3-(6- ((3S,4R)-4-(1-(2- methoxyethyl)-4- methyl-1H- pyrazol-5-yl)-3- methyl-1- piperidinyl)-2- (trifluoromethyl)- 4-pyrimidinyl)-2,2- dimethylcyclo- butyl)-1- piperazinyl)-2- propen-1-one | 2-394-1 | 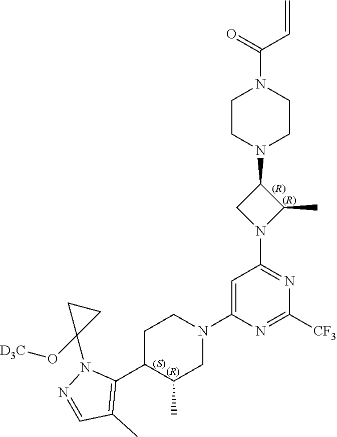 | 1-(4-((2R,3R)-1-(6- ((3R.4S)-4-(1-(1- (methoxy- d₃)cyclopropyl)-4- methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one |
| | | | | 1^{st}-eluting isomer | |
| 2-392-1 | 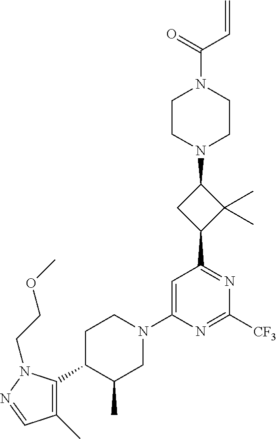 [SMILES: C=CC(=O)N1CCN([C@@H]2C[C@H](c3cc(N4CC[C@@H](c5c(C)cnn5CCOC)[C@H](C)C4)nc(C(F)(F)F)n3)C2(C)C)CC1] | 1-(4-((1R,3S)-3-(6- ((3S,4R)-4-(1-(2- methoxyethyl)-4- methyl-1H- pyrazol-5-yl)-3- methyl-1- piperidinyl)-2- (trifluoromethyl)- 4-pyrimidinyl)-2,2- dimethylcyclo- butyl)-1- piperazinyl)-2- propen-1-one | 2-394-2 | 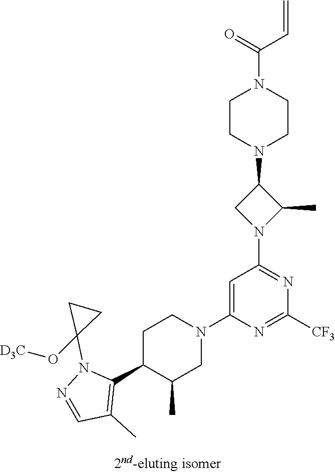 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)(F)F)n3)C[C@H]2C)CC1] | 1-(4-((2R,3R)-1-(6- ((3S,4S)-4-(1-(1- (methoxy- d₃)cyclopropyl)-4- methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one |
| | 1^{st}-eluting isomer | | | | |
| 2-393 | 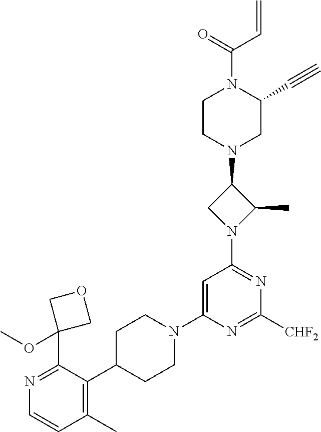 [SMILES: C#C[C@@H]1CN([C@@H]2CN(c3cc(N4CCC(c5c(C)ccnc5C5(OC)COC5)CC4)nc(C(F)F)n3)[C@@H]2C)CCN1C(=O)C=C] | 1-((2R)-4- ((2R,3R)-1-(2- (difluoromethyl)-6- (4-(2-(3-methoxy- 3-oxetanyl)-4- methyl-3- pyridinyl)-1- piperidinyl)-4- pyrimidinyl)-2- methyl-3- azetidinyl)-2- ethynyl-1- piperazinyl)-2- propen-1-one | 2-394-3 | 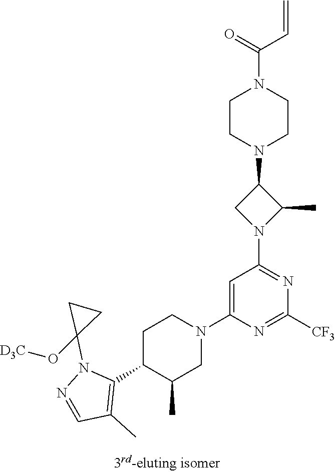 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)(F)F)n3)C[C@H]2C)CC1] | 1-(4-((2R,3R)-1-(6- ((3S,4R)-4-(1-(1- (methoxy- d₃)cyclopropyl)-4- methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one |
| 2-394-4 | 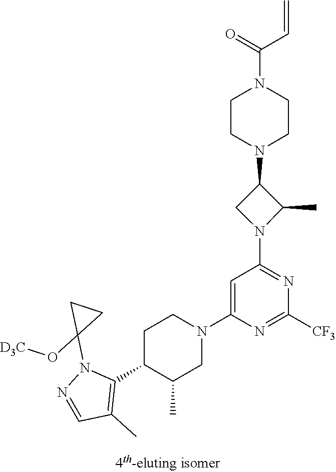 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)(F)F)n3)C[C@@H]2C)CC1] | 1-(4-((2R,3R)-1-(6- ((3R,4R)-4-(1-(1- (methoxy- d₃)cyclopropyl)-4- methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one | 2-396 | 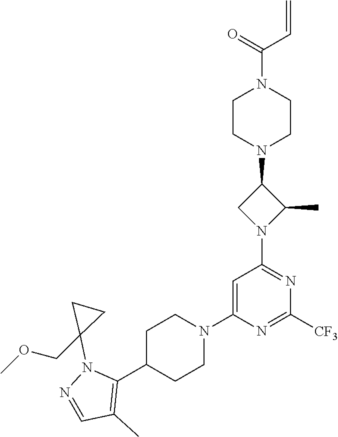 [SMILES: C=CC(=O)N1CCN([C@@H]2CN(c3cc(N4CCC(c5c(C)cnn5C5(COC)CC5)CC4)nc(C(F)(F)F)n3)[C@@H]2C)CC1] | 1-(4-((2R,3R)-1-(6- (4-(1-(1- (methoxymethyl) cyclopropyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-395 | 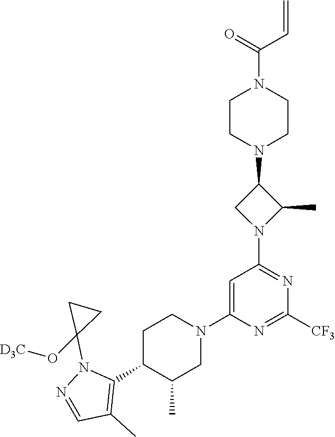 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)(F)F)n3)C[C@@H]2C)CC1] | 1-(4-((2R,3R)-1-(6- (4-(1-(1-(methoxy- (d3)cyclopropyl)-4- methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one | 2-397 | 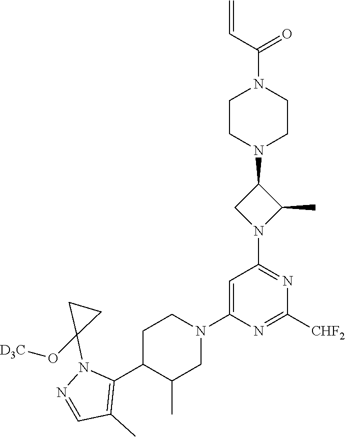 [SMILES: [2H]C([2H])([2H])OC1(n2ncc(C)c2C2CCN(c3cc(N4C[C@@H](N5CCN(C(=O)C=C)CC5)[C@H]4C)nc(C(F)F)n3)CC2C)CC1] | 1-(4-((2R,3R)-1-(2- (difluoromethyl)-6- (4-(1-(1-(methoxy- d3)cyclopropyl)-4- methyl-1H- pyrazol-5-yl)-3- methylpiperidin-1- yl)pyrimidin-4-yl)- 2-methylazetidin- 3-yl)piperazin-1- yl)prop-2-en-1-one |
| 2-395-1 | 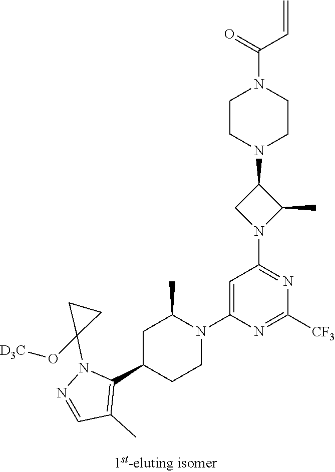 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)(F)F)n3)[C@H](C)C2)CC1] | 1-(4-((2R,3R)-1-(6- ((2R,4R)-4-(1-(1- (methoxy- d₃)cyclopropyl)-4- methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one | 2-397-1 | 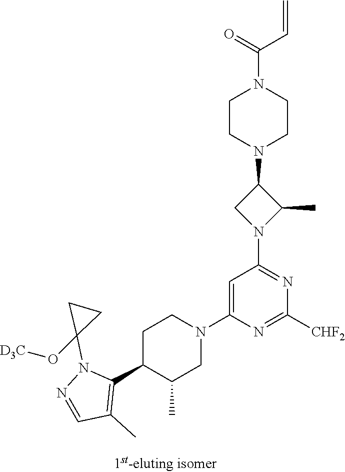 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)F)n3)C[C@@H]2C)CC1] | 1-(4-((2R,3R)-1-(2- (difluorometlyl)-6- ((3R,4S)-4-(1-(1- (methoxy- d₃)cyclopropyl)-4- methyl-1H- pyrazol-5-yl)-3- methylpiperidin-1- yl)pyrimidin-4-yl)- 2-methylazetidin- 3-yl)piperazin-1- yl)prop-2-en-1-one |
| 2-395-2 | 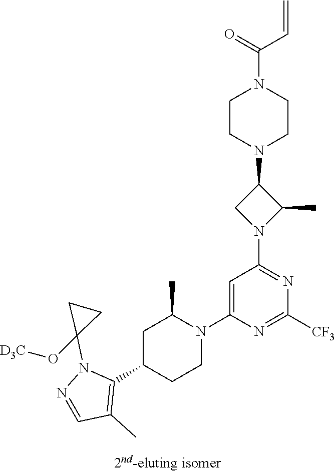 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)(F)F)n3)[C@H](C)C2)CC1] | 1-(4-((2R,3R)-1-(6- ((2R,4S)-4-(1-(1- (methoxy- d₃)cyclopropyl)-4- methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one | 2-397-2 | 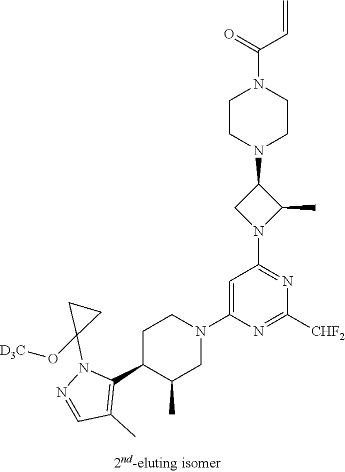 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)F)n3)C[C@H]2C)CC1] | 1-(4-((2R,3R)-1-(2- (difluoromethy])-6- ((3S,4S)-4-(1-(1- (methoxy- d₃)cyclopropyl)-4- methyl-1H- pyrazol-5-yl)-3- methylpiperidin-1- yl)pyrimidin-4-yl)- 2-methylazetidin- 3-yl)piperazin-1- yl)prop-2-en-1-one |
| 2-397-3 | 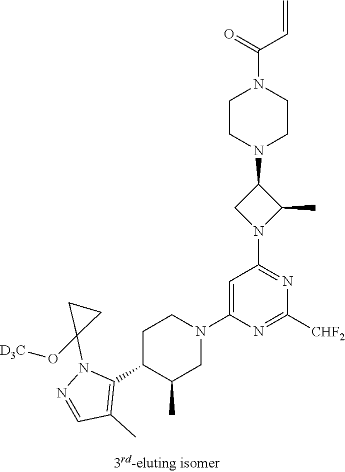 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)F)n3)C[C@H]2C)CC1] | 1-(4-((2R,3R)-1-(2- (difluoromethyl)-6- ((3S.4R)-4-(1-(1- (metboxy- d₃)cyclopropyl)-4- methyl-1H- pyrazol-5-yl)-3- methylpiperidin-1- yl)pyrimidin-4-yl)- 2-methylazetidin- 3-yl)piperazin-1- yl)prop-2-en-1-one | 2-400 | 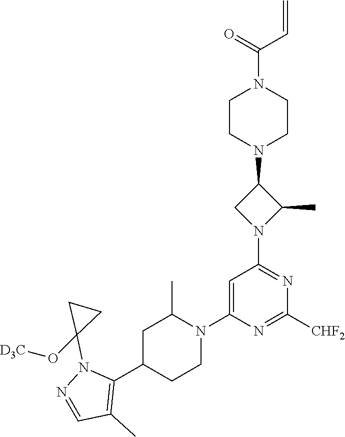 [SMILES: [2H]C([2H])([2H])OC1(n2ncc(C)c2C2CCN(c3cc(N4C[C@@H](N5CCN(C(=O)C=C)CC5)[C@H]4C)nc(C(F)F)n3)C(C)C2)CC1] | 1-(4-((2R.3R)-1-(2- (difluoromethyl)-6- (4-(1-(1-(methoxy- d3)cyclopropyl)-4- methyl-1H- pyrazol-5-yl)-2- methylpiperidin-1- yl)pyrimidin-4-yl)- 2-methylazetidin- 3-yl)piperazin-1- yl)prop-2-en-1-one |
| 2-397-4 | 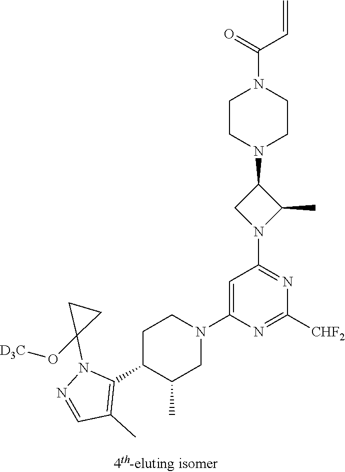 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)F)n3)C[C@@H]2C)CC1] | 1-(4-((2R,3R)-1-(2- (difluoromcthyl)-6- ((3R,4R)-4-(1-(1- (methoxy- d3)cyclopropyl)-4- methyl-1H- pyrazol-5-yl)-3- methylpiperidin-1- yl)pyrimidin-4-yl)- 2-methylazetidin- 3-yl)piperazin-1- yl)prop-2-en-1-one | 2-400-1 | 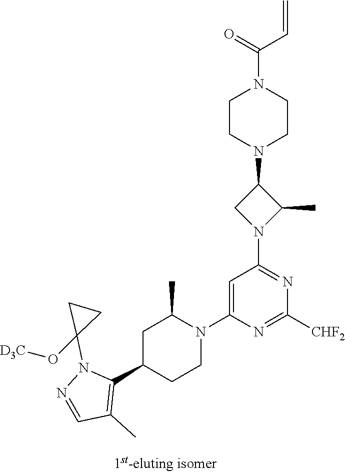 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)F)n3)[C@H](C)C2)CC1] | 1-(4-((2R,3R)-1-(2- (difluoromcthyl)-6- ((2R,4R)-4-(1-(1- (methoxy- d₂)cyclopropyl)-4- methyl-1H- pyrazol-5-yl)-2- methylpiperidin-1- yl)pyrimidin-4-yl)- 2-methylazetidin- 3-yl)piperazin-1- yl)prop-2-en-1-one |
| 2-398 | 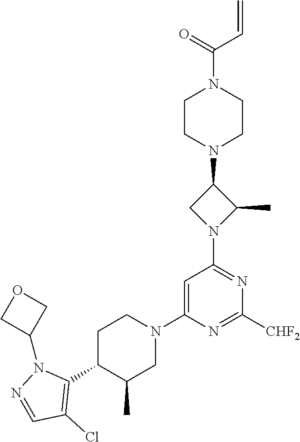 [SMILES: C=CC(=O)N1CCN([C@@H]2CN(c3cc(N4CC[C@@H](c5c(Cl)cnn5C5COC5)[C@H](C)C4)nc(C(F)F)n3)[C@@H]2C)CC1] | 1-(4-((2R,3R)-1-(6- ((3S,4R)-4-(4- chloro-1-(3- oxetanyl)-1H- pyrazol-5-yl)-3- methyl-1- piperidiny])-2- (difluoromethyl)-4- pyrimidinyl)-2- methyl-3- azetidinyl)-1- piperazinyl)-2- propen-1-one | 2-400-2 | 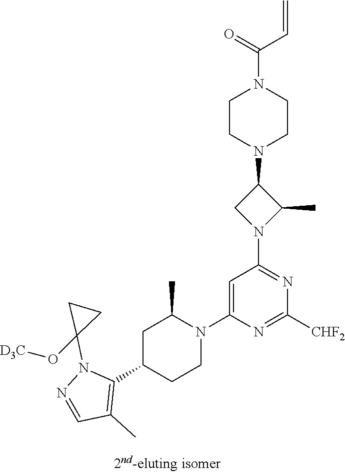 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)F)n3)[C@H](C)C2)CC1] | 1-(4-((2R,3R)-1-(2- (difluorometlyl)-6- ((2R,4S)-4-(1-(1- (methoxy- da)cyclopropyl)-4- methyl-1H- pyrazol-5-yl)-2- methylpiperidin-1- yl)pyrimidin-4-yl)- 2-methylazetidin- 3-yl)piperazin-1- yl)prop-2-en-1-one |
| 2-399 | 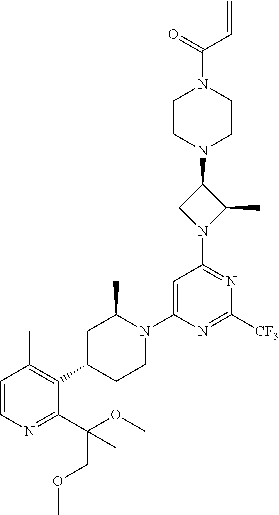 [SMILES: C=CC(=O)N1CCN([C@@H]2CN(c3cc(N4CC[C@H](c5c(C)ccnc5C(C)(COC)OC)C[C@H]4C)nc(C(F)(F)F)n3)[C@@H]2C)CC1] | 1-(4-((2R,3R)-1-(6- ((2R,4S)-4-(2-(1,2- dimethoxy-2- propanyl)-4- methyl-3- pyridinyl)-2- methyl-1- piperidiny])-2- (trifluoromethyl)- 1-pyrimidinyl)-2- methyl-3- azetidinyl)-1- piperazinyl)-2- propen-1-one | 2-401 | 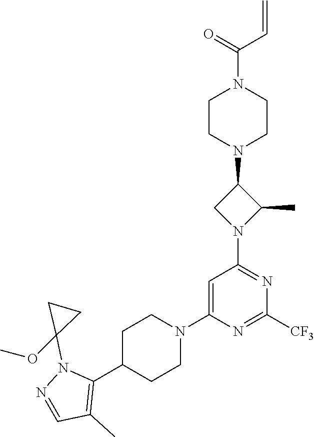 [SMILES: C=CC(=O)N1CCN([C@@H]2CN(c3cc(N4CCC(c5c(C)cnn5C5(OC)CC5)CC4)nc(C(F)(F)F)n3)[C@@H]2C)CC1] | 1-(4-((2R,3R)-1-(6- (4-(1-(1- methoxycyclo- propyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-402 | 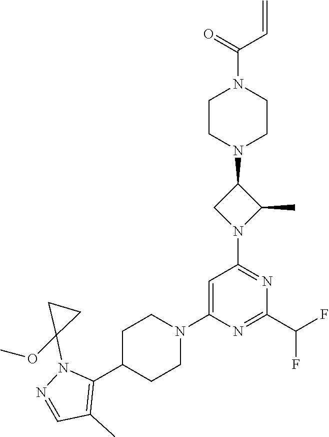 [SMILES: C=CC(=O)N1CCN([C@@H]2CN(c3cc(N4CCC(c5c(C)cnn5C5(OC)CC5)CC4)nc(C(F)F)n3)[C@@H]2C)CC1] | 1-(4-((2R,3R)-1-(2- (difluoromethyl)-6- (4-(1-(1- methoxycyclo- propyl)- 4-methyl-1H- pyrazol-5-yl)-1- piperidinyl)-4- pyrimidinyl)-2- methyl-3- azetidinyl)-1- piperazinyl)-2- propen-1-one | 2-406 | 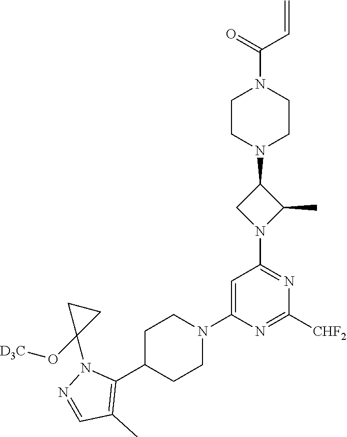 [SMILES: [2H]C([2H])([2H])OC1(n2ncc(C)c2C2CCN(c3cc(N4C[C@@H](N5CCN(C(=O)C=C)CC5)[C@H]4C)nc(C(F)F)n3)CC2)CC1] | 1-(4-((2R.3R)-1-(2- (difluoromethyl)-6- (4-(1-(1-(methoxy- d3)cyclopropyl)-4- methyl-1H- pyrazol-5- yl)piperidin-1- yl)pyrimidin-4-yl)- 2-methylazetidin- 3-yl)piperazin-1- yl)prop-2-en-1-one |
| 2-403 | 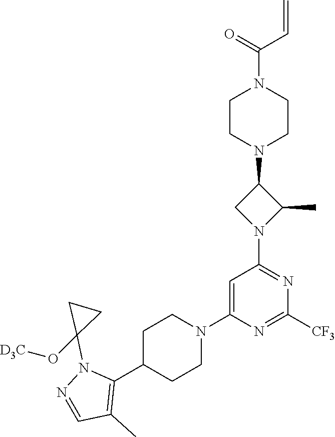 [SMILES: [2H]C([2H])([2H])OC1(n2ncc(C)c2C2CCN(c3cc(N4C[C@@H](N5CCN(C(=O)C=C)CC5)[C@H]4C)nc(C(F)(F)F)n3)CC2)CC1] | 1-(4-((2R,3R)-2- methyl-1-(6-(4-(1- (1-(methoxy- d3)cyclopropyl)-4- methyl-1H- pyrazol-5-yl)-1- piperidinyl)-2- (trifluoromethyl)- 4-pyrimidiny])-3- azetidinyl)-1- piperazinyl)-2- propen-1-one | 2-407 | 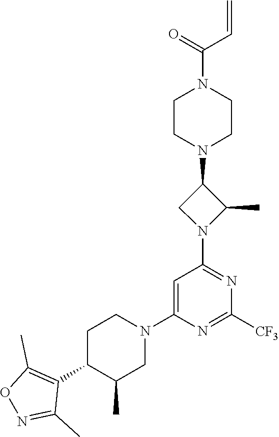 [SMILES: C=CC(=O)N1CCN([C@@H]2CN(c3cc(N4CC[C@@H](c5c(C)noc5C)[C@H](C)C4)nc(C(F)(F)F)n3)[C@@H]2C)CC1] | 1-(4-((2R,3R)-1-(6- ((3S,4R)-4-(3,5- dimethyl-1,2- oxazol-4-yl)-3- methyl-1- piperidiny])-2- (trifluoromethyl)- 4-pyrimidinyl)-2- methyl-3- azetidinyl)-1- piperazinyl)-2- propen-1-one |
| 2-404 | 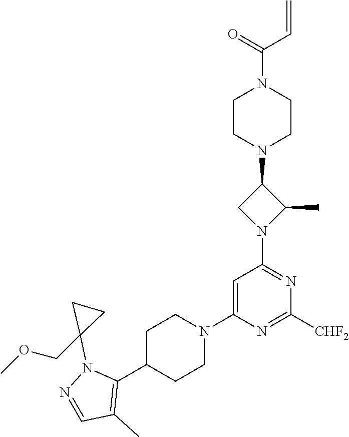 [SMILES: C=CC(=O)N1CCN([C@@H]2CN(c3cc(N4CCC(c5c(C)cnn5C5(COC)CC5)CC4)nc(C(F)F)n3)[C@@H]2C)CC1] | 1-(4-((2R,3R)-1-(2- (difluoromethyl)-6- (4-(1-(1- (methoxymethyl) cyclopropyl)-4- methyl-1H- pyrazol-5-yl)-1- piperidinyl)-4- pyrimidinyl)-2- methyl-3- azetidinyl)-1- piperazinyl)-2- propen-1-one | 2-408 | 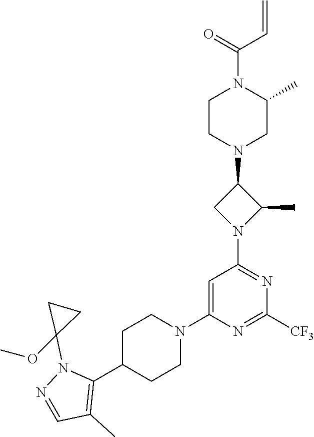 [SMILES: C=CC(=O)N1CCN([C@@H]2CN(c3cc(N4CCC(c5c(C)cnn5C5(OC)CC5)CC4)nc(C(F)(F)F)n3)[C@@H]2C)C[C@H]1C] | 1-((2R)-4- ((2R,3R)-1-(6-(4- (1-(1- methoxycyclo- propyl)-4- methyl-1H- pyrazol-5-yl)-1- piperidinyl)-2- (trifluoromethyl)- 4-pyrimidinyl)-2- methyl-3- azetidinyl)-2- mctbyl-1- piperazinyl)-2- propen-1-one |
| 2-405 | 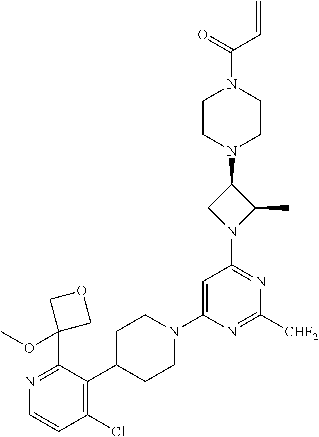 [SMILES: C=CC(=O)N1CCN([C@@H]2CN(c3cc(N4CCC(c5c(Cl)ccnc5C5(OC)COC5)CC4)nc(C(F)F)n3)[C@@H]2C)CC1] | 1-(4-((2R,3R)-1-(6- (4-(4-chloro-2-(3- methoxy-3- oxetanyl)-3- pyridinyl)-1- piperidinyl)-2- (difluoromethyl)-4- pyrimidinyl)-2- methyl-3- azetidiny])-1- piperazinyl)-2- propen-1-one | 2-409 | 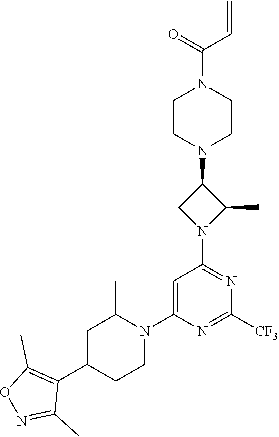 [SMILES: C=CC(=O)N1CCN([C@@H]2CN(c3cc(N4CCC(c5c(C)noc5C)CC4C)nc(C(F)(F)F)n3)[C@@H]2C)CC1] | 1-(4-((2R,3R)-1-(6- (4-(3.5-dimethyl- 1,2-oxazol-4-yl)-2- methyl-1- pipcridinyl)-2- (trifluoromethyl)- 4-pyrimidinyl)-2- methyl-3- azetidinyl)-1- piperazinyl)-2- propen-t-one |
| 2-409-1 | 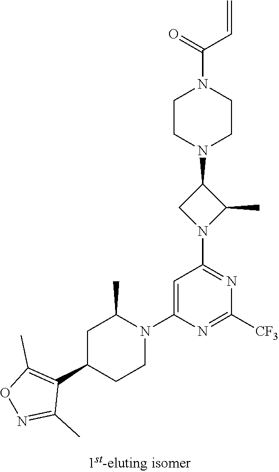 [SMILES: C=CC(=O)N1CCN([C@@H]2CN(c3cc(N4CC[C@@H](c5c(C)noc5C)C[C@H]4C)nc(C(F)(F)F)n3)[C@@H]2C)CC1] | 1-(4-((2R,3R)-1-(6- ((2R,4R)-4-(3,5- dimethyl-1,2- oxazol-4-y])-2- methyl-1- piperidinyl)-2- (trifluoromethyl)- 4-pyrimidiny])-2- methyl-3- azetidinyl)-1- piperazinyl)-2- propen-1-one | 2-412 | 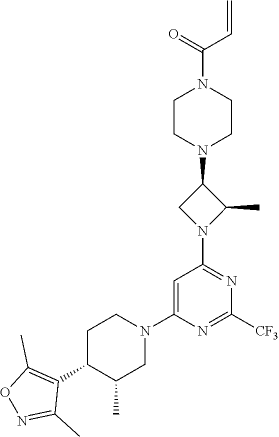 [SMILES: C=CC(=O)N1CCN([C@@H]2CN(c3cc(N4CC[C@@H](c5c(C)noc5C)[C@@H](C)C4)nc(C(F)(F)F)n3)[C@@H]2C)CC1] | 1-(4-((2R,3R)-1-(6- ((3R,4R)-4-(3.5- dimethyl-1,2- oxazol-4-yl)-3- methyl-1- piperidinyl)-2- (trifluoromethyl)- 4-pyrimidinyl)-2- methyl-3- azetidinyl)-1- piperazinyl)-2- propen-1-one |
| 2-409-2 | 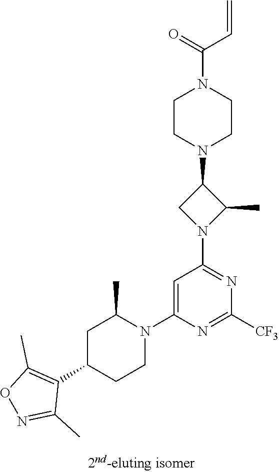 [SMILES: C=CC(=O)N1CCN([C@@H]2CN(c3cc(N4CC[C@H](c5c(C)noc5C)C[C@H]4C)nc(C(F)(F)F)n3)[C@@H]2C)CC1] | 1-(4-((2R,3R)-1-(6- ((2R,4S)-4-(3,5- dimethyl-1,2- oxazol-4-yl)-2- methyl-1- piperidiny])-2- (trifluoromethyl)- 4-pyrimidinyl)-2- methyl-3- azetidinyl)-1- piperazinyl)-2- propen-1-one | 2-413 | 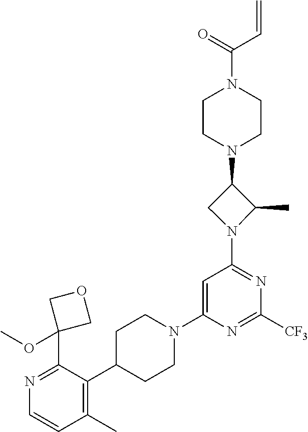 [SMILES: C=CC(=O)N1CCN([C@@H]2CN(c3cc(N4CCC(c5c(C)ccnc5C5(OC)COC5)CC4)nc(C(F)(F)F)n3)[C@@H]2C)CC1] | 1-(4-((2R,3R)-1-(6- (4-(2-(1- methoxycyclo- propyl)-4-methyl- 3-pyridinyl)-1- piperidinyl)-2- (trifluoromethyl)- 4-pyrimidinyl)-2- methyl-3- azetidinyl)-1- piperazinyl)-2- propen-1-one |
| 2-410 | 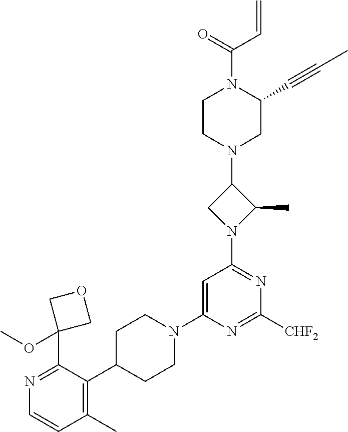 [SMILES: C=CC(=O)N1CCN(C2CN(c3cc(N4CCC(c5c(C)ccnc5C5(OC)COC5)CC4)nc(C(F)F)n3)[C@@H]2C)C[C@H]1C#CC] | 1-((2R)-4- ((2R,3R)-1-(2- (difluoromethyl)-6- (4-(2-(3-methoxy- 3-oxetanyl)-4- methyl-3- pyridinyl)-1- piperidinyl)-4- pyrimidinyl)-2- methyl-3- azetidinyl)-2-(1- propyn-1-yl)-1- piperazinyl)-2- propen-1-one | 2-414 | 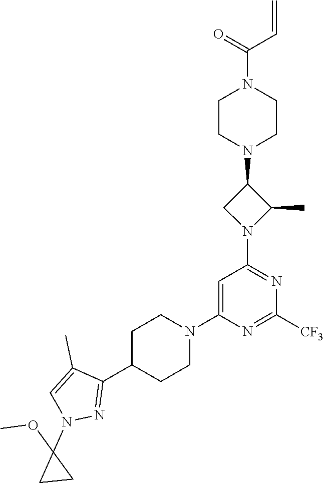 [SMILES: C=CC(=O)N1CCN([C@@H]2CN(c3cc(N4CCC(c5nn(C6(OC)CC6)cc5C)CC4)nc(C(F)(F)F)n3)[C@@H]2C)CC1] | 1-(4-((2R,3R)-1-(6- (4-(1-(1- methoxycyclo- propyl)-4- methyl-1H- pyrazol-3-yl)-1- piperidinyl)-2- (trifluoromethyl)- 4-pyrimidinyl)-2- methyl-3- azetidinyl)-1- piperazinyl)-2- propen-1-one |
| 2-411 | 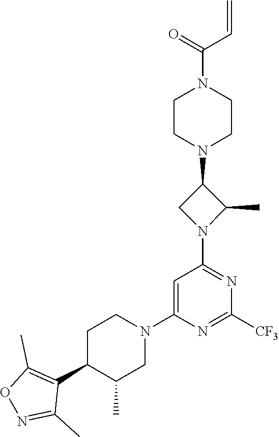 [SMILES: C=CC(=O)N1CCN([C@@H]2CN(c3cc(N4CC[C@H](c5c(C)noc5C)[C@@H](C)C4)nc(C(F)(F)F)n3)[C@@H]2C)CC1] | 1-(4-((2R,3R)-1-(6- ((3R,4S)-4-(3.5- dimethyl-1,2- oxazol-4-yl)-3- methyl-1- piperidinyl)-2- (trifluoromethyl)- 4-pyrimidinyl)-2- methyl-3- azetidiny])-1- piperazinyl)-2- propen-1-one | 2-415 | 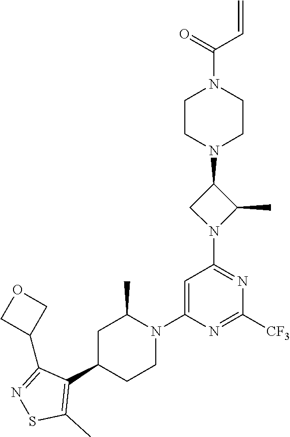 [SMILES: C=CC(=O)N1CCN([C@@H]2CN(c3cc(N4CC[C@@H](c5c(C6COC6)nsc5C)C[C@H]4C)nc(C(F)(F)F)n3)[C@@H]2C)CC1] | 1-(4-((2R,3R)-2- methyl-1-(6- ((2R,4R)-2-methyl- 4-(5-methyl-3-(3- oxetanyl)-1,2- thiazol-4-yl)-1- piperidinyl)-2- (trifluoromethyl)- 4-pyrimidinyl)-3- azetidinyl)-1- piperaziny])-2- propen-1-one |
| 2-416 | 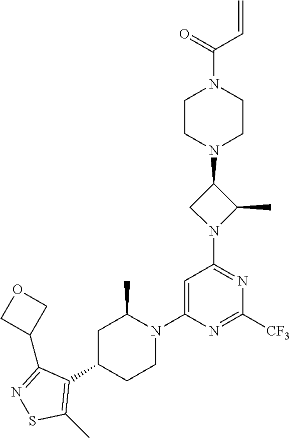 [SMILES: C=CC(=O)N1CCN([C@@H]2CN(c3cc(N4CC[C@H](c5c(C6COC6)nsc5C)C[C@H]4C)nc(C(F)(F)F)n3)[C@@H]2C)CC1] | 1-(4-((2R,3R)-2- methyl-1-(6- ((2R,4S)-2-methyl- 4-(5-methyl-3-(3- oxetanyl)-1,2- thiazol-4-yl)-1- piperidinyl)-2- (trifluoromethyl)- 4-pyrimidinyl)-3- azetidinyl)-1- piperazinyl)-2- propen-1-one | 2-420 | 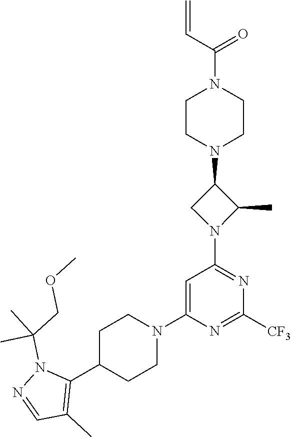 [SMILES: C=CC(=O)N1CCN([C@@H]2CN(c3cc(N4CCC(c5c(C)cnn5C(C)(C)COC)CC4)nc(C(F)(F)F)n3)[C@@H]2C)CC1] | 1-(4-((2R,3R)-1-(6- (4-(1-(1-methoxy- 2-methyl-2- propanyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-417 | 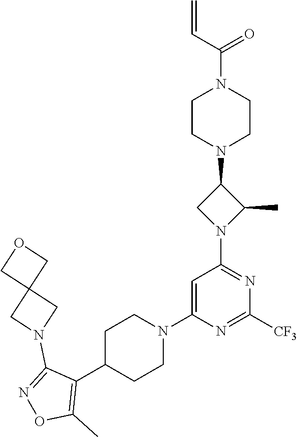 [SMILES: C=CC(=O)N1CCN([C@@H]2CN(c3cc(N4CCC(c5c(N6CC7(COC7)C6)noc5C)CC4)nc(C(F)(F)F)n3)[C@@H]2C)CC1] | 1-(4-((2R,3R)-2- methyl-1-(6-(4-(5- methyl-3-(2-oxa-6- azaspiro[3.3]heptan- 6-yl)-1,2-oxazol- 4-yl)-1- piperidinyl-2- (trifluoromethyl)- 4-pyrimidiny])-3- azetidinyl)-1- piperazinyl)-2- propen-l-one | 2-421 | 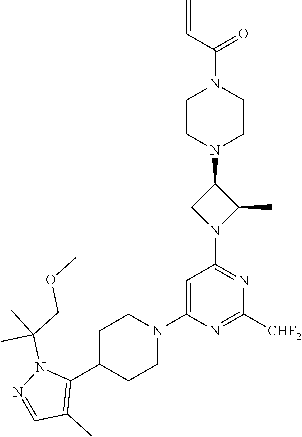 [SMILES: C=CC(=O)N1CCN([C@@H]2CN(c3cc(N4CCC(c5c(C)cnn5C(C)(C)COC)CC4)nc(C(F)F)n3)[C@@H]2C)CC1] | 1-(4-((2R,3R)-1-(2- (difluoromethyl)-6- (4-(1-(1-methoxy- 2-methyl-2- propanyl)-4- methyl-1H- pyrazol-5-yl)-1- piperidinyl)-4- pyrimidinyl)-2- methyl-3- azetidinyl)-]- piperazinyl)-2- propen-1-one |
| 2-418 | 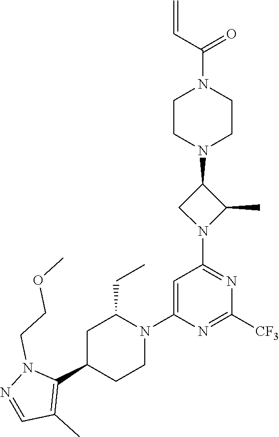 [SMILES: C=CC(=O)N1CCN([C@@H]2CN(c3cc(N4CC[C@@H](c5c(C)cnn5CCOC)C[C@@H]4CC)nc(C(F)(F)F)n3)[C@@H]2C)CC1] | 1-(4-((2R,3R)-1-(6- ((2S,4R)-2-ethyl-4- (1-(2- methoxyethyl)-4- methyl-1H- pyrazol-5-yl)-1- piperidiny]l)-2- (trifluoromethyl)- 4-pyrimidinyl)-2- methyl-3- azetidinyl)-1- piperazinyl)-2- propen-1-one | 2-422 | 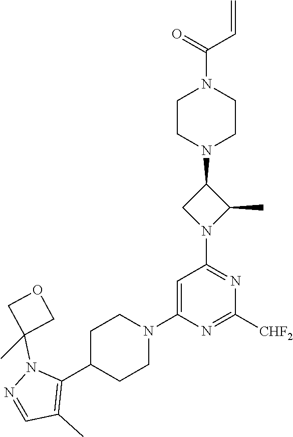 [SMILES: C=CC(=O)N1CCN([C@@H]2CN(c3cc(N4CCC(c5c(C)cnn5C5(C)COC5)CC4)nc(C(F)F)n3)[C@@H]2C)CC1] | 1-(4-((2R,3R)-1-(2- (difluorometliy])-6- (4-(4-methyl-1-(3- methyl-3- oxetanyl)-1H- pyrazol-5-yl)-1- piperidinyl)-4- pyrimidinyl)-2- methyl-3- azetidinyl)-1- piperazinyl)-2- propen-1-one |
| 2-419 | 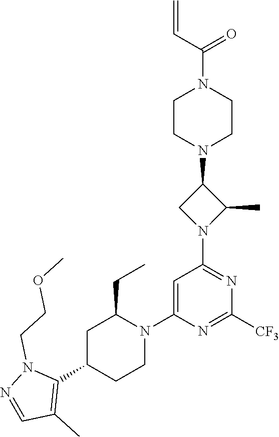 [SMILES: C=CC(=O)N1CCN([C@@H]2CN(c3cc(N4CC[C@H](c5c(C)cnn5CCOC)C[C@H]4CC)nc(C(F)(F)F)n3)[C@@H]2C)CC1] | 1-(4-((2R,3R)-1-(6- ((2R,4S)-2-ethyl-4- (1-(2- methoxyethyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-423 | 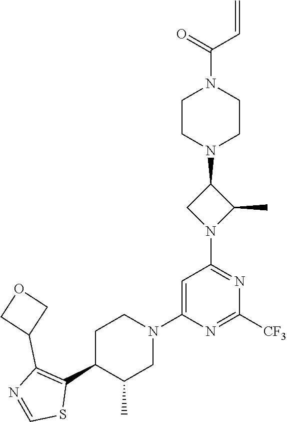 [SMILES: C=CC(=O)N1CCN([C@@H]2CN(c3cc(N4CC[C@H](c5scnc5C5COC5)[C@@H](C)C4)nc(C(F)(F)F)n3)[C@@H]2C)CC1] | 1-(4-((2R,3R)-2- methyl-1-(6- ((3R.4S)-3-methyl- 4-(4-(3-oxetanyl)- 1,3-thiazol-5-yl)-1- piperidinyl)-2- (trifluoromethyl)- 4-pyrimidinyl)-3- azetidinyl)-1- piperazinyl)-2- propen-t-one |
| 2-424 | 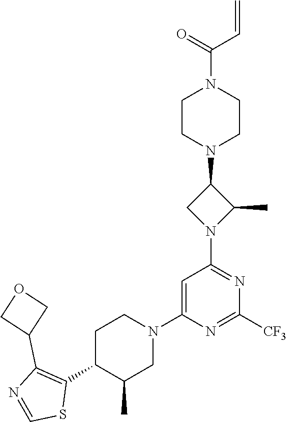 [SMILES: C=CC(=O)N1CCN([C@@H]2CN(c3cc(N4CC[C@@H](c5scnc5C5COC5)[C@H](C)C4)nc(C(F)(F)F)n3)[C@@H]2C)CC1] | 1-(4-((2R,3R)-2- methyl-1-(6- ((3S,4R)-3-methyl- 4-(4-(3-oxetanyl)- 1,3-thiazol-5-yl)-1- piperidinyl)-2- (trifluoromethyl)- 4-pyrimidinyl)-3- azetidinyl)-1- piperazinyl)-2- propen-1-one | 2-427 | 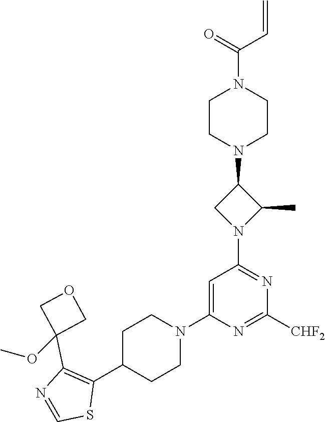 [SMILES: C=CC(=O)N1CCN([C@@H]2CN(c3cc(N4CCC(c5scnc5C5(OC)COC5)CC4)nc(C(F)F)n3)[C@@H]2C)CC1] | 1-(4-((2R.3R)-1-(2- (difluoromethyl)-6- (4-(4-(3-methoxy- 3-oxetanyl)-1,3- thiazol-5-yl)-1- piperidinyl)-4- pyrimidinyl)-2- methyl-3- azetidinyl)-1- piperazinyl)-2- propen-1-one |
| 2-425 | 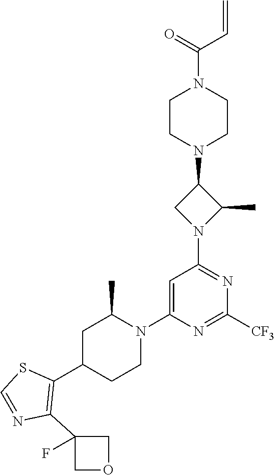 [SMILES: C=CC(=O)N1CCN([C@@H]2CN(c3cc(N4CCC(c5scnc5C5(F)COC5)C[C@H]4C)nc(C(F)(F)F)n3)[C@@H]2C)CC1] | 1-(4-((2R,3R)-1-(6- ((2R)-4-(4-(3- fhiorooxetan-3- yl)thiazol-5-yl)-2- methylpiperidin-1- yl)-2- (trifluoromethyl) pyrimidin-4-yl)-2- methylazetidin-3- yl)piperazin-1- yl)prop-2-en-1-one | 2-428 | 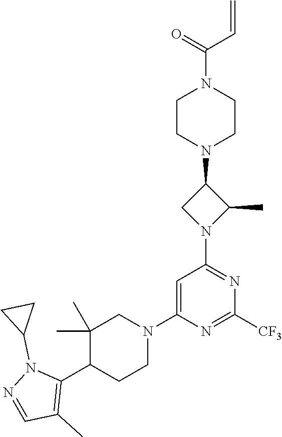 [SMILES: C=CC(=O)N1CCN([C@@H]2CN(c3cc(N4CCC(c5c(C)cnn5C5CC5)C(C)(C)C4)nc(C(F)(F)F)n3)[C@@H]2C)CC1] | 1-(4-((2R,3R)-1-(6- (4-(1-cyclopropyl- 4-methyl-1H- pyrazol-5-yl)-3,3- dimethylpiperidin- 1-yl)-2- (trifluoromethyl) pyrimidin-4-yl)-2- methylazetidin-3- yl)piperazin-1- yl)prop-2-en-1-one |
| 2-425-1 | 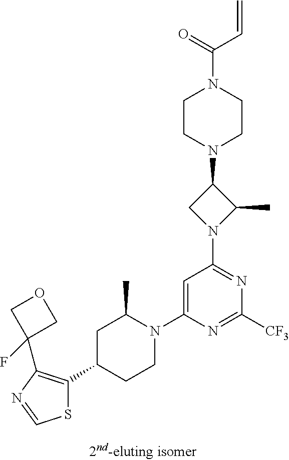 [SMILES: C=CC(=O)N1CCN([C@@H]2CN(c3cc(N4CC[C@H](c5scnc5C5(F)COC5)C[C@H]4C)nc(C(F)(F)F)n3)[C@@H]2C)CC1] | 1-(4-((2R,3R)-1-(6- ((2R,4R)-4-(4-(3- fluoro-3-oxetanyl)- 1,3-thiazol-5-yl)-2- methyl-1- piperidinyl)-2- (trifluoromethyl)- 4-pyrimidinyl)-2- methyl-3- azetidinyl)-1- piperazinyl)-2- propen-1-one | 2-428-1 | 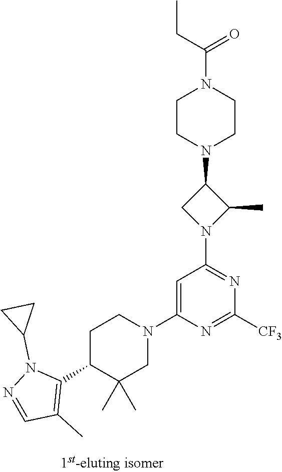 [SMILES: CCC(=O)N1CCN([C@@H]2CN(c3cc(N4CC[C@@H](c5c(C)cnn5C5CC5)C(C)(C)C4)nc(C(F)(F)F)n3)[C@@H]2C)CC1] | 1-(4-((2R,3R)-1-(6- ((4R)-4-(1- cyclopropyl-4- methyl-1H- pyrazol-5-yl)-3,3- dimethyl-1- piperidiny])-2- (trifluoromethyl)- 4-pyrimidinyl)-2- methyl-3- azetidinyl)-1- piperazinyl)-2- propen-1-one |
| 2-426 | 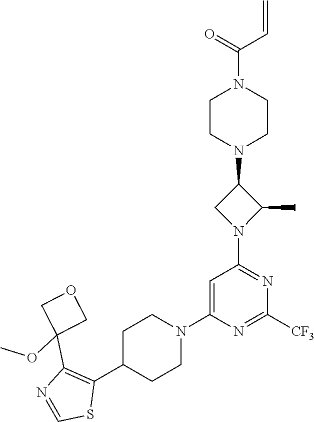 [SMILES: C=CC(=O)N1CCN([C@@H]2CN(c3cc(N4CCC(c5scnc5C5(OC)COC5)CC4)nc(C(F)(F)F)n3)[C@@H]2C)CC1] | 1-(4-((2R,3R)-1-(6- (4-(4-(3- methoxyoxetan-3- ylthiazol-5- yl)piperidin-1-yl)- 2-(trifluoromethyl) pyrimidin-4-yl)-2- methylazetidin-3- yl)piperazin-1- yl)prop-2-en-1-one | 2-428-2 | 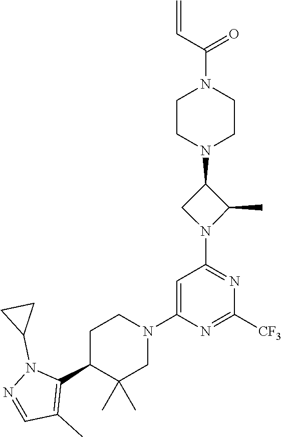 [SMILES: C=CC(=O)N1CCN([C@@H]2CN(c3cc(N4CC[C@H](c5c(C)cnn5C5CC5)C(C)(C)C4)nc(C(F)(F)F)n3)[C@@H]2C)CC1] | 1-(4-((2R,3R)-1-(6- ((4S)-4-(1- cyclopropyl-4- methyl-1H- pyrazol-5-yl)-3,3- dimethyl-1- piperidinyl)-2- (trifluoromethyl)- 4-pyrimidinyl)-2- methyl-3- azetidinyl)-1- piperaziny])-2- propen-1-one |
| | | | | 2nd-eluting isomer | |
| 2-429 | 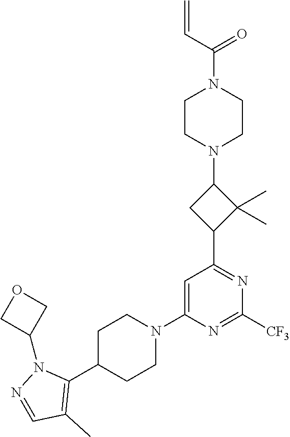 [SMILES: C=CC(=O)N1CCN(C2CC(c3cc(N4CCC(c5c(C)cnn5C5COC5)CC4)nc(C(F)(F)F)n3)C2(C)C)CC1] | 1-(4-(2,2-dimethy]- 3-(6-(4-(4-methyl- 1-(3-oxetanyl)-1H- pyrazol-5-yl)-1- piperidiny])-2- (trifluoromethyl)- 4-pyrimidinyl) cyclobutyl)-1- piperazinyl)-2- propen-1-one | 2-433 | 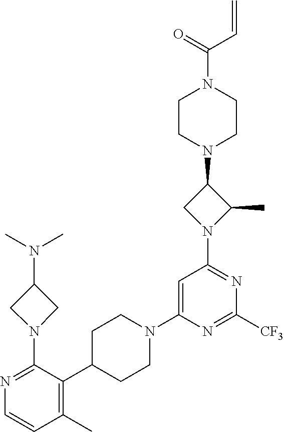 [SMILES: C=CC(=O)N1CCN([C@@H]2CN(c3cc(N4CCC(c5c(C)ccnc5N5CC(N(C)C)C5)CC4)nc(C(F)(F)F)n3)[C@@H]2C)CC1] | 1-(4-((2R,3R)-1-(6- (4-(2-(3- (dimethylamino)-1- azetidinyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| | mixture of cis isomers | | | | |
| 2-430 | 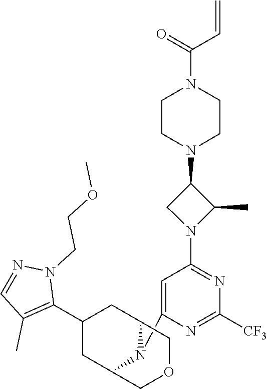 [SMILES: C=CC(=O)N1CCN([C@@H]2CN(c3cc(N4[C@@H]5COC[C@H]4CC(c4c(C)cnn4CCOC)C5)nc(C(F)(F)F)n3)[C@@H]2C)CC1] | 1-(4-((2R,3R)-1-(6- ((7-endo)-7-(1-(2- methoxycthyl)-4- methyl-1H- pyrazol-5-yl)-3- oxa-9- azabicyclo[3.3.1] nonan-9-yl)-2- (trifluoromethyl)- 4-pyrimidinyl)-2- methyl-3- azetidinyl)-1- piperazinyl)-2- propen-1-one | 2-434 | 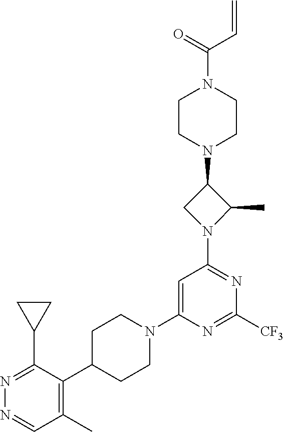 [SMILES: C=CC(=O)N1CCN([C@@H]2CN(c3cc(N4CCC(c5c(C)cnnc5C5CC5)CC4)nc(C(F)(F)F)n3)[C@@H]2C)CC1] | 1-(4-((2R,3R)-1-(6- (4-(3-cyclopropyl- 5-methyl-4- pyridaziny])-1- piperidinyl)-2- (trifluoromethyl)- 4-pyrimidinyl)-2- methyl-3- azetidinyl)-1- piperazinyl)-2- propen-1-one |
| 2-431 | 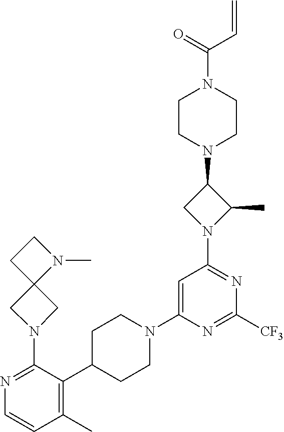 [SMILES: C=CC(=O)N1CCN([C@@H]2CN(c3cc(N4CCC(c5c(C)ccnc5N5CC6(CCN6C)C5)CC4)nc(C(F)(F)F)n3)[C@@H]2C)CC1] | 1-(4-((2R,3R)-2- methyl-1-(6-(4-(4- methyl-2-(1- methyl-1.6- diazaspiro[3.3] heptan-6-yl)-3- pyridinyl)-1- piperidinyl)-2- (trifluoromethyl)- 4-pyrimidinyl)-3- azetidinyl)-1- piperazinyl)-2- propen-1-one | 2-435 | 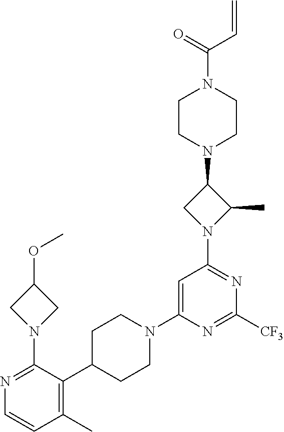 [SMILES: C=CC(=O)N1CCN([C@@H]2CN(c3cc(N4CCC(c5c(C)ccnc5N5CC(OC)C5)CC4)nc(C(F)(F)F)n3)[C@@H]2C)CC1] | 1-(4-((2R,3R)-1-(6- (4-(2-(3-methoxy- 1-azetidinyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-432 | 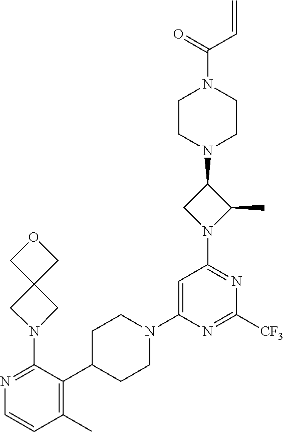 [SMILES: C=CC(=O)N1CCN([C@@H]2CN(c3cc(N4CCC(c5c(C)ccnc5N5CC6(COC6)C5)CC4)nc(C(F)(F)F)n3)[C@@H]2C)CC1] | 1-(4-((2R,3R)-2- methyl-1-(6-(4-(4- methyl-2-(2-oxa-6- azaspiro[3.3]heptan- 6-yl)-3-pyridiuyl)- I-piperidinyl)-2- (trifluoromethyl)- 4-pyrimidinyl)-3- azetidinyl)-1- piperazinyl)-2- propen-1-one | 2-436 | 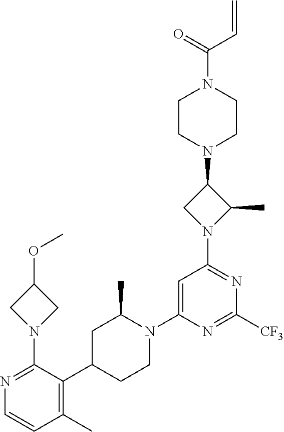 [SMILES: C=CC(=O)N1CCN([C@@H]2CN(c3cc(N4CCC(c5c(C)ccnc5N5CC(OC)C5)C[C@H]4C)nc(C(F)(F)F)n3)[C@@H]2C)CC1] | 1-(4-((2R,3R)-1-(6- ((2R)-4-(2-(3- methoxy-1- azetidinyl)-4- methyl-3- pyridinyl)-2- methyl-1- piperidiny])-2- (trifluoromethyl)- 4-pyrimidinyl)-2- methyl-3- azetidinyl)-1- piperazinyl)-2- propen-1-one |
| | | | | mixture of cis/trans isomers | |
| 2-437 | 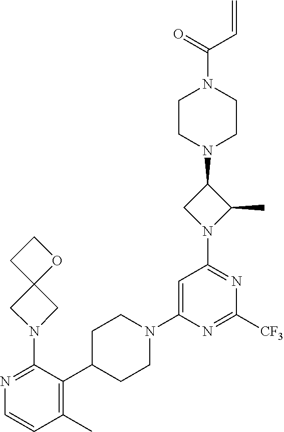 [SMILES: C=CC(=O)N1CCN([C@@H]2CN(c3cc(N4CCC(c5c(C)ccnc5N5CC6(CCO6)C5)CC4)nc(C(F)(F)F)n3)[C@@H]2C)CC1] | 1-(4-((2R,3R)-2- methyl-1-16-(4-(4- methyl-2-(1-oxa-6- azaspiro[3.3]heptan- 6-yl)-3-pyridinyl)- 1-piperidinyl)-2- (trifluoromcthyl)- 4-pyrimidinyl)-3- azetidinyl)-1- piperazinyl)-2- propen-1-one | 2-440 | 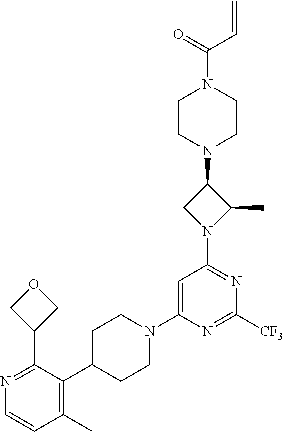 [SMILES: C=CC(=O)N1CCN([C@@H]2CN(c3cc(N4CCC(c5c(C)ccnc5C5COC5)CC4)nc(C(F)(F)F)n3)[C@@H]2C)CC1] | 1-(4-((2R,3R)-2- methyl-1-(6-(4-(4- methyl-2-(3- oxetanyl)-3- pyridinyl)-1- piperidinyl)-2- (trifluoromethyl)- 4-pyrimidinyl)-3- azetidinyl)-1- piperazinyl)-2- propen-1-one |
| 2-438 | 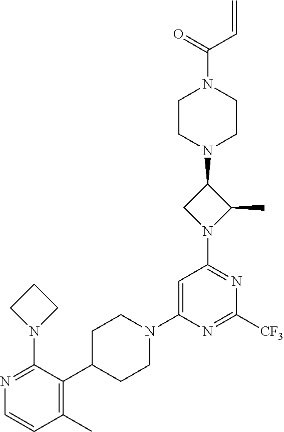 [SMILES: C=CC(=O)N1CCN([C@@H]2CN(c3cc(N4CCC(c5c(C)ccnc5N5CCC5)CC4)nc(C(F)(F)F)n3)[C@@H]2C)CC1] | 1-(4-((2R,3R)-1-(6- (4-(2-(azetidin-1- yl)-4- methylpyridin-3- yl)piperidin-1-yl)- 2-(trifluoromethyl) pyrimidin-4-yl)-2- methylazetidin-3- yl)piperazin-1- yl)prop-2-en-1-one | 2-441 | 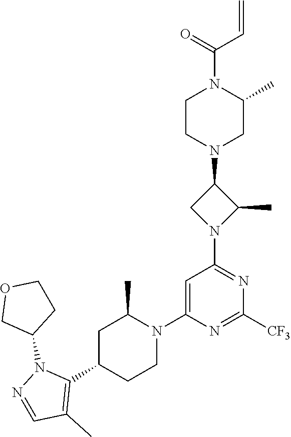 [SMILES: C=CC(=O)N1CCN([C@@H]2CN(c3cc(N4CC[C@H](c5c(C)cnn5[C@H]5CCOC5)C[C@H]4C)nc(C(F)(F)F)n3)[C@@H]2C)C[C@H]1C] | 1-((2R)-2-methyl- 4-((2R,3R)-2- methyl-1-(6- ((2R,45)-2-methyl- 4-(4-methyl-1- ((3S)-tetrahydro-3- furanyl)-1H- pyrazol-5-yl)-1- piperidinyl)-2- (trifloromethyl)- 4-pyrimidinyl)-3- azetidinyl)-1- piperazinyl)-2- propen-1-one |
| 2-439 | 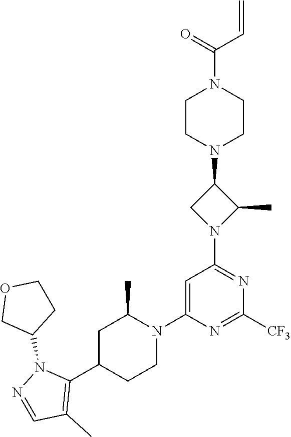 | 1-(4-((2R,3R)-2- methyl-1-(6- ((2R,4S)-2-methyl- 4-(4-methyl-1-((S)- tetrahydrofuran-3- yl)-1H-pyrazol-5- yl)piperidin-1-yl)- 2.(trifluoromethyl) pyrimidin-1- ylazetidin-3- yl)piperazin-1- [symbol] | 2-442 | 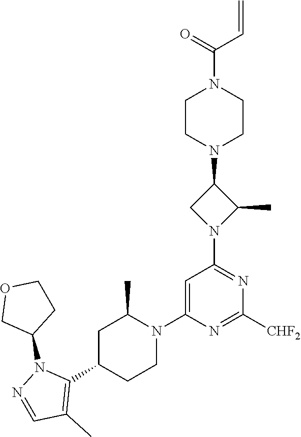 [SMILES: C=CC(=O)N1CCN([C@@H]2CN(c3cc(N4CC[C@H](c5c(C)cnn5[C@@H]5CCOC5)C[C@H]4C)nc(C(F)F)n3)[C@@H]2C)CC1] | 1-(4-((2R,3R)-1-(2- (difluoromethyl)-6- ((2R,4S)-2-methyl- 4-(4-methyl-1- ((3R)-tetrahydro-3- furanyl)-1H- pyrazol-5-yl)-1- piperidinyl)-4- pyrimidinyl)-2- methyl-3- azetidinyl)-1- piperazinyl)-2- propen-1-one |
| 2-439-1 | 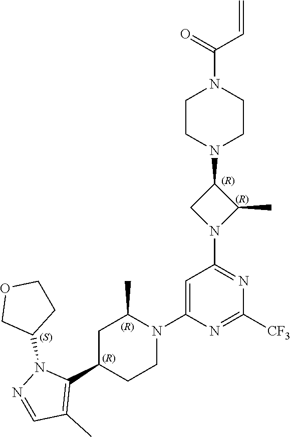 | 1-(4-((2R,3R)-2- methyl-1-(6- ((2R,4R)-2-methyl- 4-(4-methyl-1-((S)- tetrahydrofuran-3- yl)-1H-pyrazol-5- yl)piperidin-1-yl)- 2- (trifluoromethyl) pyrimidin-4- ylazetidin-3- yl)piperazin-1- [symbol] | 2-443 | 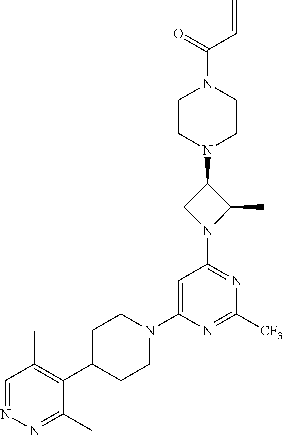 [SMILES: C=CC(=O)N1CCN([C@@H]2CN(c3cc(N4CCC(c5c(C)cnnc5C)CC4)nc(C(F)(F)F)n3)[C@@H]2C)CC1] | 1-(4-((2R,3R)-1-(6- (4-(3,5-dimethyl-4- pyridazinyl)-1- piperidinyl)-2- (trifluoromethyl)- 4-pyrimidinyl)-2- methyl-3- azetidinyl)-1- piperazinyl)-2- propen-1-one |
| | 1st-eluting isomer | | | | |
| 2-444 | 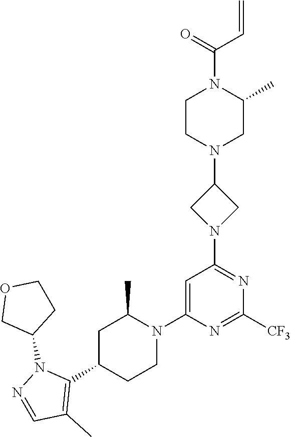 [SMILES: C=CC(=O)N1CCN(C2CN(c3cc(N4CC[C@H](c5c(C)cnn5[C@H]5CCOC5)C[C@H]4C)nc(C(F)(F)F)n3)C2)C[C@H]1C] | 1-((2R)-2-methyl- 4-(1-(6-((2R,4S)-2- methyl-4-(4- methyl-1-((35)- tetrabydro-3- furanyl)-1H- pyrazol-5-yl)-1- piperidinyl)-2- (trifluoromethyl)- 4-pynimidinyl)-3- azetidinyl)-1- piperazinyl)-2- propen-1-one | 2-448 | 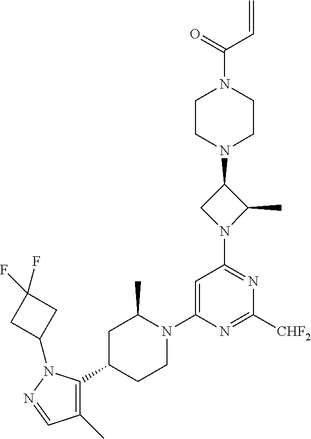 [SMILES: C=CC(=O)N1CCN([C@@H]2CN(c3cc(N4CC[C@H](c5c(C)cnn5C5CC(F)(F)C5)C[C@H]4C)nc(C(F)F)n3)[C@@H]2C)CC1] | 1-(4-((2R,3R)-1-(6- ((2R,4S)-4-(1-(3,3- difluorocyclo- butyl)-4-methyl- 1H-pyrazol-5-yl)- 2-methyl-1- piperidinyl)-2- (difluoromcthyl)-4- pyrimidinyl)-2- methyl-3- azetidinyl)-1- piperazinyl)-2- propen-1-one |
| 2-445 | 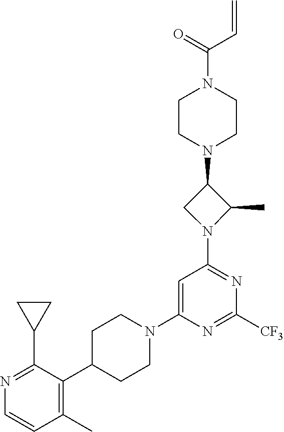 [SMILES: C=CC(=O)N1CCN([C@@H]2CN(c3cc(N4CCC(c5c(C)ccnc5C5CC5)CC4)nc(C(F)(F)F)n3)[C@@H]2C)CC1] | 1-(4-((2R,3R)-1-(6- (4-(2-cyclopropyl- 4-methyl-3- pyridinyl)-1- piperidinyl)-2- (trifluoromethyl)- 4-pyrimidinyl)-2- methyl-3- azetidinyl)-1- piperazinyl)-2- propen-1-one | 2-449 | 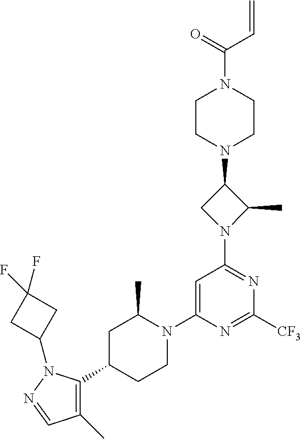 [SMILES: C=CC(=O)N1CCN([C@@H]2CN(c3cc(N4CC[C@H](c5c(C)cnn5C5CC(F)(F)C5)C[C@H]4C)nc(C(F)(F)F)n3)[C@@H]2C)CC1] | 1-(4-((2R,3R)-1-(6- ((2R,4S)-4-(1-(3,3- difluorocyclo- butyl)-4-methyl-1H- pyrazol-5-yl)-2- methyl-1- piperidinyl)-2- (trifluoromethyl)- 4-pyrimidinyl)-2- methyl-3- azetidinyl)-1- piperazinyl)-2- propen-1-one |
| 2-446 | 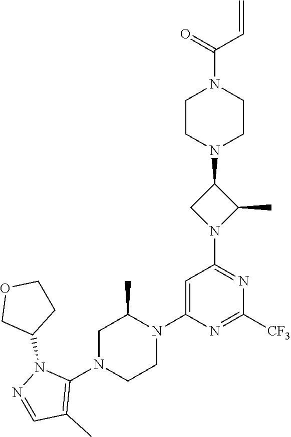 | 1-(4-((2R,3R)-2- methyl-1-(6-((R)- 2-methyl-4-(4- methyl-1-((S)- tetrahydrofuran-3- yl)-1H-pyrazol-5- yl)piperazin-1-yl)- 2- (trifluoromethyl) pyrimidin-4- [symbol] | 2-450 | 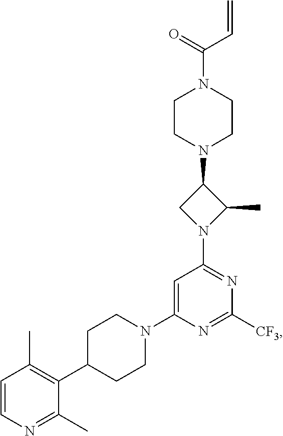 [SMILES: C=CC(=O)N1CCN([C@@H]2CN(c3cc(N4CCC(c5c(C)ccnc5C)CC4)nc(C(F)(F)F)n3)[C@@H]2C)CC1] | 1-(4-((2R,3R)-1-(6- (4-(2,4-dimethyl-3- pyridinyl)-1- piperidinyl)-2- (trifluoromethyl)- 4-pyrimidinyl)-2- methyl-3- azetidinyl)-1- piperazinyl)-2- propon-t-one |
| 2-447 | 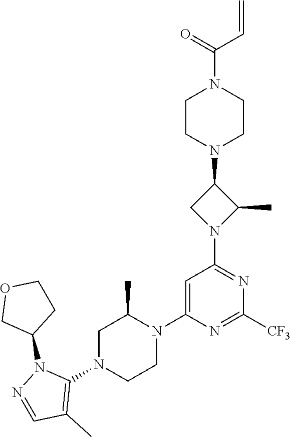 | 1-(4-((2R,3R)-1-(2- (difluoromethyl)-6- ((2R,4S)-2-methyl- 4-(4-methyl-1- ((R)- tetrahydrofuran-3- yl)-1H-pyrazol-5- yl)piperidin-1- yl)pyrimidin-4-yl)- 2-methylazetidin- | 2-451 | 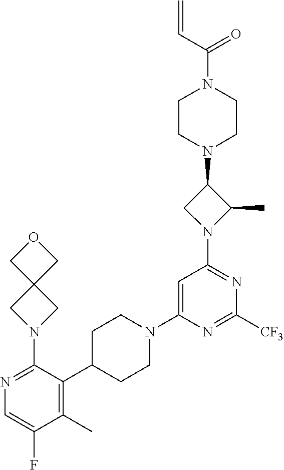 [SMILES: C=CC(=O)N1CCN([C@@H]2CN(c3cc(N4CCC(c5c(N6CC7(COC7)C6)ncc(F)c5C)CC4)nc(C(F)(F)F)n3)[C@@H]2C)CC1] | 1-(4-((2R,3R)-1-(6- (4-(5-fluoro-4- methyl-2-(2-oxa-6- azaspiro[3.3]beptan -6-yl)-3-pyridinyl)- 1-piperidinyl)-2- (trifluoromethyl)- 4-pyrimidinyl)-2- methyl-3- azetidinyl)-1- piperazinyl)-2- propen-1-one |
| | 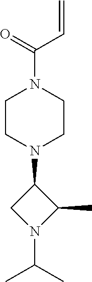 [SMILES: C=CC(=O)N1CCN([C@@H]2CN(C(C)C)[C@@H]2C)CC1] | | | | |
| 2-452 | 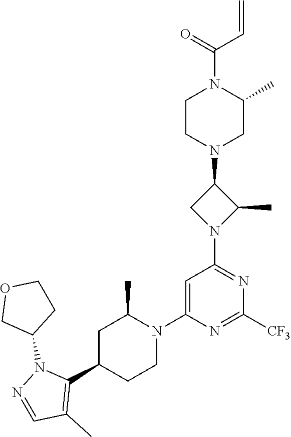 [SMILES: C=CC(=O)N1CCN([C@@H]2CN(c3cc(N4CC[C@@H](c5c(C)cnn5[C@H]5CCOC5)C[C@H]4C)nc(C(F)(F)F)n3)[C@@H]2C)C[C@H]1C] | 1-((2R)-2-methyl- 4-((2R,3R)-2- methyl-1-(6- ((2R,4R)-2-methyl- 4-(4-methyl-1- ((3S)-tetrahydro-3- furanyl)-1H- pyrazol-5-yl)-1- piperidiny])-2- (trifluoromethyl)- 4-pyrimidinyl)-3- azetidinyl)-1- piperazinyl)-2- propen-1-one | 2-456 | 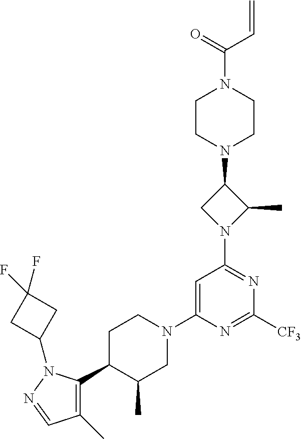 [SMILES: C=CC(=O)N1CCN([C@@H]2CN(c3cc(N4CC[C@H](c5c(C)cnn5C5CC(F)(F)C5)[C@H](C)C4)nc(C(F)(F)F)n3)[C@@H]2C)CC1] | 1-(4-((2R,3R)-1-(6- ((3S.4S)-4-(1-(3,3- difluorocyclo- butyl)-4-methyl- 1H-pyrazol-5-yl)- 3-methyl-1- piperidinyl)-2- (trifluoromethyl)- 4-pyrimidinyl)-2- methyl-3- azetidinyl)-1- piperazinyl)-2- propen-1-one |
| 2-453 | 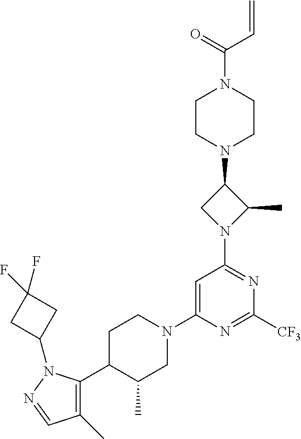 [SMILES: C=CC(=O)N1CCN([C@@H]2CN(c3cc(N4CCC(c5c(C)cnn5C5CC(F)(F)C5)[C@@H](C)C4)nc(C(F)(F)F)n3)[C@@H]2C)CC1] | 1-(4-((2R,3R)-1-(6- ((3R)-4-(1-(3,3- difluorocyclobutyl)- 4-methyl-1H- pyrazol-5-yl)-3- methylpiperidin-1- yl)-2- (trifluoromethyl) pyrimidin-4-yl)-2- methylazetidin-3- yl)piperazin-1- yl)prop-2-en-1-one | 2-457 | 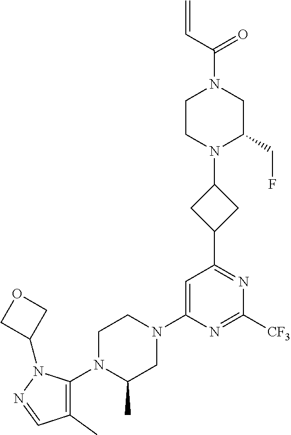 [SMILES: C=CC(=O)N1CCN(C2CC(c3cc(N4CCN(c5c(C)cnn5C5COC5)[C@H](C)C4)nc(C(F)(F)F)n3)C2)[C@@H](CF)C1] | 1-((R)-3- (fluoromethyl)-4- (3-(6-((R)-3- methyl-4-(4- methyl-1-(oxetan- 3-yl)-1H-pyrazol- 5-yl)piperazin-1- yl)-2- (trifluoromethyl) pyrimidin-4- yl)cyclobutyl) piperazin-1-yl) prop-2-en-1-one |
| | | | | mixture of trans isomers | |
| 2-454 | 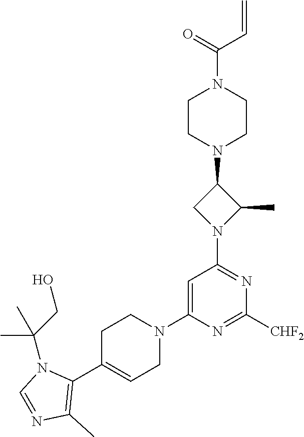 [SMILES: C=CC(=O)N1CCN([C@@H]2CN(c3cc(N4CC=C(c5c(C)ncn5C(C)(C)CO)CC4)nc(C(F)F)n3)[C@@H]2C)CC1] | 1-(4-((2R,3R)-1-(2- (difluoromethyl)-6- (4-(1-(1-hydroxy- 2-methyl-2- propanyl)-4- methyl-1H- pyrazol-5-yl)-3,6- dihydro-1(2H)- pyridinyl)-4- pyrimidinyl)-2- methyl-3- azetidinyl)-1- piperazinyl)-2- propen-1-one | 2-457-1 | 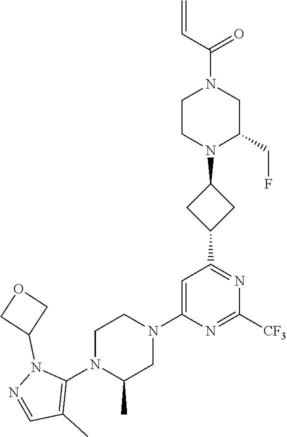 [SMILES: C=CC(=O)N1CCN([C@H]2C[C@H](c3cc(N4CCN(c5c(C)cnn5C5COC5)[C@H](C)C4)nc(C(F)(F)F)n3)C2)[C@@H](CF)C1] | 1-((3R)-3- (fluoromethyl)-4- (trans-3-(6-((3R)- 3-methyl-4-(4- methyl-1-(3- oxetanyl)-1H- pyrazol-5-yl)-1- piperazinyl)-2- (trifluoromethyl)- 4-pyrimidinyl) cyclobutyl)-1- piperazinyl)-2- propen-1-one |
| | | | | trans mixture | |
| 2-455 | 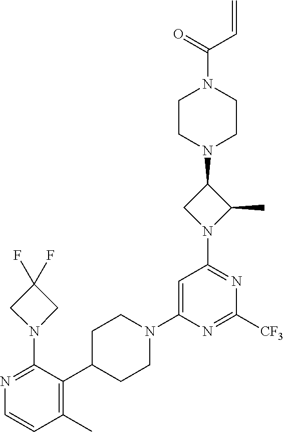 [SMILES: C=CC(=O)N1CCN([C@@H]2CN(c3cc(N4CCC(c5c(C)ccnc5N5CC(F)(F)C5)CC4)nc(C(F)(F)F)n3)[C@@H]2C)CC1] | 1-(4-((2R,3R)-1-(6- (4-(2-(3,3-difluoro- 1-azetidinyl)-4- methyl-3- pyridinyl)-1- piperidinyl)-2- (trifluoromethyl)- 4-pyrimidinyl)-2- methyl-3- azetidiny])-1- piperazinyl)-2- propen-1-one | 2-458 | 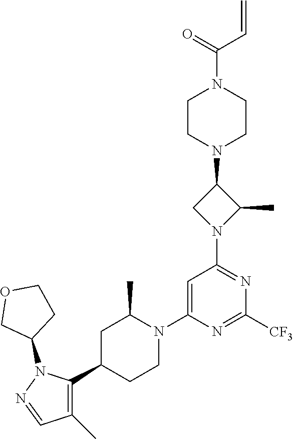 [SMILES: C=CC(=O)N1CCN([C@@H]2CN(c3cc(N4CC[C@@H](c5c(C)cnn5[C@@H]5CCOC5)C[C@H]4C)nc(C(F)(F)F)n3)[C@@H]2C)CC1] | 1-(4-((2R,3R)-2- methyl-1-(6- ((2R,4R)-2-methyl- 4-(4-methyl-1- ((3R)-tetrahydro-3- foranyl)-1H- pyrazol-5-yl)-1- piperidinyl)-2- (trifluoromethyl)- 4-pyrinuidinyl)-3- azetidinyl)-1- piperaziny])-2- propen-1-one |
| 2-459 | 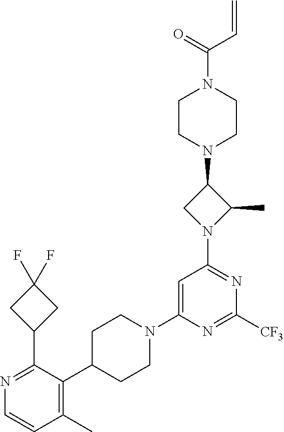 [SMILES: C=CC(=O)N1CCN([C@@H]2CN(c3cc(N4CCC(c5c(C)ccnc5C5CC(F)(F)C5)CC4)nc(C(F)(F)F)n3)[C@@H]2C)CC1] | 1-(4-((2R,3R)-1-(6- (4-(2-(3,3- difluorocyclobutyl)- 4-methyl-3- pyridinyl)-1- piperidinyl)-2- (trifluoromethyl)- 4-pyrimidinyl)-2- methyl-3- azetidinyl)-1- piperazinyl)-2- propen-1-one | 2-462-1 | 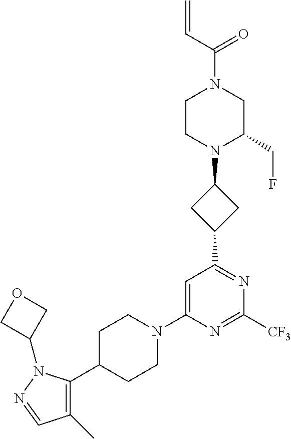 [SMILES: C=CC(=O)N1CCN([C@H]2C[C@H](c3cc(N4CCC(c5c(C)cnn5C5COC5)CC4)nc(C(F)(F)F)n3)C2)[C@@H](CF)C1] | 1-((3R)-3- (fluoromethyl)-4- (trans-3-(6-(4-(4- methyl-1-(3- oxetanyl)-1H- pyrazol-5-yl)-1- piperidinyl)-2- (trifluoromethyl)- 4-pyrimidinyl) cyclobutyl)-1- piperazinyl)-2- propen-1-one |
| | | | | trans mixture | |
| 2-460 | 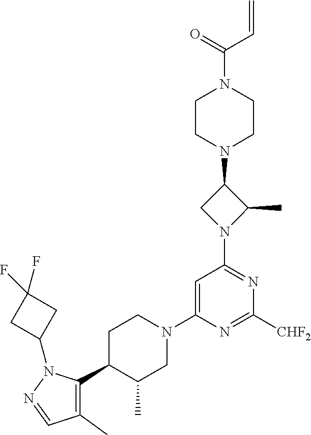 [SMILES: C=CC(=O)N1CCN([C@@H]2CN(c3cc(N4CC[C@H](c5c(C)cnn5C5CC(F)(F)C5)[C@@H](C)C4)nc(C(F)F)n3)[C@@H]2C)CC1] | 1-(4-((2R,3R)-1-(6- ((3R,4S)-4-(1-(3,3- difluorocyclobutyl)- 4-methyl-1H- pyrazol-5-yl)-3- methyl-1- piperidinyl)-2- (difluoromethyl)-4- pyrimidinyl)-2- methyl-3- azetidinyl)-1- piperazinyl)-2- propen-1-one | 2-463 | 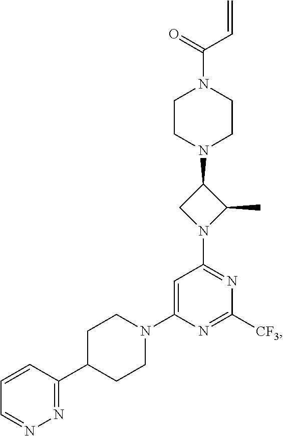 [SMILES: C=CC(=O)N1CCN([C@@H]2CN(c3cc(N4CCC(c5cccnn5)CC4)nc(C(F)(F)F)n3)[C@@H]2C)CC1] | 1-(4-((2R,3R)-2- methyl-1-(6-(4-(3- pyridazinyl)-1- piperidinyl)-2- (trifluoromethyl)- 4-pyrimidinyl)-3- azetidinyl)-1- piperazinyl)-2- propen-t-one |
| 2-461 | 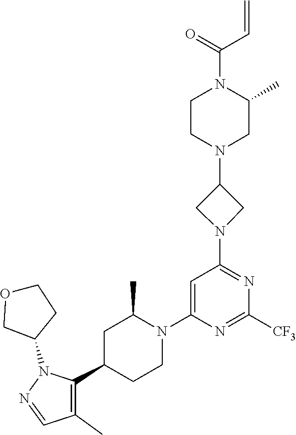 [SMILES: C=CC(=O)N1CCN(C2CN(c3cc(N4CC[C@@H](c5c(C)cnn5[C@H]5CCOC5)C[C@H]4C)nc(C(F)(F)F)n3)C2)C[C@H]1C] | 1-((2R)-2-methyl- 4-(1-(6-((2R,4R)-2- methyl-4-(4- methyl-1-((3S)- tetrahydro-3- furanyl)-1H- pyrazol-5-yl)-1- piperidinyl)-2- (trifluoromethyl)- 4-pyrimidinyl)-3- azetidinyl)-[- piperazinyl)-2- propen-1-one | 2-464 | 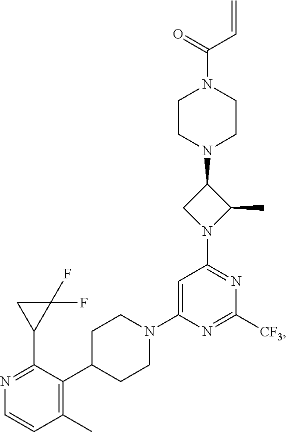 [SMILES: C=CC(=O)N1CCN([C@@H]2CN(c3cc(N4CCC(c5c(C)ccnc5C5CC5(F)F)CC4)nc(C(F)(F)F)n3)[C@@H]2C)CC1] | 1-(4-((2R,3R)-1-(6- (4-(2-(2,2- difluorocyclo- propyl)-4-methyl- 3-pyridinyl)-1- piperidinyl)-2- (trifluoromethyl)- 4-pyrimidinyl)-2- methyl-3- azetidinyl)-1- piperazinyl)-2- propen-1-one |
| 2-462 | 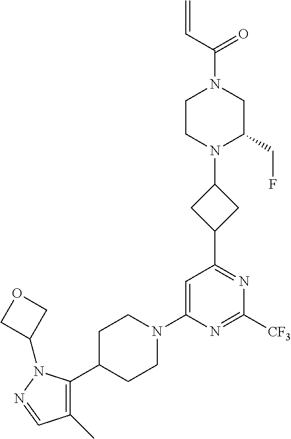 [SMILES: C=CC(=O)N1CCN(C2CC(c3cc(N4CCC(c5c(C)cnn5C5COC5)CC4)nc(C(F)(F)F)n3)C2)[C@@H](CF)C1] | (R)-1-(3- (fluoromethyl)-4- (3-(6-(4-(4-methyl- 1-(oxetan-3-yl)- 1H-pyrazol-5- yl)piperidin-1-yl)- 2-(trifluoromethyl) pyrimidin-4- yl)cyclobutyl)piper azin-1-yl)prop-2- en-1-one | 2-465 | 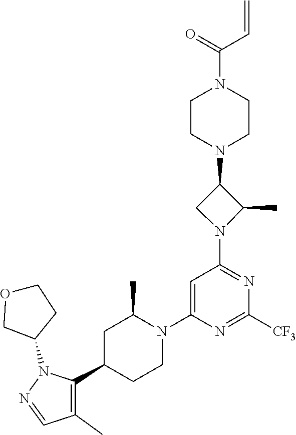 [SMILES: C=CC(=O)N1CCN([C@@H]2CN(c3cc(N4CC[C@@H](c5c(C)cnn5[C@H]5CCOC5)C[C@H]4C)nc(C(F)(F)F)n3)[C@@H]2C)CC1] | 1-(4-((2R,3R)-2- methyl-1-(6- ((2R,4R)-2-methyl- 4-(4-methyl-1- ((3S)-tetrahydro-3- foranyl)-1H- pyrazol-5-yl)-1- piperidinyl)-2- (trifluoromethyl)- 4-pyrimidinyl)-3- azetidinyl)-1- piperazinyl)-2- propen-1-one |
| | mixture of trans isomers | | | | |
| 2-466 | 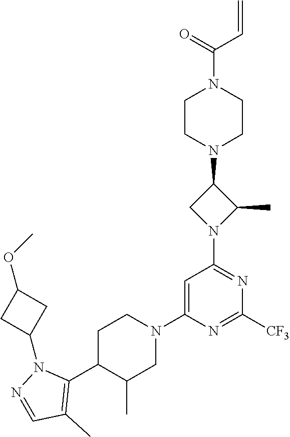 [SMILES: C=CC(=O)N1CCN([C@@H]2CN(c3cc(N4CCC(c5c(C)cnn5C5CC(OC)C5)C(C)C4)nc(C(F)(F)F)n3)[C@@H]2C)CC1] | 1-(4-((2R,3R)-1-(6- (4-(1-(3- methoxycyclo- butyl)-4-methyl-1H- pyrazol-5-yl)-3- methylpiperidin-1- yl)-2- (trifluoromethyl) pyrimidin-4-yl)-2- netbylazetidin-3- yl)prop-2-en-1-one | 2-468 | 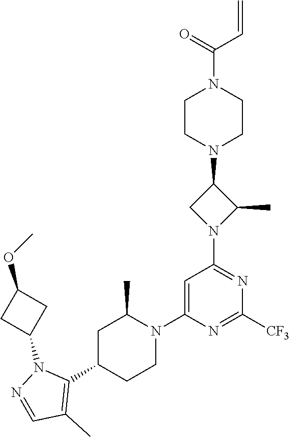 [SMILES: C=CC(=O)N1CCN([C@@H]2CN(c3cc(N4CC[C@H](c5c(C)cnn5[C@H]5C[C@H](OC)C5)C[C@H]4C)nc(C(F)(F)F)n3)[C@@H]2C)CC1] | 1-(4-((2R,3R)-1-(6- ((2R,4S)-4-(1- (trans-3- methoxycyclo- butyl)-4-methyl- 1H-pyrazol-5-yl)- 2-methyl-1- piperidinyl)-2- (trifluoromethyl)- 4-pyrimidinyl)-2- methyl-3- azetidinyl)-1- piperazinyl)-2- propen-1-one |
| 2-466-1 | 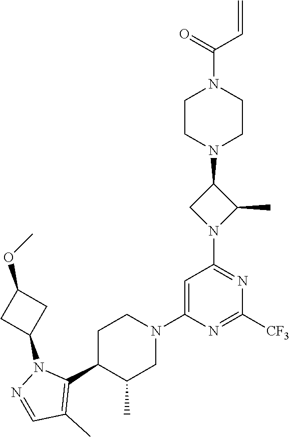 [SMILES: C=CC(=O)N1CCN([C@@H]2CN(c3cc(N4CC[C@H](c5c(C)cnn5[C@H]5C[C@@H](OC)C5)[C@@H](C)C4)nc(C(F)(F)F)n3)[C@@H]2C)CC1] | 1-(4-((2R.3R)-1-(6- ((3R,4S)-4-(1-(cis- 3-methoxycyclo butyl)-4-methyl-1H- pyrazol-5-yl)-3- methyl-1- piperidinyl)-2- (trifluoromethyl)- 4-pyrimidinyl)-2- methyl-3- azetidinyl)-1- piperazinyl)-2- propen-1-one | 2-469 | 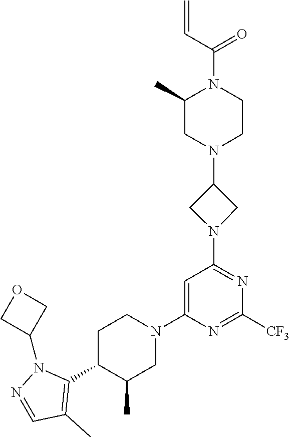 [SMILES: C=CC(=O)N1CCN(C2CN(c3cc(N4CC[C@@H](c5c(C)cnn5C5COC5)[C@H](C)C4)nc(C(F)(F)F)n3)C2)C[C@H]1C] | 1-((2R)-2-methyl- 4-(1-(6-((38,4R)- 3-methyl-4-(4- methyl-1-(3- oxetanyl)-1H- pyrazol-5-yl)-1- piperidinyl)-2- (trifluoromethyl)- 4-pyrimidinyl)-3- azetidinyl)-1- piperazinyl)-2- propen-1-one |
| | 1^{st}-eluting isomer | | | | |
| 2-466-2 | 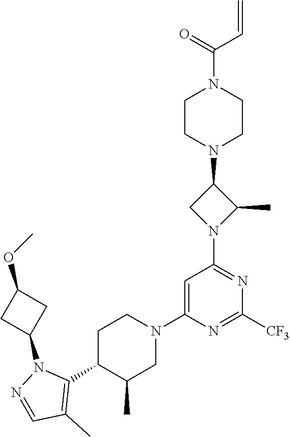 [SMILES: C=CC(=O)N1CCN([C@@H]2CN(c3cc(N4CC[C@@H](c5c(C)cnn5[C@H]5C[C@@H](OC)C5)[C@H](C)C4)nc(C(F)(F)F)n3)[C@@H]2C)CC1] | 1-(4-((2R,3R)-1-(6- ((3S,4R)-4-(1-(cis- 3-methoxycyclo- butyl)-4-methyl- 1H-pyrazol-5-yl)- 3-methyl-1- piperidinyl)-2- (trifluoromethyl)- 4-pyrimidinyl)-2- methyl-3- azetidinyl)-1- piperazinyl)-2- propen-1-one | 2-470 | 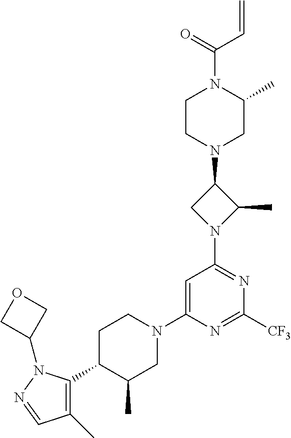 [SMILES: C=CC(=O)N1CCN([C@@H]2CN(c3cc(N4CC[C@@H](c5c(C)cnn5C5COC5)[C@H](C)C4)nc(C(F)(F)F)n3)[C@@H]2C)C[C@H]1C] | 1-((2R)-2-methyl- 4-((2R,3R)-2- methyl-1-(6- ((35,4R)-3-methyl- 4-(4-methyl-1-(3- oxetanyl)-1H- pyrazol-5-yl)-1- piperidinyl)-2- (trifluoromethyl)- 4-pyrimidinyl)-3- azetidinyl)-1- piperazinyl)-2- propen-1-one |
| | 2^{nd}-eluting isomer | | | | |
| 2-467 | 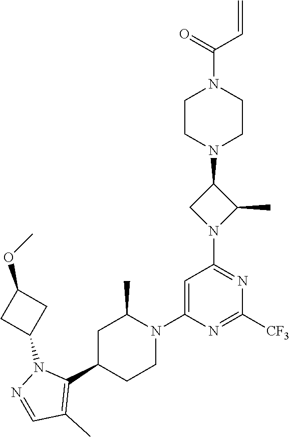 [SMILES: C=CC(=O)N1CCN([C@@H]2CN(c3cc(N4CC[C@@H](c5c(C)cnn5[C@H]5C[C@H](OC)C5)C[C@H]4C)nc(C(F)(F)F)n3)[C@@H]2C)CC1] | 1-(4-((2R,3R)-1-(6- ((2R,4R)-4-(1- (trans-3- methoxycyclo- butyl)-4-methyl- 1H-pyrazol-5-yl)- 2-methyl-1- piperidinyl)-2- (trifluoromethyl)- 4-pyrimidinyl)-2- methyl-3- azetidinyl)-1- piperazinyl)-2- propen-1-one | 2-471 | 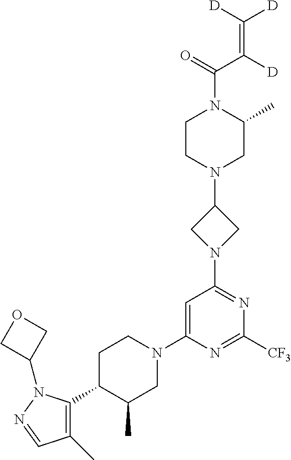 [SMILES: [2H]C([2H])=C([2H])C(=O)N1CCN(C2CN(c3cc(N4CC[C@@H](c5c(C)cnn5C5COC5)[C@H](C)C4)nc(C(F)(F)F)n3)C2)C[C@H]1C] | 1-((R)-2-methyl-4- (1-(6-((3S,4R)-3- methyl-4-(4- methyl-1-(oxetan- 3-yl)-1H-pyrazol- 5-yl)piperidin-1- yl)-2- (trifluoromethyl) pyrimidin-4- yl)azetidin-3- yl)piperazin-1- yl)prop-2-en-1- one-2,3,3-d; |
| 2-472 | 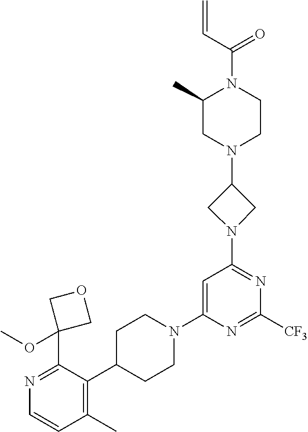 [SMILES: C=CC(=O)N1CCN(C2CN(c3cc(N4CCC(c5c(C)ccnc5C5(OC)COC5)CC4)nc(C(F)(F)F)n3)C2)C[C@H]1C] | 1-((2R)-4-(1-(6-(4- (2-(3-methoxy-3- oxetanyl)-4- methyl-3- pyridinyl)-1- piperidinyl)-2- (trifluoromethyl)- 4-pyrimidiny])-3- azetidinyl)-2- methyl-1- piperazinyl)-2- propen-1-one | 2-476 | 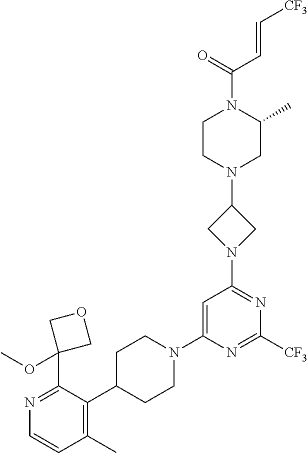 [SMILES: COC1(c2nccc(C)c2C2CCN(c3cc(N4CC(N5CCN(C(=O)/C=C/C(F)(F)F)[C@H](C)C5)C4)nc(C(F)(F)F)n3)CC2)COC1] | (2E)-4,4,4- trifluoro-1-((2R)-4- (1-(6-(4-(2-(3- methoxy-3- oxetanyl)-4- methyl-3- pyridinyl)-1- piperidinyl)-2- (trifluoromethyl)- 4-pyrimidinyl)-3- azetidinyl)-2- nethyl-1- piperazinyl)-2- buten-1-one |
| 2-473 | 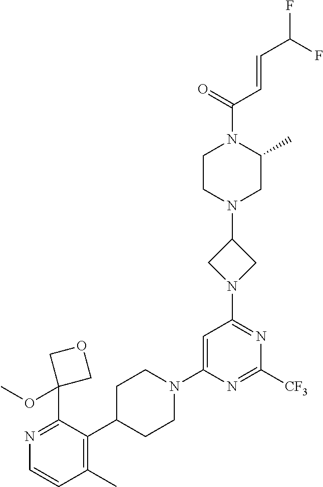 [SMILES: COC1(c2nccc(C)c2C2CCN(c3cc(N4CC(N5CCN(C(=O)/C=C/C(F)F)[C@H](C)C5)C4)nc(C(F)(F)F)n3)CC2)COC1] | (2E)-4,4-difluoro- 1-((2R)-4-(1-(6-(4- (2-(3-methoxy-3- oxetanyl)-4- methyl-3- pyridinyl)-1- piperidinyl)-2- (trifluoromethyl)- 4-pyrimidinyl)-3- azetidinyl)-2- methyl-1- piperazinyl)-2- buten-1-one | 2-477 | 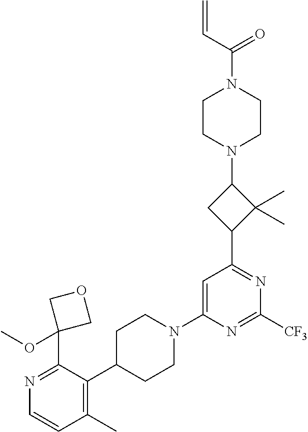 [SMILES: C=CC(=O)N1CCN(C2CC(c3cc(N4CCC(c5c(C)ccnc5C5(OC)COC5)CC4)nc(C(F)(F)F)n3)C2(C)C)CC1] | 1-(4-(-(3-(6-(4-(2- (3-methoxyoxetan- 3-yl)-4- methylpyridin-3- yl)piperidin-1-yl)- 2-(trifluoromethyl) pyrimidin-4-yl)-2,2- dimethylcyclo- butyl)piperazin-1- yl)prop-2-en-1-one |
| | | | | mixture of cis Isomers | |
| 2-474 | 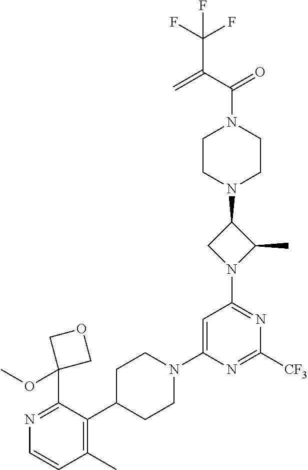 [SMILES: C=C(C(=O)N1CCN([C@@H]2CN(c3cc(N4CCC(c5c(C)ccnc5C5(OC)COC5)CC4)nc(C(F)(F)F)n3)[C@@H]2C)CC1)C(F)(F)F] | 1-(4-((2R,3R)-1-(6- (4-(2-(3-methoxy- 3-oxetanyl)-4- methyl-3- pyridinyl)-1- piperidinyl)-2- (trifluoromethy])- 4-pyrimidinyl)-2- methyl-3- azetidinyl)-1- piperazinyl)-2- (trifluoromethyl)- 2-propen-1-one | 2-477-1 | 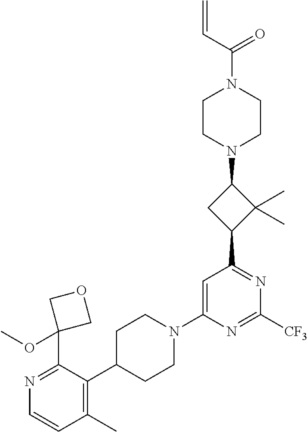 [SMILES: C=CC(=O)N1CCN([C@@H]2C[C@H](c3cc(N4CCC(c5c(C)ccnc5C5(OC)COC5)CC4)nc(C(F)(F)F)n3)C2(C)C)CC1] | 1-(4-((1R,3S)-3-(6- (4-(2-(3-methoxy- 3-oxetanyl)-4- methyl-3- pyridinyl)-1- piperidiny])-2- (trifluoromethyl)- 4-pyrimidinyl)-2,2- dimethylcyclo- butyl)-1- piperazinyl)-2- propen-1-one |
| | | | | 2^{nd}-eluting isomer | |
| 2-475 | 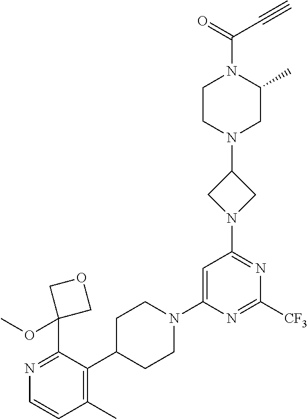 [SMILES: C#CC(=O)N1CCN(C2CN(c3cc(N4CCC(c5c(C)ccnc5C5(OC)COC5)CC4)nc(C(F)(F)F)n3)C2)C[C@H]1C] | 1-((2R)-4-(1-(6-(4- (2-(3-methoxy-3- oxetanyI)-4- methyl-3- pyridinyl)-1- piperidinyl)-2- (trifluoromethyl)- 4-pyrimidinyl)-3- azetidinyl)-2- methyl-1- piperazinyl)-2- propyn-1-one | 2-478 | 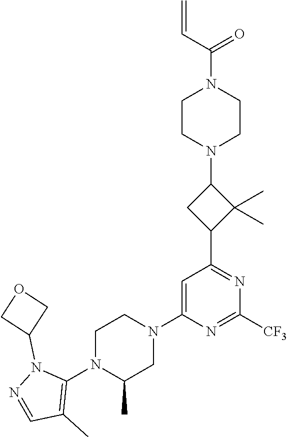 [SMILES: C=CC(=O)N1CCN(C2CC(c3cc(N4CCN(c5c(C)cnn5C5COC5)[C@H](C)C4)nc(C(F)(F)F)n3)C2(C)C)CC1] | 1-(4-(2,2-dimethyl- 3-(6-((R)-3- methyl-4-(4- methyl-1-(oxetan- 3-yl)-1H-pyrazol- 5-yl)piperazin-1- yl)-2- (trifluoromethyl) pyrimidin-4- yl)cyclobutyl) piperazin-1- yl)prop-2- en-1-one |
| | | | | mixture of cis isomers | |
| 2-478-1 | 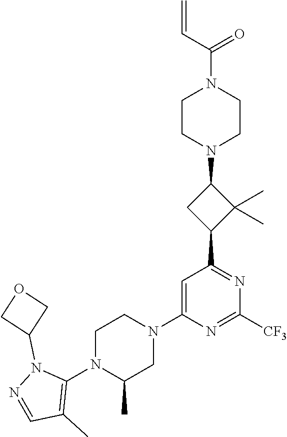 [SMILES: C=CC(=O)N1CCN([C@@H]2C[C@H](c3cc(N4CCN(c5c(C)cnn5C5COC5)[C@H](C)C4)nc(C(F)(F)F)n3)C2(C)C)CC1] | 1-(4-((IR,3S)-2,2- dimethyl-3-(6- ((3R)-3-methyl-4- (4-methyl-1-(3- oxetanyl)-1H- pyrazol-5-yl)-1- piperazinyl)-2- (trifluoromcthyl)- pyrimidinyl) cyclobutyl)-1- piperazinyl)-2- propen-1-one | 2-481 | 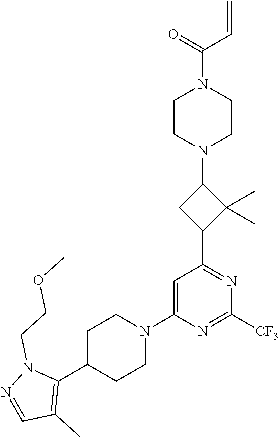 [SMILES: C=CC(=O)N1CCN(C2CC(c3cc(N4CCC(c5c(C)cnn5CCOC)CC4)nc(C(F)(F)F)n3)C2(C)C)CC1] | 1-(4-(3-(6-(4-(1-(2- methoxyethyl)-4- methyl-1H- pyrazol-5-yl)-1- piperidinyl)-2- (trifluoromethyl)- 4-pyrimidiny])-2,2- dimethylcyclo- butyl)-1- piperazinyl)-2- propen-1-one |
| | 2^{nd}-eluting isomer | | | mixture of cis isomers | |
| 2-479 | 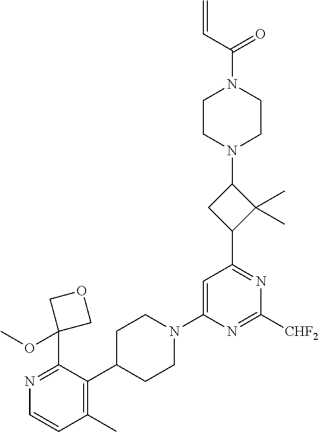 [SMILES: C=CC(=O)N1CCN(C2CC(c3cc(N4CCC(c5c(C)ccnc5C5(OC)COC5)CC4)nc(C(F)F)n3)C2(C)C)CC1] | 1-(4-(3-(2- (difluoromethyl)-6- (4-(2-(3- methoxyoxetan-3- yl)-4- methylpyridin-3- yl)piperidin-1- yl)pyrimidin-4-yl)- 2,2- dimethylcyclo- butyl)piperazin-1- yl)prop-2-en-1-one | 2-482 | 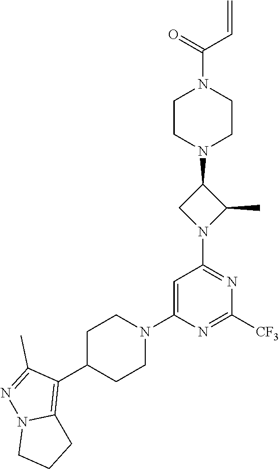 [SMILES: C=CC(=O)N1CCN([C@@H]2CN(c3cc(N4CCC(c5c(C)nn6c5CCC6)CC4)nc(C(F)(F)F)n3)[C@@H]2C)CC1] | 1-(4-((2R,3R)-2- methyl-1-(6-(4-(2- methyl-5,6- dihydro-4H- pyrrolo[1,2- bjpyrazol-3-yl)-1- piperidinyl)-2- (trifluoromethyl)- 4-pyrimidinyl)-3- azetidinyl)-1- piperazinyl)-2- propen-1-one |
| | mixture of cis isomers | | | | |
| 2-479-1 | 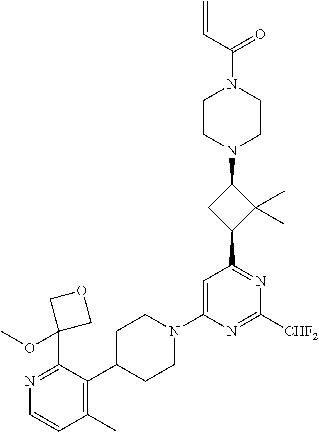 [SMILES: C=CC(=O)N1CCN([C@@H]2C[C@H](c3cc(N4CCC(c5c(C)ccnc5C5(OC)COC5)CC4)nc(C(F)F)n3)C2(C)C)CC1] | 1-(4-((1R,3S)-3-(2- (difluoromethyl)-6- (4-(2-(3-methoxy- 3-oxetanyl)-4- methyl-3- pyridinyl)-1- piperidinyl)-4- pyrimidinyl)-2,2- dimethylcyclo- butyl)-1- piperazinyl)-2- propen-1-one | 2-483 | 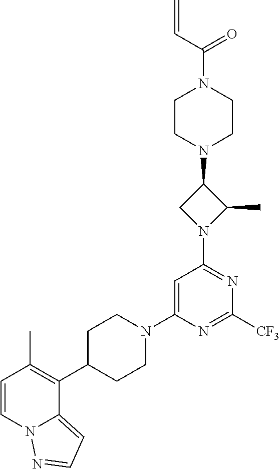 [SMILES: C=CC(=O)N1CCN([C@@H]2CN(c3cc(N4CCC(c5c(C)ccn6nccc56)CC4)nc(C(F)(F)F)n3)[C@@H]2C)CC1] | 1-(4-((2R,3R)-2- methyl-1-(6-(4-(5- methylpyrazolo [1,5-a]pyridin-4- yl)-1- piperidinyl)-2- (trifluoromethyl)- 4-pyrimidinyl)-3- azetidinyl)-1- piperazinyl)-2- propon-t-one |
| | 1^{st}-eluting isomer | | | | |
| 2-480 | 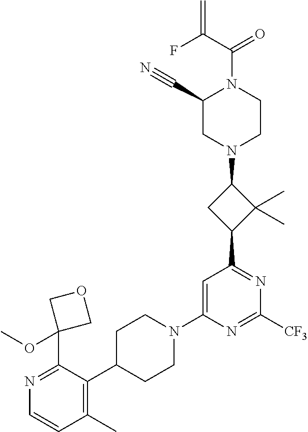 [SMILES: C=C(F)C(=O)N1CCN([C@@H]2C[C@H](c3cc(N4CCC(c5c(C)ccnc5C5(OC)COC5)CC4)nc(C(F)(F)F)n3)C2(C)C)C[C@H]1C#N] | (2S)-1-(2-fluoro-2- propenoyl)-4- ((1R,3S)-3-(6-(4- (2-(3-methoxy-3- oxetanyl)-4- methyl-3- pyridinyl)-1- piperidiny])-2- (trifluoromethyl)- 4-pyrimidinyl)-2,2- dimethylcyclo- butyl)-2- piperazine- carbonitrile | 2-484 | 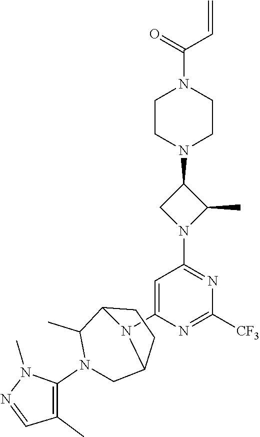 [SMILES: C=CC(=O)N1CCN([C@@H]2CN(c3cc(N4C5CCC4C(C)N(c4c(C)cnn4C)C5)nc(C(F)(F)F)n3)[C@@H]2C)CC1] | 1-(4-((2R,3R)-1-(6- (3-(1,4-dimethyl- 1H-pyrazol-5-yl)- 2-methyl-3,8- diazabicyclo[3.2.1] octan-8-y])-2- (trifluoromethyl)- 4-pyrimidinyl)-2- methyl-3- azetidinyl)-1- piperazinyl)-2- propen-1-one |
| 2-485 | 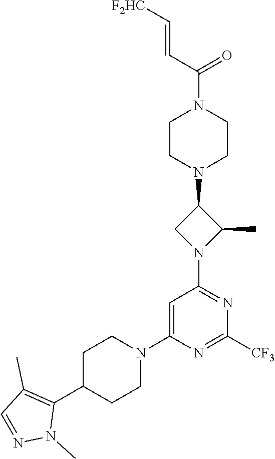 [SMILES: Cc1cnn(C)c1C1CCN(c2cc(N3C[C@@H](N4CCN(C(=O)/C=C/C(F)F)CC4)[C@H]3C)nc(C(F)(F)F)n2)CC1] | (2E)-1-(4- ((2R,3R)-1-(6-(4- (1,4-dimethyl-1H- pyrazol-5-yl)-1- piperidinyl)-2- (trifluoromethyl)- 4-pynimidinyl)-2- methyl-3- azetidinyl)-1- piperazinyl)-4,4- difluoro-2-buten-1- one | 2-489 | 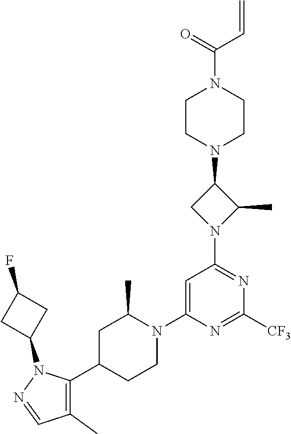 [SMILES: C=CC(=O)N1CCN([C@@H]2CN(c3cc(N4CCC(c5c(C)cnn5[C@H]5C[C@@H](F)C5)C[C@H]4C)nc(C(F)(F)F)n3)[C@@H]2C)CC1] | 1-(4-((2R,3R)-1-(6- ((2R)-4-(1- ((1s,3S)-3- fluorocyclobutyl)- 4-methyl-1H- pyrazol-5-yl)-2- methylpiperidin-1- yl)-2- (trifluoromethyl) pyrimidin-4-yl)-2- methylazetidin-3- ylpiperazin-1- yl)prop-2-en-1-one |
| | | | | mixture of isomers | |
| 2-486 | 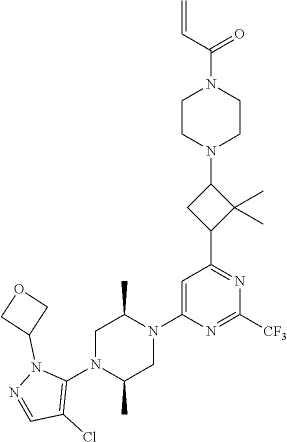 [SMILES: C=CC(=O)N1CCN(C2CC(c3cc(N4C[C@@H](C)N(c5c(Cl)cnn5C5COC5)C[C@H]4C)nc(C(F)(F)F)n3)C2(C)C)CC1] | 1-(4-(3-(6- ((2R,SR)-4-(4- chloro-1-(3- oxetanyl)-1H- pyrazol-S-yl)-2,5- dimethyl-1- piperazinyl)-2- (trifluoromethyl)- 4-pyrimidinyl)-2,2- dimethylcyclo- butyl)-1- piperazinyl)-2- propen-1-one | 2-489-1 | 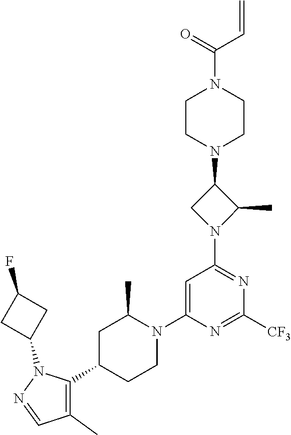 [SMILES: C=CC(=O)N1CCN([C@@H]2CN(c3cc(N4CC[C@H](c5c(C)cnn5[C@H]5C[C@H](F)C5)C[C@H]4C)nc(C(F)(F)F)n3)[C@@H]2C)CC1] | 1-(4-((2R,3R)-1-(6- ((2R,4S)-4-(1- (trans-3- fluorocyclobutyl)- 4-methyl-1H- pyrazol-5-yl)-2- methyl-1- piperidinyl)-2- (trifluoromethyl)- 4-pyrimidinyl)-2- methyl-3- azetidinyl)-1- piperazinyl)-2- propen-1-one |
| | mixture of cis isomers | | | 1^{st}-eluting isomer | |
| 2-487 | 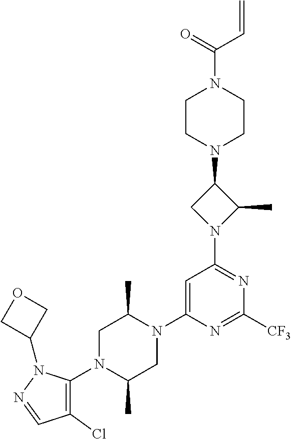 [SMILES: C=CC(=O)N1CCN([C@@H]2CN(c3cc(N4C[C@@H](C)N(c5c(Cl)cnn5C5COC5)C[C@H]4C)nc(C(F)(F)F)n3)[C@@H]2C)CC1] | 1-(4-((2R,3R)-1-(6- ((2R,5R)-4-(4- chloro-1-(3- oxetanyl)-1H- pyrazol-5-yl)-2,5- dimethyl-1- piperazinyl)-2- (trifluoromethyl)- 4-pyrimidinyl)-2- methyl-3- azetidinyl)-1- piperazinyl)-2- propen-1-one | 2-490 | 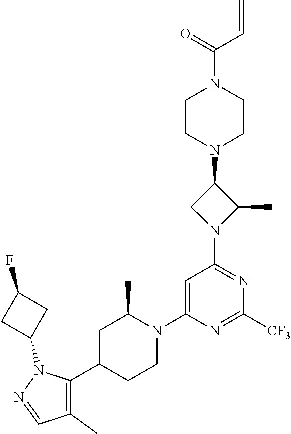 [SMILES: C=CC(=O)N1CCN([C@@H]2CN(c3cc(N4CCC(c5c(C)cnn5[C@H]5C[C@H](F)C5)C[C@H]4C)nc(C(F)(F)F)n3)[C@@H]2C)CC1] | 1-(4-((2R,3R)-1-(6- ((2R)-4-(1- ((Ir,3R)-3- fluorocyclobutyl)- 4-methyl-1H- pyrazol-5-yl)-2- methylpiperidin-1- yl)-2- (trifluoromethyl) pyrimidin-4-yl)-2- methylazetidin-3- yl)piperazin-1- yl)prop-2-en-1-one |
| | | | | mixture of isomers | |
| 2-488 | 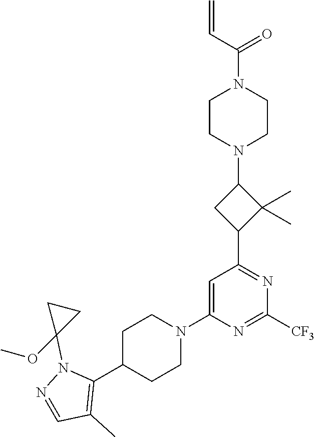 [SMILES: C=CC(=O)N1CCN(C2CC(c3cc(N4CCC(c5c(C)cnn5C5(OC)CC5)CC4)nc(C(F)(F)F)n3)C2(C)C)CC1] | 1-(4-(3-(6-(4-(1-(1- methoxycyclo- propyl)-4-methyl- 1H-pyrazol-5-yl)- 1-piperidinyl)-2- (trifluoromethyl)- 4-pyrimidinyl)-2,2- dimethylcyclo- butyl)-1- piperaziny])-2- propen-1-one | 2-490-1 | 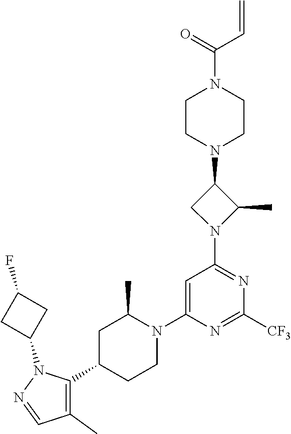 [SMILES: C=CC(=O)N1CCN([C@@H]2CN(c3cc(N4CC[C@H](c5c(C)cnn5[C@H]5C[C@@H](F)C5)C[C@H]4C)nc(C(F)(F)F)n3)[C@@H]2C)CC1] | 1-(4-((2R,3R)-1-(6- ((2R,4S)-4-(1- (cis-3- fluorocyclobntyl)- 4-methyl-1H- pyrazol-5-yl)-2- methyl-1- piperidinyl)-2- (trifluoromethyl)- 4-pyrimidinyl)-2- methyl-3- azetidinyl)-1- piperazinyl)-2- propen-1-one |
| | racemic mixture | | | 1^{st}-eluting isomer | |
| 2-491 | 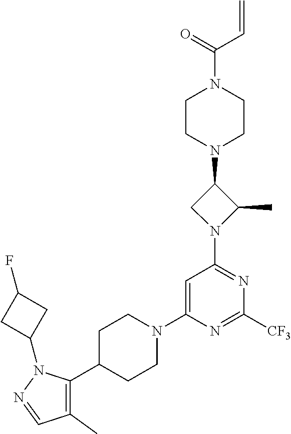 [SMILES: C=CC(=O)N1CCN([C@@H]2CN(c3cc(N4CCC(c5c(C)cnn5C5CC(F)C5)CC4)nc(C(F)(F)F)n3)[C@@H]2C)CC1] | 1-(4-((2R,3R)-1-(6- (4-(1-3- fluorocyclobutyl)- 4-methyl-1H- pyrazol-5-yl)-1- piperidinyl)-2- (trifluoromethyl)- 4-pyrimidiny])-2- methyl-3- azetidinyl)-1- piperazinyl)-2- propen-1-one | 2-492-1 | 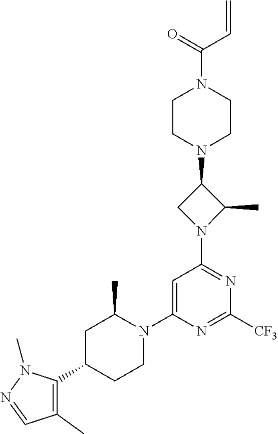 [SMILES: C=CC(=O)N1CCN([C@@H]2CN(c3cc(N4CC[C@H](c5c(C)cnn5C)C[C@H]4C)nc(C(F)(F)F)n3)[C@@H]2C)CC1] | 1-(4-((2R,3R)-1-(6- ((2R.4S)-4-(1,4- dimethyl-1H- pyrazol-5-yl)-2- methyl-1- piperidinyl)-2- (trifluoromethyl)- 4-pyrimidinyl)-2- methyl-3- azetidinyl)-1- piperazinyl)-2- propen-1-one |
| | | | | 1^{st}-eluting isomer | |
| 2-491-1 | 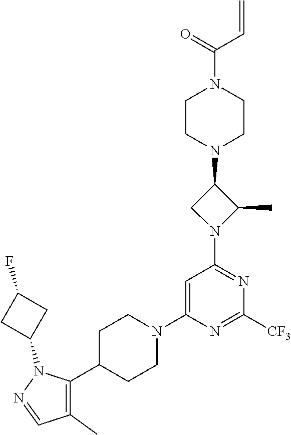 [SMILES: C=CC(=O)N1CCN([C@@H]2CN(c3cc(N4CCC(c5c(C)cnn5[C@H]5C[C@@H](F)C5)CC4)nc(C(F)(F)F)n3)[C@@H]2C)CC1] | 1-(4-((2R,3R)-1-(6- (4-(1-(cis-3- fluorocyclobutyl)- 4-methyl-1H- pyrazol-5-yl)-1- piperidiny])-2- (trifluoromethyl)- 4-pyrimidinyl)-2- methyl-3- azetidinyl)-1- piperazinyl)-2- propen-1-one | 2-493 | 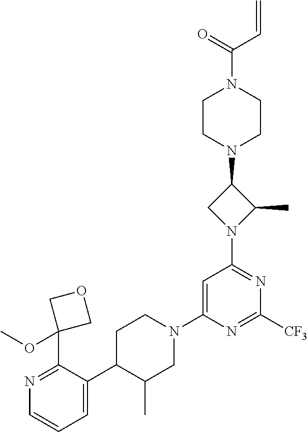 [SMILES: C=CC(=O)N1CCN([C@@H]2CN(c3cc(N4CCC(c5cccnc5C5(OC)COC5)C(C)C4)nc(C(F)(F)F)n3)[C@@H]2C)CC1] | 1-(4-(2R,3R)-1-(6- (4-(2-(3-methoxy- 3-oxetanyl)-3- pyridinyl)-3- methyl-1- piperidiny])-2- (trifluoromethyl)- 4-pyrimidinyl)-2- methyl-3- azetidinyl)-1- piperazinyl)-2- propen-1-one |
| | 1^{st}-eluting isomer | | | | |
| 2-491-2 | 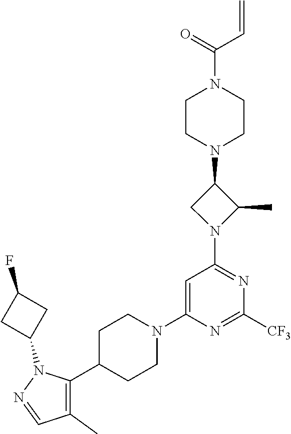 [SMILES: C=CC(=O)N1CCN([C@@H]2CN(c3cc(N4CCC(c5c(C)cnn5[C@H]5C[C@H](F)C5)CC4)nc(C(F)(F)F)n3)[C@@H]2C)CC1] | 1-(4-((2R,3R)-1-(6- (4-(1-(trans-3- fluorocyclobutyl)- 4-methyl-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-493-1 | 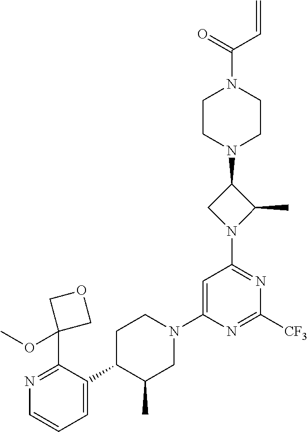 [SMILES: C=CC(=O)N1CCN([C@@H]2CN(c3cc(N4CC[C@@H](c5cccnc5C5(OC)COC5)[C@H](C)C4)nc(C(F)(F)F)n3)[C@@H]2C)CC1] | 1-(4-((2R,3R)-1-(6- ((3S,4R)-4-(2-(3- methoxy-3- oxetanyl)-3- pyridinyl)-3- methyl-1- piperidinyl)-2- (trifluoromethyl)- 4-pyrimidinyl)-2- methyl-3- azetidinyl)-1- piperazinyl)-2- propen-1-one |
| | 2^{nd}-eluting isomer | | | 1^{st}-eluting isomer | |
| 2-492 | 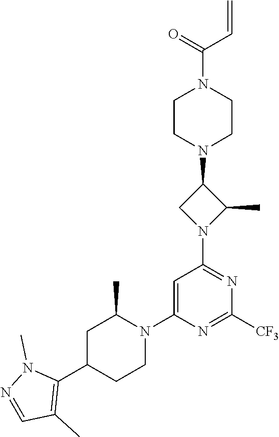 [SMILES: C=CC(=O)N1CCN([C@@H]2CN(c3cc(N4CCC(c5c(C)cnn5C)C[C@H]4C)nc(C(F)(F)F)n3)[C@@H]2C)CC1] | 1-(4-((2R,3R)-1-(6- ((2R)-4-(1,4- dimethyl-1H- pyrazol-5-yl)-2- yl)-2- (trifluoromethyl) pyrimidin-4-yl)-2- methylazetidin-3- yl)piperazin-1- yl)prop-2-en-1-one | 2-493-2 | 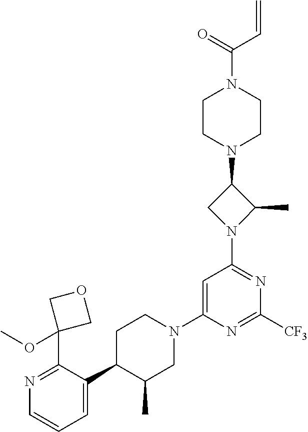 [SMILES: C=CC(=O)N1CCN([C@@H]2CN(c3cc(N4CC[C@H](c5cccnc5C5(OC)COC5)[C@H](C)C4)nc(C(F)(F)F)n3)[C@@H]2C)CC1] | 1-(4-((2R,3R)-1-(6- ((3S,4S)-4-(2-(3- methoxy-3- oxetanyl)-3- pyridinyl)-3- methyl-1- piperidinyl)-2- (trifluoromethyl)- 4-pyrimidinyl)-2- methyl-3- azetidinyl)-1- piperaziny])-2- propen-1-one |
| | | | | 3rd-eluting isomer | |
| 2-493-3 | 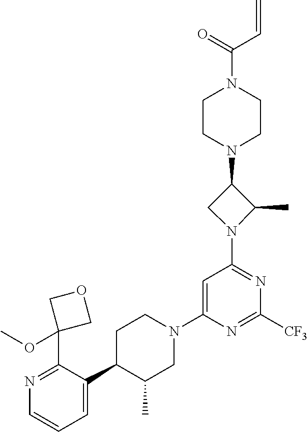 [SMILES: C=CC(=O)N1CCN([C@@H]2CN(c3cc(N4CC[C@H](c5cccnc5C5(OC)COC5)[C@@H](C)C4)nc(C(F)(F)F)n3)[C@@H]2C)CC1] | 1-(4-((2R,3R)-1-(6- ((3R,4S)-4-(2-(3- methoxy-3- oxetanyl)-3- pyridinyl)-3- methyl-1- piperidinyl)-2- (trifluoromethyl)- 4-pyrimidinyl)-2- methyl-3- azetidinyl)-1- piperazinyl)-2- propen-1-one | 2-495-1 | 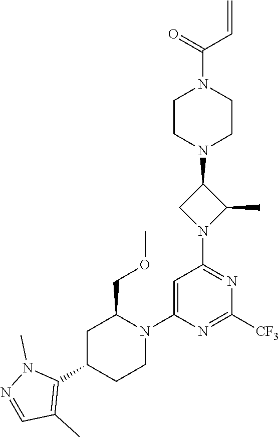 [SMILES: C=CC(=O)N1CCN([C@@H]2CN(c3cc(N4CC[C@H](c5c(C)cnn5C)C[C@H]4COC)nc(C(F)(F)F)n3)[C@@H]2C)CC1] | 1-(4-((2R,3R)-1-(6- ((2S,4S)-4-(1,4- dimethyl-1H- pyrazol-5-yl)-2- (methoxymethyl)- 1-piperidinyl)-2- (trifluoromethyl)- 4-pyrimidinyl)-2- methyl-3- azetidinyl)-1- piperazinyl)-2- propen-1-one |
| | 4^{th}-eluting isomer | | | 2^{nd}-eluting isomer | |
| 2-494 | 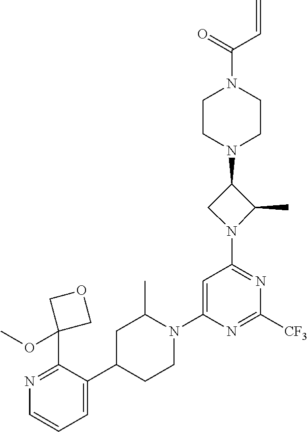 [SMILES: C=CC(=O)N1CCN([C@@H]2CN(c3cc(N4CCC(c5cccnc5C5(OC)COC5)CC4C)nc(C(F)(F)F)n3)[C@@H]2C)CC1] | 1-(4-((2R,3R)-1-(6- (4-(2-(3-methoxy- 3-oxetanyl)-3- pyridinyl)-2- methyl-1- piperidinyl)-2- (trifluoromethyl)- 4-pyrimidinyl)-2- methyl-3- azetidinyl)-1- piperazinyl)-2- propen-1-one | 2-496 | 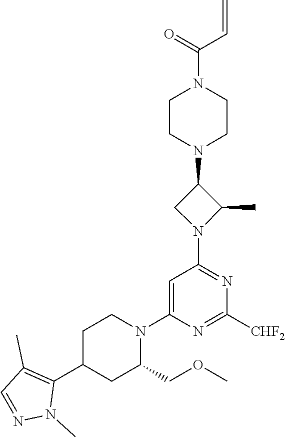 [SMILES: C=CC(=O)N1CCN([C@@H]2CN(c3cc(N4CCC(c5c(C)cnn5C)C[C@H]4COC)nc(C(F)F)n3)[C@@H]2C)CC1] | 1-(4-((2R,3R)-1-(2- (difluoromethyl)-6- ((2S)-4-(1,4- dimethyl-1H- pyrazol-5-yl)-2- (methoxymethyl) piperidin-1- yl)pyrimidin-4-yl)- 2-methylazetidin- 3-yl)piperazin-1- yl)prop-2-en-1-one |
| 2-494-1 | 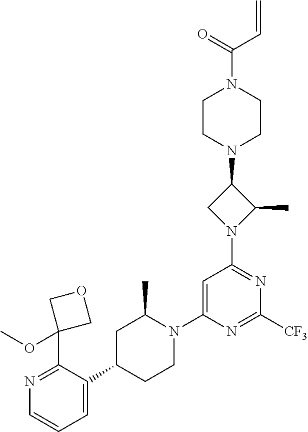 [SMILES: C=CC(=O)N1CCN([C@@H]2CN(c3cc(N4CC[C@H](c5cccnc5C5(OC)COC5)C[C@H]4C)nc(C(F)(F)F)n3)[C@@H]2C)CC1] | 1-(4-((2R,3R)-1-(6- ((2R,45)-4-(2-(3- methoxy-3- oxetanyl)-3- pyridinyl)-2- methyl-1- piperidiny])-2- (trifluoromethyl)- 4-pyrimidinyl)-2- methyl-3- azetidinyl)-1- piperazinyl)-2- propen-1-one | 2-496-1 | 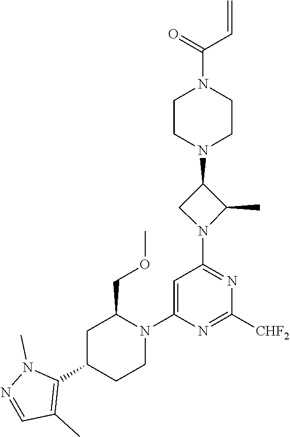 [SMILES: C=CC(=O)N1CCN([C@@H]2CN(c3cc(N4CC[C@H](c5c(C)cnn5C)C[C@H]4COC)nc(C(F)F)n3)[C@@H]2C)CC1] | 1-(4-((2R,3R)-1-(2- (difluorometliy])-6- ((2S,4S)-4-(1,4- dimethyl-1H- pyrazol-5-yl)-2- (methoxymethyl)- I-piperidinyl)-4- pyrimidinyl)-2- methyl-3- azetidinyl)-1- piperazinyl)-2- propen-1-one |
| | 1^{st}-eluting isomer | | | 3^{rd}-eluting isomer | |
| 2-495 | 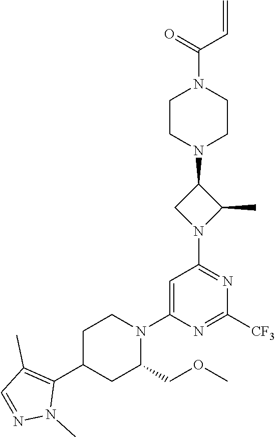 [SMILES: C=CC(=O)N1CCN([C@@H]2CN(c3cc(N4CCC(c5c(C)cnn5C)C[C@H]4COC)nc(C(F)(F)F)n3)[C@@H]2C)CC1] | 1-(4-((2R,3R)-1-(6- ((2S)-4-(1,4- dimethyl-1H- pyrazol-5-yl)-2- (methoxymethyl) piperidin-1-yl)-2- (trifluoromethyl) pyrimidin-4-yl)-2- methylazetidin-3- yl)piperazin-1- yl)prop-2-en-1-one | 2-497 | 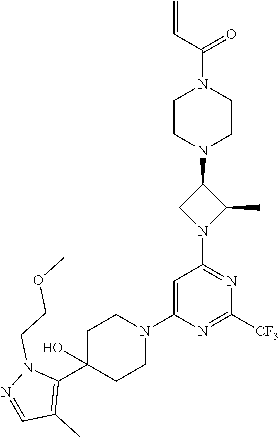 [SMILES: C=CC(=O)N1CCN([C@@H]2CN(c3cc(N4CCC(O)(c5c(C)cnn5CCOC)CC4)nc(C(F)(F)F)n3)[C@@H]2C)CC1] | 1-(4-((2R,3R)-1-(6- (4-hydroxy-4-(1- (2-methoxyethyl)- 4-methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-498 | 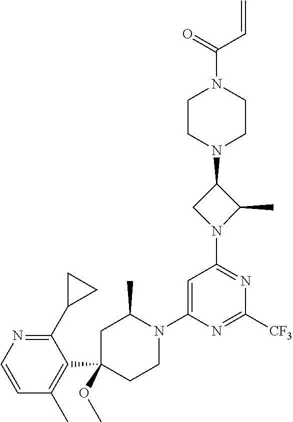 [SMILES: C=CC(=O)N1CCN([C@@H]2CN(c3cc(N4CC[C@](OC)(c5c(C)ccnc5C5CC5)C[C@H]4C)nc(C(F)(F)F)n3)[C@@H]2C)CC1] | 1-(4-((2R,3R)-1-(6- ((2R.4R)-4-(2- cyclopropyl-4- methyl-3- pyridinyl)-4- methoxy-2-methyl- 1-piperidinyl)-2- (trifluoromethyl)- 4-pyrimidinyl)-2- methyl-3- azetidinyl)-11 piperazinyl)-2- propen-1-one | 2-502 | 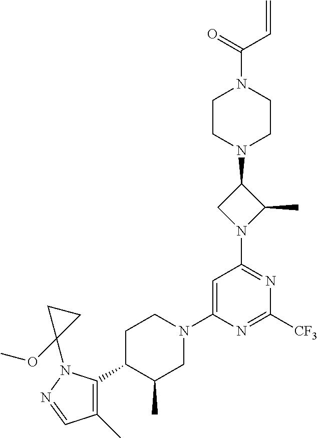 [SMILES: C=CC(=O)N1CCN([C@@H]2CN(c3cc(N4CC[C@@H](c5c(C)cnn5C5(OC)CC5)[C@H](C)C4)nc(C(F)(F)F)n3)[C@@H]2C)CC1] | 1-(4-((2R,3R)-1-(6- ((3S,4R)-4-(1-(1- methoxycyclo- propyl)-4-methyl- 1H-pyrazol-5-yl)- 3-methyl-1- piperidinyl)-2- (trifluoromethyl)- 4-pyrimidinyl)-2- methyl-3- azetidinyl)-1- piperazinyl)-2- propen-1-one |
| 2-499 | 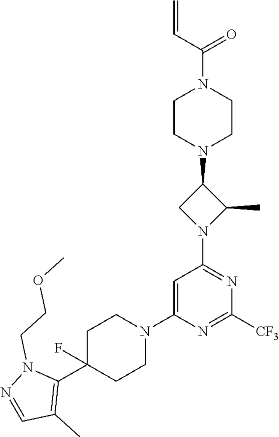 [SMILES: C=CC(=O)N1CCN([C@@H]2CN(c3cc(N4CCC(F)(c5c(C)cnn5CCOC)CC4)nc(C(F)(F)F)n3)[C@@H]2C)CC1] | 1-(4-((2R,3R)-1-(6- (4-fluoro-4-(1-(2- methoxyethyl)-4- methyl-1H- pyrazol-5-yl)-1- piperidiny])-2- (trifluoromethyl)- 4-pyrimidinyl)-2- methyl-3- azetidinyl)-1- piperazinyl)-2- propen-1-one | 2-503 | 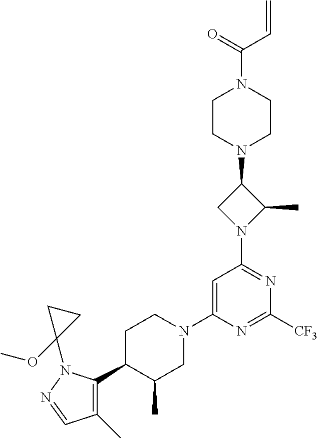 [SMILES: C=CC(=O)N1CCN([C@@H]2CN(c3cc(N4CC[C@H](c5c(C)cnn5C5(OC)CC5)[C@H](C)C4)nc(C(F)(F)F)n3)[C@@H]2C)CC1] | 1-(4-((2R,3R)-1-(6- ((3S.45)-4-(1-(1- methoxycyclo- propyl)-4- methyl-1H- pyrazol-5-yl)-3- methyl-1- piperidinyl)-2- (trifluoromethyl)- 4-pyrimidinyl)-2- methyl-3- azetidinyl)-]- piperazinyl)-2- propen-1-one |
| 2-500 | 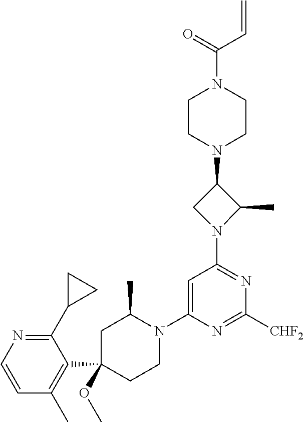 [SMILES: C=CC(=O)N1CCN([C@@H]2CN(c3cc(N4CC[C@](OC)(c5c(C)ccnc5C5CC5)C[C@H]4C)nc(C(F)F)n3)[C@@H]2C)CC1] | 1-(4-((2R,3R)-1-(6- ((2R,4R)-4-(2- cyclopropyl-4- methyl-3- pyridinyl)-4- methoxy-2-methyl- 1-piperidinyl)-2- (difluoromethyl)-4- pyrimidinyl)-2- methyl-3- azetidinyl)-1- piperazinyl)-2- propen-1-one | 2-504 | 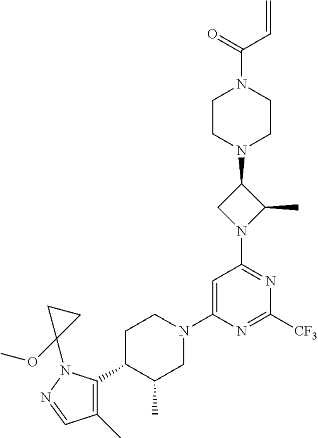 [SMILES: C=CC(=O)N1CCN([C@@H]2CN(c3cc(N4CC[C@@H](c5c(C)cnn5C5(OC)CC5)[C@@H](C)C4)nc(C(F)(F)F)n3)[C@@H]2C)CC1] | 1-(4-((2R,3R)-1-(6- ((3R,4R)-4-(1-(1- methoxycyclo- propyl)-4- methyl-1H- pyrazol-5-yl)-3- methyl-1- piperidinyl)-2- (trifluoromethyl)- 4-pyrimidinyl)-2- methyl-3- azetidinyl)-1- piperazinyl)-2- propen-1-one |
| 2-501 | 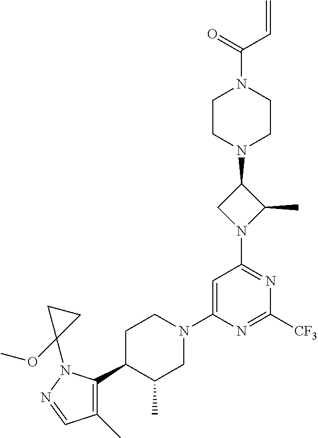 [SMILES: C=CC(=O)N1CCN([C@@H]2CN(c3cc(N4CC[C@H](c5c(C)cnn5C5(OC)CC5)[C@@H](C)C4)nc(C(F)(F)F)n3)[C@@H]2C)CC1] | 1-(4-((2R,3R)-1-(6- ((3R,4S)-4-(1-(1- methoxycyclo- propyl)-4- methyl-1H- pyrazol-5-yl)-3- nicthyl-1- piperidinyl)-2- (trifluoromethyl)- 4-pyrimidinyl)-2- methyl-3- azetidinyl)-1- piperazinyl)-2- propen-1-one | 2-505 | 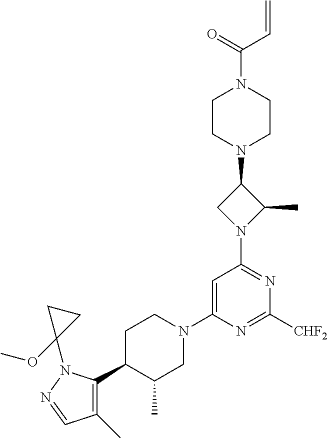 [SMILES: C=CC(=O)N1CCN([C@@H]2CN(c3cc(N4CC[C@H](c5c(C)cnn5C5(OC)CC5)[C@@H](C)C4)nc(C(F)F)n3)[C@@H]2C)CC1] | 1-(4-((2R,3R)-1-(2- (difluoromethyl)-6- ((3R,4S)-4-(1-(1- methoxycyclo- propyl)-4- methyl-1H- pyrazol-5-yl)-3- methyl-1- piperidinyl)-4- pyrimidinyl)-2- methyl-3- azetidinyl)-1- piperazinyl)-2- propen-1-one |
| 2-506 | 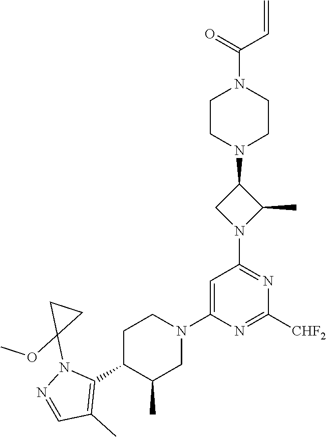 [SMILES: C=CC(=O)N1CCN([C@@H]2CN(c3cc(N4CC[C@@H](c5c(C)cnn5C5(OC)CC5)[C@H](C)C4)nc(C(F)F)n3)[C@@H]2C)CC1] | 1-(4-((2R,3R)-1-(2- (difluoromethyl)-6- ((3S,4R)-4-(1-(1- methoxycyclo- propyl)-4- methyl-1H- pyrazol-5-yl)-3- methyl-1- piperidinyl)-4- pyrimidinyl)-2- methyl-3- azetidinyl)-1- piperazinyl)-2- propen-1-one | 2-508 | 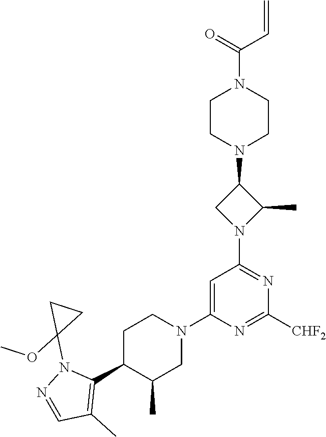 [SMILES: C=CC(=O)N1CCN([C@@H]2CN(c3cc(N4CC[C@H](c5c(C)cnn5C5(OC)CC5)[C@H](C)C4)nc(C(F)F)n3)[C@@H]2C)CC1] | 1-(4-((2R,3R)-1-(2- (difluoromethyl)-6- ((3,4S)-4-(1-(1- methoxycyclo- propyl)-4- methyl-1H- pyrazol-5-yl)-3- methyl-1- pipcridinyl)-4- pyrimidinyl)-2- methyl-3- azetidinyl)-1- piperazinyl)-2- propen-1-one |
| 2-507 | 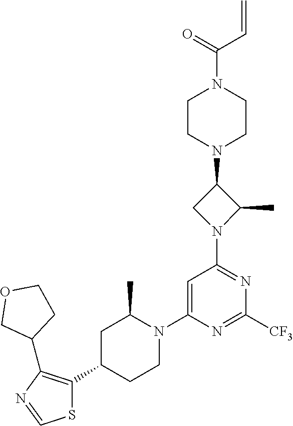 [SMILES: C=CC(=O)N1CCN([C@@H]2CN(c3cc(N4CC[C@H](c5scnc5C5CCOC5)C[C@H]4C)nc(C(F)(F)F)n3)[C@@H]2C)CC1] | 1-(4-((2R,3R)-2- methyl-1-(6- ((2R,4S)-2-methyl- 4-(4-(tetrahydro-3- furanyl)-1,3- thiazol-5-yl)-1- piperidinyl)-2- (trifluoromethyl)- 4-pyrimidiny])-3- azetidinyl)-1- piperazinyl)-2- propen-1-one | 2-509 | 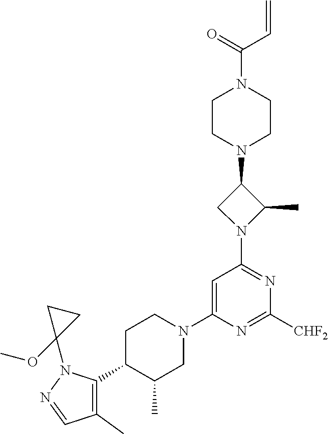 [SMILES: C=CC(=O)N1CCN([C@@H]2CN(c3cc(N4CC[C@@H](c5c(C)cnn5C5(OC)CC5)[C@@H](C)C4)nc(C(F)F)n3)[C@@H]2C)CC1] | 1-(4-((2R,3R)-1-(2- (difluoromethyl)-6- ((3R,4R)-4-(1-(1- methoxycyclo- propyl)-4-methyl- 1H-pyrazol-5-yl)- 3-methyl-1- piperidinyl)-4- pyrimidinyl)-2- methyl-3- azetidinyl)-]- piperazinyl)-2- propen-1-one |
| 2-507-1 | 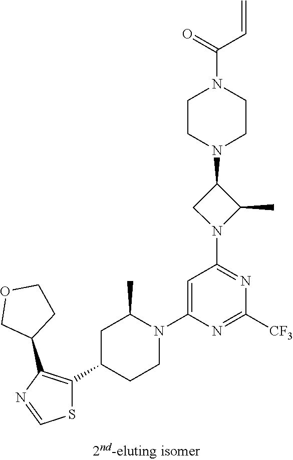 [SMILES: C=CC(=O)N1CCN([C@@H]2CN(c3cc(N4CC[C@H](c5scnc5[C@@H]5CCOC5)C[C@H]4C)nc(C(F)(F)F)n3)[C@@H]2C)CC1] | 1-(4-((2R,3R)-2- methyl-1-(6- ((2R,4S)-2-methyl- 4-(4-((35)- tetrahydro-3- furanyl)-1,3- thiazol-5-yl)-1- piperidinyl)-2- (trifluoromethyl)- 4-pyrimidinyl)-3- azetidinyl)-1- piperazinyl)-2- propen-1-one | 2-510 | 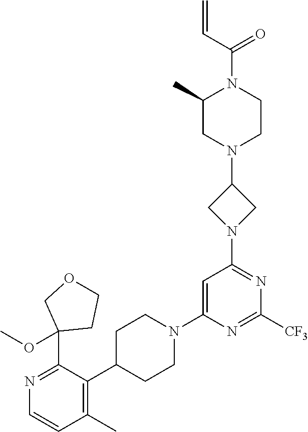 [SMILES: C=CC(=O)N1CCN(C2CN(c3cc(N4CCC(c5c(C)ccnc5C5(OC)CCOC5)CC4)nc(C(F)(F)F)n3)C2)C[C@H]1C] | 1-((2R)-4-(1-(6-(4- (2-(3- methoxytetrahydro -3-furany 1)-4- methyl-3- pyridinyl)-1- piperidinyl)-2- (trifluoromethyl)- 4-pyrimidinyl)-3- azetidinyl)-2- methyl-1- piperazinyl)-2- propen-1-one |
| 2-507-2 | 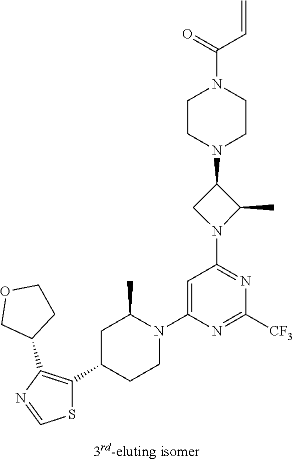 [SMILES: C=CC(=O)N1CCN([C@@H]2CN(c3cc(N4CC[C@H](c5scnc5[C@H]5CCOC5)C[C@H]4C)nc(C(F)(F)F)n3)[C@@H]2C)CC1] | 1-(4-((2R,3R)-2- methyl-1-(6- ((2R,4S)-2-methyl- 4-(4-((3R)- tetrahydro-3- foranyl)-1,3- thiazol-5-yl)-1- piperidinyl)-2- (trifluoromethyl)- 4-pyrintidinyl)-3- azetidinyl)-1- piperazinyl)-2- propen-1-one | 2-510-1 | 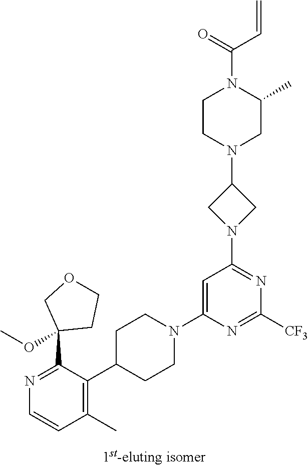 [SMILES: C=CC(=O)N1CCN(C2CN(c3cc(N4CCC(c5c(C)ccnc5[C@]5(OC)CCOC5)CC4)nc(C(F)(F)F)n3)C2)C[C@H]1C] | 1-((2R)-4-(1-(6-(4- (2-((3R)-3- methoxytetra- hydro- 3-furanyl)-4- methyl-3- pyridinyl)-1- piperidinyl)-2- (trifluoromethyl)- 4-pyrimidinyl)-3- azetidinyl)-2- methyl-1- piperazinyl)-2- propen-1-one |
| 2-510-2 | 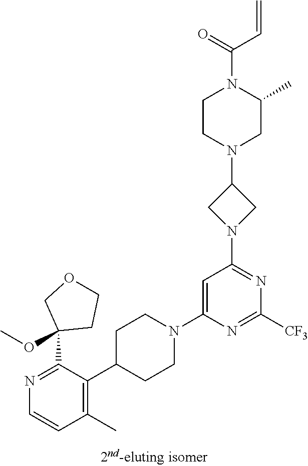 [SMILES: C=CC(=O)N1CCN(C2CN(c3cc(N4CCC(c5c(C)ccnc5[C@@]5(OC)CCOC5)CC4)nc(C(F)(F)F)n3)C2)C[C@H]1C] | 1-((2R)-4-(1-(6-(4- (2-((38)-3- methoxytetra- hydro-3-furanyl)- 4-methyl-3- pyridinyl)-1- piperidinyl)-2- (trifluoromcthyl)- 4-pyrimidinyl)-3- azetidinyl)-2- methyl-1- piperazinyl)-2- propen-1-one | 2-514 | 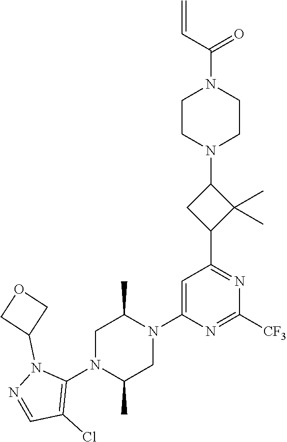 [SMILES: C=CC(=O)N1CCN(C2CC(c3cc(N4C[C@@H](C)N(c5c(Cl)cnn5C5COC5)C[C@H]4C)nc(C(F)(F)F)n3)C2(C)C)CC1] | 1-(4-(3-(6- ((2R,5R)-4-(4- chloro-1-(oxetan-3- yl)-1H-pyrazol-5- yl)-2.5- dimethylpiperazin- 1-yl)-2- (trifluoromethyl) pyrimidin-4-yl)- 2,2-dimethyl- cyclobutyl) piperazin-1- yl)prop-2-en-1-one |
| 2-511 | 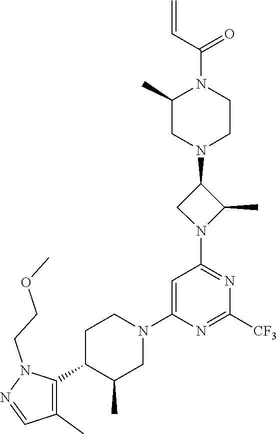 [SMILES: C=CC(=O)N1CCN([C@@H]2CN(c3cc(N4CC[C@@H](c5c(C)cnn5CCOC)[C@H](C)C4)nc(C(F)(F)F)n3)[C@@H]2C)C[C@H]1C] | 1-((2R)-4- ((2R,3R)-1-(6- ((3S,4R)-4-(1-(2- methoxyethyl)-4- methyl-1H- pyrazol-5-yl)-3- methyl-1- pipcridinyl)-2- (trifluoromethyl)- 4-pyrimidiny])-2- methyl-3- azetidinyl)-2- methyl-1- piperazinyl)-2- [symbol] | 2-514-1 | 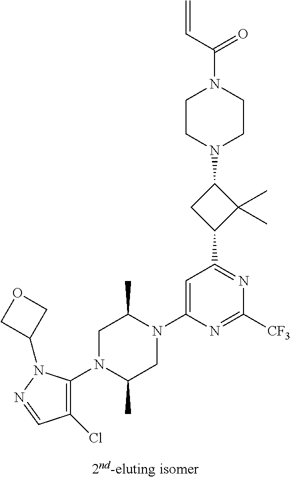 [SMILES: C=CC(=O)N1CCN([C@H]2C[C@@H](c3cc(N4C[C@@H](C)N(c5c(Cl)cnn5C5COC5)C[C@H]4C)nc(C(F)(F)F)n3)C2(C)C)CC1] | 1-(4-((1S,3R)-3-(6- ((2R,5R)-4-(4- chloro-1-(3- oxetanyl)-1H- pyrazol-5-yl)-2,5- dimethyl-1- piperazinyl)-2- (trifluoromethyl)- 4-pyrimidinyl)-2,2- dimethylcyclo- butyl)-1- piperazinyl)-2- propen-1-one |
| 2-512 | 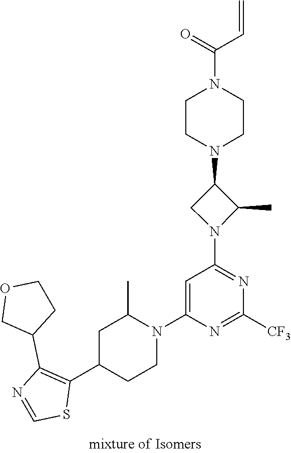 [SMILES: C=CC(=O)N1CCN([C@@H]2CN(c3cc(N4CCC(c5scnc5C5CCOC5)CC4C)nc(C(F)(F)F)n3)[C@@H]2C)CC1] | 1-(4-((2R.3R)-2- methyl-1-(6-(2- methyl-4-(4- (tetrahydro-3- furanyl)-1,3- thiazol-5-yl)-1- piperidinyl)-2- (trifluoromethyl)- 4-pyrimidiny])-3- azetidinyl)-1- piperazinyl)-2- propen-1-one | 2-515 | 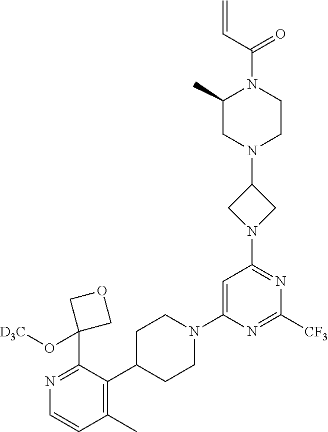 [SMILES: [2H]C([2H])([2H])OC1(c2nccc(C)c2C2CCN(c3cc(N4CC(N5CCN(C(=O)C=C)[C@H](C)C5)C4)nc(C(F)(F)F)n3)CC2)COC1] | (R)-1-(4-(1-(6-(4- (2-(3-(methoxy- d3)oxetan-3-y])-4- methylpyridin-3- yl)piperidin-1-yl)- 2.(trifluoromethyl) pyrimidin-4- yl)azetidin-3-yl)-2- methylpiperazin-1- yl)prop-2-en-1-one |
| 2-513 | 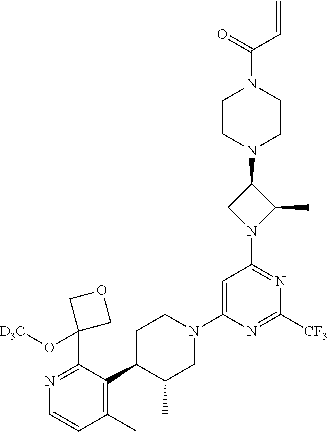 [SMILES: [2H]C([2H])([2H])OC1(c2nccc(C)c2[C@H]2CCN(c3cc(N4C[C@@H](N5CCN(C(=O)C=C)CC5)[C@H]4C)nc(C(F)(F)F)n3)C[C@@H]2C)COC1] | 1-(4-((2R,3R)-1-(6- ((3R,4S)-4-(2-(3- (methoxy- d₃)oxetan-3-yl)-4- methylpyridin-3- yl)-3- methylpiperidin-1- yl)-2- (trifluoromethyl) pyrimidin-4-yl)-2- methylazetidin-3- yl)piperazin-1- yl)prop-2-en-1-one | 2-516 | 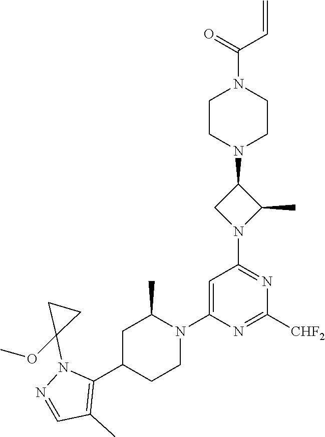 [SMILES: C=CC(=O)N1CCN([C@@H]2CN(c3cc(N4CCC(c5c(C)cnn5C5(OC)CC5)C[C@H]4C)nc(C(F)F)n3)[C@@H]2C)CC1] | 1-(4-((2R,3R)-1-(2- (difluoromethyl)-6- ((2R)-4-(1-(1- methoxycyclo- propyl)-4-mcthy]l- 1Hpyrazol-5-yl)-2- methylpiperidin-1- yl)pyrimidin-4-yl)- 2-methylazetidin- 3-yl)piperazin-1- yl)prop-2-en-1-one |
| 2-516-1 | 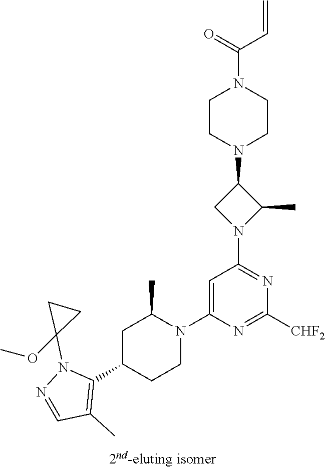 [SMILES: C=CC(=O)N1CCN([C@@H]2CN(c3cc(N4CC[C@H](c5c(C)cnn5C5(OC)CC5)C[C@H]4C)nc(C(F)F)n3)[C@@H]2C)CC1] | 1-(4-((2R,3R)-1-(2- (difluoromethyl)-6- ((2R,4S)-4-(1-(1- methoxycyclo- propyl)-4-methyl- 1H-pyrazol-5-yl)- 2-methyl-1- piperidinyl)-4- pyrimidinyl)-2- methyl-3- azetidinyl)-1- piperazinyl)-2- propen-1-one | 2-520 | 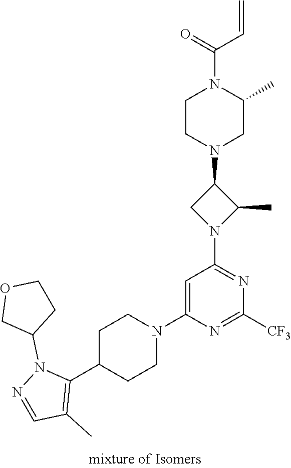 [SMILES: C=CC(=O)N1CCN([C@@H]2CN(c3cc(N4CCC(c5c(C)cnn5C5CCOC5)CC4)nc(C(F)(F)F)n3)[C@@H]2C)C[C@H]1C] | 1-((2R)-2-methyl- 4-((2R.3R)-2- methyl-1-(6-(4- (4-methyl-1- (tetrahydro-3- furanyl)-1H- pyrazol-5-yl)-1- pipcridinyl)-2- (trifluoromethyl)- 4-pyrimidinyl)-3. azetidinyl)-1- piperazinyl)-2- propen-1-one |
| 2-517 | 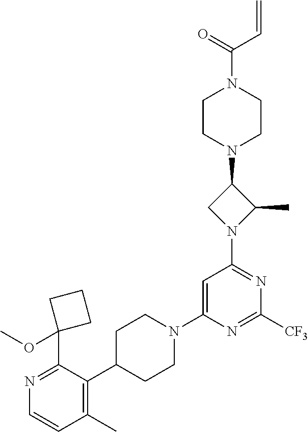 [SMILES: C=CC(=O)N1CCN([C@@H]2CN(c3cc(N4CCC(c5c(C)ccnc5C5(OC)CCC5)CC4)nc(C(F)(F)F)n3)[C@@H]2C)CC1] | 1-(4-((2R,3R)-1-(6- (4-(2-(1- methoxycyclo- butyl)-4-methyl-3- pyridinyl)-1- piperidinyl)-2- (trifluoromethyl)- 4-pyrimidinyl)-2- methyl-3- azetidinyl)-1- piperazinyl)-2- propen-1-one | 2-520-1 | 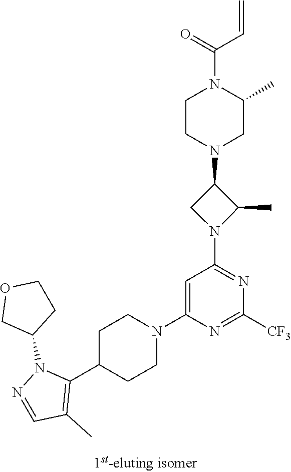 [SMILES: C=CC(=O)N1CCN([C@@H]2CN(c3cc(N4CCC(c5c(C)cnn5[C@H]5CCOC5)CC4)nc(C(F)(F)F)n3)[C@@H]2C)C[C@H]1C] | 1-((2R)-2-methyl- 4-((2R,3R)-2- methyl-1-(6-(4-(4- methyl-1-((3S)- tetrahydro-3- furanyl)-1H- pyrazol-5-y0)-1- piperidinyl)-2- (trifluoromethyl)- 4-pyrimidinyl)-3- azetidinyl)-1- piperazinyl)-2- propen-1-one |
| 2-518 | 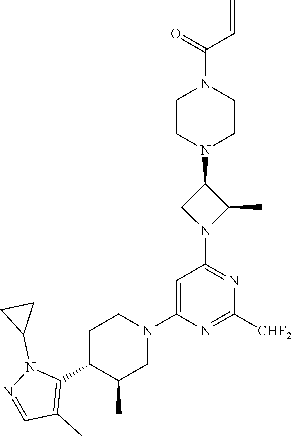 [SMILES: C=CC(=O)N1CCN([C@@H]2CN(c3cc(N4CC[C@@H](c5c(C)cnn5C5CC5)[C@H](C)C4)nc(C(F)F)n3)[C@@H]2C)CC1] | 1-(4-((2R,3R)-1-(6- ((3S,4R)-4-(1- cyclopropyl-4- methyl-TH- pyrazol-5-yl)-3- methyl-1- piperidiny])-2- (difluoromethyl)-4- pyrimidinyl)-2- methyl-3- azetidinyl)-1- piperazinyl)-2- propen-1-one | 2-520-2 | 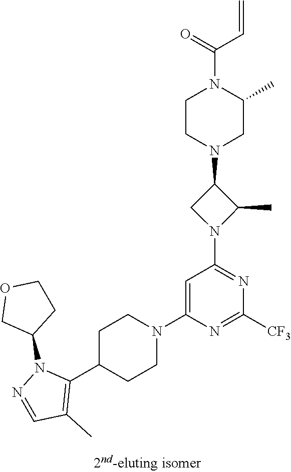 [SMILES: C=CC(=O)N1CCN([C@@H]2CN(c3cc(N4CCC(c5c(C)cnn5[C@@H]5CCOC5)CC4)nc(C(F)(F)F)n3)[C@@H]2C)C[C@H]1C] | 1-((2R)-2-methyl- 4-((2R,3R)-2- methyl-1-(6-(4-(4- methyl-1-((3R)- tetrahydro-3- furanyl)-1H- pyrazol-5-yl)-1- piperidinyl)-2- (trifluoromethyl)- 4-pyrimidinyl)-3- azetidinyl)-1- piperazinyl)-2- propen-1-one |
| 2-519 | 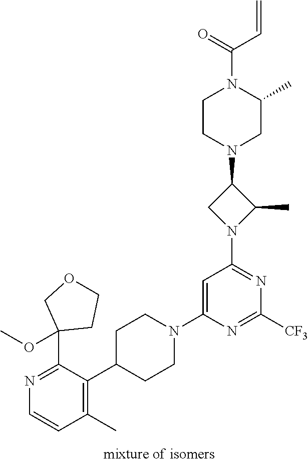 [SMILES: C=CC(=O)N1CCN([C@@H]2CN(c3cc(N4CCC(c5c(C)ccnc5C5(OC)CCOC5)CC4)nc(C(F)(F)F)n3)[C@@H]2C)C[C@H]1C] | 1-((2R)-4- ((2R,3R)-1-(6-(4- (2-(3- methoxytetra- hydro-3- furanyl)-4- metliyl-3- pyridinyl)-1- piperidinyl)-2- (trifluoromethyl)- 4-pyrimidinyl)-2- methyl-3- azetidinyl)-2- methyl-1- piperazinyl)-2- [symbol] | 2-521 | 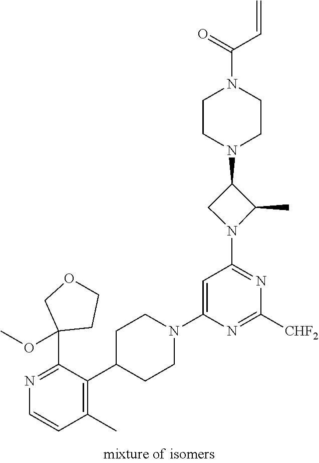 [SMILES: C=CC(=O)N1CCN([C@@H]2CN(c3cc(N4CCC(c5c(C)ccnc5C5(OC)CCOC5)CC4)nc(C(F)F)n3)[C@@H]2C)CC1] | 1-(4-((2R,3R)-1-(2- (difluoromethyl)-6- (4-(2-(3- methoxytetra- hydro-3-furanyl)- 4-methyl-3- pyridinyl)-1- piperidinyl)-4- pyrimidinyl)-2- methyl-3- azetidinyl)-1- piperazinyl)-2- propen-1-one |
| 2-522 | 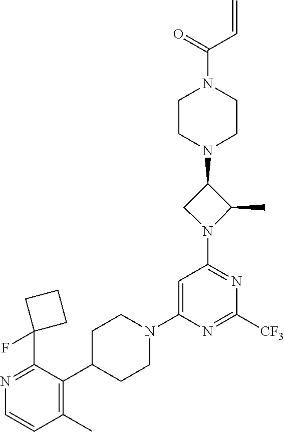 [SMILES: C=CC(=O)N1CCN([C@@H]2CN(c3cc(N4CCC(c5c(C)ccnc5C5(F)CCC5)CC4)nc(C(F)(F)F)n3)[C@@H]2C)CC1] | 1-(4-((2R,3R)-1-(6- (4-(2-(1- fluorocyclobutyl)- 4-methyl-3- pyridinyl)-1- piperidinyl)-2- (trifluoromethyl)- 4-pyrimidinyl)-2- methyl-3- azetidinyl)-1- piperazinyl)-2- propen-1-one | 2-526 | 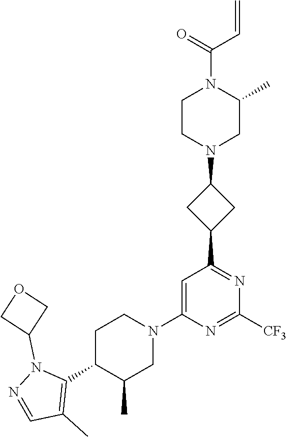 [SMILES: C=CC(=O)N1CCN([C@H]2C[C@@H](c3cc(N4CC[C@@H](c5c(C)cnn5C5COC5)[C@H](C)C4)nc(C(F)(F)F)n3)C2)C[C@H]1C] | I-((2R)-2-methyl- 4-(cis-3-(6- ((35,4R)-3-methyl- 4-(4-methyl-1-(3- oxetanyl)-1H- pyrazol-5-yl)-1- piperidinyl)-2- (trifluoromethyl)- 4-pyrimidinyl) cyclobutyl)-1- piperazinyl)-2- propen-1-one |
| 2-523 | 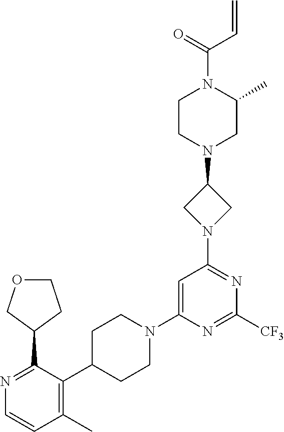 | 1-((2R)-2-methyl- 4-(1-(6-(4-(4- methyl-2-((3S)- tetrahydro-3- foranyl)-3- pytidinyl)-1- piperidinyl)-2- (trifluoromethyl)- 4-pyrimidinyl)-3- azetidinyl)-1- piperazinyl)-2- propen-1-one | 2-527 | 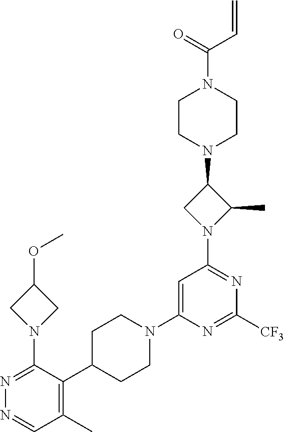 [SMILES: C=CC(=O)N1CCN([C@@H]2CN(c3cc(N4CCC(c5c(C)cnnc5N5CC(OC)C5)CC4)nc(C(F)(F)F)n3)[C@@H]2C)CC1] | 1-(4-((2R,3R)-1-(6- (4-(3-(3-methoxy- 1-azetidinyl)-5- methyl-4- pyridaziny])-1- piperidinyl)-2- (trifluoromethyl)- 4-pyrimidinyl)-2- methyl-3- azetidinyl)-1- piperazinyl)-2- propen-1-one |
| 2-524 | 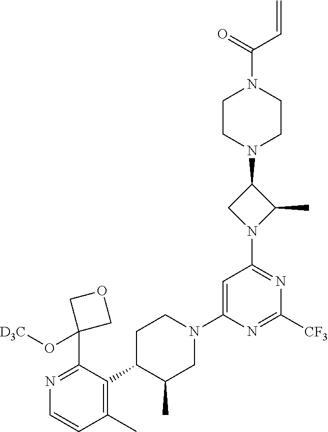 [SMILES: [2H]C([2H])([2H])OC1(c2nccc(C)c2[C@@H]2CCN(c3cc(N4C[C@@H](N5CCN(C(=O)C=C)CC5)[C@H]4C)nc(C(F)(F)F)n3)C[C@H]2C)COC1] | 1-(4-((2R,3R)-1-(6- ((3S,4R)-4-(2-(3- (methoxy- d₃)oxetan-3-yl)-4- methylpyridin-3- yl)-3- methylpiperidin-1- yl)-2- (trifluoromethyl) pyrimidin-4-yl)-2- methylazetidin-3- yl)piperazin-1- yl)prop-2-en-1-one | 2-528 | 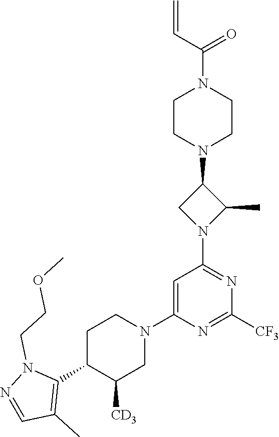 [SMILES: [2H]C([2H])([2H])[C@@H]1CN(c2cc(N3C[C@@H](N4CCN(C(=O)C=C)CC4)[C@H]3C)nc(C(F)(F)F)n2)CC[C@H]1c1c(C)cnn1CCOC] | 1-(4-((2R,3R)-1-(6- ((3S,4R)-4-(1-(2- methoxyethyl)-4- methyl-1H- pyrazol-5-yl)-3- (methyl- (3)piperichan-1-yl)- 2- (trifluoromethyl) pyrimidin-4-yl)-2- methylazetidin-3- yl)piperazin-1- yl)prop-2-en-1-one |
| 2-525 | 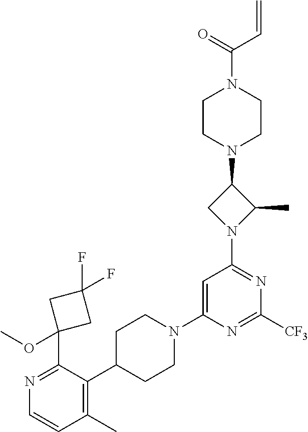 [SMILES: C=CC(=O)N1CCN([C@@H]2CN(c3cc(N4CCC(c5c(C)ccnc5C5(OC)CC(F)(F)C5)CC4)nc(C(F)(F)F)n3)[C@@H]2C)CC1] | 1-(4-((2R,3R)-1-(6- (4-(2-(3,3-difluoro- 1-methoxycyclo- butyl)-4-methyl-3- pyridinyl)-1- piperidinyl)-2- (trifluoromethyl)- 4-pyrimidinyl)-2- methyl-3- azetidinyl)-1- piperazinyl)-2- propen-1-one | 2-529 | 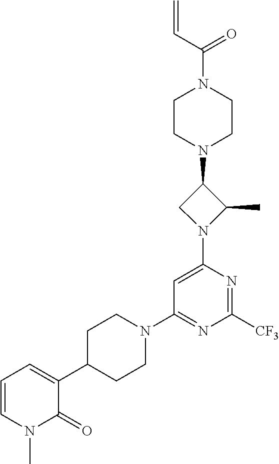 [SMILES: C=CC(=O)N1CCN([C@@H]2CN(c3cc(N4CCC(c5cccn(C)c5=O)CC4)nc(C(F)(F)F)n3)[C@@H]2C)CC1] | 1-methyl-3-(1-(6- ((2R.3R)-2-methyl- 3-(4-(2-propenoyl)- 1-piperazinyl)-1- azetidinyl)-2- (trifluoromethyl)- 4-pyrimidinyl)-4- piperidinyl)-2(1H)- pyridinone |
| 2-530 | 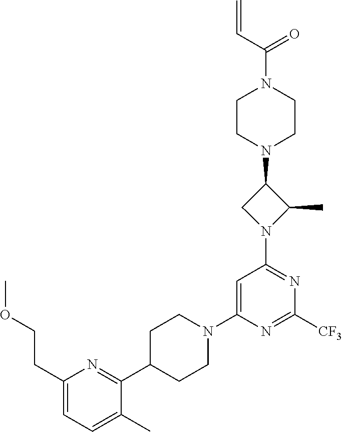 [SMILES: C=CC(=O)N1CCN([C@@H]2CN(c3cc(N4CCC(c5nc(CCOC)ccc5C)CC4)nc(C(F)(F)F)n3)[C@@H]2C)CC1] | 1-(4-((2R,3R)-1-(6- (4-(6-(2- methoxyethyl)-3- methyl-2- pyridinyl)-1- piperidinyl)-2- (trifluoromethyl)- 4-pyrimidiny])-2- methyl-3- azetidinyl)-1- piperazinyl)-2- propen-1-one | 2-532-1 | 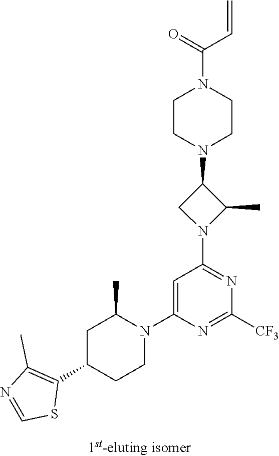 [SMILES: C=CC(=O)N1CCN([C@@H]2CN(c3cc(N4CC[C@H](c5scnc5C)C[C@H]4C)nc(C(F)(F)F)n3)[C@@H]2C)CC1] | 1-(4-((2R,3R)-2- methyl-1-(6- ((2R,4S)-2-methyl- 4-(4-methyl-1.3- thiazol-5-yl)-1- piperidinyl)-2- (trifluoromethyl)- 4-pyrimidinyl)-3- azetidinyl)-1- piperazinyl)-2- propen-1-one |
| 2-531 | 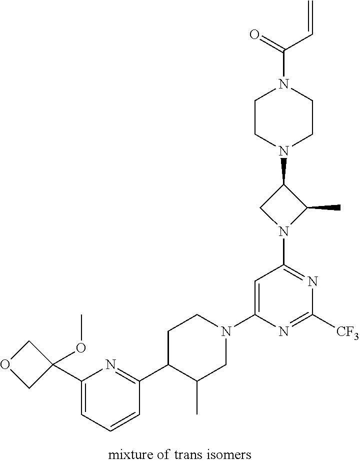 [SMILES: C=CC(=O)N1CCN([C@@H]2CN(c3cc(N4CCC(c5cccc(C6(OC)COC6)n5)C(C)C4)nc(C(F)(F)F)n3)[C@@H]2C)CC1] | 1-(4-((2R,3R)-1-(6- (4-(6-(3- methoxyoxetan-3- yl)pyridin-2-yl)-3- methylpiperidin-1- yl)-2- (trifluoromethyl) pyrimidin-4-yl)-2- methylazetidin-3- yl)piperazin-1- yl)prop-2-en-1-one | 2-533 | 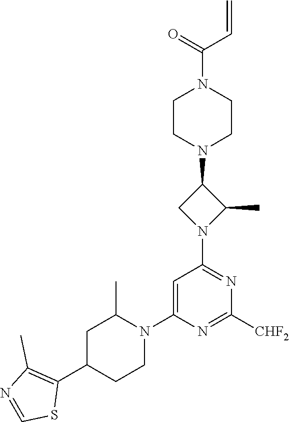 [SMILES: C=CC(=O)N1CCN([C@@H]2CN(c3cc(N4CCC(c5scnc5C)CC4C)nc(C(F)F)n3)[C@@H]2C)CC1] | 1-(4-((2R,3R)-1-(2- (difluoromethyl)-6- (2-methyl-4-(4- methylthiazol-5- yl)piperidin-1- yl)pyrimidin-4-yl)- 2-methylazetidin- 3-yl)piperazin-1- yl)prop-2-en-1-one |
| 2-531-1 | 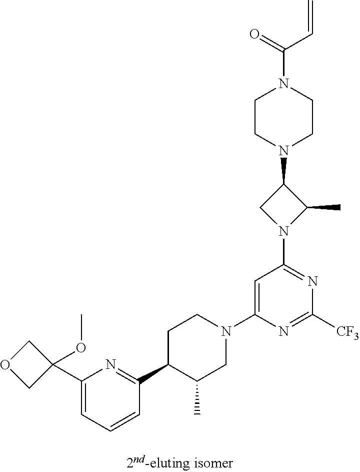 [SMILES: C=CC(=O)N1CCN([C@@H]2CN(c3cc(N4CC[C@H](c5cccc(C6(OC)COC6)n5)[C@@H](C)C4)nc(C(F)(F)F)n3)[C@@H]2C)CC1] | 1-(4-((2R,3R)-1-(6- ((3R,45)-4-(6-(3- methoxy-3- oxetanyl)-2- pyridinyl)-3- methyl-1- piperidiny])-2- (trifluoromethyl)- 4-pyrimidinyl)-2- methyl-3- azetidinyl)-1- piperazinyl)-2- propen-1-one | 2-533-1 | 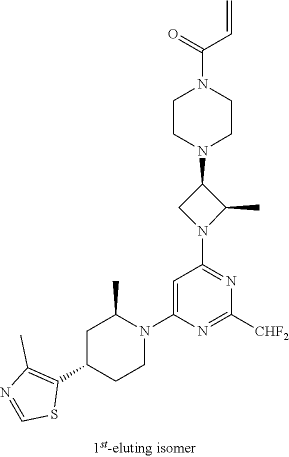 [SMILES: C=CC(=O)N1CCN([C@@H]2CN(c3cc(N4CC[C@H](c5scnc5C)C[C@H]4C)nc(C(F)F)n3)[C@@H]2C)CC1] | 1-(4-((2R,3R)-1-(2- (difluoromethyl)-6- ((2R,4S)-2-methyl- 4-(4-methyl-1,3- thiazol-5-yl)-1- piperidiny])-4- pyrimidinyl)-2- methyl-3- azetidinyl)-1- piperazinyl)-2- propen-1-one |
| 2-532 | 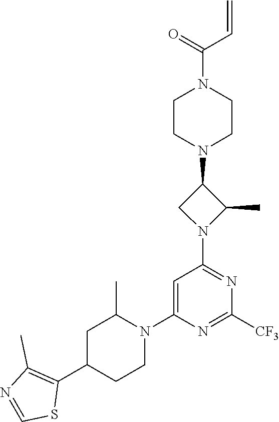 [SMILES: C=CC(=O)N1CCN([C@@H]2CN(c3cc(N4CCC(c5scnc5C)CC4C)nc(C(F)(F)F)n3)[C@@H]2C)CC1] | 1-(4-((2R,3R)-2- methyl-1-(6-(2- methyl-4-(4- methylthiazol-5. yl)piperidin-1-yl)- 2-(trifluoromethyl) pyrimidin-1- yl)azetidin-3- yl)piperazin-1- yl)prop-2-en-1-one | 2-534-1 | 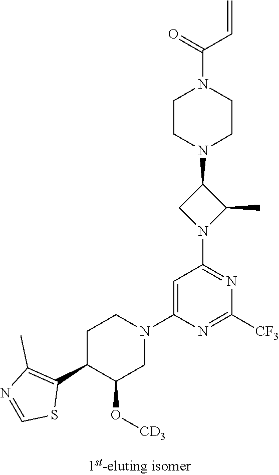 [SMILES: [2H]C([2H])([2H])O[C@@H]1CN(c2cc(N3C[C@@H](N4CCN(C(=O)C=C)CC4)[C@H]3C)nc(C(F)(F)F)n2)CC[C@@H]1c1scnc1C] | 1-(4-((2R,3R)-1-(6- ((3R,4R)-3- (methoxy-(3)-4-(4- methylthiazol-5- yl)piperidin-1-yl)- 2-(trifluoromethyl) pyrimidin-4-yl)-2- methylazetidin-3- yl)piperazin-1- yl)prop-2-en-1-one |
| 2-535 | 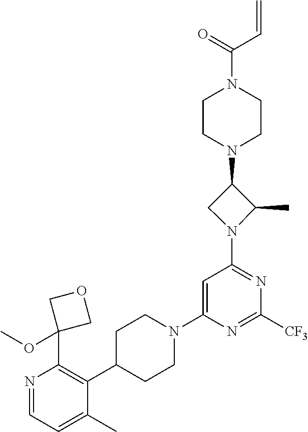 [SMILES: C=CC(=O)N1CCN([C@@H]2CN(c3cc(N4CCC(c5c(C)ccnc5C5(OC)COC5)CC4)nc(C(F)(F)F)n3)[C@@H]2C)CC1] | 1-(4-((2R,3R)-1-(6- (4-(3-(3-methoxy- 3-oxetanyl)-5- methyl-4- pyridinyl)-1- piperidinyl)-2- (trifluoromethyl)- 4-pyrimidinyl)-2- methyl-3- azetidinyl)-1- piperazinyl)-2- propen-1-one | 2-539 | 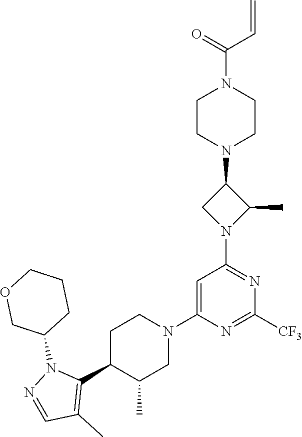 [SMILES: C=CC(=O)N1CCN([C@@H]2CN(c3cc(N4CC[C@H](c5c(C)cnn5[C@H]5CCCOC5)[C@@H](C)C4)nc(C(F)(F)F)n3)[C@@H]2C)CC1] | 1-(4-((2R,3R)-2- methyl-1-(6- ((3R,4S)-3-methyl- 4-(4-methyl-1- ((3S)-tetrahydro- 2H-pyran-3-yl)- 1H-pyrazol-5-yl)- 1-pipcridinyl)-2- (trifluoromethyl)- 4-pynimidinyl)-3- azetidinyl)-1- piperazinyl)-2- propen-1-one |
| 2-536 | 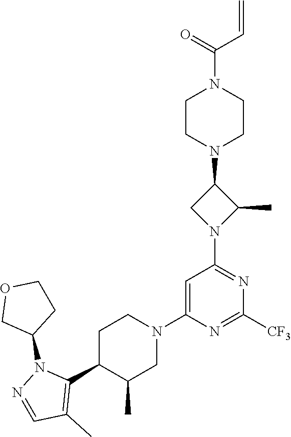 [SMILES: C=CC(=O)N1CCN([C@@H]2CN(c3cc(N4CC[C@H](c5c(C)cnn5[C@@H]5CCOC5)[C@H](C)C4)nc(C(F)(F)F)n3)[C@@H]2C)CC1] | 1-(4-((2R,3R)-2- methyl-1-(6- ((35,4S)-3-methyl- 4-(4-methyl-1- ((3R)-tetrahydro-3- furanyl)-1H- pyrazol-5-yl)-1- piperidinyl)-2- (trifluoromethyl)- 4-pyrimidinyl)-3- azetidinyl)-1- piperazinyl)-2- propen-1-one | 2-540 | 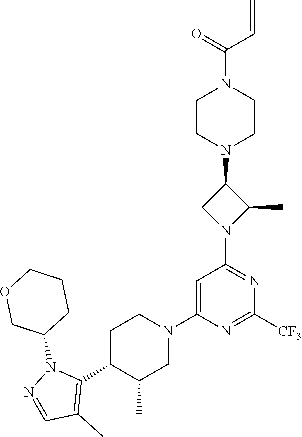 [SMILES: C=CC(=O)N1CCN([C@@H]2CN(c3cc(N4CC[C@@H](c5c(C)cnn5[C@H]5CCCOC5)[C@@H](C)C4)nc(C(F)(F)F)n3)[C@@H]2C)CC1] | 1-(4-((2R,3R)-2- methyl-1-(6- ((3R,4R)-3-methyl- 4-(4-methyl-1- ((3S)-tetrahydro- 2H-pyran-3-yl)- 1H-pyrazol-5-yl)- 1-piperidinyl)-2- (trifluoromethyl)- 4-pyrimidinyl)-3- azetidinyl)-]- piperazinyl)-2- propen-1-one |
| 2-537 | 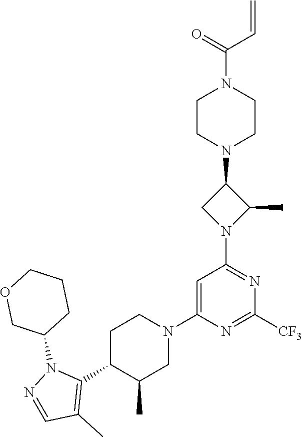 [SMILES: C=CC(=O)N1CCN([C@@H]2CN(c3cc(N4CC[C@@H](c5c(C)cnn5[C@H]5CCCOC5)[C@H](C)C4)nc(C(F)(F)F)n3)[C@@H]2C)CC1] | 1-(4-((2R,3R)-2- methyl-1-(6- ((3S,4R)-3-methyl- 4-(4-methyl-1- ((3S)-tetrahydro- 2H-pyran-3-yl)- 1H-pyrazol-5-yl)- 1-piperidinyl)-2- (trifluoromethyl)- 4-pyrimidinyl)-3- azetidinyl)-1- piperazinyl)-2- propen-1-one | 2-541 | 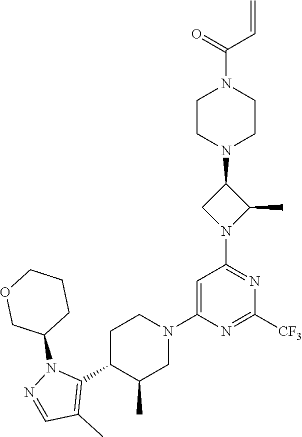 [SMILES: C=CC(=O)N1CCN([C@@H]2CN(c3cc(N4CC[C@@H](c5c(C)cnn5[C@@H]5CCCOC5)[C@H](C)C4)nc(C(F)(F)F)n3)[C@@H]2C)CC1] | 1-(4-((2R.3R)-2- methyl-1-(6- ((38,4R)-3-methyl- 4-(4-methyl-1- ((3R)-tetrahydro- 2H-pyran-3-yl)- 1H-pyrazol-5-yl)- 1-piperidinyl)-2- (trifluoromethyl)- 4-pyrimidinyl)-3- azetidinyl)-1- piperazinyl)-2- propen-1-one |
| 2-538 | 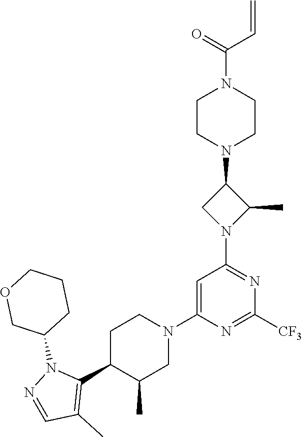 [SMILES: C=CC(=O)N1CCN([C@@H]2CN(c3cc(N4CC[C@H](c5c(C)cnn5[C@H]5CCCOC5)[C@H](C)C4)nc(C(F)(F)F)n3)[C@@H]2C)CC1] | 1-(4-((2R,3R)-2- methyl-1-(6- ((3S,4S)-3-methyl- 4-(4-methyl-1- ((3S)-tetrahydro- 2H-pyran-3-yl)- 1H-pyrazol-5-yl)- 1-piperidinyl)-2- (trifluoromethyl)- 4-pyrimidinyl)-3- azetidinyl)-1- piperazinyl)-2- propen-1-one | 2-542 | 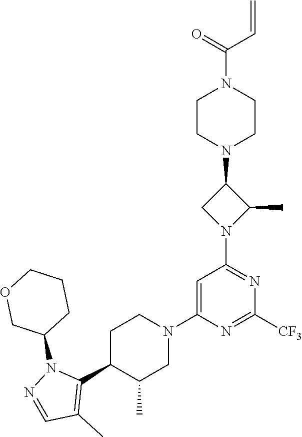 [SMILES: C=CC(=O)N1CCN([C@@H]2CN(c3cc(N4CC[C@H](c5c(C)cnn5[C@@H]5CCCOC5)[C@@H](C)C4)nc(C(F)(F)F)n3)[C@@H]2C)CC1] | 1-(4-((2R,3R)-2- methyl-1-(6- ((3R,4S)-3-methyl- 4-(4-methyl-1- ((3R)-tetrahydro- 2H-pyran-3-y])- 1H-pyrazol-5-yl)- 1-piperidinyl)-2- (trifluoromethyl)- 4-pyrintidinyl)-3- azetidinyl)-1- piperaziny])-2- propen-1-one |
| 2-543 | 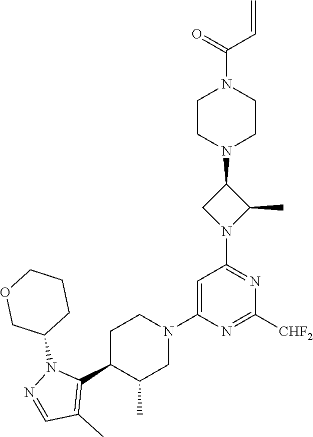 [SMILES: C=CC(=O)N1CCN([C@@H]2CN(c3cc(N4CC[C@H](c5c(C)cnn5[C@H]5CCCOC5)[C@@H](C)C4)nc(C(F)F)n3)[C@@H]2C)CC1] | 1-(4-((2R,3R)-1-(2- (difluoromethyl)-6- ((3R,4S)-3-methyl- 4-(4-methyl-1- ((3S)-tetrahydro- 2H-pyran-3-yl)- 1H-pyrazol-5-yl)- 1-piperidinyl)-4- pyrimidinyl)-2- methyl-3- azetidiny])-1- piperazinyl)-2- propen-1-one | 2-547 | 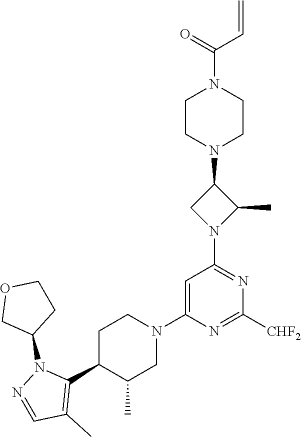 [SMILES: C=CC(=O)N1CCN([C@@H]2CN(c3cc(N4CC[C@H](c5c(C)cnn5[C@@H]5CCOC5)[C@@H](C)C4)nc(C(F)F)n3)[C@@H]2C)CC1] | 1-(4-((2R,3R)-1-(2- (difluoromethyl)-6- ((3R,4S)-3-methyl- 4-(4-methyl-1- ((3R)-tetrahydro-3- furanyl)-1H- pyrazol-5-yl)-1- pipcridinyl)-4- pyrimidinyl)-2- methyl-3- azetidinyl)-1- piperazinyl)-2- propen-1-one |
| 2-544 | 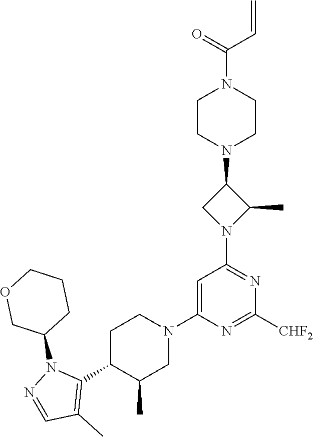 [SMILES: C=CC(=O)N1CCN([C@@H]2CN(c3cc(N4CC[C@@H](c5c(C)cnn5[C@@H]5CCCOC5)[C@H](C)C4)nc(C(F)F)n3)[C@@H]2C)CC1] | 1-(4-((2R,3R)-1-(2- (difluoromethyl)-6- ((3S,4R)-3-methyl- 4-(4-methyl-1- ((3R)-tetrahydro- 2H-pyran-3-yl)- 1H-pyrazol-5-yl)- 1-piperidinyl)-4- pyrimidinyl)-2- methyl-3- azetidinyl)-[- piperazinyl)-2- propen-1-one | 2-548 | 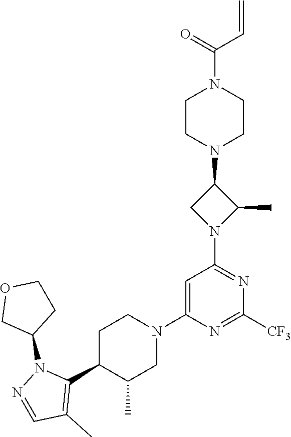 [SMILES: C=CC(=O)N1CCN([C@@H]2CN(c3cc(N4CC[C@H](c5c(C)cnn5[C@@H]5CCOC5)[C@@H](C)C4)nc(C(F)(F)F)n3)[C@@H]2C)CC1] | 1-(4-((2R,3R)-2- methyl-1-(6- ((3R,4S)-3-methyl- 4-(4-methyl-1- ((3R)-tetrahydro-3- furanyl)-1H- pyrazol-5-yl)-1- piperidinyl)-2- (trifluoromethyl)- 4-pyrimidinyl)-3- azetidinyl)-1- piperazinyl)-2- propen-1-one |
| 2-545 | 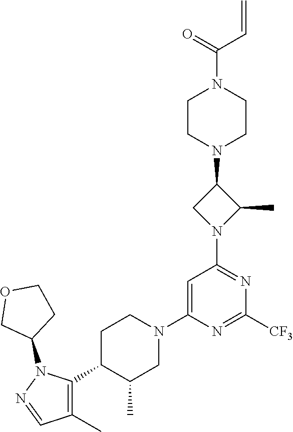 [SMILES: C=CC(=O)N1CCN([C@@H]2CN(c3cc(N4CC[C@@H](c5c(C)cnn5[C@@H]5CCOC5)[C@@H](C)C4)nc(C(F)(F)F)n3)[C@@H]2C)CC1] | 1-(4-((2R,3R)-2- methyl-1-(6- ((3R,4R)-3-methyl- 4-(4-methyl-1- ((3R)-tetrahydro-3- furanyl)-1H- pyrazol-5-yl)-1- piperidinyl)-2- (trifluoromethyl)- 4-pyrimidinyl)-3- azetidinyl)-1- piperazinyl)-2- propen-1-one | 2-549 | 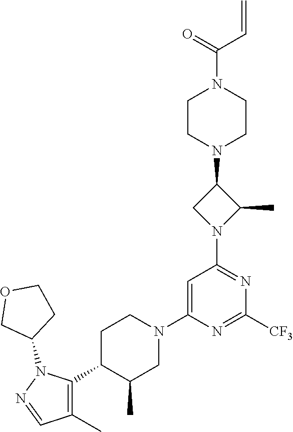 [SMILES: C=CC(=O)N1CCN([C@@H]2CN(c3cc(N4CC[C@@H](c5c(C)cnn5[C@H]5CCOC5)[C@H](C)C4)nc(C(F)(F)F)n3)[C@@H]2C)CC1] | 1-(4-((2R,3R)-2- methyl-1-(6- ((3S,4R)-3-methyl- 4-(4-methyl-1- ((3S)-tetrahydro-3- furanyl)-1H- pyrazol-5-yl)-1- piperidinyl)-2- (trifluoromethyl)- 4-pyrimidinyl)-3- azetidinyl)-1- piperazinyl)-2- propen-1-one |
| 2-546 | 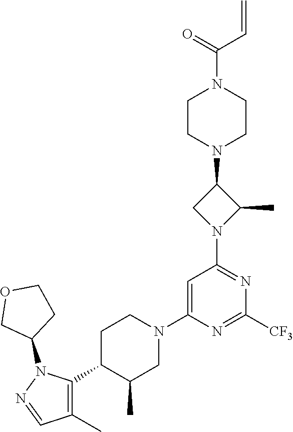 [SMILES: C=CC(=O)N1CCN([C@@H]2CN(c3cc(N4CC[C@@H](c5c(C)cnn5[C@@H]5CCOC5)[C@H](C)C4)nc(C(F)(F)F)n3)[C@@H]2C)CC1] | 1-(4-((2R,3R)-2- methyl-1-(6- ((3S,4R)-3-methyl- 4-(4-methyl-1- ((3R)-tetrahydro-3- foranyl)-1H- pyrazol-5-yl)-1- piperidinyl)-2- (trifluoromethyl)- 4-pyrimidinyl)-3- azetidinyl)-1- piperazinyl)-2- propen-1-one | 2-550 | 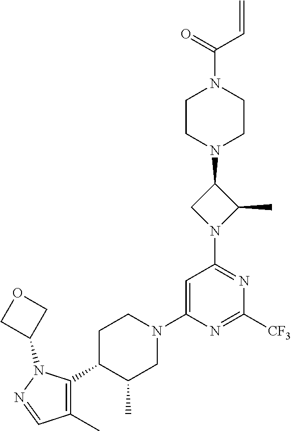 | 1-(4-((2R,3R)-2- methyl-1-(6- ((3R,4R)-3-methyl- 4-(4-methyl-1- ((3S)-tetrahydro-3- foranyl)-1H- pyrazol-5-yl)-1- piperidinyl)-2- (trifluoromethyl)- 4-pyrimidinyl)-3- azetidinyl)-1- piperazinyl)-2- propen-1-one |
| 2-551 | 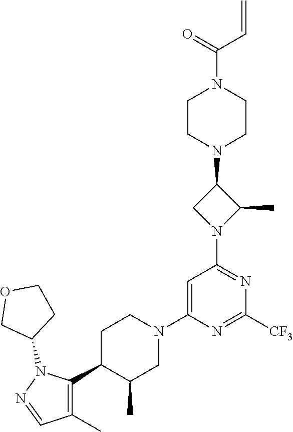 [SMILES: C=CC(=O)N1CCN([C@@H]2CN(c3cc(N4CC[C@H](c5c(C)cnn5[C@H]5CCOC5)[C@H](C)C4)nc(C(F)(F)F)n3)[C@@H]2C)CC1] | 1-(4-((2R,3R)-2- methyl-1-16- ((3S,4S)-3-methyl- 4-(4-methyl-1- ((3S)-tetrahydro-3- furanyl)-1H- pyrazol-5-yl)-1- piperidinyl)-2- (trifluoromethyl)- 4-pynimidinyl)-3- azetidinyl)-1- piperazinyl)-2- propen-1-one | 2-555 | 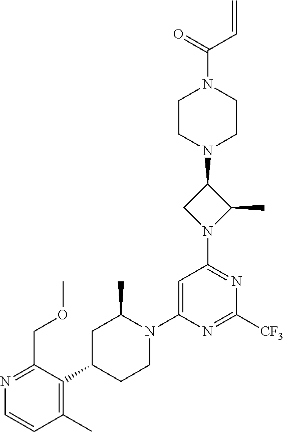 [SMILES: C=CC(=O)N1CCN([C@@H]2CN(c3cc(N4CC[C@H](c5c(C)ccnc5COC)C[C@H]4C)nc(C(F)(F)F)n3)[C@@H]2C)CC1] | 1-(4-((2R,3R)-1-(6- ((2R,4S)-4-(2- (methoxymethyl)- 4-methyl-3- pyridinyl)-2- methyl-1- piperidinyl)-2- (trifluoromethyl)- 4-pyrimidinyl)-2- methyl-3- azetidinyl)-1- piperazinyl)-2- propen-1-one |
| 2-552 | 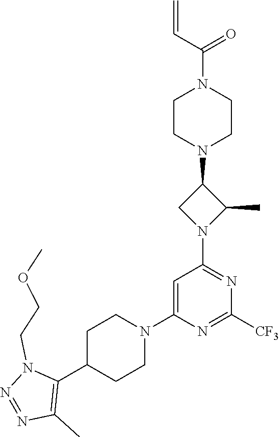 [SMILES: C=CC(=O)N1CCN([C@@H]2CN(c3cc(N4CCC(c5c(C)nnn5CCOC)CC4)nc(C(F)(F)F)n3)[C@@H]2C)CC1] | 1-(4-((2R,3R)-1-(6- (4-(1-(2- methoxyethyl)-4- methyl-1H-1,2,3- triazol-5-yl)-1- piperidiny])-2- (trifluoromethyl)- 4-pyrimidinyl)-2- methyl-3- azetidinyl)-1- piperazinyl)-2- propen-1-one | 2-556 | 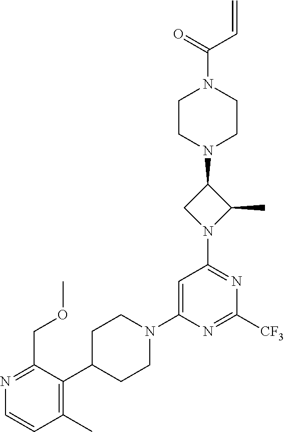 [SMILES: C=CC(=O)N1CCN([C@@H]2CN(c3cc(N4CCC(c5c(C)ccnc5COC)CC4)nc(C(F)(F)F)n3)[C@@H]2C)CC1] | 1-(4-((2R,3R)-1-(6- (4-(2- (methoxymethyl)- 4-methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-553 | 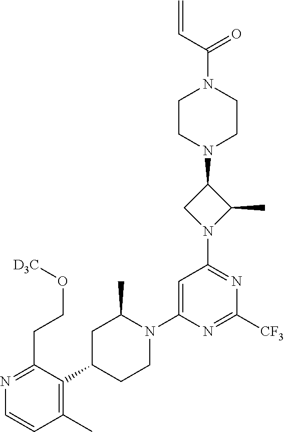 [SMILES: [2H]C([2H])([2H])OCCc1nccc(C)c1[C@H]1CCN(c2cc(N3C[C@@H](N4CCN(C(=O)C=C)CC4)[C@H]3C)nc(C(F)(F)F)n2)[C@H](C)C1] | 1-(4-((2R,3R)-1-(6- ((2R,4S)-4-(2-(2- (methoxy- d3)ethyl)-4- methylpyridin-3- yl)-2- methylpiperidin-1- yl)-2- (trifluoromethyl) pyrimidin-4-yl)-2- methylazetidin-3- yl)piperazin-1- yl)prop-2-en-1-one | 2-557 | 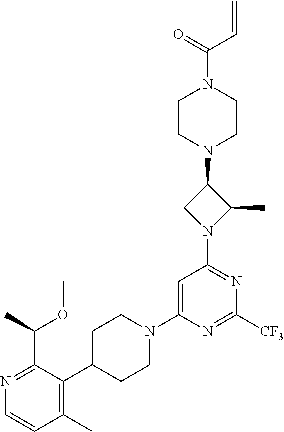 [SMILES: C=CC(=O)N1CCN([C@@H]2CN(c3cc(N4CCC(c5c(C)ccnc5[C@@H](C)OC)CC4)nc(C(F)(F)F)n3)[C@@H]2C)CC1] | 1-(4-((2R,3R)-1-(6- (4-(2-((1R)-1- methoxyethyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-554 | 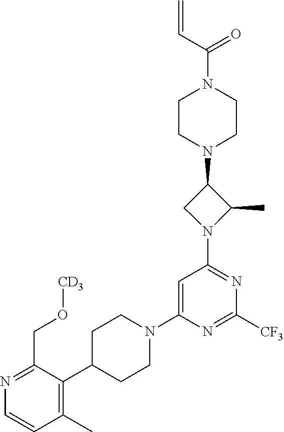 [SMILES: [2H]C([2H])([2H])OCc1nccc(C)c1C1CCN(c2cc(N3C[C@@H](N4CCN(C(=O)C=C)CC4)[C@H]3C)nc(C(F)(F)F)n2)CC1] | 1-(4-((2R,3R)-1-(6- (4-(2-((methoxy- d3)methyl)-4- methylpyridin-3- yl)piperidin-1-yl)- 2.(trifluoromethyl) pyrimidin-4-yl)-2- methylazetidin-3- yl)piperazin-1- yl)prop-2-en-1-one | 2-558 | 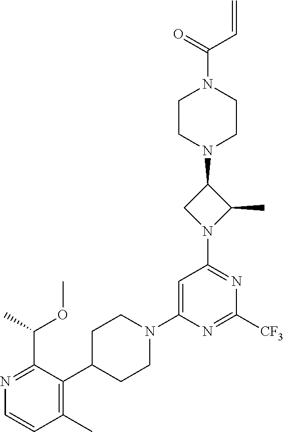 [SMILES: C=CC(=O)N1CCN([C@@H]2CN(c3cc(N4CCC(c5c(C)ccnc5[C@H](C)OC)CC4)nc(C(F)(F)F)n3)[C@@H]2C)CC1] | 1-(4-((2R,3R)-1-(6- (4-(2-((1S)-1- methoxyethyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-559 | 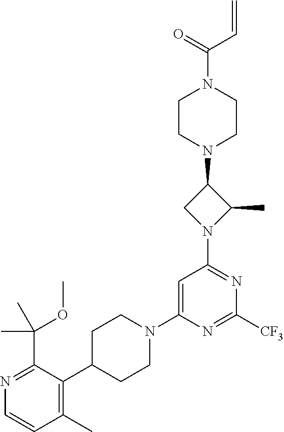 [SMILES: C=CC(=O)N1CCN([C@@H]2CN(c3cc(N4CCC(c5c(C)ccnc5C(C)(C)OC)CC4)nc(C(F)(F)F)n3)[C@@H]2C)CC1] | 1-(4-((2R,3R)-1-(6- (4-(2-(2-methoxy- 2-propanyl)-4- methyl-3- pyridinyl)-1- piperidinyl)-2- (trifluoromethyl)- 4-pyrimidiny])-2- methyl-3- azetidinyl)-1- piperazinyl)-2 propen-1-one | 2-563 | 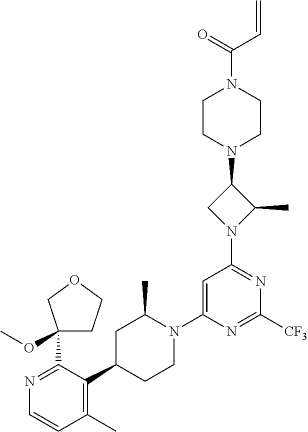 [SMILES: C=CC(=O)N1CCN([C@@H]2CN(c3cc(N4CC[C@@H](c5c(C)ccnc5[C@@]5(OC)CCOC5)C[C@H]4C)nc(C(F)(F)F)n3)[C@@H]2C)CC1] | 1-(4-((2R,3R)-1-(6- ((2R,4R)-4-(2- ((3S)-3- methoxytetra- hydro-3- furanyl)-4- methyl-3- pyridinyl)-2- methyl-1- piperidinyl)-2- (trifluoromethyl)- 4-pyrimidinyl)-2- methyl-3- azetidinyl)-1- piperaziny])-2- [symbol] |
| 2-560 | 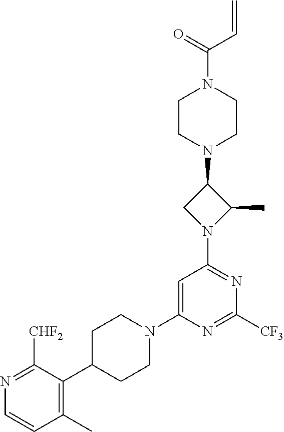 [SMILES: C=CC(=O)N1CCN([C@@H]2CN(c3cc(N4CCC(c5c(C)ccnc5C(F)F)CC4)nc(C(F)(F)F)n3)[C@@H]2C)CC1] | 1-(4-((2R,3R)-1-(6- (4-(2- (difluoromethyl)- 4-methyl-3- pyridinyl)-1- piperidiny])-2- (trifluoromethyl)- 4-pyrimidinyl)-2- methyl-3- azetidinyl)-1- piperazinyl)-2- propen-1-one | 2-564 | 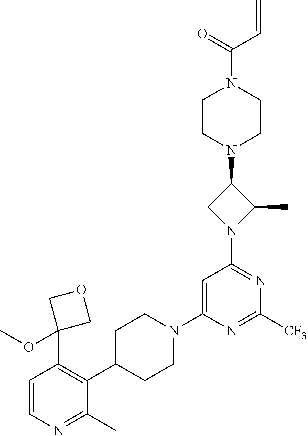 [SMILES: C=CC(=O)N1CCN([C@@H]2CN(c3cc(N4CCC(c5c(C6(OC)COC6)ccnc5C)CC4)nc(C(F)(F)F)n3)[C@@H]2C)CC1] | 1-(4-((2R,3R)-1-(6- (4-(4-(3-mcthoxy- 3-oxetanyl)-2- methyl-3- pyridinyl)-1- piperidiny])-2- (trifluoromethyl)- 4-pyrimidinyl)-2- methyl-3- azetidinyl)-1- piperazinyl)-2- propen-1-one |
| 2-561 | 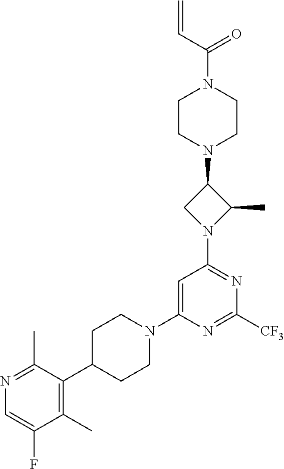 [SMILES: C=CC(=O)N1CCN([C@@H]2CN(c3cc(N4CCC(c5c(C)ncc(F)c5C)CC4)nc(C(F)(F)F)n3)[C@@H]2C)CC1] | 1-(4-((2R,3R)-1-(6- (4-(5-fluoro-2,4- dimethyl-3- pyridinyl)-1- piperidinyl)-2- (trifluoromethyl)- 4-pyrimidinyl)-2- methyl-3- azetidinyl)-1- piperazinyl)-2- propen-1-one | 2-565 | 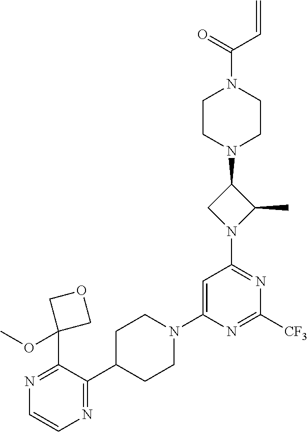 [SMILES: C=CC(=O)N1CCN([C@@H]2CN(c3cc(N4CCC(c5nccnc5C5(OC)COC5)CC4)nc(C(F)(F)F)n3)[C@@H]2C)CC1] | 1-(4-((2R,3R)-1-(6- (4-(3-(3-methoxy- 3-oxetanyl)-2- pyrazinyl)-1- piperidinyl)-2- (trifluoromethyl)- 4-pyrimidinyl)-2- methyl-3- azetidinyl)-1- piperazinyl)-2- propen-1-one |
| 2-562 | 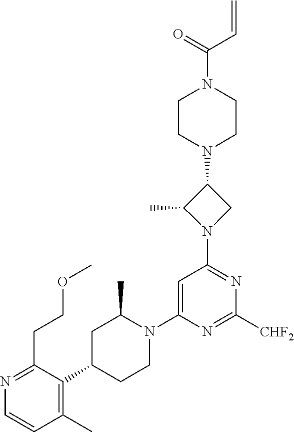 [SMILES: C=CC(=O)N1CCN([C@@H]2CN(c3cc(N4CC[C@H](c5c(C)ccnc5CCOC)C[C@H]4C)nc(C(F)F)n3)[C@@H]2C)CC1] | 1-(4-((2R,3R)-1-(2- (difluoromethyl)-6- ((2R.4S)-4-(2-(2- methoxyethyl)-4- methyl-3- pyridinyl)-2- methyl-1- piperidinyl)-4- pyrimidinyl)-2- methyl-3- azetidinyl)-1- piperazinyl)-2- propen-1-one | 2-566 | 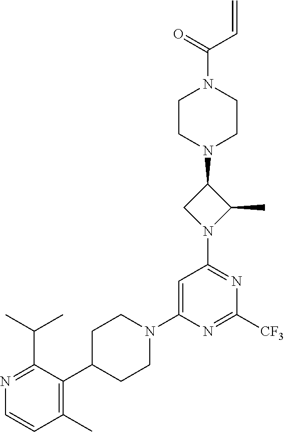 [SMILES: C=CC(=O)N1CCN([C@@H]2CN(c3cc(N4CCC(c5c(C)ccnc5C(C)C)CC4)nc(C(F)(F)F)n3)[C@@H]2C)CC1] | 1-(4-((2R,3R)-2- methyl-1-(6-(4-(4- methyl-2-(2- propanyl)-3- pyridinyl)-1- piperidinyl)-2- (trifluoromethyl)- 4-pyrimidinyl)-3- azetidinyl)-1- piperazinyl)-2- propen-t-one |
| 2-567 | 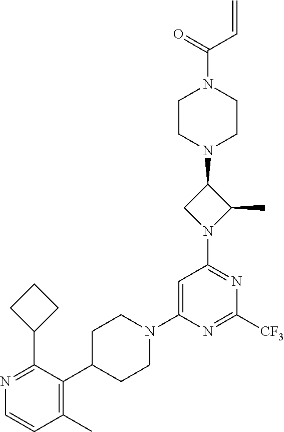 [SMILES: C=CC(=O)N1CCN([C@@H]2CN(c3cc(N4CCC(c5c(C)ccnc5C5CCC5)CC4)nc(C(F)(F)F)n3)[C@@H]2C)CC1] | 1-(4-((2R,3R)-1-(6- (4-(2-cyclobutyl-4- methyl-3- pyridinyl)-1- piperidiny])-2- (trifluoromethyl)- 4-pyrimidinyl)-2- methyl-3- azetidinyl)-1- piperazinyl)-2- propen-1-one | 2-571 | 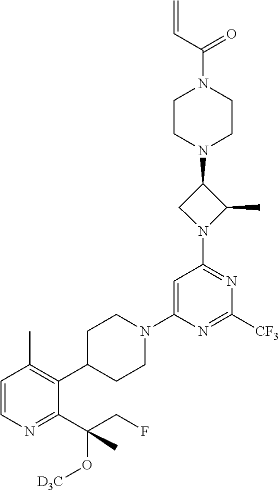 [SMILES: [2H]C([2H])([2H])O[C@@](C)(CF)c1nccc(C)c1C1CCN(c2cc(N3C[C@@H](N4CCN(C(=O)C=C)CC4)[C@H]3C)nc(C(F)(F)F)n2)CC1] | 1-(4-((2R,3R)-1-(6- (4-(2-((R)-1- fluoro-2-(methoxy- d₃)propan-2-yl)-4- methylpyridin-3- yl)piperidin-1-yl)- 2.(trifluoromethyl) pyrimidin-4-yl)-2- methylazetidin-3- ylpiperazin-1- yl)prop-2-en-1-one |
| 2-568 | 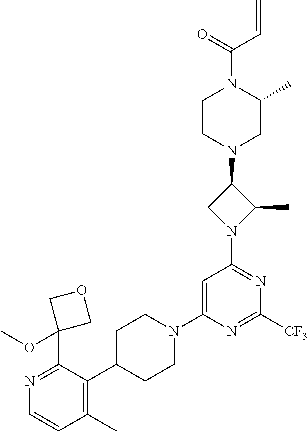 [SMILES: C=CC(=O)N1CCN([C@@H]2CN(c3cc(N4CCC(c5c(C)ccnc5C5(OC)COC5)CC4)nc(C(F)(F)F)n3)[C@@H]2C)C[C@H]1C] | 1-((2R)-4- ((2R,3R)-1-(6-(4- (2-(3-methoxy-3- oxetanyl)-4- methyl-3- pyridinyl)-1- piperidiny])-2- (trifluoromethyl)- 4-pyrimidinyl)-2- methyl-3- azetidinyl)-2- methyl-1- piperazinyl)-2- propen-1-one | 2-572 | 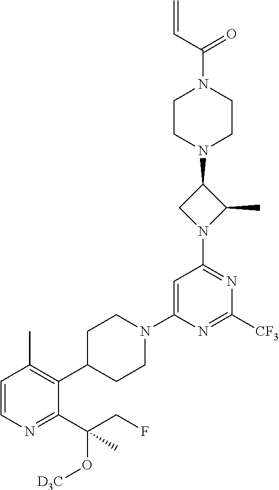 [SMILES: [2H]C([2H])([2H])O[C@](C)(CF)c1nccc(C)c1C1CCN(c2cc(N3C[C@@H](N4CCN(C(=O)C=C)CC4)[C@H]3C)nc(C(F)(F)F)n2)CC1] | 1-(4-((2R,3R)-1-(6- (4-(2-((S)-1-fluoro- 2-(methoxy- d₃)propan-2-yl)-4- methylpyridin-3- yl)piperidin-1-yl)- 2-(trifluoromethyl) pyrimidin-4-yl)-2- methylazetidin-3- yl)piperazin-1- yl)prop-2-en-1-one |
| 2-569 | 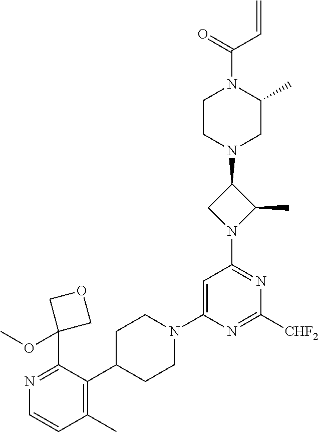 [SMILES: C=CC(=O)N1CCN([C@@H]2CN(c3cc(N4CCC(c5c(C)ccnc5C5(OC)COC5)CC4)nc(C(F)F)n3)[C@@H]2C)C[C@H]1C] | 1-((2R)-4- ((2R,3R)-1-(2- (difluoromethyl)-6- (4-(2-(3-methoxy- 3-oxetanyl)-4- methyl-3- pyridinyl)-1 piperidinyl)-4- pyrimidinyl)-2- methyl-3- azetidinyl)-2- methyl-1- piperazinyl)-2- propen-1-one | 2-573 | 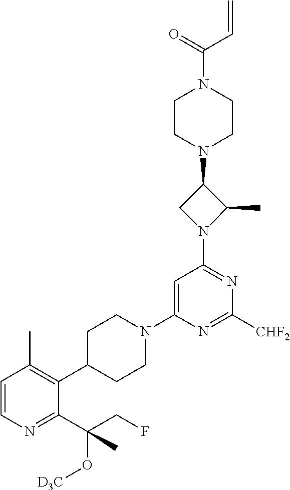 [SMILES: [2H]C([2H])([2H])O[C@@](C)(CF)c1nccc(C)c1C1CCN(c2cc(N3C[C@@H](N4CCN(C(=O)C=C)CC4)[C@H]3C)nc(C(F)F)n2)CC1] | 1-(4-((2R,3R)-1-(2- (difluoromethyl)-6- (4-(2-((R)-1- fluoro-2-(methoxy- d₃)propan-2-yl)-4- methylpyridin-3- yl)piperidin-1- yl)pyrimidin-4-yl)- 2-methylazetidin- 3-yl)piperazin-1- yl)prop-2-en-1-one |
| 2-570 | 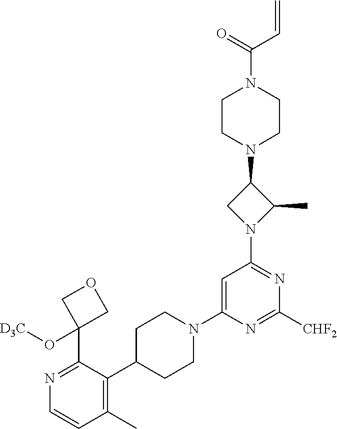 [SMILES: [2H]C([2H])([2H])OC1(c2nccc(C)c2C2CCN(c3cc(N4C[C@@H](N5CCN(C(=O)C=C)CC5)[C@H]4C)nc(C(F)F)n3)CC2)COC1] | 1-(4-((2R,3R)-1-(2- (difluoromethyl)-6- (4-(2-(3-(methoxy- d3)oxetan-3-yl)-4- methylpyridin-3- yl)piperidin-1- yl)pyrimidin-4-yl)- 2-methylazetidin- 3-yl)piperazin-1- yl)prop-2-en-1-one | 2-574 | 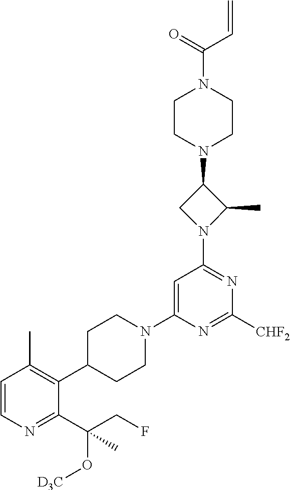 [SMILES: [2H]C([2H])([2H])O[C@](C)(CF)c1nccc(C)c1C1CCN(c2cc(N3C[C@@H](N4CCN(C(=O)C=C)CC4)[C@H]3C)nc(C(F)F)n2)CC1] | 1-(4-((2R,3R)-1-(2- (difluoromethyl)-6- (4-(2-((S)-1-fluoro- 2-(methoxy- d₃)propan-2-yl)-4- methylpyridin-3- yl)piperidin-1- yl)pyrimidin-4-yl)- 2-methylazetidin- 3-yl)piperazin-1- yl)prop-2-en-1-one |
| 2-575 | 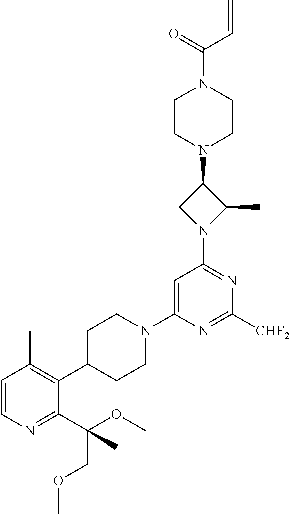 [SMILES: C=CC(=O)N1CCN([C@@H]2CN(c3cc(N4CCC(c5c(C)ccnc5[C@@](C)(COC)OC)CC4)nc(C(F)F)n3)[C@@H]2C)CC1] | 1-(4-((2R,3R)-1-(2- (difluoromethyl)-6- (4-(2-((2S)-1,2- dimethoxy-2- propanyl)-4- methyl-3- pyridinyl)-1- piperidinyl)-4- pyrimidinyl)-2- methyl-3- azetidinyl)-1- piperazinyl)-2- propen-1-one | 2-579 | 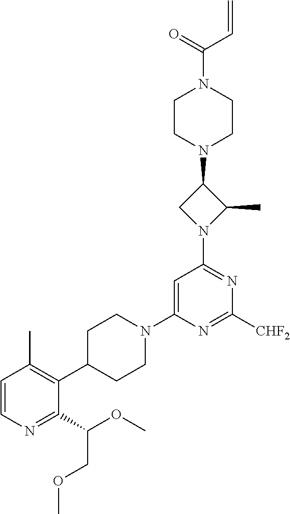 [SMILES: C=CC(=O)N1CCN([C@@H]2CN(c3cc(N4CCC(c5c(C)ccnc5[C@@H](COC)OC)CC4)nc(C(F)F)n3)[C@@H]2C)CC1] | 1-(4-((2R,3R)-1-(2- (difluoromethyl)-6- (4-(2-((1S)-1.2- dimethoxyethyl)-4- methyl-3- pyridinyl)-1- piperidinyl)-4- pyrimidiny])-2- methyl-3- azetidinyl)-1- piperazinyl)-2- propen-1-one |
| 2-576 | 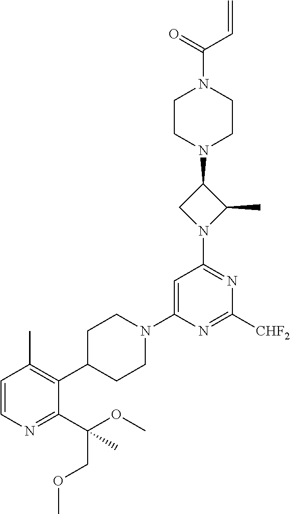 [SMILES: C=CC(=O)N1CCN([C@@H]2CN(c3cc(N4CCC(c5c(C)ccnc5[C@](C)(COC)OC)CC4)nc(C(F)F)n3)[C@@H]2C)CC1] | 1-(4-((2R,3R)-1-(2- (difluoromethyl)-6- (4-(2-((2R)-1,2- dimethoxy-2- propanyl)-4- methyl-3- pyridinyl)-1- piperidinyl)-4- pyrimidinyl)-2- methyl-3- azetidinyl)-1- piperazinyl)-2- propen-1-one | 2-580 | 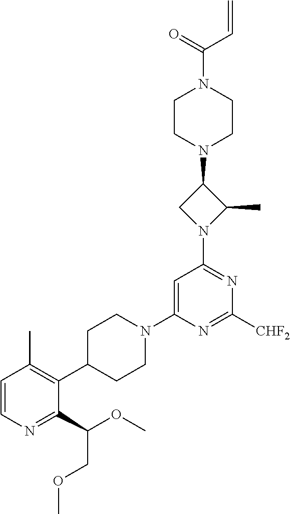 [SMILES: C=CC(=O)N1CCN([C@@H]2CN(c3cc(N4CCC(c5c(C)ccnc5[C@H](COC)OC)CC4)nc(C(F)F)n3)[C@@H]2C)CC1] | 1-(4-((2R,3R)-1-(2- (difluoromcthyl)-6- (4-(2-((1R)-1,2- dimethoxyethyl)-4- methyl-3- pyridinyl)-1- piperidinyl)-4- pyrimidinyl)-2- methyl-3- azetidinyl)-1- piperazinyl)-2- propen-1-one |
| 2-577 | 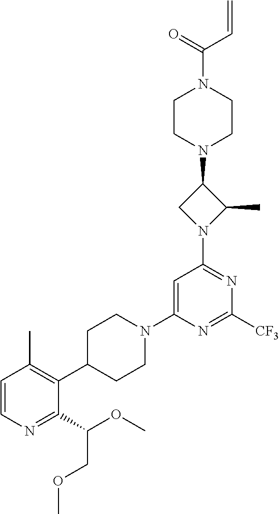 [SMILES: C=CC(=O)N1CCN([C@@H]2CN(c3cc(N4CCC(c5c(C)ccnc5[C@@H](COC)OC)CC4)nc(C(F)(F)F)n3)[C@@H]2C)CC1] | 1-(4-((2R,3R)-1-(6- (4-(2-((1S)-1,2- dimethoxyethyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one | 2-581 | 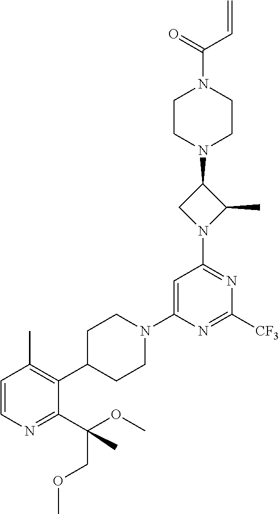 [SMILES: C=CC(=O)N1CCN([C@@H]2CN(c3cc(N4CCC(c5c(C)ccnc5[C@@](C)(COC)OC)CC4)nc(C(F)(F)F)n3)[C@@H]2C)CC1] | 1-(4-((2R,3R)-1-(6- (4-(2-((2S)-1,2- dimethoxy-2- propanyl)-4- methyl-3- pyridinyl)-1- piperidiny])-2- (trifluoromethyl)- 4-pyrimidinyl)-2- methyl-3- azetidinyl)-1- piperazinyl)-2- propen-1-one |
| 2-578 | 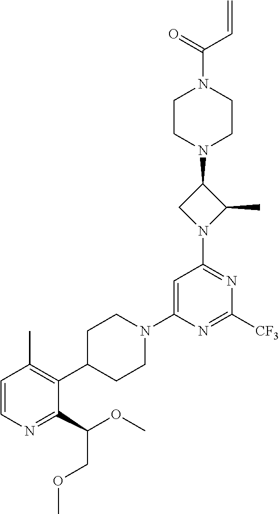 [SMILES: C=CC(=O)N1CCN([C@@H]2CN(c3cc(N4CCC(c5c(C)ccnc5[C@H](COC)OC)CC4)nc(C(F)(F)F)n3)[C@@H]2C)CC1] | 1-(4-((2R,3R)-1-(6- (4-(2-((1R)-1,2- dimethoxyethyl)-1- methyl-3- pyridinyl)-1- piperidinyl)-2- (trifluoromethyl)- 4-pyrimidinyl)-2- methyl-3- azetidiny])-1- piperazinyl)-2- propen-1-one | 2-582 | 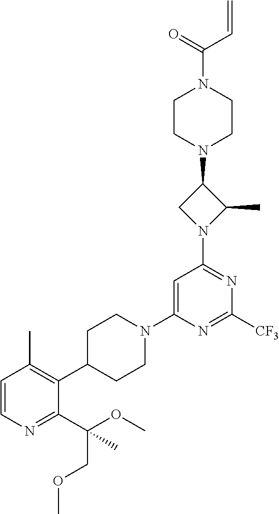 [SMILES: C=CC(=O)N1CCN([C@@H]2CN(c3cc(N4CCC(c5c(C)ccnc5[C@](C)(COC)OC)CC4)nc(C(F)(F)F)n3)[C@@H]2C)CC1] | 1-(4-((2R,3R)-1-(6- (4-(2-((2R)-1,2- dimethoxy-2- propanyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-583 | 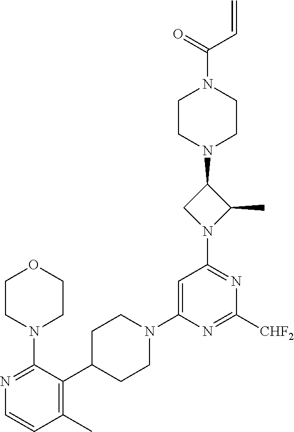 [SMILES: C=CC(=O)N1CCN([C@@H]2CN(c3cc(N4CCC(c5c(C)ccnc5N5CCOCC5)CC4)nc(C(F)F)n3)[C@@H]2C)CC1] | 1-(4-((2R,3R)-1-(2- (difluoromethyl)-6- (4-(4-methyl-2-(4- morpholinyl)-3- pyridinyl)-1- piperidinyl)-4- pyrimidinyl)-2- methyl-3- azetidinyl)-1- piperazinyl)-2- propen-1-one | 2-588 | 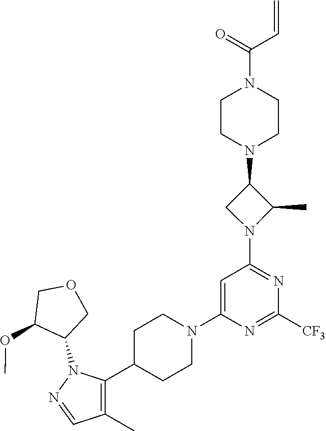 [SMILES: C=CC(=O)N1CCN([C@@H]2CN(c3cc(N4CCC(c5c(C)cnn5[C@H]5COC[C@@H]5OC)CC4)nc(C(F)(F)F)n3)[C@@H]2C)CC1] | 1-(4-((2R,3R)-1-(6- (4-(1-((3S.4R)-4- methoxytetrahydro- 3-furanyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one |
| 2-584 | 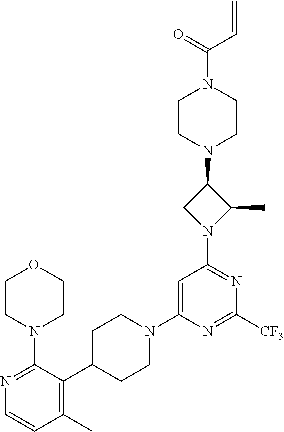 [SMILES: C=CC(=O)N1CCN([C@@H]2CN(c3cc(N4CCC(c5c(C)ccnc5N5CCOCC5)CC4)nc(C(F)(F)F)n3)[C@@H]2C)CC1] | 1-(4-((2R,3R)-2- methyl-1-(6-(4-(4- methyl-2-(4- morpholinyl)-3- pyridinyl)-1- piperidinyl)-2- (trifluoromethyl)- 4-pyrintidinyl)-3- azetidinyl)-1- piperazinyl)-2- propen-1-one | 2-589 | 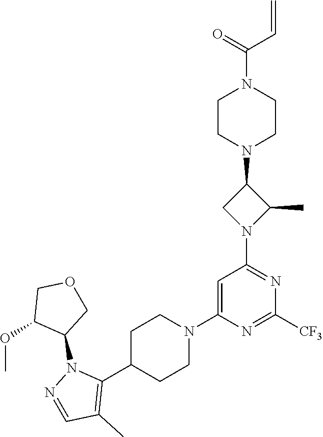 [SMILES: C=CC(=O)N1CCN([C@@H]2CN(c3cc(N4CCC(c5c(C)cnn5[C@@H]5COC[C@H]5OC)CC4)nc(C(F)(F)F)n3)[C@@H]2C)CC1] | 1-(4-((2R,3R)-1-(6- (4-(1-((3R,4S)-4- methoxytetrahydro- 3-furanyl)-4- methyl-1H- pyrazol-5-yl)-1- piperidinyl)-2- (trifluoromethyl)- 4-pyrintidinyl)-2- methyl-3- azetidinyl)-1- piperazinyl)-2- propen-1-one |
| 2-585 | 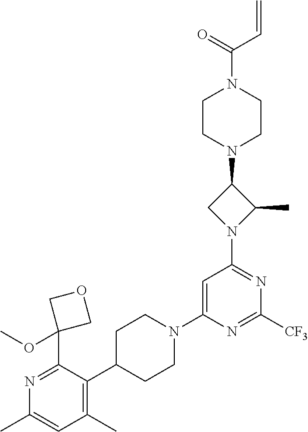 [SMILES: C=CC(=O)N1CCN([C@@H]2CN(c3cc(N4CCC(c5c(C)cc(C)nc5C5(OC)COC5)CC4)nc(C(F)(F)F)n3)[C@@H]2C)CC1] | 1-(4-((2R,3R)-1-(6- (4-(2-(3-methoxy- 3-oxetanyl)-4,6- dimethyl-3- pyridinyl)-1- piperidinyl)-2- (trifluoromethyl)- 4-pyrimidinyl)-2- methyl-3- azetidinyl)-1- piperazinyl)-2- propen-1-one | 2-590 | 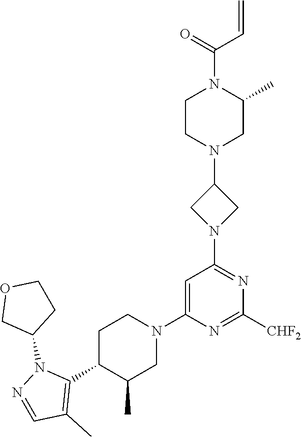 [SMILES: C=CC(=O)N1CCN(C2CN(c3cc(N4CC[C@@H](c5c(C)cnn5[C@H]5CCOC5)[C@H](C)C4)nc(C(F)F)n3)C2)C[C@H]1C] | 1-((2R)-4-(1-(2- (difluoromethyl)-6- ((3S,4R)-3-methyl- 4-(4-methyl-1- ((3S)-tetrahydro-3- furanyl)-1H- pyrazol-5-yl)-1- piperidinyl)-4- pyrimidinyl)-3- azetidinyl)-2- methyl-1- piperazinyl)-2- propen-1-one |
| 2-586 | 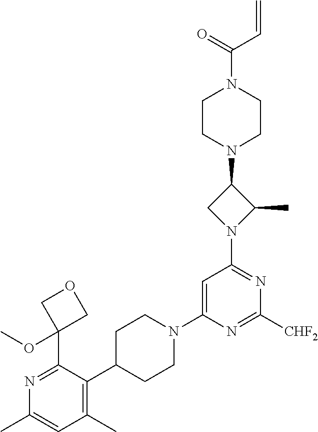 [SMILES: C=CC(=O)N1CCN([C@@H]2CN(c3cc(N4CCC(c5c(C)cc(C)nc5C5(OC)COC5)CC4)nc(C(F)F)n3)[C@@H]2C)CC1] | 1-(4-((2R,3R)-1-(2- (difluoromethyl)-6- (4-(2-(3-methoxy- 3-oxetanyl)-4,6- dimethyl-3- pyridinyl)-1- piperidiny])-4- pyrimidinyl)-2- methyl-3- azetidiny])-1- piperazinyl)-2- propen-1-one | 2-591 | 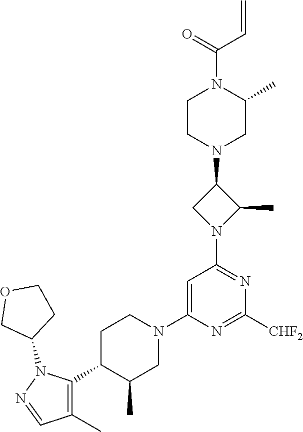 [SMILES: C=CC(=O)N1CCN([C@@H]2CN(c3cc(N4CC[C@@H](c5c(C)cnn5[C@H]5CCOC5)[C@H](C)C4)nc(C(F)F)n3)[C@@H]2C)C[C@H]1C] | 1-((2R)-4- ((2R,3R)-1-(2- (difluoromethy])-6- ((3S,4R)-3-methyl- 4-(4-methyl-1- ((3S)-tetrahydro-3- furanyl)-1H- pyrazol-5-yl)-1- piperidinyl)-4- pyrimidinyl)-2- methyl-3- azetidinyl)-2- methyl-1- piperazinyl)-2- [symbol] |
| 2-592 | 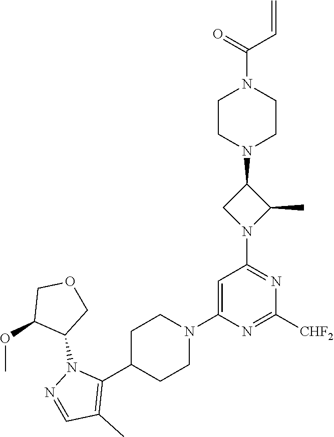 [SMILES: C=CC(=O)N1CCN([C@@H]2CN(c3cc(N4CCC(c5c(C)cnn5[C@H]5COC[C@@H]5OC)CC4)nc(C(F)F)n3)[C@@H]2C)CC1] | 1-(4-((2R,3R)-1-(2- (difluoromethyl)-6- (4-(1-((3S,4R)-4- methoxytetrahydro- 3-furanyl)-4- methyl-1H- pyrazol-5-yl)-1- piperidinyl)-4- pyrimidinyl)-2- methyl-3- azetidinyl)-1- piperazinyl)-2- propen-1-one | 2-596 | 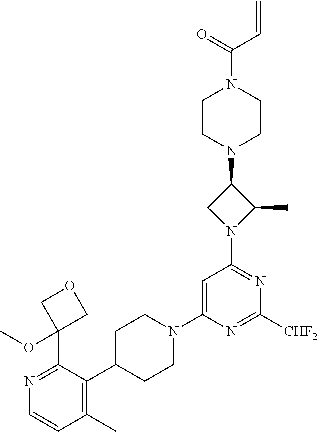 [SMILES: C=CC(=O)N1CCN([C@@H]2CN(c3cc(N4CCC(c5c(C)ccnc5C5(OC)COC5)CC4)nc(C(F)F)n3)[C@@H]2C)CC1] | 1-(4-((2R,3R)-1-(2- (difluoromethyl)-6- (4-(2-(3-methoxy- 3-oxetanyl)-4- methyl-3- pyridinyl)-1- piperidinyl)-4- pyrimidinyl)-2- methyl-3- azetidinyl)-1- piperazinyl)-2- propen-1-one |
| 2-593 | 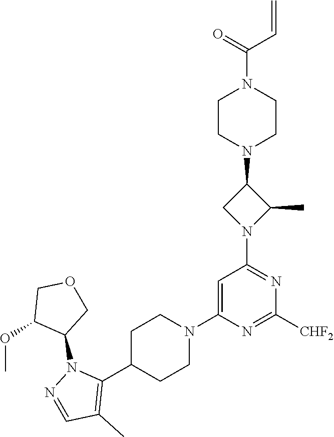 [SMILES: C=CC(=O)N1CCN([C@@H]2CN(c3cc(N4CCC(c5c(C)cnn5[C@@H]5COC[C@H]5OC)CC4)nc(C(F)F)n3)[C@@H]2C)CC1] | 1-(4-((2R,3R)-1-(2- (difluoromethyl)-6- (4-(1-((3R,4S)-4- methoxytetrabydro- 3-furanyl)-4- methyl-1H- pyrazol-5-yl)-1- piperidinyl)-4- pyrimidinyl)-2- methyl-3- azetidinyl)-1- piperazinyl)-2- propen-1-one | 2-597 | 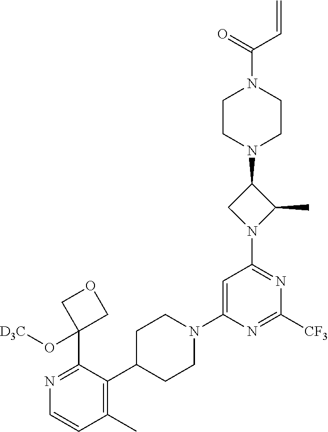 [SMILES: [2H]C([2H])([2H])OC1(c2nccc(C)c2C2CCN(c3cc(N4C[C@@H](N5CCN(C(=O)C=C)CC5)[C@H]4C)nc(C(F)(F)F)n3)CC2)COC1] | 1-(4-((2R,3R)-1-(6- (4-(2-(3-(methoxy- d3)oxetan-3-y])-4- methylpyridin-3- yl)piperidin-1-yl)- 2-(trifluoromethyl) pyrimidin-4-yl)-2- methylazetidin-3- yl)piperazin-1- yl)prop-2-en-1-one |
| 2-594 | 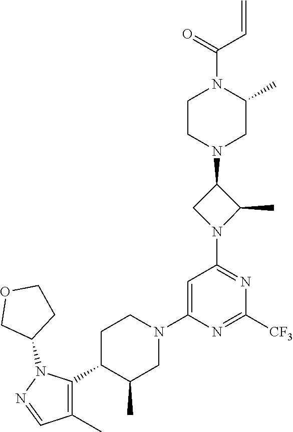 [SMILES: C=CC(=O)N1CCN([C@@H]2CN(c3cc(N4CC[C@@H](c5c(C)cnn5[C@H]5CCOC5)[C@H](C)C4)nc(C(F)(F)F)n3)[C@@H]2C)C[C@H]1C] | 1-((2R)-2-methyl- 4-((2R,3R)-2- methyl-1-(6- ((3S,4R)-3-methyl- 4-(4-methyl-1- ((3S)-tetrahydro-3- furanyl)-1H- pyrazol-5-yl)-1- piperidinyl)-2- (trifluoromethy])- 4-pyrimidinyl)-3- azetidinyl)-1- piperazinyl)-2- propen-1-one | 2-598 | 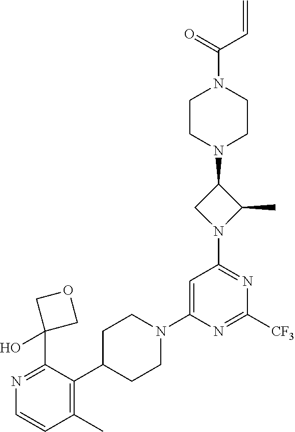 [SMILES: C=CC(=O)N1CCN([C@@H]2CN(c3cc(N4CCC(c5c(C)ccnc5C5(O)COC5)CC4)nc(C(F)(F)F)n3)[C@@H]2C)CC1] | 1-(4-((2R,3R)-1-(6- (4-(2-(3-hydroxy- 3-oxetanyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-595 | 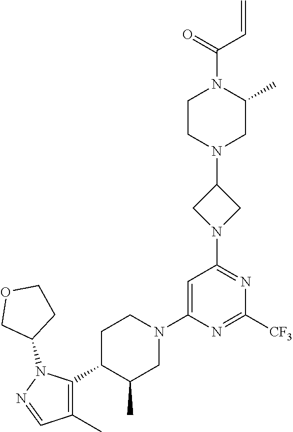 [SMILES: C=CC(=O)N1CCN(C2CN(c3cc(N4CC[C@@H](c5c(C)cnn5[C@H]5CCOC5)[C@H](C)C4)nc(C(F)(F)F)n3)C2)C[C@H]1C] | 1-((2R)-2-methyl- 4-(1-(6-((38,4R)-3- methyl-4-(4- methyl-1-((3S)- tetrahydro-3- furanyl)-1H- pyrazol-5-yl)-1- piperidinyl)-2- (trifluoromethyl)- 4-pyrintidinyl)-3- azetidinyl)-1- piperazinyl)-2- propen-1-one | 2-599 | 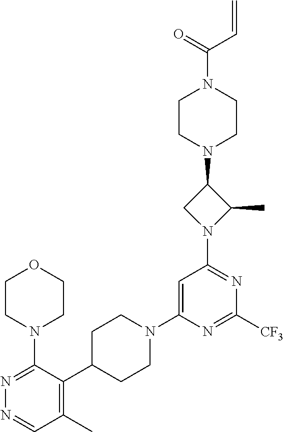 [SMILES: C=CC(=O)N1CCN([C@@H]2CN(c3cc(N4CCC(c5c(C)cnnc5N5CCOCC5)CC4)nc(C(F)(F)F)n3)[C@@H]2C)CC1] | 1-(4-((2R,3R)-2- methyl-1-(6-(4-(5- methyl-3-(4- morpholinyl)-4- pyridaziny])-1- piperidinyl)-2- (trifluoromethyl)- 4-pyrimidinyl)-3- azetidinyl)-1- piperazinyl)-2- propen-t-one |
| 2-600 | 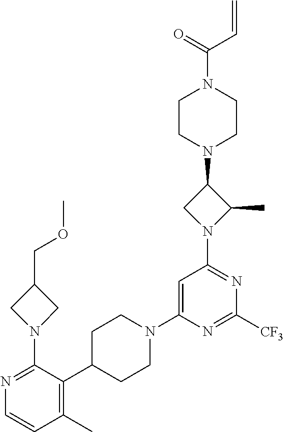 [SMILES: C=CC(=O)N1CCN([C@@H]2CN(c3cc(N4CCC(c5c(C)ccnc5N5CC(COC)C5)CC4)nc(C(F)(F)F)n3)[C@@H]2C)CC1] | 1-(4-((2R,3R)-1-(6- (4-(2-(3- (methoxymethyl)- l-azetidinyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one | 2-604 | 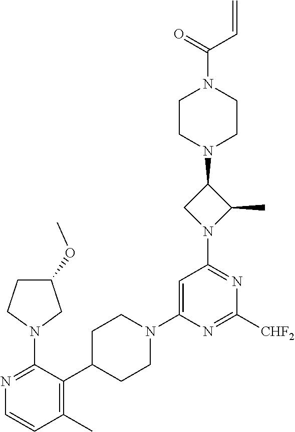 [SMILES: C=CC(=O)N1CCN([C@@H]2CN(c3cc(N4CCC(c5c(C)ccnc5N5CC[C@H](OC)C5)CC4)nc(C(F)F)n3)[C@@H]2C)CC1] | 1-(4-((2R,3R)-1-(2- (difluoromethyl)-6- (4-(2-((3S)-3- methoxy-1- pyrrolidinyl)-4- methyl-3- pyridinyl)-1- pipcridinyl)-4- pyrimidinyl)-2- methyl-3- azetidinyl)-1- piperazinyl)-2- propen-1-one |
| 2-601 | 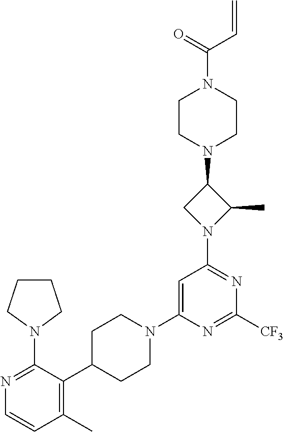 [SMILES: C=CC(=O)N1CCN([C@@H]2CN(c3cc(N4CCC(c5c(C)ccnc5N5CCCC5)CC4)nc(C(F)(F)F)n3)[C@@H]2C)CC1] | 1-(4-((2R,3R)-2- methyl-1-(6-(4-(4- methyl-2-(1- pyrrolidinyl)-3- pyridinyl)-1- piperidinyl)-2- (trifluoromethyl)- 4-pyrimidinyl)-3- azetidinyl)-1- piperazinyl)-2- propen-1-one | 2-605 | 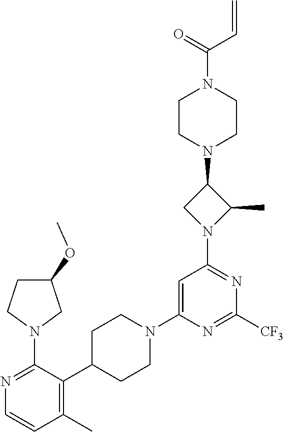 [SMILES: C=CC(=O)N1CCN([C@@H]2CN(c3cc(N4CCC(c5c(C)ccnc5N5CC[C@@H](OC)C5)CC4)nc(C(F)(F)F)n3)[C@@H]2C)CC1] | 1-(4-((2R,3R)-1-(6- (4-(2-((3R)-3- methoxy-1- pyrrolidinyl)-4- metbyl-3- pyridinyl)-1- piperidinyl)-2- (trifluoromethyl)- 4-pyrintidiny])-2- methyl-3- azetidinyl)-1- piperazinyl)-2- propen-1-one |
| 2-602 | 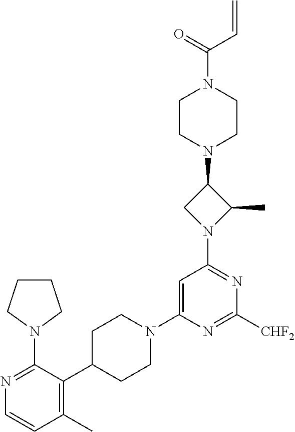 [SMILES: C=CC(=O)N1CCN([C@@H]2CN(c3cc(N4CCC(c5c(C)ccnc5N5CCCC5)CC4)nc(C(F)F)n3)[C@@H]2C)CC1] | 1-(4-((2R,3R)-1-(2- (difluoromethyl)-6- (4-(4-methyl-2-(1- pyrrolidinyl)-3- pyridinyl)-1- piperidiny])-4- pyrimidinyl)-2- methyl-3- azetidinyl)-1- piperazinyl)-2- propen-1-one | 2-606 | 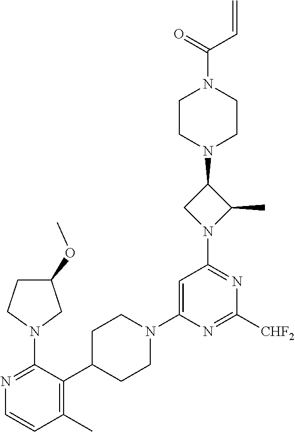 [SMILES: C=CC(=O)N1CCN([C@@H]2CN(c3cc(N4CCC(c5c(C)ccnc5N5CC[C@@H](OC)C5)CC4)nc(C(F)F)n3)[C@@H]2C)CC1] | 1-(4-((2R,3R)-1-(2- (difluoromethyl)-6- (4-(2-((3R)-3- methoxy-1* pyrrolidinyl)-4- methyl-3- pyridinyl)-1- piperidinyl)-4- pyrimidinyl)-2- methyl-3- azetidinyl)-1- piperazinyl)-2- propen-1-one |
| 2-603 | 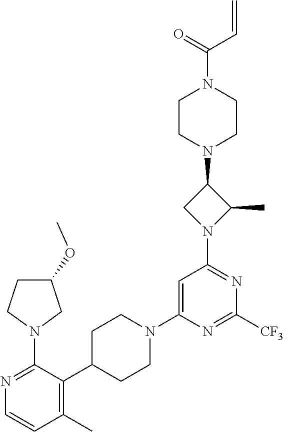 [SMILES: C=CC(=O)N1CCN([C@@H]2CN(c3cc(N4CCC(c5c(C)ccnc5N5CC[C@H](OC)C5)CC4)nc(C(F)(F)F)n3)[C@@H]2C)CC1] | 1-(4-((2R,3R)-1-(6- (4-(2-((3S)-3- methoxy-1- pyrrolidinyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one | 2-607 | 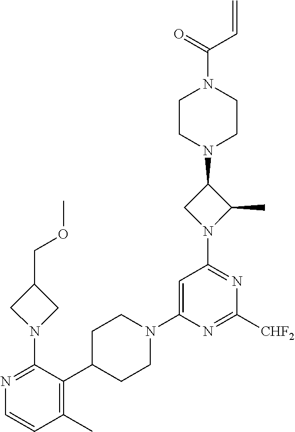 [SMILES: C=CC(=O)N1CCN([C@@H]2CN(c3cc(N4CCC(c5c(C)ccnc5N5CC(COC)C5)CC4)nc(C(F)F)n3)[C@@H]2C)CC1] | 1-(4-((2R,3R)-1-(2- (difluoromethyl)-6- (4-(2-(3- (methoxymethyl)- l-azetidinyl)-4- methyl-3- pyridinyl)-1- piperidinyl)-4- pyrimidinyl)-2- methyl-3- azetidinyl)-1- piperazinyl)-2- propen-1-one |
| 2-608 | 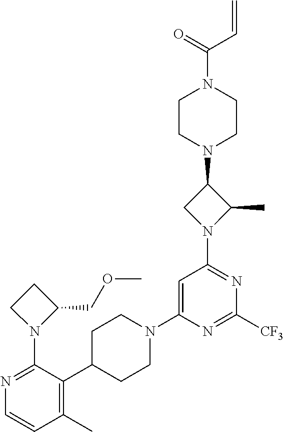 [SMILES: C=CC(=O)N1CCN([C@@H]2CN(c3cc(N4CCC(c5c(C)ccnc5N5CC[C@@H]5COC)CC4)nc(C(F)(F)F)n3)[C@@H]2C)CC1] | 1-(4-((2R,3R)-1-(6- (4-(2-((2R)-2- (methoxymethyl)- 1-azetidinyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one | 2-612 | 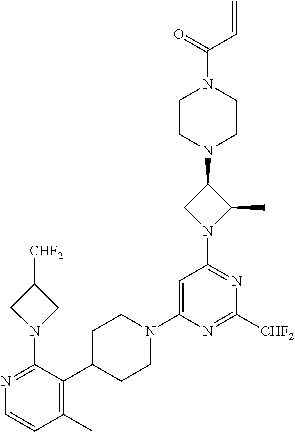 [SMILES: C=CC(=O)N1CCN([C@@H]2CN(c3cc(N4CCC(c5c(C)ccnc5N5CC(C(F)F)C5)CC4)nc(C(F)F)n3)[C@@H]2C)CC1] | 1-(4-((2R,3R)-1-(2- (difluoromethyl)-6- (4-(2-(3- (difluoromethyl)-1- azetidinyl)-4- methyl-3- pyridinyl)-1- pipcridinyl)-4- pyrimidinyl)-2- methyl-3- azetidinyl)-1- piperazinyl)-2- propen-1-one |
| 2-609 | 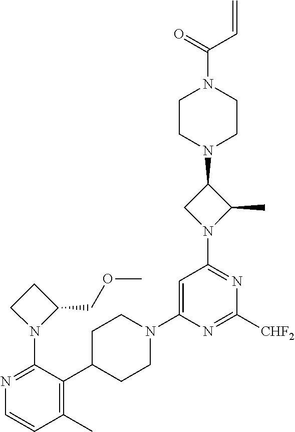 [SMILES: C=CC(=O)N1CCN([C@@H]2CN(c3cc(N4CCC(c5c(C)ccnc5N5CC[C@@H]5COC)CC4)nc(C(F)F)n3)[C@@H]2C)CC1] | 1-(4-((2R,3R)-1-(2- (difluoromethyl)-6- (4-(2-((2R)-2- (methoxymethyl)- 1-azetidinyl)-4- methyl-3- pyridinyl)-1- piperidinyl)-4- pyrimidinyl)-2- methyl-3- azetidinyl)-1- piperazinyl)-2- propen-1-one | 2-613 | 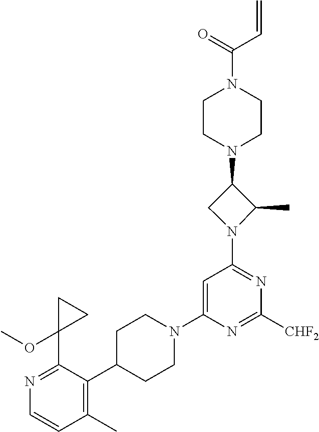 [SMILES: C=CC(=O)N1CCN([C@@H]2CN(c3cc(N4CCC(c5c(C)ccnc5C5(OC)CC5)CC4)nc(C(F)F)n3)[C@@H]2C)CC1] | 1-(4-((2R,3R)-1-(2- (difluoromcthyl)-6- (4-(2-(1- methoxycyclo- propyl)-4- methyl-3- pyridinyl)-1- piperidinyl)-4- pyrimidinyl)-2- methyl-3- azetidinyl)-1- piperazinyl)-2- propen-1-one |
| 2-610 | 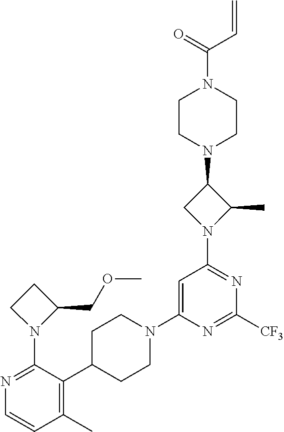 [SMILES: C=CC(=O)N1CCN([C@@H]2CN(c3cc(N4CCC(c5c(C)ccnc5N5CC[C@H]5COC)CC4)nc(C(F)(F)F)n3)[C@@H]2C)CC1] | 1-(4-((2R,3R)-1-(6- (4-(2-((2S)-2- (methoxymethyl)- l-azetidinyl)-4- methyl-3- pyridinyl)-1- piperidiny])-2- (trifluoromethyl)- 4-pyrimidinyl)-2- methyl-3- azetidinyl-1- piperazinyl)-2- propen-1-one | 2-614 | 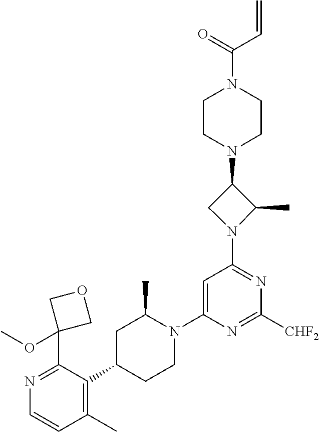 [SMILES: C=CC(=O)N1CCN([C@@H]2CN(c3cc(N4CC[C@H](c5c(C)ccnc5C5(OC)COC5)C[C@H]4C)nc(C(F)F)n3)[C@@H]2C)CC1] | 1-(4-((2R,3R)-1-(2- (difluoromethyl)-6- ((2R,4S)-4-(2-(3- methoxy-3- oxetanyl)-4- methyl-3- pyridinyl)-2- methyl-1- piperidinyl)-4- pyrimidinyl)-2- methyl-3- azetidinyl)-1- piperazinyl)-2- propen-1-one |
| 2-611 | 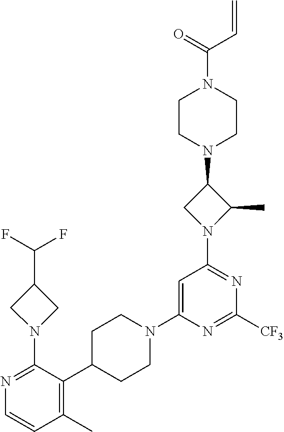 [SMILES: C=CC(=O)N1CCN([C@@H]2CN(c3cc(N4CCC(c5c(C)ccnc5N5CC(C(F)F)C5)CC4)nc(C(F)(F)F)n3)[C@@H]2C)CC1] | 1-(4-((2R,3R)-1-(6- (4-(2-(3- (difluoromethyl)-1- azetidinyl)-4- methyl-3- pyridinyl)-1- piperidinyl)-2- (trifluoromethyl)- 4-pyrimidinyl)-2- methyl-3- azetidinyl)-1- piperaziny])-2- propen-1-one | 2-615 | 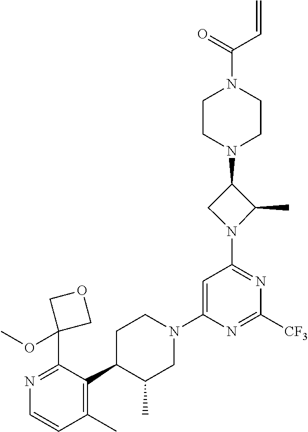 [SMILES: C=CC(=O)N1CCN([C@@H]2CN(c3cc(N4CC[C@H](c5c(C)ccnc5C5(OC)COC5)[C@@H](C)C4)nc(C(F)(F)F)n3)[C@@H]2C)CC1] | 1-(4-((2R,3R)-1-(6- ((3R,4S)-4-(2-(3- methoxy-3- oxetanyl)-4- methyl-3- pyridinyl)-3- methyl-1- piperidinyl)-2- (trifluoromethyl)- 4-pyrimidinyl)-2- methyl-3- azetidinyl)-1- piperazinyl)-2- propen-1-one |
| | 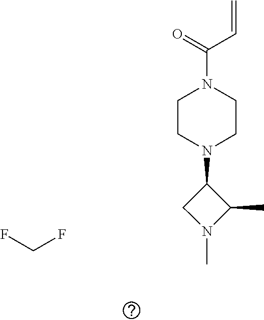 | | | | |
| 2-616 | 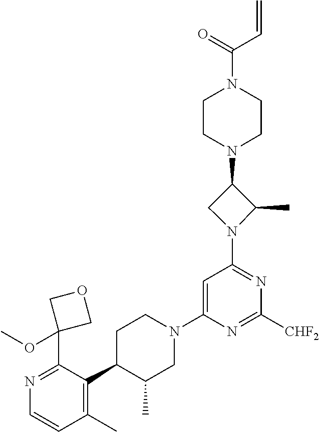 [SMILES: C=CC(=O)N1CCN([C@@H]2CN(c3cc(N4CC[C@H](c5c(C)ccnc5C5(OC)COC5)[C@@H](C)C4)nc(C(F)F)n3)[C@@H]2C)CC1] | 1-(4-((2R,3R)-1-(2- (difluorometlyl)-6- ((3R,4S)-4-(2-(3- methoxy-3- oxetanyl)-4- methyl-3- methyl-1- piperidiny])-4- pyrimidinyl)-2- methyl-3- azetidinyl)-1- piperazinyl)-2- propen-1-one | 2-620 | 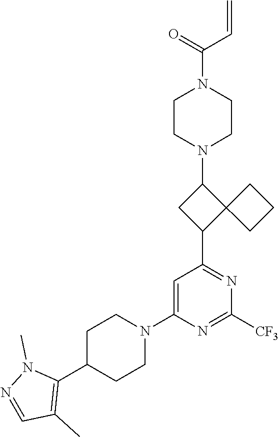 [SMILES: C=CC(=O)N1CCN(C2CC(c3cc(N4CCC(c5c(C)cnn5C)CC4)nc(C(F)(F)F)n3)C23CCC3)CC1] | 1-(4-(3-(6-(4-(1,4- dimethyl-1H- pyrazol-5-yl)-1- piperidinyl)-2- (trifluoromethyl)- 4- pyrimidinyl)spiro [3.3]heptan-1-yl)-1- piperazinyl)-2- propen-1-one |
| | | | | mixture of cis/trans isomers | |
| 2-617 | 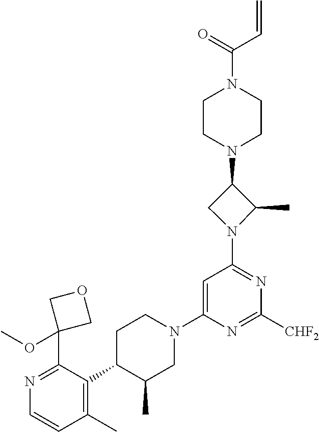 [SMILES: C=CC(=O)N1CCN([C@@H]2CN(c3cc(N4CC[C@@H](c5c(C)ccnc5C5(OC)COC5)[C@H](C)C4)nc(C(F)F)n3)[C@@H]2C)CC1] | 1-(4-((2R.3R)-1-(2- (difluoromethyl)-6- ((3S,4R)-4-(2-(3- methoxy-3- oxetanyl)-4- methyl-3- pytidinyl)-3- methyl-1- piperidinyl)-4- pyrimidinyl)-2- methyl-3- azetidinyl)-1- piperazinyl)-2- propen-1-one | 2-621 | 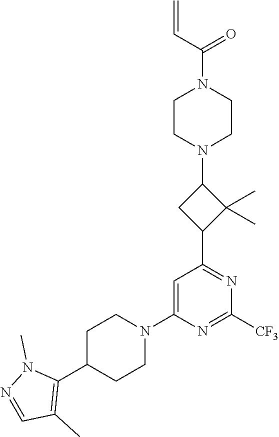 [SMILES: C=CC(=O)N1CCN(C2CC(c3cc(N4CCC(c5c(C)cnn5C)CC4)nc(C(F)(F)F)n3)C2(C)C)CC1] | 1-(4-(3-(6-(4-(1,4- dimethyl-1H- pyrazol-5-yl)-1- piperidinyl)-2- (trifluoromethyl)- 4-pyrimidinyl)-2,2- dimethylcyclo- butyl)-1- piperazinyl)-2- propen-t-one |
| | | | | mixture of cis/trans isomers | |
| 2-618 | 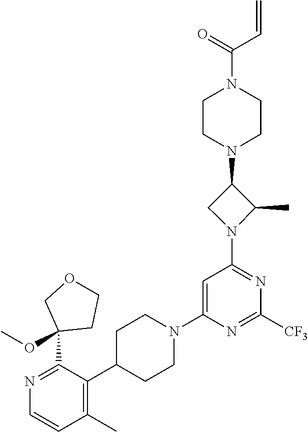 [SMILES: C=CC(=O)N1CCN([C@@H]2CN(c3cc(N4CCC(c5c(C)ccnc5[C@@]5(OC)CCOC5)CC4)nc(C(F)(F)F)n3)[C@@H]2C)CC1] | 1-(4-((2R,3R)-1-(6- (4-(2-((35)-3- methoxytetra- hydro-3- furanyl)-4- methyl-3- pyridinyl)-1- piperidiny])-2- (trifluoromethyl)- 4-pyrimidinyl)-2- methyl-3- azetidinyl)-1- piperazinyl)-2- propen-1-one | 2-622 | 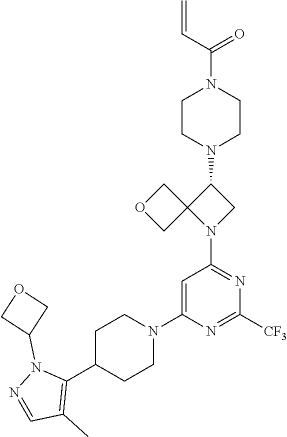 [SMILES: C=CC(=O)N1CCN([C@@H]2CN(c3cc(N4CCC(c5c(C)cnn5C5COC5)CC4)nc(C(F)(F)F)n3)C23COC3)CC1] | 1-(4-((3R)-1-(6-(4- (4-Methyl-1-(3- oxetanyl)-1H- pyrazol-5-yl)-1- piperidinyl)-2- (trifluoromethyl)- 4-pyrimidinyl)-6- oxa-1- azaspiro[3.3] heptan-3-yl)-1- piperazinyl)-2- propen-1-one |
| | racemic mixture | | | | |
| 2-619 | 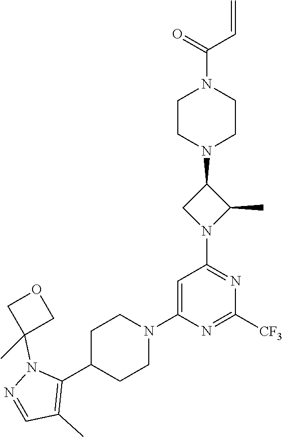 [SMILES: C=CC(=O)N1CCN([C@@H]2CN(c3cc(N4CCC(c5c(C)cnn5C5(C)COC5)CC4)nc(C(F)(F)F)n3)[C@@H]2C)CC1] | 1-(4-((2R,3R)-2- methyl-1-(6-(4-(4- methyl-1-(3- methyl-3- oxetanyl)-1H- pyrazol-5-yl)-1- piperidiny])-2- (trifluoromethyl)- 4-pyrimidinyl)-3- azetidiny])-1- piperazinyl)-2- propen-1-one | 2-623 | 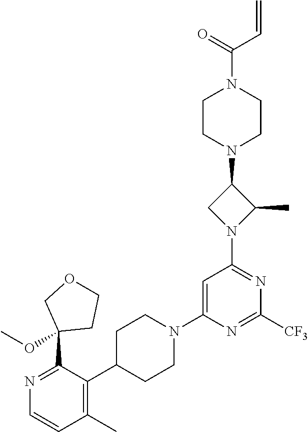 [SMILES: C=CC(=O)N1CCN([C@@H]2CN(c3cc(N4CCC(c5c(C)ccnc5[C@]5(OC)CCOC5)CC4)nc(C(F)(F)F)n3)[C@@H]2C)CC1] | 1-(4-((2R,3R)-1-(6- (4-(2-((3R)-3- methoxytetra- hydro-3-furanyl)- 4-methyl-3- pyridinyl)-1- piperidinyl)-2- (trifluoromethyl)- 4-pyrimidinyl)-2- methyl-3- azetidinyl)-1- piperazinyl)-2- propen-1-one |
| 2-624 | 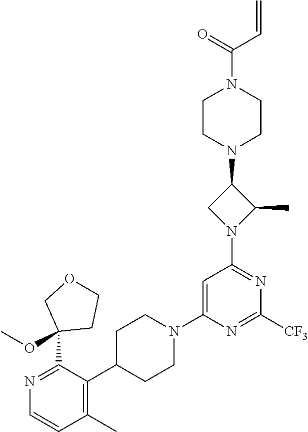 [SMILES: C=CC(=O)N1CCN([C@@H]2CN(c3cc(N4CCC(c5c(C)ccnc5[C@@]5(OC)CCOC5)CC4)nc(C(F)(F)F)n3)[C@@H]2C)CC1] | 1-(4-((2R,3R)-1-(6- (4-(2-((3S)-3- methoxytetrabydro- 3-furanyl)-4- methyl-3- piperidinyl)-2- (trifluoromethyl)- 4-pyrimidinyl)-2- methyl-3- azetidinyl)-1- piperazinyl)-2- propen-1-one | 2-628 | 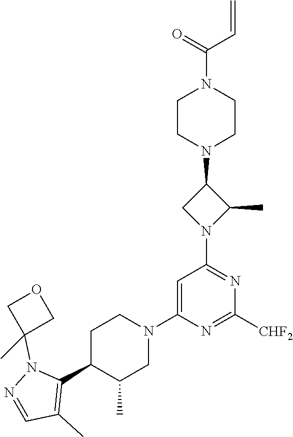 [SMILES: C=CC(=O)N1CCN([C@@H]2CN(c3cc(N4CC[C@H](c5c(C)cnn5C5(C)COC5)[C@@H](C)C4)nc(C(F)F)n3)[C@@H]2C)CC1] | 1-(4-((2R,3R)-1-(2- (difluoromethyl)-6- ((3R,4S)-3-methyl- 4-(4-methyl-1-(3- methyl-3- oxetany])-1H- pyrazol-5-yl)-1- pipcridinyl)-4- pyrimidinyl)-2- methyl-3- azetidinyl)-1- piperazinyl)-2- propen-1-one |
| 2-625 | 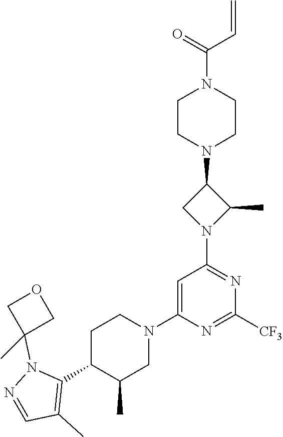 [SMILES: C=CC(=O)N1CCN([C@@H]2CN(c3cc(N4CC[C@@H](c5c(C)cnn5C5(C)COC5)[C@H](C)C4)nc(C(F)(F)F)n3)[C@@H]2C)CC1] | 1-(4-((2R,3R)-2- methyl-1-(6- ((3S,4R)-3-methyl- 4-(4-methyl-1-(3- methyl-3- oxetanyl)-1H- pyrazol-5-yl)-1- piperidinyl)-2- (trifluoromethyl)- 4-pyrimidinyl)-3- azetidinyl)-1- piperazinyl)-2- propen-1-one | 2-629 | 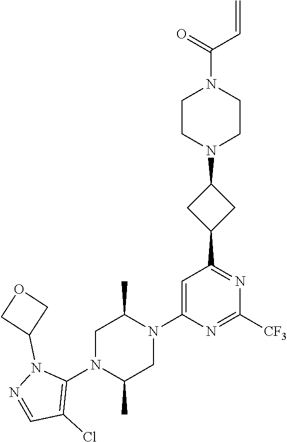 [SMILES: C=CC(=O)N1CCN([C@H]2C[C@@H](c3cc(N4C[C@@H](C)N(c5c(Cl)cnn5C5COC5)C[C@H]4C)nc(C(F)(F)F)n3)C2)CC1] | 1-(4-(cis-3-(6- ((2R,5R)-4-(4- chloro-1-(3- oxetanyl)-1H- pyrazol-5-yl)-2,5- dimethyl-1- piperazinyl)-2- (trifluoromethyl)- 4-pyrimidinyl) cyclobutyl)-1- piperazinyl)-2- propen-1-one |
| 2-626 | 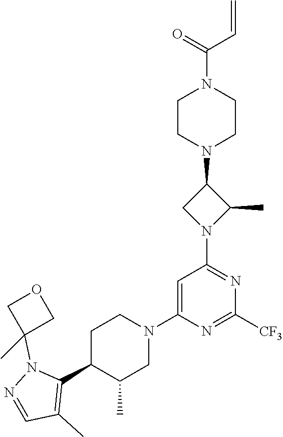 [SMILES: C=CC(=O)N1CCN([C@@H]2CN(c3cc(N4CC[C@H](c5c(C)cnn5C5(C)COC5)[C@@H](C)C4)nc(C(F)(F)F)n3)[C@@H]2C)CC1] | 1-(4-((2R,3R)-2- methyl-1-(6- ((3R,4S)-3-methyl- 4-(4-methy]-1-(3- methyl-3- oxetanyl)-1H- pyrazol-5-yl)-1- piperidinyl)-2- (trifluoromethyl)- 4-pyrimidinyl)-3- azetidinyl-[- piperazinyl)-2- propen-1-one | 2-630 | 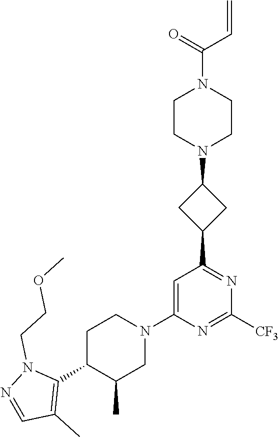 [SMILES: C=CC(=O)N1CCN([C@H]2C[C@@H](c3cc(N4CC[C@@H](c5c(C)cnn5CCOC)[C@H](C)C4)nc(C(F)(F)F)n3)C2)CC1] | 1-(4-(cis-3-(6- ((35,4R)-4-(1-(2- methoxyethyl)-4- methyl-1H- pyrazol-5-yl)-3- methyl-1- piperidinyl)-2- (trifluoromethyl)- 4-pyrimidinyl) cyclobutyl)-1- piperazinyl)-2- propen-1-one |
| 2-627 | 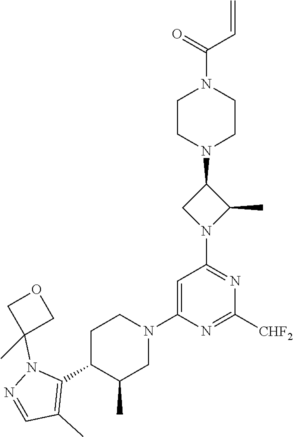 [SMILES: C=CC(=O)N1CCN([C@@H]2CN(c3cc(N4CC[C@@H](c5c(C)cnn5C5(C)COC5)[C@H](C)C4)nc(C(F)F)n3)[C@@H]2C)CC1] | 1-(4-((2R,3R)-1-(2- (difluoromethyl)-6- ((3S,4R)-3-methyl- 4-(4-methyl-1-(3- methyl-3- oxctabyl)-1H- pyrazol-5-yl)-1- piperidinyl)-4- pyrimidinyl)-2- methyl-3- azetidinyl)-1- piperazinyl)-2- propen-1-one | 2-631 | 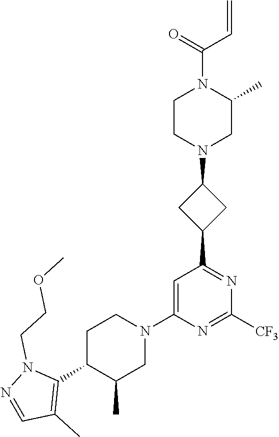 [SMILES: C=CC(=O)N1CCN([C@H]2C[C@@H](c3cc(N4CC[C@@H](c5c(C)cnn5CCOC)[C@H](C)C4)nc(C(F)(F)F)n3)C2)C[C@H]1C] | 1-((2R)-4-(cis-3- (6-((3S,4R)-4-(1- (2-methoxyethy])- 4-methyl-1H- pyrazol-5-yl)-3- methyl-1- piperidinyl)-2- (trifluoromethyl)- 4.pyrimidinyl) cyclobutyl)-2- methyl-1- piperaziny])-2- propen-1-one |
| 2-632 | 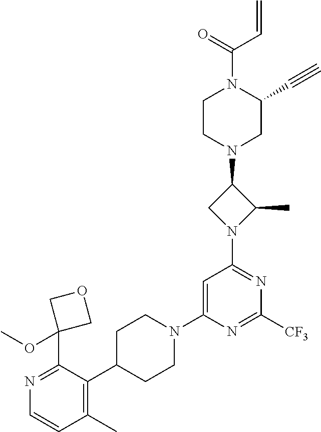 [SMILES: C#C[C@@H]1CN([C@@H]2CN(c3cc(N4CCC(c5c(C)ccnc5C5(OC)COC5)CC4)nc(C(F)(F)F)n3)[C@@H]2C)CCN1C(=O)C=C] | 1-((2R)-2-ethynyl- 4-((2R.3R)-1-(6-(4- (2-(3-methoxy-3- oxetanyl)-4- methyl-3- pyridinyl)-1- piperidinyl)-2- (trifluoromethyl)- 4-pyrimidinyl)-2- methyl-3- azetidinyl)-1- piperazinyl)-2- propen-1-one | 2-636 | 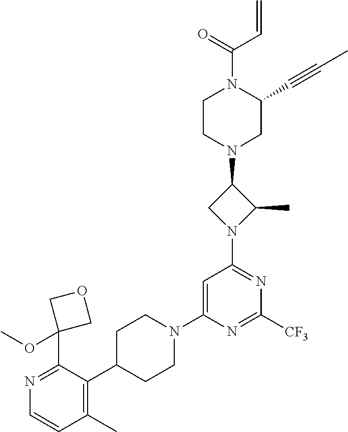 [SMILES: C=CC(=O)N1CCN([C@@H]2CN(c3cc(N4CCC(c5c(C)ccnc5C5(OC)COC5)CC4)nc(C(F)(F)F)n3)[C@@H]2C)C[C@H]1C#CC] | 1-((2R)-4- ((2R,3R)-1-(6-(4- (2-(3-methoxy-3- oxetanyl)-4- methyl-3- pyridinyl)-1- piperidinyl)-2- (trifluoromethyl)- 4-pyrimidinyl)-2- methyl-3- azetidinyl)-2-(1- propyn-1-yl)-1- piperazinyl)-2- propen-1-one |
| 2-633 | 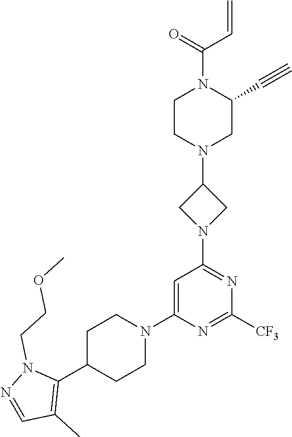 [SMILES: C#C[C@@H]1CN(C2CN(c3cc(N4CCC(c5c(C)cnn5CCOC)CC4)nc(C(F)(F)F)n3)C2)CCN1C(=O)C=C] | 1-((2R)-2-ethynyl- 4-(1-(6-(4-(1-(2- methoxyethyl)-4- methyl-1H- pyrazol-5-yl)-1- piperidinyl)-2- (trifluoromethyl)- 4-pyrimidinyl)-3- azetidinyl)-1- piperazinyl)-2- propen-1-one | 2-637 | 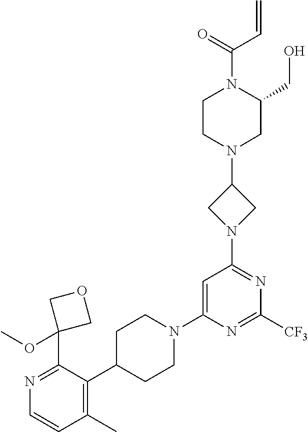 [SMILES: C=CC(=O)N1CCN(C2CN(c3cc(N4CCC(c5c(C)ccnc5C5(OC)COC5)CC4)nc(C(F)(F)F)n3)C2)C[C@H]1CO] | 1-((2S)-2- (hydroxymethyl)- 4-(1-(6-(4-(2-(3- methoxy-3- oxelanyl)-4- methyl-3- pyridinyl)-1- piperidinyl)-2- (trifluoromethyl)- 4-pyrimidinyl)-3- azetidinyl)-1- piperazinyl)-2- propen-1-one |
| 2-634 | 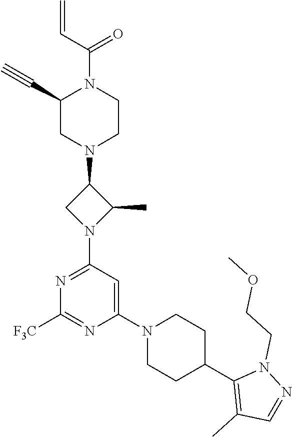 [SMILES: C#C[C@@H]1CN([C@@H]2CN(c3cc(N4CCC(c5c(C)cnn5CCOC)CC4)nc(C(F)(F)F)n3)[C@@H]2C)CCN1C(=O)C=C] | 1-((2R)-2-cthynyl- 4-((2R,3R)-1-(6-(4- (1-(2- methoxyethyl)-4- methyl-1H- pyrazol-5-yl)-1- piperidiny])-2- (trifluoromethyl)- 4-pyrimidinyl)-2- methyl-3- azetidinyl)-1- piperazinyl)-2- propen-1-one | 2-638 | 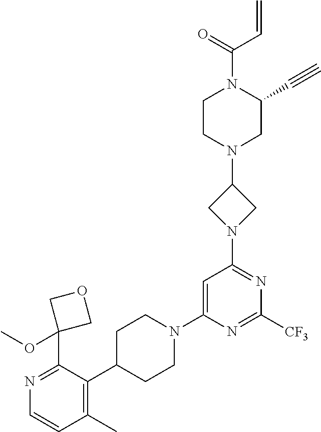 [SMILES: C#C[C@@H]1CN(C2CN(c3cc(N4CCC(c5c(C)ccnc5C5(OC)COC5)CC4)nc(C(F)(F)F)n3)C2)CCN1C(=O)C=C] | 1-((2R)-2-ethynyl- 4-(1-(6-(4-(2-(3- methoxy-3- oxetanyl)-4- methyl-3- pyridinyl)-1- piperidinyl)-2- (trifluoromethyl)- 4-pyrimidinyl)-3- azetidinyl)-1- piperazinyl)-2- propen-1-one |
| 2-635 (mix) | 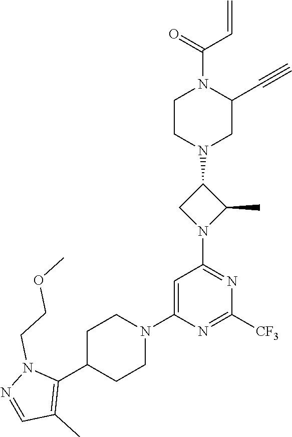 [SMILES: C#CC1CN([C@H]2CN(c3cc(N4CCC(c5c(C)cnn5CCOC)CC4)nc(C(F)(F)F)n3)[C@@H]2C)CCN1C(=O)C=C] | 1-(2-ethynyl-4- ((2R,3S)-1-(6-(4- (1-(2- methoxyethyl)-4- methyl-1H- pyrazol-5-yl)-1- piperidinyl)-2- (trifluoromethyl)- 4-pyrimidinyl)-2- methyl-3- azetidinyl)-1- piperazinyl)-2- propen-1-one | 2-639 | 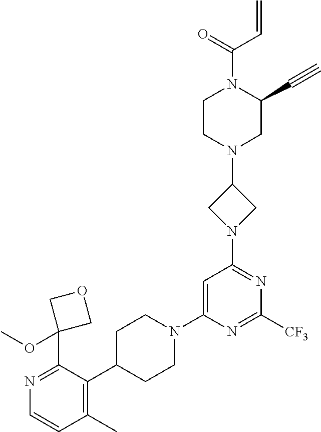 [SMILES: C#C[C@H]1CN(C2CN(c3cc(N4CCC(c5c(C)ccnc5C5(OC)COC5)CC4)nc(C(F)(F)F)n3)C2)CCN1C(=O)C=C] | 1-((2S)-2-ethynyl- 4-(1-(6-(4-(2-(3- methoxy-3- oxetanyl)-4- methyl-3- pyridinyl)-1- piperidinyl)-2- (trifluoromethyl)- 4-pyrimidinyl)-3- azetidinyl)-1- piperazinyl)-2- propen-1-one |
| | racemic mixture | | | | |
| 2-640 | 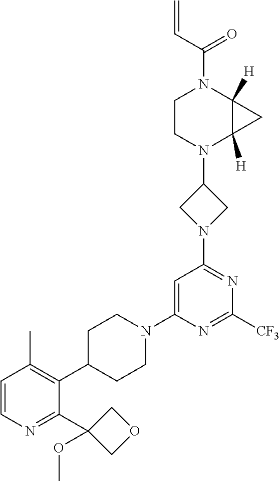 [SMILES: C=CC(=O)N1CCN(C2CN(c3cc(N4CCC(c5c(C)ccnc5C5(OC)COC5)CC4)nc(C(F)(F)F)n3)C2)[C@H]2C[C@H]21] | 1-((1R,6S)-5-(1-(6- (4-(2-(3-methoxy- 3-oxetanyl)-4- methyl-3- pyridinyl)-1- piperidinyl)-2- (trifluoromethyl)- 4-pyrimidiny])-3- azetidinyl)-2,5- diazabicyclo[4.1.0] heptan-2-yl)-2- propen-1-one | 2-645 | 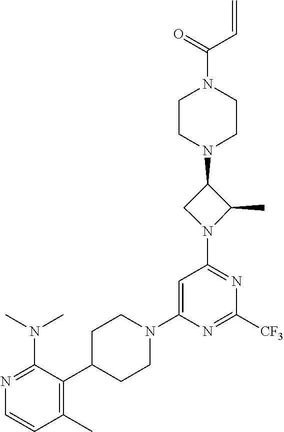 [SMILES: C=CC(=O)N1CCN([C@@H]2CN(c3cc(N4CCC(c5c(C)ccnc5N(C)C)CC4)nc(C(F)(F)F)n3)[C@@H]2C)CC1] | 1-(4-((2R.3R)-1-(6- (4-(2- (dimethylamino)-4- methylpyridin-3- yl)piperidin-1-yl)- 2-(trifluoromethyl) pyrimidin-4-yl)-2- methylazetidin-3- yl)piperazin-1- yl)prop-2-en-1-one |
| 2-641 | 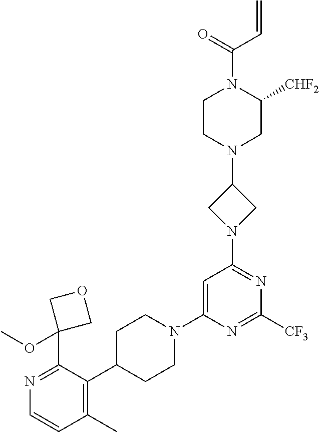 [SMILES: C=CC(=O)N1CCN(C2CN(c3cc(N4CCC(c5c(C)ccnc5C5(OC)COC5)CC4)nc(C(F)(F)F)n3)C2)C[C@H]1C(F)F] | 1-((28)-2- (difluoromethyl)-4- (1-(6-(4-(2-(3- methoxy-3- oxetanyl)-4- methyl-3- pyridinyl)-1- piperidinyl)-2- (trifluoromethy])- 4-pyrimidinyl)-3- azetidinyl)-1- piperazinyl)-2- propen-1-one | 2-646 | 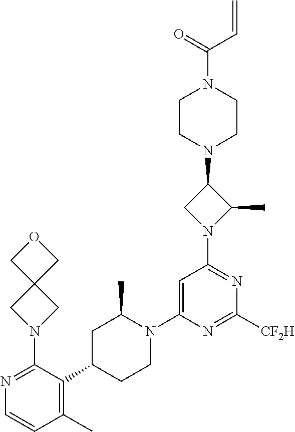 [SMILES: C=CC(=O)N1CCN([C@@H]2CN(c3cc(N4CC[C@H](c5c(C)ccnc5N5CC6(COC6)C5)C[C@H]4C)nc(C(F)F)n3)[C@@H]2C)CC1] | 1-(4-((2R,3R)-1-(2- (difluoromethyl)-6- ((2R,4S)-2-methyl- 4-(4-methyl-2-(2- oxa-6- azaspiro[3.3] heptan-6- yl)pyridin-3- yl)piperidin-1- yl)pyrimidin-4-yl)- 2-methylazetidin- 3-yl)piperazin-1- yl)prop-2-en-1-one |
| 2-642 | 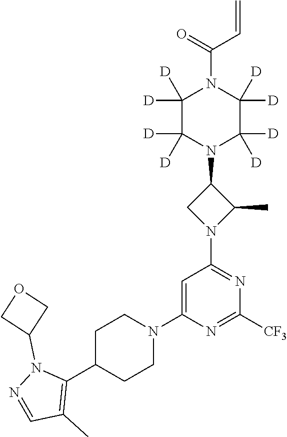 [SMILES: [2H]C1([2H])N(C(=O)C=C)C([2H])([2H])C([2H])([2H])N([C@@H]2CN(c3cc(N4CCC(c5c(C)cnn5C5COC5)CC4)nc(C(F)(F)F)n3)[C@@H]2C)C1([2H])[2H]] | 1-(4-((2R.3R)-2- methyl-1-(6-(4-(4- methyl-1-(oxetan- 3-yl)-1H-pyrazol- 5-yl)piperidin-1- yl)-2- (trifluoromethyl) pyrimidin-4- yl)azetidin-3- yl)piperazin-1-yl- 2,2,3,3,5,5,6,6- d8)prop-2-en-1-one | 2-647 | 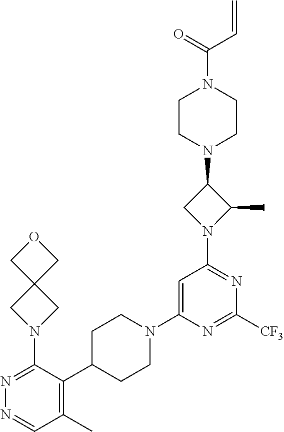 [SMILES: C=CC(=O)N1CCN([C@@H]2CN(c3cc(N4CCC(c5c(C)cnnc5N5CC6(COC6)C5)CC4)nc(C(F)(F)F)n3)[C@@H]2C)CC1] | 1-(4-((2R.3R)-2- methyl-1-16-(4-(5- methyl-3-(2-oxa-6- azaspiro[3.3] heptan-6- yl)pyridazin-4- yl)piperidin-1-yl)- 2-(trifluoromethyl) pyrimidin-4- yl)azetidin-3- yl)piperazin-1- yl)prop-2-en-1-one |
| 2-644 | 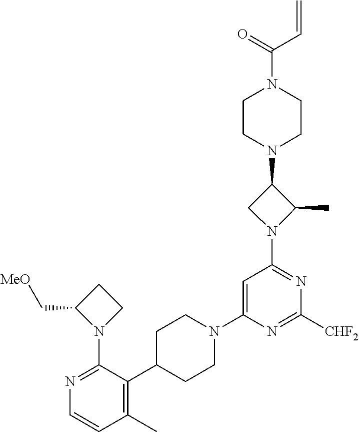 [SMILES: C=CC(=O)N1CCN([C@@H]2CN(c3cc(N4CCC(c5c(C)ccnc5N5CC[C@H]5COC)CC4)nc(C(F)F)n3)[C@@H]2C)CC1] | 1-(4-((2R,3R)-1-(2- (difluoromethyl)-6- (4-(2-((S)-2- (methoxymethyl) azctidin-1-yl)-4- methylpyridin-3- yl)piperidin-1- yl)pyrimidin-4-y])- 2-methylazetidin- 3-yl)piperazin-1- yl)prop-2-en-1-one | | | |

[symbol]

indicates data missing or illegible when filed

In some cases, the compound of Formula (I) selected from the list of compounds in Table E, or a pharmaceutically acceptable salt thereof. In some cases, the compound of Formula (I) is compound selected from compound 2-001 through compound 2-647.

In some cases, A is CH, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy; and X is

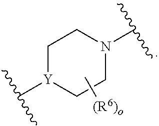

In some cases, the compound of Formula (I) and/or (IA) is selected from compound 2-137, 2-138, 2-306 to 2-309, 2-335, 2-337, 2-350, 2-357, 2-367, 2-392, 2-392-1, 2-429, 2457, 2457-1, 2-462, 2462-1, 2477, 2477-1, 2478, 2-478-1, 2479, 2479-1, 2480, 2-481, 2486, 2488, 2-514, 2-514-1, 2-526, 2-620, 2-621, 2-622, 2-629, 2-630, and 2-631, or a pharmaceutically acceptable salt any of the foregoing.

In some cases, A is N and X is

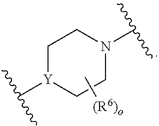

In some cases t compound of Formula (I) and/or Formula (IB) is selected from compound 2-001 to 2-136 and 2-139 to 2-305, 2-310 to 2-334, 2-336, 2-338 to 2-349, 2-351 to 2-355, 2-358 to 2-366, 2-368 to 2-370, 2-371 to 2-380, 2-382 to 2-391-1, 2-393 to 2-428-2, 2-430 to 2-453, 2-455, 2-456, 2-458 to 2-461, 2-463 to 2-476, 2-482 to 2-485, 2-487, 2-489 to 2-513, 2-515 to 2-525, 2-527 to 2-586, 2-588 to 2-619, 2-623 to 2-628, 2-632 to 2-642, and 2-644 to 2-647, or a pharmaceutically acceptable salt any of the foregoing.

In some cases, A is N; X is

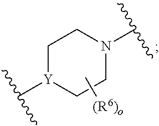

and Y is C—H, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy. In some cases, the compound of Formula (I) and/or Formula (IC) is selected from compound 2-001 to 2-007, 2-015, 2-022, 2-026 to 2-063, 2-067 to 2-133, 2-135 to 2-157, 2-159 to 2-165, 2-168 to 2-175, 2-177 to 2-180, 2-182 to 2-190, 2-193 to 2-204, 2-207 to 2-243, 2-245 to 2-282, 2-284 to 2-292, 2-294 to 2-305, 2-310, 2-312 to 2-334, 2-336, 2-339 to 2-349, 2-351 to 2-355, 2-358 to 2-366, 2-368 to 2-370, 2-371 to 2-380, 2-382 to 2-391-1, 2-393 to 2-428-2, 2-430 to 2-453, 2-455, 2-456, 2-458 to 2-461, 2-463 to 2-476, 2-482, 2-483, 2-485, 2-489 to 2-513, 2-515 to 2-525, 2-527 to 2-586, 2-588 to 2-619, 2-623 to 2-628, 2-632 to 2-642, and 2-644 to 2-647, or a pharmaceutically acceptable salt of any of the foregoing.

In some cases, A is N; X is

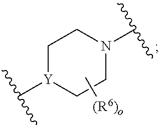

and Y is N. In some cases, and the compound of Formula (I) and/or Formula (ID) is selected from compound 2-008 to 2-014, 2-017 to 2-021, 2-023 to 2-025, 2-065, 2-066, 2-134, 2-158, 2-167, 2-176, 2-181, 2-244, 2-283, 2-293, 2-306, 2-484, and 2-487, or a pharmaceutically acceptable salt of any of the foregoing.

In some cases, A is N and X is

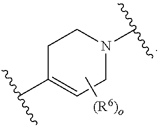

In some cases, the compound of Formula (I) and/or Formula (IE) is selected from compound 2-144, 2-191, 2-192, 2-205, 2-206, 2-311, and 2-454, or a pharmaceutically acceptable salt of any of the foregoing.

In some cases, A is N; X is

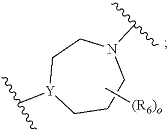

and Y is N. In some cases, the compound of Formula (I) and/or Formula (IF) is compound 2-338, or a pharmaceutically acceptable salt thereof.

In some cases, the disclosure provides compounds of Formula (T) wherein A is N; X is

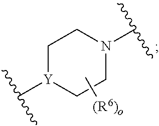

and Z is optionally substituted pyrazolyl. In some cases, the compound of Formula (I) is selected from compound: 2-001 to 2-014, 2-017 to 2-021, 2-023 to 2-025, 2-027, 2-029 to 2-031, 2-033, 2-035, 2-036, 2-039, 2-042, 2-044 to 2-048, 2-050 to 2-054, 2-057, 2-061 to 2-063, 2-067, 2-069 to 2-081, 2-084, to 2-088, 2-014 to 2-097, 2-099, 2-100, 2-103 to 2-130, 2-132, 2-133, 2-135 to 2-141, 2-143, 2-145 to 2-148, 2-151 to 2-155, 2-157, 2-159, 2-161, 2-162, 2-164, 2-168, 2-170, 2-171, 2-173, 2-176, 2-178 to 2-180, 2-182, 2-186 to 2-188, 2-190 to 2-194, 2-196, 2-198 to 2-202, 2-208 to 2-212, 2-216, 2-218, 2-219, 2-221 to 2-227, 2-229 to 2-232, 2-234 to 2-237, 2-242, 2-245 to 2-250, 2-253 to 2-256, 2-259, 2-261 to 2-269, 2-272 to 2-274, 2-276 to 2-278, 2-282 to 2-285, 2-290 to 2-295, 2-299, 2-303 to 2-313, 2-318 to 2-323, 2-329, 2-330, 2-333 to 2-342, 2-344, 2-345, 2-347 to 2-352, 2-354 to 2-357, 2-362, 2-363, 2-365 to 2-369, 2-371, 2-391, 2-391-1, 2-394 to 2-398, 2-400 to 2-404, 2-406, 2-408, 2-414, 2-418 to 2-422, 2-428, 2-428-1, 2-428-2, 2-430, 2-439, 2-439-1, 2-441, 2-442, 2-442, 2-446 to 2-449, 2-452, 2453, 2-456, 2-458, 2-460, 2-461, 2-465 to 2-471, 2-484, 2-485, 2-487, 2-489 to 2-492-1, 2-495 to 2-497, 2-499, 2-501 to 2-506, 2-508, 2-509, 2-511, 2-516, 2-516-1, 2-518, 2-520, 2-520-1, 2-520-2, 2-528, 2-536 to 2-551, 2-588 to 2-595, 2-619, 2-625 to 2-628, 2-633 to 2-635, and 2-642, or a pharmaceutically acceptable salt of any of the foregoing.

In some cases, the disclosure provides compounds of Formula (I) wherein A is N; X is

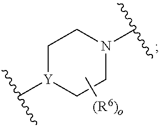

and Z is thiazolyl, isothiazolyl, oxazolyl, isoxazolyl, imidazolyl, or triazolyl. In some cases, the compound of Formula (I) is selected from compound: 2-026, 2-028, 2-032, 2-034, 2-037, 2-038, 2-40, 2-041, 2-043, 2-049, 2-055, 2-058, 2-059, 2-065 to 2-066, 2-068, 2-082, 2-083, 2-089 to 2-093, 2-101, 2-102, 2-131, 2-134, 2-142, 2-149, 2-150, 2-156, 2-158, 2-160, 2-163, 2-165, 2-167, 2-169, 2-172, 2-174, 2-175, 2-181, 2-184, 2-185, 2-195, 2-203, 2-204, 2-207, 2-213, 2-214, 2-217, 2-220, 2-228, 2-233, 2-239, 2-240, 2-244, 2-270, 2-271, 2-286 to 2-289, 2-296, 2-297, 2-298, 2-314, 2-315, 2-324, 2-331, 2-332, 2-343, 2-364, 2-370, 2-390, 2-407, 2-409, 2-409-1, 2-409-2, 2-411, 2-412, 2-415 to 2-417, 2-423 to 2-427, 2-507, 2-507-1, 2-507-2, 2-512, 2-532 to 2-534-1, and 2-552, or a pharmaceutically acceptable salt of an of the foregoing.

In some cases, the disclosure provides compounds of Formula (I) wherein A is N; X is

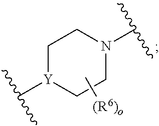

and Z is optionally substituted pyridyl. In some cases, the compound of Formula (I) is selected from compound: 2-056, 2-060, 2-098, 2-177, 2-189, 2-197, 2-205, 2-2-116, 2-215, 2-238, 2-241, 2-251, 2-252, 2-257, 2-258, 2-260, 2-275, 2-279 to 2-281, 2-300 to 2-302, 2-317, 2-325 to 2-327, 2-346, 2-353, 2-360, 2-361, 2-372 to 2-387-2, 2-384, 2-387, 2-389, 2-393, 2-399, 2-405, 2-410, 2-413, 2-431 to 2-433, 2-435 to 2-438, 2-440, 2-445, 2-450, 2-451, 2-455, 2-459, 2-464, 2-472 to 2-476, 2-493 to 2-494-1, 2-498, 2-500, 2-510, 2-510-1, 2-510-2, 2-513, 2-515, 2-517, 2-519, 2-521 to 2-525, 2-530 to 2-531-1, 2-535, 2-553 to 2-586, 2-596 to 2-598, 2-600 to 2-618, 2-623, 2-624, 2-632, 2-636 to 2-641, and 2-644 to 2-646, or a pharmaceutically acceptable salt of any of the foregoing.

In some cases, the disclosure provides compounds of Formula (I) wherein A is N; X is

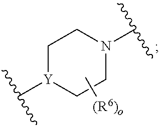

and Z is optionally substituted pyrazinyl. In some cases, the compound of Formula (I), is selected from compound: 2-379, 2-379, 2-383, 2-383, 2-385, 2-386, 2-388, 2-434, 2-443, 2-463, 2-527, 2-599, and 2-647, or a pharmaceutically acceptable salt of any of the foregoing.

In some cases, the disclosure provides compounds of Formula (I) wherein A is N; X is

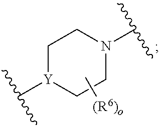

and Z is an optionally substituted bicyclic ring comprising a heteroaryl ring having 5 or 6 total ring atoms and 1-3 heteroatoms selected from N, O, and S fused to a ring having 5 or 6 total ring atoms and 0, 1, or 2 heteroatoms selected from N, O, and S. In some cases, the compound of Formula (I) is selected from compound: 2-015, 2-022, 2-243, 2-316, 2-328, 2-358, 2-359, 2, 482, and 2-483, or a pharmaceutically acceptable salt of any of the foregoing.

In some cases, the compound of Formula (I) is selected from the group consisting of 2-002, 2-003, 2-004, 2-067, 2-114, 2-115, 2-226, 2-227, 2-238, 2-249, 2-255, 2-258, 2-263, 2-299, 2-329, 2-336, 2-347, and 2-353, or a pharmaceutically acceptable salt of any of the foregoing. In some cases, the compound of Formula (I) is 2-002, or a pharmaceutically acceptable salt thereof. In some cases, the compound of Formula (I) is 2-003, or a pharmaceutically acceptable salt thereof. In some cases, the compound of Formula (I) is 2-004, or a pharmaceutically acceptable salt thereof. In some cases, the compound of Formula (I) is 2-067, or a pharmaceutically acceptable salt thereof. In some cases, the compound of Formula (I) is 2-114, or a pharmaceutically acceptable salt thereof. In some cases, the compound of Formula (I) is 2-115, or a pharmaceutically acceptable salt thereof. In some cases, the compound of Formula (I) is 2-226, or a pharmaceutically acceptable salt thereof. In some cases, the compound of Formula (I) is 2-227, or a pharmaceutically acceptable salt thereof. In some cases, the compound of Formula (I) is 2-238, or a pharmaceutically acceptable salt thereof. In some cases, the compound of Formula (I) is 2-249, or a pharmaceutically acceptable salt thereof. In some cases, the compound of Formula (I) is 2-255, or a pharmaceutically acceptable salt thereof. In some cases, the compound of Formula (I) is 2-258, or a pharmaceutically acceptable salt thereof. In some cases, the compound of Formula (I) is 2-263, or a pharmaceutically acceptable salt thereof. In some cases, the compound of Formula (I) is 2-299, or a pharmaceutically acceptable salt thereof. In some cases, the compound of Formula (I) is 2-329, or a pharmaceutically acceptable salt thereof. In some cases, the compound of Formula (I) is 2-336, or a pharmaceutically acceptable salt thereof. In some cases, the compound of Formula (I) is 2-347, or a pharmaceutically acceptable salt thereof. In some cases, the compound of Formula (I) is 2-353, or a pharmaceutically acceptable salt thereof.

### Biological Activity

In some cases, the compounds disclosed herein (e.g., compounds of Formula (I), compounds of Formula (I′), compounds of Formula (IA), compounds of Formula (IB), compounds of Formula (IC), compounds of Formula (ID), compounds of Formula (IE), compounds of Formula (IF), compounds listed in Table A, compounds listed in Table B, compounds listed in Table C, compounds listed in Table D, or compounds listed in Table E), or pharmaceutically acceptable salts of the foregoing, have an IC₅₀ value of less than 5 μM, or less than 4 μM, or less than 3 μM, or less than 2 μM, or less than 1 μM, or less than 0.9 μM, or less than 0.7 μM, or less than 0.6 μM, or less than 0.5 μM, or less than 0.4 μM, or less than 0.3 μM, or less than 0.2 μM, or less than 0.1 μM, or less than 0.09 μM, or less than 0.08 μM, or less than 0.07 μM, or less than 0.06 μM, or less than 0.05 μM, or less than 0.04 μM, or less than 0.03 μM, or less than 0.02 μM, or less than 0.01 μM in the coupled exchange assay, which is described in the “BIOLOGICAL EVALUATION” section. In some cases, the compounds disclosed herein, or pharmaceutically acceptable salts thereof, have an IC₅₀ value of less than 1 μM. In some cases, the compounds disclosed herein, and pharmaceutically acceptable salts thereof, have an IC₅₀ value of less than 0.5 μM. In some cases, the compounds disclosed herein, and pharmaceutically acceptable salts thereof, have an IC₅₀ value of less than 0.3 μM. In some cases, the compounds disclosed herein, and pharmaceutically acceptable salts thereof, have an IC₅₀ value of less than 0.1 μM. Also provided herein is a compound of the disclosure, or pharmaceutically acceptable salt thereof, wherein the compound has an IC₅₀ of less than 5 μM in the 2 h coupled exchange assay described herein. Further provided herein is a compound of the disclosure, or pharmaceutically acceptable salt thereof, wherein the compound has an IC₅₀ of less than 3 μM in the 2 h coupled exchange assay described herein. Still further provided herein is a compound of the disclosure, or pharmaceutically acceptable salt thereof, wherein the compound has an IC₅₀ of less than 1 μM in the 2 h coupled exchange assay described herein. Still further provided herein are compounds of the disclosure, or pharmaceutically acceptable salts of the foregoing, having an IC₅₀ of less than 0.5 μM in the 2 h coupled exchange assay described herein. Also provided herein are compounds of the disclosure, or pharmaceutically acceptable salts of the foregoing, having an IC₅₀ of less than 0.1 μM in the 2 h coupled exchange assay described herein. Also provided herein are compounds of the disclosure, or pharmaceutically acceptable salts of the foregoing, having an IC₅₀ of less than 0.05 μM in the 2 h coupled exchange assay described herein. Also provided herein are compounds of the disclosure, or pharmaceutically acceptable salts of the foregoing, having an IC₅₀ of less than 0.04 μM in the 2 h coupled exchange assay described herein. Also provided herein are compounds of the disclosure, or pharmaceutically acceptable salts of the foregoing, having an IC₅₀ of less than 0.03 μM in the 2 h coupled exchange assay described herein. Also provided herein are compounds of the disclosure, or pharmaceutically acceptable salts of the foregoing, having an IC₅₀ of less than 0.02 μM in the 2 h coupled exchange assay described herein. Also provided herein are compounds of the disclosure, or pharmaceutically acceptable salts of the foregoing, having an IC₅₀ of less than 0.01 s M in the 2 h coupled exchange assay described herein.

The foregoing merely summarizes certain aspect of this disclosure and is not intended, nor should it be construed, as limiting the disclosure in any way.

### Formulation and Route of Administration

While it may be possible to administer a compound disclosed herein alone in the uses described, the compound administered normally will be present as an active ingredient in a pharmaceutical composition. Thus, further provided herein is a pharmaceutical composition comprising a compound disclosed herein (e g, a compound of Formula (I), a compound of Formula (I), a compound of Formula (IA), a compound of Formula (IB), a compound of Formula (IC), a compound of Formula (ID), a compound of Formula (IE), a compound of Formula (IF), a compound listed in Table A, a compound listed in Table B, a compound listed in Table C, a compound listed in Table D, or a compound listed in Table E), or a pharmaceutically acceptable salt of any of the foregoing, in combination with one or more pharmaceutically acceptable excipients and, if desired, other active ingredients. See, e.g., Remington: The Science and Practice of Pharmacy, Volume I and Volume II, twenty-second edition, edited by Loyd V. Allen Jr., Philadelphia, PA. Pharmaceutical Press, 2012; Pharmaceutical Dosage Forms (Vol. 1-3), Liberman et al., Eds., Marcel Dekker. New York. NY, 1992; Handbook of Pharmaceutical Excipients (3rd Ed.), edited by Arthur H. Kibbe, American Pharmaceutical Association. Washington, 2000; Pharmaceutical Formulation: The Science and Technology of Dosage Forms (Drug Discovery), first edition, edited by G D Tovey, Royal Society of Chemistry, 2018. In some cases, the pharmaceutical composition described herein comprises a therapeutically effective amount of a compound disclosed herein, or a pharmaceutically acceptable salt thereof.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. In some cases, the pharmaceutical composition is made in the form of a dosage unit containing a particular amount of the active ingredient.

Thus, a further aspect of the disclosure is a pharmaceutical composition comprising one or more of the compounds disclosed herein, or a pharmaceutically acceptable salt thereof, and a pharmaceutically acceptable excipient. Further provided herein is a compound of the disclosure, or a pharmaceutically acceptable salt thereof, or the pharmaceutical composition described herein for use as a medicament.

### Methods of Use

The compounds described herein can covalently bind to cysteine-12 of the GDP-bound form of the G12C-mutant KRAS protein (“KRAS^{G12C}”). In some cases, the compounds described herein can act as potent inhibitors of KRAS^{G12C} by, for example, permanently inactivating the protein. Without intending to be bound by any particular theory, the compounds of the disclosure can, in some cases, inhibit phosphorylation of extracellular signal-regulated (“ERK”), which is a key down-stream effector of KRAS, leading to tumor regression.

Besides being useful for human treatment, the compounds provided herein may be useful for veterinary treatment of companion animals, exotic animals, and farm animals, including mammals, rodents, and the like. For example, animals including horses, dogs, and cats may be treated with compounds provided herein.

### Monotherapy

Another aspect of the disclosure provides methods of using the compounds disclosed herein, or pharmaceutically acceptable salts thereof, or the pharmaceutical compositions of the present disclosure to treat disease conditions, including but not limited to conditions implicated by KRAS^{G12C} mutation (e.g., cancer) See, e.g., U.S. Pat. No. 10,519,146 B2, issued Dec. 31, 2019; specifically, the section from column 198, line 1, to column 201, line 36, which is herewith incorporated by reference.

Without wishing to be bound by any particular theory, the following is noted: sotorasib is a small molecule that—similarly to the compounds disclosed herein—specifically and irreversibly inhibits KRAS^{G12C} (Hong et al., N. Engl. J. Med. 2020, 383, 1207, at 1208). Hong et al report that “[p]reclinical studies showed that [sotorasib]inhibited nearly all detectable phosphorylation of extracellular signal-regulated kinase (ERK), a key down-stream effector of KRAS, leading to durable complete tumor regression in mice bearing KRAS p.G12C tumors.” (id., see also Section entitled “BIOLOGICAL EVALUATION” below, Canon et al., Nature 2019, 575(7781), 217; and Lanman et al., J. Med, Chem. 2020, 63, 52).

Sotorasib was evaluated in a Phase 1 dose escalation and expansion trial with 129 subjects having histologically confirmed, locally advanced or metastatic cancer with the KRAS^{G12C} mutation identified by local molecular testing on tumor tissues, including 59 subjects with non-small cell lung cancer, 42 subjects with colorectal cancer, and 28 subjects with other tumor types (Hong et al., 2020, at page 1208-1209). Hong et al report a disease control rate (95% CI) of 88.1% for non-small cell lung cancer, 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma (Hong et al., 2020, at page 1212 (FIG. A), and Supplementary Appendix (page 59 (FIG. S5) and page 63 (FIG. S6)).

KRAS^{G12C} mutations occur with the alteration frequencies shown in the table below (Cerami et al., Cancer Discov. 2012, 2(5), 401; Gao et al., Science Signaling 2013, 6(269), p 11). For example, the table shows that 11.6% of subjects with non-small cell lung cancer have a cancer, wherein one or more cells express KRAS G12C mutant protein. Accordingly, the compounds provided herein, which specifically and irreversibly bind to KRAS^{G12C} (see Section entitled “BIOLOGICAL EVALUATION” below) are useful for treatment of subjects having a cancer, including, but not limited to the cancers listed in the table below.

| Cancer Type | Alteration Frequency |
|---|---|
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine Tumor | 0.4 |
| Bladder Cancer | 0.4 |
| Myelodysplastic/Myeloproliferative Neoplasms | 0.3 |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

Another aspect of the disclosure provides a compound disclosed herein (e.g., a compound of Formula (I), a compound of Formula (I′), a compound of Formula (IA), a compound of Formula (IB), a compound of Formula (IC), a compound of Formula (ID), a compound of Formula (IE), a compound of Formula (IF), a compound listed in Table A, a compound listed in Table B, a compound listed in Table C, a compound listed in Table D, or a compound listed in Table E)), or a pharmaceutically acceptable salt of any of the foregoing, or a pharmaceutical composition disclosed herein, for use in treating cancer. Yet another aspect of the disclosure provides a compound disclosed herein, or a pharmaceutically acceptable salt thereof, or a pharmaceutical composition disclosed herein, for use in treating cancer, wherein one or more cells express KRAS G12C mutant protein.

Another aspect of the disclosure provides a compound disclosed herein, or a pharmaceutically acceptable salt thereof, or a pharmaceutical composition disclosed herein, in the preparation of a medicament for treating cancer. Yet another aspect of the disclosure provides a compound disclosed herein, or a pharmaceutically acceptable salt thereof, or a pharmaceutical composition described herein, in the preparation of a medicament for treating cancer, wherein one or more cells express KRAS G12C mutant protein.

A further aspect provided by the disclosure is a method of treating cancer in a subject in need thereof, the method comprising administering to the subject a therapeutically effective amount of a compound disclosed herein, or a pharmaceutically acceptable salt thereof, or a pharmaceutical composition disclosed herein. Another aspect of the disclosure is a method of treating cancer in a subject in need thereof, the method comprising administering to the subject a therapeutically effective amount of a compound disclosed herein, or a pharmaceutically acceptable salt thereof, or a pharmaceutical composition of the disclosure, wherein one or more cells express KRAS G12C mutant protein. In some cases, the subject has a cancer that was determined to have one or more cells expressing the KRAS G12C mutant protein prior to administration of the compound or a pharmaceutically acceptable salt thereof.

In some cases, the cancer is metastatic. In some cases, the cancer is non-metastatic. In some cases, the cancer disclosed herein is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor. In some cases, the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, or a solid tumor. In some cases, the cancer is non-small cell lung cancer. In some cases, the cancer is colorectal cancer. In some cases, the cancer is pancreatic cancer. In some cases, the cancer is solid tumor.

### Combination Therapy

The present disclosure also provides methods for combination therapies in which an agent known to modulate other pathways, or other components of the same pathway, or even overlapping sets of target enzymes are used in combination with a compound of the present disclosure (e.g., a compound of Formula (I), a compound of Formula (I′), a compound of Formula (IA), a compound of Formula (IB), a compound of Formula (IC), a compound of Formula (ID), a compound of Formula (IE), a compound of Formula (IF), a compound listed in Table A, a compound listed in Table B, a compound listed in Table C, a compound listed in Table D, or a compound listed in Table E), or a pharmaceutically acceptable salt of any of the foregoing. In one aspect, such therapy includes but is not limited to the combination of one or more compounds of the disclosure with chemotherapeutic agents, therapeutic antibodies, and radiation treatment, to provide a synergistic or additive therapeutic effect. See, e.g., U.S. Pat. No. 10,519,146 B2, issued Dec. 31, 2019, specifically, the sections from column 201 (line 37) to column 212 (line 46) and column 219 (line 64) to column 220 (line 39), which are herewith incorporated by reference.

The compounds of the disclosure can be administered simultaneously, separately, or sequentially with an effective amount of a second compound in an) of the methods described herein. In some cases, the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents. In some cases, the second compound is administered as a pharmaceutically acceptable salt. In some cases, the second compound is administered as a pharmaceutical composition comprising the second compound or a pharmaceutically acceptable salt thereof and a pharmaceutically acceptable excipient.

ATR inhibitors. In some cases, the compounds of the disclosure can be administered simultaneously, separately, or sequentially with an effective amount of an ATR inhibitor in any of the methods described herein. An ATR inhibitor is a compound that targets the ataxia telangiectasia mutated and Rad3-related kinase. Exemplary ATR inhibitors for use in the methods provided herein include, but are not limited to dactolisib, VE-821 (3-Amino-6-(4-(methylsulfonyl)phenyl)-N-phenylpyrazine-2-carboxamide, 3-Amino-6-[4-methylsulfonyl)phenyl]-N-phenyl-2-pyrazinecarboxamide), Torin 2 (9-(6-amino-3-pyridinyl)-1-[3-(trifluoromethyl phenyl]-benzo[h]-1,6-naphthyridin-2(1H)-one), ETP-46464 (α,α-dimethyl-4-[2-oxo-9-(3-quinolinyl)-2H-[1,3]oxazino[5,4-c]quinolin-1(4H)-yl]-benzeneacetonitrile). CGK 733 (α-Phenyl-N-[2,2,2-trichloro-1-[[[(4-fluoro-3-nitrophenyl)amino]thioxomethyl]amino]ethyl]benzeneacetamide), AZ20 (4-[4-[(3R)-3-Methyl-4-morpholinyl]-6-[1-(methylsulfonyl)cyclopropyl]-2-pyrimidinyl]-1H-indole), SKLB-197 ((R)-4-(2-(1H-indol-4-yl)-6-(1-methyl-1H-pyrazol-5-yl)quinazolin-4-yl)-3-methylmorpholine), elimusertib, gartisertib, elimusertib hydrochloride, ceralasertib, and schisandrin B.

Aurora Kinase A Inhibitors. In some cases, the compounds of the disclosure can be administered simultaneously, separately, or sequentially with an effective amount of an Aurora kinase A inhibitor m any of the methods described herein. Exemplary Aurora kinase A inhibitors for use in the methods provided herein include, but are not limited to, alisertib, cenisertib, danusertib, tozasertib, LY3295668 ((2R,4R)-1-[(3-chloro-2-fluorophenyl)methyl]-4-[[3-fluoro-6-[(5-methyl-1H-pyrazol-3-yl)amino]pyridin-2-yl]methyl]-2-methylpiperidine-4-carboxylic acid), ENMD-2076 (6-(4-methylpiperazin-1-yl)-N-(5-methyl-1H-pyrazol-3-yl)-2-[(E)-2-phenylethenyl]pyrimidin-4-amine), TAK-901 (5-(3-ethylsulfonylphenyl)-3,8-dimethyl-N-(1-methylpiperidin-4-yl)-9H-pyrido[2,3-b]indole-7-carboxamide), TT-00420 (4-[9-(2-chlorophenyl)-6-methyl-2,4,5,8,12-pentazatricyclo[8.4.0.03,7]tetradeca-1(14),3,6,8,10,12-hexaen-13-yl]morpholine), AMG 900 (N-[4-[3-(2-aminopyrimidin-4-yl)pyridin-2-yl]oxyphenyl]-4-(4-methylthiophen-2-yl)phthalazin-1-amine), MLN8054 (4-[[9-chloro-7-(2,6-difluorophenyl)-5H-pyrimido[5,4-d][2]benzazepin-2-yl]amino]benzoic acid), PF-03814735 (N-[2-[(1R,8S)-4-[[4-(cyclobutylamino)-5-(trifluoromethyl)pyrimidin-2-yl]amino]-11-azatricyclo[6.2.1.02,7]undeca-2(7),3,5-trien-11-yl]-2-oxoethyl]acetamide), SNS-314 (1-(3-chlorophenyl)-3-[5-[2-(thieno[3,2-d]pyrimidin-4-ylamino)ethyl]-1,3-thiazol-2-yl]urea), CYC116 (4-methyl-5-[2-(4-morpholin-4-ylanilino)pyrimidin-4-yl]-1,3-thiazol-2-amine), TAS-119, BI 811283, and TTP607.

AKT Inhibitors. In some cases, the compounds of the disclosure can be administered simultaneously, separately, or sequentially with an effective amount of an AKT inhibitor in any of the methods described herein. Exemplary AKT inhibitors for use in the methods provided herein include, but are not limited to, afuresertib, capivasertib, ipatasertib, uprosertib, BAY1125976 (2-[4-(1-aminocyclobutyl)phenyl]-3-phenylimidazo[1,2-b]pyridazine-6-carboxamide), ARQ 092 (3-[3-[4-(1-aminocyclobutyl)phenyl]-5-phenylimidazo[4,5-b]pyridin-2-yl]pyridin-2-amine), MK2206 (8-[4-(1-aminocyclobutyl)phenyl]-9-phenyl-2H-[1,2,4]triazolo[3,4-f[1,6]naphthyridin-3-one), SR13668 (indolo[2,3-b]carbazole-2,10-dicarboxylic acid, 5,7-dihydro-6-methoxy-, 2,10-diethyl ester), ONC201 (11-benzyl-7-[(2-methylphenyl)methyl]-2,5,7,11-tetrazatricyclo[7.4.0.02,6]trideca-1(9),5-dien-8-one), ARQ 751 (N-(3-aminopropyl)-N-[(1R)-1-(3-anilino-7-chloro-4-oxoquinazolin-2-yl)but-3-ynyl]-3-chloro-2-fluorobenzamide), RX-0201, and LY2780301.

Arginase Inhibitors. In some cases, the compounds of the disclosure can be administered simultaneously, separately, or sequentially with an effective amount of an arginase inhibitor in any of the methods described herein. Exemplary arginase inhibitors for use in the methods provided herein include, but are not limited to, numidargistat and CB 280.

CDK 2 Inhibitors. In some cases, the compounds of the disclosure can be administered simultaneously, separately, or sequentially with an effective amount of a CDK 2 inhibitor in any of the methods described herein. The term “CDK 2” as used herein refers to cyclin dependent kinases (“CDK”) 2, which is a member of the mammalian serine/threonine protein kinases. The term “CDK 2 inhibitor” as used herein refers to a compound that is capable of negatively modulating or inhibiting all or a portion of the enzymatic activity of CDK 2. Exemplary CDK 2 inhibitors for use in the methods provided herein include, but are not limited to, flavopiridol, roscovitine, dinaciclib, milciclib, meriolin, variolin, AZD5438 (4-[2-Methyl-1-(1-methylethyl)-1H-imidazol-5-yl]-N-[4-(methylsulfonyl)phenyl]-2-pyrimidinamine), roniciclib, SNS-032 (N-[5-[[[5-(1,1-Dimethylethyl)-2-oxazolyl]methyl]thio]-2-thiazolyl]-4-piperidinecarboxamide).

CDK4/6 Inhibitors. In some cases, the compounds of the disclosure can be administered simultaneously, separately, or sequentially with an effective amount of a CDK4/6 inhibitor in any of the methods described herein. The term “CDK 4/6” as used herein refers to cyclin dependent kinases (“CDK”) 4 and 6, which are members of the mammalian serine/threonine protein kinases. The term “CDK 4/6 inhibitor” as used herein refers to a compound that is capable of negatively modulating or inhibiting all or a portion of the enzymatic activity of CDK 4 and/or 6. Exemplary CDK 4/6 inhibitors for use in the methods provided herein include, but are not limited to, abemaciclib, palbociclib, ribociclib, trilaciclib, and PF-06873600 ((pyrido[2,3-d]pyrimidin-7(8H)-one, 6-(difluoromethyl)-8-[(1R,2R)-2-hydroxy-2-methylcyclopentyl]-2-[[1-(methylsulfonyl)-4-piperidinyl]amino]). In some cases, the CDK4/6 inhibitor is palbociclib.

ErbB Family Inhibitors. In some cases, the compounds of the disclosure can be administered simultaneously, separately, or sequentially with an effective amount of an ErbB family inhibitor in any of the methods described herein. The term “ErbB family” as used herein refers to a member of a mammalian transmembrane protein tyrosine kinase family including: ErbB1 (EGFR HER1), ErbB2 (HER2), ErbB3 (HER3), and ErbB4 (HER4). The term “ErbB family inhibitor” as used herein refers to an agent, e.g., a compound or antibody, that is capable of negatively modulating or inhibiting all or a portion of the activity of at least one member of the ErbB family. The modulation or inhibition of one or more ErbB tyrosine kinase may occur through modulating or inhibiting kinase enzymatic activity of one or more ErbB family member or by blocking homodimerization or heterodimerization of ErbB family members. In some cases, the ErbB family inhibitor is an EGFR inhibitor, e.g., an anti-EGFR antibody. Exemplary anti-EGFR antibodies for use in the methods provided herein include, but are not limited to, zalutumumab, nimotuzumab, matuzumab, necitumumab, panitumumab, and cetuximab. In some cases, the anti-EGFR antibody is cetuximab. In some cases, the anti-EGFR antibody is panitumumab. In some cases, the ErbB family inhibitor is a HER2 inhibitor, e.g., an anti-HER2 antibody. Exemplary anti-HER-2 antibodies for use in the methods provided herein include, but are not limited to, pertuzumab, trastuzumab, and trastuzumab emtansine. In some cases, the ErbB family inhibitor is a HER3 inhibitor, e.g., an ani-HER3 antibody, such as HMBD-001 (Hummingbird Bioscience). In some cases, the ErbB family inhibitor is a combination of an anti-EGFR antibody and anti-HER2 antibody. In some cases, the ErbB family inhibitor is an irreversible inhibitor, Exemplary irreversible ErbB family inhibitors for use in the methods provided herein include, but are not limited to, afatinib, dacomitinib, canertinib, poziotinib, AV 412 ((N-[4-[(3-chloro-4-fluorophenyl)amino]-7-[3-methyl-3-(4-methyl-1-piperazinyl)-1-butyn-1-yl]-6-quinazolinyl]-2-propenamide)), PF 6274484 ((N-[4-[(3-chloro-4-fluorophenyl)amino]-7-methoxy-6-quinazolinyl]-2-propenamide), and HKI 357 ((E)-N-[4-[3-chloro-4-[(3-fluorophenyl)methoxy]anilino]-3-cyano-7-ethoxyquinolin-6-yl]-4-(dimethylamino)but-2-enamide). In some cases, the irreversible ErbB family inhibitor is afatinib. In some cases, the irreversible ErbB family inhibitor is dacomitinib. In some cases, the ErbB family inhibitor is a reversible inhibitor, Exemplary reversible ErbB family inhibitors for use in the methods provided herein include, but are not limited to erlotinib, gefitinib, sapitinib, varlitinib, tarloxotinib. TAK-285 (N-(2-(4-((3-chloro-4-(3-(trifluoromethyl)phenoxy)phenyl)amino)-5H-pyrrolo[3,2-d]pyrimidin-5-yl)ethyl)-3-hydroxy-3-methylbutanamide), AEE788((S)-6-(4-((4-ethylpiperazin-1-yl)methyl)phenyl)-N-(1-phenylethyl)-7H-pyrrolo[2,3-d]pyrimidin-4-amine), BMS 599626 ((3S)-3-morpholinylmethyl-[4-[[1-[(3-fluorophenyl)methyl]-1H-indazol-5-yl]amino]-5-methylpyrrolo[2,1-f][1,2,4]triazin-6-yl]-carbamate), and GW 583340 (N-[3-chloro-4-[(3-fluorophenyl)methoxy]phenyl]-6-[2-[(2-methylsulfonylethylamino)methyl]-1,3-thiazol-4-yl]quinazolin-4-amine). In some cases, the reversible ErbB family inhibitor is sapitinib. In one embodiment, the reversible ErbB family inhibitor is tarloxotinib.

ERK Inhibitors. In some cases, the compounds of the disclosure can be administered simultaneously, separately, or sequentially with an effective amount of an ERK inhibitor in any of the methods described herein. Exemplary ERK inhibitors for use in the methods provided herein include, but are not limited to, ulixertinib, ravoxertinib, CC-90003 (N-[2-[[2-[(2-methoxy-5-methylpyridin-4-yl)amino]-5-(trifluoromethyl)pyrimidin-4-yl]amino]-5-methylphenyl]prop-2-enamide), LY3214996 (6,6-dimethyl-2-[2-[(2-methylpyrazol-3-yl)amino]pyrimidin-4-yl]-5-(2-morpholin-4-ylethyl)thieno[2,3-c]pyrrol-4-one), KO-947 (1,5,6,8-tetrahydro-6-(phenylmethyl)-3-(4-pyridinyl)-7H-pyrazolo[4,3-g]quinazolin-7-one), ASTX029, LTT462, and JSI-1187.

FAK Inhibitors. In some cases, the compounds of the disclosure can be administered simultaneously, separately, or sequentially with an effective amount of a FAK inhibitor in any of the methods described herein. Exemplary FAK inhibitors for use in the methods provided herein include, but are not limited to, GSK2256098 (2-[[5-chloro-2-[(5-methyl-2-propan-2-ylpyrazol-3-yl)amino]pyridin-4-yl]amino]-N-methoxybenzamide), PF-00562271 (N-methyl-N-[3-[[[2-[(2-oxo-1,3-dihydroindol-5-yl)amino]-5-(trifluoromethyl)pyrimidin-4-yl]amino]methyl]pyridin-2-yl]methanesulfonamide), VS-4718 (2-[[2-(2-methoxy-4-morpholin-4-ylanilino)-5-(trifluoromethyl)pyridin-4-yl]amino]-N-methylbenzamide), and APG-2449.

FGFR Inhibitors. In some cases, the compounds of the disclosure can be administered simultaneously, separately, or sequentially with an effective amount of an FGFR inhibitor in any of the methods described herein. Exemplary FGFR inhibitors for use in the methods provided herein include, but are not limited to, futibatinib, pemigatinib. ASP5878 (2-[4-[[5-[(2,6-difluoro-3,5-dimethoxyphenyl)methoxy]pyrimidin-2-yl]amino]pyrazol-1-yl]ethanol), AZD4547 (N-[5-[2-(3,5-dimethoxyphenyl)ethyl]-1H-pyrazol-3-yl]-4-[(3S,5R)-3,5-dimethylpiperazin-1-yl]benzamide), debio 1347 ([5-amino-1-(2-methyl-3H-benzimidazol-5-yl)pyrazol-4-yl]-(1H-indol-2-yl)methanone), INCB062079, H3B-6527 (N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide), ICP-105, CPL304110, HMPL-453, and HGS1036.

Glutaminase Inhibitors. In some cases, the compounds of the disclosure can be administered simultaneously, separately, or sequentially with an effective amount of a glutaminase inhibitor in any of the methods described herein. Exemplary glutaminase inhibitors for use in the methods provided herein include, but are not limited to, telaglenastat, IPN60090, and OP 330.

IGF-1R Inhibitors. In some cases, the compounds of the disclosure can be administered simultaneously, separately, or sequentially with an effective amount of an IGF-1R inhibitor in any of the methods described herein. Exemplary IGF-1R inhibitors for use in the methods provided herein include, but are not limited to, cixutumumab, dalotuzumab, linsitinib, ganitumab, robatumumab, BMS-754807 ((2S)-1-[4-[(5-cyclopropyl-1H-pyrazol-3-yl)amino]pyrrolo[2,1-f][1,2,4]triazin-2-yl]-N-(6-fluoropyridin-3-yl)-2-methylpyrrolidine-2-carboxamide), KW-2450 (N-[5-[[4-(2-hydroxyacetyl)piperazin-1-yl]methyl]-2-[(E)-2-(1H-indazol-3-yl)ethenyl]phenyl]-3-methylthiophene-2-carboxamide), PL225B, AVE1642, and BIIB022.

KIF18A Inhibitors. In some cases, the compounds of the disclosure can be administered simultaneously, separately, or sequentially with an effective amount of a KIF18A inhibitor in any of the methods described herein. Exemplary KIF18A inhibitors for use in the methods provided herein include, but are not limited to, the inhibitors disclosed in US 2020/0239441, WO 2020/132649, WO 2020/132651, and WO 20201132653, each of which is herewith incorporated by reference in its entirety. In some cases, the KIF18A inhibitor is sovilnesib (AMG 650).

MAT2A inhibitors. In some cases, the compounds of the disclosure can be administered simultaneously, separately, or sequentially with an effective amount of a MAT2A inhibitor in any of the methods described herein. An MAT2A inhibitor is a compound that inhibits methionine adenosyltransferase II alpha. An exemplary MAT2A inhibitor for use in the methods provided herein is AG 270 (3-(cyclohex-1-en-1-yl)-6-(4-methoxyphenyl)-2-phenyl-5-(pyridin-2-ylamino)pyrazolo[1,5-a]pyrimidin-7(4H)-one).

MCL-1 Inhibitors. In some cases, the compounds of the disclosure can be administered simultaneously, separately, or sequentially with an effective amount of a MCL-1 inhibitor in any of the methods described herein. Exemplary MCL-1 inhibitors for use in the methods provided herein include, but are not limited to, murizatoclax, tapotoclax, AZD 5991 ((3aR)-5-chloro-2,11,12,24,27,29-hexahydro-2,3,24,33-tetramethyl-22H-9,4,8-(methaniminomethano)-14,20:26,23-dimethano-10H,20H-pyrazolo[4,3-1][2,5,22,18,19]benzoxadithiadiazacyclohexacosine-32-carboxylic acid), MIK 665 ((αR)-α-[[(5S)-5-[3-Chloro-2-methyl-4-[2-(4-methyl-1-piperazinyl)ethoxy]phenyl]-6-(4-fluorophenyl)thieno[2,3-d]pyrimidin-4-yl]oxy]-2-[[2-(2-methoxyphenyl)-4-pyrimidinyl]methoxy]benzenepropanoic acid), and ABBV-467. In some cases, the MCL-1 inhibitor is murizatoclax. In some cases, the MCL-1 inhibitor is tapotoclax.

MEK Inhibitors. In some cases, the compounds of the disclosure can be administered simultaneously, separately, or sequentially with an effective amount of a MEK inhibitor in any of the methods described herein. Exemplary MEK inhibitors for use in the methods provided herein include, but are not limited to, trametinib, cobimetinib, selumetinib, pimasertib, refametinib, PD-325901 (N-[(2R)-2,3-dihydroxypropoxy]-3,4-difluoro-2-(2-fluoro-4-iodoanilino)benzamide), AZD8330 (2-(2-fluoro-4-iodoanilino)-N-(2-hydroxyethoxy)-1,5-dimethyl-6-oxopyridine-3-carboxamide), GDC-0623 (5-(2-fluoro-4-iodoanilino)-N-(2-hydroxyethoxy)imidazo[1,5-a]pyridine-6-carboxamide), R⁰⁴⁹⁸⁷⁶⁵⁵ (3,4-difluoro-2-(2-fluoro-4-iodoanilino)-N-(2-hydroxyethoxy)-5-[(3-oxooxazinan-2-yl)methyl]benzamide), TAK-733 (3-[(2R)-2,3-dihydroxypropyl]-6-fluoro-5-(2-fluoro-4-iodoindolo)-8-methylpyrido[2,3-d]pyrimidine-4,7-dione), PD0325901 (N-[(2R)-2,3-dihydroxypropoxy]-3,4-difluoro-2-(2-fluoro-4-iodoanilino)benzamide), CI-1040 (2-(2-chloro-4-iodophenylamino)-N-(cyclopropylmethoxy)-3,4-difluorobenzamide), PD318088 (5-bromo-N-(2,3-dihydroxypropoxy)-3,4-difluoro-2-(2-fluoro-4-iodophenylamino)benzamide, PD98059 (2-(2-amino-3-methoxyphenyl)-4H-chromen-4-one), PD334581 (N-[5-[3,4-Difluoro-2-[(2-fluoro-4-iodophenyl)amino]phenyl]-1,3,4-oxadiazol-2-yl]-4-morpholineethanamine), FCN-159, CS3006, HL-085, SHR 7390, and WX-554. In some cases, the MEK inhibitor is trametinib.

mTOR Inhibitors. In some cases, the compounds of the disclosure can be administered simultaneously, separately, or sequentially with an effective amount of a mTOR inhibitor in any of the methods described herein. Exemplary mTOR inhibitors for use in the methods provided herein include, but are not limited to, everolimus, rapamycin, zotarolimus (ABT-578), ridaforolimus (deforolimus. MK-8669), sapanisertib, buparlisib, pictilisib, vistusertib, dactolisib, Torin-1 t 1-(4-(4-propionylpiperazin-1-yl)-3-(trifluoromethyl)cyclohexyl)-9-(quinolin-3-yl)benzo[h][1,6]naphthyridin-2(1H)-one), GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), and VS-5584 (SB2343, (5-(8-methyl-2-morpholin-4-yl-9-propan-2-ylpurin-6-yl)pyrimidin-2-amine). In some cases, the mTOR inhibitor is everolimus.

PARP inhibitors. In some cases, the compounds of the disclosure can be administered simultaneously, separately, or sequentially with an effective amount of a PARP inhibitor in any of the methods described herein. A PARP inhibitor is a compound that targets poly(adenosine diphosphate)-ribose polymerase. The term PARP inhibitors encompasses PARP1, PARP2, and PARP3 inhibitors. Exemplary PARP inhibitors for use in the methods provided herein include, but are not limited to, olaparib, rucaparib, rucaparib camsylate, niraparib, niraparib tosylate, talazoparib. AG-1461, A-966492, PJ34 HCl, niraparib, UPF 1069, ME0328, venadaparib, AZD5305, DR2313, BYK204165, pamiparib, NMS-P118, and NU 1025.

PD-1 Inhibitors. In some cases, the compounds of the disclosure can be administered simultaneously, separately, or sequentially with an effective amount of a PD-1 inhibitor in any of the methods described herein. Exemplary PD-1 inhibitors for use in the methods provided herein include, but are not limited to, pembrolizumab, nivolumab, cemiplimab, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, and the anti-PD-1 antibody as described in U.S. Pat. No. 10,640,504 B2 (the “Anti-PD-1 Antibody A,” column 66, line 56 to column 67, line 24 and column 67, lines 54-57), which is incorporated herein by reference. In some cases, the PD-1 inhibitor is pembrolizumab. In some cases, the PD-1 inhibitor is the Anti-PD-1 Antibody A.

PD-L1 Inhibitors. In some cases, the compounds of the disclosure can be administered simultaneously, separately, or sequentially with an effective amount of a PD-L1 inhibitor in any of the methods described herein. Exemplary PD-L1 inhibitors for use in the methods provided herein include, but are not limited to, atezolizumab, avelumab, durvalumab, ZKAB001, TG-1501, SHR-1316, MSB2311, MDX-1105, KN035, TMC-001, HLX20, FAZ053, CSI001, CK-301, CBT-502, BGB-A333, BCD-135, and A167. In some cases, the PD-L1 inhibitor is atezolizumab.

PI3K Inhibitors. In some cases, the compounds of the disclosure can be administered simultaneously, separately, or sequentially with an effective amount of a PI3K inhibitor in any of the methods described herein. Exemplary PI3K inhibitors for use in the methods provided herein include, but are not limited to, idelalisib, copanlisib, duvelisib, alpelisib, taselisib, perifosine, buparlisib, umbralisib, pictilisib, dactolisib, voxtalisib, sonolisib, tenalisib, serabelisib, acalisib, CUDC-907 (N-hydroxy-2-[[2-(6-methoxypyridin-3-yl)-4-morpholin-4-ylthieno[3,2-d]pyrimidin-6-yl]methyl-methylamino]pyrimidine-5-carboxamide), ME-401 (N-[2-methyl-1-[2-(1-methylpiperidin-4-yl)phenyl]propan-2-yl]-4-(2-methylsulfonylbenzimidazol-1-yl)-6-morpholin-4-yl-1,3,5-triazin-2-amine). IPI-549 (2-amino-N-[(1S)-1-[8-[2-(1-methylpyrazol-4-yl)ethynyl]-1-oxo-2-phenylisoquinoline-3-yl]ethyl]pyrazolo[1,5-a]pyrimidine-3-carboxamide), SF1126 ((2S)-2-[[(2S)-3-carboxy-2-[[2-[[(2S)-5-(diaminomethylideneamino)-2-[[4-oxo-4-[[4-(4-oxo-8-phenylchromen-2-yl)morpholin-4-ium-4-yl]methoxyIbutanoyljaminolpentanoyllammolacetylaminolpropanoyljaminoI-3-hydroxypropanoate), XL147 (N-[3-(2,1,3-benzothiadiazol-5-ylamino)quinoxalin-2-yl]-4-methylbenzenesulfonamide), GSK1059615 ((5Z)-5-[(4-pyridin-4-ylquinolin-6-yl)methylidene]-1,3-thiazolidine-2,4-dione), and AMG 319 (N-[(1S)-1-(7-fluoro-2-pyridin-2-ylquinolin-3-yl)ethyl]-7H-purin-6-anine).

PRMT5 Inhibitors. In some cases, the compounds of the disclosure can be administered simultaneously, separately, or sequentially with an effective amount of a PRMT5 inhibitor in any of the methods described herein. A PRMT5 inhibitor in a compound that inhibits protein arginine methyltransferase 5. The term “PRMT5 inhibitor” includes MTA-cooperative PRMT5 inhibitors Exemplary PRMT5 inhibitors for use in the methods provided herein include, but are not limited to, pemrametostat (6-[(1-acetylpiperidin-4-yl)amino]-N-[(2S)-3-(3,4-dihydro-1H-isoquinolin-2-yl)-2-hydroxypropyl]pyrimidine-4-carboxamide), GSK3203591 (2-(Cyclobutylamino)-N-[(2S)-3-(3,4-dihydro-2(1H)-isoquinolinyl)-2-hydropropyl]-4-pyridinecarboxamide dihydrochloride)), LLY-283 ((R)-5′-phenyl-7-deazaadenosine; 6-amino-9-[(R)-5′-phenyl(ribofuranosyl)]-7-deazapurine, (2R,3R,4S,5R)-2-(4-Amino-7H-pyrrolo[2,3-d]pyrimidin-7-yl)-1)-5-((R)-hydroxy(phenyl)methyl)tetrahydrofuran-3,4-diol), PRT 811, and MRTX1719 (2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile).

Raf Kinase Inhibitors. In some cases, the compounds of the disclosure can be administered simultaneously, separately, or sequentially with an effective amount of a Raf kinase inhibitor in any of the methods described herein. The term “RAF kinase” as used herein refers to a member of a mammalian serine/threonine kinases composed of three isoforms (C-Raf, B-Raf and A-Raf) and includes homodimers of each isoform as well as heterodimers between isoforms, e.g., C-Raf/B-Raf heterodimers. The term “Raf kinase inhibitor,” as used herein refers to a compound that is capable of negatively modulating or inhibiting all or a portion of the enzymatic activity of one or more member of the Raf family kinases, or is capable of disrupting Raf homodimer or heterodimer formation to inhibit activity. In some cases, the Raf kinase inhibitor includes, but is not limited to, encorafenib, sorafenib, lifirafenib, vemurafenib, dabrafenib, PLX-8394 (N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide), Raf-709 (N-(2-methyl-5,-morpholino-6′-((tetrahydro-2H-pyran-4-yl)oxy)-[3,3′-bipyridin]-5-yl)-3-(trifluoromethyl)benzamide), LXH254 (N-(3-(2-(2-hydroxyethoxy)-6-morpholinopyridin-4-yl)-4-methylphenyl)-2-(trifluoromethyl)isonicotinamide), LY3009120 (1-(3,3-dimethylbutyl)-3-(2-fluoro-4-methyl-5-(7-methyl-2-(methylamino)pyrido[2,3-d]pyrimidin-6-yl)phenyl)urea), Tak-632 (N-(7-cyano-6-(4-fluoro-3-(2-(3-(trifluoromethyl)phenyl)acetamido)phenoxy)benzo[d]thiazol-2-yl)cyclopropanecarboxamide), CEP-32496 (1-(3-((6,7-dimethoxyquinazolin-4-yl)oxy)phenyl)-3-(5-(1,1,1-trifluoro-2-methylpropan-2-yl)isoxazol-3-yl)urea), CCT196969 (1-(3-(tert-butyl)-1-phenyl-H-pyrazol-5-yl)-3-(2-fluoro-4-((3-oxo-3,4-dihydropyrido[2,3-b]pyrazin-8-yl)oxy)phenyl)urea), and RO5126766 (N-[3-fluoro-4-[[4-methyl-2-oxo-7-(2-pyrimidinyloxy)-2H-1-benzopyran-3-yl]methyl]-2-pyridinyl]-N′-methyl-sulfamide). In some cases, the Raf kinase inhibitor is encorafenib. In some cases, the Raf kinase inhibitor is sorafenib. In some cases, the Raf kinase inhibitor is lifirafenib.

SHP2 Inhibitors. In some cases, the compounds of the disclosure can be administered simultaneously, separately, or sequentially with an effective amount of a SHP2 inhibitor in any of the methods described herein. Exemplary SHP2 inhibitors for use in the methods provided herein include, but are not limited to, SHP-099 (6-(4-amino-4-methylpiperidin-1-yl)-3-(2,3-dichlorophenyl)pyrazin-2-amine dihydrochloride), RMC-4550 ([3-[(3S,4S)-4-amino-3-methyl-2-oxa-8-azaspiro[4.5]decan-8-yl]-6-(2,3-dichlorophenyl)-5-methylpyrazin-2-yl]methanol), TNO155, (3S,4S)-8-[6-amino-5-(2-amino-3-chloropyridin-4-yl)sulfanylpyrazin-2-yl]-3-methyl-2-oxa-8-azaspiro[4.5]decan-4-amine), and RMC-4630 (Revolution Medicine: vociprotafib (RMC-4630; 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol). In some cases, the SHP inhibitor for use in the methods provided herein is RMC-4630 (vociprotafib, Revolution Medicine). In some cases, exemplary SHP2 inhibitors for use in the methods provided herein include, but are not limited to, 3-[(1R,3R)-1-amino-3-methoxy-8-azaspiro[4.5]dec-8-yl]-6-(2,3-dichlorophenyl)-5-methyl-2-pyrazinemethanol (CAS 2172651-08-8), 3-[(3S,4S)-4-amino-3-methyl-2-oxa-8-azaspiro[4.5]dec-8-yl]-6-[(2,3-dichlorophenyl)thio]-5-methyl-2-pyrazinemethanol (CAS 2172652-13-8), 3-[(3S,4S)-4-amino-3-methyl-2-oxa-8-azaspiro[4.5]dec-8-yl]-6-[[3-chloro-2-(3-hydroxy-1-azetidinyl)-4-pyridinyl]thio]-5-methyl-2-pyrazinemethanol (CAS 2172652-38-7), and 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol (CAS 217265248-9). In some cases, exemplary SHP2 inhibitors for use in the methods provided herein include, but are not limited to, 1-[5-(2,3-dichlorophenyl)-6-methylimidazo[1,5-a]pyrazin-8-yl]-4-methyl-4-piperidinamine (CAS 2240981-75-1), (1R)-8-[5-(2,3-dichlorophenyl)-6-methylimidazo[1,5-a]pyrazin-8-yl]-8-azaspiro[4.5]decan-1-amine (CAS 2240981-78-4). (3S,4S)-8-[7-(2,3-dichlorophenyl)-6-methylpyrazolo[1,5-a]pyrazin-4-yl]-3-methyl-2-oxa-8-azaspiro[4.5]decan-4-amine (CAS 2240982-45-8), (3S,4S)-8-[7-[(2-amino-3-chloro-4-pyridinyl)thiolpyrazolo[1,5-a]pyrazin-4-yl]-3-methyl-2-oxa-8-azaspiro[4.5]decan-4-amine (CAS 2240982-57-2), 4-[(3S,4S)-4-amino-3-methyl-2-oxa-8-azaspiro[4.5]dec-8-yl]-7-(2,3-dichlorophenyl)-6-methyl-pyrazolo[1,5-a]pyrazine-2-methanol (CAS 2240982-69-6), 7-[(2-amino-3-chloro-4-pyridinyl)thio]-4-[(3S,4S)-4-amino-3-methyl-2-oxa-8-azaspiro[4.5]dec-8-yl]-6-methyl-pyrazolo[1,5-a]pyrazine-2-methanol (CAS 2240982-73-2), and (3S,4S)-8-[7-[(2-amino-3-chloro-4-pyridinyl)thio]-6-methylpyrazolo[1,5-a]pyrazin-4-yl]-3-methyl-2-oxa-8-azaspiro[4.5]decan-4-amine (CAS 2240982-77-6). In some cases, the SHP inhibitor for use in the methods provided herein is (1R)-8-[5-(2,3-dichlorophenyl)-6-methylimidazo[1,5-a]pyrazin-8-yl]-8-azaspiro[4.5]decan-1-amine (CAS 2240981-78-4). In some cases, exemplary SHP2 inhibitors for use in the methods provided herein include, but are not limited to 3-[(1R)-1-amino-8-azaspiro[4.5]dec-8-yl]-6-(2,3-dichlorophenyl)-5-hydroxy-2-pyridinemethanol (CAS 2238840-54-3), 3-[(1R)-1-amino-8-azaspiro[4.5]dec-8-yl]-6-[(2,3-dichlorophenyl)thio]-5-hydroxy-2-pyridinemethanol (CAS 2238840-56-5), 5-[(1R)-1-amino-8-azaspiro[4.5]dec-8-yl]-2-(2,3-dichlorophenyl)-3-pyridinol (CAS 2238840-58-7), 3-[(1R)-1-amino-8-azaspiro[4.5]dec-8-yl]-6-(2,3-dichlorophenyl)-5-methyl-2-pyridinemethanol (CAS 2238840-60-1), (1R)-8-16-(2,3-dichlorophenyl)-5-methyl-3-pyridinyl]-8-azaspiro[4.5]decan-1-amine (CAS 2238840-62-3), 3-(1R)-1-amino-8-azaspiro[4.5]dcc-8-yl]-6-[(2,3-dichlorophenyl)thio]-5-methyl-2-pyridinemethanol (CAS 2238840-63-4), (1R)-8-[6-[(2,3-dichlorophenyl)thio]-5-methyl-3-pyridinyl]-8-azaspiro[4.5]decan-1-amine (CAS 2238840-64-5), 5-(4-amino-4-methyl-1-piperidinyl)-2-[(2,3-dichlorophenyl)thio]-3-pyridinol (CAS 2238840-65-6), 5-[(1R)-1-amino-8-azaspiro[4.5]dec-8-yl]-2-[(2,3-dichlorophenyl)thio]-3-pyridinol (CAS 2238840-66-7), 6-[(2-amino-3-chloro-4-pyridin)₁)thio]-3-[(3S,4S)-4-amino-3-methyl-2-oxa-8-azaspiro[4.5]dec-8-yl]-5-hydroxy-2-pyridinemethanol (CAS 2238840-67-8), 3-(4-amino-4-methyl-1-piperidinyl)-6-(2,3-dichlorophenyl)-5-hydroxy-2-pyridinemethanol (CAS 2238840-68-9), 3-[(3S,4S)-4-amino-3-methyl-2-oxa-8-azaspiro[4.5]dec-8-yl]-6-(2,3-dichlorophenyl)-5-methyl-2-pyridinemethanol (CAS 2238840-69-0), 6-[(2-amino-3-chloro-4-pyridinyl)thio]-3-[(3S,4S)-4-amino-3-methyl-2-oxa-8-azaspiro[4.5]dec-8-yl]-5-methyl-2-pyridinemethanol (CAS 2238840-70-3), 3-(4-amino-4-methyl-1-piperidinyl)-6-(2,3-dichlorophenyl)-5-methyl-2-pyridinemethanol (CAS 2238840-71-4), 6-[(2-amino-3-chloro-4-pyridinyl)thio]-3-(4-amino-4-methyl-1-piperidinyl)-2-pyridinemethanol (CAS 2238840-72-5), 5-[(2-amino-3-chloro-4-pyridinyl)thio]-2-[(3S,4S)-4-amino-3-methyl-2-oxa-8-azaspiro[4.5]dec-8-yl]-6-methyl-3-pyridinemethanol (CAS 2238840-73-6), 2-[(3S,4S)-4-amino-3-methyl-2-oxa-8-azaspiro[4.5]dec-8-yl]-5-(2,3-dichlorophenyl)-6-methyl-3-pyridinemethanol (CAS 2238840-74-7), 3-[(3S,4S)-4-amino-3-methyl-2-oxa-8-azaspiro[4.5]dec-8-yl]-6-(2,3-dichlorophenyl)-5-hydroxy-2-pyridinemethanol (CAS 2238840-75-8), and 2-[(2-amino-3-chloro-4-pyridyl)sulfanyl]-5-[(3S,4S)-4-amino-3-methyl-2-oxa-8-azaspiro[4.5]decan-8-yl]-6-(hydroxymethyl)pyridin-3-ol. In some cases, the SHP inhibitor for use in the methods provided herein is 3-[(1R)-1-amino-8-azaspiro[4.5]dec-8-yl]-6-[(2,3-dichlorophenyl)thio]-5-hydroxy-2-pyridinemethanol (CAS 2238840-56-5). In some cases, the SHP2 inhibitor for use in the methods provided herein is an inhibitor disclosed in U.S. Pat. No. 10,590,090 B2, US 2020/017517 A1, US 2020/017511 A1, WO 2019/075265 A1, or WO 2021/142026 A1, each of which is herewith incorporated by reference in its entirety.

SOS1 Inhibitors. In some cases, the compounds of the disclosure can be administered simultaneously, separately, or sequentially with an effective amount of a SOS1 inhibitor in any of the methods described herein. Exemplary SOS1 inhibitors for use in the methods provided herein include, but are not limited to, BI 3406 (N-[(1R)-1-[3-amino-5-(trifluoromethyl)phenyl]ethyl]-7-methoxy-2-methyl-6-[(3S)-oxolan-3-yl]oxoquinazolin-4-amine), BI 1701963, AST-NS2102, MRTX-0902 ((R)-2-methyl-3-(1-((4-methyl-7-morpholinopyrido[3,4-d]pyridazin-J-yl)amino)ethyl)benzonitrile), ERAS-9, RMC-5845, HM-99462, and GH-52.

Src Kinase Inhibitors. In some cases, the compounds of the disclosure can be administered simultaneously, separately, or sequentially with an effective amount of a Src kinase inhibitor in any of the methods described herein. The term “Src kinase” as used herein refers to a member of a mammalian nonreceptor tyrosine kinase family including: Src, Yes, Fyn, and Fgr (SrcA subfamily); Lck, Hck, Blk, and Lyn (SrcB subfamily), and Frk subfamily. The term “Src kinase inhibitor” as used herein refers to a compound that is capable of negatively modulating or inhibiting all or a portion of the enzymatic activity of one or more member of the Src kinases. Exemplary Src kinase inhibitors for use in the methods provided herein include, but are not limited to, dasatinib, ponatinib, vandetanib, bosutinib, saracatinib, KX2-391 (N-benzyl-2-(5-(4-(2-morpholinoethoxy)phenyl)pyridin-2-yl)acetamide), SU6656 ((Z)—N,N-dimethyl-2-oxo-3-((4,5,6,7-tetrahydro-1H-indol-2-yl)methylene)indoline-5-sulfonamide), PP 1 (1-(tert-butyl)-3-(p-tolyl)-1H-pyrazolo[3,4-d]pyrimidin-4-amine), WH-4-023 (2,6-dimethylphenyl(2,4-dimethoxyphenyl)(2-((4-(4-methylpiperazin-1-yl)phenyl)amino)pyrimidin-4-yl)carbamate), and KX-01 (N-benzyl-2-(5-(4-(2-morpholinoethoxy)phenyl)pyridin-2-yl)acetamide). In some cases, the Src kinase inhibitor is dasatinib. In some cases, the Src kinase inhibitor is saracatinib. In some cases, the Src kinase inhibitor is ponatinib. In some cases, the Src kinase inhibitor is vandetanib. In some cases, the Src kinase inhibitor is KX-01.

Chemotherapeutic Agents. In some cases, the compounds of the disclosure can be administered simultaneously, separately, or sequentially with an effective amount of one or more chemotherapeutic agents in any of the methods described herein. Exemplary chemotherapeutic agents for use in the methods provided herein include, but are not limited to, leucovorin calcium (calcium folinate), 5-fluorouracil, irinotecan, oxaliplatin, cisplatin, carboplatin, pemetrexed, docetaxel, paclitaxel, gemcitabine, vinorelbine, chlorambucil, cyclophosphamide, and methotrexate.

### Definitions and General Terminology

The following definitions are provided to assist in understanding the scope of this disclosure.

Unless otherwise indicated, all numbers expressing quantities of ingredients, reaction conditions, and so forth used in the specification or claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropisomers. Accordingly, the scope of the present disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of the structure. If the stereochemistry of a structure or a portion of a structure is indicated with, for example, bold or dashed lines, the structure or portion of the structure is to be interpreted as encompassing only the stereoisomer indicated, unless otherwise noted. For example,

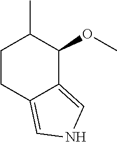

represents

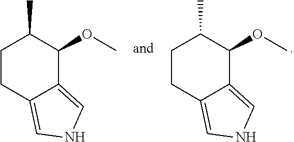

Similarly, for example, the chemical name (4R)-4-methoxy-5-methyl-4,5,6,7-tetrahydro-2H-isoindole represents (4R,5R)-4-methoxy-5-methyl-4,5,6,7-tetrahydro-2H-isoindole and (4R,5S)-4-methoxy-5-methyl-4,5,6,7-tetrahydro-2H-isoindole. A bond drawn with a wavy line may be used to indicate that both stereoisomers are encompassed. This is not to be confused with a wavy line drawn perpendicular to a bond which indicates the point of attachment of a group to the rest of the molecule.

The term “stereoisomer” or “stereoisomerically pure” compound refers to one stereoisomer (for example, geometric isomer, enantiomer, diastereomer and atropisomer) of a compound that is substantially free of other stereoisomers of that compound. For example, a stereoisomerically pure compound having one chiral center w ill be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by w eight of the other stereoisomers of the compound, greater than about 95% by weight of one stereoisomer of the compound and equal or less than about 5% by weight of the other stereoisomers of the compound, or greater than about 97% by weight of one stereoisomer of die compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. For example,

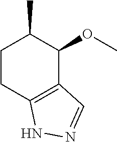

represents

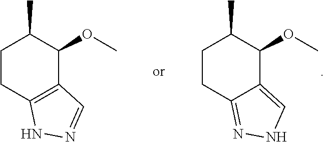

Similarly, for example, the chemical name (4R,5R)-4-methoxy-5-methyl-4,5,6,7-tetrahydro-1H-indazole represents (4R,5R)-4-methoxy-5-methyl-4,5,6,7-tetrahydro-1H-indazole and (4R,5R)-4-methoxy-5-methyl-4,5,6,7-tetrahydro-2H-indazole.

Accordingly, the scope of the instant disclosure is to be understood to encompass all tautomeric forms of the compounds disclosed herein.

### Isotopically-Labelled Compounds

Further, the scope of the present disclosure includes all pharmaceutically acceptable isotopically-labelled compounds of the compounds disclosed herein, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and 3H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S, Certain isotopically-labelled compounds of Formula I, for example, those incorporating a radioactive isotope, are useful in drug and/or substrate tissue distribution studies. The radioactive isotopes tritium (³H) and carbon-14 (¹⁴C are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. As such, the term “deuterated” refers to the substitution of one or more hydrogen atoms with one or more deuterium atoms on a particular structure or functional group. Substitution with positron emitting isotopes, such as ¹¹C, ¹⁸F, ¹⁵O and ¹³N, can be useful in Positron Emission Topography (PET) studies, for example, for examining target occupancy.

Isotopically-labelled compounds of the compounds disclosed herein can generally be prepared by conventional techniques known to those skilled in the art or by processes analogous to those described in the accompanying GENERAL SYNTHETIC PROCEDURES and EXAMPLES sections using an appropriate isotopically-labelled reagent in place of the non-labelled reagent previously employed.

### Definitions

This section will define additional terms used to describe the scope of the compounds, compositions and uses disclosed herein.

The following definitions are provided to assist in understanding the scope of this disclosure. Unless defined otherwise, all technical and scientific terms used herein have the same meaning as is commonly understood by one of ordinary skill in the art to which this disclosure belongs.

For purposes of this disclosure, the chemical elements are identified in accordance with the Periodic Table of the Elements, CAS version, Handbook of Chemistry and Physics, 75th Ed. Additionally, general principles of organic chemistry are described in Organic Chemistry, Thomas Sorrell, University Science Books, Sausalito: 1999, and March's Advanced Organic Chemistry, 5th Ed., Ed.: Smith, M. B, and March. J., John Wiley & Sons. New York: 2001, the entire contents of which are hereby incorporated by reference.

Unless otherwise indicated, the depictions of partial structures do not represent any particular orientation of the partial structure. For example, compounds of Formula (I) having

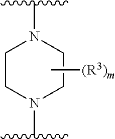

as

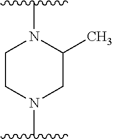 [SMILES: *N1CCN(*)C(C)C1]

includes compounds of Formula (I) depicted

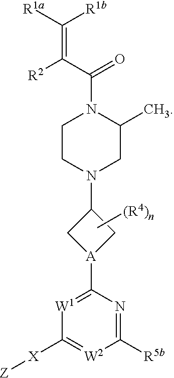

as or

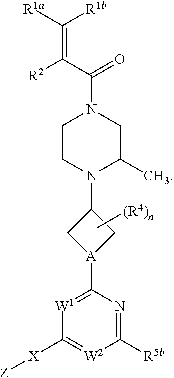

As described herein, compounds described herein may optionally be substituted with one or more substituents, such as illustrated generally below, or as exemplified by particular classes, subclasses, and species described herein. It will be appreciated that the phrase “optionally substituted” is used interchangeably with the phrase “substituted or unsubstituted.” In general, the term “substituted,” whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. Unless otherwise indicated, the substituent is selected from deuterium, halo, oxo, carboxyl, CHO, NH₂, amido, NO₂, ester, thioester, C₀₋₃ alkyleneCN, C₁₋₆ alkyl, C₁₋₆ haloalkyl, C₀₋₆ alkylene-OH, C₀₋₃ alkylene-C₁₋₄ alkoxy, C₀₋₃ alkylene-C₁₋₄ haloalkoxy, C₀₋₃ alkylene-C₁₋₄ thioalkoxy, C₀₋₆ alkylene-C₁₋₃ alkoxy, deuterated C₀₋₆ alkylene-OC₁₋₃ alkoxy, amido, C₀₋₂ alkylene-cycloalkyl having 3-7 total ring atoms, C₀₋₂ alkylene-cycloalkenyl having 5-7 total ring atoms, C₀₋₂ alkylene-heterocycloalkyl having 3-7 total ring atoms and 1-3 heteroatoms selected from N, O, and S, C₀₋₂ alkylene-heterocycloalkenyl having 3-7 total ring atoms and 1-3 heteroatoms selected from N, O, and S, and C₀₋₂ alkylene-C₆₋₁₀ aryl.

Selection of substituents and combinations of substituents contemplated herein are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some cases, a stable compound or chemically feasible compound is one that is not substantially altered when kept at a temperature of 40° C., or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

The term “halo” or “halogen” refers to fluoro (—F), chloro (—Cl), bromo (—Br), or iodo (—I).

The term “oxo” refers to ═O.

The term “ether” refers to an oxygen atom bonded to two alkyl or aryl groups (R—O—R). The term “ether bridge” refers to an ether group that forms a bridge on a ring, wherein the bridge has the indicated number of carbon atoms. For example, a C₁ ether bridge

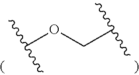 [SMILES: *CO*]

on a cyclohexylene ring cyclohexylene ring can be depicted as, for example,

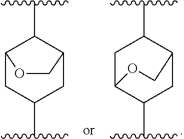 [SMILES: *C1CC2COC(C1)C2* ; *C1CC2OCC1CC2*]

The term “thioether” refers to a sulfur atom bonded to two alkyl or aryl groups (R—S—R). The term “thioether bridge” refers to a thioether group that forms a bridge on a ring, wherein the bridge has the indicated number of carbon atoms. For example, a C₁ thioether bridge

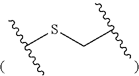 [SMILES: *CS*]

on a cyclohexylene ring cyclohexylene ring can be depicted as, for example,

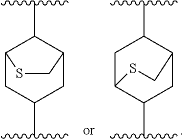 [SMILES: *C1CC2CSC(C1)C2* ; *C1CC2SCC1CC2*]

The term “alkyl” refers to a saturated straight or branched chain hydrocarbon containing the indicated number of carbon atoms. For example, C₃ alkyl means the alkyl group has 3 carbon atoms, C₁₋₆ alkyl refers to an alkyl group having a number of carbon atoms encompassing the entire range (e.g., 1, 2, 3, 4, 5, or 6 carbon atoms), as well as encompassing all subgroups (e.g., 1-2, 1-3, 1-4, 1-5, 1-6, 2-3, 2-4, 2-5, 2-6, 3-4, 3-5, 3-6, 4-5, 4-6, and 5-6 carbon atoms). Nonlimiting examples of alkyl groups include methyl, ethyl, n-propyl, isopropyl, n-butyl, sec-butyl, isobutyl, tert-butyl, pentyl, and hexyl.

The term “alkenyl” refers to a straight or branched chain hydrocarbon containing the indicated number of carbon atoms and one or more double bonds. For example, C₃ alkenyl means the alkenyl group has 3 carbon atoms, C₂₋₆ alkenyl refers to an alkenyl group having a number of carbon atoms encompassing the entire range (e.g., 2, 3, 4, 5, or 6 carbon atoms), as well as encompassing all subgroups (e.g., 2-3, 2-4, 2-5, 2-6, 3-4, 3-5, 3-6, 4-5, 4-6, and 5-6 carbon atoms). Nonlimiting examples of alkenyl groups include ethenyl, 1-propenyl, 2-propenyl, and butenyl.

The term “alkynyl” refers to a straight or branched chain hydrocarbon containing the indicated number of carbon atoms and one or more triple bonds. For example, C₃ alkynyl means the alkynyl group has 3 carbon atoms, C₂₋₆ alkynyl refers to an alkenyl group having a number of carbon atoms encompassing the entire range (e.g., 2, 3, 4, 5, and 6 carbon atoms), as well as encompassing all subgroups (e.g., 2-3, 2-4, 2-5, 2-6, 3-4, 3-5, 3-6, 4-5, 4-6, and 5-6 carbon atoms). Nonlimiting examples of alkynyl groups include ethynyl, 1-propynyl, 2-propynyl, and butynyl.

The term “alkylene” refers to a bivalent saturated aliphatic radical containing the indicated number of carbon atoms. For example, C₃ alkylene means the alkylene group has 3 carbon atoms, C₁₋₆ alkylene refers to an alkylene group having a number of carbon atoms encompassing the entire range (e.g., 1, 2, 3, 4, 5, or 6 carbon atoms), as well as encompassing all subgroups (e.g., 1-2, 1-3, 1-4, 1-5, 1-6, 2-3, 2-4, 2-5, 2-6, 3-4, 3-5, 3-6, 4-5, 4-6, and 5-6 carbon atoms). When the number of carbon atoms in an alkylene group is indicated as “C₀,” then the alkylene group is not present and the recited substituent is directly attached to the rest of the compound. For example, the term C₀₋₆ alkylene-OH indicates that the OH group can be directly attached to the compound or through a C₁₋₆ alkylene linker.

The term “alkylene bridge” refers to an alkylene group that forms a bridge on a ring, wherein the bridge has the indicated number of carbon atoms. For example, a C₃ alkylene bridge

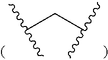 [SMILES: *C*]

on a cyclohexylene ring can be depicted as, for example,

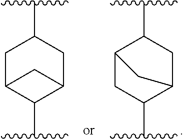 [SMILES: *C1CC2CC(C1)C2* ; *C1CC2CC1CC2*]

A C₂ alkylene bridge

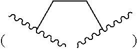 [SMILES: *CC*]

on a cyclohexylene ring can be depicted as, for example,

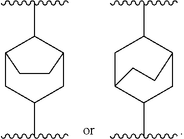 [SMILES: *C1CC2CCC(C1)C2* ; *C1CC2CCC1CC2*]

A C₃ alkylene bridge

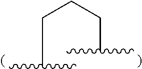 [SMILES: *CCC*]

on a cyclohexylene ring can be depicted as, for example

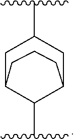 [SMILES: *C1CC2CCCC(C1)C2*]

Additional examples of rings having a C₁₋₂ alkylene bridge are

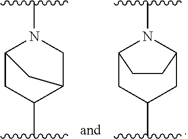 [SMILES: *C1CC2CC1CN2* ; *C1CC2CCC(C1)N2*]

The term “alkenylene” refers to a bivalent straight or branched chain hydrocarbon chain containing the indicated number of carbon atoms and one or more double bonds. For example, C₃ alkenylene means the alkenylene group has 3 carbon atoms, C₁₋₆ alkenylene refers to an alkenylene group having a number of carbon atoms encompassing the entire range (e.g., 1, 2, 3, 4, 5, or 6 carbon atoms), as well as encompassing all subgroups (e.g., 1-2, 1-3, 1-4, 1-5, 1-6, 2-3, 2-4, 2-5, 2-6, 3-4, 3-5, 3-6, 4-5, 4-6, and 5-6 carbon atoms).

The term “alkenylene bridge” refers to an alkenylene group that forms a bridge on a ring, wherein the bridge has the indicated number of carbon atoms. A C₂ alkenylene bridge

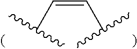 [SMILES: */C=C\*]

on a cyclohexylene ring can be depicted as, for example,

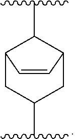 [SMILES: *C1CC2C=CC(C1)C2*]

A C₃ alkenylene bridge

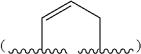 [SMILES: */C=C\C*]

on a cyclohexylene ring can be depicted as, for example,

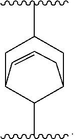 [SMILES: *C1CC2C=CCC(C1)C2*]

The term “heteroatom,” unless otherwise stated herein, refers to an atom that is not carbon or hydrogen. Examples of heteroatoms include oxygen, sulfur, nitrogen, or phosphorus.

The term “haloalkyl” refers to an alkyl group, as previously defined herein, in which one or more of the hydrogen atoms is replaced by a halogen. The halogen independently is selected at each occurrence. The term includes perfluorinated alkyl groups, such as CF₃ and CF₂CF₃. For example, the term “C₁₋₄ haloalkyl” refers to a C₁₋₄ alkyl as defined herein, wherein one or more hydrogen atoms are substituted with a halogen. Representative examples of C₁₋₄ haloalkyl include, but are not limited to, CH₂F, CHF₂, CF₃, CHFCl, CH₂CF₃, CFHCF₃, CF₂CF₃, CH(CF₃)₂, CF(CHF₂)₂, and CH(CH₂F)(CF₃).

The term “alkoxy” refers to an alkyl group, as previously defined herein, attached to the molecule through an oxygen atom (e.g., —O-alkyl). Nonlimiting examples of alkyl groups include methoxy, ethoxy, propoxy, iso-propoxy, and butoxy.

The term “thioalkoxy” refer to an alkyl group, as previously defined herein, attached to the molecule through a sulfur atom (e.g., —S-alkyl).

The term “haloalkoxy” refers to an alkoxyl group, as previously defined herein, in which one or more of the hydrogen atoms is replaced by a halogen. The term includes perfluorinated alkyl groups, such as OCF₃ and OCF₂CF₃. Representative examples of C₁₋₄ haloalkoxy include, but are not limited to, OCH₂F, OCHF₂, OCF₃, OCHFCl, OCH₂CF₃, OCFHCF₃, OCF₂CF₃, OCH(CF₃)₂, OCF(CHF₂)₂, and OCH(CH₂F)(CF₃).

The term “cycloalkyl” refers to an aliphatic cyclic hydrocarbon group containing the indicated number of carbon atoms in its ring. For example, C₅ cycloalkyl refers to a cycloalkyl group that has 5 carbon atoms in the ring, C₃₋₇ cycloalkyl refers to cycloalkyl group having a number of carbon atoms encompassing the entire range (e.g., 3, 4, 5, 6, and 7 carbon atoms in the ring), as well as encompassing all subgroups (e.g., 3-4, 3-5, 3-6, 3-7, 4-5, 4-6, 4-7, 5-6, 5-7, and 6-7 carbon atoms m the ring). Nonlimiting examples of cycloalkyl groups include cyclopropyl, cyclobutyl, cyclopentyl, and cyclohexyl. The term “spiro-cycloalkyl” refers to a cycloalkyl group as previously defined herein that is attached to the compound through one common atom. For example, a methylpiperidine ring that has a spiro-cyclopropyl group as a substituent can be depicted as:

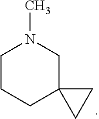

The terms “fused cycloalkyl ring” or “fused-cycloalkyl” can be used interchangeably and refer to a cycloalkyl group, as previously defined herein, that shares two adjacent atoms (i.e., one covalent bond) with the compound to which it is attached. For example, a methylpiperidine ring that has a fused cyclopropyl group as a substituent can be depicted as:

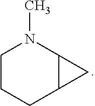

The term “cycloalkenyl” refers to a cyclic hydrocarbon group containing the indicated number of carbon atoms in its ring and one or more double bonds. For example, C₅ cycloalkenyl refers to a cycloalkenyl group that has 5 carbon atoms in the ring, C₅₋₇ cycloalkenyl refers to cycloalkenyl group having a number of carbon atoms encompassing the entire range (e.g., 5, 6, and 7 carbon atoms in the ring), as well as encompassing all subgroups (e.g., 5-6, 5-7, and 6-7 carbon atoms in the ring). Nonlimiting examples of cycloalkyl groups include cyclopentenyl, and cyclohexenyl.

The term “heterocycloalkyl” refers to a saturated ring comprising carbon and 1, 2, or 3 heteroatoms, and having the indicated number of total ring atoms (the sum of carbon atoms and heteroatoms in the ring). For example, a heterocycloalkyl having 5 total atoms and 2 heteroatoms selected from N and S, refers to a ring having 3 carbon atoms and 2 heteroatoms, wherein each heteroatom of the ring independently is N or S. As another example, a heterocycloalkyl having 5-7 total ring atoms and 1-3 heteroatoms selected from N, O, and S refers to a ring having a total number of ring atoms in the indicated range (e.g., 5, 6, or 7 total atoms), as well as encompassing all subgroups (e.g., 5-6 or 6-7 total ring atoms), wherein 1, 2, or 3 of the atoms in the ring are heteroatoms and each heteroatom independently is selected from N, O, and S. Thus, heterocycloalkyl having 5-7 total ring atoms and 1-3 heteroatoms selected from N, O, and S encompasses rings containing, for example, 4 carbon atoms and 1 heteroatom, 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing independently is selected from N, O, and S. Nonlimiting examples of heterocycloalkyl groups include but are not limited to aziridinyl, azetidinyl, oxetanyl, pyrrolidinyl, pyrazolidinyl, imidazolidinyl, oxazolidinyl, isoxazolediyl, thiazolidinyl, isothiazolidinyl, tetrahydrofuranyl, tetrahydrothiophenyl, piperidinyl, piperazinyl, tetrahydropyranyl, morpholinyl, azepanyl, diazepanyl, triazepanyl, oxazepanyl, azocanyl, diazepanyl, triazinanyl, oxazepanyl, thiazepanyl, and thiazepanyl. The term “spiro-heterocycloalkyl” refers to a heterocycloalkyl group as previously defined herein that is attached to the compound through one common atom. For example, a methylpiperidine ring that has a spiro-oxetanyl group as a substituent can be depicted as:

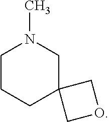

The term “fused-heterocycloalkyl” refers to a heterocycloalkyl group as previously defined herein that shares two adjacent atoms (i.e., one covalent bond) with the compound to which it is attached. For example, a methylpiperidine ring that has a fused-azetidinyl group as a substituent can be depicted as:

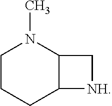

The term “heterocycloalkenyl” is defined similarly to “heterocycloalkyl” except that the ring contains one or more carbon-carbon double bonds.

The term “aryl” refers to an aromatic, carbocylic ring having the indicated number of carbon ring atoms. For example, Caryl refers to an aryl group that has 6 carbon atoms in the ring (e.g., phenyl). Aryl groups can be isolated (e.g., phenyl) or fused to another aryl group (e.g., naphthyl or anthracenyl).

The term “heteroaryl” refers to an aromatic ring comprising carbon and 1, 2, or 3 heteroatoms, and having the indicated number of total ring atoms (the sum of carbon atoms and heteroatoms in the ring). For example, a heteroaryl group having 5 total atoms and 2 heteroatoms selected from N and S, refers to an aromatic ring having 3 carbon atoms and 2 heteroatoms, wherein each heteroatom of the ring independently is N or S. As another example, a heteroaryl having 5-7 total ring atoms and 1-3 heteroatoms selected from N, O, and S refers to an aromatic ring having a total number of ring atoms in the indicated range (e.g., 5, 6, or 7 total atoms), as well as encompassing all subgroups (e.g., 5-6 or 6-7 total ring atoms), wherein 1, 2, or 3 of the atoms in the ring are heteroatoms and each heteroatom independently is selected from N, O, and S. Thus, heteroaryl having 5-7 total ring atoms and 1-3 heteroatoms selected from N, O, and S encompasses rings containing, for example, 4 carbon atoms and 1 heteroatom, 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing independently is selected from N, O, and S. Nonlimiting examples of heteroaryl groups include but are not limited to furanyl, imidazolyl, isothiazolyl, isoxazolyl, oxadiazolyl, oxazolyl, pyrazolyl, pyrrolyl, thiadiazolyl, thiazolyl, thiophenyl, tetrazolyl, triazinyl, triazolyl, pyridyl, pyridazinyl, pyrazinyl, pyrimidinyl, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyndinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl.

The term “bicyclic ring” refers a functional group that comprises two joined rings. Unless otherwise indicated, the bicyclic ring may be spirocyclic, in which the two rings share a single atom (e.g., a quaternary carbon atom), fused, in which the two rings share two adjacent atoms (i.e. one covalent bond), or bridged, in which to rings share three or more atoms and contain a bridge having at least one atom.

The terms “protecting group” and “protective group” as used herein, are interchangeable and refer to an agent used to temporarily block one or more desired functional groups in a compound with multiple reactive sites. In some cases, a protecting group has one or more, or specifically all, of the following characteristics: (a) is added selectively to a functional group in good yield to give a protected substrate that is (b) stable to reactions occurring at one or more of the other reactive sites; and (c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts. P G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. In some cases, nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

The term “bond” indicates that a specified functional group is absent.

The term “pharmaceutically acceptable” as used herein refers to a composition or a component of a composition that is generally safe, non-toxic, and neither biologically nor otherwise undesirable.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts, Properties, Selection, and Use. 2^{nd} Revised Edition (2011).

The terms “adjacent” refers to substituents that are attached to adjacent atoms along a chain or within a ring. Adjacent R groups along a chain and within a ring can be depicted as

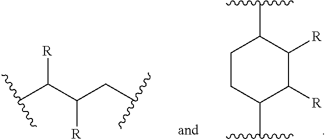

respectively.

The term “non-adjacent” refers to substituents that are attached to atoms along a chain or within a ring that are not attached to adjacent atoms and that are not geminal. Non-adjacent R groups along a chain and within a ring can be depicted as

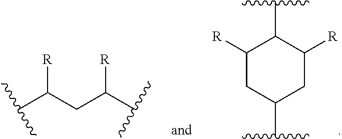

respectively.

The term “pharmaceutically acceptable” as used herein refers to a composition or a component of a composition that is generally safe, non-toxic, and neither biologically nor otherwise undesirable.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts, Properties, Selection, and Use, 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherents, glidants, disintegrants, flavoring agents, coatings, binders, sweeteners, lubricants, sorbents, preservatives, and the like.

The terms “subject” and “patient” as used herein are interchangeable and refer to humans and mammals, including, but not limited to, primates, cows, sheep, goats, horses, dogs, cats, rabbits, rats, and mice. In some cases, the subject is human.

The term “therapeutically effective amount” as used herein refers to that amount of a compound disclosed herein that will elicit the biological or medical response of a tissue, a system, or subject that is being sought by a researcher, veterinarian, medical doctor or other clinician.

The term “metastatic” refers to a cancer that has spread from the place where it first formed to another part of the body. The term non-metastatic refers to a cancer that has not spread from the place where it first formed to another part of the body.

The term “coupled exchange assay” or “2 h coupled exchange assay” as used herein refers to the assay described in the Section entitled “BIOLOGICAL EVALUATION.”

### GENERAL SYNTHETIC PROCEDURES

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following scheme. Variables used in the following scheme are the variables as defined for Formula I, unless otherwise noted. All starting materials are either commercially available, for example, from Merck Sigma-Aldrich Inc., Fluorochem Ltd., and Enamine Ltd, or known in the art and may be synthesized by employing known procedures using ordinary skill. Starting materials may also be synthesized via the procedures disclosed herein. Suitable reaction conditions, such as, solvent, reaction temperature, and reagents, for the Schemes discussed in this section, may be found in the examples provided herein. The abbreviation PG refers to a protecting group, as defined herein in the DEFINITIONS AND GENERAL TERMINOLOGY section. In the scheme below, each PG can be the same as or different from another PG in the compound, so long as each protecting group can be selectively removed.

In general, the compounds of Formula I can be synthesized according to Scheme 1, below.

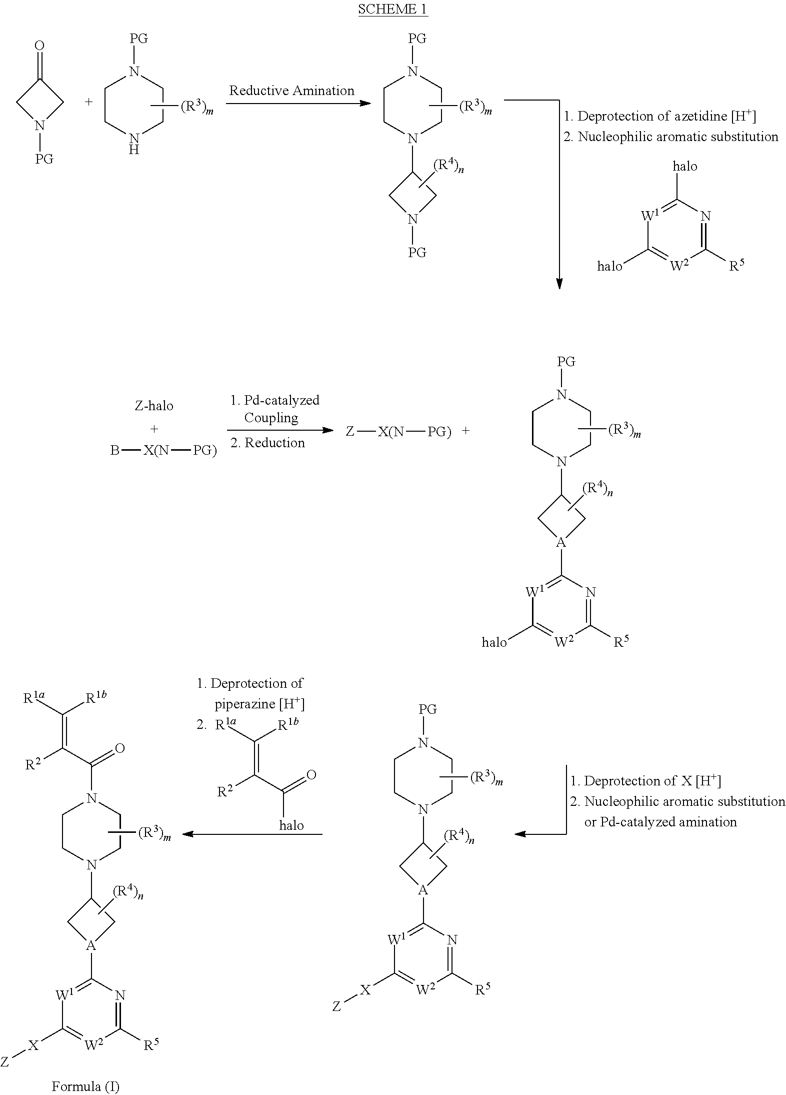

A nitrogen-protected linker portion of Formula (I):

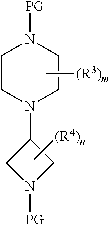

can be synthesized by reacting a desired nitrogen-protected 3-azetidinone with a desired nitrogen-protected piperazine in the presence of an appropriate reducing reagent, such as a borohydride reagent, in a reductive amination reaction. The resulting linker can be coupled to a desired, halogenated core, such as a pyrimidine core:

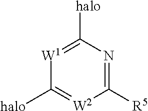

by deprotecting the nitrogen of the azetidine and then conducting a nucleophilic aromatic substitution reaction in the presence of an appropriate base to form the middle portion of the compound of Formula (I):

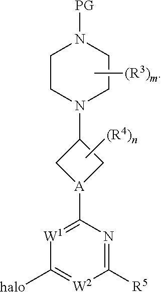

Variable Z can be synthesized by, for example, starting with a desired, optionally substituted, phenyl, heteroaryl, or bicyclic ring, and optionally attaching additional desired substituents to the ring through common techniques known to one skilled in the art. Z-halo can be prepared for coupling by halogenating the phenyl, heteroaryl, or bicyclic ring of Z using, for example, a suitable iodination reagent (e.g., N-iodosuccinimide), bromination reagent (e.g., CBr₄), or chlorination reagent (e.g., (CCl₃)₂) optionally in the presence of a suitable base. The tail portion of Formula (I) can be synthesized by reacting a desired halogenated variable Z (“Z-halo”) with a desired organoboron-functionalized variable X that comprises a protected nitrogen atom (“B—X(N-PG)”) in a palladium-catalyzed coupling reaction to form the Z—X(N-PG) tail portion of Formula (I) When Y of Formula (I) is other than N, then the double bond that results from the coupling reaction can optionally be reduced to a single bond.

The Z—X(N-PG) tail portion of Formula (I) can be coupled to the middle portion of Formula (I) by deprotecting the nitrogen atom of variable X in Z—X(N-PG) to form Z—X(NH), and then performing a nucleophilic aromatic substitution with the middle portion of Formula (I) and an appropriate base in a nucleophilic aromatic substitution reaction to form:

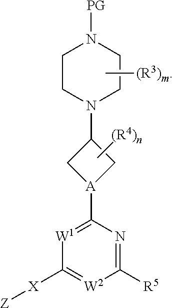

In some cases, the tail portion of Formula (I) can be installed via a palladium-catalyzed amination reaction, such as the Buchwald reaction. The Michael acceptor can be installed on the compound by deprotecting the nitrogen atom of the piperazine ring in the presence of an acid, such as TFA, and reacting the deprotected piperazine ring with a desired halogenated α,β-unsaturated ketone, such as acryloyl chloride to form the compound of Formula (I).

As easily recognized by a person of ordinary skill in the art, Scheme 1 can be adapted to synthesize compounds of Formula (I) in which W¹ is not N by using an appropriate core starting material.

Accordingly, a further aspect of the disclosure provides a process for preparing compounds of Formula (I) (e.g., (e.g., a compound of Formula (I), a compound of Formula (I′), a compound of Formula (IA), a compound of Formula (IB), a compound of Formula (IC), a compound of Formula (ID), a compound of Formula (IE), a compound of Formula (IF), a compound listed in Table A, a compound listed in Table B, a compound listed in Table C, a compound listed in Table D, or a compound listed in Table E), or a pharmaceutically acceptable salt of any of the foregoing, comprising converting an intermediate described herein to a compound of Formula (I).

As can be appreciated by the skilled artisan, the above synthetic scheme and representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid, gas phase, and supercritical fluid), extraction, distillation, trituration, and reverse phase HPLC.

The disclosure further encompasses “intermediate” compounds, including structures produced from the synthetic procedures described, whether isolated or generated in-situ and not isolated, prior to obtaining the finally desired compound. These intermediates are included in the scope of this disclosure. In some cases, the disclosure provides an intermediate selected from Intermediate A-1 to A-53, or pharmaceutically acceptable salts of any of the foregoing, the structures of which can be found in the EXAMPLES section, below. In some cases, the disclosure provides an intermediate selected from Intermediate B-1 to B-110, or pharmaceutically acceptable salts of any of the foregoing, the structures of which can be found in the EXAMPLES section, below. In some cases, the disclosure provides an intermediate selected from Intermediate C-1 to C-17, or pharmaceutically acceptable salts of any of the foregoing, the structures of which can be found in the EXAMPLES section, below. In some cases, the disclosure provides an intermediate selected from Intermediate D-1 to D-15, or pharmaceutically acceptable salts of any of the foregoing, the structures of which can be found in the EXAMPLES section, below. In some cases, the disclosure provides an intermediate selected from Intermediate E-1 to E-20, or pharmaceutically acceptable salts of any of the foregoing, the structures of which can be found in the EXAMPLES section, below. In some cases, the disclosure provides an intermediate selected from Intermediate F-1 to F-7, or pharmaceutically acceptable salts of any of the foregoing, the structures of which can be found in the EXAMPLES section, below. In some cases, the disclosure provides an intermediate selected from Intermediate G-1 to G-2, or pharmaceutically acceptable salts of any of the foregoing, the structures of which can be found in the EXAMPLES section, below. In some cases, the disclosure provides an intermediate selected from Intermediate H-1-1, H-2-2, H-3-1, H-3-2, H-4-1, H-4-2, H-5-1, H-5-2, H-6-1, H-6-2, H-7-1, H-7-2, H-8-1, H-8-2, H-9-1, H-9-2, H-10-1, H-10-2, H-11-1, H-11-2, H-12-1, H-13-1, H-14-1, H-15-1, H-16-1, H-16-2, H-17-1, H-17-2, H-17-3, H-18-1, H-18-2, H-19-1, H-19-2, H-20-1, H-21-1, 11-21-2, 11-22-1, 11-22-2, H-23-3, 11-24-2, 11-254, 11-26-1, H-26-2, H-27-3, 11-28-1, H-29-1, H-29-2, H-30-1, H-31-1, H-31-2, H-32-2, H-33-1, H-33-2, H-34-1, H-34-2, H-35-1, H- 35-2, H-36-1, H-36-2, 11-37-1, H-37-2, H-38-1, H-39-1, 11-40-1, H-40-2, H-41-1, H-42-1, 11-43-1, H-43-2, H-44-1, H-45-1, H46-1, H-46-2, and H-47-1, or pharmaceutically acceptable salts of any of the foregoing, the structures of which can be found in the EXAMPLES section, below.

### Other Embodiments

Provided herein as Embodiment 1 is a compound of Formula (I):

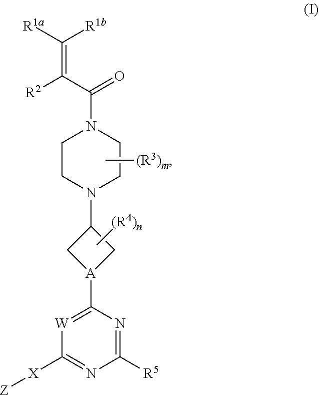

or a pharmaceutically acceptable salt thereof,
wherein:
m is 0, 1, 2, 3, or 4;
n is 0, 1, or 2;
is 0, 1, 2, 3, or 4;
A is N, CH, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy;
W is CH, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy;
X is

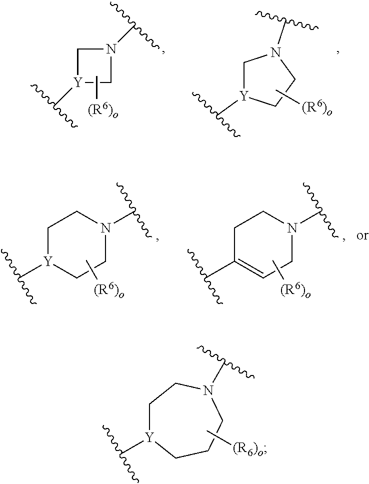

Y is N, C—H, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein each of the phenyl, heteroaryl, and bicyclic rings is optionally substituted with 1-4 substituents;
each of R^{1a}, R^{1b}, and R² independently is H, D, halo, C₁₋₄ alkyl, C₁₋₂ haloalkyl, C₁₋₂ alkylene-OH, C₀₋₂ alkylene-C₃₋₄ alkoxy, C₀₋₂ alkylene-C₁₋₄ haloalkoxy, C₀₋₂ alkylene-CN, C₀₋₂ alkylene-N(R^{N1})₂, C₁₋₂ alkylene-heterocycloalkyl having 3-6 total ring atoms and 1 or 2 heteroatoms selected from N, O, and S, or R^{1b} and R², together with the carbon atoms to which they are attached, from a

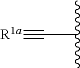

group;
each R³ independently is C₁₋₃ alkyl, C₁₋₃ haloalkyl, C₀₋₃ alkyleneCN, C₀₋₃ alkyleneOH, C₀₋₃ alkylene-C₁₋₃ alkoxy, oxo, spiro-cycloalkyl having 3-7 total ring atoms, spiro-heterocycloalkyl having 3-7 total ring atoms and 1 or 2 heteroatoms selected from N, O and S, or two adjacent R—, together with the atoms to which they are attached, form a fused cycloalkyl ring having 3-7 total ring atoms;
each R⁴ independently is C₁₋₃ alkyl, C₁₋₃ haloalkyl, C₀₋₃ alkyleneCN, C₁₋₃ alkyleneOH, C₁₋₃ alkylene-C₁₋₃ alkoxy, oxo, spiro-cycloalkyl having 3-7 total ring atoms, or spiro-heterocycloalkyl having 4-7 total ring atoms and 1 or 2 heteroatoms selected from N, O and S;
R⁵ is C₁₋₃ haloalkyl, C₁₋₄ alkyl, C₂₋₃ alkenyl, C₂₋₃ alkynyl, halo, C₁₋₃ alkoxy, C₁₋₃ thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing is independently optionally substituted with 1-3 substituents;
each R⁶ independently is halo, CN, oxo, C₁₋₃ alky C₁₋₃ haloalkyl, C₀₋₃ alkyleneOH, C₀₋₃ alkylene-C₁₋₃ alkoxy, deuterated C₀₋₃ alkylene-C₁₋₃ alkoxy, C₁₋₄ alkylene-N(R^{N1})₂, spiro-cycloalkyl having 3-7 total ring atoms, spiro-heterocycloalkyl having 4-7 total ring atoms and 1 or 2 heteroatoms selected from N, O and S, or
   two adjacent R⁶, together with the atoms to which they are attached, form a fused cycloalkyl ring having 3-7 total ring atoms; or
   Y and an adjacent R⁶, together with the atoms to which they are attached, form a fused cycloalkyl ring having 3-7 total ring atoms; wherein the fused cycloalkyl ring of any of the foregoing is optionally substituted with 1 or 2 substituents; or
   two non-adjacent R⁶ join together to form a C₁₋₃ alkylene bridge or a C₁₋₃ ether bridge; and
each R⁶ independently is H or C₁₋₄ alkyl.

Provided herein as Embodiment 2 is the compound or salt of Embodiment 1, wherein at least one of R^{1a}, R^{1b}, and R² is H or D.

Provided herein as Embodiment 3 is the compound or salt of Embodiment 2, wherein each of R^{1a}, R^{1b}, and R² independently is H or D.

Provided herein as Embodiment 4 is the compound or salt of Embodiment 3, wherein each of R^{1a}, R^{1b}, and R² independently is H.

Provided herein as Embodiment 5 is the compound or salt of Embodiment 3, wherein each of R^{1a}, R^{1b}, and R² independently is D.

Provided herein as Embodiment 6 is the compound or salt of Embodiment 1 or 2, wherein at least one of R^{1a}, R^{1b}, and R² is halo.

Provided herein as Embodiment 7 is the compound or salt of Embodiment 6, wherein R^{1a} is halo and each of R^{1b} and R is H.

Provided herein as Embodiment 8 is the compound or salt of Embodiment 6 or 7, wherein each halo independently is Br, Cl, or F.

Provided herein as Embodiment 9 is the compound or salt of Embodiment 1 or 2, wherein at least one of R^{1a}, R^{1b}, and R² is C₁₋₄ alkyl or C₁₋₄ haloalkyl.

Provided herein as Embodiment 10 is the compound or salt of Embodiment 9, wherein at least one of R^{1a}, R^{1b}, and R² is CH₃ or CF₁.

Provided herein as Embodiment 11 is the compound or salt of Embodiment 1 or 2. H herein at least one of R^{1a}, R^{1b}, and R² is C₁₋₂ alkylene-OH, C₀₋₂ alkylene-C₁₋₄ alkoxy, C₀₋₂ alkylene-C₁₋₄ haloalkoxy, C₀₋₂ alkylene-CN, or C₀₋₂ alkylene-N(R^{N1})₂.

Provided herein as Embodiment 12 is the compound or salt of Embodiment 11, wherein each R^{N1} independently is H or CH₃.

Provided herein as Embodiment 13 is the compound or salt of Embodiment 12, wherein each R^{N1} independently is H.

Provided herein as Embodiment 14 is the compound or salt of Embodiment 11 or 12, wherein at least one of R^{1a}, R^{1b}, and R² is CH₂OH, OCH₃, CH₂OC H, OCF₃, CH₂OCF₃, CN, CH₂CN, NH₂, N(CH)₂, CH₂NH₂, or CH₂N(CH₃)₂.

Provided herein as Embodiment 15 is the compound or salt of Embodiment 1 or 2, wherein at least one of R^{1a}, R^{1b}, and R² is C₁₋₂ alkylene-heterocycloalkyl having 3-6 total ring atoms and 1 or 2 heteroatoms selected from N, O, and S.

Provided herein as Embodiment 16 is the compound or salt of Embodiment 15, wherein the heterocycloalkyl is aziridinyl, azetidinyl, pyrrolidinyl, piperidinyl, or morpholinyl.

Provided herein as Embodiment 17 is the compound or salt of Embodiment 16, wherein at least one of R^{1a}, R^{1b}, and R² is aziridin-1-yl-methyl, azetidin-1-yl-methyl, pyrrolidine-1-yl-methyl, piperidin-1-yl-methyl, morpholin-1-yl-methyl.

Provided herein as Embodiment 18 is the compound or salt of Embodiment 1, wherein R^{1b} and R², together with the carbon atoms to winch they are attached, from a

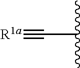

group.

Provided herein as Embodiment 19 is the compound or salt of Embodiment 1, wherein

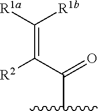

is

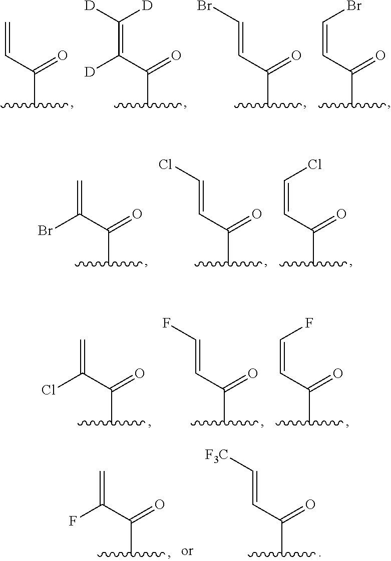

Provided herein as Embodiment 20 is the compound or salt of Embodiment 19, wherein

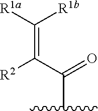

is

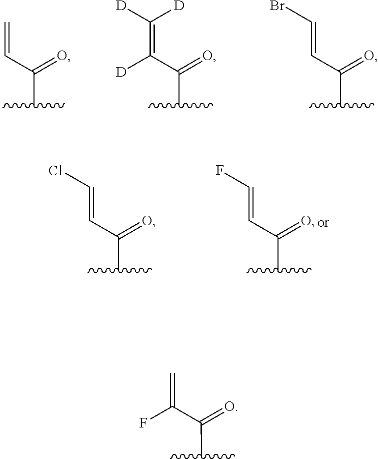

Provided herein as Embodiment 21 is the compound or salt of any one of Embodiments 1-20, wherein m is 0.

Provided herein as Embodiment 22 is the compound or salt of any one of Embodiments 1-20, wherein m is 1.

Provided herein as Embodiment 23 is the compound or salt of any one of Embodiments 1-20, wherein m is 2.

Provided herein as Embodiment 24 is the compound or salt of any one of Embodiments 1-20, wherein m is 3.

Provided herein as Embodiment 25 is the compound or salt of any one of Embodiments 1-20, wherein m is 4.

Provided herein as Embodiment 26 is the compound of salt of any one of Embodiments 22-25, wherein at least one R³ is C₁₋₃ alkyl or C₁₋₃ haloalkyl.

Provided herein as Embodiment 27 is the compound or salt of Embodiment 26, wherein at least one R³ is CH₃, CH₂CH₃, CF₃, CHF₂, or CH₂F.

Provided herein as Embodiment 28 is the compound or salt of any one of Embodiments 22-25, wherein at least one R³ is C₀₋₃ alkyleneCN.

Provided herein as Embodiment 29 is the compound or salt of Embodiment 28, wherein at least one R³ is CN or CH₂CN.

Provided herein as Embodiment 30 is the compound or salt of any one of Embodiments 22-25, wherein at least one R³ is C₀₋₃ alkyleneOH or C₁₋₃ alkylene-C₁₋₃ alkoxy.

Provided herein as Embodiment 31 is the compound or salt of Embodiment 30, wherein at least one R³ is OH, CH₂OH, CH₂CH₂OH, OCH₁, CH₂OCH₃, or CH₂CH₂OCH₃.

Provided herein as Embodiment 32 is the compound or salt of any one of Embodiments 22-25, wherein at least one R³ is oxo.

Provided herein as Embodiment 33 is the compound or salt of any one of Embodiments 22-25, wherein at least one R³ is spiro-cycloalkyl having 3-7 total ring atoms or spiro-heterocycloalkyl having 3-7 total ring atoms and 1 or 2 heteroatoms selected from N, O and S.

Provided herein as Embodiment 34 is the compound or salt of Embodiment 33, wherein at least one R³ is spiro-cyclopropyl, spiro-cyclobutyl, spiro-oxetanyl, or spiro-tetrahydrofuranyl.

Provided herein as Embodiment 35 is the compound or salt of any one of Embodiments 22-25, wherein two adjacent R³, together with the atoms to which they are attached, form a fused cycloalkyl ring having 3-7 total ring atoms.

Provided herein as Embodiment 36 is the compound or salt of Embodiment 35, wherein two adjacent R¹, together with the atoms to which they are attached, form a fused cyclopropyl ring or a fused cyclobutyl ring.

Provided herein as Embodiment 37 is the compound or salt of any one of Embodiments 22-25, wherein each R³ independently is CH₃, CH₂CH₃, CF₃, CHF₂, CH₂F, CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl.

Provided herein as Embodiment 38 is the compound or salt of an % one of Embodiments 1-20, wherein m is 0, or m is 1 and R³ is CH₃, CF₃, CHF₂, CH₂F, CN, CH₂CN, CH₂OH, CH₂OCH₃, or spiro-oxetanyl.

Provided herein as Embodiment 39 is the compound or salt of any one of Embodiments 1-20, wherein

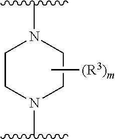

is

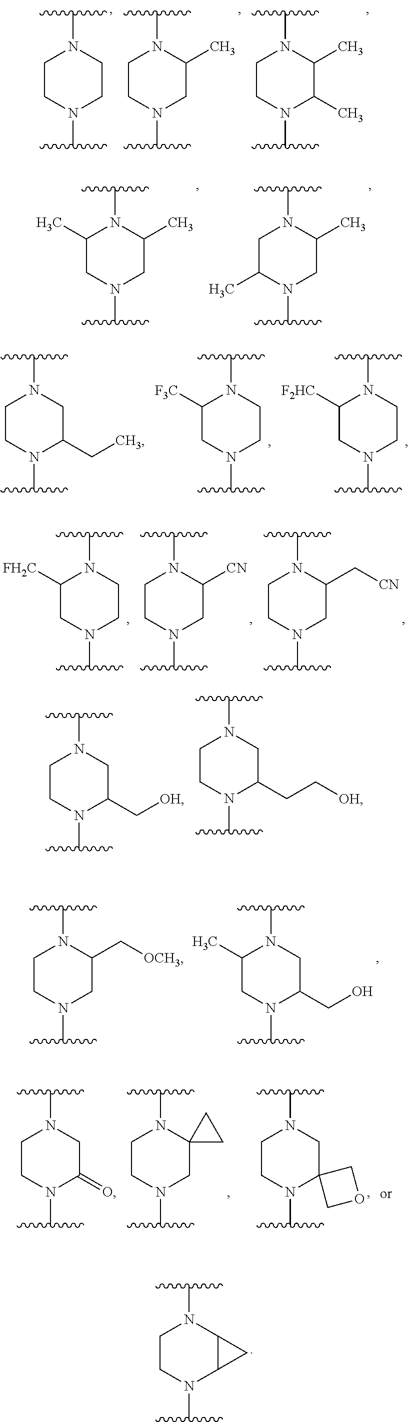

Provided herein as Embodiment 40 is the compound or salt of Embodiment 39, wherein

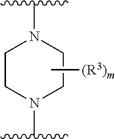

is

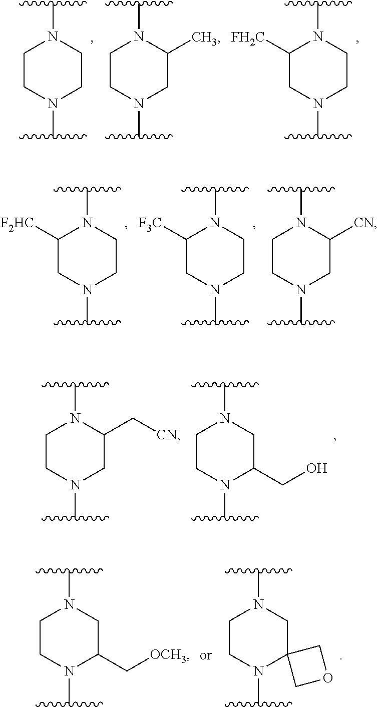

Provided herein as Embodiment 41 is the compound or salt of any one of Embodiments 1-40, wherein A is N.

Provided herein as Embodiment 42 is the compound or salt of any one of Embodiments 140, wherein A is CH, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy.

Provided herein as Embodiment 43 is the compound or salt of Embodiment 42, wherein A is CH.

Provided herein as Embodiment 44 is the compound or salt of Embodiment 42, wherein A is C—F, C—Cl, or C—CN.

Provided herein as Embodiment 45 is the compound or salt of Embodiment 42, wherein A is C—C₁₋₃ alkyl or C—C₁₋₃ haloalkyl.

Provided herein as Embodiment 46 is the compound or salt of Embodiment 45, wherein. A is C—CH₃, C—CH₂F, C—CHF₂, or C—CF₃.

Provided herein as Embodiment 47 is the compound or salt of Embodiment 42, wherein A is C—C₀₋₃ alkyleneOH or C—C₀₋₃ alkylene-C₁₋₄ alkoxy.

Provided herein as Embodiment 48 is the compound or salt of Embodiment 47, wherein A is C—OH, C—CH₂OH, C—OCH₃, or C—CH₂OCH₃.

Provided herein as Embodiment 49 is the compound or salt of any one of Embodiments 1-48, wherein n is 0.

Provided herein as Embodiment 50 is the compound or salt of any one of Embodiments 1-48, wherein n is 1.

Provided herein as Embodiment 51 is the compound or salt of any one of Embodiments 1-48, wherein n is 2.

Provided herein as Embodiment 52 is the compound or salt of Embodiment 50 or 51, wherein at least one R⁴ is C₁₋₃ alkyl or C₁₋₃ haloalkyl.

Provided herein as Embodiment 53 is the compound or salt of Embodiment 52, wherein at least one R⁴ is CH₃, CH₂CH₃, CH₂CH₂CH₃, CH(CH₃)₂, CF₃, CHF₂, or CH₂F.

Provided herein as Embodiment 54 is the compound or salt of Embodiment 50 or 51, wherein at least one R⁴ is C₀₋₃ alkyleneCN.

Provided herein as Embodiment 55 is the compound or salt of Embodiment 54, wherein at least one R⁴ is CN or CH₂CN.

Provided herein as Embodiment 56 is the compound or salt of Embodiment 50 or 51, wherein at least one R⁴ is C₁₋₃ alkyleneOH or C₁₋₃ alkylene-C₁₋₃ alkoxy.

Provided herein as Embodiment 57 is the compound or salt of Embodiment 56, wherein at least one R⁴ is CH₂OH, CH₂CH₂OH, CH₂OCH₃, or CH₂CH₂OCH₃.

Provided herein as Embodiment 58 is the compound or salt Embodiment 50 or 51, wherein at least one R³ is oxo.

Provided herein as Embodiment 59 is the compound or salt of Embodiment 50 or 51, wherein at least one R³ is spiro-cycloalkyl having 3-7 total ring atoms or spiro-heterocycloalkyl having 4-7 total ring atoms and 1 or 2 heteroatoms selected from N, O and S.

Provided herein as Embodiment 60 is the compound or salt of Embodiment 59, wherein at least one R⁴ is spiro-cyclopropyl, spiro-cyclobutyl, or spiro-oxetanyl.

Provided herein as Embodiment 61 is the compound or salt of Embodiment 41, wherein

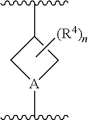

is

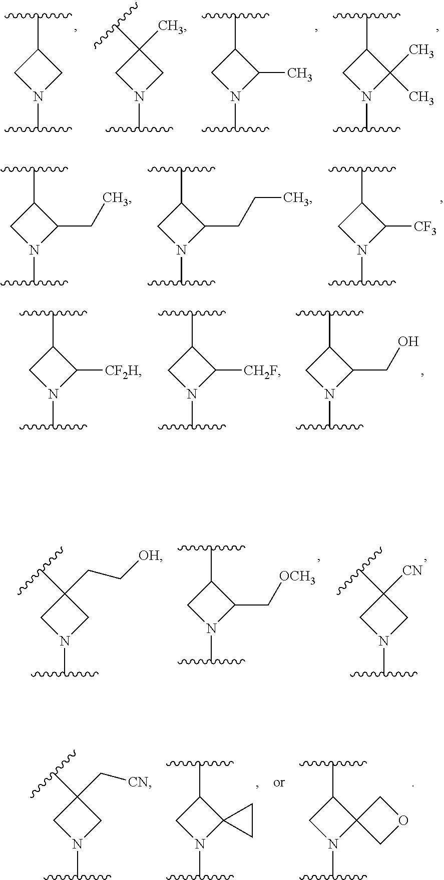

Provided herein as Embodiment 62 is the compound or salt of Embodiment 61, wherein

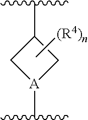

is

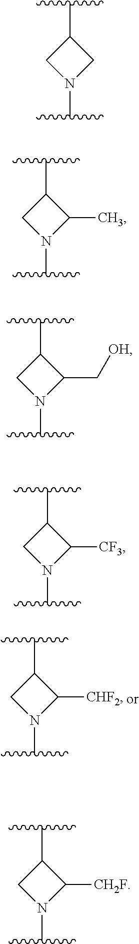

Provided herein as Embodiment 63 is the compound or salt of Embodiment 62, wherein

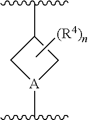

is

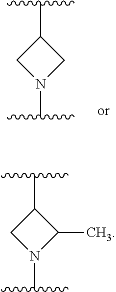 [SMILES: *C1CN(*)C1 ; *C1CN(*)C1C]

Provided herein as Embodiment 64 is the compound or salt of Embodiment 42, wherein

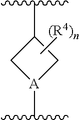

is

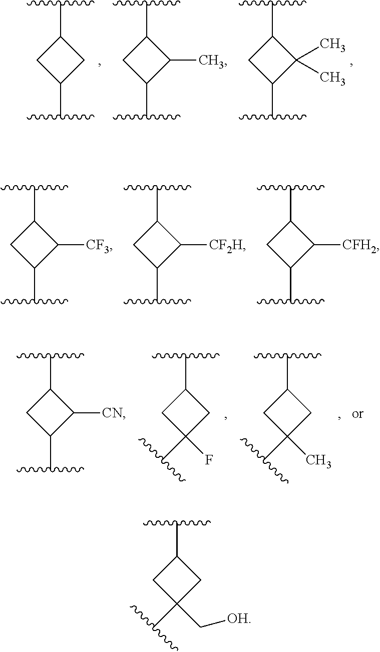

Provided herein as Embodiment 65 is the compound or salt of any one of Embodiments 1-64, wherein W is CH.

Provided herein as Embodiment 66 is the compound or salt of any one of Embodiments 1-64, wherein W is C—F, C—Cl, or C—CN.

Provided herein as Embodiment 67 is the compound or salt of any one of Embodiments 1-64, wherein W is C—C₁₋₃ alkyl or C—C₁₋₃ haloalkyl.

Provided herein as Embodiment 68 is the compound or salt of Embodiment 67, wherein W is C—CH; or C—CH₂CH₃.

Provided herein as Embodiment 69 is the compound or salt of any one of Embodiments 1-64, wherein W is C—C₀₋₃ alkyleneOH or C—C₀₋₃ alkylene-C₁₋₄ alkoxy.

Provided herein as Embodiment 70 is the compound or salt of Embodiment 69, wherein, W is C—OH, C—CH₂OH, C—OCH₃, or C—CH₂OCH₃.

Provided herein as Embodiment 71 is the compound or salt of any one of Embodiments 1-70, wherein R⁵ is C₁₋₃ haloalkyl.

Provided herein as Embodiment 72 is the compound or salt of Embodiment 71, wherein R⁵ is CF₃, CF₂H, CFH₂, or CF₂CH₃.

Provided herein as Embodiment 73 is the compound or salt of any one of Embodiments 1-70, wherein R⁵ is Br, Cl, or F.

Provided herein as Embodiment 74 is the compound or salt of any one of Embodiments 1-70, wherein R⁵ is C₁₋₃ alkoxy or C₁₋₃ thioalkoxy.

Provided herein as Embodiment 75 is the compound or salt of Embodiment 74, wherein R³ is OCH₃, or SCH₃.

Provided herein as Embodiment 76 is the compound or salt of any one of Embodiments 1-70, wherein R⁵ is C₁₋₄ alkyl, C₂₋₃ alkenyl, or C₂₋₃ alkynyl, optionally wherein each of the alkyl, alkenyl, and alkynyl is independently substituted with 1, 2, or 3 substituents selected from C₁₋₃ alkyl, C₁₋₃ haloalkyl, C₀₋₆ alkylene(OH), C₀₋₆ alkylene-C₁₋₃ alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 3-7 total ring atoms and 1-3 heteroatoms selected from N, O, and S, and phenyl.

Provided herein as Embodiment 77 is the compound or salt of Embodiment 76, wherein each of the 1, 2, or 3 substituents independently is selected from CH₃, CF₃, CF₂H, CFH₂, OH, OCH₃, OCF₃, CH₂OH, CH₂OCH₃, cyclopropyl, cyclobutyl, and phenyl.

Provided herein as Embodiment 78 is the compound or salt of Embodiment 77, wherein R⁵ is CH₃, CH₂CH₃, CH₂CH₂CH₃, CH(CH₃)₂,

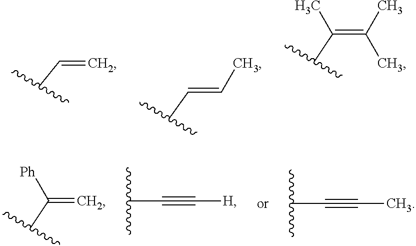

Provided herein as Embodiment 79 is the compound or salt of any one of Embodiments 1-64, wherein W is CH, and R is CF₃, CF₂H, or CFH₂.

Provided herein as Embodiment 80 is the compound or salt of any one of Embodiments 1-64, wherein

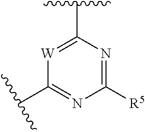

is

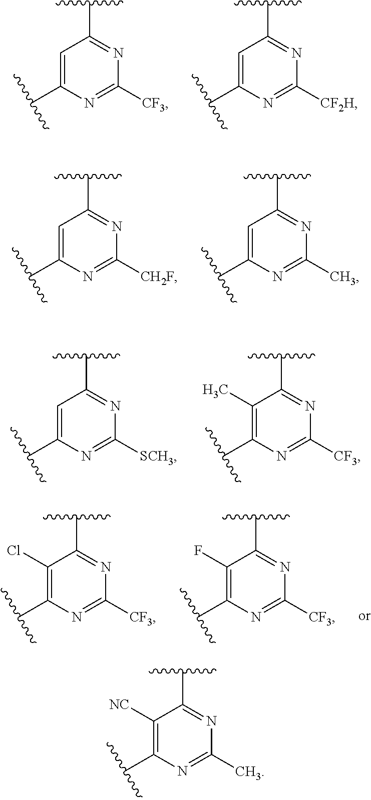

Provided herein as Embodiment 81 is the compound or salt of Embodiment 80, wherein

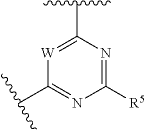

is

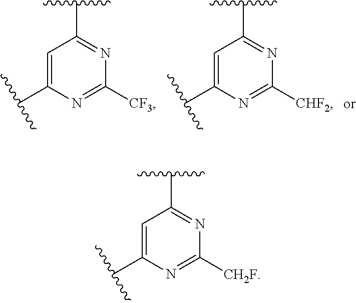

Provided herein as Embodiment 82 is the compound or salt of any one of Embodiments 1-81, wherein X is

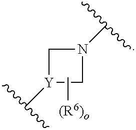

Provided herein as Embodiment 83 is the compound or salt of any one of Embodiments 1-81, wherein X is

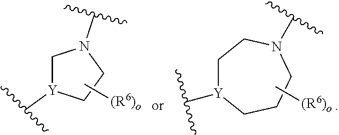

Provided herein as Embodiment 84 is the compound or salt of any one of Embodiments 1-81, wherein X is

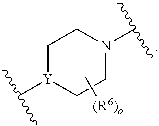

Provided herein as Embodiment 85 is the compound or salt of any one of Embodiments 1-81, wherein X is

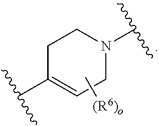

Provided herein as Embodiment 86 is the compound or salt of any one of Embodiments 1-84, wherein Y is N.

Provided herein as Embodiment 87 is the compound or salt of any one of Embodiments 1-84, wherein Y is C—H.

Provided herein as Embodiment 88 is the compound or salt of any one of Embodiments 1-84, wherein Y is C-halo, C—CN, C—C₁₋₃ alkyl, C—C₁₋₃ haloalkyl, C—C₀₋₃ alkyleneOH, or C—C₀₋₃ alkylene-C₁₋₄ alkoxy.

Provided herein as Embodiment 89 is the compound or salt of Embodiment 88, wherein Y is C—F, C—Cl, C—CH₃, C—CH₂CH₃, C—CH₂F, C—CHF₂, C—CF₃, C—OH, C—CH₂OH, C—OCH₃, or C—CH₂OCH₃.

Provided herein as Embodiment 90 is the compound or salt of any one of Embodiments 1-89, wherein o is 0.

Provided herein as Embodiment 91 is the compound or salt of any one of Embodiments 1-89, wherein o is 1.

Provided herein as Embodiment 92 is the compound or salt of any one of Embodiments 1-89, wherein o is 2.

Provided herein as Embodiment 93 is the compound or salt of any one of Embodiments 1-89, wherein o is 3.

Provided herein as Embodiment 94 is the compound or salt of any one of Embodiments 1-89, wherein o is 4.

Provided herein as Embodiment 95 is the compound or salt of any one of Embodiments 91-94, wherein at least one R⁶ is Br, Cl, F, CN, or oxo.

Provided herein as Embodiment 96 is the compound or salt of Embodiment 95, wherein at least one R³ is F.

Provided herein as Embodiment 97 is the compound or salt of any one of Embodiments 91-94, wherein at least one R⁶ is C₁₋₃ alkyl or C₁₋₃ haloalkyl.

Provided herein as Embodiment 98 is the compound or salt of Embodiment 97, wherein at least one R⁶ is CH₃, CH₂F, CHF₂, or CF₃.

Provided herein as Embodiment 99 is the compound or salt of any one of Embodiments 91-94, wherein at least one R⁶ is C₀₋₃ alkyleneOH, C₀₋₃ alkylene-C₁₋₃ alkoxy, deuterated C₀₋₃ alkylene-C₁₋₃ alkoxy, or C₁₋₄ alkylene-N(R^{N1})₂, and each R^{N1} independently is H or CH₃.

Provided herein as Embodiment 100 is the compound or salt of Embodiment 99, wherein at least one R⁶ is OH, CH₂OH, OCH₃, OCD₃, or CH₂OCH₃.

Provided herein as Embodiment 101 is the compound or salt of any one of Embodiments 91-94, wherein at least one R⁴ is spiro-cycloalkyl having 3-7 total ring atoms or spiro-heterocycloalkyl having 4-7 total ring atoms and 1 or 2 heteroatoms selected from N, O and S.

Provided herein as Embodiment 102 is the compound or salt of Embodiment 101, wherein at least one R⁶ is spiro-cyclopropyl, spiro-cyclobutyl, spiro-oxetanyl, or spiro-tetrahydrofuranyl.

Provided herein as Embodiment 103 is the compound or salt of Embodiment 102, wherein R³ is spiro-cyclopropyl.

Provided herein as Embodiment 104 is the compound or salt of any one of Embodiments 91-94, wherein two adjacent R⁶, together with the atoms to which they are attached, form a fused cycloalkyl ring having 3-7 total ring atoms; or Y and an adjacent R⁶, together with the atoms to which they are attached, form a fused cycloalkyl ring having 3-7 total ring atoms; wherein the fused cycloalkyl ring of any of the foregoing is optionally substituted with 1 or 2 substituents selected from halo, OH, C₁₋₃ alkoxy, or CN.

Provided herein as Embodiment 105 is the compound or salt of Embodiment 104, wherein the fused cycloalkyl ring is fused-cyclopropyl, fused-cyclobutyl, or fused-cyclopentyl.

Provided herein as Embodiment 106 is the compound or salt of any one of Embodiments 91-94, wherein two non-adjacent R⁶ join together to form a C₁₋₃ alkylene bridge or a C₁₋₃ ether bridge.

Provided herein as Embodiment 107 is the compound or salt of any one of Embodiments 1-82, wherein X is

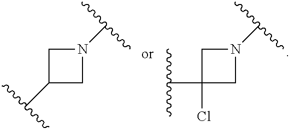 [SMILES: *C1CN(*)C1 ; *N1CC(*)(Cl)C1]

Provided herein as Embodiment 108 is the compound or salt of any one of Embodiments 1-81 and 83, wherein X is

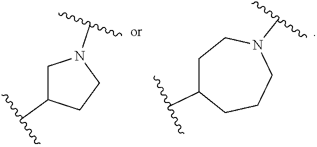 [SMILES: *C1CCN(*)C1 ; *C1CCCN(*)CC1]

Provided herein as Embodiment 109 is the compound or salt of any one of Embodiments 1-81 and 84, wherein X is

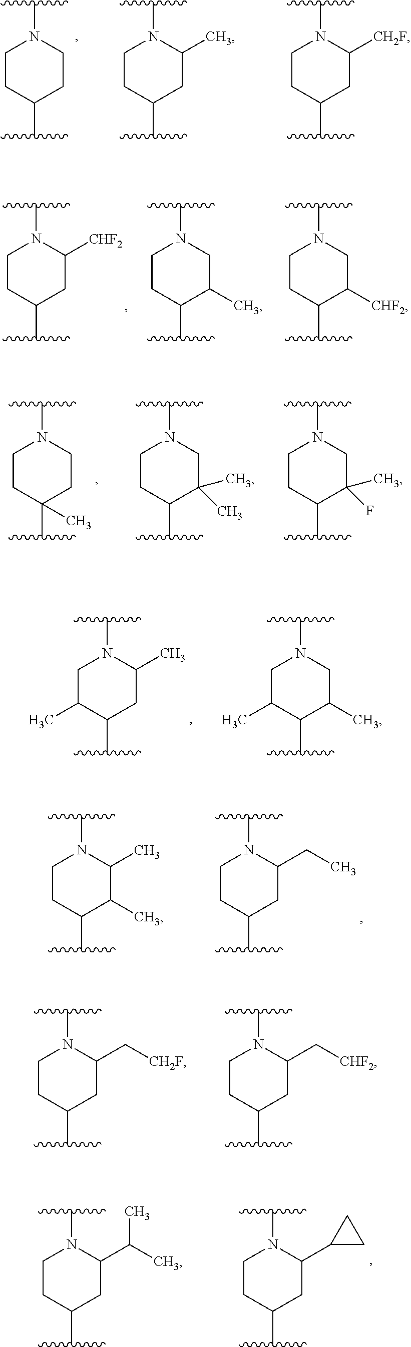

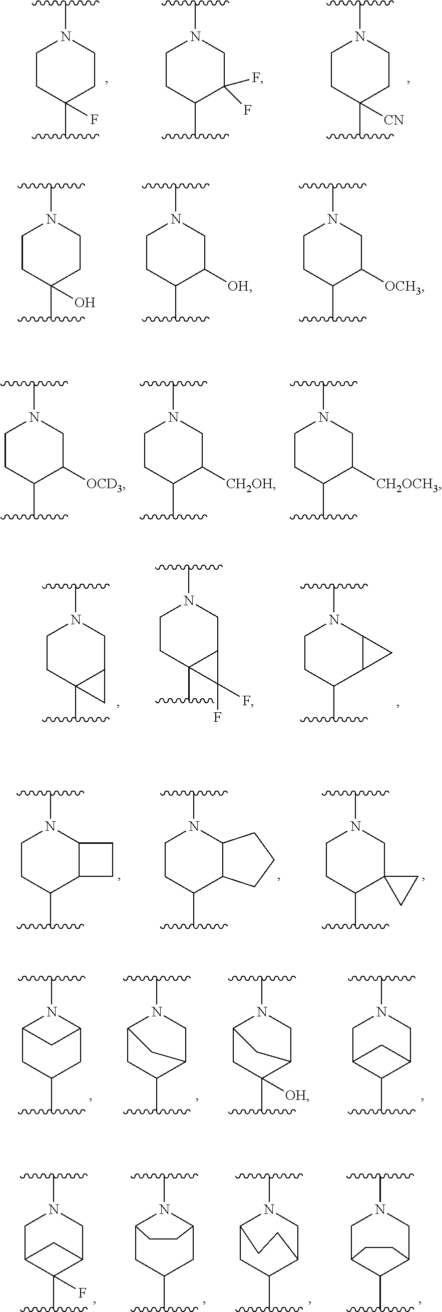

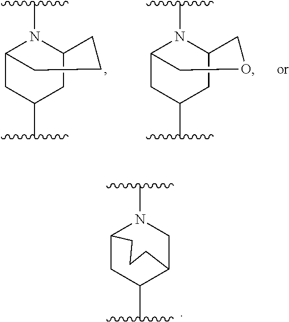 [SMILES: *C1CC2CCCC(C1)N2* ; *C1CC2COCC(C1)N2* ; *C1CC2CCCC1CN2*]

Provided herein as Embodiment 110 is the compound or salt of any one of Embodiments 1-81 and 84, wherein X is

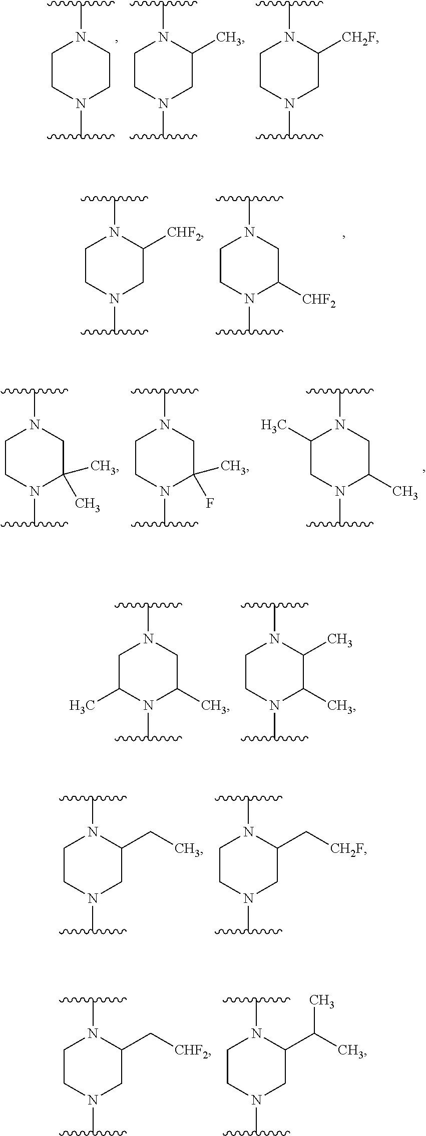

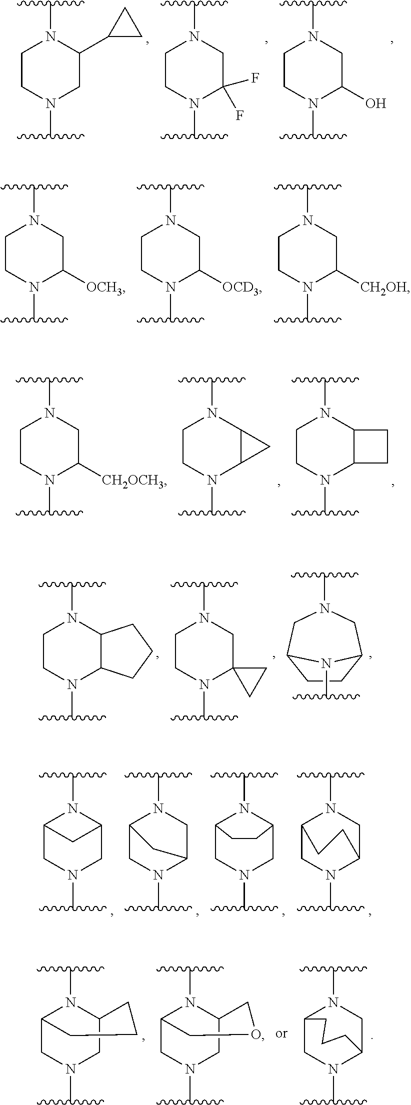

Provided herein as Embodiment 111 is the compound or salt of any one of Embodiments 1-81 and 85, wherein X is

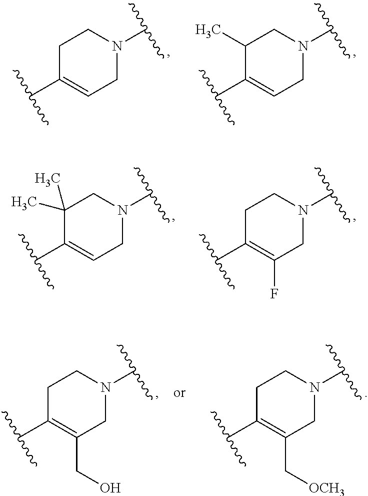

Provided herein as Embodiment 112 is the compound or salt of any one of Embodiments 1-111, wherein Z is phenyl optionally substituted with 1-4 substituents selected from halo, C₀₋₃ alkyleneCN, C₀₋₃ alkyleneOH, C₀₋₃ alkylene-C₁₋₄ alkoxy, C₀₋₃ alkylene-C₁₋₄ thioalkoxy, and

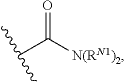

wherein each R^{N1} independently H or CH₃.

Provided herein as Embodiment 113 is the compound or salt of Embodiment 112, wherein each of the 1-4 substituents independently is selected from F, Cl, CN, OCH₃, SCH₃, CH₂OH, and

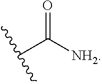 [SMILES: *C(N)=O]

Provided herein as Embodiment 114 is the compound or salt of Embodiment 113, wherein Z is

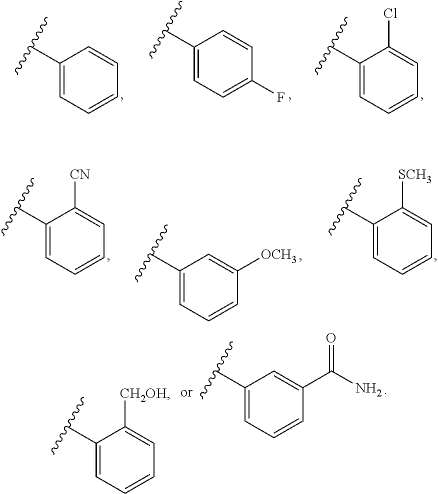

Provided herein as Embodiment 115 is the compound or salt of any one of Embodiments 1-111, wherein Z is heteroaryl comprising 5 or 6 total ring atoms and 1-3 heteroatoms selected from N, O, and S, wherein the heteroaryl is optionally substituted with 1-4 substituents.

Provided herein as Embodiment 116 is the compound or salt of Embodiment 115, wherein the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl.

Provided herein as Embodiment 117 is the compound or salt of Embodiment 116, wherein the heteroaryl is imidazolyl, thiazoyl, isothiazolyl, oxazolyl, isoxazolyl, or triazolyl.

Provided herein as Embodiment 118 is the compound or salt of Embodiment 116, wherein the heteroaryl is pyridyl, pyridazinyl, pyrimidinyl, or pyrazinyl.

Provided herein as Embodiment 119 is the compound or salt of any one of Embodiments 115-118, wherein the heteroaryl is substituted with 1-4 substituents selected, each of which is selected from the group consisting of halo, CN, C₁₋₆ alkyl, C₁₋₆ haloalkyl, C₂₋₆ alkenyl, C₂₋₆ haloalkenyl, C₀₋₆ alkylene-OH, C₀₋₆ alkylene-C₁₋₃ alkoxy, C₀₋₆ alkylene-N(R^{N1})₂ wherein each R^{N1} independently is H or C₁₋₃ alkyl, C₀₋₂ alkylene-cycloalkyl having 3-6 total ring atoms, C₀₋₂ alkylene-heterocycloalkyl having 5 or 6 total ring atoms and 1-3 heteroatoms selected from N, O, and S, and C₁₋₂ alkylene-phenyl; wherein each of the alkyl, alkenyl, C₀₋₆ alkylene-C₁₋₃ alkoxy, cycloalkyl, heterocycloalkyl, and phenyl substituents is optionally substituted with 1-3 substituents independently selected from deuterium, halo, OH, CH₃, OCH₃, and OCD₃.

Provided herein as Embodiment 120 is the compound or salt of Embodiment 119, wherein each of the 1-4 substituents independently is selected from the group consisting of Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃, CH₂CH₂CH₂OCH₃, CH₂CH₃CH₂OCD₃, CH(CH₃)CH₂OCH₃, CH(CH₃)CH₂OCD₃, C(CH₃)₂CH₂OCH₃, C(CH₃)₂CH₂OCD₃, CH₂CH(CH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

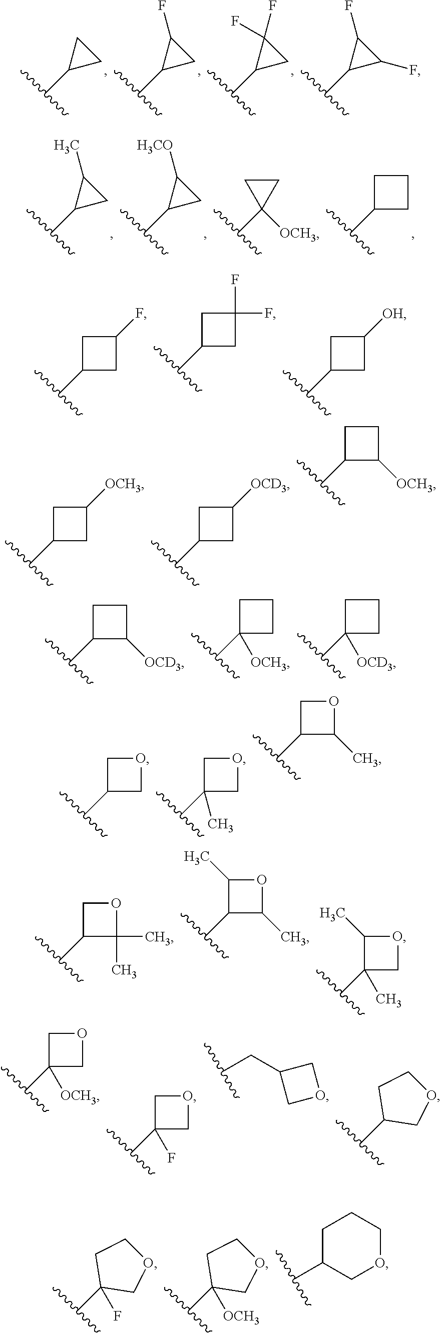

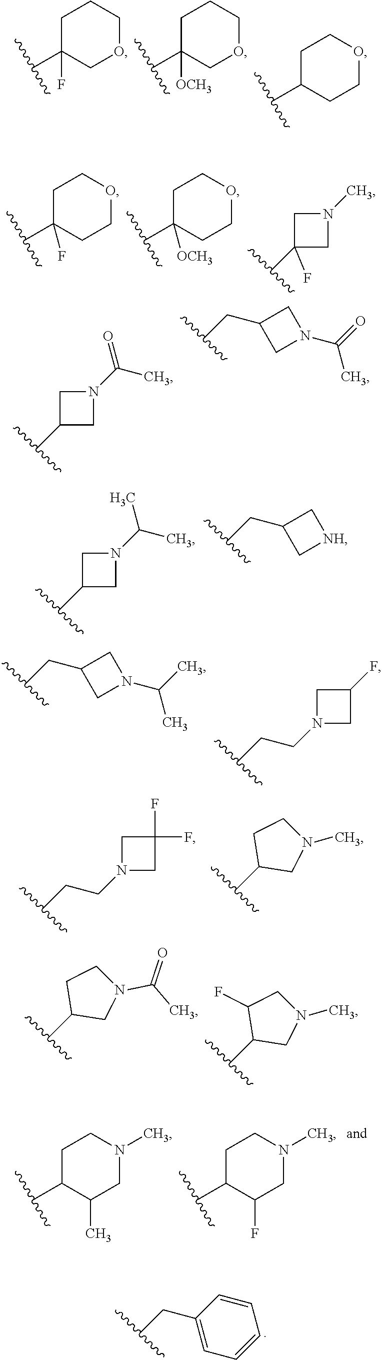

Provided herein as Embodiment 121 is the compound or salt of Embodiment 120, wherein each of the 14 substituents independently is CH₃, CH(CH₃)₂, C(CH₃)₂OH, CH₂OCD₃, CH₂CH₂OCH₃, CF₂CH₂OCH₃, CH₂CH₂OCD₃, CH₂CH₂CH₂OCH₃, CH₂CH(CH₃)OCH₃, CH₂C(CH₃)₂OCH₃,

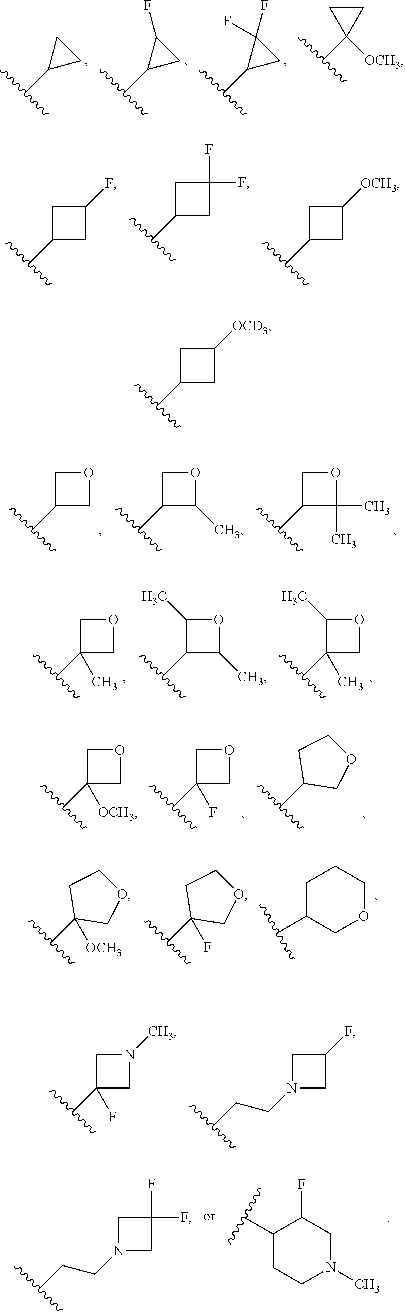

Provided herein as Embodiment 122 is the compound or salt of any one of Embodiments 115-117, wherein Z is

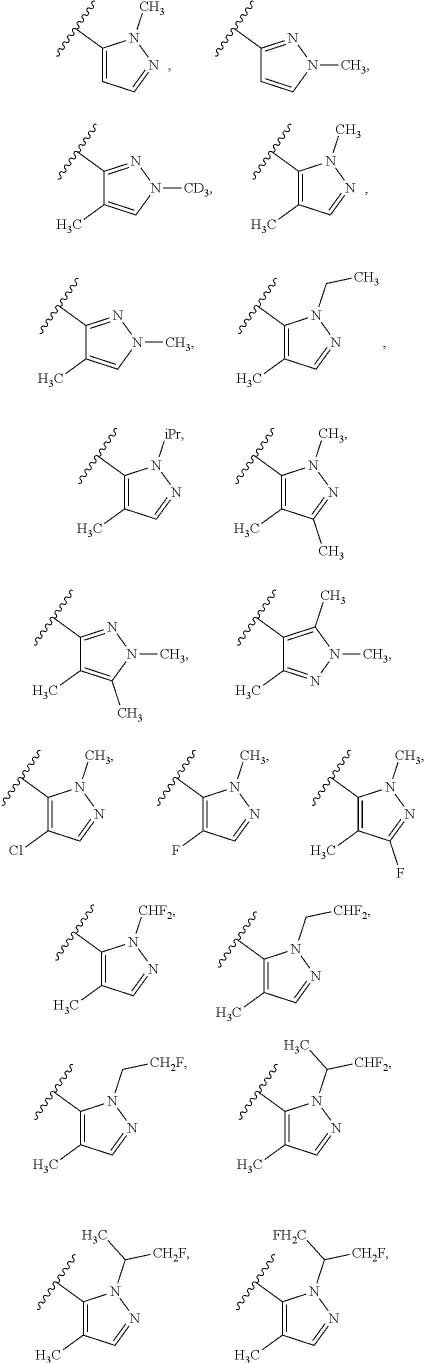

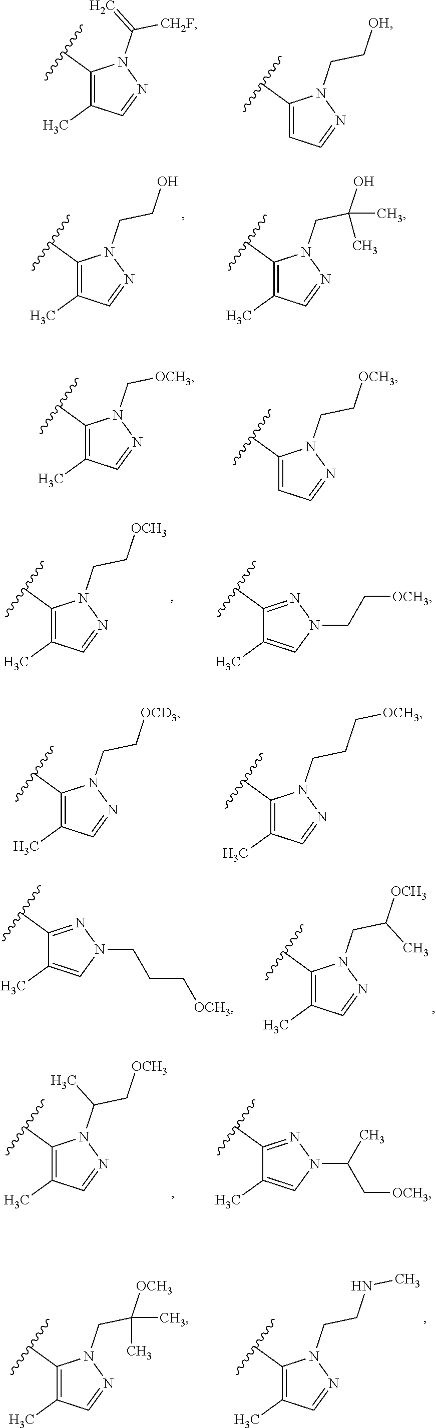

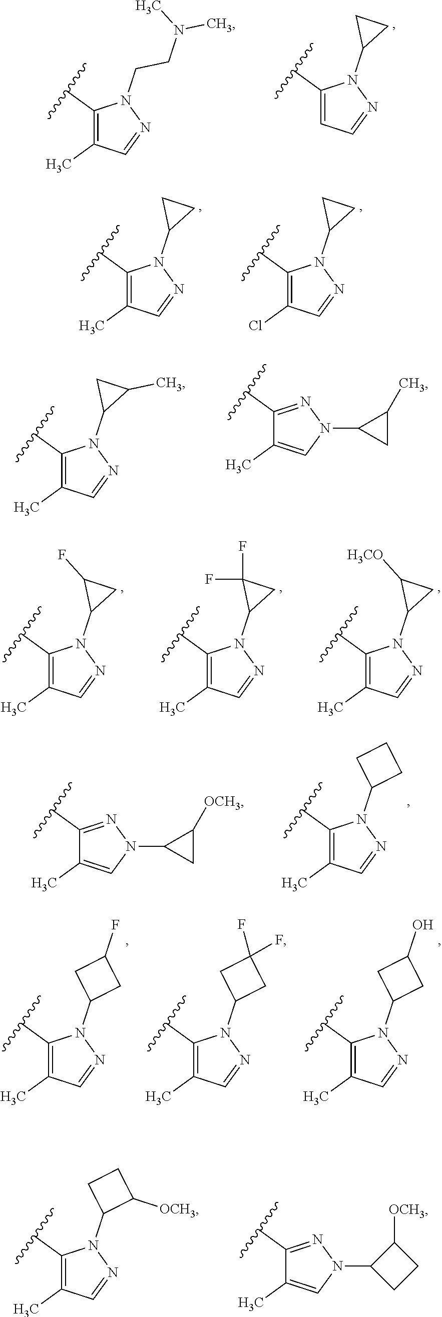

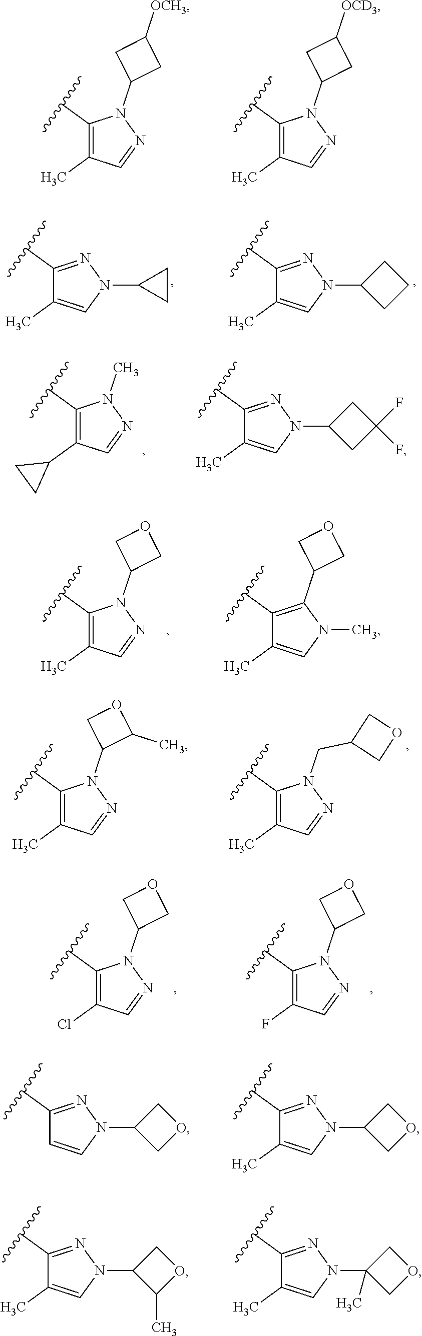

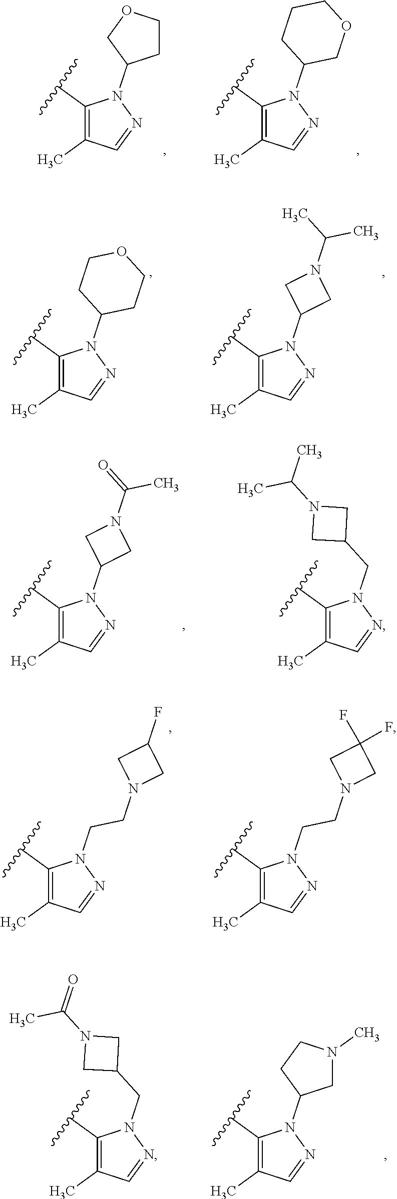

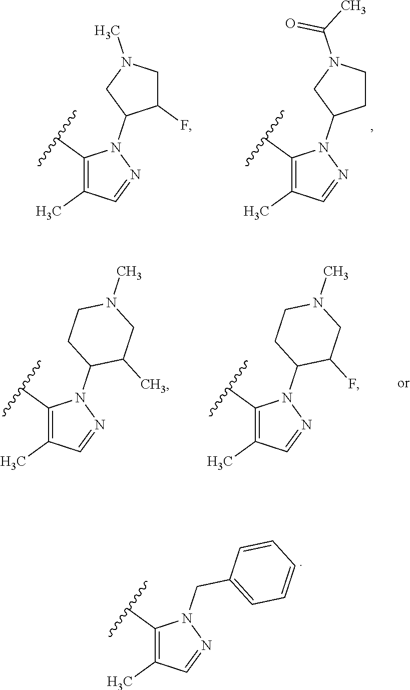 [SMILES: *c1c(C)cnn1C1CN(C)CC1F ; *c1c(C)cnn1C1CCN(C(C)=O)C1 ; *c1c(C)cnn1C1CCN(C)CC1C ; *c1c(C)cnn1C1CCN(C)CC1F ; "or" ; *c1c(C)cnn1Cc1ccccc1]

Provided herein as Embodiment 123 is the compound or salt of Embodiment 122, wherein Z is

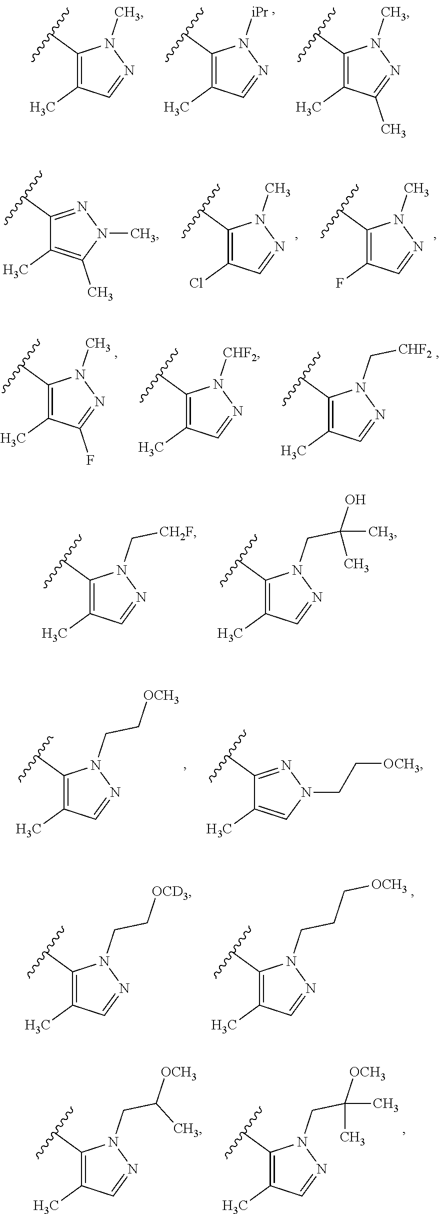

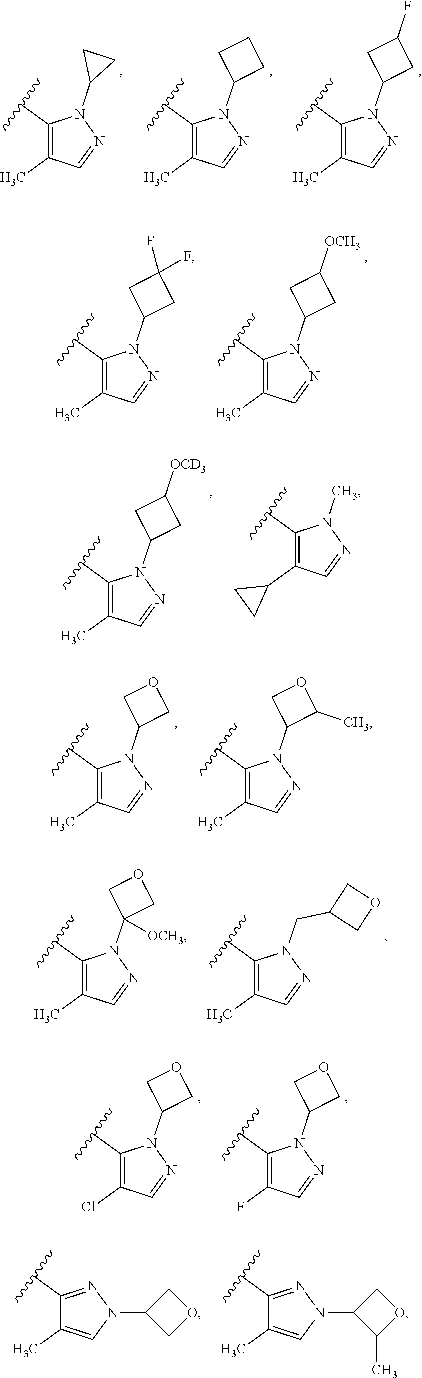

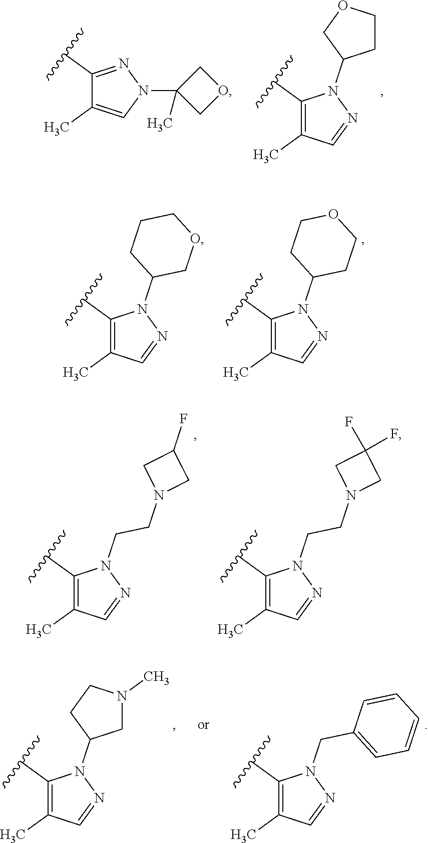

Provided herein as Embodiment 124 is the compound or salt of Embodiment 123, wherein Z is

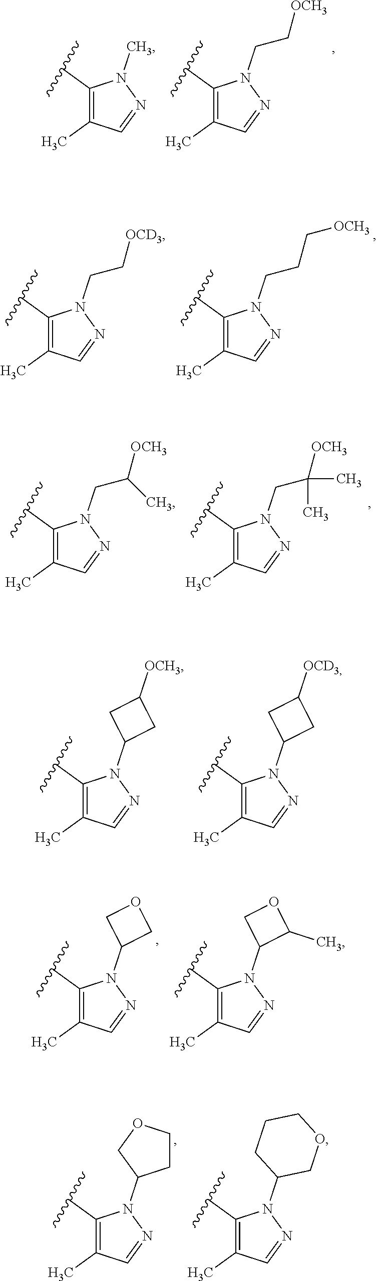

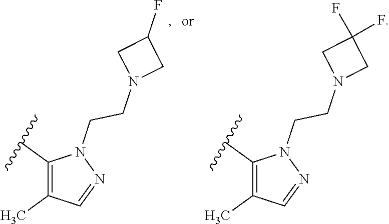 [SMILES: *c1c(C)cnn1CCN1CC(F)C1 ; *c1c(C)cnn1CCN1CC(F)(F)C1]

Provided herein as Embodiment 125 is the compound or salt of any one of Embodiments 115-117, wherein Z is

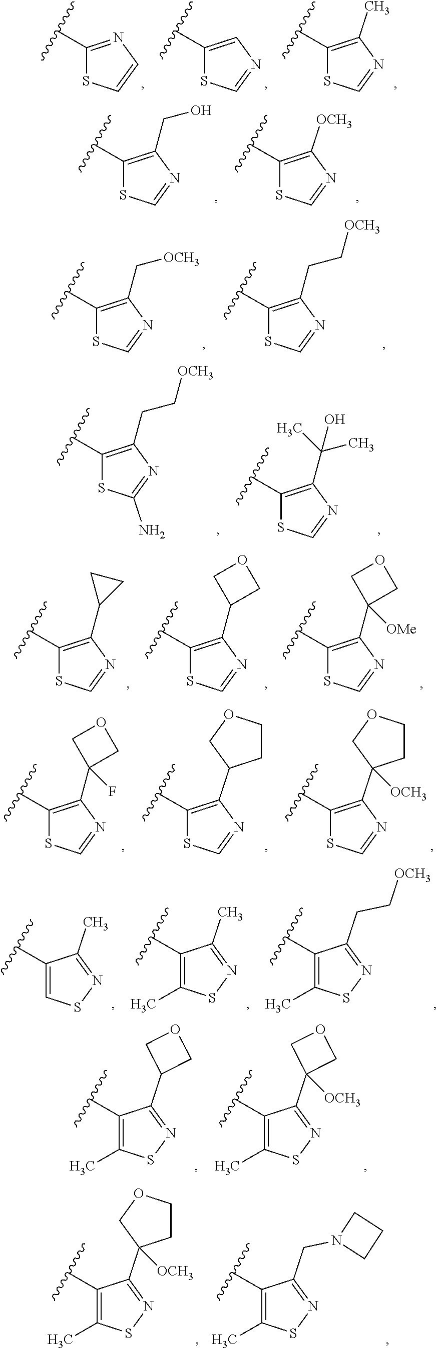

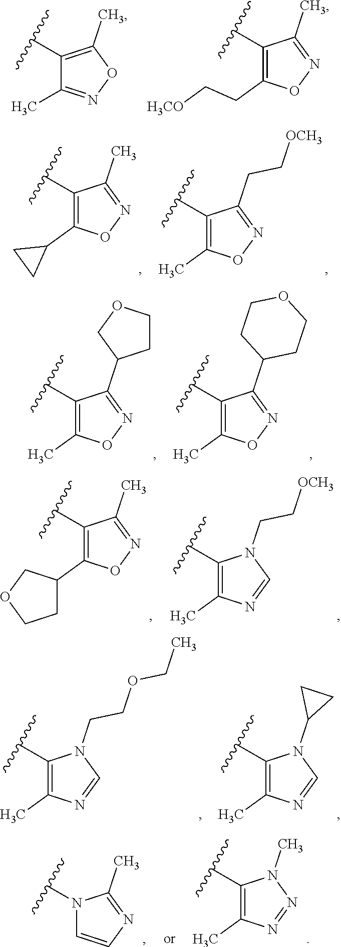

Provided herein as Embodiment 126 is the compound or salt of Embodiment 125, wherein Z is

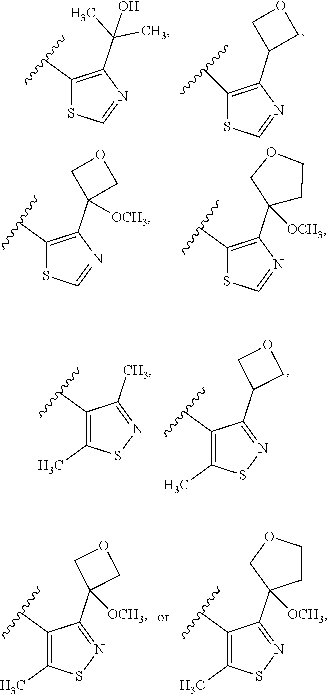

Provided herein as Embodiment 127 is the compound or salt of Embodiment 118, wherein Z is

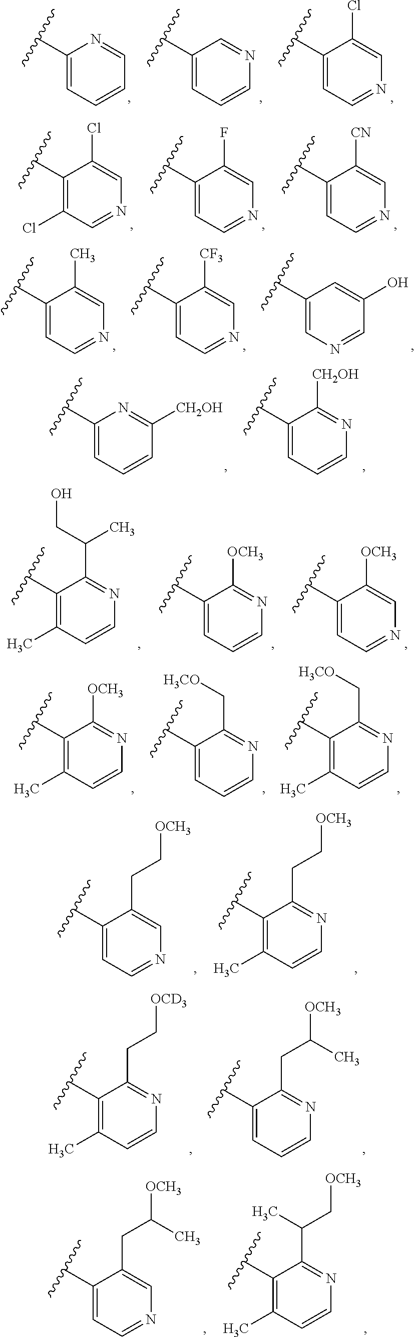

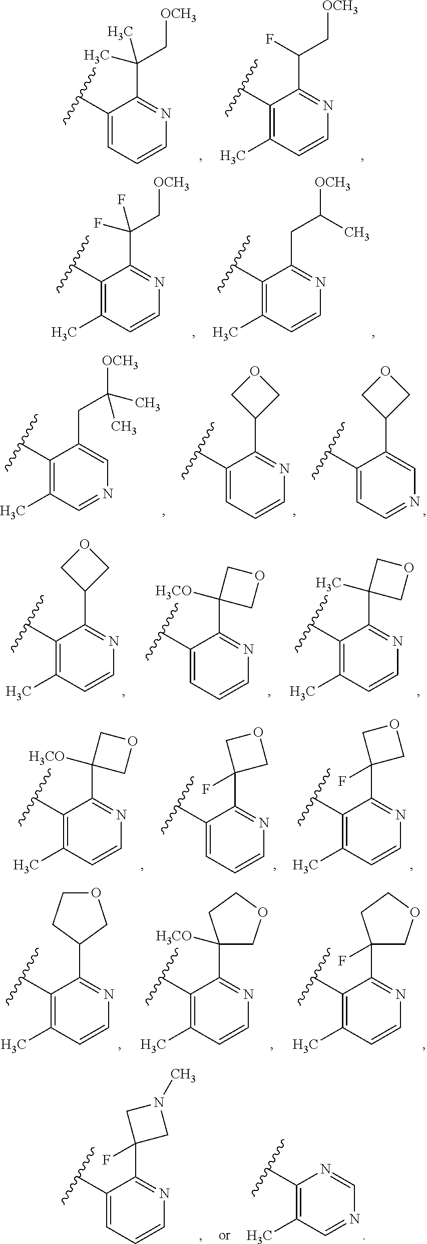

Provided herein as Embodiment 128 is the compound or salt of Embodiment 127, wherein Z is

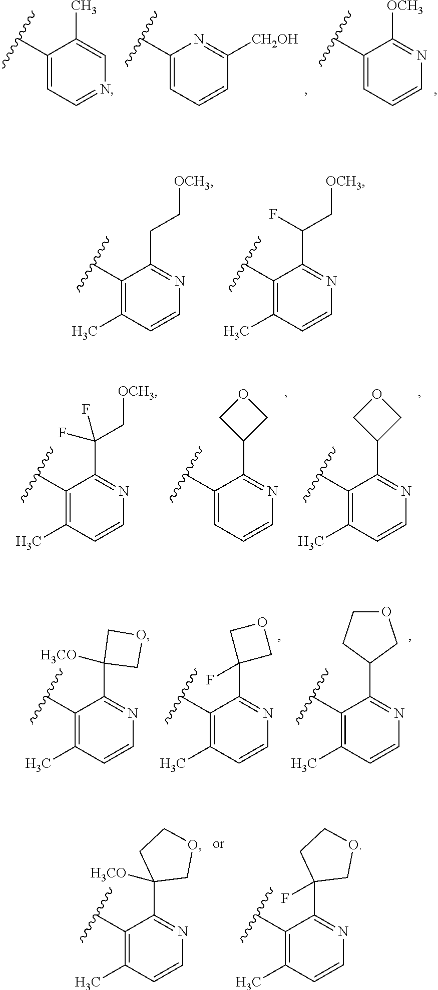

Provided herein as Embodiment 129 is the compound or salt of any one of Embodiments 1-111, wherein Z is a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein the bicyclic ring is optionally substituted with 14 substituents.

Provided herein as Embodiment 130 is the compound or salt of Embodiment 129, wherein the heteroaryl ring is pyridyl, pyridylvinyl, pyrimidinyl, or pyrazinyl, the cycloalkyl ring is cyclopentyl or cyclohexyl; and the heterocycloalkyl ring is pyrrolidinyl, tetrahydrofuranyl, tetrahydropyranyl, or tetrahydrothiophenyl.

Provided herein as Embodiment 131 is the compound or salt of Embodiment 129 or 130, wherein the bicyclic ring is substituted with 1-4 substituents selected from halo, CN, C₁₋₆ alkyl, C₁₋₆ haloalkyl, C₀₋₆ alkylene-OH, and C₀₋₆ alkylene-C₁₋₃ alkoxy.

Provided herein as Embodiment 132 is the compound or salt of any one of Embodiments 129-131, wherein Z is

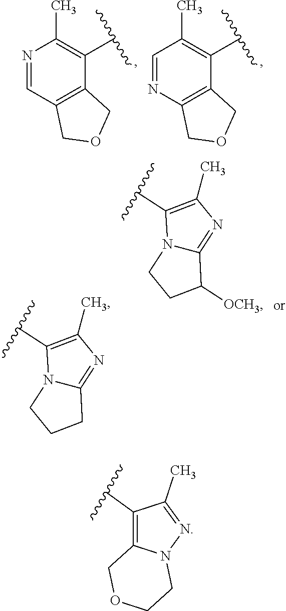

Provided herein as Embodiment 133 is the comp und or salt of Embodiment 1, wherein:

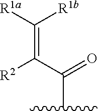

is

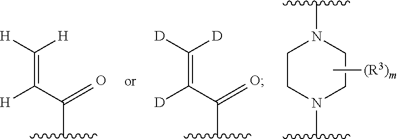

is

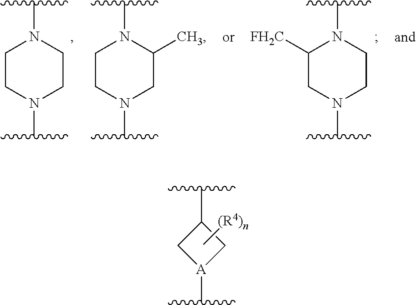

is

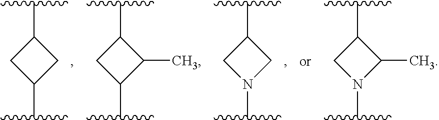

Provided herein as Embodiment 134 is the compound of Embodiment 133, wherein

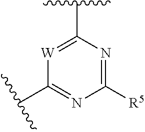

is

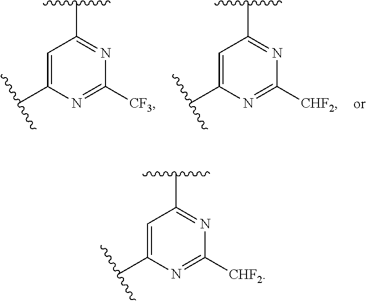 [SMILES: *c1cc(*)nc(C(F)(F)F)n1 ; *c1cc(*)nc(C(F)F)n1 ; *c1cc(*)nc(C(F)F)n1]

Provided herein as Embodiment 135 is the compound or salt of Embodiment 133 or 134, wherein X is

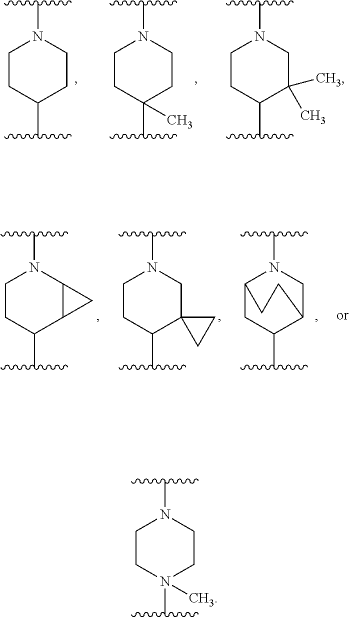

Provided herein as Embodiment 136 is the compound or salt of any one of Embodiments 133-135, wherein Z is

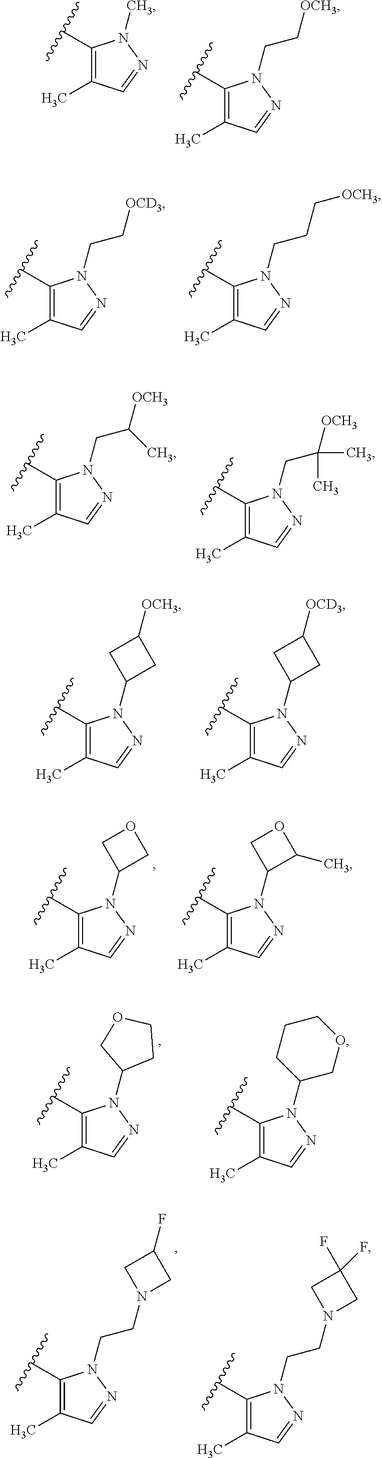

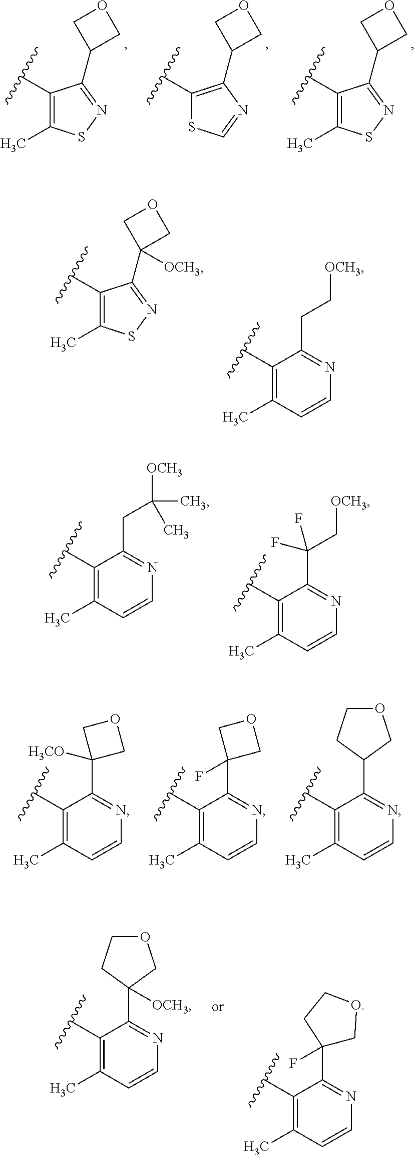

Provided herein as Embodiment 137 is the compound or salt of Embodiment 1, wherein the compound is a compound of Formula (I′):

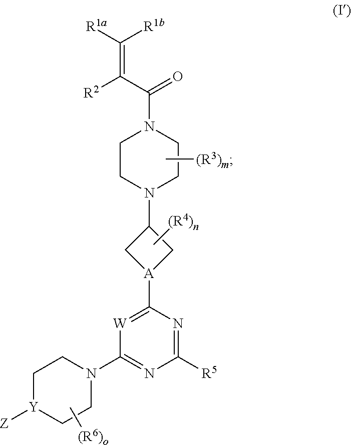

### Formula (IA):

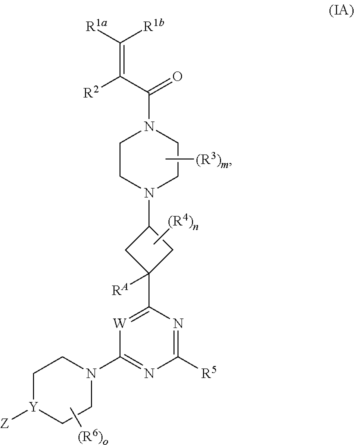

wherein R^{A} is H, halo, CN, C₁₋₃ alkyl, C₁₋₃ haloalkyl, C₀₋₃ alkyleneOH, or C₀₋₃ alkylene-C₁₋₄ alkoxy; Formula (IB):

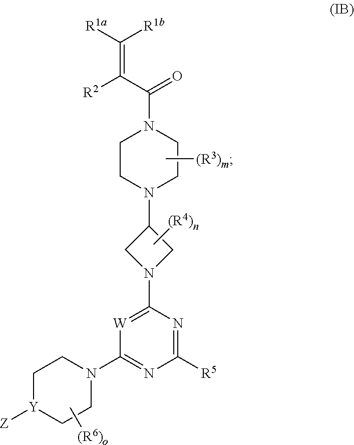

### Formula (IC):

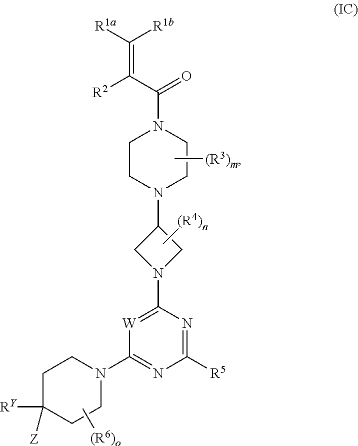

wherein R^{Y} is H, halo, CN, C₁₋₃ alkyl, C₁₋₃ haloalkyl, C₀₋₃ alkyleneOH, or C₀₋₃ alkylene-C₁₋₄ alkoxy; Formula (ID):

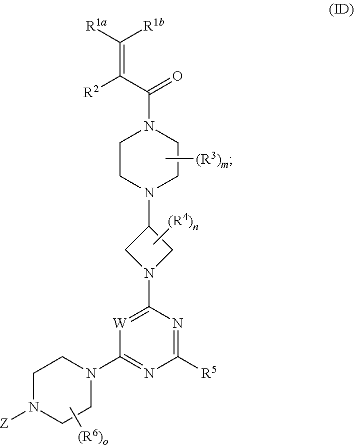

### Formula (IE):

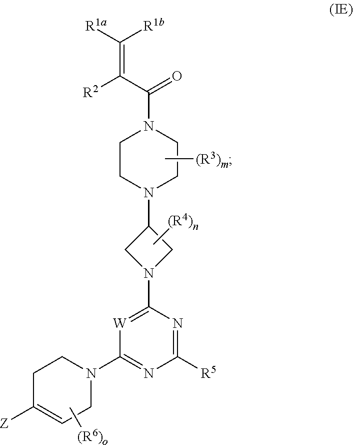

### or Formula (IF):

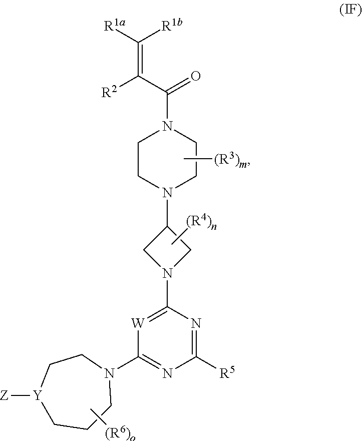

or a pharmaceutically acceptable salt of any of the foregoing.

Provided herein as Embodiment 138 is the compound of Embodiment 1, wherein the compound is a compound listed m Table A, or a pharmaceutically acceptable salt thereof.

Provided herein as Embodiment 139 is the compound of Embodiment 1, wherein the compound is a compound listed in Table E, or a pharmaceutically acceptable salt thereof.

Provided herein as Embodiment 140 is a pharmaceutical composition comprising the compound or salt of any one of Embodiments 1-139 and a pharmaceutically acceptable excipient.

Provided herein as Embodiment 141 is the compound or salt of any one of Embodiments 1-139, or the pharmaceutical composition of Embodiment 140 for use as a medicament.

Provided herein as Embodiment 142 is the compound or salt of any one of Embodiments 1-139 or the pharmaceutical composition of Embodiment 140 for use in treating cancer.

Provided herein as Embodiment 143 is the compound or salt of any one of Embodiments 1-139 or the pharmaceutical composition of Embodiment 140 for use in treating cancer, wherein one or more cells express KRAS G12C mutant protein.

Provided herein as Embodiment 144 is the compound, salt, or pharmaceutical composition for use of Embodiment 142 or 143, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 145 is a use of a compound or salt of any one of Embodiments 1-139 or the pharmaceutical composition of Embodiment 140 in the preparation of a medicament for treating cancer.

Provided herein as Embodiment 146 is a use of a compound or salt of any one of Embodiments 1-139 or the pharmaceutical composition of Embodiment 140 in the preparation of a medicament for treating cancer, wherein one or more cells express KRAS G12C mutant protein.

Provided herein as Embodiment 147 is the use of Embodiment 145 or 146, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thy roid cancer, leukemia, melanoma, or a solid tumor.

Provided herein as Embodiment 148 is a method of treating cancer in a subject in need thereof, the method comprising administering to the subject a therapeutically effective amount of the compound or salt of any one of Embodiments 1-139, or the pharmaceutical composition of Embodiment 140.

Provided herein as Embodiment 149 is the method of Embodiment 148, wherein one or more cells express KRAS G12C mutant protein.

Provided herein as Embodiment 150 is the method of Embodiment 148 or 149, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thy roid cancer, leukemia, melanoma, or a solid tumor.

Provided herein as Embodiment 151 is the method of Embodiment 150, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, or a solid tumor.

Provided herein as Embodiment 152 is the method of Embodiment 151, wherein the cancer is non-small cell lung cancer.

Provided herein as Embodiment 153 is the method of Embodiment 151, wherein the cancer is colorectal cancer.

Provided herein as Embodiment 154 is the method of Embodiment 151, wherein the cancer is pancreatic cancer.

Provided herein as Embodiment 155 is the method of Embodiment 151, wherein the cancer is solid tumor.

Provided herein as Embodiment 156 is the method according to any one of Embodiments 148-155, wherein the subject has a cancer that was determined to have one or more cells expressing the KRAS G12C mutant protein prior to administration of the compound, salt, or pharmaceutical composition.

Provided herein as Embodiment 157 is the method according to any one of Embodiments 148-156, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents.

Provided herein as Embodiment 158 is the compound or salt of any one of Embodiments 1-139, wherein the compound or salt has an IC₅₀ value of less than 1 μM in the coupled exchange assay disclosed herein in the BIOLOGICAL EVALUATION section.

### Alternative Embodiments

Provided herein as Embodiment 1 is a compound of Formula (I):

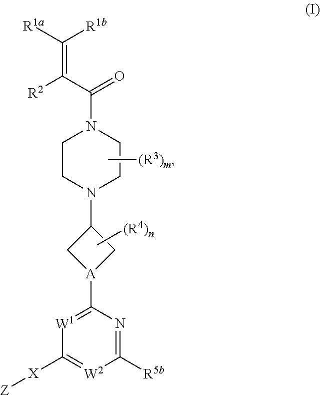

or a pharmaceutically acceptable salt thereof.
wherein:
m is 0, 1, 2, 3, or 4;
n is 0, 1, or 2;
A is N, CH, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy;
each of W¹ and W² independently is N, CH, C-halo, C—CN, C—C₁₋₃ alkyl, C—C₂₋₃ alkenyl, C—C₂₋₃ alkynyl, C—C₁₋₃ haloalkyl, C—C₀₋₃ alkyleneOH, or C—C₀₋₃ alkylene-C₁₋₄ alkoxy, wherein each of the alkenyl and alkynyl is optionally substituted with 1 or more substituents;
X is heterocycloalkyl or heterocycloalkenyl, each having 4-7 total ring atoms and 1-3 heteroatoms selected from N, O, and S, wherein each of the heterocycloalkyl and heterocycloalkenyl is optionally substituted with 1 or more substituents;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to a ring having 5 or 6 total ring atoms and 0, 1, or 2 heteroatoms selected from N, O, and S; wherein each of the phenyl, heteroaryl, and bicyclic rings is optionally substituted with 1 or more substituents;
each of R^{1a}, R^{1b}, and R² independently is H, D, halo, C₁₋₄ alkyl, C₁₋₄ haloalkyl, C₁₋₂ alkylene-OH, C₀₋₂ alkylene-C₁₋₄ alkoxy, C₀₋₂ alkylene-C₁₋₄ haloalkoxy, C₀₋₂ alkylene-CN, C₀₋₂ alkylene-N(R^{N1})₂, C₁₋₂ alkylene-heterocycloalkyl having 3-6 total ring atoms and 1 or 2 heteroatoms selected from N, O, and S, or R^{1b} and R², together with the carbon atoms to which they are attached, form

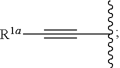

each R³ independently is C₁₋₃ alkyl, C₁₋₃ haloalkyl,

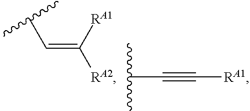

C₀₋₃ alkyleneCN, C₀₋₃ alkyleneOH, C₀₋₃ alkylene-C₁₋₃ alkoxy, oxo, spiro-cycloalkyl having 3-7 total ring atoms, spiro-cycloalkenyl having 4-7 total ring atoms, spiro-heterocycloalkyl having 3-7 total ring atoms and 1 or 2 heteroatoms selected from N, O and S, spiro-heterocycloalkenyl having 4-7 total ring atoms and 1 or 2 heteroatoms selected from N, O and S, or two adjacent R³, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1 or 2 heteroatoms selected from N, O, and S, or a fused heterocycloalkenyl ring having 4-7 total ring atoms and 1 or 2 heteroatoms selected from N, O, and S; each R⁴ independently is C₁₋₃ alkyl, C₁₋₃ haloalkyl, C₀₋₃ alkyleneCN, C₁₋₃ alkyleneOH, C₁₋₃ alkylene-C₁₋₃ alkoxy, oxo, spiro-cycloalkyl having 3-7 total ring atoms, or spiro-heterocycloalkyl having 4-7 total ring atoms and 1 or 2 heteroatoms selected from N, O and S;
R⁵ is C₁₋₃ haloalkyl, C₁₋₆ alkyl, C₂₋₄ alkenyl, C₂₋₄ alkynyl, halo, C₁₋₃ alkoxy, C₁₋₃ thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing independently is optionally substituted with 1 or more substituents;
each of R^{A1} and R^{A2} independently is H, C₁₋₃ alkyl, C₁₋₃ haloalkyl, or cycloalkyl having 3-5 total ring atoms; and
each R^{N1} independently is H or C₁₋₄ alkyl.

Provided herein as Embodiment 2 is the compound or salt of Embodiment 1, wherein at least one of R^{1a}, R^{1b}, and R² is H or D.

Provided herein as Embodiment 3 is the compound or salt of Embodiment 2, wherein each of R^{1a}, R^{1b}, and R² independently is H or D.

Provided herein as Embodiment 4 is the compound or salt of Embodiment 3, wherein each of R^{1a}, R^{1b}, and R² independently is H.

Provided herein as Embodiment 5 is the compound or salt of Embodiment 3, wherein each of R^{1a}, R^{1b}, and R² independently is D.

Provided herein as Embodiment 6 is the compound or salt of Embodiment 1 or 2, wherein at least one of R^{1a}, R^{1b}, and R² is halo.

Provided herein as Embodiment 7 is the compound or salt of Embodiment 6, wherein R^{1a} is halo and each of R^{1b} and R² is H.

Provided herein as Embodiment 8 is the compound or salt of Embodiment 6 or 7, wherein each halo independently is Br, Cl, or F.

Provided herein as Embodiment 9 is the compound or salt of Embodiment 1 or 2, wherein at least one of R^{1a}, R^{1b}, and R² is C₁₋₄ alkyl or C₁₋₄ haloalkyl.

Provided herein as Embodiment 10 is the compound or salt of Embodiment 9, wherein at least one of R^{1a}, R^{1b}, and R² is CH₃, CH₂F, CHF₂, or CF₃.

Provided herein as Embodiment 11 is the compound or salt of Embodiment 1 or 2, wherein at least one of R^{1a}, R^{1b}, and R² is C₁₋₂ alkylene-OH, C₀₋₂ alkylene-C₁₋₄ alkoxy, C₀₋₂ alkylene-C₁₋₄ haloalkoxy, C₀₋₂ alkylene-CN, or C₀₋₂ alkylene-N(R^{N1})₂.

Provided herein as Embodiment 12 is the compound or salt of Embodiment 11, wherein each R^{N1} independently is H or CH₃.

Provided herein as Embodiment 13 is the compound or salt of Embodiment 12, wherein each R^{N1} independently is H.

Provided herein as Embodiment 14 is the compound or salt of Embodiment 11 or 12, wherein at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂.

Provided herein as Embodiment 15 is the compound or salt of Embodiment 1 or 2, wherein at least one of R^{1a}, R^{1b}, and R² is C₁₋₂ alkylene-heterocycloalkyl having 3-6 total ring atoms and 1 or 2 heteroatoms selected from N, O, and S.

Provided herein as Embodiment 16 is the compound or salt of Embodiment 15, wherein the heterocycloalkyl is aziridinyl, azetidinyl, pyrrolidinyl, piperidinyl, or morpholinyl.

Provided herein as Embodiment 17 is the compound or salt of Embodiment 16, wherein at least one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl.

Provided herein as Embodiment 18 is the compound or salt of Embodiment 1, wherein R^{1b} and R², together with the carbon atoms to which they are attached, form

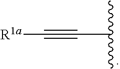

Provided herein as Embodiment 19 is the compound or salt of Embodiment 1, wherein

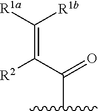

is

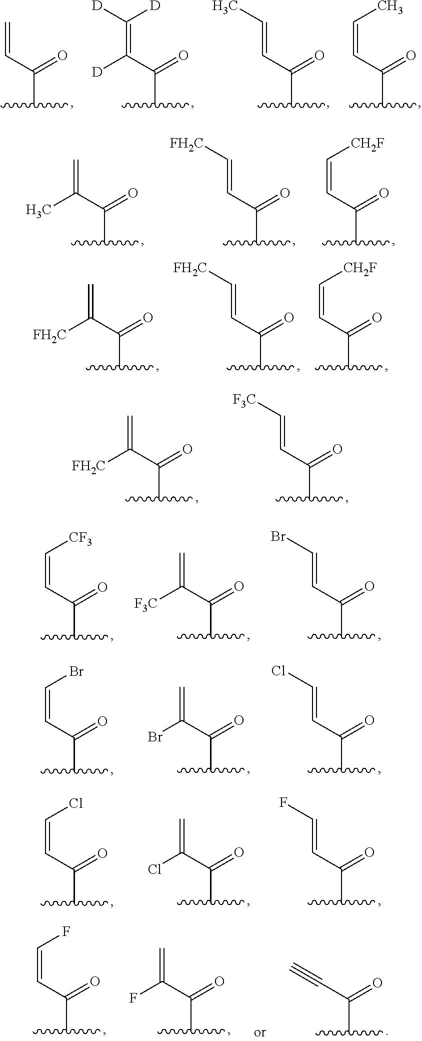

Provided herein as Embodiment 20 is the compound or salt of Embodiment 19, wherein

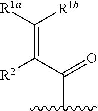

is

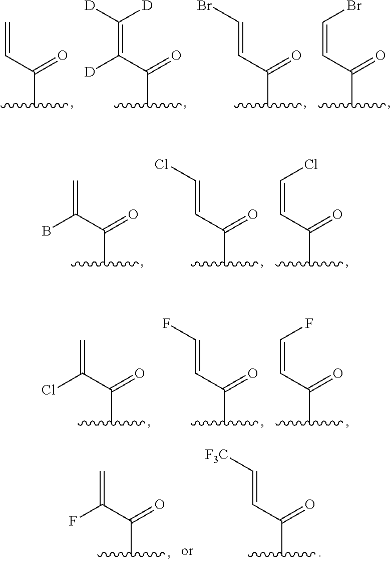

Provided herein as Embodiment 21 is the compound or salt of Embodiment 20, wherein

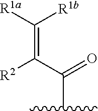

is

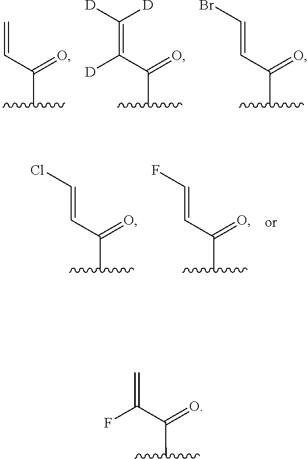

Provided herein as Embodiment 22 is the compound or salt of Embodiment 21, wherein

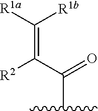

is

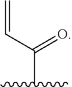 [SMILES: *C(=O)C=C]

Provided herein as Embodiment 23 is the compound or salt of any one of Embodiments 1-22, wherein m is 0.

Provided herein as Embodiment 24 is the compound or salt of any one of Embodiments 1-22, wherein m is 1.

Provided herein as Embodiment 25 is the compound or salt of an one of Embodiments 1-22, wherein m is 2.

Provided herein as Embodiment 26 is the compound or salt of any one of Embodiments 1-22, wherein m is 3.

Provided herein as Embodiment 27 is the compound or salt of any one of Embodiments 1-22, wherein m is 4.

Provided herein as Embodiment 28 is the compound or salt of any one of Embodiments 1-22, wherein

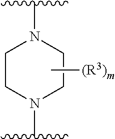

is deuterated.

Provided herein as Embodiment 29 is the compound or salt of Embodiment 28, wherein

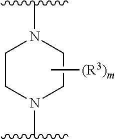

is

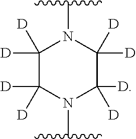

Provided herein as Embodiment 30 is the compound of salt of any one of Embodiments 24-27, wherein at least one R³ is C₁₋₃ alkyl or C₁₋₃ haloalkyl.

Provided herein as Embodiment 31 is the compound or salt of Embodiment 30, wherein at least one R³ is CH₃, CH₂CH₃, CF₃, CHF₂, or CH₂F.

Provided herein as Embodiment 32 is the compound or salt of Embodiment 31, wherein at least one R³ is CH₃.

Provided herein as Embodiment 33 is the compound or salt of any one of Embodiments 24-27, wherein at least one R³ is

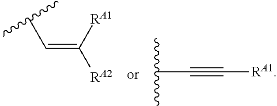

Provided herein as Embodiment 34 is the compound or salt of Embodiment 33, wherein each of R^{A1} and R^{A2} independently is H, CH₃, CH₂F, CHF₂, CF₃, CH₂CH₃, CH₂CH₂CH₃, CH(CH₃)₂, cyclopropyl, or cyclobutyl.

Provided herein as Embodiment 35 is the compound of Embodiment 33 or 34, wherein at least one R³ is

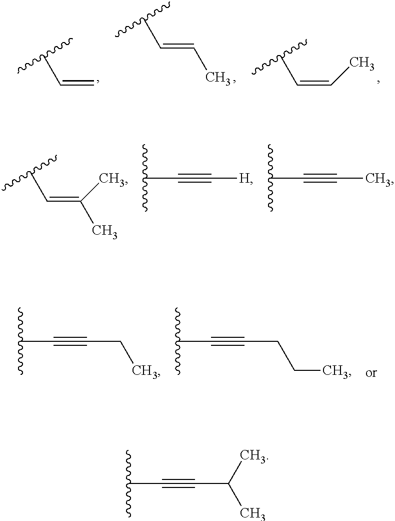

Provided herein as Embodiment 36 is the compound or salt of any one of Embodiments 24-27, wherein at least one R³ is C₀₋₃ alkyleneCN.

Provided herein as Embodiment 37 is the compound or salt of Embodiment 36, wherein at least one R³ is CN or CH₂CN.

Provided herein as Embodiment 38 is the compound or salt of any one of Embodiments 24-27, wherein at least one R³ is C₀₋₃ alkyleneOH or C₀₋₃ alkylene-C₁₋₃ alkoxy.

Provided herein as Embodiment 39 is the compound or salt of Embodiment 38, wherein at least one R³ is OH, CH₂OH, CH₂CH₂OH, OCH₃, CH₂OCH₃, or CH₂CH₂OCH₃.

Provided herein as Embodiment 40 is the compound or salt of any one of Embodiments 24-27, wherein at least one R³ is oxo.

Provided herein as Embodiment 41 is the compound or salt of any one of Embodiments 24-27, wherein at least one R³ is spiro-cycloalkyl having 3-7 total ring atoms or spiro-heterocycloalkyl having 3-7 total ring atoms and 1 or 2 heteroatoms selected from N, O and S.

Provided herein as Embodiment 42 is the compound or salt of Embodiment 41, wherein at least one R³ is spiro-cyclopropyl, spiro-cyclobutyl, spiro-oxetanyl, or spiro-tetrahydrofuranyl.

Provided herein as Embodiment 43 is the compound or salt of any one of Embodiments 24-27, wherein at least one R³ is spiro-cycloalkenyl having 4-7 total ring atoms or spiro-heterocycloalkenyl having 4-7 total ring atoms and 1 or 2 heteroatoms selected from N, O and S.

Provided herein as Embodiment 44 is the compound or salt of any one of Embodiments 24-27, wherein two adjacent R³, together with the atoms to which they are attached, form a fused cycloalkyl ring having 3-7 total ring atoms or a fused heterocycloalkyl ring having 3-7 total atoms and 1 or 2 heteroatoms selected from N, O, and S.

Provided herein as Embodiment 45 is the compound or salt of Embodiment 44, wherein two adjacent R³, together with the atoms to which they are attached, form a fused cyclopropyl ring or a fused cyclobutyl ring.

Provided herein as Embodiment 46 is the compound or salt of any one of Embodiments 24-27, wherein two adjacent R³, together with the atoms to which they are attached, form a fused cycloalkenyl ring having 4-7 total ring atoms or a fused heterocycloalkenyl ring having 4-7 total ring atoms and 1 or 2 heteroatoms selected from N, O and S.

Provided herein as Embodiment 47 is the compound or salt of any one of Embodiments 24-27, wherein each R³ independently is CH₃, CH₂CH₃, CF₃, CHF₂, CH₂F, CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, spiro-tetrahydrofuranyl, or two adjacent R³, together with the atoms to which they are attached, form fused-cyclopropyl, or fused-cyclobutyl.

Provided herein as Embodiment 48 is the compound or salt of any one of Embodiments 1-22, wherein m is 0; or m is 1 and R³ is CH₃, CF₃, CHF₂, CH₂F, CN, CH₂CN, CH₂OH, CH₂OCH₃, or spiro-oxetanyl.

Provided herein as Embodiment 49 is the compound or salt of Embodiment 43, wherein m is 0; or m is 1 and R³ is CH₃.

Provided herein as Embodiment 50 is the compound or salt of any one of Embodiments 1-22, wherein

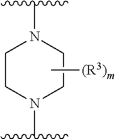

is

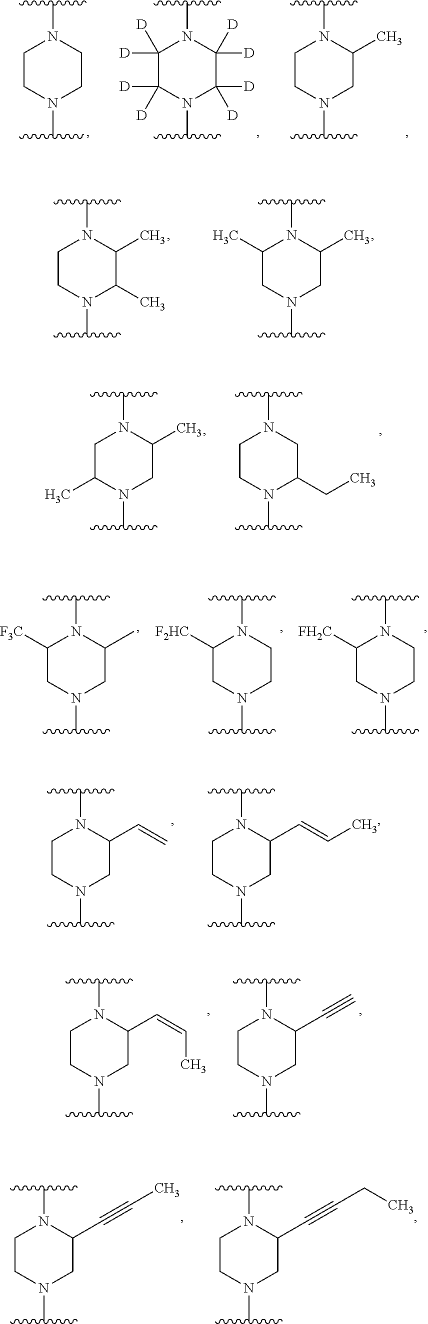

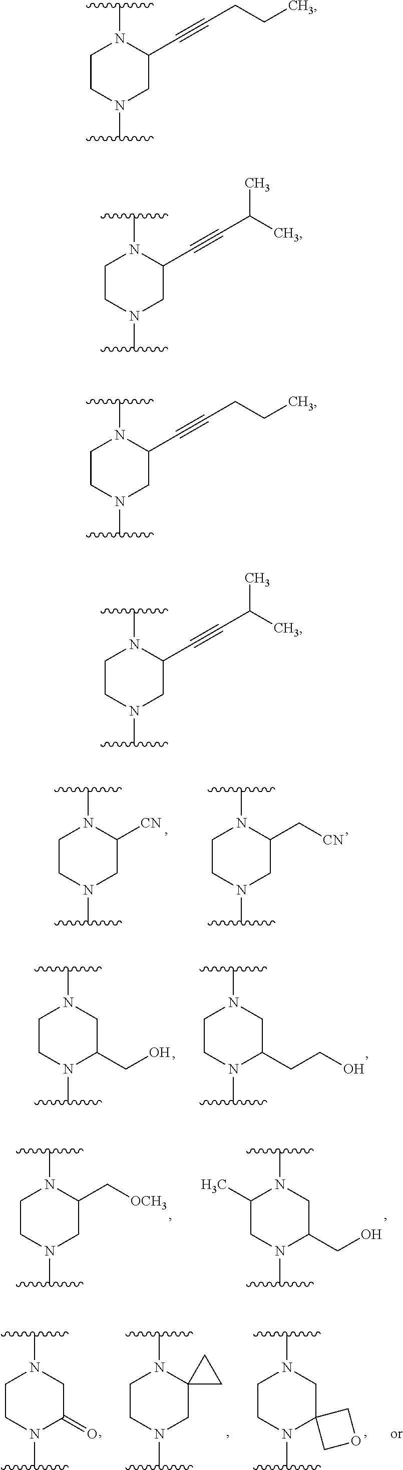

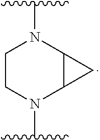 [SMILES: *N1CCN(*)C2CC21]

Provided herein as Embodiment 51 is the compound or salt of Embodiment 50, wherein

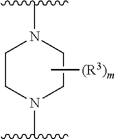

is

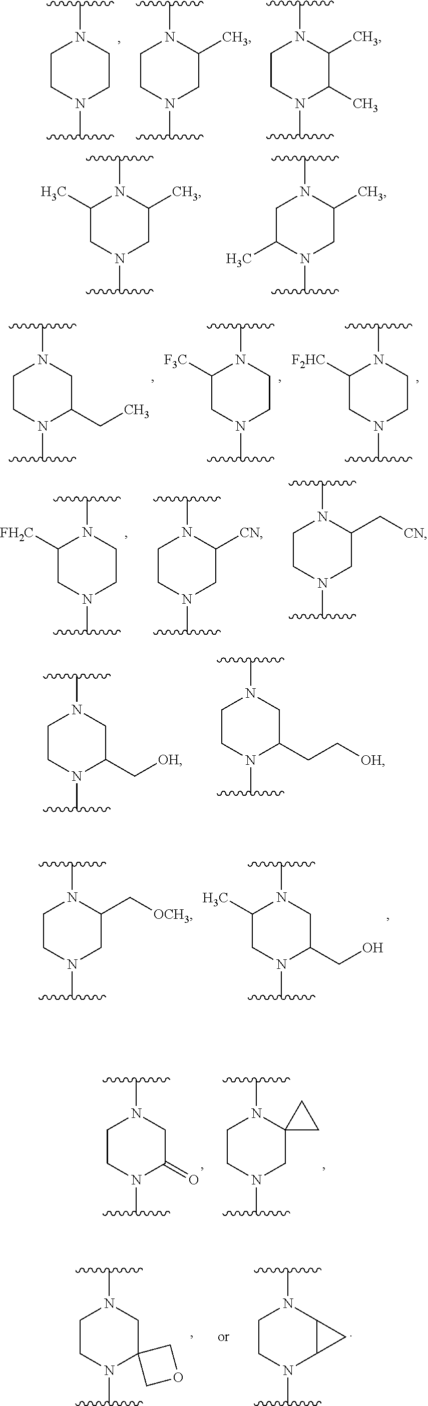

Provided herein as Embodiment 52 is the compound or salt of Embodiment 51, wherein

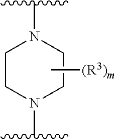

is

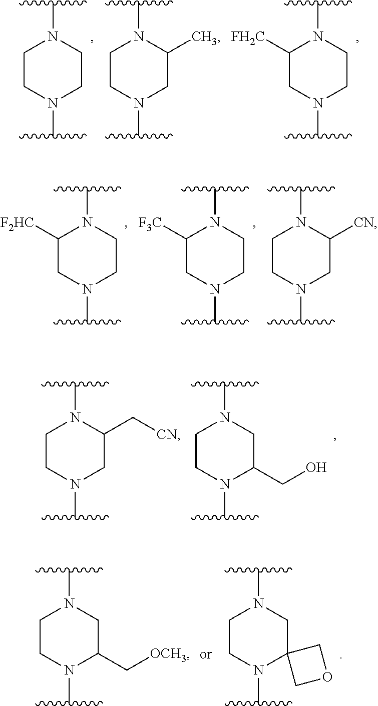

Provided herein as Embodiment 53 is the compound or salt of Embodiment 51, wherein

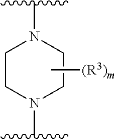

is

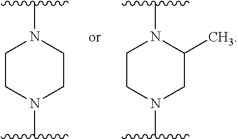 [SMILES: *N1CCN(*)CC1 ; *N1CCN(*)C(C)C1]

Provided herein as Embodiment 54 is the compound or salt of any one of Embodiments 1-53, wherein A is N, CH, or C—C₁₋₃ alkyl.

Provided herein as Embodiment 55 is the compound or salt of any one of Embodiments 1-53, wherein A is N.

Provided herein as Embodiment 56 is the compound or salt of any one of Embodiments 1-53, wherein A is CH, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy.

Provided herein as Embodiment 57 is the compound or salt of Embodiment 54 or 56, wherein A is CH.

Provided herein as Embodiment 58 is the compound or salt of Embodiment 56, wherein A is C—F, C—Cl, or C—CN.

Provided herein as Embodiment 59 is the compound or salt of Embodiment 54 or 56, wherein A is C—C₁₋₃ alkyl or C—C₁₋₃ haloalkyl.

Provided herein as Embodiment 60 is the compound or salt of Embodiment 59, wherein A is C—CH₃, C—CH₂F, C—CHF₂, or C—CF₃.

Provided herein as Embodiment 61 is the compound or salt of Embodiment 56, wherein A is C—C₀₋₃ alkyleneOH or C—C₀₋₃ alkylene-C₁₋₄ alkoxy.

Provided herein as Embodiment 62 is the compound or salt of Embodiment 61, wherein A is C—OH, C—CH₂OH, C—OCH₃, or C—CH₂OCH₃.

Provided herein as Embodiment 63 is the compound or salt of any one of Embodiments 1-62, wherein n is 0.

Provided herein as Embodiment 64 is the compound or salt of any one of Embodiments 1-62, wherein n is 1.

Provided herein as Embodiment 65 is the compound or salt of any one of Embodiments 1-62, wherein n is 2.

Provided herein as Embodiment 66 is the compound or salt of Embodiment 64 or 65, wherein at least one R⁴ is C₁₋₃ alkyl or C₁₋₃ haloalkyl.

Provided herein as Embodiment 67 is the compound or salt of Embodiment 66, wherein at least one R⁴ is CH₃, CH₂CH₃, CH₂CH₂CH₃, CH(CH₃)₂, CF₃, CHF₂, or CH₂F.

Provided herein as Embodiment 68 is the compound or salt of Embodiment 67, wherein at least one R⁴ is CH₃.

Provided herein as Embodiment 69 is the compound or salt of Embodiment 64 or 65, wherein at least one R⁴ is C₀₋₃ alkyleneCN.

Provided herein as Embodiment 70 is the compound or salt of Embodiment 69, wherein at least one R⁴ is CN or CH₂CN.

Provided herein as Embodiment 71 is the compound or salt of Embodiment 64 or 65, wherein at least one R⁴ is C₁₋₃ alkyleneOH or C₁₋₃ alkylene-C₁₋₃ alkoxy.

Provided herein as Embodiment 72 is the compound or salt of Embodiment 71, wherein at least one R⁴ is CH₂OH, CH₂CH₂OH, CH₂OCH₃, or CH₂CH₂OCH₃.

Provided herein as Embodiment 73 is the compound or salt Embodiment 64 or 65, wherein at least one R³ is oxo.

Provided herein as Embodiment 74 is the compound or salt of Embodiment 64 or 65, wherein at least one R³ is spiro-cycloalkyl having 3-7 total ring atoms or spiro-heterocycloalkyl having 4-7 total ring atoms and 1 or 2 heteroatoms selected from N, O and S.

Provided herein as Embodiment 75 is the compound or salt of Embodiment 74, wherein at least one R⁴ is spiro-cyclopropyl, spiro-cyclobutyl, or spiro-oxetanyl.

Provided herein as Embodiment 76 is the compound or salt of Embodiment 54 or 55, wherein

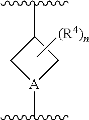

is

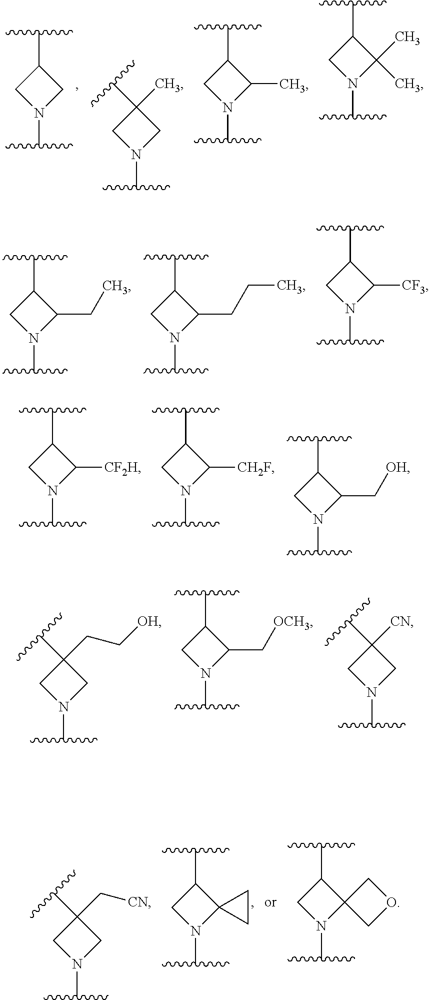

Provided herein as Embodiment 77 is the compound or salt of Embodiment 76, wherein

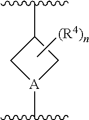

is

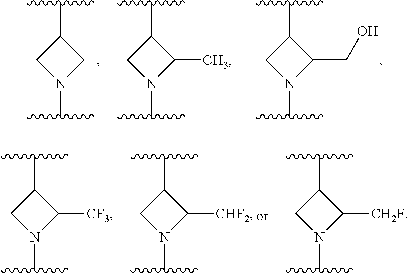

Provided herein as Embodiment 78 is the compound or salt of Embodiment 77, wherein

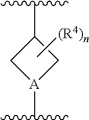

is

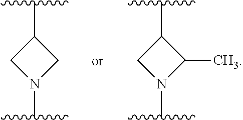 [SMILES: *C1CN(*)C1 ; "or" ; *C1CN(*)C1C]

Provided herein as Embodiment 79 is the compound or salt of any one of Embodiments 54, 56, or 57, wherein

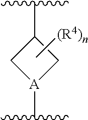

is

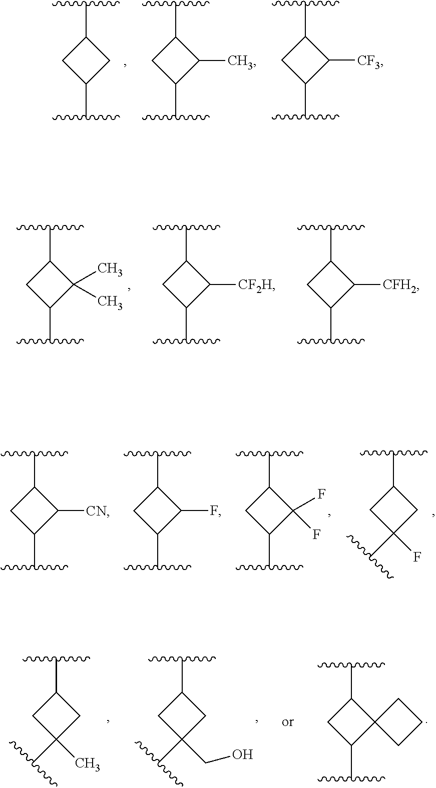

Provided herein as Embodiment 80 is the compound or salt of any one of Embodiments 1-79, wherein W¹ is N.

Provided herein as Embodiment 81 is the compound or salt of any one of Embodiments 1-79, wherein W¹ is CH.

Provided herein as Embodiment 82 is the compound or salt of any one of Embodiments 1-79, wherein W¹ is C—F, C—Cl, or C—CN.

Provided herein as Embodiment 83 is the compound or salt of any one of Embodiments 1-79, wherein W¹ is C—C₁₋₃ alkyl or C—C₁₋₃ haloalkyl.

Provided herein as Embodiment 84 is the compound or salt of Embodiment 83, wherein W¹ is C—CH₃, C—CH₂CH₃, C—CH₂F, C—CHF₂, or C—CF₃.

Provided herein as Embodiment 85 is the compound or salt of Embodiment 84, wherein W¹ is C—CH₃ or C—CH₂CH₃.

Provided herein as Embodiment 86 is the compound or salt of any one of Embodiments 1-79, wherein W¹ is C—C₂₋₃ alkenyl or C—C₂₋₃ alkynyl, and each of the alkenyl and alkynyl is optionally substituted with 1 or more substituents.

Provided herein as Embodiment 87 is the compound or salt of Embodiment 86, wherein each of the C—C₂₋₃ alkenyl and C—C₂₋₃ alkynyl is unsubstituted.

Provided herein as Embodiment 88 is the compound or salt of Embodiment 86, wherein each of the C—C₂₋₃ alkenyl and C—C₁₋₃ alkynyl is substituted with 1-3 substituents, and each substituent independently is halo, C₁₋₃ haloalkyl, C₀₋₃ alkyleneOH, or C₀₋₃ alkyleneC₁₋₄ alkoxy.

Provided herein as Embodiment 89 is the compound or salt of Embodiment 88, wherein W¹ is C—CH═CH₂, C—C(OH)═CH₂, C—CH═CH(OH), or C—CCH.

Provided herein as Embodiment 90 is the compound or salt of any one of Embodiments 1-79, wherein W¹ is C—C₀₋₃ alkyleneOH or C—C₀₋₃ alkylene-C₁₋₄ alkoxy.

Provided herein as Embodiment 91 is the compound or salt of Embodiment 90, wherein W¹ is C—OH, C—CH₂OH, C—OCH₃, or C—CH₂OCH₃.

Provided herein as Embodiment 92 is the compound or salt of any one of Embodiments 1-91, wherein W² is N.

Provided herein as Embodiment 93 is the compound or salt of any one of Embodiments 1-91, wherein W² is CH.

Provided herein as Embodiment 94 is the compound or salt of any one of Embodiments 1-91, wherein W² is C—F, C—Cl, or C—CN.

Provided herein as Embodiment 95 is the compound or salt of any one of Embodiments 1-91, wherein W² is C—C₁₋₃ alkyl or C—C₁₋₃ haloalkyl.

Provided herein as Embodiment 96 is the compound or salt of Embodiment 95, wherein W² is C—CH₃, C—CH₂CH₃, C—CH₂F, C—CHF₂, or C—CF₃.

Provided herein as Embodiment 97 is the compound or salt of any one of Embodiments 1-91, wherein W² is C—C₂₋₃ alkenyl or C—C₂₋₃ alkynyl, and each of the alkenyl and alkynyl is optionally substituted with 1 or more substituents.

Provided herein as Embodiment 98 is the compound or salt of Embodiment 97, wherein each of the C—C₂₋₃ alkenyl and C—C₂₋₃ alkynyl is unsubstituted.

Provided herein as Embodiment 99 is the compound or salt of Embodiment 97, wherein each of the C—C₂₋₃ alkenyl and C—C₂₋₃ alkynyl is substituted with 1-3 substituents, and each substituent independently is halo, C₁₋₃ haloalkyl, C₀₋₃ alkyleneOH, or C₀₋₃ alkyleneC₁₋₄ alkoxy.

Provided herein as Embodiment 100 is the compound or salt of Embodiment 97, wherein W² is C—CH═CH₂, C—C(OH)═CH₂, C—CH═CH(OH), or C—CCH.

Provided herein as Embodiment 101 is the compound or salt of any one of Embodiments 1-91, wherein W² is C—C₀₋₃ alkyleneOH or C₀₋₃ alkylene-C₁₋₄ alkoxy.

Provided herein as Embodiment 102 is the compound or salt of Embodiment 101, wherein W² is C—OH, C—CH₂OH, C—OCH₃, or C—CH₂OCH₃.

Provided herein as Embodiment 103 is the compound or salt of any one of Embodiments 1-79, wherein each of W¹ and W² independently is N, CH, or C—CH₃.

Provided herein as Embodiment 104 is the compound or salt of Embodiment 103, wherein W¹ is CH and W² is N, CH, or C—CH₃.

Provided herein as Embodiment 105 is the compound or salt of Embodiment 103, wherein W² is N and W¹ is N, CH, or C—CH₃.

Provided herein as Embodiment 106 is the compound or salt of Embodiment 103, wherein W¹ is CH and W² is N.

Provided herein as Embodiment 107 is the compound or salt of any one of Embodiments 1-106, wherein R⁵ is C₁₋₃ haloalkyl.

Provided herein as Embodiment 108 is the compound or salt of Embodiment 107, wherein R⁵ is CF₃, CF₂H, CFH₂, or CF₂CH₃.

Provided herein as Embodiment 109 is the compound or salt of Embodiment 108, wherein R⁵ is CF₃.

Provided herein as Embodiment 110 is the compound or salt of Embodiment 108, wherein R⁵ is CF₂H.

Provided herein as Embodiment 111 is the compound or salt of any one of Embodiments 1-106, wherein R⁵ is Br, Cl, or F.

Provided herein as Embodiment 112 is the compound or salt of any one of Embodiments 1-106, wherein R⁵ is C₁₋₃ alkoxy or C₁₋₃ thioalkoxy.

Provided herein as Embodiment 113 is the compound or salt of Embodiment 112, wherein R⁵ is OCH₃, or SCH₃.

Provided herein as Embodiment 114 is the compound or salt of any one of Embodiments 1-106, wherein R³ is C₁₋₆ alkyl, C₂₋₄ alkenyl, or C₂₋₄ alkynyl, and each of the foregoing is optionally substituted with 1-3 substituents.

Provided herein as Embodiment 115 is the compound or salt of Embodiment 114, wherein the alkyl is CH₃, CH₂CH₃, CH₂CH₃CH, or CH(CH₃)₂; the alkenyl is CH═CH₂ or CH═CHCH₃; and the alkynyl is

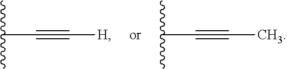

wherein each of the foregoing is optionally substituted with 1-3 substituents.

Provided herein as Embodiment 116 is the compound or salt of Embodiment 114 or 115, wherein R⁵ is unsubstituted.

Provided herein as Embodiment 117 is the compound or salt of Embodiment 114 or 115, wherein R is substituted with 1-3 substituents, and each substituent independently is C₁₋₃ haloalkyl, C₀₋₆ alkylene-OH, C₀₋₆ alkylene-C₁₋₅ alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl.

Provided herein as Embodiment 118 is the compound or salt of Embodiment 128, wherein each substituent independently is CH₃, CF₃, CF₂H, CFH₂, OH, OCH₃. OCF₃, CH₂OH, CH₂OCH₃, cyclopropyl, cyclobutyl, or phenyl.

Provided herein as Embodiment 119 is the compound or salt of Embodiment 114, wherein R³ is CH₃, CH₂CH₃, CH₂CH₂CH₃, CH(CH₃)₂,

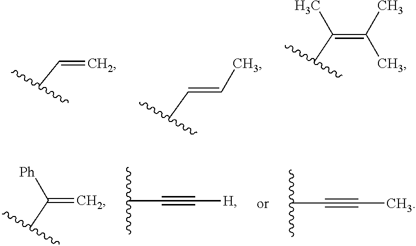

Provided herein as Embodiment 120 is the compound or salt of any one of Embodiments 1-106, wherein R³ is cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing is optionally substituted with 1-3 substituents.

Provided herein as Embodiment 121 is the compound or salt of Embodiment 120, wherein each of the cycloalkyl, cycloalkenyl, heterocycloalkyl, and heterocycloalkenyl is unsubstituted.

Provided herein as Embodiment 122 is the compound or salt of Embodiment 121, wherein each of the cycloalkyl, cycloalkenyl, heterocycloalkyl, and heterocycloalkenyl is substituted with 1-3 substituents, and each substituent independently is halo, C₁₋₃ alkyl, C₁₋₃ haloalkyl, C₀₋₆ alkylene(OH), or C₀₋₆ alkylene-C₁₋₃ alkoxy.

Provided herein as Embodiment 123 is the compound or salt of any one of Embodiments 1-106, —CH₂ wherein R⁵ is CH₃, CF₃, CF₂H, CFH₂, CH₂CH₃, CH₂CH₂CH₃, CH(CH₃)₂,

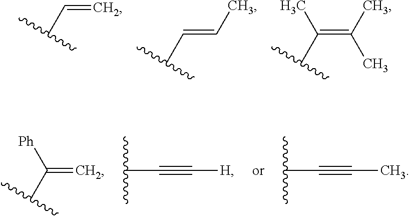

Provided herein as Embodiment 124 is the compound or salt of any one of Embodiments 1-64, wherein W¹ is CH, W² is N, and R⁵ is CF₃, CF₂H, or CFH₂.

Provided herein as Embodiment 125 is the compound or salt of any one of Embodiments 1-79, wherein

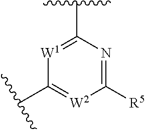

is

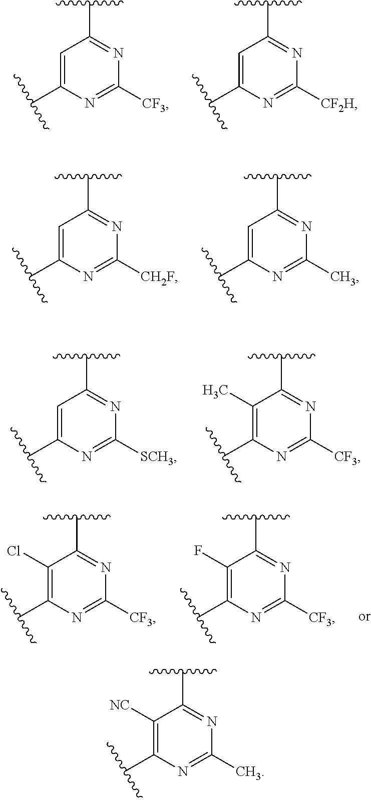

Provided herein as Embodiment 126 is the compound or salt of Embodiment 125, wherein

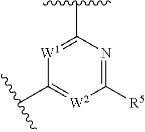

is

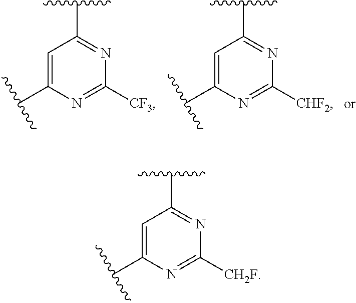

Provided herein as Embodiment 127 is the compound or salt of Embodiment 126, wherein

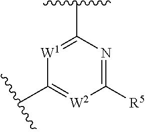

is

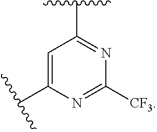 [SMILES: *c1cc(*)nc(C(F)(F)F)n1]

Provided herein as Embodiment 128 is the compound or salt of Embodiment 126, wherein

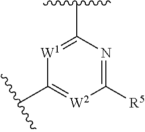

is

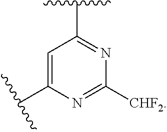 [SMILES: *c1cc(*)nc(C(F)F)n1]

Provided herein as Embodiment 129 is the compound or salt of Embodiment 1-128, wherein:
X is

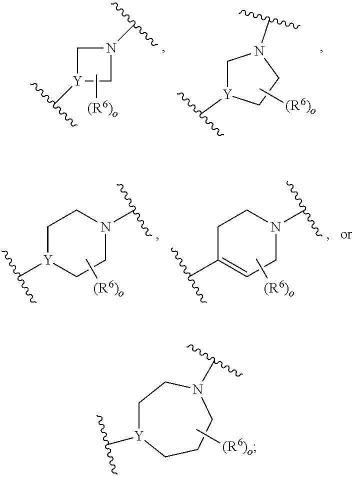

Y is N, C—H, C-halo, C—CN, C—C₁₋₃ alkyl, C—C₁₋₃ haloalkyl, C—C₀₋₃ alyleneOH, or C—C₀₋₃ alkylene-C₁₋₄ alkoxy;
o is 0, 1, 2, 3, or 4; and
each R⁶ independently is halo, CN, C₁₋₃ alkyl, C₂₋₃ alkenyl, C₁₋₃ haloalkyl, C₀₋₃ alkylene-OH, C₀₋₃ alkylene-C₁₋₃ alkoxy, deuterated C₀₋₃ alkylene-C₁₋₃ alkoxy, C₁₋₄ alkylene-N(R^{N1})₂, oxo, ═CH₂, spiro-cycloalkyl having 3-7 total atoms, spiro-cycloalkenyl having 4-7 total atoms, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two adjacent R, together with the atoms to which they are attached, form a fused cycloalkyl ring having 3-7 total ring atoms, a fused cycloalkenyl ring having 4-7 total ring atoms, a fused heterocycloalkyl ring having 4-7 total ring atoms and 1-3 heteroatoms selected from N, O, and S, or a fused heterocycloalkenyl ring having 4-7 total ring atoms and 1 or 2 heteroatoms selected from N, O, and S; or two non-adjacent R⁶ join together to form a C₁₋₃ alkylene bridge, a C₂₋₃ alkenylene bridge, a C₁₋₃ ether bridge, or a C₁₋₃ thioether bridge; or Y and an adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1-3 heteroatoms selected from N, O, and S, or a fused heterocycloalkenyl ring having 4-7 total ring atoms and 1 or 2 heteroatoms selected from N, O, and S; wherein the cycloalkyl, cycloalkenyl, heterocycloalkyl or heterocycloalkenyl of any of the foregoing is optionally substituted with 1 or more substituents, and
each R^{N1} independently is H or C₁₋₄ alkyl.

Provided herein as Embodiment 130 is the compound or salt of Embodiment 129, wherein o is 0.

Provided herein as Embodiment 131 is the compound or salt of Embodiment 129, wherein o is 1.

Provided herein as Embodiment 132 is the compound or salt of Embodiment 129, wherein o is 2.

Provided herein as Embodiment 133 is the compound or salt of Embodiment 129, wherein o is 3.

Provided herein as Embodiment 134 is the compound or salt of Embodiment 129, wherein o is 4.

Provided herein as Embodiment 135 is the compound or salt of any one of Embodiments 131-134, wherein at least one R³ is Br, Cl, F, or CN.

Provided herein as Embodiment 136 is the compound or salt of Embodiment 135, wherein at least one R⁶ is F.

Provided herein as Embodiment 137 is the compound or salt of any one of Embodiments 131-134, wherein at least one R⁶ is C₁₋₃ alkyl or C₁₋₃ haloalkyl.

Provided herein as Embodiment 138 is the compound or salt of Embodiment 137, wherein at least one R⁶ is CH₃, CH₂F, CHF₂, or CF₃.

Provided herein as Embodiment 139 is the compound or salt of any one of Embodiments 131-134, wherein at least one R⁶ is C₀₋₃ alkylene-OH, C₁₋₃ alkylene-C₁₋₃ alkoxy, deuterated C₀₋₃ alkylene-C₁₋₃ alkoxy, or C₁₋₄ alkylene-N(R^{N1})₂, and each R^{N1} independently is H or CH₃.

Provided herein as Embodiment 140 is the compound or salt of Embodiment 139, wherein at least one R⁶ is OH, CH₂OH, OCH₃, OCD₃, CH₂OCH₃, or CH₂N(CH₃)₂.

Provided herein as Embodiment 141 is the compound or salt of any one of Embodiments 131-134, wherein at least one R⁶ is oxo or ═CH₂.

Provided herein as Embodiment 142 is the compound or salt of any one of Embodiments 131-134, wherein at least one R³ is spiro-cycloalkyl having 3-7 total atoms, spiro-cycloalkenyl having 4-7 total atoms, spiro-heterocycloalkyl having 4-7 total ring atoms and 1 or 2 heteroatoms selected from N. O and S, or spiro-heterocycloalkenyl having 4-7 total ring atoms and 1 or 2 heteroatoms selected from N, O and S, wherein any of the foregoing is optionally substituted with 1 or more substituents.

Provided herein as Embodiment 143 is the compound or salt of Embodiment 142, wherein at least one R⁶ is spiro-cyclopropyl, spiro-cyclobutyl, spiro-oxetanyl, or spiro-tetrahydrofuranyl, and any of the foregoing is optionally substituted with 1 or more substituents.

Provided herein as Embodiment 144 is the compound or salt of Embodiment 143, wherein at least one R³ is spiro-cyclopropyl.

Provided herein as Embodiment 145 is the compound or salt of any one of Embodiments 132-134, wherein two adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 4-7 total ring atoms and 1-3 heteroatoms selected from N, O, and S, or a fused heterocycloalkenyl having 4-7 total ring atoms and 1 or 2 heteroatoms selected from N, O, and S; or Y and an adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1-3 heteroatoms selected from N, O, and S, or a fused heterocycloalkenyl ring having 4-7 total ring atoms and 1 or 2 heteroatoms selected from N, O, and S, wherein the cycloalkyl, cycloalkenyl, heterocycloalkyl, or heterocycloalkenyl of any of the foregoing is optionally substituted with 1 or more substituents.

Provided herein as Embodiment 146 is the compound or salt of Embodiment 145, wherein two adjacent R⁶, together with the atoms to which they are attached, form a fused cycloalkyl ring having 3-7 total ring atoms; or Y and an adjacent R⁶, together with the atoms to which they are attached, form a fused cycloalkyl ring having 3-7 total ring atoms, wherein the cycloalkyl of any of the foregoing is optionally substituted with 1 or more substituents.

Provided herein as Embodiment 147 is the compound or salt of Embodiment 145 or 146, wherein the fused cycloalkyl ring is fused-cyclopropyl, fused-cyclobutyl, or fused-cyclopentyl, and any of the foregoing is optionally substituted with 1 or more substituents.

Provided herein as Embodiment 148 is the compound or salt of any one of Embodiments 145-147, wherein the cycloalkyl, cycloalkenyl, heterocycloalkyl, or heterocycloalkenyl is unsubstituted.

Provided herein as Embodiment 149 is the compound or salt of any one of Embodiments 145-147, wherein the cycloalkyl, cycloalkenyl, heterocycloalkyl, or heterocycloalkenyl is substituted with 1 or more substituents.

Provided herein as Embodiment 150 is the compound or salt of any one of Embodiments 145-147, wherein the cycloalkyl, cycloalkenyl, heterocycloalkyl, or heterocycloalkenyl is substituted with 1 or 2 substituents.

Provided herein as Embodiment 151 is the compound or salt of Embodiment 149 or 150, wherein each substituent independently is halo, C₁₋₃ alkyl, C₁₋₃ haloalkyl, C₀₋₂ alkyleneOH, C₀₋₂ alkyleneC₁₋₃ alkoxy, or C₀₋₂ alkyleneCN.

Provided herein as Embodiment 152 is the compound or salt of Embodiment 151, wherein each substituent independently is F, Cl, OH, OCH₃, OCH₂CH₃, or CN.

Provided herein as Embodiment 153 is the compound or salt of any one of Embodiments 132-134, wherein two non-adjacent R⁶ join together to form a C₁₋₃ alkylene bridge, a C₂₋₃ alkenylene bridge, a C₁₋₃ ether bridge, or a C₁₋₃ thioether bridge.

Provided herein as Embodiment 154 is the compound or salt of Embodiment 153, wherein two non-adjacent R⁶ join together to form —CH₂—, —CH₂CH₂—, —CH₂CH₂CH—, —CH₂—CH═CH— or —CHOCH₂—.

Provided herein as Embodiment 155 is the compound or salt of Embodiment 154, wherein two non-adjacent R⁶ join together to form

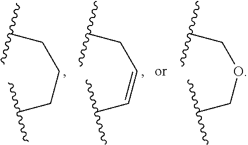

Provided herein as Embodiment 156 is the compound or salt of any one of Embodiments 131-155, wherein Y is N.

Provided herein as Embodiment 157 is the compound or salt of any one of Embodiments 131-155, wherein Y is CH.

Provided herein as Embodiment 158 is the compound or salt of any one of Embodiments 131-155, wherein Y is C-halo, C—CN, C—C₁₋₃ alkyl, C—C₁₋₃ haloalkyl, C—C₀₋₃ alkyleneOH, or C—C₀₋₃ alkylene-C₁₋₄ alkoxy.

Provided herein as Embodiment 150 is the compound or salt of Embodiment 158, wherein Y is C—F, C—Cl, C—CH₃, C—CH₂CH₃, C—CH₂F, C—CHF₂, C—CF₃, C—OH, C—CH₂OH, C—OCH₃, or C—CH₂OCH₃.

Provided herein as Embodiment 160 is the compound or salt of any one of Embodiments 131-159, wherein X is

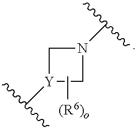

Provided herein as Embodiment 161 is the compound or salt of Embodiment 160, wherein X is or

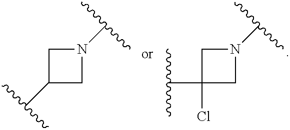 [SMILES: *C1CN(*)C1 ; *N1CC(*)(Cl)C1]

Provided herein as Embodiment 162 is the compound or salt of Embodiment 161, wherein X is

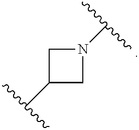 [SMILES: *C1CN(*)C1]

Provided herein as Embodiment 163 is the compound or salt of any one of Embodiments 131-159, wherein X is

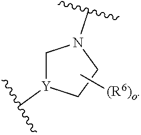

Provided herein as Embodiment 164 is the compound or salt of Embodiment 163, wherein X is

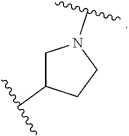 [SMILES: *C1CCN(*)C1]

Provided herein as Embodiment 165 is the compound or salt of any one of Embodiments 131-159, wherein X is

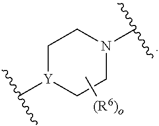

Provided herein as Embodiment 166 is the compound or salt of Embodiment 129, wherein X is

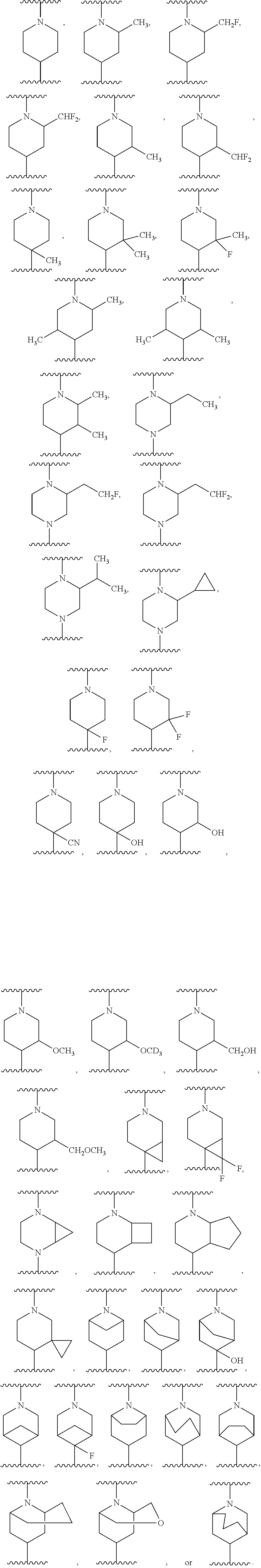

Provided herein as Embodiment 167 is the compound or salt of Embodiment 166, wherein X is

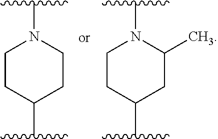

Provided herein as Embodiment 168 is the compound or salt of Embodiment 129, wherein X is

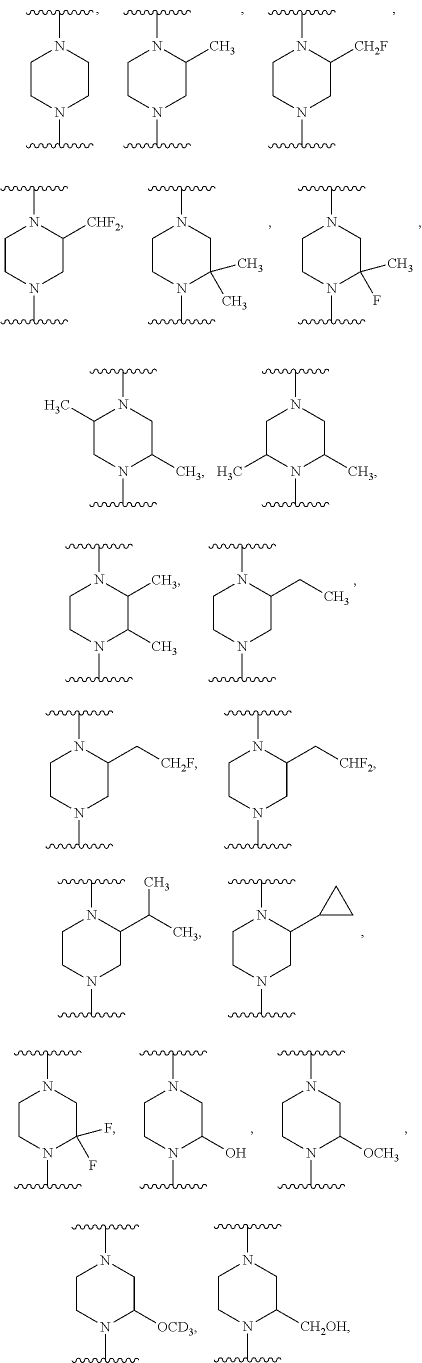

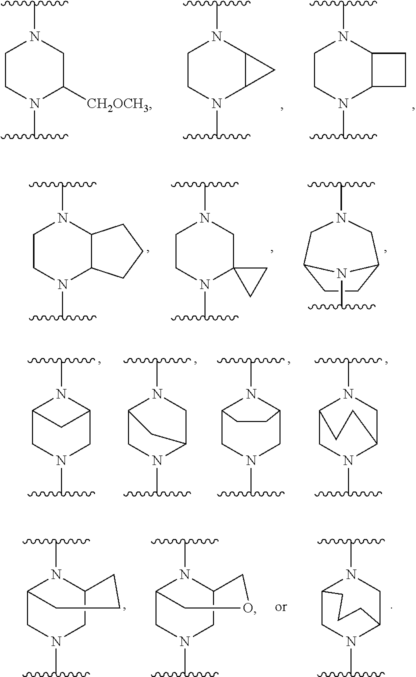

Provided herein as Embodiment 169 is the compound or salt of Embodiment 168, wherein X is

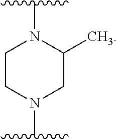 [SMILES: *N1CCN(*)C(C)C1]

Provided herein as Embodiment 170 is the compound or salt of any one of Embodiments 131-159, wherein X is

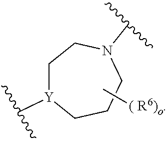

Provided herein as Embodiment 171 is the compound or salt of Embodiment 170, wherein X is

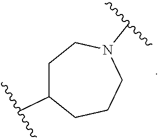 [SMILES: *C1CCCN(*)CC1]

Provided herein as Embodiment 172 is the compound or salt of any one of Embodiments 131-159, wherein X is

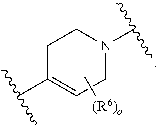

Provided herein as Embodiment 173 is the compound or salt of Embodiment 172, wherein X is

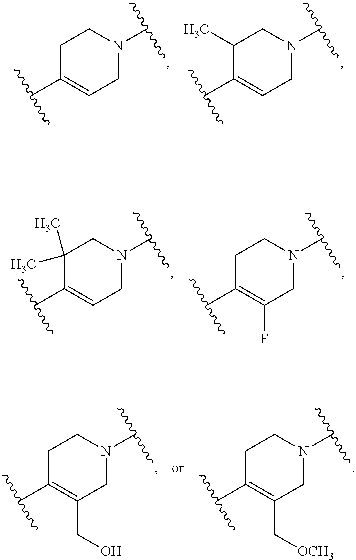

Provided herein as Embodiment 174 is the compound or salt of Embodiment 173, wherein X is

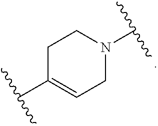 [SMILES: *C1=CCN(*)CC1]

Provided herein as Embodiment 175 is the compound or salt of Embodiment 129, wherein X is

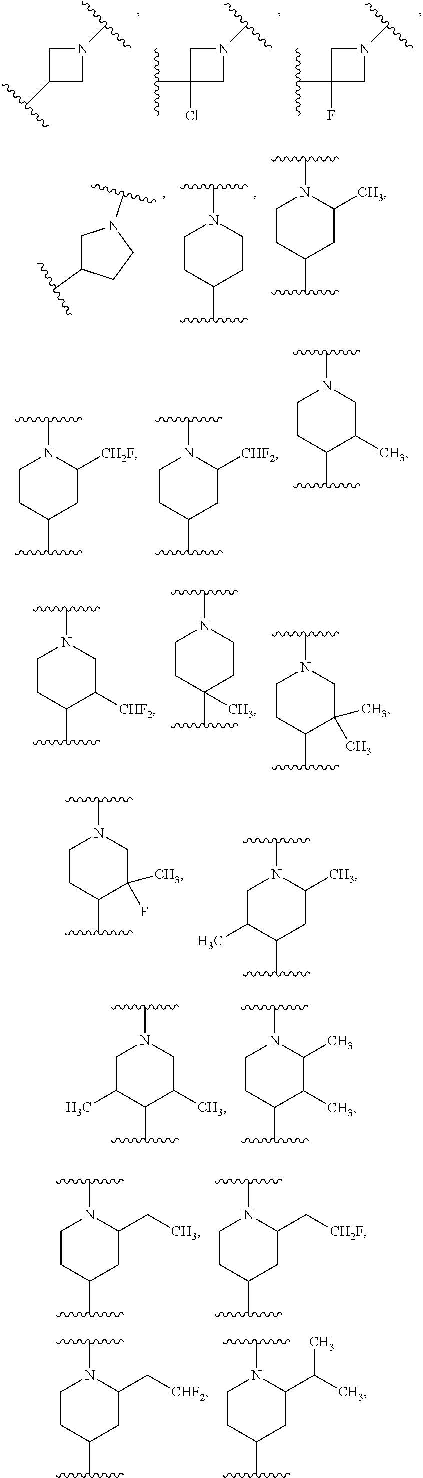

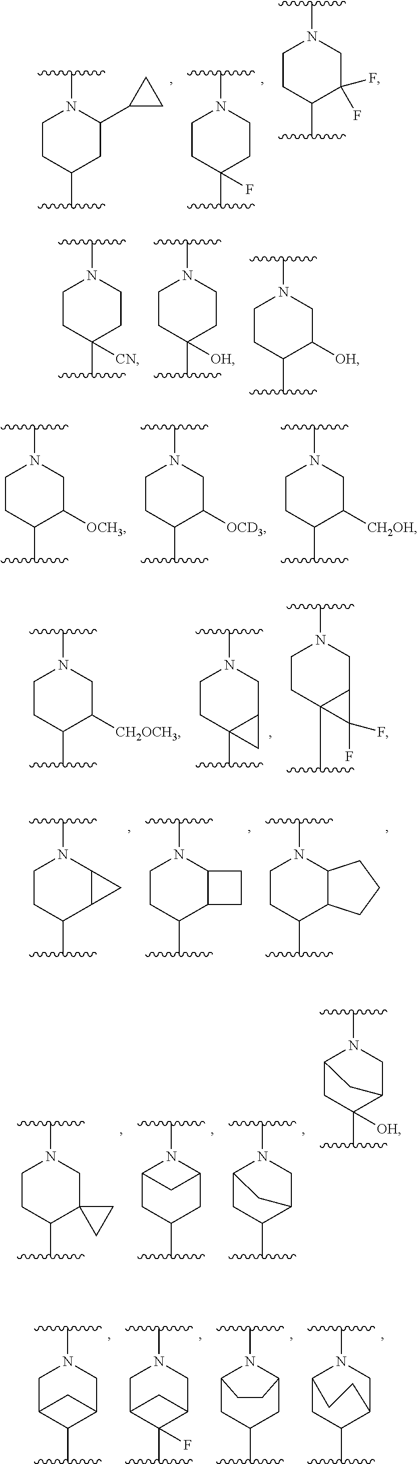

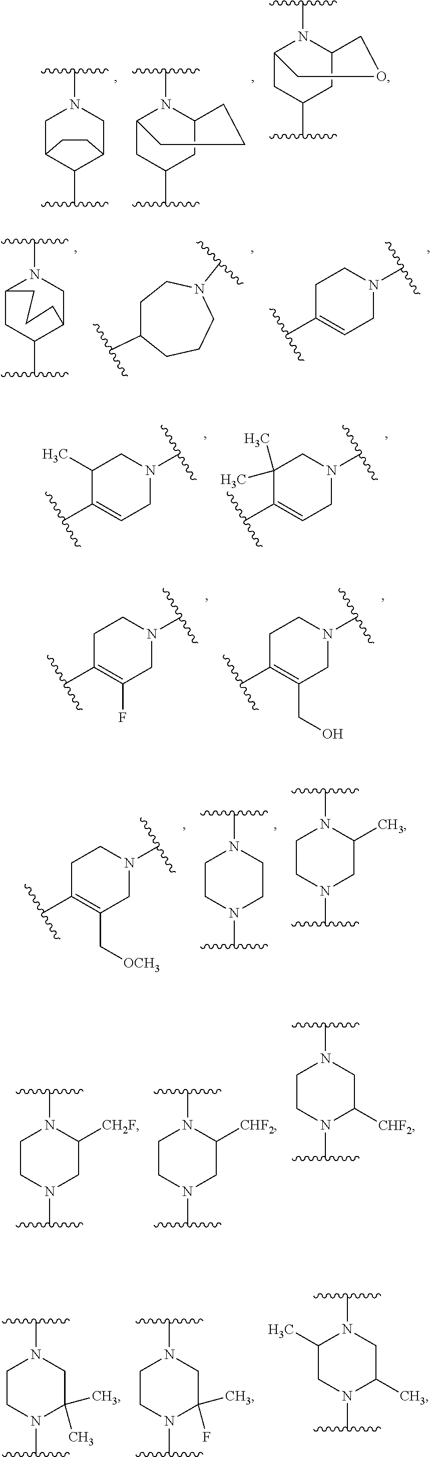

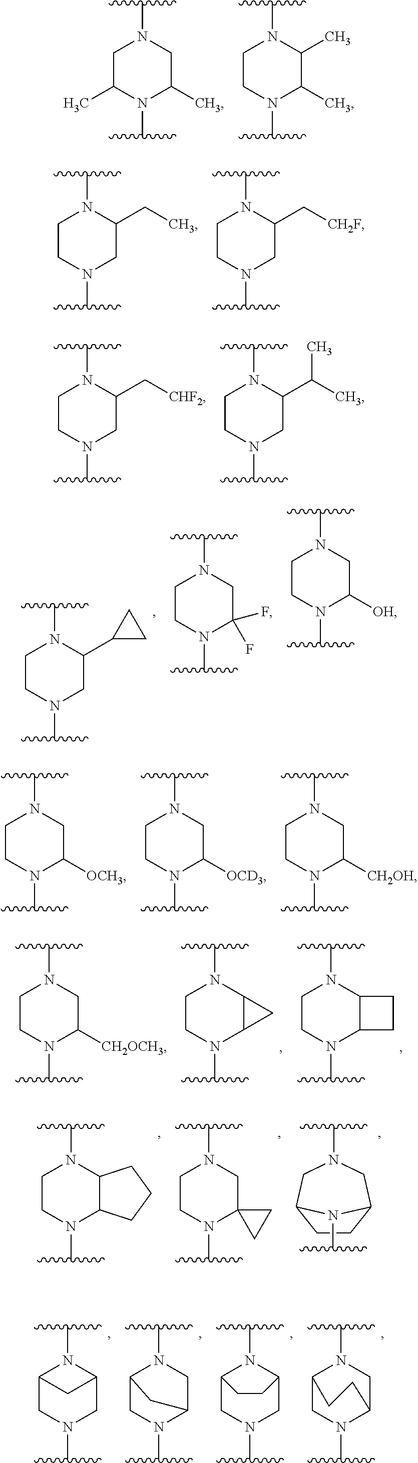

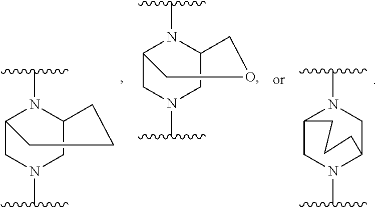 [SMILES: *N1CC2CCCC(C1)N2* ; *N1CC2COCC(C1)N2* ; *N1CC2CCCC1CN2*]

Provided herein as Embodiment 176 is the compound or salt of Embodiment 175, wherein X is

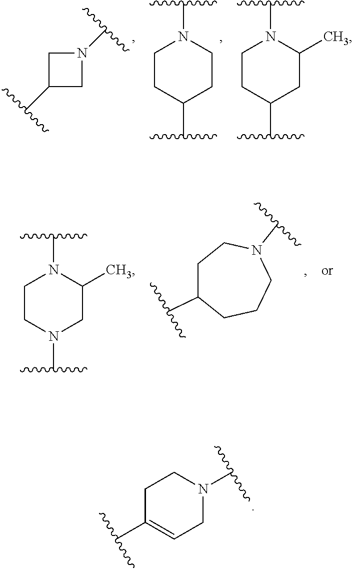

Provided herein as Embodiment 177 is the compound or salt of Embodiment 176, wherein X is

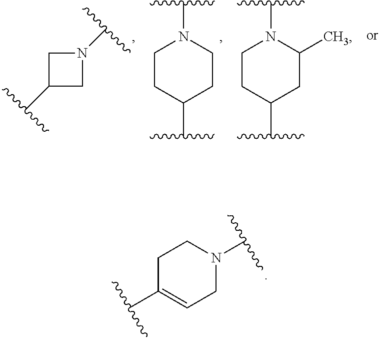

Provided herein as Embodiment 178 is the compound or salt of any one of Embodiments 1-177, wherein Z is phenyl, wherein the phenyl is optionally substituted with 1-4 substituents.

Provided herein as Embodiment 179 is the compound or salt of Embodiment 178, wherein each substituent independently is halo, C₀₋₃ alkyleneCN, C₀₋₃ alkyleneOH, C₀₋₃ alkylene-C₁₋₄ alkoxy, C₀₋₃ alkylene-C₁₋₄ thioalkoxy, or

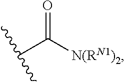

wherein each R^{N1} independently H or CH₃.

Provided herein as Embodiment 180 is the compound or salt of Embodiment 179, wherein each substituent independently is F, Cl, CN, OCH₃, SCH₃, CH₂OH, or

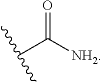 [SMILES: *C(N)=O]

Provided herein as Embodiment 181 is the compound or salt of Embodiment 180, wherein Z is

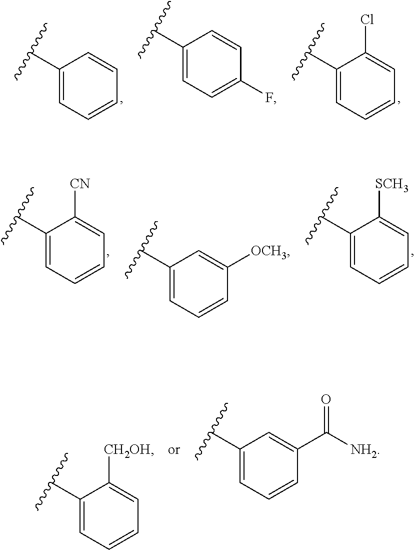

Provided herein as Embodiment 182 is the compound or salt of any one of Embodiments 1-177, wherein Z is heteroaryl comprising 5 or 6 total ring atoms and 1-3 heteroatoms selected from N, O, and S, wherein the heteroaryl is optionally substituted with 1 or more substituents.

Provided herein as Embodiment 183 is the compound or salt of Embodiment 182, wherein the heteroaryl is pyrrolyl, furanyl, thiophenyl, pyrazolyl, imidazol, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl.

Provided herein as Embodiment 184 is the compound or salt of Embodiment 183, wherein the heteroaryl is pyrazolyl, imidazolyl, thiazolyl, isothiazolyl, oxazolyl, isoxazolyl, or triazolyl.

Provided herein as Embodiment 185 is the compound or salt of Embodiment 183, wherein the heteroaryl is pyridyl, pyridazinyl, pyrimidinyl, or pyrazinyl.

Provided herein as Embodiment 186 is the compound or salt of Embodiment 183, wherein the heteroaryl is pyrazolyl, thiazolyl, pyridyl, or pyridazinyl.

Provided herein as Embodiment 187 is the compound or salt of Embodiment 186, wherein the heteroaryl is pyrazolyl or pyridyl.

Provided herein as Embodiment 188 is the compound or salt of Embodiment 187, wherein the heteroaryl is pyrazolyl.

Provided herein as Embodiment 189 is the compound or salt of Embodiment 187, wherein the heteroaryl is pyridyl.

Provided herein as Embodiment 190 is the compound or salt of any one of Embodiments 182-189, wherein the heteroaryl is unsubstituted.

Provided herein as Embodiment 191 is the compound or salt of any one of Embodiments 182-189, wherein the heteroaryl is substituted with 1-4 substituents.

Provided herein as Embodiment 192 is the compound or salt of Embodiment 191, wherein each substituent independently is halo, CN, C₁₋₆ alkyl, C₁₋₆ haloalkyl, C₂₋₆ alkenyl, C₂₋₆ haloalkenyl, C₀₋₆ alkylene-OH, C₀₋₆ alkylene-C₁₋₃ alkoxy, C₀₋₆ alkylene-N(R^{N1})₂, C₀₋₂ alkylene-cycloalkyl having 3-6 total ring atoms. C₀₋₂ alkylene-heterocycloalkyl having 3-6 total ring atoms and 1-3 heteroatoms selected from N, O, and S. or C₀₋₂ alkylene-phenyl; wherein each of the C₁₋₆ alkyl, C₂₋₆ alkenyl, C₀₋₆ alkylene-C₁₋₃ alkoxy, cycloalkyl, heterocycloalkyl, and phenyl substituents is optionally substituted with 1 or more further substituents, and each R^{N1} independently is H or C₁₋₃ alkyl.

Provided herein as Embodiment 193 is the compound or salt of Embodiment 192, wherein each of the C₁₋₆ alkyl, C₂₋₆ alkenyl, C₀₋₆ alkylene-C₁₋₃ alkoxy, C₃₋₇ cycloalkyl, heterocycloalkyl, and phenyl substituents is optionally substituted with 1-3 further substituents.

Provided herein as Embodiment 194 is the compound or salt of Embodiment 193, wherein each of the C₁₋₆ alkyl, C₂₋₆ alkenyl, C₀₋₆ alkylene-C₁₋₃ alkoxy, C₃₋₇ cycloalkyl, heterocycloalkyl, and phenyl substituents is optionally substituted with 1 or 2 further substituents.

Provided herein as Embodiment 195 is the compound or salt of Embodiment 194, wherein each of the C₁₋₆ alkyl, C₂₋₆ alkenyl, C₀₋₆ alkylene-C₁₋₃ alkoxy, C₃₋₇ cycloalkyl, heterocycloalkyl, and phenyl substituents is optionally substituted with 1 further substituent.

Provided herein as Embodiment 196 is the compound or salt of Embodiment 192, wherein the C₁₋₄ alkyl is CH₃, CH₂CH₃, CH₂CH₂CH₃, or CH(CH₃)₂, and each of the foregoing is optionally substituted with 1 or more further substituents.

Provided herein as Embodiment 197 is the compound or salt of Embodiment 196, wherein the C₁₋₆ alkyl is CH₃, and the CH₃ is optionally substituted with 1 or more further substituents.

Provided herein as Embodiment 198 is the compound or salt of Embodiment 192, wherein the C₂₋₆ alkenyl is CH═CH₂, CH₂CH═CH₂, or CH═CHCH₃, and each of the foregoing independently is optionally substituted with 1 or more further substituents.

Provided herein as Embodiment 199 is the compound or salt of Embodiment 192, wherein the C₀₋₆ alkylene-C₁₋₃ alkoxy is OCH₃, CH₂OCH₃, CH₂CH₂OCH₃, CH, CH₂OCH₂CH₃, CH₂CH₂CH₂OCH₃, CH(CH₃)₂OCH₃, CH(CH₃)CH₂OCH₃, CH(OCH₃)CH₂OCH₃, CH(CH₃)(OCH₃)CH₂OCH₃, C(CH₃)₂OCH₃, C(CH₃)₂CH₂OCH₃, CH₂CH(CH₃)OCH₃, CH₂(CH₃)(OCH₃)OCH₃, CH₂C(CH₃)₂OCH₃, or CH₂C(CH₃)₂OCH₃, and each of the foregoing is optionally substituted with 1 or more further substituents.

Provided herein as Embodiment 200 is the compound or salt of Embodiment 199, wherein the C₀₋₆ alkylene-C₁₋₃ alkoxy, is CH(CH₃)OCH₃ or CH₂CH₂OCH₃, and each of the foregoing is optionally substituted with 1 or more further substituents.

Provided herein as Embodiment 201 is the compound or salt of Embodiment 192, wherein the cycloalkyl having 3-6 total ring atoms is cyclopropyl, cyclobutyl, cyclopentyl, or cyclohexyl, and each of the foregoing is optionally substituted with 1 or more further substituents.

Provided herein as Embodiment 202 is the compound or salt of Embodiment 201, wherein the cycloalkyl having 3-6 total ring atoms is cyclopropyl or cyclobutyl, and each of the foregoing is optionally substituted with 1 or more further substituents.

Provided herein as Embodiment 203 is the compound or salt of Embodiment 192, wherein the heterocycloalkyl having 3-6 total ring atoms and 1-3 heteroatoms selected from N, O, and S is azetidinyl, pyrrolidinyl, piperidinyl, pyrazolidinyl, oxetanyl, tetrahydrofuranyl, tetrahydropyranyl, isoxazolidinyl, or morpholinyl, and each of the foregoing is optionally substituted with 1 or more further substituents.

Provided herein as Embodiment 204 is the compound or salt of Embodiment 203, wherein the heterocycloalkyl having 3-6 total ring atoms and 1-3 heteroatoms selected from N, O, and S is azetidinyl, oxetanyl, tetrahydrofuranyl, tetrahydropyranyl, or morpholinyl, and each of the foregoing is optionally substituted with 1 or more further substituents.

Provided herein as Embodiment 205 is the compound or salt of Embodiment 204, wherein the heterocycloalkyl having 3-6 total ring atoms and 1-3 heteroatoms selected from N, O, and S is azetidinyl or oxetanyl, and each of the foregoing is optionally substituted with 1 or more further substituents.

Provided herein as Embodiment 206 is the compound or salt of Embodiment 192, wherein at least one substituent is Br, Cl, F, or CN.

Provided herein as Embodiment 207 is the compound or salt of Embodiment 192, wherein the C₁₋₆ haloalkyl is CF₃, CHF₂, CH₂F, CH₂CHF₂, CH₂CH₂F, CH(CH₂F)₂, CH(CH₃)CH₂F, or CH(CH₃)CHF₂.

Provided herein as Embodiment 208 is the compound or salt of Embodiment 192, wherein the C₁₋₆ haloalkenyl is C(═CH₂)CH₂F.

Provided herein as Embodiment 209 is the compound or salt of Embodiment 192, wherein C₀₋₆ alkylene-OH is OH, CH₂OH, CH₂CH₂OH, CH(CH₄)CH₂OH, C(CH₃)₂OH, C(CH₃)₂CH₂OH, or CH₂C(CH₃)₂OH.

Provided herein as Embodiment 210 is the compound or salt of Embodiment 192, wherein the C₀₋₃ alkylene-N(R^{N1})₂, is NH₂, CH₂NH₂, CH₂NHCH₃, CH₂N(CH₃)₂, CH₂CH₂NH₂, CH₂CH₂NHCH₃, or CH₂CH₂N(CH₃).

Provided herein as Embodiment 211 is the compound or salt of any one of Embodiments 192-205, wherein each further substituent independently is D, halo, C₁₋₃ alkyl, C₁₋₃ haloalkyl, C₁₋₂ alkyleneOH, C₁₋₂ alkylene-C₁₋₃ alkoxy, C₁₋₃ deuterated alkoxy, N(R^{N1})₂, (C═O)C₁₋₃ alkyl, cycloalkyl having 3-5 total ring atoms, or heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S; or two adjacent further substituents, together with the atoms to which they are attached, form fused-cycloalkyl having 3-5 total ring atoms or fused-heterocycloalkyl having 3-5 total ring atoms and 1 or 2 heteroatoms selected from N, O, and S; wherein each of the foregoing cycloalkyl and heterocycloalkyl groups is optionally substituted with halo or C₁₋₃ alkyl and each R^{N1} independently is H or C₁₋₃ alkyl.

Provided herein as Embodiment 212 is the compound or salt of Embodiment 211, wherein each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, azetidinyl, spiro-oxetanyl or spiro-azetidinyl; wherein each of the foregoing oxetanyl, azetidinyl, spiro-oxetanyl, and spiro-azetidinyl is optionally substituted with F, CH₃, or a combination thereof.

Provided herein as Embodiment 213 is the compound or salt of Embodiment 212, wherein each further substituent independently is D, Br, Cl, F, OH, CH₃, CF₃, CF₂H, CFH₂, OCH₃, CH₂OCH₃, OCD₃, N(CH₃)₂, (C═O)CH₃,

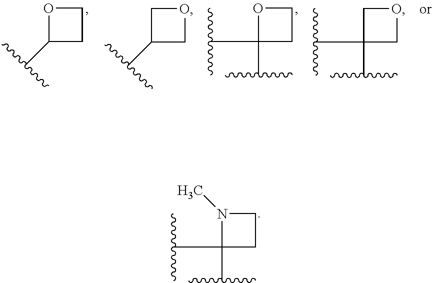

Provided herein as Embodiment 214 is the compound or salt of Embodiment 213, wherein each further substituent independently is D, CH₃, OCH₃, OCD₃, N(CH₃)₂,

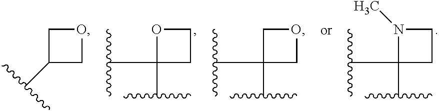

Provided herein as Embodiment 215 is the compound or salt of Embodiment 191 or 192, wherein each of the 1-4 substituents independently is Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH(CH₃CH₃, CH₂CH₂CH₂OCH₃, CH CH₂CH₃OCD₃, CH(CH₃)OCH₃, CH(CH₃)CH₂OCH₃, CH(OCH)CH₂OCH₃, CH(CH)(OCH₃)CH₂OCH₃, CH(CH₂F)(CH₃)CH₂OCD₃, CH(CH₃)CH₂OCD₃, C(CH₃)₂OCH₃, C(CH₃)₂CH₂OCH₃, C(CH)₂CH₂OCD₃, CH₂CH(CH₃)OCH₃, CH₂(CH₃)(OCH₃)OCH₃, CH₂CH(CH₃)OCD₃, CH₂C(CH₃)₂OCH₂C(CH₃)₂OCD₃, NH₂, CH₂NH₂, CH₂NHCH₃, CH₂N(CH₃)₂, CH₂CH₂NH₃, CH₂CH₂NHCH₃, CH₂CH₂N(CH₃)₂,

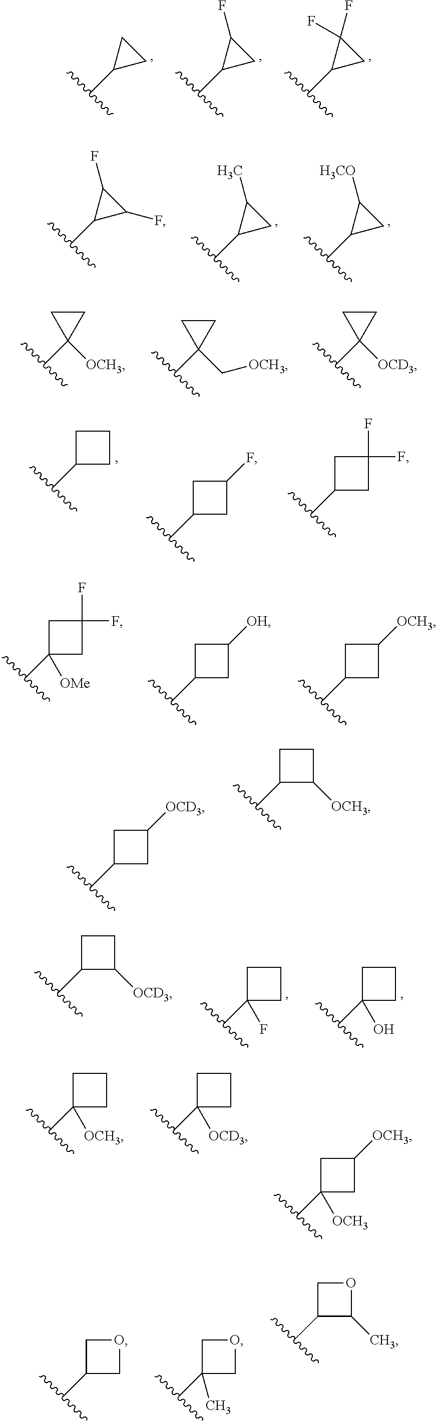

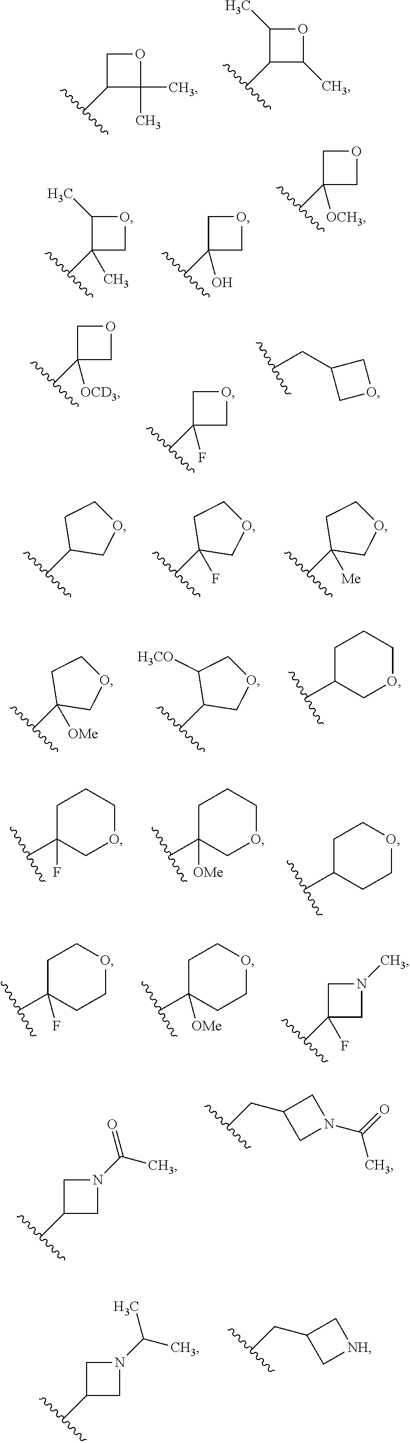

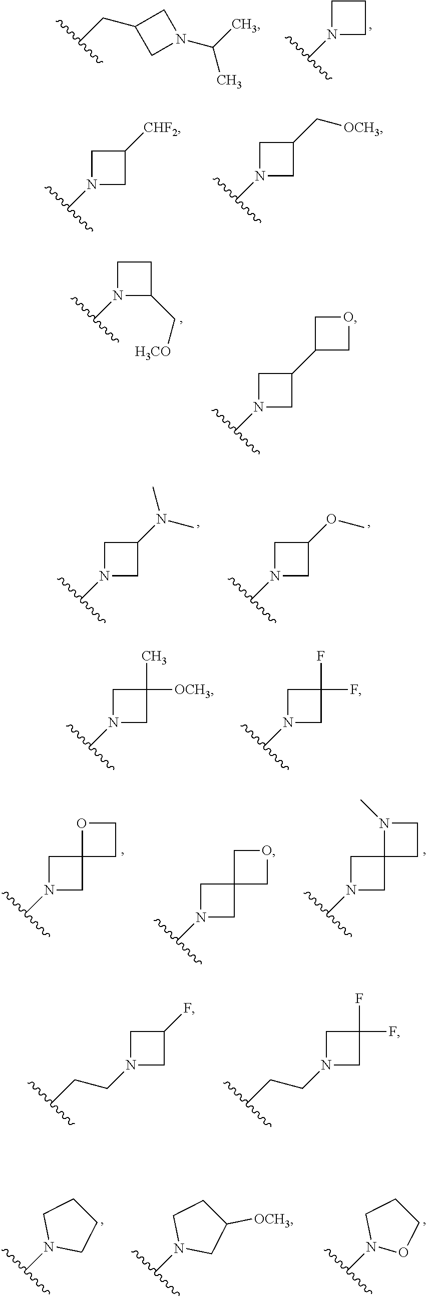

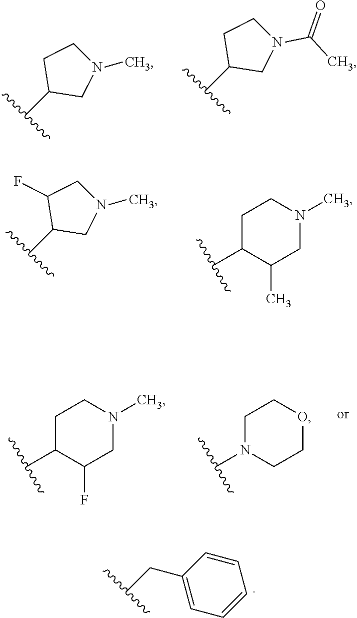

Provided herein as Embodiment 216 is the compound or salt of Embodiment 215, wherein each of the 1-4 substituents independently is CH₃, CH(CH₃)₂, C(CH₃)₂OH, CH₂OCD₃, CH₂CH₂OCH₃, CF₂CH₂OCH₃, CHOCH₂OCD₃, CH₂CH₂CH₂OCH₂, CH₂CH(CH)OCH₃, CH₂C(CH₃)₂OCH₃,

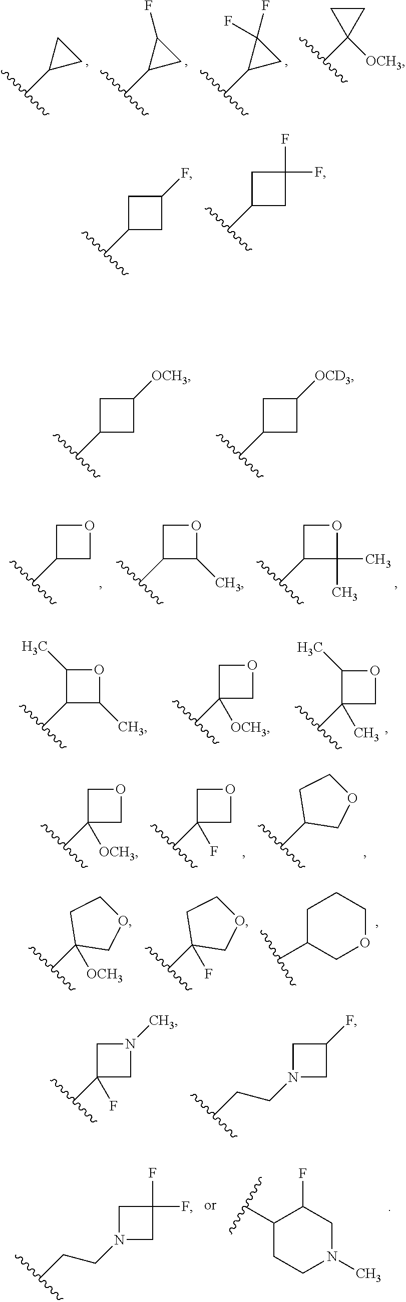

Provided herein as Embodiment 217 is the compound or salt of Embodiment 215, wherein each of the 1-4 substituents independently is CH₃, C(CH₃)₂CH₂OH, CH₂CH₂OCH₃, CH₂CH₂OCD₃, CH(CH₃)OCH₃,

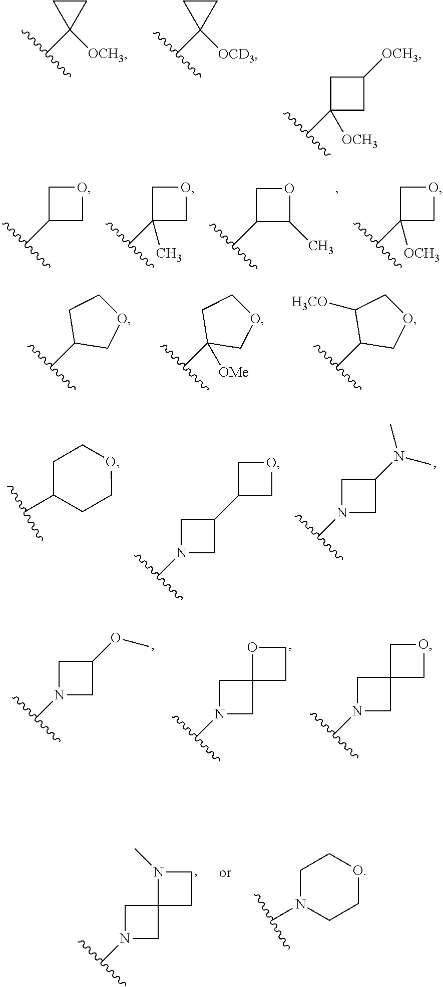

Provided herein as Embodiment 218 is the compound or salt of Embodiment 217, wherein each of the 1-4 substituents independently is CH₃, CH₂CH₂OCH₃,

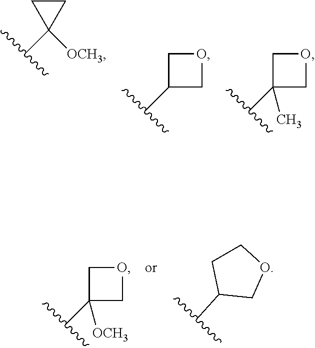

Provided herein as Embodiment 219 is the compound or salt of any one of Embodiments 1-177, wherein Z is

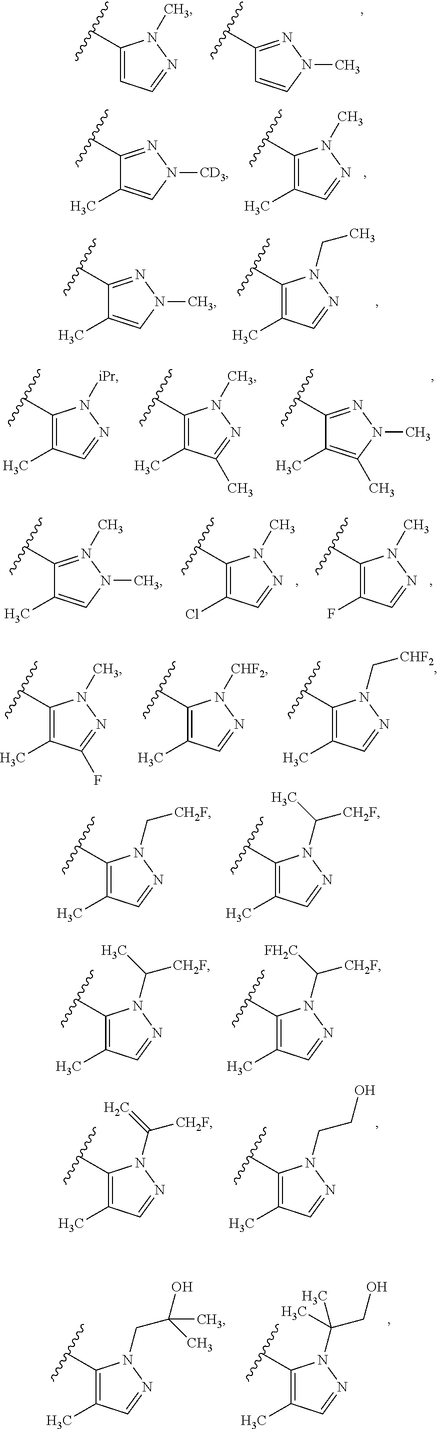

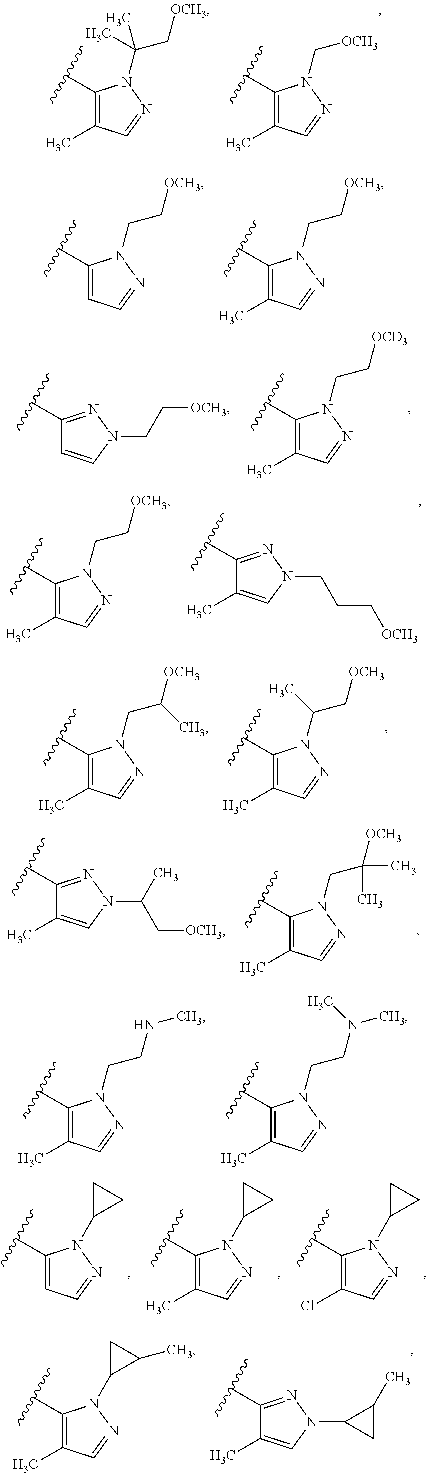

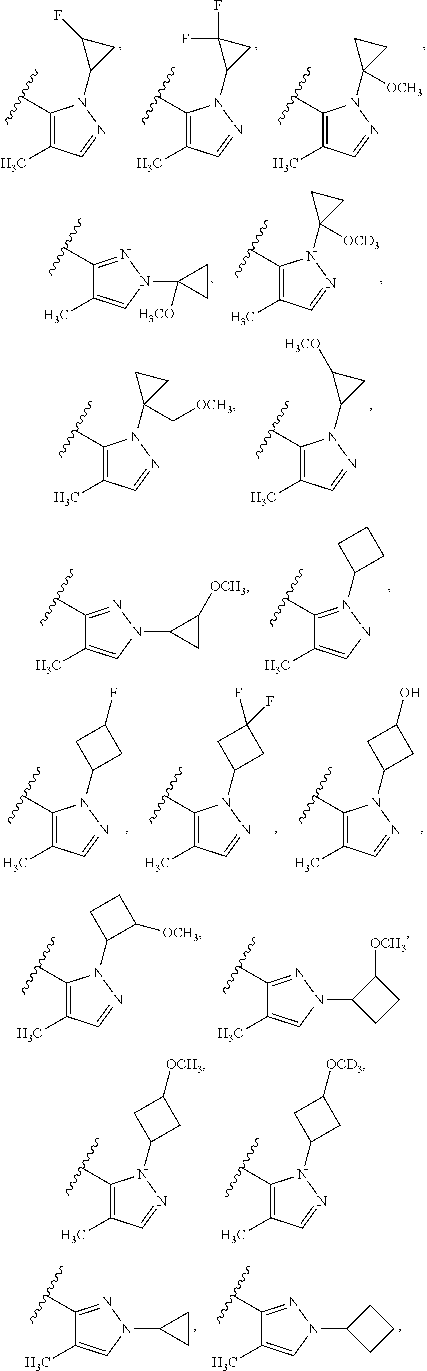

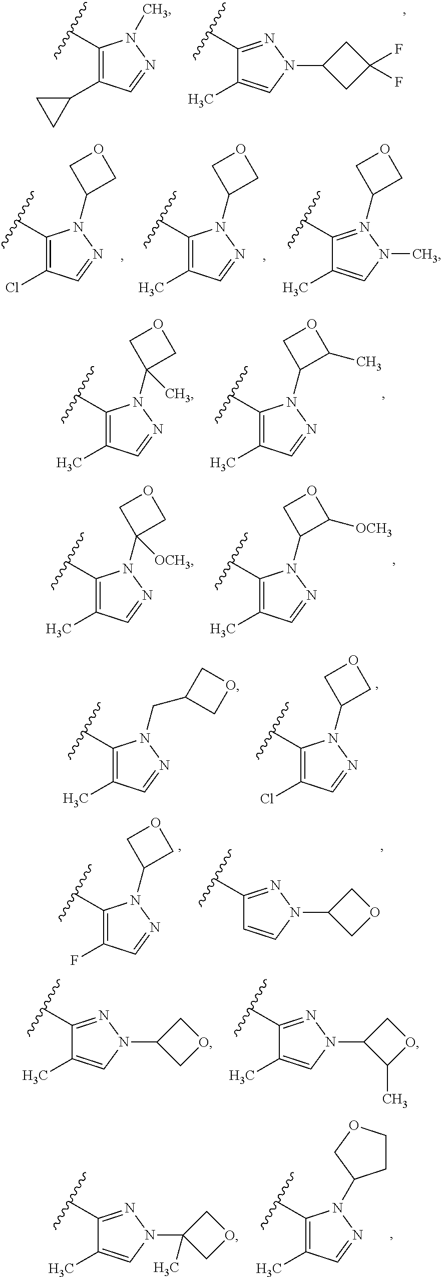

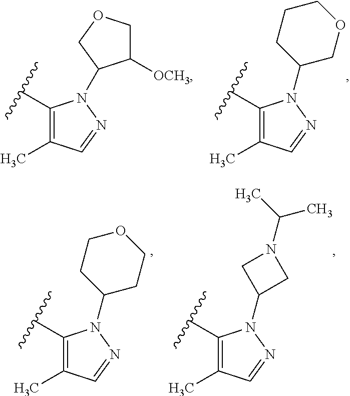

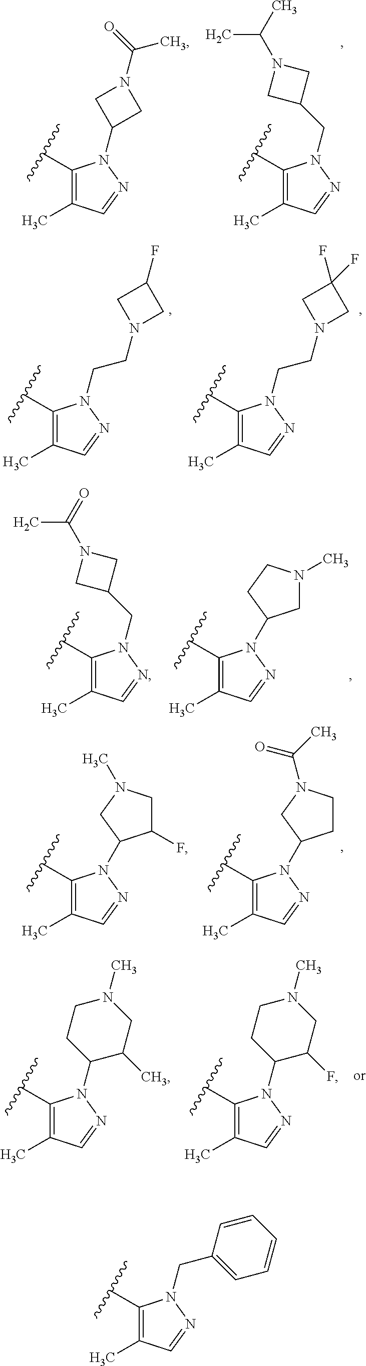

Provided herein as Embodiment 220 is the compound or salt of Embodiment 219, wherein Z is

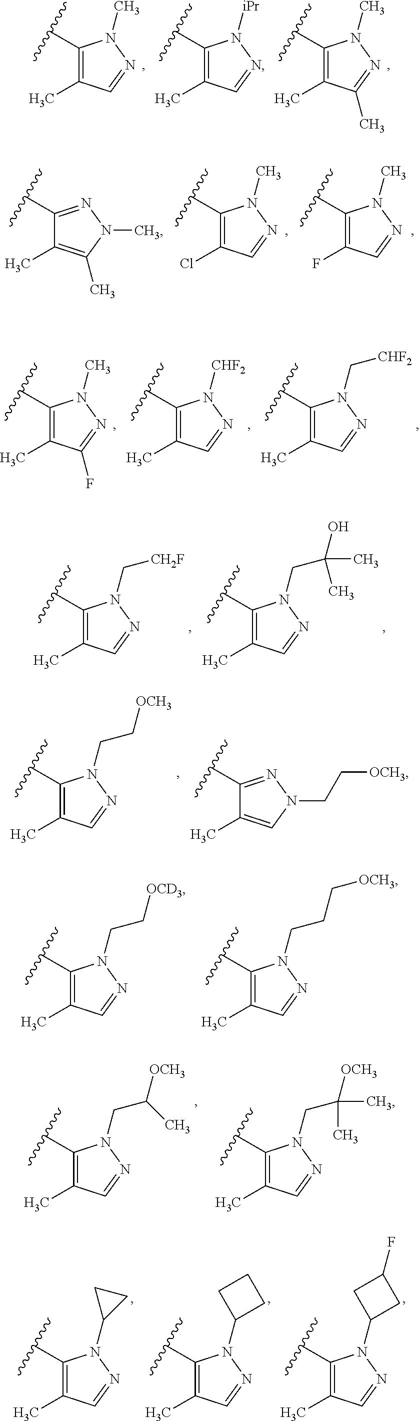

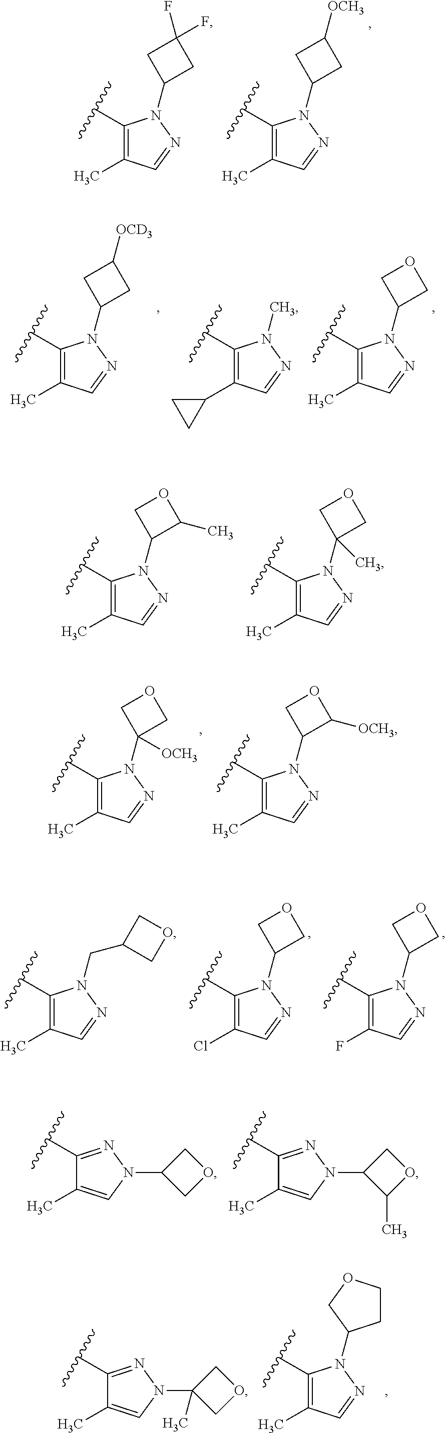

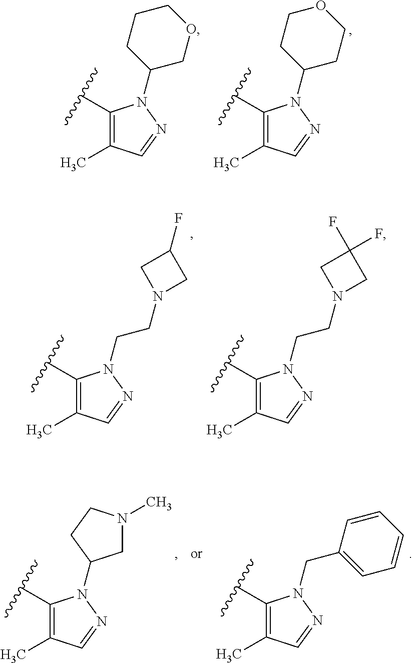 [SMILES: *c1c(C)cnn1C1CCCOC1 ; *c1c(C)cnn1C1CCOCC1 ; *c1c(C)cnn1CCN1CC(F)C1 ; *c1c(C)cnn1CCN1CC(F)(F)C1 ; *c1c(C)cnn1C1CCN(C)C1 ; *c1c(C)cnn1Cc1ccccc1]

Provided herein as Embodiment 221 is the compound or salt of Embodiment 220, wherein Z is

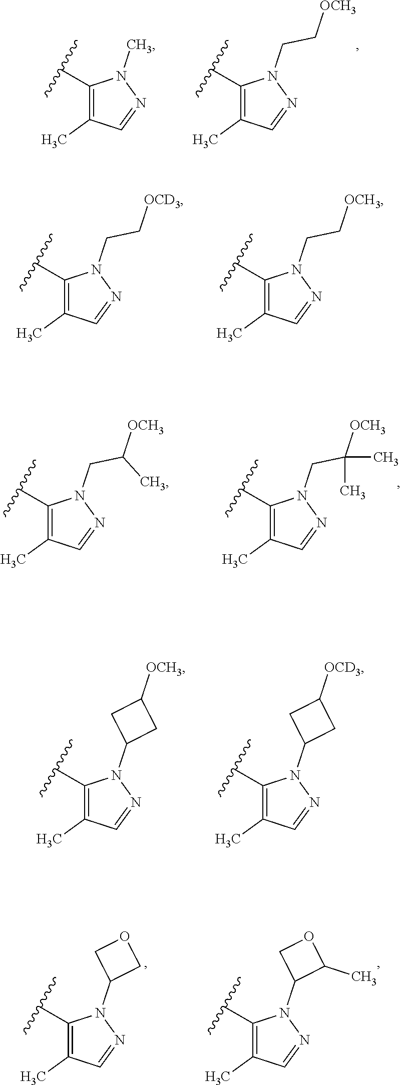

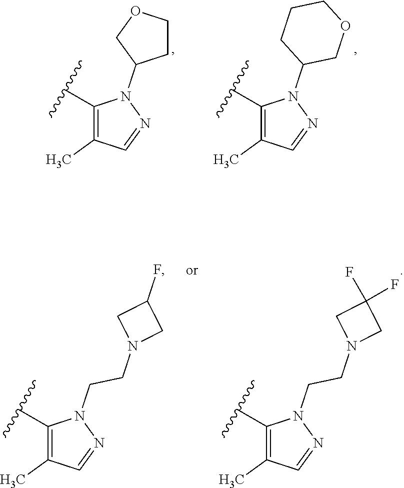 [SMILES: *c1c(C)cnn1C1CCOC1 ; *c1c(C)cnn1C1CCCOC1 ; *c1c(C)cnn1CCN1CC(F)C1 ; *c1c(C)cnn1CCN1CC(F)(F)C1]

Provided herein as Embodiment 222 is the compound or salt of Embodiment 220, wherein Z is

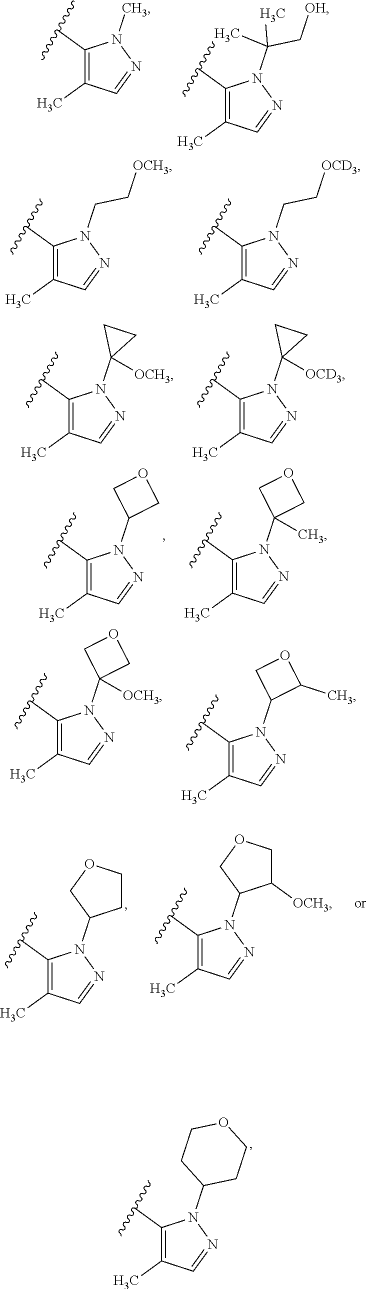

Provided herein as Embodiment 223 is the comp und or salt of any one of Embodiments 1-177 wherein Z is

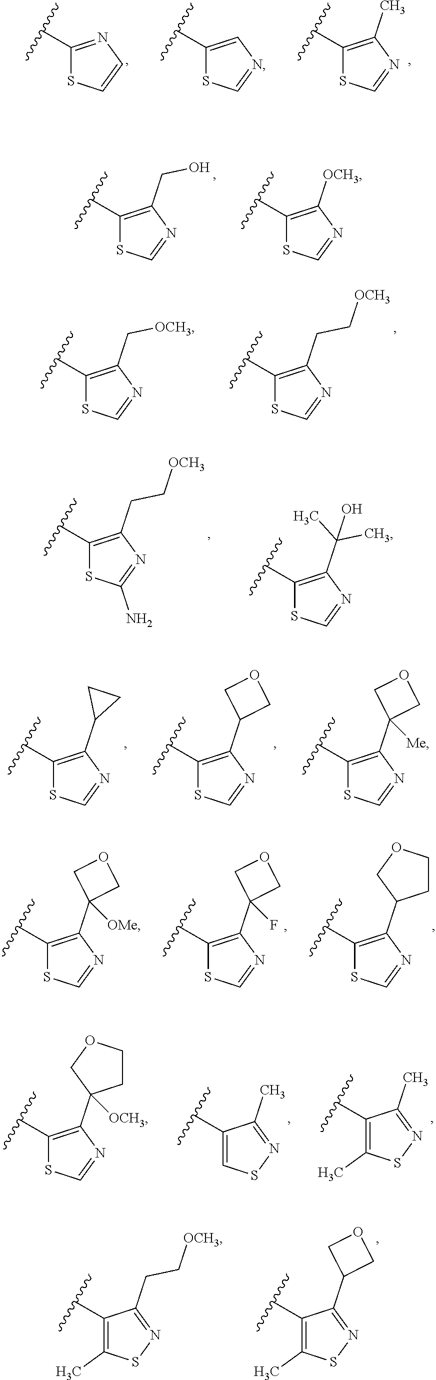

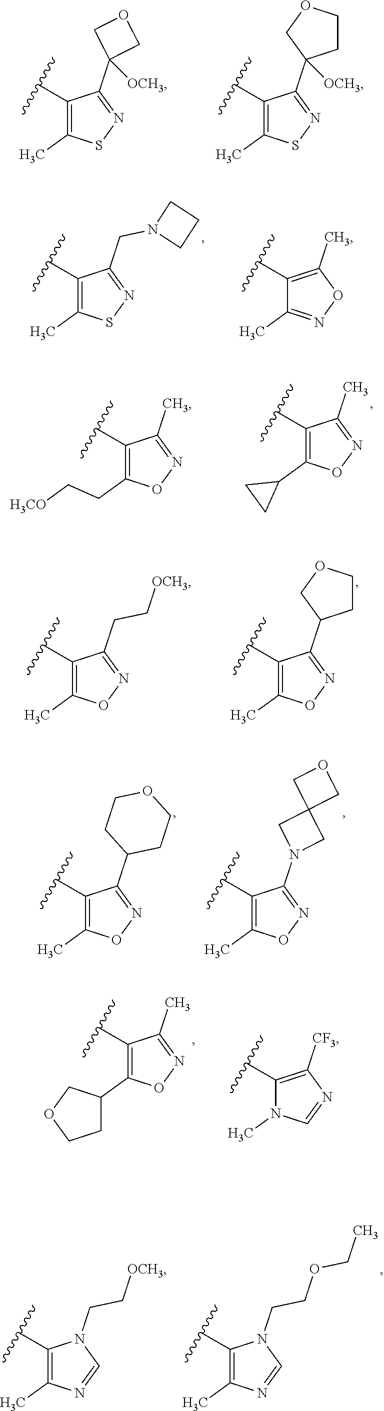

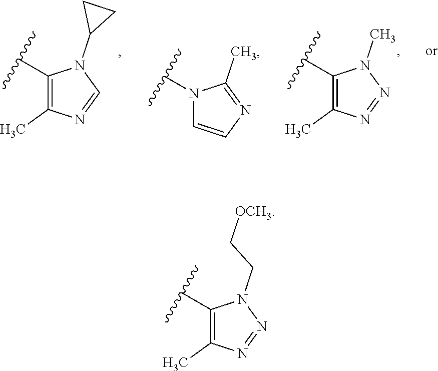

Provided herein as Embodiment 224 is the compound or salt of Embodiment 223, wherein Z, is

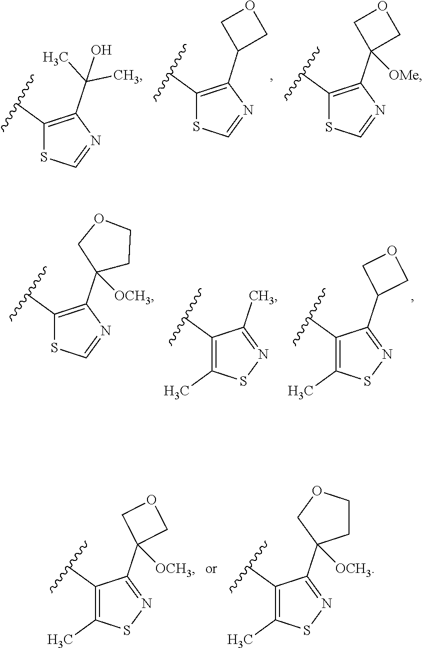

Provided herein as Embodiment 225 is the compound or salt of any one of Embodiments 1-177, wherein Z is

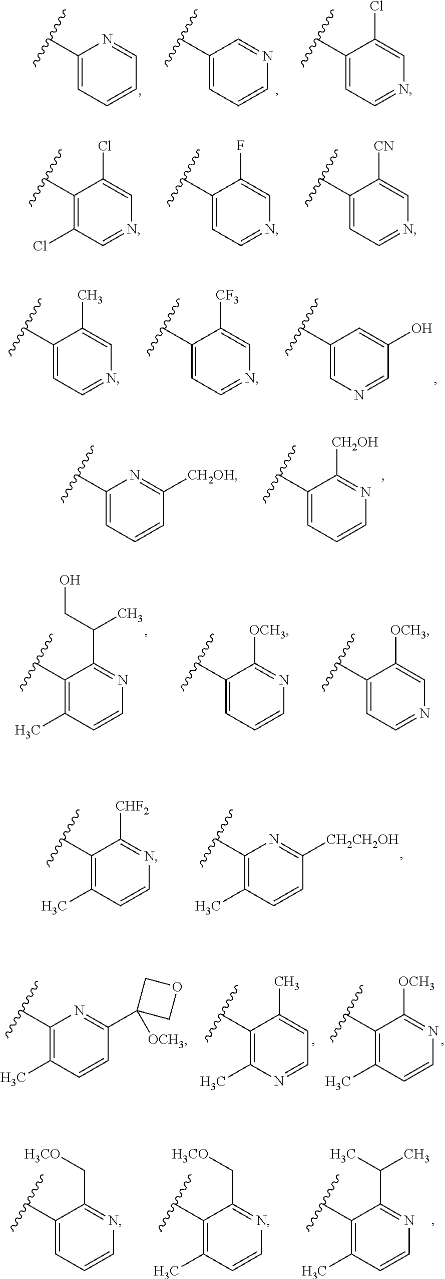

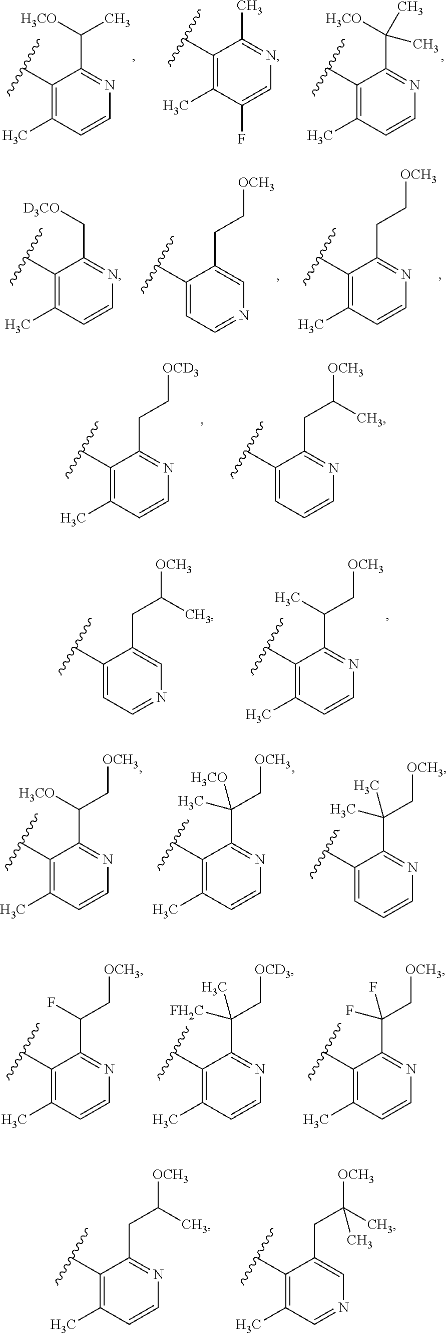

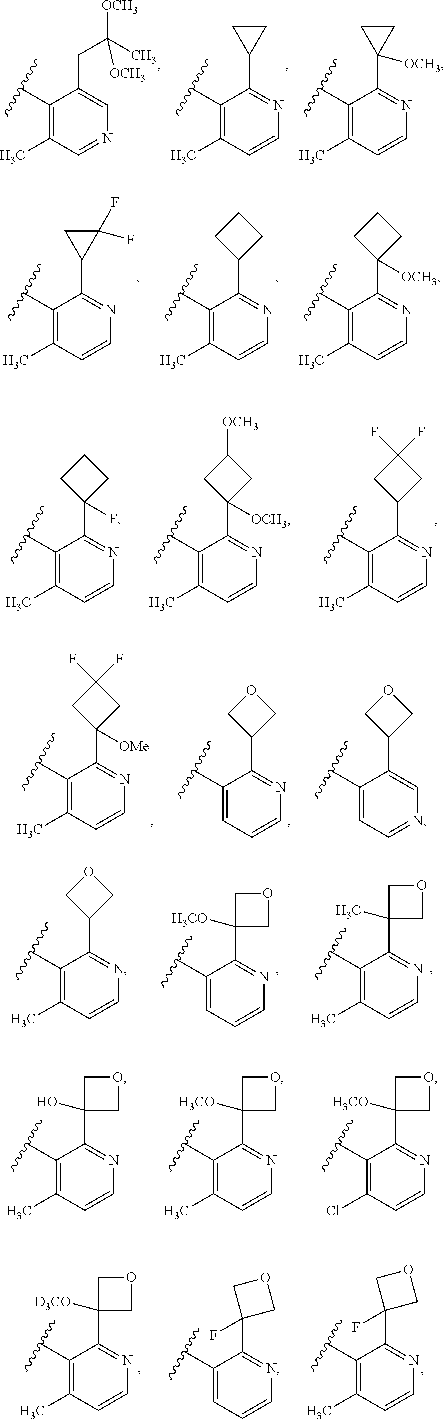

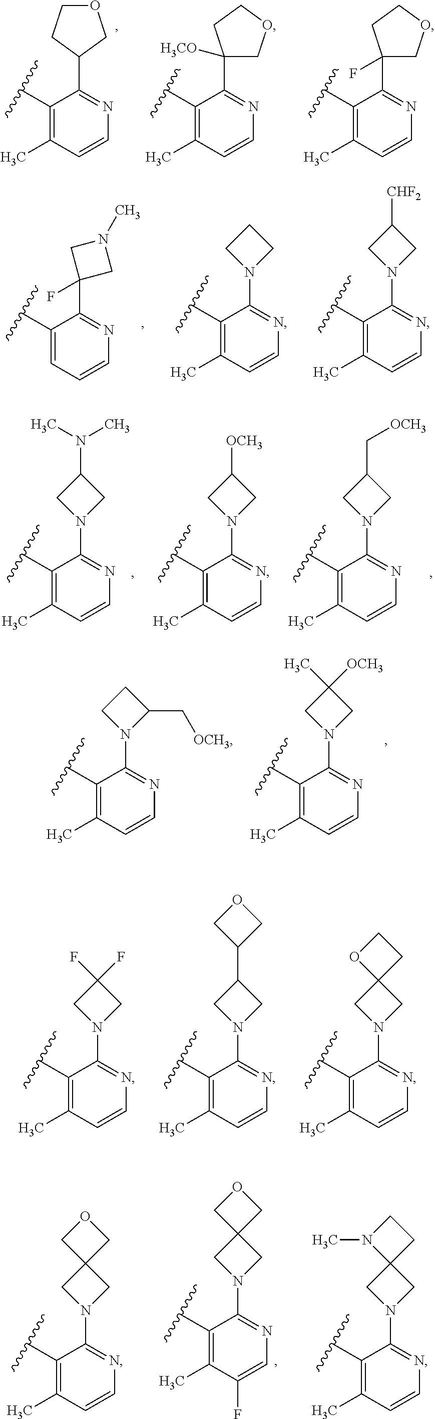

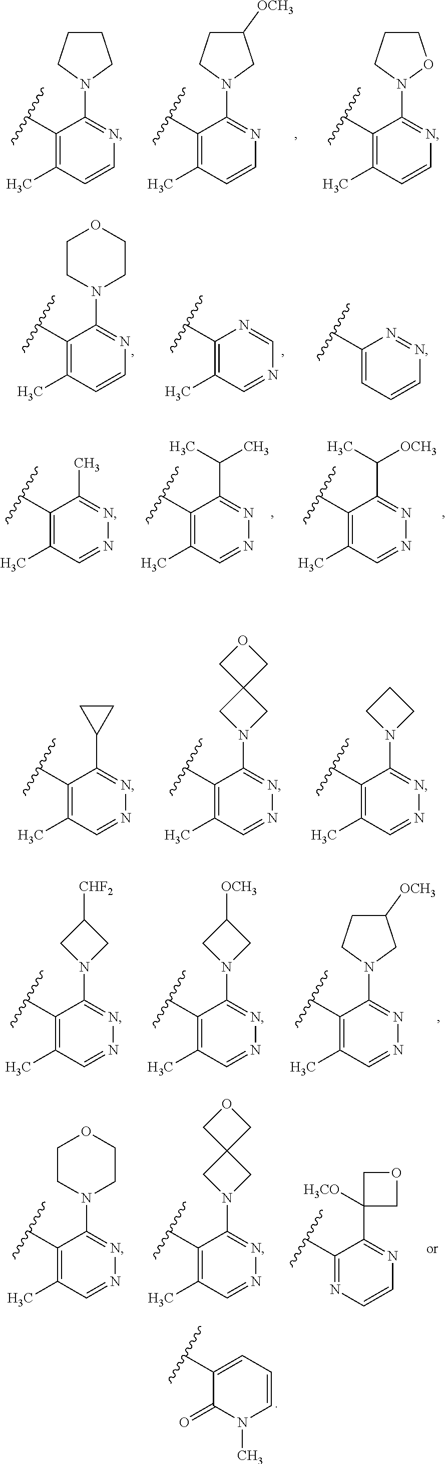

Provided herein as Embodiment 226 is the compound or salt of Embodiment 225, wherein Z is

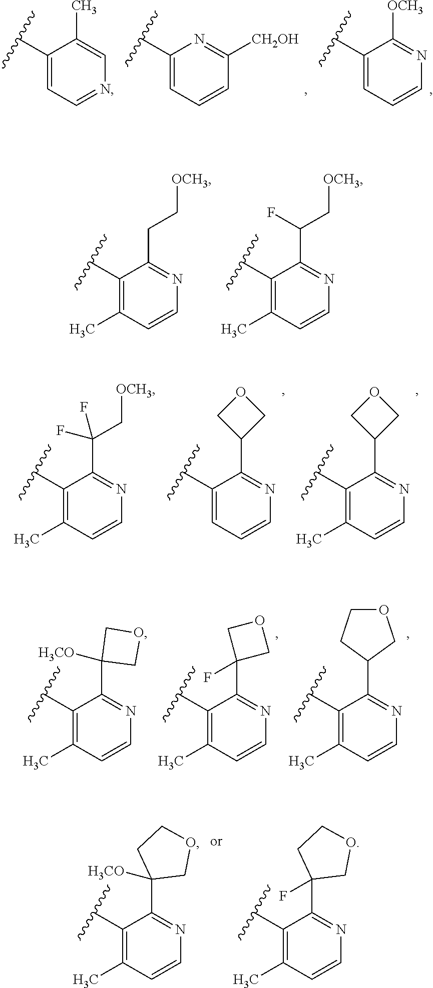

Provided herein as Embodiment 227 is the compound or salt of Embodiment 225, wherein Z is

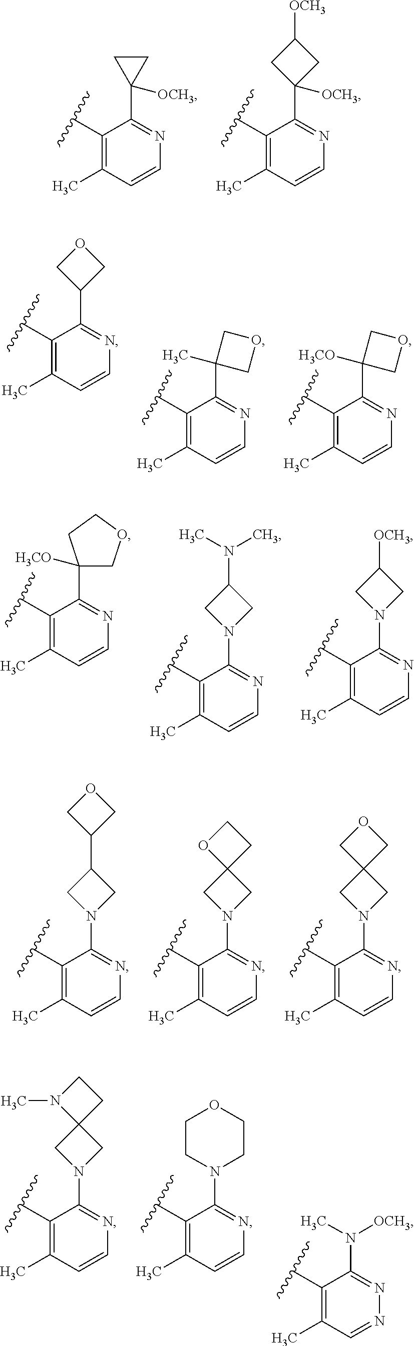

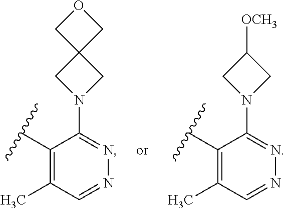 [SMILES: *c1c(C)cnnc1N1CC2(COC2)C1 ; "or" ; *c1c(C)cnnc1N1CC(OC)C1]

Provided herein as Embodiment 228 is the compound or salt of any one of Embodiments 1-177, wherein Z is

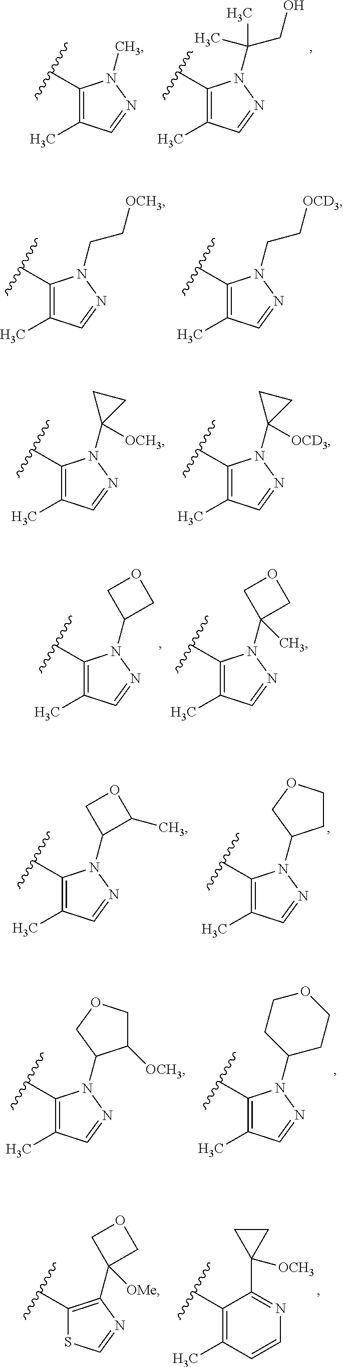

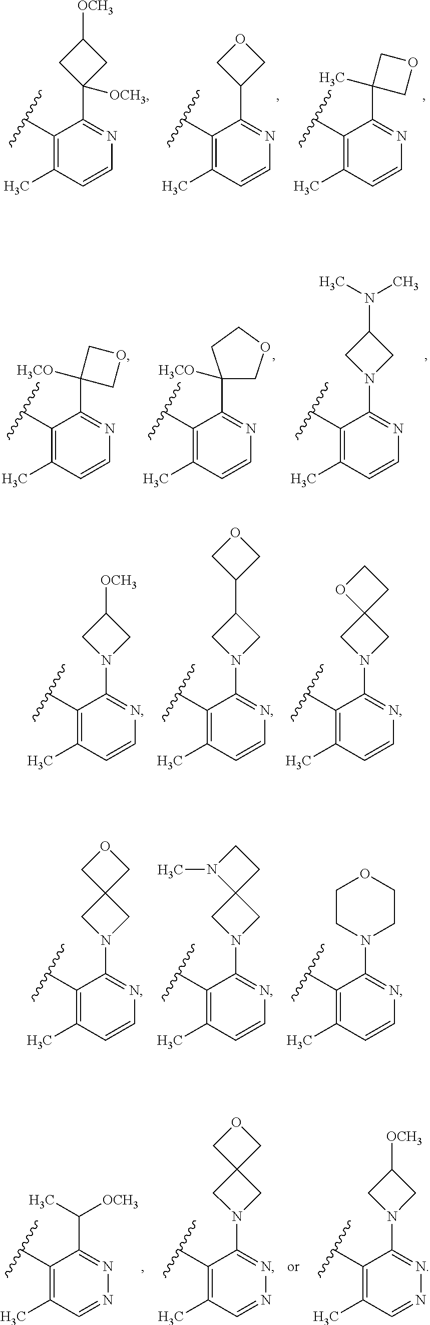

Provided herein as Embodiment 229 is the compound or salt of Embodiments 228, wherein Z is

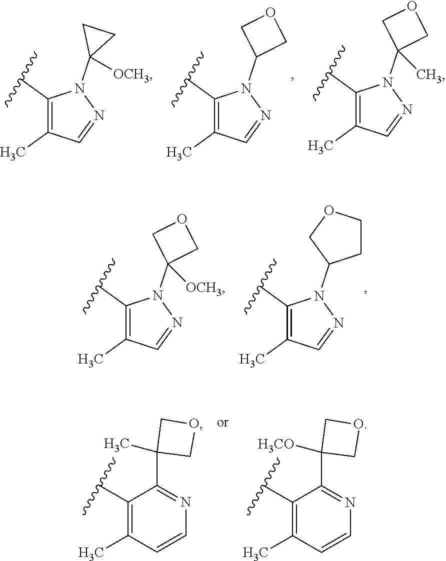

Provided herein as Embodiment 230 is the compound or salt of any one of Embodiments 1-177, wherein Z is a bicyclic ring comprising a heteroaryl ring having 5 or 6 total ring atoms and 1-3 heteroatoms selected from N, O, and S fused to a ring having 5 or 6 total ring atoms and 0, 1, or 2 heteroatoms selected from N, O, and S; wherein the bicyclic ring is optionally substituted with 1-4 substituents.

Provided herein as Embodiment 231 is the compound or salt of Embodiment 230, wherein the heteroaryl ring is pyridyl, pyridazinyl, pyrimidinyl, or pyrazinyl; and the fused ring has 5 total atoms and 1 oxygen atom in the fused ring, 5 total atoms and 1 nitrogen atom in the fused ring, 6 total atoms and 1 nitrogen or oxygen atom in the ring, or 6 total atoms, 1 oxygen atom, and 1 nitrogen atom in the fused ring.

Provided herein as Embodiment 232 is the compound or salt of Embodiment 230 or 231, wherein the bicyclic ring is substituted with halo, CN, C₁₋₄ alkyl, C₁₋₆ haloalkyl, C₀₋₆ alkylene-OH, or C₀₋₆ alkylene-C₁₋₃ alkoxy, or any combination of the foregoing.

Provided herein as Embodiment 233 is the compound or salt of any one of Embodiments 230-232, wherein Z is

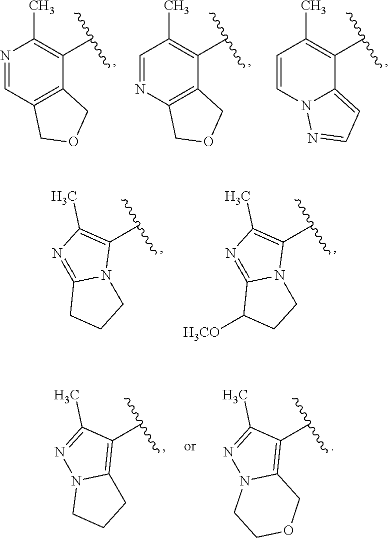

Provided herein as Embodiment 234 is the compound or salt of Embodiment 1, wherein:

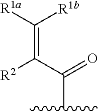

is

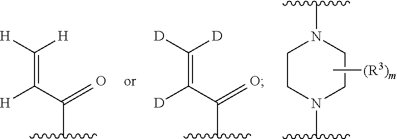

is

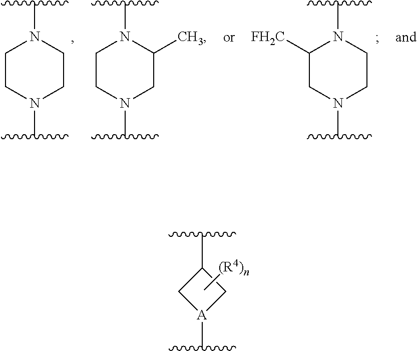

is

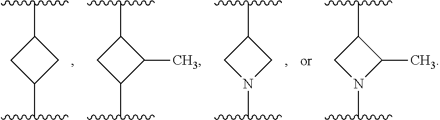

Provided herein as Embodiment 235 is the compound of Embodiment 234, wherein

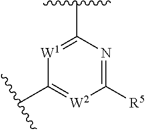

is

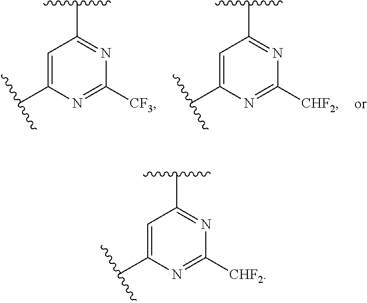 [SMILES: *c1cc(*)nc(C(F)(F)F)n1 ; *c1cc(*)nc(C(F)F)n1 ; *c1cc(*)nc(C(F)F)n1]

Provided herein as Embodiment 236 is the compound or salt of Embodiment 234 or 235, wherein X is

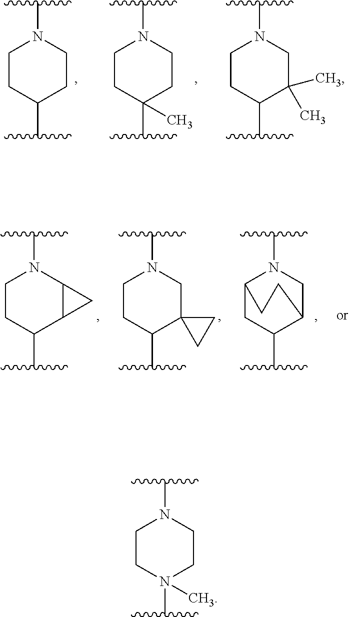

Provided herein as Embodiment 237 is the compound or salt of any one of Embodiments 234-236, wherein Z is

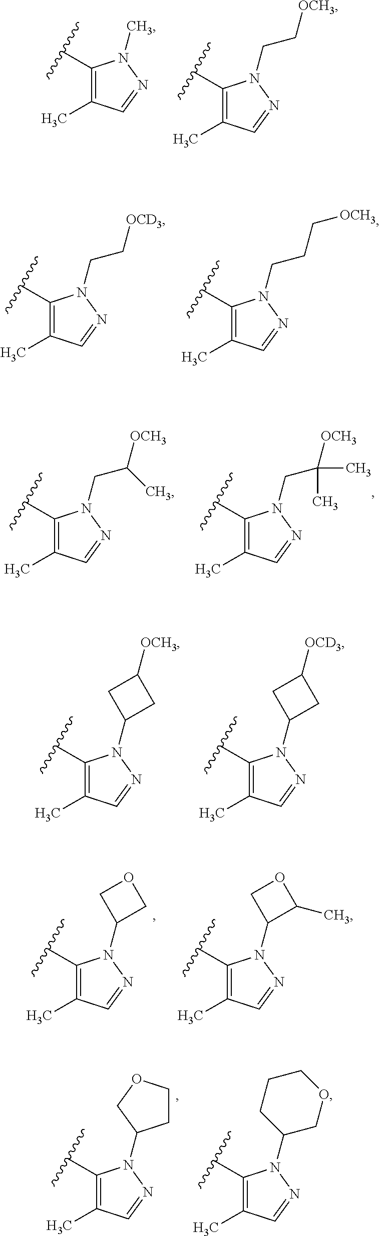

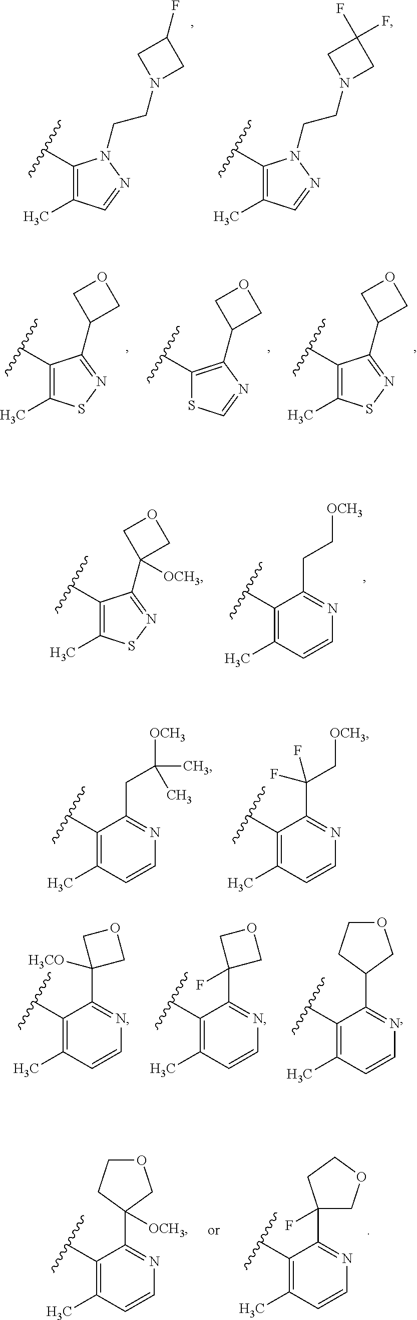

Provided herein as Embodiment 238 is the compound or salt of Embodiment 237, wherein Z is

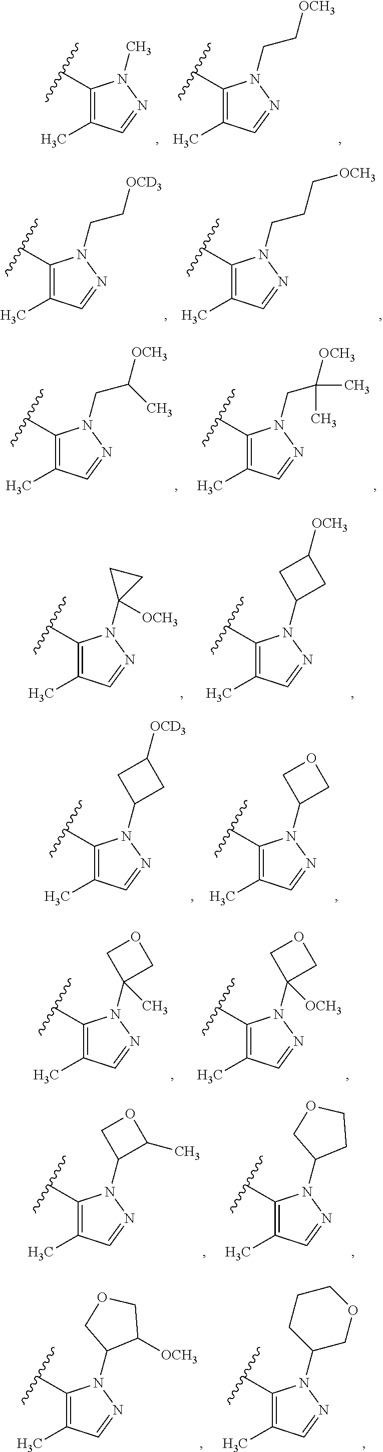

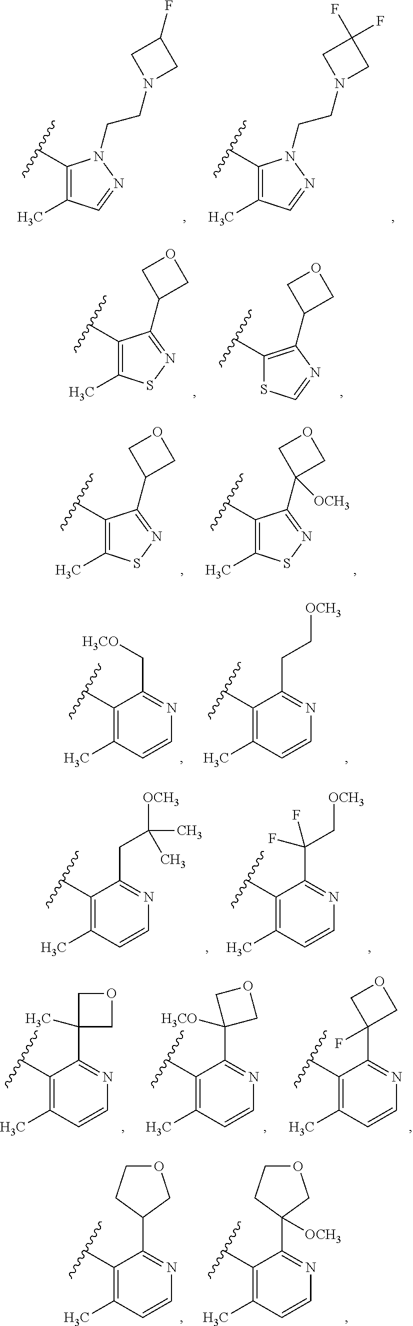

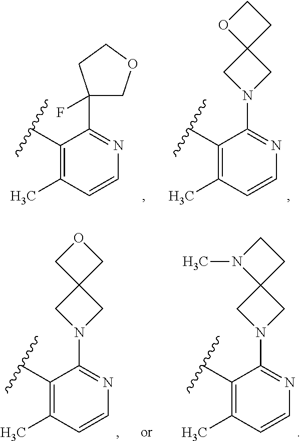

Provided herein as Embodiment 239 is the compound or salt of Embodiment 238, wherein Z is

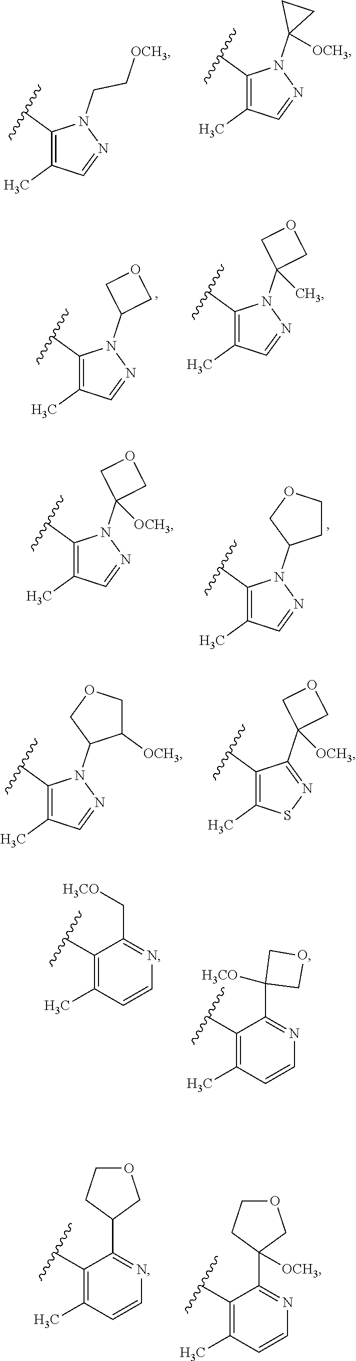

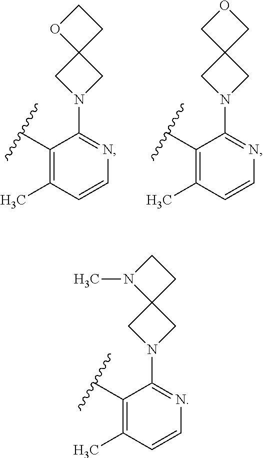 [SMILES: *c1c(C)ccnc1N1CC2(CCO2)C1 ; *c1c(C)ccnc1N1CC2(COC2)C1 ; *c1c(C)ccnc1N1CC2(CCN2C)C1]

Provided herein as Embodiment 240 is the compound or salt of Embodiment 239, wherein Z is

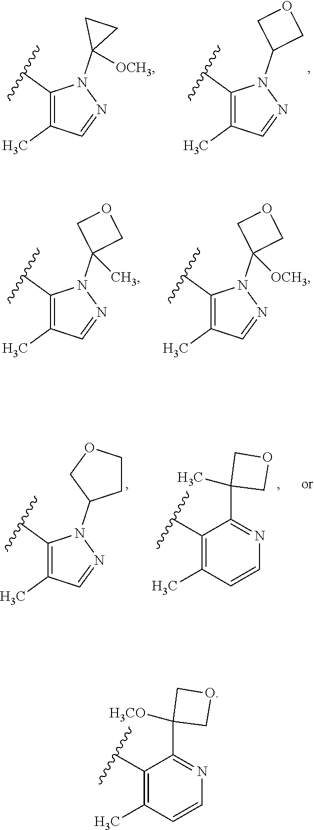

Provided herein as Embodiment 241 is the compound or salt of any one of Embodiments 234-236, wherein Z is

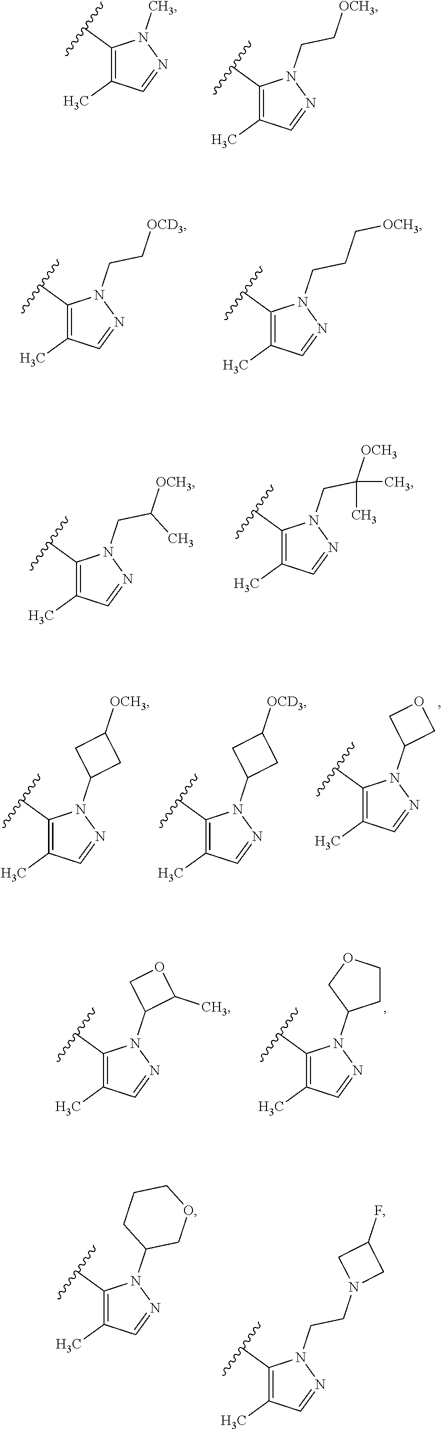

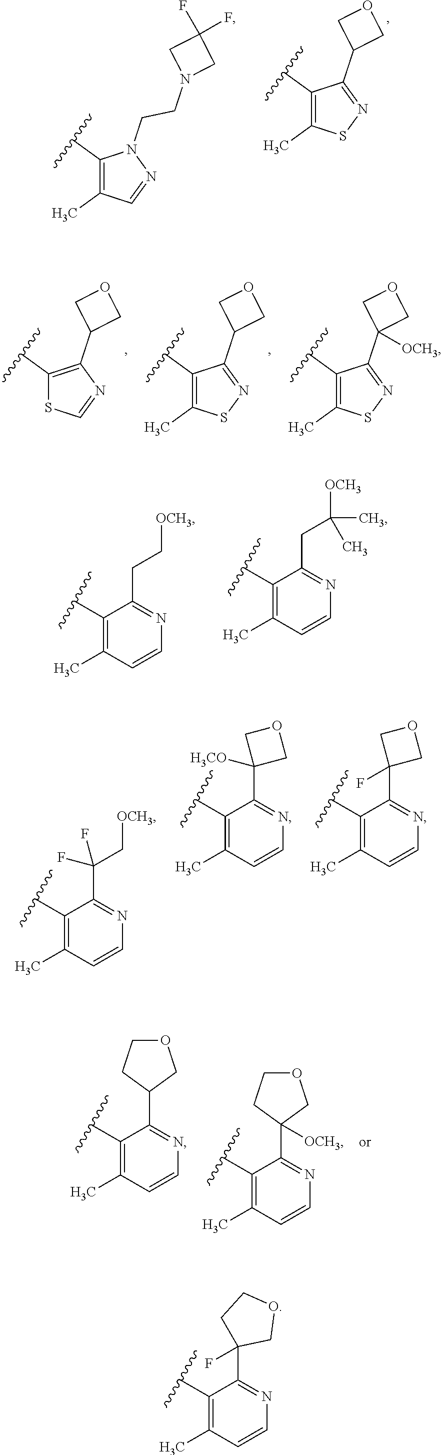

Provided herein as Embodiment 242 is the compound or salt of Embodiment 1, wherein the compound is a compound of Formula (I′):

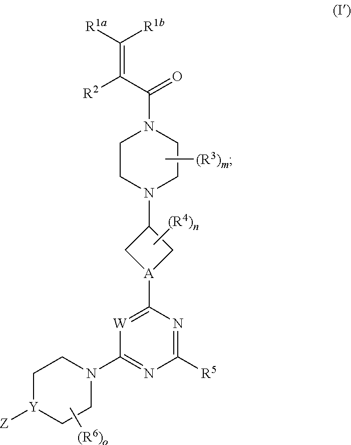

### Formula (IA):

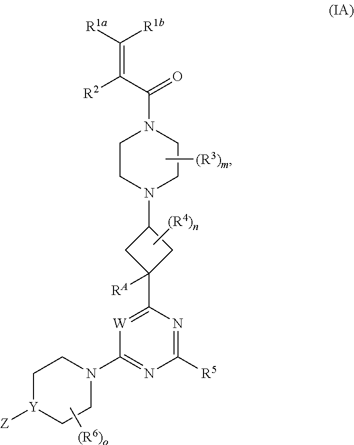

wherein R^{A} is H, halo, CN, C₁₋₃ alkyl, C₁₋₃ haloalkyl, C₀₋₃ alkyleneOH, or C₀₋₃ alkylene-C₁₋₄ alkoxy; Formula (IB):

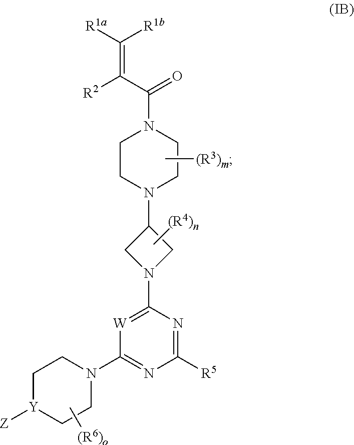

### Formula (IC):

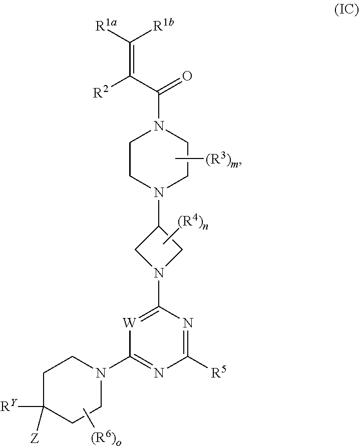

wherein R^{Y} is H, halo, CN, C₁₋₃ alkyl, C₁₋₃ haloalkyl, C₀₋₃ alkyleneOH, or C₀₋₃ alkylene-C₁₋₄ alkoxy; Formula (ID):

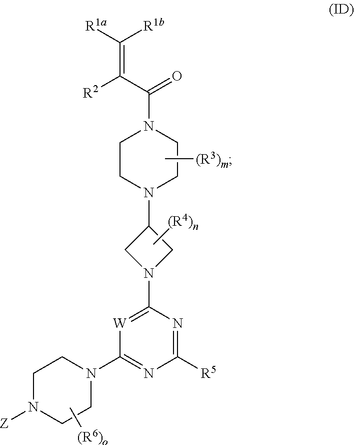

### Formula (IE):

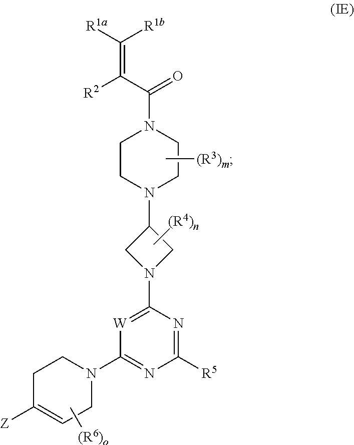

### Formula (IF):

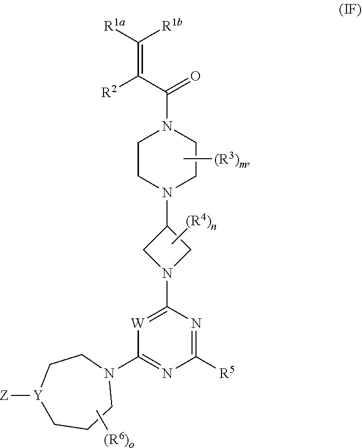

or a pharmaceutically acceptable salt of any of the foregoing.

Provided herein as Embodiment 243 is the compound of Embodiment 1, wherein the compound is a compound listed in Table A, or a pharmaceutically acceptable salt thereof.

Provided herein as Embodiment 244 is the compound of Embodiment 1, wherein the compound is a compound listed in Table E, or a pharmaceutically acceptable salt thereof.

Provided herein as Embodiment 245 is a pharmaceutical composition comprising the compound or salt of an one of Embodiments 1-244 and a pharmaceutically acceptable excipient.

Provided herein as Embodiment 246 is the compound or salt of any one of Embodiments 1-244, or the pharmaceutical composition of Embodiment 245 for use as a medicament.

Provided herein as Embodiment 247 is the compound or salt of any one of Embodiments 1-244 or the pharmaceutical composition of Embodiment 245 for use in treating cancer.

Provided herein as Embodiment 248 is the compound or salt of any one of Embodiments 1-244 or the pharmaceutical composition of Embodiment 245 for use in treating cancer, wherein one or more cancer cells express KRAS G12C mutant protein.

Provided herein as Embodiment 249 is the compound, salt, or pharmaceutical composition for use of Embodiment 247 or 248, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 250 is use of a compound or salt of any one of Embodiments 1-244 or the pharmaceutical composition of Embodiment 245 in the preparation of a medicament for treating cancer.

Provided herein as Embodiment 251 is use of a compound or salt of any one of Embodiments 1-244 or the pharmaceutical composition of Embodiment 245 in the preparation of a medicament for treating cancer, wherein one or more cancer cells express KRASG12C mutant protein.

Provided herein as Embodiment 252 is the use of Embodiment 250 or 251, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 253 is a method of treating cancer in a subject in need thereof, the method comprising administering to the subject a therapeutically effective amount of the compound or salt of any one of Embodiments 1-244, or the pharmaceutical composition of Embodiment 245.

Provided herein as Embodiment 254 is the method of Embodiment 253, wherein one or more cancer cells express KRASG12C mutant protein.

Provided herein as Embodiment 255 is the method of Embodiment 253 or 254, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 256 is the method of Embodiment 255, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, or a solid tumor.

Provided herein as Embodiment 257 is the method of Embodiment 256, wherein the cancer is non-small cell lung cancer.

Provided herein as Embodiment 258 is the method of Embodiment 256, wherein the cancer is colorectal cancer.

Provided herein as Embodiment 259 is the method of Embodiment 256, wherein the cancer is pancreatic cancer.

Provided herein as Embodiment 260 is the method of Embodiment 256, wherein the cancer is solid tumor.

Provided herein as Embodiment 261 is the method according to any one of Embodiments 255-260, wherein the subject has a cancer that was determined to have one or more cells expressing the KRAS G12C mutant protein prior to administration of the compound, salt, or pharmaceutical composition.

Provided herein as Embodiment 262 is the method according to any one of Embodiments 253-261, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents.

### EXAMPLES

The disclosure will be more fully understood by reference to the examples described herein which detail exemplary embodiments. These examples should not, however, be construed as limiting the scope of the disclosure.

This section provides specific examples of compounds of Formula I and methods of making the same.

TABLE 1
LIST OF ABBREVIATIONS

| | |
|---|---|
| Ac | acetyl |
| ACN or MeCN | acetonitrile |
| AcOH | acetic acid |
| AIBN | azobisisobutyronitrile |
| aq or aq. | aqueous |
| Bn | benzyl |
| B₂pin₂ | bis(pinacolato)diboron |
| BOC or Boc | tert-butyloxycarbonyl |
| BrettPhos | 2-(dicyclohexylphosphino)-3,6-dimethoxy-2′-4′-6′-tri-i- |
| | propyl-1,1′-biphenyl |
| Cbz | benzyloxycarbonyl |
| CbzCl | benzyl chloroformate |
| CDI | carbonyldiimidazole |
| DAST | diethylaminosulfur trifluoride |
| DBU | 1,8-diazabicyclo[5.4.0]undec-7-ene |
| DCE | dichloroethane |
| DCM | dichloromethane |
| DEA | diethyl amine |
| DIBAL | diisobutylaluminium hydride |
| DIC | N,N-diisopropylcarbodiimide |
| DMA | N,N-dimethylacetamide |
| DMAP | 4-dimethylaminopyridine |
| DMF | N,N-dimethylformamide |
| DMPU | N,N-dimethylpropyleneurea |
| DMSO | dimethyl sulfoxide |
| DPPA | diphenylphosphoryl azide |
| eq or eq. or equiv. | equivalent |
| ESI or ES | electrospray ionization |
| Et | ethyl |
| EtOAc | ethyl acetate |
| EtOH | ethanol |
| g or gr | gram(s) |
| h | hour(s) |
| HATU | 1-[bis(dimethylamino)methylene]-1H-1,2,3-triazolo[4,5- |
| | b]pyridinium 3-oxide hexafluorophosphate |
| HBpin | 4,4,5,5-tetramethyl-1,3,2-dioxaborolane |
| AcOH | acetic acid |
| HPLC | high pressure liquid chromatography |
| iPr | iso-propyl |
| iPrOH or IPA | iso-propanol |
| iPr₂NEt or DIPEA | N-ethyl diisopropylamine (Hünig′s base) |
| KOAc | potassium acetate |
| LC MS, LCMS, LC-MS or LC/MS | liquid chromatography mass spectroscopy |
| LDA | lithium diisopropylamide |
| LAH | lithium aluminium hydride |
| LHMDS or LiHMDS | lithium hexamethyldisilazide |
| m/z | mass divided by charge |
| mCPBA | meta-chloroperoxybenzoic acid |
| Me | methyl |
| MeOH | methanol |
| mg | milligrams |
| min | minutes |
| mL | milliliters |
| MS | mass spectra |
| NBS | N-bromosuccinimide |
| NCS | N-chlorosuccinimide |
| NHPI | N-hydroxyphthalimide |
| NIS | N-iodosuccinimide |
| NMP | N-methyl-2-pyrrolidinone |
| NMR | nuclear magnetic resonance |
| Pd(dppf)Cl₂, Pd(dppf)Cl₂ DCM | [1,1′- |
| | bis(diphenylphosphino)ferrocene]dichloropalladium(II), |
| | complex with dichloromethane |
| Pd(dtbpf)Cl₂ | [1,1′-bis(di-tert- |
| | butylphosphino)ferrocene]dichloropalladium(II) |
| Pd(PPh₃)₄ | tetrakis(triphenylphosphine)palladium(0) |
| Pet ether | petroleum ether |
| Ph | phenyl |
| PhMe | toluene |
| Pin | pinacolato |
| PMB | 4-methoxybenzyl |
| PTSA or pTsOH | p-toluenesulfonic acid |
| RP-HPLC | reverse phase high pressure liquid chromatography |
| RT or rt or r.t. | room temperature |
| sat. or Satd. | saturated |
| SFC | supercritical fluid chromatography |
| SPhos | 2-dicyclohexylphosphino-2′,6′-dimethoxybiphenyl |
| SPhos Pd G3 | (2-dicyclohexylphosphino-2′,6′-dimethoxybipbenyl) [2-(2′- |
| | amino-1,1′-biphenyl)]palladium(II) methanesulfonate |
| TATU | 2-(3H-[1,2,3]triazolo[4,5-b]pyridin-3-yl)-1,1,3,3- |
| | tetramethyluronium tetrafluoroborate |
| TBAF | tetra-n-butylammonium fluoride |
| TBDPS | tert-butyldiphenylsilyl |
| TBDPSCl | tert-butyldiphenylsilyl chloride |
| TBS | tert-butyldimethylsilyl |
| TBSCl | tert-butyldimethylsilyl chloride |
| tBu | tert-butyl |
| TCDI | thiocarbonyl diimidazole |
| TEA or Et₃N | triethylamine |
| temp | temperature |
| Tf | trifluoromethylsulfonyl |
| TFA | trifluoroacetic acid |
| THF | tetrahydrofuran |
| TIPS | triisopropylsilyl |
| TIPSCl | triisopropylsilyl chloride |
| TMAF | tetramethylammonium fluoride |
| UV | ultraviolet |
| XantPhos | (9,9-dimethyl-9H-xanthene-4,5- |
| | diyl)bis(diphenylphosphane) |
| XantPhos Pd G3 | [(4,5-bis(diphenylphosphino)-9,9-dimethylxanthene)-2-(2′- |
| | amino-1,1′-biphenyl)]palladium(II) |
| XPhos | 2-dicyclohexylphosphino-2′,4′,6′-triisopropyl-1,1′-biphenyl |
| XPhos Pd G2 | chloro(2-dicyclohexylphosphino-2′,4′,6′-triisopropyl-1,1′- |
| | biphenyl)[2-(2′-amino-1,1′-biphenyl)]palladium(II) |

Provided in this section are descriptions of the general analytical and purification methods used to prepare the specific examples provided herein.

Chromatography: Unless otherwise indicated, crude product-containing residues were purified by passing the crude material or concentrate through either a Biotage or ISCO brand silica gel column pre-packed with flash silica (SiO₂) and eluting the product off the column with a solvent gradient as indicated.

Preparative HPLC Method: Where indicated, the compounds described herein were purified via reverse phase HPLC using Waters FractionLynx or Gilson semi-preparative HPLC-MS system utilizing one of the following two HPLC columns: (a) Phenomenex Gemini column (5 micron, C18, 150×30 mm) or (b) Waters X-select CSH column (5 micron, C18, 100×30 mm). A typical run through the instrument included: eluting at 45 mL/min with a linear gradient of 10% (v/v) to 100% MeCN (0.1% v/v formic acid) in H₂O (0.1% formic acid) over 10 min; conditions can be varied to achieve optimal separations.

Proton NMR Spectra: Unless otherwise indicated, all ¹H NMR spectra were collected on a Bruker NMR instrument at 300, 400 or 500 MHz. All observed protons are reported as parts-per-million (ppm) downfield from tetramethylsilane (TMS) using the internal solvent peak as reference. Some ¹H signals may be missing due to exchange with D from CD₃OD, or due to signal suppression.

Mass Spectra (MS): Unless otherwise indicated, all mass spectral data for starting materials, intermediates and/or exemplary compounds are reported as mass/charge (m/z), having an [M+H]+ molecular ion. The molecular ion reported was obtained by electrospray detection method (commonly referred to as an ESI MS) utilizing a Waters Acquity UPLC/MS system. Compounds having an isotopic atom, such as bromine and the like, are generally reported according to the detected isotopic pattern, as appreciated by those skilled in the art.

### Section 1: Preparation of Intermediates

### 5-Iodo-1-(2-methoxyethyl)-4-methyl-1H-pyrazole and 3-iodo-1-(2-methoxyethyl)-4-methyl-1H-pyrazole (Intermediate A-1 and A-2)

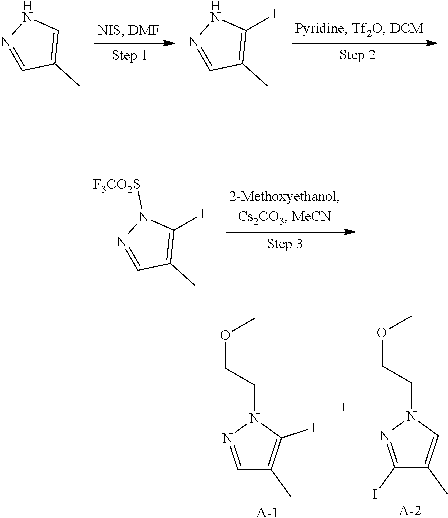

Step 1. To a solution of 4-methyl-1H-pyrazole (500 g, 6.1 mol, Arbor) in DMF (5000 mL) at rt was added NIS (1370 g, 6.1 nmol, Spectrochem) and the mixture was heated at 65° C. for 1 h. The reaction mixture was quenched with crushed ice (10 L) and extracted with tert-butyl methyl ether (3×5 L). The organic extract was washed with Satd. aq. sodium thiosulphate (5 L), brine (5 L), dried over Na₂SO₄, filtered, and concentrated wider reduced pressure. The crude residue was purified by column chromatography (silica, 10 to 20% EtOAc:hexanes) to give 5-iodo-4-methyl-1H-pyrazole. m/z (ESI): 209.1 (M+H)⁺. ¹H NMR (400 MHz, DMSO-d₆) δ ppm 12.% (s, 1H), 7.50 (s, 1H), 1.92 (s, 3H).

Step 2. To a mixture of 5-iodo-4-methyl-1H-pyrazole (158 g, 760 mmol) and pyridine (92 mL, 1139 mmol. Sonia Industries) in DCM (1580 mL) at 0° C. was slowly added trifluoromethanesulfonic anhydride (154 mL, 912 mmol, Avra Synthesis) and the mixture was stirred at rt for 30 min. The reaction was quenched by addition into ice-cold water (3000 mL) and extracted with DCM (2 L). The organic extract was washed with brine (2 L), dried over Na₂SO₄, filtered, and concentrated under reduced pressure to give 5-iodo-4-methyl-1-((trifluoromethyl)sulfonyl)-1H-pyrazole. m/z (ESI): No ionization. ¹H NMR (400 MHz, DMSO-d₆) δ ppm 8.44 (s, 1H), 2.02 (s, 3H).

Step 3. To a mixture of 5-iodo-4-methyl-1-((trifluoromethyl)sulfonyl)-1H-pyrazole (360 g, 1059 mmol) and Cs₂CO₃ (517 g, 1588 mmol, Avra Synthesis) in MeCN (3600 mL) at 0° C. was added 2-methoxyethan-1-ol (100 mL, 1270 mmol, TCI) dropwise. The reaction mixture was stirred at rt for 1 h. The reaction mixture was quenched with H₂O (5 L), extracted with EtOAc (3 L). The organic extract as washed with brine (3000 mL), dried over Na₂SO₄, filtered, and concentrated under reduced pressure. The crude residue was purified by column chromatography (silica, 10 to 20% EtOAc:hexanes) to give a mixture of 5-iodo-1-(2-methoxyethyl)-4-methyl-1H-pyrazol and 3-iodo-1-(2-methoxyethyl)-4-methyl-1H-pyrazole. The regioisomers were separated by SFC (Chiral Pak IC (150×50 mm, 5μ) with a mobile phase of 90% CO₂ and 10% MeOH using a flow rate of 150 mL/min) to give 5-iodo-1-(2-methoxyethyl)-4-methyl-1H-pyrazole) (1^{st} peak, Int A-1). m/z (ESI): 267.1 (M+H)⁺. ¹H NMR (400 MHz DMSO-d₆) δ ppm 7.42 (s, 1H), 4.25 (t, 2H, J=5.7 Hz), 3.66 (t, 2H, J=5.7 Hz), 3.21 (s, 3H), 1.95 (s, 3H) and 3-iodo-1-(2-methoxyethyl)-4-methyl-1H-pyrazole (2^{nd} peak, Int A-2). m/z (ESI): 267.1 (M+H)⁺. ¹H NMR (400 MHz, DMSO-d₆) δ ppm 7.47 (s, 1H), 4.20 (t, 2H, J=5.3 Hz), 3.63 (t, 2H, J=5.3 Hz), 3.22 (s, 3H), 1.89 (s, 3H).

The intermediates in the table below were prepared in a fashion similar to that described above for intermediate A-1.

| | | | m/z (ESI): |
|---|---|---|---|
| Int. # | Chemical Structure | Name | (M + H)⁺ |
| A-3 | 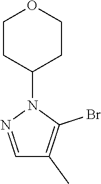 | 5-bromo-4-methyl-1- (tetrahydro-2H-pyran-4- yl)-1H-pyrazole | 244.9 |
| A-4 | 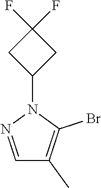 | 5-bromo-1-(3,3- difluorocyclobutyl)- 4-methyl- 1H-pyrazole | 250.9 |
| A-5 | 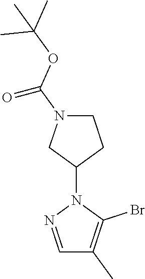 [SMILES: Cc1cnn(C2CCN(C(=O)OC(C)(C)C)C2)c1Br] | tert-butyl 3-(5-bromo- 4-methyl-1H-pyrazol-1- yl)pyrrolidine-1- carboxylate | NA |
| A-6 | 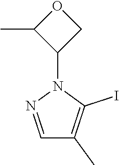 | 5-iodo-4-methyl-1-(2- methyloxetan-3-yl)-1H- pyrazole | 279.6 |
| A-7 | 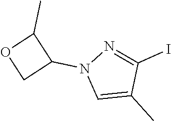 | 3-iodo-4-methyl-1-(2- methyloxetan-3-yl)-1H- pyrazole | 279.6 |

### 5-Bromo-4-methyl-1-(oxetan-3-yl)-1H-pyrazole (intermediate A-8)

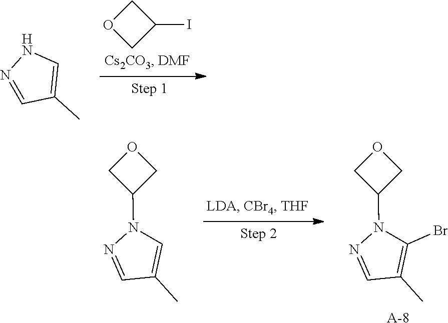

Step 1. To a mixture of 4-methyl-1H-pyrazole (50.0 g, 609 mmol, Combi-Blocks, Inc.) and cesium carbonate (397 g, 1218 mmol, Chempure) in DMF (750 mL) was added 3-iodooxetane (168 g, 913 mmol, Oakwood) and the mixture was heated at 80° C. for 16 h. The mixture was cooled to rt, quenched with H₂O (3000 mL), and extracted with EtOAc (1000 mL). The organic extract was washed with brine, concentrated, and then purified by chromatography (silica, 0 to 20% EtOAc:hexanes) to give methyl 4-methyl-1-(oxetan-3-yl)-1H-pyrazole. m/z (ESI): 139.2 (M+H)⁺. ¹H NMR (400 MHz, DMSO-d₆) δ ppm 7.64 (s, 1H), 7.37 (s, 1H), 5.4-5.5 (m, 1H), 4.7-4.9 (m, 4H), 2.02 (s, 3H).

Step 2. LDA (2 M solution in THF, 326 mL, 651 mmol, Sigma-Aldrich. Inc.) was added to a solution of 4-methyl-1-(oxetan-3-yl)-1H-pyrazole (60 g, 434 mmol) in THF (750 mL) at −78° C., and the mixture was stirred for 30 min. Carbon tetrabromide (216 g, 651 mmol, TCI) in THF (500 mL) was added dropwise over 1 h at −78° C., and the mixture was stirred for another 1 h. The reaction mixture was quenched with satd. aq. NH₄Cl (1500 mL) and extracted with EtOAc (600 mL). The organic extract was dried with Na₂SO₄, concentrated, and then purified by chromatography (silica, 0 to 15% EtOAc:hexanes) to give 5-bromo-4-methyl-1-(oxetan-3-yl)-1H-pyrazole. m/z (ESI): 218.9 (M+H)⁺. ¹H NMR (400 MHz, DMSO-d₆) δ ppm 7.59 (s, 1H), 5.61 (p, 1H, J=6.9 Hz), 4.90 (d, 4H, J=6.9 Hz), 1.97 (s, 3H).

The intermediates in the table below were prepared in a fashion similar to that described above for intermediate A-8.

| | | | m/z (ESI): |
|---|---|---|---|
| Int. # | Chemical Structure | Name | (M + H)⁺ |
| A-9 | 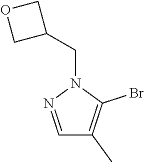 | 5-bromo-4-methyl- 1-(oxetan-3-ylmethyl)-1H- pyrazole | 231.1 |
| A-10 | 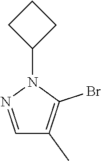 | 5-bromo-1- cyclobutyl-4-methyl- 1H-pyrazole | 214.9 |

### 5-Chloro-4-nitro-1-(oxetan-3-yl)-1H-pyrazole (Intermediate A-11)

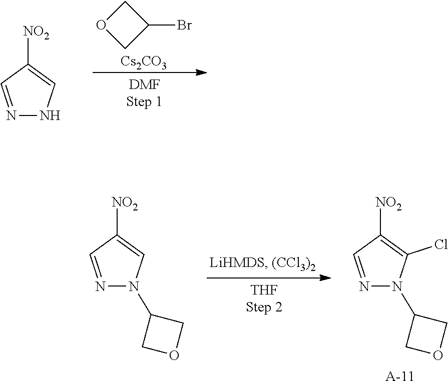

Step 1. To a solution of 4-nitro-1H-pyrazole (2.7 g, 23.88 mmol, Combi-Blocks, Inc.) in DMF (30 mL) was added 3-bromooxetane (4.25 g, 31.0 mmol, Ambeed, Inc.) and cesium carbonate (1945 g, 59.7 mmol). The mixture was stirred for 16 h at 100° C. The reaction was brought to rt, diluted with H₂O (150 mL) and extracted with EtOAc. The organic extract was washed with brine, dried over Na₂SO₄, filtered, concentrated, and chromatographed on silica gel eluting with 0% to 30% (3:1 EtOAc/EtOH) in heptane to afford 4-nitro-1-(oxetan-3-yl)-1H-pyrazole. m/z (ESI): 170.0 (M+H)⁺.

Step 2. To a −78° C. solution of LiHMDS (1 M in THF, 45.4 mL, 45.4 mmol, Sigma-Aldrich, Inc.) in THF (100 mL) was added a solution of 4-nitro-1-(oxetan-3-yl)-1H-pyrazole (3.84 g, 22.7 mmol) in THF (20 mL) dropwise and the reaction mixture was stirred at −78° C. for 20 min. To the reaction mixture was added a solution of hexachloroethane (6.44 g, 27.24 mmol, Oakwood Products, Inc.) in THF (20 mL), and the reaction mixture was stirred at −78° C. for 20 min before being warmed to rt and stirred for an additional 30 min. The reaction was quenched by the addition of sat. aq. NH₄Cl and extracted with EtOAc. The organic extracts were dried over Na₂SO₄ and concentrated. To the residue obtained was added DCM. The suspension was filtered and the filtrate was concentrated to afford 5-chloro-4-nitro-1-(oxetan-3-yl)-1H-pyrazole. m/z (ESI): 204.2 (M+H)⁺.

The intermediates in the table below were prepared in a fashion similar to that described above for intermediate A-11.

| | | | m/z (ESI): |
|---|---|---|---|
| Int. # | Chemical Structure | Name | (M + H)⁺ |
| A-12 | 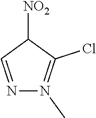 | 5-chloro-1-methyl- 4-nitro-1H-pyrazole | 162.0 |
| A-13 | 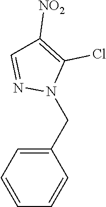 [SMILES: O=[N+]([O-])c1cnn(Cc2ccccc2)c1Cl] | 1-benzyl-5-chloro- 4-nitro-1H-pyrazole | 238.0 |
| A-14 | 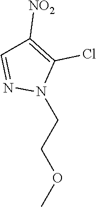 | 5-chloro-1-(2- methoxyethyl)-4-nitro-1H- pyrazole | 206.2 |

### 3-Chloro-2-(oxetan-3-yl)pyridine (Intermediate A-15)

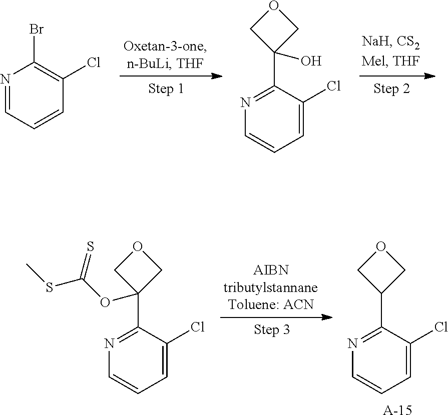

Step 1. To a stirred solution of 2-bromo-3-chloropyridine (15 g, 78 mmol, Combi-Blocks, Inc.) in THF (150 mL) at −78° C. was added n-BuLi (1.6 M in hexanes. 49.7 mL, 80 mmol, Symax Ltd) dropwise and the mixture was stirred for 30 min. A solution of oxetan-3-one (5.06 g, 70.2 mmol, Combi-Blocks, Inc.) in THF (20 mL) was added, and the reaction mixture was stirred again for 1.5 h. The reaction mixture was quenched with Satd. aq. NH₄Cl (100 mL) and extracted with EtOAc (100 mL). The organic extract was washed with brine (100 mL), dried over Na₂SO₄, filtered, and concentrated. The residue was purified on a Redi-Sep pre-packed silica gel column eluting with 25-35% EtOAc in hexanes to give 3-(3-chloropyridin-2-yl)oxetan-3-ol. m/z (ESI): 186.1 (M+H)⁺. ¹H NMR (400 MHz, DMSO-d₆): δ ppm 8.52 (dd, 1H, J=4.7, 1.4 Hz), 7.96 (dd, 1H, J=8.1, 1.4 Hz), 7.44 (dd, 1H, J=8.0, 4.7 Hz), 6.51 (s, 1H), 5.16 (d, 2H, J=6.9 Hz), 4.70 (d, 2H, J=7.0 Hz).

Step 2. To a stirred solution of 3-(3-chloropyridin-2-yl)oxetan-3-ol (3 g, 16.16 mmol) in THF (30 mL) at 0° C. was added NaH (0.996 g, 24.89 mmol, 60% dispersion in mineral oil, Spectrochem). The reaction mixture was stirred for 30 min at 0° C. Carbon disulfide (1.46 mL, 24.24 mmol, Chempure) in THF (2 mL) was added dropwise, and the reaction mixture was stirred at 0° C. for 1.5 h. Methyl iodide (1.52 mL, 24.24 mmol, Spectrochem) was added and the reaction mixture was stirred at 0° C. for 1 h. The reaction mixture was quenched with ice cold water (30 mL) and extracted with EtOAc (50 mL). The organic extract was washed with brine (50 mL), dried over Na₂SO₄, filtered, and concentrated under reduced pressure to provide 0-(3-(3-chloropyridin-2-yl)oxetan-3-yl) S-methyl carbonodithioate. m/z (ESI): 276.0 (M+H)⁺.

Step 3. To a stirred solution of O-(3-(3-chloropyridin-2-yl)oxetan-3-yl) S-methyl carbonodithioate (3 g, 10.88 mmol) in toluene (60 mL) was added a solution of AIBN (0.182 g, 1.110 mmol, Chempure) in MeCN (1.2 mL), and the mixture was degassed for 5 min. Tributylstannane (6.33 g, 21.76 mmol, Sigma-Aldrich Corporation) was added to the reaction mixture, and the mixture was stirred at 100° C. for 2 h. The reaction mixture was quenched with Satd. aq. NH₄Cl (100 mL) and extracted with EtOAc (100 mL). The organic extract was washed with brine, dried over Na₂SO₄, filtered, and concentrated under reduced pressure. The residue was purified on a Redi-Sep pre-packed silica gel column (25 g) eluting with 35% to 45% EtOAc in pet ether to provide 3-chloro-2-(oxetan-3-yl)pyridine. m/z (ESI): 170.3 (M+H)⁺. ¹H NMR (400 MHz, Chloroform-d): δ ppm 8.57 (dd, 1H, J=4.7, 1.5 Hz), 7.68 (dd, 1H, J=8.0, 1.5 Hz), 7.20 (dd, 1H, J=8.0, 4.7 Hz), 5.1-5.2 (m, 4H), 4.7-4.9 (m, 1H).

### 4-Bromo-3,5-dimethylisothiazole (Intermediate A-16)

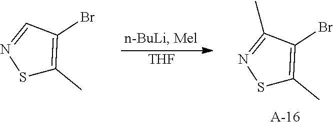

To a −78° C. solution of 4-bromo-3-methyl-1,2-thiazole (1 g, 0.59 mL, 5.62 mmol, Enamine) in THF (4 mL) was added n-butyllithium (2.5 M in THF, 2.70 mL, 6.74 mmol, Sigma-Aldrich, Inc.), and the reaction mixture was stirred at −78° C. for 15 min. To the reaction mixture was added iodomethane (0.420 mL, 6.74 mmol, Sigma-Aldrich, Inc.), and the reaction mixture was stirred at −78° C. for 5 min before being warmed to rt and stirred for an additional 15 min. The reaction mixture was diluted with Satd. aq. NH₄Cl and extracted with EtOAc. The organic extract was filtered through a plug of anhydrous Na₂SO₄ and concentrated in vacuo. The crude material was purified by chromatography through a Redi-Sep pre-packed silica gel column (24 g), eluting with a gradient of 0% to 20% EtOAc in heptane, to provide 4-bromo-3,5-dimethylisothiazole. m/z (ESI): 194.1 (M+H)⁺.

### 4-Bromo-5-methyl-3-(oxetan-3-yl)isothiazole (Intermediate A-17)

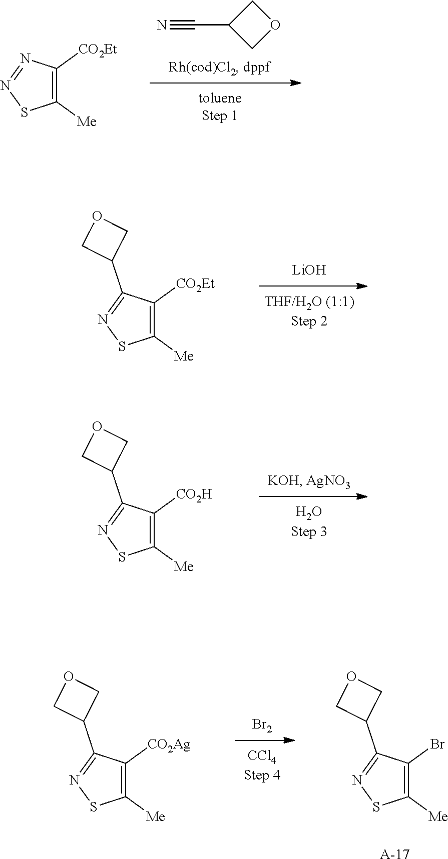

Step 1. To a vial was added chloro(1,5-cyclooctadiene)rhodium(i) dimer (79 mg, 0.160 mmol, Combi-Blocks. Inc.), dppf (212 mg, 0.383 mmol, Strem Chemicals, Inc.), and 5-methyl-[1,2,3]thiadiazole-4 carboxylic acid ethyl ester (550 mg, 3.19 mmol, Aurum Pharmatech LLC). The reaction vial was evacuated and backfilled with N₂. To the vial was added toluene (5 mL) and 3-oxetanecarbonitrile (1.10 mL, 13.25 mmol, Enamine) and the reaction mixture was heated to 100° C. and stirred for 1 h. The reaction mixture was cooled to rt and concentrated in vacuo. The crude material was purified by chromatography through a Redi-Sep pre-packed silica gel column, eluting with a gradient of 0% to 20% EtOAc in heptane, to provide ethyl 5-methyl-3-(oxetan-3-yl)isothiazole-4 carboxylate. m/z (ESI): 228.2 (M+H)⁺.

Step 2. To a vial was added ethyl 5-methyl-3-(oxetan-3-yl)isothiazole-4-carboxylate (611 mg, 2.69 mmol), lithium hydroxide monohydrate (564 mg, 13.44 mmol, Combi-Blocks. Inc.), THF (1 mL), and H₂O (1 mL), and the reaction mixture was heated to 80° C., and stirred for 2 h. The reaction mixture was cooled to rt, and 1.0 M aqueous HCl was added until the mixture measured a pH<3. The reaction mixture was extracted with EtOAc. The organic extract was washed with brine, dried over anhydrous Na₂SO₄, filtered, and concentrated in vacuo to give crude 5-methyl-3-(oxetan-3-yl)isothiazole-4-carboxylic acid that was used without further purification. m/z (ESI): 200.2 (M+H)⁺.

Step 3. To a suspension of 5-methyl-3-(oxetan-3-yl)isothiazole-4-carboxylic acid (0.17 g, 0.832 mmol) in H₂O (2 mL) was added a solution of potassium hydroxide (0.560 g, 0.998 mmol, Sigma-Aldrich, Inc.) in H₂O (2 mL), and the reaction mixture was stirred until a homogeneous solution was observed. To the reaction mixture was added a solution of silver nitrate (0.170 g, 0.998 mmol, Sigma-Aldrich, Inc.) in H₂O (2 mL, and the reaction mixture was stirred at rt until a gray/white precipitate was observed. The precipitate was filtered and rinsed with H₂O followed by acetone. The precipitate was dried under vacuum at 50° C. for 30 min to give ((5-methyl-3-(oxetan-3-yl)isothiazole-4-carbonyl)oxy)silver.

Step 4. To a suspension of ((5-methyl-3-(oxetan-3-yl)isothiazole-4-carbonyl)oxy)silver (0.11 g, 0.366 mmol) in carbon tetrachloride (2 mL) was added bromine (0.022 mL, 0.439 mmol, Sigma-Aldrich. Inc.), and the reaction mixture was stirred at rt for 1 h. The reaction mixture was filtered, and the filtrate was washed with Satd. aq. Na₂S₂O₈ and extracted with DCM. The organic extract was washed with brine, dried over Na₂SO₄, filtered, and concentrated in vacuo. The crude material was purified by chromatography through a Redi-Sep pre-packed silica gel column (12 g), eluting with a gradient of 0% to 20% EtOAc in heptane, to provide 4-bromo-5-methyl-3-(oxetan-3-yl)isothiazole. m/z (ESI): 234.0 (M+H)⁺.

### 4-Bromo-3-methyl-5-(oxetan-3-yl)isothiazole (Intermediate A-18)

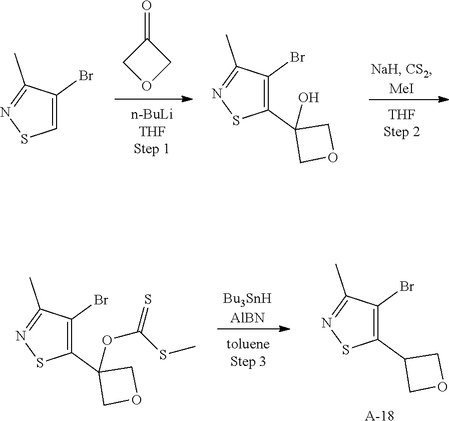

Step 1. To a −78° C. solution of 4-bromo-3-methylisothiazole (1 g, 5.62 mmol, Enamine) in THF (6 mL) was added n-BuLi (2.5 M in hexanes, 2.25 mL, 5.62 mmol, Sigma-Aldrich, Inc.) and the reaction mixture was stirred at −78° C. for 15 min. To the reaction mixture was added oxetan-3-one (0.360 mL, 5.62 mmol, Combi-Blocks, Inc.) and the reaction mixture was stirred for 5 min at −78° C. before being warmed to rt and stirred for 15 min. The reaction mixture was quenched with Satd. aq. NH₄Cl and extracted with EtOAc. The organic extract was filtered through a plug of Na₂SO₄ and concentrated in vacuo. The crude material was triturated with heptane and filtered and collected to provide 3-(4-bromo-3-methylisothiazol-5-yl)oxetan-3-ol. m/z (ESI): 250.0 (M+H)⁺.

Step 2: To a suspension of NaH (0.173 g, 4.33 mmol, 60% dispersion in mineral oil, Oakwood Products, Inc.) in THF (1 mL) at 0° C. was added a solution of 3-(4-bromo-3-methylisothiazol-5-yl)oxetan-3-ol (0.72 g, 2.89 mmol) in THF (1 mL), and the reaction mixture was stirred at 0° C. for 15 min. To the reaction mixture was added carbon bisulfide (0.261 mL, 4.33 mmol, Sigma-Aldrich, Inc.), and the reaction mixture was stirred at 0° C. for an additional 15 min. To the reaction mixture was added iodomethane (0.27 mL, 4.33 nmol. Sigma-Aldrich, Inc.), and the reaction mixture was stirred at 0° C. for 15 min and then warned to n. The reaction mixture was quenched by the slow addition of H₂O and extracted with EtOAc. The organic extract was washed with brine, dried over Na₂SO₄, filtered. and concentrated in vacuo to provide crude 0-(3-(4-bromo-3-methylisothiazol-5-yl)oxetan-3-yl) S-methyl carbonodithioate that was used without further purification assuming quantitative yield. m/z (ESI): 340.0 (M+H)⁺.

Step 3. To a vial was added O-(3-(4-bromo-3-methylisothiazol-5-yl)oxetan-3-yl) S-methyl carbonodithioate (0.46 g, 1.337 mmol) and 2,2′-azobis(2-methylpropionitrile) (0.022 g, 0.134 mmol, Sigma-Aldrich, Inc.). The reaction vessel was evacuated and backfilled with N₂ before toluene (4.46 mL) was added. The reaction mixture was sparged with N₂ for 10 min before tributyltin hydride (0.584 g, 0.540 mL, 2.0 mmol, Sigma-Aldrich, Inc.) was added. The reaction was heated to 100° C., and stirred for 2 h. The reaction mixture was diluted with brine and extracted with EtOAc. The organic extract was dried over anhydrous Na₂SO₄, filtered, and concentrated in vacuo. The crude material was purified by chromatography through a Redi-Sep pre-packed silica gel column (12 g), eluting with a gradient of 0% to 20% EtOAc in heptane, to provide 4-bromo-3-methyl-5-(oxetan-3-yl)isothiazole. m/z (ESI): 234.0 (M+H)⁺.

### 3,5-Dimethyl-4-(4,4,5,5-tetramethyl-1,3,2-dioxaborolan-2-yl)isothiazole (Intermediate A-19)

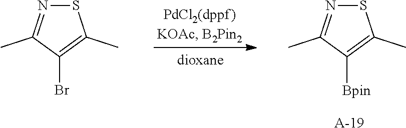

To a vial was added bis(pinacolato)diboron (2.89 g, 11.39 mmol, Combi-Blocks, Inc.). potassium acetate (1.118 g, 11.39 mmol, Sigma-Aldrich, Inc.), and Pd(dppf)Cl₂ (0.313 g, 0.427 mmol, Sigma-Aldrich, Inc.). The reaction vessel was evacuated and backfilled with N₂. To the reaction vessel was added a solution of 4-bromo-3,5-dimethylisothiazole (1.094 g, 5.70 mmol) in 1,4-dioxane (10 mL), and the reaction mixture was heated to 100° C., and stirred for 18 h. The reaction mixture was filtered and concentrated in vacuo. The crude material was purified by chromatography through a Redi-Sep pre-packed silica gel column, eluting with a gradient of 0% to 5% EtOAc in heptane, to provide 3,5-dimethyl-4-(4,4,5,5-tetramethyl-1,3,2-dioxaborolan-2-yl)isothiazole. m/z (ESI): 240.2 (M+H)⁺.

### 1-(5-Bromo-4-methyl-1H-pyrazol-1-yl)-2-methylpropan-2-ol (Intermediate A-20)

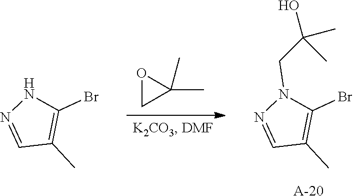

To a stirred solution of 5-bromo-4-methyl-1H-pyrazole (4 g, 24.84 mmol) in DMF (40 mL) at rt was added 2,2-dimethyloxirane (2.150 g, 29.8 mmol) and K₂CO₃ (8.58 g, 62.1 mmol). The reaction mixture was stirred at 100° C. for 16 h. The reaction mixture was cooled to rt, diluted with ice cold water (300 mL), and extracted with EtOAc (2-150 mL). The combined organic extracts were washed with sat. aq. NaCl (200 mL) and dried over Na₂SO₄. The mixture was filtered, concentrated, and purified by chromatography through a Redi-Sep pre-packed silica gel column, eluting with a gradient of 10% to 15% EtOAc in hexanes, to provide 1-(5-bromo-4-methyl-1H-pyrazol-1-yl)-2-methylpropan-2-ol. m/z (ESI): 235.0 (M+H)⁺.

### 5-Bromo-1-cyclopropyl-4-methyl-1H-pyrazole (Intermediate A-21)

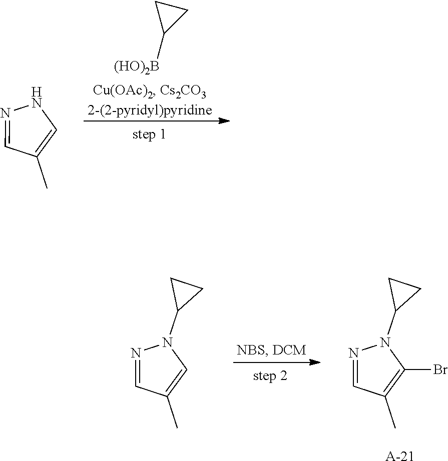

A mixture of 4-methyl-1H-pyrazole (30.0 g, 365 mmol, Bide Pharmatech Ltd., China). 2-(2-pyridyl)pyridine (5.71 g, 36.5 mmol, Bide Pharmatech Ltd., China). Cu(OAc)₂ (66.4 g, 365 mmol), Cs₂CO₃ (119 g, 365 mmol) and cyclopropylboronic acid (62.8 g, 731 mmol) in DCE (500 mL) was degassed and purged with N₂ three times. The reaction mixture was stirred at 70° C. for 2 h under a N₂ atmosphere. The reaction mixture % as concentrated under reduced pressure and purified by column chromatography (SiO₂, 0% to 10% EtOAc in pet ether) to give 1-cyclopropyl-4-methyl-1H-pyrazole.

Step 2: To a solution of 1-cyclopropyl-4-methyl-1H-pyrazole (5.0 g, 40.9 mmol) in DCM 1100 mL) was added NBS (11.65 g, 65.5 mmol). The mixture was stirred at 20° C. for 16 h. The reaction mixture—as concentrated under reduced pressure and the residue was purified by column chromatography (SiO₂, 0% to 10% EtOAc in pet ether) to provide 5-bromo-1-cyclopropyl-4-methyl-1H-pyrazole. m/z (ESI): 201.1 (M+H)⁺. ¹H NMR (400 MHz, CHLOROFORM-d) δ ppm 7.29 (s, 1H), 3.41-3.47 (m, 1H), 3.01 (s, 3H), 1.15-1.20 (m, 2H), 1.01-1.08 (m, 2H).

### 1-Cyclopropyl-5-iodo-4-methyl-1H-imidazole (Intermediate A-22)

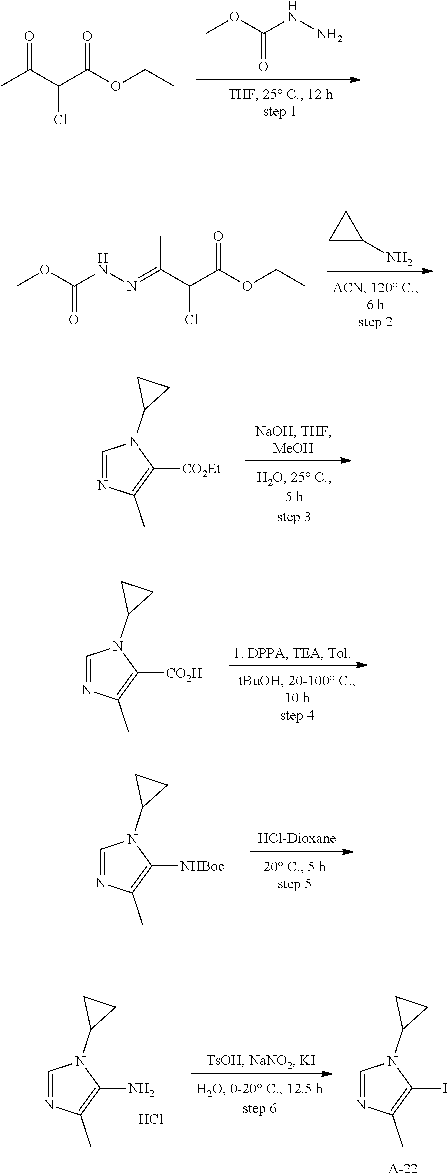

Step 1: Ethyl 2-chloro-3-oxo-butanoate (84.03 mL, 607.58 mmol, WuXi AppTec, China) and methyl N-aminocarbamate (54.73 g, 607.58 nmol, WuXi AppTec, China) were dissolved in THF (2002 mL) at 25° C. under N₂. The mixture was stirred at 25° C. for 12 h, and the reaction mixture was concentrated under reduced pressure. The residue was diluted with H₂O (500 mL) and extracted with EtOAc (3-200 mL). The combined organic extracts were dried over Na₂SO₄, filtered, and concentrated under reduced pressure to give ethyl (3E)-2-chloro-3-(methoxycarbonylhydrazono)butanoate, which was carried forward to next step.

Step 2: Cyclopropanamine (58.56 mL, 845.12 mmol, WuXi AppTec. China) and ethyl (3E)-2-chloro-3-(methoxy carbonylhydrazone)butanoate (100 g, 422.56 mmol) in ACN (2001 mL) were heated at 120° C. for 6 h. The reaction mixture was concentrated under reduced pressure, diluted with H₂O (500 mL), and extracted with EtOAc (3×200 mL). The combined organic extracts were dried over Na₂SO₄. filtered, and concentrated. The residue was purified by column chromatography (SiO₂, 0% to 50% EtOAc in pet ether) to provide ethyl 3-cyclopropyl-5-methyl-imidazole-4-carboxylate. m/z (ESI): 195.1 (M+H)⁺.

Step 3: Ethyl 3-cyclopropyl-5-methyl-imidazole-4-carboxylate (16.5 g, 84.95 mmol) was dissolved in MeOH (50 mL), THF (50 mL) and H₂O (50 mL). NaOH (6.80 g, 169.90 mmol) was added in portions at 25° C. under N₂. The mixture was stirred at 25° C. for 5 h. HCl (1N aqueous, 150 mL) was added slowly to the reaction mixture, and the mixture was then extracted with EtOAc (3×100 mL). The combined organic extracts were dried over Na₂SO₄, filtered, and concentrated under reduced pressure to give 3-cyclopropyl-5-methyl-imidazole-4-carboxylic acid that was carried on directly to the next step.

Step 4: A mixture of 3-cyclopropyl-5-methyl-imidazole-4-carboxylic acid (25.0 g, 150 mmol), TEA (45.7 g, 451 mmol) and DPPA (62.1 g, 226 mmol) in toluene (200 mL) was degassed and purged with N₂ three times. The reaction mixture was stirred at 20° C. for 1 h under a N₂ Atmosphere. tert-Butanol (200 mL) was added, and the resulting mixture was stirred at 100° C. for 9 h. The reaction mixture was diluted with H₂O (1000 mL) and extracted with EtOAc (3×500 mL). The combined organic extracts were dried over Na₂SO₄, filtered, and concentrated under reduced pressure. The residue was purified by column chromatography (SiO₂, 0% to 100% EtOAc in pet ether) to provide tert-butyl (1-cyclopropyl-4-methyl-1H-imidazol-5-yl)carbamate.

Step 5: A mixture of tert-butyl (1-cyclopropyl-4-methyl-1H-imidazol-5-yl)carbamate (13.0 g, 54.8 mmol) and HCl in dioxane (4 M, 100 mL, 400 mmol) was stirred at 20° C. for 5 h. The reaction mixture was concentrated under reduced pressure, and 1-cyclopropyl-4-methyl-1H-imidazol-5-amine hydrochloride was obtained. m/z (ESI): 238.1 (M+H)⁺.

Step 6: To a solution of 1-cyclopropyl-4-methyl-1H-imidazol-5-amine hydrochloride (5.5 g, 31.7 mmol) in H₂O (80 mL) was added a solution of toluenesulfonic acid (10.91 g, 63.3 mmol) in H₂O (80 mL) followed by a solution of NaNO₂ (3.28 g, 47.5 mmol) in H₂O (80 mL). The mixture was stirred at 0° C. for 0.5 h before KI (26.3 g, 158 mmol) in H₂O (80 mL) was added dropwise at 0° C. The resulting mixture was stirred at 20° C. for 12 h. The reaction mixture was extracted with EtOAc (3×500 mL) The combined organic extracts were dried over Na₂SO₄, filtered, and concentrated under reduced pressure. The residue was purified by column chromatography (SiO₂, 0% to 100% EtOAc in pet ether) to provide 1-cyclopropyl-5-iodo-4-methyl-1H-imidazole. m/z (ESI): 249.1 (M+H)⁺.

### 4-Chloro-3-methyl-5,7-dihydrofuro[3,4-b]pyridine (Intermediate A-23)

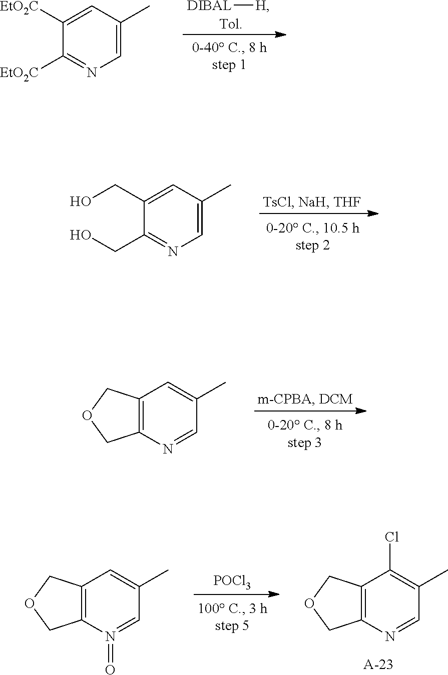

Step 1: To a solution of diethyl 5-methylpyridine-2,3-dicarboxylate (12.5 g, 52.7 mmol, Jiangsu Aikon Biopharmaceutical R & D, China) in toluene (125 mL) at 0° C. was added dropwise DIBAL-H (263 mL, 263 mmol). After addition. the mixture was stirred at 40° C. for 8 h. The reaction mixture was cooled to 0° C., and quenched by addition of H₂O (150 mL) and aqueous NaOH (15%, 150 mL). The mixture was filtered, and the filter cake was washed with MeOH (5000 mL). The filtrate was concentrated under reduced pressure. The resulting residue was purified by column chromatography (SiO₂, 50% to 100% EtOAc in pet ether, then 0% to 10% MeOH in EtOAc) to give (5-methylpyridine-2,3-diyl)dimethanol.

Step 2: To a solution of (5-methylpyrimidin-2,3-diyl)dimethanol (3.0 g, 19.58 mmol) in THF (10 mL) at 0° C. was added NaH (0.783 g, 19.58 mmol, 60% dispersion in mineral oil). After addition, the mixture was stirred at 0° C. for 0.5 h before 4-methylbenzenesulfonyl chloride (3.73 g, 19.58 mmol) in THF (10 mL) was added dropwise. The resulting mixture was stirred at 20° C. for 5 h. Additional NaH (1.175 g, 29.4 mmol, 60% dispersion in mineral oil) was added, and the mixture was stirred at 20° C. for 5 h. The reaction mixture was cooled to 0° C., quenched by addition of H₂O (50 mL), and extracted with EtOAc (2×50 mL). The combined organic extracts were dried over Na₂SO₄, filtered, and concentrated under reduced pressure to give a residue. The residue was purified by column chromatography (SiO₂, 0% to 100% EtOAc in pet ether) to give 3-methyl-5,7-dihydrofuro[3,4-b]pyridine.

Step 3: To a solution of 3-methyl-5,7-dihydrofuro[3,4-b]pyridine (1.5 g, 11.10 mmol) in DCM (30 mL) was added m-CPBA (3.11 g, 14.43 mmol) at 0° C., and the mixture was stirred at 20° C. for 8 h. The reaction mixture cooled to 0° C., quenched by addition of sat. aq. Na₂SO₃ (50 mL), and extracted with EtOAc (2×30 mL). The combined organic extracts were dried over Na₂SO₄, filtered, and concentrated under reduced pressure. The residue was purified by column chromatography (SiO₂, 0% to 10% MeOH in EtOAc) to give 3-methyl-5,7-dihydrofuro[3,4-b]pyridine 1-oxide.

Step 4: A solution of 3-methyl-5,7-dihydrofuro[3,4-b]pyridine 1-oxide (1.5 g, 9.92 mmol) in POCl₃ (17 mL, 182 mmol) was stirred at 100° C. for 3 h. The reaction mixture was concentrated under reduced pressure. The mixture was quenched by the addition of H₂O (20 mL) at 20° C., adjusted to pH=7 with sat. aq. Na₂CO₃, and extracted with EtOAc (2-20 mL). The combined organic extracts were washed with brine (2×20 mL), dried over Na₂SO₄, filtered, and concentrated under reduced pressure to give a residue. The residue was purified by column chromatography (SiO₂, 0% to 30% EtOAc in pet ether) to give 4-chloro-3-methyl-5,7-dihydrofuro[3,4-b]pyridine. ¹H NMR (400 MHz, chloroform-d) δ ppm 8.31 (s, 1H), 5.18 (s, 2H), 5.11 (s, 2H), 2.37 (s, 3H).

### 3-Iodo-4-methyl-1-(3-methyloxetan-3-yl)-1H-pyrazole (Intermediate A-24)

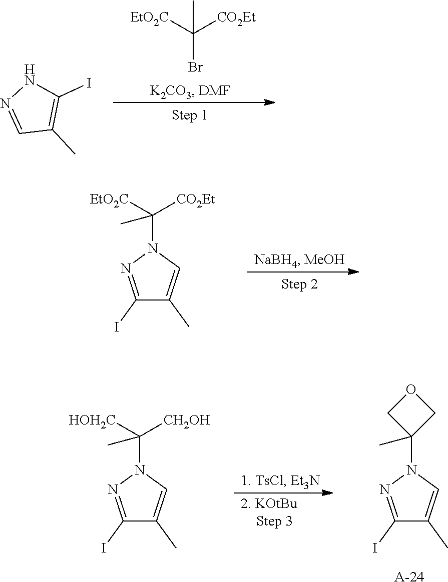

Step 1. To a 100 mL glass tube was added 5-iodo-4-methyl-1H-pyrazole (5 g, 24.04 mmol), potassium carbonate (6.64 g, 48.1 mmol), diethyl 2-bromo-2-methylmalonate (7.30 g, 28.8 mmol), and DMF (30 mL). The reaction mixture was stirred at 80° C. for 12 h. The reaction mixture was diluted with H₂O (100 mL) and extracted with EtOAc (2×50 mL). The combined organic extracts were washed with H₂O (2.25 mL) and dried over Na₂SO₄. The solution was filtered, concentrated in vacuo, and purified by chromatography eluting with a gradient of 0% to 30% EtOAc in hexanes, to provide diethyl 2-(5-iodo-4-methyl-1H-pyrazol-1-yl)-2-methylmalonate. m/z (ESI): 381.0 (M+H)⁺.

Step 2. To a 100 ml, round-bottomed flask was added diethyl 2-(5-iodo-4-methyl-1H-pyrazol-1-yl)-2-methylmalonate (8 g, 21.04 mmol) and MeOH (80 mL) The reaction mixture was cooled to 0° C., and sodium borohydride (1.194 g, 31.6 mmol) was added slowly. The reaction mixture was stirred at rt for 4 h and quenched by the addition of 1N HCl. The reaction mixture was concentrated and purified by reverse phase HPLC to give 2-(5-iodo-4-methyl-1H-pyrazol-1-yl)-2-methylpropane-1,3-diol. m/z (ESI): 296.9 (M+H)⁺.

Step 3. To a 50 mL round-bottomed flask was added 2-(5-iodo-4-methyl-1H-pyrazol-1-yl)-2-methylpropane-1,3-diol (2.2 g, 7.43 mmol), TEA (2.59 mL, 18.57 mmol), and THF (20 mL). The reaction mixture was cooled to 0° C., and TsCl (1.558 g, 8.17 mmol) was added slowly. The reaction mixture was stirred at rt for 3 h. The crude material was absorbed onto a plug of silica gel and purified by chromatography eluting with a gradient of 0% to 60% EtOAc in hexanes, to provide 2-(5-iodo-4-methyl-1H-pyrazol-1-yl)-2-methylpropane-1,3-diol and 3-hydroxy-2-(5-iodo-4-methyl-1H-pyrazol-1-yl)-2-methylpropyl 4-methylbenzenesulfonate.

To a 25-mL round-bottomed flask was added 3-hydroxy-2-(5-iodo-4-methyl-1H-pyrazol-1-yl)-2-methylpropyl 4-methylbenzenesulfonate (1 g, 2.221 mmol) and potassium tert-butoxide (0.748 g, 6.66 mmol) and THF (10 mL). The reaction mixture was stirred at rt for 3 h and concentrated under vacuum. The crude material was absorbed onto a plug of silica gel and purified by chromatography, eluting with a gradient of 0% to 40% EtOAc in hexanes, to provide 5-iodo-4-methyl-1-(3-methyloxetan-3-yl)-1H-pyrazole. m/z (ESI): 279.0 (M+H)⁺.

### (S)-3-Iodo-4-methyl-1-(tetrahydrofuran-3-yl)-1H-pyrazole (Intermediate A-25)

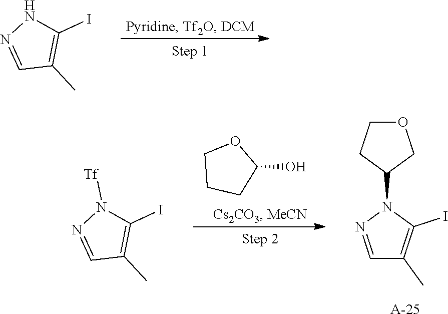

Step 1. To a stirred suspension of 5-iodo-4-methyl-1H-pyrazole (650 g, 3125 mmol) in DCM (6500 mL) was added pyridine (379 mL, 4687 mmol) at rt under nitrogen atmosphere and the reaction mixture was cooled to 0° C. To the reaction mixture was added trifluoromethanesulfonic anhydride (634 mL, 3750 mmol) dropwise and the reaction mixture was stirred at rt for 30 min. The reaction mixture was quenched with sat aq. NH₄Cl solution (2500 mL) and the layers were separated. The aqueous layer was extracted with DCM (2500 mL) and the organic extract was washed with brine (3000 mL), dried over anhydrous Na₂SO₄, and concentrated under vacuum to afford 5-iodo-4-methyl-1-((trifluoromethyl)sulfonyl)-1H-pyrazole. m/z (ESI): 213.0 (M+H)⁺. ¹H NMR (401 MHz, DMSO-d₆): δ (ppm) 8.45 (s, 1H), 2.02 (s, 3H).

Step 2. To a stirred solution of 5-iodo-4-methyl-1-((trifluoromethyl)sulfonyl)-1H-pyrazole (1055 g, 3102 mmol) in ACN (10.6 L) was added (R)-tetrahydrofuran-3-ol (301 g, 3413 mmol) and Cs₂CO₃ (2527 g, 7756 mmol) at 0° C., and the resulting reaction mixture was stirred for 16 h at rt. The reaction mixture was diluted with H₂O (5000 mL), and extracted with EtOAc (4000×2 mL). The combined organic extracts were dried over Na₂SO₄, the solution was filtered, and concentrated in vacuo. The crude material was absorbed onto a plug of silica gel and purified by column chromatography through a silica gel column, eluting with a gradient of 0% to 8% EtOAc in hexanes, to provide (S)-5-iodo-4-methyl-1-(tetrahydrofuran-3-yl)-1H-pyrazole and after eluting with a gradient of 8% to 15% EtOAc in hexanes to provide (S)-3-iodo-4-methyl-1-(tetrahydrofuran-3-yl)-1H-pyrazole. m/z (ESI): 279.1 (M+H)⁺. ¹H NMR (401 MHz, DMSO-d₆): δ 7.45 (s, 1H), 5.07-5.03 (m, 1H), 4.04-3.93 (m, 2H), 3.86-3.81 (m, 1H), 3.78-3.75 (m, 1H), 2.34-2.23 (m, 2H), 1.95 (s, 3H).

The intermediates in the table below were prepared in a fashion similar to that described above.

| | | | m/z (ESI): |
|---|---|---|---|
| Int. # | Chemical Structure | Name | (M + H)⁺ |
| A-26 | 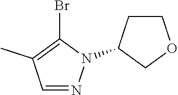 | (R)-5-bromo-4- methyl-1- (tetrahydrofuran- 3-yl)-1H-pyrazole | 231.0 |
| A-27 | 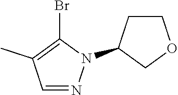 | (S)-5-bromo-4- methyl-1- (tetrahydrofuran-3- yl)-1H-pyrazole | 231.0 |
| A-28 | 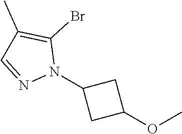 | cis-5-bromo-1-(3- methoxycyclobutyl)-4- methyl-1H-pyrazole | 246.1 |

### (1-(1-Methoxycyclopropyl)-4-methyl-1H-pyrazol-5-yl)boronic Acid (Intermediate A-29)

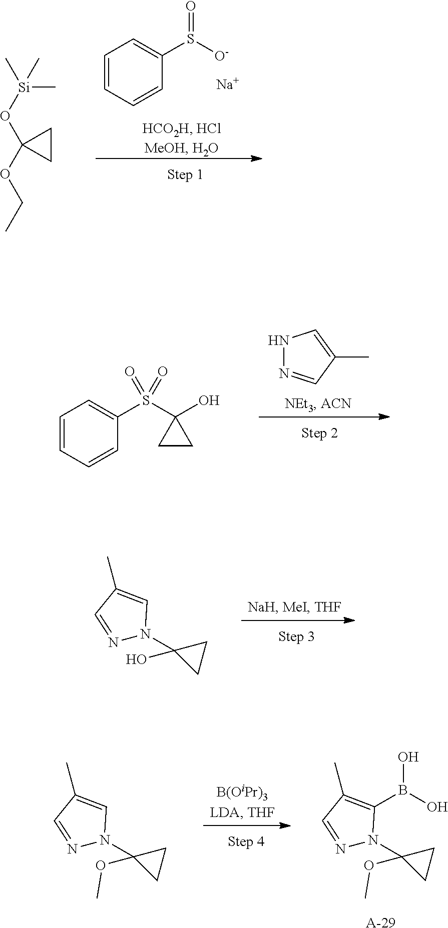

Step 1. To a solution of (1-ethoxycyclopropoxy)trimethylsilane (10.00 mL, 57.4 mmol, Sigma-Aldrich. Inc.) in MeOH (28.7 mL) was added conc. HCl (1.8 μL, 0.057 mmol, Sigma-Aldrich, Inc.) and the reaction mixture was stirred at rt for 10 min. To the reaction mixture was added H₂O (57.3 mL), sodium benzenesulfinate (18.83 g, 115 mmol, Combi-Blocks. Inc.), and formic acid 95-97% (21.64 mL, 574 mmol, Sigma-Aldrich, Inc.) and the reaction mixture was stirred at rt over 2 days. The reaction mixture was diluted with H₂O and extracted with DCM. The organic extracts were washed with brine. dried over Na₂SO₄, filtered, and concentrated in vacuo to afford 1-(phenylsulfonyl)cyclopropan-1-ol that was used without further purification. m/z (ESI): 199.0 (M+Na)⁺.

Step 2. To a flask was added 4-methyl-1H-pyrazole (4.64 g, 56.5 mmol, Ambeed. Inc.) and 1-(phenylsulfonyl)cyclopropan-1-ol (11.8 g, 59.5 mmol) and the reaction vessel was evacuated and backfilled with N₂. To the reaction mixture was added ACN (119 mL) and TEA (8.37 mL, 59.5 mmol) and the reaction mixture was stirred at rt for 3 h. The reaction mixture was concentrated and diluted with sat. aq. NH₄Cl and EtOAc. The organic phase was separated and the aqueous phase was extracted with EtOAc (×2). The combined organic extracts were dried over Na₂SO₄, filtered, and concentrated in vacuo. The crude mixture was dissolved in MeCN and conc NH₄OH (aq) solution was added to the solution until pH=9. Then, the aqueous phase was extracted with DCM (×2). The combined organic extracts were washed with brine, dried over Na₂SO₄, filtered, and concentrated in vacuo. The crude material was filtered through a plug of silica and concentrated under vacuum to afford 1-(4-methyl-1H-pyrazol-1-yl)cyclopropan-1-ol. m/z (ESI): 139.1 (M+H)⁺.

Step 3. To a suspension of sodium hydride (60% dispersion in mineral oil, 4.56 g, 114 mmol, TCI America) in THF (15 mL) at 0° C. was added a solution of 1-(4-methyl-1H-pyrazol-1-yl)cyclopropan-1-ol (7.88 g, 57.0 mmol) in THF (8 mL) dropwise and the reaction mixture was stirred at this temperature until bubbling ceased. To the reaction mixture was added iodomethane (7.13 mL, 114 mmol, Sigma-Aldrich, Inc.) and the reaction mixture was warmed to rt and stirred for 18 h. The reaction mixture was carefully quenched by the slow addition of H₂O and was extracted with EtOAc. The organic extracts were washed with brine, dried over Na₂SO₄, filtered, and concentrated in vacuo. The crude material was purified by passing thru a silica gel column and washing the column with EtOAc (2×250 mL) to afford 1-(1-methoxycyclopropyl)-4-methyl-1H-pyrazole. m/z (ESI): 153.2 (M+H)⁺.

Step 4. To a solution of 1-(1-methoxycyclopropyl)-4-methyl-1H-pyrazole (2.12 g, 13.93 nmol) in THF (10.65 mL) at −78° C., was added a solution of LDA (2 M in THF, 10.45 mL, 20.89 mmol, Sigma-Aldrich. Inc.) and the reaction mixture was stirred for 30 min. To the reaction mixture was added triisopropyl borate (4.85 mL, 20.89 mmol, Sigma-Aldrich, Inc.) and the reaction mixture was stirred for 1 h allowing to warmup to rt. The reaction mixture was quenched by the addition of sat. aq. NH₄Cl and extracted with EtOAc. The organic extracts were filtered through a plug of silica and concentrated in vacuo to provide (1-(1-methoxycyclopropyl)-4-methyl-1H-pyrazol-5-yl)boronic acid which was used without further purification. m/z (ESI): 197.2 (M+H)⁺.

The intermediates in the table below were prepared in a fashion similar to that described above.

| | | | m/z (ESI): |
|---|---|---|---|
| Int. # | Chemical Structure | Name | (M + H)⁺ |
| A-30 | 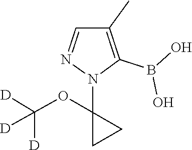 [SMILES: [2H]C([2H])([2H])OC1(n2ncc(C)c2B(O)O)CC1] | (1-(1-(methoxy-d3)cyclopropyl)-4-methyl-1H- pyrazol-5-yl)boronic acid | 200.2 |
| A-31 | 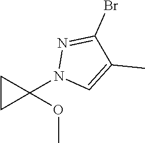 | 3-bromo-1-(1-methoxycyclopropyl)-4-methyl- 1H-pyrazole | 231.0 |

### (1-(1-(Methoxymethyl)cyclopropyl)-4-methyl-1H-pyrazol-5-yl)boronic Acid (Intermediate A-32)

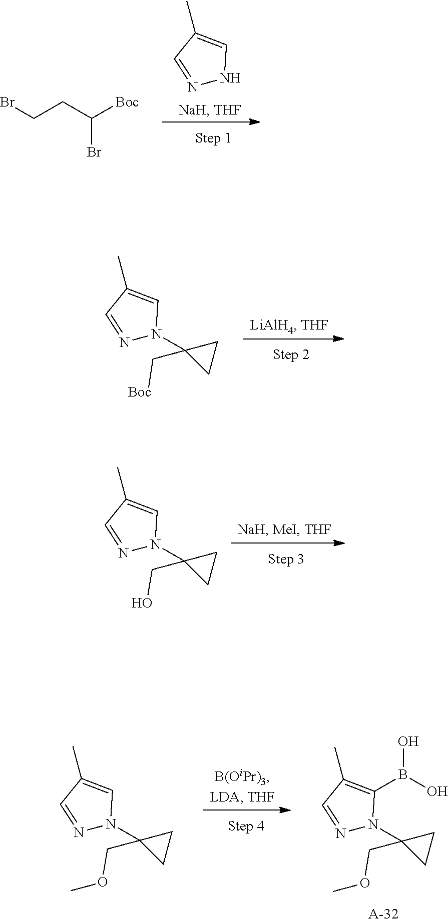

Step 1. To a suspension of sodium hydride (60% dispersion in mineral oil, 1.072 g, 26.8 mmol, TCI) in THF (20 mL) at 0° C., was added a solution of 4-methyl-1h-pyrazole (1 g, 12.18 mmol, Ambeed, Inc.) in THF (5 mL) dropwise and the reaction mixture was stirred at this temperature until bubbling ceased. To the reaction mixture was added tert-butyl 2,4-dibromobutyrate (3.68 g, 12.18 mmol, Combi-Blocks, Inc.) and the reaction mixture was warmed to rt and stirred for 5 h. The reaction mixture was carefully quenched by the slow addition of H₂O and was extracted with EtOAc. The organic extracts were washed sat. aq. NH₄Cl, then with brine, dried over a plug of silica, and concentrated in vacuo to give the crude tert-butyl 1-(4-methyl-1H-pyrazol-1-yl)cyclopropane-1-carboxylate. The material was used in the next step without further purification. m/z (ESI): 223.0 (M+H)⁺.

Step 2. To a solution of tert-butyl 1-(4-methyl-1H-pyrazol-1-yl)cyclopropane-1-carboxylate (2.573 g, 11.58 mmol) in THF (23.15 mL) at 0° C., was added lithium aluminum hydride (1.0 M in THF, 12.73 mL, 12.73 mmol, Sigma-Aldrich Corporation) dropwise and the reaction mixture was stirred at this temperature for 1 h. The reaction mixture was quenched at 0° C. via the slow addition of H₂O until bubbling stopped. The reaction mixture was then diluted with 1 M NaOH and stirred at rt for 15 min. The mixture was filtered and diluted with EtOAc. The organic phase was separated, and the aqueous phase was extracted with EtOAc. The combined organic extracts were washed with brine, dried over Na₂SO₄, then filtered through a small plug of silica to give (1-(4-methyl-1H-pyrazol-1-yl)cyclopropyl)methanol. m/z (ESI): 153.2 (M+H)⁺.

Step 3. To a suspension of NaH (60% dispersion in mineral oil, 0.526 g, 13.14 mmol, TCI America) in THF (10 mL) at 0° C., was added a solution of (1-(4-methyl-1H-pyrazol-1-yl)cyclopropyl)methanol (1 g, 6.57 mmol) in THF (3 mL) dropwise and the reaction mixture was stirred at this temperature until bubbling ceased. To the reaction mixture was added iodomethane (0.822 mL, 13.14 mmol, Sigma-Aldrich. Inc.) and the reaction mixture was warmed to rt and stirred for 18 h. The reaction mixture was carefully quenched by the slow addition of H₂O and extracted with EtOAc. The organic extracts were washed with brine, dried over Na₂SO₄, filtered through a plug of silica, and concentrated in vacuo to give 1-(1-(methoxymethyl)cyclopropyl)-4-methyl-1H-pyrazole that was used without further purification. m/z (ESI): 167.2 (M+H)⁺.

Step 4. To a solution of 1-(1-(methoxymethyl)cyclopropyl)-4-methyl-1H-pyrazole (0.8853 g, 5.33 mmol) in THF (10.65 mL) at −78° C. was added a solution of LDA (1.0 M in THF, 7.99 mL, 7.9) mmol, Sigma-Aldrich. Inc.) and the reaction mixture was stirred for 30 min. To the reaction mixture was added triisopropylborate (1.855 mL, 7.99 mmol, Sigma-Aldrich, Inc.) and the reaction mixture was stirred for 1 h allowing to warm up to rt. The reaction mixture was quenched by the addition of sat. aq. NH₄Cl and extracted with EtOAc. The organic extracts were filtered through a small plug of silica and concentrated in vacuo. The crude material was purified by chromatography through a Redi-Sep pre-packed silica gel column (24 g), eluting with a gradient of 0% to 100°/o EtOAc in heptane, to provide (1-(1-(methoxymethyl)cyclopropyl)-4-methyl-1H-pyrazol-5-yl)boronic acid. m/z (ESI): 211.15 (M+H)⁺.

### 3-Bromo-4-chloro-2-(3-methoxyoxetan-3-yl)pyridine (Intermediate A-33)

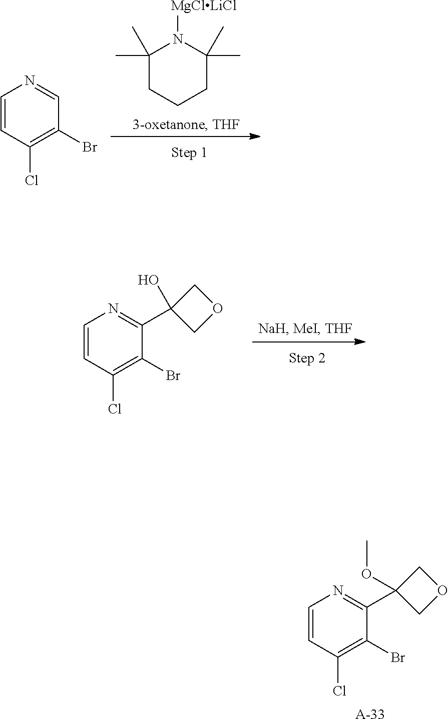

Step 1. To a solution of 3-bromo-4-chloropyridine (1.5 g, 7.79 mmol, Combi-Blocks, Inc.) in THF (15 mL) at −45° C. was added 2,2,6,6-tetramethylpipendinylmagnesium chloride lithium chloride complex solution (1.0 M in THF/toluene) (11.69 mL, 11.69 mmol, Sigma-Aldrich, Inc.) and the reaction mixture was stirred at this temperature for 30 min. To the reaction mixture was added 3-oxetanone (0.562 mL, 7.79 mmol, Combi-Blocks. Inc.) and the reaction mixture was stirred at −45° C. for 10 min, then warmed to rt and stirred for an additional 30 min. The reaction mixture was quenched by the addition of sat. aq. NH₄Cl and extracted with EtOAc. The combined organic extracts were washed with brine, dried over Na₁SO₄, filtered, and concentrated in vacuo. The crude material was purified b) chromatography through a Redi-Sep pre-packed silica gel column (24 g), eluting with a gradient of 0% to 20% EtOAc in heptane, to provide 3-(3-bromo-4-chloropyridin-2-yl)oxetan-3-ol. m/z (ESI): 263.8 (M+H)⁺.

Step 2. To a glass vial was added sodium hydride (60% dispersion in mineral oil, 0.441 g, 11.02 mmol, TCI America) and the reaction vessel was evacuated and backfilled with N₂. To the reaction mixture was added THF (11.02 mL) and the reaction mixture was cooled to 0° C. in an ice bath. To the reaction mixture was added a solution of 3-(3-bromo-4-chloropyridin-2-yl)oxetan-3-ol (1.457 g, 5.51 nmol) in THF (5 mL) dropwise and the reaction mixture was stirred for 15 min or until bubbling ceased. To the reaction mixture was added iodomethane (0.686 mL, 11.02 mmol, Sigma-Aldrich, Inc.) and the reaction mixture was stirred at it 18 h. The reaction mixture was quenched at 0° C. by the careful dropwise addition of H₂O and extracted with EtOAc. The organic extracts were washed with brine, dried over anhydrous Na₂SO₄, filtered, and concentrated in vacuo. The crude material was purified by chromatography through a Redi-Sep pre-packed silica gel column (12 g), eluting with a gradient of 0% to 20% EtOAc in heptane, to provide 3-bromo-4-chloro-2-(3-methoxyoxetan-3-yl)pyridine. m/z (ESI): 278.0 (M+H)⁺.

### Tert-Butyl 3-methyl-4-(((trifluoromethyl)sulfonyl)oxy)-3,6-dihydropyridine-1(2H)-carboxylate (Intermediate A-34)

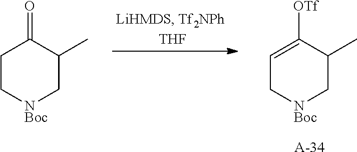

To a solution of LiHMDS (1.0 M in THF, 46.9 mL, 46.9 mmol) in THF (30 mL) at −78° C., was added dropwise tert-butyl 3-methyl-4-oxopiperidine-1-carboxylate (45 g, 23.44 mmol) in THF (20 mL). The mixture was stirred for 2 h at −70° C., and at this temperature, 1,1,1-trifluoro-N-phenyl-N-((trifluoromethyl)sulfonyl)methanesulfonamide (12.56 g, 35.2 mmol in THF (50 mL) was added dropwise. The mixture was warmed to 20° C., and stirred for 4 h. The reaction was quenched with aq. NH₄Cl (50 mL) and extracted with EtOAc (3×50 mL). The combined organic extracts were dried over Na₂SO₄ and concentrated. The residue was purified by chromatography (eluting with a gradient of 0% to 10% EtOAc in pet. ether) to afford tert-butyl 3-methyl-4-(((trifluoromethyl)sulfonyl)oxy)-3,6-dihydropyridine-1(2H)-carboxylate. ¹H NMR (400 MHz, CHLOROFORM-d) δ=5.74 (s, 1H), 4.34-3.91 (m, 2H), 3.76-3.51 (m, 1H), 3.49-3.31 (m, 1H), 2.64 (s, 1H), 1.49 (s, 10H), 1.15 (d, J=20.0 Hz, 3H).

The intermediates in the table below were prepared in a fashion similar to that described above.

| | | | m/z |
|---|---|---|---|
| | | | (ESI): |
| | | | (M + |
| Int. # | Chemical Structure | Name | H − 57)⁺ |
| A-35 | 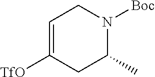 [SMILES: C[C@@H]1CC(OS(=O)(=O)C(F)(F)F)=CCN1C(=O)OC(C)(C)C] | tert-butyl (R)-2-methyl-4- (((trifluoromethyl) sulfonyl)oxy)-3,6- dihydropyridine- 1(2H)-carboxylate | 289.9 |
| A-36 | 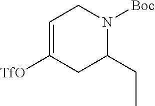 [SMILES: CCC1CC(OS(=O)(=O)C(F)(F)F)=CCN1C(=O)OC(C)(C)C] | tert-butyl 2-ethyl-4- (((trifluoromethyl) sulfonyl)oxy)-3,6- dihydropyridine- 1(2H)-carboxylate | 304.0 |

### 3-Chloro-2-(1-methoxycyclopropyl)-4-methylpyridine (Intermediate A-37)

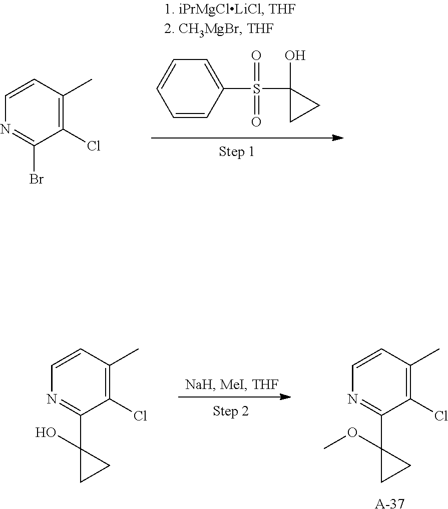

Step 1. To a 0° C. solution of 2-bromo-3-chloro-4-methylpyridine (5.76 g, 27.9 mmol, Combi-Blocks, Inc.) in THF (25 mL) was added isopropylmagnesium chloride. lithium chloride complex (1.3 M in THF, 21.46 mL, 27.9 mmol, Sigma-Aldrich. Inc.) and the reaction mixture was stirred at this temperature for 1 h. To a separate reaction vial was added 1-(phenylsulfonyl)cyclopropan-1-ol (5.53 g, 27.9 mmol), THF (30 mL) and the reaction mixture was cooled to −78° C. To the reaction mixture was added methylmagnesium bromide solution (3.0 M in diethyl ether, 9.30 mL, 27.9 mmol, Sigma-Aldrich, Inc.) followed by the Grignard solution prepared above. The reaction mixture was stirred at rt for 18 h. The reaction mixture was quenched by the addition of 10 M HCl and extracted with EtOAc. The aqueous phase was then washed with 1.0 M NaOH and extracted once more with EtOAc. The combined organic extracts were washed with brine, dried over Na₂SO₄, filtered, and concentrated in vacuo. The crude material was purified by silica gel chromatography, eluting with a gradient of 0% to 40% EtOAc in heptane, followed by 0% to 60% EtOAc/EtOH (3:1) in heptane to provide 1-(3-chloro-4-methylpyridin-2-yl)cyclopropan-1-ol. m/z (ESI): 184.2 (M+H)⁺

Step 2. To a 0° C. suspension of sodium hydride (60% dispersion in mineral oil, 0.650 g, 16.26 mmol, TCI America) in THF (10 mL) was added a solution of 1-(3-chloro-4-methylpyridin-2-yl)cyclopropan-1-ol (1.99 g, 10.84 mmol) in THF (10 mL) dropwise and the reaction mixture was stirred at this temperature until bubbling ceased. To the reaction mixture was added iodomethane (1.016 mL, 16.26 mmol, Sigma-Aldrich. Inc.) and the reaction mixture was warmed to rt and stirred for 1 h. The reaction mixture was carefully quenched by the slow addition of H₂O and was cataracted with EtOAc. The organic extracts were washed with brine, dried over Na₂SO₄, filtered, and concentrated in vacuo. The crude material was purified by chromatography through a Redi-Sep pre-packed silica gel column, eluting with a gradient of 0% to 40% EtOAc in heptane, to provide 3-chloro-2-(methoxycyclopropyl)-4-methylpyridine). m/z (ESI): 198.0 (M+H)⁺.

### 6-(4-Bromo-5-methylisoxazol-3-yl)-2-oxa-6-azaspiro[3.3]heptane (Intermediate A-38)

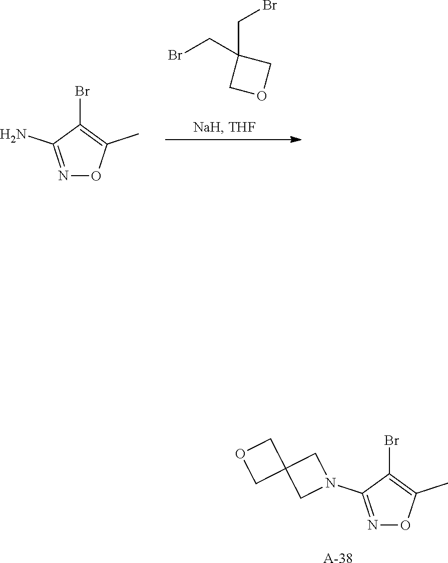

To a suspension of sodium hydride (60% dispersion in mineral oil, 542 mg, 13.56 mmol, Oakwood Products, Inc.) in THF (8 mL) at 0° C. was added a solution of 3-amino-4-bromo-5-methylisoxazole (800 mg, 4.52 mmol, Combi-Blocks, Inc.) in THF (2 mL) and the reaction mixture was stirred at this temperature for 30 min. To the reaction mixture was added a solution of 3,3-bis(bromomethyl)octane (1102 mg, 4.52 mmol, Combi-Blocks, Inc.) in THF (2 mL) and the reaction mixture was stirred at rt for 16 h. The reaction was quenched by the slow addition of H₂O and extracted with EtOAc. The organic extract was a washed with brine, dried over Na₂SO₄, filtered, and concentrated in vacuo. The crude material was purified by chromatography through a Redi-Sep pre-packed silica gel column, eluting with a gradient of 0% to 80% EtOAc/EtOH (3:1) in heptane, to provide 6-(4-bromo-5-methylisoxazol-3-yl)-2-oxa-6-azaspiro[3.3]heptane. m/z (ESI): 259.0 (M+H)⁺.

### 5-Bromo-4-methyl-1-(3-methyloxetan-3-yl)-1H-pyrazole (Intermediate A-39)

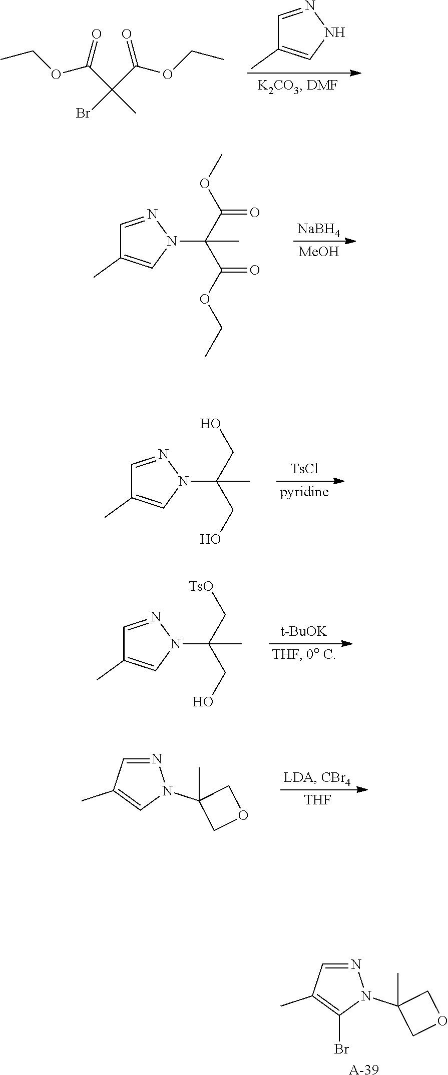

Step 1. A RBF was charged 4-methyl-1H-pyrazole (1981 mg, 24.13 mmol, Ambeed), potassium carbonate, and DMF (50 mL) followed b diethyl 2-bromo-2-methylmalonate (7328 mg, 29.0 mmol, Cambridge Isotope Laboratories, Inc.). The reaction was stirred at 80° C. After 16 h, the reaction mixture was partitioned between EtOAc and water. The aqueous layer was extracted with EtOAc (3×25 mL). The combined organic extracts were washed with water, brine, and concentrated in vacuo to provide diethyl 2-methyl-2-(4-methyl-1H-pyrazol-1-yl)malonate. m/z (ESI): 255.1 (M+H)⁺.

Step 2. To a solution of diethyl 2-methyl-2-(4-methyl-1H-pyrazol-1-yl)malonate (4.9 g, 19.27 mmol) in MeOH (70 mL) at 0° C. was added sodium borohydride (1.458 g, 38.5 mmol, Sigma-Aldrich, Inc.) in 5 portions over 15 mm. After 4 h, the reaction mixture was quenched with water and then partitioned between EtOAc and water. The aqueous layer was extracted with EtOAc:EtOH (3:1) (9×25 mL). The combined organic extracts were washed with brine and concentrated in vacuo. The material was adsorbed onto a plug of silica gel and chromatographed, eluting with 0-100% EtOAc:EtOH (3:1) in heptane, to provide 2-methyl-2-(4-methyl-1H-pyrazol-1-yl)propane-1,3-diol. m/z (ESI): 171.1 (M+H)⁺.

Step 3. To a solution of 2-methyl-2-(4-methyl-1H-pyrazol-1-yl)propane-1,3-diol (4.3 g, 25.3 mmol) and pyridine (50 mL) was added para-toluenesulfonyl chloride (6.02 g, 31.6 mmol, Sigma-Aldrich, Inc.) in 4 portions over 10 mm. After 16 h, an additional 800 mg of TsCl was added. After a further 4 h, the reaction mixture was partitioned between EtOAc and water. The aqueous layer was extracted with EtOAc (4×25 mL). The combined organic extracts were concentrated in vacuo and adsorbed onto a plug of silica gel and chromatographed, eluting with 0-100% EtOAc:EtOH (3.1) in heptane, to provide 3-hydroxy-2-methyl-2-(4-methyl-1H-pyrazol-1-yl)propyl 4-methylbenzenesulfonate. m/z (ESI): 325.0 (M+H)⁺.

Step 4. To a solution of 3-hydroxy-2-methyl-2-(4-methyl-1H-pyrazol-1-yl)propyl 4-methylbenzenesulfonate (3.1 g, 9.56 mmol) in 2-methyltetrahydrofuran (40 mL) at 0° C. was added potassium tert-butoxide (1.25 g, 11.14 nmol, Sigma-Aldrich, Inc.). The solution was stirred and allowed to warm up to rt. After 16 h, the reaction mixture was treated with an additional 400 mg of KOtBu and stirred for a further 2 h. The reaction mixture was partitioned between EtOAc and water. The aqueous layer was extracted with EtOAc (3×20 mL). The combined organic extracts were concentrated in vacuo and adsorbed onto a plug of silica gel and chromatographed, eluting with 0-100% EtOAc:EtOH (3:1) in heptane, to provide 4-methyl-1-(3-methyloxetan-3-yl)-1H-pyrazole. m/z (ESI): 153.1 (M+H)⁺.

Step 5. To a solution of 4-methyl-1-(3-methyloxetan-3-yl)-1H-pyrazole (1.2 g, 7.88 mmol) in 2-methyltetrahydrofuran (18 mL) at −78° C. was added LDA (1 M in THF/hexanes. 15.77 mL, 15.77 mmol, Sigma-Aldrich, Inc.) dropwise over 5 min. The reaction was stirred at −78° C. for 1 h, then treated dropwise with a solution of carbon tetrabromide (3.92 g, 11.83 mmol, Combi-Blocks, Inc.) in THF (10 mL) over 4 min. The reaction mixture was allowed to warm to rt After 1 h, the reaction mixture was partitioned between EtOAc and H₂O. The aqueous layer was extracted with EtOAc (6×15 mL). The combined organic extracts were concentrated in vacuo and adsorbed onto a plug of silica gel and chromatographed, eluting with 0-100% EtOAc in heptane, to provide 5-bromo-4-methyl-1-(3-methyloxetan-3-yl)-1H-pyrazole. m/z (ESI): 231.0, 232.9 (M+H)⁺.

### 1-(1-Methoxy-2-methylpropan-2-yl)-4-methyl-5-(4,4,5,5-tetramethyl-1,3,2-dioxaborolan-2-yl)-1H-pyrazole (Intermediate A-40)

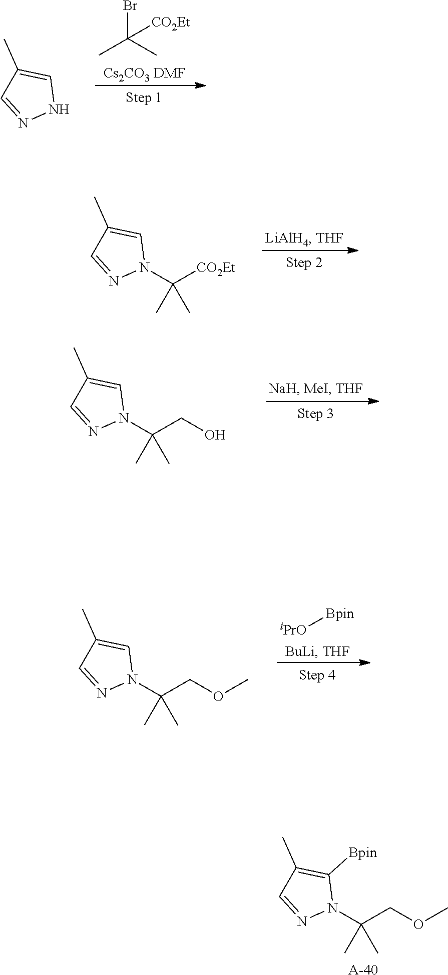

Step 1. To a solution of 4-methyl-1H-pyrazole (4 g, 48.7 mmol, Ambeed, Inc.) and DMF (100 mL) was added Cs₂CO₃ (18.53 g, 56.9 mmol), followed by ethyl 2-bromo-2-methylpropanoate (10.45 g, 7.87 mL, 53.6 mmol, Sigma-Aldrich, Inc.). The solution was stirred at 60° C. for 2 h. The resulting product underwent workup to provide crude ethyl 2-methyl-2-(4-methyl-1H-pyrazol-1-yl)propanoate, which was used as is. m/z (ESI): 197.1 (M+H)⁺.

Step 2. To a 0° C. solution of ethyl 2-methyl-2-(4-methyl-1H-pyrazol-1-yl)propanoate (4 g, 12.23 mmol) in THF (20 mL) was treated dropwise with LAH (2 M in THF, 6.73 mL, 13.45 mmol, Sigma-Aldrich. Inc.), and stirred for 30 min. The reaction was cautiously quenched with H₂O, then diluted with 1N NaOH (10 mL). After stirring for 15 min, the reaction was filtered, the filtrate was diluted with brine, and the extracts were separated and concentrated in vacuo. The crude product was eluted through a short plug of silica gel using EtOAc and the filtrate was concentrated in vacuo to give 2-methyl-2-(4-methyl-1H-pyrazol-1-yl)propan-1-ol. m/z (ESI): 155.1 (M+H)⁺.

Step 3. To a solution of 2-methyl-2-(4-methyl-1H-pyrazol-1-yl)propan-1-ol (1.886 g, 12.23 mmol) in THF (60 mL) at 0° C. was added sodium hydride)(60 wt % in mineral oil, 0.734 g, 18.34 mmol, TC America) in two portions. After stirring for 30 min, iodomethane (1.05 mL, 16.87 mmol, Sigma-Aldrich. Inc.) was added, and stirred for 45 min. The reaction was removed from the cooling bath and allowed to stir for an additional 30 min, then quenched with aqueous satd NH₄Cl. The reaction was partitioned between EtOAc and H₂O. The aqueous layer was extracted with EtOAc (3×15 mL). The combined organic extracts n ere concentrated in vacuo and adsorbed onto a plug of silica gel and chromatographed, eluting with 0-50% EtOAc EtOH (3:1) in heptane, to provide 1-(1-methoxy-2-methylpropan-2-yl)-4-methyl-1H-pyrazole. m/z (ESI): 169.2 (M+H)⁺

Step 4. To a 0° C. solution of 1-(1-methoxy-2-methylpropan-2-yl)-4-methyl-1H-pyrazole (1.234 g, 7.33 mmol) in THF (25 mL) was added n-BuLi, 2.5 M in hexanes (8.80 mL, 22.00 mmol, Sigma-Aldrich, Inc.) dropwise over 1 min. After stirring for 30 min at 0° C., the solution was cooled to −78° C., and 2-isopropoxy-4,4,5,5-tetramethyl-1,3,2-dioxaborolane (4.49 mL, 22.00 mmol, Sigma-Aldrich Corporation) was added dropwise. The reaction mixture was allowed to warm to rt. The reaction mixture was quenched with H₂O and partitioned between EtOAc and H₂O. The aqueous layer was extracted with EtOAc (4×25 mL). The combined organic extracts were washed with brine and concentrated in vacuo. The crude material was adsorbed onto a plug of silica gel and chromatographed, eluting with 0-75% EtOAc in heptane, to provide 1-(1-methoxy-2-methylpropan-2-yl)-4-methyl-5-(4,4,5,5-tetramethyl-1,3,2-dioxaborolan-2-yl)-1H-pyrazole. m/z (ESI): 295.1 (M+H)⁺.

### 5-Bromo-4-(3-fluorooxetan-3-yl)thiazole (Intermediate A-41)

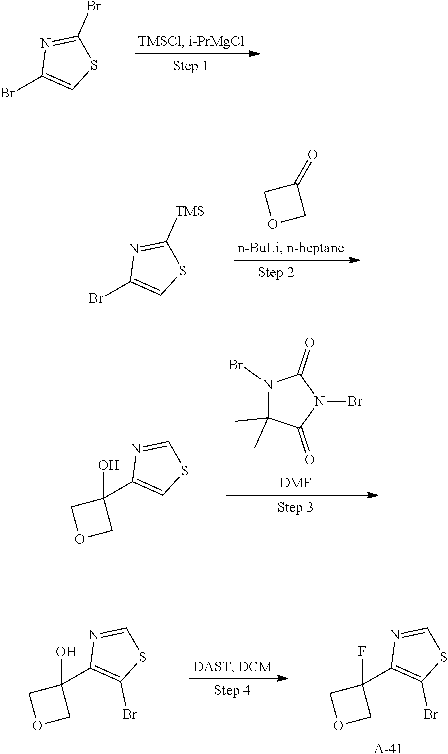

Step 1. To a solution of 2,4-dibromothiazole (100 g, 412 mmol) in THF (1600 mL) at 0° C. t as added isopropylmagnesium chloride (2 M in THF, 226 mL, 453 mmol) dropwise. The reaction mixture was stirred at 0° C. for 1 h, and chlorotrimethylsilane (49.2 g, 453 mmol) was added dropwise at 0° C. The resulting mixture was stirred at 0° C. for 3 h. The reaction mixture was quenched by the addition of H₂O (1.6 L), and extracted with EtOAc (3×1000 mL). The combined organic extracts were dried over Na₂SO₄, filtered, and concentrated under reduced pressure. The residue was purified by column chromatography (SiO₂, pet ether/EtOAc=100/1 to 80/1) to give 4-bromo-2-(trimethylsilyl)thiazole.

Step 2. To a vessel was added 4-bromo-2-(trimethylsilyl)thiazole (30 g, 127 mmol), n-heptane (900 mL), and n-BuLi (2.5 M in hexanes, 102 mL, 254 mmol) dropwise at −70° C. The mixture was stirred at −70° C. for 1 h, and then oxetan-3-one (27.5 g, 381 mmol) was added dropwise at −70° C. The resulting mixture was stirred at −70° C. for 3 h. The reaction mixture was quenched by addition of H₂O (2 L), and extracted with EtOAc (3×1 L). The combined organic extracts were dried over Na₂SO₄, filtered, and concentrated under reduced pressure. The residue was purified by column chromatography (SiO₂, pet ether/EtOAc=100/1 to 1/1) to give 3-(thiazol-4-yl) oxetan-3-ol.

Step 3. To a solution of 3-(thiazol-4-yl) oxetan-3-ol (20 g, 127 mmol) in DMF (400 mL) at 0° C. was added 1,3-dibromo-5,5-dimethylimidazolidine-2,4-dione (54.6 g, 191 mmol) slowly. The mixture was stirred at 20° C. for 2 h. The reaction mixture was quenched by addition of H₂O (1 L), and extracted with EtOAc (3×500 mL). The combined organic extracts were washed with brine (3×300 mL), dried over Na₂SO₄, filtered, and concentrated under reduced pressure. The residue was purified by column chromatography (SiO₂. pet ether/EtOAc=10/1 to 3/1) to give 3-(5-bromothiazol-4-yl) oxetan-3-ol. m/z (ESI): 235.9 (M+H)⁺. ¹H NMR (400 MHz, CHLOROFORM-d) δ ppm 8.73 (s, 1H), 5.30 (d, J=8.0 Hz, 2H), 4.95-4.92 (m, 2H).

Step 4. To a solution of 3-(5-bromothiazol-4-yl) oxetan-3-ol (19 g, 80 mmol) in DCM (380 mL) at 0° C. was added DAST (21.27 mL, 161 mmol) dropwise. The mixture was stirred at 0° C. for 1 h. The reaction mixture was adjusted to pH=7-8 with sat. NaHCO₃ and extracted with DCM (3×400 mL). The combined organic extracts were dried over Na₂SO₄, filtered, and concentrated under reduced pressure. The residue was purified by column chromatography (SiO₂, pet ether/EtOAc=20/1 to 10/1) to give 5-bromo-4-(3-fluorooxetan-3-yl)thiazole. m/z (ESI): 237.9 (M+H)⁺. ¹H NMR (400 MHz, CHLOROFORM-d) δ ppm 8.76 (s, 1H), 5.40-5.20 (m, 2H), 5.20-5.00 (m, 2H).

### 6-(3-Bromo-4-methylpyridin-2-yl)-1-methyl-1,6-diazaspiro[3.3]heptane (Intermediate A-42)

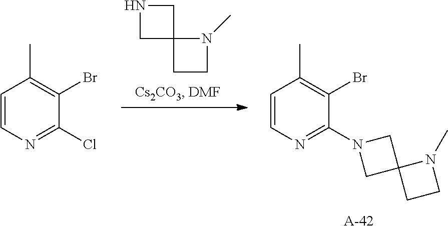

A mixture of 3-bromo-2-chloro-4-picoline (1.1 g, 5.33 mmol, Sigma-Aldrich. Inc.), 1-methyl-1,6-diazaspiro[3.3]heptane dihydrochloride (1.085 g, 5.86 mmol. AstaTech, Inc) and Cs₂CO₃ (6.94 g, 21.31 mmol) in DMF (10 mL) was heated at 98° C. for 18 h. The reaction was diluted with H₂O and extracted with EtOAc. The combined organics extracts were dried over Na₂SO₄, filtered, concentrated, and chromatographed on silica gel using 0-40% EtOAc in heptane to afford 6-(3-bromo-4-methylpyridin-2-yl)-1-methyl-1,6-diazaspiro[3.3]heptane. m/z (ESI): 282.00 (M+H)⁺.

The intermediates in the table below were prepared in a fashion similar to that described above.

| | | | m/z (ESI): |
|---|---|---|---|
| Int. # | Chemical Structure | Name | (M + H)⁺ |
| A-43 | 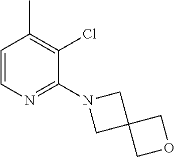 | 6-(3-chloro-4-methylpyridin-2-yl)-2-oxa-6- azaspiro[3.3]heptane | 225.1 |
| A-44 | 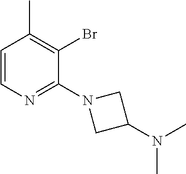 | 1-(3-bromo-4-methylpyridin-2-yl)-N,N- dimethylazetidin-3-amine | 270.0 |
| A-45 | 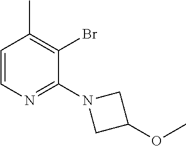 | 3-bromo-2-(3-methoxyazetidin-1-yl)-4- methylpyridine | 257.1 |
| A-46 | 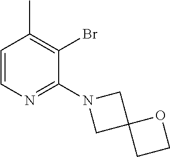 | 6-(3-bromo-4-methylpyridin-2-yl)-1-oxa-6- azaspiro[3.3]heptane | 269.0 |
| A-47 | 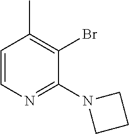 | 2-(azetidin-1-yl)-3-bromo-4-methylpyridine | 227.1 |

### 4-Chloro-3-cyclopropyl-5-methylpyridazine (Intermediate A-48)

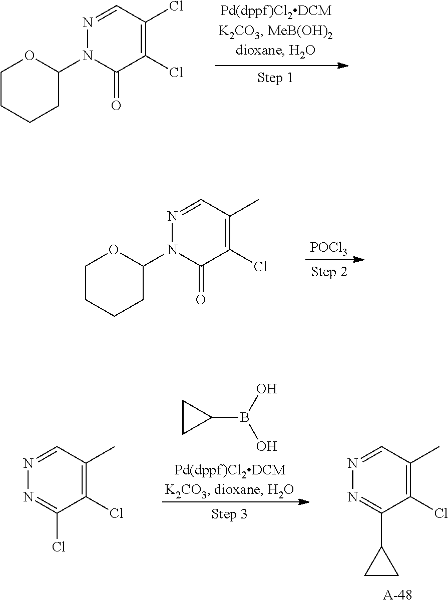

Step 1. A mixture of 4,5-dichloro-2-(tetrahydro-2h-pyran-2-yl)pyridazin-3(2H)-one (7.0 g, 28.1 mmol, Ambeed, Inc.), methylboronic acid (1.682 g, 28.1 mmol, Combi-Blocks, Inc.), Pd (dppf)Cl₂ DCM (1.377 g, 1.686 mmol, Combi-Blocks, Inc.) and potassium carbonate (9.71 g, 70.3 mmol) was purged with N₂ before the addition of 1,4-dioxane/H₂O (15/1.5 mL), and the resulting mixture was heated at 100° C. for 2 h. More boronic acid was added (0.15 eq) and the heating continued for 1 h more. The reaction was brought to rt, diluted with H₂O and extracted with EtOAc. The combined organic extracts were dried over Na₂SO₄, filtered, concentrated and chromatographed on silica gel using 0-70% EtOAc in heptane to afford 5-chloro-4-methyl-2-(tetrahydro-2H-pyran-2-yl)pyridazin-3(2H)one m/z (ESI): 251.05 (M+23)⁺ and 4-chloro-5-methyl-2-(tetrahydro-2H-pyran-2-yl)pyridazin-3(2H)-one. m/z (ESI): 251.05 (M+23)⁺. ¹H NMR (500 MHz, DMSO-d₆) δ ppm 7.97 (s, 1H), 5.87 (dd, J=10.6, 2.1 Hz, 1H), 3.86-4.01 (m, 1H), 3.53-3.69 (m, 1H), 2.28 (s, 3H), 2.03-2.11 (m, 1H), 1.91-1.98 (m, 1H), 1.63-1.73 (m, 2H), 1.48-1.54 (m, 2H).

Step 2. A mixture of 4-chloro-5-methyl-2-(tetrahydro-2H-pyran-2-3l)pyridazin-3(2H)-one (1.85 g, 8.09 mmol) and POCl₃ (24.81 g, 15.13 mL, 162 mmol, Sigma-Aldrich, Inc.) was heated to reflux for 1 h. The mixture was brought to rt, slowly poured into cold water and basified with solid K₂CO₃ to pH>8 The resulting mixture was extracted with -EtOAc. The combined organics were dried over Na₂SO₄, filtered, concentrated and chromatographed on silica gel using 0-80% EtOAc in heptane to afford 3,4-dichloro-5-methylpyridazine. m/z (ESI): 163.15 (M+H)⁺.

Step 3. A mixture of 3,4-dichloro-5-methylpyridazine (0.480 g, 2.94 mmol), cyclopropylboronic acid (0.278 g, 3.24 mmol, Combi-Blocks, Inc.), Pd(dppf)Cl₂ (0.144 g, 0.177 mmol, Combi-Blocks. Inc.) and potassium carbonate (1.017 g, 7.36 mmol, Sigma-Aldrich, Inc.) was purged with N₂ before the addition of 1,4-dioxane/H₂O (10/1.0 mL), and the resulting mixture was heated at 95° C. for 1 h. More boronic acid was added as needed to bring the reaction to completion. The reaction was brought to it, diluted with H₂O, and extracted with EtOAc. The combined organics were dried over Na₂SO₄, filtered. concentrated and chromatographed on silica gel using 0-80% EtOAc in heptane to afford 4-chloro-3-cyclopropyl-5-methylpyridazine. m/z (ESI): 169.15 (M+H)⁺.

The intermediate in the table below was prepared in a fashion similar to that described above.

| | | | m/z (ESI): |
|---|---|---|---|
| Int. # | Chemical Structure | Name | (M + H)⁺ |
| A-49 | 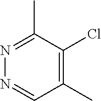 | 4-chloro-3,5-dimethylpyridazine | 143.0 |

### 3-Chloro-4-methyl-2-(oxetan-3-yl)pyridine (Intermediate A-50)

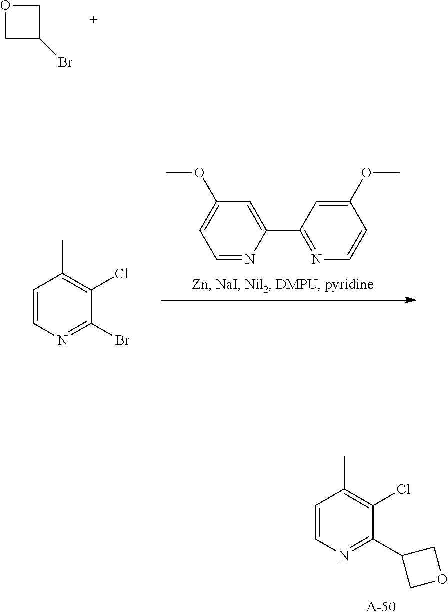

Nickel(II) iodide hydrate (0.469 g, 1.114 mmol, Combi-Blocks. Inc), 4,4-dimethoxy-2,2-bipyridine (0.241 g, 1.114 mmol, Sigma-Aldrich. Inc.). sodium iodide (0.417 g, 2.78 mmol, Sigma-Aldrich. Inc.), 2-bromo-3-chloro-4-methylpyridine (2.30 g, 11.14 mmol, Combi-Blocks, Inc.) and zinc dust (1.457 g, 22.28 mmol, Sigma-Aldrich, Inc.) were added to a flask followed by the addition of pyridine (0.088 g, 0.090 mL, 1.114 mmol, Sigma-Aldrich, Inc) and DMPU (20 ml). After stirring at rt for 5 mm, the mixture was heated at 60° C. for 18 h. The reaction mixture was diluted with EtOAc and filtered through celite. The filtrate was washed with H₂O and the organic phase was separated and dried over Na₂SO₄, filtered, concentrated and chromatographed on silica gel using 0-30% EtOAc in heptane to afford 3-chloro-4-methyl-2-(oxetan-3-yl)pyridine. m/z (ESI): 184.15 (M+H)⁺.

### 6-(3-Bromo-5-fluoro-4-methylpyridin-2-yl)-2-oxa-6-azaspiro[3.3]heptane (Intermediate A-51)

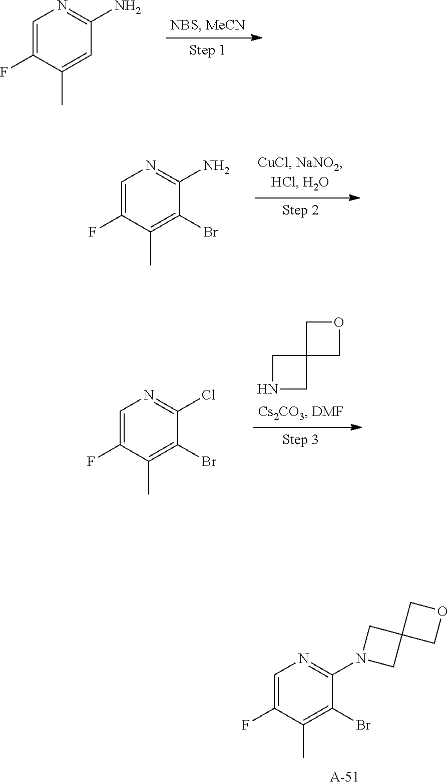

Step 1. To a suspension of 2-amino-5-fluoro-4-picoline (1.02 g, 8.09 mmol, AK Scientific, Inc.) in MeCN (10 mL) was added NBS (1.439 g, 8.09 mmol, Sigma-Aldrich, Inc.) and the resulting mixture was stirred at rt for 2 h. The reaction was concentrated and chromatographed on silica gel using 0-20% (3:1 EtOAc/EtOH) in heptane to afford 3-bromo-5-fluoro-4-methylpyridin-2-amine. m/z (ESI): 205.00 (M+H)⁺. ¹H NMR (400 MHz, DMSO-4) a ppm 7.90 (s, 1H), 6.04 (br s, 2H), 2.25 (d, J=2.1 Hz, 3H).

Step 2. To a mixture of conc. HCU/H₂O (6.6 mL) was added 3-bromo-5-fluoro-4-methylpyridin-2-amine (0.801 g, 3.91 mmol) followed by the addition of sodium nitrite (0.809 g, 11.72 mmol. Sigma-Aldrich, Inc.) and copper chloride (1.547 g, 15.63 mmol. Strem Chemicals, Inc.) and the resulting mixture was stirred at rt for 18 h. The reaction was brought to 0° C., diluted with EtOAc and basified with 1N NaOH. The mixture was filtered to remove the solids and the filtrate was extracted with EtOAc and the combined organic were dried over Na₂SO₄, filtered and concentrated to afford 3-bromo-2-chloro-5-fluoro-4-methylpyridine to be used as is. m/z (ESI): 223.90 (M+H)⁺. ¹H NMR (500 MHz, DMSO-d₆) δ ppm 8.45 (s, 1H), 2.40 (d, J=2.2 Hz, 3H).

Step 3. A mixture of 3-bromo-2-chloro-5-fluoro-4-methylpyridine (0.313 g, 1.394 mmol), 2-oxa-6-azaspiro[3.3]heptane (0.166 g, 1.673 mmol, Advanced ChemBlocks Inc.) and Cs₂CO₃ (1.363 g, 4.18 mmol, Combi-Blocks. Inc.) in DMF (10 mL) was heated at 98° C. for 5 h. The reaction was diluted with H₂O and extracted with EtOAc. The combined organics were dried over Na₂SO₄, filtered, concentrated and chromatographed on silica gel using 0-30% EtOAc in heptane to afford 6-(3-bromo-5-fluoro-4-methylpyridin-2-yl)-2-oxa-6-azaspiro[3.3]heptane. m/z (ESI): 287.0 (M+H)⁺.

### 3-Bromo-2-(2,2-difluorocyclopropyl)-4-methylpyridine (Intermediate A-52)

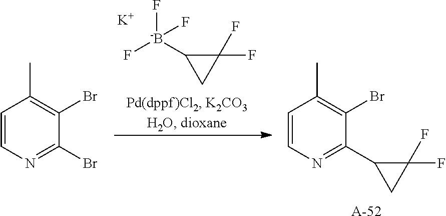

A mixture of 2,3-dibromo-4-methylpyridine (1.0 g, 3.99 mmol, Ambeed, Inc.), potassium (2,2-difluorocyclopropyl)-trifluoro-boranuide (0.770 g, 4.18 mmol, PharmaBlock, Inc.), potassium carbonate (1.377 g, 9.96 mmol, Sigma-Aldrich, Inc.) and Pd(dppf)Cl₂·DCM (0.325 g, 0.399 mmol, Oakwood Products, Inc.) was purged with N₂ before the addition of 1,4-dioxane/H₂O (10/1.0 mL), and the resulting mixture was heated at 95° C., over the weekend. The reaction was brought to rt, diluted with H₂O and extracted with EtOAc. The combined organics were dried over Na₂SO₄, filtered, concentrated and chromatographed on silica gel using 0-80/o EtOAc in heptane to afford 3-bromo-2-(2,2-difluorocyclopropyl)-4-methylpyridine. m/z (ESI): 247.90 (M+H)⁺.

### Tert-Butyl (1R,2S,5S)-2-methyl-3,8-diazabicyclo[3.2.1]octane-8-carboxylate (Intermediate A-53)

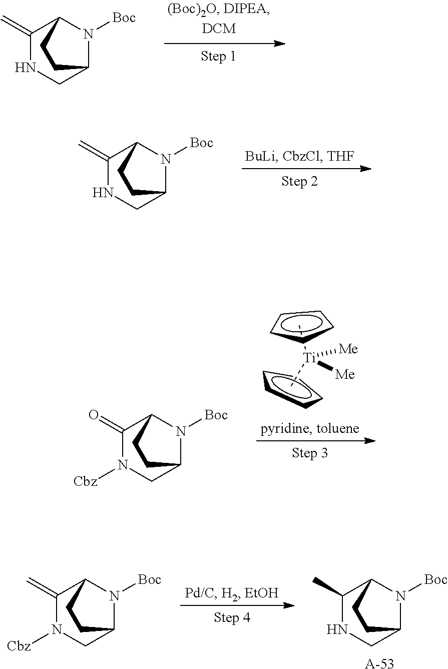

Step 1. To a vessel was added Boc-anhydride (2.447 mL, 10.54 mmol, Sigma-Aldrich. Inc.), 3,8-diazabicyclo[3.2.1]octan-2-one (1 g, 7.93 mmol), DIPEA (3.26 g, 4.4 mL, 25.2 mmol. Acros), and DCM (40 mL). The reaction mixture was stirred at it over 48 h. The reaction mixture was poured into sat. aq. NaHCO₃ and extracted with EtOAc and then MeTHF. The combined organic extracts were washed with sat. aq. NaCl, dried over MgSO₄, filtered, and concentrated in vacuo to give the crude material. The crude material was dissolved in DCM/MeOH and purified by chromatography, eluting with a gradient of 5% to 100% EtOAc in heptane then 0% to 20% MeOH in EtOAc to give tert-butyl 2-oxo-3,8-diazabicyclo[3.2.1]octane-8-carboxylate. ¹H NMR (400 MHz, CHLOROFORM-d) δ ppm 5.58 (br s, 1H) 4.37-4.59 (m, 2H) 3.74 (br d, J=9.30 Hz, 1H), 3.04 (dd, J=11.09, 2.15 Hz, 1H) 2.18-2.28 (m, 1H) 2.09-2.17 (m, 2H) 1.75-1.83 (m, 1H) 1.47 (s, 9H).

Step 2. To a vessel was added n-BuLi (1.6 M in hexanes) (2.91 mL, 4.37 mmol, Sigma-Aldrich. Inc.) and tert-butyl 2-oxo-3,8-diazabicyclo[3.2.1]octane-8-carboxylate (760 mg, 3.36 mmol) in THF (30 mL) at −78° C. The reaction mixture was stirred at −78° C. for 30 min before the addition of Cbz-Cl (1146 mg, 0.959 mL, 6.72 mmol, FluoroChem). The reaction mixture was allowed to slowly reach rt and stirred for 18 h. The reaction was quenched by addition of sat. aq. NH₄Cl and poured into sat. aq. NaHCO₃ and extracted with EtOAc. The combined organic phases were washed with sat. aq. NaCl, dried over MgSO₄, filtered and concentrated in vacuo to give the crude material. The crude material was purified by chromatography on silica gel, eluting with a gradient of 5% to 100% EtOAc in heptane to give 3-benzyl 8-(tert-butyl) 2-oxo-3,8-diazabicyclo[3.2.1]octane-3,8-dicarboxylate. m/z (ESI): 361.2 (M+H)⁺. ¹H NMR (400 MHz, CHLOROFORM-d) δ ppm 7.28-7.45 (m, 8H) 5.88-6.14 (m, 1H) 5.01-5.34 (m, 3H) 3.82-4.69 (m, 3H) 3.29-3.65 (m, 1H) 2.06-2.25 (m, 2H) 1.88-2.04 (m, 1H) 1.69-1.88 (m, 1H) 1.45 (s, 6H) 1.33 (s, 3H)

Step 3. To a vessel was added dimethylbis(cyclopentadienyl)titanium (8670 mg, 2.083 mmol. ABCR) and 3-benzyl 8-(tertbutyl) 2-oxo-3,8-diazabicyclo[3.2.1]octane-3,8-dicarboxylate (468 mg, 1.299 mmol) and pyridine (21 μL, 0.260 mmol) in toluene (13 mL). The reaction mixture was heated to 110° C. for 2 h. The reaction mixture was concentrated in vacuo. The crude material purified by chromatography, eluting with a gradient of 5% to 100% EtOAc in heptane to give 3-benzyl 8-(tert-butyl) 2-methylene-3,8-diazabicyclo[3.2.1]octane-3,8-dicarboxylate. m/z (ESI): 359.2 (M+H)⁺. ¹H NMR (400 MHz, CHLOROFORM-d) δ ppm 7.28-7.41 (m, 5H) 5.13-5.25 (m, 2H) 5.05 (br s, 1H) 4.85 (br s, 1H) 4.40 (br s, 1H) 4.26 (br s, 1H) 3.88 (dd, J=12.34, 1.01 Hz, 1H) 3.38 (br s, 1H) 1.93-2.07 (m, 2H) 1.69-1.85 (m, 2H) 1.46 (s, 9H)

Step 4. To a vessel was added EtOH (3 mL) and 3-benzyl 8-(tert-butyl) 2-methylene-3,8-diazabicyclo[3.2.1]octane-3,8-dicarboxylate (113 mg, 0.315 mmol) and Pd on carbon (67.1 mg, 0.063 mmol, Sigma-Aldrich, Inc.). The reaction mixture was sparged with H₂ for 5 min and stirred at rt for 18 h. The reaction mixture was filtered through a pad of celite, eluting with EtOAc, and concentrated in vacuo to give tert-butyl (1R,2S,5S)-2-methyl-3,8-diazabicyclo[3.2.1]octane-8-carboxylate, which was used without further purification.

### Piperidine Intermediates

### Tert-Butyl 4-(1-(2-methoxyethyl)-4-methyl-1H-pyrazol-5-yl)piperidine-1-carboxylate (Intermediate B-1)

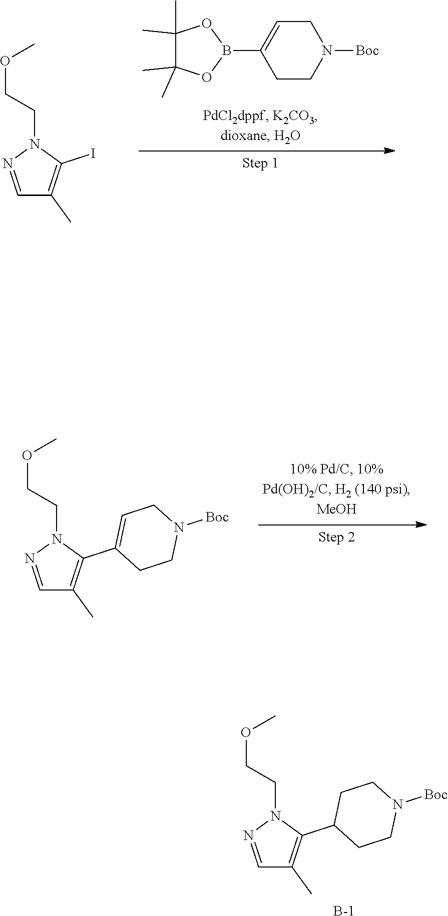

Step 1. 5-Iodo-1-(2-methoxyethyl)-4-methyl-1H-pyrazole (61 g, 229 mmol), tert-butyl 4-(4,4,5,5-tetramethyl-1,3,2-dioxaborolan-2-yl)-3,6-dihydropyridine-1(2H)-carboxylate (85 g, 275 mmol, Combi-Blocks, Inc.), potassium carbonate (95 g, 688 mmol), and PdCl₂(dppf)-DCM adduct (9.36 g, 11.46 mmol) were added to a flask with degassed dioxane (976 mL) and H₂O (244 mL). This mixture was heated at 80° C. for 16 h. The reaction mixture was quenched with ice cold water (2000 mL) and extracted with EtOAc (2×1000 mL). The combined organic extracts were dried over Na₂SO₄, filtered, and concentrated under reduced pressure. The crude product was purified by chromatography (silica, 30 to 50% EtOAc:hexanes) to give tert-butyl 4-(1-(2-methoxyethyl)-4-methyl-1H-pyrazol-5-yl)-3,6-dihydropyridine-1(2H)-carboxylate. m/z (ESI): 322.1 (M+H)⁺. ¹H NMR (400 MHz, DMSO-d₆) δ 7.23 (s, 1H), 5.78 (s, 1H), 4.07 (t, 2H, J=5.5 Hz), 3.62 (t, 2H, J=5.5 Hz), 3.53 (t, 2H, J=5.6 Hz), 3.16 (s, 3H), 2.24 (td, 2H, J=5.7, 3.0 Hz), 1.99 (s, 2H), 1.8-1.9 (m, 3H), 1.44 (s, 9H).

Step 2. To a solution of tert-butyl 4-(1-(2-methoxyethyl)-4-methyl-1H-pyrazol-5-yl)-3,6-dihydropyridine-1(2H)-carboxylate (103 g, 320 mmol) in MeOH (2060 mL) was added palladium on carbon (30.7 g, 30 wt %, Hindustan Platinum) and palladium hydroxide on carbon (31.5 g, 30 wt %. Hindustan Platinum). The reaction mixture was stirred under hydrogen pressure (140 psi) at 50° C. for 72 h. The reaction mixture was cooled, filtered through a bed of celite, and washed with MeOH (6000 mL). The filtrate was concentrated under reduced pressure to give tert-butyl 4-(1-(2-methoxyethyl)-4-methyl-1H-pyrazol-5-yl)piperidine-1-carboxylate. m/z (ESI): 324.2 (M+H)⁺. ¹H NMR (400 MHz, DMSO-d₆) δ ppm 7.12 (s, 1H), 4.20 (t, 2H, J=5.4 Hz), 3.9-4.2 (m, 2H), 3.59 (t, 2H, J=5.4 Hz), 3.20 (s, 3H), 3.00 (ddt, 1H, J=16.0, 10.9, 4.9 Hz), 2.79 (s, 2H), 2.00 (s, 3H), 1.6-1.8 (m, 4H), 1.42 (s, 9H).

The intermediates in the table below were prepared in a fashion similar to that described above for intermediate B-1. Synthesis of the building blocks is either described above or from commercial sources.

| | | | m/z (ESI): |
|---|---|---|---|
| Int. # | Chemical Structure | Name | (M + H)⁺ |
| B-2 | 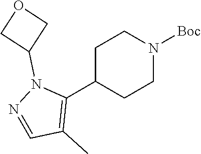 [SMILES: Cc1cnn(C2COC2)c1C1CCN(C(=O)OC(C)(C)C)CC1] | tert-butyl 4-(4-methyl-1-(oxetan-3-yl)-1H- pyrazol-5-yl)piperidine-1-carboxylate | 322.2 |
| B-3 | 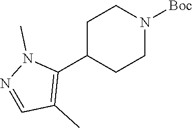 [SMILES: Cc1cnn(C)c1C1CCN(C(=O)OC(C)(C)C)CC1] | tert-butyl 4-(1,4-dimethyl-1H-pyrazol-5- yl)piperidine-1-carboxylate | 280.2 |
| B-4 | 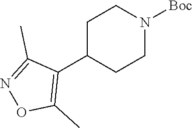 [SMILES: Cc1noc(C)c1C1CCN(C(=O)OC(C)(C)C)CC1] | tert-butyl 4-(3,5-dimethylisoxazol-4- yl)piperidine-1-carboxylate | 281.1 |
| B-5 | 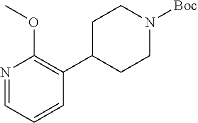 [SMILES: COc1ncccc1C1CCN(C(=O)OC(C)(C)C)CC1] | tert-butyl 4-(2-methoxypyridin-3-yl)piperidine- 1-carboxylate | 293.2 |
| B-6 | 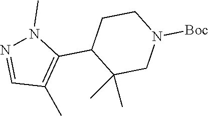 [SMILES: Cc1cnn(C)c1C1CCN(C(=O)OC(C)(C)C)CC1(C)C] | tert-butyl 4-(1,4-dimethyl-1H-pyrazol-5-yl)- 3,3-dimethylpiperidine-1-carboxylate | 308.1 |
| B-7 | 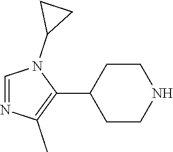 | 4-(1-cyclopropyl-4-methyl-1H-imidazol-5- yl)piperidine | 206.2 |
| B-8 | 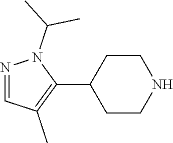 | 4-(1-isopropyl-4-methyl-1H-pyrazol-5- yl)piperidine | 208.1 |
| B-9 | 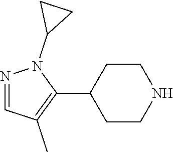 | 4-(1-cyclopropyl-4-methyl-1H-pyrazol-5- yl)piperidine | 206.2 |
| B-10 | 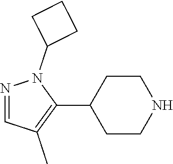 [SMILES: Cc1cnn(C2CCC2)c1C1CCNCC1] | 4-(1-cyclobutyl-4-methyl-1H-pyrazol-5- yl)piperidine | 220.1 |
| B-11 | 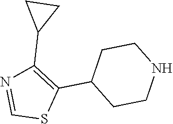 [SMILES: c1nc(C2CC2)c(C2CCNCC2)s1] | 4-cyclopropyl-5-(piperidin-4-yl)thiazole | 209.1 |
| B-12 | 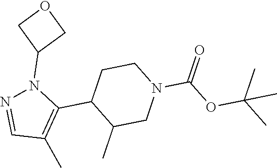 [SMILES: Cc1cnn(C2COC2)c1C1CCN(C(=O)OC(C)(C)C)CC1C] | tert-butyl 3-methyl-4-(4-methyl-1-(oxetan-3- yl)-1H-pyrazol-5-yl)piperidine-1-carboxylate | 336.1 |
| B-13 | 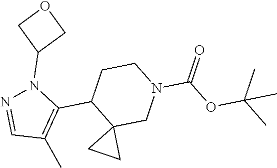 [SMILES: Cc1cnn(C2COC2)c1C1CCN(C(=O)OC(C)(C)C)CC12CC2] | tert-butyl 8-(4-methyl-1-(oxetan-3-yl)-1H- pyrazol-5-yl)-5-azaspiro[2.5]octane-5- carboxylate | 348.1 |
| B-14 | 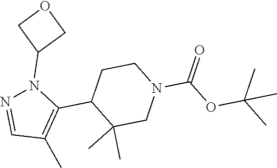 [SMILES: Cc1cnn(C2COC2)c1C1CCN(C(=O)OC(C)(C)C)CC1(C)C] | tert-butyl 3,3-dimethyl-4-(4-methyl-1-(oxetan- 3-yl)-1H-pyrazol-5-yl)piperidine-1-carboxylate | 350.1 |
| B-15 | 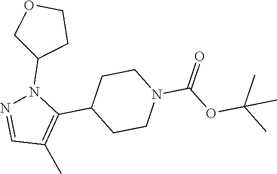 [SMILES: Cc1cnn(C2CCOC2)c1C1CCN(C(=O)OC(C)(C)C)CC1] | tert-butyl 4-(4-methyl-1-(tetrahydrofuran-3-yl)- 1H-pyrazol-5-yl)piperidine-1-carboxylate | 336.1 |
| B-16 | 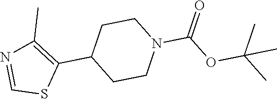 [SMILES: Cc1ncsc1C1CCN(C(=O)OC(C)(C)C)CC1] | tert-butyl 4-(4-methylthiazol-5-yl)piperidine-1- carboxylate | 283.2 |
| B-17 | 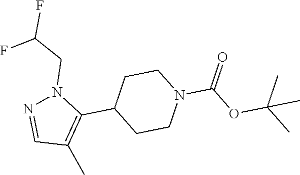 [SMILES: Cc1cnn(CC(F)F)c1C1CCN(C(=O)OC(C)(C)C)CC1] | tert-butyl 4-(1-(2,2-difluoroethyl)-4-methyl- 1H-pyrazol-5-yl)piperidine-1-carboxylate | 330.1 |
| B-18 | 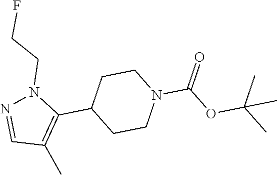 [SMILES: Cc1cnn(CCF)c1C1CCN(C(=O)OC(C)(C)C)CC1] | tert-butyl 4-(1-(2-fluoroethyl)-4-methyl-1H- pyrazol-5-yl)piperidine-1-carboxylate | 312.1 |
| B-19 | 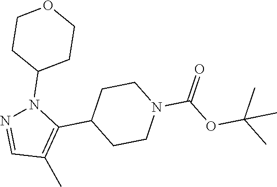 [SMILES: Cc1cnn(C2CCOCC2)c1C1CCN(C(=O)OC(C)(C)C)CC1] | tert-butyl 4-(4-methyl-1-(tetrahydro-2H-pyran- 4-yl)-1H-pyrazol-5-yl)piperidine-1-carboxylate | 350.0 |
| B-20 | 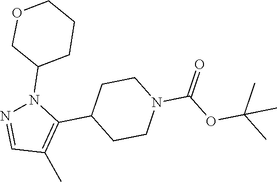 [SMILES: Cc1cnn(C2CCCOC2)c1C1CCN(C(=O)OC(C)(C)C)CC1] | tert-butyl 4-(4-methyl-1-(tetrabydro-2H-pyran- 3-yl)-1H-pyrazol-5-yl)piperidine-1-carboxylate | 350.2 |
| B-21 | 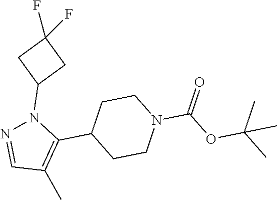 [SMILES: Cc1cnn(C2CC(F)(F)C2)c1C1CCN(C(=O)OC(C)(C)C)CC1] | tert-butyl 4-(1-(3,3-difluorocyclobutyl)-4- methyl-1H-pyrazol-5-yl)piperidine-1- carboxylate | 356.3 |
| B-22 | 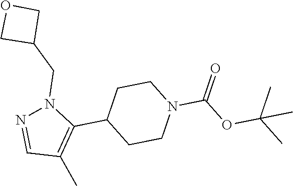 [SMILES: Cc1cnn(CC2COC2)c1C1CCN(C(=O)OC(C)(C)C)CC1] | tert-butyl 4-(4-methyl-1-(oxetan-3-ylmethyl)- 1H-pyrazol-5-yl)piperidine-1-carboxylate | 336.5 |
| B-23 | 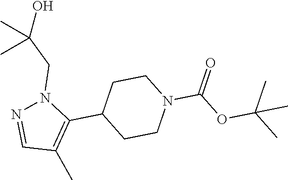 [SMILES: Cc1cnn(CC(C)(C)O)c1C1CCN(C(=O)OC(C)(C)C)CC1] | tert-butyl 4-(1-(2-hydroxy-2-methylpropyl)-4- methyl-1H-pyrazol-5-yl)piperidine-1- carboxylate | 338.0 |
| B-24 | 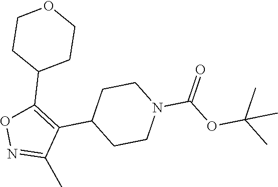 [SMILES: Cc1noc(C2CCOCC2)c1C1CCN(C(=O)OC(C)(C)C)CC1] | tert-butyl 4-(3-methyl-5-(tetrahydro-2H-pyran- 4-yl)isoxazol-4-yl)piperidine-1-carboxylate | 351.1 |
| B-25 | 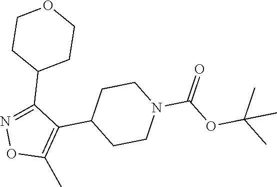 [SMILES: Cc1onc(C2CCOCC2)c1C1CCN(C(=O)OC(C)(C)C)CC1] | tert-butyl 4-(5-methyl-3-(tetrahydro-2H-pyran- 4-yl)isoxazol-4-yl)piperidine-1-carboxylate | 351.1 |
| B-26 | 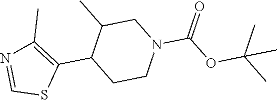 [SMILES: Cc1ncsc1C1CCN(C(=O)OC(C)(C)C)CC1C] | tert-butyl 3-methyl-4-(4-methylthiazol-5- yl)piperidine-1-carboxylate | 297.1 |
| B-27 | 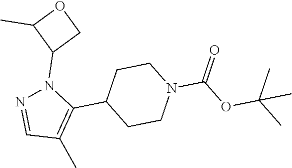 [SMILES: Cc1cnn(C2COC2C)c1C1CCN(C(=O)OC(C)(C)C)CC1] | tert-butyl 4-(4-methyl-1-(2-methyloxetan-3-yl)- 1H-pyrazol-5-yl)piperidine-1-carboxylate | 336.0 |
| B-28 | 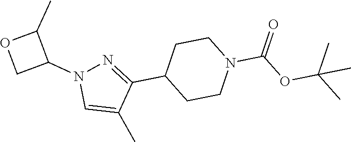 [SMILES: Cc1cn(C2COC2C)nc1C1CCN(C(=O)OC(C)(C)C)CC1] | tert-butyl 4-(4-methyl-1-(2-methyloxetan-3-yl)- 1H-pyrazol-3-yl)piperidine-1-carboxylate | 336.0 |
| B-29 | 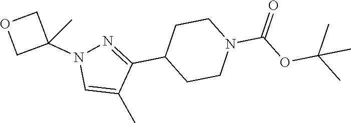 [SMILES: Cc1cn(C2(C)COC2)nc1C1CCN(C(=O)OC(C)(C)C)CC1] | tert-butyl 4-(4-methyl-1-(3-methyloxetan-3-yl)- 1H-pyrazol-3-yl)piperidine-1-carboxylate | 336.0 |
| B-30 | 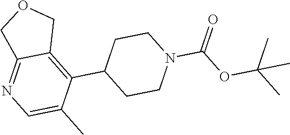 [SMILES: Cc1cnc2c(c1C1CCN(C(=O)OC(C)(C)C)CC1)COC2] | tert-butyl 4-(3-methyl-5,7-dihydrofuro[3,4- b]pyridin-4-yl)piperidine-1-carboxylate | 319.1 |
| B-31 | 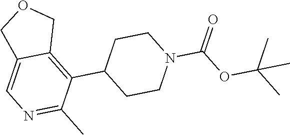 [SMILES: Cc1ncc2c(c1C1CCN(C(=O)OC(C)(C)C)CC1)COC2] | tert-butyl 4-(6-methyl-1,3-dihydrofuro[3,4- c]pyridin-7-yl)piperidine-1-carboxylate | 319.1 |
| B-32 | 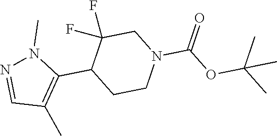 [SMILES: Cc1cnn(C)c1C1CCN(C(=O)OC(C)(C)C)CC1(F)F] | tert-butyl 4-(1,4-dimethyl-1H-pyrazol-5-yl)- 3,3-difluoropiperidine-1-carboxylate | 316.1 |
| B-33 | 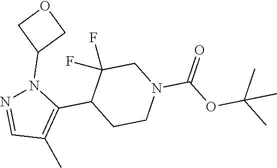 [SMILES: Cc1cnn(C2COC2)c1C1CCN(C(=O)OC(C)(C)C)CC1(F)F] | tert-butyl 3,3-difluoro-4-(4-methyl-1-(oxetan- 3-yl)-1H-pyrazol-5-yl)piperidine-1-carboxylate | 358.1 |
| B-34 | 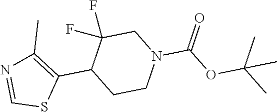 [SMILES: Cc1ncsc1C1CCN(C(=O)OC(C)(C)C)CC1(F)F] | tert-butyl 3,3-difluoro-4-(4-methylthiazol-5- yl)piperidine-1-carboxylate | 319.1 |
| B-53 | 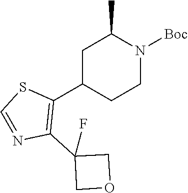 [SMILES: C[C@@H]1CC(c2scnc2C2(F)COC2)CCN1C(=O)OC(C)(C)C] | tert-butyl (2R)-4-(4-(3-fluorooxetan-3- yl)thiazol-5-yl)-2-methylpiperidine-1- carboxylate | 357.2 |
| B-54 | 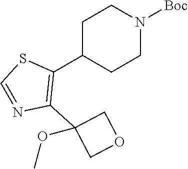 [SMILES: COC1(c2ncsc2C2CCN(C(=O)OC(C)(C)C)CC2)COC1] | tert-butyl 4-(4-(3-methoxyoxetan-3-yl)thiazol- 5-yl)piperidine-1-carboxylate | 355.3 |

### SFC Conditions for Chiral Separation

| | | | Racemic SM \| separation |
|---|---|---|---|
| Int.# | Chemical Structure | Name | condition |
| B-6-1 | 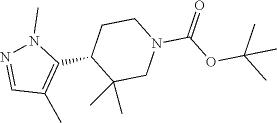 [SMILES: Cc1cnn(C)c1[C@@H]1CCN(C(=O)OC(C)(C)C)CC1(C)C] | tert-butyl (R)-4-(1,4-dimethyl- 1H-pyrazol-5-yl)-3,3- dimethylpiperidine-1- carboxylate | B-6 \| SFC (Chiralpak IG, 50 x 250 mm 5 μm, column with a mobile phase of 15% MeOH in liquid CO₂ using a flow rate of 200 mL/min) |
| | 1^{st}-eluting isomer | | |
| B-6-2 | 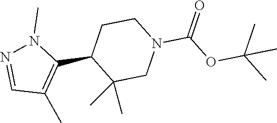 [SMILES: Cc1cnn(C)c1[C@H]1CCN(C(=O)OC(C)(C)C)CC1(C)C] | tert-butyl (S)-4-(1,4-dimethyl- 1H-pyrazol-5-yl)-3,3- dimethylpiperidine-1- carboxylate | B-6 \| SFC (Chiralpak IG, 50 x 250 mm 5 μm, column with a mobile phase of 15% MeOH in liquid CO₂ using a flow rate of 200 mL/min) |
| | 2^{nd}-eluting isomer | | |
| B-13-1 | 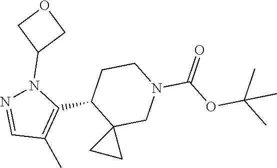 [SMILES: Cc1cnn(C2COC2)c1[C@@H]1CCN(C(=O)OC(C)(C)C)CC12CC2] | tert-butyl (R)-8-(4-methyl-1- (oxetan-3-yl)-1H-pyrazol-5-yl)- 5-azaspiro[2.5]octane-5- carboxylate | B-13 \| SFC (Chiralpak IG, 50 x 250 mm 5 μm, column with a mobile phase of 0.1% iPrNH₂ in MeOH using a flow rate of 120 mL/min) |
| | 1^{st}-eluting isomer | | |
| B-13-2 | 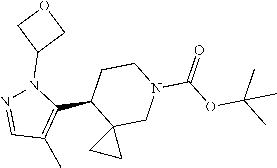 [SMILES: Cc1cnn(C2COC2)c1[C@H]1CCN(C(=O)OC(C)(C)C)CC12CC2] | tert-butyl (S)-8-(4-methyl-1- (oxetan-3-yl)-1H-pyrazol-5-yl)- 5-azaspiro[2.5]octane-5- carboxylate | B-13 \| SFC (Chiralpak IG, 50 x 250 mm 5 μm, column with a mobile phase of 0.1% iPrNH₂ in MeOH using a flow rate of 120 mL/min) |
| | 2^{nd}-eluting isomer | | |
| B-14-1 | 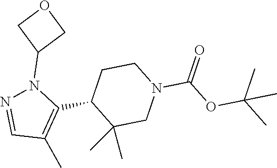 [SMILES: Cc1cnn(C2COC2)c1[C@@H]1CCN(C(=O)OC(C)(C)C)CC1(C)C] | tert-butyl (R)-3,3-dimethyl-4-(4- methyl-1-(oxetan-3-yl)-1H- pyrazol-5-yl)piperidine-1- carboxylate | B-14 \| SFC (Chiral Pak IC, 50 x 150 mm 5 μm, column with a mobile phase of 40% IPA in liquid CO₂ using a flow rate of 160 mL/min) |
| | 1^{st}-eluting isomer | | |
| B-14-2 | 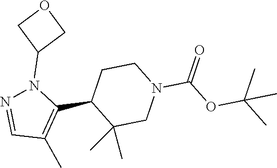 [SMILES: Cc1cnn(C2COC2)c1[C@H]1CCN(C(=O)OC(C)(C)C)CC1(C)C] | tert-butyl (S)-3,3-dimethyl-4-(4- methyl-1-(oxetan-3-yl)-1H- pyrazol-5-yl)piperidine-1- carboxylate | B-14 \| SFC (Chiral Pak IC, 50 x 150 mm 5 μm, column with a mobile phase of 40% IPA in liquid CO₂ using a flow rate of 160 ml/min) |
| | 2^{nd}-eluting isomer | | |
| B-26-1 | 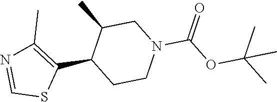 [SMILES: Cc1ncsc1[C@@H]1CCN(C(=O)OC(C)(C)C)C[C@@H]1C] | tert-butyl (3R,4R)-3-methyl-4- (4-methylthiazol-5- yl)piperidine-1-carboxylate | B-26 \| SFC (Chiralpak IG, 250 x 30 mm 5 μm, column with a mobile phase of 20% MeOH in liquid CO₂ using a flow rate of 100 mL/min) |
| | 1^{st}-eluting isomer | | |
| B-26-2 | 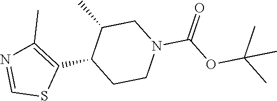 [SMILES: Cc1ncsc1[C@H]1CCN(C(=O)OC(C)(C)C)C[C@H]1C] | tert-butyl (3S,4S)-3-methyl-4- (4-methylthiazol-5- yl)piperidine-1-carboxylate | B-26 \| SFC (Chiralpak IG, 250 x 30 mm 5 μm, column with a mobile phase of 20% MeOH in liquid CO₂ using a flow rate of 100 mL/min) |
| | 2^{nd}-eluting isomer | | |
| B-27-1 | 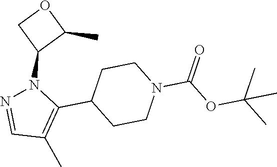 [SMILES: Cc1cnn([C@H]2CO[C@H]2C)c1C1CCN(C(=O)OC(C)(C)C)CC1] | tert-butyl 4-(4-methyl-1- ((2S,3S)-2-methyloxetan-3-yl)- 1H-pyrazol-5-yl)piperidine-1- carboxylate | B-27 \| SFC (Chiralpak IC, 150 x 50 mm 5 μm, column with a mobile phase of 20% MeOH in liquid CO₂ using a flow rate of 180 mL/min) |
| | 1^{st}-eluting isomer | | |
| B-27-2 | 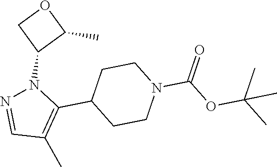 [SMILES: Cc1cnn([C@@H]2CO[C@@H]2C)c1C1CCN(C(=O)OC(C)(C)C)CC1] | tert-butyl 4-(4-methyl-1- ((2R,3R)-2-methyloxetan-3-yl)- 1H-pyrazol-5-yl)piperidine-1- carboxylate | B-27 \| SFC (Chiralpak IC, 150 × 50 mm 5 μm, column with a mobile phase of 20% MeOH in liquid CO₂ using a flow rate of 180 mL/min) |
| | 2^{nd}-eluting isomer | | |

### 2-(Oxetan-3-yl)-3-(piperidin-4-yl)pyridine (Intermediate B-35)

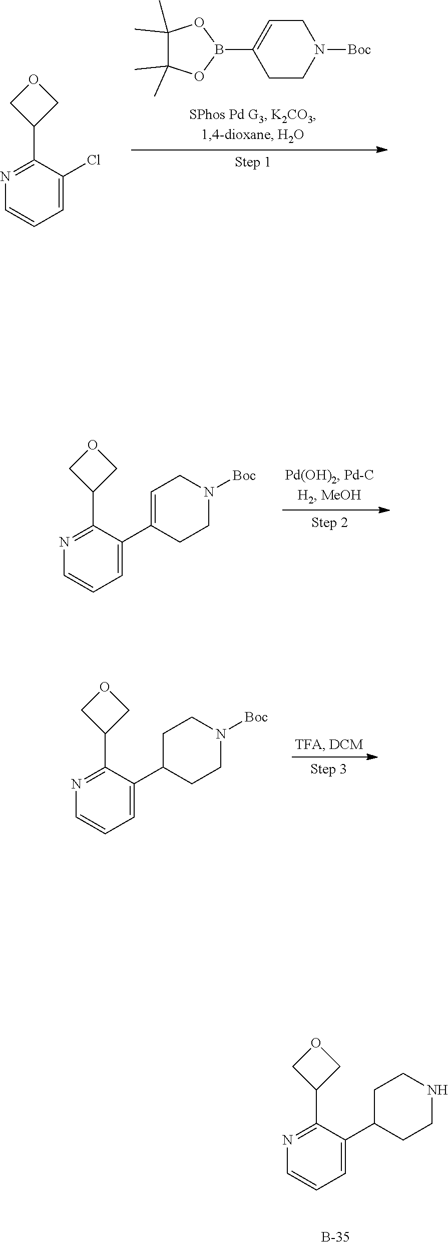

Step 1. Argon gas was purged through a stirred solution of 3-chloro-2-(oxetan-3-yl)pyridine (0.7 g, 4.13 mmol). tert-butyl 4-(4,4,5,5-tetramethyl-1,3,2-dioxaborolan-2-yl)-3,6-dihydropyridine-1(2H)-carboxylate (1.531 g, 4.95 mmol, Combi-Blocks. Inc.), and K₂CO₃ (1.711 g, 12.38 mmol Spectrochem) in 1,4-dioxane (10 mL) and H₂O (2 mL) for 15 min. SPhos Pd G3 (0.169 g, 0.206 mmol, Strem chemicals) was added to the reaction mixture and stirred at 100° C. for 16 h. The reaction mixture was quenched with H₂O (20 mL) and extracted with EtOAc (3<50 mL). The combined organic extracts were washed with brine, dried over Na₂SO₄, filtered, and concentrated under reduced pressure. The residue was purified on a Redi-Sep pre-packed silica gel column eluting with 30% to 40% EtOAc in pet ether to provide tert-butyl 2-(oxetan-3-yl)-3′,6′-dihydro-[3,4′-bipyridine]-1′(2′H)-carboxylate. m/z (ESI): 317.3 (M+H)⁺. ¹H NMR (400 MHz, Chloroform-d): δ ppm 8.61 (dd, 1H, J=4.8, 1.7 Hz), 7.42 (dd, 1H, J=7.7, 1.8 Hz), 7.19 (dd, 1H, J=7.7, 4.8 Hz), 5.52 (s, 1H), 5.02 (dd, 2H, J=7.2, 5.6 Hz), 4.93 (dd, 2H, J=8.4, 5.5 Hz), 4.5-4.6 (m, 1H), 4.05 (q, 2H, J=3.0 Hz), 3.64 (t, 2H, J=5.6 Hz), 2.30 (s, 21H), 1.52 (s, 9H).

Step 2. To a stirred solution of tert-butyl 2-(oxetan-3-yl)-3′,6′-dihydro-[3,4′-bipyridine]-1′(2′H)-carboxylate (0.75 g, 2.37 mmol) in MeOH (10 mL) was added 10% palladium on carbon (0.08 g, 10.7% w/w. Sigma-Aldrich, Inc.) and 20% Pd(OH)₂ on carbon (0.08 g, 10.7% w/w, Sigma-Aldrich, Inc.) under a nitrogen atmosphere. The reaction mixture was stirred for 12 h under 14 psi H₂ pressure. The reaction mixture was filtered through a celite pad and washed with MeOH (75 mL). The filtrate was concentrated under reduced pressure to provide crude tert-butyl 4-(2-(oxetan-3-yl)pyridin-3-yl)piperidine-1-carboxylate, which was used in the next step without purification. m/z (ESI): 319.2 (M+H)⁺.

Step 3. To a stirred solution of tert-butyl 4-(2-(oxetan-3-yl)pyridin-3-yl)piperidine-1-carboxylate (0.28 g, 0.879 mmol) in DCM (5 mL) was added TFA (0.6 mL, 7.79 mmol, Symax Laboratories) and the reaction mixture was stirred at rt for 3 h. The reaction mixture was concentrated and triturated with diethyl ether (10 mL) to obtain 2-(oxetan-3-yl)-3-(piperidin-4-yl)pyridine trifluoroacetate salt, which was used in next step without further purification. m/z (ESI): 219.3 (M+H)⁺.

The intermediate in the table below was prepared in a fashion similar to that described above for intermediate B-35. Synthesis of the building blocks is either described above or from commercial sources.

| | | | m/z (ESI): |
|---|---|---|---|
| Int. # | Chemical Structure | Name | (M + H)⁺ |
| B-36 | 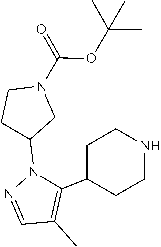 [SMILES: Cc1cnn(C2CCN(C(=O)OC(C)(C)C)C2)c1C1CCNCC1] | tert-butyl 3-(4-methyl-5-(piperidin-4-yl)-1H- pyrazol-1-yl)pyrrolidine-1-carboxylate | 335.3 |
| B-55 | 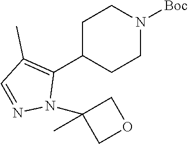 [SMILES: Cc1cnn(C2(C)COC2)c1C1CCN(C(=O)OC(C)(C)C)CC1] | tert-butyl 4-(4-methyl-1-(3-mctbyloxetan-3-yl)- 1H-pyrazol-5-yl)piperidine-1-carboxylate | 336.1 |
| B-56 | 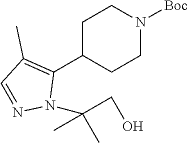 [SMILES: Cc1cnn(C(C)(C)CO)c1C1CCN(C(=O)OC(C)(C)C)CC1] | tert-butyl 4-(1-(1-hydroxy-2-methylpropan-2- yl)-4-methyl-1H-pyrazol-5-yl)piperidine-1- carboxylate | 338.1 |

### Tert-Butyl 4-(4-chloro-1-methyl-1H-pyrazol-5-yl)piperidine-1-carboxylate (Intermediate B-37)

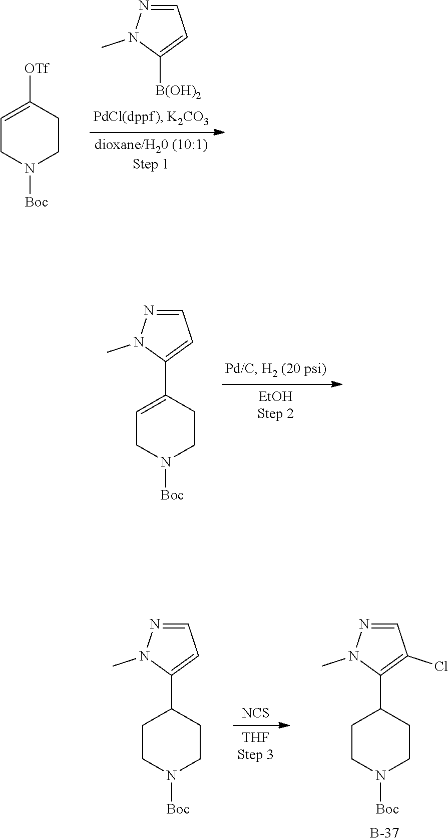

Step 1. To a vial was added potassium carbonate (417 mg, 3.02 mmol, Sigma-Aldrich, Inc.), Pd(dppf)Cl₂·DCM (123 mg, 0, 151 mmol, Combi-Blocks. Inc.). (1-methyl-1H-pyrazol-5-yl)boronic acid (380 mg, 3.02 mmol, PharmaBlock. Inc.), and 4-trifluoromethanesulfonyloxy-3,6-dihydro-2h-pyridine-1-carboxylic acid tert-butyl ester (500 mg, 1.509 mmol, J & W Pharmlab). The reaction vessel was evacuated and backfilled with N₂. To the reaction vessel was added 1,4-dioxane (5 mL) and H₂O (0.5 mL) and the reaction mixture was heated to 100° C., and stirred for 30 min. The reaction mixture was diluted with H₂O and extracted with EtOAc. The organic extracts were filtered through a plug of anhydrous Na₂SO₄ and concentrated in vacuo. The crude material was purified by chromatography through a Redi-Sep pre-packed silica gel column, eluting with a gradient of 0% to 50% EtOAc in heptane, to provide tert-butyl 4-(1-methyl-1H-pyrazol-5-yl)-3,6-dihydropyridine-1(2H)-carboxylate. m/z (ESI): 264.2 (M+H)⁺.

Step 2. To a hydrogenation vessel was added palladium on carbon (12 mg, 0.113 mmol, Sigma-Aldrich. Inc.) and the reaction vessel was purged with N₂ for 2 min. To the reaction vessel was added a solution of tert-butyl 4-(1-methyl-1H-pyrazol-5-yl)-3,6-dihydropyridine-1(2H)-carboxylate (297 mg, 1.128 mmol) in EtOH (4 mL) and the headspace was purged with H₂ 3 times. The reaction mixture was then placed under 20 psi of H₂ and stirred at rt for 18 h. The reaction mixture was filtered over celite, eluting with EtOAc, and the filtrate was concentrated in vacuo. The crude material was absorbed onto a plug of silica gel and purified by chromatography through a Redi-Sep pre-packed silica gel column, eluting with a gradient of 0% to 40% EtOAc in heptane, to provide tert-butyl 4-(1-methyl-1H-pyrazol-5-yl)piperdine-1-carboxylate. m/z (ESI): 266.2 (M+H)⁺.

Step 3. A solution of tert-butyl 4-(1-methyl-1H-pyrazol-5-yl)piperidine-1-carboxylate (500 mg, 1.884 mmol) and NCS (503 mg, 3.77 mmol, Sigma-Aldrich, Inc.) in THF (5 mL) was heated to 80° C. and stirred for 2 h. The reaction mixture was diluted with satd. Aq. NaHCO₃ and extracted with EtOAc. The organic extracts were filtered over a plug of Na₂SO₄ and concentrated in vacuo. The crude material was purified by chromatography through a Redi-Sep pre-packed silica gel column, eluting with a gradient of 0% to 10% EtOAc in heptane, to provide tert-butyl 4-(4-chloro-1-methyl-1H-pyrazol-5-yl)piperidine-1-carboxylate. m/z (ESI): 300.0 (M+H)⁺.

The intermediates in the table below were prepared in a fashion similar to that described above for intermediate B-37. Synthesis of the building blocks is either described above or from commercial sources.

| | | | m/z (ESI): |
|---|---|---|---|
| Int. # | Chemical Structure | Name | (M + H)⁺ |
| B-38 | 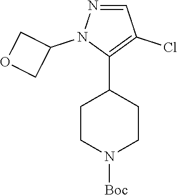 [SMILES: CC(C)(C)OC(=O)N1CCC(c2c(Cl)cnn2C2COC2)CC1] | tert-butyl 4-(4-chloro-1-(oxetan-3-yl)-1H-pyrazol- 5-yl)piperidine-1-carboxylate | 342.0 |
| B-39 | 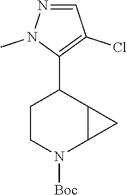 [SMILES: Cn1ncc(Cl)c1C1CCN(C(=O)OC(C)(C)C)C2CC12] | tert-butyl 5-(4-chloro-1-methyl-1H-pyrazol-5-yl)- 2-azabicyclo[4.1.0]heptane-2-carboxylate | 312.2 |
| B-57 | 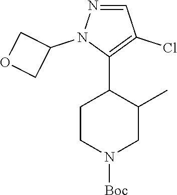 [SMILES: CC1CN(C(=O)OC(C)(C)C)CCC1c1c(Cl)cnn1C1COC1] | tert-butyl 4-(4-chloro-1-(oxetan-3-yl)-1H-pyrazol- 5-yl)-3-methylpiperidine-1-carboxylate | 256.0 |

### Tert-Butyl 5-(4-methyl-1-(oxetan-3-yl)-1H-pyrazol-5-yl)-2-azabicyclo[4.1.0]heptane-2-carboxylate (Intermediate B-40)

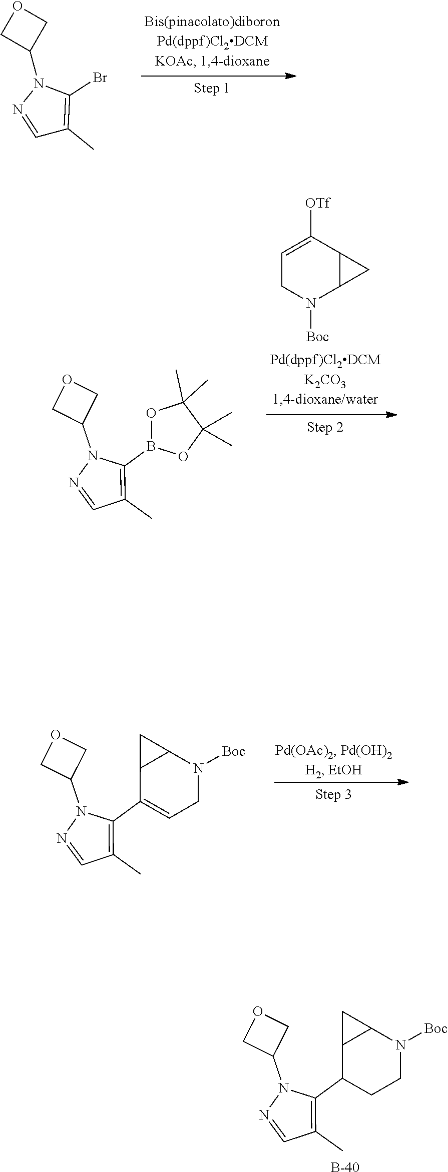

Step 1. A suspension of 5-bromo-4-methyl-1-(oxetan-3-yl)-1H-pyrazole (1 g, 4.61 mmol), bis(pinacolato)diboron (1.521 g, 5.99 mmol, Spectrochem) and potassium acetate (1.356 g, 13.82 mmol, Sigma-Aldrich, Inc.) in 1,4-dioxane (20 mL) was purged with argon gas for 15 min, Pd(dppf)Cl₂—CH₂Cl₂ (0.263 g, 0.322 mmol, Hindustan Platinum) was added to the suspension and the reaction mixture was stirred at 85° C. for 16 h. The reaction mixture was concentrated under reduced pressure. The crude residue was purified by chromatography (silica, 3% to 10% EtOAc in hexanes) to give 4-methyl-1-(oxetan-3-yl)-5-(4,4,5,5-tetramethyl-1,3,2-dioxaborolan-2-yl)-1H-pyrazole. m/z (ESI): 265.1 (M+H)⁺. ¹H NMR (400 MHz, Chloroform-d) δ ppm 7.46 (s, 1H), 5.99 (tt, 1H, J=7.6, 6.4 Hz), 5.19 (Id, 2H, J=6.6, 2.0 Hz), 4.90-5.10 (m, 2H), 2.24 (s, 3H), 1.35 (s, 12H).

Step 2. A suspension of tert-butyl (1S,6S)-5-(((trifluoromethyl)sulfonyl)oxy)-2-azabicyclo[4.1.0]hept-4-ene-2-carboxylate (1.354 g, 3.94 mmol), 4-methyl-1-(oxetan-3-yl)-5-(4,4,5,5-tetramethyl-1,3,2-dioxaborolan-2-yl)-1H-pyrazole (0.5 g, 1.893 mmol) and K₂CO₃ (0.654 g, 4.73 mmol, Chempure) in 1,4-dioxane (10 mL) and H₂O (2 mL) was purged with argon gas for 15 min, Pd(dppf)Cl₂—CH₂Cl₂ (0.064 g, 0.079 mmol, Hindustan Platinum) was added to the reaction mixture and the resulting mixture was stirred at 100° C. for 16 t. The reaction mixture was quenched with H₂O (10 mL) and extracted with EtOAc (2×20 mL). The organic extract was washed with brine, dried over anhydrous Na₂SO₄, filtered and concentrated under reduced pressure. The crude residue was purified by chromatography (silica, 0% to 30% EtOAc:hexanes) to give tert-butyl (1S,6S)-5-(4-methyl-1-(oxetan-3-yl)-1H-pyrazol-5-yl)-2-azabicyclo[4.1.0]hept-4-ene-2-carboxylate. m/z (ESI): 332.0 (M+H)⁺.

Step 3. To a solution of tert-butyl (1S,6S)-5-(4-methyl-1-(oxetan-3-yl)-1H-pyrazol-5-yl)-2-azabicyclo[4.1.0]hept-4-ene-2-carboxylate (0.65 g, 1.961 mmol) in EtOH (10 mL) was added 20% palladium hydroxide on carbon (0.055 g, 8.46% w/w, Hindustan Platinum) and palladium(II) acetate (0.022 g, 0.098 mmol, Combi-Block. Inc.) under a nitrogen atmosphere. The reaction mixture was stirred under hydrogen bladder pressure at 25° C. for 16 h. The reaction mixture was filtered through a plug of celite, washed with MeOH (15 mL), and concentrated under reduced pressure. The crude residue was purified by chromatography (silica, 10% to 20% EtOAc in hexanes) to give tert-butyl (1S,6S)-5-(4-methyl-1-(oxetan-3-yl)-1H-pyrazol-5-yl)-2-azabicyclo[4.1.0]heptane-2-carboxylate. m/z (ESI): 334.0 (M+H)⁺.

The intermediates in the table below were prepared in a fashion similar to that described above for intermediate B-40. Synthesis of the building blocks is either described above or from commercial sources.

| | | | m/z (ESI): |
|---|---|---|---|
| Int. # | Chemical Structure | Name | (M + H)⁺ |
| B-41 | 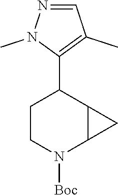 | tert-butyl 5-(1,4-dimethyl-1H-pyrazol-5-yl)-2- azabicyclo[4.1.0]heptane-2-carboxylate | 292.1 |
| B-42 | 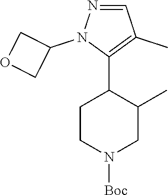 [SMILES: Cc1cnn(C2COC2)c1C1CCN(C(=O)OC(C)(C)C)CC1C] | tert-butyl 3-methyl-4-(4-methyl-1-(oxetan-3-yl)- 1H-pyrazol-5-yl)piperidine-1-carboxylate | 336.1 |
| B-43 | 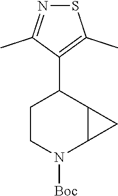 | tert-butyl 5-(3,5-dimethylisothiazol-4-yl)-2- azabicyclo[4.1.0]heptanc-2-carboxylate | 309.2 |
| B-44 | 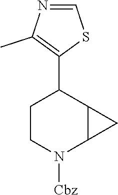 [SMILES: Cc1ncsc1C1CCN(C(=O)OCc2ccccc2)C2CC12] | benzyl 5-(4-methylthiazol-5-yl)-2- azabicyclo[4.1.0]heptane-2-carboxylate | 329.1 |
| B-58 | 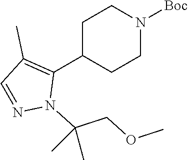 [SMILES: COCC(C)(C)n1ncc(C)c1C1CCN(C(=O)OC(C)(C)C)CC1] | tert-butyl 4-(1-(1-methoxy-2-methylpropan-2-yl)-4- methyl-1H-pyrazol-5-yl)piperidine-1-carboxylate | 352.3 |

### SFC Conditions for Chiral Separation

| | | | Racemic SM \| |
|---|---|---|---|
| Int.# | Chemical Structure | Name | separation condition |
| B-42-1 | 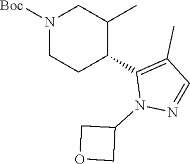 [SMILES: Cc1cnn(C2COC2)c1[C@@H]1CCN(C(=O)OC(C)(C)C)CC1C] | tert-butyl (3S,4R)-3-methyl-4-(4- methyl-1-(oxetan-3-yl)-1H- pyrazol-5-yl)piperidine-1- carboxylate | SFC (Daicel Chiralpak AD, 250 x 30 mm, 10 μm column with a mobile phase of 5-25% MeOH in liquid CO₂ using a flow rate of 70 mL/min) |
| | 1^{st}-eluting isomer | | |
| B-44-1 | 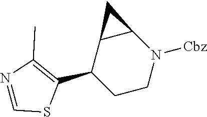 [SMILES: Cc1ncsc1[C@@H]1CCN(C(=O)OCc2ccccc2)[C@@H]2C[C@@H]21] | benzyl (1R,5R,6R)-5-(4- methylthiazol-5-yl)-2- azabicyclo[4.1.0]heptane-2- carboxylate | B-44 \| SFC (Chiralpak IG, 250 x 30 mm 5 μm, column with a mobile phase of 20% MeOH in liquid CO₂ using a flow rate of 100 mL/min) |
| | 1^{st}-eluting isomer | | |
| B-44-2 | 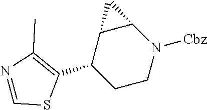 [SMILES: Cc1ncsc1[C@H]1CCN(C(=O)OCc2ccccc2)[C@H]2C[C@@H]12] | benzyl (1S,5S,6S)-5-(4- methylthiazol-5-yl)-2- azabicyclo[4.1.0]heptane-2- carboxylate | B-44 \| SFC (Chiralpak IG, 250 x 30 mm 5 μm, column with a mobile phase of 20% MeOH in liquid CO₂ using a flow rate of 100 mL/min) |
| | 2^{nd}-eluting isomer | | |

### Tert-Butyl 4-(2-methyl-1H-imidazol-1-yl)piperidine-1-carboxylate (Intermediate B-45)

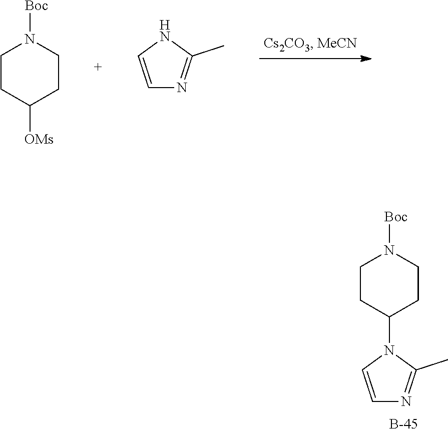

To a 4-mL reaction vial was added cesium carbonate (22.05 g, 6.30 mmol, Sigma-Aldrich, Inc.), tert-butyl 4-((methylsulfonyl)oxy)piperidine-1-carboxylate (220 mg, 0.788 mmol, Sigma-Aldrich, Inc.), and 2-methylimidazole (64.7 mg, 0.788 mmol, Combi-Blocks. Inc.) in MeCN (2 mL). The mixture was stirred at 85° C. for 2 h, then the crude material was filtered and concentrated. The crude product was used without further purification, m/z (ESI): 266.2 (M+H)⁺.

### Tert-Butyl 4-(5-methyl-3-(oxetan-3-yl)isothiazol-4-yl)piperidine-1-carboxylate (Intermediate B-46)

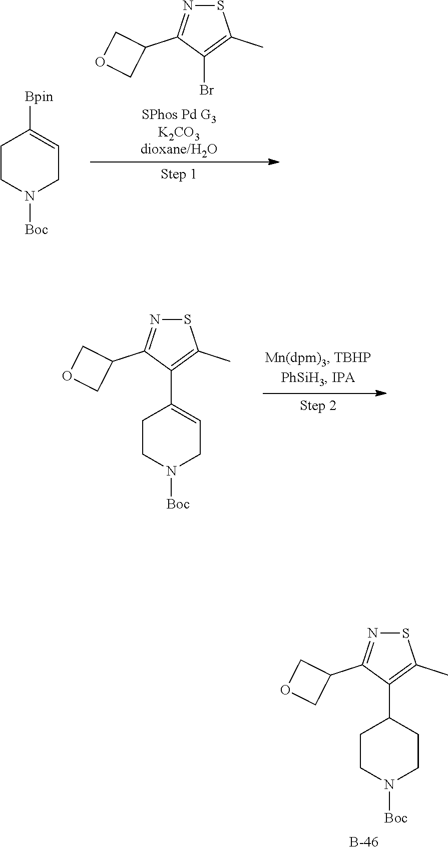

Step 1. To a vial was added potassium carbonate (0.328 g, 2.37 mmol, Sigma-Aldrich. Inc.). (1-tert-butoxycarbonyl-1,2,3,6-tetrahydropyridin-4-yl)boronic acid pinacol ester (0.367 g, 1.187 mmol, Combi-Blocks. Inc.), 4-bromo-3-methyl-5-(oxetan-3-yl)isothiazole (0.28 g, 1.187 mmol), and SPhos Pd G3 (0.103 g, 0.119 mmol, Sigma-Aldrich, Inc.). The reaction vessel was evacuated and backfilled with N₂. To the reaction vessel was added 1,4-dioxane (3 mL) and H₂O (0.3 mL) and the reaction mixture was heated to 100° C. for 5 h. The reaction mixture was diluted with H₂O and extracted with EtOAc. The organic extract was filtered through a plug of anhydrous Na₂SO₄ and concentrated in vacuo. The crude material was purified by chromatography through a Redi-Sep pre-packed silica gel column, eluting with a gradient of 0% to 40% EtOAc in heptane, to provide tert-butyl 4-(3-methyl-5-(oxetan-3-yl)isothiazol-4-yl)-3,6-dihydropyridine-1(2H)-carboxylate. m/z (ESI): 337.2 (M+H)⁺.

Step 2. To a 0° C. solution of tris(2,2,6,6-tetramethyl-3,5-heptanedionato)manganese(III) (0.029 g, 0.049 mmol, Sigma-Aldrich. Inc.) and tert-butyl 4-(3-methyl-5-(oxetan-3-yl)isothiazol-4-yl)-3,6-dihydropyridine-1(2H)-carboxylate (0.33 g, 0.974 mmol) in degassed IPA (5 mL, sparged with N₂ for 15 min) was added isopropoxy(phenyl)silane (0.262 mL, 1.461 mmol, Sigma-Aldrich. Inc) and t-butyl hydroperoxide (0.292 mL, 1.461 mmol, Sigma-Aldrich, Inc.) dropwise and the reaction mixture was stirred at rt for 18 h. The reaction mixture was quenched with NH₄OH and diluted with H₂O and brine. The reaction mixture was extracted with EtOAc and the organic extracts were washed with brine, filtered through a plug of Na₂SO₄, and concentrated in vacuo. The crude material was purified by chromatography through a Redi-Sep pre-packed silica gel column, eluting with a gradient of 0% to 40% EtOAc in heptane, to provide tert-butyl 4-(3-methyl-5-(oxetan-3-yl)isothiazol-4-yl)piperidine-1-carboxylate. m/z (ESI): 339.1 (M+H)⁺.

The intermediates in the table below were prepared in a fashion similar to that described above for intermediate B-46. Synthesis of the building blocks is either described above or from commercial sources.

| | | | m/z (ESI): |
|---|---|---|---|
| Int. # | Chemical Structure | Name | (M + H)⁺ |
| B-47 | 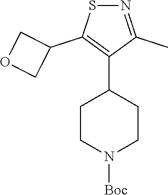 [SMILES: Cc1nsc(C2COC2)c1C1CCN(C(=O)OC(C)(C)C)CC1] | tert-butyl 4-(3-methyl-5-(oxetan-3-yl)isothiazol-4- yl)piperidine-1-carboxylate | 339.1 |
| B-48 | 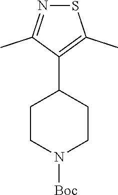 | tert-butyl 4-(3,5-dimethylisothiazol-4- yl)piperidine-1-carboxylate | 297.2 |
| B-59 | 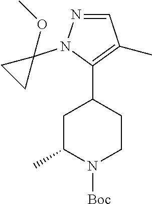 [SMILES: COC1(n2ncc(C)c2C2CCN(C(=O)OC(C)(C)C)[C@H](C)C2)CC1] | tert-butyl (2R)-4-(1-(1-methoxycyclopropyl)-4- methyl-1H-pyrazol-5-yl)-2-methylpiperidine-1- carboxylate | 350.0 |
| B-60 | 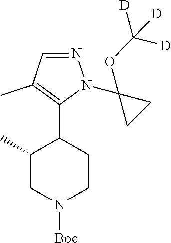 [SMILES: [2H]C([2H])([2H])OC1(n2ncc(C)c2C2CCN(C(=O)OC(C)(C)C)C[C@@H]2C)CC1] | tert-butyl (3R)-4-(1-(1-(methoxy-d₃)cyclopropyl)- 4-methyl-1H-pyrazol-5-yl)-3-methylpiperidine-1- carboxylate | 353.2 |
| B-61 | 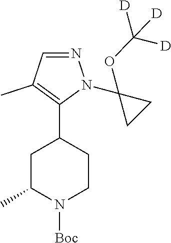 [SMILES: [2H]C([2H])([2H])OC1(n2ncc(C)c2C2CCN(C(=O)OC(C)(C)C)[C@H](C)C2)CC1] | tert-butyl (2R)-4-(1-(1-(methoxy-d₃)cyclopropyl)- 4-methyl-1H-pyrazol-5-yl)-2-methylpiperidine-1- carboxylate | 353.2 |
| B-62 | 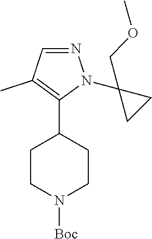 [SMILES: COCC1(n2ncc(C)c2C2CCN(C(=O)OC(C)(C)C)CC2)CC1] | tert-butyl 4-(1-(1-(methoxymethyl)cyclopropyl)-4- methyl-1H-pyrazol-5-yl)piperidine-1-carboxylate | 350.2 |
| B-63 | 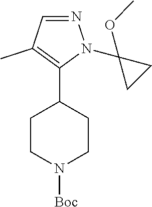 [SMILES: COC1(n2ncc(C)c2C2CCN(C(=O)OC(C)(C)C)CC2)CC1] | tert-butyl 4-(1-(1-methoxycyclopropyl)-4-methyl- 1H-pyrazol-5-yl)piperidine-1-carboxylate | 336.2 |
| B-64 | 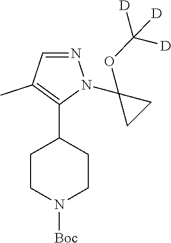 [SMILES: [2H]C([2H])([2H])OC1(n2ncc(C)c2C2CCN(C(=O)OC(C)(C)C)CC2)CC1] | tert-butyl 4-(1-(1-(methoxy-d3)cyclopropyl)-4- methyl-1H-pyrazol-5-yl)piperidine-1-carboxylate | 339.1 |
| B-65 | 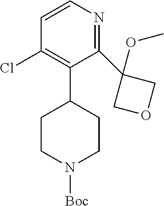 [SMILES: COC1(c2nccc(Cl)c2C2CCN(C(=O)OC(C)(C)C)CC2)COC1] | tert-butyl 4-(4-chloro-2-(3-methoxyoxetan-3- yl)479yridine-3-yl)piperidine-1-carboxylate | 381.2 |
| B-66 | 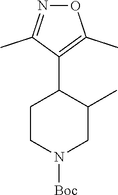 | tert-butyl 4-(3,5-dimethylisoxazol-4-yl)-3- methylpiperidine-1-carboxylate | 295.0 |
| B-67 | 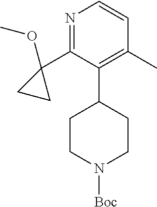 | tert-butyl 4-(2-(1-methoxycyclopropyl)-4- methylpyridin-3-yl)piperidine-1-carboxylate | 347.0 |
| B-68 | 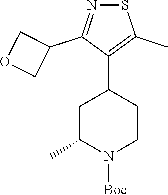 [SMILES: Cc1snc(C2COC2)c1C1CCN(C(=O)OC(C)(C)C)[C@H](C)C1] | tert-butyl (2R)-2-methyl-4-(5-methyl-3-(oxetan-3- yl)isothiazol-4-yl)piperidine-1-carboxylate | 353.1 |
| B-69 | 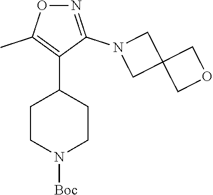 [SMILES: Cc1onc(N2CC3(COC3)C2)c1C1CCN(C(=O)OC(C)(C)C)CC1] | tert-butyl 4-(5-methyl-3-(2-oxa-6- azaspiro[3.3]heptan-6-yl)isoxazol-4-yl)piperidine- 1-carboxylate | 364.0 |
| B-70 | 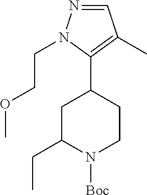 [SMILES: CCC1CC(c2c(C)cnn2CCOC)CCN1C(=O)OC(C)(C)C] | tert-butyl 2-ethyl-4-(1-(2-methoxyethyl)-4-methyl- 1H-pyrazol-5-yl)piperidine-1-carboxylate | 352.0 |
| B-71 | 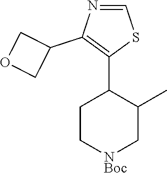 [SMILES: CC1CN(C(=O)OC(C)(C)C)CCC1c1scnc1C1COC1] | tert-butyl 3-methyl-4-(4-(oxetan-3-yl)thiazol-5- yl)piperidine-1-carboxylate | 339.2 |
| B-72 | 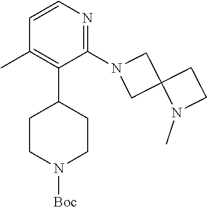 [SMILES: Cc1ccnc(N2CC3(CCN3C)C2)c1C1CCN(C(=O)OC(C)(C)C)CC1] | tert-butyl 4-(4-methyl-2-(1-methyl-1,6- diazaspiro[3.3]heptan-6-yl)480yridine-3- yl)piperidine-1-carboxylate | 387.1 |
| B-73 | 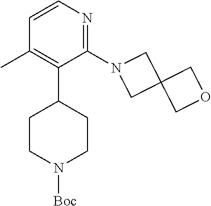 [SMILES: Cc1ccnc(N2CC3(COC3)C2)c1C1CCN(C(=O)OC(C)(C)C)CC1] | tert-butyl 4-(4-methyl-2-(2-oxa-6- azaspiro[3.3]heptan-6-yl)480yridine-3- yl)piperidine-1-carboxylate | 374.0 |
| B-74 | 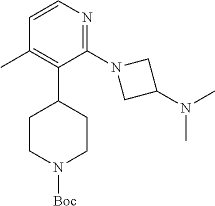 | tert-butyl 4-(2-(3-(dimethylamino)480yridine480- 1-yl)-4-methylpyridin-3-yl)piperidine-1- carboxylate | 375.3 |
| B-75 | 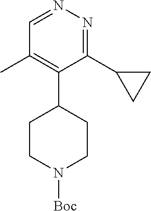 [SMILES: Cc1cnnc(C2CC2)c1C1CCN(C(=O)OC(C)(C)C)CC1] | tert-butyl 4-(3-cyclopropyl-5-methylpyridazin-4- yl)piperidine-1-carboxylate | 318.2 |
| B-76 | 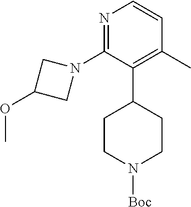 [SMILES: COC1CN(c2nccc(C)c2C2CCN(C(=O)OC(C)(C)C)CC2)C1] | tert-butyl 4-(2-(3-metboxyazetidin-1-yl)-4- methylpyridin-3-yl)piperidine-1-carboxylate | 362.0 |
| B-77 | 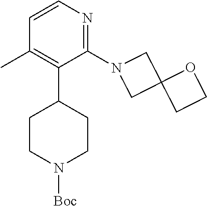 [SMILES: Cc1ccnc(N2CC3(CCO3)C2)c1C1CCN(C(=O)OC(C)(C)C)CC1] | tert-butyl 4-(4-methyl-2-(1-oxa-6- azaspiro[3.3]heptan-6-yl)481yridine-3- yl)piperidine-1-carboxylate | 374.2 |
| B-78 | 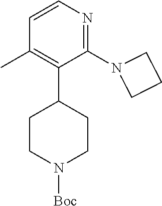 [SMILES: Cc1ccnc(N2CCC2)c1C1CCN(C(=O)OC(C)(C)C)CC1] | tert-butyl 4-(2-(azetidin-1-yl)-4-methylpyridin-3- yl)piperidine-1-carboxylate | 332.0 |
| B-79 | 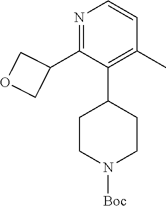 [SMILES: Cc1ccnc(C2COC2)c1C1CCN(C(=O)OC(C)(C)C)CC1] | tert-butyl 4-(4-methyl-2-(oxetan-3-yl)pyridin-3- yl)piperidine-1-carboxylate | 333.2 |
| B-80 | 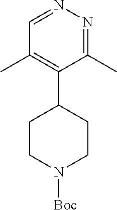 | tert-butyl 4-(3,5-dimethylpyridazin-4-yl)piperidine- 1-carboxylate | 292.2 |
| B-81 | 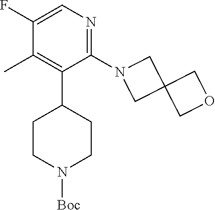 [SMILES: Cc1c(F)cnc(N2CC3(COC3)C2)c1C1CCN(C(=O)OC(C)(C)C)CC1] | tert-butyl 4-(4-fluoro-2-(2-oxa-6- azaspiro[3.3]heptan-6-yl)pyridin-3-yl)piperidine-1- carboxylate | 392.1 |
| B-82 | 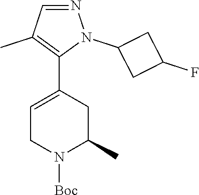 [SMILES: Cc1cnn(C2CC(F)C2)c1C1=CCN(C(=O)OC(C)(C)C)[C@H](C)C1] | tert-butyl (R)-4-(1-(3-fluorocyclobutyl)-4-methyl- 1H-pyrazol-5-yl)-2-methyl-3,6-dihydropyridine- 1(2H)-carboxylate | 350.2 |

### SFC Conditions for Chiral Separation

| | | | Racemic SM \| separation |
|---|---|---|---|
| Int.# | Chemical Structure | Name | condition |
| B-71-1 | 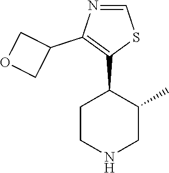 [SMILES: C[C@@H]1CNCC[C@H]1c1scnc1C1COC1] | 5-((3S,4R)-3-methylpiperidin-4- yl)-4-(oxetan-3-yl)thiazole | B-71 \| Chiralcel OD 250 x 30 mm, 3 μm column using a mobile phase of 10-30% MeOH in liquid CO₂ with a flow rate of 70 mL/min |
| | I^{st}-eluting isomer | | |
| B-71-2 | 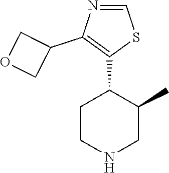 [SMILES: C[C@H]1CNCC[C@@H]1c1scnc1C1COC1] | 5-((3R,4S)-3-methylpiperidin-4- yl)-4-(oxetan-3-yl)thiazole | B-71 \| Chiralcel OD 250 x 30 mm, 3 μm column using a mobile phase of 10-30% MeOH in liquid CO₂ with a flow rate of 70 mL/min |
| | 2^{nd}-eluting isomer | | |
| B-82-1 | 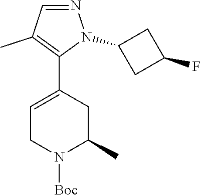 [SMILES: Cc1cnn([C@H]2C[C@H](F)C2)c1C1=CCN(C(=O)OC(C)(C)C)[C@H](C)C1] | tert-butyl (R)-4-(1-((1s,3S)-3- fluorocyclobutyl)-4-methyl-1H- pyrazol-5-yl)-2-methyl-3,6- dihydropyridine-1(2H)- carboxylate | B-82 \| a coupled Lux Cellulose-2, 2 x 25 cm 5 μm + 2 x 15 cm column with a mobile phase of 10% MeOH using a flowrate of 100 mL/min |
| | 1^{st}-eluting isomer | | |
| B-82-2 | 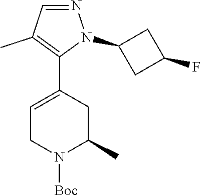 [SMILES: Cc1cnn([C@H]2C[C@@H](F)C2)c1C1=CCN(C(=O)OC(C)(C)C)[C@H](C)C1] | tert-butyl (R)-4-(1-((1s,3S)-3- fluorocyclobutyl)-4-methyl-1H- pyrazol-5-yl)-2-methyl-3,6- dihydropyridine-1(2H)- carboxylate | B-82 \| a coupled Lux Cellulose-2, 2 x 25 cm 5 μm + 2 x 15 cm column with a mobile phase of 10% MeOH using a flowrate of 100 mL/min |
| | 2^{nd}-eluting isomer | | |

### (3R,4S)-4-(1,4-Dimethyl-1H-pyrazol-5-yl)-3-methylpiperidine and (3S,4R)-4-(1,4-dimethyl-1H-pyrazol-5-yl)-3-methylpiperidine (Intermediate B-50)

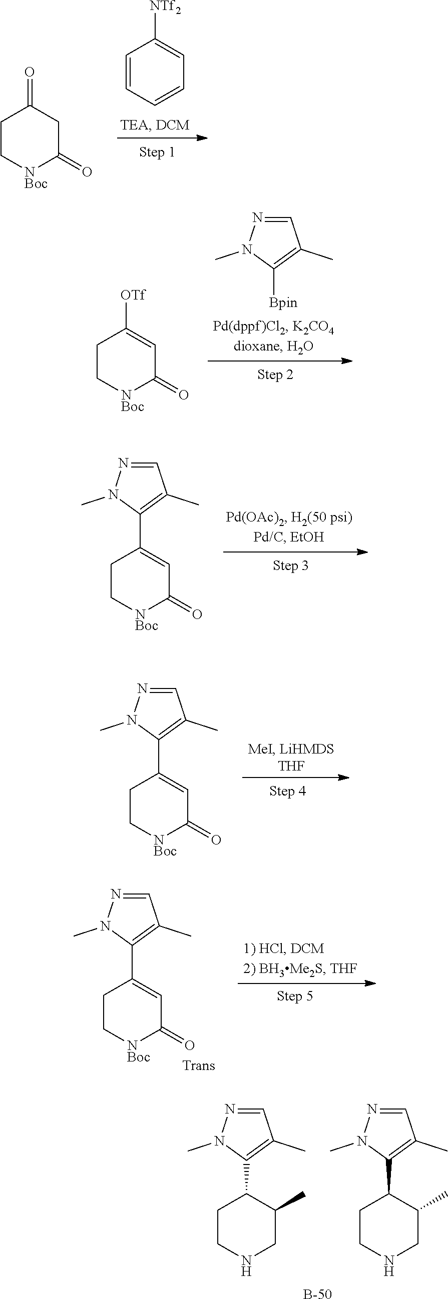

Step 1. To a solution of tert-butyl 2,4-dioxopiperidine-1-carboxylate (5.0 g, 23.45 mmol) in DCM (150 mL) was added TEA (6.06 g, 46.9 mmol). After addition, the mixture was stirred at 0° C. for 1 h, and then 1,1,1-trifluoro-N-phenyl-N-((trifluoromethyl) sulfonyl) methanesulfonamide (9.21 g, 25.8 mmol) was added at 0° C. The resulting mixture was stirred at 0° C. for 3 h, diluted with H₂O (100 mL), and extracted with EtOAc (50 mL×3). The combined organic extracts were dried over Na₂SO₄, filtered, and concentrated under reduced pressure. The residue was purified by column chromatography (SiO₂, 0% to 25% EtOAc in pet ether) to give tert-butyl 6-oxo-4-(((trifluoromethyl) sulfonyl)oxy)-3,6-dihydropyridine-1-(2H)-carboxylate.

Step 2. A mixture of tert-butyl 6-oxo-4-(((trifluoromethyl) sulfonyl)oxy)-3,6-dihydropyridine-1(2H)-carboxylate (22.5 g, 65.2 mmol), 1,4-dimethyl-5-(4,4,5,5-tetramethyl-1,3,2-dioxaborolan-2-yl)-1H-pyrazole (15.92 g, 71.7 mmol), K₂CO₃ (27.0 g, 195 mmol), and Pd(dppf)Cl₂ (4.77 g, 6.52 mmol) in 1,4-dioxane (400 mL) and H₂O (40 mL) was degassed and purged with N₂ 3 times, and the mixture was stirred at 95° C. for 2 h under N₂ atmosphere. The reaction mixture was diluted with H₂O (800 mL) and extracted with EtOAc (300 mL×3). The combined organic extracts were dried over Na₂SO₄, filtered, and concentrated under reduced pressure. The residue was purified by column chromatography (SiO₂. 0% to 25% EtOAc in pet ether) to give tert-butyl 4-(1,4-dimethyl-1H-pyrazol-5-yl)-6-oxo-3,6-dihydropyridine-1-(2H)-carboxylate.

Step 3. To a solution of tert-butyl 4-(1,4-dimethyl-1H-pyrazol-5-yl)-6-oxo-3,6-dihydropyridine-1(2H)-carboxylate (10.0 g, 34.3 mmol) in EtOH (100 mL) was added Pd/C (5.0 g, 34.3 mmol) and Pd(OAc)₂ (2.312 g, 10.30 mmol) under Ar atmosphere. The suspension was degassed and purged with H₂ (3 times). The mixture was stirred under H₂ (50 psi) at 75° C. for 12 h. The reaction mixture was diluted with H₂O (500 mL) and extracted with EtOAc (200 mL×3). The combined organic extracts were dried over Na₂SO₄, filtered, and concentrated under reduced pressure. The residue was purified by column chromatography (SiO₂, 0% a to 20% EtOAc in pet ether) to give tert-butyl 4-(1,4-dimethyl-1H-pyrazol-5-yl)-2-oxopiperidine-1-carboxylate.

Step 4. A solution of tert-butyl 4-(1,4-dimethyl-1H-pyrazol-5-yl)-2-oxopiperidine-1-carboxylate (6.0 g, 20.45 mmol) in THF (108 mL) was cooled to −78° C. Then LiHMDS (23.52 mL, 23.52 mmol, 1.0 M in THF) was added dropwise and the mixture was stirred at −78° C. for 0.5 h. Then iodomethane (1.53 mL, 24.54 mmol) was added dropwise at −78° C., and the resulting mixture was stirred at 20° C. for 20 h. The reaction mixture was quenched by the addition sat. aq. NH₄Cl (200 mL) at 20° C., diluted with H₂O (100 mL), and extracted with EtOAc (100 mL×3). The combined organic extracts were washed with brine (60 mL×2), dried over Na₂SO₄, filtered, and concentrated under reduced pressure. The residue was purified by column chromatography (SiO₂, 20% to 35% EtOAc in pet ether) to give tert-butyl (3R*,4S*)-4-(1,4-dimethyl-1H-pyrazol-5-yl)-3-methyl-2-oxopiperidine-1-carboxylate.

Step 5. To a 500 mL round-bottomed flask was added tert-butyl (3R,4S)-4-(1,4-dimethyl-1H-pyrazol-5-yl)-3-methyl-2-oxopiperidine-1-carboxylate (4.0 g, 13.01 mmol) in DCM (100 mL) at 0° C. Then aq. HCl (4 M, 32.5 mL, 130 mmol) was added slowly to the vigorously stirred reaction. The mixture was stirred at 20° C. for 1 h. The reaction was then carefully concentrated to a yellow foam, suspended in THF (100 mL), and borane dimethyl sulfide complex (3.9 mL, 39.0 mmol) was added. The mixture was then heated to 70° C. for 16 h. The reaction was cooled to 20° C., and HCl (4 M, 32.5 mL, 130 mmol) was added dropwise, the reaction was then heated to 70° C. for 1 h. The reaction was then cooled to 20° C. and the mixture was slowly poured into a separatory funnel containing 6N aqueous NaOH (160 mL), and then diluted with H₂O (400 mL) and extracted with DCM (200 mL×3). The combined organic extracts were washed with brine (100 mL×2), dried over Na₂SO₄, filtered, and concentrated under reduced pressure to give (3R*, 4S*)-4-(1,4-dimethyl-1H-pyrazol-5-yl)-3-methylpiperidine. m/z (ESI): 334.2 (M+H)⁺. ¹H NMR (400 MHz, chloroform-d) δ ppm 7.20 (d, J=4.8 Hz, 1H), 3.82 (s, 3H), 3.14-3.18 (m, 1H), 2.65-2.75 (m, 1H), 2.40-2.50 (m, 1H), 2.34 (t, J=11.2 Hz, 1H), 2.07 (s, 3H), 1.92-2.05 (m, 1H), 1.75-1.90 (m, 1H), 1.66-1.72 (m, 2H), 0.69 (d, J=6.8 Hz, 3H).

### Tert-Butyl 4-(4-(3-fluorooxetan-3-yl)thiazol-5-yl)piperidine-1-carboxylate (Intermediate B-51)

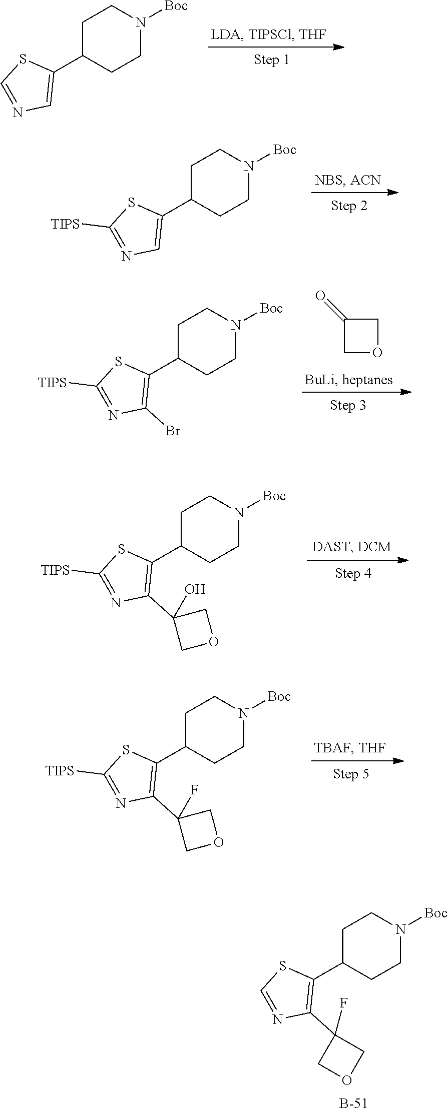

Step 1. To a solution of tert-butyl 4-(thiazol-5-yl) piperidine-1-carboxylate (15.0 g, 55.9 mmol) in THF (150 mL) was added LDA (2 M solution in THF, 84 mL, 168 mmol) dropwise at −78° C., over 0.5 h. After addition, the mixture was stirred at −78° C. for 0.5 h, and then chlorotriisopropylsilane (21.55 g, 112 mmol) was added dropwise at −78° C. The resulting mixture was stirred at 20° C. for 8 h, quenched by addition of H₂O 1 L) at 0° C., and extracted with EtOAc (500 mL×3). The combined organic extracts were dried over Na₂SO₄, filtered, and concentrated under reduced pressure. Purification by column chromatography (SiO₂, 0% to 10% EtOAc in pet ether) gave tert-butyl 4-(2-(triisopropylsilyl) thiazol-5-yl) piperidine-1-carboxylate.

Step 2. To a solution of ten-butyl 4-(2-(triisopropylsilyl) thiazol-5-yl) piperdine-1-carboxylate (6.0 g, 14.13 mmol) in CH₃CN (60 mL was added NBS (5.03 g, 28.3 mmol). Then the mixture was stirred at 30° C. for 12 h, quenched by the addition of H₂O (30 mL) at 0° C., and then extracted with EtOAc (200 mL, 2). The combined organic extracts were dried over Na₂SO₄, filtered, and concentrated under reduced pressure. The residue was purified by column chromatography (SiO₂, 1% to 3% EtOAc in pet ether) to give tert-butyl 4-(4-bromo-2-(triisopropylsilyl) thiazol-5-yl) piperidine-1-carboxylate.

Step 3. To a solution of tert-butyl 4-(4-bromo-2-(triisopropylsilyl) thiazol-5-yl) piperidine-1-carboxylate (4.5 g, 8.94 mmol) in heptane (90 mL) was added n-BuLi (2.5 M in hexanes, 14.31) mL, 35.7 mmol, 1.6 M in THF) dropwise at −50° C. After addition, the mixture was stirred at −50° C. for 1 h. and then oxetan-3-one (12.88 g, 179 mmol) was added dropwise at −50° C. The resulting mixture was stirred at 20° C. for 1 h, quenched by the addition of sat. aq. NH₄Cl (100 mL) at 0° C., and extracted with EtOAc (100 mL×2). The combined organic extracts were dried over Na₂SO₄, filtered, and concentrated. The residue was purified by column chromatography (SiO₂, 0% to 30% EtOAc in pet ether) to give tert-butyl 4-(4-(3-hydroxyoxetan-3-yl)-2-(triisopropylsilyl) thiazol-5-yl) piperidine-1-carboxylate.

Step 4. To a solution of ten-butyl 4-(4-(3-hydroxyoxetan-3-yl)-2-(triisopropylsilyl) thiazol-5-yl) piperidine-1-carboxylate (3.3 g, 6.64 mmol) in DCM (60 mL) was added DAST (1.755 mL, 13.29 mmol) dropwise at −10° C. Then the mixture was stirred at −10° C. for 1 h, quenched by addition of H₂O (100 mL) at 0° C., and then extracted with DCM (100 mL×2). The combined organic extracts were dried over Na₂SO₄, filtered, and concentrated wider reduced pressure. The crude product was triturated with 2% EtOAc in pet ether (30 mL) at 20° C. for 20 min to provide tert-butyl 4-(4-(3-fluorooxetan-3-yl)-2-(triisopropylsilyl) thiazol-5-yl) piperidine-1-carboxylate.

Step 5. To a solution of tert-butyl 4-(4-(3-fluorooxetan-3-yl)-2-(triisopropylsilyl) thiazol-5-yl) piperidine-1-carboxylate (2.5 g, 5.01 mmol) in THF (60 mL) was added TBAF (1 M in THF, 17.54 mL, 17.54 mmol). The mixture was stirred at 20° C. for 1 h and concentrated under reduced pressure. The residue was purified by column chromatography (SiO₂, 0% to 30% EtOAc in pet ether) to give tert-butyl 4-(4-(3-fluorooxetan-3-yl) thiazol-5-yl) piperidine-1-carboxylate. m/z (ESI): 343.0 (M+H)⁺. ¹H NMR (400 MHz, chloroform-d) δ ppm 8.66 (s, 1H), 5.12-5.27 (m, 2H), 5.05-5.11 (m, 2H), 4.22-4.24 (m, 2H), 3.05-3.12 (m, 1H), 2.75-2.82 (m, 2H), 1.91 (d, J=13.2 Hz, 2H), 1.51-1.60 (m, 2H), 1.48 (s, 9H).

### 4-(4-Fluoro-1-(oxetan-3-yl)-1H-pyrazol-5-yl)piperidine (Intermediate B-52)

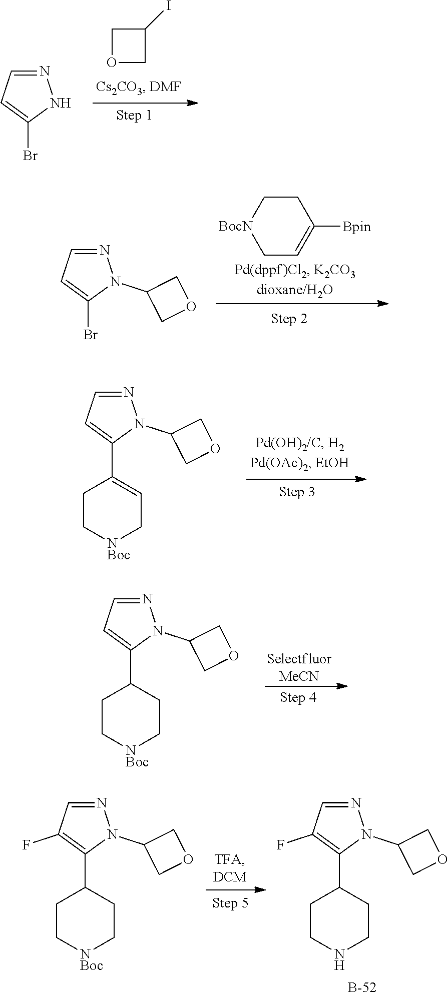

Step 1. A mixture of 5-bromo-1H-pyrazole (10.0 g, 68.0 mmol, WuXi AppTec, China), Cs₁CO₃ (28.8 g, 88 nmol), and 3-iodooxetane (13.77 g, 74.8 nmol, Accela ChemaBio, China) in DMF (300 mL) was degassed and purged with N₂ (3×). The reaction mixture was stirred at 110° C. for 18 h under a N₂ atmosphere. The reaction mixture was diluted with H₂O (300 mL) and extracted with EtOAc (200 mL×3). The combined organic extracts were dried over Na₂SO₄, filtered, and concentrated under reduced pressure. The residue was purified by column chromatography (SiO₂, 0% to 5% EtOAc in pet ether) to give 5-bromo-1-(oxetan-3-yl)-1H-pyrazole.

Step 2. A mixture of K₂CO₃ (17.02 g, 123 mmol), Pd(dppf)Cl₂ (4.50 g, 6.16 mmol), 5-bromo-1-(oxetan-3-yl)-1H-pyrazole (12.5 g, 61.6 mmol), and tert-butyl 4-(1-(oxetan-3-yl)-1H-pyrazol-5-yl)-3,6-dihydropyridine-1(2H)-carboxylate in 1,4-dioxane (400 mL) and H₂O (40 mL) was degassed and purged with N₂ (3×) at 20° C. The reaction mixture was stirred at 100° C. for 1 h under a N₂ atmosphere. The reaction mixture was diluted with 1H₂O (500 mL) and extracted with EtOAc (3 L×3). The combined organic extracts were dried over Na₂SO₄, filtered, and concentrated under reduced pressure. The residue was purified by column chromatography (SiO₂, 0% to 50% EtOAc in pet ether) to give tert-butyl 4-(1-(oxetan-3-yl)-1H-pyrazol-5-yl)-3,6-dihydropyridine-1 (2H)-carboxylate.

Step 3. To a mixture of Pd(OH)₂/C (6.90 g, 9.82 mmol) and Pd(OAc)₂ (2.21 g, 9.82 mmol) in EtOH (180 mL) was added tert-butyl 4-(1-(oxetan-3-yl)-1H-pyrazol-5-yl)-3,6-dihydropyridine-1(2H)-carboxylate (15 g, 49.1 mmol) under Ar atmosphere. The suspension was degassed, purged with H₂ (3×), and stirred under H₂ (40 psi) at 25° C. for 12 h. The reaction mixture was filtered and concentrated under reduced pressure to give tert-butyl 4-(1-(oxetan-3-yl)-1H-pyrazol-5-yl)piperidine-1-carboxylate.

Step 4. A mixture of tert-butyl 4-O-(oxetan-3-yl)-1H-pyrazol-5-yl) piperidine-1-carboxylate (4.55 g, 14.80 mmol) and Selectfluor (15.73 g, 44.4 mmol) in CH₃CN (100 mL) was degassed and purged with N₂ (3×) at 20° C. The reaction mixture was stirred at 50° C. for 2 h under N₂ atmosphere. The solvent was removed under vacuum and the residue was purified by prep-HPLC (column. Phenomenex Luna C18 (250×70 mm, 15 μm, H₂O (HCl)-ACN, 33-63%, 20 min) to give tert-butyl 4-(4-fluoro-1-(oxetan-3-yl)-1H-pyrazol-5-yl) piperdine-1-carboxylate.

Step 5. To a mixture of tert-butyl 4-(4-fluoro-1-(oxetan-3-yl)-1H-pyrazol-5-yl) piperidine-1-carboxylate (0.8 g, 2.46 mmol) in DCM (20 mL), was added TFA (2 mL) dropwise. The mixture was stirred at 20° C. for 3 h under N₂ atmosphere. The reaction mixture was adjusted to pH=7 with sat. aq. NaHCO₃, filtered, and concentrated under reduced pressure. The residue was purified by column chromatography (SiO₂, 0% to 100% EtOAc in pet ether) to give 4-(4-fluoro-1-(oxetan-3-yl)-1H-pyrazol-5-yl) piperidine. m/z (ESI): 226.1 (M+H)⁺. ¹H NMR (400 MHz, CD₃OD) δ ppm 7.49 (d, J=4.0 Hz, 1H), 5.60-5.67 (m, 1H), 5.08 (t, J=6.4 Hz, 2H), 4.98 (t, J=7.2 Hz, 2H), 3.32-3.38 (m, 2H), 2.94-3.06 (m, 3H), 1.96-2.03 (m, 4H). Note: The NH proton is not observed.

### (3S,4R)-3-Methyl-4-(4-methyl-1-((S)-tetrahydrofuran-3-yl)-1H-pyrazol-5-yl)piperidine (Intermediate B-83)

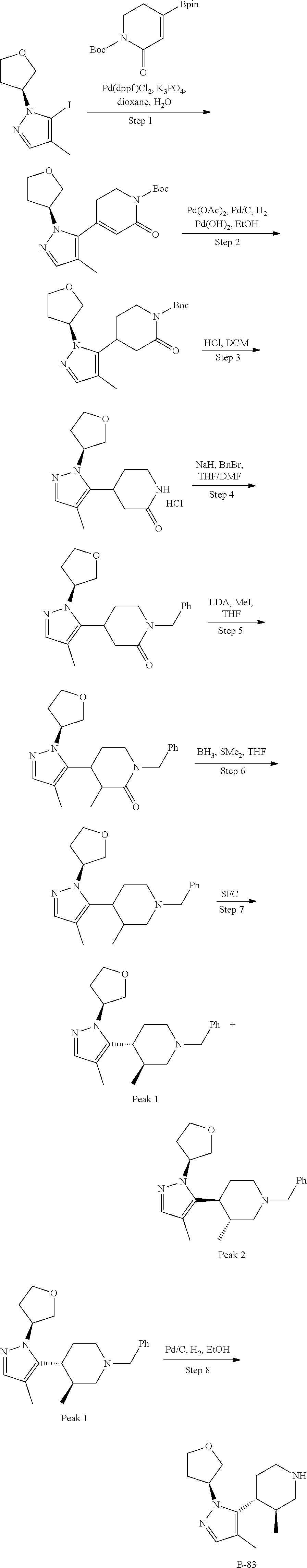

Step 1. To a vessel % as added (S)-5-iodo-4-methyl-1-(tetrahydrofuran-3-yl)-1H-pyrazole (100 g, 360 mmol), K₃PO₄ (229 g, 1079 mmol) and tert-butyl 6-oxo-4-(4,4,5,5-tetramethyl-1,3,2-dioxaborolan-2-yl)-3,6-dihydropyridine-1(2H)-carboxylate (139 g, 432 mmol) in 1,4-dioxane (2000 mL) and H₂O (200 mL). The reaction mixture was purged with N₂ for 10 min, Pd(dppf)Cl₂ DCM (29.4 g, 36.0 mmol) was added and the reaction mixture was purged with N₂ for 5 min and then heated to 100° C. for 4 h. The reaction mixture was cooled to rt, quenched with H₂O (300 mL), and extracted with DCM (3×100 mL). The organic layer was washed with H₂) (100 mL), dried over Na₂SO₄, filtered and concentrated under reduced pressure. The crude residue was purified by Redi-Sep pre-packed silica gel column, eluting with 0-50% EtOAc in hexanes to give tert-butyl 4-(4-methyl-1-((S)-tetrahydrofuran-3-yl)-1H-pyrazol-5-yl)-2-oxopiperidine-1-carboxylate which was used directly in the next step. m/z (ESI): 348.2 (M+H)⁺.

Step 2. To a solution of tert-butyl 4-(4-methyl-1-((S)-tetrahydrofuran-3-yl)-1H-pyrazol-5-yl)-2-oxopiperidine-1-carboxylate (5 g, 14.39 mmol, 1.0 equiv) in EtOH (100 mL) was added palladium(II) acetate (2.5 g, 11.14 mmol, 0.774 equiv) and stirred for 5 min. Pd—C (10% wt./wt., 5 g) and palladium hydroxide on carbon (10% wt./wt., 5 g) were added next under N₂ atmosphere. The reaction mixture was stirred under hydrogen atmosphere (14 psi) at rt for 3 h. The reaction mixture was filtered through a celite bed and washed with EtOH (100 mL) and DCM (100 mL). The filtrate was concentrated under reduced pressure to give tert-butyl 4-(4-methyl-1-(S)-tetrahydrofuran-3-yl)-1H-pyrazol-5-yl)-2-oxopiperidine-1-carboxylate. m/z (ESI): 350.3 (M+H)⁺.

Step 3. To a stirred solution of tert-butyl 4-(4-methyl-1-((S)-tetrahydrofuran-3-yl)-1H-pyrazol-5-yl)-2-oxopiperidine-1-carboxylate (100 g, 286 mmol) in DCM (1000 mL) was added HCl (4 M in 1,4-dioxane) (215 mL, 859 mmol) at 0° C. The reaction mixture was stirred at rt for 2 h. DCM was decanted from reaction mass and subjected to workup to provide 4-(4-methyl-1-((S)-tetrahydrofuran-3-yl)-1H-pyrazol-5-yl)piperidin-2-one hydrochloride. m/z (ESI): 250.1 (M+H)⁺.

Step 4. To a vessel was added 4-(4-methyl-1-((S)-tetrahydrofuran-3-yl)-1H-pyrazol-5-yl)piperidin-2-one hydrochloride (80 g, 280 mmol) in DMF (8010 mL). Benzyl bromide (40.0 mL, 336 mmol) was added at rt followed by the addition of NaH (60% dispersion in mineral oil) (44.8 g, 1120 mmol) at 0° C. The reaction mixture was warm to rt and stirred for 2 h. The reaction mixture slowly poured into ice cold H₂O (1000 mL), extracted with EtOAc (2×700 mL) and DCM (2×500 mL). The combined organic layer was dried over Na₂SO₄, filtered and concentrated. The crude compound was purified by silica gel chromatography, eluting from 70 to 90% EtOAc in pet ether to give 1-benzyl-4-(4-methyl-1-((S)-tetrahydrofuran-3-yl)-1H-pyrazol-5-yl)piperidin-2-one. m/z (ESI): 340.1 (M+H)⁺.

Step 5. To a vessel was added 1-benzyl-4-(4-methyl-1-((S)-tetrahydrofuran-3-yl)-1H-pyrazol-5-yl)piperidin-2-one (56 g, 165 mmol) and THF (1120 mL). The solution was cooled to −78° C., and LDA (2 M solution in THF, 124 mL, 247 mmol) added dropwise. After stirring for 5 min at −78° C. MeI (11.35 mL, 181 mmol) was added at −78° C., and the solution was allowed to warm to rt and stirred for 30 min. The reaction was quenched with NH₄Cl (1500 mL) while cooling in an ice bath. The extracts were separated, and the aqueous layer was extracted with EtOAc (2×500 mL). The combined organic layer was dried over Na₂SO₄, filtered and concentrated. The crude material was purified by silica gel chromatography using 50-60% EtOAc in hexane to give 1-benzyl-3-methyl-4-(4-methyl-1-((S)-tetrahydrofuran-3-yl)-1H-pyrazol-5-yl)piperidin-2-one. m/z (ESI): 354.3 (M+H)⁺.

Step 6. To a vessel was added 1-benzyl-3-methyl-4-(4-methyl-1-((S)-tetrahydrofuran-3-yl)-1H-pyrazol-5-yl)piperidin-2-one (53 g, 150 mmol) in THF (1325 mL). The solution was cooled in an ice bath and borane tetrahydrofuran complex (1.0 M in THF, 450 mL, 450 mmol) was added dropwise. The solution was stirred at rt for 1 h. The reaction was slowly quenched with MeOH (1500 mL). After quenching, the reaction mixture was stirred at reflux temperature (70° C.) for 16 h. The volatiles were evaporated to afford the crude product, which was used for the next step directly. m/z (ESI): 340.2 (M+H)⁺.

Step 7. 1-Benzylbenzyl-3-methyl-4-(4-methyl-1-((S)-tetrahydrofuran-3-yl)-1H-pyrazol-5-yl)piperidine (53 g, 156 mmol) was purified by SFC with a Chiralpak IG (250×20.0) mm, 5μ, column using 50% (1:1) ACN:MeOH in liquid CO₂ to provide:
Peak-1. (3S,4R)-1-benzyl-3-methyl-4-(4-methyl-1-((S)-tetrahydrofuran-3-yl)-1H-pyrazol-5-yl)piperdine. ¹H NMR (401 MHz, DMSO-d₆): δ 7.38-7.30 (m, 4H), 7.26 (tt, J=6.9, 2.5 Hz, 1H), 7.19 (s, 1H), 5.12-5.00 (m, 1H), 3.98 (dt, J=25.5, 7.7 Hz, 2H), 3.82 (td, J=7.8, 6.0 Hz, 1H), 3.71 (dd, J=8.6, 5.0 Hz, 1H), 3.51 (s, 2H), 2.89 (dd, J=10.6, 3.9 Hz, 2H), 2.46 (d, J=12.4 Hz, 1H), 2.31-2.14 (m, 2H), 2.10-1.95 (m, 5H), 1.87-1.70 (m, 2H), 1.64-1.57 (m, 1H), 0.59 (d, J=6.6 Hz, 3H).
Peak-2. (3R,4S)-1-benzyl-3-methyl-4-(4-methyl-1-((S)-tetrahydrofuran-3-yl)-1H-pyrazol-5-yl)piperidine.

¹H NMR (401 MHz, DMSO-d₆): δ 7.40-7.15 (m, 6H), 5.12-4.97 (m, 1H), 4.03-3.89 (m, 2H), 3.82 (td, J=7.7, 5.9 Hz, 1H), 3.69 (dd, J=8.7, 5.0 Hz, 1H), 3.51 (s, 2H), 3.42-3.28 (m, 6H), 2.89 (d, J=10.9 Hz, 2H), 2.45 (s, 15H), 2.35-2.11 (m, 3H), 2.10-1.94 (m, 5H), 1.91-1.65 (m, 2H), 1.57 (d, J=12.5 Hz, 1H), 1.47-1.21 (m, 2H), 0.61 (d, J=6.5 Hz, 3H).

Step 8. To a solution of (3S,4R)-1-benzyl-3-methyl-4-(4-methyl-1-((S)-tetrahydrofuran-3-yl)-1H-pyrazol-5-yl)piperidine (300 mg, 0.884 mmol) (Peak-1) in MeOH (15 mL) was added Pd/C (10% wt./wt., 150 mg). The reaction mixture was stirred under H₂ (14 psi) at rt for 8 h. The reaction mixture was filtered through a celite bed washed with 10% MeOH in DCM (100 mL). The filtrate was concentrated under reduced pressure to give (3S,4R)-3-methyl-4-(4-methyl-1-((S)-tetrahydrofuran-3-yl)-1H-pyrazol-5-yl)piperidine. m/z (ESI): 250.3 (M+H)⁺. ¹H NMR (400 MHz, DMSO-d₆) δ (ppm) 7.18 (s, 1H), 5.09-5.08 (m, 1H), 4.04-3.94 (m, 2H), 3.85-3.80 (m, 1H), 3.74-3.70 (m, 1H), 3.32 (s, 2H), 2.98-2.94 (m, 2H), 2.57-2.54 (m, 1H), 2.26-2.19 (m, 3H), 2.01 (s, 3H), 1.90-1.87 (m, 1H), 1.69-1.1.61 (m, 1H), 1.53-1.550 (m, 1H), 0.63-0.55 (m, 3H).

The intermediate in the table below was prepared in a fashion similar to that described above. Synthesis of the building blocks is either described above or from commercial sources.

| | | | m/z (ESI): |
|---|---|---|---|
| Int. # | Chemical Structure | Name | (M + H)⁺ |
| B-84 | 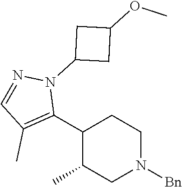 [SMILES: COC1CC(n2ncc(C)c2C2CCN(Cc3ccccc3)C[C@@H]2C)C1] | (3R)-1-benzyl-4-(1-(3-methoxycyclobutyl)-4- methyl-1H-pyrazol-5-yl)-3-methylpiperidine | 354.1 |

### 2-(3-Methoxytetrahydrofuran-3-yl)-4-methyl-3-((2R,4S)-2-methylpiperidin-4-yl)pyridine)

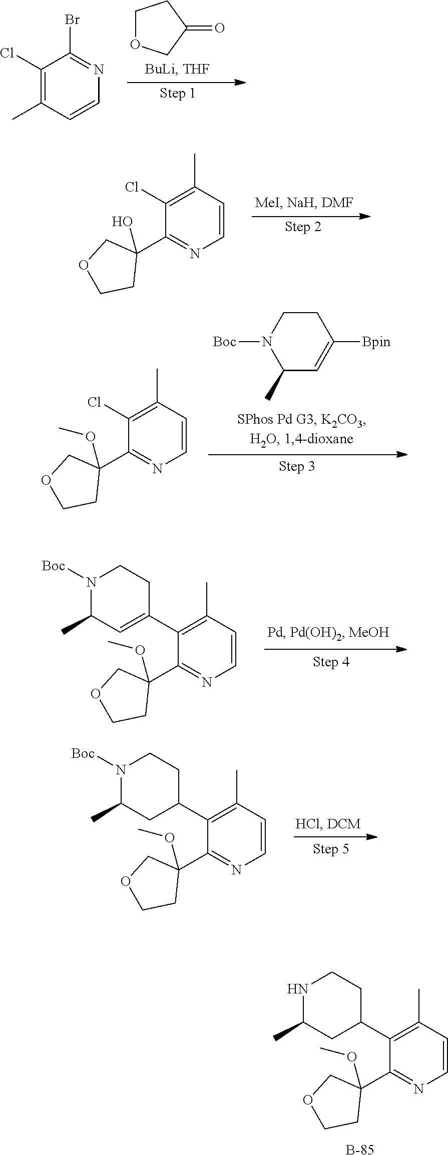

Step 1. To a solution of 2-bromo-3-chloro-4-methylpyridine (40 g, 194 mmol) in THF (1200 mL) at −78° C. was added n-BuLi (2.5 M in hexanes, 85 mL, 213 mmol) dropwise. The reaction mixture was stirred at −78° C. for 15 min. Then dihydrofuran-3(2H)-one (18.35 g, 213 mmol) in THF was added dropwise over 10 min and stirred at the same temperature for 15 mm. The reaction mixture was quenched with sat. aq. NH₄Cl and extracted with EtOAc (3×500 mL). The combined organic extracts were dried over Na₂SO₄, filtered, and concentrated in vacuo. The crude material was purified by chromatography, eluting with a gradient of 20% to 25% EtOAc in hexanes, to provide 3-(3-chloro-4-methylpyridin-2-yl)tetrahydrofuran-3-ol. ¹H NMR (DMSO-d₆, 401 MHz): δ (ppm) 8.31 (d, J=4.8 Hz, 1H), 7.37 (d, J=4.8 Hz, 1H), 5.59 (s, 1H), 4.16 (d, J=9.4 Hz, 1H), 4.07 (m, 1H), 3.95 (ddd, J=9.2, 7.9, 6.5 Hz, 1H), 3.86 (td, J=8.2, 3.5 Hz, 1H), 2.60 (dt, J=12.9, 8.8 Hz, 1H), 2.39 (s, 3H), 2.33 (dddd, J=12.9, 6.4, 3.4, 0.9 Hz, 1H)

Step 2. To a vessel was added at 3-(3-chloro-4-methylpyridin-2-yl)tetrahydrofuran-3-ol (18 g, 84 mmol) and NaH (4.04 g, 101 mmol) in DMF (360 mL). The reaction mixture was stirred at 0° C. for 15 min. Then MeI (5.79 mL, 93 mmol) was added at 0° C., and stirred for 1 h. Then ice water was added and the mixture was extracted with EtOAc. The organic extract was dried over Na₂SO₄, filtered, and concentrated in vacuo. The crude material was purified by chromatography, eluting with a gradient of 15% to 20% EtOAc in hexanes, to provide 3-chloro-2-(3-methoxytetrahydrofuran-3-yl)-4-methylpyridine. ¹H NMR (401 MHz, DMSO-d₆): δ (ppm) 8.37 (d, J=4.8 Hz, 1H), 7.43 (d, J=4.8 Hz, 1H), 4.27 (dd, J=9.9, 1.0 Hz, 1H), 4.10 (s, 1H), 3.85 (m, 2H), 2.88 (s, 3H), 2.60 (dt, J=13.3, 8.3 Hz, 2H), 2.41 (s, 3H)

Step 3. To a vessel was added 3-chloro-2-(3-methoxytetrahydrofuran-3-yl)-4-methylpyridine (16 g, 70.3 mmol) and tert-butyl (R)-6-methyl-4-(4,4,5,5-tetramethyl-1,3,2-dioxaborolan-2-yl)-3,6-dihydropyridine-1(2H)-carboxylate (31.2 g, 77 mmol) in 1,4-dioxane (320 mL) and H₂O (80 mL). To this potassium carbonate (29.1 g, 211 mmol) and SPhos Pd G3 (2.87 g, 3.51 mmol) was added. The reaction mixture was stirred at 90° C. for 6 h and allowed to warm to rt. The reaction mixture was diluted with H₂O and extracted with EtOAc. The organic extract was dried over Na₂SO₄, filtered, and concentrated in vacuo. The crude material was purified by chromatography, eluting with a gradient of 25% to 30% EtOAc in hexanes, to provide tert-butyl (6′R)-2-(3-methoxytetrahydrofuran-3-yl)-4,6′-dimethyl-3′,6′-dihydro-[3,4′-bipyridine]-1′(2′H)-carboxylate, which was dissolved in acetone:H₂O (1:1). To the reaction mixture was added sodium metaperiodate and the reaction mixture was stirred for 12 h. The reaction mixture was diluted with H₂O and extracted with EtOAc. The organic extract was dried over Na₂SO₄ The solution was filtered and concentrated in vacuo to give tert-butyl (6′R)-2-(3-methoxytetrahydrofuran-3-yl)-4,6′-dimethyl-3′,6′-dihydro-[3,4′-bipyridine]-1′(2′H)-carboxylate. ¹H NMR (401 MHz, DMSO-d₆): δ (ppm) 8.32 (m, 1H), 7.26 (dd, J=14.4, 4.8 Hz, 1H), 5.43 (m, 1H), 4.51 (m, 1H, 4.20 (m, 1H), 4.04 (m, 2H), 3.79 (ddt, J=19.8, 14.4, 8.9 Hz, 3H), 2.88 (d, J=2.4 Hz, 1H), 2.81 (d, J=3.1 Hz, 2H), 2.21 (d, J=3.0 Hz, 2H), 1.90 (m, 1H), 1.44 (m, 9H), 1.18 (m, 6H).

Step 4: To a stirred solution of tert-butyl (6′R)-2-(3-methoxytetrahydrofuran-3-yl)-4,6′-dimethyl-3′,6′-dihydro-[3,4′-bipyridine]-1′(2′H)-carboxylate (5.5 g, 14.16 mmol) in MeOH (110 mL) was added palladium hydroxide on carbon (10% wet) (1.9 g, 1.416 mmol) followed by Pd/C (10% wet) (1.5 g, 1.416 mmol). The reaction mixture was stirred at 40° C. for 5 days under H₂ (70 psi) pressure. The reaction was filtered through a celite bed and concentrated under vacuum to provide the crude material. The crude material was purified by silica gel chromatography, eluting with a gradient of 15% to 20% EtOAc in hexanes, to provide
Peak1: tert-butyl (2R,4R)-4-(2-(3-methoxytetrahydrofuran-3-yl)-4-methylpyridin-3-yl)-2-methylpiperidine-1-carboxylate. m/z (ESI): 391.2 (M+H)⁺: ¹H NMR (401 MHz, DMSO-d₆): δ (ppm) 8.22 (dd, J=4.8, 0.9 Hz, 1H), 7.16 (d, J=4.9 Hz, 1H), 4.09 (m, 2H), 3.80 (m, 3H), 3.59 (m, 1H), 3.21 (m, 2H), 2.90 (s, 3H), 2.60 (m, 1H), 2.44 (s, 3H), 2.30 (dddd, J=20.3, 12.6, 6.9, 4.7 Hz, 1H), 2.02 (tdd, J=13.8, 11.1, 3.5 Hz, 1H), 1.86 (m, 1H), 1.72 (m, 1H), 1.59 (tdd, J=13.6, 6.4, 3.3 Hz, 1H), 1.43 (s, 9H), 1.16 (d, J=6.2 Hz, 3H)
Peak-2: tert-butyl (2R,4S)-4-(2-(3-methoxytetrahydrofuran-3-yl)-4-methylpyridin-3-yl)-2-methylpiperidine-1-carboxylate. m/z (ESI): 391.2 (M+H)⁺; ¹H NMR (401 MHz, DMSO-d₆): δ (ppm) 8.22 (dd, J=6.9, 4.9 Hz, 1H), 7.12 (dd, J=4.9, 1.8 Hz, 1H), 4.43 (d, J=27.4 Hz, 1H), 4.19 (m, 1H), 3.90 (m, 4H), 3.17 (d, J=3.3 Hz, 2H), 2.93 (d, J=1.0 Hz, 4H), 2.74 (dt, J=13.2, 8.1 Hz, 1H), 2.42 (m, 4H), 2.28 (d, J=13.3 Hz, 1H), 1.99 (d, J=13.3 Hz, 1H), 1.43 (s, 10H), 1.21 (t, J=7.0 Hz, 3H)

Step 5. To a vessel was added tert-butyl (2R,4S)-4-(2-(3-methoxytetrahydrofuran-3-yl)-4-methylpyridin-3-yl)-2-methylpiperidine-1-carboxylate (0.8 g, 2.049 mmol) and HCl (2.56 mL, 10.24 mmol) in DCM (8.00 mL). The reaction mixture was stirred at r for 12 h. Then the reaction was concentrated under reduced pressure to provide 2-(3-methoxytetrahydrofuran-3-yl)-4-methyl-3-((2R)-2-methylpiperidin-4-yl)pyridine hydrochloride. m/z (ESI): 291.3 (M+H)⁺

### Tert-Butyl (2R,4S)-4-(2-(3-methoxyoxetan-3-yl)-4-methylpyridin-3-yl)-2-methylpiperidine-1-carboxylate (Intermediate B-86)

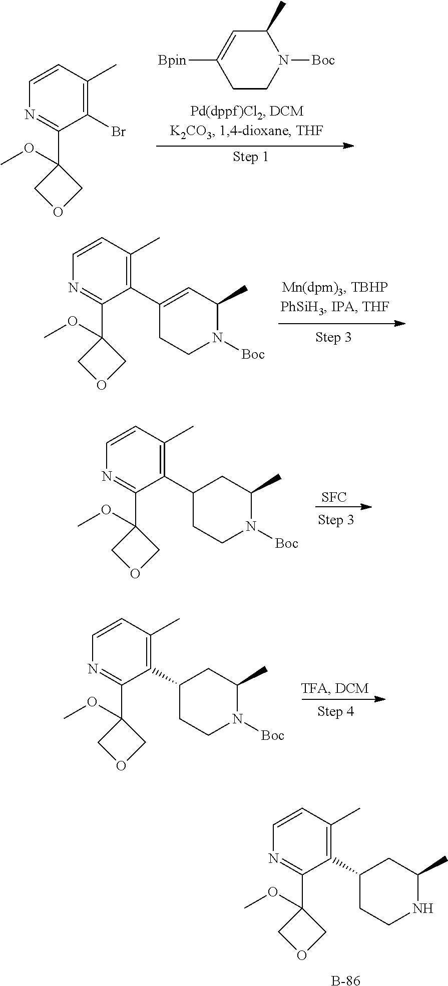

Step 1. To a solution of 3-bromo-2-(3-methoxyoxetan-3-yl)-4-methylpyridine (20 g, 77 mmol) in 1,4-dioxane (360 mL) and H₂O (40 mL) were added tert-butyl (R)-6-methyl-4-(4,4,5,5-tetramethyl-1,3,2-dioxaborolan-2-yl)-3,6-dihydropyridine-1(2H)-carboxylate (25.05 g, 77 mmol) and K₂CO₃ (32.1 g, 232 mmol). The reaction was degassed for 5 min with N₂ and Pd(dppf)Cl₂·DCM (3.16 g, 3.87 mmol) was added. The reaction mixture was heated to 90° C., and stirred for 16 h. The reaction mixture was allowed to cool to it. The reaction mixture was diluted with H₂O (100 mL) and extracted with EtOAc (2×100 mL). The combined organic extracts were dried over Na₂SO₄, filtered and concentrated in vacuo. The crude material was purified by silica gel column chromatography eluting with a gradient of 30-40% EtOAc in pet ether to provide tert-butyl (R)-2-(3-methoxyoxetan-3-yl)-4,6′-dimethyl-3′,6′-dihydro-[3,4′-bipyridine]-1′(2-H)-carboxylate. m/z (ESI): 375.3 (M+H)⁺

Step 2. To a stirred solution of tert-butyl (R)-2-(3-methoxyoxetan-3-yl)-4,6′-dimethyl-3′,6′-dihydro-[3,4′-bipyridine]-1′(2′H)-carboxylate (16 g, 42.7 mmol) in IPA (160 mL) and THF (160 mL) at 0° C. was added tris(2,2,6,6-tetramethyl-3,5-heptanedionato) manganese(III) (12.9 g, 21.36 mmol). Next, phenylsilane (26.3 mL, 214 mmol) followed by TBHP (5-6 M in decane) (42.7 mL, 214 mmol) was added dropwise at 0° C. under N₂ atmosphere. The reaction mixture was stirred at 0° C. for 30 min and 27° C. for 16 h. The reaction mixture was quenched with sat. aq. sodium metabisulfite solution (50 mL) and extracted with EtOAc (2×100 mL). The organic layer was washed with H₂O (100 mL), dried over Na₂SO₄, filtered and concentrated under reduced pressure. The crude residue was purified by column chromatography eluting with 0-60% EtOAc in hexanes to give tert-butyl (2R,4S)-4-(2-(3-methoxyoxetan-3-yl)-4-methylpyridin-3-yl)-2-methylpiperidine-1-carboxylate. m/z (ESI): 377.1 (M+H)⁺

Step 3. Crude tert-butyl (2R,4S)-4-(2(3-methoxyoxetan-3-yl)-4-methylpyridin-3-yl)-2-methylpiperidine-1-carboxylate was purified by SFC using a Chiralpak IC, 150×50 mm 5μ, column with a mobile phase of 15% MeOH in liquid CO₂ using a flowrate of 200 mL/min to provide tert-butyl (2R,4S)-4-(2-(3-methoxyoxetan-3-yl)-4-methylpyridin-3-yl)-2-methylpiperidine-1-carboxylate. m/z (ESI): 377.3 (M+H)⁺. ¹H NMR (400 MHz, DMSO-d₆): δ (ppm) 8.28 (d, J=4.9 Hz, 1H), 7.18 (d, J=5.0 Hz, 1H), 5.14 (d, J=32.4 Hz, 2H), 4.80 (dd, J=18.4, 7.1 Hz, 2H), 4.40 (d, J=27.1 Hz, 1H), 3.92 (dd, J=24.6, 13.9 Hz, 1H), 2.93 (s, 5H), 2.44 (s, 2H), 2.34-2.05 (m, 1H), 1.94 (d, J=13.2 Hz, 1H), 1.42 (s, 11H), 1.14 (d, J=18.9 Hz, 4H).

Step 4. To a solution of tert-butyl (2R,4S)-4-(2-(3-methoxyoxetan-3-yl)-4-methylpyridin-3-yl)-2-methylpiperidine-1-carboxylate (200 mg, 0.531 mmol) in DCM (4.00 mL) was added TFA (0.205 mL, 2.66 mmol) at 0° C. under N₂ atmosphere. The reaction mixture was stirred at rt for 2 h. The reaction mixture was concentrated under reduced pressure to afford 2-(3-methoxy oxetan-3-yl)-3-((2R,4S)-2-methylpiperidin-4-yl)pyridine, which was used as is without further purification. m/z (ESI): 277.3 (M+H)⁺.

The intermediate in the table below was prepared in a fashion similar to that described above. Synthesis of the building blocks is either described above or from commercial sources.

| | | | m/z (ESI): |
|---|---|---|---|
| Int. # | Chemical Structure | Name | (M + H)⁺ |
| B-87 | 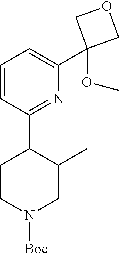 [SMILES: COC1(c2cccc(C3CCN(C(=O)OC(C)(C)C)CC3C)n2)COC1] | tert-butyl 4-(6-(3-methoxyoxetan-3-yl)pyridin-2- yl)-3-methylpiperidine-1-carboxylate | 363.0 |

### SFC Conditions for Chiral Separation

| | | | Racemic SM \| separation |
|---|---|---|---|
| Int.# | Chemical Structure | Name | condition |
| B-87-1 | 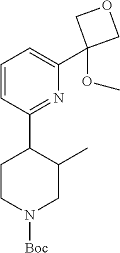 [SMILES: COC1(c2cccc(C3CCN(C(=O)OC(C)(C)C)CC3C)n2)COC1] | tert-butyl 4-(6-(trans-3- methoxyoxetan-3-yl)pyridin-2- yl)-3-methylpiperidine-1- carboxylate | B-87 \| Chiralpak AZ 21 x 250 mm 5 μm, column with a mobile phase of 10% MeOH using a flow rate of 150 mL/min |
| | trans | | |
| | 1^{st}-eluting isomer | | |

### 2-(3-Methoxyoxetan-3-yl)-4-methyl-3-((3S,4R)-3-methylpiperidin-4-yl)pyridine (Intermediate B-88)

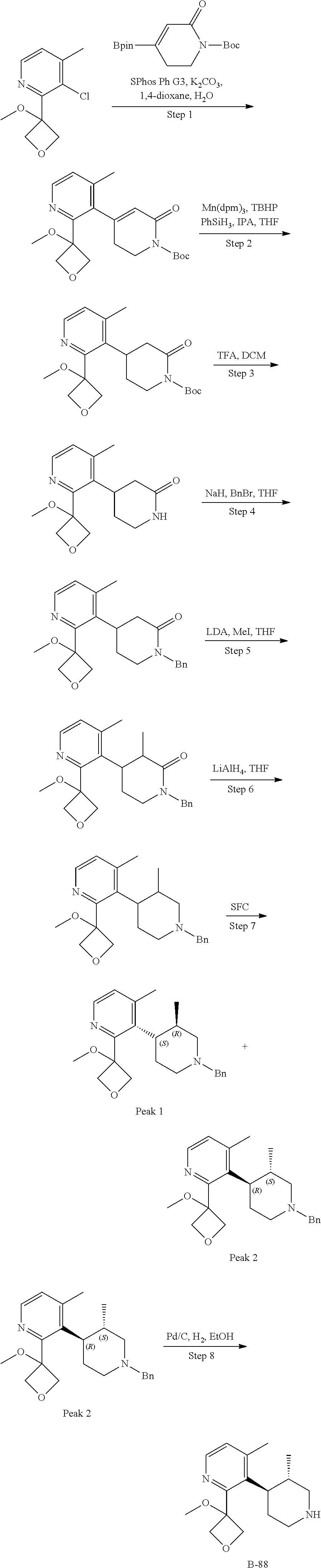

Step 1. To a vessel was added 3-chloro-2-(3-methoxyoxetan-3-yl)-4-methylpyridine (10 g, 46.8 mmol), tert-butyl 6-oxo-4-(4,4,5,5-tetramethyl-1,3,2-dioxaborolan-2-yl)-3,6-dihydropyridine-1(2H)-carboxylate (27.2 g, 84 mmol) and K₂CO, (22.64 g, 164 mmol) in 1,4-dioxane (115 mL) and H₂O (26 mL). The reaction mixture was purged with N₂ for 10 min. SPhos Pd G3 (1.826 g, 2.340 mmol) was added and purged with N₂ for 2 min. The reaction mixture was stirred at 110° C. for 16 h. The reaction mixture was cooled to rt, H₂O (200 mL) was added, and extracted with EtOAc (2×500 mL). The organic extract was dried over Na₂SO₄. The solution was filtered and concentrated in vacuo to give the crude material. The crude material was absorbed onto a plug of silica gel and purified by chromatography, eluting with a gradient of 60% to 70% EtOAc in hexanes to afford tert-butyl 2-(3-methoxyoxetan-3-yl)-4-methyl-6′-oxo-3′,6′-dihydro-[3,4′-bipyridine]-1′(2′H)-carboxylate.

Step 2. To a stirred solution of tert-butyl 2-(3-methoxyoctan-3-yl)-4-methyl-6′-oxo-3′,6′-dihydro-[3,4′-bipyridine]-1′(2′H)-carboxylate (9.5 g, 25.4 mmol) in IPA (190 mL) and THF (190 mL) was added Mn(dpm)₃, (7.67 g, 12.69 mmol) and phenylsilane (4.69 mL, 38.1 mmol). followed by TBHP (70% in H₂O) (6.95 mL, 50.7 mmol) at 0° C. under N₂ atmosphere. The reaction mixture was stirred at 0° C. for 30 min, then at 25° C. for 16 h. The reaction mixture was cooled and quenched with H₂O (300 mL) and extracted with EtOAc (3×200 mL). The organic layer was washed with H₂O (100 mL), dried over Na₂SO₄, filtered and concentrated under reduced pressure. The crude residue was purified by column chromatography (elution: 0-50% EtOAc in hexanes) to give tert-butyl 4-(2-(3-methoxyoxetan-3-yl)-4-methylpyridin-3-yl)-2-oxopiperidine-1-carboxylate. m/z (ESI): 377.2 (M+H)⁺. ¹H NMR (400 MHz, DMSO-d₆): δ (ppm) 8.32 (d, J=4.9 Hz, 1H), 7.24 (d, J=5.0 Hz, 1H), 5.10 (dd, J=42.0, 7.1 Hz, 2H), 4.78 (dd, J=16.6, 7.0 Hz, 2H), 3.87 (di. J=12.9, 4.6 Hz, 1H), 3.64-3.50 (m, 1H), 3.02 (dt, J=11.5, 6.1 Hz, 3H), 2.93 (s, 3H), 2.43 (s, 3H), 2.15 (t, J=10.8 Hz, 1H), 1.91 (dd, J=14.0, 5.0 Hz, 1H), 1.47 (s, 9H).

Step 3. To a solution of tert-butyl 4-(2-(3-methoxyoxetan-3-yl)-4-methylpyridin-3-yl)-2-oxopiperidine-1-carboxylate (5.5 g, 14.5 mmol) in DCM (40 mL) was added TFA (4.0 mL, 53.10 mmol) at 0° C. under N₂. The reaction mixture was stirred at rt for 2 h. The reaction mixture was quenched with saturated aqueous NaHCO₃ solution at 0° C., and extracted with 10% MeOH in DCM (4×60 mL). The reaction mixture was dried over Na₂SO₄, filtered and concentrated under reduced pressure to afford 4-(2-(3-methoxyoxetan-3-yl)-4-methylpyridin-3-yl)piperidin-2-one, which was used as is without further purification. m/z (ESI): 276.1 (M+H)⁺. ¹H NMR (400 MHz, DMSO-d₆): δ (ppm) 8.30 (dd, J=9.9, 4.9 Hz, 1H), 7.67 (d, J=3.9 Hz, 1H), 7.21 (dd, J=8.8, 4.9 Hz, 1H), 5.22 (d, J=7.0 Hz, 1H), 5.01 (d, J=7.0 Hz, 1H), 4.79 (dd, J=7.0, 0.9 Hz, 1H), 4.76-4.71 (m, 1H), 3.59 (s, 3H), 3.28-3.09 (m, 1H), 2.93 (s, 3H), 2.58 (t, J=13.0 Hz, 2H), 2.44 (d, J=9.5 Hz, 1H), 2.33-2.09 (m, 1H), 1.75 (q, J=8.2, 6.5 Hz, 2H).

Step 4: To a solution of 4-(2-(3-methoxyoxetan-3-yl)-4-methylpyridin-3-yl)piperidin-2-one (4.0 g, 14.48 mmol) in THF (60 mL) was added NaH (60% in mineral oil) (1.263 g, 29.0 mmol) at 0° C. under N₂. The reaction mixture was stirred at 0° C. for 30 min. Benzyl bromide (4.95 g, 29.0 mmol) was added next at 0° C., and stirred for 2 h. The reaction mixture was quenched with satd. aq. NH₄Cl solution (20 mL) and extracted with EtOAc (3×80 mL). The combined organic extracts were washed with H₂O (50 mL), dried over Na₂SO₄, filtered, and concentrated under reduced pressure. The crude residue was purified by column chromatography (elution: 0-60% EtOAc in hexanes) to give 1-benzyl-4-(2-(3-methoxyoxetan-3-yl)-4-methylpyridin-3-yl)piperidin-2-one. m/z (ESI): 367.0 (M+H)⁺. ¹H NMR (400 MHz, DMSO-d₆): δ (ppm) 8.32 (d, J=4.9 Hz, 1H), 7.42-7.34 (m, 2H), 7.32-7.18 (m, 4H), 5.22 (d, J=7.1 Hz, 1H), 5.03 (d, J=7.0 Hz, 1H), 4.73 (dd, J=7.1, 4.5 Hz, 2H), 4.66 (d, J=14.9 Hz, 1H), 4.49 (d, J=14.9 Hz, 11H), 3.30-3.21 (m, 2H), 2.92 (s, 4H), 2.79 (dd, J=17.3, 12.0 Hz, 1H), 2.48-2.39 (m, 4H), 2.25 (h, J=6.4 Hz, 1H), 1.81 (d, J=13.2 Hz, 1H).

Step 5. To a vessel was added 1-benzyl-4-(2-(3-methoxyoctan-3-yl)-4-methylpyridin-3-yl)piperidin-2-one (670 mg, 1.828 mmol) in THF (13.40 mL). The solution was cooled to −78° C. and LDA (2 M solution in THF, 1097 μL, 2.194 mmol) was added dropwise. After stirring for 30 min at −78° C. MeI (171 IL, 2.74 mmol) was added at −78° C., and the solution allowed to warm to rt and stirred for 2 h. The reaction was quenched with NH₄Cl while cooling in an ice bath. The extracts were separated. and the aqueous layer was extracted with EtOAc (2×100 mL). The combined organic extracts were dried over Na₂SO₄ and concentrated. The crude was purified by reverse phase chromatography using ACN in H₂O (35 to 40%) to give 1-benzyl-4-(2-(3-methoxyoxetan-3-yl)-4-methylpyridin-3-yl)-3-methylpiperidin-2-one. m/z (ESI): 381.3 (M+H)⁺. ¹H NMR (400 MHz, DMSO-d₆): δ (ppm) 8.32 (d, J=4.9 Hz, 1H), 7.37 (t, J=7.4 Hz, 2H), 7.31-7.17 (m, 4H), 5.16 (d, J=7.2 Hz, 1H), 5.04 (d, J=6.9 Hz, 1H), 4.72 (d, J=7.2 Hz, 1H), 4.68-4.46 (m, 3H), 3.02 (dd, J=11.5, 7.0 Hz, 2H), 2.93 (s, 3H), 2.71 (d, J=3.6 Hz, 2H), 2.46 (s, 3H), 2.24 (s, 1H), 1.85 (d, J=10.4 Hz, 1H), 0.91 (d, J=7.1 Hz, 3H).

Step 6. To a vessel was added 1-benzyl-4-(2-(3-methyloxetan-3-yl)-4-methylpyridin-3-yl)-3-methylpiperidin-2-one (530 mg, 1.393 mmol) in THF (32 mL). The reaction mixture was cooled to 0° C., and lithium aluminum hydride (2.0 M in THF) (1393 μL, 2.79 mmol) was added. The reaction mixture was heated and stirred at 60° C. for 15 min. The reaction mixture was cooled to 0° C., then sat Na₂SO₄ was added, and the mixture extracted with EtOAc (3×30 mL). The combined organic extracts were dried and the solvent was removed. the crude product was purified by reverse phase chromatography (ACN in H₂O 20 to 30%), to give 3-(1-benzyl-3-methylpiperidin-4-yl)-2-(3-methoxyoxetan-3-yl)-4-methylpyridine. m/z (ESI): 367.4 (M+H)⁺.

Step 7. The material was purified via SFC using a Chiralpak IG 250×20 mm, 5 μm column with a mobile phase of 1:1 ACN/MeOH in liquid CO₂ with a flow rate of 70 mL/min to pro, ide:

Peak 1: 3-((3R,4S,4R)-1-benzyl-3-methylpiperidin-4-yl)-2-(3-methoxyoxetan-3-yl)-4-methylpyridine ¹H NMR (400 MHz, DMSO-d₆): δ (ppm) 8.27 (d, J=4.8 Hz, 1H), 7.34-7.24 (m, 5H), 7.18 (d, J=5.0 Hz, 1H), 5.10 (dd, J=24.8, 7.2 Hz, 2H), 4.71 (dd, J=18.2, 7.1 Hz, 2H), 3.48 (s, 2H), 2.91 (s, 6H), 2.46 (s, 3H), 2.15-2.06 (m, 1H), 2.04-1.88 (m, 2H), 1.66-1.50 (m, 2H), 1.15 (dt, J=42.1, 7.2 Hz, 1H), 0.53 (d, J=6.7 Hz, 3H).
Peak 2: 3-((3S,4R)-1-benzyl-3-methylpiperidin-4-yl)-2-(3-methoxyoxetan-3-yl)-4-methylpyridine ¹H NMR (400 MHz, DMSO-d₆): δ (ppm) 8.27 (d, J=4.8 Hz, 1H), 7.48-7.20 (m, 5H), 7.18 (d, J=5.0 Hz, 1H), 5.18-4.99 (m, 2H), 4.84-4.59 (m, 2H), 3.48 (s, 2H), 2.91 (d, J=1.9 Hz, 6H), 2.46 (s, 4H), 2.12 (t, J=11.3 Hz, 1H), 1.95 (p, J=11.9, 11.1 Hz, 2H), 1.56 (dq, J=36.4, 12.3, 11.5 Hz, 2H), 1.24 (s, 1H), 0.53 (d, J=6.7 Hz, 3H).

Step 8. To a stirred solution of (peak 2) 3-((3S,4R)-1-benzyl-3-methylpiperidin-4-yl)-2-(3-methoxyoxetan-3-yl)-4-methylpyridine (160 mg, 0.437 mmol) in EtOH (1 mL) was added Pd—C (167 mg, 0.157 mmol) under nitrogen atmosphere and the reaction was continued to stir at 25° C. in a bladder under H₂ pressure for 1 h. The reaction mixture was filtered, and the filtrate was concentrated to get crude of 2-(3-methoxyoxetan-3-yl)-4-methyl-3-((3S,4R)-3-methylpiperidin-4-yl)pyridine. m/z (ESI): 277.0 (M+H)⁺.

¹H NMR (400 MHz, DMSO-d6): δ (ppm) 8.27 (d, J=4.9 Hz, 1H), 7.17 (d, J=4.8 Hz, 1H), 5.17-5.03 (m, 2H), 4.81-4.66 (m, 2H), 4.10 (d, J=5.4 Hz, 11H), 3.17 (d, J=4.5 Hz, 2H), 3.02 (t, J=10.2 Hz, 2H), 2.92 (d, J=2.6 Hz, 3H), 2.47 (s, 3H), 2.26-1.86 (m, 4H), 0.56 (dd, J=18.3, 5.7 Hz, 3H).

The intermediate in the table below as prepared in a fashion similar to that described above. Synthesis of the building blocks is either described above or from commercial sources.

| | | | m/z (ESI): |
|---|---|---|---|
| Int. # | Chemical Structure | Name | (M + H)⁺ |
| B-110 | 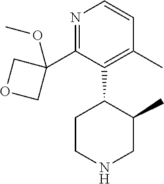 [SMILES: COC1(c2nccc(C)c2[C@H]2CCNC[C@@H]2C)COC1] | 2-(3-methoxyoxetan-3-yl)-4-methyl-3-((3R,4S)- 3-methylpiperidin-4-yl)pyridine | 277.0 |

### Benzyl 4-(3-(azetidin-1-yl)-5-methylpyridazin-4-yl)piperidine-1-carboxylate (Intermediate B-89)

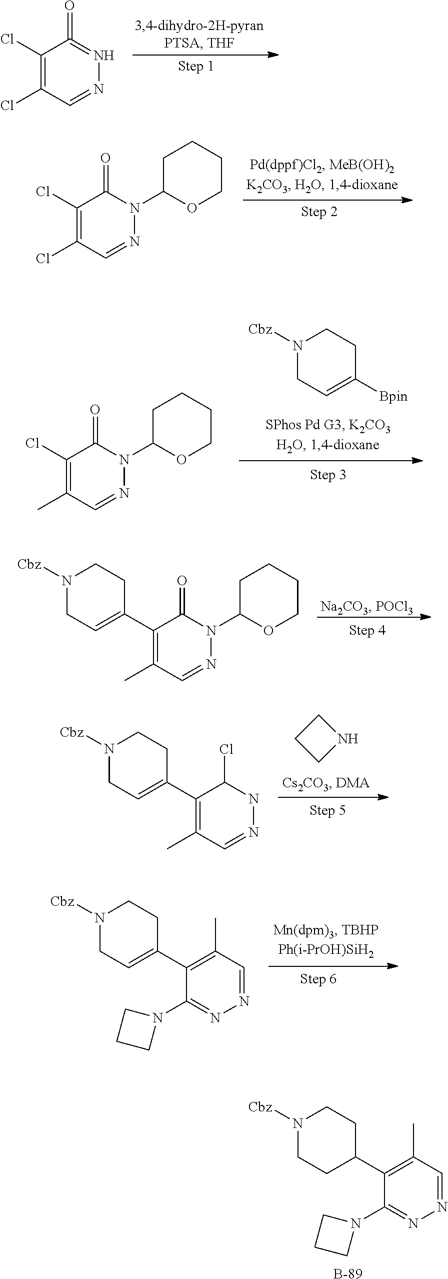

Step 1. A mixture of 4-methylbenzenesulfonic acid hydrate (1.107 g, 5.82 mmol, Sigma-Aldrich. Inc.), 4,5-dichloropyridazin-3(2H)-one (12 g, 72.7 mmol, Combi-Blocks, Inc.), and 3,4-dihydro-2H-pyran (8.63 mL, 95 mmol, Oakwood Products. Inc.) in THF (100 mL) was stirred at reflux for 18 h. An additional aliquot of 3,4-dihydro-2H-pyran (5 mL) was added at 16 h and then the reaction mixture was removed from heat and allowed to stir at rt for 48 h. The mixture was concentrated in vacuo. dissolved in EtOAc (150 mL) and washed with 2N NaOH (100 mL). The organic layer was dried over MgSO₄, filtered, and concentrated in vacuo to afford the crude product which was chromatographed, eluting with a gradient of 0% to 50% EtOAc in heptane, to provide 4,5-dichloro-2-(tetrahydro-2H-pyran-2-yl)pyridazin-3(2H)-one. The compound was purified to yield 4,5-dichloro-2-(tetrahydro-2H-pyran-2-yl)pyridazin-3(2H)-one). m/z (ESI): 164.9 (M−THP+H)⁺. ¹H NMR (401 MHz, DMSO-d₆): δ ppm 8.24 (s, 1H), 5.85 (dd, J=10.4, 2.1 Hz, 1H), 3.93-4.01 (m, 1H), 3.62-3.67 (m, 1H), 1.99-2.10 (m, 1H), 1.91-1.97 (n, 1H), 1.63-1.74 (m, 2H), 1.46-1.57 (m, 2H)

Step 2. To a vessel was added a mixture of Pd(dppf)Cl₂ DCM (0.393 g, 0.482 mmol, Strem Chemicals, Inc.), methylboronic acid (0.529 g, 8.83 mmol, Oakwood Products, Inc.), 4,5-dichloro-2-(tetrahydro-2H-pyran-2-yl)pyridazin-3(2H)-one (2 g, 8.03 mmol) and potassium carbonate, anhydrous (2.77 g, 20.07 mmol, Sigma-Aldrich, Inc.), and the reaction vessel was purged with N₂. Then the reaction mixture was dissolved in 1,4-dioxane/H₂O (20/2 mL) and heated at 95° C. for 17 h. The reaction mixture was allowed to cool down to rt. diluted with H₂O and extracted with EtOAc. The combined organic extracts were dried over Na₂SO₄, filtered, concentrated and chromatographed on silica gel using 0-70% EtOAc in heptane to afford 5-chloro-4-methyl-2-(tetrahydro-2H-pyran-2-yl)pyridazin-3(2H)-one. m/z (ESI): 229.1 (M+H)⁺

Step 3. To a vial was added K₂CO. (907 mg, 6.56 mmol, Sigma-Aldrich, Inc.), SPhos Pd G3 (227 mg, 0.262 mmol, Sigma-Aldrich, Inc.). N-Cbz-1,2,3,6-tetrahydropyridine-4-boronic acid (1081 mg, 3.15 mmol, Combi-Blocks, Inc.), and 4-chloro-5-methyl-2-(tetrahydro-2H-pyran-2-yl)pyridazin-3(2H)-one (600 mg, 2.62 mmol), 1,4-dioxane (6 mL) and H₂O (0.6 mL). The reaction mixture was bubbled with nitrogen flow for 15 min before heating at 95° C. for 2 h. The reaction was quenched by sat. aq. NH₄Cl (5 mL). The crude material was extracted using EtOAc (3×). washed with brine, dried through Na₂SO₄, and concentrated. The crude material was purified by chromatography, eluting with a gradient of 5% to 50% acetone in heptane to provide benzyl 4-(5-methyl-3-oxo-2-(tetrahydro-2H-pyran-2-yl)-2,3-dihydropyridazin-4-yl)-3,6-dihydropyridine-1(2H)-carboxylate. m/z (ESI): 410.0 (M+H)⁺.

Step 4. To a glass vial was added phosphorus oxide trichloride (910 μL, 9.77 mmol, Sigma-Aldrich, Inc.) and benzyl 4-(5-methyl-3-oxo-2-(tetrahydro-2H-pyran-2-yl)-2,3-dihydrophenazin-4-yl)-3,6-dihydropyridine-1(2H)-carboxylate (800 mg, 1.954 mmol). The reaction mixture was heated to 80° C. for 1 h, then quenched by 10 wt % Na₂CO₃ aq solution (10 mL). The reaction mixture was extracted by DCM (3×) and the combined organic extracts were dried over Na₂SO₄, and concentrated. The crude material was purified by chromatography, eluting with a gradient of 5% to 30% acetone in heptane to afford benzyl 4-(3-chloro-5-methylpyridazin-4-yl)-3,6-dihydropyridine-1(2H)-carboxylate.

Step 5. To a glass vial was added benzyl 4-(3-chloro-5-methylpyridazin-4-yl)-3,6-dihydropyridine-1(2H)-carboxylate (300 mg, 0.873 mmol) and Cs₂CO₃ (853 mg, 2.62 mmol, Sigma-Aldrich. Inc.) in DMA (5 mL). Then 1,3-propyleneimine (0.100 mL, 1.745 mmol, Combi-Blocks. Inc.) was added, and the reaction mixture was heated to 100° C. for 18 h. The mixture was brought to rt, diluted with EtOAc and washed with H₂O. The organic extract was dried over Na₂SO₄, filtered, and the solvent removed under vacuum. The crude material was purified by chromatography, eluting with a gradient of 0% to 100% acetone in heptane to provide benzyl 4-(3-(azetidin-1-yl)-5-methylpyridazin-4-yl)-3,6-dihydropyridine-1(2H)-carboxylate. m/z (ESI): 365.2 (M+H)⁺.

Step 6. To a degassed vessel was added a solution of benzyl 4-(3-(azetidin-1-yl)-5-methylpyridazin-4-yl)-3,6-dihydropyridine-1(2H)-carboxylate (180 mg, 0.494 mmol) and tris(2,2,6,6-tetramethyl-3,5-heptanedionato)manganese(III) (59.7 mg, 0.099 mmol, Strem Chemicals, Inc.) in IPA (10 mL) at 0° C., then tert-butyl hydrogen peroxide (0.296 mL, 1.482 mmol, Sigma-Aldrich, Inc.) and phenylsilane (0.160 mL, 1.482 mmol. Chem Impex) were added dropwise and the reaction mixture was stirred at 0° C. and allowed to warm to it for 18 h. The reaction mixture was quenched by the addition of NH₄OH, H₂O and EtOAc. The organic phase was separated, and the aqueous phase was extracted with EtOAc. The combined organic extracts were washed with brine, dried over Na₂SO₄, filtered, concentrated, and chromatographed on silica gel using 0-1(00% EtOAc in heptane to afford benzyl 4-(3-(azetidin-1-yl)-5-methylpyridazin-4-yl)piperidine-1-carboxylate. m/z (ESI): 367.3 (M+H)⁺

The intermediates in the table below were prepared in a fashion similar to that described above. Synthesis of the building blocks is either described above or from commercial sources.

| | | | m/z (ESI): |
|---|---|---|---|
| Int. # | Chemical Structure | Name | (M + H)⁺ |
| B-90 | 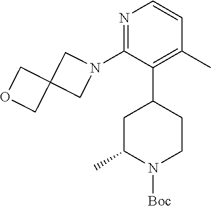 [SMILES: Cc1ccnc(N2CC3(COC3)C2)c1C1CCN(C(=O)OC(C)(C)C)[C@H](C)C1] | tert-butyl (2R)-2-methyl-4-(4-methyl-2- (2-oxa-6-azaspiro[3.3]heptan-6-yl)pyridin-3- yl)piperidine-1-carboxylate | 388.3 |
| B-91 | 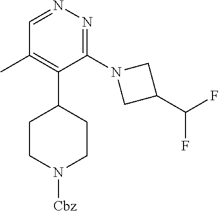 [SMILES: Cc1cnnc(N2CC(C(F)F)C2)c1C1CCN(C(=O)OCc2ccccc2)CC1] | benzyl 4-(3-(3-(difluoromethyl)azetidin-1-yl)-5- methylpyridazin-4-yl)piperidine-1-carboxylate | 417.2 |
| B-92 | 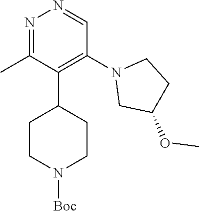 | tert-butyl (S)-4-(5-(3-methoxypyrrolidin-1-yl)-3- methylpyridazin-4-yl)piperidine-1-carboxylate | 377.3 |
| B-93 | 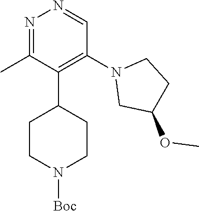 | tert-butyl (R)-4-(5-(3-methoxypyrrolidin-1-yl)-3- methylpyridazin-4-yl)piperidine-1-carboxylate | 377.3 |
| B-94 | 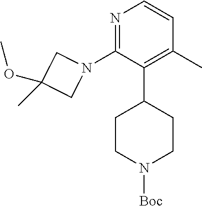 | tert-butyl 4-(2-(3-methoxy-3-methylazetidin-1-yl)- 4-methylpyridin-3-yl)piperidine-1-carboxylate | 376.3 |
| B-95 | 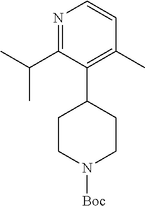 [SMILES: Cc1ccnc(C(C)C)c1C1CCN(C(=O)OC(C)(C)C)CC1] | tert-butyl 4-(2-isopropyl-4-methylpyridin-3- yl)piperidine-1-carboxylate | 319.2 |
| B-96 | 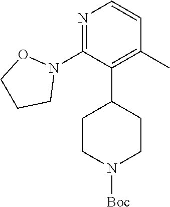 [SMILES: Cc1ccnc(N2CCCO2)c1C1CCN(C(=O)OC(C)(C)C)CC1] | tert-butyl 4-(2-(isoxazolidin-2-yl)-4-methylpyridin- 3-yl)piperidine-1-carboxylate | 348.2 |
| B-97 | 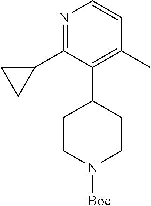 [SMILES: Cc1ccnc(C2CC2)c1C1CCN(C(=O)OC(C)(C)C)CC1] | tert-butyl 4-(2-cyclopropyl-4-methylpyridin-3- yl)piperidine-1-carboxylate | 317.2 |

### 4-(1-Methyl-4-(trifluromethyl)-1H-imidazol-5-yl)piperidine (Intermediate B-98)

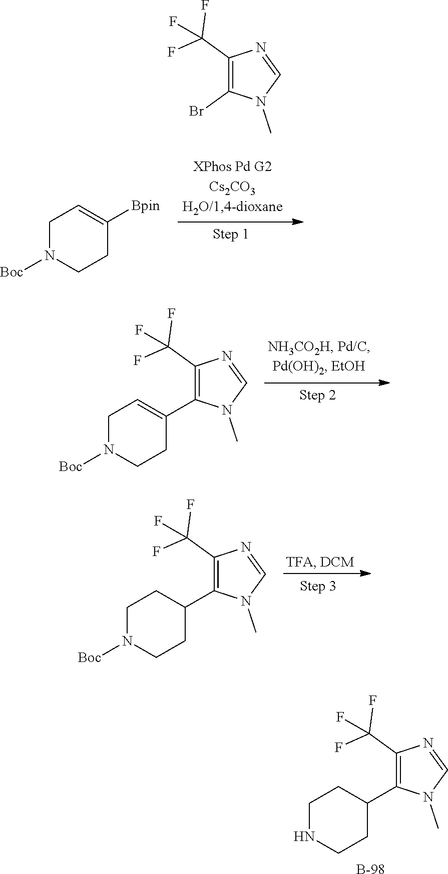

Step 1. To a vessel was added XPhos Pd G2 (271 mg, 0.344 mmol, Ambeed. Inc.) and 5-bromo-1-methyl-4-(trifluoromethyl)-1H-imidazole (526 mg, 2.296 mmol), Cs₂CO₃ (748 mg, 2.2% mmol, Combi-Blocks Inc.), tert-butyl 4-(4,4,5,5-tetramethyl-1,3,2-dioxaborolan-2-yl)-3,6-dihydropyridine-1(2H)-carboxylate (710 mg, 2.296 mmol), 1,4-dioxane (5 mL) and H₂O (0.5 mL). The reaction mixture was heated to 85° C., and stirred for 75 min. The mixture was cooled to rt and the catalyst was filtered using celite. The reaction was then cooled and extracted with DCM (5% MeOH) 3×10 mL. The combined organics were washed with brine, dried over MgSO₄, filtered, and concentrated in vacuo. The crude material was absorbed onto a plug of silica gel and purified by chromatography, eluting with a gradient of 10% to 70% EtOAc/EtOH 3:1 in heptane+2% TEA to provide tert-butyl 4-(1-methyl-4-(trifluoromethyl)-1H-imidazol-5-yl)-3,6-dihydropyridine-1(2H)-carboxylate. m/z (ESI): 332.2 (M+H)⁺.

Step 2. To a vessel was added tert-butyl 4-(1-methyl-4-(trifluoromethyl)-1H-imidazol-5-yl)-3,6-dihydropyridine-1(2H)-carboxylate (555 mg, 1.675 mmol) in EtOH (3350 μL) followed by the addition of ammonium formate (2112 mg, 33.5 mmol, Sigma-Aldrich, Inc.), palladium hydroxide carbon (706 mg, 1.005 mmol, Combi-Blocks, Inc.) and palladium on acti ated carbon (1070 mg, 1.005 mmol, Sigma-Aldrich, Inc.). The reaction was heated to 65° C. for 18 h. The crude mixture was filtered using celite and the filtrate was concentrated under reduced pressure to afford tert-butyl 4-(1-methyl-4-(trifluoromethyl)-1H-imidazol-5-yl)piperidine-1-carboxylate which was used without purification in the next step. m/z (ESI) 334.2 (M+H)⁺.

Step 3. To a vessel was added TFA (949 mg, 949 μL, 8.32 mmol. Apollo Scientific Ltd.) and tert-butyl 4-(1-methyl-4-(trifluoromethyl)-1H-imidazol-5-yl)piperidine-1-carboxylate (555 mg, 1.665 mmol) in DCM (3330 μL). The mixture was stirred for 1 h. The reaction mixture was concentrated under vacuo to afford 4-(1-methyl-4-(trifluoromethyl)-1Himidazol-5-yl)piperidine and used as is in the next step. m/z (ESI) 234.1 (M+H)⁺.

The intermediates in the table below were prepared in a fashion similar to that described above. Synthesis of the building blocks is either described above or from commercial sources.

| | | | m/z (ESI): |
|---|---|---|---|
| Int. # | Chemical Structure | Name | (M + H)⁺ |
| B-99 | 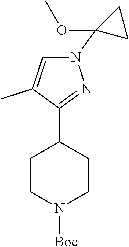 [SMILES: COC1(n2cc(C)c(C3CCN(C(=O)OC(C)(C)C)CC3)n2)CC1] | tert-butyl 4-(1-(1-methoxycyclopropyl)-4-methyl- 1H-pyrazol-3-yl)piperidine-1-carboxylate | 336.2 |
| B-100 | 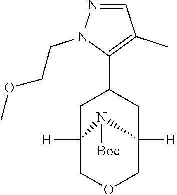 [SMILES: COCCn1ncc(C)c1C1C[C@H]2COC[C@@H](C1)N2C(=O)OC(C)(C)C] | tert-butyl (1R,5S)-7-(1-(2-methoxyethyl)-4-methyl- 1H-pyrazol-5-yl)-3-oxa-9-azabicyclo[3.3.1]nonane- 9-carboxylate | 366.1 |
| B-101 | 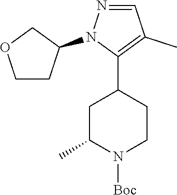 [SMILES: Cc1cnn([C@H]2CCOC2)c1C1CCN(C(=O)OC(C)(C)C)[C@H](C)C1] | tert-butyl (2R)-2-methyl-4-(4-methyl-1-((S)- tetrahydrofuran-3-yl)-1H-pyrazol-5-yl)piperidine- 1-carboxylate | 350.2 |
| B-102 | 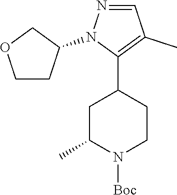 [SMILES: Cc1cnn([C@@H]2CCOC2)c1C1CCN(C(=O)OC(C)(C)C)[C@H](C)C1] | tert-butyl (2R)-2-methyl-4-(4-methyl-1-((R)- tetrahydrofuran-3-yl)-1H-pyrazol-5-yl)piperidine- 1-carboxylate | 350.2 |
| B-103 | 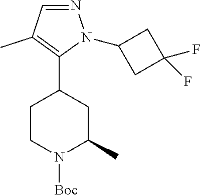 [SMILES: Cc1cnn(C2CC(F)(F)C2)c1C1CCN(C(=O)OC(C)(C)C)[C@H](C)C1] | tert-butyl (2R)-4-(1-(3,3-difluorocyclobutyl)-4- methyl-1H-pyrazol-5-yl)-2-methylpiperidine-1- carboxylate | 370.2 |

### 4-(1-Cyclopropyl-4-methyl-1H-pyrazol-5-yl)-3,3-dimethylpiperidine (Intermediate B-105)

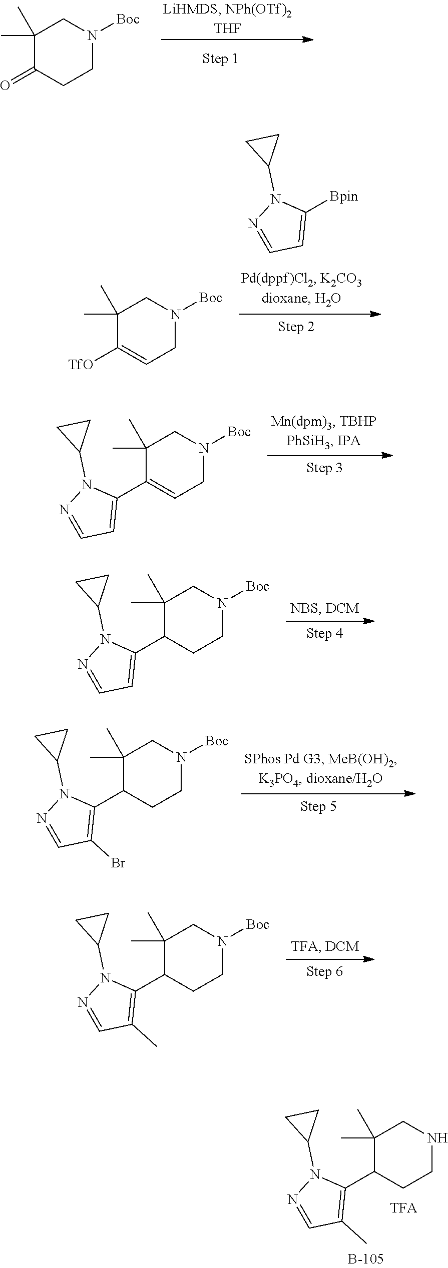

Step 1. To a solution of LiHMDS (1 M in THF, 121 mL, 121 mmol) was added tert-butyl 3,3-dimethyl-4-oxopiperidine-1-carboxylate (25 g, 110 mmol) in THF (100 mL) at −60° C. Then the mixture was stirred at −60° C. for 1.5 h. A solution of 1,1,1-trifluoro-N-phenyl-N-((trifluoromethyl)sulfonyl) methanesulfonamide (43.2 g, 121 mmol) in THF (100 mL) was added dropwise at −60° C., and stirred at 0° C. for 2 h. The reaction was quenched by sat. aq. NH₄Cl (500 mL), dried over Na₂SO₄, filtered and the filtrate was concentrated under reduced pressure. The residue was purified by column chromatography (SiO₂, pet ether/EtOAc=1:0 to 10:1) to give tert-butyl 3,3-dimethyl-4-(((trifluoromethyl)sulfonyl)oxy)-3,6-dihydropyridine-1(2H)-carboxylate.

Step 2. A mixture of 1-cyclopropyl-5-(4,4,5,5-tetramethyl-1,3,2-dioxaborolan-2-yl)-1H-pyrazole (15 g, 64.1 mmol), tert-butyl 3,3-dimethyl-4-((trifluoromethyl)sulfonyl)oxy)-3,6-dihydropyridine-1(2H)-carboxylate (34.5 g, 96 mmol). Pd (dppf)Cl₂ (4.69 g, 6.41 mmol) and K₂CO₃ (26.6 g, 192 mmol) in 1,4-dioxane (200 mL) and H₂O (10 mL) was degassed and purged with N₂ (3×), and then the mixture was stirred at 90° C. for 12 h under N₂ atmosphere. The reaction mixture was filtered, and the filtrate was concentrated under reduced pressure. The residue was first purified by column chromatography (SiO₂. pet ether/EtOAc=10/0 to 1/1) and then by reversed-phase MPLC (column. DAC-150 luna; mobile phase: H₂O; ACN 20%-40%, 40 min) to give tert-butyl 4-(1-cyclopropyl-1H-pyrazol-5-yl)-3,3-dimethyl-3,6-dihydropyridine-1(2H)-carboxylate. m/z (ESI): 318.2 (M+H)⁺.

Step 3. To a vial was added tris(((Z)-2,2,6,6-tetramethyl-5-oxohept-3-en-3-yl)oxy)manganese (2.48 g, 4.10 mmol), tert-butyl 4-(1-cyclopropyl-1H-pyrazol-5-yl)-3,3-dimethyl-3,6-dihydroxyindane-1(2H-carboxylate (13 g, 41.0 mmol). phenylsilane (13.3 g, 123 mmol), and IPA (200 mL). The reaction mixture was degassed for 30 min. cooled to 0° C., and hydroperoxy-2-methylpropane (18.62 mL, 102 mmol) was added. The reaction mixture was warmed to 20° C., and stirred 12 h. The reaction was quenched by the addition of 10% wt NH₃·H₂O (400 mL). The crude material was extracted with EtOAc (300 mL×3). The combined organic extracts were washed with brine (100 mL), dried through Na₂SO₄, filtered, and concentrated. The residue was purified by column chromatography (SiO₂, pet ether/EtOAc=20/1 to 1/1) to give tert-butyl 4-(1-cyclopropyl-1H-pyrazol-5-yl)-3,3-dimethylpiperidine-1-carboxylate. m/z (ESI): 320.3 (M+H)⁺.

Step 4. To a solution of tert-butyl 4-(1-cyclopropyl-1H-pyrazol-5-yl)-3,3-dimethylpiperidine-1-carboxylate (3 g, 9.39 mmol) in DCM (30 mL) was added NBS (2.507 g, 14.09 mmol). Then the mixture was stirred at 20° C. for 8 h. The reaction mixture was concentrated under reduced pressure and the residue was purified by column chromatography (SiO₂, pet ether/EtOAc=20:1 to 1:1) to give tert-butyl 4-(4-bromo-1-cyclopropyl-1H-pyrazol-5-yl)-3,3-dimethylpiperidine-1-carboxylate. m/z (ESI): 400.1 (M+H)⁺.

Step 5. A mixture of tert-butyl 4-(4-bromo-1-cyclopropyl-1H-pyrazol-5-yl)-3,3-dimethylpiperidine-1-carboxylate (1.3 g, 3.26 mmol), MeB(OH)₂ (0.391 g, 6.53 mmol), SPhos Pd G3 (0.255 g, 0.326 mmol) and K₃PO₄ (0.875 g, 6.53 mmol) in 1,4-dioxane (10 mL) and H₂O (1 mL) was degassed and purged with N₂ (3×), and then the mixture was stirred at 100° C. for 12 h under N₂ atmosphere. The reaction mixture was concentrated under reduced pressure to remove solvent. The residue was purified by column chromatography (SiO₂, pet ether/EtOAc=10:1 to 0:1) followed by prep-HPLC (column, Waters Xbridge BEH C18 100*30 mm*10 um; mobile phase: [H₂O(NH₄HCO₃)]; CAN %: 40%-60%, 8 min) to give tert-butyl 4-(1-cyclopropyl-4-methyl-1H-pyrazol-5-yl)-3,3-dimethylpiperidine-1-carboxylate. m/z (ESI): 334.2 (M+H)⁺.

Step 6. To a solution of tert-butyl 4-(1-cyclopropyl-4-methyl-1H-pyrazol-5-yl)-3,3-dimethylpiperidine-1-carboxylate (500 mg, 1.50 mmol) in DCM (3 mL) was added TFA (1 mL). Then the mixture was stirred at 20° C. for 12 h. The reaction mixture was concentrated under reduced pressure to give 4-(1-cyclopropyl-4-methyl-1H-pyrazol-5-yl)-3,3-dimethylpiperidine 2,2,2-trifluoroacetate (600 mg, crude) that was used in the next step without purification. m-r (ESI): 234.1 (M+H)⁺.

### 2-(3-Methoxyoxetan-3-yl)-4-methyl-3-(piperidin-4-yl)pyridine (Intermediate B-106)

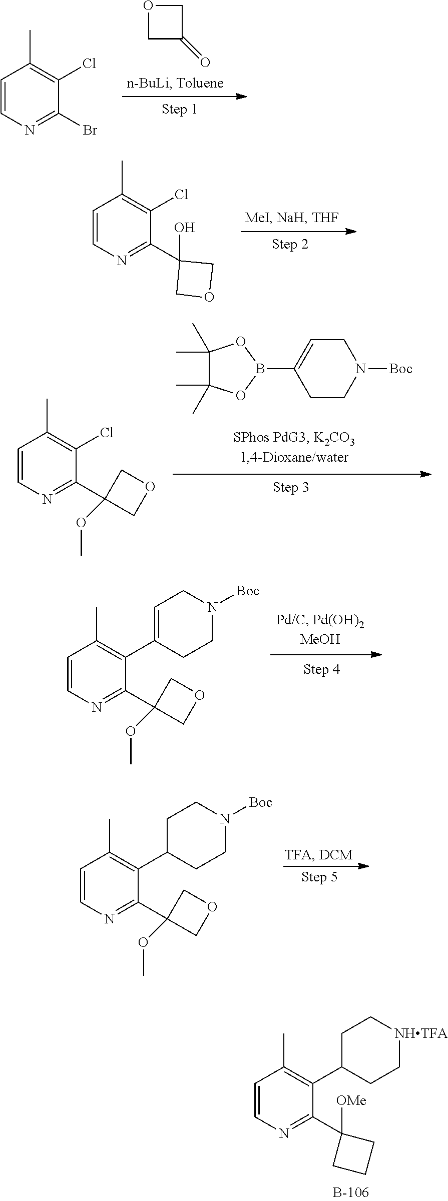

Step 1. To a solution of 2-bromo-3-chloro-4-methylpyridine (600 g, 2906 mmol) in toluene (9 L) at −78° C. was added n-BuLi (2.5 M solution in hexane) (1162 mL, 2906 mmol) dropwise for 60 min and the reaction mixture was stirred for 1 h at −78° C. A solution of oxetan-3-one (188 g, 2615 mmol) in toluene (300 mL) was added over 30 min and stirred for 1 h at −78° C. The reaction mixture was slowly quenched with said. NH₄Cl (10 L), extracted with EtOAc (3×4 L), and subjected to workup to give 3-(3-chloro-4-methylpyridin-2-yl)oxetan-3-ol. m/z (ESI): 200.1 (M+H)⁺. ¹H NMR (400 MHz, DMSO-d₆): δ 8.36 (d, J=4.8 Hz, 1H), 7.40 (dd, J=4.9, 0.9 Hz, 1H), 6.46 (s, 1H), 5.15 (dd, J=6.9, 1.0 Hz, 2H), 4.69 (dd, J=6.9, 1.0 Hz, 2H), 2.38 (d, J=0.7 Hz, 3H).

Step 2. To a stirred solution of 3-(3-chloro-4-methylpyridin-2-yl)oxetan-3-ol (260 g, 1302 mmol) in THF (2500 mL) at 0° C. was added NaH (104 g, 2605 mmol, 60% in oil) portion wise for 15 min and the reaction mixture was stirred at 0° C. for 30 min. Methyl iodide (163 mL, 2605 mmol) was added for 20 min and the solution was slowly warmed to rt and stirred for 16 h. The reaction mixture was quenched with ice water (10 L) and extracted with EtOAc (2×5 L). The combined organic extracts were dried over Na₂SO₄ and concentrated to give 3-chloro-2-(3-methoxyoxetan-3-yl)-4-methylpyridine. m/z (ESI): 214.3 (M+H)⁺. ¹H NMR (400 MHz, DMSO-d₆): δ 8.42 (d, J=4.9 Hz, 1H), 7.46 (dd, J=4.9, 0.8 Hz, 1H), 5.08 (dd, J=7.3, 1.1 Hz, 2H), 4.76 (dd, J=7.4, 1.1 Hz, 2H), 2.97 (s, 3H), 2.40 (d, J=0.7 Hz, 3H).

Step 3. A stirred solution of 3-chloro-2-(3-methoxyoxetan-3-yl)-4-methylpyridine (344 g, 1610 mmol), tert-butyl 4-(4,4,5,5-tetramethyl-1,3,2-dioxaborolan-2-yl)-3,6-dihydropyridine-1(2H)-carboxylate (597 g, 1932 mmol), K₂CO₃ (668 g, 4830 mmol) in 1,4-dioxane (3440 mL), H₂O (1032 mL) was degassed for 5 min and SPhos Pd G3 (62.8 g, 81 mmol) was added. The reaction mixture was heated to 100° C. for 16 h and concentrated under reduced pressure. The crude obtained was suspended in water (5 L) and extracted with EtOAc (3×5 L). The combined organic extracts were dried over Na₂SO₄, filtered. and concentrated under vacuum. The crude material was absorbed onto a plug of silica gel and purified by chromatography eluting with a gradient of 25% to 50% EtOAc in hexanes, to give tert-butyl 2-(3-methoxyoxetan-3-yl)-4-methyl-3′,6-dihydro-[3,4′-bipyridine]-1′(2′H)-carboxylate. m/z (ESI): 361.3 (M+H)⁺. ¹H NMR (401 MHz, DMSO-d₆): δ 8.35 (d, J=4.9 Hz, 1H), 7.28 (dd, J=4.9, 0.8 Hz, 1H), 5.49 (s, 1H), 5.25 (d, J=7.2 Hz, 1H), 4.86 (d, J=7.3 Hz, 1H), 4.39-4.63 (m, 2H), 3.92 (d, J=5.2 Hz, 2H), 3.51 (t, J=5.3 Hz, 2H), 2.94 (s, 3H), 2.43-2.01 (m, 5H), 1.44 (s, 9H).

Step 4. To a stirred solution of tert-butyl 2-(3-methoxyoxetan-3-yl)-4-methyl-3′,6′-dihydro-[3,4′-bipyridine]-1′(2′H)-carboxylate (60 g, 166 mmol) in MeOH (1000 mL) at rt. was added 10% Pd/C (50% wet) (30 g, 28.2 mmol) and 20% palladium hydroxide on carbon (30 g, 214 mmol). The reaction mixture was degassed thoroughly and stirred under a balloon of H₂ (10 psi) for 5 d at rt. The reaction mixture was filtered through celite pad and washed with 50% DCM in MeOH (2000 mL). The filtrate was concentrated under reduced pressure and purified via SFC using a ChiralPak 10 (250×50) mm, 5 μm, column with a mobile phase of liquid CO₂: [0.2% NH₃ in ACN:EtOH (2:8)] using a flowrate of 150 mL/min to give tert-butyl 4-(2-(3-methoxyoxetan-3-yl)-4-methylpyridin-3-yl)piperidine-1-carboxylate. m/z (ESI): 363.1 (M+H)⁺. ¹H NMR (400 MHz, DMSO-d₆): δ 8.28 (d, J=4.9 Hz, 1H), 7.18 (d, J=5.0 Hz, 1H), 5.11 (d, J=7.1 Hz, 2H), 4.79 (dd, J=7.0, 1.0 Hz, 2H), 4.05 (d, J=12.9 Hz, 1H), 3.32 (s, 2H), 2.93 (s, 2H), 2.55-2.80 (m, 2H), 2.42 (s, 2H), 1.93 (qd, J=12.6, 4.3 Hz, 2H), 1.50 (d, J=12.8 Hz, 2H), 1.42 (s, 11H).

Step 5. To a stirred solution of tert-butyl 4-(2-(3-methoxyoxetan-3-yl)-4-methylpyridin-3-yl)piperidine-1-carboxylate (85 g, 235 mmol) in DCM (850 mL) at 0° C. was added TFA (250 mL, 3245 mmol) dropwise for 15 min and the reaction mixture was stirred at rt for 3 h. The reaction mixture was concentrated under reduced pressure and the crude was co-evaporation with toluene (3×150 mL) followed by trituration with diethyl ether (4×150 mL) and concentrated under reduced pressure to give 2-(3-methoxyoxetan-3-yl)-4-methyl-3-(piperidin-4-yl)pyridine as TFA salt. m/z (ESI): 263.2 (M+H)⁺.

### 2-Methyl-3-(piperidin-4-yl)-5,6-dihydro-4H-pyrrolo[1,2-b]pyrazole (Intermediate B-107)

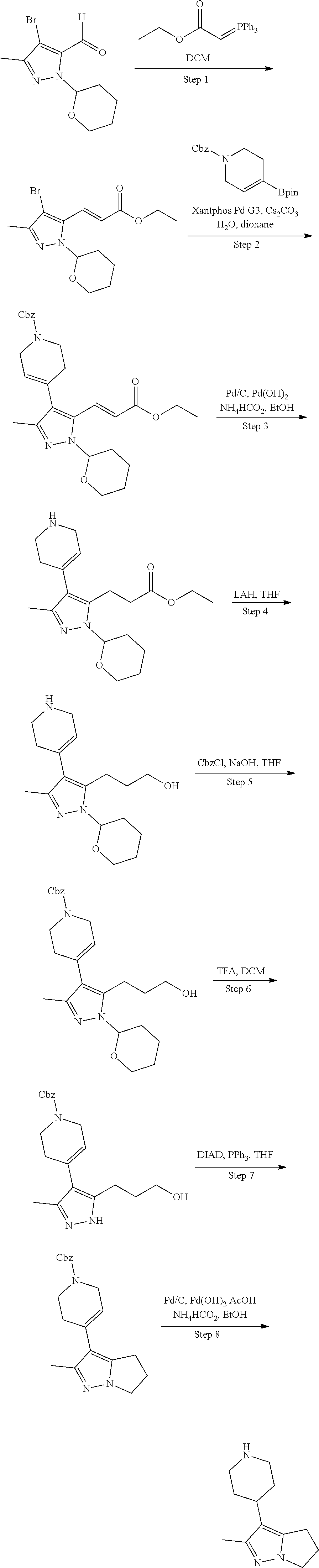

Step 2. To a 150-mL round-bottomed flask was added 4-bromo-5-methyl-2-(oxan-2-yl)pyrazole-3-carbaldehyde (2 g, 7.32 mmol, Combi-Blocks, Inc.) and ethyl 2-(triphenylphosphoranylidene)acetate (2.81 g, 8.05 mmol, Ambeed, Inc.) in dichloromethane (36.6 mL), and the reaction was left stirring at to. After 7.5 h, the reaction mixture was diluted with NaHCO₃ (20 mL), extracted with EtOAc (3×15 mL), dried over MgSO₄, filtered and concentrated. The crude material was absorbed onto a plug of silica gel and purified by chromatography, eluting with a gradient of 0% to 100% 3:1 EtOAc:EtOH in heptane, to provide ethyl (E)-3-(4-bromo-3-methyl-1-(tetrahydro-2H-pyran-2-yl)-1H-pyrazol-5-yl)acrylate. m/z (ESI): 4.9, 366.9 (M+Na)+.

Step 2. To a vial was added ethyl (E)-3-(4-bromo-3-ethyl-1-tetrahedro-2H-pyran-2-yl)-1H-pyrazol-5-2yl)acrylate (800 mg, 2.331 mmol) and N-Cbz-1,2,3,6-(tetrahydropyrine-4-boronic acid (880 mg, 2.56 mmol, Combi-Blocks. Inc.) in 1,4-dioxane (10.6 mL) and H₂O (1.1 mL). To the mixture was then added Cs₂CO₃ (2278 mg, 699 mmol) followed by Xantphos Pd G3 (241 mg, 0.233 mmol. Strem Chemicals. Inc.) and the reaction was heated to 100° C. for 18 h. The reaction was diluted with sat. NaHCO₃ (15 mL), extracted with EtOAc (3×15 mL), dried over MgSO₄, filtered and concentrated. The crude material was absorbed onto a plug of silica gel and purified by chromatography, eluting with a gradient of 0% to 100% EtOAc in heptane, to provide benzyl (E)-4-(5-(3-ethoxy-3-oxoprop-1-en-1-yl)-3-methyl-1-(tetrahydro-2H-pyran2Hpyran-2-yl)-1H-pyrazol-4-yl)-3,6-dihydropyridine-1(2H)-carboxylate m/z (ESI): 502.2 (M+Na)⁺.

Step 3. To a vial was added benzyl (E)-4-(5-(3-ethoxy-3-oxoprop-1-en-1-yl)-3-methyl-1-(tetrahydro-2H-pyran-2-yl)-1H-pyrazol-4-yl)-3,6-dihydropyridone-1(2H)-carboxylate (1.120 g, 2.335 mmol) and ammonium formate (1.473 g, 23.35 mmol, Sigma-Aldrich, Inc.) in EtOH (7.78 mL). To the mixture was added palladium on activated carbon (0.075 g, 0.701 mmol, Sigma-Aldrich. Inc.) and palladium hydroxide on carbon (0.098 g, 0.701 mmol, Combi-Blocks. Inc.) and the reaction was heated to 50° C. for 18 h. The temperature was increased to 70° C., and stirred for 4.5 h. The reaction was filtered through a pad of celite and concentrated to give ethyl 3-(3-methyl-1-(tetrahydro-2H-pyran-2-yl)-4-(1,2,3,6-tetrahydropyridin-4-yl-1H-pyrazol-5-yl). m/z (ESI): 348.3 (M+H)⁺.

Step 4. To a glass vial was added ethyl 3-(3-methyl-1-(tetrahydro-2H-pyran-2-yl)-4-(1,2,3,6-tetrahydropyridin-4-yl)-1H-pyrazol-5-yl)propanoate (860 mg, 2.475 mmol) in THF (25 mL) The solution was cooled to 0° C., and LAH (1 M in THF, 3.71 mL, 3.71 mmol, Sigma-Aldrich, Inc.) was added dropwise. After 5 min the ice bath was removed and the reaction was left stirring at rt for 20 min. The reaction was quenched with H₂O (15 mL) and sat. aq. NaHCO₃ (10 mL), extracted with EtOAc (3×15 mL), dried over MgSO₄, filtered, then concentrated under vacuum. The resulting product was slurried in EtOAc for 1 h, then filtered and the mother liquor was combined with the organic layer from the extraction and concentrated to give 3-(3-methyl-1-(tetrahydro-2H₁-pyran-2-yl)-4-(1,2,3,6-tetrahydropyridin-4-yl)-1H-pyrazol-5-yl)propan-1-ol that was used in the next step with no further purification. m/z (ESI): 306.3 (M+H)⁺.

Step 5. To a glass vial was added 3-(3-methyl-1-(tetrahydro-2H-pyran-2-yl)-4-(1,2,3,6-tetrahydropyridin-4-yl)-1H-pyrazol-5-yl)propan-1-ol (645 mg, 2.112 mmol) and sodium hydroxide (4224 μL, 8.45 mmol, Sigma-Aldrich, Inc.) in THF (8.5 mL), and the mixture was cooled to 0° C. CbzCl (301 μL, 2.112 mmol, Oakwood Products, Inc.) was then added dropwise. After addition. the ice bath was removed, and the reaction was left stirring at rt for 18 h. The reaction was diluted with sat. NaHCO₃ (15 mL), extracted with EtOAc (3×15 mL), dried over MgSO₄, filtered and concentrated. The crude material was absorbed onto a plug of silica gel and purified by chromatography, eluting with a gradient of 0% to 100% EtOAc in heptane, to provide benzyl 4-(5-(3-hydroxypropyl)-3-methyl-1-(tetrahydro-2H-pyran-2-yl)-1H-pyrazol-4-yl)-3,6-dihydropyridine-1(2H)-carboxylate.

Step 6. To a vessel was added benzyl 4-(5-(3-hydroxypropyl)-3-methyl-1-(tetrahydro-2H-pyran-2-yl)-1H-pyrazol-4-yl)-3,6-dihydropyridine-1(2H)-carboxylate (506 mg, 1.151 mmol) and TFA (1313 mg, 887 μL, 11.51 mmol, Apollo) in DCM (5.8 mL), and the mixture was stirred at 40° C. for 2.5 h. The temperature was raised to 50° C., and stirred for 1.5 h. MeOH (5 mL) was added and the mixture was stirred at 50° C. for 2 h. The reaction was concentrated in vacuo. diluted with sat. aq NaHCO₃ (15 mL), extracted with EtOAc (3×15 mL), dried over MgSO₄, filtered and concentrated, to give the crude benzyl 4-(5-(3-hydroxypropyl)-3-methyl-1H-pyrazol-4-yl)-3,6-dihydropyridine-1(2H)-carboxylate, that was used in the next step with no further purification. m/z (ESI): 356.2 (M+H)⁺

Step 7. To a 150-mL round-bottomed flask was added benzyl 4-(5-(3-hydroxy propyl)-3-methyl-1H-pyrazol-4-yl)-3,6-dihydroxyindane-1(2H)-carboxylate (458 mg, 1.289 mmol) and triphenylphosphine (1014 mg, 3.87 mmol, Sigma-Aldrich, Inc.) in THF (12.9 mL), and the mixture was cooled to 0° C. diisopropyl diazene-1,2-dicarboxylate (808 mg, 786 μL, 3.99 mmol, Oakwood Products, Inc.) was then added dropwise to the reaction, and the mixture was then left stirring at RT. After 80 min, the reaction mixture was diluted with sat. NaHCO₃ (15 mL), extracted with EtOAc (3×15 mL), dried over MgSO₄, filtered and concentrated. The crude material was absorbed onto a plug of silica gel and purified by chromatography, eluting with a gradient of 0% to 100% 3:1 EtOAc:EtOH in heptane, to provide benzyl 4-(2-methyl-5,6-dihydro-4H-pyrrolo[1,2-b]pyrazol-3-yl)-3,6-dihydropyridine-1(2H)-carboxylate. m/z (ESI): 338.2 (M+H)⁺.

Step 8. To a vessel was added benzyl 4-(2-methyl-5,6-dihydro-4H-pyrrolo[1,2-b]pyrazol-3-yl)-3,6-dihydropyridine-1(2H)-carboxylate (152 mg, 0.450 mmol) and ammonium formate (284 mg, 4.50 mmol, Sigma-Aldrich, Inc.) in EtOH (4.5 mL). To the mixture was then added acetic acid (129 μL, 2.252 mmol, Sigma-Aldrich. Inc.), palladium on activated carbon (144 mg, 0.135 mmol, Sigma-Aldrich, Inc.) and palladium hydroxide on carbon (95 mg, 0.135 mmol, Combi-Blocks, Inc.) and the reaction was heated to 50° C. for 2 h. The reaction was filtered through a pad of Celite and washed with EtOAc. The filtrate was concentrated to give 2-methyl-3-(1,2,3,6-tetrahydropyridin-4-yl)-5,6-dihydro-4H-pyrrolo[1,2-b]pyrazole, that was used in the next step with no further purification. m/z (ESI): 206.2 (M+H)⁺.

The intermediate in the table below was prepared in a fashion similar to that described above. Synthesis of the building blocks is either described above or from commercial sources.

| | | | m/z (ESI): |
|---|---|---|---|
| Int. # | Chemical Structure | Name | (M + H)⁺ |
| B-108 | 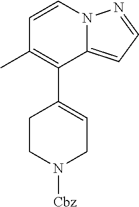 [SMILES: Cc1ccn2nccc2c1C1=CCN(C(=O)OCc2ccccc2)CC1] | benzyl 4-(5-methylpyrazolo[1,5-a]pyridin-4-yl)- 3,6-dihydropyridine-1(2H)-carboxylate | 348.3 |

### Tert-Butyl (2R)-4-(2-cyclopropyl-4-methylpyridin-3-yl)-4-methoxy-2-methylpiperidine-1-carboxylate (Intermediate B-109)

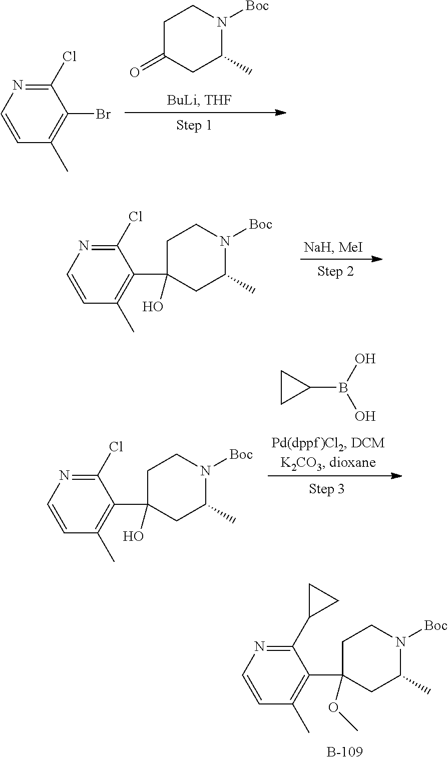

Step 1. To a vessel was added 3-bromo-2-chloro-4-methylpyridine (3 g, 14.53 mmol, ChemScene) and THF (100 mL). The mixture was cooled to −78° C., and n-butyllithium solution (2.5 M in hexanes, 6.4 mL 15.98 mmol, Sigma-Aldrich, Inc.) was added dropwise. The mixture was stirred at −78° C. for 5 min, and then tert-butyl (R)-2-methyl-4-oxopiperidine-1-carboxylate (3.72 g, 17.44 mmol, Combi-Blocks, Inc.) was added. The mixture was warmed to rt over 30 min. The reaction was quenched with H₂O, extracted with EtOAc, and concentrated to give tert-butyl (2R,4S)-4-(2-chloro-4-methylpyridin-3-yl)-4-hydroxy-2-methylpiperidine-1-carboxylate which was used in the following step as is. m/z (ESI): 327.2 (M+H)⁺.

Step 2. To a 250-mL round-bottomed flask was added tert-butyl (2R)-4-(2-chloro-4-methylpyridin-3-yl)-4-hydroxy-2-methylpiperidine-1-carboxylate (4.95 g, 14.52 mmol) and THF (100 mL). The mixture was cooled to −10° C., and sodium hydride (60% dispersion in mineral oil) (1.743 g, 72.6 mmol, TCI America) was added. The mixture was allowed to warm to rt over 20 min, and iodomethane (10.31 g, 72.6 mmol, Sigma-Aldrich. Inc.) was added. The mixture was stirred at rt for 18 h, quenched with water, and extracted with EtOAc. The combined organics were concentrated and chromatographed with 0-50% (3:1 EtOAc:EtOH) in heptane to give tert-butyl 12R)-4-(2-chloro-4-methylpyridin-3-yl)-4-methoxy-2-methylpiperidine-1-carboxylate, which was moved forward as a mixture. m/z (ESI): 355.2 (M+H)⁺.

Step 3. A mixture of tert-butyl (2R)-4-(2-chloro-4-methylpyridin-3-yl)-4-methoxy-2-methylpiperidine-1-carboxylate (864 mg, 2.435 mmol), cyclopropylboronic acid (1046 mg, 12.17 mmol, Combi-Blocks, Inc.), Pd(dppf)Cl₂ DCM (199 mg, 0.243 mmol, Combi-Blocks. Inc.) and K₂CO₃ (1009 mg, 7.30 mmol, Sigma-Aldrich, Inc.) was purged with N₂, before the addition of 1,4-dioxane/H₂O (6/0.6 mL), and the resulting mixture was heated at 105° C. for 3 h. The reaction was concentrated and chromatographed on silica gel with 0-50% (3:1 EtOAc:EtOH) in heptane to give tert-butyl (2R)-4-(2-cyclopropyl-4-methylpyridin-3-yl)-4-methoxy-2-methylpiperidine-1-carboxylate. m/z (ESI): 361.2 (M+H)⁺.

### (2R)-4-(1-(2-Methoxyethyl)-4-methyl-1H-pyrazol-5-yl)-2-methylpiperidine (Intermediate B-150)

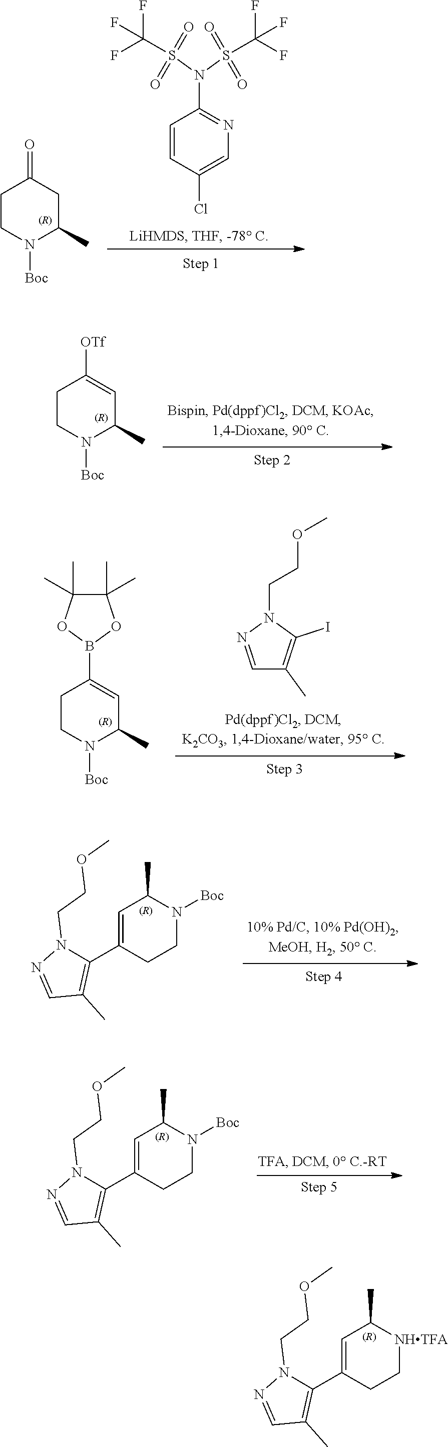

Step 1. To a stirred solution of tert-butyl (R)-2-methyl-4-oxopiperidine-1-carboxylate (25 g, 117 mmol, Combi-Blocks, Inc.) in THF (250 mL) was added LiHMDS (1 M in THF, 176 mL, 176 mmol) at −78° C. under nitrogen atmosphere and slowly brought to 0° C. To this reaction mixture was added N-(5-chloropyridin-2-yl)-1,1,1-trifluoro-N-((trifluoromethyl)sulfonyl)methanesulfonamide (59.8 g, 152 mmol) dropwise and stirred at rt for 30 min. The reaction mass was quenched with satd aqueous NH₄Cl solution (500 mL) and extracted with DCM (2×300 mL). The combined organic extracts were washed with brine solution (200 mL), dried (Na₂SO₄), filtered, and concentrated under reduced pressure to give tert-butyl (R)-6-methyl-4-(((trifluoromethyl)sulfonyl)oxy)-3,6-dihydropyridine-1(2H)-carboxylate (22 g, 63.7 mmol, 54% yield). m/z (ESI): 246.0 (M-Boc)⁺.

Step 2. To a stirred solution of tert-butyl (R)-6-methyl-4-(((trifluoromethyl)sulfonyl)oxy)-3,6-dihydropyridine-1(2H)-carboxylate (11 g, 31.9 mmol) in 1,4-dioxane (100 mL) were added bispinacolato diboron (8.90 g, 35.0 mmol) and potassium acetate (9.38 g, 96 mmol) at rt. The reaction mixture was degassed and purged with nitrogen for 5 min. PdCl₂(dppf)-CH₂Cl₂ (1.301 g, 1.593 mmol) was added and the reaction mixture was stirred at 90° C. for 16 h. The reaction mass was diluted with EtOAc (100 mL). filtered through a celite bed, and washed with EtOAc (50 mL). The filtrate was concentrated under reduced pressure. The crude residue was purified by column chromatography (Redi-sep pre-packed silica gel column, eluting with a gradient of 25% to 30% EtOAc m hexanes) to give tert-butyl (R)-6-methyl-4-(4,4,5,5-tetramethyl-1,3,2-dioxaborolan-2-yl)-3,6-dihydropyridine-1(2H)-carboxylate (8 g, 24.75 mmol, 78% yield). m/z (ESI): 224.0 (M-Boc)⁺. ¹H NMR (400 MHz, DMSO-d₆): δ 6.36 (d, J=22.3 Hz, 1H), 4.33 (s, 1H), 4.16-3.86 (m, 1H), 3.52 (s, 1H), 2.05-1.88 (m, 2H), 1.40 (s, 9H), 1.21 (s, 12H), 1.19 (s, 3H).

Step 3. To a solution of tert-butyl (R)-6-methyl-4-(4,4,5,5-tetramethyl-1,3,2-dioxaborolan-2-yl)-3,6-dihydropyridine-1(2H)-carboxylate (5.10 g, 15.78 mmol) in 1,4-dioxane (56 mL) and water (14 mL) were added 5-iodo-1-(2-methoxyethyl)-4-methyl-1H-pyrazole (3.5 g, 13.15 mmol) and potassium carbonate (5.45 g, 39.5 mmol) at rt. The reaction mixture was degassed and purged with nitrogen for 5 min and PdCl₂(dppf)-CH₂Cl₂ (0.537 g, 0.658 mmol) was added. The reaction mixture was heated to 90° C., and stirred for 16 h. The reaction mixture was quenched with ice cold water (100 mL) and extracted with ethyl acetate (2×40 mL). The combined organic layer was dried (Na₂SO₄), filtered, and concentrated under reduced pressure. The crude residue was purified by column chromatography (Redi-sep pre-packed silica gel column (80 g), eluting with a gradient of 20% to 40% EtOAc in hexanes) to give tert-butyl (R)-4-(1-t 2-methoxyethyl)-4-methyl-1H-pyrazol-5-yl)-6-methyl-3,6-dihydropyridine-1(2H)-carboxylate (3.5 g, 10.43 mmol, 79% yield). m/z (ESI): 336.1 (M+H)⁺. ¹H NMR (400 MHz, DMSO-d₆): δ 7.23 (s, 1H), 5.76 (dt, J=15.7, 3.3 Hz, 1H), 4.49 (s, 1H), 4.30-4.04 (m, 3H), 3.93 (s, 2H), 3.63 (td, J=5.5, 2.0 Hz, 3H), 3.22-3.13 (m, 3H), 2.62 (m, 2H), 2.40 (m, 2H), 1.44 (d, J=1.7 Hz, 9H), 1.24-1.13 (m, 3H).

Step 4. To a stirred solution of tert-butyl (R)-4-(1-(2-methoxyethyl)-4-methyl-1H-pyrazol-5-yl)-6-methyl-3,6-dihydropyridine-1(2H)-carboxylate (3.5 g, 10.43 mmol) in MeOH (70 mL) were added Pd/C (10%, 1.66 g) and palladium hydroxide on carbon (20%, 1.66 g) and the reaction mass was degassed thoroughly and stirred under hydrogen pressure (142 psi) at 50° C. for 16 h. The reaction mass was filtered through a celite bed and washed with MeOH (100 mL). The filtrate was concentrated under reduced pressure to give tert-butyl (2R)-4-(1-(2-methoxyethyl)-4-methyl-1H-pyrazol-5-yl)-2-methylpiperidine-1-carboxylate (3.5 g, 10.37 mmol, 99% yield). The crude was taken for next step without further purification. m/z (ESI): 338.2 (M+H)⁺. ¹H NMR (400 MHz, DMSO-d₆): δ 7.12 (d, J=6.2 Hz, 1H), 4.47-4.07 (m, 3H), 3.93 (s, 2H), 3.66-3.53 (m, 3H), 3.24-3.16 (m, 4H), 3.02-2.84 (m, 1H), 2.10 (s, 3H), 1.89 (dd, J=16.1, 4.6 Hz, 1H), 1.73-1.61 (m, 2H), 1.42 (d, J=4.0 Hz, 9H), 1.18 (t, J=6.9 Hz, 3H).

Step 5. To a stirred solution of tert-butyl (2R)-4-(1-(2-methoxyethyl)-4-methyl-1H-pyrazol-5-yl)-2-methylpiperidine-1-carboxylate (3.5 g, 10.37 mmol) in DCM (35 mL) at 0° C. was added TFA (15.98 mL, 207 mmol) dropwise and the reaction mixture was stirred at rt for 1 h. The reaction mass was concentrated under reduced pressure and azeotroped with toluene (150 mL) followed by trituration with diethyl ether (100 mL) to give (2R)-4-(1-(2-methoxyethyl)-4-methyl-1H-pyrazol-5-yl)-2-methylpiperidine as a trifluoracetate salt (Intermediate B-150) (3.4 g, 10.14 mmol, 98% yield). m/z (ESI): 238.2 (M+H)⁺.

### Piperazine Intermediates

### Tert-Butyl 5-(4-methyl-1-(oxetan-3-yl)-1H-pyrazol-5-yl)-2,5-diazabicyclo[4.1.0]heptane-2-carboxylate (Intermediate C-1)

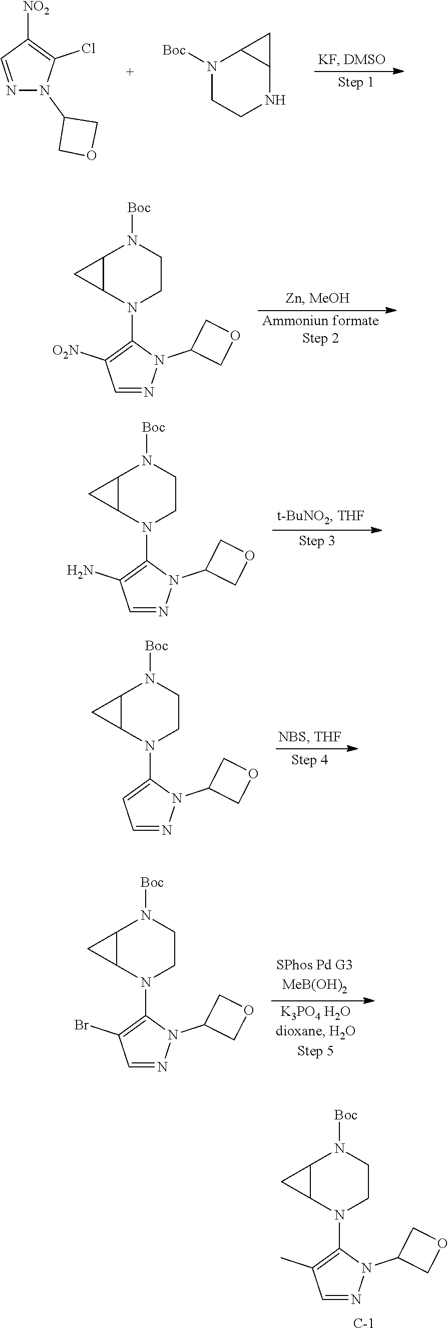

Step 1. A mixture of 5-chloro-4-nitro-1-(oxetan-3-yl)-1N-pyrazole (1.37 g, 6.73 mmol), 2,5-diazabicyclo[4.1.0]heptane-2-carboxylic acid dimethylethyl ester (1.33 mL, 6.73 mmol, Enamine), and potassium fluoride (2.34 g, 40.4 mmol, Sigma-Aldrich. Inc.) in DMSO (15 mL) was heated at 90° C. for 1 h. The reaction was brought to rt, diluted with H₂O, and extracted with EtOAc (3×). The combined organics extracts were dried over Na₂SO₄, filtered, concentrated, and chromatographed on silica gel using 0% to 20% (3:1) EtOAc/EtOH in heptane to afford tert-butyl 5-(4-nitro-1(oxetan-3-yl)-1H-pyrazol-5-yl)-2,5-diazabicyclo[4.1.0]heptane-2-carboxylate. m/z (ESI): 388.2 (M+Na)⁺.

Step 2. To a solution of tert-butyl 5-(4-nitro-1-(oxetan-3-yl)-1H-pyrazol-5-yl)-2,5-diazabicyclo[4.1.0]heptane-2-carboxylate (1.60 g, 4.38 mmol) in MeOH (30 mL) was added ammonium formate (0.828 g, 13.14 mmol, Sigma-Aldrich, Inc.) and zinc dust (0.859 g, 13.14 mmol, Sigma-Aldrich, Inc.) and the resulting mixture was stirred at rt for 2 h. The reaction mixture was filtered through celite and the filtrate was concentrated. The residue obtained was diluted with 3:1) EtOAc/EtOH and washed with H₂O. The aqueous phase was extracted with (3:1) EtOAc/EtOH and the organic extracts were combined, washed with brine (3×), dried over Na₂SO₄, filtered, and concentrated to afford tert-butyl 5-(4-amino-1-(oxetan-3-yl)-1H-pyrazol-5-yl)-2,5-diazabicyclo[4.1.0]heptane-2-carboxylate. The crude material was used directly in the next step. m/z (ESI): 336.3 (M+H)⁺.

Step 3. To a solution of crude tert-butyl 5-(4-amino-1-(oxetan-3-yl)-1H-pyrazol-5-yl)-2,5-diazabicyclo[4.1.0]heptane-2-carboxylate (1.469 g, 4.38 mmol) in THF (25 mL) was added dropwise tert-butyl nitrite (1.050 mL, 8.76 mmol, Sigma-Aldrich, Inc.) and the resulting mixture was heated to 80° C. for 5 h. The reaction was brought to rt, diluted with H₂O, and extracted with EtOAc. The combined organics extracts were dried over Na₂SO₄, filtered. concentrated, and chromatographed on silica gel using 0% to 30% (3.1) EtOAc/EtOH in heptane to afford tert-butyl 5-(1-(oxetan-3-yl)-1H-pyrazol-5-yl)-2,5-diazabicyclo[4.1.0]heptane-2-carboxylate. m/z (ESI): 321.3 (M+H)⁺.

Step 4. A solution of tert-butyl 5-(1-(oxetan-3-yl)-1H-pyrazol-5-yl)-2,5-diazabicyclo[4.1.0]heptane-2-carboxylate (0.604 g, 1.88 mmol) and NBS (0.336 g, 1.885 mmol, Sigma-Aldrich. Inc.) in THF (10 mL) was heated to 80° C., and stirred for 40 min. The reaction mixture was diluted with sat. aq. NH₄Cl and extracted with EtOAc. The organic extract was dried over Na₂SO₄ and concentrated in vacuo. The crude material was absorbed onto a plug of silica gel and purified by chromatography through silica gel eluting with a gradient of 0% to 30% (3:1) EtOAc/EtOH in heptane to afford tert-butyl 5-(4-bromo-1-(oxetan-3-yl)-1H-pyrazol-5-yl)-2,5-diazabicyclo[4.1.0]heptane-2-carboxylate. m/z (ESI): 399.0 (M+H)⁺.

Step 5. A mixture of tert-butyl 5-(4-bromo-1-(oxetan-3-yl)-1H-pyrazol-5-yl)-2,5-diazabicyclo[4.1.0]heptane-2-carboxylate (0.667 g, 1.670 mmol). SPhos Pd G3 (0.289 g, 0.334 mmol, Sigma-Aldrich, Inc.). methylboronic acid (0.400 g, 6.68 mmol, Combi-Blocks. Inc.), potassium phosphate (1.92 g, 8.35 mmol, Sigma-Aldrich, Inc.) was purged with N₂ followed by the addition of 1,4-dioxane/H₂O (10/1.0 mL) and heated at 95° C. for 30 min. Then, the reaction mixture was brought to rt, diluted with H₂O, and extracted with EtOAc. The combined organics were dried over Na₂SO₄, filtered, concentrated, and chromatographed on silica gel using 0% to 30% (3:1)EtOAc/EtOH in heptane to afford tert-butyl 5-(4-methyl-1-(oxetan-3-yl)-1H-pyrazol-5-yl)-2,5-diazabicyclo[4.1.0]heptane-2-carboxylate. m/z (ESI): 335.2 (M+H)⁺.

The intermediates in the table below were prepared in a fashion similar to that described above for intermediate C-1. Synthesis of the building blocks is either described above or from commercial sources.

| | | | m/z (ESI): |
|---|---|---|---|
| Int. # | Chemical Structure | Name | (M + H)⁺ |
| C-2 | 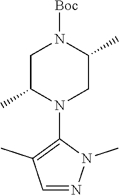 | tert-butyl (2R,5R)-4-(1,4-dimethyl-1H-pyrazol- 5-yl)-2,5-dimethylpiperazine-1-carboxylate | 309.2 |
| C-3 | 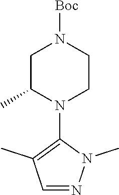 | tert-butyl (R)-4-(1,4-dimethyl-1H-pyrazol-5- yl)-3-methylpiperazine-1-carboxylate | 295.2 |
| C-4 | 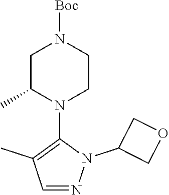 [SMILES: Cc1cnn(C2COC2)c1N1CCN(C(=O)OC(C)(C)C)C[C@H]1C] | tert-butyl (R)-3-methyl-4-(4-methyl-1-(oxetan- 3-yl)-1H-pyrazol-5-yl)piperazine-1-carboxylate | 337.2 |
| C-5 | 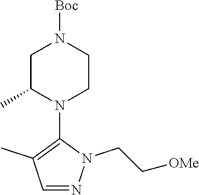 [SMILES: COCCn1ncc(C)c1N1CCN(C(=O)OC(C)(C)C)C[C@H]1C] | tert-butyl (R)-4-(1-(2-methoxyethyl)-4-methyl- 1H-pyrazol-5-yl)-3-methylpiperazine-1- carboxylate | 339.1 |
| C-6 | 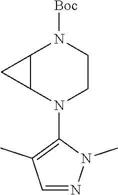 | tert-butyl 5-(1,4-dimethyl-1H-pyrazol-5-yl)- 2,5-diazabicyclo[4.1.0]heptane-2-carboxylate | 293.2 |
| C-17 | 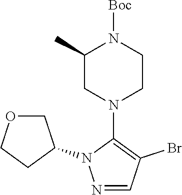 [SMILES: C[C@@H]1CN(c2c(Br)cnn2[C@@H]2CCOC2)CCN1C(=O)OC(C)(C)C] | tert-butyl (R)-4-(4-bromo-1-((R)- tetrahydrofuran-3-yl)-1H-pyrazol-5-yl)-2- methylpiperazine-1-carboxylate | 415.0 |

### Tert-Butyl 4-(4-chloro-1-(oxetan-3-yl)-1H-pyrazol-5-yl)piperazine-1-carboxylate (Intermediate C-7)

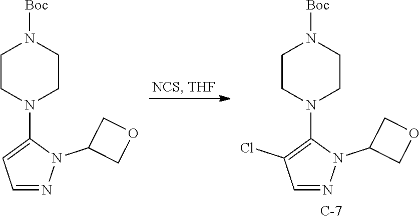

To a solution of tert-butyl 4-(1-(oxetan-3-yl)-1H-pyrazol-5-yl)piperazine-1-carboxylate (2 g, 6.49 mmol) in THF (30 mL) was added NCS (1.299 g, 9.73 mmol) and heated at 80° C. for 3 h. The reaction mixture was concentrated under reduced pressure to give a residue which was purified by column chromatography (SiO₂, 10% to 100% EtOAc in pet ether) to give tert-butyl 4-(4-chloro-1-(oxetan-3-yl)-1H-pyrazol-5-yl)piperazine-1-carboxylate. m/z (ESI): 344.0 (M+H)⁺.

The intermediate in the table below was prepared in a fashion similar to that described above for intermediate C-7 Synthesis of the building blocks is either described above or from commercial sources.

| | | | m/z (ESI): |
|---|---|---|---|
| Int. # | Chemical Structure | Name | (M + H)⁺ |
| C-8 | 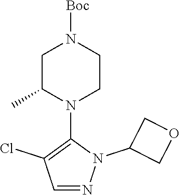 [SMILES: C[C@@H]1CN(C(=O)OC(C)(C)C)CCN1c1c(Cl)cnn1C1COC1] | tert-butyl (R)-4-(4-chloro-1-(oxetan-3-yl)-1H- pyrazol-5-yl)-3-methylpiperazine-1-carboxylate | 357.0 |

### Tert-Butyl 4-(1,4-dimethyl-1H-pyrazol-5-yl)piperazine-1-carboxylate (Intermediate C-9)

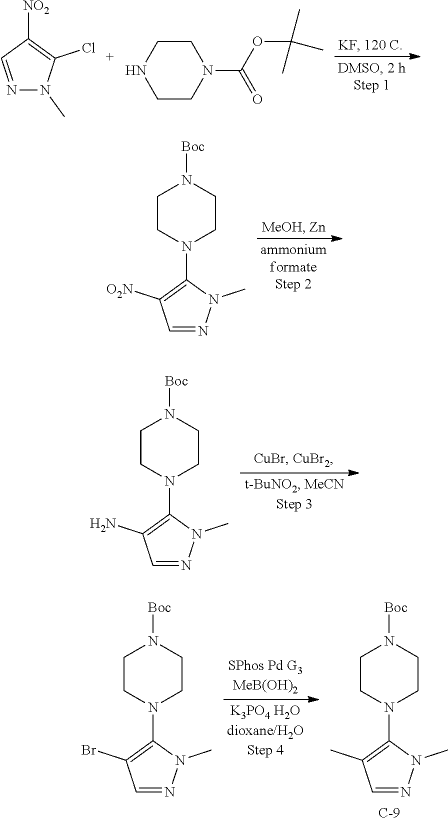

Step 1. A mixture of tert-butyl piperazine-1-carboxylate (0.614 g, 3.30 mmol, Combi-Blocks. Inc.), 5-chloro-1-methyl-4-nitro-1H-pyrazole (0.507 g, 3.14 mmol), and potassium fluoride (1.094 g, 18.83 mmol, Sigma-Aldrich, Inc.) in DMSO (15 mL) was heated at 115° C. for 1 h. The reaction mixture was brought to rt, diluted with H₂O, and extracted with EtOAc. The combined organics extracts were dried over Na₂SO₄, filtered, concentrated, and chromatographed on silica gel using 0% to 20% (3:1) EtOAc/EtOH in heptane to afford tert-butyl 4-(1-methyl-4-nitro-1H-pyrazol-5-yl)piperazine-1-carboxylate. m/z (ESI): 312.2 (M+H)⁺.

Step 2. To a solution of tert-butyl 4-(1-methyl-4-nitro-1H-pyrazol-5-yl)piperazine-1-carboxylate (0.867 g, 2.78 mmol) in MeOH (20 mL) was added zinc dust (0.546 g, 8.35 mmol, Sigma-Aldrich. Inc.) followed by ammonium formate (0.527 g, 8.35 mmol, Sigma-Aldrich, Inc.) and the resulting mixture was stirred at rt for 1 h. Then, the reaction mixture was filtered through celite and the filtrate was concentrated. The residue was diluted with (3:1) EtOAc/EtOH and washed with H₂O. The aqueous phase was extracted with (3:1) EtOAc/EtOH and the organic extracts were combined, washed with brine (2×), dried over Na₂SO₄, filtered, and concentrated to afford tert-butyl 4-(4-amino-1-methyl-1H-pyrazol-5-yl)piperazine-1-carboxylate which was azeotropically dried with toluene m/z (ESI): 282.2 (M+H)⁺.

Step 3. To a suspension of tert-butyl 4-(4-amino-1-methyl-1H-pyrazol-5-yl)piperazine-1-carboxylate (0.784 g, 2.79 mmol), copper (II) bromide (0.622 g, 2.79 mmol, Sigma-Aldrich, Inc.), and copper bromide (0.400 g, 2.79 mmol, Sigma-Aldrich, Inc.) in MeCN (20 mL) was added dropwise tert-butyl nitrite (0.501 mL, 4.18 mmol, Sigma-Aldrich. Inc.) and the resulting mixture was heated at 80° C. for 6 h. The reaction was brought to rt, diluted with sat. aq. NH₄Cl and extracted with EtOAc. The combined organics extracts were dried over Na₂SO₄, filtered. concentrated, and chromatographed on silica gel using 0% to 30% (3:1) EtOAc/EtOH in heptane to afford tert-butyl 4-(4-bromo-1-methyl-1H-pyrazol-5-yl)piperazine-1-carboxylate. m/z (ESI): 346.0 (M+H)⁺.

Step 4. A mixture of tert-butyl 4-(4-bromo-1-methyl-1H-pyrazol-5-yl)piperazine-1-carboxylate (0.355 g, 1.028 mmol), methylboronic acid (0.246 g, 4.11 mmol, Combi-Blocks, Inc.), SPhos Pd G3 (0.160 g, 0.206 mmol, Sigma-Aldrich. Inc.) and potassium phosphate monohydrate (1.184 g, 5.14 mmol, Sigma-Aldrich, Inc.) was purged with N₂, followed by the addition of degassed 1,4-dioxane/H₂O (10/1.0 mL) and the resulting mixture was heated at 95° C. for 30 min. The reaction mixture was brought to rt. washed with H₂O, and extracted with EtOAc. The combined organics extracts were dried over Na₂SO₄, filtered, concentrated, and chromatographed on silica gel using 0% to 20% (3-1) EtOAc/EtOH in heptane to afford tert-butyl 4-(1,4-dimethyl-1H-pyrazol-5-yl)piperazine-1-carboxylate. m/z (ESI): 281.1 (M+H)⁺.

The intermediates in the table below were prepared in a fashion similar to that described above for intermediate C-9. Synthesis of the building blocks is either described above or from commercial sources.

| | | | m/z (ESI): |
|---|---|---|---|
| Int. # | Chemical Structure | Name | (M + H)⁺ |
| C-10 | 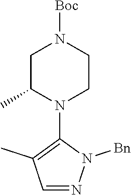 [SMILES: Cc1cnn(Cc2ccccc2)c1N1CCN(C(=O)OC(C)(C)C)C[C@H]1C] | tert-butyl (R)-4-(1-benzyl-4-methyl-1H- pyrazol-5-yl)-3-methylpiperazine-1-carboxylate | 371.1 |
| C-11 | 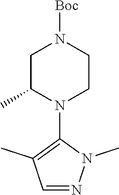 | tert-butyl (R)-4-(1,4-dimethyl-1H-pyrazol-5- yl)-3-methylpiperazine-1-carboxylate | 295.2 |

### Tert-Butyl (R)-3-methyl-4-(4-methyl-1-(oxetan-3-yl)-1H-pyrazol-5-yl)piperazine-1-carboxylate (Intermediate C-12)

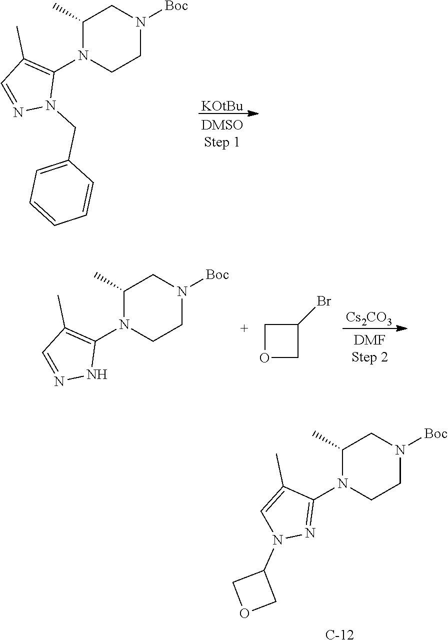

Step 1. To a solution of tert-butyl (R)-4-(1-benzyl-4-methyl-1H-pyrazol-5-yl)-3-methylpiperazine-1-carboxylate (0.357 g, 0.964 mmol) in DMSO (3 mL) was added potassium tert-butoxide solution (6.74 mL, 6.74 mmol, 1.0 M in THF, Sigma-Aldrich, Inc.) and the reaction mixture was stirred at rt. After 5 h, the reaction was diluted with H₂O and extracted with EtOAc. The combined organics extracts were dried over Na₂SO₄, filtered, and concentrated to afford tert-butyl (R)-3-methyl-4-(4-methyl-1H-pyrazol-5-yl)piperazine-1-carboxylate that was used without further purification. m/z (ESI): 281.2 (M+H)⁺.

Step 2. To a solution of ten-butyl (R)-3-methyl-4-(4-methyl-1H-pyrazol-5-yl)piperazine-1-carboxylate (0.270 g, 0.963 mmol) in DMF (10 mL) was added cesium carbonate (0.941 g, 2.89 mmol, Sigma-Aldrich, Inc.) and 3-bromooxetane (0.145 g, 1.059 mmol, Combi-Blocks, Inc.) and the mixture was stirred for 16 h at 100° C. The reaction was brought to rt, diluted with H₂O (25 mL), and extracted with EtOAc. The combined organics extracts were washed with brine, dried over Na₂SO₄, filtered, and concentrated to afford tert-butyl (R)-3-methyl-4-(4-methyl-1-(oxetan-3-yl)-1H-pyrazol-5-yl)piperazine-1-carboxylate to be used as is. m/z (ESI): 337.2 (M+H)⁺.

### Tert-Butyl (2R,5R)-2,5-dimethyl-4-(1,3,4-trimethyl-1H-pyrazol-5-yl)piperazine-1-carboxylate and tert-butyl (2R,5R)-2,5-dimethyl-4-(1,4,5-trimethyl-1H-pyrazol-3-yl)piperazine-1-carboxylate (Intermediates C-13 and C-14)

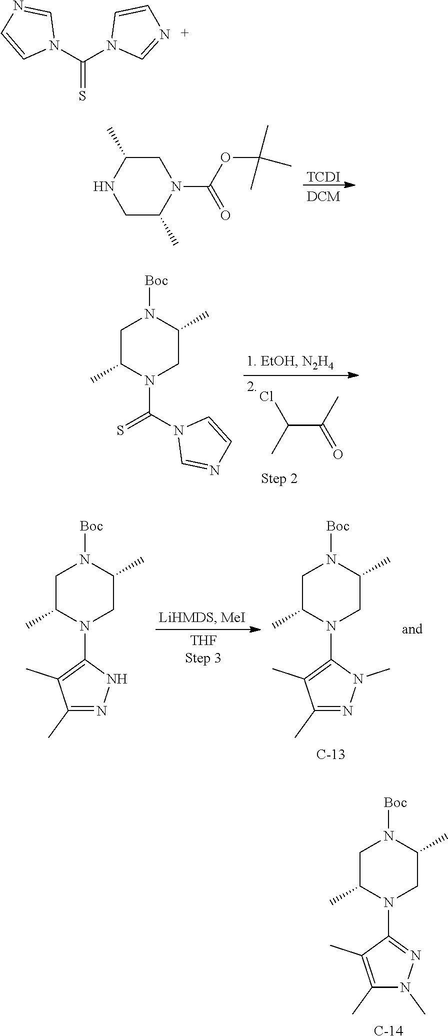

Step 1. To a solution of (2R,5R)-tert-butyl 2,5-dimethylpiperazine-1-carboxylate (1.0 g, 4.67 mmol, Ambeed, Inc.) in DCM (10 mL) was added 1,1′-thiocarbonyldiimidazole (0.832 g, 4.67 mmol, Sigma-Aldrich, Inc.) and the resulting mixture was stirred at rt for 2 h. The reaction was washed with H₂O, dried over Na₂SO₄, filtered, concentrated, and chromatographed on silica gel using 0% to 30% (3:1) EtOAc/EtOH in heptane to afford tert-butyl (2R,5R)-4-(1H-imidazole-1-carbonothioyl)-2,5-dimethylpiperazine-1-carboxylate. m/z (ESI): 325.2 (M+H)⁺.

Step 2. To a solution of tert-butyl (2R,5R)-4-(1H-imidazole-1-carbonothioyl)-2,5-dimethylpiperazine-1-carboxylate (4.67 mmol) in EtOH (10 mL) was added hydrazine monohydrate (0.249 mL, 5.13 mmol, Sigma-Aldrich, Inc.) and the resulting mixture was heated to reflux for 1 h. The reaction was brought to rt, 3-chloro-2-butanone (0.547 mL, 5.13 mmol, Sigma-Aldrich, Inc.) was added dropwise and the resulting mixture was heated at reflux for 7 h. The mixture was brought to rt, concentrated, and chromatographed on silica gel using 0% to 40% (3:1) EtOAc/EtOH in heptane to afford tert-butyl (2R,5R)-4-(3,4-dimethyl-1H-pyrazol-5-yl)-2,5-dimethylpiperazine-1-carboxylate. m/z (ESI): 309.2 (M+H)⁺.

Step 3. To a solution of tert-butyl (2R,5R)-4-(3,4-dimethyl-1H-pyrazol-5-yl)-2,5-dimethylpiperazine-1-carboxylate (0.420 g, 1.362 mmol) in THF (10 mL) at 0° C. was added dropwise LiHMDS (1.50 mL, 1.5 mmol, 1.0 M in THF, Sigma-Aldrich, Inc.) and the resulting mixture was stirred at 0° C. for 30 min. Then iodomethane (0.254 mL, 4.09 mmol, Sigma-Aldrich. Inc.) was added and the stirring at 0° C. was continued for 1 h. The reaction was quenched with sat. aq. NH₄Cl and extracted with EtOAc. The organic extract was dried over Na₂SO₄, filtered, concentrated, and chromatographed on silica gel using 0% to 10% (3:1) EtOAc/EtOH in heptane and repurified by HPLC [10-50% CH₃CN/H₂O (0.1% TFA) over 15 min] to afford tert-butyl (2R,5R)-2,5-dimethyl-4-(1,3,4-trimethyl-1H-pyrazol-5-yl)piperazine-1-carboxylate with <10% of the other regioisomer. m/z (ESI): 323.2 (M+H)⁺ and tert-butyl (2R,5R)-2,5-dimethyl-4-(1,4,5-trimethyl-1H-pyrazol-3-yl)piperazine-1-carboxylate. m/z (ESI): 323.2 (M+H)⁺.

### 5-((2R,5R)-2,5-Dimethylpiperazin-1-yl)-4-methylthiazole hydrochloride (Intermediate C-15)

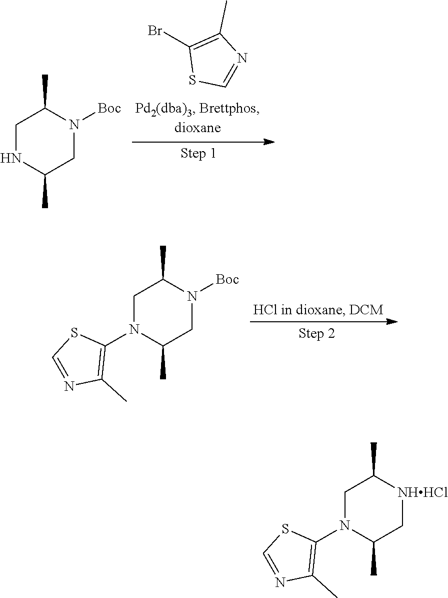

Step 1. To a solution of tert-butyl (2R,5R)-2,5-dimethylpiperazine-1-carboxylate (1.5 g, 7.00 mmol, Combi-Blocks, Inc.) in 1,4-dioxane (10 mL) (degassed) in a scaled tube was added 5-bromo-4-methylthiazole (1.495 g, 8.40 mmol, Chempure), BrettPhos (0.375 g, 0.700 mmol, Combi-Blocks, Inc.), sodium tert-butoxide (2.69 g, 28.0 mmol, Chempure) and Pd₂(dba)₃ (0.320 g, 0.350 mmol. Hindustan Platinum) and the reaction mixture was stirred at 70° C. for 16 h. The reaction mixture was quenched with ice cold water (50 mL) and extracted with EtOAc (2×75 mL). The combined organic extracts were washed with brine (100 mL), dried over Na₂SO₄, filtered, and concentrated under reduced pressure. The crude residue was purified by chromatography (silica, 0% to 45% EtOAc:hexanes) to give tert-butyl (2R,5R)-2,5-dimethyl-4-(4-methylthiazol-5-yl)piperazine-1-carboxylate. m/z (ESI): 312.3 (M+H)⁺. ¹H NMR (400 MHz, DMSO-d₆) δ ppm 8.86 (s, 1H), 4.18 (s, 1H), 3.80 (d, 1H, J=12.9 Hz), 2.6-2.8 (m, 4H), 2.28 (s, 3H), 1.42 (s, 9H), 1.28 (d, 3H, J=6.8 Hz), 0.80 (d, 3H, J=6.1 Hz).

Step 2. To a solution of tert-butyl (2R,5R)-2,5-dimethyl-4-(4-methylthiazol-5-yl)piperazine-1-carboxylate (1.7 g, 5.46 mmol) in DCM (17.0 mL) was added HCl (4 mL, 16 mmol, 4.0 M in dioxane) at 0° C., and the reaction mixture was stirred at rt for 2 h. The reaction mixture was co-evaporated with toluene and triturated with diethyl ether (25 mL) to give crude 5-((2R,5R)-2,5-dimethylpiperazin-1-yl)-4-methylthiazole hydrochloride salt. m/z (ESI): 212.0 (M+H)⁺.

### Tert-Butyl (1R,2S,5S)-3-(1,4-dimethyl-1H-pyrazol-5-yl)-2-methyl-3,8-diazabicyclo[3.2.1]octane-8-carboxylate (Intermediate C-16)

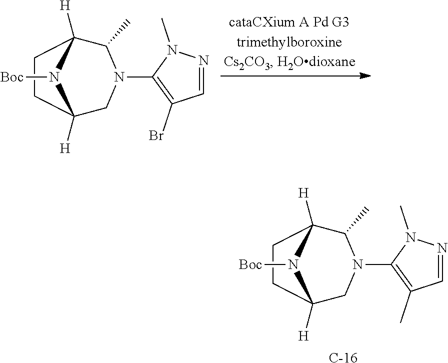

A vessel was charged with tert-butyl (1R,2S,5S)-3-(4-bromo-1-methyl-1H-pyrazol-5-yl)-2-methyl-3,8-diazabicyclo[3.2.1]octane-8-carboxylate (13 mg, 0.034 mmol). cataCXium A Pd G3 (2.457 mg, 3.37 μmol, Sigma-Aldrich. Inc.), 1,4-dioxane (1 mL) and H₂O (0.1 mL) followed by trimethylboroxine (6.35 mg, 7.07 μL, 0.051 mmol, Sigma-Aldrich. Inc.). The reaction mixture was sparged with argon for 5 min before the addition of Cs₂CO₃ (22 mg, 0.067 mmol). The reaction mixture was heated to 100° C. for 2 h. The reaction mixture was diluted with EtOAc, dried over MgSO₄ and concentrated in vacuo. The crude material was dissolved in DCM and purified by chromatography through a 10 g Biotage afar HC D Column, eluting with a gradient of 5% to 100% EtOAc in heptane, then redissolved in DCM and purified by chromatography through a 11 g Biotage sfAr Amino D Column, eluting with a gradient of 5% to 100%/6 EtOAc in heptane. The resulting material was redissolved in DMSO and purified by chromatography through a 12 g Biotage sfar C18 D Column, eluting with a gradient of 5% to 100% MeCN+0.1% HCOOH in H₂O+0.1% HCOOH to provide tert-butyl (1R,2S,5S)-3-(1,4-dimethyl-1H-pyrazol-5-yl)-2-methyl-3,8-diazabicyclo[3.2.1]octane-8-carboxylate. m/z (ESI): 321.3 (M+H)⁺. ¹H NMR (400 MHz, CHLOROFORM-d) δ ppm 7.30 (s, 1H) 4.17-4.37 (m, 1H) 3.87-4.09 (m, 1H) 3.81 (s, 3H) 3.41-3.60 (m, 1H) 3.14-3.36 (m, 1H) 2.59-2.66 (m, 1H) 2.02 (s, 3H) 1.74-1.95 (m, 4H) 1.51 (s, 9H) 0.79 (d, J=6.56 Hz, 3H)

### Linker

### Tert-Butyl 4-((2R,3R)-2-methyl)azetidin-3-yl)piperazine-1-carboxylate (Intermediate D-1)

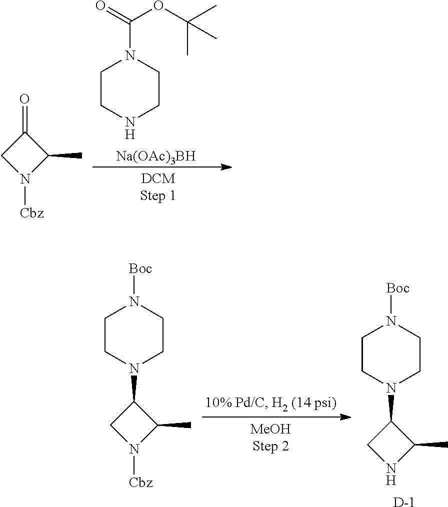

Step 1. To a mixture of benzyl (R)-2-methyl-3-oxoazetidine-1-carboxylate (250 g, 1140 mmol. PharmaBlock, Inc) and tert-butyl piperazine-1-carboxylate (319 g, 1710 mmol, Avra Synthesis) in DCM (7.5 L) at 0° C. was added sodium triacetoxyborohydride (725 g, 3421 mmol, Avra Synthesis) and the reaction mixture was stirred at rt for 3 h. The reaction mixture was quenched with H₂O (3 L) and extracted with DCM (3 L). The organic extract was washed with brine (1 L), dried over Na₂SO₄, filtered, concentrated, and purified by chromatography (silica, 15% to 25% EtOAc in hexanes) to give tert-butyl 4-((2R,3R)-1-((benzyloxy)carbonyl)-2-methyl)azetidin-3-yl)piperazine-1-carboxylate. m/z (ESI): 390.3 (M+H)⁺. ¹H NMR (400 MHz, DMSO-do) 5 ppm 7.2-7.5 (m, 5H), 5.03 (s, 2H), 4.30 (s, 1H), 3.6-3.8 (m, 2H), 3.33 (br s, 4H), 3.12 (q, 1H, J=7.2 Hz), 2.17 (d, 4H, J=5.2 Hz), 1.39 (s, 9H), 1.32 (d, 3H, J=6.4 Hz).

Step 2. To a solution of tert-butyl 4-((2R,3R)-1-((benzyloxy)carbonyl)-2-methyl)azetidin-3-yl)piperazine-1-carboxylate (100 g, 257 mmol) in MeOH (1 L) was added 10% palladium on carbon (38.3 g, Sigma-Aldrich, Inc.) and the reaction mixture was stirred under hydrogen atmosphere (14 psi) at rt for 48 h. The reaction mixture was filtered over a pad of celite and washed with MeOH. The filtrate was concentrated to give tert-butyl 4-((2R,3R)-2-methyl)azetidin-3-yl)piperazine-1-carboxylate. m/z (ESI): 256.2 (M+H)⁺. ¹H NMR (400 MHz, DMSO-d₆) δ ppm 3.5-3.7 (m, 1H), 3.2-3.4 (m, 6H), 2.99 (q, 1H, J=7.4 Hz), 2.0-2.1 (m, 41), 1.39 (s, 9H), 1.23 (d, 3H, J=6.5 Hz).

The intermediates in the table below were prepared in a fashion similar to that described above for intermediate D-1. Synthesis of the building blocks is either described above or from commercial sources.

| | | | m/z (ESI): |
|---|---|---|---|
| Int. # | Chemical Structure | Name | (M + H)⁺ |
| D-2 | 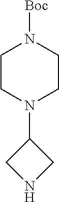 [SMILES: CC(C)(C)OC(=O)N1CCN(C2CNC2)CC1] | tert-butyl 4-(azetidin-3-yl)piperazine-1- carboxylate | 242.1 |
| D-3 | 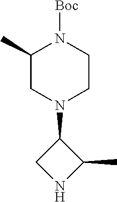 | tert-butyl (R)-2-methyl-4-((2R,3R)-2- methylazetidin-3-yl)piperazine-1-carboxylate | 270.1 |
| D-4 | 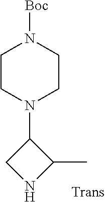 [SMILES: CC1NCC1N1CCN(C(=O)OC(C)(C)C)CC1] | tert-butyl 4-(2-methylazetidin-3-yl)piperazine- 1-carboxylate | 256.4 |
| D-11 | 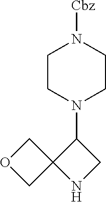 [SMILES: O=C(OCc1ccccc1)N1CCN(C2CNC23COC3)CC1] | benzyl 4-(6-oxa-1-azaspiro[3.3]heptan-3- yl)piperazine-1-carboxylate | 318.1 |
| D-12 | 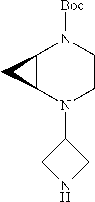 [SMILES: CC(C)(C)OC(=O)N1CCN(C2CNC2)[C@H]2C[C@H]21] | tert-butyl (1R,6S)-5-(azetidin-3-yl)-2,5- diazabicyclo[4.1.0]heptane-2-carboxylate | 254.1 |
| D-13 | 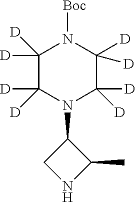 [SMILES: [2H]C1([2H])N(C(=O)OC(C)(C)C)C([2H])([2H])C([2H])([2H])N([C@@H]2CN[C@@H]2C)C1([2H])[2H]] | tert-butyl 4-((2R,3R)-2-methylazetidin-3- yl)piperazine-1-carboxylate-2,2,3,3,5,5,6,6-d8 | 264.2 |
| D-14 | 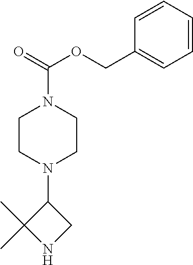 [SMILES: CC1(C)NCC1N1CCN(C(=O)OCc2ccccc2)CC1] | benzyl 4-(2,2-dimethylazetidin-3-yl)piperazine- 1-carboxylate | 304.0 |
| D-15 | 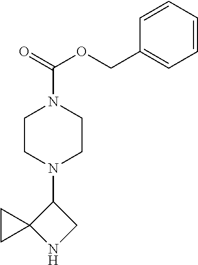 [SMILES: O=C(OCc1ccccc1)N1CCN(C2CNC23CC3)CC1] | benzyl 4-(4-azaspiro[2.3]hexan-6- yl)piperazine-1-carboxylate | 302.0 |

### (2S)-1-Benzyl-2-(fluoromethyl)-4-((2R)-2-methyl)azetidin-3-yl)piperazine trifluoroacetate (Intermediate D-5)

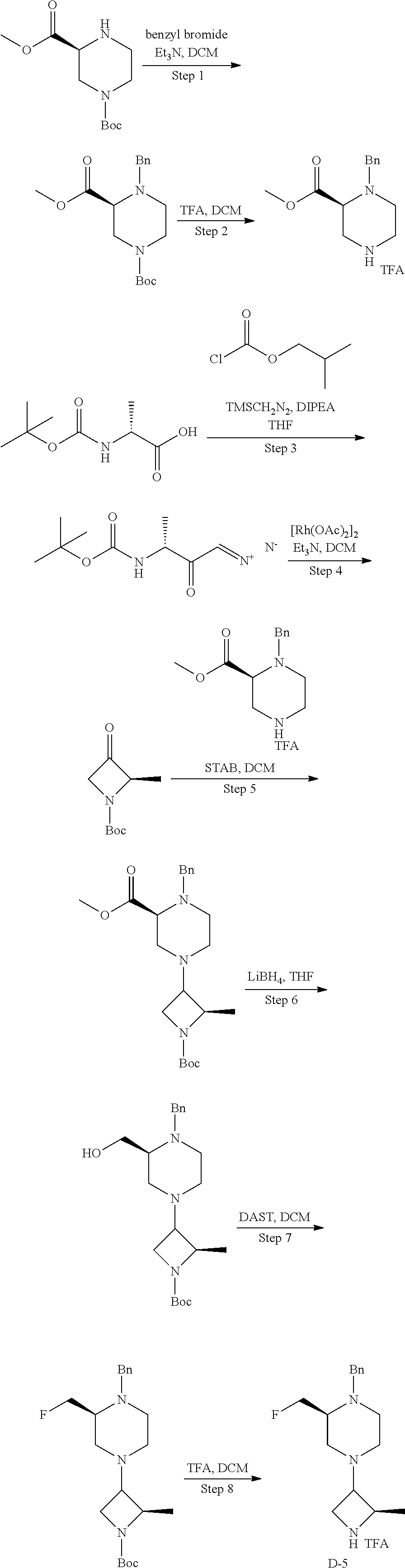

Step 1. To a stirred solution of 1-(tert-butyl) 3-methyl (S)-piperazine-1,3-dicarboxylate (10.0 g, 40.9 mmol, Combi-Blocks. Inc.) in DCM (100 mL) was added TEA (11.41 mL, 82 mmol, Spectrochem) followed by (bromomethyl)benzene (5.35 mL, 45.0 mmol, Avra) at 0° C., and the reaction mixture was stirred at 25° C. for 16 ht. The reaction mixture was quenched with ice cold water (50 mL) and extracted with DCM (2×100 mL). The combined organic extracts were washed with brine (50 mL) and dried over anhydrous Na₂SO₄. The solution was filtered. concentrated under reduced pressure, and purified by column chromatography (silica, 20% to 30% EtOAc in hexanes) to obtain 1-(tert-butyl) 3-methyl (S)-4-benzylpiperazine-1,3-dicarboxylate. m/z (ESI): 335.1 (M+H)⁺. ¹H NMR (400 MHz, DMSO-d₆) δ ppm 7.2-7.4 (m, 5H), 3.7-4.0 (m, 2H), 3.5-3.7 (m, 5H), 3.3-3.4 (m, 2H), 2.9-3.2 (m, 2H), 2.2-2.4 (m, 1H), 1.38 (s, 9H).

Step 2. To stirred solution of 1-(tert-butyl) 3-methyl (S)-4-benzylpiperazine-1,3-dicarboxylate (2.5 g, 7.48 mmol) in DCM (20 mL), was added TFA (11.52 mL, 150 mmol, Spectrochem) at 0° C. and the reaction mixture was stirred at rt for 1 h. The reaction mixture was concentrated under reduced pressure to obtain methyl (S)-1-benzylpiperazine-2-carboxylate trifluoroacetate salt. This material was used for the next step without further purification. m/z (ESI): 235.2 (M+H)⁺.

Step 3. To a stirred solution of (tert-butoxycarbonyl)-D-alanine (100 g, 529 mmol, Chempure) in THF (1000 mL) was added DIPEA (157 mL, 898 mmol, Spectrochem) followed by isobutyl carbonochloridate (68.7 mL, 529 mmol, Spectrochem) dropwise at 0° C., and the reaction mixture was stirred for 4 h. Then trimethylsilyldiazomethane (529 mL, 1057 mmol, Chempure) was added to the above reaction mixture at 0° C., and stirred for 2 h. The reaction mixture was warmed to rt and stirred for an additional 6 h. The reaction mixture was quenched with ice cold water (200 mL) and extracted with EtOAc (2×500 mL). The combined organic extracts were washed with H₂O (300 mL), brine (200 mL). dried over anhydrous Na₂SO₄, filtered, and concentrated under reduced pressure. The crude residue was purified by chromatography (silica, 30% to 50% EtOAc:hexanes) to obtain tert-butyl (R)-(4-diazo-3-oxobutan-2-yl)carbamate. m/z (ESI): Not ionized. ¹H NMR (400 MHz, DMSO-d₆) δ ppm 7.25 (d, 1H, J=7.2 Hz), 6.02 (s, 1H), 3.9-4.0 (m, 1H), 1.40 (s, 9H), 1.16 (d, 3H, J=7.2 Hz).

Step 4. A 500 mL multi-neck round-bottomed flask was loaded with tert-butyl (R)-(4-diazo-3-oxobutan-2-yl)carbamate (28.0 g, 131 mmol) and DCM (560 mL) under a nitrogen atmosphere. The reaction flask was covered with aluminum foil and cooled to 0° C. Triethylamine (18.20 mL, 131 mmol. Sonia Industries) was added to the reaction mixture. followed by rhodium(II)acetate dimer (2.90 g, 6.57 mmol) at 0° C. The reaction mixture was stirred for 16 h at it. The reaction mixture was concentrated under reduced pressure (<40° C.) and the crude residue was purified by chromatography (silica, 15% to 20% EtOAc:hexanes) to obtain tert-butyl (R)-2-methyl-3-oxoazetidine-1-carboxylate. m/z (ESI): Not ionized. ¹H NMR (400 MHz, Chloroform-d) 5 ppm 4.9-5.0 (m, 1H), 4.71 (d, 1H, J=16.6 Hz), 4.58 (dd, 1H, J=16.6, 4.3 Hz), 1.51 (s, 9H), 1.47 (d, 3H, J=7.0 Hz).

Step 5. To a 100 mL round-bottomed flask. were added tert-butyl (R)-2-methyl-3-oxoazetidine-1-carboxylate (2.6 g, 14.04 mmol) and (S)-1-benzylpiperazine-2-carboxylate trifluoroacetate salt (4.89 g, 14.04 mmol) in DCM (50 mL) and cooled to 0° C. Then sodium triacetoxyborohydride (11.90 g, 56.1 mmol, Spectrochem) was added portion wise and stirred for 16 h at 25° C. The reaction mixture was quenched with sat. aq. NaHCO₃ (50 mL) and extracted with DCM (2×50 mL). The combined organic extracts were dried over anhydrous Na₂SO₄, filtered, and concentrated under reduced pressure. The crude residue was purified by chromatography (silica, 20% to 30% EtOAc:hexanes) to obtain methyl 1-benzyl-4-(2R)-1-(tert-butoxycarbonyl)-2-methyl)azetidin-3-yl)piperazine-2-carboxylate. m/z (ESI): 404.3 (M+H)⁺.

Step 6. To a stirred solution of methyl 1-benzyl-44(2R)-1-(tert-butoxy)carbonyl)-2-methyl)azetidin-3-yl)piperazine-2-carboxylate (0.4 g, 0.991 mmol) in THF (5 mL) at 0° C. was added a solution of LiBH₄ (2 M in THF, 2.48 mL, 4.96 mmol) dropwise. The reaction mixture was stirred at 25° C. for 16 h. The reaction mixture was cooled to 0° C., quenched with sat. aq. NH₄Cl solution (50 mL), extracted with EtOAc (2×50 mL), washed with brine (50 mL), dried over anhydrous Na₂SO₄, and concentrated. The crude residue was purified by chromatography (silica, 40 to 50% EtoAc:hexanes) to obtain tert-butyl (2R)-3-(4-benzyl-3-(hydroxymethyl)piperazin-1-yl)-2-methyl)azetidine-1-carboxylate. m/z (ESI): 376.4 (M+H)⁺.

Step 7. To the stirred solution of tert-butyl (2R)-3-((S)-4-benzyl-3-(hydroxymethyl)piperazin-1-yl)-2-methyl)azetidine-1-carboxylate (1.2 g, 3.20 mmol) in DCM (12 mL) at −78° C. was added DAST (1.27 mL, 9.59 mmol, Sigma-Aldrich. Inc.) dropwise and the reaction mixture was stirred at rt for 16 h. The reaction mixture was cooled to 0° C., quenched with sat. aq. NaHCO₃ (10 mL), and extracted with DCM (3-20 mL). The combined organic extracts were dried over Na₂SO₄, filtered, and concentrated under reduced pressure. The crude residue was purified by chromatography (silica, 40% to 50% EtOAc:hexanes) to obtain tert-butyl (2R)-3-((S)-4-benzyl-3-(fluoromethyl)piperazin-1-yl)-2-methyl)azetidine-1-carboxylate. m/z (ESI): 378.3 (M+H)⁺

Step 8. To a 50 mL round-bottomed flask was added ter-butyl (2R)-3-((S)-4-benzyl-3-(fluoromethyl)piperazin-1-yl)-2-methyl)azetidine-1-carboxylate (0.35 g, 0.927 mmol) and DCM (3.5 mL) and the reaction mixture was cooled to 0° C. TFA (0.72 mL, 9.27 mmol, Spectrochem) was added dropwise and the resultant mixture was stirred at 25° C. for 2 h. The reaction mixture was concentrated under reduced pressure to give (2S)-1-benzyl-2-(fluoromethyl)-4-((2R)-2-methyl)azetidin-3-yl)piperazine trifluoroacetate salt, which was directly used in the next step without further purification. m/z (ESI): 278.1 (M+H)⁺

The intermediates in the table below were prepared in a fashion similar to that described above for intermediate D-4. Synthesis of the building blocks is either described above or from commercial sources.

| | | | m/z (ESI): |
|---|---|---|---|
| Int. # | Chemical Structure | Name | (M + H)⁺ |
| D-6 | 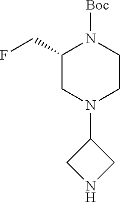 [SMILES: CC(C)(C)OC(=O)N1CCN(C2CNC2)C[C@@H]1CF] | (R)-4-(azetidin-3-yl)-1-benzyl-2- (fluoromethyl)piperazine | 264.1 |
| D-7 | 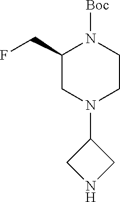 [SMILES: CC(C)(C)OC(=O)N1CCN(C2CNC2)C[C@H]1CF] | (S)-4-(azetidin-3-yl)-1-benzyl-2- (fluoromethyl)piperazine | 264.1 |

### Tert-Butyl (R)-4-(azetidin-3-yl)-2-methylpiperazin-1-carboxylate (Intermediate D-8)

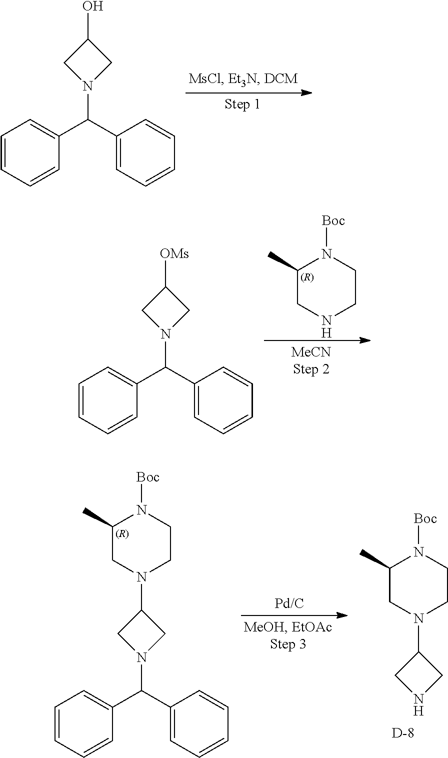

Step 1. To a solution of 1-benzhydryl)azetidin-3-ol (10 g, 41.8 mmol, Combi-Blocks, Inc.) and triethylamine (8.74 mL, 62.7 mmol) in DCM (102 mL), was added at 0° C. methanesulfonyl chloride (3.71 mL, 46.0 mmol, Spectrochem), and the reaction mixture was stirred at it for 2 h. The reaction mixture was quenched with H₂O (200 mL) and extracted with DCM (2×500 mL). The combined organic extracts were washed with brine (150 mL) and dried over Na₂SO₄. The solution was filtered, concentrated, and dried under vacuum to give 1-benzhydryl)azetidin-3-yl methanesulfonate. m/z (ESI): 318.1 (M+H)⁺. ¹H NMR (400 MHz, DMSO-d₆) δ ppm 7.3-7.4 (m, 4H), 7.29 (dd, 4H, J=8.4, 6.8 Hz), 7.1-7.2 (m, 2H), 5.1-5.2 (m, 1H), 4.53 (s, 1H), 3.4-3.5 (m, 2H), 3.19 (s, 3H), 3.0-3.1 (m, 2H).

Step 2. 1-Benzhydryl)azetidin-3-yl methanesulfonate (6.5 g, 20.48 mmol) and tert-butyl (R)-2-methylpiperazine-1-carboxylate (41.0 g, 205 mmol, Combi-Blocks, Inc.) in MeCN (70 mL) were stirred at 80° C. for 19 h. The reaction mixture was concentrated to half volume and the product obtained was filtered and washed with MeCN (100 mL). The filtrate was concentrated to half volume, diluted with H₂O (1 L), and extracted with DCM (2-500 mL). The combined organic extracts were washed with brine (20 mL), dried over Na₂SO₄, filtered, and concentrated. The crude residue was purified by column chromatography (silica, 50 to 60% EtOAc:hexanes) to give tert-butyl (R)-4-(1-benzhydryl)azetidin-3-yl)-2-methylpiperazine-1-carboxylate. m/z (ESI): 422.3 (M+H)⁺. ¹H NMR (400 MHz, DMSO-d₆) δ ppm 7.4-7.5 (m, 4H), 7.2-7.3 (m, 4H), 7.1-7.2 (m, 2H), 4.41 (s, 1H), 4.0-4.1 (m, 1H), 3.6-3.7 (m, 1H), 3.2-3.1 (m, 2H), 2.8-2.9 (m, 2H), 2.76 (t, 1H, J=6.8 Hz), 2.69 (t, 1H, J=6.8 Hz), 2.5-2.6 (m, 1H), 2.4-2.5 (m, 1H), 1.85 (dd, 1H, J=11.2, 4.0 Hz), 1.6-1.7 (m, 1H), 1.38 (s, 9H), 1.12 (d, 3H, J=6.7 Hz).

Step 3. To a 450 mL mini clave was added tert-butyl (R)-4-(1-benzhydryl)azetidin-3-yl)-2-methylpiperazine-1-carboxylate (8.5 g, 20.16 mmol). MeOH (40 mL), and EtOAc (40 mL). Pd/C (4.29 g, 50 wt %, Hindustan Platinum) was added under N₂ atmosphere and reaction mixture was stirred under H₂ atmosphere (70 psi) for 48 h. The reaction mixture was filtered through a celite bed and washed with MeOH (500 mL). The filtrate was concentrated to give tert-butyl (R)-4-(azetidin-3-yl)-2-methylpiperazine-1-carboxylate. This crude material was used for next step without purification. m/z (ESI): 256.3 (M+H)⁺. ¹H NMR (400 MHz, DMSO-d₆) δ ppm 4.08 (m, 1H), 3.6-3.7 (m, 1H), 3.46 (s, 2H), 2.8-3.1 (m, 4H), 2.6-2.7 (m, 2H), 2.48 (t, 1H, J=1.8 Hz), 1.88 (dd, 1H, J=11.2, 4.0 Hz), 1.75-1.65 (m, 1H), 1.40 (s, 9H), 1.15 (d, 3H, J=6.7 Hz).

The intermediate in the table below was prepared in a fashion similar to that described above for intermediate D-8. Synthesis of the building blocks is either described above or from commercial sources.

| | | | m/z (ESI): |
|---|---|---|---|
| Int. # | Chemical Structure | Name | (M + H)⁺ |
| D-9 | 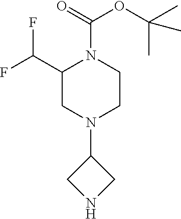 [SMILES: CC(C)(C)OC(=O)N1CCN(C2CNC2)CC1C(F)F] | tert-butyl 4-(azetidin-3-yl)-2- (difluoromethyl)piperazine-1-carboxylate | 292.0 |

### 1-Benzyl-4-(2-(((tert-butyldiphenylsilyl)oxy)methyl)azetidin-3-yl)piperazine (Intermediate D-10)

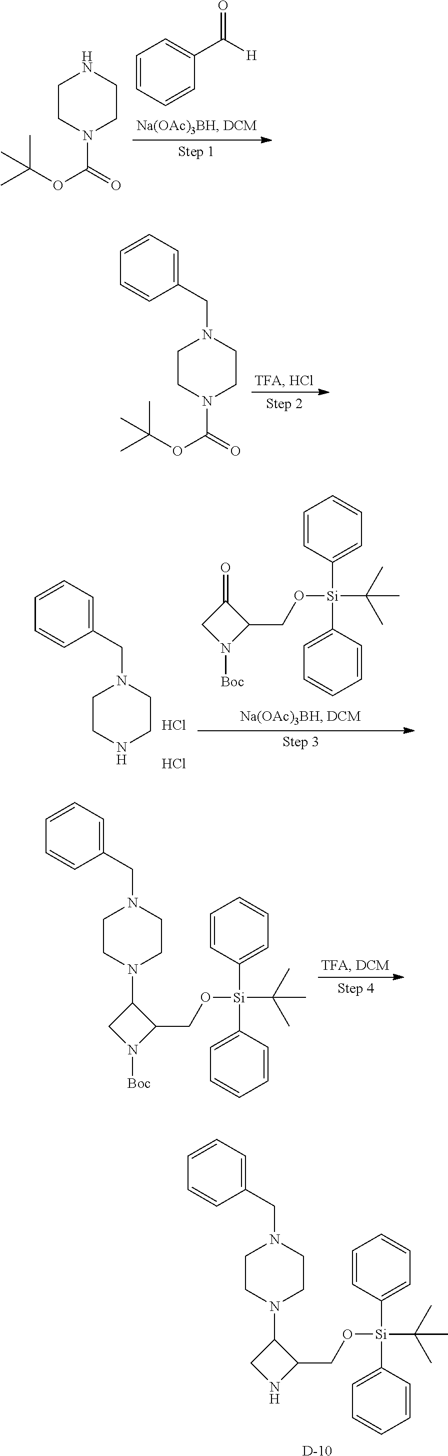

Step 1. Toa 150 mL und-bottomed flask, was added ter-butyl piperazine-1-carboxylate (2.60 g, 13.96 mmol, Combi-Blocks Inc.), benzaldehyde (1.6 mL, 15.36 mmol, Sigma-Aldrich, Inc.). AcOH ((1.2 mL, 3.49 mmol, Sigma-Aldrich, Inc.), and DCM (28 mL). The mixture was cooled to 0° C., then sodium triacetoxyborohydride (8.88 g, 41.9 mmol) was added and the mixture was stirred at 0° C. for 30 min, then at rt for 16 h. The reaction mixture was quenched with sat. aq. NaH COs solution and stirred 30 min. The extracts were separated and the aqueous layer was extracted with DCM (3×). The combined organic extracts were dried over MgSO₄, filtered, and concentrated in vacuo. The crude material was absorbed onto a plug of silica gel and purified by chromatography through a Redi-Sep pre-packed silica gel column, eluting with a gradient of 0% to 35% EtOAc in heptane, to provide tert-butyl 4-benzylpiperazine-1-carboxylate. ¹H NMR (400 MHz, chloroform-d) δ ppm 7.29-7.40 (m, 5H), 3.53 (s, 2H), 3.42-3.49 (m, 4H), 2.36-2.47 (m, 4H), 1.48 (s, 9H) m/z (ESI): 277.3 (M+H)⁺.

Step 2. To a 150 mL round-bottomed flask was added tert-butyl 4-benzylpiperazine-1-carboxylate (2.96 g, 1 (0.71 mmol), TFA (12.2 mL, 107 mmol, Apollo Scientific Ltd.), and DCM (53.5 mL). The reaction mixture was stirred at it for 18 h and then concentrated in vacuo. The residue was diluted with DCM (53.5 mL), then treated with 4.0 M hydrogen chloride solution in dioxane (6.7 mL, 26.8 mmol, Sigma-Aldrich. Inc.). The resulting mixture was stirred for 1.5 h, then concentrated in vacuo. The residue was diluted with heptane and the precipitate was collected by filtration. The product was washed with (1:1) DCM/heptane, then dried in a vacuum oven (40° C.) for 3 h to afford 1-benzylpiperazin-dihydrochloride. ¹H NMR (400 MHz, MeOH-d₄) δ ppm 7.59-7,7-(m, 2H), 7.54 (br d, J=2.4 Hz, 3H), 4.50 (s, 2H), 3.52-3.72 (m, 8H). (Note: —NH proton not observed m/z (ESI): 177.2 (M+H)⁺.

Step 3. To a round-bottomed flask was added 1-benzylpiperazine dihydrochloride (0.57 g, 2.27 mmol) and DCM (5.7 mL). TEA (0.4 mL, 2.502 mmol, Sigma-Aldrich. Inc.) was slowly added and the reaction mixture was stirred for 5 min. tert-butyl 2-(((tert-butyldiphenylsilyl)oxy)methyl)-3-oxoazetidine-1-carboxylate (0.500 g, 1.137 mmol) was slowly added to the reaction and the reaction mixture was stirred at rt for 15 min. Then sodium triacetoxyborohydride (0.723 g, 3.41 mmol, Sigma-Aldrich. Inc.) was slowly added to the reaction, and the reaction mixture was stirred at rt for 3 days. The reaction mixture was quenched with sat. aq. NaHCO₃ and stirred 10 min. The mixture was diluted with DCM and the extracts were separated. The aqueous lay er was extracted with DCM (3×). The combined organic extracts were dried over MgSO₄, filtered, and concentrated in vacuo. The crude material was absorbed onto a plug of silica gel and purified by chromatography through an ISCO 40 gram silica column, eluting with a gradient of 0% to 60% EtOAc in heptane, to afford tert-butyl 3-(4-benzylpiperazin-1-yl)-2-(((tert-butyldiphenylsilyloxy)methyl)azetidine-1-carboxylate. ¹H NMR (400 MHz, chloroform-d) δ ppm 7.71 (br s, 4H), 7.29-7.50 (m, 11H), 4.19-4.39 (m, 2H), 3.82 (br t, J=8.0 Hz, 2H), 3.71 (br s, 1H), 3.53 (br s, 2H), 3.27 (br s, 1H), 2.64 (br s, 2H), 2.46 (br s, 3H), 2.26-2.38 (m, 1H), 1.52-1.60 (m, 2H), 1.33 (br s, 9H), 1.03-1.09 (m, 9H). m/z (ESI): 600.2 (M+H)⁺.

Step 4. To a 100 mL round-bottomed flask was added tert-butyl 3-(4-benzylpiperazin-1-yl)-2-(((tert-butyldiphenylsilyl)oxy)methyl)azetidine-1-carboxylate (0.346 g, 0.577 mmol) and TFA (0.66 mL, 5.77 mmol. Apollo Scientific Ltd.) in DCM (2.9 mL). The reaction mixture was stirred at rt for 16 h. The reaction mixture was diluted with DCM and H₂O. The mixture was neutralized with sat. aq. NaHCO₃ (pH˜6-7) and the extracts were separated. The aqueous layer was extracted with DCM (3×) and the combined organic extracts were dried over MgSO₄, filtered, and concentrated in vacuo. The crude residue was used without further purification. m/z (ESI): 510.3 (M+H)⁺

### Core/Linker

### Tert-Butyl 4-((2R,3R)-1-(6-chloro-2-(trifluormethyl)pyrimidin-4-yl)-2-methyl)azetidin-3-yl)piperazine-1-carboxylate (Intermediate E-1)

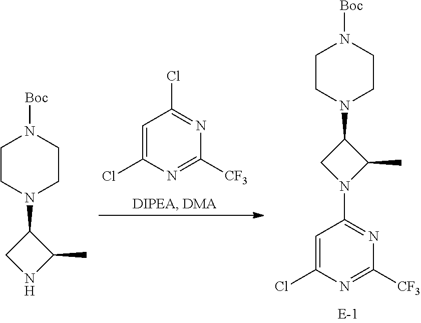 [SMILES: C[C@H]1NC[C@H]1N1CCN(C(=O)OC(C)(C)C)CC1 ; FC(F)(F)c1nc(Cl)cc(Cl)n1 ; C[C@@H]1[C@H](N2CCN(C(=O)OC(C)(C)C)CC2)CN1c1cc(Cl)nc(C(F)(F)F)n1]

To a mixture of tert-butyl 4-((2R,3R)-2-methyl)azetidin-3-yl)piperazine-1-carboxylate (4.5 g, 17.62 mmol), and 4,6-dichloro-2-(trifluoromethyl)pyrimidine (4.21 g, 19.38 mmol, Combi-Blocks, Inc.) in DMA (45 mL) was added DIPEA (9.23 mL, 52.9 mmol, Sonia Industries) and the reaction mixture was stirred at rt for 3 h. The reaction mixture was quenched with H₂O (200 mL) and extracted with EtOAc (100 mL). The organic extract was washed with brine (60 mL)(dried over Na₂SO₄, filtered, and concentrated. The crude residue was purified by chromatography (silica, 15% to 20% EtOAc in hexanes) to give tert-butyl 4-((2R,3R)-1-(6-chloro-2-(trifluoromethyl)pyridin-4-yl-2-methyl)azetidin-3-yl)piperazine-1-carboxylate. m/z (ESI): 436.1 (M+H)⁺. ¹H NMR (400 MHz, DMSO-d₆) δ 6.7-6.9 (m, 1H), 4.61 (q, 1H, J=5.4 Hz), 4.09 (t, 1H, J=8.8 Hz), 3.99 (t, 1H, J=8.5 Hz), 3.3-3.4 (m, 4H), 2.23 (d, 4H, J=8.5 Hz), 1.3-1.5 (m, 12H). ¹⁹F NMR (377 MHz, DMSO-d₆) δ−70.20 (d, J=37.8 Hz).

The intermediates in the table below were prepared in a fashion similar to that was described above for intermediate E-1. Synthesis of the building blocks is either described above or from commercial

| | | | m/z (ESI): |
|---|---|---|---|
| Int. # | Chemical Structure | Name | (M + H)⁺ |
| E-2 | 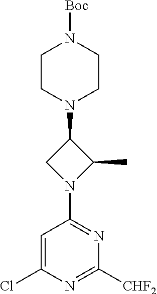 [SMILES: C[C@@H]1[C@H](N2CCN(C(=O)OC(C)(C)C)CC2)CN1c1cc(Cl)nc(C(F)F)n1] | tert-butyl 4-((2R,3R)-1-(6-chloro-2- (difluoromethyl)pyrimidin-4-yl)-2-methylazetidin- 3-yl)piperazine-1-carboxylate | 418.2 |
| E-3 | 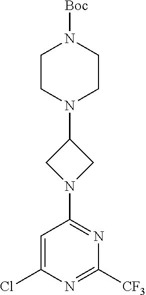 [SMILES: CC(C)(C)OC(=O)N1CCN(C2CN(c3cc(Cl)nc(C(F)(F)F)n3)C2)CC1] | tert-butyl 4-(1-(6-chloro-2- (trifluoromethyl)pyrimidin-4-yl)azetidin-3- yl)piperazine-1-carboxylate | 422.1 |
| E-4 | 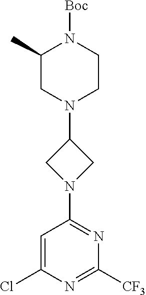 [SMILES: C[C@@H]1CN(C2CN(c3cc(Cl)nc(C(F)(F)F)n3)C2)CCN1C(=O)OC(C)(C)C] | tert-butyl (R)-4-(1-(6-chloro-2- (trifluoromethyl)pyrimidin-4-yl)azetidin-3-yl)-2- methylpiperazine-1-carboxylate | 436.1 |
| E4-1 | 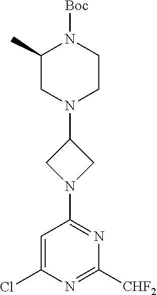 [SMILES: C[C@@H]1CN(C2CN(c3cc(Cl)nc(C(F)F)n3)C2)CCN1C(=O)OC(C)(C)C] | tert-butyl (R)-4-(1-(6-chloro-2- (difluoromethyl)pyrimidin-4-yl)azetidin-3-yl)-2- methylpiperazine-1-carboxylate | 418.7 |
| E-5 | 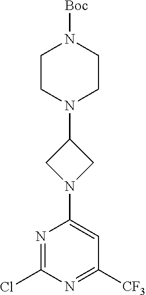 [SMILES: CC(C)(C)OC(=O)N1CCN(C2CN(c3cc(C(F)(F)F)nc(Cl)n3)C2)CC1] | tert-butyl 4-(1-(2-chloro-6- (trifluoromethyl)pyrimidin-4-yl)azetidin-3- yl)piperazine-1-carboxylate | 422.1 |
| E-6 | 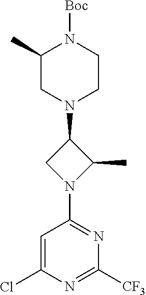 [SMILES: C[C@@H]1CN([C@@H]2CN(c3cc(Cl)nc(C(F)(F)F)n3)[C@@H]2C)CCN1C(=O)OC(C)(C)C] | tert-butyl (R)-4-((2R,3R)-1-(6-chloro-2- (trifluoromethyl)pyrimidin-4-yl)-2-methylazetidin- 3-yl)-2-methylpiperazine-1-carboxylate | 450.6 |
| E-7 | 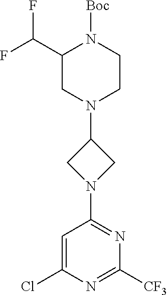 [SMILES: CC(C)(C)OC(=O)N1CCN(C2CN(c3cc(Cl)nc(C(F)(F)F)n3)C2)CC1C(F)F] | tert-butyl 4-(1-(6-chloro-2- (trifluoromethyl)pyrimidin-4-yl)azetidin-3-yl)-2- (difluoromethyl)piperazine-1-carboxylate | 472.1 |
| E-14 | 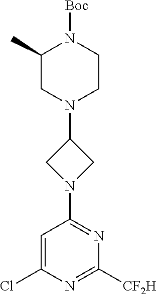 [SMILES: C[C@@H]1CN(C2CN(c3cc(Cl)nc(C(F)F)n3)C2)CCN1C(=O)OC(C)(C)C] | tert-butyl (R)-4-(1-(6-chloro-2- (difluoromethyl)pyrimidin-4-yl)azetidin-3-yl)-2- methylpiperazine-1-carboxylate | 418.0 |
| E-15 | 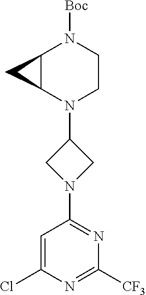 [SMILES: CC(C)(C)OC(=O)N1CCN(C2CN(c3cc(Cl)nc(C(F)(F)F)n3)C2)[C@H]2C[C@H]21] | tert-butyl (1R,6S)-5-(1-(6-chloro-2- (trifluoromethyl)pyrimidin-4-yl)azetidin-3-yl)-2,5- diazabicyclo[4.1.0]heptane-2-carboxylate | 378.1 |
| E-16 | 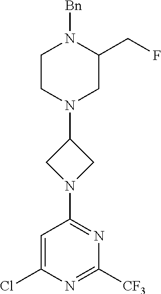 [SMILES: FCC1CN(C2CN(c3cc(Cl)nc(C(F)(F)F)n3)C2)CCN1Cc1ccccc1] | 4-(3-(4-benzyl-3-(fluoromethyl)piperazin-1- yl)azetidin-1-yl)-6-chloro-2- (trifluoromethyl)pyrimidine | 444.1 |
| E-17 | 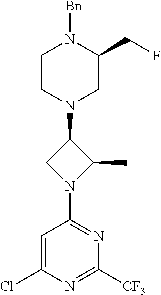 [SMILES: C[C@@H]1[C@H](N2CCN(Cc3ccccc3)[C@@H](CF)C2)CN1c1cc(Cl)nc(C(F)(F)F)n1] | 4-((2R,3R)-3-((R)-4-benzyl-3- (fluoromethyl)piperazin-1-yl)-2-methylazetidin-1- yl)-6-chloro-2-(trifluoromethyl)pyrimidine | 458.1 |
| E-18 | 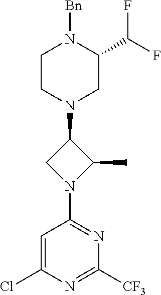 [SMILES: C[C@@H]1[C@H](N2CCN(Cc3ccccc3)[C@H](C(F)F)C2)CN1c1cc(Cl)nc(C(F)(F)F)n1] | 4-((2R,3R)-3-((S)-4-benzyl-3- (difluoromethyl)piperazin-1-yl)-2-methylazetidin- 1-yl)-6-chloro-2-(trifluoromethyl)pyrimidine | 476.3 |
| E-19 | 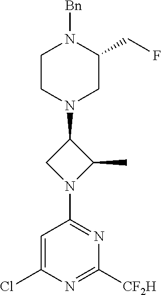 [SMILES: C[C@@H]1[C@H](N2CCN(Cc3ccccc3)[C@H](CF)C2)CN1c1cc(Cl)nc(C(F)F)n1] | 4-((2R,3R)-3-((S)-4-benzyl-3- (fluoromethyl)piperazin-1-yl)-2-methylazetidin-1- yl)-6-chloro-2-(difluoromethyl)pyrimidine | 440.3 |
| E-20 | 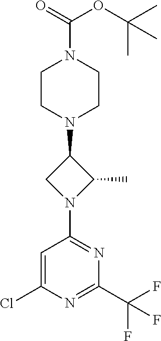 [SMILES: C[C@H]1[C@H](N2CCN(C(=O)OC(C)(C)C)CC2)CN1c1cc(Cl)nc(C(F)(F)F)n1] | tert-butyl 4-((2S,3R)-1-(6-chloro-2- (trifluoromethyl)pyrimidin-4-yl)-2-methylazetidin- 3-yl)piperazine-1-carboxylate | 436.1 |

### Tert-Butyl 4-((2R,3R)-1-(6-fluoro-2-(trifluoromethyl)pyrimidin-4-yl)-2-methyl)azetidin-3-yl)piperazine-1-carboxylate (Intermediate E-8)

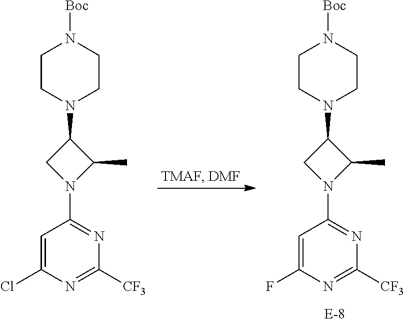 [SMILES: C[C@@H]1[C@H](N2CCN(C(=O)OC(C)(C)C)CC2)CN1c1cc(Cl)nc(C(F)(F)F)n1 ; C[C@@H]1[C@H](N2CCN(C(=O)OC(C)(C)C)CC2)CN1c1cc(F)nc(C(F)(F)F)n1]

tert-Butyl 4-((2R,3R)-1-(6-chloro-2-(trifluoromethyl)pyrimidin-4-yl)-2-methyl)azetidin-3-yl)piperazine-1-carboxylate (26 g, 59.6 mmol) was dissolved in DMF (520 mL). Tetramethylammonium fluoride (13.88 g, 149 mmol, Oakwood) was added under nitrogen atmosphere and the reaction mixture was stirred for 6 days at 60° C. The reaction mixture was cooled to rt and quenched with ice cold water (3 L) and subjected to workup and purification to give tert-butyl 4-((2R,3R)-1-(6-fluoro-2-(trifluoromethyl)pyrimidin-4-yl)-2-methyl)azetidin-3-yl)piperazine-1-carboxylate. m/z (ESI): 420.1 (M+H)⁺. ¹H NMR (400 MHz, DMSO-d₆) δ ppm 6.38 (s, 1H), 4.62-4.55 (m, 1H), 3.9-4.1 (m, 2H), 3.35 (s, 4H), 3.2-3.3 (m, 1H) 2.24 (s, 4H), 1.3-1.4 (m, 12H) ¹⁹F NMR (377 MHz, DMSO-d₆) δ−67.05 (d, 197.4 Hz, 1F), −70.38 (s, 3F).

The intermediates in the table below were prepared in a fashion similar to that described above for intermediate E-8. Synthesis of the building blocks is either described above or from commercial sources.

| | | | m/z (ESI): |
|---|---|---|---|
| Int. # | Chemical Structure | Name | (M + H)⁺ |
| E-9 | 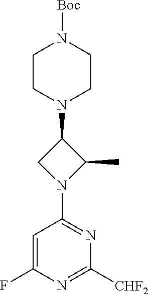 [SMILES: C[C@@H]1[C@H](N2CCN(C(=O)OC(C)(C)C)CC2)CN1c1cc(F)nc(C(F)F)n1] | tert-butyl 4-((2R,3R)-1-(2-(difluoromethyl)-6- fluoropyrimidin-4-yl)-2-methylazetidin-3- yl)piperazine-1-carboxylate | 402.3 |
| E-10 | 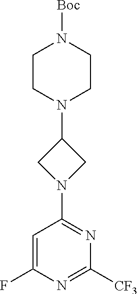 [SMILES: CC(C)(C)OC(=O)N1CCN(C2CN(c3cc(F)nc(C(F)(F)F)n3)C2)CC1] | tert-butyl 4-(1-(6-fluoro-2- (trifluoromethyl)pyrimidin-4-yl)azetidin-3- yl)piperazine-1-carboxylate | 427.9 |
| E-11 | 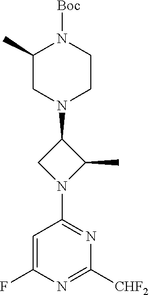 [SMILES: C[C@@H]1CN([C@@H]2CN(c3cc(F)nc(C(F)F)n3)[C@@H]2C)CCN1C(=O)OC(C)(C)C] | tert-butyl (R)-4-((2R,3R)-1-(2-(difluoromethyl)- 6-fluoropyrimidin-4-yl)-2-methylazetidin-3-yl)- 2-methylpiperazine-1-carboxylate | 415.8 |

### Tert-Butyl 4-((2R)-1-(6-chloro-2-(difluoromethyl)pyrimidin-4-yl)-2-methyl)azetidin-3-yl)-2-ethynylpiperazine-1-carboxylate (Intermediate E-12)

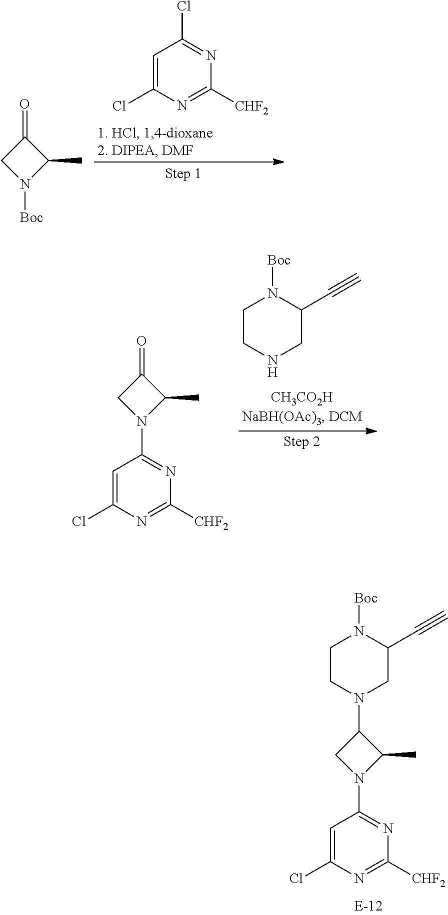 [SMILES: C[C@@H]1C(=O)CN1C(=O)OC(C)(C)C ; FC(F)c1nc(Cl)cc(Cl)n1 ; C[C@@H]1C(=O)CN1c1cc(Cl)nc(C(F)F)n1 ; C#CC1CNCCN1C(=O)OC(C)(C)C ; C#CC1CN(C2CN(c3cc(Cl)nc(C(F)F)n3)[C@@H]2C)CCN1C(=O)OC(C)(C)C]

Step 1. To a solution of 4,6-dichloro-2-(difluoromethyl)pyrimidine (1.074 g, 5.40 mmol, Ambeed, Inc.) in 1,4-dioxane (2 mL) was added hydrogen chloride, (4N in 1,4-dioxane, 5.40 mL, 21.60 mmol) and the reaction mixture was stirred at rt for 1 h. The reaction mixture was concentrated in vacuo to give the crude (R)-2-methyl)azetidin-3-one. The crude material was redissolved in DMF (2 mL) and to the reaction mixture was added 4,6-dichloro-2-(difluoromethyl)pyrimidine (1.074 g, 5.40 mmol, Ambeed, Inc.) and DIPEA (0.943 mL, 5.40 mmol) and the reaction mixture was stirred at rt for 18 h. The reaction mixture was diluted with brine and EtOAc and the organic layer was separated. The aqueous layer was extracted with EtOAc and the combined organic extracts were washed with brine, dried over Na₂SO₄, filtered, and concentrated in vacuo. The crude material was purified by chromatography on silica gel, eluting with a gradient of 0% to 20% EtOAc in heptane, to provide (R)-1-(6-chloro-2-(difluoromethyl)pyrimidin-4-yl)-2-methyl)azetidin-3-one. m/z (ESI): 248.1 (M+H)⁺.

Step 2. To a solution of (R)-1-(6-chloro-2-(difluoroethyl)pyrimidin-4-yl)-2-methyl)azetidin-3-one (1.14 g, 4.60 mmol) and tert-butyl 2-ethynylpiperazine-1-carboxylate (0.968 g, 4.60 mmol, Advanced ChemBlocks Inc.) in DCM (12 mL) was added acetic acid (5 μL, 0.092 mmol, Sigma-Aldrich, Inc.) and the reaction mixture was stirred at rt for 15 min. To the reaction mixture was added sodium triacetoxyborohydride (2.93 g, 13.81 mmol, Sigma-Aldrich, Inc.) in 3 portions and the reaction mixture was stirred at rt for 16 h. H₂O was added to the mixture and stirred at rt for 15 min to quench the reaction. The organic layer was separated, and the aqueous layer was extracted with DCM. The organic extracts were washed with brine, dried over Na₂SO₄, filtered, and concentrated in vacuo. The crude material was purified by chromatography on silica gel, eluting with a gradient of 0% to 20% EtOAc in heptane, to provide tert-butyl 4-((2R)-1-(6-chloro-2-(difluoromethyl)pyrimidin-4-yl)-2-methyl)azetidin-3-yl)-2-ethynylpiperazine-1-carboxylate, which included a mixture of 3 isomers and other impurities. m/z (ESI): 442.0 (M+H)⁺.

### Tert-Butyl (R)-4-(3-(6-chloro-2-(trifluoromethyl)pyrimidin-4-yl)cyclobutyl)-3-(hydroxymethyl)piperazine-1-carboxylate (Intermediate E-13)

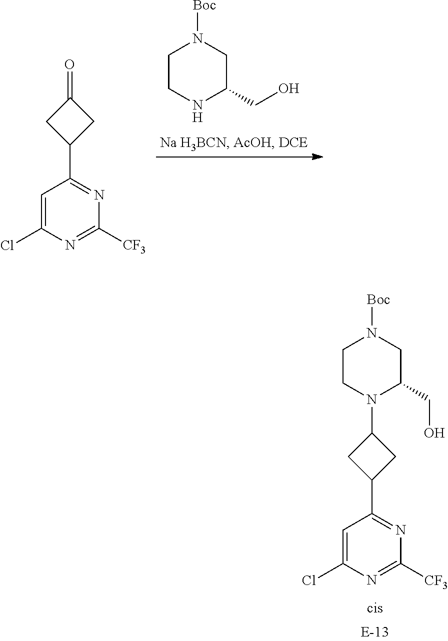 [SMILES: O=C1CC(c2cc(Cl)nc(C(F)(F)F)n2)C1 ; CC(C)(C)OC(=O)N1CCN[C@@H](CO)C1 ; CC(C)(C)OC(=O)N1CCN(C2CC(c3cc(Cl)nc(C(F)(F)F)n3)C2)[C@@H](CO)C1]

A mixture of 3-(6-chloro-2-(trifluoromethyl)pyrimidin-4-yl)cyclobutan-1-one (1.23 g, 4.91 mmol), tert-butyl (R)-3-(hydroxymethyl)piperazine-1-carboxylate (1.062 g, 4.91 mmol) and acetic acid (0.028 mL, 0.491 mmol, Sigma-Aldrich, Inc.) in DCE (30 mL) was stirred at rt for 2 h. Then sodium cyanoborohydride (0.46 g, 7.36 mmol, Sigma-Aldrich Corporation) was added and stirred at rt for 48 h. The reaction was diluted with H₂O and extracted with DCM. The combined organics were dried over Na₂SO₄, filtered. concentrated and chromatographed on silica gel using 0-100% EtOAc in heptane to afford cis-tert-butyl (R)-4-(3-(6-chloro-2-(trifluoromethyl)pyrimidin-4-yl)cyclobutyl)-3-(hydroxymethyl)piperazine-1-carboxylate. m/z (ESI): 451.0 (M+H)⁺. ¹H NMR (500 MHz, DMSO-d₆) δ ppm 7.97 (s, 1H), 4.51 (t, J=5.1 Hz, 1H), 3.56 (dt, J=10.6, 4.4 Hz, 1H), 3.34-3.46 (m, 3H), 3.19-3.28 (m, 2H), 2.61 (br d, J=6.4 Hz, 1H), 2.35-2.49 (m, 3H), 2.14 (dt, J=9.7, 5.0 Hz, 3H), 1.40 (s, 11H).

### 4-Chloro-6-(4-(1,4-dimethyl-1H-pyrazol-5-yl)piperidin-1-yl)-2-(trifluoromethyl)pyrimidine (Intermediate F-1)

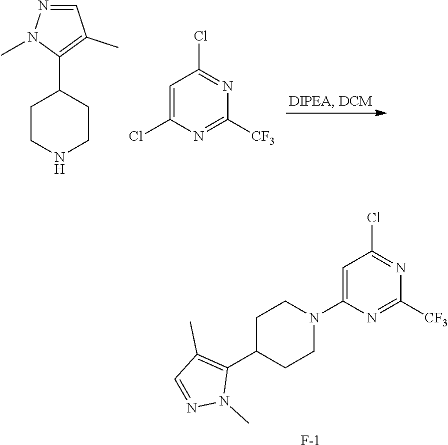

A mixture of 4,6-dichloro-2-trifluoromethylpyrimidine (600 mg, 2.77 mmol. Synchem Inc.) and 4-(1,4-dimethyl-1H-pyrazol-5-yl)piperidine hydrochloride (597 mg, 2.77 mmol) in DCM (5 mL) was stirred at rt for 30 min. Then. DIPEA (2.42 mL, 13.83 mmol) was added and the reaction mixture was stirred at rt for 18 h. Then, the reaction was quenched with H₂O and the organic layer was washed with brine, dried over anhydrous MgSO₄, filtered, and concentrated. The crude material was absorbed onto a plug of silica gel and purified by chromatography using a Biotage Isolera, eluting with a gradient of 0% to 100% (3-1) EtOAc/EtOH in heptane to provide 4-chloro-6-(4-(1,4-dimethyl-1H-pyrazol-5-yl)piperidin-1-yl)-2-(trifluoromethyl)pyrimidine. m/z (ESI): 360.0 (M+H)⁺. ¹H NMR (400 MHz, chloroform-d) δ ppm 7.22 (s, 1H), 6.67 (s, 1H), 4.40-4.77 (m, 2H), 3.87 (s, 3H), 2.98-3.16 (m, 3H), 2.08 (s, 3H), 1.90-2.04 (m, 4H).

The intermediates in the table below were prepared in a fashion similar to that described above for intermediate F-1. Synthesis of the building blocks is either described above or from commercial sources.

| | | | m/z (ESI): |
|---|---|---|---|
| Int. # | Chemical Structure | Name | (M + H)⁺ |
| F-2 | 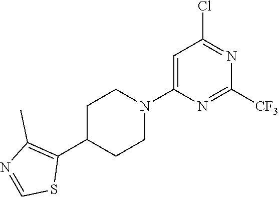 [SMILES: Cc1ncsc1C1CCN(c2cc(Cl)nc(C(F)(F)F)n2)CC1] | 5-(1-(6-chloro-2-(trifluoromethyl)pyrimidin-4- yl)piperidin-4-yl)-4-methylthiazole | 363.1 |
| F-3 | 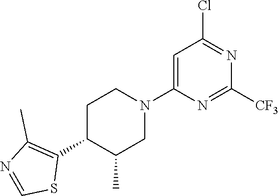 [SMILES: Cc1ncsc1[C@@H]1CCN(c2cc(Cl)nc(C(F)(F)F)n2)C[C@@H]1C] | 5-((3R*,4R*)-1-(6-chloro-2- (trifluoromethyl)pyrimidin-4-yl)-3- methylpiperidin-4-yl)-4-methylthiazole | 377.1 |
| F-4 | 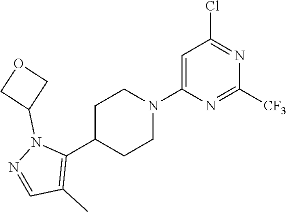 [SMILES: Cc1cnn(C2COC2)c1C1CCN(c2cc(Cl)nc(C(F)(F)F)n2)CC1] | 4-chloro-6-(4-(4-methyl-1-(oxetan-3-yl)-1H- pyrazol-5-yl)piperidin-1-yl)-2- (trifluoromethyl)pyrimidine | 402.1 |
| F-5 | 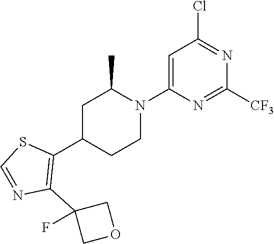 [SMILES: C[C@@H]1CC(c2scnc2C2(F)COC2)CCN1c1cc(Cl)nc(C(F)(F)F)n1] | 5-((2R)-1-(6-chloro-2- (trifluoromethyl)pyrimidin-4-yl)-2- methylpiperidin-4-yl)-4-(3-fluorooxetan-3- yl)thiazole | 437.1 |

### 3-(6-((3S,4R)-4-(1-(2-Methoxyethyl)-4-methyl-1H-pyrazol-5-yl)-3-methylpiperidin-1-yl)-2-(trifluoromethyl)pyrimidin-4-yl)-2,2-dimethylcyclobutane-1-one (Intermediate F-6)

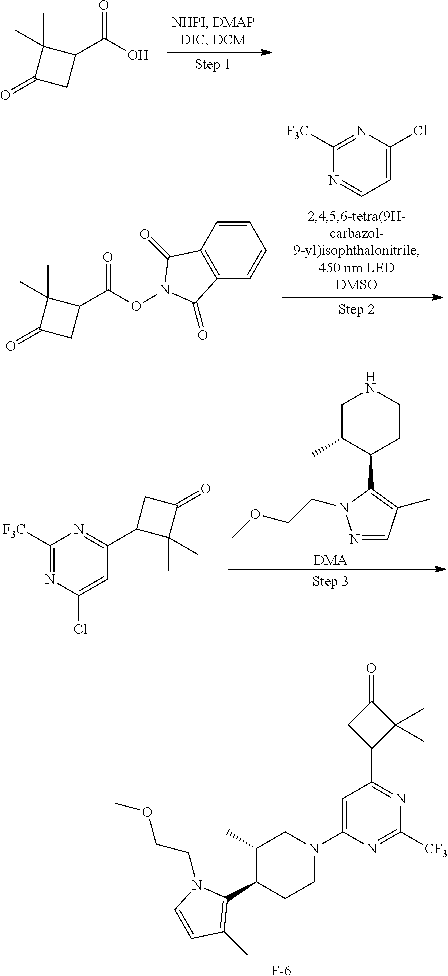

Step 1. To a glass vial was added 2-hydroxyisoindoline-1,3-dione (126 mg, 0.774 mmol, Sigma-Aldrich, Inc.) and 2,2-dimethyl-3-oxocyclobutane-1-carboxylic acid (100 mg, 0.703 mmol, Enamine), and DCM (7 mL). To the mixture was then added (E)-N-((isopropylanilino)methideylene)propan-2-aminium (131 μL, 0.844 nmol, Sigma-Aldrich, Inc.) and the reaction was stirred at rt for 18 h. The reaction was filtered and washed with EtOAc and the filtrate was concentrated. The crude material was absorbed onto a plug of silica gel and purified by chromatography, eluting with a gradient of 0% to 100% EtOAc in heptane, to provide 1,3-dioxoisoindolin-2-yl 2,2-dimethyl-3-oxocyclobutane-1-carboxylate. m/z (ESI): 310.0 (M+Na)⁺.

Step 2. To a glass vial was added 4-chloro-2-(trifluoromethyl)-1,3-diazine (241 mg, 1.323 mmol, Combi-Blocks. Inc.) and 1,3-dioxoisoindolin-2-yl 2,2-dimethyl-3-oxocyclobutane-1-carboxylate (190 mg, 0.661 mmol) in DMSO (3307 μL). The solution was degassed with Ar for 5 min, and then 2,4,5,6-tetra(9-carbazolyl) isophthalonitrile (5.22 mg, 6.61 μmol, eNovation Chemicals LLC) was added to the mixture, and the reaction was irradiated at 450 nm (LED power 100%) for 23 h. The crude material was absorbed onto a plug of silica gel and purified by chromatography, eluting with a gradient of 01% to 60% EtOAc in heptane, to provide 3-(6-chloro-2-(trifluoromethyl)pyrimidin-4-yl)-2,2-dimethylcyclobutane-1-one, that was used as is in the next step. m/z (ESI): 279.0 (M+H)⁺.

Step 3. To a vessel was added 3-(6-chloro-2-(trifluoromethyl)pyrimidin-4-yl)-2,2-dimethylcyclobutane-1-one (46 mg, 0.165 mmol), (3S,4R)-4-(1-(2-methoxyethyl)-4-methyl-1H-pyrazol-5-yl)-3-methylpiperidine (39.2 mg, 0.165 mmol) and DMA (1651 μL). To the mixture was then added DIPEA (86 μL, 0.495 mmol) and the reaction was stirred at 50° C. The reaction was diluted with brine (10 mL), extracted with EtOAc (3×10 mL), dried over MgSO₄, filtered, and concentrated. The crude material was absorbed onto a plug of silica gel and purified by chromatography, eluting with a gradient of 0% to 100% DCM with 10% MeOH, in DCM, to provide 3-(6-((3S,4R)-4-(l-(2-methoxyethyl)-4-methyl-1H-pyrazol-5-yl)-3-methylpiperidin-1-yl)-2-(trifluoromethyl)pyrimidin-4-yl)-2,2-dimethylcyclobutane-1-one. m/z (ESI): 480.0 (M+H)⁺.

The intermediate in the table below was prepared in a fashion similar to that described above for intermediate F-6. Synthesis of the building blocks is either described above or from commercial sources.

| | | | m/z (ESI): |
|---|---|---|---|
| Int. # | Chemical Structure | Name | (M + H)⁺ |
| F-7 | 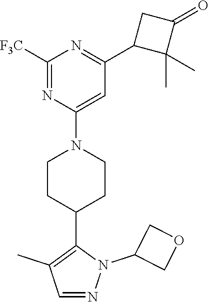 [SMILES: Cc1cnn(C2COC2)c1C1CCN(c2cc(C3CC(=O)C3(C)C)nc(C(F)(F)F)n2)CC1] | 2,2-dimethyl-3-(6-(4-(4-methyl-1-(oxetan-3- yl)-1H-pyrazol-5-yl)piperidin-1-yl)-2- (trifluoromethyl)pyrimidin-4-yl)cyclobutan-1- one | 464.1 |

### 2-Methyl-1-(6-(4-(thiazol-2-yl)piperidin-1-yl)-2-(trifluoromethyl)pyrimidin-4-yl)azetidin-3-one (Intermediate G-1

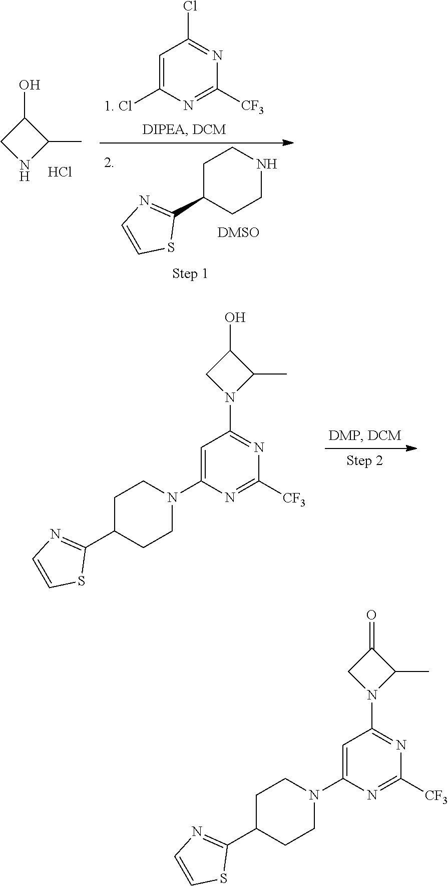

Step 1. To a mixture of 3-hydroxy-2-methyl)azetidin-1-ium chloride (295 mg, 2.39 mmol) and 4,6-dichloro-2-(trifluoromethyl)pyrimidine (432 mg, 1.99 mmol) in DCM (7 mL)/THF (5 mL) was added DIPEA (1.043 mL, 5.97 mmol, Acros) and the mixture was stirred at rt for 3 h. The solvent was evaporated at the residue was redissolved in DMSO (5 mL) and added 2-(piperidin-4-yl)thiazole (335 mg, 1.99 mmol) followed by DIPEA (1.043 mL, 5.97 mmol, Acros). The mixture was stirred at 100° C. for 16 h and cooled to rt. The reaction mixture was diluted with brine and EtOAc and the organic layer was separated. The aqueous layer was extracted with EtOAc and the combined organic extracts were washed with brine, dried over Na₂SO₄, filtered, and concentrated in vacuo. The crude material was purified by chromatography on silica gel, eluting with a gradient of 5% to 100% EtOAc in heptane, to provide 2-methyl-1-(6-(4-(thiazol-2-yl)piperidin-1-yl)-2-(trifluoromethyl)pyrimidin-4-yl)azetidin-3-ol. m/z (ESI): 400.4 (M+H)⁺.

Step 2. To a solution of 2-methyl-1-(6-(4-(thiazol-2-yl)piperidin-1-yl)-2-(trifluoromethyl)pyrimidin-4-yl)azetidin-3-ol (180 mg, 0.451 mmol) In DCM (10 mL) was added dess-martinperiodinane (220 mg, 0.519 mmol). The reaction mixture was stirred at rt for 3 h and directly purified by silica gel chromatography eluting with a gradient of 0% to 100% EtOAc in heptane, to provide 2-methyl-1-(6-(4-(thiazol-2-yl)piperidin-1-yl)-2-(trifluoromethyl)pyrimidin-4-yl)azetidin-3-one. m/z (ESI): 398 (M+H)⁺. ¹H NMR (300 MHz, CHLOROFORM-d) δ ppm 7.76 (d, J=3.39 Hz, 1H) 7.29-7.29 (m, 1H) 7.30 (br s, 1H) 5.51 (s, 1H) 5.10 (ddd, J=6.92, 3.44, 1.32 Hz, 1H) 4.76-4.79 (m, 1H) 4.50 (br d, J=11.68 Hz, 2H) 3.40 (br d, J=3.58 Hz, 1H) 3.12 (br t, J=12.72 Hz, 2H) 2.27 (br d, J=10.93 Hz, 2H) 1.81-1.92 (m, 2H) 1.61 (d, J=6.97 Hz, 3H).

The intermediate in the table below was prepared in a fashion similar to that described above for intermediate G-1. Synthesis of the building blocks is either described above or from commercial sources.

| | | | m/z (ESI): |
|---|---|---|---|
| Int. # | Chemical Structure | Name | (M + H)⁺ |
| G-2 | 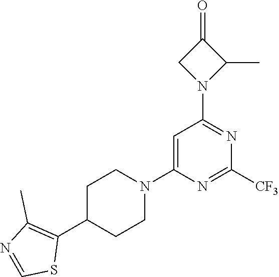 [SMILES: Cc1ncsc1C1CCN(c2cc(N3CC(=O)C3C)nc(C(F)(F)F)n2)CC1] | 2-methyl-1-(6-(4-(4-methylthiazol-5- yl)piperidin-1-yl)-2-(trifluoromethyl)pyrimidin- 4-yl)azetidin-3-one | 412.2 |

### Section 2: Synthesis of Example Compounds

### Example 2-001: 1-(4-(1-(6-(4-(4-chloro-1-methyl-1H-pyrazol-5-yl)piperidin-1-yl)-2-(trifluoromethyl)pyrimidin-4-yl)azetidin-3-yl)piperazin-1-yl)prop-2-en-1-one

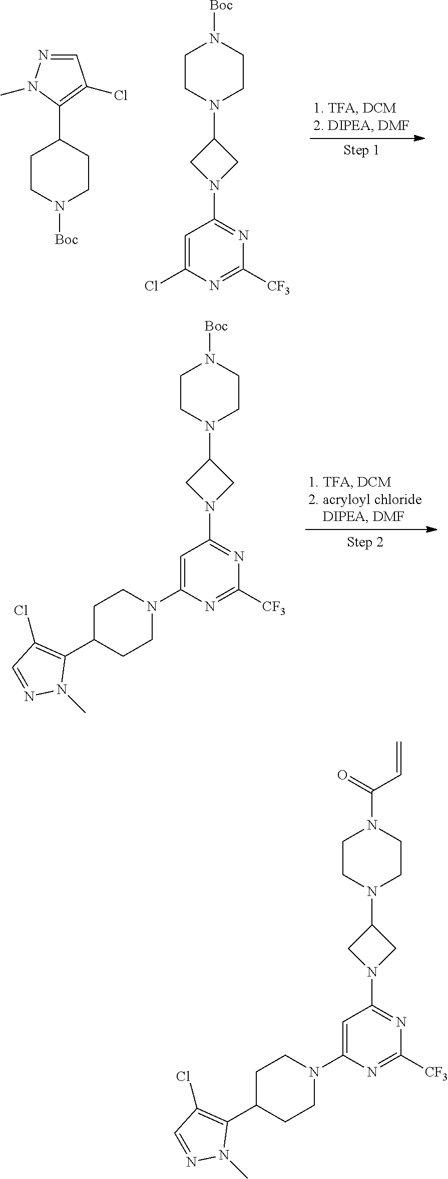

Step 1. To a solution of ter-butyl 4-(4-chloro-1-methyl-1H-pyrazol-5-yl)piperidine-1-carboxylate (150 mg, 0.50 mmol) in DCM (1 mL) was added TFA (0.5 mL, 6.71 mmol, Sigma-Aldrich. Inc.), and the reaction mixture was stirred at rt for 30 min. The reaction mixture was concentrated in vacuo to give the crude 4-(4-chloro-1-methyl-1H-pyrazol-5-yl)piperidine which was directly used in the next step. m/z (ESI): 200.2 (M+H)⁺. To the reaction vessel containing crude 4-(4-chloro-1-methyl-1H-pyrazol-5-yl)piperidine was added tert-butyl 4-(1-(6-chloro-2-(trifluoromethyl)pyrimidin-4-yl)azetidin-3-yl)piperazine-1-carboxylate (211 mg, 0.50 mmol), DIPEA (0.88 mL, 5.00 mmol, Sigma-Aldrich. Inc.), and DMF (2 mL). The reaction mixture was heated to 100° C., and stirred for 6 h. The reaction mixture was diluted with brine and extracted with EtOAc (2×). The combined organic extracts were dried over a plug of anhydrous Na₂SO₄ and concentrated in vacuo. The crude material was purified by chromatography through a Redi-Sep pre-packed silica gel column, eluting with a gradient of 0% to 60% (3:1) EtOAc/EtOH in heptane, to provide tert-butyl 4-(1-(6-(4-(4-chloro-1-methyl-1H-pyrazol-5-yl)piperidin-1-yl)-2-(trifluoromethyl)pyrimidin-4-yl)azetidin-3-yl)piperazine-1-carboxylate. m/z (ESI): 585.0 (M+H)⁺.

Step 2. To a solution of tert-butyl 4-(1-(6-(4-(4-chloro-1-methyl-1H-pyrazol-5-yl)piperidin-1-yl)-2-(trifluoromethyl)pyrimidin-4-yl)azetidin-3-yl)piperazine-1-carboxylate (0.127 g, 0.218 mmol) in DCM (2 mL) was added TFA (0.5 mL, 0.218 mmol), and the reaction mixture was stirred at rt for 30 min. The reaction mixture was concentrated in vacuo to give crude 4-(4-(4-chloro-1-methyl-1H-pyrazol-5-yl)piperidin-1-yl)-6-(3-(piperazin-1-yl)azetidin-1-yl)-2 (trifluoromethyl)pyrimidine that was directly used in the next step. m/z (ESI): 484.9 (M+H)⁺. 4-(4-(4-Chloro-1-methyl-1H-pyrazol-5-yl)piperidin-1-yl)-6-(3-(piperazin-1-yl)azetidin-1-yl)-2 (trifluoromethyl)pyrimidine was added to DCM (2 mL), DIPEA (0.190 mL, 1.09 mmol, Sigma-Aldrich, Inc.) and a 0.2 M solution of acryloyl chloride (1.09 mL, 0.218 mmol, Sigma-Aldrich, Inc.) in DCM. The reaction mixture was stirred at rt for 30 min. The reaction mixture was diluted with said. aq. NH₄Cl and extracted with DCM (3×). The organic extracts were dried over anhydrous Na₂SO₄ and concentrated in vacuo. The crude material was purified by chromatography through a Redi-Sep pre-packed silica gel column, eluting with a gradient of 0% to 100% (3:1) EtOAc/EtOH in heptane, to provide 1-(4-(1-(6-(4-(4-chloro-1-methyl-1H-pyrazol-5-yl)piperidin-1-yl)-2-(trifluoromethyl)pyrimidin-4-yl)azetidin-3-yl)piperazin-1-yl)prop-2-en-1-one. m/z (ESI): 539.0 (M+H)⁺. ¹H NMR (400 MHz. chloroform-d) δ ppm 7.31-7.36 (m, 1H), 6.50-6.60 (m, 1H), 6.25-6.34 (m, 1H), 5.71 (dd, J=10.7, 1.9 Hz, 1H), 5.29-5.33 (m, 1H), 4.50-4.60 (m, 2H), 4.08-4.18 (m, 2H), 3.91-3.98 (m, 2H), 3.86 (s, 3H), 3.57-3.78 (m, 4H), 3.28-3.41 (m, 1H), 2.97-3.0) (m, 1H), 2.90 (td, J=12.9, 2.4 Hz, 2H), 2.43 (t, J=5.0 Hz, 4H), 2.08-2.28 (m, 2H), 1.79-1.86 (m, 2H) ¹⁹F NMR (376 MHz, chloroform-d) δ ppm −71.67 (s, 3F).

### Example 2-002: 1-(4-((2R,3R)-2-Methyl-1-(6-(4-(4-methyl-1-(oxetan-3-yl)-1H-pyrazol-5-yl)piperidin-1-yl)-2-(trifluoromethyl)pyrindin-4-yl)azetidin-3-yl)piperazin-1-yl)prop-2-en-1-one

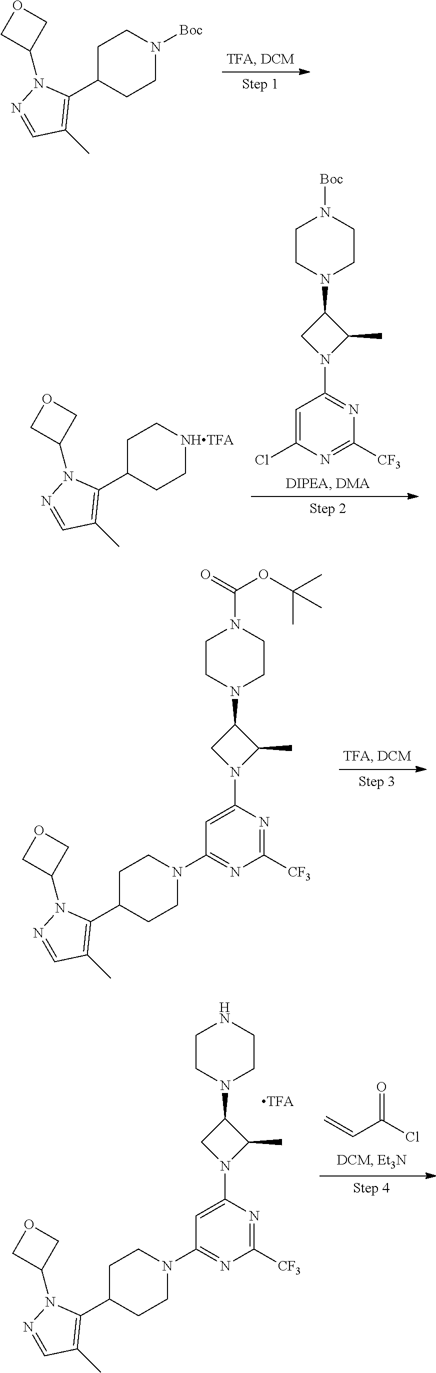

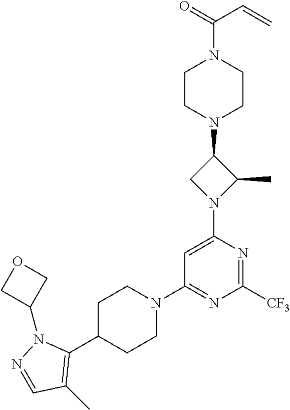 [SMILES: C=CC(=O)N1CCN([C@@H]2CN(c3cc(N4CCC(c5c(C)cnn5C5COC5)CC4)nc(C(F)(F)F)n3)[C@@H]2C)CC1]

Step 1. Tert-butyl 4-(4-methyl-1-(oxetan-3-yl)-1H-pyrazol-5-yl)piperidine-1-carboxylate (33 g, 103 mmol) was dissolved in DCM (660 mL) at 0° C. TFA (150 mL, 103 mmol, Symax Laboratories) was added dropwise at 0° C., and the reaction mixture was stirred at rt for 2 h. The reaction mixture was concentrated under reduced pressure and co-evaporated with toluene (250 mL) under reduced pressure to give 4-(4-methyl-1-(oxetan-3-yl)-1H-pyrazol-5-yl)piperidine trifluoroacetate salt. The crude residue was used in the next step without purification. m/z (ESI): 221.1 (M+H)⁺.

Step 2. 4-(4-Methyl-1-(oxetan-3-yl)-1H-pyrazol-5-yl)piperidine trifluoroacetate salt (30 g, 94 mmol) was dissolved in DMA (300 mL) and tert-butyl 4-((2R,3R)-1-(6-chloro-2-(trifluoromethyl)pyrimidin-4-yl)-2-methyl)azetidin-3-yl)piperazine-1-carboxylate (30 g, 68.8 mmol) and DIPEA (120 mL, 688 mmol, Sonia Industries) were added and the reaction mixture was stirred at 90° C. for 16 h. The reaction mixture was cooled, quenched with ice water (1000 mL), and extracted with EtOAc (1000 mL). The organic extract was dried over Na₂SO₄, filtered, and concentrated. The crude residue was purified by chromatography (silica, 0 to 80% EtOAc:hexanes) to give tert-butyl 4-((2R,3R)-2-methyl-1-(6-(4-4-methyl-1-(oxetan-3-yl)-1H-pyrazol-5-yl)piperidin-1-yl)-2-(trifluoromethyl)pyrimidin-4-yl)azetidin-3-yl)piperazine-1-carboxylate. m/z (ESI): 621.2 (M+H)⁺. ¹H NMR (400 MHz, DMSO-d₆) δ ppm 7.30 (s, 1H), 5.81 (s, 1H), 5.6-5.7 (m, 2H), 4.93 (t, 2H, J=6.2 Hz), 4.87 (dd, 2H, J=7.7, 6.0 Hz), 4.47 (q, 3H, J=11.2, 9.0 Hz), 3.7-4.1 (m, 2H), 3.23 (q, 4H, J=7.2 Hz), 3.02 (td, 1H, J=10.8, 4.8 Hz), 2.91 (t, 2H, J=12.3 Hz), 2.23 (d, 4H, J=5.3 Hz), 1.99 (d, 3H, J=1.7 Hz), 1.6-1.8 (m, 4H), 1.39 (d, 12H, J=7.7 Hz).

Step 3. To a solution of ter-butyl 4-((2R,3R)-2-methyl-1-(6-(4-(4-methyl-1-(oxetan-3-yl)-1H-pyrazol-5-yl)piperidin-1-yl)-2-(trifluoromethyl)pyrimidin-4-yl)azetidin-3-yl)piperazine-1-carboxylate (34 g, 54.8 mmol) in DCM (400 mL) at 0° C. was added TFA (200 mL, 2596 mmol, Symax Laboratories), and the reaction mixture was stirred at rt for 1 h. The reaction was concentrated under reduced pressure and triturated with diethyl ether (500 mL) to give 2,2,2-trifluoroacetaldehyde compound with 4-(4-(4-methyl-1-(oxetan-3-yl)-1H-pyrazol-5-yl)piperidin-1-yl)-6-((2R,3R)-2-methyl-3-(piperazin-1-yl)azetidin-1-yl)-2-(trifluoromethyl)pyrimidin trifluoroacetate salt. The crude residue was used in the next step without further purification. m/z (ESI): 521.2 (M+H)⁺.

Step 4. 4-(4-(4-Methyl-1-(oxetan-3-yl)-1H-pyrazol-5-yl)piperidin-1-yl)-6-((2R,3R)-2-methyl-3-(piperazin-1-yl)azetidin-1-yl)-2-(trifluoromethyl)pyrimidine trifluoroacetate salt (10 g, 16.16 mmol) was dissolved in DCM (100 mL) under nitrogen atmosphere. The reaction mixture was cooled to −78° C. TEA (11.27 mL, 81 mmol, Sonia Industries) was added dropwise over 5 min followed by a solution of acryloyl chloride (1.31 mL, 16.16 mmol, Symax Laboratories) in DCM (5 mL) and stirred for 15 min. The reaction mixture was quenched with ice water (300 mL), extracted with EtOAc (500 mL). The organic extract was washed with brine (200 mL), dried over Na₂SO₄, filtered, and concentrated. The crude material was purified by reverse phase HPLC eluting with 40% to 50% MeCN in H₂O to give 1-(4-((2R,3R)-2-methyl-1-(6-(4-(4-methyl-1-(oxetan-3-yl)-1H-pyrazol-5-yl)piperidin-1-yl)-2-(trifluoromethyl)pyrimidin-4-yl)azetidin-3-yl)piperazin-1-yl)prop-2-en-1-one. m/z (ESI): 575.3 (M+H)⁺. ¹H NMR (400 MHz, DMSO-d₆) δ ppm 7.30 (s, 1H), 6.80 (dd, 1H, J=16.7, 10.5 Hz), 6.11 (dd, 11H, J=16.7, 2.4 Hz), 5.5-5.7 (m, 3H), 4.8-5.1 (m, 4H), 4.3-4.6 (m, 3H), 3.95 (t, 1H, J=8.4 Hz), 3.87 (t, 1H, J=7.8 Hz), 3.59 (d, 4H, J=7.5 Hz), 3.25 (q, 1H, J=7.2 Hz), 3.03 (dd, 1H, J=10.8, 5.3 Hz), 2.91 (t, 2H, J=12.0 Hz), 2.29 (s, 4H), 1.99 (s, 3H), 1.6-1.8 (m, 4H), 1.40 (d, 3H, J=6.3 Hz). ¹⁹F NMR (377 MHz, DMSO-d₆) δ−70.22 (d, 1F).

### Example 2-003: 1-(4-((2R,3R)-1-(6-(4-(1-(2-Methoxyethyl)-4-methyl-1H-pyrazol-5-yl)piperidin-1-yl)-2-(trifluoromethyl)pyrimidin-4-yl)-2-methyl)azetidin-3-yl)piperazin-1-yl)prop-2-en-1-one

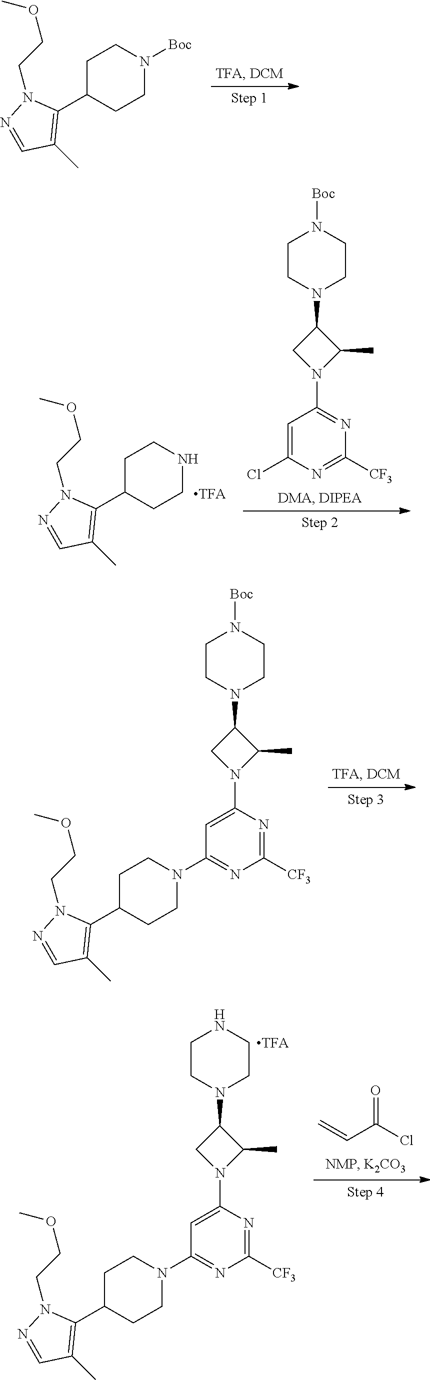

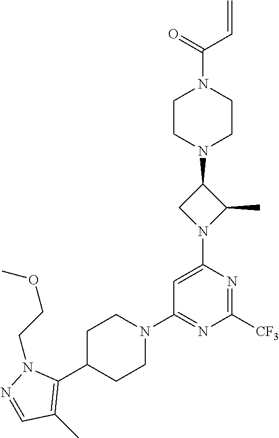 [SMILES: C=CC(=O)N1CCN([C@@H]2CN(c3cc(N4CCC(c5c(C)cnn5CCOC)CC4)nc(C(F)(F)F)n3)[C@@H]2C)CC1]

Step 1. Tert-butyl 4-(1-(2-methoxyethyl)-4-methyl-1H-pyrazol-5-yl)piperidine-1-carboxylate (17 g, 52.6 mmol) was dissolved in DCM (85 mL) at 0° C. TFA (85 mL, 52.6 mmol, Symax Laboratories) was added dropwise over 15 min and the reaction mixture was stirred at rt for 2 h. The reaction mixture was concentrated under reduced pressure and co-evaporated with toluene (100 mL), and triturated with diethyl ether (100 mL) to give 4-(1-(2-methoxyethyl)-4-methyl-1H-pyrazol-5-yl)piperidine trifluoroacetate salt. The crude residue was taken to next step without further purification. m/z (ESI): 224.3 (M+H)⁺

Step 2. To a solution of 4-(1-(2-methoxyethyl)-4-methyl-1H-pyrazol-5-yl)piperidine trifluoroacetate salt (16 g, 49.9 mmol) in DMA (80 mL) at 0° C. was added DIPEA (32.3 g, 250 mmol, Sonia Industries) and tert-butyl 4-((2R,3R)-1-(6-chloro-2-(trifluoromethyl)pyrimidin-4-yl)-2-methyl)azetidin-3-yl)piperazine-1-carboxylate (21.77 g, 49.9 mmol) and the reaction mixture was stirred at 110° C. for 16 h. The reaction mixture was quenched with ice water (100 mL) and the precipitate was collected by filtration, washed with H₂O (200 mL), and dried under vacuum. The crude residue was purified by chromatography (silica, 0 to 70% EtOAc:hexanes) to give tert-butyl 4-((2R,3R)-1-(6-(4-(1-(2-methoxyethyl)-4-methyl-1H-pyrazol-5-yl)piperidin-1-yl)-2-(trifluoromethyl)pyrimidin-4-yl)-2-methyl)azetidin-3-yl)piperazine-1-carboxylate. m/z (ESI): 623.2 (M+H)⁺. ¹H NMR (400 MHz, DMSO-d₆) δ ppm 7.13 (s, 1H), 5.62 (s, 1H), 4.3-4.5 (m, 3H), 4.23 (t, 2H, J=5.4 Hz), 3.94 (t, H J=8.3 Hz), 3.84 (dd, 1H, J=8.9, 6.7 Hz), 3.61 (t, 3H J=5.4 Hz), 3.3-3.4 (m, 3H), 3.21 (s, 1H), 3.12 (p, 2H J=8.3 Hz), 2.93 (d, 4H, J=19.6 Hz), 2.24 (s, 3H), 1.97 (d, 4H, J=2.9 Hz), 1.6-1.7 (m, 4H), 1.3-1.4 (m, 12H)

Step 3. tert-Butyl 4-((2R,3R)-1-(6-(4-(1-(2-methoxyethyl)-4-methyl-1H-pyrazol-5-yl)piperidin-1-yl)-2-(trifluoromethyl)pyrimidin-4-yl)-2-methyl)azetidin-3-yl)piperazine-1-carboxylate (17 g, 27.3 mmol) was dissolved in DCM (200 mL) at 0° C. TFA (100 mL, 27.3 mmol, Symax Laboratories) was added dropwise over 15 min and the reaction mixture was stirred at rt for 1 h. The reaction mixture was concentrated under reduced pressure and co-evaporated with toluene (100 mL) and triturated with diethyl ether (50 mL) to give 4-(4-(1-(2-methoxyethyl)-4-methyl-1H-pyrazol-5-yl)piperidin-1-yl)-6-((2R,3R)-2-methyl-3-(piperazin-1-yl)azetidin-1-yl)-2-(trifluoromethyl)pyrimidine. m/z (ESI): 523.2 (M+H)⁺. ¹H NMR (400 MHz, DMSO-d₆) δ ppm 8.71 (s, 2H), 7.16 (s, 1H), 5.64 (s, 1H), 4.4-4.5 (m, 3H), 4.2-4.3 (m, 2H), 3.9-4.1 (m, 1H), 3.8-3.9 (m, 1H), 3.63 (t, 2H, J=5.6 Hz), 3.3-3.4 (m, 2H), 3.13 (br s, 6H), 2.9-3.0 (m, 2 Hz, 2.79 (br s, 4H), 1.97 (s, 3H), 1.76 (br s, 4H), 1.40 (d, 3H, J=6.4 Hz).

Step 4. To a solution of 4-(4-(1-(2-Methoxyethyl)-4-methyl-1H-pyrazol-5-yl)piperidin-1-yl)-6-((2R,3R)-2-methyl-3-(piperazin-1-yl)azetidin-1-yl)-2-(trifluoromethyl)pyrimidine (15 g, 28.7 mmol) in NMP (160 mL, Avra) was added potassium carbonate (19.83 g, 144 mmol, Avra Synthesis) and acryloyl chloride (2.55 mL, 31.6 mmol, Symax Laboratories) at 0° C. The reaction mixture was stirred at 0° C. for 10 min, quenched with ice-cold water (200 mL), and extracted with DCM (800 mL). The organic extract was dried over Na₂SO₄, filtered, and concentrated. The crude material % as directly loaded onto a silica gel precolumn (100 g) and purified by column chromatography using a Biotage Isolera (silica, 1 to 3% MeOH:DCM) to give 1-(4-((2R,3R)-1-(6-(4-(1-(2-methoxyethyl)-4-methyl-1H-pyrazol-5-yl)piperidin-1-yl)-2-(trifluoromethyl)pyrimidin-4-yl)-2-methyl)azetidin-3-yl)piperazin-1-yl)prop-2-en-1-one. m/z (ESI): 577.3 (M+H)⁺. ¹H NMR (400 MHz, DMSO-d₆) δ ppm 7.12 (s, 1H), 6.83 (dd, 1H, J=16.4, 10.4 Hz), 6.13 (dd, 1H, J=20.0, 16.4 Hz), 5.6-5.7 (m, 2H), 4.4-4.5 (m, 3H), 4.24 (t, 2H, J=5.4 Hz), 3.8-3.9 (m, 2H), 3.62 (1, 6H, J=5.4 Hz), 3.2-3.3 (m, 4H), 3.0-3.1 (m, 1H), 2.8-2.9 (m, 2H), 2.3-2.9 (m, 4H), 1.97 (s, 3H), 1.7-1.8 (m, 4H), 1.40 (d, 3H J=6.3 Hz). ¹⁹F NMR (377 MHz, DMSO-d₆) 5 ppm −70.22 (s, 1F).

### Example 2-004: 1-(4-((2R,3R)-1-(2-(Difluoromethyl)-6-(4-(1-(2-methoxyethyl)-4-methyl-1H-pyrazol-5-yl)piperidin-1-yl)pyrimidin-4-yl)-2-methyl)azetidin-3-yl)piperazin-1-yl)prop-2-en-1-one

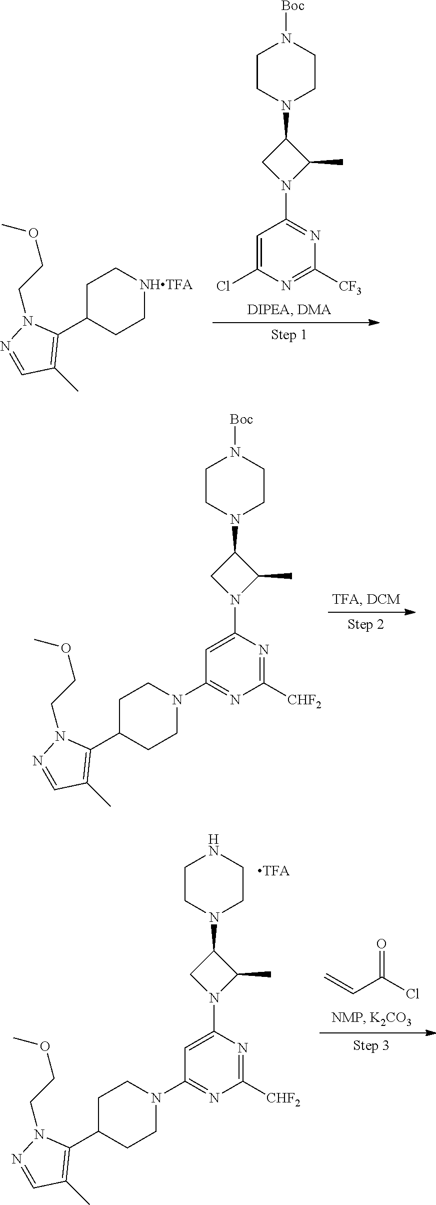

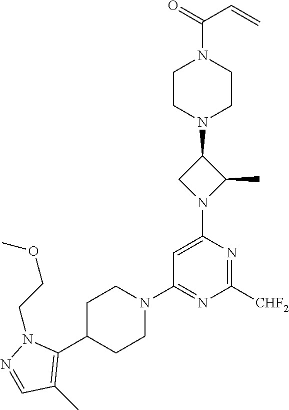 [SMILES: C=CC(=O)N1CCN([C@@H]2CN(c3cc(N4CCC(c5c(C)cnn5CCOC)CC4)nc(C(F)F)n3)[C@@H]2C)CC1]

Step 1. To a mixture of 4-(1-(2-methoxyethyl)-4-methyl-1H-pyrazol-5-yl)piperdine trifluoroacetate salt (11.0 g, 34.3 mmol) and DIPEA (31.3 mL, 172 mmol, Spectrochem) in DMA (110 yl)piperazine-1-carboxylate (14.35 g, 34.3 mmol) at rt and the reaction mixture was stirred at 100° C. for 16 h. the reaction mixture was quenched by the addition of ice-cold water (500 mL), extracted with EtOAc (400 mL), and washed with brine (200 mL). The organic extract was dried over Na₂SO₄, filtered, EtOAc:DCM) to give tert-butyl 4-((2R,3R)-1-(2-(difluoromethyl)-6-(4-(1-(2-methoxyethyl)-4-methyl-1H-pyrazol-5-yl)piperidin-1-yl)pyrimidin-4-yl)-2-methyl)azetidin-3-yl)piperazine-1-carboxylate. m/z (ESI): 605.4 (M+H)⁺. ¹H NMR (400 MHz, DMSO-d₆) δ ppm 7.13 (s, 1H), 6.42 (t, 1H, J=54.9 Hz), 5.55 (s, 1H) 4.47 (q, 3H, J=6.3 Hz), 4.22 (t, 2H, J=5.4 Hz), 3.92 (t, 1H, J=8.2 Hz), 3.82 (dd, 1H, J=8.8, 6.7 Hz), 3.61 (t, 2H, J=5.4 Hz), 3.3 (s, 4H), 3.21 (s, 4H), 3.0-3.1 (m, 1H), 2.7-2.9 (m, 2H), 2.24 (s, 4H), 1.97 (s, 3H), 1.75 (q, 4H, J=8.2, 5.8 Hz), 1.40 (s, 9H), 1.37 (d, 3H, J=0.3 Hz), ¹⁹F NMR (377 MHz, DMSO-d₆): δ−118.71-−118.95 (m, 1F).

Step 2. To a solution of tert-butyl 4-((2R,3R)-1-(2-(difluoromethyl)-6-(4-(1-(2-methoxyethyl)-4-methyl-1H-pyrazol-5-yl)piperidin-1-yl)pyrimidin-4-yl)-2-methyl)azetidin-3-yl)piperazine-1-carboxylate (10 g, 16.54 mmol) in DCM (100 mL) was added TFA (85 mL, 46.4 mmol, Spectrochem) at 0° C., and the reaction mixture was stirred at rt for 3 h. The reaction mixture concentrated under reduced pressure. The crude residue was co-evaporated with toluene (150 mL) and triturated with diethyl ether (150 mL) to give 2-(difluoromethyl)-4-(4-(1-(2-methoxyethyl)-4-methyl-1H-pyrazol-5-yl)piperidin-1-yl)-6-((2R,3R)-2-methyl-3-(piperazin-1-yl)azetidin-1-yl)pyrimidine trifluoroacetate salt. m/z (ESI): 505.2 (M+H)⁺. The crude residue was used in the next step without purification.

Step 3. To a suspension of 2-(difluoromethyl)-4-(4-(1-(2-methoxyethyl)-4-methyl-1H-pyrazol-5-yl)piperidin-1-yl)-6-((2R,3R)-2-methyl-3-(piperazin-1-yl)azetidin-1-yl)pyrimidine trifluoroacetate salt (9.9 g, 16.00 mmol) and potassium carbonate (11 g, 80 mmol, Chempure) in NMP (100 mL) was added a solution of acryloyl chloride (1.422 mL, 17.60 mmol, Symax Laboratories) in NMP (10 mL) at 0° C. This mixture was stirred at 0° C. for 1 h, quenched with ice-cold water (300 mL), extracted with EtOAc (200 mL), and washed with brine (100 mL). The organic extract was dried over Na₂SO₄, filtered, and concentrated under reduced pressure. The crude residue was purified by chromatography (silica, 2 to 4% MeOH:DCM) to give 1-(4-((2R,3R)-1-(2-(difluoromethyl)-6-(4-(1-(2-methoxyethyl)-4-methyl-1H-pyrazol-5-yl)piperidin-1-yl)pyrimidin-4-yl)-2-methyl)azetidin-3-yl)piperazin-1-yl)prop-2-en-1-one. m/z (ESI): 559.2 (M+H)⁺. ¹H NMR (DMSO-d₆, 400 MHz): δ ppm 7.13 (s, 1H), 6.80 (dd, 1H, J=16.7, 10.5 Hz), 6.42 (t, 1H, J=54.9 Hz), 6.11 (dd, 1H, J=16.7, 2.4 Hz), 5.69 (dd, 1H, J=10.4, 2.4 Hz), 5.56 (s, 1H), 4.47 (dt, 3H, J=12.9, 5.6 Hz), 4.23 (t, 2H, J=5.4 Hz), 3.93 (t, 1H, J=8.2 Hz), 3.85 (dd, 1H, J=8.8, 6.7 Hz), 3.61 (t, 6H, J=5.4 Hz), 3.21 (s, 4H), 3.0-3.1 (m, 1H), 2.87 (ddt, 2H, J=15.6, 7.7, 4.3 Hz), 2.29 (s, 4H), 1.97 (s, 3H), 1.75 (h, 4H, J=4.0, 3.3 Hz), 1.39 (d, 3H, J=6.3 Hz). ¹⁹F NMR (377 MHz, DMSO-d₆) δ ppm −118.71-−118.90 (m, 1F).

Examples in the table below were prepared in a manner similar to that described above using the indicated pyrimidinyl halide. The solvent for Step 1 was either DMF or DMA.

| | | | | m/z |
|---|---|---|---|---|
| | | | Pyrimidinyl | (ESI): |
| Ex. | Chemical Structure | Name | halide | (M + H)⁺ |
| 2-005 | 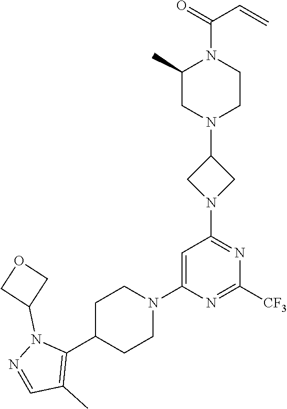 [SMILES: C=CC(=O)N1CCN(C2CN(c3cc(N4CCC(c5c(C)cnn5C5COC5)CC4)nc(C(F)(F)F)n3)C2)C[C@H]1C] | (R)-1-(2-methyl-4-(1-(6-(4-(4- methyl-1-(oxetan-3-yl)-1H- pyrazol-5-yl)piperidin-1-yl)-2- (trifluoromethyl)pyrimidin-4- yl)azetidin-3-yl)piperazin-1- yl)prop-2-en-1-one | E-4 | 575.2 |
| 2-006 | 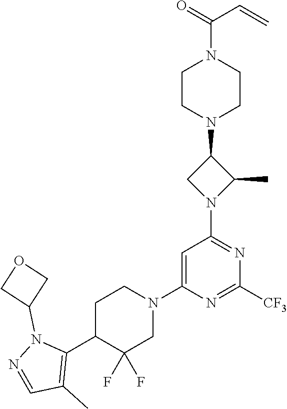 [SMILES: C=CC(=O)N1CCN([C@@H]2CN(c3cc(N4CCC(c5c(C)cnn5C5COC5)C(F)(F)C4)nc(C(F)(F)F)n3)[C@@H]2C)CC1] | 1-(4-((2R,3R)-1-(6-(3,3- difluoro-4-(4-methyl-1- (oxetan-3-yl)-1H-pyrazol-5- yl)piperidin-1-yl)-2- (trifluoromethyl)pyrimidin-4- yl)-2-methylazetidin-3- yl)piperazin-1-yl)prop-2-en-1- one | E-2 | 611.2 |
| 2-007 | 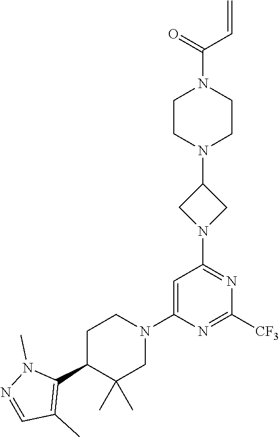 [SMILES: C=CC(=O)N1CCN(C2CN(c3cc(N4CC[C@H](c5c(C)cnn5C)C(C)(C)C4)nc(C(F)(F)F)n3)C2)CC1] | (S)-1-(4-(1-(6-(4-(1,4- dimethyl-1H-pyrazol-5-yl)- 3,3-dimethylpiperidin-1-yl)-2- (trifluoromethyl)pyrimidin-4- yl)azetidin-3-yl)piperazin-1- yl)prop-2-en-1-one | E-3 | 547.1 |
| 2-008 | 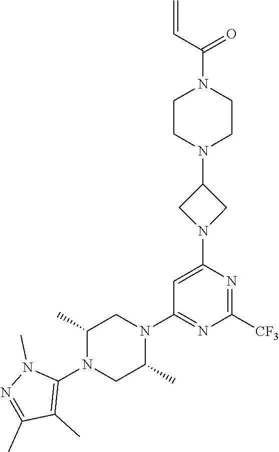 [SMILES: C=CC(=O)N1CCN(C2CN(c3cc(N4C[C@@H](C)N(c5c(C)c(C)nn5C)C[C@H]4C)nc(C(F)(F)F)n3)C2)CC1] | 1-(4-(1-(6-((2R,5R)-2,5- dimethyl-4-(1,3,4-trimethyl- 1H-pyrazol-5-yl)piperazin-1- yl)-2- (trifluoromethyl)pyrimidin-4- yl)azetidin-3-yl)piperazin-1- yl)prop-2-en-1-one | E-3 | 562.3 |
| 2-009 | 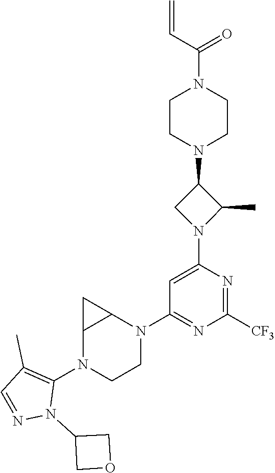 [SMILES: C=CC(=O)N1CCN([C@@H]2CN(c3cc(N4CCN(c5c(C)cnn5C5COC5)C5CC54)nc(C(F)(F)F)n3)[C@@H]2C)CC1] | 1-(4-((2R,3R)-2-methyl-1-(6- (5-(4-methyl-1-(oxetan-3-yl)- 1H-pyrazol-5-yl)-2,5- diazabicyclo[4.1.0]heptan-2- yl)-2- (trifluoromethyl)pyrimidin-4- yl)azetidin-3-yl)piperazin-1- yl)prop-2-en-1-one | E-8 | 588.4 |
| 2-010 | 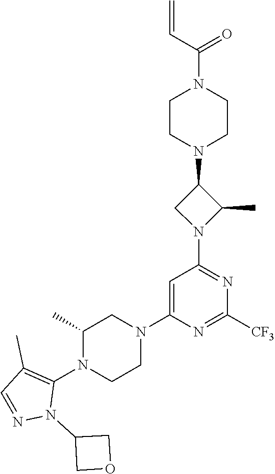 [SMILES: C=CC(=O)N1CCN([C@@H]2CN(c3cc(N4CCN(c5c(C)cnn5C5COC5)[C@H](C)C4)nc(C(F)(F)F)n3)[C@@H]2C)CC1] | 1-(4-((2R,3R)-2-methyl-1-(6- ((R)-3-methyl-4-(4-methyl-1- (oxetan-3-yl)-1H-pyrazol-5- yl)piperazin-1-yl)-2- (trifluoromethyl)pyrimidin-4- yl)azetidin-3-yl)piperazin-1- yl)prop-2-en-1-one | E-8 | 590.4 |
| 2-011 | 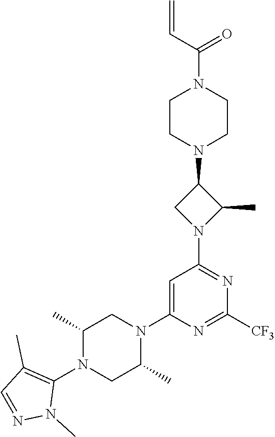 [SMILES: C=CC(=O)N1CCN([C@@H]2CN(c3cc(N4C[C@@H](C)N(c5c(C)cnn5C)C[C@H]4C)nc(C(F)(F)F)n3)[C@@H]2C)CC1] | 1-(4-((2R,3R)-1-(6-((2R,5R)- 4-(1,4-dimethyl-1H-pyrazol-5- yl)-2,5-dimethylpiperazin-1- yl)-2- (trifluoromethyl)pyrimidin-4- yl)-2-methylazetidin-3- yl)piperazin-1-yl)prop-2-en-1- one | E-1 | 562.2 |
| 2-012 | 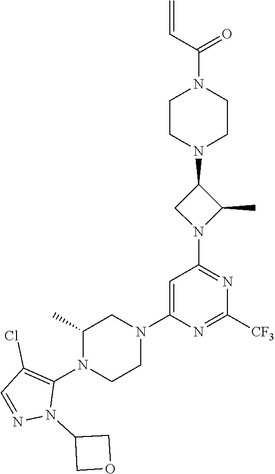 [SMILES: C=CC(=O)N1CCN([C@@H]2CN(c3cc(N4CCN(c5c(Cl)cnn5C5COC5)[C@H](C)C4)nc(C(F)(F)F)n3)[C@@H]2C)CC1] | 1-(4-((2R,3R)-1-(6-((R)-4-(4- chloro-1-(oxetan-3-yl)-1H- pyrazol-5-yl)-3- methylpiperazin-1-yl)-2- (trifluoromethyl)pyrimidin-4- yl)-2-methylazetidin-3- yl)piperazin-1-yl)prop-2-en-1- one | E-8 | 610.2 |
| 2-013 | 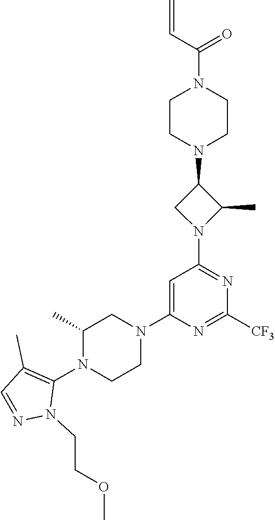 [SMILES: C=CC(=O)N1CCN([C@@H]2CN(c3cc(N4CCN(c5c(C)cnn5CCOC)[C@H](C)C4)nc(C(F)(F)F)n3)[C@@H]2C)CC1] | 1-(4-((2R,3R)-1-(6-((R)-4-(1- (2-methoxyethyl)-4-methyl- 1H-pyrazol-5-yl)-3- methylpiperazin-1-yl)-2- (trifluoromethyl)pyrimidin-4- yl)-2-methylazetidin-3- yl)piperazin-1-yl)prop-2-en-1- one | E-8 | 592.0 |
| 2-014 | 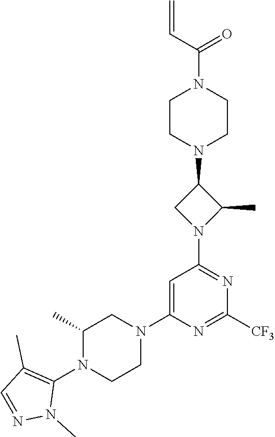 [SMILES: C=CC(=O)N1CCN([C@@H]2CN(c3cc(N4CCN(c5c(C)cnn5C)[C@H](C)C4)nc(C(F)(F)F)n3)[C@@H]2C)CC1] | 1-(4-((2R,3R)-1-(6-((R)-4- (1,4-dimethyl-1H-pyrazol-5- yl)-3-methylpiperazin-1-yl)-2- (trifluoromethyl)pyrimidin-4- yl)-2-methylazetidin-3- yl)piperazin-1-yl)prop-2-en-1- one | E-8 | 548.0 |
| 2-015 | 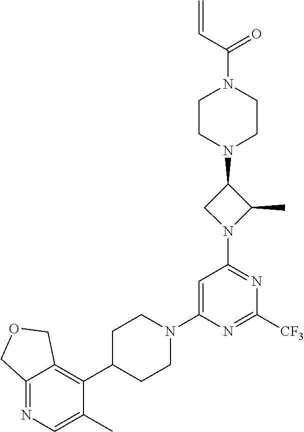 [SMILES: C=CC(=O)N1CCN([C@@H]2CN(c3cc(N4CCC(c5c(C)cnc6c5COC6)CC4)nc(C(F)(F)F)n3)[C@@H]2C)CC1] | 1-(4-((2R,3R)-2-methyl-1-(6- (4-(3-methyl-5,7- dihydrofuro[3,4-b]pyridin-4- yl)piperidin-1-yl)-2- (trifluoromethyl)pyrimidin-4- yl)azetidin-3-yl)piperazin-1- yl)prop-2-en-1-one | E-8 | 572.4 |
| 2-017 | 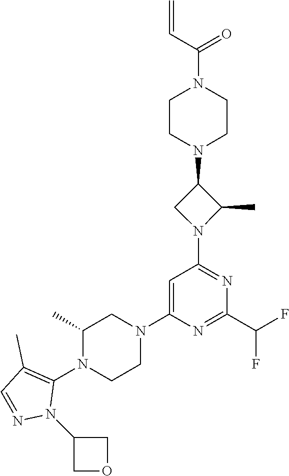 [SMILES: C=CC(=O)N1CCN([C@@H]2CN(c3cc(N4CCN(c5c(C)cnn5C5COC5)[C@H](C)C4)nc(C(F)F)n3)[C@@H]2C)CC1] | 1-(4-((2R,3R)-1-(2- (difluoromethyl)-6-((R)-3- methyl-4-(4-methyl-1- (oxetan-3-yl)-1H-pyrazol-5- yl)piperazin-1-yl)pyrimidin-4- yl)-2-methylazetidin-3- yl)piperazin-1-yl)prop-2-en-1- one | E-2 | 571.9 |
| 2-018 | 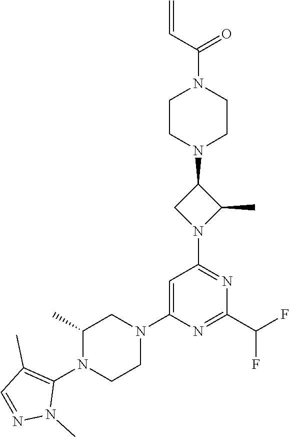 [SMILES: C=CC(=O)N1CCN([C@@H]2CN(c3cc(N4CCN(c5c(C)cnn5C)[C@H](C)C4)nc(C(F)F)n3)[C@@H]2C)CC1] | 1-(4-((2R,3R)-1-(2- (difluoromethyl)-6-((R)-4- (1,4-dimethyl-1H-pyrazol-5- yl)-3-methylpiperazin-1- yl)pyrimidin-4-yl)-2- methylazetidin-3-yl)piperazin- 1-yl)prop-2-en-1-one | -E-2 | 530.0 |
| 2-019 | 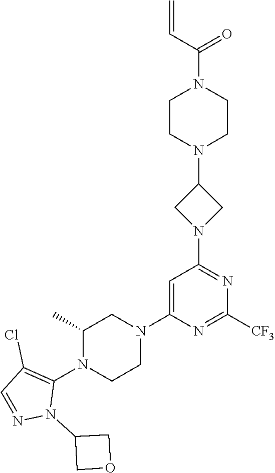 [SMILES: C=CC(=O)N1CCN(C2CN(c3cc(N4CCN(c5c(Cl)cnn5C5COC5)[C@H](C)C4)nc(C(F)(F)F)n3)C2)CC1] | (R)-1-(4-(1-(6-(4-(4-chloro-1- (oxetan-3-yl)-1H-pyrazol-5- yl)-3-methylpiperazin-1-yl)-2- (trifluoromethyl)pyrimidin-4- yl)azetidin-3-yl)piperazin-1- yl)prop-2-en-1-one | E-10 | 596.2 |
| 2-020 | 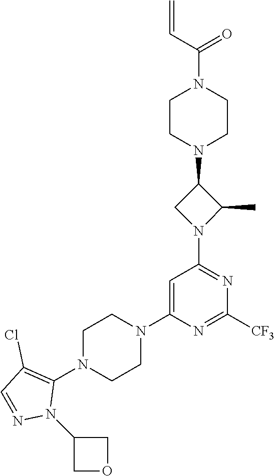 [SMILES: C=CC(=O)N1CCN([C@@H]2CN(c3cc(N4CCN(c5c(Cl)cnn5C5COC5)CC4)nc(C(F)(F)F)n3)[C@@H]2C)CC1] | 1-(4-((2R,3R)-1-(6-(4-(4- chloro-1-(oxetan-3-yl)-1H- pyrazol-5-yl)piperazin-1-yl)- 2-(trifluoromethyl)pyrimidin- 4-yl)-2-methylazetidin-3- yl)piperazin-1-yl)prop-2-en-1- one | E-8 | 596.2 |
| 2-021 | 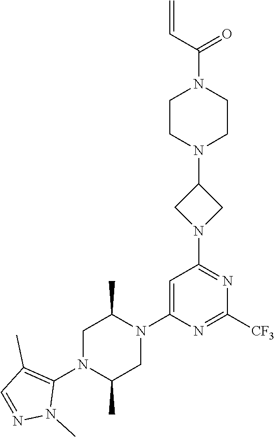 [SMILES: C=CC(=O)N1CCN(C2CN(c3cc(N4C[C@@H](C)N(c5c(C)cnn5C)C[C@H]4C)nc(C(F)(F)F)n3)C2)CC1] | 1-(4-(1-(6-((2R,5R)-4-(1,4- dimethyl-1H-pyrazol-5-yl)- 2,5-dimethylpiperazin-1-yl)-2- (trifluoromethyl)pyrimidin-4- yl)azetidin-3-yl)piperazin-1- yl)prop-2-en-1-one | E-3 | 547.9 |
| 2-022 | 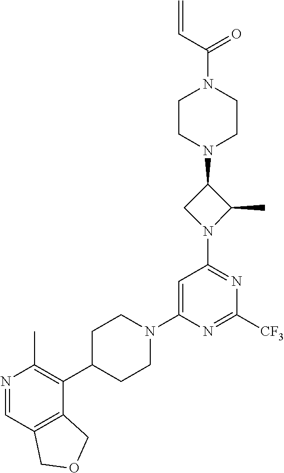 [SMILES: C=CC(=O)N1CCN([C@@H]2CN(c3cc(N4CCC(c5c(C)ncc6c5COC6)CC4)nc(C(F)(F)F)n3)[C@@H]2C)CC1] | 1-(4-((2R,3R)-2-methyl-1-(6- (4-(6-methyl-1,3- dihydrofuro[3,4-c]pyridin-7- yl)piperidin-1-yl)-2- (trifluoromethyl)pyrimidin-4- yl)azetidin-3-yl)piperazin-1- yl)prop-2-en-1-one | E-8 | 572.2 |
| 2-023 | 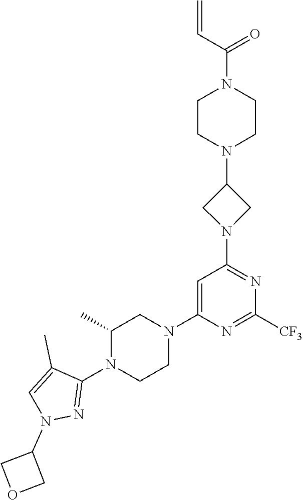 [SMILES: C=CC(=O)N1CCN(C2CN(c3cc(N4CCN(c5nn(C6COC6)cc5C)[C@H](C)C4)nc(C(F)(F)F)n3)C2)CC1] | (R)-1-(4-(1-(6-(3-methyl-4-(4- methyl-1-(oxetan-3-yl)-1H- pyrazol-3-yl)piperazin-1-yl)- 2-(trifluoromethyl)pyrimidin- 4-yl)azetidin-3-yl)piperazin-1- yl)prop-2-en-1-one | E-3 | 575.9 |
| 2-024 | 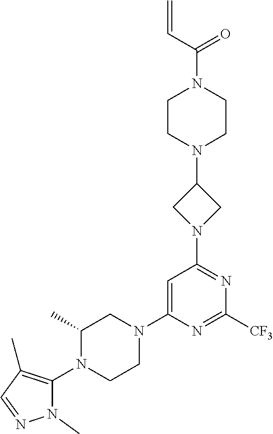 [SMILES: C=CC(=O)N1CCN(C2CN(c3cc(N4CCN(c5c(C)cnn5C)[C@H](C)C4)nc(C(F)(F)F)n3)C2)CC1] | (R)-1-(4-(1-(6-(4-(1,4- dimethyl-1H-pyrazol-5-yl)-3- methylpiperazin-1-yl)-2- (trifluoromethyl)pyrimidin-4- yl)azetidin-3-yl)piperazin-1- yl)prop-2-en-1-one | E-3 | 533.9 |
| 2-025 | 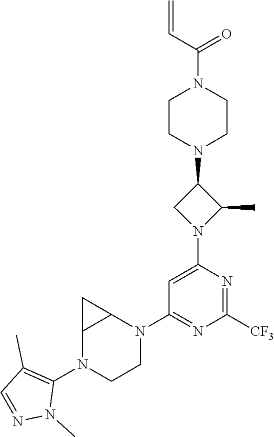 [SMILES: C=CC(=O)N1CCN([C@@H]2CN(c3cc(N4CCN(c5c(C)cnn5C)C5CC54)nc(C(F)(F)F)n3)[C@@H]2C)CC1] | 1-(4-((2R,3R)-1-(6-(5-(1,4- dimethyl-1H-pyrazol-5-yl)- 2,5-diazabicyclo[4.1.0]heptan- 2-yl)-2- (trifluoromethyl)pyrimidin-4- yl)-2-methylazetidin-3- yl)piperazin-1-yl)prop-2-en-1- one | E-1 | 545.9 |
| 2-026 | 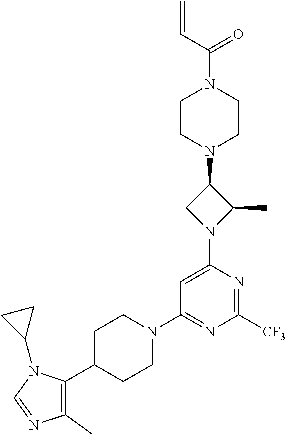 [SMILES: C=CC(=O)N1CCN([C@@H]2CN(c3cc(N4CCC(c5c(C)ncn5C5CC5)CC4)nc(C(F)(F)F)n3)[C@@H]2C)CC1] | 1-(4-((2R,3R)-1-(6-(4-(1- cyclopropyl-4-methyl-1H- imidazol-5-yl)piperidin-1-yl)- 2-(trifluoromethyl)pyrimidin- 4-yl)-2-methylazetidin-3- yl)piperazin-1-yl)prop-2-en-1- one | E-1 | 559.0 |
| 2-027 | 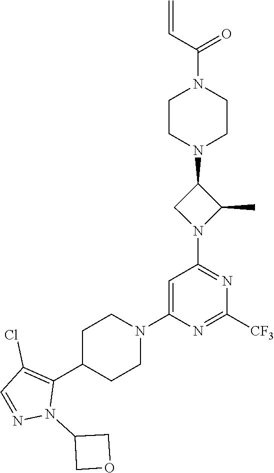 [SMILES: C=CC(=O)N1CCN([C@@H]2CN(c3cc(N4CCC(c5c(Cl)cnn5C5COC5)CC4)nc(C(F)(F)F)n3)[C@@H]2C)CC1] | 1-(4-((2R,3R)-1-(6-(4-(4- chloro-1-(oxetan-3-yl)-1H- pyrazol-5-yl)piperidin-1-yl)-2- (trifluoromethyl)pyrimidin-4- yl)-2-methylazetidin-3- yl)piperazin-1-yl)prop-2-en-1- one | E-1 | 594.9 |
| 2-028 | 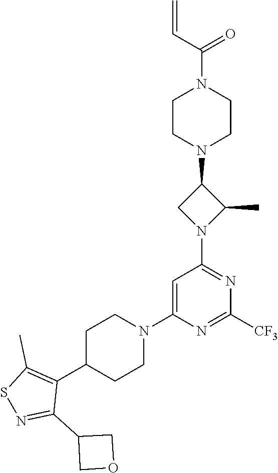 [SMILES: C=CC(=O)N1CCN([C@@H]2CN(c3cc(N4CCC(c5c(C6COC6)nsc5C)CC4)nc(C(F)(F)F)n3)[C@@H]2C)CC1] | 1-(4-((2R,3R)-2-methyl-1-(6- (4-(5-methyl-3-(oxetan-3- yl)isothiazol-4-yl)piperidin-1- yl)-2- (trifluoromethyl)pyrimidin-4- yl)azetidin-3-yl)piperazin-1- yl)prop-2-en-1-one | E-1 | 591.9 |
| 2-029 | 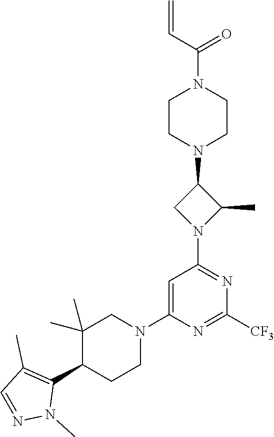 [SMILES: C=CC(=O)N1CCN([C@@H]2CN(c3cc(N4CC[C@@H](c5c(C)cnn5C)C(C)(C)C4)nc(C(F)(F)F)n3)[C@@H]2C)CC1] | 1-(4-((2R,3R)-1-(6-((R)-4- (1,4-dimethyl-1H-pyrazol-5- yl)-3,3-dimethylpiperidin-1- yl)-2- (trifluoromethyl)pyrimidin-4- yl)-2-methylazetidin-3- yl)piperazin-1-yl)prop-2-en-1- one | E-1 | 561.2 |
| 2-030 | 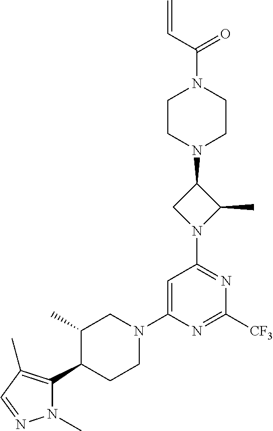 [SMILES: C=CC(=O)N1CCN([C@@H]2CN(c3cc(N4CC[C@@H](c5c(C)cnn5C)[C@H](C)C4)nc(C(F)(F)F)n3)[C@@H]2C)CC1] | 1-(4-((2R,3R)-1-(6- ((3S*,4R*)-4-(1,4-dimethyl- 1H-pyrazol-5-yl)-3- methylpiperidin-1-yl)-2- (trifluoromethyl)pyrimidin-4- yl)-2-methylazetidin-3- yl)piperazin-1-yl)prop-2-en-1- one | E-1 | 547.2 |
| 2-031 | 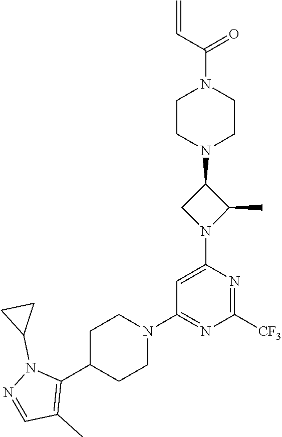 [SMILES: C=CC(=O)N1CCN([C@@H]2CN(c3cc(N4CCC(c5c(C)cnn5C5CC5)CC4)nc(C(F)(F)F)n3)[C@@H]2C)CC1] | 1-(4-((2R,3R)-1-(6-(4-(1- cyclopropyl-4-methyl-1H- pyrazol-5-yl)piperidin-1-yl)-2- (trifluoromethyl)pyrimidin-4- yl)-2-methylazetidin-3- yl)piperazin-1-yl)prop-2-en-1- one | E-1 | 559.0 |
| 2-032 | 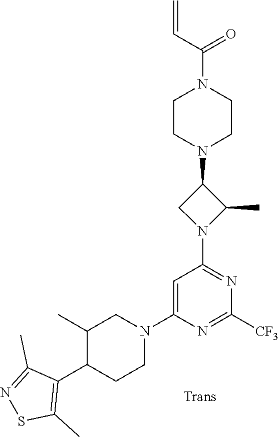 [SMILES: C=CC(=O)N1CCN([C@@H]2CN(c3cc(N4CCC(c5c(C)nsc5C)C(C)C4)nc(C(F)(F)F)n3)[C@@H]2C)CC1] | 1-(4-((2R,3R)-1-(6-(4-(3,5- dimethylisothiazol-4-yl)-3- methylpiperidin-1-yl)-2- (trifluoromethyl)pyrimidin-4- yl)-2-methylazetidin-3- yl)piperazin-1-yl)prop-2-en-1- one | E-1 | 564.2 |
| 2-033 | 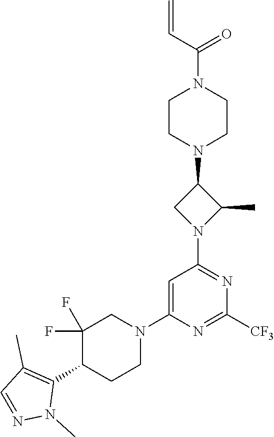 [SMILES: C=CC(=O)N1CCN([C@@H]2CN(c3cc(N4CC[C@H](c5c(C)cnn5C)C(F)(F)C4)nc(C(F)(F)F)n3)[C@@H]2C)CC1] | 1-(4-((2R,3R)-1-(6-((R)-4- (1,4-dimethyl-1H-pyrazol-5- yl)-3,3-difluoropiperidin-1- yl)-2- (trifluoromethyl)pyrimidin-4- yl)-2-methylazetidin-3- yl)piperazin-1-yl)prop-2-en-1- one | E-1 | 569.2 |
| 2-034 | 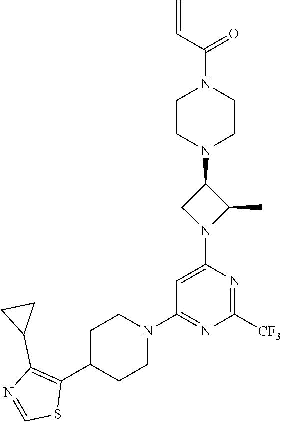 [SMILES: C=CC(=O)N1CCN([C@@H]2CN(c3cc(N4CCC(c5scnc5C5CC5)CC4)nc(C(F)(F)F)n3)[C@@H]2C)CC1] | 1-(4-((2R,3R)-1-(6-(4-(4- cyclopropylthiazol-5- yl)piperidin-1-yl)-2- (trifluoromethyl)pyrimidin-4- yl)-2-methylazetidin-3- yl)piperazin-1-yl)prop-2-en-1- one | E-1 | 562.0 |
| 2-035 | 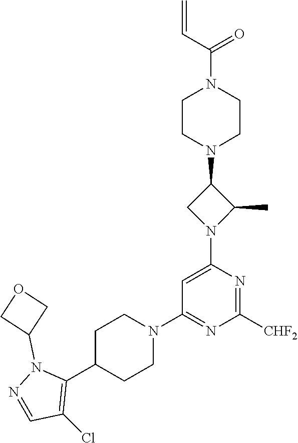 [SMILES: C=CC(=O)N1CCN([C@@H]2CN(c3cc(N4CCC(c5c(Cl)cnn5C5COC5)CC4)nc(C(F)F)n3)[C@@H]2C)CC1] | 1-(4-((2R,3R)-1-(6-(4-(4- chloro-1-(oxetan-3-yl)-1H- pyrazol-5-yl)piperidin-1-yl)-2- (difluoromethyl)pyrimidin-4- yl)-2-methylazetidin-3- yl)piperazin-1-yl)prop-2-en-1- one | E-2 | 577.4 |
| 2-036 | 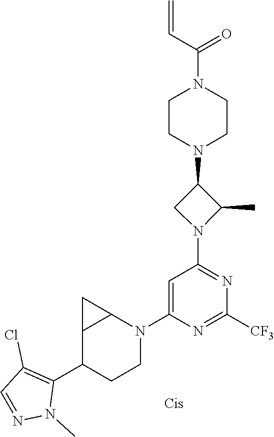 [SMILES: C=CC(=O)N1CCN([C@@H]2CN(c3cc(N4CCC(c5c(Cl)cnn5C)C5CC54)nc(C(F)(F)F)n3)[C@@H]2C)CC1] | 1-(4-((2R,3R)-1-(6-(5-(4- chloro-1-methyl-1H-pyrazol- 5-yl)-2- azabicyclo[4.1.0]heptan-2-yl)- 2-(trifluoromethyl)pyrimidin- 4-yl)-2-methylazetidin-3- yl)piperazin-1-yl)prop-2-en-1- one | E-1 | 565.2 |
| 2-037 | 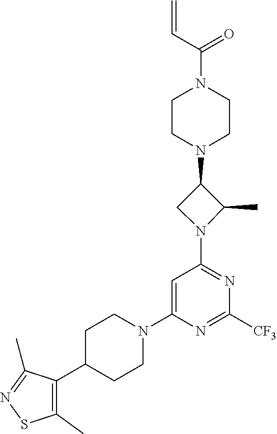 [SMILES: C=CC(=O)N1CCN([C@@H]2CN(c3cc(N4CCC(c5c(C)nsc5C)CC4)nc(C(F)(F)F)n3)[C@@H]2C)CC1] | 1-(4-((2R,3R)-1-(6-(4-(3,5- dimethylisothiazol-4- yl)piperidin-1-yl)-2- (trifluoromethyl)pyrimidin-4- yl)-2-methylazetidin-3- yl)piperazin-1-yl)prop-2-en-1- one | E-1 | 550.2 |
| 2-038 | 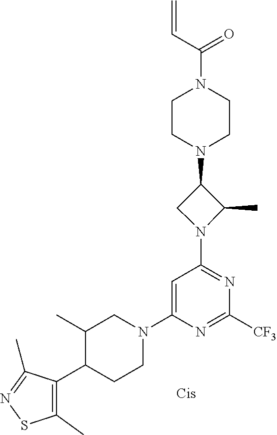 [SMILES: C=CC(=O)N1CCN([C@@H]2CN(c3cc(N4CCC(c5c(C)nsc5C)C(C)C4)nc(C(F)(F)F)n3)[C@@H]2C)CC1] | 1-(4-((2R,3R)-1-(6-(4-(3,5- dimethylisothiazol-4-yl)-3- methylpiperidin-1-yl)-2- (trifluoromethyl)pyrimidin-4- yl)-2-methylazetidin-3- yl)piperazin-1-yl)prop-2-en-1- one | E-1 | 564.2 |
| 2-039 | 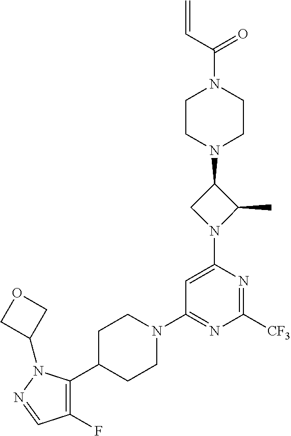 [SMILES: C=CC(=O)N1CCN([C@@H]2CN(c3cc(N4CCC(c5c(F)cnn5C5COC5)CC4)nc(C(F)(F)F)n3)[C@@H]2C)CC1] | 1-(4-((2R,3R)-1-(6-(4-(4- fluoro-1-(oxetan-3-yl)-1H- pyrazol-5-yl)piperidin-1-yl)-2- (trifluoromethyl)pyrimidin-4- yl)-2-methylazetidin-3- yl)piperazin-1-yl)prop-2-en-1- one | E-1 | 579.2 |
| 2-040 | 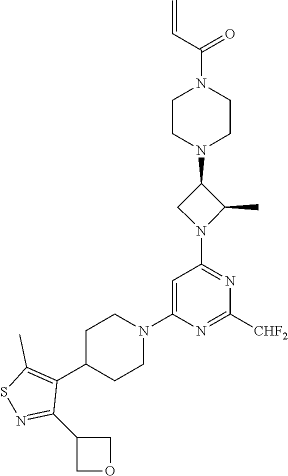 [SMILES: C=CC(=O)N1CCN([C@@H]2CN(c3cc(N4CCC(c5c(C6COC6)nsc5C)CC4)nc(C(F)F)n3)[C@@H]2C)CC1] | 1-(4-((2R,3R)-1-(2- (difluoromethyl)-6-(4-(5- methyl-3-(oxetan-3- yl)isothiazol-4-yl)piperidin-1- yl)pyrimidin-4-yl)-2- methylazetidin-3-yl)piperazin- 1-yl)prop-2-en-1-one | E-2 | 574.2 |
| 2-041 | 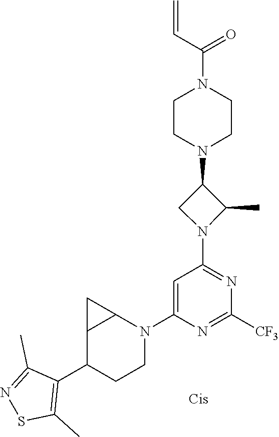 [SMILES: C=CC(=O)N1CCN([C@@H]2CN(c3cc(N4CCC(c5c(C)nsc5C)C5CC54)nc(C(F)(F)F)n3)[C@@H]2C)CC1] | 1-(4-((2R,3R)-1-(6-(5-(3,5- dimethylisothiazol-4-yl)-2- azabicyclo[4.1.0]heptan-2-yl)- 2-(trifluoromethyl)pyrimidin- 4-yl)-2-methylazetidin-3- yl)piperazin-1-yl)prop-2-en-1- one | E-1 | 562.2 |
| 2-042 | 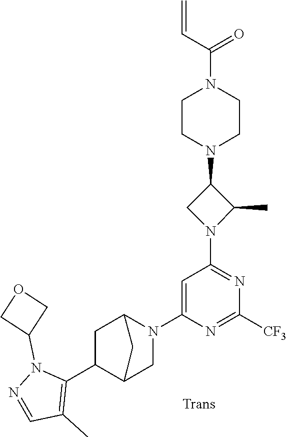 [SMILES: C=CC(=O)N1CCN([C@@H]2CN(c3cc(N4CC5CC4CC5c4c(C)cnn4C4COC4)nc(C(F)(F)F)n3)[C@@H]2C)CC1] | 1-(4-((2R,3R)-2-methyl-1-(6- (5-(4-methyl-1-(oxetan-3-yl)- 1H-pyrazol-5-yl)-2- azabicyclo[2.2.1]heptan-2-yl)- 2-(trifluoromethyl)pyrimidin- 4-yl)azetidin-3-yl)piperazin-1- yl)prop-2-en-1-one | E-1 | 587.2 |
| 2-043 | 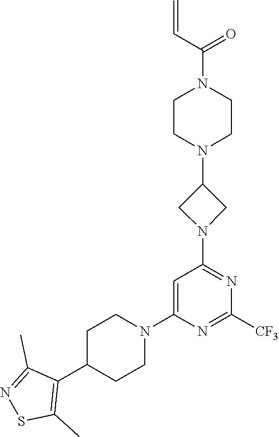 [SMILES: C=CC(=O)N1CCN(C2CN(c3cc(N4CCC(c5c(C)nsc5C)CC4)nc(C(F)(F)F)n3)C2)CC1] | 1-(4-(1-(6-(4-(3,5- dimethylisothiazol-4- yl)piperidin-1-yl)-2- (trifluoromethyl)pyrimidin-4- yl)azetidin-3-yl)piperazin-1- yl)prop-2-en-1-one | E-3 | 536.0 |
| 2-044 | 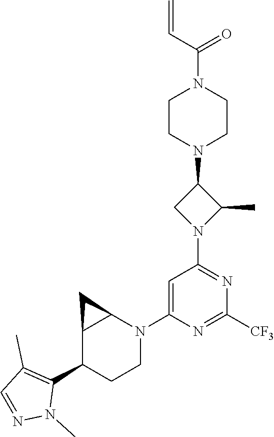 [SMILES: C=CC(=O)N1CCN([C@@H]2CN(c3cc(N4CC[C@@H](c5c(C)cnn5C)[C@H]5C[C@H]54)nc(C(F)(F)F)n3)[C@@H]2C)CC1] | 1-(4-((2R,3R)-1-(6- ((1R,5R,6R)-5-(1,4-dimethyl- 1H-pyrazol-5-yl)-2- azabicyclo[4.1.0]heptan-2-yl)- 2-(trifluoromethyl)pyrimidin- 4-yl)-2-methylazetidin-3- yl)piperazin-1-yl)prop-2-en-1- one | E-1 | 545.2 |
| 2-045 | 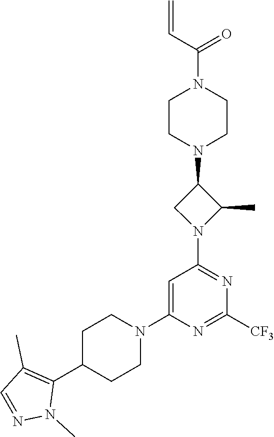 [SMILES: C=CC(=O)N1CCN([C@@H]2CN(c3cc(N4CCC(c5c(C)cnn5C)CC4)nc(C(F)(F)F)n3)[C@@H]2C)CC1] | 1-(4-((2R,3R)-1-(6-(4-(1,4- dimethyl-1H-pyrazol-5- yl)piperidin-1-yl)-2- (trifluoromethyl)pyrimidin-4- yl)-2-methylazetidin-3- yl)piperazin-1-yl)prop-2-en-1- one | E-1 | 533.2 |
| 2-046 | 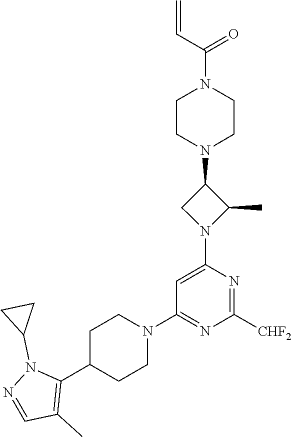 [SMILES: C=CC(=O)N1CCN([C@@H]2CN(c3cc(N4CCC(c5c(C)cnn5C5CC5)CC4)nc(C(F)F)n3)[C@@H]2C)CC1] | 1-(4-((2R,3R)-1-(6-(4-(1- cyclopropyl-4-methyl-1H- pyrazol-5-yl)piperidin-1-yl)-2- (difluoromethyl)pyrimidin-4- yl)-2-methylazetidin-3- yl)piperazin-1-yl)prop-2-en-1- one | E-2 | 541.3 |
| 2-047 | 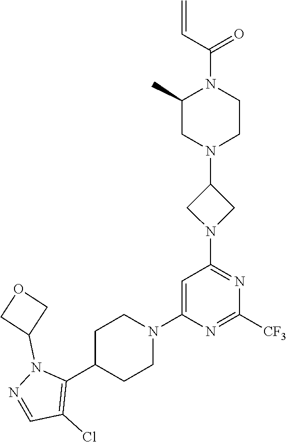 [SMILES: C=CC(=O)N1CCN(C2CN(c3cc(N4CCC(c5c(Cl)cnn5C5COC5)CC4)nc(C(F)(F)F)n3)C2)C[C@H]1C] | (R)-1-(4-(1-(6-(4-(4-chloro-1- (oxetan-3-yl)-1H-pyrazol-5- yl)piperidin-1-yl)-2- (trifluoromethyl)pyrimidin-4- yl)azetidin-3-yl)-2- methylpiperazin-1-yl)prop-2- en-1-one | E-4 | 595.2 |
| 2-048 | 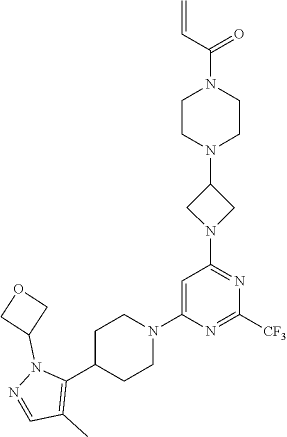 [SMILES: C=CC(=O)N1CCN(C2CN(c3cc(N4CCC(c5c(C)cnn5C5COC5)CC4)nc(C(F)(F)F)n3)C2)CC1] | 1-(4-(1-(6-(4-(4-methyl-1- (oxetan-3-yl)-1H-pyrazol-5- yl)piperidin-1-yl)-2- (trifluoromethyl)pyrimidin-4- yl)azetidin-3-yl)piperazin-1- yl)prop-2-en-1-one | E-3 | 560.7 |
| 2-049 | 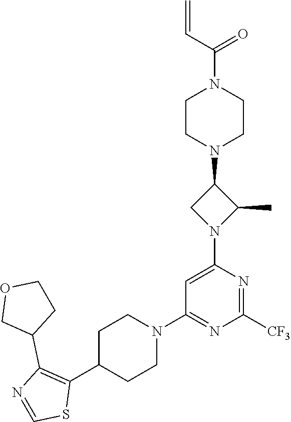 [SMILES: C=CC(=O)N1CCN([C@@H]2CN(c3cc(N4CCC(c5scnc5C5CCOC5)CC4)nc(C(F)(F)F)n3)[C@@H]2C)CC1] | 1-(4-((2R,3R)-2-methyl-1-(6- (4-(4-(tetrahydrofuran-3- yl)thiazol-5-yl)piperidin-1-yl)- 2-(trifluoromethyl)pyrimidin- 4-yl)azetidin-3-yl)piperazin-1- yl)prop-2-en-1-one | E-1 | 592.2 |
| 2-050 | 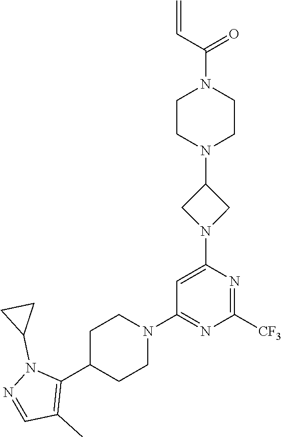 [SMILES: C=CC(=O)N1CCN(C2CN(c3cc(N4CCC(c5c(C)cnn5C5CC5)CC4)nc(C(F)(F)F)n3)C2)CC1] | 1-(4-(1-(6-(4-(1-cyclopropyl- 4-methyl-1H-pyrazol-5- yl)piperidin-1-yl)-2- (trifluoromethyl)pyrimidin-4- yl)azetidin-3-yl)piperazin-1- yl)prop-2-en-1-one | E-3 | 545.0 |
| 2-051 | 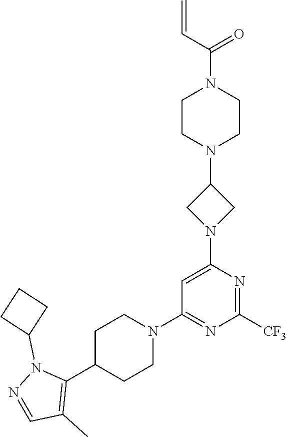 [SMILES: C=CC(=O)N1CCN(C2CN(c3cc(N4CCC(c5c(C)cnn5C5CCC5)CC4)nc(C(F)(F)F)n3)C2)CC1] | 1-(4-(1-(6-(4-(1-cyclobutyl-4- methyl-1H-pyrazol-5- yl)piperidin-1-yl)-2- (trifluoromethyl)pyrimidin-4- yl)azetidin-3-yl)piperazin-1- yl)prop-2-en-1-one | E-3 | 558.8 |
| 2-052 | 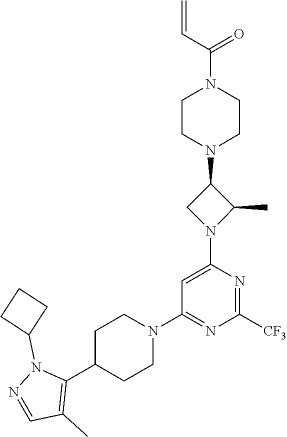 [SMILES: C=CC(=O)N1CCN([C@@H]2CN(c3cc(N4CCC(c5c(C)cnn5C5CCC5)CC4)nc(C(F)(F)F)n3)[C@@H]2C)CC1] | 1-(4-((2R,3R)-1-(6-(4-(1- cyclobutyl-4-methyl-1H- pyrazol-5-yl)piperidin-1-yl)-2- (trifluoromethyl)pyrimidin-4- yl)-2-methylazetidin-3- yl)piperazin-1-yl)prop-2-en-1- one | E-1 | 572.7 |
| 2-053 | 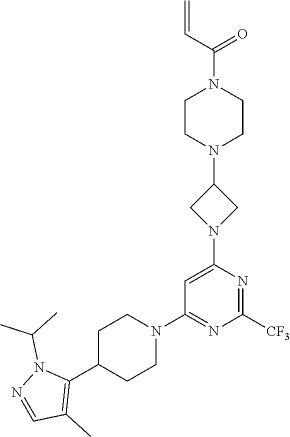 [SMILES: C=CC(=O)N1CCN(C2CN(c3cc(N4CCC(c5c(C)cnn5C(C)C)CC4)nc(C(F)(F)F)n3)C2)CC1] | 1-(4-(1-(6-(4-(1-isopropyl-4- methyl-1H-pyrazol-5- yl)piperidin-1-yl)-2- (trifluoromethyl)pyrimidin-4- yl)azetidin-3-yl)piperazin-1- yl)prop-2-en-1-one | E-3 | 547.1 |
| 2-054 | 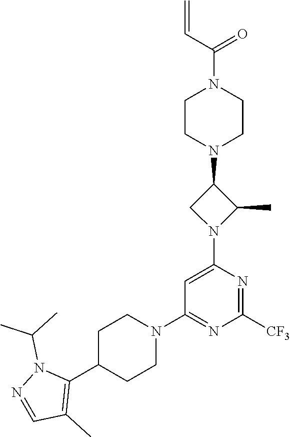 [SMILES: C=CC(=O)N1CCN([C@@H]2CN(c3cc(N4CCC(c5c(C)cnn5C(C)C)CC4)nc(C(F)(F)F)n3)[C@@H]2C)CC1] | 1-(4-((2R,3R)-1-(6-(4-(1- isopropyl-4-methyl-1H- pyrazol-5-yl)piperidin-1-yl)-2- (trifluoromethyl)pyrimidin-4- yl)-2-methylazetidin-3- yl)piperazin-1-yl)prop-2-en-1- one | E-1 | 561.2 |
| 2-055 | 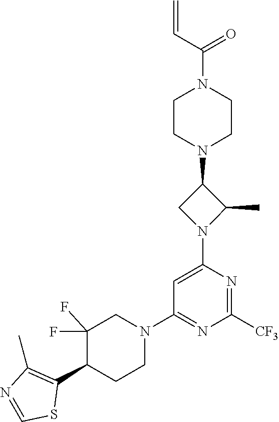 [SMILES: C=CC(=O)N1CCN([C@@H]2CN(c3cc(N4CC[C@@H](c5scnc5C)C(F)(F)C4)nc(C(F)(F)F)n3)[C@@H]2C)CC1] | 1-(4-((2R,3R)-1-(6-((R)-3,3- difluoro-4-(4-methylthiazol-5- yl)piperidin-1-yl)-2- (trifluoromethyl)pyrimidin-4- yl)-2-methylazetidin-3- yl)piperazin-1-yl)prop-2-en-1- one | E-1 | 572.2 |
| 2-056 | 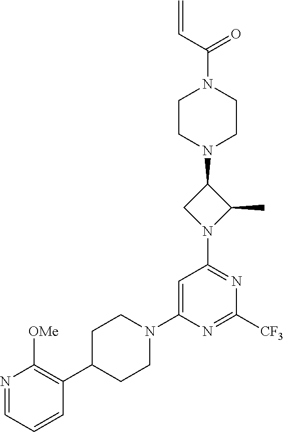 [SMILES: C=CC(=O)N1CCN([C@@H]2CN(c3cc(N4CCC(c5cccnc5OC)CC4)nc(C(F)(F)F)n3)[C@@H]2C)CC1] | 1-(4-((2R,3R)-1-(6-(4-(2- methoxypyridin-3- yl)piperidin-1-yl)-2- (trifluoromethyl)pyrimidin-4- yl)-2-methylazetidin-3- yl)piperazin-1-yl)prop-2-en-1- one | E-1 | 546.0 |
| 2-057 | 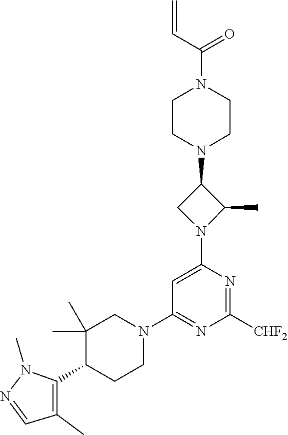 [SMILES: C=CC(=O)N1CCN([C@@H]2CN(c3cc(N4CC[C@H](c5c(C)cnn5C)C(C)(C)C4)nc(C(F)F)n3)[C@@H]2C)CC1] | 1-(4-((2R,3R)-1-(2- (difluoromethyl)-6-((S)-4- (1,4-dimethyl-1H-pyrazol-5- yl)-3,3-dimethylpiperidin-1- yl)pyrimidin-4-yl)-2- methylazetidin-3-yl)piperazin- 1-yl)prop-2-en-1-one | E-2 | 543.3 |
| 2-058 | 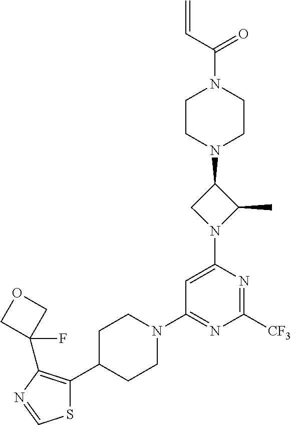 [SMILES: C=CC(=O)N1CCN([C@@H]2CN(c3cc(N4CCC(c5scnc5C5(F)COC5)CC4)nc(C(F)(F)F)n3)[C@@H]2C)CC1] | 1-(4-((2R,3R)-1-(6-(4-(4-(3- fluorooxetan-3-yl)thiazol-5- yl)piperidin-1-yl)-2- (trifluoromethyl)pyrimidin-4- yl)-2-methylazetidin-3- yl)piperazin-1-yl)prop-2-en-1- one | E-1 | 596.3 |
| 2-059 | 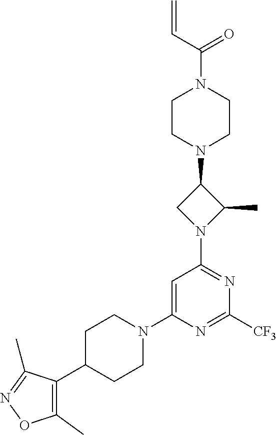 [SMILES: C=CC(=O)N1CCN([C@@H]2CN(c3cc(N4CCC(c5c(C)noc5C)CC4)nc(C(F)(F)F)n3)[C@@H]2C)CC1] | 1-(4-((2R,3R)-1-(6-(4-(3,5- dimethylisoxazol-4- yl)piperidin-1-yl)-2- (trifluoromethyl)pyrimidin-4- yl)-2-methylazetidin-3- yl)piperazin-1-yl)prop-2-en-1- one | E-1 | 534.0 |
| 2-060 | 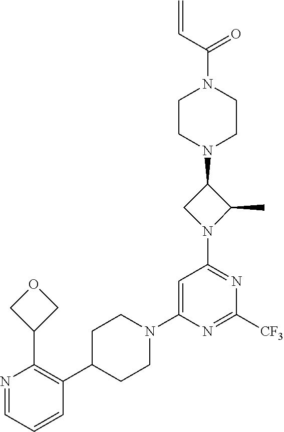 [SMILES: C=CC(=O)N1CCN([C@@H]2CN(c3cc(N4CCC(c5cccnc5C5COC5)CC4)nc(C(F)(F)F)n3)[C@@H]2C)CC1] | 1-(4-((2R,3R)-2-methyl-1-(6- (4-(2-(oxetan-3-yl)pyridin-3- yl)piperidin-1-yl)-2- (trifluoromethyl)pyrimidin-4- yl)azetidin-3-yl)piperazin-1- yl)prop-2-en-1-one | E-1 | 572.1 |
| 2-061 | 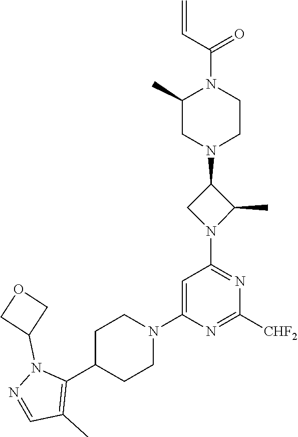 [SMILES: C=CC(=O)N1CCN([C@@H]2CN(c3cc(N4CCC(c5c(C)cnn5C5COC5)CC4)nc(C(F)F)n3)[C@@H]2C)C[C@H]1C] | 1-((R)-4-((2R,3R)-1-(2- (difluoromethyl)-6-(4-(4- methyl-1-(oxetan-3-yl)-1H- pyrazol-5-yl)piperidin-1- yl)pyrimidin-4-yl)-2- methylazetidin-3-yl)-2- methylpiperazin-1-yl)prop-2- en-1-one | E-11 | 570.8 |
| 2-062 | 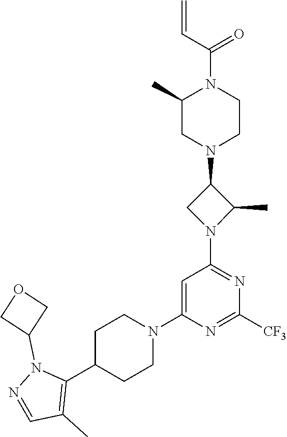 [SMILES: C=CC(=O)N1CCN([C@@H]2CN(c3cc(N4CCC(c5c(C)cnn5C5COC5)CC4)nc(C(F)(F)F)n3)[C@@H]2C)C[C@H]1C] | 1-((R)-2-methyl-4-((2R,3R)-2- methyl-1-(6-(4-(4-methyl-1- (oxetan-3-yl)-1H-pyrazol-5- yl)piperidin-1-yl)-2- (trifluoromethyl)pyrimidin-4- yl)azetidin-3-yl)piperazin-1- yl)prop-2-en-1-one | E-6 | 589.7 |
| 2-063 | 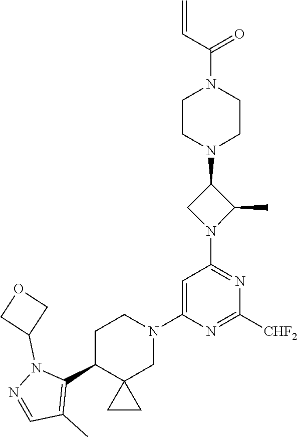 [SMILES: C=CC(=O)N1CCN([C@@H]2CN(c3cc(N4CC[C@H](c5c(C)cnn5C5COC5)C5(CC5)C4)nc(C(F)F)n3)[C@@H]2C)CC1] | 1-(4-((2R,3R)-1-(2- (difluoromethyl)-6-((S)-8-(4- methyl-1-(oxetan-3-yl)-1H- pyrazol-5-yl)-5- azaspiro[2.5]octan-5- yl)pyrimidin-4-yl)-2- methylazetidin-3-yl)piperazin- 1-yl)prop-2-en-1-one | E-2 | 583.2 |
| 2-065 | 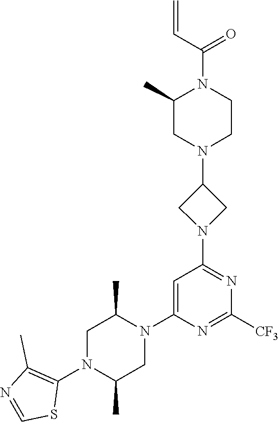 [SMILES: C=CC(=O)N1CCN(C2CN(c3cc(N4C[C@@H](C)N(c5scnc5C)C[C@H]4C)nc(C(F)(F)F)n3)C2)C[C@H]1C] | 1-((R)-4-(1-(6-((2R,5R)-2,5- dimethyl-4-(4-methylthiazol- 5-yl)piperazin-1-yl)-2- (trifluoromethyl)pyrimidin-4- yl)azetidin-3-yl)-2- methylpiperazin-1-yl)prop-2- en-1-one | E-4 | 565.2 |
| 2-066 | 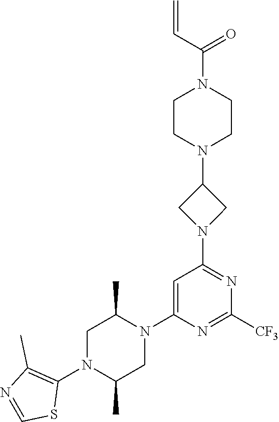 [SMILES: C=CC(=O)N1CCN(C2CN(c3cc(N4C[C@@H](C)N(c5scnc5C)C[C@H]4C)nc(C(F)(F)F)n3)C2)CC1] | 1-(4-(1-(6-((2R,5R)-2,5- dimethyl-4-(4-methylthiazol- 5-yl)piperazin-1-yl)-2- (trifluoromethyl)pyrimidin-4- yl)azetidin-3-yl)piperazin-1- yl)prop-2-en-1-one | E-3 | 551.1 |
| 2-067 | 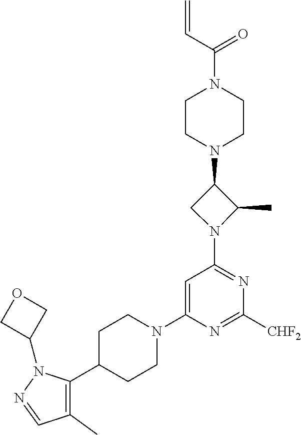 [SMILES: C=CC(=O)N1CCN([C@@H]2CN(c3cc(N4CCC(c5c(C)cnn5C5COC5)CC4)nc(C(F)F)n3)[C@@H]2C)CC1] | 1-(4-((2R,3R)-1-(2- (difluoromethyl)-6-(4-(4- methyl-1-(oxetan-3-yl)-1H- pyrazol-5-yl)piperidin-1- yl)pyrimidin-4-yl)-2- methylazetidin-3-yl)piperazin- 1-yl)prop-2-en-1-one | E-2 | 557.3 |
| 2-068 | 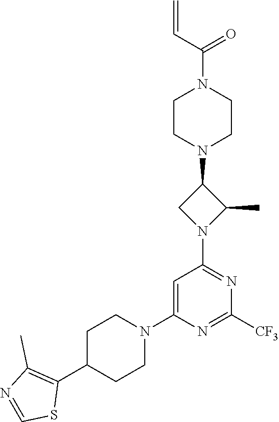 [SMILES: C=CC(=O)N1CCN([C@@H]2CN(c3cc(N4CCC(c5scnc5C)CC4)nc(C(F)(F)F)n3)[C@@H]2C)CC1] | 1-(4-((2R,3R)-2-methyl-1-(6- (4-(4-methylthiazol-5- yl)piperidin-1-yl)-2- (trifluoromethyl)pyrimidin-4- yl)azetidin-3-yl)piperazin-1- yl)prop-2-en-1-one | E-1 | 535.7 |
| 2-069 | 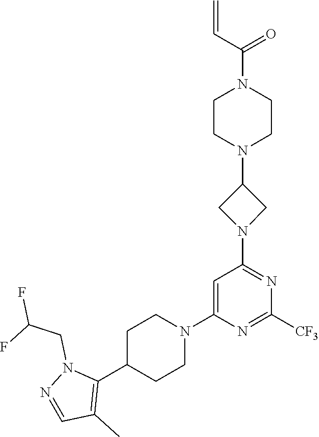 [SMILES: C=CC(=O)N1CCN(C2CN(c3cc(N4CCC(c5c(C)cnn5CC(F)F)CC4)nc(C(F)(F)F)n3)C2)CC1] | 1-(4-(1-(6-(4-(1-(2,2- difluoroethyl)-4-methyl-1H- pyrazol-5-yl)piperidin-1-yl)-2- (trifluoromethyl)pyrimidin-4- yl)azetidin-3-yl)piperazin-1- yl)prop-2-en-1-one | E-10 | 569.1 |
| 2-070 | 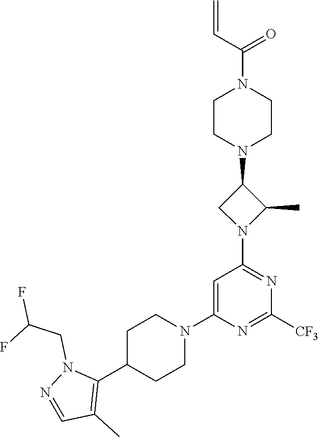 [SMILES: C=CC(=O)N1CCN([C@@H]2CN(c3cc(N4CCC(c5c(C)cnn5CC(F)F)CC4)nc(C(F)(F)F)n3)[C@@H]2C)CC1] | 1-(4-((2R,3R)-1-(6-(4-(1-(2,2- difluoroethyl)-4-methyl-1H- pyrazol-5-yl)piperidin-1-yl)-2- (trifluoromethyl)pyrimidin-4- yl)-2-methylazetidin-3- yl)piperazin-1-yl)prop-2-en-1- one | E-8 | 583.2 |
| 2-071 | 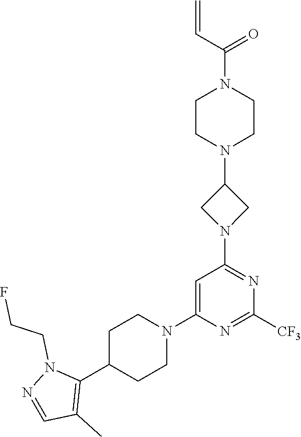 [SMILES: C=CC(=O)N1CCN(C2CN(c3cc(N4CCC(c5c(C)cnn5CCF)CC4)nc(C(F)(F)F)n3)C2)CC1] | 1-(4-(1-(6-(4-(1-(2- fluoroethyl)-4-methyl-1H- pyrazol-5-yl)piperidin-1-yl)-2- (trifluoromethyl)pyrimidin-4- yl)azetidin-3-yl)piperazin-1- yl)prop-2-en-1-one | E-10 | 551.1 |
| 2-072 | 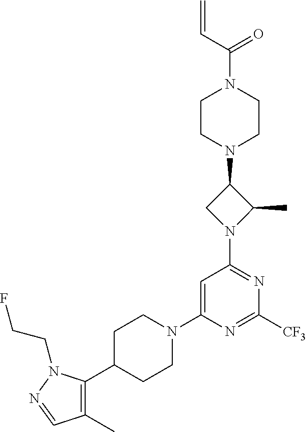 [SMILES: C=CC(=O)N1CCN([C@@H]2CN(c3cc(N4CCC(c5c(C)cnn5CCF)CC4)nc(C(F)(F)F)n3)[C@@H]2C)CC1] | 1-(4-((2R,3R)-1-(6-(4-(1-(2- fluoroethyl)-4-methyl-1H- pyrazol-5-yl)piperidin-1-yl)-2- (trifluoromethyl)pyrimidin-4- yl)-2-methylazetidin-3- yl)piperazin-1-yl)prop-2-en-1- one | E-8 | 565.1 |
| 2-073 | 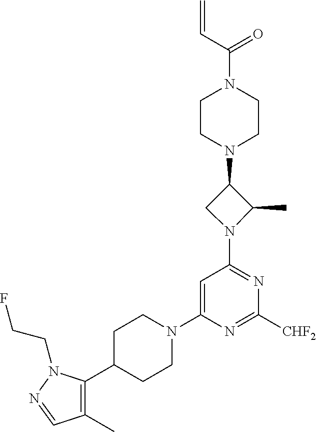 [SMILES: C=CC(=O)N1CCN([C@@H]2CN(c3cc(N4CCC(c5c(C)cnn5CCF)CC4)nc(C(F)F)n3)[C@@H]2C)CC1] | 1-(4-((2R,3R)-1-(2- (difluoromethyl)-6-(4-(1-(2- fluoroethyl)-4-methyl-1H- pyrazol-5-yl)piperidin-1- yl)pyrimidin-4-yl)-2- methylazetidin-3-yl)piperazin- 1-yl)prop-2-en-1-one | E-2 | 547.3 |
| 2-074 | 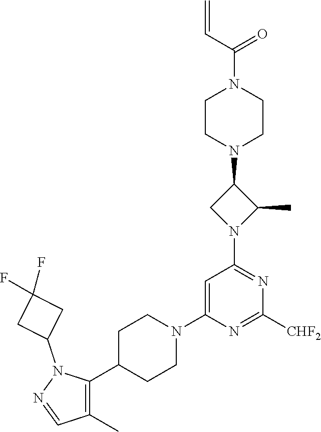 [SMILES: C=CC(=O)N1CCN([C@@H]2CN(c3cc(N4CCC(c5c(C)cnn5C5CC(F)(F)C5)CC4)nc(C(F)F)n3)[C@@H]2C)CC1] | 1-(4-((2R,3R)-1-(6-(4-(1-(3,3- difluorocyclobutyl)-4-methyl- 1H-pyrazol-5-yl)piperidin-1- yl)-2- (difluoromethyl)pyrimidin-4- yl)-2-methylazetidin-3- yl)piperazin-1-yl)prop-2-en-1- one | E-2 | 591.3 |
| 2-075 | 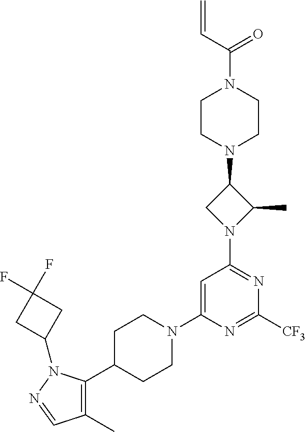 [SMILES: C=CC(=O)N1CCN([C@@H]2CN(c3cc(N4CCC(c5c(C)cnn5C5CC(F)(F)C5)CC4)nc(C(F)(F)F)n3)[C@@H]2C)CC1] | 1-(4-((2R,3R)-1-(6-(4-(1-(3,3- difluorocyclobutyl)-4-methyl- 1H-pyrazol-5-yl)piperidin-1- yl)-2- (trifluoromethyl)pyrimidin-4- yl)-2-methylazetidin-3- yl)piperazin-1-yl)prop-2-en-1- one | E-1 | 609.3 |
| 2-076 | 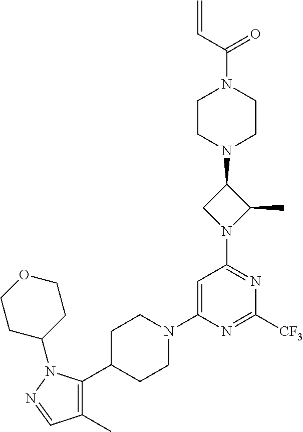 [SMILES: C=CC(=O)N1CCN([C@@H]2CN(c3cc(N4CCC(c5c(C)cnn5C5CCOCC5)CC4)nc(C(F)(F)F)n3)[C@@H]2C)CC1] | 1-(4-((2R,3R)-2-methyl-1-(6- (4-(4-methyl-1-(tetrahydro- 2H-pyran-4-yl)-1H-pyrazol-5- yl)piperidin-1-yl)-2- (trifluoromethyl)pyrimidin-4- yl)azetidin-3-yl)piperazin-1- yl)prop-2-en-1-one | E-8 | 602.7 |
| 2-077 | 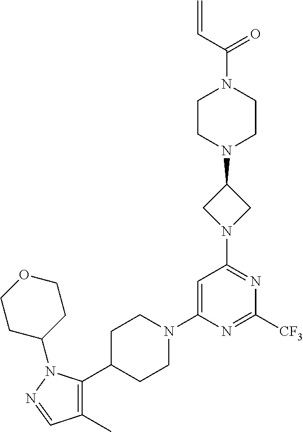 | 1-(4-(1-(6-(4-(4-methyl-1- (tetrahydro-2H-pyran-4-yl)- 1H-pyrazol-5-yl)piperidin-1- yl)-2- (trifluoromethyl)pyrimidin-4- yl)azetidin-3-yl)piperazin-1- yl)prop-2-en-1-one | E-10 | 588.7 |
| 2-078 | 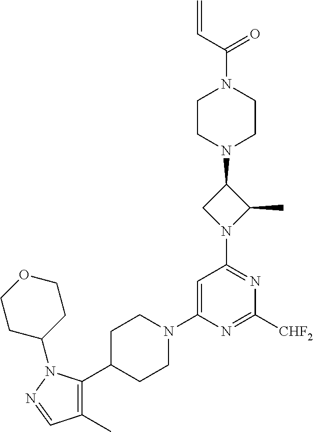 [SMILES: C=CC(=O)N1CCN([C@@H]2CN(c3cc(N4CCC(c5c(C)cnn5C5CCOCC5)CC4)nc(C(F)F)n3)[C@@H]2C)CC1] | 1-(4-((2R,3R)-1-(2- (difluoromethyl)-6-(4-(4- methyl-1-(tetrahydro-2H- pyran-4-yl)-1H-pyrazol-5- yl)piperidin-1-yl)pyrimidin-4- yl)-2-methylazetidin-3- yl)piperazin-1-yl)prop-2-en-1- one | E-9 | 584.7 |
| 2-079 | 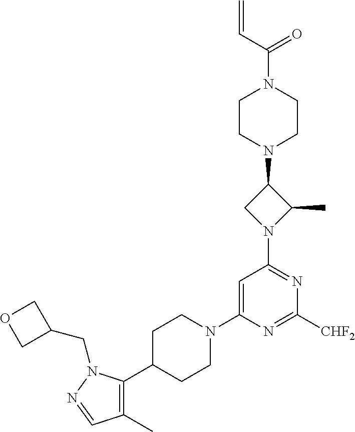 [SMILES: C=CC(=O)N1CCN([C@@H]2CN(c3cc(N4CCC(c5c(C)cnn5CC5COC5)CC4)nc(C(F)F)n3)[C@@H]2C)CC1] | 1-(4-((2R,3R)-1-(2- (difluoromethy)-6-(4-(4- methyl-1-(oxetan-3-ylmethyl)- 1H-pyrazol-5-yl)piperidin-1- yl)pyrimidin-4-yl)-2- methylazetidin-3-yl)piperazin- 1-yl)prop-2-en-1-one | E-2 | 571.2 |
| 2-080 | 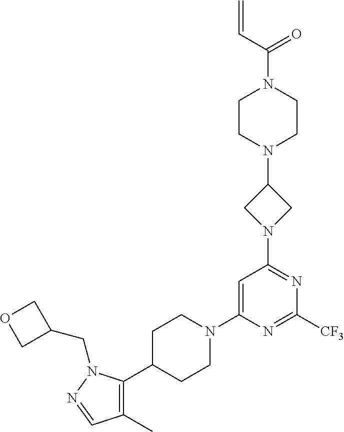 [SMILES: C=CC(=O)N1CCN(C2CN(c3cc(N4CCC(c5c(C)cnn5CC5COC5)CC4)nc(C(F)(F)F)n3)C2)CC1] | 1-(4-(1-(6-(4-(4-methyl-1- (oxetan-3-ylmethyl)-1H- pyrazol-5-yl)piperidin-1-yl)-2- (trifluoromethyl)pyrimidin-4- yl)azetidin-3-yl)piperazin-1- yl)prop-2-en-1-one | E-10 | 575.1 |
| 2-081 | 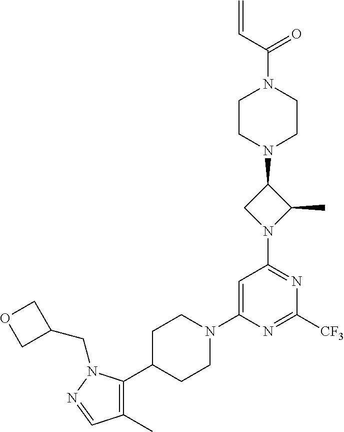 [SMILES: C=CC(=O)N1CCN([C@@H]2CN(c3cc(N4CCC(c5c(C)cnn5CC5COC5)CC4)nc(C(F)(F)F)n3)[C@@H]2C)CC1] | 1-(4-((2R,3R)-2-methyl-1-(6- (4-(4-methyl-1-(oxetan-3- ylmethyl)-1H-pyrazol-5- yl)piperidin-1-yl)-2- (trifluoromethyl)pyrimidin-4- yl)azetidin-3-yl)piperazin-1- yl)prop-2-en-1-one | E-8 | 589.2 |
| 2-082 | 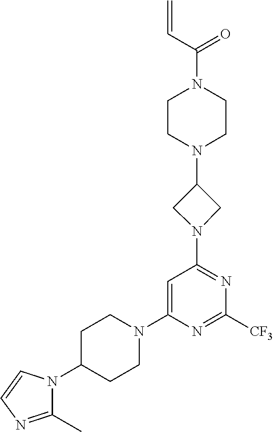 [SMILES: C=CC(=O)N1CCN(C2CN(c3cc(N4CCC(n5ccnc5C)CC4)nc(C(F)(F)F)n3)C2)CC1] | 1-(4-(1-(6-(4-(2-methyl-1H- imidazol-1-yl)piperidin-1-yl)- 2-(trifluoromethyl)pyrimidin- 4-yl)azetidin-3-yl)piperazin-1- yl)prop-2-en-1-one | E-3 | 505.2 |
| 2-083 | 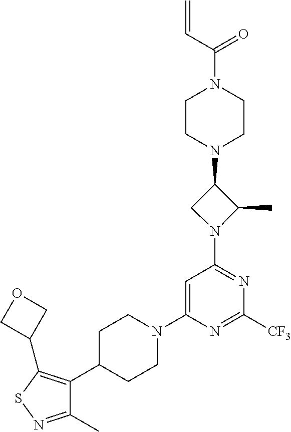 [SMILES: C=CC(=O)N1CCN([C@@H]2CN(c3cc(N4CCC(c5c(C)nsc5C5COC5)CC4)nc(C(F)(F)F)n3)[C@@H]2C)CC1] | 1-(4-((2R,3R)-2-methyl-1-(6- (4-(3-methyl-5-(oxetan-3- yl)isothiazol-4-yl)piperidin-1- yl)-2- (trifluoromethyl)pyrimidin-4- yl)azetidin-3-yl)piperazin-1- yl)prop-2-en-1-one | E-1 | 592.0 |
| 2-084 | 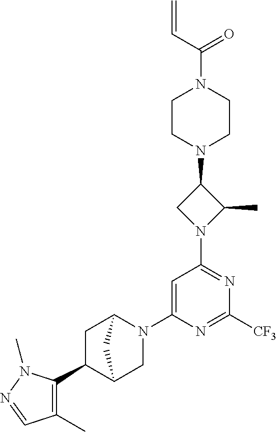 [SMILES: C=CC(=O)N1CCN([C@@H]2CN(c3cc(N4C[C@@H]5C[C@H]4C[C@@H]5c4c(C)cnn4C)nc(C(F)(F)F)n3)[C@@H]2C)CC1] | 1-(4-((2R,3R)-1-(6- ((1S,4R,5S)-5-(1,4-dimethyl- 1H-pyrazol-5-yl)-2- azabicyclo[2.2.1]heptan-2-yl)- 2-(trifluoromethyl)pyrimidin- 4-yl)-2-methylazetidin-3- yl)piperazin-1-yl)prop-2-en-1- one | E-1 | 545.2 |
| 2-085 | 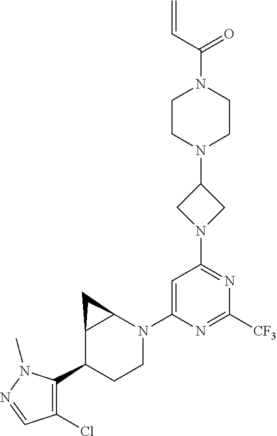 [SMILES: C=CC(=O)N1CCN(C2CN(c3cc(N4CC[C@@H](c5c(Cl)cnn5C)[C@H]5C[C@H]54)nc(C(F)(F)F)n3)C2)CC1] | 1-(4-(1-(6-((1R,5R,6R)-5-(4- chloro-1-methyl-1H-pyrazol- 5-yl)-2- azabicyclo[4.1.0]heptan-2-yl)- 2-(trifluoromethyl)pyrimidin- 4-yl)azetidin-3-yl)piperazin-1- yl)prop-2-en-1-one | E-1 | 551.2 |
| 2-086 | 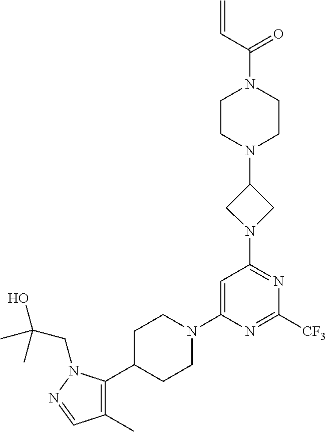 [SMILES: C=CC(=O)N1CCN(C2CN(c3cc(N4CCC(c5c(C)cnn5CC(C)(C)O)CC4)nc(C(F)(F)F)n3)C2)CC1] | 1-(4-(1-(6-(4-(1-(2-hydroxy-2- methylpropyl)-4-methyl-1H- pyrazol-5-yl)piperidin-1-yl)-2- (trifluoromethyl)pyrimidin-4- yl)azetidin-3-yl)piperazin-1- yl)prop-2-en-1-one | E-10 | 576.8 |
| 2-087 | 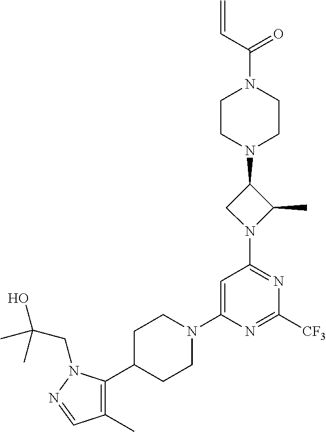 [SMILES: C=CC(=O)N1CCN([C@@H]2CN(c3cc(N4CCC(c5c(C)cnn5CC(C)(C)O)CC4)nc(C(F)(F)F)n3)[C@@H]2C)CC1] | 1-(4-((2R,3R)-1-(6-(4-(1-(2- hydroxy-2-methylpropyl)-4- methyl-1H-pyrazol-5- yl)piperidin-1-yl)-2- (trifluoromethyl)pyrimidin-4- yl)-2-methylazetidin-3- yl)piperazin-1-yl)prop-2-en-1- one | E-8 | 590.7 |
| 2-088 | 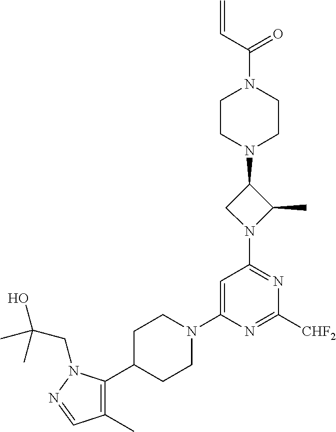 [SMILES: C=CC(=O)N1CCN([C@@H]2CN(c3cc(N4CCC(c5c(C)cnn5CC(C)(C)O)CC4)nc(C(F)F)n3)[C@@H]2C)CC1] | 1-(4-((2R,3R)-1-(2- (difluoromethyl)-6-(4-(1-(2- hydroxy-2-methylpropyl)-4- methyl-1H-pyrazol-5- yl)piperidin-1-yl)pyrimidin-4- yl)-2-methylazetidin-3- yl)piperazin-1-yl)prop-2-en-1- one | E-9 | 573.3 |
| 2-089 | 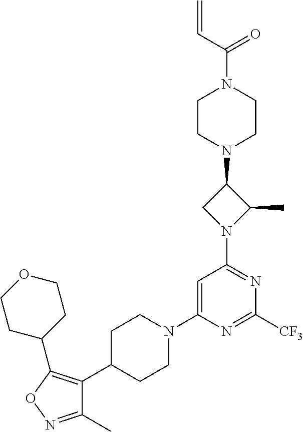 [SMILES: C=CC(=O)N1CCN([C@@H]2CN(c3cc(N4CCC(c5c(C)noc5C5CCOCC5)CC4)nc(C(F)(F)F)n3)[C@@H]2C)CC1] | 1-(4-((2R,3R)-2-methyl-1-(6- (4-(3-methyl-5-(tetrahydro- 2H-pyran-4-yl)isoxazol-4- yl)piperidin-1-yl)-2- (trifluoromethyl)pyrimidin-4- yl)azetidin-3-yl)piperazin-1- yl)prop-2-en-1-one | E-1 | 604.3 |
| 2-090 | 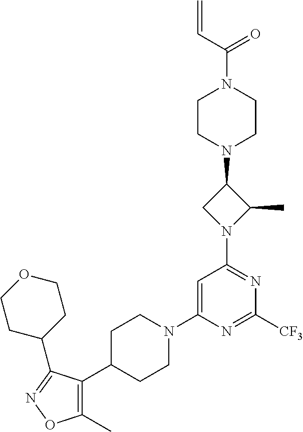 [SMILES: C=CC(=O)N1CCN([C@@H]2CN(c3cc(N4CCC(c5c(C6CCOCC6)noc5C)CC4)nc(C(F)(F)F)n3)[C@@H]2C)CC1] | 1-(4-((2R,3R)-2-methyl-1-(6- (4-(5-methyl-3-(tetrahydro- 2H-pyran-4-yl)isoxazol-4- yl)piperidin-1-yl)-2- (trifluoromethyl)pyrimidin-4- yl)azetidin-3-yl)piperazin-1- yl)prop-2-en-1-one | E-1 | 604.3 |
| 2-091 | 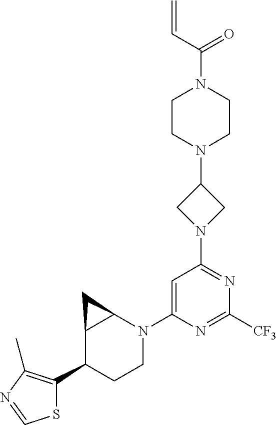 [SMILES: C=CC(=O)N1CCN(C2CN(c3cc(N4CC[C@@H](c5scnc5C)[C@H]5C[C@H]54)nc(C(F)(F)F)n3)C2)CC1] | 1-(4-(1-(6-((1R,5R,6R)-5-(4- methylthiazol-5-yl)-2- azabicyclo[4.1.0]heptan-2-yl)- 2-(trifluoromethyl)pyrimidin- 4-yl)azetidin-3-yl)piperazin-1- yl)prop-2-en-1-one | E-3 | 533.7 |
| 2-092 | 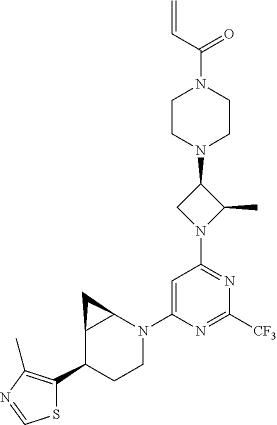 [SMILES: C=CC(=O)N1CCN([C@@H]2CN(c3cc(N4CC[C@@H](c5scnc5C)[C@H]5C[C@H]54)nc(C(F)(F)F)n3)[C@@H]2C)CC1] | 1-(4-((2R,3R)-2-methyl-1-(6- ((1R,5R,6R)-5-(4- methylthiazol-5-yl)-2- azabicyclo[4.1.0]heptan-2-yl)- 2-(trifluoromethyl)pyrimidin- 4-yl)azetidin-3-yl)piperazin-1- yl)prop-2-en-1-one | E-1 | 547.7 |
| 2-093 | 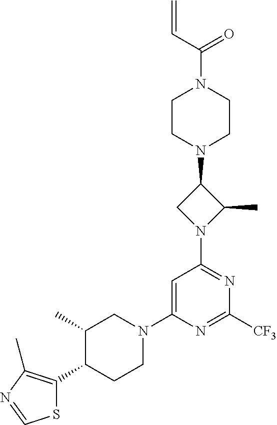 [SMILES: C=CC(=O)N1CCN([C@@H]2CN(c3cc(N4CC[C@H](c5scnc5C)[C@H](C)C4)nc(C(F)(F)F)n3)[C@@H]2C)CC1] | 1-(4-((2R,3R)-2-methyl-1-(6- ((3S,4S)-3-methyl-4-(4- methylthiazol-5-yl)piperidin- 1-yl)-2- (trifluoromethyl)pyrimidin-4- yl)azetidin-3-yl)piperazin-1- yl)prop-2-en-1-one | E-1 | 549.8 |
| 2-094 | 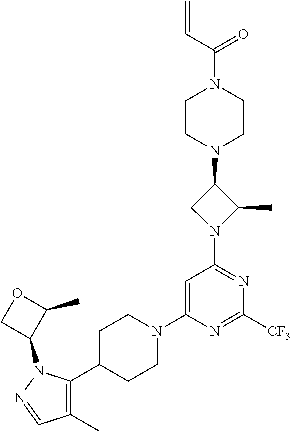 [SMILES: C=CC(=O)N1CCN([C@@H]2CN(c3cc(N4CCC(c5c(C)cnn5[C@H]5CO[C@H]5C)CC4)nc(C(F)(F)F)n3)[C@@H]2C)CC1] | 1-(4-((2R,3R)-2-methyl-1-(6- (4-(4-methyl-1-((2S,3S)-2- methyloxetan-3-yl)-1H- pyrazol-5-yl)piperidin-1-yl)-2- (trifluoromethyl)pyrimidin-4- yl)azetidin-3-yl)piperazin-1- yl)prop-2-en-1-one | E-8 | 589.3 |
| 2-095 | 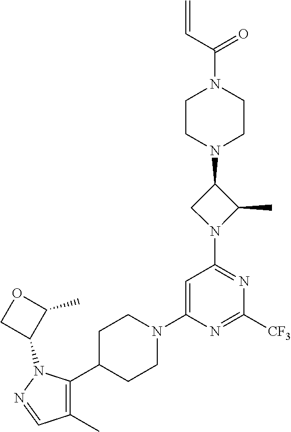 [SMILES: C=CC(=O)N1CCN([C@@H]2CN(c3cc(N4CCC(c5c(C)cnn5[C@@H]5CO[C@@H]5C)CC4)nc(C(F)(F)F)n3)[C@@H]2C)CC1] | 1-(4-((2R,3R)-2-methyl-1-(6- (4-(4-methyl-1-((2R,3R)-2- methyloxetan-3-yl)-1H- pyrazol-5-yl)piperidin-1-yl)-2- (trifluoromethyl)pyrimidin-4- yl)azetidin-3-yl)piperazin-1- yl)prop-2-en-1-one | E-8 | 589.3 |
| 2-096 | 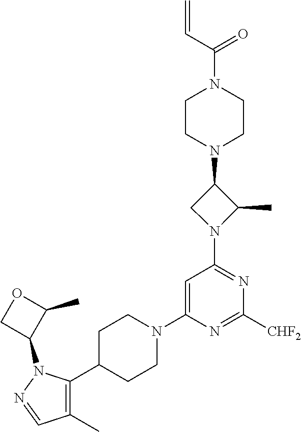 [SMILES: C=CC(=O)N1CCN([C@@H]2CN(c3cc(N4CCC(c5c(C)cnn5[C@H]5CO[C@H]5C)CC4)nc(C(F)F)n3)[C@@H]2C)CC1] | 1-(4-((2R,3R)-1-(2- (difluoromethyl)-6-(4-(4- methyl-1-((2S,3S)-2- methyloxetan-3-yl)-1H- pyrazol-5-yl)piperidin-1- yl)pyrimidin-4-yl)-2- methylazetidin-3-yl)piperazin- 1-yl)prop-2-en-1-one | E-2 | 571.3 |
| 2-097 | 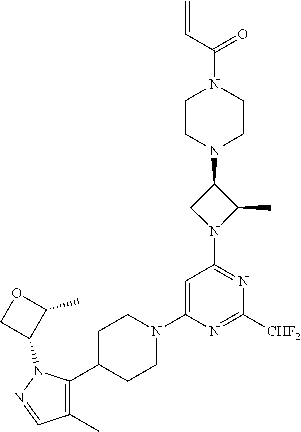 [SMILES: C=CC(=O)N1CCN([C@@H]2CN(c3cc(N4CCC(c5c(C)cnn5[C@@H]5CO[C@@H]5C)CC4)nc(C(F)F)n3)[C@@H]2C)CC1] | 1-(4-((2R,3R)-1-(2- (difluoromethyl)-6-(4-(4- methyl-1-((2R,3R)-2- methyloxetan-3-yl)-1H- pyrazol-5-yl)piperidin-1- yl)pyrimidin-4-yl)-2- methylazetidin-3-yl)piperazin- 1-yl)prop-2-en-1-one | E-2 | 571.3 |
| 2-098 | 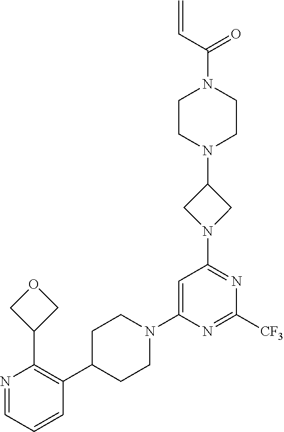 [SMILES: C=CC(=O)N1CCN(C2CN(c3cc(N4CCC(c5cccnc5C5COC5)CC4)nc(C(F)(F)F)n3)C2)CC1] | 1-(4-(1-(6-(4-(2-(oxetan-3- yl)pyridin-3-yl)piperidin-1- yl)-2- (trifluoromethyl)pyrimidin-4- yl)azetidin-3-yl)piperazin-1- yl)prop-2-en-1-one | E-1 | 558.3 |
| 2-099 | 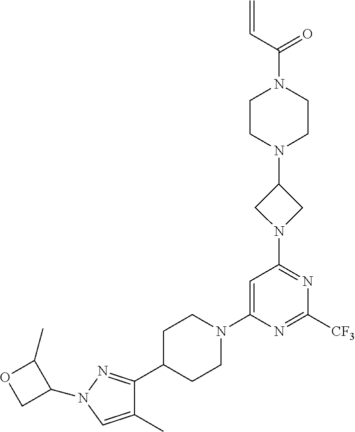 [SMILES: C=CC(=O)N1CCN(C2CN(c3cc(N4CCC(c5nn(C6COC6C)cc5C)CC4)nc(C(F)(F)F)n3)C2)CC1] | 1-(4-(1-(6-(4-(4-methyl-1-(2- methyloxetan-3-yl)-1H- pyrazol-3-yl)piperidin-1-yl)-2- (trifluoromethyl)pyrimidin-4- yl)azetidin-3-yl)piperazin-1- yl)prop-2-en-1-one | E-3 | 574.7 |
| 2-100 | 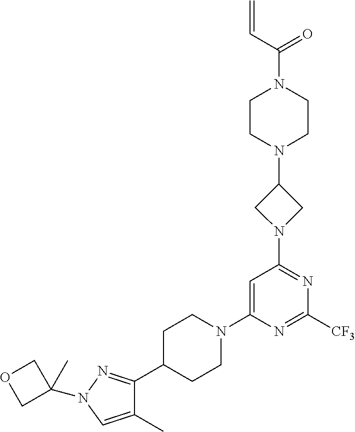 [SMILES: C=CC(=O)N1CCN(C2CN(c3cc(N4CCC(c5nn(C6(C)COC6)cc5C)CC4)nc(C(F)(F)F)n3)C2)CC1] | 1-(4-(1-(6-(4-(4-methyl-1-(3- methyloxetan-3-yl)-1H- pyrazol-3-yl)piperidin-1-yl)-2- (trifluoromethyl)pyrimidin-4- yl)azetidin-3-yl)piperazin-1- yl)prop-2-en-1-one | E-3 | 574.7 |
| 2-101 | 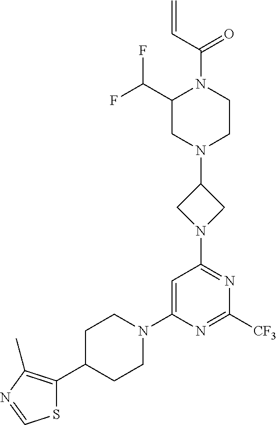 [SMILES: C=CC(=O)N1CCN(C2CN(c3cc(N4CCC(c5scnc5C)CC4)nc(C(F)(F)F)n3)C2)CC1C(F)F] | 1-(2-(difluoromethyl)-4-(1-(6- (4-(4-methylthiazol-5- yl)piperidin-1-yl)-2- (trifluoromethyl)pyrimidin-4- yl)azetidin-3-yl)piperazin-1- yl)prop-2-en-1-one | E-7 | 572.1 |
| 2-102 | 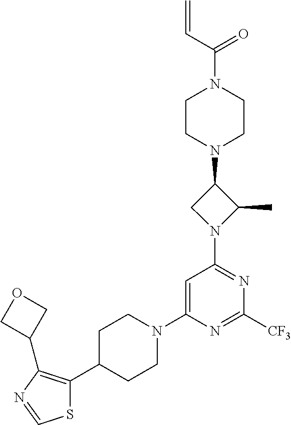 [SMILES: C=CC(=O)N1CCN([C@@H]2CN(c3cc(N4CCC(c5scnc5C5COC5)CC4)nc(C(F)(F)F)n3)[C@@H]2C)CC1] | 1-(4-((2R,3R)-2-methyl-1-(6- (4-(4-(oxetan-3-yl)thiazol-5- yl)piperidin-1-yl)-2- (trifluoromethyl)pyrimidin-4- yl)azetidin-3-yl)piperazin-1- yl)prop-2-en-1-one | E-1 | 578.2 |
| 2-390 | 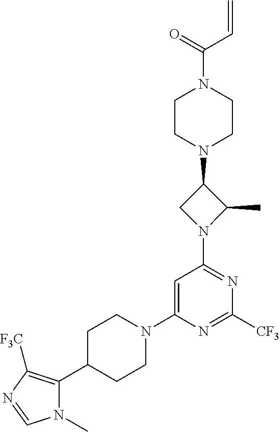 [SMILES: C=CC(=O)N1CCN([C@@H]2CN(c3cc(N4CCC(c5c(C(F)(F)F)ncn5C)CC4)nc(C(F)(F)F)n3)[C@@H]2C)CC1] | 1-(4-((2R,3R)-2-methyl-1-(6- (4-(1-methyl-4- (trifluoromethyl)-1H- imidazol-5-yl)-1-piperidinyl)- 2-(trifluoromethyl)-4- pyrimidinyl)-3-azetidinyl)-1- piperazinyl)-2-propen-1-one | E-1 | 587.3 |
| 2-391 | 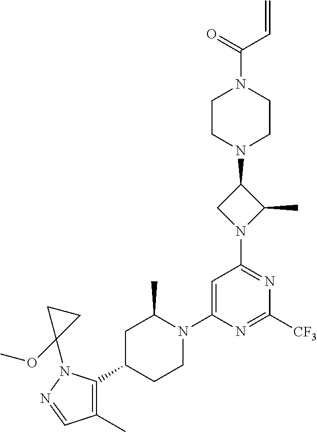 [SMILES: C=CC(=O)N1CCN([C@@H]2CN(c3cc(N4CC[C@H](c5c(C)cnn5C5(OC)CC5)C[C@H]4C)nc(C(F)(F)F)n3)[C@@H]2C)CC1] | 1-(4-((2R,3R)-1-(6-((2R,4S)- 4-(1-(1-methoxycyclopropyl)- 4-methyl-1H-pyrazol-5-yl)-2- methyl-1-piperidinyl)-2- (trifluoromethyl)-4- pyrimidinyl)-2-methyl-3- azetidinyl)-1-piperazinyl)-2- propen-1-one | E-1 | 603.0 |
| 2-393 | 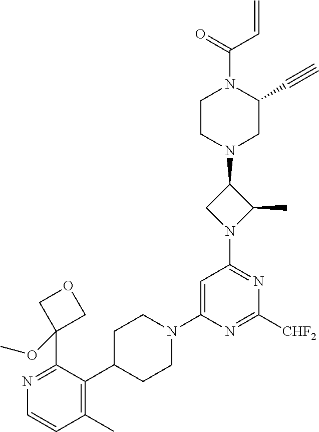 [SMILES: C#C[C@@H]1CN([C@@H]2CN(c3cc(N4CCC(c5c(C)ccnc5C5(OC)COC5)CC4)nc(C(F)F)n3)[C@@H]2C)CCN1C(=O)C=C] | 1-((2R)-4-((2R,3R)-1-(2- (difluoromethyl)-6-(4-(2-(3- methoxy-3-oxetanyl)-4- methyl-3-pyridinyl)-1- piperidinyl)-4-pyrimidinyl)-2- methyl-3-azetidinyl)-2- ethynyl-1-piperazinyl)-2- propen-1-one | E-2 | 622.0 |
| 2-394 | 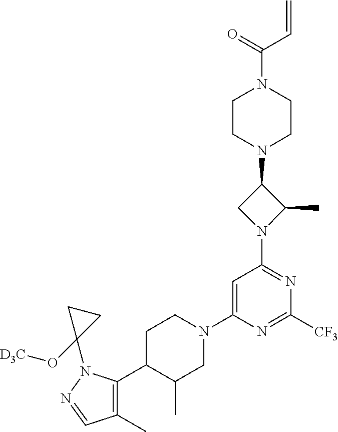 [SMILES: [2H]C([2H])([2H])OC1(n2ncc(C)c2C2CCN(c3cc(N4C[C@@H](N5CCN(C(=O)C=C)CC5)[C@H]4C)nc(C(F)(F)F)n3)CC2C)CC1] | 1-(4-((2R,3R)-1-(6-(4-(1-(1- (methoxy-d3)cyclopropyl)-4- methyl-1H-pyrazol-5-yl)-3- methylpiperidin-1-yl)-2- (trifluoromethyl)pyrimidin-4- yl)-2-methylazetidin-3- yl)piperazin-1-yl)prop-2-en-1- one | E-1 | 606.0 |
| 2-395 | 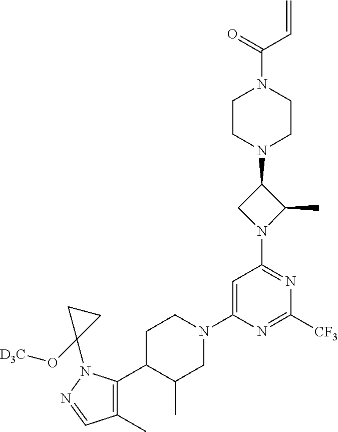 [SMILES: [2H]C([2H])([2H])OC1(n2ncc(C)c2C2CCN(c3cc(N4C[C@@H](N5CCN(C(=O)C=C)CC5)[C@H]4C)nc(C(F)(F)F)n3)CC2C)CC1] | 1-(4-((2R,3R)-1-(6-(4-(1-(1- (methoxy-d3)cyclopropyl)-4- methyl-1H-pyrazol-5-yl)-3- methylpiperidin-1-yl)-2- (trifluoromethyl)pyrimidin-4- yl)-2-methylazetidin-3- yl)piperazin-1-yl)prop-2-en-1- one | E-1 | 606.0 |
| 2-396 | 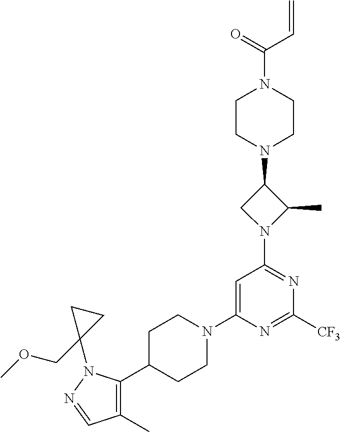 [SMILES: C=CC(=O)N1CCN([C@@H]2CN(c3cc(N4CCC(c5c(C)cnn5C5(COC)CC5)CC4)nc(C(F)(F)F)n3)[C@@H]2C)CC1] | 1-(4-((2R,3R)-1-(6-(4-(1-(1- (methoxymethyl)cyclopropyl)- 4-methyl-1H-pyrazol-5-yl)-1- piperidinyl)-2- (trifluoromethyl)-4- pyrimidinyl)-2-methyl-3- azetidinyl)-1-piperazinyl)-2- propen-1-one | E-1 | 603.0 |
| 2-397 | 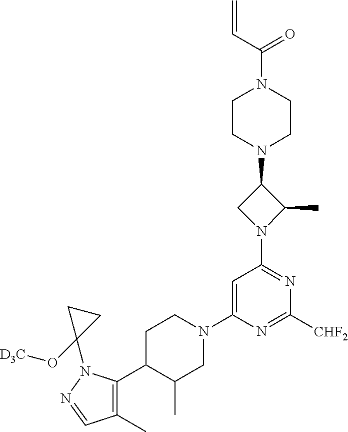 [SMILES: [2H]C([2H])([2H])OC1(n2ncc(C)c2C2CCN(c3cc(N4C[C@@H](N5CCN(C(=O)C=C)CC5)[C@H]4C)nc(C(F)F)n3)CC2C)CC1] | 1-(4-((2R,3R)-1-(2- (difluoromethyl)-6-(4-(1-(1- (methoxy-d3)cyclopropyl)-4- methyl-1H-pyrazol-5-yl)-3- methylpiperidin-1- yl)pyrimidin-4-yl)-2- methylazetidin-3-yl)piperazin- 1-yl)prop-2-en-1-one | E-2 | 588.0 |
| 2-398 | 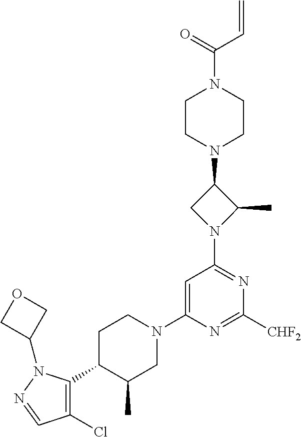 [SMILES: C=CC(=O)N1CCN([C@@H]2CN(c3cc(N4CC[C@@H](c5c(Cl)cnn5C5COC5)[C@H](C)C4)nc(C(F)F)n3)[C@@H]2C)CC1] | 1-(4-((2R,3R)-1-(6-((3S,4R)- 4-(4-chloro-1-(3-oxetanyl)- 1H-pyrazol-5-yl)-3-methyl-1- piperidinyl)-2- (difluoromethyl)-4- pyrimidinyl)-2-methyl-3- azetidinyl)-1-piperazinyl)-2- propen-1-one | E-2 | 591.0 |
| 2-400 | 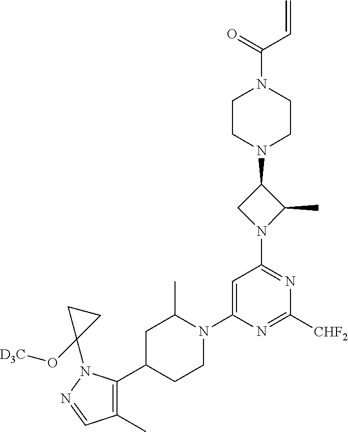 [SMILES: [2H]C([2H])([2H])OC1(n2ncc(C)c2C2CCN(c3cc(N4C[C@@H](N5CCN(C(=O)C=C)CC5)[C@H]4C)nc(C(F)F)n3)C(C)C2)CC1] | 1-(4-((2R,3R)-1-(2- (difluoromethyl)-6-(4-(1-(1- (methoxy-d3)cyclopropyl)-4- methyl-1H-pyrazol-5-yl)-2- methylpiperidin-1- yl)pyrimidin-4-yl)-2- methylazetidin-3-yl)piperazin- 1-yl)prop-2-en-1-one | E-2 | 588.0 |
| 2-401 | 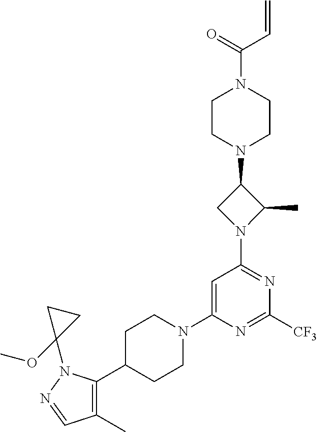 [SMILES: C=CC(=O)N1CCN([C@@H]2CN(c3cc(N4CCC(c5c(C)cnn5C5(OC)CC5)CC4)nc(C(F)(F)F)n3)[C@@H]2C)CC1] | 1-(4-((2R,3R)-1-(6-(4-(1-(1- methoxycyclopropyl)-4- methyl-1H-pyrazol-5-yl)-1- piperidinyl)-2- (trifluoromethyl)-4- pyrimidinyl)-2-methyl-3- azetidinyl)-1-piperazinyl)-2- propen-1-one | E-1 | 589.0 |
| 2-402 | 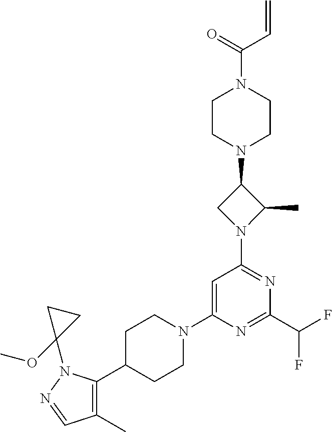 [SMILES: C=CC(=O)N1CCN([C@@H]2CN(c3cc(N4CCC(c5c(C)cnn5C5(OC)CC5)CC4)nc(C(F)F)n3)[C@@H]2C)CC1] | 1-(4-((2R,3R)-1-(2- (difluoromethyl)-6-(4-(1-(1- methoxycyclopropyl)-4- methyl-1H-pyrazol-5-yl)-1- piperidinyl)-4-pyrimidinyl)-2- methyl-3-azetidinyl)-1- piperazinyl)-2-propen-1-one | E-2 | 571.0 |
| 2-403 | 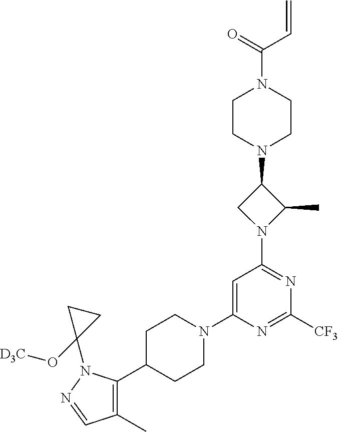 [SMILES: [2H]C([2H])([2H])OC1(n2ncc(C)c2C2CCN(c3cc(N4C[C@@H](N5CCN(C(=O)C=C)CC5)[C@H]4C)nc(C(F)(F)F)n3)CC2)CC1] | 1-(4-((2R,3R)-2-methyl-1-(6- (4-(1-(1-(methoxy- d3)cyclopropyl)-4-methyl-1H- pyrazol-5-yl)-1-piperidinyl)-2- (trifluoromethyl)-4- pyrimidinyl)-3-azetidinyl)-1- piperazinyl)-2-propen-1-one | E-1 | 592.0 |
| 2-404 | 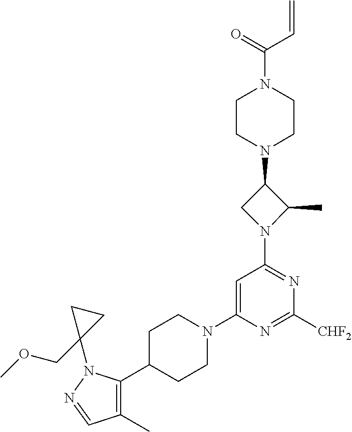 [SMILES: C=CC(=O)N1CCN([C@@H]2CN(c3cc(N4CCC(c5c(C)cnn5C5(COC)CC5)CC4)nc(C(F)F)n3)[C@@H]2C)CC1] | 1-(4-((2R,3R)-1-(2- (difluoromethyl)-6-(4-(1-(1- (methoxymethyl)cyclopropyl)- 4-methyl-1H-pyrazol-5-yl)-1- piperidinyl)-4-pyrimidinyl)-2- methyl-3-azetidinyl)-1- piperazinyl)-2-propen-1-one | E-2 | 585.0 |
| 2-405 | 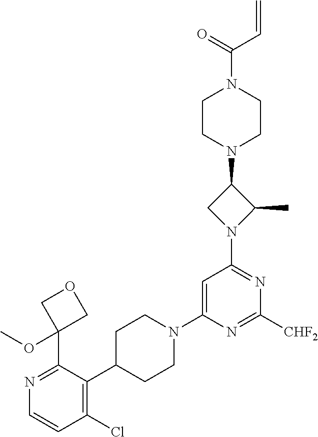 [SMILES: C=CC(=O)N1CCN([C@@H]2CN(c3cc(N4CCC(c5c(Cl)ccnc5C5(OC)COC5)CC4)nc(C(F)F)n3)[C@@H]2C)CC1] | 1-(4-((2R,3R)-1-(6-(4-(4- chloro-2-(3-methoxy-3- oxetanyl)-3-pyridinyl)-1- piperidinyl)-2- (difluoromethyl)-4- pyrimidinyl)-2-methyl-3- azetidinyl)-1-piperazinyl)-2- propen-1-one | E-2 | 618.0 |
| 2-406 | 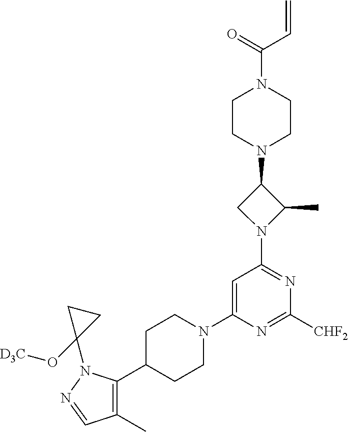 [SMILES: [2H]C([2H])([2H])OC1(n2ncc(C)c2C2CCN(c3cc(N4C[C@@H](N5CCN(C(=O)C=C)CC5)[C@H]4C)nc(C(F)F)n3)CC2)CC1] | 1-(4-((2R,3R)-1-(2- (difluoromethyl)-6-(4-(1-(1- (methoxy-d3)cyclopropyl)-4- methyl-1H-pyrazol-5- yl)piperidin-1-yl)pyrimidin-4- yl)-2-methylazetidin-3- yl)piperazin-1-yl)prop-2-en-1- one | E-2 | 574.0 |
| 2-408 | 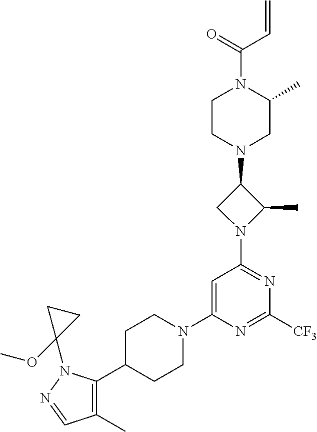 [SMILES: C=CC(=O)N1CCN([C@@H]2CN(c3cc(N4CCC(c5c(C)cnn5C5(OC)CC5)CC4)nc(C(F)(F)F)n3)[C@@H]2C)C[C@H]1C] | 1-((2R)-4-((2R,3R)-1-(6-(4- (1-(1-methoxycyclopropyl)-4- methyl-1H-pyrazol-5-yl)-1- piperidinyl)-2- (trifluoromethyl)-4- pyrimidinyl)-2-methyl-3- azetidinyl)-2-methyl-1- piperazinyl)-2-propen-1-one | E-1 | 603.2 |
| 2-409 | 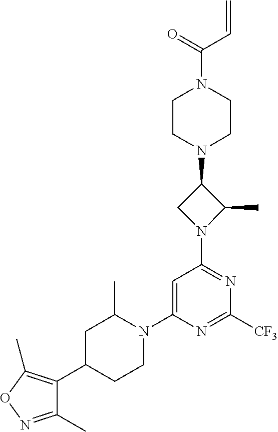 [SMILES: C=CC(=O)N1CCN([C@@H]2CN(c3cc(N4CCC(c5c(C)noc5C)CC4C)nc(C(F)(F)F)n3)[C@@H]2C)CC1] | 1-(4-((2R,3R)-1-(6-(4-(3,5- dimethyl-1,2-oxazol-4-yl)-2- methyl-1-piperidinyl)-2- (trifluoromethyl)-4- pyrimidinyl)-2-methyl-3- azetidinyl)-1-piperazinyl)-2- propen-1-one | E-1 | 547.9 |
| 2-410 | 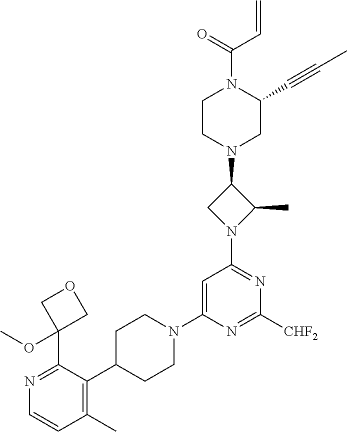 [SMILES: C=CC(=O)N1CCN([C@@H]2CN(c3cc(N4CCC(c5c(C)ccnc5C5(OC)COC5)CC4)nc(C(F)F)n3)[C@@H]2C)C[C@H]1C#CC] | 1-((2R)-4-((2R,3R)-1-(2- (difluoromethyl)-6-(4-(2-(3- methoxy-3-oxetanyl)-4- methyl-3-pyridinyl)-1- piperidinyl)-4-pyrimidinyl)-2- methyl-3-azetidinyl)-2-(1- propyn-1-yl)-1-piperazinyl)-2- propen-1-one | E-2 | 636.0 |
| 2-413 | 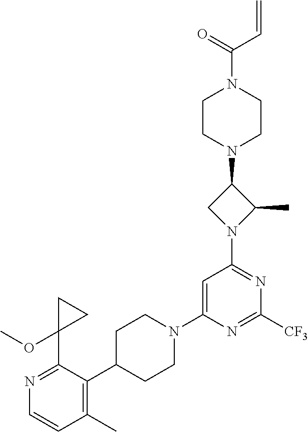 [SMILES: C=CC(=O)N1CCN([C@@H]2CN(c3cc(N4CCC(c5c(C)ccnc5C5(OC)CC5)CC4)nc(C(F)(F)F)n3)[C@@H]2C)CC1] | 1-(4-((2R,3R)-1-(6-(4-(2-(1- methoxycyclopropyl)-4- methyl-3-pyridinyl)-1- piperidinyl)-2- (trifluoromethyl)-4- pyrimidinyl)-2-methyl-3- azetidinyl)-1-piperazinyl)-2- propen-1-one | E-1 | 600.0 |
| 2-414 | 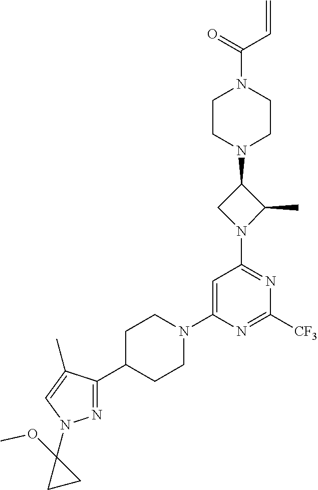 [SMILES: C=CC(=O)N1CCN([C@@H]2CN(c3cc(N4CCC(c5nn(C6(OC)CC6)cc5C)CC4)nc(C(F)(F)F)n3)[C@@H]2C)CC1] | 1-(4-((2R,3R)-1-(6-(4-(1-(1- methoxycyclopropyl)-4- methyl-1H-pyrazol-3-yl)-1- piperidinyl)-2- (trifluoromethyl)-4- pyrimidinyl)-2-methyl-3- azetidinyl)-1-piperazinyl)-2- propen-1-one | E-8 | 589.0 |
| 2-417 | 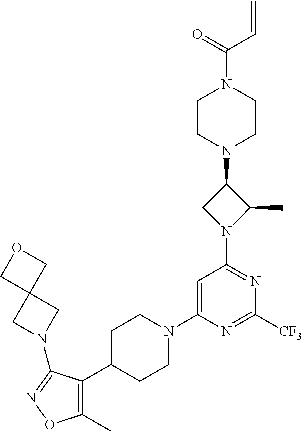 [SMILES: C=CC(=O)N1CCN([C@@H]2CN(c3cc(N4CCC(c5c(N6CC7(COC7)C6)noc5C)CC4)nc(C(F)(F)F)n3)[C@@H]2C)CC1] | 1-(4-((2R,3R)-2-methyl-1-(6- (4-(5-methyl-3-(2-oxa-6- azaspiro[3.3]heptan-6-yl)-1,2- oxazol-4-yl)-1-piperidinyl)-2- (trifluoromethyl)-4- pyrimidinyl)-3-azetidinyl)-1- piperazinyl)-2-propen-1-one | E-1 | 617.0 |
| 2-421 | 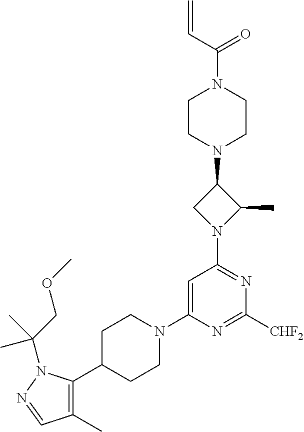 [SMILES: C=CC(=O)N1CCN([C@@H]2CN(c3cc(N4CCC(c5c(C)cnn5C(C)(C)COC)CC4)nc(C(F)F)n3)[C@@H]2C)CC1] | 1-(4-((2R,3R)-1-(2- (difluoromethyl)-6-(4-(1-(1- methoxy-2-methyl-2- propanyl)-4-methyl-1H- pyrazol-5-yl)-1-piperidinyl)-4- pyrimidinyl)-2-methyl-3- azetidinyl)-1-piperazinyl)-2- propen-1-one | E-2 | 587.3 |
| 2-422 | 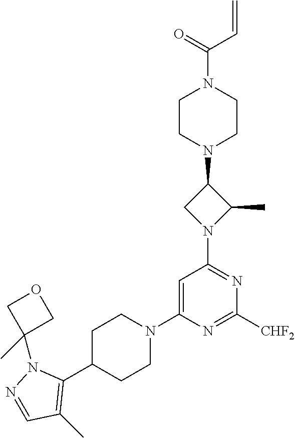 [SMILES: C=CC(=O)N1CCN([C@@H]2CN(c3cc(N4CCC(c5c(C)cnn5C5(C)COC5)CC4)nc(C(F)F)n3)[C@@H]2C)CC1] | 1-(4-((2R,3R)-1-(2- (difluoromethyl)-6-(4-(4- methyl-1-(3-methyl-3- oxetanyl)-1H-pyrazol-5-yl)-1- piperidinyl)-4-pyrimidinyl)-2- methyl-3-azetidinyl)-1- piperazinyl)-2-propen-1-one | E-9 | 571.2 |
| 2-423 | 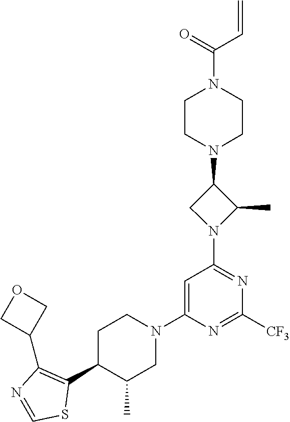 [SMILES: C=CC(=O)N1CCN([C@@H]2CN(c3cc(N4CC[C@H](c5scnc5C5COC5)[C@@H](C)C4)nc(C(F)(F)F)n3)[C@@H]2C)CC1] | 1-(4-((2R,3R)-2-methyl-1-(6- ((3R,4S)-3-methyl-4-(4-(3- oxetanyl)-1,3-thiazol-5-yl)-1- piperidinyl)-2- (trifluoromethyl)-4- pyrimidinyl)-3-azetidinyl)-1- piperazinyl)-2-propen-1-one | E-1 | 592.4 |
| 2-424 | 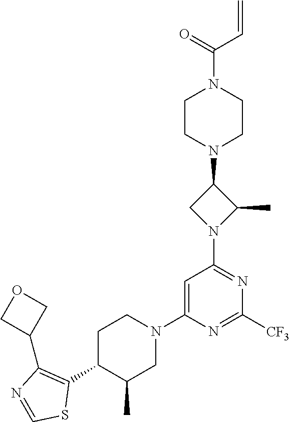 [SMILES: C=CC(=O)N1CCN([C@@H]2CN(c3cc(N4CC[C@@H](c5scnc5C5COC5)[C@H](C)C4)nc(C(F)(F)F)n3)[C@@H]2C)CC1] | 1-(4-((2R,3R)-2-methyl-1-(6- ((3S,4R)-3-methyl-4-(4-(3- oxetanyl)-1,3-thiazol-5-yl)-1- piperidinyl)-2- (trifluoromethyl)-4- pyrimidinyl)-3-azetidinyl)-1- piperazinyl)-2-propen-1-one | E-1 | 592.4 |
| 2-426 | 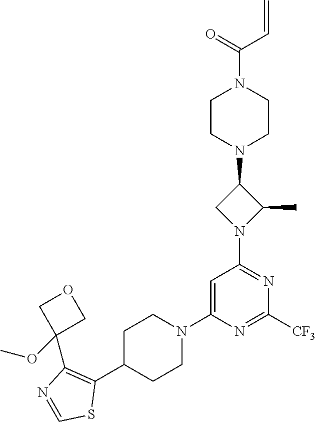 [SMILES: C=CC(=O)N1CCN([C@@H]2CN(c3cc(N4CCC(c5scnc5C5(OC)COC5)CC4)nc(C(F)(F)F)n3)[C@@H]2C)CC1] | 1-(4-((2R,3R)-1-(6-(4-(4-(3- methoxyoxetan-3-yl)thiazol-5- yl)piperidin-1-yl)-2- (trifluoromethyl)pyrimidin-4- yl)-2-methylazetidin-3- yl)piperazin-1-yl)prop-2-en-1- one | E-1 | 608.4 |
| 2-427 | 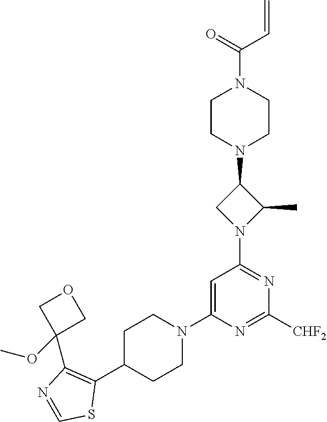 [SMILES: C=CC(=O)N1CCN([C@@H]2CN(c3cc(N4CCC(c5scnc5C5(OC)COC5)CC4)nc(C(F)F)n3)[C@@H]2C)CC1] | 1-(4-((2R,3R)-1-(2- (difluoromethyl)-6-(4-(4-(3- methoxy-3-oxetanyl)-1,3- thiazol-5-yl)-1-piperidinyl)-4- pyrimidinyl)-2-methyl-3- azetidinyl)-1-piperazinyl)-2- propen-1-one | E-2 | 590.4 |
| 2-428 | 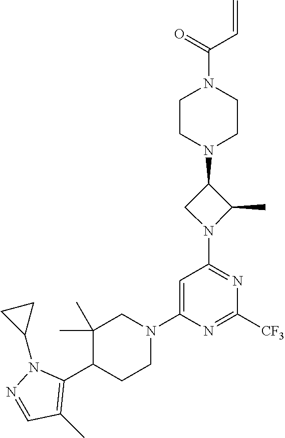 [SMILES: C=CC(=O)N1CCN([C@@H]2CN(c3cc(N4CCC(c5c(C)cnn5C5CC5)C(C)(C)C4)nc(C(F)(F)F)n3)[C@@H]2C)CC1] | 1-(4-((2R,3R)-1-(6-(4-(1- cyclopropyl-4-methyl-1H- pyrazol-5-yl)-3,3- dimethylpiperidin-1-yl)-2- (trifluoromethyl)pyrimidin-4- yl)-2-methylazetidin-3- yl)piperazin-1-yl)prop-2-en-1- one | E-1 | 587.5 |
| 2-430 | 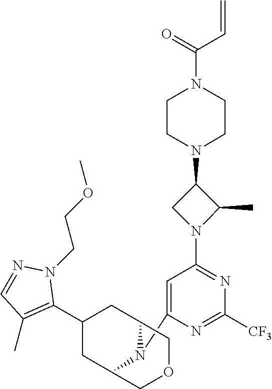 [SMILES: C=CC(=O)N1CCN([C@@H]2CN(c3cc(N4[C@@H]5COC[C@H]4CC(c4c(C)cnn4CCOC)C5)nc(C(F)(F)F)n3)[C@@H]2C)CC1] | 1-(4-((2R,3R)-1-(6-((7-endo)- 7-(1-(2-methoxyethyl)-4- methyl-1H-pyrazol-5-yl)-3- oxa-9-azabicyclo[3.3.1]nonan- 9-yl)-2-(trifluoromethyl)-4- pyrimidinyl)-2-methyl-3- azetidinyl)-1-piperazinyl)-2- propen-1-one | E-8 | 619.0 |
| 2-432 | 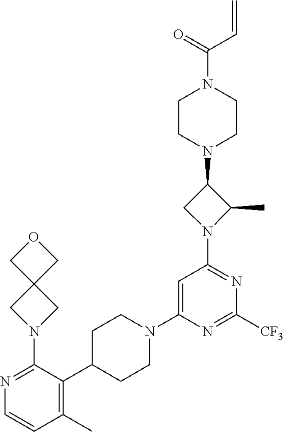 [SMILES: C=CC(=O)N1CCN([C@@H]2CN(c3cc(N4CCC(c5c(C)ccnc5N5CC6(COC6)C5)CC4)nc(C(F)(F)F)n3)[C@@H]2C)CC1] | 1-(4-((2R,3R)-2-methyl-1-(6- (4-(4-methyl-2-(2-oxa-6- azaspiro[3.3]heptan-6-yl)-3- pyridinyl)-1-piperidinyl)-2- (trifluoromethyl)-4- pyrimidinyl)-3-azetidinyl)-1- piperazinyl)-2-propen-1-one | E-8 | 626.9 |
| 2-433 | 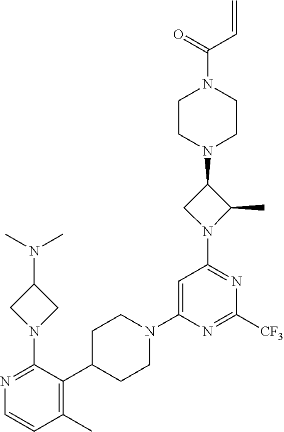 [SMILES: C=CC(=O)N1CCN([C@@H]2CN(c3cc(N4CCC(c5c(C)ccnc5N5CC(N(C)C)C5)CC4)nc(C(F)(F)F)n3)[C@@H]2C)CC1] | 1-(4-((2R,3R)-1-(6-(4-(2-(3- (dimethylamino)-1- azetidinyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-8 | 628.0 |
| 2-434 | 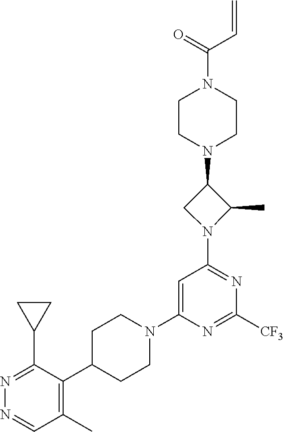 [SMILES: C=CC(=O)N1CCN([C@@H]2CN(c3cc(N4CCC(c5c(C)cnnc5C5CC5)CC4)nc(C(F)(F)F)n3)[C@@H]2C)CC1] | 1-(4-((2R,3R)-1-(6-(4-(3- cyclopropyl-5-methyl-4- pyridazinyl)-1-piperidinyl)-2- (trifluoromethyl)-4- pyrimidinyl)-2-methyl-3- azetidinyl)-1-piperazinyl)-2- propen-1-one | E-8 | 571.0 |
| 2-435 | 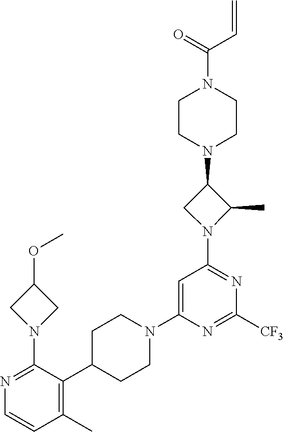 [SMILES: C=CC(=O)N1CCN([C@@H]2CN(c3cc(N4CCC(c5c(C)ccnc5N5CC(OC)C5)CC4)nc(C(F)(F)F)n3)[C@@H]2C)CC1] | 1-(4-((2R,3R)-1-(6-(4-(2-(3- methoxy-1-azetidinyl)-4- methyl-3-pyridinyl)-1- piperidinyl)-2- (trifluoromethyl)-4- pyrimidinyl)-2-methyl-3- azetidinyl)-1-piperazinyl)-2- propen-1-one | E-8 | 614.9 |
| 2-436 | 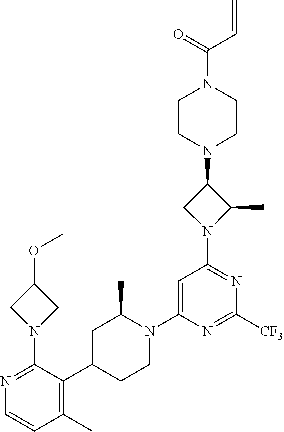 [SMILES: C=CC(=O)N1CCN([C@@H]2CN(c3cc(N4CCC(c5c(C)ccnc5N5CC(OC)C5)C[C@H]4C)nc(C(F)(F)F)n3)[C@@H]2C)CC1] | 1-(4-((2R,3R)-1-(6-((2R)-4- (2-(3-methoxy-1-azetidinyl)- 4-methyl-3-pyridinyl)-2- methyl-1-piperidinyl)-2- (trifluoromethyl)-4- pyrimidinyl)-2-methyl-3- azetidinyl)-1-piperazinyl)-2- propen-1-one | E-8 | 628.9 |
| | mixture of cis/trans isomers | | | |
| 2-437 | 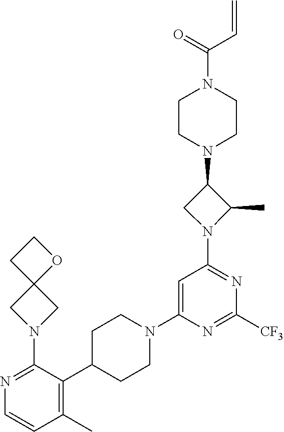 [SMILES: C=CC(=O)N1CCN([C@@H]2CN(c3cc(N4CCC(c5c(C)ccnc5N5CC6(CCO6)C5)CC4)nc(C(F)(F)F)n3)[C@@H]2C)CC1] | 1-(4-((2R,3R)-2-methyl-1-(6- (4-(4-methyl-2-(1-oxa-6- azaspiro[3.3]heptan-6-yl)-3- pyridinyl)-1-piperidinyl)-2- (trifluoromethyl)-4- pyrimidinyl)-3-azetidinyl)-1- piperazinyl)-2-propen-1-one | E-8 | 627.0 |
| 2-438 | 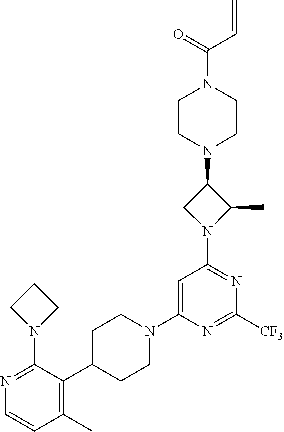 [SMILES: C=CC(=O)N1CCN([C@@H]2CN(c3cc(N4CCC(c5c(C)ccnc5N5CCC5)CC4)nc(C(F)(F)F)n3)[C@@H]2C)CC1] | 1-(4-((2R,3R)-1-(6-(4-(2- (azetidin-1-yl)-4- methylpyridin-3-yl)piperidin- 1-yl)-2- (trifluoromethyl)pyrimidin-4- yl)-2-methylazetidin-3- yl)piperazin-1-yl)prop-2-en-1- one | E-8 | 585.0 |
| 2-439 | 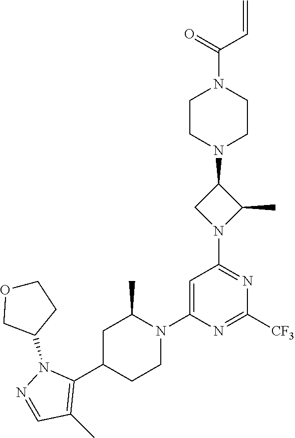 | 1-(4-((2R,3R)-2-methyl-1-(6- ((2R,4S)-2-methyl-4-(4- methyl-1-((S)- tetrahydrofuran-3-yl)-1H- pyrazol-5-yl)piperidin-1-yl)-2- (trifluoromethyl)pyrimidin-4- yl)azetidin-3-yl)piperazin-1- yl)prop-2-en-1-one | E-8 | 603.0 |
| 2-440 | 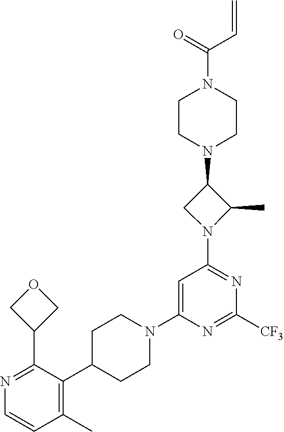 [SMILES: C=CC(=O)N1CCN([C@@H]2CN(c3cc(N4CCC(c5c(C)ccnc5C5COC5)CC4)nc(C(F)(F)F)n3)[C@@H]2C)CC1] | 1-(4-((2R,3R)-2-methyl-1-(6- (4-(4-methyl-2-(3-oxetanyl)- 3-pyridinyl)-1-piperidinyl)-2- (trifluoromethyl)-4- pyrimidinyl)-3-azetidinyl)-1- piperazinyl)-2-propen-1-one | E-8 | 586.0 |
| 2-443 | 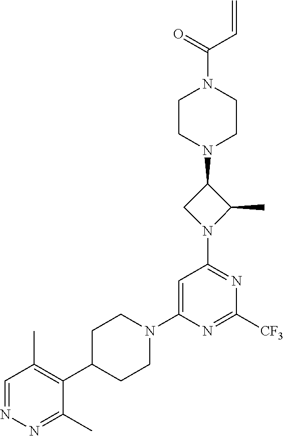 [SMILES: C=CC(=O)N1CCN([C@@H]2CN(c3cc(N4CCC(c5c(C)cnnc5C)CC4)nc(C(F)(F)F)n3)[C@@H]2C)CC1] | 1-(4-((2R,3R)-1-(6-(4-(3,5- dimethyl-4-pyridazinyl)-1- piperidinyl)-2- (trifluoromethyl)-4- pyrimidinyl)-2-methyl-3- azetidinyl)-1-piperazinyl)-2- propen-1-one | E-8 | 545.2 |
| 2-445 | 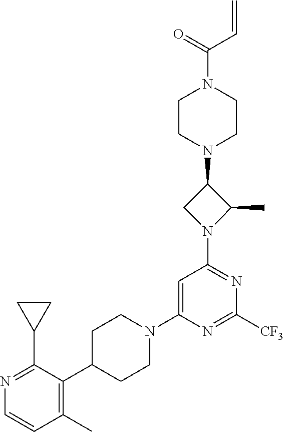 [SMILES: C=CC(=O)N1CCN([C@@H]2CN(c3cc(N4CCC(c5c(C)ccnc5C5CC5)CC4)nc(C(F)(F)F)n3)[C@@H]2C)CC1] | 1-(4-((2R,3R)-1-(6-(4-(2- cyclopropyl-4-methyl-3- pyridinyl)-1-piperidinyl)-2- (trifluoromethyl)-4- pyrimidinyl)-2-methyl-3- azetidinyl)-1-piperazinyl)-2- propen-1-one | E-8 | 570.1 |
| 2-446 | 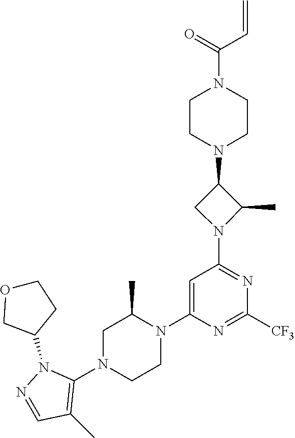 | 1-(4-((2R,3R)-2-methyl-1-(6- ((R)-2-methyl-4-(4-methyl-1- ((S)-tetrahydrofuran-3-yl)-1H- pyrazol-5-yl)piperazin-1-yl)- 2-(trifluoromethyl)pyrimidin- 4-yl)azetidin-3-yl)piperazin-1- yl)prop-2-en-1-one | E-8 | 604.0 |
| 2-447 | 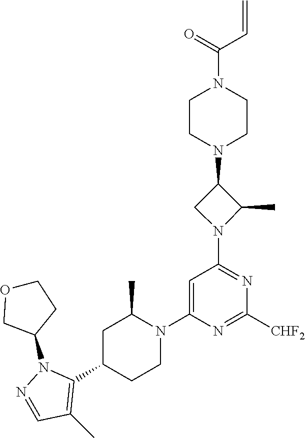 [SMILES: C=CC(=O)N1CCN([C@@H]2CN(c3cc(N4CC[C@H](c5c(C)cnn5[C@@H]5CCOC5)C[C@H]4C)nc(C(F)F)n3)[C@@H]2C)CC1] | 1-(4-((2R,3R)-1-(2- (difluoromethyl)-6-((2R,4S)- 2-methyl-4-(4-methyl-1-((R)- tetrahydrofuran-3-yl)-1H- pyrazol-5-yl)piperidin-1- yl)pyrimidin-4-yl)-2- methylazetidin-3-yl)piperazin- 1-yl)prop-2-en-1-one | E-9 | 585.0 |
| 2-450 | 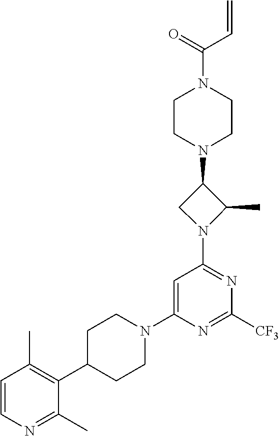 [SMILES: C=CC(=O)N1CCN([C@@H]2CN(c3cc(N4CCC(c5c(C)ccnc5C)CC4)nc(C(F)(F)F)n3)[C@@H]2C)CC1] | 1-(4-((2R,3R)-1-(6-(4-(2,4- dimethyl-3-pyridinyl)-1- piperidinyl)-2- (trifluoromethyl)-4- pyrimidinyl)-2-methyl-3- azetidinyl)-1-piperazinyl)-2- propen-1-one | E-8 | 544.0 |
| 2-451 | 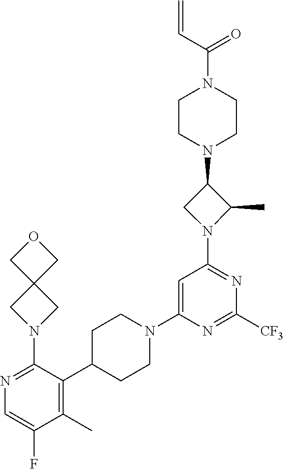 [SMILES: C=CC(=O)N1CCN([C@@H]2CN(c3cc(N4CCC(c5c(N6CC7(COC7)C6)ncc(F)c5C)CC4)nc(C(F)(F)F)n3)[C@@H]2C)CC1] | 1-(4-((2R,3R)-1-(6-(4-(5- fluoro-4-methyl-2-(2-oxa-6- azaspiro[3.3]heptan-6-yl)-3- pyridinyl)-1-piperidinyl)-2- (trifluoromethyl)-4- pyrimidinyl)-2-methyl-3- azetidinyl)-1-piperazinyl)-2- propen-1-one | E-8 | 645.0 |
| 2-454 | 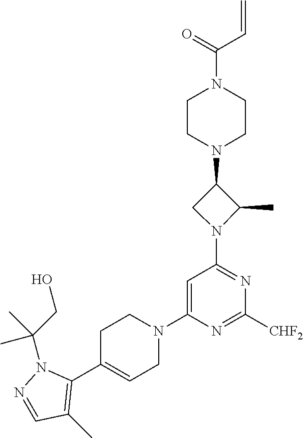 [SMILES: C=CC(=O)N1CCN([C@@H]2CN(c3cc(N4CC=C(c5c(C)cnn5C(C)(C)CO)CC4)nc(C(F)F)n3)[C@@H]2C)CC1] | 1-(4-((2R,3R)-1-(2- (difluoromethyl)-6-(4-(1-(1- hydroxy-2-methyl-2- propanyl)-4-methyl-1H- pyrazol-5-yl)-3,6-dihydro- 1(2H)-pyridinyl)-4- pyrimidinyl)-2-methyl-3- azetidinyl)-1-piperazinyl)-2- propen-1-one | E-2 | 571.0 |
| 2-455 | 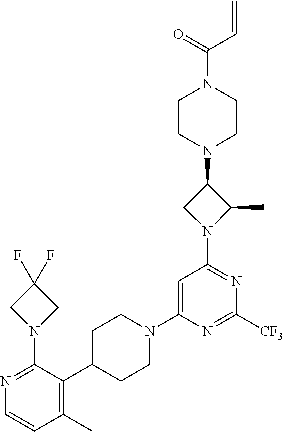 [SMILES: C=CC(=O)N1CCN([C@@H]2CN(c3cc(N4CCC(c5c(C)ccnc5N5CC(F)(F)C5)CC4)nc(C(F)(F)F)n3)[C@@H]2C)CC1] | 1-(4-((2R,3R)-1-(6-(4-(2-(3,3- difluoro-1-azetidinyl)-4- methyl-3-pyridinyl)-1- piperidinyl)-2- (trifluoromethyl)-4- pyrimidinyl)-2-methyl-3- azetidinyl)-1-piperazinyl)-2- propen-1-one | E-8 | 622.0 |
| 2-457 | 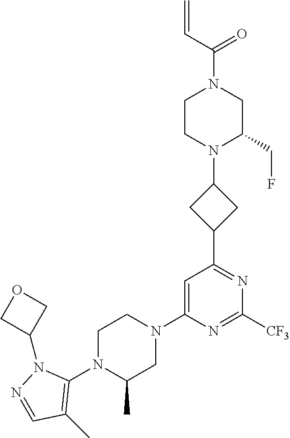 [SMILES: C=CC(=O)N1CCN(C2CC(c3cc(N4CCN(c5c(C)cnn5C5COC5)[C@H](C)C4)nc(C(F)(F)F)n3)C2)[C@@H](CF)C1] | 1-((R)-3-(fluoromethyl)-4-(3- (6-((R)-3-methyl-4-(4-methyl- 1-(oxetan-3-yl)-1H-pyrazol-5- yl)piperazin-1-yl)-2- (trifluoromethyl)pyrimidin-4- yl)cyclobutyl)piperazin-1- yl)prop-2-en-1-one | E-13 | 606.9 |
| | mixture of trans isomers | | | |
| 2-459 | 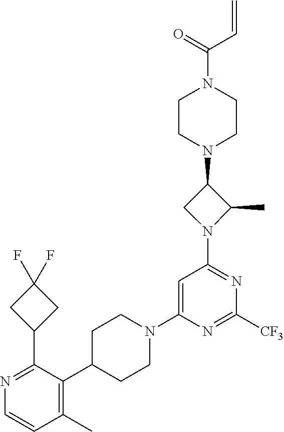 [SMILES: C=CC(=O)N1CCN([C@@H]2CN(c3cc(N4CCC(c5c(C)ccnc5C5CC(F)(F)C5)CC4)nc(C(F)(F)F)n3)[C@@H]2C)CC1] | 1-(4-((2R,3R)-1-(6-(4-(2-(3,3- difluorocyclobutyl)-4-methyl- 3-pyridinyl)-1-piperidinyl)-2- (trifluoromethyl)-4- pyrimidinyl)-2-methyl-3- azetidinyl)-1-piperazinyl)-2- propen-1-one | E-8 | 619.9 |
| 2-462 | 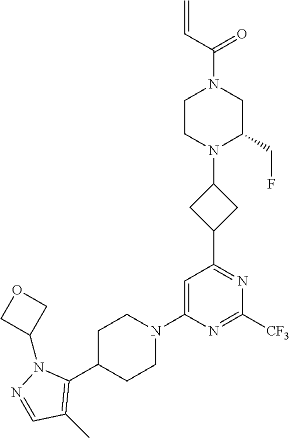 [SMILES: C=CC(=O)N1CCN(C2CC(c3cc(N4CCC(c5c(C)cnn5C5COC5)CC4)nc(C(F)(F)F)n3)C2)[C@@H](CF)C1] | (R)-1-(3-(fluoromethyl)-4-(3- (6-(4-(4-methyl-1-(oxetan-3- yl)-1H-pyrazol-5-yl)piperidin- 1-yl)-2- (trifluoromethyl)pyrimidin-4- yl)cyclobutyl)piperazin-1- yl)prop-2-en-1-one | E-13 | 592.0 |
| | mixture of trans isomers | | | |
| 2-463 | 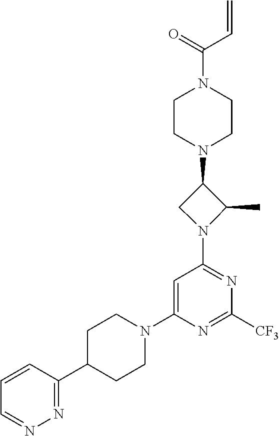 [SMILES: C=CC(=O)N1CCN([C@@H]2CN(c3cc(N4CCC(c5cccnn5)CC4)nc(C(F)(F)F)n3)[C@@H]2C)CC1] | 1-(4-((2R,3R)-2-methyl-1-(6- (4-(3-pyridazinyl)-1- piperidinyl)-2- (trifluoromethyl)-4- pyrimidinyl)-3-azetidinyl)-1- piperazinyl)-2-propen-1-one | E-8 | 517.0 |
| 2-464 | 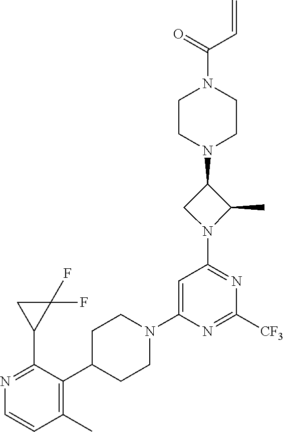 [SMILES: C=CC(=O)N1CCN([C@@H]2CN(c3cc(N4CCC(c5c(C)ccnc5C5CC5(F)F)CC4)nc(C(F)(F)F)n3)[C@@H]2C)CC1] | 1-(4-((2R,3R)-1-(6-(4-(2-(2,2- difluorocyclopropyl)-4- methyl-3-pyridinyl)-1- piperidinyl)-2- (trifluoromethyl)-4- pyrimidinyl)-2-methyl-3- azetidinyl)-1-piperazinyl)-2- propen-1-one | E-8 | 606.0 |
| 2-466 | 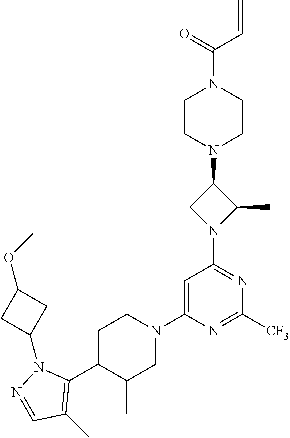 [SMILES: C=CC(=O)N1CCN([C@@H]2CN(c3cc(N4CCC(c5c(C)cnn5C5CC(OC)C5)C(C)C4)nc(C(F)(F)F)n3)[C@@H]2C)CC1] | 1-(4-((2R,3R)-1-(6-(4-(1-(3- methoxycyclobutyl)-4-methyl- 1H-pyrazol-5-yl)-3- methylpiperidin-1-yl)-2- (trifluoromethyl)pyrimidin-4- yl)-2-methylazetidin-3- yl)piperazin-1-yl)prop-2-en-1- one | E-8 | 617.2 |
| 2-469 | 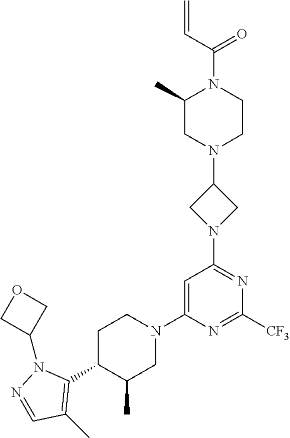 [SMILES: C=CC(=O)N1CCN(C2CN(c3cc(N4CC[C@@H](c5c(C)cnn5C5COC5)[C@H](C)C4)nc(C(F)(F)F)n3)C2)C[C@H]1C] | 1-((2R)-2-methyl-4-(1-(6- ((3S,4R)-3-methyl-4-(4- methyl-1-(3-oxetanyl)-1H- pyrazol-5-yl)-1-piperidinyl)-2- (trifluoromethyl)-4- pyrimidinyl)-3-azetidinyl)-1- piperazinyl)-2-propen-1-one | E-4 | 589.0 |
| 2-470 | 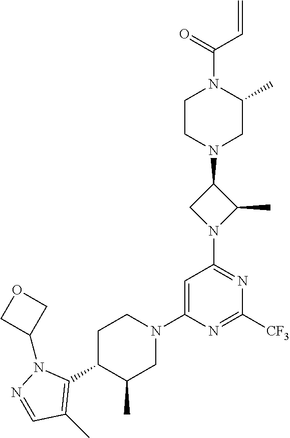 [SMILES: C=CC(=O)N1CCN([C@@H]2CN(c3cc(N4CC[C@@H](c5c(C)cnn5C5COC5)[C@H](C)C4)nc(C(F)(F)F)n3)[C@@H]2C)C[C@H]1C] | 1-((2R)-2-methyl-4-((2R,3R)- 2-methyl-1-(6-((3S,4R)-3- methyl-4-(4-methyl-1-(3- oxetanyl)-1H-pyrazol-5-yl)-1- piperidinyl)-2- (trifluoromethyl)-4- pyrimidinyl)-3-azetidinyl)-1- piperazinyl)-2-propen-1-one | E-6 | 603.1 |
| 2-472 | 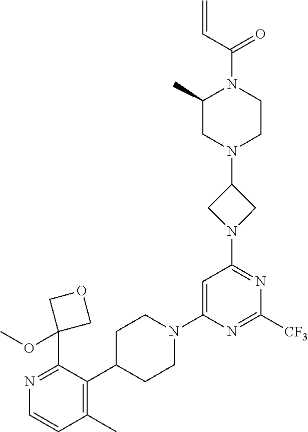 [SMILES: C=CC(=O)N1CCN(C2CN(c3cc(N4CCC(c5c(C)ccnc5C5(OC)COC5)CC4)nc(C(F)(F)F)n3)C2)C[C@H]1C] | 1-((2R)-4-(1-(6-(4-(2-(3- methoxy-3-oxetanyl)-4- methyl-3-pyridinyl)-1- piperidinyl)-2- (trifluoromethyl)-4- pyrimidinyl)-3-azetidinyl)-2- methyl-1-piperazinyl)-2- propen-1-one | E-4 | 616.0 |
| 2-482 | 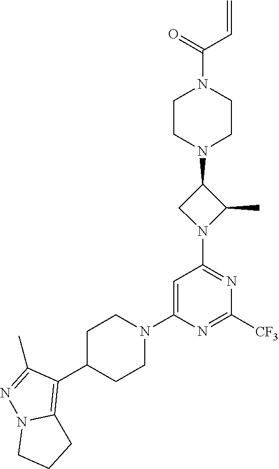 [SMILES: C=CC(=O)N1CCN([C@@H]2CN(c3cc(N4CCC(c5c(C)nn6c5CCC6)CC4)nc(C(F)(F)F)n3)[C@@H]2C)CC1] | 1-(4-((2R,3R)-2-methyl-1-(6- (4-(2-methyl-5,6-dihydro-4H- pyrrolo[1,2-b]pyrazol-3-yl)-1- piperidinyl)-2- (trifluoromethyl)-4- pyrimidinyl)-3-azetidinyl)-1- piperazinyl)-2-propen-1-one | E-8 | 559.3 |
| 2-483 | 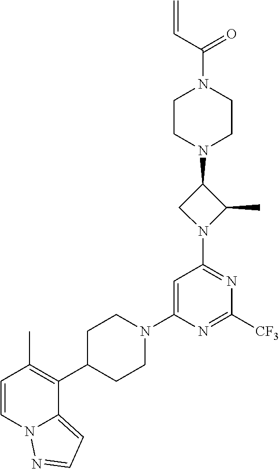 [SMILES: C=CC(=O)N1CCN([C@@H]2CN(c3cc(N4CCC(c5c(C)ccn6nccc56)CC4)nc(C(F)(F)F)n3)[C@@H]2C)CC1] | 1-(4-((2R,3R)-2-methyl-1-(6- (4-(5-methylpyrazolo[1,5- a]pyridin-4-yl)-1-piperidinyl)- 2-(trifluoromethyl)-4- pyrimidinyl)-3-azetidinyl)-1- piperazinyl)-2-propen-1-one | E-8 | 569.2 |
| 2-484 | 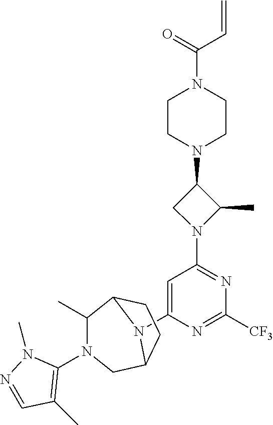 [SMILES: C=CC(=O)N1CCN([C@@H]2CN(c3cc(N4C5CCC4C(C)N(c4c(C)cnn4C)C5)nc(C(F)(F)F)n3)[C@@H]2C)CC1] | 1-(4-((2R,3R)-1-(6-(3-(1,4- dimethyl-1H-pyrazol-5-yl)-2- methyl-3,8- diazabicyclo[3.2.1]octan-8- yl)-2-(trifluoromethyl)-4- pyrimidinyl)-2-methyl-3- azetidinyl)-1-piperazinyl)-2- propen-1-one | E-1 | 574.3 |
| 2-487 | 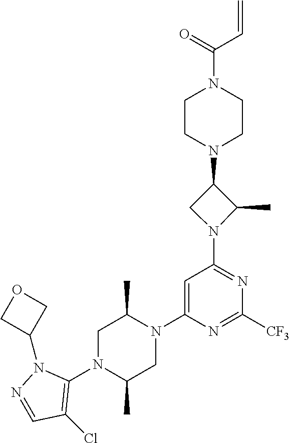 [SMILES: C=CC(=O)N1CCN([C@@H]2CN(c3cc(N4C[C@@H](C)N(c5c(Cl)cnn5C5COC5)C[C@H]4C)nc(C(F)(F)F)n3)[C@@H]2C)CC1] | 1-(4-((2R,3R)-1-(6-((2R,5R)- 4-(4-chloro-1-(3-oxetanyl)- 1H-pyrazol-5-yl)-2,5- dimethyl-1-piperazinyl)-2- (trifluoromethyl)-4- pyrimidinyl)-2-methyl-3- azetidinyl)-1-piperazinyl)-2- propen-1-one | E-8 | 624.3 |
| 2-489 | 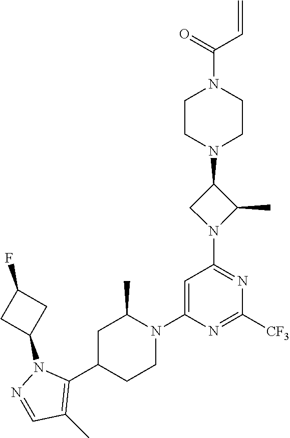 [SMILES: C=CC(=O)N1CCN([C@@H]2CN(c3cc(N4CCC(c5c(C)cnn5[C@H]5C[C@@H](F)C5)C[C@H]4C)nc(C(F)(F)F)n3)[C@@H]2C)CC1] | 1-(4-((2R,3R)-1-(6-((2R)-4- (1-((1s,3S)-3- fluorocyclobutyl)-4-methyl- 1H-pyrazol-5-yl)-2- methylpiperidin-1-yl)-2- (trifluoromethyl)pyrimidin-4- yl)-2-methylazetidin-3- yl)piperazin-1-yl)prop-2-en-1- one | E-8 | 605.1 |
| | mixture of isomers | | | |
| 2-490 | 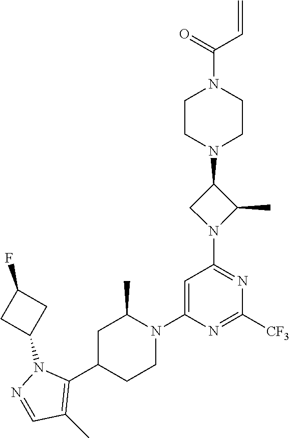 [SMILES: C=CC(=O)N1CCN([C@@H]2CN(c3cc(N4CCC(c5c(C)cnn5[C@H]5C[C@H](F)C5)C[C@H]4C)nc(C(F)(F)F)n3)[C@@H]2C)CC1] | 1-(4-((2R,3R)-1-(6-((2R)-4- (1-((1R,3R)-3- fluorocyclobutyl)-4-methyl- 1H-pyrazol-5-yl)-2- methylpiperidin-1-yl)-2- (trifluoromethyl)pyrimidin-4- yl)-2-methylazetidin-3- yl)piperazin-1-yl)prop-2-en-1- one | E-8 | 605.2 |
| | mixture of isomers | | | |
| 2-491 | 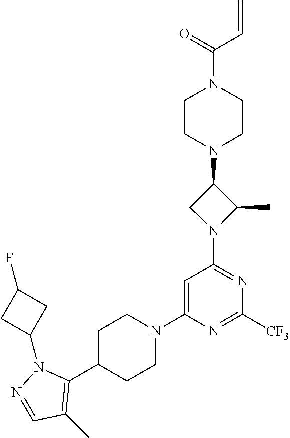 [SMILES: C=CC(=O)N1CCN([C@@H]2CN(c3cc(N4CCC(c5c(C)cnn5C5CC(F)C5)CC4)nc(C(F)(F)F)n3)[C@@H]2C)CC1] | 1-(4-((2R,3R)-1-(6-(4-(1-(3- fluorocyclobutyl)-4-methyl- 1H-pyrazol-5-yl)-1- piperidinyl)-2- (trifluoromethyl)-4- pyrimidinyl)-2-methyl-3- azetidinyl)-1-piperazinyl)-2- propen-1-one | E-8 | 591.1 |
| 2-492 | 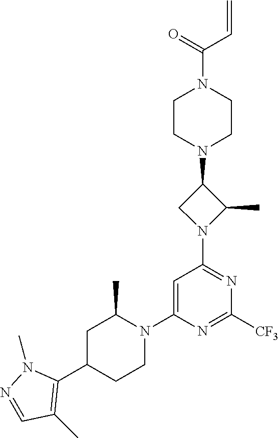 [SMILES: C=CC(=O)N1CCN([C@@H]2CN(c3cc(N4CCC(c5c(C)cnn5C)C[C@H]4C)nc(C(F)(F)F)n3)[C@@H]2C)CC1] | 1-(4-((2R,3R)-1-(6-((2R)-4- (1,4-dimethyl-1H-pyrazol-5- yl)-2-methylpiperidin-1-yl)-2- (trifluoromethyl)pyrimidin-4- yl)-2-methylazetidin-3- yl)piperazin-1-yl)prop-2-en-1- one | E-8 | 547.2 |
| 2-493 | 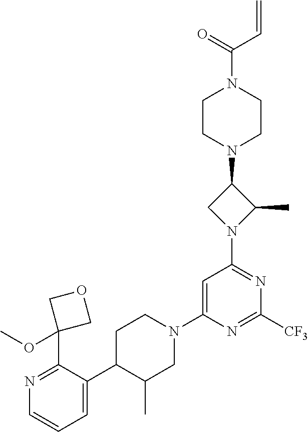 [SMILES: C=CC(=O)N1CCN([C@@H]2CN(c3cc(N4CCC(c5cccnc5C5(OC)COC5)C(C)C4)nc(C(F)(F)F)n3)[C@@H]2C)CC1] | 1-(4-((2R,3R)-1-(6-(4-(2-(3- methoxy-3-oxetanyl)-3- pyridinyl)-3-methyl-1- piperidinyl)-2- (trifluoromethyl)-4- pyrimidinyl)-2-methyl-3- azetidinyl)-1-piperazinyl)-2- propen-1-one | E-8 | 616.0 |
| 2-494 | 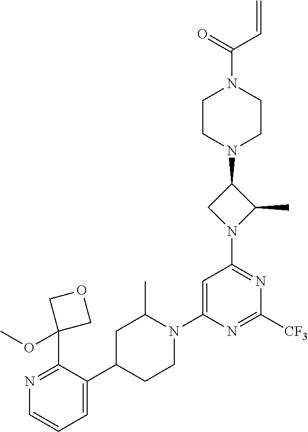 [SMILES: C=CC(=O)N1CCN([C@@H]2CN(c3cc(N4CCC(c5cccnc5C5(OC)COC5)CC4C)nc(C(F)(F)F)n3)[C@@H]2C)CC1] | 1-(4-((2R,3R)-1-(6-(4-(2-(3- methoxy-3-oxetanyl)-3- pyridinyl)-2-methyl-1- piperidinyl)-2- (trifluoromethyl)-4- pyrimidinyl)-2-methyl-3- azetidinyl)-1-piperazinyl)-2- propen-1-one | E-8 | 616.5 |
| 2-495 | 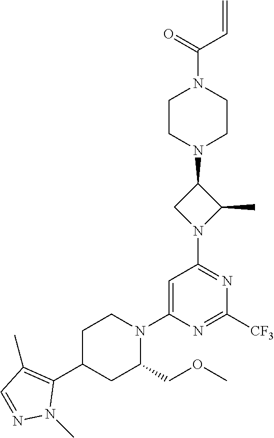 [SMILES: C=CC(=O)N1CCN([C@@H]2CN(c3cc(N4CCC(c5c(C)cnn5C)C[C@H]4COC)nc(C(F)(F)F)n3)[C@@H]2C)CC1] | 1-(4-((2R,3R)-1-(6-((2S)-4- (1,4-dimethyl-1H-pyrazol-5- yl)-2- (methoxymethyl)piperidin-1- yl)-2- (trifluoromethyl)pyrimidin-4- yl)-2-methylazetidin-3- yl)piperazin-1-yl)prop-2-en-1- one | E-8 | 577.2 |
| 2-496 | 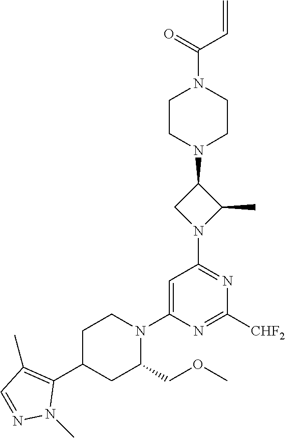 [SMILES: C=CC(=O)N1CCN([C@@H]2CN(c3cc(N4CCC(c5c(C)cnn5C)C[C@H]4COC)nc(C(F)F)n3)[C@@H]2C)CC1] | 1-(4-((2R,3R)-1-(2- (difluoromethyl)-6-((2S)-4- (1,4-dimethyl-1H-pyrazol-5- yl)-2- (methoxymethyl)piperidin-1- yl)pyrimidin-4-yl)-2- methylazetidin-3-yl)piperazin- 1-yl)prop-2-en-1-one | E-9 | 559.2 |
| 2-497 | 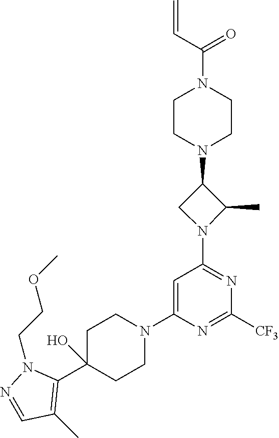 [SMILES: C=CC(=O)N1CCN([C@@H]2CN(c3cc(N4CCC(O)(c5c(C)cnn5CCOC)CC4)nc(C(F)(F)F)n3)[C@@H]2C)CC1] | 1-(4-((2R,3R)-1-(6-(4- hydroxy-4-(1-(2- methoxyethyl)-4-methyl-1H- pyrazol-5-yl)-1-piperidinyl)-2- (trifluoromethyl)-4- pyrimidinyl)-2-methyl-3- azetidinyl)-1-piperazinyl)-2- propen-1-one | E-8 | 594.0 |
| 2-498 | 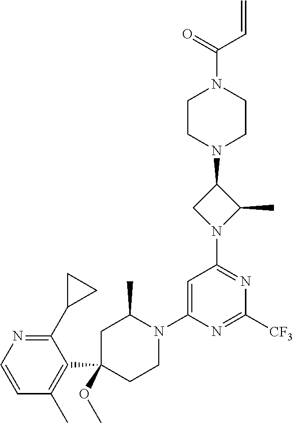 [SMILES: C=CC(=O)N1CCN([C@@H]2CN(c3cc(N4CC[C@](OC)(c5c(C)ccnc5C5CC5)C[C@H]4C)nc(C(F)(F)F)n3)[C@@H]2C)CC1] | 1-(4-((2R,3R)-1-(6-((2R,4R)- 4-(2-cyclopropyl-4-methyl-3- pyridinyl)-4-methoxy-2- methyl-1-piperidinyl)-2- (trifluoromethyl)-4- pyrimidinyl)-2-methyl-3- azetidinyl)-1-piperazinyl)-2- propen-1-one | E-8 | 603.0 |
| 2-499 | 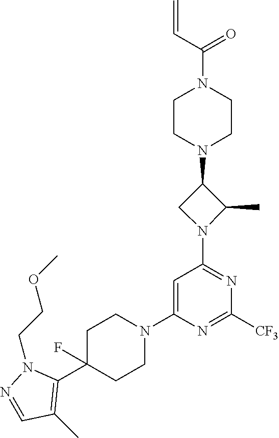 [SMILES: C=CC(=O)N1CCN([C@@H]2CN(c3cc(N4CCC(F)(c5c(C)cnn5CCOC)CC4)nc(C(F)(F)F)n3)[C@@H]2C)CC1] | 1-(4-((2R,3R)-1-(6-(4-fluoro- 4-(1-(2-methoxyethyl)-4- methyl-1H-pyrazol-5-yl)-1- piperidinyl)-2- (trifluoromethyl)-4- pyrimidinyl)-2-methyl-3- azetidinyl)-1-piperazinyl)-2- propen-1-one | E-8 | 595.0 |
| 2-501 | 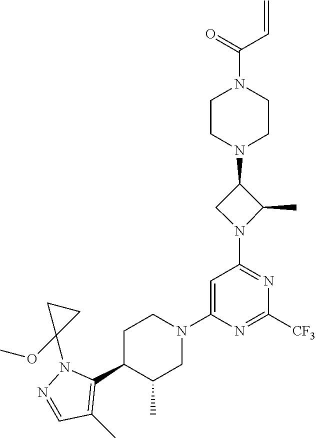 [SMILES: C=CC(=O)N1CCN([C@@H]2CN(c3cc(N4CC[C@H](c5c(C)cnn5C5(OC)CC5)[C@@H](C)C4)nc(C(F)(F)F)n3)[C@@H]2C)CC1] | 1-(4-((2R,3R)-1-(6-((3R,4S)- 4-(1-(1-methoxycyclopropyl)- 4-methyl-1H-pyrazol-5-yl)-3- methyl-1-piperidinyl)-2- (trifluoromethyl)-4- pyrimidinyl)-2-methyl-3- azetidinyl)-1-piperazinyl)-2- propen-1-one | E-8 | 603.4 |
| 2-502 | 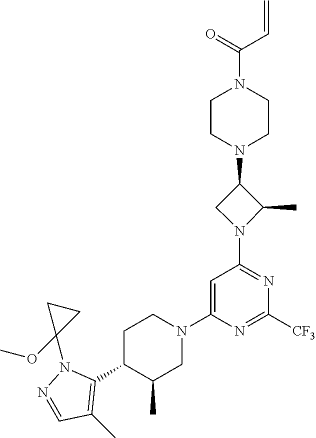 [SMILES: C=CC(=O)N1CCN([C@@H]2CN(c3cc(N4CC[C@@H](c5c(C)cnn5C5(OC)CC5)[C@H](C)C4)nc(C(F)(F)F)n3)[C@@H]2C)CC1] | 1-(4-((2R,3R)-1-(6-((3S,4R)- 4-(1-(1-methoxycyclopropyl)- 4-methyl-1H-pyrazol-5-yl)-3- methyl-1-piperidinyl)-2- (trifluoromethyl)-4- pyrimidinyl)-2-methyl-3- azetidinyl)-1-piperazinyl)-2- propen-1-one | E-8 | 603.4 |
| 2-503 | 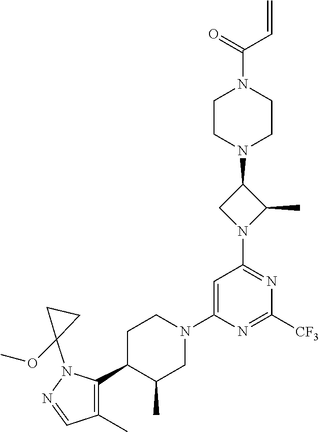 [SMILES: C=CC(=O)N1CCN([C@@H]2CN(c3cc(N4CC[C@H](c5c(C)cnn5C5(OC)CC5)[C@H](C)C4)nc(C(F)(F)F)n3)[C@@H]2C)CC1] | 1-(4-((2R,3R)-1-(6-((3S,4S)- 4-(1-(1-methoxycyclopropyl)- 4-methyl-1H-pyrazol-5-yl)-3- methyl-1-piperidinyl)-2- (trifluoromethyl)-4- pyrimidinyl)-2-methyl-3- azetidinyl)-1-piperazinyl)-2- propen-1-one | E-8 | 603.1 |
| 2-504 | 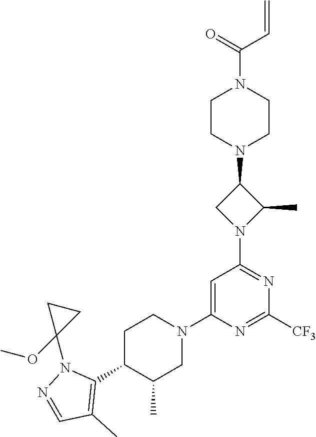 [SMILES: C=CC(=O)N1CCN([C@@H]2CN(c3cc(N4CC[C@@H](c5c(C)cnn5C5(OC)CC5)[C@@H](C)C4)nc(C(F)(F)F)n3)[C@@H]2C)CC1] | 1-(4-((2R,3R)-1-(6-((3R,4R)- 4-(1-(1-methoxycyclopropyl)- 4-methyl-1H-pyrazol-5-yl)-3- methyl-1-piperidinyl)-2- (trifluoromethyl)-4- pyrimidinyl)-2-methyl-3- azetidinyl)-1-piperazinyl)-2- propen-1-one | E-8 | 603.1 |
| 2-505 | 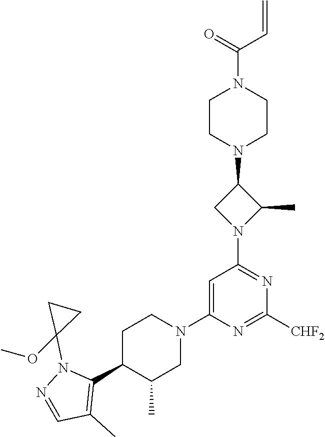 [SMILES: C=CC(=O)N1CCN([C@@H]2CN(c3cc(N4CC[C@H](c5c(C)cnn5C5(OC)CC5)[C@@H](C)C4)nc(C(F)F)n3)[C@@H]2C)CC1] | 1-(4-((2R,3R)-1-(2- (difluoromethyl)-6-((3R,4S)- 4-(1-(1-methoxycyclopropyl)- 4-methyl-1H-pyrazol-5-yl)-3- methyl-1-piperidinyl)-4- pyrimidinyl)-2-methyl-3- azetidinyl)-1-piperazinyl)-2- propen-1-one | E-9 | 585.4 |
| 2-506 | 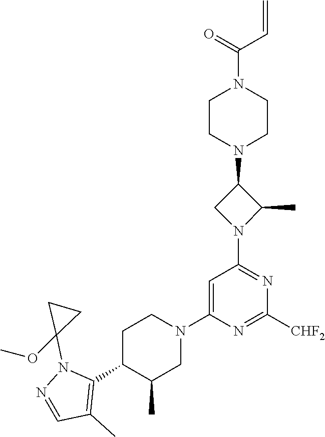 [SMILES: C=CC(=O)N1CCN([C@@H]2CN(c3cc(N4CC[C@@H](c5c(C)cnn5C5(OC)CC5)[C@H](C)C4)nc(C(F)F)n3)[C@@H]2C)CC1] | 1-(4-((2R,3R)-1-(2- (difluoromethyl)-6-((3S,4R)- 4-(1-(1-methoxycyclopropyl)- 4-methyl-1H-pyrazol-5-yl)-3- methyl-1-piperidinyl)-4- pyrimidinyl)-2-methyl-3- azetidinyl)-1-piperazinyl)-2- propen-1-one | E-9 | 585.4 |
| 2-507 | 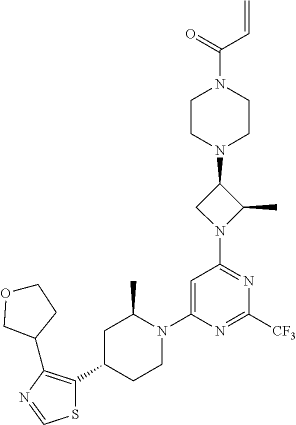 [SMILES: C=CC(=O)N1CCN([C@@H]2CN(c3cc(N4CC[C@H](c5scnc5C5CCOC5)C[C@H]4C)nc(C(F)(F)F)n3)[C@@H]2C)CC1] | 1-(4-((2R,3R)-2-methyl-1-(6- ((2R,4S)-2-methyl-4-(4- (tetrahydro-3-furanyl)-1,3- thiazol-5-yl)-1-piperidinyl)-2- (trifluoromethyl)-4- pyrimidinyl)-3-azetidinyl)-1- piperazinyl)-2-propen-1-one | E-8 | 606.2 |
| 2-508 | 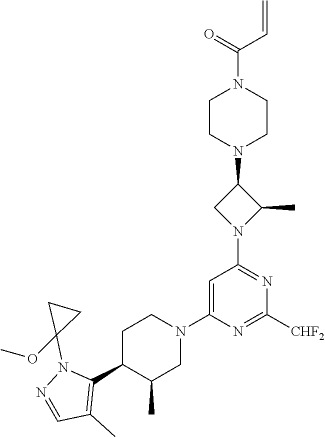 [SMILES: C=CC(=O)N1CCN([C@@H]2CN(c3cc(N4CC[C@H](c5c(C)cnn5C5(OC)CC5)[C@H](C)C4)nc(C(F)F)n3)[C@@H]2C)CC1] | 1-(4-((2R,3R)-1-(2- (difluoromethyl)-6-((3S,4S)-4- (1-(1-methoxycyclopropyl)-4- methyl-1H-pyrazol-5-yl)-3- methyl-1-piperidinyl)-4- pyrimidinyl)-2-methyl-3- azetidinyl)-1-piperazinyl)-2- propen-1-one | E-9 | 585.1 |
| 2-509 | 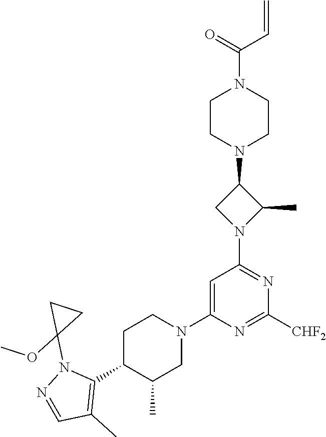 [SMILES: C=CC(=O)N1CCN([C@@H]2CN(c3cc(N4CC[C@@H](c5c(C)cnn5C5(OC)CC5)[C@@H](C)C4)nc(C(F)F)n3)[C@@H]2C)CC1] | 1-(4-((2R,3R)-1-(2- (difluoromethyl)-6-((3R,4R)- 4-(1-(1-methoxycyclopropyl)- 4-methyl-1H-pyrazol-5-yl)-3- methyl-1-piperidinyl)-4- pyrimidinyl)-2-methyl-3- azetidinyl)-1-piperazinyl)-2- propen-1-one | E-9 | 585.1 |
| 2-510 | 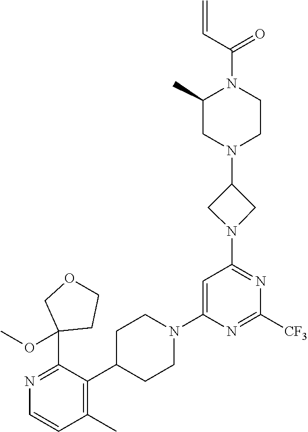 [SMILES: C=CC(=O)N1CCN(C2CN(c3cc(N4CCC(c5c(C)ccnc5C5(OC)CCOC5)CC4)nc(C(F)(F)F)n3)C2)C[C@H]1C] | 1-((2R)-4-(1-(6-(4-(2-(3- methoxytetrahydro-3-furanyl)- 4-methyl-3-pyridinyl)-1- piperidinyl)-2- (trifluoromethyl)-4- pyrimidinyl)-3-azetidinyl)-2- methyl-1-piperazinyl)-2- propen-1-one | E-4 | 630.3 |
| 2-511 | 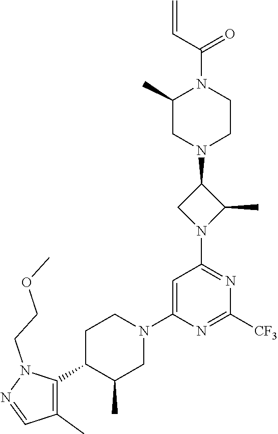 [SMILES: C=CC(=O)N1CCN([C@@H]2CN(c3cc(N4CC[C@@H](c5c(C)cnn5CCOC)[C@H](C)C4)nc(C(F)(F)F)n3)[C@@H]2C)C[C@H]1C] | 1-((2R)-4-((2R,3R)-1-(6- ((3S,4R)-4-(1-(2- methoxyethyl)-4-methyl-1H- pyrazol-5-yl)-3-methyl-1- piperidinyl)-2- (trifluoromethyl)-4- pyrimidinyl)-2-methyl-3- azetidinyl)-2-methyl-1- piperazinyl)-2-propen-1-one | E-6 | 605.1 |
| 2-512 | 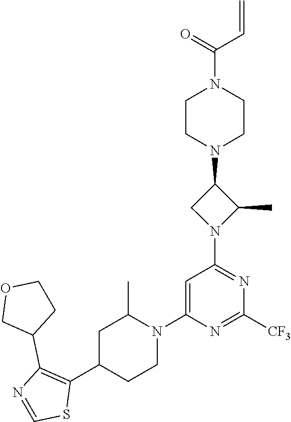 [SMILES: C=CC(=O)N1CCN([C@@H]2CN(c3cc(N4CCC(c5scnc5C5CCOC5)CC4C)nc(C(F)(F)F)n3)[C@@H]2C)CC1] | 1-(4-((2R,3R)-2-methyl-1-(6- (2-methyl-4-(4-(tetrahydro-3- furanyl)-1,3-thiazol-5-yl)-1- piperidinyl)-2- (trifluoromethyl)-4- pyrimidinyl)-3-azetidinyl)-1- piperazinyl)-2-propen-1-one | E-8 | 606.3 |
| | mixture of isomers | | | |
| 2-515 | 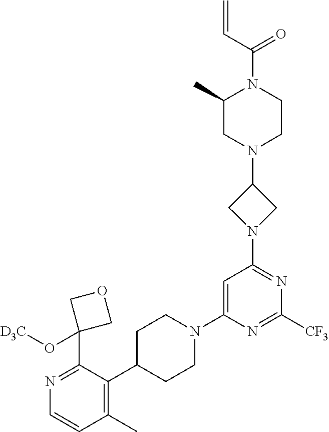 [SMILES: [2H]C([2H])([2H])OC1(c2nccc(C)c2C2CCN(c3cc(N4CC(N5CCN(C(=O)C=C)[C@H](C)C5)C4)nc(C(F)(F)F)n3)CC2)COC1] | (R)-1-(4-(1-(6-(4-(2-(3- (methoxy-d3)oxetan-3-yl)-4- methylpyridin-3-yl)piperidin- 1-yl)-2- (trifluoromethyl)pyrimidin-4- yl)azetidin-3-yl)-2- methylpiperazin-1-yl)prop-2- en-1-one | E-4 | 619.2 |
| 2-516 | 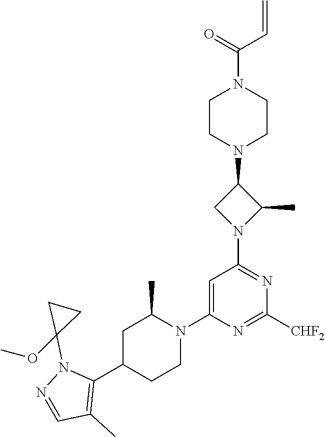 [SMILES: C=CC(=O)N1CCN([C@@H]2CN(c3cc(N4CCC(c5c(C)cnn5C5(OC)CC5)C[C@H]4C)nc(C(F)F)n3)[C@@H]2C)CC1] | 1-(4-((2R,3R)-1-(2- (difluoromethyl)-6-((2R)-4-(1- (1-methoxycyclopropyl)-4- methyl-1Hpyrazol-5-yl)-2- methylpiperidin-1- yl)pyrimidin-4-yl)-2- methylazetidin-3-yl)piperazin- 1-yl)prop-2-en-1-one | E-9 | 585.2 |
| 2-517 | 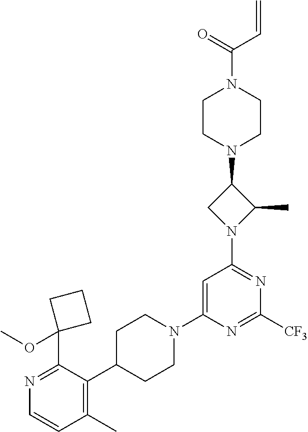 [SMILES: C=CC(=O)N1CCN([C@@H]2CN(c3cc(N4CCC(c5c(C)ccnc5C5(OC)CCC5)CC4)nc(C(F)(F)F)n3)[C@@H]2C)CC1] | 1-(4-((2R,3R)-1-(6-(4-(2-(1- methoxycyclobutyl)-4-methyl- 3-pyridinyl)-1-piperidinyl)-2- (trifluoromethyl)-4- pyrimidinyl)-2-methyl-3- azetidinyl)-1-piperazinyl)-2- propen-1-one | E-8 | 614.1 |
| 2-518 | 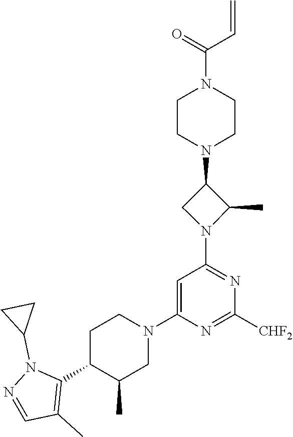 [SMILES: C=CC(=O)N1CCN([C@@H]2CN(c3cc(N4CC[C@@H](c5c(C)cnn5C5CC5)[C@H](C)C4)nc(C(F)F)n3)[C@@H]2C)CC1] | 1-(4-((2R,3R)-1-(6-((3S,4R)- 4-(1-cyclopropyl-4-methyl- 1H-pyrazol-5-yl)-3-methyl-1- piperidinyl)-2- (difluoromethyl)-4- pyrimidinyl)-2-methyl-3- azetidinyl)-1-piperazinyl)-2- propen-1-one | E-2 | 555.1 |
| 2-519 | 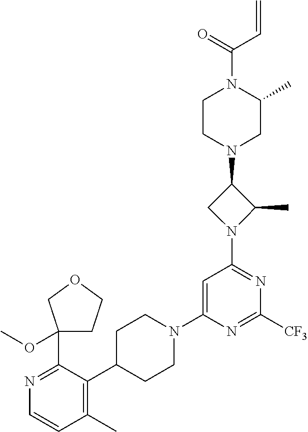 [SMILES: C=CC(=O)N1CCN([C@@H]2CN(c3cc(N4CCC(c5c(C)ccnc5C5(OC)CCOC5)CC4)nc(C(F)(F)F)n3)[C@@H]2C)C[C@H]1C] | 1-((2R)-4-((2R,3R)-1-(6-(4- (2-(3-methoxytetrahydro-3- furanyl)-4-methyl-3- pyridinyl)-1-piperidinyl)-2- (trifluoromethyl)-4- pyrimidinyl)-2-methyl-3- azetidinyl)-2-methyl-1- piperazinyl)-2-propen-1-one | E-6 | 649.9 |
| | mixture of isomers | | | |
| 2-520 | 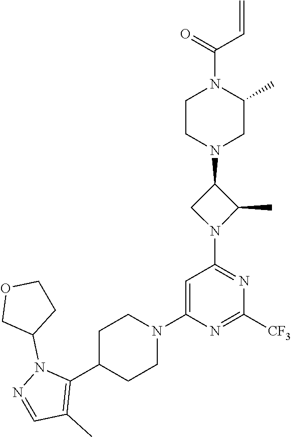 [SMILES: C=CC(=O)N1CCN([C@@H]2CN(c3cc(N4CCC(c5c(C)cnn5C5CCOC5)CC4)nc(C(F)(F)F)n3)[C@@H]2C)C[C@H]1C] | 1-((2R)-2-methyl-4-((2R,3R)- 2-methyl-1-(6-(4-(4-methyl-1- (tetrahydro-3-furanyl)-1H- pyrazol-5-yl)-1-piperidinyl)-2- (trifluoromethyl)-4- pyrimidinyl)-3-azetidinyl)-1- piperazinyl)-2-propen-1-one | E-6 | 603 |
| | mixture of isomers | | | |
| 2-521 | 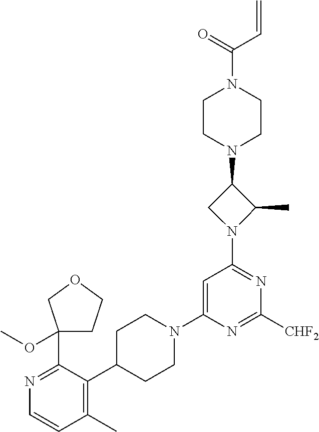 [SMILES: C=CC(=O)N1CCN([C@@H]2CN(c3cc(N4CCC(c5c(C)ccnc5C5(OC)CCOC5)CC4)nc(C(F)F)n3)[C@@H]2C)CC1] | 1-(4-((2R,3R)-1-(2- (difluoromethyl)-6-(4-(2-(3- methoxytetrahydro-3-furanyl)- 4-methyl-3-pyridinyl)-1- piperidinyl)-4-pyrimidinyl)-2- methyl-3-azetidinyl)-1- piperazinyl)-2-propen-1-one | E-2 | 603.1 |
| | mixture of isomers | | | |
| 2-522 | 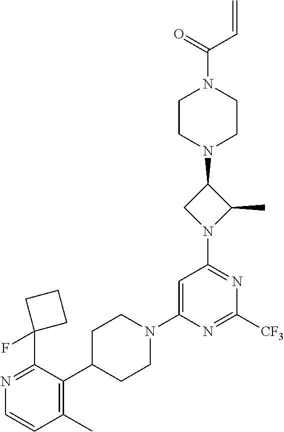 [SMILES: C=CC(=O)N1CCN([C@@H]2CN(c3cc(N4CCC(c5c(C)ccnc5C5(F)CCC5)CC4)nc(C(F)(F)F)n3)[C@@H]2C)CC1] | 1-(4-((2R,3R)-1-(6-(4-(2-(1- fluorocyclobutyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-8 | 602.1 |
| 2-523 | 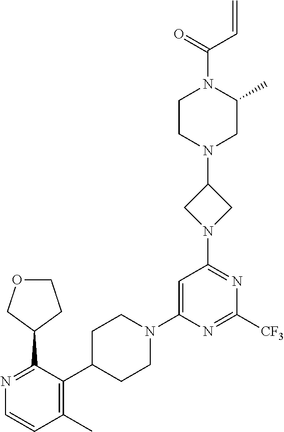 [SMILES: C=CC(=O)N1CCN(C2CN(c3cc(N4CCC(c5c(C)ccnc5[C@@H]5CCOC5)CC4)nc(C(F)(F)F)n3)C2)C[C@H]1C] | 1-((2R)-2-methyl-4-(1-(6-(4- (4-methyl-2-((3S)-tetrahydro- 3-furanyl)-3-pyridinyl)-1- piperidinyl)-2- (trifluoromethyl)-4- pyrimidinyl)-3-azetidinyl)-1- piperazinyl)-2-propen-1-one | E-4 | 600.1 |
| 2-525 | 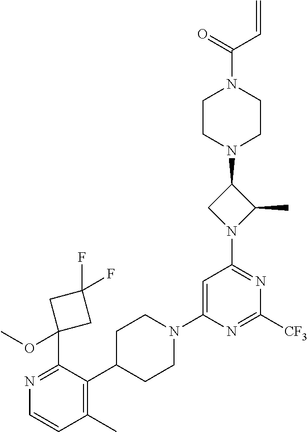 [SMILES: C=CC(=O)N1CCN([C@@H]2CN(c3cc(N4CCC(c5c(C)ccnc5C5(OC)CC(F)(F)C5)CC4)nc(C(F)(F)F)n3)[C@@H]2C)CC1] | 1-(4-((2R,3R)-1-(6-(4-(2-(3,3- difluoro-1- methoxycyclobutyl)-4-methyl- 3-pyridinyl)-1-piperidinyl)-2- (trifluoromethyl)-4- pyrimidinyl)-2-methyl-3- azetidinyl)-1-piperazinyl)-2- propen-1-one | E-8 | 649.9 |
| 2-527 | 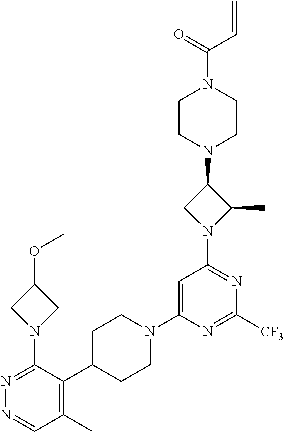 [SMILES: C=CC(=O)N1CCN([C@@H]2CN(c3cc(N4CCC(c5c(C)cnnc5N5CC(OC)C5)CC4)nc(C(F)(F)F)n3)[C@@H]2C)CC1] | 1-(4-((2R,3R)-1-(6-(4-(3-(3- methoxy-1-azetidinyl)-5- methyl-4-pyridazinyl)-1- piperidinyl)-2- (trifluoromethyl)-4- pyrimidinyl)-2-methyl-3- azetidinyl)-1-piperazinyl)-2- propen-1-one | E-8 | 616.0 |
| 2-528 | 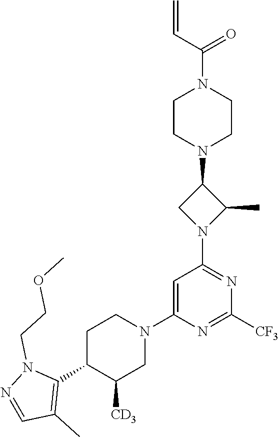 [SMILES: [2H]C([2H])([2H])[C@@H]1CN(c2cc(N3C[C@@H](N4CCN(C(=O)C=C)CC4)[C@H]3C)nc(C(F)(F)F)n2)CC[C@H]1c1c(C)cnn1CCOC] | 1-(4-((2R,3R)-1-(6-((3S,4R)- 4-(1-(2-methoxyethyl)-4- methyl-1H-pyrazol-5-yl)-3- (methyl-d3)piperidin-1-yl)-2- (trifluoromethyl)pyrimidin-4- yl)-2-methylazetidin-3- yl)piperazin-1-yl)prop-2-en-1- one | E-8 | 594.0 |
| 2-529 | 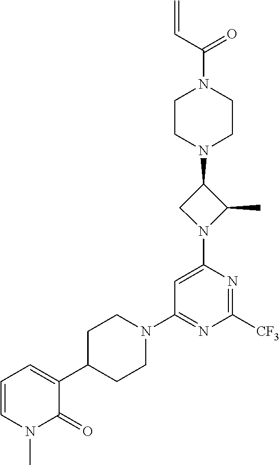 [SMILES: C=CC(=O)N1CCN([C@@H]2CN(c3cc(N4CCC(c5cccn(C)c5=O)CC4)nc(C(F)(F)F)n3)[C@@H]2C)CC1] | 1-methyl-3-(1-(6-((2R,3R)-2- methyl-3-(4-(2-propenoyl)-1- piperazinyl)-1-azetidinyl)-2- (trifluoromethyl)-4- pyrimidinyl)-4-piperidinyl)- 2(1H)-pyridinone | E-8 | 546.0 |
| 2-530 | 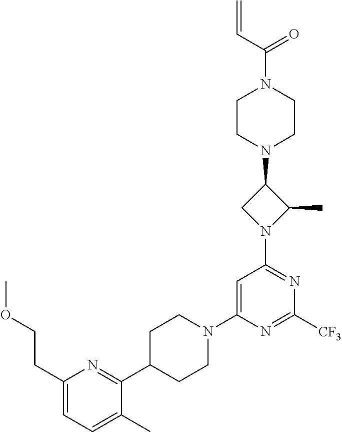 [SMILES: C=CC(=O)N1CCN([C@@H]2CN(c3cc(N4CCC(c5nc(CCOC)ccc5C)CC4)nc(C(F)(F)F)n3)[C@@H]2C)CC1] | 1-(4-((2R,3R)-1-(6-(4-(6-(2- methoxyethyl)-3-methyl-2- pyridinyl)-1-piperidinyl)-2- (trifluoromethyl)-4- pyrimidinyl)-2-methyl-3- azetidinyl)-1-piperazinyl)-2- propen-1-one | E-8 | 588.2 |
| 2-531 | 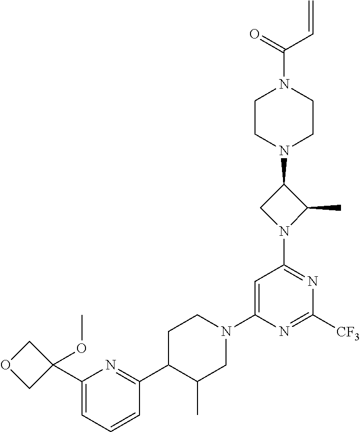 [SMILES: C=CC(=O)N1CCN([C@@H]2CN(c3cc(N4CCC(c5cccc(C6(OC)COC6)n5)C(C)C4)nc(C(F)(F)F)n3)[C@@H]2C)CC1] | 1-(4-((2R,3R)-1-(6-(4-(6-(3- methoxyoxetan-3-yl)pyridin- 2-yl)-3-methylpiperidin-1-yl)- 2-(trifluoromethyl)pyrimidin- 4-yl)-2-methylazetidin-3- yl)piperazin-1-yl)prop-2-en-1- one | E-8 | 616.2 |
| | mixture of trans isomers | | | |
| 2-532 | 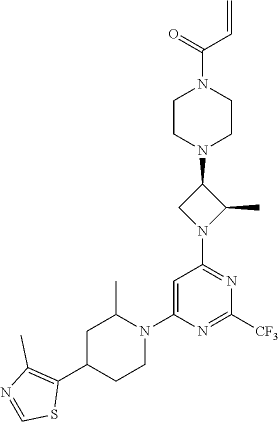 [SMILES: C=CC(=O)N1CCN([C@@H]2CN(c3cc(N4CCC(c5scnc5C)CC4C)nc(C(F)(F)F)n3)[C@@H]2C)CC1] | 1-(4-((2R,3R)-2-methyl-1-(6- (2-methyl-4-(4-methylthiazol- 5-yl)piperidin-1-yl)-2- (trifluoromethyl)pyrimidin-4- yl)azetidin-3-yl)piperazin-1- yl)prop-2-en-1-one | E-8 | 550.0 |
| 2-533 | 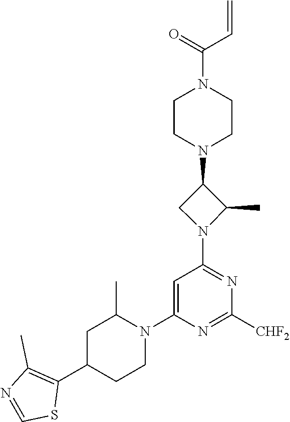 [SMILES: C=CC(=O)N1CCN([C@@H]2CN(c3cc(N4CCC(c5scnc5C)CC4C)nc(C(F)F)n3)[C@@H]2C)CC1] | 1-(4-((2R,3R)-1-(2- (difluoromethyl)-6-(2-methyl- 4-(4-methylthiazol-5- yl)piperidin-1-yl)pyrimidin-4- yl)-2-methylazetidin-3- yl)piperazin-1-yl)prop-2-en-1- one | E-9 | 532.0 |
| 2-535 | 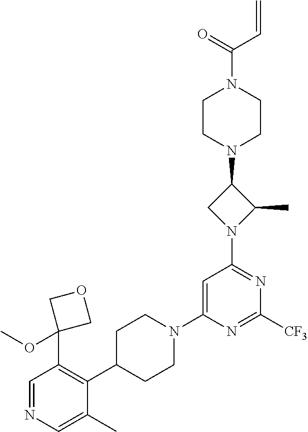 [SMILES: C=CC(=O)N1CCN([C@@H]2CN(c3cc(N4CCC(c5c(C)cncc5C5(OC)COC5)CC4)nc(C(F)(F)F)n3)[C@@H]2C)CC1] | 1-(4-((2R,3R)-1-(6-(4-(3-(3- methoxy-3-oxetanyl)-5- methyl-4-pyridinyl)-1- piperidinyl)-2- (trifluoromethyl)-4- pyrimidinyl)-2-methyl-3- azetidinyl)-1-piperazinyl)-2- propen-1-one | E-1 | 616.3 |
| 2-536 | 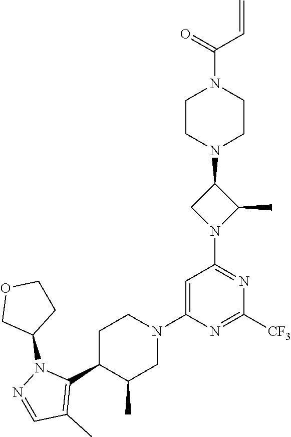 [SMILES: C=CC(=O)N1CCN([C@@H]2CN(c3cc(N4CC[C@H](c5c(C)cnn5[C@@H]5CCOC5)[C@H](C)C4)nc(C(F)(F)F)n3)[C@@H]2C)CC1] | 1-(4-((2R,3R)-2-methyl-1-(6- ((3S,4S)-3-methyl-4-(4- methyl-1-((3R)-tetrahydro-3- furanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 603.4 |
| 2-537 | 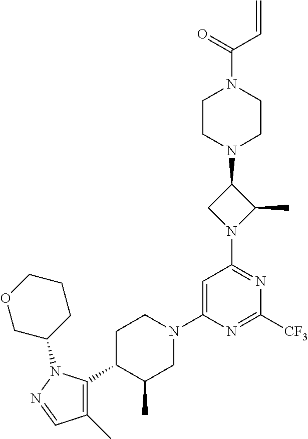 [SMILES: C=CC(=O)N1CCN([C@@H]2CN(c3cc(N4CC[C@@H](c5c(C)cnn5[C@H]5CCCOC5)[C@H](C)C4)nc(C(F)(F)F)n3)[C@@H]2C)CC1] | 1-(4-((2R,3R)-2-methyl-1-(6- ((3S,4R)-3-methyl-4-(4- methyl-1-((3S)-tetrahydro-2H- pyran-3-yl)-1H-pyrazol-5-yl)- 1-piperidinyl)-2- (trifluoromethyl)-4- pyrimidinyl)-3-azetidinyl)-1- piperazinyl)-2-propen-1-one | E-1 | 617.3 |
| 2-538 | 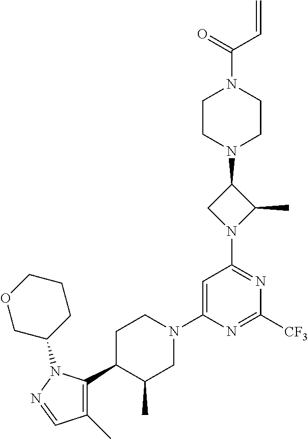 [SMILES: C=CC(=O)N1CCN([C@@H]2CN(c3cc(N4CC[C@H](c5c(C)cnn5[C@H]5CCCOC5)[C@H](C)C4)nc(C(F)(F)F)n3)[C@@H]2C)CC1] | 1-(4-((2R,3R)-2-methyl-1-(6- ((3S,4S)-3-methyl-4-(4- methyl-1-((3S)-tetrahydro-2H- pyran-3-yl)-1H-pyrazol-5-yl)- 1-piperidinyl)-2- (trifluoromethyl)-4- pyrimidinyl)-3-azetidinyl)-1- piperazinyl)-2-propen-1-one | E-1 | 617.3 |
| 2-539 | 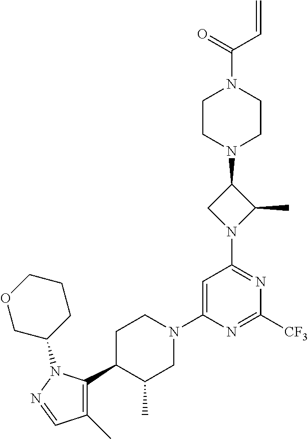 [SMILES: C=CC(=O)N1CCN([C@@H]2CN(c3cc(N4CC[C@H](c5c(C)cnn5[C@H]5CCCOC5)[C@@H](C)C4)nc(C(F)(F)F)n3)[C@@H]2C)CC1] | 1-(4-((2R,3R)-2-methyl-1-(6- ((3R,4S)-3-methyl-4-(4- methyl-1-((3S)-tetrahydro-2H- pyran-3-yl)-1H-pyrazol-5-yl)- 1-piperidinyl)-2- (trifluoromethyl)-4- pyrimidinyl)-3-azetidinyl)-1- piperazinyl)-2-propen-1-one | E-1 | 617.4 |
| 2-540 | 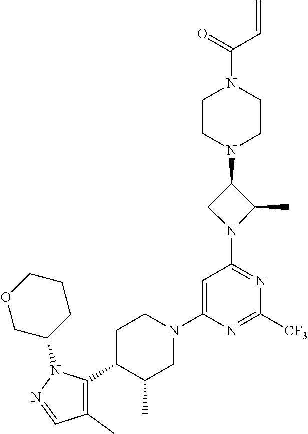 [SMILES: C=CC(=O)N1CCN([C@@H]2CN(c3cc(N4CC[C@@H](c5c(C)cnn5[C@H]5CCCOC5)[C@@H](C)C4)nc(C(F)(F)F)n3)[C@@H]2C)CC1] | 1-(4-((2R,3R)-2-methyl-1-(6- ((3R,4R)-3-methyl-4-(4- methyl-1-((3S)-tetrahydro-2H- pyran-3-yl)-1H-pyrazol-5-yl)- 1-piperidinyl)-2- (trifluoromethyl)-4- pyrimidinyl)-3-azetidinyl)-1- piperazinyl)-2-propen-1-one | E-1 | 617.3 |
| 2-541 | 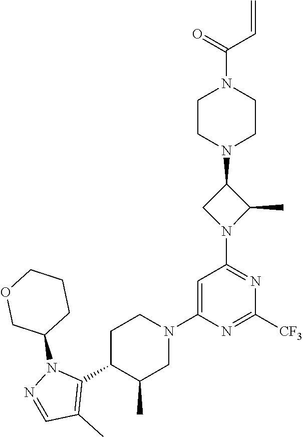 [SMILES: C=CC(=O)N1CCN([C@@H]2CN(c3cc(N4CC[C@@H](c5c(C)cnn5[C@@H]5CCCOC5)[C@H](C)C4)nc(C(F)(F)F)n3)[C@@H]2C)CC1] | 1-(4-((2R,3R)-2-methyl-1-(6- ((3S,4R)-3-methyl-4-(4- methyl-1-((3R)-tetrahydro- 2H-pyran-3-yl)-1H-pyrazol-5- yl)-1-piperidinyl)-2- (trifluoromethyl)-4- pyrimidinyl)-3-azetidinyl)-1- piperazinyl)-2-propen-1-one | E-1 | 617.4 |
| 2-542 | 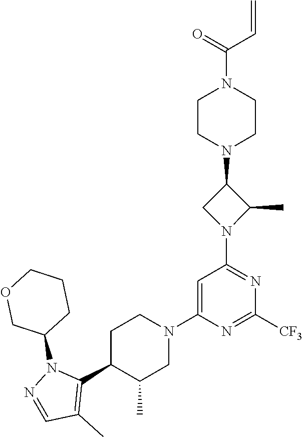 [SMILES: C=CC(=O)N1CCN([C@@H]2CN(c3cc(N4CC[C@H](c5c(C)cnn5[C@@H]5CCCOC5)[C@@H](C)C4)nc(C(F)(F)F)n3)[C@@H]2C)CC1] | 1-(4-((2R,3R)-2-methyl-1-(6- ((3R,4S)-3-methyl-4-(4- methyl-1-((3R)-tetrahydro- 2H-pyran-3-yl)-1H-pyrazol-5- yl)-1-piperidinyl)-2- (trifluoromethyl)-4- pyrimidinyl)-3-azetidinyl)-1- piperazinyl)-2-propen-1-one | E-1 | 617.3 |
| 2-543 | 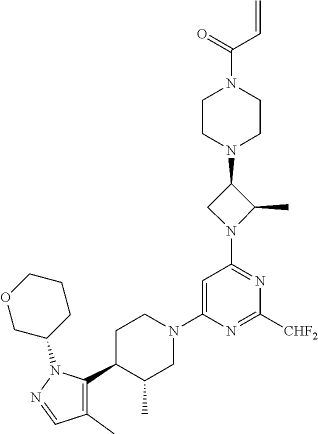 [SMILES: C=CC(=O)N1CCN([C@@H]2CN(c3cc(N4CC[C@H](c5c(C)cnn5[C@H]5CCCOC5)[C@@H](C)C4)nc(C(F)F)n3)[C@@H]2C)CC1] | 1-(4-((2R,3R)-1-(2- (difluoromethyl)-6-((3R,4S)- 3-methyl-4-(4-methyl-1-((3S)- tetrahydro-2H-pyran-3-yl)- 1H-pyrazol-5-yl)-1- piperidinyl)-4-pyrimidinyl)-2- methyl-3-azetidinyl)-1- piperazinyl)-2-propen-1-one | E-9 | 599.4 |
| 2-544 | 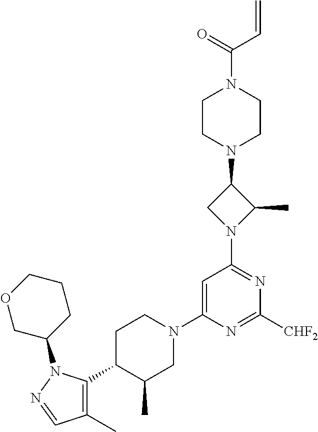 [SMILES: C=CC(=O)N1CCN([C@@H]2CN(c3cc(N4CC[C@@H](c5c(C)cnn5[C@@H]5CCCOC5)[C@H](C)C4)nc(C(F)F)n3)[C@@H]2C)CC1] | 1-(4-((2R,3R)-1-(2- (difluoromethyl)-6-((3S,4R)- 3-methyl-4-(4-methyl-1- ((3R)-tetrahydro-2H-pyran-3- yl)-1H-pyrazol-5-yl)-1- piperidinyl)-4-pyrimidinyl)-2- methyl-3-azetidinyl)-1- piperazinyl)-2-propen-1-one | E-9 | 599.4 |
| 2-545 | 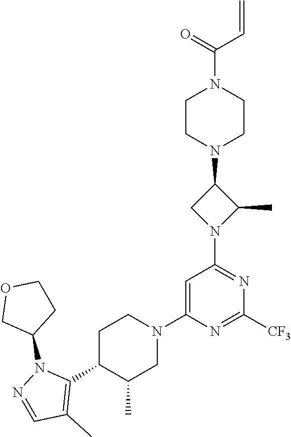 [SMILES: C=CC(=O)N1CCN([C@@H]2CN(c3cc(N4CC[C@@H](c5c(C)cnn5[C@@H]5CCOC5)[C@@H](C)C4)nc(C(F)(F)F)n3)[C@@H]2C)CC1] | 1-(4-((2R,3R)-2-methyl-1-(6- ((3R,4R)-3-methyl-4-(4- methyl-1-((3R)-tetrahydro-3- furanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 603.3 |
| 2-546 | 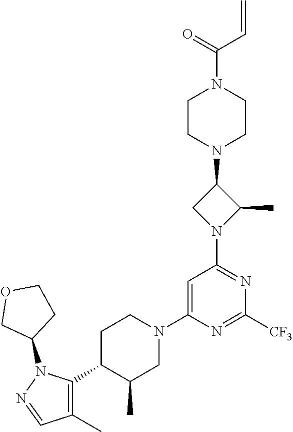 [SMILES: C=CC(=O)N1CCN([C@@H]2CN(c3cc(N4CC[C@@H](c5c(C)cnn5[C@@H]5CCOC5)[C@H](C)C4)nc(C(F)(F)F)n3)[C@@H]2C)CC1] | 1-(4-((2R,3R)-2-methyl-1-(6- ((3S,4R)-3-methyl-4-(4- methyl-1-((3R)-tetrahydro-3- furanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 603.3 |
| 2-547 | 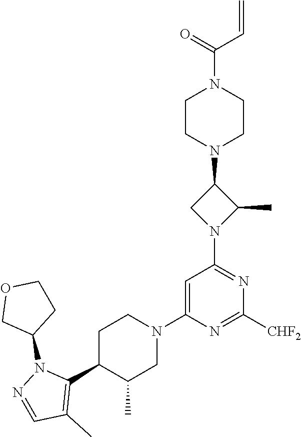 [SMILES: C=CC(=O)N1CCN([C@@H]2CN(c3cc(N4CC[C@H](c5c(C)cnn5[C@@H]5CCOC5)[C@@H](C)C4)nc(C(F)F)n3)[C@@H]2C)CC1] | 1-(4-((2R,3R)-1-(2- (difluoromethyl)-6-((3R,4S)- 3-methyl-4-(4-methyl-1- ((3R)-tetrahydro-3-furanyl)- 1H-pyrazol-5-yl)-1- piperidinyl)-4-pyrimidinyl)-2- methyl-3-azetidinyl)-1- piperazinyl)-2-propen-1-one | E-9 | 585.3 |
| 2-548 | 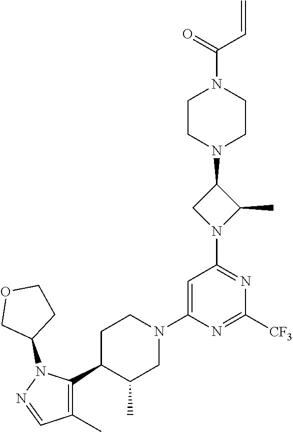 [SMILES: C=CC(=O)N1CCN([C@@H]2CN(c3cc(N4CC[C@H](c5c(C)cnn5[C@@H]5CCOC5)[C@@H](C)C4)nc(C(F)(F)F)n3)[C@@H]2C)CC1] | 1-(4-((2R,3R)-2-methyl-1-(6- ((3R,4S)-3-methyl-4-(4- methyl-1-((3R)-tetrahydro-3- furanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 603.3 |
| 2-549 | 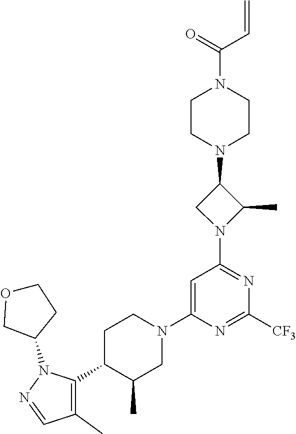 [SMILES: C=CC(=O)N1CCN([C@@H]2CN(c3cc(N4CC[C@@H](c5c(C)cnn5[C@H]5CCOC5)[C@H](C)C4)nc(C(F)(F)F)n3)[C@@H]2C)CC1] | 1-(4-((2R,3R)-2-methyl-1-(6- ((3S,4R)-3-methyl-4-(4- methyl-1-((3S)-tetrahydro-3- furanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 619.2 |
| 2-550 | 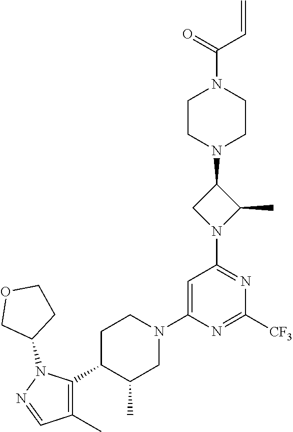 [SMILES: C=CC(=O)N1CCN([C@@H]2CN(c3cc(N4CC[C@@H](c5c(C)cnn5[C@H]5CCOC5)[C@@H](C)C4)nc(C(F)(F)F)n3)[C@@H]2C)CC1] | 1-(4-((2R,3R)-2-methyl-1-(6- ((3R,4R)-3-methyl-4-(4- methyl-1-((3S)-tetrahydro-3- furanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 603.4 |
| 2-551 | 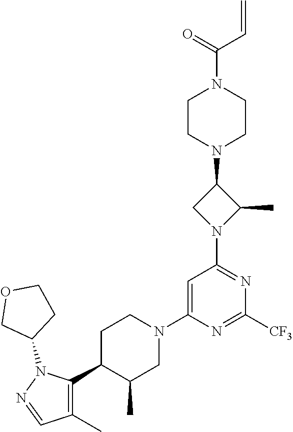 [SMILES: C=CC(=O)N1CCN([C@@H]2CN(c3cc(N4CC[C@H](c5c(C)cnn5[C@H]5CCOC5)[C@H](C)C4)nc(C(F)(F)F)n3)[C@@H]2C)CC1] | 1-(4-((2R,3R)-2-methyl-1-(6- ((3S,4S)-3-methyl-4-(4- methyl-1-((3S)-tetrahydro-3- furanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 603.2 |
| 2-552 | 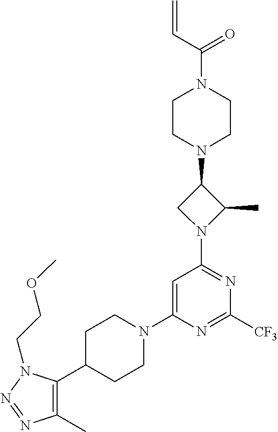 [SMILES: C=CC(=O)N1CCN([C@@H]2CN(c3cc(N4CCC(c5c(C)nnn5CCOC)CC4)nc(C(F)(F)F)n3)[C@@H]2C)CC1] | 1-(4-((2R,3R)-1-(6-(4-(1-(2- methoxyethyl)-4-methyl-1H- 1,2,3-triazol-5-yl)-1- piperidinyl)-2- (trifluoromethyl)-4- pyrimidinyl)-2-methyl-3- azetidinyl)-1-piperazinyl)-2- propen-1-one | E-1 | 578.2 |
| 2-553 | 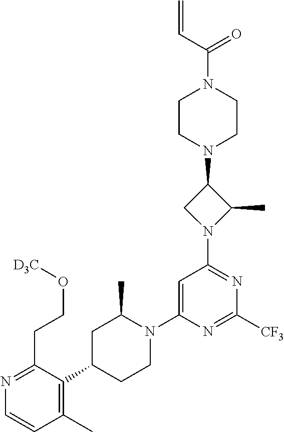 [SMILES: [2H]C([2H])([2H])OCCc1nccc(C)c1[C@H]1CCN(c2cc(N3C[C@@H](N4CCN(C(=O)C=C)CC4)[C@H]3C)nc(C(F)(F)F)n2)[C@H](C)C1] | 1-(4-((2R,3R)-1-(6-((2R,4S)- 4-(2-(2-(methoxy-d3)ethyl)-4- methylpyridin-3-yl)-2- methylpiperidin-1-yl)-2- (trifluoromethyl)pyrimidin-4- yl)-2-methylazetidin-3- yl)piperazin-1-yl)prop-2-en-1- one | E-1 | 605.4 |
| 2-554 | 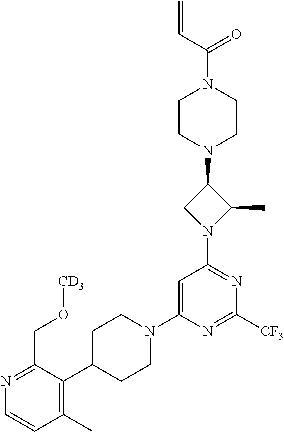 [SMILES: [2H]C([2H])([2H])OCc1nccc(C)c1C1CCN(c2cc(N3C[C@@H](N4CCN(C(=O)C=C)CC4)[C@H]3C)nc(C(F)(F)F)n2)CC1] | 1-(4-((2R,3R)-1-(6-(4-(2- ((methoxy-d3)methyl)-4- methylpyridin-3-yl)piperidin- 1-yl)-2- (trifluoromethyl)pyrimidin-4- yl)-2-methylazetidin-3- yl)piperazin-1-yl)prop-2-en-1- one | E-1 | 587.2 |
| 2-555 | 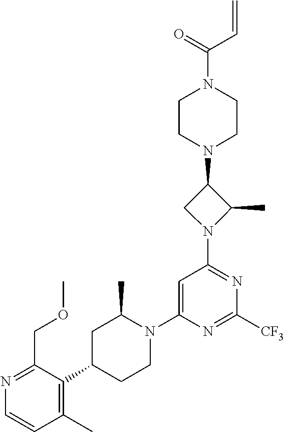 [SMILES: C=CC(=O)N1CCN([C@@H]2CN(c3cc(N4CC[C@H](c5c(C)ccnc5COC)C[C@H]4C)nc(C(F)(F)F)n3)[C@@H]2C)CC1] | 1-(4-((2R,3R)-1-(6-((2R,4S)- 4-(2-(methoxymethyl)-4- methyl-3-pyridinyl)-2-methyl- 1-piperidinyl)-2- (trifluoromethyl)-4- pyrimidinyl)-2-methyl-3- azetidinyl)-1-piperazinyl)-2- propen-1-one | E-8 | 588.4 |
| 2-556 | 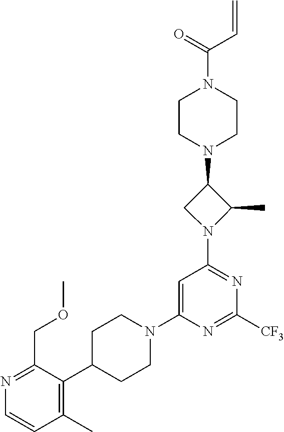 [SMILES: C=CC(=O)N1CCN([C@@H]2CN(c3cc(N4CCC(c5c(C)ccnc5COC)CC4)nc(C(F)(F)F)n3)[C@@H]2C)CC1] | 1-(4-((2R,3R)-1-(6-(4-(2- (methoxymethyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-1 | 574.3 |
| 2-559 | 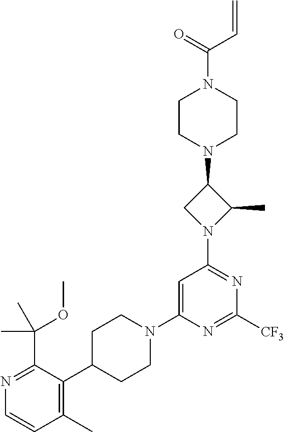 [SMILES: C=CC(=O)N1CCN([C@@H]2CN(c3cc(N4CCC(c5c(C)ccnc5C(C)(C)OC)CC4)nc(C(F)(F)F)n3)[C@@H]2C)CC1] | 1-(4-((2R,3R)-1-(6-(4-(2-(2- methoxy-2-propanyl)-4- methyl-3-pyridinyl)-1- piperidinyl)-2- (trifluoromethyl)-4- pyrimidinyl)-2-methyl-3- azetidinyl)-1-piperazinyl)-2- propen-1-one | E-1 | 602.3 |
| 2-560 | 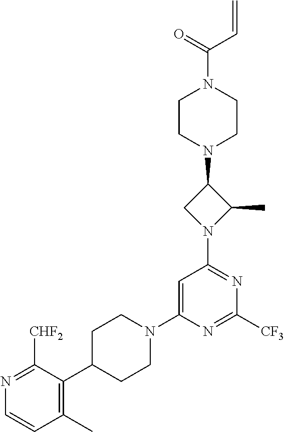 [SMILES: C=CC(=O)N1CCN([C@@H]2CN(c3cc(N4CCC(c5c(C)ccnc5C(F)F)CC4)nc(C(F)(F)F)n3)[C@@H]2C)CC1] | 1-(4-((2R,3R)-1-(6-(4-(2- (difluoromethyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-1 | 580.3 |
| 2-561 | 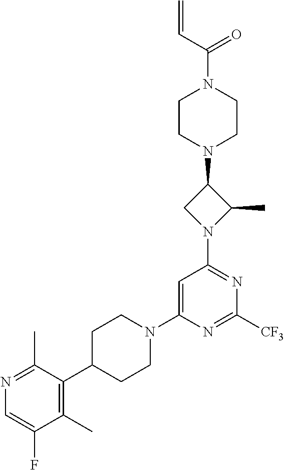 [SMILES: C=CC(=O)N1CCN([C@@H]2CN(c3cc(N4CCC(c5c(C)ncc(F)c5C)CC4)nc(C(F)(F)F)n3)[C@@H]2C)CC1] | 1-(4-((2R,3R)-1-(6-(4-(5- fluoro-2,4-dimethyl-3- pyridinyl)-1-piperidinyl)-2- (trifluoromethyl)-4- pyrimidinyl)-2-methyl-3- azetidinyl)-1-piperazinyl)-2- propen-1-one | E-8 | 562.3 |
| 2-562 | 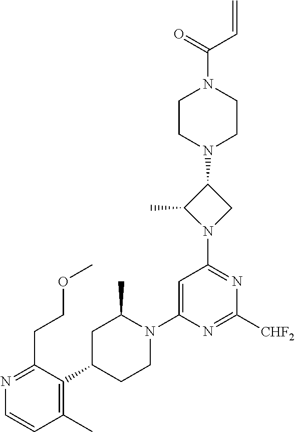 [SMILES: C=CC(=O)N1CCN([C@@H]2CN(c3cc(N4CC[C@H](c5c(C)ccnc5CCOC)C[C@H]4C)nc(C(F)F)n3)[C@@H]2C)CC1] | 1-(4-((2R,3R)-1-(2- (difluoromethyl)-6-((2R,4S)- 4-(2-(2-methoxyethyl)-4- methyl-3-pyridinyl)-2-methyl- 1-piperidinyl)-4-pyrimidinyl)- 2-methyl-3-azetidinyl)-1- piperazinyl)-2-propen-1-one | E-2 | 584.4 |
| 2-563 | 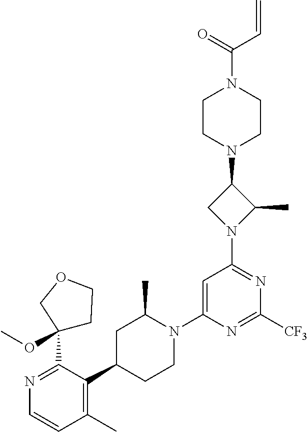 [SMILES: C=CC(=O)N1CCN([C@@H]2CN(c3cc(N4CC[C@@H](c5c(C)ccnc5[C@@]5(OC)CCOC5)C[C@H]4C)nc(C(F)(F)F)n3)[C@@H]2C)CC1] | 1-(4-((2R,3R)-1-(6-((2R,4R)- 4-(2-((3S)-3- methoxytetrahydro-3-furanyl)- 4-methyl-3-pyridinyl)-2- methyl-1-piperidinyl)-2- (trifluoromethyl)-4- pyrimidinyl)-2-methyl-3- azetidinyl)-1-piperazinyl)-2- propen-1-one | E-8 | 644.4 |
| 2-564 | 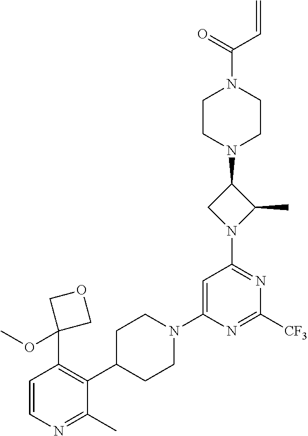 [SMILES: C=CC(=O)N1CCN([C@@H]2CN(c3cc(N4CCC(c5c(C6(OC)COC6)ccnc5C)CC4)nc(C(F)(F)F)n3)[C@@H]2C)CC1] | 1-(4-((2R,3R)-1-(6-(4-(4-(3- methoxy-3-oxetanyl)-2- methyl-3-pyridinyl)-1- piperidinyl)-2- (trifluoromethyl)-4- pyrimidinyl)-2-methyl-3- azetidinyl)-1-piperazinyl)-2- propen-1-one | E-1 | 616.3 |
| 2-565 | 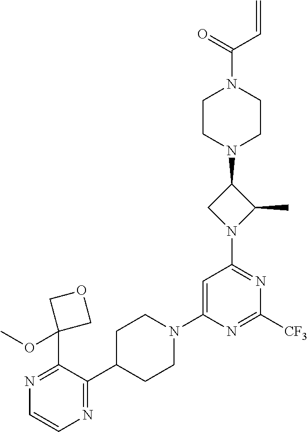 [SMILES: C=CC(=O)N1CCN([C@@H]2CN(c3cc(N4CCC(c5nccnc5C5(OC)COC5)CC4)nc(C(F)(F)F)n3)[C@@H]2C)CC1] | 1-(4-((2R,3R)-1-(6-(4-(3-(3- methoxy-3-oxetanyl)-2- pyrazinyl)-1-piperidinyl)-2- (trifluoromethyl)-4- pyrimidinyl)-2-methyl-3- azetidinyl)-1-piperazinyl)-2- propen-1-one | E-1 | 603.3 |
| 2-566 | 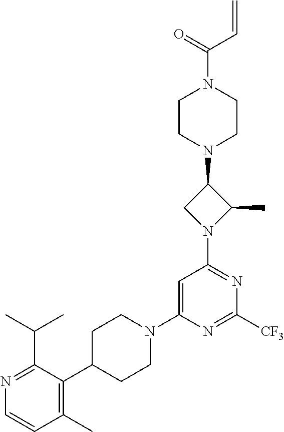 [SMILES: C=CC(=O)N1CCN([C@@H]2CN(c3cc(N4CCC(c5c(C)ccnc5C(C)C)CC4)nc(C(F)(F)F)n3)[C@@H]2C)CC1] | 1-(4-((2R,3R)-2-methyl-1-(6- (4-(4-methyl-2-(2-propanyl)- 3-pyridinyl)-1-piperidinyl)-2- (trifluoromethyl)-4- pyrimidinyl)-3-azetidinyl)-1- piperazinyl)-2-propen-1-one | E-1 | 572.4 |
| 2-567 | 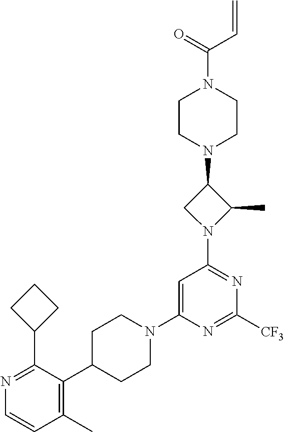 [SMILES: C=CC(=O)N1CCN([C@@H]2CN(c3cc(N4CCC(c5c(C)ccnc5C5CCC5)CC4)nc(C(F)(F)F)n3)[C@@H]2C)CC1] | 1-(4-((2R,3R)-1-(6-(4-(2- cyclobutyl-4-methyl-3- pyridinyl)-1-piperidinyl)-2- (trifluoromethyl)-4- pyrimidinyl)-2-methyl-3- azetidinyl)-1-piperazinyl)-2- propen-1-one | E-1 | 584.3 |
| 2-568 | 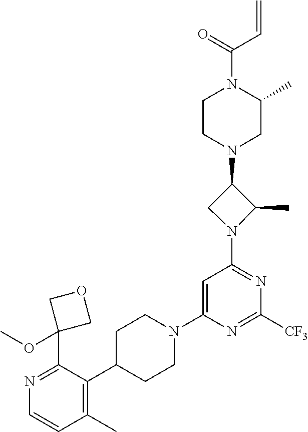 [SMILES: C=CC(=O)N1CCN([C@@H]2CN(c3cc(N4CCC(c5c(C)ccnc5C5(OC)COC5)CC4)nc(C(F)(F)F)n3)[C@@H]2C)C[C@H]1C] | 1-((2R)-4-((2R,3R)-1-(6-(4- (2-(3-methoxy-3-oxetanyl)-4- methyl-3-pyridinyl)-1- piperidinyl)-2- (trifluoromethyl)-4- pyrimidinyl)-2-methyl-3- azetidinyl)-2-methyl-1- piperazinyl)-2-propen-1-one | E-6 | 630.4 |
| 2-569 | 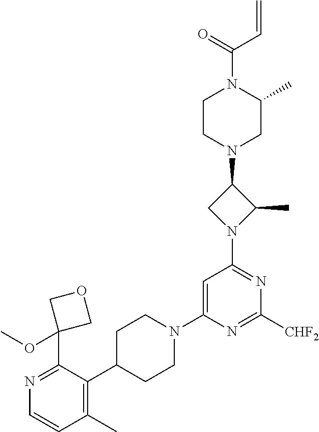 [SMILES: C=CC(=O)N1CCN([C@@H]2CN(c3cc(N4CCC(c5c(C)ccnc5C5(OC)COC5)CC4)nc(C(F)F)n3)[C@@H]2C)C[C@H]1C] | 1-((2R)-4-((2R,3R)-1-(2- (difluoromethyl)-6-(4-(2-(3- methoxy-3-oxetanyl)-4- methyl-3-pyridinyl)-1- piperidinyl)-4-pyrimidinyl)-2- methyl-3-azetidinyl)-2- methyl-1-piperazinyl)-2- propen-1-one | E4-1 | 612.4 |
| 2-570 | 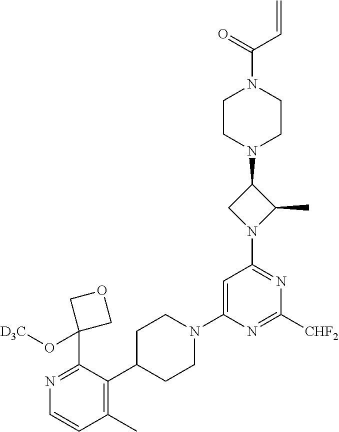 [SMILES: [2H]C([2H])([2H])OC1(c2nccc(C)c2C2CCN(c3cc(N4C[C@@H](N5CCN(C(=O)C=C)CC5)[C@H]4C)nc(C(F)F)n3)CC2)COC1] | 1-(4-((2R,3R)-1-(2- (difluoromethyl)-6-(4-(2-(3- (methoxy-d3)oxetan-3-yl)-4- methylpyridin-3-yl)piperidin- 1-yl)pyrimidin-4-yl)-2- methylazetidin-3-yl)piperazin- 1-yl)prop-2-en-1-one | E-2 | 601.3 |
| 2-583 | 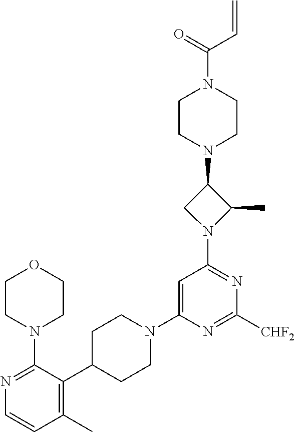 [SMILES: C=CC(=O)N1CCN([C@@H]2CN(c3cc(N4CCC(c5c(C)ccnc5N5CCOCC5)CC4)nc(C(F)F)n3)[C@@H]2C)CC1] | 1-(4-((2R,3R)-1-(2- (difluoromethyl)-6-(4-(4- methyl-2-(4-morpholinyl)-3- pyridinyl)-1-piperidinyl)-4- pyrimidinyl)-2-methyl-3- azetidinyl)-1-piperazinyl)-2- propen-1-one | E-9 | 597.4 |
| 2-584 | 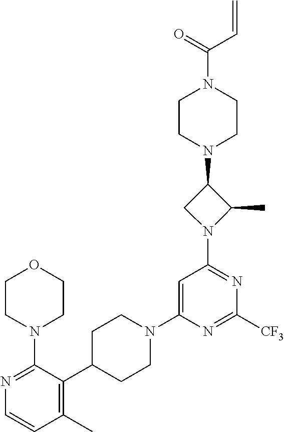 [SMILES: C=CC(=O)N1CCN([C@@H]2CN(c3cc(N4CCC(c5c(C)ccnc5N5CCOCC5)CC4)nc(C(F)(F)F)n3)[C@@H]2C)CC1] | 1-(4-((2R,3R)-2-methyl-1-(6- (4-(4-methyl-2-(4- morpholinyl)-3-pyridinyl)-1- piperidinyl)-2- (trifluoromethyl)-4- pyrimidinyl)-3-azetidinyl)-1- piperazinyl)-2-propen-1-one | E-1 | 615.3 |
| 2-585 | 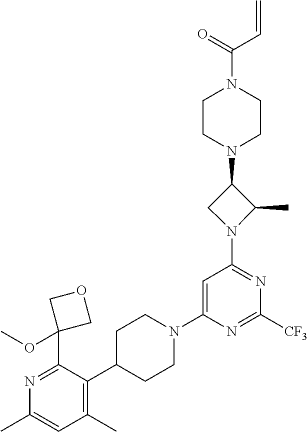 [SMILES: C=CC(=O)N1CCN([C@@H]2CN(c3cc(N4CCC(c5c(C)cc(C)nc5C5(OC)COC5)CC4)nc(C(F)(F)F)n3)[C@@H]2C)CC1] | 1-(4-((2R,3R)-1-(6-(4-(2-(3- methoxy-3-oxetanyl)-4,6- dimethyl-3-pyridinyl)-1- piperidinyl)-2- (trifluoromethyl)-4- pyrimidinyl)-2-methyl-3- azetidinyl)-1-piperazinyl)-2- propen-1-one | E-1 | 630.4 |
| 2-586 | 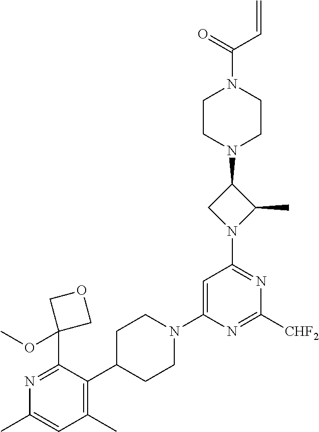 [SMILES: C=CC(=O)N1CCN([C@@H]2CN(c3cc(N4CCC(c5c(C)cc(C)nc5C5(OC)COC5)CC4)nc(C(F)F)n3)[C@@H]2C)CC1] | 1-(4-((2R,3R)-1-(2- (difluoromethyl)-6-(4-(2-(3- methoxy-3-oxetanyl)-4,6- dimethyl-3-pyridinyl)-1- piperidinyl)-4-pyrimidinyl)-2- methyl-3-azetidinyl)-1- piperazinyl)-2-propen-1-one | E-2 | 612.2 |
| 2-590 | 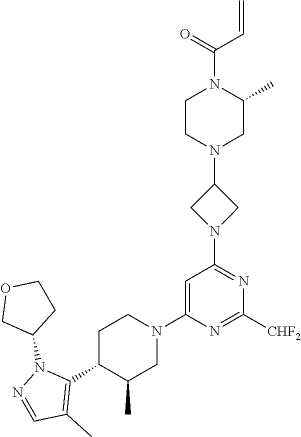 [SMILES: C=CC(=O)N1CCN(C2CN(c3cc(N4CC[C@@H](c5c(C)cnn5[C@H]5CCOC5)[C@H](C)C4)nc(C(F)F)n3)C2)C[C@H]1C] | 1-((2R)-4-(1-(2- (difluoromethyl)-6-((3S,4R)- 3-methyl-4-(4-methyl-1-((3S)- tetrahydro-3-furanyl)-1H- pyrazol-5-yl)-1-piperidinyl)-4- pyrimidinyl)-3-azetidinyl)-2- methyl-1-piperazinyl)-2- propen-1-one | E-14 | 585.3 |
| 2-591 | 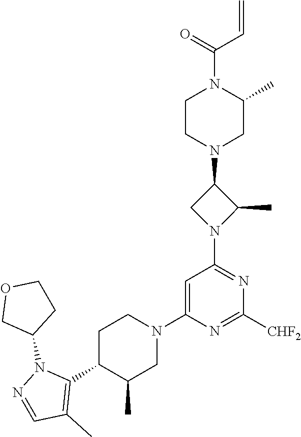 [SMILES: C=CC(=O)N1CCN([C@@H]2CN(c3cc(N4CC[C@@H](c5c(C)cnn5[C@H]5CCOC5)[C@H](C)C4)nc(C(F)F)n3)[C@@H]2C)C[C@H]1C] | 1-((2R)-4-((2R,3R)-1-(2- (difluoromethyl)-6-((3S,4R)- 3-methyl-4-(4-methyl-1-((3S)- tetrahydro-3-furanyl)-1H- pyrazol-5-yl)-1-piperidinyl)-4- pyrimidinyl)-2-methyl-3- azetidinyl)-2-methyl-1- piperazinyl)-2-propen-1-one | E-11 | 599.2 |
| 2-594 | 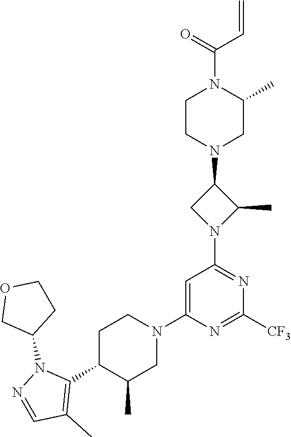 [SMILES: C=CC(=O)N1CCN([C@@H]2CN(c3cc(N4CC[C@@H](c5c(C)cnn5[C@H]5CCOC5)[C@H](C)C4)nc(C(F)(F)F)n3)[C@@H]2C)C[C@H]1C] | 1-((2R)-2-methyl-4-((2R,3R)- 2-methyl-1-(6-((3S,4R)-3- methyl-4-(4-methyl-1-((3S)- tetrahydro-3-furanyl)-1H- pyrazol-5-yl)-1-piperidinyl)-2- (trifluoromethyl)-4- pyrimidinyl)-3-azetidinyl)-1- piperazinyl)-2-propen-1-one | E-6 | 617.3 |
| 2-595 | 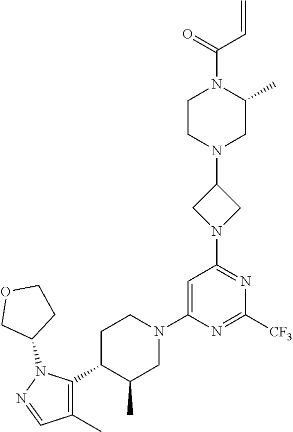 [SMILES: C=CC(=O)N1CCN(C2CN(c3cc(N4CC[C@@H](c5c(C)cnn5[C@H]5CCOC5)[C@H](C)C4)nc(C(F)(F)F)n3)C2)C[C@H]1C] | 1-((2R)-2-methyl-4-(1-(6- ((3S,4R)-3-methyl-4-(4- methyl-1-((3S)-tetrahydro-3- furanyl)-1H-pyrazol-5-yl)-1- piperidinyl)-2- (trifluoromethyl)-4- pyrimidinyl)-3-azetidinyl)-1- piperazinyl)-2-propen-1-one | E-4 | 603.5 |
| 2-596 | 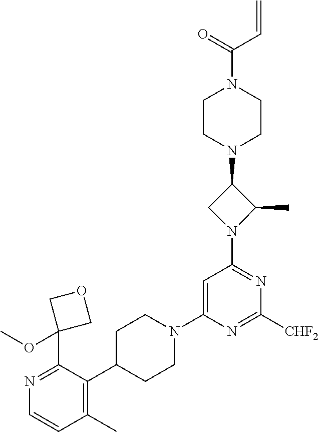 [SMILES: C=CC(=O)N1CCN([C@@H]2CN(c3cc(N4CCC(c5c(C)ccnc5C5(OC)COC5)CC4)nc(C(F)F)n3)[C@@H]2C)CC1] | 1-(4-((2R,3R)-1-(2- (difluoromethyl)-6-(4-(2-(3- methoxy-3-oxetanyl)-4- methyl-3-pyridinyl)-1- piperidinyl)-4-pyrimidinyl)-2- methyl-3-azetidinyl)-1- piperazinyl)-2-propen-1-one | E-9 | 598.3 |
| 2-597 | 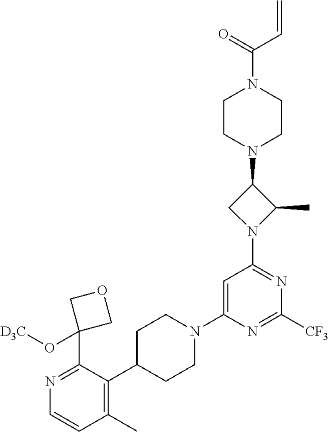 [SMILES: [2H]C([2H])([2H])OC1(c2nccc(C)c2C2CCN(c3cc(N4C[C@@H](N5CCN(C(=O)C=C)CC5)[C@H]4C)nc(C(F)(F)F)n3)CC2)COC1] | 1-(4-((2R,3R)-1-(6-(4-(2-(3- (methoxy-d3)oxetan-3-yl)-4- methylpyridin-3-yl)piperidin- 1-yl)-2- (trifluoromethyl)pyrimidin-4- yl)-2-methylazetidin-3- yl)piperazin-1-yl)prop-2-en-1- one | E-1 | 619.2 |
| 2-598 | 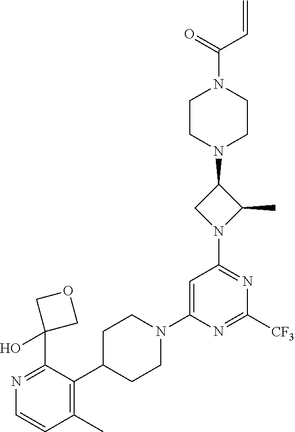 [SMILES: C=CC(=O)N1CCN([C@@H]2CN(c3cc(N4CCC(c5c(C)ccnc5C5(O)COC5)CC4)nc(C(F)(F)F)n3)[C@@H]2C)CC1] | 1-(4-((2R,3R)-1-(6-(4-(2-(3- hydroxy-3-oxetanyl)-4- methyl-3-pyridinyl)-1- piperidinyl)-2- (trifluoromethyl)-4- pyrimidinyl)-2-methyl-3- azetidinyl)-1-piperazinyl)-2- propen-1-one | E-1 | 602.2 |
| 2-599 | 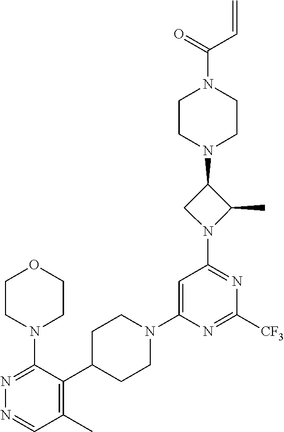 [SMILES: C=CC(=O)N1CCN([C@@H]2CN(c3cc(N4CCC(c5c(C)cnnc5N5CCOCC5)CC4)nc(C(F)(F)F)n3)[C@@H]2C)CC1] | 1-(4-((2R,3R)-2-methyl-1-(6- (4-(5-methyl-3-(4- morpholinyl)-4-pyridazinyl)- 1-piperidinyl)-2- (trifluoromethyl)-4- pyrimidinyl)-3-azetidinyl)-1- piperazinyl)-2-propen-1-one | E-1 | 616.3 |
| 2-600 | 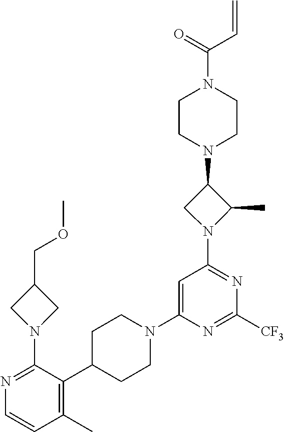 [SMILES: C=CC(=O)N1CCN([C@@H]2CN(c3cc(N4CCC(c5c(C)ccnc5N5CC(COC)C5)CC4)nc(C(F)(F)F)n3)[C@@H]2C)CC1] | 1-(4-((2R,3R)-1-(6-(4-(2-(3- (methoxymethyl)-1- azetidinyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-1 | 629.3 |
| 2-601 | 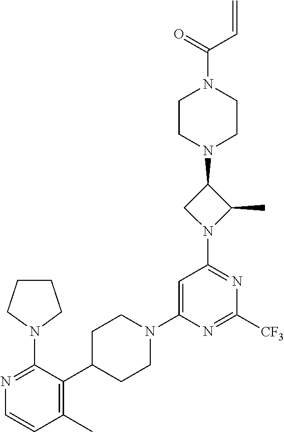 [SMILES: C=CC(=O)N1CCN([C@@H]2CN(c3cc(N4CCC(c5c(C)ccnc5N5CCCC5)CC4)nc(C(F)(F)F)n3)[C@@H]2C)CC1] | 1-(4-((2R,3R)-2-methyl-1-(6- (4-(4-methyl-2-(1- pyrrolidinyl)-3-pyridinyl)-1- piperidinyl)-2- (trifluoromethyl)-4- pyrimidinyl)-3-azetidinyl)-1- piperazinyl)-2-propen-1-one | E-1 | 599.3 |
| 2-602 | 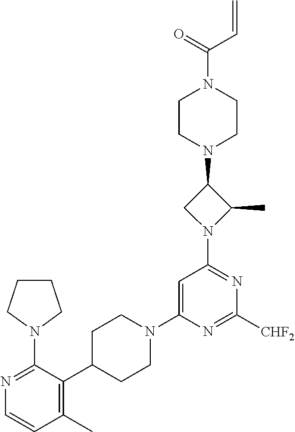 [SMILES: C=CC(=O)N1CCN([C@@H]2CN(c3cc(N4CCC(c5c(C)ccnc5N5CCCC5)CC4)nc(C(F)F)n3)[C@@H]2C)CC1] | 1-(4-((2R,3R)-1-(2- (difluoromethyl)-6-(4-(4- methyl-2-(1-pyrrolidinyl)-3- pyridinyl)-1-piperidinyl)-4- pyrimidinyl)-2-methyl-3- azetidinyl)-1-piperazinyl)-2- propen-1-one | E-9 | 581.3 |
| 2-603 | 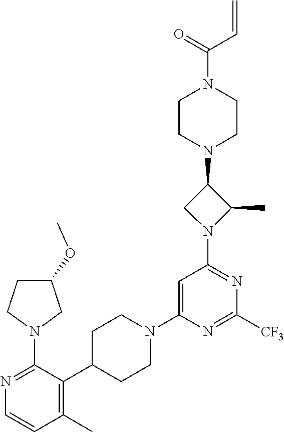 [SMILES: C=CC(=O)N1CCN([C@@H]2CN(c3cc(N4CCC(c5c(C)ccnc5N5CC[C@H](OC)C5)CC4)nc(C(F)(F)F)n3)[C@@H]2C)CC1] | 1-(4-((2R,3R)-1-(6-(4-(2- ((3S)-3-methoxy-1- pyrrolidinyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-8 | 629.3 |
| 2-604 | 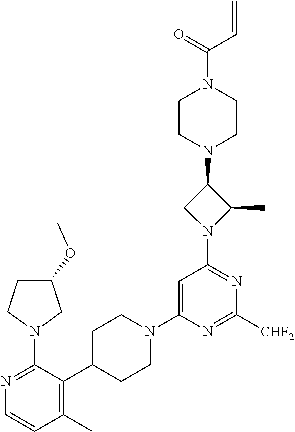 [SMILES: C=CC(=O)N1CCN([C@@H]2CN(c3cc(N4CCC(c5c(C)ccnc5N5CC[C@H](OC)C5)CC4)nc(C(F)F)n3)[C@@H]2C)CC1] | 1-(4-((2R,3R)-1-(2- (difluoromethyl)-6-(4-(2- ((3S)-3-methoxy-1- pyrrolidinyl)-4-methyl-3- pyridinyl)-1-piperidinyl)-4- pyrimidinyl)-2-methyl-3- azetidinyl)-1-piperazinyl)-2- propen-1-one | E-9 | 611.4 |
| 2-605 | 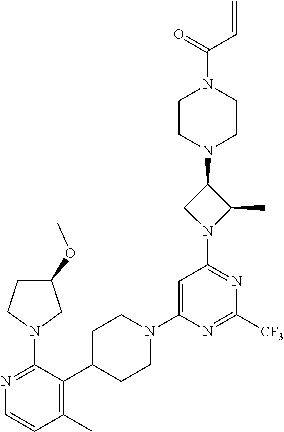 [SMILES: C=CC(=O)N1CCN([C@@H]2CN(c3cc(N4CCC(c5c(C)ccnc5N5CC[C@@H](OC)C5)CC4)nc(C(F)(F)F)n3)[C@@H]2C)CC1] | 1-(4-((2R,3R)-1-(6-(4-(2- ((3R)-3-methoxy-1- pyrrolidinyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-8 | 629.3 |
| 2-606 | 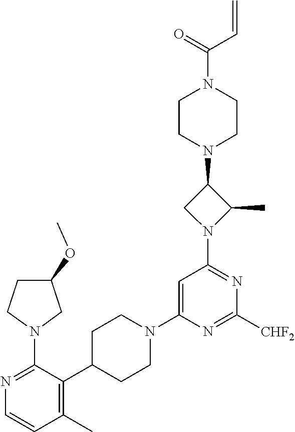 [SMILES: C=CC(=O)N1CCN([C@@H]2CN(c3cc(N4CCC(c5c(C)ccnc5N5CC[C@@H](OC)C5)CC4)nc(C(F)F)n3)[C@@H]2C)CC1] | 1-(4-((2R,3R)-1-(2- (difluoromethyl)-6-(4-(2- ((3R)-3-methoxy-1- pyrrolidinyl)-4-methyl-3- pyridinyl)-1-piperidinyl)-4- pyrimidinyl)-2-methyl-3- azetidinyl)-1-piperazinyl)-2- propen-1-one | E-9 | 611.3 |
| 2-607 | 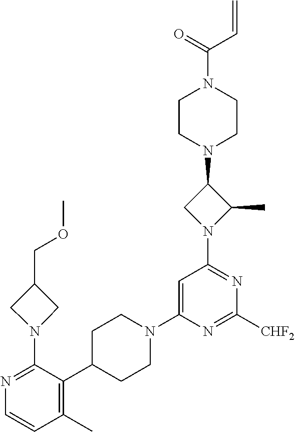 [SMILES: C=CC(=O)N1CCN([C@@H]2CN(c3cc(N4CCC(c5c(C)ccnc5N5CC(COC)C5)CC4)nc(C(F)F)n3)[C@@H]2C)CC1] | 1-(4-((2R,3R)-1-(2- (difluoromethyl)-6-(4-(2-(3- (methoxymethyl)-1- azetidinyl)-4-methyl-3- pyridinyl)-1-piperidinyl)-4- pyrimidinyl)-2-methyl-3- azetidinyl)-1-piperazinyl)-2- propen-1-one | E-2 | 611.4 |
| 2-608 | 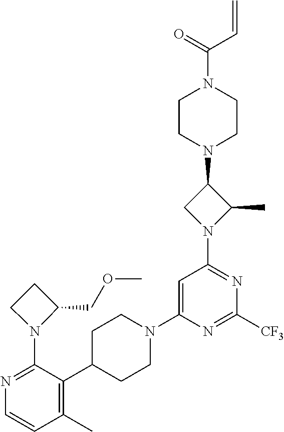 [SMILES: C=CC(=O)N1CCN([C@@H]2CN(c3cc(N4CCC(c5c(C)ccnc5N5CC[C@@H]5COC)CC4)nc(C(F)(F)F)n3)[C@@H]2C)CC1] | 1-(4-((2R,3R)-1-(6-(4-(2- ((2R)-2-(methoxymethyl)-1- azetidinyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-1 | 629.3 |
| 2-609 | 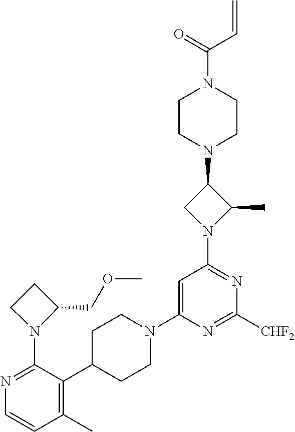 [SMILES: C=CC(=O)N1CCN([C@@H]2CN(c3cc(N4CCC(c5c(C)ccnc5N5CC[C@@H]5COC)CC4)nc(C(F)F)n3)[C@@H]2C)CC1] | 1-(4-((2R,3R)-1-(2- (difluoromethyl)-6-(4-(2- ((2R)-2-(methoxymethyl)-1- azetidinyl)-4-methyl-3- pyridinyl)-1-piperidinyl)-4- pyrimidinyl)-2-methyl-3- azetidinyl)-1-piperazinyl)-2- propen-1-one | E-2 | 611.4 |
| 2-610 | 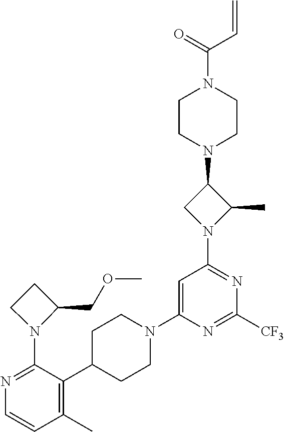 [SMILES: C=CC(=O)N1CCN([C@@H]2CN(c3cc(N4CCC(c5c(C)ccnc5N5CC[C@H]5COC)CC4)nc(C(F)(F)F)n3)[C@@H]2C)CC1] | 1-(4-((2R,3R)-1-(6-(4-(2- ((2S)-2-(methoxymethyl)-1- azetidinyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-1 | 629.3 |
| 2-611 | 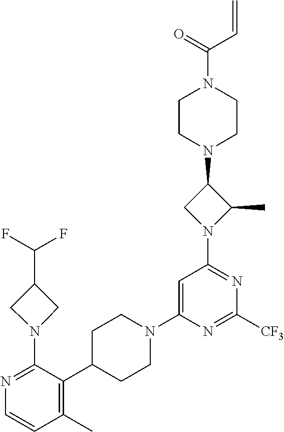 [SMILES: C=CC(=O)N1CCN([C@@H]2CN(c3cc(N4CCC(c5c(C)ccnc5N5CC(C(F)F)C5)CC4)nc(C(F)(F)F)n3)[C@@H]2C)CC1] | 1-(4-((2R,3R)-1-(6-(4-(2-(3- (difluoromethyl)-1- azetidinyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-8 | 635.3 |
| 2-612 | 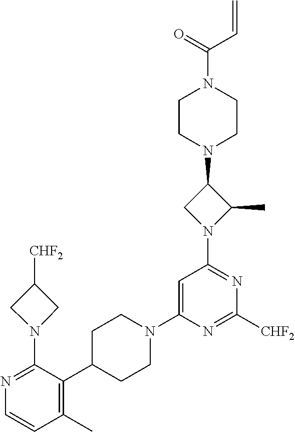 [SMILES: C=CC(=O)N1CCN([C@@H]2CN(c3cc(N4CCC(c5c(C)ccnc5N5CC(C(F)F)C5)CC4)nc(C(F)F)n3)[C@@H]2C)CC1] | 1-(4-((2R,3R)-1-(2- (difluoromethyl)-6-(4-(2-(3- (difluoromethyl)-1- azetidinyl)-4-methyl-3- pyridinyl)-1-piperidinyl)-4- pyrimidinyl)-2-methyl-3- azetidinyl)-1-piperazinyl)-2- propen-1-one | E-2 | 617.3 |
| 2-613 | 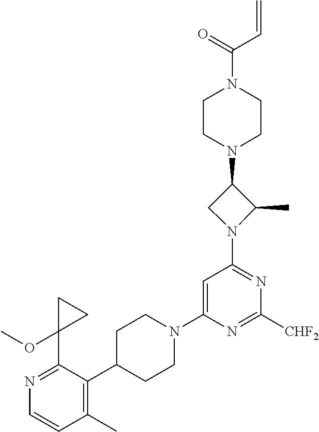 [SMILES: C=CC(=O)N1CCN([C@@H]2CN(c3cc(N4CCC(c5c(C)ccnc5C5(OC)CC5)CC4)nc(C(F)F)n3)[C@@H]2C)CC1] | 1-(4-((2R,3R)-1-(2- (difluoromethyl)-6-(4-(2-(1- methoxycyclopropyl)-4- methyl-3-pyridinyl)-1- piperidinyl)-4-pyrimidinyl)-2- methyl-3-azetidinyl)-1- piperazinyl)-2-propen-1-one | E-2 | 582.4 |
| 2-614 | 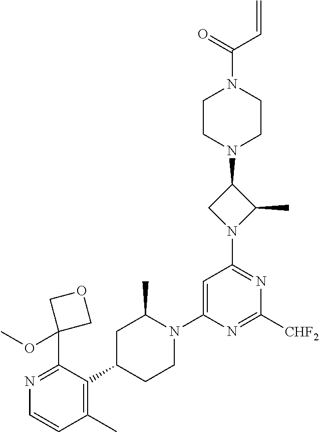 [SMILES: C=CC(=O)N1CCN([C@@H]2CN(c3cc(N4CC[C@H](c5c(C)ccnc5C5(OC)COC5)C[C@H]4C)nc(C(F)F)n3)[C@@H]2C)CC1] | 1-(4-((2R,3R)-1-(2- (difluoromethyl)-6-((2R,4S)- 4-(2-(3-methoxy-3-oxetanyl)- 4-methyl-3-pyridinyl)-2- methyl-1-piperidinyl)-4- pyrimidinyl)-2-methyl-3- azetidinyl)-1-piperazinyl)-2- propen-1-one | E-9 | 612.3 |
| 2-615 | 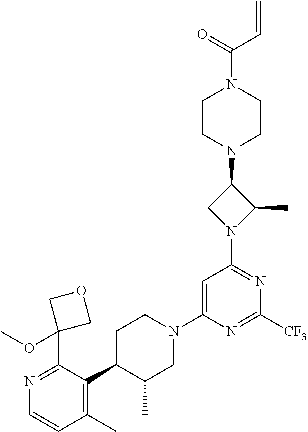 [SMILES: C=CC(=O)N1CCN([C@@H]2CN(c3cc(N4CC[C@H](c5c(C)ccnc5C5(OC)COC5)[C@@H](C)C4)nc(C(F)(F)F)n3)[C@@H]2C)CC1] | 1-(4-((2R,3R)-1-(6-((3R,4S)- 4-(2-(3-methoxy-3-oxetanyl)- 4-methyl-3-pyridinyl)-3- methyl-1-piperidinyl)-2- (trifluoromethyl)-4- pyrimidinyl)-2-methyl-3- azetidinyl)-1-piperazinyl)-2- propen-1-one | E-8 | 630.3 |
| 2-616 | 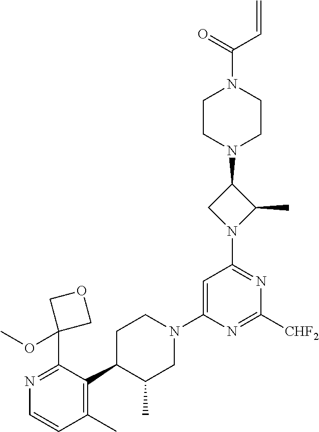 [SMILES: C=CC(=O)N1CCN([C@@H]2CN(c3cc(N4CC[C@H](c5c(C)ccnc5C5(OC)COC5)[C@@H](C)C4)nc(C(F)F)n3)[C@@H]2C)CC1] | 1-(4-((2R,3R)-1-(2- (difluoromethyl)-6-((3R,4S)- 4-(2-(3-methoxy-3-oxetanyl)- 4-methyl-3-pyridinyl)-3- methyl-1-piperidinyl)-4- pyrimidinyl)-2-methyl-3- azetidinyl)-1-piperazinyl)-2- propen-1-one | E-9 | 612.3 |
| 2-617 | 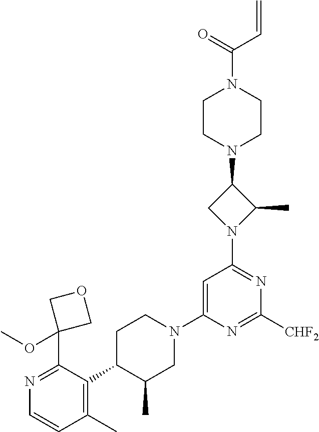 [SMILES: C=CC(=O)N1CCN([C@@H]2CN(c3cc(N4CC[C@@H](c5c(C)ccnc5C5(OC)COC5)[C@H](C)C4)nc(C(F)F)n3)[C@@H]2C)CC1] | 1-(4-((2R,3R)-1-(2- (difluoromethyl)-6-((3S,4R)- 4-(2-(3-methoxy-3-oxetanyl)- 4-methyl-3-pyridinyl)-3- methyl-1-piperidinyl)-4- pyrimidinyl)-2-methyl-3- azetidinyl)-1-piperazinyl)-2- propen-1-one | E-9 | 612.3 |
| 2-618 | 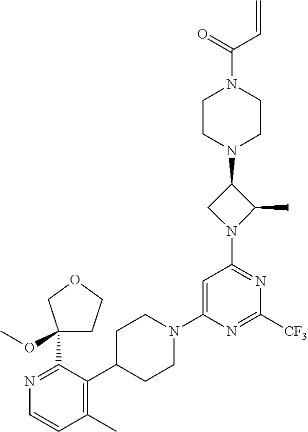 [SMILES: C=CC(=O)N1CCN([C@@H]2CN(c3cc(N4CCC(c5c(C)ccnc5[C@@]5(OC)CCOC5)CC4)nc(C(F)(F)F)n3)[C@@H]2C)CC1] | 1-(4-((2R,3R)-1-(6-(4-(2- ((3S)-3-methoxytetrahydro-3- furanyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-8 | 630.3 |
| | racemic mixture | | | |
| 2-619 | 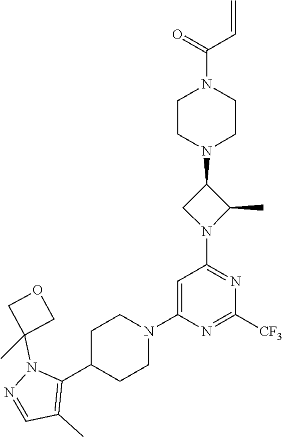 [SMILES: C=CC(=O)N1CCN([C@@H]2CN(c3cc(N4CCC(c5c(C)cnn5C5(C)COC5)CC4)nc(C(F)(F)F)n3)[C@@H]2C)CC1] | 1-(4-((2R,3R)-2-methyl-1-(6- (4-(4-methyl-1-(3-methyl-3- oxetanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 589.2 |
| 2-623 | 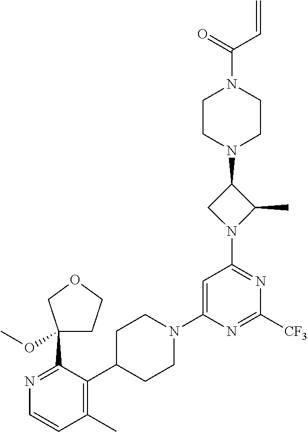 [SMILES: C=CC(=O)N1CCN([C@@H]2CN(c3cc(N4CCC(c5c(C)ccnc5[C@]5(OC)CCOC5)CC4)nc(C(F)(F)F)n3)[C@@H]2C)CC1] | 1-(4-((2R,3R)-1-(6-(4-(2- ((3R)-3-methoxytetrahydro-3- furanyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-1 | 630.3 |
| 2-624 | 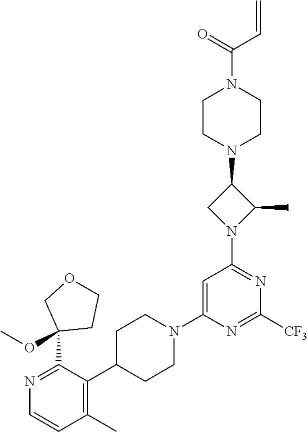 [SMILES: C=CC(=O)N1CCN([C@@H]2CN(c3cc(N4CCC(c5c(C)ccnc5[C@@]5(OC)CCOC5)CC4)nc(C(F)(F)F)n3)[C@@H]2C)CC1] | 1-(4-((2R,3R)-1-(6-(4-(2- ((3S)-3-methoxytetrahydro-3- furanyl)-4-methyl-3- pyridinyl)-1-piperidinyl)-2- (trifluoromethyl)-4- pyrimidinyl)-2-methyl-3- azetidinyl)-1-piperazinyl)-2- propen-1-one | E-1 | 630.3 |
| 2-625 | 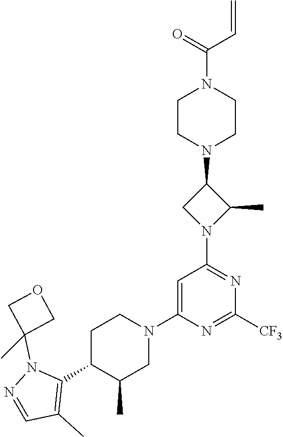 [SMILES: C=CC(=O)N1CCN([C@@H]2CN(c3cc(N4CC[C@@H](c5c(C)cnn5C5(C)COC5)[C@H](C)C4)nc(C(F)(F)F)n3)[C@@H]2C)CC1] | 1-(4-((2R,3R)-2-methyl-1-(6- ((3S,4R)-3-methyl-4-(4- methyl-1-(3-methyl-3- oxetanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 603.3 |
| 2-626 | 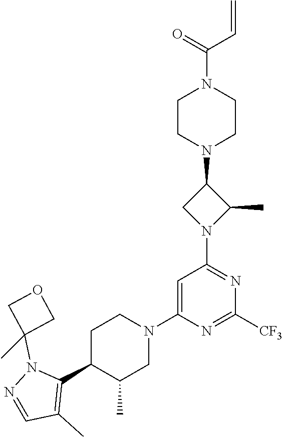 [SMILES: C=CC(=O)N1CCN([C@@H]2CN(c3cc(N4CC[C@H](c5c(C)cnn5C5(C)COC5)[C@@H](C)C4)nc(C(F)(F)F)n3)[C@@H]2C)CC1] | 1-(4-((2R,3R)-2-methyl-1-(6- ((3R,4S)-3-methyl-4-(4- methyl-1-(3-methyl-3- oxetanyl)-1H-pyrazol-5-yl)-1- piperidinyl)-2- (trifluoromethyl)-4- pyrimidinyl)-3-azetidinyl)-1- piperazinyl)-2-propen-1-one | E-1 | 603.3 |
| 2-627 | 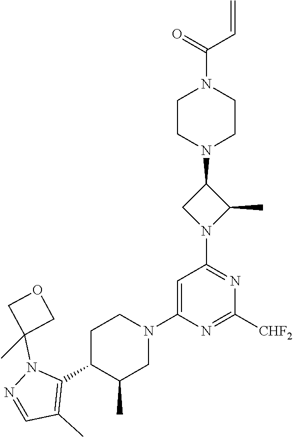 [SMILES: C=CC(=O)N1CCN([C@@H]2CN(c3cc(N4CC[C@@H](c5c(C)cnn5C5(C)COC5)[C@H](C)C4)nc(C(F)F)n3)[C@@H]2C)CC1] | 1-(4-((2R,3R)-1-(2- (difluoromethyl)-6-((3S,4R)- 3-methyl-4-(4-methyl-1-(3- methyl-3-oxetanyl)-1H- pyrazol-5-yl)-1-piperidinyl)-4- pyrimidinyl)-2-methyl-3- azetidinyl)-1-piperazinyl)-2- propen-1-one | E-2 | 585.3 |
| 2-628 | 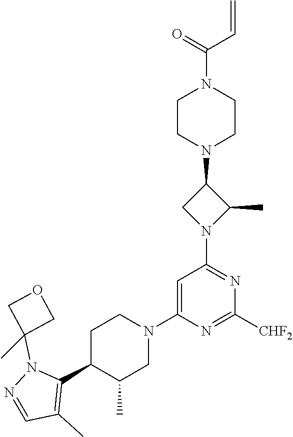 [SMILES: C=CC(=O)N1CCN([C@@H]2CN(c3cc(N4CC[C@H](c5c(C)cnn5C5(C)COC5)[C@@H](C)C4)nc(C(F)F)n3)[C@@H]2C)CC1] | 1-(4-((2R,3R)-1-(2- (difluoromethyl)-6-((3R,4S)- 3-methyl-4-(4-methyl-1-(3- methyl-3-oxetanyl)-1H- pyrazol-5-yl)-1-piperidinyl)-4- pyrimidinyl)-2-methyl-3- azetidinyl)-1-piperazinyl)-2- propen-1-one | E-2 | 585.3 |
| 2-640 | 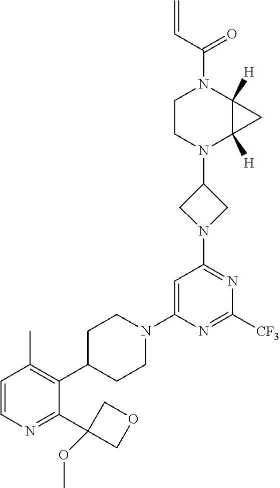 [SMILES: C=CC(=O)N1CCN(C2CN(c3cc(N4CCC(c5c(C)ccnc5C5(OC)COC5)CC4)nc(C(F)(F)F)n3)C2)[C@H]2C[C@H]21] | 1-((1R,6S)-5-(1-(6-(4-(2-(3- methoxy-3-oxetanyl)-4- methyl-3-pyridinyl)-1- piperidinyl)-2- (trifluoromethyl)-4- pyrimidinyl)-3-azetidinyl)- 2,5-diazabicyclo[4.1.0]heptan- 2-yl)-2-propen-1-one | E-15 | 614.3 |

| | | | Racemic SM \| |
|---|---|---|---|
| Ex.# | Chemical Structure | Name | separation condition |
| 2-009-1 | 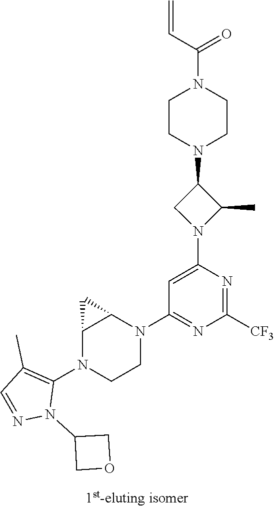 [SMILES: C=CC(=O)N1CCN([C@@H]2CN(c3cc(N4CCN(c5c(C)cnn5C5COC5)[C@@H]5C[C@@H]54)nc(C(F)(F)F)n3)[C@@H]2C)CC1] | 1-(4-((2R,3R)-2-methyl-1-(6- ((1S,6R)-5-(4-methyl-1- (oxetan-3-yl)-1H-pyrazol-5- yl)-2,5- diazabicyclo[4.1.0]heptan-2- yl)-2- (trifluoromethyl)pyrimidin-4- yl)azetidin-3-yl)piperazin-1- yl)prop-2-en-1-one | 9 \| SFC (Chiralpak AD, 21 x 250 mm 5 μm, column with a mobile phase of 35% MeOH using a flow rate of 100 mL/min) |
| 2-009-2 | 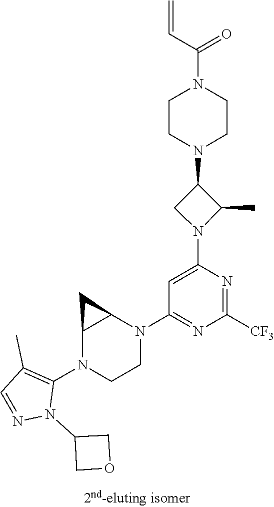 [SMILES: C=CC(=O)N1CCN([C@@H]2CN(c3cc(N4CCN(c5c(C)cnn5C5COC5)[C@H]5C[C@H]54)nc(C(F)(F)F)n3)[C@@H]2C)CC1] | 1-(4-((2R,3R)-2-methyl-1-(6- (1R,6S)-5-(4-methyl-1- (oxetan-3-yl)-1H-pyrazol-5- yl)-2.5- diazabicyclo[4.1.0]heptan-2- yl)-2- (trifluoromethyl)pyrimidin-4- yl)azetidin-3-yl)piperazin-1- yl)prop-2-en-1-one | 9 \| SFC (Chiralpak AD, 21 x 250 mm 5 μm, column with a mobile phase of 35% MeOH using a flow rate of 100 mL/min) |
| 2-025-1 | 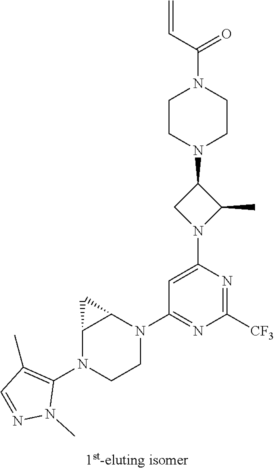 [SMILES: C=CC(=O)N1CCN([C@@H]2CN(c3cc(N4CCN(c5c(C)cnn5C)[C@@H]5C[C@@H]54)nc(C(F)(F)F)n3)[C@@H]2C)CC1] | 1-(4-((2R,3R)-1-(6-((1S,6R)-5- (1,4-dimethyl-1H-pyrazol-5- yl)-2,5- diazabicyclo[4.1.0]heptan-2- yl)-2- (trifluoromethyl)pyrimidin-4- yl)-2-methylazetidin-3- yl)piperazin-1-yl)prop-2-en-1- one | 25 \| SFC (ChiralPak AD, 2 x 25 cm 5 μm column with a mobile phase of 40% iPrOH using a flow rate of 80 mL/min) |
| 2-025-2 | 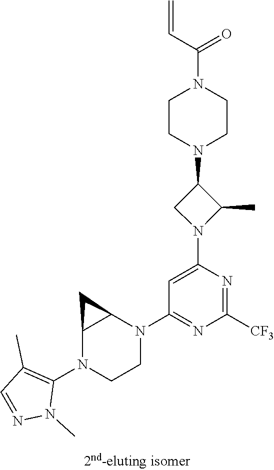 [SMILES: C=CC(=O)N1CCN([C@@H]2CN(c3cc(N4CCN(c5c(C)cnn5C)[C@H]5C[C@H]54)nc(C(F)(F)F)n3)[C@@H]2C)CC1] | 1-(4-((2R,3R)-1-(6-((1R,6S)-5- (1,4-dimethyl-1H-pyrazol-5- yl)-2,5- diazabicyclo[4.1.0]heptan-2- yl)-2- (trifluoromethyl)pyrimidin-4- yl)-2-methylazetidin-3- yl)piperazin-1-yl)prop-2-en-1- one | 25 \| SFC (ChiralPak AD, 2 x 25 cm 5 μm column with a mobile phase of 40% iPrOH using a flow rate of 80 mL/min) |
| 2-030-1 | 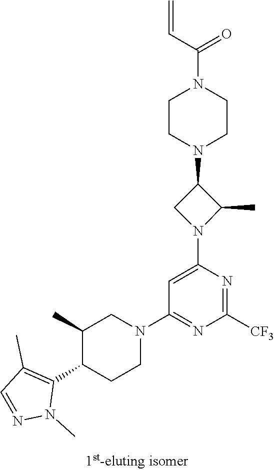 [SMILES: C=CC(=O)N1CCN([C@@H]2CN(c3cc(N4CC[C@H](c5c(C)cnn5C)[C@@H](C)C4)nc(C(F)(F)F)n3)[C@@H]2C)CC1] | 1-(4-((2R,3R)-1-(6-((3R,4S)-4- (1,4-dimethyl-1H-pyrazol-5- yl)-3-methylpiperidin-1-yl)-2- (trifluoromethyl)pyrimidin-4- yl)-2-methylazetidin-3- yl)piperazin-1-yl)prop-2-en-1- one | 30 \| SFC (Chiralcel OX column (21 x 150 mm, 5 μm) with a mobile phase of 70% CO₂ and 30% MeOH using a flow rate of 100 mL/min) |
| 2-030-2 | 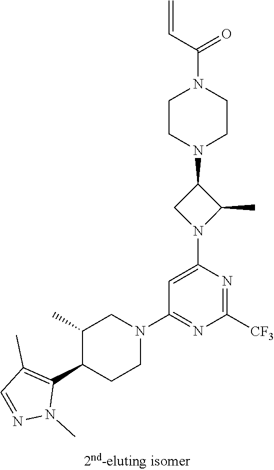 [SMILES: C=CC(=O)N1CCN([C@@H]2CN(c3cc(N4CC[C@@H](c5c(C)cnn5C)[C@H](C)C4)nc(C(F)(F)F)n3)[C@@H]2C)CC1] | 1-(4-((2R,3R)-1-(6-((3S,4R)-4- (1,4-dimethyl-1H-pyrazol-5- yl)-3-methylpiperidin-1-yl)-2- (trifluoromethyl)pyrimidin-4- yl)-2-methylazetidin-3- yl)piperazin-1-yl)prop-2-en-1- one | 30 \| SFC (Chiralcel OX column (21 x 150 mm, 5 μm) with a mobile phase of 70% CO₂ and 30% MeOH using a flow rate of 100 mL/min) |
| 2-032-1 | 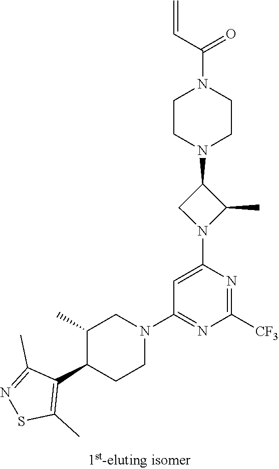 [SMILES: C=CC(=O)N1CCN([C@@H]2CN(c3cc(N4CC[C@@H](c5c(C)nsc5C)[C@H](C)C4)nc(C(F)(F)F)n3)[C@@H]2C)CC1] | 1-(4-((2R,3R)-1-(6-((3S,4R)-4- (3,5-dimethylisothiazol-4-yl)- 3-methylpiperidin-1-yl)-2- (trifluoromethyl)pyrimidin-4- yl)-2-methylazctidin-3- yl)piperazin-1-yl)prop-2-en-1- one | SFC \| 32 (Chromega Chiral CC4, 2 x 25 cm 5 μm column with a mobile phase of 65% liquid CO₂ and 35% MeOH using a flow rate of 80 mL/min) |
| 2-032-2 | 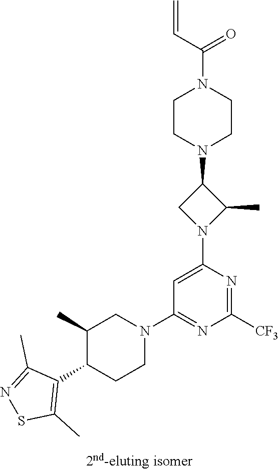 [SMILES: C=CC(=O)N1CCN([C@@H]2CN(c3cc(N4CC[C@H](c5c(C)nsc5C)[C@@H](C)C4)nc(C(F)(F)F)n3)[C@@H]2C)CC1] | 1-(4-((2R,3R)-1-(6-((3R,4S)-4- (3,5-dimethylisothiazol-4-yl)- 3-methylpiperidin-1-yl)-2- (trifluoromethyl)pyrimidin-4- yl)-2-methylazetidin-3- yl)piperazin-1-yl)prop-2-en-1- one | SFC \| 32 (Chromega Chiral CC4, 2 x 25 cm 5 μm column with a mobile phase of 65% liquid CO₂ and 35% MeOH using a flow rate of 80 mL/min) |
| 2-036-1 | 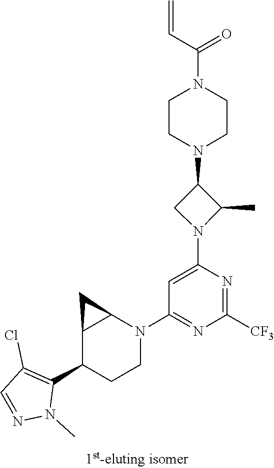 [SMILES: C=CC(=O)N1CCN([C@@H]2CN(c3cc(N4CC[C@@H](c5c(Cl)cnn5C)[C@H]5C[C@H]54)nc(C(F)(F)F)n3)[C@@H]2C)CC1] | 1-(4-((2R,3R)-1-(6- ((1R,5R,6R)-5-(4-chloro-1- methyl-1H-pyrazol-5-yl)-2- azabicyclo[4.1.0]heptan-2-yl)- 2-(trifluoromethyl)pyrimidin- 4-yl)-2-methylazetidin-3- yl)piperazin-1-yl)prop-2-en-1- one | SFC \| 36 (Chiralcel OX. 2 x 25 cm 5 μm column with a mobile phase of 55% liquid CO₂ and 45% iPrOH using a flow rate of 70 mL/min) |
| 2-036-2 | 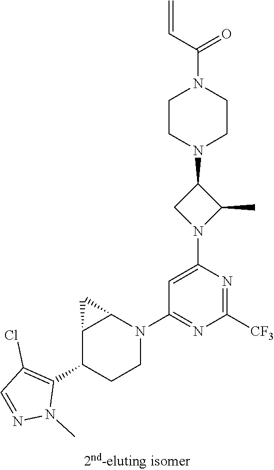 [SMILES: C=CC(=O)N1CCN([C@@H]2CN(c3cc(N4CC[C@H](c5c(Cl)cnn5C)[C@@H]5C[C@@H]54)nc(C(F)(F)F)n3)[C@@H]2C)CC1] | 1-(4-((2R,3R)-1-(6- ((1S,5S,6S)-5-(4-chloro-1- methyl-1H-pyrazol-5-yl)-2- azabicyclo[4.1.0]heptan-2-yl)- 2-(trifluoromethyl)pyrimidin- 4-yl)-2-methylazetidin-3- yl)piperazin-1-yl)prop-2-en-1- one | SFC \| 36 (Chiralcel OX, 2 x 25 cm 5 μm column with a mobile phase of 55% liquid CO₂ and 45% iPrOH using a flow rate of 70 mL/min) |
| 2-038-1 | 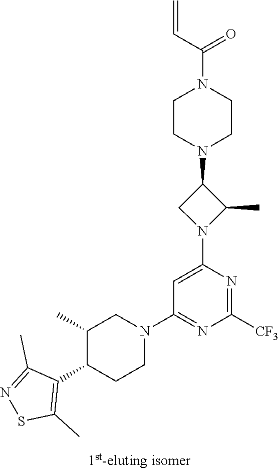 [SMILES: C=CC(=O)N1CCN([C@@H]2CN(c3cc(N4CC[C@H](c5c(C)nsc5C)[C@H](C)C4)nc(C(F)(F)F)n3)[C@@H]2C)CC1] | 1-(4-((2R,3R)-1-(6-((3S,4S)-4- (3,5-dimethylisothiazol-4-yl)- 3-methylpiperidin-1-yl)-2- (trifluoromethyl)pyrimidin-4- yl)-2-methylazetidin-3- yl)piperazin-1-yl)prop-2-en-1- one | SFC \| 38 (Chiralcel OX, 2 x 25 cm 5 μm column with a mobile phase of 55% liquid CO₂ and 45% iPrOH using a flow rate of 80 mL/min) |
| 2-038-2 | 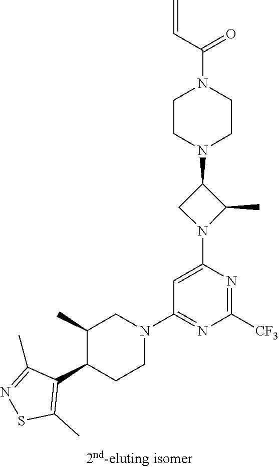 [SMILES: C=CC(=O)N1CCN([C@@H]2CN(c3cc(N4CC[C@@H](c5c(C)nsc5C)[C@@H](C)C4)nc(C(F)(F)F)n3)[C@@H]2C)CC1] | 1-(4-((2R,3R)-1-(6-((3R,4R)-4- (3,5-dimethylisothiazol-4-yl)- 3-methylpiperidin-1-yl)-2- (trifluoromethyl)pyrimidin-4- yl)-2-methylazetidin-3- yl)piperazin-1-yl)prop-2-en-1- one | SFC \| 38 (Chiralcel OX, 2 x 25 cm 5 μm column with a mobile phase of 55% liquid CO₂ and 45% IPA using a flow rate of 80 mL/min) |
| 2-042-1 | 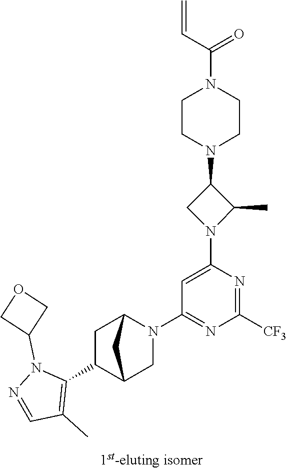 | 1-(4-((2R,3R)-2-methyl-1-(6- ((1R,4S,5R)-5-(4-methyl-1- (oxetan-3-yl)-1H-pyrazol-5- yl)-2-azabicyclo[2.2.1]heptan- 2-yl)-2- (trifluoromethyl)pyrimidin-4- yl)azetidin-3-yl)piperazin-1- yl)prop-2-en-1-one | SFC \| 42 (Chiralpak IC, 21 x 150 mm 5 μm, column with a mobile phase of 35% liquid CO₂ and 65% MeOH with 0.2% triethy lamine using a flow rate of 80 mL/min) |
| 2-042-2 | 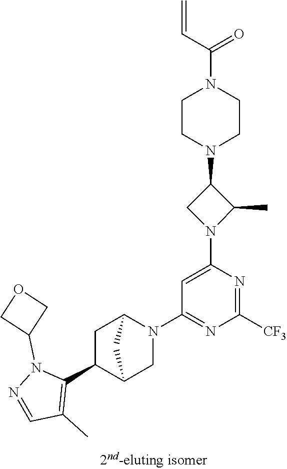 [SMILES: C=CC(=O)N1CCN([C@@H]2CN(c3cc(N4C[C@@H]5C[C@H]4C[C@@H]5c4c(C)cnn4C4COC4)nc(C(F)(F)F)n3)[C@@H]2C)CC1] | 1-(4-((2R,3R)-2-methyl-1-(6- ((1S,4R,5S)-5-(4-methyl-1- (oxetan-3-yl)-1H-pyrazol-5- yl)-2-azabicyclo[2.2.1]heptan- 2-yl)-2- (trifluoromethyl)pyrimidin-4- yl)azetidin-3-yl)piperazin-1- yl)prop-2-en-1-one | SFC \| 42 (Chiralpak IC, 21 x 150 mm 5 μm, column with a mobile phase of 35% liquid CO₂ and 65% MeOH with 0.2% triethylamine using a flow rate of 80 mL/min) |
| 2-084-1 | 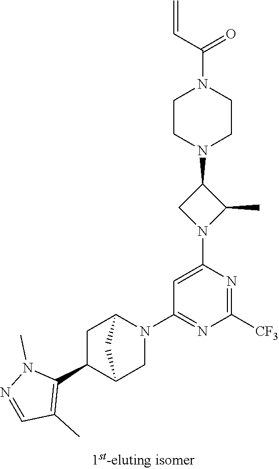 [SMILES: C=CC(=O)N1CCN([C@@H]2CN(c3cc(N4C[C@@H]5C[C@H]4C[C@@H]5c4c(C)cnn4C)nc(C(F)(F)F)n3)[C@@H]2C)CC1] | 1-(4-((2R,3R)-1-(6- ((1S,4R,5S)-5-(1,4-dimethyl- 1H-pyrazol-5-yl)-2- azabicyclo[2.2.1]heptan-2-yl)- 2-(trifluoromethyl)pyrimidin- 4-yl)-2-methylazetidin-3- yl)piperazin-1-yl)prop-2-en-1- one | SFC \| 84 (Chiralpak IF, 21 x 250 mm 5 μm. column with a mobile phase of 70% liquid CO₂ and 30% MeOH using a flow rate of 120 mL/min) |
| 2-084-2 | 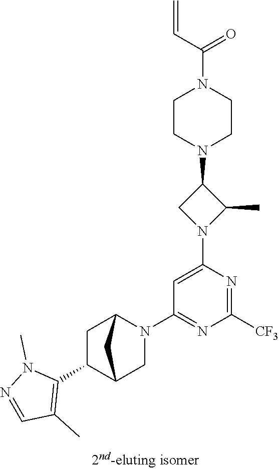 [SMILES: C=CC(=O)N1CCN([C@@H]2CN(c3cc(N4C[C@H]5C[C@@H]4C[C@H]5c4c(C)cnn4C)nc(C(F)(F)F)n3)[C@@H]2C)CC1] | 1-(4-((2R,3R)-1-(6- ((1R,4S,5R)-5-(1,4-dimethyl- 1H-pyrazol-5-yl)-2- azabicyclo[2.2.1]heptan-2-yl)- 2-(trifluoromethyl)pyrimidin- 4-yl)-2-methylazetidin-3- yl)piperazin-1-yl)prop-2-en-1- one | SFC \| 84 (Chiralpak IF, 21 x 250 mm 5 μm, column with a mobile phase of 70% liquid CO₂ and 30% MeOH using a flow rate of 120 mL/min) |
| 2-101-1 | 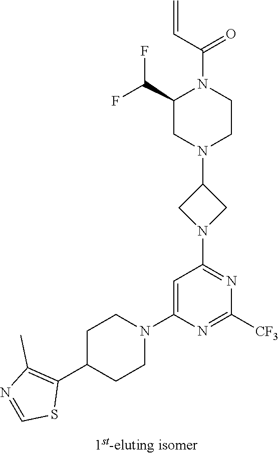 [SMILES: C=CC(=O)N1CCN(C2CN(c3cc(N4CCC(c5scnc5C)CC4)nc(C(F)(F)F)n3)C2)C[C@H]1C(F)F] | (S)-1-(2-(difluoromethyl)-4- (1-(6-(4-(4-methylthiazol-5- yl)piperidin-1-yl)-2- (trifluoromethyl)pyrimidin-4- yl)azetidin-3-yl)piperazin-1- yl)prop-2-en-1-one | SFC \| 101 (Chiralpak IC, 21 x 150 mm 5 μm, column with a mobile phase of 50% MeOH in liquid CO₂ using a flow rate of 120 mL/min) |
| 2-101-2 | 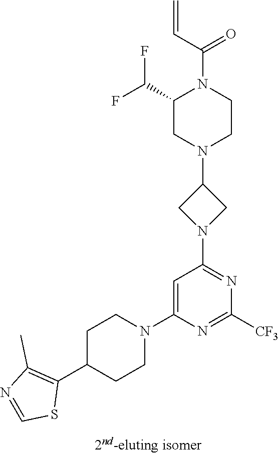 [SMILES: C=CC(=O)N1CCN(C2CN(c3cc(N4CCC(c5scnc5C)CC4)nc(C(F)(F)F)n3)C2)C[C@@H]1C(F)F] | (R)-1-(2-(difluoromethyl)-4- (1-(6-(4-(4-methylthiazol-5- yl)piperidin-1-yl)-2- (trifluoromethyl)pyrimidin-4- ylazetidin-3-yl)piperazin-1- yl)prop-2-en-1-one | SFC \| 101 (Chiralpak IC, 21 x 150 mm 5 μm, column with a mobile phase of 50% MeOH in liquid CO₂ using a flow rate of 120 mL/min) |
| 2-391-1 | 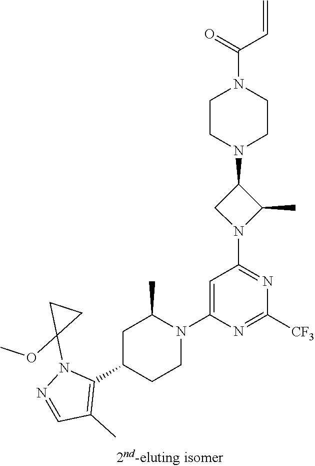 [SMILES: C=CC(=O)N1CCN([C@@H]2CN(c3cc(N4CC[C@H](c5c(C)cnn5C5(OC)CC5)C[C@H]4C)nc(C(F)(F)F)n3)[C@@H]2C)CC1] | 1-(4-((2R,3R)-1-(6-((2R,4S)- 4-(1-(1-methoxycyclopropyl)- 4-methyl-1H-pyrazol-5-yl)-2- methyl-1-piperidinyl)-2- (trifluoromethyl)-4- pyrimidinyl)-2-methyl-3- azetidinyl)-1-piperazinyl)-2- propen-1-one | SFC \| 391 (Chiralpak ID, 21 x 250 mm 5 μm, column with a mobile phase of 35% MeOH in liquid CO₂ using a flow rate of 80 mL/min) |
| 2-394-1 | 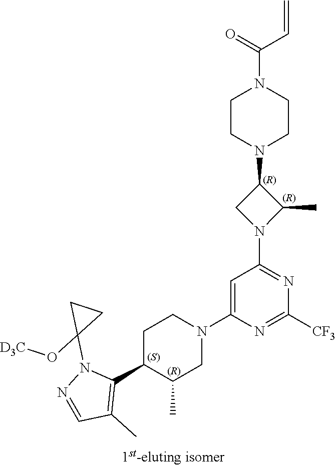 | 1-(4-((2R,3R)-1-(6-((3R,4S)- 4-(1-(1-(methoxy- d₃)cyclopropyl)-4-methyl-1H- pyrazol-5-yl)-3- methylpiperidin-1-yl)-2- (trifluoromethyl)pyrimidin-4- yl)-2-methylazetidin-3- yl)piperazin-1-yl)prop-2-en-1- one | SFC \| 394 Chiralcel OJ, 21 x 250 mm 5 μm, column with a mobile phase of 15% MeOH using a flowrate of 150 mL/min |
| 2-394-2 | 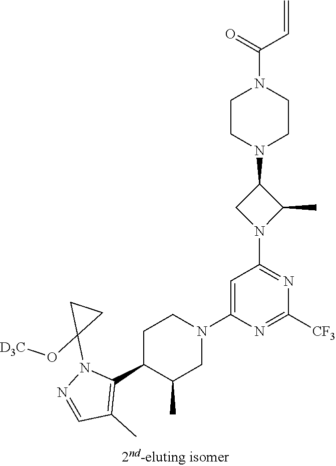 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)(F)F)n3)C[C@H]2C)CC1] | 1-(4-((2R,3R)-1-(6-((3S,4S)-4- (1-(1-(methoxy- d₃)cyclopropyl)-4-methyl-1H- pyrazol-5-yl)-3- methylpiperidin-1-yl)-2- (trifluoromethyl)pyrimidin-4- yl)-2-methylazetidin-3- yl)piperazin-1-yl)prop-2-en-1- one | SFC \| 394 Chiralcel OJ, 21 x 250 mm 5 μm, column with a mobile phase of 15% McOH using a flowrate of 150 mL/min |
| 2-394-3 | 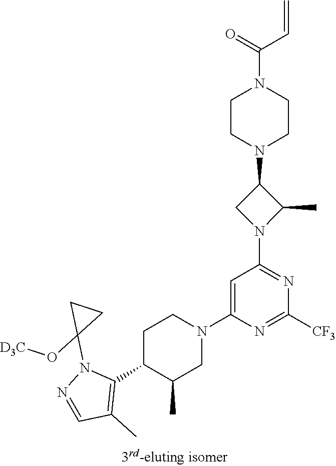 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)(F)F)n3)C[C@H]2C)CC1] | 1-(4-((2R,3R)-1-(6-((3S,4R)- 4-(1-(1-(methoxy- d₃)cyclopropyl)-4-mcthyl-1H- pyrazol-5-yl)-3- methylpiperidin-1-yl)-2- (trifluoromethyl)pyrimidin-4- yl)-2-methylazetidin-3- yl)piperazin-1-yl)prop-2-en-1- one | SFC \| 394 Chiralcel OJ, 21 x 250 mm 5 μm, column with a mobile phase of 15% MeOH using a flowrate of 150 mL/min |
| 2-394-4 | 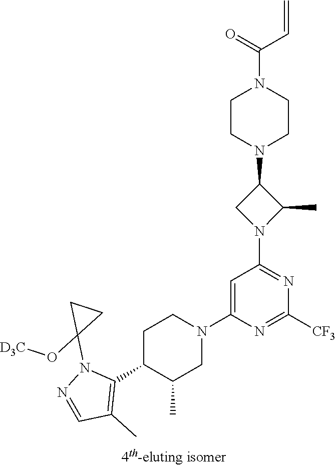 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)(F)F)n3)C[C@@H]2C)CC1] | 1-(4-((2R,3R)-1-(6-((3R,4R)- 4-(1-(1-(methoxy- d₃)cyclopropyl)-4-methyl-1H- pyrazol-5-yl)-3- methylpiperidin-1-yl)-2- (trifluoromethyl)pyrimidin-4- yl)-2-methylazetidin-3- yl)piperazin-1-yl)prop-2-en-1- one | SFC \| 394 Chiralcel OJ, 21 x 250 mm 5 μm, column with a mobile phase of 15% MeOH using a flowrate of 150 mL/min |
| 2-395-1 | 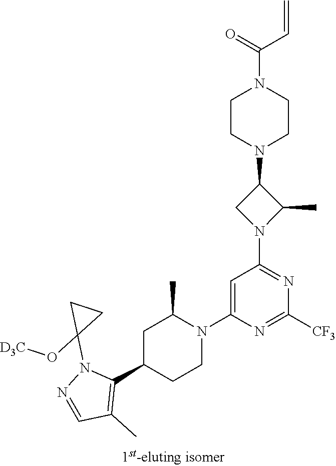 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)(F)F)n3)[C@H](C)C2)CC1] | 1-(4-((2R.3R)-1-(6-((2R,4R)- 4-(1-(1-(methoxy- d₃)cyclopropyl)-4-methyl-1H- pyrazol-5-yl)-3- methylpiperidin-1-yl)-2- (trifluoromethyl)pyrimidin-4- yl)-2-methylazetidin-3- yl)piperazin-1-yl)prop-2-en-1- one | SFC \| 395 ChiralPak ID, 2 x 25 cm 5 μm column with a mobile phase of 35% MeOH using a flowrate of 80 mL/min |
| 2-395-2 | 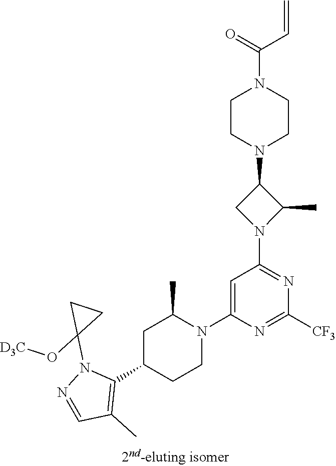 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)(F)F)n3)[C@H](C)C2)CC1] | 1-(4-((2R,3R)-1-(6-((2R,4S)- 4-(1-(1-(methoxy- d₃)cyclopropyl)-4-methyl-1H- pyrazol-5-yl)-3- methylpiperidin-1-yl)-2- (trifluoromethyl)pyrimidin-4- yl)-2-methylazetidin-3- yl)piperazin-1-yl)prop-2-en-1- one | SFC \| 395 ChiralPak ID, 2 x 25 cm 5 μm column with a mobile phase of 35% MeOH using a flowrate of 80 mL/min |
| 2-397-1 | 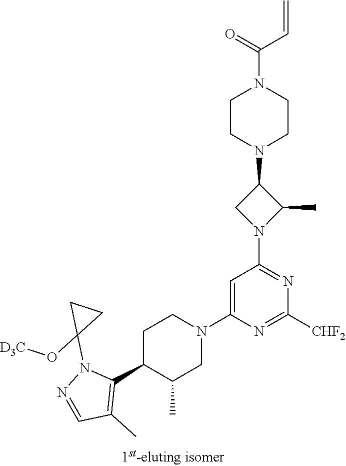 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)F)n3)C[C@@H]2C)CC1] | 1-(4-((2R,3R)-1-(2- (difluoromethyl)-6-((3R,4S)- 4-(1-(1-(methoxy- d₃)cyclopropyl)-4-methyl-1H- pyrazol-5-yl)-3- methylpiperidin-1- yl)pyrimidin-4-yl)-2- methylazetidin-3-yl)piperazin- 1-yl)prop-2-en-1-one | SFC \| 397 Chiralcel OJ, 21 x 250 mm 5 μm, column with a mobile phase of 15% MeOH using a flowrate of 150 mL/min |
| 2-397-2 | 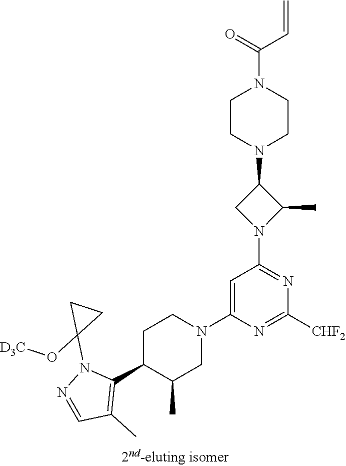 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)F)n3)C[C@H]2C)CC1] | 1-(4-((2R,3R)-1-(2- (difluoromethyl)-6-((3S,4S)-4- (1-(1-(methoxy- d₃)cyclopropyl)-4-methyl-1H- pyrazol-5-yl)-3- methylpiperidin-1- yl)pyrimidin-4-yl)-2- methylazetidin-3-yl)piperazin- 1-yl)prop-2-en-1-one | SFC \| 397 Chiralcel OJ, 21 x 250 mm 5 μm, column with a mobile phase of 15% MeOH using a flowrate of 150 mL/min |
| 2-397-3 | 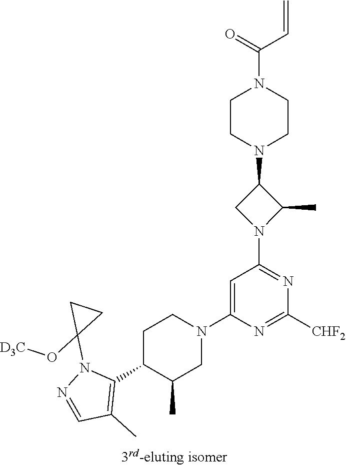 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)F)n3)C[C@H]2C)CC1] | 1-(4-((2R,3R)-1-(2- (difluoromethyl)-6-((3S,4R)- 4-(1-(1-(methoxy- d₃)cyclopropyl)-4-methyl-1H- pyrazol-5-yl)-3- methylpipcridin-1- yl)pyrimidin-4-yl)-2- methylazetidin-3-yl)piperazin- 1-yl)prop-2-en-1-one | SFC \| 397 Chiralcel OJ, 21 x 250 mm 5 μm, column with a mobile phase of 15% MeOH using a flowrate of 150 mL/min |
| 2-397-4 | 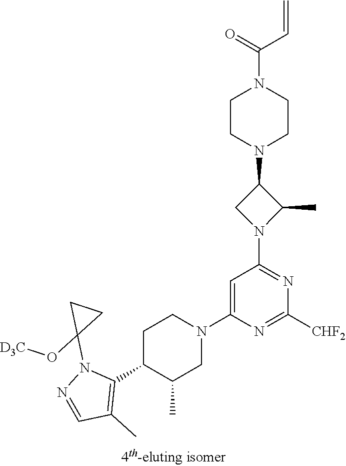 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)F)n3)C[C@@H]2C)CC1] | 1-(4-((2R,3R)-1-(2- (difluoromethyl)-6-((3R,4R)- 4-(1-(1-(methoxy- d₃)cyclopropyl)-4-methyl-1H- pyrazol-5-yl)-3- methylpiperidin-1- yl)pyrimidin-4-yl)-2- methylazetidin-3-yl)piperazin- 1-yl)prop-2-en-1-one | SFC \| 397 Chiralcel OJ, 21 x 250 mm 5 μm, column with a mobile phase of 15% MeOH using a flowrate of 150 mL/min |
| 2-400-1 | 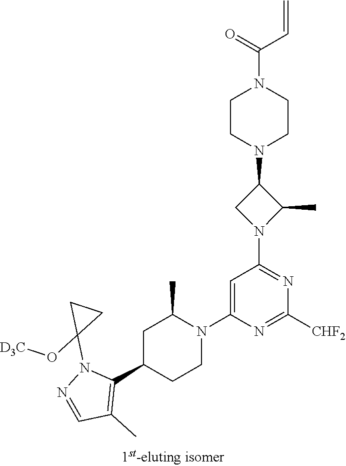 [SMILES: [2H]C([2H])([2H])OC1(n2ncc(C)c2[C@@H]2CCN(c3cc(N4C[C@@H](N5CCN(C(=O)C=C)CC5)[C@H]4C)nc(C(F)F)n3)[C@H](C)C2)CC1] | 1-(4-((2R,3R)-1-(2- (difluoromethyl)-6-((2R,4R)- 4-(1-(1-(methoxy- d₃)cyclopropyl)-4-methyl-1H- pyrazol-5-yl)-2- methylpiperidin-1- yl)pyrimidin-4-yl)-2- methylazetidin-3-yl)piperazin- 1-yl)prop-2-en-1-one | SFC \| 400 ChiralPak IG, 2 x 25 cm 5 μm column with a mobile phase of 40% MeOH using a flowrate of 80 mL/min |
| 2-400-2 | 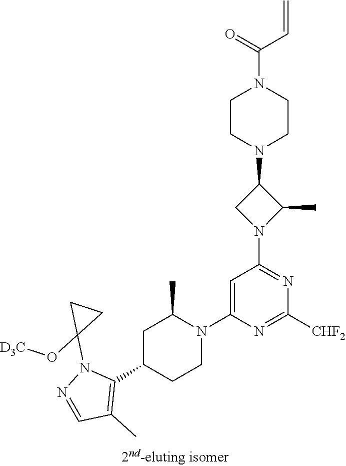 [SMILES: [2H]C([2H])([2H])OC1(n2ncc(C)c2[C@H]2CCN(c3cc(N4C[C@@H](N5CCN(C(=O)C=C)CC5)[C@H]4C)nc(C(F)F)n3)[C@H](C)C2)CC1] | 1-(4-((2R,3R)-1-(2- (difluoromethyl)-6-((2R,4S)- 4-(1-(1-(methoxy- d₃)cyclopropyl)-4-methyl-1H- pyrazol-5-yl)-2- methylpiperidin-1- yl)pyrimidin-4-yl)-2- methylazetidin-3-yl)piperazin- 1-yl)prop-2-en-1-one | SFC \| 400 ChiralPak IG, 2 x 25 cm 5 μm column with a mobile phase of 40% MeOH using a flowrate of 80 mL/min |
| 2-409-1 | 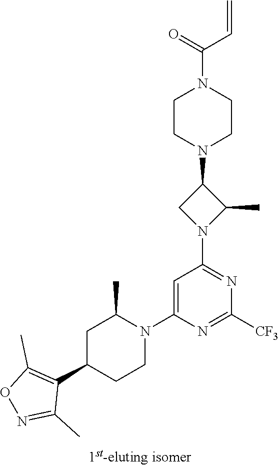 [SMILES: C=CC(=O)N1CCN([C@@H]2CN(c3cc(N4CC[C@@H](c5c(C)noc5C)C[C@H]4C)nc(C(F)(F)F)n3)[C@@H]2C)CC1] | 1-(4-((2R,3R)-1-(6-((2R.4R)- 4-(3,5-dimethyl-1,2-oxazol-4- yl)-2-methyl-1-piperidinyl)-2- (trifluoromethyl)-4- pyrimidinyl)-2-methyl-3- azetidinyl)-1-piperazinyl)-2- propon-1-one | SFC \| 409 ChiralPak AD, 2 x 25 cm 5 μm column with a mobile phase of 20% MeOH using a flowrate of 80 mL/min |
| 2-409-2 | 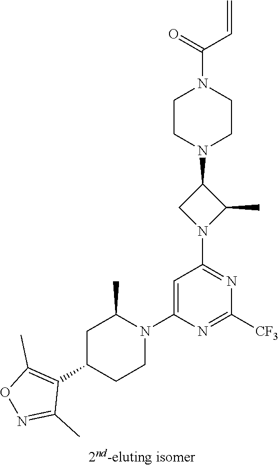 [SMILES: C=CC(=O)N1CCN([C@@H]2CN(c3cc(N4CC[C@H](c5c(C)noc5C)C[C@H]4C)nc(C(F)(F)F)n3)[C@@H]2C)CC1] | 1-(4-((2R,3R)-1-(6-((2R,4S)- 4-(3,5-dimethyl-1,2-oxazol-4- yl)-2-methyl-1-piperidinyl)-2- (trifluoromethyl)-4- pyrimidinyl)-2-methyl-3- azetidinyl)-1-piperazinyl)-2- propen-1-one | SFC \| 409 ChiralPak AD, 25 x 2 cm 5 μm column with a mobile phase of 20% MeOH using a flowrate of 80 mL/min |
| 2-428-1 | 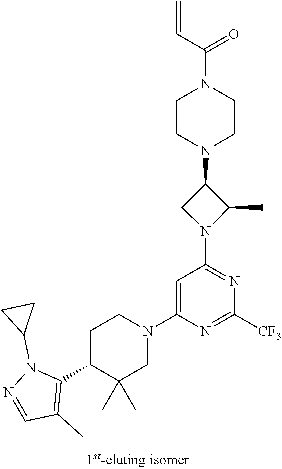 [SMILES: C=CC(=O)N1CCN([C@@H]2CN(c3cc(N4CC[C@@H](c5c(C)cnn5C5CC5)C(C)(C)C4)nc(C(F)(F)F)n3)[C@@H]2C)CC1] | 1-(4-((2R.3R)-1-(6-((4R)-4-(1- cyclopropyl-4-methyl-1H- pyrazol-5-yl)-3,3-dimethyl-1- piperidinyl)-2- (trifluoromethyl)-4- pyrimidinyl)-2-methyl-3- azetidinyl)-1-piperazinyl)-2- propen-1-one | SFC \| 428 Chiralpak IG, 250 x 30 mm, 10 μm column with a mobile phase of 50% EtOH using a flow rate of 70 mL/min |
| 2-428-2 | 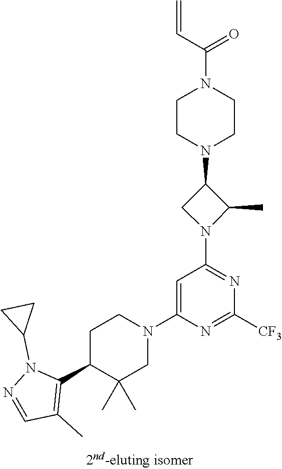 [SMILES: C=CC(=O)N1CCN([C@@H]2CN(c3cc(N4CC[C@H](c5c(C)cnn5C5CC5)C(C)(C)C4)nc(C(F)(F)F)n3)[C@@H]2C)CC1] | 1-(4-((2R,3R)-1-(6-((4S)-4-(1- cyclopropyl-4-methyl-1H- pyrazol-5-yl)-3,3-dimethyl-1- piperidinyl)-2- (trifluoromethyl)-4- pyrimidinyl)-2-methyl-3- azetidinyl)-1-piperazinyl)-2- propen-1-one | SFC \| 428 Chiralpak IG, 250 x 30 mm, 10 μm column with a mobile phase of 50% EtOH using a flow rate of 3.4 mL/min |
| 2-439-1 | 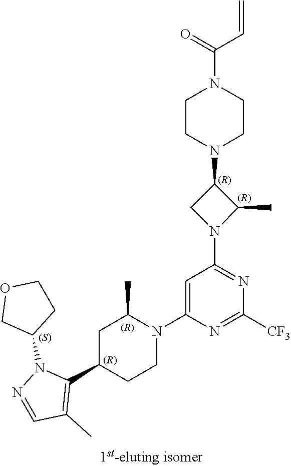 | 1-(4-((2R,3R)-2-methyl-1-(6- ((2R.4R)-2-methyl-4-(4- methyl-1-((S)-tetrahydrofuran- 3-yl)-1H-pyrazol-5- yl)piperidin-1-yl)-2- (trifluoromethyl)pyrimidin-4- yl)azetidin-3-yl)piperazin-1- yl)prop-2-en-1-one | SFC \| 439 Chiralpak IF, 30 x 250 mm 5 μm column using a mobile phase of 25% MeOH using a flow rate of 200 mL/min |
| 2-457-1 | 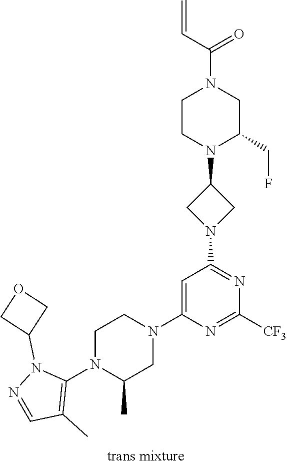 | 1-((3R)-3-(fluoromethyl)-4- (trans-3-(6-((3R)-3-methyl-4- (4-methyl-1-(3-oxetanyl)-1H- pyrazol-5-yl)-1-piperazinyl)-2- (trifluoromethyl)-4- pyrimidinyl)cyclobutyl)-1- piperazinyl)-2-propen-1-one | SFC \| 457 Chiralpak ID, 2 x 25 cm 5 μm column using a mobile phase of 40% IPA using a flow rate of 80 mL/min |
| 2-462-1 | 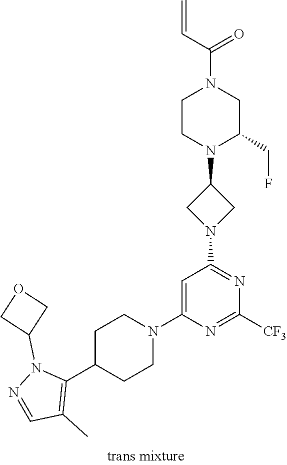 | 1-((3R)-3-(fluoromethyl)-4- (trans-3-(6-(4-(4-methyl-1-(3- oxetanyl)-1H-pyrazol-5-yl)-1- piperidinyl)-2- (trifluoromethyl)-4- pyrimidinyl)cyclobutyl)-1- piperazinyl)-2-propen-1-one | SFC \| 462 Chiralpak ID, 21 x 250 mm 5 μm column using a mobile phase of 45% IPA using a flow rate of 110 mL/min |
| 2-466-1 | 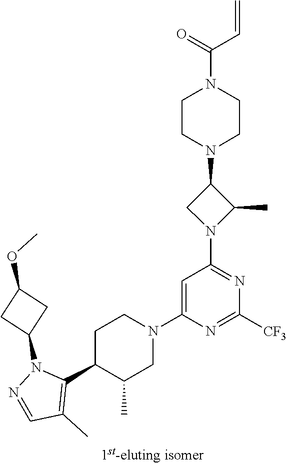 [SMILES: C=CC(=O)N1CCN([C@@H]2CN(c3cc(N4CC[C@H](c5c(C)cnn5[C@H]5C[C@@H](OC)C5)[C@@H](C)C4)nc(C(F)(F)F)n3)[C@@H]2C)CC1] | 1-(4-((2R,3R)-1-(6-((3R,4S)- 4-(1-(cis-3- methoxycyclobutyl)-4-methyl- 1H-pyrazol-5-yl)-3-methyl-1- piperidinyl)-2- (trifluoromethyl)-4- pyrimidinyl)-2-methyl-3- azetidinyl)-1-piperazinyl)-2- propen-1-one | SFC \| 466 Chiralpak ID 2 x 25 cm 5 μm column with a mobile phase of 55% IPA using a flow rate of 65 mL/min |
| 2-466-2 | 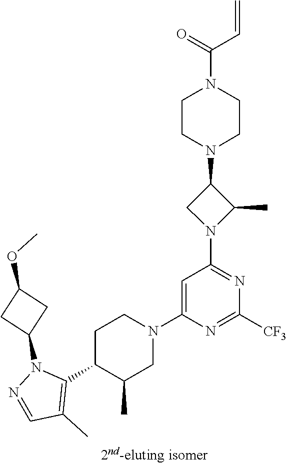 [SMILES: C=CC(=O)N1CCN([C@@H]2CN(c3cc(N4CC[C@@H](c5c(C)cnn5[C@H]5C[C@@H](OC)C5)[C@H](C)C4)nc(C(F)(F)F)n3)[C@@H]2C)CC1] | 1-(4-((2R,3R)-1-(6-((3S,4R)- 4-(1-(cis-3- methoxycyclobutyl)-4-methyl- 1H-pyrazol-5-yl)-3-methyl-1- piperidinyl)-2- (trifluoromethyl)-4- pyrimidinyl)-2-methyl-3- azetidinyl)-1-piperazinyl)-2- propen-1-one | SFC \| 466 Chiralpak ID 2 x 25 cm 5 μm column with a mobile phase of 55% IPA asing a flow rate of 65 mL/min |
| 2-489-1 | 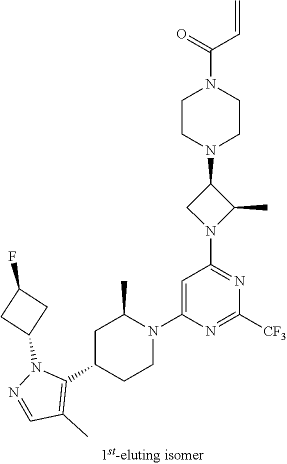 [SMILES: C=CC(=O)N1CCN([C@@H]2CN(c3cc(N4CC[C@H](c5c(C)cnn5[C@H]5C[C@H](F)C5)C[C@H]4C)nc(C(F)(F)F)n3)[C@@H]2C)CC1] | 1-(4-((2R,3R)-1-(6-((2R,4S)- 4-(1-(trans-3- fluorocyclobutyl)-4-methyl- 1H-pyrazol-5-yl)-2-methyl-1- piperidinyl)-2- (trifluoromethyl)-4- pyrimidinyl)-2-methyl-3- azetidinyl)-1-piperazinyl)-2- propen-1-one | SFC \| 489 Chiralcel OJ 21 x 250 mm 5 μm column with a mobile phase of 15% McOH using a flow rate of 150 mL/min |
| 2-490-1 | 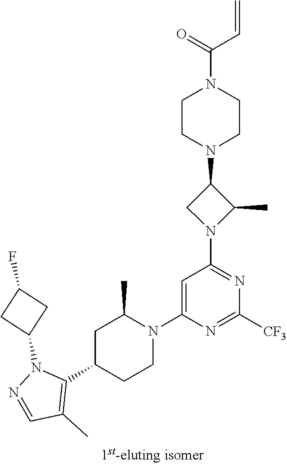 [SMILES: C=CC(=O)N1CCN([C@@H]2CN(c3cc(N4CC[C@H](c5c(C)cnn5[C@H]5C[C@@H](F)C5)C[C@H]4C)nc(C(F)(F)F)n3)[C@@H]2C)CC1] | 1-(4-((2R,3R)-1-(6-((2R,4S)- 4-(1-(cis-3-fluorocyclobutyl)- 4-methyl-1H-pyrazol-5-yl)-2- methyl-1-piperidinyl)-2- (trifluoromethyl)-4- pyrimidinyl)-2-methyl-3- azetidinyl)-1-piperazinyl)-2- propen-1-one | SFC \| 490 Chiralpak ID 21 x 250 mm 5 μm column with a mobile phase of 35% MeOH using a flow rate of 130 mL/min |
| 2-491-1 | 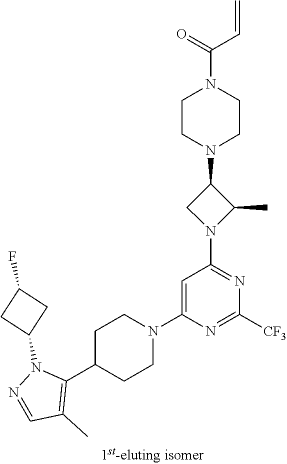 [SMILES: C=CC(=O)N1CCN([C@@H]2CN(c3cc(N4CCC(c5c(C)cnn5[C@H]5C[C@@H](F)C5)CC4)nc(C(F)(F)F)n3)[C@@H]2C)CC1] | 1-(4-((2R,3R)-1-(6-(4-(1-(cis- 3-fluorocyclobutyl)-4-methyl- 1H-pyrazol-5-yl)-1- piperidinyl)-2- (trifluoromethyl)-4- pyrimidinyl)-2-methyl-3- azetidinyl)-1-piperazinyl)-2- propen-1-one | SFC \| 491 Chiralcel OX 21 x 250 mm 5 μm column with a mobile phase of 40% MeOH using a flow rate of 100 mL/min |
| 2-491-2 | 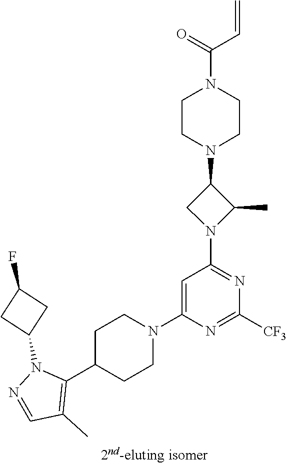 [SMILES: C=CC(=O)N1CCN([C@@H]2CN(c3cc(N4CCC(c5c(C)cnn5[C@H]5C[C@H](F)C5)CC4)nc(C(F)(F)F)n3)[C@@H]2C)CC1] | 1-(4-((2R,3R)-1-(6-(4-(1- (trans-3-fluorocyclobutyl)-4- methyl-1H-pyrazol-5-yl)-1- piperidinyl)-2- (trifluoromethyl)-4- pyrimidinyl)-2-methyl-3- azetidinyl)-1-piperazinyl)-2- propen-1-one | SFC \| 491 Chiralcel OX 21 x 250 mm 5 μm column with a mobile phase of 40% MeOH using a flow rate of 100 mL/min |
| 2-492-1 | 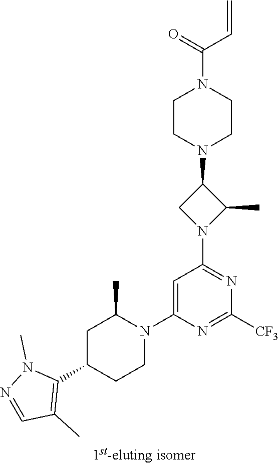 [SMILES: C=CC(=O)N1CCN([C@@H]2CN(c3cc(N4CC[C@H](c5c(C)cnn5C)C[C@H]4C)nc(C(F)(F)F)n3)[C@@H]2C)CC1] | 1-(4-((2R,3R)-1-(6-((2R,4S)- 4-(1,4-dimethyl-1H-pyrazol-5- yl)-2-methyl-1-piperidinyl)-2- (trifluoromethyl)-4- pyrimidinyl)-2-methyl-3- azetidinyl)-1-piperazinyl)-2- propen-1-one | SFC \| 492 Chiralpak AS 2 x 25 cm 5 μm column with a mobile phase of 20% MeOH using a flow rate of 80 mL/min |
| 2-493-1 | 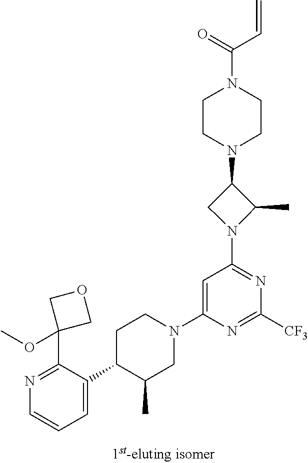 [SMILES: C=CC(=O)N1CCN([C@@H]2CN(c3cc(N4CC[C@@H](c5cccnc5C5(OC)COC5)[C@H](C)C4)nc(C(F)(F)F)n3)[C@@H]2C)CC1] | 1-(4-((2R,3R)-1-(6-((3S,4R)- 4-(2-(3-methoxy-3-oxetanyl)- 3-pyridinyl)-3-methyl-1- piperidinyl)-2- (trifluoromethyl)-4- pyrimidinyl)-2-methyl-3- azetidinyl)-1-piperazinyl)-2- propen-1-one | SFC \| 493 Lux Cellulose-2 21 x 250 mm 5 μm column with a mobile phase of 45% MeOH using a flow rate of 80 mL/min |
| 2-493-2 | 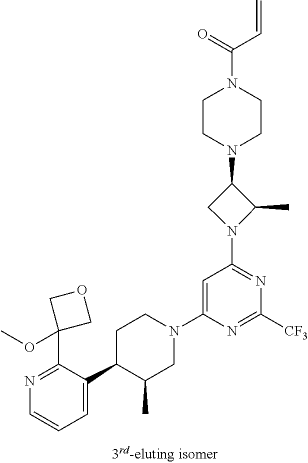 [SMILES: C=CC(=O)N1CCN([C@@H]2CN(c3cc(N4CC[C@H](c5cccnc5C5(OC)COC5)[C@H](C)C4)nc(C(F)(F)F)n3)[C@@H]2C)CC1] | 1-(4-((2R,3R)-1-(6-((3S,4S)-4- (2-(3-methoxy-3-oxetanyl)-3- pyridinyl)-3-methyl-1- piperidinyl)-2- (trifluoromethyl)-4- pyrimidinyl)-2-methyl-3- azetidinyl)-1-piperazinyl)-2- propen-1-one | SFC \| 493 Lux Cellulose-2 21 x 250 mm 5 μm column with a mobile phase of 45% MeOH using a flow rate of 80 mL/min |
| 2-493-3 | 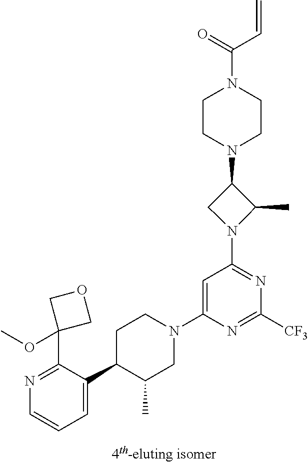 [SMILES: C=CC(=O)N1CCN([C@@H]2CN(c3cc(N4CC[C@H](c5cccnc5C5(OC)COC5)[C@@H](C)C4)nc(C(F)(F)F)n3)[C@@H]2C)CC1] | 1-(4-((2R,3R)-1-(6-((3R,4S)- 4-(2-(3-methoxy-3-oxetanyl)- 3-pyridinyl)-3-methyl-1- piperidinyl)-2- (trifluoromethyl)-4- pyrimidinyl)-2-methyl-3- azetidinyl)-1-piperazinyl)-2- propen-1-one | SFC \| 493 Lux Cellulose-2 21 x 250 mm 5 μm column with a mobile phase of 45% MeOH using a flow rate of 80 mL/min |
| 2-494-1 | 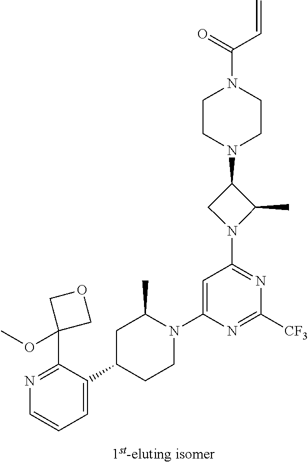 [SMILES: C=CC(=O)N1CCN([C@@H]2CN(c3cc(N4CC[C@H](c5cccnc5C5(OC)COC5)C[C@H]4C)nc(C(F)(F)F)n3)[C@@H]2C)CC1] | 1-(4-((2R,3R)-1-(6-((2R,4S)- 4-(2-(3-methoxy-3-oxctanyl)- 3-pyridinyl)-2-methyl-1- piperidinyl)-2- (trifluoromethyl)-4- pyrimidinyl)-2-methyl-3- azetidinyl)-1-piperazioyl)-2- propen-1-one | SFC \| 494 Chiralcel OD 21 x 250 mm 5 μm column with a mobile phase of 20% MeOH using a flow rate of 150 mL/min |
| 2-495-1 | 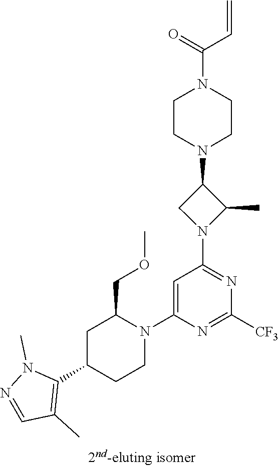 [SMILES: C=CC(=O)N1CCN([C@@H]2CN(c3cc(N4CC[C@H](c5c(C)cnn5C)C[C@H]4COC)nc(C(F)(F)F)n3)[C@@H]2C)CC1] | 1-(4-((2R,3R)-1-(6-((2S,4S)-4- (1,4-dimethyl-1H-pyrazol-5- yl)-2-(methoxymethyl)-1- piperidinyl)-2- (trifluoromethyl)-4- pyrimidinyl)-2-methyl-3- azetidinyl)-1-piperazinyl)-2- propen-1-one | SFC \| 495 Chiralcel OX, 21 x 250 mm 5 μm, column with a mobile phase of 30% (1:1) methanol:acetonitrile using a flowrate of 125 mL/min |
| 2-496-1 | 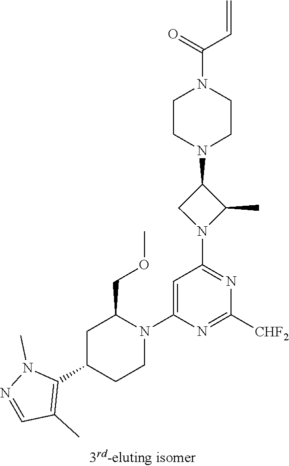 [SMILES: C=CC(=O)N1CCN([C@@H]2CN(c3cc(N4CC[C@H](c5c(C)cnn5C)C[C@H]4COC)nc(C(F)F)n3)[C@@H]2C)CC1] | 1-(4-((2R,3R)-1-(2- (difluoromethyl)-6-((2S,4S)-4- (1,4-dimethyl-1H-pyrazol-5- yl)-2-(methoxymethyl)-1- piperidinyl)-4-pyrimidinyl)-2- methyl-3-azetidinyl)-1- piperazinyl)-2-propen-1-one | SFC \| 496 Chiralcel OX, 21 x 250 mm 5 μm, column with a mobile phase of 45% 2- propanol with 0.2% tricthylamine using a flowrate of 80 mL/min |
| 2-507-1 | 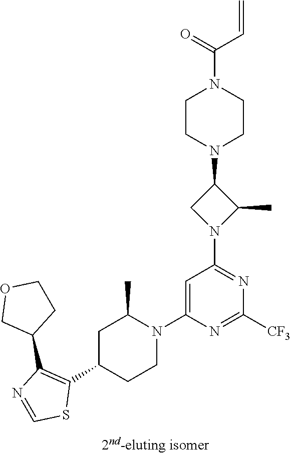 [SMILES: C=CC(=O)N1CCN([C@@H]2CN(c3cc(N4CC[C@H](c5scnc5[C@@H]5CCOC5)C[C@H]4C)nc(C(F)(F)F)n3)[C@@H]2C)CC1] | 1-(4-((2R,3R)-2-methyl-1-(6- ((2R,4S)-2-methyl-4-(4-((3S)- tetrahydro-3-furanyl)-1,3- thiazol-5-yl)-1-piperidinyl)-2- (trifluoromethyl)-4- pyrimidinyl)-3-azetidinyl)-1- piperazinyl)-2-propen-1-one | SFC \| 507 Chiralpak AS 2 x 25 cm 5 μm column with a mobile phase of 20% MeOH using a flow rate of 80 mL/min |
| 2-507-2 | 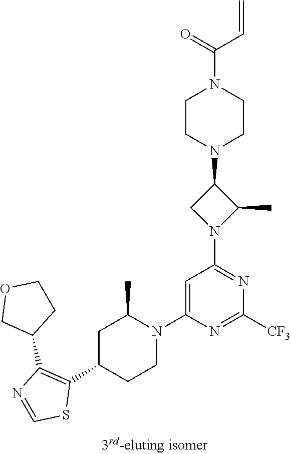 [SMILES: C=CC(=O)N1CCN([C@@H]2CN(c3cc(N4CC[C@H](c5scnc5[C@H]5CCOC5)C[C@H]4C)nc(C(F)(F)F)n3)[C@@H]2C)CC1] | 1-(4-((2R,3R)-2-methyl-1-(6- ((2R,4S)-2-methyl-4-(4-((3R)- tetrahydro-3-furanyl)-1,3- thiazol-5-yl)-1-piperidinyl)-2- (trifluoromethyl)-4- pyrimidinyl)-3-azetidinyl)-1- piperazinyl)-2-propen-1-one | SFC \| 507 Chiralpak AS 2 x 25 cm 5 μm column with a mobile phase of 20% MeOH using a flow rate of 80 mL/min |
| 2-510-1 | 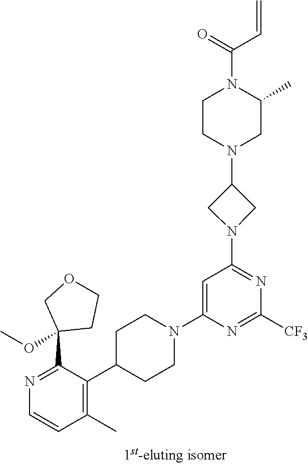 [SMILES: C=CC(=O)N1CCN(C2CN(c3cc(N4CCC(c5c(C)ccnc5[C@]5(OC)CCOC5)CC4)nc(C(F)(F)F)n3)C2)C[C@H]1C] | 1-((2R)-4-(1-(6-(4-(2-((3R)-3- methoxytetrahydro-3-furanyl)- 4-methyl-3-pyridinyl)-1- piperidinyl)-2- (trifluoromethyl)-4- pyrimidinyl)-3-azetidinyl)-2- methyl-1-piperazinyl)-2- propen-1-one | SFC \| 510 Chiralpak AD 2 x 25 cm 5 μm column with a mobile phase of 35% IPA using a flow rate of 80 mL/min |
| 2-510-2 | 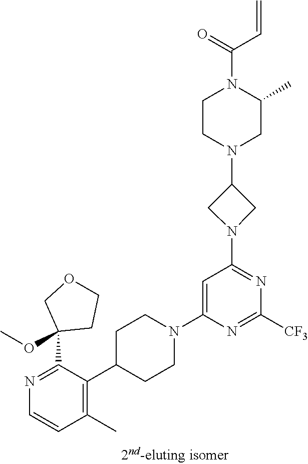 [SMILES: C=CC(=O)N1CCN(C2CN(c3cc(N4CCC(c5c(C)ccnc5[C@@]5(OC)CCOC5)CC4)nc(C(F)(F)F)n3)C2)C[C@H]1C] | 1-((2R)-4-(1-(6-(4-(2-((3S)-3- methoxytetrahydro-3-furanyl)- 4-methyl-3-pyridinyl)-1- pipcridinyl)-2- (trifluoromethyl)-4- pyrimidinyl)-3-azetidinyl)-2- methyl-1-piperazinyl)-2- propen-1-one | SFC \| 510 Chiralpak AD 2 x 25 cm 5 μm column with a mobile phase of 35% IPA using a flow rate of 80 mL/min |
| 2-516-1 | 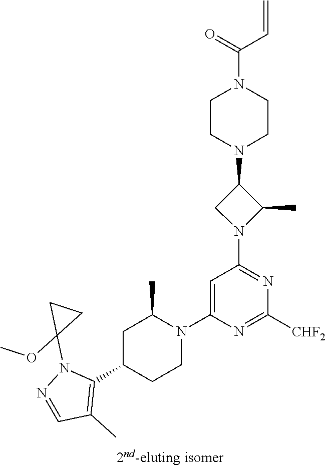 [SMILES: C=CC(=O)N1CCN([C@@H]2CN(c3cc(N4CC[C@H](c5c(C)cnn5C5(OC)CC5)C[C@H]4C)nc(C(F)F)n3)[C@@H]2C)CC1] | 1-(4-((2R,3R)-1-(2- (difluoromethyl)-6-((2R,4S)- 4-(1-(1-methoxycyclopropyl)- 4-methyl-1H-pyrazol-5-yl)-2- methyl-1-piperidinyl)-4- pyrimidinyl)-2-methyl-3- azetidinyl)-1-piperazinyl)-2- propen-1-one | Chiralpal IG 21 x 150 mm 5 μm, column with a mobile phase of 40% MeOH using a flow rate of 150 mL/min |
| 2-520-1 | 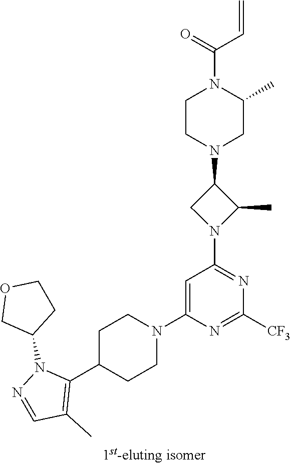 [SMILES: C=CC(=O)N1CCN([C@@H]2CN(c3cc(N4CCC(c5c(C)cnn5[C@H]5CCOC5)CC4)nc(C(F)(F)F)n3)[C@@H]2C)C[C@H]1C] | 1-((2R)-2-methyl-4-((2R,3R)- 2-methyl-1-(6-(4-(4-methyl-1- ((3S)-tetrahydro-3-furanyl)- 1H-pyrazol-5-yl)-1- piperidinyl)~2- (trifluoromethyl)-4- pyrimidinyl)-3-azetidinyl)-1- piperazinyl)-2-propen-1-one | Chiralpal OD 21 x 250 mm 5 μm, column with a mobile phase of 20% IPA using a flow rate of 100 mL/min |
| 2-520-2 | 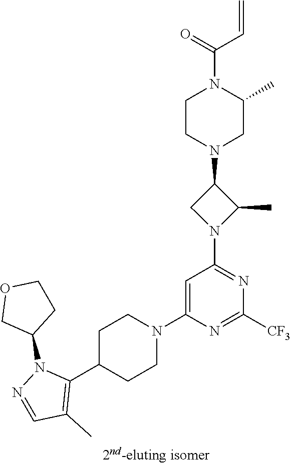 [SMILES: C=CC(=O)N1CCN([C@@H]2CN(c3cc(N4CCC(c5c(C)cnn5[C@@H]5CCOC5)CC4)nc(C(F)(F)F)n3)[C@@H]2C)C[C@H]1C] | 1-((2R)-2-methyl-4-((2R,3R)- 2-methyl-1-(6-(4-(4-methyl-1- ((3R)-tetrahydro-3-furanyl)- 1H-pyrazol-5-yl)-1- piperidinyl)-2- (trifluoromethyl)-4- pyrimidinyl)-3-azetidinyl)-1- piperazinyl)-2-propen-1-one | Chiralpal OD 21 x 250 mm 5 μm, column with a mobile phase of 20% IPA asing a flow rate of 100 mL/min |
| 2-531-1 | 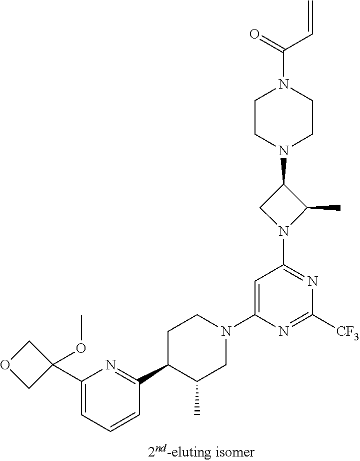 [SMILES: C=CC(=O)N1CCN([C@@H]2CN(c3cc(N4CC[C@H](c5cccc(C6(OC)COC6)n5)[C@@H](C)C4)nc(C(F)(F)F)n3)[C@@H]2C)CC1] | 1-(4-((2R,3R)-1-(6-((3R,4S)- 4-(6-(3-methoxy-3-oxctanyl)- 2-pyridinyl)-3-methyl-1- piperidinyl)-2- (trifluoromethyl)-4- pyrimidinyl)-2-methyl-3- azetidinyl)-1-piperazinyl)-2- propen-1-one | SFC \| 531 Chiralcel OD, 2 x 25 cm 5 μm column with a mobile phase of 15% MeOH using a flowrate of 80 mL/min |
| 2-532-1 | 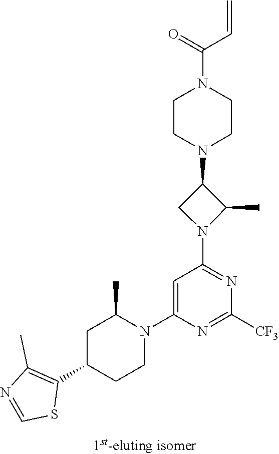 [SMILES: C=CC(=O)N1CCN([C@@H]2CN(c3cc(N4CC[C@H](c5scnc5C)C[C@H]4C)nc(C(F)(F)F)n3)[C@@H]2C)CC1] | 1-(4-((2R,3R)-2-methyl-1-(6- ((2R.4S)-2-methyl-4-(4- methyl-1,3-thiazol-5-yl)-1- piperidinyl)-2- (trifluoromethyl)-4- pyrimidinyl)-3-azetidinyl)-1- piperazinyl)-2-propen-1-one | Chiralcel OD, 2 x 25 cm 5 μm column with a mobile phase of 35% MeOH using a flowrate of 80 mL/min |
| 2-533-1 | 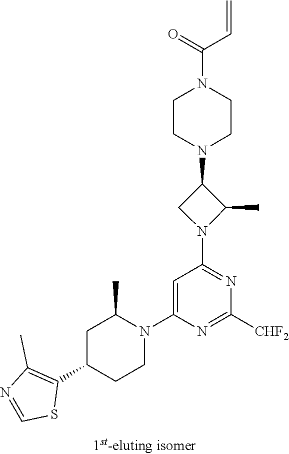 [SMILES: C=CC(=O)N1CCN([C@@H]2CN(c3cc(N4CC[C@H](c5scnc5C)C[C@H]4C)nc(C(F)F)n3)[C@@H]2C)CC1] | 1-(4-((2R,3R)-1-(2- (difluoromethyl)-6-((2R,4S)- 2-methyl-4-(4-methyl-1,3- thiazol-5-yl)-1-piperidinyl)-4- pyrimidinyl)-2-methyl-3- azetidinyl)-1-piperazinyl)-2- propen-1-one | Chiralcel OD, 2 x 25 cm 5 μm column with a mobile phase of 40% MeOH using a flowrate of 80 mL/min |
| 2-534-1 | 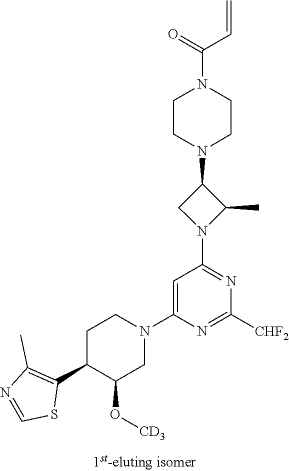 [SMILES: [2H]C([2H])([2H])O[C@@H]1CN(c2cc(N3C[C@@H](N4CCN(C(=O)C=C)CC4)[C@H]3C)nc(C(F)F)n2)CC[C@@H]1c1scnc1C] | 1-(4-((2R,3R)-1-(6-((3R,4R)- 3-(methoxy-d3)-4-(4- methylthiazol-5-yl)piperidin- 1-yl)-2- (trifluoromethyl)pyrimidin-4- yl)-2-methylazetidin-3- yl)piperazin-1-yl)prop-2-en-1- one | Chiralpak IG, 21 x 250 mm 5 μm, column with a mobile phase of 40% 2-propanol using a flowrate of 80 mL/min |

The stereochemistry of the structures was arbitrarily assigned and is not established.

### Example 2-103: (R)-1-(2-methyl-4-(1-(6-(4-(4-methyl-1-(oxetan-3-yl)-1H-pyrazol-5-yl)piperidin-1-yl)-2-(trifluoromethyl)pyrimidin-4-yl)azetidin-3-yl)piperazin-1-yl)prop-2-en-1-one-2,3,3-d3

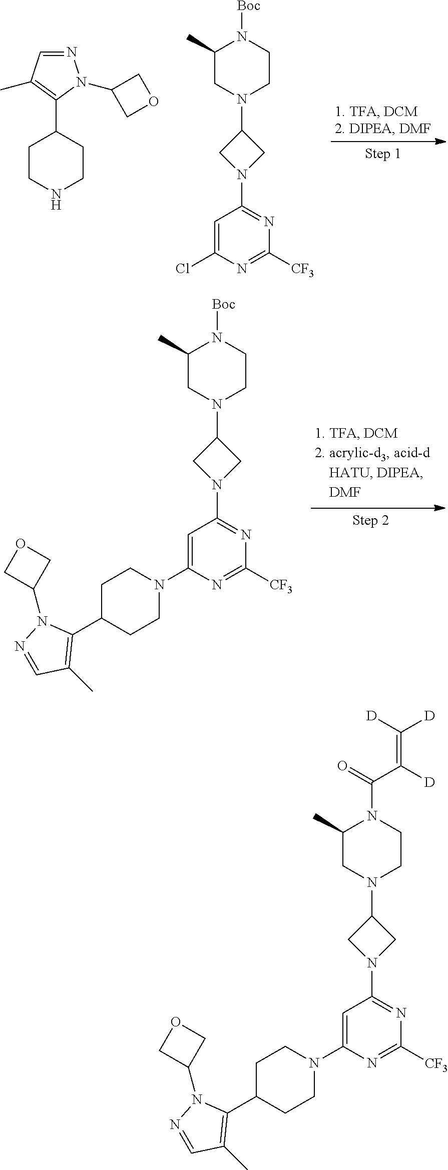

Step 1. A mixture of tert-butyl (R)-4-(1-(6-chloro-2-(trifluoromethyl)pyrimidin-4-yl)azetidin-3-yl)-2-methylpiperazine-1-carboxylate (214 mg, 0.491 mmol). DIPEA (0.26 mL, 1.47 mmol, Sigma-Aldrich. Inc.), and 4-(4-methyl-1-(oxetan-3-yl)-1H-pyrazol-5-yl)piperidine (293 mg, 1.33 mmol in DMF (2.3 mL) was stirred at 95° C. for 18 h. The mixture was then diluted with sat. aq. Na₂CO₃ (30 mL) and extracted with EtOAc (2×30 mL). The combined organic extracts were dried over MgSO₄ and concentrated in vacuo. Chromatographic purification of the residue (silica gel, (0%-100% EtOAc:EtOH (3:1)/heptane) provided tert-butyl (R)-2-methyl-4-(1-(6-(4-(4-methyl-1-(oxetan-3-yl)-1H-pyrazol-5-yl)piperidin-1-yl)-2-(trifluoromethyl)pyrimidin-4-yl)azetidin-3-yl)piperazine-1-carboxylate. m/z (ESI): 621.1 (M+H)⁺.

Step 2. To a solution of tert-butyl (R)-2-methyl-4-(1-(6-(4-(4-methyl-1-(oxetan-3-yl)-1H-pyrazol-5-yl)piperidin-1-yl)-2-(trifluoromethyl)pyrimidin-4-yl)azetidin-3-yl)piperazine-1-carboxylate (305 mg, 0.491 mmol) in DCM (2 mL) was added TFA (0.7 mL, 9.83 mmol), and the reaction mixture was stirred at rt for 1 h. The mixture was then poured into an ice water containing saturated K₂CO₃ (50 mL) and extracted with EtOAc (3×50 mL). The combined organic extracts were then dried over MgSO₄, concentrated, and dried in vacuo to provide (R)-4-(4-(4-methyl-1-(oxetan-3-yl)-1H-pyrazol-5-yl)piperidin-1-yl)-6-(3-(3-methylpiperazin-1-yl)azetidin-1-yl)-2-(trifluoromethyl)pyrimidine (256 mg, 0.492 mmol, quantitative). A mixture of (R)-4-(4-(4-methyl-1-(oxetan-3-yl)-1H-pyrazol-5-yl)piperidin-1-yl)-6-(3-(3-methylpiperazin-1-yl)azetidin-1-yl)-2-(trifluoromethyl)pyrimidine (48 mg, 0.092 mmol), acrylic acid-d₄ (14 mg, 0.184 mmol, Aablocks, Inc.). DIPEA (0.048 mL, 0.277 mmol, Sigma-Aldrich. Inc.), and HATU (35 mg, 0.092 mmol, Combi-Blocks, Inc.) in DMF (1 mL) was stirred at rt for 18 h. The mixture was diluted with sat. aq. Na₂CO₃ (10 mL) and extracted with EtOAc (2×20 mL). The combined organic extracts were dried over MgSO₄ and concentrated in vacuo. Chromatographic purification of the residue (silica gel, 0% to 100% (3:1) EtOAc:EtOH in heptane) provided (R)-1-(2-methyl-4-(1-(6-(4-(4-methyl-1-(oxetan-3-yl)-1H-pyrazol-5-yl)piperidin-1-yl)-2-(trifluoromethyl)pyrimidin-4-yl)azetidin-3-yl)piperazin-1-yl)prop-2-en-1-one-2,3,3-d3. m/z (ESI): 578.2 (M+H)⁺. ¹H NMR (500 MHz, DMSO-d₆) δ ppm 7.29 (s, 1H), 5.64-5.73 (m, 2H), 4.90-4.94 (m, 2H), 4.84-4.88 (m, 2H), 4.45 (br d, J=12.6 Hz, 2H), 4.16-4.32 (m, 1H), 4.05 (br t, J=7.7 Hz, 2H), 3.85 (dd, J=9.0, 4.9 Hz, 1H), 3.78 (br dd, J=9.0, 5.0 Hz, 1H), 3.35-3.42 (m, 1H), 3.20-3.28 (m, 2H), 2.99-3.07 (m, 1H), 2.89-2.96 (m, 2H), 2.80-2.87 (m, 1H), 2.69 (br d, J=10.9 Hz, 1H), 1.98 (s, 4H), 1.80-1.91 (m, 1H), 1.63-1.78 (m, 4H), 1.10-1.31 (m, 3H).

The example in the table below was prepared in a manner similar to that described above using the indicated pyrimidinyl halide.

| | | | Pyrimidinyl | m/z (ESI): |
|---|---|---|---|---|
| Ex. | Chemical Structure | Name | halide | (M + H)⁺ |
| 2-471 | 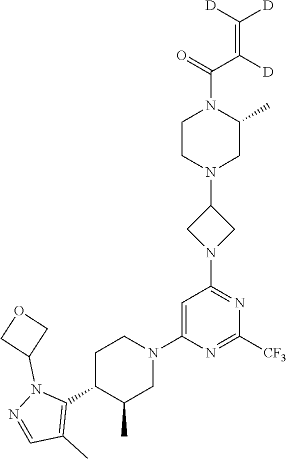 [SMILES: [2H]C([2H])=C([2H])C(=O)N1CCN(C2CN(c3cc(N4CC[C@@H](c5c(C)cnn5C5COC5)[C@H](C)C4)nc(C(F)(F)F)n3)C2)C[C@H]1C] | 1-((R)-2-methyl-4-(1-(6- ((3S,4R)-3-methyl-4-(4- methyl-1-(oxetan-3-yl])- 1H-pyrazol-5-yl)piperidin- 1-yl)-2-(trifluoromethyl)- pyrimidin-4-yl)azetidin- 3-yl)piperazin-1-yl)prop- 2-en-1-one-2,3,3-d₃ | E-4 | 592.0 |

### Example 2-104: 1-(4-((2R,3R)-2-methyl-1-(6-((1S,5S,6S)-5-(4-methyl-1-(oxetan-3-yl)-1H-pyrazol-5-yl)-2-azabicyclo[4.1.0]heptan-2-yl)-2-(trifluoromethyl)pyrimidin-4-yl)azetidin-3-yl)piperazin-1-yl)prop-2-en-1-one

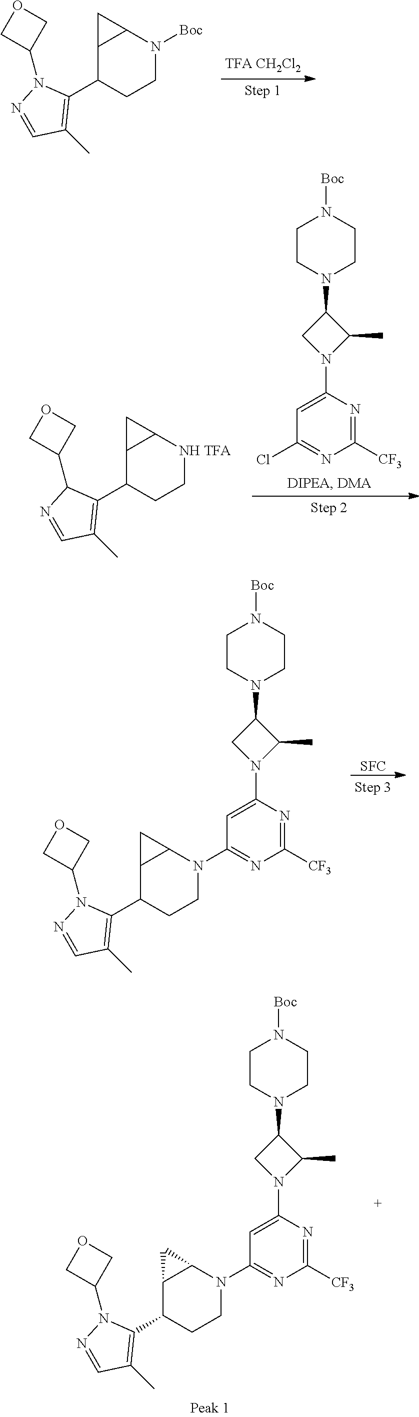

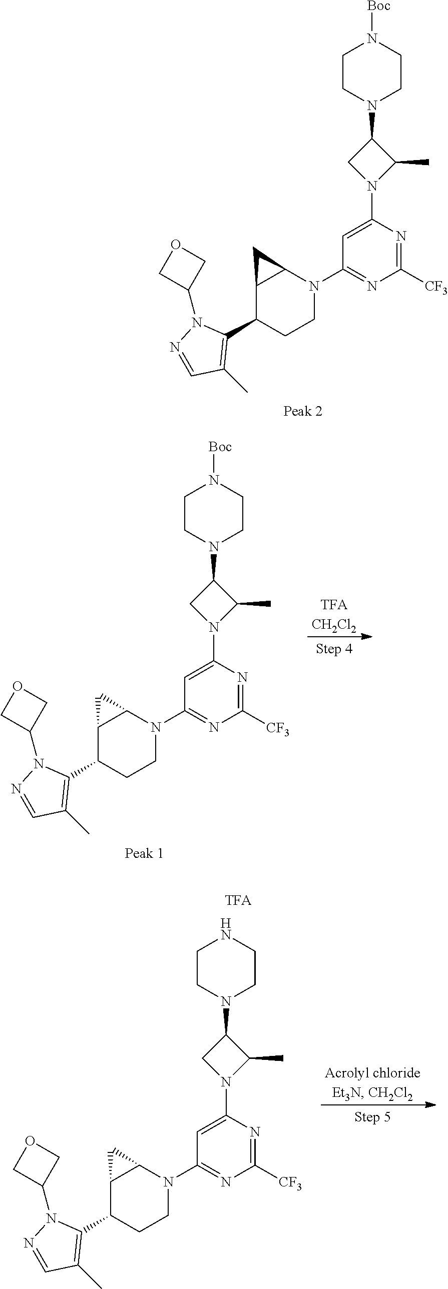 [SMILES: "TFACH2Cl2" ; Cc1cnn(C2COC2)c1[C@@H]1CCN(c2cc(N3C[C@@H](N4CCN(C(=O)OC(C)(C)C)CC4)[C@H]3C)nc(C(F)(F)F)n2)[C@@H]2C[C@@H]21 ; Cc1cnn(C2COC2)c1[C@H]1CCN(c2cc(N3C[C@@H](N4CCN(C(=O)OC(C)(C)C)CC4)[C@H]3C)nc(C(F)(F)F)n2)[C@H]2C[C@@H]12 ; Cc1cnn(C2COC2)c1[C@H]1CCN(c2cc(N3C[C@@H](N4CCNCC4)[C@H]3C)nc(C(F)(F)F)n2)[C@H]2C[C@@H]12 ; "Acrolyl chlorideEt3N, CH2Cl2"]

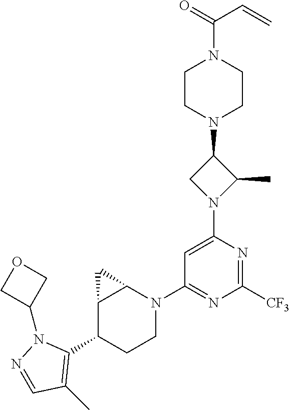 [SMILES: C=CC(=O)N1CCN([C@@H]2CN(c3cc(N4CC[C@H](c5c(C)cnn5C5COC5)[C@@H]5C[C@@H]54)nc(C(F)(F)F)n3)[C@@H]2C)CC1]

Step 1. To a solution of tert-butyl (1S,6S)-5-(4-methyl-1-(oxetan-3-yl)-1H-pyrazol-5-yl)-2-azabicyclo[4.1.0]heptane-2-carboxylate (120 mg, 0.360 mmol) in DCM (2 mL) was added TFA (83 μL, 1.08 mmol, Spectrochem) and the reaction mixture was stirred for 1 h ac rt. The reaction mixture was concentrated under reduced pressure to give 5-(4-methyl-1-(oxetan-3-yl)-1H-pyrazol-5-yl)-2-azabicyclo[4.1.0]heptane trifluoroacetate salt. m/z (ESI): 234.1 (M+H)⁺.

Step 2. Toa solution of 5(4-methyl-1-(oxetan-3-yl)-1H-pyrazol-5-yl)-2-azabicyclo[4.1.0]heptane trifluoroacetate salt (0.118 g, 0.358 mmol) and tert-butyl 4-((2R,3R)-1-(6-chloro-2-(trifluoromethylpyrimidin-4-b)-2-methyl)azetidin-3-yl)piperazine-1-carboxylate (0.13 g, 0.298 mmol) in DMA (2 mL), DIPEA (0.260 mL, 1.491 mmol, Sonia industries) was added over 5 mm at rt. The reaction mixture was stirred at 85° C. for 16 h. The reaction mixture was quenched with H₂O (10 mL), extracted with EtOAc (2×10 mL), and the organic extracts were dried over anhydrous Na₂SO₄. The solution was filtered and concentrated under reduced pressure. The crude residue was purified by chromatography (silica, 0-60% EtOAc:hexanes) to give tert-butyl 4-(2R,3R)-2-methyl-1-(6-((1S,6S)-5-(4-methyl-1-(oxetan-3-yl)-1H-pyrazol-5-yl)-2-azabicyclo[4.1.0]heptan-2-yl)-2-(trifluoromethyl)pyrimidin-4-yl)azetidin-3-yl)piperazine-1-carboxylate. m/z (ESI): 632.7 (M+H)⁺.

Step 3. The racemic mixture of tert-butyl 4-((2R,3R)-2-methyl-1-(6-((1S,6s)-5-(4-methyl-1-(oxetan-3-yl)-1H-pyrazol-5-yl)-2-azabicyclo[4.1.0]heptan-2-yl)-2-(trifluoromethyl)pyrimidin-4-yl)azetidin-3-yl)piperazine-1-carboxylate was dissolved in MeOH and chiral separation was performed in an SFC-BT-200 instrument on a Chiralpak OJ-H (250×30) mm, 5 μm column with CO₂:MeOH (80:20) at a flow rate of 80 mL/min. The configuration was assigned arbitrarily. Peak 1: 30 mg, m/z (ESI): 633.2 (M+H)⁺. Peak 2: 45 mg, m/z (ESI): 633.3 (M+H)⁺.

Step 4. To a solution of tert-butyl 4-((2R,3R)-2-methyl-1-(6-((1S,5S,6S)-5-(4-methyl-1-(oxetan-3-yl)-1H-pyrazol-5-yl)-2-azabicyclo[4.1.0]heptan-2-yl)-2-(trifluoromethyl)pyrimidin-4-yl)azetidin-3-yl)piperazine-1-carboxylate (peak 1) (30 mg, 0.047 mmol) in DCM (2.0 mL) was added TFA (11 μL, 0.142 mmol, Spectrochem) and the reaction mixture was stirred for 1 h at rt. The reaction mixture was concentrated under reduced pressure to give (1S,5S,6S)-5-(4-methyl-1-(oxetan-3-yl)-1H-pyrazol-5-yl)-2-(6-((2R,3R)-2-methyl-3-(piperazin-1-yl)azetidin-1-yl)-2-(trifluoromethyl)pyrimidin-4-yl)-2-azabicyclo[4.1.0]heptane trifluoroacetate salt. m/z (ESI): 533.3 (M+H)⁺. To a stirred solution of (1S,5S,6S)-5-(4-methyl-1-(oxetan-3-yl)-1H-pyrazol-5-yl)-2-(6-((2R,3R)-2-methyl-3-(piperazin-1-yl)azetidin-1-yl)-2-trifluoromethyl)pyrimidin-4-yl)-2-azabicyclo[4.1.0]heptane trifluoroacetate salt (29 mg, 0.046 mmol) in DCM (2.0 mL) was added TEA (19 μL, 0.138 mmol, Spectrochem) followed by acryloyl chloride (6.25 mg, 0.069 mmol, Symax Laboratories) at 0° C. The reaction mixture was stirred for 15 min at rt, quenched with ice cold water (10 mL), and extracted with DCM (2×10 mL). The combined organic extracts were washed with brine (10 mL), dried over anhydrous Na₂SO₄. and concentrated under reduced pressure. The crude material was purified by reverse phase preparative HPLC (YMC C18 (250×20) mm 5.0 μm column with a mobile phase of 0.1% NH₃ in H₂O and CH₃CN using a flow rate of 15 mL/min) followed by lyophilization to give 1-(4-((2R,3R)-2-methyl-1-(6-((1S,5S,6S)-5-(4-methyl-1-(oxetan-3-yl)-1H-pyrazol-5-yl)-2-azabicyclo[4.1.0]heptan-2-yl)-2-(trifluoromethyl)pyrimidin-4-yl)azetidin-3-yl)piperazin-1-yl)prop-2-en-1-one. m/z (ESI): 586.7 (M+H)⁺. ¹H NMR (400 MHz, DMSO-d₆) δ ppm 7.33 (s, 1H), 6.80 (dd, 1H, J=16.7, 10.5 Hz), 6.11 (dd, 1H, J=16.7, 2.4 Hz), 5.7-5.8 (m, 1H), 5.69 (dd, 1H, J=10.5, 2.4 Hz), 5.56 (s, 1H), 4.8-5.0 (m, 4H), 4.4-4.7 (m, 2H), 3.8-4.0 (m, 2H), 3.5-3.7 (s, 5H), 3.27 (d, 2H, J=7.1 Hz), 2.79 (d, 2H, J=12.9 Hz), 2.2-2.4 (m, 4H), 2.04 (s, 3H), 1.66 (d, 2H, J=16.6 Hz), 1.47 (d, 1H, J=9.1 Hz), 1.41 (d, 2H, J=6.3 Hz), 1.03 (s, 1H), 0.63 (s, 1H).

Examples in the table below were prepared in a manner similar to that described above using the indicated pyrimidinyl halide. The solvent for Step 2 was either DMF or DMA.

| | | | Pyrimidinyl | | m/z (ESI): |
|---|---|---|---|---|---|
| Ex. | Chemical Structure | Name | halide | Int.# | (M + H)⁺ |
| 2-105 | 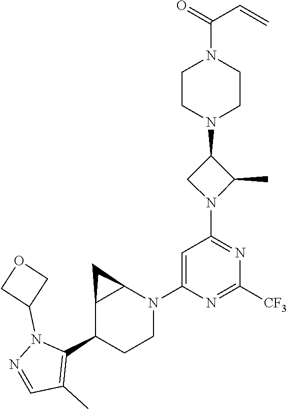 [SMILES: C=CC(=O)N1CCN([C@@H]2CN(c3cc(N4CC[C@@H](c5c(C)cnn5C5COC5)[C@H]5C[C@H]54)nc(C(F)(F)F)n3)[C@@H]2C)CC1] | 1-(4-((2R,3R)-2-methyl- 1-(6-((1R,5R,6R)-5-(4- methyl-1-(oxetan-3-yl)- 1H-pyrazol-5-yl)-2- azabicyclo[4.1.0]heptan- 2-yl)-2-trifluoromethyl)- pyrimidin-4-yl)azetidin- 3-yl)piperazin-1-yl)prop- 2-en-1-one | E-1 | H-1-2 | 586.7 |
| 2-106 | 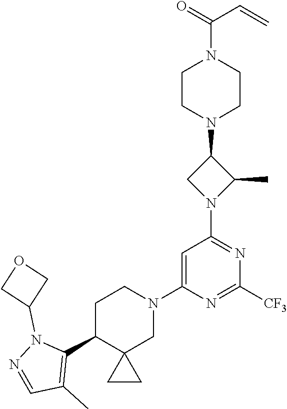 [SMILES: C=CC(=O)N1CCN([C@@H]2CN(c3cc(N4CC[C@H](c5c(C)cnn5C5COC5)C5(CC5)C4)nc(C(F)(F)F)n3)[C@@H]2C)CC1] | 1-(4-((2R,3R)-2-methyl- 1-(6-((S)-8-(4-methyl-1- (oxetan-3-yl)-1H-pyr- azol-5-yl)-5-azaspiro [2.5]octan-5-yl)-2- (trifluoromethyl)pyr- imidin-4-yl)azetidin-3- yl)piperazin-1-yl)prop- 2-en-1-one | E-8 | H-2-2 | 601.2 |
| 2-107 | 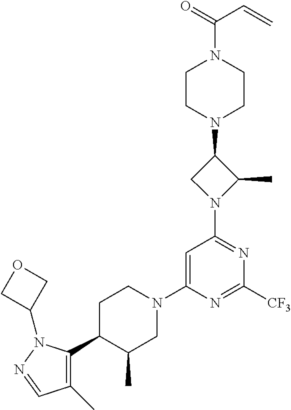 [SMILES: C=CC(=O)N1CCN([C@@H]2CN(c3cc(N4CC[C@H](c5c(C)cnn5C5COC5)[C@H](C)C4)nc(C(F)(F)F)n3)[C@@H]2C)CC1] | 1-(4-((2R,3R)-2-methyl- 1-(6-((35,4S)-3-methyl- 4-(4-methyl-1-(oxetan- 3-yl)-1H-pyrazol-5- yl)piperidin-1-yl)-2- (trifluoromethyl)- pyrimidin-4-yl)azeti- din-3-yl)piperazin-1- yl)prop-2-en-1-one | E-1 | H-3-2 | 588.8 |
| 2-108 | 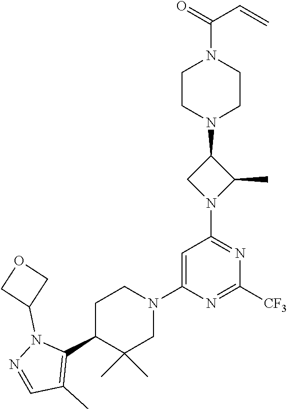 [SMILES: C=CC(=O)N1CCN([C@@H]2CN(c3cc(N4CC[C@H](c5c(C)cnn5C5COC5)C(C)(C)C4)nc(C(F)(F)F)n3)[C@@H]2C)CC1] | 1-(4-((2R,3R)-1-(6-((R)- 3,3-dimethyl-4-(4-meth- yl-1-(oxetan-3-yl)-1H- pyrazol-5-yl)piperidin- 1-yl)-2-(trifluoromethyl)- pyrimidin-4-yl)-2-meth- ylazetidin-3-yl)piper- azin-1-yl)prop-2-en-1- one | E-1 | H-4-1 | 602.7 |
| 2-109 | 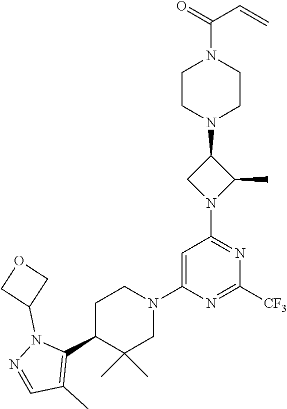 [SMILES: C=CC(=O)N1CCN([C@@H]2CN(c3cc(N4CC[C@H](c5c(C)cnn5C5COC5)C(C)(C)C4)nc(C(F)(F)F)n3)[C@@H]2C)CC1] | 1-(4-((2R,3R)-1-(6-((S)- 3,3-dimethyl-4-(4-meth- yl-1-(oxetan-3-yl)-1H- pyrazol-5-yl)piperidin- 1-yl)-2-(trifluorometh- yl)pyrimidin-4-yl)-2- methylazetidin-3-yl)- piperazin-1-yl)prop-2- en-1-one | E-1 | H-4-2 | 602.7 |
| 2-110 | 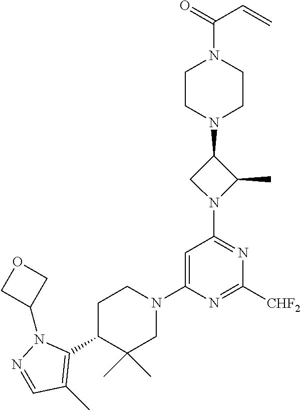 [SMILES: C=CC(=O)N1CCN([C@@H]2CN(c3cc(N4CC[C@@H](c5c(C)cnn5C5COC5)C(C)(C)C4)nc(C(F)F)n3)[C@@H]2C)CC1] | 1-(4-((2R,3R)-1-(2- (difluoromethyl)-6-((R)- 3,3-dimethyl-4-(4-meth- yl-1-(oxetan-3-yl)-1H- pyrazol-5-yl)piperidin- 1-yl)pyrimidin-4-yl)-2- methylazetidin-3-yl)- piperazin-1-yl)prop-2- en-1-one | E-2 | H-5-1 | 585.3 |
| 2-111 | 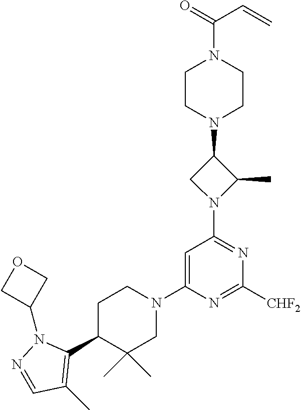 [SMILES: C=CC(=O)N1CCN([C@@H]2CN(c3cc(N4CC[C@H](c5c(C)cnn5C5COC5)C(C)(C)C4)nc(C(F)F)n3)[C@@H]2C)CC1] | 1-(4-((2R,3R)-1-(2- (difluoromethyl)-6-((S)- 3,3-dimethyl-4-(4-meth- yl-1-(oxetan-3-y1)-1H- pyrazol-5-yl)piperidin- 1-yl)pyrimidin-4-yl)-2- methylazetidin-3-yl)- piperazin-1-yl)prop-2- en-1-one | E-2 | H-5-2 | 585.3 |
| 2-112 | 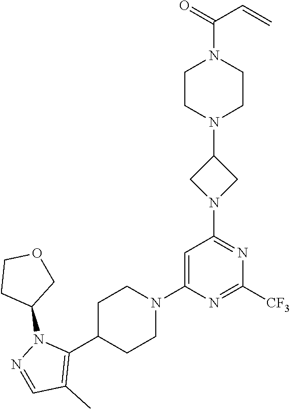 [SMILES: C=CC(=O)N1CCN(C2CN(c3cc(N4CCC(c5c(C)cnn5[C@H]5CCOC5)CC4)nc(C(F)(F)F)n3)C2)CC1] | (S)-1-(4-(1-(6-(4-(4- methyl-1-(tetrahydro- furan-3-yl)-1H-pyrazol- 5-yl)piperidin-1-yl)-2- (trifluoromethyl)pyr- imidin-4-yl)azetidin-3- yl)piperazin-1-yl)prop- 2-en-1-one | E-3 | H-6-1 | 575.2 |
| 2-113 | 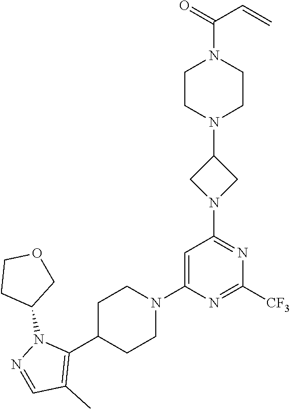 [SMILES: C=CC(=O)N1CCN(C2CN(c3cc(N4CCC(c5c(C)cnn5[C@@H]5CCOC5)CC4)nc(C(F)(F)F)n3)C2)CC1] | ((R)-1-(4-(1-(6-(4-(4- methyl-1-(tetrahydro- furan-3-yl)-1H-pyrazol- 5-yl)piperidin-1-yl)-2- (trifluoromethyl)pyr- imidin-4-yl)azetidin-3- yl)piperazin-1-yl)prop- 2-en-1-one | E-3 | H-6-2 | 575.2 |
| 2-114 | 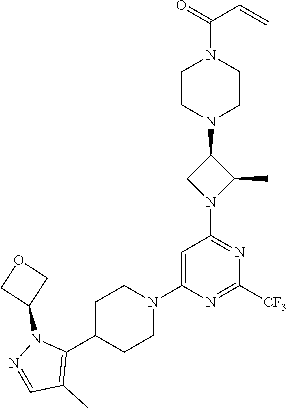 | 1-(4-((2R,3R)-2-methyl- 1-(6-(4-(4-methyl-1-((S)- tetrahydrofuran-3-yl)-1H- pyrazol-5-yl)piperidin-1- yl)-2-(trifluoromethyl)- pyrimidin-4-yl)azetidin- 3-yl)piperazin-1-yl)prop- 2-en-1-one | E-1 | H-7-1 | 589.3 |
| 2-115 | 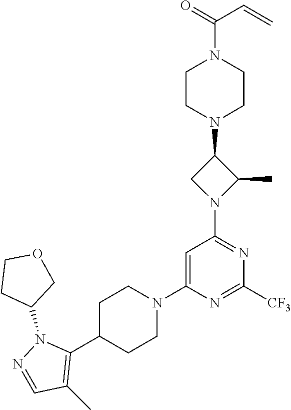 | 1-(4-((2R,3R)-2-methyl- 1-(6-(4-(4-methyl-1- ((R)-tetrahydrofuran-3- yl)-1H-pyrazol-5-yl)- piperidin-1-yl)-2- (trifluoromethyl)pyrimi- din-4-yl)azetidin-3-yl)- piperazin-1-yl)prop-2- en-1-one | E-1 | H-7-2 | 589.3 |
| 2-116 | 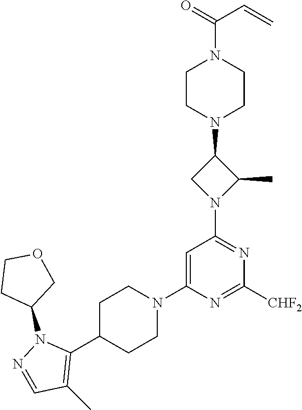 [SMILES: C=CC(=O)N1CCN([C@@H]2CN(c3cc(N4CCC(c5c(C)cnn5[C@H]5CCOC5)CC4)nc(C(F)F)n3)[C@@H]2C)CC1] | 1-(4-((2R,3R)-1-(2- (difluoromethyl)-6-(4- (4-methyl-1-((S)-tetra- hydrofuran-3-yl)-1H- pyrazol-5-yl)piperidin- 1-yl)pyrimidin-4-yl)-2- methylazetidin-3-yl)- piperazin-1-yl)prop-2- en-1-one | E-2 | H-8-1 | 571.3 |
| 2-117 | 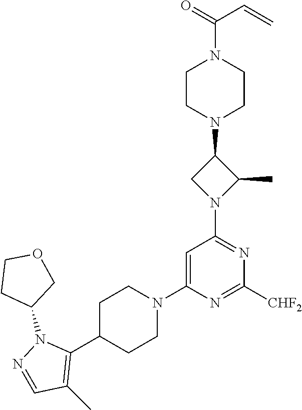 [SMILES: C=CC(=O)N1CCN([C@@H]2CN(c3cc(N4CCC(c5c(C)cnn5[C@@H]5CCOC5)CC4)nc(C(F)F)n3)[C@@H]2C)CC1] | 1-(4-((2R,3R)-1-(2- (difluoromethyl)-6-(4- (4-methyl-1-((R)-tetra- hydrofuran-3-yl)-1H- pyrazol-5-yl)piperidin- 1-yl)pyrimidin-4-yl)-2- methylazetidin-3-yl)- piperazin-1-yl)prop-2- en-1-one | E-9 | H-8-2 | 571.3 |
| 2-118 | 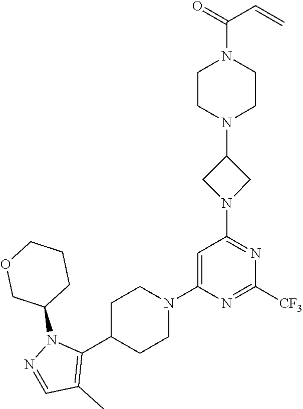 [SMILES: C=CC(=O)N1CCN(C2CN(c3cc(N4CCC(c5c(C)cnn5[C@@H]5CCCOC5)CC4)nc(C(F)(F)F)n3)C2)CC1] | (R)-1-(4-(1-(6-(4-(4- methyl-1-(tetrahydro- 2H-pyran-3-yl)-1H- pyrazol-5-yl)piperidin- 1-yl)-2-(trifluorometh- yl)pyrimidin-4-yl)azeti- din-3-yl)piperazin-1-yl)- prop-2-en-1-one | E-10 | H-9-1 | 589.3 |
| 2-119 | 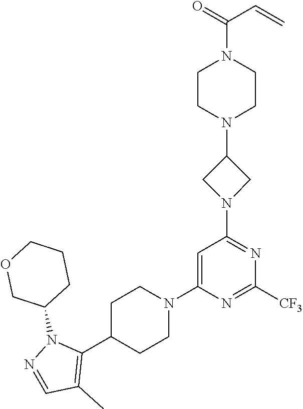 [SMILES: C=CC(=O)N1CCN(C2CN(c3cc(N4CCC(c5c(C)cnn5[C@H]5CCCOC5)CC4)nc(C(F)(F)F)n3)C2)CC1] | (S)-1-(4-(1-(6-(4-(4- methyl-1-(tetrahydro- 2H-pyran-3-yl)-1H- pyrazol-S-yl)piperidin- 1-yl)-2-(trifluorometh- yl)pyrimidin-4-yl)azeti- din-3-yl)piperazin-1-yl)- prop-2-en-1-one | E-10 | H-9-2 | 589.3 |
| 2-120 | 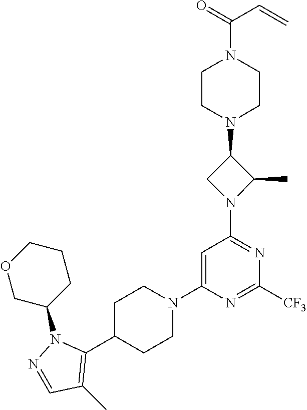 [SMILES: C=CC(=O)N1CCN([C@@H]2CN(c3cc(N4CCC(c5c(C)cnn5[C@@H]5CCCOC5)CC4)nc(C(F)(F)F)n3)[C@@H]2C)CC1] | 1-(4-((2R,3R)-2-methyl- 1-(6-(4-(4-methyl-1- ((R)-tetrahydro-2H- pyran-3-yl)-1H-pyrazol- 5-yl)piperidin-1-yl)-2- (trifluoromethyl)pyrimi- din-4-yl)azetidin-3-yl)- piperazin-1-yl)prop-2- en-1-one | E-9 | H-10-1 | 602.7 |
| 2-121 | 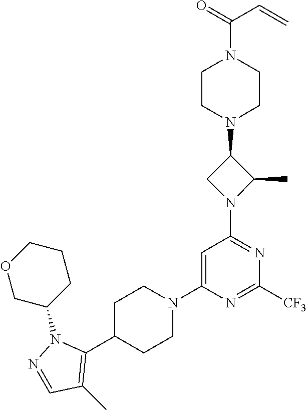 [SMILES: C=CC(=O)N1CCN([C@@H]2CN(c3cc(N4CCC(c5c(C)cnn5[C@H]5CCCOC5)CC4)nc(C(F)(F)F)n3)[C@@H]2C)CC1] | 1-(4-((2R,3R)-2-methyl- 1-(6-(4-(4-methyl-1- ((S)-tetrahydro-2H- pyran-3-yl)-1H-pyrazol- 5-yl)piperidin-1-yl)-2- (trifluoromethyl)pyrimi- din-4-ylazetidin-3-yl)- piperazin-1-yl)prop-2- en-1-one | E-9 | H-10-2 | 602.7 |
| 2-122 | 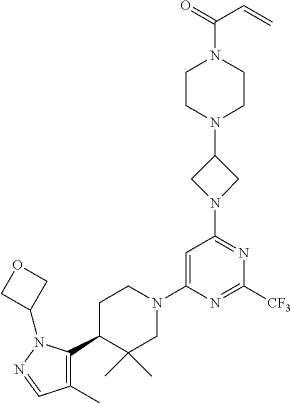 [SMILES: C=CC(=O)N1CCN(C2CN(c3cc(N4CC[C@H](c5c(C)cnn5C5COC5)C(C)(C)C4)nc(C(F)(F)F)n3)C2)CC1] | (S)-1-(4-(1-(6-(3,3- dimethyl-4-(4-methyl-1- (oxetan-3-yl)-1H-pyr- azol-5-yl)piperidin-1-yl)- 2-(trifluoromethyl)pyr- imidin-4-yl)azetidin-3- yl)piperazin-1-yl)prop-2- en-1-one | E-3 | H-11-1 | 588.8 |
| 2-123 | 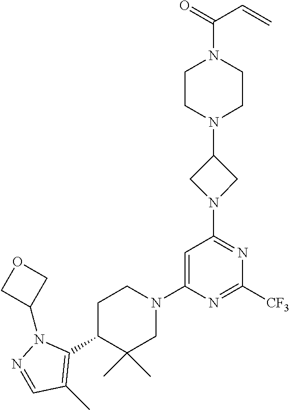 [SMILES: C=CC(=O)N1CCN(C2CN(c3cc(N4CC[C@@H](c5c(C)cnn5C5COC5)C(C)(C)C4)nc(C(F)(F)F)n3)C2)CC1] | (R)-1-(4-(1-(6-(3,3- dimethyl-4-(4-methyl-1- (oxetan-3-yl)-1H-pyr- azol-5-yl)piperidin-1-yl)- 2-(trifluoromethyl)pyr- imidin-4-yl)azetidin-3- yl)pipcrazin-1-yl)prop-2- en-1-one | E-3 | H-11-2 | 588.8 |
| 2-124 | 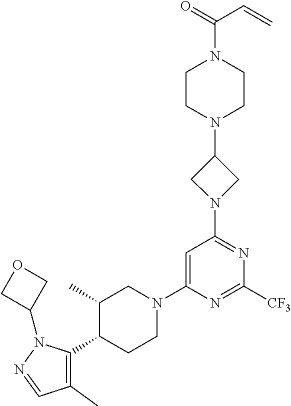 [SMILES: C=CC(=O)N1CCN(C2CN(c3cc(N4CC[C@H](c5c(C)cnn5C5COC5)[C@H](C)C4)nc(C(F)(F)F)n3)C2)CC1] | 1-(4-(1-(6-((35,4S)-3- methyl-4-(4-methyl-1- (oxetan-3-yl)-1H-pyr- azol-5-yl)piperidin-1- yl)-2-(trifluoromethyl)- pyrimidin-4-yl)azetidin- 3-yl)piperazin-1-yl)- prop-2-en-1-one | E-3 | H-12-1 | 575.3 |
| 2-125 | 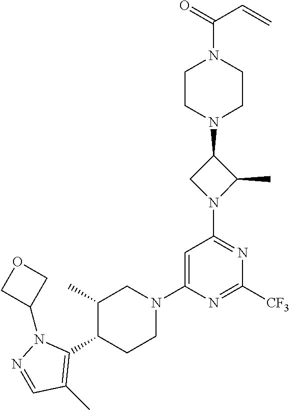 [SMILES: C=CC(=O)N1CCN([C@@H]2CN(c3cc(N4CC[C@H](c5c(C)cnn5C5COC5)[C@H](C)C4)nc(C(F)(F)F)n3)[C@@H]2C)CC1] | 1-(4-((2R,3R)-2-methyl- 1-(6-((35,4S)-3-methyl- 4-(4-methyl-1-(oxetan-3- y])-1H-pyrazol-5-yl)- piperidin-1-yl)-2- (trifluoromethyl)pyrimi- din-4-yl)azetidin-3-yl)- piperazin-1-yl)prop-2- en-1-one | E-1 | H-13-1 | 588.8 |
| 2-126 | 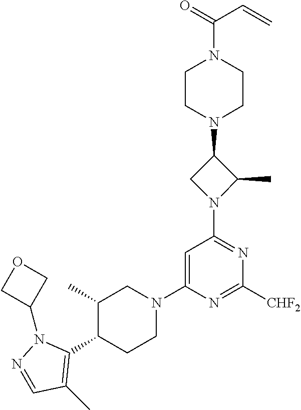 [SMILES: C=CC(=O)N1CCN([C@@H]2CN(c3cc(N4CC[C@H](c5c(C)cnn5C5COC5)[C@H](C)C4)nc(C(F)F)n3)[C@@H]2C)CC1] | 1-(4-((2R,3R)-1-(2- (difluoromethyl)-6- ((3S,4S)-3-methyl-4-(4- methyl-1-(oxetan-3-yl)- 1H-pyrazol-5-yl)piperi- din-1-yl)pyrimidin-4-yl)- 2-methylazetidin-3-yl)- piperazin-1-yl)prop-2- en-1-one | E-2 | H-14-1 | 571.3 |
| 2-127 | 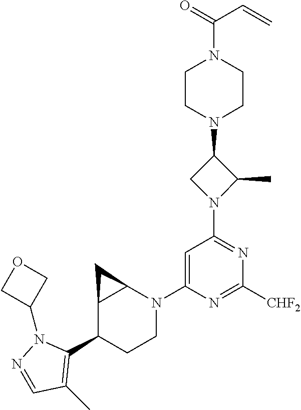 [SMILES: C=CC(=O)N1CCN([C@@H]2CN(c3cc(N4CC[C@@H](c5c(C)cnn5C5COC5)[C@H]5C[C@H]54)nc(C(F)F)n3)[C@@H]2C)CC1] | 1-(4-((2R,3R)-1-(2- (difluoromethyl)-6- ((1R,5R,6R)-5-(4-meth- yl-1-(oxetan-3-yl)-1H- pyrazol-5-yl)-2-azabi- cyclo[4.1.0]heptan-2- yl)pyrimidin-4-yl)-2- methylazetidin-3-yl)- piperazin-1-yl)prop-2- en-1-one | E-2 | H-15-1 | 569.3 |
| 2-128 | 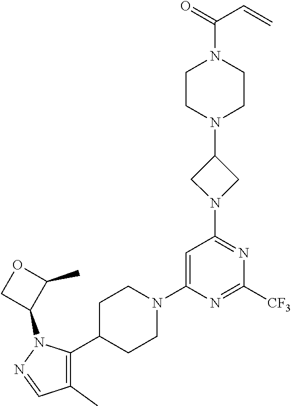 [SMILES: C=CC(=O)N1CCN(C2CN(c3cc(N4CCC(c5c(C)cnn5[C@H]5CO[C@H]5C)CC4)nc(C(F)(F)F)n3)C2)CC1] | 1-(4-(1-(6-(4-(4-methyl- 1-((2S,3S)-2-methyloxe- tan-3-yl)-1H-pyrazol-5- yl)piperidin-1-yl)-2- (trifluoromethyl)pyrimi- din-4-ylazetidin-3-yl)- piperazin-1-yl)prop-2- en-1-one | E-3 | H-16-1 | 575.1 |
| 2-129 | 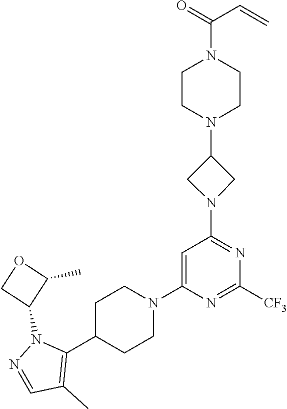 [SMILES: C=CC(=O)N1CCN(C2CN(c3cc(N4CCC(c5c(C)cnn5[C@@H]5CO[C@@H]5C)CC4)nc(C(F)(F)F)n3)C2)CC1] | 1-(4-(1-(6-(4-(4-methyl- 1-((2R.3R)-2-methyl- oxetan-3-yl)-1H-pyrazol- 5-yl)piperidin-1-yl)-2- (trifluoromethyl)pyrimi- din-4-yl)azetidin-3-yl)- piperazin-1-yl)prop-2- en-1-one | E-3 | H-16-2 | 575.1 |
| 2-407 | 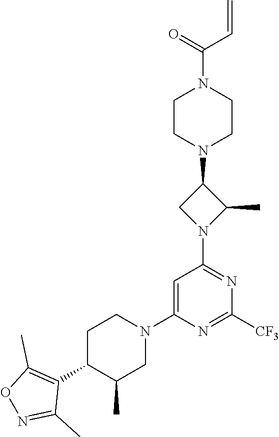 [SMILES: C=CC(=O)N1CCN([C@@H]2CN(c3cc(N4CC[C@@H](c5c(C)noc5C)[C@H](C)C4)nc(C(F)(F)F)n3)[C@@H]2C)CC1] | 1-(4-((2R,3R)-1-(6- ((3S,4R)-4-(3,5-dimeth- yl-1,2-oxazol-4-yl)-3- methyl-1-piperidinyl)-2- (trifluoromethyl)-4- pyrimidinyl)-2-methyl- 3-azetidinyl)-1-piper- azinyl)-2-propen-1-one | E-1 | H-17-1 | 548.0 |
| 2-411 | 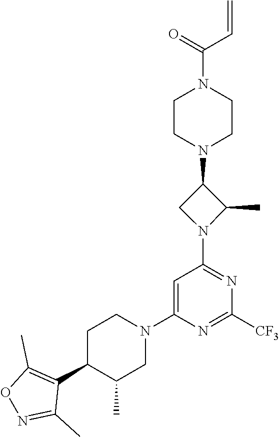 [SMILES: C=CC(=O)N1CCN([C@@H]2CN(c3cc(N4CC[C@H](c5c(C)noc5C)[C@@H](C)C4)nc(C(F)(F)F)n3)[C@@H]2C)CC1] | 1-(4-((2R,3R)-1-(6- ((3R,4S)-4-(3,5-dimeth- yl-1,2-oxazol-4-yl)-3- methyl-1-piperidinyl)-2- (trifluoromethyl)-4- pyrimidinyl)-2-methyl- 3-azetidinyl)-1-piper- azinyl)-2-propen-1-one | E-1 | H-17-2 | 548.0 |
| 2-412 | 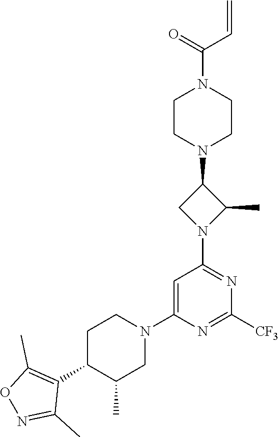 [SMILES: C=CC(=O)N1CCN([C@@H]2CN(c3cc(N4CC[C@@H](c5c(C)noc5C)[C@@H](C)C4)nc(C(F)(F)F)n3)[C@@H]2C)CC1] | 1-(4-((2R,3R)-1-(6- ((3R,4R)-4-(3,5-dimeth- yl-1,2-oxazol-4-yl)-3- methyl-1-piperidinyl)-2- (trifluoromethyl)-4-pyr- imidinyl)-2-methyl-3- azetidinyl)-1-piperazin- yl)-2-propen-1-one | E-1 | H-17-3 | 548.0 |
| 2-415 | 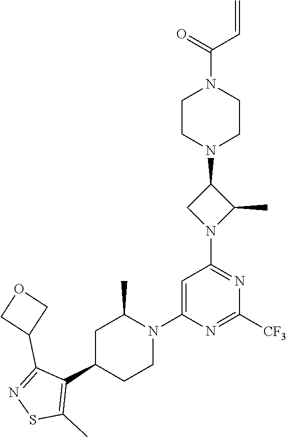 [SMILES: C=CC(=O)N1CCN([C@@H]2CN(c3cc(N4CC[C@@H](c5c(C6COC6)nsc5C)C[C@H]4C)nc(C(F)(F)F)n3)[C@@H]2C)CC1] | 1-(4-((2R,3R)-2-methyl- 1-(6-((2R,4R)-2-methyl- 4-(5-methyl-3-(3-oxetan- yl)-1,2-thiazol-4-yl)-1- piperidinyl)-2-(trifluoro- methyl)-4-pyrimidinyl)- 3-azetidinyl)-1-piper- azinyl)-2-propen-1-one | E-1 | H-18-1 | 606.1 |
| 2-416 | 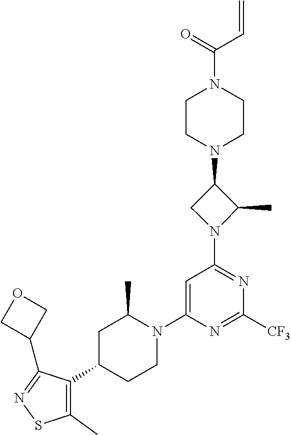 [SMILES: C=CC(=O)N1CCN([C@@H]2CN(c3cc(N4CC[C@H](c5c(C6COC6)nsc5C)C[C@H]4C)nc(C(F)(F)F)n3)[C@@H]2C)CC1] | 1-(4-((2R,3R)-2-methyl- 1-(6-((2R,4S)-2-methyl- 4-(5-methyl-3-(3-oxetan- yl)-1,2-thiazol-4-yl)-1- piperidinyl)-2-(tri- fluoromethyl)-4-pyrimi- dinyl)-3-azetidinyl)-1- piperazinyl)-2-propen- 1-one | E-1 | H-18-2 | 606.1 |
| 2-418 | 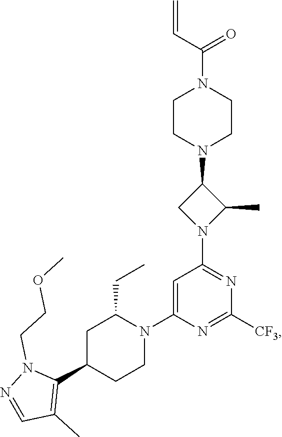 [SMILES: C=CC(=O)N1CCN([C@@H]2CN(c3cc(N4CC[C@@H](c5c(C)cnn5CCOC)C[C@@H]4CC)nc(C(F)(F)F)n3)[C@@H]2C)CC1] | 1-(4-((2R,3R)-1-(6- ((28,4R)-2-ethyl-4-(1-(2- methoxyethyl)-4-methyl- 1H-pyrazol-5-yl)-1- piperidinyl)-2-(trifluoro- methyl)-4-pyrimidinyl)- 2-methyl-3-azetidinyl)- 1-piperazinyl)-2-propen- 1-one | E-8 | H-19-1 | 605.0 |
| 2-419 | 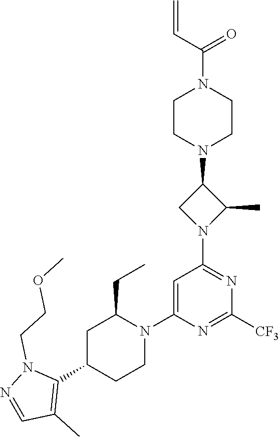 [SMILES: C=CC(=O)N1CCN([C@@H]2CN(c3cc(N4CC[C@H](c5c(C)cnn5CCOC)C[C@H]4CC)nc(C(F)(F)F)n3)[C@@H]2C)CC1] | 1-(4-((2R,3R)-1-(6- ((2R,4S)-2-ethyl-4-(1-(2- methoxyethyl)-4-methyl- 1H-pyrazol-5-yl)-1- piperidinyl)-2-(trifluoro- methyl)-4-pyrimidinyl)- 2-methyl-3-azetidinyl)- 1-piperazinyl)-2-propen- 1-one | E-8 | H-19-2 | 605.0 |
| 2-441 | 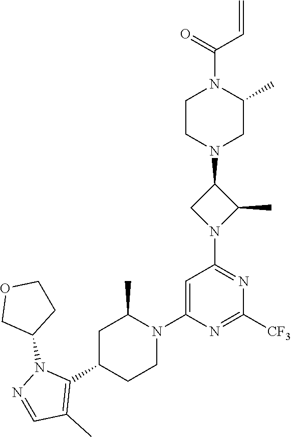 [SMILES: C=CC(=O)N1CCN([C@@H]2CN(c3cc(N4CC[C@H](c5c(C)cnn5[C@H]5CCOC5)C[C@H]4C)nc(C(F)(F)F)n3)[C@@H]2C)C[C@H]1C] | 1-((2R)-2-methyl-4- ((2R,3R)-2-methyl-1-(6- ((2R,4S)-2-methyl-4-(4- methyl-1-((3S)-tetra- hydro-3-furanyl)-1H- pyrazol-5-yl)-1-piperi- dinyl)-2-(trifluorometh- yl)-4-pyrimidinyl)-3- azetidinyl)-1-piperazin- yl)-2-propen-1-one | E-4 | H-20-1 | 616.9 |
| 2-442 | 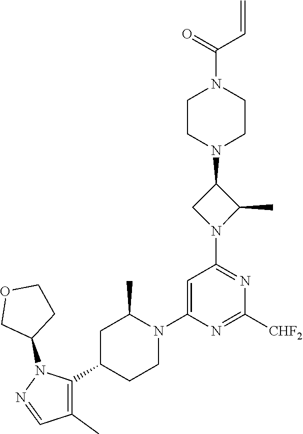 [SMILES: C=CC(=O)N1CCN([C@@H]2CN(c3cc(N4CC[C@H](c5c(C)cnn5[C@@H]5CCOC5)C[C@H]4C)nc(C(F)F)n3)[C@@H]2C)CC1] | 1-(4-((2R,3R)-1-(2- (difluoromethyl)-6- ((2R,4S)-2-methyl-4-(4- methyl-1-((3R)-tetra- hydro-3-furanyl)-1H- pyrazol-5-yl)-1-piperi- dinyl)-4-pyrimidinyl)-2- methyl-3-azetidinyl)-1- piperazinyl)-2-propen- 1-one | E-2 | H-21-1 | 585.0 |
| 2-444 | 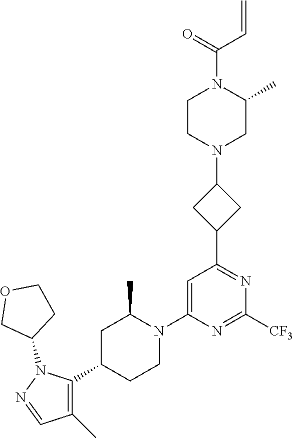 [SMILES: C=CC(=O)N1CCN(C2CC(c3cc(N4CC[C@H](c5c(C)cnn5[C@H]5CCOC5)C[C@H]4C)nc(C(F)(F)F)n3)C2)C[C@H]1C] | 1-((2R)-2-methyl-4-(1-(6- ((2R,4S)-2-methyl-4-(4- methyl-1-((3S)-tetra- hydro-3-furanyl)-1H-pyr- azol-5-yl)-1-piperidinyl)- 2-(trifluoromethyl)-4- pyrimidinyl)-3-azetidin- yl)-1-piperazinyl)-2- propen-1-one | E-4 | H-22-2 | 602.95 |
| 2-447 | 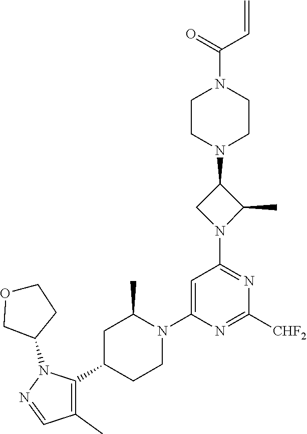 [SMILES: C=CC(=O)N1CCN([C@@H]2CN(c3cc(N4CC[C@H](c5c(C)cnn5[C@H]5CCOC5)C[C@H]4C)nc(C(F)F)n3)[C@@H]2C)CC1] | 1-(4-((2R,3R)-1-(2- (difluoromethyl)-6- ((2R,4S)-2-methyl-4-(4- methyl-1-((3S)-tetra- hydro-3-furanyl)-1H- pyrazol-5-yl)-1-piperi- dinyl)-4-pyrimidinyl)-2- methyl-3-azetidinyl)-1- piperazinyl)-2-propen- 1-one | E-8 | H-21-2 | 585.0 |
| 2-448 | 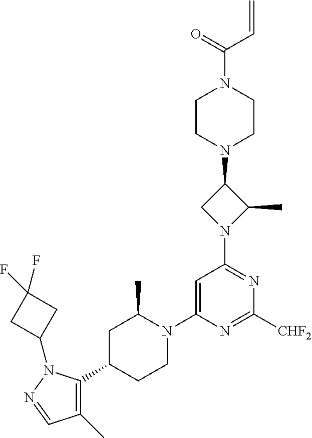 [SMILES: C=CC(=O)N1CCN([C@@H]2CN(c3cc(N4CC[C@H](c5c(C)cnn5C5CC(F)(F)C5)C[C@H]4C)nc(C(F)F)n3)[C@@H]2C)CC1] | 1-(4-((2R,3R)-1-(6- ((2R,4S)-4-(1-(3,3- difluorocyclobutyl])-4- methyl-1H-pyrazol-5- yl)-2-methyl-1-piperidin- yl)-2-(difluoromethyl)-4- pyrimidinyl)-2-methyl-3- azetidinyl)-1-piperazin- yl)-2-propen-1-one | E-9 | H-23-2 | 605.0 |
| 2-449 | 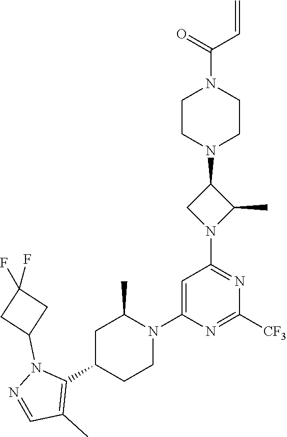 [SMILES: C=CC(=O)N1CCN([C@@H]2CN(c3cc(N4CC[C@H](c5c(C)cnn5C5CC(F)(F)C5)C[C@H]4C)nc(C(F)(F)F)n3)[C@@H]2C)CC1] | 1-(4-((2R,3R)-1-(6- ((2R,4S)-4-(1-(3,3- difluorocyclobutyl)-4- methyl-1H-pyrazol-5-yl)- 2-methyl-1-piperidinyl)- 2-(trifluoromethyl)-4- pyrimidinyl)-2-methyl- 3-azetidinyl)-1-piperazin- yl)-2-propen-1-one | E-8 | H-24-2 | 623.0 |
| 2-452 | 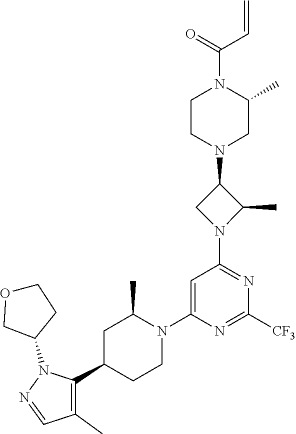 [SMILES: C=CC(=O)N1CCN([C@@H]2CN(c3cc(N4CC[C@@H](c5c(C)cnn5[C@H]5CCOC5)C[C@H]4C)nc(C(F)(F)F)n3)[C@@H]2C)C[C@H]1C] | 1-((2R)-2-methyl-4- ((2R,3R)-2-methyl-1-(6- ((2R,4R)-2-methyl-4-(4- methyl-1-((3S)-tetra- hydro-3-furanyl)-1H- pyrazol-5-yl)-1-piperi- dinyl)-2-(trifluorometh- yl)-4-pyrimidinyl)-3- azetidinyl)-1-piperazin- yl)-2-propen-1-one | E-6 | H-28-1 | 617.0 |
| 2-453 | 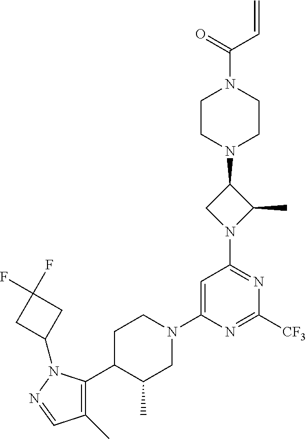 [SMILES: C=CC(=O)N1CCN([C@@H]2CN(c3cc(N4CCC(c5c(C)cnn5C5CC(F)(F)C5)[C@@H](C)C4)nc(C(F)(F)F)n3)[C@@H]2C)CC1] | 1-(4-((2R,3R)-1-(6- ((3R)-4-(1-(3,3-difluoro- cyclobutyl)-4-methyl- 1H-pyrazol-5-yl)-3- methylpiperidin-1-yl)- 2-(trifluoromethyl)pyr- imidin-4-yl)-2-methyl- azetidin-3-yl)piperazin- 1-yl)prop-2-en-1-one | E-8 | H-24-1; H-24-2 | 623.0 |
| 2-456 | 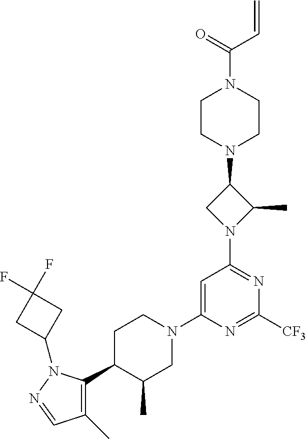 [SMILES: C=CC(=O)N1CCN([C@@H]2CN(c3cc(N4CC[C@H](c5c(C)cnn5C5CC(F)(F)C5)[C@H](C)C4)nc(C(F)(F)F)n3)[C@@H]2C)CC1] | 1-(4-((2R,3R)-1-(6- ((3S,4S)-4-(1-(3,3- difluorocyclobutyl)-4- methyl-1H-pyrazol-5- yl)-3-methyl-1-piperi- dinyl)-2-(trifluorometh- yl)-4-pyrimidinyl)-2- methyl-3-azetidinyl)-1- piperazinyl)-2-propen- 1-one | E-8 | H-25-4 | 622.9 |
| 2-458 | 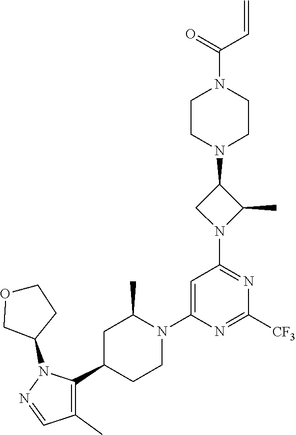 [SMILES: C=CC(=O)N1CCN([C@@H]2CN(c3cc(N4CC[C@@H](c5c(C)cnn5[C@@H]5CCOC5)C[C@H]4C)nc(C(F)(F)F)n3)[C@@H]2C)CC1] | 1-(4-((2R,3R)-2-methyl- 1-(6-((2R,4R)-2-methyl- 4-(4-methyl]-1-((3R)- tetrahydro-3-furanyl)- 1H-pyrazol-5-yl)-1- piperidinyl)-2-(trifluoro- methyl)-4-pyrimidinyl)- 3-azetidinyl)-1-piper- azinyl)-2-propen-1-one | E-8 | H-26-2 | 603.0 |
| 2-460 | 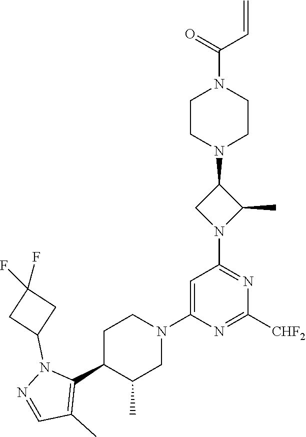 [SMILES: C=CC(=O)N1CCN([C@@H]2CN(c3cc(N4CC[C@H](c5c(C)cnn5C5CC(F)(F)C5)[C@@H](C)C4)nc(C(F)F)n3)[C@@H]2C)CC1] | 1-(4-((2R,3R)-1-(6- ((3R,4S)-4-(1-(3,3- difluorocyclobutyl)-4- methyl-IH-pyrazol-5-yl)- 3-methyl-1-piperidinyl)- 2-(difluoromethyl)-4- pyrimidinyl)-2-methyl- 3-azetidinyl)-1-piper- azinyl)-2-propen-1-one | E-9 | H-27-3 | 604.9 |
| 2-461 | 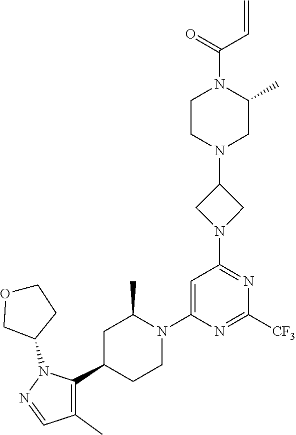 [SMILES: C=CC(=O)N1CCN(C2CN(c3cc(N4CC[C@@H](c5c(C)cnn5[C@H]5CCOC5)C[C@H]4C)nc(C(F)(F)F)n3)C2)C[C@H]1C] | 1-((2R)-2-methyl-4-(1- (6-((2R,4R)-2-methyl- 4-(4-methyl-1-((3S)- tetrahydro-3-furanyl)- 1H-pyrazol-5-yl)-1- piperidinyl)-2-(trifluoro- methyl)-4-pyrimidinyl)- 3-azetidinyl)-1]-piper- azinyl)-2-propen-1-one | E-4 | H-22-1 | 603.0 |
| 2-465 | 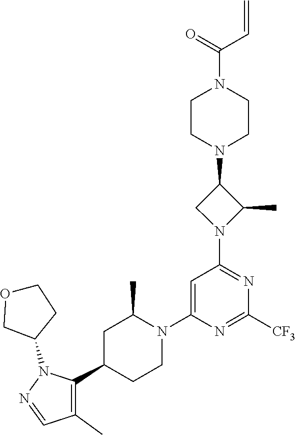 [SMILES: C=CC(=O)N1CCN([C@@H]2CN(c3cc(N4CC[C@@H](c5c(C)cnn5[C@H]5CCOC5)C[C@H]4C)nc(C(F)(F)F)n3)[C@@H]2C)CC1] | 1-(4-((2R,3R)-2-methyl- 1-(6-((2R,4R)-2-methyl- 4-(4-methyl-1-((3S)- tetrahydro-3-furanyl)- 1H-pyrazol-5-yl)-1- piperidinyl)-2-(trifluoro- methyl)-4-pyrimidinyl)- 3-azetidinyl)-1-piper- azinyl)-2-propen-1-one | E-8 | H-26-1 | 603.0 |
| 2-467 | 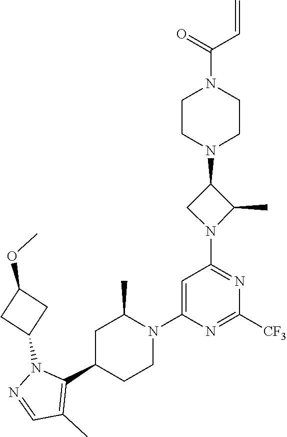 [SMILES: C=CC(=O)N1CCN([C@@H]2CN(c3cc(N4CC[C@@H](c5c(C)cnn5[C@H]5C[C@H](OC)C5)C[C@H]4C)nc(C(F)(F)F)n3)[C@@H]2C)CC1] | 1-(4-((2R,3R)-1-(6- ((2R,4R)-4-(1-(trans-3- methoxycyclobutyl)-4- methyl-1H-pyrazol-5- yl)-2-methyl-1-piperidin- yl)-2-(trifluoromethyl)- 4-pyrimidinyl)-2-methyl- 3-azetidinyl)-1-piperazin- yl)-2-propen-1-one | E-8 | H-29-1 | 617.1 |
| 2-468 | 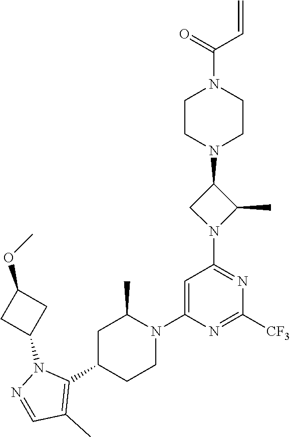 [SMILES: C=CC(=O)N1CCN([C@@H]2CN(c3cc(N4CC[C@H](c5c(C)cnn5[C@H]5C[C@H](OC)C5)C[C@H]4C)nc(C(F)(F)F)n3)[C@@H]2C)CC1] | 1-(4-((2R,3R)-1-(6- ((2R,4S)-4-(1-(trans-3- methoxycyclobutyl)- 4-methyl-1H-pyrazol-5- yl)-2-methyl-1-piperidin- yl)-2-(trifluoromethyl)- 4-pyrimidinyl)-2-methyl- 3-azetidinyl)-1-piper- azinyl)-2-propen-1-one | E-8 | H-29-2 | 617.1 |
| 2-500 | 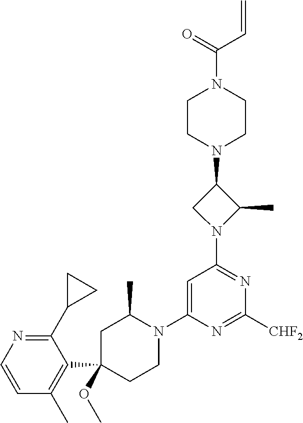 [SMILES: C=CC(=O)N1CCN([C@@H]2CN(c3cc(N4CC[C@](OC)(c5c(C)ccnc5C5CC5)C[C@H]4C)nc(C(F)F)n3)[C@@H]2C)CC1] | 1-(4-((2R,3R)-1-(6- ((2R,4R)-4-(2-cyclo- propyl-4-methyl-3- pyridinyl)-4-methoxy- 2-methyl-1-piperidinyl)- 2-(difluoromethyl)-4- pyrimidinyl)-2-methyl- 3-azetidinyl)-1-piper- azinyl)-2-propen-1-one | E-9 | H-30-1 | 603.0 |
| 2-513 | 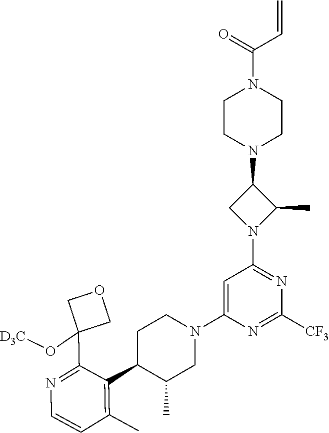 [SMILES: [2H]C([2H])([2H])OC1(c2nccc(C)c2[C@H]2CCN(c3cc(N4C[C@@H](N5CCN(C(=O)C=C)CC5)[C@H]4C)nc(C(F)(F)F)n3)C[C@@H]2C)COC1] | 1-(4-((2R,3R)-1-(6- ((3R,4S)-4-(2-(3-(meth- oxy-d₃)oxetan-3-yl)-4- methylpyridin-3-yl)-3- methylpiperidin-1-yl)-2- (trifluoromethyl)pyrimi- din-4-yl)-2-methylazeti- din-3-yl)piperazin-1-yl)- prop-2-en-1-one | E-8 | H-31-2 | 633.0 |
| 2-524 | 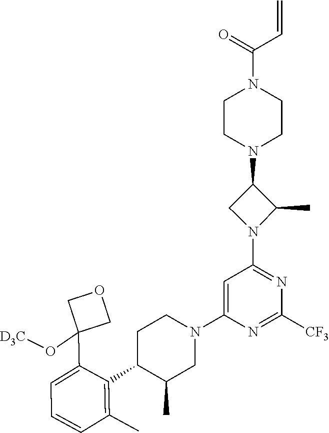 [SMILES: [2H]C([2H])([2H])OC1(c2cccc(C)c2[C@@H]2CCN(c3cc(N4C[C@@H](N5CCN(C(=O)C=C)CC5)[C@H]4C)nc(C(F)(F)F)n3)C[C@H]2C)COC1] | 1-(4-((2R,3R)-1-(6- ((3S,4R)-4-(2-(3-meth- oxy-d₃)oxetan-3-yl)-4- methylpyridin-3-yl)-3- methylpiperidin-1-yl)-2- (trifluoromethyl)pyrimi- din-4-yl)-2-methylazeti- din-3-yl)piperazin-1-yl)- prop-2-en-1-one | E-8 | H-31-1 | 633.0 |
| 2-557 | 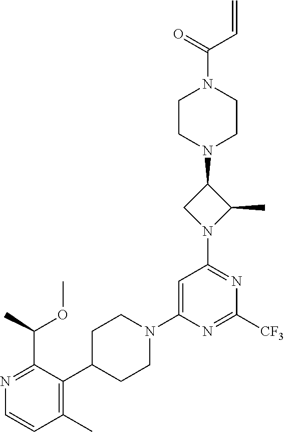 [SMILES: C=CC(=O)N1CCN([C@@H]2CN(c3cc(N4CCC(c5c(C)ccnc5[C@@H](C)OC)CC4)nc(C(F)(F)F)n3)[C@@H]2C)CC1] | 1-(4-((2R,3R)-1-(6-(4- (2-((1R)-1-methoxyeth- yl)-4-methyl-3-pyridin- yl)-1-piperidinyl)-2- (trifluoromethyl)-4- pyrimidinyl)-2-methyl- 3-azetidinyl)-1-piper- azinyl)-2-propen-1-one | E-1 | H-32-1 | 588.4 |
| 2-558 | 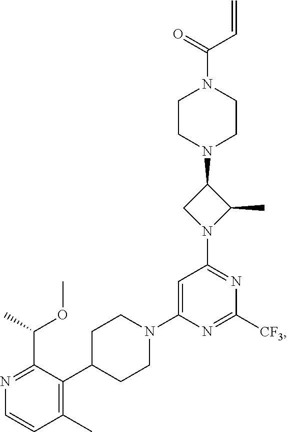 [SMILES: C=CC(=O)N1CCN([C@@H]2CN(c3cc(N4CCC(c5c(C)ccnc5[C@H](C)OC)CC4)nc(C(F)(F)F)n3)[C@@H]2C)CC1] | 1-(4-((2R,3R)-1-(6-(4- (2-((1S)-1-ethoxyethyl)- 4-methyl-3-pyridinyl)-1- piperidinyl)-2-(trifluoro- methyl)-4-pyrimidinyl)- 2-methyl-3-azetidinyl)- 1-piperazinyl)-2-propen- 1-one | E~1 | H-32-2 | 588.5 |
| 2-571 | 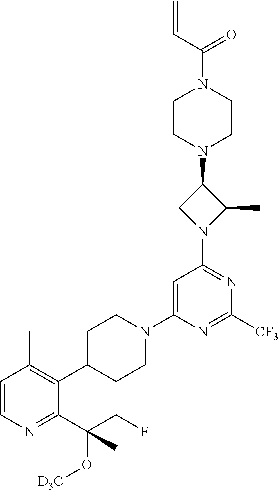 [SMILES: [2H]C([2H])([2H])O[C@@](C)(CF)c1nccc(C)c1C1CCN(c2cc(N3C[C@@H](N4CCN(C(=O)C=C)CC4)[C@H]3C)nc(C(F)(F)F)n2)CC1] | 1-(4-((2R,3R)-1-(6-(4- (2-((R)-1-fluoro-2- (methoxy-d₃)propan-2- yl)-4-methylpyridin-3- yl)piperidin-1-yl)-2- (trifluoromethyl)pyrimi- din-4-yl)-2-methylazeti- din-3-yl)piperazin-1-yl)- prop-2-en-1-one | E-1 | H-33-1 | 623.4 |
| 2-572 | 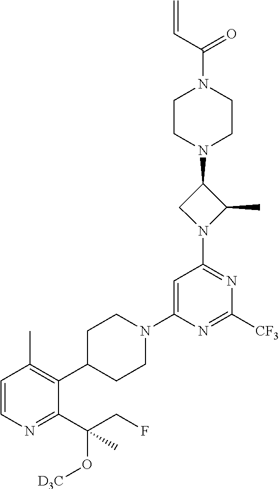 [SMILES: [2H]C([2H])([2H])O[C@](C)(CF)c1nccc(C)c1C1CCN(c2cc(N3C[C@@H](N4CCN(C(=O)C=C)CC4)[C@H]3C)nc(C(F)(F)F)n2)CC1] | 1-(4-((2R,3R)-1-(6-(4- (2-((S)-1-fluoro-2- (methoxy-d₃)propan-2- yl)-4-methylpyridin-3- yl)piperidin-1-yl)-2- (trifluoromethyl)pyrimi- din-4-yl)-2-methylazeti- din-3-yl)piperazin-1-yl)- prop-2-en-1-one | E-1 | H-33-2 | 623.4 |
| 2-573 | 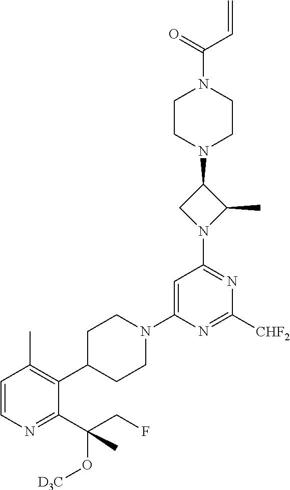 [SMILES: [2H]C([2H])([2H])O[C@@](C)(CF)c1nccc(C)c1C1CCN(c2cc(N3C[C@@H](N4CCN(C(=O)C=C)CC4)[C@H]3C)nc(C(F)F)n2)CC1] | 1-(4-((2R,3R)-1-(2- (difluoromethyl)-6-(4- (2-((R)-1-fluoro-2- (methoxy-d₃)propan-2- yl)-4-methylpyridin-3- yl)piperidin-1-yl)pyrimi- din-4-yl)-2-methylazeti- din-3-yl)piperazin-1-yl)- prop-2-en-1-one | E~8 | H-34-1 | 605.4 |
| 2-574 | 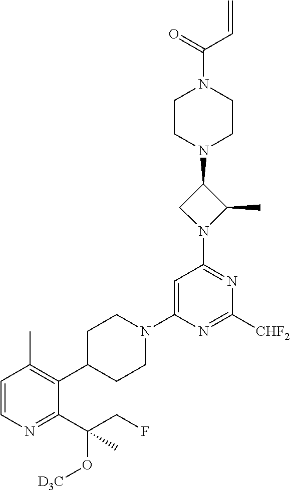 [SMILES: [2H]C([2H])([2H])O[C@](C)(CF)c1nccc(C)c1C1CCN(c2cc(N3C[C@@H](N4CCN(C(=O)C=C)CC4)[C@H]3C)nc(C(F)F)n2)CC1] | 1-(4-((2R,3R)-1-(2- (difluoromethyl)-6-(4- (2-((S)-1-fluoro-2- (methoxy-d₃)propan-2- yl)-4-methylpyridin-3-yl)- piperidin-1-yl)pyrimidin- 4-yl)-2-methylazetidin-3- yl)piperazin-1-yl)prop-2- en-1-one | E-8 | H-34-2 | 605.4 |
| 2-575 | 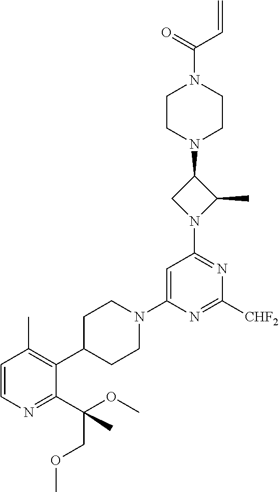 [SMILES: C=CC(=O)N1CCN([C@@H]2CN(c3cc(N4CCC(c5c(C)ccnc5[C@@](C)(COC)OC)CC4)nc(C(F)F)n3)[C@@H]2C)CC1] | 1-(4-((2R,3R)-1-(2- (difluoromethyl)-6-(4-(2- ((2S)-1,2-dimethoxy-2- propanyl)-4-methyl-3- pyridinyl)-1-piperidin- yl)-4-pyrimidinyl)-2- methyl-3-azetidinyl)-1- piperazinyl)-2-propen- 1-one | E-8 | H-35-1 | 614.3 |
| 2-576 | 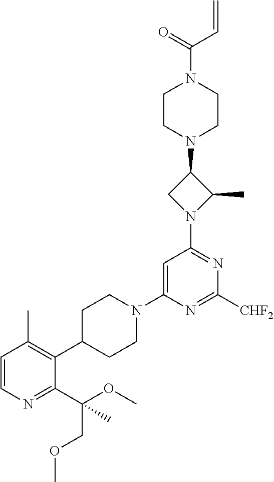 [SMILES: C=CC(=O)N1CCN([C@@H]2CN(c3cc(N4CCC(c5c(C)ccnc5[C@](C)(COC)OC)CC4)nc(C(F)F)n3)[C@@H]2C)CC1] | 1-(4-((2R,3R)-1-(2- (difluoromethyl)-6-(4-(2- (2R)-1,2-dimethoxy-2- propanyl)-4-methyl-3- pyridinyl)-1-piperidin- yl)-4-pyrimidinyl)-2- methyl-3-azetidinyl)-1- piperazinyl)-2-propen- 1-one | E-8 | H-35-2 | 614.3 |
| 2-577 | 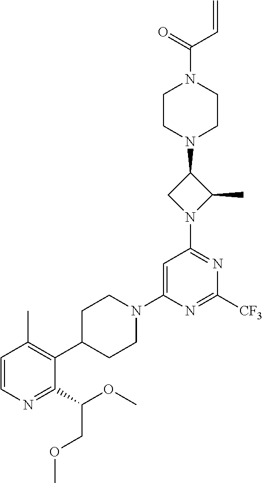 [SMILES: C=CC(=O)N1CCN([C@@H]2CN(c3cc(N4CCC(c5c(C)ccnc5[C@@H](COC)OC)CC4)nc(C(F)(F)F)n3)[C@@H]2C)CC1] | 1-(4-((2R,3R)-1-(6-(4-(2- ((1S)-1,2-dimethoxyeth- yl)-4-methyl-3-pyridin- yl)-1-piperidinyl)-2- (trifluoromethyl)-4- pyrimidinyl)-2-methyl- 3-azetidinyl)-1-piperazin- yl)-2-propen-1-one | E-1 | H-36-1 | 618.3 |
| 2-578 | 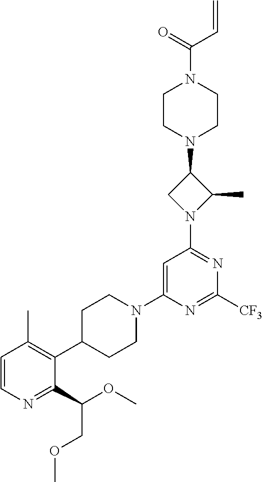 [SMILES: C=CC(=O)N1CCN([C@@H]2CN(c3cc(N4CCC(c5c(C)ccnc5[C@H](COC)OC)CC4)nc(C(F)(F)F)n3)[C@@H]2C)CC1] | 1-(4-((2R,3R)-1-(6-(4- (2-((1R)-1,2-dimethoxy- ethyl)-4-methyl-3-pyr- idinyl)-1-piperidinyl)-2- (trifluoromethyl)-4- pyrimidinyl)-2-methyl- 3-azetidinyl)-1-piper- azinyl)-2-propen-1-one | E-1 | H-36-2 | 618.4 |
| 2-579 | 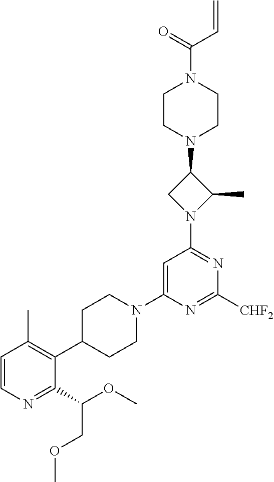 [SMILES: C=CC(=O)N1CCN([C@@H]2CN(c3cc(N4CCC(c5c(C)ccnc5[C@@H](COC)OC)CC4)nc(C(F)F)n3)[C@@H]2C)CC1] | 1-(4-((2R,3R)-1-(2- (difluoromethyl)-6-(4- (2-((1S)-1,2-dimethoxy- ethyl)-4-methyl-3- pyridinyl)-1-piperidin- yl)-4-pyrimidinyl)-2- methyl-3-azetidinyl)-1- piperazinyl)-2-propen- 1-one | E-9 | H-37-1 | 600.3 |
| 2-580 | 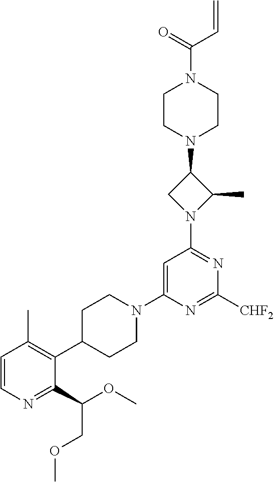 [SMILES: C=CC(=O)N1CCN([C@@H]2CN(c3cc(N4CCC(c5c(C)ccnc5[C@H](COC)OC)CC4)nc(C(F)F)n3)[C@@H]2C)CC1] | 1-(4-((2R,3R)-1-(2- (difluoromethyl)-6-(4- (2-((1R)-1,2-dimethoxy- ethyl)-4-methyl-3-pyr- idinyl)-1-piperidinyl)-4- pyrimidinyl)-2-methyl-3- azetidinyl)-1-piperazin- yl)-2-propen-1-one | E-9 | H-37-2 | 600.4 |
| 2-581 | 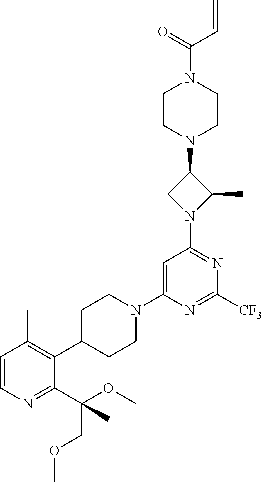 [SMILES: C=CC(=O)N1CCN([C@@H]2CN(c3cc(N4CCC(c5c(C)ccnc5[C@@](C)(COC)OC)CC4)nc(C(F)(F)F)n3)[C@@H]2C)CC1] | 1-(4-((2R,3R)-1-(6-(4- (2-((2S)-1,2-dimethoxy- 2-propanyl)-4-methyl-3- pyridinyl)-1-piperidin- yl)-2-(trifluoromethyl)- 4-pyrimidinyl)-2-methyl- 3-azetidinyl)-1-piperazin- yl)-2-propen-1-one | E-1 | H-38-1 | 632.4 |
| 2-582 | 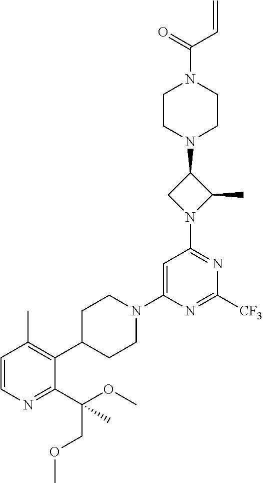 [SMILES: C=CC(=O)N1CCN([C@@H]2CN(c3cc(N4CCC(c5c(C)ccnc5[C@](C)(COC)OC)CC4)nc(C(F)(F)F)n3)[C@@H]2C)CC1] | 1-(4-((2R,3R)-1-(6-(4- (2-((2R)-1,2-dimethoxy- 2-propanyl)-4-methyl-3- pyridinyl)-1-piperidin- yl)-2-(trifluoromethyl)- 4-pyrimidinyl)-2-methyl- 3-azetidinyl)-1-piperazin- yl)-2-propen-1-one | E-1 | H-38-2 | 632.4 |
| 2-588 | 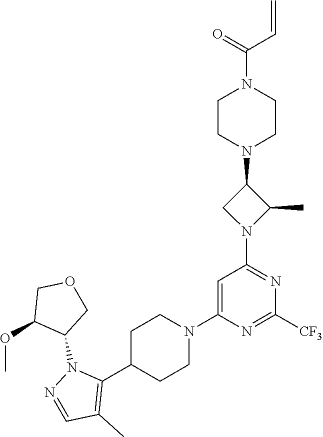 [SMILES: C=CC(=O)N1CCN([C@@H]2CN(c3cc(N4CCC(c5c(C)cnn5[C@H]5COC[C@@H]5OC)CC4)nc(C(F)(F)F)n3)[C@@H]2C)CC1] | 1-(4-((2R,3R)-1-(6-(4- (1-((3S,4R)-4-methoxy- tetrahydro-3-furanyl)-4- methyl-1H-pyrazol-5- yl)-1-piperidinyl)-2- (trifluoromethyl)-4-pyr- imidinyl)-2-methyl-3- azetidinyl)-1-piperazin- yl)-2-propen-1-one | E-1 | H-39-1 | 619.2 |
| 2-589 | 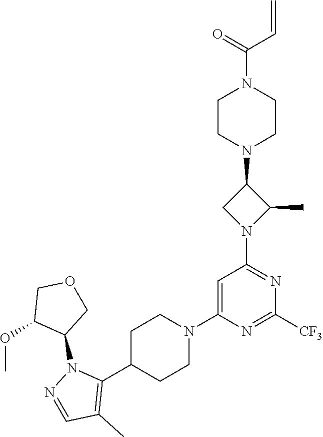 [SMILES: C=CC(=O)N1CCN([C@@H]2CN(c3cc(N4CCC(c5c(C)cnn5[C@@H]5COC[C@H]5OC)CC4)nc(C(F)(F)F)n3)[C@@H]2C)CC1] | 1-(4-((2R,3R)-1-(6-(4- (1-((3R,4S)-4-methoxy- tetrahydro-3-furanyl)-4- methyl-1H-pyrazol-5- yl)-1-piperidinyl)-2- (trifluoromethyl)-4- pyrimidinyl)-2-methyl- 3-azetidinyl)-1-piper- azinyl)-2-propen-1-one | E-1 | H-39-2 | 619.2 |
| 2-592 | 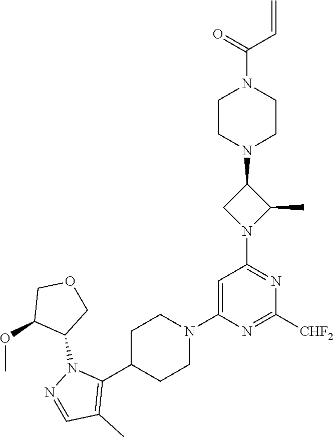 [SMILES: C=CC(=O)N1CCN([C@@H]2CN(c3cc(N4CCC(c5c(C)cnn5[C@H]5COC[C@@H]5OC)CC4)nc(C(F)F)n3)[C@@H]2C)CC1] | 1-(4-((2R,3R)-1-(2- (difluoromethyl)-6-(4- (1-((3S.4R)-4-methoxy- tetrahydro-3-furanyl)-4- methyl-1H-pyrazol-5- yl)-1-piperidinyl)-4- pyrimidinyl)-2-methyl- 3-azetidinyl)-1-piper- azinyl)-2-propen-1-one | E-9 | H-40-1 | 601.3 |
| 2-593 | 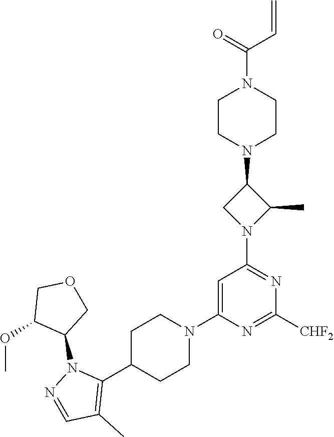 [SMILES: C=CC(=O)N1CCN([C@@H]2CN(c3cc(N4CCC(c5c(C)cnn5[C@@H]5COC[C@H]5OC)CC4)nc(C(F)F)n3)[C@@H]2C)CC1] | 1-(4-((2R,3R)-1-(2- (difluoromethyl)-6-(4- (1-((3R,4S)-4-methoxy- tetrahydro-3-furanyl)-4- methyl-1H-pyrazol-5- yl)-1-piperidinyl)-4- pyrimidinyl)-2-methyl- 3-azetidinyl)-1-piper- azinyl)-2-propen-1-one | E-88 | H-40-2 | 601.2 |
| 2-632 | 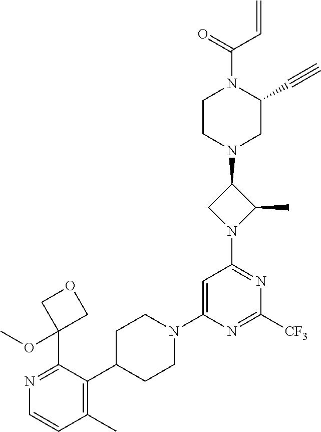 [SMILES: C#C[C@@H]1CN([C@@H]2CN(c3cc(N4CCC(c5c(C)ccnc5C5(OC)COC5)CC4)nc(C(F)(F)F)n3)[C@@H]2C)CCN1C(=O)C=C] | 1-((2R)-2-ethynyl-4- ((2R,3R)-1-(6-(4-(2-(3- methoxy-3-oxetanyl)-4- methyl-3-pyridinyl)-1- piperidinyl)-2-(trifluoro- methyl)-4-pyrimidinyl)- 2-methyl-3-azetidinyl)- 1-piperazinyl)-2-propen- 1-one | E-12 | H-41-1 | 640.2 |
| 2-633 | 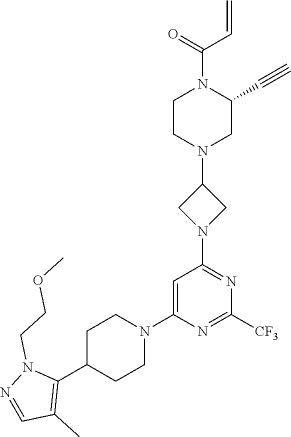 [SMILES: C#C[C@@H]1CN(C2CN(c3cc(N4CCC(c5c(C)cnn5CCOC)CC4)nc(C(F)(F)F)n3)C2)CCN1C(=O)C=C] | 1-((2R)-2-ethynyl-4-(1- (6-(4-(1-(2-methoxyeth- yl)-4-methyl-1H-pyrazol- 5-yl)-1-piperidinyl)-2- (trifluoromethyl)-4- pyrimidinyl)-3-azetidin- yl)-1-piperazinyl)-2- propen-1-one | E-12 | H-42-1 | 586.8 |
| 2-634 | 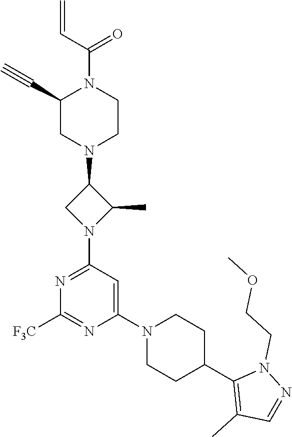 [SMILES: C#C[C@@H]1CN([C@@H]2CN(c3cc(N4CCC(c5c(C)cnn5CCOC)CC4)nc(C(F)(F)F)n3)[C@@H]2C)CCN1C(=O)C=C] | 1-((2R)-2-ethynyl-4- ((2R,3R)-1-(6-(4-(1-(2- methoxyethyl)-4-methyl- 1H-pyrazol-5-yl)-1- piperidinyl)-2-(trifluoro- methyl)-4-pyrimidinyl)- 2-methyl-3-azetidinyl)- 1-piperazinyl)-2-propen- 1-one | E-12 | H-43-2 | 601.2 |
| 2-635 (mix) | 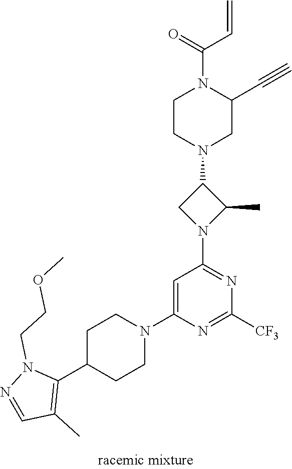 | 1-(2-ethynyl-4-((2R,3S)- 1-(6-(4-(1-(2-methoxy- ethyl)-4-methyl-1H-pyr- azol-5-yl)-1-piperidinyl)- 2-(trifluoromethyl)-4- pyrimidinyl)-2-methyl- 3-azetidinyl)-1-piper- azinyl)-2-propen-1-one | E-12 | H-43-1 | 601.2 |
| 2-636 | 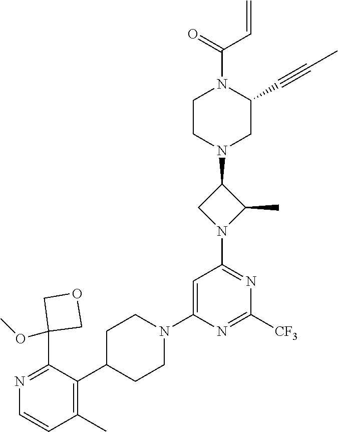 [SMILES: C=CC(=O)N1CCN([C@@H]2CN(c3cc(N4CCC(c5c(C)ccnc5C5(OC)COC5)CC4)nc(C(F)(F)F)n3)[C@@H]2C)C[C@H]1C#CC] | 1-((2R)-4-((2R,3R)-1-(6- (4-(2-(3-methoxy-3-oxe- tanyl)-4-methyl-3-pyr- idinyl)-1-piperidinyl)-2- (trifluoromethyl)-4-pyr- imidinyl)-2-methyl-3- azetidinyl)-2-(1-propyn- 1-yl)-1-piperazinyl)-2- propen-1-one | E-12 | H-44-1 | 654.1 |
| 2-637 | 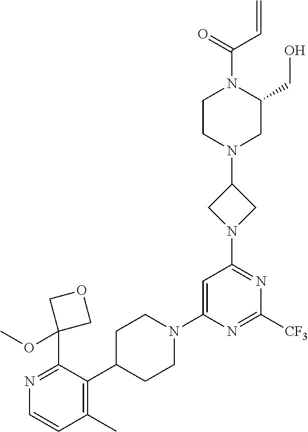 [SMILES: C=CC(=O)N1CCN(C2CN(c3cc(N4CCC(c5c(C)ccnc5C5(OC)COC5)CC4)nc(C(F)(F)F)n3)C2)C[C@H]1CO] | 1-((2S)-2-hydroxymeth- yl)-4-(1-(6-(4-(2-(3- methoxy-3-oxetanyl)-4- methyl-3-pyridinyl)-1- piperidinyl)-2-(trifluoro- methyl)-4-pyrimidinyl)- 3-azetidinyl)-1-piper- azinyl)-2-propen-1-one | E-13 | H-45-1 | 632.2 |
| 2-638 | 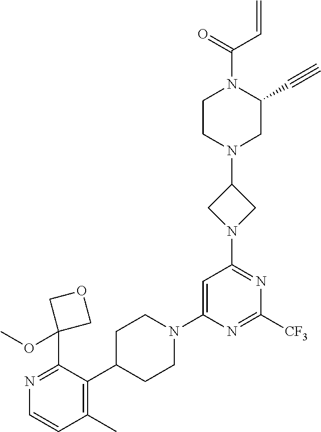 [SMILES: C#C[C@@H]1CN(C2CN(c3cc(N4CCC(c5c(C)ccnc5C5(OC)COC5)CC4)nc(C(F)(F)F)n3)C2)CCN1C(=O)C=C] | 1-((2R)-2-ethynyl-4-(1- (6-(4-(2-(3-methoxy-3- oxetanyl)-4-methyl-3- pyridinyl)-1-piperidin- yl)-2-(trifluoromethyl)- 4-pyrimidinyl)-3-azeti- dinyl)-1-piperazinyl)-2- propen-1-one | E-12 | H-46-1 | 626.4 |
| 2-639 | 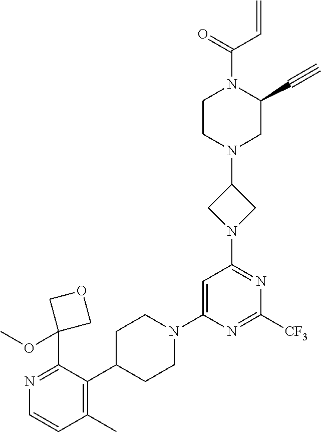 [SMILES: C#C[C@H]1CN(C2CN(c3cc(N4CCC(c5c(C)ccnc5C5(OC)COC5)CC4)nc(C(F)(F)F)n3)C2)CCN1C(=O)C=C] | 1-((2S)-2-ethynyl-4-(1- (6-(4-(2-(3-methoxy-3- oxetanyl)-4-methyl-3- pyridinyl)-1-piperidinyl)- 2-(trifluoromethyl)-4- pyrimidinyl)-3-azetidin- yl)-1-piperazinyl)-2- propen-1-one | E-12 | H-46-2 | 626.4 |
| 2-641 | 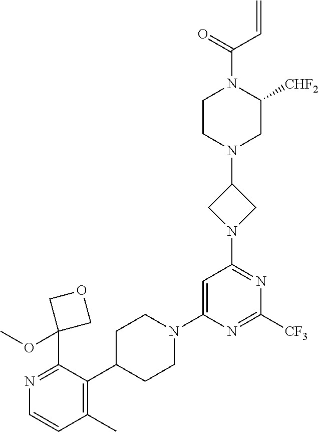 [SMILES: C=CC(=O)N1CCN(C2CN(c3cc(N4CCC(c5c(C)ccnc5C5(OC)COC5)CC4)nc(C(F)(F)F)n3)C2)C[C@H]1C(F)F] | 1-((2S)-2-(difluorometh- yl)-4-(1-(6-(4-(2-3-meth- oxy-3-oxetanyl)-4-meth- yl-3-pyridinyl)-1-piperi- dinyl)-2-(trifluorometh- yl)-4-pyrimidinyl)-3- azetidinyl)-1-piperazin- yl)-2-propen-1-one | E-7 | H-47-1 | 652.2 |

### SFC Conditions for Chiral Separation

| Int. # | Chemical Structure | Name | Separation condition |
|---|---|---|---|
| H-1-1 | 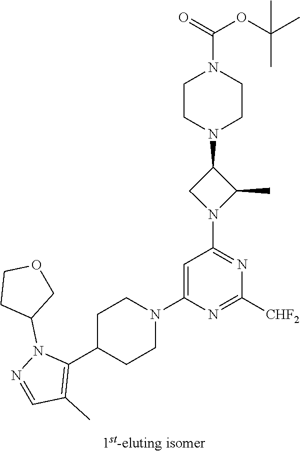 [SMILES: Cc1cnn(C2CCOC2)c1C1CCN(c2cc(N3C[C@@H](N4CCN(C(=O)OC(C)(C)C)CC4)[C@H]3C)nc(C(F)F)n2)CC1] | tert-butyl 4-((2R,3R)-2- methyl-1-(6-((1R,6R)-5-(4- methyl-1-(oxetan-3-yl)-1H- pyrazol-5-yl)-2- azabicyclo[4.1.0]heptan-2- yl)-2- (trifluoromethyl)pyrimidin-4- yl)azetidin-3-yl)piperazine-1- carboxylate | Chiralcel OD-H, 21 × 250 mm 5 μm, column with a mobile phase of 8:2 CO₂:IPA using a flow rate of 100 mL/min |
| H-2-2 | 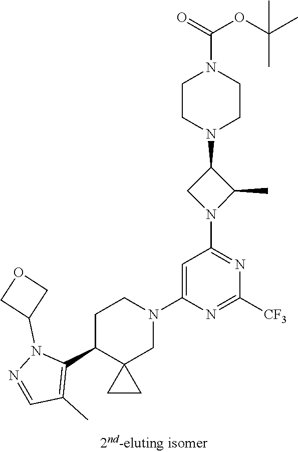 [SMILES: Cc1cnn(C2COC2)c1[C@H]1CCN(c2cc(N3C[C@@H](N4CCN(C(=O)OC(C)(C)C)CC4)[C@H]3C)nc(C(F)(F)F)n2)CC12CC2] | 1-(4-((2R,3R)-2-methyl-1-(6- ((S)-8-(4-methyl-1-(oxetan-3- yl)-1H-pyrazol-5-yl)-5- azaspiro[2.5]octan-5-yl)-2- (trifluoromethyl)pyrimidin-4- yl)azetidin-3-yl)piperazin-1- yl)prop-2-en-1-one | LUXC4, 250 × 50 mm 5 μm, column with a mobile phase of 15% ACN and 15% MeOH in liquid CO₂ using a flow rate of 180 mL/min |
| H-3-1 | 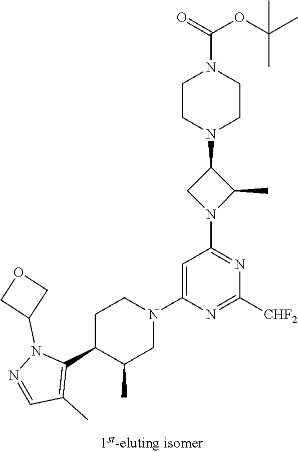 [SMILES: Cc1cnn(C2COC2)c1[C@H]1CCN(c2cc(N3C[C@@H](N4CCN(C(=O)OC(C)(C)C)CC4)[C@H]3C)nc(C(F)F)n2)C[C@H]1C] | tert-butyl 4-((2R,3R)-1-(2- (difluoromethyl)-6-((3S,4S)- 3-methyl-4-(4-methyl-1- (oxetan-3-yl)-1H-pyrazol-5- yl)piperidin-1-yl)pyrimidin-4- yl)-2-methylazetidin-3- yl)piperazine-1-carboxylate | Chiralpak OJ-H, 30 × 250 mm 5 μm, column with a mobile phase of 8:2 CO₂: [ACN:MeOH (1:1)] using a flow rate of 80 mL/min |
| H-3-2 | 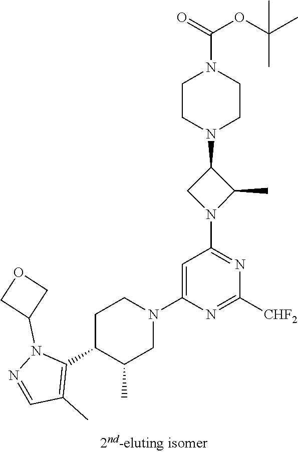 [SMILES: Cc1cnn(C2COC2)c1[C@@H]1CCN(c2cc(N3C[C@@H](N4CCN(C(=O)OC(C)(C)C)CC4)[C@H]3C)nc(C(F)F)n2)C[C@@H]1C] | tert-butyl 4-((2R,3R)-1-(2- (difluoromethyl)-6-((3R,4R)- 3-methyl-4-(4-methyl-1- (oxetan-3-yl)-1H-pyrazol-5- yl)piperidin-1-yl)pyrimidin-4- yl)-2-methylazetidin-3- yl)piperazine-1-carboxylate | Chiralpak OJ-H, 30 × 250 mm 5 μm, column with a mobile phase of 8:2 CO₂: [ACN:MeOH (1:1)] using a flow rate of 80 mL/min |
| H-4-1 | 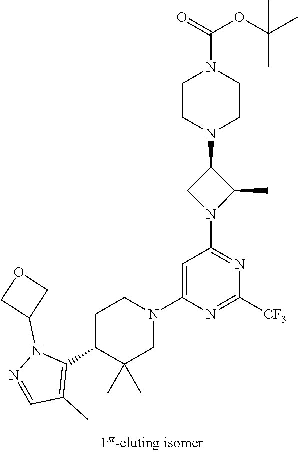 [SMILES: Cc1cnn(C2COC2)c1[C@@H]1CCN(c2cc(N3C[C@@H](N4CCN(C(=O)OC(C)(C)C)CC4)[C@H]3C)nc(C(F)(F)F)n2)CC1(C)C] | tert-butyl 4-((2R,3R)-1-(6- ((R)-3,3-dimethyl-4-(4- methyl-1-(oxetan-3-yl)-1H- pyrazol-5-yl)piperidin-1-yl)- 2-(trifluoromethyl)pyrimidin- 4-yl)-2-methylazetidin-3- yl)piperazine-1-carboxylate | LUXC4, 250 × 50 mm 5 μm, column with a mobile phase of 40% EtOH in liquid CO₂ using a flow rate of 170 mL/min |
| H-4-2 | 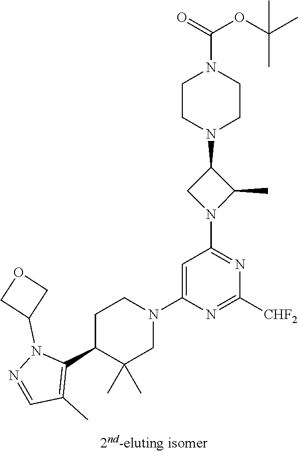 [SMILES: Cc1cnn(C2COC2)c1[C@H]1CCN(c2cc(N3C[C@@H](N4CCN(C(=O)OC(C)(C)C)CC4)[C@H]3C)nc(C(F)F)n2)CC1(C)C] | tert-butyl 4-((2R,3R)-1-(6- ((S)-3,3-dimethyl-4-(4- methyl-1-(oxetan-3-yl)-1H- pyrazol-5-yl)piperidin-1-yl)- 2-(trifluoromethyl)pyrimidin- 4-yl)-2-methylazetidin-3- yl)piperazine-1-carboxylate | LUXC4, 250 × 50 mm 5 μm, column with a mobile phase of 40% EtOH in liquid CO₂ using a flow rate of 170 mL/min |
| H-5-1 | 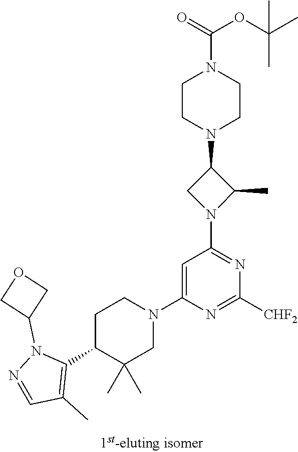 [SMILES: Cc1cnn(C2COC2)c1[C@@H]1CCN(c2cc(N3C[C@@H](N4CCN(C(=O)OC(C)(C)C)CC4)[C@H]3C)nc(C(F)F)n2)CC1(C)C] | tert-butyl 4-((2R,3R)-1-(2- (difluoromethyl)-6-((R)-3,3- dimethyl-4-(4-methyl-1- (oxetan-3-yl)-1H-pyrazol-5- yl)piperidin-1-yl)pyrimidin-4- yl)-2-methylazetidin-3- yl)piperazine-1-carboxylate | Chiralpak IG, 250 × 50 mm 5 μm, column with a mobile phase of 30% MeOH in liquid CO₂ using a flow rate of 120 mL/min |
| H-5-2 | 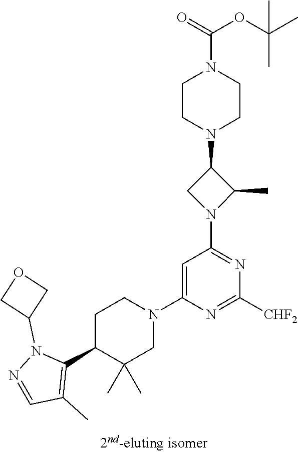 [SMILES: Cc1cnn(C2COC2)c1[C@H]1CCN(c2cc(N3C[C@@H](N4CCN(C(=O)OC(C)(C)C)CC4)[C@H]3C)nc(C(F)F)n2)CC1(C)C] | tert-butyl 4-((2R,3R)-1-(2- (difluoromethyl)-6-((S)-3,3- dimethyl-4-(4-methyl-1- (oxetan-3-yl)-1H-pyrazol-5- yl)piperidin-1-yl)pyrimidin-4- yl)-2-methylazetidin-3- yl)piperazine-1-carboxylate | Chiralpak IG, 250 × 50 mm 5 μm, column with a mobile phase of 30% MeOH in liquid CO₂ using a flow rate of 120 mL/min |
| H-6-1 | 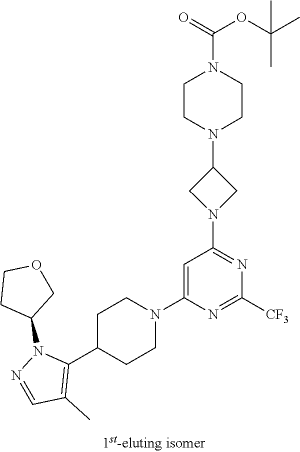 [SMILES: Cc1cnn([C@H]2CCOC2)c1C1CCN(c2cc(N3CC(N4CCN(C(=O)OC(C)(C)C)CC4)C3)nc(C(F)(F)F)n2)CC1] | tert-butyl (S)-4-(1-(6-(4-(4- methyl-1-(tetrahydrofuran-3- yl)-1H-pyrazol-5- yl)piperidin-1-yl)-2- (trifluoromethyl)pyrimidin-4- yl)azetidin-3- yl)piperazine-1-carboxylate | Chiralpak IC, 250 × 21 mm 5 μm, column with a mobile phase of 40% MeOH in liquid CO₂ using a flow rate of 90 mL/min |
| H-6-2 | 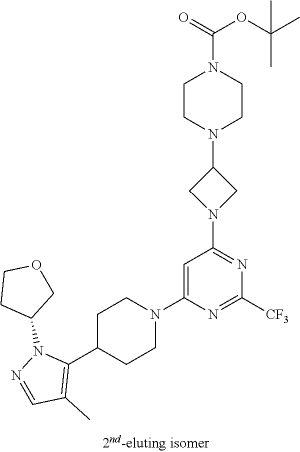 [SMILES: Cc1cnn([C@@H]2CCOC2)c1C1CCN(c2cc(N3CC(N4CCN(C(=O)OC(C)(C)C)CC4)C3)nc(C(F)(F)F)n2)CC1] | tert-butyl (R)-4-(1-(6-(4-(4- methyl-1-(tetrahydrofuran-3- yl)-1H-pyrazol-5- yl)piperidin-1-yl)-2- (trifluoromethyl)pyrimidin-4- yl)azetidin-3- yl)piperazine-1-carboxylate | Chiralpak IC, 250 × 21 mm 5 μm, column with a mobile phase of 40% MeOH in liquid CO₂ using a flow rate of 90 mL/min |
| H-7-1 | 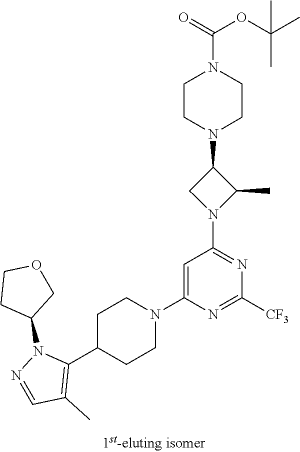 [SMILES: Cc1cnn([C@H]2CCOC2)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-2- methyl-1-(6-(4-(4-methyl-1- ((S)-tetrahydrofuran-3-yl)- 1H-pyrazol-5-yl)piperidin-1- yl)-2- (trifluoromethyl)pyrimidin-4- yl)azetidin-3- yl)piperazine-1-carboxylate | Chiralcel ODH, 250 × 30 mm 5 μm, column with a mobile phase of 20% IPA in liquid CO₂ using a flow rate of 120 mL/min |
| H-7-2 | 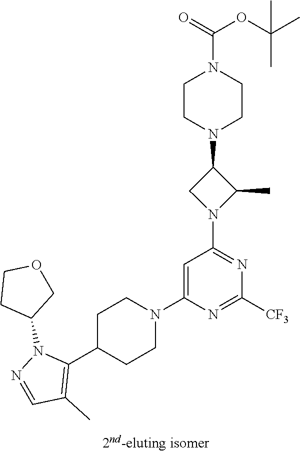 [SMILES: Cc1cnn([C@@H]2CCOC2)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-2- methyl-1-(6-(4-(4-methyl-1- ((R)-tetrahydrofuran-3-yl)- 1H-pyrazol-5-yl)piperidin-1- yl)-2- (trifluoromethyl)pyrimidin-4- yl)azetidin-3- yl)piperazine-1-carboxylate | Chiralcel ODH, 250 × 30 mm 5 μm, column with a mobile phase of 20% IPA in liquid CO₂ using a flow rate of 120 mL/min |
| H-8-1 | 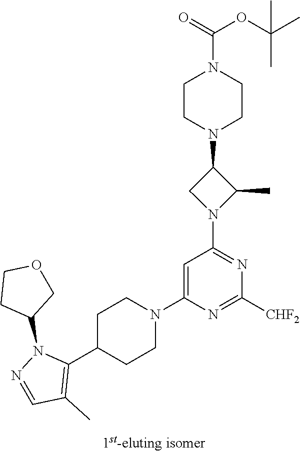 | tert-butyl 4-((2R,3R)-1-(2- (difluoromethyl)-6-(4-(4- methyl-1-((S)- tetrahydrofuran-3-yl)-1H- pyrazol-5-yl)piperidin-1- yl)pyrimidin-4-yl)-2- methylazetidin-3- yl)piperazine-1-carboxylate | Chiralcel OD-H, 250 × 21 mm 5 μm, column with a mobile phase of 20% IPA in liquid CO₂ using a flow rate of 120 mL/min |
| H-8-2 | 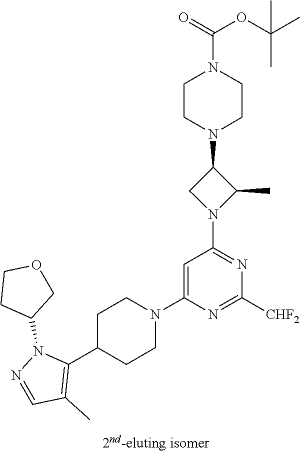 | tert-butyl 4-((2R,3R)-1-(2- (difluoromethyl)-6-(4-(4- methyl-1-((R)- tetrahydrofuran-3-yl)-1H- pyrazol-5-yl)piperidin-1- yl)pyrimidin-4-yl)-2- methylazetidin-3- yl)piperazine-1-carboxylate | Chiralcel OD-H, 250 × 21 mm 5 μm, column with a mobile phase of 20% IPA in liquid CO₂ using a flow rate of 120 mL/min |
| H-9-1 | 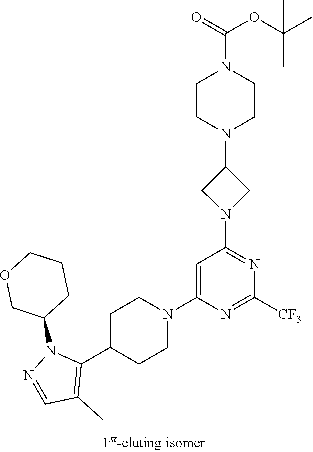 [SMILES: Cc1cnn([C@@H]2CCCOC2)c1C1CCN(c2cc(N3CC(N4CCN(C(=O)OC(C)(C)C)CC4)C3)nc(C(F)(F)F)n2)CC1] | tert-butyl (R)-4-(1-(6-(4-(4- methyl-1-(tetrahydro-2H- pyran-3-yl)-1H-pyrazol-5- yl)piperidin-1-yl)-2- (trifluoromethyl)pyrimidin-4- yl)azetidin-3-yl)piperazine-1- carboxylate | Chiralpak IC, 250 × 50 mm 5 μm, column with a mobile phase of 20% MeOH and 20% ACN in liquid CO₂ using a flow rate of 120 mL/min) |
| H-9-2 | 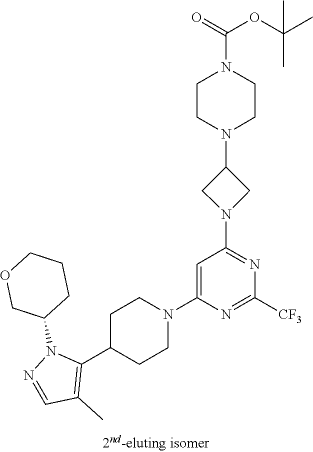 [SMILES: Cc1cnn([C@H]2CCCOC2)c1C1CCN(c2cc(N3CC(N4CCN(C(=O)OC(C)(C)C)CC4)C3)nc(C(F)(F)F)n2)CC1] | tert-butyl (S)-4-(1-(6-(4-(4- methyl-1-(tetrahydro-2H- pyran-3-yl)-1H-pyrazol-5- yl)piperidin-1-yl)-2- (trifluoromethyl)pyrimidin-4- yl)azetidin-3- yl)piperazine-1-carboxylate | Chiralpak IC, 100 × 50 mm 5 μm, column with a mobile phase of 15% MeOH and 15% ACN in liquid CO₂ using a flow rate of 120 mL/min) |
| H-10-1 | 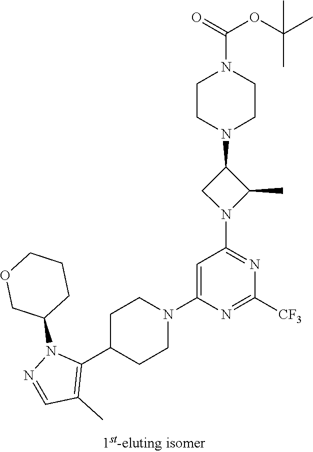 [SMILES: Cc1cnn([C@@H]2CCCOC2)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-2- methyl-1-(6-(4-(4-methyl-1- ((R)-tetrahydro-2H-pyran-3- yl)-1H-pyrazol-5- yl)piperidin-1-yl)-2- (trifluoromethyl)pyrimidin- 4-yl)azetidin-3- yl)piperazine-1-carboxylate | Chiralpak AD-H, 100 × 50 mm 5 μm, column with a mobile phase of 30% EtOH in liquid CO₂ using a flow rate of 100 mL/min |
| H-10-2 | 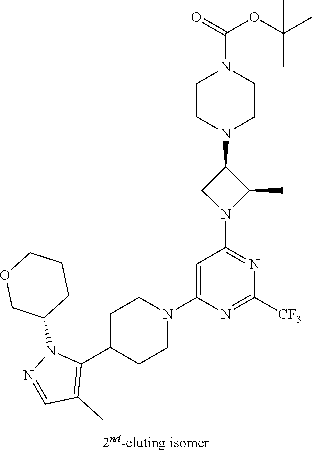 [SMILES: Cc1cnn([C@H]2CCCOC2)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-2- methyl-1-(6-(4-(4-methyl-1- ((S)-tetrahydro-2H-pyran-3- yl)-1H-pyrazol-5- yl)piperidin-1-yl)-2- (trifluoromethyl)pyrimidin-4- yl)azetidin-3- yl)piperazine-1-carboxylate | Chiralpak AD-H, 100 × 50 mm 5 μm, column with a mobile phase of 30% EtOH in liquid CO₂ using a flow rate of 100 mL/min |
| H-11-1 | 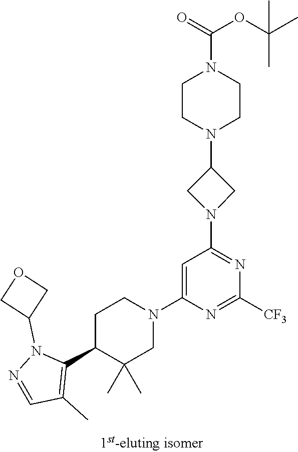 [SMILES: Cc1cnn(C2COC2)c1[C@H]1CCN(c2cc(N3CC(N4CCN(C(=O)OC(C)(C)C)CC4)C3)nc(C(F)(F)F)n2)CC1(C)C] | tert-butyl (S)-4-(1-(6-(3,3- dimethyl-4-(4-methyl-1- (oxetan-3-yl)-1H-pyrazol-5- yl)piperidin-1-yl)-2- (trifluoromethyl)pyrimidin-4- yl)azetidin-3- yl)piperazine-1-carboxylate | LUXC4, 250 × 50 mm 5 μm, column with a mobile phase of 40% MeOH in liquid CO₂ using a flow rate of 180 mL/min |
| H-11-2 | 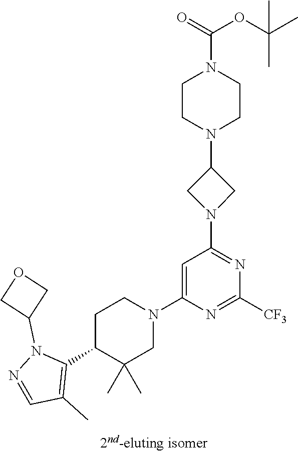 [SMILES: Cc1cnn(C2COC2)c1[C@@H]1CCN(c2cc(N3CC(N4CCN(C(=O)OC(C)(C)C)CC4)C3)nc(C(F)(F)F)n2)CC1(C)C] | tert-butyl (R)-4-(1-(6-(3,3- dimethyl-4-(4-methyl-1- (oxetan-3-yl)-1H-pyrazol-5- yl)piperidin-1-yl)-2- (trifluoromethyl)pyrimidin-4- yl)azetidin-3- yl)piperazine-1-carboxylate | LUXC4, 250 × 50 mm 5 μm, column with a mobile phase of 40% MeOH in liquid CO₂ using a flow rate of 180 mL/min |
| H-12-1 | 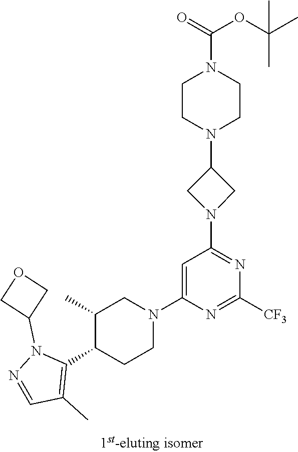 [SMILES: Cc1cnn(C2COC2)c1[C@H]1CCN(c2cc(N3CC(N4CCN(C(=O)OC(C)(C)C)CC4)C3)nc(C(F)(F)F)n2)C[C@H]1C] | tert-butyl 4-(1-(6-((3S,4S)-3- methyl-4-(4-methyl-1- (oxetan-3-yl)-1H-pyrazol-5- yl)piperidin-1-yl)-2- (trifluoromethyl)pyrimidin-4- yl)azetidin-3- yl)piperazine-1-carboxylate | Chiralpak OJH, 250 × 50 mm 5 μm, column with a mobile phase of 20% MeOH in liquid CO₂ using a flow rate of 150 mL/min |
| H-13-1 | 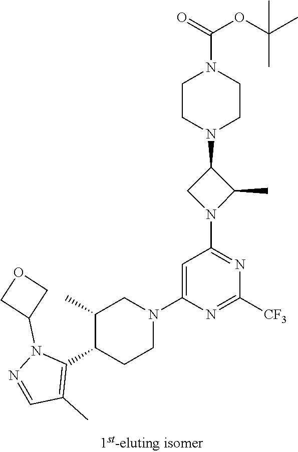 [SMILES: Cc1cnn(C2COC2)c1[C@H]1CCN(c2cc(N3C[C@@H](N4CCN(C(=O)OC(C)(C)C)CC4)[C@H]3C)nc(C(F)(F)F)n2)C[C@H]1C] | tert-butyl 4-((2R,3R)-2- methyl-1-(6-((3S,4S)-3- methyl-4-(4-methyl-1- (oxetan-3-yl)-1H-pyrazol-5- yl)piperidin-1-yl)-2- (trifluoromethyl)pyrimidin- 4-yl)azetidin-3- yl)piperazine-1-carboxylate | Chiralcel OJH, 250 × 30 mm 5 μm, column with a mobile phase of 20% MeOH in liquid CO₂ using a flow rate of 90 mL/min |
| H-14-1 | 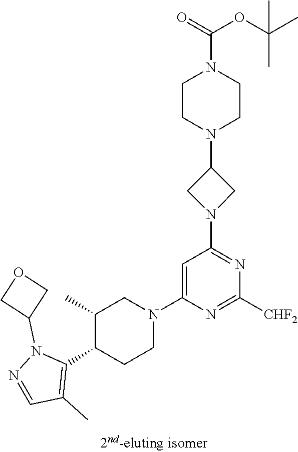 [SMILES: Cc1cnn(C2COC2)c1[C@H]1CCN(c2cc(N3CC(N4CCN(C(=O)OC(C)(C)C)CC4)C3)nc(C(F)F)n2)C[C@H]1C] | tert-butyl 4-((2R,3R)-1-(2- (difluoromethyl)-6-((3S,4S)- 3-methyl-4-(4-methyl-1- (oxetan-3-yl)-1H-pyrazol-5- yl)piperidin-1-yl)pyrimidin-4- yl)-2-methylazetidin-3- yl)piperazine-1-carboxylate | Chiralpak IG, 250 × 30 mm 5 μm, column with a mobile phase of 50% MeOH in liquid CO₂ using a flow rate of 180 mL/min |
| H-15-1 | 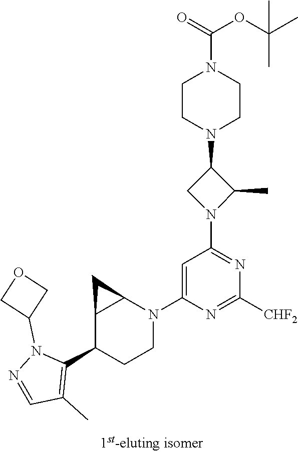 [SMILES: Cc1cnn(C2COC2)c1[C@@H]1CCN(c2cc(N3C[C@@H](N4CCN(C(=O)OC(C)(C)C)CC4)[C@H]3C)nc(C(F)F)n2)[C@@H]2C[C@@H]21] | tert-butyl 4-((2R,3R)-1-(2- (difluoromethyl)-6- ((1R,5R,6R)-5-(4-methyl-1- (oxetan-3-yl)-1H-pyrazol-5- yl)-2- azabicyclo[4.1.0]heptan-2- yl)pyrimidin-4-yl)-2- methylazetidin-3- yl)piperazine-1-carboxylate | Chiralcel ODH, 250 × 30 mm 5 μm, column with a mobile phase of 15% IPA in liquid CO₂ using a flow rate of 120 mL/min |
| H-16-1 | 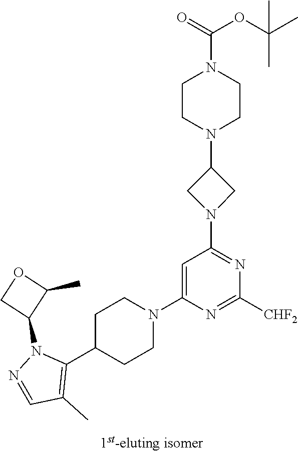 [SMILES: Cc1cnn([C@H]2CO[C@H]2C)c1C1CCN(c2cc(N3CC(N4CCN(C(=O)OC(C)(C)C)CC4)C3)nc(C(F)F)n2)CC1] | tert-butyl 4-(1-(2- (difluoromethyl)-6-(4-(4- methyl-1-((2S,3S)-2- methyloxetan-3-yl)-1H- pyrazol-5-yl)piperidin-1- yl)pyrimidin-4-yl)azetidin-3- yl)piperazine-1-carboxylate | Chiralpak IC, 250 × 21 mm 5 μm, column with a mobile phase of 15% MeOH and 15% ACN in liquid CO₂ using a flow rate of 120 mL/min |
| H-16-2 | 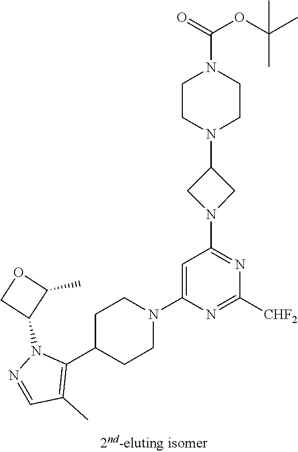 [SMILES: Cc1cnn([C@@H]2CO[C@@H]2C)c1C1CCN(c2cc(N3CC(N4CCN(C(=O)OC(C)(C)C)CC4)C3)nc(C(F)F)n2)CC1] | tert-butyl 4-(1-(2- (difluoromethyl)-6-(4-(4- methyl-1-((2R,3R)-2- methyloxetan-3-yl)-1H- pyrazol-5-yl)piperidin-1- yl)pyrimidin-4-yl)azetidin-3- yl)piperazine-1-carboxylate | Chiralpak IC, 250 × 21 mm 5 μm, column with a mobile phase of 15% MeOH and 15% ACN in liquid CO₂ using a flow rate of 120 mL/min |
| H-17-1 | 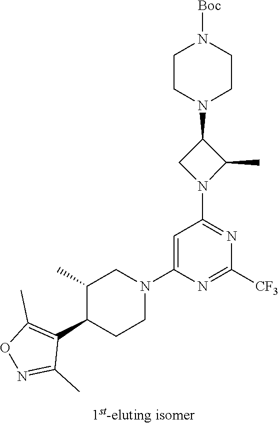 [SMILES: Cc1noc(C)c1[C@@H]1CCN(c2cc(N3C[C@@H](N4CCN(C(=O)OC(C)(C)C)CC4)[C@H]3C)nc(C(F)(F)F)n2)C[C@H]1C] | tert-butyl 4-((2R,3R)-1-(6- ((3S,4R)-4-(3,5- dimethylisoxazol-4-yl)-3- methylpiperidin-1-yl)-2- (trifluoromethyl)pyrimidin-4- yl)-2-methylazetidin-3- yl)piperazine-1-carboxylate | Lux Cellulose-2, 2 × 25 cm 5 μm column with a mobile phase of 25% MeOH using a flow rate of 80 mL/min. The combined 1^{st} & 2^{nd} eluting peaks were further purified using a Chiralcel OX, 2 × 25 cm 5 μm column with a mobile phase of 40% iPrOH using a flow rate of 100 mL/min |
| H-17-2 | 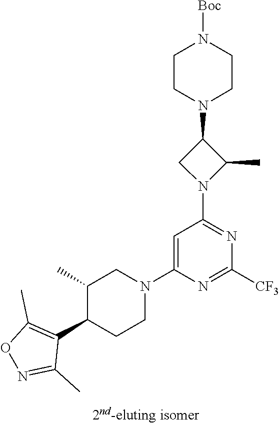 [SMILES: Cc1noc(C)c1[C@@H]1CCN(c2cc(N3C[C@@H](N4CCN(C(=O)OC(C)(C)C)CC4)[C@H]3C)nc(C(F)(F)F)n2)C[C@H]1C] | tert-butyl 4-((2R,3R)-1-(6- ((3R,4S)-4-(3,5- dimethylisoxazol-4-yl)-3- methylpiperidin-1-yl)-2- (trifluoromethyl)pyrimidin-4- yl)-2-methylazetidin-3- yl)piperazine-1-carboxylate | Lux Cellulose-2, 2 × 25 cm 5 μm column with a mobile phase of 25% MeOH using a flow rate of 80 mL/min. The combined 1^{st} & 2^{nd} eluting peaks were further purified using a Chiralcel OX, 2 × 25 cm 5 μm column with a mobile phase of 40% iPrOH using a flow rate of 100 mL/min |
| H-17-3 | 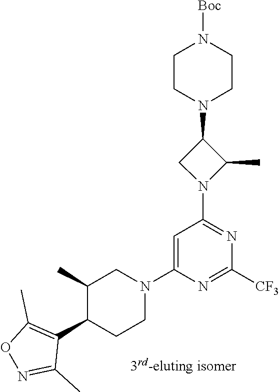 [SMILES: Cc1noc(C)c1[C@@H]1CCN(c2cc(N3C[C@@H](N4CCN(C(=O)OC(C)(C)C)CC4)[C@H]3C)nc(C(F)(F)F)n2)C[C@@H]1C] | tert-butyl 4-((2R,3R)-1-(6- ((3R,4R)-4-(3,5- dimethylisoxazol-4-yl)-3- methylpiperidin-1-yl)-2- (trifluoromethyl)pyrimidin-4- yl)-2-methylazetidin-3- yl)piperazine-1-carboxylate | Lux Cellulose-2, 2 × 25 cm 5 μm column with a mobile phase of 25% MeOH using a flow rate of 80 mL/min. The combined 3rd & 4^{th} eluting peaks were further purified using a Chiralcel IC, 2 × 25 cm 5 μm column with a mobile phase of 40% MeOH using a flow rate of 100 mL/min |
| H-18-1 | 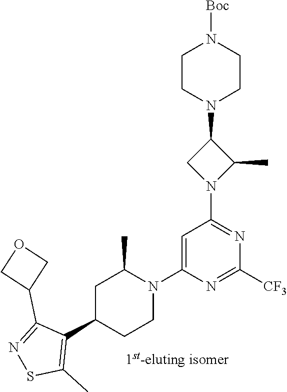 [SMILES: Cc1snc(C2COC2)c1[C@@H]1CCN(c2cc(N3C[C@@H](N4CCN(C(=O)OC(C)(C)C)CC4)[C@H]3C)nc(C(F)(F)F)n2)[C@H](C)C1] | tert-butyl 4-((2R,3R)-2- methyl-1-(6-((2R,4R)-2- methyl-4-(5-methyl-3- (oxetan-3-yl)isothiazol-4- yl)piperidin-1-yl)-2- (trifluoromethyl)pyrimidin-4- yl)azetidin-3- yl)piperazine-1-carboxylate | Chiralcel OX, 2 × 25 cm 5 μm column with a mobile phase of 40%, MeOH using a flow rate of 80 mL/min |
| H-18-2 | 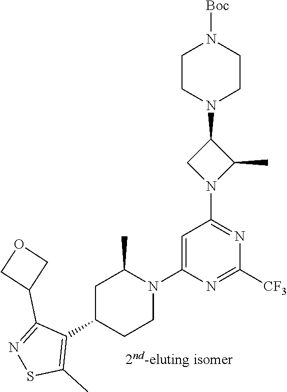 [SMILES: Cc1snc(C2COC2)c1[C@H]1CCN(c2cc(N3C[C@@H](N4CCN(C(=O)OC(C)(C)C)CC4)[C@H]3C)nc(C(F)(F)F)n2)[C@H](C)C1] | tert-butyl 4-((2R,3R)-2- methyl-1-(6-((2R,4S)-2- methyl-4-(5-methyl-3- (oxetan-3-yl)isothiazol-4- yl)piperidin-1-yl)-2- (trifluoromethyl)pyrimidin-4- yl)azetidin-3- yl)piperazine-1-carboxylate | Chiralcel OX, 2 × 25 cm 5 μm column with a mobile phase of 40% MeOH using a flow rate fo 80 mL/min |
| H-19-1 | 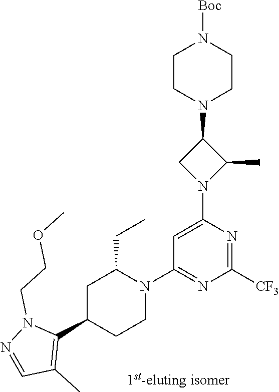 [SMILES: CC[C@H]1C[C@H](c2c(C)cnn2CCOC)CCN1c1cc(N2C[C@@H](N3CCN(C(=O)OC(C)(C)C)CC3)[C@H]2C)nc(C(F)(F)F)n1] | tert-butyl 4-((2R,3R)-1-(6- ((2S,4R)-2-ethyl-4-(1-(2- methoxyethyl)-4-methyl-1H- pyrazol-5-yl)piperidin-1-yl)- 2-(trifluoromethyl)pyrimidin- 4-yl)-2-methylazetidin-3- yl)piperazine-1-carboxylate | Chiralcel OX, 21 × 250 mm 5 μm, column with a mobile phase of 40% IPA using a flow rate of 150 mL/min |
| H-19-2 | 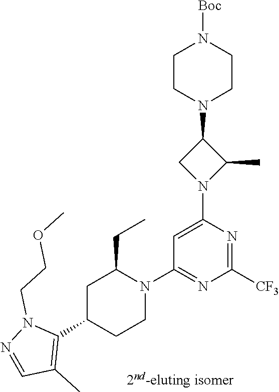 [SMILES: CC[C@@H]1C[C@@H](c2c(C)cnn2CCOC)CCN1c1cc(N2C[C@@H](N3CCN(C(=O)OC(C)(C)C)CC3)[C@H]2C)nc(C(F)(F)F)n1] | tert-butyl 4-((2R,3R)-1-(6- ((2R,4S)-2-ethyl-4-(1-(2- methoxyethyl)-4-methyl-1H- pyrazol-5-yl)piperidin-1-yl)- 2-(trifluoromethyl)pyrimidin- 4-yl)-2-methylazetidin-3- yl)piperazine-1-carboxylate | Chiralcel OX, 21 × 250 mm 5 μm, column with a mobile phase or 40% IPA using a flow rate of 150 mL/min |
| H-20-1 | 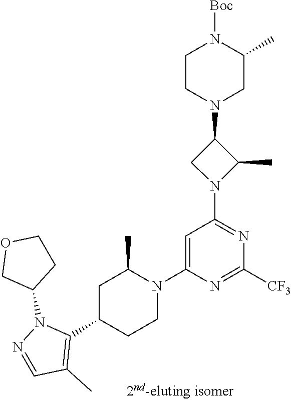 [SMILES: Cc1cnn([C@H]2CCOC2)c1[C@H]1CCN(c2cc(N3C[C@@H](N4CCN(C(=O)OC(C)(C)C)[C@H](C)C4)[C@H]3C)nc(C(F)(F)F)n2)[C@H](C)C1] | tert-butyl (R)-2-methyl-4- ((2R,3R)-2-methyl-1-(6- ((2R,4S)-2-methyl-4-(4- methyl-1-((S)- tetrahydrofuran-3-yl)-1H- pyrazol-5-yl)piperidin-1-yl)- 2-(trifluoromethyl)pyrimidin- 4-yl)azetidin-3- yl)piperazine-1-carboxylate | Chiralpak IG 2 × 15 cm, 5 μm column with a mobile phase of 25% MeOH using a flow rate of 80 mL/min |
| H-21-1 | 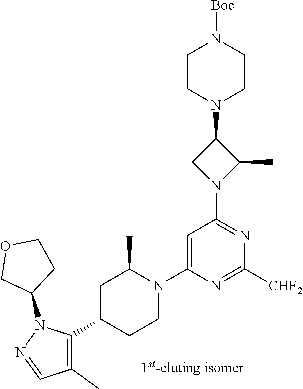 [SMILES: Cc1cnn([C@@H]2CCOC2)c1[C@H]1CCN(c2cc(N3C[C@@H](N4CCN(C(=O)OC(C)(C)C)CC4)[C@H]3C)nc(C(F)F)n2)[C@H](C)C1] | tert-butyl 4-((2R,3R)-1-(2- (difluoromethyl)-6-((2R,4S)- 2-methyl-4-(4-methyl-1-((R)- tetrahydrofuran-3-yl)-1H- pyrazol-5-yl)piperidin-1- yl)pyrimidin-4-yl)-2- methylazetidin-3- yl)piperazine-1-carboxylate | Chiralpak IG 2 × 25 cm, 5 μm column with a mobile phase of 35% MeOH using a flow rate of 80 mL/min |
| H-21-2 | 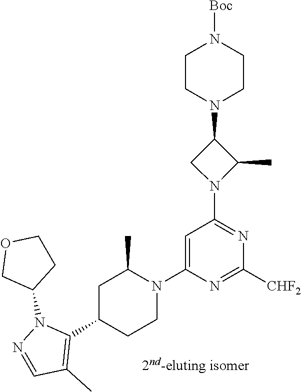 [SMILES: Cc1cnn([C@H]2CCOC2)c1[C@H]1CCN(c2cc(N3C[C@@H](N4CCN(C(=O)OC(C)(C)C)CC4)[C@H]3C)nc(C(F)F)n2)[C@H](C)C1] | tert-butyl 4-((2R,3R)-1-(2- (difluoromethyl)-6-((2R,4S)- 2-methyl-4-(4-methyl-1-((S)- tetrahydrofuran-3-yl)-1H- pyrazol-5-yl)piperidin-1- yl)pyrimidin-4-yl)-2- methylazetidin-3- yl)piperazine-1-carboxylate | Chiralpak IF 2 × 25 cm, 5 μm column with a mobile phase of 35% MeOH using a flow rate of 80 mL/min |
| H-22-1 | 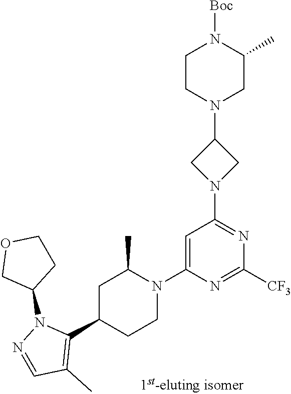 [SMILES: Cc1cnn([C@@H]2CCOC2)c1[C@@H]1CCN(c2cc(N3CC(N4CCN(C(=O)OC(C)(C)C)[C@H](C)C4)C3)nc(C(F)(F)F)n2)[C@H](C)C1] | tert-butyl (R)-2-methyl-4-(1- (6-((2R,4R)-2-methyl-4-(4- methyl-1-((R)- tetrahydrofuran-3-yl)-1H- pyrazol-5-yl)piperidin-1-yl)- 2-(trifluoromethyl)pyrimidin- 4-yl)azetidin-3-yl)piperazine- 1-carboxylate | Chiralpak IG 2 × 15 cm, 5 μm column with a mobile phase of 25% MeOH using a flow rate of 80 mL/min |
| H-22-2 | 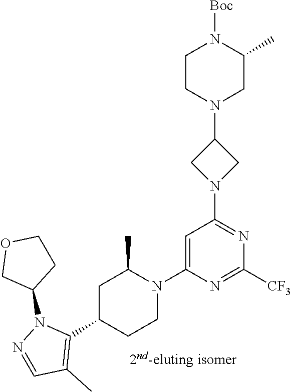 [SMILES: Cc1cnn([C@@H]2CCOC2)c1[C@H]1CCN(c2cc(N3CC(N4CCN(C(=O)OC(C)(C)C)[C@H](C)C4)C3)nc(C(F)(F)F)n2)[C@H](C)C1] | tert-butyl (R)-2-methyl-4-(1- (6-((2R,4S)-2-methyl-4-(4- methyl-1-((R)- tetrahydrofuran-3-yl)-1H- pyrazol-5-yl)piperidin-1-yl)- 2-(trifluoromethyl)pyrimidin- 4-yl)azetidin-3-yl)piperazine- 1-carboxylate | Chiralpak IC 2 × 15 cm, 5 μm column with a mobile phase of 45% MeOH using a flow rate of 55 mL/min |
| H-23-2 | 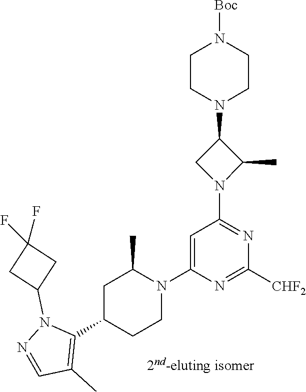 [SMILES: Cc1cnn(C2CC(F)(F)C2)c1[C@H]1CCN(c2cc(N3C[C@@H](N4CCN(C(=O)OC(C)(C)C)CC4)[C@H]3C)nc(C(F)F)n2)[C@H](C)C1] | tert-butyl 4-((2R,3R)-1-(6- ((2R,4S)-4-(1-(3,3- difluorocyclobutyl)-4-methyl- 1H-pyrazol-5-yl)-2- methylpiperidin-1-yl)-2- (difluoromethyl)pyrimidin-4- yl)-2-methylazetidin-3- yl)piperazine-1-carboxylate | Chiralpak IF 21 × 250 mm, 5 μm column with a mobile phase of 25% MeOH using a flow rate of 130 mL/min |
| H-24-2 | 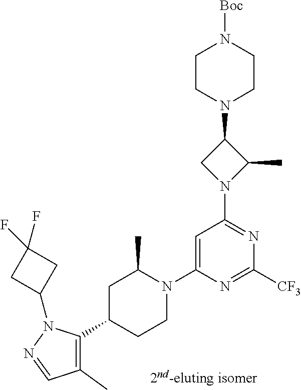 [SMILES: Cc1cnn(C2CC(F)(F)C2)c1[C@H]1CCN(c2cc(N3C[C@@H](N4CCN(C(=O)OC(C)(C)C)CC4)[C@H]3C)nc(C(F)(F)F)n2)[C@H](C)C1] | tert-butyl 4-((2R,3R)-1-(6- ((2R,4S)-4-(1-(3,3- difluorocyclobutyl)-4-methyl- 1H-pyrazol-5-yl)-2- methylpiperidin-1-yl)-2- (trifluoromethyl)pyrimidin-4- yl)-2-methylazetidin-3- yl)piperazine-1-carboxylate | Chiralpak IF 2 × 25 cm, 5 μm column with a mobile phase of 15% MeOH using a flow rate of 80 mL/min |
| H-25-4 | 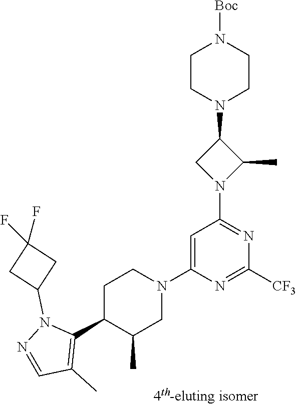 [SMILES: Cc1cnn(C2CC(F)(F)C2)c1[C@H]1CCN(c2cc(N3C[C@@H](N4CCN(C(=O)OC(C)(C)C)CC4)[C@H]3C)nc(C(F)(F)F)n2)C[C@H]1C] | tert-butyl 4-((2R,3R)-1-(6- ((3S,4S)-4-(1-(3,3- difluorocyclobutyl)-4-methyl- 1H-pyrazol-5-yl)-3- methylpiperidin-1-yl)-2- (trifluoromethyl)pyrimidin-4- yl)-2-methylazetidin-3- yl)piperazine-1-carboxylate | Chiralpak OD 21 × 250 mm, 5 μm column with a mobile phase of 10% MeOH using a flow rate of 80 mL/min |
| H-26-1 | 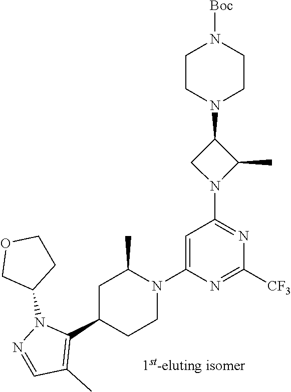 [SMILES: Cc1cnn([C@H]2CCOC2)c1[C@@H]1CCN(c2cc(N3C[C@@H](N4CCN(C(=O)OC(C)(C)C)CC4)[C@H]3C)nc(C(F)(F)F)n2)[C@H](C)C1] | tert-butyl 4-((2R,3R)-2- methyl-1-(6-((2R,4R)-2- methyl-4-(4-methyl-1-((S)- tetrahydrofuran-3-yl)-1H- pyrazol-5-yl)piperidin-1-yl)- 2-(trifluoromethyl)pyrimidin- 4-yl)azetidin-3- yl)piperazine-1-carboxylate | Chiralpak IF 30 × 250 mm, 5 μm column with a mobile phase of 25% MeOH using a flow rate of 200 mL/min |
| H-26-2 | 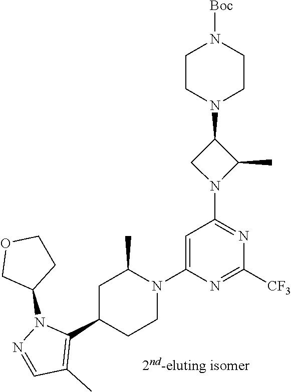 [SMILES: Cc1cnn([C@@H]2CCOC2)c1[C@@H]1CCN(c2cc(N3C[C@@H](N4CCN(C(=O)OC(C)(C)C)CC4)[C@H]3C)nc(C(F)(F)F)n2)[C@H](C)C1] | tert-butyl 4-((2R,3R)-2- methyl-1-(6-((2R,4R)-2- methyl-4-(4-methyl-1-((R)- tetrahydrofuran-3-yl)-1H- pyrazol-5-yl)piperidin-1-yl)- 2-(trifluoromethyl)pyrimidin- 4-yl)azetidin-3- yl)piperazine-1-carboxylate | Chiralpak IF 30 × 250 mm, 5 μm column with a mobile phase of 25% MeOH using a flow rate of 200 mL/min |
| H-27-3 | 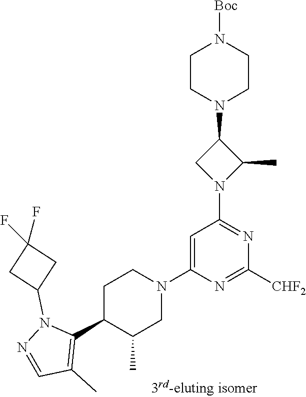 [SMILES: Cc1cnn(C2CC(F)(F)C2)c1[C@H]1CCN(c2cc(N3C[C@@H](N4CCN(C(=O)OC(C)(C)C)CC4)[C@H]3C)nc(C(F)F)n2)C[C@@H]1C] | tert-butyl 4-((2R,3R)-1-(6- ((3R,4S)-4-(1-(3,3- difluorocyclobutyl)-4-methyl- 1H-pyrazol-5-yl)-3- methylpiperidin-1-yl)-2- (difluoromethyl)pyrimidin-4- yl)-2-methylazetidin-3- yl)piperazine-1-carboxylate | Chiralpak OD 21 × 250 mm, 5 μm column with a mobile phase of 10% MeOH using a flow rate of 80 mL/min |
| H-28-1 | 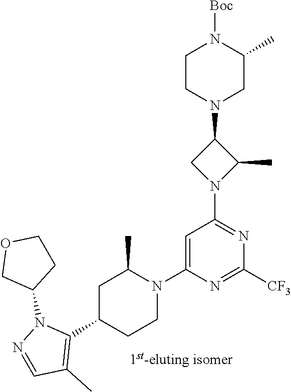 [SMILES: Cc1cnn([C@H]2CCOC2)c1[C@H]1CCN(c2cc(N3C[C@@H](N4CCN(C(=O)OC(C)(C)C)[C@H](C)C4)[C@H]3C)nc(C(F)(F)F)n2)[C@H](C)C1] | tert-butyl (R)-2-methyl-4- ((2R,3R)-2-methyl-1-(6- ((2R,4S)-2-methyl-4-(4- methyl-1-((S)- tetrahydrofuran-3-yl)-1H- pyrazol-5-yl)piperidin-1-yl)- 2-(trifluoromethyl)pyrimidin- 4-yl)azetidin-3- yl)piperazine-1-carboxylate | Chiralpak OD 21 × 250 mm, 5 μm column with a mobile phase of 20% MeOH using a flow rate of 80 mL/min |
| H-29-1 | 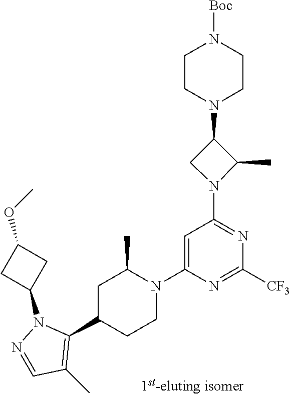 | tert-butyl 4-((2R,3R)-1-(6- ((2R,4R)-4-(1-((1r,3R)-3- methoxycyclobutyl)-4- methyl-1H-pyrazol-5-yl)-2- methylpiperidin-1-yl)-2- (trifluoromethyl)pyrimidin-4- yl)-2-methylazetidin-3- yl)piperazine-1-carboxylate | Chiralpak AD 2 × 25 cm 5 μm, column with a mobile phase of 15% EtOH using a flow rate of 100 mL/min |
| H-29-2 | 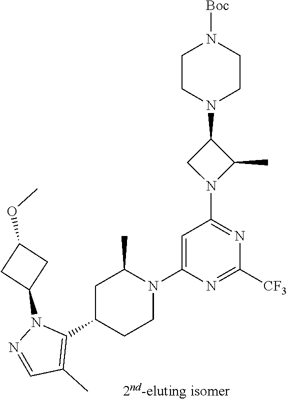 [SMILES: CO[C@H]1C[C@H](n2ncc(C)c2[C@H]2CCN(c3cc(N4C[C@@H](N5CCN(C(=O)OC(C)(C)C)CC5)[C@H]4C)nc(C(F)(F)F)n3)[C@H](C)C2)C1] | tert-butyl 4-((2R,3R)-1-(6- ((2R,4S)-4-(1-((1r,3S)-3- methoxycyclobutyl)-4- methyl-1H-pyrazol-5-yl)-2- methylpiperidin-1-yl)-2- (trifluoromethyl)pyrimidin-4- yl)-2-methylazetidin-3- yl)piperazine-1-carboxylate | Chiralpak AD 2 × 25 cm 5 μm, column with a mobile phase of 15% EtOH using a flow rate of 100 mL/min |
| H-30-1 | 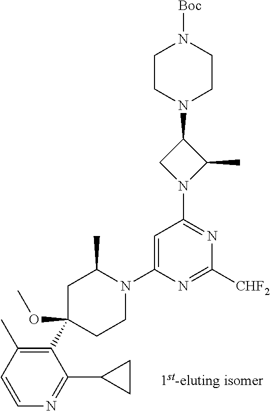 [SMILES: CO[C@]1(c2c(C)ccnc2C2CC2)CCN(c2cc(N3C[C@@H](N4CCN(C(=O)OC(C)(C)C)CC4)[C@H]3C)nc(C(F)F)n2)[C@H](C)C1] | tert-butyl 4-((2R,3R)-1-(6- ((2R,4R)-4-(2-cyclopropyl-4- methylpyridin-3-yl)-4- methoxy-2-methylpiperidin- 1-yl)-2- (difluoromethyl)pyrimidin-4- yl)-2-methylazetidin-3- yl)piperazine-1-carboxylate | Chiralpak AD, 21 × 150 mm 5 μm. column with a mobile phase of 25% EtOH using a flow rate of 150 mL/min |
| H-31-1 | 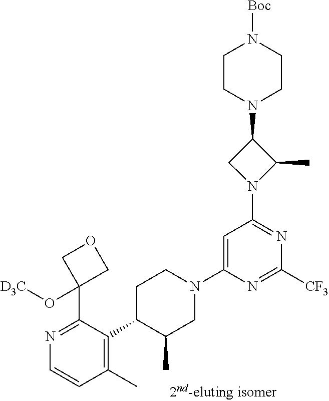 [SMILES: [2H]C([2H])([2H])OC1(c2nccc(C)c2[C@@H]2CCN(c3cc(N4C[C@@H](N5CCN(C(=O)OC(C)(C)C)CC5)[C@H]4C)nc(C(F)(F)F)n3)C[C@H]2C)COC1] | tert-butyl 4-((2R,3R)-1-(6- ((3S,4R)-4-(2-(3-(methoxy- d3)oxetan-3-yl)-4- methylpyridin-3-yl)-3- methylpiperidin-1-yl)-2- (trifluoromethyl)pyrimidin-4- yl)-2-methylazetidin-3- yl)piperazine-1-carboxylate | Chiralpak AD 21 × 150 mm 5 μm, column with a mobile phase of 10% EtOH using a flow rate of 200 mL/min |
| H-31-2 | 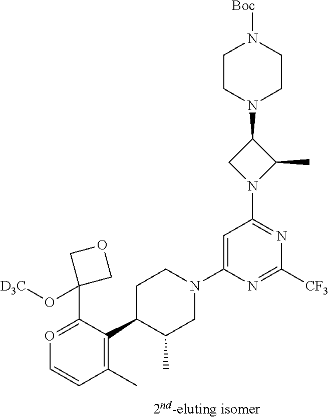 | tert-butyl 4-((2R,3R)-1-(6- ((3R,4S)-4-(2-(3-(methoxy- d3)oxetan-3-yl)-4- methylpyridin-3-yl)-3- methylpiperidin-1-yl)-2- (trifluoromethyl)pyrimidin-4- yl)-2-methylazetidin-3- yl)piperazine-1-carboxylate | Chiralpak AD 21 × 150 mm 5 μm, column with a mobile phase of 10% EtOH using a flow rate of 200 mL/min |
| H-32-1 | 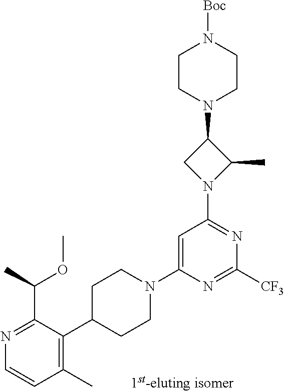 [SMILES: CO[C@H](C)c1nccc(C)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-1-(6-(4- (2-((R)-1-methoxyethyl)-4- methylpyridin-3-yl)piperidin- 1-yl)-2- (trifluoromethyl)pyrimidin-4- yl)-2-methylazetidin-3- yl)piperazine-1-carboxylate | Chiralcel OD-H 250 × 30 mm, 5 μm, column with a mobile phase of 30% IPA using a flow rate of 80 mL/min |
| H-32-2 | 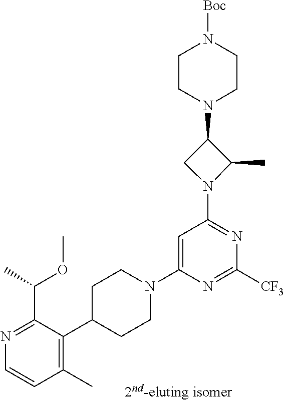 [SMILES: CO[C@@H](C)c1nccc(C)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-1-(6-(4- (2-((S)-1-methoxyethyl)-4- methylpyridin-3-yl)piperidin- 1-yl)-2- (trifluoromethyl)pyrimidin-4- yl)-2-methylazetidin-3- yl)piperazine-1-carboxylate | Chiralcel OD-H 250 × 30 mm, 5 μm, column with a mobile phase of 30% IPA using a flow rate of 80 mL/min |
| H-33-1 | 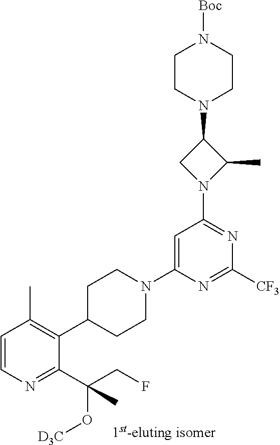 [SMILES: [2H]C([2H])([2H])O[C@@](C)(CF)c1nccc(C)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-1-(6-(4- (2-((R)-1-fluoro-2-(methoxy- d₃)propan-2-yl)-4- methylpyridin-3-yl)piperidin- 1-yl)-2- (trifluoromethyl)pyrimidin-4- yl)-2-methylazetidin-3- yl)piperazine-1-carboxylate | Chiralpak IC 150 × 46 mm 5 μm, column with a mobile phase of 30% MeOH using a flow rate of 180 mL/min |
| H-33-2 | 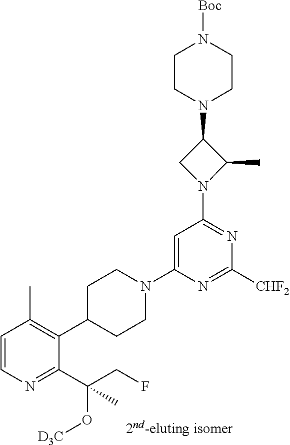 [SMILES: [2H]C([2H])([2H])O[C@](C)(CF)c1nccc(C)c1C1CCN(c2cc(N3C[C@@H](N4CCN(C(=O)OC(C)(C)C)CC4)[C@H]3C)nc(C(F)F)n2)CC1] | tert-butyl 4-((2R,3R)-1-(6-(4- (2-((S)-1-fluoro-2-(methoxy- d₃)propan-2-yl)-4- methylpyridin-3-yl)piperidin- 1-yl)-2- (trifluoromethyl)pyrimidin-4- yl)-2-methylazetidin-3- yl)piperazine-1-carboxylate | Chiralpak IC 150 × 46 mm 5 μm, column with a mobile phase of 30% MeOH using a flow rate of 180 mL/min |
| H-34-1 | 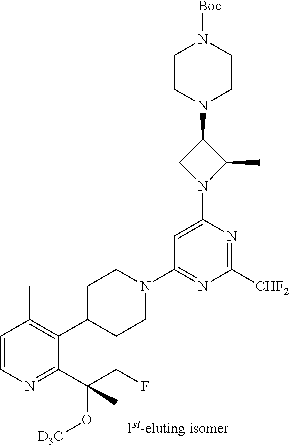 [SMILES: [2H]C([2H])([2H])O[C@@](C)(CF)c1nccc(C)c1C1CCN(c2cc(N3C[C@@H](N4CCN(C(=O)OC(C)(C)C)CC4)[C@H]3C)nc(C(F)F)n2)CC1] | tert-butyl 4-((2R,3R)-1-(2- (difluoromethyl)-6-(4-(2-((R)- 1-fluoro-2-(methoxy- d3)propan-2-yl)-4- methylpyridin-3-yl)piperidin- 1-yl)pyrimidin-4-yl)-2- methylazetidin-3- yl)piperazine-1-carboxylate | Chiralpak IC, 250 × 21 mm 5 μm, column with a mobile phase of 40% of (3:7) DCM:MeOH using a flow rate of 90 mL/min |
| H-34-2 | 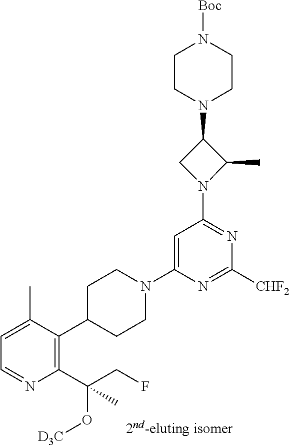 [SMILES: [2H]C([2H])([2H])O[C@](C)(CF)c1nccc(C)c1C1CCN(c2cc(N3C[C@@H](N4CCN(C(=O)OC(C)(C)C)CC4)[C@H]3C)nc(C(F)F)n2)CC1] | tert-butyl 4-((2R,3R)-1-(2- (difluoromethyl)-6-(4-(2-((S)- 1-fluoro-2-(methoxy- d3)propan-2-yl)-4- methylpyridin-3-yl)piperidin- 1-yl)pyrimidin-4-yl)-2- methylazetidin-3- yl)piperazine-1-carboxylate | Chiralpak IC, 250 × 21 mm 5 μm, column with a mobile phase of 40% of (3:7) DCM:MeOH using a flow rate of 90 mL/min |
| H-35-1 | 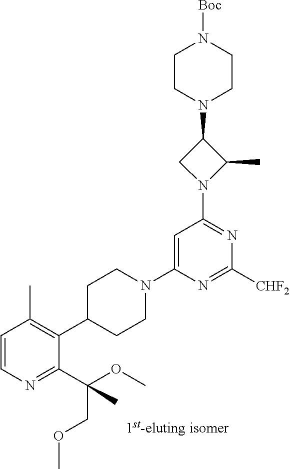 [SMILES: COC[C@@](C)(OC)c1nccc(C)c1C1CCN(c2cc(N3C[C@@H](N4CCN(C(=O)OC(C)(C)C)CC4)[C@H]3C)nc(C(F)F)n2)CC1] | tert-butyl 4-((2R,3R)-1-(2- (difluoromethyl)-6-(4-(2-((S)- 1,2-dimethoxypropan-2-yl)-4- methylpyridin-3-yl)piperidin- 1-yl)pyrimidin-4-yl)-2- methylazetidin-3- yl)piperazine-1-carboxylate | Chiral Pak IC 150 × 50 mm, 5 μ, column with a mobile phase of 30% (1:1) ACN: EtOH using a flow rate of 150 mL/min |
| H-35-2 | 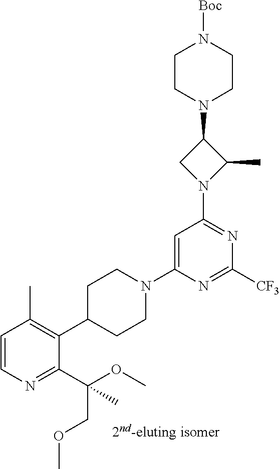 [SMILES: COC[C@](C)(OC)c1nccc(C)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-1-(2- (difluoromethyl)-6-(4-(2-((R)- 1,2-dimethoxypropan-2-yl)-4- methylpyridin-3-yl)piperidin- 1-yl)pyrimidin-4-yl)-2- methylazetidin-3- yl)piperazine-1-carboxylate | Chiral Pack IC 150 × 50 mm, 5 μ, column with a mobile phase of 30% (1:1) ACN: EtOH using a flow rate of 150 mL/min |
| H-36-1 | 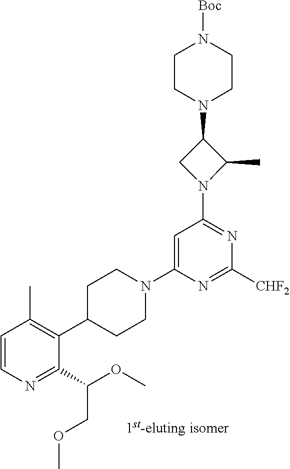 [SMILES: COC[C@@H](OC)c1nccc(C)c1C1CCN(c2cc(N3C[C@@H](N4CCN(C(=O)OC(C)(C)C)CC4)[C@H]3C)nc(C(F)F)n2)CC1] | tert-butyl 4-((2R,3R)-1-(6-(4- (2-((S)-1,2-dimethoxyethyl)- 4-methylpyridin-3- yl)piperidin-1-yl)-2- (trifluoromethyl)pyrimidin-4- yl)-2-methylazetidin-3- yl)piperazine-1-carboxylate | Chiralcel OD-H 250 × 30 mm, 5 μm, column with a mobile phase of 25% IPA using a flow rate of 140 mL/min |
| H-36-2 | 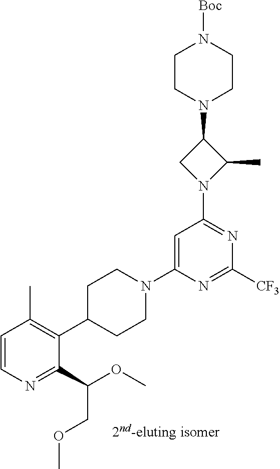 [SMILES: COC[C@H](OC)c1nccc(C)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-1-(6-(4- (2-((R)-1,2-dimethoxyethyl)- 4-methylpyridin-3- yl)piperidin-1-yl)-2- (trifluoromethyl)pyrimidin-4- yl)-2-methylazetidin-3- yl)piperazine-1-carboxylate | Chiralcel OD-H 250 × 30 mm, 5 μm, column with a mobile phase of 25% IPA using a flow rate of 140 mL/min |
| H-37-1 | 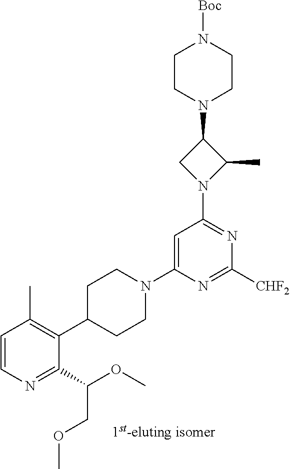 [SMILES: COC[C@@H](OC)c1nccc(C)c1C1CCN(c2cc(N3C[C@@H](N4CCN(C(=O)OC(C)(C)C)CC4)[C@H]3C)nc(C(F)F)n2)CC1] | tert-butyl 4-((2R,3R)-1-(2- (difluoromethyl)-6-(4-(2-((S)- 1,2-dimethoxyethyl)-4- methylpyridin-3-yl)piperidin- 1-yl)pyrimidin-4-yl)-2- methylazetidin-3- yl)piperazine-1-carboxylate | Chiralcel OD-H 250 × 50 mm, 5 μm, column with a mobile phase of 30% (1:1) ACN:IPA using a flow rate of 180 mL/min |
| H-37-2 | 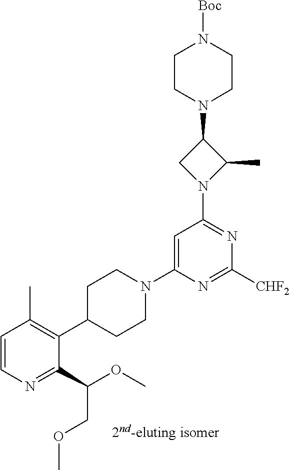 [SMILES: COC[C@H](OC)c1nccc(C)c1C1CCN(c2cc(N3C[C@@H](N4CCN(C(=O)OC(C)(C)C)CC4)[C@H]3C)nc(C(F)F)n2)CC1] | tert-butyl 4-((2R,3R)-1-(2- (difluoromethyl)-6-(4-(2-((R)- 1,2-dimethoxyethyl)-4- methylpyridin-3-yl)piperidin- 1-yl)pyrimidin-4-yl)-2- methylazetidin-3- yl)piperazine-1-carboxylate | Chiralcel OD-H 250 × 50 mm, 5 μm, column with a mobile phase of 30% (1:1) ACN:IPA using a flow rate of 180 mL/min |
| H-38-1 | 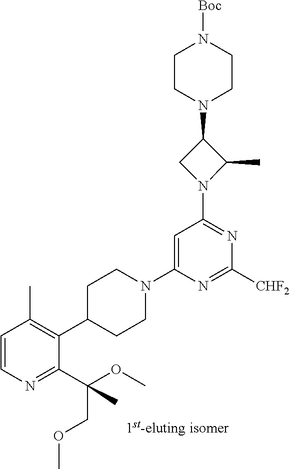 [SMILES: COC[C@@](C)(OC)c1nccc(C)c1C1CCN(c2cc(N3C[C@@H](N4CCN(C(=O)OC(C)(C)C)CC4)[C@H]3C)nc(C(F)F)n2)CC1] | tert-butyl 4-((2R,3R)-1-(6-(4- (2-((S)-1,2-dimethoxypropan- 2-yl)-4-methylpyridin-3- yl)piperidin-1-yl)-2- (trifluoromethyl)pyrimidin-4- yl)-2-methylazetidin-3- yl)piperazine-1-carboxylate | Chiralpak IC, 250 × 50 mm 5 μ, column with a mobile phase of 20% ACN:EtOH (1:1) using a flow rate of 80 mL/min |
| H-38-2 | 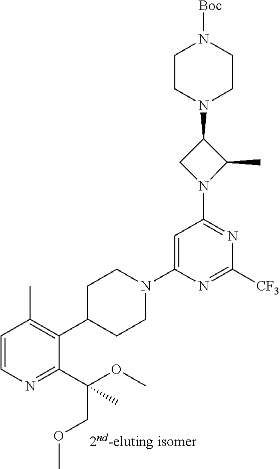 [SMILES: COC[C@](C)(OC)c1nccc(C)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-1-(6-(4- (2-((R)-1,2-dimethoxypropan- 2-yl)-4-methylpyridin-3- yl)piperidin-1-yl)-2- (trifluoromethyl)pyrimidin-4- yl)-2-methylazetidin-3- yl)piperazine-1-carboxylate | Chiralpak IC, 250 × 50 mm 5 μ, column with a mobile phase of 20% ACN:EtOH (1:1) using a flow rate of 80 mL/min |
| H-39-1 | 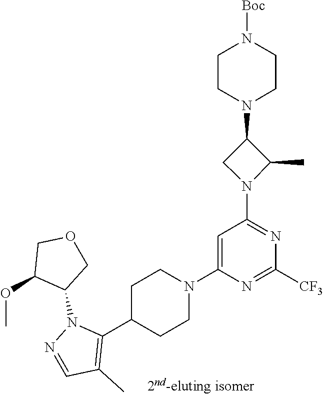 [SMILES: CO[C@H]1COC[C@@H]1n1ncc(C)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-1-(6-(4- (1-((3S,4R)-4- methoxytetrahydrofuran-3- yl)-4-methyl-1H-pyrazol-5- yl)piperidin-1-yl)-2- (trifluoromethyl)pyrimidin-4- yl)-2-methylazetidin-3- yl)piperazine-1-carboxylate | LUX-C4, 250 × 50 mm, 5 μm, column with a mobile phase of 40% (8:2) ACN:IPA using a flow rate of 180 mL/min |
| H-39-2 | 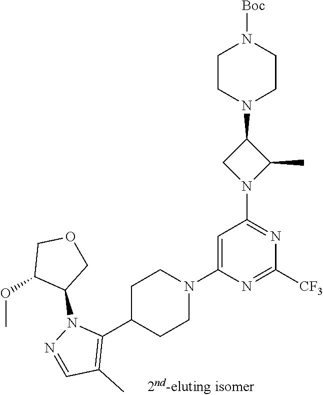 [SMILES: CO[C@@H]1COC[C@H]1n1ncc(C)c1C1CCN(c2cc(N3C[C@@H](N4CCN(C(=O)OC(C)(C)C)CC4)[C@H]3C)nc(C(F)(F)F)n2)CC1] | tert-butyl 4-((2R,3R)-1-(6-(4- (1-((3R,4S)-4- methoxytetrahydrofuran-3- yl)-4-methyl-1H-pyrazol-5- yl)piperidin-1-yl)-2- (trifluoromethyl)pyrimidin-4- yl)-2-methylazetidin-3- yl)piperazine-1-carboxylate | LUX-C4, 250 × 50 mm, 5 μm, column with a mobile phase of 40% (8:2) ACN:IPA using a flow rate of 180 mL/min |
| H-40-1 | 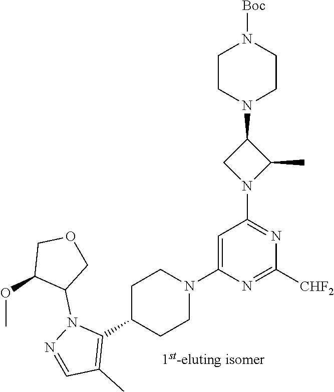 | tert-butyl 4-((2R,3R)-1-(2- (difluoromethyl)-6-(4-(1- ((3S,4R)-4- methoxytetrahydrofuran-3- yl)-4-methyl-1H-pyrazol-5- yl)piperidin-1-yl)pyrimidin-4- yl)-2-methylazetidin-3- yl)piperazine-1-carboxylate | LUX-C4, 250 × 50 mm, 5 μm, column with a mobile phase of 50% (8:2) ACN:IPA using a flow rate of 180 mL/min |
| H-40-2 | 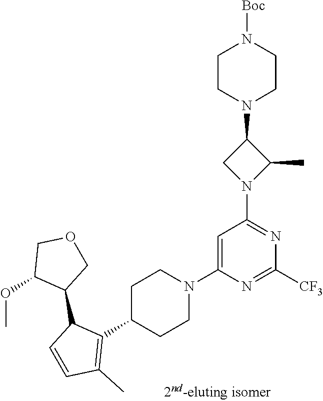 | tert-butyl 4-((2R,3R)-1-(2- (difluoromethyl)-6-(4-(1- ((3R,4S)-4- methoxytetrahydrofuran-3- yl)-4-methyl-1H-pyrazol-5- yl)piperidin-1-yl)pyrimidin-4- yl)-2-methylazetidin-3- yl)piperazine-1-carboxylate | LUX-C4, 250 × 50 mm, 5 μm, column with a mobile phase of 50% (8:2) ACN:IPA using a flow rate of 180 mL/min |
| H-41-1 | 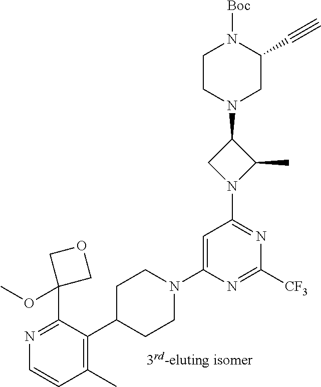 [SMILES: C#C[C@@H]1CN([C@@H]2CN(c3cc(N4CCC(c5c(C)ccnc5C5(OC)COC5)CC4)nc(C(F)(F)F)n3)[C@@H]2C)CCN1C(=O)OC(C)(C)C] | tert-butyl (R)-2-ethynyl-4- ((2R,3R)-1-(6-(4-(2-(3- methoxyoxetan-3-yl)-4- methylpyridin-3-yl)piperidin- 1-yl)-2- (trifluoromethyl)pyrimidin-4- yl)-2-methylazetidin-3- yl)piperazine-1-carboxylate | ChiralPak IC, 2 × 25 cm 5 μm column with a mobile phase of 40% iPrOH using a flow rate of 80 mL/min |
| H-42-1 | 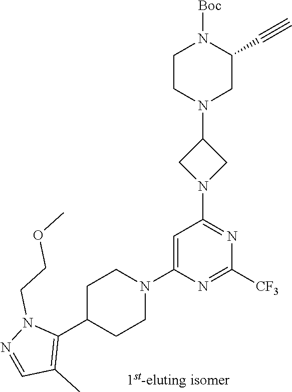 [SMILES: C#C[C@@H]1CN(C2CN(c3cc(N4CCC(c5c(C)cnn5CCOC)CC4)nc(C(F)(F)F)n3)C2)CCN1C(=O)OC(C)(C)C] | tert-butyl (R)-2-ethynyl-4-(1- (6-(4-(1-(2-methoxyethyl)-4- methyl-1H-pyrazol-5- yl)piperidin-1-yl)-2- (trifluoromethyl)pyrimidin-4- yl)azetidin-3- yl)piperazine-1-carboxylate | ChiralPak IG, 2 × 25 cm 5 μm column with a mobile phase of 35% iPrOH using a flow rate of 80 mL/min |
| H-43-1 | 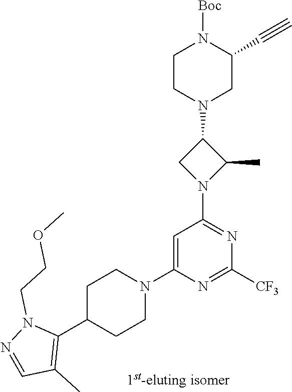 [SMILES: C#C[C@@H]1CN([C@H]2CN(c3cc(N4CCC(c5c(C)cnn5CCOC)CC4)nc(C(F)(F)F)n3)[C@@H]2C)CCN1C(=O)OC(C)(C)C] | tert-butyl 2-ethynyl-4- ((2R,3S)-1-(6-(4-(1-(2- methoxyethyl)-4-methyl-1H- pyrazol-5-yl)piperidin-1-yl)- 2-(trifluoromethyl)pyrimidin- 4-yl)-2-methylazetidin-3- yl)piperazine-1-carboxylate | ChiralPak IC, 2 × 25 cm 5 μm column with a mobile phase of 25% MeOH using a flow rate of 80 mL/min |
| H-43-2 | 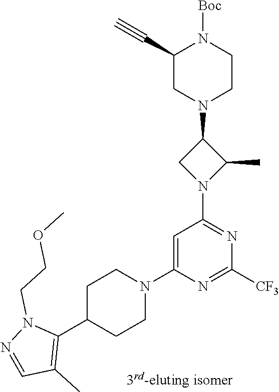 [SMILES: C#C[C@@H]1CN([C@@H]2CN(c3cc(N4CCC(c5c(C)cnn5CCOC)CC4)nc(C(F)(F)F)n3)[C@@H]2C)CCN1C(=O)OC(C)(C)C] | tert-butyl (R)-2-ethynyl-4- ((2R,3R)-1-(6-(4-(1-(2- methoxyethyl)-4-methyl-1H- pyrazol-5-yl)piperidin-1-yl)- 2-(trifluoromethyl)pyrimidin- 4-yl)-2-methylazetidin-3- yl)piperazine-1-carboxylate | Chiralpak IC, 21 × 150 mm 5 μm, column with a mobile phasse of 40% 2-propanol using a flow rate of 140 mL/min |
| H-44-1 | 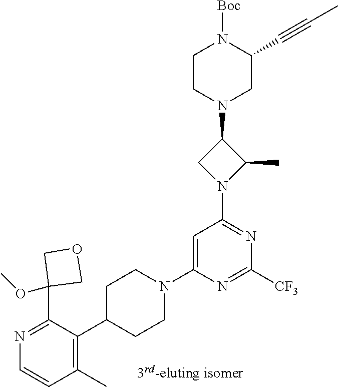 [SMILES: CC#C[C@@H]1CN([C@@H]2CN(c3cc(N4CCC(c5c(C)ccnc5C5(OC)COC5)CC4)nc(C(F)(F)F)n3)[C@@H]2C)CCN1C(=O)OC(C)(C)C] | tert-butyl (R)-4-((2R,3R)-1- (6-(4-(2-(3-methoxyoxetan-3- yl)-4-methylpyridin-3- yl)piperidin-1-yl)-2- (trifluoromethyl)pyrimidin-4- yl)-2-methylazetidin-3-yl)-2- (prop-1-yn-1- yl)piperazine-1-carboxylate | (S,S) Whelk-0, 2 × 25 cm 5 μm column with a mobile phase of 30% iPrOH using a flow rate of 100 mL/min |
| H-45-1 | 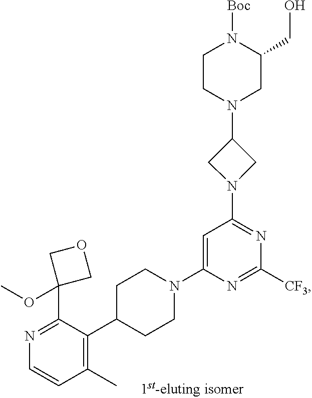 [SMILES: COC1(c2nccc(C)c2C2CCN(c3cc(N4CC(N5CCN(C(=O)OC(C)(C)C)[C@H](CO)C5)C4)nc(C(F)(F)F)n3)CC2)COC1] | tert-butyl (S)-2- (hydroxymethyl)-4-(1-(6-(4- (2-(3-methoxyoxetan-3-yl)-4- methylpyridin-3-yl)piperidin- 1-yl)-2- (trifluoromethyl)pyrimidin-4- yl)azetidin-3- yl)piperazine-1-carboxylate | Chiralpak IC, 150 × 50 mm 5 μm, column with a mobile phase of 50% (1:1) ACN:MeOH using a flow rate of 190 mL/min |
| H-46-1 | 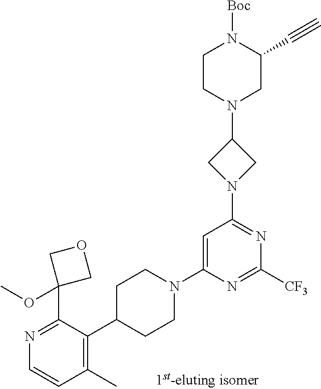 [SMILES: C#C[C@@H]1CN(C2CN(c3cc(N4CCC(c5c(C)ccnc5C5(OC)COC5)CC4)nc(C(F)(F)F)n3)C2)CCN1C(=O)OC(C)(C)C] | tert-butyl (R)-2-ethynyl-4-(1- (6-(4-(2-(3-methoxyoxetan-3- yl)-4-methylpyridin-3- yl)piperidin-1-yl)-2- (trifluoromethyl)pyrimidin-4- yl)azetidin-3- yl)piperazine-1-carboxylate | Chiralpak AD-H 150 × 50 mm, 5 μm, column with a mobile phase of 35% IPA using a flow rate of 180 mL/min |
| H-46-2 | 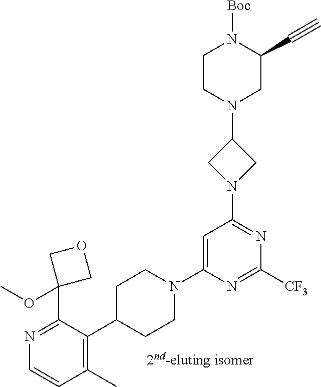 [SMILES: C#C[C@H]1CN(C2CN(c3cc(N4CCC(c5c(C)ccnc5C5(OC)COC5)CC4)nc(C(F)(F)F)n3)C2)CCN1C(=O)OC(C)(C)C] | tert-butyl (S)-2-ethynyl-4-(1- (6-(4-(2-(3-methoxyoxetan-3- yl)-4-methylpyridin-3- yl)piperidin-1-yl)-2- (trifluoromethyl)pyrimidin-4- yl)azetidin-3- yl)piperazine-1-carboxylate | Chiralpak AD-H 150 × 50 mm, 5 μm, column with a mobile phase of 35% IPA using a flow rate of 180 mL/min |
| H-47-1 | 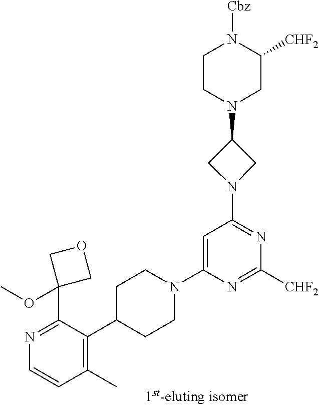 | benzyl (S)-2- (difluoromethyl)-4-(1-(6-(4- (2-(3-methoxyoxetan-3-yl)-4- methylpyridin-3-yl)piperidin- 1-yl)-2- (trifluoromethyl)pyrimidin-4- yl)azetidin-3- yl)piperazine-1-carboxylate | Chiralpak OD-H 250 × 50 mm, 5 μm, column with a mobile phase of 20% MeOH using a flow rate of 180 mL/min |

### Example 2-130:1-((S)-4-((2R,3R)-1-(6-(4-(1,4-dimethyl-1H-pyrazol-5-yl)piperidin-1-yl)-2-(trifluoromethyl)pyrimidin-4-yl)-2-methyl)azetidin-3-yl)-2-(fluoromethyl)piperazin-1-yl)prop-2-en-1-one

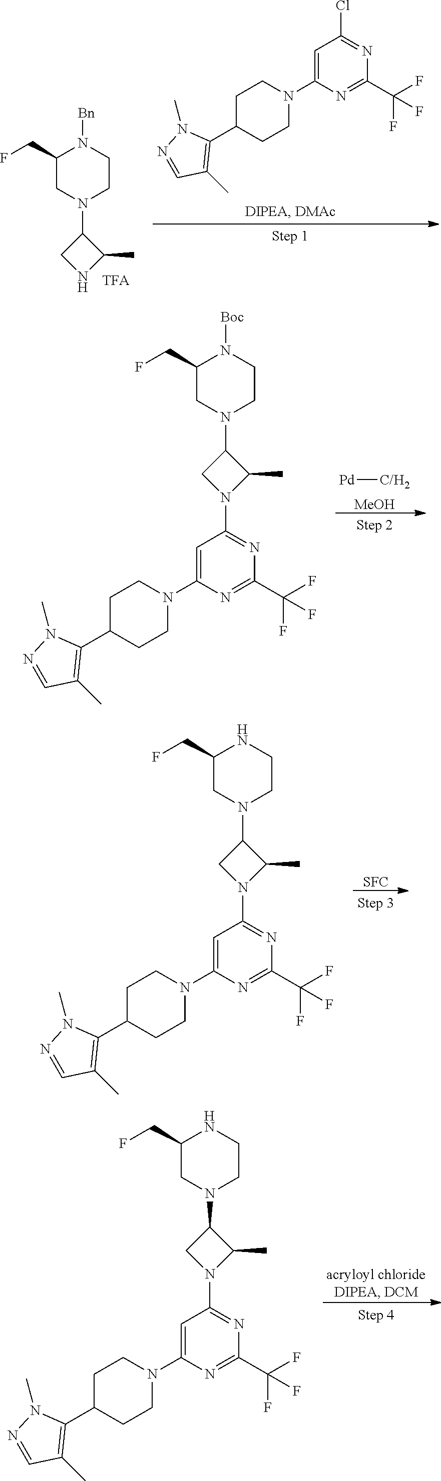

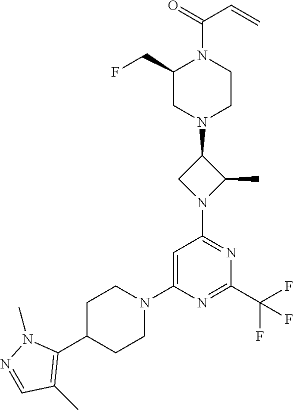 [SMILES: C=CC(=O)N1CCN([C@@H]2CN(c3cc(N4CCC(c5c(C)cnn5C)CC4)nc(C(F)(F)F)n3)[C@@H]2C)C[C@H]1CF]

Step 1. Toa solution of (2S)-1-benzyl-2-(fluoromethyl)-((2R)-2-methyl)azetidin-3-yl)piperazine trifluoroacetate salt ((0.312 g, 0.834 mmol) in DMA (4 mL) were added DIPEA (0.971 mL, 5.56 mmol, Chempure) and 4-chloro-6-(4-(1,4-dimethyl-1H-pyrazol-5-yl)piperidin-1-yl)-2-(trifluoromethyl)pyrimidine (0.2 g, 0.556 mmol) at rt. The resulting mixture was stirred for 16 h at 95° C. The reaction mixture was quenched with H₂O (20 mL) and extracted with EtOAc (3×20) mL). Combined organic extracts were washed with brine, dried over anhydrous Na₂SO₄, filtered, and concentrated under reduced pressure. The crude residue was purified by chromatography (silica, 40 to 60% EtOAc:hexanes) to obtain 4-((2R,3R)-3-((S)-4-benzyl-3-(fluoromethyl)piperazin-1-yl)-2-methyl)azetidin-1-yl)-6-(4-(1,4-dimethyl-1H-pyrazol-5-yl)piperidin-1-yl)-2-(trifluoromethyl)pyrimidine. m/z (ESI): 601.2 (M+H)⁺.

Step 2. To a stirred solution of 4-((2R,3R)-3-((S)-4-benzyl-3-(fluoromethyl)piperazin-1-yl)-2-methyl)azetidin-1-yl)-6-(4-(1,4-dimethyl-1H-pyrazol-5-yl)piperidin-1-yl)-2-(trifluoromethyl)pyrimidine (0.3 g, 0.499 mmol) in MeOH (10 mL) was added 10% Pd—C (0.027 g, 0.025 mmol, Sigma-Aldrich, Inc.) under N₂ atmosphere. The reaction mixture was degassed and stirred under 1 atm H₂ atmosphere for 16 h at 25° C. The reaction mixture was filtered through a celite pad and washed with MeOH (20 mL). The filtrate was concentrated under reduced pressure and purified by chromatography (silica, 3 to 5% MeOH:DCM) to obtain 4-(4-(1,4-dimethyl-1H-pyrazol-5-yl)piperidin-1-yl)-6-((2R,3R)-3-((S)-3-(fluoromethyl)piperazin-1-yl)-2-methyl)azetidin-1-yl)-2-(trifluoromethyl)pyrimidine. m/z (ESI): 511.4 (M+H)⁺

Step 3. The mixture of isomers, 180 mg was dissolved in 20 mL of MeCN/MeOH (1:1) added few drops of DEA. Separation was performed in an SFC-BT-200 instrument on a Chiralpak AS-H (250×30) mm, 5 μm column with CO₂:(0.2% DEA in MeOH) (85.15) at a flow rate of 150 mL/min. The configuration was assigned arbitrarily. Peak 1 trace amount. m/z (ESI): 511.4 (M+H)⁺. Peak 2.30 mg. m/z (ESI): 511.4 (M+H)⁺

Step 4. To a stirred solution of 4-(4-(1,4-dimethyl-1H-pyrazol-5-yl)piperidin-1-yl)-6-((2R,3R)-3-((S)-3-(fluoromethyl)piperazin-1-yl)-2-methyl)azetidin-1-yl)-2-(trifluoromethyl)pyrimidine (0.03 g, 0.059 mmol) in DCM (1.0 mL) at −78° C. was added DIPEA (0.051 mL, 0.294 mmol) and stirred for 5 min. Then, acryloyl chloride (4.77 μL, 0.059 mmol) in DCM (0.5 mL) was added dropwise at −78° C. and stirred for 10 min. The reaction mixture was quenched with H₂O (10 mL) and allowed to warm to rt. The organic phase was extracted with DCM (10 mL×3). combined, dried over Na₂SO₄, and concentrated under reduced pressure. The crude material was purified by reverse phase preparative HPLC (YMC C18 (250.20 mm) 5.0 μm column with a mobile phase of 0.1% NH₃ in H₂O and ACN using a flow rate of 15 mL/min) followed by lyophilization to give 1-((S)-4-((2R,3R)-1-(6-(4-(1,4-dimethyl-1H-pyrazol-5-yl)piperidin-1-yl)-2-(trifluoromethyl)pyrimidin-4-yl)-2-methyl)azetidin-3-yl)-2-(fluoromethyl)piperazin-1-yl)prop-2-en-1-one. m/z (ESI): 565.3 (M+H)⁺. ¹H NMR (400 MHz, DMSO-d₆) δ ppm 7.07 (s, 1H), 6.79 (dd, 1H, J=16.7, 10.5 Hz), 6.13 (dd, 1H, J=16.6, 2.4 Hz), 5.75-5.8 (m, 2H), 4.4-4.9 (m, 6H), 4.28 (d, 1H, J=13.8 Hz), 3.94 (t, 1H, J=8.3 Hz), 3.81 (t, 1H, J=7.8 Hz), 3.77 (s, 3H), 3.2-3.3 (m, 1H), 3.0-3.2 (m, 1H), 2.9-3.0 (m, 2H), 2.7-2.9 (m, 2H), 2.53 (s, 1H), 1.7-2.1 (m, 9H), 1.39 (d, 3H, J=6.3 Hz).

The examples in the table below were prepared in a manner similar to that described above.

| | | | m/z (ESD): |
|---|---|---|---|
| Ex. | Chemical Structure | Name | (M + H)⁺ |
| 2-131 | 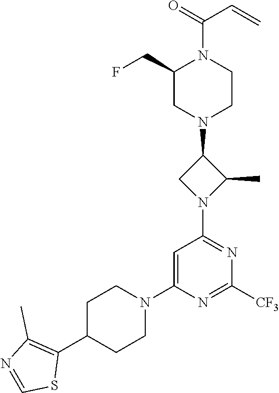 [SMILES: C=CC(=O)N1CCN([C@@H]2CN(c3cc(N4CCC(c5scnc5C)CC4)nc(C(F)(F)F)n3)[C@@H]2C)C[C@H]1CF] | 1-((S)-2-(fluoromethyl)-4- ((2R,3S)-2-methyl-1-(6-(4-(4- methylthiazol-5-yl)piperidin-1- yl)-2- (trifluoromethyl)pyrimidin-4- yl)azetidin-3-yl)piperazin-1- yl)prop-2-en-1-one | 568.2 |
| 2-132 | 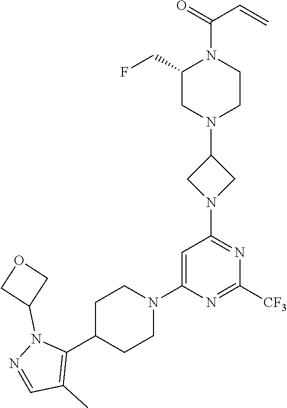 [SMILES: C=CC(=O)N1CCN(C2CN(c3cc(N4CCC(c5c(C)cnn5C5COC5)CC4)nc(C(F)(F)F)n3)C2)C[C@@H]1CF] | (R)-1-(2-(fluoromethyl)-4-(1- (6-(4-(4-methyl-1-(oxetan-3- yl)-1H-pyrazol-5-yl)piperidin- 1-yl)-2- (trifluoromethyl)pyrimidin-4- yl)azetidin-3-yl)piperazin-1- yl)prop-2-en-1-one | 593.2 |
| 2-133 | 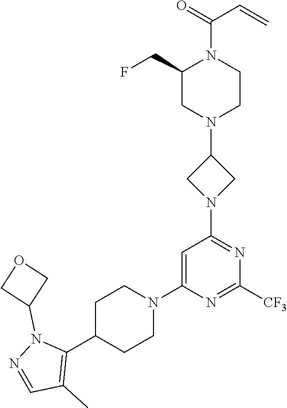 [SMILES: C=CC(=O)N1CCN(C2CN(c3cc(N4CCC(c5c(C)cnn5C5COC5)CC4)nc(C(F)(F)F)n3)C2)C[C@H]1CF] | (S)-1-(2-(fluoromethyl)-4-(1- (6-(4-(4-methyl-1-(oxetan-3- yl)-1H-pyrazol-5-yl)piperidin- 1-yl)-2- (trifluoromethyl)pyrimidin-4- yl)azetidin-3-yl)piperazin-1- yl)prop-2-en-1-one | 593.2 |
| 2-134 | 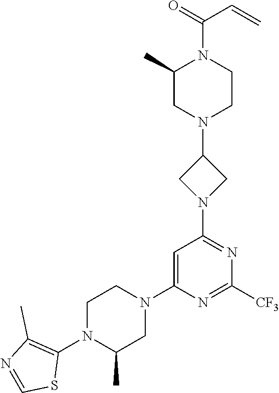 [SMILES: C=CC(=O)N1CCN(C2CN(c3cc(N4CCN(c5scnc5C)[C@H](C)C4)nc(C(F)(F)F)n3)C2)C[C@H]1C] | 1-((R)-2-methyl-4-(1-(6-((R)- 3-methyl-4-(4-methylthiazol- 5-yl)piperazin-1-yl)-2- (trifluoromethyl)pyrimidin-4- yl)azetidin-3-yl)piperazin-1- yl)prop-2-en-1-one | 551.3 |
| 2-425 | 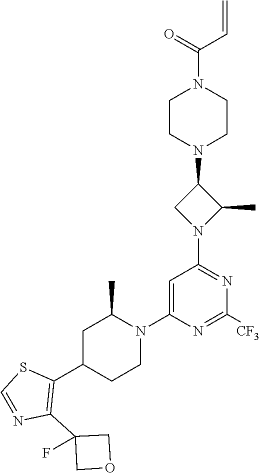 [SMILES: C=CC(=O)N1CCN([C@@H]2CN(c3cc(N4CCC(c5scnc5C5(F)COC5)C[C@H]4C)nc(C(F)(F)F)n3)[C@@H]2C)CC1] | 1-(4-((2R,3R)-1-(2-((difluoro- l3-methyl)-l2-fluoraneyl)-6- ((2R)-4-(4-(3-fluorooxetan-3- yl)thiazol-5-yl)-2- methylpiperidin-1- yl)pyrimidin-4-yl)-2- methylazetidin-3-yl)piperazin- 1-yl)prop-2-en-1-one | 610.5 |
| 2-642 | 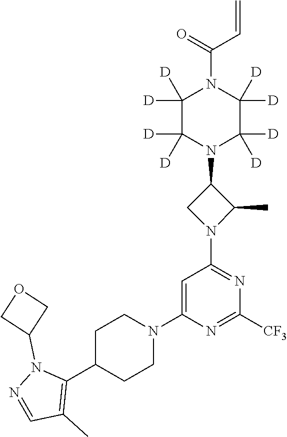 [SMILES: [2H]C1([2H])N(C(=O)C=C)C([2H])([2H])C([2H])([2H])N([C@@H]2CN(c3cc(N4CCC(c5c(C)cnn5C5COC5)CC4)nc(C(F)(F)F)n3)[C@@H]2C)C1([2H])[2H]] | 1-(4-((2R,3R)-2-methyl-1-(6- (4-(4-methyl-1-(oxetan-3-yl)- 1H-pyrazol-5-yl)piperidin-1- yl)-2- (trifluoromethyl)pyrimidin-4- yl)azetidin-3-yl)piperazin-1-yl- 2,2,3,3,5,5,6,6-d8)prop-2-en-1- one | 583.2 |

### SFC Conditions for Chiral Separation

| | | | Separation |
|---|---|---|---|
| Ex. | Chemical Structure | Name | condition |
| 2-425-1 | 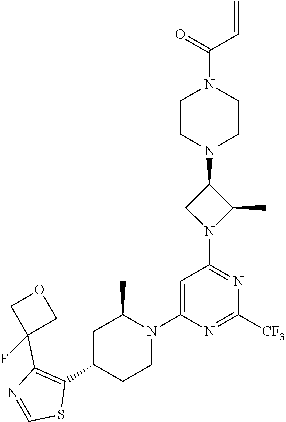 [SMILES: C=CC(=O)N1CCN([C@@H]2CN(c3cc(N4CC[C@H](c5scnc5C5(F)COC5)C[C@H]4C)nc(C(F)(F)F)n3)[C@@H]2C)CC1] | 1-(4-((2R,3R)-1-(6- ((2R,4SR)-4-(4-(3-fluoro- 3-oxetanyl)-1,3-thiazol-5- yl)-2-methyl-1- piperidinyl)-2- (trifluoromethyl)-4- pyrimidinyl)-2-methyl-3- azetidinyl)-1-piperazinyl)- 2-propen-1-one | 2-425 \| Chiralpak IG 250 × 30 mm, 10 μm column with a mobile phase of 50 % EtOH (0.1% NH₃) in liquid CO₂ with a flow rate of 70 mL/min |
| | 2^{nd}-eluting isomer | | |

### Example 2-135: 1-(4-((2S,3R)-2-methyl-1-(6-(4-(4-methyl-1-(oxetan-3-yl)-1H-pyrazol-5-yl)piperidin-1-yl)-2-(trifluoromethyl)pyrimidin-4-yl)azetidin-3-yl)piperazin-1-yl)prop-2-en-1-one

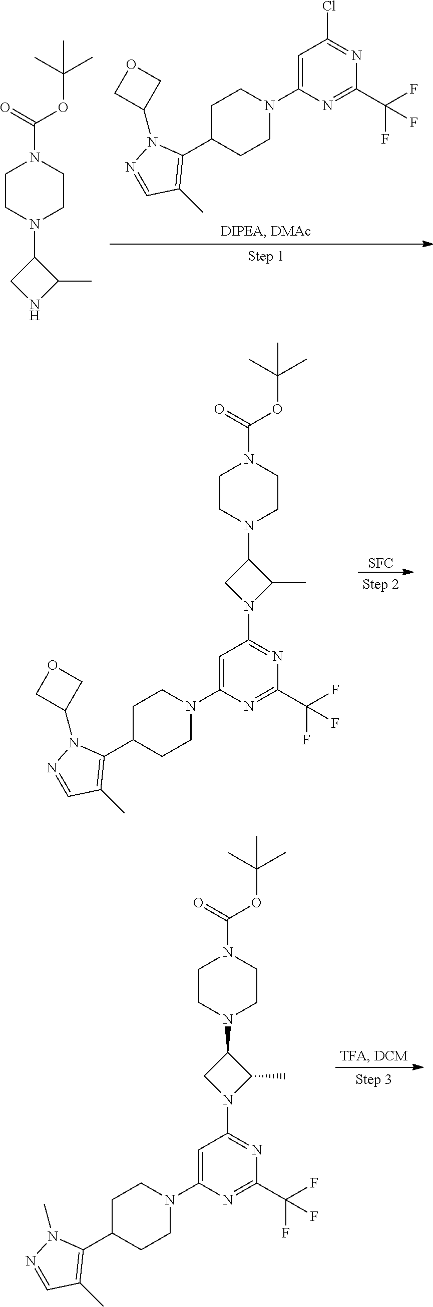

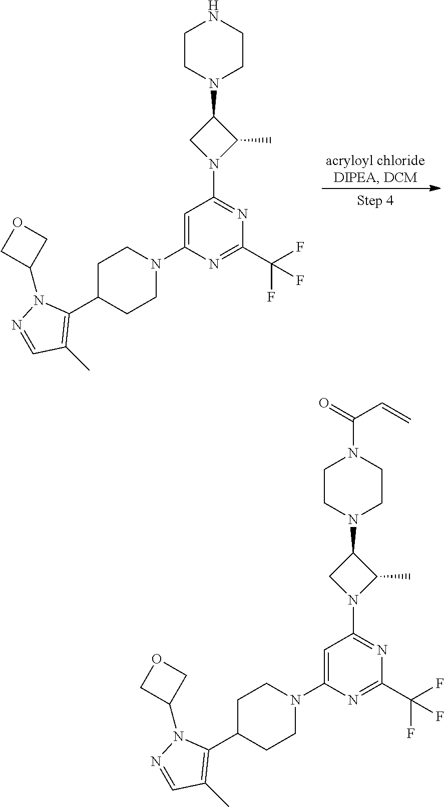 [SMILES: Cc1cnn(C2COC2)c1C1CCN(c2cc(N3C[C@@H](N4CCNCC4)[C@@H]3C)nc(C(F)(F)F)n2)CC1 ; "acryloyl chlorideDIPEA, DCM" ; C=CC(=O)N1CCN([C@@H]2CN(c3cc(N4CCC(c5c(C)cnn5C5COC5)CC4)nc(C(F)(F)F)n3)[C@H]2C)CC1]

Step 1. To a stirred solution of tert-butyl 4-(2-methyl)azetidin-3-yl)piperazine-1-carboxylate (0.114 g, 0.448 mmol) in DMA (1 mL) was added 4-chloro-6-(4-(4-methyl-1-(oxetan-3-yl)-1H-pyrazol-5-yl)piperidin-1-yl)-2-(trifluoromethyl)pyrimidine (0.18 g, 0.448 mmol) and DIPEA (0.391 mL, 2.240 mmol). The reaction mixture was stirred at 100° C. for 16 h. After cooling to rt, the reaction mixture was poured into with ice cold water and extracted with EtOAc (2×50 mL). The combined organic extracts were washed with brine (50 mL) and dried over Na₂SO₄. The solution was filtered and concentrated under reduced pressure. The crude residue was purified on silica gel eluting with 80% to 90% EtOAc in pet ether to give tert-butyl 4-(2-methyl-1-(6-(4-(4-methyl-1-(oxetan-3-yl)-1H-pyrazol-5-yl)piperidin-1-yl)-2-(trifluoromethyl)pyrimidin-4-yl)azetidin-3-yl piperazine-1-carboxylate.

Step 2. The mixture of isomers, 160 mg was dissolved in 4 mL of MeCN/MeOH (1:1). Separation was performed in an SFC-BT-200 instrument on a Chiralpak IC (250×21) mm, 5 μm column liquid C02: [MeCN:IPA (1:1)] (75:25) at a flow rate of 120 mL/min. The configuration was assigned arbitrarily. Peak 1: m/z (ESI): 620.7 (M+H)⁺. Peak 2: m/z (ESI): 621.6 (M+H)⁺

Step 3. To a solution of ten-butyl 4-((2S,3R)-2-methyl-1-(6-(4-(4-methyl-1-(oxetan-3-yl)-1H-pyrazol-5-yl)piperidin-1-yl)-2-(trifluoromethyl)pyrimidin-4-yl)azetidin-3-yl)piperazine-1-carboxylate (30 mg, 0.048 mmol) (peak 1) in DCM (3 mL) at 0° C. was added TFA (37.2 μL, 0.483 mmol) dropwise and the reaction mixture was stirred at rt for 2 h. The solvent was removed under vacuum and the crude material was carried to next step.

Step 4. To a solution of 4-(4-(4-methyl-1-(oxetan-3-yl)-1H-pyrazol-5-yl)piperidin-1-yl)-6-((2S,3R)-2-methyl-3-(piperazin-1-yl)azetidin-1-yl)-2-(trifluoromethyl)pyrimidine (25 mg, 0.048 mmol) in DCM (5 mL) at 0° C. was added TEA (26.8 μL, 0.192 mmol) followed by acryloyl chloride (5.22 mg, 0.058 mmol). The reaction mixture was stirred at 0° C. for 5 mm. The reaction mixture is diluted with H₂O and extracted with DCM (3×10 mL). The combined organic extracts were dried over Na₂SO₄ and concentrated under vacuum. The crude residue was purified by prep HPLC to give 1-(4-((2S,3R)-2-methyl-1-(6-(4-(4-methyl-1-(oxetan-3-yl)-1H-pyrazol-5-yl)piperidin-1-yl)-2-(trifluoromethyl)pyrimidin-4-yl)azetidin-3-yl)piperazin-1-yl)prop-2-en-1-one. m/z (ESI): 574.7 (M+H)⁺. ¹H NMR (DMSO-d₆, 401 MHz): δ (ppm) 7.30 (s, 1H), 6.80 (dd, J=16.7, 10.5 Hz, 1H), 6.11 (dd, J=16.7, 2.4 Hz, 1H), 5.75-5.63 (m, 3H), 4.96-4.83 (m, 4H), 4.47 (d, J=13.0 Hz, 2H), 4.23-4.15 (m, 1H), 4.05 (dd, J=8.8, 7.0 Hz, 1H), 3.64 (dd, J=9.0, 5.3 Hz, 1H), 3.55 (d, J=13.8 Hz, 4H), 3.03 (dt, J=10.9, 5.5 Hz, 1H), 293 (t, J=11.6 Hz, 2H), 2.85-2.77 (m, 1H), 2.32 (s, 5H), 1.99 (s, 3H), 1.74-1.67 (m, 4H), 1.44 (d, J=6.3 Hz, 3H).

### Example 2-136: 1-(4-((2R,3R)-2-Methyl-1-(6-(4-(4-methyl-1-(1-methylpyrrolidin-3-yl)-1H-pyrazol-5-yl)piperidin-1-yl)-2-(trifluoromethyl)pyrimidin-4-yl)azetidin-3-yl)piperazin-1-yl)prop-2-en-1-one

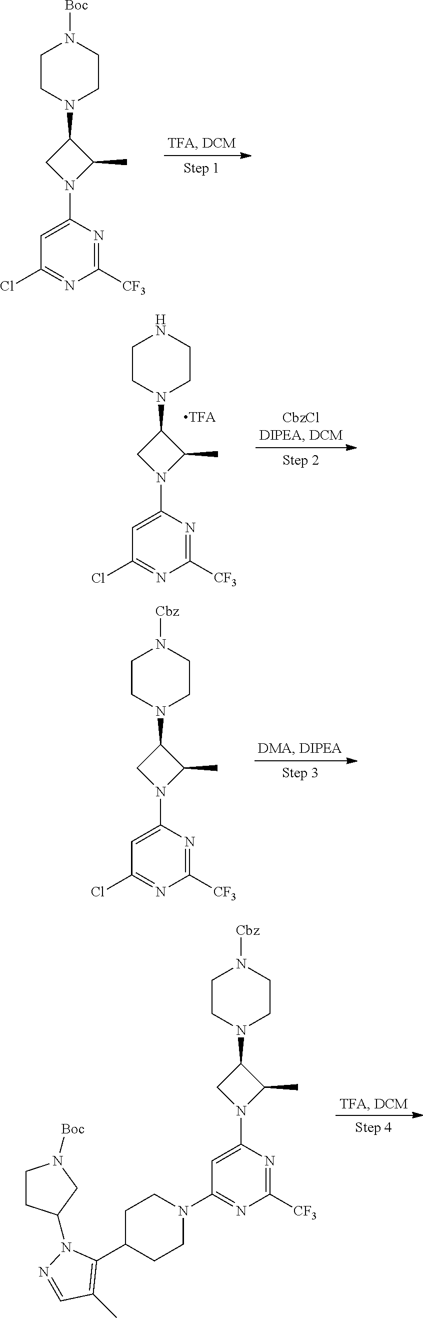

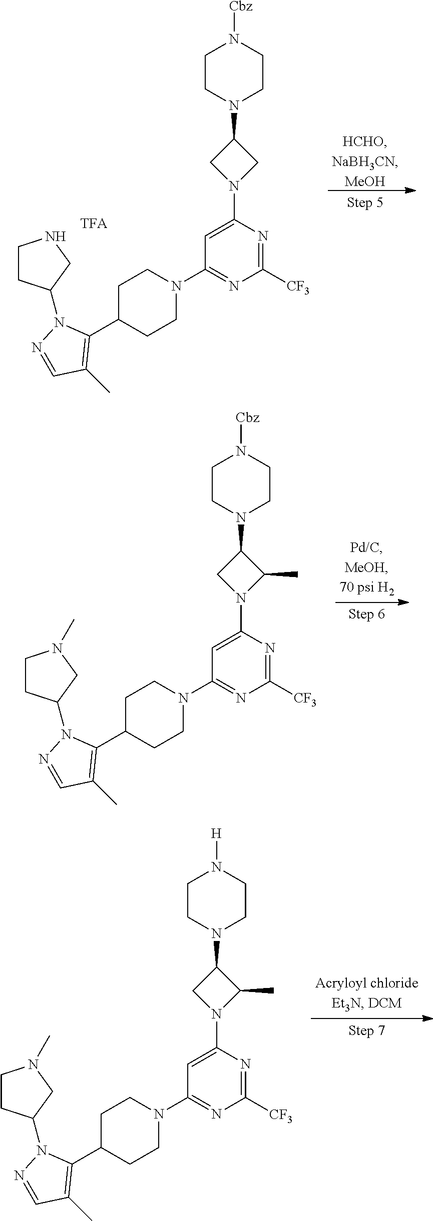

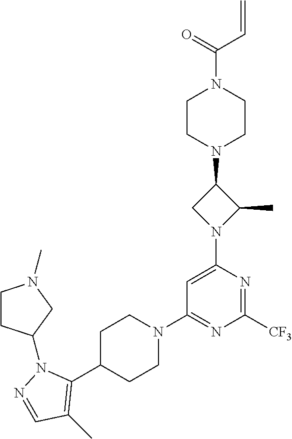 [SMILES: C=CC(=O)N1CCN([C@@H]2CN(c3cc(N4CCC(c5c(C)cnn5C5CCN(C)C5)CC4)nc(C(F)(F)F)n3)[C@@H]2C)CC1]

Step 1. Toa solution of tert-butyl 2R,3R)-1-(6-chloro-2-(trifluoromethyl)pyrimidin-4-yl)-2-methyl)azetidin-3-yl)piperazine-1-carboxylate (0.5 g, 1.147 mmol) in DCM (5 mL) was added TFA (0.088 mL, 1.147 mmol) at 0° C. The reaction mixture was stirred at rt for 3 ht. concentrated, coevaporated with toluene (25 mL), and triturated with diethyl ether to give 4-chloro-6-((2R,3R)-2-methyl-3-(piperazin-1-yl)azetidin-1-yl)-2-(trifluoromethyl)pyrimidine trifluoroacetate salt. The crude residue was taken to the next step without further purification.

Step 2. To a solution of 4-chloro-6-((2R,3R)-2-methyl-3-(piperazin-1-yl)azetidin-1-yl)-2-(trifluoromethylpyrimidine trifluoroacetate salt (0.385 g, 1.47 mmol) in DCM (10 mL at e TF were added DIPEA (0.601 mL, 3.44 mmol, Spectrochem) and CbzCl (0.196 mL, 1.376 mmol) and stirred at rt for 16 h. The reaction mixture was quenched with ice water (15 mL) and extracted with EtOAc (3×30 mL). The combined organic extracts were washed with ice water (20 mL) followed by brine (20 mL). dried over Na₂SO₄, and concentrated to give crude benzyl 4-((2R,3R)-1-(6-chloro-2-(trifluoromethyl)pyrimidin-4-yl)-2-methyl)azetidin-3-yl)piperazine-1-carboxylate. m/z (ESI): 470.1 (M+H)⁺.

Step 3. To a solution of benzyl 4-((2R,3R)-1-(6-chloro-2-(trifluoromethyl)pyrimidin-4-yl)-2-methyl)azetidin-3-yl)piperazine-1-carboxylate (0.45 g, 0.958 mmol) in DMA (5 mL) at 0° C. were added DIPEA (0.836 mL, 4.79 mmol, Spectrochem) and tert-butyl 3-(4-methyl-5-(piperidin-4-yl)-1H-pyrazol-1-yl)pyrrolidine-1-carboxylate (0.320 g, 0.958 mmol) and stirred at 100° C. for 16 h. The reaction mixture was quenched with ice water (15 mL) and extracted with EtOAc (3.30 mL). The combined organic extracts were washed with ice water (20 mL) followed by brine (20 mL), dried over Na₂SO₄, and concentrated. The crude residue was purified by column chromatography (silica, 25-50% EtOAc in pet ether) to give benzyl 4-((2R)-1-(6-(4-(1-(1-(tert-butoxycarbonyl)pyrrolidin-3-yl)-4-methyl-1H-pyrazol-5-yl)piperidin-1-yl)-2-(trifluoromethyl)pyrimidin-4-yl)-2-methyl)azetidin-3-yl)piperazine-1-carboxylate. m/z (ESI): 768.3 (M+H)⁺.

Step 4. To a solution of benzyl 4-((2R)-1-(6-(4-(1-(1-(tert-butoxycarbonyl)pyrrolidin-3-yl)-4-methyl-1H-pyrazol-5-yl)piperidin-1-yl)-2-(trifluoromethyl)pyrimidin-4-yl)-2-methyl)azetidin-3-yl)piperazine-1-carboxylate (0.261 g, 0.340 mmol) in DCM (5 mL) was added TFA (0.223 g, 1.953 mmol) at 0° C. The reaction mixture was stirred at rt for 3 h. The reaction mixture was concentrated, co-evaporated with toluene (25 mL), and triturated with diethyl ether to give the crude benzyl 4-((2R)-2-methyl-1-(6-(4-(4-methyl-1-(pyrrolidin-3-yl)-1H-pyrazol-5-yl)piperidin-1-yl)-2-(trifluoromethyl)pyrimidin-4-yl)azetidin-3-yl)piperazine-1-carboxylate trifluoroacetate salt. The crude residue was taken to the next step without further purification. m/z (ESI): 668.3 (M+H)⁺.

Step 5. To a solution of benzyl 4-((2R)-2-methyl-1-(6-(4-(4-methyl-1-(pyrrolidin-3-yl)-1H-pyrazol-5-yl)piperidin-1-yl)-2-(trifluoromethyl)pyrimidin-4-yl)azetidin-3-yl)piperazine-1-carboxylate trifluoroacetate salt (0.261 g, 0.391 mmol) in MeOH (10 mL) was added formaldehyde (0.196 g, 1.954 mmol) and the reaction mixture was stirred at rt for 16 h, then sodium cyanoborohydride (0.123 g, 1.954 mmol) was added and allowed to stir until completion. The reaction mixture was quenched with ice water (15 mL) and extracted with EtOAc (3×30 mL). The combined organic extracts were washed with ice water (20 mL) followed by brine (20 mL), dried over Na₂SO₄, and concentrated. The crude residue was purified by column chromatography (silica, 3% to 10% MeOH in DCM) to give benzyl 4-((2R)-2-methyl-1-(6-(4-(4-methyl-1-(1-methylpyrrolidin-3-yl)-1H-pyrazol-5-yl)piperidin-1-yl)-2-(trifluoromethyl)pyrimidin-4-yl)azetidin-3-yl)piperazine-1-carboxylate. m/z (ESI): 682.3 (M+H)⁺.

Step 6. To a solution of benzyl 4-((2R)-2-methyl-1-(6-(4-(4-methyl-1-(1-methylpyrrolidin-3-yl)-1H-pyrazol-5-yl)piperidin-1-yl)-2-(trifluoromethyl)pyrimidin-4-yl)azetidin-3-yl)piperazine-1-carboxylate (0.230 g, 0.337 mmol) in MeOH (10 mL) was added 10% palladium on carbon (18 mg, 0.17 mmol, Hindustan Platinum). The reaction mixture was stirred under hydrogen pressure (70 psi) for 16 h. The reaction mixture was cooled, filtered through celite bed, and washed with MeOH (100 mL). The filtrate was concentrated under reduced pressure to give 4-(4-(4-methyl-1-(1-methylpyrrolidin-3-yl)-1H-pyrazol-5-yl)piperidin-1-yl)-6-((2R)-2-methyl-3-(piperazin-1-yl)azetidin-1-yl)-2-(trifluoromethyl)pyrimidine. m/z (ESI): 548.4 (M+H)⁺.

Step 7. To a mixture of 4-(4-(4-methyl-1-(1-methylpyrrolidin-3-yl)-1H-pyrazol-5-yl)piperidin-1-yl)-6-((2R)-2-methyl-3-(piperazin-1-yl)azetidin-1-yl)-2-(trifluoromethyl)pyrimidine (0.12 g, 0.219 mmol) and Et₃N (0.092 mL, 0.657 mmol) in DCM (5 mL) was added acryloyl chloride (0.020 g, 0.219 mmol, Symax) at 0° C., and stirred for 0.5 h. The reaction mixture was quenched with ice cold water (15 mL) and extracted with DCM (2×15 mL). The organic combined organic extracts were washed with (15 mL), dried over Na₂SO₄, filtered, and concentrated. The crude residue was purified by Prep HPLC (X-select C18 (250×19 mm, 5.0 μm column) with a mobile phase of 0.1% ammonia in H₂O and ACN using a flow rate of 15 mL/min) to give 1-(4-((2R)-2-methyl-1-(6-(4-(4-methyl-1-(1-methylpyrrolidin-3-yl)-1H-pyrazol-5-yl)piperidin-1-yl)-2-(trifluoromethyl)pyrimidin-4-yl)azetidin-3-yl)piperazin-1-yl)prop-2-en-1-one. m/z (ESI): 602.3 (M+H)⁺. ¹H NMR (400 MHz, DMSO-d₆) δ ppm 7.15 (s, 1H), 6.80 (dd, 1H, J=16.7, 10.5 Hz), 6.11 (dd, 1H, J=16.6, 2.4 Hz), 5.69 (dd, 1H, J=10.5, 2.4 Hz), 5.62 (s, 1H), 4.97 (t, 1H, J=7.8 Hz), 4.41-4.56 (m, 3H), 3.91 (dt, 2H, J=33.0, 8.6 Hz), 3.58 (s, 4H), 3.23 (dd, 3H, J=17.3, 9.9 Hz), 2.90-3.07 (m, 3H), 2.65-2.78 (m, 2H), 2.57 (s, 1H), 2.28 (s, 7H), 2.16 (dq, 1H, J=11.7, 6.7, 6.2 Hz), 1.97 (s, 3H), 1.70-1.81 (m, 4H), 1.40 (d, 3H, J=6.3 Hz). ¹⁹F NMR (377 MHz, DMSO-d₆): δ ppm −70.22 (s, 1F).

### Example 2-137: 1-(4-((1S,3S)-3-(6-(4-(4-Methyl-1-(oxetan-3-yl)-1H-pyrazol-5-yl)piperidin-1-yl)-2-(trifluoromethyl)pyrimidin-4-yl)cyclobutyl)piperazin-1-yl)prop-2-en-1-one

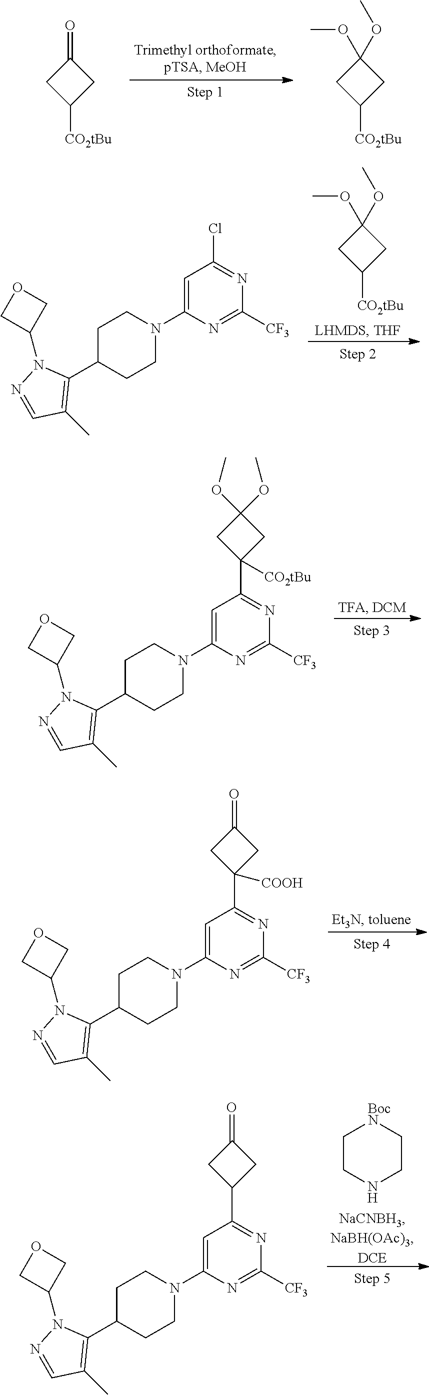

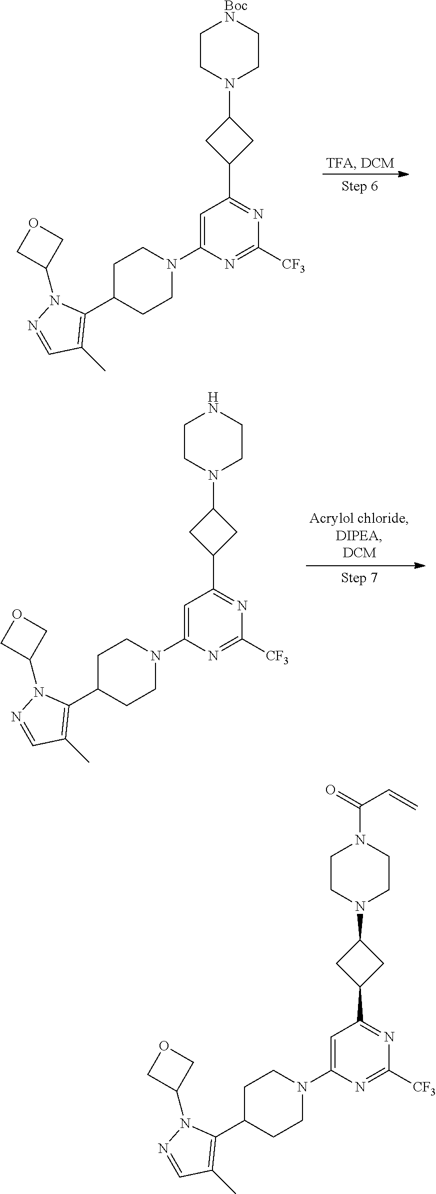

Step 1. A mixture of tert-butyl 3-oxocyclobutane-1-carboxylate (22 g, 129 Mmol, Combi-Blocks. Inc.), p-toluenesulfonic acid monohydrate (2.459 g, 12.93 mmol. Avra Synthesis), and trimethyl orthoformate (143 mL, 1293 mmol, Avra Synthesis) was taken in DCM (66 ml) and MeOH (66 mL) at rt. The solution was stirred for 2 h at rt. The solution was concentrated, diluted with diethyl ether (200 mL), extracted with satd. aq. NaHCO₃ (100 mL, and washed with brine (100 mL). The organic extract was dried over Na₂SO₄, filtered, and concentrated in vacuo to give tert-butyl 3,3-dimethoxycyclobutane-1-carboxylate, which was used without further purification. ¹H NMR (401 MHz, Chloroform-d) 6 ppm 3.17 (dd, 6H, J=6.5, 1.5 Hz), 2.7-2.9 (m, 1H), 2.3-2.5 (m, 4H), 1.46 (d, 9H, J=1.4 Hz).

Step 2. To a solution of ten-butyl 3,3-dimethoxycyclobutane-1-carboxylate (2.91 g, 13.44 mmol) in THF (36 mL) at 0° C. was added a solution of LiHMDS (generated by adding n-butyl lithium (8.40 mL, 13.44 mmol, Symax Industries) to an ice cooled solution of hexamethyldisilazane (2.82 mL, 13.44 mmol, TCl) in 5 mL THF) and the solution was stirred for 30 min. 4-Chloro-6-(4-(4-Methyl-1-(oxetan-3-yl)-1H-pyrazol-5-yl)piperidin-1-yl)-2-(trifluoromethyl)pyrimidine (1.8 g, 4.48 mmol) was added, wanned to rt and stirred for 16 h. The reaction mixture was quenched with satd. aq. NH₄Cl (10 mL) and extracted with EtOAc (3×20 mL). The organic extract was dried over Na₂SO₄, filtered. concentrated. The crude residue was purified by column chromatography (silica, 25 to 60% EtOAc:hexanes) to give tert-butyl 3,3-dimethoxy-1-(6-(4-(4-methyl-1-(oxetan-3-yl)-1H-pyrazol-5-yl)piperidin-1-yl)-2-(trifluoromethyl)pyrimidin-4-yl)cyclobutane-1-carboxylate. m/z (ESI): 582.3 (M+H)⁺.

¹H NMR (400 MHz, DMSO-d₆) δ ppm 7.30 (s, 1H), 6.95 (s, 1H), 5.76 (s, 1H), 5.71 (p, 1H, J=7.0 Hz), 4.8-5.0 (m, 4H), 4.60 (s, 1H), 2.9-3.2 (m, 9H), 2.6-2.8 (m, 4H), 1.99 (d, 3H, J=5.3 Hz), 1.76 (h, 4H, J=4.4, 3.5 Hz), 1.36 (s, 9H).

Step 3. tert-Butyl 3,3-dimethoxy-1-(6-(4-(4-methyl-1-(oxetan-3-yl)-1H-pyrazol-5-yl)piperidin-1-yl)-2-(trifluoromethyl)pyrimidin-4-yl)cyclobutane-1-carboxylate (2.3 g, 3.95 mmol) was taken in DCM (15 mL) at 0° C. TFA (6.09 mL, 79 mmol, Spectrochem) was added dropwise and the solution was stirred at rt for 16 h. The reaction mixture was concentrated under reduced pressure to give 1-(6-(4-(4-methyl-1-(oxetan-3-yl)-1H-pyrazol-5-yl)piperidin-1-yl)-2-(trifluoromethyl)pyrimidin-4-yl)-3-oxocyclobutane-1-carboxylic acid, which was used for next step without further purification. m/z (ESI): 480.1 (M+H)⁺.

Step 4. 1-(6-(4-(4-Methyl-1-(oxetan-3-yl)-1H-pyrazol-5-yl)piperidin-1-yl)-2-(trifluoromethyl)pyrimidin-4-yl)-3-oxocyclobutane-1-carboxylic acid (2 g, 4.17 mmol) was taken in toluene (10 mL) and triethylamine (2.91 mL, 20.86 mmol, Spectrochem) was added and stirred at 120° C. for 16 h. The reaction mixture was concentrated and the residue was purified by silica gel chromatography (40 to 50% EtOAc:hexane) to give 3-(6-(4-(4-methyl-1-(oxetan-3-yl)-1H-pyrazol-5-yl)piperidin-1-1)-2-(trifluoromethyl)pyrimidin-4-yl)cyclobutan-1-one. m/z (ESI): 435.7 (M+H)⁺. ¹H NMR (400 MHz, DMSO-d₆) δ ppm 7.30 (s, 1H), 7.15 (s, 1H), 5.71 (p, 1H, J=7.0 Hz), 4.8-5.0 (m, 3H), 4.62 (s, 1H), 3.6-3.8 (m, 2H), 3.3-3.5 (m, 3H), 3.11 (qd, 4H, J=7.3, 4.7 Hz), 1.99 (s, 2H), 1.76 (td, 3H, J=10.1, 9.0, 3.4 Hz), 1.39 (s, 1H), 1.18 (t, 2H, J=7.3 Hz).

Step 5. To a 50 mL round-bottomed flask was added 3-(6-(4-(4-methyl-1-(oxetan-3-yl)-1H-pyrazol-5-yl)piperidin-1-yl)-2-(trifluoromethyl)pyrimidin-4-yl)cyclobutan-1-one (0.750 g, 1.722 mmol) and 1-BOC-piperazine (0.642 g, 3.44 mmol, Spectrochem) in DCE (15 mL). Sodium triacetoxyborohydride (1.095 g, 5.17 mmol, Symax Laboratories) and sodium cyanoborohydride (0.216 g, 3.44 mmol, Sigma-Aldrich, Inc.) were added after cooling in an ice bath. The solution was warmed to rt and stirred for 16 h. The reaction mixture was cooled in an ice bath and quenched with said. aq. NaHCO₃ solution (10 mL) and extracted with EtOAc (3×20 mL). The combined organic extracts were dried over Na₂SO₄ and purified by column chromatography (50 to 80% EtOAc:hexanes) to give tert-butyl 4-(3-(6-(4-(4-methyl-1(oxetan-3-yl)-1H-pyrazol-5-yl)piperidin-1-yl)-2-(trifluoromethyl)pyrimidin-4-yl)cyclobutyl)piperazine-1-carboxylate m/z (ESI): 606.2 (M+H)⁺. ¹H NMR (400 MHz, DMSO-d₆) δ ppm 7.30 (s, 1H), 6.91 is, 1H), 5.70 (q, 1H, J=7.1 Hz), 4.8-5.0 (m, 4H), 4.60 (s, 2H), 3.91 (d, 1H, J=13.8 Hz), 2.8-3.1 (m, 2H), 2.6-2.8 (m, 1H), 2.4-2.5 (m, 1H), 2.34 (h, 2H, J=6.0, 4.3 Hz), 2.22 (t, 3H, J=5.1 Hz), 2.0-2.1 (m, 1H), 1.99 (s, 3H), 1.75 (td, 4H, J=10.3, 9.6, 3.4 Hz), 1.3-1.5 (m, 15H).

Step 6. To a round-bottomed flask was added tert-butyl 4-(3-(6-(4-(4-methyl-1-(oxetan-3-yl)-1H-pyrazol-5-yl)piperidin-1-yl)-2-(trifluoromethyl)pyrimidin-4-yl)cyclobutylpiperazine-1-carboxylate (0.8 g, 1.321 mmol) and TFA (0.102 mL, 1.321 mmol, Spectrochem) in DCM (16 mL). The solution was warmed to it and stirred for 3 h. The volatiles were removed in vacuo to give 4-(4-(4-methyl-1-(oxetan-3-yl)-1H-pyrazol-5-yl)piperidin-1-yl)-6-(3-(piperazin-1-yl)cyclobutyl)-2-(trifluoromethyl)pyrimidine trifluoroacetate salt, which was used as such in the next step. m/z (ESI): 506.3 (M+H)⁺.

Step 7. To a round-bottomed flask was added 4-(4-(4-methyl-1-(oxetan-3-yl)-1H-pyrazol-5-yl)piperidin-1-yl)-6-(3-(piperazin-1-yl)cyclobutyl)-2-(trifluoromethyl)pyrimidine trifluoroacetate salt (0.8 g, 1.582 mmol) and triethylamine (0.662 mL, 4.75 mmol, Spectrochem) in DCE (16 mL). Acryloyl chloride (0.143 g, 1.582 mmol, Symax Laboratories) was added while cooling in an ice bath. The solution was warmed to rt and stirred for 30 min. The reaction mixture was quenched with ice water (20 mL), extracted with DCM (3×10 mL), dried over Na₂SO₄, filtered, and concentrated. The residue was purified by preparative HPLC (X-select C18, 250.19 mm, 5.0 μm column with a mobile phase of 0.1% NH; in and ACN using a flow rate of 15 mL/min) to give 1-(4-(3-(6-(4-(4-methyl-1-(oxetan-3-yl)-1H-pyrazol-5-yl)piperidin-1-yl)-2-(trifluoromethyl)pyrimidin-4-yl)cyclobutyl)piperazin-1-yl)prop-2-en-1-one. m/z (ESI): 560.1 (M+H)⁺. ¹H NMR (401 MHz, DMSO-d₆) δ ppm 7.30 (s, 1H), 6.91 (s, 1H), 6.80 (dd, 1H, J=16.7, 10.4 Hz), 6.10 (dd, 1H, J=16.6, 2.5 Hz), 5.6-5.8 (m, 2H), 4.8-5.0 (m, 4H), 4.59 (s, 2H), 3.55 (dd, 4H, J=11.5, 5.4 Hz), 3.23-2.91 (m, 4H), 2.71 (ddd, 1H, J=9.0, 6.8, 2.2 Hz), 2.2-2.4 (m, 6H), 2.07 (qd, 2H, J=8.9, 2.5 Hz), 1.99 (s, 3H), 1.7-1.8 (m, 4H).

The examples in the table below were prepared in a manner similar to that described above.

| Ex. | Chemical Structure | Name | m/z (ESI): (M + H)⁺ |
|---|---|---|---|
| 2-138 | 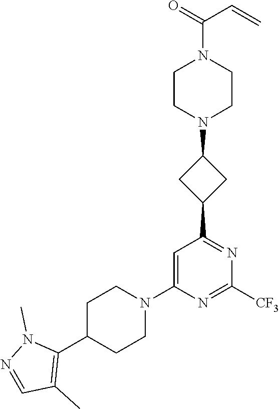 [SMILES: C=CC(=O)N1CCN([C@H]2C[C@@H](c3cc(N4CCC(c5c(C)cnn5C)CC4)nc(C(F)(F)F)n3)C2)CC1] | 1-(4-((1S,3S)-3-(6-(4-(1,4- dimethyl-1H-pyrazol-5- yl)piperidin-1-yl)-2- (trifluoromethyl)pyrimidin-4- yl)cyclobutyl)piperazin-1- yl)prop-2-en-1-one | 518.3 |
| 2-526 | 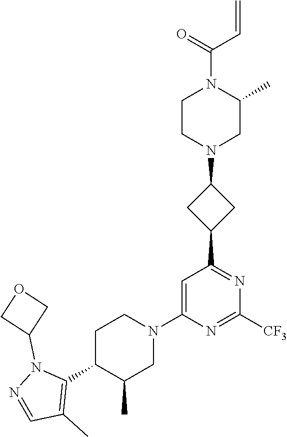 [SMILES: C=CC(=O)N1CCN([C@H]2C[C@@H](c3cc(N4CC[C@@H](c5c(C)cnn5C5COC5)[C@H](C)C4)nc(C(F)(F)F)n3)C2)C[C@H]1C] | 1-((2R)-2-methyl-4-(cis-3-(6- ((3S,4R)-3-methyl-4-(4-methyl- 1-(3-oxetanyl)-1H-pyrazol-5- yl)-1-piperidinyl)-2- (trifluoromethyl)-4- pyrimidinyl)cyclobutyl)-1- piperazinyl)-2-propen-1-one | 588.0 |
| 2-629 | 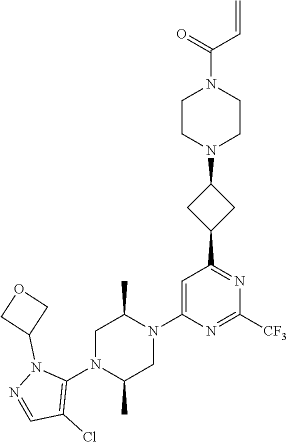 [SMILES: C=CC(=O)N1CCN([C@H]2C[C@@H](c3cc(N4C[C@@H](C)N(c5c(Cl)cnn5C5COC5)C[C@H]4C)nc(C(F)(F)F)n3)C2)CC1] | 1-(4-(cis-3-(6-((2R,5R)-4-(4- chloro-1-(3-oxetanyl)-1H- pyrazol-5-yl)-2.5-dimethyl-1- piperazinyl)-2-(trifluoromethyl)- 4-pyrimidinyl)cyclobutyl)-1- piperazinyl)-2-propen-1-one | 609.3 |
| 2-630 | 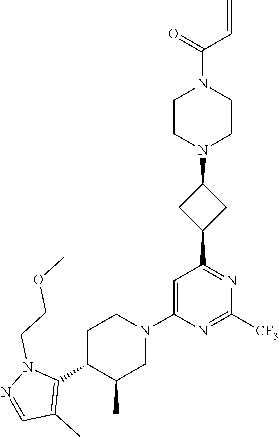 [SMILES: C=CC(=O)N1CCN([C@H]2C[C@@H](c3cc(N4CC[C@@H](c5c(C)cnn5CCOC)[C@H](C)C4)nc(C(F)(F)F)n3)C2)CC1] | 1-(4-(cis-3-(6-((3S,4R)-4-(1-(2- methoxyethyl)-4-methyl-1H- pyrazol-5-yl)-3-methyl-1- piperidinyl)-2-(trifluoromethyl)- 4-pyrimidinyl)cyclobutyl)-1- piperazinyl)-2-propen-1-one | 576.0 |
| 2-631 | 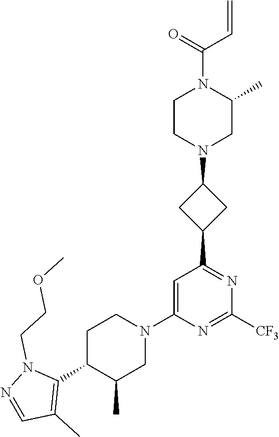 [SMILES: C=CC(=O)N1CCN([C@H]2C[C@@H](c3cc(N4CC[C@@H](c5c(C)cnn5CCOC)[C@H](C)C4)nc(C(F)(F)F)n3)C2)C[C@H]1C] | 1-((2R)-4-(cis-3-(6-((3S,4R)-4- (1-(2-methoxyethyl)-4-methyl- 1H-pyrazol-5-yl)-3-methyl-1- piperidinyl)-2-(trifluoromethyl)- 4-pyrimidinyl)cyclobutyl)-2- methyl-1-piperazinyl)-2-propen- 1-one | 590.1 |
| 2-149 | 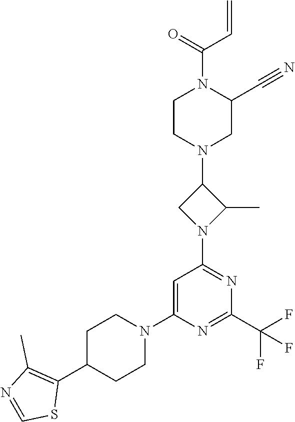 [SMILES: C=CC(=O)N1CCN(C2CN(c3cc(N4CCC(c5scnc5C)CC4)nc(C(F)(F)F)n3)C2C)CC1C#N] | 4-(2-methyl-1-(6-(4-(4-methyl- 1,3-thiazol-5-yl)-1-piperidinyl)- 2-(trifluoromethyl)-4- pyrimidinyl)-3-azetidinyl)-1-(2- propenoyl)-2- piperazinecarbonitrile | 603.2 |
| 2-150 | 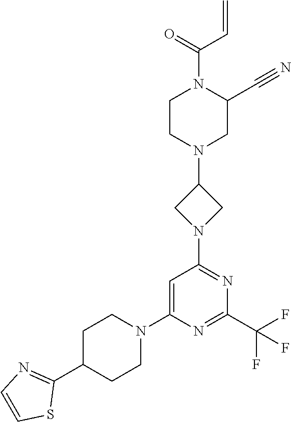 [SMILES: C=CC(=O)N1CCN(C2CN(c3cc(N4CCC(c5nccs5)CC4)nc(C(F)(F)F)n3)C2)CC1C#N] | 1-(2-propenoyl)-4-(1-(6-(4-(1,3- thiazol-2-yl)-1-piperidinyl)-2- (trifluoromethyl)-4- pyrimidinyl)-3-azetidinyl)-2- piperazinecarbonitrile | 540.2 |
| 2-335 | 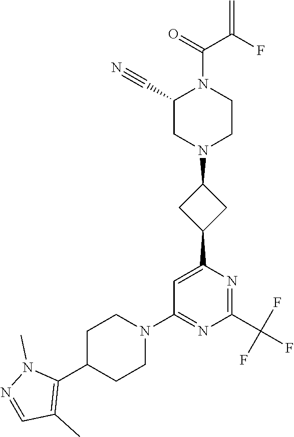 [SMILES: C=C(F)C(=O)N1CCN([C@H]2C[C@@H](c3cc(N4CCC(c5c(C)cnn5C)CC4)nc(C(F)(F)F)n3)C2)C[C@@H]1C#N] | (2R)-4-(cis-3-(6-(4-(1,4- dimethyl-1H-pyrazol-5-yl)-1- piperidinyl)-2-(trifluoromethyl)- 4-pyrimidinyl)cyclobutyl)-1-(2- fluoro-2-propenoyl)-2- piperazinecarbonitrile | 561.2 |
| 2-367 | 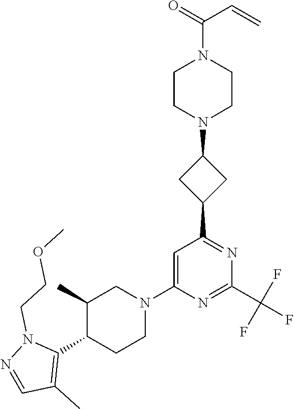 [SMILES: C=CC(=O)N1CCN([C@H]2C[C@@H](c3cc(N4CC[C@H](c5c(C)cnn5CCOC)[C@@H](C)C4)nc(C(F)(F)F)n3)C2)CC1] | 1-(4-(cis-3-(6-((3R,4S)-4-(1-(2- methoxyethyl)-4-methyl-1H- pyrazol-5-yl)-3-methyl-1- piperidinyl)-2-(trifluoromethyl)- 4-pyrimidinyl)cyclobutyl)-1- piperazinyl)-2-propen-1-one | 576.4 |
| | mixture of trans isomers | | |
| | at piperidine | | |
| 2-357 | 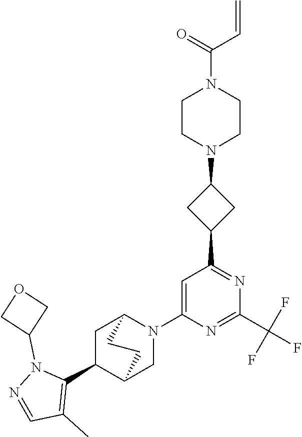 [SMILES: C=CC(=O)N1CCN([C@H]2C[C@@H](c3cc(N4C[C@@H]5CC[C@H]4C[C@@H]5c4c(C)cnn4C4COC4)nc(C(F)(F)F)n3)C2)CC1] | 1-(4-(cis-3-(6-((1S,4R,5S)-5-(4- methyl-1-(3-oxetanyl)-1H- pyrazol-5-yl)-2- azabicyclo[2.2.2]octan-2-yl)-2- (trifluoromethyl)-4- pyrimidinyl)cyclobutyl)-1- piperazinyl)-2-propen-1-one | 586.2 |
| 2-306 | 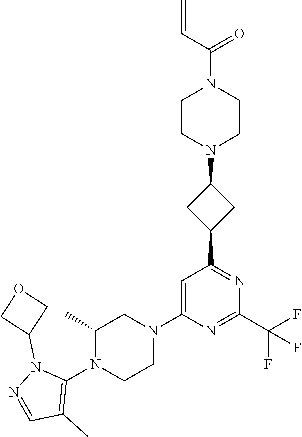 [SMILES: C=CC(=O)N1CCN([C@H]2C[C@@H](c3cc(N4CCN(c5c(C)cnn5C5COC5)[C@H](C)C4)nc(C(F)(F)F)n3)C2)CC1] | 1-(4-(cis-3-(6-((3R)-3-methyl-4- (4-methyl-1-(3-oxctany])-1H- pyrazol-5-yl)-1-piperazinyl)-2- (trifluoromethyl)-4- pyrimidinyl)cyclobutyl)-1- piperazinyl)-2-propen-1-one | 575.1 |
| 2-307 | 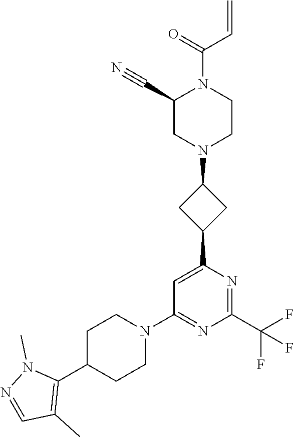 [SMILES: C=CC(=O)N1CCN([C@H]2C[C@@H](c3cc(N4CCC(c5c(C)cnn5C)CC4)nc(C(F)(F)F)n3)C2)C[C@H]1C#N] | (2S)-4-(cis-3-(6-(4-(1,4- dimethyl-1H-pyrazol-5-yl)-1- piperidinyl)-2-(trifluoromethyl)- 4-pyrimidinyl)cyclobutyl)-1-(2- propenoyl)-2- piperazinecarbonitrile | 543.2 |
| 2-308 | 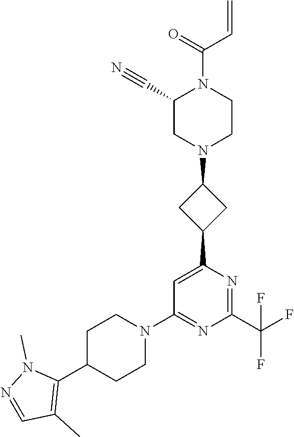 [SMILES: C=CC(=O)N1CCN([C@H]2C[C@@H](c3cc(N4CCC(c5c(C)cnn5C)CC4)nc(C(F)(F)F)n3)C2)C[C@@H]1C#N] | (2R)-4-(cis-3-(6-(4-(1,4- dimethyl-1H-pyrazol-5-yl)-1- piperidinyl)-2-(trifluoromethyl)- 4-pyrimidinyl)cyclobutyl)-1-(2- propenoyl)-2- piperazinecarbonitrile | 543.2 |
| 2-309 | 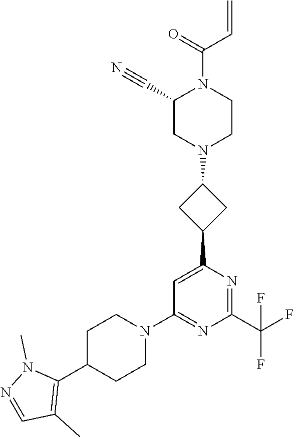 [SMILES: C=CC(=O)N1CCN([C@H]2C[C@H](c3cc(N4CCC(c5c(C)cnn5C)CC4)nc(C(F)(F)F)n3)C2)C[C@@H]1C#N] | (2R)-4-(trans-3-(6-(4-(1,4- dimethyl-1H-pyrazol-5-yl)-1- piperidinyl)-2-(trifluoromethyl)- 4-pyrimidinyl)cyclobutyl)-1-(2- propenoyl)-2- piperazinecarbonitrile | 543.2 |
| 2-337 | 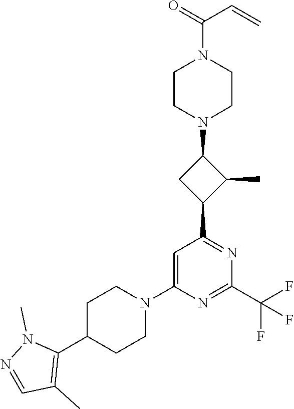 [SMILES: C=CC(=O)N1CCN([C@@H]2C[C@H](c3cc(N4CCC(c5c(C)cnn5C)CC4)nc(C(F)(F)F)n3)[C@@H]2C)CC1] | 1-(4-((1R,2S,3S)-3-(6-(4-(1,4- dimethyl-1H-pyrazol-5-yl)-1- piperidinyl)-2-(trifluoromethyl)- 4-pyrimidinyl)-2- methylcyclobutyl)-1- piperazinyl)-2-propen-1-one | 532.0 |
| | mixture of all cis isomers | | |
| | at cyclobutanc | | |
| 2-350 | 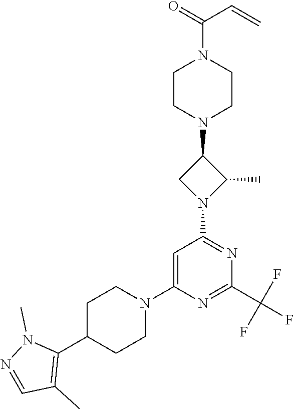 | 1-(4-((1R,2R,3R)-3-(6-(4-(1,4- dimethyl-1H-pyrazol-5-yl)-1- piperidinyl)-2-(trifluoromethyl)- 4-pyrimidinyl)-2- methylcyclobutyl)-1 - piperazinyl)-2-propen-1-one | 532.3 |
| 2-172 | 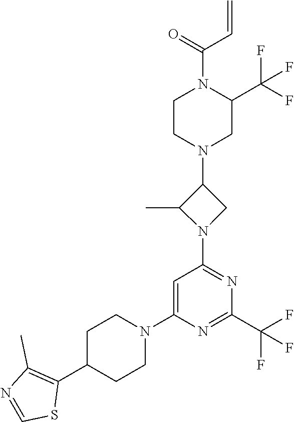 [SMILES: C=CC(=O)N1CCN(C2CN(c3cc(N4CCC(c5scnc5C)CC4)nc(C(F)(F)F)n3)C2C)CC1C(F)(F)F] | 1-(4-(2-methy 1-1-(6-(4-(4- methyl-1,3-thiazol-5-yl)-1- piperidinyl)-2-(trifluoromethyl)- 4-pyrimidinyl)-3-azetidinyl)-2- (trifluoromethyl)-1-piperazinyl)- 2-propen-1-one | 603.2 |
| 2-174 | 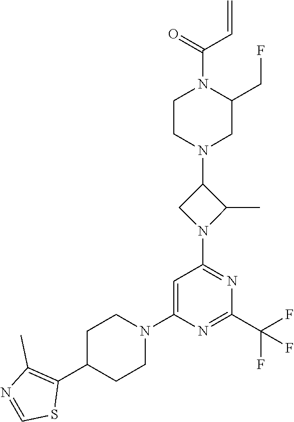 [SMILES: C=CC(=O)N1CCN(C2CN(c3cc(N4CCC(c5scnc5C)CC4)nc(C(F)(F)F)n3)C2C)CC1CF] | 1-(2-(fluoromethyl)-4-(2- methyl-1-(6-(4-(4-methyl-1,3- thiazol-5-yl)-1-piperidinyl)-2- (trifluoromethyl)-4- pyrimidinyl)-3-azetidinyl)-1- piperazinyl)-2-propen-1-one | 567.2 |

### Example 2-139: 1-(4-(1-(6-(4-(1,4-Dimethyl-1H-pyrazol-5-yl)piperidin-1-yl)-2-(trifluoromethyl)pyrimidin-4-yl)-2-(hydroxymethyl)azetidin-3-yl)piperazin-1-yl)prop-2-en-1-one

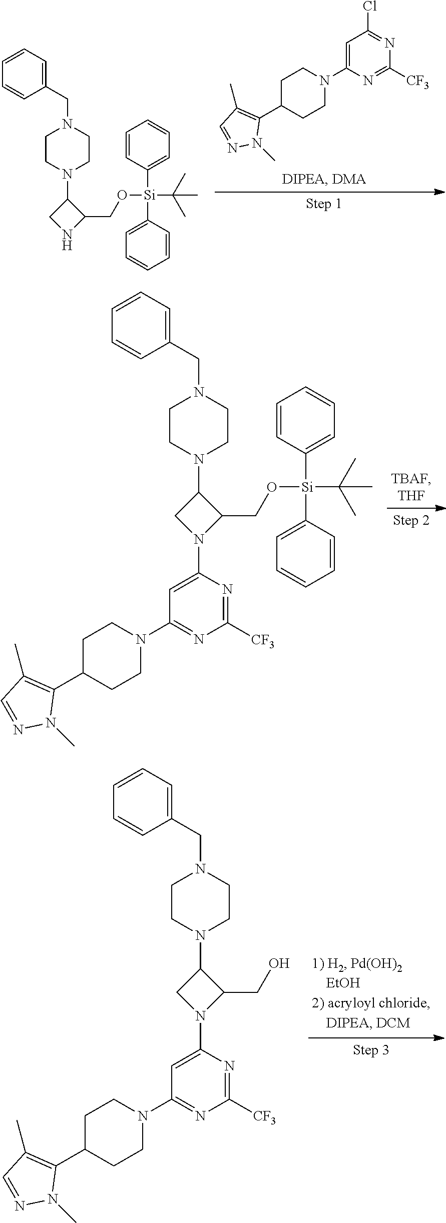

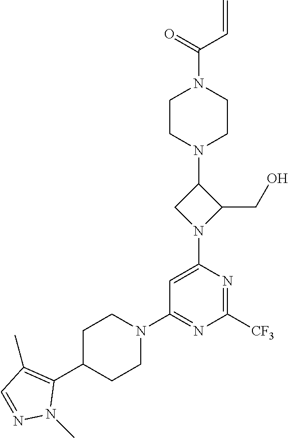 [SMILES: C=CC(=O)N1CCN(C2CN(c3cc(N4CCC(c5c(C)cnn5C)CC4)nc(C(F)(F)F)n3)C2CO)CC1]

Step 1. A glass vial was charged with 1-benzyl-4-(2-(((tert-butyldiphenylsilyl)oxy)methyl)azetidin-3-yl)piperazine (0.150 g, 0.300 mmol) and 4-chloro-6-(4-(1,4-dimethyl-1H-pyrazol-5-yl)piperidin-1-yl)-2-(trifluoromethyl)pyrimidine (0.108 g, 0.300 mmol) in DMA (1.5 mL). Then DIPEA (0.16 mL, 0.900 mmol, Sigma-Aldrich, Inc.) was added and the reaction mixture was stirred at 80° C. for 16 h. The reaction mixture was diluted with H₂O and DCM, stirred 30 min. and the extracts were separated. The aqueous layer was extracted with DCM (3×). The combined organic extracts were dried over MgSO₄, filtered, and concentrated in vacuo. The crude material was purified by silica gel chromatography, eluting with a gradient of (W % to 50% EtOAc:EtOH (3:1) in heptane, to provide 4-(3-(4-benzylpiperazin-1-yl)-2-(((tert-butyldiphenylsilyl)oxy)methyl)azetidin-1-yl)-6-(4-(1,4-dimethyl-1H-pyrazol-5-yl)piperidin-1-yl)-2-(trifluoromethyl)pyrimidine. m/z (ESI): 823.3 (M+H)⁺.

Step 2. To a 50 mL round-bottomed flask was added 4-(3-(4-benzylpiperazin-1-yl)-2-(((tert-butyldiphenylsilyl)oxy)methyl)azetidin-1-yl)-6-(4-(1,4-dimethyl-1H-pyrazol-5-yl)piperidin-1-yl)-2-(trifluoromethyl)pyrimidine (0.200 g, 0.243 mmol) and THF (2.4 mL). The reaction mixture was cooled to 0° C. with an ice bath, while stirred under an inert (N₂) atmosphere. Then, tetrabutylammonium fluoride (1.0 M in THF, 1.2 mL, 1.21 mmol, Sigma-Aldrich, Inc.) was added dropwise to the reaction mixture. The ice bath was removed and the reaction mixture was allowed to stir for 3 h. The mixture was quenched with sat. aq. NH₄Cl, then diluted with H₂O. The extracts were separated and the aqueous layer was extracted with EtOAc (3×). The combined organic extracts were dried over MgSO₄, filtered, and concentrated in vacuo. The crude material was absorbed onto a plug of silica gel and purified by chromatography through a Redi-Sep pre-packed silica gel column, eluting with a gradient of 0% to 30% 2 M NH₃-MeOH in DCM, to provide (3-(4-benzylpiperazin-1-yl)-1-(6-(4-(1,4-dimethyl-1H-pyrazol-5-yl)piperidin-1-yl)-2-(trifluoromethyl)pyrimidin-4-yl)azetidin-2-yl)methanol. m/z (ESI): 585.2 (M+H)⁺.

Step 3. To a 50 mL glass tube was added (3-(4-benzylpiperazin-1-yl)-1-(6-(4-(1,4-dimethyl-1H-pyrazol-5-yl)piperidin-1-yl)-2-(trifluoromethyl)pyrimidin-4-yl)azetidin-2-yl)methanol (0.142 g, 0.243 mmol) and palladium hydroxide on activated charcoal (50/o H₂O) (0.085 g, 0.121 mmol, Combi-Blocks, Inc.) in EtOH (2.5 mL), while under a stream of Argon (gas). The reaction mixture atmosphere was evacuated, then backfilled with hydrogen (gas) 3 times. Then the reaction mixture was placed under hydrogenation conditions at 40 psi for 24 h. The reaction mixture was filtered through a pad of Celite and the filter cake was rinsed with NH₃ in MeOH (2N). The filtrate was concentrated and the crude mixture was used in the next step of the synthesis, without further purification. m/z (ESI): 495.2 (M+H)⁺.

To a 50 mL round-bottomed flask was added (1-(6-(4-(1,4-dimethyl-1H-pyrazol-5-yl)piperidin-1-yl)-2-(trifluoromethyl)pyrimidin-4-yl)-3-(piperazin-1-yl)azetidin-2-yl)methanol (0.120 g, 0.243 mmol) and DIPEA (0.094 g, 0.2 mL, 0.728 mmol, Sigma-Aldrich, Inc.) in DCM (1.2 mL). The mixture was cooled to 0° C., then a solution of acryloyl chloride (0.2 M in DCM) (1.2 mL, 0.243 mmol, Sigma-Aldrich, Inc.) was added slowly to the mixture. The overall mixture was stirred at 0° C. for 5 mm, then warmed to rt over 30 min. The reaction mixture was quenched with sat. aq. NaHCO₃, the extracts were separated, and the aqueous layer was extracted with DCM (3×). The combined organic extracts were dried over MgSO₄, filtered, and concentrated in vacuo. The crude material was absorbed onto a Biotage C18 column and purified by reverse-phase chromatography, eluting with a gradient of 5% to 45% MeCN (TFA 0.1%) in H₂O (TFA 0.1%), to provide 1-(4-(1(6-(4-(1,4-dimethyl-1H-pyrazol-5-yl)piperidin-1-yl)-2-(trifluoromethylpyrimidin-4-yl)-2-(hydroxymethyl)azetidin-3-ylpiperazin-1-yl)prop-2-en-1-one. m/z (ESI): 549.1 (M+H)⁺.

### SFC Conditions for Chiral Separation

| | | | Racemic SM \| |
|---|---|---|---|
| Ex. # | Chemical Structure | Name | separation condition |
| 2-139-1 | 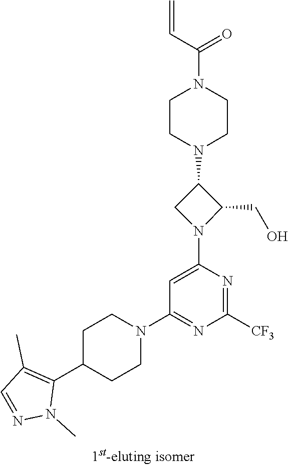 [SMILES: C=CC(=O)N1CCN([C@H]2CN(c3cc(N4CCC(c5c(C)cnn5C)CC4)nc(C(F)(F)F)n3)[C@H]2CO)CC1] | 1-(4-((2R.3S)-1-(6-(4-(1,4- dimethyl-1H-pyrazol-5- yl)piperidin-1-yl)-2- (trifluoromethyl)pyrimidin-4-yl)- 2-(hydroxymethyl)azetidin-3- yl)piperazin-1-yl)prop-2-en-1- one | 2-139 \| SFC (Chiralpak IC, 21 × 150 mm 5 μm, column with a mobile phase of 50% MeOH with 0.2% TEA using a flow rate of 80 mL/min) |
| 2-139-2 | 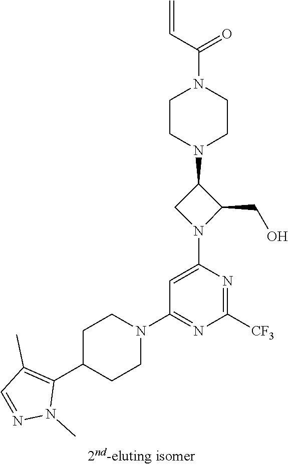 [SMILES: C=CC(=O)N1CCN([C@@H]2CN(c3cc(N4CCC(c5c(C)cnn5C)CC4)nc(C(F)(F)F)n3)[C@@H]2CO)CC1] | 1-(4-((2S,3R)-1-(6-(4-(1,4- dimethyl-1H-pyrazol-5- yl)piperidin-1-yl)-2- (trifluoromethyl)pyrimidin-4-yl)- 2-(hydroxymethyl)azetidin-3- yl)piperazin-1-yl)prop-2-en-1- one | 2-139 \| SFC (Chiralpak IC, 21 × 150 mm 5 μm, column with a mobile phase of 50% MeOH with 0.2% TEA using a flow rate of 80 mL/min) |

### Example 2-140: 1-(4-(1-(6-(4-(1,4-Dimethyl-1H-pyrazol-5-yl)piperidin-1-yl)-2-(trifluoromethyl)pyrimidin-4-yl)-2-(fluoromethyl)azetidin-3-yl)piperazin-1-yl)prop-2-en-1-one

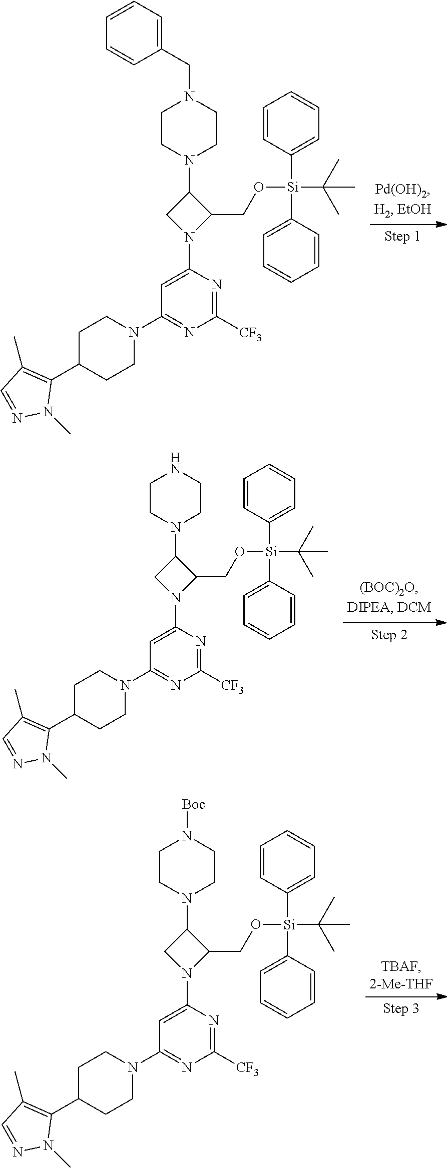 [SMILES: Cc1cnn(C)c1C1CCN(c2cc(N3CC(N4CCN(Cc5ccccc5)CC4)C3CO[Si](c3ccccc3)(c3ccccc3)C(C)(C)C)nc(C(F)(F)F)n2)CC1 ; Cc1cnn(C)c1C1CCN(c2cc(N3CC(N4CCNCC4)C3CO[Si](c3ccccc3)(c3ccccc3)C(C)(C)C)nc(C(F)(F)F)n2)CC1 ; Cc1cnn(C)c1C1CCN(c2cc(N3CC(N4CCN(C(=O)OC(C)(C)C)CC4)C3CO[Si](c3ccccc3)(c3ccccc3)C(C)(C)C)nc(C(F)(F)F)n2)CC1]

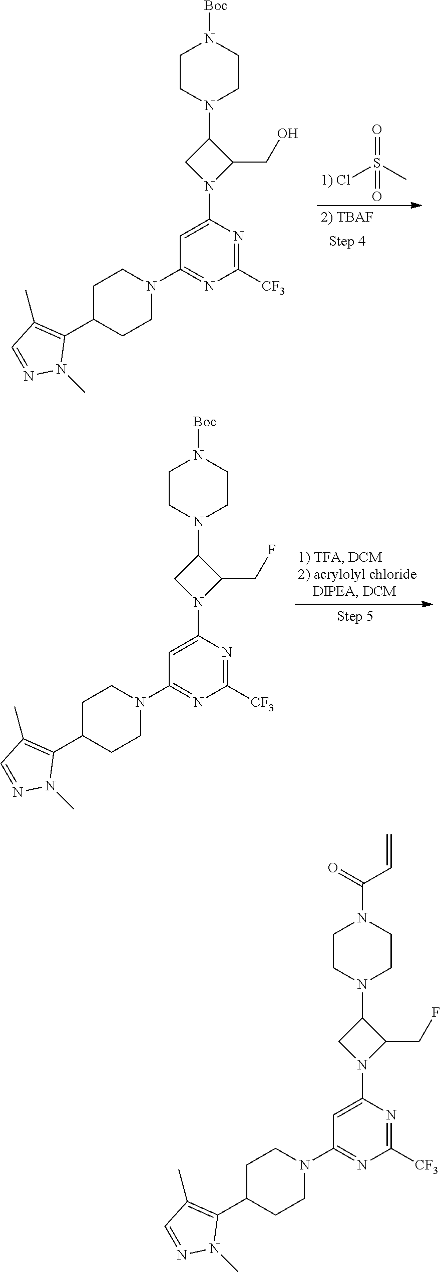

Step 1. To a 50 mL glass tube was added 4-(3-(4-benzylpiperazin-1-yl)-2-(((tert-butyldiphenylsilyloxy)methyl)azetidin-1-yl)-6-(4-(1,4-dimethyl-1H-pyrazol-5-yl)piperidin-1-yl)-2-(trifluoromethyl)pyrimidine (1.33 g, 1.62 mmol) and 20% palladium hydroxide on activated charcoal (0.567 g, 0.808 mmol, Combi-Blocks, Inc.) in EtOH (22 mL)/MeOH (11 mL), while under a stream of argon (gas). The reaction mixture atmosphere was evacuated, then backfilled with hydrogen (gas) 3 times. Then the reaction mixture was placed under hydrogenation conditions at 40 psi for 32 h. The reaction mixture was filtered through a pad of Celite and the filter cake was rinsed with NH₃ in MeOH (2N). The filtrate was concentrated in vacuo and the crude mixture was taken to the next step without further purification. m/z (ESI): 733.3 (M+H)⁺.

Step 2. To a 150 mL round-bottomed flask was added 4-(2-(((tert-butyldiphenylsilyl)oxy)methyl)-3-(piperazin-1-yl)azetidin-1-yl)-6-1⁴41,4-dimethyl-11H-pyrazol-5-yl)piperidin-1-yl)-2-(trifluoromethyl)pyrimidine (0.570 g, 0.778 mmol) and DIPEA (0.4 mL, 2.33 mmol, Sigma-Aldrich, Inc.) in DCM (4 mL). The mixture was cooled to 0° C., then di-tert-butyl dicarbonate (0.204 g, 0.933 mmol, Oakwood Products, Inc.) was added to the reaction mixture and stirred 2 h. The reaction mixture was quenched with sat. aq. NaHCO₃. The extracts were separated and the aqueous layer was extracted with DCM (3×). The combined organic extracts were dried over MgSO₄, filtered, and concentrated in vacuo. The crude material was absorbed onto a plug of silica gel and purified by chromatography through a Redi-Sep pre-packed silica gel column, eluting with a gradient of 0% to 50% EtOAc/EtOH (3:1) in heptane, to provide tert-butyl 4-(2-(((tert-butyldiphenylsilyl)oxy)methyl)-1-(6-(4-(1,4-dimethyl-1H-pyrazol-5-yl)piperidin-1-yl)-2-(trifluoromethyl)pyrimidin-4-yl)azetidin-3-yl)piperazine-1-carboxylate. ¹H NMR (400 MHz, DMSO-d₆) δ ppm 7.68 (d, J=7.4 Hz, 2H), 7.55 (br d, J=6.9 Hz, 2H), 7.35-7.49 (m, 6H), 7.06 (s, 1H), 5.67 (s, 1H), 4.46-4.57 (m, 1H), 4.38 (br d, J=13.4 Hz, 2H), 4.22 (br dd, J=10.7, 6.1 Hz, 1H), 3.89-4.0 (m, 3H), 3.75 (s, 3H), 3.33-3.42 (m, 1H), 3.22-3.28 (m, 3H), 3.08 (br t, J=11.8 Hz, 1H), 2.81-2.98 (m, 2H), 2.31-2.48 (m, 3H), 2.26 (br d, J=5.2 Hz, 2H), 1.92 (s, 3H), 1.63-1.79 (m, 4H), 1.34-1.44 (m, 9H), 0.91-1.02 (m, 9H). m/z (ESI): 834.3 (M+H)⁺.

Step 3. To a 100 mL round-bottomed flask was added tert-butyl 4-(2-(((tert-butyldiphenylsilyl)oxy)methyl)-1-(6-(4-(1,4-dimethyl-1H-pyrazol-5-yl)piperidin-1-yl)-2-(trifluoromethyl)pyrimidin-4-yl)azetidin-3-yl)piperazine-1-carboxylate (0.400 g, 0.480 mmol) in 2-methyltetrahydrofuran (5 mL). The reaction mixture was cooled to 0° C. with an ice bath, while stirred under an inert (N₂) atmosphere. Then TBAF, 1.0 M in THF (2.4 mL, 2.4 mmol, Sigma-Aldrich. Inc.) was added dropwise to the reaction mixture. The ice bath was removed and the reaction mixture was allowed to stir for 16 h. The mixture was quenched with sat aq. NH₄Cl, then the mixture was diluted with DCM and H₂O. The extracts were separated and the aqueous layer was extracted with DCM (3×). The combined organic extracts were dried over MgSO₄, filtered, and concentrated in vacuo. The crude material was purified by chromatography through an ISCO silica-gel column, eluting with a gradient of 0% to 100% EtOAc/EtOH (3:1) in heptane, to provide tert-butyl 4-(1-(6-(4-(1,4-dimethyl-1H-pyrazol-5-yl)piperidin-1-yl)-2-(trifluoromethyl)pyrimidin-4-yl)-2-(hydroxymethyl)azetidin-3-yl)piperazine-1-carboxylate ¹H NMR (400 MHz, DMSO-d₆) δ ppm 7.07 (s, 1H), 5.76 (br s, 1H), 4.88 (br s, 1H), 4.47 (br d, J=11.8 Hz, 2H), 4.32-4.40 (m, 1H), 3.75-4.01 (m, 7H), 3.36-3.41 (m, 2H), 3.30-3.34 (m, 1H), 3.04-3.18 (m, 1H), 2.94 (br t, J=11.5 Hz, 2H), 2.40 (br s, 2H), 2.22-2.35 (m, 2H), 1.98 (s, 3H), 1.70-1.83 (m, 4H), 1.40 (s, 9H), 1.21-1.36 (m, 2H). m/z (ESI): 595.3 (M+H)⁺.

Step 4. To a 50 mL round-bottomed flask was added tert-butyl 4-(1(6-(4-(1,4-dimethyl-1H-pyrazol-5-yl)piperidin-1-yl)-2-(trifluoromethyl)pyrimidin-4-yl)-2-(hydroxymethyl)azetidin-3-yl)piperazine-1-carboxylate (0.110 g, 0.185 mmol) and triethylamine (0.037 g, 0.1 mL, 0.370 mmol, Sigma-Aldrich, Inc.) in DCM (4 mL). The mixture was cooled to 0° C., then treated dropwise with methanesulfonyl chloride (0.02 mL, 0.277 mmol, Sigma-Aldrich. Inc.), stirred at rt for 30 mm. The mixture was treated with aq. NaHCO₃ (5 mL) and the extracts were separated. The aqueous layer was extracted with DCM (3×), the combined organic extracts were dried (MgSO₄), filtered, concentrated in vacuo. The crude material was absorbed onto a plug of silica gel and purified by chromatography through an Interchim (15 micron) silica-gel column, eluting with a gradient of 0% to 60% EtOAc/EtOH (3:1) in heptane, to provide tert-butyl 4-(1-(6-(4-(1,4-dimethyl-1H-pyrazol-5-yl)piperidin-1-yl)-2-(trifluoromethyl)pyrimidin-4-yl)-2-(((methylsulfonyl)oxy))methyl)azetidin-3-yl)piperazine-1-carboxylate m/z (ESI): 673.3 (M+H)⁺. The previous residue was diluted with THF (4 mL), then treated with TBAF (1.0 M in THF) (1.7 mL, 1.7 mmol, Sigma-Aldrich, Inc.) and heated under reflux for 2 h. The reaction mixture was cooled to 0° C., then treated with H₂O. The mixture was diluted with EtOAc and the aqueous layer was extracted with EtOAc (3×). The organic extracts were washed with NaHCO₃ solution, washed with brine, dried (MgSO₄), filtered, and concentrated in vacuo. The crude residue was used without further purification. m/z (ESI): 597.2 (M+H)⁺.

Step 5. To a 50 mL round-bottomed flask was added ter-butyl 4-(1-(6-(4-(1,4-dimethyl-1H-pyrazol-5-yl)piperidin-1-yl)-2-(trifluoromethyl)pyrimidin-4-yl)-2-(fluoromethyl)azetidin-3-yl)piperazine-1-carboxylate (0.100 g, 0.168 mmol) and TFA (0.2 mL, 1.68 mmol, Apollo Scientific Ltd.) in DCM (2 mL). The reaction mixture was stirred at rt for 4 h and concentrated in vacuo. The crude residue was used without further purification. m/z (ESI): 497.3 (M+H)⁺. The previous residue was dissolved in DCM (2 mL) and the mixture was cooled to 0° C. Then DIPEA (0.5 mL, 2.51 mmol, Sigma-Aldrich, Inc.), followed by acryloyl chloride (0.2 M in DCM) (0.9 mL, 0.168 mmol, Sigma-Aldrich, Inc.) were added to the reaction mixture. The overall mixture was stirred and warmed to rt over 15 min. The reaction mixture was diluted with DCM and H₂O, then quenched with sat aq. NaHCO₃ and the extracts were separated. The aqueous layer was extracted with DCM (3×), then the combined organic extracts were dried over MgSO₄, filtered, and concentrated in vacuo. The crude residue was purified by Biotage Reverse-phase C18 chromatography, eluting with a gradient of 10% to 25% MeCN (TFA 0.1%) in H₂O (TFA 0.1%), then with a gradient of 25-40% MeCN (TFA 0.1%) in H₂O (TFA 0.1%), to afford 1-(4-(1-(6-(4-(1,4-dimethyl-1H-pyrazol-5-yl)piperidin-1-yl)-2-(trifluoromethyl)pyrimidin-4-yl)-2-(fluoromethyl)azetidin-3-yl)piperazin-1-yl)prop-2-en-1-one. m/z (ESI): 551.2 (M+H)⁺.

Step 6. The sample was purified via SFC using a Chiralpak IC, 21×150 mm 5 μm, column with a mobile phase of 55% MeOH using a flow rate of 80 mL/min to provide (peak 2) 1-(4-((2S,3R)-1-(6-(4-(1,4-dimethyl-1H-pyrazol-5-yl)piperidin-1-yl)-2-(trifluoromethyl)pyrimidin-4-yl)-2-(fluoromethyl)azetidin-3-yl)piperazin-1-yl)prop-2-en-1-one. ¹H NMR (400 MHz, chloroform-d) δ ppm 7.21 (s, 1H), 6.58 (dd, J=16.8, 10.6 Hz, 1H), 6.32 (dd, J=16.8, 1.7 Hz, 1H), 5.74 (dd, J=10.6, 1.9 Hz, 1H), 5.42 (s, 1H), 4.94-5.14 (m, 1H), 4.73-4.91 (m, 1H), 4.60-4.70 (m, 1H), 4.55 (br d, J=12.4 Hz, 2H), 3.98-4.10 (m, 2H), 3.86 (s, 3H), 3.58-3.76 (m, 3H), 3.48 (q, J=7.2 Hz, 1H), 2.86-3.01 (m, 3H), 2.54 (br s, 1H), 2.45 (br s, 2H), 2.39 (br s, 1H), 2.07 (s, 3H), 1.81-2.01 (m, 5H). m/z (ESI): 551.3 (M+H)⁺.

### SFC Conditions for Chiral Separation

| | | | Racemic SM |
|---|---|---|---|
| Ex. # | Chemical Structure | Name | separation condition |
| 2-140-1 | 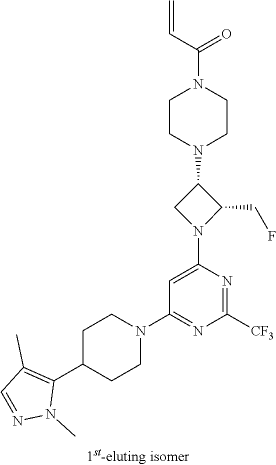 [SMILES: C=CC(=O)N1CCN([C@H]2CN(c3cc(N4CCC(c5c(C)cnn5C)CC4)nc(C(F)(F)F)n3)[C@H]2CF)CC1] | 1-(4-((2R,3S)-1-(6-(4-(1,4- dimethyl-1H-pyrazol-5- yl)piperidin-1-yl)-2- (trifluoromethyl)pyrimidin-4-yl)- 2-(fluoromethyl)azetidin-3- yl)piperazin-1-yl)prop-2-en-1- one | 2-140 \| SFC (Chiralpak IC, 21 × 150 mm 5 μm, column with a mobile phase of 55% MeOH using a flow rate of 80 mL/min) |
| 2-140-2 | 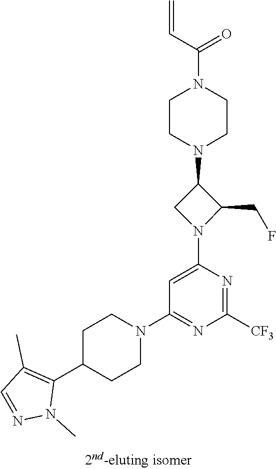 [SMILES: C=CC(=O)N1CCN([C@@H]2CN(c3cc(N4CCC(c5c(C)cnn5C)CC4)nc(C(F)(F)F)n3)[C@@H]2CF)CC1] | 1-(4-((2S,3R)-1-(6-(4-(1,4- dimethyl-1H-pyrazol-5- yl)piperidin-1-yl)-2- (trifluoromethyl)pyrimidin-4-yl)- 2-(fluoromethyl)azetidin-3- yl)piperazin-1-yl)prop-2-en-1- one | 2-140 \| SFC (Chiralpak IC, 21 × 150 mm 5 μm, column with a mobile phase of 55% MeOH using a flow rate of 80 mL/min) |

### Example 2-353: 1-(4-((2R,3R)-1-(6-(4-(2-(3-methoxyoxetan-3-yl)-4-methylpyridin-3-yl)piperidin-1-yl)-2-(trifluoromethyl)pyrimidin-4-yl)-2-methyl)azetidin-3-yl)piperazin-1-yl)prop-2-en-1-one

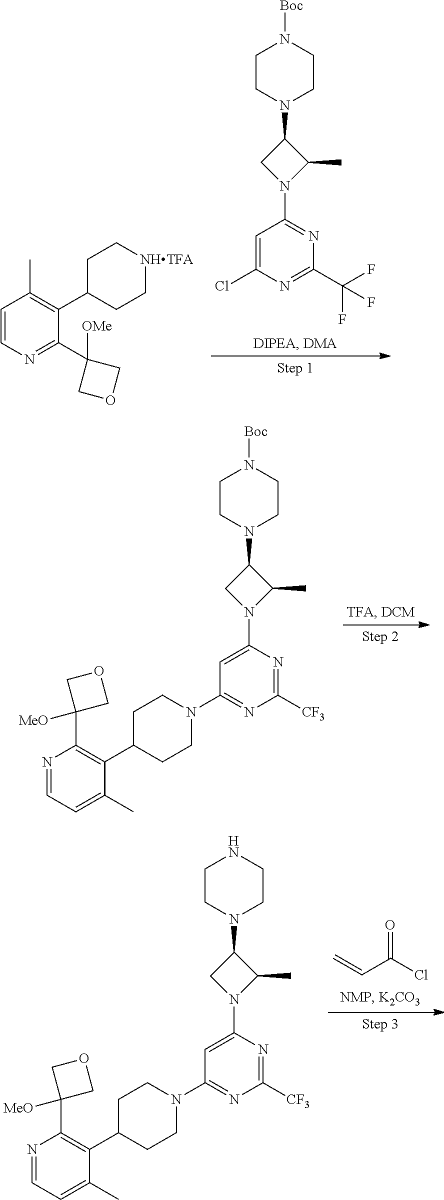

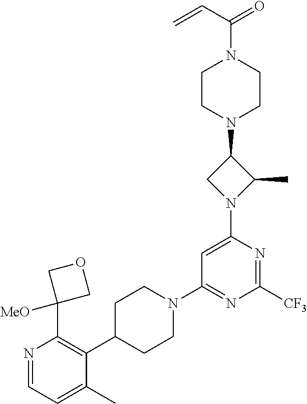 [SMILES: C=CC(=O)N1CCN([C@@H]2CN(c3cc(N4CCC(c5c(C)ccnc5C5(OC)COC5)CC4)nc(C(F)(F)F)n3)[C@@H]2C)CC1]

Step 1. To a stirred solution of 4-(2-(3-methoxyoxetan-3-yl)-4-methylpyridin-3-yl)piperidin-1-yl 2,2,2-trifluoroacetate (85 g, 237 mmol) (Intermediate B-106) in DMA (850 mL) at 0° C., was added DIPEA (207 mL, 1183 mmol) and tert-butyl 4-((2R,3R)-1-(6-chloro-2-(trifluoromethyl)pyrimidin-4-yl)-2-methyl)azetidin-3-yl)piperazine-1-carboxylate (113 g, 260 mmol) and the solution was heated to 110° C. for 16 h. The reaction mixture was quenched with ice cold water (5000 mL) and extracted with EtOAc (2×2500 mL). The combined organic extracts were washed with water (5000 mL), satd. NaCl solution (1000 mL), dried over Na₂SO₄, filtered, and concentrated under reduced pressure. The crude material was purified by chromatography through a Redi-Sep pre-packed silica gel column, eluting with a gradient of 30% to 60% EtOAc in hexanes to give tert-butyl 4-((2R,3R)-1-(6-(4-(2-(3-methoxyoxetan-3-yl)-4-methylpyridin-3-yl)piperidin-1-yl)-2-(trifluoromethyl)pyrimidin-4-yl)-2-methyl)azetidin-3-yl)piperazine-1-carboxylate. m/z (ESI): 662.3 (M+H)⁺. ¹H NMR (400 MHz, DMSO-d₆): δ 8.29 (d, J=4.9 Hz, 1H), 7.18 (d, J=5.2 Hz, 1H), 5.62 (s, 1H), 5.15 (d, J=7.1 Hz, 2H), 4.82 (d, J=7.1 Hz, 2H), 4.38-4.55 (m, 3H), 3.94 (t, J=8.3 Hz, 1H), 3.84 (dd, J=8.9, 6.7 Hz, 1H), 3.35 (s, 4H), 3.23 (q, J=7.2 Hz, 1H), 2.95 (s, 3H), 2.86 (t, J=12.9 Hz, 2H), 2.68-2.80 (m, 1H), 2.37 (s, 3H), 2.24 (s, 4H), 2.01 (dt. J=13.3, 10.2 Hz, 2H t, 1.61 (d, J=12.0 Hz, 2H), 1.39 (d, J=13.1 Hz, 12H).

Step 2. To a stirred solution of tert-butyl 4-((2R,3R)-1-(6-(4-(2-(3-methoxyoxetan-3-yl)-4-methylpyridin-3-yl)piperidin-1-yl)-2-(trifluoromethyl)pyrimidin-4-yl)-2-methyl)azetidin-3-yl)piperazine-1-carboxylate (25 g, 37.8 mmol) in DCM (250 mL) at 0° C., was added TFA (75 mL, 973 mmol) dropwise for 15 min and the reaction mass was stirred at rt for 3 h. The reaction mixture was concentrated under reduced pressure, the crude material was co-evaporated with toluene (3×100 mL), triturated with diethyl ether (2×100 mL), and concentrated under reduced pressure to afford 4-((2R,3R)-1-(6-(4-(2-(3-methoxyoxetan-3-yl)-4-methylpyridin-3-yl)piperidin-1-yl)-2-(trifluoromethyl)pyrimidin-4-yl)-2-methyl)azetidin-3-yl)piperazin-1-yl 2,2,2-trifluoroacetate, which was used directly in the next step without further purification. m/z (ESI): 562.2 (M+H)⁺.

Step 3. To a stirred solution of 4-((2R,3R)-1-(6-(4-(2-(3-methoxyoxetan-3-yl)-4-methylpyridin-3-yl)piperidin-1-yl)-2-(trifluoromethyl)pyrimidin-4-yl)-2-methyl)azetidin-3-yl)piperazin-1-yl 2,2,2-trifluoroacetate (55 g, 83.0 mmol) in NMP (550 mL) at 0° C., was added K₂CO₃ (115.0 g, 834 mmol) slowly for 20 min portion-wise and stirred for 20 min at 0° C. A solution of acryloyl chloride (7.45 mL, 92 mmol) in NMP (5 mL) was added dropwise for 15 min at 0° C., and the reaction mixture was stirred for 15 min at 0° C. The reaction mixture was slowly quenched with ice cold water (400 mL), stirred for 30 min and extracted with EtOAc (2×4000 mL). The combined organic extracts were washed with H₂O (2×4000 mL), followed by satd. NaCl (3000 mL), dried over Na₂SO₄, filtered, and concentrated under vacuum. The crude material was absorbed onto a plug of silica gel and purified by chromatography through a Redi-Sep pre-packed silica gel column (330 g), eluting with a gradient of 2% to 3% MeOH in DCM to provide 1-(4-((2R,3R)-1-(6-(4-(2-(3-methoxyoxetan-3-yl)-4-methylpyridin-3-yl)piperidin-1-yl)-2-(trifluoromethyl)pyrimidin-4-yl)-2-methyl)azetidin-3-yl)piperazin-1-yl)prop-2-en-1-one. m/z (ESI): 616.3 (M+H)⁺. ¹H NMR (400 MHz, DMSO-d₆): δ 8.29 (d, J=4.9 Hz, 1H), 7.18 (d, J=5.0 Hz, 1H), 6.80 (dd, J=16.7, 10.5 Hz, 1H), 6.11 (dd, J=16.7, 2.4 Hz, 1H), 5.60-5.73 (m, 2H), 5.15 (d, J=7.1 Hz, 2H), 4.82 (d, J=7.1 Hz, 2H), 4.49 (dd, J=13.6, 7.0 Hz, 3H), 3.95 (t, J=8.3 Hz, 1H), 3.87 (dd, J=9.0, 6.7 Hz, 1H), 3.49-3.68 (m, 4H), 3.25 (q, J=7.2 Hz, 1H), 2.96 (s, 3H), 2.86 (t, J=13.1 Hz, 2H), 2.74 (td, J=12.2, 6.1 Hz, 1H), 2.37 (s, 3H), 2.29 (s, 4H), 2.01 (tt, J=12.3, 6.9 Hz, 2H), 1.56-1.68 (m, 2H), 1.40 (d, J=6.3 Hz, 3H).

### Example 2-371:1-(4-((2R,3R)-1-(2-(Difluoromethyl)-6-((3S,4R)-3-methyl-4-(4-methyl-1-((S)-tetrahydrofuran-3-yl)-1H-pyrazol-5-yl)piperidin-1-yl)pyrimidin-4-yl)-2-methyl)azetidin-3-yl)piperazin-1-yl)prop-2-en-1-one

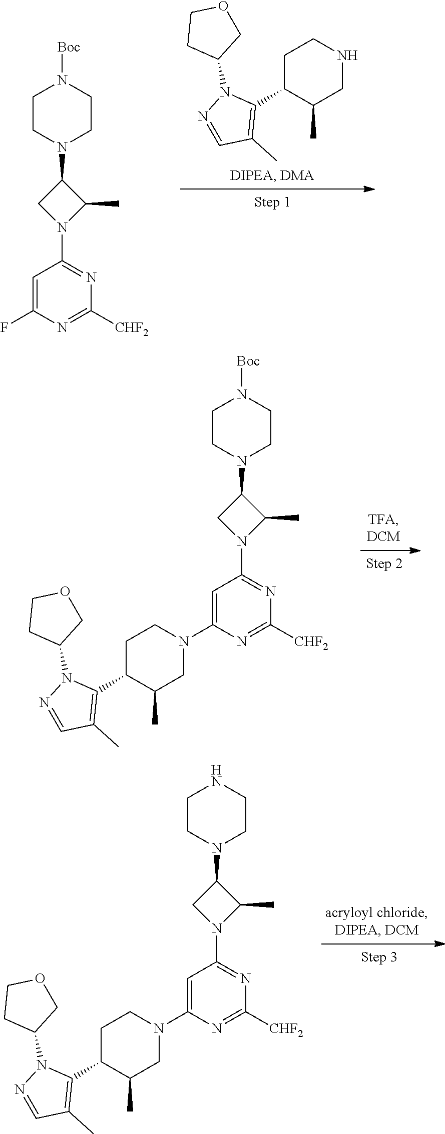 [SMILES: C[C@@H]1[C@H](N2CCN(C(=O)OC(C)(C)C)CC2)CN1c1cc(F)nc(C(F)F)n1 ; Cc1cnn([C@@H]2CCOC2)c1[C@@H]1CCNC[C@H]1C ; Cc1cnn([C@@H]2CCOC2)c1[C@@H]1CCN(c2cc(N3C[C@@H](N4CCN(C(=O)OC(C)(C)C)CC4)[C@H]3C)nc(C(F)F)n2)C[C@H]1C ; Cc1cnn([C@@H]2CCOC2)c1[C@@H]1CCN(c2cc(N3C[C@@H](N4CCNCC4)[C@H]3C)nc(C(F)F)n2)C[C@H]1C]

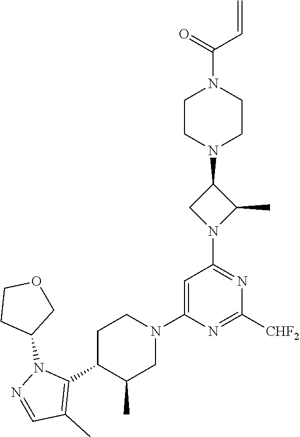 [SMILES: C=CC(=O)N1CCN([C@@H]2CN(c3cc(N4CC[C@@H](c5c(C)cnn5[C@@H]5CCOC5)[C@H](C)C4)nc(C(F)F)n3)[C@@H]2C)CC1]

Step 1. To a stirred solution of (3S,4R)-3-methyl-4-(4-methyl-1-((S)-tetrahydrofuran-3-yl)-1H-pyrazol-5-yl)piperidine (3.4 g, 13.64 mmol) in DMA (60 mL) was added DIPEA (11.91 mL, 68.2 mmol) at 0° C. Then, tert-butyl 4-((2R,3R)-1-(2-(difluoromethyl)-6-fluoropyrimidin-4-yl)-2-methyl)azetidin-3-yl)piperazine-1-carboxylate (6.02 g, 15.00 mmol) was added at RT. The resulting reaction mixture was stirred at 110° C. for 16 h. The reaction mixture was diluted with ice cold water (100 mL) and extracted with EtOAc (2×50 mL). The combined organic extracts were washed with brine (2×50 mL) and dried over Na₂SO₄. The solution was filtered and concentrated in vacuo to give the crude material, which was purified by chromatography on silica gel, eluting with a gradient of 0% to 50% EtOAc in hexanes, to provide tert-butyl 4-((2R,3R)-1-(2-(difluoromethyl)-6-((3S,4R)-3-methyl-4-(4-methyl-1-((S)-tetrahydrofuran-3-yl)-1H-pyrazol-5-yl)piperidin-1-yl)pyrimidin-4-yl)-2-methyl)azetidin-3-yl)piperazine-1-carboxylate. m/z (ESI): 631.3 (M+H)⁺.

Step 2. To a vessel was added tert-butyl 4-((2R,3R)-1-(2-(difluoromethyl)-6-((3S,4R)-3-methyl-4-(-(4-methyl-1-((S)-tetrahydrofuran-3-yl)-1H-pyrazol-5-yl)piperidin-1-yl)pyrimidin-4-yl)-2-methyl)azetidin-3-yl)piperazine-1-carboxylate (7 g, 11.10 mmol) and DCM (70.0 mL). The reaction mixture was cooled to 0° C. and TFA (17.10 mL, 222 mmol) was added. The reaction mixture was stirred at 0° C. for 2 h. The reaction mixture was concentrated in vacuo and co-distilled with toluene (2×75 mL), washed with diethyl ether (2×75 mL) and concentrated in vacuo to give the crude material that was taken to next step. m/z (ESI): 531.3 (M+H)⁺.

Step 3. To a solution of 1-(4-((2R,3R)-1-(2-(difluoromethyl)-6-((3S,4R)-3-methyl-4-(4-methyl-1-((S)-tetrahydrofuran-3-yl)-1H-pyrazol-5-yl)piperidin-1-yl)pyrimidin-4-yl-2-methyl)azetidin-3-yl)-114-piperazin-1-yl)-2,2,2-trifluoroethan-1-one (7 g, 1.15 mmol) in NMP (140 mL) at 0° C. was added K₂CO₃ (15.41 g, 112 mmol). The reaction mixture was stirred at 0° C. for 15 min. Acryloyl chloride (0.997 mL, 12.27 mmol) (in 5 mL NMP) was added to the reaction mixture and the mixture was stirred for 30 min. The reaction mixture was diluted with ice cold water (1000 mL) and extracted with EtOAc (3×300 mL). The organic extracts were washed with ice cold water (3×500 mL) followed by brine (500 mL) and dried over Na₂SO₄. The solution was filtered and, concentrated in vacuo to give the crude material which was purified by chromatography through a Redi-Sep prepacked silica gel column (120 g), eluting with a gradient of 0-5% MeOH in DCM, to provide 1-(4-((2R,3R)-1-(2-(difluoromethyl)-6-((3S,4R)-3-methyl-4-(4-methyl-1-((S)-tetrahydrofuran-3-yl)-1H-pyrazol-5-yl)piperidin-1-yl)pyrimidin-4-yl)-2-methyl)azetidin-3-yl)piperazin-1-ylprop-2-en-1-one. m/z (ESI): 585.2 (M+H)⁺. ¹H NMR (400 MHz, DMSO-d₆) δ 7.21 (s, 1H), 6.81 (dd, J=16.7, 10.5 Hz, 1H), 6.43 (t, J=54.9 Hz, 1H), 6.11 (dd, J=16.7, 2.4 Hz, 1H), 5.69 (dd, J=10.5, 2.4 Hz, 1H), 5.58 (s, 1H), 5.12 (s, 1H), 4.48 (p, J=6.4 Hz, 3H), 3.89-4.09 (m, 3H), 3.85 (dd, J=8.4, 6.5 Hz, 2H), 3.72 (dd, J=8.6, 5.1 Hz, 1H), 3.24 (q, J=7.2 Hz, 1H), 2.89 (dt, J=13.5, 6.0 Hz, 2H), 2.55-2.67 (m, 1H), 2.27 (d, J=18.4 Hz, 6H), 1.95 (s, 4H), 1.73 (s, 2H), 1.40 (d, J=6.3 Hz, 3H), 0.70 (d, J=6.5 Hz, 3H).

### Example 2-372:1-(4-((2R,3R)-1-(6-((2R,4S)-4-(2-((S)-3-Methoxytetrahydrofuran-3-yl)-4-methylpyridin-3-yl)-2-methylpiperidin-1-yl)-2-(trifluoromethyl)pyrimidin-4-yl)-2-methyl)azetidin-3-yl)piperazin-1-yl)prop-2-en-1-one

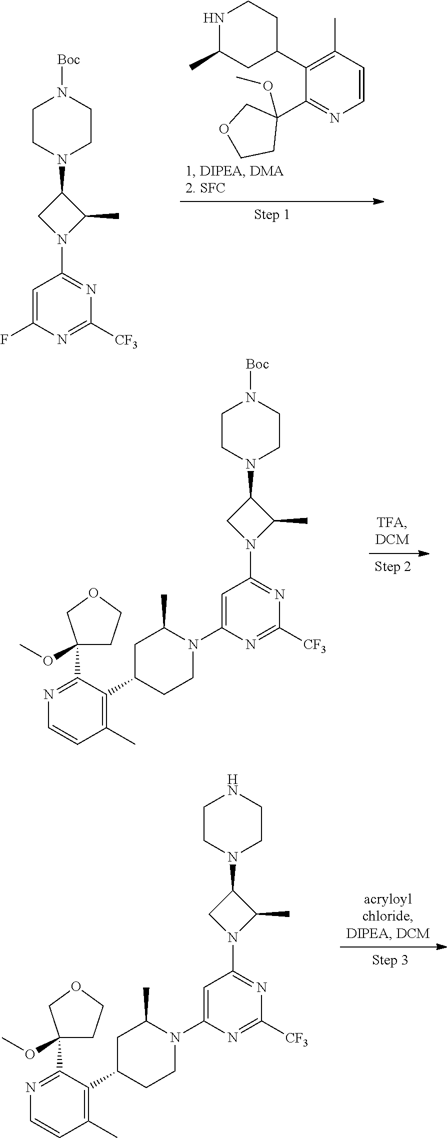 [SMILES: C[C@@H]1[C@H](N2CCN(C(=O)OC(C)(C)C)CC2)CN1c1cc(F)nc(C(F)(F)F)n1 ; COC1(c2nccc(C)c2C2CCN[C@H](C)C2)CCOC1 ; CO[C@]1(c2nccc(C)c2[C@H]2CCN(c3cc(N4C[C@@H](N5CCN(C(=O)OC(C)(C)C)CC5)[C@H]4C)nc(C(F)(F)F)n3)[C@H](C)C2)CCOC1 ; CO[C@]1(c2nccc(C)c2[C@H]2CCN(c3cc(N4C[C@@H](N5CCNCC5)[C@H]4C)nc(C(F)(F)F)n3)[C@H](C)C2)CCOC1]

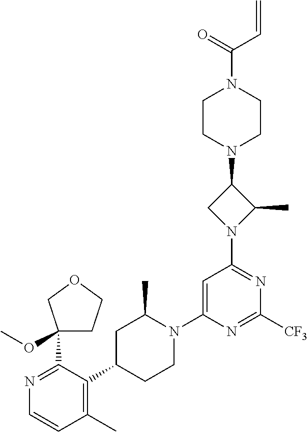 [SMILES: C=CC(=O)N1CCN([C@@H]2CN(c3cc(N4CC[C@H](c5c(C)ccnc5[C@@]5(OC)CCOC5)C[C@H]4C)nc(C(F)(F)F)n3)[C@@H]2C)CC1]

Step 1. To a vessel was added 2-(3-methoxytetrahydrofuran-3-yl)-4-methyl-3-((2R)-2-methylpiperidin-4-yl)pyridine hydrochloride (0.748 g, 2.289 mmol) and tert-butyl 4-((2R,3R)-1-(6-fluoro-2-(trifluoromethyl)pyrimidin-4-yl)-2-methyl)azetidin-3-yl)piperazine-1-carboxylate (0.8 g, 1.907 mmol) and DIPEA (1.666 mL, 9.54 mmol) in DMA (16.00 mL). The reaction mixture was stirred at 110° C. for 16 h. The reaction mixture was diluted with H₂O and extracted with EtOAc. The organic extract was dried over Na₂SO₄. The solution was filtered and concentrated in vacuo to give the crude material that was purified by chromatography through a Redi-Sep prepacked silica gel column (12 g), eluting with a gradient of 30-35% EtOAc in hexanes, to provide tert-butyl 4-((2R,3R)-1-(6-((2R)-4-(2-(3-methoxytetrahydrofuran-3-yl)-4-methylpyridin-3-yl)-2-methylpiperidin-1-yl)-2-(trifluoromethyl)pyrimidin-4-yl)-2-methyl)azetidin-3-yl)piperazine-1-carboxylate which was purified by SFC with a Chiralpak IC column. using 50% (1:1) ACN:MeOH to provide:
Peak 1. tert-Butyl 4-((2R,3R)-1-(2-(difluoromethyl)-6-(2R,4S)-4-(2-((S)-3-methoxytetrahydrofuran-3-yl)-4-methylpyridin-3-yl)-2-methylpiperidin-1-yl)pyrimidin-4-yl)-2-methyl)azetidin-3-yl)piperazine-1-carboxylate. m/z (ESI): 690.3 (M+H)⁺. ¹H NMR (400 MHz, DMSO-d₆): δ (ppm) 8.24 (d, J=4.8 Hz, 1H), 7.13 (d, J=4.9 Hz, 1H), 5.57 (s, 1H), 4.49 (q, J=6.5 Hz, 1H), 4.23 (d, J=9.7 Hz, 1H), 4.08 (d, J=9.7 Hz, 1H), 3.87 (m, 4H), 3.32 (s, 8H), 3.23 (q, J=7.2 Hz, 1H), 2.96 (s, 3H), 2.76 (dt, J=13.1, 8.1 Hz, 11H), 2.45 (s, 3H), 2.37 (m, 2H), 2.24 (s, 4H), 2.08 (t, J=6.0 Hz, 1H), 1.60 (dd, J=33.1, 12.7 Hz, 2H), 1.39 (d, J=12.0 Hz, 12H), 1.23 (d, J=6.9 Hz, 3H).
Peak 2. tert-Butyl 4-((2R,3R)-1-(2-(difluoromethyl)-6-((2R,4S)-4-(2-((R)-3-methoxytetrahydrofuran-3-yl)-4-methylpyridin-3-yl)-2-methylpiperidin-1-yl)pyrimidin-4-yl)-2-methyl)azetidin-3-yl)piperazine-1-carboxylate. m/z (ESI): 690.3 (M+H)⁺. ¹H NMR (400 MHz, DMSO-d₆): δ (ppm) 8.22 (d, J=4.9 Hz, 1H), 7.13 (d, J=4.9 Hz, 1H), 5.58 (s, 1H), 4.49 (q, J=6.5 Hz, 1H), 4.20 (m, 2H), 4.07 (t, J=12.9 Hz, 1H), 3.95 (q, J=7.6 Hz, 2H), 3.81 (m, 2H), 3.32 (s, 8H), 3.23 (q, J=7.2 Hz, 1H), 2.96 (s, 3H), 2.58 (dt, J=13.0, 7.8 Hz, 1H), 2.45 (s, 4H), 2.24 (s, 4H), 2.09 (d, J=9.6 Hz, 1H), 1.70 (d, J=12.8 Hz, 1H), 1.52 (d, J=12.8 Hz, 1H), 1.39 (d, J=12.1 Hz, 12H), 1.23 (d, J=6.8 Hz, 3H).

Step 2. To a vessel H as added tert-butyl 4-((2R,3R)-1-(2-(difluoromethyl)-6-((2R,4S)-4-(2-((S)-3-methoxytetrahydrofuran-3-yl)-4-methylpyridin-3-yl)-2-methylpiperidin-1-yl)pyrimidin-4-yl)-2-methyl)azetidin-3-yl)piperazine-1-carboxylate (0.22 g, 0.319 mmol) (peak 1) and TFA (0.246 mL, 3.19 mmol) in DCM (4.40 mL). The reaction mixture was stirred at rt for 1 h. The solution was concentrated in vacuo to give 2-(difluoromethyl)-4-((2R,4S)-4-(2-((S)-3-methoxytetrahydrofuran-3-yl)-4-methylpyridin-3-yl)-2-methylpiperidin-1-yl)-6-((2R,3R)-2-methyl-3-(piperazin-1-yl)azetidin-1-yl)pyrimidine 2,2,2-trifluoroacetate, which was used as is for the next step. m/z (ESI): 572.3 (M+H)⁺.

Step 3. To a vessel was added 4-((2R,4S)-4-(2-((S)-3-methoxy tetrahydrofuran-3-yl)-4-methylpyridin-3-yl)-2-methylpiperidin-1-yl)-6-((2R,3R)-2-methyl-3-(piperazin-1-yl)azetidin-1-yl)-2-(trifluoromethyl)pyrimidine 2,2,2-trifluoroacetate (0.27 g, 0.384 mmol). DIPEA (0.335 mL, 1.918 mmol) and DCM (5.40 mL). To the reaction mixture was added acryloyl chloride (0.034 mL, 0.422 mmol) at −78° C., and the reaction mixture was stirred for 15 min. The reaction mixture was diluted with H₂O and extracted with DCM. The organic extract was dried over Na₂SO₄. The solution was filtered and concentrated in vacuo to give the crude material that was purified by prep HPLC to provide 1-(4-((2R,3R)-1-(6-((2R,4S)-4-(2-((S)-3-methoxytetrahydrofuran-3-yl)-4-methylpyridin-3-yl)-2-methylpiperidin-1-yl)-2-(trifluoromethyl)pyrimidin-4-yl)-2-methyl)azetidin-3-yl)piperazin-1-yl)prop-2-en-1-one. m/z (ESI): 644.3 (M+H)⁺. ¹H NMR (400 MHz, DMSO-d₆): δ (ppm) 8.24 (d, J=4.9 Hz, 1H), 7.13 (d, J=4.9 Hz, 11H), 6.81 (dd, J=16.7, 10.5 Hz, 1H), 6.42 (t, J=54.9 Hz, 1H), 6.11 (dd, J=16.6, 2.4 Hz, 1H), 5.69 (dd, J=10.4, 2.4 Hz, 1H), 5.51 (s, 1H), 4.48 (p, J=6.4 Hz, 1H), 4.23 (d, J=9.7 Hz, 1H), 4.08 (d, J=9.6 Hz, 2H), 3.86 (m, 5H), 3.58 (s, 4H), 3.24 (q, J=7.2 Hz, 1H), 2.97 (s, 4H), 2.75 (dt, J=13.1, 8.0 Hz, 2H), 2.45 (s, 3H), 2.35 (m, 6H), 2.06 (m, 1H), 1.59 (dd, J=32.1, 12.9 Hz, 2H), 1.40 (d, J=6.3 Hz, 3H), 1.22 (d, J=6.8 Hz, 3H).

### Example 2-373: 1-(4-((2R,3R)-1-(6-((2R,4S)-4-(2-((R)-3-Methoxytetrahydrofuran-3-yl)-4-methylpyridin-3-yl)-2-methylpiperidin-1-yl)-2-(trifluoromethyl)pyrimidin-4-yl)-2-methyl)azetidin-3-yl)piperazin-1-yl)prop-2-en-1-one

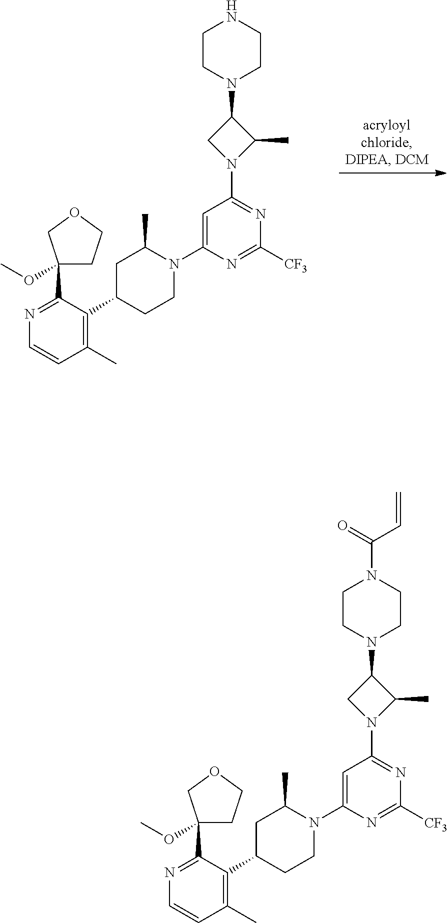 [SMILES: CO[C@@]1(c2nccc(C)c2[C@H]2CCN(c3cc(N4C[C@@H](N5CCNCC5)[C@H]4C)nc(C(F)(F)F)n3)[C@H](C)C2)CCOC1 ; C=CC(=O)N1CCN([C@@H]2CN(c3cc(N4CC[C@H](c5c(C)ccnc5[C@]5(OC)CCOC5)C[C@H]4C)nc(C(F)(F)F)n3)[C@@H]2C)CC1]

4-((2R,4S)-4-(2-((R)-3-methoxytetrahydrofuran-3-yl)-4-methylpyridin-3-yl)-2-methylpiperidin-1-yl)-6-((2R,3R)-2-methyl-3-(piperazin-1-yl)azetidin-1-yl)-2-(trifluoromethyl)pyrimidine TFA (0.27 g, 0.384 mmol), prepared as described for Example 2-372, steps 1 and 2. was mixed with DIPEA (0.335 mL, 1.918 mmol) in DCM (5.40 mL). To the reaction mixture was added acryloyl chloride (0.034 mL, 0.422 mmol) at −78° C., and the reaction mixture was stirred for 15 mins. The reaction mixture was diluted with H₂O and extracted with DCM. The organic extract was dried over Na₂SO₄. The solution was filtered and concentrated in vacuo to give the crude material. Purification by prep. HPLC provided 1-(4-((2R,3R)-1-(6-((2R,4S)-4-(2-((SR)-3-methoxytetrahydrofuran-3-yl)-4-methylpyridin-3-yl)-2-methylpiperidin-1-yl)-2-(trifluoromethyl)pyrimidin-4-yl)-2-methyl)azetidin-3-yl)piperazin-1-yl)prop-2-en-1-one. m/z (ESI): 644.3 (M+H)⁺. ¹H NMR (400 MHz, DMSO-d₆) δ 8.22 (d, J=4.8 Hz, 1H), 7.13 (d, J=4.9 Hz, 1H), 6.81 (dd, J=16.7, 10.5 Hz, 1H), 6.11 (dd, J=16.6, 2.4 Hz, 2H), 5.75-5.43 (m, 2H), 4.49 (q, J=6.5 Hz, 1H), 4.27-4.15 (m, 2H), 4.13-3.99 (m, 1H), 4.00-3.91 (m, 2H), 3.88 (dd, J=8.9, 6.7 Hz, 1H), 3.78 (td, J=8.2, 5.1 Hz, 1H), 3.59 (s, 4H), 3.25 (q, J=7.2 Hz, 2H), 2.96 (s, 3H), 2.63-2.53 (m, 1H), 2.45 (s, 4H), 2.29 (s, 5H), 2.10 (s, 2H), 1.70 (d, J=12.8 Hz, 1H), 1.52 (d, J=13.1 Hz, 1H), 1.40 (d, J=6.3 Hz, 3H), 1.23 (d, J=6.7 Hz, 3H).

### Example 2-374: 1-(4-((2R,3R)-1-(2-(Difluoromethyl)-6-((2R,4S)-4-(2-((R)-3-methoxytetrahydrofuran-3-yl)-4-methylpyridin-3-yl)-2-methylpiperidin-1-yl)pyrimidin-4-yl)-2-methyl)azetidin-3-yl)piperazin-1-yl)prop-2-en-1-one

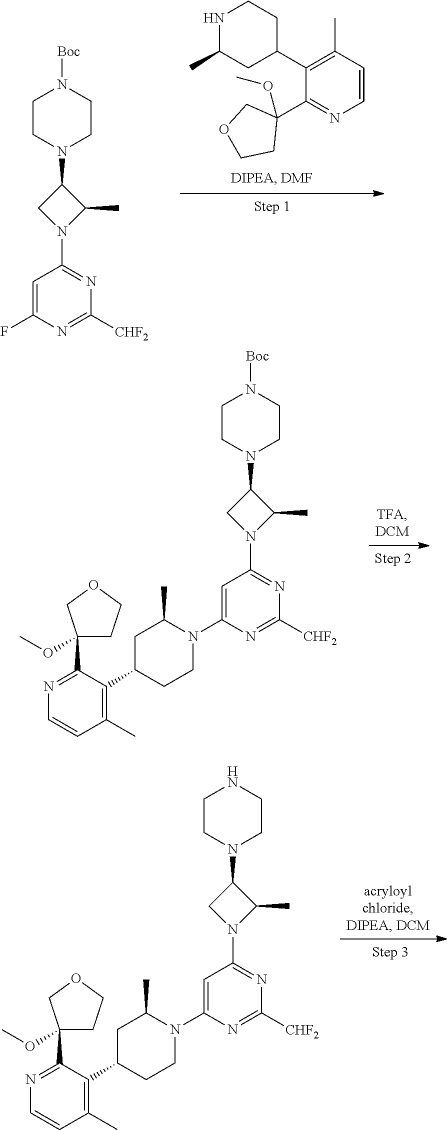 [SMILES: C[C@@H]1[C@H](N2CCN(C(=O)OC(C)(C)C)CC2)CN1c1cc(F)nc(C(F)F)n1 ; COC1(c2nccc(C)c2C2CCN[C@H](C)C2)CCOC1 ; CO[C@@]1(c2nccc(C)c2[C@H]2CCN(c3cc(N4C[C@@H](N5CCN(C(=O)OC(C)(C)C)CC5)[C@H]4C)nc(C(F)F)n3)[C@H](C)C2)CCOC1 ; CO[C@@]1(c2nccc(C)c2[C@H]2CCN(c3cc(N4C[C@@H](N5CCNCC5)[C@H]4C)nc(C(F)F)n3)[C@H](C)C2)CCOC1]

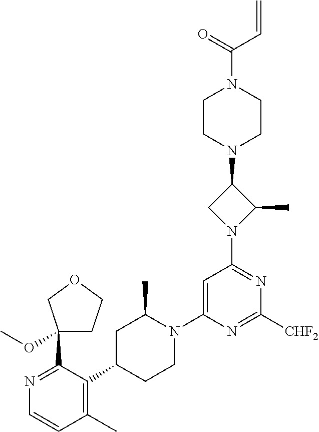 [SMILES: C=CC(=O)N1CCN([C@@H]2CN(c3cc(N4CC[C@H](c5c(C)ccnc5[C@]5(OC)CCOC5)C[C@H]4C)nc(C(F)F)n3)[C@@H]2C)CC1]

Step 1. To a vessel was added 2-(3-methoxytetrahydrofuran-3-yl)-4-methyl-3-((2R)-2-methylpiperidin-4-yl)pyridine hydrochloride (0.65 g, 1.989 mmol) and tert-butyl 4-((2R,3R)-1-(2-(difluoromethyl)-6-fluoropyrimidin-4-yl)-2-methyl)azetidin-3-yl)piperazine-1-carboxylate (0.665 g, 1.657 mmol) and DIPEA (1.447 mL, 8.29 mmol) in DMA (13.00 mL). The reaction mixture was stirred at 110° C. for 16 h. The reaction mixture was diluted with H₂O and extracted with EtOAc. The organic extract was dried over Na₂SO₄. The solution was filtered and concentrated in vacuo to give the crude material, which was purified by chromatography through a Redi-Sep prepacked silica gel column (12 g), eluting with a gradient of 70-75% EtOAc in hexanes, to provide tert-butyl 4-((2R,3R)-1-(2-(difluoromethyl)-6-((2R)-4-(2-(3-methoxytetrahydrofuran-3-yl)-4-methylpyridin-3-yl)-2-methylpiperidin-1-yl)pyrimidin-4-yl)-2-methyl)azetidin-3-yl)piperazine-1-carboxylate, which was further purified by SFC using a Chiralpak IC, 150×50 mm 5μ, column with a mobile phase of 50% (1:1 ACN:MeOH in liquid CO₂ using a flowrate of 180 mL/mm to give:
Peak 1. tert-butyl 4-((2R,3R)-1-(2-(difluoromethyl)-6-((2R,4S)-4-(2-((R)-3-methoxytetrahydrofuran-3-yl)-4-methylpyridin-3-yl)-2-methylpiperidin-1-yl)pyrimidin-4-yl)-2-methyl)azetidin-3-yl)piperazine-1-carboxylate. ¹H NMR (400 MHz, DMSO-d₆): δ (ppm) 8.24 (d, J=4.9 Hz, 1H), 7.13 (d, J=5.0 Hz, 1H), 6.42 (t, J=54.9 Hz, 1H), 5.50 (s, 1H), 4.46 (p, J=6.4 Hz, 1H), 4.23 (d, J=9.7 Hz, 1H), 4.10 (q, J=5.3 Hz, 5H), 3.99 (t, J=12.9 Hz, 1H), 3.85 (m, 4H), 3.17 (d, J=5.3 Hz, 1H), 2.96 (s, 3H), 2.75 (dt, J=13.2, 8.0 Hz, 1H), 2.24 (s, 4H), 1.59 (dd, J=31.8, 12.6 Hz, 2H), 1.39 (d, J=13.5 Hz, 12H), 1.22 (d, J=6.9 Hz, 3H)
Peak 2. tert-butyl 4-((2R,3R)-1-(2-(difluoromethyl)-6-((2R,4S)-4-(2-((S)-3-methoxytetrahydrofuran-3-yl)-4-methylpyridin-3-yl)-2-methylpiperidin-1-yl)pyrimidin-4-yl)-2-methyl)azetidin-3-yl)piperazine-1-carboxylate. ¹H NMR (400 MHz, DMSO-d₆), δ (ppm) 8.22 (d, J=4.9 Hz, 1H), 7.13 (d, J=5.0 Hz, 1H), 6.42 (t, J=54.9 Hz, 1H), 5.51 (s, 1H), 4.46 (t, J=6.6 Hz, 1H), 4.21 (d, J=4.1 Hz, 2H), 4.10 (q, J=5.3 Hz, 7H), 3.94 (p, J=8.8, 8.2 Hz, 2H), 3.80 (m, 2H), 3.35 (s, 2H), 3.35 (s, 4H), 3.23 (m, 1H), 2.96 (s, 3H), 2.58 (m, 1H), 2.44 (s, 4H), 2.30 (m, 4H), 1.69 (d, J=12.8 Hz, 1H), 1.40 (d, J=7.3 Hz, 13H), 1.22 (d, J=6.9 Hz, 3H).

Step 2: To a vessel was added (peak 21) tert-butyl 4-((2R,3R)-1-(2-(difluoromethyl)-6-((2R,4S)-4-(2-((SR)-3-methoxy)tetrahydrofuran-3-yl)-4-methylpyridin-3-yl)-2-methylpiperidin-1-yl)pyrimidin-4-yl)-2-methyl)azetidin-3-yl)piperazine-1-carboxylate (0.25 g, 0.372 mmol) and TFA (0.287 mL, 3.72 mmol) in DCM (2.5 mL). The reaction mixture was stirred at RT for 1 h, then concentrated under reduced pressure. The reaction mixture was diluted with NaHCO₃ solution and extracted with DCM. The organic extract was dried over Na₂SO₄. The solution was filtered and concentrated in vacuo to give the crude material, which was purified by prep. HPLC to provide 2-(difluoromethyl)-4-((2R,4S)-4-(2-(-((R)-3-methoxy tetrahydrofuran-3-yl)-4-methylpyridin-3-yl)-2-methylpiperidin-1-yl)-6-((2R,3R)-2-methyl-3-(piperazin-1-yl)azetidin-1-yl)pyrimidine. ¹H NMR (400 MHz, DMSO-d₆): δ (ppm) 8.22 (d, J=4.9 Hz, 1H), 7.13 (d, J=4.9 Hz, 1H), 6.42 (t, J=54.9 Hz, 1H), 5.49 (s, 1H), 4.44 (q, J=6.5 Hz, 1H), 4.20 (m, 2H), 4.07 (t, J=12.9 Hz, 1H), 3.92 (m, 2H), 3.79 (ddd, J=11.0, 9.1, 5.9 Hz, 2H), 3.17 (t, J=7.2 Hz, 1H), 2.96 (s, 3H), 2.71 (m, 4H), 2.52 (s, 6H), 2.45 (s, 3H), 2.20 (s, 4H), 2.08 (m, 11H), 1.69 (d, J=12.7 Hz, 11H), 1.51 (d, J=12.9 Hz, 1H), 1.36 (d, J=6.3 Hz, 3H), 1.22 (d, J=6.9 Hz, 3H).

Step 3: To a vessel was added 2-(difluoromethyl)-4-((2R,4S)-4-(2-(R)-3-methoxytetrahydrofuran-3-yl)-4-methylpyridin-3-yl)-2-methylpiperidin-1-yl)-6-((2R,3R)-2-methyl-3-(piperazin-1-yl)azetidin-1-ylpyrimidine (0.12 g, 0.210 mmol). DIPEA (0.183 mL, 1.049 mmol), and DCM (5 mL). To the reaction mixture was added acryloyl chloride (0.019 mL, 0.231 mmol) at −78° C. and the reaction mixture was stirred at −78° C. for 30 min. The reaction mixture was diluted with H₂O and extracted with DCM. The organic extract was dried over Na₂SO₄. The solution was filtered and concentrated in vacuo to give the crude material, which was purified by prep. HPLC to provide 1-(4-((2R,3R)-1-(2-(difluoromethyl)-6-((2R,4S)-4-(2-((R)-3-methoxytetrahydrofuran-3-yl)-4-methylpyridin-3-yl)-2-methylpiperidin-1-yl)pyrimidin-4-yl)-2-methyl)azetidin-3-yl)piperazin-1-yl)prop-2-en-1-one. m/z (ESI): 626.4 (M+H)⁺. ¹H NMR (400 MHz DMSO-d₆): δ (ppm) 8.22 (d, J=4.8 Hz, 1H), 7.13 (d, J=5.0 Hz, 11H), 6.81 (dd, J=16.7, 10.5 Hz, 11H), 6.42 (t, J=54.9 Hz, 1H), 6.11 (dd, J=16.7, 2.4 Hz, 1H), 5.69 (dd, J=10.4, 2.4 Hz, 2H), 5.51 (s, 1H), 4.48 (p, J=6.4 Hz, 11H), 4.20 (m, 2H), 4.07 (t, J=13.0 Hz, 1H), 3.87 (m, 4H), 3.59 (s, 3H), 3.24 (q, J=7.1 Hz, 1H), 3.01 (s, 1H), 2.62 (m, 1H), 2.45 (s, 6H), 2.32 (m, 6H), 2.09 (dt, J=15.2, 10.4 Hz, 1H), 1.69 (d, J=12.6 Hz, 1H), 1.51 (d, J=12.9 Hz, 1H), 1.40 (d, J=6.3 Hz, 3H), 1.23 (d, J=6.8 Hz, 3H).

### Example 2-375: 1-(4-((2R,3R)-1-(2-(difluoromethyl)-6-((2R,4S)-4-(2-((S)-3-methoxytetrahydrofuran-3-yl)-4-methylpyridin-3-yl)-2-methylpiperidin-1-yl)pyrimidin-4-yl)-2-methyl)azetidin-3-yl)piperazin-1-yl)prop-2-en-1-one

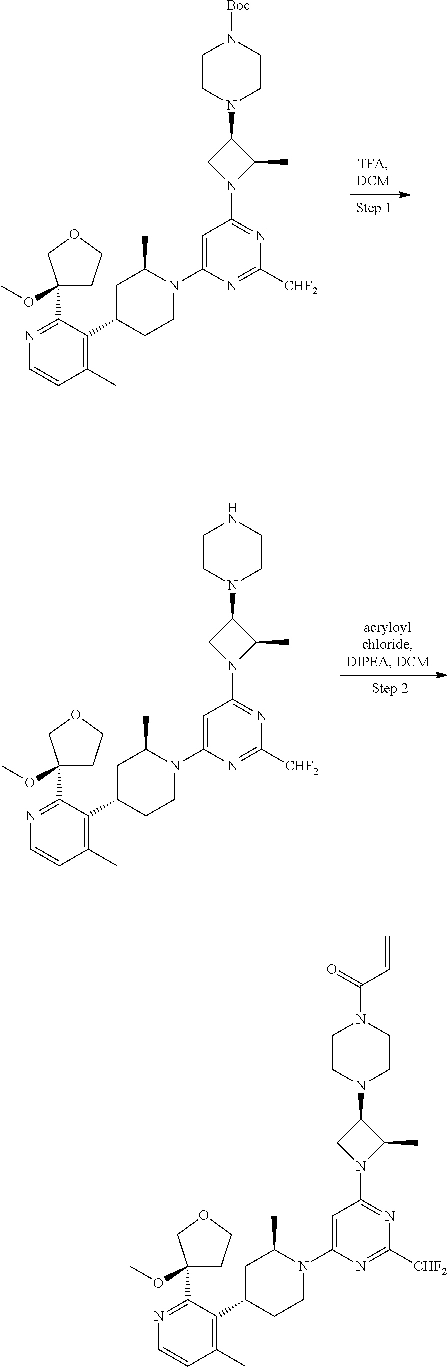 [SMILES: CO[C@]1(c2nccc(C)c2[C@H]2CCN(c3cc(N4C[C@@H](N5CCN(C(=O)OC(C)(C)C)CC5)[C@H]4C)nc(C(F)F)n3)[C@H](C)C2)CCOC1 ; CO[C@]1(c2nccc(C)c2[C@H]2CCN(c3cc(N4C[C@@H](N5CCNCC5)[C@H]4C)nc(C(F)F)n3)[C@H](C)C2)CCOC1 ; C=CC(=O)N1CCN([C@@H]2CN(c3cc(N4CC[C@H](c5c(C)ccnc5[C@@]5(OC)CCOC5)C[C@H]4C)nc(C(F)F)n3)[C@@H]2C)CC1]

Step 1. To a vessel was added tert-butyl 4-((2R,3R)-1-(2-(difluoromethyl)-6-((2R,4S)-4-(2-((S)-3-methoxytetrahydrofuran-3-yl)-4-methylpyridin-3-yl)-2-methylpiperidin-1-yl)pyrimidin-4-yl)-2-methyl)azetidin-3-yl)piperazine-1-carboxylate (0.26 g, 0.387 mmol) and TFA (0.298 mL, 3.87 mmol) in DCM (2.6 mL). The reaction mixture was stirred at RT for 1 h, then concentrated under reduced pressure. The reaction mixture was diluted with NaHCO₃ solution and extracted with DCM. The organic extract was dried over Na₂SO₄. The solution was filtered and concentrated in vacuo to give the crude material, which was purified by prep. HPLC, to provide 2-(difluoromethyl)-4-((2R,4S)-4-(2-((S)-3-methoxytetrahydrofuran-3-yl)-4-methylpyridin-3-yl)-2-methylpiperidin-1-yl)-6-((2R,3R)-2-methyl-3-(piperazin-1-yl)azetidin-1-yl)pyrimidine. ¹H NMR (400 MHz, DMSO-d₆): δ (ppm) 8.24 (d, J=4.8 Hz, 1H), 7.13 (d, J=5.0 Hz, 1H), 6.42 (t, J=54.9 Hz, 1H), 5.49 (s, 1H), 4.45 (p, J=−6.3 Hz, 1H), 4.23 (d, J=9.7 Hz, 1H), 4.08 (d, J=9.7 Hz, 1H), 3.97 (m, 1H), 3.89 (m, 2H), 3.80 (td, J=7.8, 4.0 Hz, 2H), 3.16 (q, J=7.2 Hz, 1H), 2.96 (s, 3H), 2.73 (m, 6H), 2.45 (s, 3H), 2.36 (m, 2H), 2.13 (d, J=55.3 Hz, 7H), 1.59 (dd, J=31.6, 12.8 Hz, 2H), 1.36 (d, J=6.3 Hz, 3H), 1.22 (d, J=6.8 Hz, 3H).

Step 2: To a vessel was added 2-(difluoromethyl)-4-((2R,4S)-4-(2-((S)-3-methoxytetrahydrofuran-3-yl)-4-methylpyridin-3-yl)-2-methylpiperidin-1-yl)-6-((2R,3R)-2-methyl-3-(piperazin-1-yl)azetidin-1-yl)pyrimidine (0.13 g, 0.227 mmol), DIPEA (0.199 mL, 1.137 mmol), and DCM (1.300 mL). To the reaction mixture was added acryloyl chloride (0.020 mL, 0.250 mmol) at −78° C. The reaction mixture was stirred at −78° C. for 30 mins. The reaction mixture was diluted with H₂O and extracted with DCM. The organic extract was dried over Na₂SO₄. The solution was filtered and, concentrated in vacuo to give the crude material, which was purified by prep. HPLC to provide 1-(4-((2R,3R)-1(2-(difluoromethyl)-6-((2R,4S)-4-(2-((S)-3-methoxytetrahydrofuran-3-yl)-4-methylpyridin-3-yl)-2-methylpiperidin-1-yl)pyrimidin-4-yl)-2-methyl)azetidin-3-yl)piperazin-1-yl)prop-2-en-1-one. m/z (ESI): 626.4 (M+H)⁺. ¹H NMR (400 MHz, DMSO-d₆): δ (ppm) 8.24 (d, J=4.9 Hz, 1H), 7.13 (d, J=5.0 Hz, 1H), 6.81 (dd, J=16.7, 10.4 Hz, 1H), 6.43 (t, J=54.9 Hz, 1H), 6.11 (dd, J=16.7, 2.4 Hz, 1H), 5.69 (dd, J=10.4, 2.4 Hz, 1H), 5.51 (s, 1H), 4.48 (p, J=6.4 Hz, 1H), 4.23 (d, J=9.7 Hz, 1H), 4.08 (d, J=9.7 Hz, 1H), 3.87 (m, 4H), 3.59 (s, 4H), 3.24 (q, J=7.1 Hz, 1H), 2.97 (s, 3H), 2.73 (m, 2H), 2.45 (s, 3H), 2.37 (m, 2H), 2.29 (s, 6H), 2.08 (td, J=13.9, 9.6 Hz, 1H), 1.59 (dd, J=31.6, 12.7 Hz, 2H), 1.40 (d, J=6.3 Hz, 3H), 1.22 (d, J=6.8 Hz, 3H).

### Example 2-376: 1-(4-((2R,3R)-1-(6-((2R,4S)-4-(2-(3-methoxyoxetan-3-yl)-4-methylpyridin-3-yl)-2-methylpiperidin-1-yl)-2-(trifluoromethyl)pyrimidin-4-yl)-2-methyl)azetidin-3-yl)piperazin-1-yl)prop-2-en-1-one

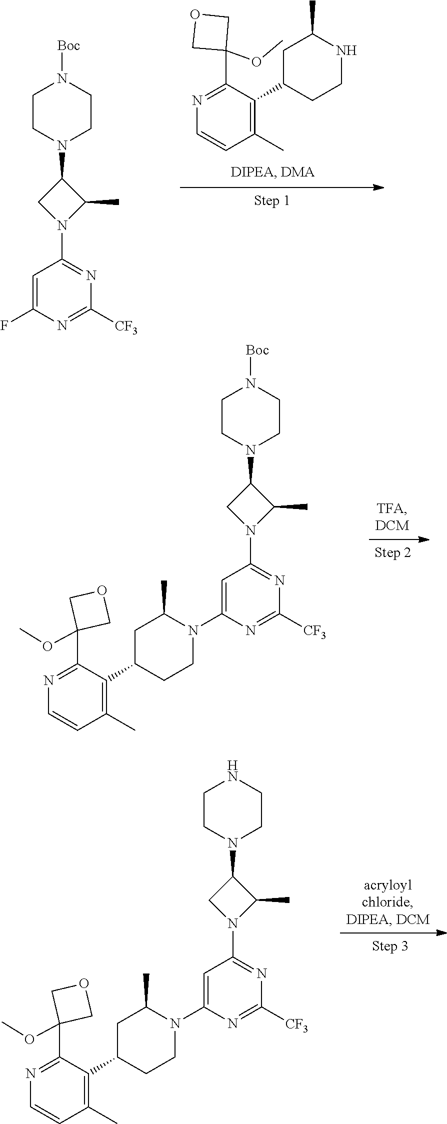

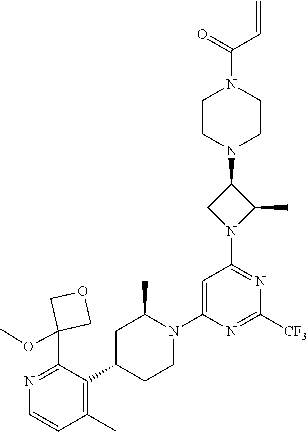 [SMILES: C=CC(=O)N1CCN([C@@H]2CN(c3cc(N4CC[C@H](c5c(C)ccnc5C5(OC)COC5)C[C@H]4C)nc(C(F)(F)F)n3)[C@@H]2C)CC1]

Step 1. To a stirred solution of tert-butyl 4-((2R,3R)-1-(6-fluoro-2-(trifluoromethyl)pyrimidin-4-yl)-2-methyl)azetidin-3-yl)piperazine-1-carboxylate (538 mg, 1.282 mmol) in DMA (8.00 mL) was added DIPEA (933 μL, 5.34 mmol. followed by 2-(3-methoxyoxetan-3-yl)-4-methyl-3-((2R,4S)-2-methylpiperidin-4-yl)pyridine (400 mg, 1.07 mmol). The resulting reaction mixture was stirred at 100° C. for 2 h. The reaction mixture was diluted with ice cold water and extracted with EtOAc (2×20 mL). The organic extracts were washed with brine solution (20 mL) and dried over Na₂SO₄. The solution was filtered and concentrated under reduced pressure. The crude material was purified on silica gel eluting with 80-100% EtOAc in pet ether to give tert-butyl 4-((2R,3R)-1-(6-((2R,4S)-4-(2-(3-methoxyoxetan-3-yl)-4-methylpyridin-3-yl)-2-methylpiperidin-1-yl)-2-(trifluoromethyl)pyrimidin-4-yl)-2-methyl)azetidin-3-yl)piperazine-1-carboxylate. m/z (ESI): 676.3 (M+H)⁺.

Step 2: To a vessel was added tert-butyl 4-((2R,3R)-1-(6-(2S,4R)-4-(2-(3-methoxyoxetan-3-yl)-4-methylpyridin-3-yl)-2-methylpiperidin-1-yl)-2-(trifluoromethyl)pyrimidin-4-yl)-2-methyl)azetidin-3-yl)piperazine-1-carboxylate (0.350 g. (0.518 mmol) and DCM (7 mL). The reaction mixture was cooled to 0° C., and then added TFA (0.399 mL, 5.18 mmol) was added. The reaction mixture was stirred at 0° C. for 2 h. The reaction mixture was concentrated to provide 4-((2S,4R)-4-(2-(3-methoxyoxetan-3-yl)-4-methylpyridin-3-yl)-2-methylpiperidin-1-yl)-6-((2R,3R)-2-methyl-3-(piperazin-1-yl)azetidin-1-yl)-2-(trifluoromethyl)pyrimidine which was carried forward to the next step.

Step 3: To a vessel was added 4-((2R,4S)-4-(2-(3-methoxyoxetan-3-yl)-4-methylpyridin-3-yl)-2-methylpiperidin-1-yl)-6-((2R,3R)-2-methyl-3-(piperazin-1-yl)azetidin-1-yl)-2-(trifluoromethyl)pyrimidine (350 mg, 0.520 mmol). DIPEA (454 μL, 2.60 mmol), and DCM (7.00 mL). The reaction mixture was cooled to −78° C., and acryloyl chloride (94 mg, 1.039 mmol) was added. The reaction mixture was stirred at −78° C. for 15 min. The reaction mixture was diluted with H₂O and extracted with DCM (3×10 mL), dried with Na₂SO₄, and concentrated under reduced pressure. The crude product was purified by prep. HPLC and lyophilized to give 1-(4-((2R,3R)-1-(6-((2R,4S)-4-(2-(3-methoxyoxetan-3-yl)-4-methylpyridin-3-yl)-2-methylpiperidin-1-yl)-2(trifluoromethyl)pyrimidin-4-yl)-2-methyl)azetidin-3-yl)piperazin-1-yl)prop-2-en-1-one. m/z (ESI): 630.4 (M+H)⁺. ¹H NMR (400 MHz, DMSO-d₆):): δ 8.30-8.29 (d, J=4.8 Hz, 1H), 7.19-7.17 (d, J=4.8 Hz, 1H), 6.83-6.72 (m, 1H), 6.13-6.08 (d, J=20 Hz, 1H), 5.70-5.67 (d, J=12.8 Hz, 1H), 5.57 (s, 1H), 5.21-5.14 (m, 2H), 4.85-4.79 (m, 2H), 4.51-4.47 (m, 1H), 3.94-3.87 (m, 2H), 3.59 (s, 4H), 3.33-3.25 (m, 1H), 3.02-2.96 (m, 5H), 2.73-2.49 (m, 5H), 2.29 (bs, 5H), 2.3-2.20 (m, 1H), 1.65-1.53 (d, J=48 Hz, 1H), 1.53-1.50 (d, J=48 Hz, 1H), 1.37-1.35 (d, J=8 Hz, 3H), 1.18-1.17 (t, J=4 Hz, 3H).

### Example 2-377: 1-(4-((2R,3R)-1-(6-((3S,4R)-4-(2-(3-methoxyoxetan-3-yl)-4-methylpyridin-3-yl)-3-methylpiperidin-1-yl)-2-(trifluoromethyl)pyrimidin-4-yl)-2-methyl)azetidin-3-yl)piperazin-1-yl)prop-2-en-1-one

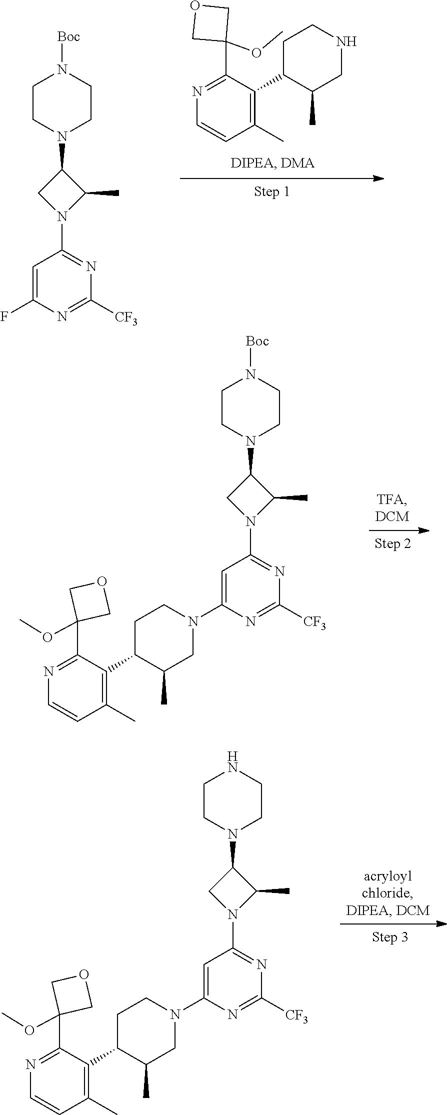

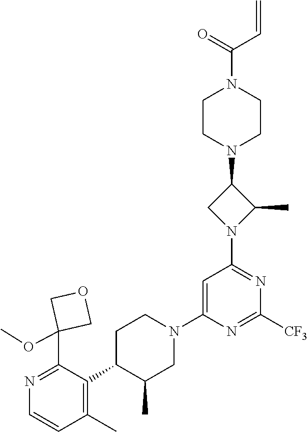 [SMILES: C=CC(=O)N1CCN([C@@H]2CN(c3cc(N4CC[C@@H](c5c(C)ccnc5C5(OC)COC5)[C@H](C)C4)nc(C(F)(F)F)n3)[C@@H]2C)CC1]

Step 1. To a stirred solution of tert-butyl 4-((2R,3R)-1-(6-fluoro-2-(trifluoromethyl)pyrimidin-4-yl)-2-methyl)azetidin-3-yl)piperazine-1-carboxylate (200 mg, 0.478 mmol) in DMA (2.20 mL) was added DIPEA (348 μL, 1.990 mmol) followed by tert-butyl 4-((2R,3R)-1-(6-fluoro-2-(trifluoromethyl)pyrimidin-4-yl)-2-methyl)azetidin-3-yl)piperazine-1-carboxylate (200 mg, 0.478 mmol). The resulted reaction mixture was stirred at 100° C. for 6 h. The reaction mixture was diluted with ice cold water and extracted with EtOAc (2×50 mL), the organic extracts were washed with brine solution (50 mL) and dried over Na₂SO₄. The solution was filtered and concentrated under reduced pressure. The crude material was purified on silica gel eluting with 80-100′% EtOAc in pet ether to give tert-butyl 4-((2R,3R)-1-(6-((3S,4R)-4-(2-(3-methoxyoxetan-3-yl)-4-methylpyridin-3-yl)-3-methylpiperidin-1-yl)-2-(trifluoromethyl)pyrimidin-4-yl)-2-methyl)azetidin-3-yl)piperazine-1-carboxylate. m/z (ESI): 676.3 (M+H)⁺.

Step 2: To a vessel was added tert-butyl 4-((2R,3R)-1-(6-((3S,4R)-4-(2-(3-methoxyoxetan-3-yl)-4-methylpyridin-3-yl)-3-methylpiperidin-1-yl)-2-(trifluoromethyl)pyrimidin-4-yl)-2-methyl)azetidin-3-yl)piperazine-1-carboxylate (200 mg, 0.296 mmol) and DCM (2.000 mL). The reaction mixture was cooled to 0° C. Then, TFA (22.8 μL, 0.296 mmol) was added at 0° C. The reaction mixture was stirred at 0° C.-25° C. for 2 h. The reaction mixture was concentrated to give 4-((3S,4R)-4-(2-(3-methoxyoxetan-3-yl)-4-methylpyridin-3-yl)-3-methylpiperidin-1-yl)-6-((2R,3R)-2-methyl-3-(piperazin-1-yl)azetidin-1-1)-2-(trifluoromethyl)pyrimidine. m/z (ESI): 576.3 (M+H)⁺

Step 3: To a vessel was added 4-((3S,4R)-4-(2-(3-methoxyoctan-3-yl)-4-methylpyridin-3-yl)-3-methylpiperidin-1-yl)-6-((2R,3R)-2-methyl-3-(piperazin-1-yl)azetidin-1-yl)-2-(trifluoromethyl)pyrimidine (190 mg, 0.282 mmol), DIPEA (246 μL, 1.41 mmol), and DCM (3.8 mL). The reaction mixture was cooled to −78° C., and acryloyl chloride (51.0 mg, 0.564 mmol) was added. The reaction mixture was stirred at −78° C. for 15 min. The mixture was diluted with H₂O and extracted with DCM (3×5 mL), dried with Na₂SO₄ concentrated under reduced pressure to get crude product, which was purified by prep. HPLC and lyophilized to provide 1-(4-((2R,3R)-1-(6-((3S,4R)-4-(2-(3-methoxyoxetan-3-yl)-4-methylpyridin-3-yl)-3-methylpiperidin-1-yl)-2-(trifluoromethyl)pyrimidin-4-yl)-2-methyl)azetidin-3-yl)piperazin-1-yl)prop-2-en-1-one. m/z (ESI): 630.3 (M+H)⁺. ¹H NMR (400 MHz, DMSO-d₆): δ (ppm) 8.29 (d, J=4.9 Hz, 1H), 7.18 (d, J=5.0 Hz, 1H), 6.81 (dd, J=16.7, 10.4 Hz, 1H), 6.11 (dd, J=16.7, 2.4 Hz, 1H), 5.74-5.57 (m, 2H), 5.14 (dd, J=11.2, 7.2 Hz, 2H), 4.79 (t, J=7.0 Hz, 2H), 4.57-4.36 (m, 3H), 3.96 (t, J=8.3 Hz, 1H), 3.87 (t, J=7.8 Hz, 1H), 3.59 (s, 3H), 3.25 (q, J=7.2 Hz, 1H), 2.95 (s, 3H), 2.83 (t, J=12.4 Hz, 2H), 2.43 (d, J=30.9 Hz, 1H), 2.37 (s, 4H), 2.29 (s, 5H), 2.01-1.84 (m, 1H), 1.66 (d, J=12.0 Hz, 1H), 1.40 (d, J=6.3 Hz, 3H), 0.66 (d, J=6.5 Hz, 3H).

### Example 2-378: 1-(4-((2R,3R)-2-methyl-1-(6-(2-methyl-4-(4-methyl-2-(2-oxa-6-azaspiro[3,3]heptan-6-yl)pyridin-3-yl)piperidin-1-yl)-2-(trifluoromethyl)pyrimidin-4-yl)azetidin-3-yl)piperazin-1-yl)prop-2-en-1-one

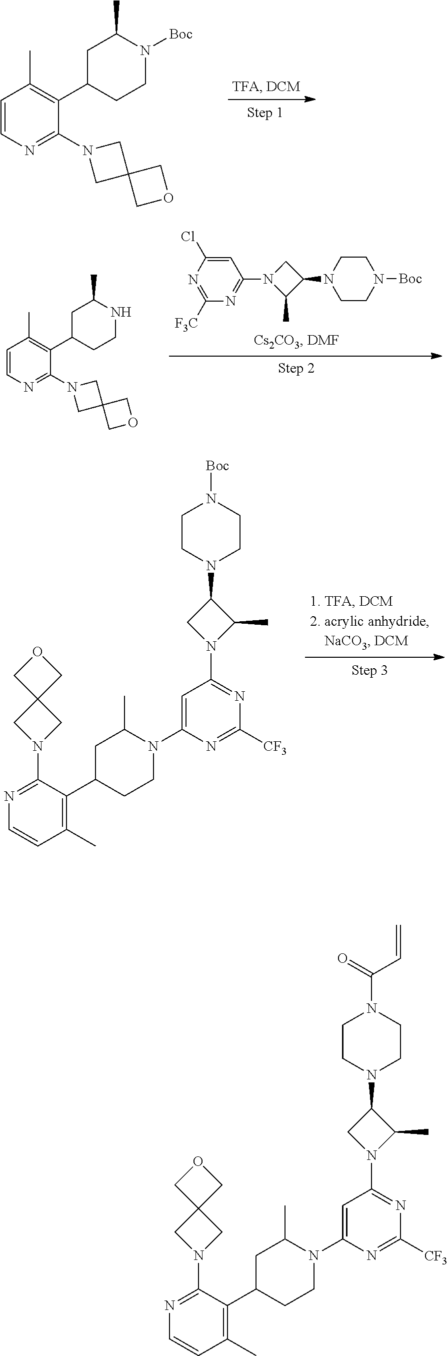

Step 1. To a solution of tert-butyl (2R)-2-methyl-4-(4-methyl-2-(2-oxa-6-azaspiro[3.3]heptan-6-yl)pyridin-3-yl)piperidine-1-carboxylate (400 mg, 1.032 mmol) in DCM (5 mL) was added TFA (1177 mg, 795 μL, 10.32 mmol, Sigma-Aldrich. Inc.) and the resulting mixture was stirred at rt for 15 min, then concentrated to afford 6-(4-methyl-3-((2R)-2-methylpiperidin-4-yl)pyridin-2-yl)-2-oxa-6-azaspiro[3.3]heptane TFA salt which was used as is. m/z (ESI): 288.2 (M+H)⁺.

Step 2, 6-(4-methyl-3-((2R)-2-methylpiperidin-4-yl)pyridin-2-yl)-2-oxa-6-azaspiro[3,3]heptane obtained as a TFA salt was dissolved in DMF (10 mL). To the reaction mixture was added tert-butyl 4-((2R,3R)-1-(6-fluoro-2-(trifluoromethyl)pyrimidin-4-yl)-2-methyl)azetidin-3-yl)piperazine-1-carboxylate (520 mg, 1.239 mmol) and Cs₂CO₃ (1682 mg, 5.16 mmol, Sigma-Aldrich, Inc.). The resulting mixture was heated at 120° C. for 24 h, then brought to rt. diluted with H₂O and extracted with EtOAc. The combined organics were dried over Na₂SO₄, filtered, concentrated and chromatographed on silica gel using 0-100% EtOAc in heptane to afford tert-butyl 4-((2R,3R)-2-methyl-1-(6-((2R)-2-methyl-4-(4-methyl-2-(2-oxa-6-azaspiro[3.3]heptan-6-yl)pyridin-3-yl)piperidin-1-yl)-2-(trifluoromethyl)pyrimidin-4-yl)azetidin-3-yl)piperazine-1-carboxylate. m/z (ESI): 587.3 (M+H-Boc)⁺.

Step 3. To a vessel was added tert-butyl 4-((2R,3R)-2-methyl-1-(6-((2R)-2-methyl-4-(4-methyl-2-(2-oxa-6-azaspiro[3.3]heptan-6-yl)pyridin-3-yl)piperidin-1-yl)-2-(trifluoromethyl)pyrimidin-4-yl)azetidin-3-yl)piperazine-1-carboxylate (22 mg, 0.032 mmol) and DCM (3 mL), which was followed by dropwise addition of TFA (0.119 mL, 1.602 mmol, Thermo Fisher Scientific). The reaction mixture was stirred at rt for 1 h to provide 6-(4-methyl-3-((2R,4S)-2-methyl-1-(6-((2R)-2-methyl-3-(piperazin-1-yl)azetidin-1-yl)-2-(trifluoromethyl)pyrimidin-4-yl)piperidin-4-yl)pyridin-2-yl)-2-oxa-6-azaspiro[3.3]heptane TFA salt as a solution in DCM which was directly used. m/z (ESI): 586.9 (M+H)⁺. To this DCM solution was added Na₂CO₃ (2 M in H₂O) (0.801 mL, 1.602 mmol, Sigma-Aldrich, Inc.) followed by acrylic anhydride (0.2 M in DCM) (0.160 mL, 0.032 mmol, Sigma-Aldrich, Inc.) at 0° C. The reaction was extracted using DCM (3×5 mL). The organic phase was dried through Na₂SO₄ and concentrated. The crude material was purified using Biotage Sfar HCD 10 g column with 0-7% MeOH in DCM to afford 1-(4-((2R)-2-methyl-1-(6-((2R)-2-methyl-4-(4-methyl-2-(2-oxa-6-azaspiro[3.3]heptan-6-yl)pyridin-3-yl)piperidin-1-yl)-2-(trifluoromethyl)pyrimidin-4-yl)azetidin-3-yl)piperazin-1-yl)prop-2-en-1-one. m/z (ESI): 640.8 (M+H)⁺. ¹H NMR (500 MHz, CHLOROFORM-d) δ ppm 7.97 (d, J=4.9 Hz, 1H), 6.55-6.63 (m, 2H), 6.29-6.35 (m, 1H), 5.73 (dd, J=10.5, 1.8 Hz, 1H), 5.25 (s, 1H), 4.87 (s, 4H), 4.49-4.62 (m, 1H), 4.27 (d, J=8.3 Hz, 2H), 4.18 (d, J=8.3 Hz, 2H), 3.89-4.08 (m, 3H), 3.76 (br s, 2H), 3.63 (br s, 2H), 3.28-3.35 (m, 2H), 3.08 (br s, 1H), 2.41-2.48 (m, 1H), 2.36-2.40 (m, 7H), 2.16 (br dd. J=12.8, 4.5 Hz, 1H), 1.72 (br d, J=13.2 Hz, 1H), 1.51 (d, J=6.4 Hz, 3H), 1.26-1.39 (m, 5H). ¹⁹F NMR (471 MHz, CHLOROFORM-d) δ ppm −71.74 (s, 1F).

The examples in the table below were prepared in a manner similar to that described above.

| | | | Pyrimidinyl | m/z (ESI): |
|---|---|---|---|---|
| Ex. | Chemical Structure | Name | halide | (M + H)⁺ |
| 2-379 | 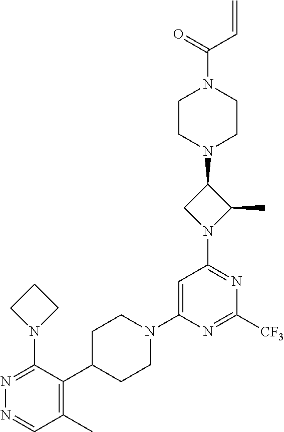 [SMILES: C=CC(=O)N1CCN([C@@H]2CN(c3cc(N4CCC(c5c(C)cnnc5N5CCC5)CC4)nc(C(F)(F)F)n3)[C@@H]2C)CC1] | 1-(4-((2R,3R)-1-(6-(4-(3- (1-azetidinyl)-5-methyl-4- pyridazinyl)-1-piperidinyl)- 2-(trifluoromethyl)-4- pyrimidinyl)-2-methyl-3- azetidinyl)-1-piperazinyl)- 2-propen-1-one | E-8 | 586.3 |
| 2-380 | 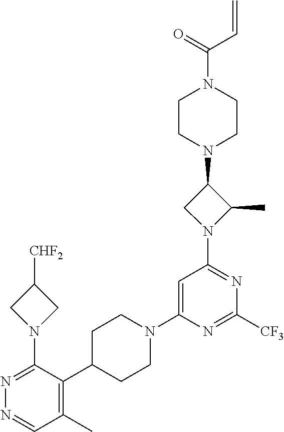 [SMILES: C=CC(=O)N1CCN([C@@H]2CN(c3cc(N4CCC(c5c(C)cnnc5N5CC(C(F)F)C5)CC4)nc(C(F)(F)F)n3)[C@@H]2C)CC1] | 1-(4-((2R,3R)-1~(6-(4-(3- (3-(difluoromethyl)-1- azetidinyl)-5-methyl-4- pyridazinyl)-1-piperidinyl)- 2-(trifluoromethyl)-4- pyrimidinyl)-2-methyl-3- azetidinyl)-1-piperazinyl)- 2-propen-1-one | E-8 | 636.4 |
| 2-382 | 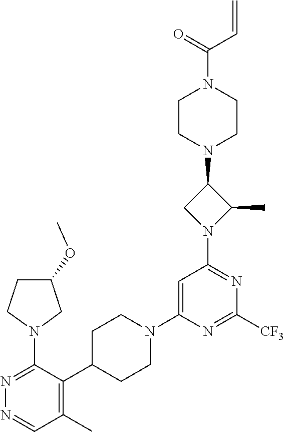 [SMILES: C=CC(=O)N1CCN([C@@H]2CN(c3cc(N4CCC(c5c(C)cnnc5N5CC[C@H](OC)C5)CC4)nc(C(F)(F)F)n3)[C@@H]2C)CC1] | 1-(4-((2R,3R)-1-(6-(4-(3- ((3S)-3-methoxy-1- pyrrolidinyl)-5-methyl-4- pyridazinyl)-1-piperidinyl)- 2-(trifluoromethyl)-4- pyrimidinyl)-2-methyl-3- azetidinyl)-1-piperazinyl)- 2-propen-1-one | E-8 | 630.4 |
| 2-383 | 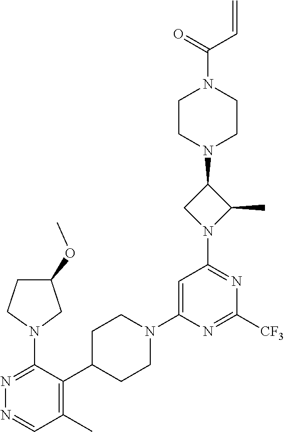 [SMILES: C=CC(=O)N1CCN([C@@H]2CN(c3cc(N4CCC(c5c(C)cnnc5N5CC[C@@H](OC)C5)CC4)nc(C(F)(F)F)n3)[C@@H]2C)CC1] | 1-(4-((2R,3R)-1-(6-(4-(3- ((3R)-3-methoxy-1- pyrrolidinyl)-5-methyl-4- pyridazinyl)-1-piperidinyl)- 2-(trifluoromethyl)-4- pyrimidinyl)-2-methyl-3- azetidinyl)-1-piperazinyl)- 2-propen-1-one | E-8 | 630.4 |
| 2-384 | 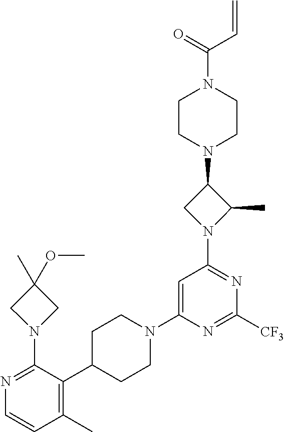 [SMILES: C=CC(=O)N1CCN([C@@H]2CN(c3cc(N4CCC(c5c(C)ccnc5N5CC(C)(OC)C5)CC4)nc(C(F)(F)F)n3)[C@@H]2C)CC1] | 1-(4-((2R,3R)-1-(6-(4-(2- (3-methoxy-3-methyl-1- azetidinyl)-4-methyl-3- pyridinyl)-1-piperidinyl)-2- (trifluoromethyl)-4- pyrimidinyl)-2-methyl~3- azetidinyl)-1-piperazinyl)- 2-propen-1-one | E-8 | 629.3 |
| 2-385 | 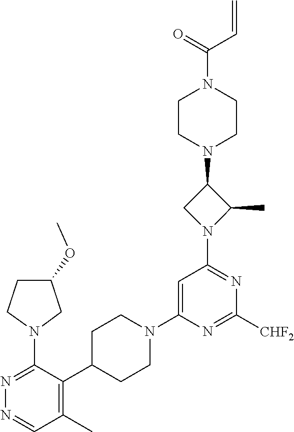 [SMILES: C=CC(=O)N1CCN([C@@H]2CN(c3cc(N4CCC(c5c(C)cnnc5N5CC[C@H](OC)C5)CC4)nc(C(F)F)n3)[C@@H]2C)CC1] | 1-(4-((2R,3R)-1-(2- (difluoromethyl)-6-(4-(3- ((3S)-3-methoxy-1- pyrrolidinyl)-5-methyl-4- pyridazinyl)-1-piperidinyl)- 4-pyrimidinyl)-2-methyl-3- azetidinyl)-1-piperazinyl)- 2-propen-1-one | E-9 | 612.4 |
| 2-386 | 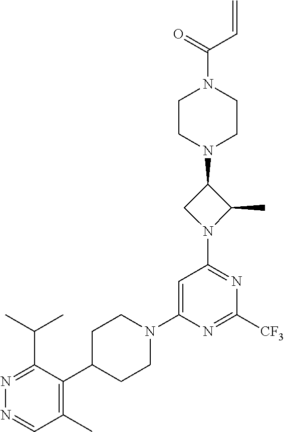 [SMILES: C=CC(=O)N1CCN([C@@H]2CN(c3cc(N4CCC(c5c(C)cnnc5C(C)C)CC4)nc(C(F)(F)F)n3)[C@@H]2C)CC1] | 1-(4-((2R,3R)-2-methyl-1- (6-(4-(5-methyl-3-(2- propanyl)-4-pyridazinyl)-1- piperidinyl)-2- (trifluoromethyl)-4- pyrimidinyl)-3-azetidinyl)- 1-piperazinyl)-2-propen-1- one | E-8 | 573.1 |
| 2-387 | 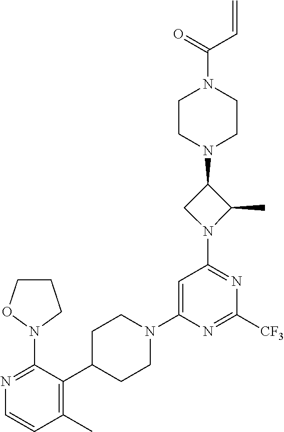 [SMILES: C=CC(=O)N1CCN([C@@H]2CN(c3cc(N4CCC(c5c(C)ccnc5N5CCCO5)CC4)nc(C(F)(F)F)n3)[C@@H]2C)CC1] | 1-(4-((2R,3R)-2-methyl-1- (6-(4-(4-methyl-2-(1,2- oxazolidin-2-yl)-3- pyridinyl)-1-piperidinyl)-2- (trifluoromethyl)-4- pyrimidinyl)-3-azetidinył)- 1-piperazinyl)-2-propen-1- one | E-8 | 600.9 |
| 2-388 | 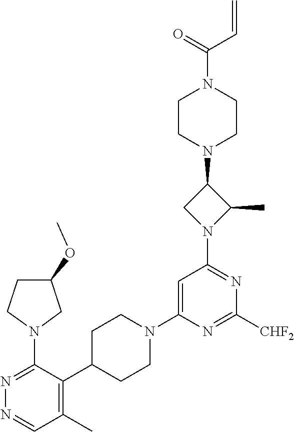 [SMILES: C=CC(=O)N1CCN([C@@H]2CN(c3cc(N4CCC(c5c(C)cnnc5N5CC[C@@H](OC)C5)CC4)nc(C(F)F)n3)[C@@H]2C)CC1] | 1-(4-((2R,3R)-1-(2- (difluoromethyl)-6-(4-(3- ((3R)-3-methoxy-1- pyrrolidinyl)-5-methyl-4- pyridazinyl)-1-piperidinyl)- 4-pyrimidinyl)-2-methyl-3- azetidinyl)-1-piperazinyl)- 2-propen-1-one | E-9 | 612.4 |
| 2-389 | 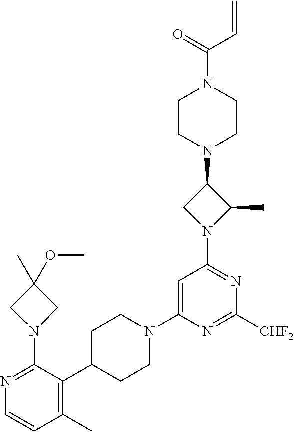 [SMILES: C=CC(=O)N1CCN([C@@H]2CN(c3cc(N4CCC(c5c(C)ccnc5N5CC(C)(OC)C5)CC4)nc(C(F)F)n3)[C@@H]2C)CC1] | 1-(4-((2R,3R)-1-(2- (difluoromethyl)-6-(4-(2- (3-methoxy-3-methyl-1- azetidinyl)-4-methyl-3- pyridinyl)-1-piperidinyl)-4- pyrimidinyl)-2-methyl-3- azetidinyl)-1-piperazinyl)- 2-propen-1-one | E-9 | 611.3 |
| 2-431 | 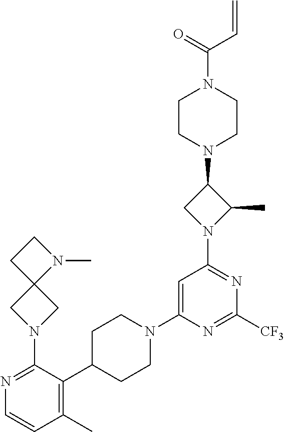 [SMILES: C=CC(=O)N1CCN([C@@H]2CN(c3cc(N4CCC(c5c(C)ccnc5N5CC6(CCN6C)C5)CC4)nc(C(F)(F)F)n3)[C@@H]2C)CC1] | 1-(4-((2R,3R)-2-methyl-1- (6-(4-(4-methyl-2-(1- methyl-1,6- diazaspiro[3.3]heptan-6- yl)-3-pyridinyl)-1- piperidinyl)-2- (trifluoromethyl)-4- pyrimidinyl)-3-azetidinyl)- 1-piperazinyl)-2-propen-1- one | E-8 | 640.0 |
| 2-644 | 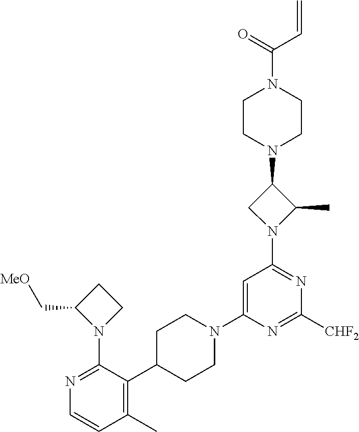 [SMILES: C=CC(=O)N1CCN([C@@H]2CN(c3cc(N4CCC(c5c(C)ccnc5N5CC[C@H]5COC)CC4)nc(C(F)F)n3)[C@@H]2C)CC1] | 1-(4-((2R,3R)-1-(2- (difluoromethyl)-6-(4-(2- ((S)-2- (methoxymethyl)azetidin-1- yl)-4-methylpyridin-3- yl)piperidin-1-yl)pyrimidin- 4-yl)-2-methylazetidin-3- yl)piperazin-1-yl)prop-2- en-1-one | E-9 | 611.4 |
| 2-645 | 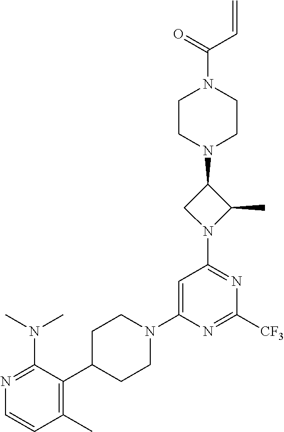 [SMILES: C=CC(=O)N1CCN([C@@H]2CN(c3cc(N4CCC(c5c(C)ccnc5N(C)C)CC4)nc(C(F)(F)F)n3)[C@@H]2C)CC1] | 1-(4-((2R,3R)-1-(6-(4-(2- (dimethylamino)-4- methylpyridin~3- yl)piperidin-1-yl)-2- (trifluoromethyl)pyrimidin- 4-yl)-2-methylazetidin-3- yl)piperazin-1-yl)prop-2- en-1-one | E-8 | 573.1 |
| 2-646 | 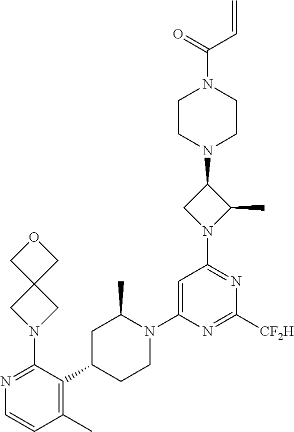 [SMILES: C=CC(=O)N1CCN([C@@H]2CN(c3cc(N4CC[C@H](c5c(C)ccnc5N5CC6(COC6)C5)C[C@H]4C)nc(C(F)F)n3)[C@@H]2C)CC1] | 1-(4-((2R,3R)-1-(2- (difluoromethyl)-6- ((2R,4S)-2-methyl-4-(4- methyl-2-(2-oxa-6- azaspiro[3.3]heptan-6- yl)pyridin-3-yl)piperidin-1- yl)pyrimidin-4-yl)-2- methylazetidin-3- yl)piperazin-1-yl)prop-2- en-1-one | E-9 | 623.2 |
| 2-647 | 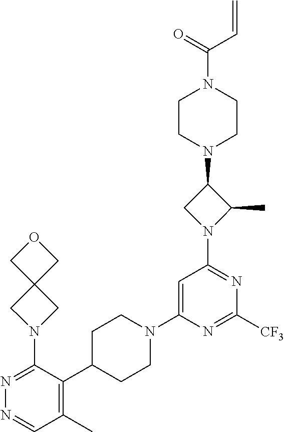 [SMILES: C=CC(=O)N1CCN([C@@H]2CN(c3cc(N4CCC(c5c(C)cnnc5N5CC6(COC6)C5)CC4)nc(C(F)(F)F)n3)[C@@H]2C)CC1] | 1-(4-((2R,3R)-2-methyl-1- (6-(4-(5-methyl-3-(2-oxa-6- azaspiro[3.3]heptan-6- yl)pyridazin-4-yl)piperidin- 1-yl)-2- (trifluoromethyl)pyrimidin- 4-vl)azetidin-3- yl)piperazin-1-yl)prop-2- en-1-one | E-8 | 628.8 |

### SFC Conditions for Chiral Separation

| | | | Racemic SM \| |
|---|---|---|---|
| Ex. # | Chemical Structure | Name | separation condition |
| 2-378-1 | 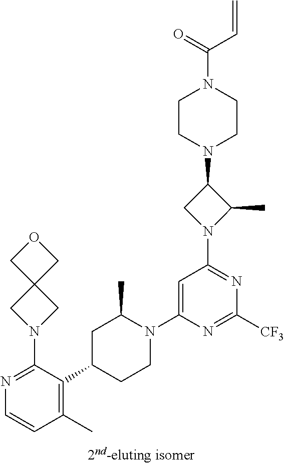 [SMILES: C=CC(=O)N1CCN([C@@H]2CN(c3cc(N4CC[C@H](c5c(C)ccnc5N5CC6(COC6)C5)C[C@H]4C)nc(C(F)(F)F)n3)[C@@H]2C)CC1] | 1-(4-((2R,3R)-2-methyl-1-(6- ((2R.4S)-2-mothyl-4-(4-mcthyl- 2-(2-oxa-6-azaspiro[3.3]heptan- 6-yl)pyridin-3-yl)piperidin-1-yl)- 2-(trifluoromethyl)pyrimidin-4- yl)azetidin-3-yl)piperazin-1- yl)prop-2-en-1-one | 2-378 \| Chiralpak IF, 250 × 21 mm 5 μm, column with a mobile phase of 45% MeOH in liquid CO₂ using a flow rate of 115 mL/min |
| 2-378-2 | 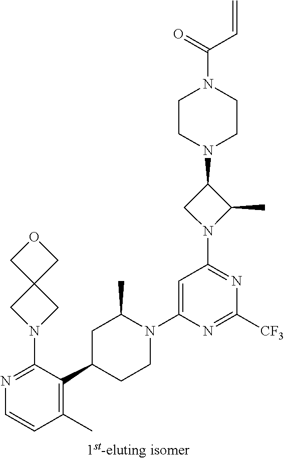 [SMILES: C=CC(=O)N1CCN([C@@H]2CN(c3cc(N4CC[C@@H](c5c(C)ccnc5N5CC6(COC6)C5)C[C@H]4C)nc(C(F)(F)F)n3)[C@@H]2C)CC1] | 1-(4-((2R,3R)-2-methyl-1-(6- ((2R,4R)-2-methyl-4-(4-methyl- 2-(2-oxa-6-azaspiro[3.3]heptan- 6-yl)-3-pyridinyl)-1-piperidinyl)- 2-(trifluoromethyl)-4- pyrimidinyl)-3-azetidinyl)-1- piperazinyl)-2-propen-1-one | 2-378 \| Chiralpak IF, 250 × 21 mm 5 μm, column with a mobile phase of 45% MeOH in liquid CO₂ using a flow rate of 115 mL/min |

### Example 2-392: 1-(4-((1R,3S) 3(6-((3S,4R)-4-(1-(2-methoxyethyl)-4-methyl-1H-pyrazol-5-yl)-3-methyl-1-piperidinyl)-2-(trifluoromethyl)-4-pyrindinyl)-2,2-dimethylcyclobutyl)-1-piperizinyl)-2-propen-1-one

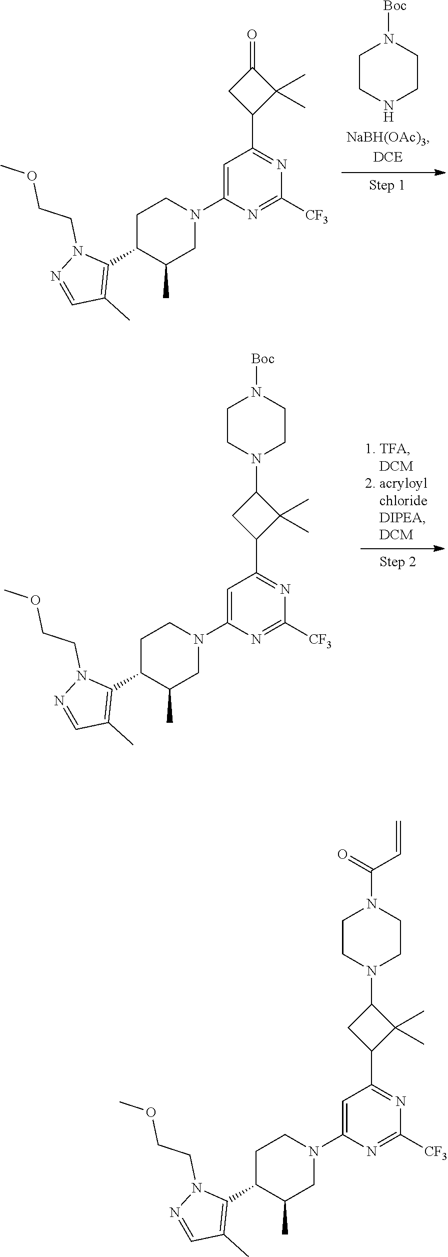 [SMILES: COCCn1ncc(C)c1[C@@H]1CCN(c2cc(C3CC(=O)C3(C)C)nc(C(F)(F)F)n2)C[C@H]1C ; CC(C)(C)OC(=O)N1CCNCC1 ; COCCn1ncc(C)c1[C@@H]1CCN(c2cc(C3CC(N4CCN(C(=O)OC(C)(C)C)CC4)C3(C)C)nc(C(F)(F)F)n2)C[C@H]1C ; C=CC(=O)N1CCN(C2CC(c3cc(N4CC[C@@H](c5c(C)cnn5CCOC)[C@H](C)C4)nc(C(F)(F)F)n3)C2(C)C)CC1]

Step 1. To a vessel was added 3-(6-((3S,4R)-4-(1-(2-methoxyethyl)-4-methyl-1H-pyrazol-5-yl)-3-methylpiperidin-1-yl)-2-(trifluoromethyl)pyrimidin-4-yl)-2,2-dimethylcyclobutane-1-one (53 mg, 0.111 mmol), tert-butyl piperazine-1-carboxylate (22.64 mg, 0.122 mmol, Combi-Blocks. Inc.), and 1,2-dichloroethane (1.1 mL). Sodium triacetoxyborohydride (35.1 mg, 0.166 mmol) mixture was stirred at rt for 18 h. The reaction was diluted with aqueous sat. NaHCO₃ (5 mL), extracted with DCM (3×10 mL), dried over MgSO₄, filtered, and concentrated. The crude material was absorbed onto a plug of silica gel and purified by chromatography through a Redi-Sep pre-packed silica gel column (40 g), eluting with a gradient of 0% to 100% EtOAc in heptane, to provide tert-butyl 4-(3-(6-(3S,4R)-4-(1-(2-methoxyethyl)-4-methyl-1H-pyrazol-5-yl)-3-methylpiperidin-1-yl)-2-(trifluoromethyl)pyrimidin-4-yl)-2,2-dimethylcyclobutyl)piperazine-1-carboxylate. m/z (ESI): 650.1 (M+H))⁺.

Step 2. To a vessel was added tert-butyl 4-(3-(6-((3S,4R)-4-(1-(2-methoxyethyl)-4-methyl-1H-pyrazol-5-yl)-3-methylpiperidin-1-yl)-2-(trifluoromethyl)pyrimidin-4-yl)-2,2-dimethylcyclobutyl)piperazine-1-carboxylate (68 mg, 0.105 mmol) and TFA (161 μL, 2.093 mmol, Apollo), and DCM (2 mL), and the mixture was stirred at 40° C. for 45 min. The reaction was concentrated in vacuo to give the crude 4-((1S,3R)-2,2-dimethyl-3-(piperazin-1-yl)cyclobutyl)-6-((3S,4R)-4-(1-(2-methoxyethyl)-4-methyl-1H-pyrazol-5-yl)-3-methylpiperidin-1-yl)-2-(trifluoromethyl)pyrimidine as a TFA salt, which was then redissolved in DCM (2 mL). To the reaction mixture was added DIPEA (135 mg, 183 μL, 1.046 mmol) and acryloyl chloride (9.35 μL, 0.115 mmol, Sigma-Aldrich, Inc.) and the reaction was left stirring at RT for 10 min. The reaction was diluted with aqueous sat. NaHCO₃ (10 mL). A small amount of solid Na₂CO₃ was added, and the reaction mixture was extracted with DCM (3×10 mL), dried over MgSO₄, filtered and concentrated. The crude material was absorbed onto a plug of silica gel and purified by chromatography through a Redi-Sep pre-packed silica gel column (40 g), eluting with a gradient of 0% to −100% 3:1 EtOAc:EtOH in heptane, to provide cis-1-(4-(3-(6-((3S,4R)-4-(1-(2-methoxy ethyl)-4-methyl-H-pyrazol-5-yl)-3-methylpiperidin-1-yl)-2-(trifluoromethyl)pyrimidin-4-yl)-2,2-dimethylcyclobutyl)piperazin-1-yl)prop-2-en-1-one 2,2,2-trifluoroacetate. m/z (ESI): 604.0 (M+H)⁺. ¹H NMR (400 MHz, MeOH-d₄) δ ppm 7.24 (s, 1H), 6.76 (dd, J=16.9, 10.7 Hz, 1H), 6.66 (s, 1H), 6.21 (dd, J=16.9, 1.9 Hz, 1H), 5.75 (dd, J=10.7, 1.9 Hz, 1H), 4.55-4.77 (m, 1H), 4.15-4.45 (m, 2H), 3.61-3.76 (m, 6H), 3.27-3.32 (m, 4H), 3.00-3.12 (m, 1H), 2.92-3.00 (m, 1H), 2.82-2.92 (m, 1H), 2.72 (br t, 1=12.4 Hz, 1H), 2.29-2.54 (m, 6H), 2.09-2.25 (m, 2H), 2.02 (s, 3H), 1.84-1.95 (m, 2H), 1.36 (s, 3H), 0.85 (dd, J=6.5, 1.7 Hz, 3H), 0.80 (s, 3H). ¹⁹F NMR (376 MHz, MeOH-d₄) δ ppm −72.86 (s, 3F).

The examples in the table below were prepared in a manner similar to that described above.

| | | | m/z (ESI): |
|---|---|---|---|
| Ex. | Chemical Structure | Name | (M + H)⁺ |
| 2-393 | 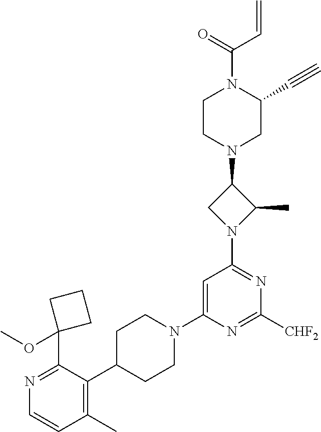 [SMILES: C#C[C@@H]1CN([C@@H]2CN(c3cc(N4CCC(c5c(C)ccnc5C5(OC)CCC5)CC4)nc(C(F)F)n3)[C@@H]2C)CCN1C(=O)C=C] | 1-((2R)-4-((2R,3R)-1-(2- (difluoromethyl)-6-(4-(2-(3- methoxy-3-oxetanyl)-4-methyl- 3-pyridinyl)-1-piperidinyl)-4- pyrimidinyl)-2-methyl-3- azetidinyl)-2-ethynyl-1- piperazinyl)-2-propen-1-one | 622.0 |
| 2-429 | 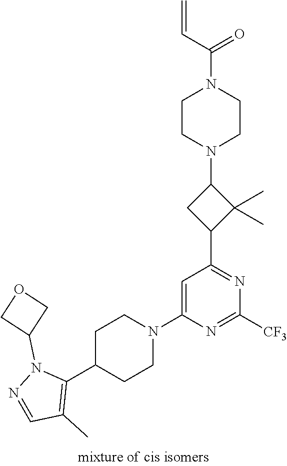 [SMILES: C=CC(=O)N1CCN(C2CC(c3cc(N4CCC(c5c(C)cnn5C5COC5)CC4)nc(C(F)(F)F)n3)C2(C)C)CC1] | 1-4-(2,2-dimethyl-3-(6-(4-(4- methyl-1-(3-oxetanyl)-1H- pyrazol-5-yl)-1-piperidinyl)-2- (trifluoromethyl)-4- pyrimidinyl)cyclobutyl)-1- piperazinyl)-2-propen-1-one | 588.3 |
| 2-477 | 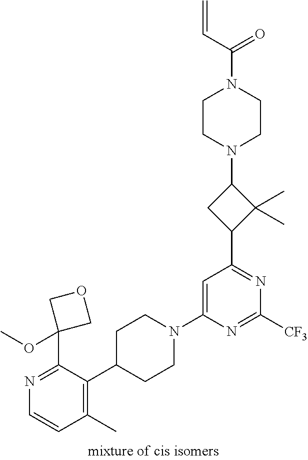 [SMILES: C=CC(=O)N1CCN(C2CC(c3cc(N4CCC(c5c(C)ccnc5C5(OC)COC5)CC4)nc(C(F)(F)F)n3)C2(C)C)CC1] | 1-(4-(-(3-(6-(4-(2-(3- methoxyoxetan-3-yl)-4- methylpyridin-3-yl)piperidin-1- yl)-2-(trifluoromethyl)pyrimidin- 4-yl)-2,2- dimethylcyclobutyl)piperazin-1- yl)prop-2-en-1-one | 629.4 |
| 2-478 | 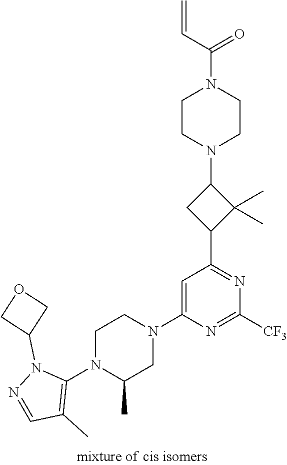 [SMILES: C=CC(=O)N1CCN(C2CC(c3cc(N4CCN(c5c(C)cnn5C5COC5)[C@H](C)C4)nc(C(F)(F)F)n3)C2(C)C)CC1] | 1-(4-(2,2-dimethyl-3-(6-((R)-3- methyl-4-(4-methyl-1-(oxetan-3- yl)-1H-pyrazol-5-yl)piperazin-1- yl)-2-(trifluoromethyl)pyrimidin- 4-yl)cyclobutyl)piperazin-1- yl)prop-2-en-1-one | 603.4 |
| 2-479 | 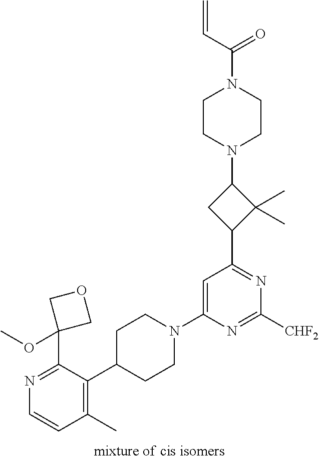 [SMILES: C=CC(=O)N1CCN(C2CC(c3cc(N4CCC(c5c(C)ccnc5C5(OC)COC5)CC4)nc(C(F)F)n3)C2(C)C)CC1] | 1-(4-(3-(2-(difluoromethyl)-6-(4- (2-(3-methoxyoxetan-3-yl)-4- methylpyridin-3-yl)piperidin-1- yl)pyrimidin-4-yl)-2,2- dimethylcyclobutyl)piperazin-1- yl)prop~2-en-1-one | 611.4 |
| 2-481 | 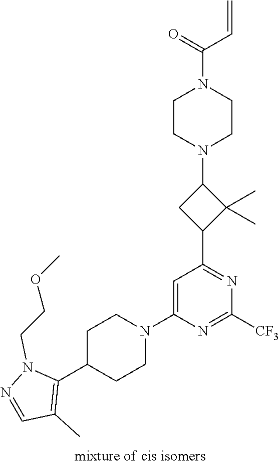 [SMILES: C=CC(=O)N1CCN(C2CC(c3cc(N4CCC(c5c(C)cnn5CCOC)CC4)nc(C(F)(F)F)n3)C2(C)C)CC1] | 1-(4-(3-(6-(4-(1-(2- methoxyethyl)-4-methyl-1H- pyrazol-5-yl)-1-piperidinyl)-2- (trifluoromethyl)-4-pyrimidinyl)- 2,2-dimethylcyclobutyl)-1- piperazinyl)-2-propen-1-one | 590.4 |
| 2-486 | 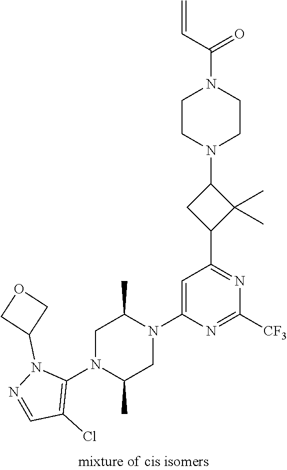 [SMILES: C=CC(=O)N1CCN(C2CC(c3cc(N4C[C@@H](C)N(c5c(Cl)cnn5C5COC5)C[C@H]4C)nc(C(F)(F)F)n3)C2(C)C)CC1] | 1-(4-(3-(6-((2R,5R)-4-(4-chloro- 1-(3-oxetanyl)-1H-pyrazol-5-yl)- 2,5-dimethyl-1-piperazinyl)-2- (trifluoromethyl)-4-pyrimidinyl)- 2,2-dimethylcyclobutyl)-1- piperazinyl)-2-propen-1-one | 637.3 |
| 2-488 | 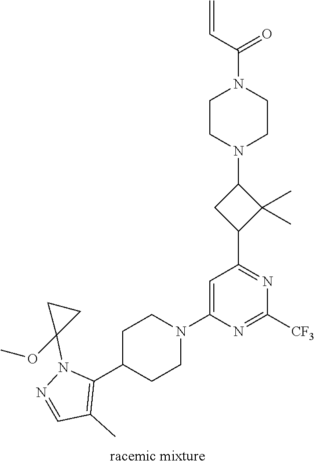 [SMILES: C=CC(=O)N1CCN(C2CC(c3cc(N4CCC(c5c(C)cnn5C5(OC)CC5)CC4)nc(C(F)(F)F)n3)C2(C)C)CC1] | 1-(4-(3-(6-(4-(1-(1- methoxycyclopropyl)-4-methyl- 1H-pyrazol-5-yl)-1-piperidinyl)- 2-(trifluoromethyl)-4- pyrimidinyl)-2.2- dimethylcyclobutyl)-1- piperazinyl)-2-propen-1-one | 602.4 |
| 2-514 | 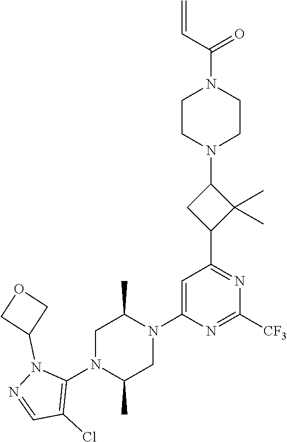 [SMILES: C=CC(=O)N1CCN(C2CC(c3cc(N4C[C@@H](C)N(c5c(Cl)cnn5C5COC5)C[C@H]4C)nc(C(F)(F)F)n3)C2(C)C)CC1] | 1-(4-(3-(6-((2R,5R)-4-(4-chloro- 1-(oxetan-3-yl)-1H-pyrazol-5- yl)-2,5-dimethylpiperazin-1-yl)- 2-(trifluoromethyl)pyrimidin-4- yl)-2,2- dimethylcyclobutyl)piperazin-1- yl)prop-2-en-1-one | 637.0 |
| 2-620 | 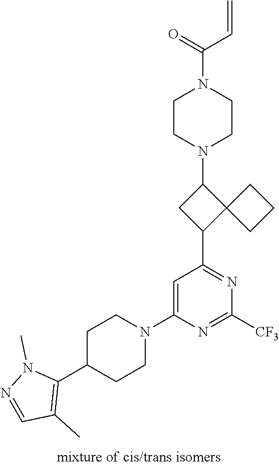 [SMILES: C=CC(=O)N1CCN(C2CC(c3cc(N4CCC(c5c(C)cnn5C)CC4)nc(C(F)(F)F)n3)C23CCC3)CC1] | 1-(4-(3-(6-(4-(1,4-dimethyl-1H- pyrazol-5-yl)-1-piperidinyl)-2- (trifluoromethyl)-4- pyrimidinyl)spiro[3.3]heptan-1- yl)-1-piperazinyl)-2-propen-1- one | 558.3 |
| 2-621 | 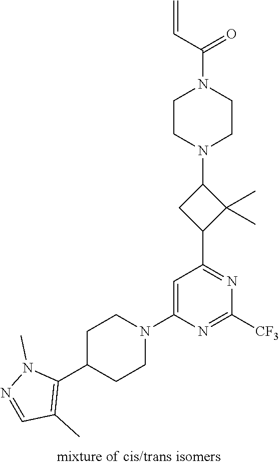 [SMILES: C=CC(=O)N1CCN(C2CC(c3cc(N4CCC(c5c(C)cnn5C)CC4)nc(C(F)(F)F)n3)C2(C)C)CC1] | 1-(4-(3-(6-(4-(1,4-dimethyl-1H- pyrazol-5-yl)-1-piperidinyl)-2- (trifluoromethyl)-4-pyrimidinyl)- 2,2-dimethylcyclobutyl)-1- piperazinyl)-2-propen-1-one | 546.3 |
| 2-480 | 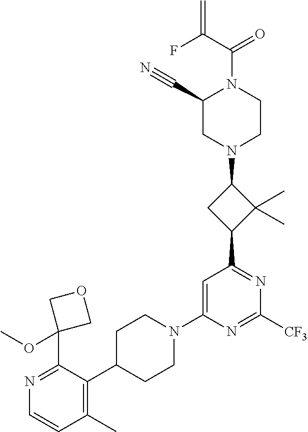 [SMILES: C=C(F)C(=O)N1CCN([C@@H]2C[C@H](c3cc(N4CCC(c5c(C)ccnc5C5(OC)COC5)CC4)nc(C(F)(F)F)n3)C2(C)C)C[C@H]1C#N] | (2S)-1-(2-fluoro-2-propenoyl)-4- ((1R.3S)-3-(6-(4-(2-(3-methoxy- 3-oxetanyl)-4-methyl-3- pyridinyl)-1-piperidinyl)-2- (trifluoromethyl)-4-pyrimidinyl)- 2,2-dimethylcyclobutyl)-2- piperazinecarbonitrile | 672.4 |

### SFC Conditions for Chiral Separation

| | | | Racemic SM \| |
|---|---|---|---|
| Ex. # | Chemical Structure | Name | separation condition |
| 2-392-1 | 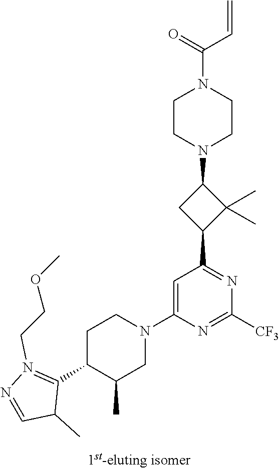 | 1-(4-((1R,3S)-3-(6-((3S,4R)-4- (1-(2-methoxyethyl)-4-methyl- 1H-pyrazol-5-yl)-3-methyl-1- piperidinyl)-2-(trifluoromethyl)- 4-pyrimidinyl)-2,2- dimethylcyclobutyl)-1- piperazinyl)-2-propen-1-one | 2-392 \| SFC Chiralpak ID, 21 × 250 mm 5 μm, column with a mobile phase of 40% 2-propanol using a flowrate of 90 mL/min |
| 2-477-1 | 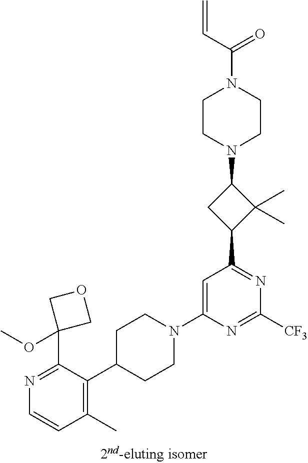 [SMILES: C=CC(=O)N1CCN([C@@H]2C[C@H](c3cc(N4CCC(c5c(C)ccnc5C5(OC)COC5)CC4)nc(C(F)(F)F)n3)C2(C)C)CC1] | 1-(4-(1R,3S)-3-(6-14-(2-(3- methoxy-3-oxetanyl)-4-methyl- 3-pyridinyl)-1-piperidinyl)-2- (trifluoromethyl)-4- pyrimidinyl)-2,2- dimethylcyclobutyl)-1- piperazinyl)-2-propen-1-one | 2-477 \| SFC Chiralpak ID, 2 × 25 cm 5 μm, column with a mobile phase of 45% IPA using a flow rate of 80 mL/min |
| 2-478-1 | 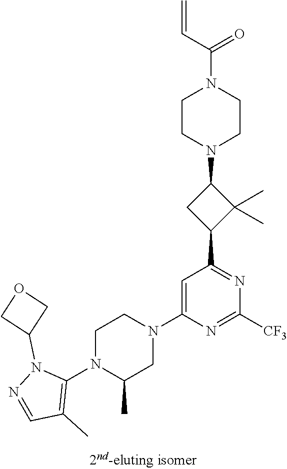 [SMILES: C=CC(=O)N1CCN([C@@H]2C[C@H](c3cc(N4CCN(c5c(C)cnn5C5COC5)[C@H](C)C4)nc(C(F)(F)F)n3)C2(C)C)CC1] | 1-(4-((1R,3S)-2,2-dimethyl-3- (6-(3R)-3-methyl-4-(4-methyl- 1-(3-oxetanyl)-1H-pyrazol-5- yl)-1-piperazinyl)-2- (trifluoromethyl)-4- pyrimidinyl)cyclobutyl)-1- piperazinyl)-2-propen-1-one | 2-478 \| SFC Chiralpak ID 2 × 25 cm 5 μm, column with a mobile phase of 45% IPA using a flow rate of 80 mL/min |
| 2-479-1 | 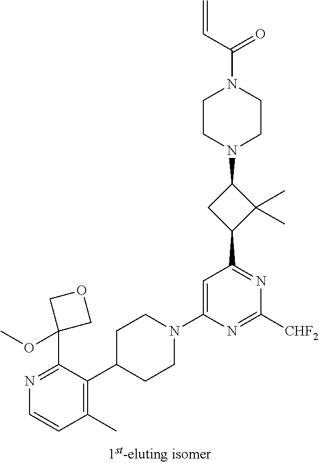 [SMILES: C=CC(=O)N1CCN([C@@H]2C[C@H](c3cc(N4CCC(c5c(C)ccnc5C5(OC)COC5)CC4)nc(C(F)F)n3)C2(C)C)CC1] | 1-(4-((1R,3S)-3-(2- (difluoromethyl)-6-(4-(2-(3- methoxy-3-oxetanyl)-4-methyl- 3-pyridinyl)-1-piperidinyl)-4- pyrimidinyl)-2,2- dimethylcyclobutyl)-1- piperazinyl)-2-propen-1-one | 2-479 \| SFC Chiralpak AS 2 × 25 cm 5 μm. column with a mobile phase of 45% MeOH (with 0.2% TEA) using a flow rate of 80 mL/min |
| 2-514-1 | 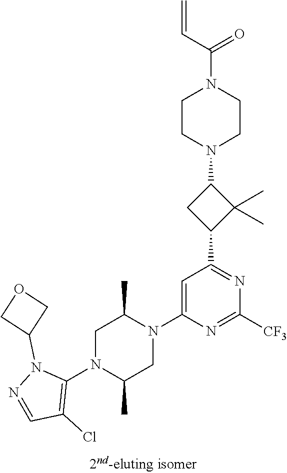 | 1-(4-((1S.3R)-3-(6-((2R,5R)-4- (4-chloro-1-(3-oxetany] )-1H- pyrazol-5-yl)-2,5-dimethyl-1- piperazinyl)-2- (trifluoromethyl)-4- pyrimidinyl)-2.2- dimethylcyclobutyl)-1- piperazinyl)-2-propen-1-one | 2-514 \| SFC Chiralpak ID 2 × 25 cm 5 μm, column with a mobile phase of 40% IPA using a flow rate of 80 mL/min |

### Example 2-399: 1-(4-((2R,3R)-1-(6-((2R,4S)-4-(2-(1,2-dimethoxy-2-propanyl)-4-methyl-3-pyridinyl)-2-methyl-1-piperidinyl)-2-(trifluoromethyl)-4-pyrimidinyl)-2-methyl-3-azetidinyl)-1-piperazinyl)-2-propen-1-one

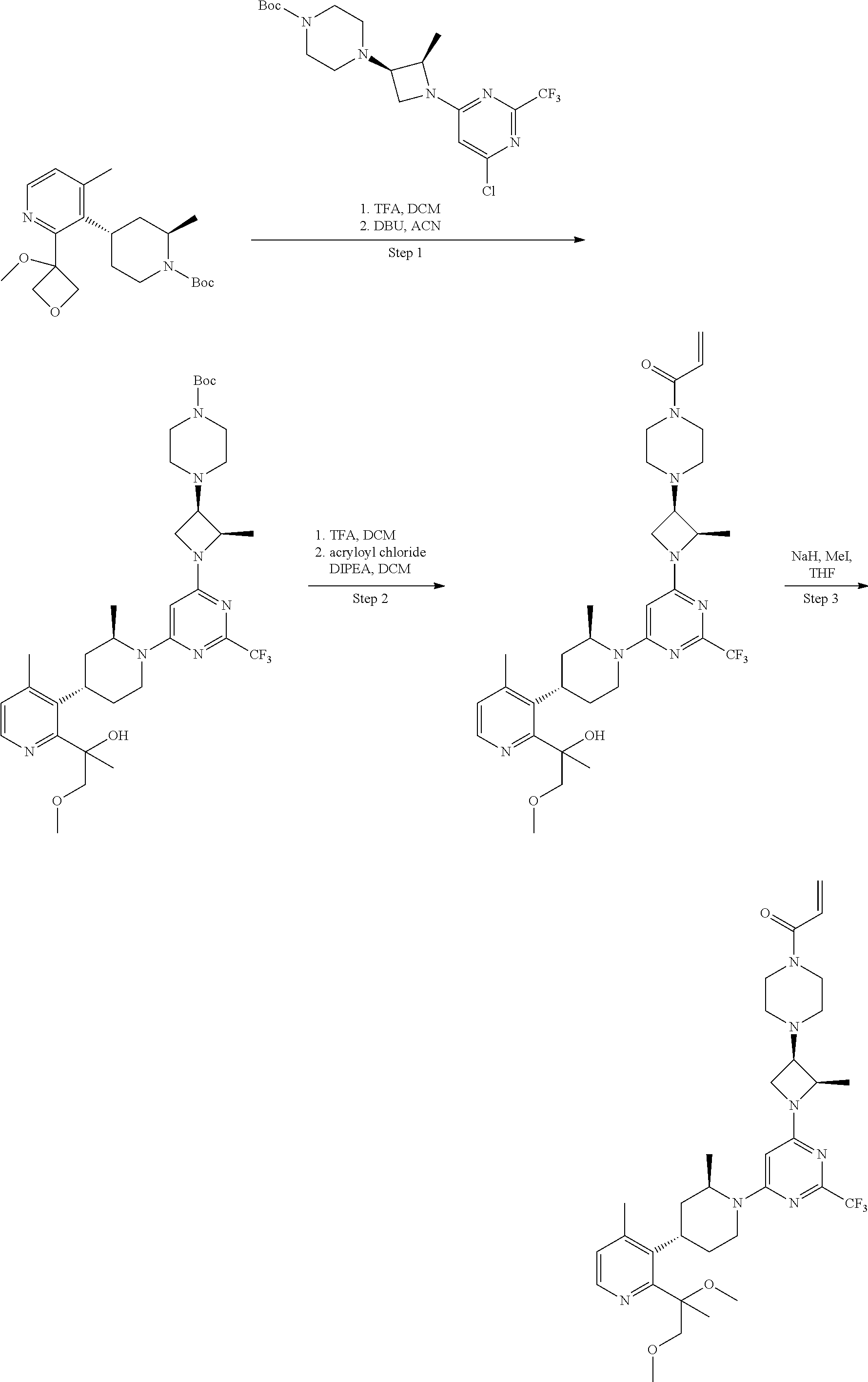 [SMILES: COC1(c2nccc(C)c2[C@H]2CCN(C(=O)OC(C)(C)C)[C@H](C)C2)COC1 ; C[C@@H]1[C@H](N2CCN(C(=O)OC(C)(C)C)CC2)CN1c1cc(Cl)nc(C(F)(F)F)n1 ; COCC(C)(O)c1nccc(C)c1[C@H]1CCN(c2cc(N3C[C@@H](N4CCN(C(=O)OC(C)(C)C)CC4)[C@H]3C)nc(C(F)(F)F)n2)[C@H](C)C1 ; C=CC(=O)N1CCN([C@@H]2CN(c3cc(N4CC[C@H](c5c(C)ccnc5C(C)(O)COC)C[C@H]4C)nc(C(F)(F)F)n3)[C@@H]2C)CC1 ; C=CC(=O)N1CCN([C@@H]2CN(c3cc(N4CC[C@H](c5c(C)ccnc5C(C)(COC)OC)C[C@H]4C)nc(C(F)(F)F)n3)[C@@H]2C)CC1]

Step 1. To a solution of tert-butyl 4-((2R,3R)-1-(6-chloro-2-(trifluoromethyl)pyrimidin-4-yl)-2-methyl)azetidin-3-yl)piperazine-1-carboxylate (0.659 g, 1.512 mmol) in DCM (3 mL) was added TFA (0.5 mL, 1.512 mmol, Sigma-Aldrich. Inc.) and the reaction mixture was stirred at rt for 30 min. The reaction mixture % as concentrated in vacuo and directly used without further purification. m/z (ESI): 277.2 (M+H)⁺.

To the above reaction vessel was added 1,8-diazabicyclo[5.4.0]undec-7-ene (1.611 mL, 10.58 mmol. Sigma-Aldrich, Inc.), tert-butyl 4-((2R,3R)-1-(6-chloro-2-(trifluoromethyl)pyrimidin-4-yl)-2-methyl)azetidin-3-yl)piperazine-1-carboxylate (0.659 g, 1.512 mmol), and ACN (3 mL) and the reaction mixture was heated to 80° C., and stirred for 24 h. The reaction mixture was diluted with H₂O and extracted with EtOAc. The organic extracts were washed with brine, filtered through a plug of Na₂SO₄. and concentrated in vacuo. The crude material was absorbed onto a plug of silica gel and purified by chromatography through a Redi-Sep pre-packed silica gel column, eluting with a gradient of 0% to 40% EtOAc in heptane, to provide tert-butyl 4-((2R,3R)-1-(6-((2R)-4-(2-(3-methoxyoxetan-3-yl)-4-methylpyridin-3-yl)-2-methylpiperidin-1-yl)-2-(trifluoromethyl)pyrimidin-4-yl)-2-methylazetidin-3-yl)piperazine-1-carboxylate. m/z (ESI): 676.0 (M+H)⁺.

Step 2. To a solution of tert-butyl 4-((2R,3R)-1-(6-((2R)-4-(2-(3-methoxyoxetan-3-yl)-4-methylpyridin-3-yl)-2-methylpiperidin-1-yl)-2-(trifluoromethyl)pyrimidin-4-yl)-2-methyl)azetidin-3-yl)piperazine-1-carboxylate (0.2588 g, 0.383 mmol) in DCM (3 mL) was added TFA (1 mL, 0.383 mmol, Sigma-Aldrich. Inc.) and the reaction mixture was stirred for 30 min at rt. The crude material was used as is. m/z (ESI): 576.0 (M+H)⁺.

The reaction mixture was concentrated in vacuo and redissolved in DCM (3 mL) and cooled to 0° C. To the reaction mixture was added DIPEA (0.667 mL, 3.83 mmol, Sigma-Aldrich, Inc.) and the reaction mixture was stirred for 5 min before acryloyl chloride (0.2 M in DCM) (1.915 mL, 0.383 mmol, Sigma-Aldrich, Inc.) was added and the reaction mixture was stirred at rt for 30 min. The reaction mixture was concentrated in vacuo, redissolved in EtOAc and washed with sat. aq. NaHCO₃ followed by aq. sat. NH₄Cl. The organic extracts were dried over a small plug of Na₂SO₄ and concentrated in vacuo. The crude material was purified by chromatography through a Redi-Sep pre-packed silica gel column, eluting with a gradient of 0% to 60% EtOAc/EtOH (3:1) in heptane. The material was further purified by reverse-phase preparative HPLC using 0.1% TFA in ACN/120, with a gradient of 10% to 100% over 15 min. The fractions containing product were combined and washed with sat. aq. Na₂CO₃ and extracted with EtOAc. The organic extracts were washed with brine, dried over Na₂SO₄, filtered and concentrated in vacuo to provide 1-(4-((2R,3R)-1-(6-((2R,4S)-4-(2-(3-methoxyoxetan-3-yl)-4-methylpyridin-3-yl)-2-methylpiperidin-1-yl)-2-(trifluoromethyl)pyrimidin-4-yl)-2-methyl)azetidin-3-yl)piperazin-1-yl)prop-2-en-1-one. ¹H NMR (400 MHz, CHLOROFORM-d) δ ppm 8.28-8.33 (m, 1H), 7.01-7.06 (m, 1H), 6.56 (dd, J=16.8, 10.6 Hz, 1H), 6.24-6.36 (m, 1H), 5.68-5.74 (m, 1H), 5.25-5.34 (m, 2H), 5.18-5.24 (m, 1H), 4.94 (t, J=6.1 Hz, 2H), 4.46-4.59 (m, 1H), 3.91-4.03 (m, 2H), 3.54-3.80 (m, 4H), 3.22-3.36 (m, 1H), 3.08-3.21 (m, 1H), 3.05-3.08 (m, 3H), 2.94-3.04 (m, 1H), 2.45-2.49 (m, 3H), 2.32-2.44 (m, 5H), 2.06-2.21 (m, 1H), 1.65-0.73 (m, 1H), 1.52-1.59 (m, 3H), 1.47-1.51 (m, 3H), 1.25-1.30 (m, 3H)).) ¹⁹F NMR (376 MHz, CHLOROFORM-d) δ ppm −71.77 (s, 3F) m/z (ESI): 630.0 (M+H)⁺.

Step 3. To a 0° C. suspension of sodium hydride (60% dispersion in mineral oil, 2.64 mg, 0.066 mmol, Oakwood Products, Inc.) in THF (I mL) was added a solution of 1-(4-((2R,3R)-1-(6-((2R,4S)-4-(2-(1-hydroxy-2-methoxypropan-2-yl)-4-methylpyridin-3-yl)-2-methylpiperidin-1-yl)-2-(trifluoromethyl)pyrimidin-4-yl)-2-methylazetidin-3-yl)piperazin-1-yl)prop-2-en-1-one (27.8 mg, 0.044 mmol) in THF (1 mL) and the reaction mixture was stirred for 15 min. To the reaction mixture was added iodomethane (0.027 mL, 0.044 mmol, Sigma-Aldrich. Inc.) and the reaction mixture was stirred at rt for 3 h. The reaction mixture was quenched by the slow addition of H₂O and extracted with EtOAc. The organic extracts were washed with brine, dried over Na₂SO₄, filtered, and concentrated in vacuo. The crude material was purified by chromatography through a Redi-Sep pre-packed silica gel column (4 g), eluting with a gradient of 0% to 40% EtOAc/EtOH (3:1) in heptane, to provide 1-(4-((2R,3R)-1-(6-((2R,4S)-4-(2-(1,2-dimethoxypropan-2-yl)-4-methylpyridin-3-yl)-2-methylpiperidin-1-yl)-2-(trifluoromethyl)pyrimidin-4-yl)-2-methyl)azetidin-3-yl)piperazin-1-yl)prop-2-en-1-one ¹H NMR (400 MHz, CHLOROFORM-d) δ ppm 8.19-8.27 (m, 1H), 6.90-7.00 (m, 1H), 6.49-6.63 (m, 1H), 6.24-6.38 (m, 1H), 5.64-5.81 (m, 1H), 5.17-5.26 (m, 1H), 4.67-4.84 (m, 1H), 4.49-4.59 (m, 1H), 4.07-4.22 (m, 1H), 3.91-4.02 (m, 2H), 3.80-3.87 (m, 1H), 3.55-3.79 (m, 4H), 3.48-3.53 (m, 3H), 3.24-3.32 (m, 1H), 3.15-3.20 (m, 3H), 3.03-3.12 (m, 1H), 2.42-2.53 (m, 4H), 2.37 (br t, J=4.9 Hz, 4H), 2.11-2.25 (m, 1H), 1.73-1.79 (m, 1H), 1.68-1.72 (m, 3H), 1.59-1.67 (m, 3H), 1.49 (d, J=6.1 Hz, 3H), 1.31-1.37 (m, 3H). ¹⁹F NMR (376 MHz, CHLOROFORM-d) δ ppm −71.76 (d, J=2.6 Hz, 3F) m/z (ESI): 646.0 (M+H)⁺

The example in the table below was prepared in a manner similar to that described above.

| | | | Pyridinyl | m/z (ESI): |
|---|---|---|---|---|
| Ex | Chemical Structure | Name | halide | (M + H)⁺ |
| 2-420 | 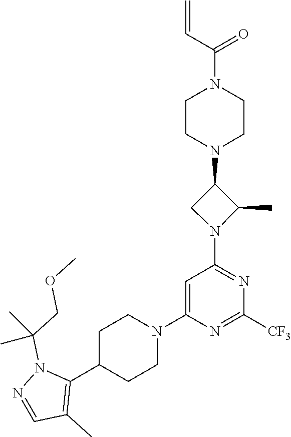 [SMILES: C=CC(=O)N1CCN([C@@H]2CN(c3cc(N4CCC(c5c(C)cnn5C(C)(C)COC)CC4)nc(C(F)(F)F)n3)[C@@H]2C)CC1] | 1-(4-((2R,3R)-1-(6-(4-(1-(1- methoxy-2-methyl-2-propanyl)- 4-methyl-1H-pyrazol-5-yl)-1- piperidinyl)-2-(trifluoromethyl)- 4-pyrimidinyl)-2-methyl-3- azetidinyl)-1-piperazinyl)-2- propen-1-one | E-1 | 605.1 |

### Example 2-473: (2E)-4,4-difluoro-1-((2R)-4-(1-(6-(4-(2-(3-methoxy-3-oxetanyl)-4-methyl-3-pyridinyl)-1-piperidinyl)-2-(trifluoromethyl)-4-pyrimidinyl)-3-azetidinyl)-2-methyl-1-piperazinyl)-2-buten-1-one

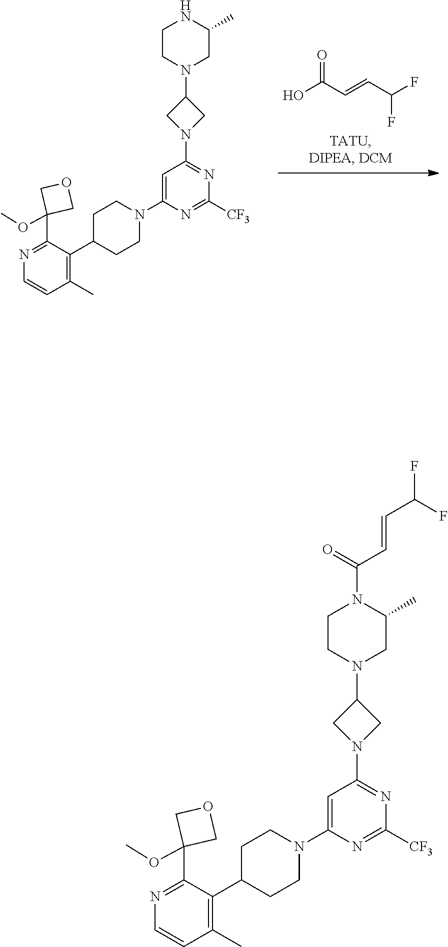 [SMILES: COC1(c2nccc(C)c2C2CCN(c3cc(N4CC(N5CCN[C@H](C)C5)C4)nc(C(F)(F)F)n3)CC2)COC1 ; O=C(O)/C=C/C(F)F ; COC1(c2nccc(C)c2C2CCN(c3cc(N4CC(N5CCN(C(=O)/C=C/C(F)F)[C@H](C)C5)C4)nc(C(F)(F)F)n3)CC2)COC1]

To a mixture of (R)-4-(4-(2-(3-methoxyoxetan-3-yl)-4-methylpyridin-3-yl)piperidin-1-yl)-6-(3-(3-methylpiperazin-1-Yl)azetidin-1-yl)-2-(trifluoromethyl)pyrimidine (83 mg, 0.148 mmol), DIPEA (57.3 mg, 0.077 mL, 0.443 mmol, Sigma-Aldrich. Inc.), and (2E)-4,4-difluorobut-2-enoic acid (21.65 mg, 0.177 mmol. Enamine) in DCM (1.5 mL) at 0° C. was added TATU (67.4 mg, 0.177 mmol, Oakwood Chemical). The resulting mixture was stirred at rt for 3 h. The mixture was then diluted with aq. saturated Na₂CO₃ (10 mL) and extracted with EtOAc (1×20 mL). The organic extract was washed with aq. saturated Na₂CO₃ (2×20 mL), dried over MgSO₄, and concentrated in vacuo. Chromatographic purification of the residue (silica gel, 0-100% EtOAc:EtOH (3:1)/heptane) provided (R,E)-4,4-difluoro-1-(4-(1-(6-(4-(2-(3-methoxyoxetan-3-yl)-4-methylpyridin-3-yl)piperidin-1-yl)-2-(trifluoromethyl)pyrimidin-4-yl)azetidin-3-yl)-2-methylpiperazin-1-yl)but-2-en-1-one. ¹H NMR (500 MHz, DMSO-d₆) δ ppm 8.29 (d, J=4.9 Hz, 1H), 7.17 (d, J=5.1 Hz, 1H), 7.06-7.15 (m, 1H), 6.44-6.73 (m, 2H), 5.68 (s, 1H), 5.15 (br d, J=7.1 Hz, 2H), 4.82 (d, J=7.3 Hz, 2H), 4.18-4.69 (m, 4H), 4.03-4.09 (m, 2H), 3.86 (br dd, J=9.0, 5.0 Hz, 1H), 3.76-3.82 (m, 1H), 3.21-3.29 (m, 2H), 2.96 (s, 3H), 2.83-2.91 (m, 3H), 2.67-2.78 (m, 2H), 2.37 (s, 3H), 1.97-2.11 (m, 3H), 1.76-1.92 (m, 1H), 1.61 (br dd, J=12.5, 1.8 Hz, 2H), 1.16-1.30 (m, 3H). m/z (ESI): 666.1 (M+H)⁺.

The examples in the table below were prepared in a manner similar to that described above.

| | | | Pyrimidinyl | m/z (ESI): |
|---|---|---|---|---|
| Ex. | Chemical Structure | Name | halide | (M + H)⁺ |
| 2-474 | 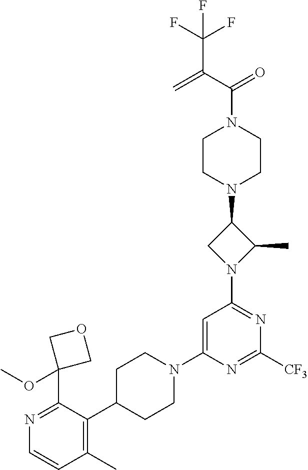 [SMILES: C=C(C(=O)N1CCN([C@@H]2CN(c3cc(N4CCC(c5c(C)ccnc5C5(OC)COC5)CC4)nc(C(F)(F)F)n3)[C@@H]2C)CC1)C(F)(F)F] | 1-(4-((2R,3R)-1-(6-(4-(2-(3- methoxy-3-oxetanyl)-4- methyl-3-pyridinyl)-1- piperidinyl)-2- (trifluoromethyl)-4- pyrimidinyl)-2-methyl-3- azetidinyl)-1-piperazinyl)-2- (trifluoromethyl)-2-propen-1- one | E-8 | 684.1 |
| 2-475 | 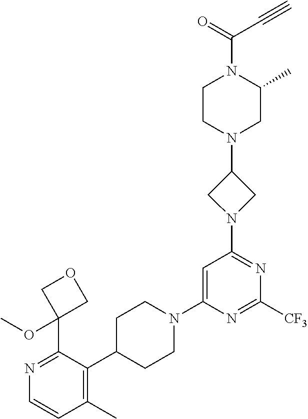 [SMILES: C#CC(=O)N1CCN(C2CN(c3cc(N4CCC(c5c(C)ccnc5C5(OC)COC5)CC4)nc(C(F)(F)F)n3)C2)C[C@H]1C] | 1-((2R)-4-(1-(6-(4-(2-(3- methoxy-3-oxetanyl)-4- methyl-3-pyridiny 1)-1- piperidinyl)-2- (trifluoromethyl)-4- pyrimidinyl)-3-azetidinyl)-2- methyl-1-piperazinyl)-2- propyn-1-one | E-4 | 614.1 |
| 2-476 | 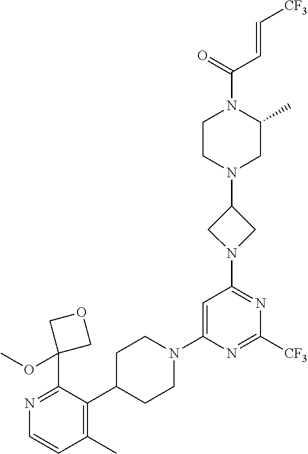 [SMILES: COC1(c2nccc(C)c2C2CCN(c3cc(N4CC(N5CCN(C(=O)/C=C/C(F)(F)F)[C@H](C)C5)C4)nc(C(F)(F)F)n3)CC2)COC1] | (2E)-4,4,4-trifluoro-1-((2R)-4- (1-(6-(4-(2-(3-methoxy-3- oxetanyl)-4-methyl-3- pyridinyl)-1-piperidinyl)-2- (trifluoromethyl)-4- pyrimidinyl)-3-azetidinyl)-2- methyl-1-piperazinyl)-2-buten- 1-one | E-4 | 684.0 |
| 2-485 | 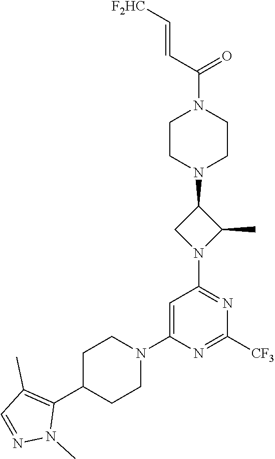 [SMILES: Cc1cnn(C)c1C1CCN(c2cc(N3C[C@@H](N4CCN(C(=O)/C=C/C(F)F)CC4)[C@H]3C)nc(C(F)(F)F)n2)CC1] | (2E)-1-(4-((2R,3R)-1-(6-(4- (1,4-dimethyl-1H-pyrazol-5- yl)-1-piperidinyl)-2- (trifluoromethyl)-4- pyrimidinyl)-2-methyl-3- azetidinyl)-1-piperazinyl)-4,4- difluoro-2-buten-1-one | E-1 | 583.3 |

### Example 2-622: 1-(4-((3R)-1-(6-(4-(4-Methyl-1-(3-oxetanyl)-1H-pyrazol-5-yl)-1-piperidinyl)-2-(trifluoromethyl)-4-pyrimidinyl)-6-oxa-1-azaspiro[3.3]heptan-3-yl)-1-piperidinyl)-2-propen-1-one

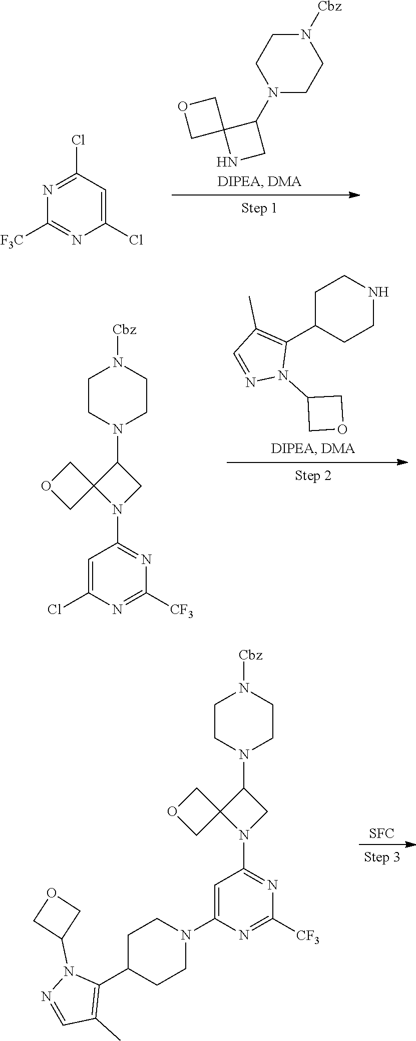 [SMILES: FC(F)(F)c1nc(Cl)cc(Cl)n1 ; O=C(OCc1ccccc1)N1CCN(C2CNC23COC3)CC1 ; O=C(OCc1ccccc1)N1CCN(C2CN(c3cc(Cl)nc(C(F)(F)F)n3)C23COC3)CC1 ; Cc1cnn(C2COC2)c1C1CCNCC1 ; Cc1cnn(C2COC2)c1C1CCN(c2cc(N3CC(N4CCN(C(=O)OCc5ccccc5)CC4)C34COC4)nc(C(F)(F)F)n2)CC1 ; "SFC"]

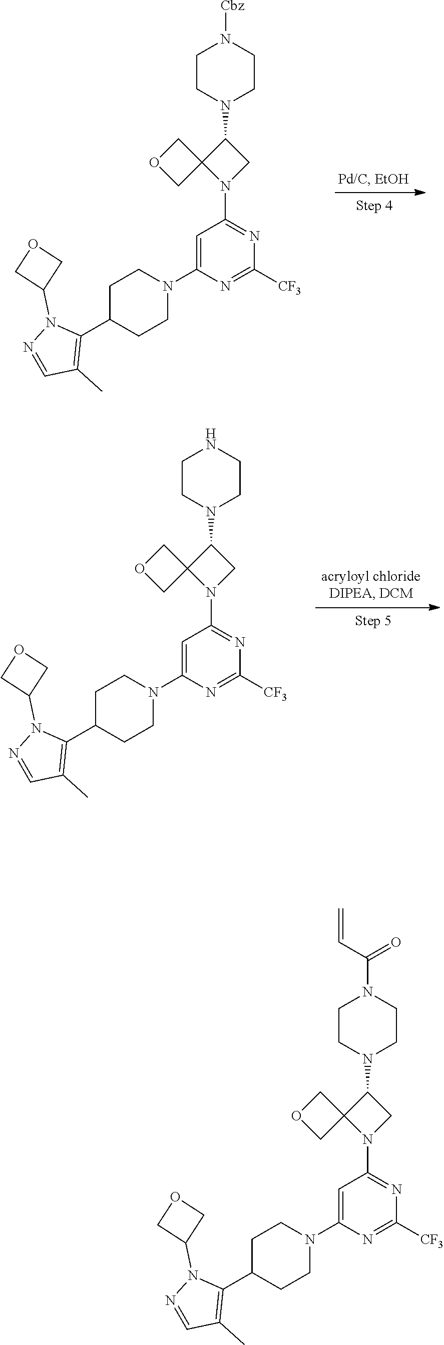 [SMILES: Cc1cnn(C2COC2)c1C1CCN(c2cc(N3C[C@@H](N4CCN(C(=O)OCc5ccccc5)CC4)C34COC4)nc(C(F)(F)F)n2)CC1 ; Cc1cnn(C2COC2)c1C1CCN(c2cc(N3C[C@@H](N4CCNCC4)C34COC4)nc(C(F)(F)F)n2)CC1 ; C=CC(=O)N1CCN([C@@H]2CN(c3cc(N4CCC(c5c(C)cnn5C5COC5)CC4)nc(C(F)(F)F)n3)C23COC3)CC1]

Step 1. To a vessel was added benzyl 4-(6-oxa-1-azaspiro[3.3]heptan-3-yl)piperazine-1-carboxylate (2 g, 4.81 mmol), 4,6-dichloro-2-(trifluoromethyl)pyrimidine (1.149 g, 5.30 mmol), and DMA (40.0 mL) and the reaction mixture was cooled to 0° C. DIPEA (4.20 mL, 24.07 mmol) was added slowly and the reaction mixture was allowed to stir at rt for 3 h. The reaction mixture was diluted with H₂O (50 mL) and extracted with EtOAc (2×50 mL). The organic extract was washed with satd. aq. NaCl (100 mL) and dried over Na₂SO₄. The solution was filtered, and concentrated in vacuo to give the crude material which was purified by chromatography through a Redi-Sep pre-packed silica gel column, eluting with a gradient of 20-50% EtOAc in hexanes, to provide benzyl 4-(1-(6-chloro-2-(trifluoromethylpyrimidin-4-yl)-6-oxa-1-azaspiro[3.3]heptan-3-yl)piperazine-1-carboxylate. m/z (ESI): 498.1 (M+H)⁺. ¹H NMR (400 MHz, DMSO-d₆) δ 7.46-7.27 (m, 5H), 6.77 (s, 1H), 5.44 (d, J=7.1 Hz, 1H), 5.10 (s, 3H), 4.70 (s, 1H), 4.47 (s, 1H), 3.99 (dd, J=17.2, 8.9 Hz, 1H), 3.85 (s, 1H), 3.47 (d, J=8.7 Hz, 5H), 2.50-−2.52 (m, 2H), 2.42 (s, 2H).

Step 2. To a vessel was added 2,2,2-trifluoro-1-(4-(4-methyl-1-(oxetan-3-yl)-1H-pyrazol-5-yl)-1H-piperidin-1-yl)ethan-1-one (1.227 g, 3.86 mmol) and DMA (24 mL) and the reaction mixture was cooled to 0° C. To the reaction mixture was slowly added DIPEA (2.81 mL, 16.07 mmol) and the reaction mixture was stirred at this temperature for 5 min. Then, benzyl 4-(1-(6-chloro-2-(trifluoromethyl)pyrimidin-4-yl)-6-oxa-1-azaspiro[3.3]heptan-3-yl)piperazine-1-carboxylate (1.6 g, 3.21 mmol) was added and the reaction mixture was heated to 110° C. for 16 h. The reaction mixture was diluted with H₂O (20 mL) and extracted with EtOAc (2×20 mL). The combined organic extracts were washed with satd. aq NaCl (25 mL) and dried over Na₂SO₄. The solution was filtered and concentrated in vacuo to give the crude material which was purified by chromatography through a Redi-Sep pre-packed silica gel column, eluting with a gradient of 0-4% MeOH in DCM, to give benzyl 4-(1-(6-(4-(4-methyl-1-(oxetan-3-yl)-1H-pyrazol-5-yl)piperidin-1-yl)-2-(trifluoromethyl)pyrimidin-4-yl)-6-oxa-1-azaspiro[3.3]heptan-3-yl)piperazine-1-carboxylate. m/z (ESI): 683.3 (M+H)⁺. ¹H NMR (400 MHz, DMSO-d₆) δ 7.44-7.23 (m, 5H), 5.83-5.60 (m, 2H), 5.36 (d, J=6.9 Hz, 1H), 5.11 (d, J=9.8 Hz, 3H), 4.97-−4.83 (m, 4H), 4.71 (d, J=7.1 Hz, 1H), 4.46 (d, J=23.5 Hz, 3H), 3.85 (t, J=7.9 Hz, 1H), 3.65 (t, J=7.3 Hz, 1H), 3.48 (s, 4H), 3.36 (d, J=6.5 Hz, 1H), 2.95 (s, 4H), 2.51-2.55 (m, 2H), 2.47-2.38 (m, 2H), 1.99 (d, J=1.5 Hz, 3H), 1.71 (d, J=8.7 Hz, 4H).

Step 3. The compound was purified by prep-SFC purification, using a Chiralpak IC, 150×50 mm 5μ, column with a mobile phase of 40% (1:1) ACN:MeOH in liquid CO₂ using a flowrate of 150 mL/min to afford:
Peak 1. benzyl (S)-4-(1-(6-(4-(4-methyl-1-(oxetan-3-yl)-1H-pyrazol-5-yl)piperidin-1-yl)-2-(trifluoromethyl)pyrimidin-4-yl)-6-oxa-1-azaspiro[3.3]heptan-3-yl)piperazine-1 carboxylate. m/z (ESI): 683.3 (M+H)⁺.
Peak 2. benzyl (R)-4-(1-(6-(4-(4-methyl-1-(oxetan-3-yl)-1H-pyrazol-5-yl)piperidin-1-yl)-2-(trifluoromethylpyrimidin-4-yl)-6-oxa-1-azaspiro[3.3]heptan-3-yl)piperazine-1-carboxylate. m/z (ESI): 683.3 (M+H)⁺.

Step 4. To a vessel was added (peak 2)benzyl (R)-4-(1-(6-(4-(4-methyl-1-(oxetan-3-yl)-1H-pyrazol-5-yl)piperidin-1-yl)-2-(trifluoromethyl)pyrimidin-4-yl)-6-oxa-1-azaspiro[3.3]heptan-3-yl)piperazine-1-carboxylate (0.4 g, 0.586 mmol) and EtOH (20 mL), 10°/o Pd/C (0.623 g, 0.586 mmol) was added and the mixture was degassed thoroughly and stirred in a bladder, under atmospheric hydrogen pressure for 2 h at RT. The mixture was filtered through a bed of celite, washed with MeOH, and concentrated in vacuo to pros ide (R)-1-(6-(4-(4-methyl-1-(oxetan-3-yl)-1H-pyrazol-5-yl)piperidin-1-yl)-2-(trifluoromethyl)pyrimidin-4-yl)-3-(piperazin-1-yl)-6-oxa-1-azaspiro[3.3]heptane, which was used without further purification. m/z (ESI): 549.3 (M+H)⁺.

Step 5. To a stirred solution of (R)-1-(6-(4-(4-methyl-1-(oxetan-3-yl)-1H-pyrazol-5-yl)piperidin-1-yl)-2-(trifluoromethyl)pyrimidin-4-yl)-3-(piperazin-1-yl)-6-oxa-1-azaspiro[3.3]heptane (0.29 g, 0.529 mmol) in DCM (14.5 mL) was added DIPEA (0.277 mL, 1.586 mmol) at −78° C., and the reaction mixture was stirred for 5 min. Then, acryloyl chloride (5.64 μL, 0.069 mmol) was added dropwise and stirred for 5 min at the same temperature. The reaction was quenched with ice cold water (10 mL) and extracted with DCM (3×20 mL). The combined organic extracts were washed with brine, dried over Na₂SO₄, and concentrated under reduced pressure to give the crude compound which was purified by prep-HPLC purification and lyophilized to obtain (R)-1-(4-(1-(6-(4-(4-methyl-1-(oxetan-3-yl)-1H-pyrazol-5-yl)piperidin-1-yl)-2-(trifluoromethyl)pyrimidin-4-yl)-6-oxa-1-azaspiro[3.3]heptan-3-yl)piperazin-1-yl)prop-2-en-1-one. m/z (ESI): 603.3 (M+H)⁺. ¹H NMR (400 MHz, DMSO-d₆) δ 7.30 (s, 1H), 6.83 (dd, J=16.7, 10.4 Hz, 1H), 6.13 (dd, J=16.7, 2.4 Hz, 1H), 5.83-5.62 (m, 3H), 5.37 (d, J=7.1 Hz, 1H), 5.13 (d, J=7.0 Hz, 1H), 4.93 (d, J=6.2 Hz, 2H), 4.91-4.83 (m, 2H), 4.73 (d, J=7.0 Hz, 1H), 4.50 (d, J=8.5 Hz, 3H), 3.86 (t J=7.9 Hz, 1H), 3.73-3.54 (m, 5H), 3.41-3.35 (m, 1H), 3.10-2.91 (m, 3H), 2.56 (s, 2H), 2.41 (dd, J=11.6, 5.6 Hz, 2H), 1.99 (s, 3H), 1.82-1.64 (m, 4H).

The examples in the table below were prepared in a manner similar to that described above.

| | | | m/z (ESI): |
|---|---|---|---|
| Ex. | Chemical Structure | Name | (M + H)⁺ |
| 2-362 | 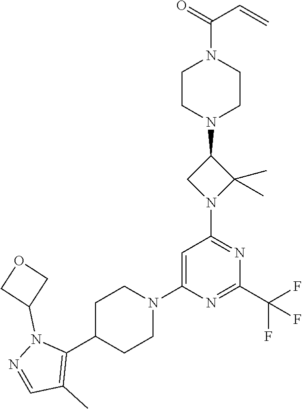 [SMILES: C=CC(=O)N1CCN([C@@H]2CN(c3cc(N4CCC(c5c(C)cnn5C5COC5)CC4)nc(C(F)(F)F)n3)C2(C)C)CC1] | 1-(4-((3R)-2,2- dimethyl-1-(6-(4-(4- methyl-1-(3-oxetanyl)- 1H-pyrazol-5-yl)-1- piperidinyl)-2- (trifluoromethyl)-4- pyrimidinyl)-3- azetidinyl)-1- piperazinyl)-2-propen-1- one | 589.3 |
| 2-363 | 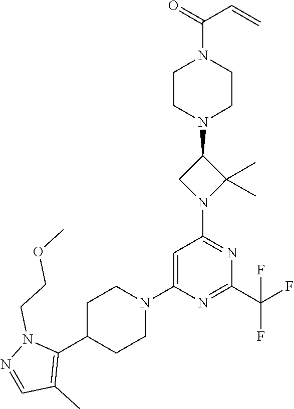 [SMILES: C=CC(=O)N1CCN([C@@H]2CN(c3cc(N4CCC(c5c(C)cnn5CCOC)CC4)nc(C(F)(F)F)n3)C2(C)C)CC1] | 1-(4-((3R)-1-(6-(4-(1- (2-methoxyethyl)-4- methyl-1H-pyrazol-5- yl)-1-piperidinyl)-2- (trifluoromethyl)-4- pyrimidinyl)-2,2- dimethyl-3-azetidinyl)- 1-piperazinyl)-2-propen- 1-one | 591.5 |
| 2-348 | 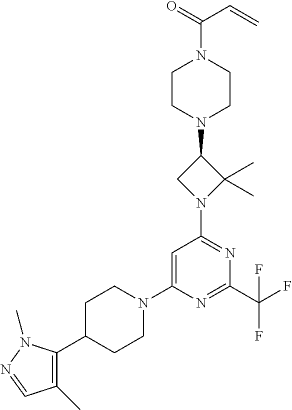 [SMILES: C=CC(=O)N1CCN([C@@H]2CN(c3cc(N4CCC(c5c(C)cnn5C)CC4)nc(C(F)(F)F)n3)C2(C)C)CC1] | 1-(4-((3R)-1-(6-(4-(1,4- dimethyl-1H-pyrazol-5- yl)-1-piperidinyl)-2- (trifluoromethy] )-4- pyrimidinyl)-2,2- dimethyl-3-azetidinyl)- 1-piperazinyl)-2-propen- 1-one | 547.3 |
| 2-345 | 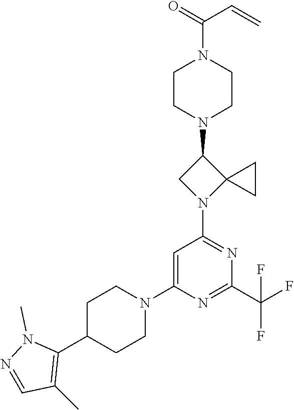 [SMILES: C=CC(=O)N1CCN([C@@H]2CN(c3cc(N4CCC(c5c(C)cnn5C)CC4)nc(C(F)(F)F)n3)C23CC3)CC1] | 1-(4-((6R)-4-(6-(4-(1,4- dimethyl-1H-pyrazol-5- yl)-1-piperidinyl)-2- (trifluoromethyl)-4- pyrimidinyl)-4- azaspiro[2.3]hexan-6- yl)-1-piperazinyl)-2- propen-1-one | 545.2 |

### Example 2-347: 1-(4-((2R,3R)-1-(6-((2R,4S)-4-(1-(2-methoxyethyl)-4-methyl-1H-pyrazol-5-yl)-2-methyl-1-piperidinyl)-2-(trifluoromethyl)-4-pyrimidinyl)-2-methyl-3-azetidinyl)-1-piperazinyl)-2-propen-1-one

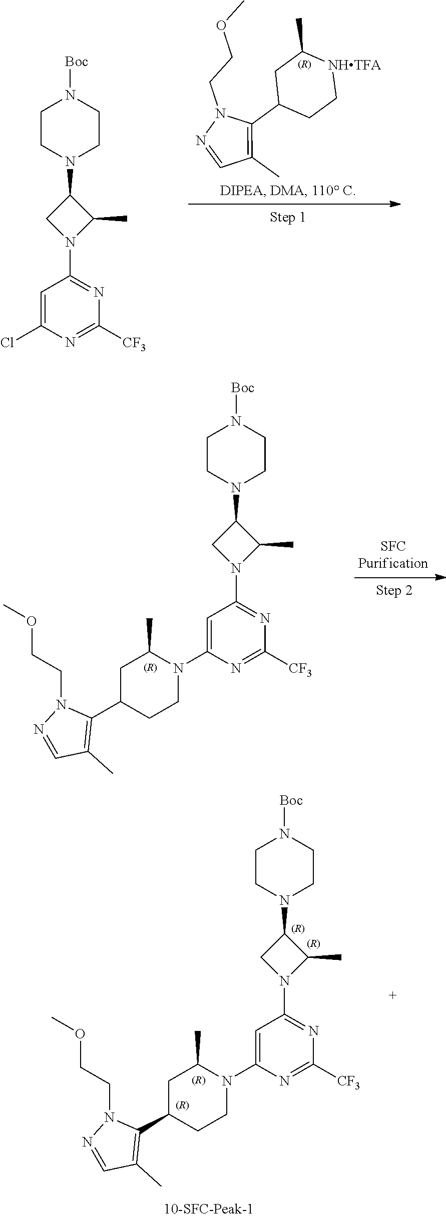

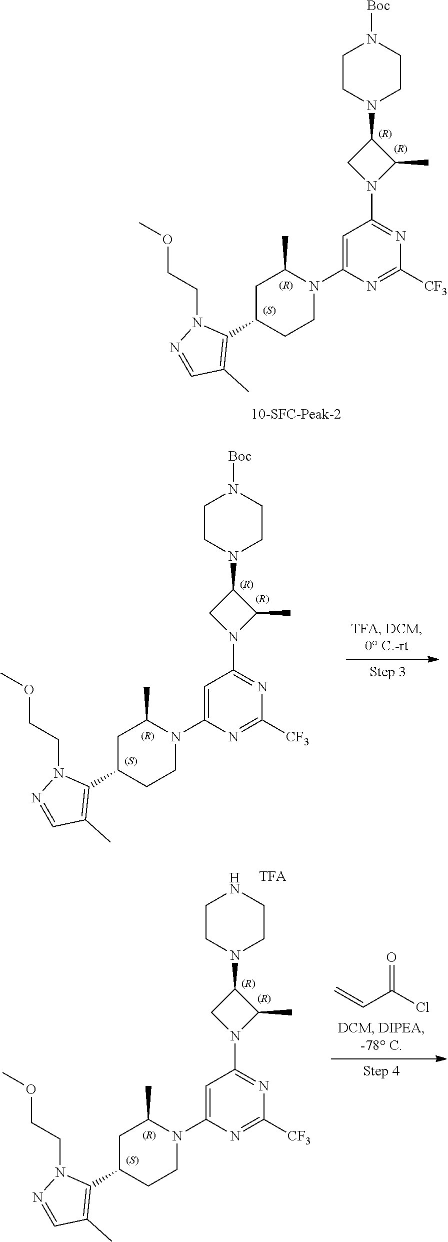

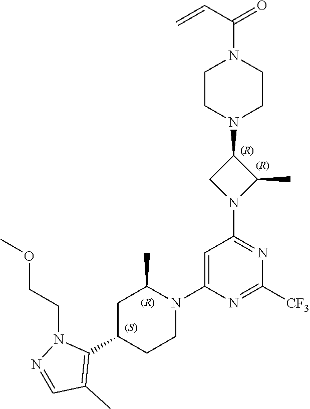

Step 1. To a stirred solution of 1-(2-methoxyethyl)-4-methyl-5-((3S)-3-methylcyclohexyl)-1H-pyrazole trifluoroacetate (3.4 g, 10.14 mmol) in DMA (68 mL) at 0° C. were added DIPEA (17.71 mL, 101 mmol) and tert-butyl 4-((2R,3R)-1-(6-chloro-2-(trifluoromethyl)pyrimidin-4-yl)-2-methyl)azetidin-3-yl)piperazine-1-carboxylate (4.86 g, 11.15 mmol) and stirred at 110° C. for 16 b. The reaction mixture was quenched with ice cold water (50 mL) and extracted with EtOAc (3×50 mL). The combined organic extracts were washed with water (50 mL) followed by brine (50 mL), dried (Na₂SO₄), filtered, and concentrated under reduced pressure. The crude material was purified by chromatography (Redi-sep pre-packed silica gel column (40 g), eluting with a gradient of 50-70% EtOAc in hexanes) to give tert-butyl 4-((2R,3R)-1-(6-((2R)-4-(1-(2-methoxyethyl)-4-methyl-1H-pyrazol-5-yl)-2-methylpiperidin-1-yl)-2-(trifluoromethyl)pyrimidin-4-yl)-2-methyl)azetidin-3-yl)piperazine-1-carboxylate (4 g, 6.28 mmol, 62% yield). m/z (ESI): 637.0 (M+H)⁺.

Step 2. tert-Butyl 4-((2R,3R)-1-(6-((2R)-4-(l-(2-methoxyethyl)-4-methyl-1H-pyrazol-5-yl)-2-methylpiperidin-1-yl)-2-(trifluoromethyl)pyrimidin-4-yl)-2-methyl)azetidin-3-yl)piperazine-1-carboxylate (4 g) was separated by SFC using an Amylose-SA (250×50 mm, 5μ) column with a mobile phase of liquid CO₂:methanol[85:15] using a flow rate of 150 mL/min to give:
Peak 1. tert-Butyl 4-((2R,3R)-1-(6-(2R,4R)-4-(1-(2-methoxyethyl)-4-methyl-1H-pyrazol-5-yl)-2-methylpiperidin-1-yl)-2-(trifluoromethyl)pyrimidin-4-yl)-2-methyl)azetidin-3-yl)piperazine-1-carboxylate. m/z (ESI): 637.0 (M+H)⁺.
Peak 2. tert-Butyl 4-((2R,3R)-1-(6-((2R,4S)-4-(1-(2-methoxyethyl)-4-methyl-1H-pyrazol-5-yl)-2-methylpiperidin-1-yl)-2-(trifluoromethyl)pyrimidin-4-yl)-2-methyl)azetidin-3-yl)piperazine-1-carboxylate. m/z (ESI): 637.0 (M+H)⁺. ¹H NMR (400 MHz, DMSO-d₆): δ 7.13 (s, 1H), 5.56 (s, 1H), 4.47 (q, J=6.6 Hz, 2H), 4.32-4.07 (m, 2H), 3.89 (dt, J=37.4, 8.4 Hz, 2H), 3.66-3.53 (m, 2H), 3.35 (m, 5H), 3.22 (m, 4H), 3.10-3.00 (m, 1H), 2.24 (m, 4H), 2.00 (m, 4H), 1.78 (m, 3H), 1.67 (d, J=12.8 Hz, 1H), 1.39 (d, J=7.6 Hz, 12H), 1.21 (d, J=6.8 Hz, 3H).

Step 3. To a stirred solution of tert-butyl 4-((2R,3R)-1-(6-((2R,4S)-4-(1-(2-methoxyethyl)-4-methyl-1H-pyrazol-5-yl)-2-methylpiperidin-1-yl)-2-(trifluoromethyl)pyrimidin-4-yl)-2-methyl)azetidin-3-yl)piperazine-1-carboxylate (peak 2, 300 mg, 0.471 mmol) in DCM (6 mL) at 0° C. was added TFA (726 μL, 9.42 mmol) dropwise and the reaction mass was stirred at rt for 1 h. The reaction mixture was concentrated under reduced pressure and the crude was triturated with diethyl ether (50 mL) to give 4-((2R,4S)-4-(1-(2-methoxyethyl)-4-methyl-1H-pyrazol-5-yl)-2-methylpiperidin-1-yl)-6-((2R,3R)-2-methyl-3-(piperazin-1-yl)azetidin-1-yl)-2-(trifluoromethyl)pyrimidine (290 mg, 0.457 mmol, 97% yield). m/z (ESI): 537.2 (M+H)⁺.

Step 4. To a stirred solution of 4-((2R,4S)-4-(1-(2-methoxyethyl)-4-methyl-1H-pyrazol-5-yl)-2-methylpiperidin-1-yl)-6-((2R,3R)-2-methyl-3-(piperazin-1-yl)azetidin-1-yl)-2-(trifluoromethyl)pyrimidine (300 mg, 0.473 mmol) in DCM (9 mL) at rt was added DIPEA (0.826 mL, 4.73 mmol) and stirred for 15 min. The reaction mixture was cooled to at −78° C., and acryloyl chloride (0.0573 mL, 0.709 mmol) was added. The reaction mixture was stirred at −78° C. for 15 min and quenched with ice cold water (10 mL). The reaction mixture was extracted with DCM (3×10 mL), the organic layer was washed with water (10 mL) followed by brine (30 mL), dried (Na₂SO₄), filtered and concentrated under reduced pressure. The crude material was purified by preparative HPLC purification (YMC Triart C-18 (250×20) mm, 5μ. column with a mobile phase of 0.1% NH₃ in water and ACN using a flow rate of 15 mL/min.) to give 1-(4-((2R,3R)-1-(6-((2R,4S)-4-(1-(2-methoxyethyl)-4-methyl-1H-pyrazol-5-yl)-2-methylpiperidin-1-yl)-2-(trifluoromethyl)pyrimidin-4-yl)-2-methyl)azetidin-3-yl)piperazin-1-yl)prop-2-en-1-one (82 mg, 0.139 mmol, 29% yield). m/z (ESI): 591.2 (M+H)⁺. ¹H NMR (400 MHz, DMSO-d₆) δ 7.13 (s, 1H), 6.81 (dd, J=16.7, 10.5 Hz, 1H), 6.11 (dd, J=16.7, 2.4 Hz, 1H), 5.69 (dd, J=10.4, 2.4 Hz, 1H), 5.57 (s, 1H), 4.49 (t, J=6.6 Hz, 1H), 4.31 (dt, J=14.5, 5.81 Hz, 1H), 4.19 (dt, J=14.6, 5.0 Hz, 1H), 3.95 (t, J=8.21 Hz, 1H), 3.87 (t, J=7.8 Hz, 1H), 3.69-3.48 (m, 6H), 3.38-3.33 (m, 2H), 3.23 (s, 5H), 3.11-2.96 (m, 1H), 2.29 (s, 4H), 2.01 (s, 4H), 1.79 (d, J=8.8 Hz, 2H), 1.67 (d, J=12.9 Hz, 1H), 1.40 (d, J=6.3 Hz, 3H), 1.21 (d, J=6.8 Hz, 3H).

The compounds listed in Table 2. below, have been synthesized by the methods previously described herein.

TABLE 2
ADDITIONAL COMPOUNDS OF THE DISCLOSURE

| | | | | m/z |
|---|---|---|---|---|
| Ex. | | | | (ESI): |
| # | Chemical Structure | Name | Core | (M + H)⁺ |
| 2-141 | 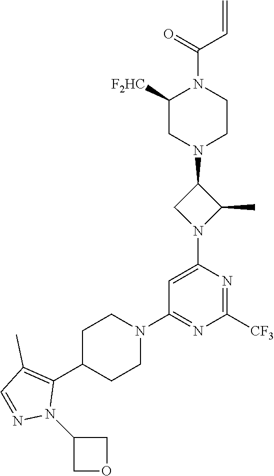 [SMILES: C=CC(=O)N1CCN([C@@H]2CN(c3cc(N4CCC(c5c(C)cnn5C5COC5)CC4)nc(C(F)(F)F)n3)[C@@H]2C)C[C@H]1C(F)F] | 1-((2S)-2- (difluoromethyl)-4- ((2R,3R)-2-methyl-1-(6- (4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-18 | 625.3 |
| 2-142 | 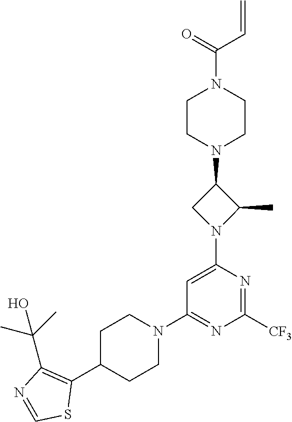 [SMILES: C=CC(=O)N1CCN([C@@H]2CN(c3cc(N4CCC(c5scnc5C(C)(C)O)CC4)nc(C(F)(F)F)n3)[C@@H]2C)CC1] | 1-(4-((2R,3R)-1-(6-(4- (4-(2-hydroxy-2- propanyl)-1,3-thiazol-5- yl)-1-piperidinyl)-2- (trifluoromethyl)-4- pyrimidinyl)-2-methyl- 3-azetidinyl)-1- piperazinyl)-2-propen-1- one | E-1 | 580.1 |
| 2-143 | 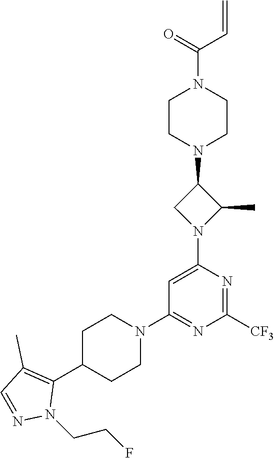 [SMILES: C=CC(=O)N1CCN([C@@H]2CN(c3cc(N4CCC(c5c(C)cnn5CCF)CC4)nc(C(F)(F)F)n3)[C@@H]2C)CC1] | 1-(4-((2R,3R)-1-(6-(4- (1-(2-fluoroethyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-8 | 565.1 |
| 2-144 | 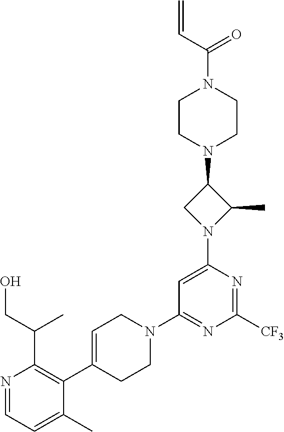 [SMILES: C=CC(=O)N1CCN([C@@H]2CN(c3cc(N4CC=C(c5c(C)ccnc5C(C)CO)CC4)nc(C(F)(F)F)n3)[C@@H]2C)CC1] | 1-(4-((2R,3R)-1-(6-(2- (1-hydroxy-2-propanyl)- 4-methyl-3′,6′- dihydro[3,4′-bipyridin]- 1′(2′H)-yl)-2- (trifluoromethyl)-4- pyrimidinyl)-2-methyl- 3-azetidinyl)-1- piperazinyl)-2-propen-1- one | E-1 | 586.2 |
| 2-145 | 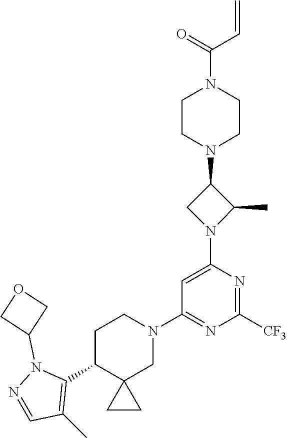 [SMILES: C=CC(=O)N1CCN([C@@H]2CN(c3cc(N4CC[C@@H](c5c(C)cnn5C5COC5)C5(CC5)C4)nc(C(F)(F)F)n3)[C@@H]2C)CC1] | 1-(4-((2R,3R)-2-methyl- 1-(6-((8R)-8-(4-methyl- 1-(3-oxetanyl)-1H- pyrazol-5-yl)-5- azaspiro[2.5]octan-5- yl)-2-(trifluoromethyl)- 4-pyrimidinyl)-3- azetidinyl)-1- piperazinyl)-2-propen-1- one | E-8 | 601.2 |
| 2-146 | 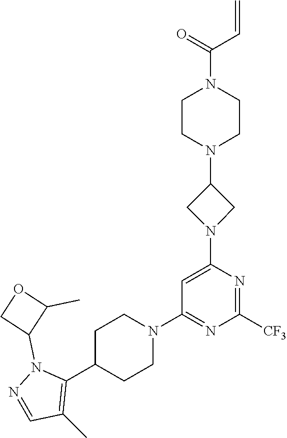 [SMILES: C=CC(=O)N1CCN(C2CN(c3cc(N4CCC(c5c(C)cnn5C5COC5C)CC4)nc(C(F)(F)F)n3)C2)CC1] | 1-(4-(1-(6-(4-(4-methyl- 1-(2-methyl-3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-3 | 574.7 |
| 2-147 | 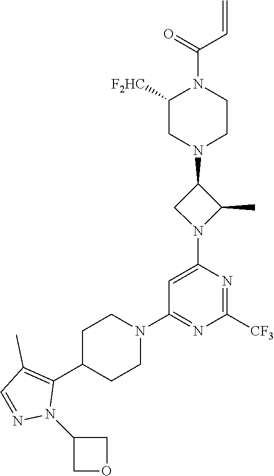 [SMILES: C=CC(=O)N1CCN([C@@H]2CN(c3cc(N4CCC(c5c(C)cnn5C5COC5)CC4)nc(C(F)(F)F)n3)[C@@H]2C)C[C@@H]1C(F)F] | 1-((2R)-2- (difluoromethyl)-4- ((2R,3R)-2-methyl-1-(6- (4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-18 | 625.3 |
| 2-148 | 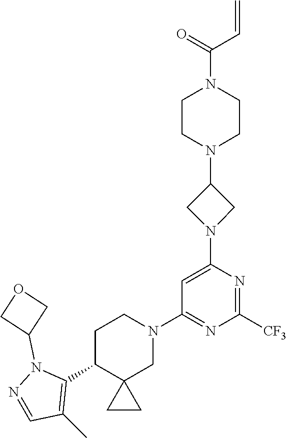 [SMILES: C=CC(=O)N1CCN(C2CN(c3cc(N4CC[C@@H](c5c(C)cnn5C5COC5)C5(CC5)C4)nc(C(F)(F)F)n3)C2)CC1] | 1-(4-(1-(6-((8R)-8-(4- methyl-1-(3-oxetanyl)- 1H-pyrazol-5-yl)-5- azaspiro[2.5]octan-5- yl)-2-(trifluoromethyl)- 4-pyrimidinyl)-3- azetidinyl)-1- piperazinyl)-2-propen-1- one | E-3 | 587.3 |
| 2-151 | 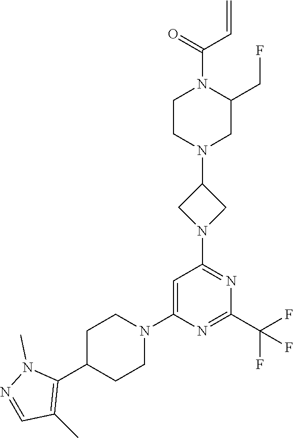 [SMILES: C=CC(=O)N1CCN(C2CN(c3cc(N4CCC(c5c(C)cnn5C)CC4)nc(C(F)(F)F)n3)C2)CC1CF] | 1-(4-(1-(6-(4-(1,4- dimethyl-1H-pyrazol-5- yl)-1-piperidinyl)-2- (trifluoromethyl)-4- pyrimidinyl)-3- azetidinyl)-2- (fluoromethyl)-1- piperazinyl)-2-propen-1- one | E-16 | 551.3 |
| 2-152 | 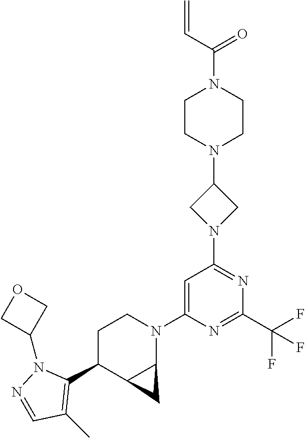 [SMILES: C=CC(=O)N1CCN(C2CN(c3cc(N4CC[C@H](c5c(C)cnn5C5COC5)[C@@H]5C[C@@H]54)nc(C(F)(F)F)n3)C2)CC1] | 1-(4-(1-(6-((1S,5S,6S)- 5-(4-methyl-1-(3- oxetanyl)-1H-pyrazol-5- yl)-2- azabicyclo[4.1.0]heptan- 2-yl)-2- (trifluoromethyl)-4- pyrimidinyl)-3- azetidinyl)-1- piperazinyl)-2-propen-1- one | E-3 | 572.7 |
| 2-153 | 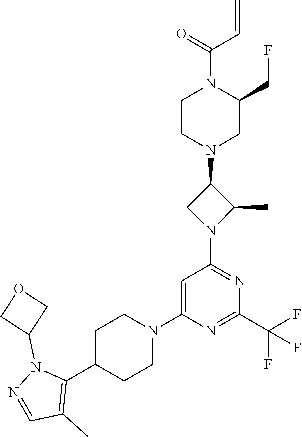 [SMILES: C=CC(=O)N1CCN([C@@H]2CN(c3cc(N4CCC(c5c(C)cnn5C5COC5)CC4)nc(C(F)(F)F)n3)[C@@H]2C)C[C@@H]1CF] | 1-((2R)-2- (fluoromethyl)-4- ((2R,3R)-2-methyl-1-(6- (4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-17 | 607.3 |
| 2-154 | 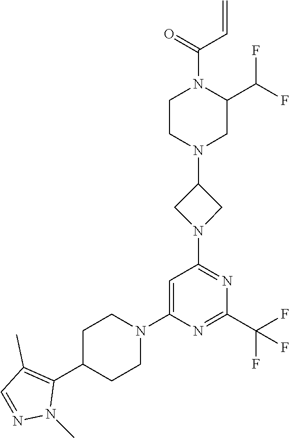 [SMILES: C=CC(=O)N1CCN(C2CN(c3cc(N4CCC(c5c(C)cnn5C)CC4)nc(C(F)(F)F)n3)C2)CC1C(F)F] | 1-(2-(difluoromethyl)-4- (1-(6-(4-(1,4-dimethyl- 1H-pyrazol-5-yl)-1- piperidinyl)-2- (trifluoromethyl)-4- pyrimidinyl)-3- azetidinyl)-1- piperazinyl)-2-propen-1- one | E-7 | 569.2 |
| 2-155 | 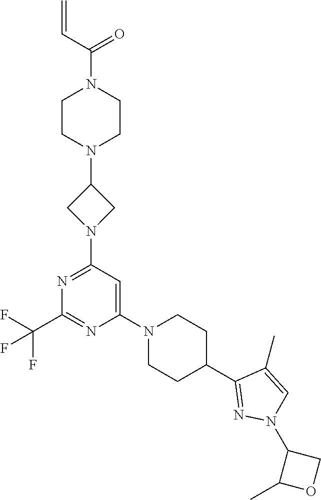 [SMILES: C=CC(=O)N1CCN(C2CN(c3cc(N4CCC(c5nn(C6COC6C)cc5C)CC4)nc(C(F)(F)F)n3)C2)CC1] | 1-(4-(1-(6-(4-(4-methyl- 1-(2-methyl-3- oxetanyl)-1H-pyrazol-3- yl)-1-piperidinyl)-2- (trifluoromethyl)-4- pyrimidinyl)-3- azetidinyl)-1- piperazinyl)-2-propen-1- one | E-3 | 574.7 |
| 2-156 | 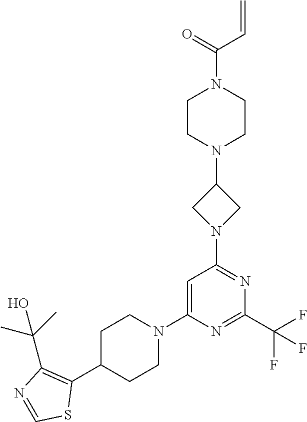 [SMILES: C=CC(=O)N1CCN(C2CN(c3cc(N4CCC(c5scnc5C(C)(C)O)CC4)nc(C(F)(F)F)n3)C2)CC1] | 1-(4-(1-(6-(4-(4-(2- hydroxy-2-propanyl)- 1,3-thiazol-5-yl)-1- piperidinyl)-2- (trifluoromethyl)-4- pyrimidinyl)-3- azetidinyl)-1- piperazinyl)-2-propen-1- one | E-3 | 566.7 |
| 2-157 | 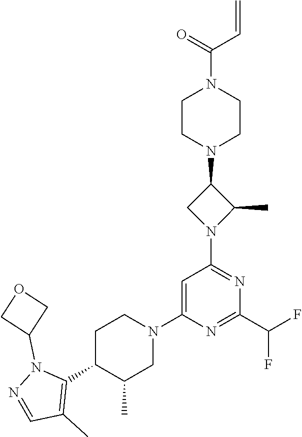 [SMILES: C=CC(=O)N1CCN([C@@H]2CN(c3cc(N4CC[C@@H](c5c(C)cnn5C5COC5)[C@@H](C)C4)nc(C(F)F)n3)[C@@H]2C)CC1] | 1-(4-((2R,3R)-1-(2- (difluoromethyl)-6- ((3R,4R)-3-methyl-4-(4- methyl-1-(3-oxetanyl)- 1H-pyrazol-5-yl)-1- piperidinyl)-4- pyrimidinyl)-2-methyl- 3-azetidinyl)-1- piperazinyl)-2-propen-1- one | E-2 | 571.2 |
| 2-158 | 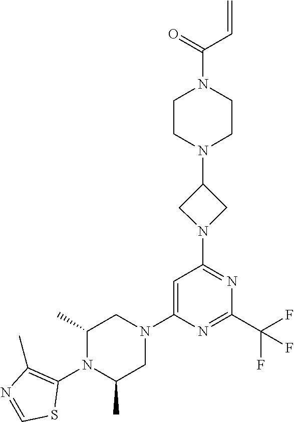 [SMILES: C=CC(=O)N1CCN(C2CN(c3cc(N4C[C@@H](C)N(c5scnc5C)[C@H](C)C4)nc(C(F)(F)F)n3)C2)CC1] | 1-(4-(1-(6-((3R,5R)-3,5- dimethyl-4-(4-methyl- 1,3-thiazol-5-yl)-1- piperazinyl)-2- (trifluoromethyl)-4- pyrimidinyl)-3- azetidinyl)-1- piperazinyl)-2-propen-1- one | E-3 | 551.2 |
| 2-159 | 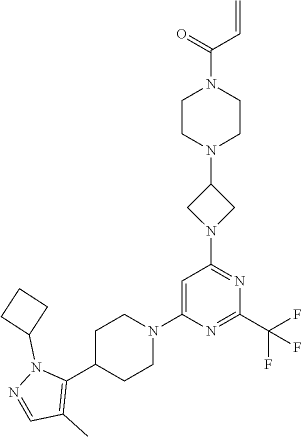 [SMILES: C=CC(=O)N1CCN(C2CN(c3cc(N4CCC(c5c(C)cnn5C5CCC5)CC4)nc(C(F)(F)F)n3)C2)CC1] | 1-(4-(1-(6-(4-(1- cyclobutyl-4-methyl- 1H-pyrazol-5-yl)-1- piperidinyl)-2- (trifluoromethyl)-4- pyrimidinyl)-3- azetidinyl)-1- piperazinyl)-2-propen-1- one | E-3 | 558.8 |
| 2-160 | 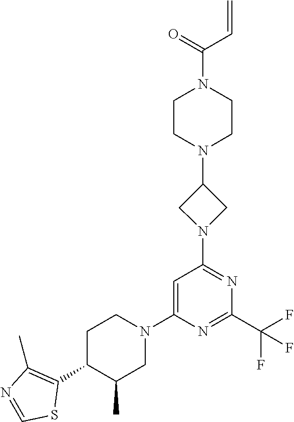 [SMILES: C=CC(=O)N1CCN(C2CN(c3cc(N4CC[C@@H](c5scnc5C)[C@H](C)C4)nc(C(F)(F)F)n3)C2)CC1] | 1-(4-(1-(6-((3S,4R)-3- methyl-4-(4-methyl-1,3- thiazol-5-yl)-1- piperidinyl)-2- (trifluoromethyl)-4- pyrimidinyl)-3- azetidinyl)-1- piperazinyl)-2-propen-1- one | E-3 | 536.7 |
| 2-161 | 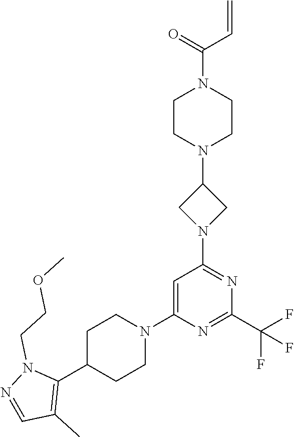 [SMILES: C=CC(=O)N1CCN(C2CN(c3cc(N4CCC(c5c(C)cnn5CCOC)CC4)nc(C(F)(F)F)n3)C2)CC1] | 1-(4-(1-(6-(4-(1-(2- methoxyethyl)-4- methyl-1H-pyrazol-5- yl)-1-piperidinyl)-2- (trifluoromethyl)-4- pyrimidinyl)-3- azetidinyl)-1- piperazinyl)-2-propen-1- one | E-3 | 562.8 |
| 2-162 | 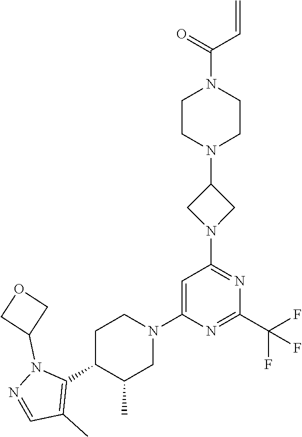 [SMILES: C=CC(=O)N1CCN(C2CN(c3cc(N4CC[C@@H](c5c(C)cnn5C5COC5)[C@@H](C)C4)nc(C(F)(F)F)n3)C2)CC1] | 1-(4-(1-(6-((3R,4R)-3- methyl-4-(4-methyl-1- (3-oxetanyl)-1H- pyrazol-5-yl)-1- piperidinyl)-2- (trifluoromethyl)-4- pyrimidinyl)-3- azetidinyl)-1- piperazinyl)-2-propen-1- one | E-3 | 574.7 |
| 2-163 | 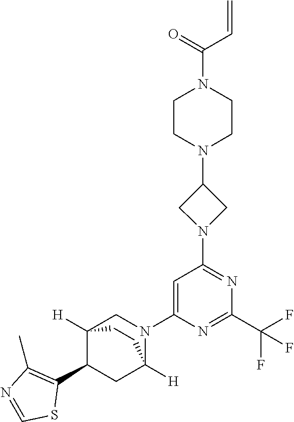 [SMILES: C=CC(=O)N1CCN(C2CN(c3cc(N4C[C@H]5CC[C@@H]4C[C@H]5c4scnc4C)nc(C(F)(F)F)n3)C2)CC1] | 1-(4-(1-(6-((1R,4S,5S)- 5-(4-methyl-1,3-thiazol- 5-yl)-2- azabicyclo[2.2.2]octan- 2-yl)-2- (trifluoromethyl)-4- pyrimidinyl)-3- azetidinyl)-1- piperazinyl)-2-propen-1- one | E-3 | 548.2 |
| 2-164 | 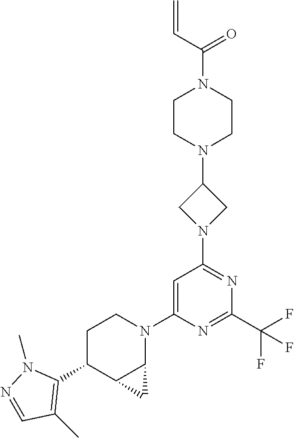 [SMILES: C=CC(=O)N1CCN(C2CN(c3cc(N4CC[C@@H](c5c(C)cnn5C)[C@H]5C[C@H]54)nc(C(F)(F)F)n3)C2)CC1] | 1-(4-(1-(6-((1R,5R,6R)- 5-(1,4-dimethyl-1H- pyrazol-5-yl)-2- azabicyclo[4.1.0]heptan- 2-yl)-2- (trifluoromethyl)-4- pyrimidinyl)-3- azetidinyl)-1- piperazinyl)-2-propen-1- one | E-3 | 530.7 |
| 2-165 | 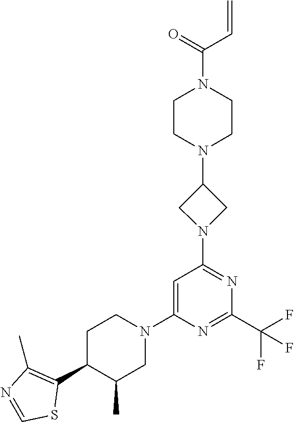 [SMILES: C=CC(=O)N1CCN(C2CN(c3cc(N4CC[C@H](c5scnc5C)[C@H](C)C4)nc(C(F)(F)F)n3)C2)CC1] | 1-(4-(1-(6-((3S,4S)-3- methyl-4-(4-methyl-1,3- thiazol-5-yl)-1- piperidinyl)-2- (trifluoromethyl)-4- pyrimidinyl)-3- azetidinyl)-1- piperazinyl)-2-propen-1- one | E-3 | 535.9 |
| 2-167 | 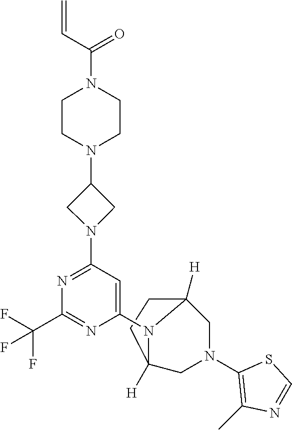 [SMILES: C=CC(=O)N1CCN(C2CN(c3cc(N4C5CCC4CN(c4scnc4C)C5)nc(C(F)(F)F)n3)C2)CC1] | 1-(4-(1-(6-(3-(4-methyl- 1,3-thiazol-5-yl)-3,8- diazabicyclo[3.2.1]octan- 8-yl)-2- (trifluoromethyl)-4- pyrimidinyl)-3- azetidinyl)-1- piperazinyl)-2-propen-1- one | E-3 | 548.8 |
| 2-168 | 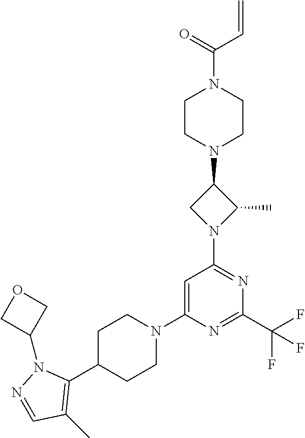 [SMILES: C=CC(=O)N1CCN([C@@H]2CN(c3cc(N4CCC(c5c(C)cnn5C5COC5)CC4)nc(C(F)(F)F)n3)[C@H]2C)CC1] | 1-(4-((2S,3R)-2-methyl- 1-(6-(4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-20 | 574.7 |
| 2-169 | 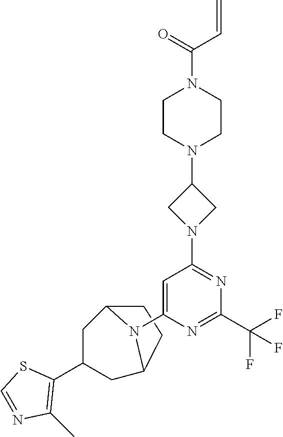 [SMILES: C=CC(=O)N1CCN(C2CN(c3cc(N4C5CCC4CC(c4scnc4C)C5)nc(C(F)(F)F)n3)C2)CC1] | 1-(4-(1-(6-(3-(4-methyl- 1,3-thiazol-5-yl)-8- azabicyclo[3.2.1]octan- 8-yl)-2- (trifluoromethyl)-4- pyrimidinyl)-3- azetidinyl)-1- piperazinyl)-2-propen-1- one | E-3 | 547.2 |
| 2-170 | 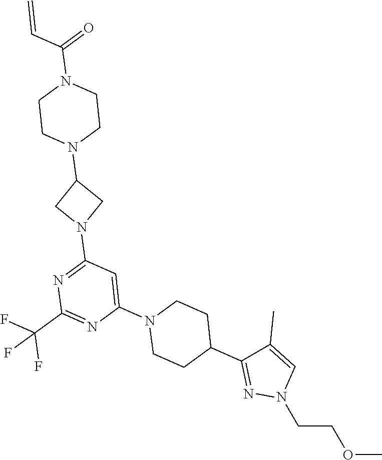 [SMILES: C=CC(=O)N1CCN(C2CN(c3cc(N4CCC(c5nn(CCOC)cc5C)CC4)nc(C(F)(F)F)n3)C2)CC1] | 1-(4-(1-(6-(4-(1-(2- methoxyethyl)-4- methyl-1H-pyrazol-3- yl)-1-piperidinyl)-2- (trifluoromethyl)-4- pyrimidinyl)-3- azetidinyl)-1- piperazinyl)-2-propen-1- one | E-3 | 562.8 |
| 2-171 | 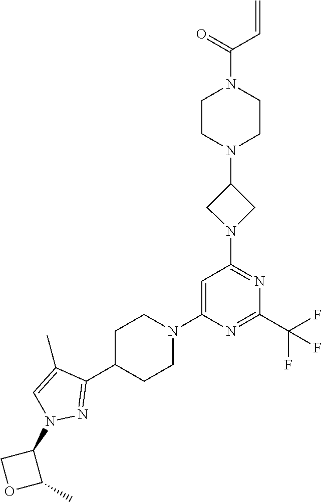 [SMILES: C=CC(=O)N1CCN(C2CN(c3cc(N4CCC(c5nn([C@@H]6CO[C@H]6C)cc5C)CC4)nc(C(F)(F)F)n3)C2)CC1] | 1-(4-(1-(6-(4-(4-methyl- 1-((2S,3R)-2-methyl-3- oxetanyl)-1H-pyrazol-3- yl)-1-piperidinyl)-2- (trifluoromethyl)-4- pyrimidinyl)-3- azetidinyl)-1- piperazinyl)-2-propen-1- one | E-3 | 574.7 |
| 2-173 | 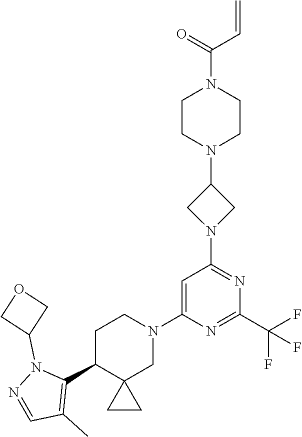 [SMILES: C=CC(=O)N1CCN(C2CN(c3cc(N4CC[C@H](c5c(C)cnn5C5COC5)C5(CC5)C4)nc(C(F)(F)F)n3)C2)CC1] | 1-(4-(1-(6-((8S)-8-(4- methyl-1-(3-oxetanyl)- 1H-pyrazol-5-yl)-5- azaspiro[2.5]octan-5- yl)-2-(trifluoromethyl)- 4-pyrimidinyl)-3- azetidinyl)-1- piperazinyl)-2-propen-1- one | E-3 | 586.7 |
| 2-175 | 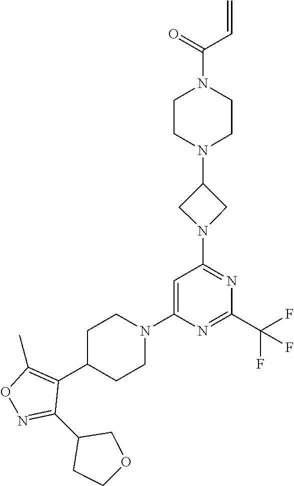 [SMILES: C=CC(=O)N1CCN(C2CN(c3cc(N4CCC(c5c(C6CCOC6)noc5C)CC4)nc(C(F)(F)F)n3)C2)CC1] | 1-(4-(1-(6-(4-(5-methyl- 3-(tetrahydro-3-furanyl)- 1,2-oxazol-4-yl)-1- piperidinyl)-2- (trifluoromethyl)-4- pyrimidinyl)-3- azetidinyl)-1- piperazinyl)-2-propen-1- one | E-3 | 576.3 |
| 2-176 | 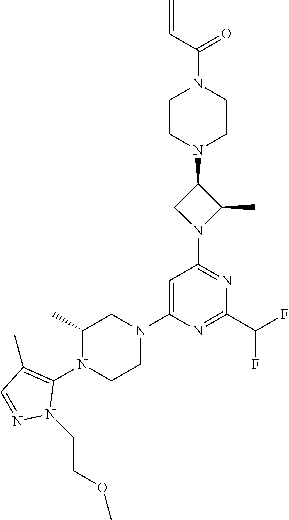 [SMILES: C=CC(=O)N1CCN([C@@H]2CN(c3cc(N4CCN(c5c(C)cnn5CCOC)[C@H](C)C4)nc(C(F)F)n3)[C@@H]2C)CC1] | 1-(4-((2R,3R)-1-(2- (difluoromethyl)-6- ((3R)-4-(1-(2- methoxyethyl)-4- methyl-1H-pyrazol-5- yl)-3-methyl-1- piperazinyl)-4- pyrimidinyl)-2-methyl- 3-azetidinyl)-1- piperazinyl)-2-propen-1- one | E-8 | 574.0 |
| 2-177 | 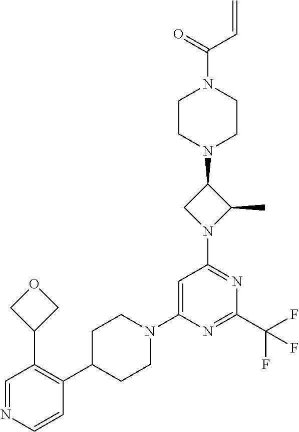 [SMILES: C=CC(=O)N1CCN([C@@H]2CN(c3cc(N4CCC(c5ccncc5C5COC5)CC4)nc(C(F)(F)F)n3)[C@@H]2C)CC1] | 1-(4-((2R,3R)-2-methyl- 1-(6-(4-(3-(3-oxetanyl)- 4-pyridinyl)-1- piperidinyl)-2- (trifluoromethyl)-4- pyrimidinyl)-3- azetidinyl)-1- piperazinyl)-2-propen-1- one | E-1 | 572.2 |
| 2-178 | 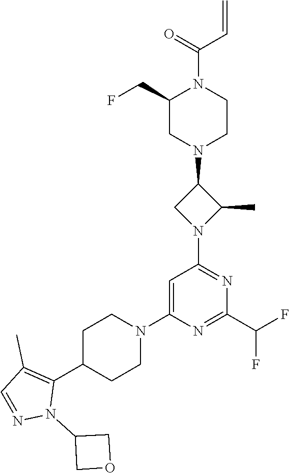 [SMILES: C=CC(=O)N1CCN([C@@H]2CN(c3cc(N4CCC(c5c(C)cnn5C5COC5)CC4)nc(C(F)F)n3)[C@@H]2C)C[C@H]1CF] | 1-((2S)-4-((2R,3R)-1-(2- (difluoromethyl)-6-(4- (4-methyl-1-(3- oxetanyl)-1H-pyrazol-5- yl)-1-piperidinyl)-4- pyrimidinyl)-2-methyl- 3-azetidinyl)-2- (fluoromethyl)-1- piperazinyl)-2-propen-1- one | E-19 | 589.3 |
| 2-179 | 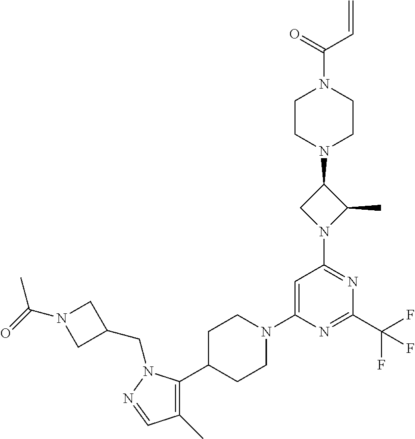 [SMILES: C=CC(=O)N1CCN([C@@H]2CN(c3cc(N4CCC(c5c(C)cnn5CC5CN(C(C)=O)C5)CC4)nc(C(F)(F)F)n3)[C@@H]2C)CC1] | 1-(4-((2R,3R)-1-(6-(4- (1-((1-acetyl-3- azetidinyl)methyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-8 | 630.3 |
| 2-180 | 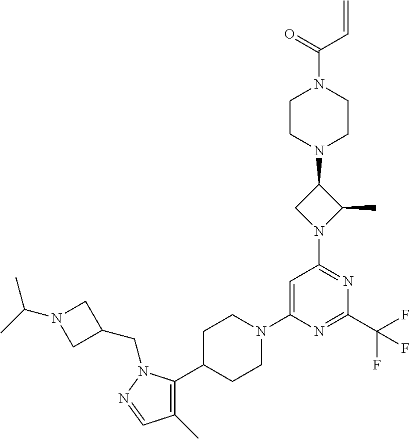 [SMILES: C=CC(=O)N1CCN([C@@H]2CN(c3cc(N4CCC(c5c(C)cnn5CC5CN(C(C)C)C5)CC4)nc(C(F)(F)F)n3)[C@@H]2C)CC1] | 1-(4-((2R,3R)-2-methyl- 1-(6-(4-(4-methyl-1-((1- (2-propanyl)-3- azetidinyl)methyl)-1H- pyrazol-5-yl)-1- piperidinyl)-2- (trifluoromethyl)-4- pyrimidinyl)-3- azetidinyl)-1- piperazinyl)-2-propen-1- one | E-8 | 630.3 |
| 2-181 | 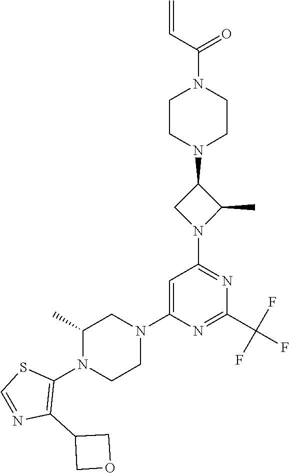 [SMILES: C=CC(=O)N1CCN([C@@H]2CN(c3cc(N4CCN(c5scnc5C5COC5)[C@H](C)C4)nc(C(F)(F)F)n3)[C@@H]2C)CC1] | 1-(4-((2R,3R)-2-methyl- 1-(6-((3R)-3-methyl-4- (4-(3-oxetanyl)-1,3- thiazol-5-yl)-1- piperazinyl)-2- (trifluoromethyl)-4- pyrimidinyl)-3- azetidinyl)-1- piperazinyl)-2-propen-1- one | E-8 | 592.9 |
| 2-182 | 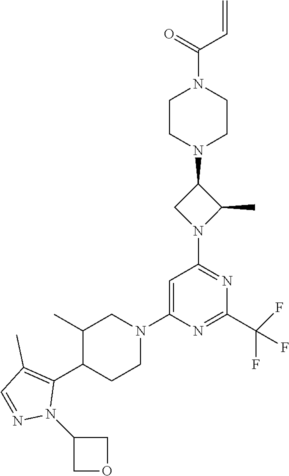 [SMILES: C=CC(=O)N1CCN([C@@H]2CN(c3cc(N4CCC(c5c(C)cnn5C5COC5)C(C)C4)nc(C(F)(F)F)n3)[C@@H]2C)CC1] | 1-(4-((2R,3R)-2-methyl- 1-(6-((3R,4R)-3-methyl- 4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-8 | 589.4 |
| 2-184 | 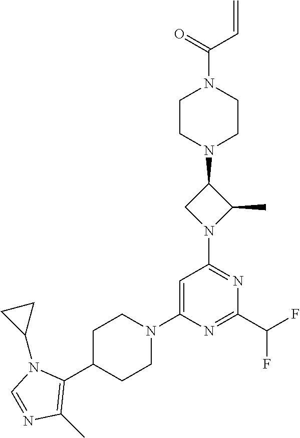 [SMILES: C=CC(=O)N1CCN([C@@H]2CN(c3cc(N4CCC(c5c(C)ncn5C5CC5)CC4)nc(C(F)F)n3)[C@@H]2C)CC1] | 1-(4-((2R,3R)-1-(6-(4- (1-cyclopropyl-4- methyl-1H-imidazol-5- yl)-1-piperidinyl)-2- (difluoromethyl)-4- pyrimidinyl)-2-methyl- 3-azetidinyl)-1- piperazinyl)-2-propen-1- one | E-9 | 541.3 |
| 2-185 | 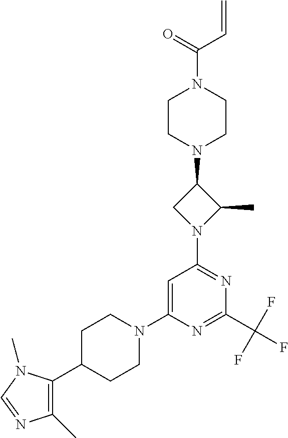 [SMILES: C=CC(=O)N1CCN([C@@H]2CN(c3cc(N4CCC(c5c(C)ncn5C)CC4)nc(C(F)(F)F)n3)[C@@H]2C)CC1] | 1-(4-((2R,3R)-1-(6-(4- (1,4-dimethyl-1H- imidazol-5-yl)-1- piperidinyl)-2- (trifluoromethyl)-4- pyrimidinyl)-2-methyl- 3-azetidinyl)-1- piperazinyl)-2-propen-1- one | E-8 | 533.4 |
| 2-186 | 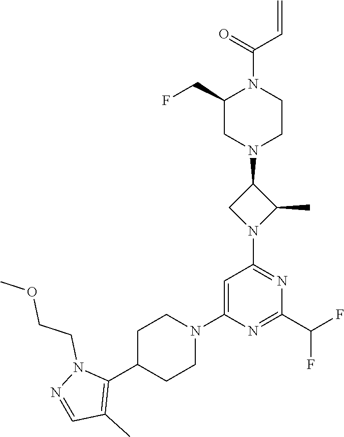 [SMILES: C=CC(=O)N1CCN([C@@H]2CN(c3cc(N4CCC(c5c(C)cnn5CCOC)CC4)nc(C(F)F)n3)[C@@H]2C)C[C@H]1CF] | 1-((2S)-4-((2R,3R)-1-(2- (difluoromethyl)-6-(4- (1-(2-methoxyethyl)-4- methyl-1H-pyrazol-5- yl)-1-piperidinyl)-4- pyrimidinyl)-2-methyl- 3-azetidinyl)-2- (fluoromethyl)-1- piperazinyl)-2-propen-1- one | | 591.3 |
| 2-187 | 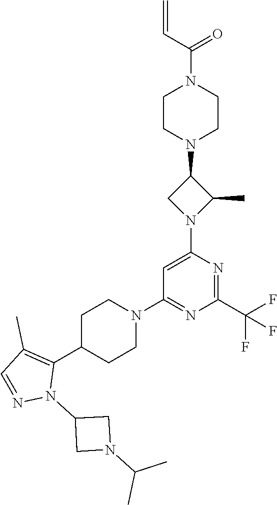 [SMILES: C=CC(=O)N1CCN([C@@H]2CN(c3cc(N4CCC(c5c(C)cnn5C5CN(C(C)C)C5)CC4)nc(C(F)(F)F)n3)[C@@H]2C)CC1] | 1-(4-((2R,3R)-2-methyl- 1-(6-(4-(4-methyl-1-(1- (2-propanyl)-3- azetidinyl)-1H-pyrazol- 5-yl)-1-piperidinyl)-2- (trifluoromethyl)-4- pyrimidinyl)-3- azetidinyl)-1- piperazinyl)-2-propen-1- one | E-8 | 616.2 |
| 2-188 | 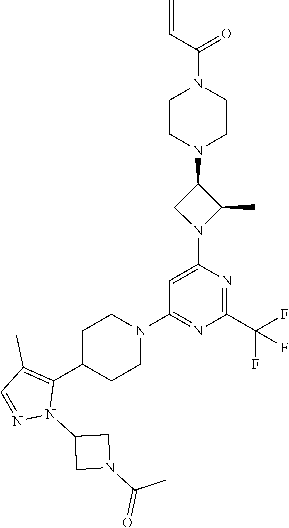 [SMILES: C=CC(=O)N1CCN([C@@H]2CN(c3cc(N4CCC(c5c(C)cnn5C5CN(C(C)=O)C5)CC4)nc(C(F)(F)F)n3)[C@@H]2C)CC1] | 1-(4-((2R,3R)-1-(6-(4- (1-(1-acetyl-3- azetidinyl)-4-methyl- 1H-pyrazol-5-yl)-1- piperidinyl)-2- (trifluoromethyl)-4- pyrimidinyl)-2-methyl- 3-azetidinyl)-1- piperazinyl)-2-propen-1- one | E-8 | 616.2 |
| 2-189 | 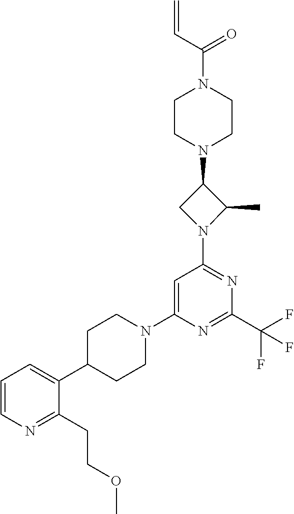 [SMILES: C=CC(=O)N1CCN([C@@H]2CN(c3cc(N4CCC(c5cccnc5CCOC)CC4)nc(C(F)(F)F)n3)[C@@H]2C)CC1] | 1-(4-((2R,3R)-1-(6-(4- (2-(2-methoxyethyl)-3- pyridinyl)-1- piperidinyl)-2- (trifluoromethyl)-4- pyrimidinyl)-2-methyl- 3-azetidinyl)-1- piperazinyl)-2-propen-1- one | E-8 | 574.2 |
| 2-190 | 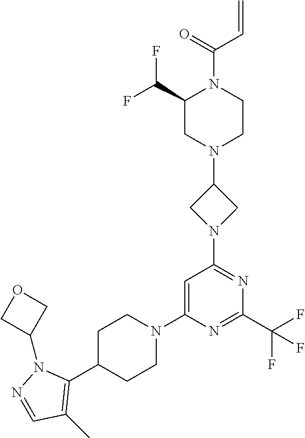 [SMILES: C=CC(=O)N1CCN(C2CN(c3cc(N4CCC(c5c(C)cnn5C5COC5)CC4)nc(C(F)(F)F)n3)C2)C[C@H]1C(F)F] | 1-((2S)-2- (difluoromethyl)-4-(1- (6-(4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-7 | 611.3 |
| 2-191 | 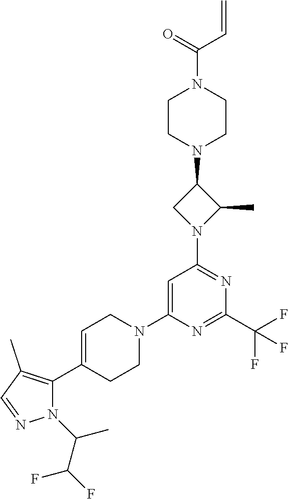 [SMILES: C=CC(=O)N1CCN([C@@H]2CN(c3cc(N4CC=C(c5c(C)cnn5C(C)C(F)F)CC4)nc(C(F)(F)F)n3)[C@@H]2C)CC1] | 1-(4-((2R,3R)-1-(6-(4- (1-(1,1-difluoro-2- propanyl)-4-methyl-1H- pyrazol-5-yl)-3,6- dihydro-1(2H)- pyridinyl)-2- (trifluoromethyl)-4- pyrimidinyl)-2-methyl- 3-azetidinyl)-1- piperazinyl)-2-propen-1- one | E-8 | 595.3 |
| 2-192 | 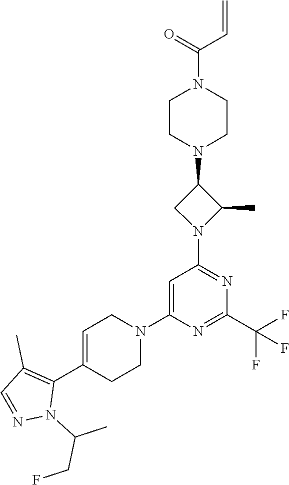 [SMILES: C=CC(=O)N1CCN([C@@H]2CN(c3cc(N4CC=C(c5c(C)cnn5C(C)CF)CC4)nc(C(F)(F)F)n3)[C@@H]2C)CC1] | 1-(4-((2R,3R)-1-(6-(4- (1-(1-fluoro-2- propanyl)-4-methyl-1H- pyrazol-5-yl)-3,6- dihydro-1(2H)- pyridinyl)-2- (trifluoromethyl)-4- pyrimidinyl)-2-methyl- 3-azetidinyl)-1- piperazinyl)-2-propen-1- one | E-8 | 577.3 |
| 2-193 | 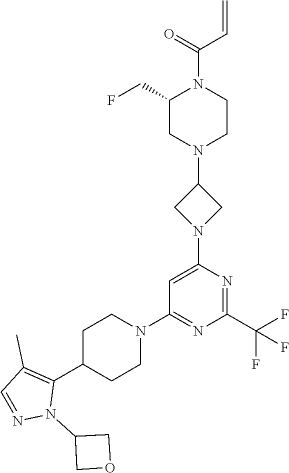 [SMILES: C=CC(=O)N1CCN(C2CN(c3cc(N4CCC(c5c(C)cnn5C5COC5)CC4)nc(C(F)(F)F)n3)C2)C[C@@H]1CF] | 1-((2R)-2- (fluoromethyl)-4-(1-(6- (4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-16 | 593.3 |
| 2-194 | 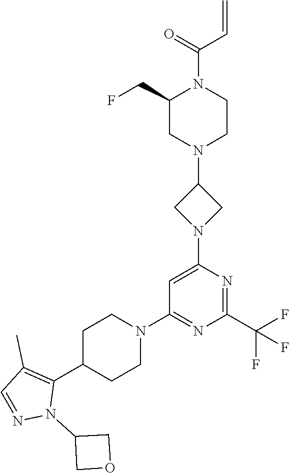 [SMILES: C=CC(=O)N1CCN(C2CN(c3cc(N4CCC(c5c(C)cnn5C5COC5)CC4)nc(C(F)(F)F)n3)C2)C[C@H]1CF] | 1-((2S)-2- (fluoromethyl)-4-(1-(6- (4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-16 | 593.3 |
| 2-195 | 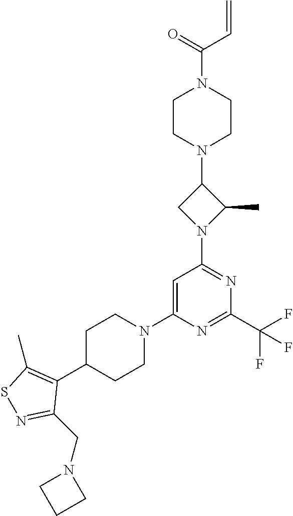 [SMILES: C=CC(=O)N1CCN(C2CN(c3cc(N4CCC(c5c(CN6CCC6)nsc5C)CC4)nc(C(F)(F)F)n3)[C@@H]2C)CC1] | 1-(4-((2R,3R)-1-(6-(4- (3-(1-azetidinylmethyl)- 5-methyl-1,2-thiazol-4- yl)-1-piperidinyl)-2- (trifluoromethyl)-4- pyrimidinyl)-2-methyl- 3-azetidinyl)-1- piperazinyl)-2-propen-1- one | E-8 | 605.2 |
| 2-196 | 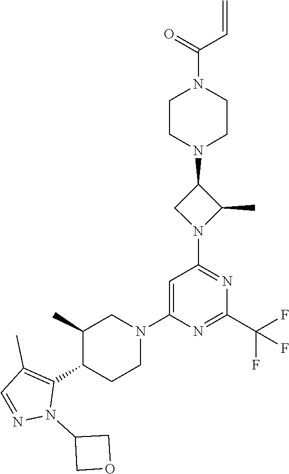 [SMILES: C=CC(=O)N1CCN([C@@H]2CN(c3cc(N4CC[C@H](c5c(C)cnn5C5COC5)[C@@H](C)C4)nc(C(F)(F)F)n3)[C@@H]2C)CC1] | 1-(4-((2R,3R)-2-methyl- 1-(6-((3R,4S)-3-methyl- 4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-8 | 589.4 |
| | mixture trans isomers at piperidine | | | |
| 2-197 | 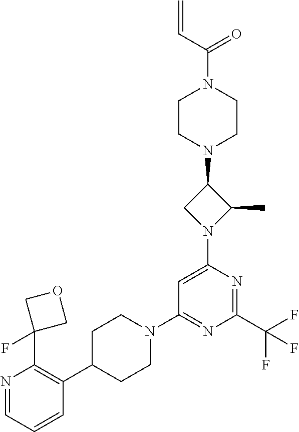 [SMILES: C=CC(=O)N1CCN([C@@H]2CN(c3cc(N4CCC(c5cccnc5C5(F)COC5)CC4)nc(C(F)(F)F)n3)[C@@H]2C)CC1] | 1-(4-((2R,3R)-1-(6-(4- (2-(3-fluoro-3- oxetanyl)-3-pyridinyl)- 1-piperidinyl)-2- (trifluoromethyl)-4- pyrimidinyl)-2-methyl- 3-azetidinyl)-1- piperazinyl)-2-propen-1- one | E-8 | 590.3 |
| 2-198 | 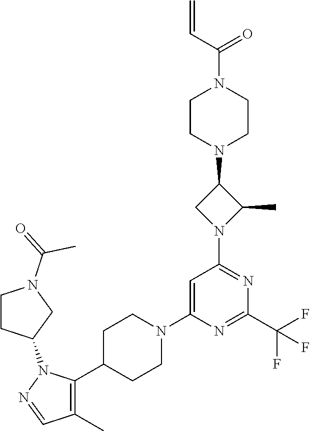 [SMILES: C=CC(=O)N1CCN([C@@H]2CN(c3cc(N4CCC(c5c(C)cnn5[C@@H]5CCN(C(C)=O)C5)CC4)nc(C(F)(F)F)n3)[C@@H]2C)CC1] | 1-(4-((2R,3R)-1-(6-(4- (1-((3R)-1-acetyl-3- pyrrolidinyl)-4-methyl- 1H-pyrazol-5-yl)-1- piperidinyl)-2- (trifluoromethyl)-4- pyrimidinyl)-2-methyl- 3-azetidinyl)-1- piperazinyl)-2-propen-1- one | E-8 | 630.2 |
| 2-199 | 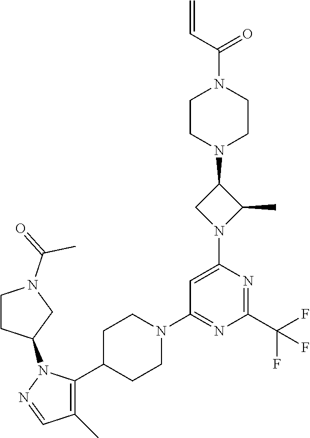 [SMILES: C=CC(=O)N1CCN([C@@H]2CN(c3cc(N4CCC(c5c(C)cnn5[C@H]5CCN(C(C)=O)C5)CC4)nc(C(F)(F)F)n3)[C@@H]2C)CC1] | 1-(4-((2R,3R)-1-(6-(4- (1-((3S)-1-acetyl-3- pyrrolidinyl)-4-methyl- 1H-pyrazol-5-yl)-1- piperidinyl)-2- (trifluoromethyl)-4- pyrimidinyl)-2-methyl- 3-azetidinyl)-1- piperazinyl)-2-propen-1- one | E-8 | 630.3 |
| 2-200 | 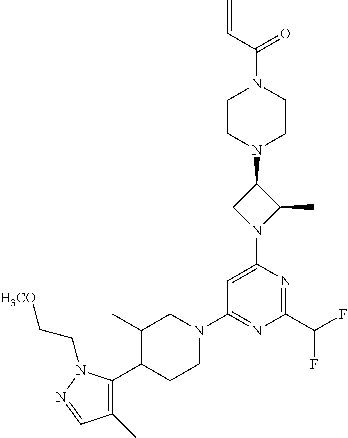 [SMILES: C=CC(=O)N1CCN([C@@H]2CN(c3cc(N4CCC(c5c(C)cnn5CCOC)C(C)C4)nc(C(F)F)n3)[C@@H]2C)CC1] | 1-(4-((2R,3R)-1-(2- (difluoromethyl)-6-(4- (1-(2-methoxyethyl)-4- methyl-1H-pyrazol-5- yl)-3-methyl-1- piperidinyl)-4- pyrimidinyl)-2-methyl- 3-azetidinyl)-1- piperazinyl)-2-propen-1- one | E-9 | 573.2 |
| 2-201 | 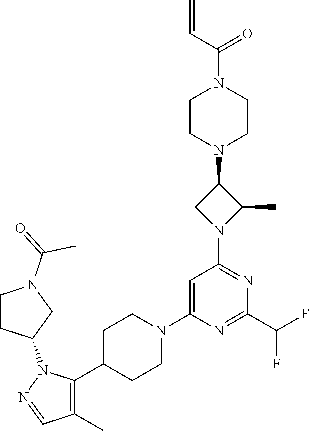 [SMILES: C=CC(=O)N1CCN([C@@H]2CN(c3cc(N4CCC(c5c(C)cnn5[C@@H]5CCN(C(C)=O)C5)CC4)nc(C(F)F)n3)[C@@H]2C)CC1] | 1-(4-((2R,3R)-1-(6-(4- (1-((3R)-1-acetyl-3- pyrrolidinyl)-4-methyl- 1H-pyrazol-5-yl)-1- piperidinyl)-2- (difluoromethyl)-4- pyrimidinyl)-2-methyl- 3-azetidinyl)-1- piperazinyl)-2-propen-1- one | E-9 | 612.3 |
| 2-202 | 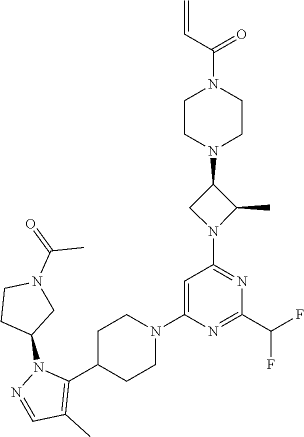 [SMILES: C=CC(=O)N1CCN([C@@H]2CN(c3cc(N4CCC(c5c(C)cnn5[C@H]5CCN(C(C)=O)C5)CC4)nc(C(F)F)n3)[C@@H]2C)CC1] | 1-(4-((2R,3R)-1-(6-(4- (1-((3S)-1-acetyl-3- pyrrolidinyl)-4-methyl- 1H-pyrazol-5-yl)-1- piperidinyl)-2- (difluoromethyl)-4- pyrimidinyl)-2-methyl- 3-azetidinyl)-1- piperazinyl)-2-propen-1- one | E-9 | 612.2 |
| 2-203 | 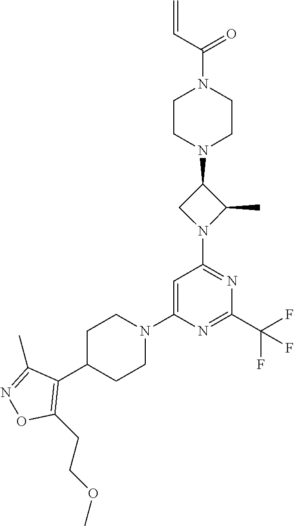 [SMILES: C=CC(=O)N1CCN([C@@H]2CN(c3cc(N4CCC(c5c(C)noc5CCOC)CC4)nc(C(F)(F)F)n3)[C@@H]2C)CC1] | 1-(4-((2R,3R)-1-(6-(4- (5-(2-methoxyethyl)-3- methyl-1.2-oxazol-4-yl)- 1-piperidinyl)-2- (trifluoromethyl)-4- pyrimidinyl)-2-methyl- 3-azetidinyl)-1- piperazinyl)-2-propen-1- one | E-8 | 578.3 |
| 2-204 | 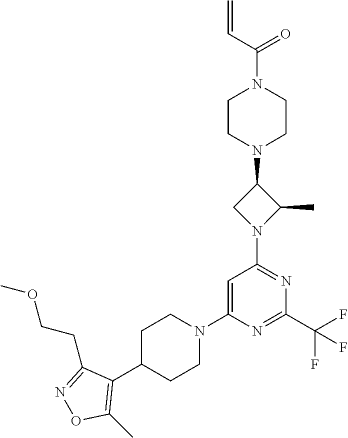 [SMILES: C=CC(=O)N1CCN([C@@H]2CN(c3cc(N4CCC(c5c(CCOC)noc5C)CC4)nc(C(F)(F)F)n3)[C@@H]2C)CC1] | 1-(4-((2R,3R)-1-(6-(4- (3-(2-methoxyethyl)-5- methyl-1,2-oxazol-4-yl)- 1-piperidinyl)-2- (trifluoromethyl)-4- pyrimidinyl)-2-methyl- 3-azetidinyl)-1- piperazinyl)-2-propen-1- one | E-8 | 578.3 |
| 2-205 | 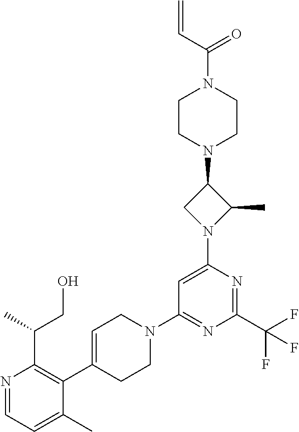 [SMILES: C=CC(=O)N1CCN([C@@H]2CN(c3cc(N4CC=C(c5c(C)ccnc5[C@H](C)CO)CC4)nc(C(F)(F)F)n3)[C@@H]2C)CC1] | 1-(4-((2R,3R)-1-(6-(2- ((2S)-1-hydroxy-2- propanyl)-4-methyl- 3′,6′-dihydro[3,4′- bipyridin]-1′(2′H)-yl)-2- (trifluoromethyl)-4- pyrimidinyl)-2-methyl- 3-azetidinyl)-1- piperazinyl)-2-propen-1- one | E-1 | 586.3 |
| 2-206 | 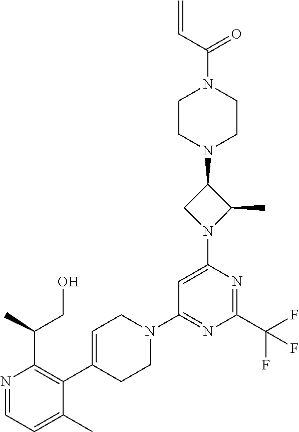 [SMILES: C=CC(=O)N1CCN([C@@H]2CN(c3cc(N4CC=C(c5c(C)ccnc5[C@@H](C)CO)CC4)nc(C(F)(F)F)n3)[C@@H]2C)CC1] | 1-(4-((2R,3R)-1-(6-(2- ((2R)-1-hydroxy-2- propanyl)-4-methyl- 3′,6′-dihydro[3,4′- bipyridin]-1′(2′H)-yl)-2- (trifluoromethyl)-4- pyrimidinyl)-2-methyl- 3-azetidinyl)-1- piperazinyl)-2-propen-1- one | E-1 | 586.3 |
| 2-207 | 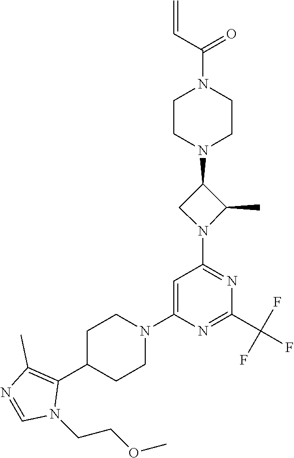 [SMILES: C=CC(=O)N1CCN([C@@H]2CN(c3cc(N4CCC(c5c(C)ncn5CCOC)CC4)nc(C(F)(F)F)n3)[C@@H]2C)CC1] | 1-(4-((2R,3R)-1-(6-(4- (1-(2-methoxyethyl)-4- methyl-1H-imidazol-5- yl)-1-piperidinyl)-2- (trifluoromethyl)-4- pyrimidinyl)-2-methyl- 3-azetidinyl)-1- piperazinyl)-2-propen-1- one | E-8 | 577.0 |
| 2-208 | 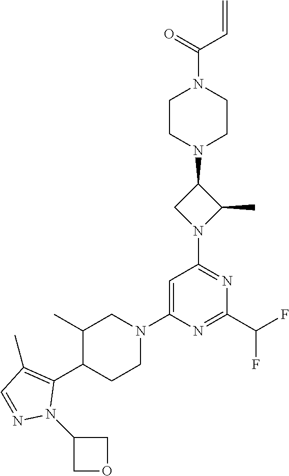 [SMILES: C=CC(=O)N1CCN([C@@H]2CN(c3cc(N4CCC(c5c(C)cnn5C5COC5)C(C)C4)nc(C(F)F)n3)[C@@H]2C)CC1] | 1-(4-((2R,3R)-1-(2- (difluoromethyl)-6-(-3- methyl-4-(4-methyl-1- (3-oxetanyl)-1H- pyrazol-5-yl)-1- piperidinyl)-4- pyrimidinyl)-2-methyl- 3-azetidinyl)-1- piperazinyl)-2-propen-1- one | E-9 | 571.2 |
| 2-209 | 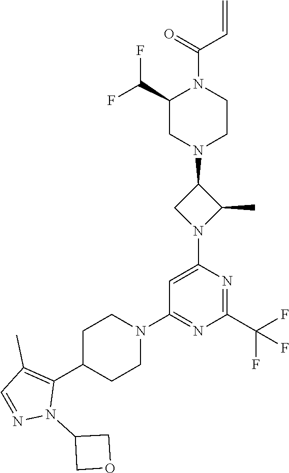 [SMILES: C=CC(=O)N1CCN([C@@H]2CN(c3cc(N4CCC(c5c(C)cnn5C5COC5)CC4)nc(C(F)(F)F)n3)[C@@H]2C)C[C@H]1C(F)F] | 1-((2S)-2- (difluoromethyl)-4- ((2R,3R)-2-methyl-1-(6- (4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-18 | 625.1 |
| 2-210 | 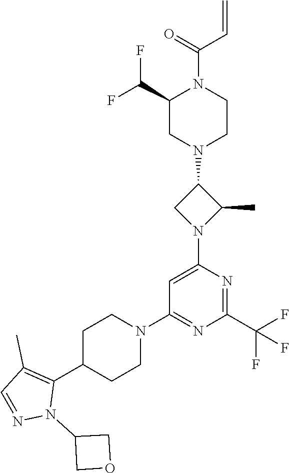 [SMILES: C=CC(=O)N1CCN([C@H]2CN(c3cc(N4CCC(c5c(C)cnn5C5COC5)CC4)nc(C(F)(F)F)n3)[C@@H]2C)C[C@H]1C(F)F] | 1-((2S)-2- (difluoromethyl)-4- ((2R,3S)-2-methyl-1-(6- (4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-7 | 625.3 |
| 2-211 | 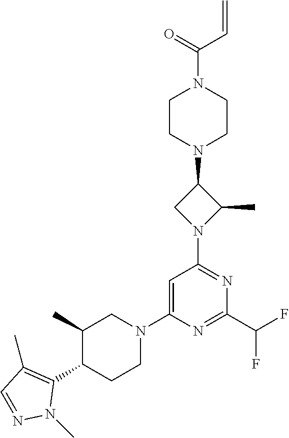 [SMILES: C=CC(=O)N1CCN([C@@H]2CN(c3cc(N4CC[C@H](c5c(C)cnn5C)[C@@H](C)C4)nc(C(F)F)n3)[C@@H]2C)CC1] | 1-(4-((2R,3R)-1-(2- (difluoromethyl)-6- ((3R,4S)-4-(1,4- dimethyl-1H-pyrazol-5- yl)-3-methyl-1- piperidinyl)-4- pyrimidinyl)-2-methyl- 3-azetidinyl)-1- piperazinyl)-2-propen-1- one | E-9 | 529.2 |
| 2-212 | 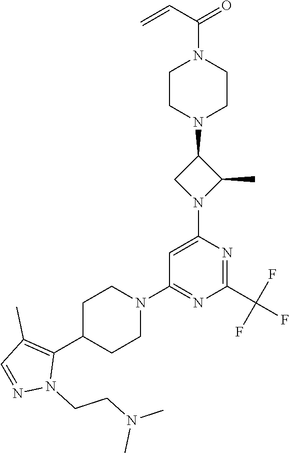 [SMILES: C=CC(=O)N1CCN([C@@H]2CN(c3cc(N4CCC(c5c(C)cnn5CCN(C)C)CC4)nc(C(F)(F)F)n3)[C@@H]2C)CC1] | 1-(4-((2R,3R)-1-(6-(4- (1-(2- (dimethylamino)ethyl)- 4-methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-8 | 590.2 |
| 2-213 | 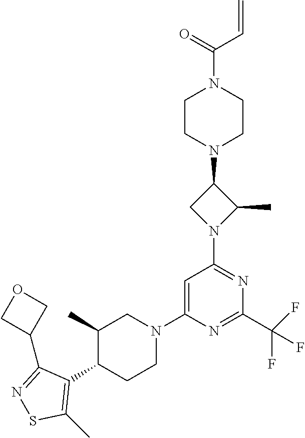 [SMILES: C=CC(=O)N1CCN([C@@H]2CN(c3cc(N4CC[C@H](c5c(C6COC6)nsc5C)[C@@H](C)C4)nc(C(F)(F)F)n3)[C@@H]2C)CC1] | 1-(4-((2R,3R)-2-methyl- 1-(6-((3R,4S)-3-methyl- 4-(5-methyl-3-(oxetan- 3-yl)isothiazol-4- yl)piperidin-1-yl)-2- (trifluoromethyl)pyrimidin- 4-yl)azetidin-3- yl)piperazin-1-yl)prop- 2-en-1-one | E-1 | 606.2 |
| | mixture of trans isomers at piperidine | | | |
| 2-214 | 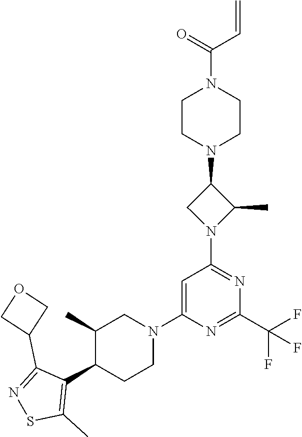 [SMILES: C=CC(=O)N1CCN([C@@H]2CN(c3cc(N4CC[C@@H](c5c(C6COC6)nsc5C)[C@@H](C)C4)nc(C(F)(F)F)n3)[C@@H]2C)CC1] | 1-(4-((2R,3R)-2-methyl- 1-(6-((3R,4R)-3-methyl- 4-(5-methyl-3-(3- oxetanyl)-1,2-thiazol-4- yl)-1-piperidinyl)-2- (trifluoromethyl)-4- pyrimidinyl)-3- azetidinyl)-1- piperazinyl)-2-propen-1- one | E-1 | 606.2 |
| 2-215 | 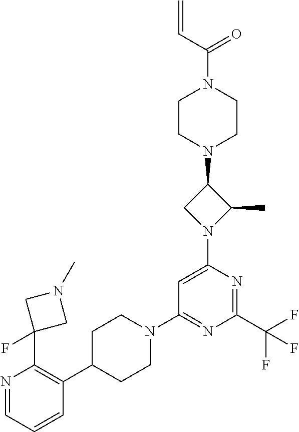 [SMILES: C=CC(=O)N1CCN([C@@H]2CN(c3cc(N4CCC(c5cccnc5C5(F)CN(C)C5)CC4)nc(C(F)(F)F)n3)[C@@H]2C)CC1] | 1-(4-((2R,3R)-1-(6-(4- (2-(3-fluoro-1-methyl-3- azetidinyl)-3-pyridinyl)- 1-piperidinyl)-2- (trifluoromethyl)-4- pyrimidinyl)-2-methyl- 3-azetidinyl)-1- piperazinyl)-2-propen-1- one | E-8 | 603.3 |
| 2-216 | 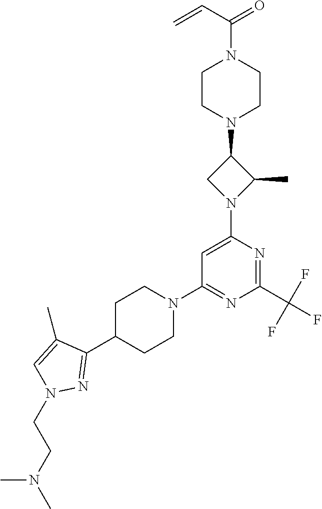 [SMILES: C=CC(=O)N1CCN([C@@H]2CN(c3cc(N4CCC(c5nn(CCN(C)C)cc5C)CC4)nc(C(F)(F)F)n3)[C@@H]2C)CC1] | 1-(4-((2R,3R)-1-(6-(4- (1-(2- (dimethylamino)ethyl)- 4-methyl-1H-pyrazol-3- yl)-1-piperidinyl)-2- (trifluoromethyl)-4- pyrimidinyl)-2-methyl- 3-azetidinyl)-1- piperazinyl)-2-propen-1- one | E-1 | 590.1 |
| 2-217 | 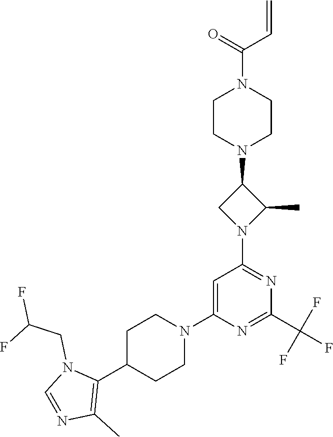 [SMILES: C=CC(=O)N1CCN([C@@H]2CN(c3cc(N4CCC(c5c(C)ncn5CC(F)F)CC4)nc(C(F)(F)F)n3)[C@@H]2C)CC1] | 1-(4-((2R,3R)-1-(6-(4- (1-(2,2-difluoroethyl)-4- methyl-1H-imidazol-5- yl)-1-piperidinyl)-2- (trifluoromethyl)-4- pyrimidinyl)-2-methyl- 3-azetidinyl)-1- piperazinyl)-2-propen-1- one | E-1 | 583.3 |
| 2-218 | 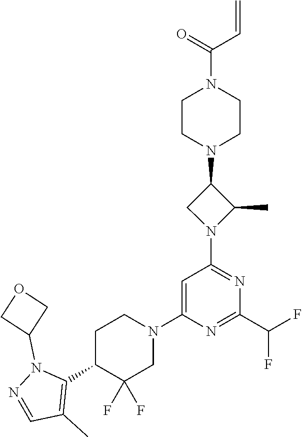 [SMILES: C=CC(=O)N1CCN([C@@H]2CN(c3cc(N4CC[C@@H](c5c(C)cnn5C5COC5)C(F)(F)C4)nc(C(F)F)n3)[C@@H]2C)CC1] | 1-(4-((2R,3R)-1-(2- (difluoromethyl)-6- ((4S)-3,3-difluoro-4-(4- methyl-1-(3-oxetanyl)- 1H-pyrazol-5-yl)-1- piperidinyl)-4- pyrimidinyl)-2-methyl- 3-azetidinyl)-1- piperazinyl)-2-propen-1- one | E-2 | 593.2 |
| 2-219 | 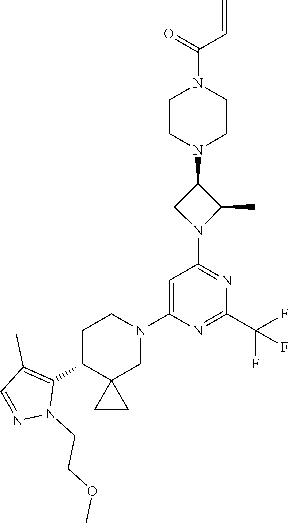 [SMILES: C=CC(=O)N1CCN([C@@H]2CN(c3cc(N4CC[C@@H](c5c(C)cnn5CCOC)C5(CC5)C4)nc(C(F)(F)F)n3)[C@@H]2C)CC1] | 1-(4-((2R,3R)-1-(6- ((8R)-8-(1-(2- methoxyethyl)-4- methyl-1H-pyrazol-5- yl)-5- azaspiro[2.5]octan-5- yl)-2-(trifluoromethyl)- 4-pyrimidinyl)-2- methyl-3-azetidinyl)-1- piperazinyl)-2-propen-1- one | E-1 | 603.2 |
| 2-220 | 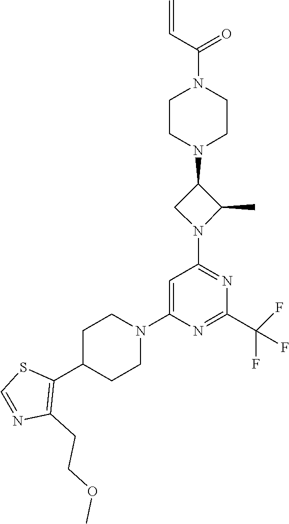 [SMILES: C=CC(=O)N1CCN([C@@H]2CN(c3cc(N4CCC(c5scnc5CCOC)CC4)nc(C(F)(F)F)n3)[C@@H]2C)CC1] | 1-(4-((2R,3R)-1-(6-(4- (4-(2-methoxyethyl)- 1,3-thiazol-5-yl)-1- piperidinyl)-2- (trifluoromethyl)-4- pyrimidinyl)-2-methyl- 3-azetidinyl)-1- piperazinyl)-2-propen-1- one | E-1 | 580.2 |
| 2-221 | 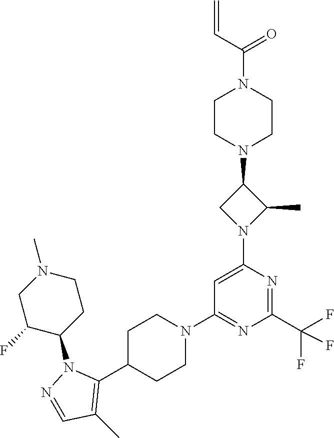 [SMILES: C=CC(=O)N1CCN([C@@H]2CN(c3cc(N4CCC(c5c(C)cnn5[C@@H]5CCN(C)C[C@H]5F)CC4)nc(C(F)(F)F)n3)[C@@H]2C)CC1] | 1-(4-((2R,3R)-1-(6-(4- (1-((3R,4R)-3-fluoro-1- methyl-4-piperidinyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 634.0 |
| 2-222 | 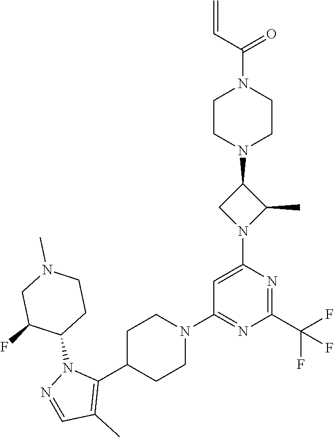 [SMILES: C=CC(=O)N1CCN([C@@H]2CN(c3cc(N4CCC(c5c(C)cnn5[C@H]5CCN(C)C[C@@H]5F)CC4)nc(C(F)(F)F)n3)[C@@H]2C)CC1] | 1-(4-((2R,3R)-1-(6-(4- (1-((3S,4S)-3-fluoro-1- methyl-4-piperidinyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 634.0 |
| 2-223 | 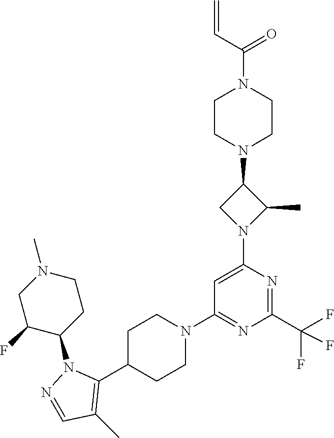 [SMILES: C=CC(=O)N1CCN([C@@H]2CN(c3cc(N4CCC(c5c(C)cnn5[C@@H]5CCN(C)C[C@@H]5F)CC4)nc(C(F)(F)F)n3)[C@@H]2C)CC1] | 1-(4-((2R,3R)-1-(6-(4- (1-((3S,4R)-3-fluoro-1- methyl-4-piperidinyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 634.0 |
| 2-224 | 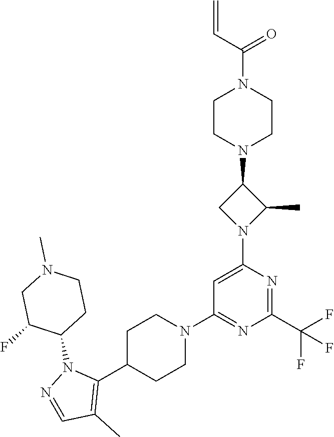 [SMILES: C=CC(=O)N1CCN([C@@H]2CN(c3cc(N4CCC(c5c(C)cnn5[C@H]5CCN(C)C[C@H]5F)CC4)nc(C(F)(F)F)n3)[C@@H]2C)CC1] | 1-(4-((2R,3R)-1-(6-(4- (1-((3R,4S)-3-fluoro-1- methyl-4-piperidinyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 634.0 |
| 2-225 | 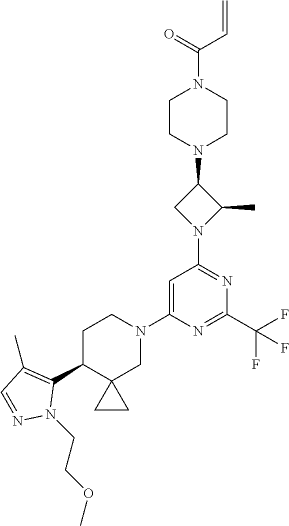 [SMILES: C=CC(=O)N1CCN([C@@H]2CN(c3cc(N4CC[C@H](c5c(C)cnn5CCOC)C5(CC5)C4)nc(C(F)(F)F)n3)[C@@H]2C)CC1] | 1-(4-((2R,3R)-1-(6- ((8S)-8-(1-(2- methoxyethyl)-4- methyl-1H-pyrazol-5- yl)-5- azaspiro[2.5]octan-5- yl)-2-(trifluoromethyl)- 4-pyrimidinyl)-2- methyl-3-azetidinyl)-1- piperazinyl)-2-propen-1- one | E-1 | 603.2 |
| 2-226 | 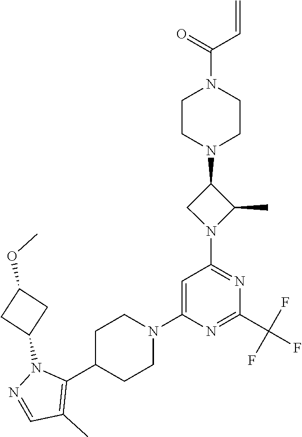 [SMILES: C=CC(=O)N1CCN([C@@H]2CN(c3cc(N4CCC(c5c(C)cnn5[C@H]5C[C@@H](OC)C5)CC4)nc(C(F)(F)F)n3)[C@@H]2C)CC1] | 1-(4-((2R,3R)-1-(6-(4- (1-(cis-3- methoxycyclobutyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 603.3 |
| 2-227 | 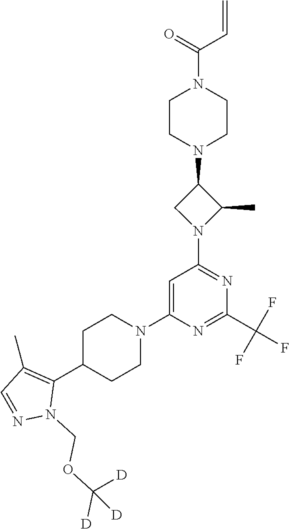 [SMILES: [2H]C([2H])([2H])OCn1ncc(C)c1C1CCN(c2cc(N3C[C@@H](N4CCN(C(=O)C=C)CC4)[C@H]3C)nc(C(F)(F)F)n2)CC1] | 1-(4-((2R,3R)-1-(6-(4- (1-(2-(methoxy- d3)ethyl)-4-methyl-1H- pyrazol-5-yl)piperidin- 1-yl)-2- (trifluoromethyl)pyrimidin- 4-yl)-2- methylazetidin-3- yl)piperazin-1-yl)prop- 2-en-1-one | E-1 | 580.3 |
| 2-228 | 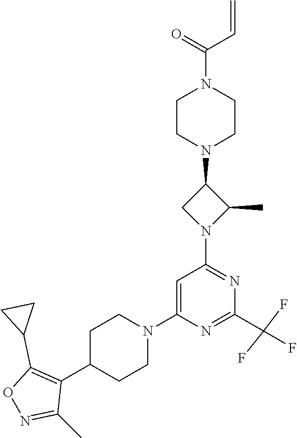 [SMILES: C=CC(=O)N1CCN([C@@H]2CN(c3cc(N4CCC(c5c(C)noc5C5CC5)CC4)nc(C(F)(F)F)n3)[C@@H]2C)CC1] | 1-(4-((2R,3R)-1-(6-(4- (5-cyclopropyl-3- methyl-1,2-oxazol-4-yl)- 1-piperidinyl)-2- (trifluoromethyl)-4- pyrimidinyl)-2-methyl- 3-azetidinyl)-1- piperazinyl)-2-propen-1- one | E-1 | 560.2 |
| 2-229 | 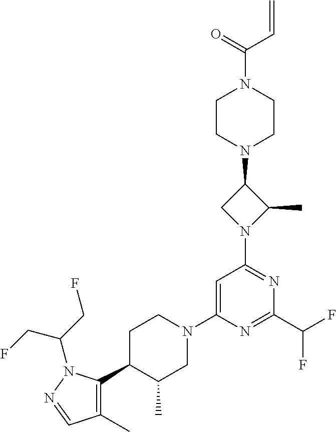 [SMILES: C=CC(=O)N1CCN([C@@H]2CN(c3cc(N4CC[C@H](c5c(C)cnn5C(CF)CF)[C@@H](C)C4)nc(C(F)F)n3)[C@@H]2C)CC1] | 1-(4-((2R,3R)-1-(2- (difluoromethyl)-6- ((3R,4S)-4-(1-(1,3- difluoro-2-propanyl)-4- methyl-1H-pyrazol-5- yl)-3-methyl-1- piperidinyl)-4- pyrimidinyl)-2-methyl- 3-azetidinyl)-1- piperazinyl)-2-propen-1- one | E-2 | 593.2 |
| | mixture of trans isomers at piperidine | | | |
| 2-230 | 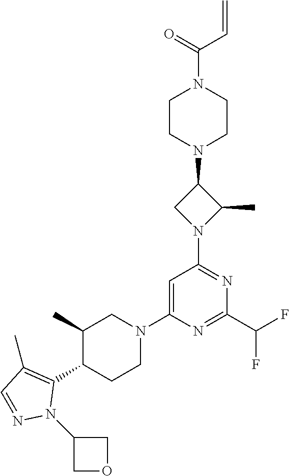 [SMILES: C=CC(=O)N1CCN([C@@H]2CN(c3cc(N4CC[C@H](c5c(C)cnn5C5COC5)[C@@H](C)C4)nc(C(F)F)n3)[C@@H]2C)CC1] | 1-(4-((2R,3R)-1-(2- (difluoromethyl)-6- ((3R,4S)-3-methyl-4-(4- methyl-1-(3-oxetanyl)- 1H-pyrazol-5-yl)-1- piperidinyl)-4- pyrimidinyl)-2-methyl- 3-azetidinyl)-1- piperazinyl)-2-propen-1- one | E-2 | 571.2 |
| | mixture of trans isomers at piperidine | | | |
| 2-231 | 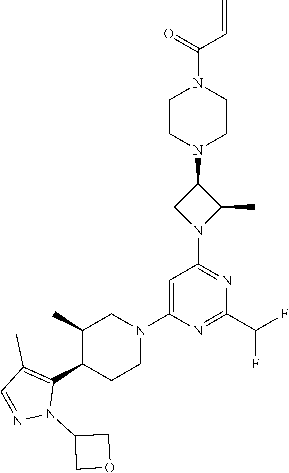 [SMILES: C=CC(=O)N1CCN([C@@H]2CN(c3cc(N4CC[C@@H](c5c(C)cnn5C5COC5)[C@@H](C)C4)nc(C(F)F)n3)[C@@H]2C)CC1] | 1-(4-((2R,3R)-1-(2- (difluoromethyl)-6- ((3R,4R)-3-methyl-4-(4- methyl-1-(3-oxetanyl)- 1H-pyrazol-5-yl)-1- piperidinyl)-4- pyrimidinyl)-2-methyl- 3-azetidinyl)-1- piperazinyl)-2-propen-1- one | E-1 | 593.2 |
| | mixture of cis isomers at piperidine | | | |
| 2-232 | 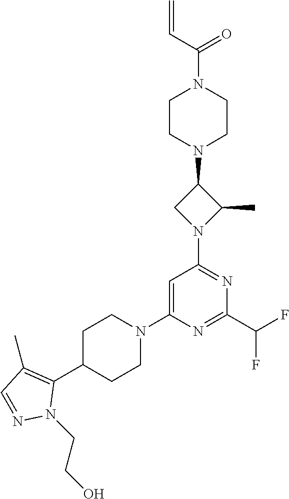 [SMILES: C=CC(=O)N1CCN([C@@H]2CN(c3cc(N4CCC(c5c(C)cnn5CCO)CC4)nc(C(F)F)n3)[C@@H]2C)CC1] | 1-(4-((2R,3R)-1-(2- (difluoromethyl)-6-(4- (1-(2-hydroxyethyl)-4- methyl-1H-pyrazol-5- yl)-1-piperidinyl)-4- pyrimidinyl)-2-methyl- 3-azetidinyl)-1- piperazinyl)-2-propen-1- one | E-2 | 545.4 |
| 2-233 | 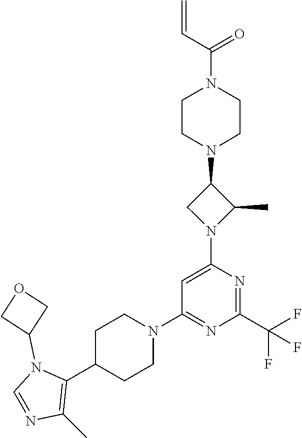 [SMILES: C=CC(=O)N1CCN([C@@H]2CN(c3cc(N4CCC(c5c(C)ncn5C5COC5)CC4)nc(C(F)(F)F)n3)[C@@H]2C)CC1] | 1-(4-((2R,3R)-2-methyl- 1-(6-(4-(4-methyl-1-(3- oxetanyl)-1H-imidazol- 5-yl)-1-piperidinyl)-2- (trifluoromethyl)-4- pyrimidinyl)-3- azetidinyl)-1- piperazinyl)-2-propen-1- one | E-8 | 575.3 |
| 2-234 | 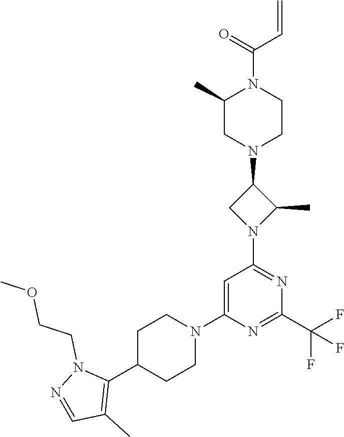 [SMILES: C=CC(=O)N1CCN([C@@H]2CN(c3cc(N4CCC(c5c(C)cnn5CCOC)CC4)nc(C(F)(F)F)n3)[C@@H]2C)C[C@H]1C] | 1-((2R)-4-((2R,3R)-1- (6-(4-(1-(2- methoxyethyl)-4- methyl-1H-pyrazol-5- yl)-1-piperidinyl)-2- (trifluoromethyl)-4- pyrimidinyl)-2-methyl- 3-azetidinyl)-2-methyl- 1-piperazinyl)-2-propen- 1-one | E-6 | 591.3 |
| 2-235 | 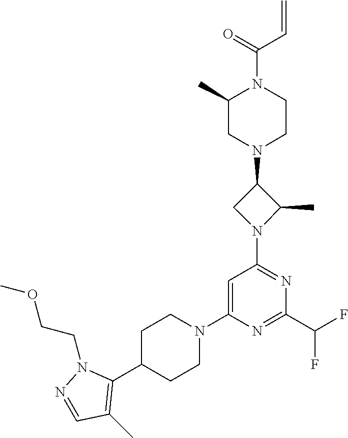 [SMILES: C=CC(=O)N1CCN([C@@H]2CN(c3cc(N4CCC(c5c(C)cnn5CCOC)CC4)nc(C(F)F)n3)[C@@H]2C)C[C@H]1C] | 1-((2R)-4-((2R,3R)-1- (2-(difluoromethyl)-6- (4-(1-(2-methoxyethyl)- 4-methyl-1H-pyrazol-5- yl)-1-piperidinyl)-4- pyrimidinyl)-2-methyl- 3-azetidinyl)-2-methyl- 1-piperazinyl)-2-propen- 1-one | E-11 | 573.3 |
| 2-236 | 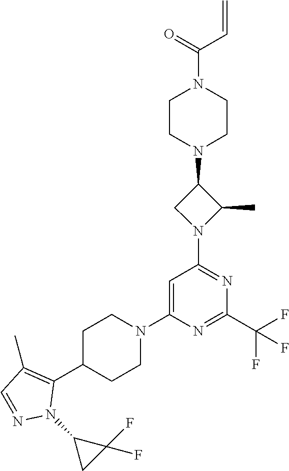 [SMILES: C=CC(=O)N1CCN([C@@H]2CN(c3cc(N4CCC(c5c(C)cnn5[C@H]5CC5(F)F)CC4)nc(C(F)(F)F)n3)[C@@H]2C)CC1] | 1-(4-((2R,3R)-1-(6-(4- (1-((1S)-2,2- difluorocyclopropyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-8 | 595.2 |
| 2-237 | 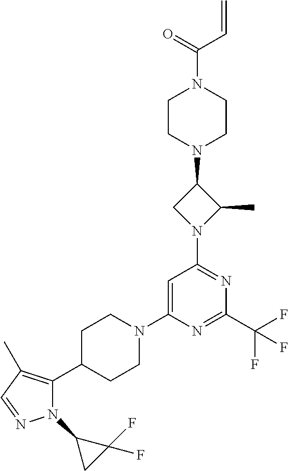 [SMILES: C=CC(=O)N1CCN([C@@H]2CN(c3cc(N4CCC(c5c(C)cnn5[C@@H]5CC5(F)F)CC4)nc(C(F)(F)F)n3)[C@@H]2C)CC1] | 1-(4-((2R,3R)-1-(6-(4- (1-((1R)-2,2- difluorocyclopropyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-8 | 595.2 |
| 2-238 | 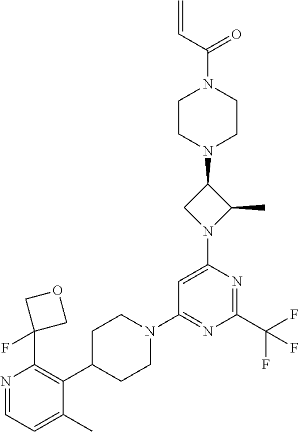 [SMILES: C=CC(=O)N1CCN([C@@H]2CN(c3cc(N4CCC(c5c(C)ccnc5C5(F)COC5)CC4)nc(C(F)(F)F)n3)[C@@H]2C)CC1] | 1-(4-((2R,3R)-1-(6-(4- (2-(3-fluoro-3- oxetanyl)-4-methyl-3- pyridinyl)-1- piperidinyl)-2- (trifluoromethyl)-4- pyrimidinyl)-2-methyl- 3-azetidinyl)-1- piperazinyl)-2-propen-1- one | E-1 | 604.3 |
| 2-239 | 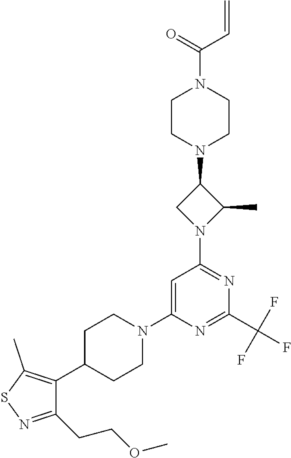 [SMILES: C=CC(=O)N1CCN([C@@H]2CN(c3cc(N4CCC(c5c(CCOC)nsc5C)CC4)nc(C(F)(F)F)n3)[C@@H]2C)CC1] | 1-(4-((2R,3R)-1-(6-(4- (3-(2-methoxyethyl)-5- methyl-1,2-thiazol-4- yl)-1-piperidinyl)-2- (trifluoromethyl)-4- pyrimidinyl)-2-methyl- 3-azetidinyl)-1- piperazinyl)-2-propen-1- one | E-1 | 594.2 |
| 2-240 | 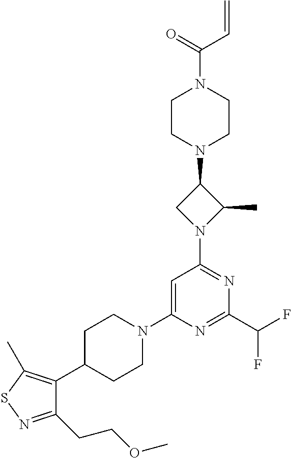 [SMILES: C=CC(=O)N1CCN([C@@H]2CN(c3cc(N4CCC(c5c(CCOC)nsc5C)CC4)nc(C(F)F)n3)[C@@H]2C)CC1] | 1-(4-((2R,3R)-1-(2- (difluoromethyl)-6-(4- (3-(2-methoxyethyl)-5- methyl-1,2-thiazol-4- yl)-1-piperidinyl)-4- pyrimidinyl)-2-methyl- 3-azetidinyl)-1- piperazinyl)-2-propen-1- one | E-2 | 576.2 |
| 2-241 | 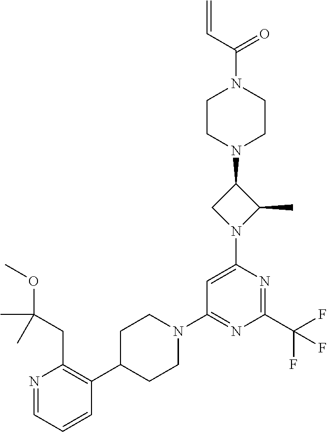 [SMILES: C=CC(=O)N1CCN([C@@H]2CN(c3cc(N4CCC(c5cccnc5CC(C)(C)OC)CC4)nc(C(F)(F)F)n3)[C@@H]2C)CC1] | 1-(4-((2R,3R)-1-(6-(4- (2-(2-methoxy-2- methylpropyl)-3- pyridinyl)-1- piperidinyl)-2- (trifluoromethyl)-4- pyrimidinyl)-2-methyl- 3-azetidinyl)-1- piperazinyl)-2-propen-1- one | E-1 | 602.1 |
| 2-242 | 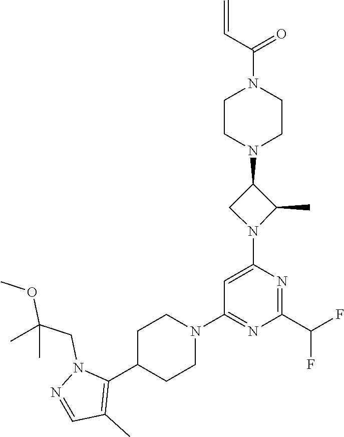 [SMILES: C=CC(=O)N1CCN([C@@H]2CN(c3cc(N4CCC(c5c(C)cnn5CC(C)(C)OC)CC4)nc(C(F)F)n3)[C@@H]2C)CC1] | 1-(4-((2R,3R)-1-(2- (difluoromethyl)-6-(4- (1-(2-methoxy-2- methylpropyl)-4-methyl- 1H-pyrazol-5-yl)-1- piperidinyl)-4- pyrimidinyl)-2-methyl- 3-azetidinyl)-1- piperazinyl)-2-propen-1- one | E-2 | 587.3 |
| 2-243 | 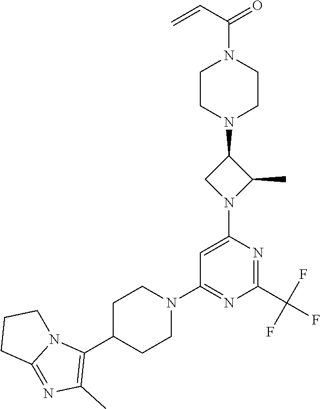 [SMILES: C=CC(=O)N1CCN([C@@H]2CN(c3cc(N4CCC(c5c(C)nc6n5CCC6)CC4)nc(C(F)(F)F)n3)[C@@H]2C)CC1] | 1-(4-((2R,3R)-2-methyl- 1-(6-(4-(2-methyl-6,7- dihydro-5H-pyrrolo[1,2- a]imidazol-3-yl)-1- piperidinyl)-2- (trifluoromethyl)-4- pyrimidinyl)-3- azetidinyl)-1- piperazinyl)-2-propen-1- one | E-1 | 559.3 |
| 2-244 | 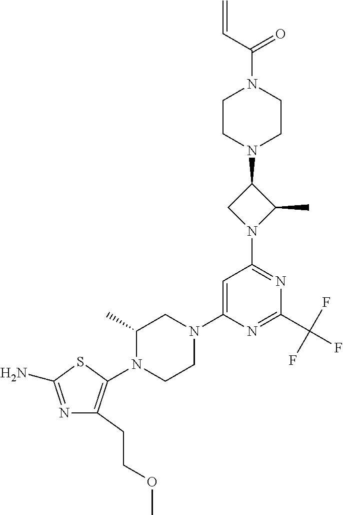 [SMILES: C=CC(=O)N1CCN([C@@H]2CN(c3cc(N4CCN(c5sc(N)nc5CCOC)[C@H](C)C4)nc(C(F)(F)F)n3)[C@@H]2C)CC1] | 1-(4-((2R,3R)-1-(6- ((3R)-4-(2-amino-4-(2- methoxyethyl)-1,3- thiazol-5-yl)-3-methyl- 1-piperazinyl)-2- (trifluoromethyl)-4- pyrimidinyl)-2-methyl- 3-azetidinyl)-1- piperazinyl)-2-propen-1- one | E-1 | 610.2 |
| 2-245 | 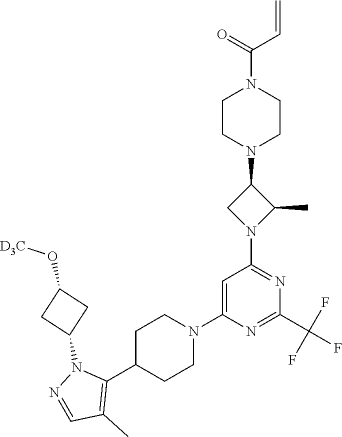 [SMILES: [2H]C([2H])([2H])O[C@H]1C[C@@H](n2ncc(C)c2C2CCN(c3cc(N4C[C@@H](N5CCN(C(=O)C=C)CC5)[C@H]4C)nc(C(F)(F)F)n3)CC2)C1] | 1-(4-((2R,3R)-2-methyl- 1-(6-(4-(4-methyl-1- (cis-3- ((~2~H_3_)methyloxy) cyclobutyl)-1H-pyrazol- 5-yl)-1-piperidinyl)-2- (trifluoromethyl)-4- pyrimidinyl)-3- azetidinyl)-1- piperazinyl)-2-propen-1- one | E-8 | 606.3 |
| 2-246 | 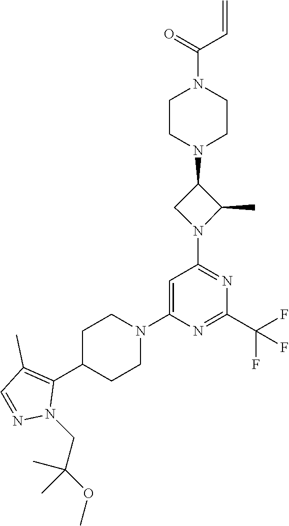 [SMILES: C=CC(=O)N1CCN([C@@H]2CN(c3cc(N4CCC(c5c(C)cnn5CC(C)(C)OC)CC4)nc(C(F)(F)F)n3)[C@@H]2C)CC1] | 1-(4-((2R,3R)-1-(6-(4- (1-(2-methoxy-2- methylpropyl)-4-methyl- 1H-pyrazol-5-yl)-1- piperidinyl)-2- (trifluoromethyl)-4- pyrimidinyl)-2-methyl- 3-azetidinyl)-1- piperazinyl)-2-propen-1- one | E-1 | 605.4 |
| 2-247 | 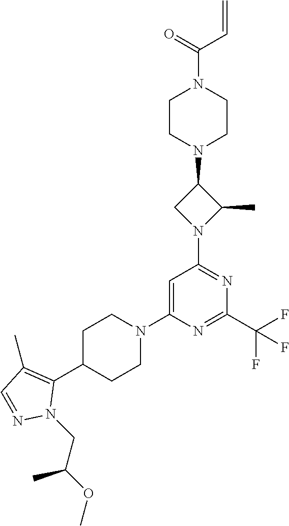 [SMILES: C=CC(=O)N1CCN([C@@H]2CN(c3cc(N4CCC(c5c(C)cnn5C[C@H](C)OC)CC4)nc(C(F)(F)F)n3)[C@@H]2C)CC1] | 1-(4-((2R,3R)-1-(6-(4- (1-((2S)-2- methoxypropyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 591.3 |
| 2-248 | 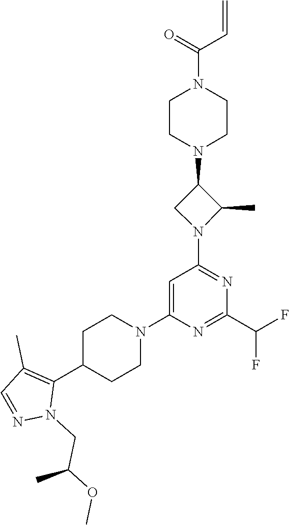 [SMILES: C=CC(=O)N1CCN([C@@H]2CN(c3cc(N4CCC(c5c(C)cnn5C[C@H](C)OC)CC4)nc(C(F)F)n3)[C@@H]2C)CC1] | 1-(4-((2R,3R)-1-(2- (difluoromethyl)-6-(4- (1-((2S)-2- methoxypropyl)-4- methyl-1H-pyrazol-5- yl)-1-piperidinyl)-4- pyrimidinyl)-2-methyl- 3-azetidinyl)-1- piperazinyl)-2-propen-1- one | E-2 | 573.3 |
| 2-249 | 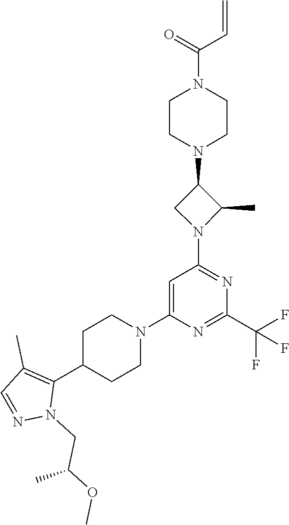 [SMILES: C=CC(=O)N1CCN([C@@H]2CN(c3cc(N4CCC(c5c(C)cnn5C[C@@H](C)OC)CC4)nc(C(F)(F)F)n3)[C@@H]2C)CC1] | 1-(4-((2R,3R)-1-(6-(4- (1-((2R)-2- methoxypropyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-2 | 591.3 |
| 2-250 | 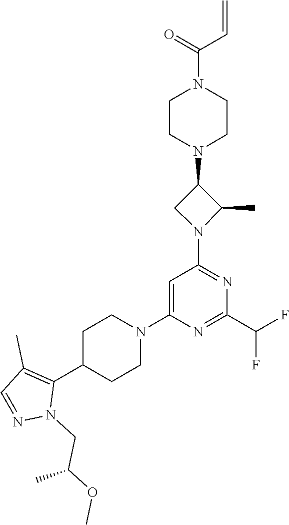 [SMILES: C=CC(=O)N1CCN([C@@H]2CN(c3cc(N4CCC(c5c(C)cnn5C[C@@H](C)OC)CC4)nc(C(F)F)n3)[C@@H]2C)CC1] | 1-(4-((2R,3R)-1-(2- (difluoromethyl)-6-(4- (1-((2R)-2- methoxypropyl)-4- methyl-1H-pyrazol-5- yl)-1-piperidinyl)-4- pyrimidinyl)-2-methyl- 3-azetidinyl)-1- piperazinyl)-2-propen-1- one | E-2 | 573.3 |
| 2-251 | 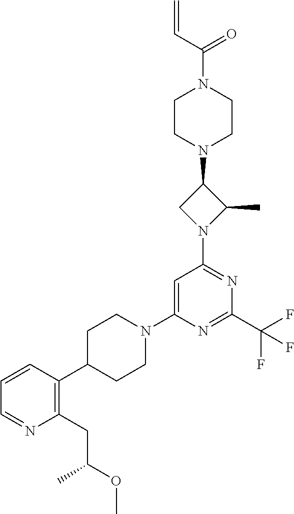 [SMILES: C=CC(=O)N1CCN([C@@H]2CN(c3cc(N4CCC(c5cccnc5C[C@@H](C)OC)CC4)nc(C(F)(F)F)n3)[C@@H]2C)CC1] | 1-(4-((2R,3R)-1-(6-(4- (2-((2R)-2- methoxypropyl)-3- pyridinyl)-1- piperidinyl)-2- (trifluoromethyl)-4- pyrimidinyl)-2-methyl- 3-azetidinyl)-1- piperazinyl)-2-propen-1- one | E-1 | 588.5 |
| 2-252 | 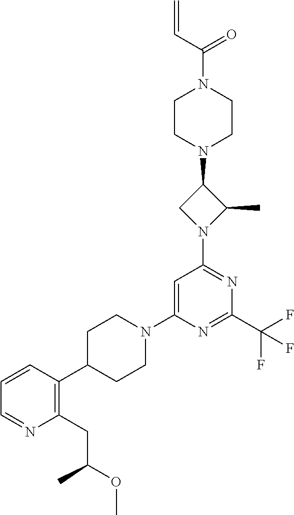 [SMILES: C=CC(=O)N1CCN([C@@H]2CN(c3cc(N4CCC(c5cccnc5C[C@H](C)OC)CC4)nc(C(F)(F)F)n3)[C@@H]2C)CC1] | 1-(4-((2R,3R)-1-(6-(4- (2-((2S)-2- methoxypropyl)-3- pyridinyl)-1- piperidinyl)-2- (trifluoromethyl)-4- pyrimidinyl)-2-methyl- 3-azetidinyl)-1- piperazinyl)-2-propen-1- one | E-1 | 588.5 |
| 2-253 | 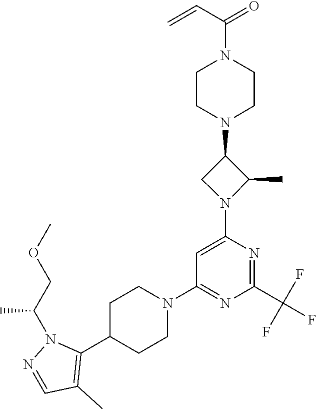 [SMILES: C=CC(=O)N1CCN([C@@H]2CN(c3cc(N4CCC(c5c(C)cnn5[C@H](C)COC)CC4)nc(C(F)(F)F)n3)[C@@H]2C)CC1] | 1-(4-((2R,3R)-1-(6-(4- (1-((2R)-1-methoxy-2- propanyl)-4-methyl-1H- pyrazol-5-yl)-1- piperidinyl)-2- (trifluoromethyl)-4- pyrimidinyl)-2-methyl- 3-azetidinyl)-1- piperazinyl)-2-propen-1- one | E-1 | 591.1 |
| 2-254 | 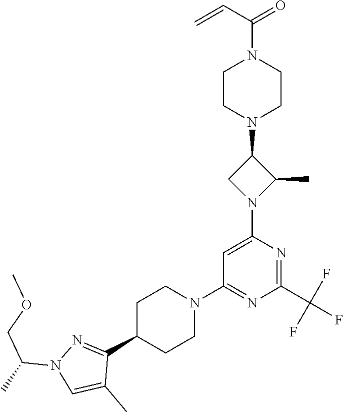 | 1-(4-((2R,3R)-1-(6-(4- (1-((2R)-1-methoxy-2- propanyl)-4-methyl-1H- pyrazol-3-yl)-1- piperidinyl)-2- (trifluoromethyl)-4- pyrimidinyl)-2-methyl- 3-azetidinyl)-1- piperazinyl)-2-propen-1- one | E-1 | 591.1 |
| 2-255 | 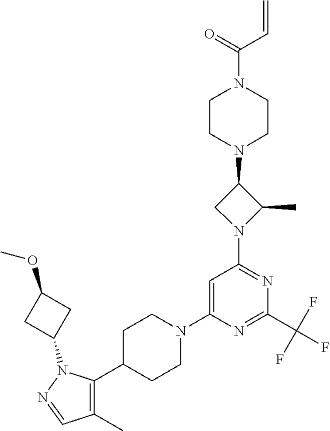 [SMILES: C=CC(=O)N1CCN([C@@H]2CN(c3cc(N4CCC(c5c(C)cnn5[C@H]5C[C@H](OC)C5)CC4)nc(C(F)(F)F)n3)[C@@H]2C)CC1] | 1-(4-((2R,3R)-1-(6-(4- (1-(trans-3- methoxycyclobutyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 603.1 |
| 2-256 | 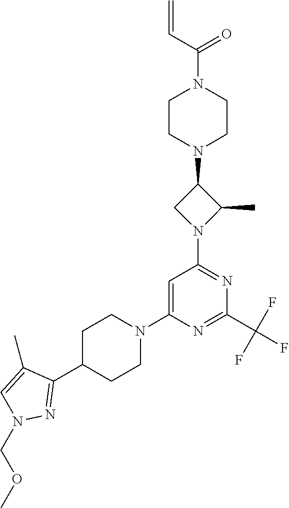 [SMILES: C=CC(=O)N1CCN([C@@H]2CN(c3cc(N4CCC(c5nn(COC)cc5C)CC4)nc(C(F)(F)F)n3)[C@@H]2C)CC1] | 1-(4-((2R,3R)-1-(6-(4- (1-(methoxymethyl)-4- methyl-1H-pyrazol-3- yl)-1-piperidinyl)-2- (trifluoromethyl)-4- pyrimidinyl)-2-methyl- 3-azetidinyl)-1- piperazinyl)-2-propen-1- one | E-1 | 563.2 |
| 2-257 | 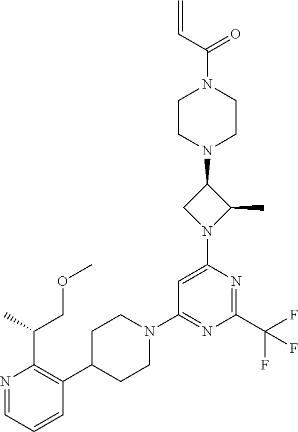 [SMILES: C=CC(=O)N1CCN([C@@H]2CN(c3cc(N4CCC(c5cccnc5[C@H](C)COC)CC4)nc(C(F)(F)F)n3)[C@@H]2C)CC1] | 1-(4-((2R,3R)-1-(6-(4- (2-((2S)-1-methoxy-2- propanyl)-3-pyridinyl)- 1-piperidinyl)-2- (trifluoromethyl)-4- pyrimidinyl)-2-methyl- 3-azetidinyl)-1- piperazinyl)-2-propen-1- one | E-1 | 588.3 |
| 2-258 | 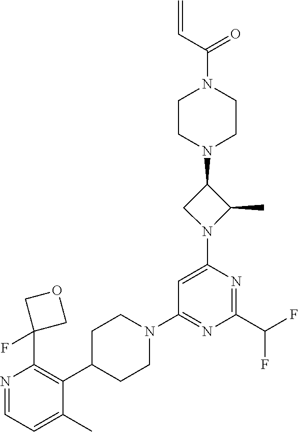 [SMILES: C=CC(=O)N1CCN([C@@H]2CN(c3cc(N4CCC(c5c(C)ccnc5C5(F)COC5)CC4)nc(C(F)F)n3)[C@@H]2C)CC1] | 1-(4-((2R,3R)-1-(2- (difluoromethyl)-6-(4- (2-(3-fluoro-3- oxetanyl)-4-methyl-3- pyridinyl)-1- piperidinyl)-4- pyrimidinyl)-2-methyl- 3-azetidinyl)-1- piperazinyl)-2-propen-1- one | E-2 | 586.3 |
| 2-259 | 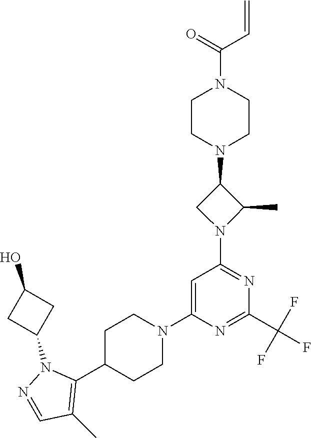 [SMILES: C=CC(=O)N1CCN([C@@H]2CN(c3cc(N4CCC(c5c(C)cnn5[C@H]5C[C@H](O)C5)CC4)nc(C(F)(F)F)n3)[C@@H]2C)CC1] | 1-(4-((2R,3R)-1-(6-(4- (1-(trans-3- hydroxycyclobutyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 589.2 |
| 2-260 | 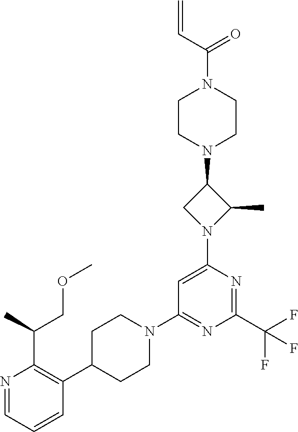 [SMILES: C=CC(=O)N1CCN([C@@H]2CN(c3cc(N4CCC(c5cccnc5[C@@H](C)COC)CC4)nc(C(F)(F)F)n3)[C@@H]2C)CC1] | 1-(4-((2R,3R)-1-(6-(4- (2-((2R)-1-methoxy-2- propanyl)-3-pyridinyl)- 1-piperidinyl)-2- (trifluoromethyl)-4- pyrimidinyl)-2-methyl- 3-azetidinyl)-1- piperazinyl)-2-propen-1- one | E-1 | 588.3 |
| 2-261 | 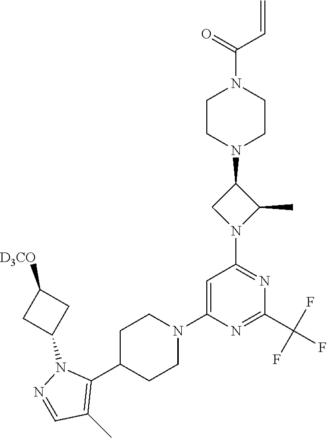 | 1-(4-((2R,3R)-1-(6-(4- (1-((1r,3R)-3-(methoxy- d3)cyclobutyl)-4- methyl-1H-pyrazol-5- yl)piperidin-1-yl)-2- (trifluoromethyl)pyrimidin- 4-yl)-2- methylazetidin-3- yl)piperazin-1-yl)prop- 2-en-1-one | E-8 | 606.1 |
| 2-262 | 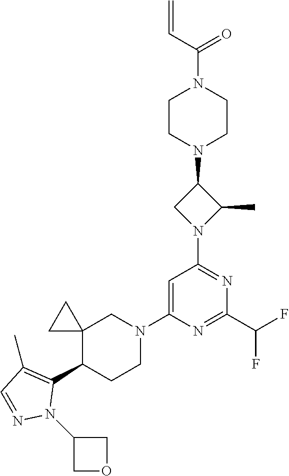 [SMILES: C=CC(=O)N1CCN([C@@H]2CN(c3cc(N4CC[C@@H](c5c(C)cnn5C5COC5)C5(CC5)C4)nc(C(F)F)n3)[C@@H]2C)CC1] | 1-(4-((2R,3R)-1-(2- (difluoromethyl)-6- ((8R)-8-(4-methyl-1-(3- oxetanyl)-1H-pyrazol-5- yl)-5- azaspiro[2.5]octan-5- yl)-4-pyrimidinyl)-2- methyl-3-azetidinyl)-1- piperazinyl)-2-propen-1- one | E-9 | 583.2 |
| 2-264 | 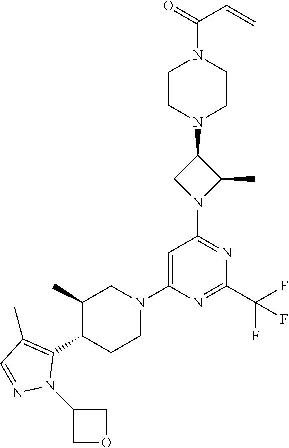 [SMILES: C=CC(=O)N1CCN([C@@H]2CN(c3cc(N4CC[C@H](c5c(C)cnn5C5COC5)[C@@H](C)C4)nc(C(F)(F)F)n3)[C@@H]2C)CC1] | 1-(4-((2R,3R)-2-methyl- 1-(6-((3R,4S)-3-methyl- 4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-8 | 589.2 |
| 2-265 | 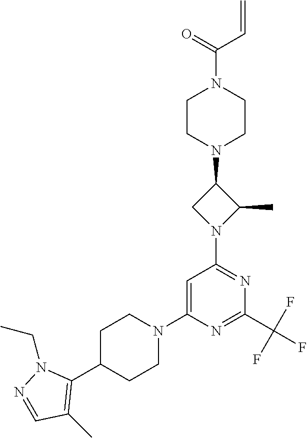 [SMILES: C=CC(=O)N1CCN([C@@H]2CN(c3cc(N4CCC(c5c(C)cnn5CC)CC4)nc(C(F)(F)F)n3)[C@@H]2C)CC1] | 1-(4-((2R,3R)-1-(6-(4- (1-ethyl-4-methyl-1H- pyrazol-5-yl)-1- piperidinyl)-2- (trifluoromethyl)-4- pyrimidinyl)-2-methyl- 3-azetidinyl)-1- piperazinyl)-2-propen-1- one | E-1 | 547.3 |
| 2-266 | 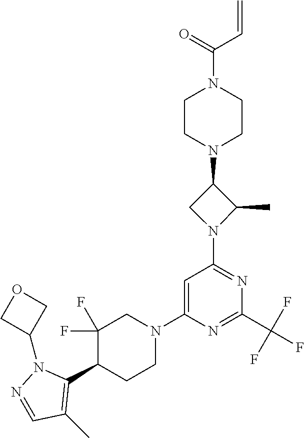 [SMILES: C=CC(=O)N1CCN([C@@H]2CN(c3cc(N4CC[C@@H](c5c(C)cnn5C5COC5)C(F)(F)C4)nc(C(F)(F)F)n3)[C@@H]2C)CC1] | 1-(4-((2R,3R)-1-(6- ((4S)-3,3-difluoro-4-(4- methyl-1-(3-oxetanyl)- 1H-pyrazol-5-yl)-1- piperidinyl)-2- (trifluoromethyl)-4- pyrimidinyl)-2-methyl- 3-azetidinyl)-1- piperazinyl)-2-propen-1- one | E-1 | 611.3 |
| 2-267 | 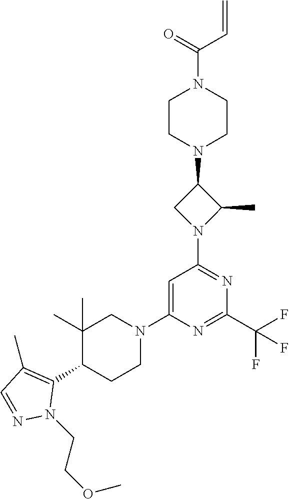 [SMILES: C=CC(=O)N1CCN([C@@H]2CN(c3cc(N4CC[C@H](c5c(C)cnn5CCOC)C(C)(C)C4)nc(C(F)(F)F)n3)[C@@H]2C)CC1] | 1-(4-((2R,3R)-1-(6- ((4S)-4-(1-(2- methoxyethyl)-4- methyl-1H-pyrazol-5- yl)-3,3-dimethyl-1- piperidinyl)-2- (trifluoromethyl)-4- pyrimidinyl)-2-methyl- 3-azetidinyl)-1- piperazinyl)-2-propen-1- one | E-1 | 605.0 |
| 2-268 | 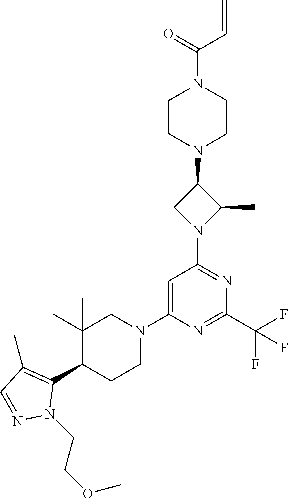 [SMILES: C=CC(=O)N1CCN([C@@H]2CN(c3cc(N4CC[C@@H](c5c(C)cnn5CCOC)C(C)(C)C4)nc(C(F)(F)F)n3)[C@@H]2C)CC1] | 1-(4-((2R,3R)-1-(6- ((4R)-4-(1-(2- methoxyethyl)-4- methyl-1H-pyrazol-5- yl)-3,3-dimethyl-1- piperidinyl)-2- (trifluoromethyl)-4- pyrimidinyl)-2-methyl- 3-azetidinyl)-1- piperazinyl)-2-propen-1- one | E-1 | 605.0 |
| 2-269 | 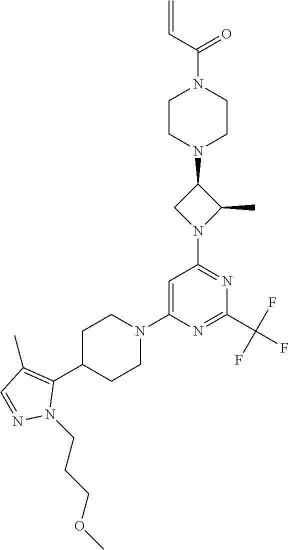 [SMILES: C=CC(=O)N1CCN([C@@H]2CN(c3cc(N4CCC(c5c(C)cnn5CCCOC)CC4)nc(C(F)(F)F)n3)[C@@H]2C)CC1] | 1-(4-((2R,3R)-1-(6-(4- (1-(3-methoxypropyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-8 | 592.0 |
| 2-270 | 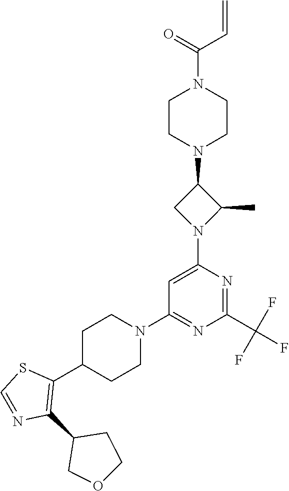 [SMILES: C=CC(=O)N1CCN([C@@H]2CN(c3cc(N4CCC(c5scnc5[C@H]5CCOC5)CC4)nc(C(F)(F)F)n3)[C@@H]2C)CC1] | 1-(4-((2R,3R)-2-methyl- 1-(6-(4-(4-((3R)- tetrahydro-3-furanyl)- 1,3-thiazol-5-yl)-1- piperidinyl)-2- (trifluoromethyl)-4- pyrimidinyl)-3- azetidinyl)-1- piperazinyl)-2-propen-1- one | E-8 | 592.0 |
| 2-271 | 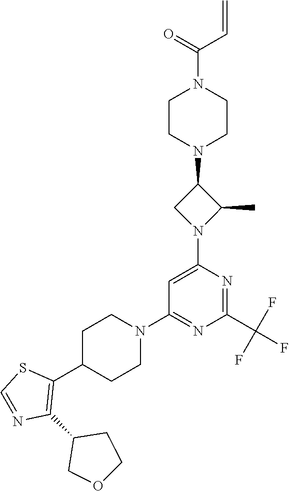 [SMILES: C=CC(=O)N1CCN([C@@H]2CN(c3cc(N4CCC(c5scnc5[C@@H]5CCOC5)CC4)nc(C(F)(F)F)n3)[C@@H]2C)CC1] | 1-(4-((2R,3R)-2-methyl- 1-(6-(4-(4-((3S)- tetrahydro-3-furanyl)- 1,3-thiazol-5-yl)-1- piperidinyl)-2- (trifluoromethyl)-4- pyrimidinyl)-3- azetidinyl)-1- piperazinyl)-2-propen-1- one | E-1 | 592.0 |
| 2-272 | 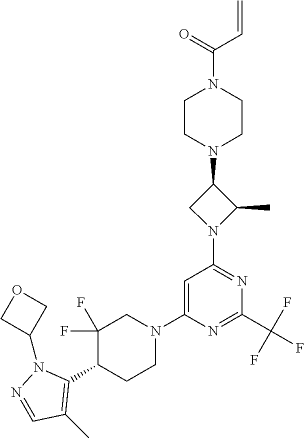 [SMILES: C=CC(=O)N1CCN([C@@H]2CN(c3cc(N4CC[C@H](c5c(C)cnn5C5COC5)C(F)(F)C4)nc(C(F)(F)F)n3)[C@@H]2C)CC1] | 1-(4-((2R,3R)-1-(6- ((4R)-3,3-difluoro-4-(4- methyl-1-(3-oxetanyl)- 1H-pyrazol-5-yl)-1- piperidinyl)-2- (trifluoromethyl)-4- pyrimidinyl)-2-methyl- 3-azetidinyl)-1- piperazinyl)-2-propen-1- one | E-1 | 611.3 |
| 2-273 | 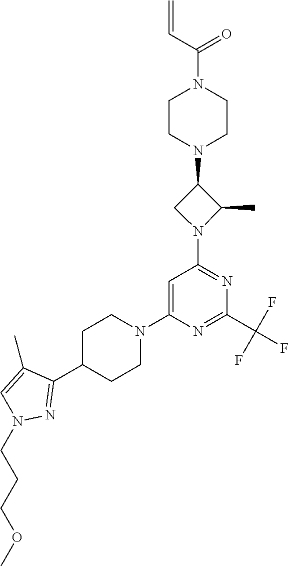 [SMILES: C=CC(=O)N1CCN([C@@H]2CN(c3cc(N4CCC(c5nn(CCCOC)cc5C)CC4)nc(C(F)(F)F)n3)[C@@H]2C)CC1] | 1-(4-((2R,3R)-1-(6-(4- (1-(3-methoxypropyl)-4- methyl-1H-pyrazol-3- yl)-1-piperidinyl)-2- (trifluoromethyl)-4- pyrimidinyl)-2-methyl- 3-azetidinyl)-1- piperazinyl)-2-propen-1- one | E-1 | 591.2 |
| 2-274 | 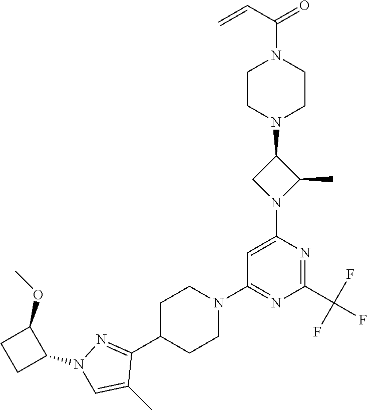 [SMILES: C=CC(=O)N1CCN([C@@H]2CN(c3cc(N4CCC(c5nn([C@@H]6CC[C@H]6OC)cc5C)CC4)nc(C(F)(F)F)n3)[C@@H]2C)CC1] | 1-(4-((2R,3R)-1-(6-(4- (1-((1R,2R)-2- methoxycyclobutyl)-4- methyl-1H-pyrazol-3- yl)-1-piperidinyl)-2- (trifluoromethyl)-4- pyrimidinyl)-2-methyl- 3-azetidinyl)-1- piperazinyl)-2-propen-1- one | E-8 | 603.4 |
| 2-275 | 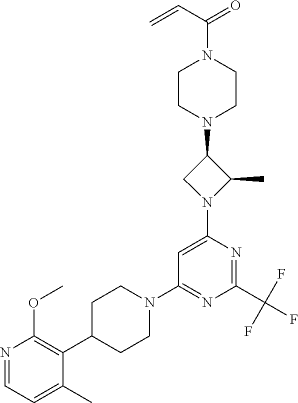 [SMILES: C=CC(=O)N1CCN([C@@H]2CN(c3cc(N4CCC(c5c(C)ccnc5OC)CC4)nc(C(F)(F)F)n3)[C@@H]2C)CC1] | 1-(4-((2R,3R)-1-(6-(4- (2-methoxy-4-methyl-3- pyridinyl)-1- piperidinyl)-2- (trifluoromethyl)-4- pyrimidinyl)-2-methyl- 3-azetidinyl)-1- piperazinyl)-2-propen-1- one | E-1 | 560.1 |
| 2-276 | 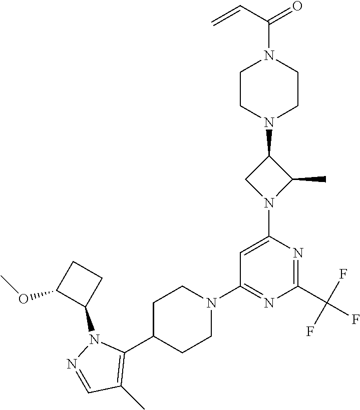 [SMILES: C=CC(=O)N1CCN([C@@H]2CN(c3cc(N4CCC(c5c(C)cnn5[C@@H]5CC[C@H]5OC)CC4)nc(C(F)(F)F)n3)[C@@H]2C)CC1] | 1-(4-((2R,3R)-1-(6-(4- (1-((1R,2R)-2- methoxycyclobutyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-8 | 603.4 |
| 2-277 | 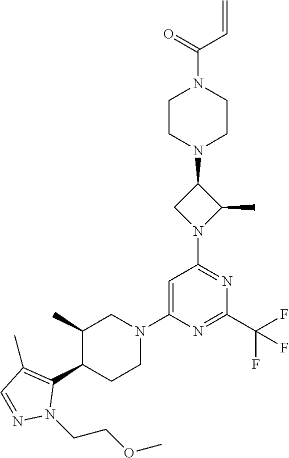 [SMILES: C=CC(=O)N1CCN([C@@H]2CN(c3cc(N4CC[C@@H](c5c(C)cnn5CCOC)[C@@H](C)C4)nc(C(F)(F)F)n3)[C@@H]2C)CC1] | 1-(4-((2R,3R)-1-(6- ((3R,4R)-4-(1-(2- methoxyethyl)-4- methyl-1H-pyrazol-5- yl)-3-methyl-1- piperidinyl)-2- (trifluoromethyl)-4- pyrimidinyl)-2-methyl- 3-azetidinyl)-1- piperazinyl)-2-propen-1- one | E-1 | 591.0 |
| 2-278 | 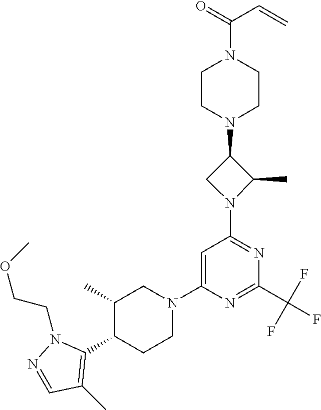 [SMILES: C=CC(=O)N1CCN([C@@H]2CN(c3cc(N4CC[C@H](c5c(C)cnn5CCOC)[C@H](C)C4)nc(C(F)(F)F)n3)[C@@H]2C)CC1] | 1-(4-((2R,3R)-1-(6- ((3S,4S)-4-(1-(2- methoxyethyl)-4- methyl-1H-pyrazol-5- yl)-3-methyl-1- piperidinyl)-2- (trifluoromethyl)-4- pyrimidinyl)-2-methyl- 3-azetidinyl)-1- piperazinyl)-2-propen-1- one | E-1 | 591.0 |
| 2-279 | 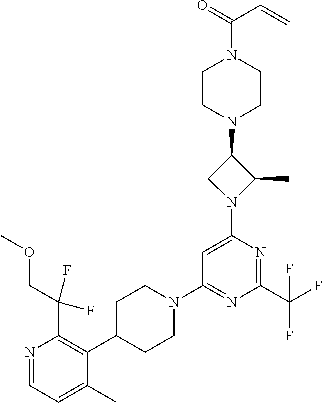 [SMILES: C=CC(=O)N1CCN([C@@H]2CN(c3cc(N4CCC(c5c(C)ccnc5C(F)(F)COC)CC4)nc(C(F)(F)F)n3)[C@@H]2C)CC1] | 1-(4-((2R,3R)-1-(6-(4- (2-(1,1-difluoro-2- methoxyethyl)-4- methyl-3-pyridinyl)-1- piperidinyl)-2- (trifluoromethyl)-4- pyrimidinyl)-2-methyl- 3-azetidinyl)-1- piperazinyl)-2-propen-1- one | E-1 | 624.2 |
| 2-280 | 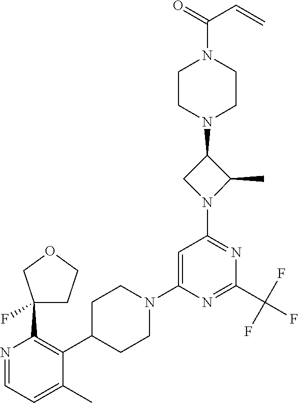 [SMILES: C=CC(=O)N1CCN([C@@H]2CN(c3cc(N4CCC(c5c(C)ccnc5[C@]5(F)CCOC5)CC4)nc(C(F)(F)F)n3)[C@@H]2C)CC1] | 1-(4-((2R,3R)-1-(6-(4- (2-((3R)-3- fluorotetrahydro-3- furanyl)-4-methyl-3- pyridinyl)-1- piperidinyl)-2- (trifluoromethyl)-4- pyrimidinyl)-2-methyl- 3-azetidinyl)-1- piperazinyl)-2-propen-1- one | E-1 | 618.3 |
| 2-281 | 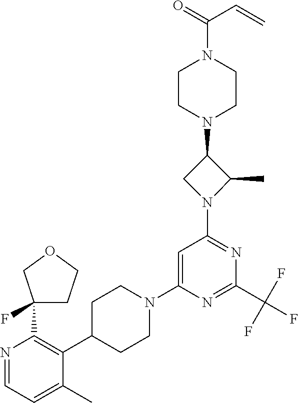 [SMILES: C=CC(=O)N1CCN([C@@H]2CN(c3cc(N4CCC(c5c(C)ccnc5[C@@]5(F)CCOC5)CC4)nc(C(F)(F)F)n3)[C@@H]2C)CC1] | 1-(4-((2R,3R)-1-(6-(4- (2-((3S)-3- fluorotetrahydro-3- furanyl)-4-methyl-3- pyridinyl)-1- piperidinyl)-2- (trifluoromethyl)-4- pyrimidinyl)-2-methyl- 3-azetidinyl)-1- piperazinyl)-2-propen-1- one | E-1 | 618.3 |
| 2-282 | 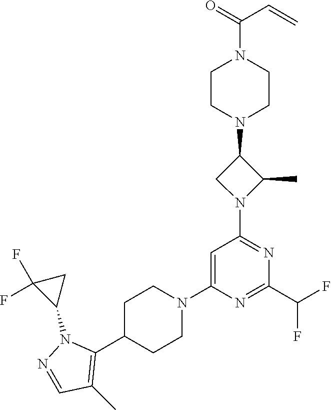 [SMILES: C=CC(=O)N1CCN([C@@H]2CN(c3cc(N4CCC(c5c(C)cnn5[C@H]5CC5(F)F)CC4)nc(C(F)F)n3)[C@@H]2C)CC1] | 1-(4-((2R,3R)-1-(6-(4- (1-((1S)-2,2- difluorocyclopropyl)-4- methyl-1H-pyrazol-5- yl)-1-piperidinyl)-2- (difluoromethyl)-4- pyrimidinyl)-2-methyl- 3-azetidinyl)-1- piperazinyl)-2-propen-1- one | E-2 | 577.2 |
| 2-283 | 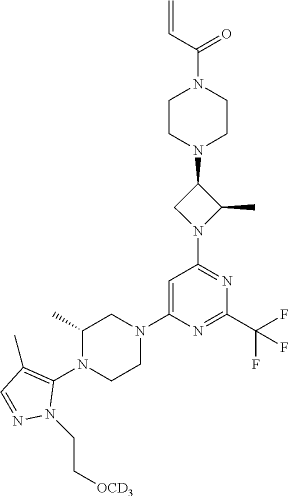 | 1-(4-((2R,3R)-2-methyl- 1-(6-((3R)-3-methyl-4- (4-methyl-1-(2- ((~2~H_3_)methyloxy) ethyl)-1H-pyrazol-5-yl)- 1-piperazinyl)-2- (trifluoromethyl)-4- pyrimidinyl)-3- azetidinyl)-1- piperazinyl)-2-propen-1- one | E-1 | 595.0 |
| 2-284 | 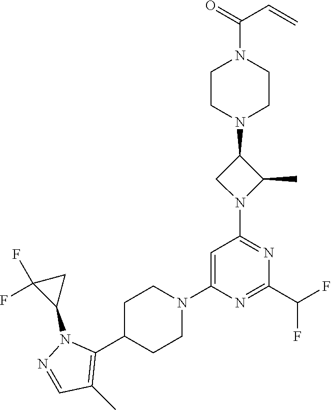 [SMILES: C=CC(=O)N1CCN([C@@H]2CN(c3cc(N4CCC(c5c(C)cnn5[C@@H]5CC5(F)F)CC4)nc(C(F)F)n3)[C@@H]2C)CC1] | 1-(4-((2R,3R)-1-(6-(4- (1-((1R)-2,2- difluorocyclopropyl)-4- methyl-1H-pyrazol-5- yl)-1-piperidinyl)-2- (difluoromethyl)-4- pyrimidinyl)-2-methyl- 3-azetidinyl)-1- piperazinyl)-2-propen-1- one | E-1 | 577.2 |
| 2-285 | 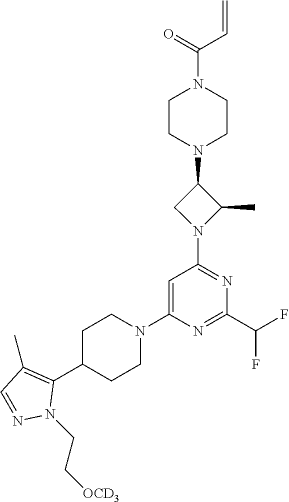 | 1-(4-((2R,3R)-1-(2- (difluoromethyl)-6-(4- (1-(2-(methoxy- d3)ethyl)-4-methyl-1H- pyrazol-5-yl)piperidin- 1-yl)pyrimidin-4-yl)-2- methylazetidin-3- yl)piperazin-1-yl)prop- 2-en-1-one | E-9 | 562.2 |
| 2-286 | 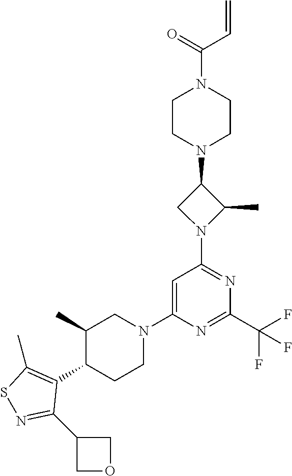 [SMILES: C=CC(=O)N1CCN([C@@H]2CN(c3cc(N4CC[C@H](c5c(C6COC6)nsc5C)[C@@H](C)C4)nc(C(F)(F)F)n3)[C@@H]2C)CC1] | 1-(4-((2R,3R)-2-methyl- 1-(6-((3R,4S)-3-methyl- 4-(5-methyl-3-(3- oxetanyl)-1,2-thiazol-4- yl)-1-piperidinyl)-2- (trifluoromethyl)-4- pyrimidinyl)-3- azetidinyl)-1- piperazinyl)-2-propen-1- one | E-1 | 605.8 |
| 2-287 | 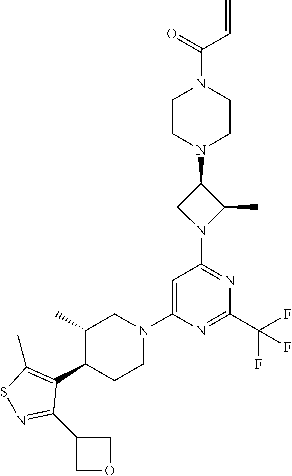 [SMILES: C=CC(=O)N1CCN([C@@H]2CN(c3cc(N4CC[C@@H](c5c(C6COC6)nsc5C)[C@H](C)C4)nc(C(F)(F)F)n3)[C@@H]2C)CC1] | 1-(4-((2R,3R)-2-methyl- 1-(6-((3S,4R)-3-methyl- 4-(5-methyl-3-(3- oxetanyl)-1,2-thiazol-4- yl)-1-piperidinyl)-2- (trifluoromethyl)-4- pyrimidinyl)-3- azetidinyl)-1- piperazinyl)-2-propen-1- one | E-1 | 606.0 |
| 2-288 | 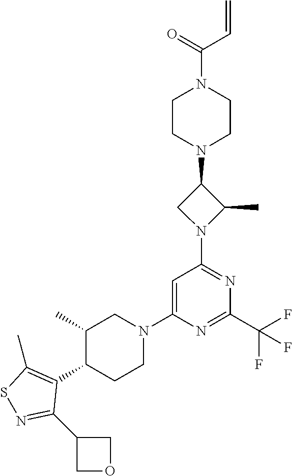 [SMILES: C=CC(=O)N1CCN([C@@H]2CN(c3cc(N4CC[C@H](c5c(C6COC6)nsc5C)[C@H](C)C4)nc(C(F)(F)F)n3)[C@@H]2C)CC1] | 1-(4-((2R,3R)-2-methyl- 1-(6-((3S,4S)-3-methyl- 4-(5-methyl-3-(3- oxetanyl)-1,2-thiazol-4- yl)-1-piperidinyl)-2- (trifluoromethyl)-4- pyrimidinyl)-3- azetidinyl)-1- piperazinyl)-2-propen-1- one | E-1 | 606.0 |
| 2-289 | 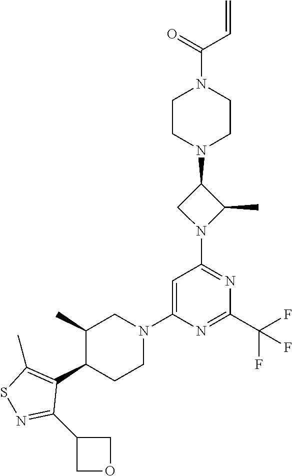 [SMILES: C=CC(=O)N1CCN([C@@H]2CN(c3cc(N4CC[C@@H](c5c(C6COC6)nsc5C)[C@@H](C)C4)nc(C(F)(F)F)n3)[C@@H]2C)CC1] | 1-(4-((2R,3R)-2-methyl- 1-(6-((3R,4R)-3-methyl- 4-(5-methyl-3-(3- oxetanyl)-1,2-thiazol-4- yl)-1-piperidinyl)-2- (trifluoromethyl)-4- pyrimidinyl)-3- azetidinyl)-1- piperazinyl)-2-propen-1- one | E-1 | 606.0 |
| 2-290 | 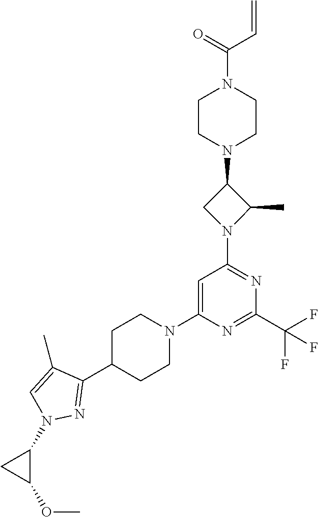 [SMILES: C=CC(=O)N1CCN([C@@H]2CN(c3cc(N4CCC(c5nn([C@H]6C[C@H]6OC)cc5C)CC4)nc(C(F)(F)F)n3)[C@@H]2C)CC1] | 1-(4-((2R,3R)-1-(6-(4- (1-((1S,2R)-2- methoxycyclopropyl)-4- methyl-1H-pyrazol-3- yl)-1-piperidinyl)-2- (trifluoromethyl)-4- pyrimidinyl)-2-methyl- 3-azetidinyl)-1- piperazinyl)-2-propen-1- one | E-1 | 589.0 |
| 2-291 | 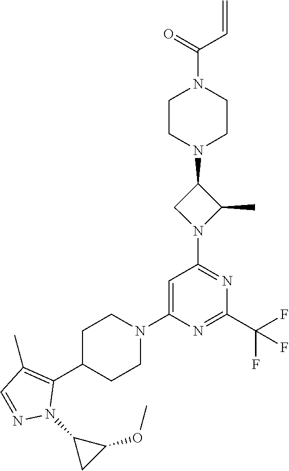 [SMILES: C=CC(=O)N1CCN([C@@H]2CN(c3cc(N4CCC(c5c(C)cnn5[C@H]5C[C@H]5OC)CC4)nc(C(F)(F)F)n3)[C@@H]2C)CC1] | 1-(4-((2R,3R)-1-(6-(4- (1-((1S,2R)-2- methoxycyclopropyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 589.0 |
| 2-292 | 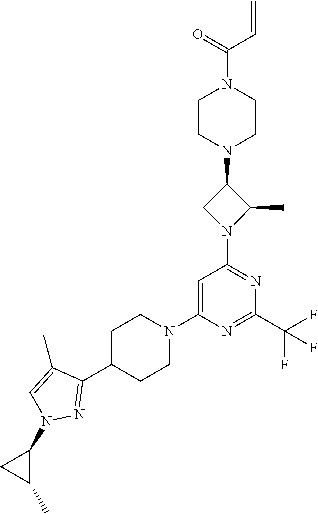 [SMILES: C=CC(=O)N1CCN([C@@H]2CN(c3cc(N4CCC(c5nn([C@@H]6C[C@H]6C)cc5C)CC4)nc(C(F)(F)F)n3)[C@@H]2C)CC1] | 1-(4-((2R,3R)-2-methyl- 1-(6-(4-(4-methyl-1- ((1R,2R)-2- methylcyclopropyl)-1H- pyrazol-3-yl)-1- piperidinyl)-2- (trifluoromethyl)-4- pyrimidinyl)-3- azetidinyl)-1- piperazinyl)-2-propen-1- one | E-1 | 573.0 |
| 2-293 | 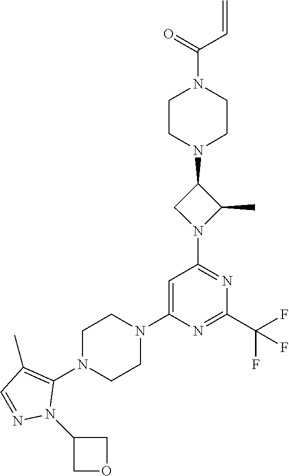 [SMILES: C=CC(=O)N1CCN([C@@H]2CN(c3cc(N4CCN(c5c(C)cnn5C5COC5)CC4)nc(C(F)(F)F)n3)[C@@H]2C)CC1] | 1-(4-((2R,3R)-2-methyl- 1-(6-(4-(4-methyl-1-(3- oxetanyl)-1H-pyrazol-5- yl)-1-piperazinyl)-2- (trifluoromethyl)-4- pyrimidinyl)-3- azetidinyl)-1- piperazinyl)-2-propen-1- one | E-1 | 576.1 |
| 2-294 | 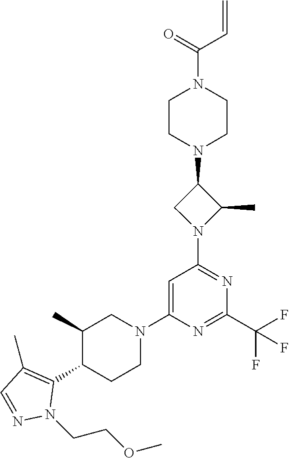 [SMILES: C=CC(=O)N1CCN([C@@H]2CN(c3cc(N4CC[C@H](c5c(C)cnn5CCOC)[C@@H](C)C4)nc(C(F)(F)F)n3)[C@@H]2C)CC1] | 1-(4-((2R,3R)-1-(6- ((3R,4S)-4-(1-(2- methoxyethyl)-4- methyl-1H-pyrazol-5- yl)-3-methyl-1- piperidinyl)-2- (trifluoromethyl)-4- pyrimidinyl)-2-methyl- 3-azetidinyl)-1- piperazinyl)-2-propen-1- one | E-1 | 591.0 |
| 2-295 | 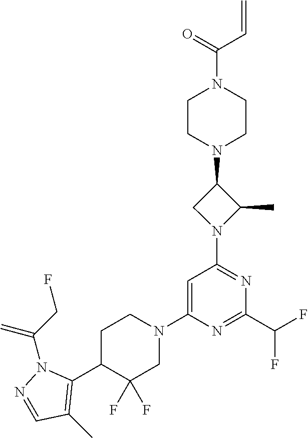 [SMILES: C=CC(=O)N1CCN([C@@H]2CN(c3cc(N4CCC(c5c(C)cnn5C(=C)CF)C(F)(F)C4)nc(C(F)F)n3)[C@@H]2C)CC1] | 1-(4-((2R,3R)-1-(6-(3,3- difluoro-4-(1-(3-fluoro- 1-propen-2-yl)-4- methyl-1H-pyrazol-5- yl)-1-piperidinyl)-2- (difluoromethyl)-4- pyrimidinyl)-2-methyl- 3-azetidinyl)-1- piperazinyl)-2-propen-1- one | E-2 | 595.0 |
| 2-296 | 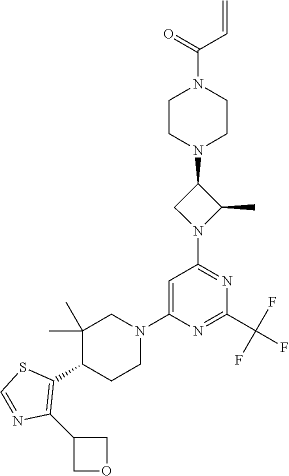 [SMILES: C=CC(=O)N1CCN([C@@H]2CN(c3cc(N4CC[C@H](c5scnc5C5COC5)C(C)(C)C4)nc(C(F)(F)F)n3)[C@@H]2C)CC1] | 1-(4-((2R,3R)-1-(6- ((4S)-3,3-dimethyl-4-(4- (3-oxetanyl)-1,3-thiazol- 5-yl)-1-piperidinyl)-2- (trifluoromethyl)-4- pyrimidinyl)-2-methyl- 3-azetidinyl)-1- piperazinyl)-2-propen-1- one | E-1 | 606.0 |
| 2-297 | 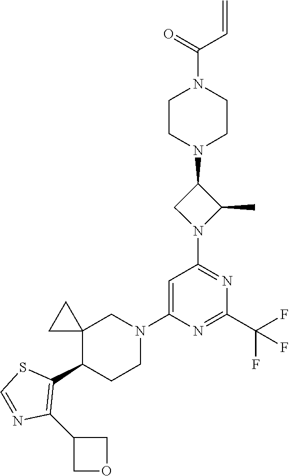 [SMILES: C=CC(=O)N1CCN([C@@H]2CN(c3cc(N4CC[C@@H](c5scnc5C5COC5)C5(CC5)C4)nc(C(F)(F)F)n3)[C@@H]2C)CC1] | 1-(4-((2R,3R)-2-methyl- 1-(6-((8R)-8-(4-(3- oxetanyl)-1,3-thiazol-5- yl)-5- azaspiro[2.5]octan-5- yl)-2-(trifluoromethyl)- 4-pyrimidinyl)-3- azetidinyl)-1- piperazinyl)-2-propen-1- one | E-1 | 604.2 |
| 2-298 | 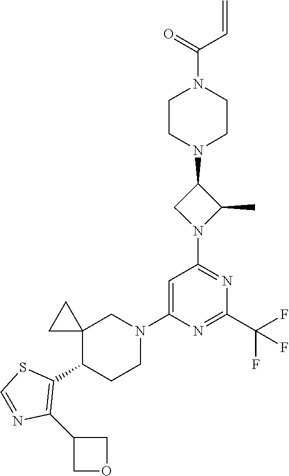 [SMILES: C=CC(=O)N1CCN([C@@H]2CN(c3cc(N4CC[C@H](c5scnc5C5COC5)C5(CC5)C4)nc(C(F)(F)F)n3)[C@@H]2C)CC1] | 1-(4-((2R,3R)-2-methyl- 1-(6-((8S)-8-(4-(3- oxetanyl)-1,3-thiazol-5- yl)-5- azaspiro[2.5]octan-5- yl)-2-(trifluoromethyl)- 4-pyrimidinyl)-3- azetidinyl)-1- piperazinyl)-2-propen-1- one | E-1 | 604.3 |
| 2-299 | 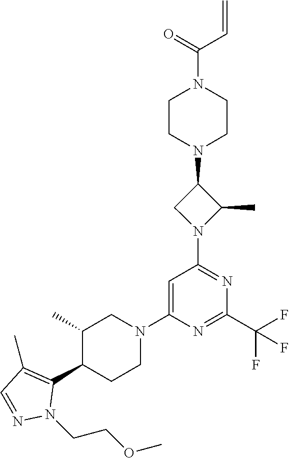 [SMILES: C=CC(=O)N1CCN([C@@H]2CN(c3cc(N4CC[C@@H](c5c(C)cnn5CCOC)[C@H](C)C4)nc(C(F)(F)F)n3)[C@@H]2C)CC1] | 1-(4-((2R,3R)-1-(6- ((3S,4R)-4-(1-(2- methoxyethyl)-4- methyl-1H-pyrazol-5- yl)-3-methyl-1- piperidinyl)-2- (trifluoromethyl)-4- pyrimidinyl)-2-methyl- 3-azetidinyl)-1- piperazinyl)-2-propen-1- one | E-1 | 591.0 |
| 2-300 | 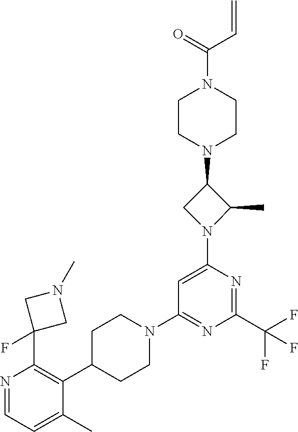 [SMILES: C=CC(=O)N1CCN([C@@H]2CN(c3cc(N4CCC(c5c(C)ccnc5C5(F)CN(C)C5)CC4)nc(C(F)(F)F)n3)[C@@H]2C)CC1] | 1-(4-((2R,3R)-1-(6-(4- (2-(3-fluoro-1-methyl-3- azetidinyl)-4-methyl-3- pyridinyl)-1- piperidinyl)-2- (trifluoromethyl)-4- pyrimidinyl)-2-methyl- 3-azetidinyl)-1- piperazinyl)-2-propen-1- one | E-1 | 617.3 |
| 2-301 | 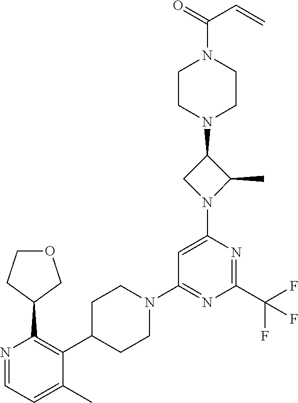 [SMILES: C=CC(=O)N1CCN([C@@H]2CN(c3cc(N4CCC(c5c(C)ccnc5[C@H]5CCOC5)CC4)nc(C(F)(F)F)n3)[C@@H]2C)CC1] | 1-(4-((2R,3R)-2-methyl- 1-(6-(4-(4-methyl-2- ((3R)-tetrahydro-3- furanyl)-3-pyridinyl)-1- piperidinyl)-2- (trifluoromethyl)-4- pyrimidinyl)-3- azetidinyl)-1- piperazinyl)-2-propen-1- one | E-8 | 600.2 |
| 2-302 | 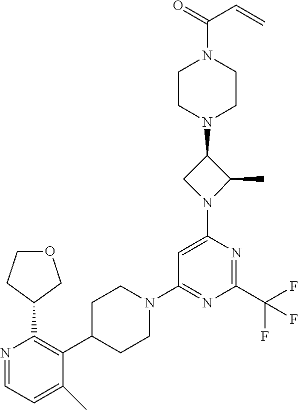 [SMILES: C=CC(=O)N1CCN([C@@H]2CN(c3cc(N4CCC(c5c(C)ccnc5[C@@H]5CCOC5)CC4)nc(C(F)(F)F)n3)[C@@H]2C)CC1] | 1-(4-((2R,3R)-2-methyl- 1-(6-(4-(4-methyl-2- ((3S)-tetrahydro-3- furanyl)-3-pyridinyl)-1- piperidinyl)-2- (trifluoromethyl)-4- pyrimidinyl)-3- azetidinyl)-1- piperazinyl)-2-propen-1- one | E-8 | 600.2 |
| 2-303 | 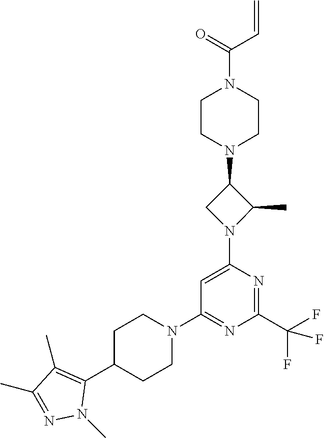 [SMILES: C=CC(=O)N1CCN([C@@H]2CN(c3cc(N4CCC(c5c(C)c(C)nn5C)CC4)nc(C(F)(F)F)n3)[C@@H]2C)CC1] | 1-(4-((2R,3R)-2-methyl- 1-(2-(trifluoromethyl)-6- (4-(1,3,4-trimethyl-1H- pyrazol-5-yl)-1- piperidinyl)-4- pyrimidinyl)-3- azetidinyl)-1- piperazinyl)-2-propen-1- one | E-1 | 547.3 |
| 2-304 | 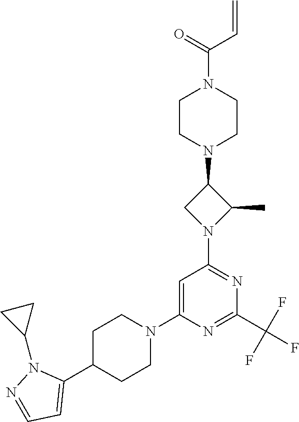 [SMILES: C=CC(=O)N1CCN([C@@H]2CN(c3cc(N4CCC(c5ccnn5C5CC5)CC4)nc(C(F)(F)F)n3)[C@@H]2C)CC1] | 1-(4-((2R,3R)-1-(6-(4- (1-cyclopropyl-1H- pyrazol-5-yl)-1- piperidinyl)-2- (trifluoromethyl)-4- pyrimidinyl)-2-methyl- 3-azetidinyl)-1- piperazinyl)-2-propen-1- one | E-1 | 545.1 |
| 2-305 | 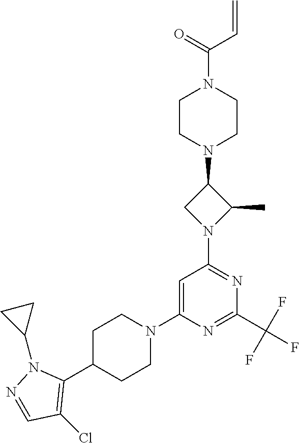 [SMILES: C=CC(=O)N1CCN([C@@H]2CN(c3cc(N4CCC(c5c(Cl)cnn5C5CC5)CC4)nc(C(F)(F)F)n3)[C@@H]2C)CC1] | 1-(4-((2R,3R)-1-(6-(4- (4-chloro-1-cyclopropyl- 1H-pyrazol-5-yl)-1- piperidinyl)-2- (trifluoromethyl)-4- pyrimidinyl)-2-methyl- 3-azetidinyl)-1- piperazinyl)-2-propen-1- one | E-1 | 579.1 |
| 2-310 | 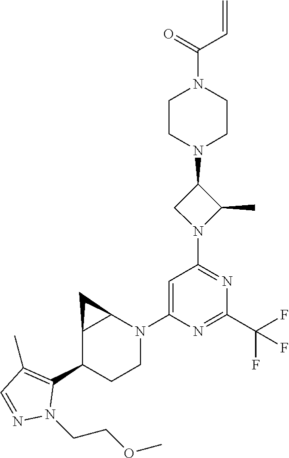 [SMILES: C=CC(=O)N1CCN([C@@H]2CN(c3cc(N4CC[C@@H](c5c(C)cnn5CCOC)[C@H]5C[C@H]54)nc(C(F)(F)F)n3)[C@@H]2C)CC1] | 1-(4-((2R,3R)-1-(6- ((1R,5R,6R)-5-(1-(2- methoxyethyl)-4- methyl-1H-pyrazol-5- yl)-2- azabicyclo[4.1.0]heptan- 2-yl)-2- (trifluoromethyl)-4- pyrimidinyl)-2-methyl- 3-azetidinyl)-1- piperazinyl)-2-propen-1- one | E-1 | 589.0 |
| 2-311 | 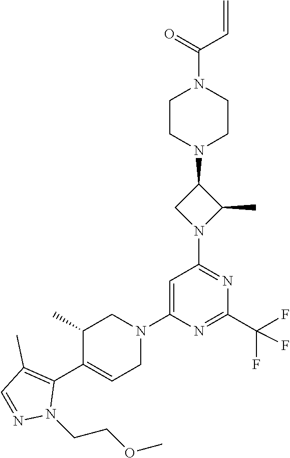 [SMILES: C=CC(=O)N1CCN([C@@H]2CN(c3cc(N4CC=C(c5c(C)cnn5CCOC)[C@H](C)C4)nc(C(F)(F)F)n3)[C@@H]2C)CC1] | 1-(4-((2R,3R)-1-(6- ((3S)-4-(1-(2- methoxyethyl)-4- methyl-1H-pyrazol-5- yl)-3-methyl-3,6- dihydro-1(2H)- pyridinyl)-2- (trifluoromethyl)-4- pyrimidinyl)-2-methyl- 3-azetidinyl)-1- piperazinyl)-2-propen-1- one | E-1 | 589.0 |
| 2-312 | 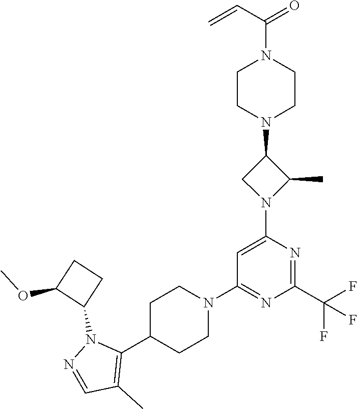 [SMILES: C=CC(=O)N1CCN([C@@H]2CN(c3cc(N4CCC(c5c(C)cnn5[C@H]5CC[C@@H]5OC)CC4)nc(C(F)(F)F)n3)[C@@H]2C)CC1] | 1-(4-((2R,3R)-1-(6-(4- (1-((1S,2S)-2- methoxycyclobutyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 603.2 |
| 2-313 | 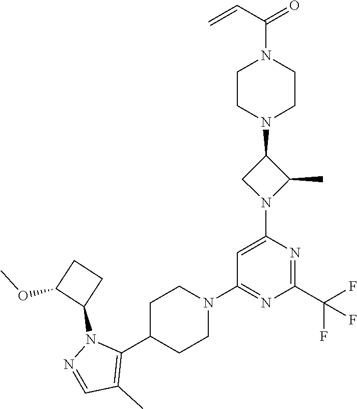 [SMILES: C=CC(=O)N1CCN([C@@H]2CN(c3cc(N4CCC(c5c(C)cnn5[C@@H]5CC[C@H]5OC)CC4)nc(C(F)(F)F)n3)[C@@H]2C)CC1] | 1-(4-((2R,3R)-1-(6-(4- (1-((1R,2R)-2- methoxycyclobutyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 603.2 |
| 2-314 | 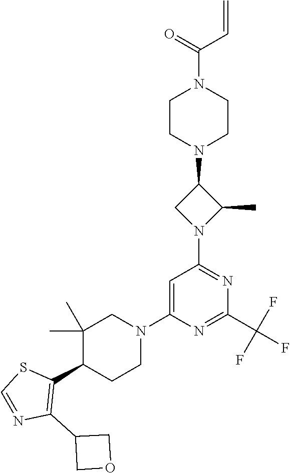 [SMILES: C=CC(=O)N1CCN([C@@H]2CN(c3cc(N4CC[C@@H](c5scnc5C5COC5)C(C)(C)C4)nc(C(F)(F)F)n3)[C@@H]2C)CC1] | 1-(4-((2R,3R)-1-(6- ((4R)-3,3-dimethyl-4-(4- (3-oxetanyl)-1,3-thiazol- 5-yl)-1-piperidinyl)-2- (trifluoromethyl)-4- pyrimidinyl)-2-methyl- 3-azetidinyl)-1- piperazinyl)-2-propen-1- one | E-1 | 606.3 |
| 2-315 | 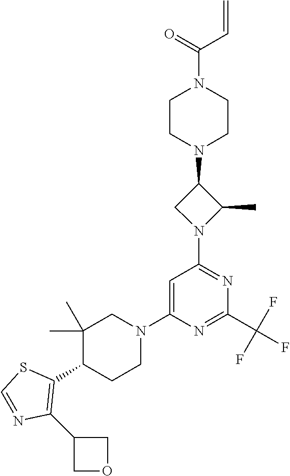 [SMILES: C=CC(=O)N1CCN([C@@H]2CN(c3cc(N4CC[C@H](c5scnc5C5COC5)C(C)(C)C4)nc(C(F)(F)F)n3)[C@@H]2C)CC1] | 1-(4-((2R,3R)-1-(6- ((4S)-3,3-dimethyl-4-(4- (3-oxetanyl)-1,3-thiazol- 5-yl)-1-piperidinyl)-2- (trifluoromethyl)-4- pyrimidinyl)-2-methyl- 3-azetidinyl)-1- piperazinyl)-2-propen-1- one | E-1 | 606.3 |
| 2-316 | 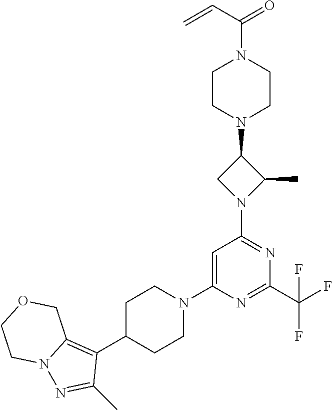 [SMILES: C=CC(=O)N1CCN([C@@H]2CN(c3cc(N4CCC(c5c(C)nn6c5COCC6)CC4)nc(C(F)(F)F)n3)[C@@H]2C)CC1] | 1-(4-((2R,3R)-2-methyl- 1-(6-(4-(2-methyl-6,7- dihydro-4H- pyrazolo[5,1- c][1,4]oxazin-3-yl)-1- piperidinyl)-2- (trifluoromethyl)-4- pyrimidinyl)-3- azetidinyl)-1- piperazinyl)-2-propen-1- one | E-8 | 575.1 |
| 2-317 | 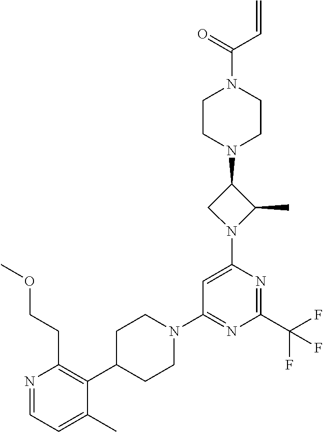 [SMILES: C=CC(=O)N1CCN([C@@H]2CN(c3cc(N4CCC(c5c(C)ccnc5CCOC)CC4)nc(C(F)(F)F)n3)[C@@H]2C)CC1] | 1-(4-((2R,3R)-1-(6-(4- (2-(2-methoxyethyl)-4- methyl-3-pyridinyl)-1- piperidinyl)-2- (trifluoromethyl)-4- pyrimidinyl)-2-methyl- 3-azetidinyl)-1- piperazinyl)-2-propen-1- one | E-8 | 588.3 |
| 2-318 | 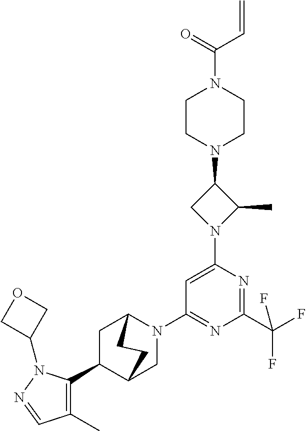 [SMILES: C=CC(=O)N1CCN([C@@H]2CN(c3cc(N4C[C@H]5CC[C@@H]4C[C@@H]5c4c(C)cnn4C4COC4)nc(C(F)(F)F)n3)[C@@H]2C)CC1] | 1-(4-((2R,3R)-2-methyl- 1-(6-((1R,4S,5S)-5-(4- methyl-1-(3-oxetanyl)- 1H-pyrazol-5-yl)-2- azabicyclo[2.2.2]octan- 2-yl)-2- (trifluoromethyl)-4- pyrimidinyl)-3- azetidinyl)-1- piperazinyl)-2-propen-1- one | E-8 | 600.6 |
| 2-319 | 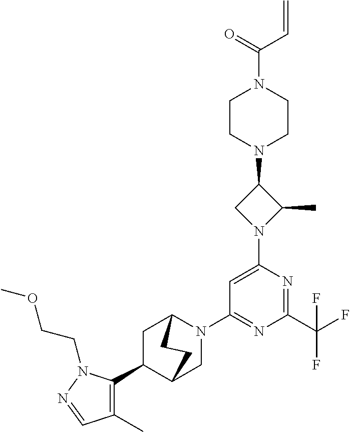 [SMILES: C=CC(=O)N1CCN([C@@H]2CN(c3cc(N4C[C@H]5CC[C@@H]4C[C@@H]5c4c(C)cnn4CCOC)nc(C(F)(F)F)n3)[C@@H]2C)CC1] | 1-(4-((2R,3R)-1-(6- ((1R,4S,5S)-5-(1-(2- methoxyethyl)-4- methyl-1H-pyrazol-5- yl)-2- azabicyclo[2.2.2]octan- 2-yl)-2- (trifluoromethyl)-4- pyrimidinyl)-2-methyl- 3-azetidinyl)-1- piperazinyl)-2-propen-1- one | E-8 | 602.7 |
| 2-320 | 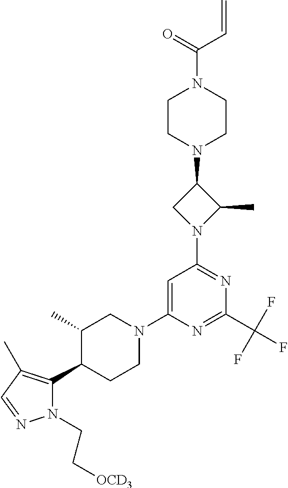 | 1-(4-((2R,3R)-1-(6- ((3S,4R)-4-(1-(2- (methoxy-d3)ethyl)-4- methyl-1H-pyrazol-5- yl)-3-methylpiperidin-1- yl)-2- (trifluoromethyl)pyrimidin- 4-yl)-2- methylazetidin-3- yl)piperazin-1-yl)prop- 2-en-1-one | E-8 | 594.1 |
| 2-321 | 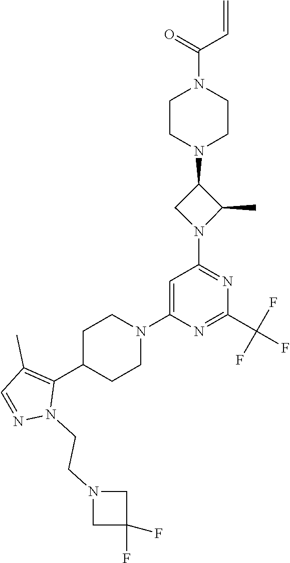 [SMILES: C=CC(=O)N1CCN([C@@H]2CN(c3cc(N4CCC(c5c(C)cnn5CCN5CC(F)(F)C5)CC4)nc(C(F)(F)F)n3)[C@@H]2C)CC1] | 1-(4-((2R,3R)-1-(6-(4- (1-(2-(3,3-difluoro-1- azetidinyl)ethyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-8 | 638.1 |
| 2-322 | 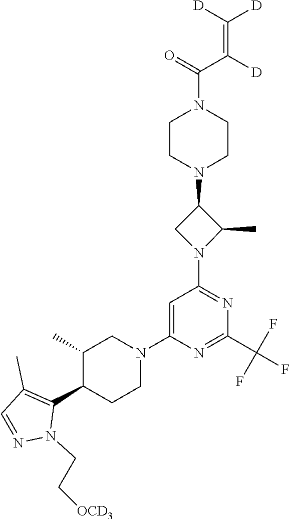 | 1-(4-((2R,3R)-1-(6- ((3S,4R)-4-(1-(2- (methoxy-d3)ethyl)-4- methyl-1H-pyrazol-5- yl)-3-methylpiperidin-1- yl)-2- (trifluoromethyl)pyrimidin- 4-yl)-2- methylazetidin-3- yl)piperazin-1-yl)prop- 2-en-1-one-2,3,3-d3 | E-8 | 597.1 |
| 2-323 | 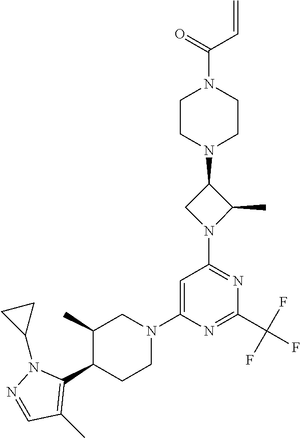 [SMILES: C=CC(=O)N1CCN([C@@H]2CN(c3cc(N4CC[C@@H](c5c(C)cnn5C5CC5)[C@@H](C)C4)nc(C(F)(F)F)n3)[C@@H]2C)CC1] | 1-(4-((2R,3R)-1-(6- ((3R,4R)-4-(1- cyclopropyl-4-methyl- 1H-pyrazol-5-yl)-3- methyl-1-piperidinyl)-2- (trifluoromethyl)-4- pyrimidinyl)-2-methyl- 3-azetidinyl)-1- piperazinyl)-2-propen-1- one | E-8 | 573.1 |
| 2-324 | 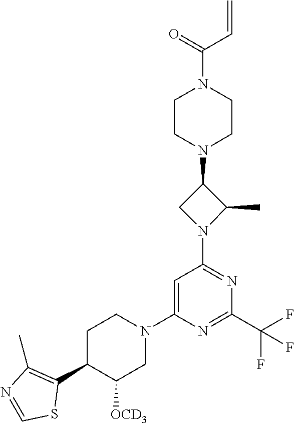 | 1-(4-((2R,3R)-1-(6- ((3R,4S)-3-(methoxy- d3)-4-(4-methylthiazol- 5-yl)piperidin-1-yl)-2- (trifluoromethyl)pyrimidin- 4-yl)-2- methylazetidin-3- yl)piperazin-1-yl)prop- 2-en-1-one | E-8 | 569.0 |
| | mixture of trans isomers at piperidine | | | |
| 2-325 | 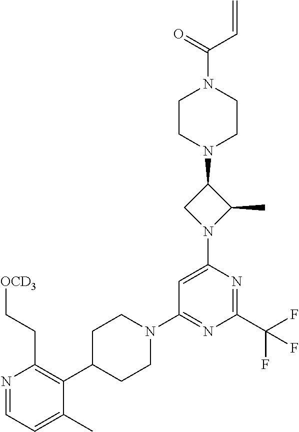 | 1-(4-((2R,3R)-1-(6-(4- (2-(2-(methoxy- d3)ethyl)-4- methylpyridin-3- yl)piperidin-1-yl)-2- (trifluoromethyl)pyrimidin- 4-yl)-2- methylazetidin-3- yl)piperazin-1-yl)prop- 2-en-1-one | E-8 | 591.3 |
| 2-326 | 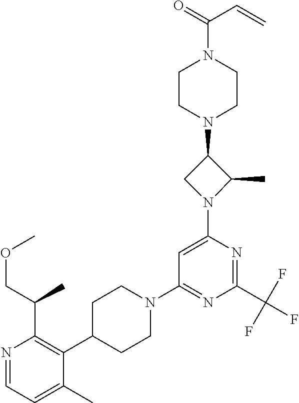 [SMILES: C=CC(=O)N1CCN([C@@H]2CN(c3cc(N4CCC(c5c(C)ccnc5[C@H](C)COC)CC4)nc(C(F)(F)F)n3)[C@@H]2C)CC1] | 1-(4-((2R,3R)-1-(6-(4- (2-((2S)-1-methoxy-2- propanyl)-4-methyl-3- pyridinyl)-1- piperidinyl)-2- (trifluoromethyl)-4- pyrimidinyl)-2-methyl- 3-azetidinyl)-1- piperazinyl)-2-propen-1- one | E-8 | 602.1 |
| 2-327 | 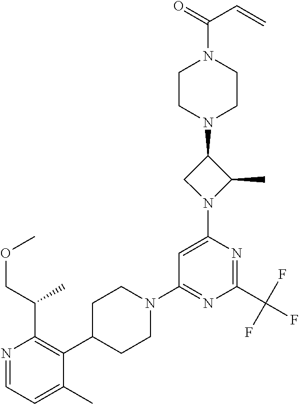 [SMILES: C=CC(=O)N1CCN([C@@H]2CN(c3cc(N4CCC(c5c(C)ccnc5[C@@H](C)COC)CC4)nc(C(F)(F)F)n3)[C@@H]2C)CC1] | 1-(4-((2R,3R)-1-(6-(4- (2-((2R)-1-methoxy-2- propanyl)-4-methyl-3- pyridinyl)-1- piperidinyl)-2- (trifluoromethyl)-4- pyrimidinyl)-2-methyl- 3-azetidinyl)-1- piperazinyl)-2-propen-1- one | E-8 | 602.3 |
| 2-328 | 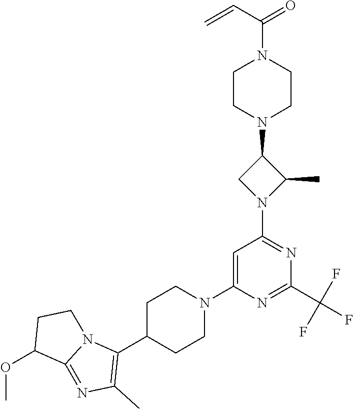 [SMILES: C=CC(=O)N1CCN([C@@H]2CN(c3cc(N4CCC(c5c(C)nc6n5CCC6OC)CC4)nc(C(F)(F)F)n3)[C@@H]2C)CC1] | 1-(4-((2R,3R)-1-(6-(4- (7-methoxy-2-methyl- 6,7-dihydro-5H- pyrrolo[1,2-a]imidazol- 3-yl)-1-piperidinyl)-2- (trifluoromethyl)-4- pyrimidinyl)-2-methyl- 3-azetidinyl)-1- piperazinyl)-2-propen-1- one | E-8 | 589.3 |
| 2-329 | 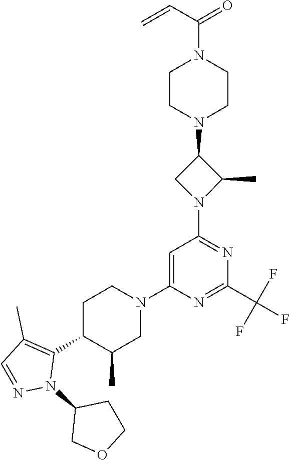 [SMILES: C=CC(=O)N1CCN([C@@H]2CN(c3cc(N4CC[C@@H](c5c(C)cnn5[C@H]5CCOC5)[C@H](C)C4)nc(C(F)(F)F)n3)[C@@H]2C)CC1] | 1-(4-((2R,3R)-2-methyl- 1-(6-((3S,4R)-3-methyl- 4-(4-methyl-1-((3S)- tetrahydro-3-furanyl)- 1H-pyrazol-5-yl)-1- piperidinyl)-2- (trifluoromethyl)-4- pyrimidinyl)-3- azetidinyl)-1- piperazinyl)-2-propen-1- one | E-1 | 603.2 |
| 2-330 | 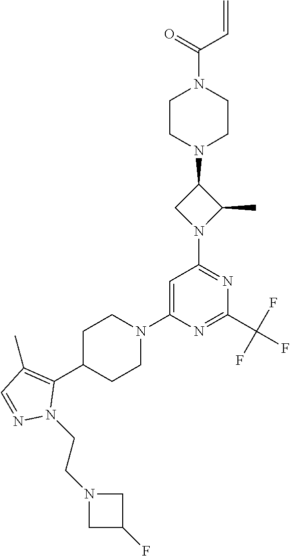 [SMILES: C=CC(=O)N1CCN([C@@H]2CN(c3cc(N4CCC(c5c(C)cnn5CCN5CC(F)C5)CC4)nc(C(F)(F)F)n3)[C@@H]2C)CC1] | 1-(4-((2R,3R)-1-(6-(4- (1-(2-(3-fluoro-1- azetidinyl)ethyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 620.1 |
| 2-331 | 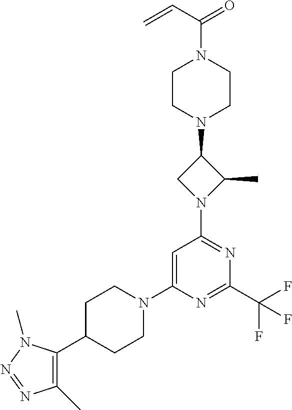 [SMILES: C=CC(=O)N1CCN([C@@H]2CN(c3cc(N4CCC(c5c(C)nnn5C)CC4)nc(C(F)(F)F)n3)[C@@H]2C)CC1] | 1-(4-((2R,3R)-1-(6-(4- (1,4-dimethyl-1H-1,2,3- triazol-5-yl)-1- piperidinyl)-2- (trifluoromethyl)-4- pyrimidinyl)-2-methyl- 3-azetidinyl)-1- piperazinyl)-2-propen-1- one | E-8 | 534.2 |
| 2-332 | 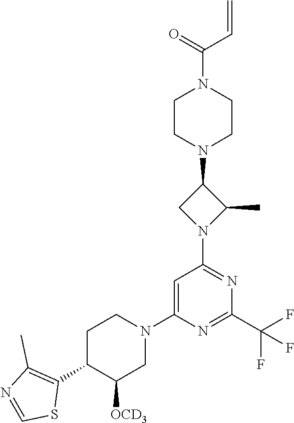 | 1-(4-((2R,3R)-1-(6- ((3S,4R)-3-(methoxy- d3)-4-(4-methylthiazol- 5-yl)piperidin-1-yl)-2- (trifluoromethyl)pyrimidin- 4-yl)-2- methylazetidin-3- yl)piperazin-1-yl)prop- 2-en-1-one | E-8 | 569.3 |
| 2-333 | 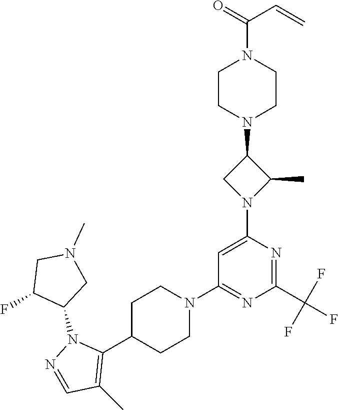 [SMILES: C=CC(=O)N1CCN([C@@H]2CN(c3cc(N4CCC(c5c(C)cnn5[C@H]5CN(C)C[C@H]5F)CC4)nc(C(F)(F)F)n3)[C@@H]2C)CC1] | 1-(4-((2R,3R)-1-(6-(4- (1-((3S,4R)-4-fluoro-1- methyl-3-pyrrolidinyl)- 4-methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 620.3 |
| 2-334 | 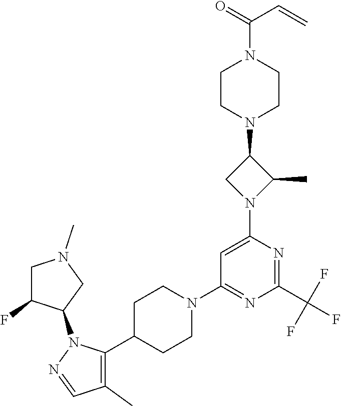 [SMILES: C=CC(=O)N1CCN([C@@H]2CN(c3cc(N4CCC(c5c(C)cnn5[C@@H]5CN(C)C[C@@H]5F)CC4)nc(C(F)(F)F)n3)[C@@H]2C)CC1] | 1-(4-((2R,3R)-1-(6-(4- (1-((3R,4S)-4-fluoro-1- methyl-3-pyrrolidinyl)- 4-methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 620.3 |
| 2-336 | 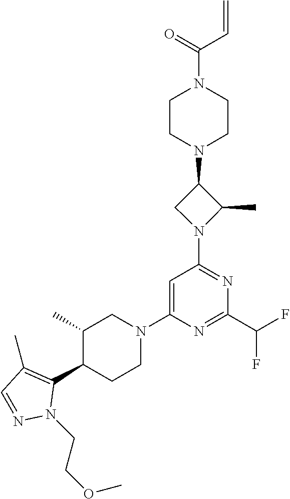 [SMILES: C=CC(=O)N1CCN([C@@H]2CN(c3cc(N4CC[C@@H](c5c(C)cnn5CCOC)[C@H](C)C4)nc(C(F)F)n3)[C@@H]2C)CC1] | 1-(4-((2R,3R)-1-(2- (difluoromethyl)-6- ((3S,4R)-4-(1-(2- methoxyethyl)-4- methyl-1H-pyrazol-5- yl)-3-methyl-1- piperidinyl)-4- pyrimidinyl)-2-methyl- 3-azetidinyl)-1- piperazinyl)-2-propen-1- one | E-2 | 573.1 |
| 2-338 | 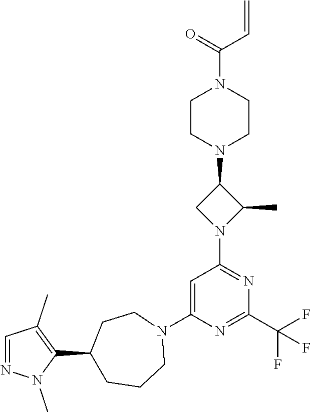 [SMILES: C=CC(=O)N1CCN([C@@H]2CN(c3cc(N4CCC[C@@H](c5c(C)cnn5C)CC4)nc(C(F)(F)F)n3)[C@@H]2C)CC1] | 1-(4-((2R,3R)-1-(6- ((4R)-4-(1,4-dimethyl- 1H-pyrazol-5-yl)-1- azepanyl)-2- (trifluoromethyl)-4- pyrimidinyl)-2-methyl- 3-azetidinyl)-1- piperazinyl)-2-propen-1- one | E-1 | 547.3 |
| 2-339 | 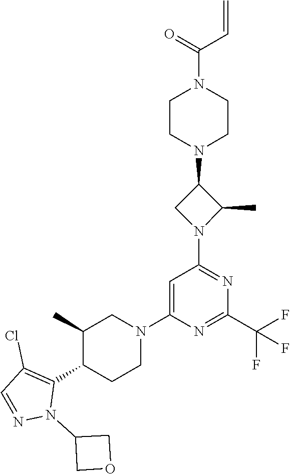 [SMILES: C=CC(=O)N1CCN([C@@H]2CN(c3cc(N4CC[C@H](c5c(Cl)cnn5C5COC5)[C@@H](C)C4)nc(C(F)(F)F)n3)[C@@H]2C)CC1] | 1-(4-((2R,3R)-1-(6- ((3R,4S)-4-(4-chloro-1- (3-oxetanyl)-1H- pyrazol-5-yl)-3-methyl- 1-piperidinyl)-2- (trifluoromethyl)-4- pyrimidinyl)-2-methyl- 3-azetidinyl)-1- piperazinyl)-2-propen-1- one | E-1 | 609.0 |
| 2-340 | 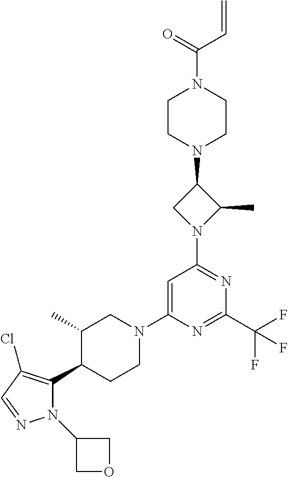 [SMILES: C=CC(=O)N1CCN([C@@H]2CN(c3cc(N4CC[C@@H](c5c(Cl)cnn5C5COC5)[C@H](C)C4)nc(C(F)(F)F)n3)[C@@H]2C)CC1] | 1-(4-((2R,3R)-1-(6- ((3S,4R)-4-(4-chloro-1- (3-oxetanyl)-1H- pyrazol-5-yl)-3-methyl- 1-piperidinyl)-2- (trifluoromethyl)-4- pyrimidinyl)-2-methyl- 3-azetidinyl)-1- piperazinyl)-2-propen-1- one | E-1 | 609.0 |
| 2-341 | 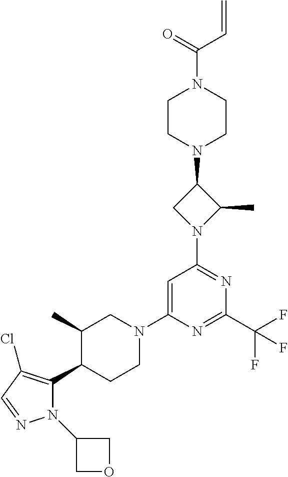 [SMILES: C=CC(=O)N1CCN([C@@H]2CN(c3cc(N4CC[C@@H](c5c(Cl)cnn5C5COC5)[C@@H](C)C4)nc(C(F)(F)F)n3)[C@@H]2C)CC1] | 1-(4-((2R,3R)-1-(6- ((3R,4R)-4-(4-chloro-1- (3-oxetanyl)-1H- pyrazol-5-yl)-3-methyl- 1-piperidinyl)-2- (trifluoromethyl)-4- pyrimidinyl)-2-methyl- 3-azetidinyl)-1- piperazinyl)-2-propen-1- one | E-1 | 608.8 |
| 2-342 | 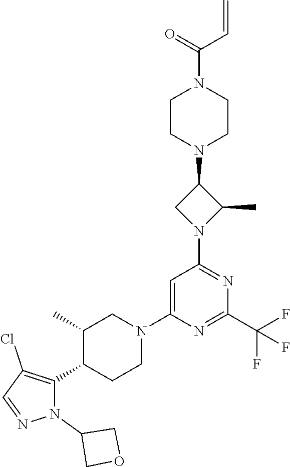 [SMILES: C=CC(=O)N1CCN([C@@H]2CN(c3cc(N4CC[C@H](c5c(Cl)cnn5C5COC5)[C@H](C)C4)nc(C(F)(F)F)n3)[C@@H]2C)CC1] | 1-(4-((2R,3R)-1-(6- ((3S,4S)-4-(4-chloro-1- (3-oxetanyl)-1H- pyrazol-5-yl)-3-methyl- 1-piperidinyl)-2- (trifluoromethyl)-4- pyrimidinyl)-2-methyl- 3-azetidinyl)-1- piperazinyl)-2-propen-1- one | E-1 | 608.8 |
| 2-343 | 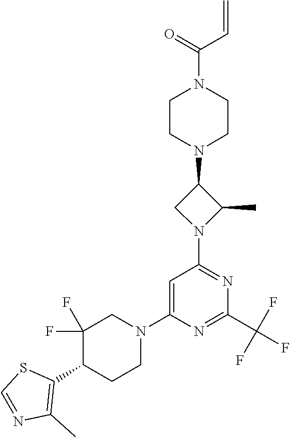 [SMILES: C=CC(=O)N1CCN([C@@H]2CN(c3cc(N4CC[C@H](c5scnc5C)C(F)(F)C4)nc(C(F)(F)F)n3)[C@@H]2C)CC1] | 1-(4-((2R,3R)-1-(6- ((4S)-3,3-difluoro-4-(4- methyl-1,3-thiazol-5- yl)-1-piperidinyl)-2- (trifluoromethyl)-4- pyrimidinyl)-2-methyl- 3-azetidinyl)-1- piperazinyl)-2-propen-1- one | E-1 | 572.2 |
| 2-344 | 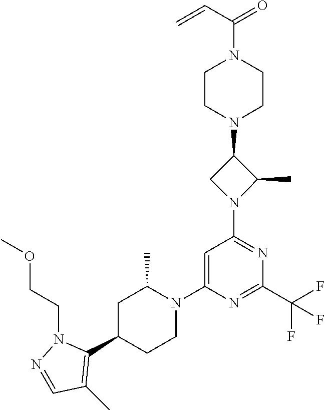 [SMILES: C=CC(=O)N1CCN([C@@H]2CN(c3cc(N4CC[C@@H](c5c(C)cnn5CCOC)C[C@@H]4C)nc(C(F)(F)F)n3)[C@@H]2C)CC1] | 1-(4-((2R,3R)-1-(6- ((2S,4R)-4-(1-(2- methoxyethyl)-4- methyl-1H-pyrazol-5- yl)-2-methyl-1- piperidinyl)-2- (trifluoromethyl)-4- pyrimidinyl)-2-methyl- 3-azetidinyl)-1- piperazinyl)-2-propen-1- one | E-1 | 591.2 |
| 2-346 | 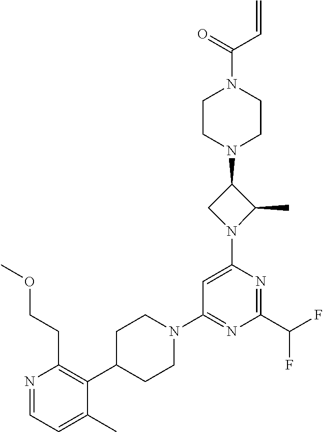 [SMILES: C=CC(=O)N1CCN([C@@H]2CN(c3cc(N4CCC(c5c(C)ccnc5CCOC)CC4)nc(C(F)F)n3)[C@@H]2C)CC1] | 1-(4-((2R,3R)-1-(2- (difluoromethyl)-6-(4- (2-(2-methoxyethyl)-4- methyl-3-pyridinyl)-1- piperidinyl)-4- pyrimidinyl)-2-methyl- 3-azetidinyl)-1- piperazinyl)-2-propen-1- one | E-1 | 570.3 |
| 2-349 | 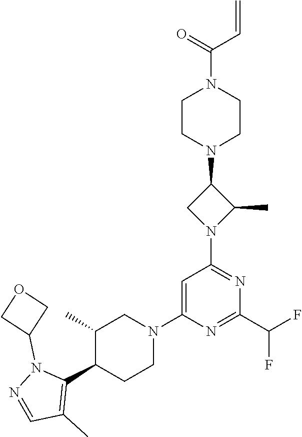 [SMILES: C=CC(=O)N1CCN([C@@H]2CN(c3cc(N4CC[C@@H](c5c(C)cnn5C5COC5)[C@H](C)C4)nc(C(F)F)n3)[C@@H]2C)CC1] | 1-(4-((2R,3R)-1-(2- (difluoromethyl)-6- ((3S,4R)-3-methyl-4-(4- methyl-1-(3-oxetanyl)- 1H-pyrazol-5-yl)-1- piperidinyl)-4- pyrimidinyl)-2-methyl- 3-azetidinyl)-1- piperazinyl)-2-propen-1- one | E-9 | 571.0 |
| 2-351 | 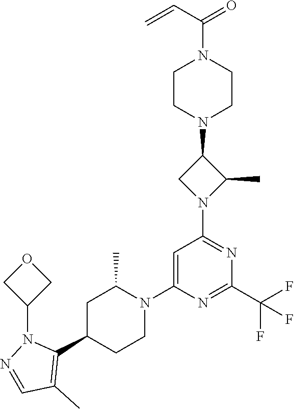 [SMILES: C=CC(=O)N1CCN([C@@H]2CN(c3cc(N4CC[C@@H](c5c(C)cnn5C5COC5)C[C@@H]4C)nc(C(F)(F)F)n3)[C@@H]2C)CC1] | 1-(4-((2R,3R)-2-methyl- 1-(6-((2S,4R)-2-methyl- 4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-1 | 589.3 |
| 2-352 | 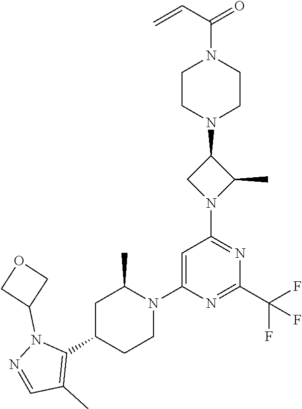 [SMILES: C=CC(=O)N1CCN([C@@H]2CN(c3cc(N4CC[C@H](c5c(C)cnn5C5COC5)C[C@H]4C)nc(C(F)(F)F)n3)[C@@H]2C)CC1] | 1-(4-((2R,3R)-2-methyl- 1-(6-((2R,4S)-2-methyl- 4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-1 | 589.3 |
| 2-354 | 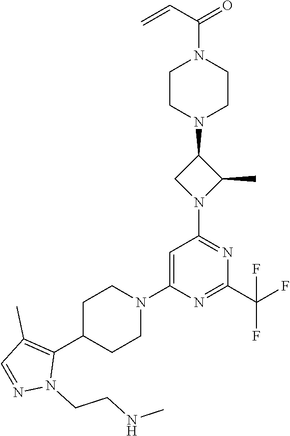 [SMILES: C=CC(=O)N1CCN([C@@H]2CN(c3cc(N4CCC(c5c(C)cnn5CCNC)CC4)nc(C(F)(F)F)n3)[C@@H]2C)CC1] | 1-(4-((2R,3R)-2-methyl- 1-(6-(4-(4-methyl-1-(2- (methylamino)ethyl)- 1H-pyrazol-5-yl)-1- piperidinyl)-2- (trifluoromethyl)-4- pyrimidinyl)-3- azetidinyl)-1- piperazinyl)-2-propen-1- one | E-1 | 576.3 |
| 2-355 | 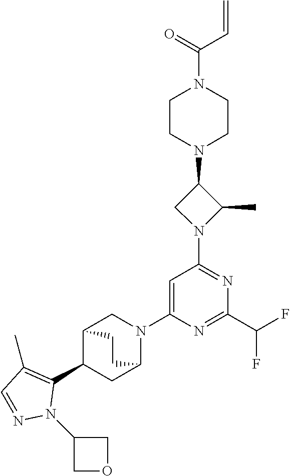 [SMILES: C=CC(=O)N1CCN([C@@H]2CN(c3cc(N4C[C@H]5CC[C@@H]4C[C@H]5c4c(C)cnn4C4COC4)nc(C(F)F)n3)[C@@H]2C)CC1] | 1-(4-((2R,3R)-1-(2- (difluoromethyl)-6- ((1R,4S,5R)-5-(4- methyl-1-(3-oxetanyl)- 1H-pyrazol-5-yl)-2- azabicyclo[2.2.2]octan- 2-yl)-4-pyrimidinyl)-2- methyl-3-azetidinyl)-1- piperazinyl)-2-propen-1- one | E-2 | 583.2 |
| 2-356 | 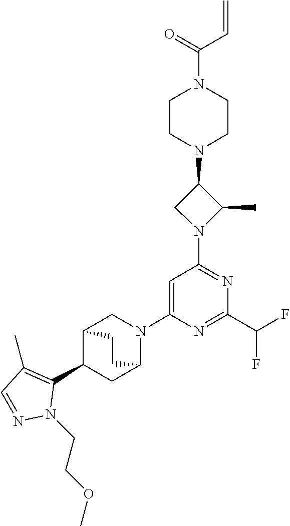 [SMILES: C=CC(=O)N1CCN([C@@H]2CN(c3cc(N4C[C@H]5CC[C@@H]4C[C@H]5c4c(C)cnn4CCOC)nc(C(F)F)n3)[C@@H]2C)CC1] | 1-(4-((2R,3R)-1-(2- (difluoromethyl)-6- ((1R,4S,5R)-5-(1-(2- methoxyethyl)-4- methyl-1H-pyrazol-5- yl)-2- azabicyclo[2.2.2]octan- 2-yl)-4-pyrimidinyl)-2- methyl-3-azetidinyl)-1- piperazinyl)-2-propen-1- one | E-2 | 585.4 |
| 2-358 | 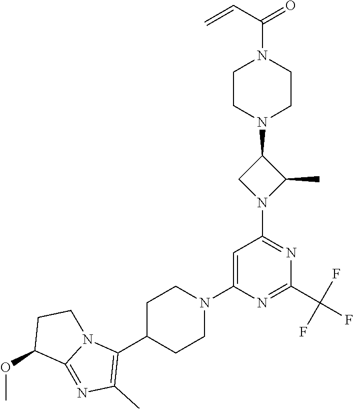 [SMILES: C=CC(=O)N1CCN([C@@H]2CN(c3cc(N4CCC(c5c(C)nc6n5CC[C@@H]6OC)CC4)nc(C(F)(F)F)n3)[C@@H]2C)CC1] | 1-(4-((2R,3R)-1-(6-(4- ((7S)-7-methoxy-2- methyl-6,7-dihydro-5H- pyrrolo[1,2-a]imidazol- 3-yl)-1-piperidinyl)-2- (trifluoromethyl)-4- pyrimidinyl)-2-methyl- 3-azetidinyl)-1- piperazinyl)-2-propen-1- one | E-1 | 589.2 |
| 2-359 | 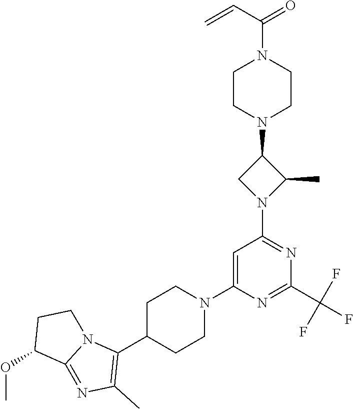 [SMILES: C=CC(=O)N1CCN([C@@H]2CN(c3cc(N4CCC(c5c(C)nc6n5CC[C@H]6OC)CC4)nc(C(F)(F)F)n3)[C@@H]2C)CC1] | 1-(4-((2R,3R)-1-(6-(4- ((7R)-7-methoxy-2- methyl-6,7-dihydro-5H- pyrrolo[1,2-a]imidazol- 3-yl)-1-piperidinyl)-2- (trifluoromethyl)-4- pyrimidinyl)-2-methyl- 3-azetidinyl)-1- piperazinyl)-2-propen-1- one | E-1 | 589.2 |
| 2-360 | 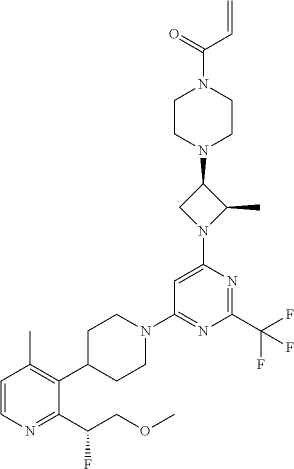 [SMILES: C=CC(=O)N1CCN([C@@H]2CN(c3cc(N4CCC(c5c(C)ccnc5[C@@H](F)COC)CC4)nc(C(F)(F)F)n3)[C@@H]2C)CC1] | 1-(4-((2R,3R)-1-(6-(4- (2-((1R)-1-fluoro-2- methoxyethyl)-4- methyl-3-pyridinyl)-1- piperidinyl)-2- (trifluoromethyl)-4- pyrimidinyl)-2-methyl- 3-azetidinyl)-1- piperazinyl)-2-propen-1- one | E-1 | 606.3 |
| 2-361 | 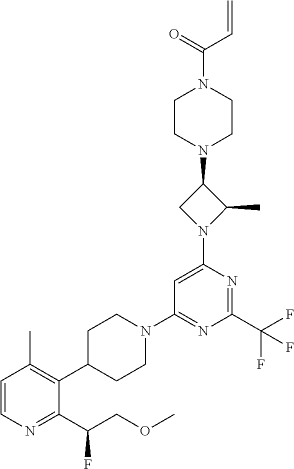 [SMILES: C=CC(=O)N1CCN([C@@H]2CN(c3cc(N4CCC(c5c(C)ccnc5[C@H](F)COC)CC4)nc(C(F)(F)F)n3)[C@@H]2C)CC1] | 1-(4-((2R,3R)-1-(6-(4- (2-((1S)-1-fluoro-2- methoxyethyl)-4- methyl-3-pyridinyl)-1- piperidinyl)-2- (trifluoromethyl)-4- pyrimidinyl)-2-methyl- 3-azetidinyl)-1- piperazinyl)-2-propen-1- one | E-1 | 606.3 |
| 2-263 | 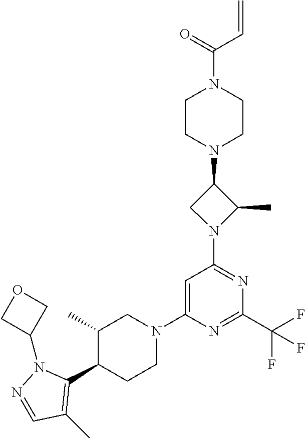 [SMILES: C=CC(=O)N1CCN([C@@H]2CN(c3cc(N4CC[C@@H](c5c(C)cnn5C5COC5)[C@H](C)C4)nc(C(F)(F)F)n3)[C@@H]2C)CC1] | 1-(4-((2R,3R)-2-methyl- 1-(6-((3S,4R)-3-methyl- 4-(4-methyl-1-(3- oxetanyl)-1H-pyrazol-5- yl)-1-piperidinyl)-2- (trifluoromethyl)-4- pyrimidinyl)-3- azetidinyl)-1- piperazinyl)-2-propen-1- one | E-8 | 535.0 |
| 2-364 | 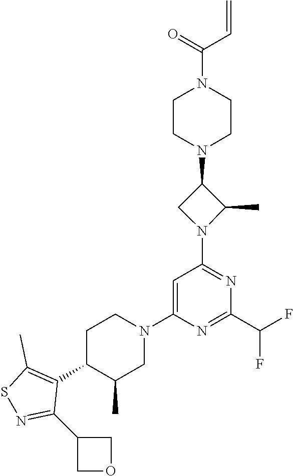 [SMILES: C=CC(=O)N1CCN([C@@H]2CN(c3cc(N4CC[C@@H](c5c(C6COC6)nsc5C)[C@H](C)C4)nc(C(F)F)n3)[C@@H]2C)CC1] | 1-(4-((2R,3R)-1-(2- (difluoromethyl)-6- ((3S,4R)-3-methyl-4-(5- methyl-3-(3-oxetanyl)- 1,2-thiazol-4-yl)-1- piperidinyl)-4- pyrimidinyl)-2-methyl- 3-azetidinyl)-1- piperazinyl)-2-propen-1- one | E-2 | 588.0 |
| 2-365 | 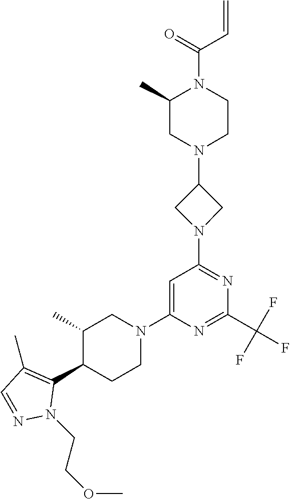 [SMILES: C=CC(=O)N1CCN(C2CN(c3cc(N4CC[C@@H](c5c(C)cnn5CCOC)[C@H](C)C4)nc(C(F)(F)F)n3)C2)C[C@H]1C] | 1-((2R)-4-(1-(6- ((3S,4R)-4-(1-(2- methoxyethyl)-4- methyl-1H-pyrazol-5- yl)-3-methyl-1- piperidinyl)-2- (trifluoromethyl)-4- pyrimidinyl)-3- azetidinyl)-2-methyl-1- piperazinyl)-2-propen-1- one | E-4 | 591.1 |
| 2-366 | 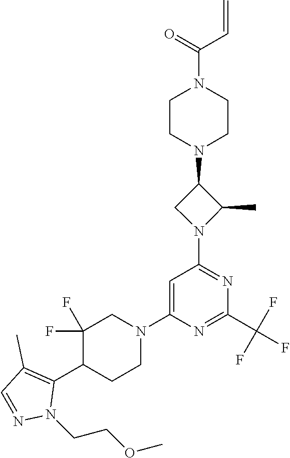 [SMILES: C=CC(=O)N1CCN([C@@H]2CN(c3cc(N4CCC(c5c(C)cnn5CCOC)C(F)(F)C4)nc(C(F)(F)F)n3)[C@@H]2C)CC1] | 1-(4-((2R,3R)-1-(6-(3,3- difluoro-4-(1-(2- methoxyethyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 613.3 |
| 2-368 | 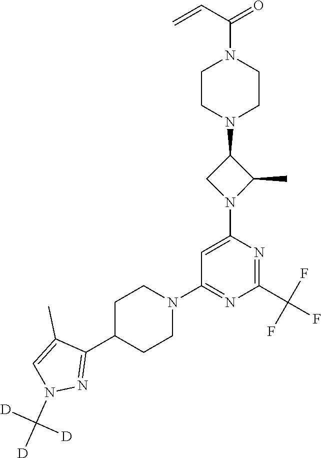 [SMILES: [2H]C([2H])([2H])n1cc(C)c(C2CCN(c3cc(N4C[C@@H](N5CCN(C(=O)C=C)CC5)[C@H]4C)nc(C(F)(F)F)n3)CC2)n1] | 1-(4-((2R,3R)-2-methyl- 1-(6-(4-(4-methyl-1- (~2~H_3_)methyl-1H- pyrazol-3-yl)-1- piperidinyl)-2- (trifluoromethyl)-4- pyrimidinyl)-3- azetidinyl)-1- piperazinyl)-2-propen-1- one | E-1 | 536.5 |
| 2-369 | 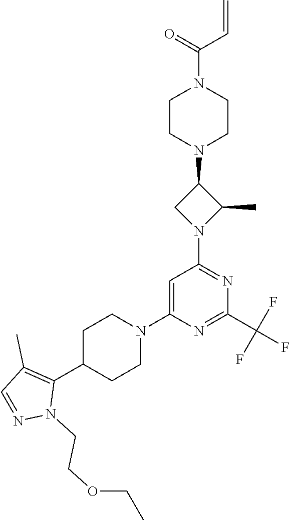 [SMILES: C=CC(=O)N1CCN([C@@H]2CN(c3cc(N4CCC(c5c(C)cnn5CCOCC)CC4)nc(C(F)(F)F)n3)[C@@H]2C)CC1] | 1-(4-((2R,3R)-1-(6-(4- (1-(2-ethoxyethyl)-4- methyl-1H-pyrazol-5- yl)-1-piperidinyl)-2- (trifluoromethyl)-4- pyrimidinyl)-2-methyl- 3-azetidinyl)-1- piperazinyl)-2-propen-1- one | E-1 | 591.0 |
| 2-370 | 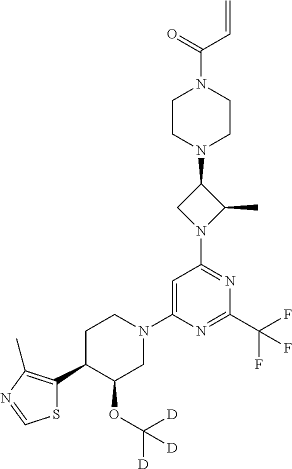 [SMILES: [2H]C([2H])([2H])O[C@@H]1CN(c2cc(N3C[C@@H](N4CCN(C(=O)C=C)CC4)[C@H]3C)nc(C(F)(F)F)n2)CC[C@@H]1c1scnc1C] | 1-(4-((2R,3R)-2-methyl- 1-(6-((3R,4R)-3- (methyloxy-d₃)-4-(4- methyl-1,3-thiazol-5- yl)-1-piperidinyl)-2- (trifluoromethyl)-4- pyrimidinyl)-3- azetidinyl)-1- piperazinyl)-2-propen-1- one | E-1 | 569.0 |
| | mixture of cis isomers at piperidine | | | |

### Section 3: Biological Evaluation

Provided in this section is the biological evaluation of the specific examples provided herein. See Table 3.

### Coupled Nucleotide Exchange Assay

The KRAS^{G12C} coupled nucleotide exchange assay allows for the screening and profiling of KRAS^{G12C} antagonists/inhibitors by monitoring the binding of an effector protein (e.g., a Ras binding domain of Raf1, RBD-cRaf) to KRAS^{G12C}. Purified GDP-bound KRAS protein (aa 1-169). containing both G12C and C118A amino acid substitutions and an N-terminal His-tag, was pre-incubated in assay buffer (25 mM HEPES pH 7.4, 10 mM MgCl₂, and 0.01% Triton X-100) with serially diluted compound for 2 h. For all subsequent steps, DTT was added to the reaction buffer at a final concentration of 1 mM. Following compound pre-incubation, purified SOS protein (aa 564-1049) and GTP (Roche 10106399001) were added to the assay wells and incubated for an additional 30 min. To determine the extent of inhibition of SOS-mediated nucleotide exchange, purified GST-tagged cRAF (aa 1-149), nickel chelate AlphaLISA acceptor beads (PerkinElmer AL108R), and AlphaScreen glutathione donor beads (PerkinElmer 6765302) were added to the assay wells and incubated for 5 min. The assay plates were then read on a plate reader measuring luminescence signal. Signal intensity of compound-containing wells were normalized to DMSO control, and data were analyzed using a 4-parameter logistic model to calculate IC₅₀ values.

### Cell Viability Assay

MIA PaCa-2 (human pancreatic carcinoma; ATCC CRL-1420) or A549 (human lung carcinoma; ATCC CCL-185) cells were cultured in RPMI 1640 medium containing 10% fetal bovine serum and 1× penicillin/streptomycin/L-glutamine. Cells were seeded in 384-well plates at a density of 1.67 E+04 cells/mL and incubated at 37° C., 5% CO₂, overnight. Serially-diluted compound or DMSO was added to the cells, and plates were incubated at 37° C., 5% CO₂ for 72 h. Cell viability was measured using a CellTiter-Glo®: Luminescent Cell Viability Assay kit (Promega) according to the manufacturer's protocol. The luminescence signal of treated samples was normalized to DMSO control, and data were analyzed using a 4-parameter logistic model to calculate IC₅₀ values.

TABLE 3
BIOCHEMICAL AND CELLULAR ACTIVITY OF EXAMPLES

| | Coupled Exchange | MIA PaCa-2, IC₅₀ | A549, IC₅₀ |
|---|---|---|---|
| Example # | IC₅₀ (μM) | (μM) | (μM) |
| 2-001 | 0.974 | 0.376 | >5 |
| 2-002 | 0.049 | 0.111 | >5 |
| 2-003 | 0.166 | 0.247 | >5 |
| 2-004 | 0.407 | 0.838 | >5 |
| 2-005 | 0.274 | 0.19 | >5 |
| 2-006 | 0.062 | 0.248 | >5 |
| 2-007 | 0.211 | 0.604 | >5 |
| 2-008 | 0.712 | 0.642 | >5 |
| 2-009 | 0.162 | 1.15 | >5 |
| 2-009-1 | 0.297 | 0.295 | >5 |
| 2-009-2 | 0.618 | NT | NT |
| 2-010 | 0.029 | 0.145 | >5 |
| 2-011 | 0.088 | 0.135 | >5 |
| 2-012 | 0.067 | 0.174 | 3.44 |
| 2-013 | 0.067 | 0.131 | >5 |
| 2-014 | 0.075 | 0.264 | >5 |
| 2-015 | 0.152 | 0.437 | >5 |
| 2-017 | 0.187 | 0.346 | >5 |
| 2-018 | 0.192 | 0.795 | >5 |
| 2-019 | 0.207 | 0.477 | >5 |
| 2-020 | 0.224 | NT | NT |
| 2-021 | 0.387 | 0.712 | >5 |
| 2-022 | 0.428 | 0.381 | >5 |
| 2-023 | 0.478 | 1.57 | >5 |
| 2-024 | 0.588 | 0.932 | >5 |
| 2-025-1 | 0.307 | 1.760 | >5 |
| 2-025-2 | 0.416 | 3.210 | >5 |
| 2-026 | 0.052 | 0.091 | >5 |
| 2-027 | 0.052 | 0.112 | >5 |
| 2-028 | 0.053 | 0.25 | >5 |
| 2-029 | 0.057 | 0.327 | >5 |
| 2-030 | 0.109 | 0.295 | >5 |
| 2-030-1 | 0.084 | 0.277 | >5 |
| 2-030-2 | 0.591 | 1.37 | >5 |
| 2-031 | 0.085 | 0.274 | >5 |
| 2-032-1 | 0.089 | 0.139 | >5 |
| 2-032-2 | 2.8 | NT | NT |
| 2-033 | 0.114 | 0.483 | >5 |
| 2-034 | 0.119 | 1.06 | >5 |
| 2-035 | 0.122 | 0.368 | >5 |
| 2-036 | 0.155 | NT | NT |
| 2-036-1 | 0.136 | NT | NT |
| 2-036-2 | 0.646 | 1.56 | >5 |
| 2-037 | 0.171 | 0.308 | >5 |
| 2-038-1 | 1.24 | NT | NT |
| 2-038-2 | 0.262 | 0.315 | >5 |
| 2-039 | 0.304 | 1.27 | >5 |
| 2-040 | 0.313 | 0.85 | >5 |
| 2-041 | 0.427 | 1.06 | >5 |
| 2-042-1 | 22.7 | NT | NT |
| 2-042-2 | 0.456 | 0.57 | >5 |
| 2-043 | 0.714 | 0.748 | >5 |
| 2-044 | 0.14 | 0.843 | >5 |
| 2-045 | 0.144 | 0.131 | >5 |
| 2-046 | 0.162 | 0.276 | >5 |
| 2-047 | 0.177 | 0.709 | >5 |
| 2-048 | 0.246 | 0.237 | >5 |
| 2-049 | 0.158 | 0.284 | >5 |
| 2-050 | 0.241 | 0.716 | >5 |
| 2-051 | 0.555 | 1.11 | >5 |
| 2-052 | 0.153 | 0.531 | >5 |
| 2-053 | 0.351 | 0.613 | >5 |
| 2-054 | 0.1 | 0.277 | >5 |
| 2-055 | 0.258 | 1.35 | >5 |
| 2-056 | 0.293 | 0.988 | >5 |
| 2-057 | 0.271 | 0.222 | >5 |
| 2-058 | 0.369 | NT | NT |
| 2-059 | 0.415 | 0.382 | >5 |
| 2-060 | 0.177 | 0.186 | >5 |
| 2-061 | 0.384 | 0.951 | >5 |
| 2-062 | 0.164 | 0.207 | >5 |
| 2-063 | 0.723 | 0.492 | 14.8 |
| 2-065 | 0.457 | 1.54 | 10.4 |
| 2-066 | 0.397 | 1.52 | >5 |
| 2-067 | 0.221 | 0.261 | >5 |
| 2-068 | 0.281 | 0.342 | >5 |
| 2-069 | 0.283 | 1.08 | >5 |
| 2-070 | 0.078 | 0.398 | >5 |
| 2-071 | 0.355 | 0.66 | >5 |
| 2-073 | 0.152 | 0.354 | >5 |
| 2-074 | 0.145 | 0.876 | >5 |
| 2-075 | 0.096 | 0.504 | >5 |
| 2-076 | 0.067 | 0.168 | >5 |
| 2-077 | 0.343 | 0.518 | >5 |
| 2-078 | 0.222 | 0.448 | >5 |
| 2-079 | 0.302 | 25 | >25 |
| 2-080 | 0.393 | 5 | >5 |
| 2-081 | 0.057 | 5 | >5 |
| 2-082 | 18.9 | NT | NT |
| 2-083 | 0.748 | 0.496 | >5 |
| 2-084 | 0.895 | 2.71 | >5 |
| 2-084-1 | 1.0 | NT | NT |
| 2-084-2 | 14.4 | NT | NT |
| 2-085 | 0.723 | 0.761 | >5 |
| 2-086 | 0.484 | 0.757 | >5 |
| 2-087 | 0.084 | 0.194 | >5 |
| 2-088 | 0.209 | 0.235 | 25 |
| 2-089 | 0.381 | 0.535 | >5 |
| 2-090 | 0.064 | 0.231 | >5 |
| 2-091 | 0.38 | 0.725 | >5 |
| 2-092 | 0.426 | 1.28 | >5 |
| 2-093 | 0.24 | 1.22 | >5 |
| 2-094 | 0.06 | 0.165 | >5 |
| 2-095 | 0.039 | 0.113 | >5 |
| 2-096 | 0.135 | 0.204 | >25 |
| 2-097 | 0.124 | 0.125 | >25 |
| 2-098 | 0.675 | 0.678 | 17.5 |
| 2-099 | 0.197 | 0.512 | >25 |
| 2-100 | 0.431 | 0.923 | >5 |
| 2-101 | 1.229 | NT | NT |
| 2-101-1 | 0.355 | NT | NT |
| 2-101-2 | 9.92 | NT | NT |
| 2-102 | 0.137 | 0.31 | >5 |
| 2-103 | 0.099 | 0.667 | >5 |
| 2-104 | 0.083 | 0.083 | >5 |
| 2-105 | 0.436 | 0.327 | >5 |
| 2-106 | 0.089 | 0.161 | >5 |
| 2-107 | 0.069 | 0.078 | >5 |
| 2-108 | 0.039 | 0.167 | >5 |
| 2-109 | 0.058 | 0.236 | >5 |
| 2-110 | 0.096 | 0.265 | >5 |
| 2-111 | 0.212 | 0.424 | >5 |
| 2-112 | 0.149 | 0.208 | >5 |
| 2-113 | 0.219 | 0.15 | >5 |
| 2-114 | 0.051 | 0.131 | >5 |
| 2-115 | 0.03 | 0.1 | >5 |
| 2-116 | 0.175 | 0.344 | >5 |
| 2-117 | 0.108 | 0.275 | >5 |
| 2-118 | 0.256 | 0.504 | >5 |
| 2-119 | 0.295 | 0.708 | >5 |
| 2-120 | 0.062 | 0.2 | >5 |
| 2-121 | 0.061 | 0.258 | >5 |
| 2-122 | 0.106 | 0.142 | >5 |
| 2-123 | 0.337 | 0.376 | >5 |
| 2-124 | 0.344 | 0.23 | >5 |
| 2-125 | 0.306 | 0.296 | >5 |
| 2-126 | 0.29 | 0.98 | >5 |
| 2-127 | 0.227 | 0.115 | >25 |
| 2-128 | 0.19 | 0.104 | >25 |
| 2-129 | 0.13 | 0.145 | >25 |
| 2-130 | 0.186 | 3.15 | 8 |
| 2-131 | 0.225 | 5.1 | 8.9 |
| 2-132 | 0.195 | 2.85 | >25 |
| 2-133 | 0.203 | 9.75 | 15.1 |
| 2-134 | 0.973 | 1.96 | 11.9 |
| 2-135 | 0.218 | 0.291 | 14.7 |
| 2-136 | 0.01 | 0.057 | >5 |
| 2-137 | 0.279 | 0.263 | 17.6 |
| 2-138 | 0.96 | 1.51 | >5 |
| 2-139-1 | 1.98 | NT | NT |
| 2-139-2 | 0.923 | NT | NT |
| 2-140 | 0.675 | NT | NT |
| 2-140-1 | 17.1 | NT | NT |
| 2-140-2 | 0.695 | NT | NT |
| 2-141 | 0.078 | 0.237 | 4.22 |
| 2-142 | 0.081 | 0.397 | >5 |
| 2-143 | 0.089 | 0.316 | >5 |
| 2-144 | 0.106 | 0.185 | 10.4 |
| 2-145 | 0.118 | 0.187 | >5 |
| 2-146 | 0.171 | 0.202 | >5 |
| 2-147 | 0.226 | 0.829 | 5.51 |
| 2-148 | 0.251 | 0.408 | >5 |
| 2-150 | 0.288 | NT | NT |
| 2-151 | 0.317 | 12.6 | 25 |
| 2-152 | 0.322 | 0.433 | >5 |
| 2-153 | 0.411 | 16.4 | >25 |
| 2-154 | 0.451 | 1.28 | 7.1 |
| 2-155 | 0.466 | 0.556 | >5 |
| 2-156 | 0.472 | 1.2 | >5 |
| 2-157 | 0.508 | 1.77 | >5 |
| 2-158 | 0.509 | 1.1 | >5 |
| 2-159 | 0.554 | 2.01 | >5 |
| 2-160 | 0.594 | 0.849 | >5 |
| 2-161 | 0.603 | 1.11 | >5 |
| 2-162 | 0.606 | 0.448 | >5 |
| 2-163 | 0.655 | 0.547 | 11.6 |
| 2-164 | 0.669 | 1 | >5 |
| 2-165 | 0.675 | 2.01 | >5 |
| 2-167 | 0.826 | 0.96 | 13.2 |
| 2-168 | 0.86 | 0.568 | 12.4 |
| 2-169 | 0.888 | NT | NT |
| 2-170 | 0.893 | 3.44 | >5 |
| 2-171 | 0.929 | 0.457 | >25 |
| 2-172 | 0.935 | NT | NT |
| 2-173 | 0.938 | 0.53 | 16.1 |
| 2-174 | 0.986 | NT | NT |
| 2-175 | 0.691 | NT | NT |
| 2-176 | 0.221 | 0.196 | >5 |
| 2-177 | 0.659 | NT | NT |
| 2-178 | 0.099 | >5 | >5 |
| 2-179 | 0.101 | 0.099 | >5 |
| 2-180 | 0.048 | 0.075 | >5 |
| 2-181 | 0.096 | 0.127 | >5 |
| 2-182 | 0.026 | 0.085 | >5 |
| 2-184 | 0.141 | 0.148 | >5 |
| 2-185 | 0.738 | NT | NT |
| 2-186 | 0.185 | >5 | >5 |
| 2-187 | 0.047 | 0.063 | >5 |
| 2-188 | 0.100 | 0.099 | >5 |
| 2-189 | 0.189 | 0.309 | >5 |
| 2-190 | 0.109 | 0.263 | >5 |
| 2-191 | 0.887 | NT | NT |
| 2-192 | 0.826 | NT | NT |
| 2-193 | 0.970 | NT | NT |
| 2-194 | 0.108 | >5 | >5 |
| 2-195 | 0.154 | 0.202 | >5 |
| 2-196 | 0.053 | 0.055 | >5 |
| 2-197 | 0.199 | 0.277 | >5 |
| 2-198 | 0.045 | 0.12 | >5 |
| 2-199 | 0.051 | 0.158 | >5 |
| 2-200 | 0.633 | 2.27 | >5 |
| 2-201 | 0.123 | 0.208 | >5 |
| 2-202 | 0.059 | 0.332 | >5 |
| 2-203 | 0.798 | NT | NT |
| 2-204 | 0.477 | 0.524 | >5 |
| 2-205 | 0.241 | 0.291 | >2.5 |
| 2-206 | 0.221 | 0.32 | >5 |
| 2-207 | 0.104 | 0.186 | >5 |
| 2-208 | 0.265 | 0.398 | >5 |
| 2-209 | 0.086 | 0.221 | 2.81 |
| 2-210 | 0.790 | NT | NT |
| 2-211 | 0.197 | 0.443 | >5 |
| 2-212 | 0.047 | 0.14 | >5 |
| 2-213 | 0.102 | 0.204 | >5 |
| 2-214 | 0.177 | 0.32 | >5 |
| 2-215 | 0.098 | 0.211 | >5 |
| 2-216 | 0.075 | 0.195 | >5 |
| 2-217 | 0.201 | 0.397 | >5 |
| 2-218 | 0.099 | 0.249 | >5 |
| 2-219 | 0.164 | 0.337 | >5 |
| 2-220 | 0.193 | 0.279 | >5 |
| 2-221 | 0.071 | 0.157 | >5 |
| 2-222 | 0.070 | 0.149 | >5 |
| 2-223 | 0.065 | 0.164 | >5 |
| 2-224 | 0.094 | 0.17 | >5 |
| 2-225 | 0.498 | 0.647 | >5 |
| 2-226 | 0.112 | 0.211 | >5 |
| 2-227 | 0.179 | 0.238 | >5 |
| 2-228 | 0.753 | NT | NT |
| 2-229 | 0.068 | 0.435 | >5 |
| 2-230 | 0.392 | 0.372 | >5 |
| 2-231 | 0.755 | NT | NT |
| 2-232 | 0.556 | NT | NT |
| 2-233 | 0.163 | 0.202 | >5 |
| 2-234 | 0.420 | 0.676 | >5 |
| 2-235 | 0.935 | NT | NT |
| 2-236 | 0.117 | 0.236 | >5 |
| 2-237 | 0.265 | 0.438 | >5 |
| 2-238 | 0.072 | 0.126 | >5 |
| 2-239 | 0.159 | 0.282 | >5 |
| 2-240 | 0.400 | 0.576 | >5 |
| 2-241 | 0.191 | 0.373 | >5 |
| 2-242 | 0.278 | 0.429 | >5 |
| 2-243 | 0.093 | 0.067 | >5 |
| 2-244 | 0.440 | 1.14 | >5 |
| 2-245 | 0.106 | 0.196 | >5 |
| 2-246 | 0.125 | 0.16 | >5 |
| 2-247 | 0.223 | 0.184 | >5 |
| 2-248 | 0.384 | 0.421 | >5 |
| 2-249 | 0.129 | 0.152 | >5 |
| 2-250 | 0.217 | 0.347 | >5 |
| 2-251 | 0.165 | 0.268 | >5 |
| 2-252 | 0.242 | 0.245 | >5 |
| 2-253 | 0.296 | 0.366 | >5 |
| 2-254 | 0.264 | 0.513 | >5 |
| 2-255 | 0.075 | 0.158 | >5 |
| 2-256 | 0.346 | 0.577 | >5 |
| 2-257 | 0.464 | 0.654 | >5 |
| 2-258 | 0.177 | 0.269 | >5 |
| 2-259 | 0.068 | 0.126 | >5 |
| 2-260 | 0.282 | 0.254 | >5 |
| 2-261 | 0.119 | 0.13 | >5 |
| 2-262 | 0.318 | 0.313 | >5 |
| 2-263 | 0.023 | 0.045 | >5 |
| 2-264 | 0.431 | 0.324 | >5 |
| 2-265 | 0.208 | 0.167 | >5 |
| 2-266 | 0.096 | 0.045 | >5 |
| 2-267 | 0.134 | 0.359 | >5 |
| 2-268 | 0.105 | 0.184 | >5 |
| 2-269 | 0.097 | 0.195 | >5 |
| 2-270 | 0.221 | 0.227 | >5 |
| 2-271 | 0.268 | 0.268 | >5 |
| 2-272 | 0.801 | NT | NT |
| 2-273 | 0.362 | 0.608 | >5 |
| 2-274 | 0.494 | 0.539 | >5 |
| 2-275 | 0.402 | 0.875 | >5 |
| 2-276 | 0.270 | 0.285 | >5 |
| 2-277 | 0.168 | 0.233 | >5 |
| 2-278 | 0.357 | 0.638 | >5 |
| 2-279 | 0.234 | 0.293 | >5 |
| 2-280 | 0.148 | 0.151 | >5 |
| 2-281 | 0.131 | 0.171 | >5 |
| 2-282 | 0.635 | NT | NT |
| 2-283 | 0.250 | 0.182 | >5 |
| 2-284 | 0.310 | 0.485 | >5 |
| 2-285 | 0.473 | 0.637 | >5 |
| 2-286 | 0.827 | NT | NT |
| 2-287 | 0.036 | 0.079 | >5 |
| 2-288 | 0.706 | NT | NT |
| 2-289 | 0.139 | 0.179 | >5 |
| 2-290 | 0.124 | 0.178 | >5 |
| 2-291 | 0.192 | 0.296 | >5 |
| 2-292 | 0.486 | NT | NT |
| 2-293 | 0.421 | 0.48 | >5 |
| 2-294 | 0.231 | 0.229 | >5 |
| 2-295 | 0.370 | 0.564 | >5 |
| 2-296 | 0.199 | 0.335 | >5 |
| 2-297 | 0.467 | 0.72 | >5 |
| 2-298 | 0.141 | 0.254 | >5 |
| 2-299 | 0.083 | 0.106 | >5 |
| 2-300 | 0.143 | 1.1 | >5 |
| 2-301 | 0.181 | 0.225 | >5 |
| 2-302 | 0.165 | 0.239 | >5 |
| 2-303 | 0.803 | NT | NT |
| 2-304 | 0.823 | NT | NT |
| 2-305 | 0.221 | 0.305 | >5 |
| 2-306 | 0.324 | 0.573 | >5 |
| 2-307 | 0.109 | 0.5 | 3.05 |
| 2-308 | 0.041 | 0.256 | 4.48 |
| 2-309 | 0.223 | 1.1 | 2.71 |
| 2-310 | 0.248 | 0.337 | >5 |
| 2-311 | 0.481 | 1.41 | >5 |
| 2-312 | 0.270 | 0.298 | >5 |
| 2-313 | 0.185 | 0.253 | >5 |
| 2-314 | 0.169 | 0.197 | >5 |
| 2-315 | 0.532 | 0.575 | >5 |
| 2-316 | 0.697 | NT | NT |
| 2-317 | 0.092 | 0.164 | >5 |
| 2-318 | 0.090 | 0.141 | >5 |
| 2-319 | 0.198 | 0.255 | >5 |
| 2-320 | 0.047 | 0.119 | >5 |
| 2-321 | 0.142 | 0.214 | >5 |
| 2-322 | 0.054 | 0.094 | >5 |
| 2-323 | 0.189 | NT | NT |
| 2-324 | 0.920 | NT | NT |
| 2-325 | 0.139 | 0.207 | >5 |
| 2-326 | 0.547 | 0.956 | >5 |
| 2-327 | 0.232 | 0.44 | >5 |
| 2-328 | 0.455 | 0.651 | >5 |
| 2-329 | 0.048 | 0.056 | >5 |
| 2-330 | 0.140 | 0.24 | >5 |
| 2-331 | 0.589 | NT | NT |
| 2-332 | 0.483 | 0.556 | >5 |
| 2-333 | 0.158 | 0.315 | >5 |
| 2-334 | 0.129 | 0.244 | >5 |
| 2-335 | 0.877 | NT | NT |
| 2-336 | 0.142 | 0.232 | >5 |
| 2-337 | 0.496 | NT | NT |
| 2-338 | 0.957 | NT | NT |
| 2-339 | 0.643 | NT | NT |
| 2-340 | 0.049 | 0.074 | >5 |
| 2-341 | 0.172 | 0.593 | >5 |
| 2-342 | 0.221 | 0.306 | >5 |
| 2-343 | 0.288 | NT | NT |
| 2-344 | 0.766 | NT | NT |
| 2-345 | 0.478 | 0.504 | >5 |
| 2-346 | 0.249 | 0.53 | >5 |
| 2-347 | 0.070 | 0.174 | >5 |
| 2-348 | 0.288 | 0.389 | >5 |
| 2-349 | 0.101 | 0.144 | >5 |
| 2-350 | 0.203 | NT | NT |
| 2-351 | 0.541 | NT | NT |
| 2-352 | 0.053 | NT | NT |
| 2-353 | 0.061 | NT | NT |
| 2-354 | 0.695 | NT | NT |
| 2-355 | 0.433 | 0.579 | >5 |
| 2-356 | 0.476 | 0.915 | >5 |
| 2-357 | 0.747 | NT | NT |
| 2-358 | 0.466 | NT | NT |
| 2-359 | 0.586 | NT | NT |
| 2-360 | 0.085 | NT | NT |
| 2-361 | 0.140 | NT | NT |
| 2-362 | 0.083 | NT | NT |
| 2-363 | 0.162 | NT | NT |
| 2-364 | 0.113 | NT | NT |
| 2-365 | 0.252 | NT | NT |
| 2-366 | 0.203 | NT | NT |
| 2-367 | 0.547 | NT | NT |
| 2-368 | 0.204 | NT | NT |
| 2-369 | 0.152 | NT | NT |
| 2-370 | 0.713 | NT | NT |
| 2-371 | 0.042 | 0.074 | >5.0 |
| 2-372 | 0.009 | 0.031 | >5.0 |
| 2-373 | 0.011 | 0.050 | >5.0 |
| 2-374 | 0.024 | 0.088 | >5.0 |
| 2-375 | 0.026 | 0.071 | >5.0 |
| 2-376 | 0.016 | 0.045 | >5.0 |
| 2-377 | 0.017 | 0.039 | >5.0 |
| 2-378 | 0.042 | 0.066 | >5.0 |
| 2-378-1 | 0.029 | 0.058 | >5.0 |
| 2-378-2 | 0.475 | NT | NT |
| 2-379 | 0.029 | 0.107 | >5.0 |
| 2-380 | 0.033 | 0.151 | >5.0 |
| 2-382 | 0.046 | 0.128 | >5.0 |
| 2-383 | 0.053 | 0.14 | >5.0 |
| 2-384 | 0.078 | 0.179 | >5.0 |
| 2-385 | 0.084 | 0.378 | >5.0 |
| 2-386 | 0.092 | 0.279 | >5.0 |
| 2-387 | 0.127 | 0.33 | >5.0 |
| 2-388 | 0.141 | 0.338 | >5.0 |
| 2-389 | 0.191 | 0.376 | >5.0 |
| 2-390 | 0.416 | 0.695 | >5.0 |
| 2-391 | 0.084 | 0.278 | >5.0 |
| 2-392 | 0.079 | 0.205 | >5.0 |
| 2-392-1 | 0.042 | 0.142 | >5.0 |
| 2-393 | 0.024 | 0.099 | >5.0 |
| 2-394-1 | 0.215 | NT | NT |
| 2-394-2 | 0.072 | 0.261 | >5.0 |
| 2-394-3 | 0.024 | 0.067 | >5.0 |
| 2-394-4 | 0.0674 | 0.236 | >5.0 |
| 2-395-1 | 0.027 | 0.091 | >5.0 |
| 2-395-2 | 0.908 | NT | NT |
| 2-396 | 0.041 | 0.126 | >5.0 |
| 2-397-1 | 0.114 | 0.306 | >5.0 |
| 2-397-2 | 0.141 | 0.612 | >5.0 |
| 2-397-3 | 0.045 | 0.118 | >5.0 |
| 2-397-4 | 0.245 | NT | NT |
| 2-398 | 0.045 | 0.149 | >5.0 |
| 2-399 | 0.045 | 0.109 | >5.0 |
| 2-400-1 | 0.206 | 0.601 | >5.0 |
| 2-400-2 | 0.046 | 0.126 | >5.0 |
| 2-401 | 0.049 | 0.085 | >5.0 |
| 2-402 | 0.051 | 0.133 | >5.0 |
| 2-403 | 0.056 | 0.090 | >5.0 |
| 2-404 | 0.105 | 0.242 | >5.0 |
| 2-405 | 0.114 | 0.242 | >5.0 |
| 2-406 | 0.135 | 0.176 | >5.0 |
| 2-407 | 0.139 | 0.294 | >5.0 |
| 2-408 | 0.189 | 0.402 | >5.0 |
| 2-409-1 | 0.57 | NT | NT |
| 2-409-2 | 0.226 | 0.429 | >5.0 |
| 2-410 | 0.236 | NT | NT |
| 2-411 | 0.762 | NT | NT |
| 2-412 | 0.318 | 0.554 | >5.0 |
| 2-413 | 0.316 | 0.091 | >5.0 |
| 2-414 | 0.317 | 0.713 | >5.0 |
| 2-415 | 0.573 | NT | NT |
| 2-416 | 0.084 | 0.266 | >5.0 |
| 2-417 | 0.436 | NT | NT |
| 2-418 | 0.919 | NT | NT |
| 2-419 | 0.491 | NT | NT |
| 2-420 | 0.043 | 0.121 | >5.0 |
| 2-421 | 0.068 | 0.316 | >5.0 |
| 2-422 | 0.13 | 0.318 | >5.0 |
| 2-423 | 0.268 | 0.335 | >5.0 |
| 2-424 | 0.739 | NT | NT |
| 2-425-1 | 0.060 | 0.214 | >5.0 |
| 2-426 | 0.116 | 0.169 | >5.0 |
| 2-427 | 0.463 | NT | NT |
| 2-428-1 | 0.237 | 0.626 | >5.0 |
| 2-428-2 | 0.056 | 0.226 | >5.0 |
| 2-429 | 0.157 | NT | NT |
| 2-430 | 0.355 | 0.638 | >5.0 |
| 2-431 | 0.011 | 0.049 | >5.0 |
| 2-432 | 0.016 | 0.071 | >5.0 |
| 2-433 | 0.028 | 0.089 | >5.0 |
| 2-434 | 0.033 | 0.076 | >5.0 |
| 2-435 | 0.039 | 0.096 | >5.0 |
| 2-436 | 0.039 | 0.066 | >5.0 |
| 2-437 | 0.048 | 0.137 | >5.0 |
| 2-438 | 0.052 | 0.126 | >5.0 |
| 2-439 | 0.059 | 0.089 | >5.0 |
| 2-440 | 0.061 | 0.162 | >5.0 |
| 2-441 | 0.066 | 0.269 | >5.0 |
| 2-442 | 0.069 | 0.152 | >5.0 |
| 2-443 | 0.075 | 0.128 | >5.0 |
| 2-444 | 0.075 | 0.275 | >5.0 |
| 2-445 | 0.083 | 0.318 | >5.0 |
| 2-446 | 0.091 | 0.196 | >5.0 |
| 2-447 | 0.127 | 0.168 | >5.0 |
| 2-448 | 0.130 | 0.38 | >5.0 |
| 2-449 | 0.136 | 0.221 | >5.0 |
| 2-450 | 0.139 | NT | NT |
| 2-451 | 0.149 | 0.253 | >5.0 |
| 2-452 | 0.181 | 0.904 | >5.0 |
| 2-453 | 0.193 | 0.207 | >5.0 |
| 2-454 | 0.221 | NT | NT |
| 2-455 | 0.222 | NT | NT |
| 2-456 | 0.35 | 0.446 | >5.0 |
| 2-457-1 | 0.355 | NT | NT |
| 2-458 | 0.374 | 1.01 | >5.0 |
| 2-459 | 0.385 | NT | NT |
| 2-460 | 0.539 | NT | NT |
| 2-461 | 0.555 | NT | NT |
| 2-462 | 0.753 | NT | NT |
| 2-463 | 0.815 | NT | NT |
| 2-464 | 0.828 | NT | NT |
| 2-465 | 0.951 | NT | NT |
| 2-466-1 | 0.670 | NT | NT |
| 2-466-2 | 0.024 | 0.131 | >5.0 |
| 2-467 | 0.606 | NT | NT |
| 2-468 | 0.044 | 0.112 | >5.0 |
| 2-469 | 0.042 | 0.13 | >5.0 |
| 2-470 | 0.099 | 0.114 | >5.0 |
| 2-471 | 0.099 | 0.128 | >5.0 |
| 2-472 | 0.067 | 0.235 | >5.0 |
| 2-473 | 0.161 | 0.647 | >5.0 |
| 2-474 | 0.505 | NT | NT |
| 2-475 | 0.69 | NT | NT |
| 2-476 | 0.761 | NT | NT |
| 2-477 | 0.070 | 0.163 | >5.0 |
| 2-477-1 | 0.029 | 0.083 | >5.0 |
| 2-478 | 0.141 | 0.156 | >5.0 |
| 2-478-1 | 0.050 | 0.080 | >5.0 |
| 2-479 | 0.107 | 0.118 | >5.0 |
| 2-479-1 | 0.054 | 0.112 | >5.0 |
| 2-480 | 0.422 | NT | NT |
| 2-481 | 0.47 | NT | NT |
| 2-482 | 0.807 | NT | NT |
| 2-483 | 0.863 | NT | NT |
| 2-484 | 0.124 | NT | NT |
| 2-485 | 0.614 | NT | NT |
| 2-486 | 0.092 | 0.174 | >5.0 |
| 2-487 | 0.099 | 0.154 | >5.0 |
| 2-488 | 0.106 | 0.193 | >5.0 |
| 2~489 | 0.117 | 0.366 | >5.0 |
| 2-489-1 | 0.060 | 0.179 | >5.0 |
| 2-490 | 0.089 | 0.448 | >5.0 |
| 2-490-1 | 0.080 | 0.152 | >5.0 |
| 2-491 | 0.121 | 0.233 | >5.0 |
| 2-491-1 | 0.289 | 0.556 | >5.0 |
| 2-491-2 | 0.155 | 0.24 | >5.0 |
| 2-492 | 0.187 | 0.493 | >5.0 |
| 2-492-1 | 0.089 | 0.277 | >5.0 |
| 2-493 | 0.122 | 0.21 | >5.0 |
| 2-493-1 | 0.106 | 0.169 | >5.0 |
| 2-493-2 | 0.567 | NT | NT |
| 2-493-3 | 0.32 | NT | NT |
| 2-494 | 0.26 | 0.423 | >5.0 |
| 2-494-1 | 0.066 | 0.163 | >5.0 |
| 2-495-1 | 0.182 | 0.38 | >5.0 |
| 2-496-1 | 0.547 | NT | NT |
| 2-497 | 0.399 | 0.472 | >5.0 |
| 2-498 | 0.419 | NT | NT |
| 2-499 | 0.445 | NT | NT |
| 2-500 | 0.548 | NT | NT |
| 2-501 | 0.054 | 0.189 | >5.0 |
| 2-502 | 0.012 | 0.057 | >5.0 |
| 2-503 | 0.148 | 0.454 | >5.0 |
| 2-504 | 0.042 | 0.178 | >5.0 |
| 2-505 | 0.185 | 0.32 | >5.0 |
| 2-506 | 0.039 | 0.089 | >5.0 |
| 2-507-1 | 0.079 | 0.199 | >5.0 |
| 2-507-2 | 0.070 | 0.282 | >5.0 |
| 2-508 | 0.204 | NT | NT |
| 2-509 | 0.196 | 0.457 | >5.0 |
| 2-510 | 0.113 | 0.26 | >5.0 |
| 2-510-1 | 0.020 | 0.198 | >5.0 |
| 2-510-2 | 0.041 | 0.158 | >5.0 |
| 2-511 | 0.156 | 0.291 | >5.0 |
| 2-512 | 0.177 | 0.263 | >5.0 |
| 2-513 | 0.022 | NT | NT |
| 2-514-1 | 0.032 | 0.13 | >5.0 |
| 2-515 | 0.103 | 0.338 | >5.0 |
| 2-516-1 | 0.048 | 0.133 | >5.0 |
| 2-517 | 0.078 | 0.307 | >5.0 |
| 2-518 | 0.090 | 0.216 | >5.0 |
| 2-519 | 0.143 | 0.587 | >5.0 |
| 2-520 | 0.165 | 0.383 | >5.0 |
| 2-520-1 | 0.185 | 0.892 | >5.0 |
| 2-520-2 | 0.148 | 0.488 | >5.0 |
| 2-521 | 0.157 | 0.217 | >5.0 |
| 2-522 | 0.275 | 0.814 | >5.0 |
| 2-523 | 0.285 | NT | NT |
| 2-524 | 0.323 | NT | NT |
| 2-525 | 0.329 | NT | NT |
| 2-526 | 0.709 | NT | NT |
| 2-527 | 0.066 | 0.087 | >5.0 |
| 2-528 | 0.070 | 0.081 | >5.0 |
| 2-529 | 0.304 | 0.306 | >5.0 |
| 2-530 | 0.879 | NT | NT |
| 2-531-1 | 0.096 | 0.313 | >5.0 |
| 2-532-1 | 0.272 | 0.579 | >5.0 |
| 2-533-1 | 0.454 | NT | NT |
| 2-534-1 | 0.557 | NT | NT |
| 2-535 | 0.302 | NT | NT |
| 2-536 | 0.651 | 0.659 | >5.0 |
| 2-537 | 0.805 | NT | NT |
| 2-538 | 0.568 | NT | NT |
| 2-539 | 0.041 | 0.076 | >5.0 |
| 2-540 | 0.216 | 0.233 | >5.0 |
| 2-541 | 0.037 | 0.070 | >5.0 |
| 2-542 | 0.721 | NT | NT |
| 2-543 | 0.071 | 0.136 | >5.0 |
| 2-544 | 0.066 | 0.128 | >5.0 |
| 2-545 | 0.356 | 0.873 | >5.0 |
| 2-546 | 0.127 | 0.181 | >5.0 |
| 2-547 | 0.063 | 0.075 | >>5.0 |
| 2-548 | 0.026 | 0.042 | >5.0 |
| 2-549 | 0.016 | 0.038 | >5.0 |
| 2-550 | 0.247 | 0.589 | >5.0 |
| 2-551 | 0.098 | 0.142 | >5.0 |
| 2-552 | 0.681 | NT | NT |
| 2-553 | 0.031 | 0.121 | >5.0 |
| 2-554 | 0.114 | 0.18 | >5.0 |
| 2-555 | 0.019 | 0.075 | >5.0 |
| 2-556 | 0.060 | 0.188 | >5.0 |
| 2-557 | 0.053 | 0.148 | >5.0 |
| 2-558 | 0.068 | 0.162 | >5.0 |
| 2-559 | 0.073 | 0.277 | >5.0 |
| 2-560 | 0.14 | 0.715 | >5.0 |
| 2-561 | 0.186 | 0.497 | >5.0 |
| 2-562 | 0.127 | 0.252 | >5.0 |
| 2-563 | 0.294 | 0.89 | >5.0 |
| 2-564 | 0.753 | NT | NT |
| 2-565 | 0.701 | NT | NT |
| 2-566 | 0.167 | 0.462 | >5.0 |
| 2-567 | 0.124 | 0.541 | >5.0 |
| 2-568 | 0.133 | 0.317 | >5.0 |
| 2-569 | 0.229 | 0.366 | >5.0 |
| 2-570 | 0.127 | 0.153 | >5.0 |
| 2-571 | 0.0715 | 0.852 | >5.0 |
| 2-572 | 0.11 | 1.11 | >5.0 |
| 2-573 | 0.195 | >5.0 | >5.0 |
| 2-574 | 0.144 | >5.0 | >5.0 |
| 2-575 | 0.118 | 0.345 | >5.0 |
| 2-576 | 0.060 | NT | NT |
| 2-577 | 0.089 | 0.13 | >5.0 |
| 2-578 | 0.08 | 0.137 | >5.0 |
| 2-579 | 0.266 | NT | NT |
| 2-580 | 0.126 | 0.54 | >5.0 |
| 2-581 | 0.053 | 0.183 | >5.0 |
| 2-582 | 0.059 | 0.113 | >5.0 |
| 2-583 | 0.458 | NT | NT |
| 2-584 | 0.139 | 0.458 | >5.0 |
| 2-585 | 0.043 | 0.097 | >5.0 |
| 2-586 | 0.062 | 0.189 | >5.0 |
| 2-588 | 0.046 | 0.128 | >5.0 |
| 2-589 | 0.032 | 0.087 | >5.0 |
| 2-590 | 0.189 | NT | NT |
| 2-591 | 0.076 | 0.212 | >5.0 |
| 2-592 | 0.065 | 0.362 | >5.0 |
| 2-593 | 0.081 | 0.241 | >5.0 |
| 2-594 | 0.063 | NT | NT |
| 2-595 | 0.051 | NT | NT |
| 2-596 | 0.102 | 0.182 | >5.0 |
| 2-597 | 0.028 | 0.069 | >5.0 |
| 2-598 | 0.028 | 0.063 | >5.0 |
| 2-599 | 0.1 | 0.149 | >5.0 |
| 2-600 | 0.060 | 0.176 | >5.0 |
| 2-601 | 0.097 | 0.369 | >5.0 |
| 2-602 | 0.138 | 0.487 | >5.0 |
| 2-603 | 0.065 | 0.254 | >5.0 |
| 2-604 | 0.171 | 0.432 | >5.0 |
| 2-605 | 0.051 | 0.23 | >5.0 |
| 2-606 | 0.159 | 0.369 | >5.0 |
| 2-607 | 0.121 | 0.321 | >5.0 |
| 2-608 | 0.055 | 0.178 | >5.0 |
| 2-609 | 0.091 | 0.317 | >5.0 |
| 2-610 | 0.121 | 0.38 | >5.0 |
| 2-611 | 0.111 | 0.214 | >5.0 |
| 2-612 | 0.109 | 0.316 | >5.0 |
| 2-613 | 0.036 | 0.171 | >5.0 |
| 2-614 | 0.02 | 0.081 | >5.0 |
| 2-615 | 0.359 | NT | NT |
| 2-616 | 0.970 | NT | NT |
| 2-617 | 0.036 | 0.08 | >5.0 |
| 2-618 | 0.049 | 0.074 | >5.0 |
| 2-619 | 0.038 | 0.067 | >5.0 |
| 2-620 | 0.844 | NT | NT |
| 2-621 | 0.295 | NT | NT |
| 2-622 | 0.112 | 0.143 | >5.0 |
| 2-623 | 0.035 | 0.104 | >5.0 |
| 2-624 | 0.022 | 0.063 | >5.0 |
| 2-625 | 0.015 | 0.046 | >5.0 |
| 2-626 | 0.456 | NT | NT |
| 2-627 | 0.041 | 0.084 | >5.0 |
| 2-628 | 1.86 | NT | NT |
| 2-629 | 0.304 | 0.633 | >5.0 |
| 2-630 | 0.266 | 0.465 | >5.0 |
| 2-631 | 0.661 | NI | NT |
| 2-632 | 0.017 | 0.073 | 4.16 |
| 2-633 | 0.029 | 0.069 | >5.0 |
| 2-634 | 0.033 | 0.189 | 3.66 |
| 2-635 | 0.428 | NT | NT |
| 2-636 | 0.123 | 0.392 | 3.13 |
| 2-637 | 0.038 | 0.16 | >5.0 |
| 2-638 | 0.017 | 0.021 | >5.0 |
| 2-639 | 2.25 | NT | NT |
| 2-640 | 0.763 | NT | NT |
| 2-641 | 0.052 | 0.153 | >5.0 |
| 2-642 | 0.044 | 0.133 | >5.0 |
| 2-644 | 0.194 | NT | NT |
| 2-645 | 0.053 | 0.225 | >5.0 |
| 2-646 | 0.025 | 0.052 | >5.0 |
| 2-647 | 0.052 | 0.074 | >5.0 |

NT = not tested

The results presented in Table 3 have been generated with the in vitro assays described above. These assays may be used to test any of the compounds described herein to assess and characterize a compound's biological activity.

Compounds showing activity in the coupled exchange assay are useful in the methods provided herein (see Section “METHODS OF USE”). See, e.g., Lanman et al., 2020. Hong et al., 2020. The inhibitory effect on tumor growth of the compounds provided herein can be shown, for example, using the following animal model.

Tumor cells are cultured, harvested and implanted subcutaneously into the right flank of female athymic nude mice. When tumors reach about 200 mm³, mice are randomized into treatment groups (n=10/group) and treatment is initiated (on days indicated on graphs). Tumor sizes and body weights are measured 2 to 3 times per week. Tumor volume is measured by digital calipers, calculated as L×W×H and expressed in mm³. Statistical significance of observed differences between growth curves can be evaluated by repeated measures analysis of covariance (RMANOVA) of the log transformed tumor volume data with Dunnett adjusted multiple comparisons comparing the control group to the treatment group. For combination studies. RMANOVA can be run with the combination group compared one to one with each single agent treatment group.

The foregoing description is provided for clearness of understanding only. No unnecessary limitations should be understood therefrom, as modifications within the scope of the invention may be apparent to those having ordinary skill in the art.

Throughout the specification, where compositions are described as including components or materials. it is contemplated that the compositions can also consist essentially of, or consist of: any combination of the recited components or materials. unless described otherwise. Likewise. where methods are described as including particular steps. it is contemplated that the methods can also consist essentially of, or consist of: any combination of the recited steps, unless described otherwise. The invention illustratively disclosed herein suitably may be practiced in the absence of any element or step which is not specifically disclosed herein.

All references, for example, a scientific publication, a patent, or a patent application publication, cited in this disclosure are incorporated herein by reference in their entirety and for all purposes to the same extent as if each reference was specifically and individually indicated to be incorporated by reference in its entirety for all purposes. In case of conflict between the present disclosure and incorporated references, the present disclosure should control.

What is claimed:

1. A compound of Formula (I): 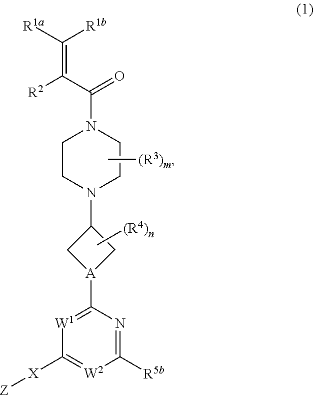
or a pharmaceutically acceptable salt thereof,
wherein:
m is 0, 1, 2, 3, or 4;
n is 0, 1, or 2;
A is N, CH, C-halo, C—CN, C—C₁₋₃ alkyl, C—C₁₋₃ haloalkyl, C—C₀₋₃ alkyleneOH, or C—C₀₋₃ alkylene-C₁₋₃ alkoxy;
each of W¹ and W² independently is N, CH, C-halo, C—CN, C—C₁₋₃ alkyl, C—C₂₋₃ alkenyl, C—C₂₋₃ alkynyl, C—C₁₋₃ haloalkyl, C—C₀₋₃ alkyleneOH, or C—C₀₋₃ alkylene-C₁₋₄ alkoxy, wherein each of the alkenyl and alkynyl is optionally substituted with 1-3 substituents and each substituent independently is halo, C₁₋₃ haloalkyl, C₀₋₃ alkyleneOH, or C₀₋₃ alkyleneC₁₋₄ alkoxy;
X is heterocycloalkyl or heterocycloalkenyl, each having 4-7 total ring atoms and 1-3 heteroatoms selected from N, O, and S, wherein each of the heterocycloalkyl and heterocycloalkenyl is optionally substituted with 1-3 substituents, and each substituent independently is halo, C₁₋₃ alkyl, C₁₋₃ haloalkyl, C₀₋₂ alkyleneOH, C₀₋₂ alkyleneC₁₋₃ alkoxy, or C₀₋₂ alkyleneCN;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S;
wherein each of the phenyl, heteroaryl, and bicyclic rings is optionally substituted with 1-4 substituents, and each substituent independently is halo, CN, C₁₋₆ alkyl, C₁₋₆ haloalkyl, C₂₋₆ alkenyl, C₂₋₆ haloalkenyl, C₀₋₆ alkylene-OH, C₀₋₆ alkylene-C₁₋₆ alkoxy, C₀₋₆ alkylene-N(R^{N1})₂, C₀₋₂ alkylene-cycloalkyl having 3-6 total ring atoms, C₀₋₂ alkylene-heterocycloalkyl having 3-6 total ring atoms and 1-3 heteroatoms selected from N, O, and S, or C₀₋₂ alkylene-phenyl,
wherein each of the C₁₋₆ alkyl, C₂₋₆ alkenyl, C₀₋₆ alkylene-C₁₋₃ alkoxy, cycloalkyl, heterocycloalkyl, and phenyl substituents is optionally substituted with 1-3 further substituents, and each further substituent independently is D, halo, C₁₋₃ alkyl, C₁₋₃ haloalkyl, C₁₋₂ alkyleneOH, C₁₋₂ alkylene-C₁₋₃ alkoxy, C₁₋₃ deuterated alkoxy, N(R^{N1})₂, (C═O)C₁₋₃ alkyl, cycloalkyl having 3-5 total atoms, heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S; or two adjacent further substituents, together with the atoms to which they are attached, form fused-cycloalkyl having 3-5 total ring atoms or fused-heterocycloalkyl having 3-5 total ring atoms and 1 or 2 heteroatoms selected from N, O, and S;
wherein each of the foregoing cycloalkyl and heterocycloalkyl further substituents is optionally substituted with 1 or 2 substituents, and each substituent independently is halo or C₁₋₃ alkyl,
each of R^{1a}, R^{1b}, and R² independently is H, D, halo, C₁₋₄ alkyl, C₁₋₄ haloalkyl, C₁₋₂ alkylene-OH, C₀₋₂ alkylene-C₁₋₄ alkoxy, C₀₋₂ alkylene-C₁₋₄ haloalkoxy, C₀₋₂ alkylene-CN, C₀₋₂ alkylene-N(R^{N1})₂, C₁₋₂ alkylene-heterocycloalkyl having 3-6 total ring atoms and 1 or 2 heteroatoms selected from N, O, and S, or R^{1b} and R², together with the carbon atoms to which they are attached, form 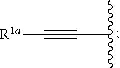
each R³ independently is C₁₋₃ alkyl, C₁₋₃ haloalkyl, 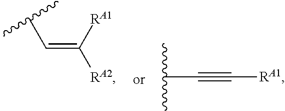
C₀₋₃ alkyleneCN, C₀₋₃ alkyleneOH, C₀₋₃ alkylene-C₁₋₃ alkoxy, oxo, spiro-cycloalkyl having 3-7 total ring atoms, spiro-cycloalkenyl having 4-7 total ring atoms, spiro-heterocycloalkyl having 3-7 total ring atoms and 1 or 2 heteroatoms selected from N, O and S, spiro-heterocycloalkenyl having 4-7 total ring atoms and 1 or 2 heteroatoms selected from N, O and S, or two adjacent R³, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1 or 2 heteroatoms selected from N, O, and S, or a fused heterocycloalkenyl ring having 4-7 total ring atoms and 1 or 2 heteroatoms selected from N, O, and S; or 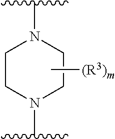
is deuterated:
each R⁴ independently is C₁₋₃ alkyl, C₁₋₃ haloalkyl, C₀₋₃ alkyleneCN, C₁₋₃ alkyleneOH, C₁₋₃ alkylene-C₁₋₃ alkoxy, oxo, spiro-cycloalkyl having 3-7 total ring atoms, or spiro-heterocycloalkyl having 4-7 total ring atoms and 1 or 2 heteroatoms selected from N, O and S;
R is C₁₋₃ haloalkyl, C₁₋₆ alkyl, C₂₋₄ alkenyl, C₂₋₄ alkynyl, halo, C₁₋₃ alkoxy, C₁₋₃ thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing independently is optionally substituted with 1-3 substituents, and each substituent independently is C₁₋₃ haloalkyl, C₀₋₆ alkylene-OH, C₀₋₆ alkylene-C₁₋₃ alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl;
each of R^{A1} and R^{A2} independently is H, C₁₋₃ alkyl, C₁₋₃ haloalkyl, or cycloalkyl having 3-5 total ring atoms; and
each R^{N1} independently is H or C₁₋₄ alkyl.

2. The compound or salt of claim 1, wherein at least one of R^{1a}, R^{1b}, and R² is H or D.

3. The compound or salt of claim 1 or 2, wherein each of R^{1a}, R^{1b}, and R² is H or D.

4. The compound or salt of claim 1 or 2, wherein two of R¹, R^{1b}, and R² are H and one of R^{1a}, R^{1b}, and R² is halo, C₁₋₄ alkyl, C₁₋₄ haloalkyl, C₁₋₂ alkylene-OH, C₀₋₂ alkylene-C₁₋₄ alkoxy, C₀₋₂ alkylene-C₁₋₄ haloalkoxy, C₀₋₂ alkylene-CN, C₀₋₂ alkylene-N(R^{N1})₂, or C₁₋₃ alkylene-heterocycloalkyl having 3-6 total ring atoms and 1 or 2 heteroatoms selected from N, O, and S.

5. The compound or salt of any one of claims 1, 2, and 4, wherein one of R^{1a}, R^{1b}, and R² is Br, Cl, F, CH₃, CH₂F, CHF₂, CF₃, CH₂OH, OCH₃, CH₂OCH₃, OCF₃, CH₂OCF₃, CN, CH₂CN, NH₂, N(CH₃)₂, CH₂NH₂, CH₂N(CH₃)₂, aziridin-1-yl-methyl, azetidin-1-yl-methyl, pyrrolidine-1-yl-methyl, piperidin-1-yl-methyl, or morpholin-1-yl-methyl.

6. The compound or salt of claim 1, wherein 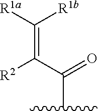
is

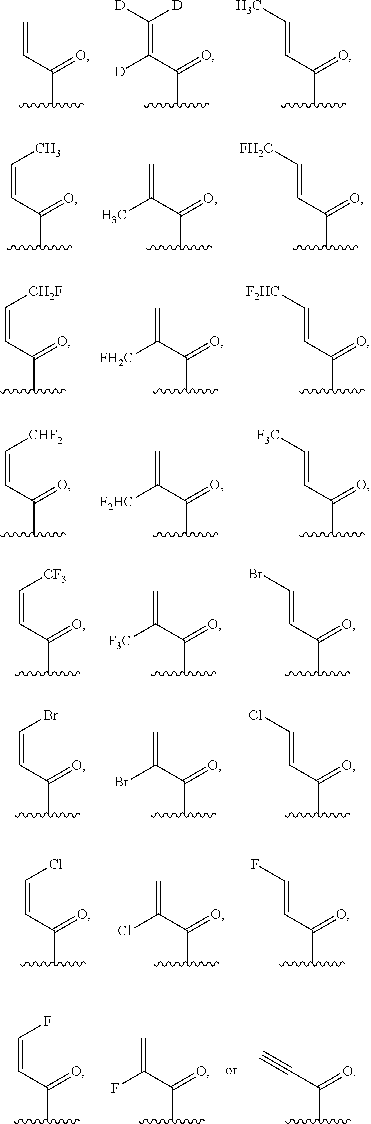

7. The compound or salt of any one of claims 1-6, wherein m is 0.

8. The compound or salt of any one of claims 1-6, wherein in is 1.

9. The compound or salt of any one of claims 1-6, wherein m is 2.

10. The compound or salt of claim 8 or 9, w herein each R³ independently is CH₃, CH₂CH₃, CF₃, CHF₂, CH₂F, 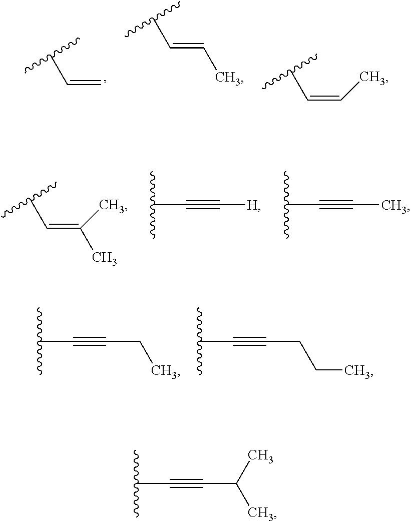
CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl, or two adjacent R³, together with the atoms to which they are attached, form a fused cyclopropyl ring or a fused cyclobutyl ring.

11. The compound or salt of any one of claims 1-6, wherein in is 0; or m is 1 and R³ is CH₃, CF₃, CHF₂, CH₂F, CN, CH₂CN, CH₂OH, CH₂OCH₃, or spiro-oxetanyl.

12. The compound or salt of claim 1, wherein 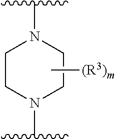
is

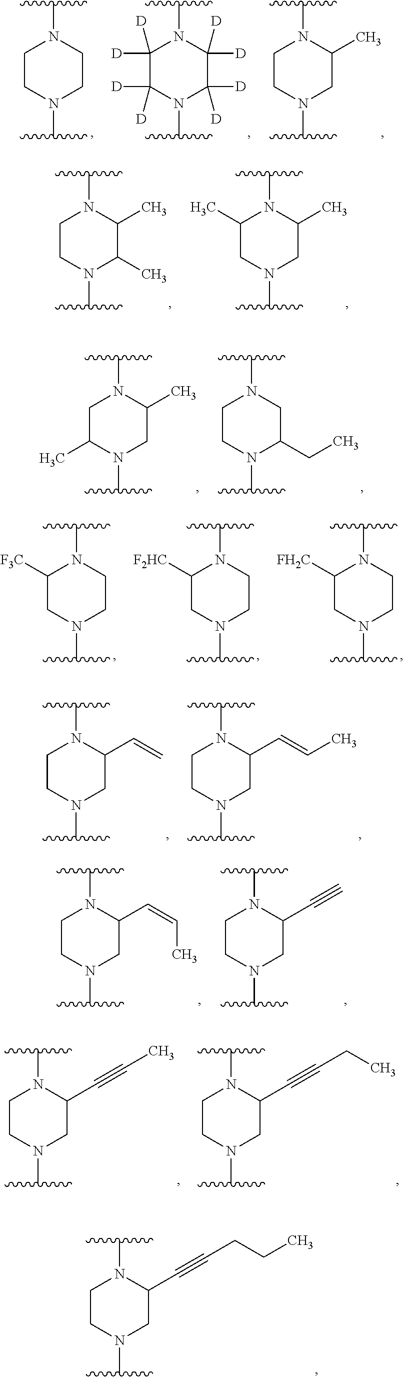

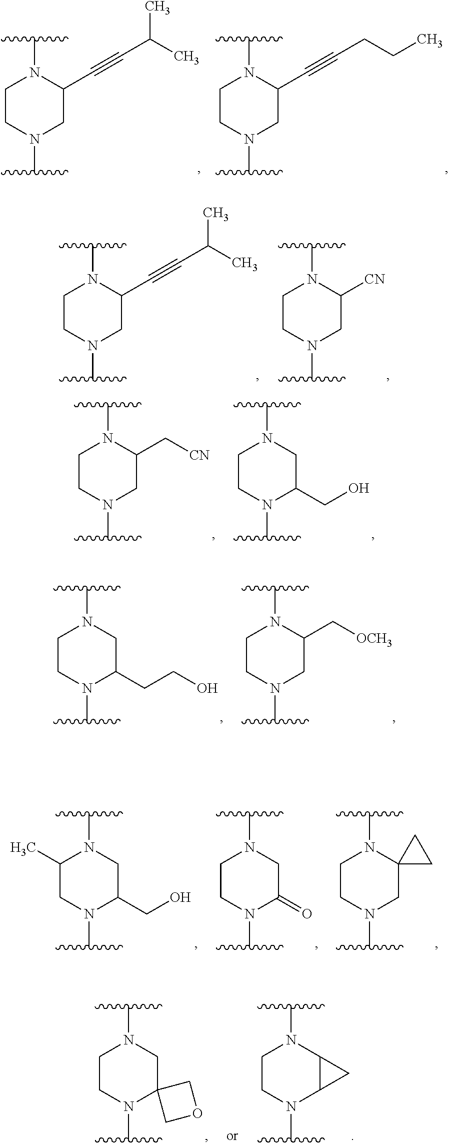

13. The compound or salt of claim 12, wherein 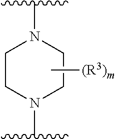
is

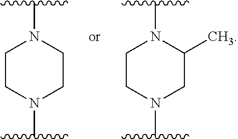 [SMILES: *N1CCN(*)CC1 ; *N1CCN(*)C(C)C1]

14. The compound or salt of any one of claims 1-13, wherein A is N.

15. The compound or salt of any one of claims 1-13, wherein A is CH, C—F, C—Cl, C—CN, C—CH₃, C—CH₂F, C—CHF₂, C—CF₃, C—OH, C—CH₂OH, C—OCH₃, or C—CH₂OCH₃.

16. The compound or salt of any one of claims 1-15, wherein n is 0.

17. The compound or salt of any one of claims 1-15, wherein n is 1.

18. The compound or salt of any one of claims 1-15, wherein n is 2.

19. The compound or salt of claim 17 or 18, wherein each R⁴ independently is CH₃, CH₂CH₃, CH₂CH₂CH, CH(CH₃)₂, CF₃, CHF₃, CH₂F, CN, CH₂CN, CH₂OH, CH₂CH₂OH, CH₂OCH₃, CH₂CH₂OCH₃, oxo, spiro-cyclopropyl, spiro-cyclobutyl, or spiro-oxetanyl.

20. The compound or salt of any one of claims 1-13, wherein 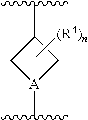
is

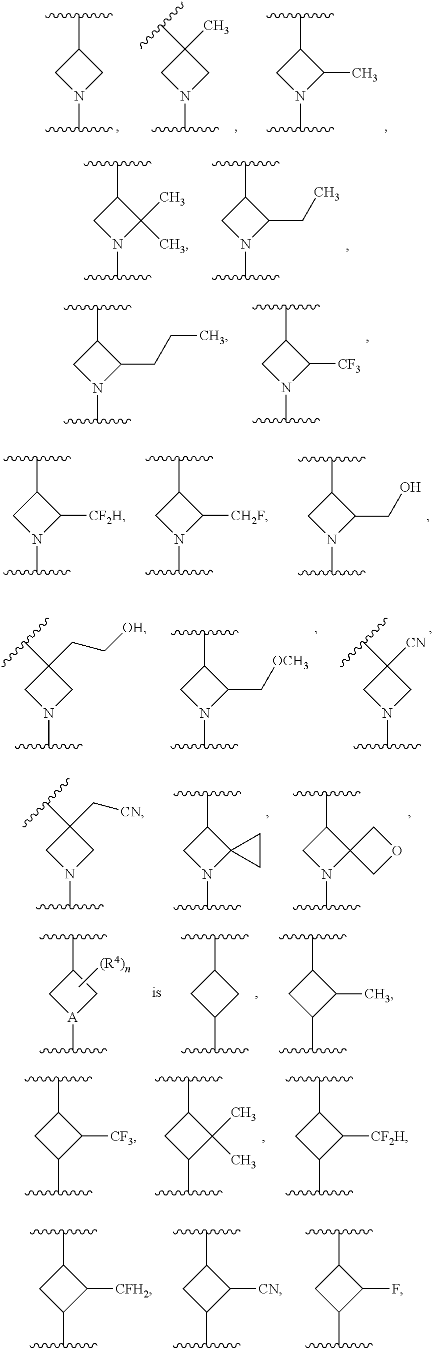

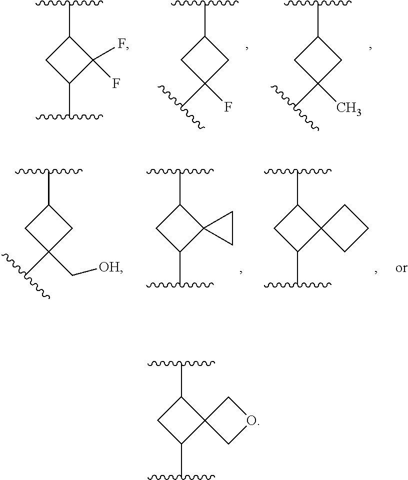

21. The compound or salt of claim 20, wherein 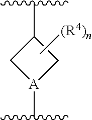
is

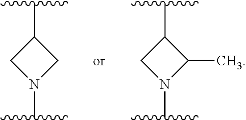 [SMILES: *C1CN(*)C1 ; *C1CN(*)C1C]

22. The compound or salt of any one of claims 1-21, wherein W¹ is N.

23. The compound or salt of any one of claims 1-21, wherein W¹ is CH.

24. The compound or salt of any one of claims 1-21, wherein W¹ is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C—CH₂OH, C—OCH₃, or C—CH₂OCH₃.

25. The compound or salt of any one of claims 1-24, wherein W² is N.

26. The compound or salt of any one of claims 1-24, wherein W² is CH.

27. The compound or salt of any one of claims 1-24, wherein W² is C—F, C—Cl, C—CN, C—CHN, C—CH₂CH₃, C—CH₂F, C—CHF₂, C—CF₃, C—CH═CH₂, C—C(OH)═CH₂, C—CH═CH(OH), C—CCH, C—OH, C—CH₂OH, C—OCH₃, or C—CH₂OCH₃.

28. The compound or salt of any one of claims 1-21, wherein W¹ is CH and W² is N.

29. The compound or salt of any one of claims 1-28, wherein R⁵ is C₁₋₃ haloalkyl.

30. The compound or salt of claim 29, wherein R⁵ is CF₃, CF₂H, CFH₂.

31. The compound or salt of any one of claims 1-28, wherein R⁵ is CH₃, CH₂CH₃, CH₂CH₂CH₃, CH(CH₃)₂, CH═CH₂, CH═CHCH₃, 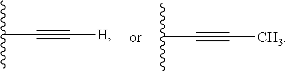
wherein each of the foregoing is optionally substituted with 1-3 substituents, and each substituent independently is C₁₋₃ haloalkyl, C₀₋₆ alkylene-OH, C₀₋₆ alkylene-C₁₋₃ alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl.

32. The compound or salt of claim 31, wherein each substituent independently is CH₃, CF₃, CF₂H, CFH₂, OH, OCH₃, OCF₃, CH₂OH, CH₂OCH₃, cyclopropyl, cyclobutyl, or phenyl.

33. The compound or salt of any one of claims 1-28, wherein R⁵ is Br, Cl, F, OCH₃, SCH₃, CH₃, CH₂CH₃, CH₂CH₂CH₃, CH(CH₃)₂,

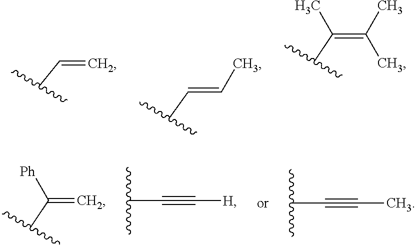

34. The compound or salt of any one of claims 1-21, wherein W¹ is CH, W² is N, and R⁵ is CF₃, CF₂H, or CFH₂.

35. The compound or salt of any one of claims 1-21, wherein 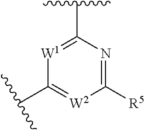
is

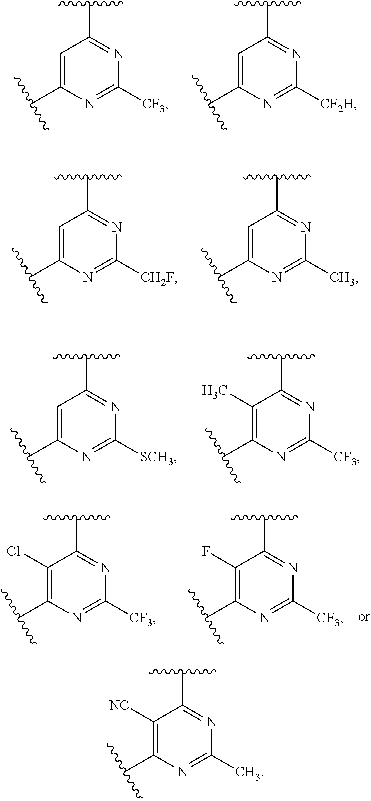

36. The compound or salt of any one of claims 1-35, wherein:
X is 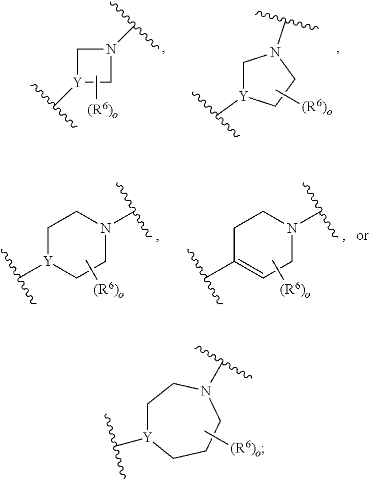
Y is N, C—H, C-halo, C—CN, C—C₁₋₃ alkyl, C—C₁₋₃ haloalkyl, C—C₀₋₃ alkyleneOH, or C—C₀₋₃ alkylene-C₁₋₄ alkoxy;
o is 0, 1, 2, 3, or 4; and
each R⁶ independently is halo, CN, C₁₋₃ alkyl, C₂₋₃ alkenyl, C₁₋₃ haloalkyl, C₀₋₃ alkylene-OH, C₀₋₃ alkylene-C₁₋₃ alkoxy, deuterated C₀₋₃ alkylene-C₁₋₃ alkoxy, C₁₋₄ alkylene-N(R^{N1})₂, oxo, ═CH₂, spiro-cycloalkyl having 3-7 total atoms, spiro-cycloalkenyl having 4-7 total atoms, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 4-7 total ring atoms and 1-3 heteroatoms selected from N, O, and S, or a fused heterocycloalkenyl ring having 4-7 total ring atoms and 1 or 2 heteroatoms selected from N, O, and S; or two non-adjacent R⁶ join together to form a C₁₋₃ alkylene bridge, a C₂₋₃ alkenylene bridge, a C₁₋₃ ether bridge, or a C₁₋₃ thioether bridge; or Y and an adjacent R⁶, together with the atoms to which they are attached, form a fused cycloalkyl ring having 3-7 total ring atoms, a fused cycloalkenyl ring having 4-7 total ring atoms, a fused heterocycloalkyl ring having 3-7 total ring atoms and 1-3 heteroatoms selected from N, O, and S, or a fused heterocycloalkenyl ring having 4-7 total ring atoms and 1 or 2 heteroatoms selected from N, O, and S; wherein the cycloalkyl, cycloalkenyl, heterocycloalkyl or heterocycloalkenyl of any of the foregoing is optionally substituted with 1-4 substituents, and each substituent independently is halo, C₁₋₃ alkyl, C₁₋₃ haloalkyl, C₀₋₂ alkyleneOH, C₀₋₂ alkyleneC₁₋₃ alkoxy, or C₀₋₂ alkyleneCN; and
each R^{N1} independently is H or C₁₋₄ alkyl.

37. The compound or salt of claim 36, wherein X is

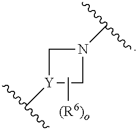

38. The compound or salt of claim 36, wherein X is

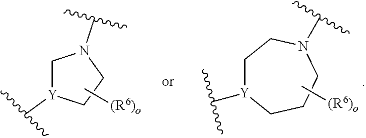

39. The compound or salt of claim 36, wherein X is

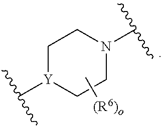

40. The compound or salt of claim 36, wherein X is

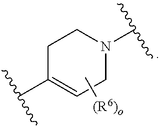

41. The compound or salt of any one of claims 36-39, wherein Y is N.

42. The compound or salt of any one of claims 36-39, wherein Y is CH.

43. The compound or salt of claim 36-39, wherein Y is C—F, C—Cl, C—CH₃, C—CH₂CH₃, C—CH₂F, C—CHF₂, C—CF₃, C—OH, C—CH₂OH, C—OCH₃, or C—CH₂OCH₃.

44. The compound or salt of any one of claims 36-43, wherein o is 0.

45. The compound or salt of claim 36-43, wherein o is 1.

46. The compound or salt of claim 36-43, wherein o is 2.

47. The compound or salt of any one of claims 36-43 and 45-46, wherein each R⁶ independently is Br, Cl, F, CN, CH₃, CH₂F, CHF₂, CF₃, OH, CH₂OH, OCH₃, OCD₃, CH₂OCH₃, CH₂N(CH₃)₂, oxo, ═CH₂, spiro-cyclopropyl, spirocyclobutyl, spiro-oxetanyl, or spiro-tetrahydrofuranyl, or two adjacent R⁶, together with the atoms to which they are attached, form fused-cyclopropyl, fused-cyclobutyl, or fused-cyclopentyl, and any of the foregoing spiro and fused rings is optionally substituted with 1 or 2 substituents, and each substituent independently is halo, C₁₋₃ alkyl, C₁₋₃ haloalkyl, C₀₋₂ alkyleneOH, C₀₋₂ alkyleneC₁₋₃ alkoxy, or C₀₋₂ alkyleneCN.

48. The compound or salt of claim 47, wherein each substituent independently is F, Cl, OH, OCH₃, OCH₂CH, or CN.

49. The compound or salt of any one of claims 36-43 and 46, wherein two non-adjacent R⁶ join together to form a C₁₋₃ alkylene bridge, a C₂₋₃ alkenylene bridge, a C₁₋₃ ether bridge, or a C₁₋₃ thioether bridge.

50. The compound or salt of claim 49, wherein two non-adjacent R⁶ join together to form —CH₂—, —CH₂CH₂—, —CH₂CH₂CH₂—, —CH₂—CH═CH— or —CH₂OCH₂—.

51. The compound or salt of any one of claims 1-36, wherein X is

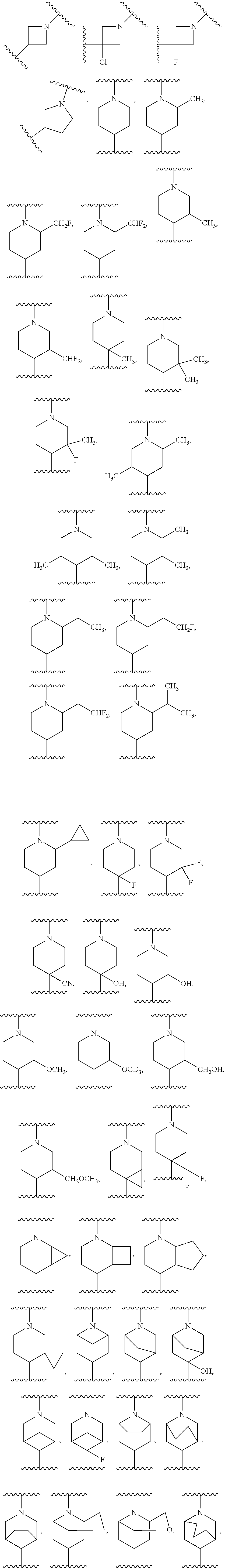

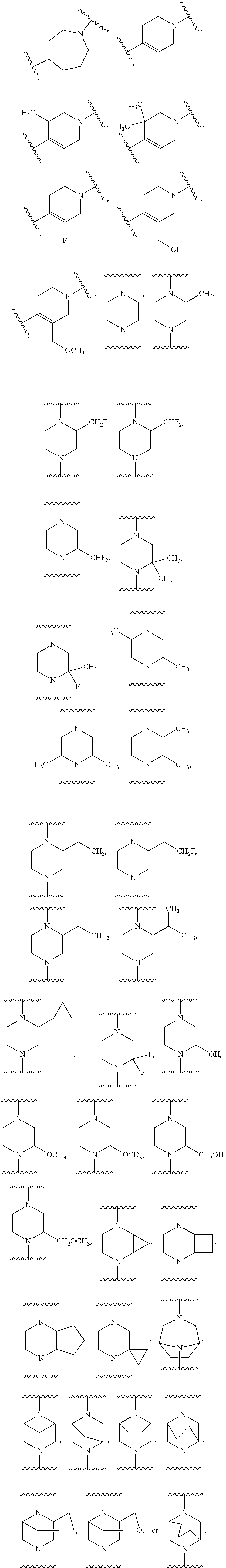

52. The compound or salt of claim 51, wherein X is

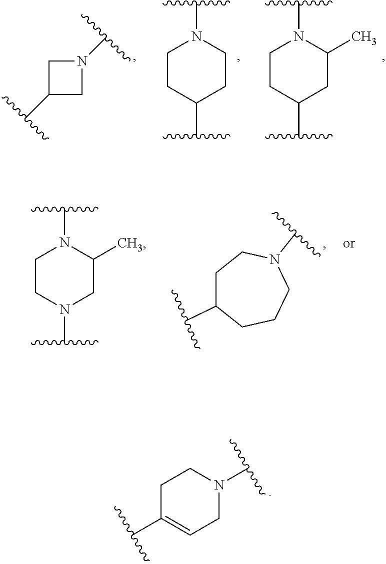

53. The compound or salt of any one of claims 1-52, wherein Z is phenyl that is optionally substituted with 1-4 substituents, and each substituent independently is halo, C₀₋₃ alkyleneCN, C₀₋₃ alkyleneOH, C₀₋₃ alkylene-C₁₋₄ alkoxy, C₀₋₃ alkylene-C₁₋₄ thioalkoxy, or 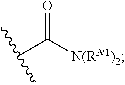
and each R^{N1} independently H or CH₃.

54. The compound or salt of claim 53, wherein each substituent independently is F, Cl, CN, OCH₃, SCH₃, CH₂OH, or

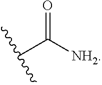 [SMILES: *C(N)=O]

55. The compound or salt of claim 53 or 54, wherein Z is

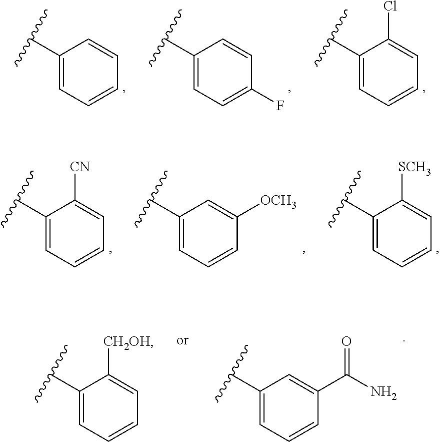

56. The compound or salt of any one of claims 1-52, w herein Z is heteroaryl comprising 5 or 6 total ring atoms and 1-3 heteroatoms selected from N, O, and S, wherein the heteroaryl is optionally substituted with 14 substituents, and each substituent independently is halo, CN, C₁₋₆ alkyl, C₁₋₆ haloalkyl, C₂₋₆ alkenyl, C₂₋₆ haloalkenyl, C₀₋₆ alkylene-OH, C₀₋₆ alkylene-C₁₋₃ alkoxy, C₀₋₆ alkylene-N(R^{N1})₂, C₀₋₂ alkylene-cycloalkyl having 3-6 total ring atoms, C₀₋₂ alkylene-heterocycloalkyl having 3-6 total ring atoms and 1-3 heteroatoms selected from N, O, and S, or C₀₋₂ alkylene-phenyl,
wherein each of the C₁₋₆ alkyl, C₂₋₆ alkenyl, C₀₋₆ alkylene-C₁₋₃ alkoxy, cycloalkyl, heterocycloalkyl, and phenyl substituents is optionally substituted with 1-3 further substituents and each further substituent independently is D, halo, C₁₋₃ alkyl, C₁₋₆ haloalkyl, C₁₋₂ alkyleneOH, C₁₋₂ alkylene-C₁₋₃ alkoxy, C₁₋₃ deuterated alkoxy, N(R^{N1})₂, (C═O)C₁₋₃ alkyl, cycloalkyl having 3-5 total ring atoms, heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S; or
two adjacent further substituents, together with the atoms to which they are attached, form fused-cycloalkyl having 3-5 total ring atoms or fused-heterocycloalkyl having 3-5 total ring atoms and 1 or 2 heteroatoms selected from N, O, and S;
wherein each of the foregoing cycloalkyl and heterocycloalkyl further substituents is optionally substituted with 1 or 2 substituents, and each substituent independently is halo or C₁₋₃ alkyl; and
each R^{N1} independently is H or C₁₋₃ alkyl.

57. The compound or salt of claim 56, wherein Z is optionally substituted: pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl.

58. The compound or salt of claim 57, wherein Z is optionally substituted: pyrazolyl or pyridyl.

59. The compound or salt of any one of claims 56-58, wherein the heteroaryl is substituted with 1 or 2 substituents.

60. The compound or salt of any one of claims 56-59, wherein each substituent independently is Br, Cl, F, CN, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, NH₂, CH═NH₂, CH₂NHCH₃, CH₂N(CH₃)₂, CH₂CH₂NH₂, CH₂CH₂NHCH₃, CH₂CH₂N(CH₃)₂, C₁₋₆ alkyl selected from CH₃, CH₂CH₃, CH₂CH₂CH₃, and CH(CH₃)₂, C₂₋₆ alkenyl selected from CH═CH₂, CH₂CH═CH₂, and CH═CHCH₃, C₀₋₆ alkylene-C₁₋₃ alkoxy selected from OCH₃, CH₂OCH₃, CH₂CH₂OCH₃, CH₃CH₂OCH₂CH, CH₂CH₂CH₂OCH₃, CH(CH₃)OCH₃, CH(CH₃)CH₂OCH₃, CH(OCH₃)CH₂OCH₃, CH(CH₃)(OCH₃)CH₂OCH₃, C(CH₃)₂OCH₃, C(CH₃)₂CH₂OCH₃, CH₂CH(CH₃)OCH₃, CH₂(CH₃)(OCH₃)OCH₃, CH₂C(CH₃)OCH₃, and CH₂C(CH₃)₂OCH₃, cycloalkyl selected from cyclopropyl, cyclobutyl, cyclopentyl, and cyclohexyl, or heterocycloalkyl selected from azetidinyl, pyrrolidinyl, piperidinyl, pyrazolidinyl, oxetanyl, tetrahydrofuranyl, tetrahydropyranyl, isoxazolidinyl, and morpholinyl;
wherein each of the C₁₋₆ alkyl, C₂₋₆ alkenyl, C₀₋₆ alkylene-C₁₋₃ alkoxy, cycloalkyl, and heterocycloalkyl substituents independently is optionally substituted with 1-3 further substituents and each further substituent independently is D, halo, C₁₋₃ alkyl, C₁₋₃ haloalkyl, C₁₋₂ alkyleneOH, C₁₋₂ alkylene-C₁₋₃ alkoxy, C₁₋₃ deuterated alkoxy, N(R^{N1})₂, (C═O)C₁₋₃ alkyl, cycloalkyl having 3-5 total ring atoms, heterocycloalkyl having 3-5 total ring atoms and 1 or 2 heteroatoms selected from N, O, and S, spiro-cycloalkyl having 3-5 total ring atoms, or spiro-heterocycloalkyl having 3-5 total ring atoms and 1 or 2 heteroatoms selected from N, O, and S; or two adjacent further substituents, together with the atoms to which they are attached, form fused-cycloalkyl having 3-5 total ring atoms or fused-heterocycloalkyl having 3-5 total ring atoms and 1 or 2 heteroatoms selected from N, O, and S.

61. The compound or salt of any one of claims 56-60, wherein each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, azetidinyl, spiro-oxetanyl or spiro-azetidinyl; wherein each of the foregoing oxetanyl, azetidinyl, spiro-oxetanyl, and spiro-azetidinyl is optionally substituted with F, CH₃, or a combination thereof.

62. The compound or salt of claim 61, wherein each further substituent independently is D, Br, Cl, F, OH, CH₃, CF₃, CF₂H, CFH₂, OCH₃, CH₂OCH₂OCD₃, N(CH₃)₂, (C═O)CH₃,

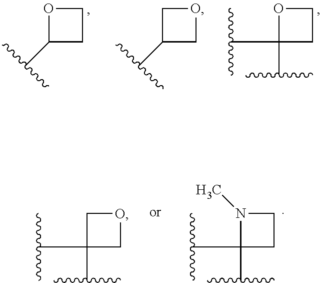

63. The compound or salt of any one of claims 56-62, wherein each substituent independently is Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃, CH₂CH₂CH₂OCH₃, CH₂CH₂CH₂OCD₃, CH(CH₃)OCH₃, CH(CH₃)CH₂OCH₃, CH(OCH₃)CH₂OCH₃, CH(CH₃)(OCH₃)CH₂OCH₃, CH(CH₂F)(CH₃)CH₂OCD₃, CH(CH₃)CH₂OCD₃, C(CH₃)₂OCH₃, C(CH₃), CH₂OCH₃, C(CH₃)₂CH₂OCD₃, CH₂CH(CH₃)OCH₃, CH₂(CH₃)(OCH₁)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

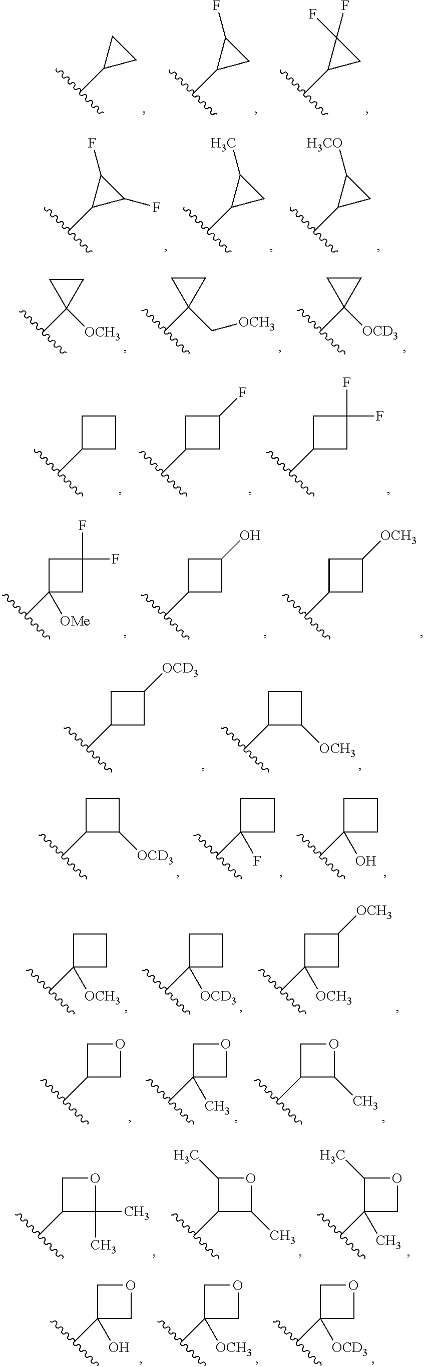

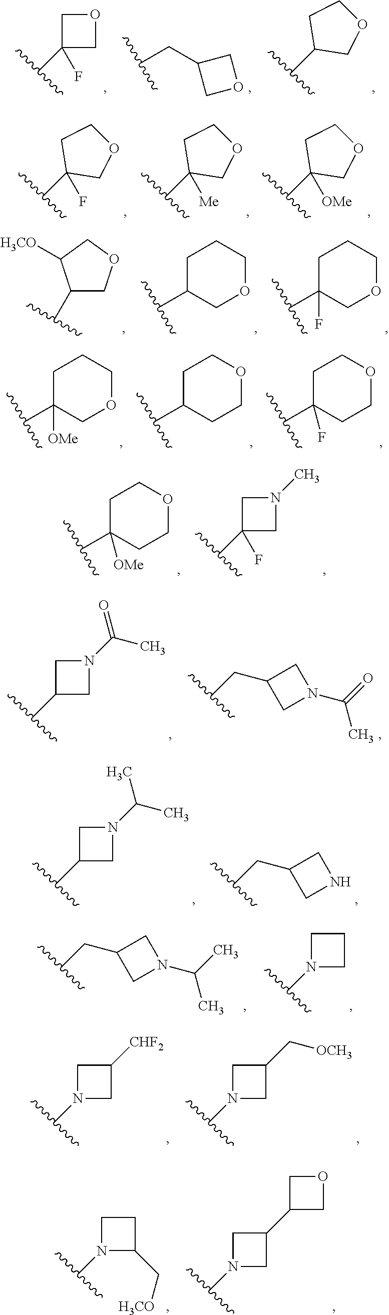

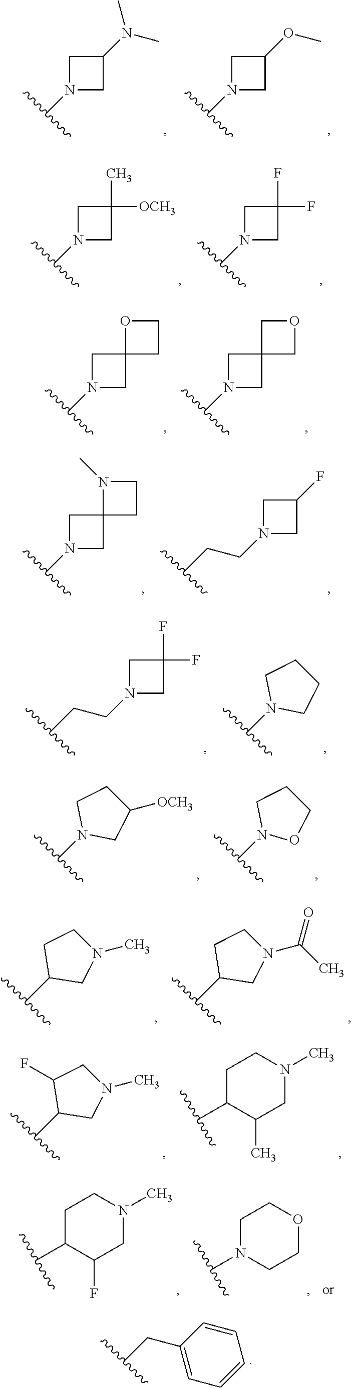

64. The compound or salt of claim 63, wherein each substituent independently is CH₃, CH(CH₃)₂, C(CH₃)₂OH, CH₂OCD₃, C(CH₃)₂CH₂OH, CH₂CH₂OCH₃, CF₂CH₂OCH₃, CH₂CH₂OCD₃, CH(CH₃)OCH₃, CH₂CH₂CH₂OCH₃, CH₂CH(CH₃)OCH₃, CH₂C(CH₃)₂OCH₃,

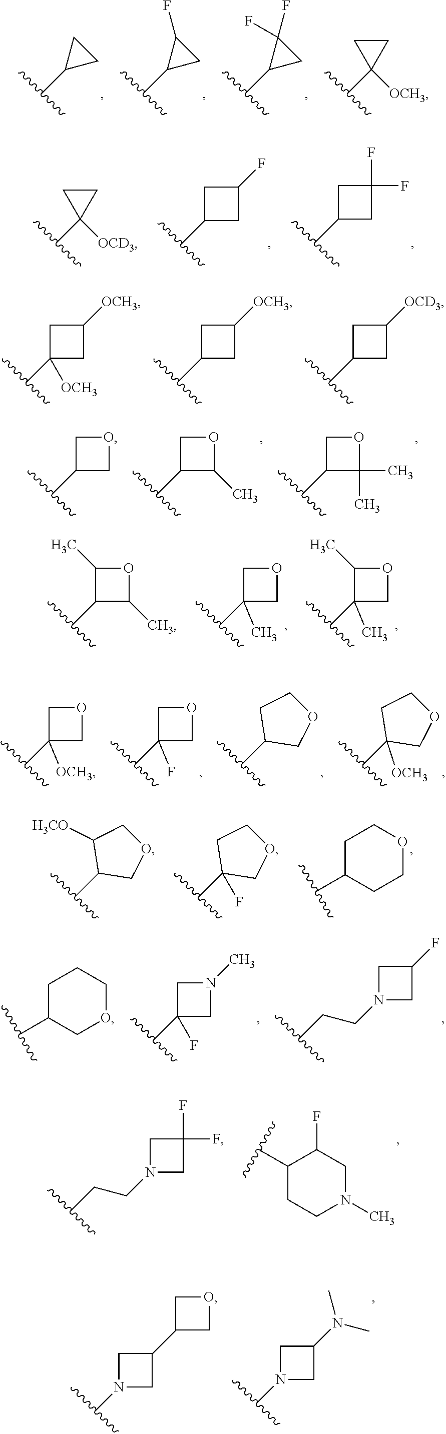

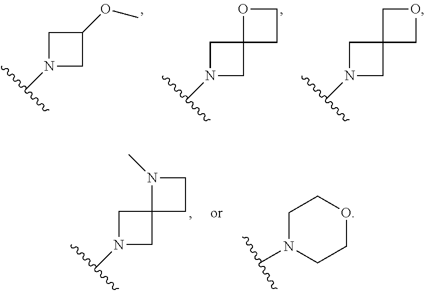

65. The compound or salt of any one of claims 1-52, wherein Z is

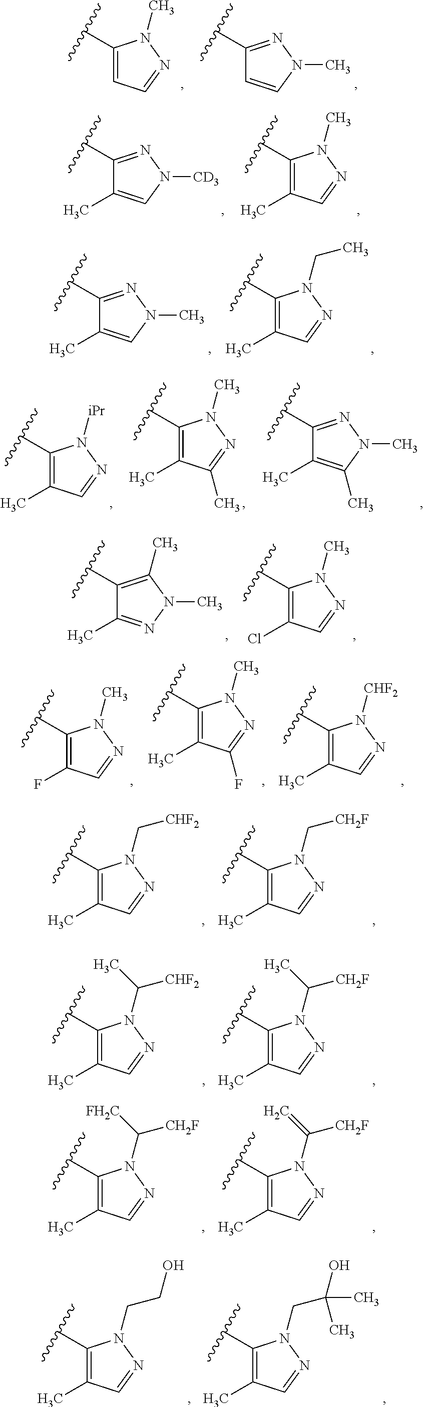

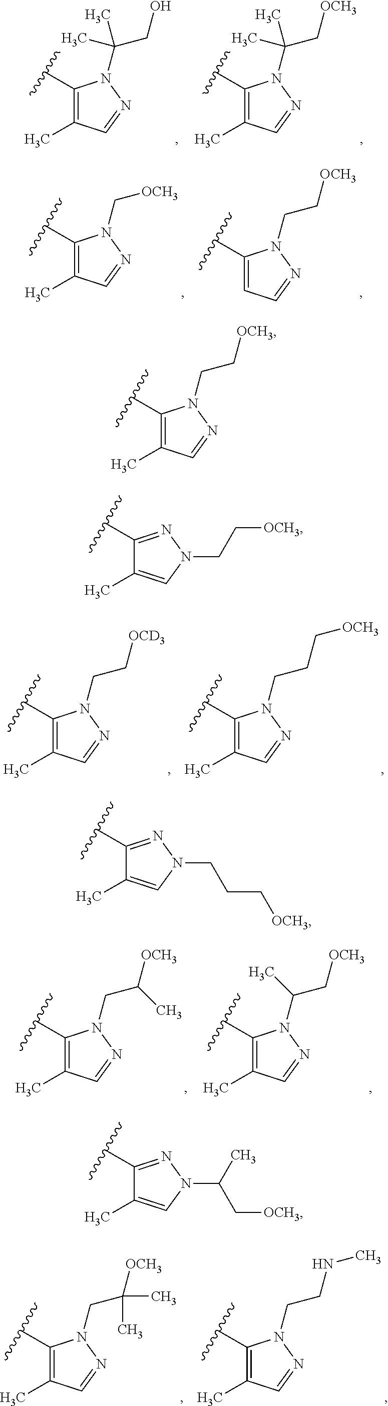

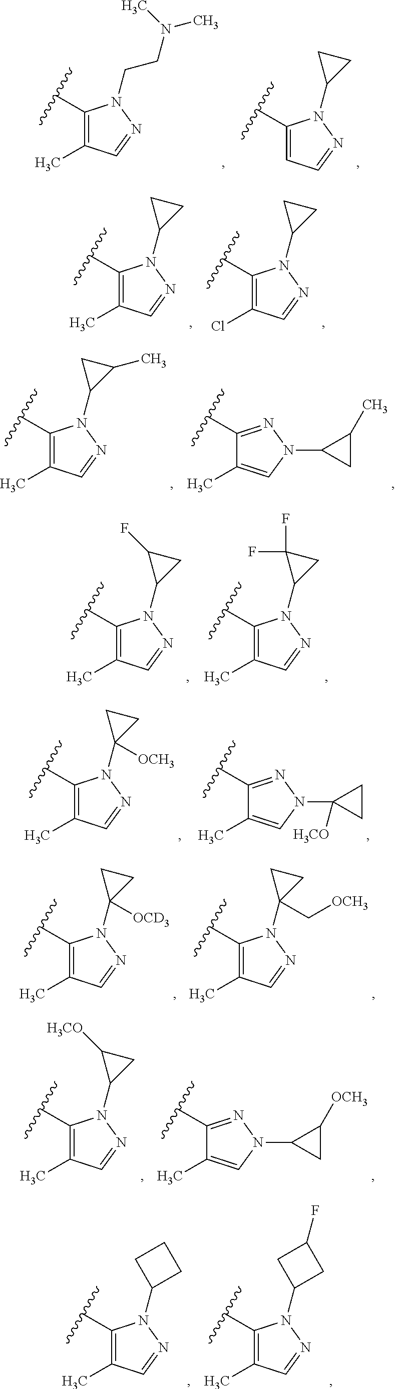

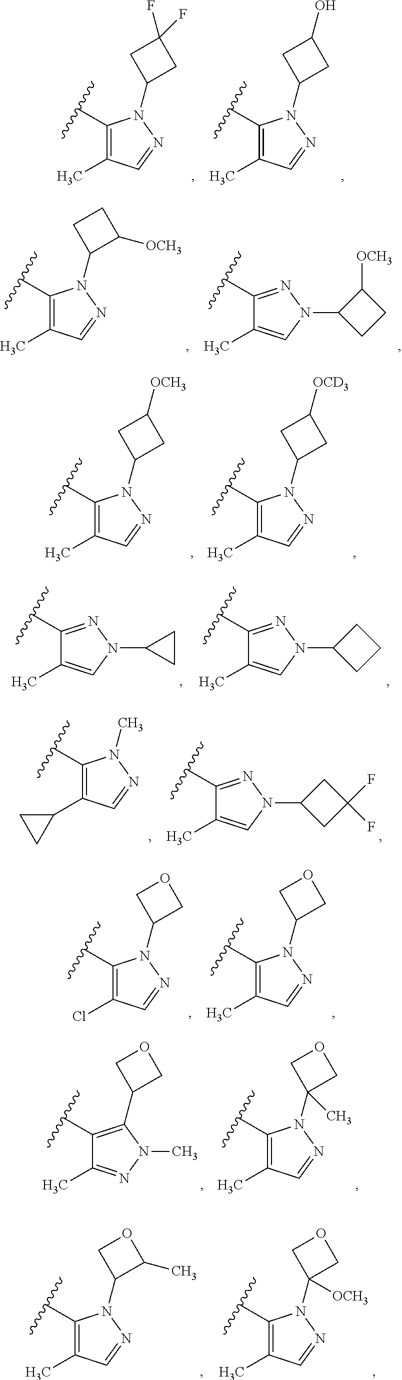

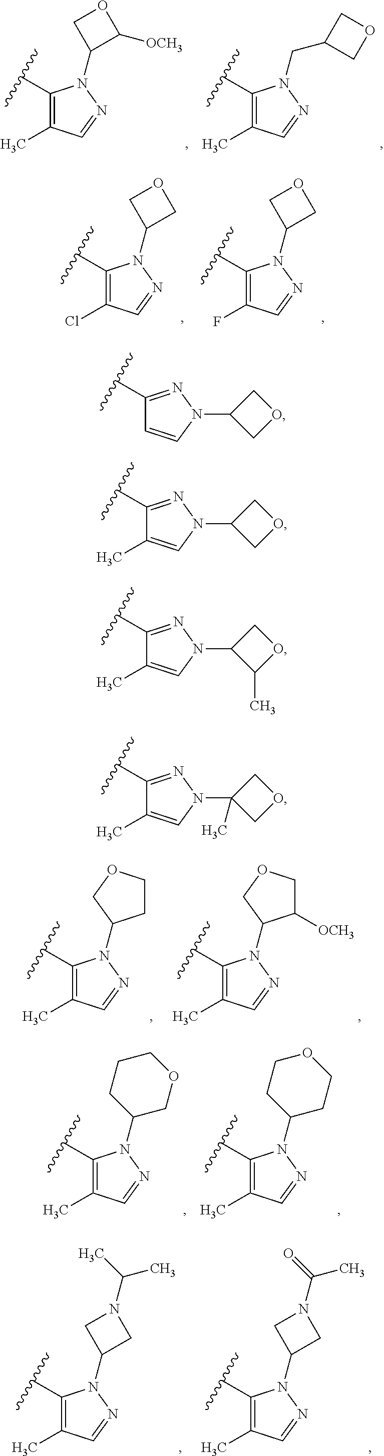

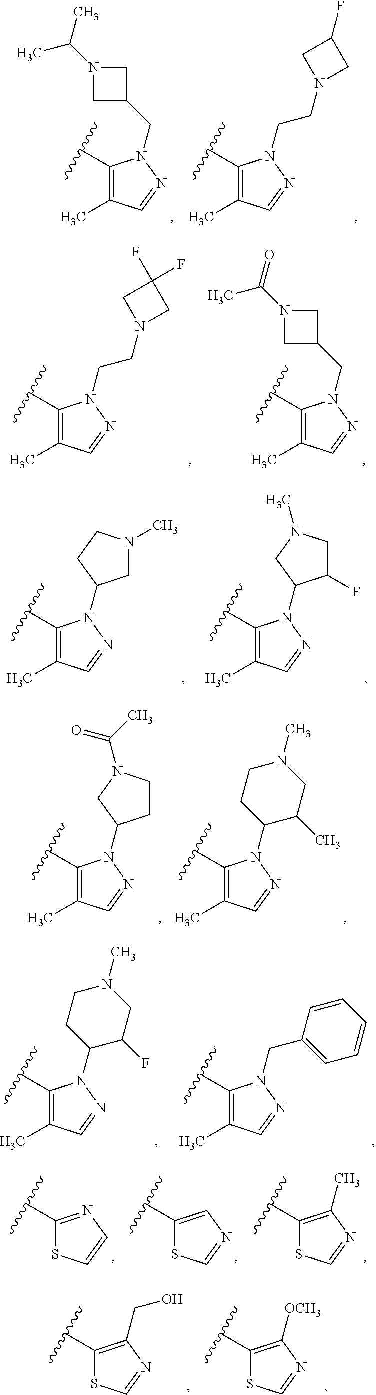

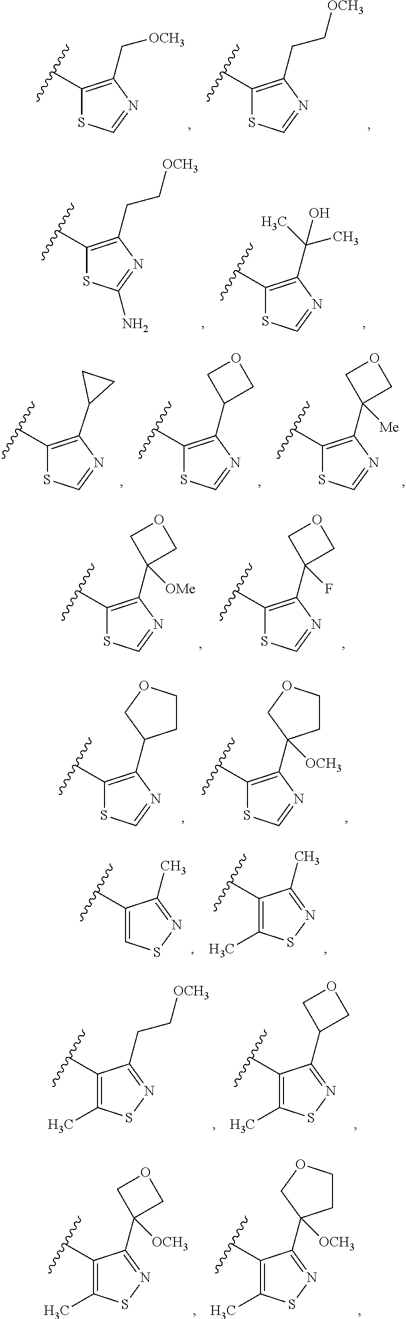

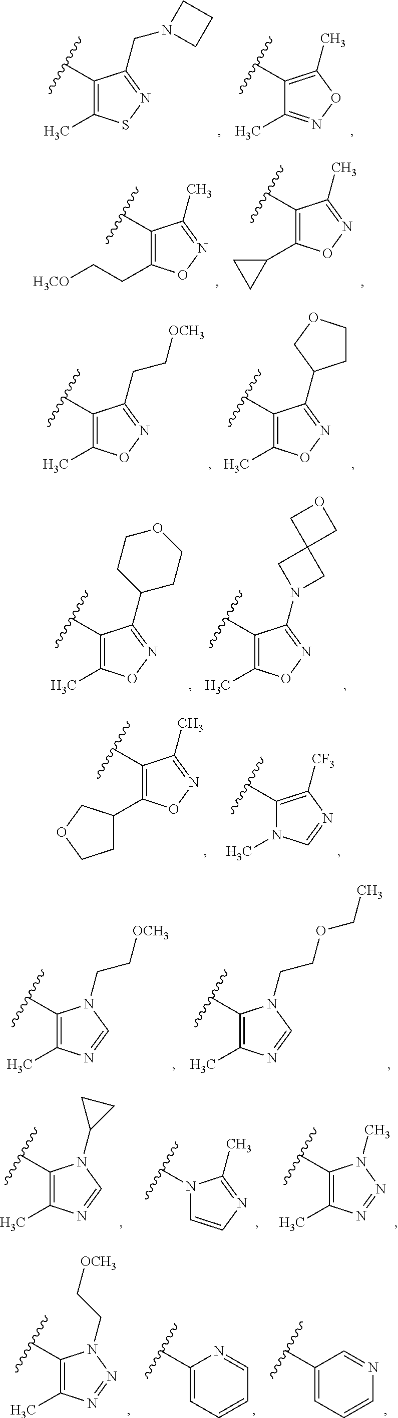

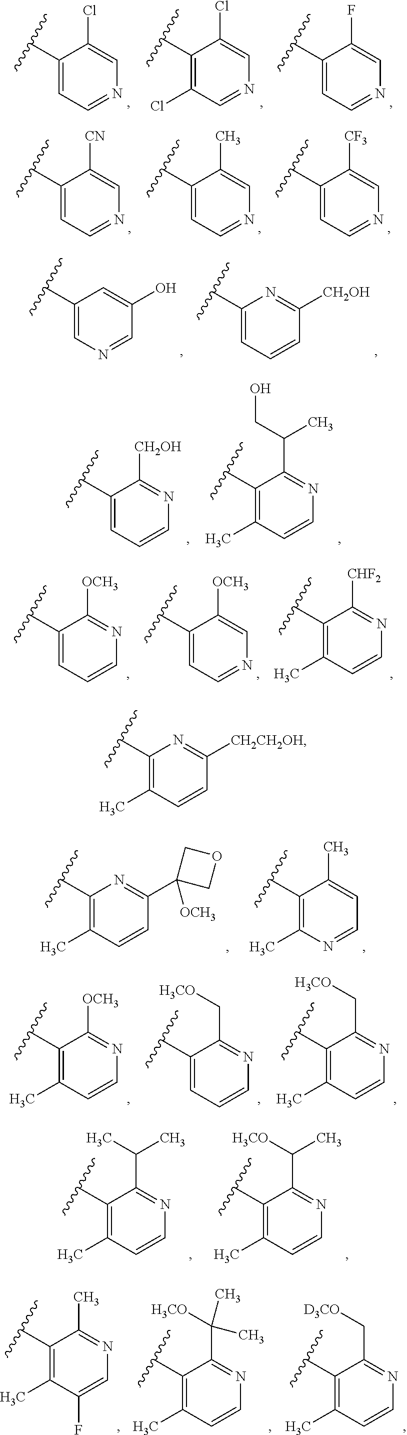

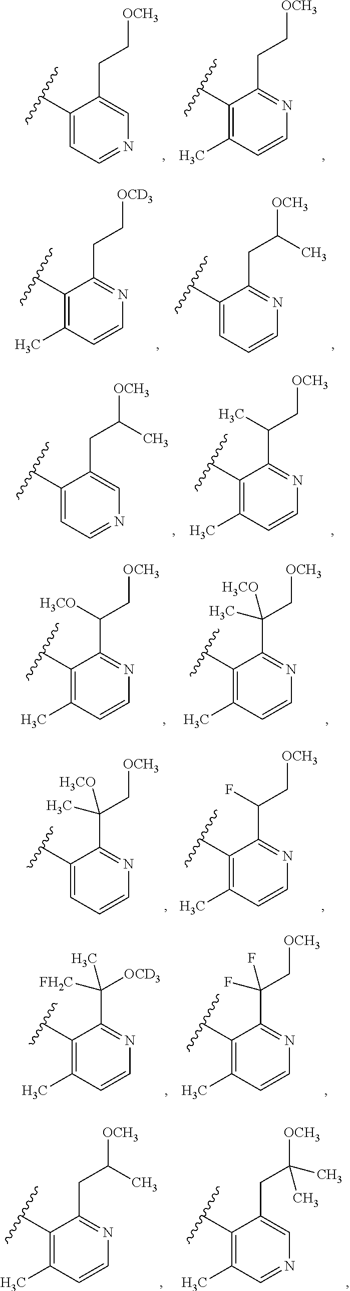

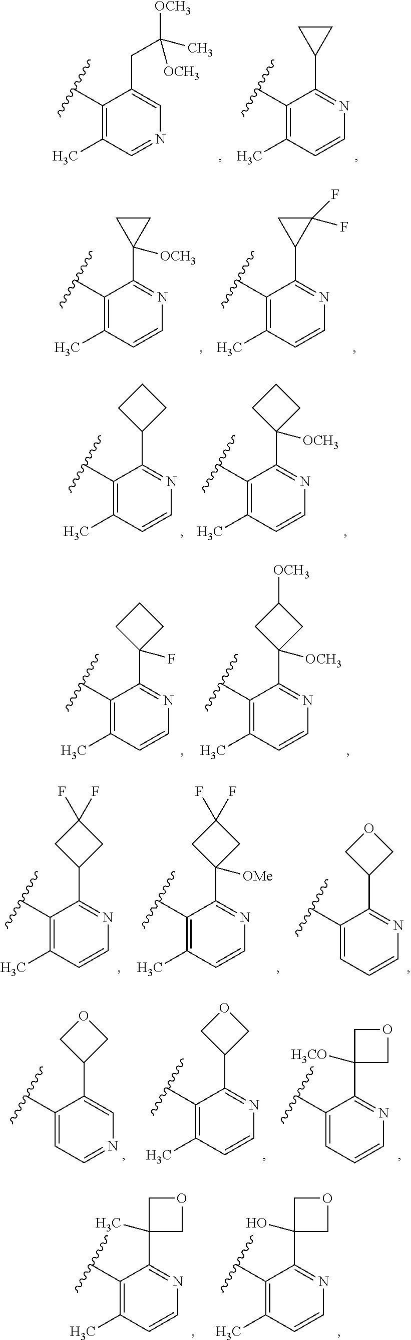

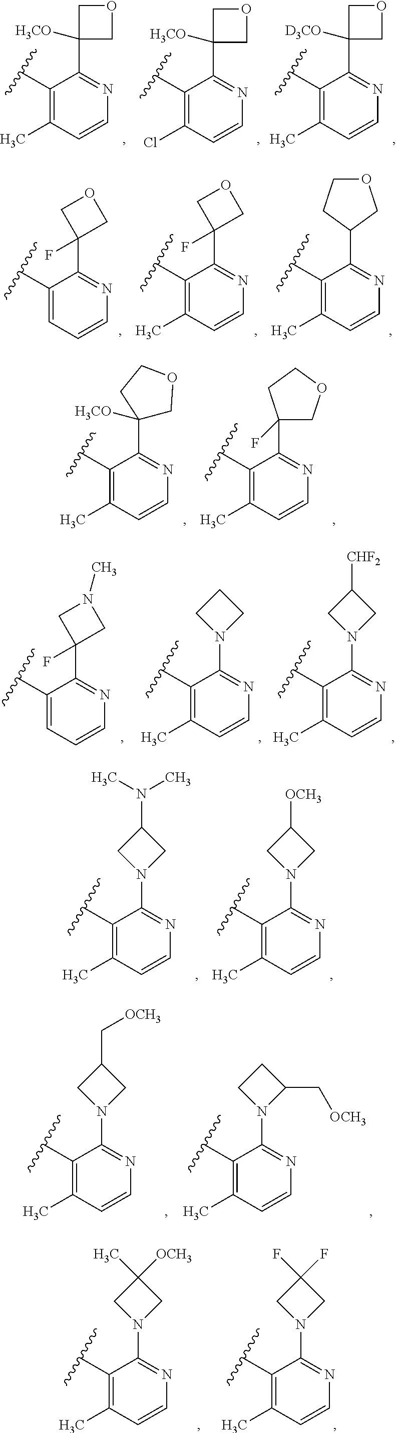

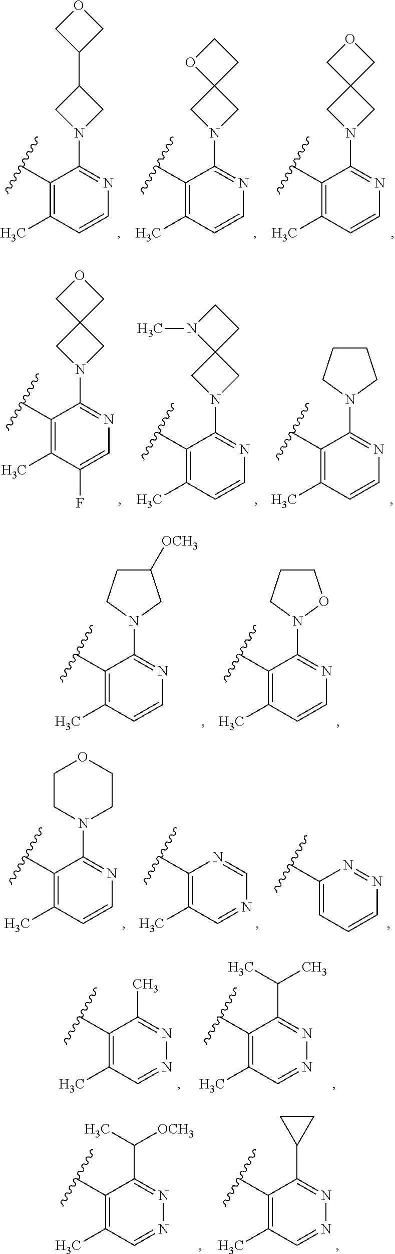

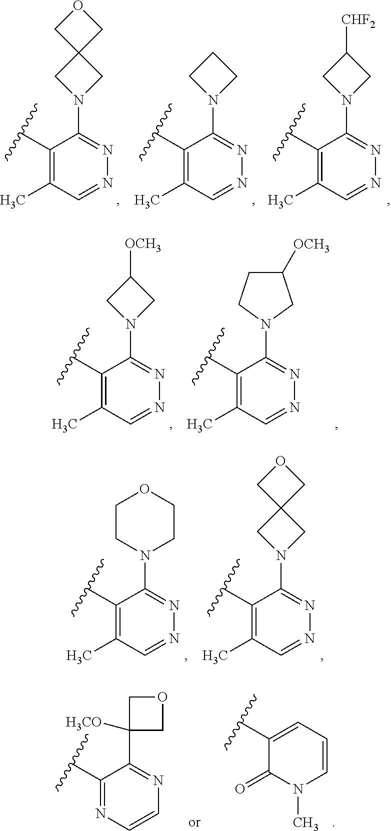

66. The compound or salt of any one of claim 65, wherein Z is

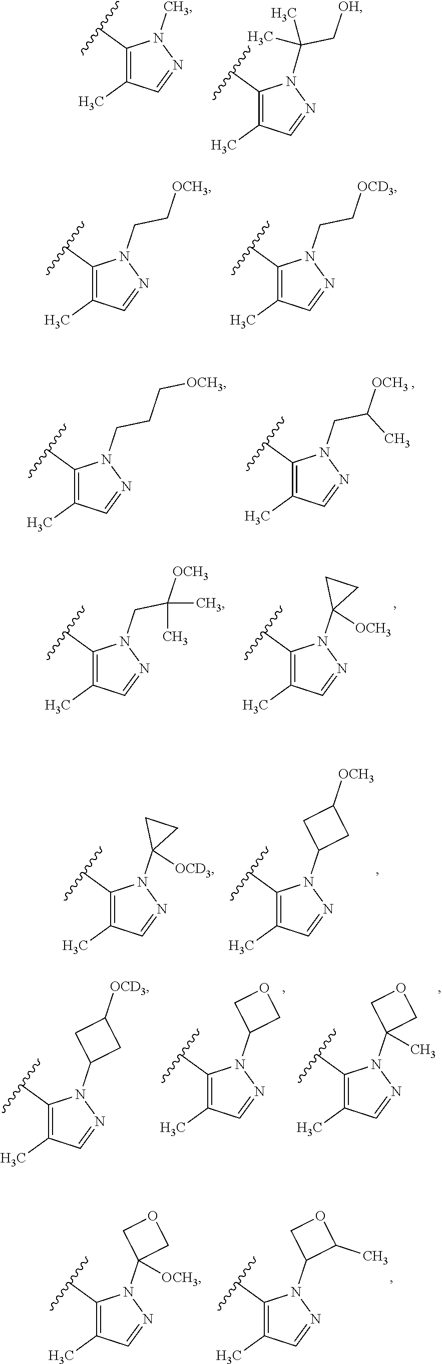

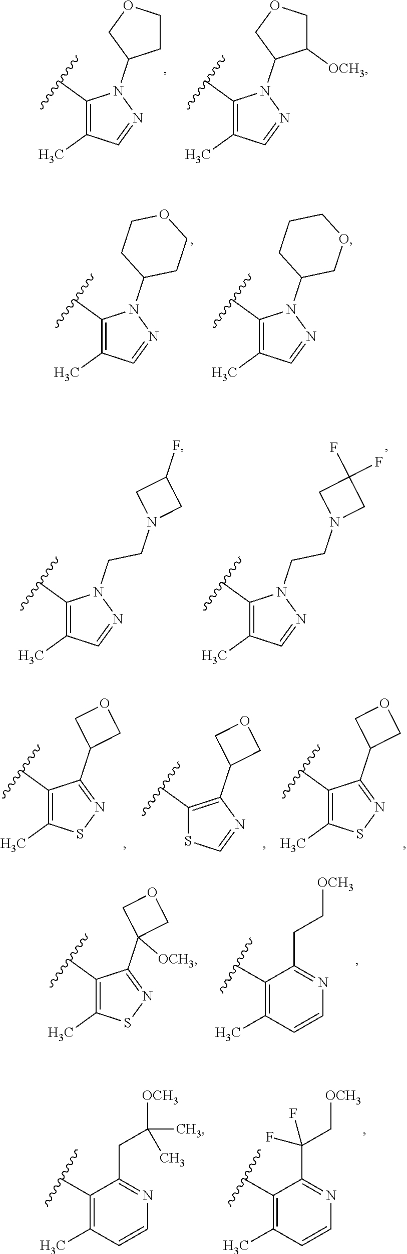

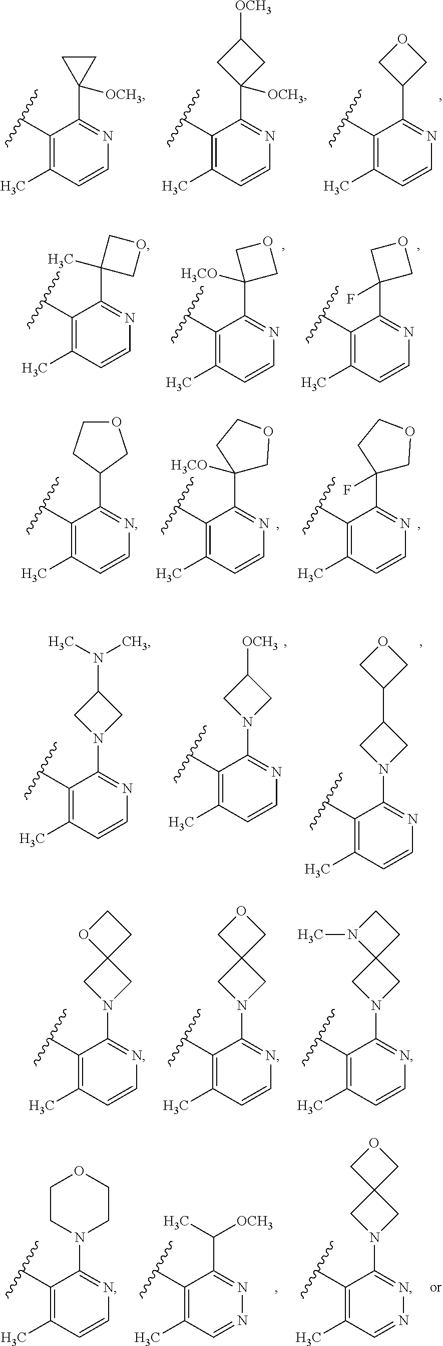

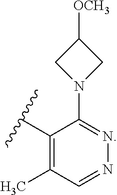 [SMILES: *c1c(C)cnnc1N1CC(OC)C1]

67. The compound or salt of claim 66, wherein Z is

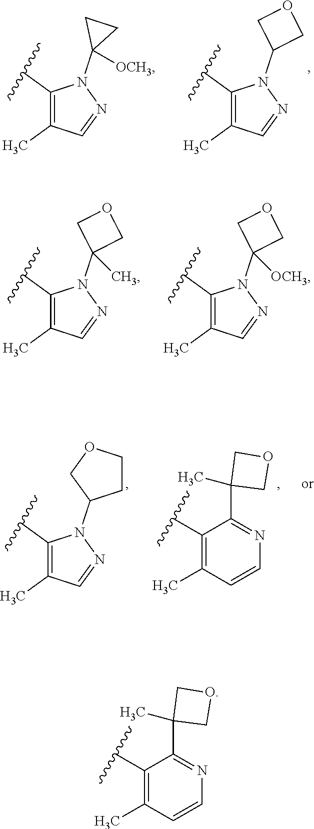

68. The compound or salt of any one of claims 1-52, wherein Z is a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein the bicyclic ring is optionally substituted with 1-4 substituents, and each substituent independently is halo, CN, C₁₋₆ alkyl, C₁₋₆ haloalkyl, C₀₋₆ alkylene-OH, or C₀₋₆ alkylene-C₁₋₃ alkoxy.

69. The compound or salt of claim 68, wherein Z is

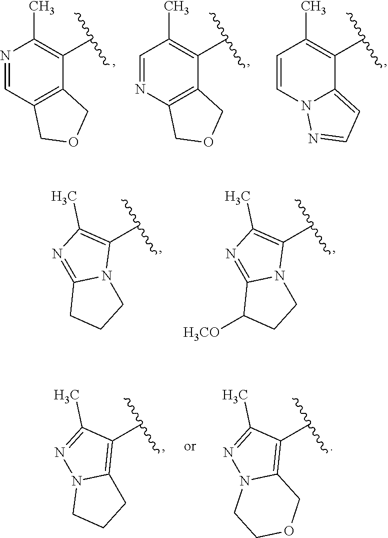

70. The compound or salt of claim 1, wherein: 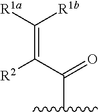
is 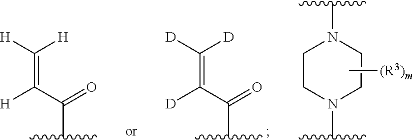
is 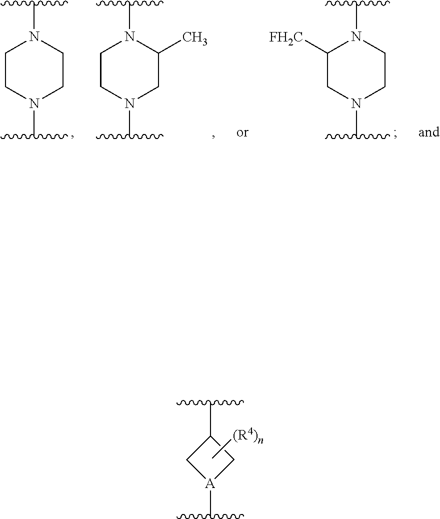
is

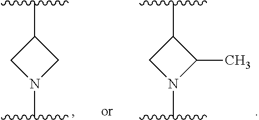

71. The compound of claim 70, wherein 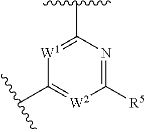
is

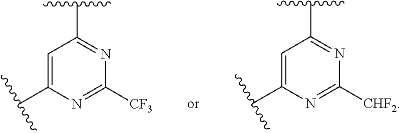 [SMILES: *c1cc(*)nc(C(F)(F)F)n1 ; *c1cc(*)nc(C(F)F)n1 ; "or"]

72. The compound or salt of claim 70 or 71, wherein X is

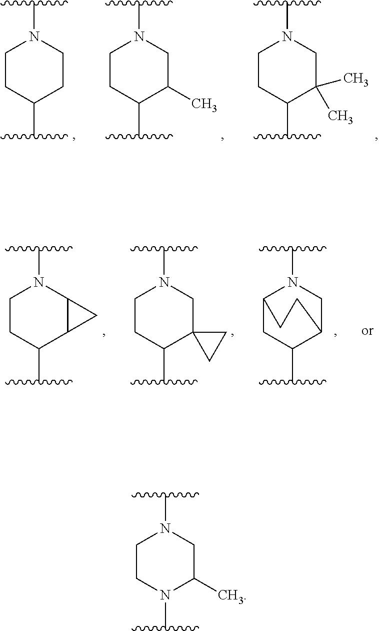

73. The compound or salt of any one of claims 70-72, wherein Z is

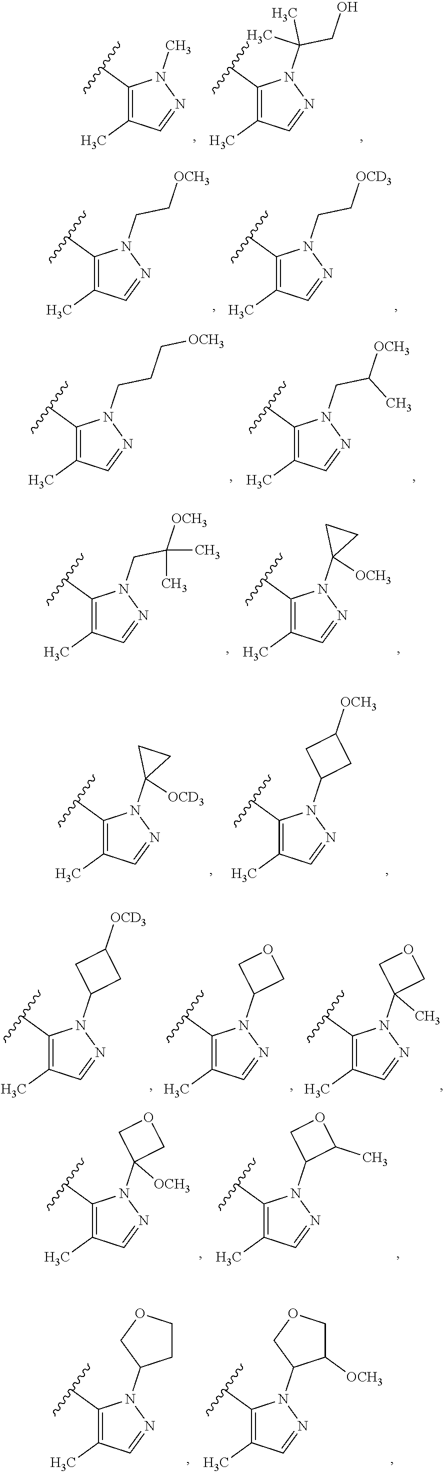

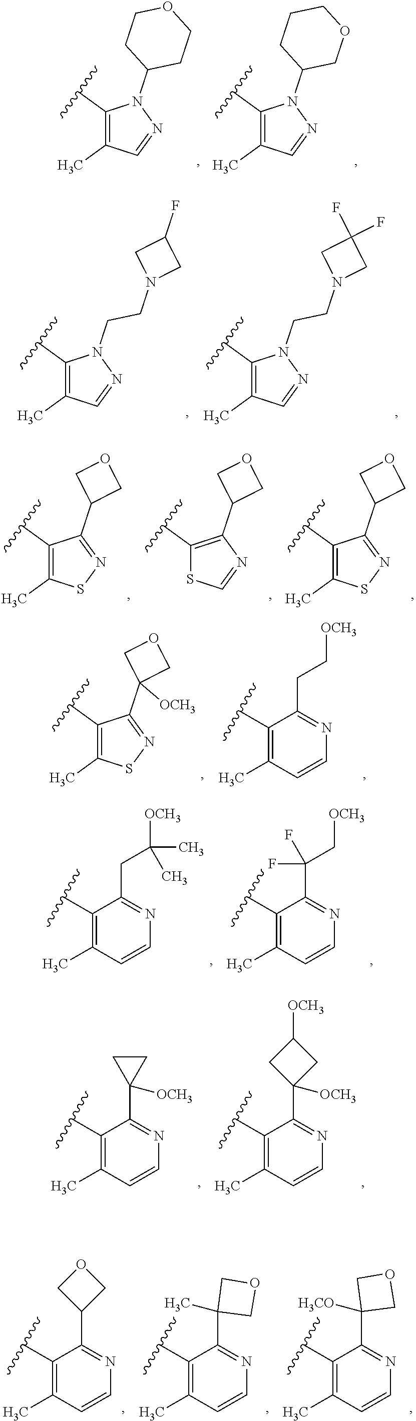

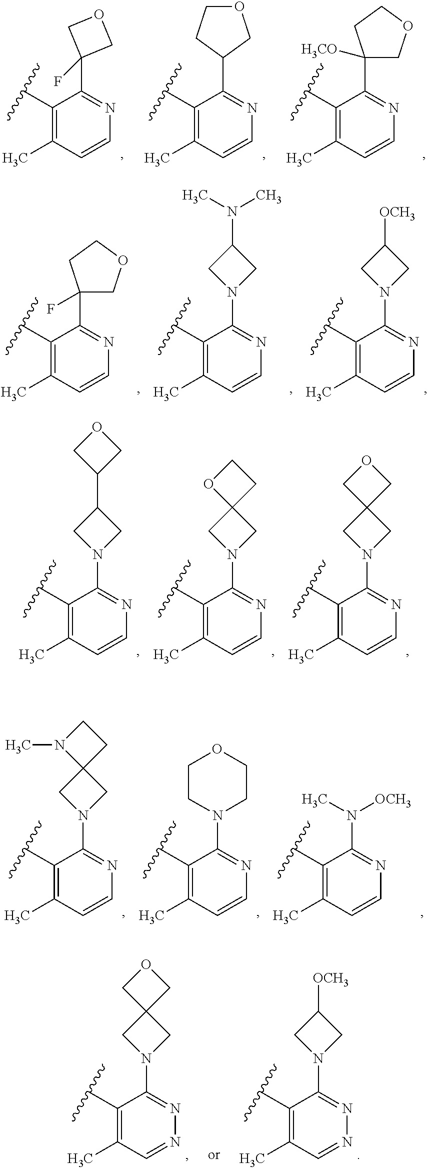

74. The compound or salt of claim 1, wherein the compound is a compound of Formula (I′): 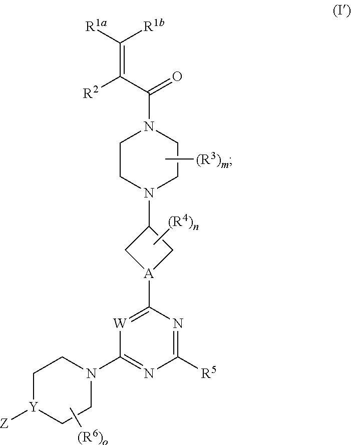
Formula (IA): 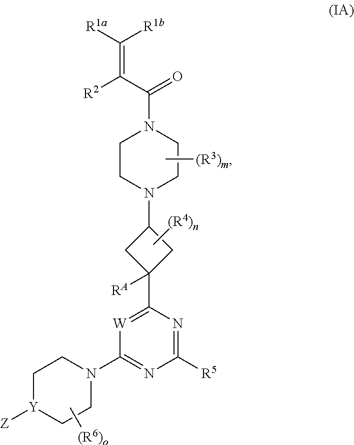
wherein R^{A} is H, halo, CN, C₁₋₃ alkyl, C₁₋₃ haloalkyl, C₀₋₃ alkyleneOH, or C₀₋₃ alkylene-C₁₋₄ alkoxy; Formula (IB): 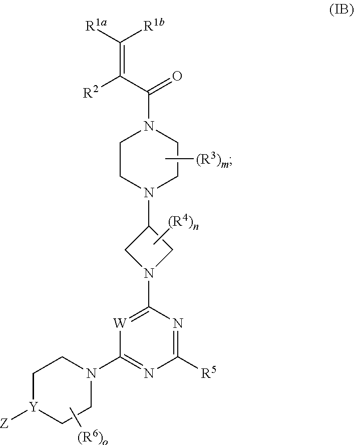
Formula (IC): 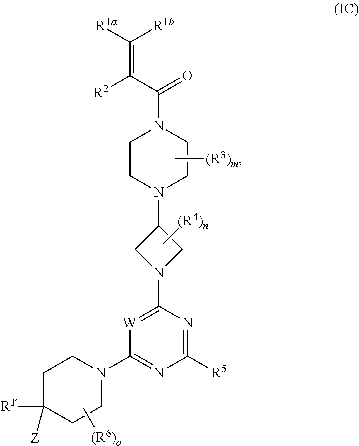
wherein R^{Y} is H, halo, CN, C₁₋₃ alkyl, C₁₋₃ haloalkyl, C₀₋₃ alkyleneOH, or C₀₋₃ alkylene-C₁₋₄ alkoxy; Formula (ID): 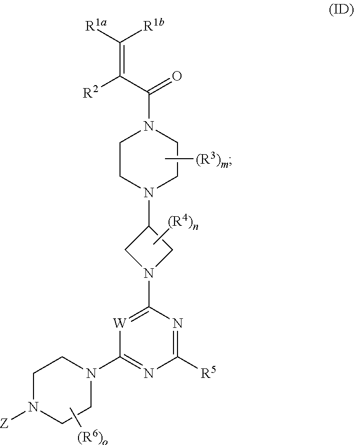
Formula (IE): 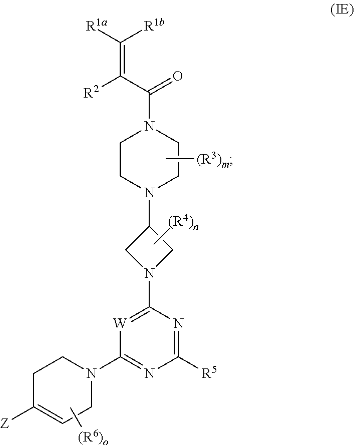
or Formula (IF): 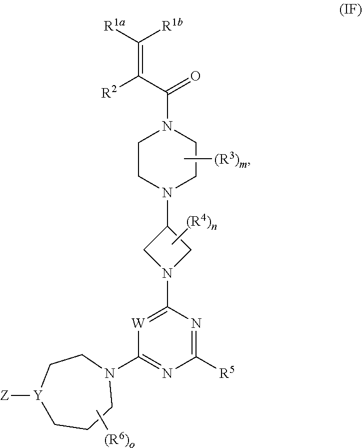
or a pharmaceutically acceptable salt of any of the foregoing.

75. The compound of claim 1, wherein the compound is a compound listed in Table A, or a pharmaceutically acceptable salt thereof.

76. The compound of claim 1, wherein the compound is a compound listed in Table E, or a pharmaceutically acceptable salt thereof.

77. A pharmaceutical composition comprising the compound or salt of any one of claims 1-76 and a pharmaceutically acceptable excipient.

78. The compound or salt of any one of claims 1-76, or the pharmaceutical composition of claim 77 for use as a medicament.

79. The compound or salt of any one of claims 1-76 or the pharmaceutical composition of claim 77 for use in treating cancer.

80. The compound or salt of any one of claims 1-76 or the pharmaceutical composition of claim 77 for use in treating cancer, wherein one or more cancer cells express KRAS G/2C mutant protein.

81. The compound or salt of claim 79 or 80, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

82. Use of a compound or salt of any one of claims 1-76 or the pharmaceutical composition of claim 77 in the preparation of a medicament for treating cancer.

83. Use of a compound or salt of any one of claims 1-76 or the pharmaceutical composition of claim 77 in the preparation of a medicament for treating cancer, wherein one or more cancer cells express KRASG12C mutant protein.

84. The use of claim 82 or 83, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

85. A method of treating cancer in a subject in need thereof, the method comprising administering to the subject a therapeutically effective amount of the compound or salt of any one of claims 1-76, or the pharmaceutical composition of claim 77.

86. The method of claim 85, wherein one or more of the cancer cells express KRAS G12C mutant protein.

87. The method of claim 85 or 86, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

88. The method according to any one of claims 85-87, wherein the subject has a cancer that was determined to have one or more cells expressing the KRAS G12C mutant protein prior to administration of the compound, salt, or pharmaceutical composition.

89. The method according to any one of claims 85-88, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents.
